(19) United States
(12) Patent Application Publication
(10) Pub. No.: US 2023/0233569 A1
(43) Pub. Date: Jul. 27, 2023

(54) METHODS FOR DELAYING, PREVENTING, AND TREATING ACQUIRED RESISTANCE TO RAS INHIBITORS

(71) Applicant: Revolution Medicines, Inc., Redwood City, CA (US)

(72) Inventors: Mallika SINGH, Redwood City, CA (US); Jingjing JIANG, Redwood City, CA (US); Yu Chi YANG, Redwood City, CA (US); James W. EVANS, Redwood City, CA (US); Christopher J. SCHULZE, Redwood City, CA (US)

(21) Appl. No.: 18/011,454

(22) PCT Filed: Jun. 21, 2021
(86) PCT No.: PCT/US2021/037679

§ 371 Date: Dec. 19, 2022

Related U.S. Application Data
(60) Provisional application No. 63/041,071, filed on Jun. 18, 2020.
(60) Provisional application No. 63/062,973, filed on Aug. 7, 2020.
(60) Provisional application No. 63/117,417, filed on Nov. 23, 2020.
(60) Provisional application No. 63/134,128, filed on Jan. 5, 2021.
(60) Provisional application No. 63/192,976, filed on May 25, 2021.

(51) Int. Cl. A61K 31/519; A61K 31/416; A61K 47/54

(52) U.S. Cl. CPC A61K 31/519; A61K 31/416; A61K 47/545

(57) ABSTRACT

The present disclosure relates to compositions and methods for the treatment of diseases or disorders (e.g., cancer) with bi-steric inhibitors of mTOR in combination with RAS inhibitors. Specifically, in some embodiments this disclosure includes compositions and methods for inducing apoptosis of tumor cells and/or for delaying, preventing, or treating acquired resistance to RAS inhibitors using bi-steric mTOR inhibitors.

### CROSS-REFERENCE TO RELATED APPLICATIONS

This application claims the benefit of U.S. Provisional Application No. 63/041,071, filed Jun. 18, 2020 and U.S. Provisional Application No. 63/062,973, filed Aug. 7, 2020 and U.S. Provisional Application No. 63/117,417, filed Nov. 23, 2020, and U.S. Provisional Application No. 63/134,128, filed Jan. 5, 2021 and U.S. Provisional Application No. 63/192,976, filed May 25, 2021, the contents of each of which are incorporated herein by reference in their entireties.

### FIELD OF THE INVENTION

The present disclosure relates to compositions and methods for the treatment of diseases or disorders (e.g., cancer) with bi-steric mTOR inhibitors in combination with RAS inhibitors. Specifically, in some embodiments this disclosure includes compositions and methods for delaying, preventing, or treating acquired resistance to KRAS inhibitors using bi-steric mTOR inhibitors. In some embodiments, this disclosure includes compositions and methods for inducing apoptosis of a cell (e.g., a tumor cell) by contacting the cell with a RAS inhibitor (e.g., a KRAS(OFF) inhibitor such as a KRAS(OFF)^{G12C} inhibitor) in combination with a bi-steric mTOR inhibitor. In some particular embodiments, the present disclosure includes methods for inducing apoptosis of a cell (e.g., a tumor cell) by contacting the cell with a RAS inhibitor (e.g., a RAS(ON) inhibitor such as a KRAS(ON)^{G12C} inhibitor) in combination with a bi-steric mTOR inhibitor.

### BACKGROUND OF THE INVENTION

Cancer remains one of the most deadly threats to human health. In the U.S., cancer affects nearly 1.3 million new patients each year, and is the second leading cause of death after heart disease, accounting for approximately 1 in 4 deaths (US20170204187).

It has been well established in literature that RAS proteins (KRAS, HRAS and NRAS) play an essential role in various human cancers and are therefore appropriate targets for anticancer therapy. Dysregulation of RAS proteins by activating mutations, overexpression or upstream activation is common in human tumors, and activating mutations in RAS are found in approximately 30% of human cancer. Of the RAS proteins, KRAS is the most frequently mutated and is therefore an important target for cancer therapy. RAS oscillates between GDP-bound “off” (“RAS(OFF)”) and GTP-bound “on” (“RAS(ON)”) states, facilitated by interplay between a GEF protein (e.g., SOS1), which loads RAS with GTP, and a GAP protein (e.g., NF1), which hydrolyzes GTP, thereby inactivating RAS. Additionally, the SH2 domain-containing protein tyrosine phosphatase-2 (SHP2) associates with the receptor signaling apparatus and becomes active upon RTK activation, and then promotes RAS activation. Mutations in RAS proteins can lock the protein in the “on” state resulting in a constituitively active signaling pathway that leads to uncontrolled cell growth.

First-in-class covalent inhibitors of the “off” form of KRAS^{G12C} have demonstrated promising anti-tumor activity in cancer patients with KRAS^{G12C} mutations, albeit not in all. Further, therapeutic inhibition of the RAS pathway, although often initially efficacious, can ultimately prove ineffective as it may lead to over-activation of RAS pathway signaling via a number of mechanisms including, e.g., reactivation of the pathway via relief of the negative feedback machineries that naturally operate in these pathways. For example, in various cancers, MEK inhibition results in increased ErbB signaling due to its relief of MEK/ERK-mediated feedback inhibition of RTK activation. As a result, cells that were initially sensitive to such inhibitors may become resistant. Thus, a need exists for methods of effectively inhibiting RAS pathway signaling without inducing activation of resistance mechanisms, or by minimizing resistance mechanism effects.

### SUMMARY OF THE INVENTION

The present disclosure relates to compositions and methods for the treatment of diseases or disorders (e.g., cancer) with bi-steric inhibitors mTOR in combination with RAS inhibitors (e.g., KRAS(OFF) inhibitors such as KRAS(OFF)^{G12C}-selective inhibitors or KRAS(ON) inhibitors). Surprisingly, it has been found that such combinations can delay, prevent or treat acquired resistance to a RAS inhibitor. Specifically, in some embodiments this disclosure relates, in part, to compositions and methods for delaying, preventing, or treating acquired resistance to KRAS(OFF) inhibitors using bi-steric mTOR inhibitors. In some embodiments this disclosure relates to compositions and methods for delaying, preventing, or treating acquired resistance to KRAS(ON) inhibitors using bi-steric mTOR inhibitors. Moreover, it has been surprisingly found that apoptosis occurs in the presence of such combinations. Accordingly, in some embodiments, the disclosure relates to compositions and methods for inducing apoptosis of tumor cells using one or more bi-steric mTOR inhibitor in combination with one or more KRAS(OFF) inhibitor. In some embodiments, the disclosure relates to compositions and methods for inducing apoptosis of tumor cells using one or more bi-steric mTOR inhibitor in combination with one or more KRAS(ON) inhibitor.

In some embodiments, the present disclosure includes a method for delaying or preventing acquired resistance to a RAS inhibitor in a subject, comprising administering to the subject an effective amount of a bi-steric inhibitor of mTOR, wherein the subject has already received or will receive administration of the RAS inhibitor. In some embodiments, the RAS is selected from KRAS, NRAS, and HRAS. In some embodiments, the method further comprises administering to the subject an effective amount of the RAS inhibitor. In some embodiments, the RAS inhibitor targets a specific RAS mutation. In some embodiments, the RAS inhibitor targets a KRAS mutation. In some embodiments, the RAS inhibitor targets a G12C mutation. In some embodiments, the RAS inhibitor targets the KRAS^{G12C} mutation. In some embodiments, the RAS inhibitor binds the RAS in its “off” position. In some embodiments, the RAS inhibitor binds the RAS in its “on” position. In some embodiments, the RAS inhibitor is a KRAS(OFF) inhibitor. In some embodiments, the RAS inhibitor is a KRAS(ON) inhibitor. In some embodiments, the RAS inhibitor is selected from the inhibitors disclosed in any one of Appendices A-1, B-1, and C-1, or a RAS inhibitor of WO 2020132597 (wherein WO 2020132597 is incorporated by reference in its entirety), or a combination of two or more of such inhibitors. In some embodiments, the RAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation. In some embodiments, the KRAS inhibitor is selected from AMG 510, MRTX849, JDQ443 and MRTX1133. In some embodiments, the KRAS inhibitor is selected from AMG 510 and MRTX849. In some embodiments, the KRAS inhibitor is AMG 510. In some embodiments, the KRAS inhibitor is MRTX849. In some embodiments, the inhibitor of mTOR is RM-006, also known as RMC-6272, or RMC-5552. In some embodiments, the subject is administered the RAS inhibitor to treat or prevent a cancer. In some embodiments, the cancer is a G12C cancer. In some embodiments, the cancer comprises a KRAS^{G12C} mutation. In some embodiments, the cancer comprises co-occurring KRAS^{G12C} and STK11 mutations. In some embodiments, the cancer is a Non-Small Cell Lung Cancer (NSCLC). In some embodiments, the cancer is a colorectal cancer. In some embodiments, the cancer is selected from pancreatic cancer, colorectal cancer, non-small cell lung cancer, squamous cell lung carcinoma, thyroid gland adenocarcinoma, and a hematological cancer (e.g., blood (myeloid leukemia (acute and chronic), acute lymphoblastic leukemia, chronic lymphocytic leukemia); myeloproliferative diseases (myelofibrosis and myeloproliferative neoplasms); multiple myeloma; myelodysplastic syndromes). In some embodiments, the cancer comprises co-occurring KRAS^{G12C} and PIK3CA^{E545K} mutations. In some embodiments, the cancer is a colorectal cancer. In some embodiments, the method results in tumor regression. In some embodiments, the method results in tumor apoptosis.

In some embodiments, the present disclosure includes a method of treating acquired resistance to a RAS inhibitor in a subject, comprising administering to the subject an effective amount of a bi-steric inhibitor of mTOR. In some embodiments, the RAS is selected from KRAS, NRAS, and HRAS. In some embodiments, the method further comprises administering to the subject an effective amount of the RAS inhibitor. In some embodiments, the RAS inhibitor targets a specific RAS mutation. In some embodiments, the RAS inhibitor targets a KRAS mutation. In some embodiments, the RAS inhibitor targets a G12C mutation. In some embodiments, the RAS inhibitor targets the KRAS^{G12C} mutation. In some embodiments, the RAS inhibitor binds the RAS in its “off” position. In some embodiments, the RAS inhibitor binds the RAS in its “on” position. In some embodiments, the RAS inhibitor is a KRAS(OFF) inhibitor. In some embodiments, the RAS inhibitor is a KRAS(ON) inhibitor. In some embodiments, the RAS inhibitor is selected from the inhibitors disclosed in any one of Appendices A-1, B-1, and C-1, or a RAS inhibitor of WO 2020132597 (wherein WO 2020132597 is incorporated by reference in its entirety), or a combination of two or more of such inhibitors. In some embodiments, the RAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation. In some embodiments, the KRAS inhibitor is selected from AMG 510, MRTX849, JDQ443 and MRTX1133. In some embodiments, the KRAS inhibitor is selected from AMG 510 and MRTX849. In some embodiments, the KRAS inhibitor is AMG 510. In some embodiments, the KRAS inhibitor is MRTX849. In some embodiments, the inhibitor of mTOR is RM-006, also known as RMC-6272, or RMC-5552. In some embodiments, the subject is administered the RAS inhibitor to treat or prevent a cancer. In some embodiments, the cancer is a G12C cancer. In some embodiments, the cancer comprises a KRAS^{G12C} mutation. In some embodiments, the cancer comprises co-occurring KRAS^{G12C} and STK11 mutations. In some embodiments, the cancer is a Non-Small Cell Lung Cancer (NSCLC). In some embodiments, the cancer is a colorectal cancer. In some embodiments, the cancer is selected from pancreatic cancer, colorectal cancer, non-small cell lung cancer, squamous cell lung carcinoma, thyroid gland adenocarcinoma, and a hematological cancer (e.g., blood (myeloid leukemia (acute and chronic), acute lymphoblastic leukemia, chronic lymphocytic leukemia); myeloproliferative diseases (e.g., myelofibrosis and myeloproliferative neoplasms); multiple myeloma; myelodysplastic syndromes). In some embodiments, the cancer comprises co-occurring KRAS^{G12C} and PIK3CA^{E545K} mutations. In some embodiments, the cancer is a colorectal cancer. In some embodiments, the method results in tumor regression. In some embodiments, the method results in tumor apoptosis.

In some embodiments, the present disclosure includes a method of treating a subject having a cancer comprising administering to the subject a bi-steric inhibitor of mTOR in combination with a RAS inhibitor. In some embodiments, the RAS is selected from KRAS, NRAS, and HRAS. In some embodiments, the RAS inhibitor targets a specific RAS mutation. In some embodiments, the RAS inhibitor targets a KRAS mutation. In some embodiments, the RAS inhibitor targets a G12C mutation. In some embodiments, the RAS inhibitor targets the KRAS^{G12C} mutation. In some embodiments, the RAS inhibitor binds the RAS in its “off” position. In some embodiments, the RAS inhibitor is a KRAS(OFF) inhibitor. In some embodiments, the RAS inhibitor is a KRAS(ON) inhibitor. In some embodiments, the RAS inhibitor is selected from the inhibitors disclosed in any one of Appendices A-1, B-1, and C-1, or a RAS inhibitor of WO 2020132597 (wherein WO 2020132597 is incorporated by reference in its entirety), or a combination of two or more of such inhibitors. In some embodiments, the KRAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation. In some embodiments, the KRAS inhibitor is selected from AMG 510, MRTX849, JDQ443 and MRTX1133. In some embodiments, the KRAS inhibitor is selected from AMG 510 and MRTX849. In some embodiments, the KRAS inhibitor is AMG 510. In some embodiments, the KRAS inhibitor is MRTX849. In some embodiments, the bi-steric inhibitor of mTOR is RM-006, also known as RMC-6272, or RMC-5552. In some embodiments, the cancer is a G12C cancer. In some embodiments, the cancer comprises a KRAS^{G12C} mutation. In some embodiments, the cancer comprises co-occurring KRAS^{G12C} and STK11 mutations. In some embodiments, the cancer is a Non-Small Cell Lung Cancer (NSCLC). In some embodiments, the cancer is a colorectal cancer. In some embodiments, the cancer is selected from pancreatic cancer, colorectal cancer, non-small cell lung cancer, squamous cell lung carcinoma, thyroid gland adenocarcinoma, and a hematological cancer (e.g., blood (myeloid leukemia (acute and chronic), acute lymphoblastic leukemia, chronic lymphocytic leukemia); myeloproliferative diseases (e.g., myelofibrosis and myeloproliferative neoplasms); multiple myeloma; myelodysplastic syndromes). In some embodiments, the cancer comprises co-occurring KRAS^{G12C} and PIK3CA^{E545K} mutations. In some embodiments, the cancer is a colorectal cancer. In some embodiments, the method results in tumor regression. In some embodiments, the method results in tumor apoptosis.

In some embodiments, the present disclosure includes a method of inducing apoptosis of a tumor cell comprising contacting the tumor cell with a bi-steric inhibitor of mTOR in combination with a RAS inhibitor. In some embodiments, the RAS is selected from KRAS, NRAS, and HRAS. In some embodiments, the RAS inhibitor targets a specific RAS mutation. In some embodiments, the RAS inhibitor targets a KRAS mutation. In some embodiments, the RAS inhibitor targets a G12C mutation. In some embodiments, the RAS inhibitor targets the KRAS^{G12C} mutation. In some embodiments, the RAS inhibitor binds the RAS in its “off” position. In some embodiments, the RAS inhibitor is a KRAS(OFF) inhibitor. In some embodiments, the RAS inhibitor is a KRAS(ON) inhibitor. In some embodiments, the RAS inhibitor is selected from the inhibitors disclosed in any one of Appendices A-1, B-1, and C-1, or a RAS inhibitor of WO 2020132597 (wherein WO 2020132597 is incorporated by reference in its entirety), or a combination of two or more of such inhibitors. In some embodiments, the KRAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation. In some embodiments, the KRAS inhibitor is selected from AMG 510, MRTX849, JDQ443 and MRTX1133. In some embodiments, the KRAS inhibitor is selected from AMG 510 and MRTX849. In some embodiments, the KRAS inhibitor is AMG 510. In some embodiments, the KRAS inhibitor is MRTX849. In some embodiments, the inhibitor of mTOR is RM-006, also known as RMC-6272, or RMC-5552. In some embodiments, the tumor is caused by a cancer. In some embodiments, the cancer is a G12C cancer. In some embodiments, the cancer comprises a KRAS^{G12C} mutation. In some embodiments, the cancer comprises co-occurring KRAS^{G12C} and STK11 mutations. In some embodiments, the cancer is a Non-Small Cell Lung Cancer (NSCLC). In some embodiments, the cancer is a colorectal cancer. In some embodiments, the cancer is selected from pancreatic cancer, colorectal cancer, non-small cell lung cancer, squamous cell lung carcinoma, thyroid gland adenocarcinoma, and a hematological cancer (e.g., blood (myeloid leukemia (acute and chronic), acute lymphoblastic leukemia, chronic lymphocytic leukemia); myeloproliferative diseases (myelofibrosis and myeloproliferative neoplasms); multiple myeloma myelodysplastic syndromes). In some embodiments, the cancer comprises co-occurring KRAS^{G12C} and PIK3CA^{E545K} mutations. In some embodiments, the cancer is a colorectal cancer. In some embodiments, the method results in tumor regression. In some embodiments, the method results in tumor apoptosis. In some embodiments, the method results in an improved lifespan for the subject as compared to the lifespan of a similar subject that has not received a treatment with the RAS inhibitor and the bi-steric mTOR inhibitor.

### BRIEF DESCRIPTION OF THE DRAWINGS

FIG. 1 shows the combinatorial anti-proliferative activity of RM-006 (also known as RMC-6272) and the KRAS^{G12C}(OFF) inhibitor AMG 510 in the NSCLC cells lines NCI-H2122 and NCI-H2030, which each have with RAS and mTOR signaling co-activation. FIG. 1A shows the anti-proliferative activity that resulted from varying concentrations of AMG 510 in presence of constant RM-006 (also known as RMC-6272) (3 nM in H2122, left panel, and 10 nM in H2030, right panel). FIG. 1B shows the anti-proliferative activity that resulted from varying concentrations of RM-006 (also known as RMC-6272) in presence of constant AMG 510 (90 nM in H2122, left panel, or 10 nM in H2030, right panel).

FIG. 2 shows that RM-006 (also known as RMC-6272) enhances the in vivo anti-tumor activity of a KRAS^{G12C}(OFF) inhibitor, and the combination of these compounds delays tumor regrowth. FIG. 2A shows a tumor volume plot demonstrating the combinatorial effects of RM-006 (also known as RMC-6272) with AMG 510 on in vivo tumor growth in the human non-small cell lung cancer NCI-H358 KRAS^{G12C} xenograft model. FIG. 2B shows a waterfall plot presenting the end of study responses of each mouse tested in FIG. 2A. FIG. 2C shows a tumor volume plot demonstrating the combinatorial effects of RM-006 (also known as RMC-6272) with AMG 510 on in vivo tumor growth delay following treatment cessation. FIG. 2D shows Kaplan-Meier analysis demonstrating a significant delay in tumor regrowth back to 500 mm3 after treatment cessation caused by the combination of AMG 510 with RM-006 (also known as RMC-6272) as compared to single-agent AMG 510, and as assessed by Log-rank (Mantel-Cox) test with p=0.0395.

FIG. 3 shows that the combination of RM-006 (also known as RMC-6272) and KRAS^{G12C}(OFF) inhibition drives tumor regression in the NCI-H2122 NSCLC model, which has co-activation of RAS and mTOR signaling. FIG. 3A shows a tumor volume plot demonstrating the in vivo tumor growth inhibition induced by RM-006 (also known as RMC-6272) and AMG 510 alone or in combination in the NCI-H2122 NSCLC CDX model. ***=p<0.001, assessed by an ordinary one-way ANOVA of tumor volumes along with multiple comparisons via a post-hoc Tukey's test in GraphPad Prism software. FIG. 3B shows a waterfall plot demonstrating individual tumor responses at the end of the study.

FIG. 4 shows results of a single-dose PKPD study using NCI-H2122 NSCLC CDX. Pathway modulation was assessed by quantitative image analyses of IHC staining of tumor sections for pS6RP (S235) (FIG. 4A); p4EBP1 (FIG. 4B); pERK (FIG. 4C); and by qPCR assay for human DUSP6 (FIG. 4D). FIG. 4E shows representative IHC staining images for pS6RP and FIG. 4F shows representative IHC staining images for p4EBP1.

FIG. 5 shows synergistic in vivo induction of apoptosis in human non-small cell lung cancer NCI-H2122 KRAS^{G12C}; STK11del tumors induced by a single dose of the RM-006 (also known as RMC-6272) in combination with AMG 510. FIG. 5A shows quantification of IHC staining for cleaved caspase 3 (CC3). FIG. 5B shows representative CC3 staining 24 (top row images) and 48 hrs. (bottom row images) post-treatment with the indicated amounts of RM-006 (also known as RMC-6272) and AMG 510 alone and in combination.

FIG. 6 shows that the combination of RM-006 (also known as RMC-6272) and a KRAS^{G12C}(OFF) inhibitor significantly delays on-treatment resistance in a NSCLC model with RAS and mTOR signaling co-activation. FIG. 6A shows a mean tumor volume plot demonstrating significant delay in on-treatment resistance induced by co-treatment with RM-006 (also known as RMC-6272) and AMG 510 as compared to single agent treatment. FIG. 6B shows Kaplan-Meier analysis of tumors reaching baseline volume while on treatment, and the results demonstrate the combination significantly prolonged the time for tumors to develop resistance, as assessed by Log-rank (Mantel-Cox) test.

FIG. 7 shows tumor volume plots of four mice demonstrating attenuation of AMG 510 xenograft tumor resistance in the NCI-H2030 model by treatment with RM-006 (also known as RMC-6272). N=4.

FIG. 8 shows combinatorial activity of RM-006 (also known as RMC-6272) and a KRAS^{G12C}(OFF) inhibitor in the ST3235 (KRAS^{G12C} PIK3CA^{E545K}) CRC PDX model.

FIG. 9 shows combinatorial activity of RM-006 (also known as RMC-6272) and a RAS(ON) inhibitor of the disclosure, Compound A, on tumor cell growth in vivo were evaluated in the human lung cancer ST1989 KRAS^{G12C} patient-derived xenograft model using female athymic nude mice (6-12 weeks old).

FIG. 10 shows combinatorial effects of RMC-6272 (also known as RMC-006) with Compound B in a NSCLC CDX Model.

FIG. 11 shows combinatorial effects of RMC-5552 with Compound B in a NSCLC CDX Model.

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used in this disclosure to refer to one or more than one (i.e., to at least one) of the grammatical object of the article. By way of example, “an element” means one element or more than one element.

The term “or” is used to mean “and/or” unless explicitly indicated to refer to alternatives only or the alternative are mutually exclusive, although the disclosure supports a definition that refers to only alternatives and “and/or”. The term “and/or” is used in this disclosure to mean either “and” or “or” unless indicated otherwise.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “e.g.” is used herein to mean “for example,” and will be understood to imply the inclusion of a stated step or element or group of steps or elements but not the exclusion of any other step or element or group of steps or elements.

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The terms “bi-steric mTOR inhibitor” and “bi-steric inhibitor of mTOR” are used interchangeably in this disclosure to refer to two pharmacophores in a single compound. One pharmacophore binds to the well-known FRB (FKBP12-rapamycin binding) site on mTORC1 and the other binds to the mTOR kinase active site. As a result of these two binding interactions, such compounds exhibit two biologically useful features: (1) selectivity for mTORC1 over mTORC2, which is characteristic of the natural compound rapamycin, and (2) deep inhibition of mTORC1, which is characteristic of known active site inhibitors. These properties enable selective inhibition of phosphorylation of mTORC1 substrates, including 4EBP1. In some embodiments, a bi-steric mTOR inhibitor has a molecular weight of between 1600 and 2100 Da, inclusive, and exhibits selective (>10-fold) inhibition of mTORC1 over mTORC2.

The term “carrier”, as used in this disclosure, encompasses excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “combination therapy” refers to a method of treatment comprising administering to a subject at least two therapeutic agents, optionally as one or more pharmaceutical compositions. For example, a combination therapy may comprise administration of a single pharmaceutical composition comprising at least two therapeutic agents and one or more pharmaceutically acceptable carrier, excipient, diluent, and/or surfactant. A combination therapy may comprise administration of two or more pharmaceutical compositions, each composition comprising one or more therapeutic agent and one or more pharmaceutically acceptable carrier, excipient, diluent, and/or surfactant. In various embodiments, at least one of the therapeutic agents is a bi-steric mTOR inhibitor (e.g., any one or more such bi-steric mTOR inhibitor disclosed herein or known in the art). In various embodiments, at least one of the therapeutic agents is a KRAS(OFF) inhibitor (e.g., any one or more KRAS(OFF) inhibitor disclosed herein or known in the art). In some particular embodiments, at least one of the therapeutic agents is a KRAS^{G12C} inhibitor (e.g., any one or more of the KRAS^{G12C} inhibitors disclosed herein or known in the art). In some particular embodiments, at least one of the therapeutic agents is AMG 510, MRTX849, JDQ443 or MRTX1133. In some embodiments, the at least one of the therapeutic agents is selected from AMG 510 and MRTX849. In some embodiments, the therapeutic agent is AMG 510. In some embodiments, the therapeutic agent is MRTX849. In various embodiments, at least one of the therapeutic agents is a bi-steric mTOR inhibitor and one of the therapeutic agents is a KRAS^{G12C} inhibitor. The two agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due an additive or synergistic effect of combining the two or more therapeutics.

The term “disorder” is used in this disclosure to mean, and is used interchangeably with, the terms disease, condition, or illness, unless otherwise indicated.

An “effective amount” when used in connection with a compound is an amount effective for treating or preventing a disease or disorder in a subject as described herein.

The term “inhibitor” means a compound that prevents a biomolecule, (e.g., a protein, nucleic acid) from completing or initiating a reaction. An inhibitor can inhibit a reaction by competitive, uncompetitive, or non-competitive means. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor can be nucleic acid molecules including, but not limited to, siRNA that reduce the amount of functional protein in a cell. Accordingly, compounds said to be “capable of inhibiting” a particular protein, e.g., mTOR or RAS, comprise any such inhibitor.

As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS). In some embodiments, a RAS(OFF) inhibitor has a molecular weight of under 700 Da. The term “KRAS(OFF) inhibitor” refers to any inhibitor that binds to KRAS in its GDP-bound “OFF” position. Reference to the term KRAS(OFF) inhibitor includes, for example, AMG 510, MRTX849, JDQ443 and MRTX1133. In some embodiments, the KRAS(OFF) inhibitor is selected from AMG 510 and MRTX849. In some embodiments, the KRAS(OFF) inhibitor is AMG 510. In some embodiments, the KRAS(OFF) inhibitor is MRTX849. In some embodiments, the KRAS(OFF) inhibitor is selected from BPI-421286, JNJ-74699157 (ARS-3248), LY3537982, MRTX1257, ARS853, ARS1620, or GDC-6036. In some embodiments, reference to the term KRAS(OFF) inhibitor includes any such KRAS(OFF) inhibitor disclosed in any one of the following patent applications: WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223 each of which are incorporated herein by reference in its entirety. Reference to “AMG 510” and “MRTX849” herein means the following compounds:

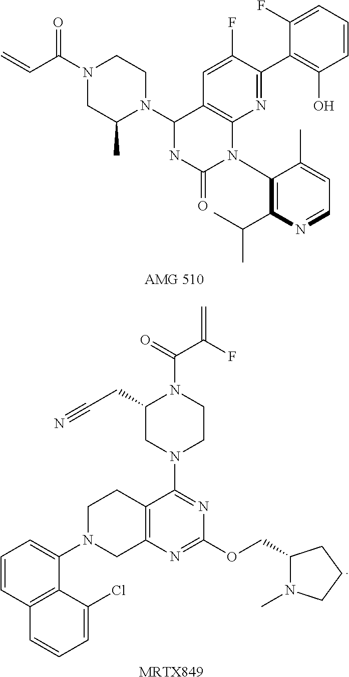

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). In some embodiments, a RAS(ON) inhibitor has a molecular weight of between 800 and 1100 Da, inclusive. The term “KRAS(ON) inhibitor” refers to any inhibitor that binds to KRAS in its GDP-bound “ON” position. Reference to the term KRAS(ON) inhibitor includes, without limitation, any one or more KRAS(ON) inhibitor selected from the KRAS(ON) inhibitors disclosed in Appendix A-1, Appendix B-1, and Appendix C-1, or a RAS inhibitor of WO 2020132597 (wherein WO 2020132597 is incorporated by reference in its entirety), or a combination of any such KRAS(ON) inhibitors.

As used herein, “Compound A” and “Compound B” are each distinct KRAS^{G12C}(ON) inhibitors disclosed in Appendix B-1, and encompass pharmaceutically acceptable salts thereof unless otherwise explicitly indicated otherwise.

The term “monotherapy” refers to a method of treatment comprising administering to a subject a single therapeutic agent, optionally as a pharmaceutical composition. For example, a monotherapy may comprise administration of a pharmaceutical composition comprising a therapeutic agent and one or more pharmaceutically acceptable carrier, excipient, diluent, and/or surfactant. The therapeutic agent may be administered in an effective amount. The therapeutic agent may be administered in a therapeutically effective amount.

The term “mutation” as used herein indicates any modification of a nucleic acid and/or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The term “preventing acquired resistance,” as used herein, means avoiding the occurrence of acquired or adaptive resistance. Thus, e.g., the use of a bi-steric mTOR inhibitor described herein in preventing acquired/adaptive resistance to a KRAS^{G12C} inhibitor means that the bi-steric mTOR inhibitor is administered prior to any detectable existence of resistance to the KRAS^{G12C} inhibitor and the result of such administration of the bi-steric mTOR inhibitor is that no resistance to the KRAS^{G12C} inhibitor occurs.

The term “providing to a/the subject” a therapeutic agent, e.g., a bi-steric mTOR inhibitor, includes administering such an agent.

The terms “RAS inhibitor” and “inhibitor of [a] RAS” are used interchangeably to refer to any inhibitor that targets a RAS protein. In various embodiments, these terms include RAS(OFF) and RAS(ON) inhibitors such as, e.g., the KRAS(OFF) and KRAS(ON) inhibitors disclosed herein. The term “RAS(OFF) inhibitor” refers to any inhibitor that binds to a RAS protein in its GDP-bound “OFF” position, as further defined herein. The term “RAS(ON) inhibitor” refers to any inhibitor that binds to a RAS protein in its GDP-bound “ON” position, as further defined herein. In some embodiments, a RAS inhibitor has a molecular weight of under 700 Da. In some embodiments, the RAS inhibitor is selected from the group consisting of AMG 510, MRTX1257, JNJ-74699157 (ARS-3248), LY3537982, ARS-853, ARS-1620, GDC-6036, BPI-421286, JDQ443, JAB-21000, JAB-22000, and JAB-23000. A RAS inhibitor may be a RAS vaccine, or another therapeutic modality designed to directly or indirectly decrease the oncogenic activity of RAS.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The term RM-006 (also known as RMC-6272) refers to a bi-steric mTOR inhibitor (also termed an mTORC1-selective inhibitor), which has the following structure:

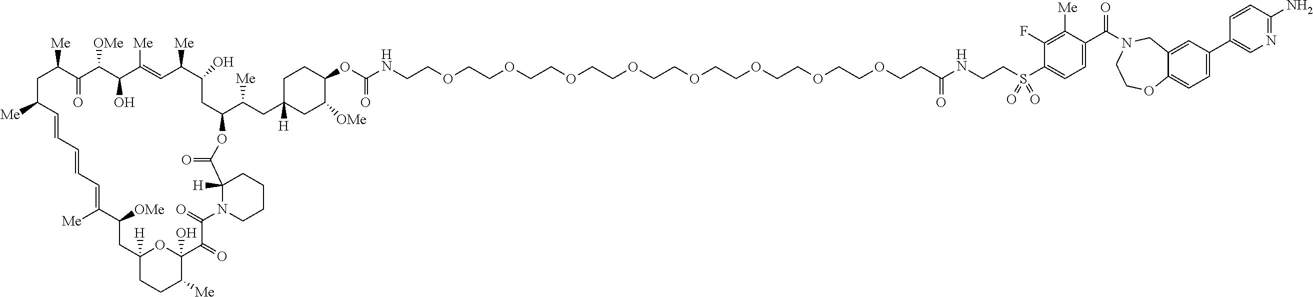 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

The term RMC-5552 refers to a bi-steric mTOR inhibitor (also termed an mTORC1-selective inhibitor), found in Appendix D-1 and in WO 2019212990, wherein WO 2019212990 is incorporated herein by reference in its entirety, which has the following structure:

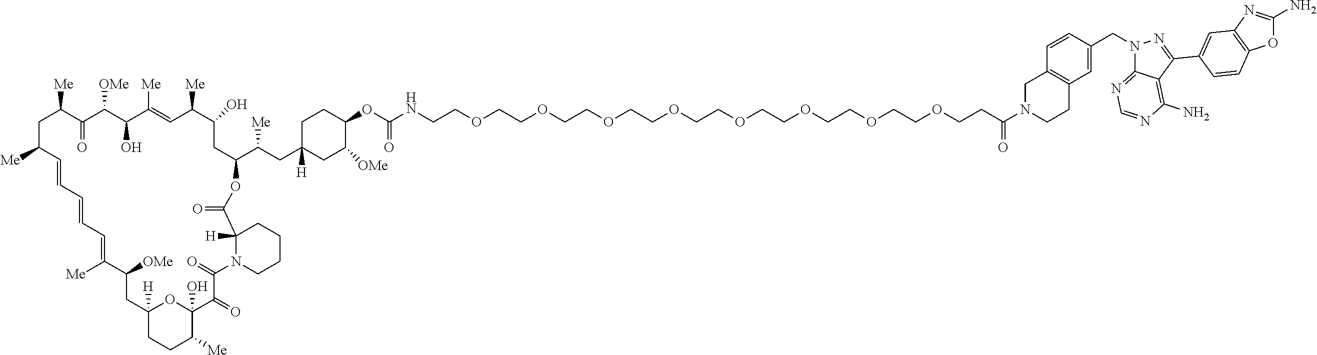 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Reference to a “subtype” of a cell (e.g., a KRAS^{G12C} subtype, a KRAS^{G12S} subtype, a KRAS^{G12D} subtype, a KRAS^{G12V} subtype) means that the cell contains a gene mutation encoding a change in the protein of the type indicated. For example, a cell classified as a “KRAS^{G12C} subtype” contains at least one KRAS allele that encodes an amino acid substitution of cysteine for glycine at position 12 (^{G12C}) and, similarly, other cells of a particular subtype (e.g. KRAS^{G12D}, KRAS^{G12S} and KRAS^{G12V} subtypes) contain at least one allele with the indicated mutation (e.g., a KRAS^{G12D} mutation, a KRAS^{G12S} mutation or a KRAS^{G12V} mutation, respectively). Unless otherwise noted, all amino acid position substitutions referenced herein (such as, e.g., “^{G12C}” in KRAS^{G12C}) correspond to substitutions in the human version of the referenced protein, i.e., KRAS^{G12C} refers to a G→C substitution in position 12 of human KRAS.

A “therapeutic agent” is any substance, e.g., a compound or composition, capable of treating a disease or disorder. In some embodiments, therapeutic agents that are useful in connection with the present disclosure include without limitation mTOR inhibitors, RAS inhibitors such as, e.g., KRAS inhibitors (e.g., KRAS^{G12C} inhibitors), and cancer chemotherapeutics. Many such inhibitors are known in the art and are disclosed herein.

The terms “therapeutically effective amount”, “therapeutic dose”, “prophylactically effective amount”, or “diagnostically effective amount” is the amount of the drug, e.g., a bi-steric mTOR inhibitor, needed to elicit the desired biological response following administration.

The term “treatment” or “treating” with regard to a subject, refers to improving at least one symptom, pathology or marker of the subject's disease or disorder, either directly or by enhancing the effect of another treatment. Treating includes curing, improving, or at least partially ameliorating the disorder, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

### Overview

The present disclosure relates to, inter alia, compositions, methods, and kits for treating or preventing a disease or disorder (e.g., cancer) with a RAS inhibitor (e.g., a KRAS^{G12C} inhibitor) in combination with a bi-steric mTOR inhibitor. In some particular embodiments, the present disclosure includes methods for delaying, preventing, or treating acquired resistance to a RAS inhibitor (e.g., a KRAS^{G12C} inhibitor) by administering the RAS inhibitor (e.g., a KRAS^{G12C} inhibitor) in combination with a bi-steric mTOR inhibitor. In some particular embodiments, the present disclosure includes methods for inducing apoptosis of a cell (e.g., a tumor cell) by contacting the cell with a RAS inhibitor (e.g., a KRAS(OFF) inhibitor such as a KRAS^{G12C} inhibitor) in combination with a bi-steric mTOR inhibitor. In some particular embodiments, the present disclosure includes methods for inducing apoptosis of a cell (e.g., a tumor cell) by contacting the cell with a RAS inhibitor (e.g., a RAS(ON) inhibitor such as a KRAS(ON)^{G12C} inhibitor) in combination with a bi-steric mTOR inhibitor.

The mammalian target of rapamycin (mTOR) is a serine-threonine kinase related to the lipid kinases of the phosphoinositide 3-kinase (PI3K) family. mTOR exists in two complexes, mTORC1 and mTORC2, which are differentially regulated, have distinct substrate specificities, and are differentially sensitive to rapamycin. mTORC1 integrates signals from growth factor receptors with cellular nutritional status and controls the level of cap-dependent mRNA translation by modulating the activity of key translational components such as the cap-binding protein and oncogene eIF4E. Hyperactivation of the PI3K/mTOR pathway occurs frequently in human cancer, via mutation or deletion of different components.

Various inhibitors of mTOR exist and have differential specificity for the two mTOR complexes. However, despite clear biological rationale, PI3K/mTOR pathway inhibitors have been largely unsuccessful in “all-comers” clinical trials, attributed to the lack of biomarker-guided patient stratification. The present inventors have developed a class of selective mTORC1 inhibitors, termed ‘bi-steric’, which comprise a rapamycin-like core moiety covalently linked to an mTOR active-site inhibitor. Bi-steric mTORC1 inhibitors exhibit potent and selective (>10-fold) inhibition of mTORC1 over mTORC2, durably suppress S6K and 4EBP1 phosphorylation, and induce growth suppression and apoptosis in multiple cancer cell lines. These inhibitors provide the mTORC1 selectivity of rapalogs and potently inhibit translation initiation by the 4EBP1-eIF4E axis while sparing mTORC2. In various embodiments, any one or more of these bi-steric mTOR inhibitors may utilized in any of methods disclosed herein.

Accordingly, in some embodiments, the present disclosure relates to the unexpected discovery that acquired resistance to KRAS inhibitors, and in particular KRAS^{G12C} inhibitors, can be delayed and even arrested or reversed by coadministration of a bi-steric mTOR inhibitor (e.g., such as RM-006, also known as RMC-6272, or RMC-5552). Moreover, in some embodiments, the present disclosure relates to the unexpected discovery that the combination of KRAS inhibitors, and in particular KRAS^{G12C} inhibitors with a bi-steric mTOR inhibitor (e.g., such as RM-006, also known as RMC-6272, or RMC-5552) results in synergistic apoptosis of tumor cells. Thus, in some embodiments, the present disclosure includes compositions, methods, and kits for the treatment of a disease or condition (e.g., a cancer or tumor) with a RAS inhibitor in combination with a bi-steric mTOR inhibitor. In particular embodiments, the RAS inhibitor targets KRAS, NRAS, or HRAS. In particular embodiments the RAS inhibitor is a RAS mutant specific inhibitor. In certain embodiments, RAS mutant is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof.
   Mutations at these positions may result in RAS-driven tumors. In some particular embodiments, the RAS inhibitor is a KRAS(OFF) inhibitor known in the art or disclosed herein. The KRAS(OFF) inhibitor may be any one or more of the KRAS(OFF) inhibitors disclosed in any one of WO 2020118066, WO 2020113071, WO 2020106647, WO 2020106640, WO 2020102730, WO 2020101736, WO 2020097537, WO 2020086739, WO 2020018282, WO 2020050890, WO 2020047192, WO 2020035031, WO 2020033413, WO 2020028706, WO 2019241157, WO 2019234405, WO 2019232419, WO 2019227040, WO 2019217933, WO 2019217691, WO 2019217307, WO 2019215203, WO 2019213526, WO 2019213516, WO 2019204442, WO 2019204449, WO 2019204505, WO 2019155399, WO 2019150305, WO 2019137985, WO 2019110751, WO 2019099524, WO 2019055540, WO 2019051291, WO 2018237084, WO 2018218070, WO 2018217651, WO 2018218071, WO 2018218069, WO 2018212774, WO 2018206539, WO 2018195439, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2018011351, WO 2018005678, WO 2017201161, WO 20171937370, WO 2017172979, WO 2017112777, WO 2017106520, WO 2017096045, WO 2017100546, WO 2017087528, WO 2017079864, WO 2017058807, WO 2017058805, WO 2017058728, WO 2017058902, WO 2017058792, WO 2017058768, WO 2017058915, WO 2017015562, WO 2016179558, WO 2016176338, WO 2016168540, WO 2016164675, WO 2016100546, WO 2016049568, WO 2016049524, WO 2015054572, WO 2014152588, WO 2014143659 and WO 2013155223, each of which is incorporated herein by reference in its entirety. In one such embodiment, the disclosure includes compositions, methods, and kits for the treatment of a disease or condition (e.g., a cancer or tumor) with a bi-steric mTOR inhibitor and KRAS(OFF) inhibitor selected from AMG 510, MRTX849, JDQ443 and MRTX1133. In some embodiments, the KRAS(OFF) inhibitor is selected from AMG 510 and MRTX849. In some embodiments, the KRAS(OFF) inhibitor is AMG 510. In some embodiments, the KRAS(OFF) inhibitor is MRTX849. In some particular embodiments, the RAS inhibitor is a KRAS(ON) inhibitor known in the art or disclosed herein. The KRAS(ON) inhibitor may be any one or more of the KRAS(ON) inhibitors disclosed in any one of Appendices A-1, B-1, and C-1, or a RAS inhibitor of WO 2020132597 (wherein WO 2020132597 is incorporated by reference in its entirety). The bi-steric mTOR inhibitor utilized in any such methods may in some embodiments be any bi-steric mTOR inhibitor known in the art or disclosed herein. In some embodiments, the bi-steric mTOR inhibitor is selected from any one of more of the bi-steric mTOR inhibitors disclosed in WO 2016/040806, WO 2018/204416, WO 2019/212990, or WO 2019/212991, each of which is incorporated herein by reference in its entirety. In some embodiments, the bi-steric mTOR inhibitor may be any one or more bi-steric mTOR inhibitors disclosed in Appendix D-1.

In some embodiments, the mTOR inhibitor is RM-006 (also known as RMC-6272).

In some embodiments, the mTOR inhibitor is RMC-5552. In some embodiments, the bi-steric mTOR inhibitor is

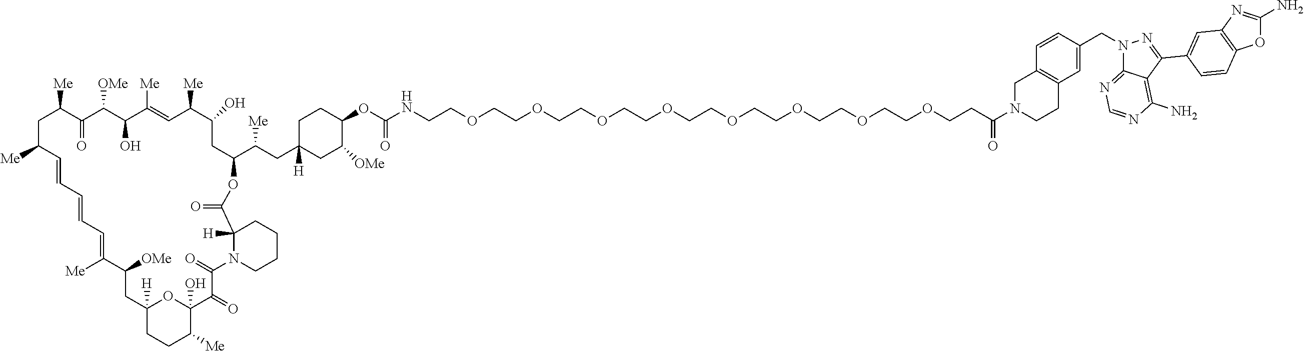 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer thereof. In some embodiments, the bi-steric mTOR inhibitor is

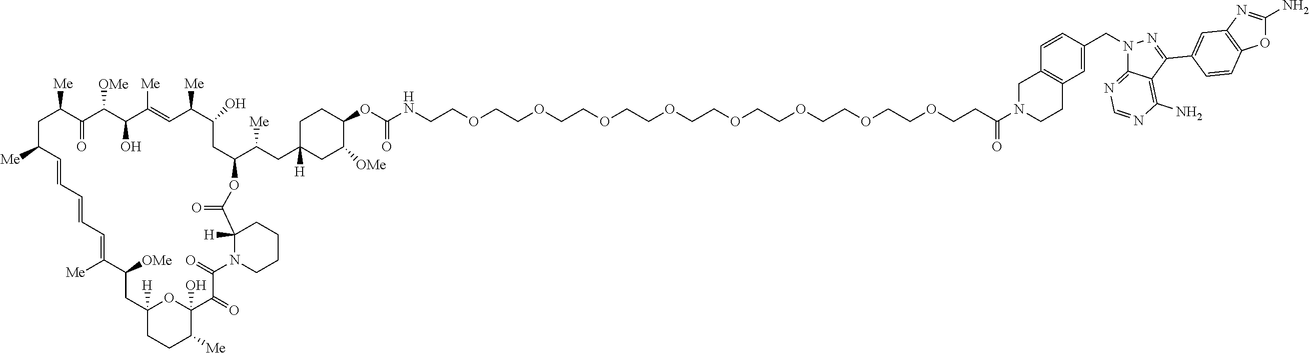 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a tautomer thereof. In some embodiments, the bi-steric mTOR inhibitor is

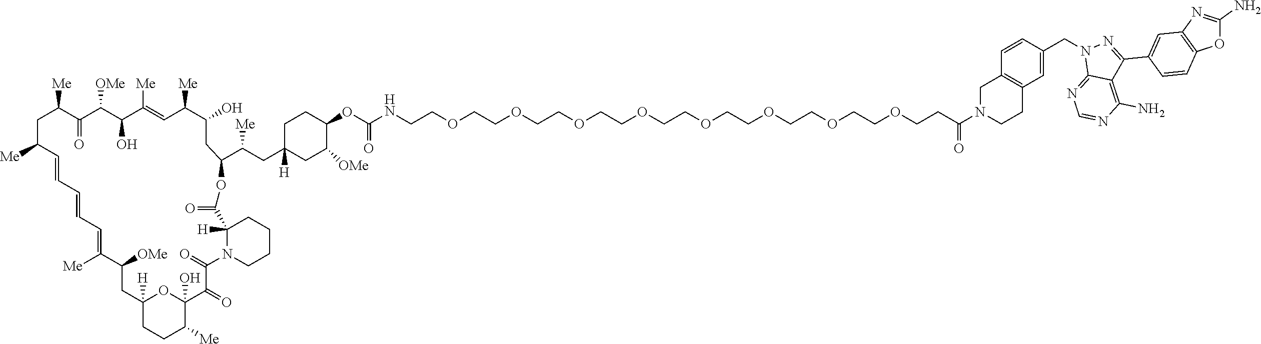 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or an oxepane isomer thereof, such as described in WO 2019212990, incorporated herein by reference in its entirety. In some embodiments, the bi-steric mTOR inhibitor is

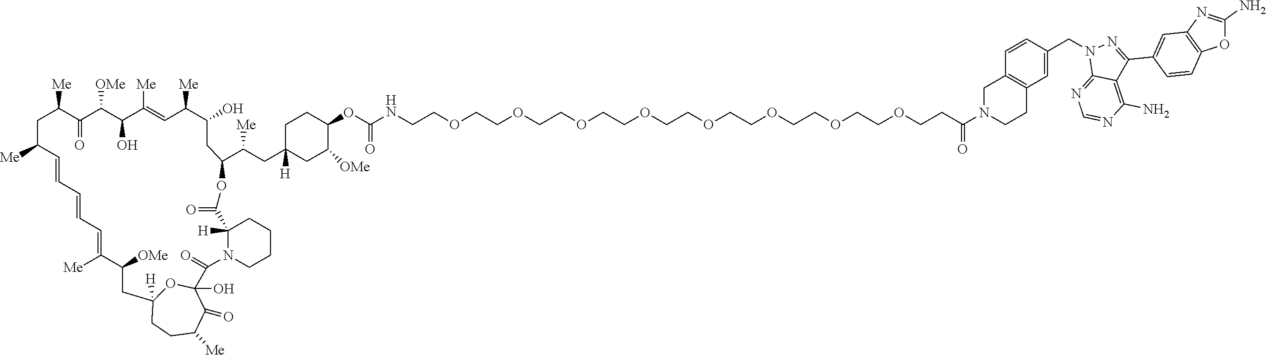 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer thereof. In some embodiments, the bi-steric mTOR inhibitor is

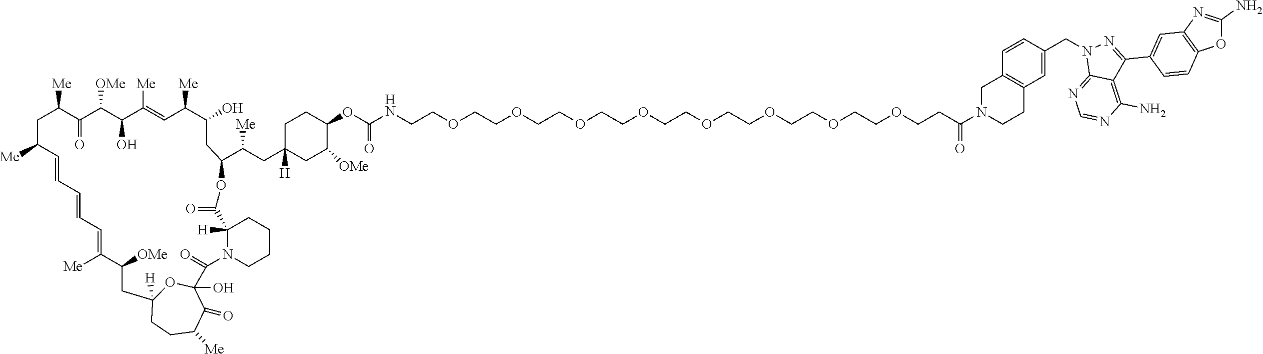 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a tautomer thereof. In some embodiments, the bi-steric mTOR inhibitor is

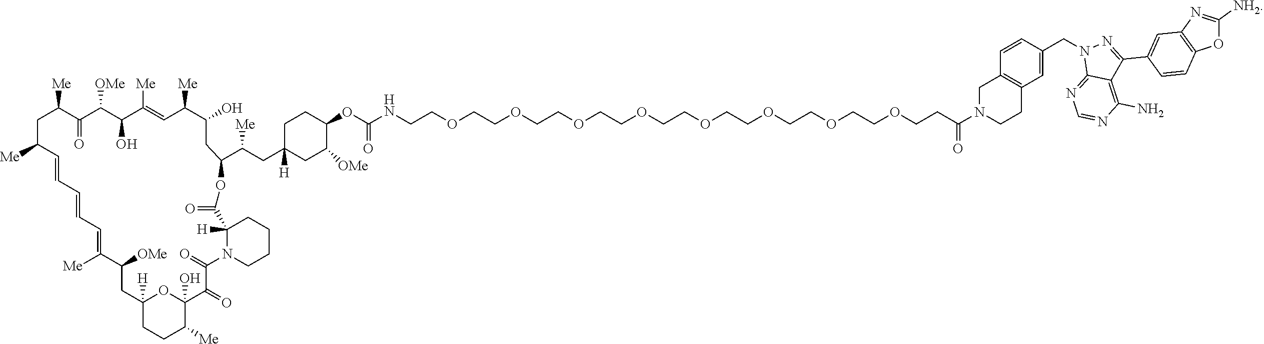 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In some embodiments, the bi-steric mTOR inhibitor is

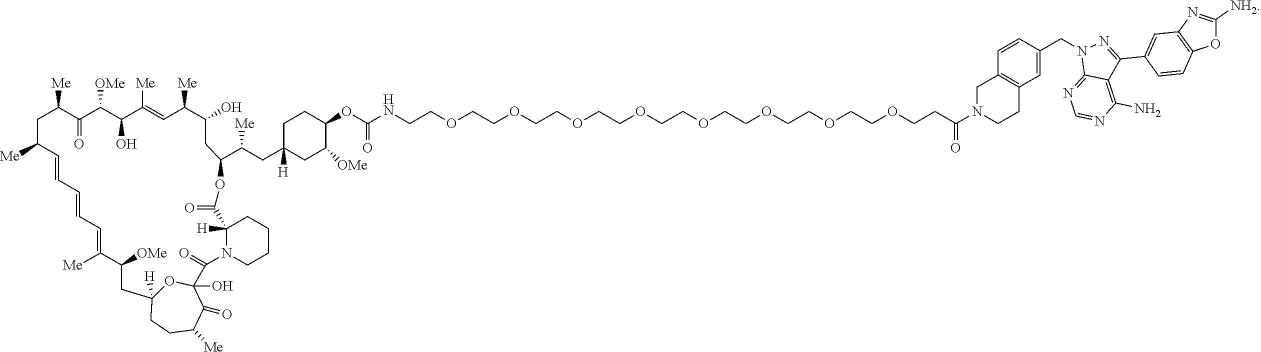 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In some embodiments, a composition is provided comprising

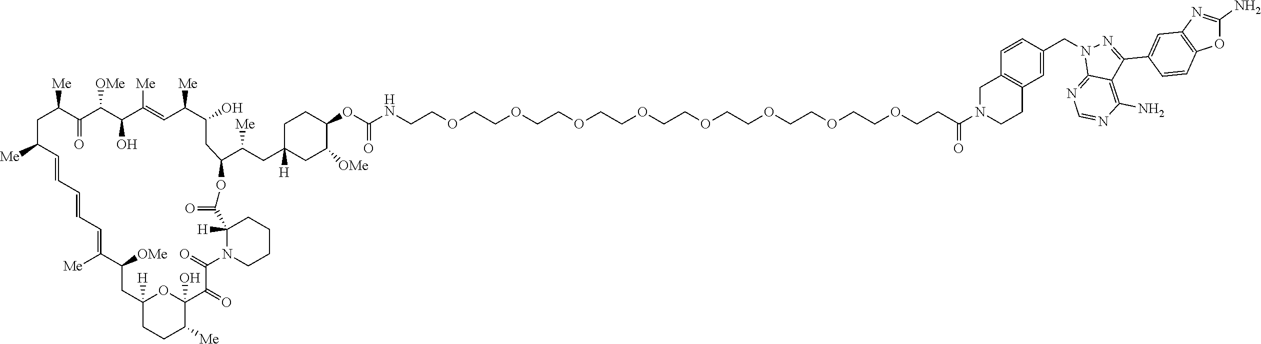 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer or tautomer thereof
and

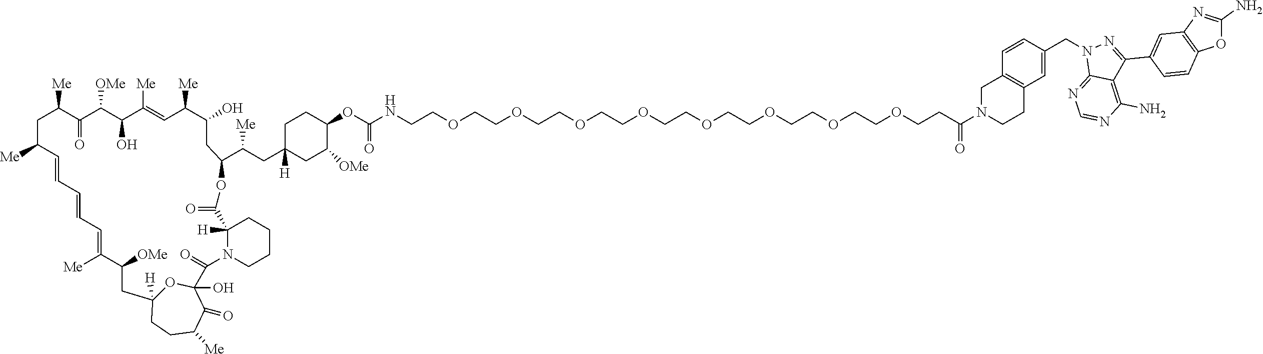 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer or tautomer thereof. The composition may further comprise a pharmaceutically acceptable excipient. In some embodiments, a composition is provided comprising

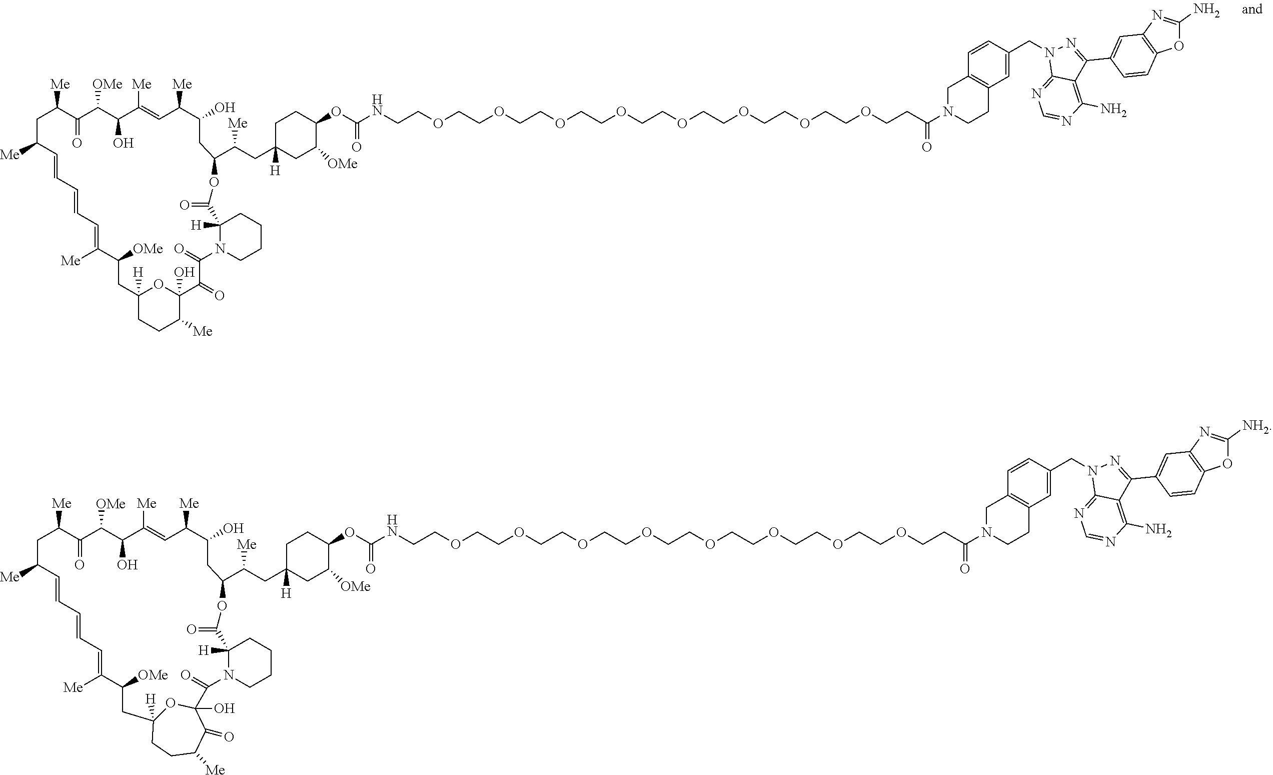

The composition may further comprise a pharmaceutically acceptable excipient.

Any disease or condition treatable with a RAS inhibitor may be treated according to the present disclosure. The treatment may be in a subject in need thereof. The compounds (e.g., bi-steric mTOR inhibitor and/or RAS inhibitor, such as a KRAS^{G12C} inhibitor) may be administered to treat the disease or condition (e.g., cancer or a tumor) in an effective amount. In particular embodiments, the disease or condition that is treated according to the methods disclosed herein is a cancer. The cancer may form a tumor. For example, the present disclosure provides a method of treating cancer in a subject in need thereof, the method comprising administering to the subject a therapeutically effective amount of a compound of the present invention (e.g., a bi-steric mTOR inhibitor disclosed herein or known in the art and/or RAS inhibitor, such as a KRAS^{G12C} inhibitor disclosed herein or known in the art), or a pharmaceutically acceptable salt thereof, or a pharmaceutical composition comprising such a compound or salt.

In some embodiments, the cancer comprises a RAS mutation. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. Also provided is a method of treating a Ras protein-related disorder in a subject in need thereof, the method comprising administering to the subject a therapeutically effective amount of a compound of the present invention (e.g., a bi-steric mTOR inhibitor disclosed herein or known in the art and/or RAS inhibitor, such as a KRAS^{G12C} inhibitor disclosed herein or known in the art), or a pharmaceutically acceptable salt thereof, or a pharmaceutical composition comprising such a compound or salt.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:

Cardiac, for example: sarcoma (angiosarcoma, fibrosarcoma, rhabdomyosarcoma, liposarcoma), myxoma, rhabdomyoma, fibroma, lipoma, and teratoma;

Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver, for example: hepatoma (hepatocellular carcinoma), cholangiocarcinoma, hepatoblastoma, angiosarcoma, hepatocellular adenoma, hemangioma;

Biliary tract, for example: gall bladder carcinoma, ampullary carcinoma, cholangiocarcinoma;

Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;

Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);

Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic, for example: blood (myeloid leukemia (acute and chronic), acute lymphoblastic leukemia, chronic lymphocytic leukemia); myeloproliferative diseases (e.g., myelofibrosis and myeloproliferative neoplasms); multiple myeloma; myelodysplastic syndromes, Hodgkin's disease, non-Hodgkin's lymphoma (malignant lymphoma);

Skin, for example: malignant melanoma, basal cell carcinoma, squamous cell carcinoma, Kaposi's sarcoma, moles dysplastic nevi, lipoma, angioma, dermatofibroma, keloids, psoriasis; and

Adrenal glands, for example: neuroblastoma.

In some embodiments, the disease or condition that is treated according to the methods disclosed herein is a RAS G12C cancer. As used herein, the term “G12C cancer” means a cancer that comprises one or more G12C mutation. Such mutations can occur in HRAS, NRAS, and KRAS.

In some embodiments, the disease or condition that is treated according to the methods disclosed herein is pancreatic cancer, colorectal cancer, non-small cell lung cancer, squamous cell lung carcinoma, thyroid gland adenocarcinoma, or a hematological cancer.

In some embodiments, the present disclosure includes a method of delaying or preventing acquired resistance to a RAS inhibitor in a subject, comprising administering to the subject a bi-steric inhibitor of mTOR, wherein the subject has already received or will receive administration of the RAS inhibitor. In particular embodiments, the RAS inhibitor targets KRAS, NRAS, or HRAS. In particular embodiments the RAS inhibitor is a RAS mutant specific inhibitor. In certain embodiments, RAS mutant is selected from
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;

(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof.
   In some particular embodiments, the RAS inhibitor is a KRAS(OFF) inhibitor known in the art or disclosed herein. In some embodiments, the disclosure includes compositions, methods, and kits for the delaying or preventing acquired resistance to a KRAS(OFF) inhibitor selected from AMG 510, MRTX849, JDQ443 and MRTX1133, the method comprising administering to the subject a bi-steric mTOR inhibitor. In some embodiments, the KRAS(OFF) inhibitor is selected from AMG 510 and MRTX849. In some embodiments, the KRAS(OFF) inhibitor is AMG 510. In some embodiments, the KRAS(OFF) inhibitor is MRTX849. In some particular embodiments, the RAS inhibitor is a KRAS(ON) inhibitor known in the art or disclosed herein. The KRAS(ON) inhibitor may be any one or more of the KRAS(ON) inhibitors disclosed in any one of Appendices A-1, B-1, and C-1, or a RAS inhibitor of WO 2020132597 (wherein WO 2020132597 is incorporated by reference in its entirety). The bi-steric mTOR inhibitor utilized in any such methods may in some embodiments be any bi-steric mTOR inhibitor known in the art or disclosed herein. In some embodiments, the bi-steric mTOR inhibitor is selected from any one of more of the bi-steric mTOR inhibitors disclosed in WO 2016/040806, WO 2018/204416, WO 2019/212990, or WO 2019/212991, each of which is incorporated herein by reference in its entirety. In some embodiments, the bi-steric mTOR inhibitor is RM-006 (also known as RMC-6272). In some embodiments, the bi-steric mTOR inhibitor is RMC-5552. The subject may have a cancer, e.g., any one of more of the cancers disclosed herein. The cancer may be a G12C cancer.

In some embodiments, the present disclosure includes a method of treating acquired resistance to a RAS inhibitor in a subject, comprising administering to the subject a bi-steric inhibitor of mTOR, wherein the subject has already received administration of the RAS inhibitor and developed resistance to the RAS inhibitor. In particular embodiments, the RAS inhibitor targets KRAS, NRAS, or HRAS. In particular embodiments the RAS inhibitor is a RAS mutant specific inhibitor. In certain embodiments, RAS mutant is selected from
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof.
   In some embodiments, the disclosure includes compositions, methods, and kits for treating acquired resistance to a KRAS(OFF) inhibitor selected from AMG 510, MRTX849, JDQ443, and MRTX1133, the method comprising administering to the subject a bi-steric mTOR inhibitor, wherein the subject has already received administration of the RAS inhibitor and developed resistance to the RAS inhibitor. In some embodiments, the KRAS(OFF) inhibitor is selected from AMG 510 and MRTX849. In some embodiments, the KRAS(OFF) inhibitor is AMG 510. In some embodiments, the KRAS(OFF) inhibitor is MRTX849. In some particular embodiments, the RAS inhibitor is a KRAS(ON) inhibitor known in the art or disclosed herein. The KRAS(ON) inhibitor may be any one or more of the KRAS(ON) inhibitors disclosed in any one of Appendices A-1, B-1, and C-1, or a RAS inhibitor of WO 2020132597 (wherein WO 2020132597 is incorporated by reference in its entirety). The bi-steric mTOR inhibitor utilized in any such methods may in some embodiments be any bi-steric mTOR inhibitor known in the art or disclosed herein. In some embodiments, the bi-steric mTOR inhibitor is selected from any one of more of the bi-steric mTOR inhibitors disclosed in WO 2016/040806, WO 2018/204416, WO 2019/212990, or WO 2019/212991, each of which is incorporated herein by reference in its entirety. In some embodiments, bi-steric mTOR inhibitor is RM-006 (also known as RMC-6272). In some embodiments, the bi-steric inhibitor of mTOR is RMC-5552. The subject may have a cancer, e.g., any one of more of the cancers disclosed herein. The cancer may be a G12C cancer.

In various embodiments, the methods described herein for treating such diseases or conditions, and for treating, delaying or preventing acquired resistance to a RAS inhibitor in a subject, comprising administering to the subject a bi-steric inhibitor of mTOR, involve administering to a subject an effective amount of a bi-steric mTOR inhibitor, a RAS inhibitor (e.g., a KRAS^{G12C} inhibitor), or a composition (e.g., a pharmaceutical composition) comprising such a bi-steric mTOR inhibitor, a RAS inhibitor (e.g., a KRAS^{G12C} inhibitor), or a combination thereof. In some such embodiments, the RAS inhibitor is a KRAS(OFF) inhibitor known in the art or disclosed herein. In some such embodiments, the RAS inhibitor is a KRAS(ON) inhibitor known in the art or disclosed herein.

Any compound or substance capable of inhibiting RAS may be utilized in application with the present disclosure to inhibit RAS. Non-limiting examples of such RAS inhibitors are known in the art and are disclosed herein. For example, the compositions and methods described herein may utilize one or more RAS inhibitor selected from, but not limited to, any KRAS(OFF) inhibitor disclosed herein or known in the art. The KRAS(OFF) inhibitor may be any one or more KRAS(OFF) inhibitor disclosed in any one of WO 2020118066, WO 2020113071, WO 2020106647, WO 2020106640, WO 2020102730, WO 2020101736, WO 2020097537, WO 2020086739, WO 2020018282, WO 2020050890, WO 2020047192, WO 2020035031, WO 2020033413, WO 2020028706, WO 2019241157, WO 2019234405, WO 2019232419, WO 2019227040, WO 2019217933, WO 2019217691, WO 2019217307, WO 2019215203, WO 2019213526, WO 2019213516, WO 2019204442, WO 2019204449, WO 2019204505, WO 2019155399, WO 2019150305, WO 2019137985, WO 2019110751, WO 2019099524, WO 2019055540, WO 2019051291, WO 2018237084, WO 2018218070, WO 2018217651, WO 2018218071, WO 2018218069, WO 2018212774, WO 2018206539, WO 2018195439, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2018011351, WO 2018005678, WO 2017201161, WO 20171937370, WO 2017172979, WO 2017112777, WO 2017106520, WO 2017096045, WO 2017100546, WO 2017087528, WO 2017079864, WO 2017058807, WO 2017058805, WO 2017058728, WO 2017058902, WO 2017058792, WO 2017058768, WO 2017058915, WO 2017015562, WO 2016179558, WO 2016176338, WO 2016168540, WO 2016164675, WO 2016100546, WO 2016049568, WO 2016049524, WO 2015054572, WO 2014152588, WO 2014143659 and WO 2013155223, each of which is incorporated herein by reference in its entirety. In various embodiments, the compositions and methods described herein utilize the KRAS(OFF) inhibitor AMG 510. In various embodiments, the compositions and methods described herein utilize the KRAS(OFF) inhibitor MRTX849. In various embodiments, the compositions and methods described herein utilize the KRAS(OFF) inhibitor JDQ443. In various embodiments, the compositions and methods described herein utilize the KRAS(OFF) inhibitor MRTX1133. In some embodiments, the compositions and methods described herein utilize a RAS inhibitor that is a KRAS(ON) inhibitor known in the art or disclosed herein. The KRAS(ON) inhibitor may be any one or more of the KRAS(ON) inhibitors disclosed in any one of Appendices A-1, B-1, and C-1, or a RAS inhibitor of WO 2020132597 (wherein WO 2020132597 is incorporated by reference in its entirety). The compositions and methods described herein may utilize one or more bi-steric mTOR inhibitor selected from, but not limited to any bi-steric mTOR inhibitor disclosed in WO 2016/040806, WO 2018/204416, WO 2019/212990, and WO 2019/212991, each of which is incorporated herein by reference in its entirety.

The bi-steric mTOR inhibitor may be administered alone as a monotherapy or in combination with one or more other therapeutic agent (e.g., a RAS inhibitor such as a KRAS(OFF) inhibitor a KRAS(ON) inhibitor and/or an anti-cancer therapeutic agent) as a combination therapy. The bi-steric mTOR inhibitor and/or the RAS inhibitor (e.g., KRAS(OFF) inhibitor or KRAS(ON) inhibitor) may be administered as a pharmaceutical composition. The bi-steric mTOR inhibitor may be administered before, after, and/or concurrently with the one or more other therapeutic agent (e.g., a RAS inhibitor and/or an anti-cancer therapeutic agent). For example, the bi-steric mTOR inhibitor may be administered before, after, and/or concurrently with a KRAS^{G12C} inhibitor. The bi-steric mTOR inhibitor may be administered before, after, and/or concurrently with AMG 510. The bi-steric mTOR inhibitor may be administered before, after, and/or concurrently with MRTX849. The bi-steric mTOR inhibitor may be administered before, after, and/or concurrently with JDQ443. The bi-steric mTOR inhibitor may be administered before, after, and/or concurrently with MRTX1133. The bi-steric mTOR inhibitor may be administered before, after, and/or concurrently with a RAS(ON) inhibitor (e.g., a KRAS(ON) inhibitor). The bi-steric mTOR inhibitor may be administered before, after, and/or concurrently with a RAS(ON) inhibitor disclosed in any one of Appendices A-1, B-1, and C-1, or a RAS inhibitor of WO 2020132597 (wherein WO 2020132597 is incorporated by reference in its entirety). If the bi-steric mTOR inhibitor is administered concurrently with the one or more other therapeutic agent, such administration may be simultaneous (e.g., in a single composition) or may be via two or more separate compositions, optionally via the same or different modes of administration (e.g., local, systemic, oral, intravenous, etc.).

In certain embodiments, the bi-steric mTOR inhibitor is administered to the subject as a monotherapy for the treatment of a cancer associated with a mutation in a RAS gene. The RAS gene mutation may be a KRAS, NRAS, or HRAS mutation. Oncogenic RAS mutations, such as KRAS mutations, shift the RAS equilibrium to the GTP-bound “on” state, driving signaling to RAS effectors and oncogene addiction. As used herein, “oncogene addiction” refers to the phenomenon whereby a tumor cell exhibits apparent dependence on a single oncogenic pathway or protein for sustained proliferation and/or survival, despite its myriad of genetic alterations. In certain embodiments, the bi-steric mTOR inhibitor is administered to the subject as a monotherapy for the treatment of a cancer associated with a KRAS^{G12C} mutation. In certain embodiments, the bi-steric mTOR inhibitor is administered to the subject as a monotherapy for the treatment of a cancer associated with a KRAS^{G12A}; a KRAS^{G12D}, a KRAS^{G12S}, or a KRAS^{G12V} mutation, or any other RAS mutation described herein.

In certain embodiments, the bi-steric mTOR inhibitor is administered to the subject in combination with one or more other therapeutic agent (e.g., a RAS inhibitor) as a combination therapy for the treatment of a cancer associated with a mutation in a RAS gene. The mutation may be in KRAS, NRAS or HRAS. The mutation may comprise one or more of a KRAS mutation selected from a KRAS^{G12A} mutation; a KRAS^{G12C} mutation; a KRAS^{G12D} mutation; a KRAS^{G12S} mutation; and a KRAS^{G12V} mutation. The combination therapy may comprise administration of a bi-steric mTOR inhibitor and any RAS inhibitor known in the art or disclosed herein. For example, the bi-steric mTOR inhibitor may be administered to the subject in combination with a KRAS(OFF) inhibitor known in the art or disclosed herein. The bi-steric mTOR inhibitor may be administered to the subject in combination with AMG 510. The bi-steric mTOR inhibitor may be administered to the subject in combination with MRTX849. The bi-steric mTOR inhibitor may be administered to the subject in combination with JDQ443. The bi-steric mTOR inhibitor may be administered to the subject in combination with MRTX1133. The bi-steric mTOR inhibitor may be administered to the subject in combination with a RAS(ON) inhibitor (e.g., a KRAS(ON) inhibitor). The bi-steric mTOR inhibitor may be administered to the subject in combination with a RAS(ON) inhibitor disclosed any one or more of Appendices A-1, B-1, and C-1, or a RAS inhibitor of WO 2020132597 (wherein WO 2020132597 is incorporated by reference in its entirety). The mTOR inhibitor and optionally the RAS inhibitor may also be administered in combination with one or more other therapeutic agent. In some embodiments, the other therapeutic agent used in combination is selected from JNJ-74699157; LY3499446; MRTX1257; ARS 1620; and a combination thereof. MRTX1257 and ARS 1620 have the following structures, respectively:

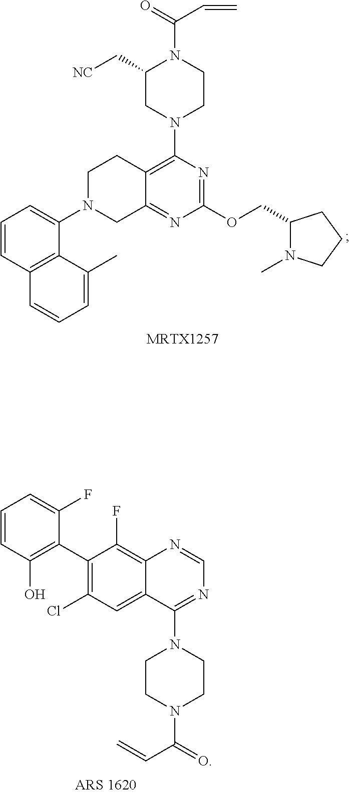

### Combination Therapy

The methods of the invention may include a compound of the invention used alone or in combination with one or more additional therapies (e.g., non-drug treatments or therapeutic agents). In various embodiments, “compound of the invention” refers to any of the compounds described herein. For example, in particular embodiments, the term “compound of the invention” includes any one of more of the RAS inhibitors (e.g., KRAS inhibitors) disclosed herein and any one or more of the bi-steric mTOR inhibitors disclosed herein. In various embodiments, it is contemplated that reference to any one of the compounds disclosed herein (e.g., any one of more of the RAS inhibitors (e.g., KRAS inhibitors) disclosed herein and any one or more of the bi-steric mTOR inhibitors disclosed herein, as well as any other therapeutic agents described herein) also may include a salt of such a compound, such as a pharmaceutically acceptable salt. The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment). For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. In some embodiments, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GALS, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab).

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BMW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CS1 (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torc1/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TN0155, RMC-4550, RMC-4630, JAB-3068, JAB-3312, RLY-1971, ERAS-601, or BBP-398), an SOS1 inhibitor (e.g., BI-1701963, BI-3406), a Raf inhibitor, a MEK inhibitor, an ERK inhibitor, a PI3K inhibitor, a PTEN inhibitor, or an AKT inhibitor. In some embodiments, the anti-cancer agent is JAB-3312.

In some embodiments, a therapeutic agent that may be combined with a compound of the present invention is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, a therapeutic agent may be a pan-RTK inhibitor, such as afatinib.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl] phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and applications: WO 2021110796; WO 2021088945; WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US 20210085677, U.S. Ser. No. 10/988,466, U.S. Ser. No. 10/858,359, U.S. Ser. No. 10/934,302 and U.S. Ser. No. 10/954,243, each of which is incorporated herein by reference in its entirety.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TN0155. In some embodiments, the SHP2 inhibitor is RMC-4550. In some embodiments, the SHP2 inhibitor is RMC-4630, whose structure is shown below:

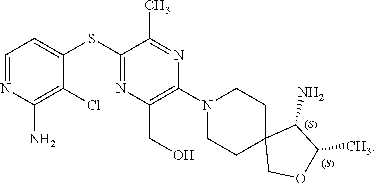

In some embodiments, the SHP2 inhibitor is JAB-3068.

In some embodiments, the additional therapeutic agent is selected from the group consisting of a HER2 inhibitor, a SHP2 inhibitor, a CDK4/6 inhibitor, an SOS1 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019).

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAG1, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATGS (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-la, interferon beta-1b, interferon gamma, natural interferon gamma-la, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levami sole+fluorouracil, liarozole, lobaplatin, lonidamine, lovastatin, masoprocol, melarsoprol, metoclopramide, mifepri stone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzig); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

As one of ordinary skill in the art will appreciate, in various embodiments, all of the therapeutic agents disclosed herein, i.e., the specific bi-steric mTOR inhibitors, RAS inhibitors (e.g., KRAS(OFF) inhibitors, KRAS^{G12C} specific inhibitors, KRAS(ON) inhibitors), TKI inhibitors, MEK inhibitors, ALK inhibitors, SHP2 inhibitors, EGFR inhibitors, etc., may be used in any one or more of the embodiments disclosed herein that call for such an inhibitor, generally. Thus, for example, an embodiment comprising treatment with, e.g., a “bi-steric mTOR inhibitor,” generally, or a “RAS inhibitor,” generally, may comprise treatment with any one or more bi-steric mTOR inhibitor or RAS inhibitor, respectively, that is disclosed herein (unless context requires otherwise).

Administration of the disclosed compositions and compounds (e.g., bi-steric mTOR inhibitors, RAS inhibitors (e.g., KRAS(OFF) inhibitors and/or KRAS(ON) inhibitors) and/or other therapeutic agents) can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts. Pharmaceutical compositions suitable for the delivery of a bi-steric mTOR inhibitor and a RAS inhibitor (e.g., a KRAS(OFF) inhibitor or a KRAS(ON) inhibitor) (alone or, e.g., in combination with another therapeutic agent according to the present disclosure) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein in its entirety.

Illustrative pharmaceutical compositions are tablets and gelatin capsules comprising a bi-steric mTOR inhibitor, a RAS inhibitor (e.g., a KRAS(OFF) inhibitor and/or a KRAS(ON) inhibitor) alone or in combination with one another and/or in combination with another therapeutic agent according to the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, a bi-steric mTOR inhibitor, a RAS inhibitor (e.g., a KRAS(OFF) inhibitor and/or a KRAS(ON) inhibitor) alone or in combination with one another and/or in combination with another therapeutic agent according to the disclosure) is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the SHP2 inhibitor (alone or in combination with another therapeutic agent according to the disclosure).

A bi-steric mTOR inhibitor and/or a RAS inhibitor (e.g., a KRAS(OFF) inhibitor and/or a KRAS(ON) inhibitor) alone or in combination with one another and/or in combination with another therapeutic agent can be also formulated as a suppository, alone or in combination with another therapeutic agent according to the disclosure, which can be prepared from fatty emulsions or suspensions; using polyalkylene glycols such as propylene glycol, as the carrier.

A bi-steric mTOR inhibitor and/or a RAS inhibitor (e.g., a KRAS(OFF) inhibitor and/or a KRAS(ON) inhibitor) alone or in combination with one another and/or in combination with another therapeutic agent can also be administered in the form of liposome delivery systems, such as small unilamellar vesicles, large unilamellar vesicles and multilamellar vesicles, either alone or in combination with another therapeutic agent according to the disclosure. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

A bi-steric mTOR inhibitor and/or a RAS inhibitor (e.g., a KRAS(OFF) inhibitor and/or a KRAS(ON) inhibitor) alone or in combination with one another and/or in combination with another therapeutic agent inhibitors can also be delivered by the use of monoclonal antibodies as individual carriers to which the disclosed compounds are coupled. Bi-steric mTOR inhibitor and/or the RAS inhibitor (e.g., a KRAS(OFF) inhibitor) alone or in combination with one another and/or in combination with another therapeutic agent inhibitors can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolyly sine substituted with palmitoyl residues. Furthermore, a bi-steric mTOR inhibitor and/or a RAS inhibitor (e.g., a KRAS(OFF) inhibitor and/or a KRAS(ON) inhibitor) can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In some embodiments, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a bi-steric mTOR inhibitor and/or the RAS inhibitor (e.g., a KRAS(OFF) inhibitor) alone or in combination with one another and/or in combination with another therapeutic agent according to the present disclosure and a pharmaceutically acceptable carrier. The pharmaceutically acceptable carrier can further include an excipient, diluent, or surfactant.

Thus, the present disclosure provides compositions (e.g., pharmaceutical compositions) comprising one or more bi-steric mTOR inhibitors for use in a method disclosed herein. Such compositions may comprise a bi-steric mTOR inhibitors inhibitor and, e.g., one or more carrier, excipient, diluent, and/or surfactant. The present disclosure provides compositions (e.g., pharmaceutical compositions) comprising one or more RAS inhibitors (e.g., a KRAS(OFF) inhibitor) for use in a method disclosed herein. Such compositions may comprise a RAS inhibitor (e.g., a KRAS(OFF) inhibitor) and, e.g., one or more carrier, excipient, diluent, and/or surfactant. The present disclosure provides compositions (e.g., pharmaceutical compositions) comprising one or more bi-steric mTOR inhibitors and one or more RAS inhibitors (e.g., a KRAS(OFF) inhibitor) for use in a method disclosed herein. Such compositions may comprise one or more bi-steric mTOR inhibitors inhibitor and one or more RAS inhibitor (e.g., a KRAS(OFF) inhibitor) e.g., one or more carrier, excipient, diluent, and/or surfactant. Such compositions may also comprise one or more additional therapeutic agent for use in a method disclosed herein, such as, e.g., a SHP2 inhibitor, a TKI, a MAPK pathway inhibitor, an EGFR inhibitor, an ALK inhibitor, and or a MEK inhibitor and, e.g., one or more carrier, excipient, diluent, and/or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed Compound By weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of a bi-steric mTOR inhibitor, when used for the indicated effects, range from about 0.1 mg to about 1000 mg as needed to treat the condition. Compositions for in vivo or in vitro use can contain about 0.1, 0.2, 0.3, 0.4, 0.5, 5, 20, 50, 75, 100, 150, 250, 500, 750, or 1000 mg of the disclosed compound, or, in a range of from one amount to another amount in the list of doses. In some embodiments, compositions for in vivo or in vitro use contain from 0.5 mg to 500 mg (e.g., from about 1 mg to about 400 mg). In some embodiments, the compositions are in the form of an intravenous solution.

Effective dosage amounts of an ALK inhibitor, when used for the indicated effects, range from about 0.5 mg to about 5000 mg as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In some embodiments, the compositions are in the form of a tablet that can be scored.

Effective dosage amounts of an EGFR inhibitor, when used for the indicated effects, range from about 0.5 mg to about 5000 mg as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In some embodiments, the compositions are in the form of a tablet that can be scored.

Effective dosage amounts of an MEK inhibitor, when used for the indicated effects, range from about 0.05 mg to about 5000 mg as needed to treat the condition. Compositions for in vivo or in vitro use can contain about 0.05, 0.1, 0.2, 0.3, 0.4, 0.5, 0.6, 0.7, 0.8, 0.9, 1, 2, 5, 20, 50, 75, 100, 150, 250, 500, 750, 1000, 1250, 2500, 3500, or 5000 mg of the disclosed compound, or, in a range of from one amount to another amount in the list of doses. In some embodiments, the compositions are in the form of a tablet that can be scored.

The present invention also provides kits for treating a disease or disorder with a bi-steric mTOR inhibitor and optionally a RAS inhibitor (e.g., a KRAS(OFF) inhibitor and/or a KRAS(ON) inhibitor), one or more carrier, excipient, diluent, and/or surfactant, and a means for determining whether a sample from a subject (e.g., a tumor sample) is likely to be sensitive to such a bi-steric mTOR and/or RAS inhibitor treatment. In some embodiments, the means for determining comprises a means for determining whether the sample comprises a RAS mutation, e.g., a NRAS, KRAS, or HRAS mutation. Such mutations may comprise a G12C mutation. Such mutations may be selected from a KRAS^{G12C} mutation, a KRAS^{G12D} mutation, a KRAS^{G12S} mutation, and/or a KRAS^{G12V} mutation. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro.

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds and fluorescence is detected. In some embodiments, the KRAS, HRAS or NRAS G12C mutation is identified using a direct sequencing method of specific regions (e.g., exon 2 and/or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

Methods for determining whether a tumor or cancer comprises a G12C or other KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

### EXEMPLARY EMBODIMENTS

Some embodiments of this disclosure are in the Embodiments, as follows:

Embodiment I-1. A method for delaying or preventing acquired resistance to a RAS inhibitor in a subject in need thereof, comprising administering to the subject an effective amount of a bi-steric inhibitor of mTOR, wherein the subject has already received or will receive administration of the RAS inhibitor, wherein the effective amount is an amount effective to delay or prevent acquired resistance to the RAS inhibitor in a subject in need thereof.
Embodiment I-2. A method of treating acquired resistance to a RAS inhibitor in a subject in need thereof, comprising administering to the subject an effective amount of a bi-steric inhibitor of mTOR, wherein the effective amount is an amount effective to treat acquired resistance to the RAS inhibitor in a subject in need thereof.
Embodiment I-3. The method of Embodiment I-1 or I-2, wherein the RAS is selected from KRAS, NRAS, and HRAS.
Embodiment I-4. The method of any one of Embodiments I-1 to I-3, further comprising administering to the subject an effective amount of the RAS inhibitor.
Embodiment I-5. The method of any one of Embodiments I-1 to I-4 wherein the RAS inhibitor targets a specific RAS mutation.
Embodiment I-6. The method of any one of Embodiments I-1 to I-5, wherein the RAS inhibitor targets a KRAS mutation.
Embodiment I-7. The method of any one of Embodiments I-1 to I-6, wherein the RAS inhibitor targets a G12C mutation.
Embodiment I-8. The method of any one of Embodiments I-1 to I-7, wherein the RAS inhibitor targets the KRAS^{G12C} mutation.
Embodiment I-9. The method of any one of Embodiments I-1 to I-8, wherein the RAS inhibitor binds the RAS in its “off” position.
Embodiment I-10. The method of any one of Embodiments I-6 to I-9, wherein the RAS inhibitor is a KRAS(OFF) inhibitor.
Embodiment I-11. The method of any one of Embodiments I-1 to I-6 or Embodiments I-9 to I-10, wherein the RAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation.
Embodiment I-12. The method of any one of Embodiments I-1 to I-11, wherein the KRAS inhibitor is selected from AMG 510, MRTX849, JDQ443 and MRTX1133, or a pharmaceutically acceptable salt thereof.
Embodiment I-13. The method of any one of the preceding Embodiments, wherein the bi-steric inhibitor of mTOR is RM-006, also known as RMC-6272, or RMC-5552, or a pharmaceutically acceptable salt thereof.
Embodiment I-14. The method of any one of Embodiments I-1 to I-12, wherein the bi-steric inhibitor of mTOR is a compound having the formula

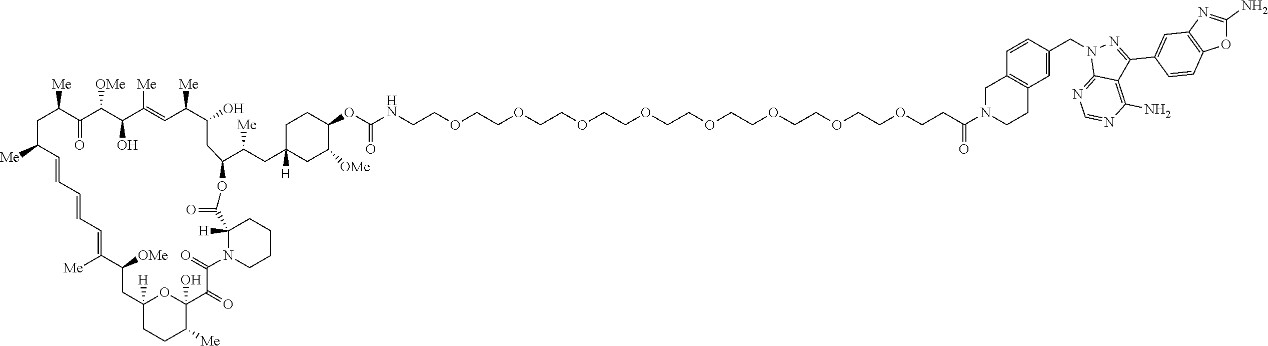 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer thereof.
Embodiment I-15. The method of any one of Embodiments I-1 to I-12, wherein the bi-steric inhibitor of mTOR is a compound having the formula

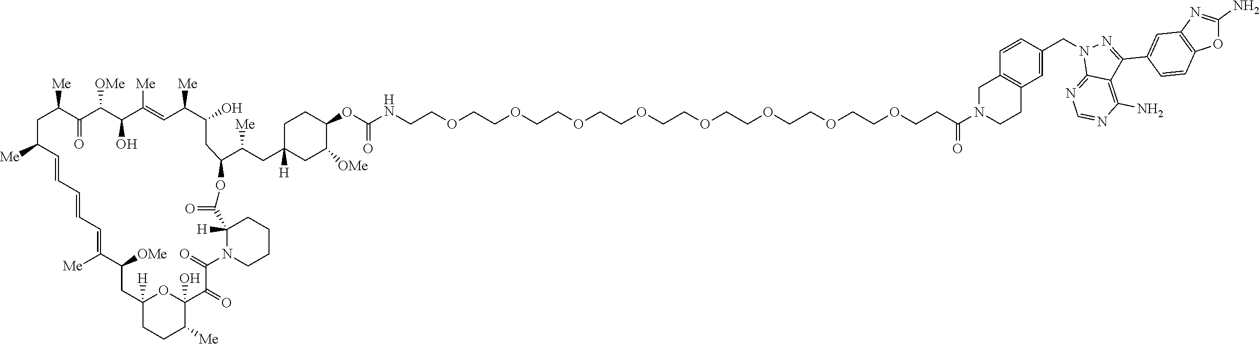 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a tautomer thereof.
Embodiment I-16. The method of any one of Embodiments I-1 to I-12, wherein the bi-steric inhibitor of mTOR is a compound having the formula

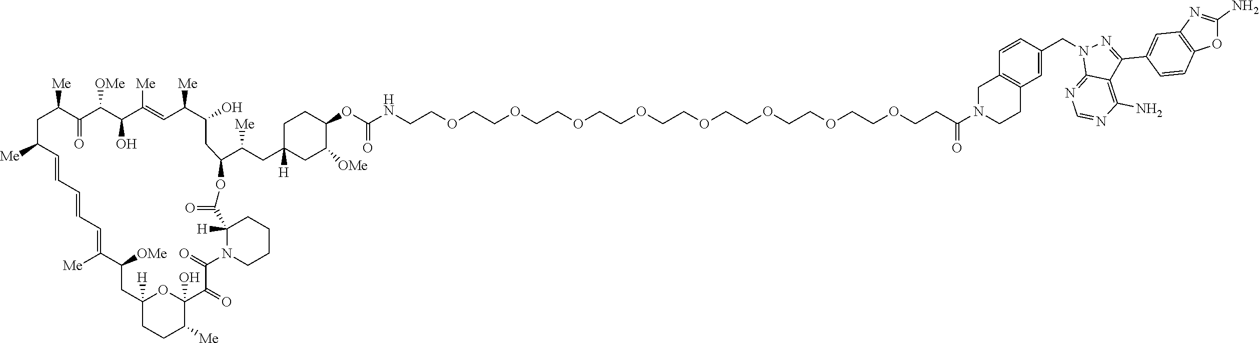 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or an oxepane isomer thereof.
Embodiment I-17. The method of any one of Embodiments I-1 to I-12, wherein the bi-steric inhibitor of mTOR is a compound having the formula

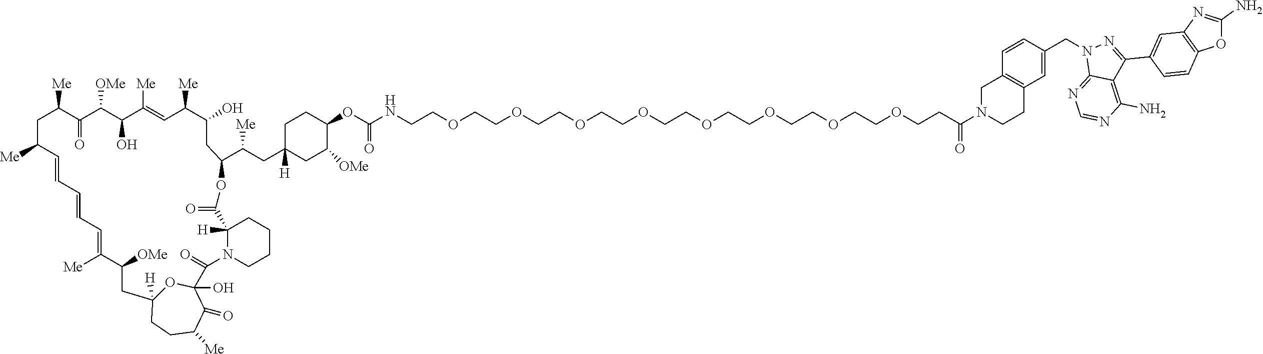 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer thereof.
Embodiment I-18. The method of any one of Embodiments I-1 to I-12, wherein the bi-steric inhibitor of mTOR is a compound having the formula

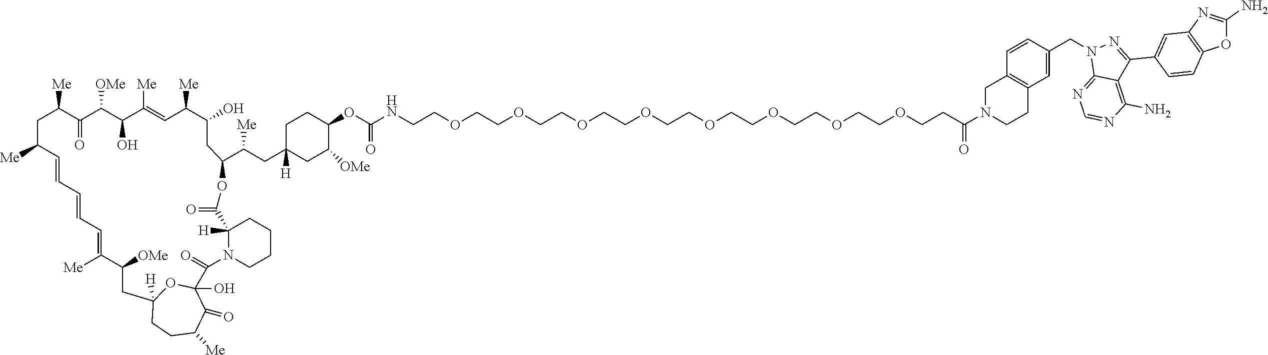 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a tautomer thereof.
Embodiment I-19. The method of any one of Embodiments I-1 to I-12, wherein the bi-steric inhibitor of mTOR is a compound having the formula

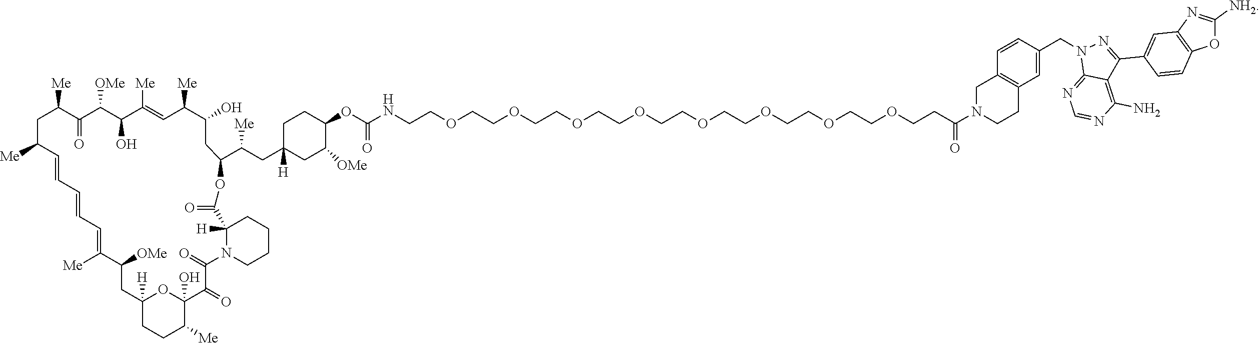 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Embodiment I-20. The method of any one of Embodiments I-1 to I-12, wherein the bi-steric inhibitor of mTOR is a compound having the formula

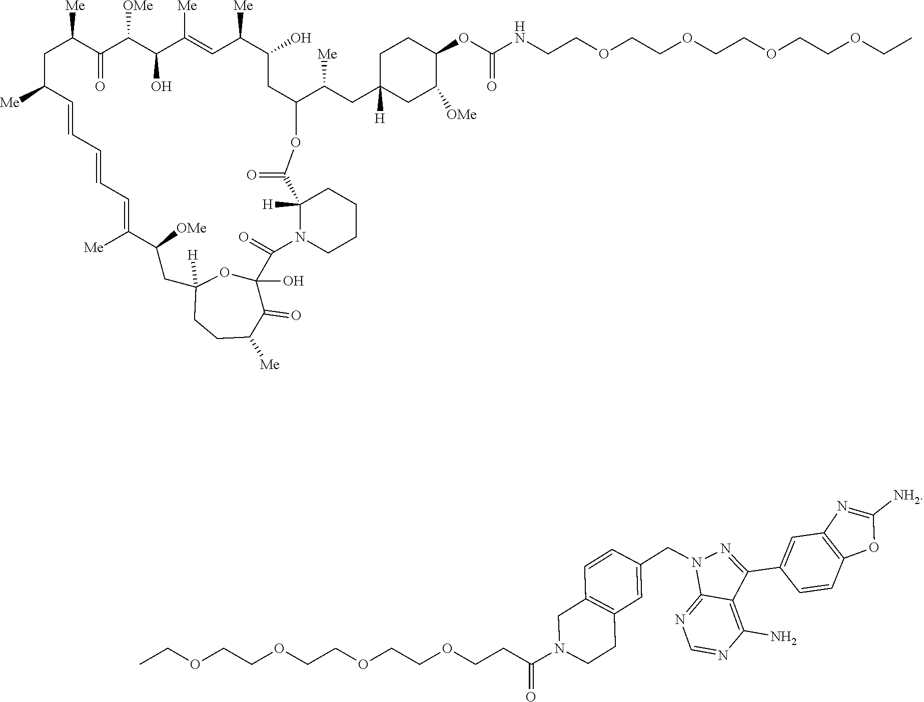

Embodiment I-21. The method of any one of Embodiments I-1 to I-12, wherein the bi-steric inhibitor of mTOR is comprised in a composition comprising a compound having the formula

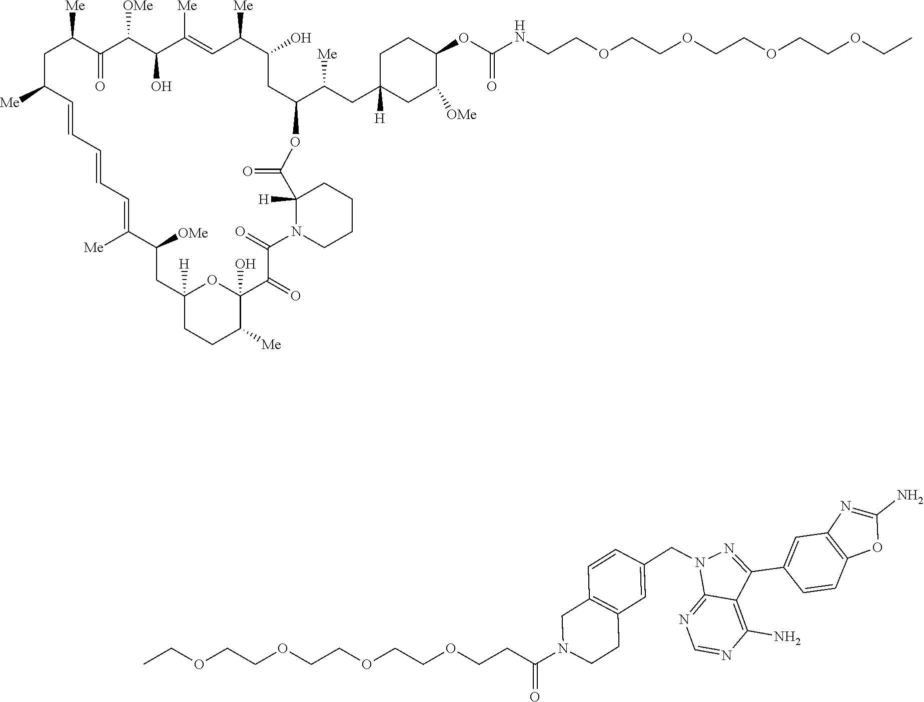

or a stereoisomer or tautomer thereof and a compound having the formula

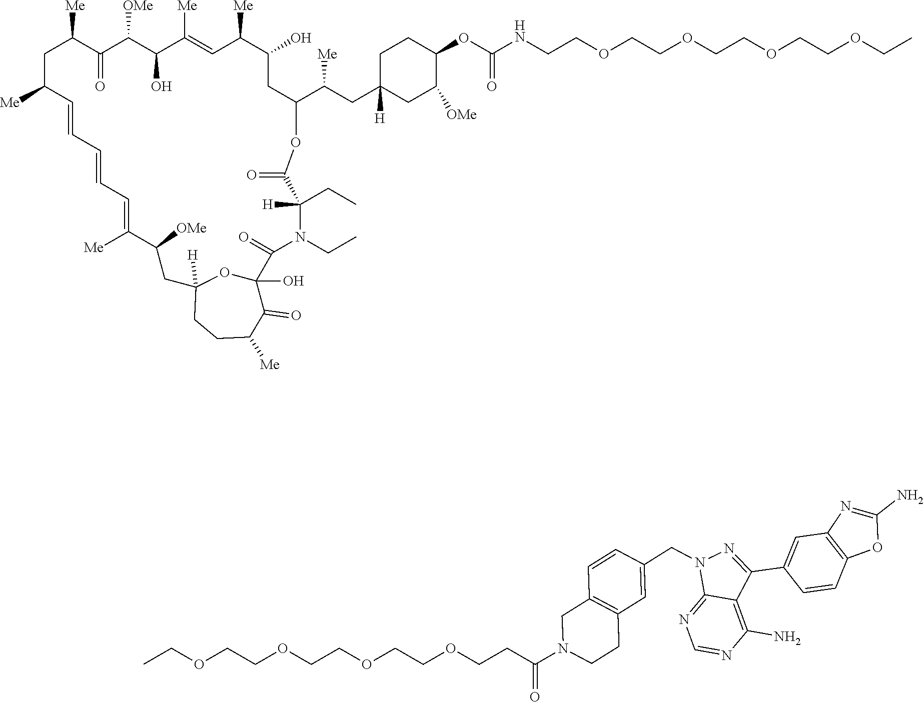

or a stereoisomer or tautomer thereof.
Embodiment I-22. The method of any one of Embodiments I-1 to I-12, wherein the bi-steric inhibitor of mTOR is comprised in a composition comprising a compound having the formula

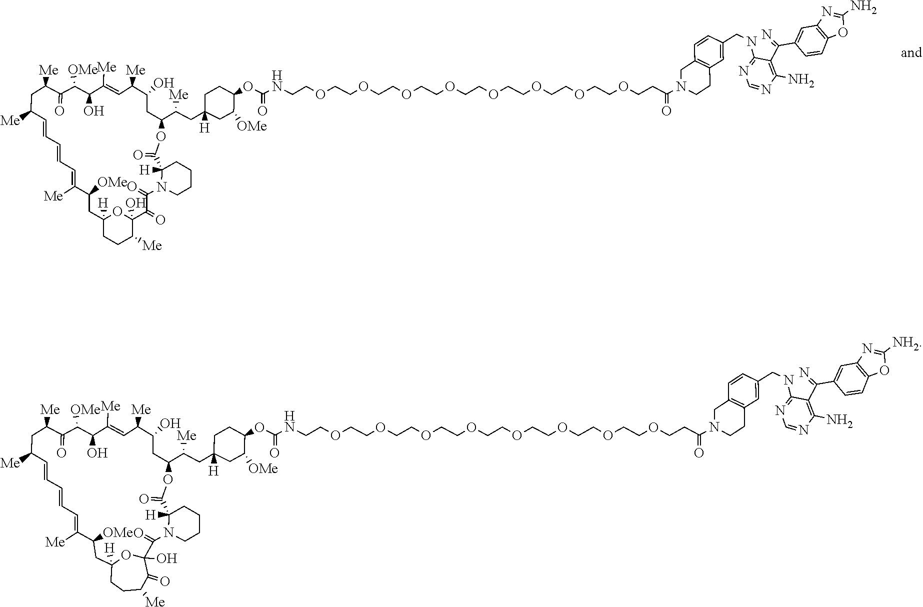

Embodiment I-23. The method of any one of Embodiments I-1 to I-8, Embodiment 11, or Embodiments I-13 to I-22, wherein the RAS inhibitor binds the RAS in its “on” position.
Embodiment I-24. The method of any one of Embodiments I-1 to I-8, Embodiment 11, or Embodiments I-13 to I-23, wherein the RAS inhibitor is a KRAS(ON) inhibitor.
Embodiment I-25. The method of Embodiment I-24, wherein the KRAS(ON) inhibitor is a KRAS^{G12C}(ON) inhibitor.
Embodiment I-26. The method of any one of Embodiments I-1 to I-8, Embodiment I-11, or Embodiments I-13 to I-25, wherein the RAS inhibitor is selected from compounds A1-A741 of Appendix B-1, or a pharmaceutically acceptable salt thereof.
Embodiment I-27. The method of any one of Embodiments I-1 to I-8, Embodiment I-11, or Embodiments I-13 to I-25, wherein the RAS inhibitor is a compound, or a pharmaceutically acceptable salt thereof, of Appendix B-1, Formula VIb,

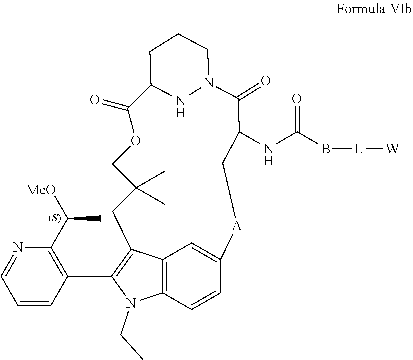

wherein A is a 3 to 6-membered heterocycloalkylene, a phenylene, or a hydroxy-substituted phenylene; B is —CH(C₁-C₆ alkyl)-; L is a linker selected from the following:

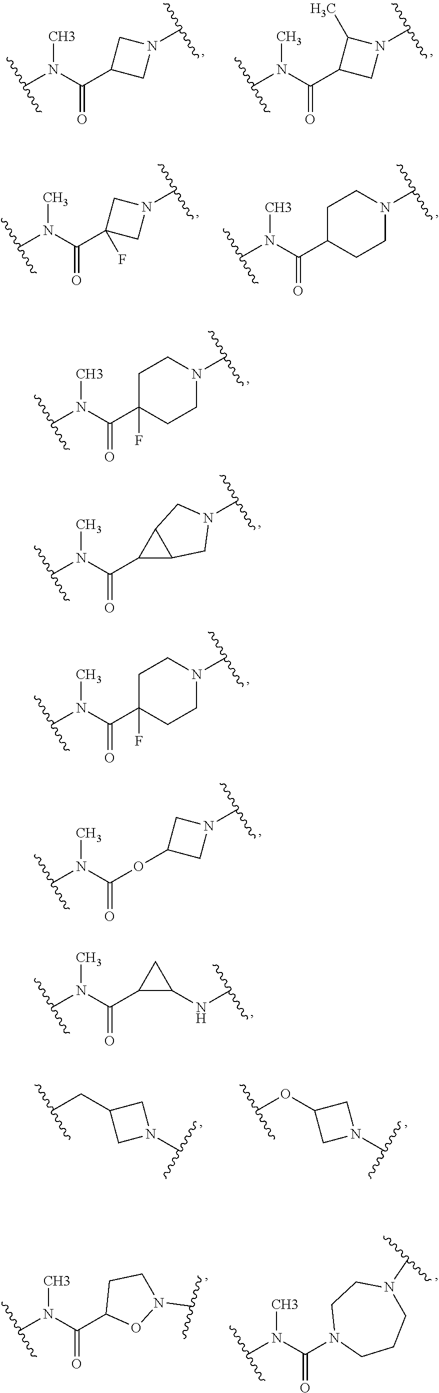

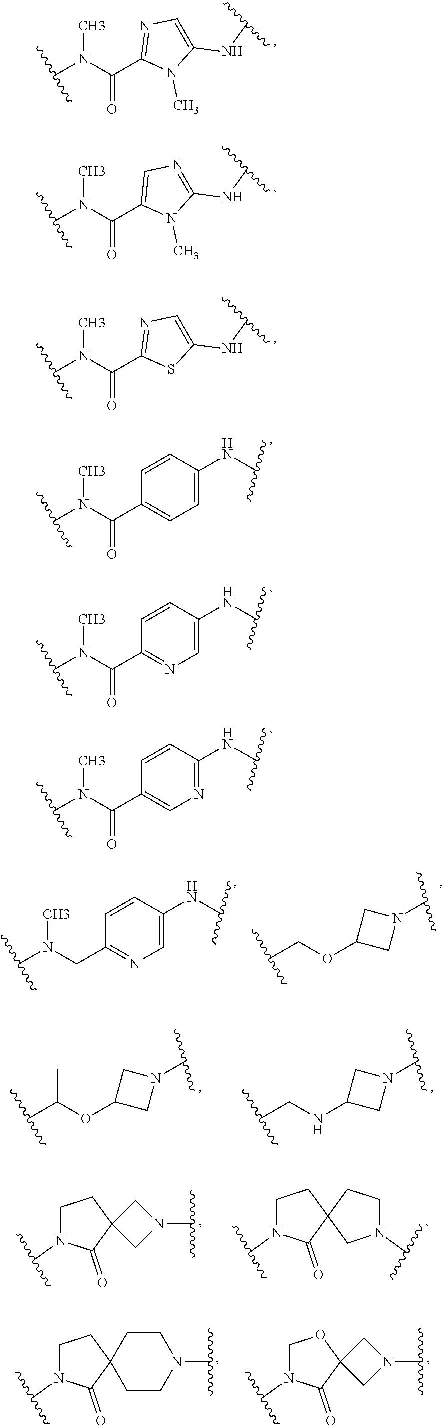

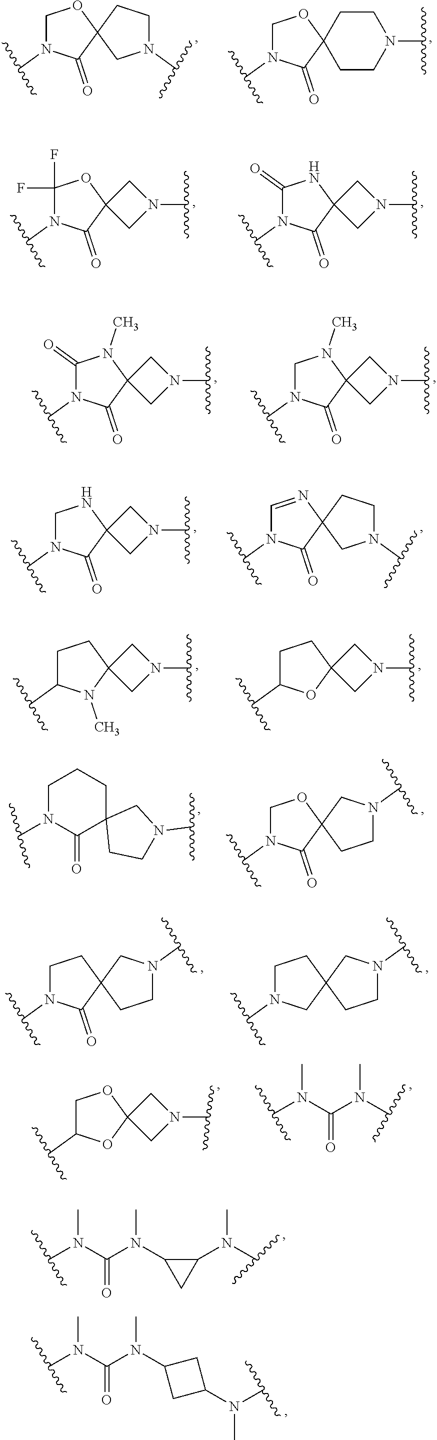

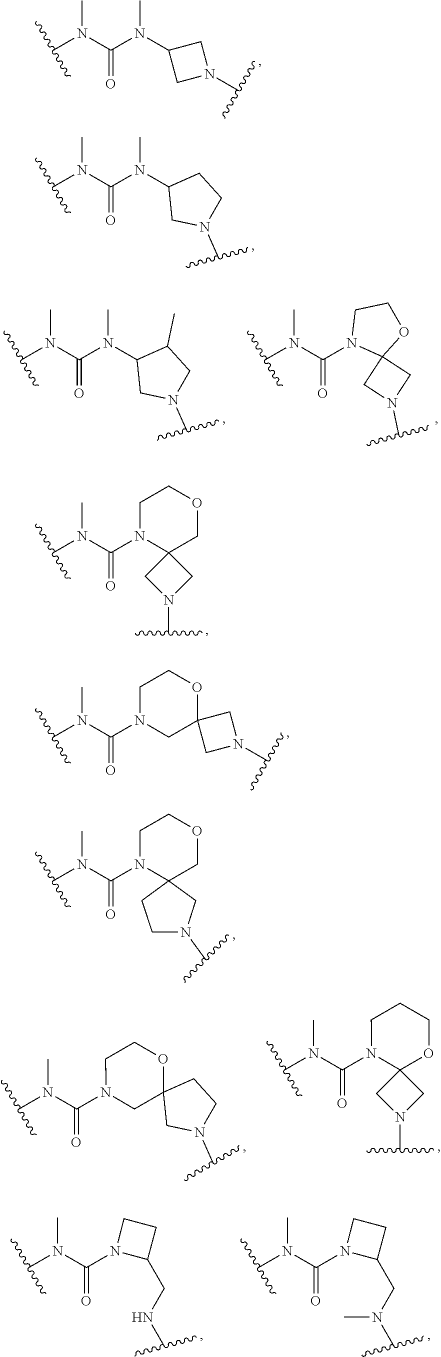

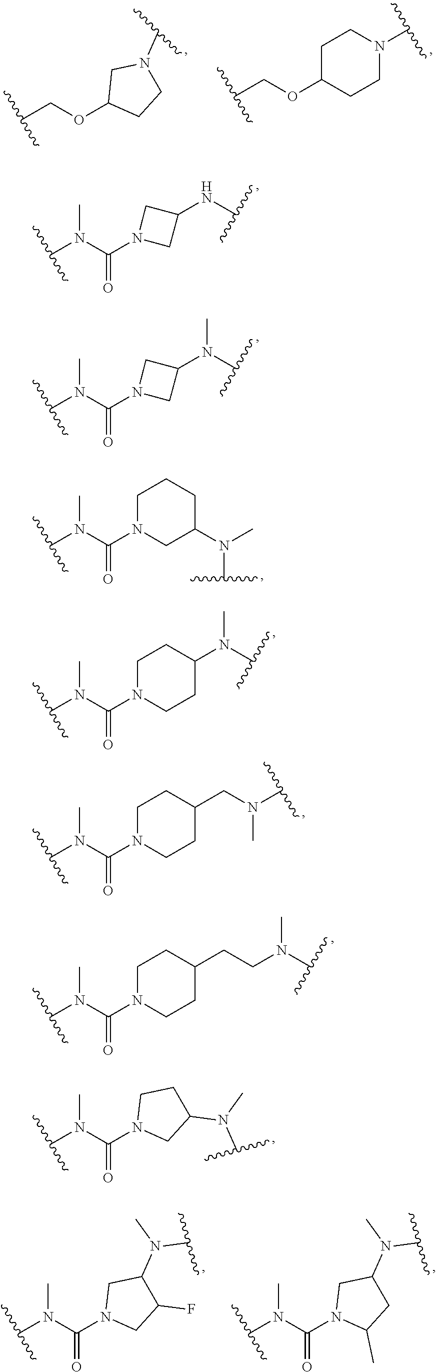

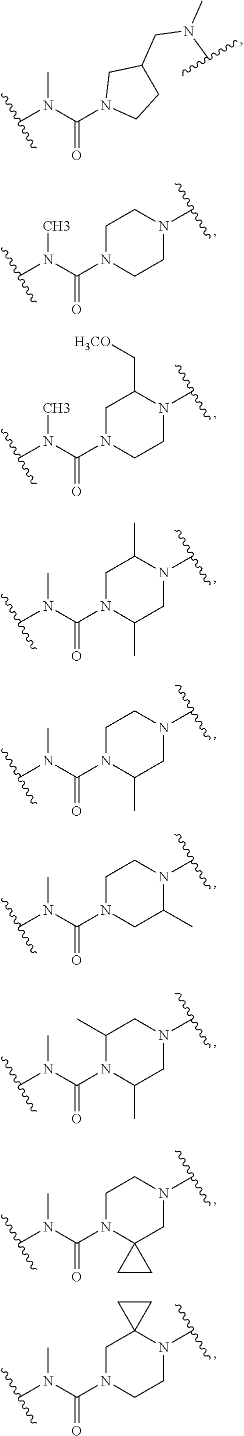

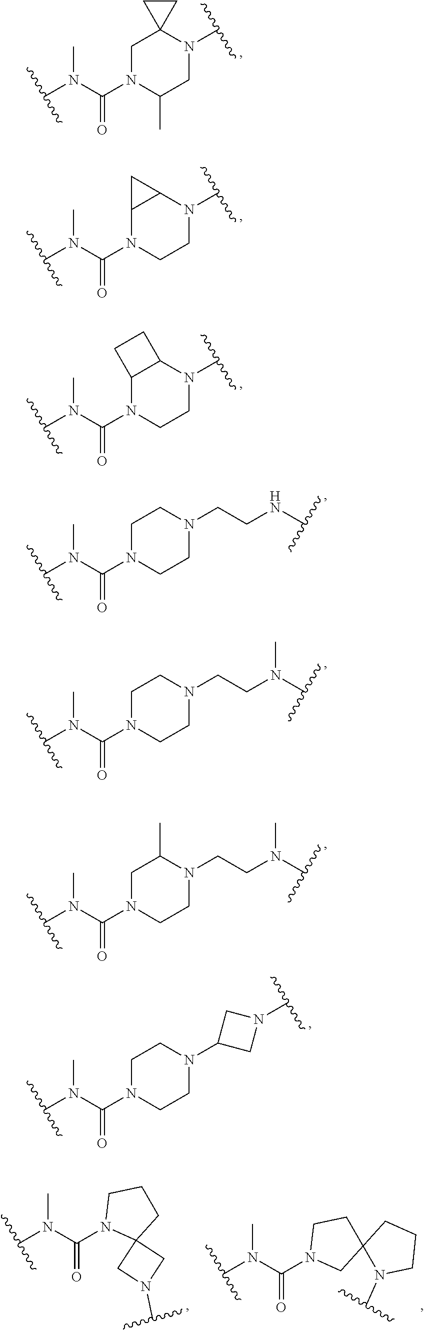

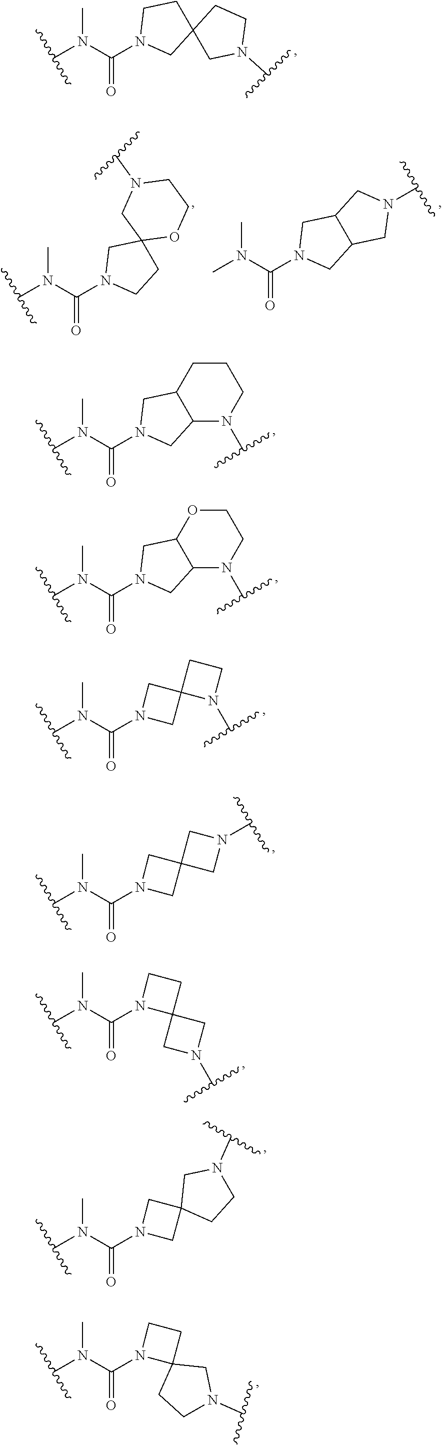

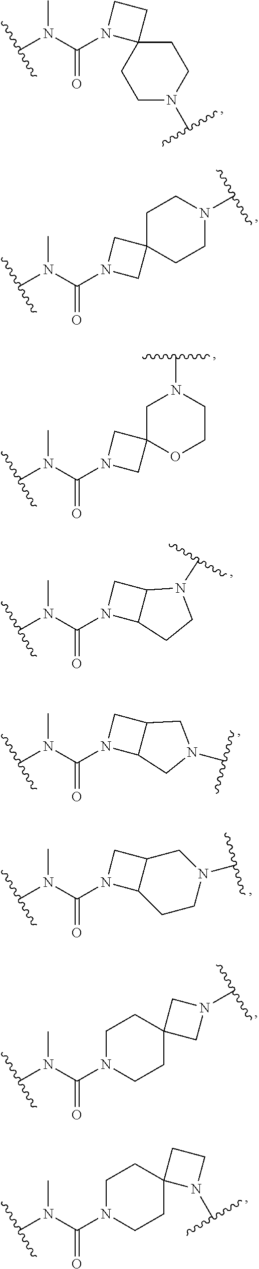

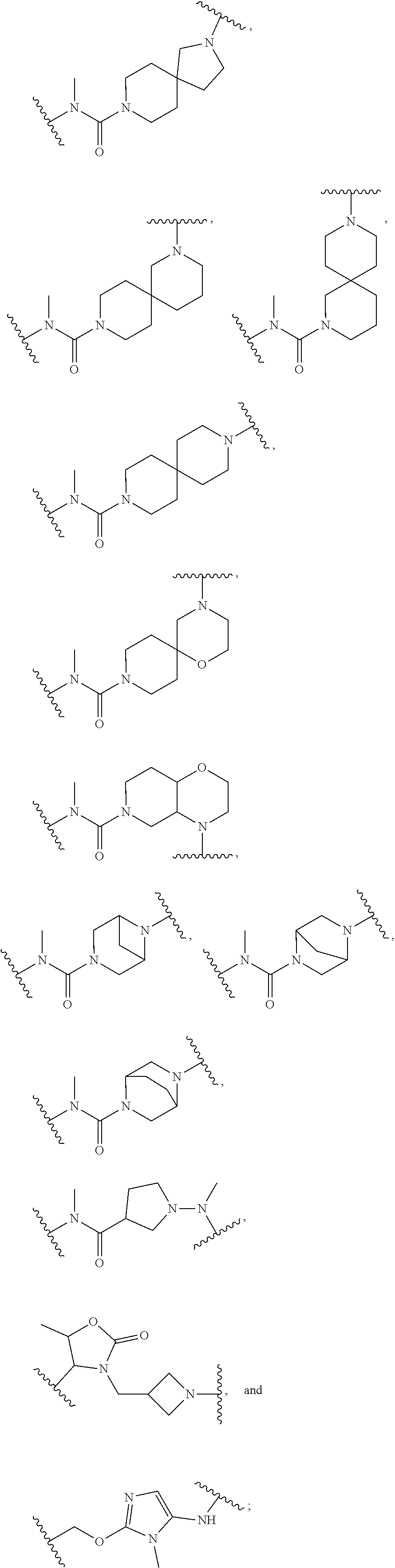

and
W is a cross-linking group selected from the following:

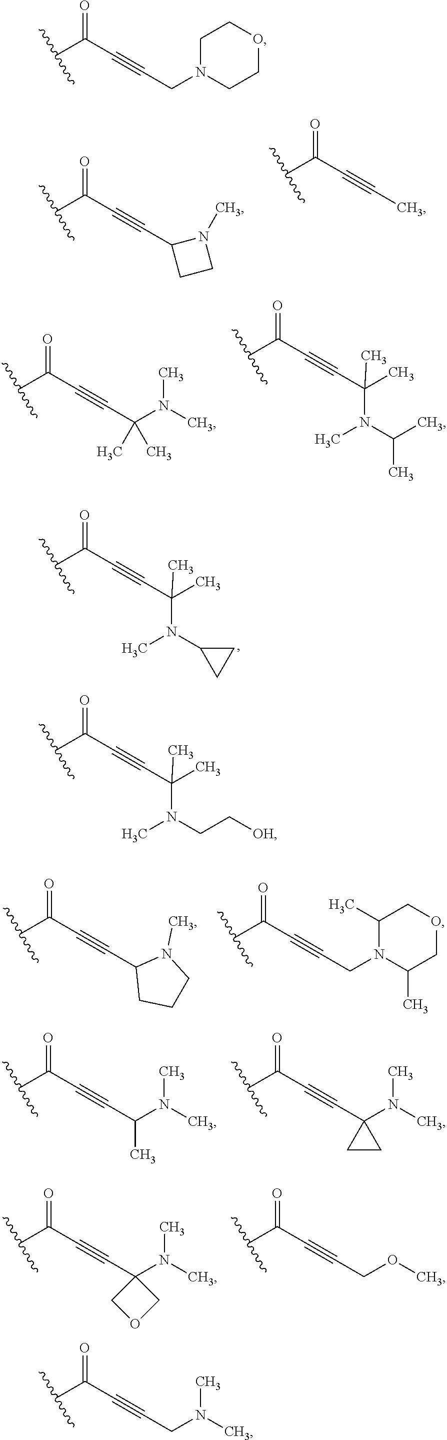

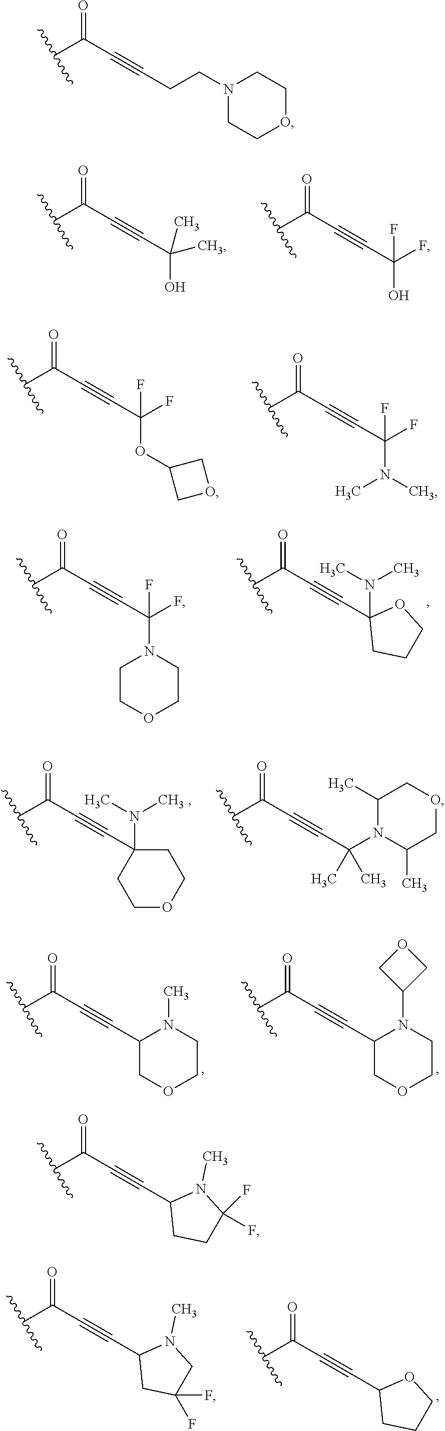

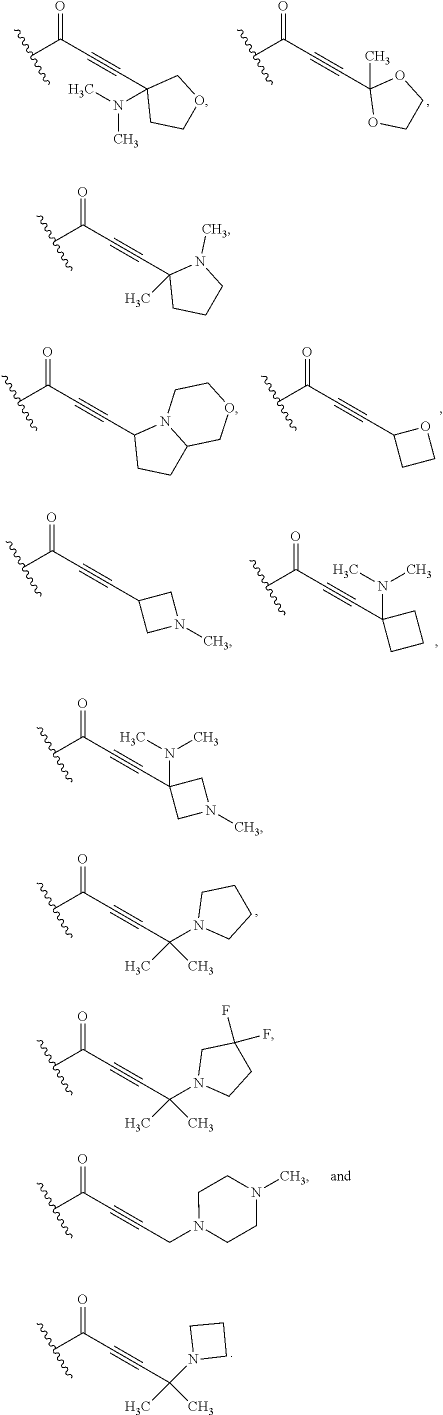

Embodiment I-28. The method of any one of Embodiments I-1 to I-8, Embodiment I-11, or Embodiments I-13 to I-27, wherein the RAS inhibitor is selected from compounds A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof.
Embodiment I-29. The method of any one of Embodiments I-1 to I-8, Embodiment I-11, or Embodiments I-13 to I-28, wherein the RAS inhibitor is Compound A, or a pharmaceutically acceptable salt thereof.
Embodiment I-30. The method of any one of Embodiments I-1 to I-8, Embodiment I-11, or Embodiments I-13 to I-28, wherein the RAS inhibitor is Compound B, or a pharmaceutically acceptable salt thereof.
Embodiment I-31. The method of any one of the preceding Embodiments, wherein the subject is administered the RAS inhibitor to treat or prevent a cancer.
Embodiment I-32. The method of Embodiment I-31, wherein the cancer is a RAS G12C cancer.
Embodiment I-33. The method of Embodiment I-31 or Embodiment I-32, wherein the cancer comprises a KRAS^{G12C} mutation.
Embodiment I-34. The method of any one of Embodiments I-31 to I-33, wherein the cancer comprises co-occurring KRAS^{G12C} and STK11 mutations.
Embodiment I-35. The method of any one of Embodiments I-31 to I-34, wherein the cancer is a Non-Small Cell Lung Cancer (NSCLC).
Embodiment I-36. The method of any one of Embodiments I-31 to I-34, wherein the cancer is a colorectal cancer.
Embodiment I-37. The method of any one of Embodiments I-31 to I-36, wherein the cancer is selected from pancreatic cancer, colorectal cancer, non-small cell lung cancer, squamous cell lung carcinoma, thyroid gland adenocarcinoma, and a hematological cancer.
Embodiment I-38. The method of any one of Embodiments I-31 to I-37, wherein the cancer comprises co-occurring KRAS^{G12C} and PIK3CA^{E545K} mutations.
Embodiment I-39. The method of Embodiment I-37 or Embodiment I-38, wherein the cancer is a colorectal cancer.
Embodiment I-40. The method of any one of Embodiments I-31 to I-39, wherein the method results in tumor regression.
Embodiment I-41. The method of any one of Embodiments I-31 to I-40, wherein the method results in tumor apoptosis.
Embodiment I-42. A method of treating a subject having a cancer comprising administering to the subject an effective amount of a bi-steric inhibitor of mTOR in combination with a RAS inhibitor.
Embodiment I-43. The method of Embodiment I-42, wherein the RAS is selected from KRAS, NRAS, and HRAS.
Embodiment I-44. The method of Embodiment I-42 or Embodiment I-43, wherein the RAS inhibitor targets a specific RAS mutation.
Embodiment I-45. The method of any one of Embodiments I-42 to I-44, wherein the RAS inhibitor targets a KRAS mutation.
Embodiment I-46. The method of any one of Embodiments I-42 to I-45, wherein the RAS inhibitor targets a RAS G12C mutation.
Embodiment I-47. The method of any one of Embodiments I-42 to I-46, wherein the RAS inhibitor targets the KRAS^{G12C} mutation.
Embodiment I-48. The method of any one of Embodiments I-42 to I-47, wherein the RAS inhibitor binds the RAS in its “off” position.
Embodiment I-49. The method of any one of Embodiments I-42 to I-48, wherein the RAS inhibitor is a KRAS(OFF) inhibitor.
Embodiment I-50. The method of any one of Embodiments I-42 to I-45 or Embodiments I-48 or Embodiment I-49, wherein the KRAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation.
Embodiment I-51. The method of any one of Embodiments I-42 to I-50, wherein the KRAS inhibitor is selected from AMG 510, MRTX849, JDQ443 and MRTX1133, or a pharmaceutically acceptable salt thereof.
Embodiment I-52. The method of one of Embodiments I-42 to I-51, wherein the bi-steric inhibitor of mTOR is RM-006, also known as RMC-6272, or RMC-5552, or a pharmaceutically acceptable salt thereof.
Embodiment I-53. The method of any one of Embodiments I-42 to I-51, wherein the bi-steric inhibitor of mTOR is a compound having the formula

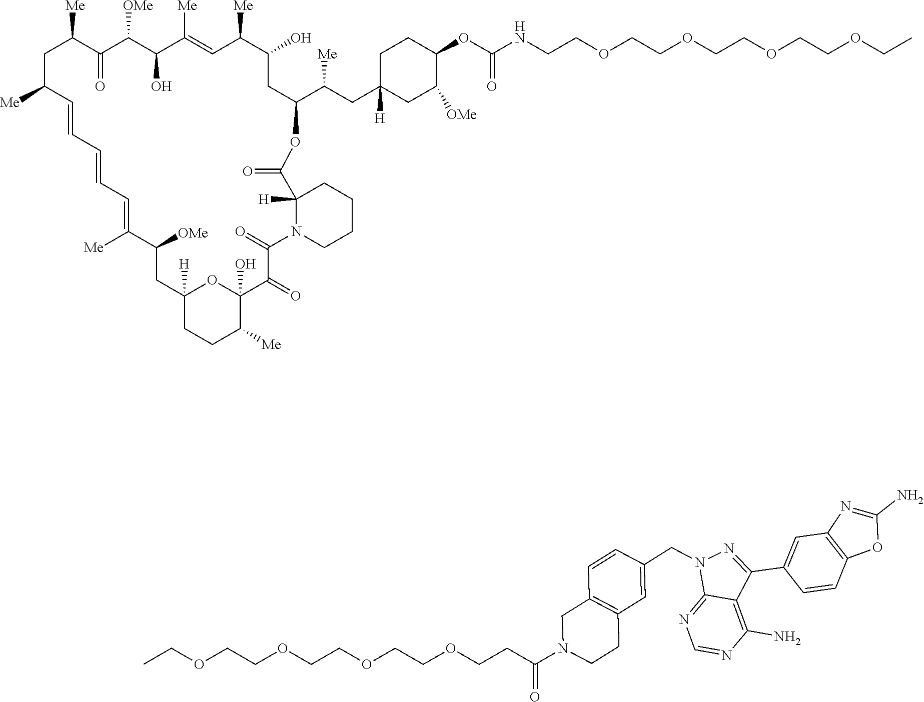

or a stereoisomer thereof.
Embodiment I-54. The method of any one of Embodiments I-42 to I-51, wherein the bi-steric inhibitor of mTOR is a compound having the formula

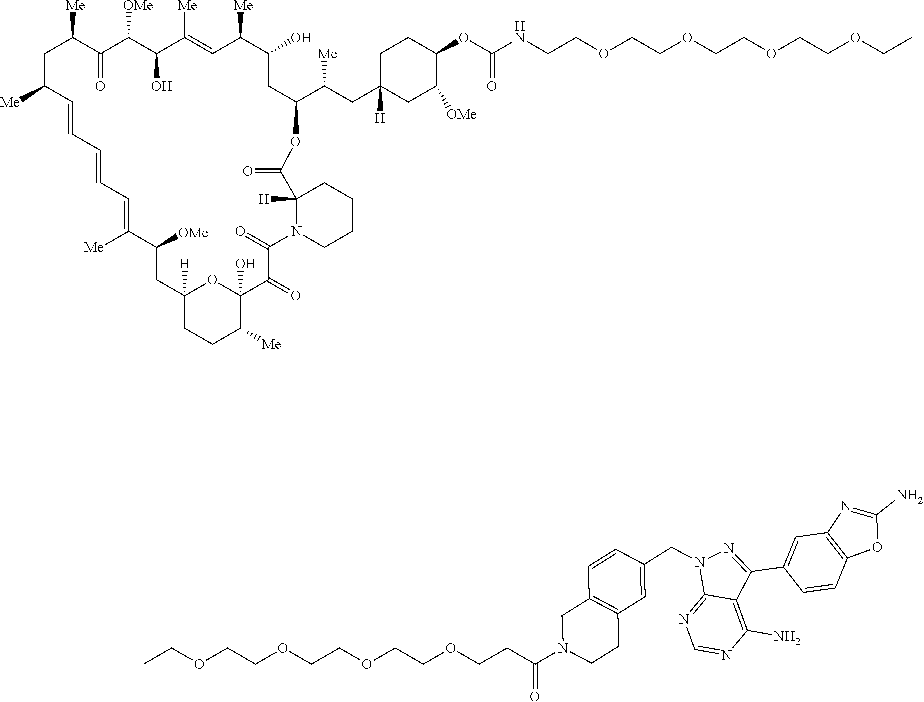

or a tautomer thereof.
Embodiment I-55. The method of any one of Embodiments I-42 to I-51, wherein the bi-steric inhibitor of mTOR is a compound having the formula

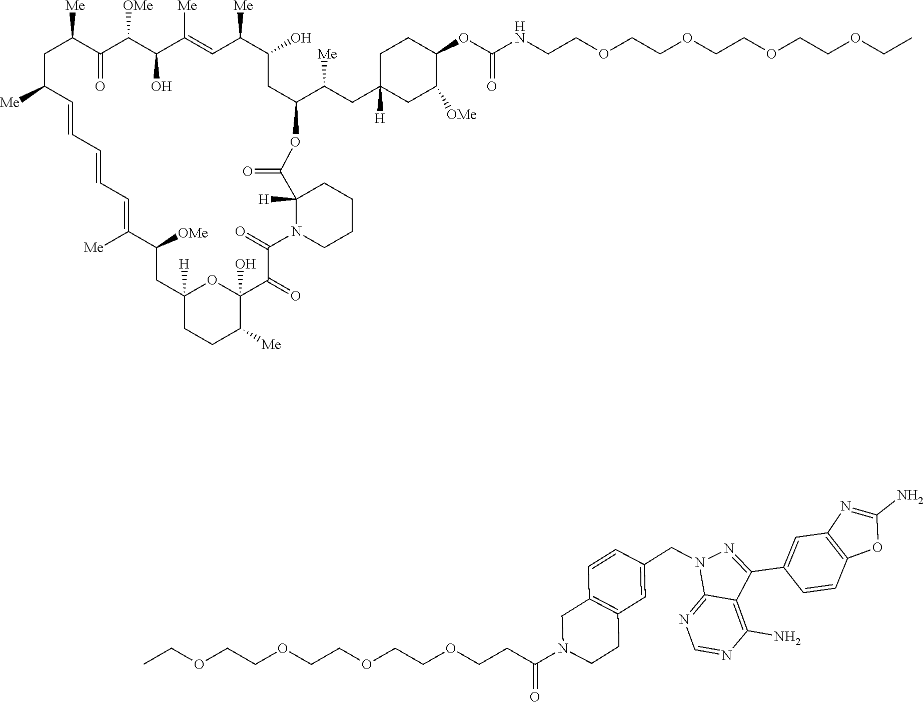

or an oxepane isomer thereof.
Embodiment I-56. The method of any one of Embodiments I-42 to I-51, wherein the bi-steric inhibitor of mTOR is a compound having the formula

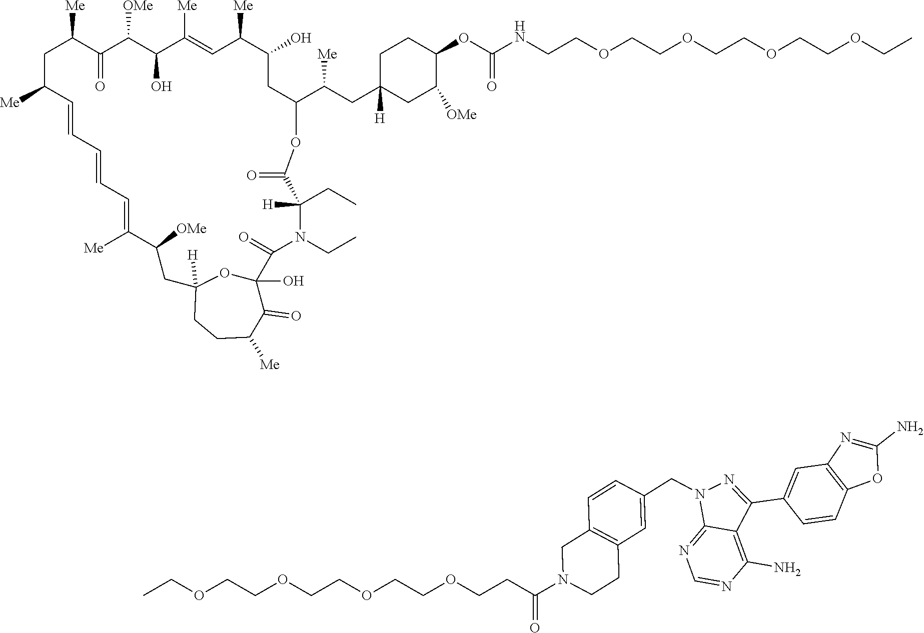

or a stereoisomer thereof.
Embodiment I-57. The method of any one of Embodiments I-42 to I-51, wherein the bi-steric inhibitor of mTOR is a compound having the formula

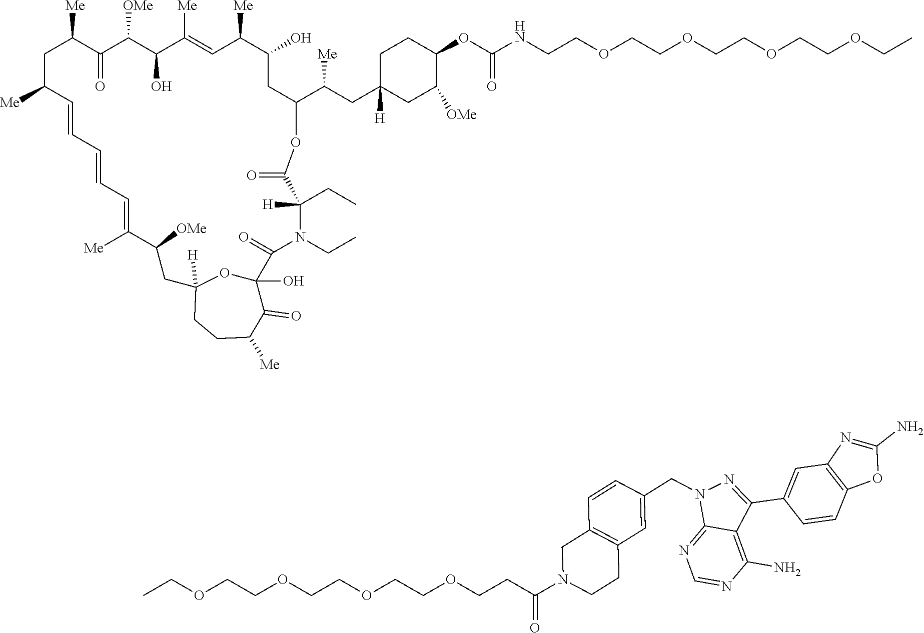

or a tautomer thereof.
Embodiment I-58. The method of any one of Embodiments I-42 to I-51, wherein the bi-steric inhibitor of mTOR is a compound having the formula

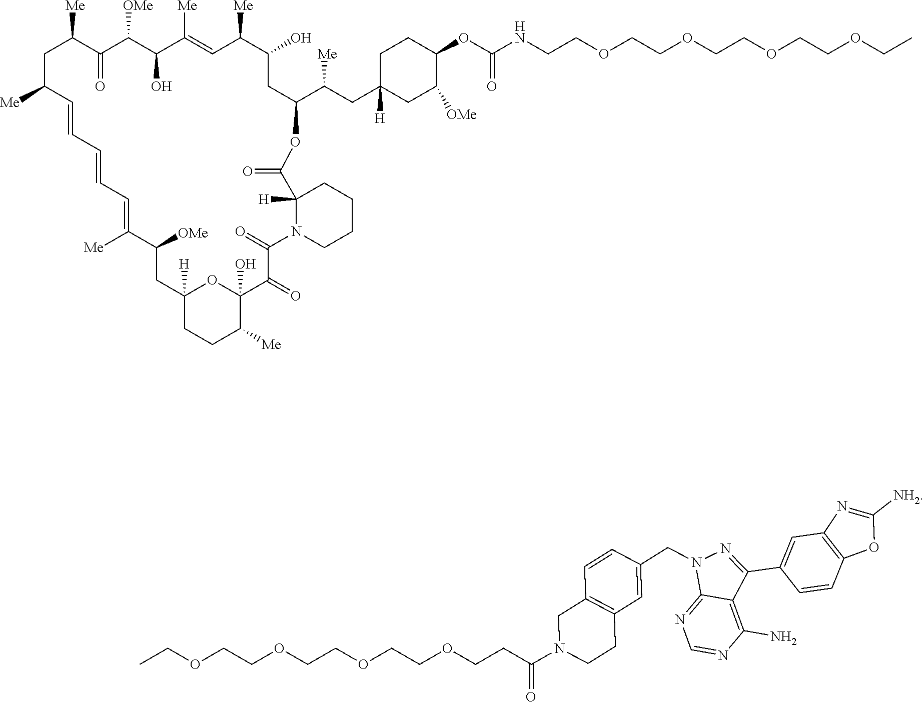

Embodiment I-59. The method of any one of Embodiments I-42 to I-51, wherein the bi-steric inhibitor of mTOR is a compound having the formula

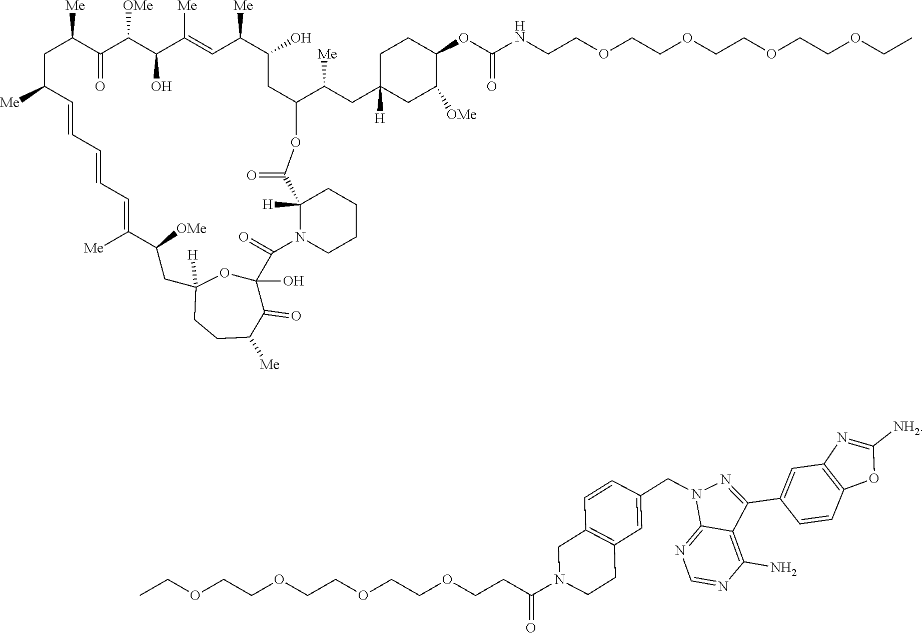

Embodiment I-60. The method of any one of Embodiments I-42 to I-51, wherein the bi-steric inhibitor of mTOR is comprised in a composition comprising a compound having the formula

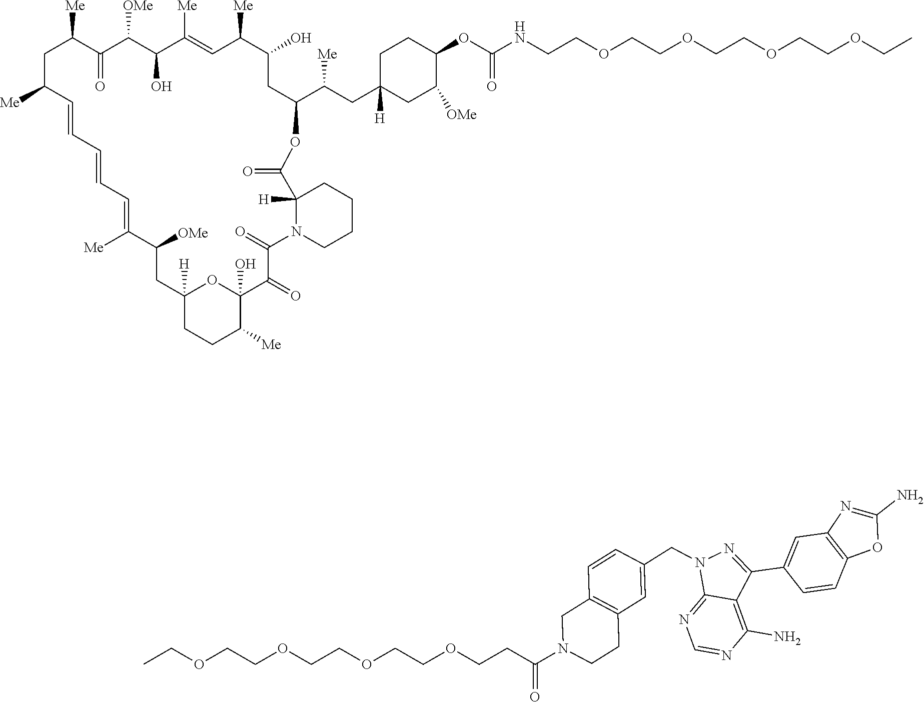

or a stereoisomer or tautomer thereof and a compound having the formula

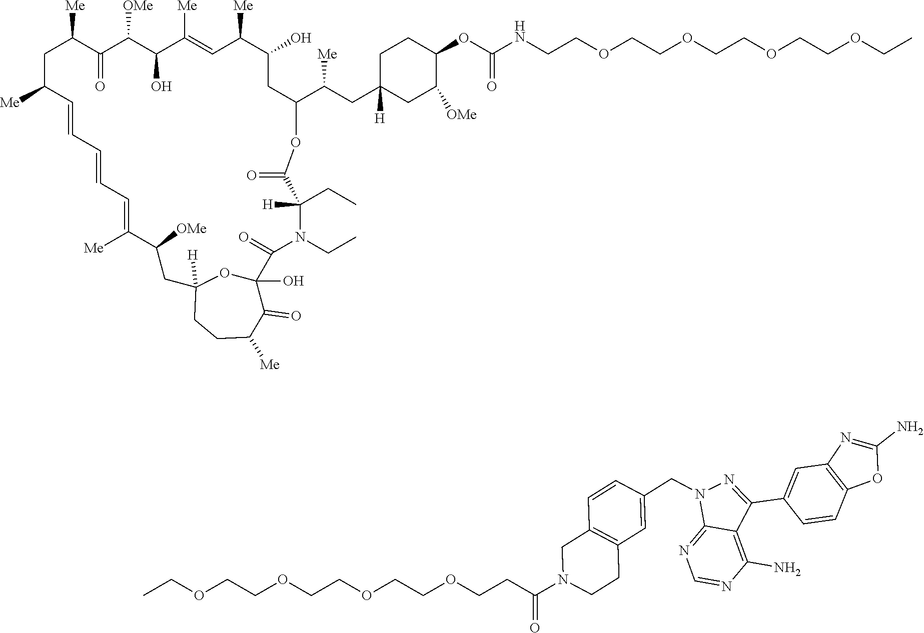

or a stereoisomer or tautomer thereof.
Embodiment I-61. The method of any one of Embodiments I-42 to I-51, wherein the bi-steric inhibitor of mTOR is comprised in a composition comprising a compound having the formula

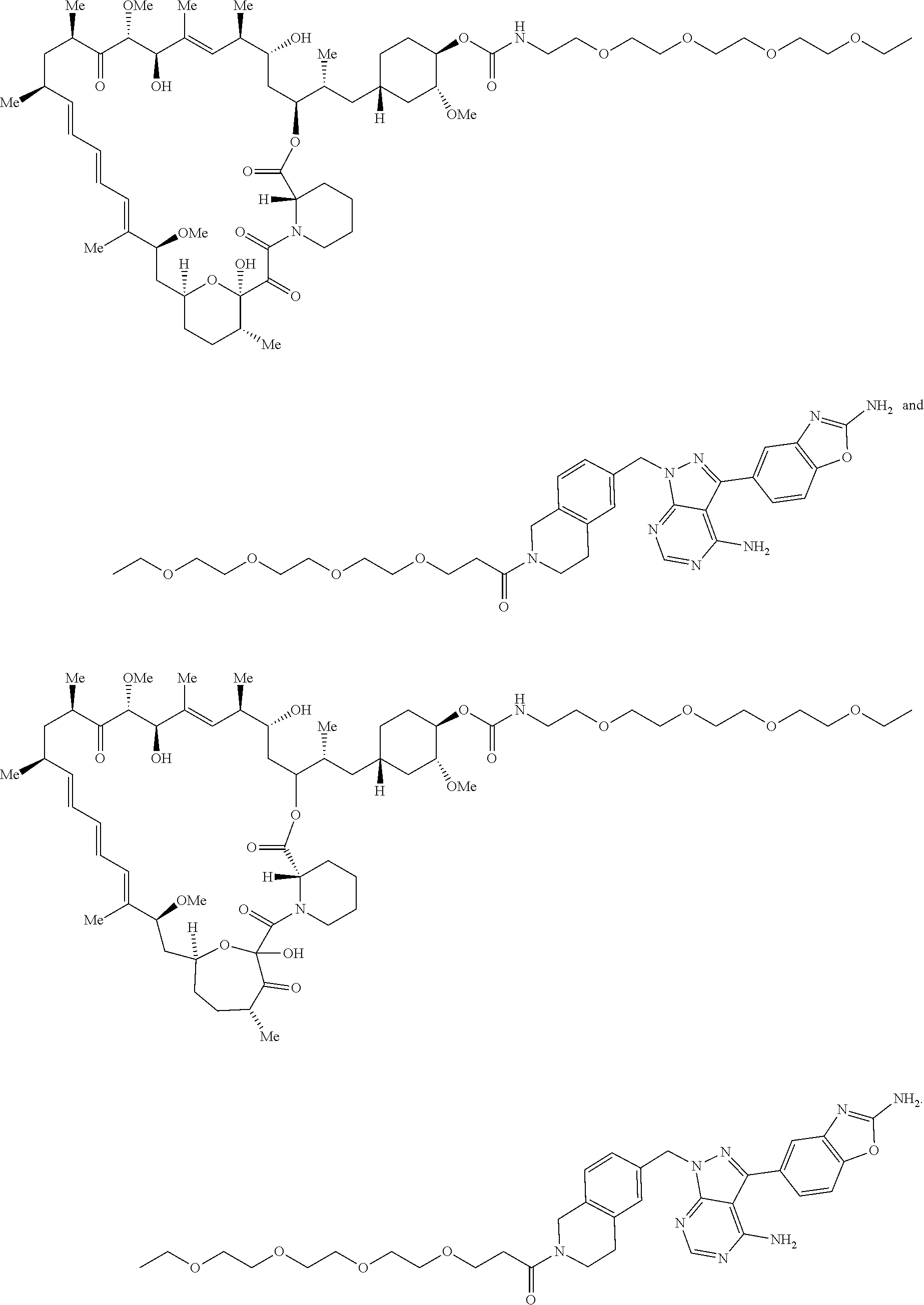

Embodiment I-62. The method of any one of Embodiments I-42 to I-47, Embodiment I-50, or Embodiments I-52 to I-61, wherein the RAS inhibitor binds the RAS in its “on” position.
Embodiment I-63. The method of Embodiment I-62, wherein the RAS inhibitor is a KRAS(ON) inhibitor.
Embodiment I-64. The method of Embodiment I-63, wherein the KRAS(ON) inhibitor is a KRAS^{G12C}(ON) inhibitor.
Embodiment I-65. The method of any one of Embodiments I-42 to I-47, Embodiment I-50, or Embodiments I-52 to I-64, wherein the RAS inhibitor is selected from compounds A1-A741 of Appendix B-1, or a pharmaceutically acceptable salt thereof.
Embodiment I-66. The method of any one of Embodiments I-42 to I-47, Embodiment I-50 or Embodiments I-52 to I-64, wherein the RAS inhibitor is a compound, or a pharmaceutically acceptable salt thereof, of Appendix B-1, Formula VIb,

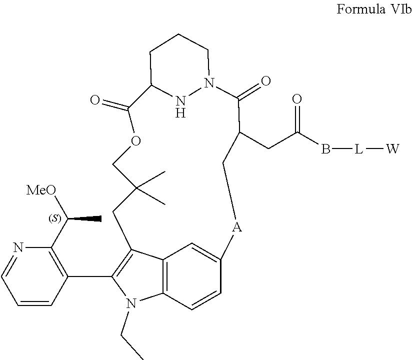

wherein A is a 3 to 6-membered heterocycloalkylene, a phenylene, or a hydroxy-substituted phenylene; B is —CH(C₁-C₆ alkyl)-; L is a linker selected from the following:

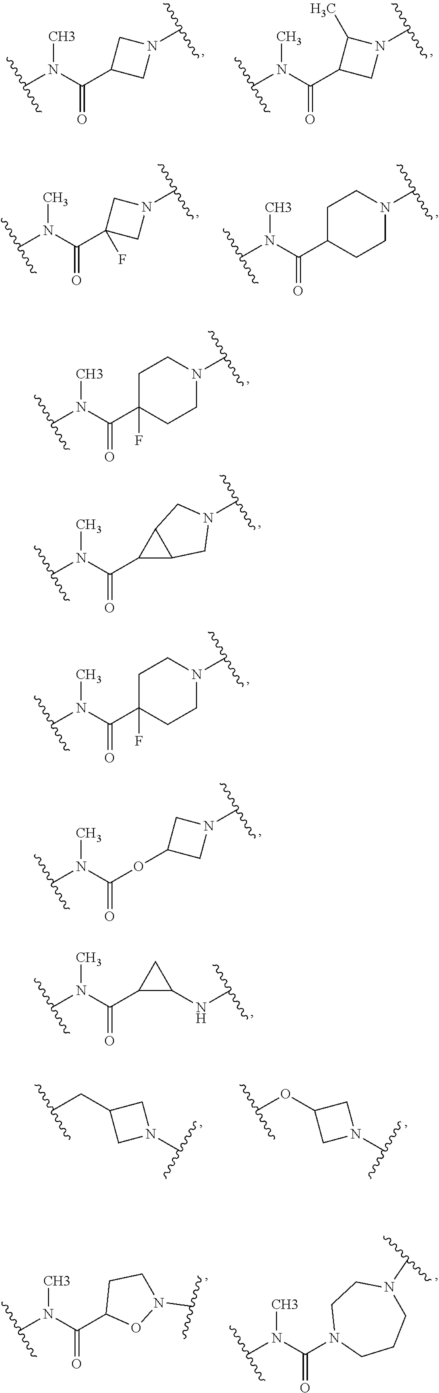

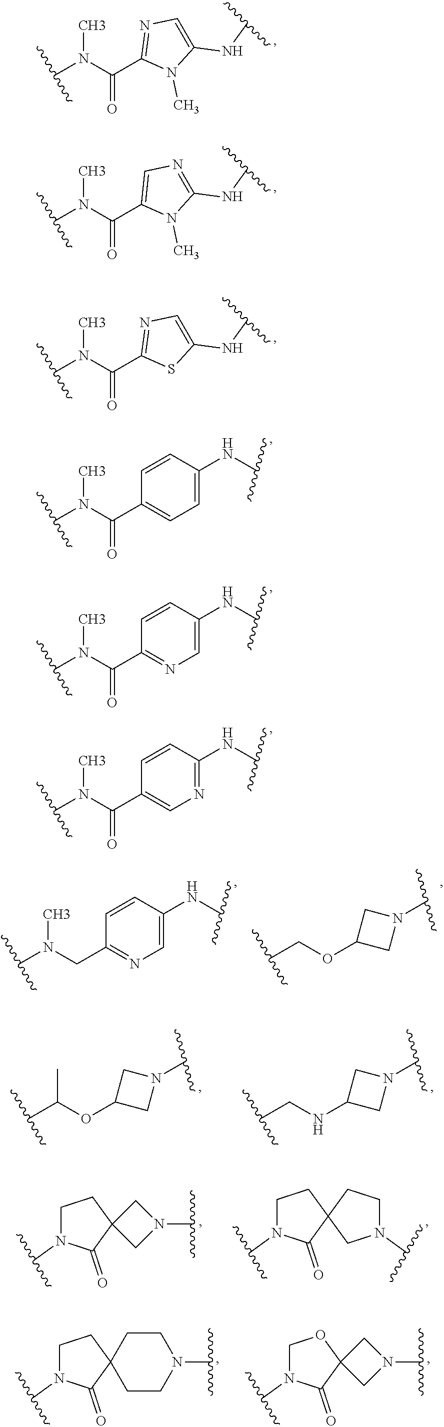

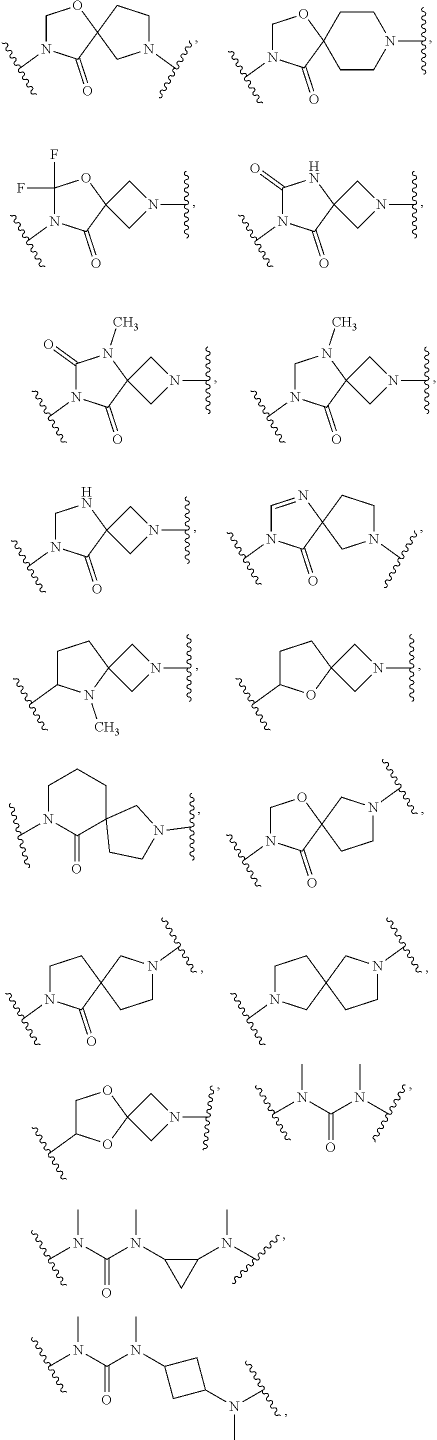

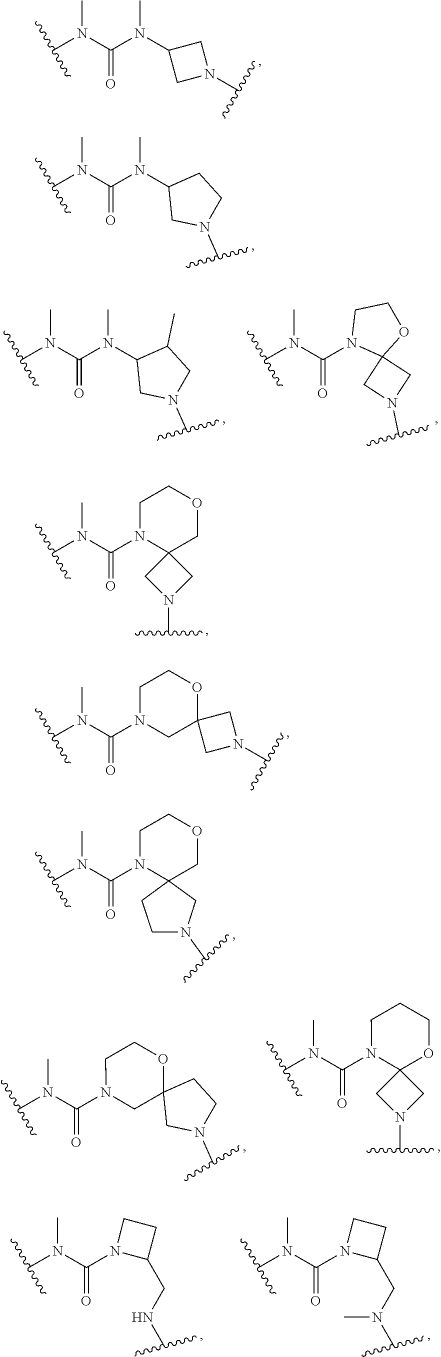

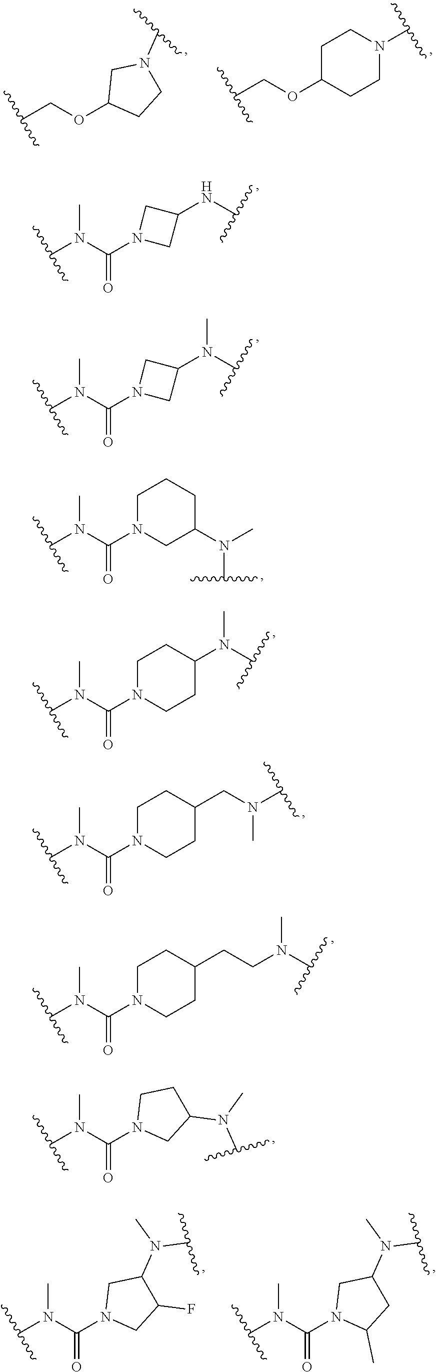

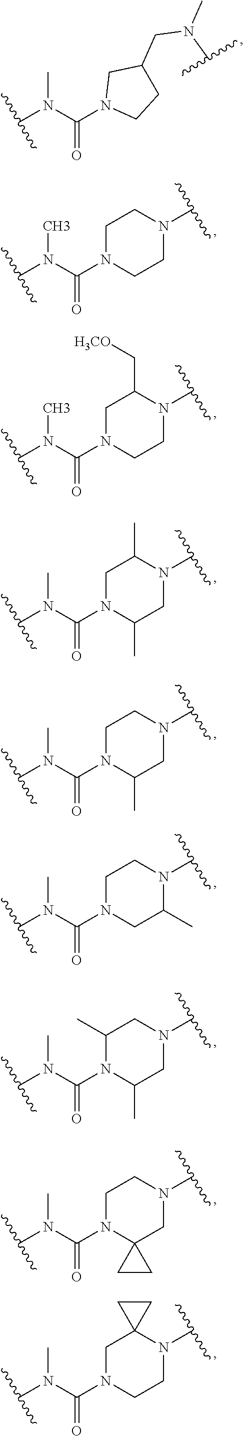

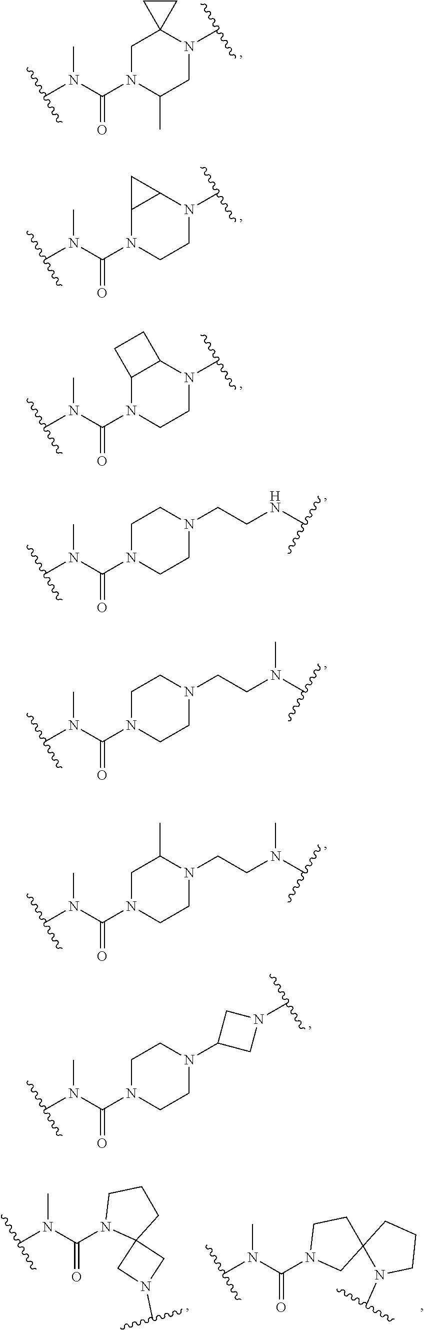

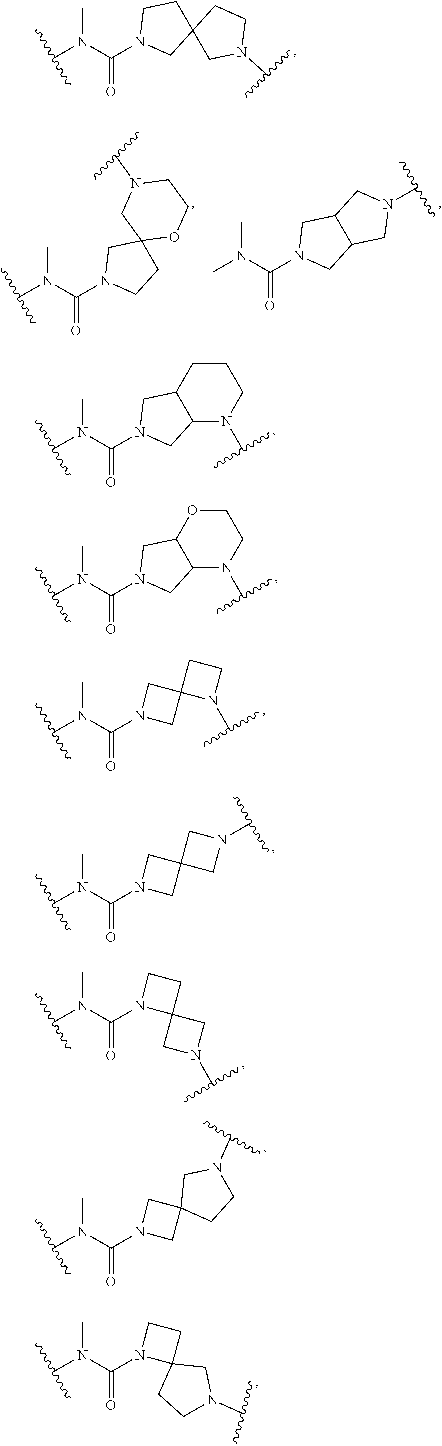

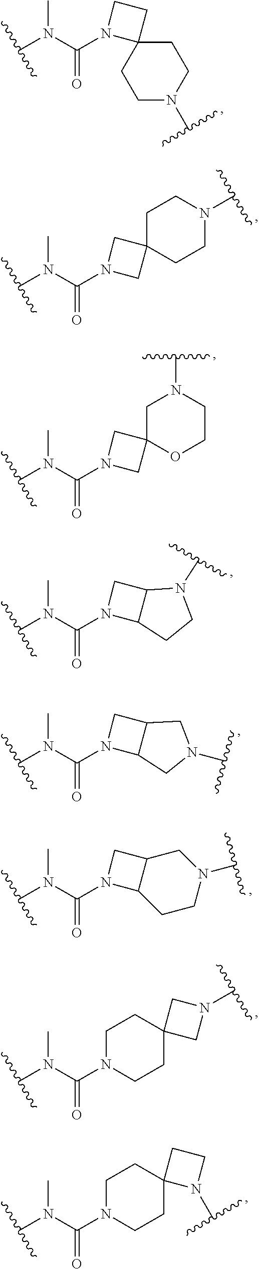

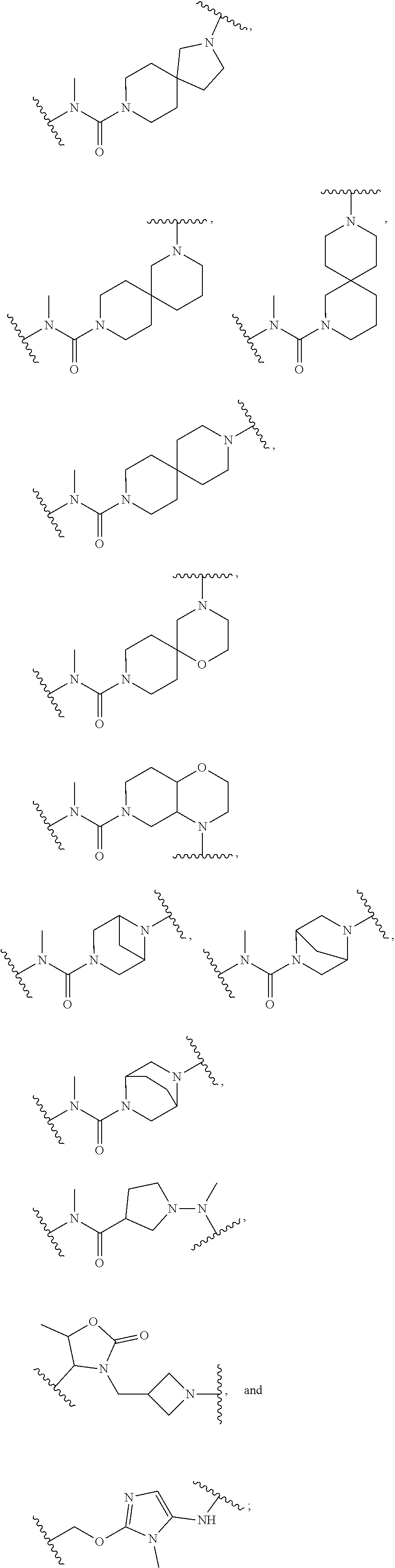

and
W is a cross-linking group selected from the following:

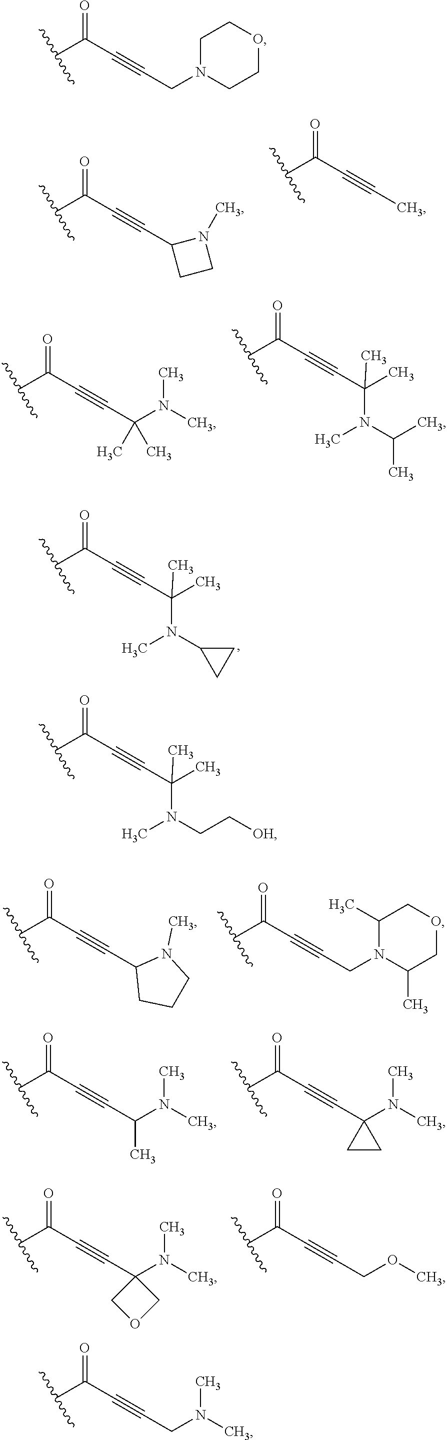

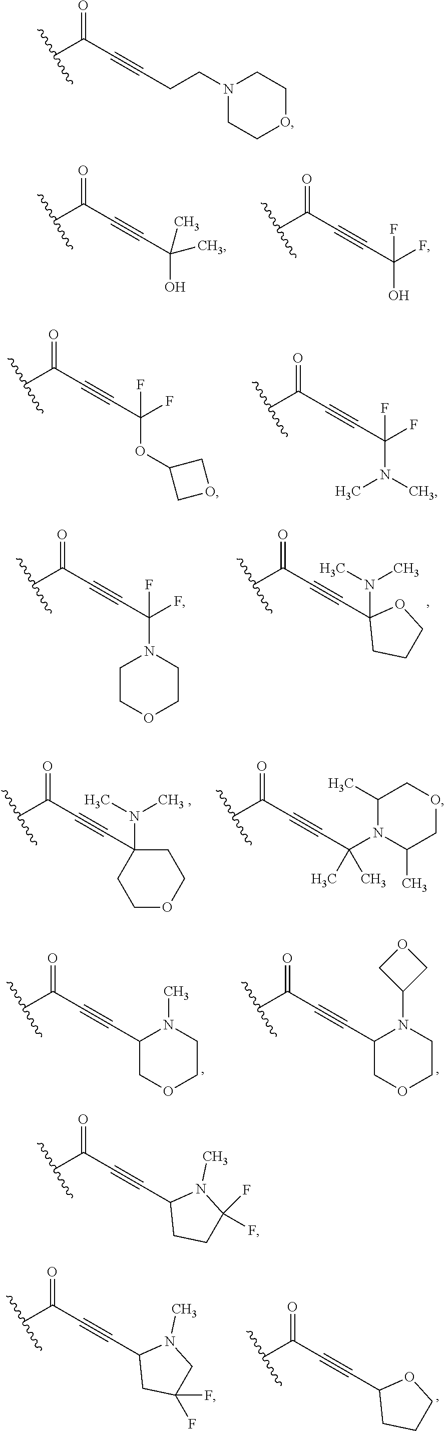

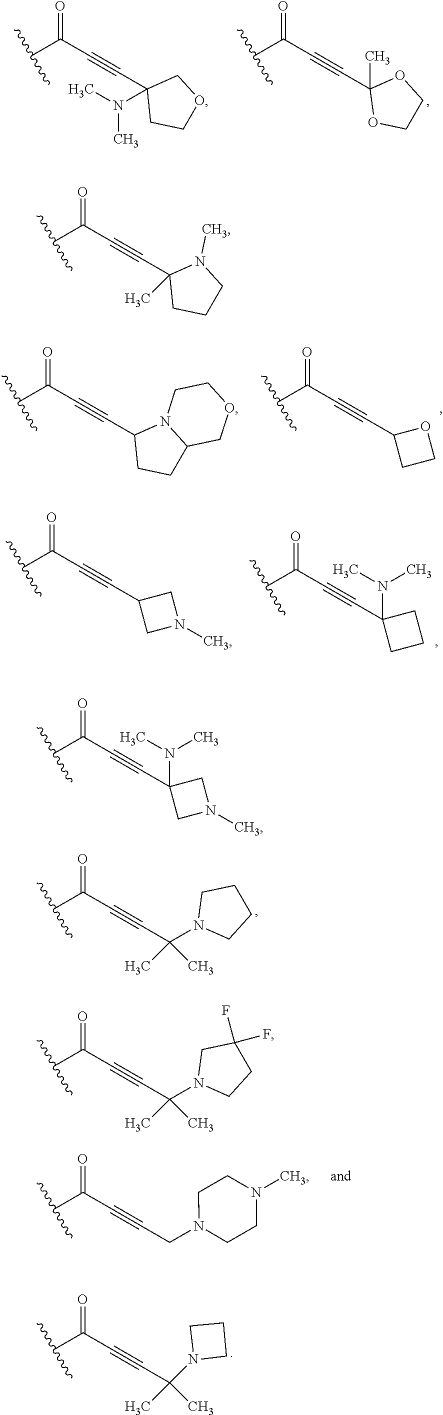

Embodiment I-67. The method of any one of Embodiments I-42 to I-47, Embodiment I-50 or Embodiments I-52 to I-66, wherein the RAS inhibitor is selected from compounds A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof.
Embodiment I-68. The method of any one of Embodiments I-42 to I-47, Embodiments I-50 or Embodiments I-52 to I-67, wherein the RAS inhibitor is Compound A, or a pharmaceutically acceptable salt thereof.
Embodiment I-69. The method of any one of Embodiments I-42 to I-47, Embodiments I-50 or Embodiments I-52 to I-67, wherein the RAS inhibitor is Compound B, or a pharmaceutically acceptable salt thereof.
Embodiment I-70. The method of any one of Embodiments I-42 to I-49 or Embodiments I-51 to I-69, wherein the cancer is a RAS G12C cancer.
Embodiment I-71. The method of any one of Embodiments I-42 to I-70, wherein the cancer comprises a KRAS^{G12C} mutation.
Embodiment I-72. The method of any one of Embodiments I-42 to I-71, wherein the cancer comprises co-occurring KRAS^{G12C} and STK11 mutations.
Embodiment I-73. The method of any one of c Embodiments I-42 to I-71, wherein the cancer is a Non-Small Cell Lung Cancer (NSCLC).
Embodiment I-74. The method of any one of Embodiments I-42 to I-72, wherein the cancer is a colorectal cancer.
Embodiment I-75. The method of any one of Embodiments I-42 to I-74, wherein the cancer is selected from pancreatic cancer, colorectal cancer, non-small cell lung cancer, squamous cell lung carcinoma, thyroid gland adenocarcinoma, and a hematological cancer.
Embodiment I-76. The method of any one of Embodiments I-42 to I-75, wherein the cancer comprises co-occurring KRAS^{G12C} and PIK3CA^{E545K} mutations.
Embodiment I-77. The method of any one of Embodiments I-42 to I-72 or Embodiments I-74 to 1-76, wherein the cancer is a colorectal cancer.
Embodiment I-78. The method of any one of Embodiments I-42 to I-77, wherein the method results in tumor regression.
Embodiment I-79. The method of any one of Embodiments I-42 to I-78, wherein the method results in tumor apoptosis.
Embodiment I-80. A method of inducing apoptosis of a tumor cell comprising contacting the tumor cell with an effective amount of a bi-steric inhibitor of mTOR in combination with a RAS inhibitor, wherein the effective amount is an amount effective to induce apoptosis of the tumor cell.
Embodiment I-81. The method of Embodiment I-80, wherein the RAS is selected from KRAS, NRAS, and HRAS.
Embodiment I-82. The method of Embodiment I-80 or Embodiment I-81, wherein the RAS inhibitor targets a specific RAS mutation.
Embodiment I-83. The method of any one of Embodiments I-80 to I-82, wherein the RAS inhibitor targets a KRAS mutation.
Embodiment I-84. The method of any one of Embodiments I-80 to I-83, wherein the RAS inhibitor targets a RAS G12C mutation.
Embodiment I-85. The method of any one of Embodiments I-80 to I-84, wherein the RAS inhibitor targets the KRAS^{G12C} mutation.
Embodiment I-86. The method of any one of Embodiments I-80 to I-85, wherein the RAS inhibitor binds the RAS in its “off” position.
Embodiment I-87. The method of any one of Embodiments I-80 to I-86, wherein the RAS inhibitor is a KRAS(OFF) inhibitor.
Embodiment I-88. The method of any one of Embodiments I-80 to I-84 or Embodiments I-85 to I-87, wherein the KRAS inhibitor targets a KRAS mutation selected from a KRAS^{G12A} mutation, a KRAS^{G12D} mutation, a KRAS^{G12F} mutation, a KRAS^{G12I} mutation, a KRAS^{G12L} mutation, a KRAS^{G12R} mutation, a KRAS^{G12S} mutation, a KRAS^{G12V} mutation, and a KRAS^{G12Y} mutation.
Embodiment I-89. The method of any one of Embodiments I-80 to I-88, wherein the KRAS inhibitor is selected from AMG 510, MRTX849, JDQ443 and MRTX1133, or a pharmaceutically acceptable salt thereof.
Embodiment I-90. The method of one of Embodiments I-80 to I-89, wherein the inhibitor of mTOR is RM-006, also known as RMC-6272, or RMC-5552, or a pharmaceutically acceptable salt thereof.
Embodiment I-91. The method of any one of Embodiments I-80 to I-89, wherein the bi-steric inhibitor of mTOR is a compound having the formula

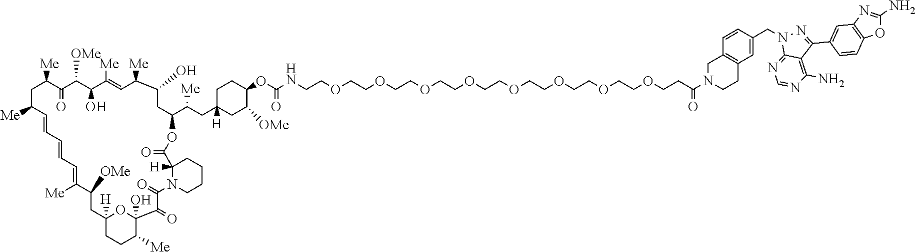 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer thereof.
Embodiment I-92. The method of any one of Embodiments I-80 to I-88, wherein the bi-steric inhibitor of mTOR is a compound having the formula

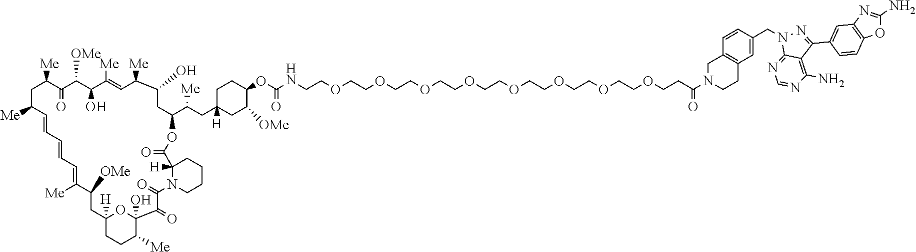 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a tautomer thereof.
Embodiment I-93. The method of any one of Embodiments I-80 to I-88, wherein the bi-steric inhibitor of mTOR is a compound having the formula

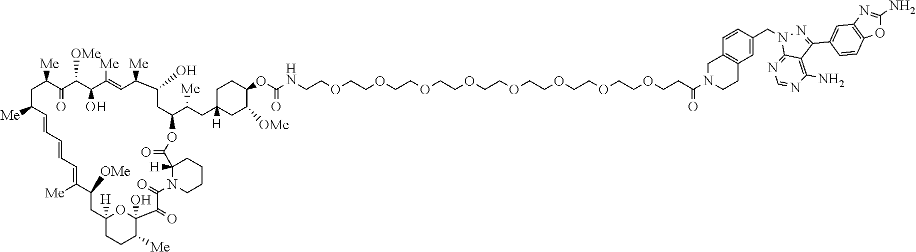 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or an oxepane isomer thereof.
Embodiment I-94. The method of any one of Embodiments I-80 to I-88, wherein the bi-steric inhibitor of mTOR is a compound having the formula

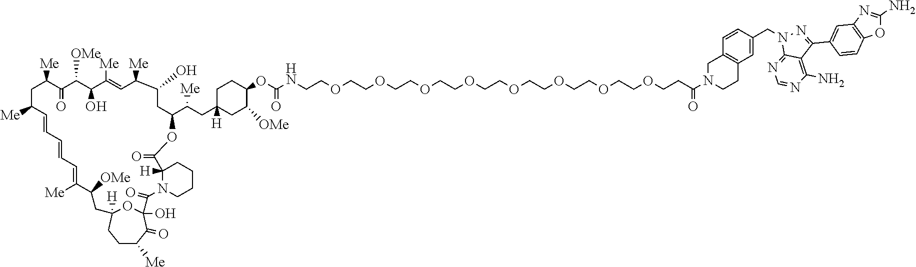 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer thereof.
Embodiment I-95. The method of any one of Embodiments I-80 to I-88, wherein the bi-steric inhibitor of mTOR is a compound having the formula

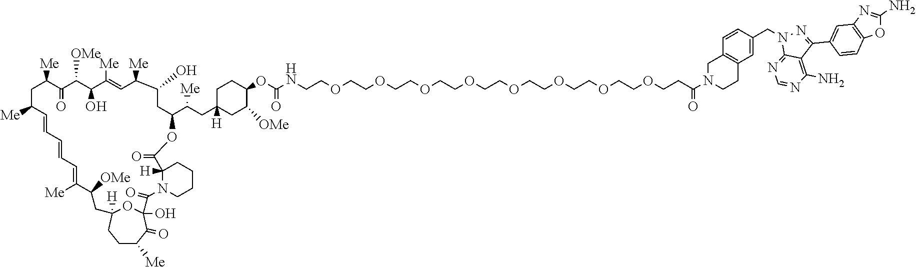 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a tautomer thereof.
Embodiment I-96. The method of any one of Embodiments I-80 to I-88, wherein the bi-steric inhibitor of mTOR is a compound having the formula

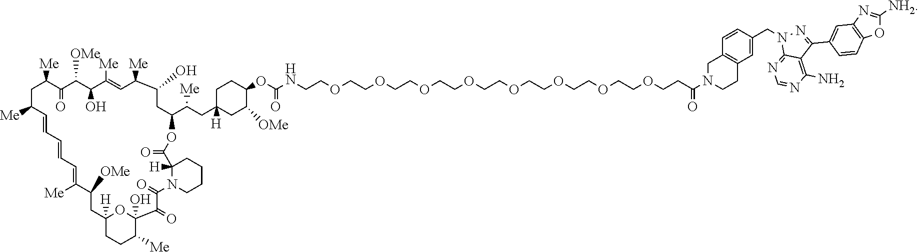 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Embodiment I-97. The method of any one of Embodiments I-80 to I-88, wherein the bi-steric inhibitor of mTOR is a compound having the formula

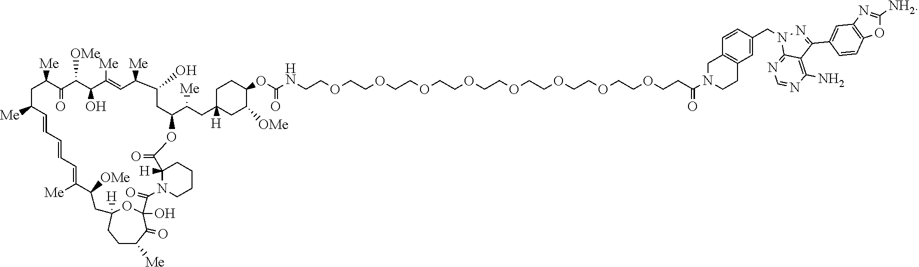 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Embodiment I-98. The method of any one of Embodiments I-80 to I-88, wherein the bi-steric inhibitor of mTOR is comprised in a composition comprising a compound having the formula

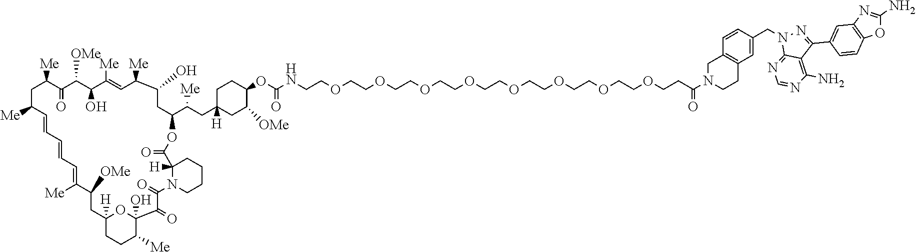 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer or tautomer thereof and a compound having the formula

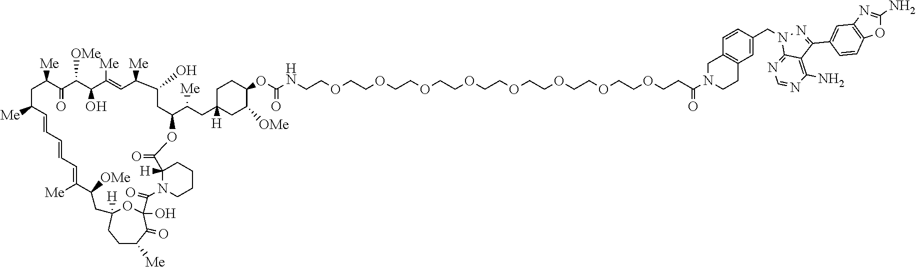 [SMILES: CO[C@H]1C[C@@H]2CC[C@@H](C)C(=O)C(O)(O2)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a stereoisomer or tautomer thereof.
Embodiment I-99. The method of any one of Embodiments I-80 to I-88, wherein the bi-steric inhibitor of mTOR is comprised in a composition comprising a compound having the formula

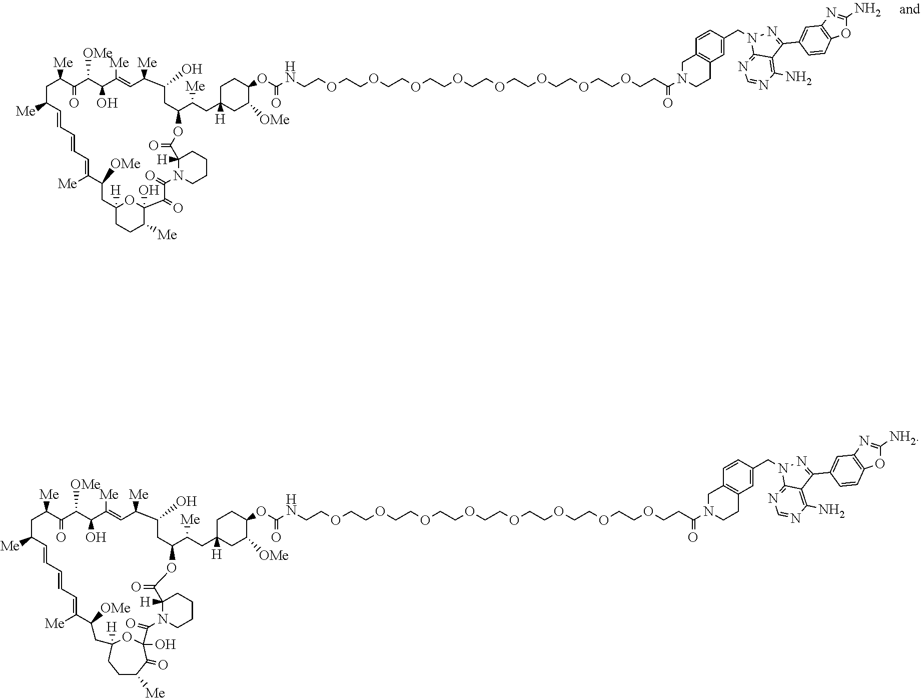

Embodiment I-100. The method of any one of Embodiments I-80 to I-85, Embodiment I-88 or Embodiments I-90 to I-99, wherein the RAS inhibitor binds the RAS in its “on” position.
Embodiment I-101. The method of Embodiment I-100, wherein the RAS inhibitor is a KRAS(ON) inhibitor.
Embodiment I-102. The method of Embodiment I-101, wherein the KRAS(ON) inhibitor is a KRAS^{G12C}(ON) inhibitor.
Embodiment I-103. The method of any one of Embodiments I-80 to I-85, Embodiment I-88 or Embodiments I-90 to I-102, wherein the RAS inhibitor is selected from compounds A1-A741 of Appendix B-1, or a pharmaceutically acceptable salt thereof.
Embodiment I-104. The method of any one of Embodiments I-80 to I-85, Embodiment I-88 or Embodiments I-90 to I-102, wherein the RAS inhibitor is a compound, or a pharmaceutically acceptable salt thereof, of Appendix B-1, Formula VIb,

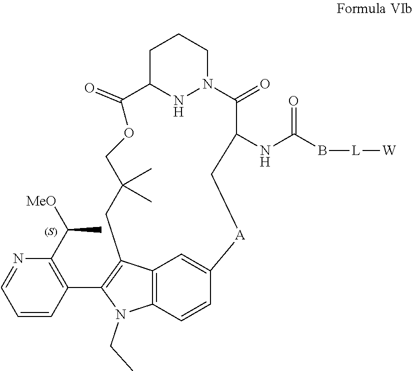

wherein A is a 3 to 6-membered heterocycloalkylene, a phenylene, or a hydroxy-substituted phenylene; B is —CH(C₁-C₆ alkyl)-; L is a linker selected from the following:

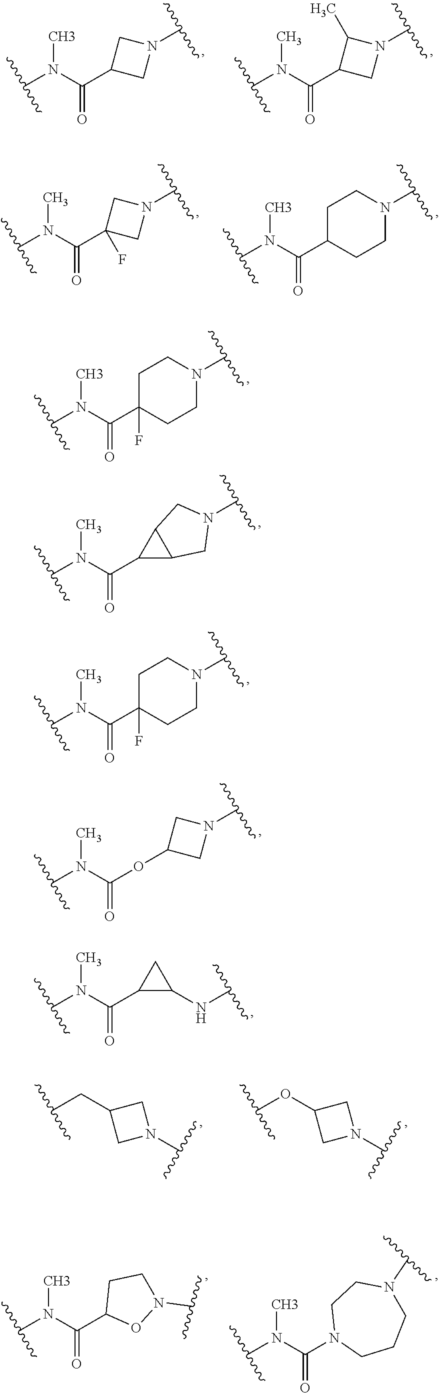

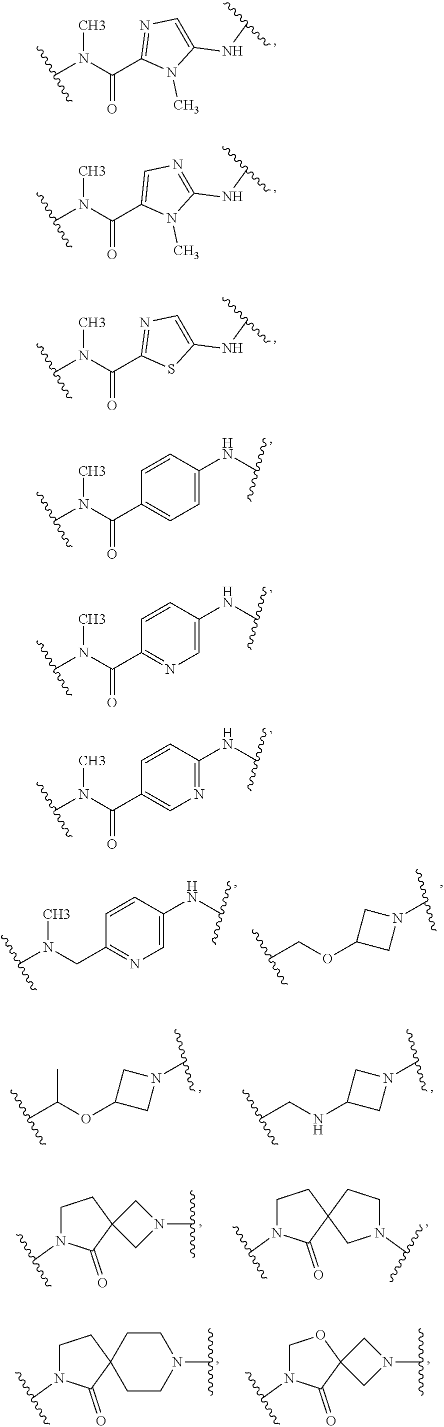

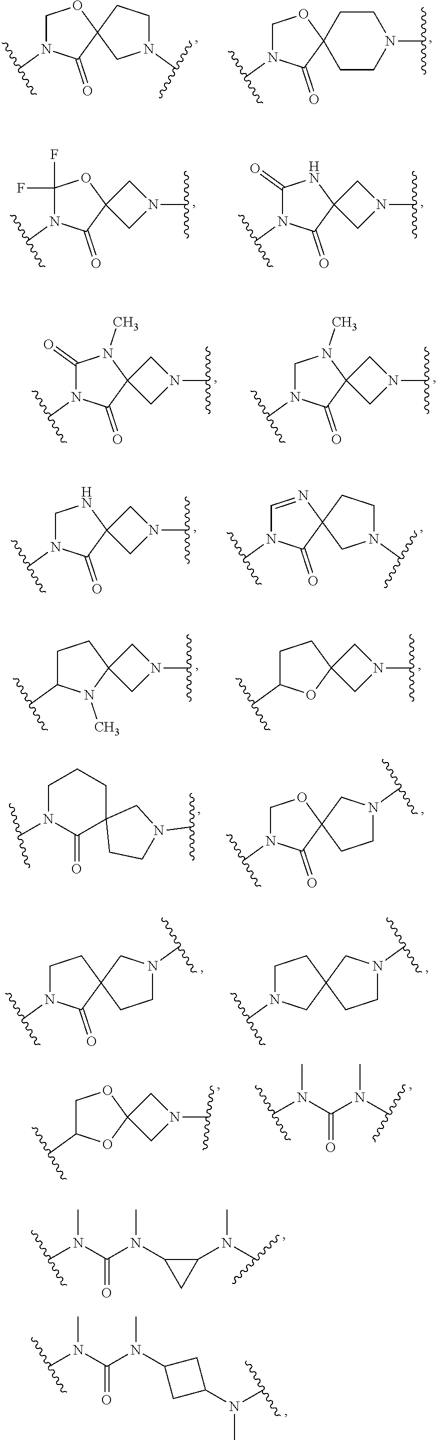

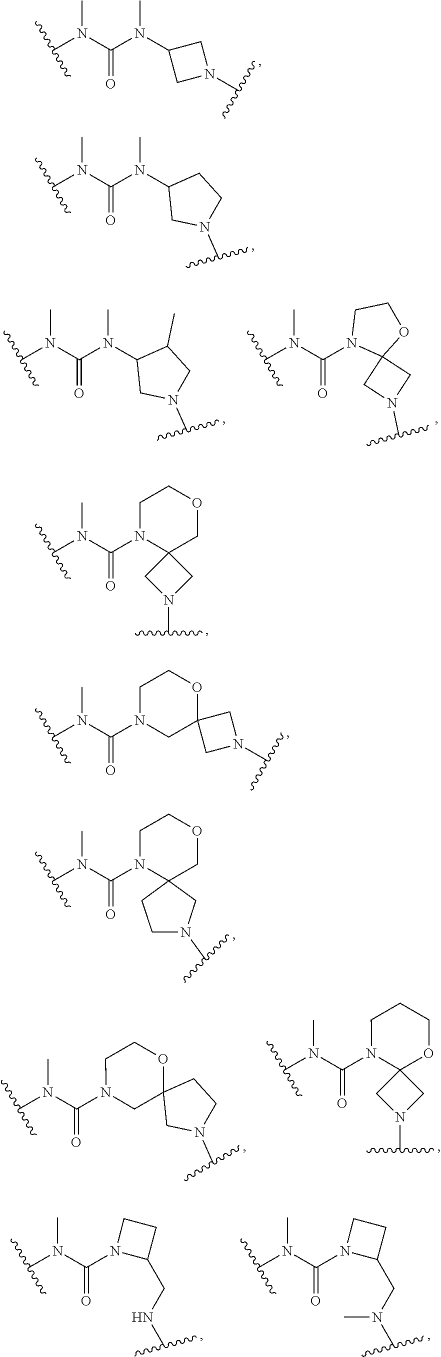

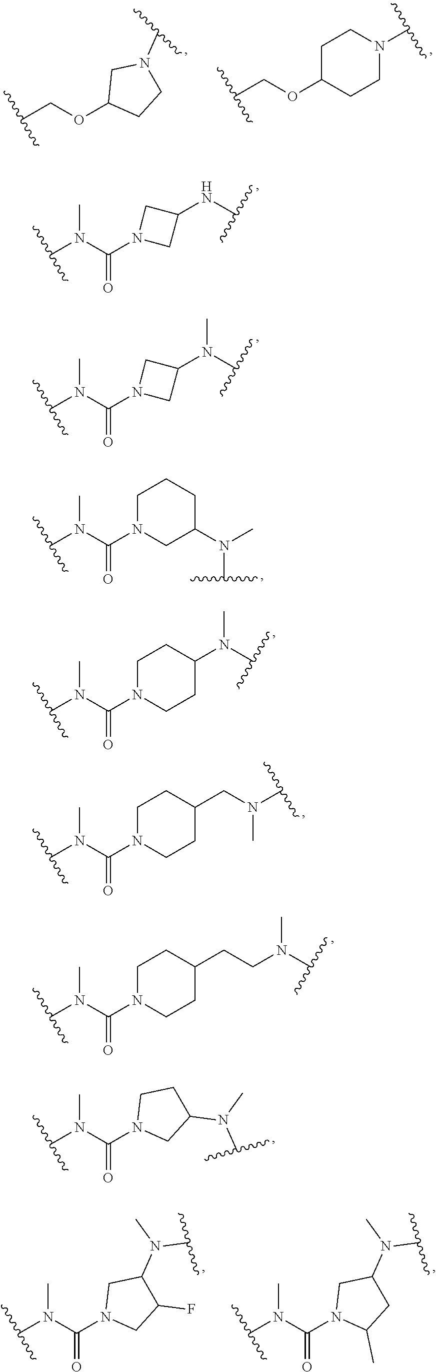

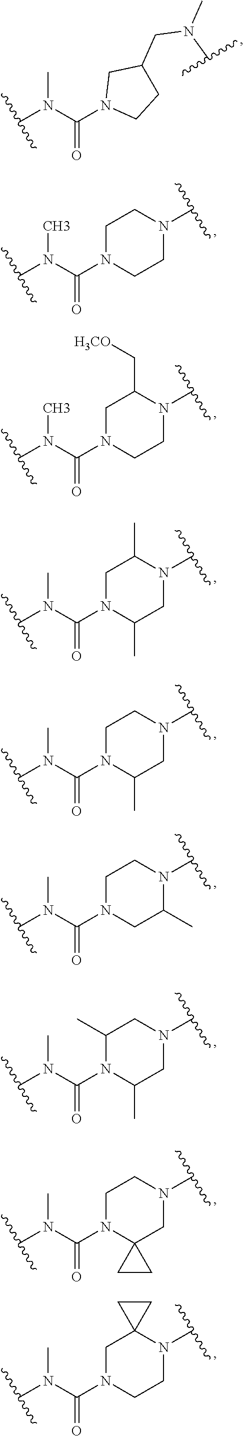

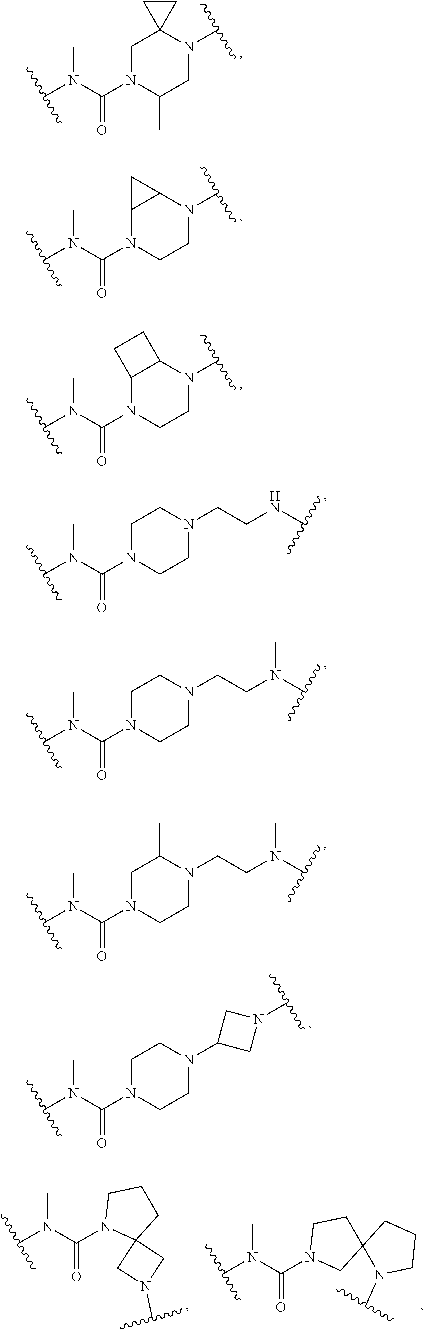

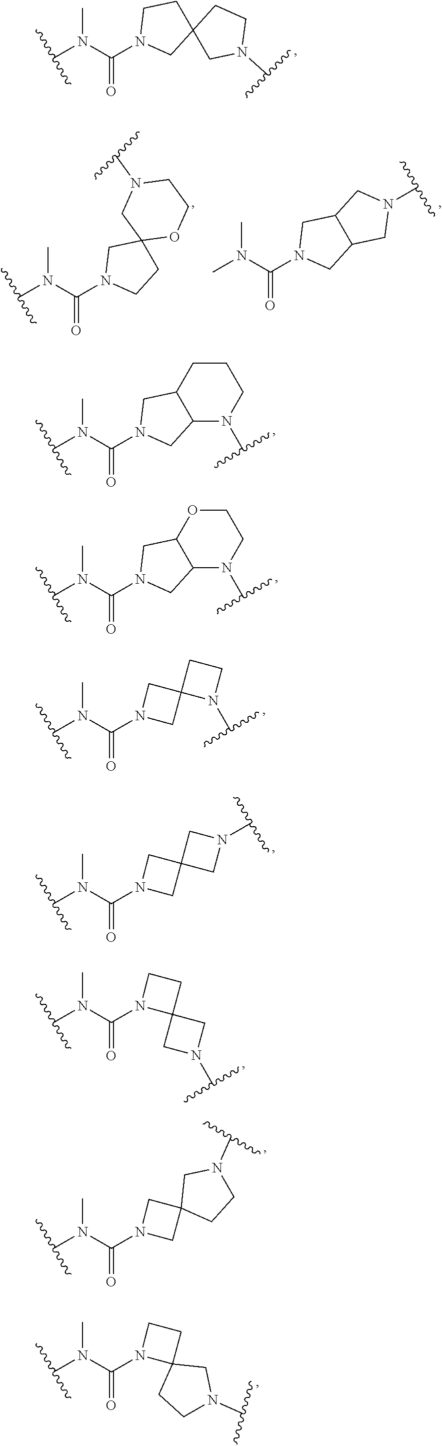

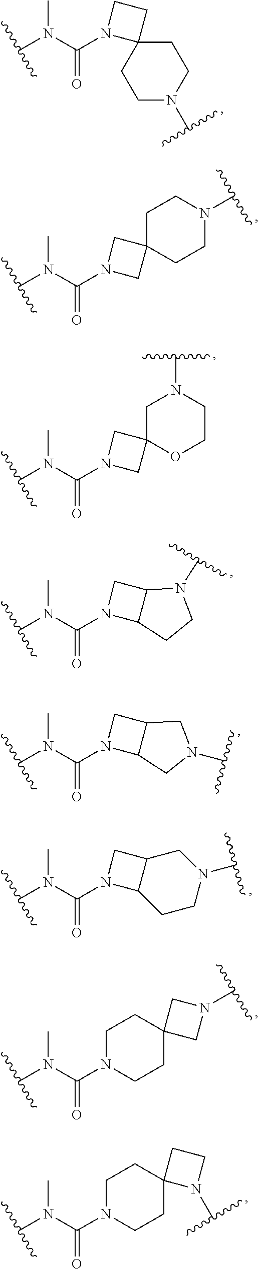

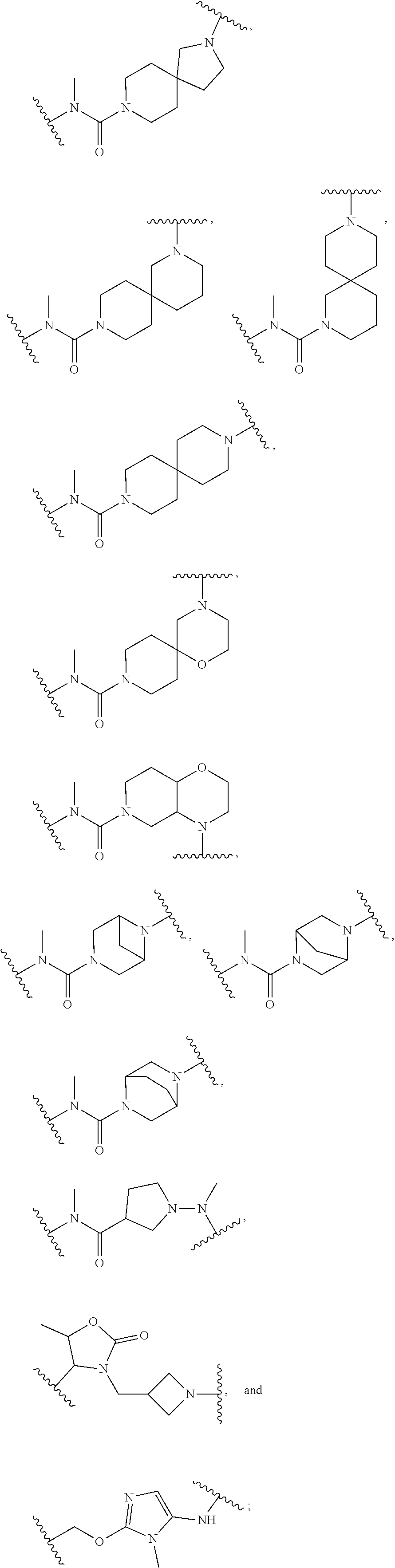

and
W is a cross-linking group selected from the following:

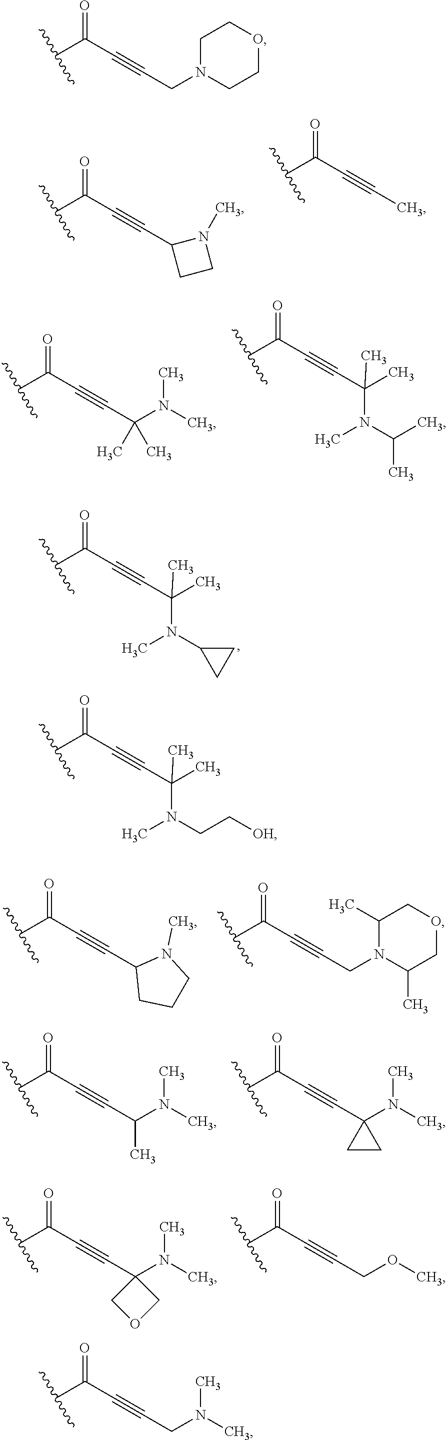

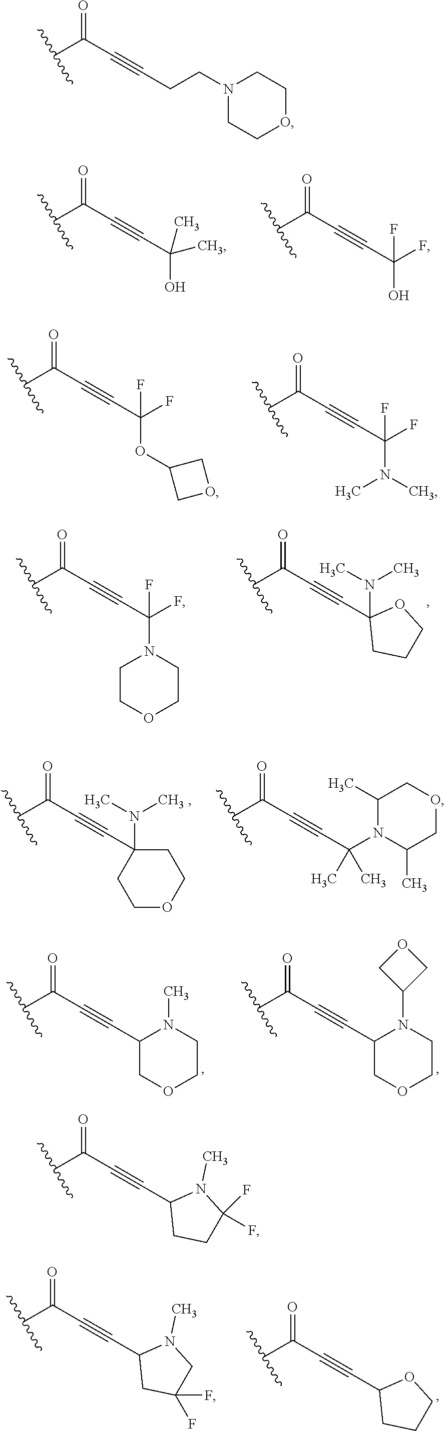

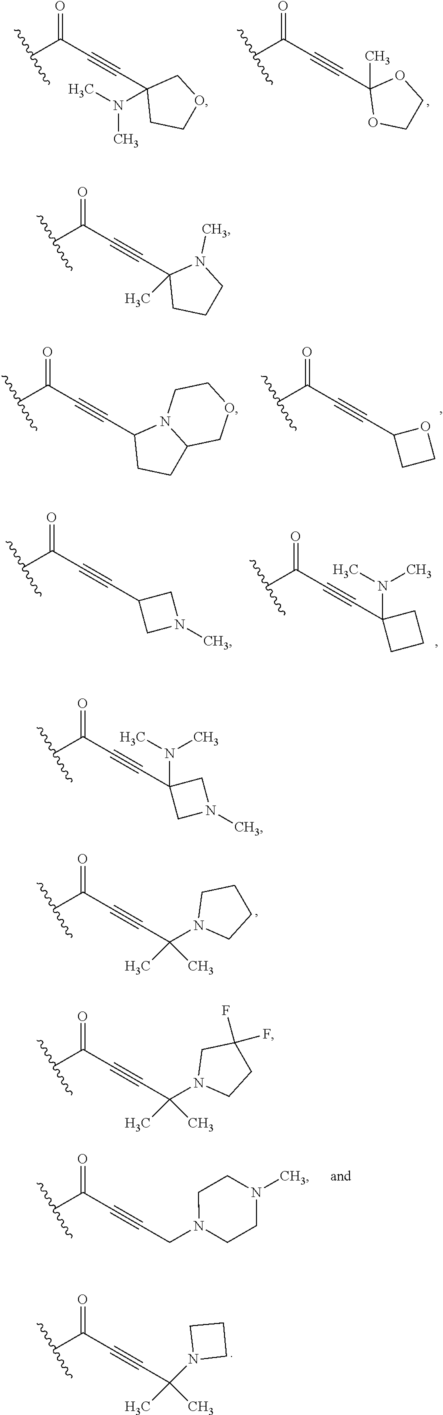

Embodiment I-105. The method of any one of Embodiments I-80 to I-85, Embodiment I-88 or Embodiments I-90 to I-104, wherein the RAS inhibitor is selected from compound A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof.
Embodiment I-106. The method of any one of Embodiments I-80 to I-85, Embodiment I-88 or Embodiments I-90 to I-105, wherein the RAS inhibitor is Compound A, or a pharmaceutically acceptable salt thereof.
Embodiment I-107. The method of any one of Embodiments I-80 to I-85, Embodiment I-88 or Embodiments I-90 to I-107, wherein the RAS inhibitor is Compound B, or a pharmaceutically acceptable salt thereof.
Embodiment I-108. The method of any one of Embodiments I-80 to I-107, wherein the tumor is caused by a cancer.
Embodiment I-109. The method of any one of Embodiments I-80 to I-83, Embodiments I-86 to I-87, or Embodiments I-89 to I-107, wherein the cancer is a RAS G12C cancer.
Embodiment I-110. The method of any one of Embodiments I-80 to I-109, wherein the cancer comprises a KRAS^{G12C} mutation.
Embodiment I-111. The method of any one of Embodiments I-80 to I-110, wherein the cancer comprises co-occurring KRAS^{G12C} and STK11 mutations.
Embodiment I-112. The method of any one of Embodiments I-80 to I-110, wherein the cancer is a Non-Small Cell Lung Cancer (NSCLC).
Embodiment I-113. The method of any one of Embodiments I-80 to I-111, wherein the cancer is a colorectal cancer.
Embodiment I-114. The method of any one of Embodiments I-80 to I-113, wherein the cancer is selected from pancreatic cancer, colorectal cancer, non-small cell lung cancer, squamous cell lung carcinoma, thyroid gland adenocarcinoma, and a hematological cancer.
Embodiment I-115. The method of any one of Embodiments I-80 to I-114, wherein the cancer comprises co-occurring KRAS^{G12C} and PIK3CA^{E545K} mutations.
Embodiment I-116. The method of any one of Embodiments I-80 to I-111 or Embodiments I-113 to I-115, wherein the cancer is a colorectal cancer.
Embodiment I-117. The method of any one of Embodiments I-80 to I-116, wherein the method results in tumor regression.
Embodiment I-118. The method of any one of Embodiments I-1 to I-117, wherein the method results in an improved lifespan for the subject as compared to the lifespan of a similar subject that has not received a treatment with the RAS inhibitor and the bi-steric mTOR inhibitor.

Embodiment II-1. A method for delaying or preventing acquired resistance to AMG 510 or MRTX849, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the subject has already received or will receive administration of AMG 510 or MRTX849, or a pharmaceutically acceptable salt thereof, wherein the effective amount is an amount effective to delay or prevent acquired resistance to AMG 510 or MRTX849, or a pharmaceutically acceptable salt thereof, in the subject.

Embodiment II-2. A method for delaying or preventing acquired resistance to a compound of Formula IVb of Appendix B-1, or a pharmaceutically acceptable salt thereof:

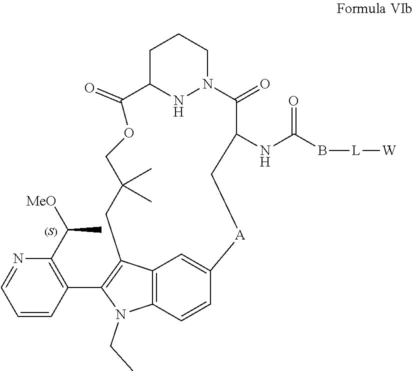

wherein A is a 3 to 6-membered heterocycloalkylene, a phenylene, or a hydroxy-substituted phenylene; B is —CH(C₁-C₆ alkyl)-; L is a linker selected from the following:

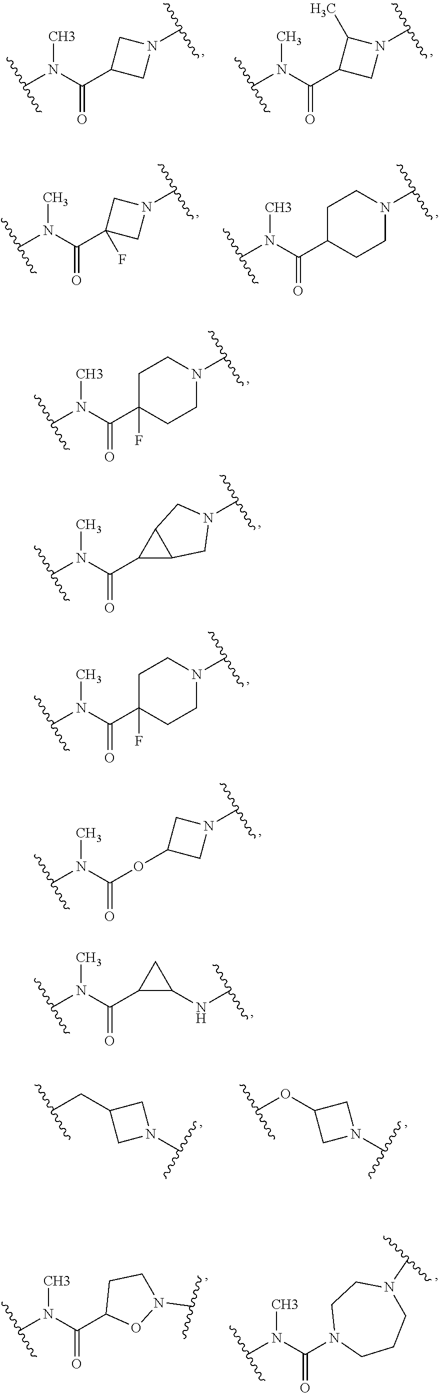

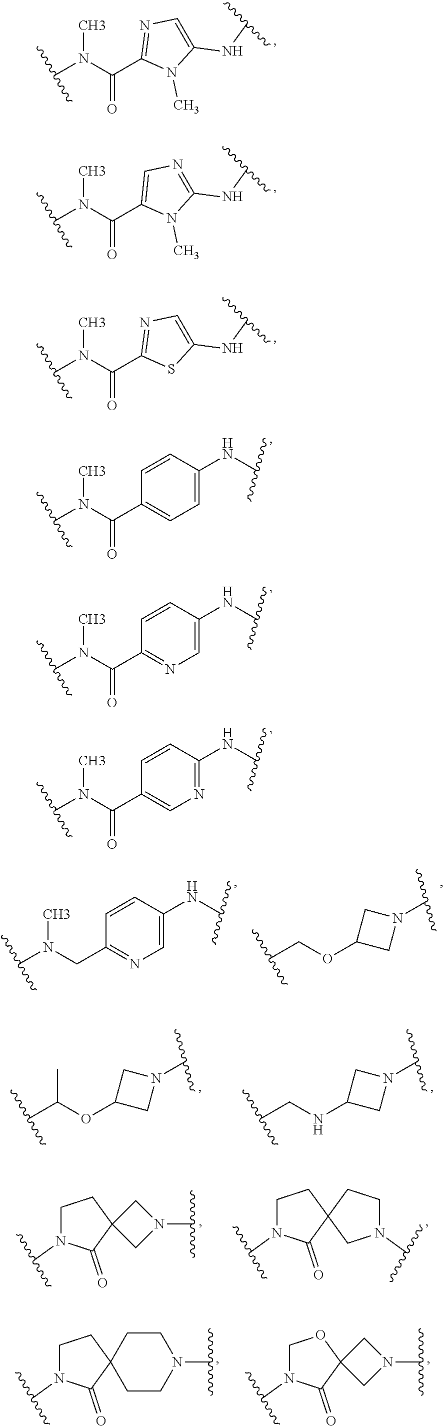

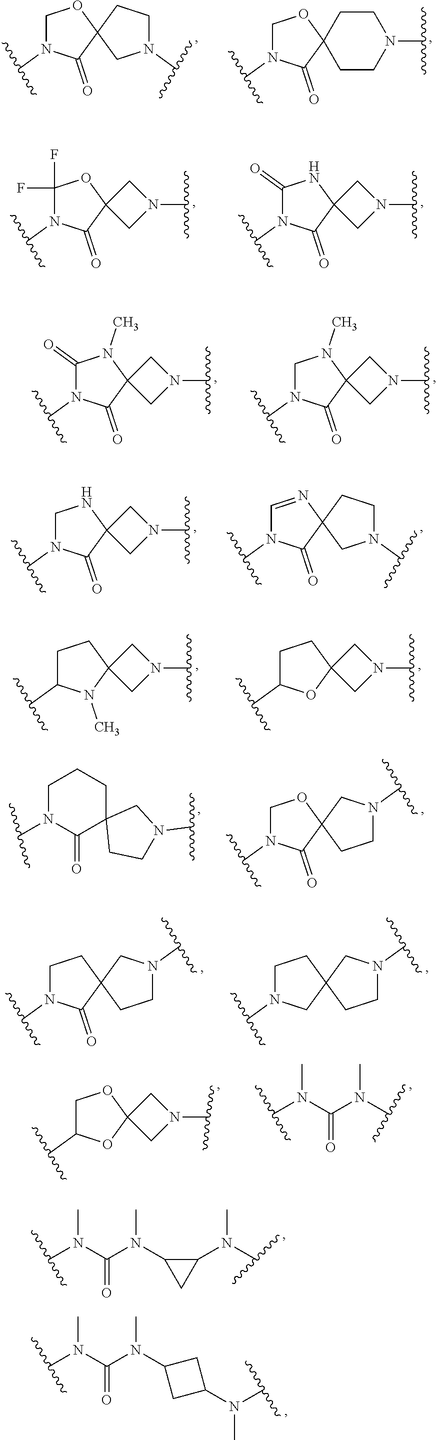

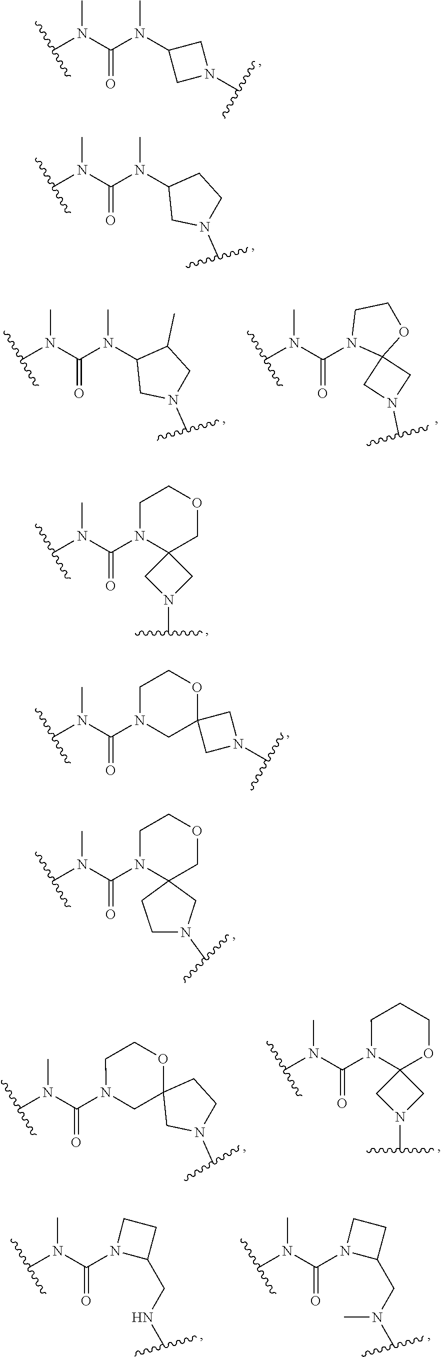

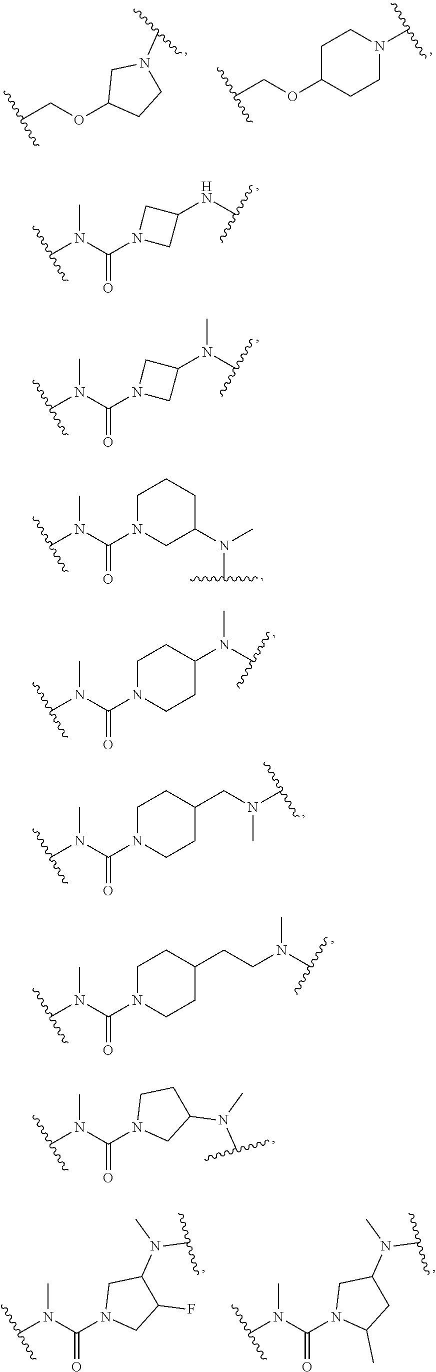

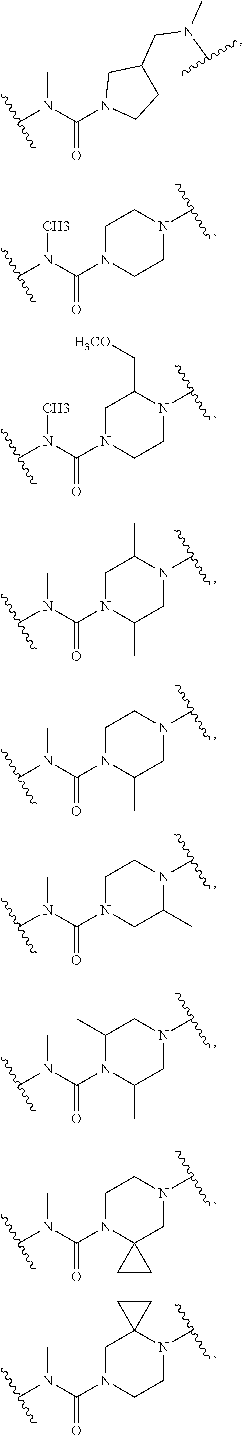

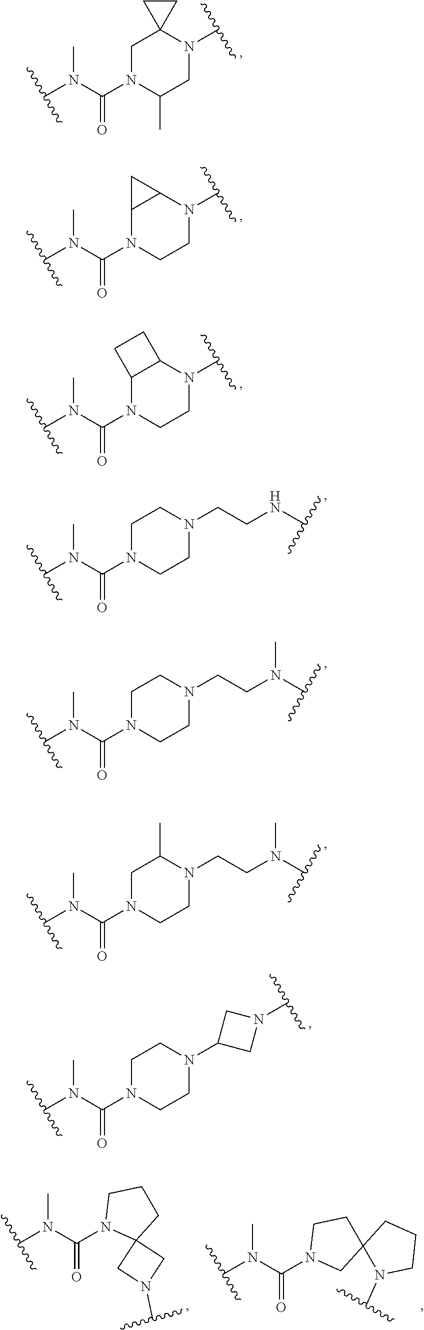

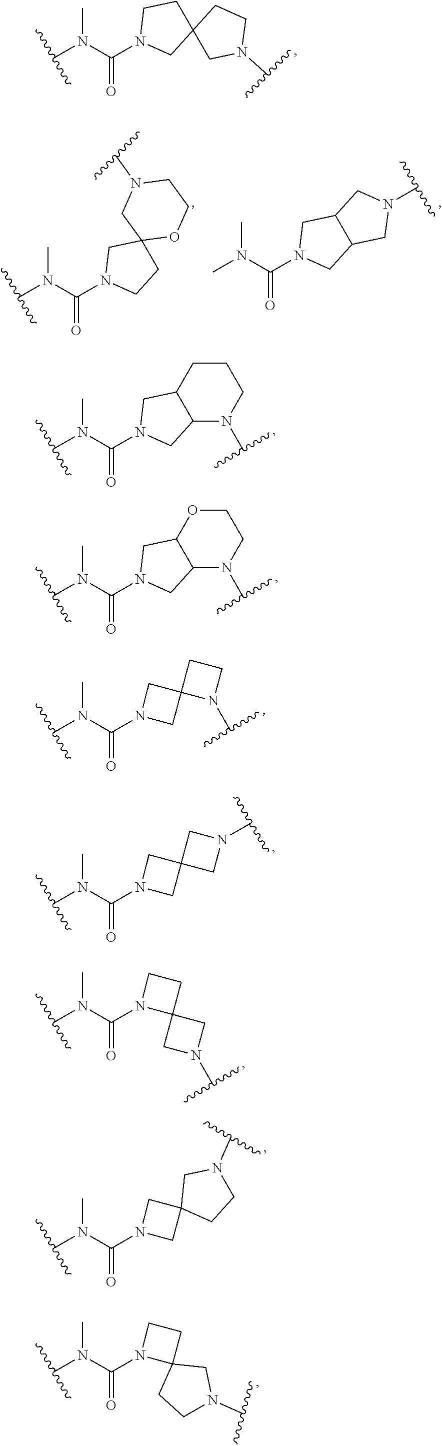

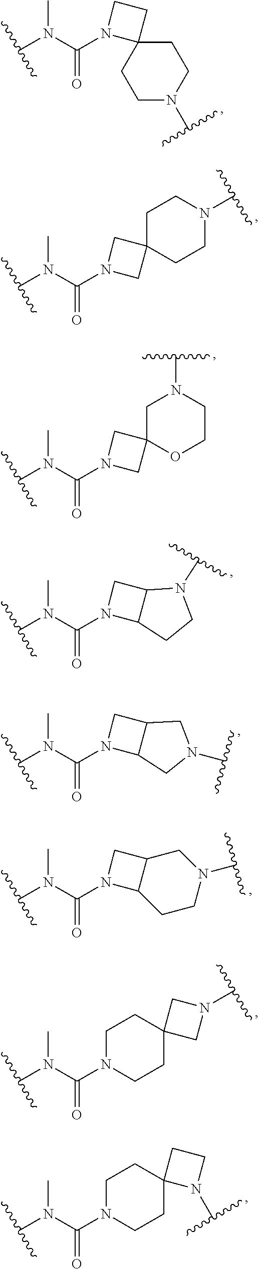

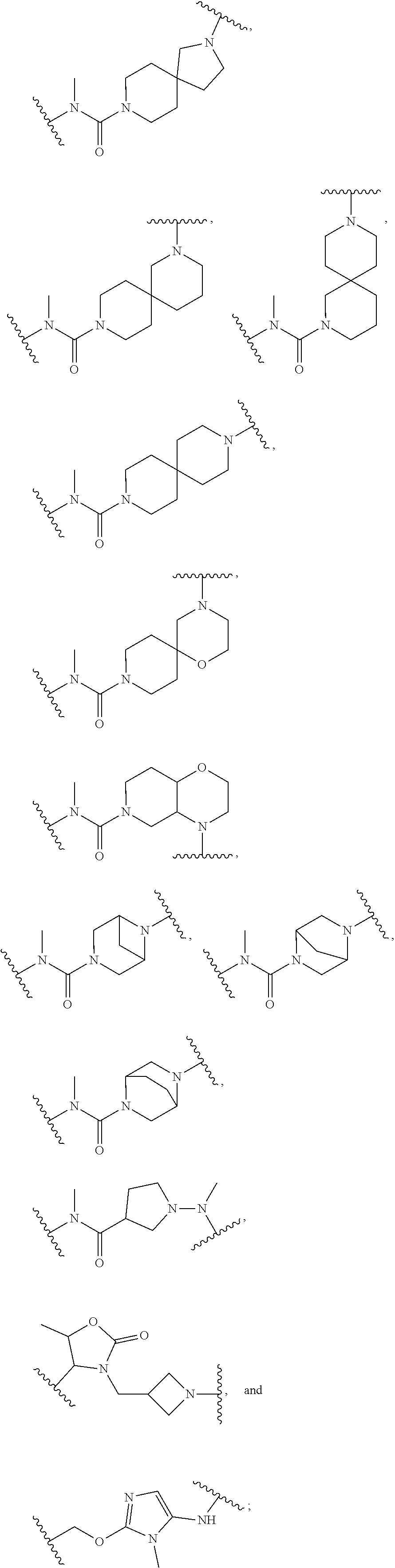

and
W is a cross-linking group selected from the following:

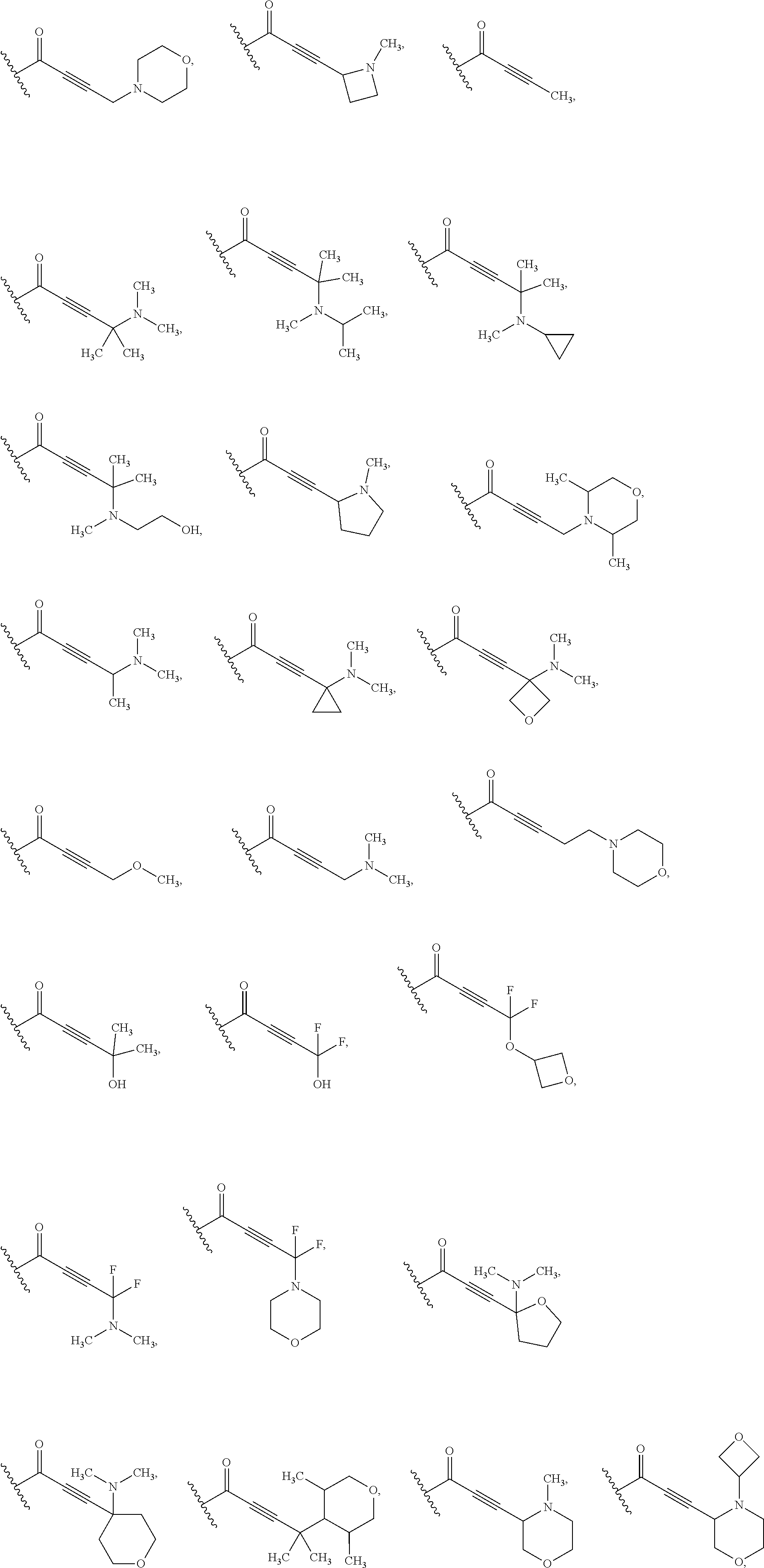

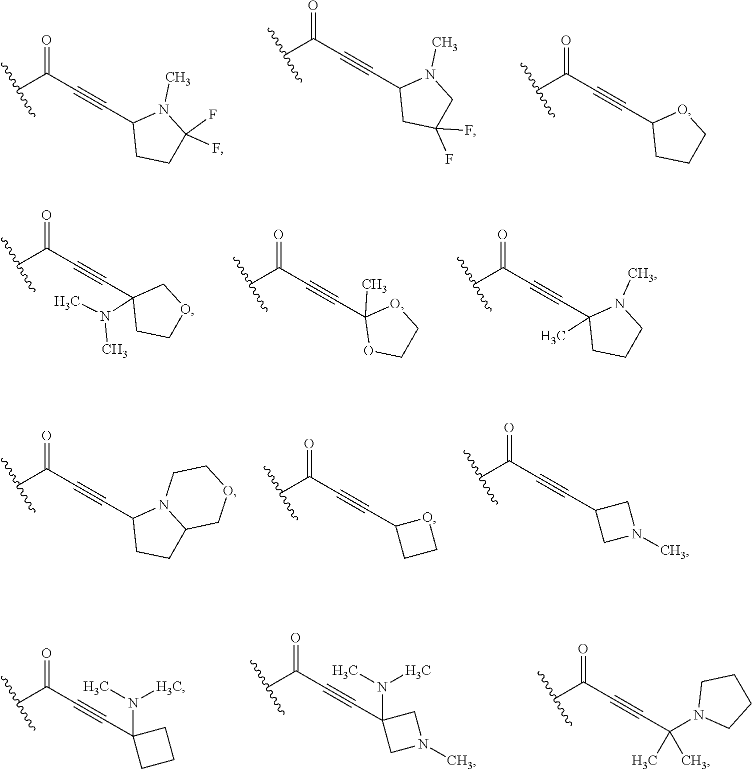

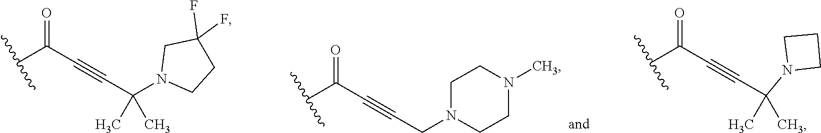 [SMILES: *C(=O)C#CC(C)(C)N1CCC(F)(F)C1 ; *C(=O)C#CCN1CCN(C)CC1 ; *C(=O)C#CC(C)(C)N1CCC1]

in a subject having a RAS^{G12C} mutated NSCLC or colorectal cancer, comprising administering to the subject an effective amount of RMC-5552, or a stereoisomer or tautomer thereof, wherein the subject has already received or will receive administration of the compound, or a pharmaceutically acceptable salt thereof, wherein the effective amount is an amount effective to delay or prevent acquired resistance to the compound, or a pharmaceutically acceptable salt thereof, in the subject.
Embodiment II-3. A method for delaying or preventing acquired resistance to a compound selected from compound A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the subject has already received or will receive administration of the compound, or a pharmaceutically acceptable salt thereof, wherein the effective amount is an amount effective to delay or prevent acquired resistance to the compound, or a pharmaceutically acceptable salt thereof, in the subject.
Embodiment II-4. A method for delaying or preventing acquired resistance to Compound A, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the subject has already received or will receive administration of Compound A, or a pharmaceutically acceptable salt thereof, wherein the effective amount is an amount effective to delay or prevent acquired resistance to Compound B, or a pharmaceutically acceptable salt thereof, in the subject.
Embodiment II-5. A method for delaying or preventing acquired resistance to Compound B, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the subject has already received or will receive administration of Compound B, or a pharmaceutically acceptable salt thereof, wherein the effective amount is an amount effective to delay or prevent acquired resistance to Compound B, or a pharmaceutically acceptable salt thereof, in the subject.
Embodiment III-1. A method of treating acquired resistance to AMG 510 or MRTX849, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the effective amount is an amount effective to treat acquired resistance to AMG 510 or MRTX849, or a pharmaceutically acceptable salt thereof, in the subject.
Embodiment III-2. A method of treating acquired resistance to a compound of Formula IVb of Appendix B-1, or a pharmaceutically acceptable salt thereof:

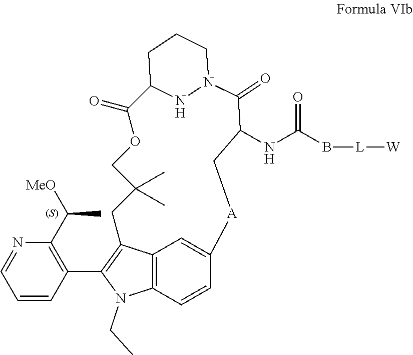

wherein A is a 3 to 6-membered heterocycloalkylene, a phenylene, or a hydroxy-substituted phenylene; B is —CH(C₁-C₆ alkyl)-; L is a linker selected from the following:

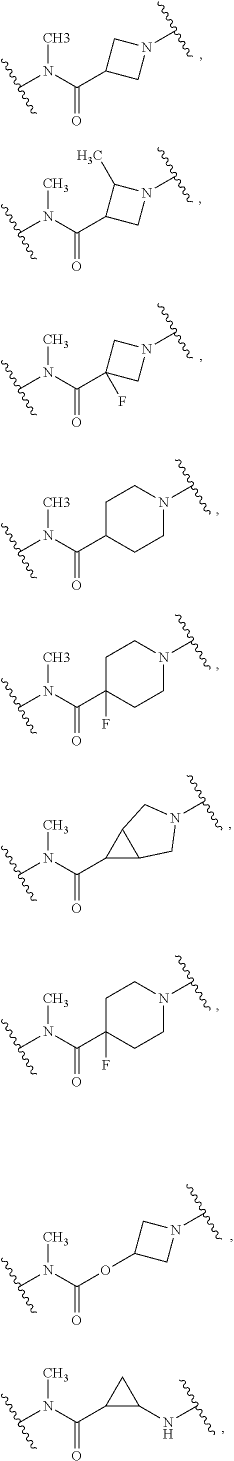

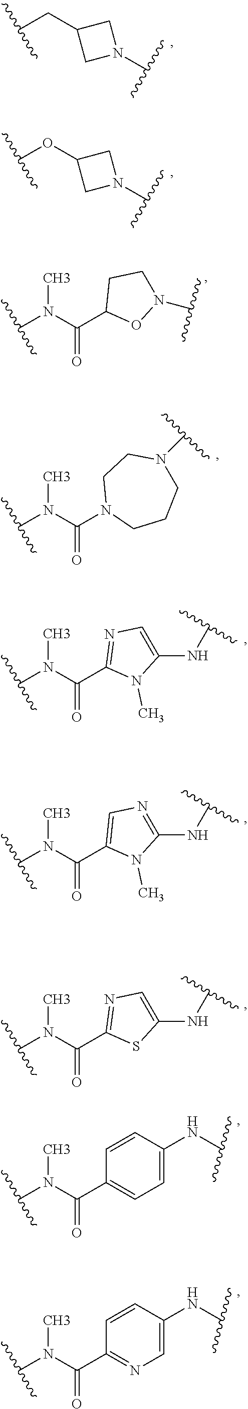

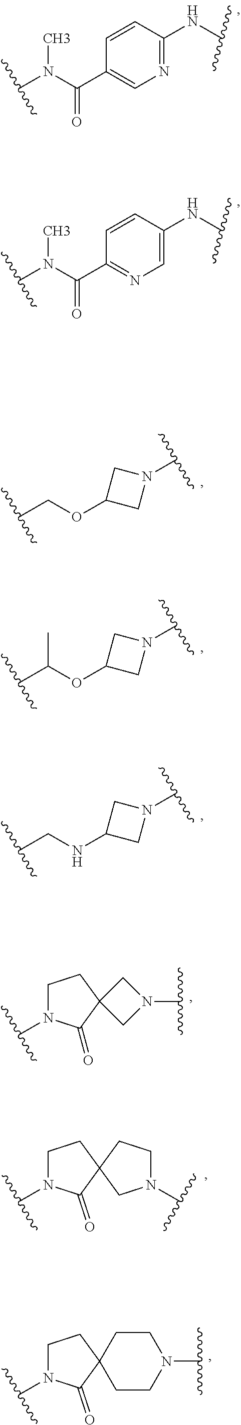

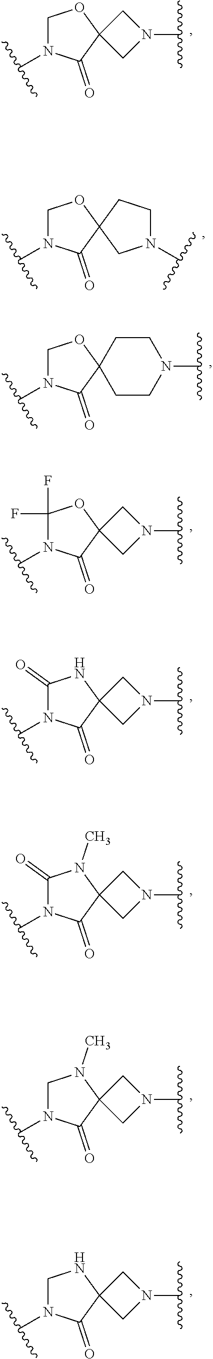

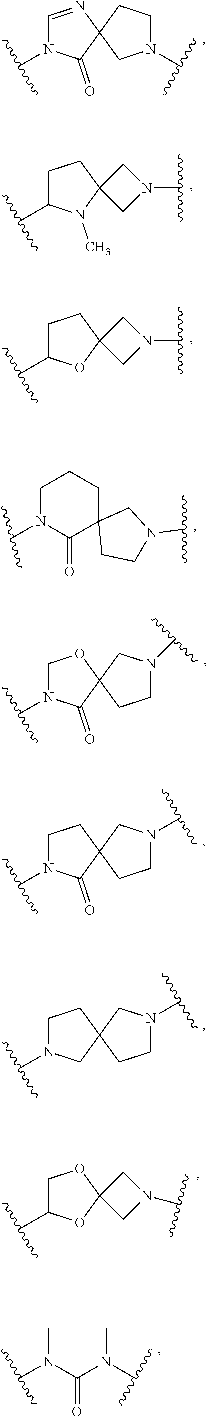

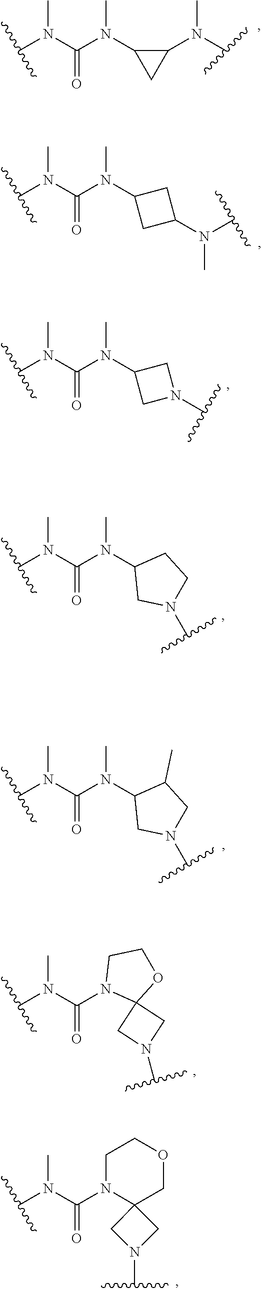

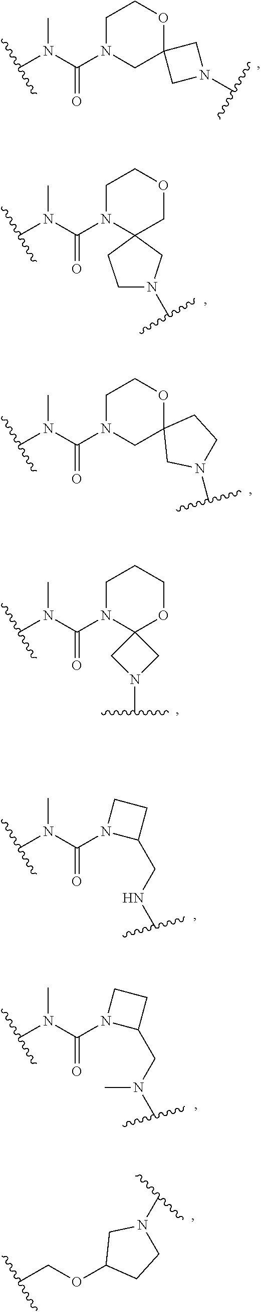

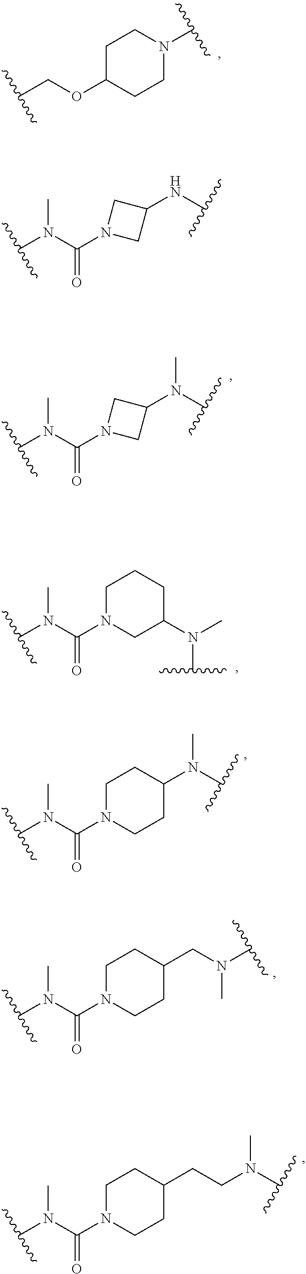

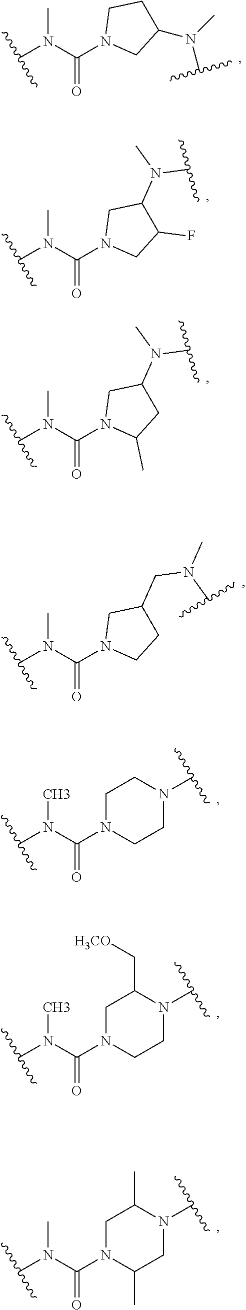

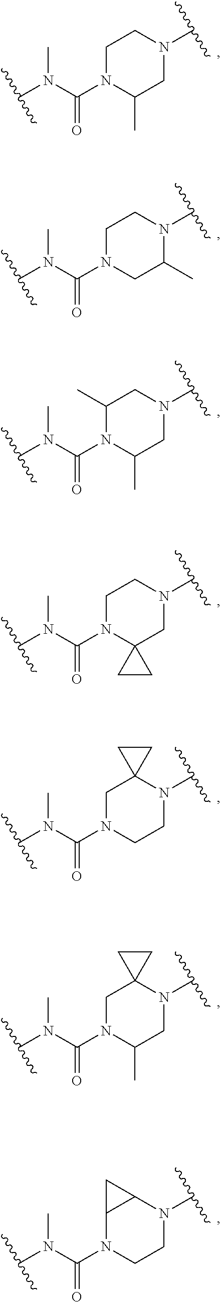

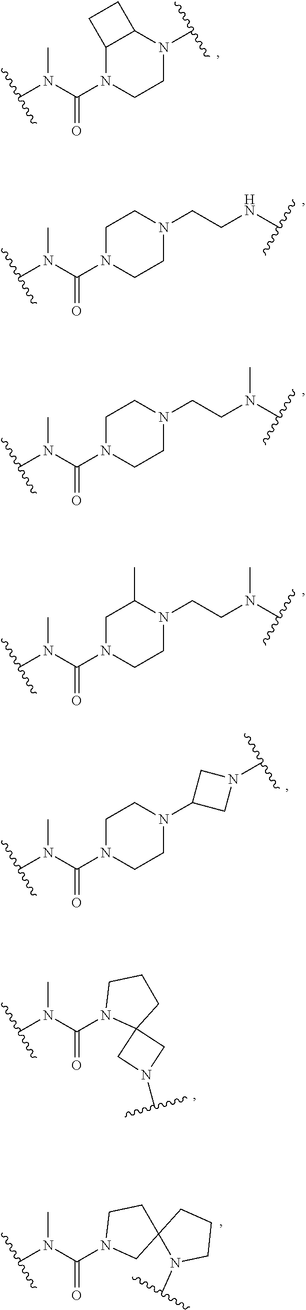

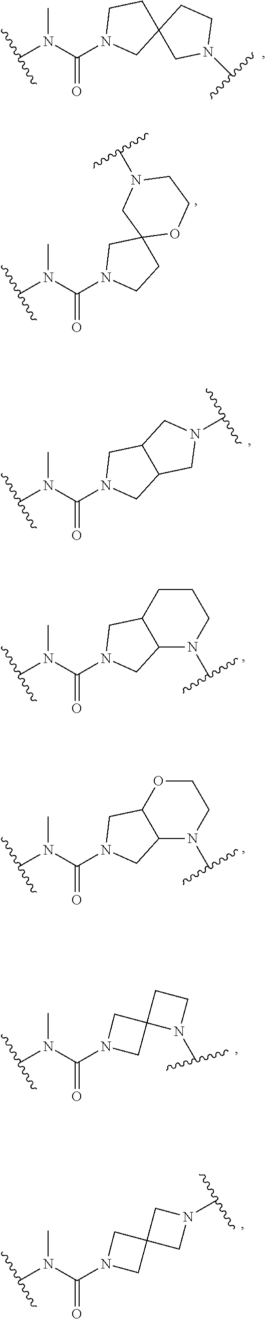

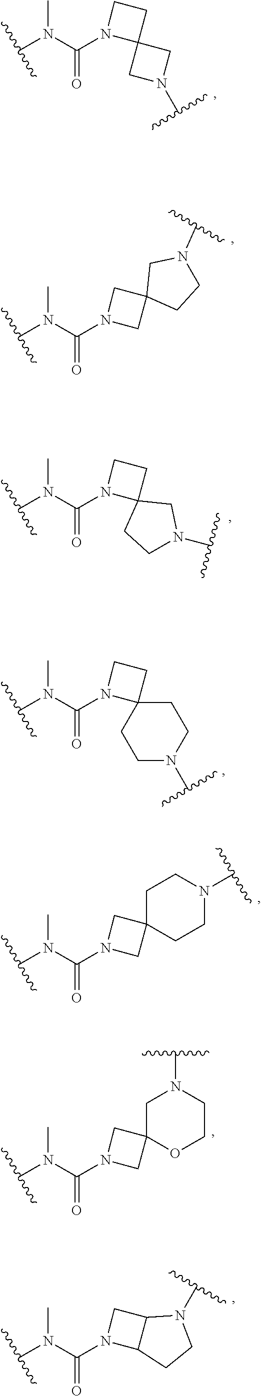

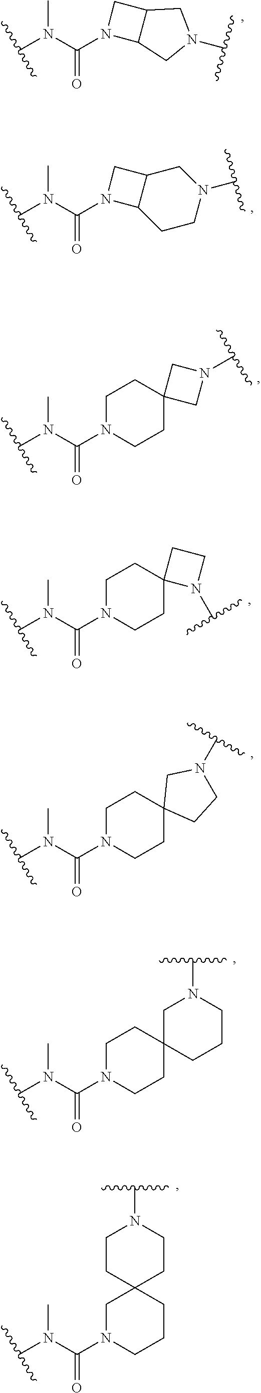

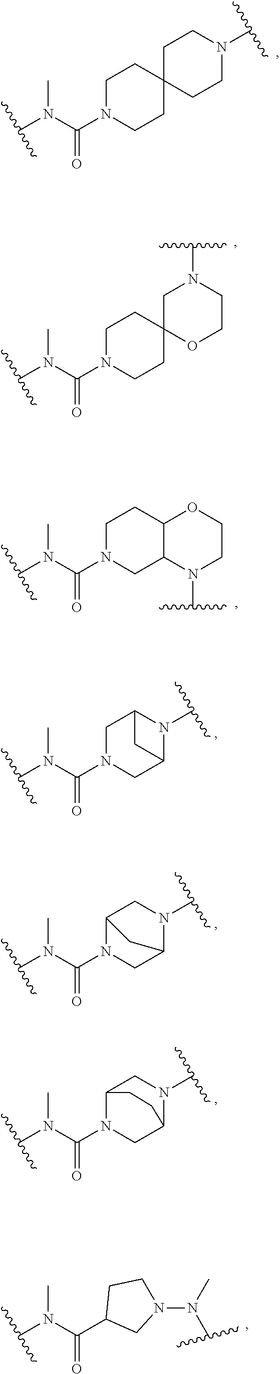

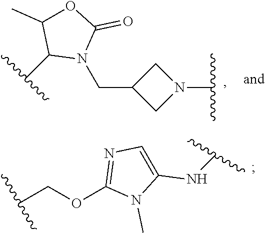

and
W is a cross-linking group selected from the following:

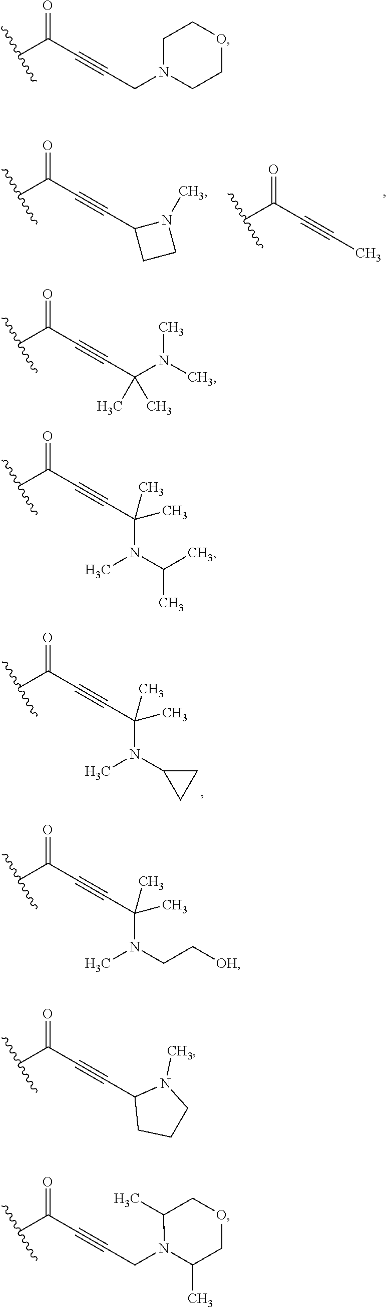

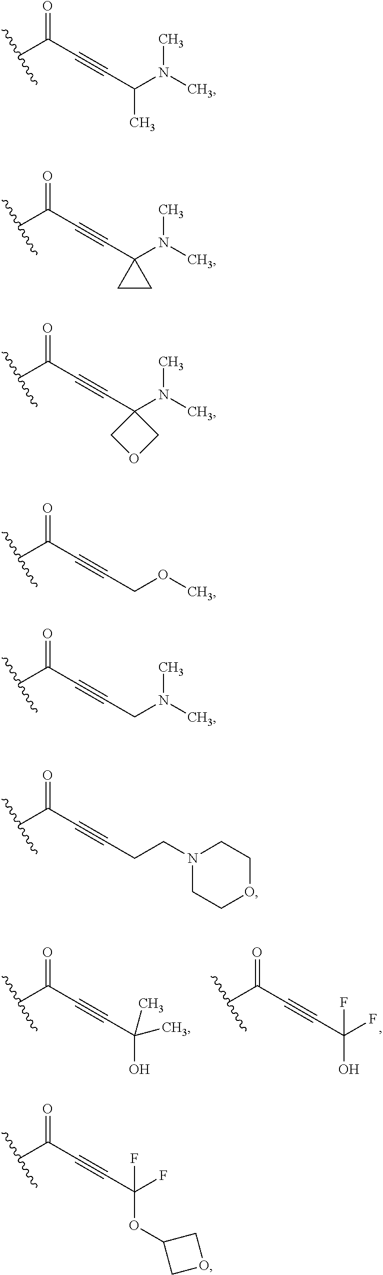

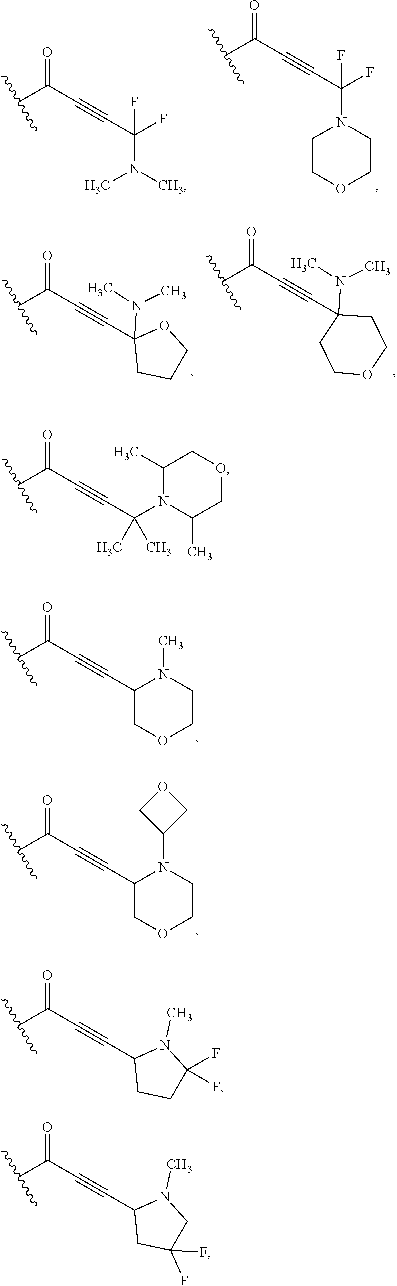

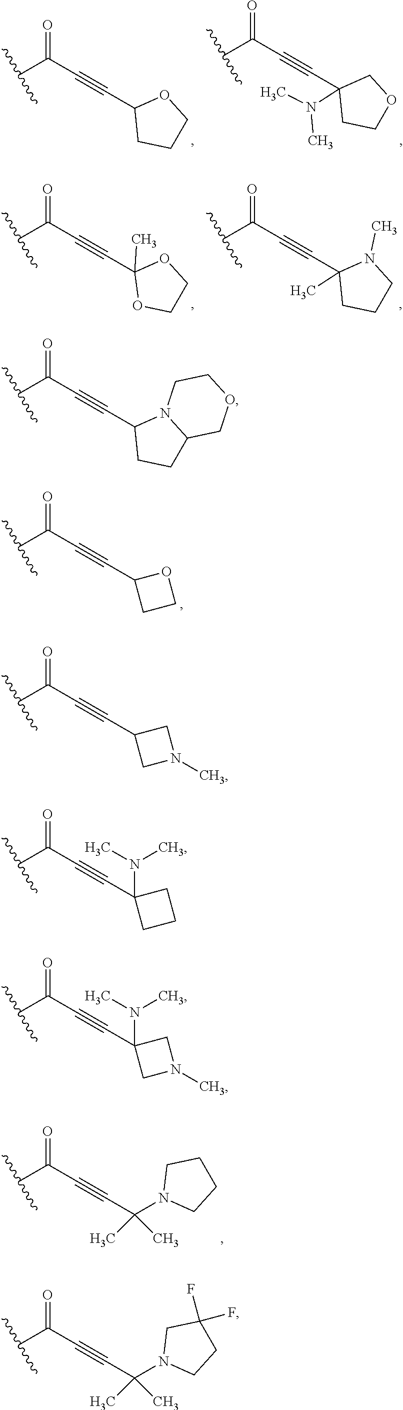

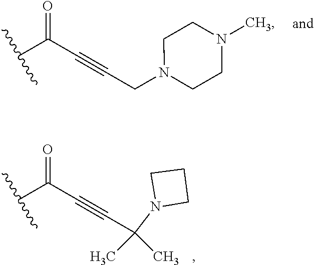 [SMILES: *C(=O)C#CCN1CCN(C)CC1 ; *C(=O)C#CC(C)(C)N1CCC1]

in a subject having a RAS^{G12C} mutated NSCLC or colorectal cancer, comprising administering to the subject an effective amount of RMC-5552, or a stereoisomer or tautomer thereof, wherein the effective amount is an amount effective to treat acquired resistance to the compound, or a pharmaceutically acceptable salt thereof, in the subject.
Embodiment III-3. A method of treating acquired resistance to a compound selected from compound A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the effective amount is an amount effective to treat acquired resistance to the compound, or a pharmaceutically acceptable salt thereof, in the subject.
Embodiment III-4. A method of treating acquired resistance to Compound A, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the effective amount is an amount effective to treat acquired resistance to Compound A, or a pharmaceutically acceptable salt thereof, in the subject.
Embodiment III-5. A method of treating acquired resistance to Compound B, or a pharmaceutically acceptable salt thereof, in a subject having a RAS^{G12C} mutated NSCLC or colorectal cancer, comprising administering to the subject an effective amount of RMC-5552, or a stereoisomer or tautomer thereof, wherein the effective amount is an amount effective to treat acquired resistance to Compound B, or a pharmaceutically acceptable salt thereof, in the subject.
Embodiment IV-1. A method of treating a subject having a RAS^{G12C} mutated NSCLC or colorectal cancer, comprising administering to the subject an effective amount of RMC-5552, or a stereoisomer or tautomer thereof, in combination with AMG 510 or MRTX849, or a pharmaceutically acceptable salt thereof.
Embodiment IV-1. A method of treating a subject having a RAS^{G12C} mutated NSCLC or colorectal cancer, comprising administering to the subject an effective amount of RMC-5552, or a stereoisomer or tautomer thereof, in combination with a compound of Formula IVb of Appendix B-1, or a pharmaceutically acceptable salt thereof:

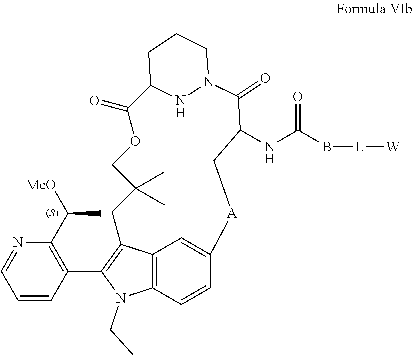

wherein A is a 3 to 6-membered heterocycloalkylene, a phenylene, or a hydroxy-substituted phenylene; B is —CH(C₁-C₆ alkyl)-; L is a linker selected from the following:

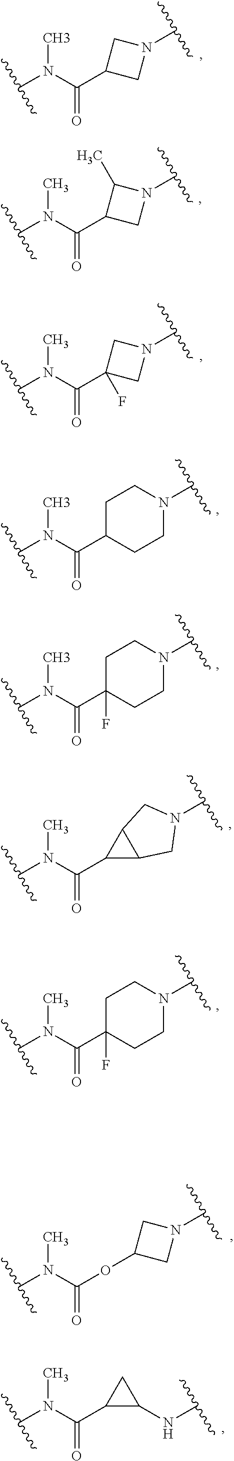

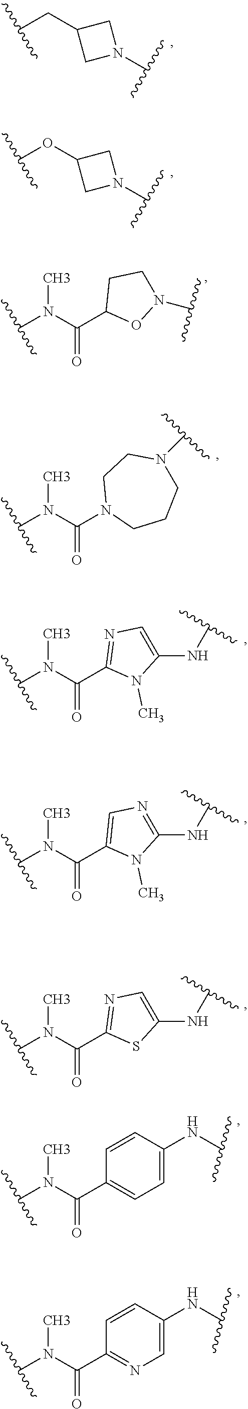

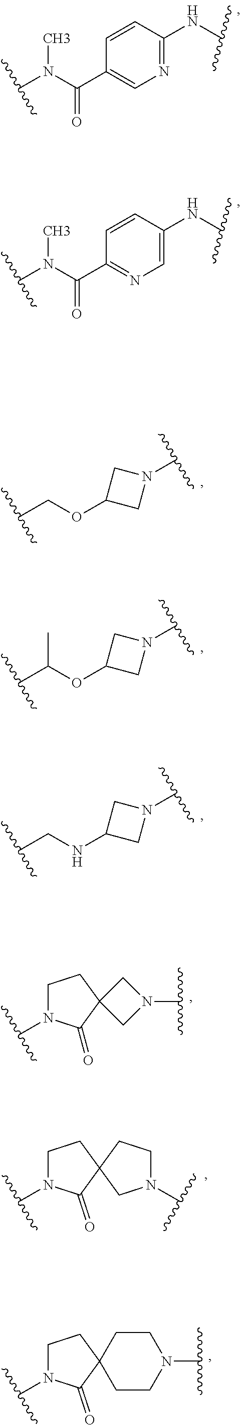

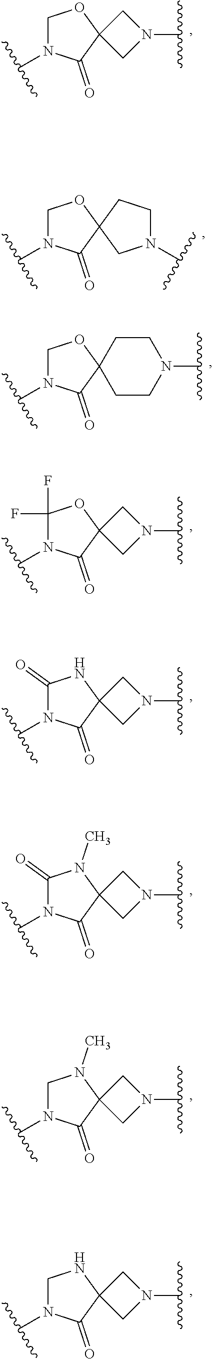

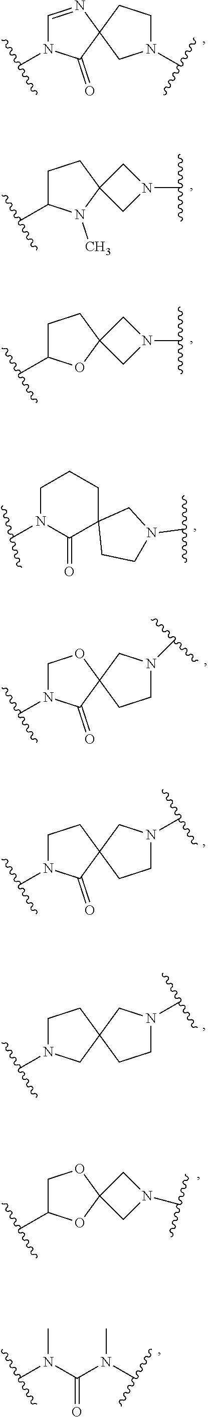

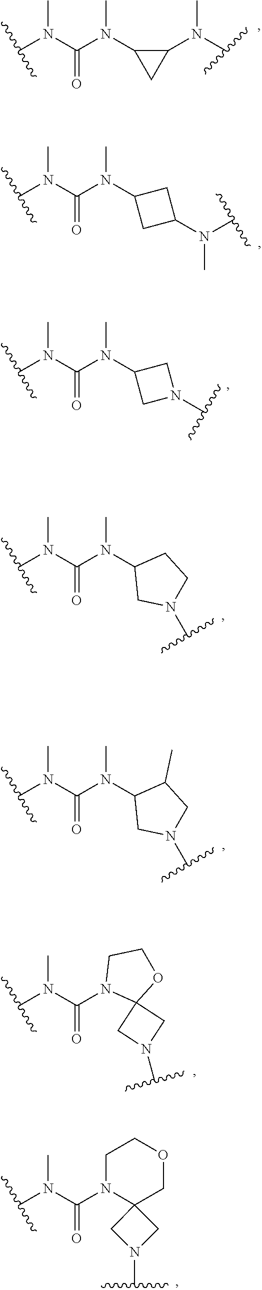

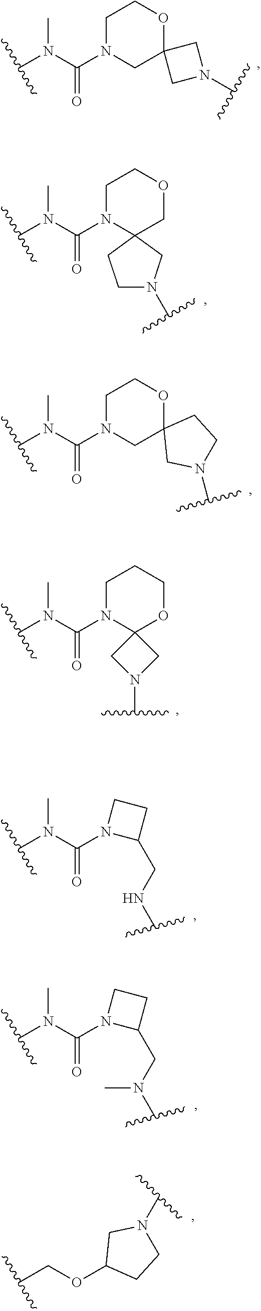

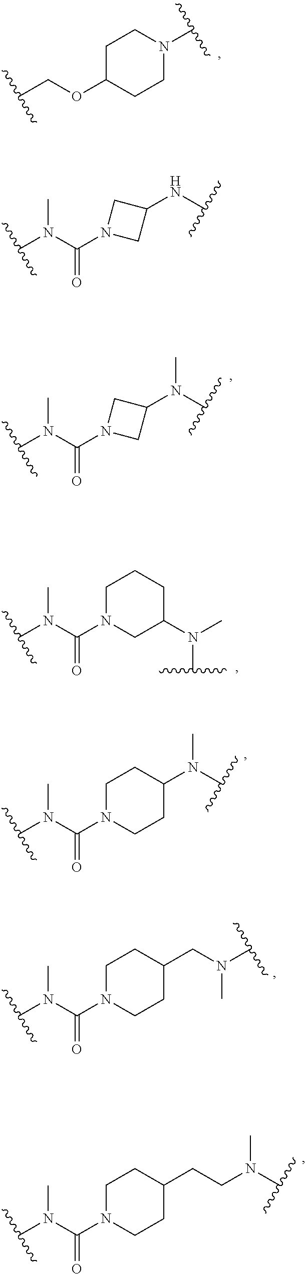

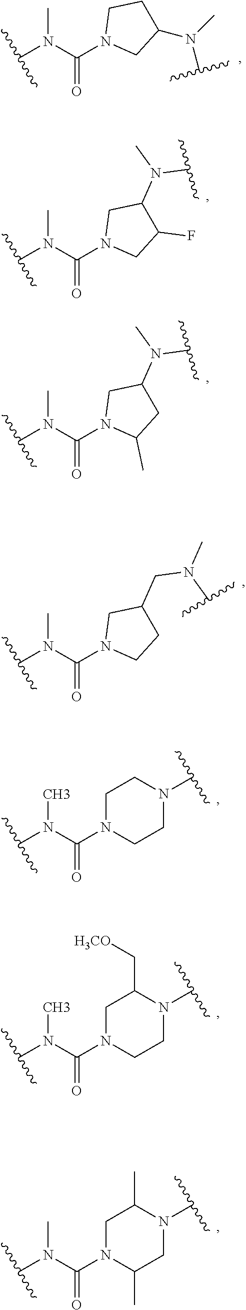

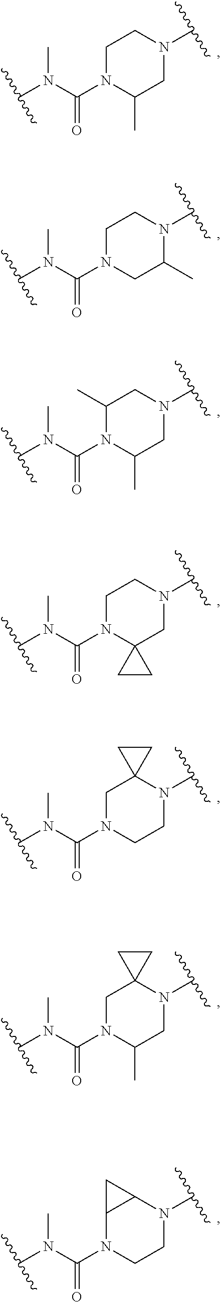

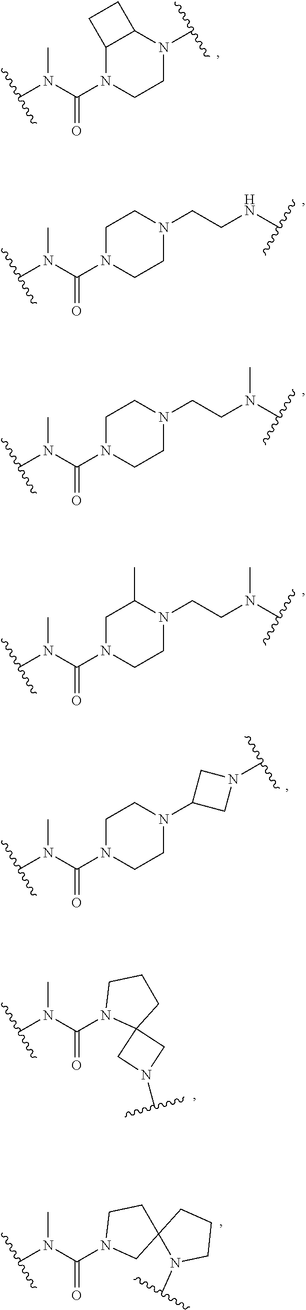

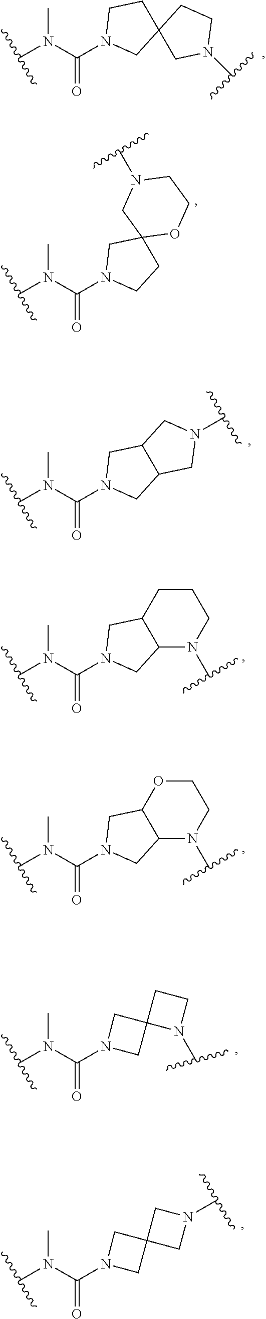

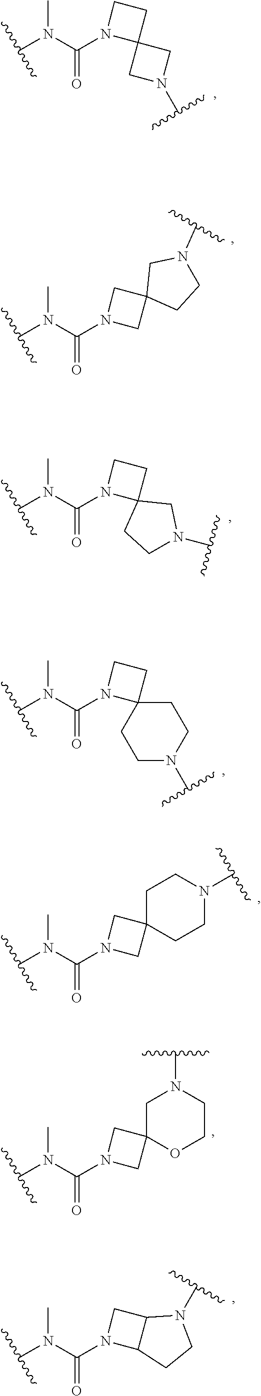

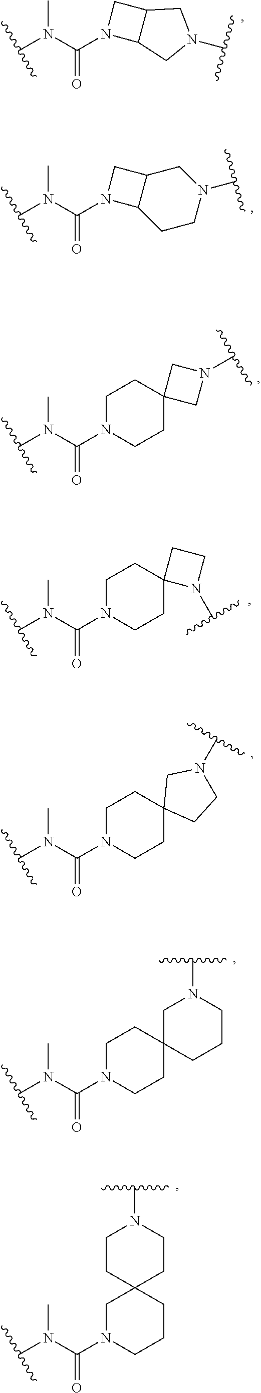

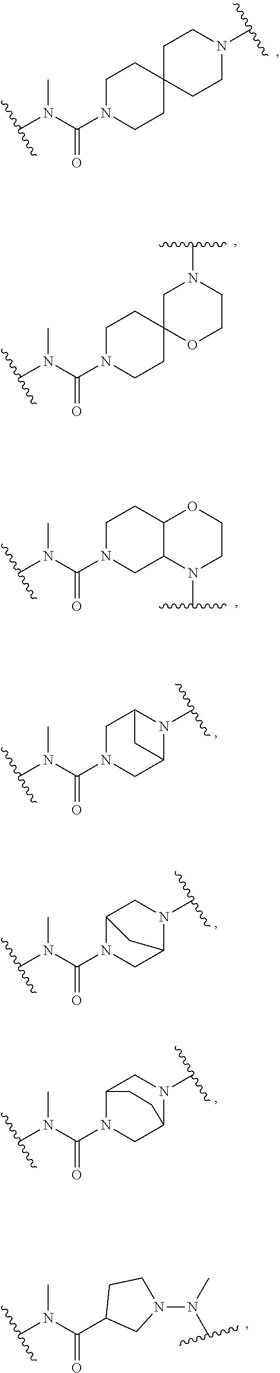

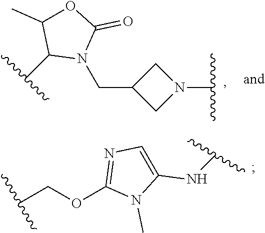

and
W is a cross-linking group selected from the following:

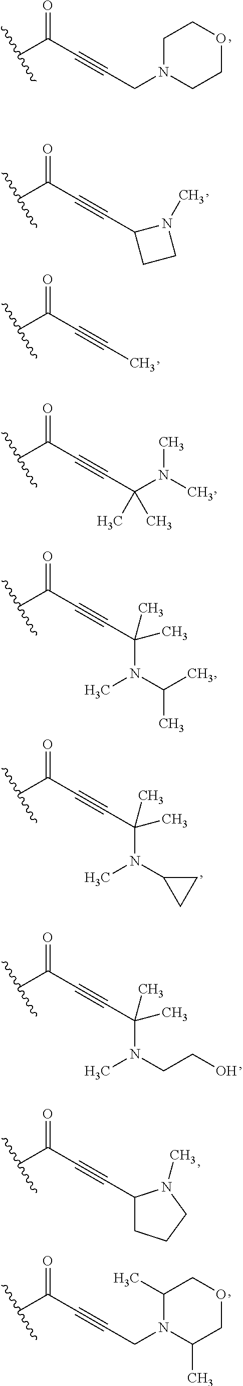

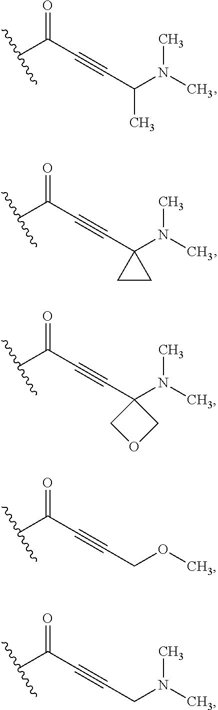

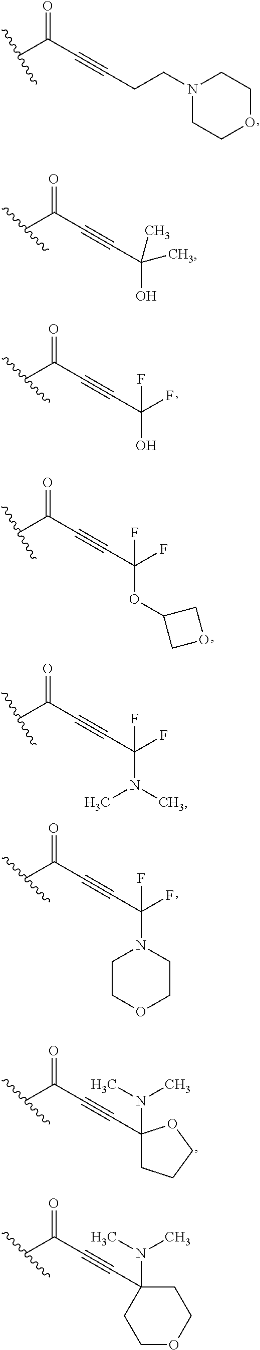

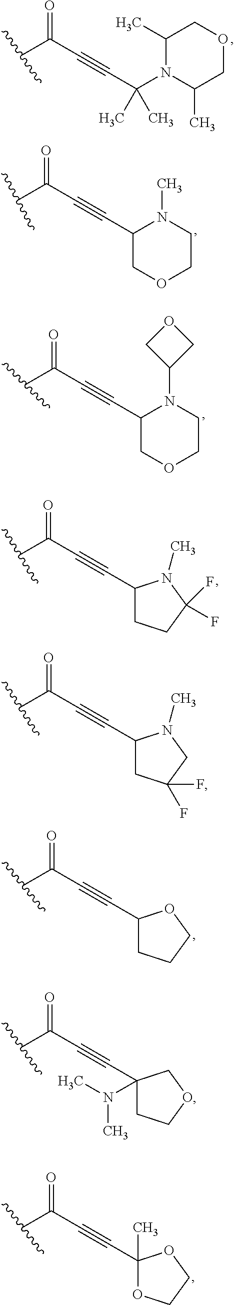

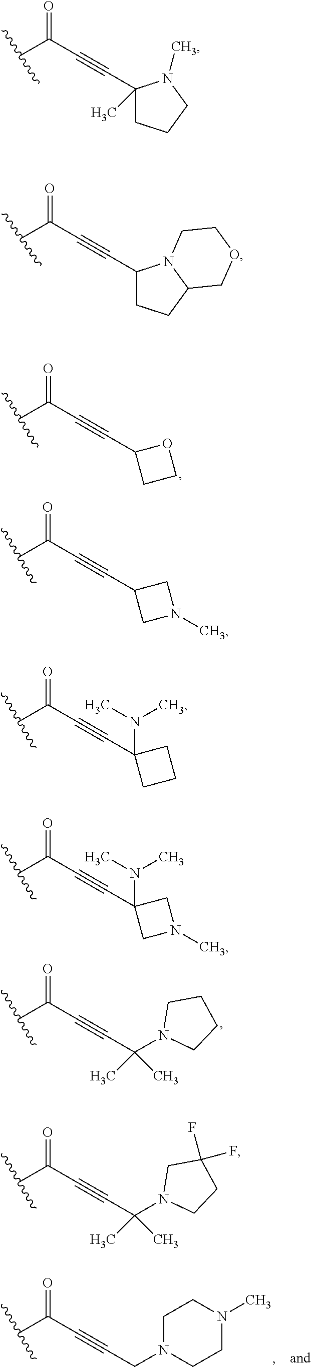

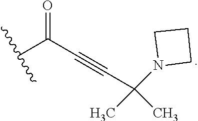 [SMILES: *C(=O)C#CC(C)(C)N1CCC1]

Embodiment IV-3. A method of treating a subject having a RAS^{G12C} mutated NSCLC or colorectal cancer, comprising administering to the subject an effective amount of RMC-5552, or a stereoisomer or tautomer thereof, in combination with a compound selected from compound A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof.
Embodiment IV-4. A method of treating a subject having a RAS^{G12C} mutated NSCLC or colorectal cancer, comprising administering to the subject an effective amount of RMC-5552, or a stereoisomer or tautomer thereof, in combination with Compound A, or a pharmaceutically acceptable salt thereof.
Embodiment IV-5. A method of treating a subject having a RAS^{G12C} mutated NSCLC or colorectal cancer, comprising administering to the subject an effective amount of RMC-5552, or a stereoisomer or tautomer thereof, in combination with Compound B, or a pharmaceutically acceptable salt thereof.
Embodiment V-1. A method of inducing apoptosis of a RAS^{G12C} mutated NSCLC or colorectal tumor cell, comprising contacting the tumor cell with an effective amount of RMC-5552, or a stereoisomer or tautomer thereof, in combination with AMG 510 or MRTX849, or a pharmaceutically acceptable salt thereof, wherein the effective amount is an amount effective to induce apoptosis of the tumor cell.
Embodiment V-2. A method of inducing apoptosis of a RAS^{G12C} mutated NSCLC or colorectal tumor cell, comprising contacting the tumor cell with an effective amount of RMC-5552, or a stereoisomer or tautomer thereof, in combination with a compound of Formula IVb of Appendix B-1, or a pharmaceutically acceptable salt thereof:

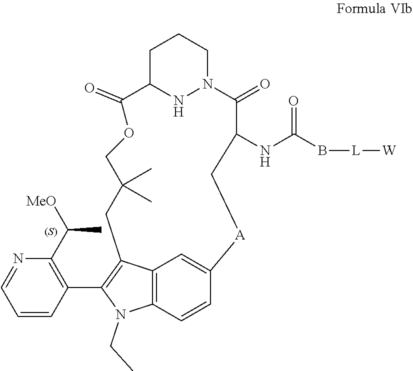

wherein A is a 3 to 6-membered heterocycloalkylene, a phenylene, or a hydroxy-substituted phenylene; B is —CH(C₁-C₆ alkyl)-; L is a linker selected from the following:

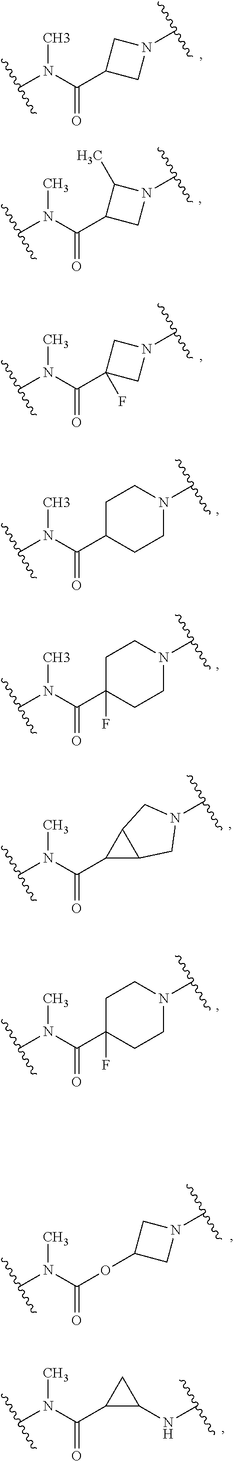

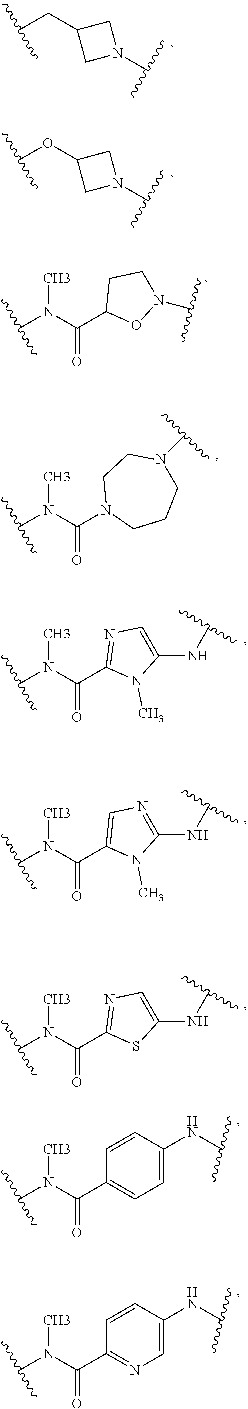

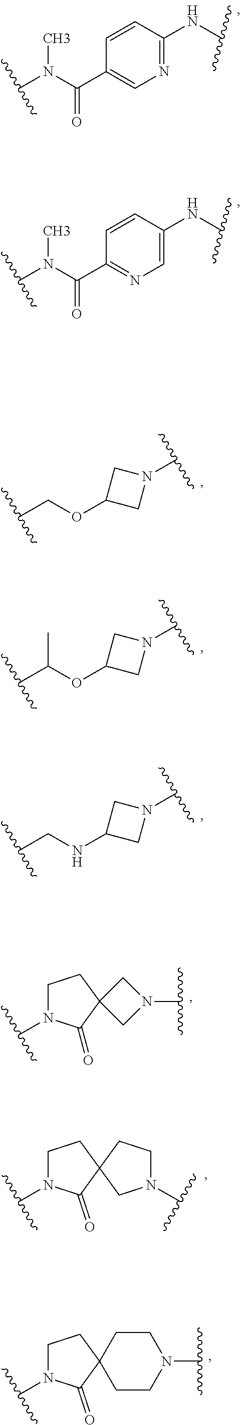

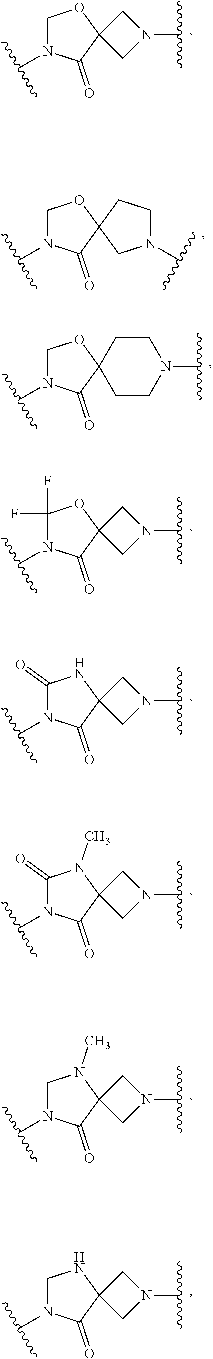

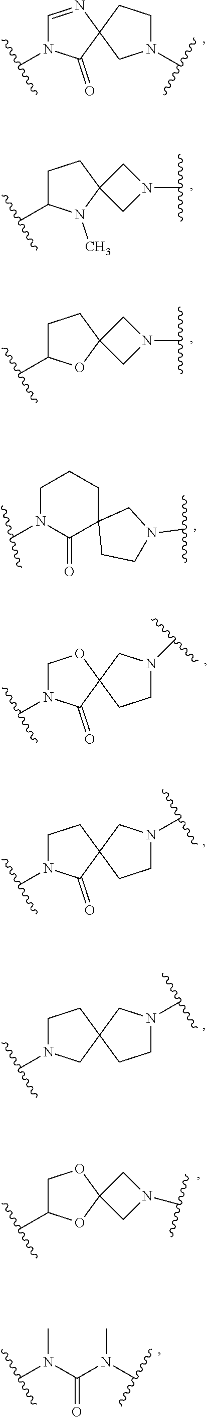

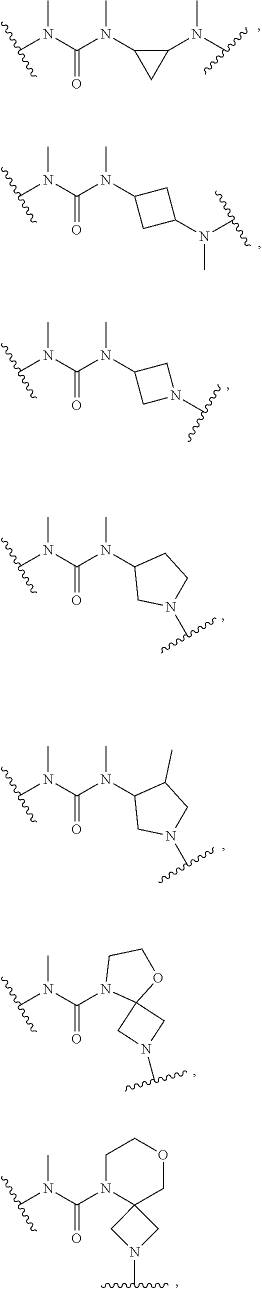

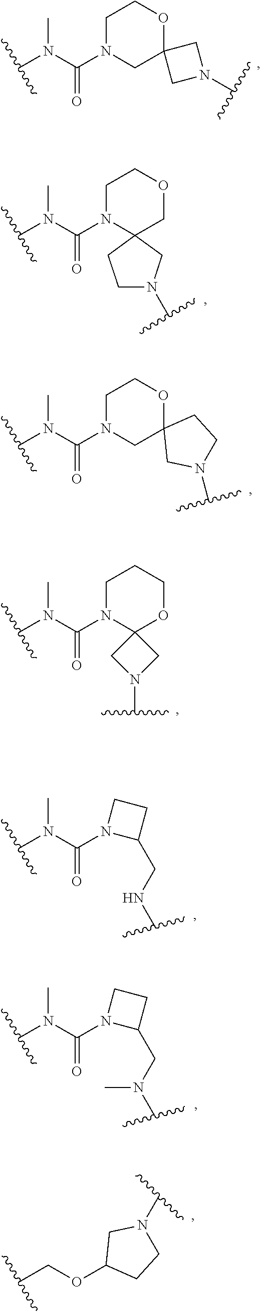

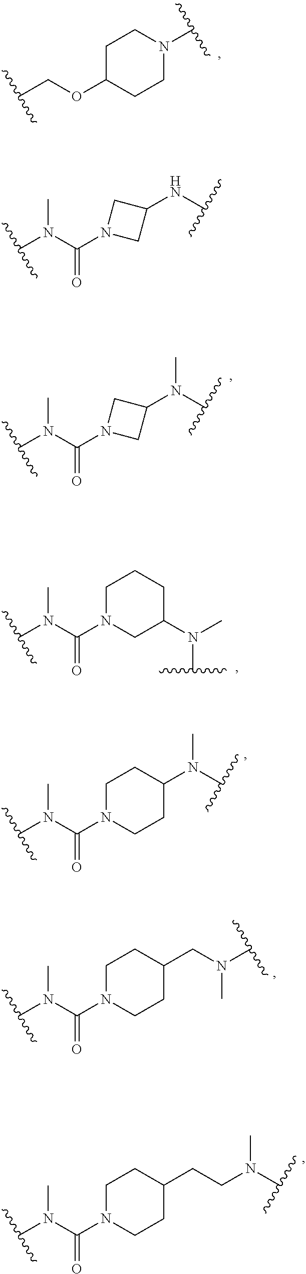

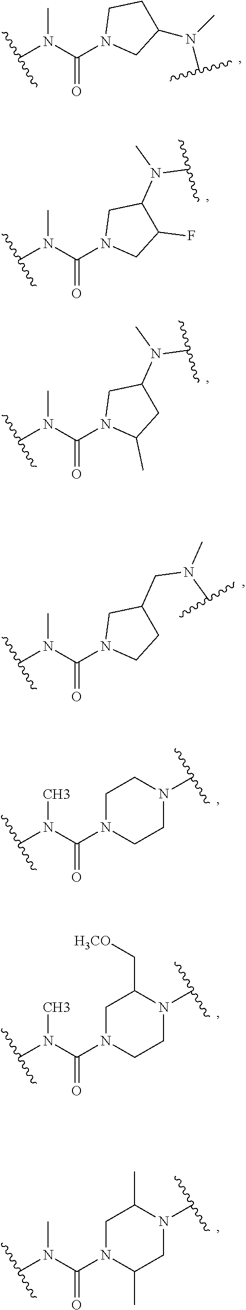

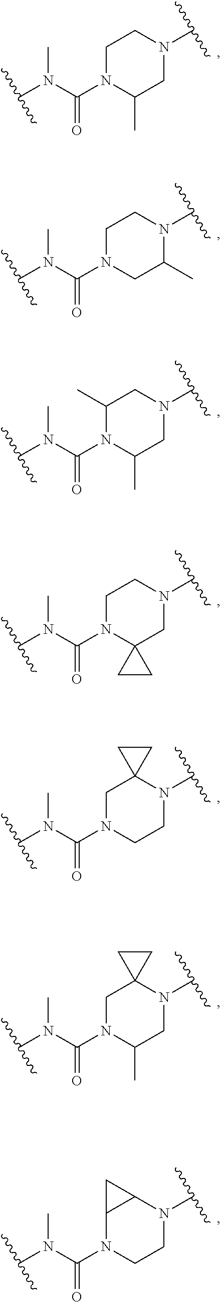

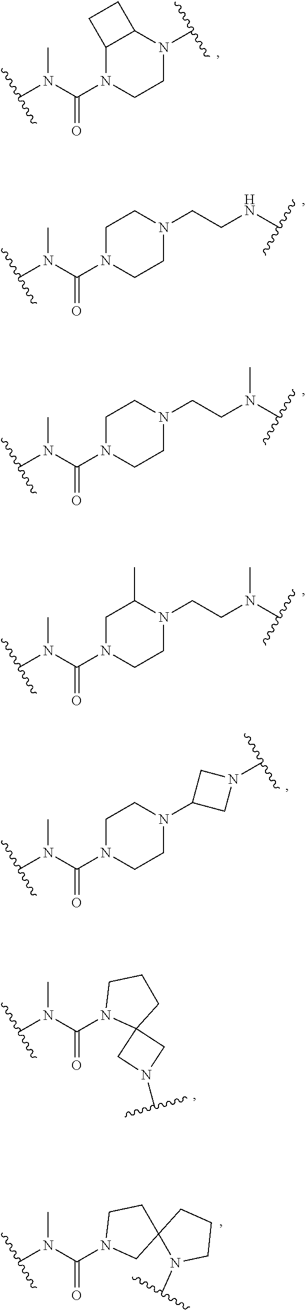

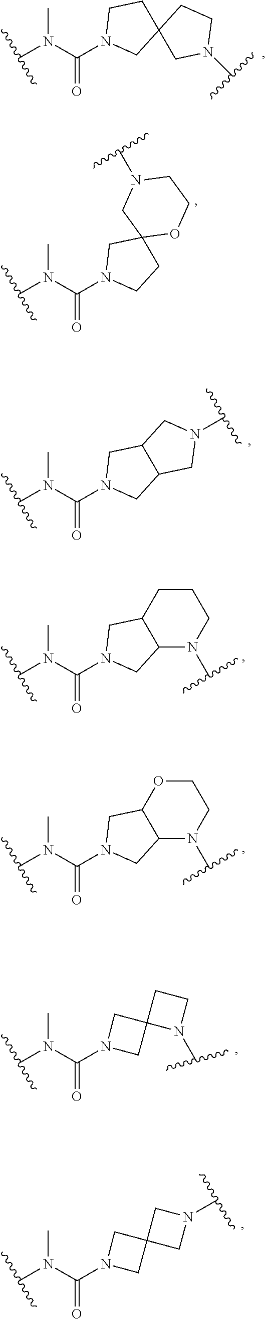

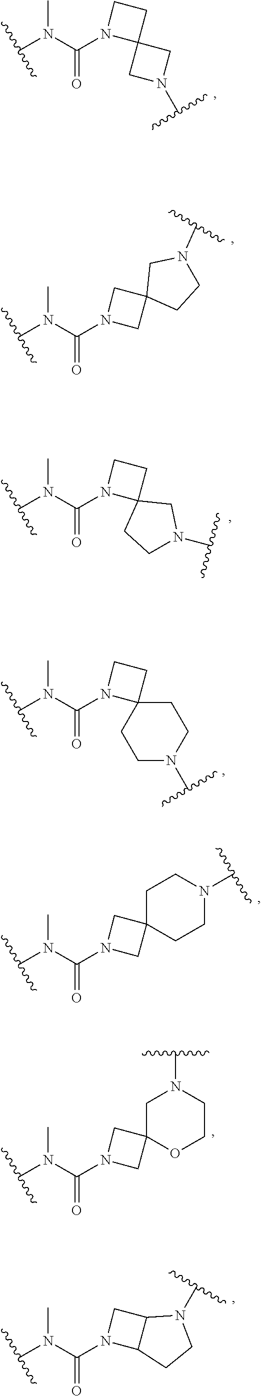

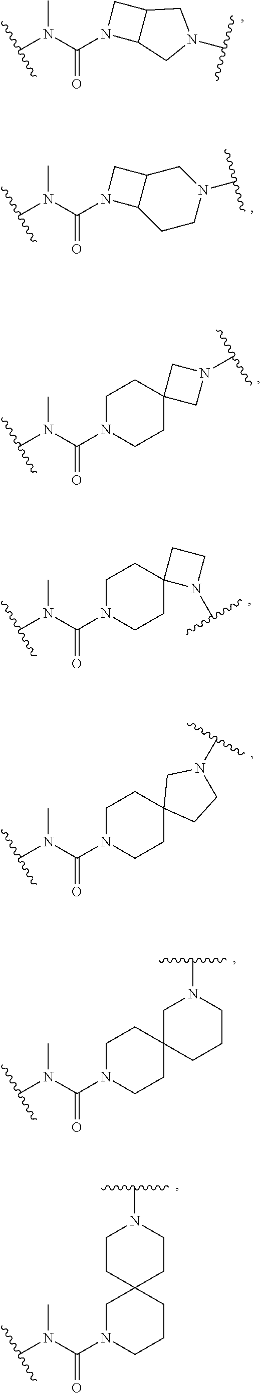

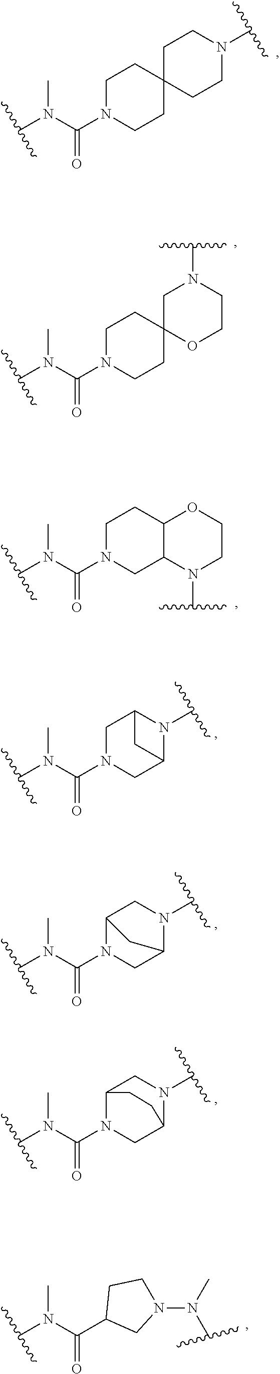

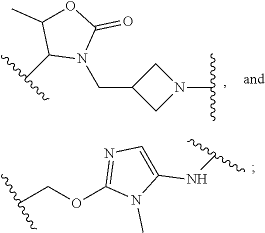

and
W is a cross-linking group selected from the following:

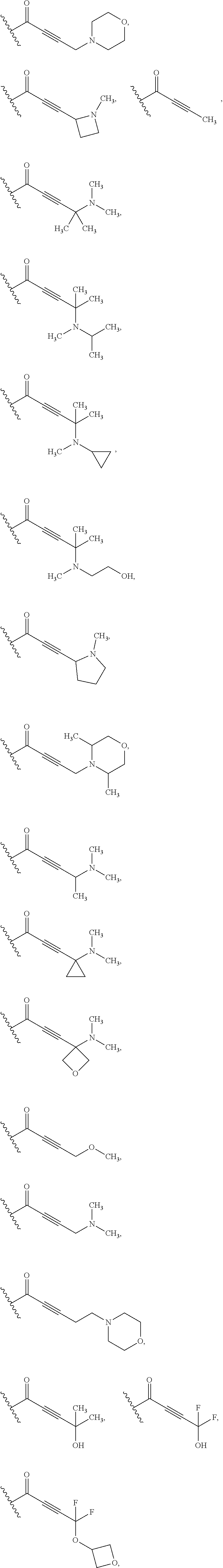

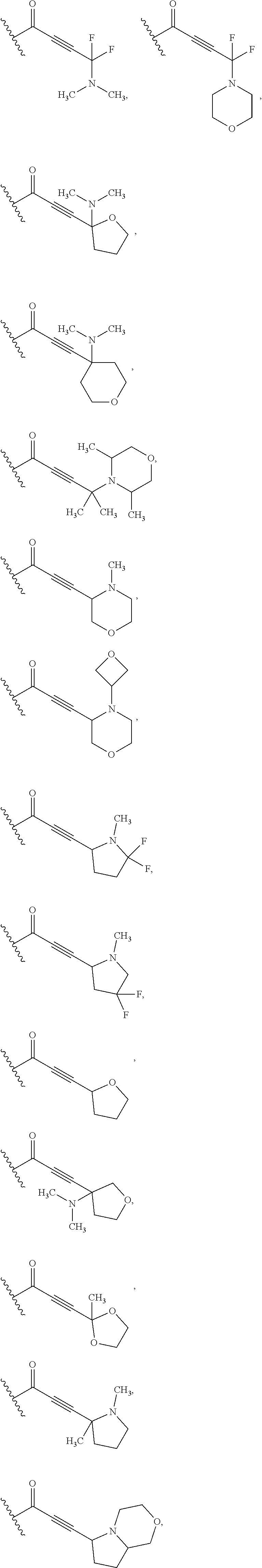

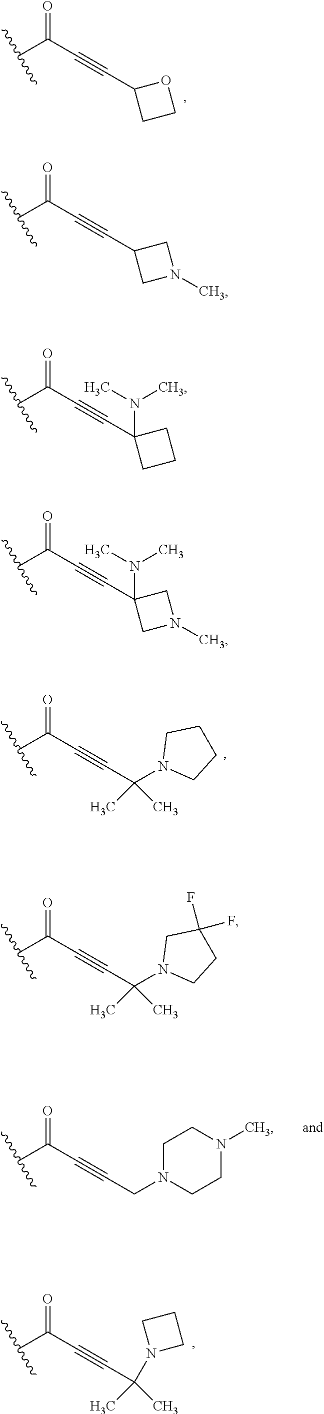

wherein the effective amount is an amount effective to induce apoptosis of the tumor cell.
Embodiment V-3. A method of inducing apoptosis of a RAS^{G12C} mutated NSCLC or colorectal tumor cell, comprising contacting the tumor cell with an effective amount of RMC-5552, or a stereoisomer or tautomer thereof, in combination with a compound selected from compound A121, A131, A133, A145, A150, A173, A182, A191, A198, A199, A201, A244, A245, A246, A247, A248, A266, A290, A292, A310, A316, A317, A324, A325, A326, A337, A339, A351, A365, A377, A391, A402, A412, A413, A414, A426, A476, A487, A499, A508, A509, A526, A528, A532, A533, A534, A551, A559, A560, A565, A566, A567, A568, A569, A584, A585, A591, A592, A599, A601, A613, A614, A615, A616, A617, A643, A644, A646, A647, A648, A657, A663, A672, A699, A708, A715, A717 and A733 of Appendix B-1, or a pharmaceutically acceptable salt thereof, wherein the effective amount is an amount effective to induce apoptosis of the tumor cell.
Embodiment V-4. A method of inducing apoptosis of a RAS^{G12C} mutated NSCLC or colorectal tumor cell, comprising contacting the tumor cell with an effective amount of RMC-5552, or a stereoisomer or tautomer thereof, in combination with Compound A, or a pharmaceutically acceptable salt thereof, wherein the effective amount is an amount effective to induce apoptosis of the tumor cell.
Embodiment V-5. A method of inducing apoptosis of a RAS^{G12C} mutated NSCLC or colorectal tumor cell, comprising contacting the tumor cell with an effective amount of RMC-5552, or a stereoisomer or tautomer thereof, in combination with Compound B, or a pharmaceutically acceptable salt thereof, wherein the effective amount is an amount effective to induce apoptosis of the tumor cell.

All of the U.S. patents, U.S. patent application publications, U.S. patent applications, PCT patent application, PCT patent application publications, foreign patents, foreign patent applications and non-patent publications referred to in this specification or listed in any Application Data Sheet are incorporated herein by reference in their entirety. From the foregoing it will be appreciated that, although specific embodiments of the invention have been described herein for purposes of illustration, various modifications may be made without deviating from the spirit and scope of the invention.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

In Vitro Combinatorial Activity of RM-006 (also Known as RMC-6272) and KRAS^{G12C}(OFF) Inhibitor in NSCLC Cells with RAS & mTOR Signaling Co-Activation

### Objective:

The RAS and PI3K/mTOR signaling pathways are hyperactivated in many human cancers. In the PI3K/mTOR pathway, mTORC1 phosphorylates and inactivates the tumor suppressor 4EBP1, enabling cap-dependent translation, including translation of key oncogenes. We have developed a series of bi-steric mTORC1-selective inhibitors that activate 4EBP1. As shown in Table 1, RM-006 (also known as RMC-6272), one representative example of these new bi-steric inhibitors, has potent and selective (>10 fold) inhibition of mTORC1 over mTORC2, and durably suppress phosphorylation of S6K and 4EBP1 in vitro and in vivo.

TABLE 1

| | Inhibition of Substrate Phosphorylation | | | |
|---|---|---|---|---|
| | (IC₅₀, nM) in MDA-MB-468 (PTEN^{null}, | | | |
| | EGFR^{amp}) Breast Cancer Cells | | | |
| | pS6K | p4EBP1 | pAKT | mTORC1/2 |
| Compound | mTORC1 | mTORC1 | mTORC2 | selectivity ratio* |
| Rapamycin | 0.063 | n/a | n/a | n/a |
| MLN0128 | 0.683 | 18.90 | 1.77 | 0.09 |
| RM-006 | 0.15 | 0.44 | 11.8 | 26.7 |

*IC₅₀ pAKT/IC₅₀ p4EBP1

In this Example, we tested the in vitro combinatorial effect of the bi-steric mTOR inhibitor RM-006 (also known as RMC-6272) and the KRAS^{G12C}(OFF) inhibitor AMG 510 on non-small cell lung cancer cell lines NCI-H2122 and NCI-H2030, which each have a KRAS^{G12C} mutation and mTOR signaling co-activation.

### Methods:

Cells were grown in culture as 3D spheroids. Briefly, 1000 cells/well (for NCI-H2122) and 1500 cells/well (for NCI-H2030) were seeded in round bottom ultra-low attachment 384-well plates in growth media supplemented with 10% fetal bovine serum and 1% penicillin/streptomycin, and allowed to form spheroids for 24 hours at 37° C. in 5% CO2. Spheroid formation was confirmed visually, and spheroids were treated in duplicate with serial 3.16-fold dilutions of single-agent inhibitor or in combination (final DMSO concentration=0.2%). Following drug exposure for five days, cell viability in spheroids was determined using the 3D-CellTiter-Glo® assay kit (Promega).

### Results:

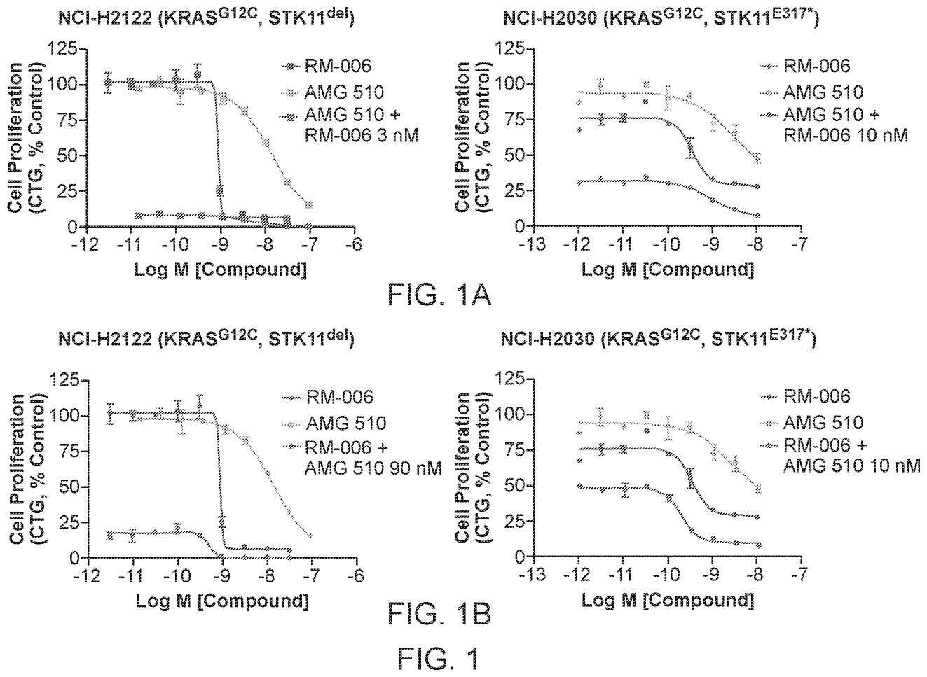

RM-006 (also known as RMC-6272) shows in vitro combinatorial anti-proliferative activity with AMG 510 in two NSCLC cell lines with co-occurring KRAS^{G12C} and STK11 loss of function mutations. STK11 is a negative regulator of mTOR signaling. In FIG. 1A, we evaluated varying concentrations of AMG 510 in presence of constant RM-006 (also known as RMC-6272) (3 nM in H2122, left panel, and 10 nM in H2030, right panel), and showed combinatorial anti-proliferative activity at select concentrations of AMG 510. In FIG. 1B, we evaluated varying concentrations of RM-006 (also known as RMC-6272) in presence of constant AMG 510 (90 nM in H2122, left panel, or 10 nM in H2030, right panel), and showed combinatorial anti-proliferative activity at select concentrations of RM-006 (also known as RMC-6272). Thus, the combination of a bi-steric mTORC1-selective inhibitor with a KRAS inhibitor drives tumor regression in a NSCLC model with co-activation of RAS & mTOR signaling.

### Example 2

In Vivo Combinatorial Activity of RM-006 (Also Known as RMC-6272) and KRAS^{G12C}(OFF) Inhibitor in Non-Small Cell Lung Cancer NCI-11358 KRAS^{G12C} Xenograft Model

### Objective:

Having demonstrated in Example 1 that the combined inhibition of the RAS and PI3K/mTOR signaling pathways provided for significant in vitro anti-tumor activity, we sought to extend our results to an in vivo tumor model. To that end, the combinatorial effects of RM-006 (also known as RMC-6272) with AMG 510 on tumor cell growth in vivo were evaluated in the human non-small cell lung cancer NCI-H358 KRAS^{G12C} xenograft model using female BALB/c nude mice (6-8 weeks old).

### Methods:

Mice were implanted with NCI-H358 tumor cells in 50% Matrigel (5×106 cells/mouse) subcutaneously in the flank. Once tumors reached an average size of ˜200 mm3, mice were randomized to treatment groups to start the administration of test articles or vehicle. RM-006 (also known as RMC-6272) was administered by intraperitoneal injection once weekly, and AMG 510 was administered by oral gavage daily. Body weight and tumor volume (using calipers) was measured twice weekly until study endpoints.

### Results:

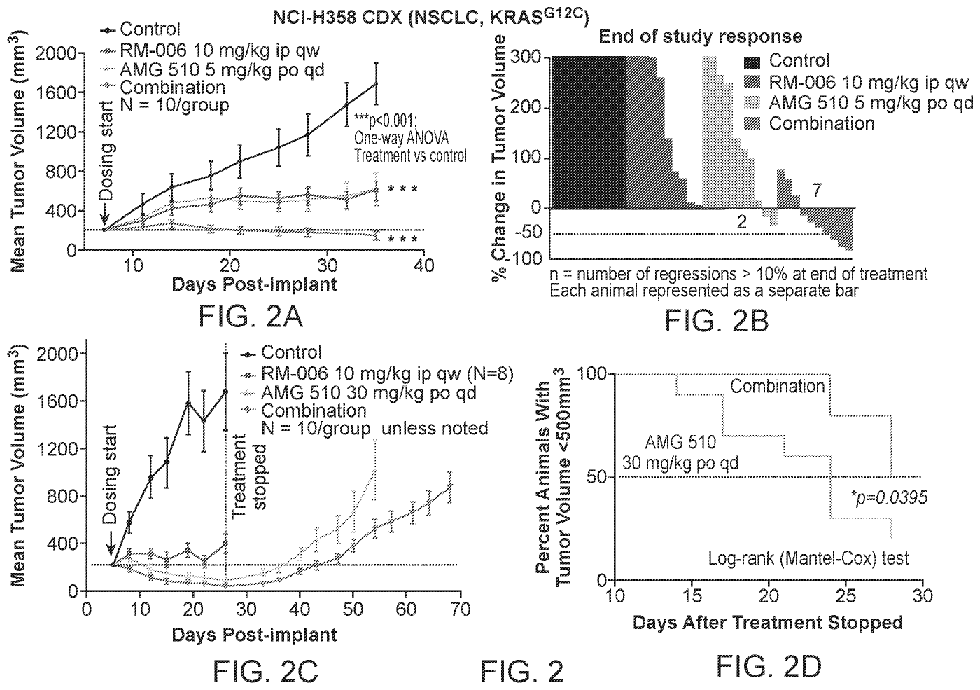

FIG. 2A shows a mean tumor volume plot and demonstrates that the combination of RM-006 (also known as RMC-6272) administered at 10 mg/kg IP weekly plus AMG 510 given at a submaximal dose of 5 mg/kg via PO daily led to tumor regression in NCI-H358 KRAS^{G12C} xenograft model, which is a sensitive model to KRAS^{G12C} inhibition alone. The end of study responses of each mouse represented as a waterfall plot is shown in FIG. 2B. The number of tumors with reduction in tumor volume greater than 10% of the baseline is indicated on the waterfall plot (FIG. 2B).

In FIG. 2C, the combination of RM-006 (also known as RMC-6272) at 10 mg/kg IP weekly plus AMG 510 inhibitor at 30 mg/kg PO daily (a regression-driving dose but submaximal) resulted in a more durable response that delayed tumor regrowth after treatment cessation, relative to AMG 510 alone. Kaplan-Meier analysis shown in FIG. 2D demonstrates that when comparing to the single-agent AMG 510, combination with RM-006 (also known as RMC-6272) significantly delayed the regrowth of tumors back to 500 mm3 after treatment cessation, as assessed by Log-rank (Mantel-Cox) test with p=0.0395. The combination treatment was well tolerated. These findings indicate that even in mutant KRAS tumor cells without known genomic aberrations conferring mTOR pathway activation, the concomitant targeting of both the RAS and mTOR signaling by an inhibitor of mutant KRAS and a bi-steric mTORC1-selective inhibitor may demonstrate benefit over mutant KRAS inhibitor alone.

### Example 3

In Vivo Combinatorial Activity of RM-006 (Also Known as RMC-6272) and KRAS^{G12C}(OFF) Inhibitor in Non-Small Cell Lung Cancer NCI-112122 KRAS^{G12C}; STK11del Xenograft Model

### Objective:

To further explore the in vivo utility that the combined inhibition of the RAS and PI3K/mTOR signaling pathways provides, we investigated the combinatorial effects of RM-006 (also known as RMC-6272) with AMG 510 on tumor cell growth in vivo in the human non-small cell lung cancer NCI-H2122 KRAS^{G12C}; STK11del xenograft model using female BALB/c nude mice (6-8 weeks old).

### Methods:

Mice were implanted with NCI-H2122 tumor cells in 50% Matrigel (5×106 cells/mouse) subcutaneously in the flank. Once tumors reached an average size of ˜166 mm3, mice were randomized to treatment groups to start the administration of test articles or vehicle. RM-006 (also known as RMC-6272) was administered by intraperitoneal injection once weekly, and AMG 510 was administered by oral gavage daily. Body weight and tumor volume (using calipers) was measured twice weekly until study endpoints.

### Results:

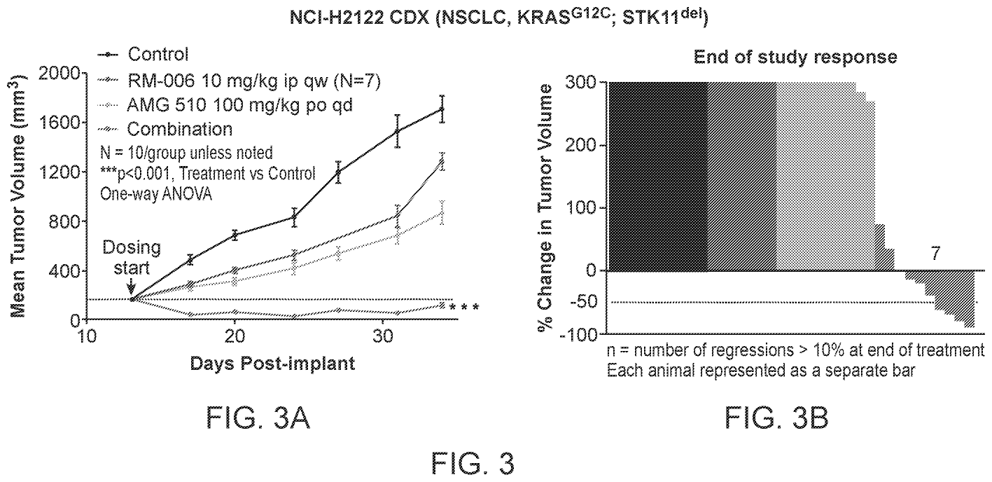

As shown in the tumor volume plot in FIG. 3A, single-agent RM-006 (also known as RMC-6272) administered at 10 mg/kg IP weekly led to a tumor growth inhibition (“TGI”) of 27.4%, and single-agent AMG 510 administered at 100 mg/kg PO daily led to a TGI of 54.6% in the NCI-H2122 NSCLC CDX model with co-occurring KRAS^{G12C} and STK11de. However, the combination drove tumor regressions in the NCI-H2122 model. The anti-tumor activity by the combination treatment was statistically significant from the vehicle control group, with ***p<0.001, assessed by an ordinary One-way ANOVA of tumor volumes along with multiple comparisons via a post-hoc Tukey's test in GraphPad Prism software. In FIG. 3B, waterfall plot shows individual tumor responses at the end of study, and 7/10 tumors from the combination group showed reductions in tumor volume greater than 10% of the baseline. The combination treatment was well tolerated. These data demonstrate that the Combination of RM-006 (also known as RMC-6272) and KRAS^{G12C}(OFF) inhibition drives tumor regression in a NSCLC model with co-activation of RAS & mTOR signaling.

The NCI-H2122 model is an example of a NSCLC model that exhibited relatively lower anti-tumor response to either KRAS^{G12C}(OFF) inhibitor or mTORC1 inhibitor monotherapy, as evidenced by some tumor growth inhibition but no reductions in tumor volume in preclinical studies. In contrast, the combination of both inhibitors resulted in tumor regressions and exemplifies the use of this therapeutic regimen to overcome up-front or intrinsic resistance. NCI-H2122 tumor cells harbor activating mutations that drive oncogenic signaling via both the RAS and the mTOR signaling pathway. Thus, we hypothesize that neither single agent is able to sufficiently overcome the oncogenic flux driven by the co-activation of both pathways and combination therapy is required to induce apoptosis and tumor regressions.

### Example 4

In Vivo Single-Dose PKPD Study of the Combinatorial Activity of RM-006 (Also Known as RMC-6272) and KRAS^{G12C}(OFF) Inhibitor in Human Non-Small Cell Lung Cancer NCI-H2122 KRAS^{G12C}; STK11^{del} Xenograft Model

### Objective:

We investigated the pharmacokinetic and pharmacodynamic (PKPD) effects RM-006 (also known as RMC-6272), AMG-510, and the combination of the two inhibitors had in human non-small cell lung cancer NCI-H2122 KRAS^{G12C}; STK11del xenograft model.

### Methods:

RM-006 (also known as RMC-6272) was administered at 10 mg/kg IP, whereas AMG 510 was administered at 100 mg/kg by oral gavage. The treatment groups with sample collections at various time points were summarized in Table 1 below. Plasma samples were collected for bioanalysis of the compounds, and tumor samples were collected to assess pathway modulation by quantitative image analyses of immunohistochemical (IHC) staining for phosphorylated proteins that are known biomarkers of mTOR and RAS pathway activity. Tumor sections were stained with monoclonal antibodies against pS6RP(Ser235/236), p4E-BP1(Thr37/46), and pERK (Thr202/Tyr204), and visualized with DAB chromogen and, counterstained with hematoxylin, and scanned to generate a digital image. Digital images were analyzed with Indica Lab's HALO software using the area quantification module where colors and intensity were measured on a pixel by pixel basis. Whole tumor sections, excluding necrotic regions and murine tissue, were measured for intensity above background and the percent positivity calculated for the given area measured. Additionally, qPCR assay was used to measure the mRNA level of human DUSP6 as another marker for RAS/ERK signaling.

The treatment groups, doses, and time points for the single-dose PKPD study using NCI-H2122 tumors are shown in Table 2.

TABLE 2
Summary of treatment groups, doses, and time points
for single-dose PKPD study using NCI-H2122 tumors.

| | | | PK, n = 3/ | PD, n = 3/ |
|---|---|---|---|---|
| Compound/group | Dose | | time point | time point |
| Vehicle control | 10 | ml/kg | 1 h, 24 h | 1 h, 24 h |
| RM-006 (also | 10 | mg/kg | 0.5 h, 1 h, 4 h, | 1 h, 4 h, 6 h, |
| known as RMC- | | | 6 h, 24 h, 48 h | 24 h, 48 h |
| 6272) | | | | |
| AMG 510 | 100 | mg/kg | 0.5 h, 1 h, 4 h, | 1 h, 4 h, 6 h, |
| | | | 6 h, 24 h, 48 h | 24 h, 48 h |
| Combination | 10 + 100 | mg/kg | 0.5 h, 1 h, 4 h, | 1 h, 4 h, 6 h, |
| | | | 6 h, 24 h, 48 h | 24 h, 48 h |

### Results:

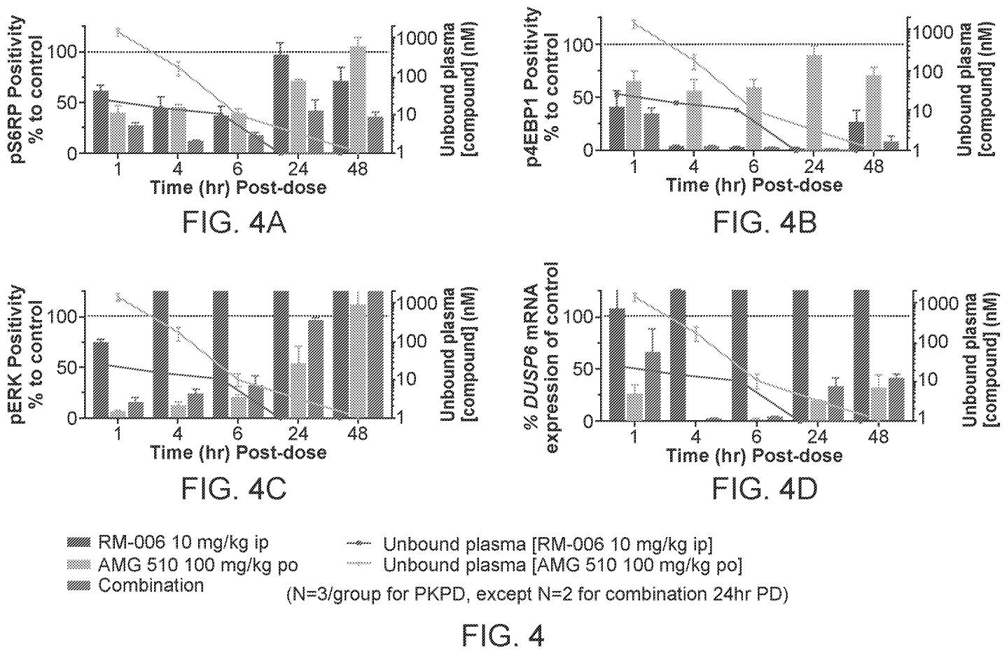

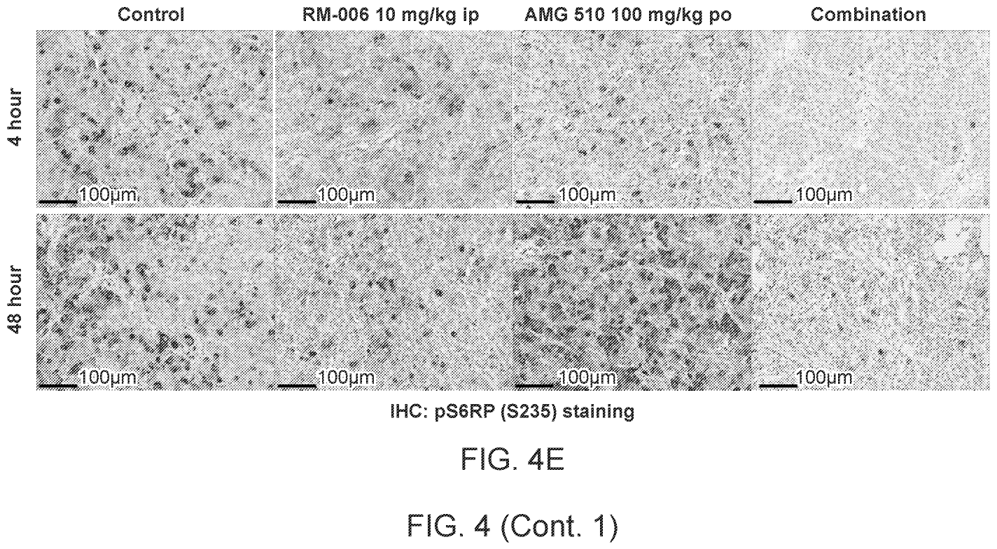

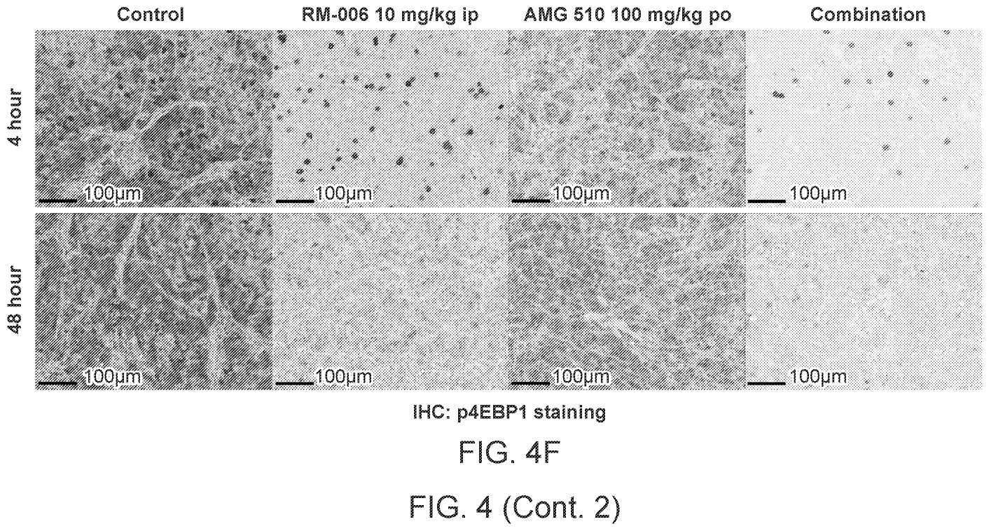

As shown in FIG. 4A, the combination of RM-006 (also known as RMC-6272) at 10 mg/kg IP and AMG 510 100 mg/kg PO led to stronger inhibition of pS6RP (Ser235/236) across all time points, relative to each single agent. pS6RP (Ser235/236) is a key converging node that can be modulated by both the mTOR and RAS pathways. As shown in FIGS. 4B-4D, the effects on p4EBP1, pERK, and DUSP6 are consistent with the expected pathway modulation by RM-006 (also known as RMC-6272) and AMG 510 on mTOR and RAS signaling, respectively. Unbound plasma concentration of each single agent is shown as lines on the bar graphs of FIGS. 4A-4D. In combination, the PK profile of each agent is consistent with that of the single agent, indicative of no DDI, thus only single agent PK is shown. Representative IHC staining images for pS6RP (Ser235/236) and p4EBP1 (Thr37/46) at 4 and 48 hours after dosing are shown in FIGS. 4E and 4F, respectively.

Tumors from the single-dose study described in FIG. 4 were stained for cleaved caspase3 (CC3) using a polyclonal antibody by IHC. HALO quantitative image analysis as described above was applied to assess the overall CC3 induction.

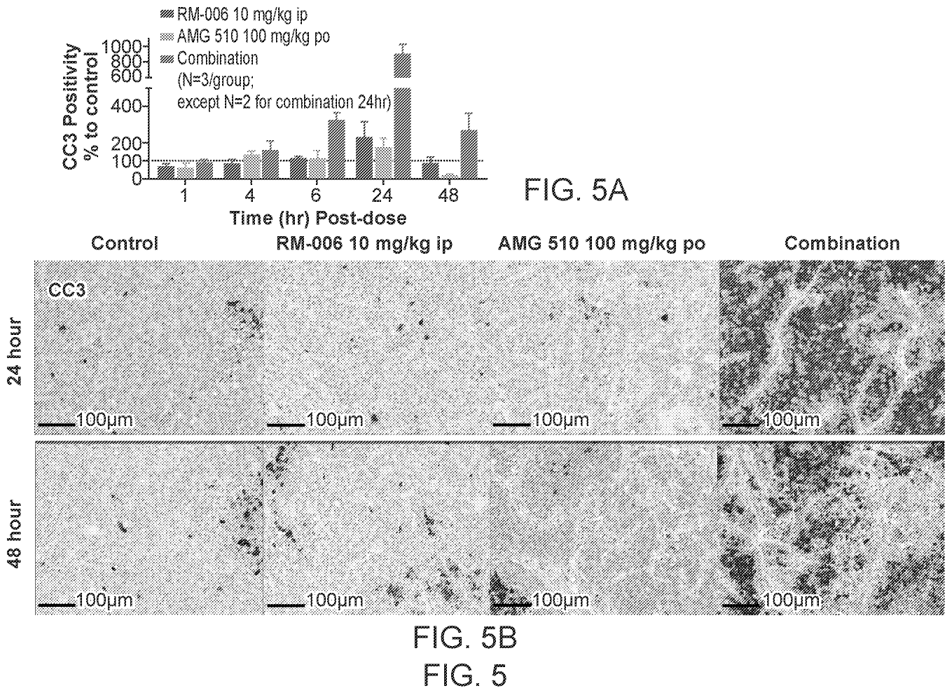

As shown in FIGS. 5A and 5B, in the human non-small cell lung cancer NCI-H2122 KRAS^{G12C}; STK11^{del} tumors, combination of RM-006 (also known as RMC-6272) and AMG 510 led to a significant induction of apoptosis as measured by cleaved caspase 3 IHC staining, relative to each single agent alone. Based on the time points we assessed, maximal induction of apoptosis occurred at 24 hours after a single dose, resulting in an induction of CC3 positivity ˜900% more than the control group. The % CC3 positivity of tumors from each treatment group at various time points is summarized as mean with SEM in Table 3 below. These values were used to generate the bar graph shown in FIG. 5A.

TABLE 3
Percent CC3 positivity of tumors from each treatment groups
at indicated time points is summarized as mean and SEM
with N indicating the number of mice. The values from this
table were used to generate the bar graph in FIG. 5A.

| | % CC3 positivity normalized to control group | | | | | | | | |
|---|---|---|---|---|---|---|---|---|---|
| | RMC-6272* | | | AMG 510 | | | | | |
| Time (hr.) | 10 mg/kg ip | | | 100 mg/kg po | | | Combination | | |
| after dose | Mean | SEM | N | Mean | SEM | N | Mean | SEM | N |
| 1 | 71.3 | 10.7 | 3 | 60.7 | 26.0 | 3 | 93.0 | 12.9 | 3 |
| 4 | 86.3 | 20.3 | 3 | 134.0 | 18.3 | 3 | 160.3 | 46.0 | 3 |
| 6 | 115.7 | 6.1 | 3 | 111.7 | 44.5 | 3 | 326.0 | 38.5 | 3 |
| 24 | 232.3 | 81.2 | 3 | 172.0 | 53.4 | 3 | 915.0 | 115.0 | 2 |
| 48 | 87.0 | 32.1 | 3 | 20.0 | 3.0 | 3 | 268.7 | 92.3 | 3 |

*also known as RM-006

Adult somatic cells almost all will die by apoptosis, a form of programmed cell death. Cancer cells, harboring alterations that result in impaired apoptotic signaling, often acquire the ability to evade death by inactivating cell death pathways (Long 2012). Hence, reduced apoptosis or its resistance plays a vital role in carcinogenesis (Hanahan 2000).

A successfully cancer therapy can promote cancer cell death while minimizing comparable damage to normal cells. Numerous in vitro and in vivo studies have indicated that tumor cell apoptosis induction is part of the mechanism of action of many approved drugs in cancer treatment in both preclinical and clinical settings (Gert 2005).

In this study, our results demonstrate that combination treatment of RM-006 (also known as RMC-6272) and KRAS^{G12C} inhibitor can induce significant apoptosis in NCI-H2122 xenograft tumors in vivo. This is the first time to our knowledge that combination treatment with an mTOR inhibitor and KRAS^{G12C} mutant selective inhibitor has shown to promote tumor apoptosis in vivo.

### REFERENCES

Hanahan D, Weinberg R A. The hallmarks of cancer. Cell. 2000; 100:57-70.
Gerl R, Vaux D L. Apoptosis in the development and treatment of cancer, Carcinogenesis. 2005; 26(2):263-270
Long J, Ryan, K. New frontiers in promoting tumor cell death: targeting apoptosis, necroptosis and autophagy. Oncogene 2012; 31:5045-5060.

### Example 5

Combination of RM-006 (Also Known as RMC-6272) and a KRAS^{G12C}(OFF) Inhibitor Significantly Delays On-Treatment Resistance in a NSCLC Model with RAS and mTOR Signaling Co-Activation

### Objective:

We investigated the in vivo combinatorial effects of RM-006 (also known as RMC-6272) with AMG 510 on tumor cell growth in the human non-small cell lung cancer NCI-H2030 KRAS^{G12C}; STK11E317* xenograft model using female NOD SCID mice (4-5 weeks old).

### Methods:

Mice were implanted with NCI-H2030 tumor cells in 50% Matrigel (1×107 cells/mouse) subcutaneously in the flank. Once tumors reached an average size of 150-200 mm3, mice were randomized to treatment groups to start the administration of test articles or vehicle. RM-006 (also known as RMC-6272) was administered by intraperitoneal injection once weekly, and AMG 510 was administered by oral gavage daily. Body weight and tumor volume (using calipers) was measured twice weekly until study endpoints.

### Results:

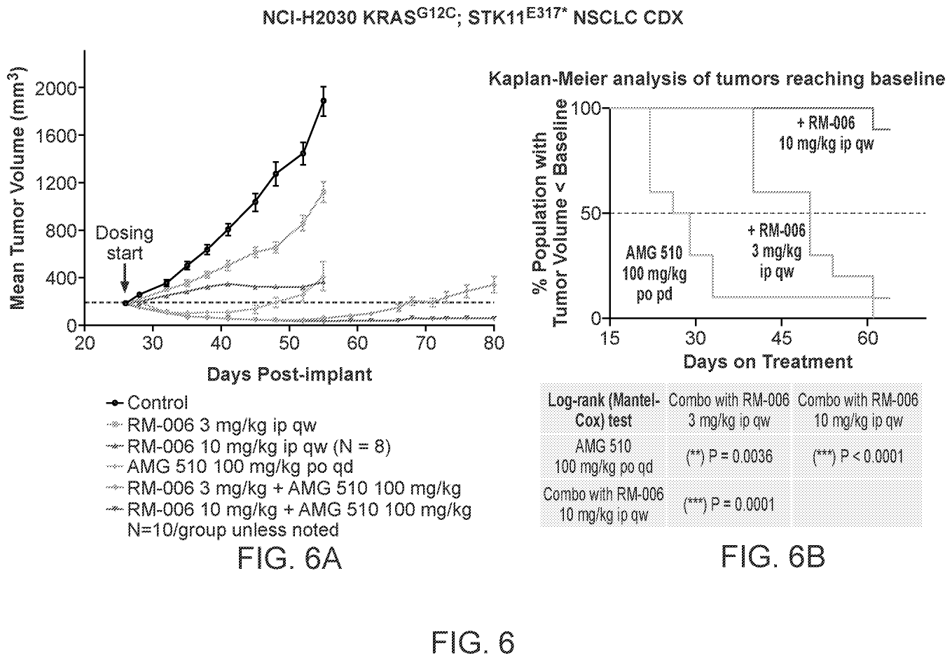

In the human non-small cell lung cancer NCI-H2030 KRAS^{G12C}; STK11^{E317}* tumors, the combination of RM-006 (also known as RMC-6272) dosed at 3 mg/kg or 10 mg/kg IP weekly plus AMG 510 100 mg/kg PO daily resulted in durable tumor regression and delayed on-treatment resistance, relative to single agent AMG 510, as shown by the mean tumor volume plot presented in FIG. 6A. Kaplan-Meier analysis of tumors reaching baseline volume while on treatment showed the combination significantly prolonged the time for tumors to develop resistance, as assessed by Log-rank (Mantel-Cox) test (FIG. 6B). Table 4 below summarizes the comparisons and P values.

TABLE 4
Comparisons of treatment groups and summary of P values by Log-
rank (Mantel-Cox) test, where RM-006 is also known as RMC-6272.

| | Combo | Combo |
|---|---|---|
| | with RM-006 | with RM-006 |
| Log-rank (Mantel-Cox) test | 3 mg/kg ip qw | 10 mg/kg ip qw |
| AMG 510 100 mg/kg po qd | (**) P = 0.0036 | (***) P < 0.0001 |
| Combo with RM-006 | (***) P = 0.0001 | |
| 10 mg/kg ip qw | | |

### Example 6

Combination of RM-006 (Also Known as RMC-6272) and a KRAS^{G12C}(OFF) Inhibitor Attenuates AMG 510 On-Treatment Resistant Tumor Growth in the Human Non-Small Cell Lung Cancer NCI-112030 KRAS^{G12C}; STK11E³¹⁷* Xenograft Model

### Objective:

We evaluated whether combination treatment of RM-006 (also known as RMC-6272) and AMG 510 could attenuate AMG 510 on-treatment resistant tumor growth in the human non-small cell lung cancer NCI-H2030 KRAS^{G12C}; STK11^{E317*} xenograft model after the development of resistance.

### Methods:

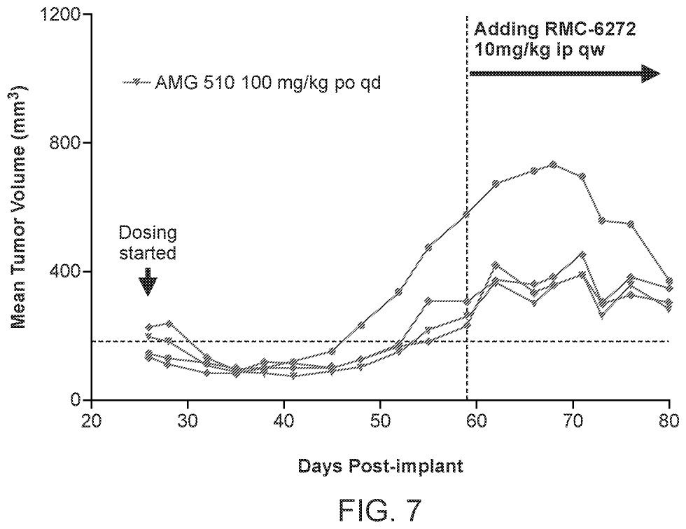

At Day 59 post-implantation in the experiment described in Example 6, above, animals treated with AMG 510 administered by oral gavage daily at 100 mg/kg exhibited on-treatment resistance (see Figure. 7). At this time, RM-006 was administered to the same group of animals by intraperitoneal injection once weekly at 10 mg/kg, while AMG 510 treatment continued. Body weight and tumor volume (using calipers) was measured twice weekly until study endpoints.

### Results:

In the human non-small cell lung cancer NCI-H2030 KRAS^{G12C}; STK11^{E317*} tumors, AMG 510 100 mg/kg PO daily treatment group developed on-treatment resistance after 2-3 weeks of treatment (FIG. 7). Adding RM-006 (also known as RMC-6272) (10 mg/kg IP weekly) in combination with AMG 510 (100 mg/kg PO daily) to the same group of animals attenuated resistant tumor growth, as shown by the individual tumor volume plot (FIG. 7).

The NCI-H2030 model exemplifies a scenario wherein a KRAS^{G12C} mutant tumor is initially sensitive to KRAS^{G12C}(OFF) inhibitor monotherapy, as demonstrated by the initial tumor regressions observed following treatment in this model. However, upon longer-term treatment, xenograft tumors were able to regrow and exhibited on-treatment resistance. The combination of KRAS^{G12C}(OFF) inhibitor and mTORC1 inhibitor significantly delayed the onset of this on-treatment resistance. Moreover, the addition of an mTORC1 inhibitor to KRAS^{G12C}(OFF) inhibitor treatment at the onset of monotherapy resistance (to the latter) resulted in attenuation of tumor growth and in some cases, apparent regression following combination therapy.

In sum, these results support that mTOR activation limits therapeutic response to mutant KRAS^{G12C} inhibition; and provide an initial demonstration that combinatorial inhibition of RAS and mTOR signaling is sufficient to forestall on-treatment resistance to KRAS^{G12C}(OFF) inhibition.

### Example 7

Combination of RM-006 (Also Known as RMC-6272) and a KRAS^{G12C}(OFF) Inhibitor Attenuates AMG 510 on Tumor Growth in the Human Colorectal Cancer (CRC) Patient Derived Xenograft (PDX) ST3235 (PIK3CA^{E545K}) Model

### Objective:

We evaluated whether combination treatment of RM-006 (also known as RMC-6272) and AMG 510 could attenuate AMG 510 on tumor growth in the human colorectal cancer (CRC) patient derived xenograft (PDX) ST3235 (PIK3CA^{E545K}) model after the development of resistance.

### Methods:

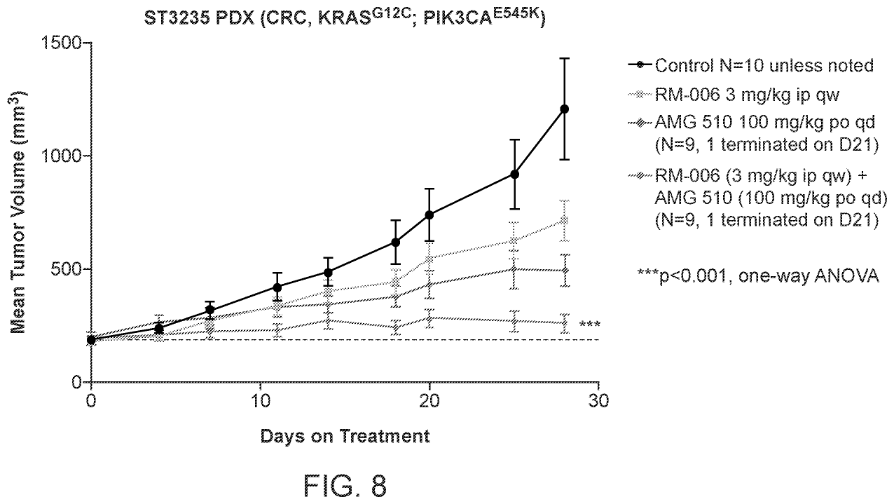

The combinatorial effects of RM-006 (also known as RMC-6272) with AMG 510 on tumor growth in vivo were evaluated in the human colorectal cancer (CRC) patient derived xenograft (PDX) model ST3235 KRAS^{G12C}; PIK3CA^{E545K} using female athymic nude mice (6-12 weeks old) (FIG. 8). Tumor fragments of about 70 mg in weight from ST3235 CRC PDX model were implanted (s.c.) into the right flanks of athymic nude mice. When tumor sizes reached an average size of 150-200 mm³, mice were randomized to treatment groups to start the administration of test articles or vehicle. RM-006 (also known as RMC-6272) was administered by intraperitoneal injection once weekly, and AMG 510 was administered by oral gavage daily. Body weight and tumor volume (using calipers) was measured twice weekly until study endpoints.

### Results:

Single-agent RM-006 (also known as RMC-6272) administered at 3 mg/kg IP weekly led to a TGI of 47.6%, and single-agent AMG 510 administered at 100 mg/kg PO daily led to a TGI of 71.5% in ST3235 human CRC PDX model with co-occurring KRAS^{G12C} and PIK3CA^{E545K}. However, combination of RM-006 (also known as RMC-6272) (3 mg/kg) and AMG 510 (100 mg/kg) displayed better tumor growth inhibition than either single agent group with TGI of 92.7%. The anti-tumor activity by the combination treatment was statistically significant compared with control group (***p<0.001, ordinary One-way ANOVA with multiple comparisons via a post-hoc Tukey's test).

### Example 8

Combination of RM-006 (Also Known as RMC-6272) and Compound A, a KRAS^{G12C}(ON) Inhibitor, on Tumor Growth in the Human Lung Cancer ST1989 KRAS^{G12C} Patient-Derived Xenograft Model.

### Objective:

We evaluated whether combination treatment of RM-006 (also known as RMC-6272) and Compound A, a KRAS^{G12C}(ON) inhibitor as disclosed herein, could attenuate tumor cell growth in vivo in the human lung cancer ST1989 KRAS^{G12C} patient-derived xenograft model using female athymic nude mice. Compound A is a KRAS^{G12C}(ON) inhibitor disclosed in Appendix B-1.

### Methods:

The combinatorial effects of RM-006 (also known as RMC-6272) with Compound A on tumor cell growth in vivo were evaluated in the human lung cancer ST1989 KRAS^{G12C} patient-derived xenograft model using female athymic nude mice (6-12 weeks old). Mice were implanted with tumor fragment of approximately 70 mg in size subcutaneously in the flank region. Once tumors reached an average size in the range between 150-300 mm³, mice were randomized to treatment groups with three mice per group to start the administration of test articles or vehicle. RM-006 (also known as RMC-6272) was administered by intraperitoneal injection once weekly, and Compound A was administered by oral gavage daily. Body weight and tumor volume (using calipers) was measured twice weekly until study endpoints. End of study responses in individual tumors were plotted as a waterfall plot, and the numbers indicate number of tumor regression in each group. Tumor regression is defined as greater than 10% reduction of tumor volume at the end of study relative to initial volume.

### Results:

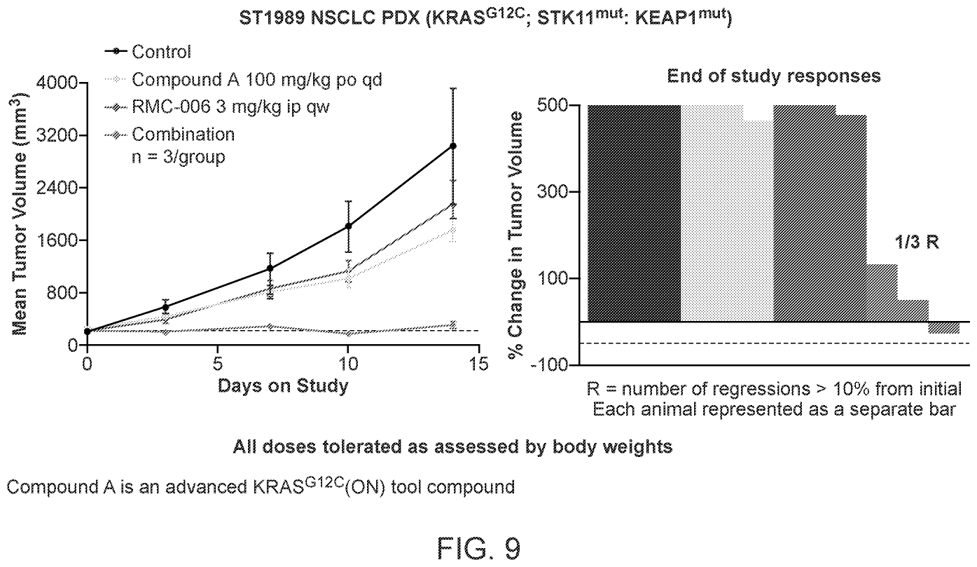

Here in FIG. 9, single-agent RM-006 (also known as RMC-6272) administered at 3 mg/kg IP weekly led to a tumor growth inhibition (TGI) of 31.6%, and single-agent Compound A administered at 100 mg/kg PO daily led to a TGI of 45.3% in ST1989 tumors. Importantly, the combination of RM-006 (also known as RMC-6272) 3 mg/kg plus Compound A 100 mg/kg led to a TGI of 96.5%. End of study responses were shown as a waterfall plot, which indicates 1 out 3 mice had tumor regression in the combination group, whereas no tumor regressions recorded in each single agent group. The combination treatment was tolerated.

### Example 9

Combinatorial Effects of RMC-6272 (Also Known as RM-006) with Compound B, a KRAS^{G12C}(ON) Inhibitor, in a NSCLC CDX Model

### Methods:

The combinatorial effects of bi-steric mTOR inhibitor RMC-6272 (also known as RM-006) with Compound B, a KRAS^{G12C}(ON) inhibitor disclosed herein, on tumor cell growth in vivo were evaluated in the human NSCLC NCI-H2122 (KRAS^{G12C}; STK11^{MUT}; KEAP1^{MUT}) cell line-derived xenograft model using female Balb/c nude mice (4-6 weeks old). Mice were implanted with NCI-H2122 cancer cells in 50% Matrigel (5×10⁶ cells/mouse) subcutaneously in the flank. Once tumors reached an average size in the range between 150-200 mm³, mice were randomized to treatment groups with eight mice per group to start the administration of test articles or vehicle. RMC-6272 (also known as RM-006) was administered by intraperitoneal (ip) injection once weekly, and Compound B was administered by oral gavage (po) daily. Body weight and tumor volume (using calipers) was measured twice weekly until study endpoints. Compound B is a KRAS^{G12C}(ON) inhibitor disclosed in Appendix B-1.

### Results:

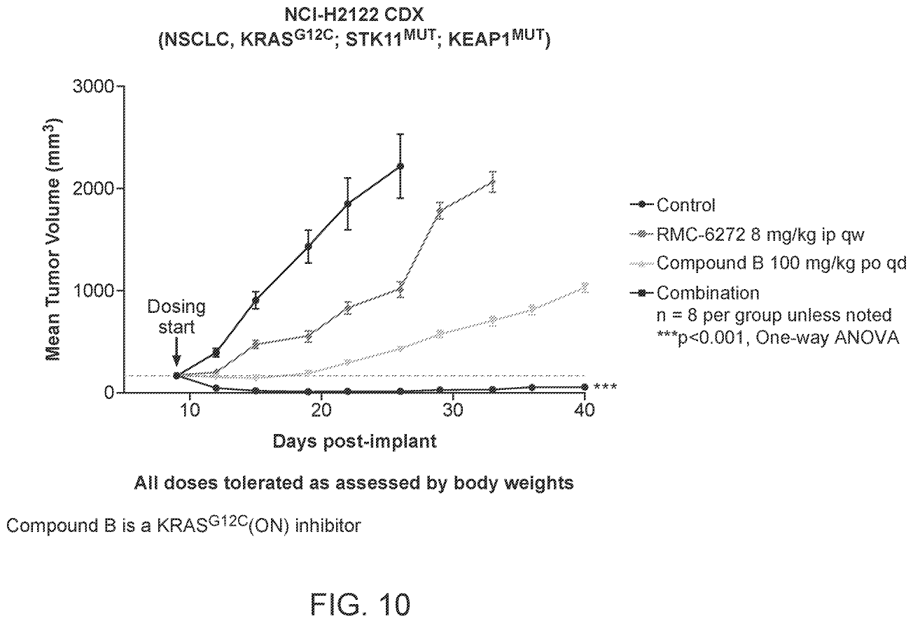

In FIG. 10, single-agent RMC-6272 (also known as RM-006 administered at 8 mg/kg ip weekly led to a tumor growth inhibition (TGI) of 59.0%, and single-agent Compound B administered at 100 mg/kg po daily led to a TGI of 87.4% at Day 17 post-dosing started in NCI-H2122 xenografted tumors. Importantly, the combination of RMC-6272 (also known as RM-006) 8 mg/kg plus Compound B 100 mg/kg led to complete regression of all tumors in the group at Day 17 post-dosing started. And all tumors in the combination group still exhibited tumor regression at Day 31 post-dosing started. All treatment was tolerated during the study course.

### Example 10

Combinatorial Effects of RMC-5552 with Compound B, a KRAS^{G12C}(ON) Inhibitor as in Example 9, in a NSCLC CDX Model

### Methods:

The combinatorial effects of bi-steric mTOR inhibitor RMC-5552 with Compound B, a KRAS^{G12C}(ON) inhibitor disclosed herein and as in Example 9, on tumor cell growth in vivo were evaluated in the human NSCLC NCI-H2122 (KRAS^{G12C}; STK11^{MUT}; KEAP1^{MUT}) cell line-derived xenograft model using female Balb/c nude mice (4-6 weeks old). Mice were implanted with NCI-H2122 cancer cells in 50% Matrigel (5×10⁶ cells/mouse) subcutaneously in the flank. Once tumors reached an average size in the range between 150-200 mm³, mice were randomized to treatment groups with eight mice per group to start the administration of test articles or vehicle. RMC-5552 was administered by intraperitoneal (ip) injection once weekly, and Compound B was administered by oral gavage (po) daily. Body weight and tumor volume (using calipers) was measured twice weekly until study endpoints. Compound B is a KRAS^{G12C}om inhibitor disclosed in Appendix B-1.

### Results:

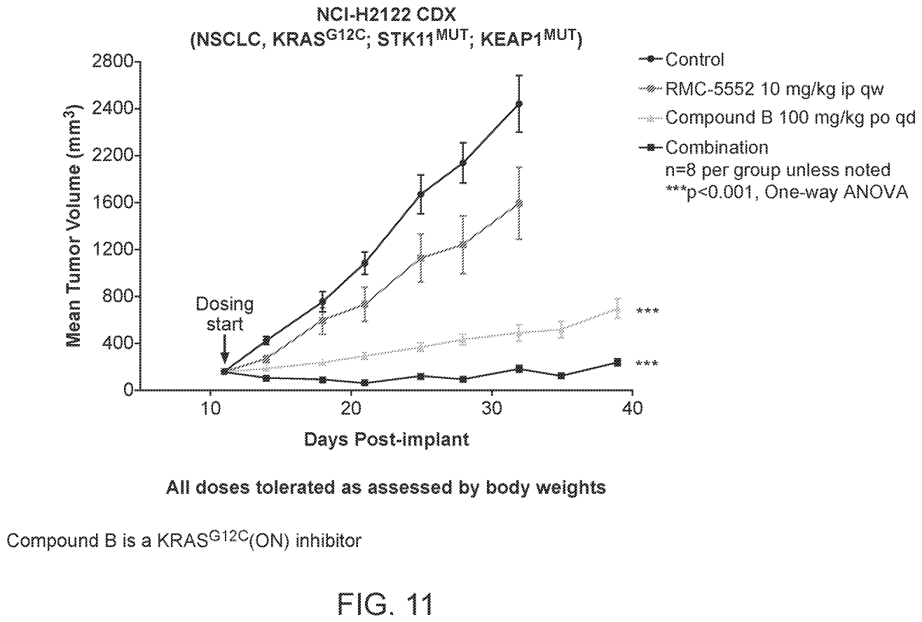

In FIG. 11, single-agent RMC-5552 administered at 10 mg/kg ip weekly led to a tumor growth inhibition (TGI) of 37.1%, and single-agent Compound B administered at 100 mg/kg po daily led to a TGI of 85.5% at Day 21 post-dosing started in NCI-H2122 xenografted tumors. Importantly, the combination of RMC-5552 10 mg/kg plus Compound B 100 mg/kg led to tumor growth inhibition (TGI) of 99.0% at Day 21 post-dosing started, with 3 out of 8 tumors exhibiting more than 10% tumor volume reduction from baseline. The anti-tumor activity of both Compound B (100 mg/kg po daily) and the combination treatment was statistically significant compared with control group (***p<0.001, ordinary One-way ANOVA with multiple comparisons via a post-hoc Tukey's test). All treatment was tolerated during the study course.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### APPENDIX A-1

### Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10%, of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### SUMMARY

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

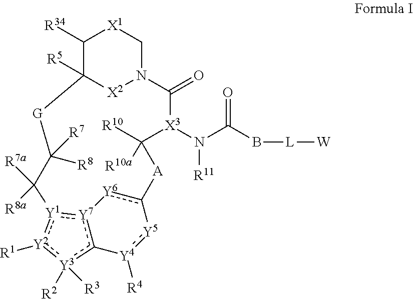

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R³⁴ is hydrogen or C₁-C₃ alkyl (e.g., methyl).

Also provided are pharmaceutical compositions comprising a compound of Formula I, or a pharmaceutically acceptable salt thereof, and a pharmaceutically acceptable excipient.

Further provided is a conjugate, or salt thereof, comprising the structure of Formula IV:

M-L-P Formula IV

wherein L is a linker;

P is a monovalent organic moiety; and

M has the structure of Formula V:

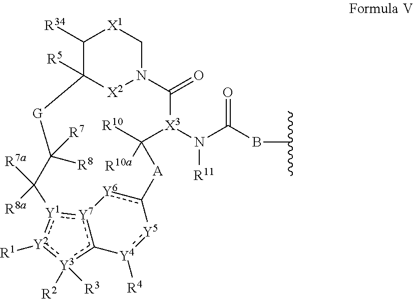

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R³⁴ is hydrogen or C₁-C₃ alkyl (e.g., methyl).

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE FIGURES

FIG. 1A: A compound of the present invention, Compound A, deeply and durably inhibits oncogenic signals in a pancreatic CDX model (HPAC CDX model, PDAC, KRAS G12D/WT). Single dose experiment, n=3/time point, all dose levels well tolerated.

FIG. 1B: Treatment of KRAS G12D tumors in vivo with a compound of the present invention, Compound A, drives tumor regressions in a pancreatic CDX model (HPAC CDX model, PDAC, KRAS G12D/WT). n=10/group, **p<0.001. All dose levels well tolerated.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4 to 8-membered saturated or unsaturated heterocycloalkyl (e.g., pyridyl); 3 to 8-membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄C(O)—N(R^{∘})—S(O)₂R^{∘}; —C(NCN)NR^{∘}₂; —(CH₂)₀₋₄C(O)SR^{∘}; —(CH₂)₀₋₄C(O)OSi R^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁-C₄ straight or branched alkylene)O—N(R^{∘})₂; or —(C₁-C₄ straight or branched alkylene)C(O)O—N(R^{∘})₂, wherein each R^{∘} may be substituted as defined below and is independently hydrogen, —C₁-C₆ aliphatic, —CH₂Ph, —O(CH₂)₀₋₁Ph, —CH₂-(5 to 6 membered heteroaryl ring), or a 3 to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3 to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —N O₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁-C₆ aliphatic which may be substituted as defined below, or an unsubstituted 5 to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁-C₆ aliphatic which may be substituted as defined below, or an unsubstituted 5 to 6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁-C₆ aliphatic which may be substituted as defined below, unsubstituted —OPh, or an unsubstituted 3 to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3 to 12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur.

Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

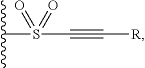

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxynorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted 3 to 12-membered monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused, or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused, or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

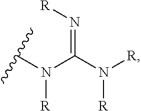

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent, monocyclic, bicyclic or polycyclic ring system, which may be bridged, fused, or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting moiety B to moiety W in a compound of Formula I, such that the resulting compound is capable of achieving an IC50 of 2 uM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

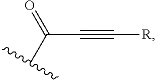

wherein R is any any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that both covalent and non-covalent interactions of a compound of the present invention with Ras and the chaperone protein (e.g., cyclophilin A) may contribute to the inhibition of Ras activity. In some embodiments, a compound of the present invention forms a covalent adduct with a side chain of a Ras protein (e.g., the —CH₂—COOH or —CH₂—COO-side chain of the aspartic acid at position 12 or 13 of a mutant Ras protein). Covalent adducts may also be formed with other side chains of Ras. In addition or alternatively, non-covalent interactions may be at play: for example, van der Waals, hydrophobic, hydrophilic, and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art. One method of determining covalent adduct formation is to perform a “cross-linking” assay, such as described in the Examples, and below:
Note—the following protocol describes a procedure for monitoring cross-linking of K-Ras G12C (GMP-PNP) to a compound of the invention. This protocol may also be executed substituting other Ras proteins or nucleotides, such as K-Ras G12D.
The purpose of this biochemical assay is to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A and 2 μM test compound, a 5 μM stock of GMP-PNP-loaded K-Ras (1-169) G12C is diluted 10-fold to yield a final concentration of 0.5 μM; with final sample volume being 100 μL.
The sample is incubated at 25° C. for a time period of up to 24 hours prior to quenching by the addition of 10 μL of 5% Formic Acid. Quenched samples are centrifuged at 15000 rpm for 15 minutes in a benchtop centrifuge before injecting a 10 μL aliquot onto a reverse phase C₄ column and eluting into the mass spectrometer with an increasing acetonitrile gradient in the mobile phase. Analysis of raw data may be carried out using Waters MassLynx MS software, with % bound calculated from the deconvoluted protein peaks for labeled and unlabeled K-Ras.

Accordingly, provided herein is a compound, or pharmaceutically acceptable salt thereof, having the structure of Formula I:

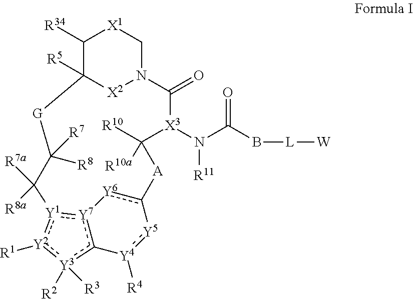

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R¹⁰ is hydrogen or halo; and

R¹¹ is hydrogen or C₁-C₃ alkyl; and

R³⁴ is hydrogen or C₁-C₃ alkyl (e.g., methyl).

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, R³⁴ is hydrogen.

In some embodiments of compounds of the present invention, G is optionally substituted C₁-C₄ heteroalkylene.

In some embodiments, a compound of the present invention has the structure of Formula Ia, or a pharmaceutically acceptable salt thereof:

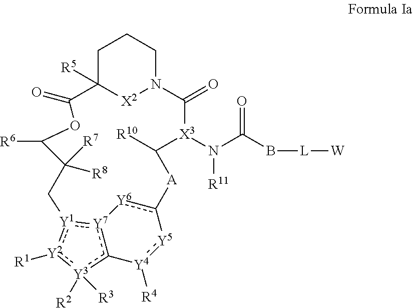

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments of compounds of the present invention, X² is NH. In some embodiments, X³ is CH.

In some embodiments of compounds of the present invention, R¹¹ is hydrogen. In some embodiments, R¹¹ is C₁-C₃ alkyl, such as methyl.

In some embodiments, a compound of the present invention has the structure of Formula Ib, or a pharmaceutically acceptable salt thereof:

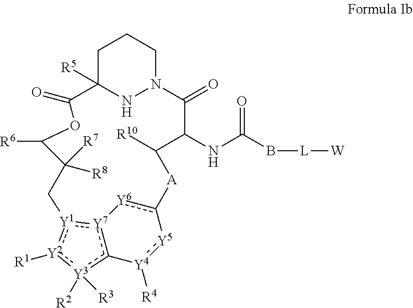

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene.

In some embodiments of compounds of the present invention, R⁴ is hydrogen.

In some embodiments of compounds of the present invention, R⁵ is hydrogen. In some embodiments, R⁵ is C₁-C₄ alkyl optionally substituted with halogen. In some embodiments, R⁵ is methyl.

In some embodiments of compounds of the present invention, Y⁴ is C. In some embodiments, R⁴ is hydrogen. In some embodiments, Y⁵ is CH. In some embodiments, Y⁶ is CH. In some embodiments, Y¹ is C. In some embodiments, Y² is C. In some embodiments, Y³ is N. In some embodiments, R³ is absent. In some embodiments, Y⁷ is C.

In some embodiments, a compound of the present invention has the structure of Formula Ic, or a pharmaceutically acceptable salt thereof:

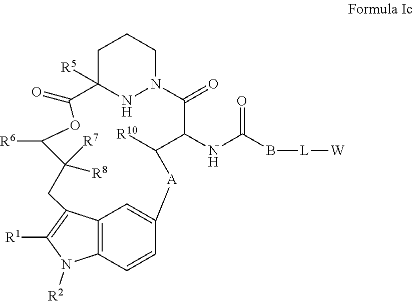

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of compounds of the present invention, R⁶ is hydrogen.

In some embodiments of compounds of the present invention, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl.

In some embodiments of compounds of the present invention, R⁷ is optionally substituted C₁-C₃ alkyl. In some embodiments, R⁷ is C₁-C₃ alkyl.

In some embodiments of compounds of the present invention, R⁸ is optionally substituted C₁-C₃ alkyl. In some embodiments, R⁸ is C₁-C₃ alkyl.

In some embodiments, a compound of the present invention has the structure of Formula Id, or a pharmaceutically acceptable salt thereof:

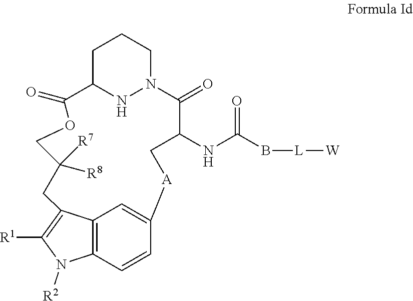

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of compounds of the present invention, R¹ is 5 to 10-membered heteroaryl.

In some embodiments, R¹ is optionally substituted 6-membered aryl or optionally substituted 6-membered heteroaryl.

In some embodiments, a compound of the present invention has the structure of Formula Ie, or a pharmaceutically acceptable salt thereof:

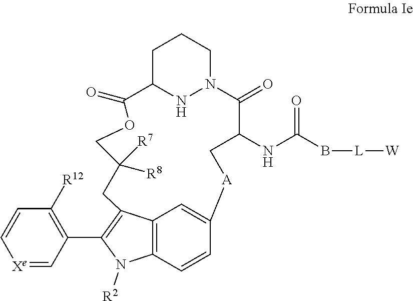

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R² is C₁-C₆ alkyl or 3 to 6-membered cycloalkyl;

R⁷ is C₁-C₃ alkyl;

R⁶ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl

X^{e} is N or CH; and

R¹² is optionally substituted C₁-C₆ alkyl or optionally substituted C₁-C₆ heteroalkyl.

In some embodiments of compounds of the present invention, X^{e} is N. In some embodiments, X^{e} is CH.

In some embodiments of compounds of the present invention, R¹² is optionally substituted C₁-C₆ heteroalkyl. In some embodiments, R¹² is

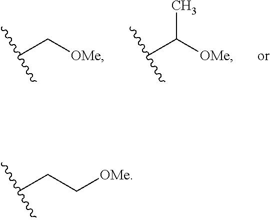

In some embodiments, R¹² is

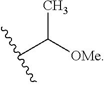 [SMILES: *C(C)OC]

In some embodiments, a compound of the present invention has the structure of Formula If, or a pharmaceutically acceptable salt thereof:

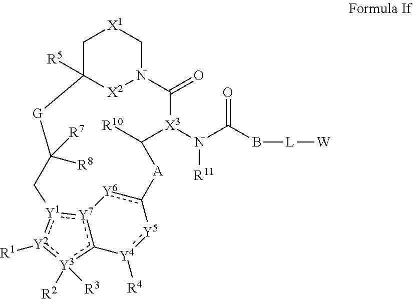

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments, a compound of the present invention has the structure of Formula VI, or a pharmaceutically acceptable salt thereof:

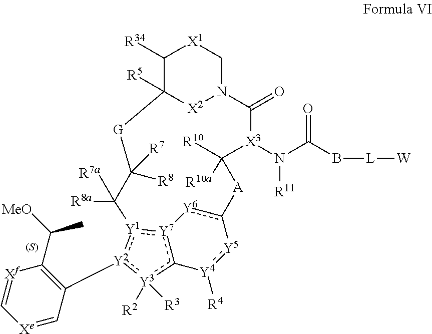

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R⁷ and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl;

R³⁴ is hydrogen or C₁-C₃ alkyl; and

X^{e} and X^{f} are, independently, N or CH.

In some embodiments, a compound of the present invention has the structure of Formula VIa, or a pharmaceutically acceptable salt thereof:

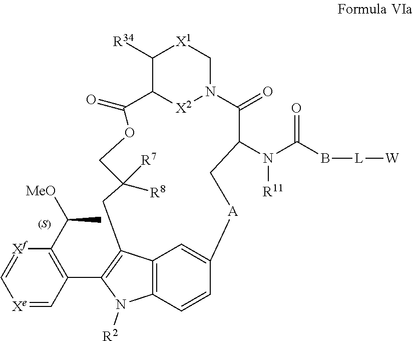

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

R² is C₁-C₆ alkyl or 3 to 6-membered cycloalkyl;

R⁷ is C₁-C₃ alkyl;

R⁶ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} and X^{f} are, independently, N or CH;

R¹¹ is hydrogen or C₁-C₃ alkyl; and

R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of Formula VIb, or a pharmaceutically acceptable salt thereof:

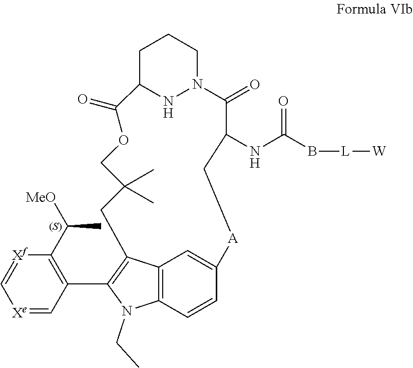

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

X^{e} and X^{f} are, independently, N or CH.

In some embodiments of a compound of the present invention, X^{e} is N and X^{f} is CH. In some embodiments, X^{e} is CH and X^{f} is N.

In some embodiments, a compound of the present invention has the structure of Formula VII, or a pharmaceutically acceptable salt thereof:

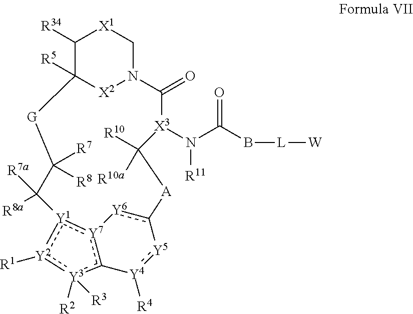

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is

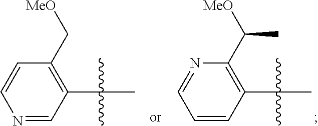 [SMILES: *c1cnccc1COC ; *c1cccnc1[C@H](C)OC]

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R³⁴ is hydrogen or C₁-C₃ alkyl (e.g., methyl).

In some embodiments of compounds of the present invention, A is optionally substituted 6-membered arylene. In some embodiments, A has the structure:

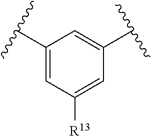

wherein R¹³ is hydrogen, hydroxy, amino, optionally substituted C₁-C₆ alkyl, or optionally substituted C₁-C₆ heteroalkyl. In some embodiments, R¹³ is hydrogen. In some embodiments, R¹³ is hydroxy.

In some embodiments of compounds of the present invention, B is —CHR⁹—. In some embodiments, R⁹ is optionally substituted C₁-C₆ alkyl or optionally substituted 3 to 6-membered cycloalkyl. In some embodiments, R⁹ is:

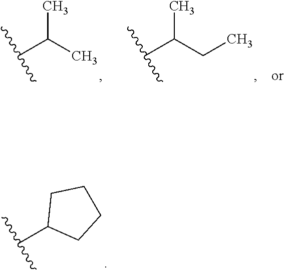

In some embodiments, R⁹ is:

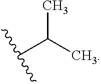 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, B is optionally substituted 6-membered arylene. In some embodiments, B is 6-membered arylene. In some embodiments, B is:

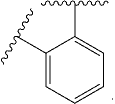 [SMILES: *c1ccccc1*]

In some embodiments of compounds of the present invention, R⁷ is methyl.

In some embodiments of compounds of the present invention, R⁸ is methyl.

In some embodiments, R³⁴ is hydrogen.

In some embodiments of compounds of the present invention, the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A². In some embodiments, the linker is acyclic. In some embodiments, the linker has the structure of Formula IIa:

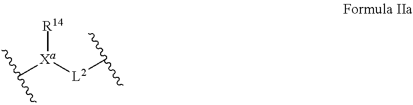

wherein X^{a} is absent or N;

R¹⁴ is absent, hydrogen or optionally substituted C₁-C₆ alkyl; and

L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

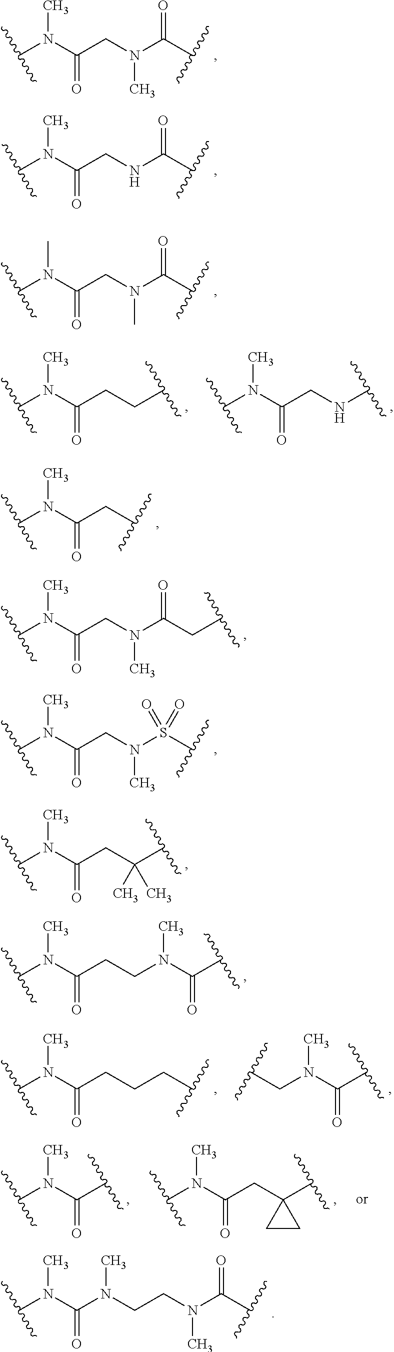

In some embodiments, the linker is or a comprises a cyclic group. In some embodiments, the linker has the structure of Formula lib:

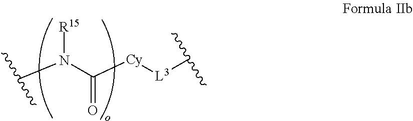

wherein o is 0 or 1;

R¹⁵ is hydrogen or optionally substituted C₁-C₆ alkyl;

Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —SO₂—, optionally substituted C₁-C₄ alkylene or optionally substituted C₁-C₄ heteroalkylene. In some embodiments, the linker has the structure:

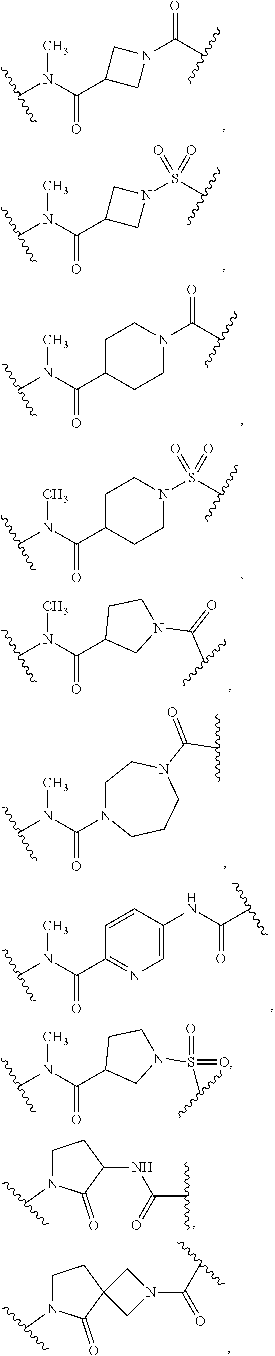

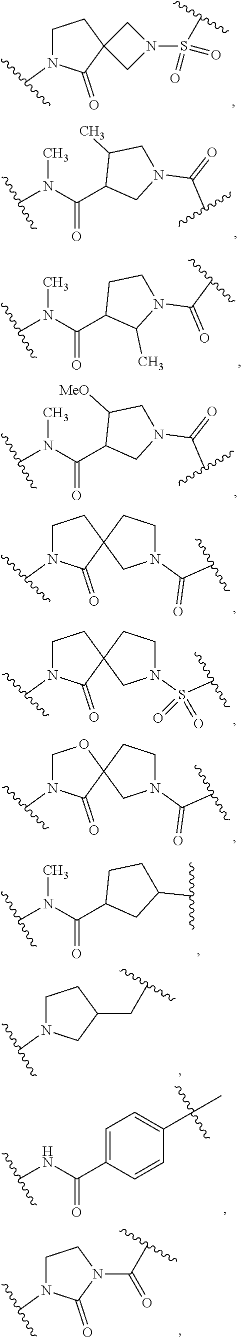

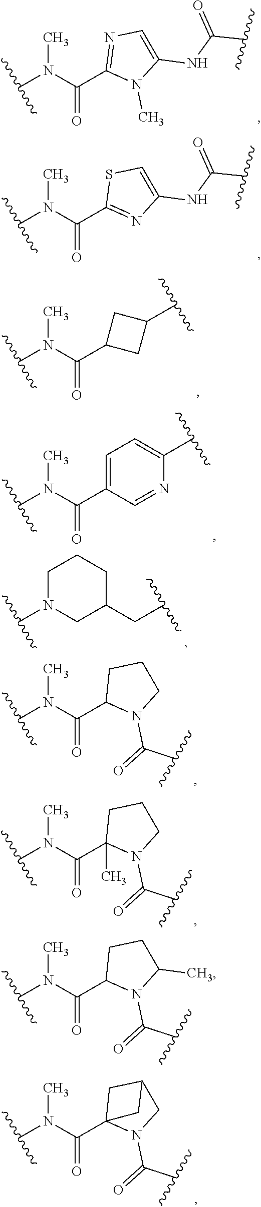

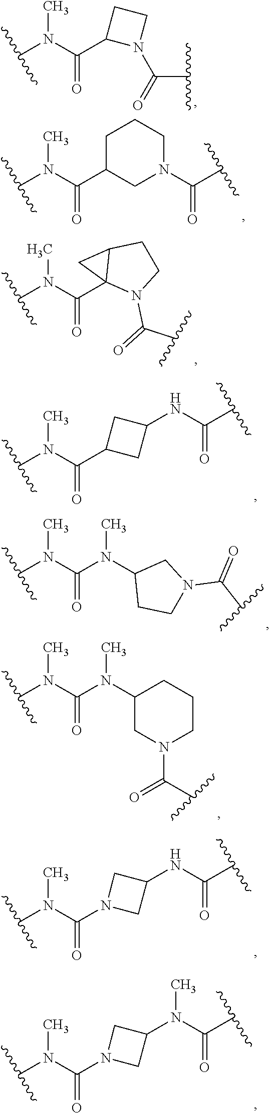

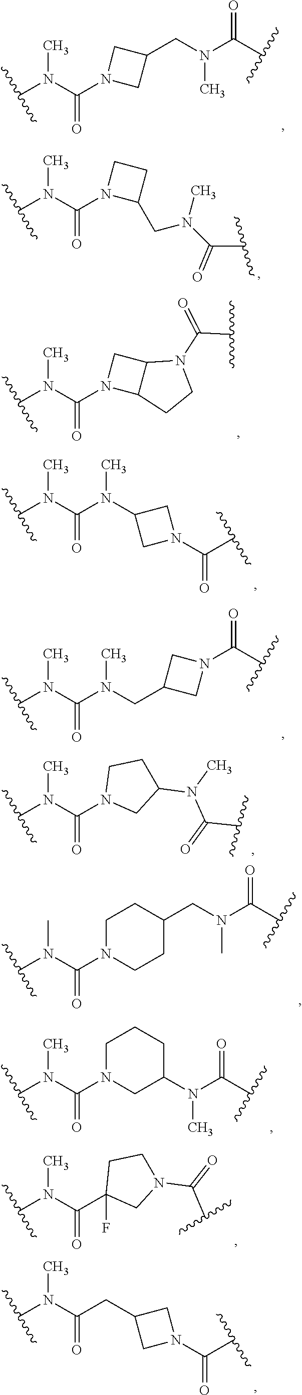

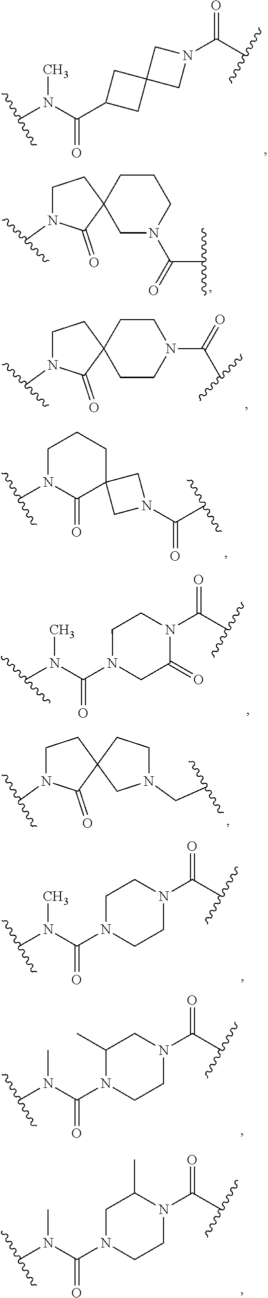

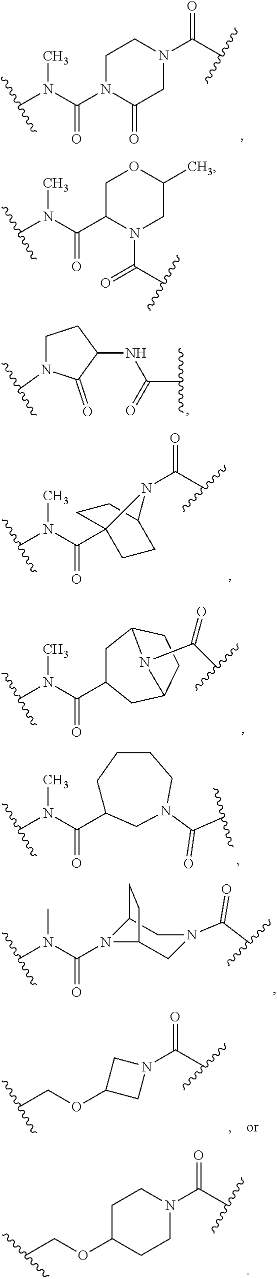

In some embodiments, a linker of Formula II is selected from the group consisting of

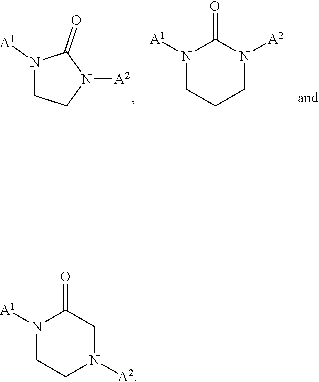

In some embodiments of compounds of the present invention, W comprises a carbodiimide. In some embodiments, W has the structure of Formula IIIa:

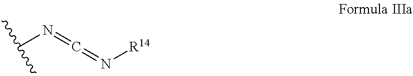

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl. In some embodiments, W has the structure:

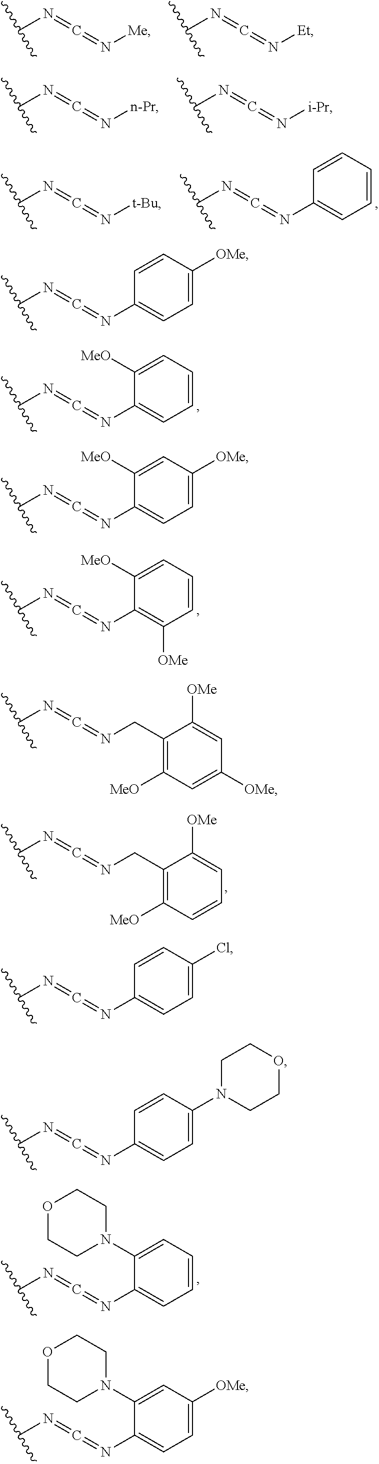

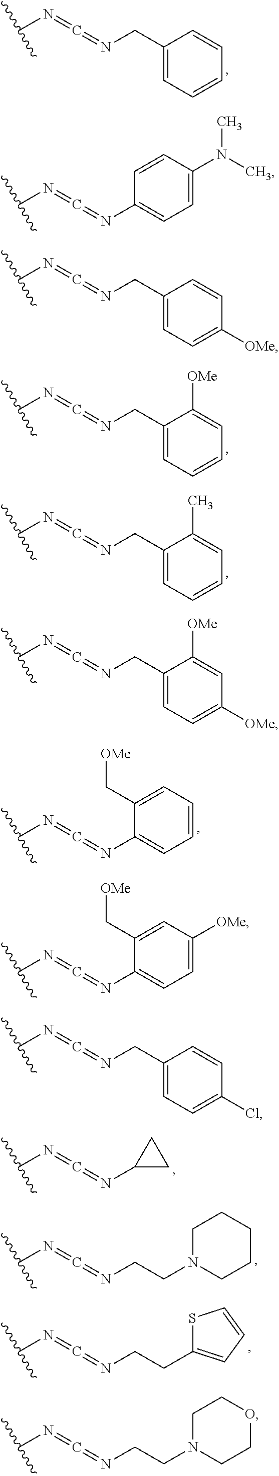

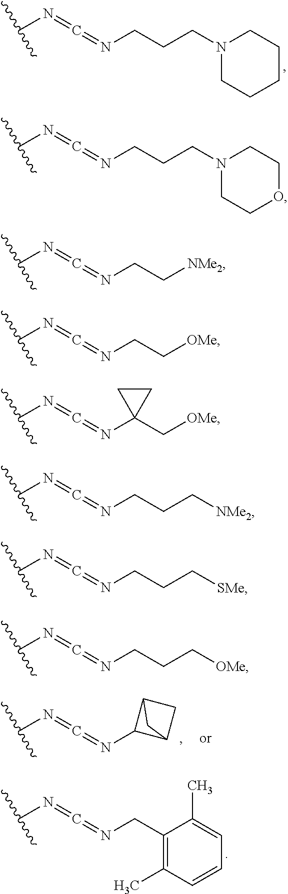

In some embodiments, W comprises an oxazoline or thiazoline. In some embodiments, W has the structure of Formula IIIb:

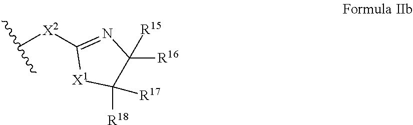

wherein X¹ is O or S;

X² is absent or NR¹⁹;

R¹⁵, R¹⁶, R¹⁷, and R¹⁸ are, independently, hydrogen or optionally substituted C₁-C₆ alkyl; and

R¹⁹ is hydrogen, C(O)(optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

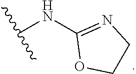 [SMILES: *NC1=NCCO1]

In some embodiments, W comprises a chloroethyl urea, a chloroethyl thiourea, a chloroethyl carbamate, or a chloroethyl thiocarbamate. In some embodiments, W has the structure of Formula IIIc:

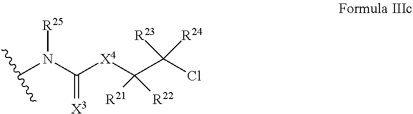

wherein X³ is O or S;

X⁴ is O, S, NR²⁶;

R²¹, R²², R²³, R²⁴, and R²⁶ are, independently, hydrogen or optionally substituted C₁-C₆ alkyl; and

R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

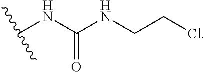 [SMILES: *NC(=O)NCCCl]

In some embodiments, W comprises an aziridine. In some embodiments, W has the structure of Formula IIId1 Formula IIId2, Formula IIId3, or Formula IIId4:

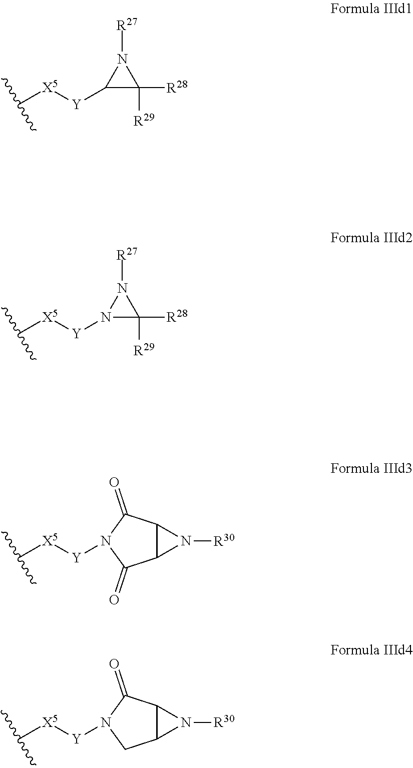

wherein X⁵ is absent or NR³⁰;

Y is absent or C(O), C(S), S(O), SO₂, or optionally substituted C₁-C₃ alkylene;

R²⁷ is hydrogen, —C(O)R³², —C(O)OR³², —SOR³³, —SO₂R³³, optionally substituted C₁-C₆ alkyl, optionally substituted 6 to 10-membered aryl, optionally substituted 3 to 14-membered heterocycloalkyl, or optionally substituted 5 to 10-membered heteroaryl;

R²⁸ and R²⁹ are, independently, hydrogen, CN, C(O)R³¹, CO₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;

each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;

R³⁰ is hydrogen or optionally substituted C₁-C₆ alkyl; and

R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is:

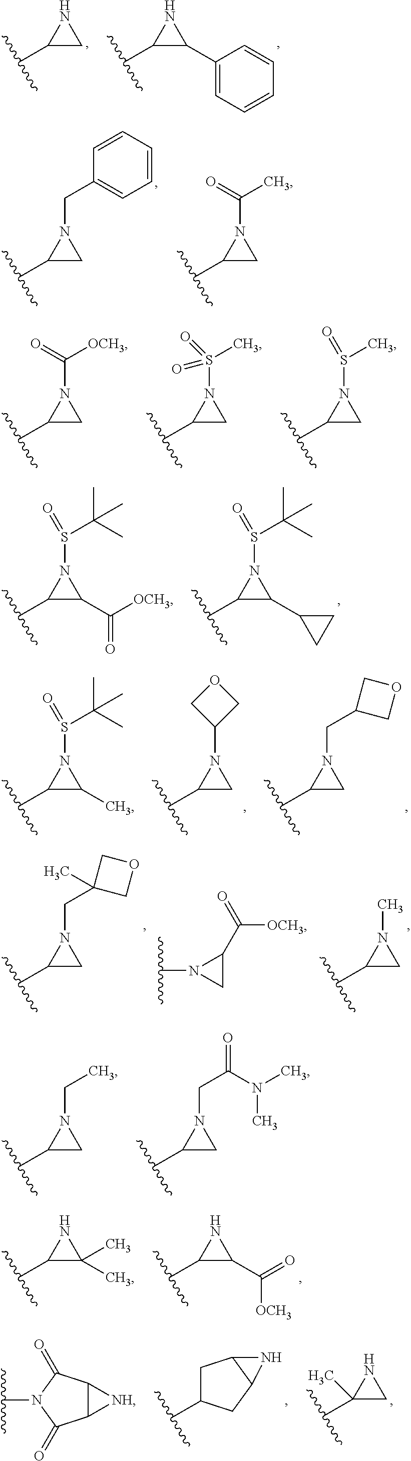

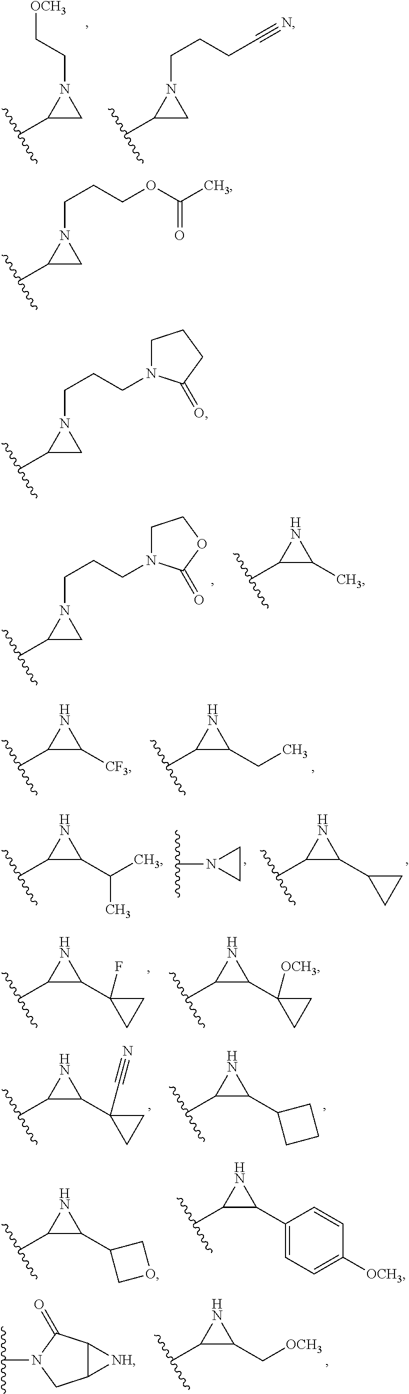

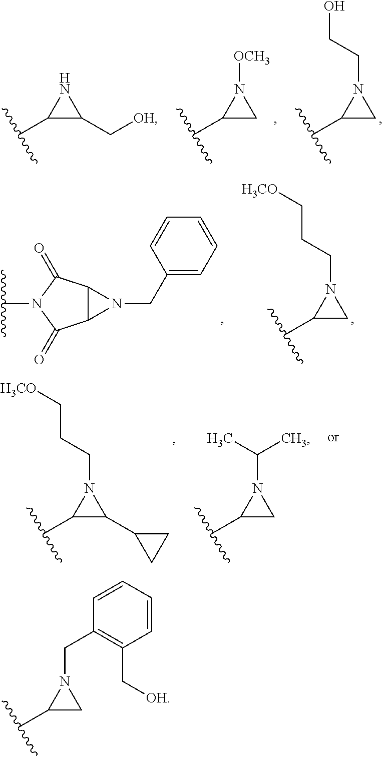

In some embodiments, W comprises an epoxide. In some embodiments, W is

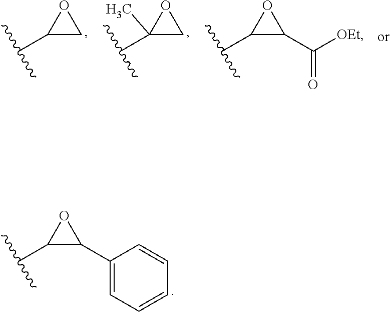

In some embodiments, W is a cross-linking group bound to an organic moiety that is a Ras binding moiety, i.e., RBM-W, wherein upon contact of an RBM-W compound with a Ras protein, the RBM-W binds to the Ras protein to form a conjugate. For example, the W moiety of an RBM-W compound may bind, e.g., cross-link, with an amino acid of the Ras protein to form the conjugate. In some embodiments, the Ras binding moiety is a K-Ras binding moiety. In some embodiments, the K-Ras binding moiety binds to a residue of a K-Ras Switch-II binding pocket of the K-Ras protein. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. The W of an RBM-W compound may comprise any W described herein. The Ras binding moiety typically has a molecular weight of under 1200 Da. See, e.g., see, e.g., Johnson et al., 292:12981-12993 (2017) for a description of Ras protein domains, incorporated herein by reference.

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present invention

| Ex# | Structure |
|---|---|
| 1 | 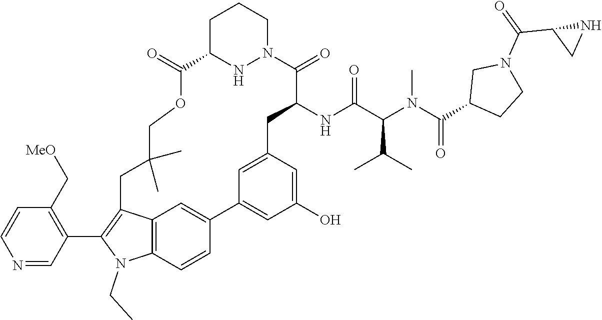 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@H]3CN3)C1)C(=O)N1CCC[C@H](N1)C(=O)OCC(C)(C)C2] |
| 2 | 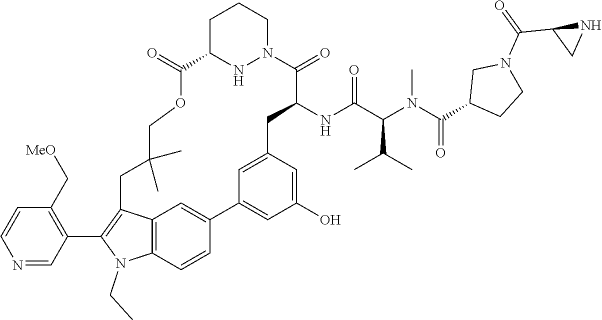 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CN3)C1)C(=O)N1CCC[C@H](N1)C(=O)OCC(C)(C)C2] |
| 3 | 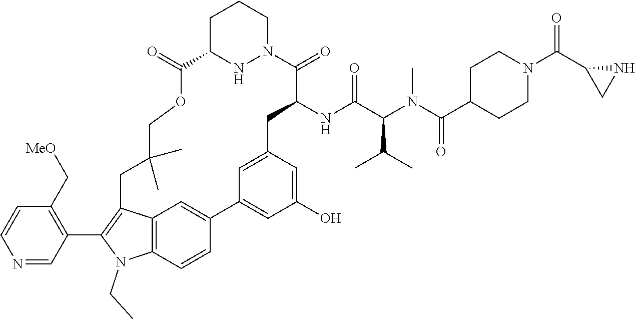 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@H]3CN3)CC1)C(=O)N1CCC[C@H](N1)C(=O)OCC(C)(C)C2] |
| 4 | 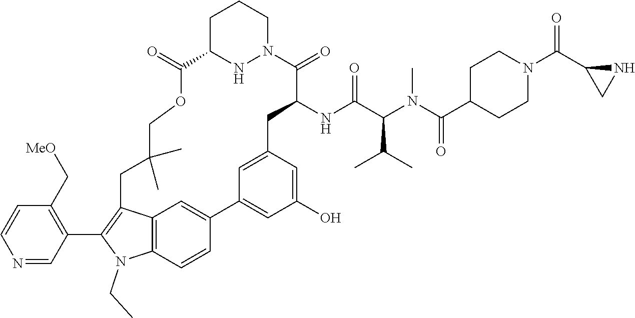 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@@H]3CN3)CC1)C(=O)N1CCC[C@H](N1)C(=O)OCC(C)(C)C2] |
| 5 | 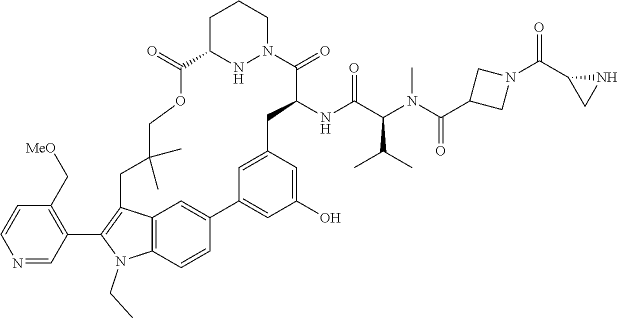 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@H]3CN3)C1)C(=O)N1CCC[C@H](N1)C(=O)OCC(C)(C)C2] |
| 6 | 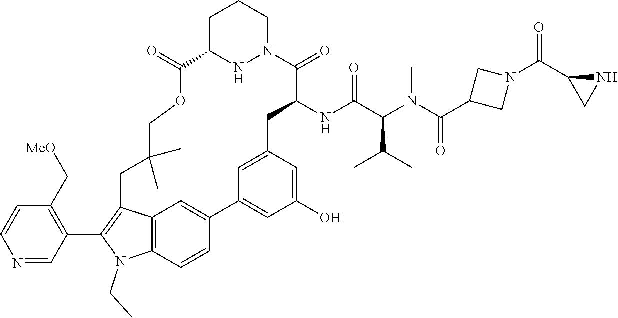 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@@H]3CN3)C1)C(=O)N1CCC[C@H](N1)C(=O)OCC(C)(C)C2] |
| 7 | 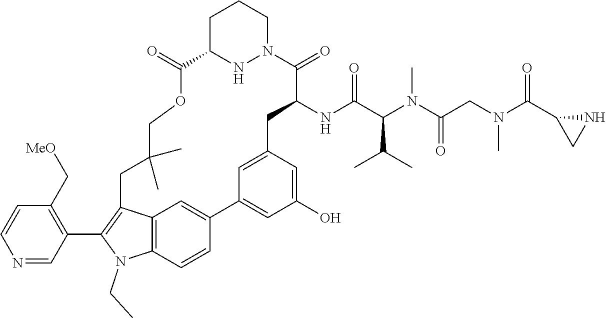 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1)C(=O)N1CCC[C@H](N1)C(=O)OCC(C)(C)C2] |
| 8 | 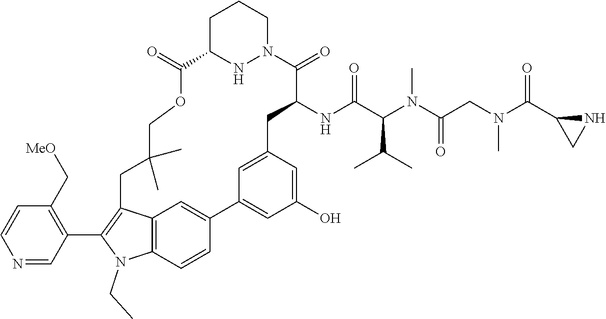 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1)C(=O)N1CCC[C@H](N1)C(=O)OCC(C)(C)C2] |
| 9 | 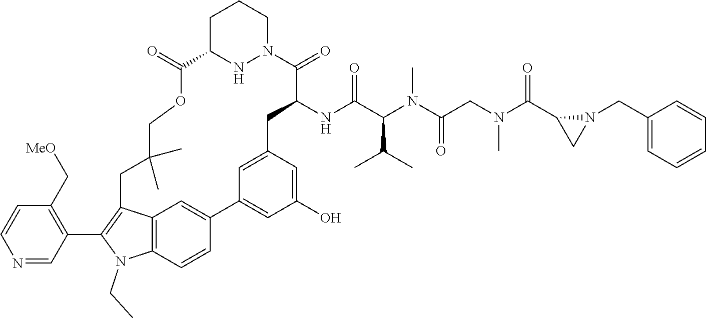 |
| 10 | 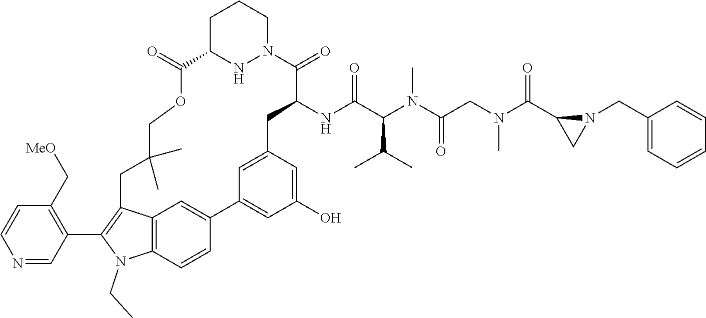 |
| 11 | 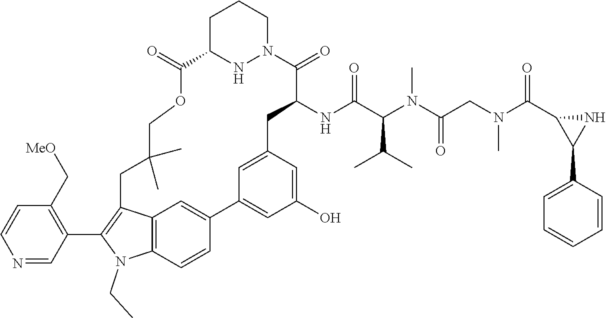 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccccc1)C(=O)N1CCC[C@H](N1)C(=O)OCC(C)(C)C2] |
| 12 | 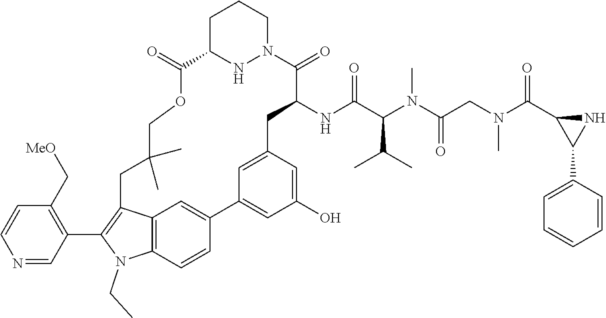 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccccc1)C(=O)N1CCC[C@H](N1)C(=O)OCC(C)(C)C2] |
| 13 | 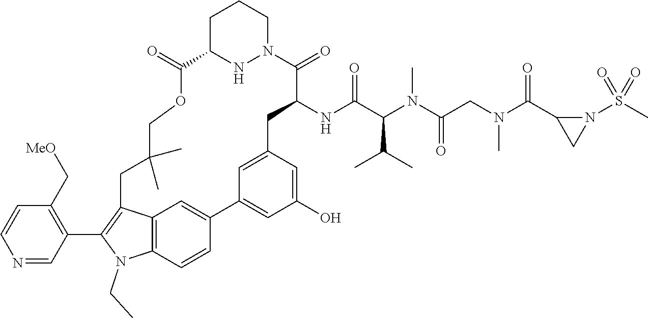 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1S(C)(=O)=O)C(=O)N1CCC[C@H](N1)C(=O)OCC(C)(C)C2] |
| 14 | 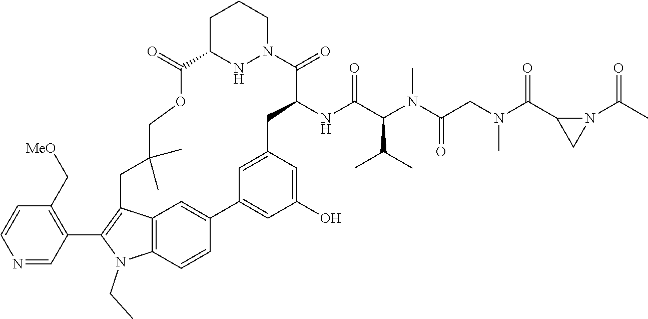 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1C(C)=O)C(=O)N1CCC[C@H](N1)C(=O)OCC(C)(C)C2] |
| 15 A and B | 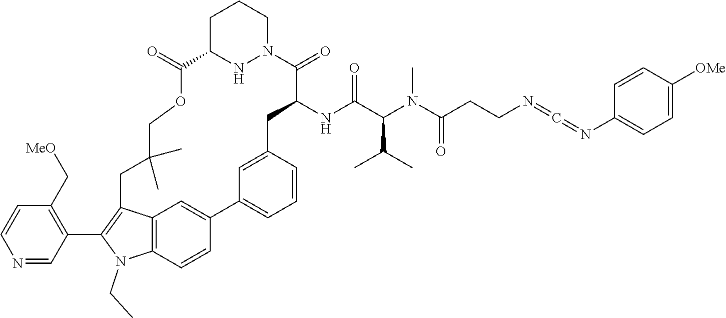 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1)C(=O)N1CCC[C@H](N1)C(=O)OCC(C)(C)C2] |
| 16 A and B | 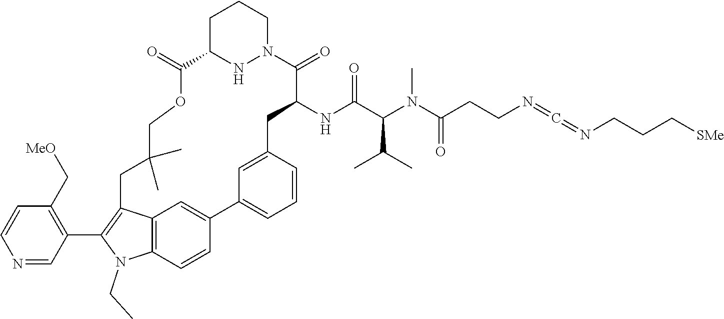 [SMILES: CCn1c(-c2cnccc2COC)c2c3cc(ccc31)-c1cccc(c1)C[C@H](NC(=O)[C@H](C(C)C)N(C)C(=O)CCN=C=NCCCSC)C(=O)N1CCC[C@H](N1)C(=O)OCC(C)(C)C2] |
| 17 A and B | 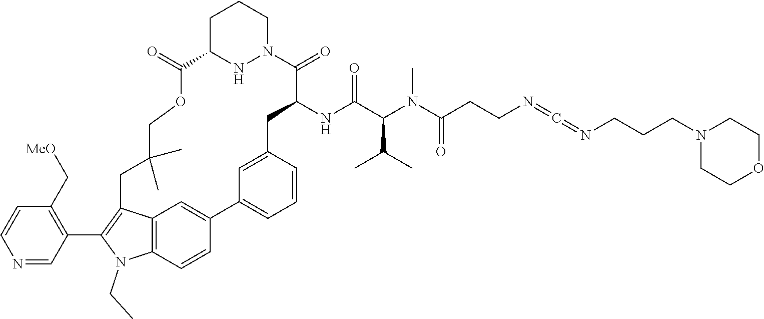 [SMILES: CCn1c(-c2cnccc2COC)c2c3cc(ccc31)-c1cccc(c1)C[C@H](NC(=O)[C@H](C(C)C)N(C)C(=O)CCN=C=NCCCN1CCOCC1)C(=O)N1CCC[C@H](N1)C(=O)OCC(C)(C)C2] |
| 18 A and B | 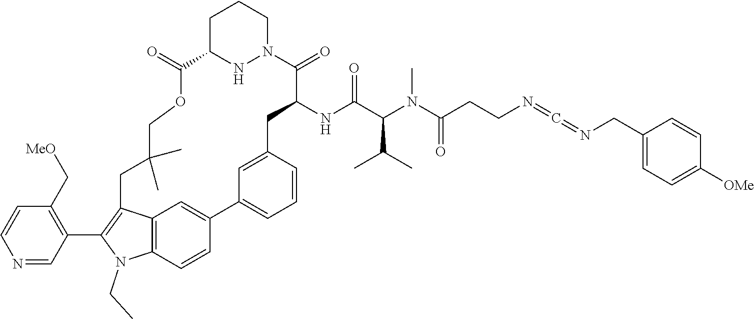 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1)C(=O)N1CCC[C@H](N1)C(=O)OCC(C)(C)C2] |
| 19 A and B | 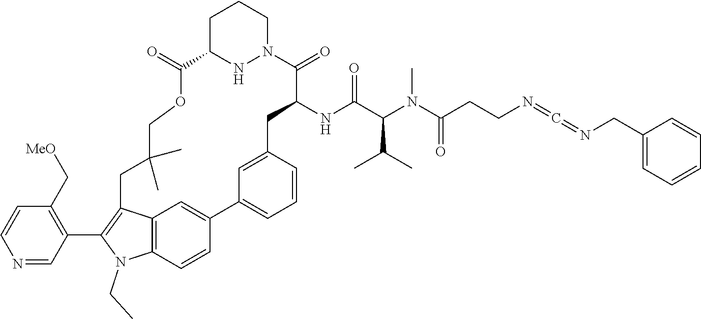 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1)C(=O)N1CCC[C@H](N1)C(=O)OCC(C)(C)C2] |
| 20 A and B | 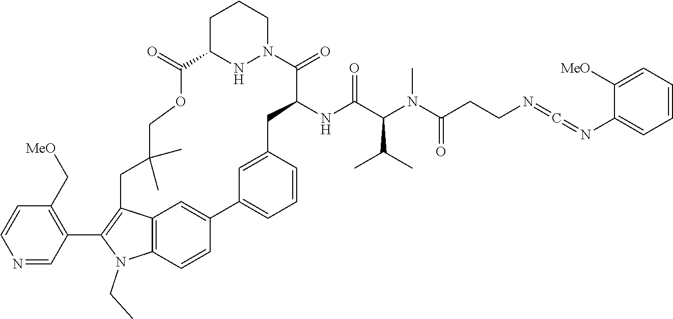 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1OC)C(=O)N1CCC[C@H](N1)C(=O)OCC(C)(C)C2] |
| 21 A and B | 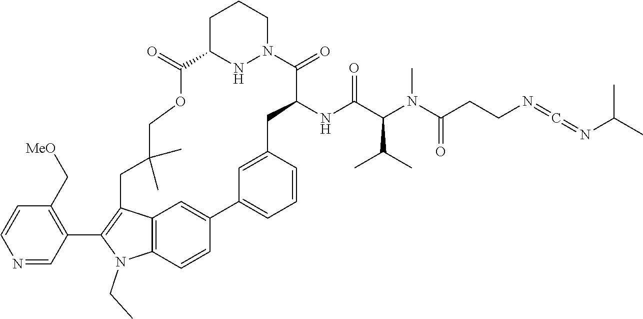 [SMILES: CCn1c(-c2cnccc2COC)c2c3cc(ccc31)-c1cccc(c1)C[C@H](NC(=O)[C@H](C(C)C)N(C)C(=O)CCN=C=NC(C)C)C(=O)N1CCC[C@H](N1)C(=O)OCC(C)(C)C2] |
| 22 A and B | 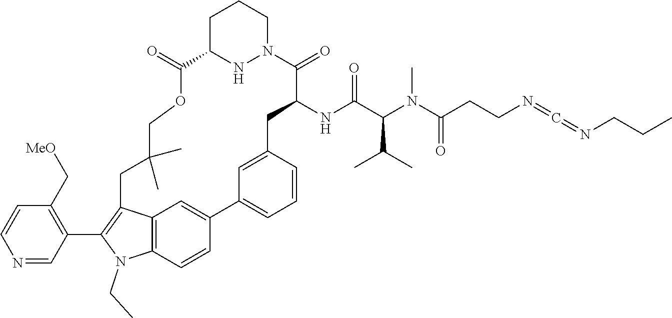 [SMILES: CCCN=C=NCCC(=O)N(C)[C@H](C(=O)N[C@H]1Cc2cccc(c2)-c2ccc3c(c2)c(c(-c2cnccc2COC)n3CC)CC(C)(C)COC(=O)[C@@H]2CCCN(N2)C1=O)C(C)C] |
| 23 A and B | 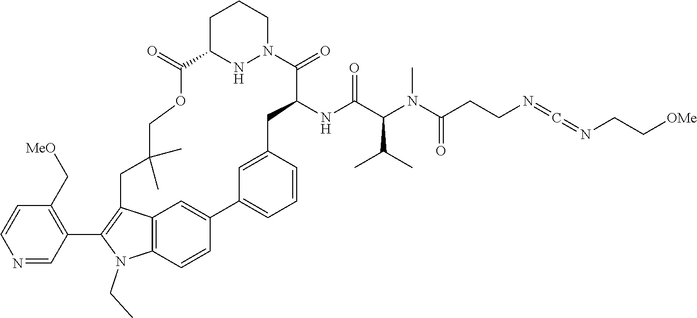 [SMILES: CCn1c(-c2cnccc2COC)c2c3cc(ccc31)-c1cccc(c1)C[C@H](NC(=O)[C@H](C(C)C)N(C)C(=O)CCN=C=NCCOC)C(=O)N1CCC[C@H](N1)C(=O)OCC(C)(C)C2] |
| 24 A and B | 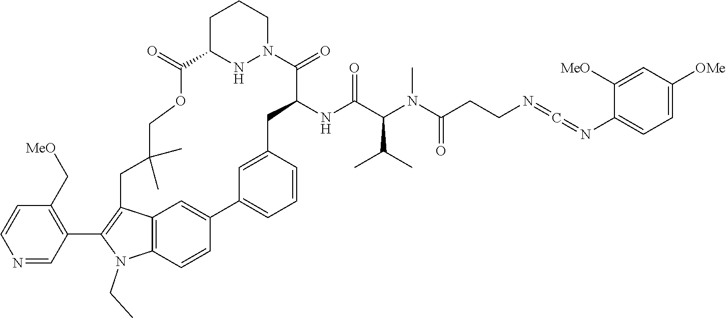 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1OC)C(=O)N1CCC[C@H](N1)C(=O)OCC(C)(C)C2] |
| 25 | 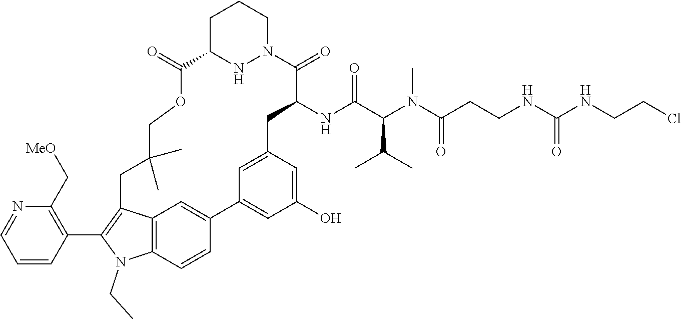 [SMILES: CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2] |
| 26 | 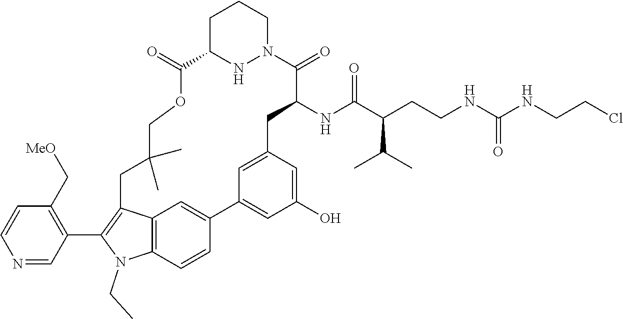 [SMILES: CCn1c(-c2cnccc2COC)c2c3cc(ccc31)-c1cc(O)cc(c1)C[C@H](NC(=O)[C@@H](CCNC(=O)NCCCl)C(C)C)C(=O)N1CCC[C@H](N1)C(=O)OCC(C)(C)C2] |
| 27 | 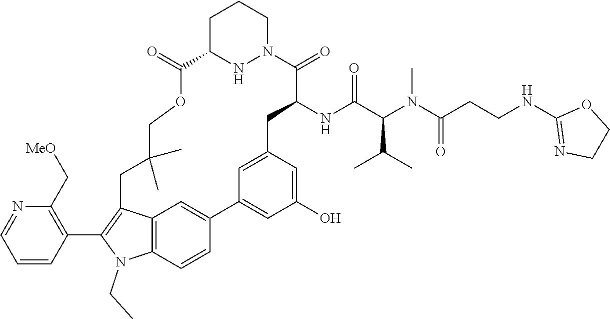 [SMILES: CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| 28 | 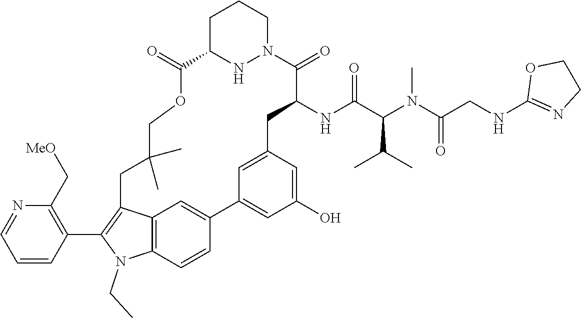 [SMILES: CCn1c(-c2cccnc2COC)c2c3cc(ccc31)-c1cc(O)cc(c1)C[C@H](NC(=O)[C@H](C(C)C)N(C)C(=O)CNC1=NCCO1)C(=O)N1CCC[C@H](N1)C(=O)OCC(C)(C)C2] |
| 29 | 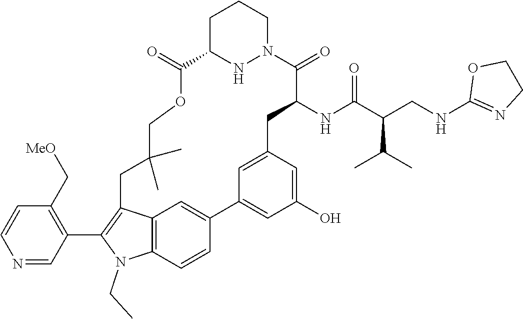 [SMILES: CCn1c(-c2cnccc2COC)c2c3cc(ccc31)-c1cc(O)cc(c1)C[C@H](NC(=O)[C@@H](CNC1=NCCO1)C(C)C)C(=O)N1CCC[C@H](N1)C(=O)OCC(C)(C)C2] |
| 30 | 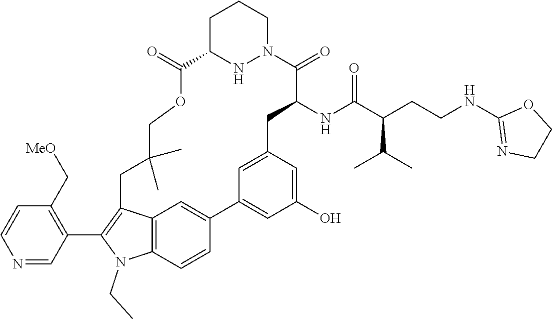 [SMILES: CCn1c(-c2cnccc2COC)c2c3cc(ccc31)-c1cc(O)cc(c1)C[C@H](NC(=O)[C@@H](CCNC1=NCCO1)C(C)C)C(=O)N1CCC[C@H](N1)C(=O)OCC(C)(C)C2] |
| 31 | 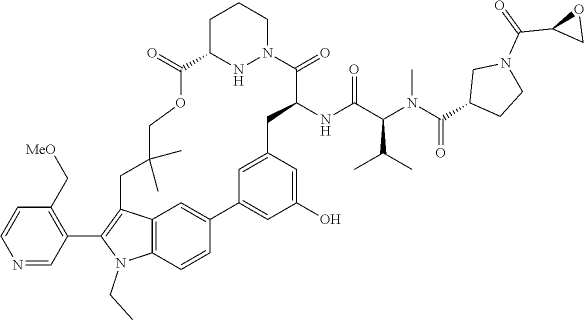 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CO3)C1)C(=O)N1CCC[C@H](N1)C(=O)OCC(C)(C)C2] |
| 32 A and B | 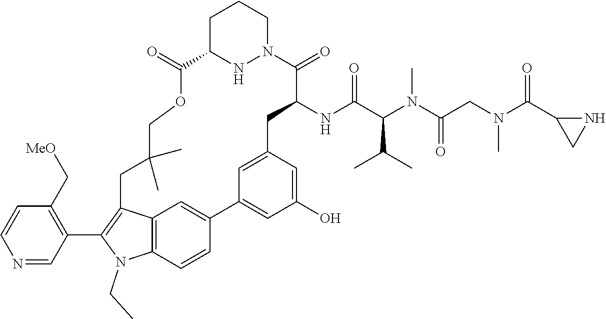 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| 33 | 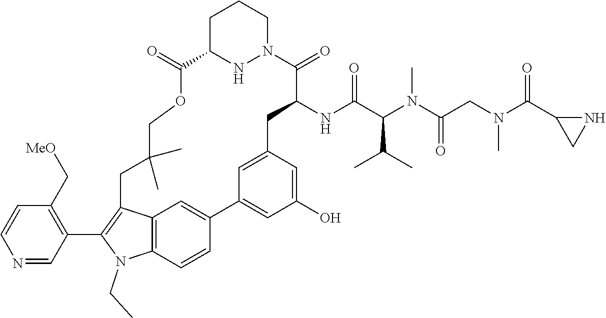 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| 34 | 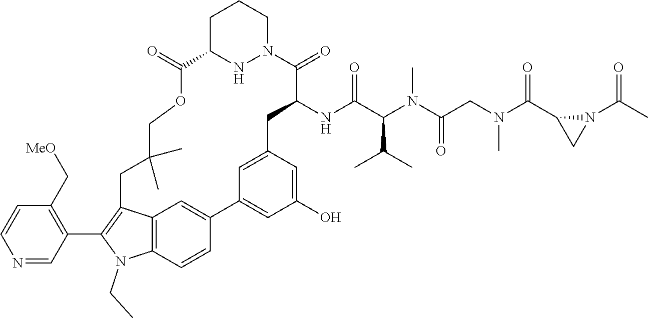 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(C)=O)C(=O)N1CCC[C@H](N1)C(=O)OCC(C)(C)C2] |
| 35 | 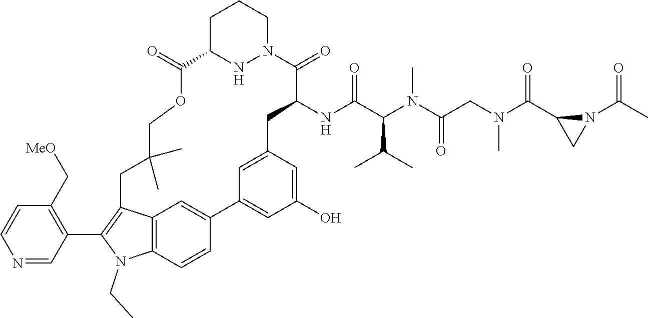 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(C)=O)C(=O)N1CCC[C@H](N1)C(=O)OCC(C)(C)C2] |
| 36 | 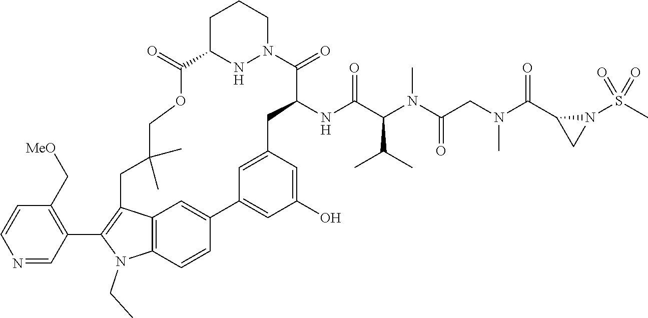 |
| 37 | 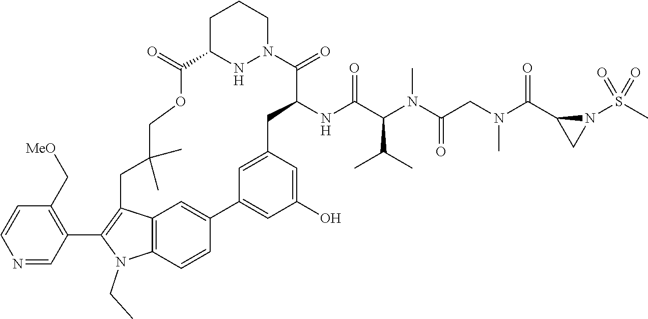 |
| 38 | 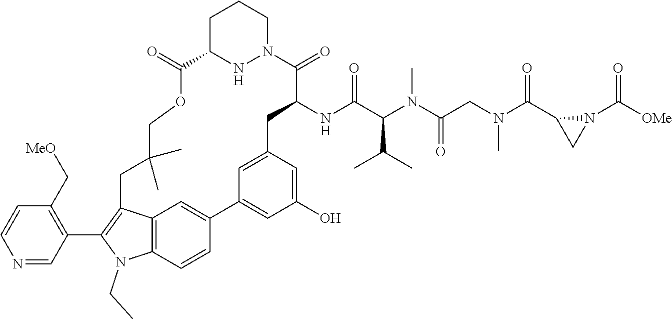 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(=O)OC)C(=O)N1CCC[C@H](N1)C(=O)OCC(C)(C)C2] |
| 39 | 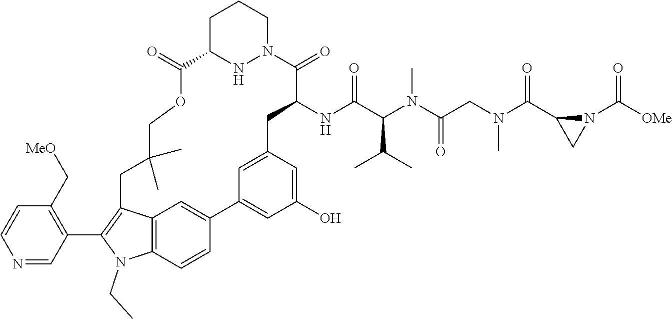 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(=O)OC)C(=O)N1CCC[C@H](N1)C(=O)OCC(C)(C)C2] |
| 40 | 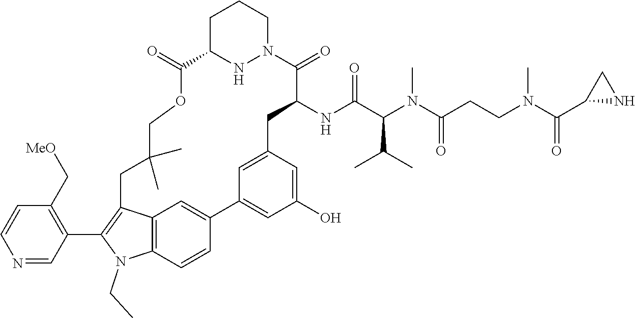 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@@H]1CN1)C(=O)N1CCC[C@H](N1)C(=O)OCC(C)(C)C2] |
| 41 | 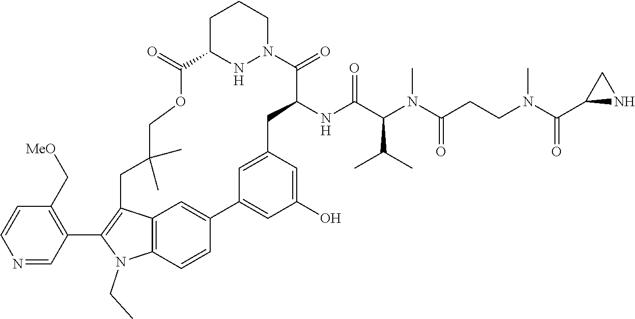 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@H]1CN1)C(=O)N1CCC[C@H](N1)C(=O)OCC(C)(C)C2] |
| 42A | 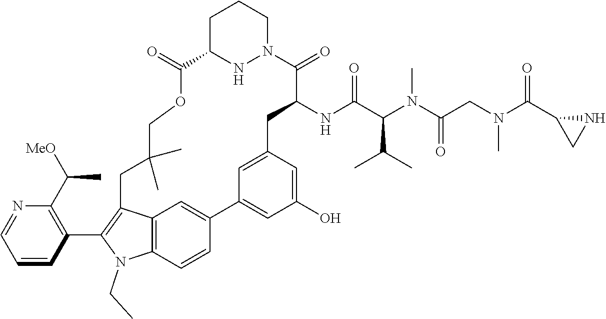 |
| 42B | 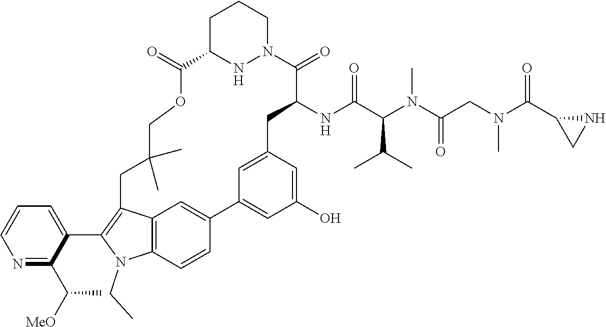 |
| 43A | 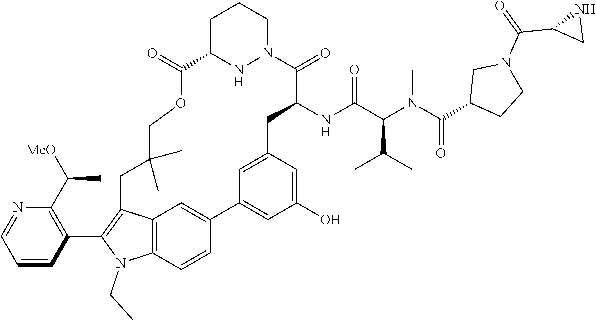 |
| 43B | 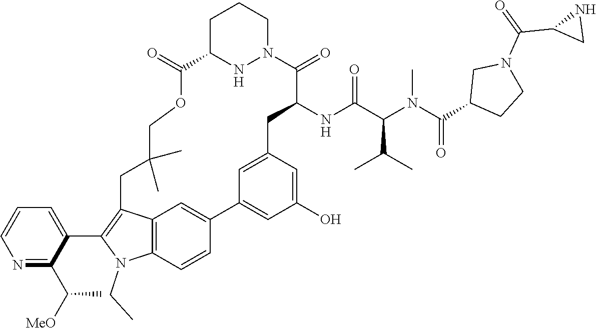 |
| 44 | 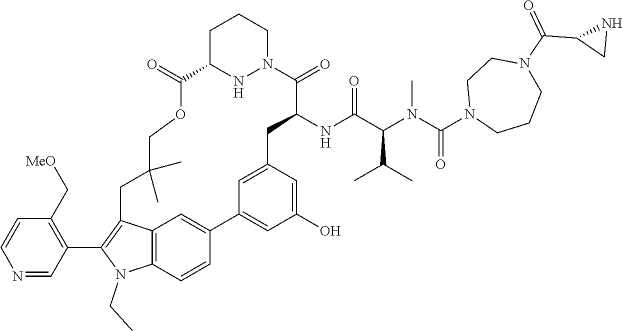 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@H]3CN3)CC1)C(=O)N1CCC[C@H](N1)C(=O)OCC(C)(C)C2] |
| 45 | 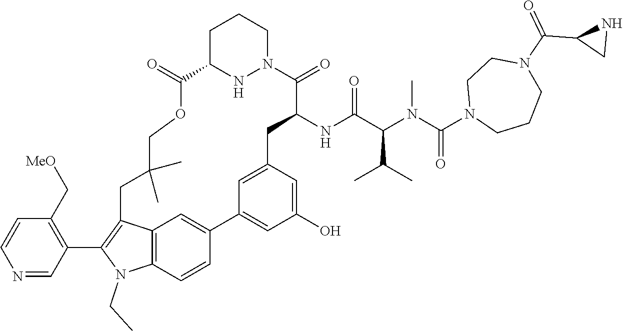 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@@H]3CN3)CC1)C(=O)N1CCC[C@H](N1)C(=O)OCC(C)(C)C2] |
| 46 | 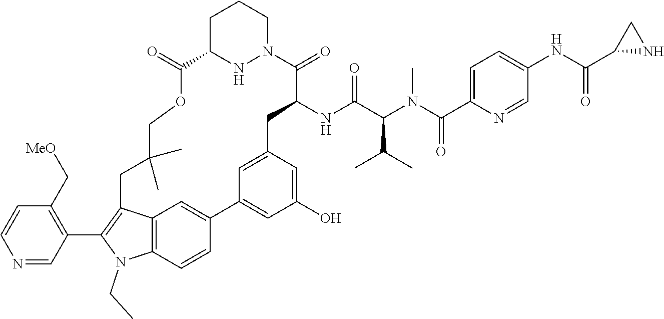 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3CN3)cn1)C(=O)N1CCC[C@H](N1)C(=O)OCC(C)(C)C2] |
| 47 A and B | 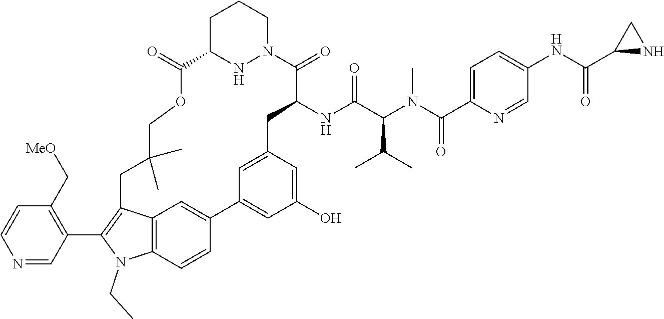 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3CN3)cn1)C(=O)N1CCC[C@H](N1)C(=O)OCC(C)(C)C2] |
| 48 | 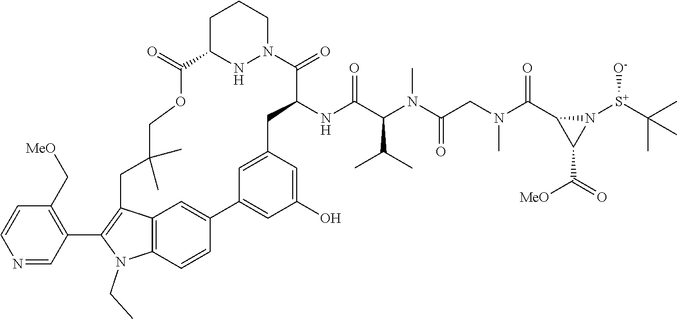 |
| 49 | 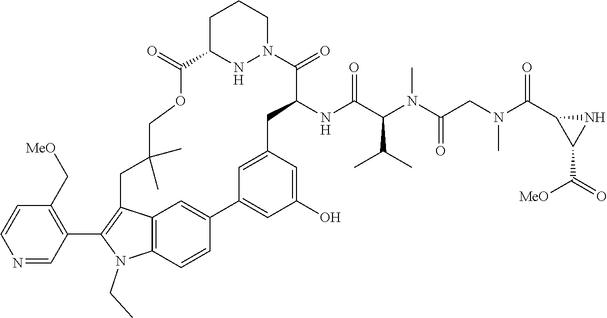 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C(=O)OC)C(=O)N1CCC[C@H](N1)C(=O)OCC(C)(C)C2] |
| 50 | 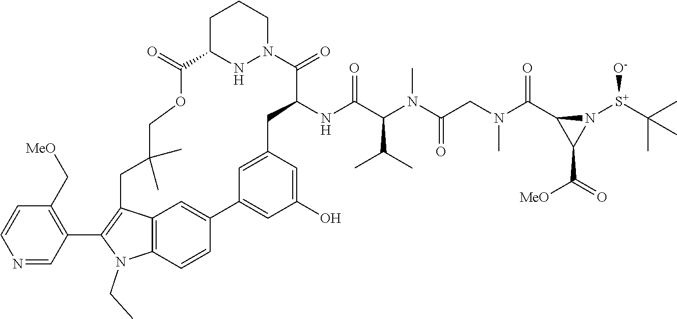 |
| 51 | 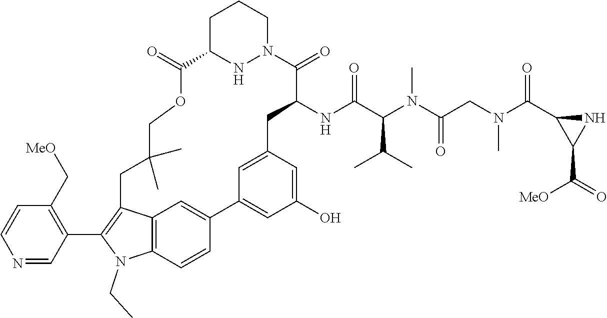 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C(=O)OC)C(=O)N1CCC[C@H](N1)C(=O)OCC(C)(C)C2] |
| 52 | 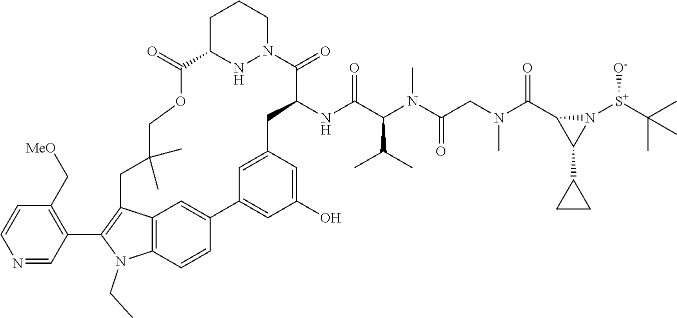 |
| 53 | 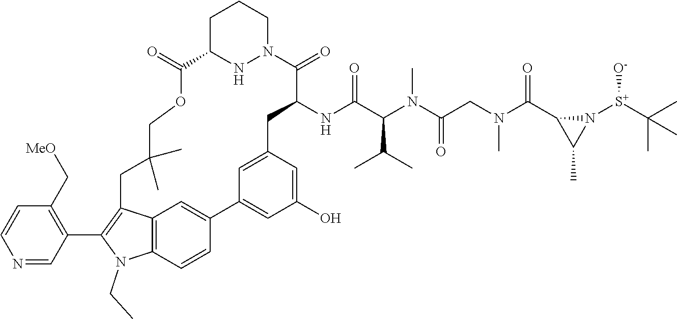 |
| 54 | 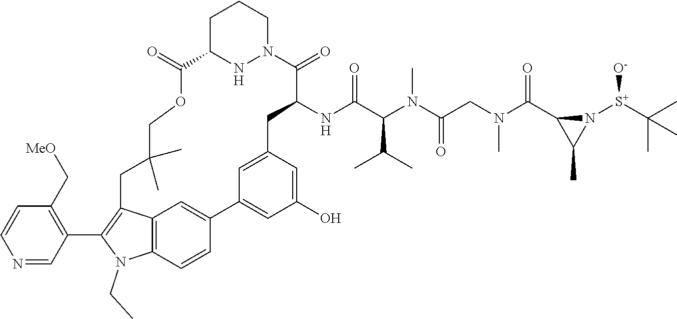 |
| 55 | 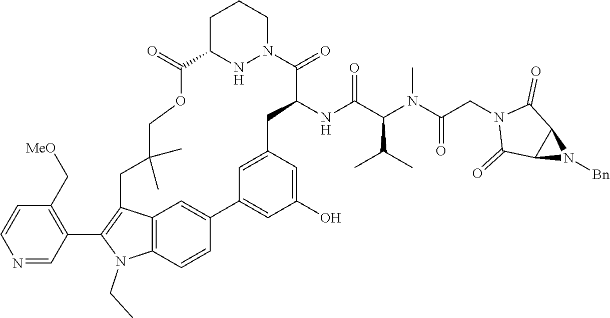 |
| 56* | 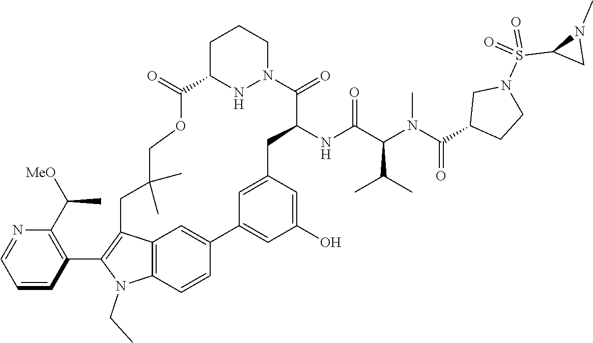 |
| 57* | 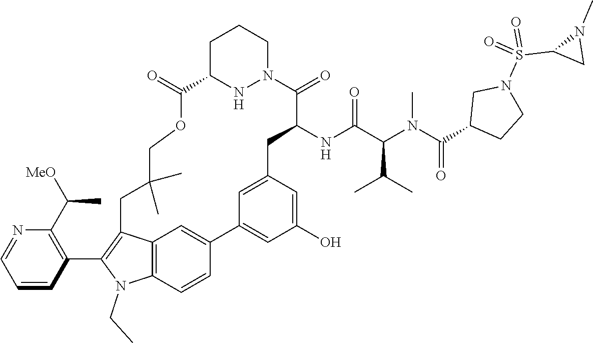 |
| 58 | 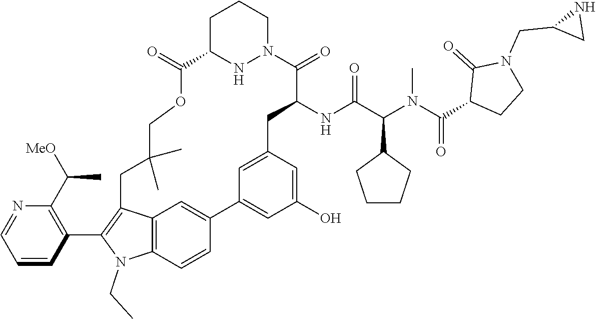 |
| 59 | 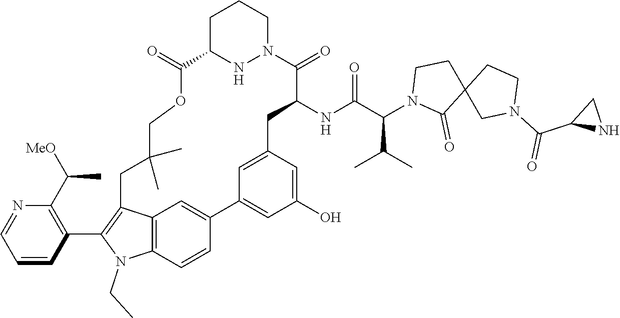 |
| 60 | 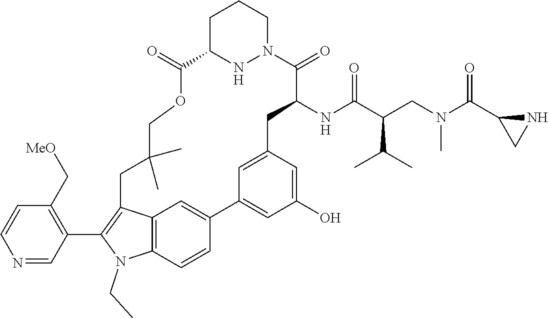 [SMILES: CCn1c(-c2cnccc2COC)c2c3cc(ccc31)-c1cc(O)cc(c1)C[C@H](NC(=O)[C@@H](CN(C)C(=O)[C@@H]1CN1)C(C)C)C(=O)N1CCC[C@H](N1)C(=O)OCC(C)(C)C2] |
| 61 A and B | 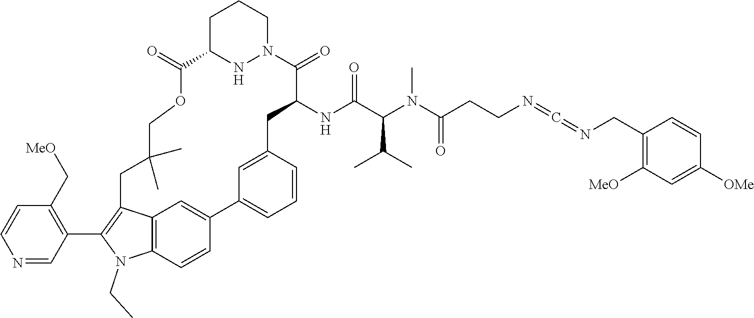 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1OC)C(=O)N1CCC[C@H](N1)C(=O)OCC(C)(C)C2] |
| 62 A and B | 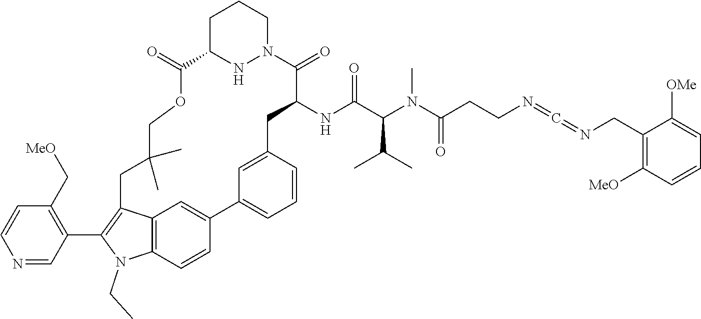 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| 63 | 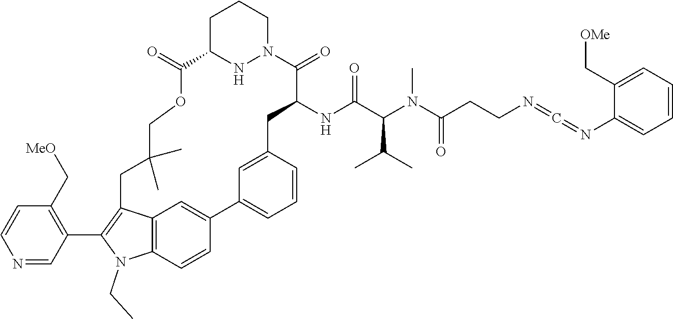 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1COC)C(=O)N1CCC[C@H](N1)C(=O)OCC(C)(C)C2] |
| 64 A and B | 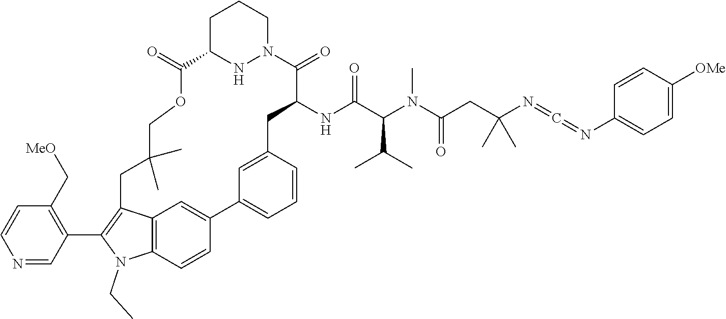 [SMILES: CCn1c(-c2cnccc2COC)c2c3cc(ccc31)-c1cccc(c1)C[C@H](NC(=O)[C@H](C(C)C)N(C)C(=O)CC(C)(C)N=C=Nc1ccc(OC)cc1)C(=O)N1CCC[C@H](N1)C(=O)OCC(C)(C)C2] |
| 65 A and B | 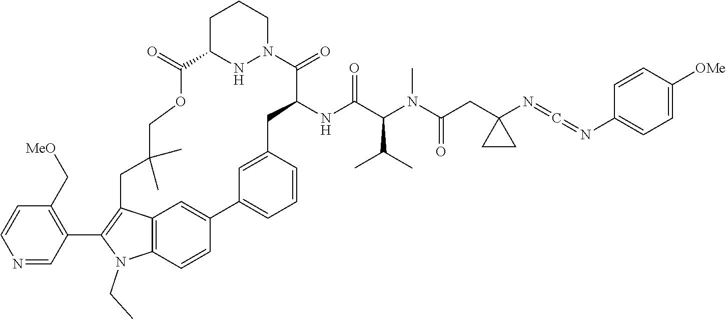 [SMILES: CCn1c(-c2cnccc2COC)c2c3cc(ccc31)-c1cccc(c1)C[C@H](NC(=O)[C@H](C(C)C)N(C)C(=O)CC1(N=C=Nc3ccc(OC)cc3)CC1)C(=O)N1CCC[C@H](N1)C(=O)OCC(C)(C)C2] |
| 66 | 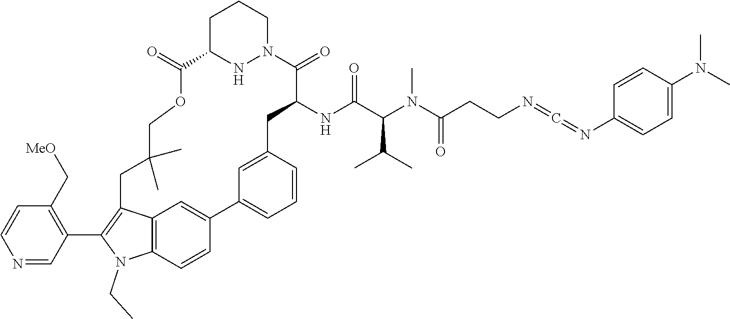 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N(C)C)cc1)C(=O)N1CCC[C@H](N1)C(=O)OCC(C)(C)C2] |
| 67 | 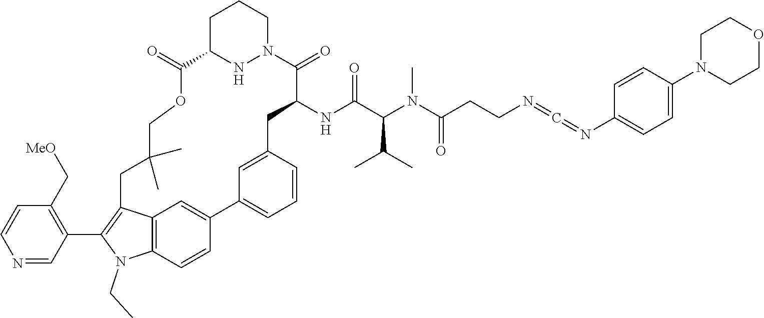 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N3CCOCC3)cc1)C(=O)N1CCC[C@H](N1)C(=O)OCC(C)(C)C2] |
| 68 A and B | 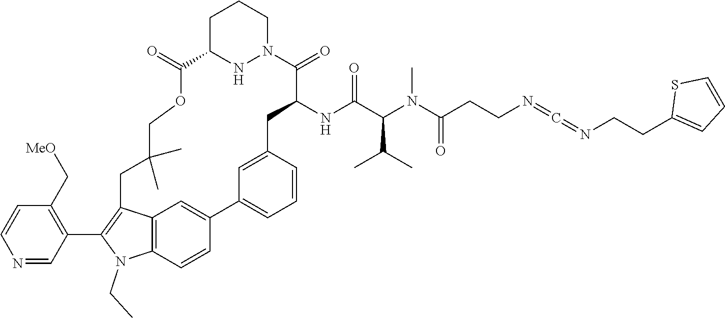 [SMILES: CCn1c(-c2cnccc2COC)c2c3cc(ccc31)-c1cccc(c1)C[C@H](NC(=O)[C@H](C(C)C)N(C)C(=O)CCN=C=NCCc1cccs1)C(=O)N1CCC[C@H](N1)C(=O)OCC(C)(C)C2] |
| 69 A and B | 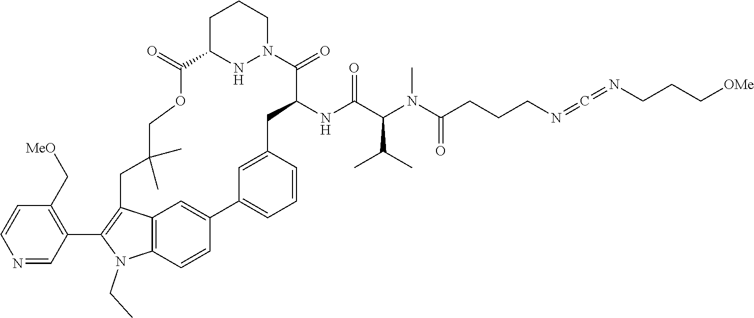 [SMILES: CCn1c(-c2cnccc2COC)c2c3cc(ccc31)-c1cccc(c1)C[C@H](NC(=O)[C@H](C(C)C)N(C)C(=O)CCCN=C=NCCCOC)C(=O)N1CCC[C@H](N1)C(=O)OCC(C)(C)C2] |
| 70 A and B | 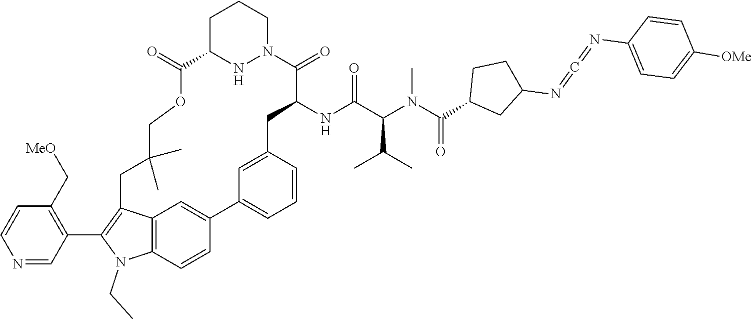 [SMILES: CCn1c(-c2cnccc2COC)c2c3cc(ccc31)-c1cccc(c1)C[C@H](NC(=O)[C@H](C(C)C)N(C)C(=O)[C@@H]1CCC(N=C=Nc3ccc(OC)cc3)C1)C(=O)N1CCC[C@H](N1)C(=O)OCC(C)(C)C2] |
| 71 A and B | 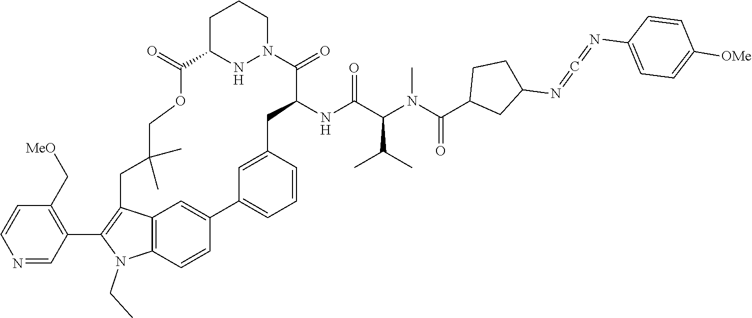 [SMILES: CCn1c(-c2cnccc2COC)c2c3cc(ccc31)-c1cccc(c1)C[C@H](NC(=O)[C@H](C(C)C)N(C)C(=O)C1CCC(N=C=Nc3ccc(OC)cc3)C1)C(=O)N1CCC[C@H](N1)C(=O)OCC(C)(C)C2] |
| 72 A and B | 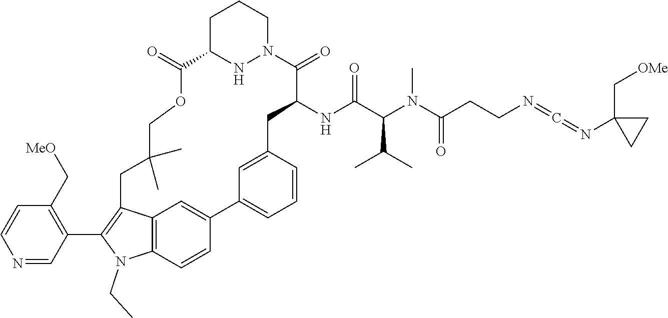 [SMILES: CCn1c(-c2cnccc2COC)c2c3cc(ccc31)-c1cccc(c1)C[C@H](NC(=O)[C@H](C(C)C)N(C)C(=O)CCN=C=NC1(COC)CC1)C(=O)N1CCC[C@H](N1)C(=O)OCC(C)(C)C2] |
| 73 | 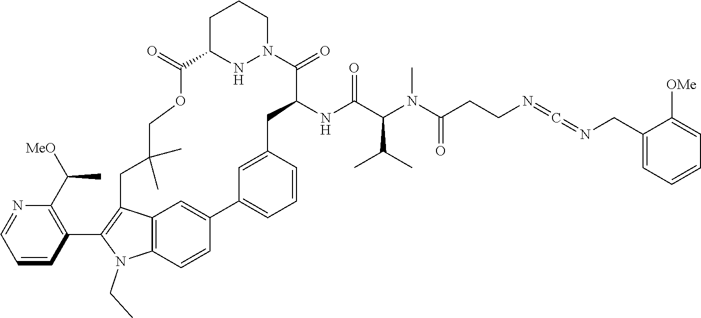 |
| 74 | 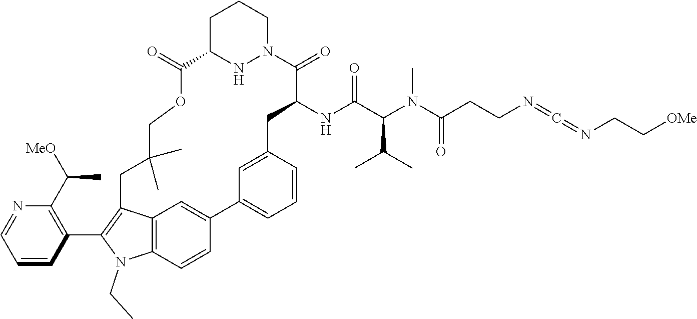 |
| 75 | 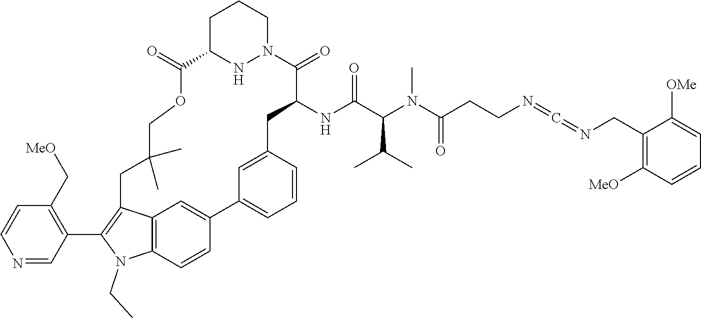 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| 76 | 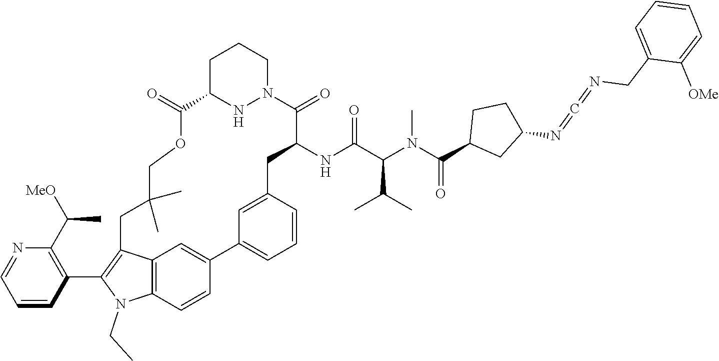 |
| 77 | 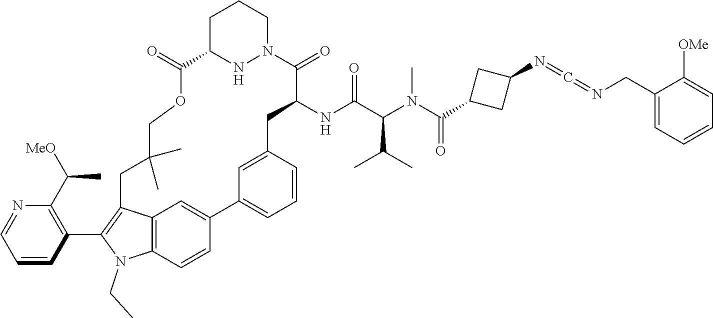 |
| 78 | 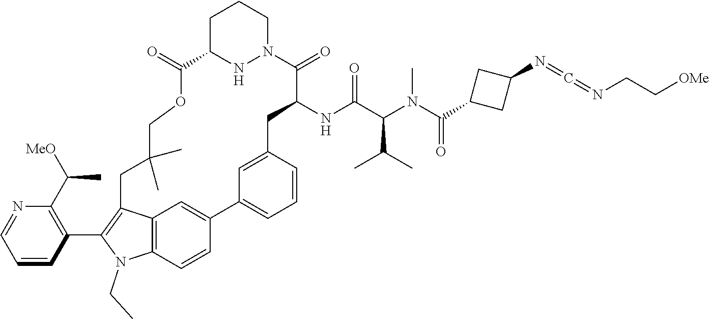 |
| 79 A and B | 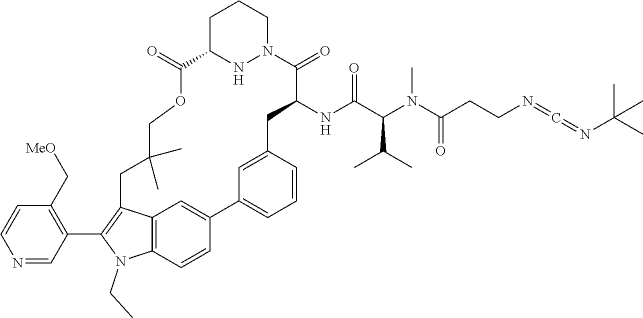 [SMILES: CCn1c(-c2cnccc2COC)c2c3cc(ccc31)-c1cccc(c1)C[C@H](NC(=O)[C@H](C(C)C)N(C)C(=O)CCN=C=NC(C)(C)C)C(=O)N1CCC[C@H](N1)C(=O)OCC(C)(C)C2] |
| 80 | 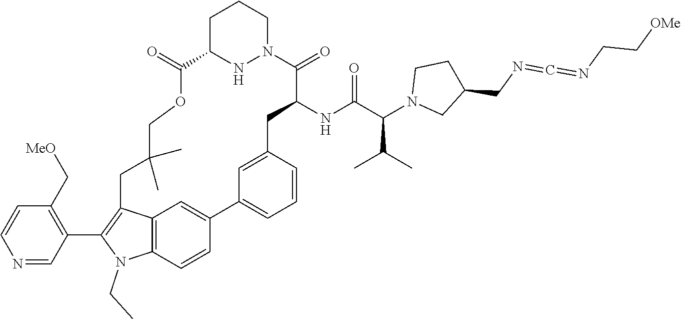 [SMILES: CCn1c(-c2cnccc2COC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| 81 | 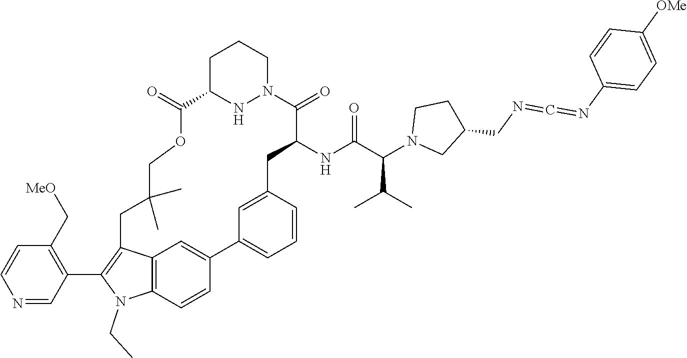 [SMILES: CCn1c(-c2cnccc2COC)c2c3cc(ccc31)-c1cccc(c1)C[C@H](NC(=O)[C@H](C(C)C)N1CC[C@H](CN=C=Nc3ccc(OC)cc3)C1)C(=O)N1CCC[C@H](N1)C(=O)OCC(C)(C)C2] |
| 82 A and B | 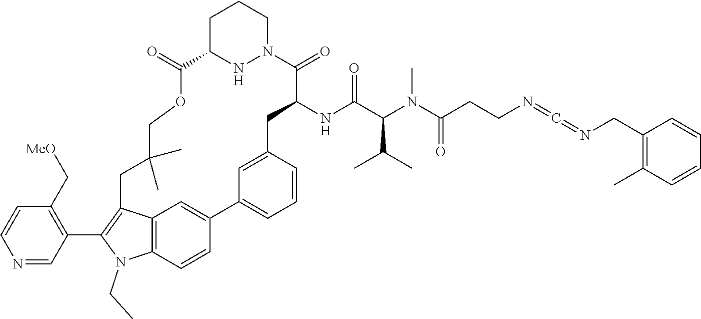 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1C)C(=O)N1CCC[C@H](N1)C(=O)OCC(C)(C)C2] |
| 83 | 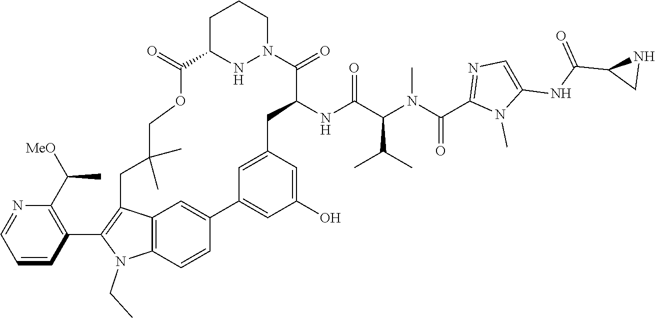 |
| 84 | 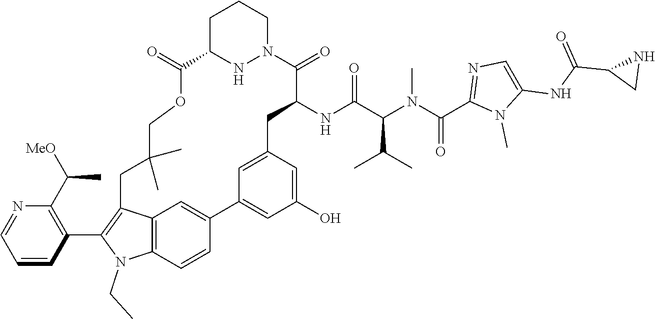 |
| 85 | 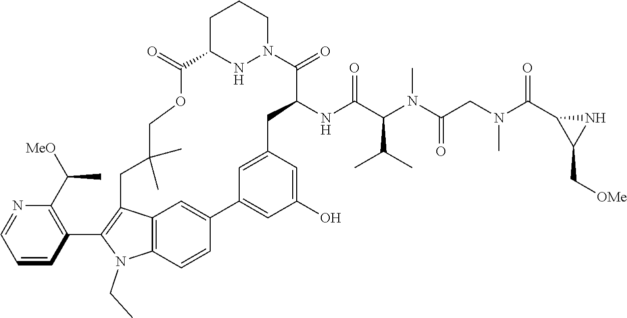 |
| 86 | 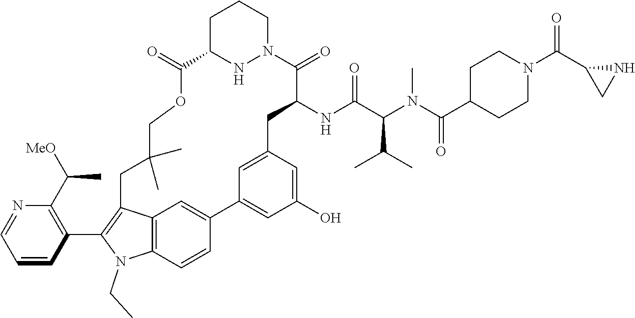 |
| 87 | 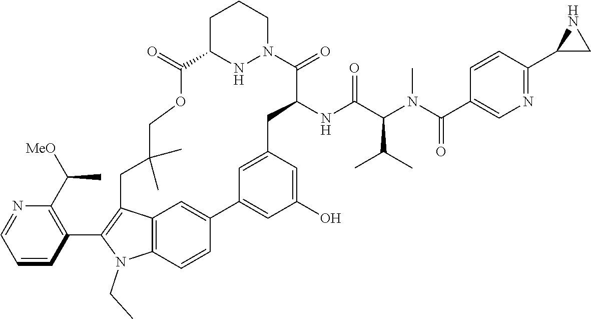 |
| 88 | 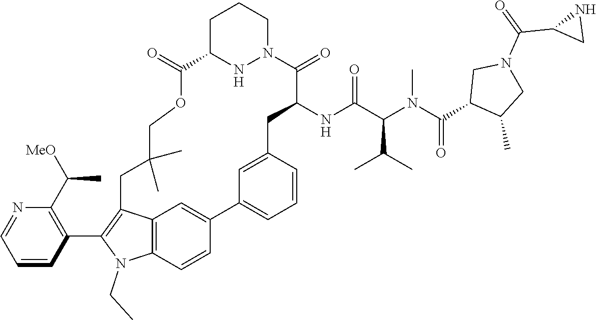 |
| 89 | 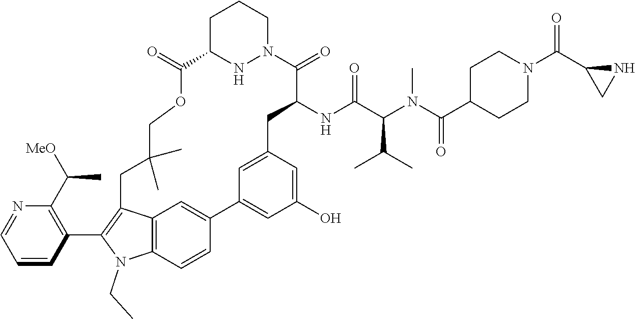 |
| 90 | 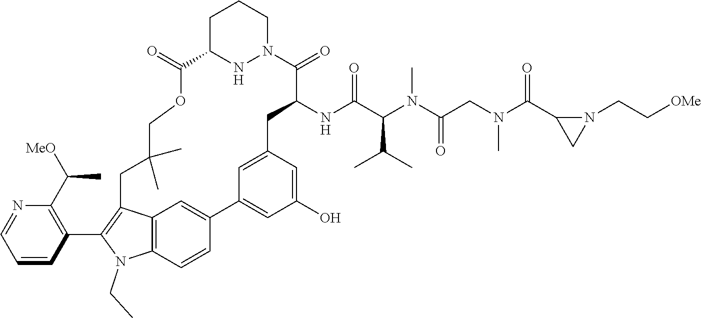 |
| 91* | 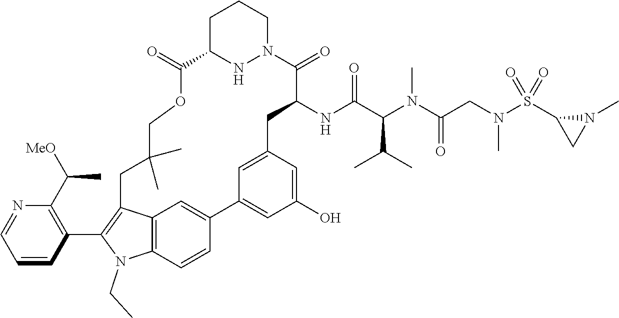 |
| 92* | 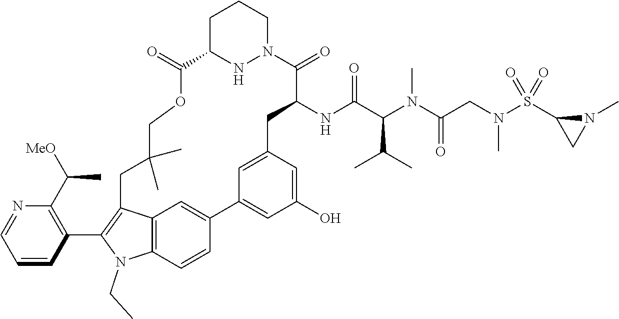 |
| 93 | 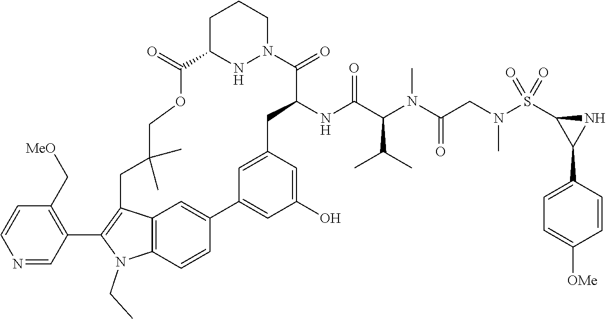 [SMILES: CCn1c(-c2cnccc2COC)c2c3cc(ccc31)-c1cc(O)cc(c1)C[C@H](NC(=O)[C@H](C(C)C)N(C)C(=O)CN(C)S(=O)(=O)[C@H]1N[C@H]1c1ccc(OC)cc1)C(=O)N1CCC[C@H](N1)C(=O)OCC(C)(C)C2] |
| 94 | 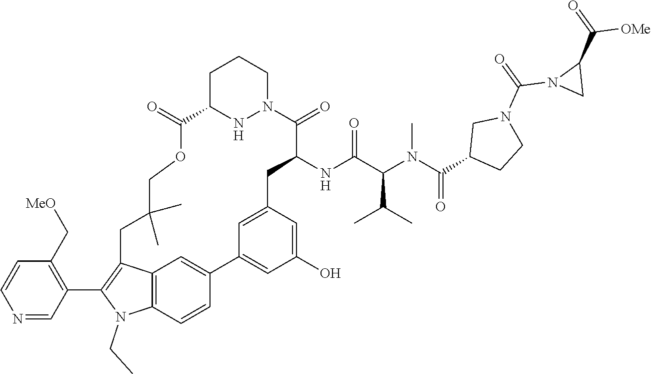 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@@H]3C(=O)OC)C1)C(=O)N1CCC[C@H](N1)C(=O)OCC(C)(C)C2] |
| 95 | 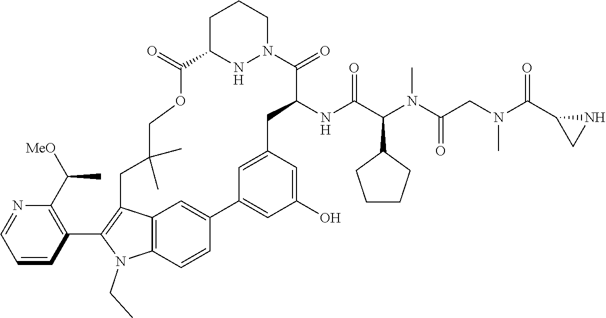 |
| 96 | 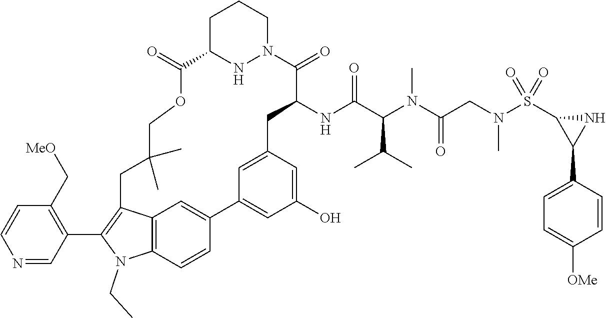 [SMILES: CCn1c(-c2cnccc2COC)c2c3cc(ccc31)-c1cc(O)cc(c1)C[C@H](NC(=O)[C@H](C(C)C)N(C)C(=O)CN(C)S(=O)(=O)[C@@H]1N[C@H]1c1ccc(OC)cc1)C(=O)N1CCC[C@H](N1)C(=O)OCC(C)(C)C2] |
| 97 | 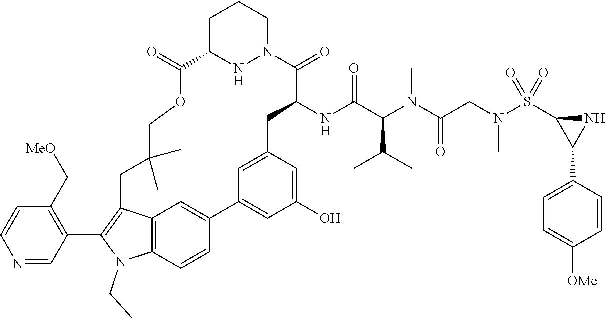 [SMILES: CCn1c(-c2cnccc2COC)c2c3cc(ccc31)-c1cc(O)cc(c1)C[C@H](NC(=O)[C@H](C(C)C)N(C)C(=O)CN(C)S(=O)(=O)[C@H]1N[C@@H]1c1ccc(OC)cc1)C(=O)N1CCC[C@H](N1)C(=O)OCC(C)(C)C2] |
| 98 | 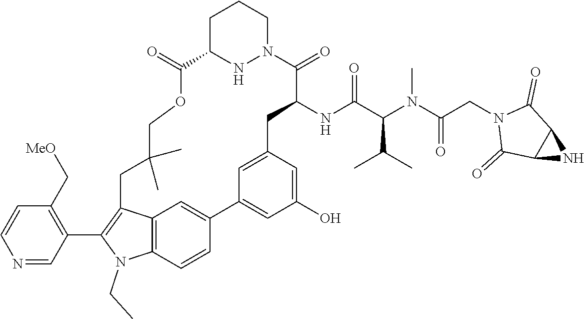 [SMILES: CCn1c(-c2cnccc2COC)c2c3cc(ccc31)-c1cc(O)cc(c1)C[C@H](NC(=O)[C@H](C(C)C)N(C)C(=O)CN1C(=O)[C@H]3N[C@H]3C1=O)C(=O)N1CCC[C@H](N1)C(=O)OCC(C)(C)C2] |
| 99 | 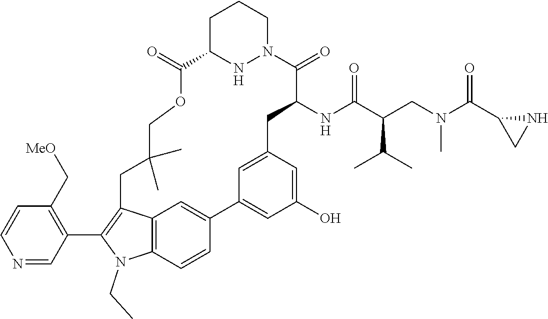 [SMILES: CCn1c(-c2cnccc2COC)c2c3cc(ccc31)-c1cc(O)cc(c1)C[C@H](NC(=O)[C@@H](CN(C)C(=O)[C@H]1CN1)C(C)C)C(=O)N1CCC[C@H](N1)C(=O)OCC(C)(C)C2] |
| 100 | 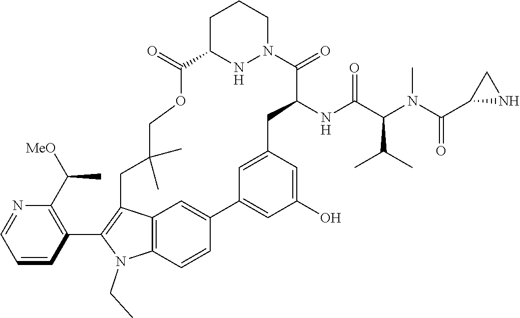 |
| 101 | 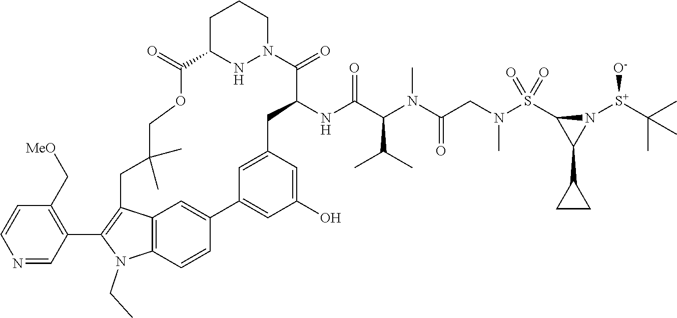 |
| 102 | 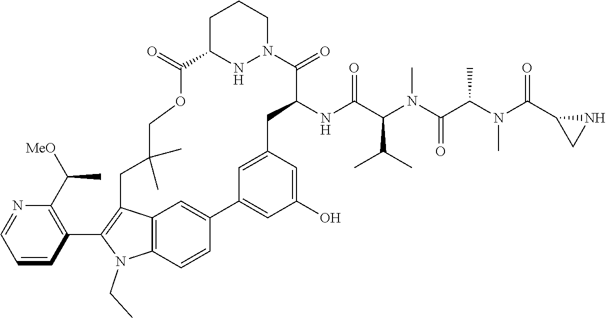 |
| 103 | 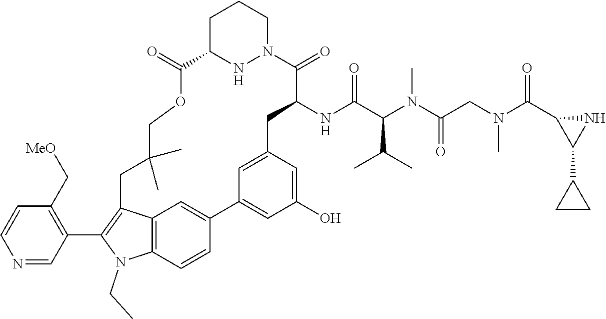 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C1CC1)C(=O)N1CCC[C@H](N1)C(=O)OCC(C)(C)C2] |
| 104 | 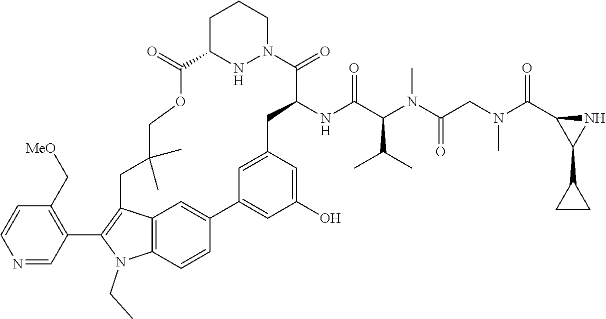 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C1CC1)C(=O)N1CCC[C@H](N1)C(=O)OCC(C)(C)C2] |
| 105 | 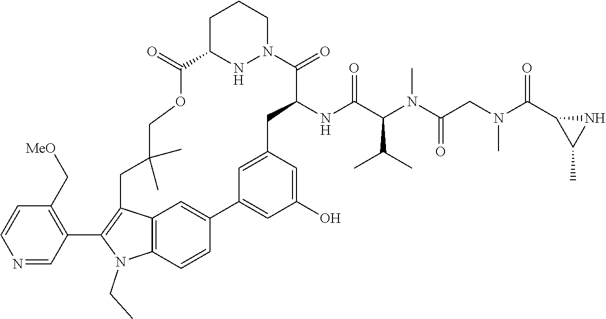 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C)C(=O)N1CCC[C@H](N1)C(=O)OCC(C)(C)C2] |
| 106 | 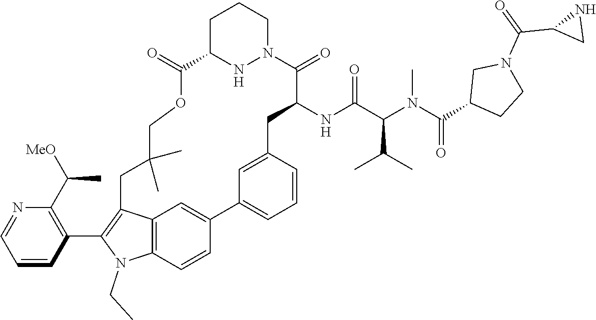 |
| 107 | 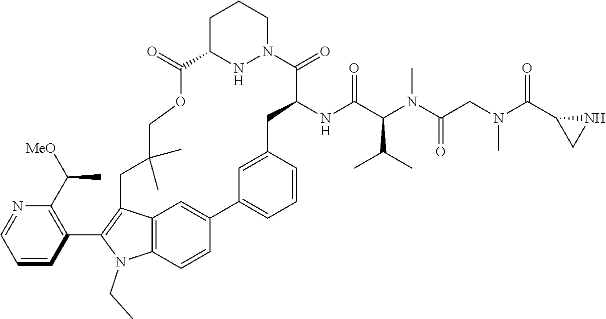 |
| 108* | 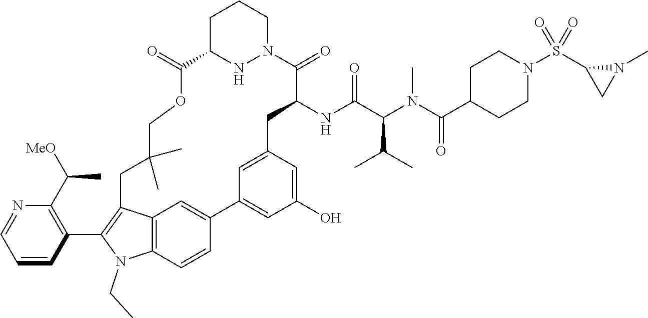 |
| 109 | 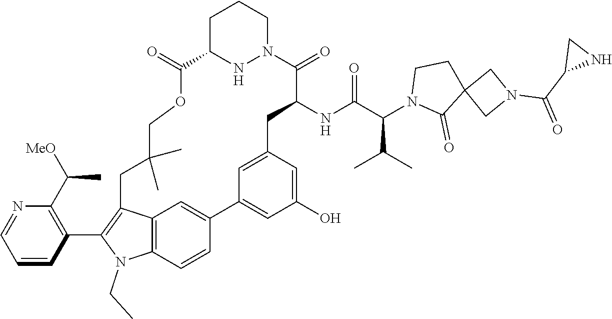 |
| 110 | 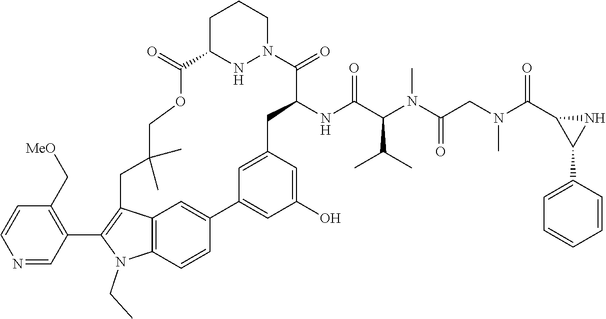 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccccc1)C(=O)N1CCC[C@H](N1)C(=O)OCC(C)(C)C2] |
| 111 | 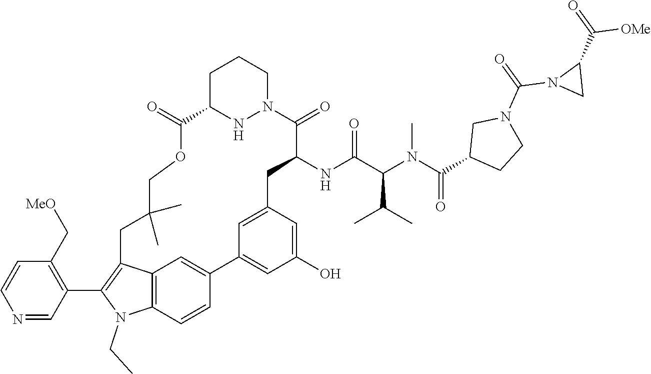 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@H]3C(=O)OC)C1)C(=O)N1CCC[C@H](N1)C(=O)OCC(C)(C)C2] |
| 112 | 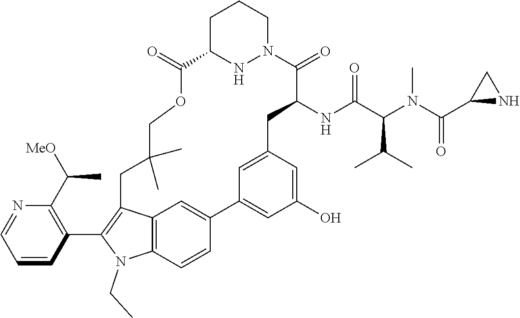 |
| 113 | 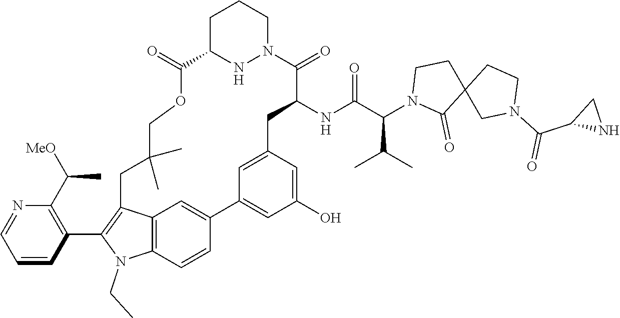 |
| 114 | 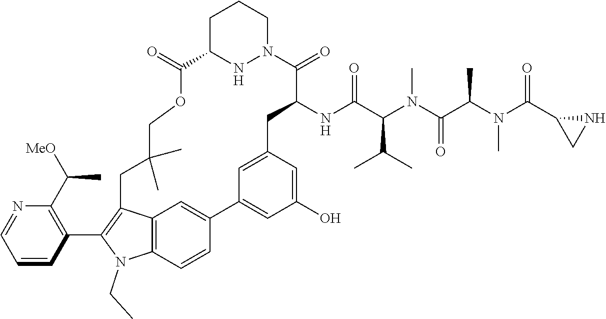 |
| 115 | 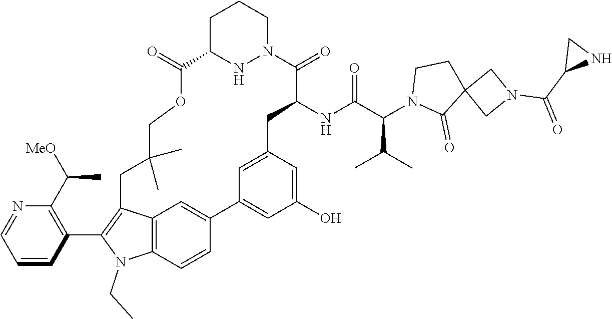 |
| 116 | 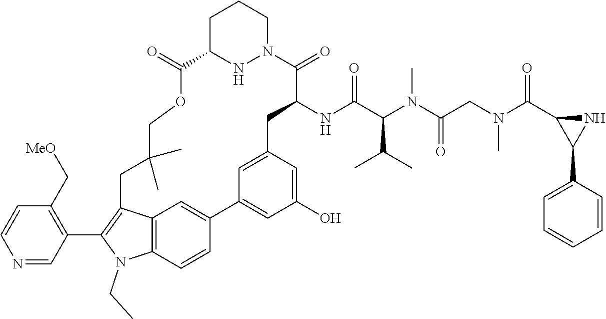 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccccc1)C(=O)N1CCC[C@H](N1)C(=O)OCC(C)(C)C2] |
| 117* | 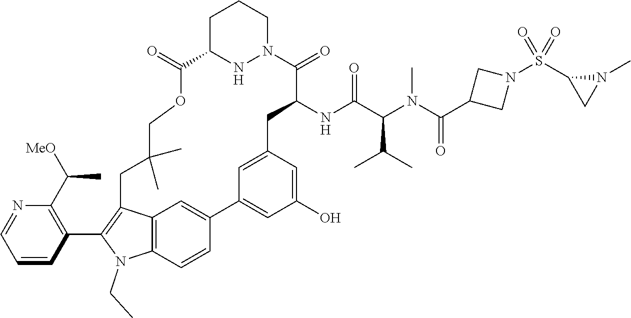 |
| 118* | 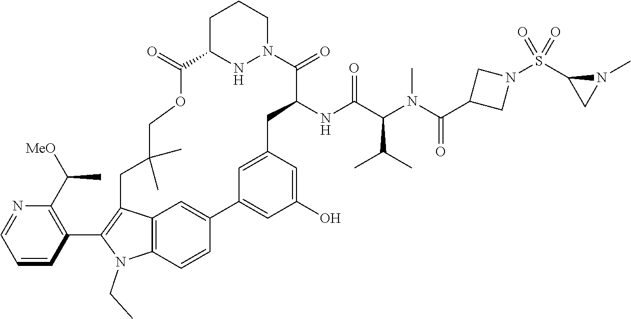 |
| 119* | 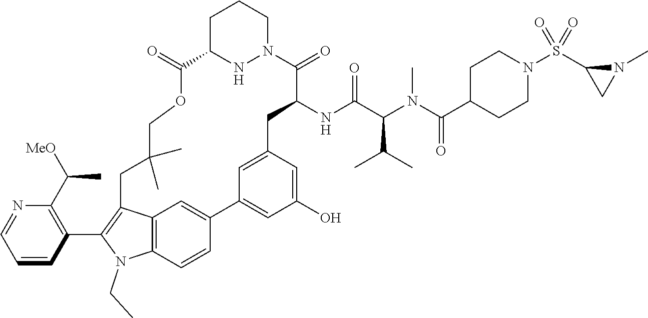 |
| 120 | 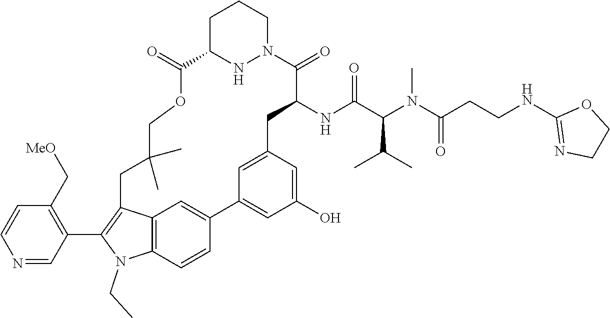 [SMILES: CCn1c(-c2cnccc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| 121 | 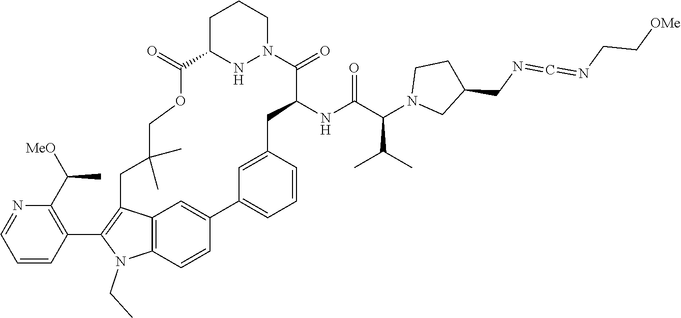 [SMILES: CCn1c(-c2cccnc2[C@H](C)OC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| 122 | 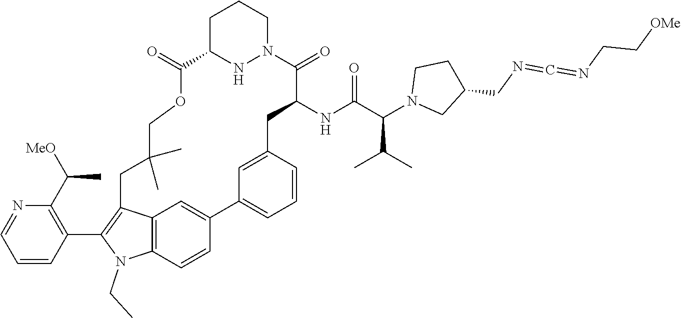 [SMILES: CCn1c(-c2cccnc2[C@H](C)OC)c2c3cc(ccc31)-c1cccc(c1)C[C@H](NC(=O)[C@H](C(C)C)N1CC[C@H](CN=C=NCCOC)C1)C(=O)N1CCC[C@H](N1)C(=O)OCC(C)(C)C2] |
| 123 | 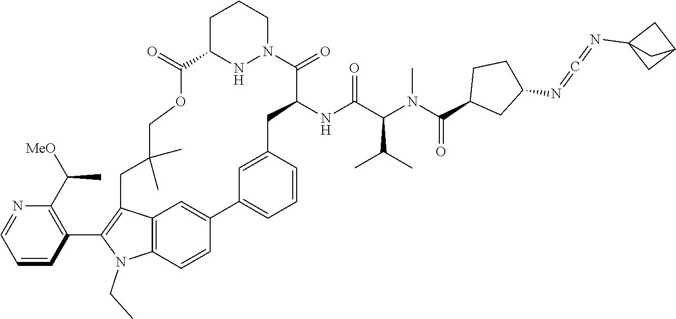 |
| 124 | 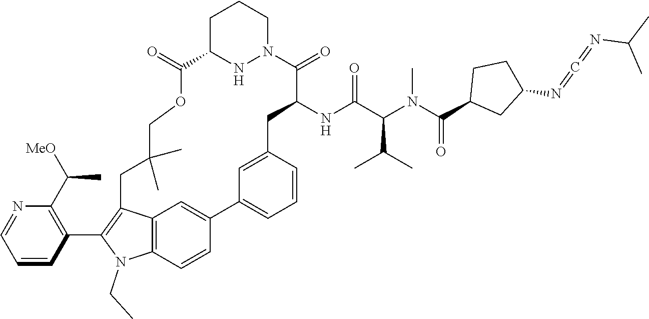 |
| 125 | 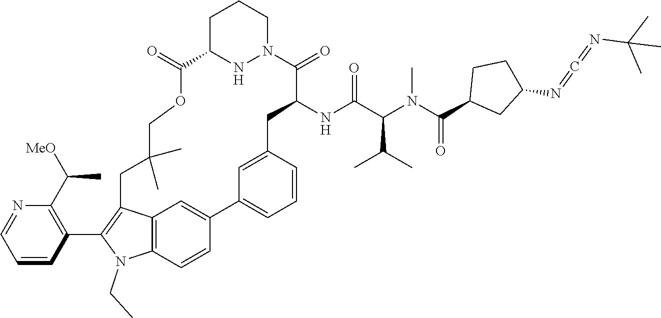 |
| 126 | 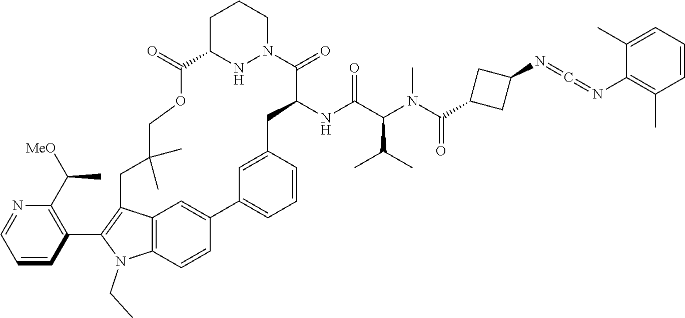 |
| 127 | 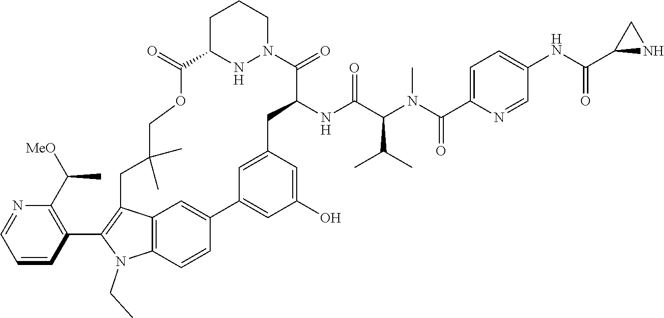 |
| 128 | 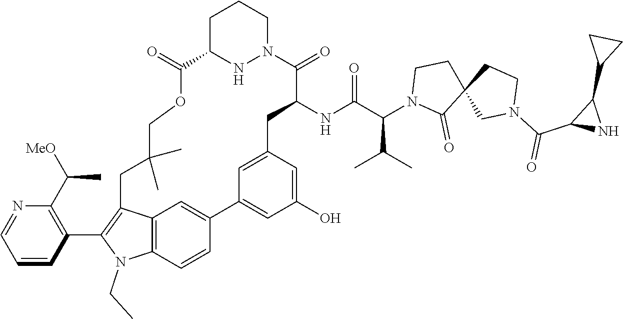 |
| 129 | 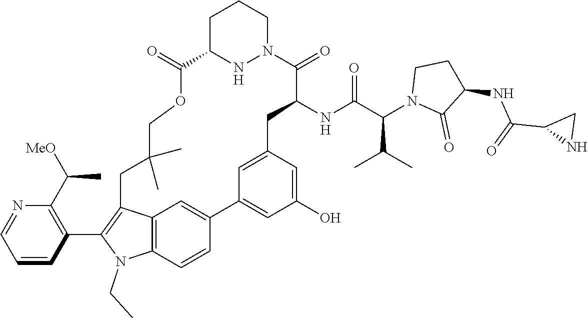 |
| 130 | 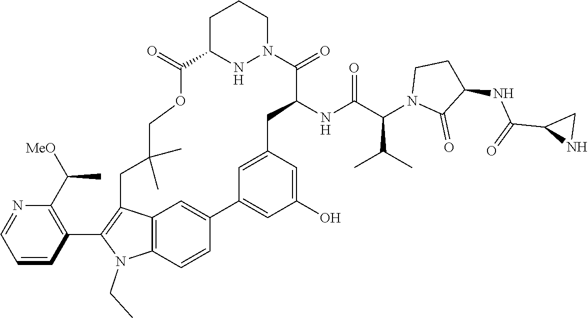 |
| 131 | 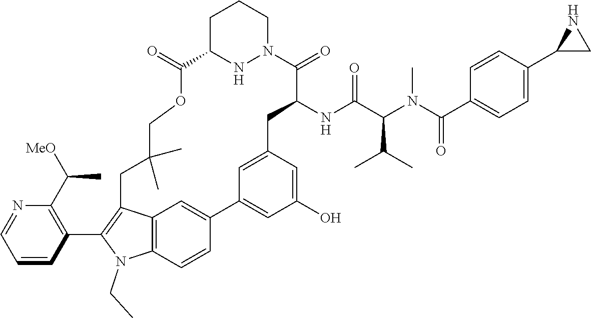 |
| 132 | 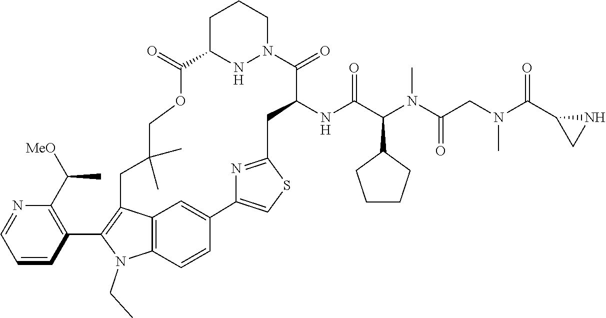 |
| 133 | 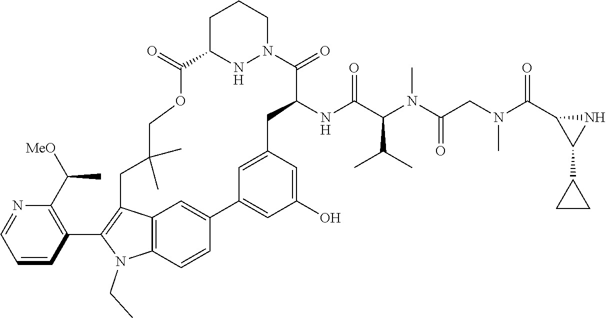 |
| 134 | 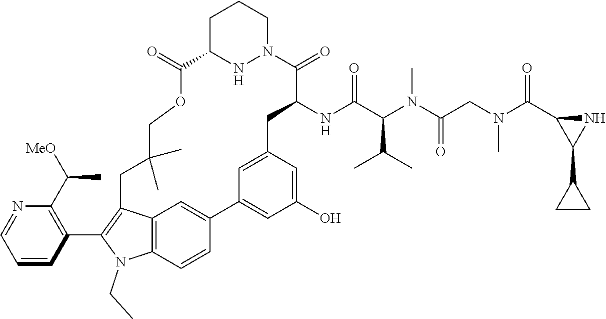 |
| 135 | 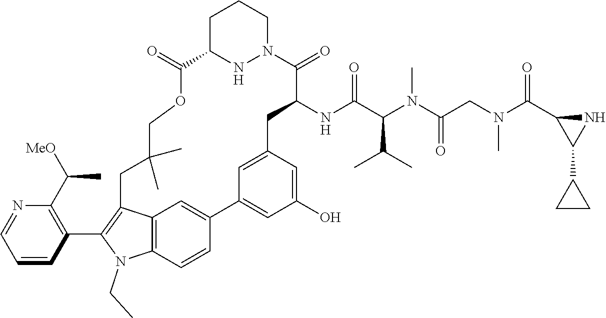 |
| 136 | 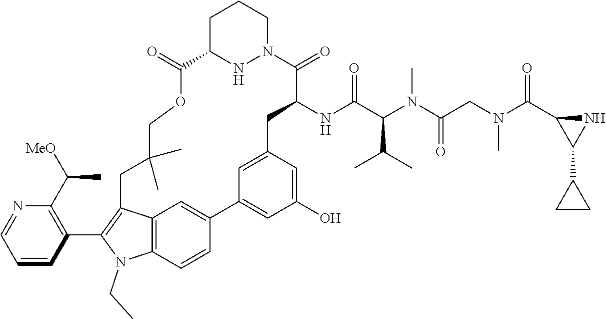 |
| 137 | 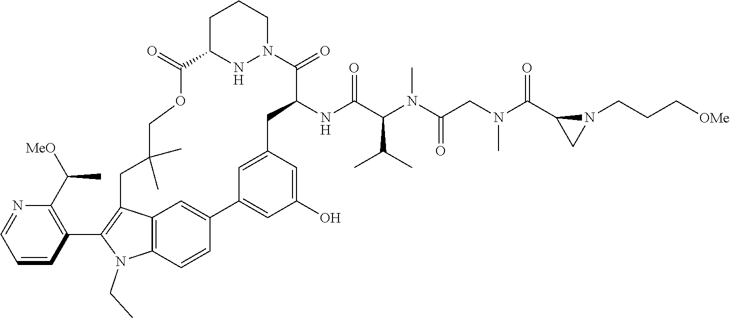 |
| 138 | 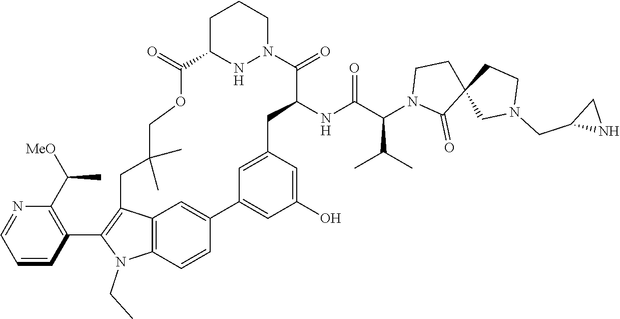 |
| 139 | 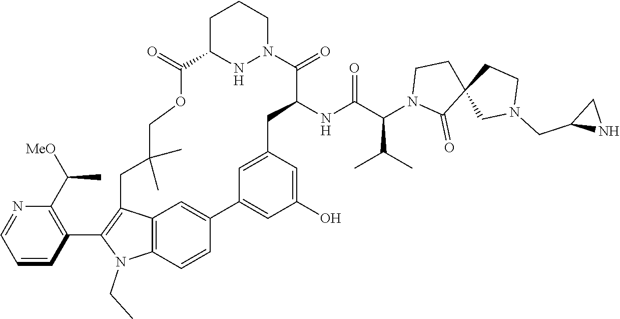 |
| 140 | 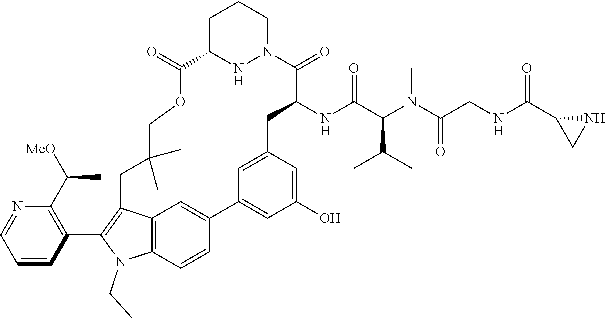 |
| 141 | 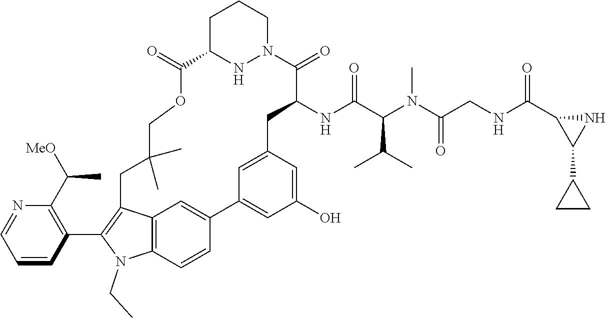 |
| 142 | 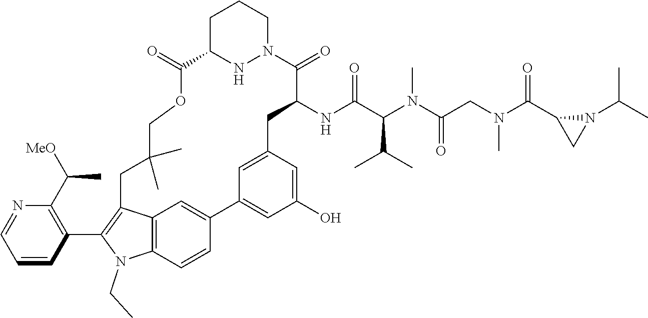 |
| 143 | 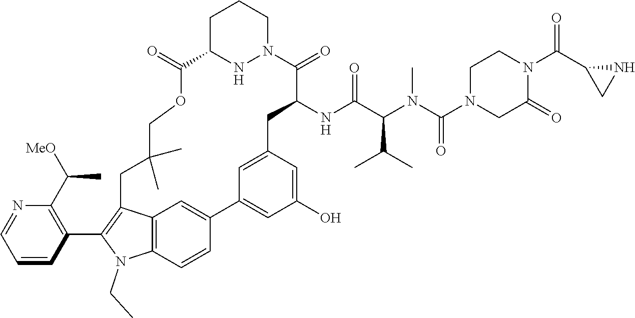 |
| 144 | 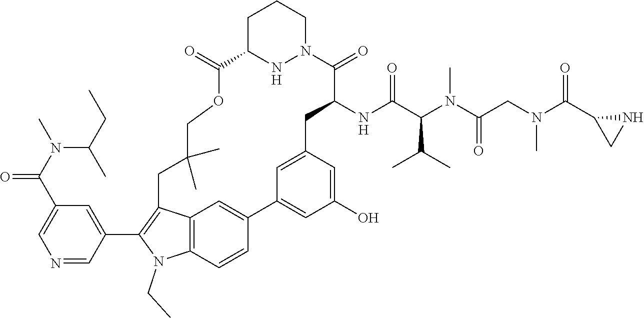 [SMILES: CCC(C)N(C)C(=O)c1cncc(-c2c3c4cc(ccc4n2CC)-c2cc(O)cc(c2)C[C@H](NC(=O)[C@H](C(C)C)N(C)C(=O)CN(C)C(=O)[C@H]2CN2)C(=O)N2CCC[C@H](N2)C(=O)OCC(C)(C)C3)c1] |
| 145 | 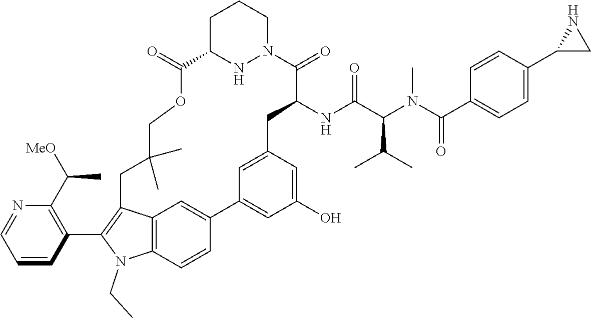 |
| 146 | 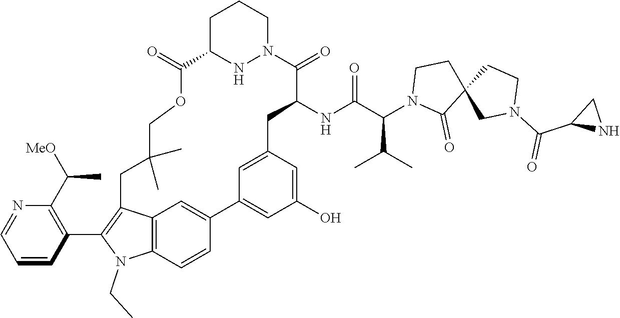 |
| 147 | 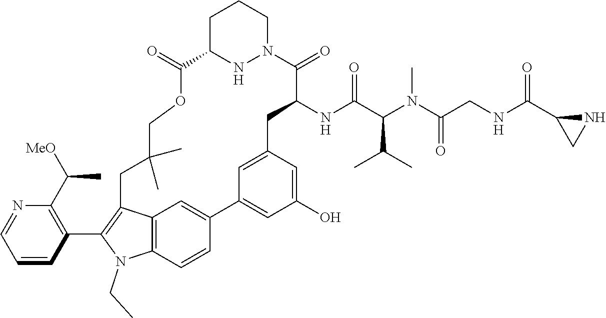 |
| 148 | 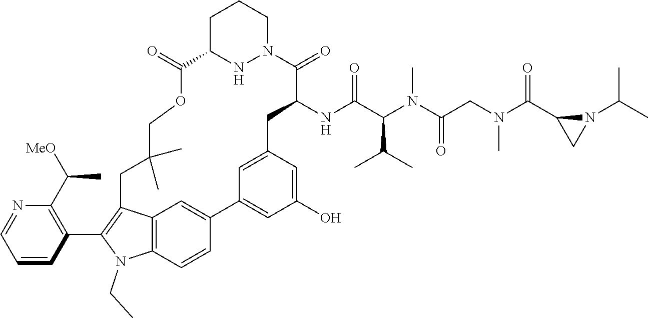 |
| 149 | 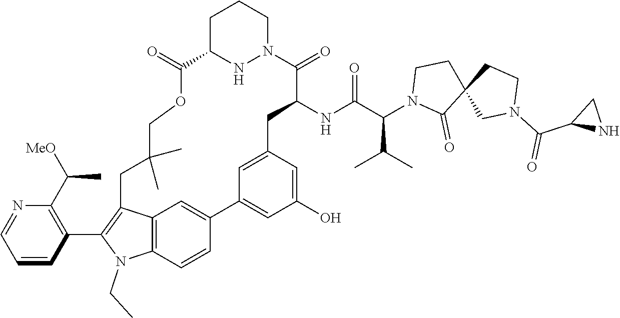 |
| 150 | 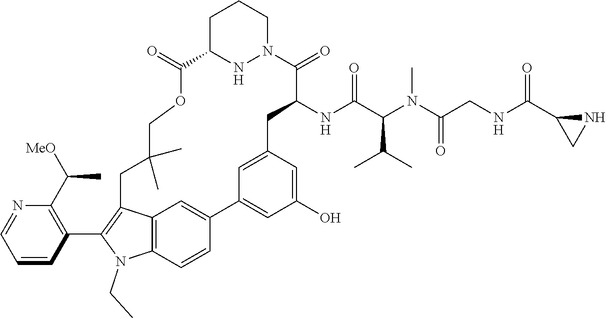 |
| 151 | 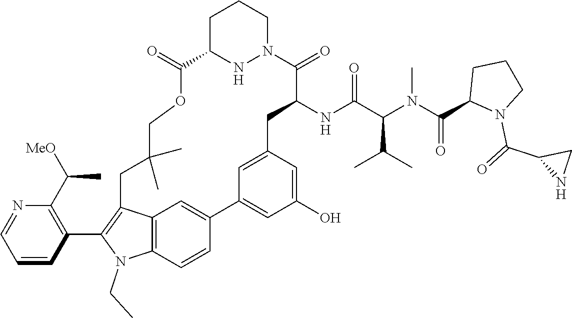 |
| 152 | 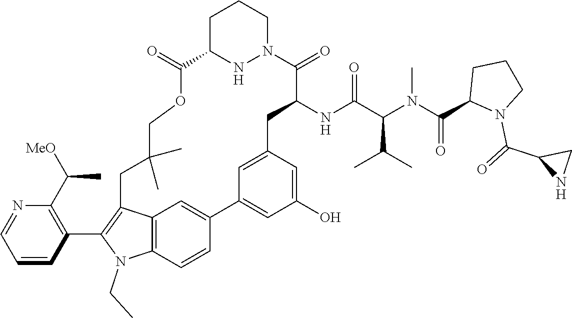 |
| 153 | 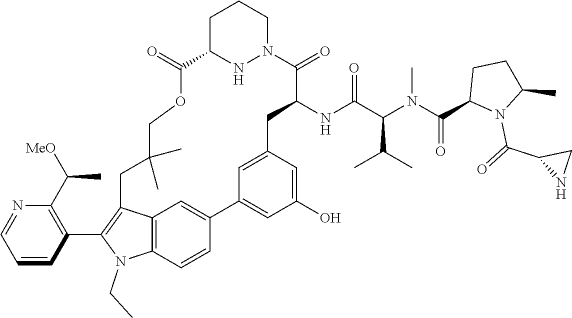 |
| 154 | 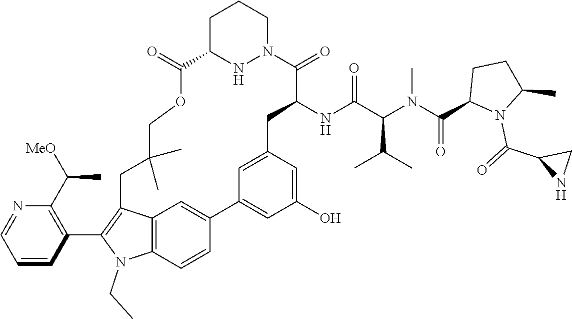 |
| 155 | 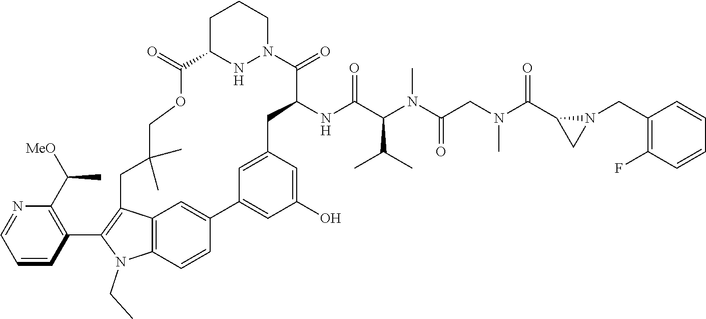 |
| 156 | 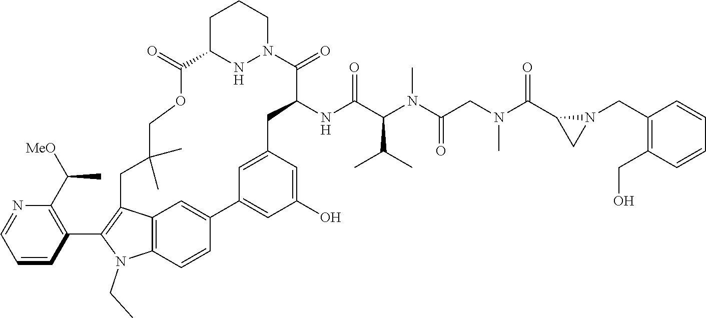 |
| 157 | 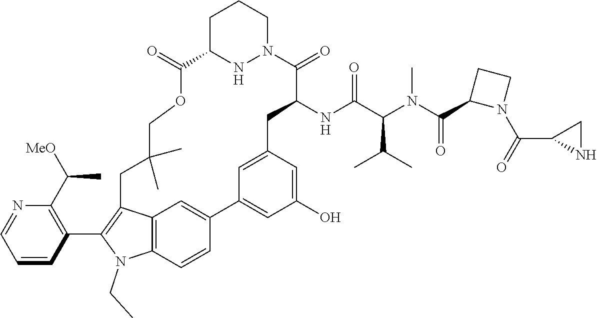 |
| 158 | 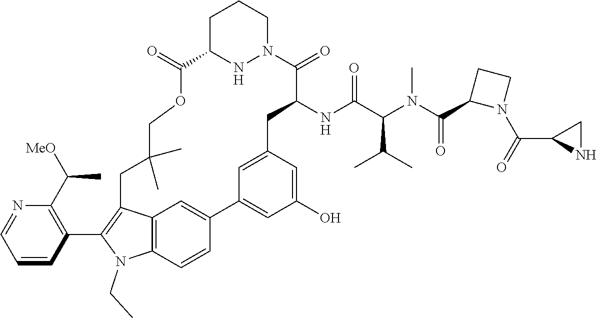 |
| 159 | 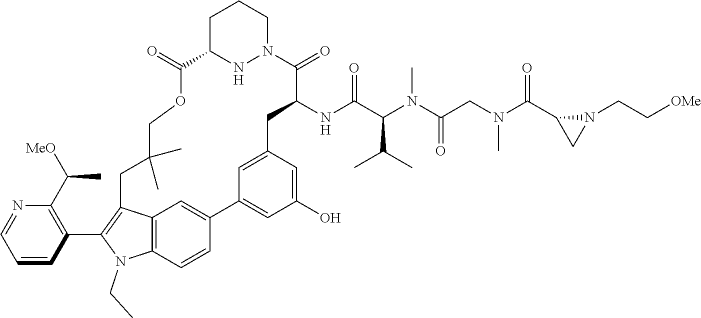 |
| 160 | 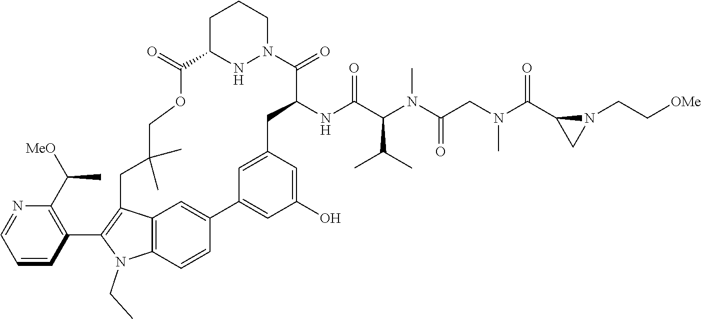 |
| 161 | 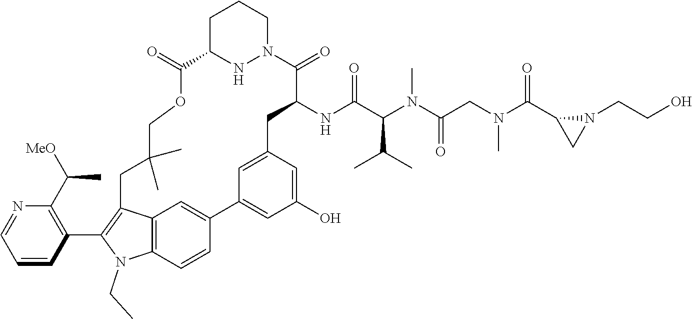 |
| 162 | 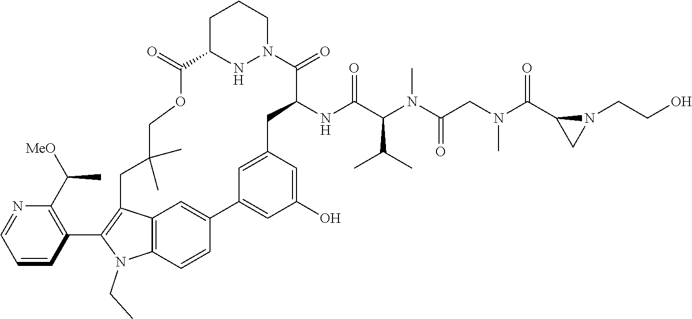 |
| 163 | 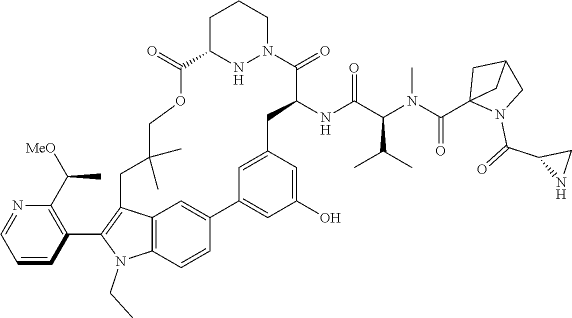 |
| 164 | 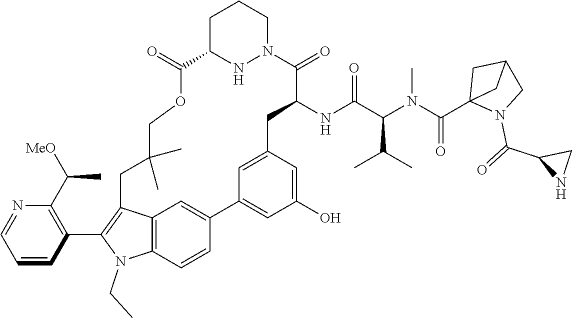 |
| 165 | 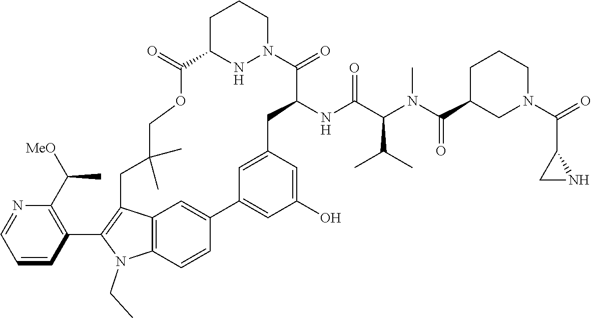 |
| 166 | 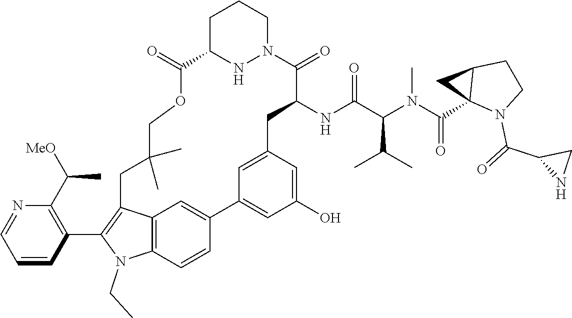 |
| 167 | 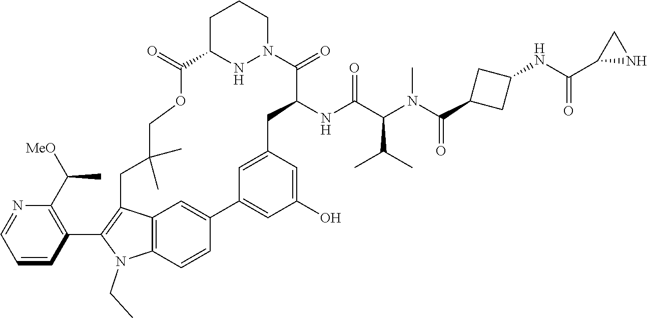 |
| 168 | 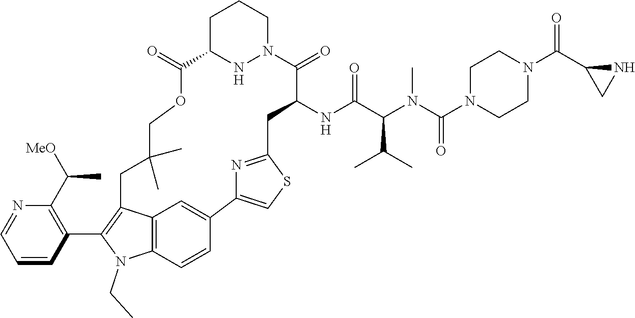 |
| 169 | 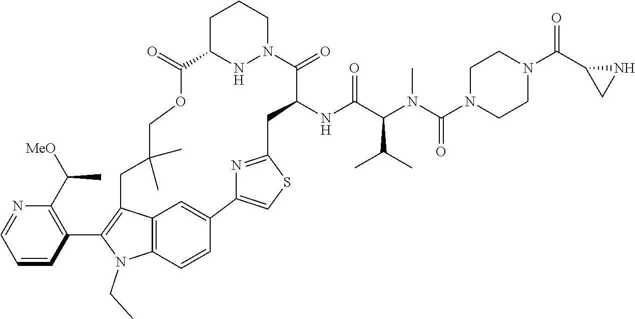 |
| 170 | 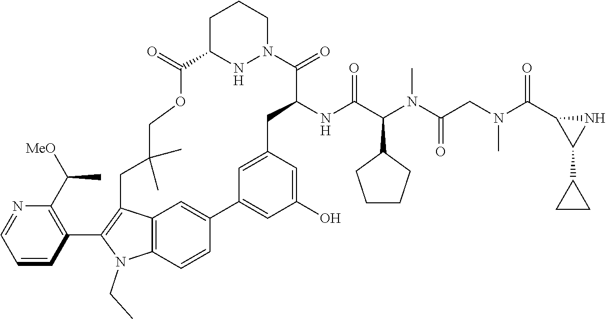 |
| 171 | 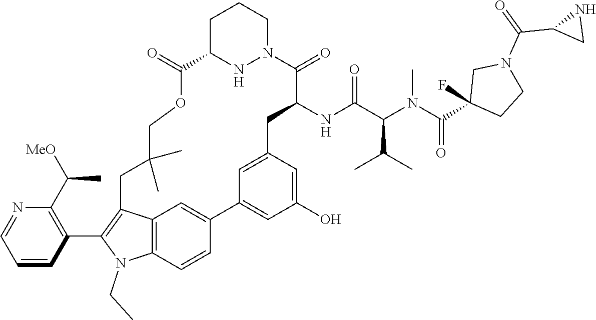 |
| 172 | 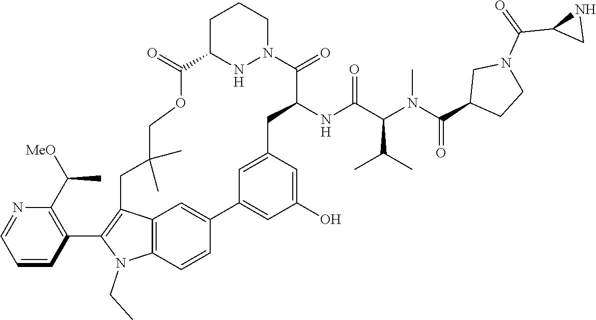 |
| 173 | 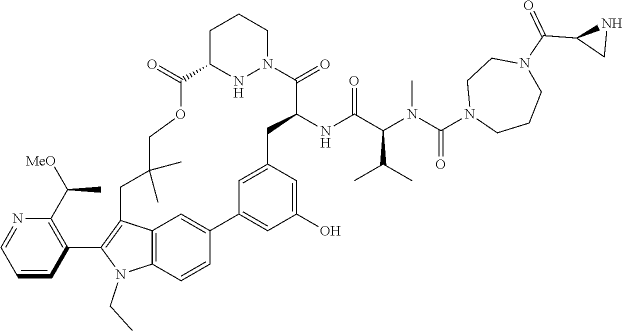 |
| 174 | 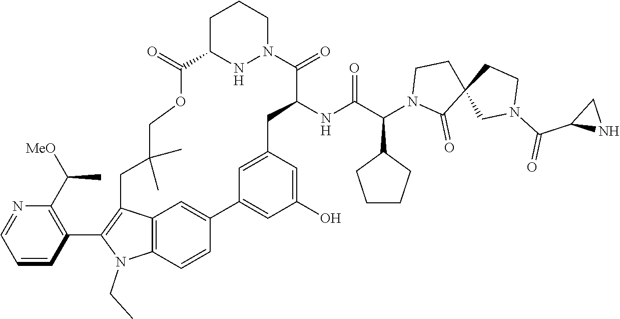 |
| 175 | 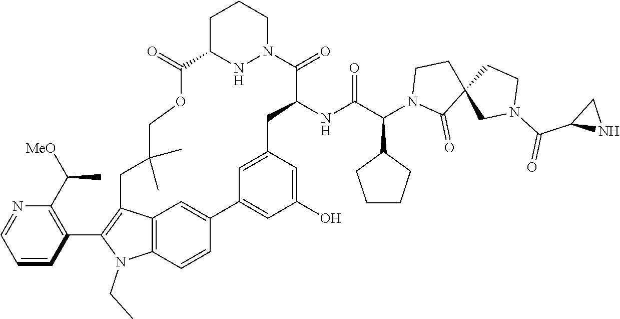 |
| 176 | 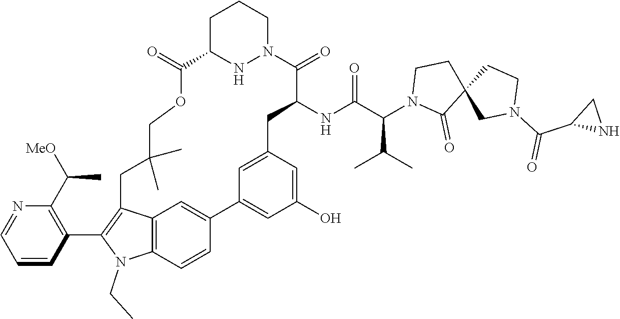 |
| 177 | 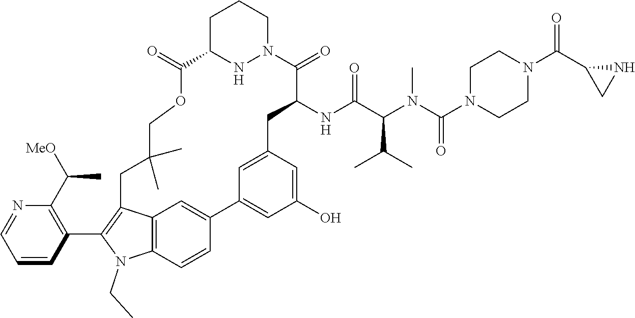 |
| 178 | 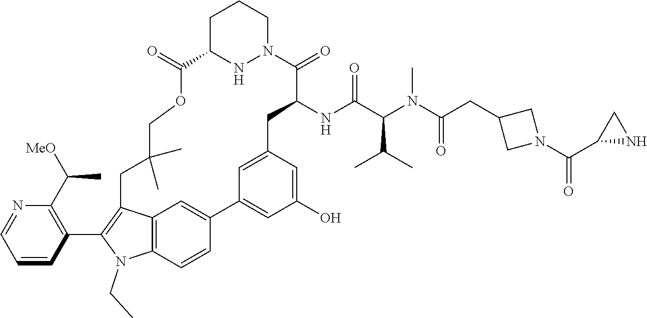 |
| 179 | 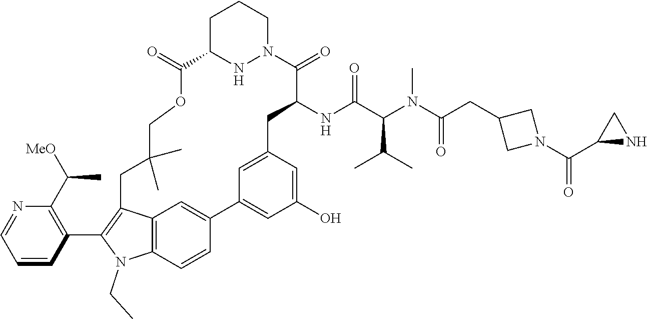 |
| 180 | 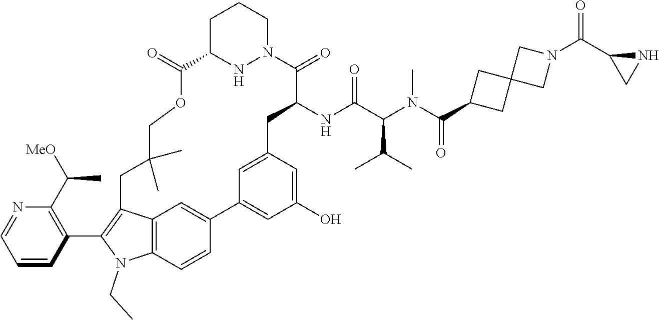 |
| 181 | 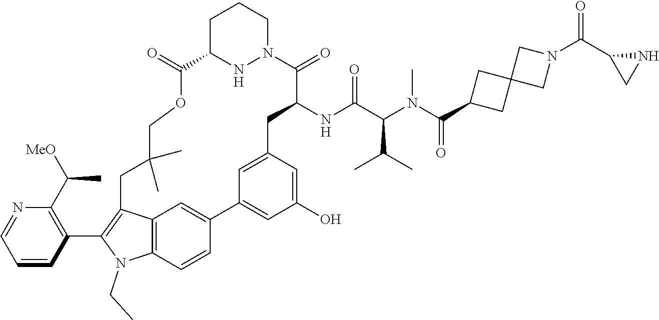 |
| 182 | 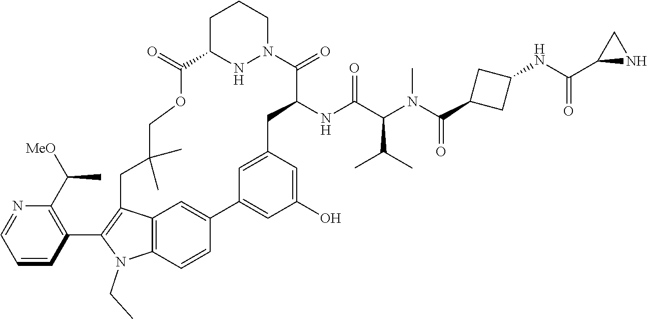 |
| 183 | 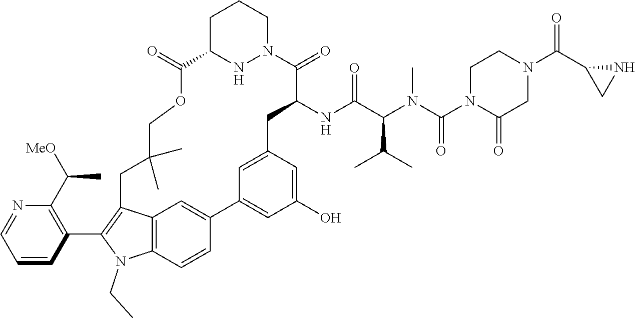 |
| 184 | 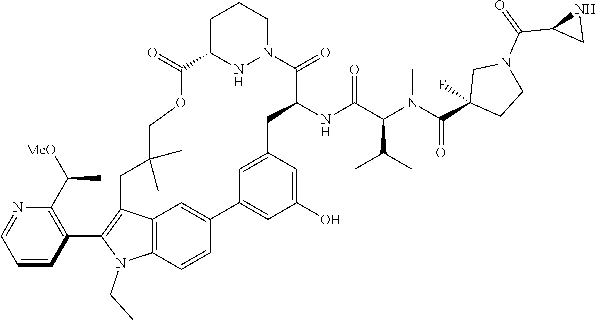 |
| 185 | 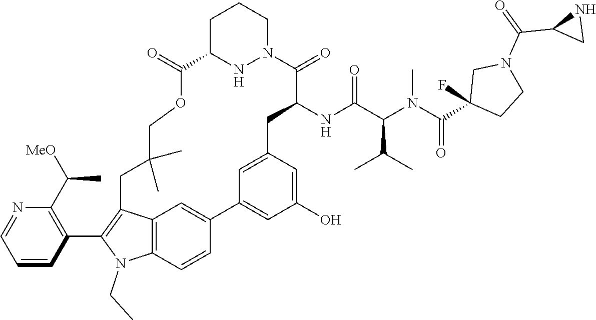 |
| 186 | 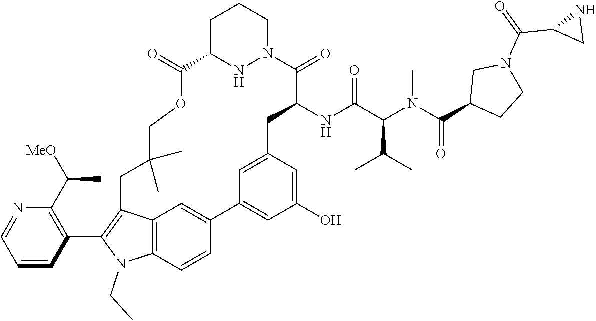 |
| 187 | 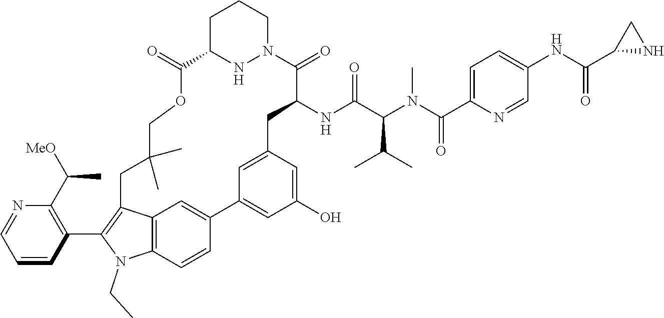 |
| 188 | 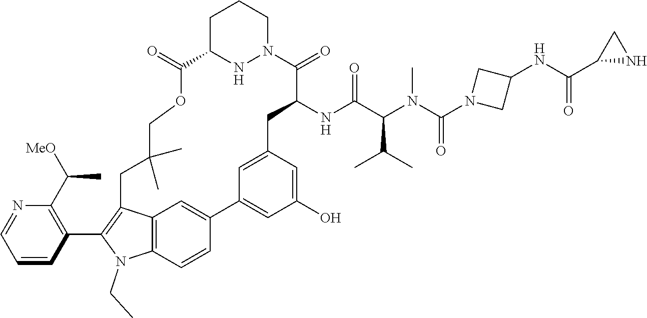 |
| 189 | 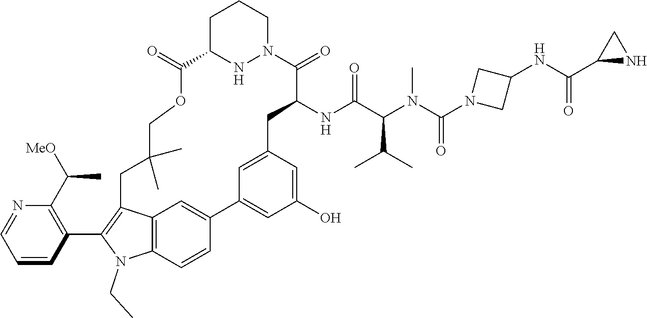 |
| 190 | 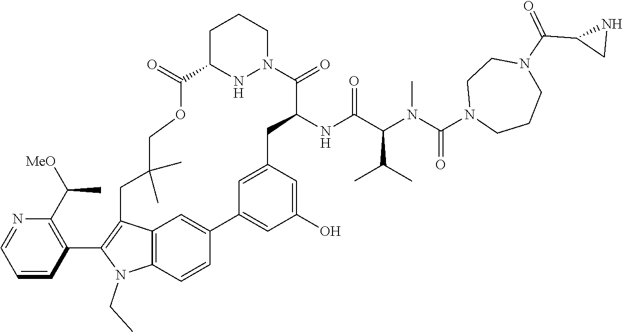 |
| 191 | 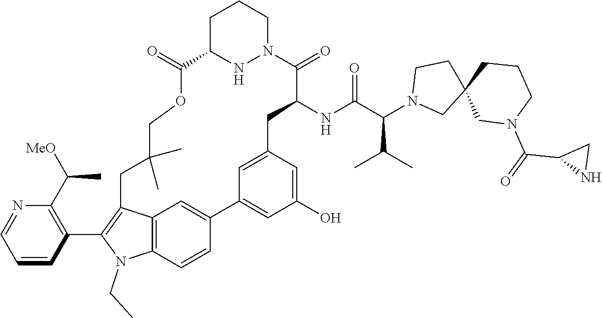 |
| 192 | 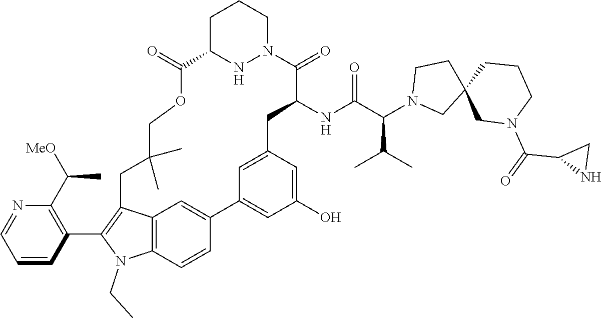 |
| 193 | 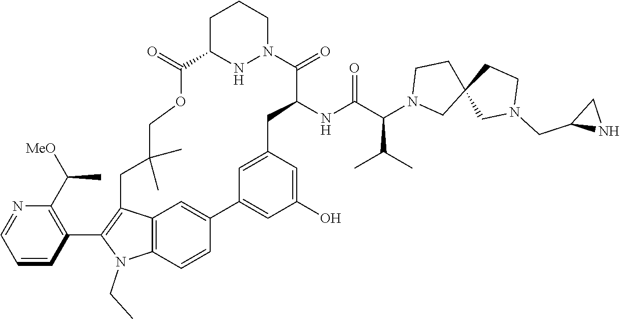 |
| 194 | 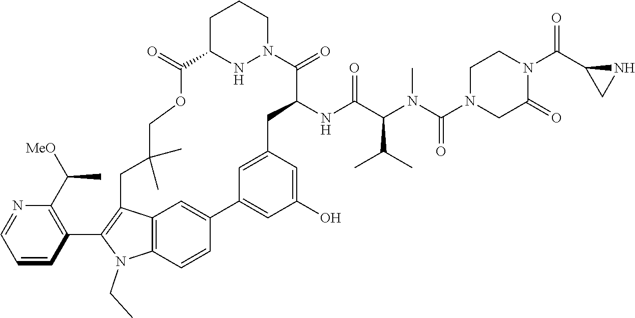 |
| 195 | 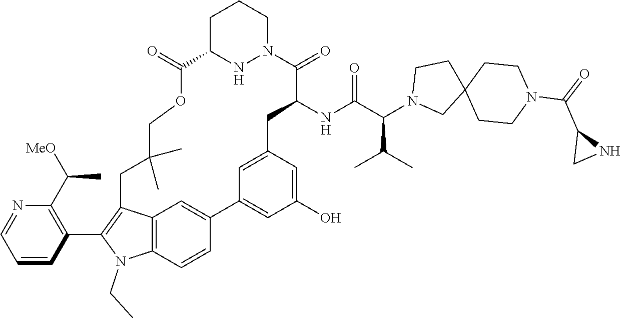 |
| 196 | 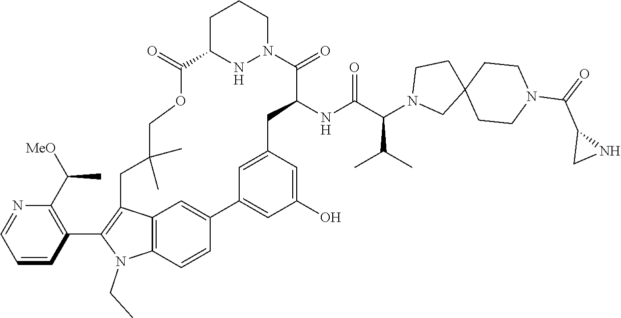 |
| 197 | 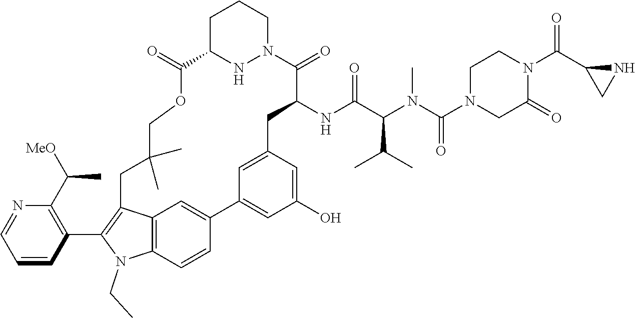 |
| 198 | 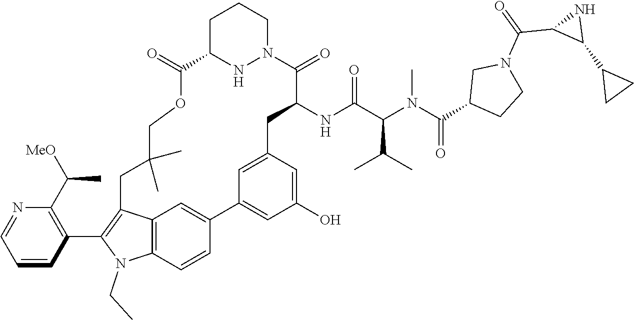 |
| 199 | 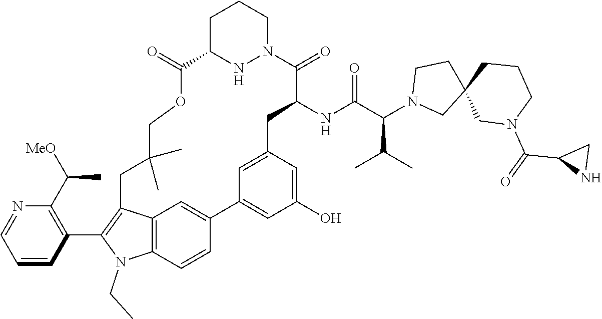 |
| 200 | 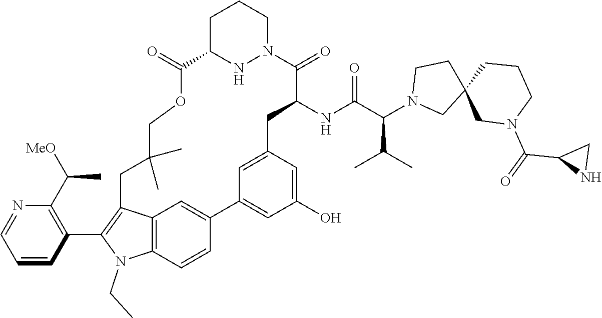 |
| 201 | 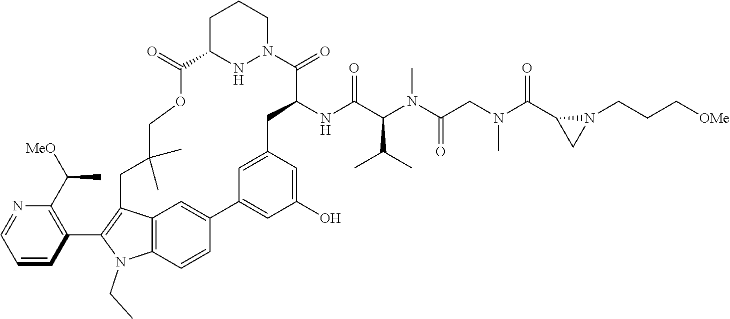 |
| 202 | 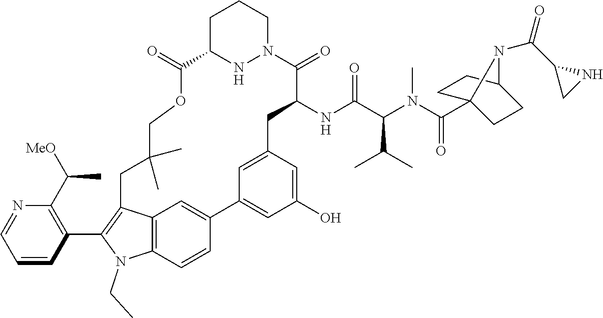 |
| 203 | 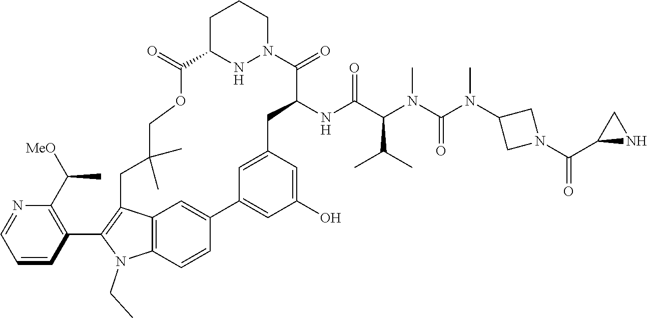 |
| 204 | 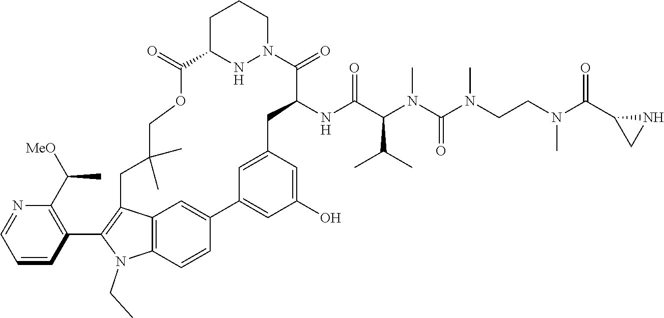 |
| 205 | 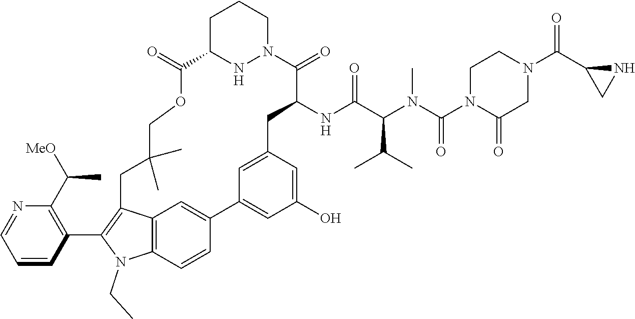 |
| 206 | 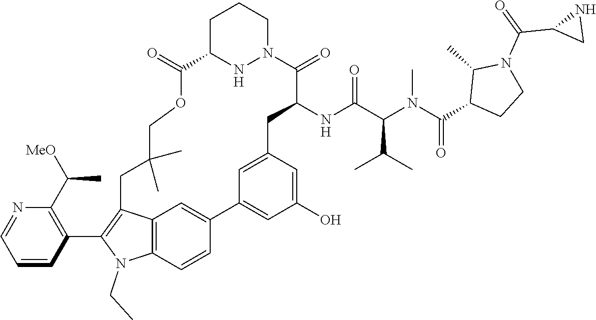 |
| 207 | 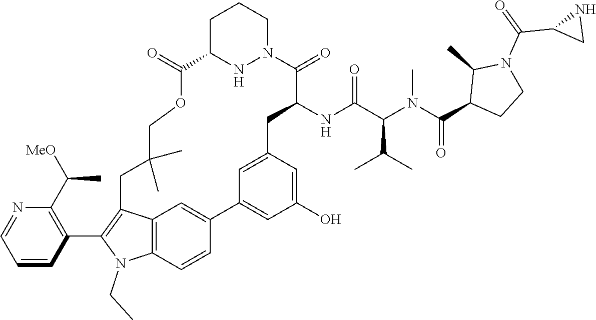 |
| 208 | 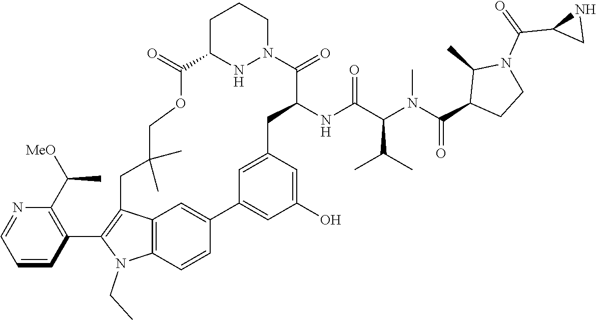 |
| 209 | 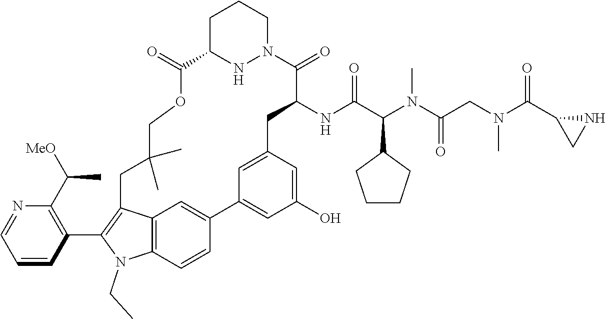 |
| 210 | 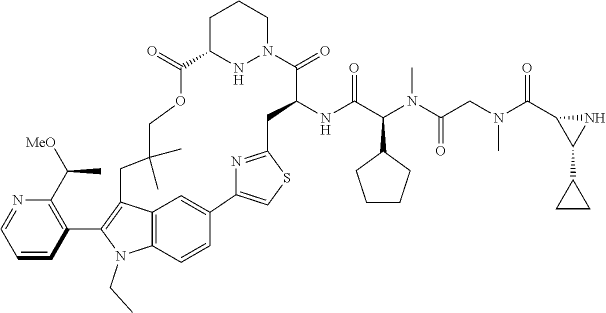 |
| 211 | 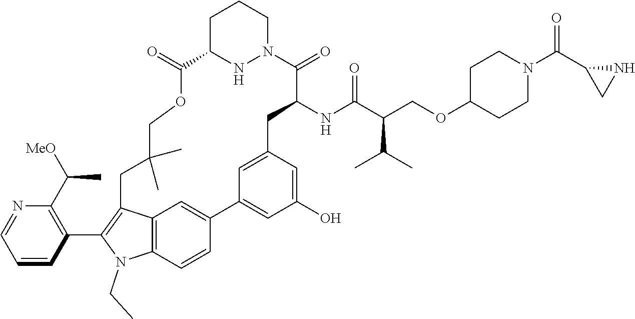 |
| 212 | 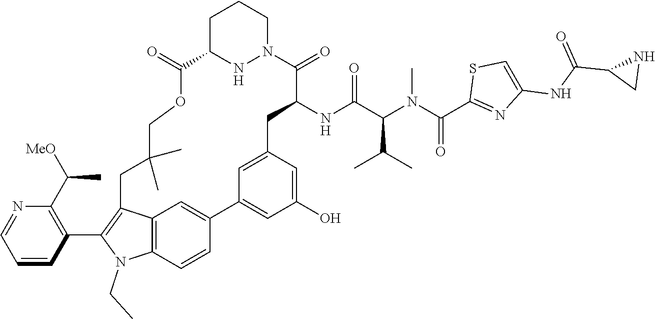 |
| 213 | 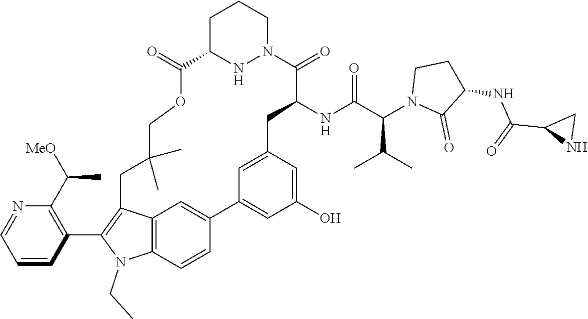 |
| 214 | 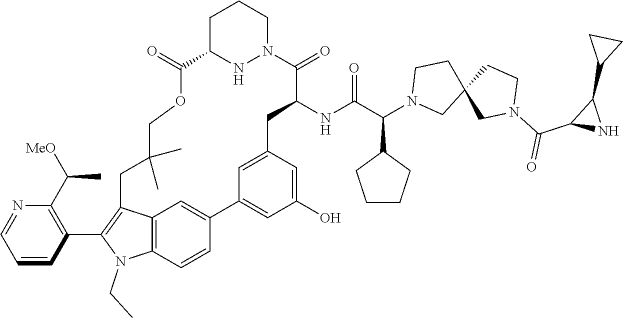 |
| 215 | 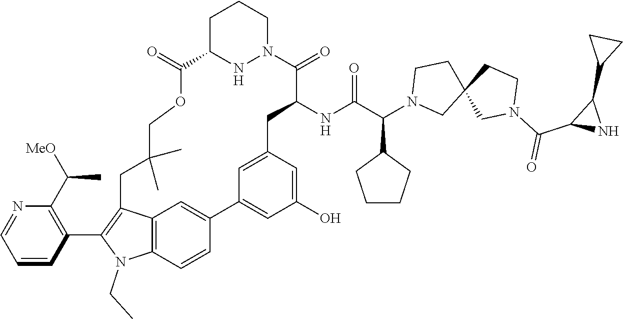 |
| 216 | 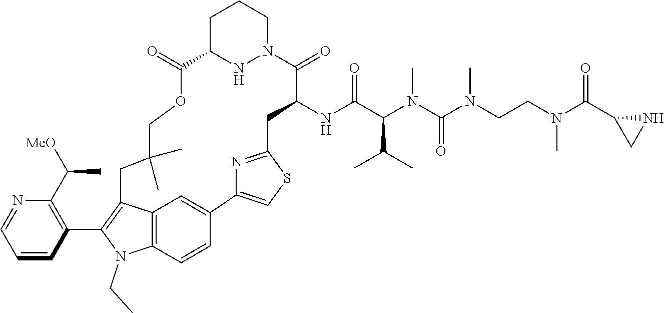 |
| 217 | 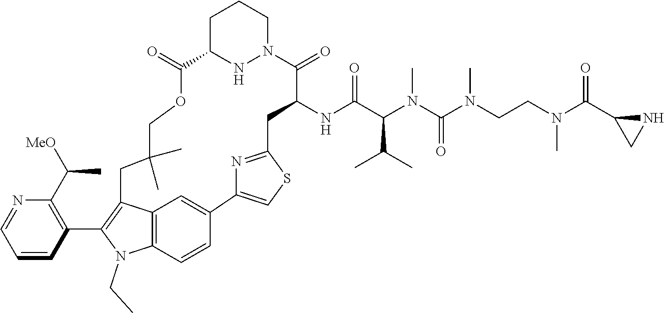 |
| 218 | 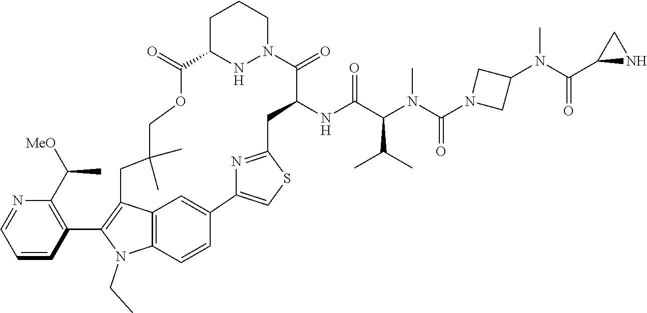 |
| 219 | 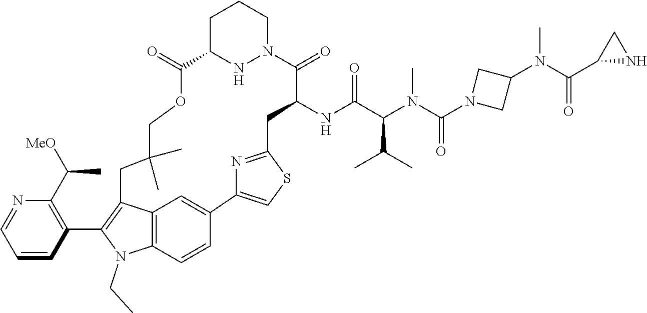 |
| 220 | 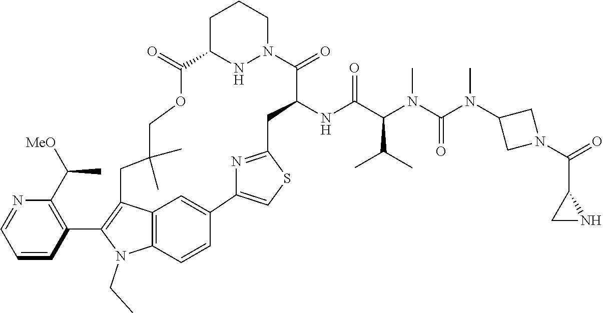 |
| 221 | 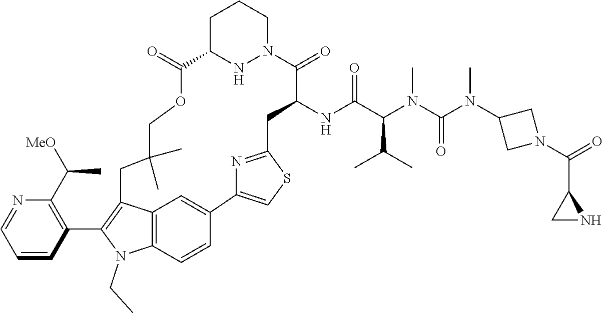 |
| 222 | 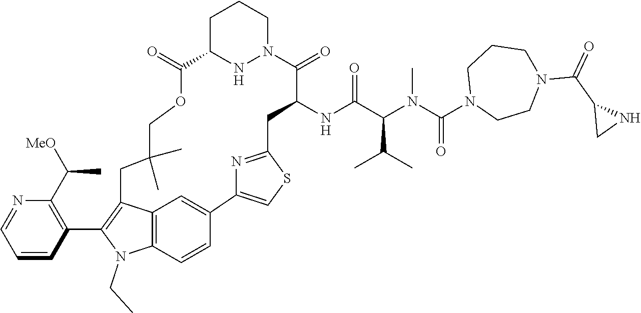 |
| 223 | 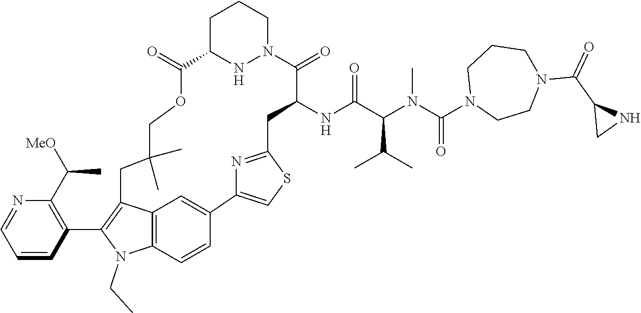 |
| 224 | 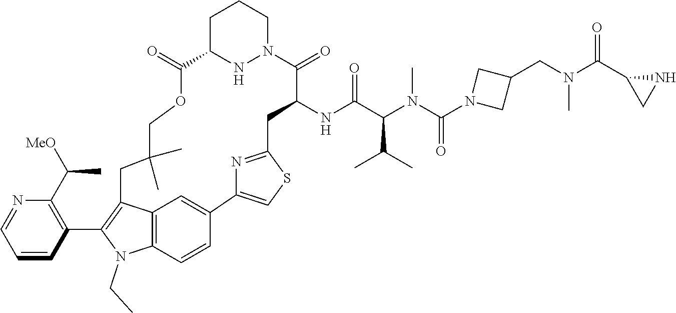 |
| 225 | 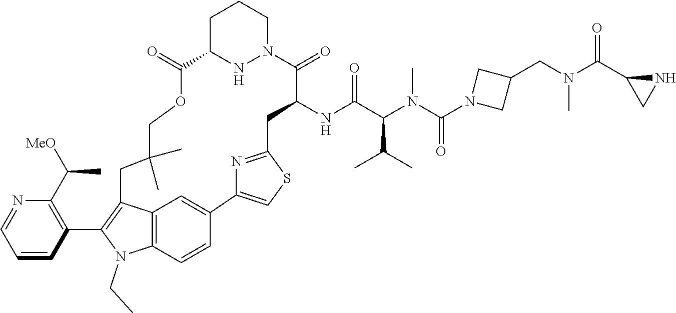 |
| 226 | 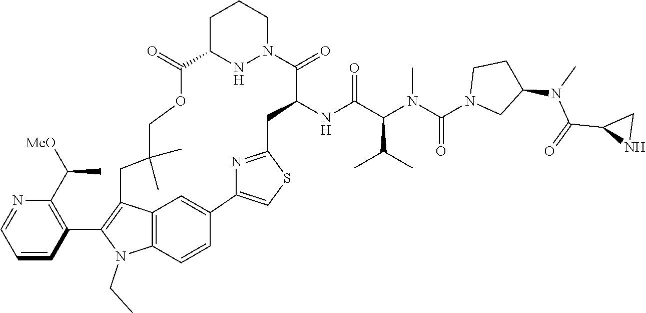 |
| 227 | 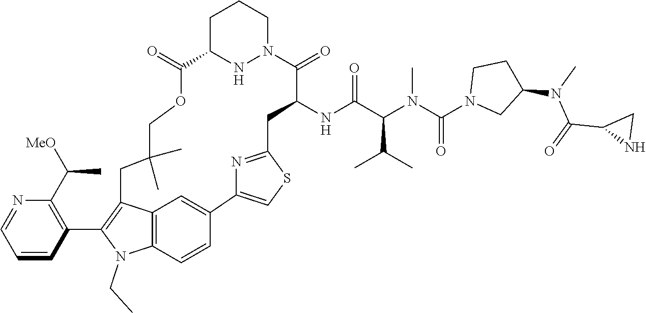 |
| 228 | 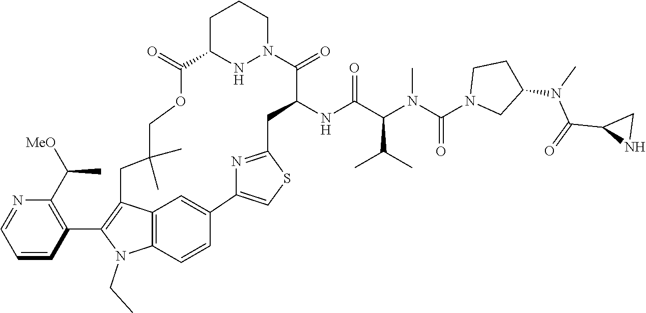 |
| 229 | 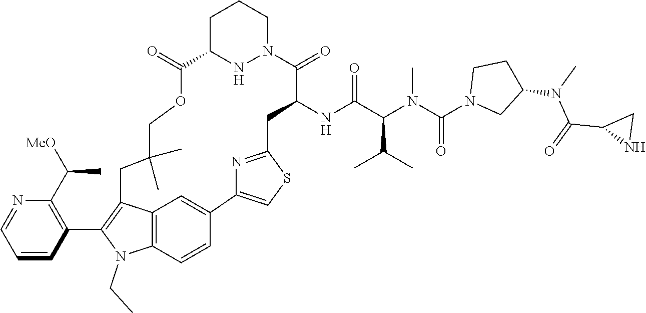 |
| 230 | 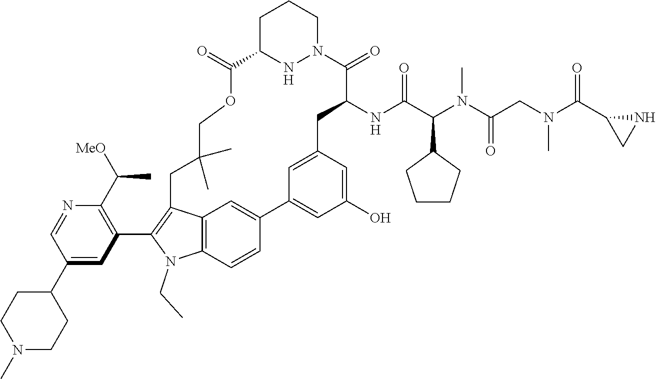 |
| 231 | 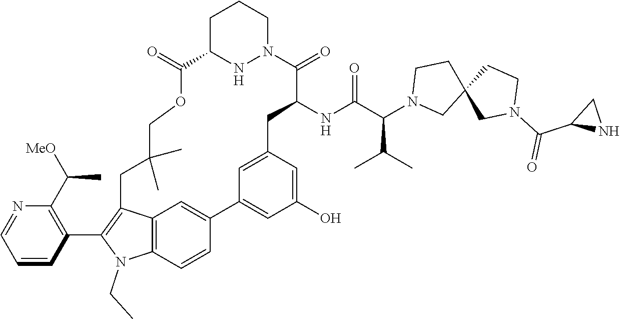 |
| 232 | 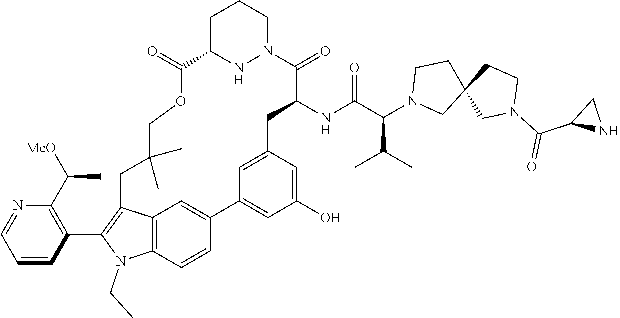 |
| 233 | 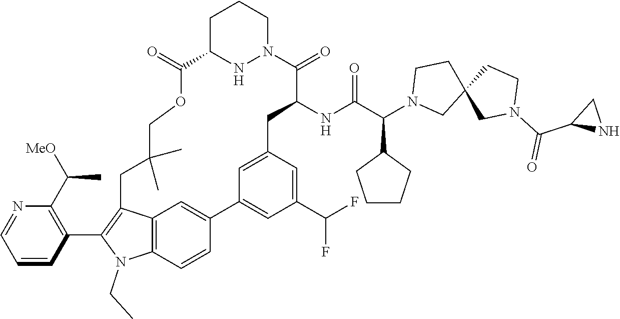 |
| 234 | 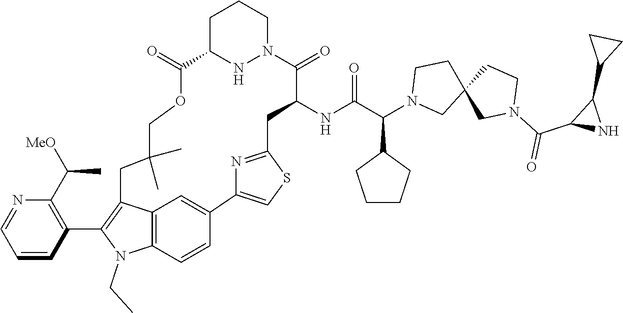 |
| 235 | 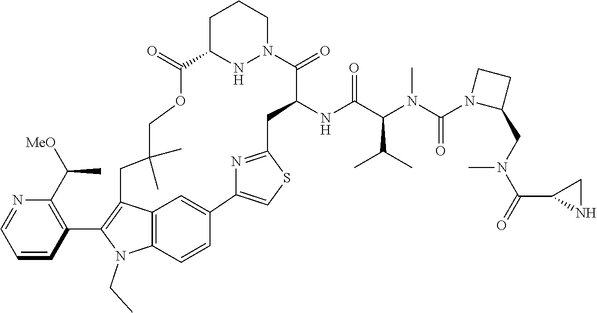 |
| 236 | 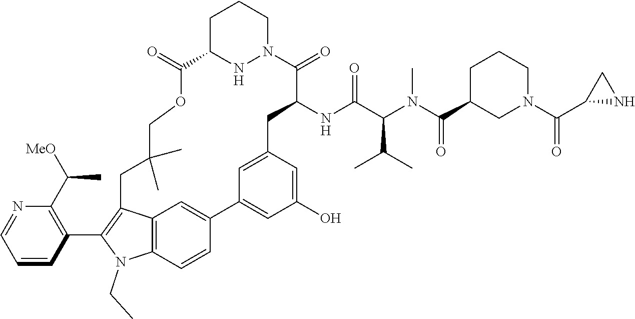 |
| 237 | 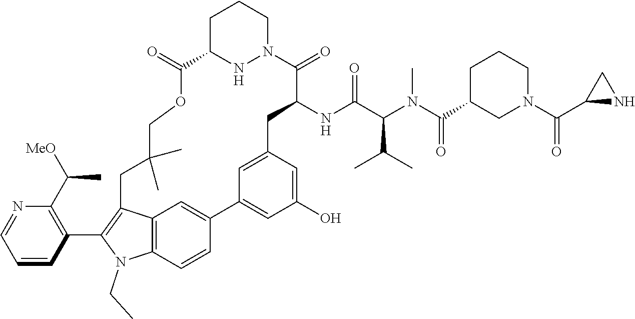 |
| 238 | 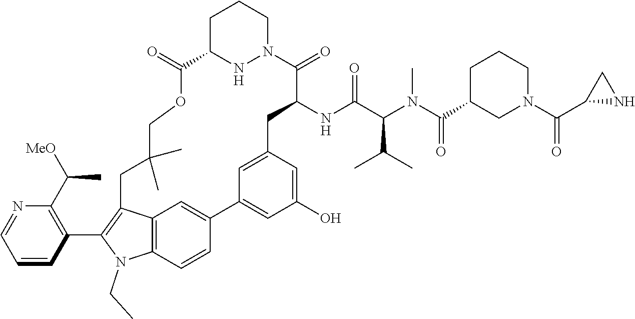 |
| 239 | 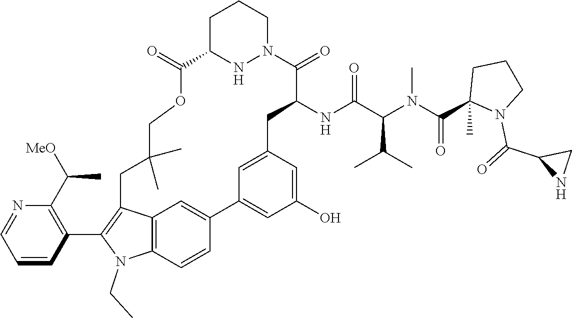 |
| 240 | 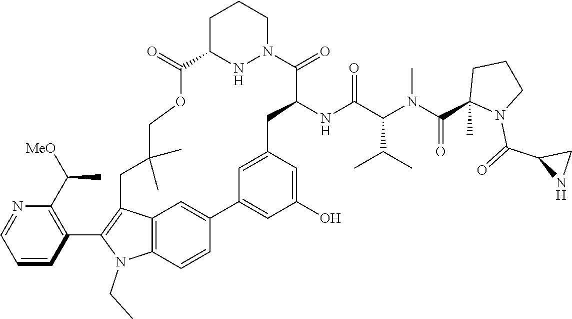 |
| 241 | 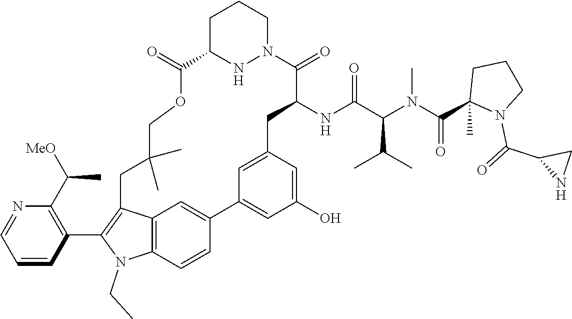 |
| 242 | 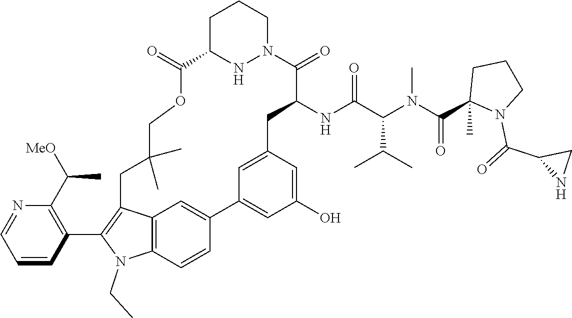 |
| 243 | 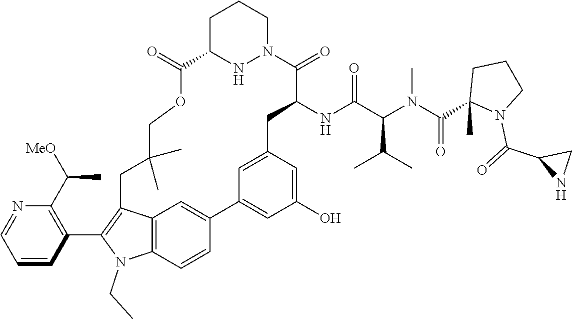 |
| 244 | 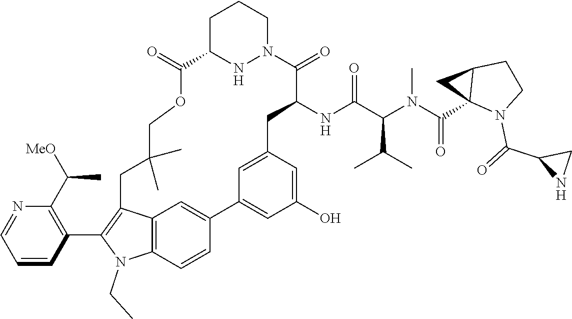 |
| 245 | 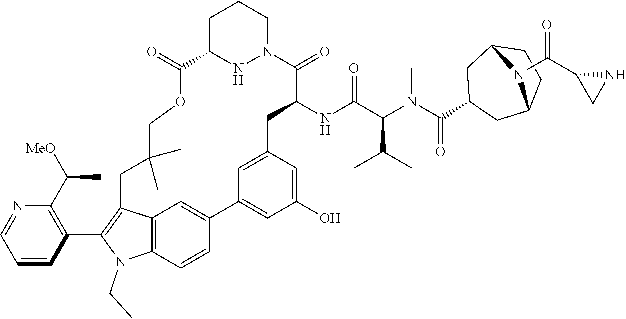 |
| 246 | 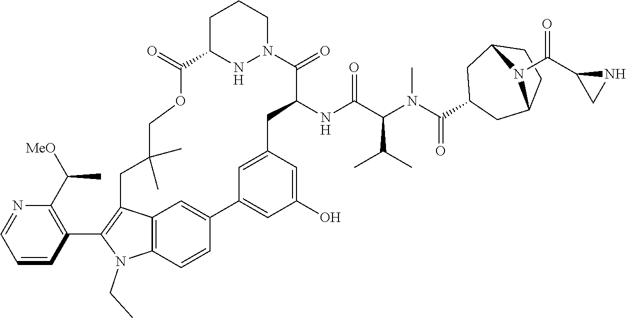 |
| 247 | 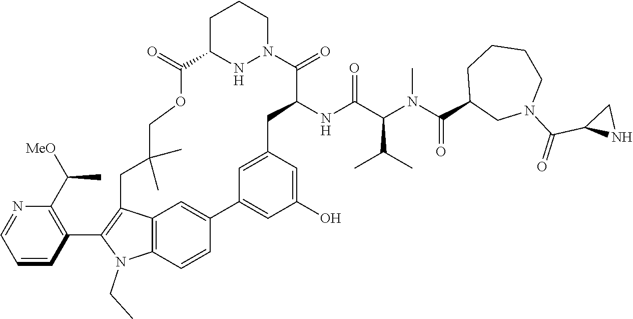 |
| 248 | 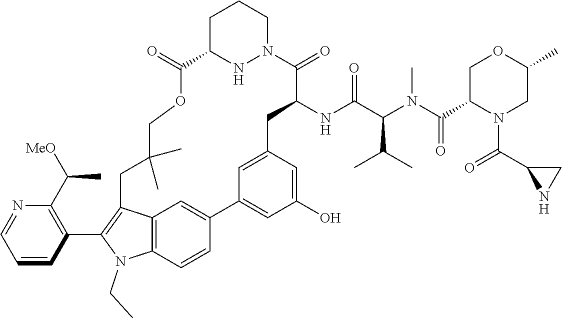 |
| 249 | 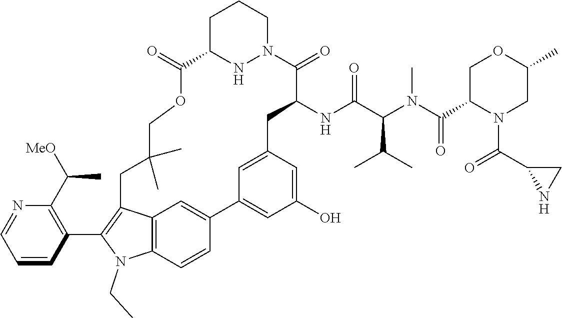 |
| 250 | 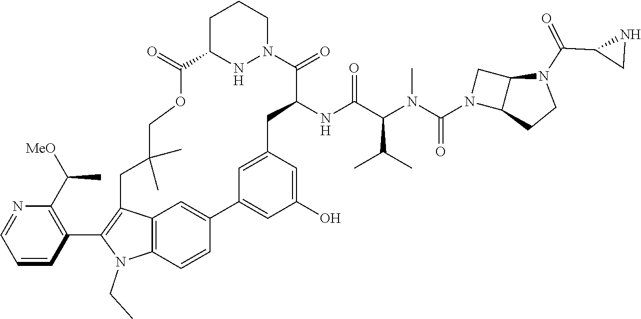 |
| 251 | 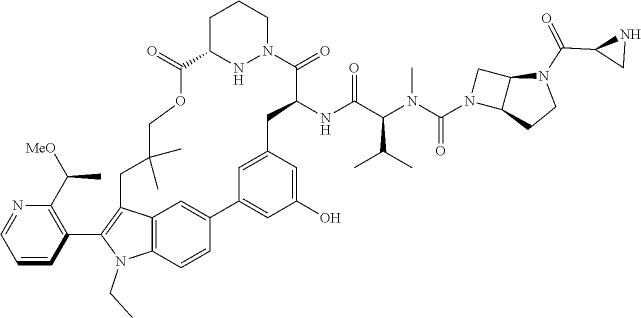 |
| 252 | 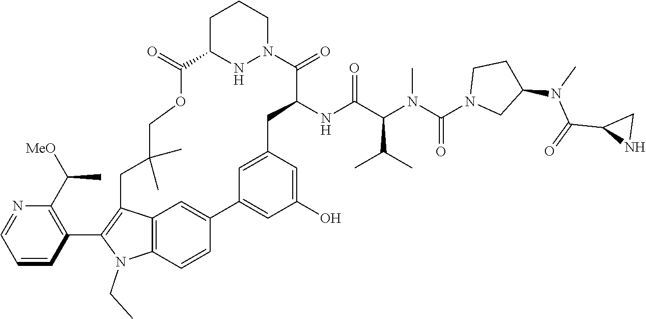 |
| 253 | 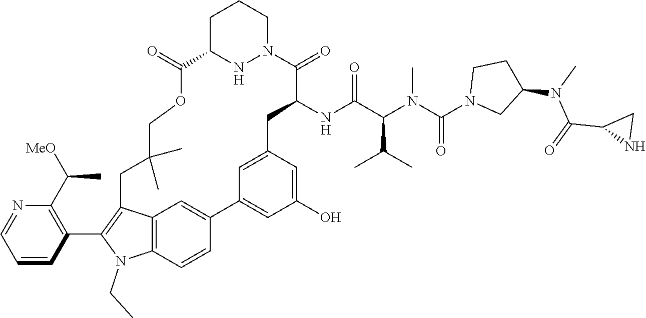 |
| 254 | 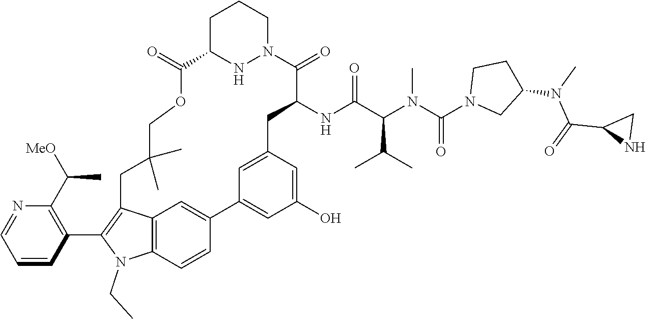 |
| 255 | 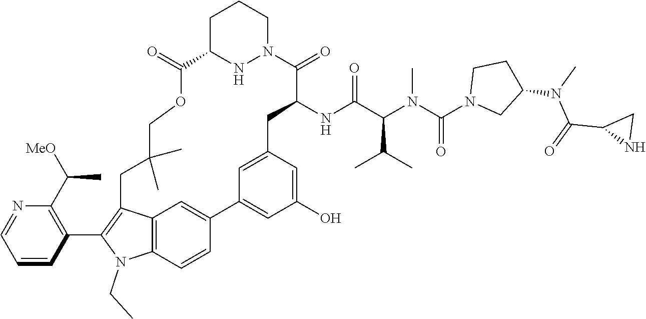 |
| 256 | 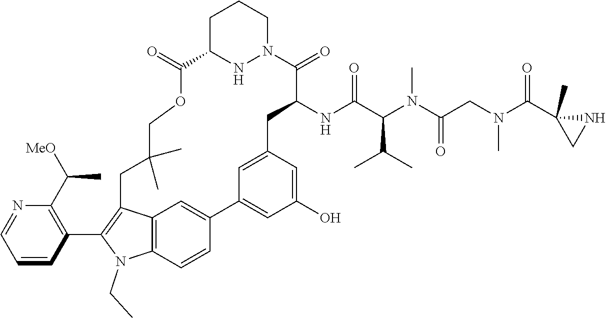 |
| 257 | 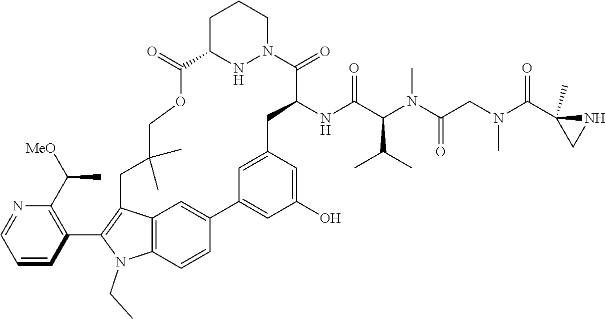 |
| 258* | 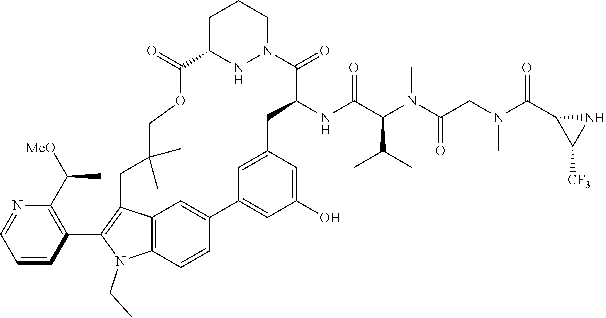 |
| 259* | 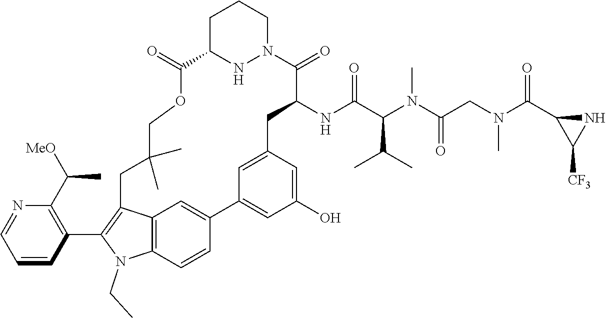 |
| 260* | 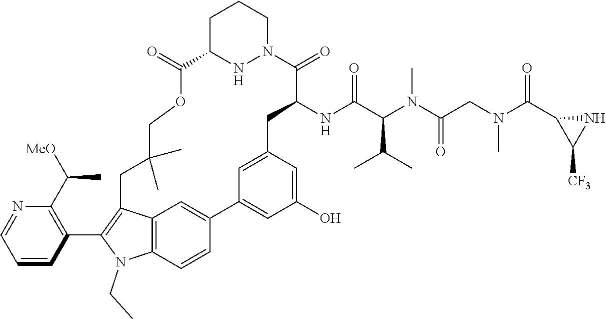 |
| 261* | 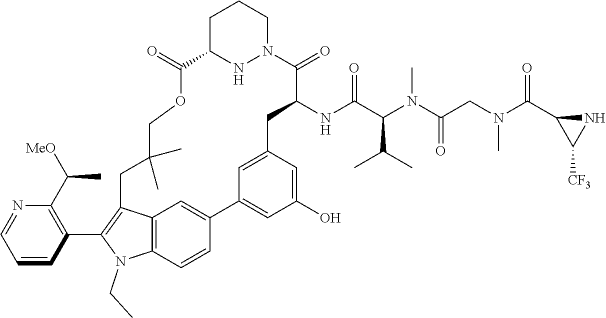 |
| 262 | 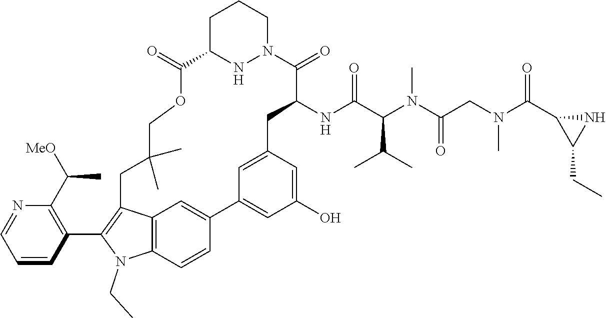 |
| 263 | 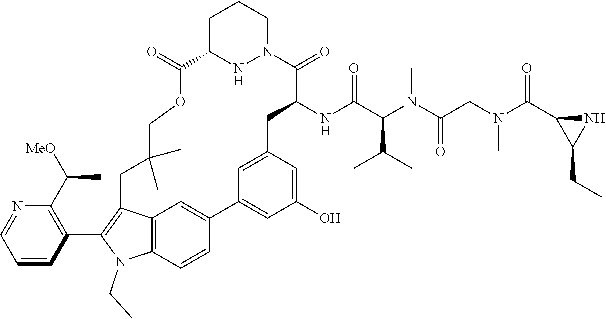 |
| 264 | 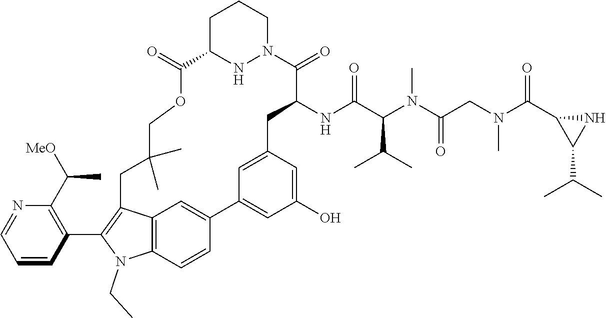 |
| 265 | 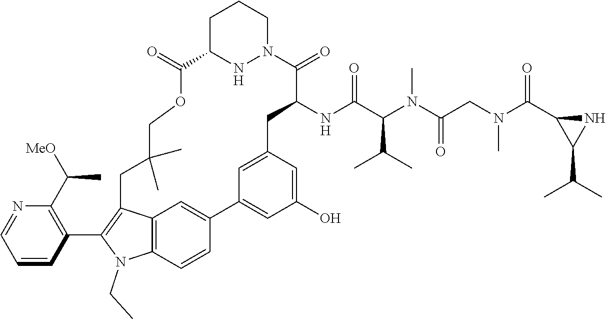 |
| 266 | 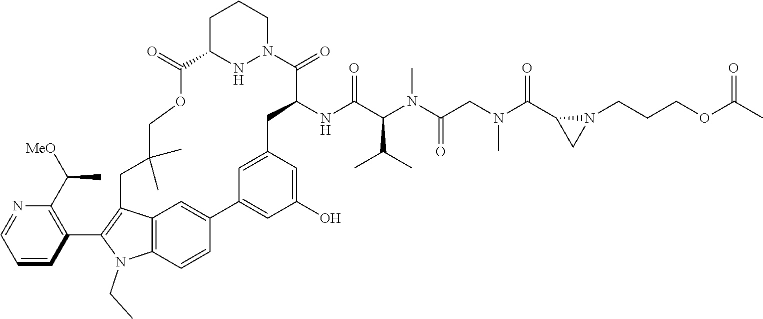 |
| 267 | 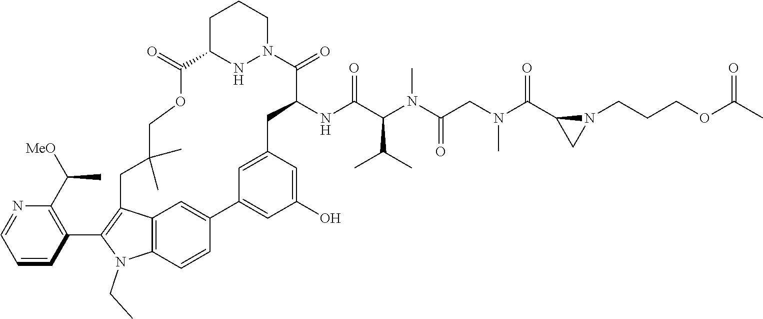 |
| 268 | 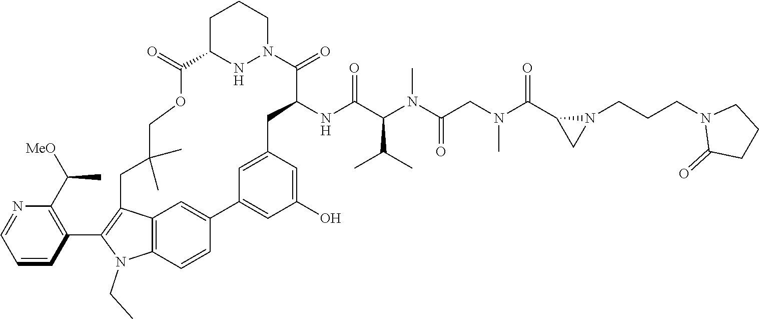 |
| 269 | 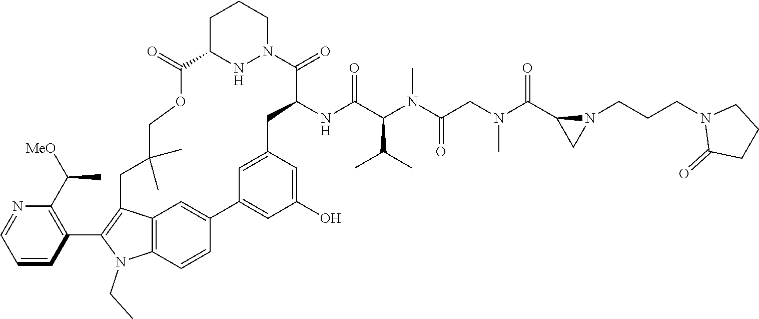 |
| 270 | 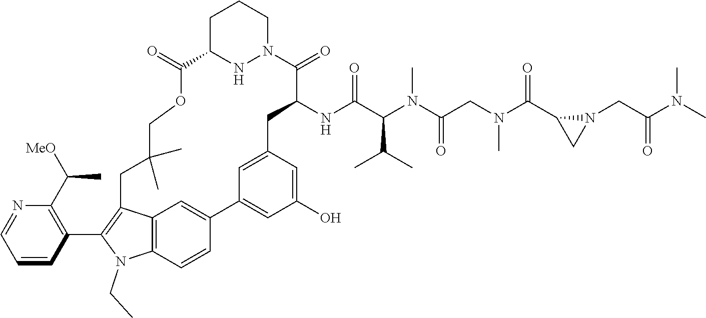 |
| 271 | 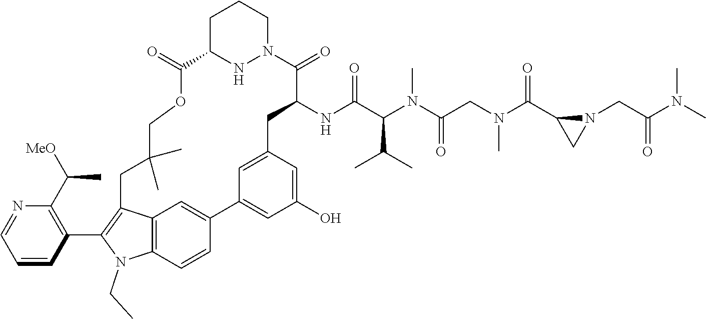 |
| 272 | 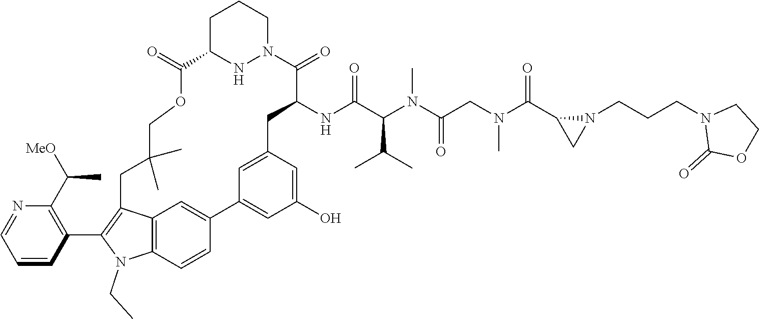 |
| 273 | 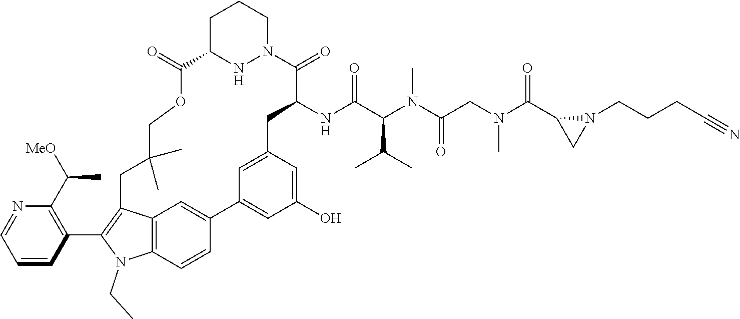 |
| 274 | 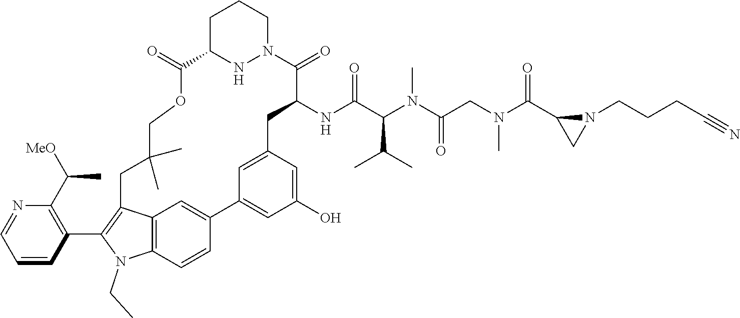 |
| 275 | 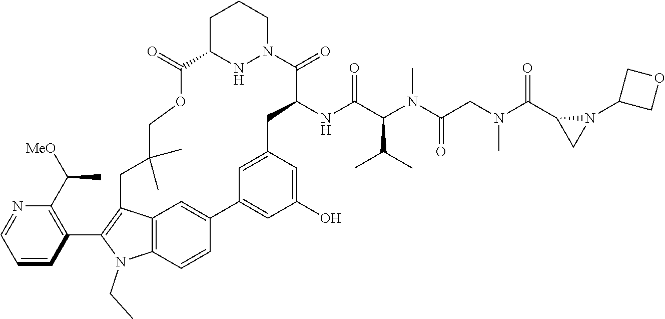 |
| 276 | 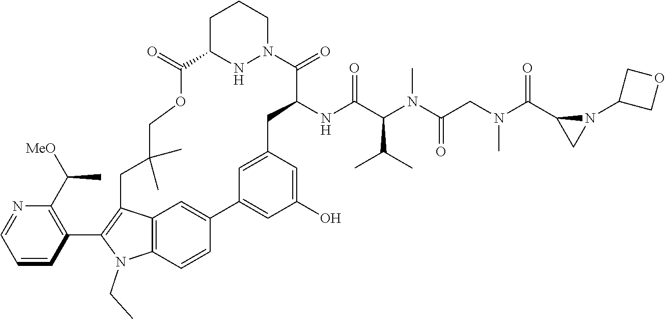 |
| 277 | 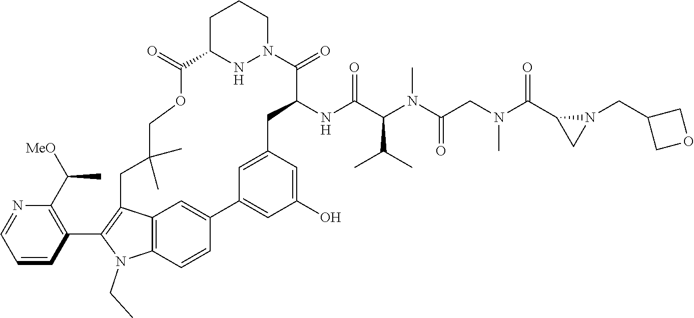 |
| 278 | 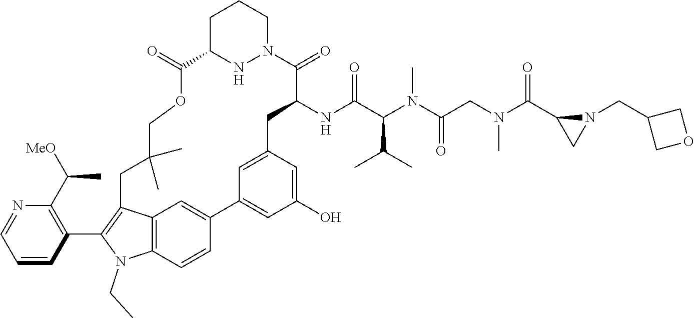 |
| 279 | 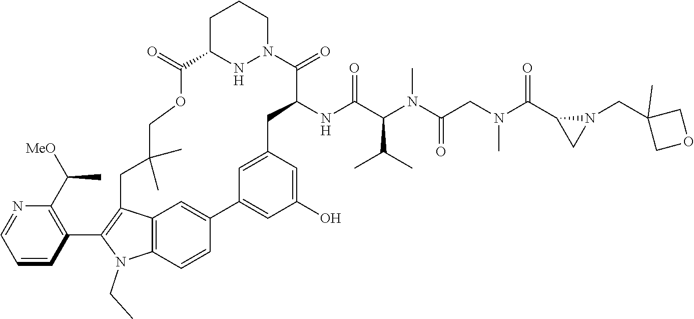 |
| 280 | 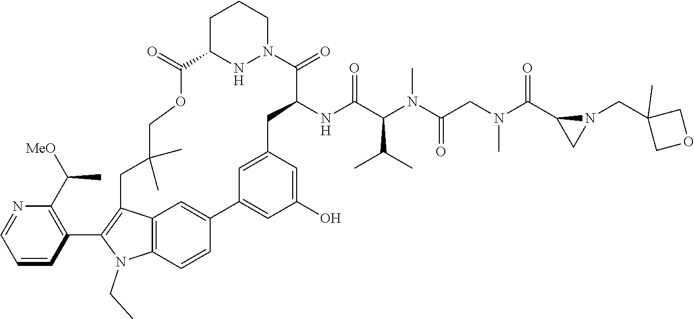 |
| 281* | 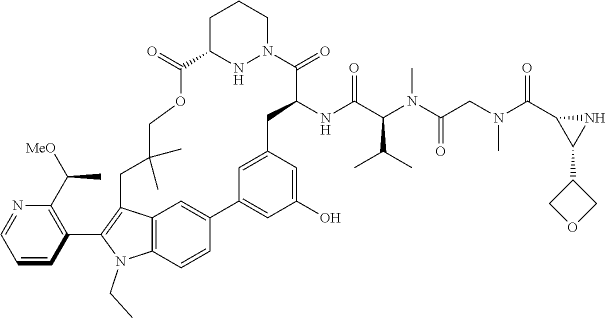 |
| 282* | 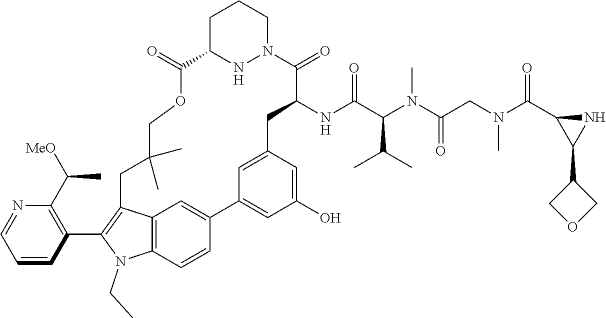 |
| 283* | 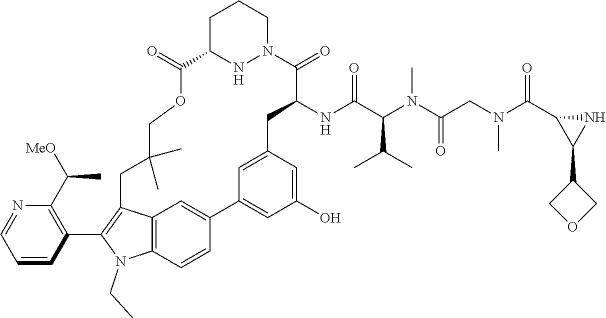 |
| 284* | 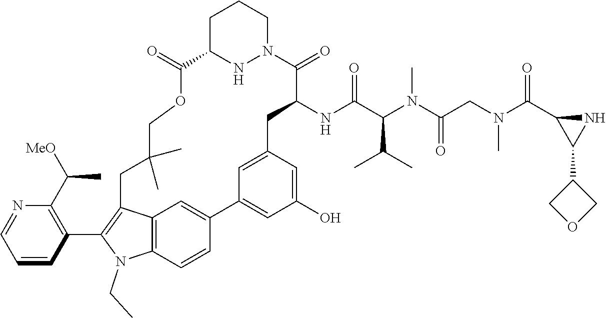 |
| 285 | 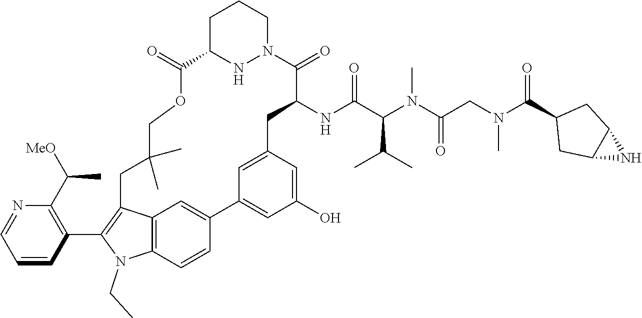 |
| 286 | 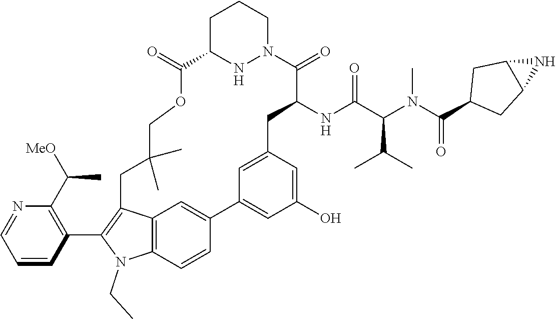 |
| 287 | 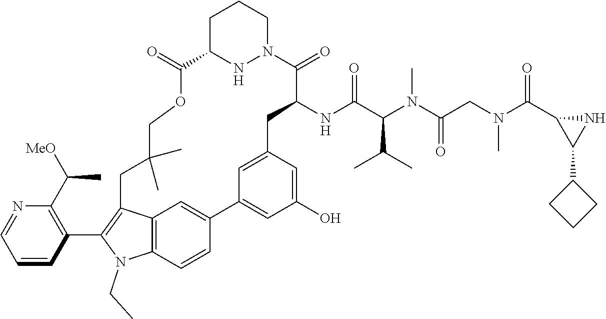 |
| 288 | 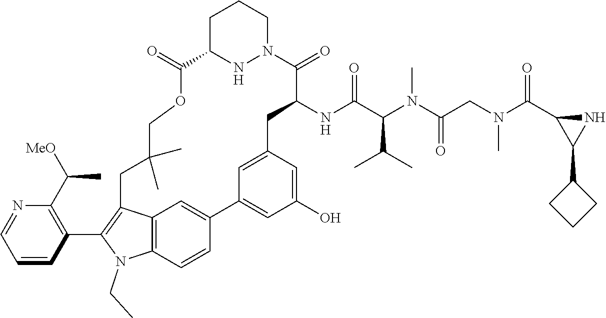 |
| 289 | 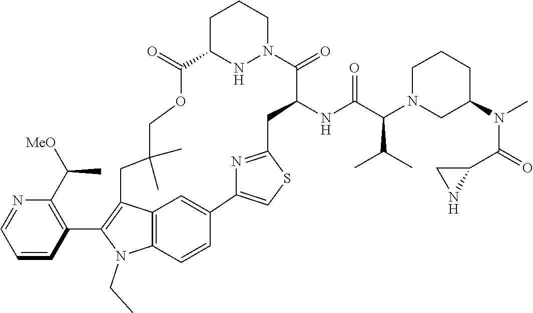 |
| 290 | 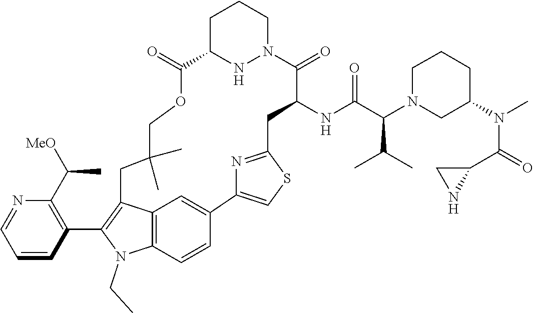 |
| 291 | 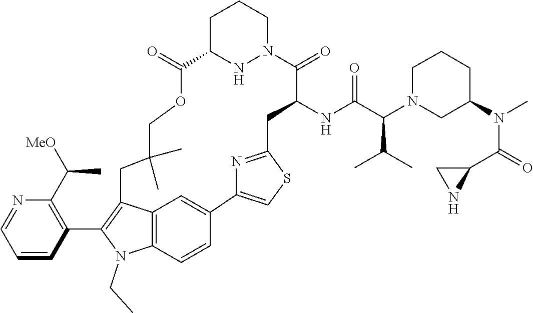 |
| 292 | 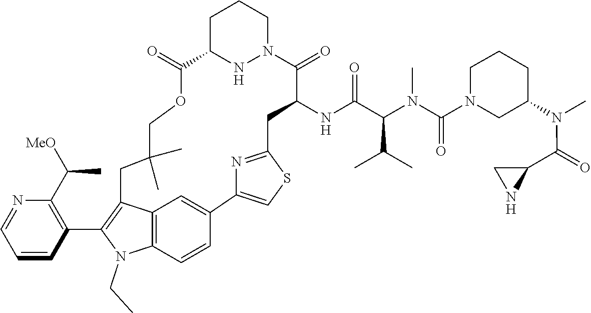 |
| 293 | 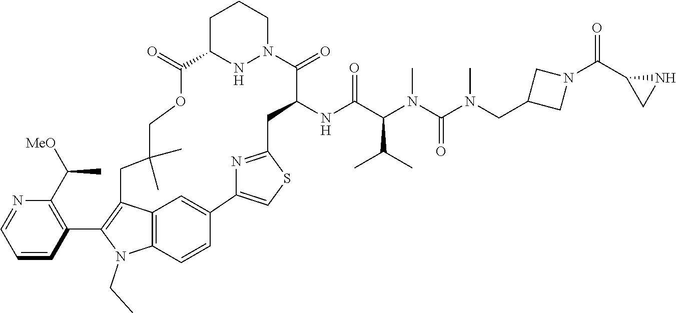 |
| 294 | 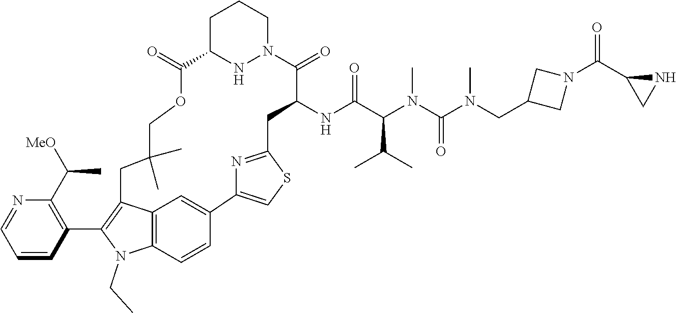 |
| 295 | 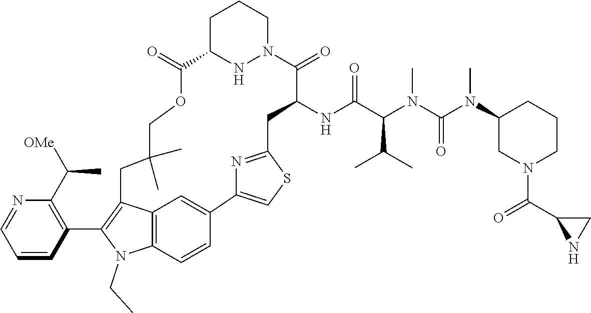 |
| 296 | 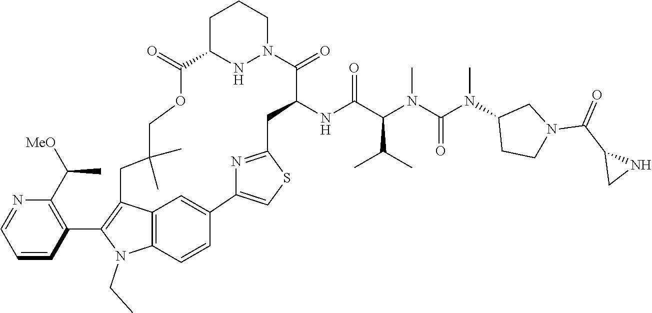 |
| 297 | 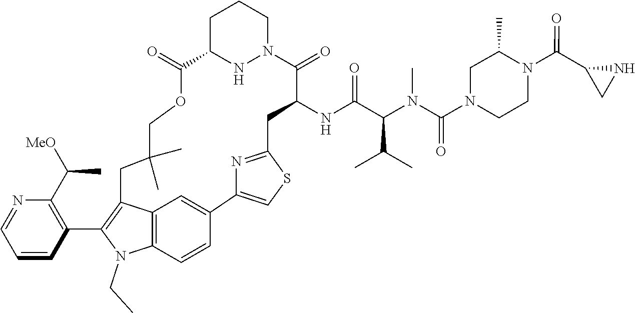 |
| 298 | 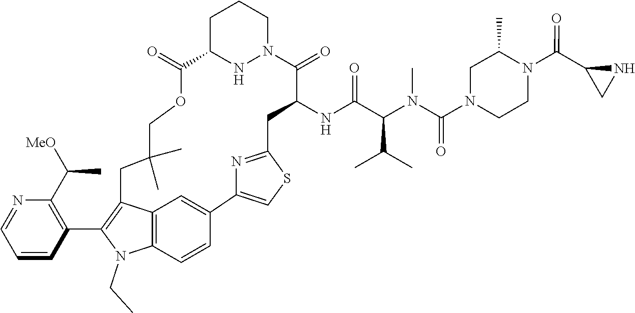 |
| 299 | 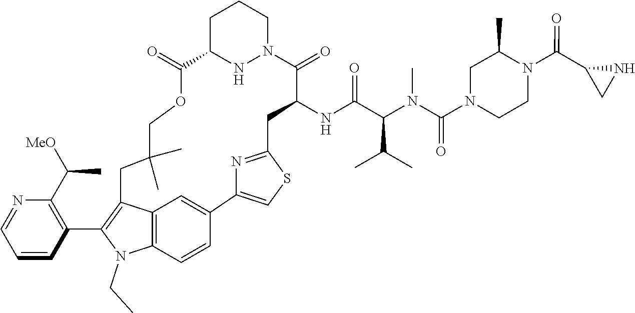 |
| 300 | 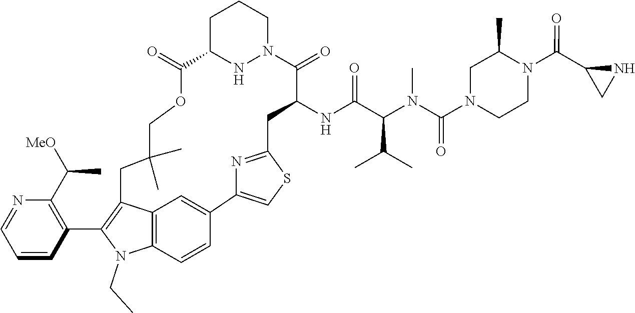 |
| 301 | 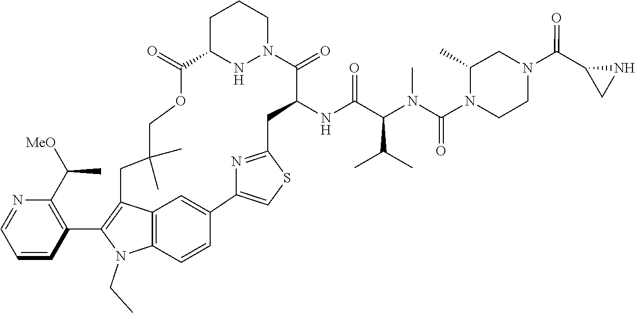 |
| 302 | 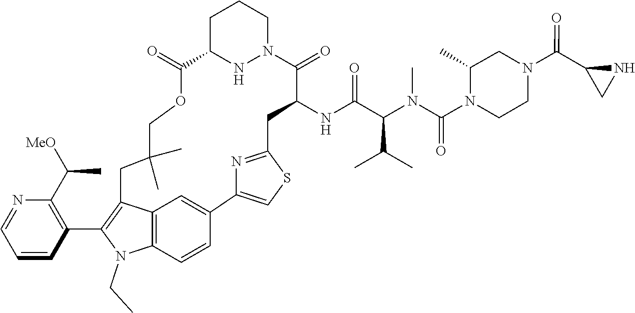 |
| 303 | 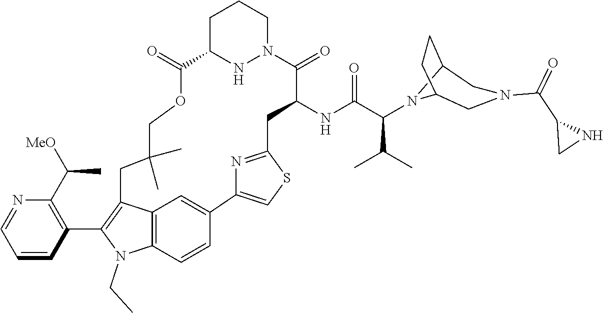 |
| 304 | 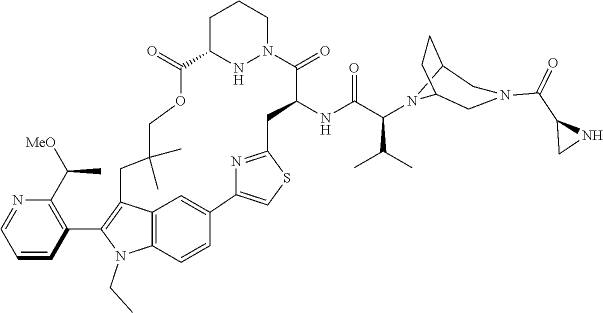 |
| 305 | 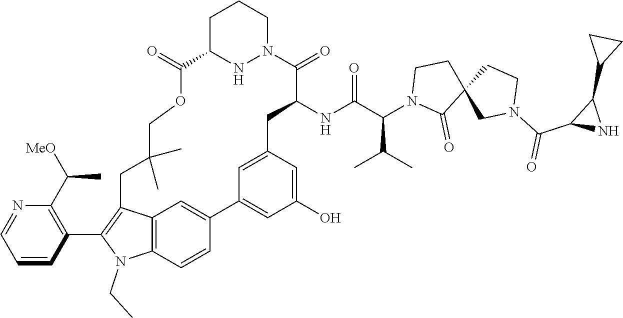 |
| 306 | 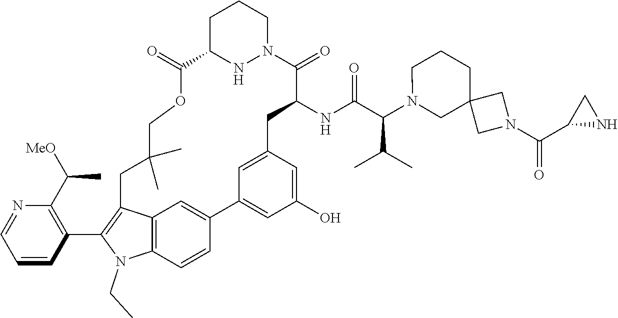 |
| 307 | 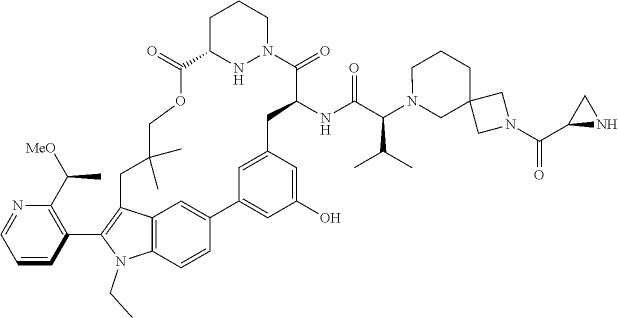 |
| 308* | 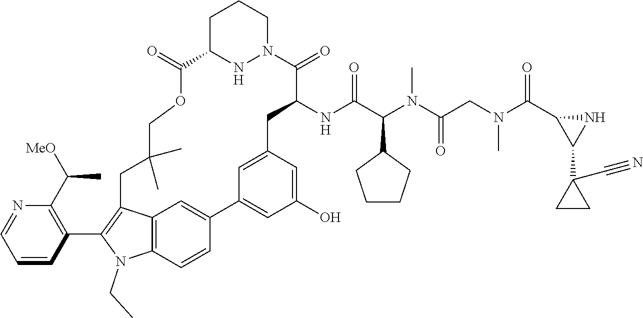 |
| 309 | 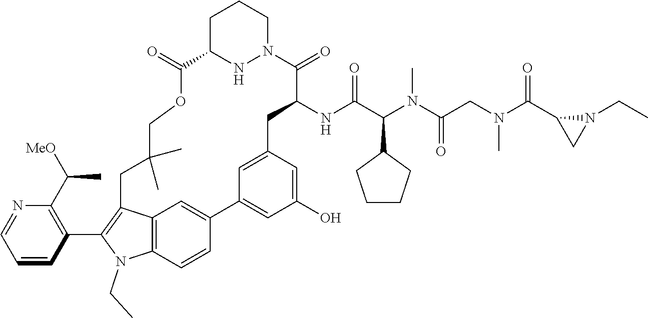 |
| 310 | 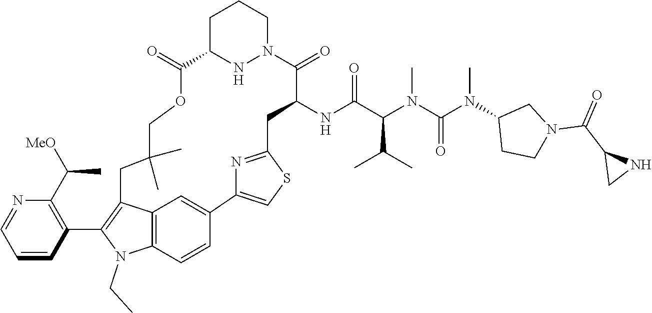 |
| 311 | 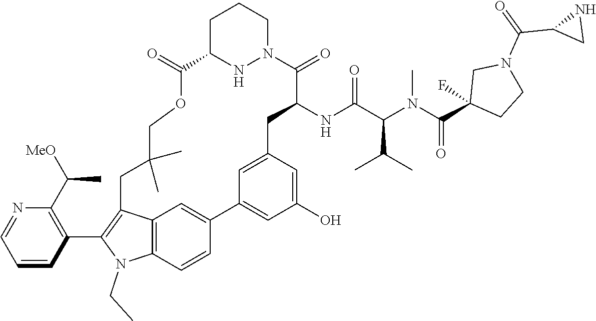 |
| 312 | 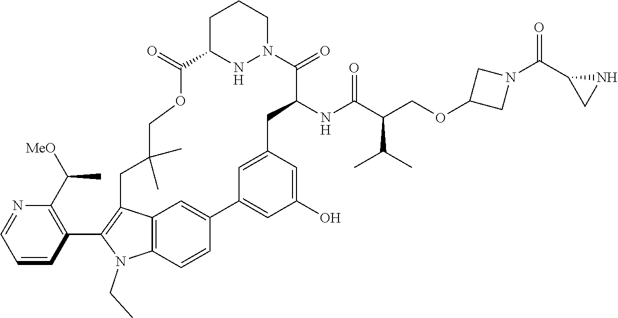 |
| 313 | 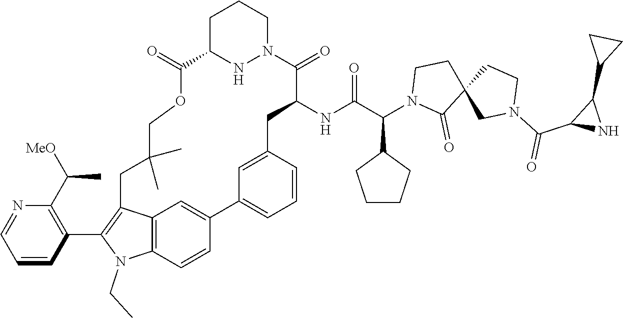 |
| 314* | 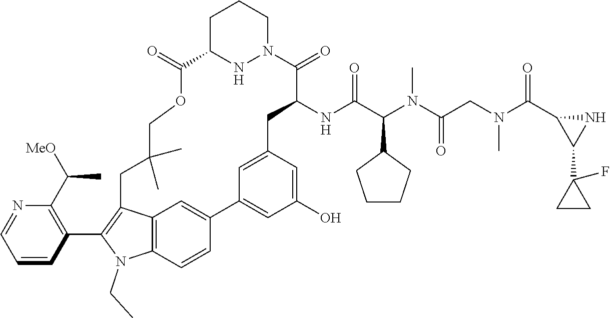 |
| 315* | 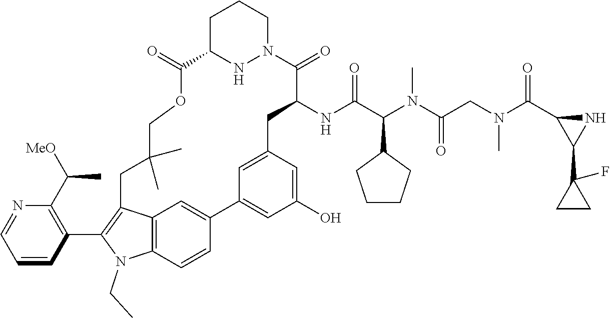 |
| 316* | 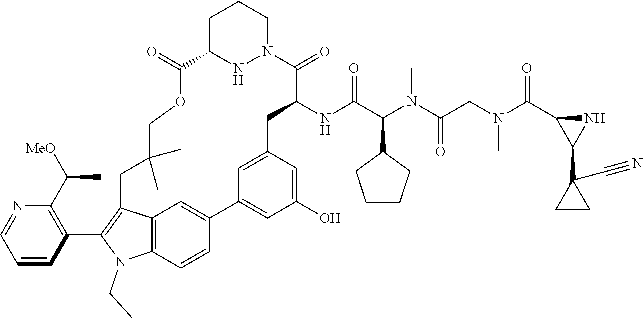 |
| 317 | 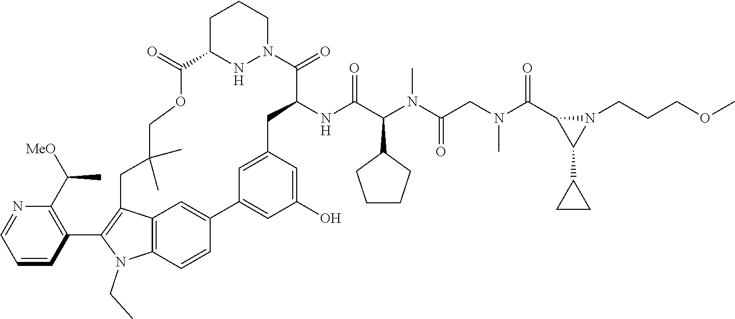 |
| 318 | 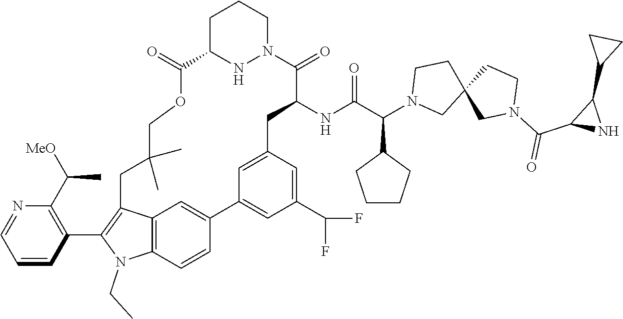 |
| 319 | 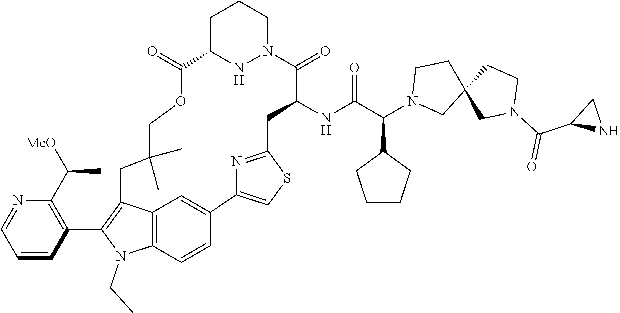 |
| 320 | 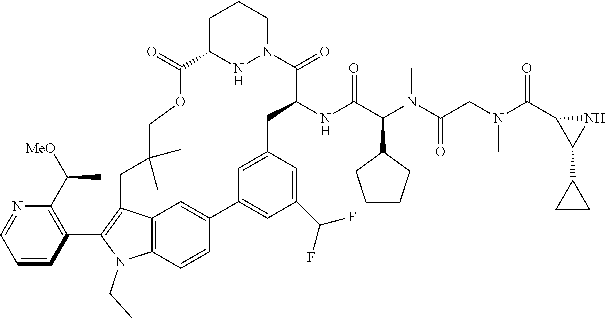 |
| 321 | 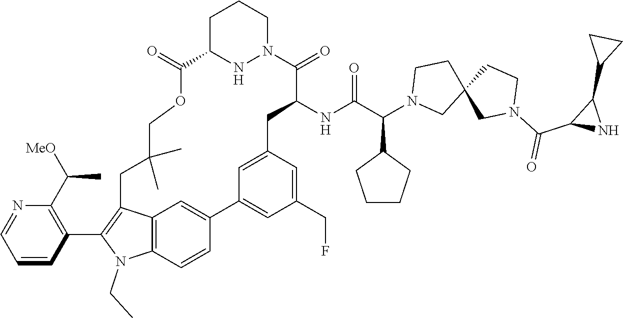 |
| 322* | 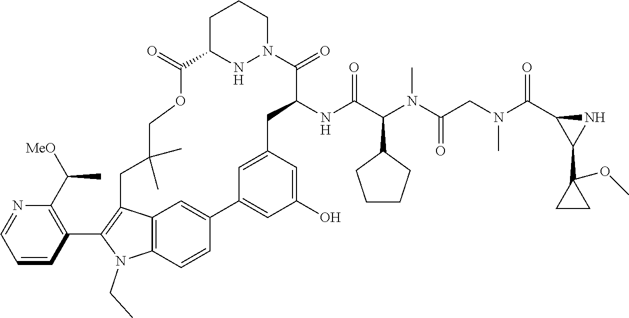 |
| 323* | 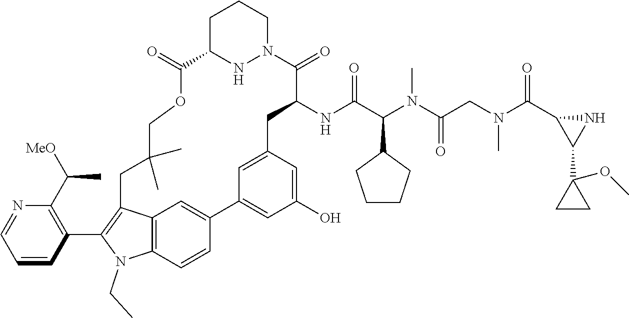 |
| 324 | 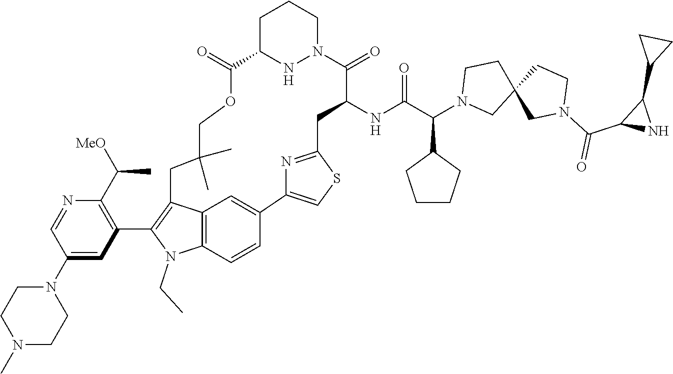 |
| 325 | 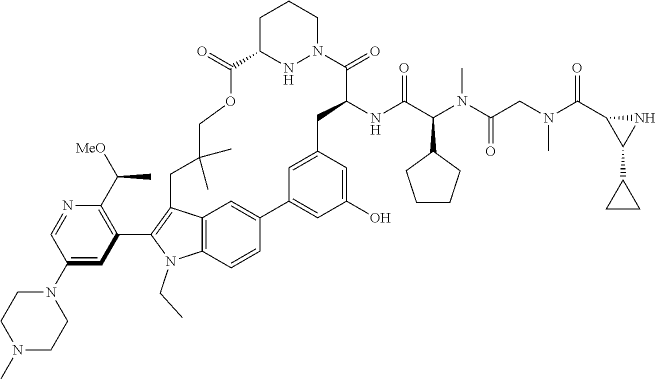 |
| 326 | 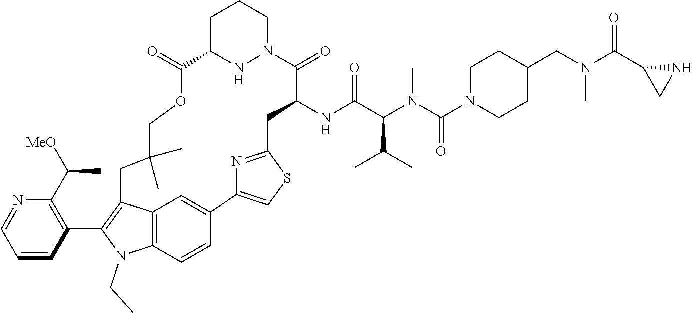 |
| 327 | 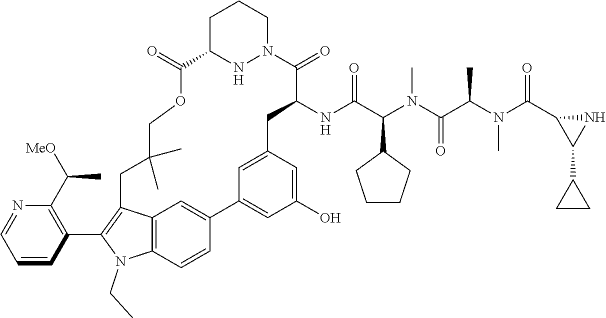 |
| 328 | 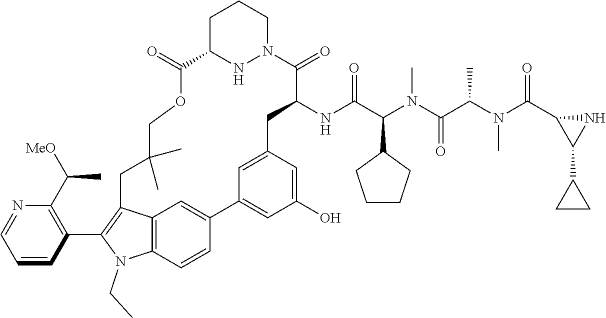 |
| 329 | 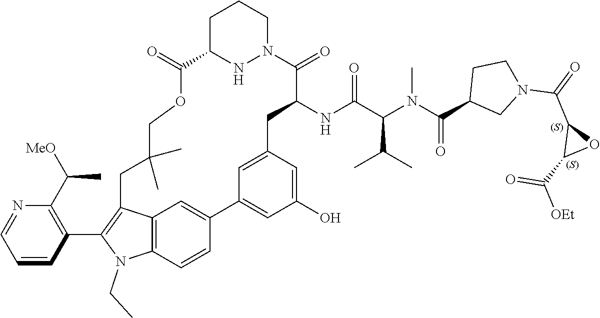 |
| 330 | 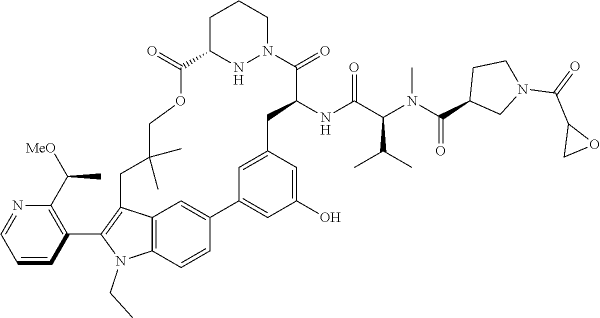 |
| 331 | 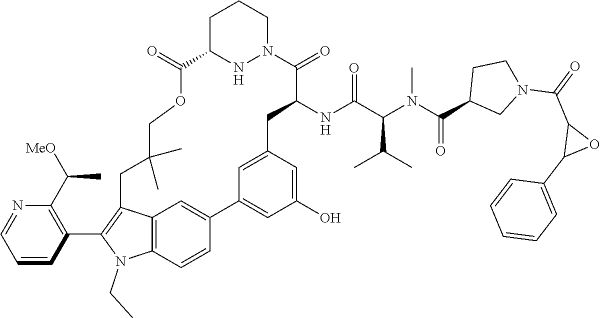 |
| 332 | 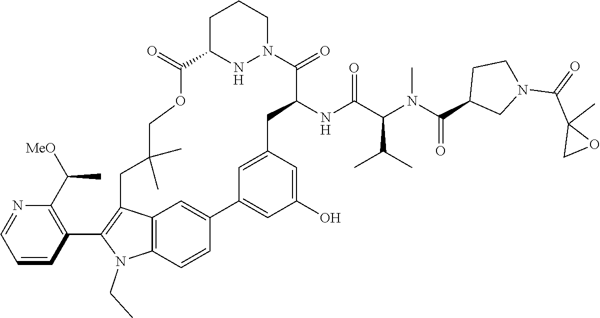 |

* Stereochemistry of the aziridine carbon is assumed.
Note that some compounds are shown with bonds as fiat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments, a compound of Table 2 is provided, or a pharmaceutically acceptable salt thereof. In some embodiments, a compound of the present invention is selected from Table 2, or a pharmaceutically acceptable salt or atropisomer thereof.

TABLE 2
Certain Compounds of the Present Invention

| Ex# | Structure |
|---|---|
| B5 | 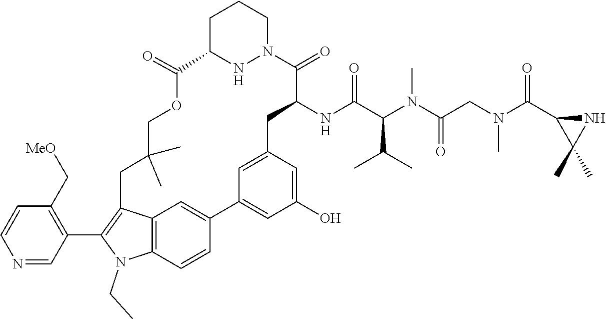 |
| B6 | 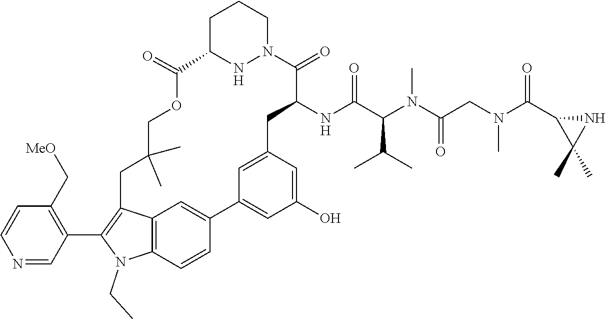 |
| B8 | 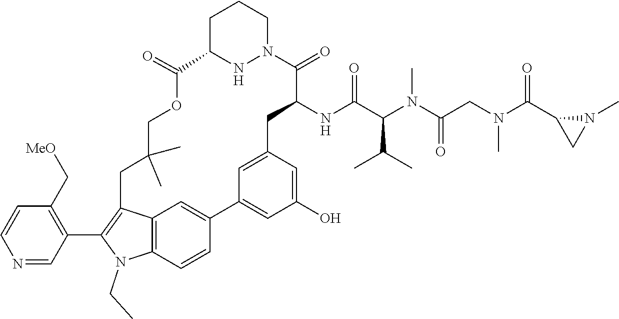 |
| B9 | 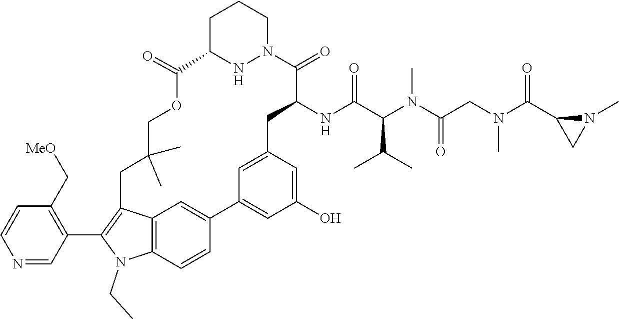 |
| B16 | 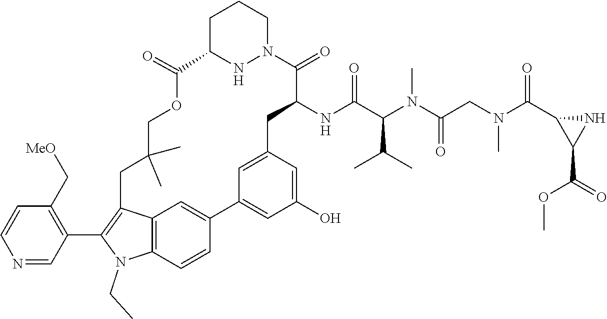 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C(=O)OC)C(=O)N1CCC[C@H](N1)C(=O)OCC(C)(C)C2] |
| B19 | 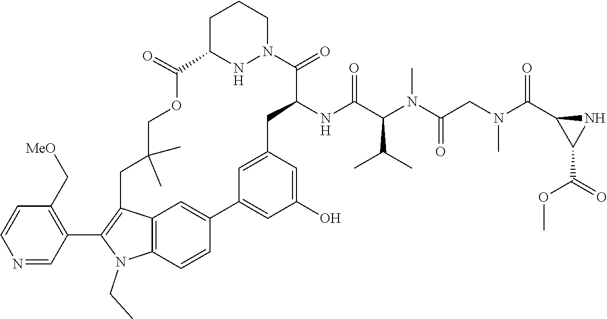 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C(=O)OC)C(=O)N1CCC[C@H](N1)C(=O)OCC(C)(C)C2] |
| B29 | 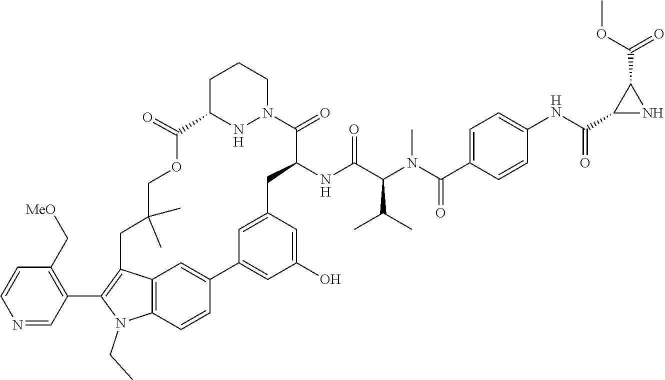 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@H]3C(=O)OC)cc1)C(=O)N1CCC[C@H](N1)C(=O)OCC(C)(C)C2] |
| B30 | 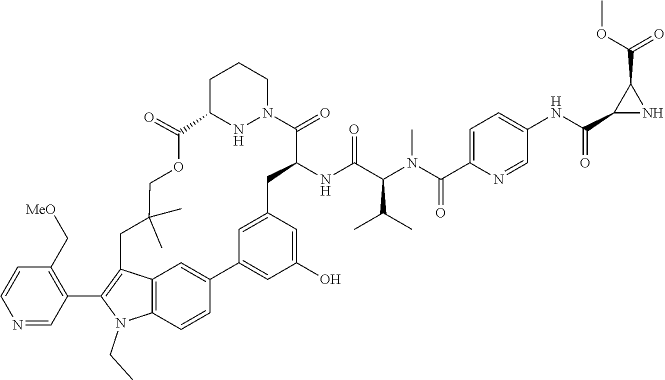 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@@H]3C(=O)OC)cn1)C(=O)N1CCC[C@H](N1)C(=O)OCC(C)(C)C2] |
| B31 | 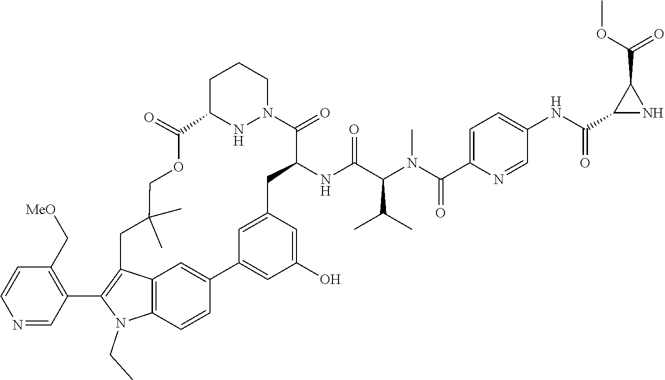 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@@H]3C(=O)OC)cn1)C(=O)N1CCC[C@H](N1)C(=O)OCC(C)(C)C2] |
| B32 | 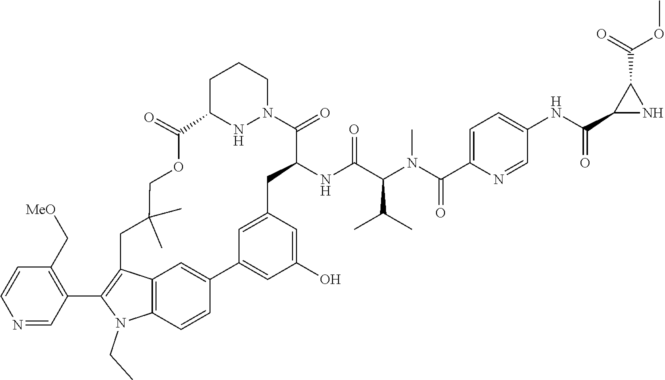 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@H]3C(=O)OC)cn1)C(=O)N1CCC[C@H](N1)C(=O)OCC(C)(C)C2] |
| B35 | 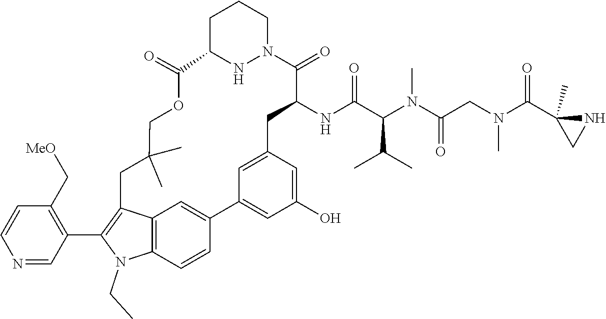 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@]1(C)CN1)C(=O)N1CCC[C@H](N1)C(=O)OCC(C)(C)C2] |
| B36 | 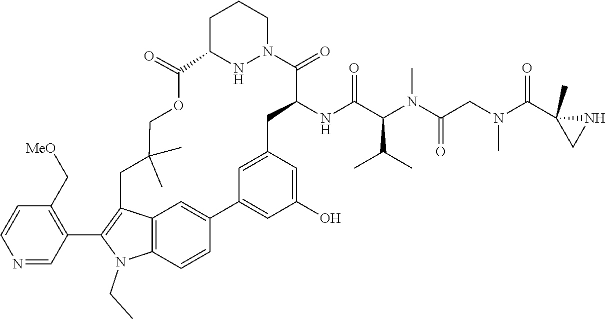 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@]1(C)CN1)C(=O)N1CCC[C@H](N1)C(=O)OCC(C)(C)C2] |
| B37 | 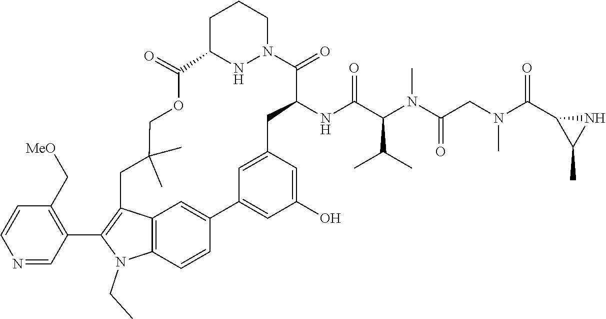 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C)C(=O)N1CCC[C@H](N1)C(=O)OCC(C)(C)C2] |
| B38 | 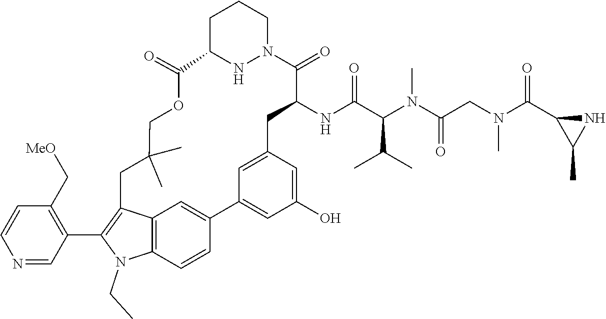 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C)C(=O)N1CCC[C@H](N1)C(=O)OCC(C)(C)C2] |
| B40 | 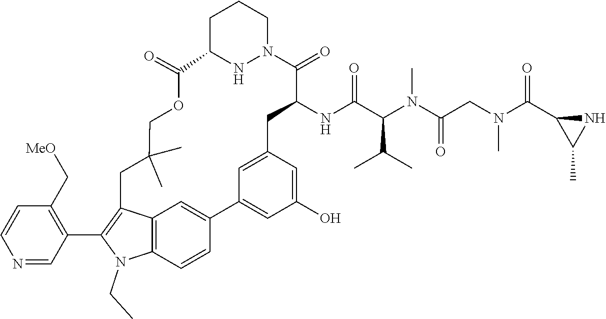 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C)C(=O)N1CCC[C@H](N1)C(=O)OCC(C)(C)C2] |
| B41 | 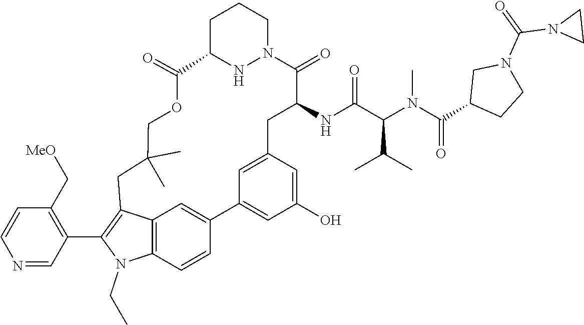 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3CC3)C1)C(=O)N1CCC[C@H](N1)C(=O)OCC(C)(C)C2] |
| B42 | 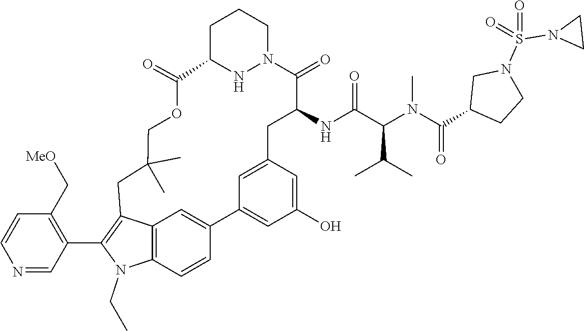 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3CC3)C1)C(=O)N1CCC[C@H](N1)C(=O)OCC(C)(C)C2] |
| B43 | 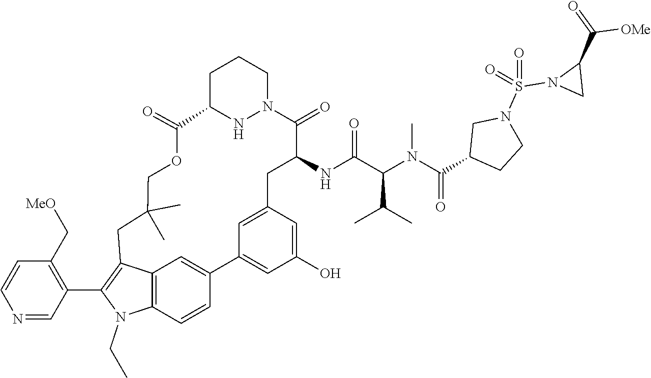 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@@H]3C(=O)OC)C1)C(=O)N1CCC[C@H](N1)C(=O)OCC(C)(C)C2] |
| B44 | 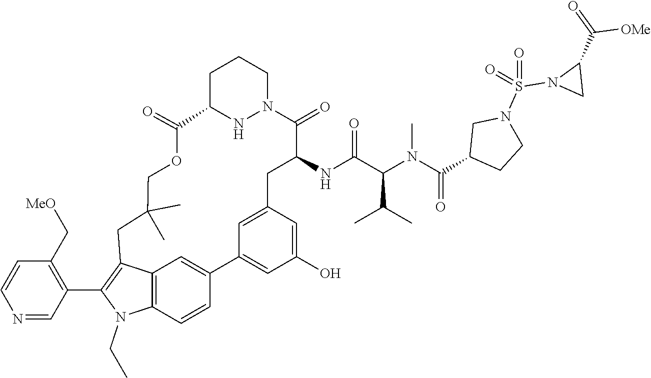 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@H]3C(=O)OC)C1)C(=O)N1CCC[C@H](N1)C(=O)OCC(C)(C)C2] |
| B46 | 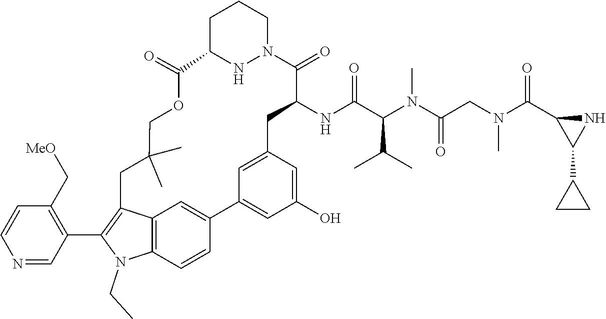 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C1CC1)C(=O)N1CCC[C@H](N1)C(=O)OCC(C)(C)C2] |
| B48 | 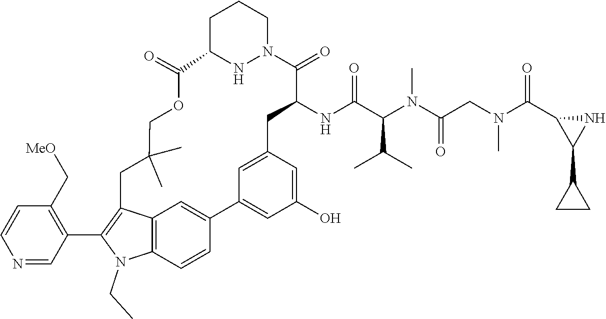 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C1CC1)C(=O)N1CCC[C@H](N1)C(=O)OCC(C)(C)C2] |
| B51 | 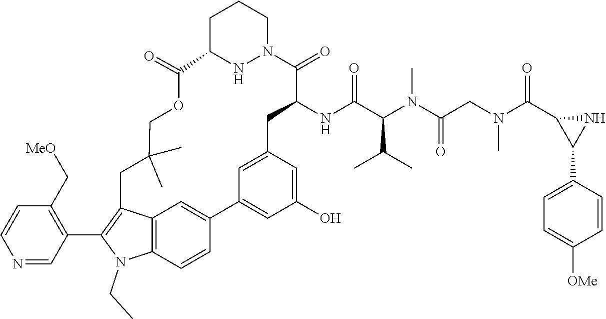 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccc(OC)cc1)C(=O)N1CCC[C@H](N1)C(=O)OCC(C)(C)C2] |
| B53 | 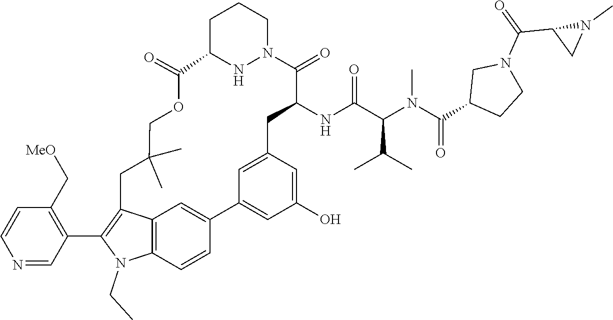 |
| B54 | 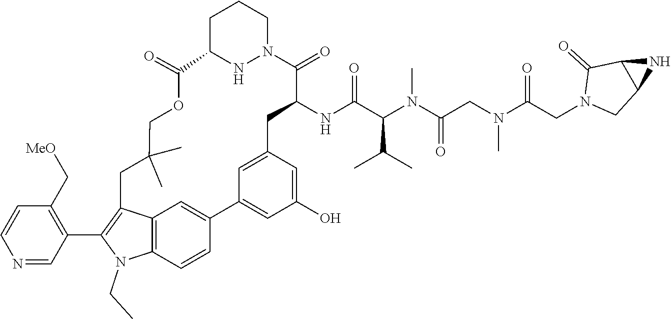 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@@H]3N[C@@H]3C1=O)C(=O)N1CCC[C@H](N1)C(=O)OCC(C)(C)C2] |
| B55 | 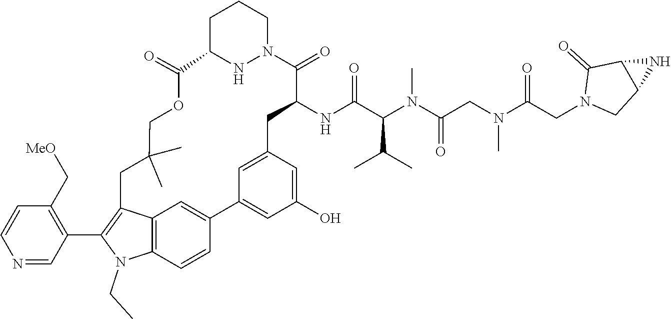 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@H]3N[C@H]3C1=O)C(=O)N1CCC[C@H](N1)C(=O)OCC(C)(C)C2] |
| B56 | 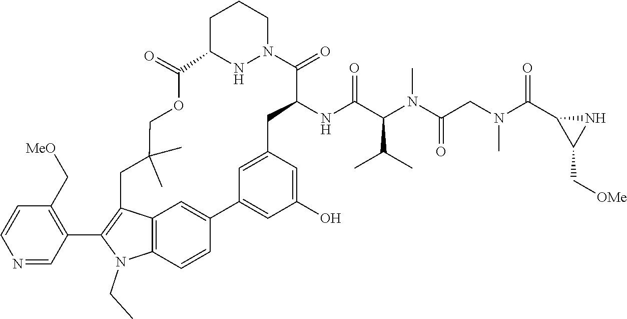 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1COC)C(=O)N1CCC[C@H](N1)C(=O)OCC(C)(C)C2] |
| B57 | 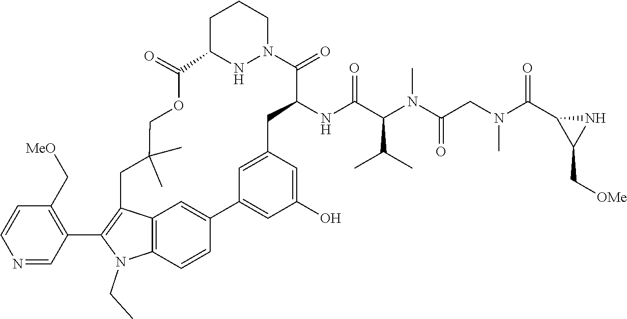 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1COC)C(=O)N1CCC[C@H](N1)C(=O)OCC(C)(C)C2] |
| B58 | 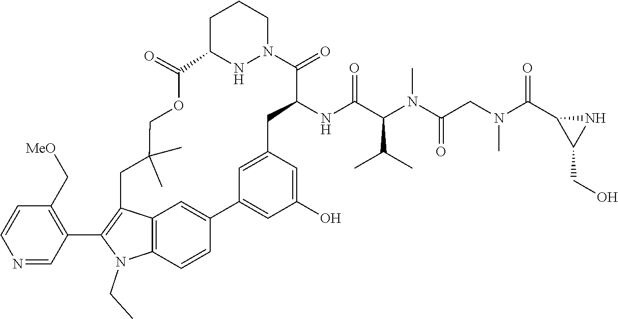 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1CO)C(=O)N1CCC[C@H](N1)C(=O)OCC(C)(C)C2] |
| B59 | 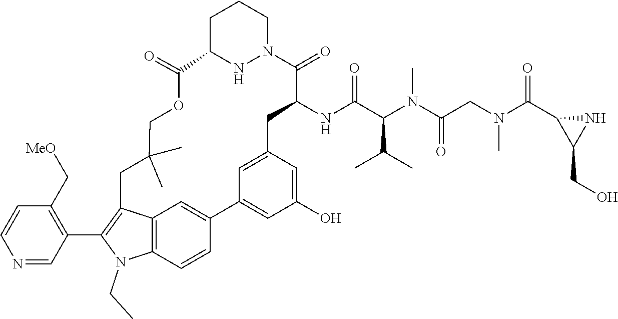 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1CO)C(=O)N1CCC[C@H](N1)C(=O)OCC(C)(C)C2] |
| B60 | 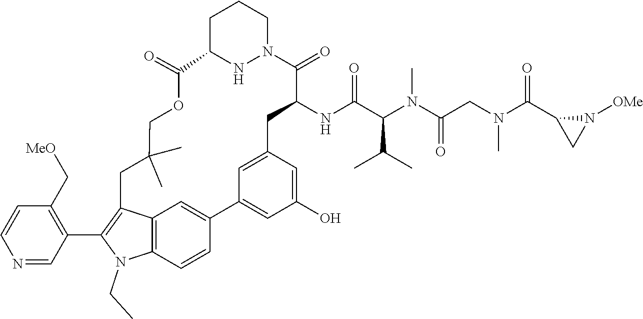 |
| B61 | 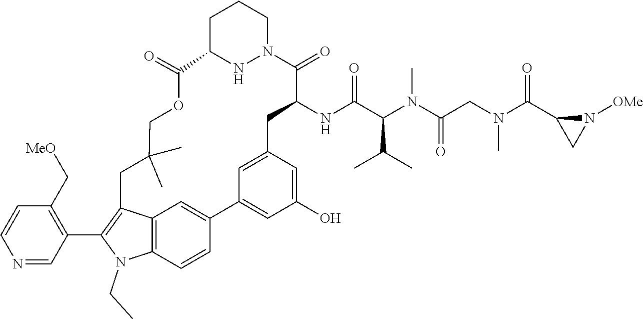 |
| B62 | 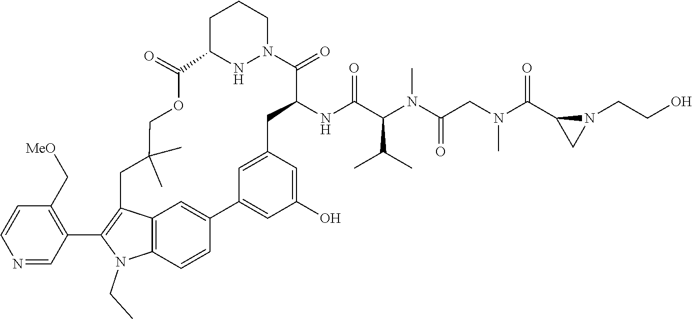 |
| B63 | 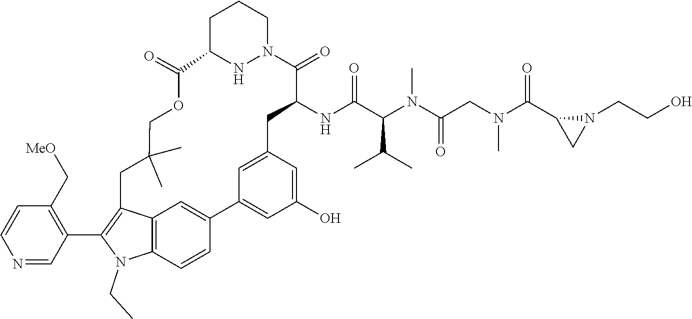 |
| B73 | 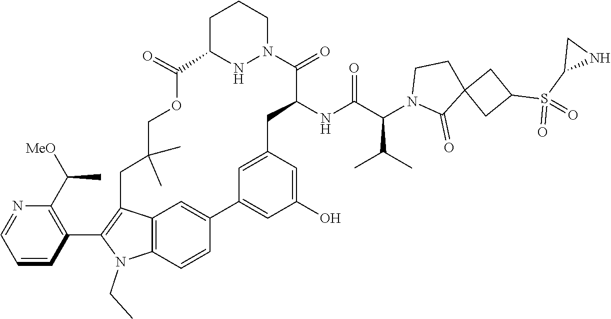 |
| B74 | 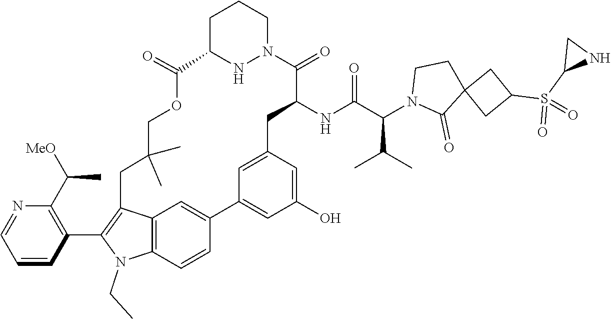 |
| B76 | 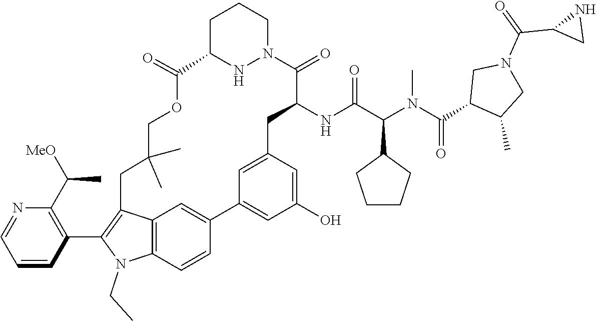 |
| B78 | 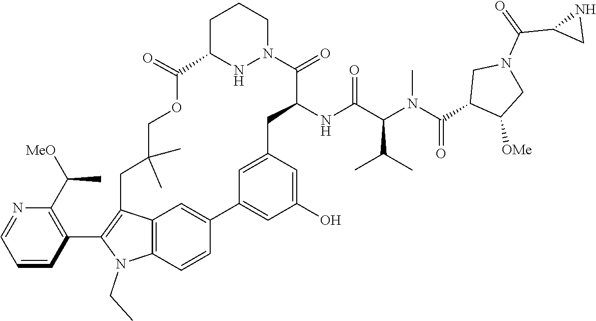 |
| B79 | 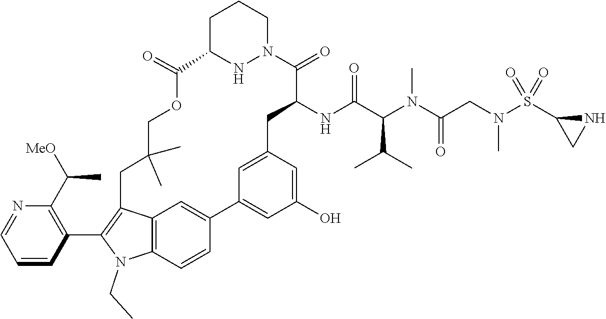 |
| B80 | 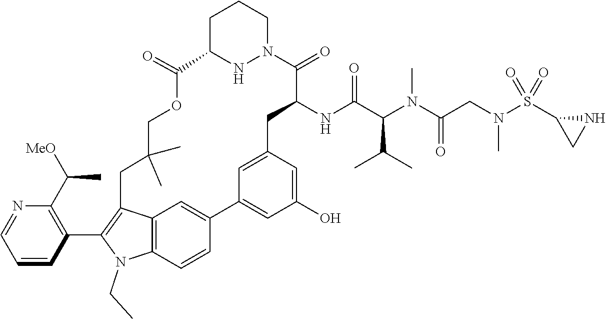 |
| B83 | 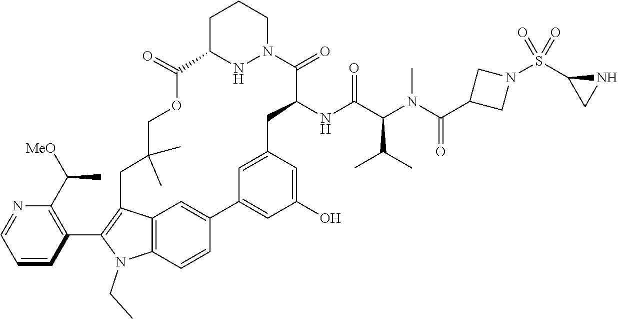 |
| B84 | 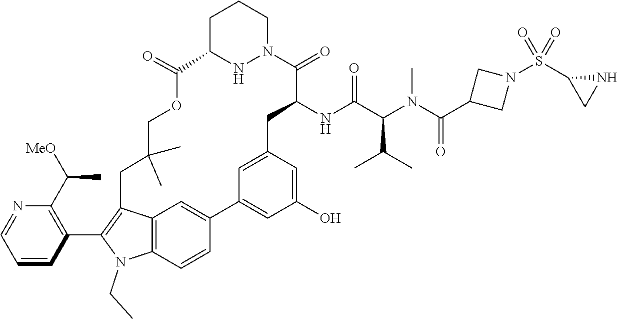 |
| B87 | 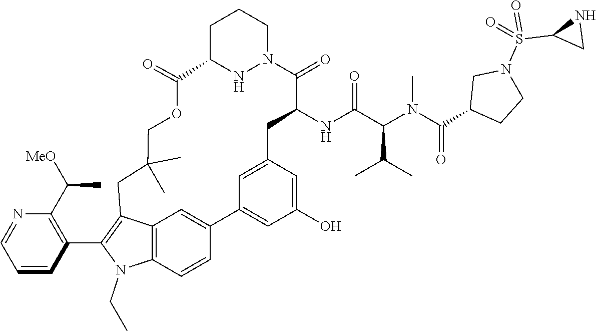 |
| B88 | 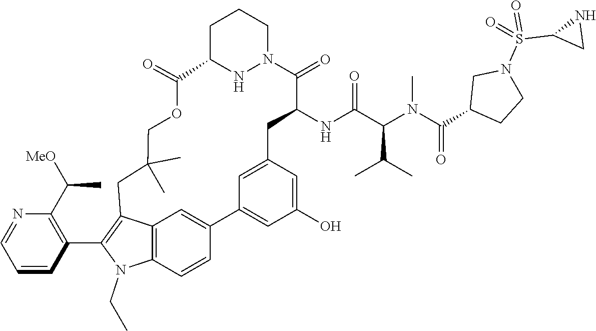 |
| B91 | 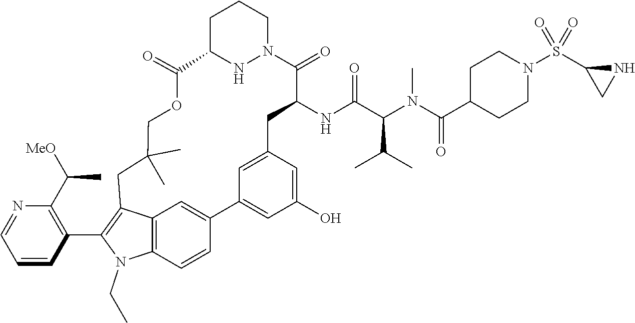 |
| B92 | 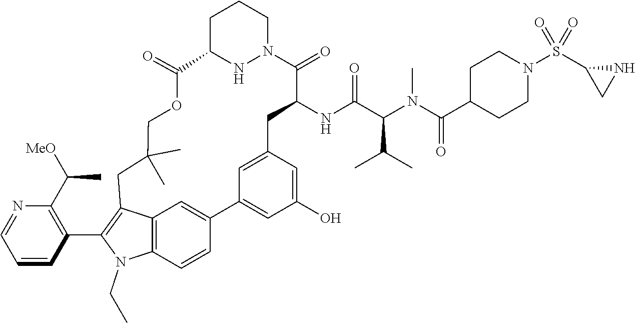 |
| B97 | 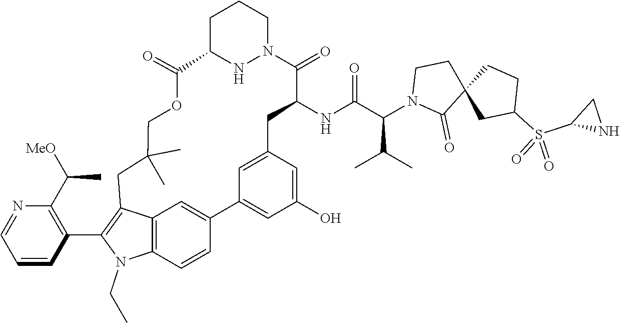 |
| B98 | 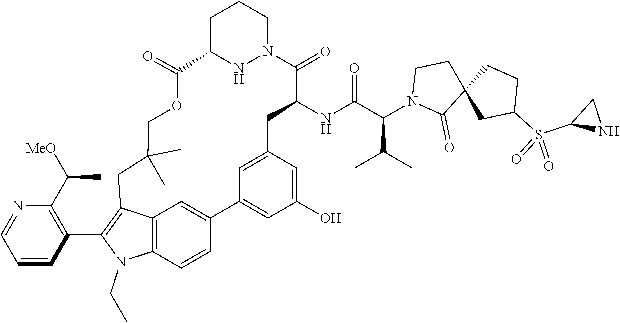 |
| B101 | 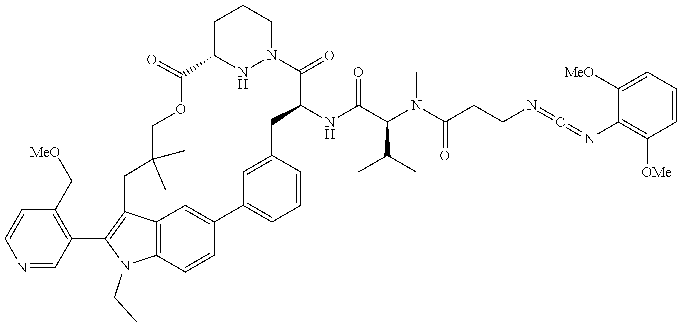 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cccc1OC)C(=O)N1CCC[C@H](N1)C(=O)OCC(C)(C)C2] |
| B108 | 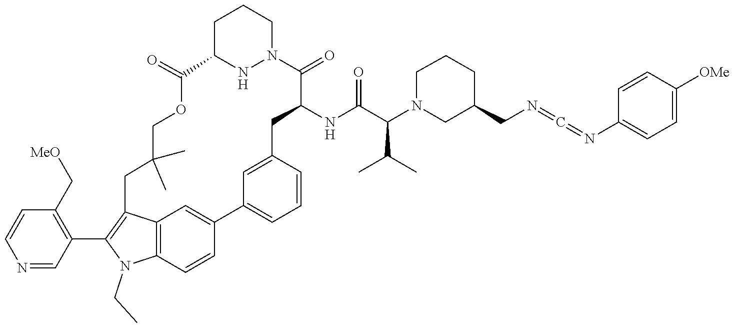 [SMILES: CCn1c(-c2cnccc2COC)c2c3cc(ccc31)-c1cccc(c1)C[C@H](NC(=O)[C@H](C(C)C)N1CCC[C@@H](CN=C=Nc3ccc(OC)cc3)C1)C(=O)N1CCC[C@H](N1)C(=O)OCC(C)(C)C2] |
| B109 | 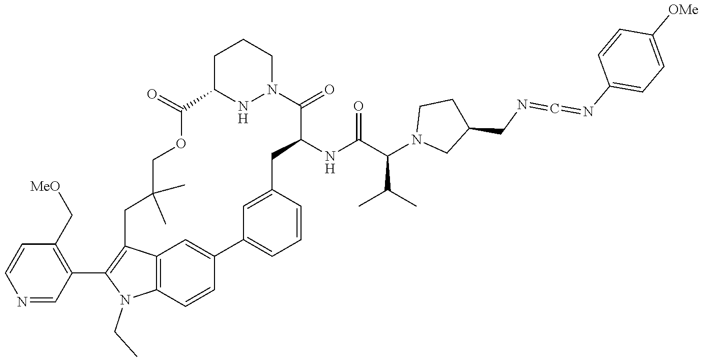 [SMILES: CCn1c(-c2cnccc2COC)c2c3cc(ccc31)-c1cccc(c1)C[C@H](NC(=O)[C@H](C(C)C)N1CC[C@@H](CN=C=Nc3ccc(OC)cc3)C1)C(=O)N1CCC[C@H](N1)C(=O)OCC(C)(C)C2] |
| B113 | 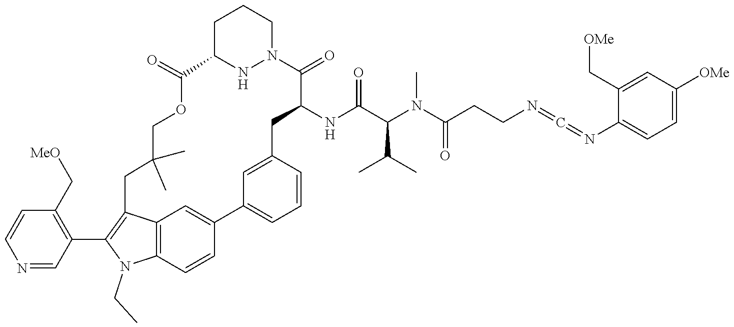 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1COC)C(=O)N1CCC[C@H](N1)C(=O)OCC(C)(C)C2] |
| B116 | 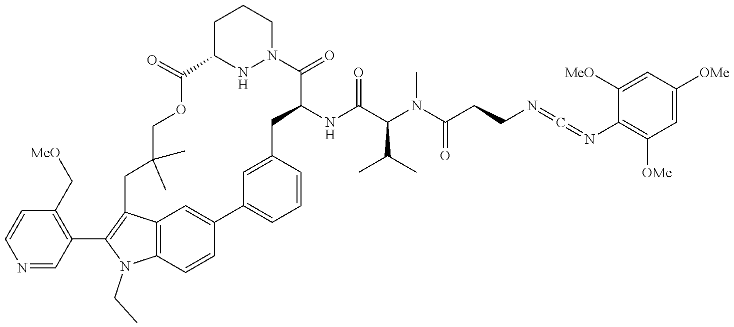 |
| B117 | 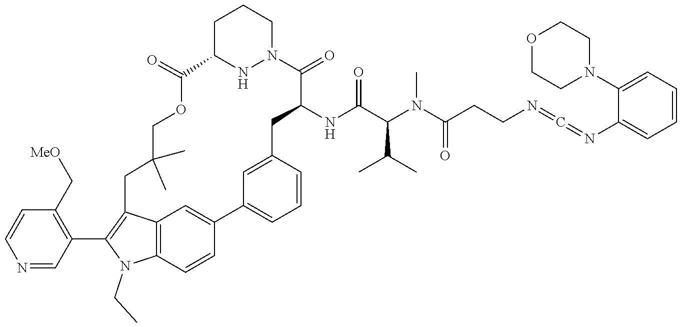 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1N1CCOCC1)C(=O)N1CCC[C@H](N1)C(=O)OCC(C)(C)C2] |
| B118 | 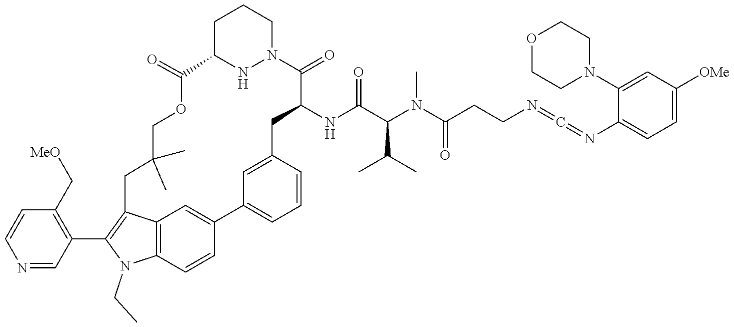 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1N1CCOCC1)C(=O)N1CCC[C@H](N1)C(=O)OCC(C)(C)C2] |
| B119 | 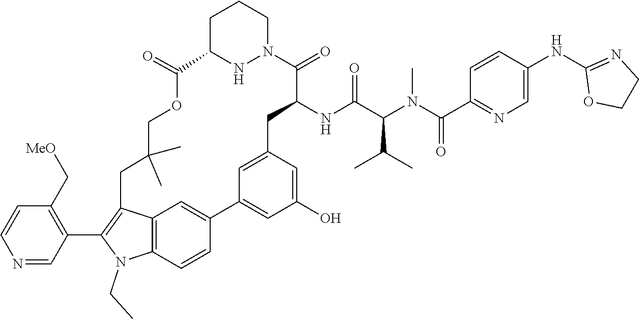 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC3=NCCO3)cn1)C(=O)N1CCC[C@H](N1)C(=O)OCC(C)(C)C2] |
| B120 | 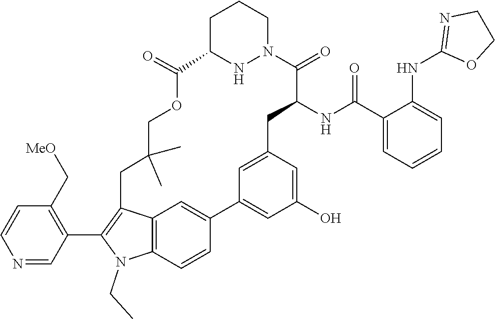 [SMILES: CCn1c(-c2cnccc2COC)c2c3cc(ccc31)-c1cc(O)cc(c1)C[C@H](NC(=O)c1ccccc1NC1=NCCO1)C(=O)N1CCC[C@H](N1)C(=O)OCC(C)(C)C2] |
| B122 | 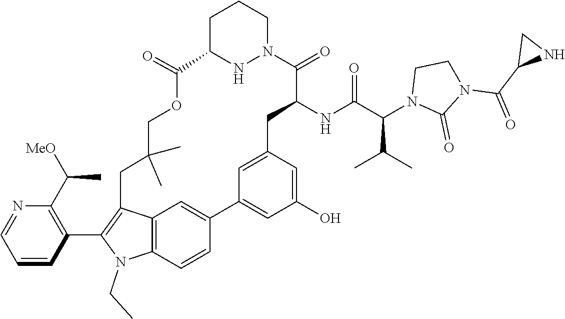 |
| B123 | 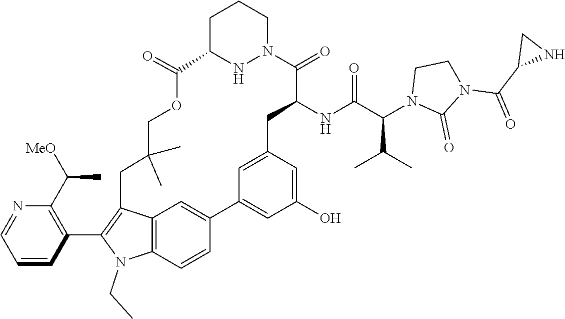 |

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Further provided is a conjugate, or salt thereof, comprising the structure of Formula IV:

M-L-P Formula IV

wherein L is a linker;

P is a monovalent organic moiety; and

M has the structure of Formula Va:

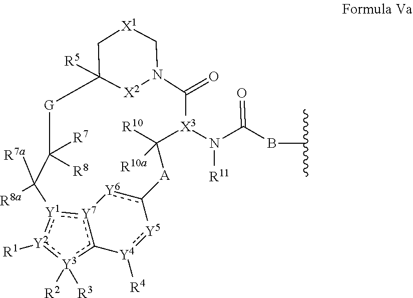

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo; and

R¹¹ is hydrogen or C₁-C₃ alkyl.

In some embodiments, the conjugate has the structure of Formula IV:

M-L-P Formula IV

wherein L is a linker;

P is a monovalent organic moiety; and

M has the structure of Formula Vb:

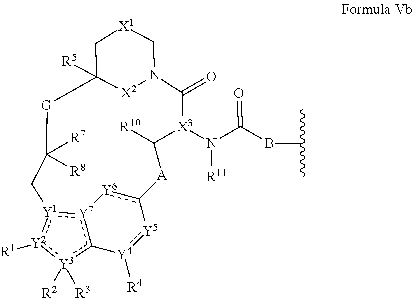

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments, the conjugate has the structure of Formula IV:

M-L-P Formula IV

wherein L is a linker;

P is a monovalent organic moiety; and

M has the structure of Formula Vc:

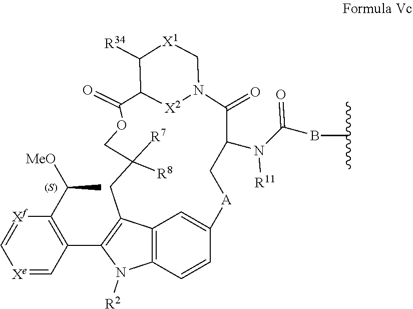

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

X^{e} and X^{f} are, independently, N or CH;

R² is C₁-C₆ alkyl or 3 to 6-membered cycloalkyl;

R⁷ is C₁-C₃ alkyl;

R⁶ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹¹ is hydrogen or C₁-C₃ alkyl; and

R³⁴ is hydrogen or C₁-C₃ alkyl.

In some embodiments of a compound of the present invention, X^{e} is N and X^{f} is CH. In some embodiments, X^{e} is CH and X^{f} is N.

In some embodiments, the conjugate has the structure of Formula IV:

M-L-P Formula IV

wherein L is a linker;

P is a monovalent organic moiety; and

M has the structure of Formula Vd:

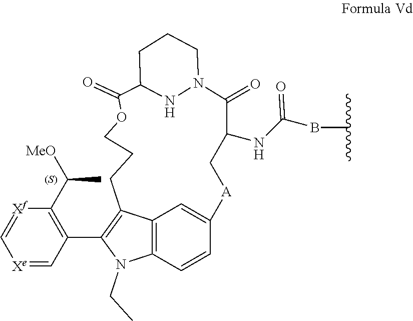

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

X^{e} and X^{f} are, independently, N or CH.

In some embodiments of a compound of the present invention, X^{e} is N and X^{f} is CH. In some embodiments, X^{e} is CH and X^{f} is N.

In some embodiments of conjugates of the present invention, the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A². In some embodiments of conjugates of the present invention, the linker is bound to the monovalent organic moiety through a bond to a carboxyl group of an amino acid residue of the monovalent organic moiety.

In some embodiments of conjugates of the present invention, the monovalent organic moiety is a protein. In some embodiments, the protein is a Ras protein. In some embodiments, the Ras protein is K-Ras G12D or K-Ras G13D.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as K-Ras G12D or K-Ras G13D. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras G12D or K-Ras G13D. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

Compounds of Table 1 herein were prepared using methods disclosed herein or were prepared using methods disclosed herein combined with the knowledge of one of skill in the art. Compounds of Table 2 may be prepared using methods disclosed herein or may be prepared using methods disclosed herein combined with the knowledge of one of skill in the art.

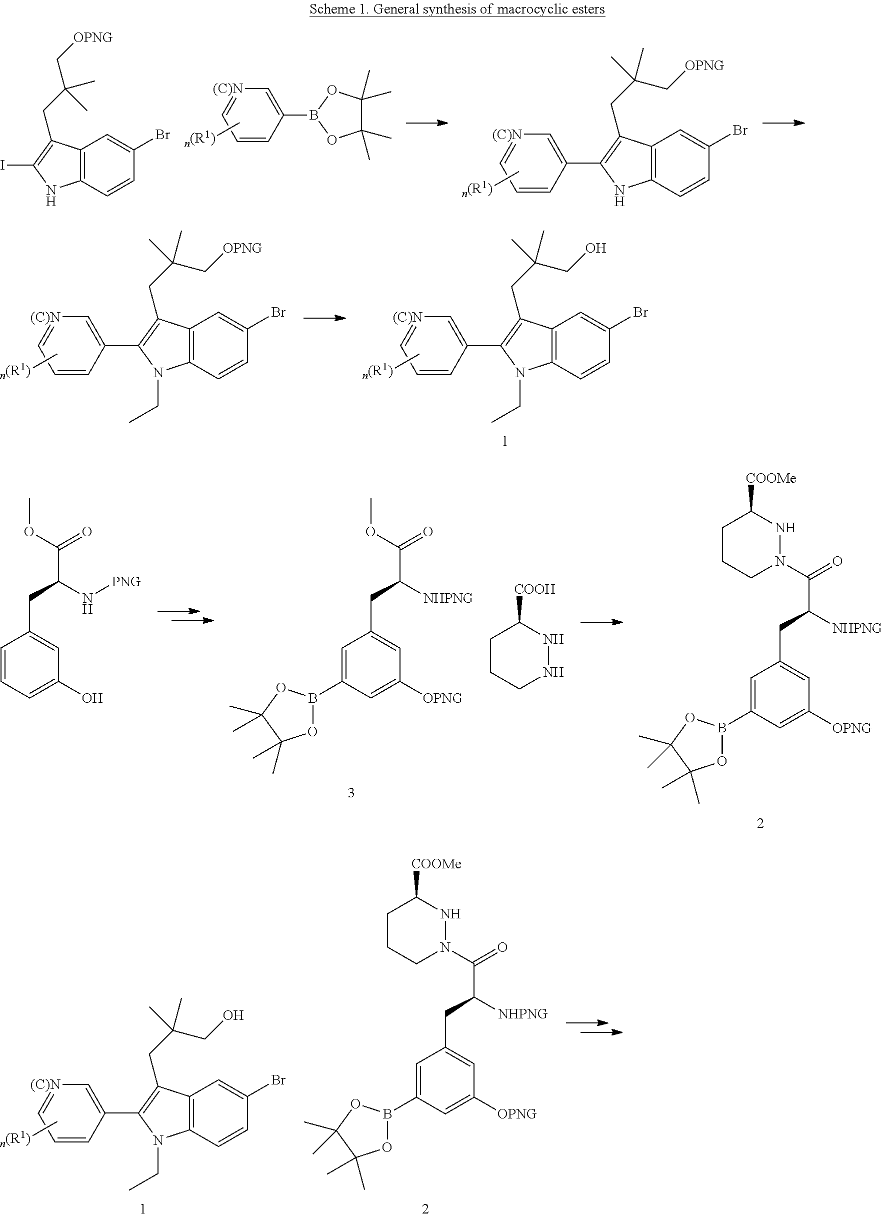

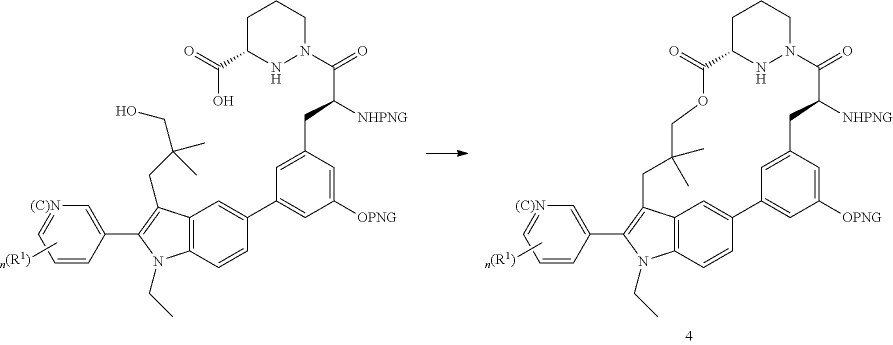

A general synthesis of macrocyclic esters is outlined in Scheme 1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl (S)-hexahydropyridazine-3-carboxylate.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of Pd catalyst followed by hydrolysis and macrolactonization steps to result in an appropriately protected macrocyclic intermediate (4). Additional deprotection or functionalization steps are required to produce a final compound. For example, a person of skill in the art would be able to install into a macrocyclic ester a desired —B-L-W group of a compound of Formula (I), where B, L and W are defined herein, including by using methods exemplified in certain Schemes below and in the Example section herein.

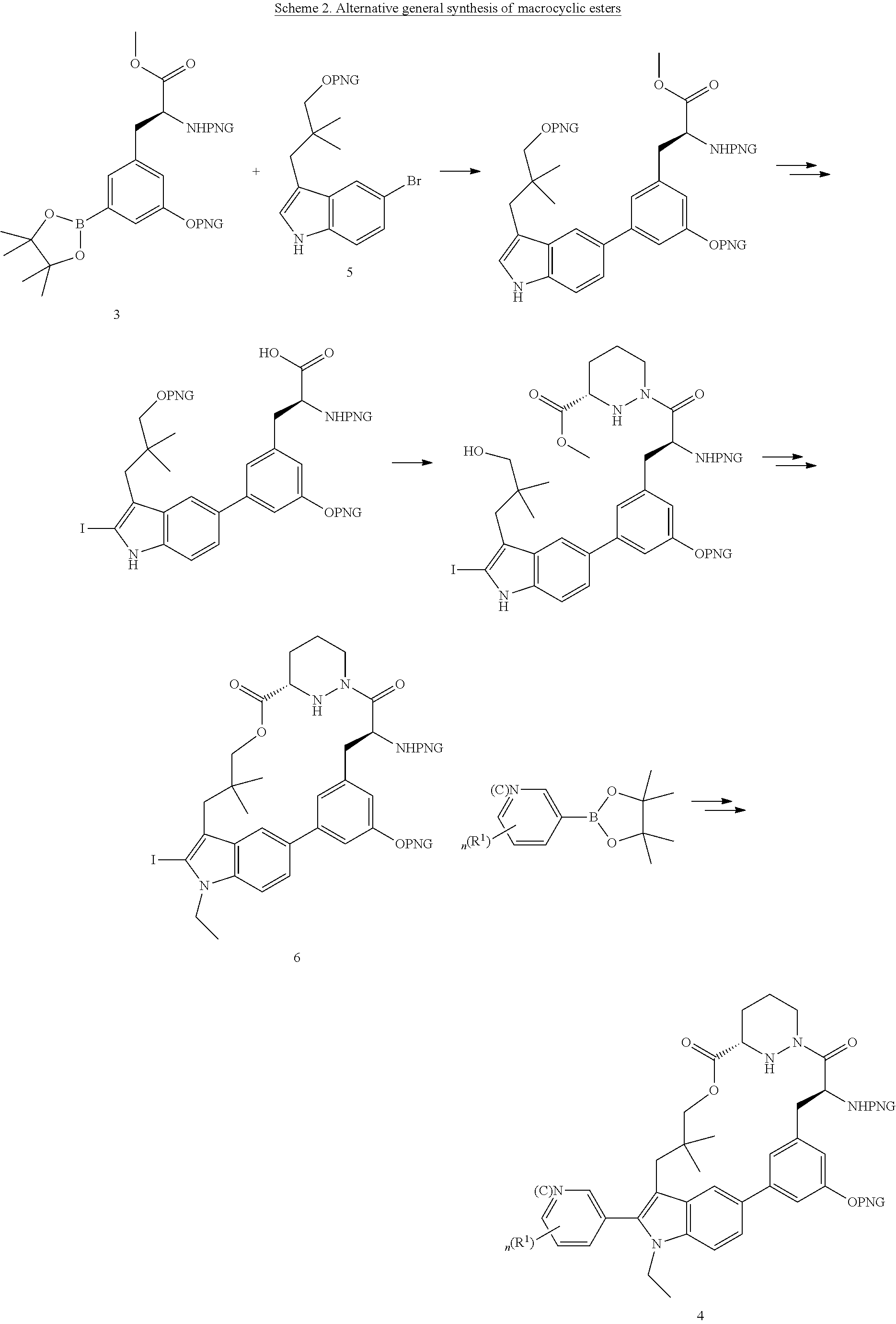

Alternatively, macrocyclic esters can be prepared as described in Scheme 2. An appropriately protected bromo-indolyl (5) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (6). Coupling in the presence of Pd catalyst with an appropriately substituted boronic ester can yield fully a protected macrocycle (4). Additional deprotection or functionalization steps are required to produce a final compound. For example, a person of skill in the art would be able to install into a macrocyclic ester a desired —B-L-W group of a compound of Formula (I), where B, L and W are defined herein, including by using methods exemplified in certain Schemes below and in the Example section herein.

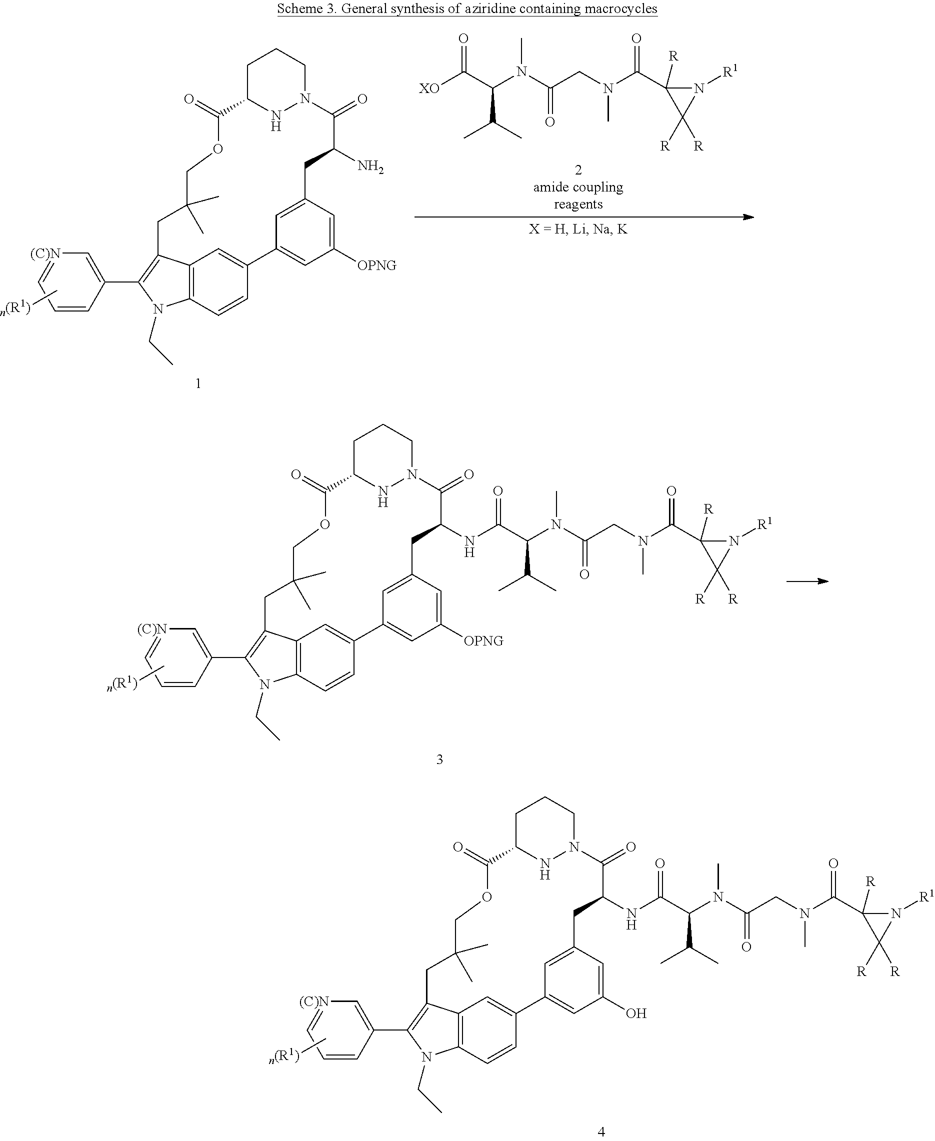

As shown in Scheme 3, compounds of this type may be prepared by the reaction of an appropriate amine (1) with an aziridine containing carboxylic acid (2) in the presence of standard amide coupling reagents, followed by deprotection of the aziridine, if R¹ is a protecting group, and deprotection of the phenol, if required, to produce the final compound (4).

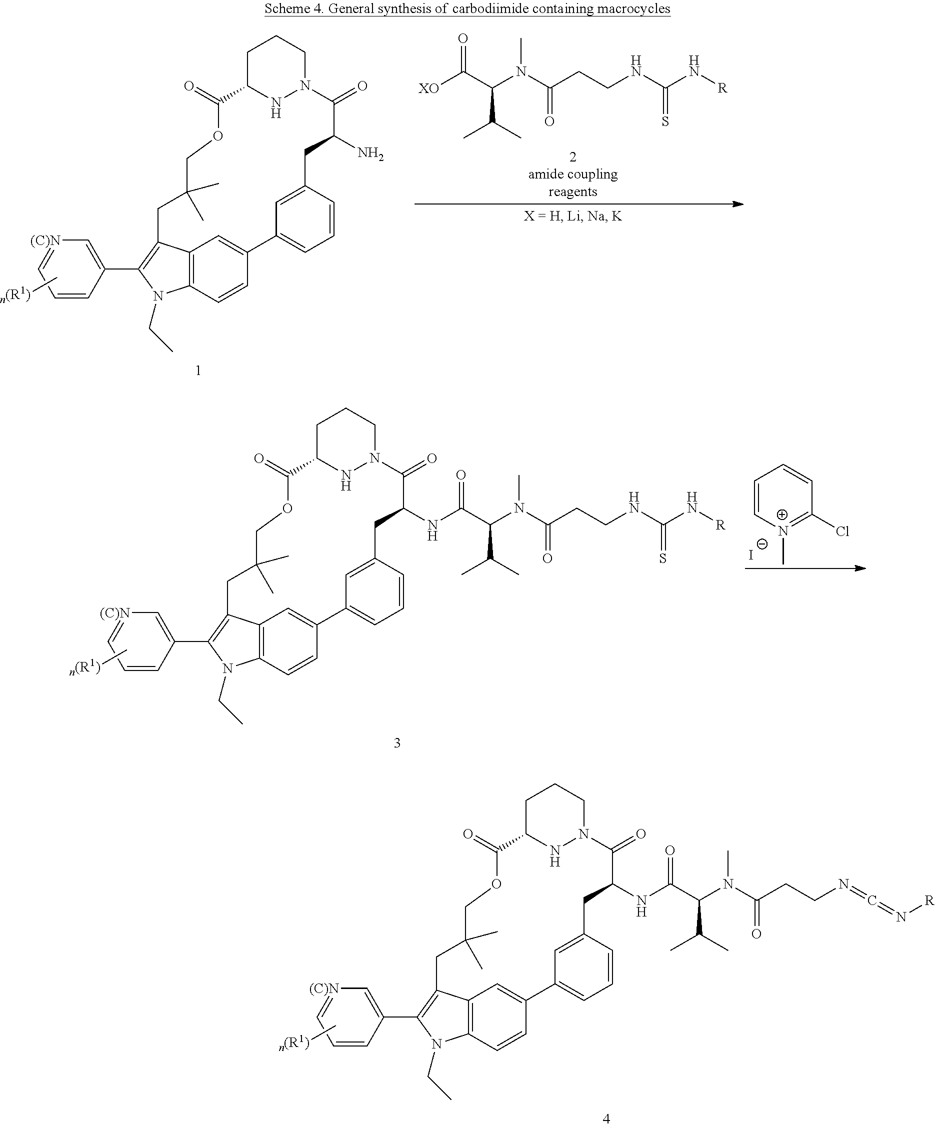

As shown in Scheme 4, compounds of this type may be prepared by the reaction of an appropriate amine (1) with a thiourea containing carboxylic acid (2) in the presence of standard amide coupling reagents, followed by conversion of the thiourea (3) to a carbodiimide (4) in the presence of 2-chloro-1-methylpyridin-1-ium iodide.

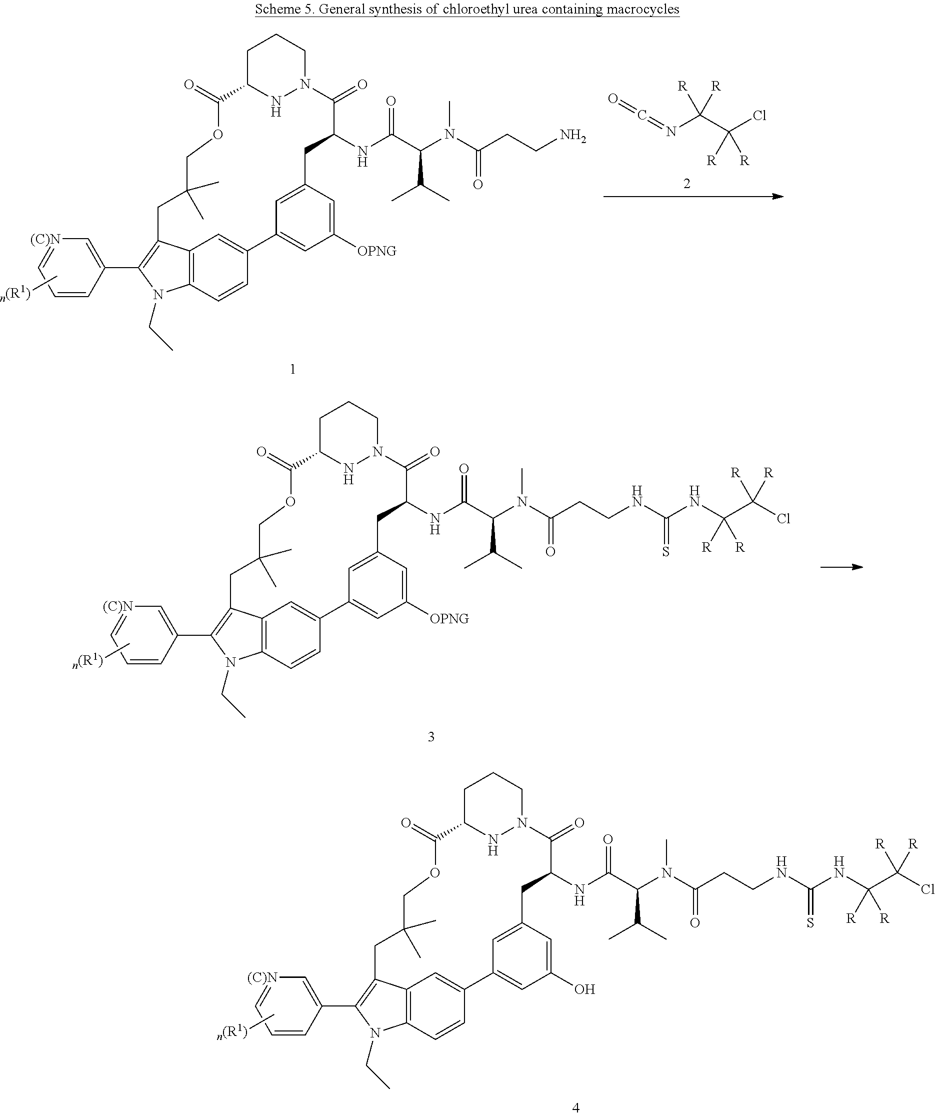

As shown in Scheme 5, compounds of this type may be prepared by the reaction of an appropriate amine (1) with an isocyanate (2) under basic conditions, followed by deprotection of the phenol, if required, to produce the final compound (4).

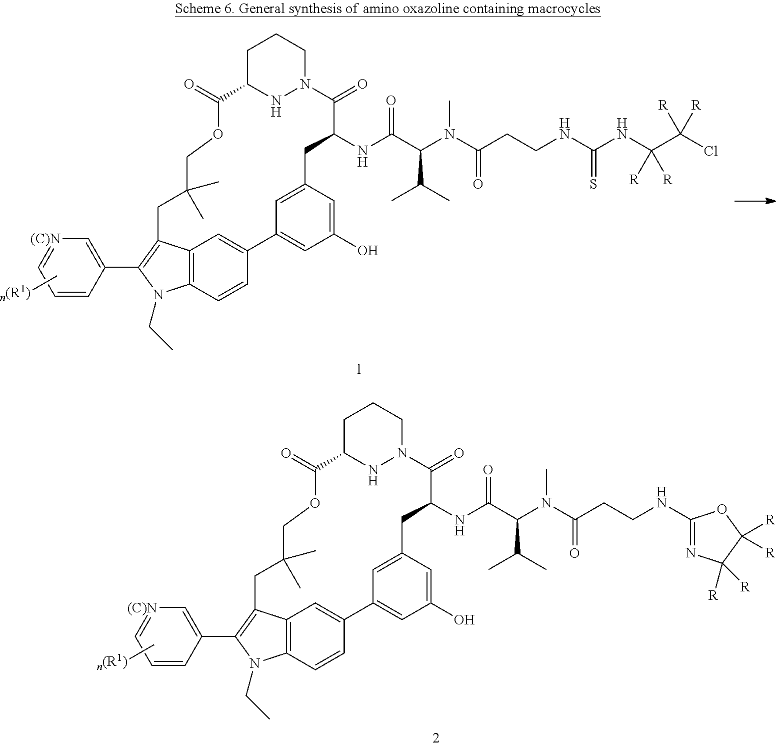

As shown in Scheme 6, compounds of this type may be prepared by cyclization of an appropriate chloroethyl urea (1) under elevated temperatures to produce the final compound (2).

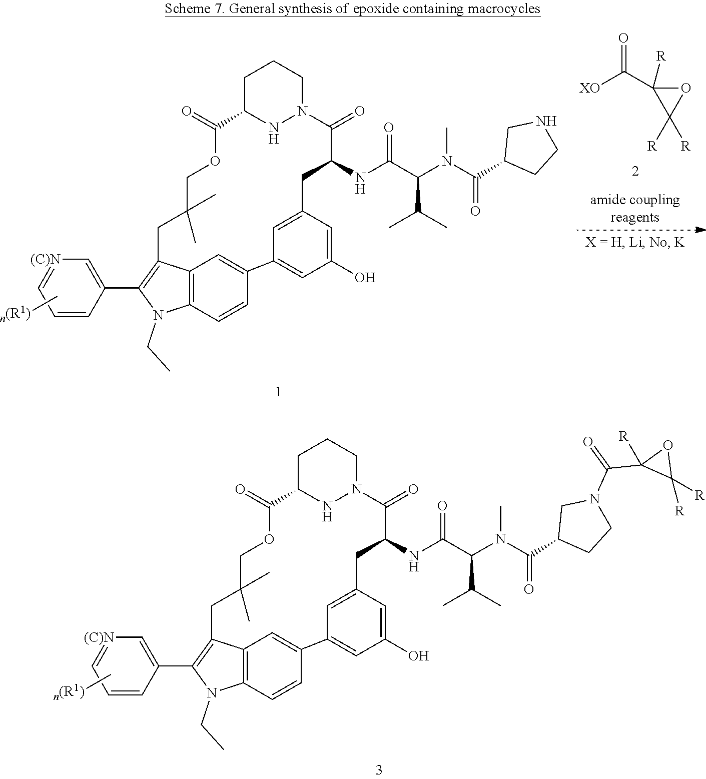

As shown in Scheme 7, compounds of this type may be prepared by the reaction of an appropriate amine (1) with an epoxide containing carboxylic acid (2) in the presence of standard amide coupling reagents to produce the final compound (3).

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (I), where B, L and W are defined herein, including by using methods exemplified in certain Schemes above and in the Example section herein.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition, or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol, and the like. Such compositions may also contain amounts of nontoxic auxiliary substances such as wetting or emulsifying agents, pH buffering agents, and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery, and intranasal administration. Oral administration is also suitable for compounds of the invention, or pharmaceutically acceptable salts thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Numbered Embodiments

[1] A compound, or pharmaceutically acceptable salt thereof, having the structure of Formula I:

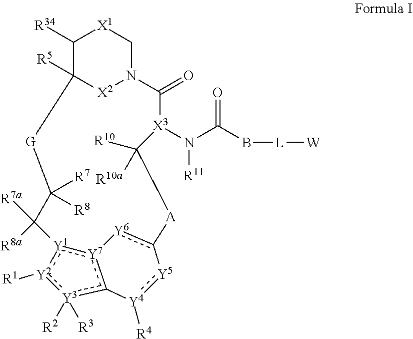

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂ or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R³⁴ is hydrogen or C₁-C₃ alkyl.

[2] The compound, or pharmaceutically acceptable salt thereof, of paragraph [1], wherein G is optionally substituted C₁-C₄ heteroalkylene.

[3] The compound, or pharmaceutically acceptable salt thereof, of paragraph [1] or [2], wherein the compound has the structure of Formula Ia:

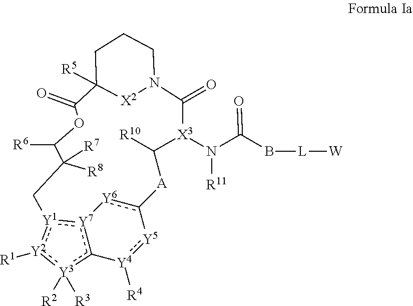

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

[4] The compound, or pharmaceutically acceptable salt thereof, of any one of paragraphs [1] to [3], wherein X² is NH.

[5] The compound, or pharmaceutically acceptable salt thereof, of any one of paragraphs [1] to [4], wherein X³ is CH.

[6] The compound, or pharmaceutically acceptable salt thereof, of any one of paragraphs [1] to [5], wherein R¹¹ is hydrogen.

[7] The compound, or pharmaceutically acceptable salt thereof, of any one of paragraphs [1] to [5], wherein R¹¹ is C₁-C₃ alkyl.

[8] The compound, or pharmaceutically acceptable salt thereof, of paragraph [7], wherein R¹¹ is methyl.

[9] The compound, or pharmaceutically acceptable salt thereof, of any one of paragraphs [1] to [6], wherein the compound has the structure of Formula Ib:

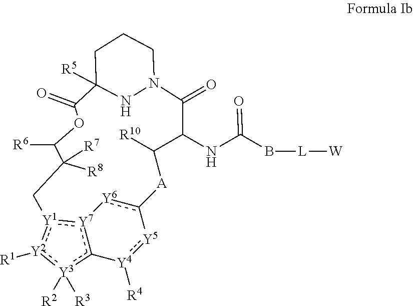

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

[10] The compound, or pharmaceutically acceptable salt thereof, of any one of paragraphs [1] to [9] wherein X¹ is optionally substituted C₁-C₂ alkylene.

[11] The compound, or pharmaceutically acceptable salt thereof, of paragraph [10], wherein X¹ is methylene.

[12] The compound, or pharmaceutically acceptable salt thereof, of any one of paragraphs [1] to [11], wherein R⁵ is hydrogen.

[13] The compound, or pharmaceutically acceptable salt thereof, of any one of paragraphs [1] to [11], wherein R⁵ is C₁-C₄ alkyl optionally substituted with halogen.

[14] The compound, or pharmaceutically acceptable salt thereof, of paragraph [13], wherein R⁵ is methyl.

[15] The compound, or pharmaceutically acceptable salt thereof, of any one of paragraphs [1] to [14], wherein Y⁴ is C.

[16] The compound, or pharmaceutically acceptable salt thereof, of any one of paragraphs [1] to [15], wherein R⁴ is hydrogen.

[17] The compound, or pharmaceutically acceptable salt thereof, of any one of paragraphs [1] to [16], wherein Y⁵ is CH.

[18] The compound, or pharmaceutically acceptable salt thereof, of any one of paragraphs [1] to [17], wherein Y⁶ is CH.

[19] The compound, or pharmaceutically acceptable salt thereof, of any one of paragraphs [1] to [18], wherein Y¹ is C.

[20] The compound, or pharmaceutically acceptable salt thereof, of any one of paragraphs [1] to [19], wherein Y² is C.

[21] The compound, or pharmaceutically acceptable salt thereof, of any one of paragraphs [1] to [20], wherein Y³ is N.

[22] The compound, or pharmaceutically acceptable salt thereof, of any one of paragraphs [1] to [21], wherein R³ is absent.

[23] The compound, or pharmaceutically acceptable salt thereof, of any one of paragraphs [1] to [22], wherein Y⁷ is C.

[24] The compound, or pharmaceutically acceptable salt thereof, of any one of paragraphs [1] to [6] or [9] to [23], wherein the compound has the structure of Formula Ic:

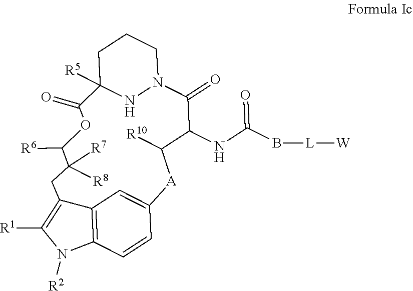

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted Ct-Ce alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

[25] The compound, or pharmaceutically acceptable salt thereof, of any one of paragraphs [1] to [24], wherein R⁶ is hydrogen.

[26] The compound, or pharmaceutically acceptable salt thereof, of any one of paragraphs [1] to [25], wherein R² is hydrogen, cyano, optionally substituted C₁-C₆ alkyl, optionally substituted 3 to 6-membered cycloalkyl, or optionally substituted 3 to 6-membered heterocycloalkyl.

[27] The compound, or pharmaceutically acceptable salt thereof, of paragraph [26], wherein R² is optionally substituted C₁-C₆ alkyl.

[28] The compound, or pharmaceutically acceptable salt thereof, of paragraph [27], wherein R² is ethyl.

[29] The compound, or pharmaceutically acceptable salt thereof, of any one of paragraphs [1] to [28], wherein R⁷ is optionally substituted C₁-C₃ alkyl.

[30] The compound, or pharmaceutically acceptable salt thereof, of paragraph [29], wherein R⁷ is C₁-C₃ alkyl.

[31] The compound, or pharmaceutically acceptable salt thereof, of any one of paragraphs [1] to [30], wherein R⁸ is optionally substituted C₁-C₃ alkyl.

[32] The compound, or pharmaceutically acceptable salt thereof, of paragraph [31], wherein R⁸ is C₁-C₃ alkyl.

[33] The compound, or pharmaceutically acceptable salt thereof, of any one of paragraphs [1] to [32], wherein the compound has the structure of Formula Id:

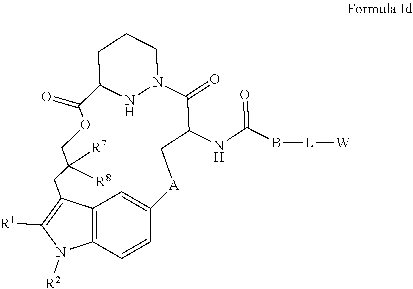

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is C₁-C₆ alkyl or 3 to 6-membered cycloalkyl;

R⁷ is C₁-C₃ alkyl;

R^{a} is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

[34] The compound, or pharmaceutically acceptable salt thereof, of any one of paragraphs [1] to [33], wherein R¹ is 5 to 10-membered heteroaryl.

[35] The compound, or pharmaceutically acceptable salt thereof, of paragraph [34], wherein R¹ is optionally substituted 6-membered aryl or optionally substituted 6-membered heteroaryl.

[36] The compound, or pharmaceutically acceptable salt thereof, of any one of paragraphs [1] to [35], wherein the compound has the structure of Formula Ie:

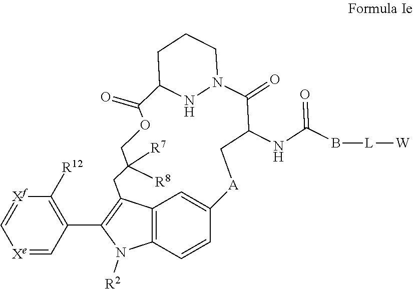

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl

X^{e} and X^{f} are, independently, N or CH; and

R¹² is optionally substituted C₁-C₆ alkyl or optionally substituted C₁-C₆ heteroalkyl, or optionally substituted 3 to 7-membered heterocycloalkyl.

[37] The compound, or pharmaceutically acceptable salt thereof, of paragraph [36], wherein X^{e} is N and X^{f} is CH.

[38] The compound, or pharmaceutically acceptable salt thereof, of paragraph [36], wherein X^{e} is CH and X^{f} is N.

[39] The compound, or pharmaceutically acceptable salt thereof, of any one of paragraphs [36] to [38], wherein R¹² is optionally substituted C₁-C₆ heteroalkyl.

[40] The compound, or pharmaceutically acceptable salt thereof, of any one of paragraphs [36]CH₃ to [39], wherein R¹² is

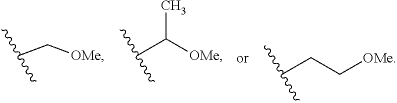

[41] The compound, or pharmaceutically acceptable salt thereof, of paragraph [1] or [2], wherein the compound has the structure of Formula VI:

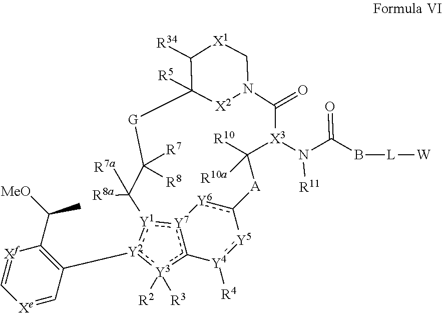

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl;

R³⁴ is hydrogen or C₁-C₃ alkyl; and

X^{e} and X^{f} are, independently, N or CH.

[42] The compound, or pharmaceutically acceptable salt thereof, of paragraph [41], wherein the compound has the structure of Formula VIa:

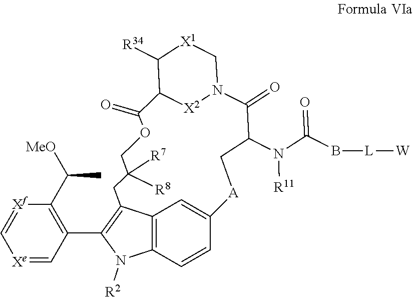

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} and X^{f} are, independently, N or CH;

R¹¹ is hydrogen or C₁-C₃ alkyl; and

R²¹ is hydrogen or C₁-C₃ alkyl.

[43] The compound, or pharmaceutically acceptable salt thereof, of paragraph [41] or [42], wherein the compound has the structure of Formula VIb:

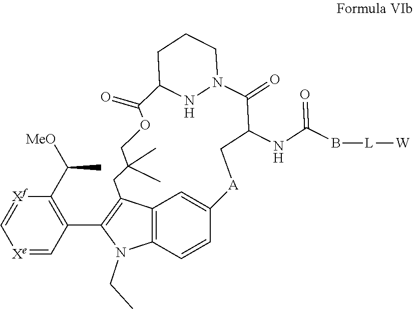

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

X^{e} and X^{f} are, independently, N or CH.

[44] The compound, or pharmaceutically acceptable salt thereof, of any one of paragraphs [1] to [43], wherein A is optionally substituted 6-membered arylene.

[45] The compound, or pharmaceutically acceptable salt thereof, of paragraph [44], wherein A has the structure:

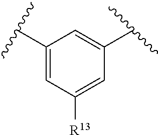

wherein R¹³ is hydrogen, hydroxy, amino, optionally substituted C₁-C₆ alkyl, or optionally substituted C₁-C₆ heteroalkyl.

[46] The compound, or pharmaceutically acceptable salt thereof, of paragraph [45], wherein R¹³ is hydrogen.

[47] The compound, or pharmaceutically acceptable salt thereof, of paragraph [45], wherein R¹³ is hydroxy.

[48] The compound, or pharmaceutically acceptable salt thereof, of any one of paragraphs [1] to [47], wherein B is —CHR⁹—.

[49] The compound, or pharmaceutically acceptable salt thereof, of paragraph [48], wherein R⁹ is optionally substituted C₁-C₆ alkyl or optionally substituted 3 to 6-membered cycloalkyl.

[50] The compound, or pharmaceutically acceptable salt thereof, of paragraph [49], wherein R⁹ is:

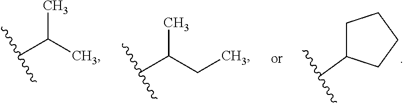

[51] The compound, or pharmaceutically acceptable salt thereof, of paragraph [50], wherein R⁹ is:

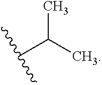 [SMILES: *C(C)C]

[52] The compound, or pharmaceutically acceptable salt thereof, of any one of paragraphs [1] to [47], wherein B is optionally substituted 6-membered arylene.

[53] The compound, or pharmaceutically acceptable salt thereof, of paragraph [52], wherein B is 6-membered arylene.

[54] The compound, or pharmaceutically acceptable salt thereof, of paragraph [53], wherein B is:

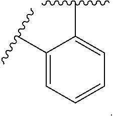 [SMILES: *c1ccccc1*]

[55] The compound, or pharmaceutically acceptable salt thereof, of any one of paragraphs [1] to [54], wherein R⁷ is methyl.

[56] The compound, or pharmaceutically acceptable salt thereof, of any one of paragraphs [1] to [55], wherein R⁸ is methyl.

[57] The compound, or pharmaceutically acceptable salt thereof, of any one of paragraphs [1] to [56], wherein the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A².

[58] The compound, or a pharmaceutically acceptable salt thereof, of any one of paragraphs [1] to [57], wherein the linker is acyclic.

[59] The compound, or a pharmaceutically acceptable salt thereof, of paragraph [58], wherein the linker has the structure of Formula IIa:

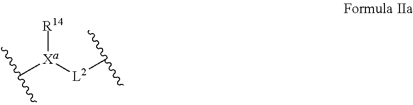

wherein X^{a} is absent or N;

R¹⁴ is absent, hydrogen or optionally substituted C₁-C₆ alkyl; and

L² is absent, —SO₂—, optionally substituted C₁-C₄ alkylene or optionally substituted C₁-C₄ heteroalkylene,

wherein at least one of X^{a}, R¹⁴, or L² is present.

[60] The compound, or a pharmaceutically acceptable salt thereof, of paragraph [59], wherein the linker has the structure:

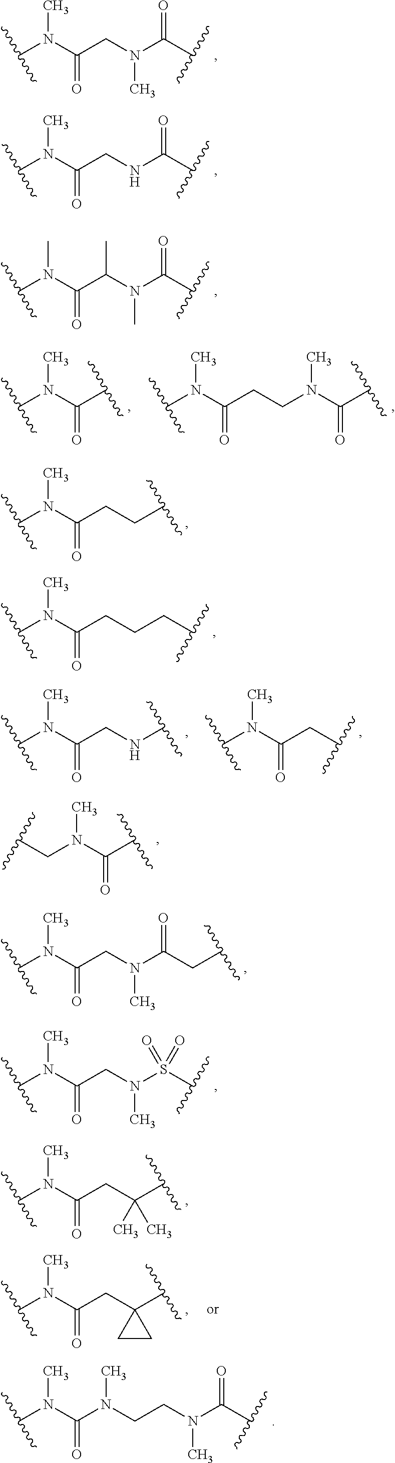

[61] The compound, or a pharmaceutically acceptable salt thereof, of any one of paragraphs [1] to [57], wherein the linker is or a comprises a cyclic group.

[62] The compound, or a pharmaceutically acceptable salt thereof, of any one of paragraphs [1] to [57] or [61], wherein the linker has the structure of Formula lib:

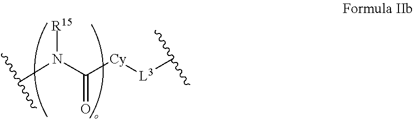

wherein o is 0 or 1;

R¹⁵ is hydrogen or optionally substituted C₁-C₆ alkyl; Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —SO₂—, optionally substituted C₁-C₄ alkylene or optionally substituted C₁-C₄ heteroalkylene.

[63] The compound, or a pharmaceutically acceptable salt thereof, of paragraph [62], wherein the linker has the structure:

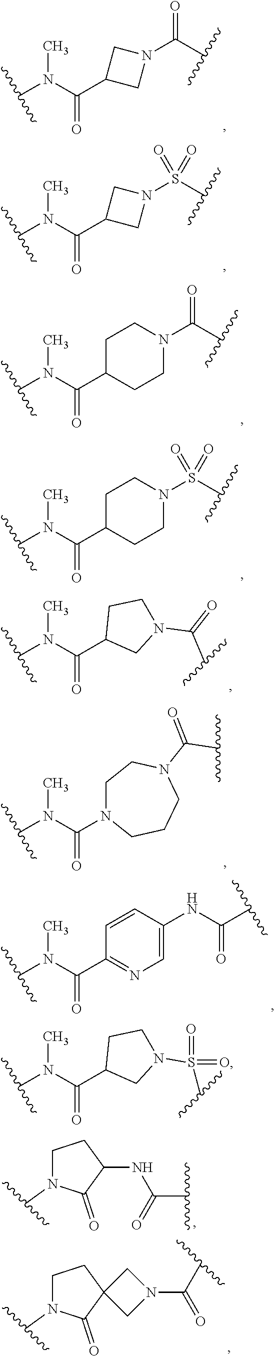

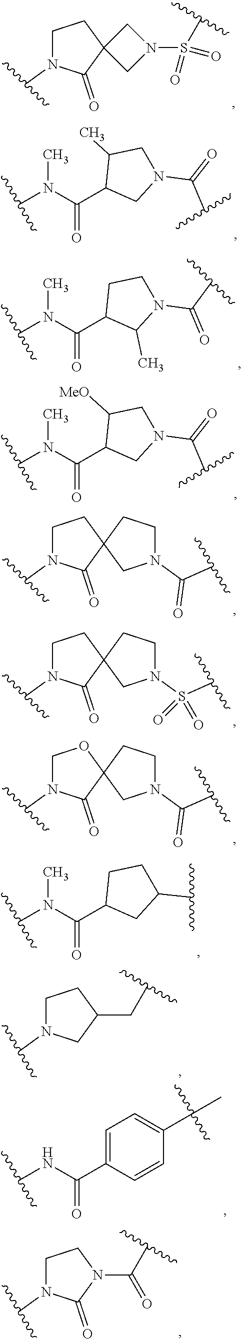

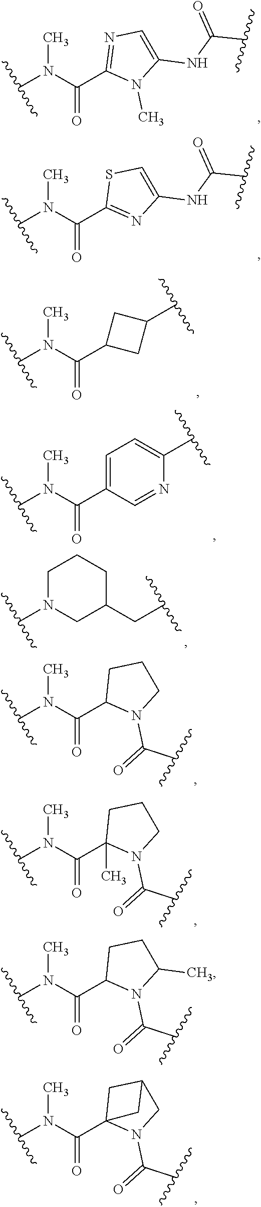

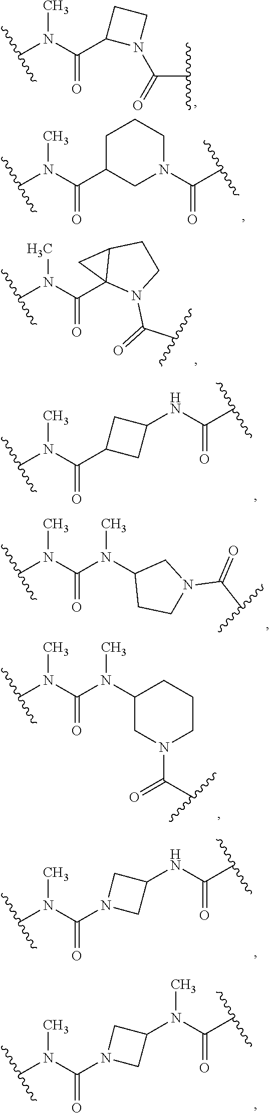

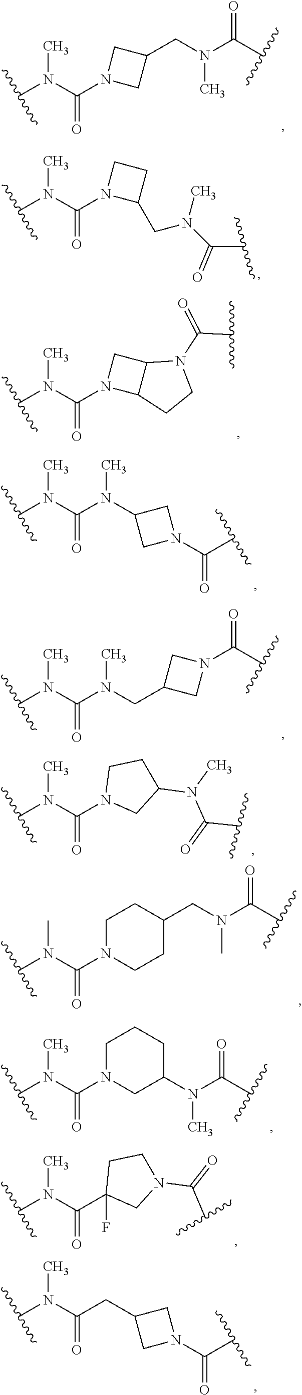

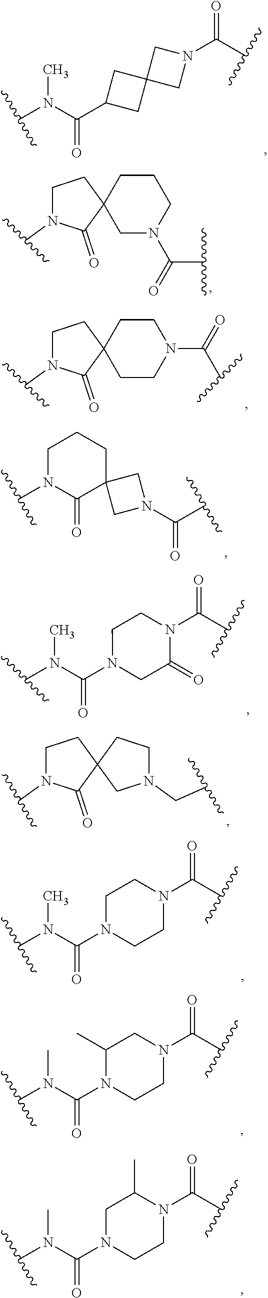

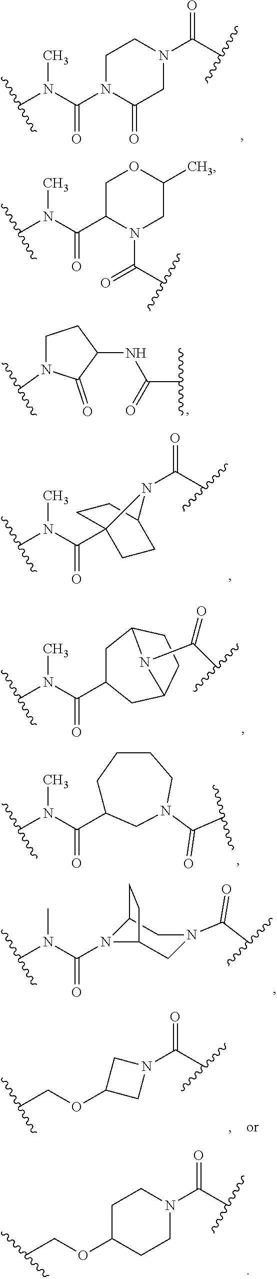

[64] The compound, or a pharmaceutically acceptable salt thereof, of any one of paragraphs [1] to [63], wherein W comprises a carbodiimide.

[65] The compound, or a pharmaceutically acceptable salt thereof, of paragraph [64], wherein W has the structure of Formula IIIa:

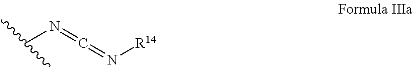

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl.

[66] The compound, or a pharmaceutically acceptable salt thereof, of paragraph [65], wherein W has the structure:

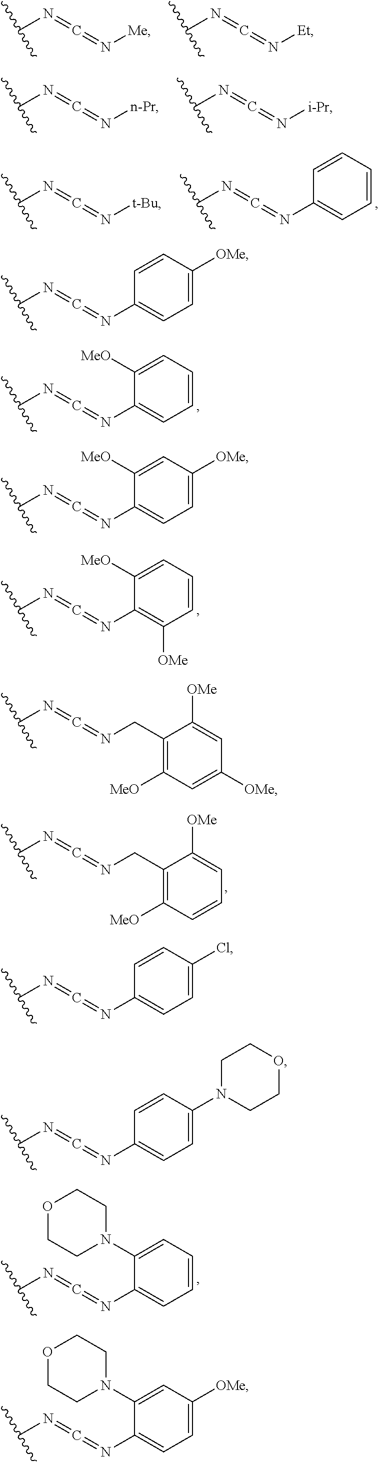

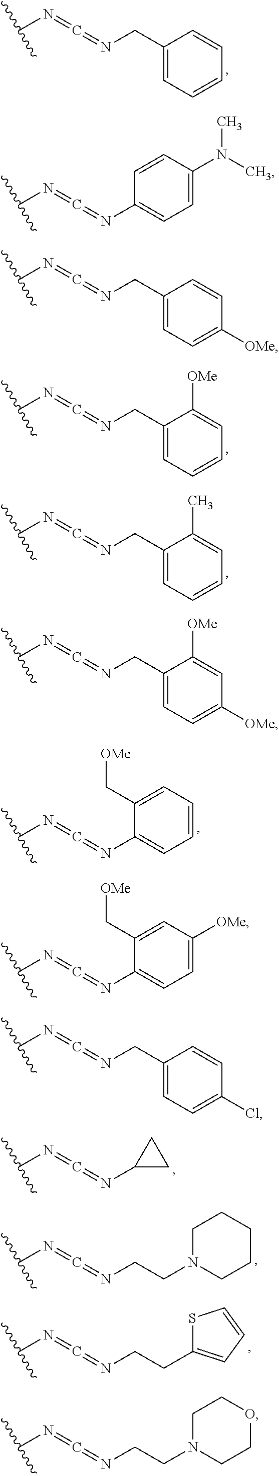

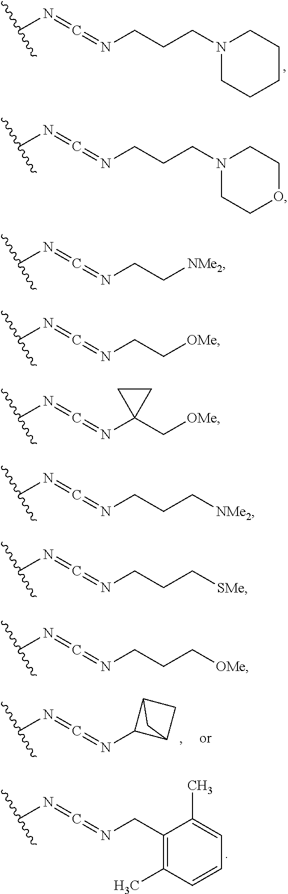

[67] The compound, or a pharmaceutically acceptable salt thereof, of any one of paragraphs [1] to [63], wherein W comprises an oxazoline or thiazoline.

[68] The compound, or a pharmaceutically acceptable salt thereof, of paragraph [67], wherein W has the structure of Formula IIIb:

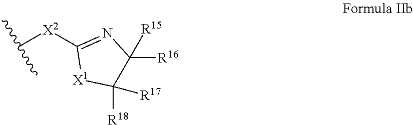

wherein X¹ is O or S;

X² is absent or NR⁹;

R¹⁵, R¹⁶, R¹⁷, and R¹⁸ are, independently, hydrogen or optionally substituted C₁-C₆ alkyl; and

R¹⁹ is hydrogen, C(O)(optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl.

[69] The compound, or a pharmaceutically acceptable salt thereof, of paragraph [68], wherein W is

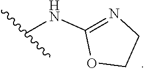 [SMILES: *NC1=NCCO1]

[70] The compound, or a pharmaceutically acceptable salt thereof, of any one of paragraphs [1] to [63], wherein W comprises a chloroethyl urea, a chloroethyl thiourea, a chloroethyl carbamate, or a chloroethyl thiocarbamate.

[71] The compound, or a pharmaceutically acceptable salt thereof, of paragraph [70], wherein W has the structure of Formula IIIc:

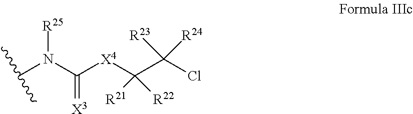

wherein X³ is O or S;

X⁴ is O, S, NR²⁶;

R²¹, R²², R²³, R²⁴, and R²⁶ are, independently, hydrogen or optionally substituted C₁-C₆ alkyl; and

R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl.

[72] The compound, or a pharmaceutically acceptable salt thereof, of paragraph [71], wherein W is

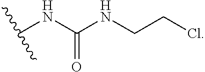 [SMILES: *NC(=O)NCCCl]

[73] The compound, or a pharmaceutically acceptable salt thereof, of any one of paragraphs [1] to [63], wherein W comprises an aziridine.

[74] The compound, or a pharmaceutically acceptable salt thereof, of paragraph [73], wherein W has the structure of Formula IIId1, Formula IIId2, Formula IIId3, or Formula IIId4:

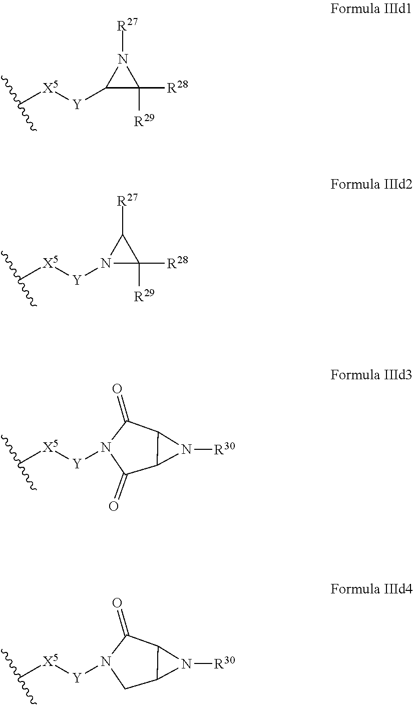

wherein X⁵ is absent or NR³⁰;

Y is absent or C(O), C(S), S(O), SO₂, or optionally substituted C₁-C₃ alkylene;

R²⁷ is hydrogen, —C(O)R³², —C(O)OR³², —SO₂R³³, —SOR³³, optionally substituted C₁-C₆ alkyl, optionally substituted 6 to 10-membered aryl, optionally substituted 3 to 14-membered heterocycloalkyl, or optionally substituted 5 to 10-membered heteroaryl;

R²⁸ and R²⁹ are, independently, hydrogen, CN, C(O)R³¹, CO₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;

each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;

R³⁰ is hydrogen or optionally substituted C₁-C₆ alkyl; and

R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl.

[75] The compound, or a pharmaceutically acceptable salt thereof, of paragraph [73] or [74], wherein W is:

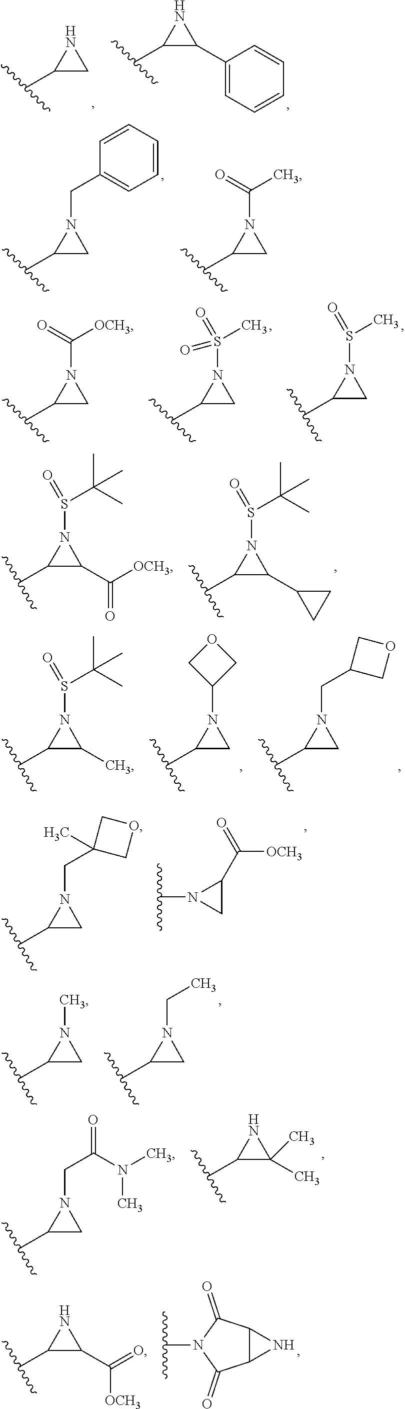

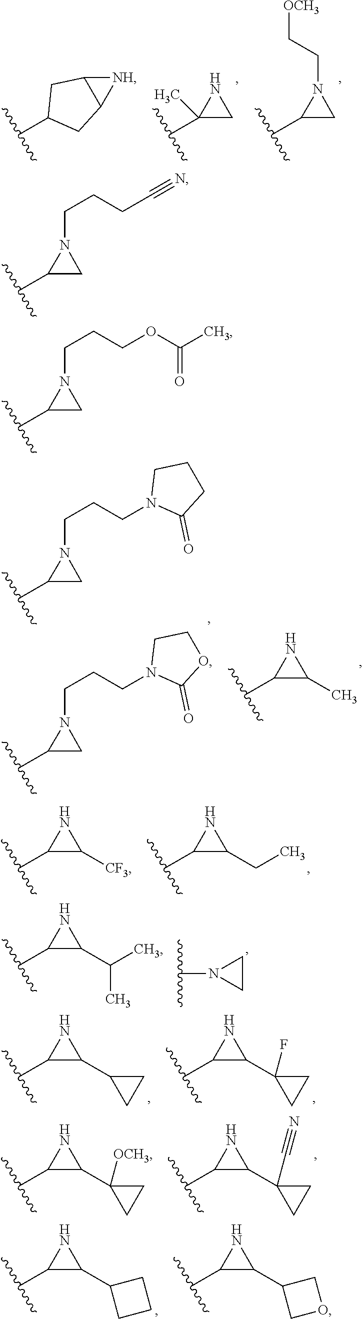

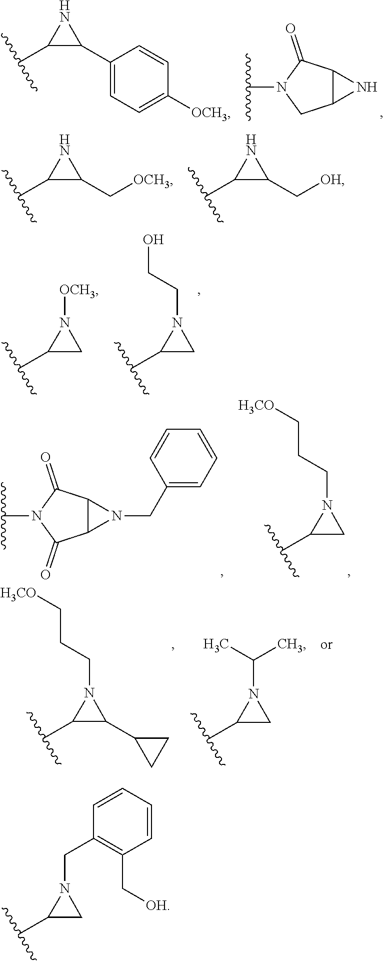

[76] The compound, or a pharmaceutically acceptable salt thereof, of any one of paragraphs [1] to [63], wherein W comprises an epoxide.

[77] The compound, or a pharmaceutically acceptable salt thereof, of paragraph [76], wherein W is

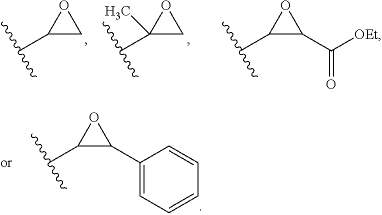

[78] A compound, or a pharmaceutically acceptable salt thereof, of Table 1 or Table 2.

[79] A pharmaceutical composition comprising a compound, or a pharmaceutically acceptable salt thereof, of any one of paragraphs [1] to [78] and a pharmaceutically acceptable excipient.

[80] A conjugate, or salt thereof, comprising the structure of Formula IV:

M-L-P Formula IV

wherein L is a linker;

P is a monovalent organic moiety; and

M has the structure of Formula V:

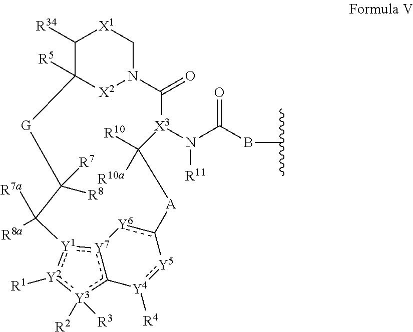

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R^{10a} is hydrogen or halo;

R¹¹ is hydrogen or C₁-C₃ alkyl; and

R³⁴ is hydrogen or C₁-C₃ alkyl.

[81] A conjugate, or salt thereof, of paragraph [80], wherein M has the structure of Formula Vc:

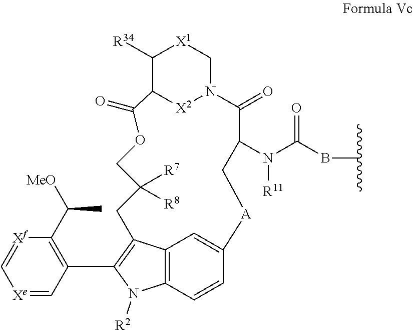

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

X^{e} and X^{f} are, independently, N or CH;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹¹ is hydrogen or C₁-C₃ alkyl; and

R³⁴ is hydrogen or C₁-C₃ alkyl.

In some embodiments of a compound of the present invention, X^{e} is N and X^{f} is CH. In some embodiments, X^{e} is CH and X^{f} is N.

[82] The conjugate, or salt thereof, of paragraph [80] or [81], wherein M has the structure of Formula Vd:

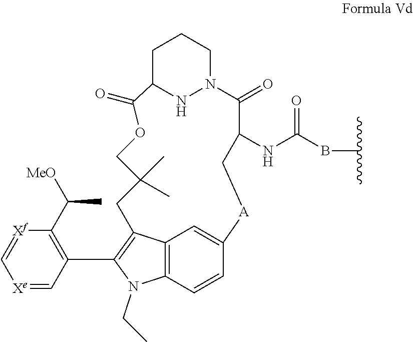

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

X^{e} and X^{f} are, independently, N or CH.

[83] The conjugate, or salt thereof, of any one of paragraphs [80] to [82], wherein the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A².

[84] The conjugate, or salt thereof, of any one of paragraphs [80] to [83], wherein the monovalent organic moiety is a protein.

[85] The conjugate, or salt thereof, of paragraph [84], wherein the protein is a Ras protein.

[86] The conjugate, or salt thereof, of paragraph [85], wherein the Ras protein is K-Ras G12D or K-Ras G13D.

[87] The conjugate, or salt thereof, of any one of paragraphs [80] to [86], wherein the linker is bound to the monovalent organic moiety through a bond to a carboxyl group of an amino acid residue of the monovalent organic moiety.

[88] A method of treating cancer in a subject in need thereof, the method comprising administering to the subject a therapeutically effective amount of a compound, or a pharmaceutically acceptable salt thereof, of any one of paragraphs [1] to [78] or a pharmaceutical composition of paragraph [79].

[89] The method of paragraph [88], wherein the cancer is pancreatic cancer, non-small cell lung cancer, colorectal cancer or endometrial cancer.

[90] The method of paragraph [88] or [89], wherein the cancer comprises a Ras mutation.

[91] The method of paragraph [90], wherein the Ras mutation is K-Ras G12D or K-Ras G13D.

[92] A method of treating a Ras protein-related disorder in a subject in need thereof, the method comprising administering to the subject a therapeutically effective amount of a compound, or a pharmaceutically acceptable salt thereof, of any one of paragraphs [1] to [78] or a pharmaceutical composition of paragraph [79].

[93] A method of inhibiting a Ras protein in a cell, the method comprising contacting the cell with an effective amount of a compound, or a pharmaceutically acceptable salt thereof, of any one of paragraphs [1] to [78] or a pharmaceutical composition of paragraph [79].

[94] The method of paragraph [92] or [93], wherein the Ras protein is K-Ras G12D or K-Ras G13D.

[95] The method of paragraph [93] or [94], wherein the cell is a cancer cell.

[96] The method of paragraph [95], wherein the cancer cell is a pancreatic cancer cell, a non-small cell lung cancer cell, a colorectal cancer cell, or an endometrial cell.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:
CH₂Cl₂, DCM Methylene chloride, Dichloromethane
CH₃CN, MeCN Acetonitrile
CuI Copper (1) iodide
DIPEA Diisopropylethyl amine
DMF N,N-Dimethylformamide
EtOAc Ethyl acetate
h hour
H₂O Water
HCl Hydrochloric acid
K₃PO₄ Potassium phosphate (tribasic)
MeOH Methanol
Na₂SO₄ Sodium sulfate
NMP N-methyl pyrrolidone
Pd(dppf)Cl₂ [1,1′-Bis(diphenylphosphino)ferrocene]dichloropalladium(II)

### Instrumentation

Mass spectrometry data collection took place with a Shimadzu LCMS-2020 or Waters Acquity UPLC with either a ODa detector or SQ Detector 2. Samples were injected in their liquid phase onto a C-18 reverse phase column to remove assay buffer and prepare the samples for the mass spectrometer. The compounds were eluted from the column using an acetonitrile gradient and fed into the mass analyzer. Initial data analysis took place with either Shimadzu LabSolutions or Waters MassLynx. NMR data was collected with either a Bruker AVANCE III HD 400 MHz or a Bruker Ascend 500 MHz instrument and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

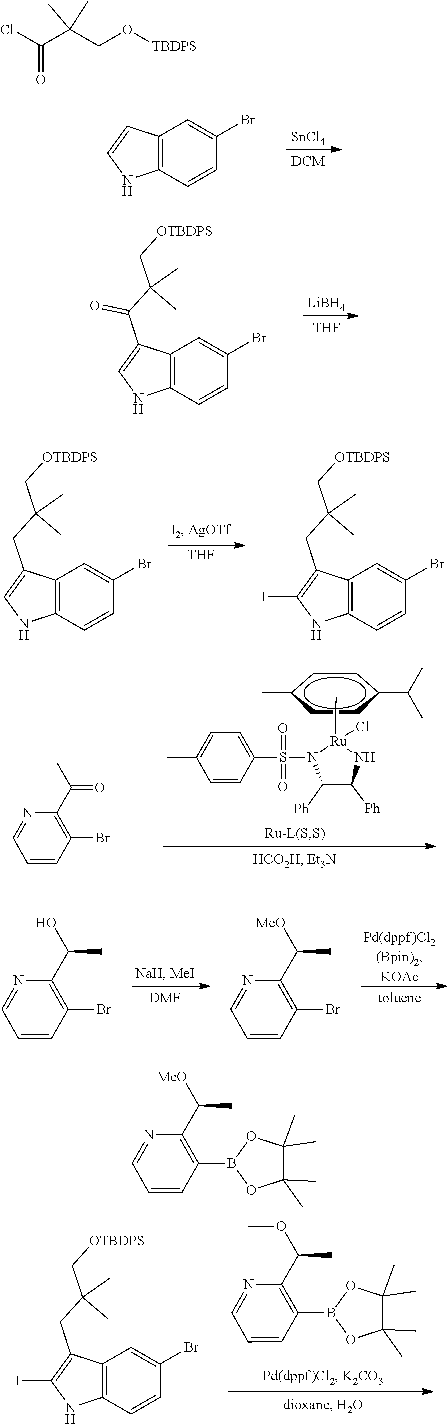

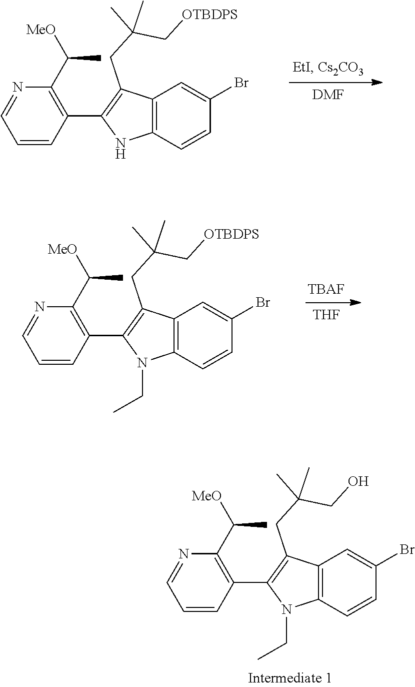

### Step 1: Synthesis of 1-(5-bromo-1H-indol-3-yl)-3-((tert-butyldiphenylsilyl)oxy)-2,2-dimethylpropan-1-one

To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (4×100 mL), dried over Na₂SO₄ and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI) m/z: [M+Na] calcd for C₂₉H₃₂BrNO₂SiNa 556.1; found 556.3.

### Step 2: Synthesis of 1-(5-bromo-1H-indol-3-yl)-3-((tert-butyldiphenylsilyl)oxy)-2,2-dimethylpropan-1-one

To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH·H₂O (890 mg, 4.7 mmol) were added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI) m/z: [M+H] calcd for C₂₉H₃₄BrNOSi: 519.2; found 520.1; ¹H NMR (400 MHz, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

### Step 3: Synthesis of 5-bromo-3-(3-((tert-butyldiphenylsilyl)oxy)-2,2-dimethylpropyl)-2-iodo-1H-indole

To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at room temperature was added AgOTf (888 mg, 3.5 mmol). The mixture was stirred at room temperature for 2 h, then diluted with EtOAc (200 mL) and washed with saturated Na₂S₂O₃ (100 mL), dried over anhydrous Na₂SO₄ and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHz, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

### Step 4: Synthesis of (1S)-1-(3-bromopyridin-2-yl)ethanol

To a stirred mixture of HCOOH (66.3 g, 1.44 mol) in Et₃N (1002 mL, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to room temperature and 1-(3-bromopyridin-2-yl)ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give (1S)-1-(3-bromopyridin-2-yl)ethanol (100 g, 74% yield) a an oil. LCMS (ESI) m/z: [M+H] calcd for C₇H₆BrNO: 201.98; found 201.9.

### Step 5: Synthesis of 3-bromo-2-[(1S)-1-methoxyethyl]pyridine

To a stirred mixture of (1S)-1-(3-bromopyridin-2-yl)ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to room temperature and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI) m/z: [M+H] calcd for C₈H₁₀BrNO: 215.99; found 215.9.

### Step 6: Synthesis of 2-[(1S)-1-methoxyethyl]-3-(4,4,5,5-tetramethyl-1,3,2-dioxaborolan-2-yl)pyridine

To a stirred mixture of 3-bromo-2-[(1S)-1-methoxyethyl]pyridine (90 g, 417 mmol) in toluene (900 mL) at room temperature under an atmosphere of Ar was added bis(pinacolato)diboron (127 g, 500 mmol) and KOAc (81.8 g, 833 mmol) and Pd(dppf)Cl₂ (30.5 g, 41.7 mmol). The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl)pyridine (100 g, 63% yield) as a semi-solid. LCMS (ESI) m/z: [M+H] calcd for C₁₄H₂₂BNO₃: 264.17; found 264.1.

### Step 7: Synthesis of 5-bromo-3-[3-[(tert-butyldiphenylsilyl)oxy]-2,2-dimethylpropyl]-2-[2-[(1S)-1-methoxyethyl]pyridin-3-yl]-1H-indole

To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl)pyridine (100 g, 380 mmol) in 1,4-dioxane (1.4 L) at room temperature under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI) m/z: [M+H] calcd for C₃₇H₄₃SBrN₂O₂Si: 655.23; found 655.1.

### Step 8: Synthesis of 5-bromo-3-[3-[(tert-butyldiphenylsilyl)oxy]-2,2-dimethylpropyl]-1-ethyl-2-[2-[(1S)-1-methoxyethyl]pyridin-3-yl]indole

To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to room temperature and stirred for 16 h then H₂O (4 L) added and the mixture extracted with EtOAc (3×1.5 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI) m/z: [M+H] calcd for C₃₉H₄₇BrN₂O₂Si: 683.26; found 683.3.

### Step 9: Synthesis of 3-(5-bromo-1-ethyl-2-[2-[(1S)-1-methoxyethyl]pyridin-3-yl]indol-3-yl)-2,2-dimethylpropan-1-ol

To a stirred mixture of TBAF (172.6 g, 660 mmol) in THF (660 mL) at room temperature under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI) m/z: [M+H] calcd for C₂₃H₂₉BrN₂O₂: 445.14; found 445.1.

### Intermediate 1. Alternative Synthesis Through Fisher Indole Route

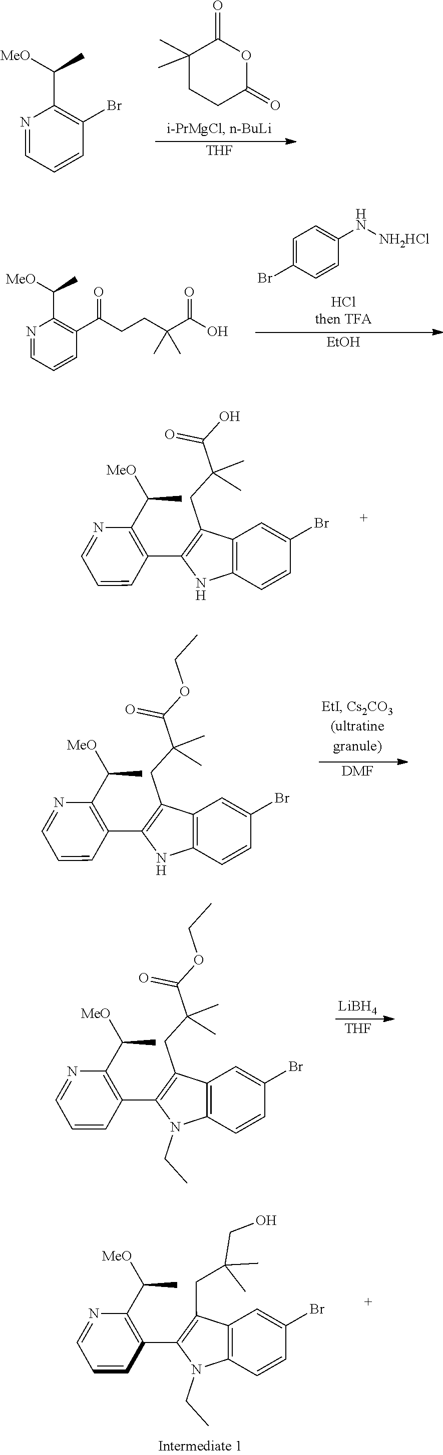

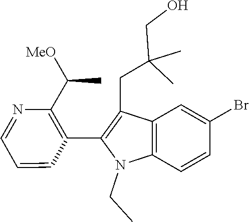

### Step 1: Synthesis of 5-[2-[(1S)-1-methoxyethyl]pyridin-3-yl]-2,2-dimethyl-5-oxopentanoic acid

To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with H₂O (3 L) at 0° C. and extracted with EtOAc (3×2.5 L). The combined organic layers were dried over anhydrous Na₂SO₄, filtered, the filtrate was concentrated under reduced pressure and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI) m/z: [M+H] calcd for C₁₅H₂₁NO₄: 280.15; found 280.1.

### Step 2: Synthesis of 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate

To a mixture of 5-[2-[(1S)-1-methoxyethyl]pyridin-3-yl]-2,2-dimethyl-5-oxopentanoic acid (78 g, 279 mmol) in EtOH (0.78 L) at room temperature under an atmosphere of N₂ was added (4-bromophenyl)hydrazine HCl salt (68.7 g, 307 mmol) in portions. The mixture was heated to 85° C. and stirred for 2 h, cooled to room temperature, then 4M HCl in 1,4-dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5 h, concentrated under reduced pressure and the residue adjusted to pH ˜5 with saturated NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (78 g, crude). LCMS (ESI) m/z: [M+H] calcd for C₂₁H₂₃BrN₂O₃: 430.1 and C₂₃H₂₇BrN₂O₃: 459.12; found 431.1 (carboxylic acid) and 459.1.

### Step 3: Synthesis of ethyl 3-(5-bromo-1-ethyl-2-[2-[(1S)-1-methoxyethyl]pyridin-3-yl]indol-3-yl)-2,2-dimethylpropanoate

To a mixture of 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. EtI (215 g, 1.38 mmol) in DMF (200 mL) was then added dropwise at 0° C. The mixture was warmed to room temperature and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI) m/z: [M+H] calcd for C₂₅H₃₁BrN₂O₃: 487.17; found 487.2.

### Step 4: Synthesis of 3-(5-bromo-1-ethyl-2-(2-((S)-1-methoxyethyl)pyridin-3-yl)-1H-indol-3-yl)-2,2-dimethylpropan-1-ol

To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl)pyridin-3-yl)-1H-indol-3-yl)-2,2-dimethylpropan-1-ol (as single atropisomers) (60 g, 38% yield) and (40 g, 26% yield) both as solids. LCMS (ESI) m/z: [M+H] calcd for C₂₃H₂₉BrN₂O₂: 445.14; found 445.2.

### Intermediate 2 and Intermediate 4. Synthesis of (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate

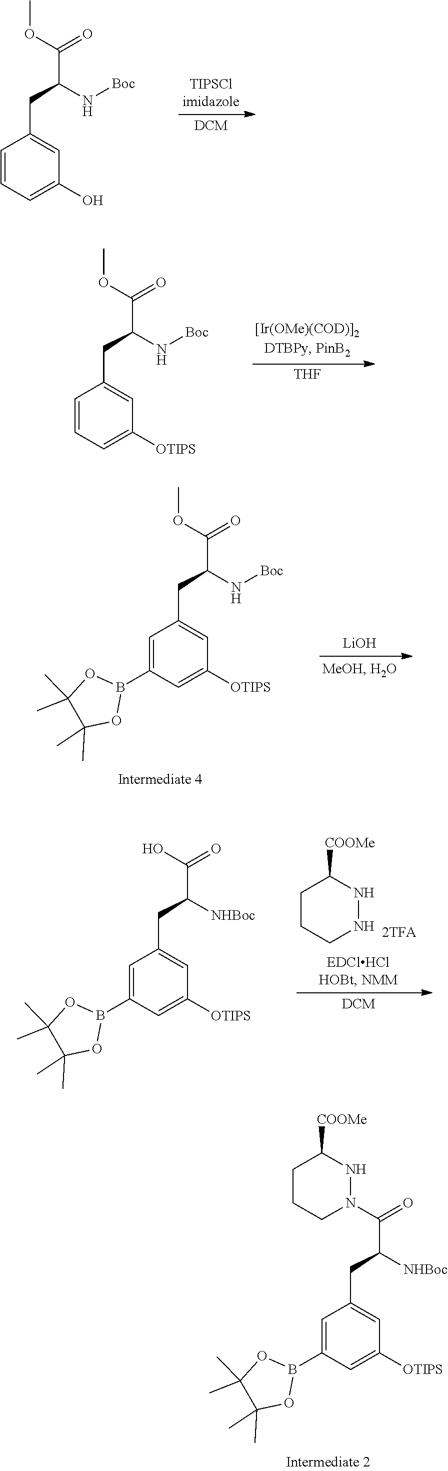

### Step 1: Synthesis of (S)-methyl 2-(tert-butoxycarbonylamino)-3-(3-(triisopropylsilyloxy)phenyl)-propanoate

To a mixture of (S)-methyl 2-(tert-butoxycarbonylamino)-3-(3-hydroxyphenyl)propanoate (10.0 g, 33.9 mmol) in DCM (100 mL) was added imidazole (4.6 g, 67.8 mmol) and TIPSCI (7.8 g, 40.7 mmol). The mixture was stirred at room temperature overnight then diluted with DCM (200 mL) and washed with H₂O (3×150 mL). The organic layer was dried over anhydrous Na₂SO₄, filtered, concentrated under reduced pressure and the residue was purified by silica gel column chromatography to give (S)-methyl 2-(tert-butoxycarbonylamino)-3-(3-(triisopropylsilyloxy)phenyl)-propanoate (15.0 g, 98% yield) as an oil. LCMS (ESI) m/z: [M+Na] calcd for C₂₄H₄₁NO₅SiNa: 474.22; found 474.2.

### Step 2: Synthesis of (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate

A mixture of (S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]₂ (1.1 g, 1.7 mmol) and 4-tert-butyl-2-(4-tert-butyl-2-pyridyl)pyridine (1.3 g, 5.0 mmol) was purged with Ar, then THF (75 mL) was added and the mixture placed under an atmosphere of Ar and sealed. The mixture was heated to 80° C. and stirred for 16 h, concentrated under reduced pressure, and the residue was purified by silica gel column chromatography to give (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate (7.5 g, 78% yield) as a solid. LCMS (ESI) m/z: [M+Na] calcd for C₃₀H₅₂BNO₇SiNa: 600.35; found 600.4; ¹H NMR (300 MHz, CD₃OD) δ 7.18 (s, 1H), 7.11 (s, 1H), 6.85 (s, 1H), 4.34 (m, 1H), 3.68 (s, 3H), 3.08 (m, 1H), 2.86 (m, 1H), 1.41-1.20 (m, 26H), 1.20-1.01 (m, 22H), 0.98-0.79 (m, 4H).

### Step 3: Synthesis of (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid

To a mixture of triisopropylsilyl (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to pH ˜5 with 1M HCl and extracted with EtOAc (2×250 mL). The combined organic layers were washed with brine (3×100 mL), dried over anhydrous Na₂SO₄, filtered and the filtrate concentrated under reduced pressure to give (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (3.7 g, 95% yield), which was used directly in the next step without further purification. LCMS (ESI) m/z: [M+NH₄] calcd for C₂₉H₅₀BNO₇SiNH₄: 581.38; found 581.4.

### Step 4: Synthesis of methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate

To a mixture of methyl (S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (24 g, 42.6 mmol) in DCM (50 mL) then HOBt (1.21 g, 9.0 mmol) and EDCI HCl salt (12.9 g, 67.6 mmol). The mixture was warmed to room temperature and stirred for 16 h, then diluted with DCM (200 mL) and washed with H₂O (3×150 mL). The organic layer was dried over anhydrous Na₂SO₄, filtered, the filtrate concentrated under reduced pressure and the residue was purified by silica gel column chromatography to give methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (22 g, 71/% yield) as an oil. LCMS (ESI) m/z: [M+H] calcd for C₃₅H₆₀BN₃O₈Si: 690.42; found 690.5.

### Intermediate 3. Synthesis of (S)-tert-butyl 3-methyl-2-((S)—N-methylpyrrolidine-3-carboxamido)butanoate

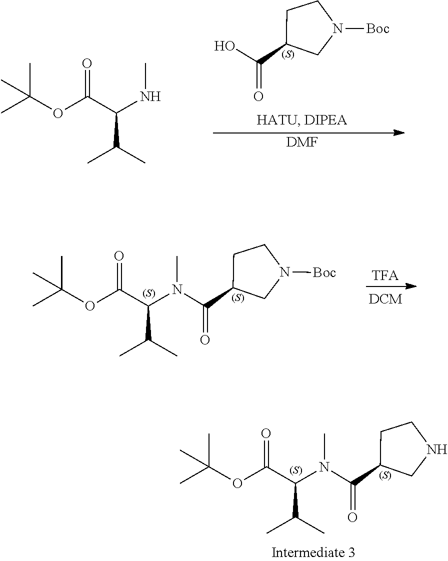

### Step 1: Synthesis of (S)-tert-butyl 3-(((S)-1-(tert-butoxy)-3-methyl-1-oxobutan-2-yl)(methyl)carbamoyl)pyrrolidine-1-carboxylate

To a mixture of (S)-1-(tert-butoxycarbonyl)pyrrolidine-3-carboxylic acid (2.2 g, 10.2 mmol) in DMF (10 mL) at room temperature was added HATU (7.8 g, 20.4 mmol) and DIPEA (5 mL). After stirring at room temperature for 10 min, tert-butyl methyl-L-valinate (3.8 g, 20.4 mmol) in DMF (10 mL) was added. The mixture was stirred at room temperature for 3 h, then diluted with DCM (40 mL) and H₂O (30 mL). The aqueous and organic layers were separated, and the organic layer was washed with H₂O (3×30 mL), brine (30 mL), dried over anhydrous Na₂SO₄ and filtered. The filtrate was concentrated under reduced pressure and the residue was purified by silica gel column chromatography to give (S)-tert-butyl 3-(((S)-1-(tert-butoxy)-3-methyl-1-oxobutan-2-yl)(methyl)carbamoyl)pyrrolidine-1-carboxylate (3.2 g, 82% yield) as an oil. LCMS (ESI) m/z: [M+Na] calcd for C₂₀H₃₆N₂O₅Na: 407.25; found 407.2.

### Step 2: Synthesis of (S)-tert-butyl 3-methyl-2-((S)—N-methylpyrrolidine-3-carboxamido)butanoate

A mixture of (S)-tert-butyl 3-(((S)-1-(tert-butoxy)-3-methyl-1-oxobutan-2-yl)(methyl)carbamoyl)pyrrolidine-1-carboxylate (3.2 g, 8.4 mmol) in DCM (13 mL) and TFA (1.05 g, 9.2 mmol) was stirred at room temperature for 5 h. The mixture was concentrated under reduced pressure to give (S)-tert-butyl 3-methyl-2-((S)—N-methylpyrrolidine-3-carboxamido)butanoate (2.0 g, 84% yield) as an oil. LCMS (ESI) m/z: [M+H] calcd for C₁₅H₂₈N₂O₃: 285.21; found 285.2.

### Intermediate 5. Synthesis of tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

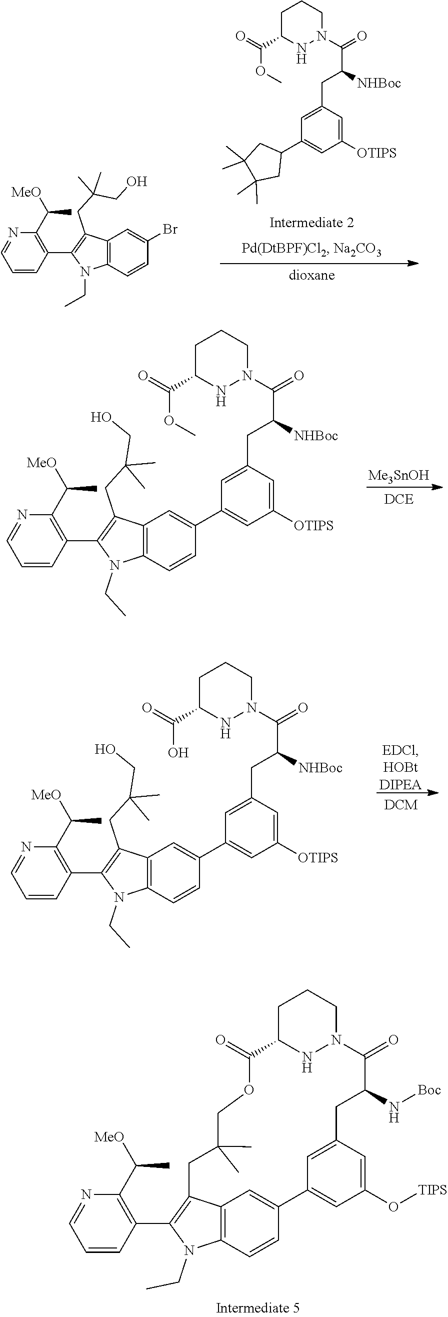

### Step 1: Synthesis of methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate

To a stirred mixture of 3-(5-bromo-1-ethyl-2-[2-[(1S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in 1,4-dioxane (750 mL) at room temperature under an atmosphere of Ar was added Na₂CO₃ (17.9 g, 168.4 mmol), Pd(DtBPF)Cl₂ (4.39 g, 6.7 mmol), and H₂O (150 mL) in portions. The mixture was heated to 85° C. and stirred for 3 h, cooled, diluted with H₂O (2 L), and extracted with EtOAc (3×1 L). The combined organic layers were washed with brine (2×500 mL), dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 72% yield) as a solid. LCMS (ESI) m/z: [M+H] calcd for C₅₂H₇₇N₅O₈Si: 928.56; found 928.8.

### Step 2: Synthesis of (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid

To a stirred mixture of methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at room temperature was added trimethyltin hydroxide (48.7 g, 269 mmol) in portions. The mixture was heated to 65° C. and stirred for 16 h, then filtered and the filter cake washed with DCM (3×150 mL). The filtrate was concentrated under reduced pressure to give (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g, crude), which was used directly in the next step without further purification. LCMS (ESI) m/z: [M+H] calcd for C₅₁H₇₅N₅O₈Si: 914.55; found 914.6.

### Step 3: Synthesis of tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred mixture of (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (400 mL, 2.3 mol), HOBT (51.7 g, 383 mmol) and EDCI (411 g, 2.1 mol) in portions. The mixture was warmed to room temperature and stirred for 16 h, then diluted with DCM (1 L), washed with brine (3×1 L), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (36 g, 42% yield) as a solid. LCMS (ESI) m/z: [M+H] calcd for C₅₁H₇₃N₅O₇Si: 896.54; found 896.5.

### Intermediate 6. Synthesis of tert-butyl ((6³S,4S)-1²-iodo-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate

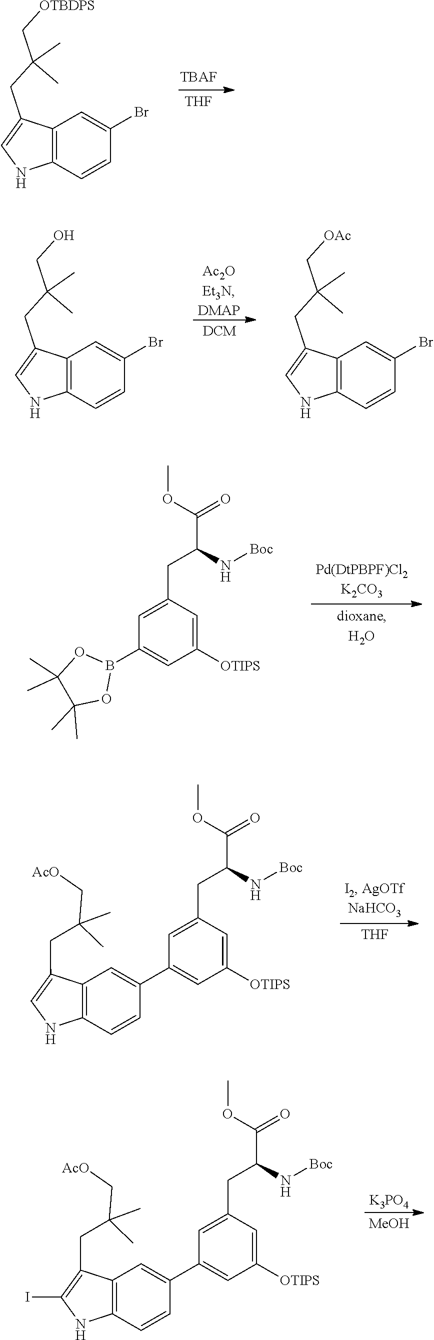

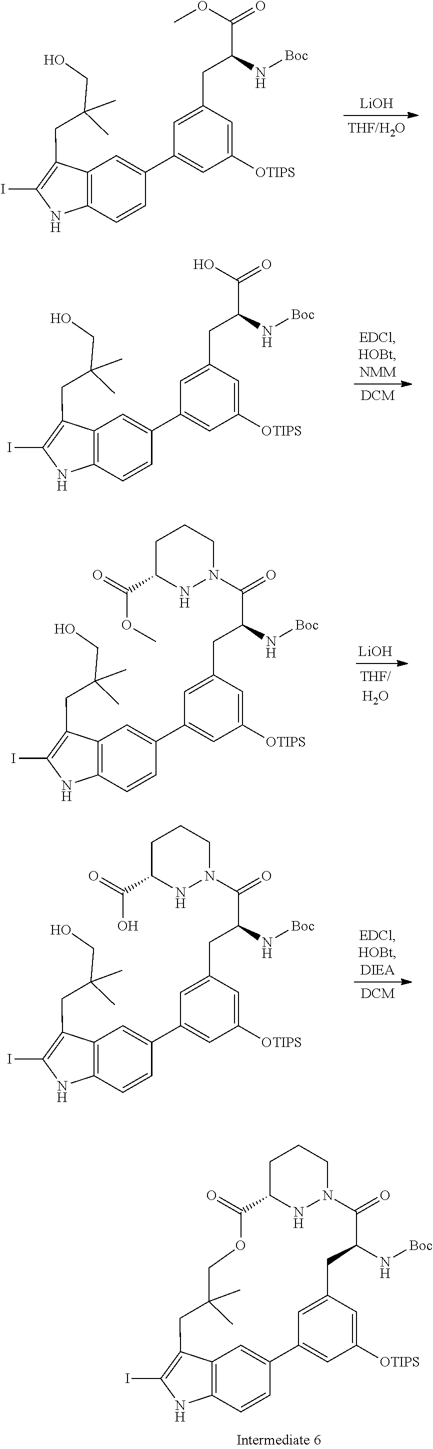

### Step 1: Synthesis of 3-(5-bromo-1H-indol-3-yl)-2,2-dimethylpropan-1-ol

This reaction was undertaken on five batches in parallel on the scale illustrated below. Into a 2 L round-bottom flask were added 5-bromo-3-[3-[(tert-butyidiphenylsilyl)oxy]-2,2-dimethylpropyl]-1H-indole (100 g, 192 mmol) and TBAF (301.4 g, 1.15 mol) in THF (1.15 L) at room temperature. The resulting mixture was heated to 50° C. and stirred for 16 h, then the mixture was concentrated under reduced pressure.

At this stage the residues from all five batches were combined, diluted with H₂O (5 L), and extracted with EtOAc (3×2 L). The combined organic layers were washed with brine (2×1.5 L), dried over anhydrous Na₂SO₄, and filtered. The filtrate was concentrated under reduced pressure and the residue was purified by silica gel column chromatography to give 3-(5-bromo-1H-indol-3-yl)-2,2-dimethylpropan-1-ol (310 g, crude) as a solid. LCMS (ESI) m/z: [M+H] calcd for C₁₃H₁₆BrNO: 282.05 and 284.05; found 282.1 and 284.1.

### Step 2: Synthesis of 3-(5-bromo-1H-indol-3-yl)-2,2-dimethylpropyl acetate

This reaction was undertaken on two batches in parallel in accordance with the procedure below. To a stirred mixture of 3-(5-bromo-1H-indol-3-yl)-2,2-dimethylpropan-1-ol (135 g, 478 mmol) and Et₃N (200 mL, 1.44 mol) in DCM (1.3 L) at 0° C. under an atmosphere of N₂ was added Ac₂O (73.3 g, 718 mmol) and DMAP (4.68 g, 38.3 mmol) in portions. The resulting mixture was stirred for 10 min at 0° C., then washed with H₂O (3×2 L).

At this stage, the organic layers from both batches were combined and washed with brine (2×1 L), dried over anhydrous Na₂SO₄, and filtered. The filtrate was concentrated under reduced pressure and the residue was purified by column chromatography to give 3-(5-bromo-1H-indol-3-yl)-2,2-dimethylpropyl acetate (304 g, 88% yield) as a solid. 1H NMR (400 MHz, DMSO-d₆) δ 11.16-11.11 (m, 1H), 7.69 (d, J=2.0 Hz, 1H), 7.32 (d, J=8.6 Hz, 1H), 7.19-7.12 (m, 2H), 3.69 (s, 2H), 2.64 (s, 2H), 2.09 (s, 3H), 0.90 (s, 6H).

### Step 3: Synthesis of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate

This reaction was undertaken on four batches in parallel in accordance with the procedure below. Into a 2 L round-bottom flasks were added methyl (2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoate (125 g, 216 mmol), 1,4-dioxane (1 L), H₂O (200 mL), 3-(5-bromo-1H-indol-3-yl)-2,2-dimethylpropyl acetate (73.7 g, 227 mmol), K₂CO₃ (59.8 g, 433 mmol), and Pd(DtBPF)Cl₂ (7.05 g, 10.8 mmol) at room temperature under an atmosphere of Ar. The resulting mixture was heated to 65° C. and stirred for 2 h, then diluted with H₂O (10 L) and extracted with EtOAc (3×3 L). The combined organic layers were washed with brine (2×2 L), dried over anhydrous Na₂SO₄, and filtered. The filtrate was concentrated under reduced pressure.

At this point the residue from all four batches was combined and purified by column chromatography to give methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate (500 g, 74% yield) as an oil. LCMS (ESI) m/z: [M+Na] calcd for C₃₉H₅₈N₂O₇SiNa: 717.39; found 717.3.

### Step 4: Synthesis of methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate

This reaction was undertaken on three batches in parallel in accordance with the procedure below. To a stirred mixture of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate (150 g, 216 mmol) and NaHCO₃ (21.76 g, 259 mmol) in THF (1.5 L) was added AgOTf (66.5 g, 259 mmol) in THF dropwise at 0° C. under an atmosphere of nitrogen. 12 (49.3 g, 194 mmol) in THF was added dropwise over 1 h at 0° C. and the resulting mixture was stirred for an additional 10 min at 0° C. The combined experiments were diluted with aqueous Na₂S₂O₃ (5 L) and extracted with EtOAc (3×3 L). The combined organic layers were washed with brine (2×1.5 L), dried over anhydrous Na₂SO₄, filtered, and concentrated under reduced pressure to a residue.

At this stage, the residue from all three batches was combined and purified by column chromatography to give methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate (420 g, 71% yield) as an oil. LCMS (ESI) m/z: [M+Na] calcd for C₃₉H₅₇IN₂O₇SiNa: 843.29; found 842.9.

### Step 5: Synthesis of methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate

This reaction was undertaken on three batches in parallel in accordance with the procedure below. To a 2 L round-bottom flask were added methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate (140 g, 171 mmol), MeOH (1.4 L), and K₃PO₄ (108.6 g, 512 mmol) at 0° C. The mixture was warmed to room temperature and stirred for 1 h, then the combined experiments were diluted with H₂O (9 L) and extracted with EtOAc (3×3 L). The combined organic layers were washed with brine (2×2 L), dried over anhydrous Na₂SO₄, filtered, and the filtrate was concentrated under reduced pressure.

At this stage the residue from all three batches was combined to give methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate (438 g, crude) as a solid. LCMS (ESI) m/z: [M+Na] calcd for C₃₇H₅₅IN₂O₆SiNa: 801.28; found 801.6.

### Step 6: Synthesis of (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid

This reaction was undertaken on three batches in parallel in accordance with the procedure below. To a stirred mixture of methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate (146 g, 188 mmol) in THF (1.46 L) was added LiOH (22.45 g, 937 mmol) in H₂O (937 mL) dropwise at 0° C. The resulting mixture was warmed to room temperature and stirred for 1.5 h [note: LCMS showed 15% de-TIPS product]. The mixture was acidified to pH 5 with 1M HCl (1M) and the combined experiments were extracted with EtOAc (3×3 L). The combined organic layers were washed with brine (2×2 L), dried over anhydrous Na₂SO₄, filtered, and the filtrate was concentrated under reduced pressure.

At this stage the residue from all three batches was combined to give (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid (402 g, crude) as a solid. LCMS (ESI) m/z: [M+Na] calcd for C₃₆H₅₃IN₂O₆SiNa: 787.26; found 787.6.

### Step 7: Synthesis of methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate

To a stirred mixture of (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid (340 g, 445 mmol) and methyl (3S)-1,2-diazinane-3-carboxylate (96.1 g, 667 mmol) in DCM (3.5 L) was added NMM (225 g, 2.2 mol), EDCI (170 g, 889 mmol), and HOBt (12.0 g, 88.9 mmol) portionwise at 0° C. The mixture was warmed to room temperature and stirred for 16 h, then washed with H₂O (3×2.5 L), brine (2×1 L), dried over anhydrous Na₂SO₄, and filtered. The filtrate was concentrated under reduced pressure and the residue was purified by column chromatography to give methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (310 g, 62% yield) as an oil. LCMS (ESI) m/z: [M+H] calcd for C₄₂H₆₃IN₄O₇Si: 891.36; found 890.8.

### Step 8: Synthesis of (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid

This reaction was undertaken on three batches in parallel in accordance with the procedure below. To a stirred mixture of methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (85.0 g, 95.4 mmol) in THF (850 mL) was added LiOH (6.85 g, 286 mmol) in H₂O (410 mL) dropwise at 0° C. under an atmosphere of N₂. The mixture was stirred at 0° C. for 1.5 h [note: LCMS showed 15% de-TIPS product], then acidified to pH 5 with 1M HCl

At this stage the mixtures from all three batches was combined and extracted with EtOAc (3×2 L). The combined organic layers were washed with brine (2×1.5 L), dried over anhydrous Na₂SO₄, filtered, and the filtrate was concentrated under reduced pressure to give (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (240 g, crude) as a solid. LCMS (ESI) m/z: [M+H] calcd for C₄₁H₆₁IN₄O₇Si: 877.35; found 877.6.

### Step 9: Synthesis of tert-butyl ((6³S,4S)-1²-iodo-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate

This reaction was undertaken on two batches in parallel in accordance with the procedure below. To a stirred mixture of (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (120 g, 137 mmol) in DCM (6 L) was added DIPEA (357 mL, 2.05 mol), EDCI (394 g, 2.05 mol), and HOBT (37 g, 274 mmol) in portions at 0° C. under an atmosphere of N₂. The mixture was warmed to room temperature and stirred overnight.

At this stage the solutions from both batches were combined and washed with H₂O (3×6 L), brine (2×6 L), dried over anhydrous Na₂SO₄, and filtered. The filtrate was concentrated under reduced pressure and the residue was purified by column chromatography to give tert-butyl ((6³S,4S)-1²-iodo-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate (140 g, 50% yield) as a solid. LCMS (ESI) m/z: [M+H] calcd for C₄₁H₅₉IN₄O₆Si: 859.33; found 858.3.

### Intermediate 7. Synthesis of (6³S4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

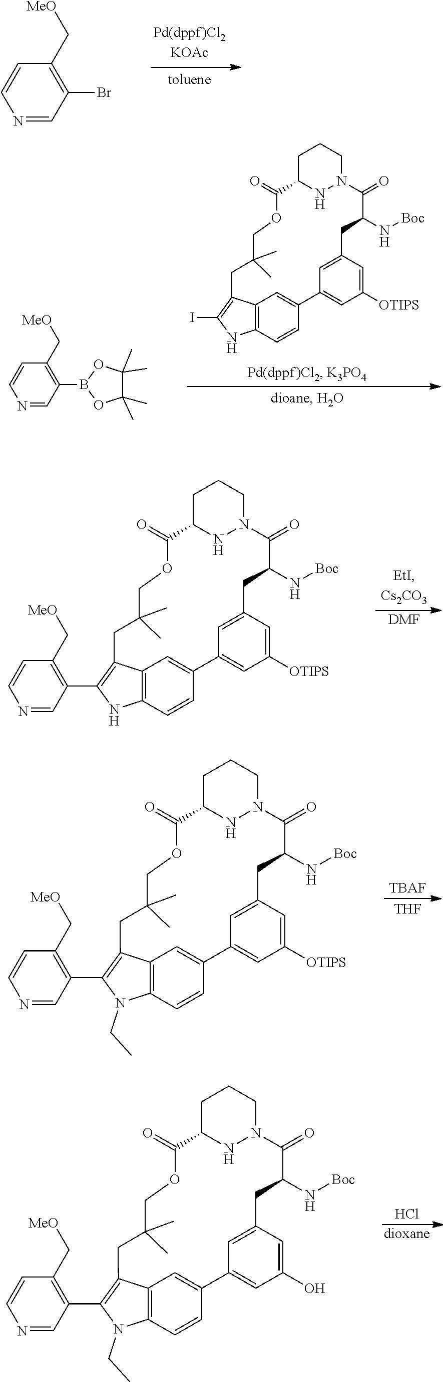

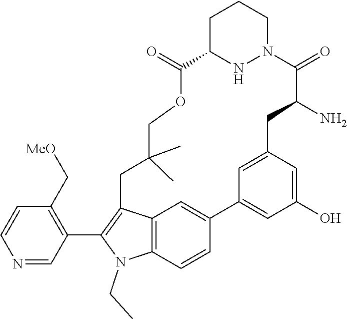 [SMILES: CCn1c(-c2cnccc2COC)c2c3cc(ccc31)-c1cc(O)cc(c1)C[C@H](N)C(=O)N1CCC[C@H](N1)C(=O)OCC(C)(C)C2]

### Step 1: Synthesis of 4-(methoxymethyl)-3-(4,4,5,5-tetramethyl-1,3,2-dioxaborolan-2-yl)pyridine

To a mixture of 3-bromo-4-(methoxymethyl)pyridine (1.0 g, 5.0 mmol), 4,4,5,5-tetramethyl-2-(tetramethyl-1,3,2-dioxaborolan-2-yl)-1,3,2-dioxaborolane (1.51 g, 5.9 mmol) and KOAc (1.21 g, 12.3 mmol) in toluene (10 mL) at room temperature under an atmosphere of Ar was added Pd(dppf)Cl₂ (362 mg, 0.5 mmol). The mixture was heated to 110° C. and stirred overnight, then concentrated under reduced pressure to give 4-(methoxymethyl)-3-(4,4,5,5-tetramethyl-1,3,2-dioxaborolan-2-yl)pyridine, which was used directly in the next step directly without further purification. LCMS (ESI) m/z: [M+H] calcd for C₁₃H₂₀BNO₃: 250.16; found 250.3.

### Step 2: Synthesis of give tert-butyl ((6³S,4S)-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate

To a mixture of 4-(methoxymethyl)-3-(4,4,5,5-tetramethyl-1,3,2-dioxaborolan-2-yl)pyridine (290 mg, 1.16 mmol), K₃PO₄ (371 mg, 1.75 mmol) and tert-butyl ((6³S,4S)-1²-iodo-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate (500 mg, 0.58 mmol) in 1,4-dioxane (5 mL) and H₂O (1 mL) at room temperature under an atmosphere of Ar was added Pd(dppf)Cl₂ (43 mg, 0.06 mmol). The mixture was heated to 70° C. and stirred for 2 h, then H₂O was added and the mixture extracted with EtOAc (2×10 mL). The combined organic layers were washed with brine (10 mL), dried over anhydrous Na₂SO₄, and filtered. The filtrate was concentrated under reduced pressure and the residue was purified by silica gel column chromatography to give tert-butyl ((6³S,4S)-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate (370 mg, 74% yield) as a foam. LCMS (ESI) m/z: [M+H] calcd for C₄₆H₆₇N₅O₇Si: 854.49; found 854.6.

### Step 3: Synthesis of tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate

A mixture of tert-butyl ((6³S,4S)-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate (350 mg, 0.41 mmol), Cs₂CO₃ (267 mg, 0.82 mmol), and EtI (128 mg, 0.82 mmol) in DMF (4 mL) was stirred at 35° C. overnight. H₂O was added and the mixture was extracted with EtOAc (2×15 mL). The combined organic layers were washed with brine (15 mL), dried over anhydrous Na₂SO₄, and filtered. The filtrate was concentrated under reduced pressure and the residue was purified by silica gel column chromatography to give tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate (350 mg, 97% yield) as an oil. LCMS (ESI) m/z: [M+H] calcd for C₅₀H₇₁N₅O₇Si: 882.52; found 882.6.

### Step 4: Synthesis of tert-butyl ((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate

A mixture of tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹-H-8-oxa-1(5,3)-indola-6(1,3)-pyridazina-2(1,3)-benzenacycloundecaphane-4-yl)carbamate (350 mg, 0.4 mmol) and 1M TBAF in THF (0.48 mL, 0.480 mmol) in THF (3 mL) at 0° C. under an atmosphere of Ar was stirred for 1 h. The mixture was concentrated under reduced pressure and the residue was purified by silica gel column chromatography to give tert-butyl ((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-4-yl)carbamate (230 mg, 80% yield) as an oil. LCMS (ESI) m/z: [M+H] calcd for C₄₁H₅₁N₅O₇: 726.39; found 726.6.

### Step 5: Synthesis of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a mixture of tert-butyl N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (200 mg, 0.28 mmol) in 1,4-dioxane (2 mL) at 0° C. under an atmosphere of Ar was added 4M HCl in 1,4-dioxane (2 mL, 8 mmol). The mixture was allowed to warm to room temperature and was stirred overnight, then concentrated under reduced pressure to give (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (200 mg). LCMS (ESI) m/z: [M+H] calcd for C₃₆H₄₃N₅O₅: 626.34; found 626.5.

### Intermediate 8. Synthesis of (6³S,4S)-4-amino-1¹-ethyl-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

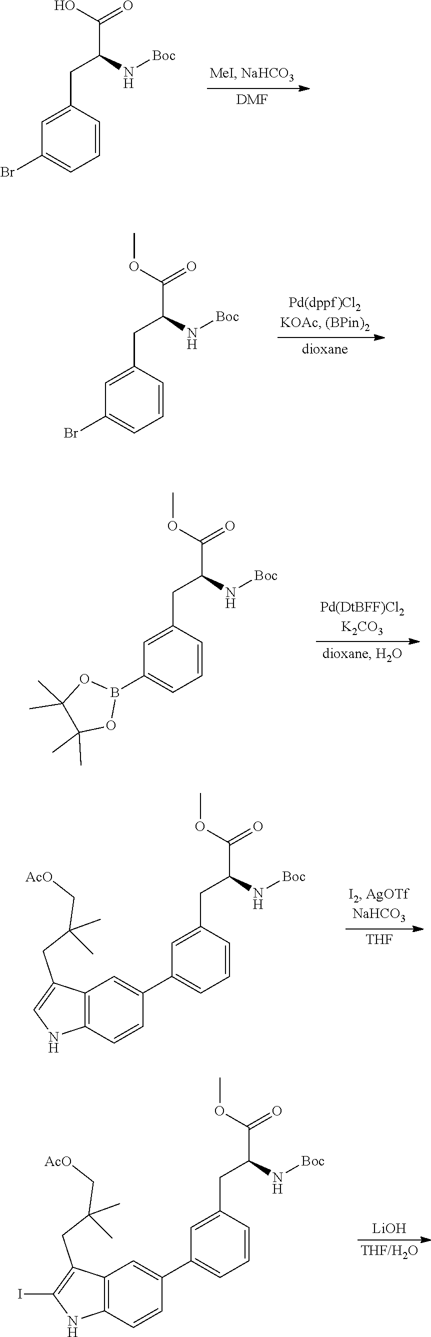

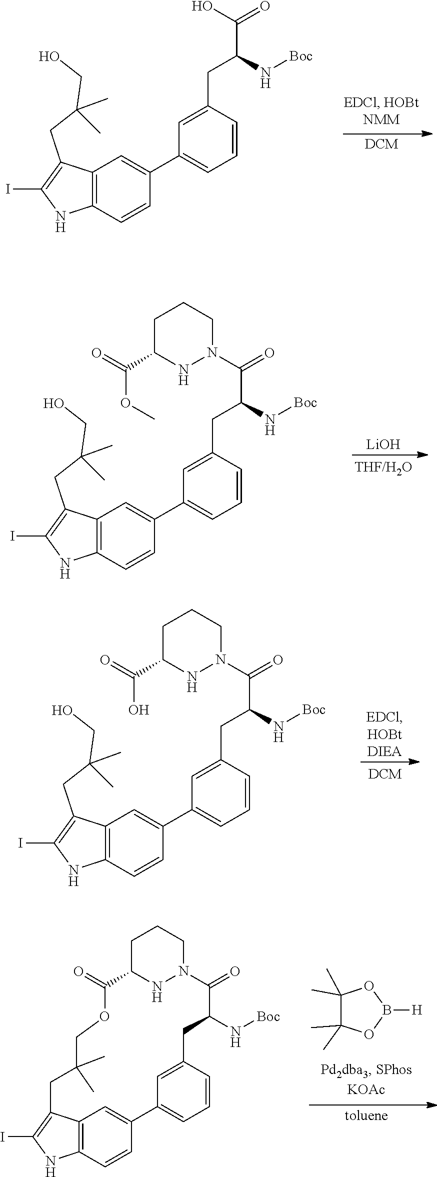

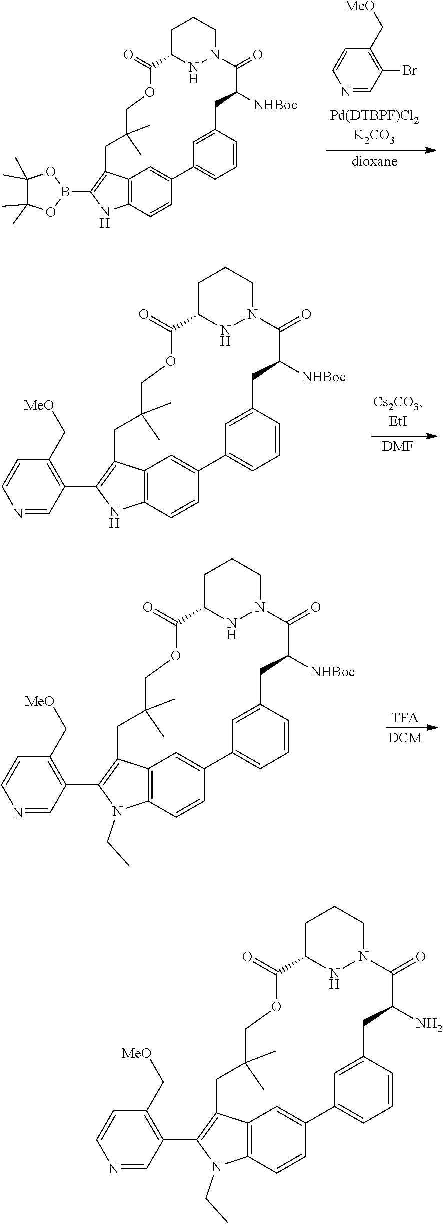

### Step 1: Synthesis of methyl (S)-3-(3-bromophenyl)-2-((tert-butoxycarbonyl)amino)propanoate

To a solution of (2S)-3-(3-bromophenyl)-2-[(tert-butoxycarbonyl)amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel chromatography (13% EtOAc/pet. ether) to afford the desired product (109 g, crude). LCMS (ESI) m/z: [M+Na] calcd for C₁₅H₂₀BrNO₄: 380.05; found 380.0.

### Step 2: Synthesis of methyl (S)-2-((tert-butoxycarbonyl)amino)-3-(3-(4,4,5,5-tetramethyl-1,3,2-dioxaborolan-2-yl)phenyl)propanoate

To a stirred solution of methyl (2S)-3-(3-bromophenyl)-2-[(tert-butoxycarbonyl)amino]propanoate (108 g, 301.5 mmol) and bis(pinacolato)diboron (99.53 g, 391.93 mmol) in 1,4-dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel chromatography (5% EtOAc/pet. ether) to afford the desired product (96 g, 78.6% yield). LCMS (ESI) m/r. [M+Na] calcd for C₂₁H₃₂BNO₆: 428.22; found 428.1.

### Step 3: Synthesis of methyl (S)-3-(3-(3-(3-acetoxy-2,2-dimethylpropyl)-1H-indol-5-yl)phenyl)-2-((tert-butoxycarbonyl)amino)propanoate

To a mixture of methyl (2S)-2-[(tert-butoxycarbonyl)amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in 1,4-dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L) and the combined organic layers were washed with brine (3×600 mL), dried over Na₂SO₄, filtered, and concentrated under reduced pressure. The residue was purified by silica gel chromatography (20% EtOAc/pet. ether) to afford the desired product (130 g, crude). LCMS (ESI) m/z: [M+H] calcd for C₃₀H₃₈N₂O₆: 523.28; found 523.1.

### Step 4: Synthesis of methyl (S)-3-(3-(3-(3-acetoxy-2,2-dimethylpropyl)-2-iodo-1H-indol-5-yl)phenyl)-2-((tert-butoxycarbonyl)amino)propanoate

To a solution of methyl (2S)-3-(3-[3-[3-(acetyloxy)-2,2-dimethylpropyl]-1H-indol-5-yl]phenyl)-2-[(tert-butoxycarbonyl)amino]propanoate (95.0 g, 181.8 mmol) and iodine (36.91 g, 145.41 mmol) in THF (1 L) at −10° C. was added AgOTf (70.0 g, 272.7 mmol) and NaHCO₃ (22.9 g, 272.65 mmol). The reaction mixture was stirred for 30 min and was then quenched by the addition of sat. Na₂S₂O₃ (100 mL) at 0° C. The resulting mixture was extracted with EtOAc (3×1 L) and the combined organic layers were washed with brine (3×500 mL), dried over Na₂SO₄, filtered, and concentrated under reduced pressure. The residue was purified by silica gel chromatography (50% EtOAc/pet. ether) to afford the desired product (49.3 g, 41.8% yield). LCMS (ESI) m/r. [M+H] calcd for C₃₀H₃₇IN₂O₆: 649.18; found 649.1.

### Step 5: Synthesis of (S)-2-((tert-butoxycarbonyl)amino)-3-(3-(3-(3-hydroxy-2,2-dimethylpropyl)-2-iodo-1H-indol-5-yl)phenyl)propanoic acid

To a solution of methyl (2S)-3-(3-[3-[3-(acetyloxy)-2,2-dimethylpropyl]-2-iodo-1H-indol-5-yl]phenyl)-2-[(tert-butoxycarbonyl)amino]propanoate (60 g, 92.5 mmol) in THF (600 mL) was added a solution of LiOH·H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL) and the combined organic layers was washed with sat. brine (2×500 mL), dried over Na₂SO₄, filtered, and concentrated under reduced pressure to afford the desired product (45 g, 82.1% yield). LCMS (ESI) m/z: [M+Na] calcd for C₂₇H₃₃IN₂O₅: 615.13; found 615.1.

### Step 6: Synthesis of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate

To a solution of (2S)-2-[(tert-butoxycarbonyl)amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBT (2.05 g, 15.19 mmol), and EDCI (19.41 g, 101.27 mmol). The reaction mixture was stirred overnight and then the mixture was washed with sat. NH₄Cl (2×200 mL) and sat. brine (2×200 mL), and the mixture was dried over Na₂SO₄, filtered, and concentrated under reduced pressure to afford the desired product (14 g, 38.5% yield). LCMS (ESI) m/z: [M+H] calcd for C₃₁H₄₃IN₄O₆: 718.23; found 719.4.

### Step 7: Synthesis of (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylic acid

To a solution of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate (92 g, 128.0 mmol) in THF (920 mL) at 0° C. was added a solution of LiOH·H₂O (26.86 g, 640.10 mmol) in H₂O (640 mL). The reaction mixture was stirred for 2 h and was then concentrated under reduced pressure to afford the desired product (90 g, crude). LCMS (ESI) m/z: [M+H] calcd for C₃₂H₄₁IN₄O₆: 705.22; found 705.1).

### Step 8: Synthesis of tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of of (3S)-1-[(2S)-2-[(tert-butoxycarbonyl)amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCI (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel chromatography (50% EtOAc/pet. ether) to afford the desired product (70 g, 79.8% yield). LCMS (ESI) m/z: [M+H] calcd for C₃₂H₃₉IN₄O₅: 687.21; found 687.1.

### Step 9: Synthesis of tert-butyl ((6³S,4S)-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((6³S,4S)-12-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22.0 g, 32.0 mmol) in toluene (300.0 mL) was added Pd₂(dba)₃ (3.52 g, 3.85 mmol), S-Phos (3.95 g, 9.61 mmol), and KOAc (9.43 g, 96.13 mmol) followed by 4,4,5,5-tetramethyl-1,3,2-dioxaborolane (26.66 g, 208.3 mmol), dropwise. The resulting solution was heated to 60° C. and stirred for 3 h. The reaction mixture was then cooled to room temperature, filtered, the filter cake was washed with EtOAc, and the filtrate was concentrated under reduced pressure. The residue was purified by normal phase chromatography to afford the desired product (22 g, 90% yield) as a solid. LCMS (ESI) m/z: [M+H] calcd for C₃₈H₅₁BN₄O₇: 687.39; found 687.3.

### Step 10: Synthesis of tert-butyl ((6³S,4S)-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a mixture of tert-butyl ((6³S,4S)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (3.0 g, 4.37 mmol) and 3-bromo-4-(methoxymethyl)pyridine (1.766 g, 8.74 mmol) in dioxane/H₂O (5/1) at 60° C. was added K₂CO₃ (2.415 g, 17.48 mmol) and Pd(DTBPF)Cl₂ (0.5695 g, 0.874 mmol). The reaction mixture was stirred for 4 h. The reaction mixture was cooled to room temperature and was extracted with EtOAc (300 mL). The solution was washed with brine (3×100 mL), dried over Na₂SO₄, filtered, and concentrated under reduced pressure. The residue was purified by normal phase chromatography (50% EtOAc/pet. ether) to afford the desired product (1.96 g, 65.8% yield) as a solid. LCMS (ESI) m/z: [M+H] calcd for C₃₉H₄₇N₅O₆: 682.36; found 682.7.

### Step 11: Synthesis of tert-butyl ((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((6³S,4S)-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.96 g, 2.88 mmol) and ethyl iodide (0.347 mL, 4.31 mmol) in DMF (20.0 mL) was added Cs₂CO₃ (2.342 g, 7.19 mmol). The resulting mixture was stirred at room temperature for 5 h and then diluted with EtOAc (200 mL). The mixture was washed with H₂O (3×100 mL), dried over Na₂SO₄, filtered, and concentrated under reduced pressure. The residue was purified by normal phase chromatography (50% EtOAc/pet. ether) to afford the desired product (1.24 g, 61% yield) as a solid. LCMS (ESI) m/z: [M+H] calcd for C₄₁H₅₁N₅O₆: 710.39; found 710.7.

### Step 12: Synthesis of (6³S,4S)-4-amino-1¹-ethyl-12-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-1¹-ethyl-12-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.09 g, 1.54 mmol) in DCM (1.5 mL) at 0° C. was added TFA (1.50 mL). The reaction mixture was stirred for 1 h, concentrated under reduced pressure, and then azeotroped with toluene (3×20 mL) to afford the desired crude product (1.09 g) as a solid. LCMS (ESI) m/z: [M+H] calcd for C₃₆H₄₃N₅O₄: 610.34; found 610.4.

### Intermediate 9. Synthesis of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl) carbamate

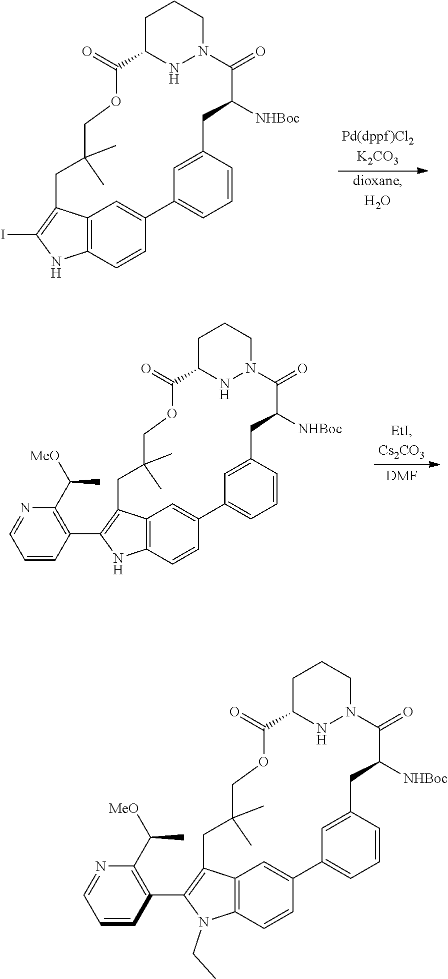

### Step 1: Synthesis of tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl) carbamate

To a solution of tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (13 g, 18.93 mmol) and 2-[(1S)-1-methoxyethyl]-3-(4,4,5,5-tetramethyl-1,3,2-dioxaborolan-2-yl)pyridine (14.95 g, 56.8 mmol) in dioxane (130 mL) and H₂O (26 mL) was added K₂CO₃ (5.23 g, 37.9 mmol) and Pd(dppf)Cl₂ (1.39 g, 1.89 mmol). The reaction mixture was stirred for 4 h at 70° C. The mixture was cooled to room temperature, filtered, and washed with EtOAc (3×100 mL). The filtrate was washed with brine (2×100 mL), dried over Na₂SO₄, filtered, and concentrated under reduced pressure. The residue was purified by prep-TLC (10% MeOH/DCM) to afford the desired product (21 g, 85.3% yield). LCMS (ESI) m/z: [M+H] calcd for C₄₀H₄₉N₅O₆: 696.38; found 696.4.

### Step 2: Synthesis of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl) carbamate

To a solution of tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl) carbamate (20 g, 28.7 mmol) and Cs₂CO₃ (18.7 g, 57.5 mmol) in DMF (150 mL) at 0° C. was added a solution of ethyl iodide (13.45 g, 86.22 mmol) in DMF (50 mL). The resulting mixture was stirred overnight at 35° C. and was then diluted with H₂O (500 mL). The mixture was extracted with EtOAc (2×300 mL) and the combined organic layers were washed with brine (3×100 mL), dried over Na₂SO₄, filtered, and concentrated under reduced pressure. The residue was purified by silica gel chromatography (10%→50% EtOAc/pet. ether) to afford the desired product (4.23 g, 18.8% yield) and the atropisomer (5.78 g, 25.7% yield). LCMS (ESI) m/z: [M+H] calcd for C₄₂H₅₃N₅O₆: 724.41; found 724.4.

### Intermediate 10. Synthesis of (2S)—N((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1H-8-oxa-1(5,3)-indola-6(1,3)-pyridazina-2(1,3)-benzenacycloundecaphane 4-yl)-3-methyl-2-(methylamino)butanamide

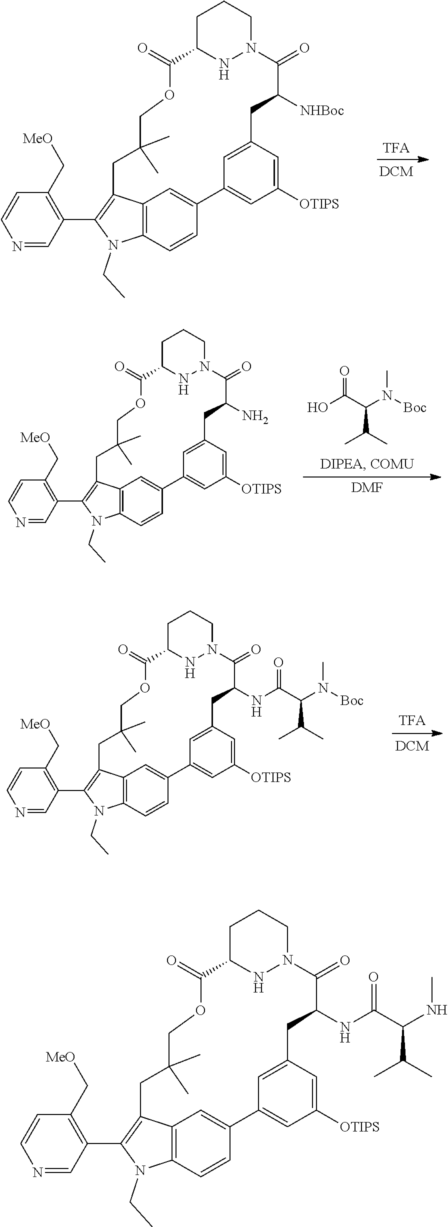

### Step 1: Synthesis of (6³S,4S)-4-amino-1¹-ethyl-1²-(4-(methoxymethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

A mixture of tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (880 mg, 1.2 mmol), DCM (10 mL), and TFA (5 mL) was stirred at 0° C. for 30 min. The mixture was concentrated under reduced pressure to afford the desired product, which was used directly in the next step without further purification. LCMS (ESI) m/z: [M+H] calcd for C₄₅H₆₃N₅O₅Si: 782.47; found 782.7.

### Step 2: Synthesis of tert-butyl ((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate

To a mixture of (6³S,4S)-4-amino-1¹-ethyl-1²-(4-(methoxymethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (880 mg, 1.13 mmol) and N-(tert-butoxycarbonyl)-N-methyl-L-valine (521 mg, 2.3 mmol) in DMF (8.8 mL) at 0° C. was added DIPEA (1.95 mL, 11.3 mmol) and COMU (88 mg, 0.21 mmol). The mixture was stirred at 0° C. for 30 min, then diluted with H₂O (100 mL) and extracted with EtOAc (3×100 mL). The combined organic layers were washed with brine (3×100 mL), dried over anhydrous Na₂SO₄, and filtered. The filtrate was concentrated under reduced pressure and the residue was purified by prep-TLC to afford the desired product (1 g, 89% yield) as a solid. LCMS (ESI) m/z: [M+H] calcd for C₅₆H₈₂N₆O₈Si: 995.61; found 995.5.

### Step 3: Synthesis of (2S)—N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide

A mixture of tert-butyl ((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (1.0 g, 1.0 mmol), DCM (10 mL) and TFA (5 mL) was stirred for 30 min. The mixture was concentrated under reduced pressure and the residue was basified to pH ˜8 with sat. NaHCO₃, then extracted with EtOAc (3×10 mL). The combined organic layers were washed with brine (3×10 mL), dried over Na₂SO₄, filtered, and the filtrate concentrated under reduced pressure to afford the desired product (880 mg, 98% yield) as a solid. LCMS (ESI) m/z: [M+H] calcd for C₅₁H₇₄N₆O₆Si: 895.55; found 895.5.

### Intermediate 11. Synthesis of (2S)—N-((6³S,4S)-1¹-ethyl-2⁵-hydroxy-12(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(N-methyl-2-(methylamino)acetamido)butanamide

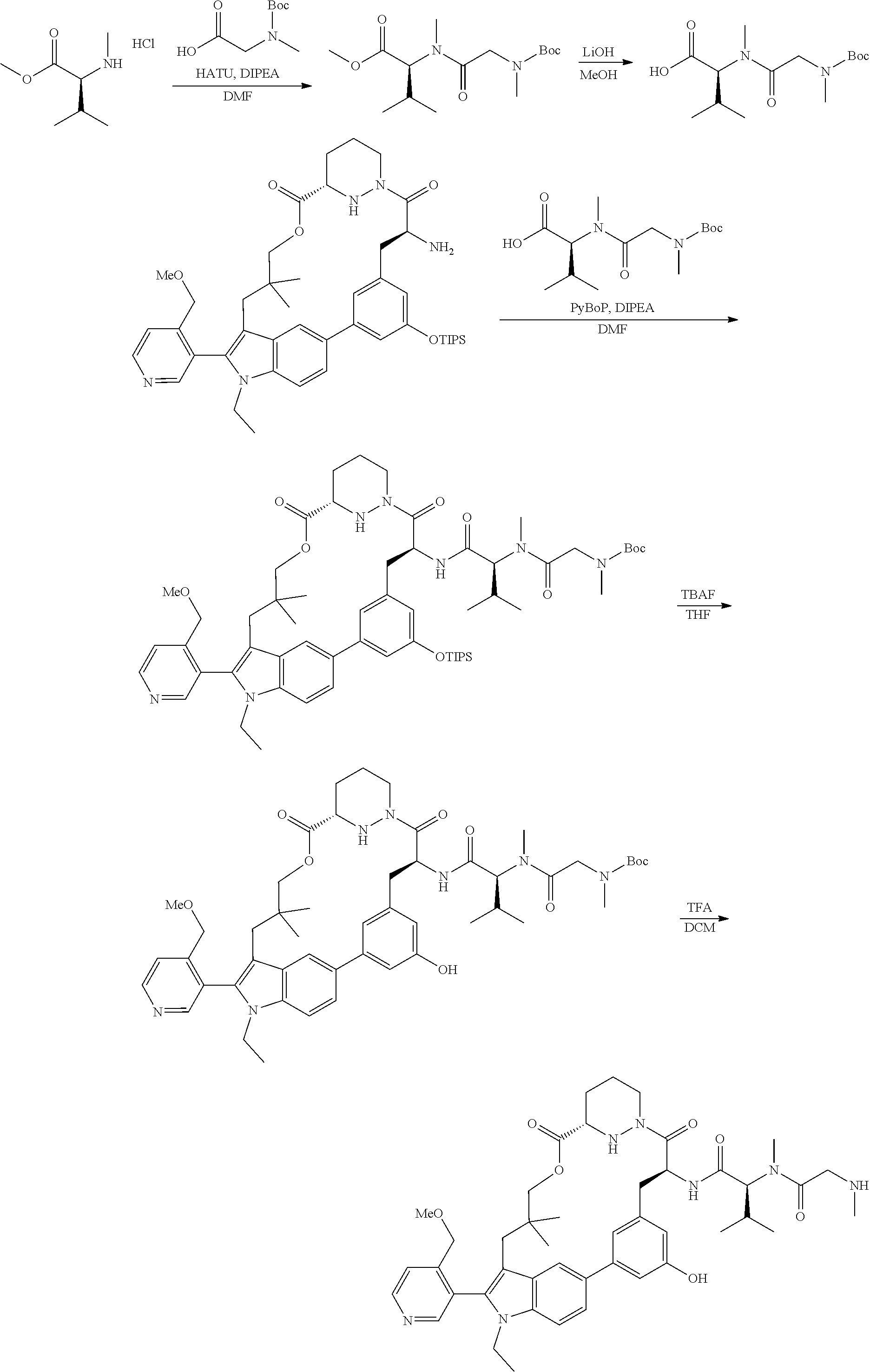

### Step 1: Synthesis of methyl N—(N-(tert-butoxycarbonyl)-N-methylglycyl)-N-methyl-L-valinate

To a solution of methyl methyl-L-valinate hydrochloride (2.0 g, 11.01 mmol) and N-(tert-butoxycarbonyl)-N-methylglycine (3.12 g, 16.51 mmol) in DMF (60.0 mL) at 0° C. was added DIPEA (9.58 mL, 55.01 mmol) and HATU (8.37 g, 22.02 mmol). The reaction mixture was stirred overnight and was then quenched with H₂O (100 mL). The mixture was extracted with EtOAc (3×100 mL) and the combined organic layers were washed with brine (100 mL), dried over Na₂SO₄, and concentrated under reduced pressure. The residue was purified by reverse phase chromatography (40→60% MeCN/H₂O) to afford the desired product (2.9 g, 83% yield) as an oil. LCMS (ESI) m/z: [M+H] calcd for C₁₅H₂₈N₂O₅: 317.21; found 317.4.

### Step 2: Synthesis of N—(N-(tert-butoxycarbonyl)-N-methylglycyl)-N-methyl-L-valine

To a solution of methyl N—(N-(tert-butoxycarbonyl)-N-methylglycyl)-N-methyl-L-valinate (3.70 g, 11.69 mmol) in THF (37.0 mL) was added a solution of LiOH·H₂O (1.96 g, 46.71 mmol) in H₂O (47.0 mL). The reaction mixture was stirred for 4 h, and then 1M HCl was added until the pH was adjusted to 5. The resulting solution was extracted with EtOAc (3×100 mL) and the combined organic layers were washed with brine (3×50 mL), dried over Na₂SO₄, and concentrated under reduced pressure. The residue was purified by reverse phase chromatography (60→60% MeCN/H₂O) to afford the desired product (1.47 g, 41.6% yield) as a solid. LCMS (ESI) m/z: [M+H] calcd for C₁₄H₂₆N₂O₅: 303.19; found 303.4.

### Step 3: Synthesis of tert-butyl (2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate

To a solution of (6³S,4S)-4-amino-1¹-ethyl-1²-(4-(methoxymethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300.0 mg, 0.384 mmol) and N—(N-(tert-butoxycarbonyl)-N-methylglycyl)-N-methyl-L-valine (173.9 mg, 0.575 mmol) in DMF (3.0 mL) at 0° C. was added DIPEA (0.534 mL, 3.069 mmol) and PyBOP (399.2 mg, 0.767 mmol). The reaction mixture was stirred for 2 h and was then diluted with H₂O (30 mL). The resulting mixture was extracted with EtOAc (3×20 mL) and the combined organic layers were washed with brine (3×20 mL), dried over Na₂SO₄, filtered, and concentrated under reduced pressure. The residue was purified by normal phase chromatography (25% EtOAc/pet. ether) to afford the desired product (300 mg, 73% yield) as a solid. LCMS (ESI) m/z: [M+H] calcd for C₅₉H₈₇N₇O₉Si: 1066.64; found 1067.4.

### Step 4: Synthesis of tert-butyl (2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate

To a solution of tert-butyl (2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate (355.0 mg) in THF (4.0 mL) at 0° C. was added TBAF (1.0 mL). The reaction mixture was stirred for 1 h and was then concentrated under reduced pressure. The residue was purified by normal phase chromatography (25% EtOAc/pet. ether) to afford the desired product (280 mg, 92% yield) as a solid. LCMS (ESI) m/z: [M+H] calcd for C₅₀H₆₇N₇O₉: 910.51; found 911.0.

### Step 5: Synthesis of (2S)—N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-(N-methyl-2-(methylamino)acetamido)butanamide

To a solution of tert-butyl (2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate (150.0 mg, 0.165 mmol) in DCM (2.0 mL) at 0° C. was added TFA (0.70 mL). The reaction mixture was stirred for 1 h and was then concentrated under reduced pressure to afford the desired crude product (150 mg) as a solid. LCMS (ESI) m/z: [M+H] calcd for C₄₅H₅₉N₇O₇: 810.46; found 810.4.

### Intermediate 12. Synthesis of (3S)—N((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide

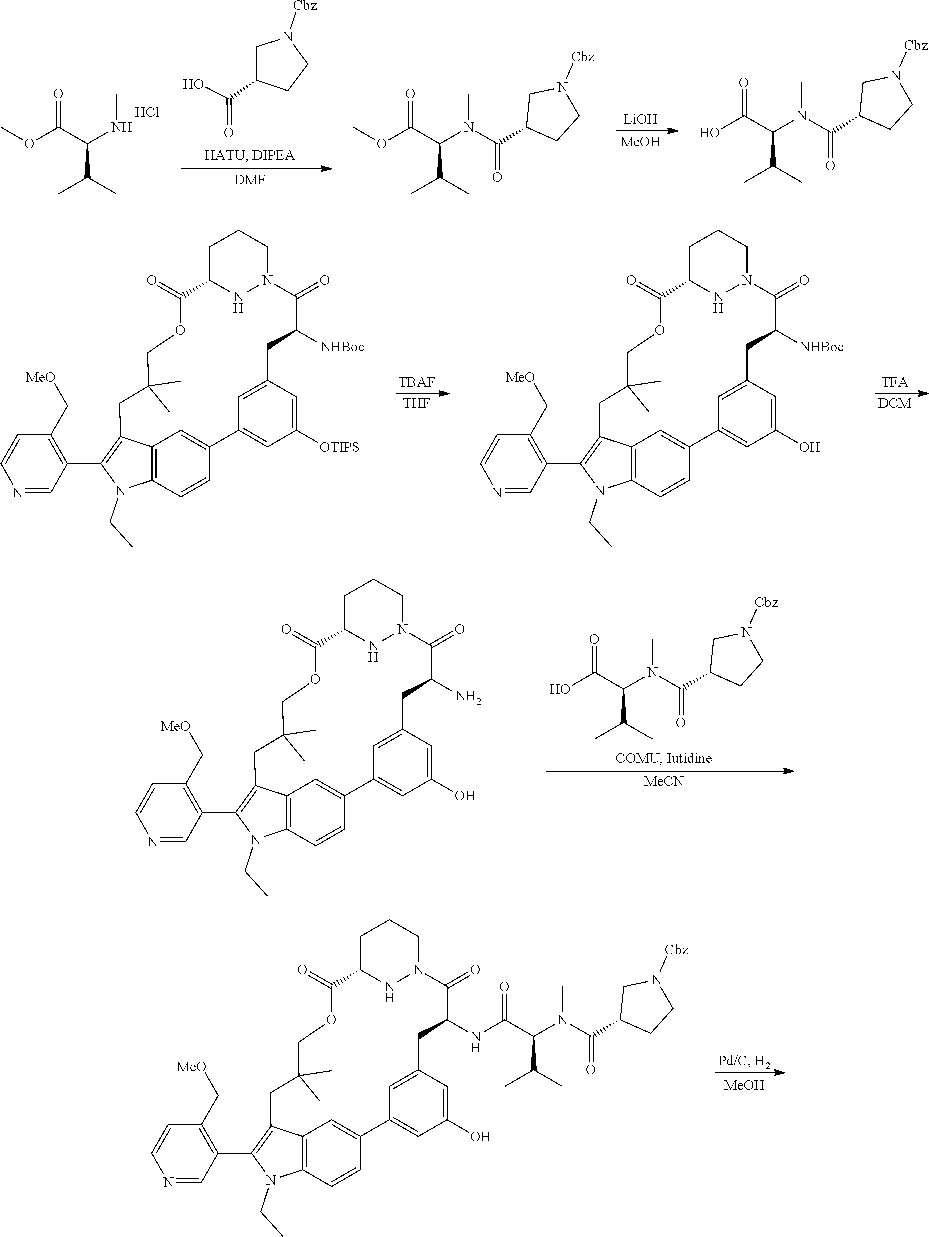

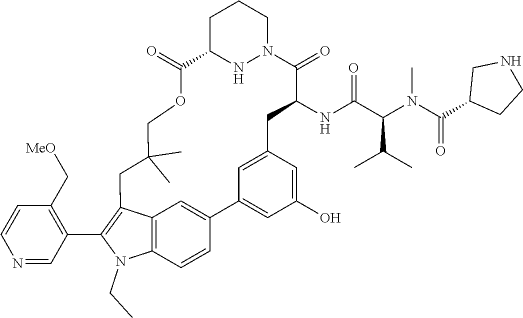 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCNC1)C(=O)N1CCC[C@H](N1)C(=O)OCC(C)(C)C2]

### Step 1: Synthesis of benzyl (S)-3-(((S)-1-methoxy-3-methyl-1-oxobutan-2-yl)(methyl)carbamoyl)pyrrolidine-1-carboxylate

To a solution of methyl methyl-L-valinate hydrochloride (2.0 g, 13.8 mmol) and (S)-1-((benzyloxy)carbonyl)pyrrolidine-3-carboxylic acid (4.12 mg, 16.5 mmol) in DMF (20.0 mL) at 0° C. was added DIPEA (12 mL, 68.870 mmol). The reaction mixture was stirred for 0.5 h, and then HATU (7.856 mg, 20.66 mmol) was added. The resulting mixture was warmed to room temperature and stirred for 1 h. The reaction mixture was then diluted with EtOAc (800 mL) and was washed with sat. NH₄Cl (500 mL) and brine (3×350 mL). The combined organic layers were dried over Na₂SO₄, filtered, and concentrated under reduced pressure. The residue was purified by normal phase chromatography (0→80% EtOAc/pet. ether) to afford the desired product (3.8 g, 73% yield) as an oil. LCMS (ESI) m/z: [M+H] calcd for C₂₀H₂₈N₂O₅: 377.21; found 377.2.

### Step 2: Synthesis of N—((S)-1-((benzyloxy)carbonyl)pyrrolidine-3-carbonyl)-N-methyl-L-valine

To a solution of benzyl (S)-3-(((S)-1-methoxy-3-methyl-1-oxobutan-2-yl)(methyl)carbamoyl)pyrrolidine-1-carboxylate (1.125 g, 2.99 mmol) in MeOH (10.0 mL) was added a solution of LiOH (180.0 mg, 7.52 mmol) in H₂O (2 mL). The reaction mixture was stirred for 4 h and was then quenched with sat. aq. NH₄Cl. The mixture with extracted with EtOAc (3×60 mL) and the combined organic layers were concentrated under reduced pressure to afford the desired product. LCMS (ESI) m/z: [M+H] calcd for C₁₉H₂₆N₂O₅: 363.19; found 363.2.

### Step 3: Synthesis of tert-butyl ((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate

To a solution of tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.70 g, 1.93 mmol) in THF (20 mL) at 0° C. was added TBAF (755.7 mg, 2.89 mmol). The reaction mixture was stirred for 2 h and was then quenched with H₂O (200 mL). The resulting mixture was extracted with EtOAc (3×200 mL) and the combined organic layers were washed with brine (3×200 mL), dried over Na₂SO₄, filtered, and concentrated under reduced pressure. The residue was purified by normal phase chromatography (17% EtOAc/pet. ether) to afford the desired product (1.1 g, 70% yield) as a solid. LCMS (ESI) m/z: [M+H] calcd for C₄₁H₅₁N₅O₇: 726.39; found 726.7.

### Step 4: Synthesis of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (500.0 mg, 0.689 mmol) in DCM (10.0 mL) at 0° C. was added TFA (0.527 mL, 6.888 mmol). The resulting mixture was stirred for 1 h and then was concentrated under reduced pressure to afford the desired crude product (500 mg) as a solid. LCMS (ESI) m/z: [M+H] calcd for C₃₆H₄₃N₅O₅: 626.34; found 626.4.

### Step 5: Synthesis of benzyl (3S)-3-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate

To a solution of N—((S)-1-((benzyloxy)carbonyl)pyrrolidine-3-carbonyl)-N-methyl-L-valine (676.4 mg, 6.31 mmol) in MeCN (10.0 mL) at 0° C. was added COMU (432.5 mg, 1.01 mmol). The reaction mixture was stirred for 5 min followed by the addition of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (395.0 mg, 0.631 mmol). The reaction mixture was warmed to room temperature and stirred for 20 h. The mixture was then concentrated under reduced pressure, taken up in EtOAc (100 mL), and washed with brine (3×5 mL). The organic layer was dried over Na₂SO₄, filtered, and concentrated under reduced pressure. The residue was purified by normal phase chromatography to afford a crude solid (0.81 g), which was then purified by reversed phase chromatography (MeCN/H₂O) to afford the desired product (174 mg, 29% yield) as a solid. LCMS (ESI) m/z: [M+H] calcd for C₅₅H₆₇N₇O: 970.51; found 970.8.

### Step 6: Synthesis of (3S)—N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide

To a solution of benzyl (3S)-3-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate (174.0 mg, 0.179 mmol) in MeOH (20.0 mL) was added Pd/C (87.0 mg, 0.08 mmol) followed by 2% aq. HCl (one drop). The reaction mixture was stirred at room temperature under a H₂ atmosphere (1 atm) for 14 h, at which point the reaction mixture was purged with N₂, filtered, and concentrated under reduced pressure to afford the crude product (130 mg, 86.7% yield) as a solid. LCMS (ESI) m/z: [M+H] calcd for C₄₇H₆₁N₇O₇: 836.47; found 836.5.

### Intermediate 13. Synthesis of (2S)-2-(3-amino-N-methylpropanamido)-N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

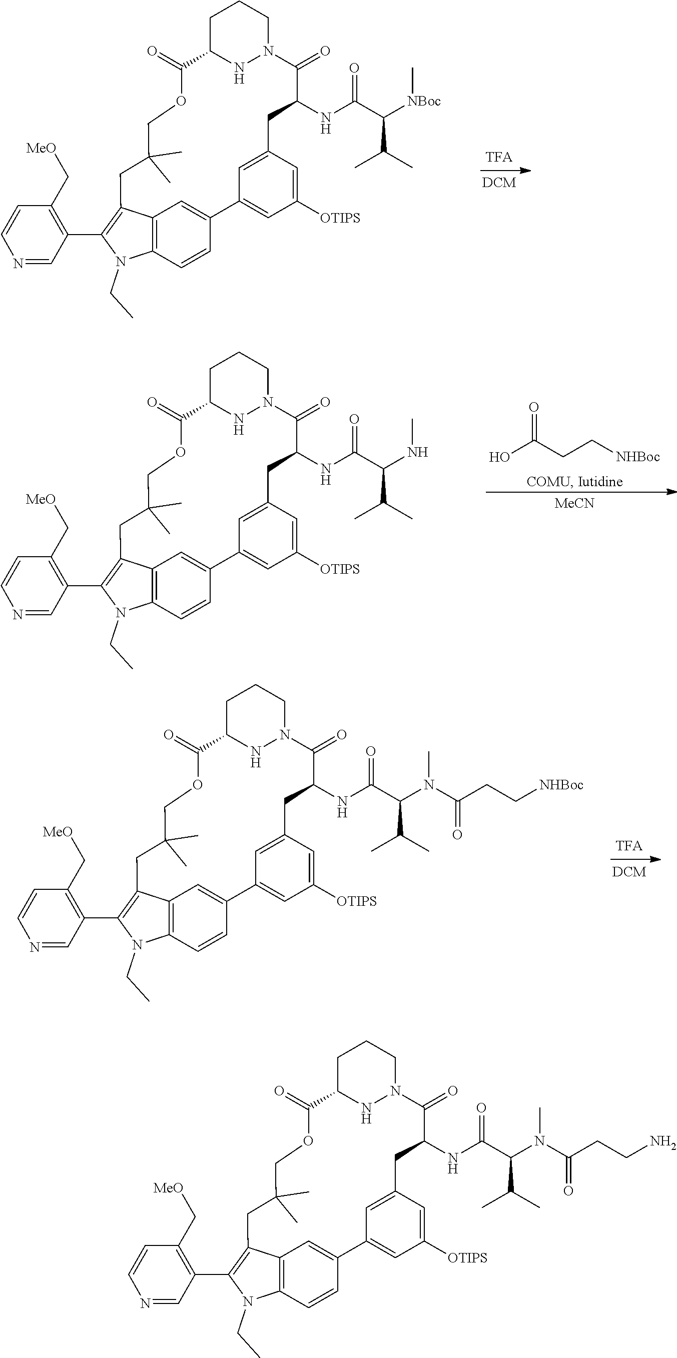

### Step 1: Synthesis of (2S)—N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide

To a solution of tert-butyl ((2S)-1-(((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (212.4 mg, 212 μmol) in DCM (500 μL) at 0° C. was added TFA (500 μL, 6.52 mmol). After 2 h, the reaction was diluted with DCM (10 mL) and H₂O (10 mL), and then sat. aq. NaHCO₃ was added until the solution was pH 9. The aqueous layer was extracted with DCM (10 mL) and the combined organic layers were dried over Na₂SO₄, filtered, and concentrated under reduced pressure to afford the crude product (194 mg, 103% yield). LCMS (ESI) m/z: [M+H] calcd for C₅₁H₇₄N₆O₆Si: 895.55; found 895.7.

### Step 2: Synthesis of tert-butyl (3-(((2S)-1-(((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-3-oxopropyl)carbamate

To a mixture of (2S)—N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (150 mg, 167 μmol), COMU (88.5 mg, 206 μmol), and 3-((tert-butoxycarbonyl)amino)propanoic acid (39.6 mg, 209 μmol) in MeCN (1.66 mL) was added 2,6-lutidine (77.7 μL, 668 μmol). The reaction was stirred for 18 h at room temperature and then for 1 h at 55° C. The reaction mixture was cooled to room temperature and was concentrated under reduced pressure. The crude residue was purified by reverse phase chromatography (20→60% MeCN/H₂O) to afford the product (132 mg, 67% yield). LCMS (ESI) m/z: [M+H] calcd for C₅₉H₈₇N₇O₉Si: 1066.64; found 1066.7.

### Step 3: Synthesis of (2S)-2-(3-amino-N-methylpropanamido)-N-((6³S,4S)-1¹-ethyl-1²-(2-(methoxymethyl)pyridin-3-yl)-10,10-dimethyl-5,7-dioxo-2-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of tert-butyl (3-(((2S)-1-(((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-3-oxopropyl)carbamate (120 mg, 112 μmol) in DCM (560 μL) at 0° C. was added TFA (560 μL, 7.30 mmol). After 40 min, the reaction was diluted with DCM (10 mL) and then sat. aq. NaHCO₃ was added. The organic layer was dried over Na₂SO₄, filtered, and then concentrated under reduced pressure to afford the product (106 mg, 98% yield), which was used in the next step without purification. LCMS (ESI) m/z: [M+H] calcd for C₅₄H₇₉N₇O₇Si: 966.59; found 966.8.

### Intermediate 14. Synthesis of (2S)-2-cyclopentyl-((6³S4S)-1-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5 indola-6(1,3)-pyridazina-2(1,3)-benzenacycloundecaphane-4-yl)-2-(methylamino)acetamide

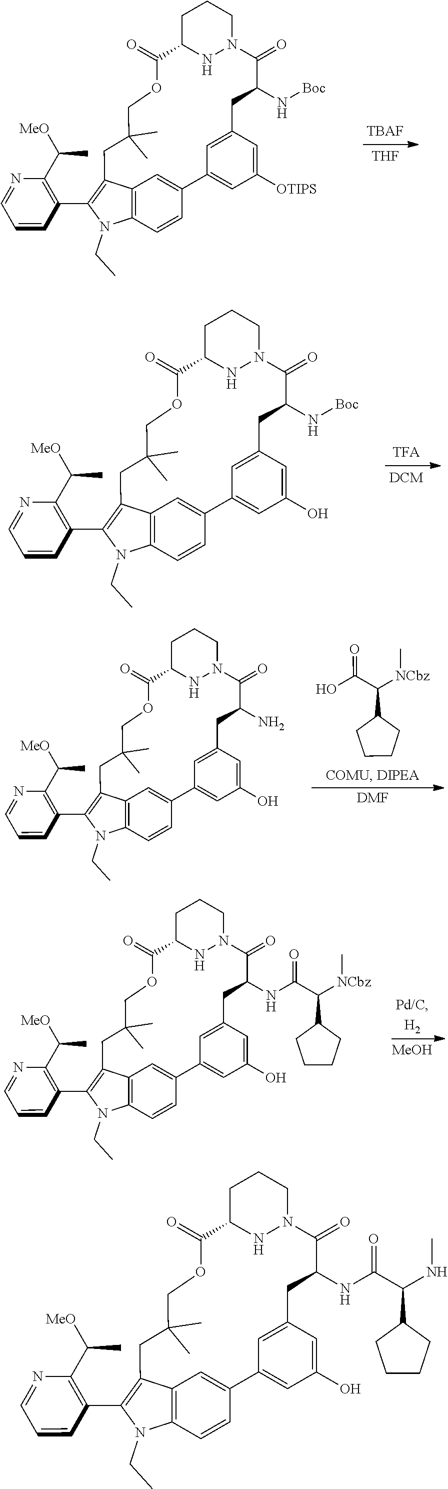

### Step 1: Synthesis of tert-butyl ((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate

To a stirred solution of tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (18.0 g, 20.1 mmol) in THF (180 mL) at 0° C. was added a 1M solution of TBAF in THF (24.1 mL, 24.1 mmol). The mixture was stirred at 0° C. for 1 h, then diluted with brine (1.5 L), and extracted with EtOAc (3×1 L). The combined organic layers were washed with brine (2×500 mL), dried over anhydrous Na₂SO₄, filtered, and concentrated under reduced pressure. Purification by normal phase chromatography afforded the desired product (11.5 g, 69% yield). LCMS (ESI) m/z: [M+H] calcd for C₄₂H₅₃N₅O₇: 740.40; found 740.4.

### Step 2: Synthesis of (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred solution of tert-butyl ((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (11.5 g, 15.5 mmol) in DCM (120 mL) at 0° C. was added TFA (60 mL, 808 mmol). The mixture was stirred at 0° C. for 1 h, then concentrated under reduced pressure and the residue again concentrated under reduced pressure with toluene (3×20 mL) to afford the desired crude product (12 g). LCMS (ESI) m/z: [M+H] calcd for C₃₇H₄₅N₅O₅: 640.35; found 640.6.

### Step 3: Synthesis of benzyl ((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamate

To a stirred solution of (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (400.0 mg, 0.63 mmol) in DMF (4.0 mL) at 0° C. was added DIPEA (1.09 mL, 6.25 mmol) and (S)-2-(((benzyloxy)carbonyl)(methyl)amino)-2-cyclopentylacetic acid (255.0 mg, 0.88 mmol) followed by COMU (347.8 mg, 0.81 mmol). The resulting mixture was stirred at 0° C. for 1 h and was then diluted with H₂O (40 mL). The aqueous layer was extracted with EtOAc (3×15 mL) and the combined organic layers were washed with brine (2×10 mL), dried over Na₂SO₄, filtered, and concentrated under reduced pressure. The residue was purified by prep-TLC (25% EtOAc/pet. ether) to afford the desired product (510 mg, 80% yield). LCMS (ESI) m/z: [M+H] calcd for C₅₃H₆₄N₆O₅: 913.49; found 913.6.

### Step 4: Synthesis of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(methylamino)acetamide

To a stirred solution of benzyl ((1S)-1-cyclopentyl-2-(((6³S,4S)-1¹-ethyl-2-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamate (480.0 mg, 0.53 mmol), in MeOH (25 mL) was added Pd/C (200.0 mg, 1.88 mmol). The resulting mixture was placed under an atmosphere of H₂ (1 atm) and stirred for 2 h. The mixture was filtered, the filter cake was washed with MeOH (3×10 mL), and the filtrate was concentrated under reduced pressure to afford the desired crude product (440 mg). LCMS (ESI) m/z. [M+H] calcd for C₄₅H₅₆N₆O₆: 779.45; found 779.4.

### Intermediate 15. Synthesis of (2S)—N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1H-8-oxa-1(5,3)-indola-6(1,3)-pyridazina-2(1,3)-benzenacycloundecaphane-4-yl)-3-methyl-2-(N-methyl-3-(methylamino)propanamido)butanamide

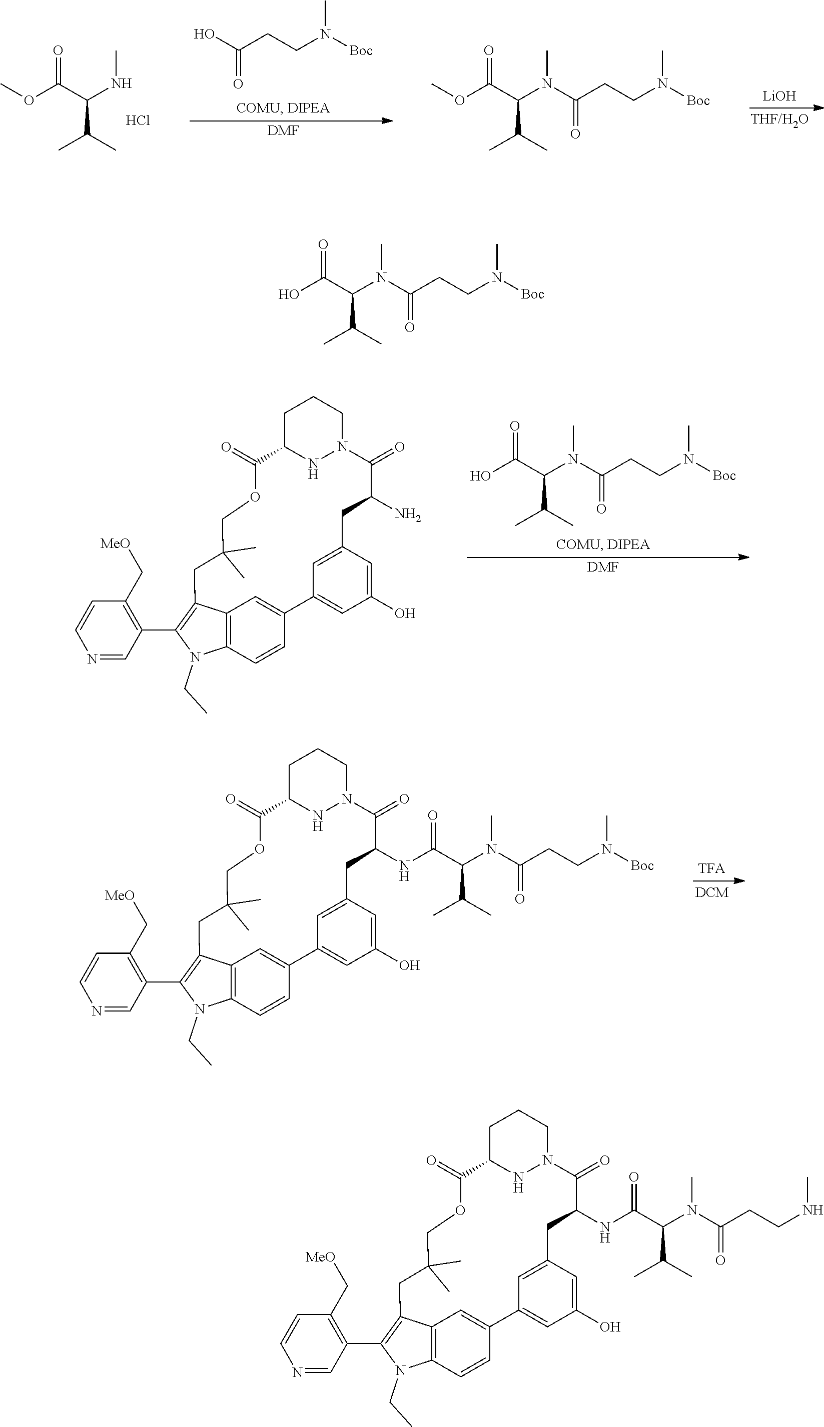

### Step 1: Synthesis of methyl N-(3-((tert-butoxycarbonyl)(methyl)amino)propanoyl)-N-methyl-L-valinate

To a solution of methyl methyl-L-valinate hydrochloride (1.0 g, 6.89 mmol) in DMF (20.0 mL) at 0° C. was added DIPEA (5.92 mL, 0.034 mmol), 3-((tert-butoxycarbonyl)(methyl)amino)propanoic acid (2.10 g, 0.010 mmol), and COMU (3.54 g, 8.27 mmol). The resulting mixture was stirred for 30 min and then quenched with H₂O (20 mL). The aqueous layer was extracted with EtOAc (3×20 mL) and the combined organic layers were washed with brine (3×20 mL), dried over Na₂SO₄, filtered, and concentrated under reduced pressure. The residue was purified by reverse phase chromatography (0→100% MeCN/H₂O) to afford the desired product (2 g, 87.9% yield). LCMS (ESI) m/z: [M+H] calcd for C₁₆H₃₀N₂O₅: 331.22; found 331.2.

### Step 2: Synthesis of N-(3-((tert-butoxycarbonyl)(methyl)amino)propanoyl)-N-methyl-L-valine

To a solution of N-(3-((tert-butoxycarbonyl)(methyl)amino)propanoyl)-N-methyl-L-valinate (1.0 g, 3.03 mmol) in THF (20.0 mL) and H₂O (4.0 mL) was added LiOH (0.14 g, 6.05 mmol). The resulting mixture was stirred for 3 h at room temperature. The mixture was acidified to pH 3 with HCl (1N) and was then extracted with EtOAc (3×20 mL). The combined organic layers were washed with brine (3×20 mL), dried over Na₂SO₄, filtered, and concentrated under reduced pressure to afford the desired crude product (800 mg, 83.6% yield). LCMS (ESI) m/z: [M+H] calcd for C₁₅H₂₈N₂O₅: 317.21; found 317.2.

### Step 3: Synthesis of tert-butyl (3-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-3-oxopropyl)(methyl)carbamate

To a solution of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (600.0 mg, 0.96 mmol) in DMF (6.0 mL) at 0° C. was added DIPEA (1.67 mL, 9.59 mmol), N-(3-((tert-butoxycarbonyl)(methyl)amino)propanoyl)-N-methyl-L-valine (455.1 mg, 1.44 mmol), and COMU (492.5 mg, 1.15 mmol). The resulting mixture was stirred for 30 min and was then quenched with H₂O (60 mL). The aqueous layer was extracted with EtOAc (3×60 mL) and the combined organic layers were washed with brine (3×60 mL), dried over Na₂SO₄, filtered, and concentrated under reduced pressure. The residue was purified by reverse phase chromatography (0→100% MeCN/H₂O) to afford the desired product (650 mg, 73.4% yield). LCMS (ESI) m/z: [M+H] calcd for C₅₁H₆₉N₇O₉: 924.52; found 924.6.

### Step 4: Synthesis of (2S)—N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-(N-methyl-3-(methylamino)propanamido)butanamide

To a solution of tert-butyl (3-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-3-oxopropyl)(methyl)carbamate (650.0 mg) in DCM (7.0 mL) at 0° C. was added TFA (3.5 mL). The resulting mixture was stirred for 30 min and was then concentrated under reduced pressure. The resulting residue was diluted with toluene (3×10 mL) and concentrated under reduced pressure to afford the desired crude product. LCMS (ESI) m/z: [M+H] calcd for C₄₆H₆₁N₇O₇: 824.47; found 824.6.

### Intermediate 16. Synthesis of (2S)-2-cyclopentyl-((6³S4S)-1-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(methyl-2-(methylamino)acetamido)acetamide

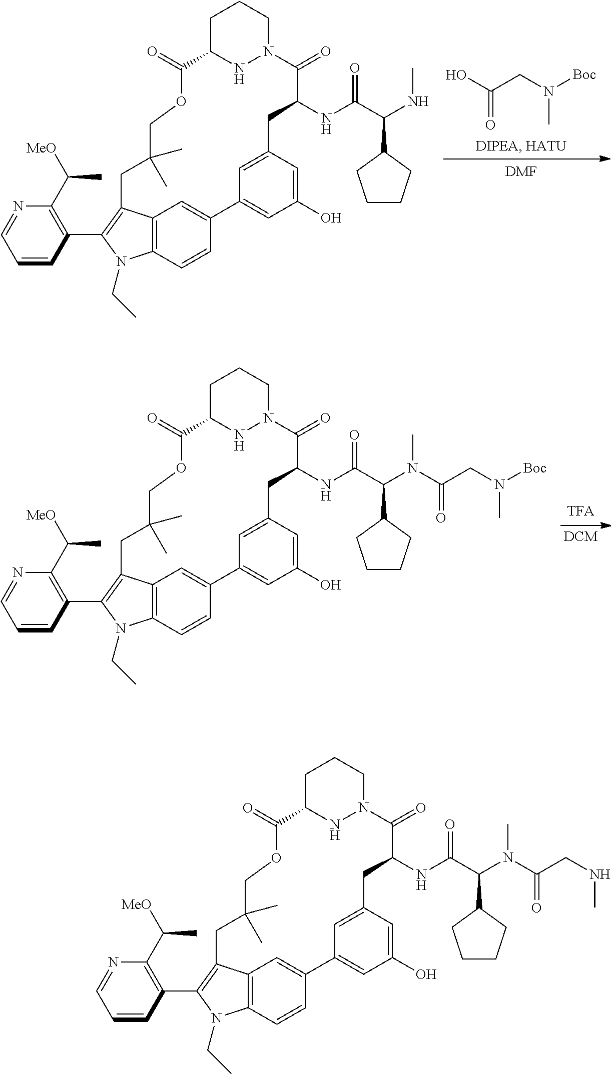

### Step 1: Synthesis of tert-butyl (2-(((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)(methyl)carbamate

To a mixture of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(methylamino)acetamide (300.0 mg, 0.385 mmol), DIPEA (0.657 mL, 3.851 mmol), and N-(tert-butoxycarbonyl)-N-methylglycine (109.30 mg, 0.578) in DMF (3.0 mL) at 0° C. was added HATU (175.72 mg, 0.462 mmol). The resulting mixture was stirred at 0° C. for 30 min and was then diluted with H₂O (30 mL). The resulting mixture was extracted with EtOAc (3×30 mL). The combined organic layers were washed with brine (3×30 mL), dried with Na₂SO₄, filtered, and concentrated under reduced pressure. Purification by prep-TLC (50% EtOAc/pet. ether) to afford the desired product (300 mg, 82.0% yield). LCMS (ESI) m/z: [M+H] calcd for C₅₃H₇₁N₇O₉: 950.54; found 950.4.

### Step 2: Synthesis of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(N-methyl-2-(methylamino)acetamido)acetamide

To a mixture of tert-butyl (2-(((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)(methyl)carbamate (300.0 mg, 0.316 mmol) in DCM (3.0 mL) at 0° C. was added TFA (1.50 mL). The resulting mixture was stirred at 0° C. for 30 min and was then concentrated under reduced pressure to afford the desired crude product. LCMS (ESI) m/z: [M+H] calcd for C₄₈H₆₃N₇O₇: 850.49; found 850.5.

### Intermediate 17. Synthesis of (2R,5R)—N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N,5-dimethylpyrrolidine-2-carboxamide

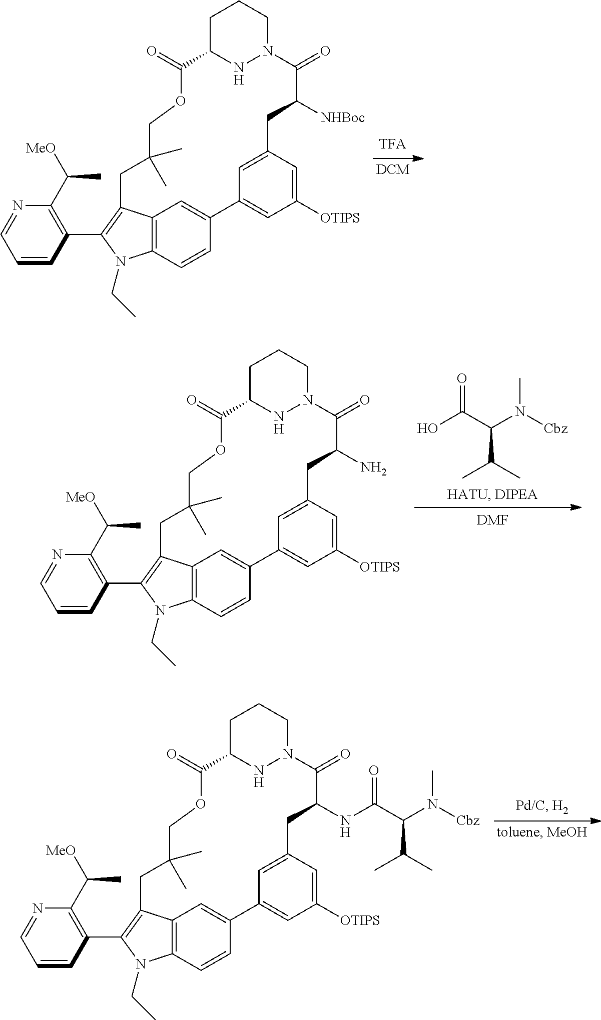

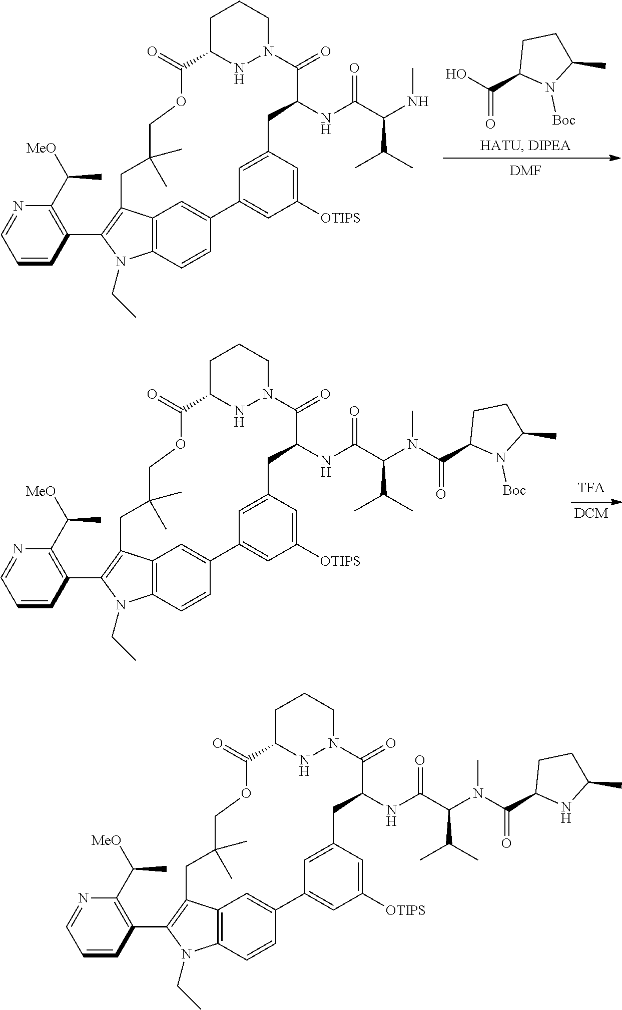

### Step 1: Synthesis of (6³S,4S)-4-amino-1¹-ethyl-1²-(2-((S)-1-methoxyethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (20.0 g, 22.315 mmol) in DCM (150.0 mL) at 0° C. was added TFA (50.0 mL). The resulting mixture was warmed to room temperature and stirred for 2 h and then concentrated under reduced pressure. The residue was dissolved in EtOAc (100 mL) and the solution was neutralized to pH 8 with sat. aq. NaHCO₃. The solution was extracted with EtOAc (3×150 mL) and the combined organic layers were washed with brine (100 mL), dried over Na₂SO₄, filtered, and concentrated under reduced pressure to afford the desired product (17.86 g, crude). LCMS (ESI) m/z: [M+H] calcd for C₄₆H₆₅N₅O₅Si: 796.49; found 795.5.

### Step 2: Synthesis of benzyl ((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate

To a solution of (6³S,4S)-4-amino-1¹-ethyl-1²-(2-((S)-1-methoxyethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)- benzenacycloundecaphane-5,7-dione (17.86 g, 22.433 mmol) and (2S)-2-[[(benzyloxy)carbonyl](methyl)amino]-3-methylbutanoic acid (8.93 g, 33.65 mmol) in DMF (150.0 mL) at 0° C. was added DIPEA (19.5 mL, 112.17 mmol) and HATU (17.06 g, 44.87 mmol). The resulting mixture was warmed to room temperature and stirred for 2 h. The reaction mixture was cooled to 0° C. and was quenched by the addition of H₂O (500 mL). The mixture was extracted with EtOAc (3×150 mL) and the combined organic layers were washed with brine (200 mL), dried over Na₂SO₄, filtered, and concentrated under reduced pressure. The residue was purified by normal phase chromatography (25% EtOAc/pet. ether) to afford the desired product (19.0 g, 81.2% yield). LCMS (ESI) m/z: [M+H] calcd for C₆₀H₈₂N₆O₈Si: 1043.61; found 1042.6.

### Step 3: Synthesis of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide

To a solution of benzyl ((2S)-1-(((6³S,4S)-1I-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (1.20 g, 1.150 mmol) in MeOH (1.2 mL) and toluene (1.2 mL) was added Pd/C (10%, 240 mg). The resulting mixture was placed under an atmosphere of H₂ (1 atm) and stirred overnight. The mixture was filtered and concentrated under reduced pressure to afford the desired product (1.05 g, 97.4% yield). LCMS (ESI) m/z: [M+H] calcd for C₅₂H₇₆N₆O₆Si: 909.57; found 909.3.

### Step 4: Synthesis of tert-butyl (2R,5R)-2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-5-methylpyrrolidine-1-carboxylate

To a solution of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (500 mg, 0.550 mmol) in DMF (5 mL) at 0° C. was added DIPEA (0.94 mL, 5.499 mmol) and (2R,5R)-1-(tert-butoxycarbonyl)-5-methylpyrrolidine-2-carboxylic acid (504.29 mg, 2.199 mmol) followed by HATU (627.23 mg, 1.650 mmol) in portions. The resulting mixture was warmed to room temperature and stirred for 1 h. Purification by reverse phase chromatography (0→100% MeCN/H₂O) afforded the desired product (147 mg, 22.2% yield). LCMS (ESI) m/z: [M+H] calcd for C₆₃H₉₃N₇O₉Si: 1120.69; found 1120.6.

### Step 5: Synthesis of (2R,5R)—N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N,5-dimethylpyrrolidine-2-carboxamide

To a solution of tert-butyl (2R,5R)-2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-5-methylpyrrolidine-1-carboxylate (150.0 mg, 0.134 mmol) in DCM at 0° C. was added TFA (1.50 mL, 13.155 mmol) dropwise. The resulting mixture was warmed to room temperature and stirred for 2 h and was then basified to pH 8 with sat. NaHCO₃. The resulting mixture was extracted with EtOAc (3×5 mL) and the combined organic layers were washed with brine (2×5 mL), dried with Na₂SO₄, filtered, and concentrated under reduced pressure to afford the desired product (85 mg, 54.1% yield). LCMS (ESI) m/z: [M+H] calcd for C₅₈H₈₅N₇O₇Si: 1020.64; found 1020.4.

### Intermediate 18. Synthesis of (2R)—N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-i-methylpyrrolidine-2-carboxamide

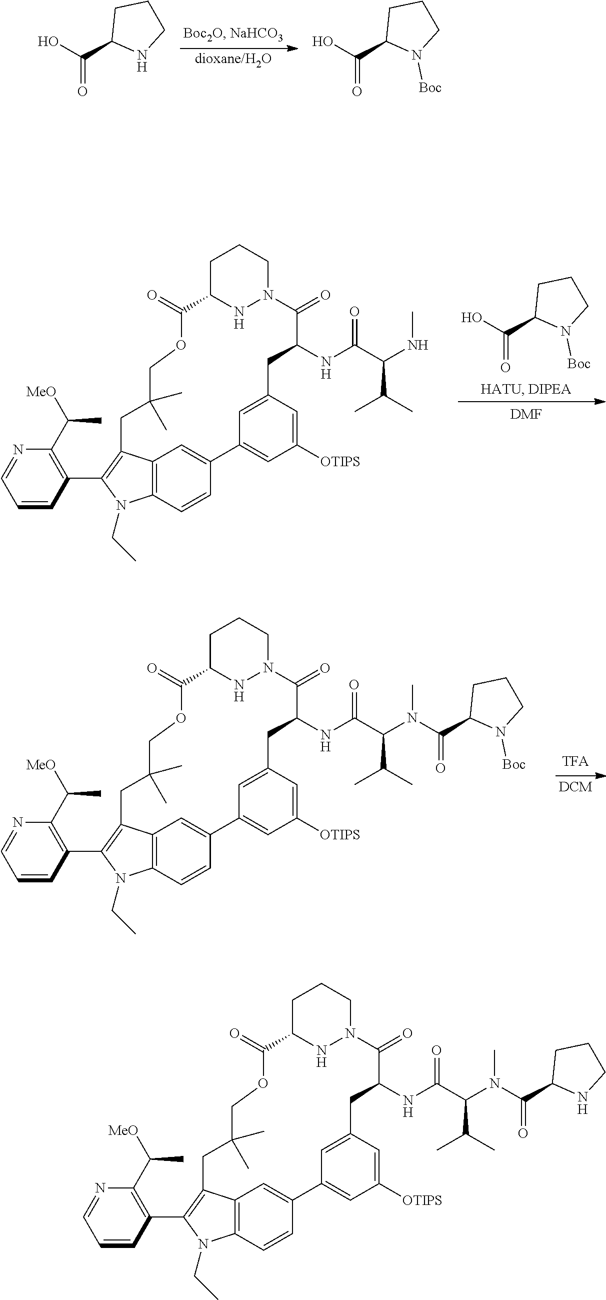

### Step 1: Synthesis of (tert-butoxycarbonyl)-D-proline

To a solution of D-proline (5.0 g, 43.43 mmol) in 1,4-dioxane (50 mL) and sat. NaHCO₃ (50 mL) at 0° C. was added Boc₂O (14.217 g, 65.143 mmol) in portions. The resulting mixture was stirred for 2 h at room temperature and was then extracted with EtOAc (100 mL). The aqueous layer was acidified to pH 6 with HCl and was then extracted into EtOAc (3×100 mL). The combined organic layers were washed with H₂O (2×100 mL), dried with Na₂SO₄, filtered, and concentrated under reduced pressure to afford the desired product which was used without further purification. LCMS (ESI) m/z: [M−H] calcd for C₁₀H₁₇NO₄: 214.11; found 214.0.

### Step 2: Synthesis of tert-butyl (2R)-2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate

To a solution of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (142.03 mg, 0.660 mmol) in DMF was added DIPEA (0.710 mL, 5.499 mmol) followed by HATU (250.89 mg, 0.660 mmol) in portions. The resulting mixture was heated to 40° C. and stirred for 2 h. Purification by reverse phase chromatography (0→100% MeCN/H₂O) afforded the desired product (350 mg, 54.6% yield). LCMS (ESI) m/z: [M+H] calcd for C₆₂H₉₁N₇O₉Si: 1106.67; found 1106.8.

### Step 3: Synthesis of (2R)—N-((2S)-1-(((6³S,4S)-1¹-ethyl-1²-(2-((5)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-i-methylpyrrolidine-2-carboxamide

To a solution of tert-butyl (2R)-2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate (350.0 mg, 0.325 mmol) in DCM (4 mL) at 0° C. was added TFA (2.0 mL). The resulting mixture was stirred for 30 min at 0° C. and then was concentrated under reduced pressure. The residue was dissolved in toluene (5 mL) then concentrated under reduced pressure three times to afford the desired product which was used without further purification. LCMS (ESI) m/z: [M+H] calcd for C₅₇H₈₃N₇O₇Si: 1006.62; found 1006.4.

### Intermediate 19. Synthesis of (2R)—N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-2-carboxamide

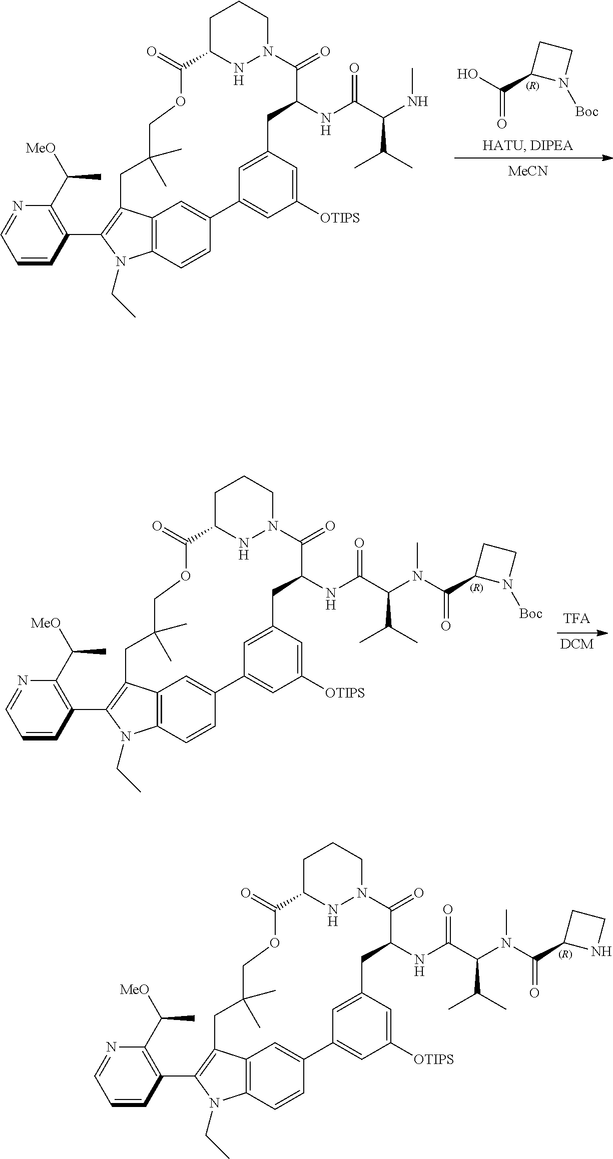

### Step 1: Synthesis of tert-butyl (2R)-2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)azetidine-1-carboxylate

To a mixture of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (1.0 g, 1.10 mmol), (R)-1-(tert-butoxycarbonyl)azetidine-2-carboxylic acid (0.33 g, 1.650 mmol) and HATU (1.25 g, 3.299 mmol) in MeCN (20 mL) at 0° C. was added DIPEA (0.94 mL, 5.499 mmol). The resulting mixture was stirred at 0° C. for 3 h and then was concentrated under reduced pressure. Purification by prep-TLC (10% MeOH/DCM) afforded the desired product (800 mg, 59.9% yield). LCMS (ESI) m/z: [M+H] calcd for C₆₁H₈₉N₇O₉Si: 1092.65; found 1092.6.

### Step 2: Synthesis of (2R)—N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-2-carboxamide

To a mixture of tert-butyl (2R)-2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)azetidine-1-carboxylate (400.0 mg, 0.366 mmol) in DCM (8.0 mL) at 0° C. was added TFA (4.0 mL). When the reaction was complete the mixture was concentrated under reduced pressure to afford the desired product (400 mg, crude). LCMS (ESI) m/z: [M+H] calcd for C₅₆H₈₁N₇O₇Si: 992.61; found 992.4.

### Intermediate 20. Synthesis of N-(sec-buty)-5-((6³S,4S)-1¹-ethyl-2⁵-hydroxy-10,10-dimethyl-4-((S)-3-methyl-2-(N-methyl-2-(methylamino)acetamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-N-methylnicotinamide

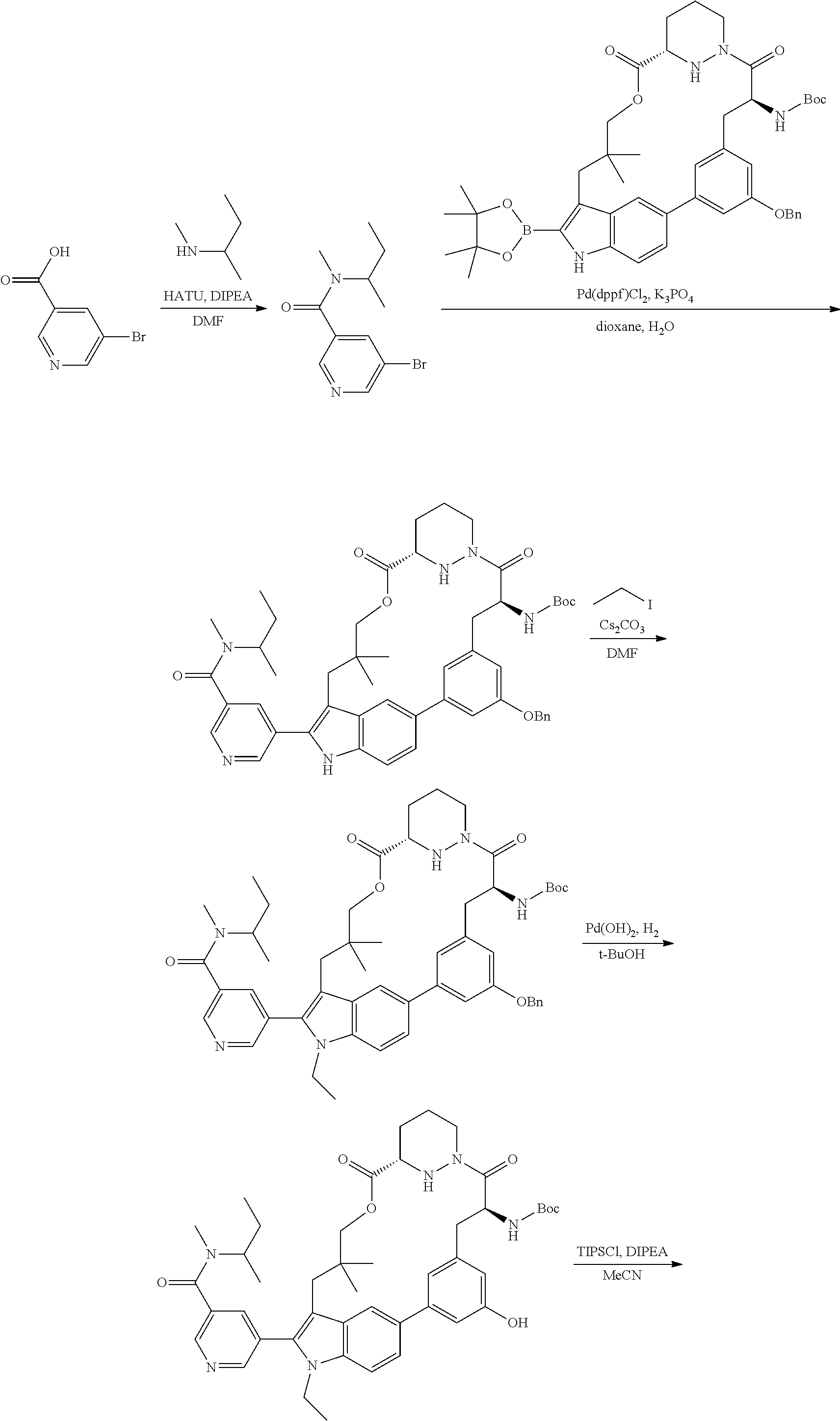

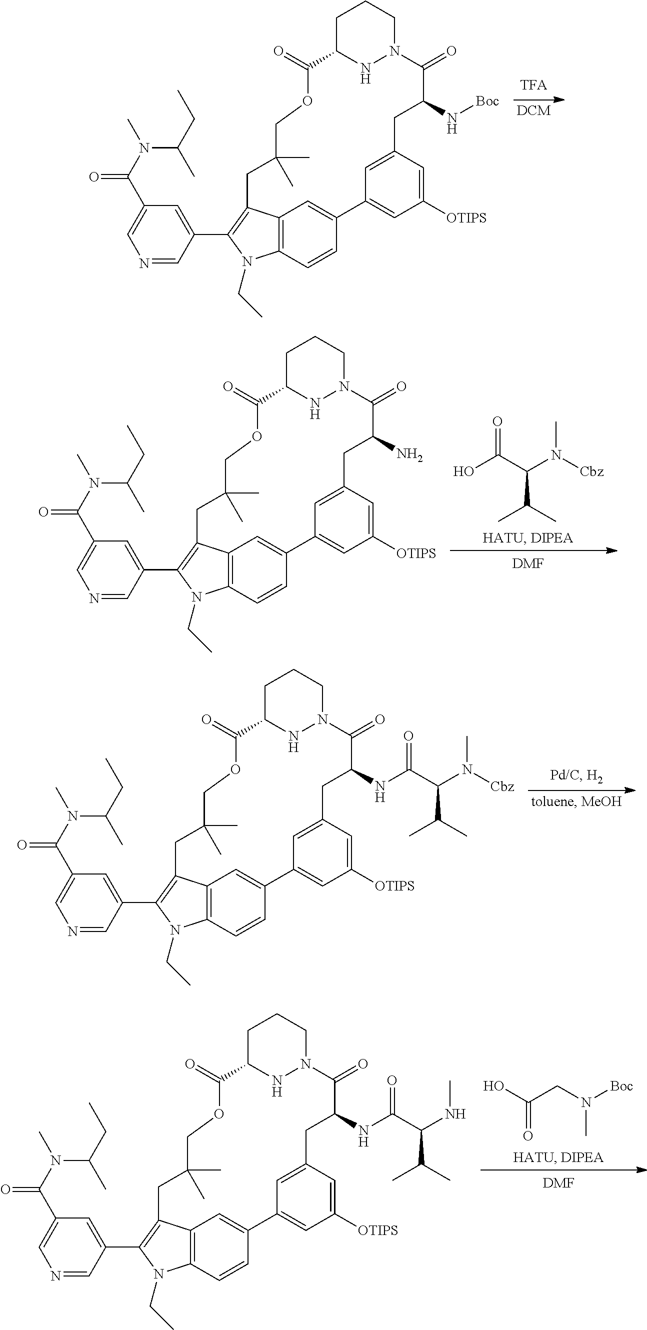

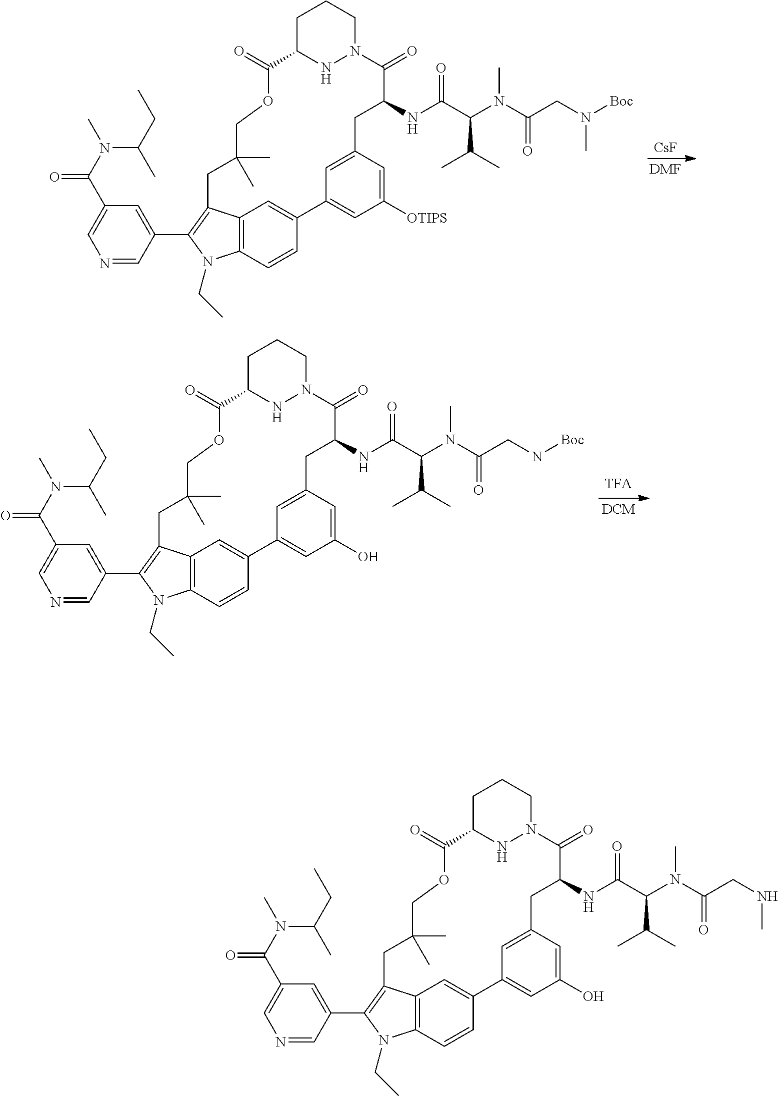

### Step 1: Synthesis of 5-bromo-N-(sec-butyl)-N-methylnicotinamide

To a solution of 5-bromonicotinic acid (2.0 g, 9.901 mmol) and HATU (5.65 g, 14,851 mmol) in DMF (40 mL) at 0° C. was added DIPEA (5.2 mL 9.9 mmol). The resulting mixture was stirred for 30 min at 0° C. and then N-methylbutan-2-amine (0.91 g, 10.396 mmol) was added. The resulting mixture was warmed to room temperature and stirred overnight, then diluted with H₂O (40 mL). The mixture was extracted with EtOAc (3×30 mL) and the combined organic layers were washed with brine (50 mL), dried over Na₂SO₄, filtered, and concentrated under reduced pressure. The residue was purified by silica gel chromatography (50% EtOAc/pet. ether) to afford the desired product (1.96 g, 73.2% yield), LCMS (ESI) m/z: [M+H] calcd for C₁₁H₁₅BrN₂O; 271.04; found 271.1.

### Step 2: Synthesis of tert-butyl ((6³S,4S)-25-(benzyloxy)-1²-(5-(sec-butyl(methyl)carbamoyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of 5-bromo-N-(sec-butyl)-N-methylnicotinamide (800.0 mg, 2.95 mmol) and K₃PO₃ (1.565 g, 7.376 mmol) in 1,4-dioxane (30.0 mL) and H₂O (6.0 mL) was added tort-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.81 g, 3.540 mmol) and Pd(dppf)Cl₂ (215.87 mg, 0.295 mmol). The resulting mixture was heated to 85° C. and stirred for 3 h. The mixture was then cooled to room temperature, quenched with H₂O, and extracted with EtOAc (3×100 mL). The combined organic layers were washed with H₂O (100 mL), dried over Na₂SO₄, filtered, and concentrated. The residue was purified by silica gel chromatography (10% MeOH/DCM) to afford the desired product (2.2 g, crude). LCMC (ESI) m/z: [M+H] calcd for C₅₀H₆₀N₆O₇: 857.46; found 857.5.

### Step 3: Synthesis of tert-butyl ((6³S,4S)-2⁵-(benzyloxy)-1²-(5-(sec-butyl(methyl)carbamoyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((6³S,4S)-25-(benzyloxy)-1²-(5-(sec-butyl(methyl)carbamoyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate (2.10 g, 2.450 mmol) and Cs₂CO₃ (2.39 g, 7.351 mmol) in DMF (20.0 mL) was added ethyl iodide (0.57 g, 3.675 mmol). The resulting mixture was stirred for 3 h at room temperature and was then quenched with H₂O (200 mL). The resulting mixture was extracted with EtOAc (3×100 mL) and the combined organic layers were washed with brine (50 mL), dried over Na₂SO₄, filtered, and concentrated under reduced pressure. The residue was purified by silica gel chromatography (10% MeOH/DCM) to afford the desired product (800 mg, 36.9% yield). LCMS (ESI) m/z: [M+H] calcd for C₅₂H₆₄N₆O₇: 885.49; found 885.5.

### Step 4: Synthesis of tert-butyl ((6³S,4S)-1²-(5-(sec-butyl(methyl)carbamoyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((6³S,4S)-2⁵-(benzyloxy)-1²-(5-(sec-butyl(methyl)carbamoyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (770.0 mg, 0.870 mmol) in tert-BuOH (20.0 mL) was added Pd(OH)₂/C (24.42 mg, 0.174 mmol). The resulting suspension was stirred overnight at 50° C. under a hydrogen atmosphere (1 atm). The mixture was then cooled to room temperature, filtered and the filter cake was washed with MeOH (3×30 mL). The filtrate was concentrated under reduced pressure to afford the desired product (810 mg, crude). LCMS (ESI) m/z: [M+H] calcd for C₄₅H₅₆N₆O₇: 795.44; found 795.5.

### Step 5: Synthesis of tert-butyl ((6³S,4S)-1²-(5-(sec-butyl(methyl)carbamoyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((6³S,4S)-1²-(5-(sec-butyl(methyl)carbamoyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (800.0 mg, 1.0 mmol) and DIPEA (0.876 mL, 5.031 mmol) in MeCN (10.0 mL) was added chlorotris(propan-2-yl)silane (291.02 mg, 1.509 mmol). The resulting mixture was stirred for 3 h and was then quenched with H₂O. The resulting mixture was extracted with EtOAc (3×50 mL) and the combined organic layers were washed with H₂O (3×30 mL), dried over Na₂SO₄, filtered, and concentrated under reduced pressure. The residue was purified by silica gel chromatography (10% MeOH/DCM) to afford the desired product (800 mg, 83.6% yield). LCMS (ESI) m/z: [M+H] calcd for C₅₄H₇₈N₆O₇Si: 951.58; found 950.8.

### Step 6: Synthesis of 5-((6³S,4S)-4-amino-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-N-(sec-butyl)-N-methylnicotinamide

To a solution of tert-butyl ((6³S,4S)-1²-(5-(sec-butyl(methyl)carbamoyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (720.0 mg, 0.757 mmol) in DCM (10.0 mL) at 0° C. was added TFA (3.0 mL, 40.4 mmol). The resulting mixture was stirred for 2 h and was then concentrated under reduced pressure. The residue was cooled to at 0° C. and neutralized with sat. aq. NaHCO₃. The resulting mixture was extracted with EtOAc (3×100 mL) and the combined organic layers were washed with H₂O (3×30 mL), dried over Na₂SO₄, filtered, and concentrated under reduced pressure to afford the desired product (540 mg, crude). LCMS (ESI) m/z: [M+H] calcd for C₄₉H₇₀N₆O₅Si: 851.53; found 851.8.

### Step 7: Synthesis of benzyl ((2S)-1-(((6³S,4S)-1²-(5-(sec-butyl(methyl)carbamoyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate

To a solution of 5-((6³S,4S)-4-amino-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-N-(sec-butyl)-N-methylnicotinamide (530.0 mg, 0.623 mmol) and N-((benzyloxy)carbonyl)-N-methyl-L-valine (198.23 mg, 0.747 mmol) in DMF (10.0 mL) were added HATU (473.49 mg, 1.245 mmol) and DIPEA (0.542 mL, 3.113 mmol). The resulting mixture was stirred for 2 h and was then quenched with H₂O and extracted with EtOAc (3×50 mL). The combined organic layers were washed with H₂O (3×30 mL), dried over Na₂SO₄, filtered, and concentrated under reduced pressure. The residue was purified by silica gel chromatography (10% MeOH/DCM) to afford the desired product (720 mg, crude). LCMS (ESI) m/z: [M+H] calcd for C₆₃H₈₇N₇O₈Si: 1098.65; found 1098.7.

### Step 8: Synthesis of N-(seco-butyl)-5-((6³S,4S)-1¹-ethyl-10,10-dimethyl-4-((S)-3-methyl-2-(methylamino)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-N-methylnicotinamide

To a solution of benzyl ((2S)-1-(((6³S,4S)-1²-(5-(sec-butyl(methyl)carbamoyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1H-8-oxa-1(5,3)-indola-6(1,3)-pyridazina-2(1,3)-benzenacycloundecaphane-4-yl)amino)-3-methyl-1-oxobutan-2-yl)(methyl)carbamate (670.0 mg, 0.610 mmol) in toluene (10.0 mL) and MeOH (1.0 mL) was added Pd/C (12.98 mg, 0.122 mmol). The suspension was stirred overnight under a hydrogen atmosphere (1 atm) and was then filtered, and the filter cake washed with MeOH (3×50 mL). The filtrate was concentrated under reduced pressure to afford the desired product (600 mg, crude). LCMS (ESI) m/z: [M+H] calcd for C₅₅H₈₁N₇O₆Si: 964.61; found 964.8.

### Step 9: Synthesis of tert-butyl (2-(((2S)-1-(((6³S,4S)-1²-(5-(sec-butyl(methyl)carbamoyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate

To a solution of N-(sec-butyl)-5-((6³S,4S)-1¹-ethyl-10,10-dimethyl-4-((S)-3-methyl-2-(methylamino)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-N-methylnicotinamide (490.0 mg, 0.508 mmol) and N-(tert-butoxycarbonyl)-N-methylglycine (114.4 mg, 0.610 mmol) in DMF (10.0 mL) was added HATU (386.39 mg, 1.016 mmol) and DIPEA (0.443 mL, 2.540 mmol). The resulting mixture was stirred for 2 h and was then quenched with H₂O and extracted with EtOAc (3×30 mL). The combined organic layers were washed with H₂O (3×30 mL), dried over Na₂SO₄, filtered, and concentrated under reduced pressure to afford the desired product (560 mg, 79.3% yield). LCMS (ESI) m/z: [M+H] calcd for C₆₃H₉₄N₈O₉Si: 1135.70; found 1136.3.

### Step 10: Synthesis of tert-butyl (2-(((2S)-1-(((6³S,4S)-1²-(5-(sec-butyl(methyl)carbamoyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate

To a solution of tert-butyl (2-(((2S)-1-(((6³S,4S)-1²-(5-(sec-butyl(methyl)carbamoyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamate (540.0 mg, 0.476 mmol) in DMF (10.0 mL) was added CsF (288.94 mg, 1.90 mmol). The resulting mixture was stirred for 2 h and was then quenched with H₂O and extracted with EtOAc (3×50 mL). The combined organic layers were washed with H₂O (3×30 mL), dried over Na₂SO₄, filtered, and concentrated under reduced pressure. The residue was purified by silica gel chromatography (10% MeOH/DCM) to afford the desired product (430 mg, crude). LCMS (ESI) m/z: [M+H] calcd for C₅₄H₇₄N₈O₉: 979.57; found 980.0.

### Step 11: Synthesis of N-(sec-butyl)-5-((6³S,4S)-1¹-ethyl-2⁵-hydroxy-10,10-dimethyl-4-((S)-3-methyl-2-(N-methyl-2-(methylamino)acetamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-N-methylnicotinamide

To a solution of tert-butyl (2-(((2S)-1-(((6³S,4S)-1²-(5-(sec-butyl(methyl)carbamoyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina- 2(1,3)-benzenacycloundecaphane-4-yl)amino)-3-methyl-1-oxobutan-2-yl)(methyl)amino)-2-oxoethyl)(methyl)carbamate (400.0 mg, 0.408 mmol) in DCM (10.0 mL) at 0° C. was added TFA (3.0 mL, 40.4 mmol). The reaction was stirred for 1 h and was the quenched with sat. aq. NaHCO₃. The mixture was extracted with EtOAc (3×50 mL) and the combined organic layers were washed with H₂O (3×30 mL), dried over Na₂SO₄, filtered, and concentrated under reduced pressure to afford the desired product (380 mg, crude). LCMS (ESI) m/z: [M+H] calcd for C₄₉H₆₆N₈O₇: 879.51; found 879.5.

### Intermediate 21. Synthesis of (2S)-2-(2-amino-N-methylacetamido)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

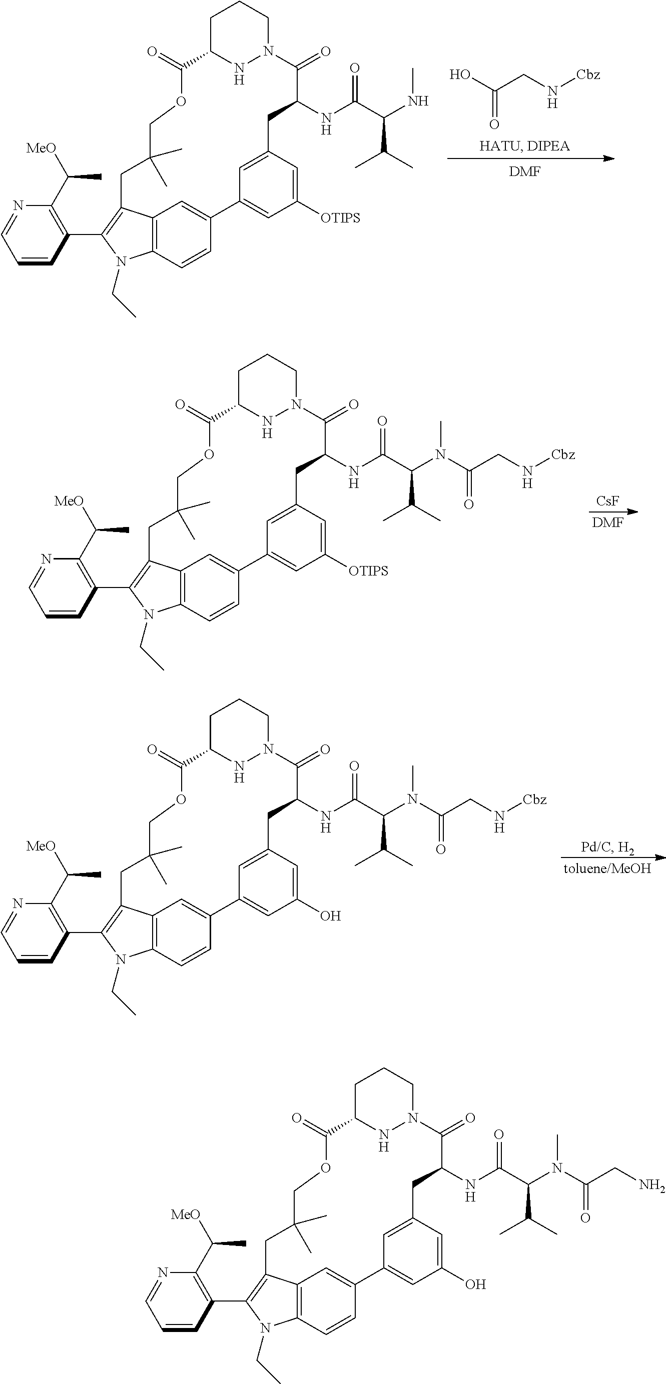

### Step 1: Synthesis of benzyl (2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)carbamate

To a solution of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (2.50 g, 2.75 mmol) and ((benzyloxy)carbonyl)glycine (690 mg, 3.30 mmol) in DMF (25 mL) at 0° C. was added HATU (2.10 g, 5.50 mmol) followed by DIPEA (1.5 mL, 8.25 mmol). The reaction mixture was stirred for 2 h and was then quenched with H₂O and extracted with EtOAc (3×50 mL). The combined organic layers were washed with H₂O (3×10 mL), dried with Na₂SO₄, filtered, and concentrated under reduced pressure. Purification by silica gel column chromatography (50% EtOAC/hexanes) afforded desired product (2.0 g, 72% yield). LCMS (ESI) m/z: [M+H] calcd for C₆₂H₈₅N₇O₉Si: 1100.63; found 1100.7.

### Step 2: Synthesis of benzyl (2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)carbamate

To a solution of benzyl (2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)carbamate (400 mg, 0.36 mmol) in DMF at 0° C. was added CsF (220 mg, 1.5 mmol). The reaction mixture was stirred for 2 h and was then quenched with H₂O and extracted with EtOAc (3×50 mL). The combined organic layers were washed with H₂O (3×10 mL), dried with Na₂SO₄, filtered, and concentrated under reduced pressure to afford the desired product (300 mg, 87% yield). LCMS (ESI) m/z: [M+H] calcd for C₅₃H₆₅N₇O₉: 944.49; found 944.4.

### Step 3: Synthesis of (2S)-2-(2-amino-N-methylacetamido)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of benzyl (2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)carbamate (300 mg, 0.32 mmol) in toluene (10 mL) and MeOH (1 mL) was added Pd/C (50 mg, 0.47 mmol). The suspension was stirred overnight under an atmosphere of hydrogen (1 atm). The reaction mixture was then was filtered and the filter cake was washed with EtOAc (3×10 mL). The filtrate was concentrated under reduced pressure to afford the desired product (180 mg, 43% yield). LCMS (ESI) m/z: [M+H] calcd for C₄₅H₅₉N₇O₇: 810.46; found 810.5.

### Intermediate 22. (3S,4R)—N-((2S)-1-(((6³S,4S)-1¹-ethyl-1²-(2-((S)-1-methoxyethyl)pyridin-3-yl)-10,10-dimethyl-5,7-dioxo-2-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N,4-dimethylpyrrolidine-3-carboxamide

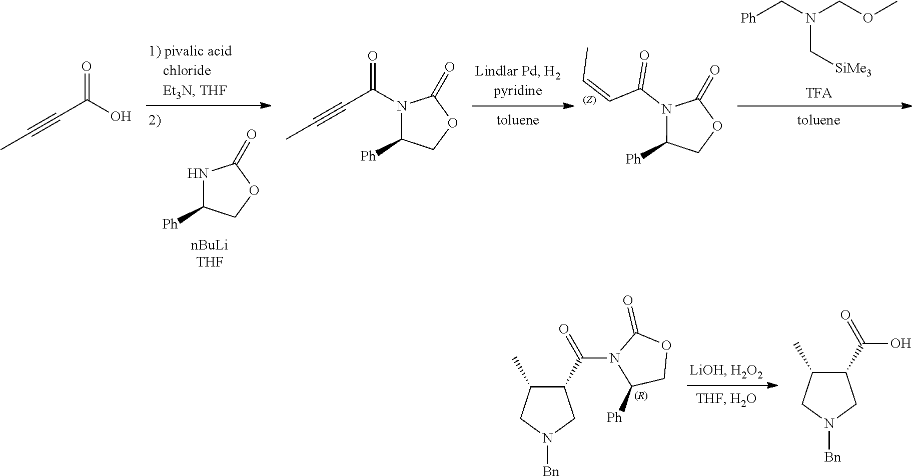

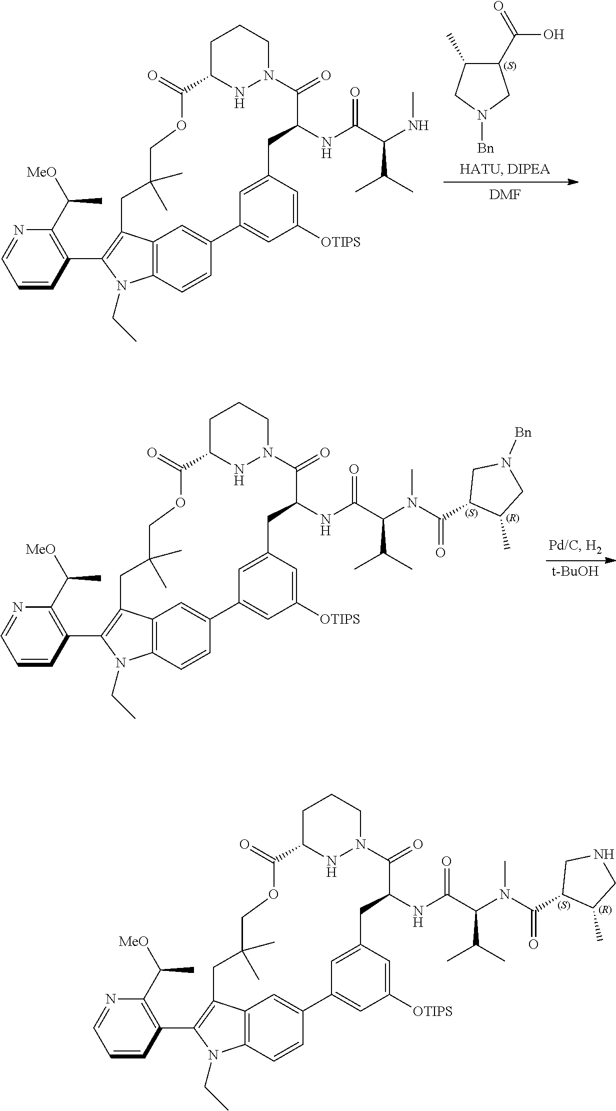

### Step 1: Synthesis of (R)-3-(but-2-ynoyl)-4-phenyloxazolidin-2-one

To a solution of 2-butynoic acid (5.0 g, 59.47 mmol) in THF (100 ml) at −78° C. was added pivalic acid chloride (7.39 g, 61.26 mmol) and Et₃N (6.2 mL, 61.85 mmol) and then the mixture was stirred for 15 min and then warmed to 0° C. and stirred for 45 min. In a second flask, to a solution of (4R)-4-phenyl-1,3-oxazolidin-2-one (9.70 g, 59.47 mmol) in THF (100 mL) at −78° C. was added n-BuLi (2.5 M in hexane, 25 mL, 62.5 mmol). The mixture was stirred at −78° C. for 15 min and was then added to the initial mixture. The combined solutions were warmed to room temperature and stirred overnight. The reaction solution was quenched with sat. NH₄Cl (200 ml) and then the mixture was extracted with EtOAc (3×100 mL. The combined organic layers were washed with brine (200 mL), dried over Na₂SO₄, filtered, and concentrated under reduced pressure. Purification by normal phase chromatography (20%/EtOAc/pet. ether) afforded the desired product (6.0 g, 44.0% yield). LCMS (ESI) m/z: [M+H] calcd for C₁₃H₁₁NO₃: 230.08; found 229.9.

### Step 2: Synthesis of (R,Z)-3-(but-2-enoyl)-4-phenyloxazolidin-2-one

To a solution of (R)-3-(but-2-ynoyl)-4-phenyloxazolidin-2-one (6.0 g, 26.17 mmol) in pyridine (6.0 mL) and toluene (60.0 mL) at 0° C. was added Lindlar Pd catalyst (594.57 mg, 2.88 mmol). The resulting mixture was stirred for 30 min at 0° C. under a hydrogen atmosphere (1 atm). The mixture was filtered, and the filter cake was washed with toluene (10.0 mL). The filtrate was concentrated under reduced pressure to afford the desired product (5.5 g, crude). LCMS (ESI) m/z: [M+H] calcd for C₁₃H₁₃NO₃: 232.10; found 231.9.

### Step 3: (R)-3-((3S,4R)-1-benzyl-4-methylpyrrolidine-3-carbonyl)-4-phenyloxazolidin-2-one

To a solution of (R,Z)-3-(but-2-enoyl)-4-phenyloxazolidin-2-one (3.0 g, 12.97 mmol) and benzyl(methoxymethyl)[(trimethylsilyl)methyl]amine (3.70 g, 15.57 mmol) in toluene (20.0 mL) at 0° C. was added TFA (1.30 mL, 0.87 mmol). The resulting mixture was warmed to room temperature and stirred overnight. The mixture was then concentrated under reduced pressure. The residue was purified by silica gel column chromatography (20% EtOAc/pet. ether) to afford the desired product (2 g, 42.3% yield). LCMS (ESI) m/z: [M+H] calcd for C₂₂H₂₄N₂O₃: 365.19; found 365.2.

### Step 4: Synthesis of (3S,4R)-1-benzyl-4-methylpyrrolidine-3-carboxylic acid

A solution of LiOH·H₂O (0.16 g, 6.860 mmol) and H₂O₂ (0.13 g, 3.76 mmol) in H₂O (5 mL) was added to a solution of (R)-3-((3S,4R)-1-benzyl-4-methylpyrrolidine-3-carbonyl)-4-phenyloxazolidin-2-one (1.0 g, 2.74 mmol) in THF (15.0 mL) at 0° C. The resulting mixture was stirred for 2 h and was then quenched with H₂O (30 mL) and sodium sulfite (0.69 g, 5.48 mmol) and the solution was extracted with EtOAc (2×50 mL). The aqueous phase was adjusted to pH 4 with NaH₂PO₄·H₂O and 10% HCl, and the brine was added. The solution was extracted with i-PrOH/DCM (1:3, 5×50 mL) and the combined organic layers were washed with brine (40 mL), dried over Na₂SO₄, filtered, and concentrated under reduced pressure to afford the desired product (400 mg, crude). LCMS (ESI) m/z. [M+H] calcd for C₁₃H₁₇NO₂: 220.14; found 220.2.

### Step 5: Synthesis of (3S,4R)-1-benzyl-N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N,4-dimethylpyrrolidine-3-carboxamide

To a mixture of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (414.67 mg, 0.456 mmol) and (3S,4R)-1-benzyl-4-methylpyrrolidine-3-carboxylic acid (200.0 mg, 0.912 mmol) in DMF (5.0 mL) at 0° C. was added HATU (693.58 mg, 1.824 mmol) and DIPEA (0.794 mL, 4.560 mmol). The resulting mixture was warmed to room temperature and stirred for 2 h. The reaction was quenched with the addition of sat. aq. NH₄Cl (40 mL) and then extracted with EtOAc (3×30 mL). The combined organic layers were washed with brine (2×20 mL), dried over Na₂SO₄, filtered, and concentrated under reduced pressure. The residue was purified by prep-TLC (9% MeOH/DCM) to afford the desired product (350 mg, 34.6% yield). LCMS (ESI) m/z: [M+H] calcd for C₆₅H₉₁N₇O₇Si: 1110.68; found 1110.9.

### Step 6: (3S,4R)—N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N,4-dimethylpyrrolidine-3-carboxamide

To a solution of (3S,4R)-1-benzyl-N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N,4-dimethylpyrrolidine-3-carboxamide (300.0 mg, 0.270 mmol) in t-BuOH (10.0 mL) was added Pd/C (60.08 mg, 0.565 mmol). The resulting suspension was stirred overnight under a hydrogen atmosphere (1 atm). The mixture was then filtered, the filter cake was washed with MeOH (2×5 mL), and the filtrate was concentrated under reduced pressure to afford the desired product (280 mg, crude). LCMS (ESI) m/z: [M+H] calcd for C₅₈H₈₅N₇O₇Si: 1020.64; found 1020.8.

### Intermediate 23. Synthesis of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)—N-methyl-2-(methylamino)propanamido)butanamide

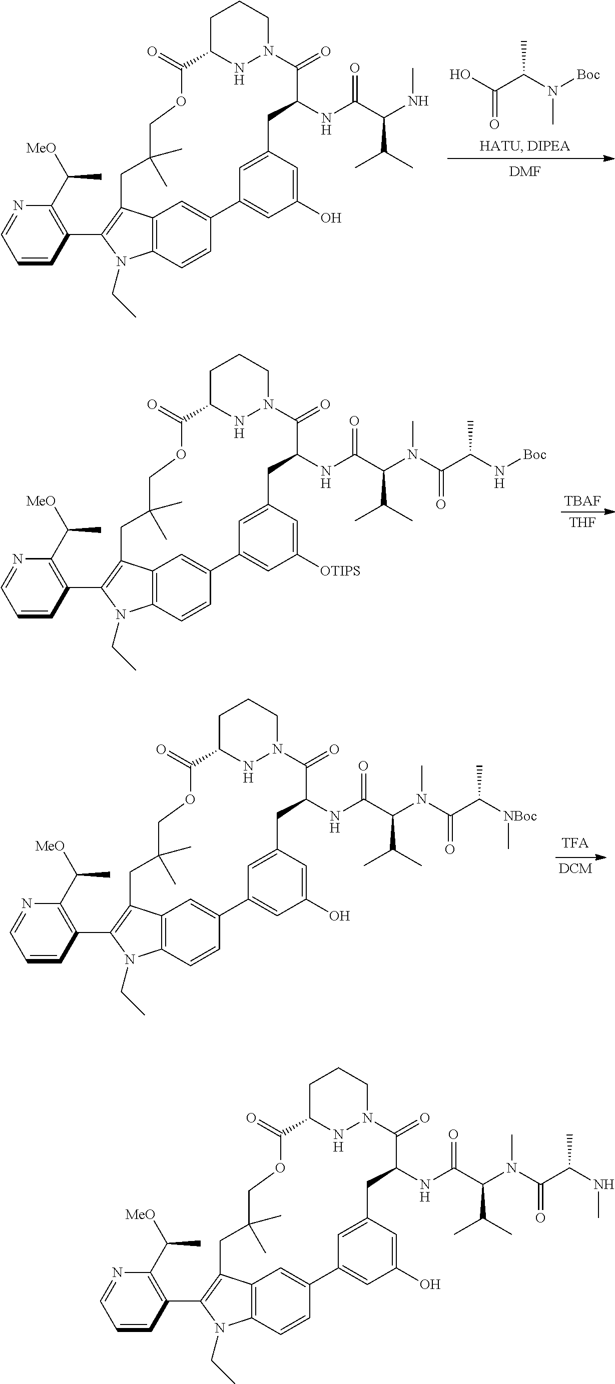

### Step 1: Synthesis of tert-butyl ((2S)-1-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-11H-8-oxa-1(5,3)-indola-6(1,3)-pyridazina-2(1,3)-benzenacycloundecaphane-4-yl)amino)-3-methyl-1-oxobutan-2-yl)(methyl)amino)-1-oxopropan-2-yl)(methyl)carbamate

To a solution of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (500.0 mg, 0.55 mmol), DIPEA (480 mL, 2.75 mmol) and (2S)-2-[(tert-butoxycarbonyl)(methyl)amino]propanoic acid (167.63 mg, 0.825 mmol) in DMF (5.0 mL) at 0° C. was added HATU (271.80 mg, 0.715 mmol). The mixture was warmed to room temperature and stirred for 4 h. The reaction was then quenched with H₂O and extracted with EtOAc (2×10 mL). The combined organic layers were washed with brine (5 mL), dried over Na₂SO₄, filtered, and concentrated under reduced pressure. The residue was purified by silica gel column chromatography (50% EtOAc/pet. ether) to afford the desired product (550 mg, 91.4% yield). LCMS (ESI) m/z: [M+H] calcd for C₆₁H₉₁N₇O₉Si: 1094.67; found 1094.5.

### Step 2: Synthesis of tert-butyl ((2S)-1-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-1-oxopropan-2-yl)(methyl)carbamate

To a solution of tert-butyl ((2S)-1-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-1-oxopropan-2-yl)(methyl)carbamate (540 mg, 0.493 mmol) in THF (5.0 mL) at 0° C. was added TBAF (1M in THF, 0.59 mL, 0.592 mmol). The mixture was warmed to room temperature and stirred for 30 min. The reaction was quenched with H₂O and was then extracted with EtOAc (2×10 mL). The combined organic layers were washed with brine (10 mL), dried over Na₂SO₄, filtered, After filtration, and concentrated under reduced pressure. The residue was purified by silica gel column chromatography (20% EtOAc/pet. ether) to afford the desired product (320 mg, 69.1% yield). LCMS (ESI) m/z: [M+H] calcd for C₅₂H₇₁N₇O₉: 938.534; found 938.4.

### Step 3: Synthesis of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-((S)—N-methyl-2-(methylamino)propanamido)butanamide

To a solution of tert-butyl ((2S)-1-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-1-oxopropan-2-yl)(methyl)carbamate (300.0 mg, 0.320 mmol) in DCM (3.0 mL) at 0° C. and was added TFA (1.0 mL). The mixture was warmed to room temperature and stirred for 2 h. The mixture was concentrated under reduced pressure to afford the desired product (300 mg, crude). LCMS (ESI) m/z: [M+H] calcd for C₄₇H₆₃N₇O₇: 838.49; found 838.4.

### Intermediate 24. Synthesis of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

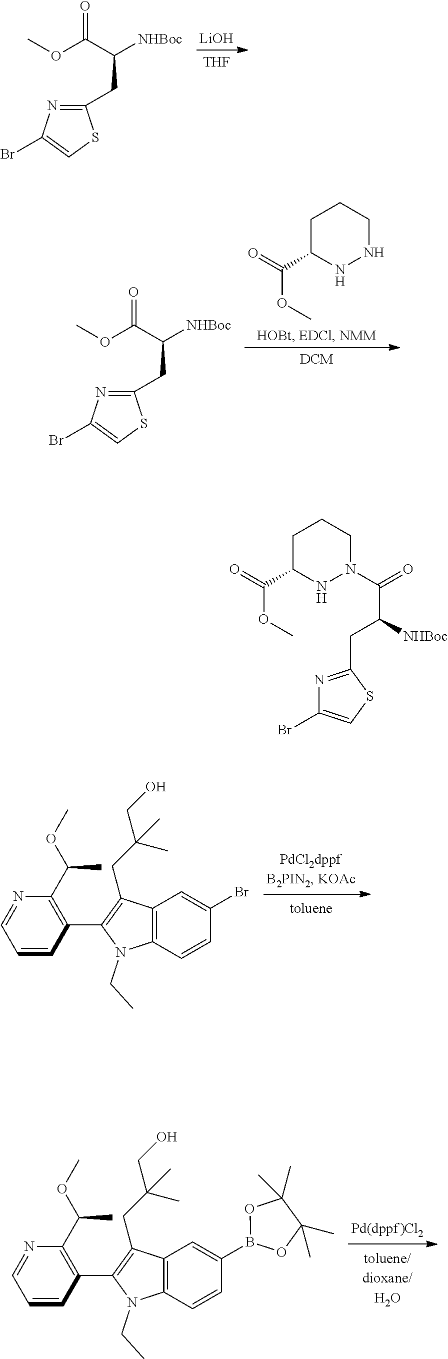

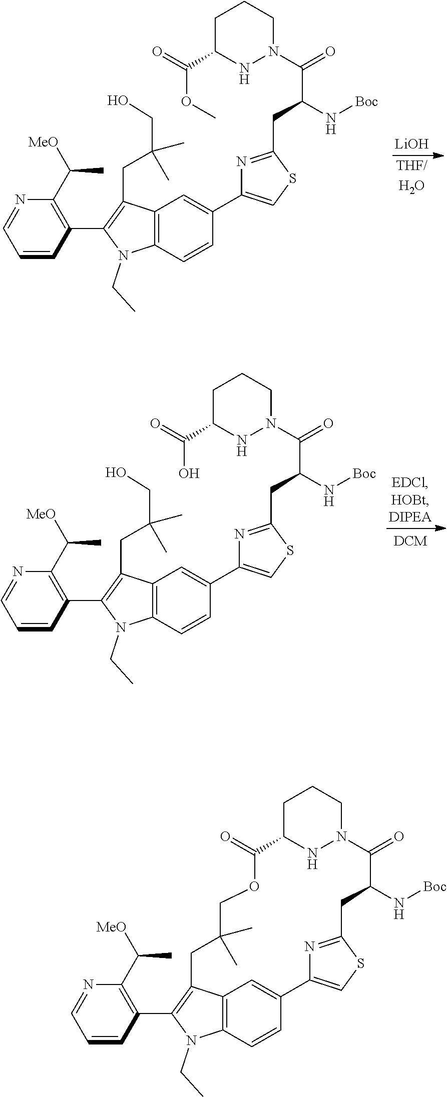

### Step 1: Synthesis of (S)-3-(4-bromothiazol-2-yl)-2-((tert-butoxycarbonyl)amino)propanoic acid

To a solution of methyl (2S)-3-(4-bromo-1,3-thiazol-2-yl)-2-[(tert-butoxycarbonyl)amino]propanoate (110 g, 301.2 mmol) in THF (500 mL) and H₂O (200 mL) at room temperature was added LiOH (21.64 g, 903.6 mmol). The resulting solution was stirred for 1 h and was then concentrated under reduced pressure. The resulting residue was adjusted to pH 6 with 1 M HCl and then extracted with DCM (3×500 mL). The combined organic layers were, dried over Na₂SO₄, filtered, and concentrated under reduced pressure to afford the desired product (108 g, crude). LCMS (ESI) m/z: [M+H] calcd for C₁₁H₁₅BrN₂O₄S: 351.00; found 351.0.

### Step 2: Synthesis of methyl (S)-1-((S)-3-(4-bromothiazol-2-yl)-2-((tert-butoxycarbonyl)amino)propanoyl)hexahydropyridazine-3-carboxylate

To a solution of (S)-3-(4-bromothiazol-2-yl)-2-((tert-butoxycarbonyl)amino)propanoic acid (70 g, 199.3 mmol) in DCM (500 mL) at 0° C. was added methyl (3S)-1,2-diazinane-3-carboxylate bis(trifluoroacetic acid) salt (111.28 g, 298.96 mmol), NMM (219.12 mL, 1993.0 mmol), EDCI (76.41 g, 398.6 mmol) and HOBt (5.39 g, 39.89 mmol). The resulting solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel chromatography (0→50% EtOAc/pet. ether) to afford the desired product (88.1 g, 92.6% yield). LCMS (ESI) m/z: [M+H] calcd for C₁₇H₂₅BrN₄O₅S: 477.08; found 477.1.

### Step 3: Synthesis of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol

To a solution of 3-(5-bromo-1-ethyl-2-(2-((S)-1-methoxyethyl)pyridin-3-yl)-1H-indol-3-yl)-2,2-dimethylpropan-1-ol (60 g, 134.7 mmol) in toluene (500 mL) at room temperature was added bis(pinacolato)diboron (51.31 g, 202.1 mmol), Pd(dppf)Cl₂ (9.86 g, 13.48 mmol) and KOAc (26.44 g, 269.4 mmol). Then reaction mixture was then heated to 90° C. and stirred for 2 h. The reaction solution was then cooled to room temperature and concentrated under reduced pressure. Purification by silica gel chromatography (0→50% EtOAc/pet. ether) afforded the desired product (60.6 g, 94.0% yield). LCMS (ESI) m/z: [M+H] calcd for C₂₉H₄₁BN₂O₄: 493.32; found 493.3.

### Step 4: Synthesis of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate

To a solution of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl (S)-1-((S)-3-(4-bromothiazol-2-yl)-2-((tert-butoxycarbonyl)amino)propanoyl)hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The resulting mixture was extracted with EtOAc (3×1000 mL) and the combined organic layers were dried over Na₂SO₄, filtered, and concentrated under reduced pressure. The residue was purified by silica gel chromatography (0→90% EtOAc/pet. ether) to afford the desired product (39.7 g, 85.4% yield). LCMS (ESI) m/z: [M+H] calcd for C₄₀H₅₄N₆O₇S: 763.39; found 763.3.

### Step 5: Synthesis of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid

To a solution of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate (39.7 g, 52.0 mmol) in THF (400 mL) and H₂O (100 mL) at room temperature was added LiOH·H₂O (3.74 g, 156.2 mmol). The resulting mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to afford the desired product (37.9 g, crude). LCMS (ESI) m/z: [M+H] calcd for C₃₉H₅₂N₆O₇S: 749.37; found 749.4.

### Step 6: Synthesis of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

To a solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCI (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel chromatography (0→70% EtOAc/pet. ether) to afford the desired product (30 g, 81.1% yield). LCMS (ESI) m/z: [M+H] calcd for C₃₉H₅₀N₆O₆S: 731.36; found 731.3.

### Intermediate 25. Synthesis of (6³S,4S)-4-amino-1¹-ethyl-2⁵-hydroxy-1²-(2-((S)-1-methoxyethyl)-5-(1-methylpiperidin-4-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

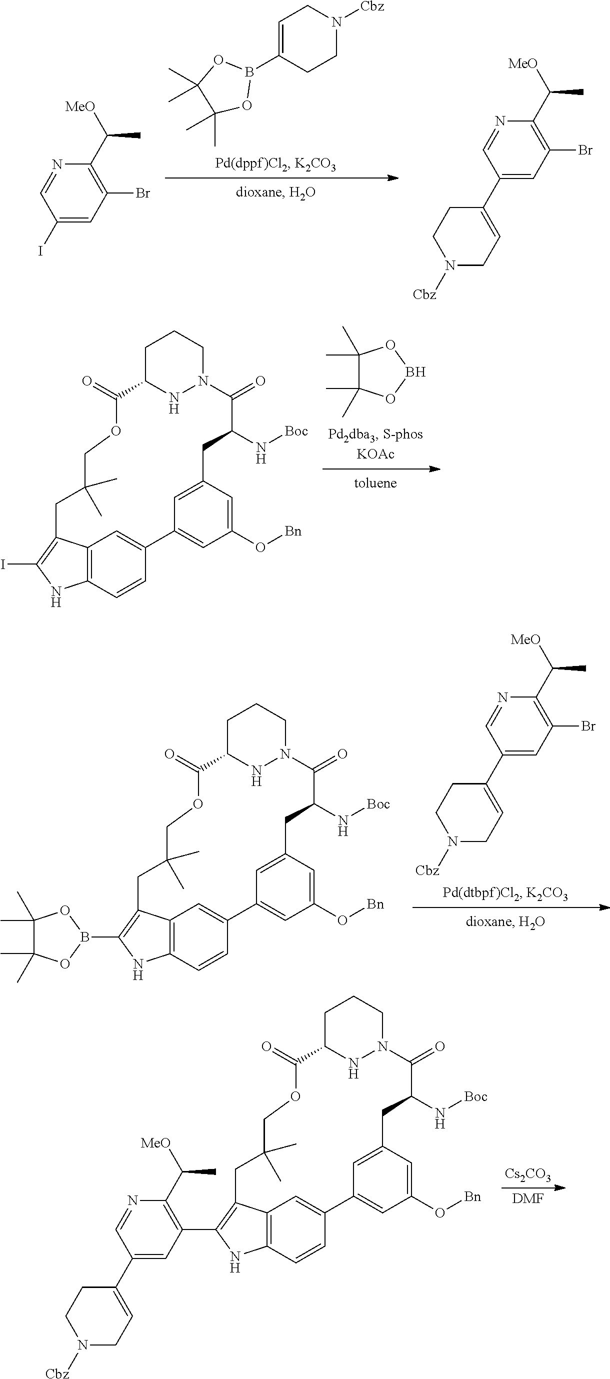

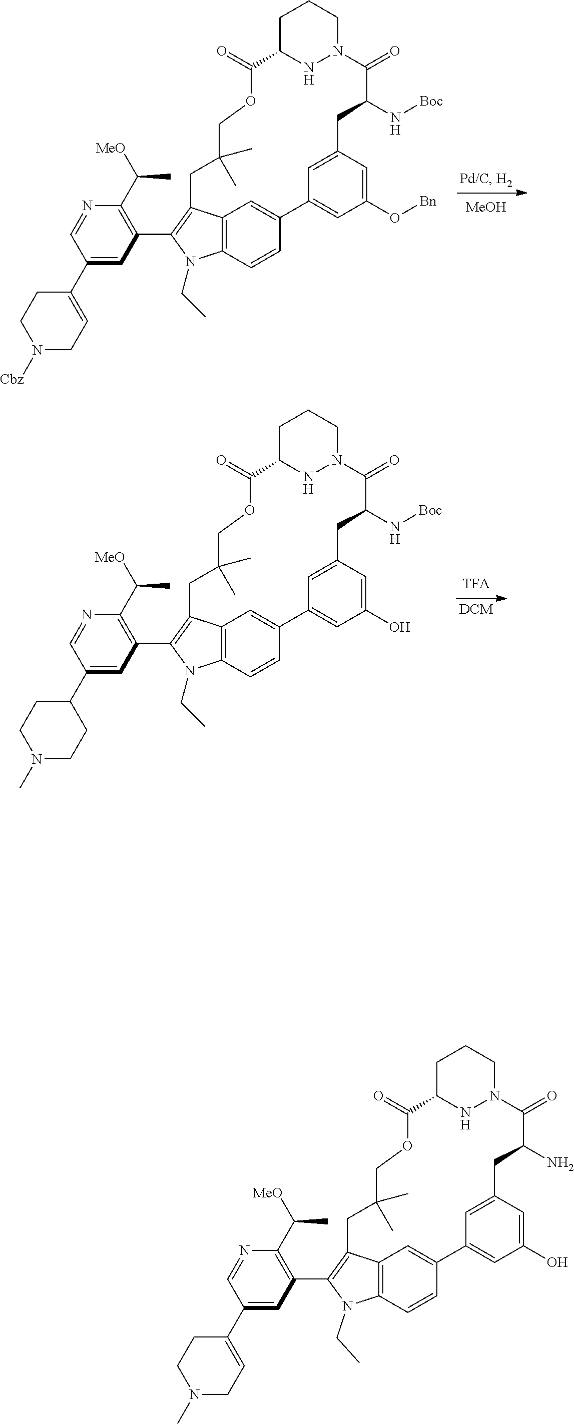

### Step 1: Synthesis of benzyl (S)-5-bromo-6-(1-methoxyethyl)-3′,6′-dihydro-[3,4′-bipyridine]-1′(2′H-carboxylate

To a solution of (S)-3-bromo-5-iodo-2-(1-methoxyethyl)pyridine (6.0 g, 17.55 mmol) and benzyl 4-(4,4,5,5-tetramethyl-1,3,2-dioxaborolan-2-yl)-3,6-dihydropyridine-1(2H)-carboxylate (7.23 g, 21.05 mmol) in dioxane (70 mL) and H₂O (14 mL) was added K₂CO₃ (6.06 g, 43.86 mmol) and Pd(dppf)Cl₂ (1.28 g, 1.76 mmol). The reaction mixture was heated to 60° C. and stirred for 3 h. The mixture was diluted with H₂O (50 mL) then extracted into EtOAc (3×100 mL). The combined organic layers were washed with brine (3×50 mL), dried over Na₂SO₄, filtered, and concentrated under reduced pressure. Purification by silica gel column chromatography (25% EtOAc/pet. ether) afforded the desired product (7.1 g, 94% yield). LCMS (ESI) m/z: [M+H] calcd for C₂₁H₂₃BrN₂O₃: 431.10; found 431.1.

### Step 2: Synthesis of tert-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((6³S,4S)-2⁵-(benzyloxy)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (5.0 g, 6.31 mmol), Pd₂(dba)₃ (690 mg, 757 μmol), S-Phos (0.78 g, 1.89 mmol), and KOAc (2.17 g, 22.08 mmol) in toluene (75 mL) was added 4,4,5,5-tetramethyl-1,3,2-dioxaborolane (5.65 g, 44.15 mmol). The reaction mixture was heated to 60° C. and stirred for 3 h. The reaction was quenched with H₂O at 0° C. then extracted into EtOAc (3×100 mL). The combined organic layers were washed with brine (3×30 mL), dried over Na₂SO₄, filtered, and concentrated under reduced pressure. Purification by silica gel column chromatography (50% EtOAc/pet. ether) afforded the desired product (4.5 g, 90% yield). LCMS (ESI) m/z: [M+H] calcd for C₇₅H₅₇BN₄O₈: 793.43; found 793.4.

### Step 3: Synthesis of benzyl 5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)-3′,6′-dihydro-[3,4′-bipyridine]-1′(2′H)-carboxylate

To a solution of tert-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (4.0 g, 5.05 mmol) and benzyl (S)-5-bromo-6-(1-methoxyethyl)-3′,6′-dihydro-[3,4′-bipyridine]-1′(2′H)-carboxylate (2.61 g, 6.06 mmol) in dioxane (50 mL) and H₂O (10 mL) was added K₂CO₃ (1.74 g, 12.6 mmol) and Pd(dtbpf)Cl₆ (330 mg, 505 μmol). The reaction mixture was heated to 70° C. After 3 h the reaction was diluted with H₂O (40 mL) and extracted into EtOAc (3×100 mL). The combined organic layers were washed with brine (2×50 mL), dried over Na₂SO₄, filtered, and concentrated under reduced pressure. Purification by silica gel column chromatography (50% EtOAc/pet. ether) afforded the desired product (4.1 g, 80% yield). LCMS (ESI) m/z: [M+H] calcd for C₆₀H₆₈N₆O₉: 1017.51; found 1017.4.

### Step 4: Synthesis of benzyl 5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-1²-yl)-6-((S)-1-methoxyethyl)-3′,6′-dihydro-[3,4′-bipyridine]-1′(2′H)-carboxylate

To a solution of benzyl 5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)-3′,6′-dihydro-[3,4′-bipyridine]-1′(2′H)-carboxylate (4.0 g, 3.93 mmol) and Cs₂CO₃ (3.84 g, 11.80 mmol) in DMF (30 mL) at 0° C. was added iodoethane (2.45 g, 15.73 mmol). The reaction mixture was warmed to room temperature. After 3 h the reaction mixture was diluted with H₂O (100 mL) and extracted into EtOAc (3×200 mL). The combined organic layers were washed with brine (3×100 mL), dried over Na₂SO₄, filtered, and concentrated under reduced pressure. Purification by silica gel column chromatography (66% EtOAc/pet. ether) afforded the desired product (1.4 g, 34% yield). LCMS (ESI) m/z: [M+H] calcd for C₆₂H₇₂N₆O₉: 1045.54; found 1045.5.

### Step 5: Synthesis of tert-butyl ((6³S,4S)-1¹-ethyl-2⁵-hydroxy-1²-(2-((S)-1-methoxyethyl)-5-(1-methylpiperidin-4-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

A solution of benzyl 5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)-3′,6′-dihydro-[3,4′-bipyridine]-1′(2′H)-carboxylate (1.29 g, 1.23 mmol) and Pd/C (700 mg) in MeOH (30 mL) was stirred for 72 h at room temperature under H₂ atmosphere. The reaction mixture was then filtered with MeOH (3×50 mL). The filtrate was concentrated under reduced pressure which afforded the desired product (850 mg, crude). LCMS (ESI) m/z: [M+H] calcd for C₄₈H₆₄N₆O₇: 837.49; found 837.7.

### Step 6: Synthesis of (6³S,4S)-4-amino-1¹-ethyl-2⁵-hydroxy-1²-(2-((S)-1-methoxyethyl)-5-(1-methylpiperidin-4-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-1¹-ethyl-2⁵-hydroxy-1²-(2-((S)-1-methoxyethyl)-5-(1-methylpiperidin-4-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (840 mg, 1.00 mmol) in DCM (10 mL) at 0° C. was added TFA (3.0 mL, 40.4 mmol). The reaction mixture was warmed to room temperature. After 2 h the reaction was cooled to 0° C., quenched with sat. at. NaHCO₃, and extracted into EtOAc (3×50 mL). The combined organic layers were washed with brine (2×30 mL), dried over Na₂SO₄, filtered, and concentrated under reduced pressure which afforded product (670 mg, crude). LCMS (ESI) m/z: [M+H] calcd for C₄₃H₅₆N₆O₅: 737.44; found 737.3.

### Intermediate 26. Synthesis of (6³S,4S)-4-amino-1¹-ethyl-2⁵-hydroxy-1²-(2-((S)-1-methoxyethyl)-5-(4-methylpiperazin-1-yl)pyridine-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

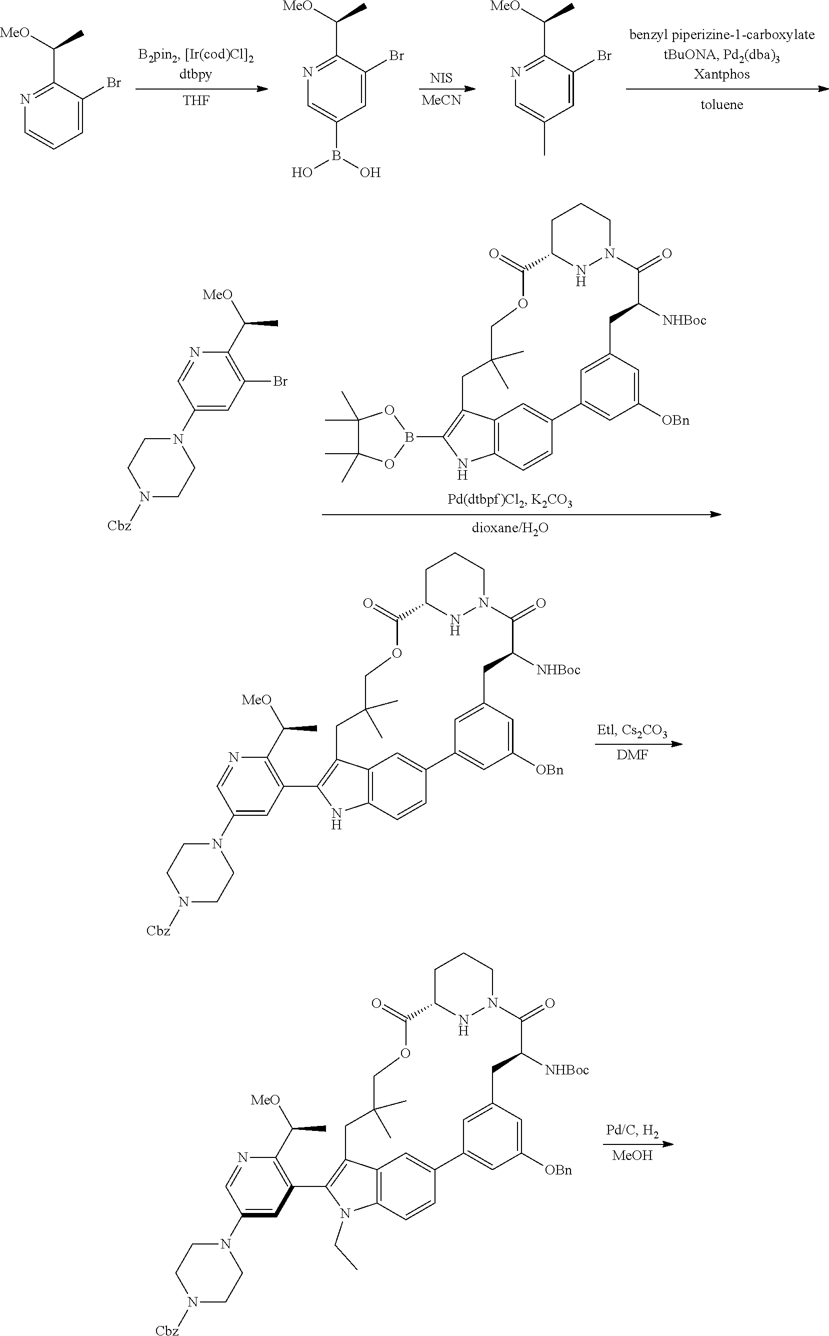

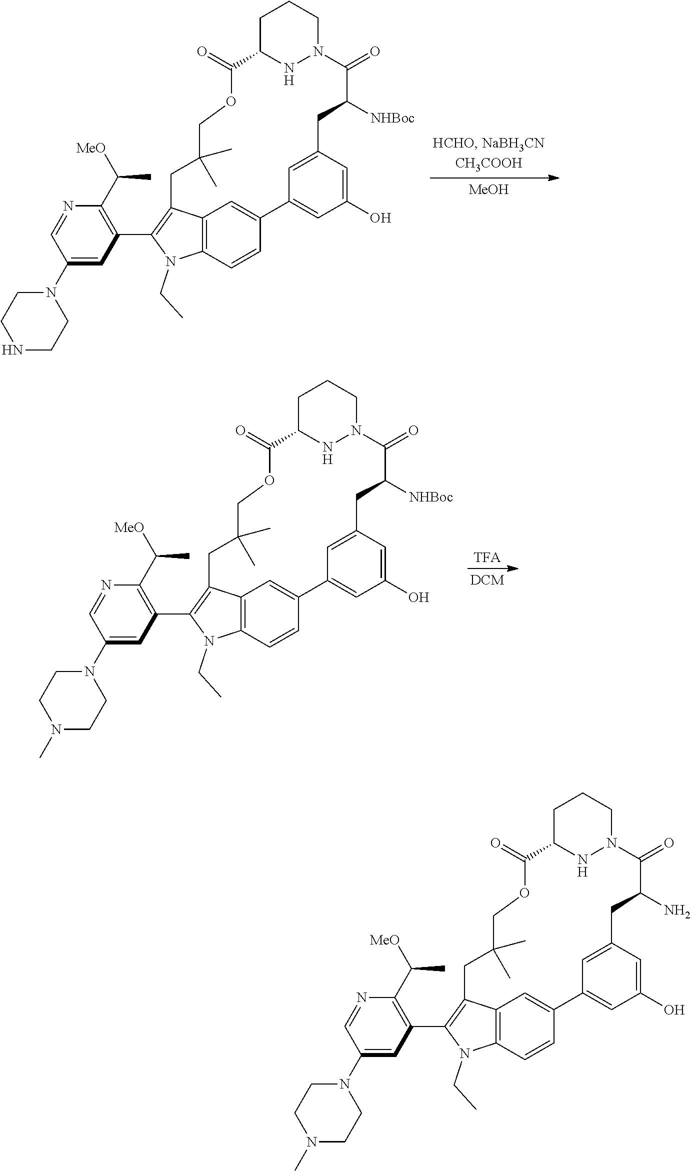

### Step 1: Synthesis of (S)-(5-bromo-6-(1-methoxyethyl)pyridin-3-yl)boronic acid

To a solution of (S)-3-bromo-2-(1-methoxyethyl)pyridine (40 g, 185 mmol) and bis(pinacolato)diboron (70.5 g, 278 mmol) in THF (1.6 L) at 75° C. was added 4,4′-di-tert-butyl-2,2′-bipyridine (7.45 g, 27.7 mmol) and [Ir(cod)Cl]₂ (1.24 mg, 1.85 mmol). After 16 h the mixture was concentrated under reduced pressure and the residue diluted with H₂O (1 L). The aqueous layer extracted with DCM/MeOH (2 L, 5:1), dried with Na₂SO₄, filtered, and concentrated under reduced pressure. Following purification by reverse phase chromatography (10→50% MeCN/H₂O, 0.1% HCO₂H) the combined product fractions were partially concentrated under reduced pressure. The aqueous layer was extracted with DCM/MeOH (3000 mL, 5:1), dried with Na₂SO₄, filtered, and concentrated under reduced pressure to afford the desired product (35.0 g, 65.5% yield). LCMS (ESI) m/z. [M+Na] calcd for C₈H₁₁BBrNO₃: 282.00; found 281.1.

### Step 2. Synthesis of (S)-3-bromo-5-iodo-2-(1-methoxyethyl)pyridine

To a solution of (S)-(5-bromo-6-(1-methoxyethyl)pyridin-3-yl)boronic acid (35.0 g, 135 mmol) in MeCN (100 mL) was added N-iodosuccinimide (60.6 g, 269 mmol). The resulting reaction mixture was stirred overnight and then concentrated under reduced pressure. Purification by normal phase chromatography (10% EtOAc/pet. ether) afforded the desired product (40.0 g, 78.1% yield). LCMS (ESI) m/z: [M+H] calcd for C₈H₉BrINO: 341.90; found 341.8.

### Step 3: Synthesis of benzyl (S)-4-(5-bromo-6-(1-methoxyethyl)pyridin-3-yl)piperazine-1-carboxylate

To a solution of (S)-3-bromo-5-iodo-2-(1-methoxyethyl)pyridine (7.0 g, 20.5 mmol) and benzyl piperazine-1-carboxylate (9.0 g, 40.8 mmol) in toluene (70 mL) were added Pd₂(dba)₃ (375 mg, 0.409 mmol), Xantphos (1.18 g, 2.05 mmol) and sodium tert-butoxide (2.29 g, 24.6 mmol). The resulting mixture was heated to 120° C. and stirred for 16 h then cooled to room temperature and concentrated under reduced pressure. Purification by normal phase chromatography (25% EtOAc/pet. ether) afforded the desired product (5.0 g, 50.6% yield). LCMS (ESI) m/z: [M+H] calcd for C₂₀H₂₄BrN₃O₃: 434.11; found 434.0.

### Step 4: Synthesis of benzyl 4-(5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate

To a solution of benzyl (S)-4-(5-bromo-6-(1-methoxyethyl)pyridin-3-yl)piperazine-1-carboxylate (3.29 g, 7.56 mmol) and tert-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (50 g, 6.31 mmol) dioxane (40 mL) and H₂O (10 mL) were added K₂CO₃ (1.74 g, 12.614 mmol) and Pd(dtbpf)Cl₂ (822 mg, 1.26 mmol) and the resulting mixture was heated to 80° C. for 2 h. The reaction mixture was then concentrated under reduced pressure and diluted with H₂O (1 L). The aqueous layer was extracted with EtOAc (3×200 mL) and the combined organic layers were washed with H₂O, dried with Na₂SO₄, filtered, and concentrated under reduced pressure. Purification by normal phase chromatography (50% EtOAc/pet. ether) afforded the desired product (5.0 g, 73.8% yield). LCMS (ESI) m/z: [M+H] calcd for C₅₉H₆₉N₇O₉: 1020.54; found 1020.6.

### Step 5: Synthesis of benzyl 4-(5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate

To a stirred solution of benzyl 4-(5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-1²-yl)-6-((S)-1-methoxyethyl)pyridin-3-yl)piperazine-1-carboxylate (5.0 g, 5 mmol) in DMF (50 mL) at 0° C. was added Cs₂CO₃ (3.19 g, 9.80 mmol) and ethyl iodide (1.53 g, 10 mmol). The resulting mixture was stirred for 2 h at room temperature and then diluted with H₂O (200 mL). The aqueous layer was extracted with EtOAc (3×100 mL) and the combined organic layers were washed with H₂O, dried with Na₂SO₄, filtered, and concentrated under reduced pressure. Purification by normal phase chromatography (33% EtOAc/pet. ether) afforded the desired product (1.8 g, 35% yield). LCMS (ESI) m/z: [M+H] calcd for C₆₁H₇₃N₇O₉: 1048.56; found 1048.4.

### Step 6: Synthesis of tert-butyl ((6³S,4S)-1¹-ethyl-2⁵-hydroxy-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred solution of benzyl 4-(5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (1.80 g, 1.72 mmol) in MeOH (20 mL) was added Pd/C (900 mg). The resulting mixture was stirred for 2 h at room temperature under a hydrogen atmosphere, filtered, and the filter cake washed with MeOH. The filtrate was concentrated under reduced pressure to afford the crude desired product which was used without further purification. LCMS (ESI) m/z: [M+H] calcd for C₄₆H₆₁N₇O₇: 824.47; found 824.3.

### Step 7: Synthesis of tert-butyl((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred solution of tert-butyl ((6³S,4S)-1¹-ethyl-2⁵-hydroxy-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate carbamate (590 mg, 0.716 mmol) and HCHO (129 mg, 1.43 mmol, 37 wt % in H₂O) in MeOH (6 ml) at 0° C. were added CH₃COOH (122 mg, 2.02 mmol) and NaBH₃CN (85.3 mg, 1.35 mmol). The resulting mixture was warmed to room temperature and stirred for 2 h. The reaction mixture was then concentrated under reduced pressure and diluted with H₂O (100 mL). The aqueous layer was extracted with EtOAc (3×100 mL) and the combined organic layers were washed with H₂O, dried with Na₂SO₄, filtered, concentrated under reduced pressure. pressure to afford the crude desired product which was used without further purification. LCMS (ESI) m/z: [M+H] calcd for C₄₇H₆₃N₇O₉: 838.49; found 838.4.

### Step 8: Synthesis of (6³S,4S)-4-amino-1¹-ethyl-2⁵-hydroxy-1²-(2-((S)-1-methoxyethyl)-5-(4-methylpiperazin-1-yl)pyridine-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred solution of tert-butyl((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (590 mg, 0.704 mmol) in DCM (6 mL) at 0° C. was added TFA (3.0 mL). The resulting mixture was stirred for 30 min and then concentrated under reduced pressure to afford the crude desired product which was used without further purification. LCMS (ESI) m/z: [M+H] calcd for C₄₂H₅₅N₇O₅: 738.44; found 738.4.

### Intermediate 27. Synthesis of (6³S,4S,Z)-4-amino-1¹-ethyl-1²-(2-((S)-1-methoxyethyl)-5-(4-methylpiperazin-1-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

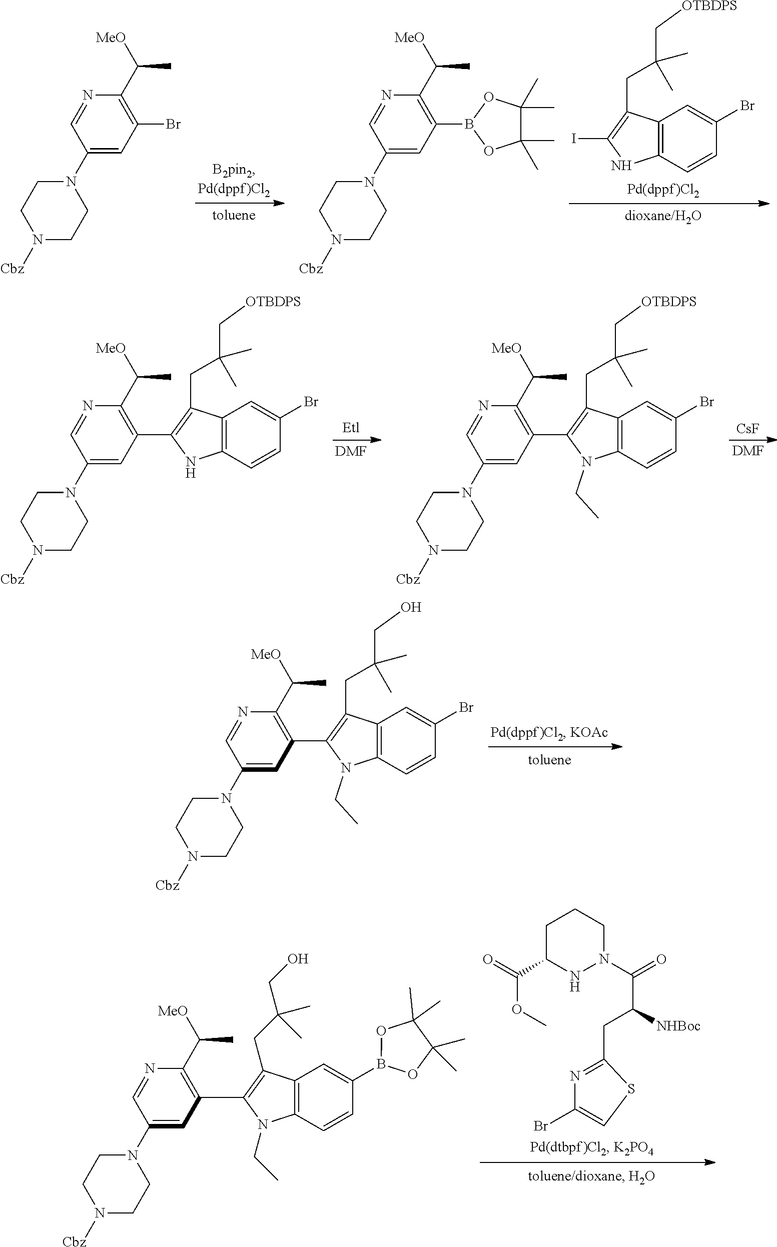

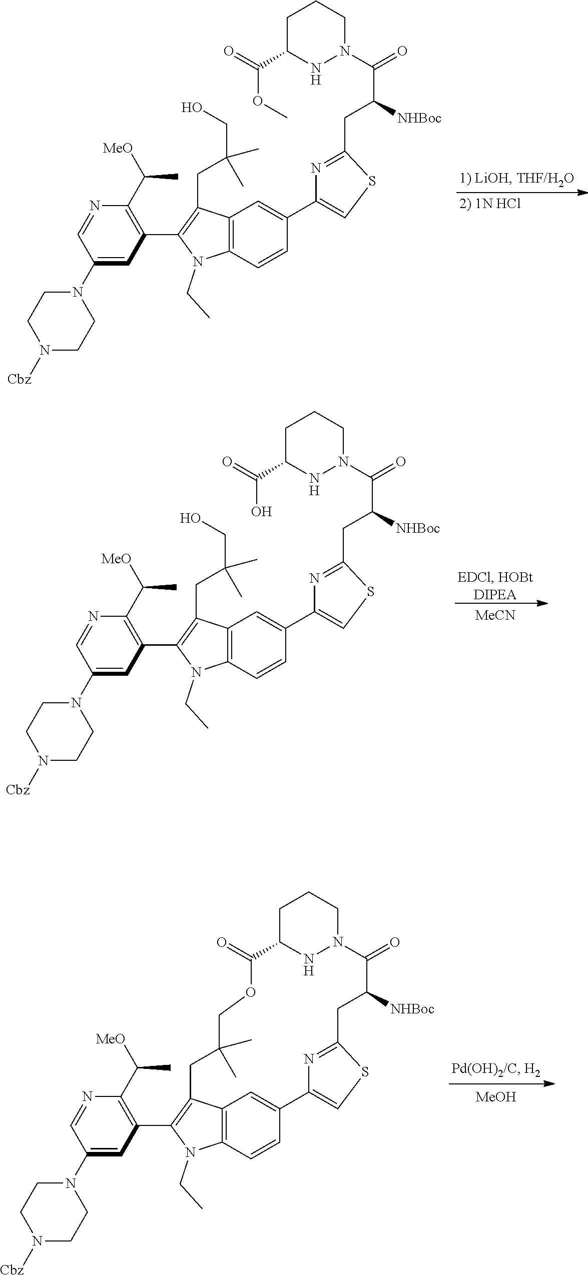

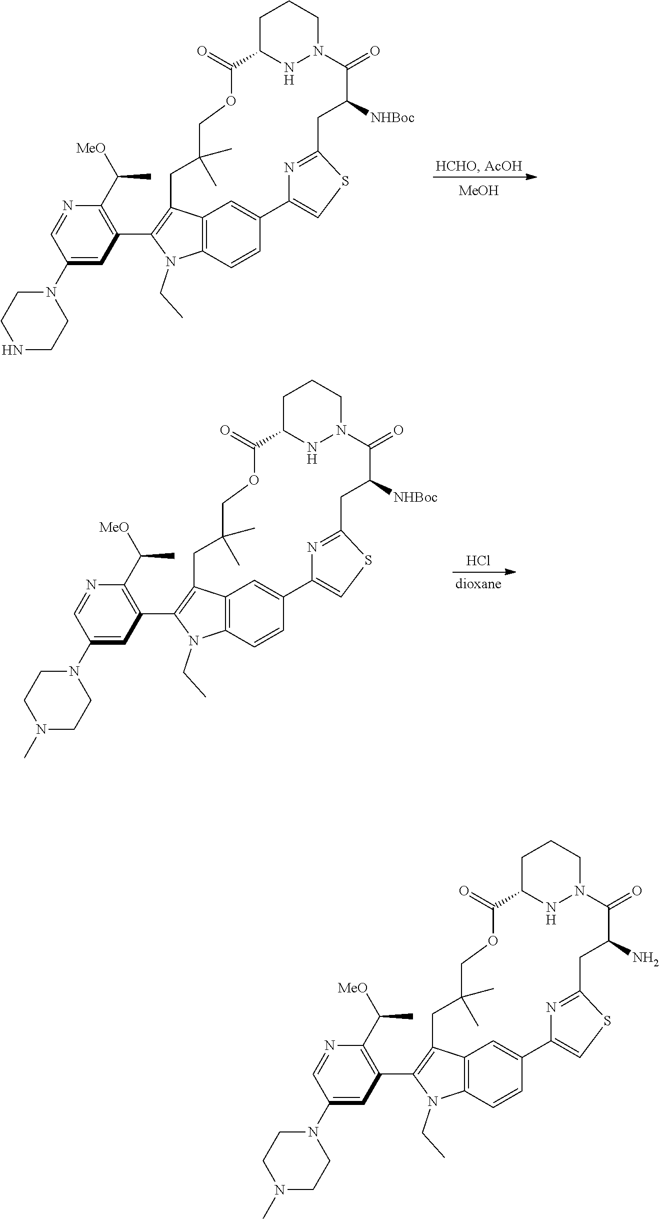

### Step 1: Synthesis of benzyl (S)-4-(5-bromo-6-(1-methoxyethyl)pyridin-3-yl)piperazine-1-carboxylate

Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.821 mmol), bis(pinacolato)diboron (86.82 g, 341.903 mmol), Pd(dppf)Cl₂ (22.74 g, 31.082 mmol), KOAc (76.26 g, 777.052 mmol), and toluene (1 L). The resulting solution was stirred for 2 days at 90° C. in an oil bath. The reaction mixture was cooled to room temperature. The resulting mixture was concentrated under reduced pressure. The residue was purified by neutral alumina column chromatography (25% EtOAc/hexanes) to afford the desired product (167 g, crude). LCMS (ESI) m/z: [M+H] calcd for C₂₆H₃₆BN₃O₅: 481.3; found 482.1.

### Step 2: Synthesis of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate

Into a 3-L 3-necked round-bottom flask purged and maintained with an inert atmosphere of argon, was placed (S)-4-(6-(1-methoxyethyl)-5-(4,4,5,5-tetramethyl-1,3,2-dioxaborolan-2-yl)pyridin-3-yl)piperazine-1-carboxylate (167 g, 346.905 mmol), 5-bromo-3-[3-[(tert-butyldiphenylsilyl)oxy]-2,2-dimethylpropyl]-2-iodo-1H-indole (224.27 g, 346.905 mmol), Pd(dppf)Cl₂ (25.38 g, 34.69 mmol), dioxane (600 mL), H₂O (200 mL), K₃PO₄ (184.09 g, 867.262 mmol), and toluene (200 mL). The resulting solution was stirred for overnight at 70° C. in an oil bath. The reaction mixture was cooled to room temperature after reaction completed. The resulting mixture was concentrated under reduced pressure. The residue was purified by normal phase column chromatography (50% EtOAc/hexanes) to afford the desired product (146 g, 48.2% yield). LCMS (ESI) m/z: [M+H] calcd for C₄₉H₅₇BrN₄O₄Si: 872.3; found 873.3.

### Step 3: Synthesis of benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate

To a stirred mixture of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (146 g, 167.047 mmol) and Cs₂CO₃ (163.28 g, 501.14 mmol) in DMF (1200 mL) was added C₂H₅I (52.11 g, 334.093 mmol) in portions at 0° C. under N₂ atmosphere. The final reaction mixture was stirred at room temperature for 12 h. The resulting mixture was diluted with EtOAc (1 L) and washed with brine (3×1.5 L). The organic layers were dried over anhydrous Na₂SO₄. After filtration, the filtrate was concentrated under reduced pressure to afford the desired product (143 g, crude). LCMS (ESI) m/z: [M+H] calcd for C₅₁H₆₁BrN₄O₄Si: 900.4; found 901.4.

### Step 4: Synthesis of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate

To a stirred mixture of benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate (143 g, 158.526 mmol) in DMF (1250 mL) was added CsF (72.24 g, 475.578 mmol). The reaction mixture was stirred at 60° C. for 2 days under N₂ atmosphere. The resulting mixture was diluted with EtOAc (1 L) and washed with brine (3×1 L). The organic phase was concentrated under reduced pressure and the residue was purified by silica gel column chromatography to afford two atropisomers of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate A (38 g, 36% yield, RT=1.677 min in 3 min LCMS (0.1% FA)) and B (34 g, 34% yield, RT=1.578 min in 3 min LCMS (0.1% FA)). LCMS (ESI) m/z: [M+H] calcd for C₃₅H₄₃BrN₄O₄: 663.2; found 662.2.

### Step 5: Synthesis of benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate

Into a 500-mL 3-necked round-bottom flask purged and maintained with an inert atmosphere of nitrogen, was placed benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate A (14 g, 21.095 mmol), bis(pinacolato)diboron (5.89 g, 23.205 mmol), Pd(dppf)Cl₂ (1.54 g, 2.11 mmol), KOAc (5.18 g, 52.738 mmol), and toluene (150 mL). The resulting solution was stirred for 5 h at 90° C. in an oil bath. The reaction mixture was then cooled to room temperature. The resulting mixture was concentrated under reduced pressure and the residue was purified by silica gel column chromatography to afford the desired product (12 g, 76.0% yield). LCMS (ESI) m/z: [M+H] calcd for C₄₁H₅₅BN₄O₆: 710.4; found 711.3.

### Step 6: Synthesis of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate

Into a 250-mL round-bottom flask purged and maintained with an inert atmosphere of argon, was placed benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (10.8 g, 15.196 mmol), methyl (3S)-1-[(2S)-3-(4-bromo-1,3-thiazol-2-yl)-2-[(tert-butoxycarbonyl)amino]propanoyl]-1,2-diazinane-3-carboxylate (7.98 g, 16.716 mmol), Pd(dtbpf)Cl₂ (0.99 g, 1.52 mmol), K₃PO₄ (8.06 g, 37.99 mmol), toluene (60 mL), dioxane (20 mL), and H₂O (20 mL). The resulting solution was stirred for 3 h at 70° C. in an oil bath. The reaction mixture was then cooled to room temperature. The resulting solution was extracted with EtOAc (2×50 mL) and concentrated under reduced pressure. The residue was purified by normal phase column chromatography to afford the desired product (8 g, 50.9% yield). LCMS (ESI) m/z: [M+H] calcd for C₅₂H₆₈N₈O₉S: 980.5; found 980.9.

### Step 7: Synthesis of (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid

To a stirred mixture of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (12 g, 12.230 mmol) in THF (100 mL) and H₂O (100 mL) was added LiOH (2.45 g, 61.148 mmol) under N₂ atmosphere and the resulting mixture was stirred for 2 h at room temperature. The resulting mixture was concentrated under reduced pressure and the pH of aqueous phase was acidified to 5 with HCl (1N) at 0° C. The aqueous layer was extracted with DCM (3×100 mL). The organic phase was concentrated under reduced pressure to afford the desired product (10 g, 84.5% yield). LCMS (ESI) m/z: [M+H] calcd for C₅₁H₆₆N₈O₉S: 966.5; found 967.0.

### Step 8: Synthesis of benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate

Into a 3-L round-bottom flask purged and maintained with an inert atmosphere of nitrogen, was placed (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (18 g, 18.61 mmol), MeCN (1.8 L), DIPEA (96.21 g, 744.417 mmol), EDCI (107.03 g, 558.313 mmol), HOBT (25.15 g, 186.104 mmol). The resulting solution was stirred for overnight at room temperature. The resulting mixture was concentrated under reduced pressure after reaction completed. The resulting solution was diluted with DCM (1 L) and was washed with HCl (3×1 L, 1N aqueous). The resulting mixture was washed with H₂O (3×1 L) and then the organic layer was concentrated. The residue was purified by normal phase column chromatography (50% EtOAc/hexanes) to afford the desired product (10.4 g, 54.9% yield). LCMS (ESI) m/z: [M+H] calcd for C₅₁H₆₄N₈O₈S: 948.5; found 949.3.

### Step 9: Synthesis of tert-butyl ((6³S,4S,Z)-11-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

Into a 250-mL round-bottom flask purged and maintained with an inert atmosphere of nitrogen, was placed benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate (10.40 g, 10.957 mmol), Pd(OH)₂/C (5 g, 46.984 mmol), and MeOH (100 mL). The resulting solution was stirred for 3 h at room temperature under a 2 atm H₂ atmosphere. The solids were filtered out and the filter cake was washed with MeOH (3×100 mL). The combined organic phase was concentrated under reduced pressure to afford the desired product (8.5 g, 90.4% yield). LCMS (ESI) m/z: [M+H] calcd for C₄₃H₅₈N₈O₆S: 814.4; found 815.3.

### Step 10: Synthesis of tert-butyl ((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

Into a 1000-mL round-bottom flask purged and maintained with an inert atmosphere of nitrogen, was placed tert-butyl ((6³S,4S,Z)-11-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.5 g, 10.429 mmol), MeOH (100 mL), AcOH (1.88 g, 31.286 mmol) and stirred for 15 min. Then HCHO (1.88 g, 23.15 mmol, 37% aqueous solution) and NaBH₃CN (788 mg, 12.5 mmol) was added at room temperature. The resulting solution was stirred for 3 hr. The resulting mixture was quenched with H₂O (100 mL) and concentrated under reduced pressure to remove MeOH. The resulting solution was diluted with DCM (300 mL) and was washed with H₂O (3×100 mL). The organic phase was concentrated under reduced pressure to afford the desired product (8.2 g, 90.1% yield). LCMS (ESI) m/z: [M+H] calcd for C₄₄H₆₀N₈O₆S: 828.4; found 829.3.

### Step 10: Synthesis of (6³S,4S,Z)-4-amino-1¹-ethyl-1²-(2-((S)-1-methoxyethyl)-5-(4-methylpiperazin-1-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

Into a 250-mL round-bottom flask purged and maintained with an inert atmosphere of nitrogen, was placed tert-butyl ((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate (8.20 g, 9.891 mmol) and dioxane (40 mL), followed by the addition of HCl in 1,4-dioxane (4M, 40 mL) at 0° C. The resulting solution was stirred for 1 h at 0° C. The mixture was then concentrated under reduced pressure. The resulting solution was diluted with DCM (600 mL) and sat. aq. NaHCO₃ (400 mL). The organic phase was then washed twice with brine (500 mL). The organic phase was concentrated under reduced pressure to afford the desired product (7.2 g, 94.9% yield).

### Intermediate 28. Synthesis of (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

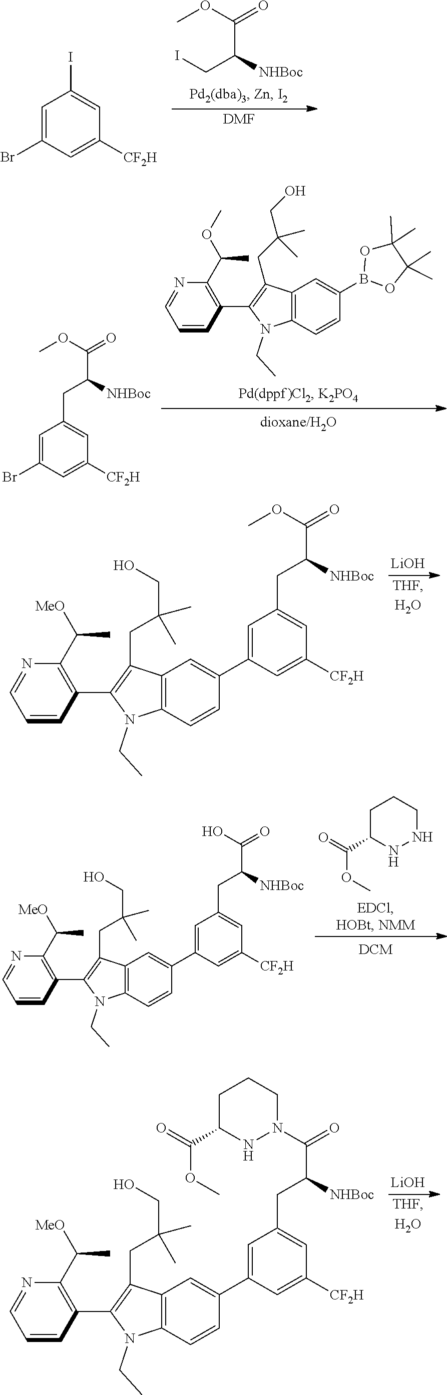

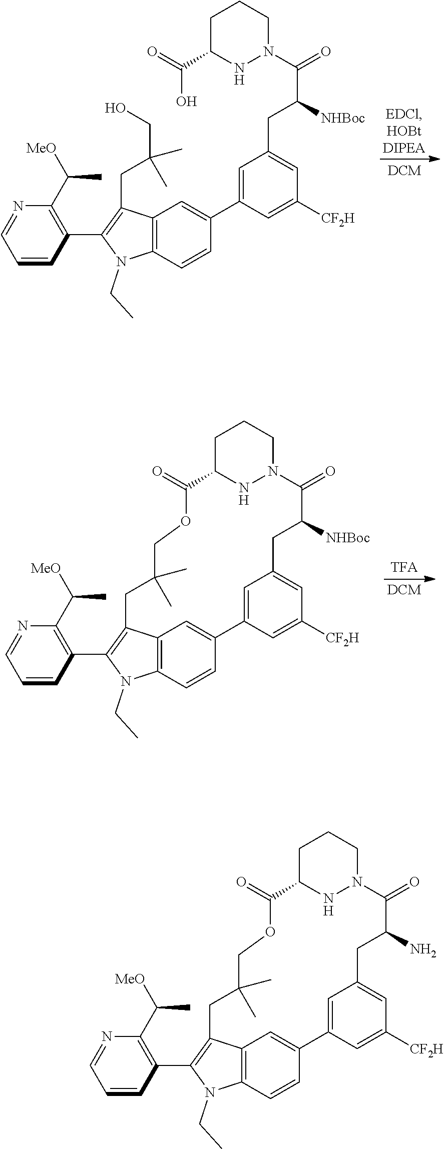

### Step 1: Synthesis of methyl (S)-3-(3-bromo-5-(difluoromethyl)phenyl)-2-((tert-butoxycarbonyl)amino)propanoate

Into a 1000 mL 3-necked round-bottom flask was added Zn powder (43.42 g, 663.835 mmol) and 12 (1.30 g, 5.106 mmol) in DMF (400 mL) at room temperature. To the above mixture was added a solution of methyl (2R)-2-[(tert-butoxycarbonyl)amino]-3-iodopropanoate (36.42 g, 110.64 mmol) in DMF (10 mL). The mixture was heated to 30° C. for 10 min. To the mixture was then added a solution of methyl (2R)-2-[(tert-butoxycarbonyl)amino]-3-iodopropanoate (72.83 g, 221.28 mmol) in DMF (20 mL) dropwise at room temperature. The resulting mixture was stirred for 30 min. The resulting mixture was filtered and the solution was added to a mixture of 1-bromo-3-(difluoromethyl)-5-iodobenzene (85.0 g, 255.321 mmol), tris(furan-2-yl) phosphane (3.56 g, 15.319 mmol), and Pd₂(dba)₃ (4.68 g, 5.106 mmol) in DMF (400 mL) at room temperature under argon atmosphere. The reaction mixture was heated to 60° C. for 10 min and was then removed from the oil bath and was stirred for 1 h until the temperature of the resulting mixture cooled down to 50° C. The reaction was quenched with aq. NH₄Cl (3000 mL) and the aqueous layer was extracted with EtOAc (3×1000 mL). The combined organic layers were washed with brine (2×1000 mL), dried over anhydrous Na₂SO₄, filtered, and concentrated under reduced pressure. The residue was purified by silica gel column chromatography (9% EtOAc/pet. ether) to afford the desired product (59 g, 56.6% yield).

### Step 2: Synthesis of methyl (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoate

A mixture of methyl (2S)-3-[3-bromo-5-(difluoromethyl)phenyl]-2-[(tert-butoxycarbonyl)amino]propanoate (90.0 g, 220.459 mmol), (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (1.50 g, 3.046 mmol), Pd(dppf)Cl₂ (16.13 g, 22.046 mmol) and K₃PO₄ (116.99 g, 551.148 mmol) in dioxane (600 mL), H₂O (200 mL), and toluene (200 mL) was stirred for 2 h at 70° C. The resulting mixture was concentrated under reduced pressure and then diluted with H₂O (300 mL). The mixture was extracted with EtOAc (3×500 mL). The combined organic layers were washed with H₂O (3×500 mL), dried over anhydrous Na₂SO₄. After filtration, the filtrate was concentrated under reduced pressure and the residue was purified by silica gel column chromatography (50% EtOAc/pet. ether) to afford the desired product (128 g, 83.7% yield). LCMS (ESI) m/z: [M+H] calcd for C₃₉H₄₉F₂N₃O₅: 694.37; found 694.2.

### Step 3: Synthesis of (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoic acid

To a stirred solution of methyl (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoate (125.0 g, 180.159 mmol) in THF (800 mL) was added LiOH·H₂O (11.48 g, 479.403 mmol) in H₂O (200 mL) dropwise at 0° C. The resulting mixture was stirred for 2 h at 0° C. The mixture was acidified to pH 6 with 1 M HCl (aq.) and was then extracted with EtOAc (3×800 mL). The combined organic layers were washed with brine (2×200 mL), dried over anhydrous Na₂SO₄. After filtration, the filtrate was concentrated under reduced pressure to afford the desired product (125 g, crude). LCMS (ESI) m/z: [M+H] calcd for C₃₆H₄₇F₂N₃O₆: 680.37; found 680.2.

### Step 4: Synthesis of methyl (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylate

To a stirred solution of methyl (3S)-1,2-diazinane-3-carboxylate (39.77 g, 275.814 mmol) and NMM (185.98 g, 1838.760 mmol) in DCM (1500 mL) was (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoic acid (125.0 g, 183.876 mmol), HOBt (12.42 g, 91.938 mmol) and EDCI (70.50 g, 367.752 mmol) in portions at 0° C. The resulting mixture was stirred at room temperature for 16 h. The reaction mixture was then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet.ether) to afford the desired product (110 g, 74.2% yield). LCMS (ESI) m/z: [M+H] calcd for C₄₄H₅₇F₂N₅O₇: 806.43; found 806.2.

### Step 5: Synthesis of (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylic acid

To a stirred solution of methyl (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylate (110.0 g, 136.482 mmol) in THF (800 mL) was added a solution of LiOH·H₂O (17.18 g, 409.446 mmol) in H₂O (200 mL) in portions at 0° C. The resulting mixture was stirred for 2 h at 0° C. and was then neutralized to pH 6 with 0.5 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (100 g, crude). LCMS (ESI) m/z: [M+H] calcd for C₄₃H₅₅F₂N₅O₇: 792.42; found 792.4.

### Step 6: Synthesis of tert-butyl ((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred solution of (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylic acid (100.0 g, 126.273 mmol) in DCM (6000 mL) was added DIPEA (163.20 g, 1262.730 mmol), HOBt (85.31 g, 631.365 mmol), and EDCI (363.10 g, 1894.095 mmol) dropwise at 0° C. The resulting mixture was stirred overnight at room temperature. The mixture was then washed with 0.5 M HCl (2×2000 mL) and brine (2×2000 mL), dried over anhydrous Na₂SO₄, filtered, and concentrated under reduced pressure. The residue was purified by silica gel column chromatography (50% EtOAc/pet. ether) to afford the desired product (70 g, 71.6% yield). LCMS (ESI) m/z: [M+H] calcd for C₄₃H₅₃F₂N₅O₆: 774.41; found 774.0.

### Step 7: Synthesis of (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred solution of tert-butyl ((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (202.0 mg, 0.261 mmol) in DCM (2 mL) was added TFA (1.0 mL) dropwise at 0° C. The resulting mixture was stirred for 1.5 h at 0° C. and was then concentrated under reduced pressure to afford the desired product. LCMS (ESI) m/z: [M+H] calcd for C₃₈H₄₅F₂N₅O₄: 674.35; found 674.5.

### Intermediate 29. Synthesis of (6³S4S)-4-amino-1¹-ethyl-2⁵-(fluoromethyl)-1²-(2-((S)-1-methoxyethyl)pyridin-3-yl)-10,10-dimethyl-6¹,6²,6³,6⁴,6⁵,6⁶-hexahydro-1¹H8-oxa-1(5,3)-indola-6(1,3)-pyridazina-2(1,3)-benzenacycloundecaphane-5,7-dione

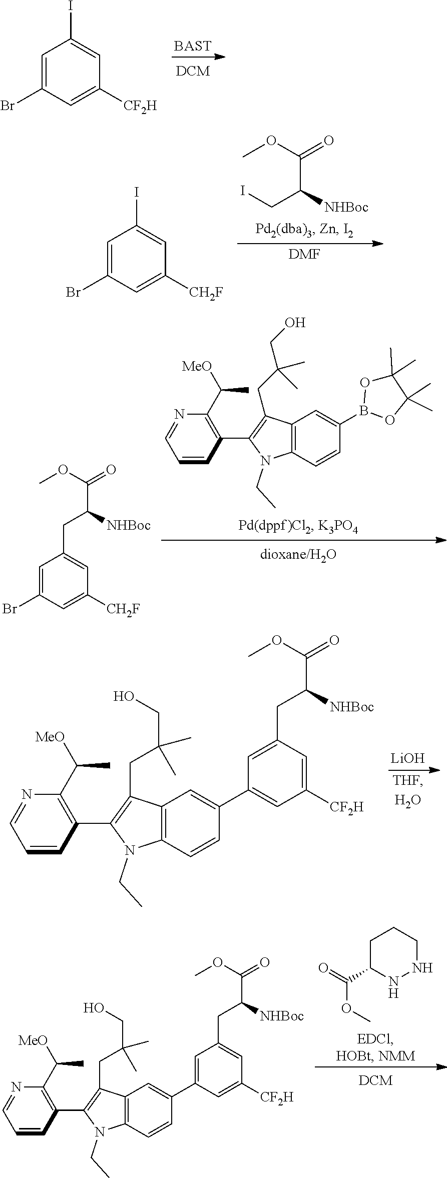

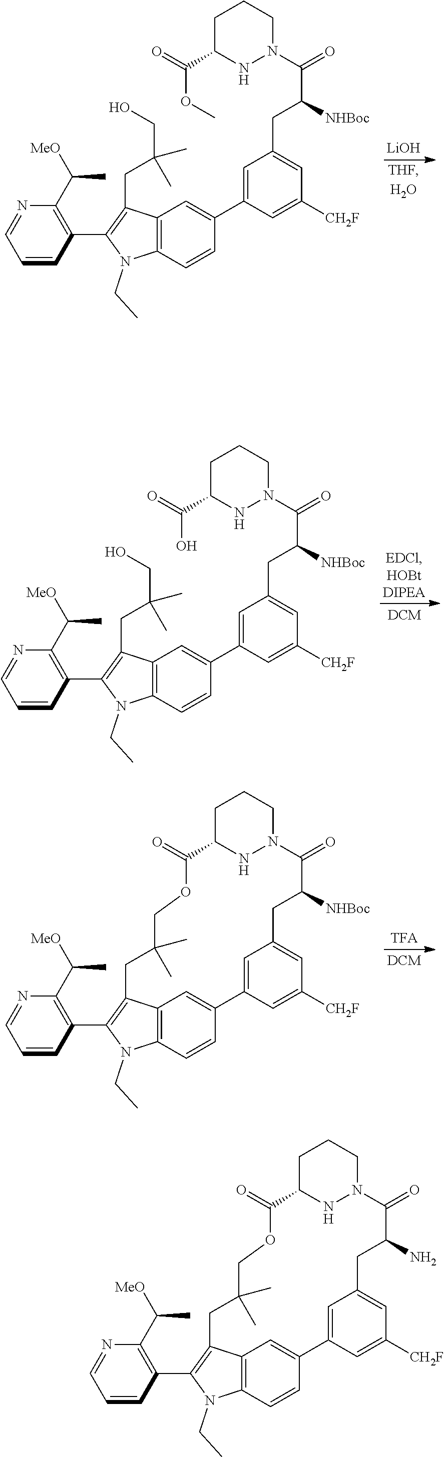

### Step 1: Synthesis of 1-bromo-3-(fluoromethyl)-5-iodobenzene

To a solution of (3-bromo-5-iodophenyl)methanol (175.0 g, 559.227 mmol) in DCM (2 L) was added BAST (247.45 g, 1118.454 mmol) dropwise at 0° C. The resulting mixture was stirred for 16 h at room temperature. The reaction was quenched with sat. aq. NaHCO₃ at 0° C. The organic layers were washed with H₂O (3×700 mL) and dried over anhydrous Na₂SO₄. After filtration, the filtrate was concentrated under reduced pressure. The residue was purified by silica gel column chromatography (3% EtOAc/pet. ether) to afford the desired product (120 g, 68% yield).

### Step 2: Synthesis of methyl (2S)-3-[3-bromo-5-(fluoromethyl)phenyl]-2-[(tert-butoxycarbonyl)amino]propanoate

Into a 1000 mL 3-necked round-bottom flask was added Zn powder (32.40 g, 495.358 mmol) in DMF (350.0 mL) and 12 (967.12 mg, 3.810 mmol). To the mixture was added a solution of methyl (2R)-2-[(tert-butoxycarbonyl)amino]-3-iodopropanoate (27.0 g, 82.03 mmol) in DMF (10 mL). The mixture was heated to 30° C. for 10 min. To the mixture was then added a solution of methyl (2R)-2-[(tert-butoxycarbonyl)amino]-3-iodopropanoate (54.0 g, 164.07 mmol) in DMF (20 mL). The resulting mixture was stirred for 30 min at room temperature and was filtered. The resulting solution was added to a mixture of 1-bromo-3-(fluoromethyl)-5-iodobenzene (60 g, 190.522 mmol), tris(furan-2-yl)phosphane (2.65 g, 11.431 mmol), and Pd₂(dba)₃ (3.49 g, 3.810 mmol) in DMF (400 mL) at room temperature under argon atmosphere and the reaction mixture was heated to 60° C. for 10 min then removed the oil bath. The resulting mixture was stirred for about 1 h until the temperature cooled down to 50° C. The reaction was quenched with aq. NH₄Cl (3000 mL) and the resulting mixture was extracted with EtOAc (3×1000 mL). The combined organic layers were washed with brine (2×1000 mL) and dried over anhydrous Na₂SO₄. After filtration, the filtrate was concentrated under reduced pressure. The residue was purified by silica gel column chromatography (9% EtOAc/pet. ether) to afford the desired product (45 g, 60% yield).

### Step 3: Synthesis of methyl (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoate

A mixture of methyl (2S)-3-[3-bromo-5-(fluoromethyl)phenyl]-2-[(tert-butoxycarbonyl)amino]propanoate (75.28 g, 192.905 mmol), (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (95 g, 192.905 mmol), Pd(dppf)Cl₂ (14.11 g, 19.291 mmol) and K₂CO₃ (53.32 g, 385.810 mmol) in dioxane (900 mL) and H₂O (180 mL) was stirred for 2 h at 80° C. The resulting mixture was concentrated under reduced pressure and was then diluted with H₂O. The resulting mixture was extracted with EtOAc (3×1200 mL) and the combined organic layers were washed with H₂O (3×500 mL) and dried over anhydrous Na₂SO₄. After filtration, the filtrate was concentrated under reduced pressure. The residue was purified by silica gel column chromatography (50% EtOAc/pet. ether) to afford the desired product (105 g, 80% yield). LCMS (ESI) m/z: [M+H] calcd for C₃₉H₅₀FN₃O₅: 676.38; found 676.1.

### Step 4: Synthesis of (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoic acid

To a stirred solution of methyl (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoate (108 g, 159.801 mmol) in THF (500 mL) was added a solution of LiOH·H₂O (11.48 g, 479.403 mmol) in H₂O (500 mL) at 0° C. The resulting mixture was stirred for 2 h at 0° C. and was then acidified to pH 6 with 1 M HCl (aq.). The mixture was extracted with EtOAc (3×800 mL) and the combined organic layers were washed with brine (2×200 mL) and dried over anhydrous Na₂SO₄. After filtration, the filtrate was concentrated under reduced pressure to afford the desired product (101 g, crude). LCMS (ESI) m/z: [M+H] calcd for C₃₈H₄₈FN₃O₆: 662.36; found 662.1.

### Step 5: Synthesis of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylate

To a stirred solution of (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoic acid (103 g, 155.633 mmol) and NMM (157.42 g, 1556.330 mmol) in DCM (1200 mL) was added methyl (3S)-1,2-diazinane-3-carboxylate (33.66 g, 233.449 mmol), HOBt (10.51 g, 77.816 mmol) and EDCI (59.67 g, 311.265 mmol) in portions at 0° C. The resulting mixture was stirred a t room temperature for 16 h. The organic layers were then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet. ether) to afford the desired product (103 g, 83% yield). LCMS (ESI) m/z: [M+H] calcd for C₄₄H₅₈FN₅O₇: 788.44; found 788.1.

### Step 6: Synthesis of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylic acid

To a stirred solution of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylate (103 g, 130.715 mmol) in THF (700 mL) was added a solution of LiOH·H₂O (27.43 g, 653.575 mmol) in H₂O (700 mL) at 0° C. The resulting mixture was stirred for 2 h at 0° C. and was then neutralized to pH 6 with 1 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (101 g, crude). LCMS (ESI) m/z: [M+H] calcd for C₄₃H₅₆FN₅O₇: 774.43; found 774.1.

### Step 7: Synthesis of tert-butyl ((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred solution of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylic acid (101 g, 130.50 mmol) in DCM (5500 mL) was added DIPEA (227.31 mL, 1305.0 mmol) and HOBt (88.17 g, 652.499 mmol), and EDCI (375.26 g, 1957.498 mmol) at 0° C. The resulting mixture was stirred at room temperature overnight. The mixture was then washed with 0.5 M HCl (2×2000 mL), brine (2×2000 mL), dried over anhydrous Na₂SO₄, filtered, and concentrated under reduced pressure. The residue was purified by silica gel column chromatography (50% EtOAc/pet. ether) to afford the desired product (68 g, 65% yield). LCMS (ESI) m/z: [M+H] calcd for C₄₃H₅₄FN₅O₆: 756.42; found 756.4.

### Step 8: Synthesis of (2S)—N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide

To a stirred solution of tert-butyl ((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 0.403 mmol) in DCM (4 mL) was added TFA (1.50 mL) at 0° C. The resulting mixture was stirred at room temperature for 1.5 h and was then concentrated under reduced pressure to afford the desired product (600 mg, crude). LCMS (ESI) m/z: [M+H] calcd for C₃₈H₄₆FN₅O₄: 656.36; found 656.4.

### Intermediate A-1. Synthesis of N-methyl-N—((S)-1-((R)-1-tritylaziridine-2-carbonyl)pyrrolidine-3-carbonyl)-L-valine

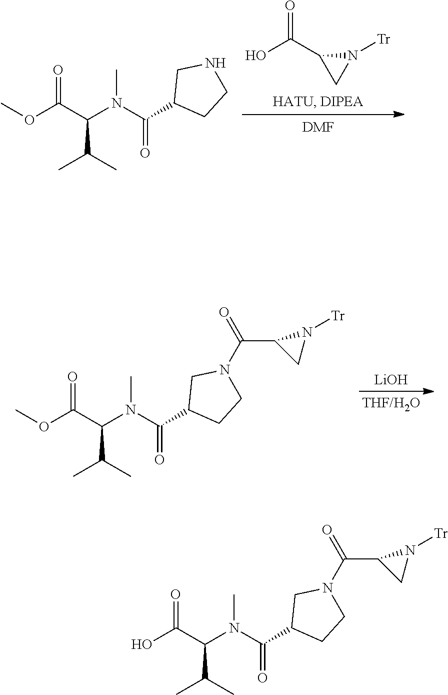

### Step 1: Synthesis of methyl N-methyl-N—((S)-1-((R)-1-tritylaziridine-2-carbonyl)pyrrolidine-3-carbonyl)-L-valinate

To a mixture of methyl N-methyl-N—((S)-pyrrolidine-3-carbonyl)-L-valinate (0.840 g, 3.47 mmol) and (R)-1-tritylaziridine-2-carboxylic acid (1.713 g, 5.2 mmol) in DMF (20 mL) at 0° C. was added DIPEA (3.0 mL, 17.33 mmol) and HATU (2.636 g, 6.93 mmol). The reaction mixture was stirred for 3 h, at which point the mixture was extracted with EtOAc (200 mL). The EtOAc layer was washed with brine (3×50 mL), dried over Na₂SO₄, filtered, and concentrated under reduced pressure. The crude residue was purified by reverse phase chromatography (10→50% MeCN/H₂O) to afford the desired product (1.02 g, 53% yield) as a solid. LCMS (ESI) m/z: [M+H] calcd for C₃₄H₃₉N₃O₄: 554.30; found 554.3.

### Step 2: Synthesis of N-methyl-N—((S)-1-((R)-1-tritylaziridine-2-carbonyl)pyrrolidine-3-carbonyl)-L-valine

To a solution of methyl N-methyl-N—((S)-1-((R)-1-tritylaziridine-2-carbonyl)pyrrolidine-3-carbonyl)-L-valinate (1.0 g, 1.81 mmol) in THF (10 mL) at 0° C. was added a solution of LiOH·H₂O (0.3789 g, 9.03 mmol) in H₂O (9.0 mL). After 3 h, the reaction solution was neutralized to pH 7 with sat. aq. NH₄Cl. The resulting mixture was extracted with EtOAc (3×50 mL) and the combined organic layers were washed with brine (3×20 mL), dried over Na₂SO₄, filtered, and concentrated under reduced pressure to afford the crude product (740 mg, 75.9% yield) as a solid. LCMS (ESI) m/z: [M−H] calcd for C₃₃H₃₇N₃O₄: 538.27; found 538.2.

### Intermediate A-2. Synthesis of N-methyl-N—((S)-1-((S)-1-tritylaziridine-2-carbonyl)pyrrolidine-3-carbonyl)-L-valine

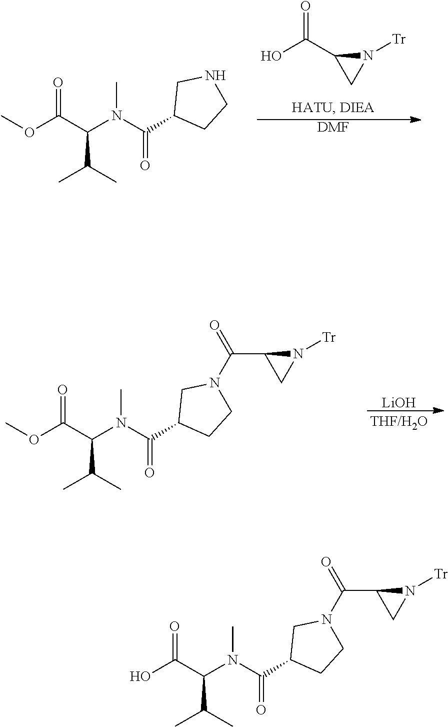

### Step 1: Synthesis of methyl N-methyl-N—((S)-1-((S)-1-tritylaziridine-2-carbonyl)pyrrolidine-3-carbonyl)-L-valinate

To a mixture of methyl N-methyl-N—((S)-pyrrolidine-3-carbonyl)-L-valinate (0.800 g, 3.30 mmol) and (S)-1-tritylaziridine-2-carboxylic acid (1.305 g, 3.96 mmol) in DMF (16 mL) at 0° C. was added DIPEA (2.9 mL, 16.5 mmol) and HATU (1.88 g, 4.9 mmol). The reaction mixture was warmed to room temperature and stirred for 1 h, at which point the mixture was diluted with EtOAc. The mixture was washed with sat. NH₄Cl and the resulting aqueous layer extracted with EtOAc. The combined organic layers were washed with brine, dried over Na₂SO₄, filtered, and concentrated under reduced pressure. The crude residue was purified by reverse phase chromatography (10→80% MeCN/H₂O) to afford the desired product (1.17 g, 64% yield) as a solid. LCMS (ESI) m/z: [M+H] calcd for C₃₄H₃₉N₃O₄: 554.30; found 554.3.

### Step 2: Synthesis of N-methyl-N—((S)-1-((S)-1-tritylaziridine-2-carbonyl)pyrrolidine-3-carbonyl)-L-valine

To a stirred solution of methyl N-methyl-N—((S)-1-((S)-1-tritylaziridine-2-carbonyl)pyrrolidine-3-carbonyl)-L-valinate (1.10 g, 1.99 mmol) in THF (10.0 mL) at 0° C. was added a 1M solution of LiOH (9.93 mL, 9.93 mmol). The reaction mixture was warmed to room temperature and stirred for 16 h. The reaction mixture was cooled to 0° C. and quenched with sat. aq. NH₄Cl to pH 6. The resulting mixture was extracted with EtOAc. The combined organic layers were washed with brine, dried over Na₂SO₄, filtered, and concentrated under reduced pressure to afford the desired crude product (1.2 g). LCMS (ESI) m/z: [M+H] calcd for C₃₃H₃₇N₃O₄: 540.29; found 540.3.

### Intermediate A-3. Synthesis of N-methyl-N-(1-((R)-1-tritylaziridine-2-carbonyl)piperidine-4-carbonyl)-L-valine

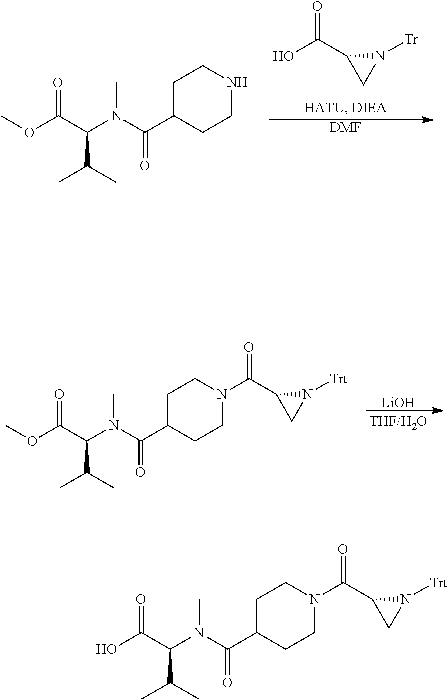

### Step 1: Synthesis of methyl N-methyl-N-(1-((H)-1-tritylaziridine-2-carbonyl)piperidine-4-carbonyl)-L-valinate

To a solution of (R)-1-tritylaziridine-2-carboxylic acid (1.157 g, 3.51 mmol) and methyl N-methyl-N-(piperidine-4-carbonyl)-L-valinate (0.600 g, 2.34 mmol) in DMF (20 mL) at 0° C. was added DIPEA (0.204 mL, 11.70 mmol) and HATU (1.780 g, 4.68 mmol. After 3 h, the reaction mixture was extracted with EtOAc (200 mL). The combined organic layers were washed with brine (3×50 mL), dried over Na₂SO₄, filtered, and concentrated under reduced pressure. The residue was purified by reverse phase chromatography (10→50% MeCN/H₂O) to afford the desired product (740 mg, 55.7% yield) as a solid. LCMS (ESI) m/z: [M+H] calcd for C₃₅H₄₁N₃O₄: 568.32; found 568.3.

### Step 2: Synthesis of N-methyl-N-(1-((R)-1-tritylaziridine-2-carbonyl)piperidine-4-carbonyl)-L-valine

To a solution of methyl N-methyl-N-(1-((R)-1-tritylaziridine-2-carbonyl)piperidine-4-carbonyl)-L-valinate (0.700 g, 1.23 mmol) in THF (7.0 mL) at 0° C. was added a solution of LiOH·H₂O (0.259 g, 6.17 mmol) in H₂O (6.0 mL). The resulting solution was warmed to room temperature and stirred for 3 h. The reaction mixture was diluted with EtOAc (100 mL) and was washed with sat. brine (5×50 mL). The organic layer was dried over Na₂SO₄, filtered, and concentrated under reduced pressure to afford the crude product (700 mg) as a solid. LCMS (ESI) m/z: [M−H] calcd for C₃₄H₃₉N₃O₄: 552.29; found 552.2.

### Intermediate A-4. Synthesis of N-methyl-N-(1-((S)-1-tritylaziridine-2-carbonyl)piperidine-4-carbonyl)-L-valine

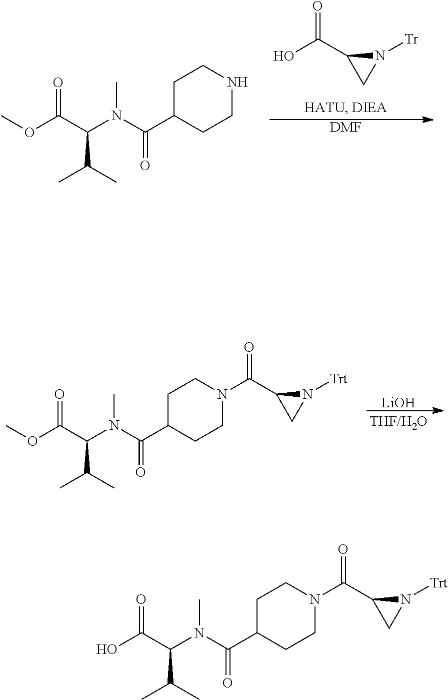

### Step 1: Synthesis of methyl N-methyl-N-(1-((S)-1-tritylaziridine-2-carbonyl)piperidine-4-carbonyl)-L-valinate

To a solution of methyl N-methyl-N-(piperidine-4-carbonyl)-L-valinate (0.550 g, 2.15 mmol) and (S)-1-tritylaziridine-2-carboxylic acid (0.848 g, 2.57 mmol) in DMF (10.0 mL) at 0° C. was added DIPEA (1.9 mL, 10.7 mmol) and HATU (1.2 g, 3.2 mmol). The reaction mixture was warmed to room temperature and stirred for 1 h. The reaction mixture was diluted with EtOAc (50 mL) and washed with sat. NH₄Cl (60 mL). The aqueous layer was extracted with EtOAc (3×50 mL). The combined organic layers were washed with brine (200 mL), dried over Na₂SO₄, filtered, and concentrated under reduced pressure. The residue was purified by reverse phase chromatography (10→80% MeCN/H₂O) to afford the desired product (1.2 g, 98.5% yield) as a solid. LCMS (ESI) m/z: [M+H] calcd for C₃₅H₄₁N₃O₄: 568.32; found 568.3.

### Step 2: Synthesis of N-methyl-N-(1-((S)-1-tritylaziridine-2-carbonyl)piperidine-4-carbonyl)-L-valine

To a solution of methyl N-methyl-N-(1-((S)-1-tritylaziridine-2-carbonyl)piperidine-4-carbonyl)-L-valinate (1.20 g, 2.11 mmol) in THF (11.0 mL) at 0° C. was added 1M LiOH (10.57 mL, 10.57 mmol). The resulting solution was warmed to room temperature and stirred for 16 h. The reaction mixture was cooled to 0° C. and quenched with sat. NH₄Cl until pH 6. The resulting mixture was extracted with EtOAc (3×50 mL) and the combined organic layers were washed with brine (3×50 mL), dried over Na₂SO₄, filtered, and concentrated under reduced pressure to afford the crude product (900 mg). LCMS (ESI) m/z: [M−H] calcd for C₃₄H₃₉N₃O₄: 554.29; found 554.3.

### Intermediate A-5. Synthesis of N-methyl-N-(1-((R)-1-tritylaziridine-2-carbonyl)azetidine-3-carbonyl)-L-valine

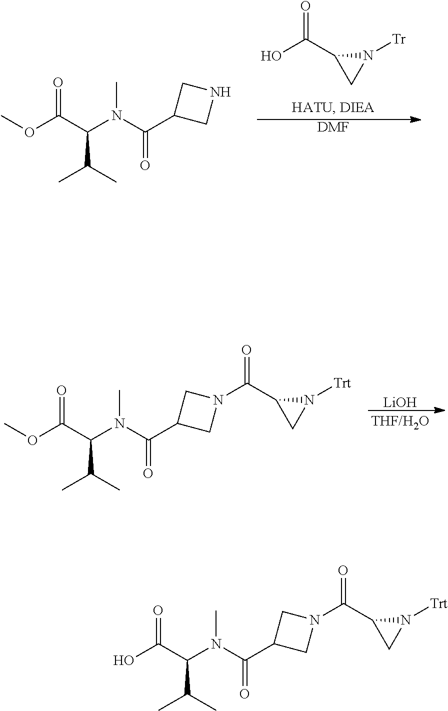

### Step 1: Synthesis of methyl N-methyl-N-(1-((R)-1-tritylaziridine-2-carbonyl)azetidine-3-carbonyl)-L-valinate

To a solution of methyl N-(azetidine-3-carbonyl)-N-methyl-L-valinate (0.410 g, 1.79 mmol) and (R)-1-tritylaziridine-2-carboxylic acid (0.887 g, 2.69 mmol) in DMF (10 mL) at 0° C. was added DIPEA (1.56 mL, 8.98 mmol) and HATU (1.37 g, 3.59 mmol). The reaction mixture was stirred for 1 h. The resulting mixture was then extracted with EtOAc (3×50 mL) and the combined organic layers were washed with brine (3×20 mL), dried over Na₂SO₄, filtered, and concentrated under reduced pressure. The residue was purified by reverse phase chromatography (10→80% MeCN/H₂O) to afford the desired product (650 mg, 67% yield) as a solid. LCMS (ESI) m/z: [M+H] calcd for C₃₃H₃₇N₃O₄: 540.29; found 540.3.

### Step 2: Synthesis of N-methyl-N-(1-((R)-1-tritylaziridine-2-carbonyl)azetidine-3-carbonyl)-L-valine

To a solution of methyl N-methyl-N-(1-((R)-1-tritylaziridine-2-carbonyl)azetidine-3-carbonyl)-L-valinate (0.650 mg, 1.20 mmol) in THF (10 mL) at 0° C. was added a 1M solution of LiOH·H₂O (6.03 mL). The reaction mixture was stirred for 3 h. The resulting mixture was then quenched with sat. NH₄Cl until pH 7. The resulting mixture was extracted with EtOAc (3×20 mL) and the combined organic layers were washed with brine (3×20 mL), dried over Na₂SO₄, filtered, and concentrated under reduced pressure to afford the desired crude product (588 mg) as a solid. LCMS (ESI) m/z: [M−H] calcd for C₃₂H₃₅N₃O₄: 526.27; found 526.3.

### Intermediate A-6. Synthesis of N-methyl-N-(1-((S)-1-tritylaziridine-2-carbonyl)azetidine-3-carbonyl)-L-valine

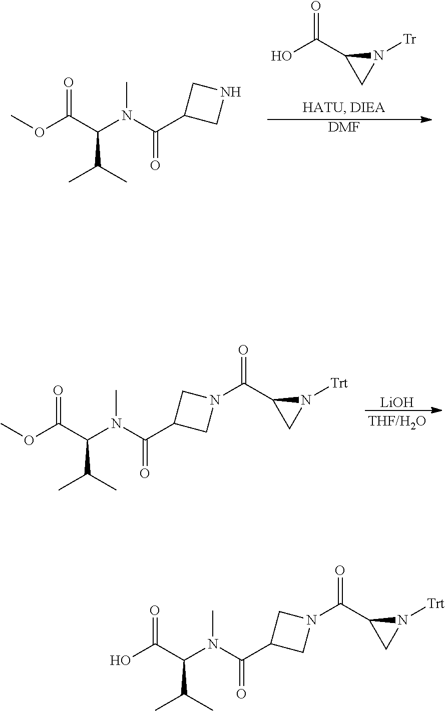

### Step 1: Synthesis of methyl N-methyl-N-(1-((S)-1-tritylaziridine-2-carbonyl)azetidine-3-carbonyl)-L-valinate

To a solution of methyl N-(azetidine-3-carbonyl)-N-methyl-L-valinate (0.550 g, 2.41 mmol) and (S)-1-tritylaziridine-2-carboxylic acid (0.952 g, 2.89 mmol) in DMF (10 mL) at 0° C. was added DIPEA (2.1 mL, 12.05 mmol) and HATU (1.37 g, 3.61 mmol). The reaction mixture was warmed to room temperature and stirred for 1 h. The resulting mixture was diluted with EtOAc (50 mL) and washed with sat. NH₄Cl (60 mL). The aqueous layer was then extracted with EtOAc (3×50 mL) and the combined organic layers were washed with brine (200 mL), dried over Na₂SO₄, filtered, and concentrated under reduced pressure. The residue was purified by reverse phase chromatography (10→80% MeCN/H₂O) to afford the desired product (820 mg, 63% yield) as a solid. LCMS (ESI) m/z: [M+H] calcd for C₃₃H₃₇N₃O₄: 540.29; found 540.3.

### Step 2: Synthesis of N-methyl-N-(1-((S)-1-tritylaziridine-2-carbonyl)azetidine-3-carbonyl)-L-valine

To a solution of methyl N-methyl-N-(1-((S)-1-tritylaziridine-2-carbonyl)azetidine-3-carbonyl)-L-valinate (0.800 g, 1.48 mmol) in THF (8.0 mL) at 0° C. was added 1M LiOH (7.41 mL, 7.41 mmol). The reaction mixture was warmed to room temperature and stirred for 16 h and was then cooled to 0° C. and quenched with sat. NH₄Cl until pH 6. The resulting mixture was extracted with EtOAc (3×50 mL) and the combined organic layers were washed with brine (150 mL), dried over Na₂SO₄, filtered, and concentrated under reduced pressure. The residue was purified by reverse phase chromatography (10→80% MeCN/H₂O+0.5% NH₄HCO₃) to afford the desired product (440 mg, 56% yield) as a solid. LCMS (ESI) m/z: [M−H] calcd for C₃₂H₃₅N₃O₄: 524.25; found 524.2.

### Intermediate A-7. Synthesis of (2R,3S)-3-phenylaziridine-2-carboxylic acid

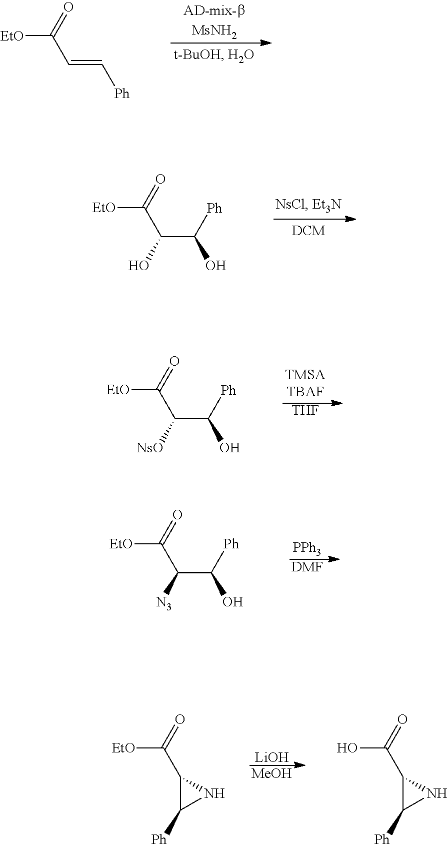

### Step 1: Synthesis of ethyl (2S,3R)-2,3-dihydroxy-3-phenylpropanoate

To a solution of ethyl cinnamate (2.0 g, 11.4 mmol) in t-BuOH (35.0 mL) and H₂O (35.0 mL) at 0° C. was added AD-mix-β (15.83 g, 20.32 mmol), and methanesulfonamide (1.08 g, 11.3 mmol). The reaction mixture was stirred at room temperature for 16 h. The reaction was cooled to 0° C. and quenched with aq. KHSO₄. The resulting mixture was extracted with EtOAc (3×100 mL) and the combined organic layers were washed with brine (2×90 mL), dried over Na₂SO₄, filtered, and concentrated under reduced pressure. The residue was purified by normal phase chromatography (50% EtOAc/pet. ether) to afford the desired product (2.2 g, 82% yield) as a solid.

### Step 2: Synthesis of ethyl (2S,3R)-3-hydroxy-2-(((4-nitrophenyl)sulfonyl)oxy)-3-phenylpropanoate

To a solution of ethyl (2S,3R)-2,3-dihydroxy-3-phenylpropanoate (2.0 g, 9.5 mmol) and Et₃N (3.97 mL, 28.5 mmol) in DCM (30.0 mL) at 0° C. was added 4-nitrobenzenesulfonyl chloride (2.11 g, 9.51 mmol). The resulting mixture was stirred for 1 h and was then diluted with H₂O (300 mL). The mixture was extracted with DCM (3×100 mL) and the combined organic layers were washed with brine (2×100 mL), dried over Na₂SO₄, filtered, and concentrated under reduced pressure. The residue was purified by prep-TLC (50% EtOAc/pet. ether) to afford the desired product (2.8 g, 67% yield) as a solid.

### Step 3: Synthesis of ethyl (2R,3R)-2-azido-3-hydroxy-3-phenylpropanoate

To a solution of ethyl (2S,3R)-3-hydroxy-2-(((4-nitrophenyl)sulfonyl)oxy)-3-phenylpropanoate (2.80 g, 7.08 mmol) in THF (30 mL) at room temperature was added trimethylsilyl azide (1.63 g, 14.2 mmol) and TBAF (1M in THF, 14.16 mL, 14.16 mmol). The reaction mixture was heated to 60° C. and was stirred for 16 h. The reaction mixture was then cooled to room temperature, diluted with H₂O (150 mL), and extracted with EtOAc (3×50 mL). The combined organic layers were washed with brine (2×30 mL), dried over Na₂SO₄, filtered, and concentrated under reduced pressure. The residue was purified by normal phase chromatography (50%/a EtOAc/pet. ether) to afford the desired product (1.2 g, 64% yield) as an oil.

### Step 4: Synthesis of ethyl (2R,3S)-3-phenylaziridine-2-carboxylate

To a solution of ethyl (2R,3R)-2-azido-3-hydroxy-3-phenylpropanoate (1.20 g, 5.10 mmol) in DMF (15.0 mL) was added PPh₃ (1.61 g, 6.12 mmol). The reaction mixture was stirred at room temperature for 30 min and then heated to 80° C. for an additional 16 h. The reaction mixture was then cooled to room temperature, diluted with H₂O (100 mL), and extracted with EtOAc (3×40 mL). The combined organic layers were washed with brine (20 mL), dried over Na₂SO, filtered, and concentrated under reduced pressure. The residue was purified by normal phase chromatography (16% EtOAc/pet. ether) to afford the desired product (620 mg, 57% yield) as an oil. LCMS (ESI) m/z: [M+H] calcd for C₁₁H₁₃NO₂: 192.10; found 192.0.

### Step 5: Synthesis of (2R,3S)-3-phenylaziridine-2-carboxylic acid

To a solution of ethyl (2R,3S)-3-phenylaziridine-2-carboxylate (0.100 g, 0.523 mmol) in MeOH (0.70 mL) at 0° C. was added a solution of LiOH (18.8 mg, 0.784 mmol) in H₂O (0.70 mL). The reaction mixture was stirred for 1 h. The mixture was then diluted with MeCN (10 mL), and the resulting precipitate was collected by filtration and washed with MeCN (2×10 mL) to afford the crude desired product (70 mg) as a solid. LCMS (ESI) m/z: [M+H] calcd for C₉H₉NO₂: 164.07; found 164.0.

### Intermediate A-8. Synthesis of (2S,3R)-3-phenylaziridine-2-carboxylic acid

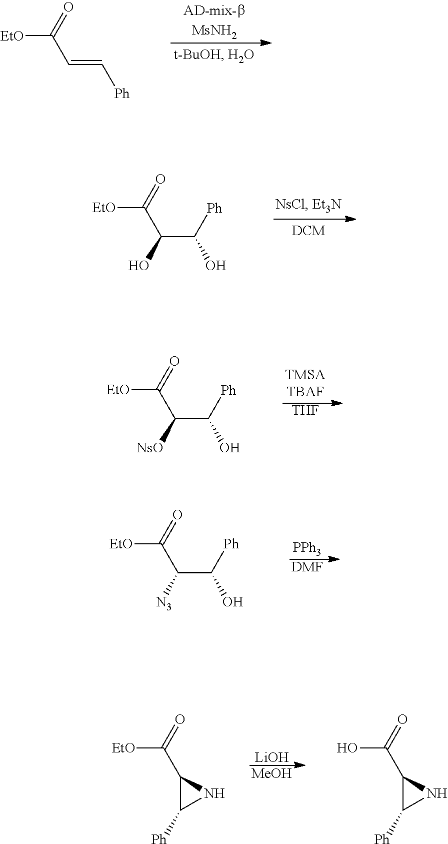

### Step 1: Synthesis of ethyl (2R,3S)-2,3-dihydroxy-3-phenylpropanoate

To a solution of ethyl cinnamate (2.0 g, 11.4 mmol) in t-BuOH (35.0 mL) and H₂O (35.0 mL) at 0° C. was added AD-mix-α (15.83 g, 20.32 mmol), and methanesulfonamide (1.08 g, 11.3 mmol). The reaction mixture was stirred at room temperature for 16 h. The reaction was cooled to 0° C. and quenched with aq. KHSO₄. The resulting mixture was extracted with EtOAc (3×100 mL) and the combined organic layers were washed with brine (2×80 mL), dried over Na₂SO₄, filtered, and concentrated under reduced pressure. The residue was purified by normal phase chromatography (50% EtOAc/pet. ether) to afford the desired product (2.2 g, 82% yield) as a solid.

### Step 2: Synthesis of ethyl (2R,3S)-3-hydroxy-2-(((4-nitrophenyl)sulfonyl)oxy)-3-phenylpropanoate

To a solution of ethyl (2R,3S)-2,3-dihydroxy-3-phenylpropanoate (2.10 g, 9.99 mmol) and Et₃N (4.18 mL, 29.9 mmol) in DCM (30.0 mL) at 0° C. was added 4-nitrobenzenesulfonyl chloride (2.21 g, 9.99 mmol). The resulting mixture was stirred for 1 h and was then diluted with H₂O (200 mL). The mixture was extracted with DCM (3×80 mL) and the combined organic layers were washed with brine (2×80 mL), dried over Na₂SO₄, filtered, and concentrated under reduced pressure. The residue was purified by prep-TLC (50% EtOAc/pet. ether) to afford the desired product (3.0 g, 68% yield) as a solid.

### Step 3 Synthesis of ethyl (2S,3S)-2-azido-3-hydroxy-3-phenylpropanoate

To a solution of ethyl (2R,3S)-3-hydroxy-2-(((4-nitrophenyl)sulfonyl)oxy)-3-phenylpropanoate (3.0 g, 7.59 mmol) in THF (30 mL) at room temperature was added trimethylsilyl azide (1.75 g, 15.2 mmol) and TBAF (1M in THF, 15.18 mL, 15.18 mmol). The reaction mixture was heated to 60° C. and was stirred for 16 h. The reaction mixture was then cooled to room temperature, diluted with H₂O (150 mL), and extracted with EtOAc (3×50 mL). The combined organic layers were washed with brine (2×30 mL), dried over Na₂SO₄, filtered, and concentrated under reduced pressure. The residue was purified by normal phase chromatography (50% EtOAc/pet. ether) to afford the desired product (1.4 g, 70% yield) as an oil.

### Step 4: Synthesis of ethyl (2S,3R)-3-phenylaziridine-2-carboxylate

To a solution of ethyl (2S,3S)-2-azido-3-hydroxy-3-phenylpropanoate (1.40 g, 5.95 mmol) in DMF (20.0 mL) was added PPh₃ (1.87 g, 7.14 mmol). The reaction mixture was stirred at room temperature for 30 min and then heated to 80° C. for an additional 16 h. The reaction mixture was then cooled to room temperature, diluted with H₂O (150 mL), and extracted with EtOAc (3×50 mL). The combined organic layers were washed with brine (40 mL), dried over Na₂SO, filtered, and concentrated under reduced pressure. The residue was purified by normal phase chromatography (16% EtOAc/pet. ether) to afford the desired product (720 mg, 56% yield) as an oil. LCMS (ESI) m/z: [M+H] calcd for C₁₁H₁₃NO₂: 192.10; found 192.0.

### Step 5: Synthesis of (2S,3R)-3-phenylaziridine-2-carboxylic acid

To a solution of ethyl (2S,3R)-3-phenylaziridine-2-carboxylate (0.100 g, 0.523 mmol) in MeOH (0.70 mL) at 0° C. was added a solution of LiOH (18.8 mg, 0.784 mmol) in H₂O (0.70 mL). The reaction mixture was stirred for 1 h. The mixture was then diluted with MeCN (10 mL), and the resulting precipitate was collected by filtration and washed with MeCN (2×10 mL) to afford the crude desired product (68 mg) as a solid. LCMS (ESI) m/z: [M+H] calcd for C₉H₉NO₂: 164.07; found 164.0.

### Intermediate A-9. Synthesis of N—(N—((R)-1-benzylaziridine-2-carbonyl)-N-methylglycyl)-N-methyl-L-valine

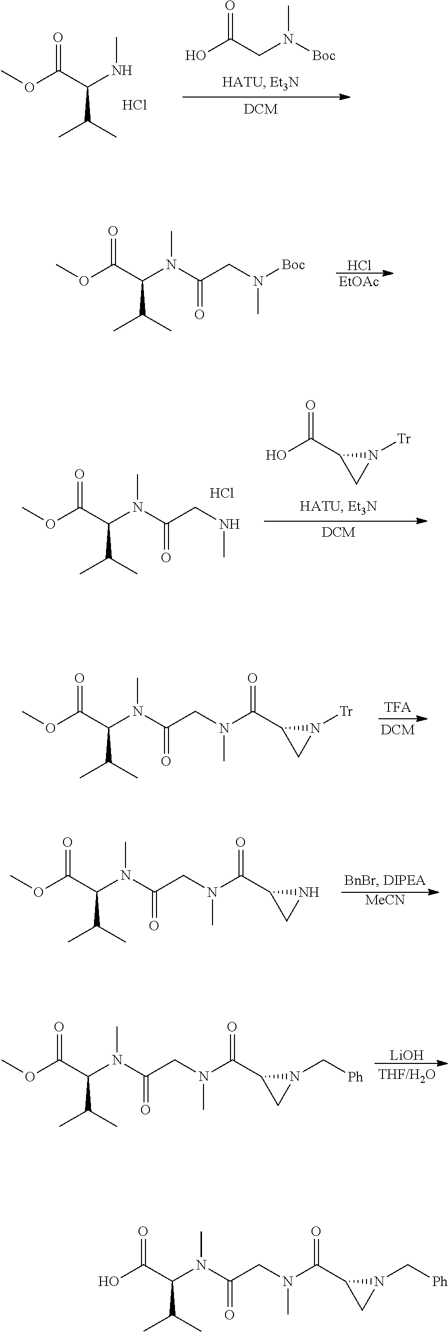

### Step 1: Synthesis of methyl N—(N-(tert-butoxycarbonyl)-N-methylglycyl)-N-methyl-L-valinate

To a solution of methyl methyl-L-valinate hydrochloride (4.0 g, 22.0 mmol) and N-(tert-butoxycarbonyl)-N-methylglycine (5.0 g, 26.4 mmol) in DCM (100.0 mL) was added Et₃N (9.2 mL, 66.1 mmol) and HATU (10.88 g, 28.63 mmol). The reaction mixture was stirred for 4 h. The reaction was then neutralized to pH 7 with sat. aq. NaHCO₃. The mixture was extracted with DCM and the combined organic layers were washed with brine, dried over Na₂SO₄, filtered, and concentrated under reduced pressure. The residue was purified by normal phase chromatography (50% EtOAc/pet. ether) to afford the desired product (6.2 g, 89% yield) as an oil. LCMS (ESI) m/z: [M+H] calcd for C₁₅H₂₈N₂O₅: 317.21; found 317.2.

### Step 2: Synthesis of methyl N-methyl-N-(methylglycyl)-L-valinate hydrochloride

To a solution of methyl N—(N-(tert-butoxycarbonyl)-N-methylglycyl)-N-methyl-L-valinate (4.97 g, 15.7 mmol) in EtOAc (150.0 mL) at 0° C. was added HCl (4M in dioxane, 50.0 mL, 200 mmol). The reaction mixture was stirred for 3 h and then concentrated under reduced pressure to afford the desired crude product (4.26 g, 107% yield) as an oil. LCMS (ESI) m/z: [M+H] calcd for C₁₀H₂₀N₂O₃: 217.16; found 217.1.

### Step 3: Synthesis of methyl N-methyl-N—(N-methyl-N—((R)-1-tritylaziridine-2-carbonyl)glycyl)-L-valinate

To a solution of methyl N-methyl-N-(methylglycyl)-L-valinate hydrochloride (1.0 g, 3.9 mmol) and (R)-1-tritylaziridine-2-carboxylic acid (1.30 g, 3.94 mmol) in DCM (25.0 mL) was added Et₃N (2.76 mL, 19.8 mmol) and HATU (1.81 g, 4.76 mmol). The reaction mixture was stirred for 1 h. The reaction was then neutralized to pH 7 with sat. aq. NaHCO₃. The mixture was extracted with DCM and the combined organic layers were washed with brine, dried over Na₂SO₄, filtered, and concentrated under reduced pressure. The residue was purified by normal phase chromatography (50% EtOAc/pet. ether) to afford the desired product (1.1 g, 52.6% yield) as a solid. LCMS (ESI) m/z: [M+H] calcd for C₃₂H₃₇N₃O₄: 528.29; found 528.2.

### Step 4: Synthesis of methyl N—(N—((R)-aziridine-2-carbonyl)-N-methylglycyl)-N-methyl-L-valinate

To a solution methyl N-methyl-N—(N-methyl-N—((R)-1-tritylaziridine-2-carbonyl)glycyl)-L-valinate (1.0 g, 3.9 mmol) in DCM (6 mL) at 0° C. was added TFA (2 mL). The reaction mixture was warmed to room temperature and stirred for 1 h, then concentrated under reduced pressure to afford the desired crude product (250 mg) as an oil. LCMS (ESI) m/z: [M+H] calcd for C₁₃H₂₃N₃O₄: 286.18; found 286.1.

### Step 5: Synthesis of methyl N—(N—((R)-1-benzylaziridine-2-carbonyl)-N-methylglycyl)-N-methyl-L-valinate

To a solution of methyl N—(N—((R)-aziridine-2-carbonyl)-N-methylglycyl)-N-methyl-L-valinate (220.0 mg, 0.771 mmol) in MeCN (2.0 mL) was added DIPEA (537 μL, 3.08 mmol) and benzyl bromide (101 μL, 0.848 mmol). The reaction mixture was stirred for 6 h. The reaction mixture was then concentrated under reduced pressure. The residue was purified by prep-TLC (9% MeOH/DCM) to afford the desired product (261 mg, 90% yield) as an oil. LCMS (ESI) m/z: [M+H] calcd for C₂₀H₂₉N₃O₄: 376.22; found 376.2.

### Step 6: Synthesis of N—(N—((R)-1-benzylaziridine-2-carbonyl)-N-methylglycyl)-N-methyl-L-valine

To a solution of methyl N—(N—((R)-1-benzylaziridine-2-carbonyl)-N-methylglycyl)-N-methyl-L-valinate (261.0 mg, 0.695 mmol) in THF (3.38 mL) was added a solution of LiOH (83.2 mg, 3.48 mmol) in H₂O (3.50 mL). The reaction mixture was stirred for 1 h. The reaction was then quenched with sat. aq. NH₄Cl. The resulting mixture was extracted with EtOAc and the combined organic layers were washed with brine, dried over Na₂SO₄, filtered, and concentrated under reduced pressure. The residue was purified by reverse phase chromatography (10→50% MeCN/H₂O) to afford the desired product (230 mg, 91% yield) as an oil. LCMS (ESI) m/z: [M+H] calcd for C₁₉H₂₇N₃O₄: 362.21; found 362.2.

### Intermediate A-10. Synthesis of N—(N—((R)-1-benzylaziridine-2-carbonyl)-N-methylglycyl)-N-methyl-L-valine

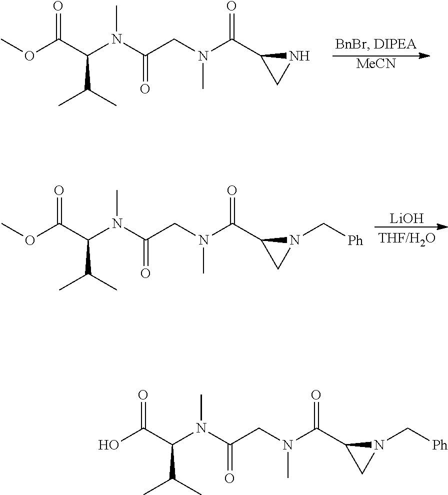

### Step 1: Synthesis of methyl N—(N—((S)-1-benzylaziridine-2-carbonyl)-N-methylglycyl)-N-methyl-L-valinate

To a solution of methyl N—(N—((S)-aziridine-2-carbonyl)-N-methylglycyl)-N-methyl-L-valinate (362.0 mg, 1.269 mmol) in MeCN (6.0 mL) at 0° C. was added DIPEA (883 μL, 5.08 mmol) and benzyl bromide (165 μL, 1.39 mmol). The reaction mixture was then warmed to room temperature and stirred overnight. The reaction mixture was then concentrated under reduced pressure. The residue was purified by prep-TLC (7% MeOH/DCM) to afford the desired product (287 mg, 60% yield) as an oil. LCMS (ESI) m/z: [M+H] calcd for C₂₀H₂₉N₃O₄: 376.22; found 376.2.

### Step 2: Synthesis of N—(N—((S)-1-benzylaziridine-2-carbonyl)-N-methylglycyl)-N-methyl-L-valine

To a solution of methyl N—(N—((S)-1-benzylaziridine-2-carbonyl)-N-methylglycyl)-N-methyl-L-valinate (270.0 mg, 0.719 mmol) in THF (3.6 mL) was added a solution of LiOH (86.1 mg, 3.59 mmol) in H₂O (3.60 mL). The reaction mixture was stirred for 30 min. The reaction was then quenched with sat. aq. NH₄Cl. The resulting mixture was extracted with EtOAc (3×15 mL) and the combined organic layers were washed with brine, dried over Na₂SO₄, filtered, and concentrated under reduced pressure to afford the desired crude product (240 mg, 92% yield) as an oil. LCMS (ESI) m/z: [M+H] calcd for C₁₉H₂₇N₃O₄: 362.21; found 362.2.

### Intermediate A-11. Synthesis of N-methyl-N—(N-methyl-N—((R)-1-tritylaziridine-2-carbonyl)glycyl)-L-valine

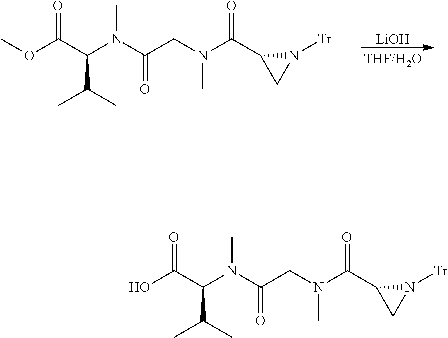

To a solution of methyl N-methyl-N—(N-methyl-N—((R)-1-tritylaziridine-2-carbonyl)glycyl)-L-valinate (1.30 g, 2.46 mmol) in THF (10.0 mL) at 0° C. was added a solution of LiOH (177.0 mg, 7.39 mmol) in H₂O (7.40 mL). The resulting mixture was warmed to room temperature, stirred for 3 h, and was then acidified to pH 5 with HCl (aq). The resulting mixture was extracted with EtOAc (3×80 mL) and the combined organic layers were washed with brine (2×50 mL), dried over Na₂SO₄, filtered, and concentrated under reduced pressure to afford the desired crude product (1 g, 71% yield). LCMS (ESI) m/z: [M+H] calcd for C₃₁H₃₅N₃O₄: 514.27; found 514.3.

### Intermediate A-12. Synthesis of N-methyl-N-(4-((R)-1-tritylaziridine-2-carbonyl)-1,4-diazepane-1-carbonyl)-L-valine

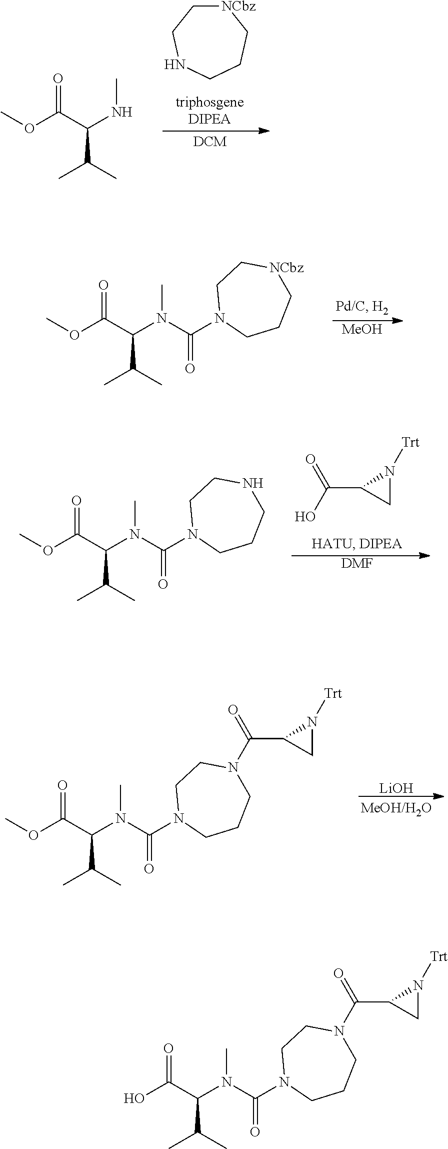

### Step 1: Synthesis of benzyl (S)-4-((1-methoxy-3-methyl-1-oxobutan-2-yl)(methyl)carbamoyl)-1,4-diazepane-1-carboxylate

To a solution of methyl N-methyl-L-valinate (2.50 g, 17.22 mmol) in DCM at 0° C. was added DIPEA (1.8 mL, 10.33 mmol) followed by triphosgene (2.55 g, 8.61 mmol). The resulting mixture was stirred for 3 h at 0° C. To the mixture was then added benzyl 1,4-diazepane-1-carboxylate (4.03 g, 17.20 mmol). The resulting mixture was warmed to room temperature and stirred overnight. The reaction was cooled to 0° C. and was quenched with NaHCO₃. The aqueous layer was extracted with EtOAc (2×30 mL) and the combined organic layers were washed with brine (2×30 mL), dried over Na₂SO₄, filtered, and concentrated under reduced pressure. The residue was purified by normal phase chromatography (25% EtOAc/pet. ether) to afford the desired product (3.5 g, 50.1% yield). LCMS (ESI) m/z: [M+H] calcd for C₂₁H₃₁N₃O₅: 406.23; found 406.5.

### Step 2: Synthesis of methyl N-(1,4-diazepane-1-carbonyl)-N-methyl-L-valinate

To a solution of benzyl (S)-4-((1-methoxy-3-methyl-1-oxobutan-2-yl)(methyl)carbamoyl)-1,4-diazepane-1-carboxylate (2.0 g, 4.93 mmol) in MeOH (20 mL) was added Pd/C (10% wt, 1 g). The mixture was placed under a hydrogen atmosphere (1 atm) and stirred for 2 h. The reaction mixture was filtered through a Celite and concentrated under reduced pressure to afford the desired crude product (1.3 g, 97.1% yield). LCMS (ESI) m/z: [M+H] calcd for C₁₃H₂₅N₃O₃: 272.20; found 272.3.

### Step 3: Synthesis of methyl N-methyl-N-(4-((R)-1-tritylaziridine-2-carbonyl)-1,4-diazepane-1-carbonyl)-L-valinate

To a solution of methyl N-(1,4-diazepane-1-carbonyl)-N-methyl-L-valinate (1.0 g, 3.69 mmol) and (R)-1-tritylaziridine-2-carboxylic acid (1.46 g, 4.42 mmol) in DMF at 0° C. was added DIPEA (1.93 mL, 11.06 mmol) followed by HATU (2.10 g, 5.52 mmol). The resulting mixture was warmed to room temperature and stirred for 1 h. The reaction mixture was then diluted with H₂O (15 mL) and the aqueous layer was extracted with EtOAc (3×30 mL). The combined organic layers were washed with brine (3×30 mL), dried over Na₂SO₄, filtered, and concentrated under reduced pressure. The residue was purified by normal phase chromatography (25% EtOAc/pet. ether) to afford the desired product (1.6 g, 74.5% yield). LCMS (ESI) m/z: [M+H] calcd for C₃₅H₄₂N₄O₄: 583.33; found 583.5.

### Step 4: Synthesis of N-methyl-N-(4-((R)-1-tritylaziridine-2-carbonyl)-1,4-diazepane-1-carbonyl)-L-valine

To a solution of methyl N-methyl-N-(4-((R)-1-tritylaziridine-2-carbonyl)-1,4-diazepane-1-carbonyl)-L-valinate (1.60 g, 2.75 mmol) in MeOH (10.0 mL) and H₂O (5.0 mL) at 0° C. was added LiOH (0.66 g, 27.56 mmol). The resulting mixture was warmed to room temperature and stirred overnight. The reaction mixture was acidified to pH 5 with HCl (aq) and the aqueous layer was extracted with EtOAc (3×30 mL). The combined organic layers were washed with brine (3×30 mL), dried over Na₂SO₄, filtered, concentrated under reduced pressure to afford the desired crude product (1.4 g, 95.6% yield). LCMS (ESI) m/z: [M+H] calcd for C₃₄H₄₀N₄O₄: 569.31; found 569.5.

### Intermediate A-13. Synthesis of N-methyl-N-(4-((S)-1-tritylaziridine-2-carbonyl)-1,4-diazepane-1-carbonyl)-L-valine

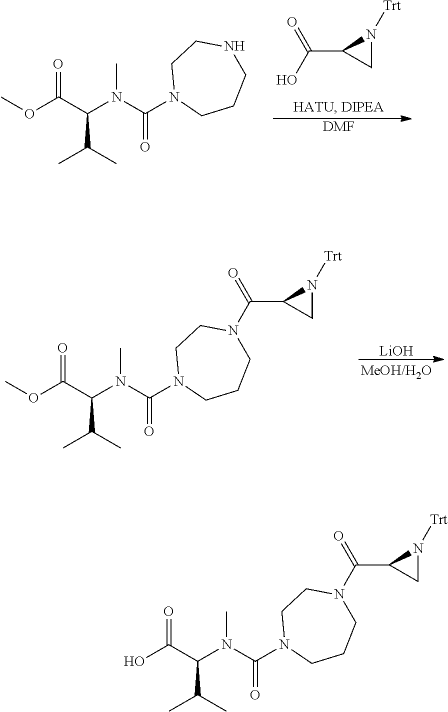

### Step 1: Synthesis of methyl N-methyl-N-(4-((S)-1-tritylaziridine-2-carbonyl)-1,4-diazepane-1-carbonyl)-L-valinate

To a solution of methyl N-(1,4-diazepane-1-carbonyl)-N-methyl-L-valinate (1.16 g, 4.28 mmol) and (S)-1-tritylaziridine-2-carboxylic acid (1.69 g, 5.13 mmol) in DMF (10 mL) at 0° C. was added DIPEA (2.23 mL, 12.82 mmol) followed by HATU (2.44 g, 6.41 mmol). The resulting mixture was stirred for 1 h at 0° C. The reaction mixture was then diluted with H₂O (15 mL) and the aqueous layer was extracted with EtOAc (3×15 mL). The combined organic layers were washed with brine (3×15 mL), dried over Na₂SO₄, filtered, and concentrated under reduced pressure. The residue was purified by normal phase chromatography (17% EtOAc/pet. ether) to afford the desired product (2 g, 80.3% yield). LCMS (ESI) m/z: [M+H] calcd for C₃₅H₄₂N₄O₄: 583.33; found 583.5.

### Step 2: Synthesis of N-methyl-N-(4-((S)-1-tritylaziridine-2-carbonyl)-1,4-diazepane-1-carbonyl)-L-valine

To a solution of methyl N-methyl-N-(4-((S)-1-tritylaziridine-2-carbonyl)-1,4-diazepane-1-carbonyl)-L-valinate (1.0 g, 1.72 mmol) in MeOH (8.0 mL) and H₂O (4.0 mL) at 0° C. was added LiOH (411 mg, 17.16 mmol). The resulting mixture was warmed to room temperature and stirred overnight. The reaction mixture was acidified to pH 5 with HCl (aq) and the aqueous layer was extracted with EtOAc (3×10 mL). The combined organic layers were washed with brine (3×10 mL), dried over Na₂SO₄, filtered, concentrated under reduced pressure to afford the desired crude product (0.6 g, 61.5% yield). LCMS (ESI) m/z: [M+H] calcd for C₃₄H₄₀N₄O₄: 569.31; found 569.5.

### Intermediate A-14. Synthesis of N-methyl-N-(5-((S)-1-tritylaziridine-2-carboxamido)picolinoyl)-L-valine

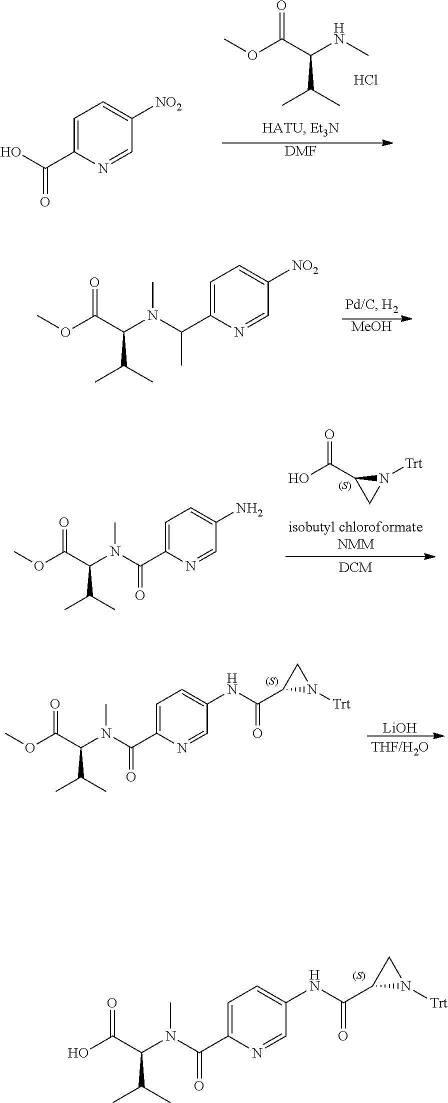

### Step 1: Synthesis of methyl N-methyl-N-(5-nitropicolinoyl)-L-valinate

To a solution of methyl N-methyl-L-valinate hydrochloride (190.0 mg, 1.31 mmol) and 5-nitropicolinic acid (200.0 mg, 1.19 mmol) in DMF (2 mL) at 0° C. was added HATU (678.6 mg, 1.79 mmol) and Et₃N (0.332 mL, 2.38 mmol). The resulting mixture was warmed to room temperature and stirred for 2 h. The resulting mixture was then extracted with EtOAc (2×50 mL) and the combined organic layers were washed with H₂O (20 mL) and brine (20 mL), dried over Na₂SO₄, filtered, and concentrated under reduced pressure. The residue was purified by normal phase chromatography (33% EtOAc/pet. ether) to afford the desired product (210 mg, 59.8% yield). LCMS (ESI) m/z: [M+H] calcd for C₁₃H₁₇N₃O₅: 296.12; found 296.0.

### Step 2: Synthesis of methyl N-(5-aminopicolinoyl)-N-methyl-L-valinate

To a solution of methyl N-methyl-N-(5-nitropicolinoyl)-L-valinate (5.0 g, 16.93 mmol) in MeOH (50.0 mL) was added Pd/C (2.50 g). The reaction mixture was placed under a hydrogen atmosphere (1 atm) and was stirred for 2 h. The mixture was filtered, the filter cake was washed with MeOH (2×20 mL), and the filtrate was concentrated under reduced pressure to afford the desired crude product (5.3 g). LCMS (ESI) m/z: [M+H] calcd for C₁₃H₁₉N₃O₃: 266.15; found 266.0.

### Step 3: Synthesis of methyl N-methyl-N-(5-((S)-1-tritylaziridine-2-carboxamido)picolinoyl)-L-valinate

To a solution (S)-1-tritylaziridine-2-carboxylic acid (55.9 mg, 0.17 mmol) in DCM at 0° C. was added isobutyl chloroformate (21.7 μL, 0.23 mmol) and N-methylmorpholine (66.8 μL, 0.61 mmol). The resulting mixture was stirred for 1 h and then methyl N-(5-aminopicolinoyl)-N-methyl-L-valinate (30.0 mg, 0.11 mmol) was added. The resulting mixture was warmed to room temperature and stirred for an additional 5 h. The mixture was extracted with DCM (3×50 mL) and the combined organic layers were washed with sat. NaHCO₃ (30 mL) and brine, dried over Na₂SO₄, filtered, and concentrated under reduced pressure. The residue was purified by normal phase chromatography (33% EtOAc/pet. ether) to afford the desired product (1.09 g, 66.9% yield). LCMS (ESI) m/z: [M+H] calcd for C₃₅H₃₆N₄O₄: 577.28; found 577.1.

### Step 4: Synthesis of N-methyl-N-(5-((S)-1-tritylaziridine-2-carboxamido)picolinoyl)-L-valine

To a solution methyl N-methyl-N-(5-((S)-1-tritylaziridine-2-carboxamido)picolinoyl)-L-valinate (100.0 mg, 0.17 mmol) in THF (0.5 mL) at 0° C. was added a solution of LiOH (20.76 mg, 0.87 mmol) in H₂O (0.5 mL). The resulting mixture was warmed to room temperature and stirred for 6 h. The mixture was acidified to pH 5 with 1 M citric acid. The resulting mixture was extracted with EtOAc (3×20 mL) and the combined organic layers were washed with brine (5 mL), dried over Na₂SO₄, filtered, and concentrated under reduced pressure to afford the desired crude product (76.8 mg, 78.7% yield). LCMS (ESI) m/z: [M+H] calcd for C₃₄H₃₄N₄O₄: 563.27; found 563.3.

### Intermediate A-15. Synthesis of N-methyl-N-(5-((R)-1-tritylaziridine-2-carboxamido)picolinoyl)-L-valine

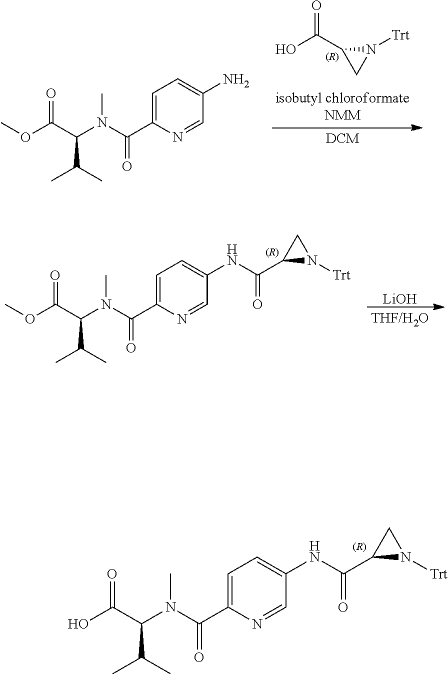

### Step 1: Synthesis of methyl N-methyl-N-(5-((R)-1-tritylaziridine-2-carboxamido)picolinoyl)-L-valinate

To a solution (R)-1-tritylaziridine-2-carboxylic acid (1396.7 mg, 4.24 mmol) in DCM (8 mL) at 0° C. was added isobutyl chloroformate (440 μL, 3.39 mmol) and N-methylmorpholine (466 μL, 4.24 mmol). The resulting mixture was stirred for 1 h and then methyl N-(5-aminopicolinoyl)-N-methyl-L-valinate (750.0 mg, 2.83 mmol) was added. The resulting mixture was warmed to room temperature and stirred for an additional 5 h. The mixture was quenched by the addition of NaHCO₃ and the aqueous layer was extracted with DCM (2×100 mL). The combined organic layers were washed with brine (120 mL), dried over Na₂SO₄, filtered, and concentrated under reduced pressure. The residue was purified by normal phase chromatography (50% EtOAc/pet. ether) to afford the desired product (580 mg, 35.6% yield). LCMS (ESI) m/z: [M+H] calcd for C₃₅H₃₆N₄O₄: 577.28; found 577.2.

### Step 2: Synthesis of N-methyl-N-(5-((R)-1-tritylaziridine-2-carboxamido)picolinoyl)-L-valine

To a solution methyl N-methyl-N-(5-((R)-1-tritylaziridine-2-carboxamido)picolinoyl)-L-valinate (558.0 mg, 0.97 mmol) in THF (14 mL) at 0° C. was added a solution of LiOH (115.9 mg, 4.84 mmol) in H₂O (14 mL). The resulting mixture was warmed to room temperature and stirred for 6 h. The mixture was acidified to pH 5 with 1 M citric acid. The resulting mixture was extracted with EtOAc (3×100 mL) and the combined organic layers were washed with brine (30 mL), dried over Na₂SO₄, filtered, and concentrated under reduced pressure to afford the desired crude product (580 mg, 78.7% yield). LCMS (ESI) m/z: [M+H] calcd for C₃₄H₃₄N₄O₄: 563.27; found 563.2.

### Intermediate A-16. Synthesis of (2R,3S)-1-((R)-tert-butylsulfinyl-3-(methoxycarbonyl)aziridine-2-carboxylic acid

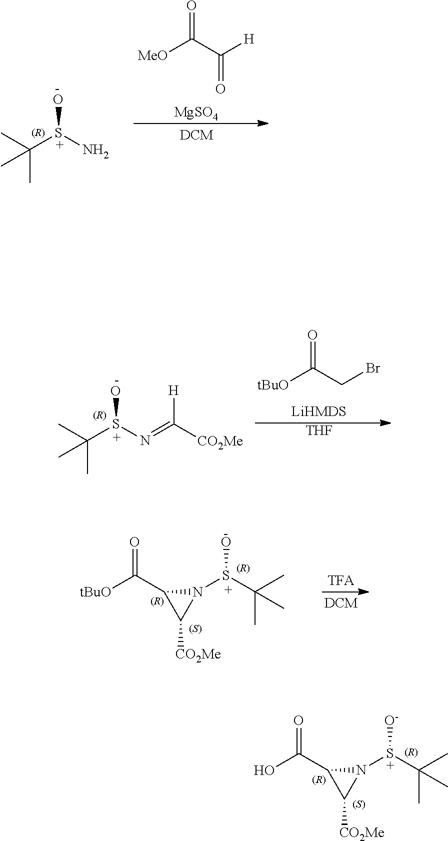

### Step 1: Synthesis of methyl (R,E)-2-((tert-butylsulfinyl)imino)acetate

To a solution of (R)-2-methylpropane-2-sulfinamide (13.21 g, 109.01 mmol) and methyl 2-oxoacetate (8.0 g, 90.85 mmol) in DCM (130 mL) at room temperature was added MgSO₄ (54.67 g, 454.23 mmol). The resulting mixture was heated to 35° C. and stirred for 16 h. The resulting mixture was filtered, the filter cake washed with EtOAc (3×50 mL), and the filtrate was concentrated under reduced pressure. The residue was purified by normal phase chromatography (25% EtOAc/pet. ether) to afford the desired (5.8 g, 33.4% yield). LCMS (ESI) m/z: [M+H] calcd for C₇H₁₃NO₃S: 192.07; found 191.9.

### Step 2: Synthesis of 2-(tert-butyl) 3-methyl (2R,3S)-1-((R)-tert-butylsulfinyl)aziridine-2,3-dicarboxylate

To a solution of 1M LiHMDS (61.40 mL, 61.40 mmol) in THF (300.0 mL) at −78° C. was added tert-butyl 2-bromoacetate (11.83 g, 60.65 mmol). The resulting mixture was stirred for 30 min. To the reaction mixture was then added methyl methyl (R,E)-2-((tert-butylsulfinyl)imino)acetate (5.8 g, 30.33 mmol). The resulting mixture was warmed to −60° C. and stirred for 2.5 h. The reaction was warmed to 0° C. and quenched with sat. NH₄Cl (aq.). The resulting mixture was extracted with EtOAc (3×200 mL). The combined organic layers were washed with brine (500 mL), dried over Na₂SO₄, filtered, and concentrated under reduced pressure. The residue was purified by reverse phase chromatography (10→50% MeCN/H₂O) to afford the desired product (1.34 g, 4.5% yield). LCMS (ESI) m/z: [M+H] calcd for C₁₃H₂₃NO₅S: 306.14; found 306.2.

### Step 3: Synthesis of (2R,3S)-1-((R)-tert-butylsulfinyl)-3-(methoxycarbonyl)aziridine-2-carboxylic acid

To a solution of 2-(tert-butyl) 3-methyl (2R,3S)-1-((R)-tert-butylsulfinyl)aziridine-2,3-dicarboxylate (302.0 mg, 0.99 mmol) in DCM (3.0 mL) at 0° C. was added TFA (1.50 mL). The resulting mixture was stirred for 1 h and then concentrated under reduced pressure to afford the desired crude product (300 mg). LCMS (ESI) m/z: [M+H] calcd for C₉H₁₅NO₅S: 250.07; found 250.1.

### Intermediate A-17. Synthesis of (2R,3S)-1-((S)-tert-butylsulfinyl)-3-(methoxycarbonyl)aziridine-2-carboxylic acid

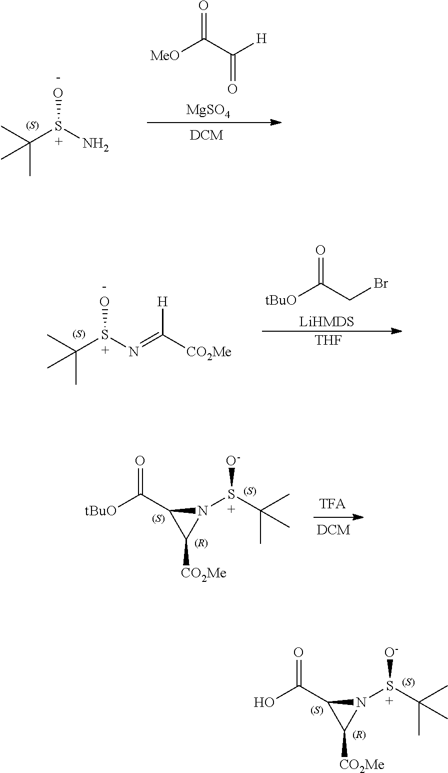

### Step 1: Synthesis of methyl (S,E)-2-((tert-butylsulfinyl)imino)acetate

To a solution of (S)-2-methylpropane-2-sulfinamide (9.81 g, 80.94 mmol) and methyl 2-oxoacetate (5.94 g, 67.45 mmol) in DCM (100 mL) at room temperature was added MgSO₄ (40.60 g, 337.26 mmol). The resulting mixture was heated to 35° C. and stirred for 16 h. The resulting mixture was filtered, the filter cake washed with EtOAc (3×50 mL), and the filtrate was concentrated under reduced pressure. The residue was purified by normal phase chromatography (25% EtOAc/pet. ether) to afford the desired (5.68 g, 44.0% yield). LCMS (ESI) m/z: [M+H] calcd for C₇H₁₃NO₃S: 192.07; found 191.1.

### Step 2: Synthesis of 2-(tert-butyl) 3-methyl (2R,3S)-1-((S)-tert-butylsulfinyl)aziridine-2,3-dicarboxylate

To a solution of 1M LiHMDS (59.40 mL, 59.40 mmol) in THF (300.0 mL) at −78° C. was added tert-butyl 2-bromoacetate (11.59 g, 59.40 mmol). The resulting mixture was stirred for 30 min. To the reaction mixture was then added methyl methyl (S,E)-2-((tert-butylsulfinyl)imino)acetate (5.68 g, 29.70 mmol). The resulting mixture was warmed to −60° C. and stirred for 2.5 h. The reaction was warmed to 0° C. and quenched with sat. NH₄Cl (aq.). The resulting mixture was extracted with EtOAc (3×200 mL). The combined organic layers were washed with brine (500 mL), dried over Na₂SO₄, filtered, and concentrated under reduced pressure. The residue was purified by reverse phase chromatography (10→50% MeCN/H₂O) to afford the desired product (1.26 g, 13.9% yield). LCMS (ESI) m/z: [M+H] calcd for C₁₃H₂₃NO₅S: 306.14; found 306.1.

### Step 3: Synthesis of (2R,3S)-1-((S)-tert-butylsulfinyl)-3-(methoxycarbonyl)aziridine-2-carboxylic acid

To a solution of 2-(tert-butyl) 3-methyl (2R,3S)-1-((S)-tert-butylsulfinyl)aziridine-2,3-dicarboxylate (457.0 mg, 1.50 mmol) in DCM (6.0 mL) at 0° C. was added TFA (3.0 mL). The resulting mixture was stirred for 1 h and then concentrated under reduced pressure to afford the desired crude product (450 mg). LCMS (ESI) m/z: [M+H] calcd for C₉H₁₅NO₅S: 250.07; found 250.1.

### Intermediate A-18. Synthesis of (2R,3R)-1-((R)-tert-butylsulfinyl)-3-cyclopropylaziridine-2-carboxylic acid

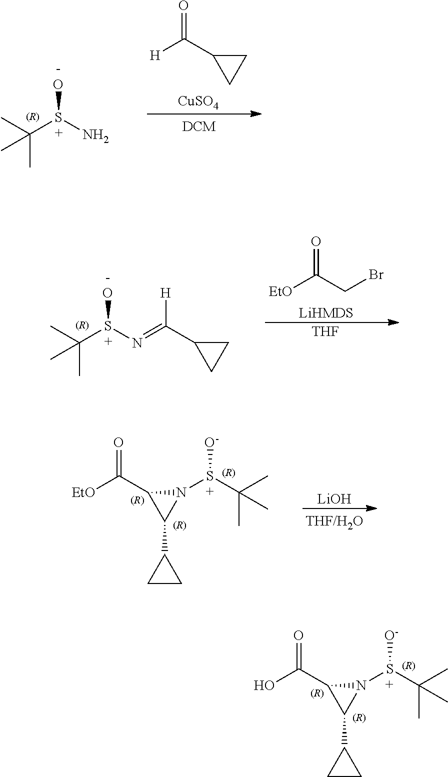

### Step 1: Synthesis of (R,E)-N-(cyclopropylmethylene)-2-methylpropane-2-sulfinamide

To a solution of (R)-2-methylpropane-2-sulfinamide (1.0 g, 8.25 mmol) and cyclopropanecarbaldehyde (1.16 g, 16.55 mmol) in DCM (50 mL) at room temperature was added CuSO₄ (3.95 g, 24.75 mmol). The resulting mixture was stirred overnight. The reaction mixture was then filtered, the filter cake washed with EtOAc, and the filtrate was concentrated under reduced pressure. The residue was purified by prep-TLC (17% EtOAc/pet. ether) to afford the desired product (1.4 g, 97.9% yield). LCMS (ESI) m/z: [M+H] calcd for C₈H₁₅NOS: 174.10; found 174.1.

### Step 2: Synthesis of ethyl (2R,3R)-1-((R)-tert-butylsulfinyl)-3-cyclopropylaziridine-2-carboxylate

To a solution of 1M LiHMDS (23 mL, 23 mmol) in THF (50.0 mL) at −78° C. was added ethyl bromoacetate (3.83 g, 22.95 mmol). The resulting mixture was warmed to −70° C. and stirred for 1 h. To the reaction mixture was then added (R,E)-N-(cyclopropylmethylene)-2-methylpropane-2-sulfinamide (2.0 g, 11.48 mmol). The resulting mixture was stirred for 1 h at −70° C. The reaction mixture was warmed to 0° C. and quenched with H₂O. The aqueous layer was extracted with EtOAc (3×100 mL). The combined organic layers were washed with brine, dried over Na₂SO₄, filtered, and concentrated under reduced pressure. The residue was purified by prep-TLC (25% EtOAc/pet. ether) to afford the desired product (1.8 g, 60.5% yield). LCMS (ESI) m/z: [M+H] calcd for C₁₂H₂₁NO₃S: 306.14; found 260.13.

### Step 3: Synthesis of (2R,3R)-1-((R)-tert-butylsulfinyl)-3-cyclopropylaziridine-2-carboxylic acid

To a solution of ethyl (2R,3R)-1-((R)-tert-butylsulfinyl)-3-cyclopropylaziridine-2-carboxylate (900.0 mg, 3.47 mmol) in THF (3.0 mL) and H₂O (3.0 mL) at 0° C. was added LiOH·H₂O (218.4 mg, 5.21 mmol). The resulting mixture was stirred for 1 h and was then quenched by H₂O. The aqueous layer was extracted with EtOAc (3×50) and the combined organic layers were washed with brine, dried over Na₂SO₄, filtered, and concentrated under reduced pressure to afford the desired crude product (400 mg, 29.9% yield). LCMS (ESI) m/z: [M+H] calcd for C₁₀H₁₇NO₃S: 232.10; found 232.1.

### Intermediate A-19. Synthesis of (2R,3R)-1-((R)-tert-butylsulfinyl)-3-methylaziridine-2-carboxylic acid

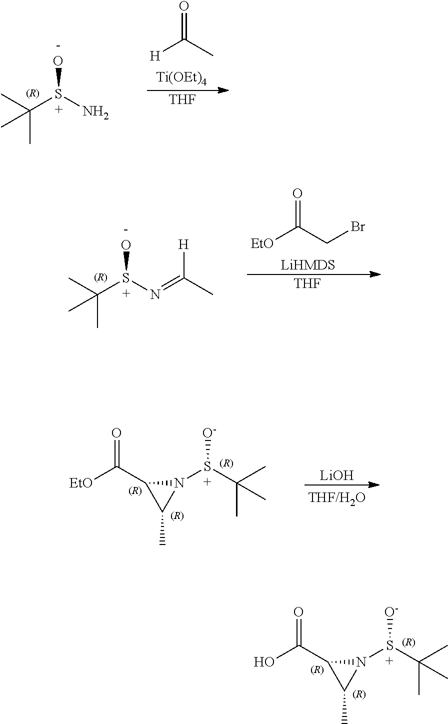

### Step 1: Synthesis of (R,E)-N-ethylidene-2-methylpropane-2-sulfinamide

To a solution of (R)-2-methylpropane-2-sulfinamide (3.0 g, 24.75 mmol) and tetraethoxytitanium (1.7 g, 7.43 mmol) in THF (30 mL) at 0° C. was added acetaldehyde (218.1 mg, 4.95 mmol). The resulting mixture was stirred for 20 min and was then quenched with H₂O (100 mL). The suspension was filtered, and the filter cake washed with EtOAc (3×100 mL). The aqueous layer was extracted with EtOAc (3×100 mL) and the combined organic layers were washed with brine (3×100 mL), dried over Na₂SO₄, filtered, and concentrated under reduced pressure. Purification by normal phase chromatography (9% EtOAc/pet. ether) afforded desired product (3 g, 82% yield). LCMS (ESI) m/z: [M+H] calcd for C₆H₁₃NOS: 148.08; found 148.0.

### Step 2: Synthesis of ethyl (2R,3R)-1-((R)-tert-butylsulfinyl)-3-methylaziridine-2-carboxylate

To a solution of 1M LiHMDS (40.75 mL, 40.75 mmol) in THF (30.0 mL) at −78° C. was added ethyl bromoacetate (6.80 g, 40.75 mmol). The resulting mixture was stirred for 1 h. To the reaction mixture was then added (R,E)-N-ethylidene-2-methylpropane-2-sulfinamide (3.0 g, 20.38 mmol). The resulting mixture was stirred for 2 h at −78° C. and then quenched with H₂O (300 mL). The aqueous layer was extracted with EtOAc (3×300 mL) and the combined organic layers were washed with brine (3×100 mL), dried over Na₂SO₄, filtered, and concentrated under reduced pressure. The residue was purified by reverse phase chromatography (10→50% MeCN/H₂O) to afford the desired product (1.4 g, 29.5% yield). LCMS (ESI) m/z: [M+H] calcd for C₁₀H₁₉NO₃S: 234.12; found 234.1.

### Step 3: Synthesis of (2R,3R)-1-((R)-tert-butylsulfinyl)-3-methylaziridine-2-carboxylic acid

To a solution of ethyl (2R,3R)-1-((R)-tert-butylsulfinyl)-3-methylaziridine-2-carboxylate (1.0 g, 4.29 mmol) in THF (6.4 mL) and H₂O (6.4 mL) at 0° C. was added LiOH·H₂O (539.5 mg, 12.86 mmol). The resulting mixture was warmed to room temperature and stirred for 2 h and was then neutralized to pH 5 with HCl (aq.) and sat. NH₄Cl (aq.). The aqueous layer was extracted with EtOAc (3×10 mL) and the combined organic layers were washed with brine, dried over Na₂SO₄, filtered, and concentrated under reduced pressure to afford the desired crude product (489 mg, 55.6% yield). LCMS (ESI) m/z: [M+H] calcd for C₈H₁₅NO₃S: 206.09; found 206.0.

### Intermediate A-20. Synthesis of (2S,3S)-1-(S)-tert-butylsulfinyl)-3-methylaziridine-2-carboxylic acid

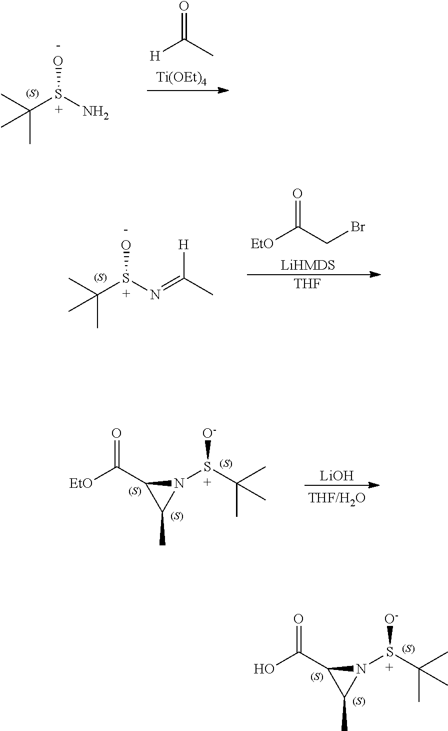

### Step 1: Synthesis of (S,E)-N-ethylidene-2-methylpropane-2-sulfinamide

To a mixture of (S)-2-methylpropane-2-sulfinamide (5.0 g, 41.25 mmol) and tetraethoxytitanium (18.82 g, 82.51 mmol) at 0° C. was added acetaldehyde (3.63 g, 82.51 mmol). The resulting mixture was warmed to room temperature and stirred for 30 min and was then quenched with H₂O (100 mL). The suspension was filtered, and the filter cake washed with EtOAc (3×100 mL). The aqueous layer was extracted with EtOAc (3×100 mL) and the combined organic layers were washed with brine (3×100 mL), dried over Na₂SO₄, filtered, and concentrated under reduced pressure to afford desired crude product (3.9 g, 64% yield). LCMS (ESI) m/z: [M+H] calcd for C₆H₁₃NOS: 148.08; found 148.2.

### Step 2: Synthesis of ethyl (2S,3S)-1-((S)-tert-butylsulfinyl)-3-methylaziridine-2-carboxylate

To a solution of 1M LiHMDS (40.75 mL, 40.75 mmol) in THF (30.0 mL) at −78° C. was added ethyl bromoacetate (6.80 g, 40.75 mmol). The resulting mixture was stirred for 1 h. To the reaction mixture was then added (S,E)-N-ethylidene-2-methylpropane-2-sulfinamide (3.0 g, 20.38 mmol). The resulting mixture was stirred for 2 h at −78° C. and then quenched with H₂O. The aqueous layer was extracted with EtOAc (3×200 mL) and the combined organic layers were washed with brine (3×300 mL), dried over Na₂SO₄, filtered, and concentrated under reduced pressure. The residue was purified by reverse phase chromatography (10→50% MeCN/H₂O) to afford the desired product (2 g, 42% yield). LCMS (ESI) m/z: [M+H] calcd for C₁₀H₁₉NO₃S: 234.12; found 234.0.

### Step 3: Synthesis of (2S,3S)-1-((S)-tert-butylsulfinyl)-3-methylaziridine-2-carboxylic acid

To a solution of ethyl (2S,3S)-1-((S)-tert-butylsulfinyl)-3-methylaziridine-2-carboxylate (80.0 mg, 0.34 mmol) in THF (1.0 mL) and H₂O (0.2 mL) at 0° C. was added LiOH·H₂O (32.9 mg, 1.37 mmol). The resulting mixture was warmed to room temperature and stirred for 4 h and was then acidified to pH 3 with HCl (aq.). The aqueous layer was extracted with EtOAc (3×10 mL) and the combined organic layers were washed with brine, dried over Na₂SO₄, filtered, and concentrated under reduced pressure to afford the desired crude product (70 mg, 99% yield). LCMS (ESI) m/z: [M+H] calcd for C₈H₁₅NO₃S: 206.09; found 206.0.

### Intermediate A-21 and A-22. Synthesis of N-methyl-N—((S)-1-(((R)-1-methylaziridin-2-yl)sulfonyl)pyrrolidine-3-carbonyl)-L-valine and N-methyl-N—((S)-1-(((S)-1-methylaziridin-2-yl)sulfonyl)pyrrolidine-3-carbonyl)-L-valine

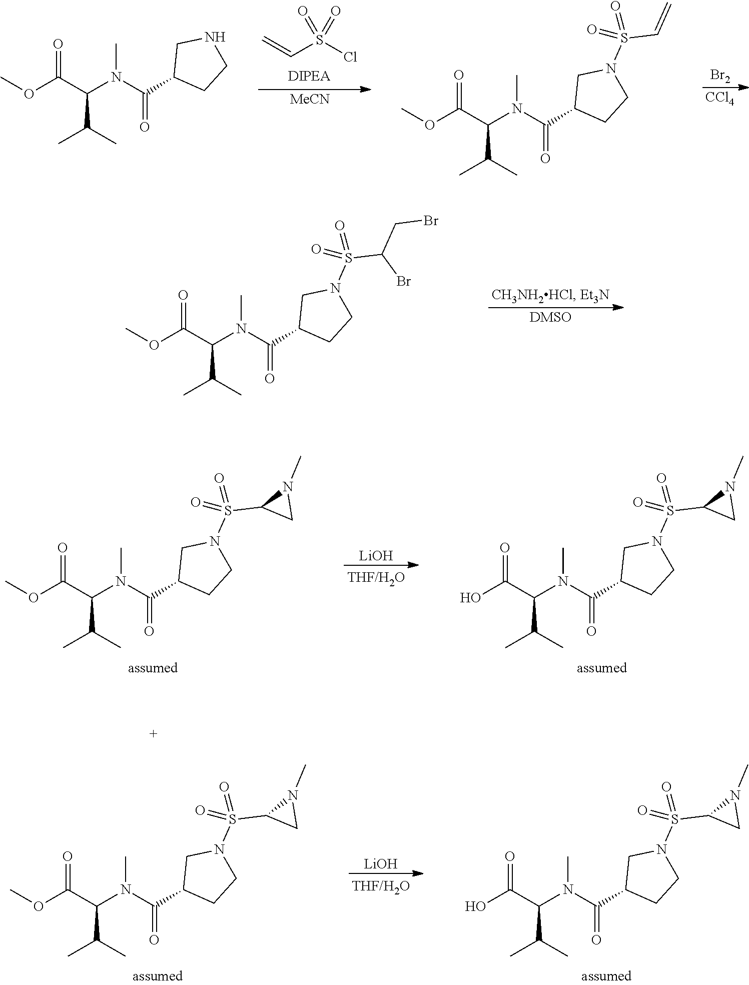

### Step 1: Synthesis of methyl N-methyl-N—((S)-1-(vinylsulfonyl)pyrrolidine-3-carbonyl)-L-valinate

To a mixture of methyl N-methyl-N—((S)-pyrrolidine-3-carbonyl)-L-valinate (7.0 g, 28.89 mmol) in MeCN (200 mL) at −20° C. was added DIPEA (10.0 mL, 57.78 mmol) followed by ethenesulfonyl chloride (4.0 g, 31.78 mmol). The resulting solution was stirred for 2 h at −20° C. and was then diluted with EtOAc (800 mL). The resulting solution was washed with brine (3×100 mL) and concentrated under reduced pressure. Purification by normal phase chromatography (50% EtOAc/pet. ether) afforded the desired product (4.8 g, 49.9%, yield). LCMS (ESI) m/z: [M+H] calcd for C₁₄H₂₄N₂O₅S: 333.15; found 333.1.

### Step 2: Synthesis of methyl N-((3S)-1-((1,2-dibromoethyl)sulfonyl)pyrrolidine-3-carbonyl)-N-methyl-L-valinate

To a solution of methyl N-methyl-N—((S)-1-(vinylsulfonyl)pyrrolidine-3-carbonyl)-L-valinate (4.5 g, 13.54 mmol) in CCl₄ (100 mL) at 0° C. was added Br₂ (2.77 mL, 54.15 mmol). The resulting solution was stirred for overnight and was then quenched by the addition of sat. NaHCO₃ (100 mL). The aqueous layer was extracted with EtOAc (3×200 mL) and the combined organic layers were washed with brine, dried with Na₂SO₄, filtered, and concentrated under reduced pressure. Purification by normal phase chromatography (25% EtOAc/pet. ether) afforded the desired product (2.6 g, 39.0% yield). LCMS (ESI) m/z: [M+H] calcd for C₁₄H₂₄Br₂N₂O₅S: 492.99; found 493.0.

### Step 3: Synthesis of methyl N-methyl-N—((S)-1-(((R)-1-methylaziridin-2-yl)sulfonyl)pyrrolidine-3-carbonyl)-L-valinate and methyl N-methyl-N—((S)-1-(((S)-1-methylaziridin-2-yl)sulfonyl)pyrrolidine-3-carbonyl)-L-valinate

To a solution of methyl N-((3S)-1-((1,2-dibromoethyl)sulfonyl)pyrrolidine-3-carbonyl)-N-methyl-L-valinate (2.6 g, 5.28 mmol) in DMSO (250 mL) was added methanamine hydrochloride (1.07 g, 15.85 mmol) and Et₃N (7.37 mL, 52.82 mmol). The reaction mixture was heated to 75° C. and stirred overnight. The mixture was then cooled to room temperature and diluted with EtOAc (1.5 L). The resulting mixture was washed with sat. NH₄Cl (2×200 mL) and brine (2×200 mL) and the organic layer was then concentrated under reduced pressure. Purification by reverse phase chromatography (40→60% MeCN/H₂O) afforded a mixture of the desired products. The diastereomers were separated by prep-SFC (28% MeOH/CO₂) to afford methyl N-methyl-N—((S)-1-(((R)-1-methylaziridin-2-yl)sulfonyl)pyrrolidine-3-carbonyl)-L-valinate (0.46 g, 24% yield) and methyl N-methyl-N—((S)-1-(((S)-1-methylaziridin-2-yl)sulfonyl)pyrrolidine-3-carbonyl)-L-valinate (0.35 g, 18.3% yield). LCMS (ESI) m/z: [M+H] calcd for C₁₅H₂₇N₃O₅S: 362.17; found 362.1.

### Step 4: Synthesis of N-methyl-N—((S)-1-(((R)-1-methylaziridin-2-yl)sulfonyl)pyrrolidine-3-carbonyl)-L-valine

To a solution of methyl N-methyl-N—((S)-1-(((R)-1-methylaziridin-2-yl)sulfonyl)pyrrolidine-3-carbonyl)-L-valinate (200.0 mg, 0.55 mmol) in THF (2.0 mL) and H₂O (2.0 mL) at 0° C. was added LiOH (53.0 mg, 2.21 mmol). The resulting solution was stirred for 2 h at 0° C. and then the reaction mixture was acidified to pH 6 with 1M HCl. The aqueous layer was extracted with EtOAc and the combined organic layers were washed with brine, dried with Na₂SO₄, filtered, and concentrated under reduced pressure. Purification by reverse phase chromatography (5→55% MeCN/H₂O) afforded the desired product (110 mg, 57.2%, yield). LCMS (ESI) m/z: [M+H] calcd for C₁₄H₂₅N₃O₅S: 348.16; found 348.1.

### Step 5: Synthesis of N-methyl-N—((S)-1-(((S)-1-methylaziridin-2-yl)sulfonyl)pyrrolidine-3-carbonyl)-L-valine

To a solution of methyl N-methyl-N—((S)-1-(((S)-1-methylaziridin-2-yl)sulfonyl)pyrrolidine-3-carbonyl)-L-valinate (200.0 mg, 0.55 mmol) in THF (2.0 mL) and H₂O (2.0 mL) at 0° C. was added LiOH (53.0 mg, 2.21 mmol). The resulting solution was stirred for 2 h at 0° C. and then the reaction mixture was acidified to pH 6 with 1M HCl. The aqueous layer was extracted with EtOAc and the combined organic layers were washed with brine, dried with Na₂SO₄, filtered, and concentrated under reduced pressure. Purification by reverse phase chromatography (5→55 MeCN/H₂O) afforded the desired product (121 mg, 62.9%, yield). LCMS (ESI) m/z: [M+H] calcd for C₁₄H₂₅N₃O₅S: 348.16; found 348.1.

### Intermediate A-23. Synthesis of (2S)-3-methyl-2-(1-oxo-7-((S)-1-tritylaziridine-2-carbonyl)-2,7-diazaspiro[4.4]nonan-2-yl)butanoic acid

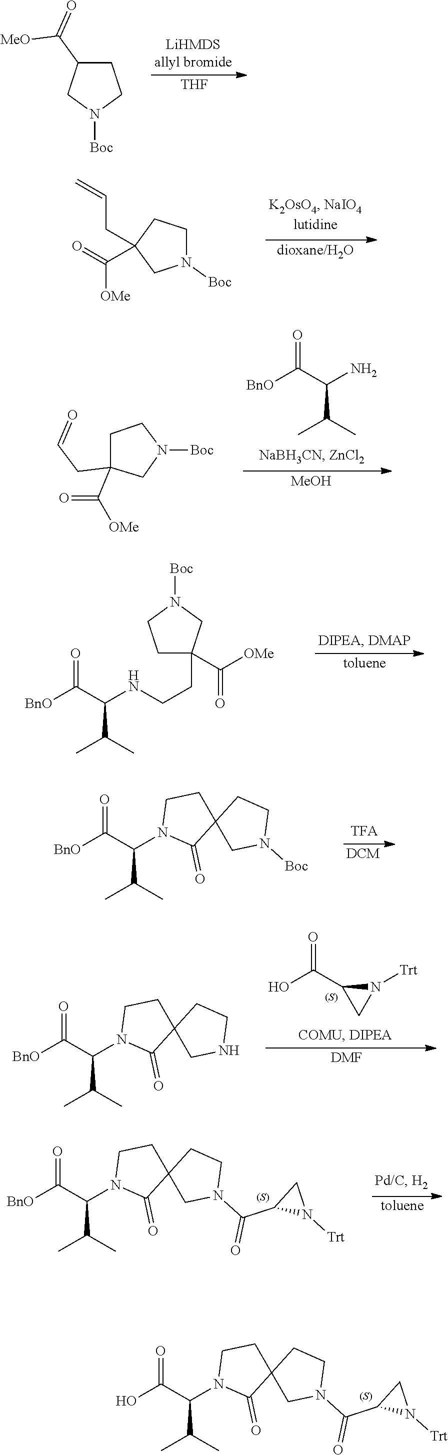

### Step 1: Synthesis of 1-(tert-butyl) 3-methyl 3-allylpyrrolidine-1,3-dicarboxylate

To a mixture of 1-(tert-butyl) 3-methyl pyrrolidine-1,3-dicarboxylate (10 g, 43.616 mmol) in THF (100 mL) at −78° C. was added 1 M LiHMDS (65.42 mL, 65.424 mmol) dropwise. The resulting mixture was stirred at −78° C. for 1 h and then a solution of allyl bromide (7.91 g, 65.423 mmol) in THF was added dropwise over 10 min. The resulting mixture was stirred at −78° C. for an additional 2 h and was then quenched by the addition of sat. NH₄Cl at 0° C. The resulting mixture was extracted with EtOAc (3×100 mL) and the combined organic layers were washed with brine (2×80 mL), dried with Na₂SO₄, filtered, and concentrated under reduced pressure. Purification by silica gel column chromatography (20% EtOAc/pet. ether) afforded the desired product (10 g, 76% yield).

### Step 2: Synthesis of 1-(tert-butyl) 3-methyl 3-(2-oxoethyl)pyrrolidine-1,3-dicarboxylate

To a mixture of 1-(tert-butyl) 3-methyl 3-allylpyrrolidine-1,3-dicarboxylate (11.0 g, 40.84 mmol) and 2,6-lutidine (8.75 g, 81.68 mmol) in dioxane (190 mL) and H₂O (19 mL) at 0° C. was added K₂OsO₄·2H₂O (0.75 g, 2.04 mmol). The resulting mixture was stirred at 0° C. for 15 min and then NaIO₄ (34.94 g, 163.36 mmol) was added in portions. The mixture was warmed to room temperature and stirred for an additional 3 h, then was quenched by the addition of sat. Na₂S2O₃ at 0° C. The resulting mixture was extracted with EtOAc (3×300 mL) and the combined organic layers were washed with brine (200 mL), dried with Na₂SO₄, filtered, and concentrated under reduced pressure. Purification by reverse phase chromatography (0→40% MeCN/H₂O, 0.1% HCO₂H) afforded the desired product (6.4 g, 51% yield).

### Step 3: Synthesis of 1-(tert-butyl) 3-methyl 3-(2-(((S)-1-(benzyloxy)-3-methyl-1-oxobutan-2-yl)amino)ethyl)pyrrolidine-1,3-dicarboxylate

To a mixture of 1-(tert-butyl) 3-methyl 3-(2-oxoethyl)pyrrolidine-1,3-dicarboxylate (6.30 g, 23.220 mmol) and benzyl L-valinate (7.22 g, 34.831 mmol) in MeOH (70 mL) at 0° C. was added ZnCl₂ (4.75 g, 34.831 mmol). The resulting mixture was warmed to room temperature and stirred for 30 min, then cooled to 0° C. NaBH₃CN (2.92 g, 46.441 mmol) was added in portions then the mixture was warmed to room temperature and stirred for 2 h. The reaction was quenched by the addition of sat. NH₄C₁ at 0° C. and the resulting mixture was then extracted with EtOAc (3×200 mL). The combined organic layers were washed with brine (150 mL), dried with Na₂SO₄, filtered, and concentrated under reduced pressure. Purification by silica gel column chromatography (33% EtOAc/pet. ether) afforded the desired product (6.4 g, 53% yield). LCMS (ESI) m/z: [M+H] calcd for C₂₅H₃₈N₂O₆: 463.28; found 463.3.

### Step 4: Synthesis of tert-butyl 7-((S)-1-(benzyloxy)-3-methyl-1-oxobutan-2-yl)-6-oxo-2,7-diazaspiro[4.4]nonane-2-carboxylate

To a mixture of 1-(tert-butyl) 3-methyl 3-(2-(((S)-1-(benzyloxy)-3-methyl-1-oxobutan-2-yl)amino)ethyl)pyrrolidine-1,3-dicarboxylate (4.50 g, 9.728 mmol) and DIPEA (16.6 mL, 97.28 mmol) in toluene (50 mL) was added DMAP (1.19 g, 9.728 mmol) and then mixture was heated to 80° C. After 24 h the reaction was cooled to room temperature and concentrated under reduced pressure. Purification by reverse phase chromatography (15→60% MeCN/H₂O, 0.1% HCO₂H) afforded the desired product (3 g, 64% yield). LCMS (ESI) m/z: [M+H] calcd for C₂₄H₃₄N₂O₅: 431.26; found 431.2.

### Step 5: Synthesis of benzyl (2S)-3-methyl-2-(1-oxo-2,7-diazaspiro[4.4]nonan-2-yl)butanoate

To a solution of tert-butyl 7-((S)-1-(benzyloxy)-3-methyl-1-oxobutan-2-yl)-6-oxo-2,7-diazaspiro[4.4]nonane-2-carboxylate (400.0 mg, 0.929 mmol) in DCM (3.0 mL) at 0° C. was added TFA (1.50 mL, 20.195 mmol) dropwise. The resulting mixture was stirred at 0° C. for 1 h and was then concentrated under reduced pressure. The TFA residue was further removed by azeotropic distillation with toluene three times to afford the desired product (400 mg, crude). LCMS (ESI) m/z: [M+H] calcd for C₁₉H₂₆N₂O₃: 331.20; found 331.1.

### Step 6: Synthesis of benzyl (2S)-3-methyl-2-(1-oxo-7-((S)-1-tritylaziridine-2-carbonyl)-2,7-diazaspiro[4.4]nonan-2-yl)butanoate

To a solution of benzyl (2S)-3-methyl-2-(1-oxo-2,7-diazaspiro[4.4]nonan-2-yl)butanoate (400.0 mg, 1.21 mmol) and DIPEA (2.06 mL, 12.11 mmol) in DMF (5 mL) at 0° C. was added (S)-1-tritylaziridine-2-carboxylic acid (558.26 mg, 1.695 mmol) followed by COMU (673.55 mg, 1.574 mmol) in portions. The resulting mixture was stirred at 0° C. for 1 h and was then diluted with H₂O (50 mL). The aqueous layer was extracted with EtOAc (3×20 mL) and the combined organic layers were washed with brine (20 mL), dried with Na₂SO₄, filtered, and concentrated under reduced pressure. Purification by prep-TLC (33% EtOAc/pet. ether) afforded the desired product (510 mg, 59% yield). LCMS (ESI) m/z: [M+H] calcd for C₄₁H₄₃N₃O₄: 642.34; found 642.3.

### Step 7: Synthesis of (2S)-3-methyl-2-(1-oxo-7-((S)-1-tritylaziridine-2-carbonyl)-2,7-diazaspiro[4.4]nonan-2-yl)butanoic acid

To a mixture of benzyl (2S)-3-methyl-2-(1-oxo-7-((S)-1-tritylaziridine-2-carbonyl)-2,7-diazaspiro[4.4]nonan-2-yl)butanoate (480.0 mg, 0.748 mmol) in toluene (35.0 mL) was added Pd/C 200.0 mg, 1.879 mmol). The resulting mixture was placed under an atmosphere of H₂ (1 atm), heated to 50° C. and stirred for 3 h. The mixture was cooled to room temperature, filtered, the filter cake was washed with MeOH (3×10 mL), and the filtrate was concentrated under reduced pressure to afford the desired product (310 mg, 67% yield). LCMS (ESI) m/z: [M−H] calcd for C₃₄H₃₇N₃O₄: 550.27; found 550.3.

### Intermediate A-24. Synthesis of (2S)-3-methyl-2-(1-oxo-7-((R)-1-tritylaziridine-2-carbonyl)-2,7-diazaspiro[4.4]nonan-2-yl)butanoic acid

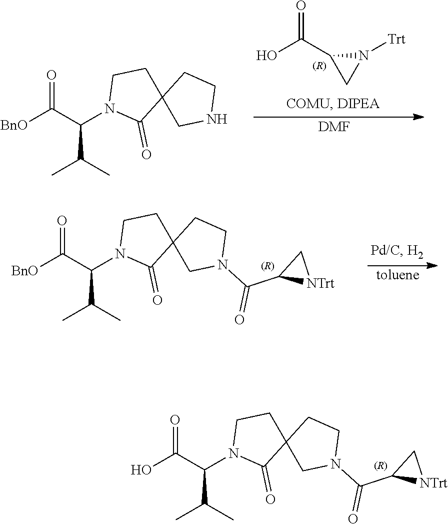

### Step 1: Synthesis of benzyl (2S)-3-methyl-2-(1-oxo-7-((R)-1-tritylaziridine-2-carbonyl)-2,7-diazaspiro[4.4]nonan-2-yl)butanoate

To a solution of benzyl (2S)-3-methyl-2-(1-oxo-2,7-diazaspiro[4.4]nonan-2-yl)butanoate (400.0 mg, 1.21 mmol) and (R)-1-tritylaziridine-2-carboxylic acid (518.4 mg, 1.57 mmol) in DMF (4.0 mL) at 0° C. was added DIPEA (1.0 mL, 6.05 mmol) followed by COMU (621.7 mg, 1.45 mmol). The resulting mixture was stirred for 1 h and was then diluted with H₂O (40 mL). The aqueous layer was extracted with EtOAc (3×15 mL) and the combined organic layers were washed with brine (2×10 mL), dried over Na₂SO₄, filtered and concentrated under reduced pressure. The residue was purified by prep-TLC (33% EtOAc/pet. ether) to afford the desired product (540 mg, 62% yield). LCMS (ESI) m/z: [M+H] calcd for C₄₁H₄₃N₃O₄: 642.33; found 642.4.

### Step 2: Synthesis of (2S)-3-methyl-2-(1-oxo-7-((R)-1-tritylaziridine-2-carbonyl)-2,7-diazaspiro[4.4]nonan-2-yl)butanoic acid

To a solution of benzyl (2S)-3-methyl-2-(1-oxo-7-((R)-1-tritylaziridine-2-carbonyl)-2,7-diazaspiro[4.4]nonan-2-yl)butanoate (510.0 mg, 0.80 mmol) in toluene (30 mL) was added Pd/C (250.0 mg, 2.35 mmol). The resulting mixture was placed under a hydrogen atmosphere (1 atm), heated to 50° C., and stirred for 3 h. The reaction was then cooled to room temperature, filtered, the filter cake was washed with MeOH (3×10 mL), and the filtrate was concentrated under reduced pressure to afford the desired crude product (330 mg). LCMS (ESI) m/z: [M+H] calcd for C₃₄H₃₇N₃O₄: 552.29; found 552.3.

### Intermediate A-25 and A-26. Synthesis of benzyl (S)-3-methyl-2-((S)-1-oxo-2,7-diazaspiro[4.4]nonan-2-yl)butanoate and benzyl (S)-3-methyl-2-((R)-1-oxo-2,7-diazaspiro[4.4]nonan-2-yl)butanoate

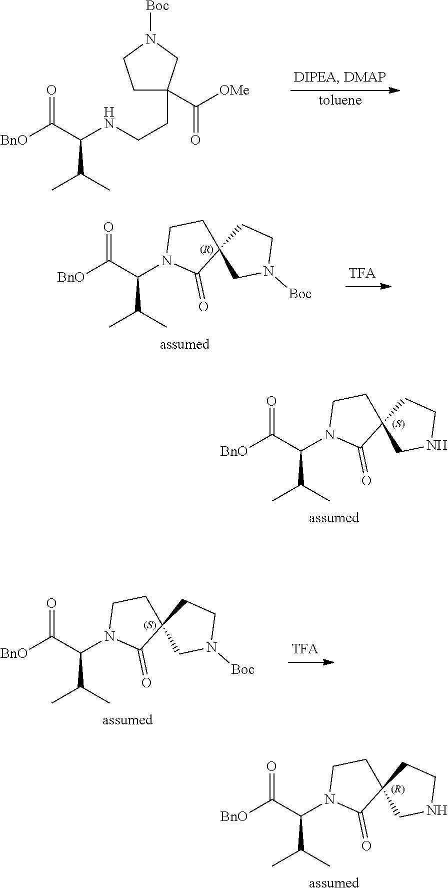

### Step 1: Synthesis of tert-butyl (R)-7-((S)-1-(benzyloxy)-3-methyl-1-oxobutan-2-yl)-6-oxo-2,7-diazaspiro[4.4]nonane-2-carboxylate and tert-butyl (S)-7-((S)-1-(benzyloxy)-3-methyl-1-oxobutan-2-yl)-6-oxo-2,7-diazaspiro[4.4]nonane-2-carboxylate

To a mixture of 1-(tert-butyl) 3-methyl 3-(2-(((S)-1-(benzyloxy)-3-methyl-1-oxobutan-2-yl)amino)ethyl)pyrrolidine-1,3-dicarboxylate (4.50 g, 9.728 mmol) and DIPEA (16.6 mL, 97.28 mmol) in toluene (50 mL) was added DMAP (1.19 g, 9.728 mmol) and then mixture was heated to 80° C. After 24 h the reaction was cooled to room temperature and concentrated under reduced pressure. Purification by reverse phase chromatography (10→50% MeCN/H₂O, 0.1% HCO₂H). The diastereomers were then separated by chiral prep-SFC (30% EtOH/CO₂) to afford tert-butyl (R)-7-((S)-1-(benzyloxy)-3-methyl-1-oxobutan-2-yl)-6-oxo-2,7-diazaspiro[4.4]nonane-2-carboxylate (1.0 g, 32% yield, LCMS (ESI) m/z: [M+H] calcd for C₂₄H₃₄N₂O₅: 431.26; found 431.2) and tert-butyl (S)-7-((S)-1-(benzyloxy)-3-methyl-1-oxobutan-2-yl)-6-oxo-2,7-diazaspiro[4.4]nonane-2-carboxylate carboxylate (1.0 g, 32% yield, LCMS (ESI) m/z: [M+H] calcd for C₂₄H₃₄N₂O₅: 431.26; found 431.2).

### Step 2: Synthesis of benzyl (S)-3-methyl-2-((S)-1-oxo-2,7-diazaspiro[4.4]nonan-2-yl)butanoate

To a solution of tert-butyl (5R)-7-[(2S)-1-(benzyloxy)-3-methyl-1-oxobutan-2-yl]-6-oxo-2,7-diazaspiro[4.4] nonane-2-carboxylate (1.40 g, 3.25 mmol) in DCM (14 mL) at 0° C. was added TFA (5.0 mL, 67.3 mmol). The resulting mixture was stirred at 0° C. for 1 h and was then concentrated under reduced pressure. The mixture was diluted with H₂O (20 mL) and was basified to pH 8 with sat. NaHCO₃ (aq.) at 0° C. The resulting mixture was extracted with EtOAc (3×50 mL) and combined organic layers were washed with brine (40 mL), dried over Na₂SO₄, filtered, and concentrated under reduced pressure to afford the desired product (1.4 g, crude). LCMS (ESI) m/z: [M+H] calcd for C₁₉H₂₆N₂O₃: 331.20; found 331.2).

### Step 3: Synthesis of benzyl (S)-3-methyl-2-((R)-1-oxo-2,7-diazaspiro[4.4]nonan-2-yl)butanoate

To a solution of tert-butyl (5S)-7-[(2S)-1-(benzyloxy)-3-methyl-1-oxobutan-2-yl]-6-oxo-2,7-diazaspiro[4.4] nonane-2-carboxylate (1.0 g, 2.3 mmol) in DCM (10 mL) at 0° C. was added TFA (4.0 mL, 53.9 mmol). The resulting mixture was stirred at 0° C. for 1 h and was then concentrated under reduced pressure. The mixture was diluted with H₂O (10 mL) and was basified to pH 8 with sat. NaHCO₃ (aq.) at 0° C. The resulting mixture was extracted with EtOAc (3×20 mL) and combined organic layers were washed with brine (20 mL), dried over Na₂SO₄, filtered, and concentrated under reduced pressure to afford the desired product (1.0 g, crude). LCMS (ESI) m/z: [M+H] calcd for C₁₉H₂₆N₂O₃: 331.20; found 331.1).

### Intermediate A-27. Synthesis of (2S)-3-methyl-2-(1-oxo-7-((R)-1-tritylaziridine-2-carbonyl)-2,7-diazaspiro[4.4]nonan-2-yl)butanoic acid

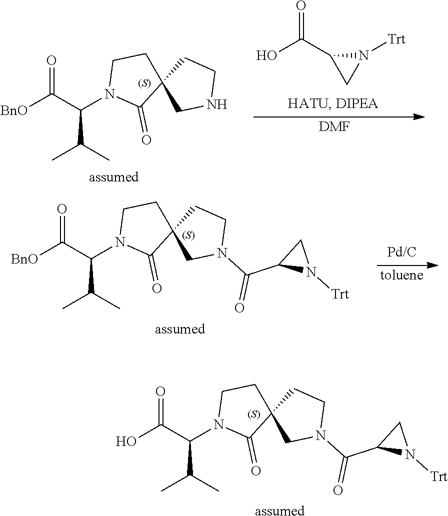

### Step 1: Synthesis of benzyl (S)-3-methyl-2-((S)-1-oxo-7-((R)-1-tritylaziridine-2-carbonyl)-2,7-diazaspiro[4.4]nonan-2-yl)butanoate

To a solution of benzyl (S)-3-methyl-2-((S)-1-oxo-2,7-diazaspiro[4.4]nonan-2-yl)butanoate (400 mg, 1.2 mmol) and DIPEA (1.1 mL, 6.1 mmol) in DMF (5.0 mL) at 0° C. was added (R)-1-tritylaziridine-2-carboxylic acid (480 mg, 1.5 mmol) and HATU (550 mg, 1.5 mmol). The resulting mixture was stirred for 1 h then purified by reverse phase chromatography (15→80% MeCN/H₂O, 0.5% NH₄HCO₃) to afford the desired product (500 mg, 57% yield). LCMS (ESI) m/z: [M+H] calcd for C₄₁H₄₃N₃O₄: 642.34; found 642.3.

### Step 2: Synthesis of (2S)-3-methyl-2-(1-oxo-7-((R)-1-tritylaziridine-2-carbonyl)-2,7-diazaspiro[4.4]nonan-2-yl)butanoic acid

A solution of benzyl (S)-3-methyl-2-((S)-1-oxo-7-((R)-1-tritylaziridine-2-carbonyl)-2,7-diazaspiro[4.4]nonan-2-yl)butanoate (450 mg, 0.70 mmol) and Pd/C (120 mg, 1.13 mmol) in toluene (30 mL) at 50° C. was stirred under a hydrogen atmosphere (1 atm). The mixture was stirred for 3 h and then was filtered, and the filter cake was washed with MeOH (3×30 mL). The filtrate was concentrated under reduced pressure to afford the desired product (430 mg, crude). LCMS (ESI) m/z: [M+H] calcd for C₃₄H₃₇N₃O₄: 552.29; found 552.3.

### Intermediate 28. Synthesis of (S)-3-methyl-2-((R)-1-oxo-7-((R)-1-tritylaziridine-2-carbonyl)-2,7-diazaspiro[4.4]nonan-2-yl)butanoic acid

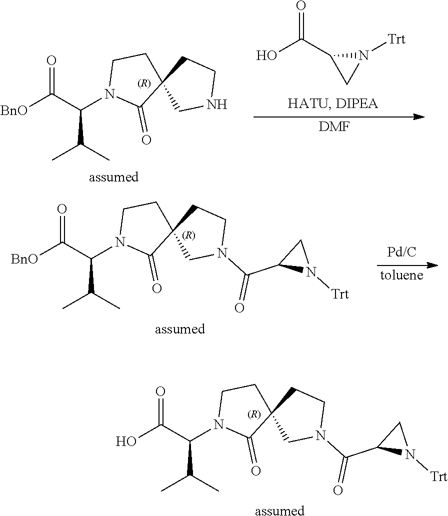

### Step 1: Synthesis of benzyl (S)-3-methyl-2-((R)-1-oxo-7-((R)-1-tritylaziridine-2-carbonyl)-2,7-diazaspiro[4.4]nonan-2-yl)butanoate

To a solution of benzyl (S)-3-methyl-2-((R)-1-oxo-2,7-diazaspiro[4.4]nonan-2-yl)butanoate (500 mg, 1.5 mmol) and DIPEA (1.3 mL, 7.6 mmol) in DMF (7.0 mL) at 0° C. was added (R)-1-tritylaziridine-2-carboxylic acid (550 mg, 1.7 mmol) and HATU (630 mg, 1.7 mmol). The resulting mixture was stirred for 1 h then purification by reverse phase chromatography (10→80% MeCN/H₂O, 0.5% NH₄HCO₃) afforded desired product (700 mg, 64% yield) as an off-white solid. LCMS (ESI) m/z: [M+H] calcd for C₄₁H₄₃N₃O₄: 642.34; found 642.3.

### Step 2: Synthesis of (S)-3-methyl-2-((R)-1-oxo-7-((R)-1-tritylaziridine-2-carbonyl)-2,7-diazaspiro[4.4]nonan-2-yl)butanoic acid

A solution of benzyl (S)-3-methyl-2-((R)-1-oxo-7-((R)-1-tritylaziridine-2-carbonyl)-2,7-diazaspiro[4.4]nonan-2-yl)butanoate (650 mg, 0.70 mmol) and Pd/C (140 mg, 1.3 mmol) in toluene (30 mL) at 50° C. was stirred under a hydrogen atmosphere (1 atm). The mixture was stirred for 3 h and then was filtered, and the filter cake was washed with MeOH (3×30 mL). The filtrate was concentrated under reduced pressure to afford the desired product (550 mg, crude). LCMS (ESI) m/z: [M+H] calcd for C₃₄H₃₇N₃O₄: 552.29; found 552.3.

### Intermediate A-29. Synthesis of (S)-2-((R)-7-(ter-butoxycarbonyl)-1-oxo-2,7-diazaspiro[4.4]nonan-2-yl)-3-methylbutanoic acid

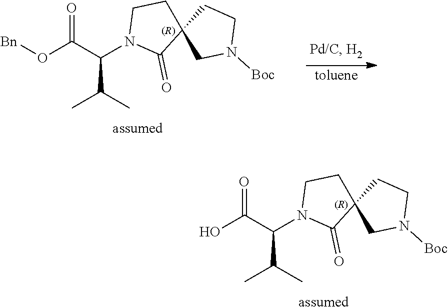

To a solution of tert-butyl (R)-7-((S)-1-(benzyloxy)-3-methyl-1-oxobutan-2-yl)-6-oxo-2,7-diazaspiro[4.4]nonane-2-carboxylate (600 mg, 1.4 mmol) in toluene (20 mL) was added Pd/C (120 mg, 1.1 mmol). The reaction mixture was heated at 50° C. and stirred under a hydrogen atmosphere (1 atm) for 3 h. The mixture was filtered, and the filter cake was washed with MeOH (3×20 mL). The filtrate was concentrated under reduced pressure to afford the desired product (550 mg, crude). LCMS (ESI) m/z: [M−H] calcd for C₁₇H₂₈N₂O₅: 339.19; found 339.1.

### Intermediate A-30. Synthesis of (S)-2-((S)-7-(tert-butoxycarbonyl)-1-oxo-2,7-diazaspiro[4.4]nonan-2-yl)-3-methylbutanoic acid

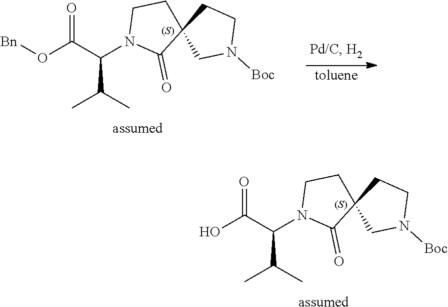

To a solution of tert-butyl (S)-7-((S)-1-(benzyloxy)-3-methyl-1-oxobutan-2-yl)-6-oxo-2,7-diazaspiro[4.4]nonane-2-carboxylate (550 mg, 1.3 mmol) in toluene (30 mL) was added Pd/C (120 mg, 1.1 mmol). The reaction mixture was heated at 50° C. and stirred under a hydrogen atmosphere (1 atm) for 3 h. The mixture was filtered, and the filter cake was washed with MeOH (3×20 mL). The filtrate was concentrated under reduced pressure to afford the desired product (550 mg, crude). LCMS (ESI) m/z: [M−H] calcd for C₁₇H₂₈N₂O₅: 339.19; found 339.2.

### Intermediate A-31. Synthesis of (R)-3-methyl-2-(((S)—N-methyl-1-tritylaziridine-2-carboxamido)methyl)butanoic acid

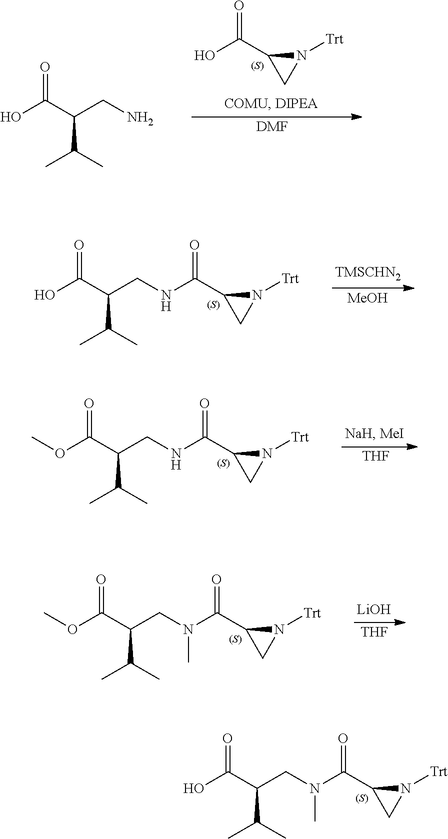

### Step 1: Synthesis of (R)-3-methyl-2-(((S)-1-tritylaziridine-2-carboxamido)methyl)butanoic acid

To a solution of (S)-1-tritylaziridine-2-carboxylic acid (1 g, 2.9 mmol) in DMF (10 mL) at 0° C. was added DIPEA (2.5 mL, 14.55 mmol) followed by COMU (1.12 g, 2.62 mmol). The resulting mixture was stirred for 20 min and (R)-2-(aminomethyl)-3-methylbutanoic acid (382.0 mg, 2.91 mmol) was added. The resulting mixture was warmed to room temperature and stirred for an additional 2 h. The reaction mixture was then quenched with H₂O and the aqueous layer was extracted with EtOAc. The combined organic layers were washed with brine, dried over Na₂SO₄, filtered, and concentrated under reduced pressure. The crude residue was purified by reverse phase chromatography (30→70% MeCN/H₂O+0.1% NH₄HCO₃) to afford the desired product (850 mg, 63% yield). LCMS (ESI) m/z: [M−H] calcd for C₂₅H₃₀N₂O₃: 441.22; found 441.2.

### Step 2: Synthesis of methyl (R)-3-methyl-2-(((S)-1-tritylaziridine-2-carboxamido)methyl)butanoate

To a solution of (R)-3-methyl-2-(((S)—N-methyl-1-tritylaziridine-2-carboxamido)methyl)butanoic acid (840.0 mg, 1.90 mmol) in MeOH (5.0 mL) at 0° C. was added TMSCHN₂ (10.0 mL, 0.45 mmol). The resulting mixture was warmed to room temperature and stirred for 2 h, at which point the reaction mixture was concentrated under reduced pressure. The residue was purified by reverse phase chromatography (30→80% MeCN/H₂O+0.1% NH₄HCO₃) to afford the desired product (450 mg, 52% yield). LCMS (ESI) m/z: [M−H] calcd for C₂₉H₃₂N₂O₃: 455.23; found 455.1.

### Step 3: Synthesis of methyl (R)-3-methyl-2-(((S)—N-methyl-1-tritylaziridine-2-carboxamido)methyl)butanoate

To a solution of methyl (R)-3-methyl-2-(((S)-1-tritylaziridine-2-carboxamido)methyl)butanoate (440.0 mg, 0.96 mmol) in THF (5.0 mL) at 0° C. was added NaH (46.25 mg, 1.93 mmol). The resulting mixture was stirred for 30 min and then Mel (1.37 g, 9.65 mmol) was added. The resulting mixture was warmed to room temperature and stirred for an additional 4 h. The reaction mixture was then quenched with H₂O and the aqueous layer was extracted with EtOAc (3×300 mL). The combined organic layers were washed with brine (3×200 mL), dried over Na₂SO₄, filtered, and concentrated under reduced pressure. The residue was purified by reverse phase chromatography (10→90% MeCN/H₂O+0.1% NH₄HCO₃) to afford the desired product (340 mg, 75% yield). LCMS (ESI) m/z: [M+H] calcd for C₃₀H₃₄N₂O₃: 471.26; found 471.3.

### Step 4: Synthesis of (R)-3-methyl-2-(((S)—N-methyl-1-tritylaziridine-2-carboxamido)methyl)butanoic acid

To a solution of methyl (R)-3-methyl-2-(((S)—N-methyl-1-tritylaziridine-2-carboxamido)methyl)butanoate (340.0 mg, 0.72 mmol) in MeOH (3.0 mL) and H₂O (3.0 mL) was added LiOH·H₂O (242.5 mg, 5.78 mmol). The resulting mixture was stirred for 16 h at room temperature and was then acidified to pH 4 with KHSO₄ (1 N). The resulting mixture was extracted with EtOAc (3×300 mL) and the combined organic layers were washed with brine (3×300 mL), dried over Na₂SO₄, filtered, and concentrated under reduced pressure. The residue was purified by reverse phase chromatography (10→80% MeCN/H₂O+0.1% NH₄HCO₃) to afford the desired product (260 mg). LCMS (ESI) m/z: [M+H] calcd for C₂₉H₃₂N₂O₃: 455.23; found 455.1.

### Intermediate A-32. Synthesis of N-methyl-N-(1-((R)-1-tritylaziridine-2-carbonyl)piperidine-4-carbonyl)-L-valine

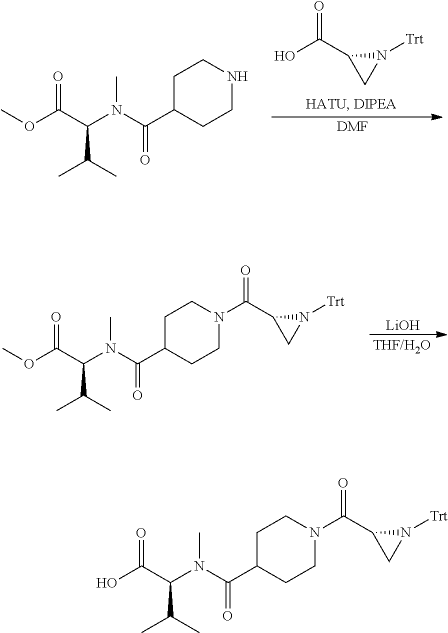

### Step 1: Synthesis of methyl N-methyl-N-(1-((R)-1-tritylaziridine-2-carbonyl)piperidine-4-carbonyl)-L-valinate

To a mixture of methyl N-methyl-N-(piperidine-4-carbonyl)-L-valinate (750 mg, 2.93 mmol) and (R)-1-tritylaziridine-2-carboxylic acid (1.13 g, 3.43 mmol) in DMF (7 mL) at 0° C. was added DIPEA (2.50 mL, 14.62 mmol) followed by HATU (2.20 g, 5.79 mmol) in portions. The resulting mixture was warmed to room temperature and stirred for 3 h. The reaction mixture was diluted with EtOAc (300 mL) and the mixture was washed with brine (2×150 mL), dried with Na₂SO₄, filtered, and concentrated under reduced pressure. Purification by normal phase chromatography (50% EtOAc/hexanes) afforded the desired product (1.5 g, 90.3% yield). LCMS (ESI) m/z: [M+H] calcd for C₃₅H₄₁N₃O₄: 568.32; found 568.3.

### Step 2: Synthesis of N-methyl-N-(1-((R)-1-tritylaziridine-2-carbonyl)piperidine-4-carbonyl)-L-valine

To a solution of methyl N-methyl-N-(1-((R)-1-tritylaziridine-2-carbonyl)piperidine-4-carbonyl)-L-valinate (500 mg, 0.881 mmol) in THF (5 mL) at 0° C. was added a solution of LiOH (111 mg, 2.64 mmol) in H₂O (2.6 mL). The resulting mixture was warmed to room temperature and stirred for 4 h. The reaction mixture was diluted with H₂O (300 mL) and acidified to pH 5 with 1M HCl. The resulting mixture was extracted with DCM (3×100 mL) and the combined organic layers were washed with brine (2×150 mL), dried with Na₂SO₄, filtered, and concentrated under reduced pressure to afford the desired product (600 mg, crude) which was used without further purification. LCMS (ESI) m/z: [M−H] calcd for C₃₄H₃₉N₃O₄: 552.29; found 552.3.

### Intermediate A-33. Synthesis of N-methyl-N-(1-((S)-1-tritylaziridine-2-carbonyl)piperidine-4-carbonyl)-L-valine

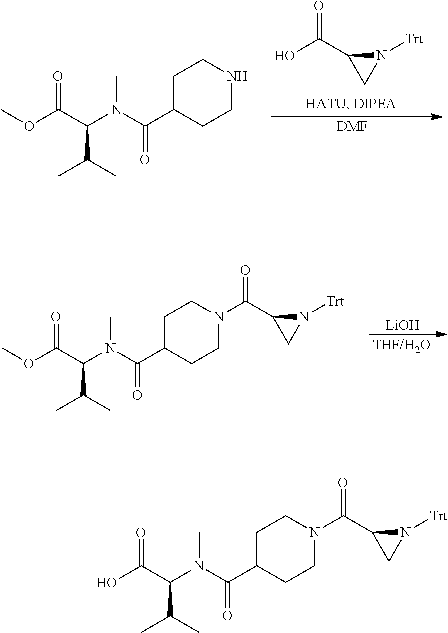

### Step 1: Synthesis of methyl N-methyl-N-(1-((S)-1-tritylaziridine-2-carbonyl)piperidine-4-carbonyl)-L-valinate

To a mixture of methyl N-methyl-N-(piperidine-4-carbonyl)-L-valinate (0.90 g, 3.511 mmol) and (S)-1-tritylaziridine-2-carboxylic acid (2.31 g, 7.022 mmol) in DMF (10 mL) at 0° C. was added DIPEA (3.06 mL, 17.57 mmol) and HATU (2.67 g, 7.022 mmol). The resulting mixture was warmed to room temperature and stirred for 2 h. The reaction mixture was diluted with EtOAc (50 mL) and the mixture was washed with H₂O, brine (100 mL), dried with Na₂SO₄, filtered, and concentrated under reduced pressure. Purification by normal phase chromatography (100% EtOAc) afforded the desired product (1.47 g, 73.7% yield). LCMS (ESI) m/z: [M+H] calcd for C₃₅H₄₁N₃O₄: 568.32; found 568.3.

### Step 2: Synthesis of N-methyl-N-(1-((S)-1-tritylaziridine-2-carbonyl)piperidine-4-carbonyl)-L-valine

To a solution of methyl N-methyl-N-(1-((S)-1-tritylaziridine-2-carbonyl)piperidine-4-carbonyl)-L-valinate (1.0 g, 1.76 mmol) in THF (15 mL) at 0° C. was added a solution of LiOH (370 mg, 8.80 mmol) in H₂O (15 mL). The resulting mixture was warmed to room temperature and stirred for 3 h. The reaction mixture was acidified to pH 6 with 1M HCl. The aqueous layer was extracted with EtOAc (2×50 mL) and the combined organic layers were dried with Na₂SO₄, filtered, and concentrated under reduced pressure to afford the desired product (1.33 g, crude) which was used without further purification. LCMS (ESI) m/z: [M+H] calcd for C₃₄H₃₉N₃O₄: 554.30; found 554.3.

### Intermediate A-34. Synthesis of sodium (R)-1-methyl-5-(1-tritylaziridine-2-carboxamido)-1H-imidazole-2-carboxylate

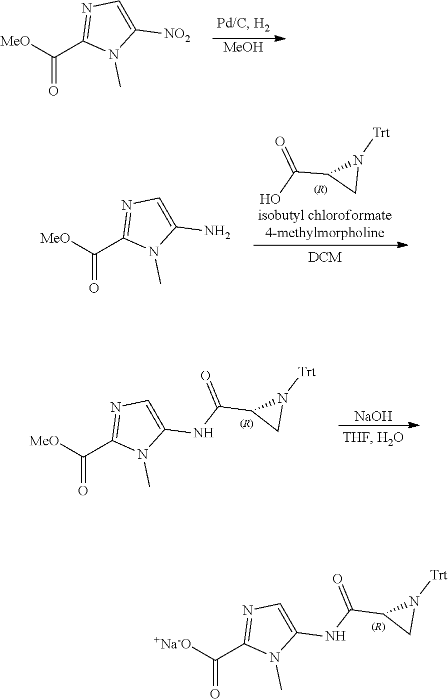

### Step 1: Synthesis of methyl 5-amino-1-methyl-1H-imidazole-2-carboxylate

To a mixture of methyl 1-methyl-5-nitro-1H-imidazole-2-carboxylate (1.0 g, 5.401 mmol) in MeOH (15 mL) was added Pd/C (500 mg). The resulting mixture was placed under an atmosphere of H₂ (1 atm) and stirred for 3 h. The mixture was filtered, the filter cake was washed with MeOH (3×20 mL), and the filtrate was concentrated under reduced pressure to afford the desired product (1.0 g, crude). LCMS (ESI) m/z: [M+H] calcd for C₆H₉N₃O₂: 156.08; found 156.1.

### Step 2: Synthesis of methyl (R)-1-methyl-5-(1-tritylaziridine-2-carboxamido)-1H-imidazole-2-carboxylate

To a mixture of (R)-1-tritylaziridine-2-carboxylic acid (2.55 g, 7.741 mmol) in DCM (12.0 mL) at 0° C. was added a solution of isobutyl chloroformate (845.06 mg, 6.187 mmol) and N-methylmorpholine (1.04 g, 10.282 mmol) in DCM in portions over 30 min. To the resulting mixture was added methyl 5-amino-1-methyl-1H-imidazole-2-carboxylate (800.0 mg, 5.156 mmol). The mixture was stirred at room temperature overnight then diluted with DCM (300 mL) and washed with H₂O (3×100 mL). The organic layer was washed with brine (2×150 mL), dried with Na₂SO₄, filtered, and concentrated under reduced pressure. Purification by silica gel column chromatography (25% EtOAc/hexanes) afforded the desired product (1.2 g, 49.9% yield). LCMS (ESI) m/z: [M+H] calcd for C₂₈H₂₆N₄O₃: 467.21; found 467.2.

### Step 3: Synthesis of sodium (R)-1-methyl-5-(1-tritylaziridine-2-carboxamido)-1H-imidazole-2-carboxylate

To a mixture of methyl (R)-1-methyl-5-(1-tritylaziridine-2-carboxamido)-1H-imidazole-2-carboxylate (300 mg, 0.643 mmol) in THF (3 mL) was added a solution of NaOH (38.58 mg, 0.965 mmol) in H₂O. The resulting mixture was stirred for 2 h and then concentrated under reduced pressure to afford the desired product (400 mg, crude). LCMS (ESI) m/z: [M+H] calcd for C₂₇H₂₄N₄O₃: 453.19; found 453.2.

### Intermediate A-35. Synthesis of (S)-1-methyl-5-(1-tritylaziridine-2-carboxamido)-1H-imidazole-2-carboxylic acid

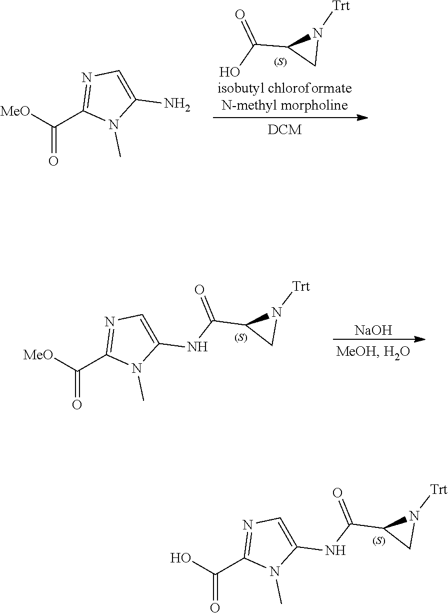

### Step 1: Synthesis of methyl (S)-1-methyl-5-(1-tritylaziridine-2-carboxamido)-1H-imidazole-2-carboxylate

To a mixture of (S)-1-tritylaziridine-2-carboxylic acid (1.18 g, 3.577 mmol) in DCM (15 mL) at 0° C. was added isobutyl chloroformate (423.41 mg, 3.100 mmol) and N-methylmorpholine (0.39 mL, 3.862 mmol) dropwise. The resulting mixture was stirred at 0° C. for 1 h then methyl 5-amino-1-methyl-1H-imidazole-2-carboxylate (370.0 mg, 2.385 mmol) was added. The mixture was warmed to room temperature and stirred overnight. The reaction was quenched with sat. NaHCO₃ at 0° C. and the resulting mixture was extracted with DCM (2×100 mL). The combined organic layers were washed with brine (150 mL), dried with Na₂SO₄, filtered, and concentrated under reduced pressure. Purification by silica gel column chromatography (100% EtOAc) afforded the desired product (380 mg, 34.2% yield). LCMS (ESI) m/z: [M+H] calcd for C28H26N4O3: 467.21; found 467.3.

### Step 2: Synthesis of (S)-1-methyl-5-(1-tritylaziridine-2-carboxamido)-1H-imidazole-2-carboxylic acid

To a mixture of methyl (S)-1-methyl-5-(1-tritylaziridine-2-carboxamido)-1H-imidazole-2-carboxylate (380.0 mg, 0.815 mmol) in MeOH (5 mL) at 0° C. was added NaOH (146.60 mg, 3.665 mmol) in H₂O (3.6 mL) dropwise. The resulting mixture was warmed to room temperature and stirred for 6 h then was acidified to pH 6 with 1M HCl. The resulting mixture was extracted with DCM (2×100 mL), and the combined organic layers were washed with brine (150 mL), dried with Na₂SO₄, filtered, and concentrated under reduced pressure to afford the desired product (350 mg, crude). LCMS (ESI) m/z: [M−H] calcd for C₂₇H₂₄N₄O₃: 451.17; found 451.1.

### Intermediate A-36 and A-37. Synthesis of (R)—N-methyl-N-((1-methylaziridin-2-yl)sulfonyl)glycine and (S)—N-methyl-N-((1-methylaziridin-2-yl)sulfonyl)glycine

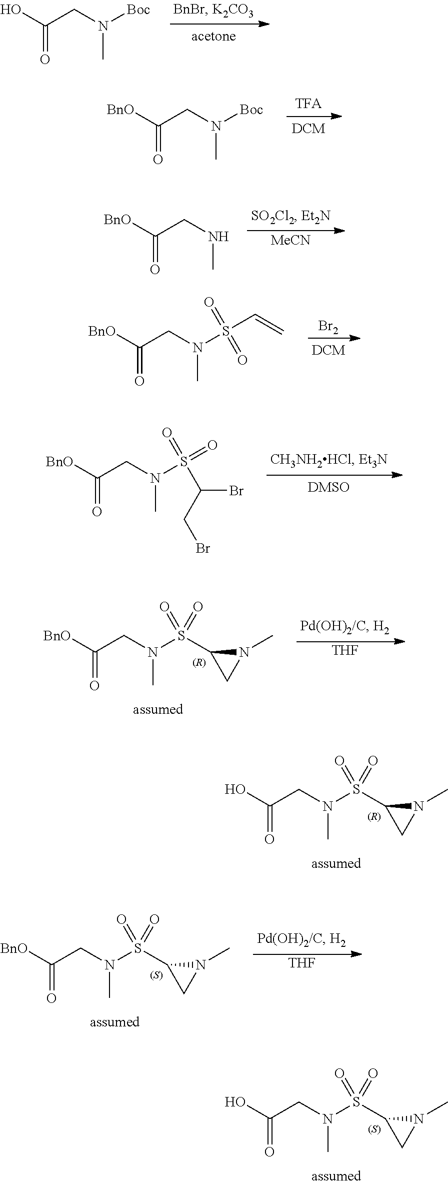

### Step 1: Synthesis of benzyl N-(tert-butoxycarbonyl)-N-methylglycinate

To a stirred mixture of [(tert-butoxycarbonyl)(methyl)amino]acetic acid (15. g, 79.28 mmol) in acetone (150 mL) was added BnBr (14.14 mL, 82.70 mmol) and K₂CO₃ (21.91 g, 158.55 mmol) in portions at 0° C. The resulting mixture was stirred for 4 h at room temperature. The resulting mixture was filtered, the filter cake was washed with acetone (3×100 mL), and the filtrate was concentrated under reduced pressure. The residue was purified by silica gel column chromatography (33% EtOAc/pet. ether) to afford the desired product (15.2 g, 68.6% yield). LCMS (ESI) m/z: [M+Na] calcd for C₁₅H₂₁NO₄: 302.14; found 302.0.

### Step 2: Synthesis of benzyl methylglycinate

To a stirred solution of benzyl N-(tert-butoxycarbonyl)-N-methylglycinate (10.0 g, 35.80 mmol) in DCM (100 mL) was added TFA (50 mL) dropwise at 0° C. The resulting mixture was stirred for 1 h at 0° C. and then the resulting mixture was concentrated under reduced pressure to afford the desired product (7.80 g, crude). LCMS (ESI) m/z: [M+H] calcd for C₁₀H₁₃NO₂: 180.10; found 179.1.

### Step 3: Synthesis of benzyl N-methyl-N-(vinylsulfonyl)glycinate

To a solution of benzyl methylglycinate (15.60 g, 87.04 mmol) and Et₃N (36.4 mL, 261.1 mmol) in MeCN (300 mL) at −70° C. was added a solution of 2-chloroethanesulfonyl chloride (17.03 g, 104.47 mmol) in MeCN (150 mL). The resulting mixture was warmed to room temperature and stirred for 20 min. The reaction mixture was cooled −50° C. and additional Et₃N (36.4 mL, 261.1 mmol) was added to reaction mixture. The reaction mixture was warmed to room temperature and stirred for 1 h. The reaction was then quenched with H₂O at 0° C. The mixture was acidified to pH 6 with 1 M HCl aq and was then extracted with DCM (800 mL), dried over Na₂SO₄, filtered, and concentrated under reduced pressure. The residue was purified by silica gel column chromatography (33% EtOAc/pet. ether) to afford the desired product (7.53 g, 32.1% yield). LCMS (ESI) m/z: [M+H₂O] calcd for C₁₂H₁₅NO₄S: 287.08; found 287.2.

### Step 4: Synthesis of benzyl N-((1,2-dibromoethyl)sulfonyl)-N-methylglycinate

To a solution of benzyl N-methyl-N-(vinylsulfonyl)glycinate (5.58 g, 20.7 mmol) in DCM (50 mL) at −20° C. was added a solution of Br₂ (1.06 mL, 6.64 mmol) in DCM (10 mL). The resulting mixture was warmed to room temperature and stirred overnight. The reaction mixture was then cooled to 0° C. and quenched with sat. aq. Na₂S₂O₃ (30 mL). The resulting mixture was washed with sat. aq. Na₂HCO₃ and then extracted with DCM (2×200 mL), the combined organic layers dried over Na₂SO₄, filtered, and concentrated under reduced pressure. The residue was purified by silica gel column chromatography (33% EtOAc/pet. ether) to afford the desired product (5.1 g, 57.4% yield). LCMS (ESI) m/z: [M+H₂O] calcd for C₁₂H₁₅Br₂NO₄S: 444.92; found 444.9.

### Step 5: Synthesis of benzyl (R)—N-methyl-N-((1-methylaziridin-2-yl)sulfonyl)glycinate and benzyl (S)—N-methyl-N-((1-methylaziridin-2-yl)sulfonyl)glycinate

To a stirred solution of benzyl N-((1,2-dibromoethyl)sulfonyl)-N-methylglycinate (7.20 g, 16.78 mmol) and methylamine hydrochloride (3.39 g, 50.2 mmol) in DMSO (750 mL) was added Et₃N (23.32 mL, 230.47 mmol). The resulting mixture was stirred for 2 h at room temperature then heated to 75° C. and stirred overnight. The reaction mixture was cooled to room temperature and extracted with EtOAc (2 30×1000 mL). The combined organic layers were washed with H₂O (1500 mL) and brine (1500 mL), dried over Na₂SO₄, filtered, and concentrated under reduced pressure. The residue was purified by silica gel column chromatography (EtOAc) to afford a mixture of diastereomers. The diastereomers were separated by prep-SFC (10% EtOH/Hex) to afford benzyl (R)—N-methyl-N-((1-methylaziridin-2-yl)sulfonyl)glycinate (500 mg, 31.3% yield) and benzyl (S)—N-methyl-N-((1-methylaziridin-2-yl)sulfonyl)glycinate (600 mg, 37.5% yield). LCMS (ESI) m/z: [M+H] calcd for C₁₃H₁₈BN₂O₄S: 299.11; found 299.0.

### Step 6: Synthesis of (R)—N-methyl-N-((1-methylaziridin-2-yl)sulfonyl)glycine

A suspension of benzyl (R)—N-methyl-N-((1-methylaziridin-2-yl)sulfonyl)glycinate (300.0 mg) and Pd(OH)₂/C (150.0 mg) in THF at room temperature was stirred under an atmosphere of hydrogen (1 atm) for 3 h. The mixture was filtered, the filter cake was washed with MeOH (3×20 mL), and the filtrate was concentrated under reduced pressure to afford the desired product (206 mg, crude). LCMS (ESI) m/z: [M+H] calcd for C₆H₁₂N₂O₄S: 209.06; found 209.0.

### Step 7: Synthesis of (S)—N-methyl-N-((1-methylaziridin-2-yl)sulfonyl)glycine

A suspension of benzyl (R)—N-methyl-N-((1-methylaziridin-2-yl)sulfonyl)glycinate (300.0 mg, 1.01 mmol) and Pd(OH)₂/C (150.0 mg) in THF at room temperature was stirred under an atmosphere of hydrogen (1 atm) for 3 h. The mixture was filtered, the filter cake was washed with MeOH (3×20 mL), and the filtrate was concentrated under reduced pressure to afford the desired product (216 mg, crude). LCMS (ESI) m/z: [M+H] calcd for C₆H₁₂N₂O₄S: 209.06; found 209.1.

### Intermediate A-38. Synthesis of (2S,3S)-1-(tert-butylsulfinyl)-3-cyclopropylaziridine-2-carboxylic acid

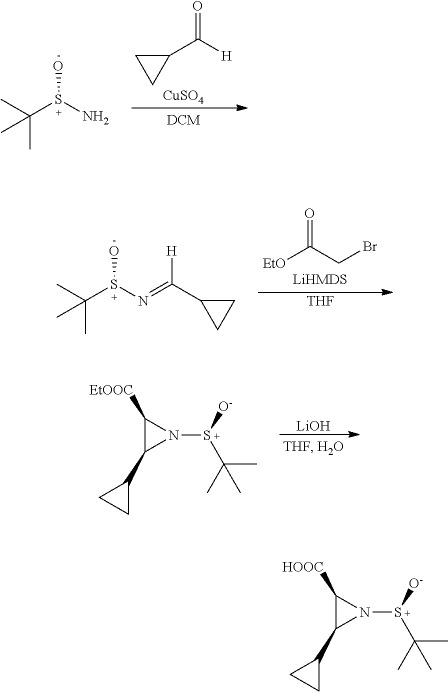

Step 7: Synthesis of (E)-N-(cyclopropylmethylene)-2-methylpropane-2-sulfinamide

To a suspension of (S)-2-methylpropane-2-sulfinamide (4.0 g, 33.0 mmol) and CuSO₄ (15.80 g, 99.01 mmol) in DCM (200.0 mL) was added cyclopropanecarbaldehyde (4.63 g, 66.0 mmol). The resulting mixture was stirred overnight and was then filtered, the filter cake was washed with DCM (3×100 mL), and the filtrate was concentrated under reduced pressure to afford the desired product (3.5 g, 61.2% yield). LCMS (ESI) m/z: [M+H] calcd for C₈H₁₅NOS: 174.10; found 174.1.

### Step 2: Synthesis of ethyl (2S,3S)-1-(tert-butylsulfinyl)-3-cyclopropylaziridine-2-carboxylate

To a solution of ethyl bromoacetate (481.91 mg, 2.886 mmol) in THF (5.0 mL) at −78° C. was added LiHMDS (2.90 mL, 2.90 mmol). The resulting mixture was stirred for 2 h at −78° C. and then a solution of (E)-N-(cyclopropylmethylene)-2-methylpropane-2-sulfinamide (250.0 mg, 1.443 mmol) was added. The resulting mixture was stirred for 2 h at −78° C. and was then was then quenched with H₂O at 0° C. The aqueous layer was extracted with EtOAc (3×50 mL), and the combined organic layers were dried over Na₂SO₄, filtered, and concentrated under reduced pressure. The residue was purified by prep-TLC (17% EtOAc/pet. ether) to afford the desired product (250 mg, 66.8% yield). LCMS (ESI) m/z: [M+H] calcd for C₁₂H₂₁NO₃S: 260.13; found 260.1.

### Step 3: Synthesis of (2S,3S)-1-(tert-butylsulfinyl)-3-cyclopropylaziridine-2-carboxylic acid

A solution of ethyl (2S,3S)-1-(tert-butylsulfinyl)-3-cyclopropylaziridine-2-carboxylate (500.0 mg, 1.928 mmol) in THF (2.0 mL) and H₂O (2.0 mL) at 0° C. was added LiOH·H₂O (121.34 mg, 2.89 mmol). The reaction mixture was stirred for 1 h and was then acidified to pH 6 with 1 M HCl (aq.). The resulting mixture was extracted with EtOAc (2×10 mL) and the combined organic layers were washed with brine (10 mL), dried over Na₂SO₄, filtered, and the filtrate was concentrated under reduced pressure to afford the desired product (400 mg, 89.7% yield). LCMS (ESI) m/z: [M+H] calcd for C₁₀H₁₇NO₃S: 232.10; found 232.0.

### Intermediate A-39. Synthesis of (2R,3R)-3-(methoxymethyl)-1-tritylaziridine-2-carboxylic acid

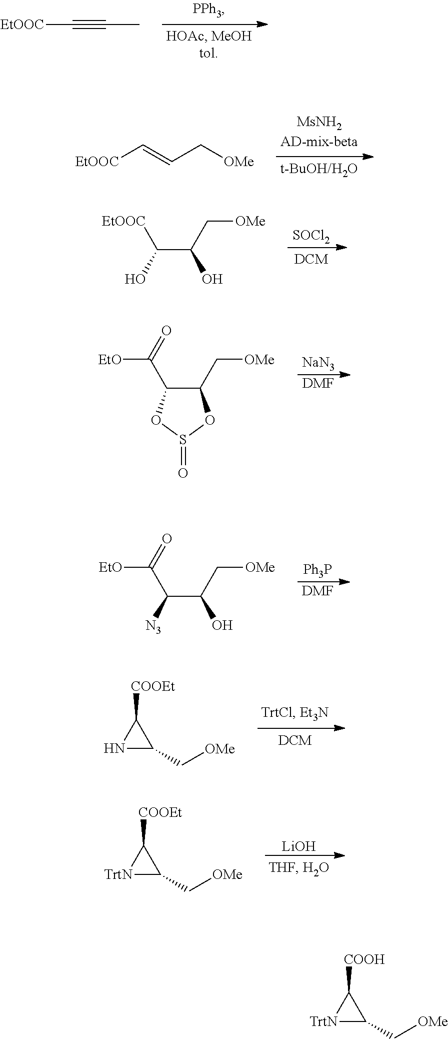

### Step 1: Synthesis of ethyl (E)-4-methoxybut-2-enoate

To a solution of ethyl but-2-ynoate (10.0 g, 89.18 mmol) in MeOH (8.80 mL, 118.594 mmol) and HOAc (1.05 mL, 18.3 mmol) was added a solution of PPh₃ (1.20 g, 4.58 mmol) in toluene (60.0 mL). The resulting solution heated to 110° C. and stirred overnight. The reaction mixture was cooled to room temperature and was then diluted with H₂O (60 mL). The resulting solution was extracted with EtOAc (2×60), and the combined organic layers were washed with brine (2×20 mL), dried over Na₂SO₄, filtered, and concentrated under reduced pressure. The residue was purified by silica gel column chromatography (9% EtOAc/pet. ether) to afford the desired product (4.9 g, 38.1% yield). LCMS (ESI) m/z: [M+H] calcd for C₇H₁₂O₃: 145.09; found 144.9.

### Step 2: Synthesis of ethyl (2S,3R)-2,3-dihydroxy-4-methoxybutanoate

To a solution of ethyl (E)-4-methoxybut-2-enoate (5.0 g, 34.68 mmol), and methanesulfonamide (3.30 g, 34.68 mmol) in t-BuOH (150.0 mL) and H₂O (100.0 mL) was added AD-mix-β (48.63 g, 62.43 mmol). The resulting solution was heated to 30° C. and stirred overnight. The solution was then cooled to room temperature and adjusted to pH 2 with KHSO₄. The resulting solution was extracted with EtOAc (2×100 mL) and the combined organic layers were dried over Na₂SO₄, filtered, and concentrated under reduced pressure to afford the desired product (1.28 g, crude). LCMS (ESI) m/z: [M+H] calcd for C₇H₁₄O₅: 179.09; found 179.0.

### Step 3: Synthesis of ethyl (4S,5R)-5-(methoxymethyl)-1,3,2-dioxathiolane-4-carboxylate 2-oxide

To a solution of ethyl (2S,3R)-2,3-dihydroxy-4-methoxybutanoate (4.10 g, 23.01 mmol) in DCM (20.0 mL) at 0° C. was added SOCl₂ (5.47 g, 45.9 mmol). The resulting mixture was heated to 50° C. and stirred for 3 h. The reaction mixture was then cooled to room temperature and concentrated under reduced pressure to afford the desired product (4.0 g, crude).

### Step 4: Synthesis of ethyl (2R,3S)-2-azido-3-hydroxy-4-methoxybutanoate

To a solution of ethyl (4S,5R)-5-(methoxymethyl)-1,3,2-dioxathiolane-4-carboxylate 2-oxide (4.0 g crude, 17.84 mmol) in DMF (20.0 mL) at 0° C. was added NaN₃ (5.80 g, 89.22 mmol). The resulting mixture was heated to 35° C. and stirred overnight. The reaction mixture was then diluted with H₂O (200 mL) and was extracted with EtOAc (3×50 mL). The combined organic layers were washed with brine (3×50 mL), dried over Na₂SO₄, filtered, and concentrated under reduced pressure. The residue was purified by silica gel column chromatography (17% EtOAc/pet. ether) to afford the desired product (1.0 g, 27.6% yield). LCMS (ESI) m/z: [M+H] calcd for C₇H₁₃N₃O₄: 204.10; found 204.0.

### Step 5: Synthesis of ethyl (2R,3R)-3-(methoxymethyl)aziridine-2-carboxylate

To a solution of ethyl (2R,3S)-2-azido-3-hydroxy-4-methoxybutanoate (1.0 g, 4.92 mmol) in DMF (10 mL) at 0° C. was added PPh₃ (1.29 g, 4.92 mmol) in portions over 30 min. The reaction solution was then warmed to room temperature and stirred for 30 min. The reaction mixture was then heated to 85° C. and stirred until the reaction was complete. The reaction mixture was then concentrated under reduced pressure and purified by prep-TLC (33% EtOAc/pet. ether) to afford the desired product (480 mg, 61.3% yield). LCMS (ESI) m/z: [M+H] calcd for C₇H₁₃NO₃: 160.10; found 160.1.

### Step 6: Synthesis of ethyl (2R,3R)-3-(methoxymethyl)-1-tritylaziridine-2-carboxylate

To a solution of ethyl (2R,3R)-3-(methoxymethyl)aziridine-2-carboxylate (480.0 mg, 3.02 mmol) and Et₃N (2.1 mL, 15.0 mmol) in DCM (10 mL) at 0° C. was added Trt-Cl (1.681 g, 6.031 mmol). The resulting mixture was warmed to room temperature and stirred for 2 h. The mixture was concentrated then concentrated under reduced pressure and the residue was purified by prep-TLC (5% EtOAc/pet. ether) to afford the desired product (700 mg, crude).

### Step 7: Synthesis of (2R,3R)-3-(methoxymethyl)-1-tritylaziridine-2-carboxylic acid

To a solution of ethyl (2R, 3R)-3-(methoxymethyl)-1-(triphenylmethyl)aziridine-2-carboxylate (200.0 mg, 0.498 mmol) in THF (5.0 mL) and H₂O (5 mL) was added LiOH·H₂O (41.81 mg, 0.996 mmol). The resulting solution was stirred at room temperature for 24 h. The mixture was then diluted with H₂O (10 mL) and extracted with EtOAc (20 mL). The aqueous layer was then acidified to pH 7 with sat. aq. NH₄Cl and extracted with EtOAc (2×10 mL). The combined organic layers were dried over Na₂SO₄, filtered, and concentrated under reduced pressure to afford the desired product (60 mg, 32.3% yield). LCMS (ESI) m/z: [M−H] calcd for C₂₄H₂₃NO₃: 372.16; found 372.1.

### Intermediate A-40. Synthesis of (2S,3S)-1-(tert-butylsulfinyl)-3-(4-methoxyphenyl)aziridine-2-carboxylic acid

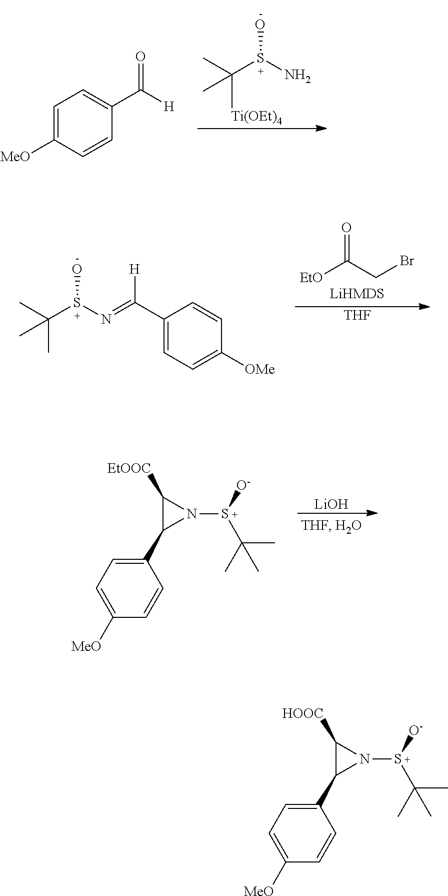

### Step 1: (E)-N-(4-methoxybenzylidene)-2-methylpropane-2-sulfinamide

A solution of (S)-2-methylpropane-2-sulfinamide (2.50 g) and anisaldehyde (2.81 g) in Ti(OEt)₄ (20.0 mL) was stirred at 70° C. for 1 h. The resulting mixture was cooled to room temperature, diluted with EtOAc (60 mL), and then poured into H₂O. The mixture was filtered, and the filter cake was washed with EtOAc (3×50 mL). The resulting mixture was extracted with EtOAc (3×50 mL) and the combined organic layers were washed with brine (50 mL), dried over Na₂SO₄, filtered, and concentrated under reduced pressure. The residue was purified by silica gel column chromatography (25% EtOAc/pet. ether) to afford the desired product (4 g, 81.0% yield). LCMS (ESI) m/z: [M+H] calcd for C₁₂H₁₇NO₂S: 240.11; found 240.1.

### Step 2: Synthesis of ethyl (2S,3S)-1-(tert-butylsulfinyl)-3-(4-methoxyphenyl)aziridine-2-carboxylate

To a solution of ethyl 2-bromoacetate (5.60 g, 33.5 mmol) in THF (100 mL) at −78° C. was added LiHMDS (1M in THF, 34 mL, 33.473 mmol). After 30 min a solution of (E)-N-(4-methoxybenzylidene)-2-methylpropane-2-sulfinamide (4 g, 16.74 mmol) in THF (20 mL) was added. The resulting mixture was stirred at −78° C. for additional 3 h. The reaction was then quenched with sat. aq. NH₄Cl. The mixture was extracted with EtOAc (3×100 mL) and the combined organic layers were washed with brine (50 mL), dried over Na₂SO₄, filtered, and concentrated under reduced pressure. The residue was purified by silica gel chromatography (25% EtOAc/pet. ether) to afford the desired product (2.7 g, 49.6% yield). LCMS (ESI) m/z: [M+H] calcd for C₁₆H₂₃NO₄S: 326.14; found 326.1.

### Step 3: Synthesis of (2S,3S)-1-(tert-butylsulfinyl)-3-(4-methoxyphenyl)aziridine-2-carboxylic acid

To a solution of ethyl (2S,3S)-1-(tert-butylsulfinyl)-3-(4-methoxyphenyl)aziridine-2-carboxylate (80 0.0 mg, 2.68 mmol) in THF (2.0 mL) at 0° C. was added a solution of LiOH·H₂O (309.46 mg, 7.38 mmol) in H₂O (3.0 mL). The resulting mixture was warmed to room temperature and stirred for 4 h. The mixture w as then acidified to pH 6 with sat. aq. NH₄Cl and then extracted with EtOAc (3×50 mL). The combined organic layers were dried over Na₂SO₄, filtered. and concentrated under reduced pressure to afford the desi red product (690 mg, 94.4% yield). LCMS (ESI) m/z: [M−H] calcd for C₁₄H₁₉NO₄S: 296.10; found 296.2.

### Intermediate A-41. Synthesis of (2S,3R)-3-(4-methoxyphenyl)aziridine-2-carboxylic acid

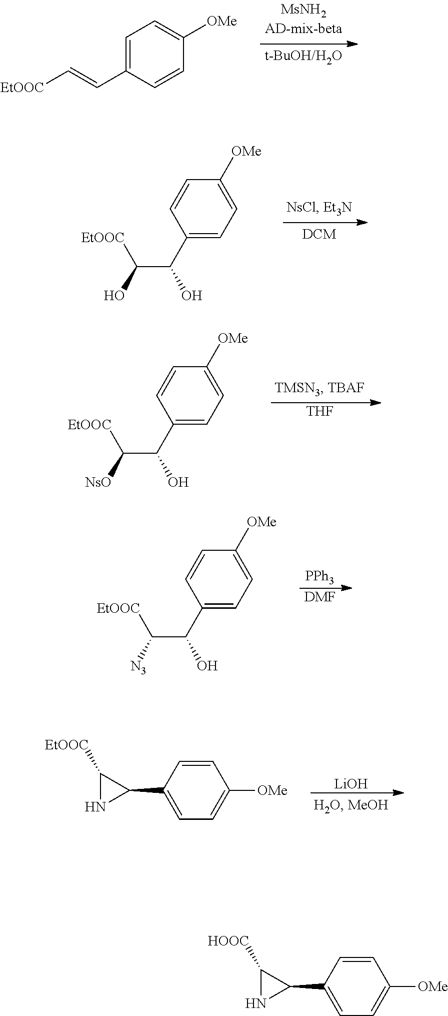

### Step 1: Synthesis of ethyl (2R,3S)-2,3-dihydroxy-3-(4-methoxyphenyl)propanoate

To a solution of ethyl p-methoxycinnamate (5.0 g, 24.24 mmol) in tBuOH (70.0 mL) and H₂O (70.0 mL) at 0° C. was added AD-mix-α (33.80 g, 43.39 mmol) and methanesulfonamide (2.31 mg, 0.024 mmol). The resulting mixture was warmed to room temperature and stirred overnight. The reaction was then cooled to 0° C. and quenched with KHSO₄ (aq.). The mixture was extracted with EtOAc (3×100 mL) and the combined organic layers were washed with brine (2×90 mL), dried over Na₂SO₄, filtered, and concentrated under reduced pressure. The residue was purified by silica gel chromatography (50% EtOAc/pet. ether) to afford the desired product (5.7 g, 88.1% yield).

### Step 2: Synthesis of ethyl (2R,3S)-3-hydroxy-3-(4-methoxyphenyl)-2-(((4-nitrophenyl)sulfonyl)oxy)propanoate

To a solution of ethyl (2R,3S)-2,3-dihydroxy-3-(4-methoxyphenyl)propanoate (3.0 g, 12.49 mmol) and Et₃N (0.174 mL, 1.249 mmol) in DCM (30.0 mL) at 0° C. was added 4-nitrobenzenesulfonyl chloride (2.76 g, 12.49 mmol). The resulting mixture was stirred for 1 h and was then diluted with H₂O. The mixture was extracted with DCM (3×100 mL) and the combined organic layers were washed with brine (2×100 mL), dried over Na₂SO₄, filtered, and concentrated under reduced pressure. The residue was purified by prep-TLC (50% EtOAc/pet. ether) to afford the desired product (3.8 g, 68.0% yield). LCMS (ESI) m/z: [M+Na] calcd for C₁₈H₁₉NO₉S: 448.07; found 448.2.

### Step 3: Synthesis of ethyl (2S,3S)-2-azido-3-hydroxy-3-(4-methoxyphenyl)propanoate

To a solution of ethyl (2R,3S)-3-hydroxy-3-(4-methoxyphenyl)-2-(((4-nitrophenyl)sulfonyl)oxy)propanoate (1.20 g, 2.82 mmol) in THF at 0° C. was added TBAF (1M in THF, 5.64 mL, 5.64 mmol) and TMSN₃ (648.79 mg, 5.64 mmol). The resulting mixture was heated to at 60° C. and stirred for 16 h. The reaction was then cooled to at 0° C. and quenched with sat. aq. NH₄Cl. The mixture was extracted with EtOAc (3×100 mL) and the combined organic layers were washed with H₂O (2×100 mL), dried over Na₂SO₄, filtered, and concentrated under reduced pressure. The residue was purified by prep-TLC (33% EtOAc/pet. ether) to afford the desired product (540 mg, 70.7% yield).

### Step 4: Synthesis of ethyl (2S,3R)-3-(4-methoxyphenyl)aziridine-2-carboxylate

To a solution of ethyl (2S,3S)-2-azido-3-hydroxy-3-(4-methoxyphenyl)propanoate (440.0 mg, 1.659 mmol) in DMF was added PPh₃ (522.06 mg, 1.99 mmol). The resulting mixture was stirred at room temperature for 30 min and was then heated to 80° C. and stirred overnight. The mixture was then extracted with EtOAc (3×100 mL), and the combined organic layers were washed with H₂O (2×100 mL), dried over anhydrous Na₂SO₄, filtered, and concentrated under reduced pressure. The residue was purified by prep-TLC (25% EtOAc/pet. ether) to afford the desired product (200 mg, 51.8% yield). LCMS (ESI) m/z: [M+H] calcd for C₁₂H₁₅NO₃: 222.12; found 222.1.

### Step 5: Synthesis of (2S,3R)-3-(4-methoxyphenyl)aziridine-2-carboxylic acid

To a solution of ethyl (2S,3R)-3-(4-methoxyphenyl)aziridine-2-carboxylate (200.0 mg, 0.904 mmol) in MeOH and H₂O at 0° C. was added LiOH·H₂O (86.6 mg, 3.62 mmol). The resulting mixture was stirred for 1 h and was then neutralized to pH 7 with HCl (aq.). The mixture was extracted with EtOAc (3×100 mL) and the combined organic layers were washed with H₂O (2×100 mL), dried over Na₂SO₄, filtered, and concentrated under reduced pressure to afford the desired product (180 mg, 97.9% yield). LCMS (ESI) m/z: [M−H] calcd for C₁₀H₁₁NO₃: 192.07; found 192.0.

### Intermediate A-42. Synthesis of (2R,3S)-3-(4-methoxyphenyl)aziridine-2-carboxylic acid

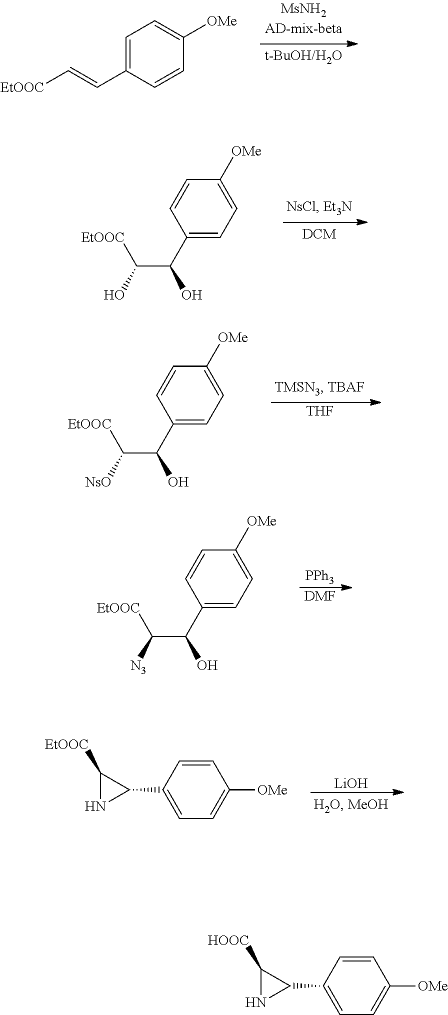

### Step 1: Synthesis of ethyl (2S,3R)-2,3-dihydroxy-3-(4-methoxyphenyl)propanoate

To a solution of ethyl p-methoxycinnamate (5.0 g, 24.24 mmol) in tBuOH (70.0 mL) and H₂O (70.0 mL) at 0° C. was added AD-mix-β (33.80 g, 43.39 mmol) and methanesulfonamide (2.31 mg, 0.024 mmol). The resulting mixture was warmed to room temperature and stirred overnight. The reaction was then cooled to 0° C. and quenched with KHSO₄ (aq.). The mixture was extracted with EtOAc (3×100 mL) and the combined organic layers were washed with brine (2×90 mL), dried over Na₂SO₄, filtered, and concentrated under reduced pressure. The residue was purified by silica gel chromatography (50% EtOAc/pet. ether) to afford the desired product (5.7 g, 88.1% yield).

### Step 2: Synthesis of ethyl (2S,3R)-3-hydroxy-3-(4-methoxyphenyl)-2-(((4-nitrophenyl)sulfonyl)oxy)propanoate

To a solution of ethyl (2S,3R)-2,3-dihydroxy-3-(4-methoxyphenyl)propanoate (5.80 g, 24.14 mmol) and Et₃N (10.1 mL, 72.42 mmol) in DCM (30.0 mL) at 0° C. was added 4-nitrobenzenesulfonyl chloride (5.34 g, 24.1 mmol). The resulting mixture was stirred for 1 h and was then diluted with H₂O. The mixture was extracted with DCM (3×100 mL) and the combined organic layers were washed with brine (2×100 mL), dried over Na₂SO₄, filtered, and concentrated under reduced pressure. The residue was purified by prep-TLC (50% EtOAc/pet. ether) to afford the desired product (7.2 g, 67.0% yield). LCMS (ESI) m/z: [M+H] calcd for C₁₈H₁₉NO₉S: 426.09; found 426.2.

### Step 3: Synthesis of ethyl (2R,3R)-2-azido-3-hydroxy-3-(4-methoxyphenyl)propanoate

To a solution of ethyl (2S,3R)-3-hydroxy-3-(4-methoxyphenyl)-2-(((4-nitrophenyl)sulfonyl)oxy)propanoate (5.0 g, 11.75 mmol) in THF at 0° C. was added TBAF (1M in THF, 23.5 mL, 23.51 mmol) and TMSN₃ (2.7 g, 23.5 mmol). The resulting mixture was heated to at 60° C. and stirred for 16 h. The reaction was then cooled to at 0° C. and quenched with sat. aq. NH₄Cl. The mixture was extracted with EtOAc (3×100 mL) and the combined organic layers were washed with H₂O (2×100 mL), dried over Na₂SO₄, filtered, and concentrated under reduced pressure. The residue was purified by prep-TLC (33% EtOAc/pet. ether) to afford the desired product (2.3 g, 70.1% yield).

### Step 4: Synthesis of ethyl (2R,3S)-3-(4-methoxyphenyl)aziridine-2-carboxylate

To a solution of ethyl (2R,3R)-2-azido-3-hydroxy-3-(4-methoxyphenyl)propanoate (2.30 g, 8.67 mmol) in DMF was added PPh₃ (2.73 g, 10.4 mmol). The resulting mixture was stirred at room temperature for 30 min and was then heated to 80° C. and stirred overnight. The mixture was then extracted with EtOAc (3×100 mL), and the combined organic layers were washed with H₂O (2×100 mL), dried over anhydrous Na₂SO₄, filtered, and concentrated under reduced pressure. The residue was purified by prep-TLC (25% EtOAc/pet. ether) to afford the desired product (1.6 g, 79.2% yield). LCMS (ESI) m/z: [M+H] calcd for C₁₂H₁₅NO₃: 222.12; found 222.1.

### Step 5: Synthesis of (2R,3S)-3-(4-methoxyphenyl)aziridine-2-carboxylic acid

To a solution of ethyl (2S,3R)-3-(4-methoxyphenyl)aziridine-2-carboxylate (200.0 mg, 0.904 mmol) in MeOH and H₂O at 0° C. was added LiOH·H₂O (86.6 mg, 3.62 mmol). The resulting mixture was stirred for 1 h and was then neutralized to pH 7 with HCl (aq.). The mixture was extracted with EtOAc (3×100 mL) and the combined organic layers were washed with H₂O (2×100 mL), dried over Na₂SO₄, filtered, and concentrated under reduced pressure to afford the desired product (180 mg, 97.9% yield). LCMS (ESI) m/z: [M−H] calcd for C₁₀H₁₁NO₃: 192.07; found 192.0.

### Intermediate A-43. Synthesis of (2,3S)-1-((S)-tert-butylsulfinyl)-3-phenylaziridine-2-carboxylic acid

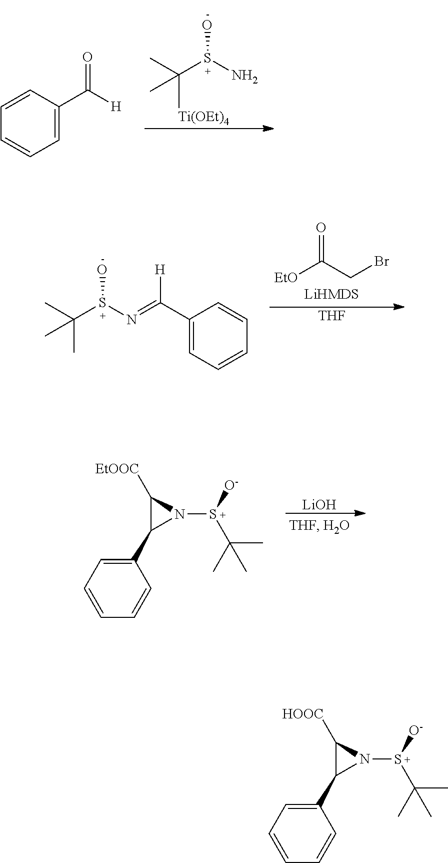

### Step 1: Synthesis of (S,E)-N-benzylidene-2-methylpropane-2-sulfinamide

A solution of (S)-2-methylpropane-2-sulfinamide (2.50 g, 20.6 mmol), titanium ethoxide (9.41 g, 41.25 mmol) and benzaldehyde (2.19 g, 20.7 mmol) was heated at 70° C. for 1 h, cooled, and diluted with H₂O (250 mL). The aqueous layer was extracted with EtOAc (3×80 mL) and the combined organic layers were washed with brine (2×100 mL), dried with Na₂SO₄, filtered and concentrated under reduced pressure to afford the desired product (4.3 g, crude) which was used without further purification. LCMS (ESI) m/z: [M+H] calcd for C₁₁H₁₅NOS: 210.10; found 210.2.

### Step 2: Synthesis of ethyl (2S,3S)-1-((S)-tert-butylsulfinyl)-3-phenylaziridine-2-carboxylate

To a solution of ethyl bromoacetate (798 mg, 4.78 mmol) in THF (15 mL) at −78° C. was added LiHMDS (1M in THF, 4.78 mL, 4.78 mmol). After 1 h, (S,E)-N-benzylidene-2-methylpropane-2-sulfinamide (500 mg, 2.39 mmol) in THF (5 mL) was added in portions over 20 min. The reaction mixture was stirred at −78° C. for 2 h and then quenched by the addition of sat. NH₄Cl. The aqueous layer was extracted with EtOAc (3×40 mL) and the combined organic layers were washed with brine (2×30 mL), dried with Na₂SO₄, filtered, and concentrated under reduced pressure. Purification by reverse phase chromatography (30→60% MeCN/H₂O, 0.1% HCO₂H) afforded the desired product (480 mg, 61% yield). LCMS (ESI) m/z: [M+H] calcd for C₁₅H₂₁NO₃S: 296.13; found 296.2.

### Step 3: Synthesis (2S,3S)-1-((S)-tert-butylsulfinyl)-3-phenylaziridine-2-carboxylic acid

To a solution of ethyl (2S,3S)-1-((S)-tert-butylsulfinyl)-3-phenylaziridine-2-carboxylate (600 mg, 2.03 mmol) in THF (4.0 mL) at 0° C. was added a solution of LiOH (97.2 mg, 4.06 mmol) in H₂O (4.0 mL). The resulting mixture was stirred for 2 h at 0° C. and then acidified to pH 5 with 1 M HCl. The aqueous layer was extracted with EtOAc (3×40 mL) and the combined organic layers were washed with brine (2×20 mL), dried with Na₂SO₄, filtered, and concentrated under reduced pressure to afford the desired compound (450 mg, crude) which was used without further purification. LCMS (ESI) m/z: [M+H] calcd for C₁₃H₁₇NO₃S: 268.10; found 268.1.

### Intermediate A-44. Synthesis of (2R,3R)-1-((R)-tert-butylsulfinyl)-3-phenylaziridine-2-carboxylic acid

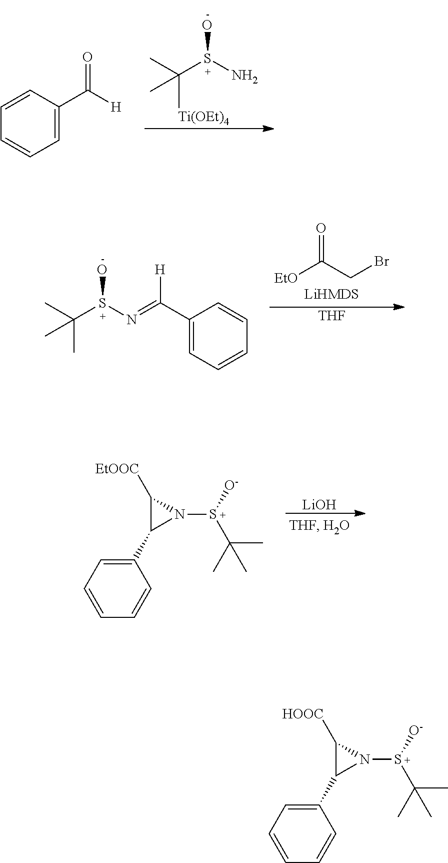

### Step 1: Synthesis (R,E)-N-benzylidene-2-methylpropane-2-sulfinamide

A solution (R)-2-methylpropane-2-sulfinamide (2.50 g, 20.6 mmol), titanium tetraethoxide (9.41 g, 41.3 mmol) and benzaldehyde (2.19 g, 20.6 mmol) was heated 70° C. for 1 h, cooled, and diluted with H₂O (250 mL). The aqueous layer was extracted with EtOAc (3×90 mL) and the combined organic layers were washed with brine (2×100 mL), dried with Na₂SO₄, filtered and concentrated under reduced pressure to afford the desired product (4.2 g, crude) which was used without further purification. LCMS (ESI) m/z: [M+H] calcd for C₁₁H₁₅NOS: 210.10; found 210.1.

### Step 2: Synthesis of ethyl (2R,3R)-1-((R)-tert-butylsulfinyl)-3-phenylaziridine-2-carboxylate

To a solution of ethyl bromoacetate (6.38 g, 38.2 mmol) in THF (150 mL) at −78° C. was added LiHMDS (1M in THF, 7.19 mL, 42.9 mmol). After 1 h, (R,E)-N-benzylidene-2-methylpropane-2-sulfinamide (4.0 g, 19.1 mmol) in THF (50 mL) was added in portions over 20 min. The reaction mixture was stirred at −78° C. for 2 h and then quenched by the addition of sat. NH₄Cl. The aqueous layer was extracted with EtOAc (3×80 mL) and the combined organic layers were washed with brine (2×60 mL), dried with Na₂SO₄, filtered and concentrated under reduced pressure. Purification by reverse phase chromatography (30→60% MeCN/H₂O, 0.1/% HCO₂H) afforded the desired product (3.9 g, 62% yield). LCMS (ESI) m/z: [M+H] calcd for C₁₅H₂₁NO₃S: 296.13; found 296.2.

### Step 3: Synthesis (2R,3R)-1-((R)-tert-butylsulfinyl)-3-phenylaziridine-2-carboxylic acid

To a solution of ethyl (2R,3R)-1-((R)-tert-butylsulfinyl)-3-phenylaziridine-2-carboxylate (200 mg, 0.677 mmol) in THF (1.5 mL) at 0° C. was added a solution of LiOH (32.4 mg, 1.35 mmol) in H₂O (1.3 mL). The resulting mixture was stirred for 2 h at 0° C. and then acidified to pH 5 with 1M HCl. The aqueous layer was extracted with EtOAc (3×20 mL) and the combined organic layers were washed with brine (2×10 mL), dried with Na₂SO₄, filtered, and concentrated under reduced pressure to afford the desired compound (220 mg, crude) which was used without further purification. LCMS (ESI) m/z: [M+H] calcd for C₁₃H₁₇NO₃S: 268.10; found 268.4.

### Intermediate A-45 and A-46. Synthesis of and (S)—N-(1-(2-methoxyethyl)aziridine-2-carbonyl)-N-methylglycine and (R)—N-(1-(2-methoxyethyl)aziridine-2-carbonyl)-N-methylglycine

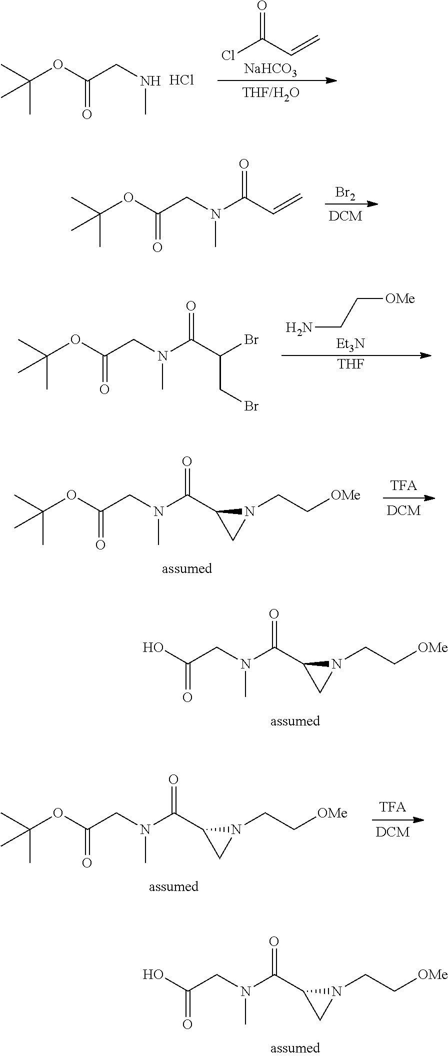

### Step 1: Synthesis of tert-butyl N-acryloyl-N-methylglycinate

To a mixture of tert-butyl methylglycinate hydrochloride (1.0 g, 5.5 mmol) and NaHCO₃ (1.39 g, 16.5 mmol) in THF (10 mL) and H₂O (5.0 mL) at 0° C. was added acryloyl chloride (750 mg, 8.26 mmol). The resulting solution was stirred for 2 h at room temperature and the reaction was then quenched by the addition H₂O (50 mL). The aqueous layer was extracted with EtOAc (2×50 mL) and the combined organic layers were washed with brine, dried with Na₂SO₄, filtered, and concentrated under reduced pressure. Purification by normal phase chromatography (10→33% EtOAc/pet. ether) afforded the desired product (900 mg, 73.8% yield). LCMS (ESI) m/z: [M+H] calcd for C₁₀H₁₇NO₃: 200.13; found 200.2.

### Step 2: Synthesis of tert-butyl N-(2,3-dibromopropanoyl)-N-methylglycinate

To a solution of tert-butyl N-acryloyl-N-methylglycinate (2.0 g, 10.1 mmol) in DCM (40 mL) at −20° C. was added Br₂ (3.21 g, 20.1 mmol). The resulting mixture was stirred for 2 h at −20° C. and then quenched by the addition of Na₂S₂O₃ (100 mL). The aqueous layer was extracted with DCM (2×100 mL) and the combined organic layers were washed with brine, dried with Na₂SO₄, and concentrated under reduced pressure to afford the desired product (2.4 g, crude) which was used without further purification. LCMS (ESI) m/z: [M+Na] calcd for C₁₀H₁₇Br₂NO₃: 381.96; found 381.8.

### Step 3: Synthesis of tert-butyl (S)—N-(1-(2-methoxyethyl)aziridine-2-carbonyl)-N-methylglycinate and tert-butyl (R)—N-(1-(2-methoxyethyl)aziridine-2-carbonyl)-N-methylglycinate

To a solution of tert-butyl N-(2,3-dibromopropanoyl)-N-methylglycinate (4.0 g, 11.1 mmol) and 2-methoxyethan-1-amine (4.18 g, 55.7 mmol) in THF (40 mL) was added Et₃N (4.66 mL, 33.4 mmol). The resulting solution was stirred at 35° C. overnight was then quenched by the addition of H₂O. The aqueous layer was extracted with DCM (2×100 mL) and the combined organic layers were washed with brine, dried with Na₂SO₄, filtered, and concentrated under reduced pressure. Purification by reverse phase chromatography (30→50% MeCN/H₂O) afforded a mixture of the desired products. The enantiomers were separated by chiral preparative normal phase chromatography (hexane, 10 mM NH₃-MeOH/EtOH) to afford tert-butyl (S)—N-(1-(2-methoxyethyl)aziridine-2-carbonyl)-N-methylglycinate (400 mg, 33.3% yield) and tert-butyl (R)—N-(1-(2-methoxyethyl)aziridine-2-carbonyl)-N-methylglycinate (360 mg, 30% yield). LCMS (ESI) m/z: [M+H] calcd for C₁₃H₂₄N₂O₄: 273.18; found 273.0.

### Step 4: Synthesis of (S)—N-(1-(2-methoxyethyl)aziridine-2-carbonyl)-N-methylglycine

To a solution of tert-butyl (S)—N-(1-(2-methoxyethyl)aziridine-2-carbonyl)-N-methylglycinate (250 mg, 0.918 mmol) in DCM (6.0 mL) at 0° C. was added TFA (3.0 mL). The resulting mixture was stirred at 2 h at 0° C. and then concentrated under reduced pressure to afford the desired product (250 mg, crude) which was used without further purification. LCMS (ESI) m/z: [M+H] calcd for C₉H₁₆N₂O₄: 217.12; found 217.1.

### Step 5: Synthesis of (R)—N-(1-(2-methoxyethyl)aziridine-2-carbonyl)-N-methylglycine

To a solution tert-butyl (R)—N-(1-(2-methoxyethyl)aziridine-2-carbonyl)-N-methylglycinate (180 mg, 0.661 mmol) in DCM (6.0 mL) at 0° C. was added TFA (3.0 mL). The resulting mixture was stirred at 2 h at 0° C. and then concentrated under reduced pressure to afford the desired product (150 mg, crude) which was used without further purification. LCMS (ESI) m/z: [M+H] calcd for C₉H₁₆N₂O₄: 217.12; found 217.1.

### Intermediate A-47 and A-48. Synthesis of N-methyl-N-(1-(((R)-1-methylaziridin-2-yl)sulfonyl)piperidine-4-carbonyl)-L-valine and N-methyl-N-(1-(((S)-1-methylaziridin-2-yl)sulfonyl)piperidine-4-carbonyl)-L-valine

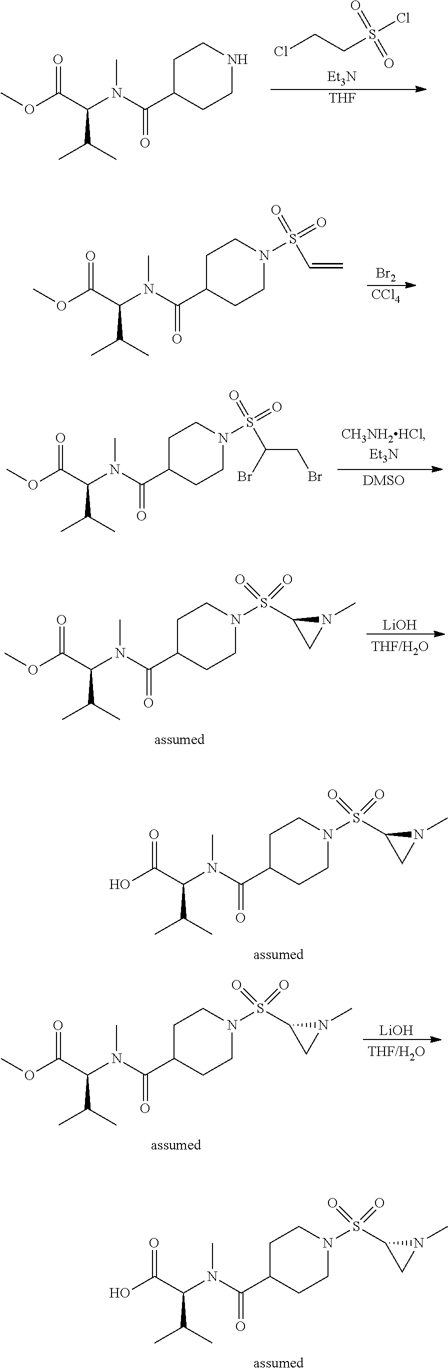

### Step 1: Synthesis of methyl N-methyl-N-(1-(vinylsulfonyl)piperidine-4-carbonyl)-L-valinate

To a solution of 2-chloroethanesulfonyl chloride (1.91 g, 11.7 mmol) in THF (20 mL) at −70° C. was added methyl N-methyl-N-(piperidine-4-carbonyl)-L-valinate (2.0 g, 7.8 mmol) followed by Et₃N (790 L, 780 mol). After warming to −50° C. additional Et₃N (790 L, 780 mol) was added and the reaction mixture warmed to room temperature. After 1 h the reaction was quenched at 0° C. by the addition of H₂O (30 mL), acidified to pH 6 with 1M HCl, and extracted with CHCl₃ (3×30 mL). The combined organic layers were dried with MgSO₄, filtered, and concentrated under reduced pressure. Purification by normal phase chromatography (50% EtOAc/pet. ether) afforded the desired product (560 mg, 20.7% yield). LCMS (ESI) m/z: [M+H] calcd for C₁₅H₂₆N₂O₅S: 347.17; found 347.2.

### Step 2: Synthesis of methyl N-(1-((1,2-dibromoethyl)sulfonyl)piperidine-4-carbonyl)-N-methyl-L-valinate

To a solution of methyl N-methyl-N-(1-(vinylsulfonyl)piperidine-4-carbonyl)-L-valinate (580 mg, 1.67 mmol) in CCl₄ (28 mL) at room temperature was added Br₂ (580 mg, 1.67 mmol) The resulting mixture was stirred overnight at room temperature and then quenched by the addition of sat. NaHCO₃ (30 mL). The aqueous layer was extracted with DCM (3×30 mL) and the combined organic layers were dried with Na₂SO₄, filtered and concentrated under reduced pressure to afford the desired product which was used without further purification. LCMS (ESI) m/z: [M+H] calcd for C₁₅H₂₆Br₂N₂O₅S: 506.99; found 506.9.

### Step 3: Synthesis of methyl N-methyl-N-(1-(((R)-1-methylaziridin-2-yl)sulfonyl)piperidine-4-carbonyl)-L-valinate and methyl N-methyl-N-(1-(((S)-1-methylaziridin-2-yl)sulfonyl)piperidine-4-carbonyl)-L-valinate

To a solution of methyl N-(1-((1,2-dibromoethyl)sulfonyl)piperidine-4-carbonyl)-N-methyl-L-valinate (4.80 g, 9.481 mmol) in DMSO (48 mL) was added methanamine hydrochloride (1.92 g, 28.436 mmol) and Et₃N (13.2 mL, 94.8 mmol). The reaction mixture was heated to 75° C. and stirred overnight. The mixture was then cooled to 0° C., diluted NH₄Cl, and extracted with EtOAc (600 mL). The organic layer was washed with brine, dried with Na₂SO₄, filtered, concentrated under reduced pressure. Purification with normal phase chromatography (86% EtOAc/hexane) afforded a mixture of the desired products. The diastereomers were separated by prep-SFC chromatography (20% IPA/CO₂) to afford methyl N-methyl-N-(1-(((R)-1-methylaziridin-2-yl)sulfonyl)piperidine-4-carbonyl)-L-valinate (700 mg, 38.9% yield) and methyl N-methyl-N-(1-(((S)-1-methylaziridin-2-yl)sulfonyl)piperidine-4-carbonyl)-L-valinate (790 mg, 43.9% yield). LCMS (ESI) m/z: [M+H] calcd for C₁₆H₂₉N₃O₅S: 376.19; found 376.1.

### Step 4: Synthesis of N-methyl-N-(1-(((R)-1-methylaziridin-2-yl)sulfonyl)piperidine-4-carbonyl)-L-valine

To a solution of methyl N-methyl-N-(1-(((R)-1-methylaziridin-2-yl)sulfonyl)piperidine-4-carbonyl)-L-valinate (200 mg, 0.533 mmol) in THF (2.0 mL) at 0° C. was added 1M LiOH (1 mL) The resulting mixture was stirred for 3 h at room temperature and then acidified to pH 6 with 1M HCl. The aqueous layer was extracted with EtOAc (3×10 mL) and the combined organic layers were dried with Na₂SO₄, filtered, and concentrated under reduced pressure to afford the desired product which was used without further purification. LCMS (ESI) m/z: [M+H] calcd for C₁₅H₂₇N₃O₅S: 362.18; found 362.2.

### Step 5: Synthesis of N-methyl-N-(1-(((S)-1-methylaziridin-2-yl)sulfonyl)piperidine-4-carbonyl)-L-valine

To a solution methyl N-methyl-N-(1-(((S)-1-methylaziridin-2-yl)sulfonyl)piperidine-4-carbonyl)-L-valinate (300 mg, 0.799 mmol) in THF (3.0 mL) at 0° C. was added 1M LiOH (3.0 mL). The resulting mixture was stirred for 3 h at room temperature and then acidified to pH 6 with 1M HCl. The aqueous layer was extracted with EtOAc (3×10 mL) and the combined organic layers dried with Na₂SO₄, filtered and concentrated under reduced pressure to afford the desired product which was used without further purification. LCMS (ESI) m/z: [M+H] calcd for C₁₅H₂₇N₃O₅S: 362.18; found 362.2.

### Intermediate A-49 and A-50. Synthesis of N-methyl-N-(1-(((R)-1-methylaziridin-2-yl)sulfonyl)azetidine-3-carbonyl)-L-valine and N-methyl-N-(1-(((S)-1-methylaziridin-2-yl)sulfonyl)azetidine-3-carbonyl)-L-valine

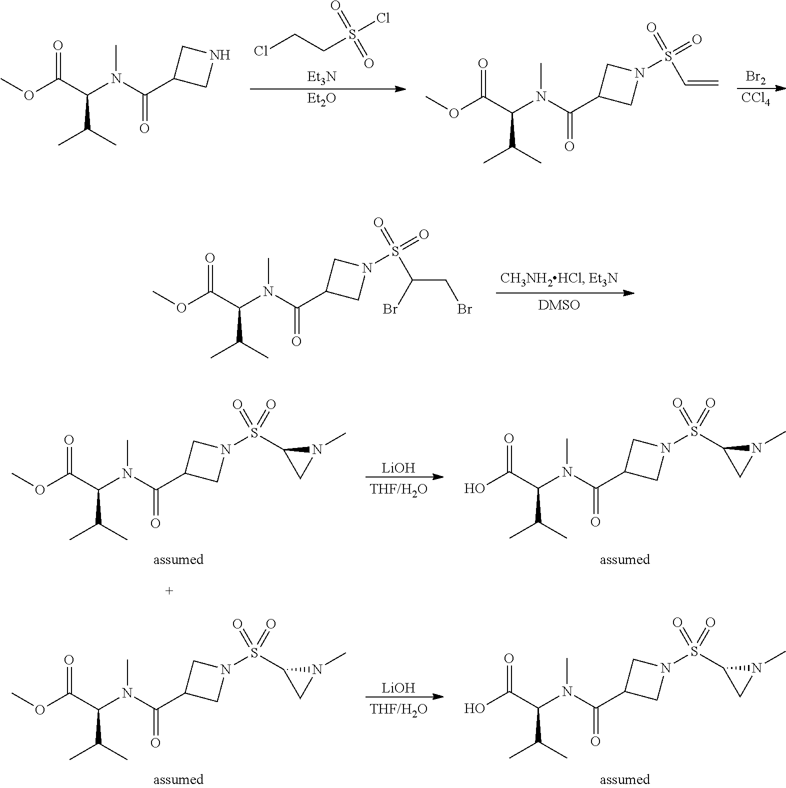

### Step 1: Synthesis of methyl N-methyl-N-(1-(vinylsulfonyl)azetidine-3-carbonyl)-L-valinate

To a solution of 2-chloroethanesulfonyl chloride (357 mg, 2.19 mmol) in Et₂O (4.0 mL) at −70° C. was added an Et₂O (4.0 mL) solution of methyl N-(azetidine-3-carbonyl)-N-methyl-L-valinate (500 mg, 2.19 mmol) followed by Et₃N (0.304 mL, 2.19 mmol). The resulting mixture was stirred for 30 min at −50° C. at which time Et₃N (0.304 mL, 2.19 mmol) was added. The resulting mixture was stirred for 1 h at room temperature and then quenched with H₂O at 0° C. The mixture was acidified to pH 6 with 1M HCl and extracted with CHCl₃ (3×10 mL). The combined organic layers were washed with brine, dried with Na₂SO₄, filtered, and concentrated under reduced pressure. Purification by normal phase chromatography (50% EtOAc/pet. ether) afforded the desired product (180 mg, 25.8% yield). LCMS (ESI) m/z: [M+H] calcd for C₁₃H₂₂N₂O₅S: 319.13; found 319.1.

### Step 2: Synthesis of methyl N-(1-((1,2-dibromoethyl)sulfonyl)azetidine-3-carbonyl)-N-methyl-L-valinate

To a solution of methyl N-methyl-N-(1-(vinylsulfonyl)azetidine-3-carbonyl)-L-valinate (460 mg, 1.45 mmol) in CCl₄ (6.0 mL) at room temperature was added a CCl₄ (2.0 mL) solution of Br₂ (346 mg, 2.17 mmol). The resulting mixture was stirred overnight and then quenched at 0° C. by the addition of sat. NaHCO₃ and Na₂S₂O₃. The aqueous layer was extracted with DCM (3×10 mL) and the combined organic layers were washed with brine, dried with Na₂SO₄, and concentrated under reduced pressure to afford the desired product (500 mg) which was used without further purification. LCMS (ESI) m/z: [M+H] calcd for C₁₃H₂₂Br₂N₂O₅S: 478.97; found 478.0.

### Step 3: Synthesis of methyl N-methyl-N-(1-(((R)-1-methylaziridin-2-yl)sulfonyl)azetidine-3-carbonyl)-L-valinate and methyl N-methyl-N-(1-(((S)-1-methylaziridin-2-yl)sulfonyl)azetidine-3-carbonyl)-L-valinate

To a solution of methyl N-(1-((1,2-dibromoethyl)sulfonyl)azetidine-3-carbonyl)-N-methyl-L-valinate (260 mg, 0.54 mmol) in DMSO (4.0 mL) was added methanamine hydrochloride (110.0 mg, 1.63 mmol) and Et₃N (0.758 mL, 5.44 mmol). The resulting mixture was heated to 75° C. and stirred overnight. The mixture was then cooled to room temperature, diluted with H₂O (10 mL), and extracted with EtOAc (3×5 mL). The combined organic layers were washed with brine, dried with Na₂SO₄, filtered and concentrated under reduced pressure. Purification by normal phase chromatography (50% EtOAc/pet. ether) afforded a mixture of the desired products. The diastereomers were separated by chiral prep normal phase chromatography (hexane, 10 mM NH₃-MeOH/IPA) to afford methyl N-methyl-N-(1-(((R)-1-methylaziridin-2-yl)sulfonyl)azetidine-3-carbonyl)-L-valinate (0.59 g, 35% yield) and methyl N-methyl-N-(1-(((S)-1-methylaziridin-2-yl)sulfonyl)azetidine-3-carbonyl)-L-valinate (0.56 g, 33% yield). LCMS (ESI) m/z: [M+H] calcd for C₁₄H₂₅N₃O₅S: 348.16; found 348.2.

### Step 4: Synthesis of N-methyl-N-(1-(((R)-1-methylaziridin-2-yl)sulfonyl)azetidine-3-carbonyl)-L-valine

To a solution of methyl N-methyl-N-(1-(((R)-1-methylaziridin-2-yl)sulfonyl)azetidine-3-carbonyl)-L-valinate (225.0 mg, 0.65 mmol) in THF (1.5 mL) at 0° C. was added LiOH (77.0 mg, 3.23 mmol) dissolved in H₂O (1.5 mL). The resulting mixture was stirred for 2 h at room temperature and then acidified to pH 6 with 1M HCl. The aqueous layer was extracted with EtOAc and the combined organic layers were washed with brine, dried with Na₂SO₄, filtered, and concentrated under reduced pressure to afford the desired product (270 mg) which was used without further purification. LCMS (ESI) m/z: [M+H] calcd for C₁₃H₂₃N₃O₅S: 334.15; found 334.0.

### Step 5: Synthesis of N-methyl-N-(1-(((S)-1-methylaziridin-2-yl)sulfonyl)azetidine-3-carbonyl)-L-valine

To a solution of methyl N-methyl-N-(1-(((S)-1-methylaziridin-2-yl)sulfonyl)azetidine-3-carbonyl)-L-valinate (365.0 mg, 1.05 mmol) in THF (2.0 mL) and H₂O (2.0 mL) at 0° C. was added LiOH hydrate (132.0 mg, 3.15 mmol). The resulting mixture was stirred for 2 h at room temperature then acidified to pH 6 with 1M HCl and diluted with H₂O (20 mL). The aqueous layer was extracted with EtOAc and the combined organic layers were washed with brine, dried with Na₂SO₄, filtered, and concentrated under reduced pressure to afford the desired product (257 mg, crude) which was used without further purification. LCMS (ESI) m/z: [M+H] calcd for C₁₃H₂₃N₃O₅S: 334.15; found 334.3.

### Intermediate A-51. Synthesis of 2-((1R,5S)-2,4-dioxo-6-trityl-3,6-diazabicyclo[3.1.0]hexan-3-yl)acetic acid

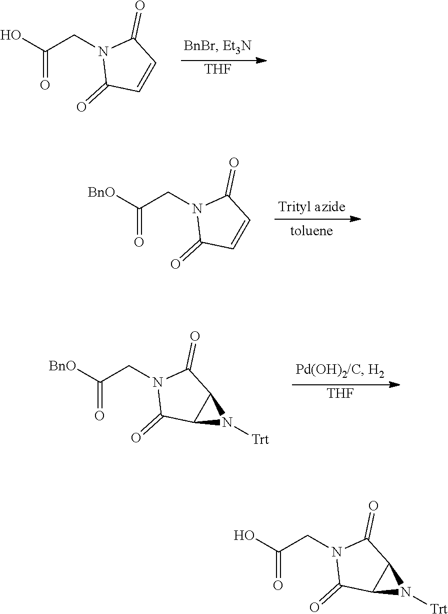

### Step 1: Synthesis of benzyl 2-(2,5-dioxo-2,5-dihydro-1H-pyrrol-1-yl)acetate

To a solution of 2-(2,5-dioxo-2,5-dihydro-1H-pyrrol-1-yl)acetic acid (5.0 g, 32.2 mmol) and Et₃N (13.5 mL, 96.7 mmol) in THF (80 mL) at 0° C. was added benzyl bromide (11.03 g, 64.5 mmol). The resulting mixture was stirred overnight at room temperature and then filtered. The filter cake was washed with THF (3×40 mL) and the filtrate was concentrated under reduced pressure. Purification by silica gel chromatography (16% EtOAc/hexanes) afforded the desired product (4.4 g, 55.7% yield) LCMS (ESI) m/z: [2M+Na] calcd for C₁₃H₁₁NO₄: 513.14; found 513.2.

### Step 2: Synthesis of benzyl 2-((1R,5S)-2,4-dioxo-6-trityl-3,6-diazabicyclo[3.1.0]hexan-3-yl)acetate

To a solution of benzyl 2-(2,5-dioxo-2,5-dihydro-1H-pyrrol-1-yl)acetate of (1.0 g, 4.0 mmol) in toluene (10 mL) was added trityl azide (1.36 g, 4.89 mmol). The resulting mixture was stirred overnight at 120° C. and then concentrated under reduced pressure. Purification by reverse flash chromatography (50→80% MeCN/H₂O) afforded the desired product (400 mg, 19.5% yield). LCMS (ESI) m/z: [M+Na] calcd for C₃₂H₂₆N₂O₄: 525.19; found 525.2.

### Step 3: Synthesis of 2-((1R,5S)-2,4-dioxo-6-trityl-3,6-diazabicyclo[3.1.0]hexan-3-yl)acetic acid

To a solution of benzyl 2-((1R,5S)-2,4-dioxo-6-trityl-3,6-diazabicyclo[3.1.0]hexan-3-yl)acetate (220 mg, 0.438 mmol) in THF (8.0 mL) was added Pd(OH)₂/C (60 mg). The resulting solution was placed under a hydrogen atmosphere for 3 h using a H₂ balloon, filtered through Celite, and concentrated under reduced pressure to afford the desired product (160 mg, crude) which was used without further purification. LCMS (ESI) m/z: [M−H] calcd for C₂₅H₂₀N₂O₄: 411.13; found 411.2.

### Intermediate A-52. Synthesis of (S)-3-methyl-2-(5-oxo-2-((S)-1-tritylaziridine-2-carbonyl)-2,6-diazaspiro[3.4]octan-6-yl)butanoic acid

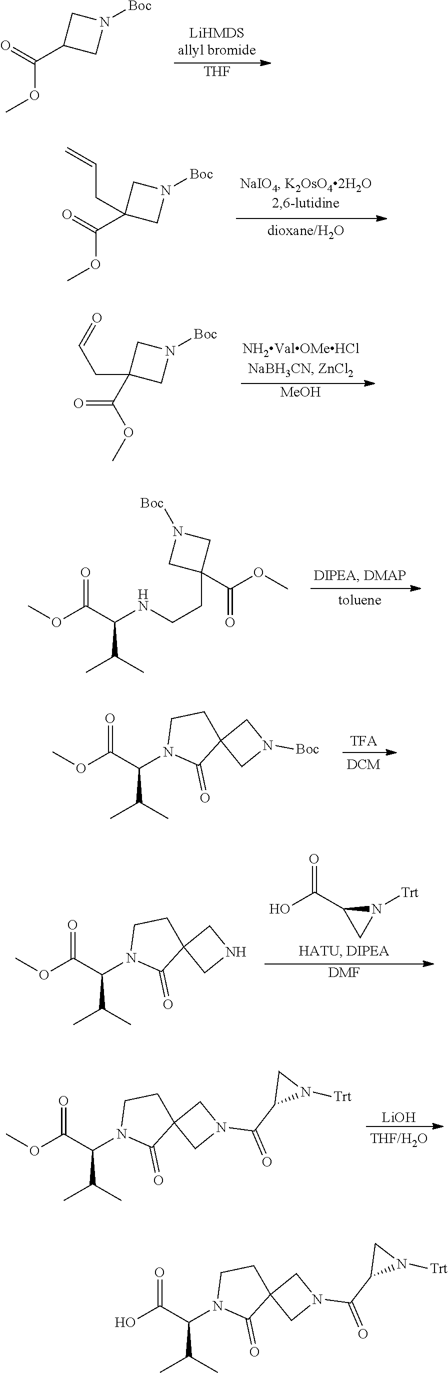

### Step 1: Synthesis of 1-(tert-butyl) 3-methyl 3-allylazetidine-1,3-dicarboxylate

To a solution of 1-(tert-butyl) 3-methyl azetidine-1,3-dicarboxylate (20.0 g, 92.9 mmol) and LiHMDS (140 mL, 1M in THF, 139 mmol) in THF (200 mL) at −78° C. was added allyl bromide (16.9 g, 139 mmol). The resulting solution was stirred overnight at room temperature and then quenched with the addition of sat. NH₄Cl (100 mL) and diluted with EtOAc (800 mL). The organic layer was washed with brine (3×300 mL), dried with Na₂SO₄, filtered and concentrated under reduced pressure. Purification by silica gel column chromatography (17% EtOAc/pet. ether) afforded the desired product (15.0 g, 63.2% yield). LCMS (ESI) m/z: [M+H−Bu] calcd for C₁₃H₂₁NO₄: 200.10; found 200.0.

### Step 2: Synthesis of 1-(tert-butyl) 3-methyl 3-(2-oxoethyl)azetidine-1,3-dicarboxylate

To a solution of 1-(tert-butyl) 3-methyl 3-allylazetidine-1,3-dicarboxylate (6.0 g, 23 mmol) and 2,6-lutidine (504 mg, 47.0 mmol) in dioxane (60 mL) and H₂O (60 mL) at 0° C. was added K₂OsO₄·2H₂O (433 mg, 1.18 mmol). The resulting mixture was stirred at room temperature for 15 min then NaIO₄ (20.1 g, 94.0 mmol) was added at 0° C. The reaction was stirred for 3 h at room temperature and then quenched with sat. Na₂S₂O₃ at 0° C. The aqueous layer was extracted with EtOAc (2×400 mL) and the combined organic layers were washed with 1 M HCl (2×80 mL), brine (2×100 mL), dried with Na₂SO₄, filtered and concentrated under reduced pressure to afford the desired product (2.84 g, crude) which was used without further purification. LCMS (ESI) m/z: [M−H] calcd for C₁₂H₁₉NO₅: 256.12; found 256.0.

### Step 3: Synthesis of 1-(tert-butyl) 3-methyl (S)-3-(2-((1-methoxy-3-methyl-1-oxobutan-2-yl)amino)ethyl)azetidine-1,3-dicarboxylate

To a solution of 1-(tert-butyl) 3-methyl 3-(2-oxoethyl)azetidine-1,3-dicarboxylate (13.0 g, 50.5 mmol) and methyl L-valinate hydrochloride (7.29 g, 55.6 mmol) in MeOH (130 mL) at 0° C. were added ZnCl₂ (7.57 g, 55.6 mmol) and NaBH₃CN (6.35 g, 101 mmol). The resulting mixture was stirred at room temperature overnight, partially concentrated under reduced pressure and diluted with EtOAc (500 mL). The resulting solution was washed with brine (3×200 mL), dried with Na₂SO₄, filtered, and concentrated under reduced pressure. Purification by normal phase chromatography (10→66% EtOAc/pet. ether) afforded the desired product (7.72 g, 41.0% yield). LCMS (ESI) m/z: [M+H] calcd for C₁₈H₃₂N₂O₆: 373.24; found 373.1.

### Step 4: Synthesis of tert-butyl (S)-6-(1-methoxy-3-methyl-1-oxobutan-2-yl)-5-oxo-2,6-diazaspiro[3.4]octane-2-carboxylate

To a solution of 1-(tert-butyl) 3-methyl (S)-3-(2-((1-methoxy-3-methyl-1-oxobutan-2-yl)amino)ethyl)azetidine-1,3-dicarboxylate (6.0 g, 16 mmol) and DIPEA (28.0 mL, 161 mmol) in toluene (60 mL) at room temperature was added DMAP (197 mg, 1.61 mmol). The resulting mixture was stirred at 80° C. overnight, diluted with EtOAc (50 mL), washed with H₂O (50 mL), brine (3×50 mL) dried with Na₂SO₄, and filtered, and concentrated under reduced pressure. Purification by reverse phase chromatography 45-80% MeCN/H₂O) afforded the desired product (4.3 g, 78.4% yield). LCMS (ESI) m/z: [M+H−tBu] calcd for C₁₇H₂₈N₂O₅: 285.15; found 285.0.

### Step 5: Synthesis of methyl (S)-3-methyl-2-(5-oxo-2,6-diazaspiro[3.4]octan-6-yl)butanoate

To a solution of tert-butyl (S)-6-(1-methoxy-3-methyl-1-oxobutan-2-yl)-5-oxo-2,6-diazaspiro[3.4]octane-2-carboxylate (2.7 g, 7.9 mmol) in DCM (27 mL) at room temperature was added TFA (8.10 mL, 71.0 mmol). The resulting mixture was stirred for 1 h and then concentrated under reduced pressure to afford the desired product, (1.70 g, crude) which was used without further purification. LCMS (ESI) m/z: [M+H] calcd for C₁₂H₂₀N₂O₃: 241.16; found 240.1.

### Step 6: Synthesis of methyl (S)-3-methyl-2-(5-oxo-2-((S)-1-tritylaziridine-2-carbonyl)-2,6-diazaspiro[3.4]octan-6-yl)butanoate

To a solution methyl (S)-3-methyl-2-(5-oxo-2,6-diazaspiro[3.4]octan-6-yl)butanoate (700 mg, 2.91 mmol) and (S)-1-tritylaziridine-2-carboxylic acid (1.15 g, 3.50 mmol) in DMF (7.0 mL) at 0° C. was added DIPEA (2.5 mL, 14.6 mmol). After 30 min HATU (1.66 g, 4.37 mmol) was added and the resulting mixture was stirred at room temperature for 1 h. The reaction was then diluted with EtOAc (20 mL) and the organic layer was washed with sat. NH₄Cl (50 mL), brine (3×50 mL), dried with Na₂SO₄, filtered, and concentrated under reduced pressure. Purification by normal phase chromatography (0→80% EtOAc/pet. ether) afforded the desired product (300 mg, 18.7% yield). LCMS (ESI) m/z: [M+H] calcd for C₃₄H₃₇N₃O₄: 552.29; found 552.2.

### Step 7: Synthesis of (S)-3-methyl-2-(5-oxo-2-((S)-1-tritylaziridine-2-carbonyl)-2,6-diazaspiro[3.4]octan-6-yl)butanoic acid

To a solution of methyl (S)-3-methyl-2-(5-oxo-2-((S)-1-tritylaziridine-2-carbonyl)-2,6-diazaspiro[3.4]octan-6-yl)butanoate (700 mg, 1.27 mmol) in THF (10 mL) and H₂O (2.0 mL) at 0° C. was added LiOH (152 mg, 6.34 mmol). After 30 min the reaction mixture was warmed to room temperature for 1 h and then acidified to pH 6 with 1M HCl. The aqueous layer was extracted with EtOAc (3×50 mL) and the combined organic layers were washed with brine, dried with Na₂SO₄, filtered, and concentrated under reduced pressure to afford the desired product (300 mg, 18.7% yield) which was used without further purification. LCMS (ESI) m/z: [M+H] calcd for C₃₃H₃₅N₃O₄: 538.27; found 538.2.

### Intermediate A-53. Synthesis of (S)-3-methyl-2-(5-oxo-2-((R)-1-tritylaziridine-2-carbonyl)-2,6-diazaspiro[3.4]octan-6-yl)butanoic acid

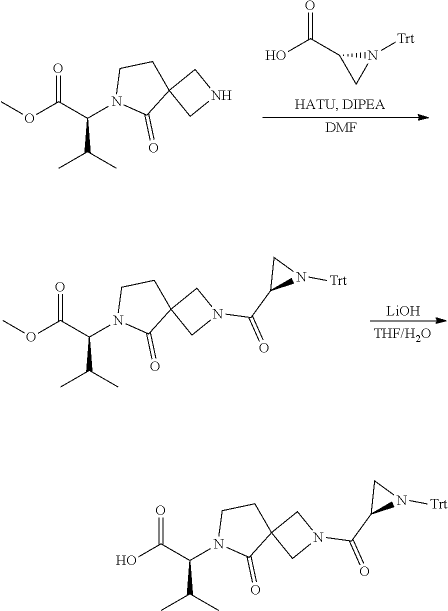

### Step 1: Synthesis of methyl (S)-3-methyl-2-(5-oxo-2-((R)-1-tritylaziridine-2-carbonyl)-2,6-diazaspiro[3.4]octan-6-yl)butanoate

To a solution (R)-1-tritylaziridine-2-carboxylic acid (1.0 g, 3.0 mmol) and methyl (S)-3-methyl-2-(5-oxo-2,6-diazaspiro[3.4]octan-6-yl)butanoate (875 mg, 3.64 mmol) in DMF (10 mL) at 0° C. was added DIPEA (2.64 mL, 15.2 mmol). After 30 min HATU (1.73 g, 4.554 mmol) was added and the resulting mixture was stirred for 1 h at room temperature. The reaction was then diluted with EtOAc (20 mL) and the organic layer was washed with sat. NH₄Cl (50 mL), brine (3×50 mL), dried with Na₂SO₄, filtered, and concentrated under reduced pressure. Purification by silica gel chromatography (0→80% EtOAc/pet. ether) afforded the desired product (789 mg, 47% yield). LCMS (ESI) m/z: [M+H] calcd for C₃₄H₃₇N₃O₄: 552.29; found 552.3.

### Step 2: Synthesis of (S)-3-methyl-2-(5-oxo-2-((R)-1-tritylaziridine-2-carbonyl)-2,6-diazaspiro[3.4]octan-6-yl)butanoic acid

To a stirred solution of methyl (S)-3-methyl-2-(5-oxo-2-((R)-1-tritylaziridine-2-carbonyl)-2,6-diazaspiro[3.4]octan-6-yl)butanoate (900 mg, 1.63 mmol) in THF (10 mL) and H₂O (2.5 mL) at 0° C. was added LiOH (156 mg, 6.53 mmol). After 30 min the reaction mixture was warmed to room temperature for 1 h and then acidified to pH 6 with 1M HCl. The aqueous layer was extracted with EtOAc (3×50 mL) and the combined organic layers were washed with brine, dried with Na₂SO₄, filtered, and concentrated under reduced pressure to afford the desired product (240 mg, 27.4% yield). LCMS (ESI) m/z: [M+H] calcd for C₃₃H₃₅N₃O₄: 538.27; found 538.2.

### Intermediate A-54. Synthesis of (S)-1-((R)-2-(methoxycarbonyl)aziridine-1-carbonyl)pyrrolidine-3-carboxylic acid

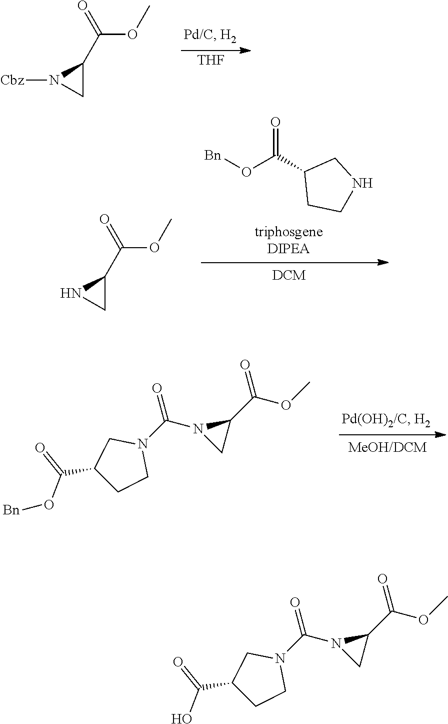

### Step 1: Synthesis of methyl (R)-aziridine-2-carboxylate

A suspension of 1-benzyl 2-methyl (R)-aziridine-1,2-dicarboxylate (1.50 g, 6.4 mmol) and Pd/C (300 mg, 2.8 mmol) in THF (15 mL) under an atmosphere of hydrogen (1 atm) was stirred for 3 h before the solids were removed by filtration. The crude solution was concentrated under reduced pressure which afforded desired product (600 mg, crude). LCMS (ESI) m/z: [M+H] calcd for C₄H₇NO₂: 102.06; found 102.3.

### Step 2: Synthesis of benzyl (S)-1-((R)-2-(methoxycarbonyl)aziridine-1-carbonyl)pyrrolidine-3-carboxylate

To solution of methyl (R)-aziridine-2-carboxylate (1.0 g, 9.90 mmol) and benzyl (S)-pyrrolidine-3-carboxylate (2.63 g, 10.9 mmol, HCl salt) in DCM (30.0 mL) at −10° C. was added DIPEA (10.3 mL, 59.3 mmol) followed by triphosgene (880 mg, 2.97 mmol). The resulting solution was stirred for 30 min and was then quenched by the addition of H₂O (50 mL). The aqueous layer was extracted with DCM (2×100 mL), washed with brine (2×50 mL), dried over Na₂SO₄, and concentrated under reduced pressure. Purification by prep-TLC (50% EtOAc/pet. ether) afforded desired product (1.30 g, 28% yield). LCMS (ESI) m/z: [M+H] calcd for C₁₇H₂₀N₂O₅: 333.15; found 333.2.

### Step 3: Synthesis of (S)-1-((R)-2-(methoxycarbonyl)aziridine-1-carbonyl)pyrrolidine-3-carboxylic acid

To a solution of benzyl (S)-1-((R)-2-(methoxycarbonyl)aziridine-1-carbonyl)pyrrolidine-3-carboxylate (200 mg, 600 μmol) in MeOH (5 mL) and DCM (5 mL) under H₂ was added Pd(OH)₂/C (130 mg, 90 μmol). The resulting mixture was stirred for 30 min at room temperature and then the mixture was filtered. The filter cake was washed with MeOH (2×10 mL) and the filtrate was concentrated under reduced pressure which afforded desired product (140 mg, 96% yield) as an off-white solid. LCMS (ESI) m/z: [M+H] calcd for C₁₀H₁₄N₂O₅: 243.10; found 243.3.

### Intermediate A-55. Synthesis of (S)-1-((S)-2-(methoxycarbonyl)aziridine-1-carbonyl)pyrrolidine-3-carboxylic acid

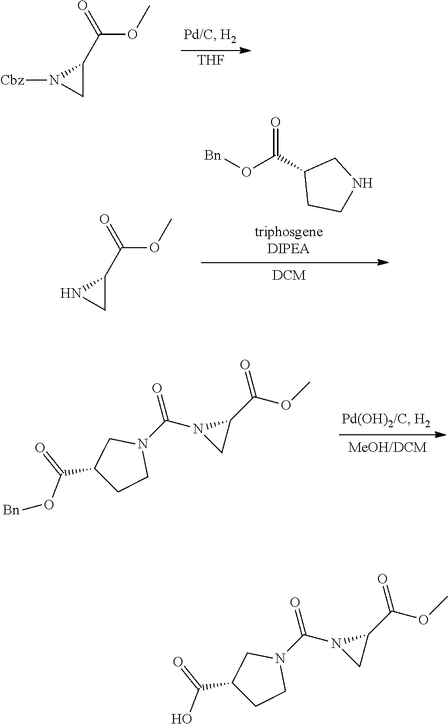

### Step 1: Synthesis of methyl (S)-aziridine-2-carboxylate

A suspension of 1-benzyl 2-methyl (S)-aziridine-1,2-dicarboxylate (200 mg, 850 μmol) and Pd/C (20 mg, 38 μmol) in THF (4.0 mL) under an atmosphere of hydrogen (1 atm) was stirred for 2 h before the solids were removed by filtration. The crude solution was concentrated under reduced pressure which afforded desired product (92 mg, crude). LCMS (ESI) m/z: [M+H] calcd for C₄H₇NO₂: 102.06; found 102.3.

### Step 2: Synthesis of benzyl (S)-1-((S)-2-(methoxycarbonyl)aziridine-1-carbonyl)pyrrolidine-3-carboxylate

To a solution of methyl (S)-aziridine-2-carboxylate (900 mg, 8.9 mmol) and benzyl (S)-pyrrolidine-3-carboxylate (2.37 g, 9.80 mmol, HCl salt) in DCM (30 mL) at −10° C. was added DIPEA (9.30 mL, 53.4 mmol) followed by triphosgene (790 mg, 2.67 mmol). The resulting solution was stirred for 30 min and was then quenched by the addition of H₂O (50 mL). The aqueous layer was extracted with DCM (2×100 mL), washed with brine (2×50 mL), dried over Na₂SO₄, filtered, and concentrated under reduced pressure. Purification by prep-TLC (50% EtOAc/pet. ether) afforded desired product (360 mg, 8.5% yield) as an off-white oil. LCMS (ESI) m/z: [M+H] calcd for C₁₇H₂₀N₂O₅: 333.15; found 333.2.

### Step 3: Synthesis of (S)-1-((S)-2-(methoxycarbonyl)aziridine-1-carbonyl)pyrrolidine-3-carboxylic acid

To a solution of benzyl (S)-1-((S)-2-(methoxycarbonyl)aziridine-1-carbonyl)pyrrolidine-3-carboxylate (130 mg, 390 μmol) in MeOH (3 mL) and DCM (3 mL) under H₂ was added Pd(OH)₂/C (55 mg, 39 μmol). The resulting solution was stirred for 30 min at room temperature and then the reaction mixture was filtered. The filter cake was washed with MeOH (2×10 mL) and the filtrate was concentrated under reduced pressure which afforded desired product (90 mg, 95% yield) as an off-white solid. LCMS (ESI) m/z: [M+H] calcd for C₁₀H₁₄N₂O₅: 243.10; found 243.3.

### Intermediate A-56. Synthesis of (2R,3S)-3-cyclopropylaziridine-2-carboxylic acid

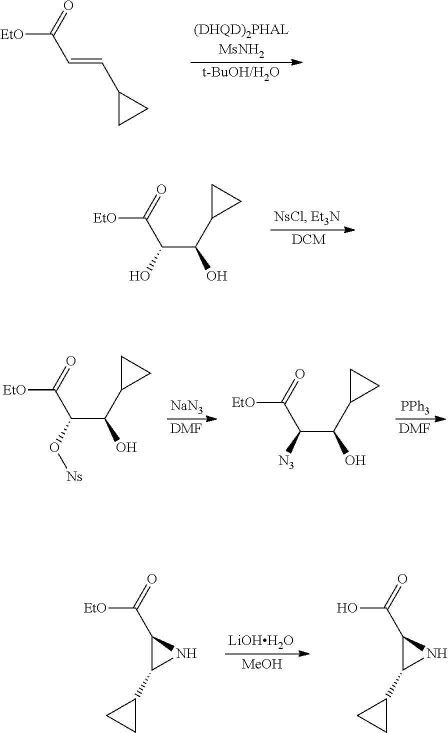

### Step 1: Synthesis of ethyl (2S,3R)-3-cyclopropyl-2,3-dihydroxypropanoate

A solution of ethyl (E)-3-cyclopropylacrylate (10.4 mL, 71 mmol) in tert-BuOH (270 mL) and H₂O (270 mL) was stirred at 0° C. After 5 min MsNH₂ (6.8 g, 71 mmol) and (DHQD)₂PHAL (100 g, 130 mmol) were added and the reaction mixture was warmed to room temperature. After stirring overnight, sat. Na₂SO₃ was added and the mixture was stirred for 30 min. The mixture was acidified to pH 6 with KH₂PO₄. Purification by silica gel column chromatography (33% EtOAc/pet. ether) afforded desired product (5.5 g, 44% yield).

### Step 2: Synthesis of ethyl (2S,3R)-3-cyclopropyl-3-hydroxy-2-(((4-nitrophenyl)sulfonyl)oxy)propanoate

A solution of ethyl (2S,3R)-3-cyclopropyl-2,3-dihydroxypropanoate (5.40 g, 31.0 mmol) and Et₃N (13.0 mL, 93.0 mmol) in DCM (20 mL) was stirred at 0° C. and a solution of 4-nitrobenzenesulfonyl chloride (6.53 g, 29.5 mmol) in DCM (10 mL) was added. The reaction mixture was stirred for 1.5 h and was then extracted with DCM (3×200 mL). The combined organic layers were washed with brine (100 mL), dried with Na₂SO₄, filtered, and concentrated under reduced pressure. Purification by silica gel column chromatography (33% EtOAc/pet. ether) afforded desired product (6.9 g, 62% yield).

### Step 3: Synthesis of ethyl (2R,3R)-2-azido-3-cyclopropyl-3-hydroxypropanoate

A mixture of ethyl (2S,3R)-3-cyclopropyl-3-hydroxy-2-(((4-nitrophenyl)sulfonyl)oxy)propanoate (6.90 g, 19.2 mmol) and NaN₃ (6.24 g, 96.0 mmol) in DMF (70.0 mL) was heated to 50° C. The reaction mixture was stirred for 5 h and then extracted with EtOAc (3×200 mL). The combined organic layers were washed with brine (100 mL), dried with Na₂SO₄, filtered, and concentrated under reduced pressure. Purification by silica gel column chromatography (20% EtOAc/pet. ether) afforded desired product (2.8 g, 73% yield).

### Step 4: Synthesis of ethyl (2R,3S)-3-cyclopropylaziridine-2-carboxylate

A mixture of triphenylphosphine (1.84 g, 7.02 mmol) in DMF (5 mL) was stirred at 0° C. After 5 min ethyl (2R,3R)-2-azido-3-cyclopropyl-3-hydroxypropanoate (1.40 g, 7.03 mmol) was added and the reaction was warmed to room temperature. The reaction mixture was heated to 80° C. and stirred for 1 h. The mixture was then cooled to room temperature and extracted with EtOAc (3×50 mL). The combined organic layers were washed with brine (50 mL), dried with Na₂SO₄, filtered, and concentrated under reduced pressure. Purification by silica gel column chromatography (20% EtOAc/pet. ether) afforded the desired product (230 mg, 46% yield). LCMS (ESI) m/z: [M+H] calcd for C₈H₁₃NO₂: 156.10; found 156.2.

### Step 5: Synthesis of lithium (2R,3S)-3-cyclopropylaziridine-2-carboxylate

To a mixture of ethyl (2R,3S)-3-cyclopropylaziridine-2-carboxylate (230 mg, 1.5 mmol) in MeOH (3.0 mL) was added LiOH·H₂O (125 mg, 3.0 mmol). The reaction was stirred for 3 h and then filtered. The filtrate was concentrated under reduced pressure which afforded the desired product (150 mg, crude). LCMS (ESI) m/z: [M+H] calcd for C₆H₉NO₂: 128.07; found 128.2.

### Intermediate A-57. Synthesis of (2R,3S)-3-cyclopropylaziridine-2-carboxylic acid

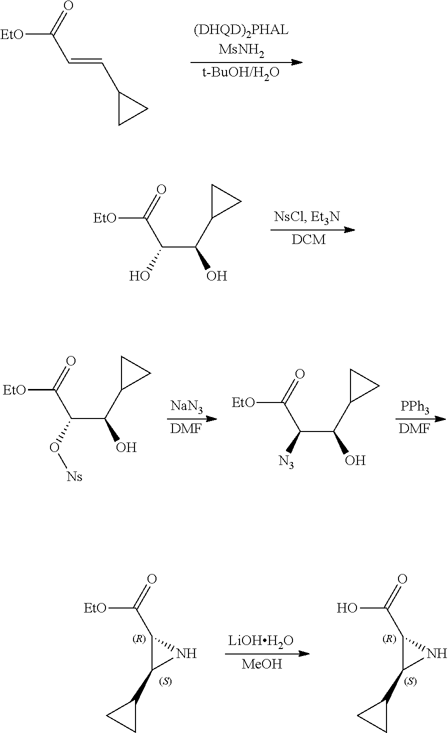

### Step 1: Synthesis of ethyl (2S,3R)-3-cyclopropyl-2,3-dihydroxypropanoate

A solution of ethyl (E)-3-cyclopropylacrylate (10.4 mL, 71 mmol) in tert-BuOH (270 mL) and H₂O (270 mL) was stirred at 0° C. After 5 min MsNH₂ (6.8 g, 71 mmol) and (DHQD)₂PHAL (100 g, 130 mmol) were added and the reaction mixture was warmed to room temperature. After stirring overnight, sat. Na₂SO₃ was added and the mixture was stirred for 30 min. The mixture was acidified to pH 6 with KH₂PO₄. Purification by silica gel column chromatography (33% EtOAC/pet. ether) afforded desired product (5.5 g, 44% yield).

### Step 2: Synthesis of ethyl (2S,3R)-3-cyclopropyl-3-hydroxy-2-(((4-nitrophenyl)sulfonyl)oxy)propanoate

A solution of ethyl (2S,3R)-3-cyclopropyl-2,3-dihydroxypropanoate (5.40 g, 31.0 mmol) and Et₃N (13.0 mL, 93.0 mmol) in DCM (20 mL) was stirred at 0° C. and a solution of 4-nitrobenzenesulfonyl chloride (6.53 g, 29.5 mmol) in DCM (10 mL) was added. The reaction mixture was stirred for 1.5 h and was then extracted with DCM (3×200 mL). The combined organic layers were washed with brine (100 mL), dried with Na₂SO₄, filtered, and concentrated under reduced pressure. Purification by silica gel column chromatography (33% EtOAc/pet. ether) afforded desired product (6.9 g, 62% yield).

### Step 3: Synthesis of ethyl (2R,3R)-2-azido-3-cyclopropyl-3-hydroxypropanoate

A mixture of ethyl (2S,3R)-3-cyclopropyl-3-hydroxy-2-(((4-nitrophenyl)sulfonyl)oxy)propanoate (6.90 g, 19.2 mmol) and NaN₃ (6.24 g, 96.0 mmol) in DMF (70.0 mL) was heated to 50° C. The reaction mixture was stirred for 5 h and then extracted with EtOAc (3×200 mL). The combined organic layers were washed with brine (100 mL), dried with Na₂SO₄, filtered, and concentrated under reduced pressure. Purification by silica gel column chromatography (20% EtOAc/pet. ether) afforded desired product (2.8 g, 73% yield).

### Step 4: Synthesis of ethyl (2R,3S)-3-cyclopropylaziridine-2-carboxylate

A mixture of triphenylphosphine (1.84 g, 7.02 mmol) in DMF (5 mL) was stirred at 0° C. After 5 min ethyl (2R,3R)-2-azido-3-cyclopropyl-3-hydroxypropanoate (1.40 g, 7.03 mmol) was added and the reaction was warmed to room temperature. The reaction mixture was heated to 80° C. and stirred for 1 h. The mixture was then cooled to room temperature and extracted with EtOAc (3×50 mL). The combined organic layers were washed with brine (50 mL), dried with Na₂SO₄, filtered, and concentrated under reduced pressure. Purification by silica gel column chromatography (20% EtOAc/pet. ether) afforded the desired product (230 mg, 46% yield). LCMS (ESI) m/z: [M+H] calcd for C₈H₁₃NO₂: 156.10; found 156.2.

### Step 5: Synthesis of lithium (2R,3S)-3-cyclopropylaziridine-2-carboxylate

To a mixture of ethyl (2R,3S)-3-cyclopropylaziridine-2-carboxylate (230 mg, 1.5 mmol) in MeOH (3.0 mL) was added LiOH·H₂O (125 mg, 3.0 mmol). The reaction was stirred for 3 h and then filtered. The filtrate was concentrated under reduced pressure which afforded the desired product (150 mg, crude). LCMS (ESI) m/z: [M+H] calcd for C₆H₉NO₂: 128.07; found 128.2.

### Intermediate A-58. Synthesis of (2S,3R)-3-cyclopropylaziridine-2-carboxylic acid

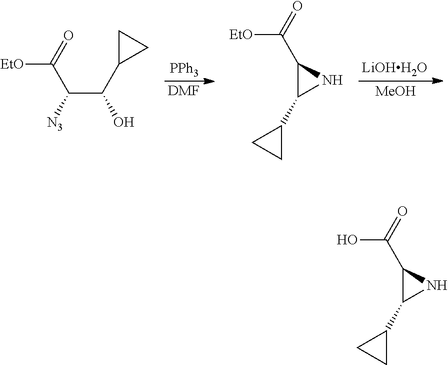

### Step 1: Synthesis of ethyl (2S,3R)-3-cyclopropylaziridine-2-carboxylate

A mixture of PPh₃ (1.4 g, 5.4 mmol) in DMF (15.0 mL) was stirred at 0° C. After 30 min, ethyl (2S,3S)-2-azido-3-cyclopropyl-3-hydroxypropanoate (980 mg, 4.92 mmol) was added. The reaction mixture was heated to 80° C. After 2 h the reaction was quenched by the addition of H₂O (20 mL) and was extracted with EtOAc (3×30 mL). Purification by silica gel column chromatography (17% EtOAc/pet. ether) afforded desired product (500 mg, 65% yield).

### Step 2: Synthesis of lithium (2S,3R)-3-cyclopropylaziridine-2-carboxylate

To a solution of ethyl (2S,3R)-3-cyclopropylaziridine-2-carboxylate (450 mg, 2.9 mmol) in THF (6.0 mL) and H₂O (2.0 mL) was added LiOH (90 mg, 3.8 mmol). The reaction was stirred for 2 h and then filtered. The filtrate was concentrated under reduced pressure which afforded the desired product (300 mg, crude).

### Intermediate A-59. Synthesis of (R)-3-methyl-2-(((R)—N-methyl-1-tritylaziridine-2-carboxamido)methyl)butanoic acid

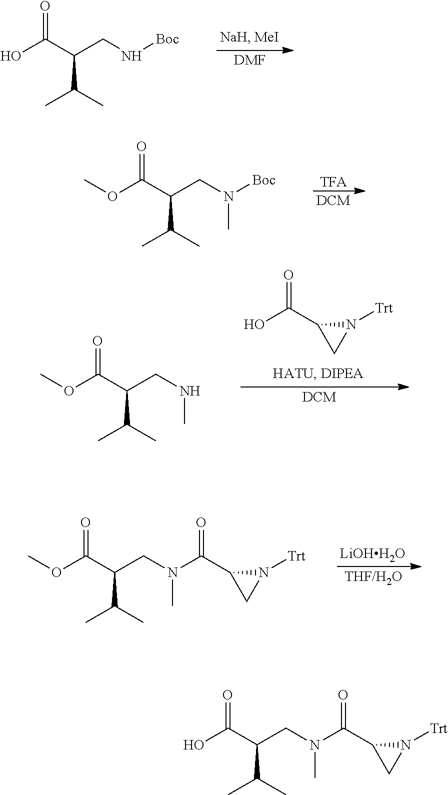

### Step 1: Synthesis of methyl (R)-2-(((tert-butoxycarbonyl)(methyl)amino)methyl)-3-methylbutanoate

To a solution of (R)-2-(((tert-butoxycarbonyl)amino)methyl)-3-methylbutanoic acid (500 mg, 2.16 mmol) in DMF (10.0 mL) at 0° C. was added NaH (130 mg, 5.40 mmol). After 30 min, Mel (540 μL, 8.65 mmol) was added and the reaction was warmed to room temperature. After 2 h the reaction was cooled to 0° C. and quenched by the addition of sat. aq. NH₄Cl (10 mL). The resulting mixture was extracted with EtOAc (3×20 mL) and the combined organic layers were washed with brine (40 mL), dried with Na₂SO₄, and concentrated under reduced pressure. Purification by prep-TLC (9% EtOAc/pet. ether) afforded the desired product (500 mg, 89.2% yield). LCMS (ESI) m/z: [M+H] calcd for C₁₃H₂₅NO₄: 260.19; found 260.2.

### Step 2: Synthesis of methyl (R)-3-methyl-2-((methylamino)methyl)butanoate

To a solution of methyl (R)-2-(((tert-butoxycarbonyl)(methyl)amino)methyl)-3-methylbutanoate (500 mg, 1.93 mmol) in DCM (5.0 mL) at 0° C. was added TFA (2.50 mL) dropwise. The resulting mixture was warmed to room temperature. After 2 h the reaction mixture was concentrated under reduced pressure to afford desired product (600 mg, crude) as a yellow solid.

### Step 3: Synthesis of methyl (R)-3-methyl-2-(((R)—N-methyl-1-tritylaziridine-2-carboxamido)methyl)butanoate

To a solution of methyl (R)-3-methyl-2-((methylamino)methyl)butanoate (550 mg, 3.45 mmol) and (R)-1-tritylaziridine-2-carboxylic acid (1.25 g, 3.80 mmol) in DCM (5.0 mL) at 0° C. was added DIPEA (1.81 mL, 10.4 mmol) followed by HATU (1.58 g, 4.15 mmol). The resulting mixture was warmed to room temperature. After 2 h the reaction was quenched with H₂O (30 mL) and extracted with EtOAc (3×30 mL). The combined organic layers were washed with brine (50 mL), dried with Na₂SO₄, and concentrated under reduced pressure. Purification by silica gel column chromatography (9% EtOAc/pet. ether) afforded the desired product (300 mg, 19% yield) as a yellow oil. LCMS (ESI) m/z: [M+H] calcd for C₃₀H₃₄N₂O₃: 471.27; found 471.3.

### Step 4: Synthesis of (R)-3-methyl-2-(((R)—N-methyl-1-tritylaziridine-2-carboxamido)methyl)butanoic acid

To a solution of methyl (R)-3-methyl-2-(((R)—N-methyl-1-tritylaziridine-2-carboxamido)methyl)butanoate (200 mg, 0.425 mmol) in THF (2.0 mL) at 0° C. was added LiOH·H₂O (89 mg, 2.13 mmol) in H₂O (2.0 mL) dropwise. The resulting mixture was warmed to room temperature. After 5 h the mixture was neutralized to pH 7 with 1M HCl. The reaction was extracted with EtOAc (3×20 mL). The combined organic layers were washed with brine (30 mL), dried with Na₂SO₄, and concentrated under reduced pressure to afford product (200 mg, crude) as an off-white solid. The crude product was used in the next step directly without further purification. LCMS (ESI) m/z: [M+H] calcd for C₂₉H₃₂N₂O₃: 457.25; found 457.2.

### Intermediate A-60. Synthesis of sodium (R)-1-methyl-5-(1-tritylaziridine-2-carboxamido)-1H-imidazole-2-carboxylate

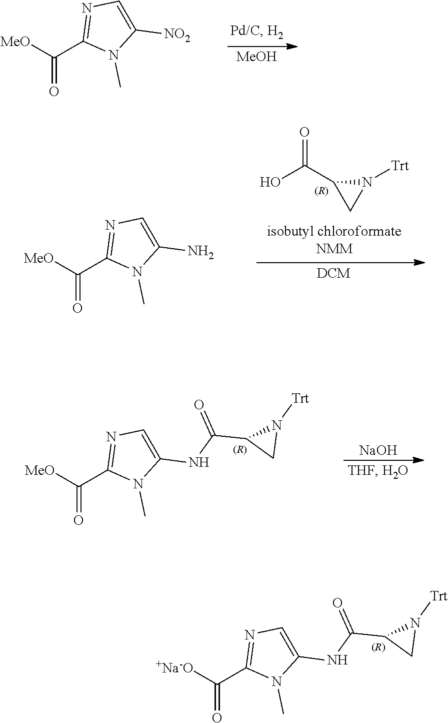

### Step 1: Synthesis of methyl 5-amino-1-methyl-1H-imidazole-2-carboxylate

A mixture of methyl 1-methyl-5-nitro-1H-imidazole-2-carboxylate (1.0 g, 5.401 mmol) and Pd/C (500.0 mg) in MeOH (15 mL) at room temperature was stirred under an atmosphere of hydrogen (1 atm) for 3 h. The mixture was filtered and the filter cake was washed with MeOH (3×20 mL). The filtrate was concentrated under reduced pressure to afford the desired product (1.0 g, crude). LCMS (ESI) m/z: [M+Na] calcd for C₆H₉N₃O₂: 156.08; found 156.1.

### Step 2: Synthesis of methyl (R)-1-methyl-5-(1-tritylaziridine-2-carboxamido)-1-imidazole-2-carboxylate

A solution of (R)-1-tritylaziridine-2-carboxylic acid (2.55 g, 7.741 mmol) in DCM (12.0 mL) at 0° C. was added in portions over 30 min to a solution of isobutyl chloroformate (845.1 mg, 6.187 mmol) and N-methylmorpholine (1.04 g, 10.282 mmol) in DCM. To the mixture was then added methyl 5-amino-1-methyl-1H-imidazole-2-carboxylate (800.0 mg, 5.156 mmol) at 0° C. The resulting mixture was warmed to room temperature and stirred overnight. The mixture was diluted with DCM (300 mL) and washed with H₂O (3×100 mL), washed with brine (2×150 mL), dried over Na₂SO₄, filtered, and concentrated under reduced pressure. The residue was purified by silica gel chromatography (25% EtOAc/hexanes) to afford the final product (1.2 g, 49.9% yield). LCMS (ESI) m/z: [M+H] calcd for C₂₈H₂₆N₄O₃: 467.21; found 467.2.

### Step 3: Synthesis of sodium (R)-1-methyl-5-(1-tritylaziridine-2-carboxamido)-1H-imidazole-2-carboxylate

To a solution of methyl (R)-1-methyl-5-(1-tritylaziridine-2-carboxamido)-1-imidazole-2-carboxylate (300.0 mg, 0.643 mmol) in THF (3 mL) at room temperature was added NaOH·H₂O (38.6 mg, 0.965 mmol). The resulting solution was warmed to room temperature and stirred for 2 h. The solution was concentrated under reduced pressure to afford the desired product (400 mg, crude). LCMS (ESI) m/z: [M+H] calcd for C₂₇H₂₄N₄O₃: 453.19; found 453.2.

### Intermediate A-61. Synthesis of (S)-1-methyl-5-(1-tritylaziridine-2-carboxamido)-1H imidazole-2-carboxylic acid

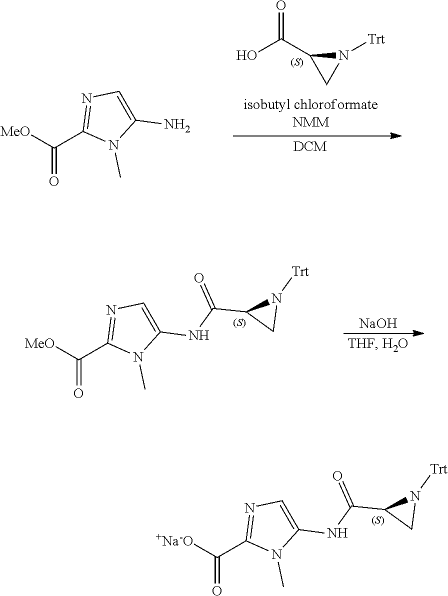

### Step 1: Synthesis of methyl (S)-1-methyl-5-(1-tritylaziridine-2-carboxamido)-1H-imidazole-2-carboxylate

To a solution of (S)-1-tritylaziridine-2-carboxylic acid (1.18 g, 3.577 mmol) in DCM (15 mL) at 0° C. was added isobutyl chloroformate (423.4 mg, 3.100 mmol) and N-methylmorpholine (0.39 mL) 3.862 mmol). The resulting mixture was stirred for 1 h at 0° C. and then methyl 5-amino-1-methyl-1H-imidazole-2-carboxylate (370.0 mg, 2.385 mmol) was added and the resulting mixture was warmed to at room temperature and stirred overnight. The reaction mixture was quenched with sat. aq. NaHCO₃ at 0° C. before being extracted with DCM (2×100 mL). The combined organic layers were washed with brine (150 mL), dried over Na₂SO₄, filtered, and concentrated under reduced pressure. The resulting residue was purified by silica gel chromatography (100% EtOAc) to afford the final product (380 mg, 34.2% yield) as a yellow solid. LCMS (ESI) m/z: [M+Na] calcd for C₂₈H₂₆N₄O₃: 467.21; found 467.3.

### Step 2: Synthesis of (S)-1-methyl-5-(1-tritylaziridine-2-carboxamido)-1H-imidazole-2-carboxylic

To a solution of NaOH (146.6 mg, 3.665 mmol) in H₂O (3.6 mL) at 0° C. was added a solution of methyl (S)-1-methyl-5-(1-tritylaziridine-2-carboxamido)-1H-imidazole-2-carboxylate (380.0 mg, 0.815 mmol) in MeOH (5 mL). The resulting solution was warmed to room temperature and stirred for 6 h. The mixture was acidified to pH 6 with aq. 1 M HCl before being extracted with DCM (2×100 mL). The combined organic layers were washed with brine (150 mL), dried over Na₂SO₄, filtered, and concentrated under reduced pressure to afford the desired product (350 mg, crude). LCMS (ESI) m/z: [M+H] calcd for C₂₇H₂₄N₄O₃: 451.18; found 451.1.

### Intermediate A-62. Synthesis of 4-((2S)-1-(tert-butylsulfinyl)aziridin-2-yl)benzoic acid

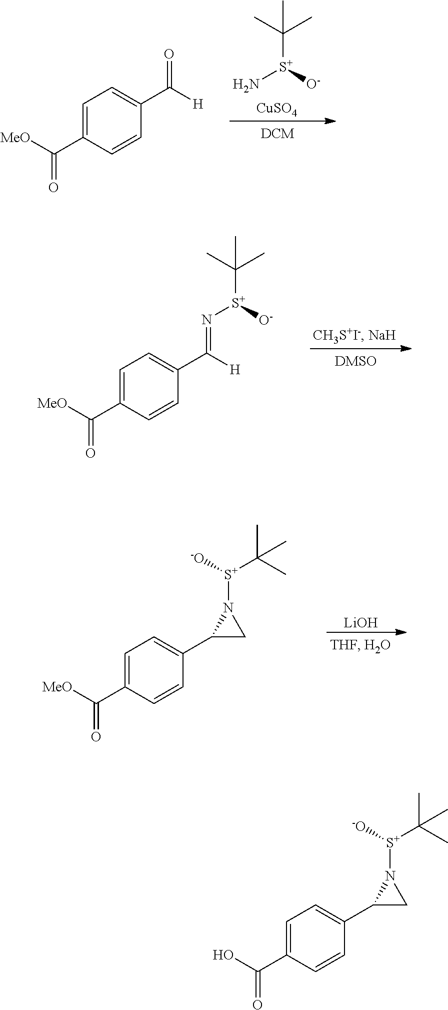

### Step 1: Synthesis of methyl (E)-4-(((tert-butylsulfinyl)imino)methyl)benzoate

To a solution of methyl 4-formylbenzoate (100.0 mg, 0.609 mmol) and 2-methylpropane-2-sulfinamide (76.0 mg, 0.627 mmol) in DCM (2.0 mL) was added CuSO₄ (291.7 mg, 1.827 mmol). The resulting solution was stirred overnight at room temperature and was then filtered. The filter cake was washed with EtOAc (3×200 mL) and the filtrate was concentrated under reduced pressure. The resulting residue was purified by silica gel column chromatography (67% EtOAc/pet. ether) to afford the desired product (2 g, 61.4% yield). LCMS (ESI) m/z: [M+H] calcd for C₁₃H₁₇NO₃S: 268.10; found 268.0.

### Step 2: Synthesis of methyl 4-((2S)-1-(tert-butylsulfinyl)aziridin-2-yl)benzoate

Methyl (E)-4-(((tert-butylsulfinyl)imino)methyl)benzoate (500.0 mg, 1.863 mmol), Me₃S⁺I⁻ (1.14 g, 5.590 mmol), and 60% NaH (134.15 mg, 5.590 mmol) were dissolved in DMSO (10.0 mL) at room temperature. The resulting mixture was stirred for 2 h and then the reaction was quenched by the addition of sat. aq. NH₄Cl (10 mL). The resulting mixture was extracted with EtOAc (3×50 mL) and the combined organic layers were washed with brine (2×100 mL), dried over Na₂SO₄, filtered, and concentrated under reduced pressure. The resulting residue was purified by silica gel chromatography (10% EtOAc/pet. ether) to afford the desired product (300 mg, 57.0% yield). LCMS (ESI) m/z: [M+H] calcd for C₁₄H₁₉NO₃S: 282.12; found 282.1.

### Step 3: Synthesis of 4-((2S)-1-(tert-butylsulfinyl)aziridin-2-yl)benzoic acid

To a solution of methyl 4-((2S)-1-(tert-butylsulfinyl)aziridin-2-yl)benzoate (400.0 mg, 1.422 mmol) in THF (5.0 mL) and H₂O (1.0 mL) was added LiOH (103.0 mg, 4.301 mmol). The resulting mixture was stirred overnight at room temperature and was then acidified to pH ˜3 with 1 M HCl. The mixture was extracted with EtOAc (3×200 mL), dried over Na₂SO₄, filtered, and concentrated under reduced pressure. The resulting residue was purified by prep-TLC (10% MeOH/DCM) to afford the desired product (130 mg, 91.2% yield). LCMS (ESI) m/z: [M−H] calcd for C₁₃H₁₇NO₃S: 266.09; found 266.0.

### Intermediate A-63. Synthesis of (S)-3-methyl-2-((R)-2-oxo-3-((R)-1-tritylaziridine-2-carboxamido)pyrrolidin-1-yl)butanoic acid

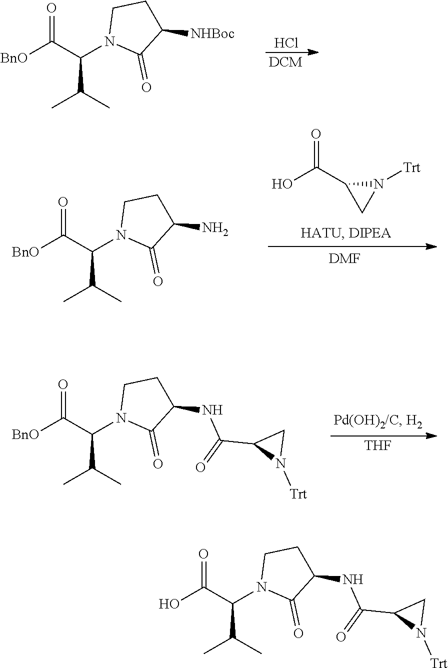

### Step 1: Synthesis of benzyl (S)-2-((R)-3-amino-2-oxopyrrolidin-1-yl)-3-methylbutanoate

To a solution of benzyl (S)-2-((R)-3-((tert-butoxycarbonyl)amino)-2-oxopyrrolidin-1-yl)-3-methylbutanoate (1.0 g, 2.561 mmol) in DCM (10.0 mL) was added 4M HCl in 1,4-dioxane (5.0 mL) at 0° C. The resulting mixture was stirred for 2 h at room temperature under an argon atmosphere. The resulting mixture was concentrated under reduced pressure to afford the desired crude product (890 mg, crude) as a yellow solid. LCMS (ESI) m/z: [M+H] calcd for C₁₆H₂₂N₂O₃: 291.17; found 291.1.

### Step 2: Synthesis of benzyl (S)-3-methyl-2-((R)-2-oxo-3-((R)-1-tritylaziridine-2-carboxamido)pyrrolidin-1-yl)butanoate

To a solution of benzyl (S)-2-((R)-3-amino-2-oxopyrrolidin-1-yl)-3-methylbutanoate (450.0 mg, 1.550 mmol) and (R)-1-tritylaziridine-2-carboxylic acid (765.8 mg, 2.325 mmol) in DMF were added HATU (1.179 g, 3.100 mmol) and DIPEA (1.35 mL, 7.75 mmol) dropwise at 0° C. The resulting mixture was stirred for 2 h at room temperature and was then extracted with EtOAc (2×100 mL). The combined organic layers were washed with H₂O, brine (20 mL), dried over Na₂SO₄, filtered, and concentrated under reduced pressure. The resulting residue was purified by silica gel chromatography (50% EtOAc/pet. ether) to afford the desired product (470 mg, 50.4% yield). LCMS (ESI) m/z: [M+H] calcd for C₃₈H₃₉N₃O₄: 602.31; found 602.3.

### Step 3: Synthesis of (S)-3-methyl-2-((R)-2-oxo-3-((R)-1-tritylaziridine-2-carboxamido)pyrrolidin-1-yl)butanoic acid

A suspension of benzyl (S)-3-methyl-2-((R)-2-oxo-3-((R)-1-tritylaziridine-2-carboxamido)pyrrolidin-1-yl)butanoate (430.0 mg, 0.715 mmol) and Pd(OH)₂/C (230.0 mg, 1.638 mmol) were in THF (5 mL) was stirred for 3 h under and atmosphere of hydrogen (1 atm). The resulting mixture was filtered and the filter cake was washed with MeOH (2×50 mL). The filtrate was concentrated under reduced pressure to afford the crude final product (16 mg, crude) as a white solid. LCMS (ESI) m/z: [M+H] calcd for C₃₁H₃₃N₃O₄: 510.24; found 510.1.

### Intermediate A-64. Synthesis of potassium (S)-1-isopropylaziridine-2-carboxylate

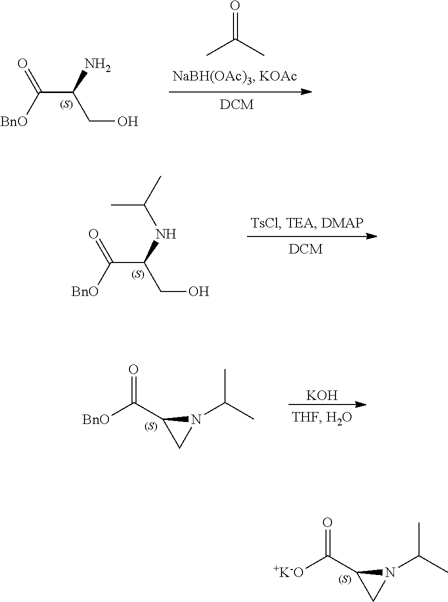

### Step 1: Synthesis benzyl isopropyl-L-serinate

To a solution of benzyl L-serinate (3.65 g, 18.69 mmol), KOAc (1.83 g, 18.69 mmol), and acetone (2.5 mL, 33.66 mmol) in DCM (60.0 mL) was added NaBH(AcO)₃ (4.76 g, 22.436 mmol) in portions at 0° C. The resulting mixture was stirred overnight at room temperature. The reaction was quenched by the addition of sat. aq. NaHCO₃ (50 mL) at room temperature. The resulting mixture was extracted with DCM (3×80 mL). The combined organic layers were washed with brine (50 mL), dried over Na₂SO₄, filtered, and concentrated under reduced pressure. The residue was purified by silica gel chromatography (67% EtOAc/hexanes) to afford the desired product (2.7 g, 60.9% yield) as an off-white solid. LCMS (ESI) m/z: [M+H] calcd for C₁₃H₁₉NO₃: 238.14; found 238.2.

### Step 2: Synthesis of benzyl (S)-1-isopropylaziridine-2-carboxylate

To a solution of benzyl isopropyl-L-serinate (2.70 g, 11.378 mmol), Et₃N (4.75 mL, 34.134 mmol) and DMAP (2.57 mg, 0.021 mmol) in DCM (50.0 mL) was added a solution of TsCl (2.60 g, 13.65 mmol) in DCM dropwise at 0° C. The resulting mixture was stirred overnight at room temperature and was then stirred for 4 h at 40° C. The reaction mixture was diluted with H₂O (80 mL) and was then extracted with DCM (2×50 mL). The combined organic layers were washed with brine (30 mL), dried over Na₂SO₄, filtered, and concentrated under reduced pressure. The residue was purified by silica gel chromatography (20% EtOAc/hexanes) to afford the desired product (2.3 g, 93.2% yield). LCMS (ESI) m/z: [M+H] calcd for C₁₃H₁₇NO₂: 220.13; found 220.1.

### Step 3: Synthesis of potassium (S)-1-isopropylaziridine-2-carboxylate

To a solution of benzyl (S)-1-isopropylaziridine-2-carboxylate (800.0 mg, 3.65 mmol) and H₂O (6.0 mL) and THF (8.0 mL) was added a solution of KOH (245.62 mg, 4.378 mmol) in H₂O (2.0 mL) dropwise at 0° C. The resulting mixture was stirred for 2 h at room temperature. The mixture was diluted with H₂O (10 mL) and the aqueous layer was washed with MTBE (3×8 mL). The aqueous layer was dried by lyophilization to afford the desired product (400 mg, crude). LCMS (ESI) m/z: [M+H] calcd for C₆H₁₁NO₂: 130.09; found 130.0.

### Intermediate A-65. Synthesis of potassium (R)-1-isopropylaziridine-2-carboxylate

### Step 1: Synthesis benzyl isopropyl-D-serinate

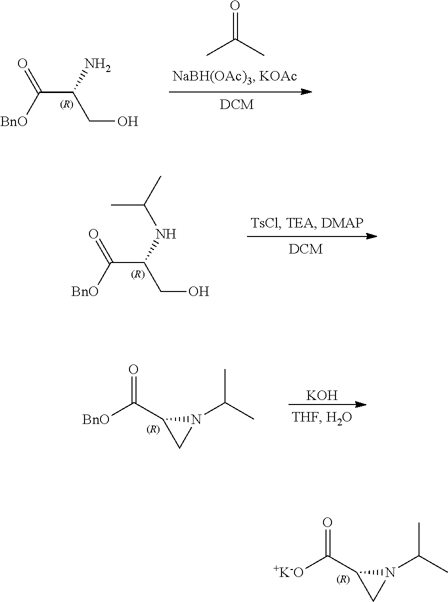

To a solution of benzyl D-serinate (2.10 g, 10.757 mmol), KOAc (1.06 g, 10.757 mmol), and acetone (1.2 mL, 16.136 mmol) in DCM (40.0 mL) was added a solution of NaBH(AcO)₃ (2.96 g, 13.984 mmol) in portions at 0° C. The resulting mixture was stirred overnight at room temperature. The reaction was quenched by the addition of sat. aq. NaHCO₃ (50 mL) and the mixture was extracted with DCM (3×50 mL). The combined organic layers were washed with brine (50 mL), dried over Na₂SO₄, filtered, and concentrated under reduced pressure. The residue was purified by silica gel chromatography (67% EtOAc/hexanes) to afford the desired product (1.7 g, 66.6% yield). LCMS (ESI) m/z: [M+H] calcd for C₁₃H₁₉NO₃: 238.14; found 238.0.

### Step 2: Synthesis of benzyl (R)-1-isopropylaziridine-2-carboxylate

To a solution of benzyl isopropyl-D-serinate (1.75 g, 7.375 mmol), Et₃N (2.58 mL, 18.437 mmol) and DMAP (90.09 mg, 0.737 mmol) in DCM (30.0 mL) was added a solution of TsCl (1.69 g, 8.850 mmol) in DCM dropwise at 0° C. The resulting mixture was stirred overnight at room temperature before being stirred for 4 h at 40° C. The mixture was diluted with H₂O (80 mL) and then extracted with DCM (3×50 mL). The combined organic layers were washed with brine (50 mL), dried over Na₂SO₄, filtered, and concentrated under reduced pressure. The residue was purified by silica gel chromatography (20% EtOAc/hexanes) to afford the desired product (1.4 g, 86.6% yield). LCMS (ESI) m/z: [M+H] calcd for C₁₃H₁₇NO₂: 220.13; found 219.9.

### Step 3: Synthesis of potassium (R)-1-isopropylaziridine-2-carboxylate

To a solution of benzyl (R)-1-isopropylaziridine-2-carboxylate (600.0 mg, 2.736 mmol) in H₂O (3.0 mL) and THF (5.0 mL) was added a solution of KOH (184.22 mg, 3.283 mmol) in H₂O (2.0 mL) dropwise at 0° C. The resulting mixture was stirred for 2 h at room temperature. The mixture was then diluted with H₂O (10 mL) and the aqueous layer was washed with MTBE (3×8 mL). The aqueous layer was then dried by lyophilization to afford the desired product (260 mg, crude). LCMS (ESI) m/z: [M+H] calcd for C₆H₁₁NO₂: 130.09; found 130.1.

### Intermediate A-66. Synthesis of N-methyl-N-(3-oxo-4-((R)-1-tritylaziridine-2-carbonyl)piperazine-1-carbonyl)-L-valine

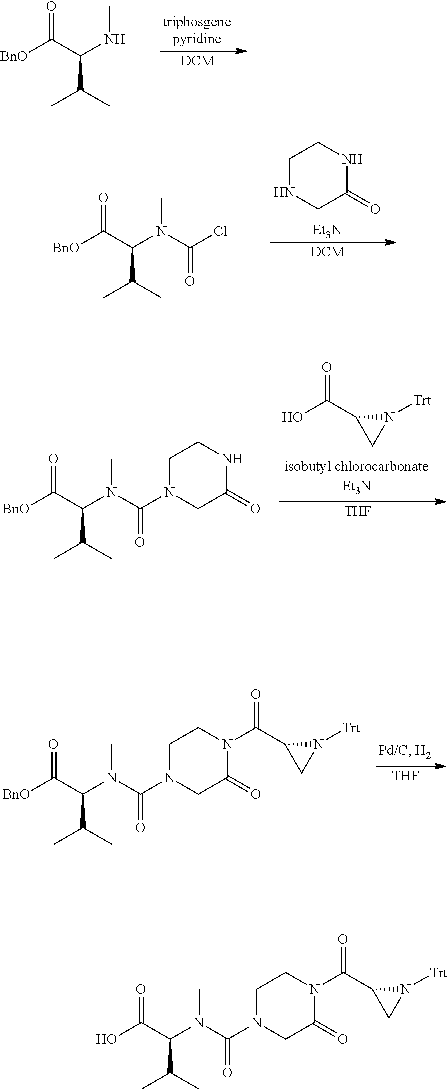

### Step 1: Synthesis of benzyl N-(chlorocarbonyl)-N-methyl-L-valinate

To a solution of benzyl methyl-L-valinate (2.0 g, 9.038 mmol) in DCM (20.0 mL) was added a solution of triphosgene (800 mg, 2.711 mmol) and pyridine (2.14 g, 27.113 mmol) in DCM (20.0 mL) dropwise at 0° C. The resulting mixture was stirred for 2 h at room temperature before being diluted with EtOAc. The solution was stirred for 30 min at room temperature and was then filtered. The filtrate was concentrated under reduced pressure to afford the crude product which was used in the next step directly without further purification. LCMS (ESI) m/z: [M+H] calcd for C₁₄H₁₈ClNO₃: 284.11; found 283.1.

### Step 2: Synthesis of benzyl N-methyl-N-(3-oxopiperazine-1-carbonyl)-L-valinate

To a solution of piperazin-2-one (100.0 mg, 0.999 mmol) and Et₃N (0.487 mL, 3.496 mmol) in DCM (5.0 mL) was added a solution of benzyl N-(chlorocarbonyl)-N-methyl-L-valinate (311.75 mg, 1.099 mmol) in DCM (5 mL) dropwise at 0° C. The resulting mixture was stirred for 4 h at room temperature. The mixture was then diluted with H₂O (5 mL), the aqueous layer was extracted with DCM (3×5 mL), and the combined organic layers were concentrated under reduced pressure. The residue was purified by prep-TLC (100% EtOAc) to afford the desired product (200 mg, 57.6% yield). LCMS (ESI) m/z: [M+H] calcd for C₁₈H₂₅N₃O₄: 348.19; found 348.1.

### Step 3: Synthesis of benzyl N-methyl-N-(3-oxo-4-((R)-1-tritylaziridine-2-carbonyl)piperazine-1-carbonyl)-L-valinate

To a solution of (R)-1-tritylaziridine-2-carboxylic acid (711.11 mg, 2.159 mmol) in THF was added Et₃N (0.40 mL, 2.878 mmol) and isobutyl chlorocarbonate (255.53 mg, 1.871 mmol) dropwise at 0° C. under a nitrogen atmosphere. The resulting mixture was stirred for 1 h at room temperature and then benzyl N-methyl-N-(3-oxopiperazine-1-carbonyl)-L-valinate (500.0 mg, 1.439 mmol) was added at room temperature. The resulting mixture was warmed to 70° C. and stirred overnight. The reaction was then cooled to room temperature and concentrated under reduced pressure. The residue was purified by prep-TLC (EtOAc/50% pet. ether) to afford the desired product (200 mg, 21.1% yield). LCMS (ESI) m/z: [M+H] calcd for C₄₀H₄₂N₄O₅: 659.32; found 677.4.

### Step 4: Synthesis of N-methyl-N-(3-oxo-4-((R)-1-tritylaziridine-2-carbonyl)piperazine-1-carbonyl)-L-valine

A suspension of benzyl N-methyl-N-(3-oxo-4-((R)-1-tritylaziridine-2-carbonyl)piperazine-1-carbonyl)-L-valinate (140.0 mg, 0.212 mmol) and Pd/C (50.0 mg) in THF (3 mL) was stirred for 2 h under a hydrogen atmosphere (1 atm). The mixture was then filtered and the filter cake was washed with MeOH (3×15 mL). The filtrate was concentrated under reduced pressure to afford the desired product (100 mg, crude). LCMS (ESI) m/z: [M−H] calcd for C₃₃H₃₆N₄O₅: 567.26; found 567.1.

### Intermediate A-67. Synthesis of (S)-1-(2-((tert-butyldiphenylsilyl)oxy)ethyl)aziridine-2-carboxylic acid

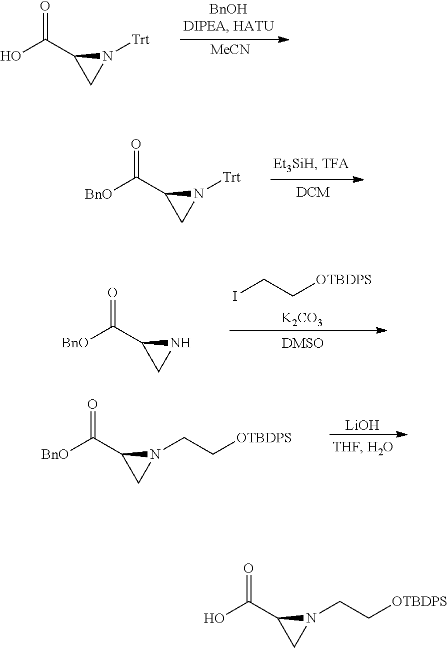

### Step 1: Synthesis of benzyl (S)-1-tritylaziridine-2-carboxylate

To a solution of (S)-1-tritylaziridine-2-carboxylic acid (500.0 mg, 1.518 mmol), benzyl alcohol (246.2 mg, 2.277 mmol) and DIPEA (0.793 mL, 4.554 mmol) in MeCN (10.0 mL) was added HATU (1.73 mg, 4.554 mmol). The resulting solution was stirred for 3 h at room temperature and was then concentrated under reduced pressure. The crude residue was purified by prep-TLC (50% EtOAc/pet. ether) to afford the desired product (300 mg, 47.1% yield) as an off-white slid. LCMS (ESI) m/z: [M+Na] calcd for C₂₉H₂₅NO₂: 442.18; found 442.3.

### Step 2: Synthesis of benzyl (S)-aziridine-2-carboxylate

To a solution of benzyl (S)-1-tritylaziridine-2-carboxylate (300.0 mg, 0.715 mmol) in DCM (5.0 mL) at 0° C. was added TFA (326.2 mg, 2.860 mmol) and Et₃SiH (332.6 mg, 2.860 mmol). The resulting mixture was stirred at 0° C. for 3 h and was then concentrated under reduced pressure. The residue was purified by prep-TLC (10% MeOH/DCM) to afford the desired product (130 mg, 82.1% yield). LCMS (ESI) m/z: [M+H] calcd for C₁₀H₁₁NO₂: 178.09; found 178.2.

### Step 3: Synthesis of benzyl (S)-1-(2-((tert-butyldiphenylsilyl)oxy)ethyl)aziridine-2-carboxylate

To a solution of benzyl (S)-aziridine-2-carboxylate (400.0 mg, 2.257 mmol) and tert-butyl(2-iodoethoxy)diphenylsilane (1.85 g, 4.52 mmol) in DMSO (10.0 mL) was added K₂CO₃ (935.9 mg, 6.772 mmol) at room temperature. The mixture was stirred at 60° C. for 5 h. The mixture was diluted with H₂O (30.0 mL) and was extracted with EtOAc (2×30 mL). The combined organic layers were washed with brine (2×50 mL), dried over Na₂SO₄, filtered, and concentrated under reduced pressure. The resulting residue was purified by prep-TLC (20% EtOAc/pet. ether) to afford the desired product (200 mg, 15.4% yield). LCMS (ESI) m/z: [M+H] calcd for C₂₈H₃₃NO₃Si: 460.23; found 460.0.

### Step 4: Synthesis of lithium (S)-1-(2-((tert-butyldiphenylsilyl)oxy)ethyl)aziridine-2-carboxylate

To a solution of benzyl (S)-1-(2-((tert-butyldiphenylsilyl)oxy)ethyl)aziridine-2-carboxylate (200.0 mg, 0.435 mmol) in MeOH (2.0 mL) was added LiOH·H₂O (36.5 mg, 0.870 mmol). The resulting mixture was stirred overnight and was then concentrated under reduced pressure to afford the desired product (200 mg, crude). LCMS (ESI) m/z: [M+H] calcd for C₂₁H₂₇NO₃Si: 370.18; found 370.1.

### Intermediate A-68. Synthesis of (R)-1-(2-((ter-butyldiphenylsilyl)oxy)ethyl)aziridine-2-carboxylic acid

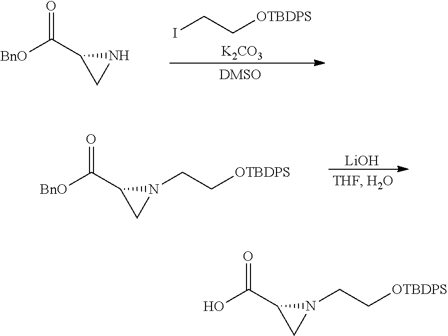

### Step 1: Synthesis of methyl benzyl (R)-1-(2-((tert-butyldiphenylsilyl)oxy)ethyl)aziridine-2-carboxylate

To a solution of benzyl (R)-aziridine-2-carboxylate (600.0 mg, 3.386 mmol) and K₂CO₃ (1.87 g, 13.544 mmol) in DMSO (8.0 mL) was added tert-butyl(2-iodoethoxy)diphenylsilane (1.39 g, 3.386 mmol) in portions at room temperature. The resulting mixture was stirred at 80° C. for 16 h. The reaction mixture was then cooled to room temperature and concentrated under reduced pressure. The residue was purified by reverse phase chromatography (60→90% MeCN/H₂O) to afford the desired product (150 mg, 9.6% yield) as a colorless solid. LCMS (ESI) m/z: [M+Na] calcd for C₂₈H₃₃NO₃Si: 482.21; found 482.3.

### Step 2: Synthesis of (R)-1-(2-((tert-butyldiphenylsilyl)oxy)ethyl)aziridine-2-carboxylic acid

To a solution of methyl benzyl (R)-1-(2-((tert-butyldiphenylsilyl)oxy)ethyl)aziridine-2-carboxylate (180.0 mg, 0.392 mmol) in H₂O (2.0 mL) and THF (3.0 mL) at 0° C. was added a solution of LiOH·H₂O (32.87 mg, 0.392 mmol) in H₂O (1.0 mL). The resulting mixture was diluted with H₂O (6.0 mL) and the aqueous layer was washed with MTBE (3×4 mL). The aqueous layer was dried by lyophilization which afforded the desired product (140 mg, crude). LCMS (ESI) m/z: [M+H] calcd for C₂₁H₂₇NO₃Si: 370.18; found 370.0.

### Intermediate A-69. Synthesis of 6-((2S)-1-(tert-butylsulfinyl)aziridin-2-yl)nicotinic acid

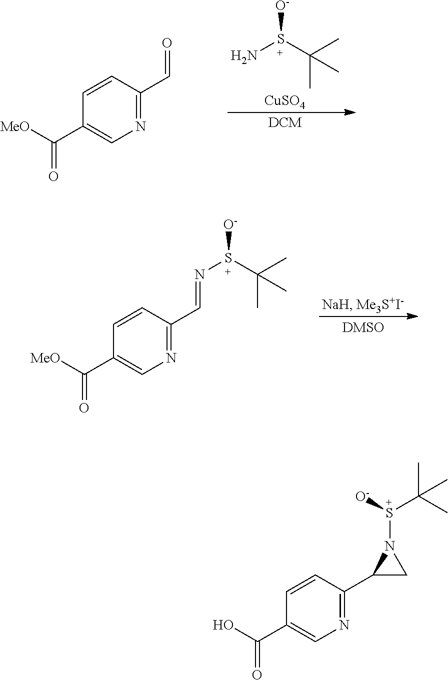

### Step 1: Synthesis of methyl (E)-6-(((tert-butylsulfinyl)imino)methyl)nicotinate

To a mixture of methyl 6-formylnicotinate (2.0 g, 12.11 mmol) and 2-methylpropane-2-sulfinamide (2.94 g, 24.26 mmol) in DCM (60 mL) was added CuSO₄ (5.80 g, 36.34 mmol). The resulting mixture was stirred at room temperature for 18 h. The reaction mixture was filtered, the filter cake was washed with DCM (3×30 mL), and the filtrate was concentrated under reduced pressure. Purification by normal phase chromatography (66% EtOAc/pet. ether) afforded the desired product (2.581 g, 80% yield). LCMS (ESI) m/z: [M+H] calcd for C₁₂H₁₆N₂O₃S: 269.10; found 269.1.

### Step 2: Synthesis of 6-((2S)-1-(tert-butylsulfinyl)aziridin-2-yl)nicotinic acid

To a suspension of NaH (60%, 179.76 mg, 7.491 mmol) in DMSO (20 mL) at 0° C. was added Me₃S⁺I⁻ (1.53 g, 7.491 mmol) and the resulting mixture was warmed to room temperature and stirred for 1 h. To the reaction mixture was added a solution of methyl (E)-6-(((tert-butylsulfinyl)imino)methyl)nicotinate (670.0 mg, 2.497 mmol) in DMSO (20 mL) in portions. The mixture was stirred at room temperature for 3 h and was then diluted with EtOAc. The mixture was acidified to pH 4 with 1 M HCl and then the aqueous layer was extracted with EtOAc. The combined organic layers were concentrated under reduced pressure. Purification by reverse phase chromatography (10→15% MeCN/H₂O) afforded the desired product (313 mg, 45% yield). LCMS (ESI) m/z: [M+H] calcd for C₁₂H₁₆N₂O₃S: 269.10; found 269.1.

### Intermediate A-70. Synthesis of N-methyl-N—(N-methyl-N—((R)-1-tritylaziridine-2-carbonyl)-D-alanyl)-L-valine

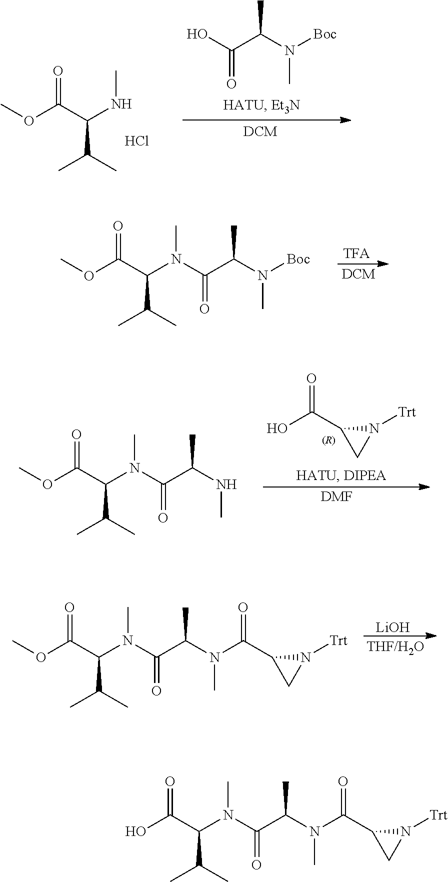

### Step 1: Synthesis of methyl N—(N-(tert-butoxycarbonyl)-N-methyl-D-alanyl)-N-methyl-L-valinate

To a solution of methyl-L-valinate hydrochloride (1.0 g, 5.51 mmol) and N-(tert-butoxycarbonyl)-N-methyl-D-alanine (1.34 g, 6.59 mmo) in DCM (20.0 mL) at 0° C. was added Et₃N (2.3 mL, 16.51 mmol) and HATU (2.72 g, 7.16 mmol). The mixture was warmed to room temperature and stirred for 4 h. The reaction mixture was then diluted with DCM (20 mL) and washed with sat. aq. NH₄Cl (2×40 mL) and brine (40 mL), dried over Na₂SO₄, filtered, and concentrated under reduced pressure. The residue was purified by silica gel column chromatography (20% EtOAc/pet. ether) to afford the desired product (1.5 g, 82.5% yield). LCMS (ESI) m/z: [M+H] calcd for C₁₆H₃₀N₂O₅: 331.23; found 331.1.

### Step 2: Synthesis of methyl N-methyl-N-(methyl-D-alanyl)-L-valinate

To a solution of methyl N—(N-(tert-butoxycarbonyl)-N-methyl-D-alanyl)-N-methyl-L-valinate (1.50 g, 4.54 mmol) in DCM (9.0 mL) at 0° C. was added TFA (4.5 mL). The resulting mixture was warmed to room temperature and stirred for 1 h. The reaction mixture was concentrated under reduced pressure to afford the desired product (1 g, crude). LCMS (ESI) m/z: [M+H] calcd for C₁₁H₂₂N₂O₃: 231.17; found 231.2.

### Step 3: Synthesis of methyl N-methyl-N—(N-methyl-N—((R)-1-tritylaziridine-2-carbonyl)-D-alanyl)-L-valinate

To a solution of methyl N-methyl-N-(methyl-D-alanyl)-L-valinate (900.0 mg, 3.91 mmol) and (R)-1-tritylaziridine-2-carboxylic acid (1.544 g, 4.689 mmol) in DMF (20.0 mL) at 0° C. was added DIPEA (3.4 mL, 19.54 mmol) and HATU (2.228 g, 5.86 mmol). The mixture was warmed to room temperature and stirred for 1 h. The reaction mixture was diluted with H₂O (50 mL) and extracted with EtOAc (3×50 mL). The combined organic layers were dried over Na₂SO₄, filtered, and concentrated under reduced pressure. The residue was purified by silica gel column chromatography (33% EtOAc/pet. ether) to afford the desired product (1.2 g, 56.7% yield). LCMS (ESI) m/z: [M+H] calcd for C₃₃H₃₉N₃O₄: 542.30; found 542.3.

### Step 4: Synthesis of N-methyl-N—(N-methyl-N—((R)-1-tritylaziridine-2-carbonyl)-D-alanyl)-L-valine

To a solution of methyl N-methyl-N—(N-methyl-N—((R)-1-tritylaziridine-2-carbonyl)-D-alanyl)-L-valinate (200.0 mg, 0.369 mmol) in THF (2.0 mL) at 0° C. was added a solution of LiOH·H₂O (77 mg, 1.84 mmol) in H₂O (1.85 mL). The resulting mixture was warmed to room temperature and stirred overnight. The mixture was adjusted to pH 9 with 1 M HCl and then adjusted to pH 7 with aq. NH₄Cl. The aqueous layer was extracted with EtOAc (3×20 mL). The combined organic layers were dried over Na₂SO₄, filtered, and concentrated under reduced pressure to afford the desired product (200 mg, crude. LCMS (ESI) m/z: [M+H] calcd for C₃₂H₃₇N₃O₄: 528.29; found 528.3.

### Intermediate A-71 and A-72. Synthesis of (2R,3S)-1-(4-methoxybenzyl)-3-(trifluoromethyl)aziridine-2-carboxylic acid and (2S,3R)-1-(4-methoxybenzyl)-3-(trifluoromethyl)aziridine-2-carboxylic acid

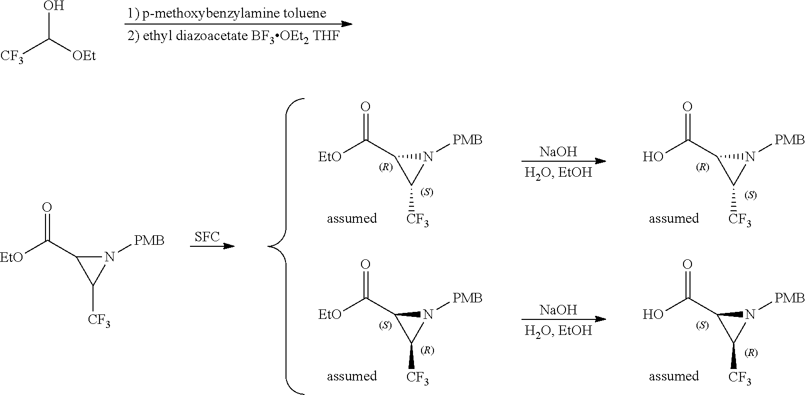

### Step 1: Synthesis of ethyl 1-(4-methoxybenzyl)-3-(trifluoromethyl)aziridine-2-carboxylate

A solution of 1-ethoxy-2,2,2-trifluoroethan-1-ol (2.17 mL, 18.37 mmol) and p-methoxybenzylamine (1.89 mL, 14.58 mmol) in toluene (46 mL) was refluxed for 16 h under Dean-Stark conditions. The reaction was concentrated under reduced pressure and the resulting residue was dissolved in THF (80 mL) and cooled to −78° C. BF₃·Et₂O (0.360 mL, 2.92 mmol) was added to the solution, followed by dropwise addition of ethyl diazoacetate (1.83 mL, 17.50 mmol). The reaction was stirred for 4 h at room temperature. The reaction mixture was quenched by addition of aq. sat. NaHCO₃ (5 mL), and the resulting solution was extracted with DCM (3×50 mL). The combined organic layers were washed with H₂O (20 mL) and brine (10 mL). The organic phase was dried over Na₂SO₄, filtered, and concentrated under reduced pressure. The residue was purified by silica gel column chromatography (1→10% EtOAc/pet. ether) afford the desired product (2 g, 45.2 yield).

### Step 2: Synthesis of ethyl (2R,3S)-1-(4-methoxybenzyl)-3-(trifluoromethyl)aziridine-2-carboxylate and ethyl (2S,3R)-1-(4-methoxybenzyl)-3-(trifluoromethyl)aziridine-2-carboxylate

Ethyl 1-(4-methoxybenzyl)-3-(trifluoromethyl)aziridine-2-carboxylate (1 g) was purified by SFC separation (column: REGIS(S,S)WHELK-O1 (250 mm*25 mm, 10 um); mobile phase: [Neu-IPA]; B %: 13%—13%, min) to afford ethyl (2R,3S)-1-(4-methoxybenzyl)-3-(trifluoromethyl)aziridine-2-carboxylate (530 mg) and ethyl (2S,3R)-1-(4-methoxybenzyl)-3-(trifluoromethyl)aziridine-2-carboxylate (470 mg).

### Step 3: Synthesis of (2R,3S)-1-(4-methoxybenzyl)-3-(trifluoromethyl)aziridine-2-carboxylic acid

To a solution of ethyl (2R,3S)-1-(4-methoxybenzyl)-3-(trifluoromethyl)aziridine-2-carboxylate (430 mg, 1.42 mmol) in EtOH (4 mL) and H₂O (6 mL) was added NaOH (113.42 mg, 2.84 mmol). The mixture was stirred at room temperature for 5 h. The mixture was acidified with aq. HCl (2M) to pH=1-2. The reaction mixture was poured into H₂O (3 mL) and the aqueous phase was extracted with EtOAc (3×3 mL). The combined organic phase was washed with brine (5 mL), dried over Na₂SO₄, filtered, and concentrated under reduced pressure to afford the desired product (350 mg, 89.1% yield). LCMS (ESI) m/z: [M+H] calcd for C₁₂H₁₁FNO₃:274.08; found 274.1.

### Step 4: Synthesis of (2S,3R)-1-(4-methoxybenzyl)-3-(trifluoromethyl)aziridine-2-carboxylic acid

To a solution of ethyl (2S,3R)-1-(4-methoxybenzyl)-3-(trifluoromethyl)aziridine-2-carboxylate (370 mg, 1.22 mmol) in H₂O (2 mL) and EtOH (4 mL) was added NaOH (97.59 mg, 2.44 mmol). The mixture was stirred at room temperature for 5 h. The mixture was brought to pH=1-2 with the addition of aq. HCl (2 M). The reaction mixture was poured into H₂O (3 mL) and the aqueous phase was extracted with EtOAc (3×3 mL). The combined organic phase was washed with brine (5 mL), dried over Na₂SO₄, filtered, and concentrated under reduced pressure to afford the desired product (300 mg, 89.0% yield). LCMS (ESI) m/z: [M+H] calcd for C₁₂H₁₁FNO₃:234.08; found 234.2.

### Intermediate A-73 and A-74. Synthesis of (2S,3S)-1-benzyl-3-(trifluoromethyl)aziridine-2-carboxylic acid and (2R,3R)-1-benzyl-3-(trifluoromethyl)aziridine-2-carboxylic acid

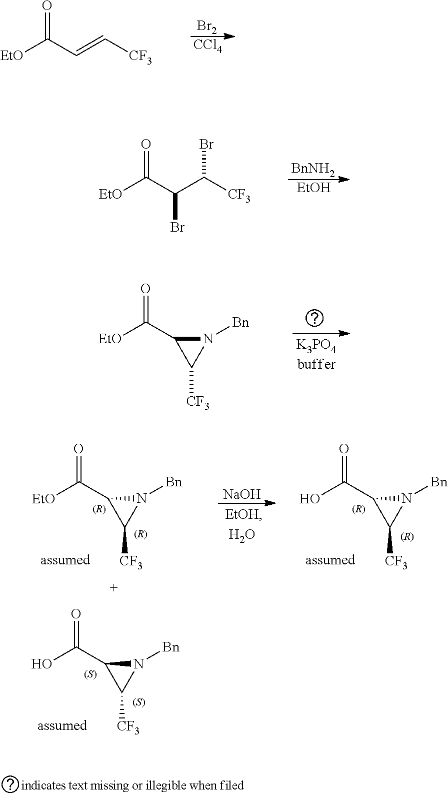

### Step 1: Synthesis of ethyl (2S,3R)-2,3-dibromo-4,4,4-trifluorobutanoate

To a solution of ethyl (E)-4,4,4-trifluorobut-2-enoate (5 g, 29.74 mmol, 4.42 mL) in CCl₄ (90 mL) was added Br₂ (1.69 mL, 32.72 mmol) and the solution was stirred at 75° C. for 5 h. The reaction mixture was concentrated under reduced pressure to give the desired product (10.72 g, crude).

### Step 2: Synthesis of ethyl (2S,3S)-1-benzyl-3-(trifluoromethyl)aziridine-2-carboxylate

To a solution of ethyl (2S,3R)-2,3-dibromo-4,4,4-trifluorobutanoate (10.72 g, 32.69 mmol) in EtOH (30 mL) was slowly added the solution of BnNH₂ (12.47 mL, 114.42 mmol) in EtOH (120 mL) at −5° C. under N₂. The mixture was warmed to room temperature and stirred for 15 h. The mixture was concentrated under reduced pressure and EtOAc (120 mL) was added to the residue. The precipitate was filtered off and the filtrate was washed with aqueous HCl (3%, 180 mL) and H₂O (100 mL), dried over Na₂SO₄, filtered and concentrated under reduced pressure. The residue was purified by silica gel chromatography (20% EtOAc/pet. ether) to afford the desired product (6.02 g, 67.4% yield).

### Step 3: Synthesis of ethyl (2R,3R)-1-benzyl-3-(trifluoromethyl)aziridine-2-carboxylate and (2S,3S)-1-benzyl-3-(trifluoromethyl)aziridine-2-carboxylic acid

Ethyl (2R,3R)-1-benzyl-3-(trifluoromethyl)aziridine-2-carboxylate and (2S,3S)-1-benzyl-3-(trifluoromethyl)aziridine-2-carboxylic acid were synthesized in Enzyme Screening Platform, based on the procedure in Tetrahedron Asymmetry 1999, 10, 2361.

### Step 5: Synthesis of (2R,3R)-1-benzyl-3-(trifluoromethyl)aziridine-2-carboxylic acid

To a solution of ethyl (2R,3R)-1-benzyl-3-(trifluoromethyl)aziridine-2-carboxylate (200 mg, 731.93 μmol) in EtOH (5 mL) was added NaOH (2 M, 548.95 μL) and the mixture was stirred at room temperature for 1 h. The reaction mixture was concentrated under reduced pressure to remove EtOH. Then to the mixture was added HCl (1 M) to adjust pH to 1, and extracted with EtOAc (3×5 mL). The combined organic layers were washed with brine (2×10 mL), dried over Na₂SO₄, filtered and concentrated under reduced pressure to afford the desired product (138 mg, 76.9% yield). LCMS (ESI) m/z: [M+H] calcd for C₁₁H₁₀F₃NO₂: 246.07; found 245.9.

### Intermediate A-75. Synthesis of 1-(oxetan-3-yl)aziridine-2-carboxylic acid

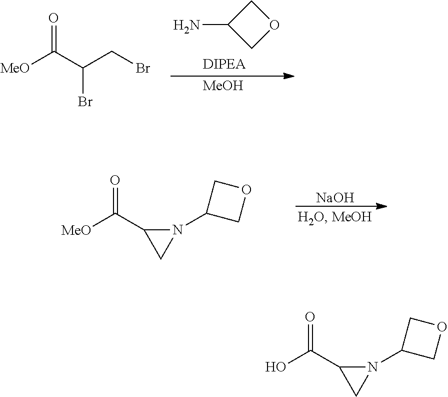 [SMILES: "MeOH" ; COC(=O)C(Br)CBr ; NC1COC1 ; COC(=O)C1CN1C1COC1 ; "NaOH" ; O=C(O)C1CN1C1COC1]

### Step 1: Synthesis of methyl 1-(oxetan-3-yl)aziridine-2-carboxylate

To a solution of methyl 2,3-dibromopropanoate (515.46 μL, 4.07 mmol) in MeOH (15 mL) was added DIPEA (3.54 mL, 20.33 mmol). After addition, the mixture was stirred for 15 min, and then oxetan-3-amine (297.25 mg, 4.07 mmol) was added dropwise. The resulting mixture was stirred at room temperature for 12 h. The reaction mixture was poured into H₂O (20 mL), the aqueous phase was extracted with DCM (2×25 mL). The combined organic phase was washed with brine (20 mL), dried with Na₂SO₄, filtered and concentrated under reduced pressure. The residue was purified by silica gel chromatography (10%→30% EtOAc/pet. ether) to afford the desired product (380 mg, 59.5% yield).

### Step 2: Synthesis of 1-(oxetan-3-yl)aziridine-2-carboxylic acid

To a solution of methyl 1-(oxetan-3-yl)aziridine-2-carboxylate (280 mg, 1.78 mmol) in EtOH (3 mL) was added NaOH (2 M, 1.34 mL) at room temperature and the resulting mixture was stirred for 3 h. The reaction mixture was adjusted to pH 8 by the addition of HCl (1 M), and lyophilized to afford the desired product (200 mg, 78.4% yield).

### Intermediate A-76. Synthesis of (2S,3S)-1-((S)-tert-butylsulfinyl)-3-cyclobutylaziridine-2-carboxylic acid

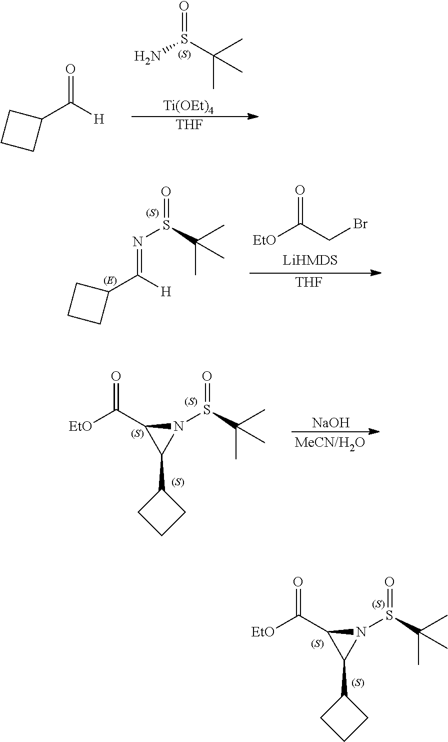

### Step 1: Synthesis of (S,E)-N-(cyclobutylmethylene)-2-methylpropane-2-sulfinamide

To a solution of cyclobutanecarbaldehyde (0.5 g, 5.94 mmol) in THF (10 mL) was added (S)-2-methylpropane-2-sulfinamide (792.48 mg, 6.54 mmol) and Ti(OEt)₄ (2.47 mL, 11.89 mmol). The mixture was stirred at 75° C. for 3 h. The reaction mixture was cooled to room temperature and quenched by addition brine (30 mL), and filtered to remove solids. The mixture was extracted with EtOAc (3×30 mL). The combined organic layers were washed with brine (2×10 mL), dried over Na₂SO₄, filtered and concentrated under reduced pressure to give a residue. The residue was purified by silica gel chromatography (2%→10% EtOAc/pet. ether) to afford the desired product (907.3 mg, 39.9% yield). LCMS (ESI) m/z: [M+H] calcd for C₉H₁₇NOS: 188.1; found 188.3.

### Step 2: Synthesis of ethyl (2S,3S)-1-((S)-tert-butylsulfinyl)-3-cyclobutylaziridine-2-carboxylate

To a solution of ethyl 2-bromoacetate (1.60 g, 9.61 mmol, 1.06 mL) in THF (9 mL) was added LiHMDS (1 M, 9.61 mL) at −78° C., after 2 min, (S,E)-N-(cyclobutylmethylene)-2-methylpropane-2-sulfinamide (0.9 g, 4.81 mmol) was added. The mixture was stirred at −78° C. for 2 h. The reaction mixture was quenched by addition H₂O (25 mL) at −78° C. and warmed to room temperature, then the mixture extracted with EtOAc (3×20 mL). The combined organic layers were washed with brine (2×5 mL), dried over Na₂SO₄, filtered and concentrated under reduced pressure to give a residue, which was purified by silica gel chromatography (10%→20% EtOAc/pet. ether) to afford the desired product (426 mg, crude). LCMS (ESI) m/z: [M+H] calcd for C₁₃H₂₃NO₃S: 274.14; found 274.3.

### Step 3: Synthesis of (2S,3S)-1-((S)-tert-butylsulfinyl)-3-cyclobutylaziridine-2-carboxylic acid

To a solution of (2S,3S)-1-((S)-tert-butylsulfinyl)-3-cyclobutylaziridine-2-carboxylate (100 mg, 365.78 μmol) in MeCN (0.5 mL) and H₂O (0.5 mL) was added NaOH (21.95 mg, 548.67 μmol) at 0° C., the mixture was warmed to room temperature and stirred for 2 h. The reaction mixture was adjusted to pH 5 by addition aq. 10% citric acid (˜10 mL) and was then extracted with EtOAc (3×20 mL). The combined organic layers were washed with brine (2×5 mL), dried over Na₂SO₄, filtered and concentrated under reduced pressure to afford the desired product (92.6 mg, crude). LCMS (ESI) m/z: [M+H] calcd for C₁₁H₁₉NO₃S: 246.11; found 246.3.

### Intermediate A-77. Synthesis of (2R,3R)-1-((R)-tert-butylsulfinyl)-3-cyclobutylaziridine-2-carboxylic acid

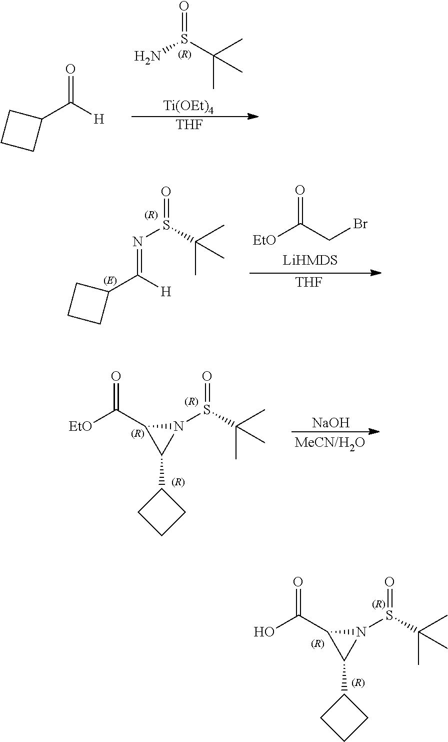

### Step 1: Synthesis of (R,E)-N-(cyclobutylmethylene)-2-methylpropane-2-sulfinamide

To a solution of cyclobutanecarbaldehyde (0.25 g, 2.97 mmol) in THF (5 mL) was added (R)-2-methylpropane-2-sulfinamide (396.24 mg, 3.27 mmol) and Ti(OEt)₄ (1.36 g, 5.94 mmol, 1.23 mL). The mixture was stirred at 75° C. for 3 h in two batches. The two batches were combined and the reaction mixture was quenched by the addition of brine (15 mL). The solution was extracted with EtOAc (3×20 mL) and the combined organic layers were washed with brine (2×5 mL), dried over Na₂SO₄, filtered and concentrated under reduced pressure to give a residue, which was purified by silica gel chromatography (10%→20% EtOAc/pet. ether) to afford the desired product (786.7 mg, 70.7% yield). LCMS (ESI) m/z: [M+H] calcd for C₉H₁₇NOS: 188.1; found 188.3.

### Step 2: Synthesis of ethyl (2R,3R)-1-((R)-tert-butylsulfinyl)-3-cyclobutylaziridine-2-carboxylate

To a solution of ethyl 2-bromoacetate (236.19 μL, 2.14 mmol) in THF (2 mL) was added LiHMDS (1 M, 2.14 mL) at −78° C., after 30 min, (R,E)-N-(cyclobutylmethylene)-2-methylpropane-2-sulfinamide (0.2 g, 1.07 mmol) was added. The mixture was warmed to −40° C. and stirred for 4 h. The reaction mixture was quenched by addition H₂O (18 mL) at −40° C. and warmed to room temperature The mixture was extracted with EtOAc (3×15 mL) and the combined organic layers were washed with brine (2×5 mL), dried over Na₂SO₄, filtered and concentrated under reduced pressure to give a residue, which was purified by prep-TLC (20% EtOAc/pet. ether) to afford the desired product (0.1 g, crude). LCMS (ESI) m/z: [M+H] calcd for C₁₃H₂₃NO₃S: 274.14; found 274.3.

### Step 3: Synthesis of (2R,3R)-1-((R)-tert-butylsulfinyl)-3-cyclobutylaziridine-2-carboxylic acid

In two batches, to a solution ethyl (2R,3R)-1-((R)-tert-butylsulfinyl)-3-cyclobutylaziridine-2-carboxylate (25 mg, 91.44 μmol) in MeCN (0.25 mL) and H₂O (0.25 mL) was added NaOH (5.49 mg, 137.17 μmol) at 0° C., the mixture was warmed to room temperature and stirred for 5 h. The reaction mixtures were combined, and adjust to pH to 5 with aq. 10% citric acid (10 mL), then extracted with EtOAc (3×20 mL). The combined organic layers were washed with brine (2×5 mL), dried over Na₂SO₄, filtered and concentrated under reduced pressure to afford the desired product (53 mg, crude). LCMS (ESI) m/z: [M+H] calcd for C₁₁H₁₉NO₃S: 246.11; found 246.2.

### Intermediate A-78. Synthesis of N-methyl-N-(methyl((S)-1-((R)-1-tritylaziridine-2-carbonyl)piperidin-3-yl)carbamoyl)-L-valine

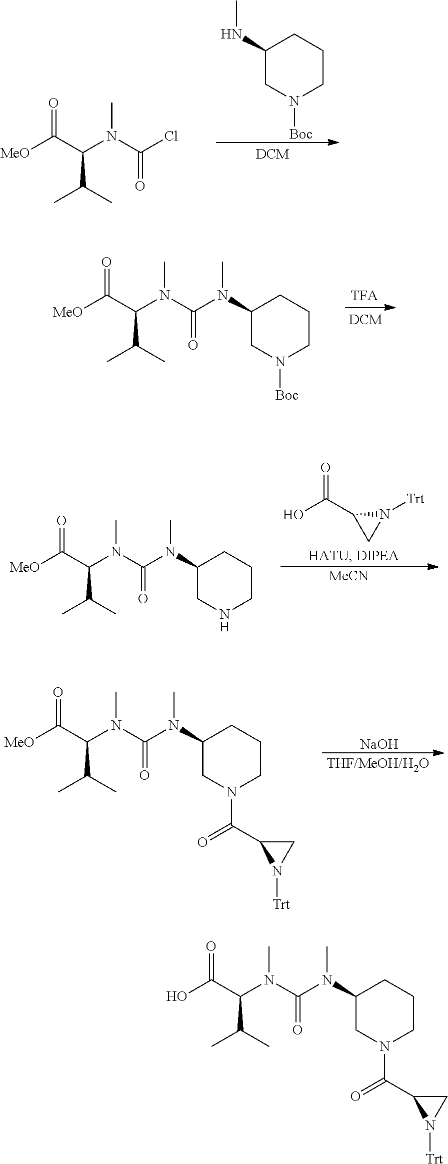

### Step 1: Synthesis of tert-butyl (S)-3-(3-((S)-1-methoxy-3-methyl-1-oxobutan-2-yl)-1,3-dimethylureido)piperidine-1-carboxylate

A mixture of methyl N-(chlorocarbonyl)-N-methyl-L-valinate (1.94 g, 9.34 mmol) in DCM was added to a solution of (S)-tert-butyl 3-(methylamino)piperidine-1-carboxylate (2.80 g, 13.08 mmol) in DCM (18 mL) at 0° C. The mixture was stirred at 40° C. for 3 h. The mixture was added to saturated aq. NH₄Cl (80 mL), and the aqueous phase was extracted with DCM (3×40 mL). the combined organic phase was washed with brine (50 mL), dried over Na₂SO₄, filtered and concentrated under reduced pressure. The crude product was purified by silica gel chromatography (30%→100% EtOAc/pet. ether) to afford the desired product (1.9 g, 55.3% yield). LCMS (ESI) m/z: [M+H] calculated for C₁₉H₃₆N₃O₅: 386.26; found 386.2.

### Step 2: Synthesis of methyl N-methyl-N-(methyl((S)-piperidin-3-yl)carbamoyl)-L-valinate

To a solution of give tert-butyl (S)-3-(3-((S)-1-methoxy-3-methyl-1-oxobutan-2-yl)-1,3-dimethylureido)piperidine-1-carboxylate (1 g, 2.59 mmol) in DCM (10 mL) was added TFA (3.84 mL, 51.88 mmol) at 0° C. The reaction was stirred at room temperature for 1 h. The mixture was added into saturated aq. Na₂CO₃ (100 mL) at 0° C. to adjust to pH 9. The aqueous phase was extracted with DCM (3 25×50 mL) and the combined organic phases were washed with brine (10 mL), dried with Na₂SO₄, filtered and concentrated under reduced pressure to afford the desired product (710 mg, crude). LCMS (ESI) m/z: [M+H] calculated for C₁₄H₂₇N₃O₃: 286.21; found 286.1.

### Step 3: Synthesis of methyl N-methyl-N-(methyl((S)-1-((R)-1-tritylaziridine-2-carbonyl)piperidin-3-yl)carbamoyl)-L-valinate

To a solution of (R)-1-tritylaziridine-2-carboxylic acid (1.24 g, 2.63 mmol, 70% purity) in MeCN (5 mL) at 0° C. was added DIPEA (1.22 mL, 7.01 mmol) and HATU (1.33 g, 3.50 mmol) followed by methyl N-methyl-N-(methyl((S)-piperidin-3-yl)carbamoyl)-L-valinate (500 mg, 1.75 mmol). The reaction mixture was warmed to room temperature and stirred 30 min. The mixture was added to saturated aq. NH₄Cl (100 mL) and the aqueous phase was extracted with DCM (3×50 mL). The combined organic phase was washed with brine (60 mL), dried over Na₂SO₄, filtered and concentrated under reduced pressure. The residue was purified by silica gel chromatography (50%→100% EtOAc/pet. ether) to afford the desired product (650 mg, 49.7% yield). LCMS (ESI) m/z: [M+H] calculated for C₃₆H₄₄N₄O₄: 597.34; found 597.3.

### Step 4: Synthesis of N-methyl-N-(methyl((S)-1-((R)-1-tritylaziridine-2-carbonyl)piperidin-3-yl)carbamoyl)-L-valine

NaOH (58.34 mg, 1.46 mmol) was added to a solution of methyl N-methyl-N-(methyl((S)-1-((R)-1-tritylaziridine-2-carbonyl)piperidin-3-yl)carbamoyl)-L-valinate (640 mg, 857.97 μmol) in THF (4 mL), MeOH (1.3 mL), and H₂O (1.3 mL). The mixture was stirred at room temperature for 20 h. The reaction solution was directly lyophilized to afford the desired product (700 mg, crude). LCMS (ESI) m/z: [M+H] calculated for C₃₅H₄₂N₄O₄: 583.32; found 583.4.

### Intermediate A-79. Synthesis of N-methyl-N-(methyl((S)-1-((R)-1-tritylaziridine-2-carbonyl)pyrrolidin-3-yl)carbamoyl)-L-valine

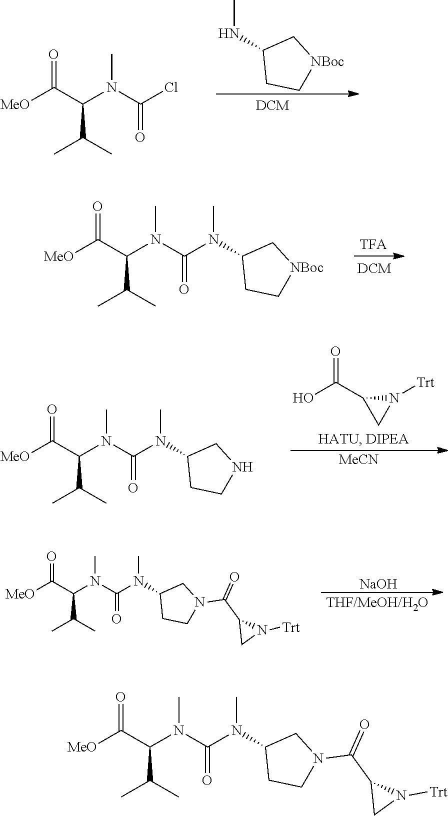

### Step 1: Synthesis of tert-butyl (S)-3-(3-((S)-1-methoxy-3-methyl-1-oxobutan-2-yl)-1,3-dimethylureido)pyrrolidine-1-carboxylate

A solution of methyl N-(chlorocarbonyl)-N-methyl-L-valinate (1.14 g, 5.49 mmol) in DCM (10 mL) was added to a solution of tert-butyl (S)-3-(methylamino)pyrrolidine-1-carboxylate (1.54 g, 7.69 mmol) in DCM (10 mL) at 0° C. The mixture was warmed to room temperature and stirred for 2 h. The mixture was then added to sat. NH₄Cl (50 mL), and the aqueous phase was extracted with DCM (3×30 mL). The combined organic phase was washed with brine (30 mL), dried over Na₂SO₄, filtered and concentrated under reduced pressure. The crude product was purified by silica gel chromatography (30%→100% EtOAc/pet. ether) to afford the desired product (1.07 g, 52.5% yield).

### Step 2: Synthesis of methyl N-methyl-N-(methyl((S)-pyrrolidin-3-yl)carbamoyl)-L-valinate

To a solution of tert-butyl (S)-3-(3-((S)-1-methoxy-3-methyl-1-oxobutan-2-yl)-1,3-dimethylureido)pyrrolidine-1-carboxylate (1.05 g, 2.83 mmol) in DCM (11 mL) at 0° C. was added TFA (4.19 mL, 56.53 mmol). The reaction was then warmed to room temperature and stirred for 1 h. The mixture was added to sat. Na₂CO₃ (200 mL) at 0° C. dropwise to adjust to pH 9. The aqueous phase was extracted with DCM (3×100 mL), and the combined organic phase was washed with brine (100 mL), dried with Na₂SO₄, filtered and concentrated under reduced pressure to afford the desired product (800 mg, crude).

### Step 3: Synthesis of methyl N-methyl-N-(methyl((S)-1-((R)-1-tritylaziridine-2-carbonyl)pyrrolidin-3-yl)carbamoyl)-L-valinate

To a solution of (R)-1-tritylaziridine-2-carboxylic acid (1.04 g, 2.21 mmol) in MeCN (4 mL) at 0° C. was added HATU (1.12 g, 2.95 mmol), and DIPEA (1.03 mL, 5.90 mmol) followed by methyl N-methyl-N-(methyl((S)-pyrrolidin-3-yl)carbamoyl)-L-valinate (400 mg, 1.47 mmol). The mixture was warmed to room temperature and stirred for 0.5 h. The mixture was poured into NH₄Cl aq. (50 mL) and extracted with DCM (3×20 mL). The combined organic phases were washed with brine (30 mL), dried with Na₂SO₄, filtered and concentrated under reduced pressure. The crude product was purified by silica gel chromatography (50%→100% EtOAc/pet. ether) to afford the desired product (580 mg, 67.5% yield).

### Step 4: Synthesis of N-methyl-N-(methyl((S)-1-((R)-1-tritylaziridine-2-carbonyl)pyrrolidin-3-yl)carbamoyl)-L-valine

To a solution of methyl N-methyl-N-(methyl((S)-1-((R)-1-tritylaziridine-2-carbonyl)pyrrolidin-3-yl)carbamoyl)-L-valinate (650 mg, 1.12 mmol) in THF (3.9 mL) and MeOH (1.3 mL) was added a solution of NaOH (89.23 mg, 2.23 mmol) in H₂O (1.3 mL). The mixture was stirred at room temperature for 16 h. The reaction mixture was diluted with H₂O (10 mL) and then lyophilized directly to afford the desired product (700 mg, crude). LCMS (ESI) m/z: [M+H] calcd for C₃₄H₄₀N₄O₄: 569.30; found, 569.4.

### Intermediate A-80. Synthesis of N-methyl-N—((S)-3-methyl-4-((R)-1-tritylaziridine-2-carbonyl)piperazine-1-carbonyl)-L-valine

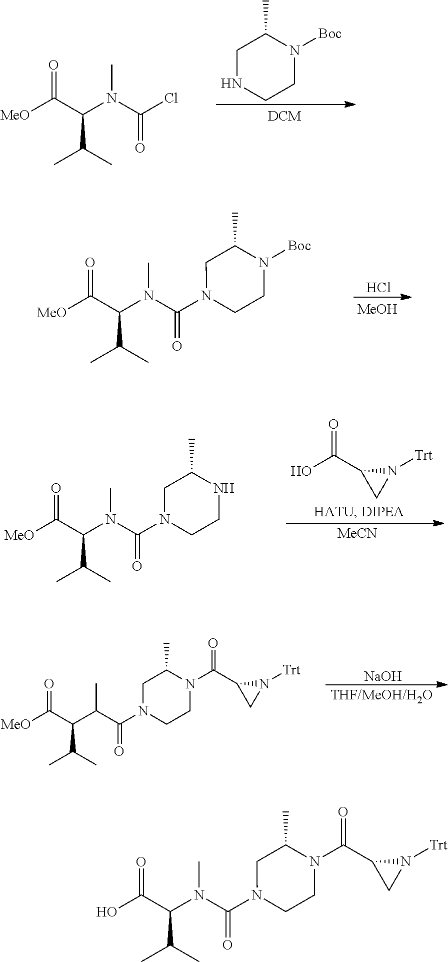

### Step 1: Synthesis of tert-butyl (S)-4-(((S)-1-methoxy-3-methyl-1-oxobutan-2-yl)(methyl)carbamoyl)-2-methylpiperazine-1-carboxylate

To a solution of methyl tert-butyl (S)-2-methylpiperazine-1-carboxylate (3.31 g, 16.52 mmol) in DCM (30 mL) at 0° C. was added a solution of methyl N-(chlorocarbonyl)-N-methyl-L-valinate in DCM (0.55 M, 30 mL). The mixture was stirred at room temperature for 30 min. The reaction mixture was diluted with H₂O (30 mL) and extracted with DCM (3×20 mL). The combined organic layers were washed with brine (2×15 mL), dried over Na₂SO₄, filtered and concentrated under reduced pressure to give a residue. The residue was purified by silica gel chromatography (20%→50% EtOAc/pet. ether) to afford the desired product (5 g, 81.5% yield). LCMS (ESI) m/z: [M+H] calcd for C₁₈H₃₃N₃O₅:372.2; found 372.1.

### Step 2: Synthesis of methyl N-methyl-N—((S)-3-methylpiperazine-1-carbonyl)-L-valinate

To tert-butyl (S)-4-(((S)-1-methoxy-3-methyl-1-oxobutan-2-yl)(methyl)carbamoyl)-2-methylpiperazine-1-carboxylate (3 g, 8.08 mmol) was added a solution of 4M HCl in MeOH (30 mL). The mixture was stirred at room temperature for 1 h. The reaction mixture was adjusted to pH 8 with saturated aq. NaHCO₃, and was then diluted with H₂O (50 mL) and extracted with DCM (3×30 mL). The combined organic layers were washed with brine (50 mL), dried over Na₂SO₄, filtered and concentrated under reduced pressure to afford the desired product (1.8 g, 82.1% yield).

### Step 3: Synthesis of methyl N-methyl-N—((S)-3-methyl-4-((R)-1-tritylaziridine-2-carbonyl)piperazine-1-carbonyl)-L-valinate

To a solution of (2R)-1-tritylaziridine-2-carboxylic acid (971.10 mg, 2.95 mmol) in MeCN (10 mL) was added HATU (1.35 g, 3.54 mmol), DIPEA (1.54 mL, 8.84 mmol) and methyl N-methyl-N—((S)-3-methylpiperazine-1-carbonyl)-L-valinate (0.8 g, 2.95 mmol). The mixture was stirred at room temperature for 12 h. The reaction mixture was then diluted with H₂O (20 mL) and extracted with DCM (3×15 mL). The combined organic layers were washed with brine 20 mL (2×10 mL), dried over Na₂SO₄, filtered and concentrated under reduced pressure to give a residue. The residue was purified by silica gel chromatography (30%-*50% EtOAc/pet. ether) to afford the desired product (0.35 g, 20.4% yield). LCMS (ESI) m/z: [M+H] calculated for C₃₅H₄₂N₄O₄: 583.3; found 583.2.

### Step 4: Synthesis of N-methyl-N—((S)-3-methyl-4-((R)-1-tritylaziridine-2-carbonyl)piperazine-1-carbonyl)-L-valine

To a solution of methyl N-methyl-N—((S)-3-methyl-4-((R)-1-tritylaziridine-2-carbonyl)piperazine-1-carbonyl)-L-valinate (200 mg, 343.21 μmol) in H₂O (1 mL), THF (1 mL), and MeOH (1 mL) at 0° C. was added LiOH·H₂O (14.40 mg, 343.21 μmol). The mixture was stirred at room temperature for 8 h and was then lyophilized directly to afford the desired product (390 mg, 98.7% yield). LCMS (ESI) m/z: [M+Na] calculated for C₃₄H₄₀N₄O₄: 591.3; found 591.2.

### Intermediate A-81. Synthesis of N-methyl-N—((S)-3-methyl-4-((S)-1-tritylaziridine-2-carbonyl)piperazine-1-carbonyl)-L-valine

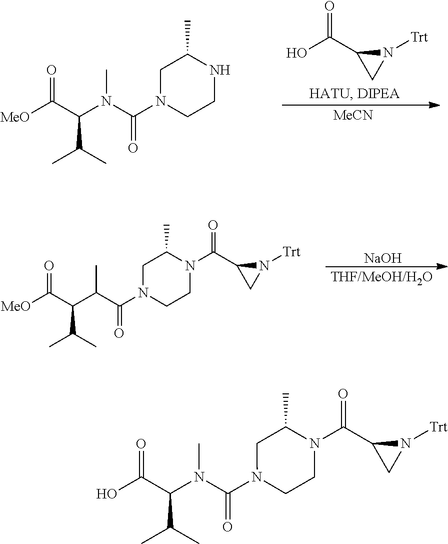

### Step 1: Synthesis of methyl N-methyl-N—((S)-3-methyl-4-((S)-1-tritylaziridine-2-carbonyl)piperazine-1-carbonyl)-L-valinate

To a solution of methyl N-methyl-N—((S)-3-methylpiperazine-1-carbonyl)-L-valinate (500 mg, 1.84 mmol) in MeCN (5 mL) at 0° C. was added (S)-1-tritylaziridine-2-carboxylic acid (1.30 g, 2.76 mmol, 70% purity), HATU (1.05 g, 2.76 mmol) and DIPEA (962.85 μL, 5.53 mmol). The mixture was stirred at room temperature for 30 min. The reaction mixture was then diluted with H₂O (10 mL) and extracted with DCM (3×5 mL). The combined organic layers were washed with brine (2×5 mL), dried over Na₂SO₄, filtered and concentrated under reduced pressure to give a residue. The residue was purified by silica gel chromatography (20%→33% EtOAc/pet. ether) to afford the desired product (0.5 g, 46.6% yield). LCMS (ESI) m/z: [M+Na] calculated for C₃₅H₄₂O₄N₄: 605.2; found 605.2.

### Step 2: Synthesis of N-methyl-N—((S)-3-methyl-4-((S)-1-tritylaziridine-2-carbonyl)piperazine-1-carbonyl)-L-valine

To a solution of methyl N-methyl-N—((S)-3-methyl-4-((S)-1-tritylaziridine-2-carbonyl)piperazine-1-carbonyl)-L-valinate (400 mg, 686.42 μmol) in H₂O (2 mL), THF (2 mL), and MeOH (2 mL) at 0° C. was added LiOH·H₂O (28.80 mg, 686.42 μmol). The mixture was stirred at room temperature for 8 h. The mixture was lyophilized directly to afford then desired product (390 mg, 98.7% yield). LCMS (ESI) m/z: [M+Na] calculated for C₃₄H₄₀N₄O₄: 591.3; found 591.2.

### Intermediate A-82. Synthesis of N-methyl-N—((R)-3-methyl-4-((R)-1-tritylaziridine-2-carbonyl)piperazine-1-carbonyl)-L-valine

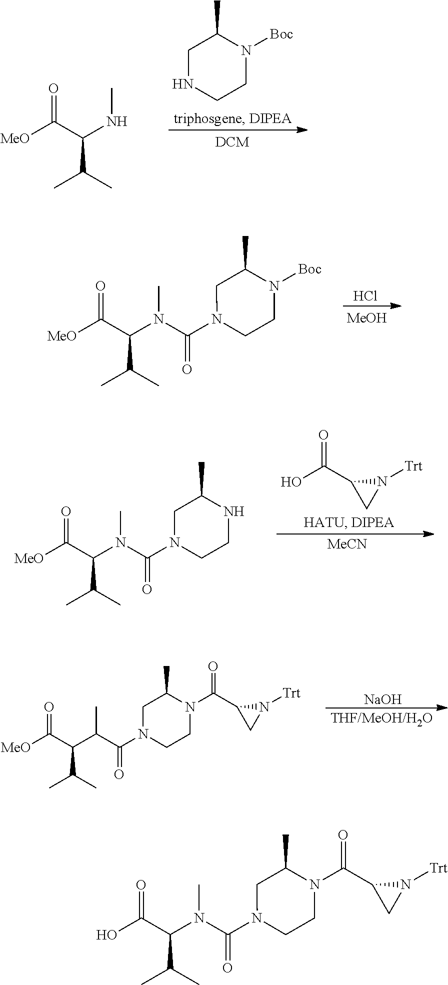

### Step 1: Synthesis of tert-butyl (R)-4-(((S)-1-methoxy-3-methyl-1-oxobutan-2-yl)(methyl)carbamoyl)-2-methylpiperazine-1-carboxylate

To a mixture of methyl methyl-L-valinate hydrochloride (3 g, 16.51 mmol) and DIPEA (17.26 mL, 99.09 mmol) in DCM (60 mL) at 0° C. was added bis(trichloromethyl) carbonate (2.45 g, 8.26 mmol) in one portion. The mixture was stirred at 0° C. for 30 min, then tert-butyl (R)-2-methylpiperazine-1-carboxylate (3.31 g, 16.51 mmol) was added to the mixture. The mixture was stirred at 0° C. for 1 h, then the pH of the solution was adjusted to 8 with sat. NaHCO₃. The residue was poured into H₂O (20 mL) and stirred for 5 min. The aqueous phase was extracted with EtOAc (2×20 mL), and the combined organic phase was washed with sat. NaHCO₃ (20 mL), dried with Na₂SO₄, filtered and concentrated under reduced pressure. The residue was purified by silica gel chromatography (1%→10% EtOAc/pet. ether) to afford the desired product (3.1 g, 50.5% yield). LCMS (ESI) m/z: [M+H] calculated for C₁₈H₃₃N₃O₅: 372.3; found 372.2.

### Step 2: Synthesis of methyl N-methyl-N—((R)-3-methylpiperazine-1-carbonyl)-L-valinate

To a mixture of tert-butyl (R)-4-(((S)-1-methoxy-3-methyl-1-oxobutan-2-yl)(methyl)carbamoyl)-2-methylpiperazine-1-carboxylate (2.5 g, 6.73 mmol) was added 4M HCl in MeOH (25 mL) at 0° C. The mixture was stirred at room temperature for 1 h. The mixture was then concentrated under reduced pressure to afford the desired product (2 g, 96.5% yield).

### Step 3: Synthesis of methyl N-methyl-N—((R)-3-methyl-4-((R)-1-tritylaziridine-2-carbonyl)piperazine-1-carbonyl)-L-valinate

To a mixture of (R)-1-tritylaziridine-2-carboxylic acid (1.03 g, 3.12 mmol) and HATU (1.11 g, 2.92 mmol) in MeCN (1 mL) was added DIPEA (1.36 mL, 7.80 mmol) followed by methyl N-methyl-N—((R)-3-methylpiperazine-1-carbonyl)-L-valinate (600 mg, 1.95 mmol). The mixture was stirred at room temperature for 1 h. The mixture was then concentrated under reduced pressure. The residue was purified by silica gel chromatography (1%→50% EtOAc/pet. ether) to afford the desired product (450 mg, 39.62% yield). LCMS (ESI) m/z: [M+H] calculated for C₃₅H₄₂N₄O₄: 583.3; found 583.2.

### Step 4: Synthesis of N-methyl-N—((R)-3-methyl-4-((R)-1-tritylaziridine-2-carbonyl)piperazine-1-carbonyl)-L-valine

To a mixture of methyl N-methyl-N—((R)-3-methyl-4-((R)-1-tritylaziridine-2-carbonyl)piperazine-1-carbonyl)-L-valinate (450 mg, 772.23 μmol) in H₂O (1 mL), MeOH (1 mL), and THF (3 mL) was added LiOH·H₂O (48.60 mg, 1.16 mmol). The mixture was stirred at room temperature for 10 h and was then lyophilized to afford the desired product (410 mg, 92.4% yield). LCMS (ESI) m/z: [M+Na] calculated for C₃₄H₄₀N₄O₄: 591.3; found 591.3.

### Intermediate A-83. Synthesis of N-methyl-N—((R)-3-methyl-4-((S)-1-tritylaziridine-2-carbonyl)piperazine-1-carbonyl)-L-valine

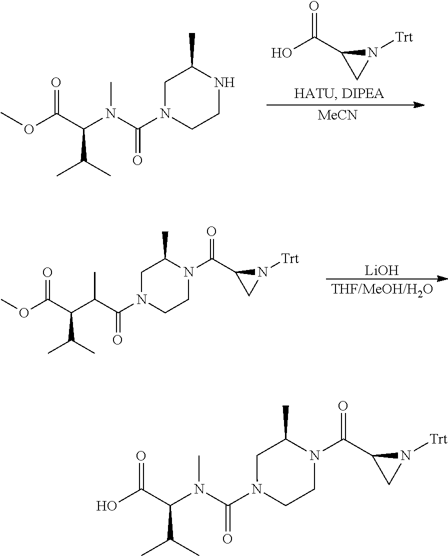

### Step 1: Synthesis of methyl N-methyl-N—((R)-3-methyl-4-((S)-1-tritylaziridine-2-carbonyl)piperazine-1-carbonyl)-L-valinate

To a mixture of (S)-1-tritylaziridine-2-carboxylic acid (1.03 g, 3.12 mmol) and HATU (1.11 g, 2.92 mmol) in MeCN (1 mL) was added DIPEA (1.36 mL, 7.80 mmol) followed by methyl N-methyl-N—((R)-3-methylpiperazine-1-carbonyl)-L-valinate (600 mg, 1.95 mmol). The mixture was stirred at room temperature for 1 h and was then concentrated under reduced pressure. The residue was purified by silica gel chromatography (0%→50% EtOAc/pet. ether) to afford the desired product (430 mg, 37.8% yield). LCMS (ESI) m/z: [M+H] calculated for C₃₅H₄₂N₄O₄: 583.3; found 583.2.

### Step 2: Synthesis of N-methyl-N—((R)-3-methyl-4-((S)-1-tritylaziridine-2-carbonyl)piperazine-1-carbonyl)-L-valine

To a mixture of methyl N-methyl-N—((R)-3-methyl-4-((S)-1-tritylaziridine-2-carbonyl)piperazine-1-carbonyl)-L-valinate (430 mg, 737.91 μmol) in H₂O (1 mL), MeOH (1 mL), and THF (3 mL) was added LiOH·H₂O (46.44 mg, 1.11 mmol). The mixture was stirred at room temperature for 10 h and was then lyophilized to afford the desired product (370 mg, 87.3% yield). LCMS (ESI) m/z: [M+Na] calculated for C₃₄H₄₀N₄O₄: 591.3; found 591.3.

### Intermediate A-84. Synthesis of N—((R)-4-(tert-butoxycarbonyl)-2-methylpiperazine-1-carbonyl)-N-methyl-L-valine

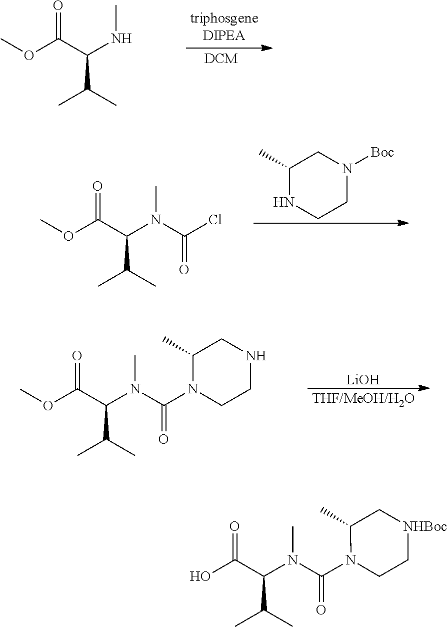

### Step 1: Synthesis of methyl N-(chlorocarbonyl)-N-methyl-L-valinate

To a solution of methyl methyl-L-valinate hydrochloride (1.8 g, 9.91 mmol) in DCM (20 mL) at 0° C. was added DIPEA (5.18 mL, 29.73 mmol) followed by bis(trichloromethyl) carbonate (1.47 g, 4.95 mmol). The mixture was stirred at 0° C. for 20 min. The reaction mixture used for the next step directly without workup.

Step 2: Synthesis of tert-butyl (R)-4-(((S)-1-methoxy-3-methyl-1-oxobutan-2-yl)(methyl)carbamoyl)-3-methylpiperazine-1-carboxylate

A solution of methyl N-(chlorocarbonyl)-N-methyl-L-valinate (1.03 g, 4.96 mmol) in DCM (10 mL) was added to a solution of tert-butyl (3R)-3-methylpiperazine-1-carboxylate (993.41 mg, 4.96 mmol) in DCM (1 mL) at 0° C. The mixture was then stirred at 0° C. for 15 min. The mixture was added to aq. NH₄Cl (10 mL) and the solution was then extracted with DCM (3×10 mL). The combined organic phase was washed with brine (2 mL), dried over Na₂SO₄, filtered and concentrated under reduced pressure. The residue was purified by silica gel chromatography (0%→50% EtOAc/pet. ether) to afford the desired product (750 mg, 36.2% yield).

### Step 3: Synthesis of N—((R)-4-(tert-butoxycarbonyl)-2-methylpiperazine-1-carbonyl)-N-methyl-L-valine

To a solution of tert-butyl (R)-4-(((S)-1-methoxy-3-methyl-1-oxobutan-2-yl)(methyl)carbamoyl)-3-methylpiperazine-1-carboxylate (700 mg, 1.88 mmol) in THF (0.5 mL) and H₂O (0.5 mL) at 0° C. was added LiOH·H₂O (237.23 mg, 5.65 mmol). The mixture was stirred at room temperature for 3 h. The pH of the reaction mixture was adjusted to 6-7 with 1 N HCl. The mixture was extracted with EtOAc (3×10 mL), dried over Na₂SO₄, and concentrated under reduced pressure to afford the desired product (600 mg, 85.5% yield).

### Intermediate A-85. Synthesis of (S)-3-methyl-2-((R)-2-oxo-3-((S)-1-tritylaziridine-2-carboxamido)pyrrolidin-1-yl)butanoic acid

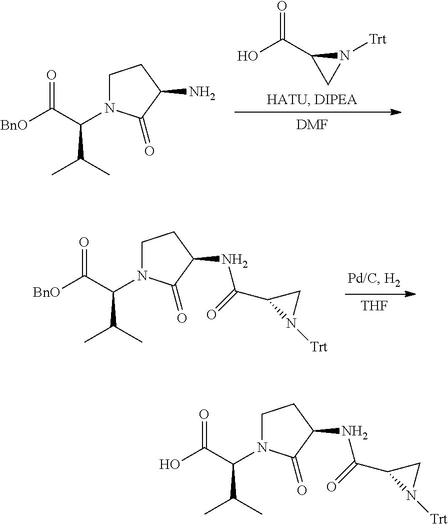

### Step 1: Synthesis of benzyl (S)-3-methyl-2-((R)-2-oxo-3-((S)-1-tritylaziridine-2-carboxamido)pyrrolidin-1-yl)butanoate

To a mixture of benzyl (2S)-2-[(3R)-3-amino-2-oxopyrrolidin-1-yl]-3-methylbutanoate (420.0 mg, 1.446 mmol), DIPEA (934.73 mg, 7.232 mmol) and (2S)-1-(triphenylmethyl)aziridine-2-carboxylic acid (619.40 mg, 1.880 mmol) in DMF (5 mL) at 0° C. was added HATU (659.99 mg, 1.736 mmol). The resulting mixture was stirred at room temperature for 2 h. The reaction mixture was quenched with H₂O. The resulting mixture was extracted with EtOAc (2×10 mL), and the combined organic layers were washed with brine (10 mL), dried over Na₂SO₄, filtered, and concentrated under reduced pressure. The residue was purified by silica gel column chromatography (30% EtOAc/pet. ether) to afford the desired product (480 mg, 55.2% yield). LCMS (ESI) m/z: [M−H]⁻ calcd for C₃₈H₃₈N₃O₄: 600.29; found 600.3.

### Step 2: Synthesis of (S)-3-methyl-2-((R)-2-oxo-3-((S)-1-tritylaziridine-2-carboxamido)pyrrolidin-1-yl)butanoic acid

A suspension of benzyl (2S)-3-methyl-2-[(3R)-2-oxo-3-[(2S)-1-(triphenylmethyl)aziridine-2-amido]pyrrolidin-1-yl]butanoate (450.0 mg, 0.748 mmol) and Pd/C (200 mg) in THF (5 mL) at room temperature was stirred for 3 h under a hydrogen atmosphere. The mixture was then filtered and concentrated under reduced pressure to afford the desired product (400 mg, crude). LCMS (ESI) m/z: [M−H] calcd for C₃₁H₃₂N₃O₄: 510.24; found 510.2.

### Intermediate A-86. Synthesis of (S)-2-(8-(tert-butoxycarbonyl)-1-oxo-2,8-diazaspiro[4.5]decan-2-yl)-3-methylbutanoic acid

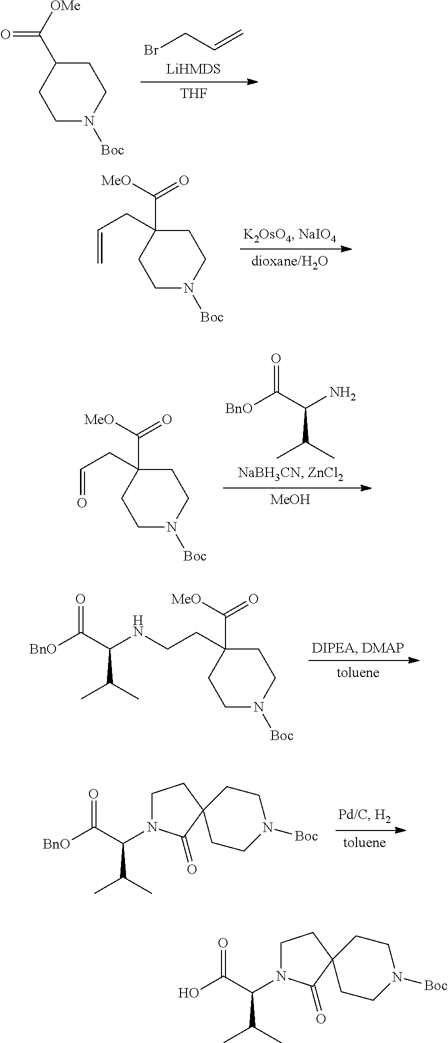

### Step 1: Synthesis of 1-(tert-butyl) 4-methyl 4-allylpiperidine-1,4-dicarboxylate

To a solution of 1-tert-butyl 4-methyl piperidine-1,4-dicarboxylate (5.0 g, 20.551 mmol) in THF (50 mL) at −78° C. was added LiHMDS (27 mL, 26.714 mmol, 1M in THF) followed by allyl bromide (3.23 g, 26.716 mmol). The resulting mixture was stirred overnight at room temperature. The reaction was quenched with sat. aq. NH₄Cl (aq.) and the combined organic layers were washed with brine (3×100 mL), dried over Na₂SO₄, filtered and concentrated under reduced pressure. The residue was purified by silica gel column chromatography (50% EtOAc/pet. ether) to afford the desired product (4.5 g, 73.4% yield).

### Step 2: Synthesis of 1-(tert-butyl) 4-methyl 4-(2-oxoethyl)piperidine-1,4-dicarboxylate

To a solution of 1-tert-butyl 4-methyl 4-(prop-2-en-1-yl)piperidine-1,4-dicarboxylate (1.0 g, 3.529 mmol) and K₂OsO₄·2H₂O (1.3 g, 3.529 mmol) in 1,4-dioxane (5 mL) and H₂O (5 mL) at 0° C. was added NaIO₄ (1.51 g, 7.058 mmol). The resulting mixture was stirred at room temperature for 5 h. The mixture was extracted with EtOAc (3×50 mL) and the combined organic layers were washed with H₂O (3×100 mL), dried over Na₂SO₄, filtered, and concentrated under reduced pressure. The crude product was used in the next step directly without further purification to afford the desired product (800 mg, 75.% yield). LCMS (ESI) m/z: [M−H] calcd for C₁₄H₂₃NO₅: 284.16; found 284.0.

### Step 3: Synthesis of 1-(tert-butyl) 4-methyl (S)-4-(2-((1-(benzyloxy)-3-methyl-1-oxobutan-2-yl)amino)ethyl)piperidine-1,4-dicarboxylate

To a solution of 1-tert-butyl 4-methyl 4-(2-oxoethyl)piperidine-1,4-dicarboxylate (4.0 g, 14.018 mmol) and benzyl (2S)-2-amino-3-methylbutanoate (3.49 g, 16.822 mmol) in MeOH (40 mL) at 0° C. was added ZnCl₂ (2.10 g, 15.420 mmol) and NaBH₃CN (1.76 g, 28.037 mmol). The resulting mixture was stirred at room temperature for 2 h. The reaction was quenched with sat. aq. NH₄Cl and the combined organic layers were washed with brine (3×100 mL), dried over Na₂SO₄, filtered, and concentrated under reduced pressure. The residue was purified by silica gel column chromatography (50% EtOAc/pet. ether) to afford the desired product. LCMS (ESI) m/z: [M+H] calcd for C₂₆H₄₀N₂O₆: 477.29; found 477.3.

### Step 4: Synthesis of tert-butyl (S)-2-(1-(benzyloxy)-3-methyl-1-oxobutan-2-yl)-1-oxo-2,8-diazaspiro[4.5]decane-8-carboxylate

To a solution of 1-tert-butyl 4-methyl 4-(prop-2-en-1-yl)piperidine-1,4-dicarboxylate (2.20 g, 4.616 mmol) and DIPEA (5.97 g, 46.159 mmol) in toluene was added DMAP (0.56 g, 4.616 mmol) in portions at 120° C. The resulting mixture was stirred overnight at 120° C. The reaction was cooled to room temperature and quenched with sat. aq. NH₄Cl. The combined organic layers were washed with brine (3×100 mL), dried over Na₂SO₄, filtered, and concentrated under reduced pressure. The residue was purified by Prep-TLC (50% EtOAc/pet. ether) to afford the desired product (1.5 g, 50.2% yield). LCMS (ESI) m/z: [M+H] calcd for C₂₅H₃₆N₂O₅: 445.26; found 445.3.

### Step 5: Synthesis of (S)-2-(8-(tert-butoxycarbonyl)-1-oxo-2,8-diazaspiro[4.5]decan-2-yl)-3-methylbutanoic acid

To a solution of tert-butyl 2-[(2S)-1-(benzyloxy)-3-methyl-1-oxobutan-2-yl]-1-oxo-2,8-diazaspiro[4.5]decane-8-carboxylate (2.40 g, 5.398 mmol) in toluene (25 mL) at room temperature was added Pd/C (2.40 g, 22.552 mmol). The resulting suspension was stirred overnight at room temperature under an H₂ atmosphere. The mixture was concentrated under reduced pressure, filtered, the filter cake washed with EtOAc (3×50 mL), and the filtrate was concentrated under reduced pressure. The residue was purified by silica gel column chromatography (50% EtOAc/pet. ether) to afford the desired product (2.2 g, 72.5% yield). LCMS (ESI) m/z: [M−H] calcd for C₁₈H₃₀N₂O₅: 353.22; found 353.2.

### Intermediate A-87. Synthesis of N-methyl-N-(3-oxo-4-((S)-1-tritylaziridine-2-carbonyl)piperazine-1-carbonyl)-L-valine

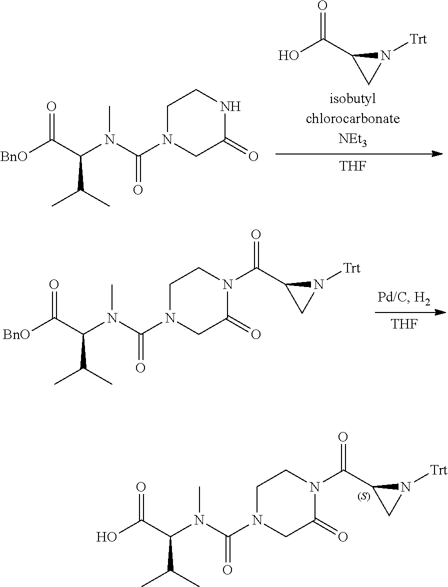

### Step 1: Synthesis of benzyl N-methyl-N-(3-oxo-4-((S)-1-tritylaziridine-2-carbonyl)piperazine-1-carbonyl)-L-valinate

To a solution of (2S)-1-(triphenylmethyl)aziridine-2-carboxylic acid (2.13 g, 6.466 mmol) in THF (10 mL) at 0° C. was added Et₃N (0.87 g, 8.598 mmol) and isobutyl chlorocarbonate (1.44 g, 10.54 mmol). The resulting mixture was stirred at room temperature for 1 h, and then benzyl (2S)-3-methyl-2-[methyl(3-oxopiperazine-1-carbonyl)amino]butanoate (1.50 g, 4.318 mmol) was added. The resulting mixture was stirred overnight at 70° C. The reaction mixture was then concentrated under reduced pressure. The residue was purified by Prep-TLC (50% EtOAc/pet. ether) to afford the desired product (900 mg, 31.6% yield). LCMS (ESI) m/z: [M−H] calcd for C₄₀H₄₂N₄O₅: 657.32; found 657.1.

### Step 2: Synthesis of N-methyl-N-(3-oxo-4-((S)-1-tritylaziridine-2-carbonyl)piperazine-1-carbonyl)-L-valine

A solution of benzyl N-methyl-N-(3-oxo-4-((S)-1-tritylaziridine-2-carbonyl)piperazine-1-carbonyl)-L-valinate (500 mg) and Pd/C (50 mg) in THF (5 mL) was stirred for 2 h at room temperature under a hydrogen atmosphere. The resulting mixture was filtered, the filter cake was washed with MeOH (3×30 mL), and the filtrate was concentrated under reduced pressure to afford the desired product (460 mg, crude). LCMS (ESI) m/z: [M−H] calcd for C₃₃H₃₆N₄O₅: 567.27; found 567.1.

### Intermediate A-88. Synthesis of lithium (R)-1-(3-methoxypropyl)aziridine-2-carboxylate

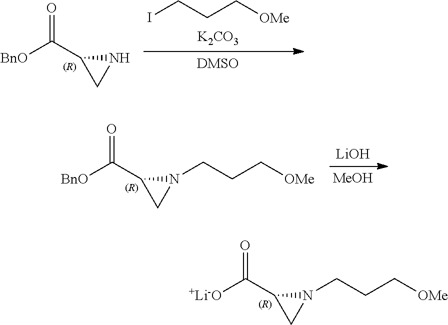

### Step 1: Synthesis of benzyl (R)-1-(3-methoxypropyl)aziridine-2-carboxylate

To a mixture of benzyl (R)-aziridine-2-carboxylate (350.0 mg, 1.975 mmol) and K₂CO₃ (545.95 mg, 3.950 mmol) in DMSO (4 mL) at 60° C. was added 1-iodo-3-methoxypropane (790.13 mg, 3.950 mmol). The resulting mixture was stirred for 2 h and was then cooled to room temperature, diluted with brine (50 mL), and extracted with EtOAc (3×20 mL). The combined organic layers were concentrated under reduced pressure. The crude product was purified by reverse phase chromatography (30%→38% MeCN/H₂O) to afford the desired product (170 mg, 31.1% yield). LCMS (ESI) m/z: [M+H] calcd for C₁₄H₁₉NO₃: 250.14; found 250.2.

### Step 2: Synthesis of lithium (R)-1-(3-methoxypropyl)aziridine-2-carboxylate

A mixture of benzyl (R)-1-(3-methoxypropyl)aziridine-2-carboxylate (170 mg, 0.682 mmol) and LiOH (57.23 mg, 1.364 mmol) in MeOH (2 mL) was stirred at 0° C. for 1 h. The mixture was concentrated under reduced pressure to afford the desired product (200 mg, crude). LCMS (ESI) m/z: [M+H] calcd for C₇H₁₃NO₃: 160.09; found 160.3.

### Intermediate A-89. Synthesis of lithium (S)-1-(3-methoxypropyl)aziridine-2-carboxylate

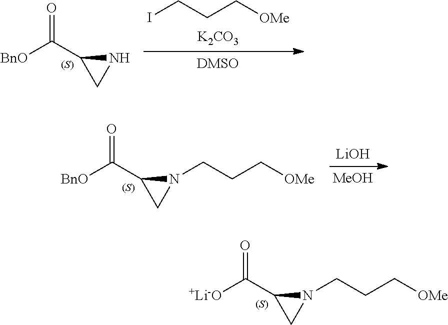

### Step 1: Synthesis of benzyl (S)-1-(3-methoxypropyl)aziridine-2-carboxylate

To a mixture of benzyl (S)-aziridine-2-carboxylate (250 mg, 1.411 mmol) and K₂CO₃ (389.96 mg, 2.822 mmol) in DMSO (4 mL) at 60° C. was added 1-iodo-3-methoxypropane (564.38 mg, 2.822 mmol). The resulting mixture was stirred for 2 h and was then cooled to room temperature, diluted with brine (50 mL), and extracted with EtOAc (3×20 mL). The combined organic layers were concentrated under reduced pressure. The crude product was purified by reverse phase chromatography (25%→40% H₂O/MeCN) to afford the desired product (234 mg, 63.2% yield). LCMS (ESI) m/z: [M+H] calcd for C₁₄H₁₉NO₃: 250.14; found 250.2.

### Step 2: Synthesis of lithium (S)-1-(3-methoxypropyl)aziridine-2-carboxylate

A mixture of benzyl (S)-1-(3-methoxypropyl) aziridine-2-carboxylate (230 mg, 0.923 mmol) and LiOH·H₂O (77.43 mg, 1.845 mmol) in MeOH (3 mL) was stirred for 1 h at 0° C. The resulting mixture was concentrated under reduced pressure to afford the desired product (320 mg, crude). LCMS (ESI) m/z: [M+H] calcd for C₇H₁₃NO₃: 160.09; found 160.1.

### Intermediate A-90. Synthesis of tert-butyl (S)-2-((S)-1-(benzyloxy)-3-methyl-1-oxobutan-2-yl)-1-oxo-2,7-diazaspiro[4.5]decane-7-carboxylate and tert-butyl (R)-2-((S)-1-(benzyloxy)-3-methyl-1-oxobutan-2-yl)-1-oxo-2,7-diazaspiro[4.5]decane-7-carboxylate

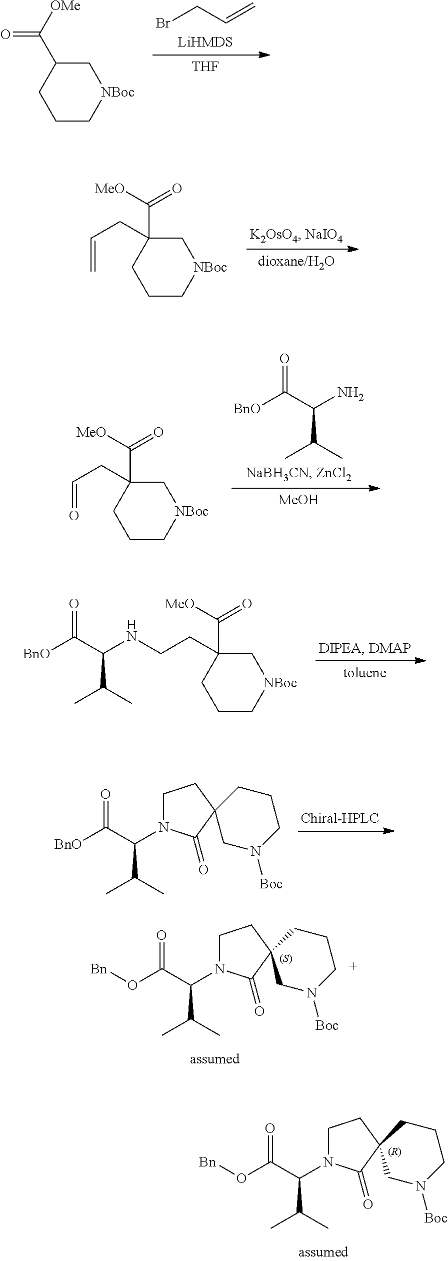

### Step 1: Synthesis of 1-(tert-butyl) 3-methyl 3-allylpiperidine-1,3-dicarboxylate

To a solution of 1-tert-butyl 3-methyl piperidine-1,3-dicarboxylate (10.0 g, 41.101 mmol) and LiHMDS (82 mL, 82.202 mmol, 1M in THF) in THF (100 mL) at −78° C. was added allyl bromide (9.94 g, 82.202 mmol). The reaction was warmed to room temperature and stirred overnight. The solution was then quenched with sat. aq. NH₄Cl and diluted with EtOAc (500 mL). The organic layer was washed with brine (3×150 mL), dried over Na₂SO₄, filtered and concentrated under reduced pressure. The residue was purified by silica gel column chromatography to afford the desired product (9.9 g, 85% yield). LCMS (ESI) m/z: [M+H] calcd for C₁₅H₂₅NO₄: 284.18; found 284.0.

### Step 2: Synthesis of 1-(tert-butyl) 3-methyl 3-(2-oxoethyl)piperidine-1,3-dicarboxylate

To a solution of 1-(tert-butyl) 3-methyl 3-allylpiperidine-1,3-dicarboxylate1-(tert-butyl) 3-methyl 3-allylpiperidine-1,3-dicarboxylate (9.1 g, 32.114 mmol) and 2,6-lutidine (6.88 g, 64.227 mmol) in dioxane (180 mL) and H₂O (180 mL) at 0° C. was added K₂OsO₄·2H₂O (591.61 mg, 1.606 mmol). The resulting mixture was stirred for 15 min at room temperature and was then cooled to at 0° C. and NaIO₄ (27.47 g, 128.455 mmol) was added in portions. The resulting mixture was stirred for 3 h at room temperature. And thee reaction was then quenched with sat. aq. Na₂S₂O₃ at 0° C. The resulting mixture was extracted with EtOAc (2×500 mL), and thee combined organic layers were washed with 1M HCl (2×200 mL), brine (2×200 mL), dried over Na₂SO₄, filtered, and concentrated under reduced pressure to afford the desired product (7.5 g, 81.9% yield). LCMS (ESI) m/z: [M+H] calcd for C₁₄H₂₃NO₅: 286.16; found 286.1.

### Step 3: Synthesis of 1-(tert-butyl) 3-methyl 3-(2-(((R)-1-(benzyloxy)-3-methyl-1-oxobutan-2-yl)amino)ethyl)piperidine-1,3-dicarboxylate

To a solution of 1-(tert-butyl) 3-methyl 3-(2-oxoethyl)piperidine-1,3-dicarboxylate (9.0 g, 31.541 mmol) and benzyl (2S)-2-amino-3-methylbutanoate (7.19 g, 34.695 mmol) in MeOH (90 mL) at 0° C. was added ZnCl₂ (4.73 g, 34.695 mmol) and NaBH₃CN (3.96 g, 63.083 mmol). The resulting mixture was stirred overnight at room temperature. Desired product could be detected by LCMS, and it was concentrated under reduced pressure and extracted with EtOAc (1200 mL). The organic layer was washed with brine (3×150 mL), dried over Na₂SO₄, filtered, and concentrated under reduced pressure. Purification by reverse phase chromatography afforded the desired product (9.9 g, 65.9% yield). LCMS (ESI) m/z: [M+H] calcd for C₂₆H₄₀N₂O₆: 477.29; found 477.2.

### Step 4: Synthesis of tert-butyl (S)-2-((S)-1-(benzyloxy)-3-methyl-1-oxobutan-2-yl)-1-oxo-2,7-diazaspiro[4.5]decane-7-carboxylate and tert-butyl (R)-2-((S)-1-(benzyloxy)-3-methyl-1-oxobutan-2-yl)-1-oxo-2,7-diazaspiro[4.5]decane-7-carboxylate

To a solution of 1-(tert-butyl) 3-methyl 3-(2-(((R)-1-(benzyloxy)-3-methyl-1-oxobutan-2-yl)amino)ethyl)piperidine-1,3-dicarboxylate (9.9 g, 20.772 mmol) and DIPEA (26.84 g, 207.715 mmol) in toluene (100 mL) was added DMAP (5.07 g, 41.543 mmol). The resulting mixture was stirred at 80° C. for 50 h. The resulting mixture was concentrated under reduced pressure and the residue was taken up in EtOAc (1000 mL). The organic layer was washed with brine (3×150 mL), dried over Na₂SO₄, filtered, and concentrated under reduced pressure. The crude product was purified by CHIRAL-HPLC (50% EtOH/Hex) to afford tert-butyl (S)-2-((S)-1-(benzyloxy)-3-methyl-1-oxobutan-2-yl)-1-oxo-2,7-diazaspiro[4.5]decane-7-carboxylate (1.75 g) and tert-butyl (R)-2-((S)-1-(benzyloxy)-3-methyl-1-oxobutan-2-yl)-1-oxo-2,7-diazaspiro[4.5]decane-7-carboxylate (1.98 g). LCMS (ESI) m/z: [M+H] calcd for C₂₅H₃₆N₂O₅: 445.26; found 445.2.

### Intermediates A-91 and A-92. Synthesis of (S)-2-((S)-7-(ter-butoxycarbonyl)-4-oxo-1-oxa-3,7-diazaspiro[4.4]nonan-3-yl)-3-methylbutanoic acid and (S)-2-((R)-7-(tert-butoxycarbonyl)-4-oxo-1-oxa-3,7-diazaspiro[4.4]nonan-3-yl)-3-methylbutanoic acid

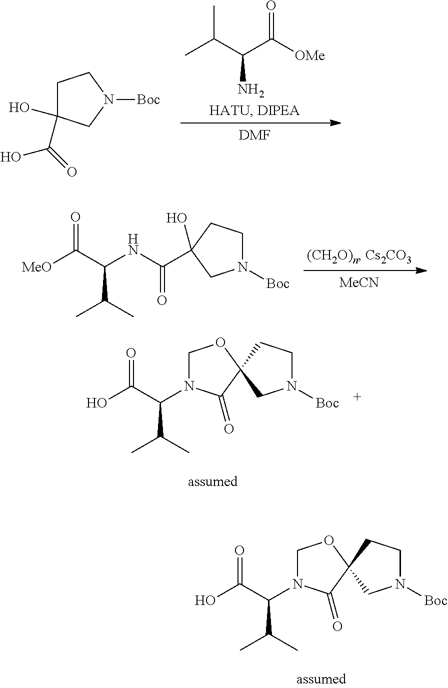

### Step 1: Synthesis of tert-butyl 3-hydroxy-3-(((S)-1-methoxy-3-methyl-1-oxobutan-2-yl)carbamoyl)pyrrolidine-1-carboxylate

To a solution of 1-(tert-butoxycarbonyl)-3-hydroxypyrrolidine-3-carboxylic acid (800 mg, 3.46 mmol) and DIPEA (3.01 mL, 17.3 mmol) in DMF (10 mL) at 0° C. was added methyl L-valinate (681 mg, 5.19 mmol) and HATU (1.71 g, 4.497 mmol). The resulting mixture was warmed to room temperature and stirred for 2 h then diluted with H₂O (20 mL) and extracted into EtOAc (3×20 mL). The combined organic layers were washed with brine (2×20 mL), dried over Na₂SO₄, filtered, and concentrated under reduced pressure. Purification by reverse phase chromatography (30→55% MeCN/H₂O, 0.1% NH₄HCO₃) afforded the desired product (1 g, 76% yield). LCMS (ESI) m/z: [M+Na] calcd for C₁₆H₂₈N₂O₆: 367.18; found 366.9.

### Step 2: Synthesis of (S)-2-((S)-7-(tert-butoxycarbonyl)-4-oxo-1-oxa-3,7-diazaspiro[4.4]nonan-3-yl)-3-methylbutanoic acid and (S)-2-((R)-7-(tert-butoxycarbonyl)-4-oxo-1-oxa-3,7-diazaspiro[4.4]nonan-3-yl)-3-methylbutanoic acid

To a solution of tert-butyl 3-hydroxy-3-(((S)-1-methoxy-3-methyl-1-oxobutan-2-yl)carbamoyl)pyrrolidine-1-carboxylate (1.0 g, 2.90 mmol) and Cs₂CO₃ (1.89 g, 5.81 mmol) in MeCN (15 mL) at 0° C. was added paraformaldehyde (436 mg, 14.5 mmol). The resulting mixture was heated to 80° C. and stirred overnight. Purification by reverse phase chromatography (10→40% MeCN/H₂O, 0.1% NH₄HCO₃) afforded a mixture of the desired products. The diastereomers were separated by prep-SFC (30% EtOH/hexanes, 0.3% TFA) to afford (S)-2-((S)-7-(tert-butoxycarbonyl)-4-oxo-1-oxa-3,7-diazaspiro[4.4]nonan-3-yl)-3-methylbutanoic acid (250 mg, 24% yield) and (S)-2-((R)-7-(tert-butoxycarbonyl)-4-oxo-1-oxa-3,7-diazaspiro[4.4]nonan-3-yl)-3-methylbutanoic acid (200 mg, 19% yield). LCMS (ESI) m/z: [M+Na] calcd for C₁₆H₂₆N₂O₆: 365.17; found 365.0.

### Intermediate A-93. Synthesis of (S)-1-((3-methyloxetan-3-yl)methyl)aziridine-2-carboxylic acid

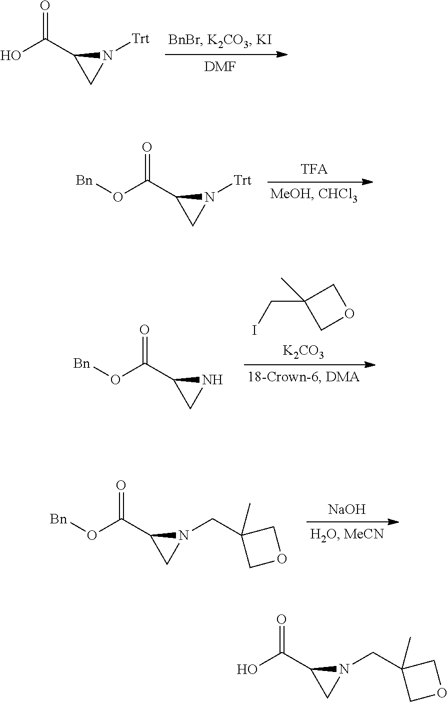

### Step 1: Synthesis of benzyl (S)-1-tritylaziridine-2-carboxylate

To a mixture of (S)-1-tritylaziridine-2-carboxylic acid (3.0 g, 9.11 mmol) and benzyl bromide (2.16 mL, 18.22 mmol) in DMF (30 mL) was added K₂CO₃ (2.25 g, 18.22 mmol) and KI (76 mg, 455 μmol). The reaction mixture was heated to 50° C. and stirred for 30 min then was cooled to room temperature and diluted with H₂O (30 mL) and EtOAc (30 mL). The aqueous layer was extracted with EtOAc (3×40 mL), and the combined organic layers were washed with brine (5×70 mL), dried over Na₂SO₄, filtered, and concentrated under reduced pressure to afford the desired product (4.7 g, crude).

### Step 2: Synthesis of benzyl (S)-aziridine-2-carboxylate

To a mixture of benzyl (S)-1-tritylaziridine-2-carboxylate (3.4 g, 8.10 mmol) in MeOH (17.5 mL) and CHCl₃ (17.5 mL) at 0° C. was added TFA (9.0 mL, 122 mmol). The reaction mixture was stirred for 30 min then was poured into sat. aq. NaHCO₃ (50 mL), extracted into DCM (4×35 mL), dried over Na₂SO₄, filtered, and concentrated under reduced pressure. Purification by silica gel column chromatography (6→100% EtOAc/pet. ether) afforded the desired product (445 mg, 31% yield).

### Step 3: Synthesis of benzyl (S)-1-((3-methyloxetan-3-yl)methyl)aziridine-2-carboxylate

To a mixture of benzyl (S)-aziridine-2-carboxylate (440 mg, 2.48 mmol) and 3-(iodomethyl)-3-methyloxetane (2.11 g, 9.93 mmol) in DMA (5 mL) was added K₂CO₃ (1.72 g, 12.42 mmol) and 18-crown-6 (32.8 mg, 124 μmol). The reaction mixture was heated to 80° C. and stirred for 12 h, and was then was diluted with H₂O (25 mL) and EtOAc (25 mL). The aqueous layer was extracted with EtOAc (3×20 mL), and the combined organic layers were washed with brine (5×45 mL), dried over Na₂SO₄, filtered, and concentrated under reduced pressure. Purification by prep-TLC (50% EtOAc/pet. ether) afforded the desired product (367 mg, 57% yield). LCMS (ESI) m/z: [M+H] calcd for C₁₅H₁₉NO₃: 262.14; found 262.0.

### Step 4: Synthesis of (S)-1-((3-methyloxetan-3-yl)methyl)aziridine-2-carboxylic acid

To a mixture of benzyl (S)-1-((3-methyloxetan-3-yl)methyl)aziridine-2-carboxylate (100 mg, 383 μmol) in MeCN (500 μL) and H₂O (500 μL) at 0° C. was added NaOH (23 mg, 574 μmol). The reaction mixture was stirred at 0° C. for 1 h then was concentrated under reduced pressure to afford the desired product (100 mg, crude). LCMS (ESI) m/z: [M+H] calcd for C₈H₁₃NO₃: 172.10; found 172.0.

### Intermediate A-94. Synthesis of (2R,3R)-1-(tert-butylsulfinyl)-3-ethylaziridine-2-carboxylic acid

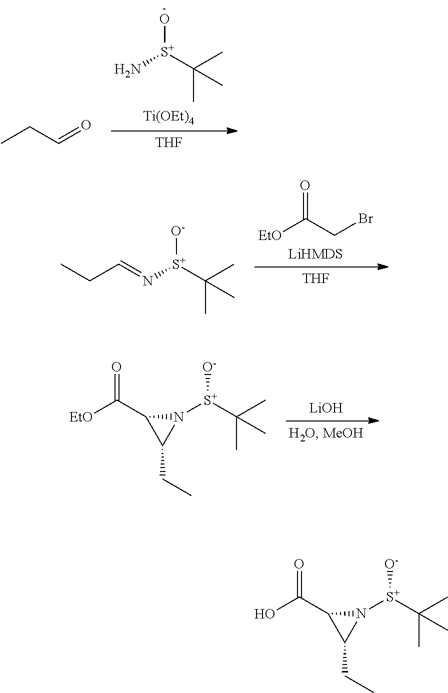

### Step 1: Synthesis of (R,E)-2-methyl-N-propylidenepropane-2-sulfinamide

To a solution of propionaldehyde (6.27 mL, 86.1 mmol) in THF (200 mL) was added (R)-2-methylpropane-2-sulfinamide (10.4 g, 86.1 mmol) and titanium ethoxide (51 mL, 170 mmol). The reaction mixture was heated to 70° C. for 3 h then cooled to room temperature and quenched with H₂O (50 mL), filtered, and extracted into EtOAc (3×30 mL). The combined organic layers were washed with brine (30 mL), dried over Na₂SO₄, filtered, and concentrated under reduced pressure. Purification by silica gel column chromatography (9→17% EtOAc/pet. ether) afforded the desired product (4.0 g, 29% yield).

### Step 2: Synthesis of ethyl (2R,3R)-1-(tert-butylsulfinyl)-3-ethylaziridine-2-carboxylate

To a solution of ethyl 2-bromoacetate (2.74 mL, 24.8 mmol) in THF (40 mL) at −78° C. was added LiHMDS (24.80 mL, 1 M in THF). After 30 min (R,E)-2-methyl-N-propylidenepropane-2-sulfinamide (2.0 g, 12.4 mmol) in THF (20 mL) was added to the reaction mixture. The mixture was stirred for 1 h then warmed to room temperature, quenched with H₂O (50 mL), and extracted into EtOAc (3×50 mL). The combined organic layers were washed with brine (2×50 mL), dried over Na₂SO₄, filtered, and concentrated under reduced pressure. Purification by silica gel column chromatography (17→25% EtOAc/pet. ether) afforded product (1.34 g, 44% yield). LCMS (ESI) m/z: [M+H] calcd for C₁₁H₂₁NO₃S: 248.13; found 248.1.

### Step 3: Synthesis of (2R,3R)-1-(tert-butylsulfinyl)-3-ethylaziridine-2-carboxylic acid

To a solution of ethyl (2R,3R)-1-(tert-butylsulfinyl)-3-ethylaziridine-2-carboxylate (600 mg, 2.4 mmol) in MeOH (3 mL) and H₂O (3 mL) was added LiOH (70 mg, 2.9 mmol). The resulting mixture was stirred for 16 h then diluted with H₂O (20 mL) and washed with DCM (3×10 mL). Lyophilization of the aqueous layer afforded product (600 mg, crude). LCMS (ESI) m/z: [M+H] calcd for C₉H₁₇NO₃S: 220.10; found 220.3.

### Intermediate A-95. Synthesis of (2S,3S)-1-(tert-butylsulfinyl)-3-ethylaziridine-2-carboxylic acid

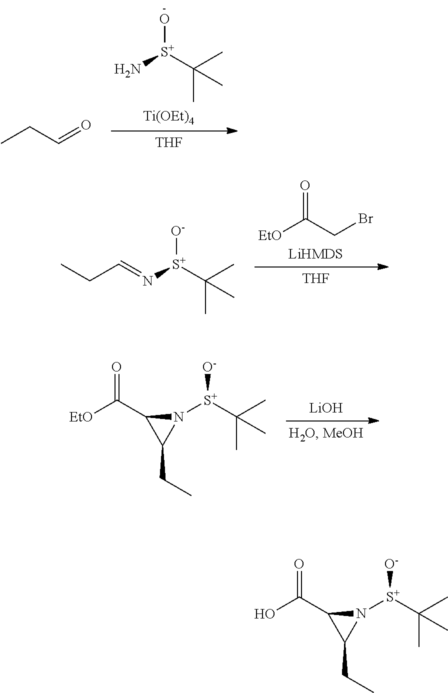

### Step 1: Synthesis of (S,E)-2-methyl-N-propylidenepropane-2-sulfinamide

To a solution of propionaldehyde (6.27 mL, 86.1 mmol) in THF (50 mL) was added (S)-2-methylpropane-2-sulfinamide (10.4 g, 86.1 mmol) and titanium ethoxide (51 mL, 170 mmol). The reaction mixture was heated to 70° C. for 3 h then cooled to room temperature and quenched with H₂O (30 mL), filtered, and extracted into DCM (3×100 mL). The combined organic layers were washed with brine (10 mL), dried over Na₂SO₄, filtered, and concentrated under reduced pressure. Purification by silica gel column chromatography (25% EtOAc/pet. ether) afforded product (4.6 g, 33% yield).

### Step 2: Synthesis of ethyl (2S,3S)-1-(tert-butylsulfinyl)-3-ethylaziridine-2-carboxylate

To a solution of ethyl 2-bromoacetate (2.74 mL, 24.8 mmol) in THF (40 mL) at −78° C. was added LiHMDS (24.80 mL, 1M in THF). After 30 min (S,E)-2-methyl-N-propylidenepropane-2-sulfinamide (2.0 g, 12.4 mmol) in THF (20 mL) was added to the reaction mixture. The mixture was stirred for 1 h then warmed to room temperature, quenched with H₂O (20 mL), and extracted into EtOAc (3×20 mL). The combined organic layers were washed with brine (2×25 mL), dried over Na₂SO₄, filtered, and concentrated under reduced pressure. Purification by reverse phase chromatography (31→51% MeCN/H₂O, 10 mM NH₄HCO₃) afforded product (600 mg, 20% yield). LCMS (ESI) m/z: [M+H] calcd for C₁₁H₂₁NO₃S: 248.13; found 248.1.

### Step 3: Synthesis of (2S,3S)-1-(tert-butylsulfinyl)-3-ethylaziridine-2-carboxylic acid

To a solution of ethyl (2S,3S)-1-(tert-butylsulfinyl)-3-ethylaziridine-2-carboxylate (600 mg, 2.4 mmol) in MeOH (300 μL) and H₂O (300 μL) was added LiOH (87 mg, 3.6 mmol). The resulting mixture was stirred for 12 h then diluted with H₂O (20 mL) and washed with DCM (3×10 mL). Lyophilization of the aqueous layer afforded product (600 mg, crude). LCMS (ESI) m/z: [M+H] calcd for C₉H₁₇NO₃S: 220.10; found 220.2.

### Intermediate A-96. Synthesis of (2R,3R)-3-isopropyl-1-tritylaziridine-2-carboxylic acid

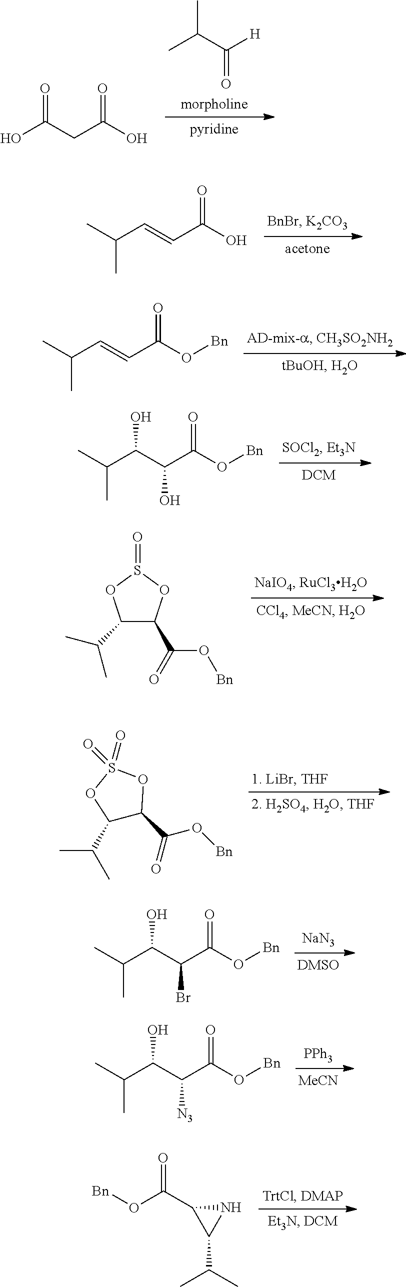

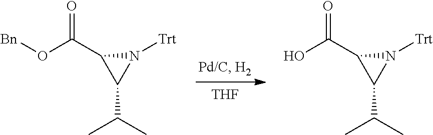

### Step 1: Synthesis of (E)-4-methylpent-2-enoic acid

Two batches of a solution of malonic acid (25.0 mL, 240 mmol), isobutyraldehyde (34.7 mL, 380 mmol) and morpholine (380 μL, 4.32 mmol) in pyridine (75 mL) were stirred for 24 h then were heated to 115° C. and stirred for 12 h. The combined reaction mixtures were poured into H₂SO₄ (1M, 800 mL) and extracted into EtOAc (3×300 mL). The combined organic layers were washed with brine (300 mL), dried over Na₂SO₄, filtered, and concentrated under reduced pressure. The residue was dissolved in NaOH (1 M, 500 mL), washed with EtOAc (2×200 mL), acidified to pH=4−2 with HCl (4M), and extracted into EtOAc (3×300 mL). The combined organic layers were washed with brine (300 mL), dried over Na₂SO₄, filtered, and concentrated under reduced pressure which afforded product (54 g, 98% yield).

### Step 2: Synthesis of benzyl (E)-4-methylpent-2-enoate

To two batches of a solution of (E)-4-methylpent-2-enoic acid (6.25 mL, 52.6 mmol) in acetone (90 mL) was added K₂CO₃ (13.8 g, 100 mmol) and the mixtures were stirred for 30 min. Then a solution of benzyl bromide (6.31 mL, 53.1 mmol) in acetone (10 mL) was added and the mixtures were heated to 75° C. for 5 h. The reaction mixtures were cooled to room temperature and concentrated under reduced pressure. The residue was dissolved in EtOAc (200 mL) and H₂O (200 mL) then extracted into EtOAc (2×200 mL). The combined organic layers were washed with brine (300 mL), dried over Na₂SO₄, filtered, and concentrated under reduced pressure. Purification by silica gel chromatography (0→10% EtOAc/pet. ether) afforded product (9.0 g, 42% yield).

### Step 3: Synthesis of benzyl (2R,3S)-2,3-dihydroxy-4-methylpentanoate

To a solution of AD-mix-α (61.7 g) and methanesulfonamide (4.19 g, 44.1 mmol) in tert-BuOH (225 mL) and H₂O (225 mL) was added benzyl (E)-4-methylpent-2-enoate (9 g, 44.1 mmol). The mixture was stirred at room temperature for 12 h then Na₂SO₃ (67.5 g) was added and stirred for 30 min. The reaction mixture was diluted with EtOAc (300 mL) and H₂O (300 mL) and extracted into EtOAc (3×300 mL), washed with brine (300 mL), dried over Na₂SO₄, filtered, and concentrated under reduced pressure. Purification by silica gel chromatography (0→25% EtOAc/pet. ether) afforded product (8.3 g, 79% yield). LCMS (ESI) m/z: [M+Na] calcd for C₁₃H₁₈O₄: 261.11; found 261.0.

### Step 4: Synthesis of benzyl (4R,5S)-5-isopropyl-1,3,2-dioxathiolane-4-carboxylate 2-oxide

To a solution of benzyl (2R,3S)-2,3-dihydroxy-4-methylpentanoate (10 g, 42.0 mmol) in DCM (100 mL) at 0° C. was added Et₃N (17.5 mL, 126 mmol) and SOCl₂ (4.26 mL, 58.8 mmol). The reaction mixture was stirred 30 min then was diluted with DCM (30 mL) and H₂O (100 mL), extracted into DCM (3×50 mL), washed with brine (100 mL), dried over Na₂SO₄, filtered, and concentrated under reduced pressure which afforded product (11.0 g, 92% yield).

### Step 5: Synthesis of benzyl (4R,5S)-5-isopropyl-1,3,2-dioxathiolane-4-carboxylate 2,2-dioxide

To a solution of benzyl (4R,5S)-5-isopropyl-1,3,2-dioxathiolane-4-carboxylate 2-oxide (11 g, 38.7 mmol) in H₂O (250 mL), MeCN (125 mL), and CCl₄ (125 mL) was added NaIO₄ (3.22 mL, 58.0 mmol) and RuCl₃·H₂O (872 mg, 3.87 mmol). The mixture was stirred at room temperature for 1 h then was diluted with EtOAc (200 mL) and H₂O (50 mL), filtered, and the filtrate was extracted into EtOAc (3×200 mL). The combined organic layers were washed sequentially with brine (200 mL) and sat. aq. Na₂CO₃ (300 mL), dried over Na₂SO₄, filtered, and concentrated under reduced pressure. Purification by silica gel chromatography (0→17% EtOAc/pet. ether) afforded product (11 g, 95% yield).

### Step 6: Synthesis of benzyl (2S,3S)-2-bromo-3-hydroxy-4-methylpentanoate

To a solution of benzyl (4R,5S)-5-isopropyl-1,3,2-dioxathiolane-4-carboxylate 2,2-dioxide (11 g, 36.6 mmol) in THF (520 mL) was added LiBr (3.49 mL, 139 mmol). The reaction mixture was stirred at room temperature for 5 h and then concentrated under reduced pressure. The residue was diluted in THF (130 mL) and H₂O (65 mL), cooled to 0° C., then H₂SO₄ solution (20% aq., 1.3 L) was added and the mixture was warmed to room temperature and stirred for 24 h. The mixture was diluted with EtOAc (1.0 L), extracted into EtOAc (2×300 mL), washed sequentially with Na₂CO₃ (sat. aq., 300 mL) and brine (300 mL), then was concentrated under reduced pressure. Purification by silica gel chromatography (0→17% EtOAc/pet. ether) afforded product (10 g, 81% yield).

### Step 7: Synthesis of benzyl (2R,3S)-2-azido-3-hydroxy-4-methylpentanoate

To a solution of benzyl (2S,3S)-2-bromo-3-hydroxy-4-methylpentanoate (10 g, 33.2 mmol) in DMSO (100 mL) was added NaN₃ (4.32 g, 66.4 mmol). The reaction mixture was stirred at room temperature for 12 h then was diluted with EtOAc (300 mL) and H₂O (200 mL). The aqueous phase was extracted into EtOAc (2×200 mL), washed with brine (200 mL), dried over Na₂SO₄, filtered, and concentrated under reduced pressure. Purification by silica gel chromatography (0→17% EtOAc/pet. ether) afforded product (7.5 g, 79% yield).

### Step 8: Synthesis of benzyl (2R,3R)-3-isopropylaziridine-2-carboxylate

To a solution of benzyl (2R,3S)-2-azido-3-hydroxy-4-methylpentanoate (7.5 g, 28.5 mmol) in MeCN (150 mL) was added PPh₃ (7.70 g, 29.3 mmol). The reaction mixture was stirred at room temperature for 1 h and then heated to 70° C. and stirred for 4 h. The reaction mixture was concentrated under reduced pressure and purification by silica gel chromatography (0→17% EtOAc/pet. ether) afforded product (4.5 g, 66% yield). LCMS (ESI) m/z: [M+H] calcd for C₁₃H₁₇NO₂: 220.13; found 220.0.

### Step 9: Synthesis of benzyl (2R,3R)-3-isopropyl-1-tritylaziridine-2-carboxylate

To a solution of benzyl (2R,3R)-3-isopropylaziridine-2-carboxylate (2 g, 9.12 mmol) in DCM (30 mL) at 0° C. was added Et₃N (3.81 mL, 27.4 mmol) and trityl chloride (3.05 g, 10.9 mmol) followed by DMAP (111 mg, 912 μmol). The reaction mixture was stirred at 0° C. for 1 h and then was diluted with DCM (50 mL) and H₂O (50 mL) then extracted into DCM (2×30 mL). The combined organic layers were washed with brine (50 mL), dried over Na₂SO₄, filtered, and concentrated under reduced pressure. Purification by silica gel chromatography (0→25% DCM/pet. ether) afforded product (3.1 g, 72% yield).

### Step 10: Synthesis of (2R,3R)-3-isopropyl-1-tritylaziridine-2-carboxylic acid

Two solutions of benzyl (2R,3R)-3-isopropyl-1-tritylaziridine-2-carboxylate (200 mg, 430 μmol) and Pd/C (100 mg) in THF (4 mL) were stirred for 1 h at room temperature under H₂ atmosphere. The reaction mixtures were combined, filtered, and concentrated under reduced pressure. Purification by silica gel chromatography (0→50% EtOAc/pet. ether) afforded product (160 mg, 51% yield).

### Intermediate A-97. Synthesis of (2S,3S)-1-benzyl-3-isopropylaziridine-2-carboxylic acid

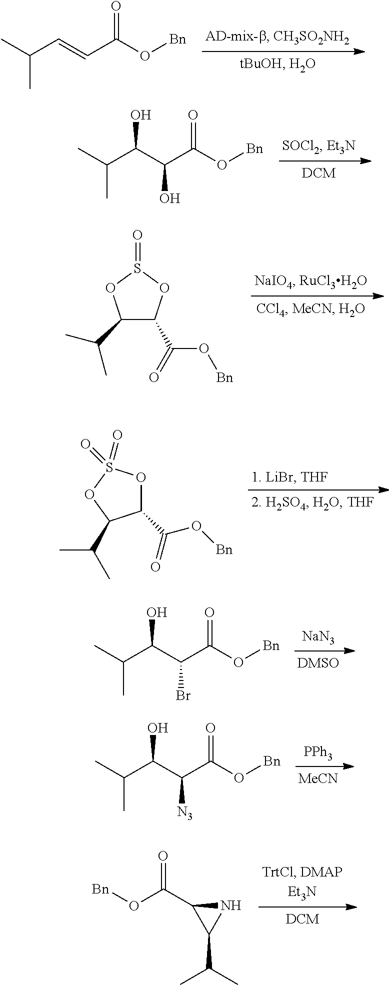

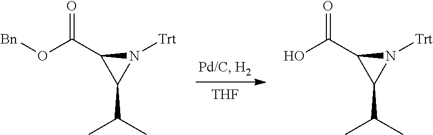

### Step 1: Synthesis of benzyl (2S,3R)-2,3-dihydroxy-4-methylpentanoate

To a solution of AD-mix-β (61.7 g) and methanesulfonamide (4.19 g, 44.1 mmol) in tert-BuOH (225 mL) and H₂O (225 mL) was added benzyl (E)-4-methylpent-2-enoate (9 g, 44.1 mmol). The mixture was stirred at room temperature for 12 h then Na₂SO₃ (67.5 g) was added and stirred for 30 min. The reaction mixture was diluted with EtOAc (300 mL) and H₂O (300 mL) and extracted into EtOAc (3×300 mL), washed with brine (300 mL), dried over Na₂SO₄, filtered, and concentrated under reduced pressure. Purification by silica gel chromatography (0→25% EtOAc/pet. ether) afforded product (8.8 g, 84% yield). LCMS (ESI) m/z: [M+Na] calcd for C₁₃H₁₈O₄: 261.11; found 261.0.

### Step 2: Synthesis of benzyl (4S,5R)-5-isopropyl-1,3,2-dioxathiolane-4-carboxylate 2-oxide

To a solution of benzyl (2S,3R)-2,3-dihydroxy-4-methylpentanoate (11.6 g, 48.7 mmol) in DCM (116 mL) at 0° C. was added Et₃N (20.3 mL, 146 mmol) and SOCl₂ (4.94 mL, 68.2 mmol). The reaction mixture was stirred 30 min then was diluted with DCM (100 mL) and H₂O (100 mL), extracted into DCM (3×100 mL), washed with brine (200 mL), dried over Na₂SO₄, filtered, and concentrated under reduced pressure which afforded product (13.0 g, 94% yield).

### Step 3: Synthesis of benzyl (4S,5R)-5-isopropyl-1,3,2-dioxathiolane-4-carboxylate 2,2-dioxide

To a solution of benzyl (4S,5R)-5-isopropyl-1,3,2-dioxathiolane-4-carboxylate 2-oxide (13 g, 45.7 mmol) in H₂O (290 mL), MeCN (145 mL), and CCl₄ (145 mL) was added NaIO₄ (3.80 mL, 68.6 mmol) and RuCl₃·H₂O (1.03 g, 4.57 mmol). The mixture was stirred at room temperature for 1 h then was diluted with DCM (500 mL) and H₂O (300 mL), filtered, and the filtrate was extracted into DCM (3×200 mL). The combined organic layers were washed sequentially with brine (500 mL) and sat. aq. Na₂CO₃ (300 mL), dried over Na₂SO₄, filtered, and concentrated under reduced pressure. Purification by silica gel chromatography (0→17% EtOAc/pet. ether) afforded product (11.5 g, 80% yield).

### Step 4: Synthesis of benzyl (2R,3R)-2-bromo-3-hydroxy-4-methylpentanoate

To a solution of benzyl (4S,5R)-5-isopropyl-1,3,2-dioxathiolane-4-carboxylate 2,2-dioxide (11.5 g, 38.3 mmol) in THF (520 mL) was added LiBr (3.65 mL, 146 mmol). The reaction mixture was stirred at room temperature for 5 h and then concentrated under reduced pressure. The residue was diluted in THF (130 mL) and H₂O (65 mL), cooled to 0° C., then H₂SO₄ solution (20% aq., 1.3 L) was added and the mixture was warmed to room temperature and stirred for 24 h. The mixture was diluted with EtOAc (1.0 L), washed with Na₂CO₃ (sat. aq., 300 mL), then was concentrated under reduced pressure. Purification by silica gel chromatography (0→17% EtOAc/pet. ether) afforded product (10 g, 83% yield).

### Step 5: Synthesis of benzyl (2S,3R)-2-azido-3-hydroxy-4-methylpentanoate

To a solution of benzyl (2R,3R)-2-bromo-3-hydroxy-4-methylpentanoate (10 g, 33.2 mmol) in DMSO (100 mL) was added NaN₃ (4.33 g, 66.6 mmol). The reaction mixture was stirred at room temperature for 12 h then was diluted with EtOAc (300 mL) and H₂O (200 mL). The mixture was extracted into EtOAc (2×200 mL), dried over Na₂SO₄, filtered, and concentrated under reduced pressure. Purification by silica gel chromatography (0→17% EtOAc/pet. ether) afforded product (7.5 g, 76% yield).

### Step 6: Synthesis of benzyl (2S,3S)-3-isopropylaziridine-2-carboxylate

To a solution of benzyl (2S,3R)-2-azido-3-hydroxy-4-methylpentanoate (7.5 g, 28.5 mmol) in MeCN (150 mL) was added PPh₃ (7.70 g, 29.3 mmol). The reaction mixture was stirred at room temperature for 1 h and then heated to 70° C. and stirred for 3 h. The reaction mixture was concentrated under reduced pressure and purification by silica gel chromatography (0→17% EtOAc/pet. ether) afforded product (4.5 g, 64% yield). LCMS (ESI) m/z: [M+H] calcd for C₁₃H₁₇NO₂: 220.13; found 220.1.

### Step 7: Synthesis of benzyl (2S,3S)-1-benzyl-3-isopropylaziridine-2-carboxylate

To a solution of benzyl (2S,3S)-3-isopropylaziridine-2-carboxylate (1 g, 4.56 mmol) in MeCN (10 mL) was added K₂CO₃ (3.15 g, 22.8 mmol) and benzyl bromide (812 μL, 6.84 mmol). The reaction mixture was stirred at room temperature for 6 h then was diluted with EtOAc (30 mL) and H₂O (30 mL), extracted into EtOAc (2×30 mL), washed with brine (50 mL), dried over Na₂SO₄, filtered, and concentrated under reduced pressure. Purification by silica gel chromatography (0→17% EtOAc/pet. ether) afforded product (1.3 g, 89% yield). LCMS (ESI) m/z: [M+H] calcd for C₂₀H₂₃NO₂: 310.18; found 310.1.

### Step 8: Synthesis of (2S,3S)-1-benzyl-3-isopropylaziridine-2-carboxylic acid

To a solution of benzyl (2S,3S)-1-benzyl-3-isopropylaziridine-2-carboxylate (600 mg, 1.94 mmol) in THF (6 mL), MeCN (3 mL), and H₂O (6 mL) at 0° C. was added LiOH·H₂O (163 mg, 3.88 mmol). The reaction mixture was stirred at room temperature for 1 h and was adjusted to pH=7-8 with HCl (0.5M). Lyophilization afforded product (750 mg, crude). LCMS (ESI) m/z: [M+H] calcd for C₁₃H₁₇NO₂: 220.13; found 220.1.

### Intermediate A-98, A-99, A-100, and A-101. Synthesis of ethyl (2R,3R)-1-benzhydryl-3-(oxetan-3-yl)aziridine-2-carboxylate, ethyl (2S,3S)-1-benzhydryl-3-(oxetan-3-yl)aziridine-2-carboxylate, ethyl (2R,3S)-1-benzhydryl-3-(oxetan-3-yl)aziridine-2-carboxylate, and ethyl (2S,3R)-1-benzhydryl-3-(oxetan-3-yl)aziridine-2-carboxylate

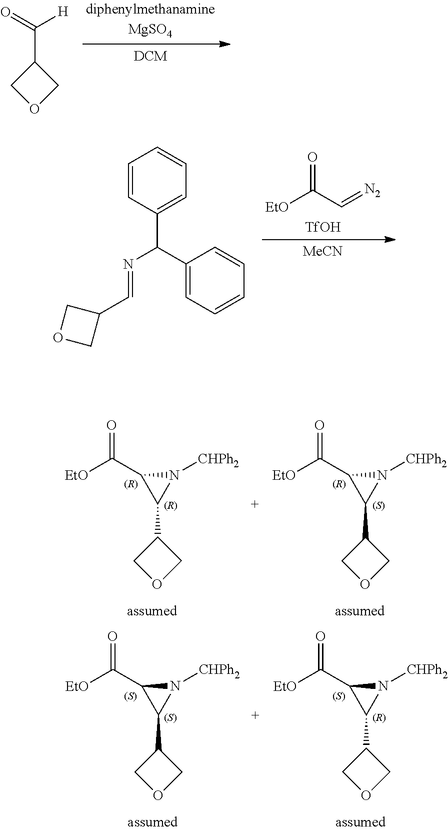

### Step 1: Synthesis of N-benzhydryl-1-(oxetan-3-yl)methanimine

To a solution oxetane-3-carbaldehyde (5.0 g, 58 mmol) and MgSO₄ (6.99 g, 58.1 mmol) in DCM (120 mL) at 0° C. was added diphenylmethanamine (12.1 mL, 69.7 mmol). The mixture was stirred for 12 h at room temperature then filtered and concentrated under reduced pressure to afford the desired compound (14 g, 95.9% yield) which was used without further purification.

### Step 2: Synthesis of ethyl cis-1-benzhydryl-3-(oxetan-3-yl)aziridine-2-carboxylate and ethyl trans-1-benzhydryl-3-(oxetan-3-yl)aziridine-2-carboxylate

To a solution of N-benzhydryl-1-(oxetan-3-yl)methanimine (10 g, 39.79 mmol) in MeCN (150 mL) was added TfOH (878 mL, 9.95 mmol) and after 5 min ethyl diazoacetate (5.0 mL, 47.8 mmol) was added. The reaction mixture was stirred for 12 h at room temperature then cooled to 0° C. and quenched by the addition of saturated NaHCO₃ (300 mL). The aqueous layer was extracted with EtOAc (3×200 mL) and the combined organic layers were washed with brine, dried with Na₂SO₄, filtered, and concentrated under reduced pressure. Purification by reverse phase chromatography (50→65% MeCN/H₂O, 10 mM NH₄HCOS) afforded racemic ethyl cis-1-benzhydryl-3-(oxetan-3-yl)aziridine-2-carboxylate (1.1 g, 8.2% yield) and racemic ethyl trans-1-benzhydryl-3-(oxetan-3-yl)aziridine-2-carboxylate (780 mg, 5.8% yield) Step 3: Separation of racemic ethyl cis-1-benzhydryl-3-(oxetan-3-yl)aziridine-2-carboxylate: ethyl (2R,3R)-1-benzhydryl-3-(oxetan-3-yl)aziridine-2-carboxylate and ethyl (2S,3S)-1-benzhydryl-3-(oxetan-3-yl)aziridine-2-carboxylate Racemic ethyl cis-1-benzhydryl-3-(oxetan-3-yl)aziridine-2-carboxylate (800 mg, 2.37 mmol) was separated by chiral prep-SFC (25% MeOH/CO₂) to afford ethyl (2R,3R)-1-benzhydryl-3-(oxetan-3-yl) aziridine-2-carboxylate (320 mg, 40% yield) and ethyl (2S,3S)-1-benzhydryl-3-(oxetan-3-yl)aziridine-2-carboxylate (320 mg, 40% yield).

### Step 4: Separation of racemic ethyl trans-1-benzhydryl-3-(oxetan-3-yl)aziridine-2-carboxylate: ethyl (2R,3S)-1-benzhydryl-3-(oxetan-3-yl)aziridine-2-carboxylate and ethyl (2S,3R)-1-benzhydryl-3-(oxetan-3-yl)aziridine-2-carboxylate

Racemic ethyl trans1-benzhydryl-3-(oxetan-3-yl)aziridine-2-carboxylate (700 mg, 2.07 mmol) was separated by chiral prep-SFC (25% EtOH, 0.1% NH₄OH/CO₂) to afford ethyl (2R,3S)-1-benzhydryl-3-(oxetan-3-yl) aziridine-2-carboxylate (300 mg, 42% yield) and ethyl (2S,3R)-1-benzhydryl-3-(oxetan-3-yl)aziridine-2-carboxylate (320 mg, 43% yield).

### Intermediate A-102 and A-103. Synthesis of (2R,3R)-1-benzhydryl-3-(oxetan-3-yl)aziridine-2-carboxylic acid and (2S,3S)-1-benzhydryl-3-(oxetan-3-yl)aziridine-2-carboxylic acid

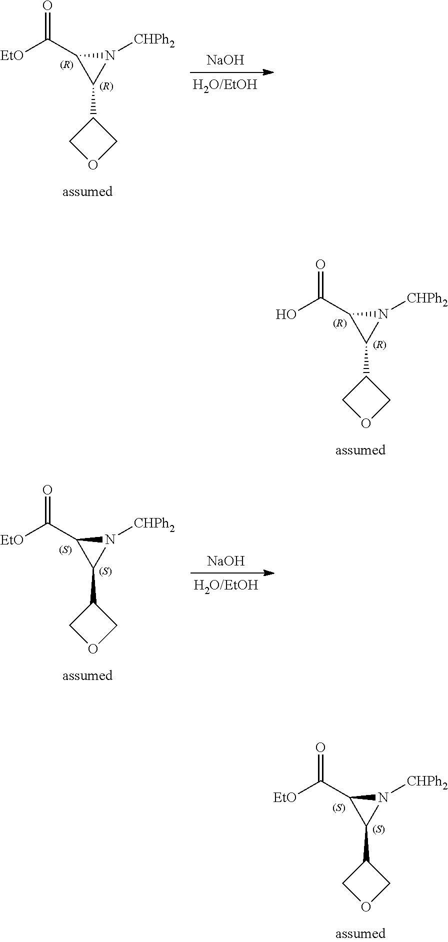

### Step 1: Synthesis of (2R,3R)-1-benzhydryl-3-(oxetan-3-yl)aziridine-2-carboxylic acid

To a solution of ethyl (2R,3R)-1-benzhydryl-3-(oxetan-3-yl)aziridine-2-carboxylate (156 mg, 463 mmol) in EtOH (3 mL) was added 2M NaOH (347 mL, 696 mmol). The reaction mixture was stirred for 3 h at room temperature and then concentrated under reduced pressure. The concentrate was acidified to pH 5 with 1M HCl and extracted with DCM (3×5 mL) and the combined organic layers were washed with brine, dried with Na₂SO₄, filtered and concentrated under reduced pressure to afford the desired compound (110 mg, 72.6% yield).

### Step 2: Synthesis of (2S,3S)-1-benzhydryl-3-(oxetan-3-yl)aziridine-2-carboxylic acid

To a solution of ethyl (2S,3S)-1-benzhydryl-3-(oxetan-3-yl)aziridine-2-carboxylate (150 mg, 444 mmol) in EtOH (5 mL) was added 2M NaOH (333 mL, 666 mmol). The reaction mixture was stirred for 3 h at room temperature and then acidified to pH 5 with 1M HCl. The aqueous layer extracted with DCM (3×10 mL) and the combined organic layers were washed with brine, dried with Na₂SO₄, filtered, and concentrated under reduced pressure to afford the desired compound (120 mg, 86.1% yield).

### Intermediate A-104 and A-105. Synthesis of sodium (2R,3S)-1-benzhydryl-3-(oxetan-3-yl)aziridine-2-carboxylate and sodium (2S,3R)-1-benzhydryl-3-(oxetan-3-yl)aziridine-2-carboxylate

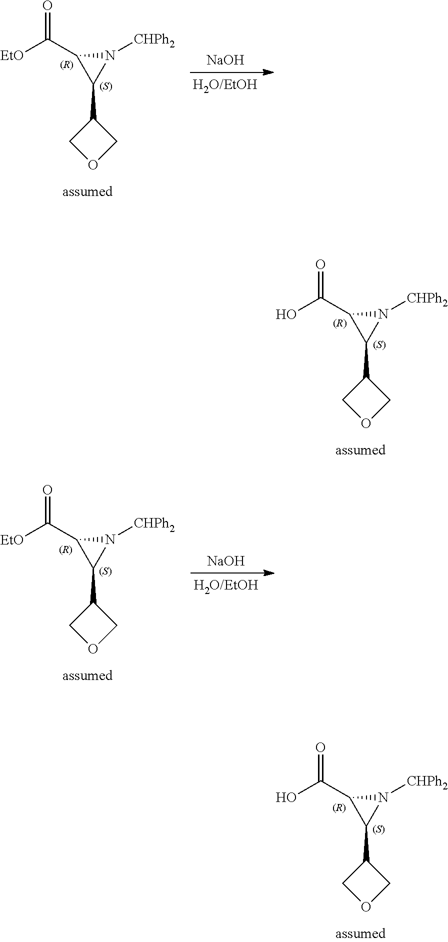

### Step 1: Synthesis of sodium (2R,3S)-1-benzhydryl-3-(oxetan-3-yl)aziridine-2-carboxylate

To a solution of ethyl (2R,3S)-1-benzhydryl-3-(oxetan-3-yl)aziridine-2-carboxylate (150 mg, 444 mmol) in EtOH (3 mL) was added 2M NaOH (333.42 mL, 666 mmol). The reaction mixture was stirred for 3 h at room temperature and then the pH was adjusted to pH 8 with 1M HCl. The resulting solution was lyophilized to afford the desired compound (165 mg, crude) which was used without further purification. LCMS (ESI) m/z: [M] calcd for C₁₉H₁₈NO₃: 308.13; found 308.0.

### Step 2: Synthesis of sodium (2S,3R)-1-benzhydryl-3-(oxetan-3-yl)aziridine-2-carboxylate

To a solution of ethyl (2S,3R)-1-benzhydryl-3-(oxetan-3-yl)aziridine-2-carboxylate (170 mg, 503 mmol) in EtOH (3 mL) was added 2M NaOH (378 mL, 754 mmol). The reaction mixture was stirred for 3 h at room temperature and then the pH was adjusted to pH 8 with 1M HCl. The resulting solution was lyophilized to afford the desired compound (230 mg, crude) which was used without further purification. LCMS (ESI) m/z: [M] calcd for C₁₉H₁₈NO₃: 308.13; found 308.0.

### Intermediate A-106. Synthesis of (R)-1-((benzyloxy)carbonyl)-2-methylaziridine-2-carboxylic acid

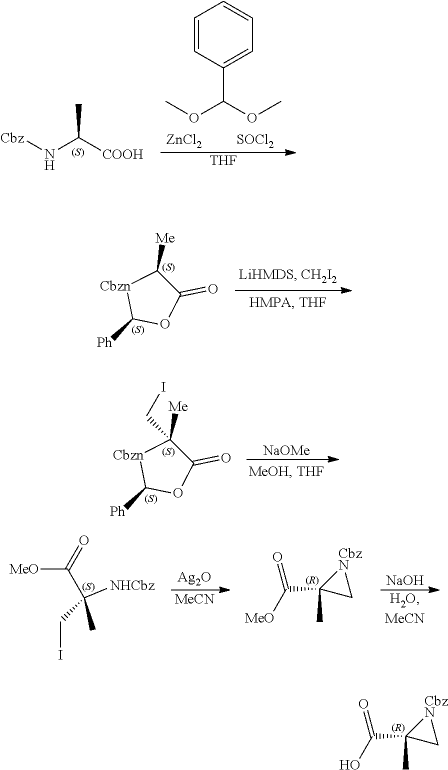

### Step 1: Synthesis of benzyl (2S,4S)-4-methyl-5-oxo-2-phenyloxazolidine-3-carboxylate

To a mixture of ((benzyloxy)carbonyl)-L-alanine (25 g, 111.99 mmol) and (dimethoxymethyl)benzene (71.38 mL, 115.35 mmol) in THF (180 mL) was added SOCl₂ (8.94 g, 123.19 mmol) in one portion at 0° C. The mixture was stirred for 10 min before ZnCl₂ (5.77 mL, 123.26 mmol) was added to the solution, then the mixture was stirred at 0° C. for 4 h. The reaction mixture was quenched by dropwise addition of cold H₂O and adjusted to pH=5 with sat. NaHCO₃, then extracted with EtOAc (2×100 mL). The organic phase was washed with a aq. sat. NaHCO₃ (30 mL) and brine (30 mL), dried over Na₂SO₄, and concentrated under reduced pressure. The residue was purified by silica gel column chromatography (1→10% EtOAc/pet. ether) to afford product (15 g, 43% yield).

### Step 2: Synthesis of benzyl (2S,4S)-4-(iodomethyl)-4-methyl-5-oxo-2-phenyloxazolidine-3-carboxylate

HMPA (5.22 mL, 29.74 mmol) and LHMDS (1 M, 6.62 mL) were mixed in THF (45 mL) under N₂ atmosphere at 20° C. This solution was cooled to −78° C. and a solution of benzyl (2S,4S)-4-methyl-5-oxo-2-phenyloxazolidine-3-carboxylate (2.0 g, 6.42 mmol) in THF (12 mL) was added dropwise with stirring. After stirring an additional 30 min, a solution of CH₂I₂ (1.55 mL, 19.27 mmol) in THF (6 mL) was added dropwise. The mixture was stirred at −78° C. for 90 min. The mixture was warmed to 0° C. and quenched with sat. aq. NH₄Cl (70 mL). The mixture was extracted with EtOAc (2×30 mL), and the combined organic layers was washed with sat. aq. NH₄Cl (20 mL), H₂O (2×20 mL), and brine (30 mL) dried over Na₂SO₄, filtered, and concentrated under reduced pressure. The resulting residue was purified by silica gel column chromatography (1→20% EtOAc/pet. ether) to afford product (1.2 g, 41.4% yield).

### Step 3: Synthesis of methyl (S)-2-(((benzyloxy)carbonyl)amino)-3-iodo-2-methylpropanoate

To a mixture of benzyl (2S,4S)-4-(iodomethyl)-4-methyl-5-oxo-2-phenyloxazolidine-3-carboxylate (1.2 g, 2.66 mmol) in THF (20 mL) was added a solution of NaOMe (957.69 mg, 5.32 mmol, 30% purity) in MeOH (9 mL) dropwise over 10 min at −40° C. under N₂. The mixture was stirred at −40° C. for 2 h, then warmed to −20° C. and stirred for 1 h. The reaction was quenched by addition of H₂O (20 mL), and the resulting mixture was extracted with EtOAc (3×20 mL). The combined organic layers were washed with brine (20 mL), dried over Na₂SO₄, filtered, and concentrated under reduced pressure. The residue was purified by silica gel column chromatography (1→20% EtOAc/pet. ether) to afford product (870 mg, 2.24 mmol, 84.4% yield).

### Step 4: Synthesis of 1-benzyl 2-methyl (R)-2-methylaziridine-1,2-dicarboxylate

To a mixture of methyl (S)-2-(((benzyloxy)carbonyl)amino)-3-iodo-2-methylpropanoate (0.87 g, 2.31 mmol) in MeCN (125 mL) was added Ag₂O (1.60 g, 6.92 mmol) in one portion at room temperature. The mixture was stirred at 90° C. for 30 min. The mixture was filtered and concentrated under reduced pressure to afford product (500 mg, 2.01 mmol, 86.9% yield).

### Step 5: Synthesis of 1-benzyl 2-methyl (R)-2-methylaziridine-1,2-dicarboxylate

To a mixture of 1-benzyl 2-methyl (R)-2-methylaziridine-1,2-dicarboxylate (250 mg, 1.0 mmol) in MeCN (2.5 mL) and H₂O (2.5 mL) was added NaOH (40.12 mg, 1.0 mmol) in one portion at 0° C. under N₂. The mixture was stirred at 0° C. for 30 min. The mixture was concentrated under reduced pressure to afford crude product (256 mg, crude). LCMS (ESI) m/z: [M+H] calcd for C₁₂H₁₂NO₄: 234.1; found 234.1.

### Intermediate A-107. Synthesis of (S)-1-((benzyloxy)carbonyl)-2-methylaziridine-2-carboxylic acid

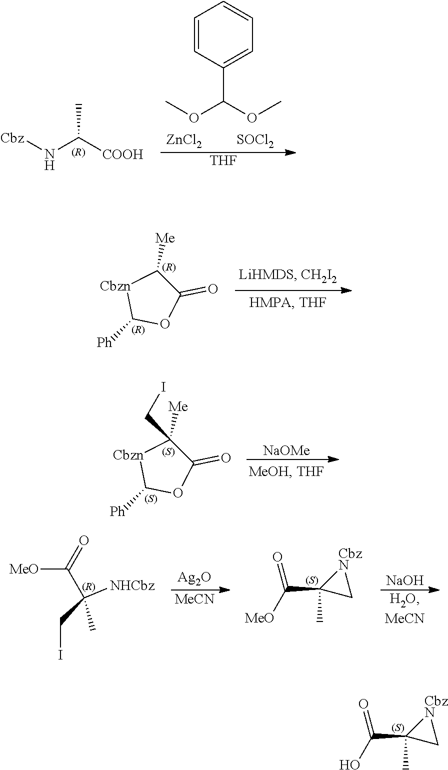

### Step 1: Synthesis of benzyl (2R,4R)-4-methyl-5-oxo-2-phenyloxazolidine-3-carboxylate

Five batches were completed in parallel. To a mixture of ((benzyloxy)carbonyl)-D-alanine (5 g, 22.40 mmol) and (dimethoxymethyl)benzene (3.71 mL, 24.64 mmol) in THF (35 mL) was added SOCl₂ (1.79 mL, 24.64 mmol) in one portion at 0° C. After the mixture was stirred for 10 min, ZnCl₂ (1.15 mL, 24.64 mmol) was added to the solution. Then the mixture was stirred at 0° C. for 4 h. The give batches were combined and concentrated under reduced pressure. The residue was purified by silica gel column chromatography (1→10% EtOAc/pet. ether) to afford product (20 g, 57.4% yield).

### Step 2: Synthesis of benzyl (2R,4R)-4-(iodomethyl)-4-methyl-5-oxo-2-phenyloxazolidine-3-carboxylate

Four batches were completed in parallel. THF (300 mL), HMPA (13.06 mL, 74.34 mmol) and LHMDS (1 M, 16.54 mL) were mixed under N₂ atmosphere at 20° C. with stirring. The solution was cooled to −78° C. and a solution of benzyl (2R,4R)-4-methyl-5-oxo-2-phenyloxazolidine-3-carboxylate (5 g, 16.06 mmol) in THF (84 mL) was added dropwise. After stirring an additional 30 min, a solution of CH₂I₂ (3.89 mL, 48.18 mmol) in THF (33 mL) was added dropwise. The mixture was stirred at −78° C. for 90 min. The four batches were combined and warmed to 0° C. Sat. aq. NH₄Cl (100 mL) was added to the combined solution and the resulting mixture was extracted with EtOAc (2×100 mL). The combined EtOAc layers was washed with sat. aq. NH₄Cl (50 mL), H₂O (2×20 mL), and brine (30 mL), dried over Na₂SO₄, filtered, and concentrated under reduced pressure. The resulting residue was purified by silica gel column chromatography (1→17% EtOAc/pet. ether) to afford product (16 g, 55.2% yield).

### Step 3: Synthesis of methyl (R)-2-(((benzyloxy)carbonyl)amino)-3-iodo-2-methylpropanoate

To a mixture of benzyl (2R,4R)-4-(iodomethyl)-4-methyl-5-oxo-2-phenyloxazolidine-3-carboxylate (16 g, 35.46 mmol) in THF (90 mL) was added NaOMe (12.77 g, 70.91 mmol, 30% purity) dropwise over 10 min at −40° C. under N₂. The mixture was stirred at −40° C. for 2 h, then warmed to −20° C. and stirred for 1 h. The reaction was quenched by addition of H₂O (100 mL), and the resulting mixture was extracted with diethyl ether (3×100 mL). The combined organic layers were washed with brine (50 mL), dried over Na₂SO₄, filtered, and concentrated under reduced pressure. The resulting residue was purified by silica gel column chromatography (1→17% EtOAc/pet. ether) to afford product (10 g, 74.8% yield.

### Step 4: Synthesis of 1-benzyl 2-methyl (S)-2-methylaziridine-1,2-dicarboxylate

Four batches were completed in parallel. To a mixture of methyl (R)-2-(((benzyloxy)carbonyl)amino)-3-iodo-2-methylpropanoate (8 g, 21.20 mmol) in MeCN (800 mL) was added Ag₂O (14.76 g, 63.64 mmol) in one portion at 20° C. The mixture was stirred at 90° C. for 30 min. The four batches were combined, filtered, and concentrated under reduced pressure to afford product (5.1 g, 90.9% yield.

### Step 5: Synthesis of (S)-1-((benzyloxy)carbonyl)-2-methylaziridine-2-carboxylic acid

To a solution of 1-benzyl 2-methyl (S)-2-methylaziridine-1,2-dicarboxylate (1 g, 4.01 mmol) in MeCN (5 mL) was added a solution of NaOH (240.69 mg, 6.02 mmol) in H₂O (5 mL) at 0° C., then the mixture was stirred at 0° C. for 30 min. The mixture was lyophilized directly to afford crude product (1.05 g, crude). LCMS (ESI) m/z: [M+H] calcd for C₁₂H₁₂NO₄: 234.08; found 234.2.

### Intermediates A-108 and A-109. Synthesis of tert-butyl (R)-7-((S)-2-(benzyloxy)-1-cyclopentyl-2-oxoethyl)-6-oxo-2,7-diazaspiro[4.4]nonane-2-carboxylate and tert-butyl (S)-7-((S)-2-(benzyloxy)-1-cyclopentyl-2-oxoethyl)-6-oxo-2,7-diazaspiro[4.4]nonane-2-carboxylate

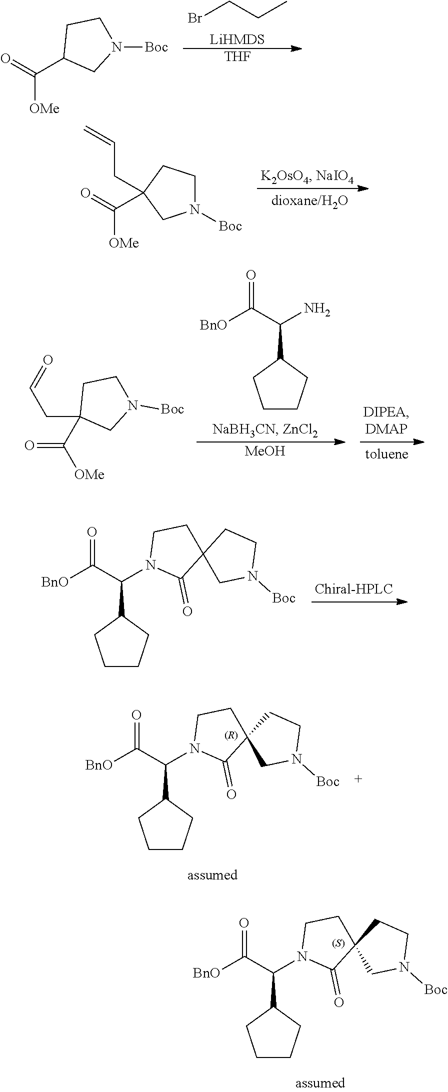

### Step 1: Synthesis of 1-(tert-butyl) 3-methyl 3-allylpyrrolidine-1,3-dicarboxylate

To a solution of 1-(tert-butyl) 3-methyl pyrrolidine-1,3-dicarboxylate (10.0 g, 43.6 mmol) in THF (100 mL) at −78° C. was added LiHMDS (65.0 mL, 65.4 mmol, 1 M in THF). After 1 h allyl bromide (5.63 mL, 65.4 mmol) was added and the resulting mixture was warmed to room temperature overnight. The reaction was quenched at 0° C. by the addition of NH₄Cl (200 mL). The aqueous layer was extracted with EtOAc (3×100 mL) and the combined organic layers were washed with brine, dried with Na₂SO₄, filtered, and concentrated under reduced pressure. Purification by normal phase chromatography (5% EtOAc/pet. ether) afforded the desired product (10.0 g, 76.6% yield).

### Step 3: Synthesis of 1-(tert-butyl) 3-methyl 3-(2-oxoethyl)pyrrolidine-1,3-dicarboxylate

To a solution of 1-(tert-butyl) 3-methyl 3-allylpyrrolidine-1,3-dicarboxylate (10.0 g, 37.1 mmol) and 2,6-dimethylpyridine (8.65 mL, 80.7 mmol) in dioxane (571 mL) and H₂O (142 mL) at 0° C. was added K₂OsO₄·2H₂O (0.27 g, 0.73 mmol). After 15 min NaIO₄ (23.82 g, 111.4 mmol) was added and the resulting mixture was stirred overnight at room temperature and then was diluted with H₂O (200 mL). The aqueous layer extracted with EtOAc (3×200 mL) and the combined organic layers were washed with 2 M HCl, dried with Na₂SO₄, filtered and concentrated under reduced pressure to afford the desired product (9.7 g, crude) which was used without further purification.

### Step 4: Synthesis of 1-(tert-butyl) 3-methyl 3-(2-(((S)-2-(benzyloxy)-1-cyclopentyl-2-oxoethyl)amino)ethyl)pyrrolidine-1,3-dicarboxylate

To a solution of 1-(tert-butyl) 3-methyl 3-(2-oxoethyl)pyrrolidine-1,3-dicarboxylate (9.60 g, 35.4 mmol) in MeOH (100 mL) at 0° C. was added benzyl (S)-2-amino-2-cyclopentylacetate (12.38 g, 53.075 mmol) and zinc chloride (7.23 g, 53.1 mmol). After 30 min NaBH₃CN (4.45 g, 70.8 mmol) was added and the resulting mixture stirred for 2 h at room temperature, concentrated under reduced pressure and the residue diluted with H₂O (150 mL). The aqueous layer was extracted with EtOAc (3×50 mL) and the combined organic layers were washed with brine, dried with Na₂SO₄, filtered, and then concentrated under reduced pressure. Purification by normal phase chromatography (20% EtOAc/pet. ether) afforded the desired product (11.1 g, 64.2% yield). LCMS (ESI) m/z: [M+H] calcd for C₂₇H₄₀N₂O₆: 489.30; found 489.3.

### Step 5: Synthesis of tert-butyl (R)-7-((S)-2-(benzyloxy)-1-cyclopentyl-2-oxoethyl)-6-oxo-2,7-diazaspiro[4.4]nonane-2-carboxylate and tert-butyl (S)-7-((S)-2-(benzyloxy)-1-cyclopentyl-2-oxoethyl)-6-oxo-2,7-diazaspiro[4.4]nonane-2-carboxylate

To a solution of stirred solution of 1-(tert-butyl) 3-methyl 3-(2-(((S)-2-(benzyloxy)-1-cyclopentyl-2-oxoethyl)amino)ethyl)pyrrolidine-1,3-dicarboxylate (11.1 g, 22.7 mmol) in toluene (120 mL) was added DIPEA (39.6 mL, 227 mmol) and DMAP (2.78 g, 22.7 mmol). The resulting mixture was stirred for 2 days at 80° C. and then concentrated under reduced pressure. Purification by reverse phase chromatography (20-70% MeCN/H₂O, 0.1% HCO₂H) afforded a mixture of desired products. The diastereomers were separated by prep-SFC (30% EtOH/CO₂) to afford tert-butyl (R)-7-((S)-2-(benzyloxy)-1-cyclopentyl-2-oxoethyl)-6-oxo-2,7-diazaspiro[4.4]nonane-2-carboxylate (3.73 g, 44.4% yield) LCMS (ESI) m/z: [M+H] calcd for C₂₆H₃₆N₂O₅: 457.27; found 457.3 and tert-butyl (S)-7-((S)-2-(benzyloxy)-1-cyclopentyl-2-oxoethyl)-6-oxo-2,7-diazaspiro[4.4]nonane-2-carboxylate (3.87 g, 46.1% yield)) LCMS (ESI) m/z: [M+H] calcd for C₂₆H₃₆N₂O₅: 457.27; found 457.3.

### Intermediate B-1. Synthesis of N-(3-(3-(4-methoxyphenyl)thioureido)propanoyl)-N-methyl-L-valine

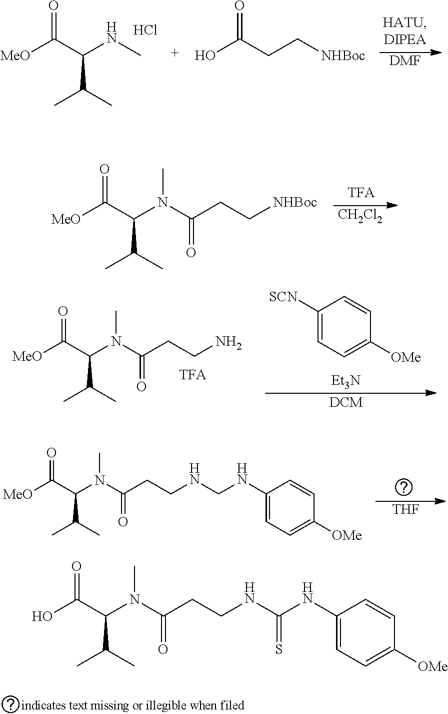

### Step 1: Synthesis of methyl N-(3-((tert-butoxycarbonyl)amino)propanoyl)-N-methyl-L-valinate

To a solution of 3-((tert-butoxycarbonyl)amino)propanoic acid (1.04 g, 5.50 mmol) in DMF (6 mL) was added DIPEA (2.38 mL, 13.7 mmol) followed by HATU (2.71 g, 7.15 mmol). The reaction mixture was stirred for 5 min and methyl methyl-L-valinate hydrochloride (1 g, 5.50 mmol) was added. The reaction was stirred at room temperature for 3 h and was then quenched with H₂O. The aqueous layer was extracted with EtOAc (3×10 mL) and the combined organic layers were washed with brine, and dried over Na₂SO₄, filtered, and concentrated under reduced pressure to afford the desired crude product.

### Step 2: Synthesis of methyl N-(3-aminopropanoyl)-N-methyl-L-valinate trifluoroacetic acid

To a solution of methyl N-(3-((tert-butoxycarbonyl)amino)propanoyl)-N-methyl-L-valinate (1.74 g, 5.50 mmol) in DCM (3 mL) was added TFA (2.09 mL, 27.4 mmol). The reaction was stirred at room temperature overnight and was then concentrated under reduced pressure to afford a solution of the desired crude product as a 33.5% solution in TFA.

### Step 3: Synthesis of methyl N-(3-(3-(4-methoxyphenyl)thioureido)propanoyl)-N-methyl-L-valinate

To a 33.5 wt % solution of methyl N-(3-aminopropanoyl)-N-methyl-L-valinate trifluoroacetic acid (800 mg, 0.811 mmol) in TFA was added DCM (5 mL) followed by Et₃N (593 μL, 4.26 mmol) and 4-methoxyphenyl isothiocyanate (117.0 μL, 852 μmol). The reaction was stirred at room temperature for 3 h. The reaction mixture was then washed with H₂O (2×5 mL), aq. NH₄Cl (5 mL), and brine (5 mL). The organic layer was dried over Na₂SO₄ and concentrated under reduced pressure to afford the crude product (290.2 mg 89.2% yield) as an oil, which was taken on without purification. LCMS (ESI) m/z: [M+H] calcd for C₁₈H₂₇N₃O₄S: 382.18; found 382.2.

### Step 4: Synthesis of N-(3-(3-(4-methoxyphenyl)thioureido)propanoyl)-M-methyl-L-valine

To a solution of methyl N-(3-(3-(4-methoxyphenyl)thioureido)propanoyl)-N-methyl-L-valinate (290.2 mg, 0.76 mmol) in THF (1 mL) was added a solution of LiOH·H₂O (41.4 mg, 0.99 mmol) in H₂O (300 μL). The reaction mixture was stirred overnight and was then acidified with HCl (4 M in dioxane, 120 μL, 0.48 mmol). The solution was then concentrated, the residue was dissolved in EtOAc, and the organic layer washed with H₂O (3×5 mL) and brine (5 mL). The organic layer was dried over Na₂SO₄ and concentrated under reduced pressure to afford the crude product (215.1 mg 77.0% yield), which was taken forward without further purification. LCMS (ESI) m/z: [M+H] calcd for C₁₇H₂₅N₃O₄S: 368.16; found 368.2.

The following table of compounds were prepared using the methods or variations thereof used to synthesize Intermediate B-1.

TABLE 3
Intermediate B

| | Molecular | | |
|---|---|---|---|
| Structure | Formula | Calculated MW | Observed MW |
| 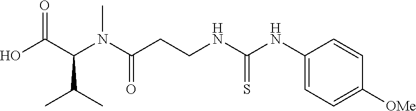 [SMILES: COc1ccc(NC(=S)NCCC(=O)N(C)[C@H](C(=O)O)C(C)C)cc1] | C₁₇H₂₅N₃O₄S | [M + H] = 368.16 | [M + H] = 368.5 |
| Intermediate B-1 | | | |
| 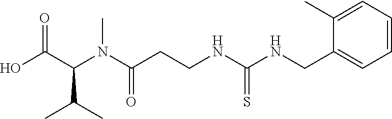 [SMILES: Cc1ccccc1CNC(=S)NCCC(=O)N(C)[C@H](C(=O)O)C(C)C] | C₁₈H₂₇N₃O₃S | [M + H] = 366.19 | [M + H] = 366.6 |
| Intermediate B-2 | | | |
| 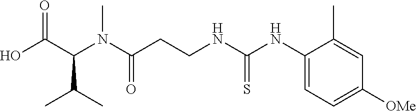 [SMILES: COc1ccc(NC(=S)NCCC(=O)N(C)[C@H](C(=O)O)C(C)C)c(C)c1] | C₁₈H₂₇N₃O₄S | [M + H] = 382.18 | [M + H] = 382.5 |
| Intermediate B-3 | | | |
| 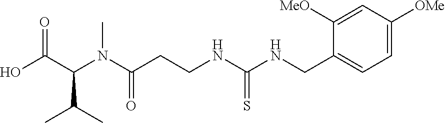 [SMILES: COc1ccc(CNC(=S)NCCC(=O)N(C)[C@H](C(=O)O)C(C)C)c(OC)c1] | C₁₉H₂₉N₃O₅S | [M + H] = 412.19 | [M + H] = 412.6 |
| Intermediate B-4 | | | |
| 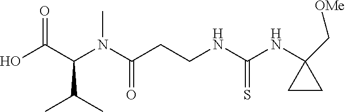 [SMILES: COCC1(NC(=S)NCCC(=O)N(C)[C@H](C(=O)O)C(C)C)CC1] | C₁₅H₂₇N₃O₄S | [M + H] = 346.18 | [M + H] = 346.6 |
| Intermediate B-5 | | | |
| 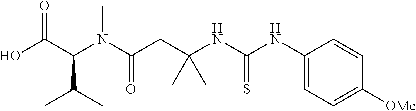 [SMILES: COc1ccc(NC(=S)NC(C)(C)CC(=O)N(C)[C@H](C(=O)O)C(C)C)cc1] | C₁₉H₂₉N₃O₄S | [M + H] = 396.20 | [M + H] = 396.6 |
| Intermediate B-6 | | | |
| 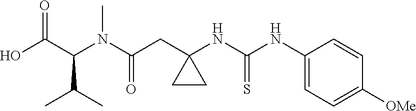 [SMILES: COc1ccc(NC(=S)NC2(CC(=O)N(C)[C@H](C(=O)O)C(C)C)CC2)cc1] | C₁₉H₂₇N₃O₄S | [M + H] = 394.18 | [M + H] = 394.7 |
| Intermediate B-7 | | | |
| 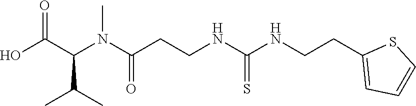 [SMILES: CC(C)[C@@H](C(=O)O)N(C)C(=O)CCNC(=S)NCCc1cccs1] | C₁₆H₂₅N₃O₃S₂ | [M + H] = 372.14 | [M + H] = 327.6 |
| Intermediate B-8 | | | |
| 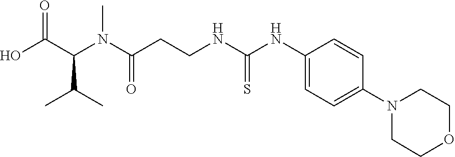 [SMILES: CC(C)[C@@H](C(=O)O)N(C)C(=O)CCNC(=S)Nc1ccc(N2CCOCC2)cc1] | C₂₀H₃₀N₄O₄S | [M + H] = 423.21 | [M + H] = 423.6 |
| Intermediate B-9 | | | |
| 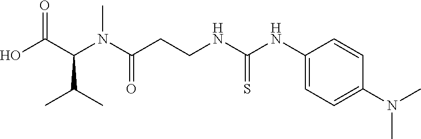 [SMILES: CC(C)[C@@H](C(=O)O)N(C)C(=O)CCNC(=S)Nc1ccc(N(C)C)cc1] | C₁₈H₂₈N₄O₃S | [M + H] = 381.20 | [M + H] = 381.6 |
| Intermediate B-10 | | | |
| 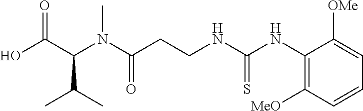 [SMILES: COc1cccc(OC)c1NC(=S)NCCC(=O)N(C)[C@H](C(=O)O)C(C)C] | C₁₈H₂₇N₃O₅S | [M + H] = 398.17 | [M + H] = 398.6 |
| Intermediate B-11 | | | |
| 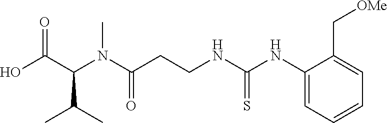 [SMILES: COCc1ccccc1NC(=S)NCCC(=O)N(C)[C@H](C(=O)O)C(C)C] | C₁₈H₂₇N₃O₄S | [M + H] = 382.18 | [M + H] = 382.6 |
| Intermediate B-12 | | | |
| 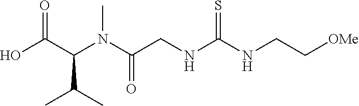 [SMILES: COCCNC(=S)NCC(=O)N(C)[C@H](C(=O)O)C(C)C] | C₁₂H₂₃N₃O₄S | [M + Na] = 328.13 | [M + Na] = 328.5 |
| Intermediate B-13 | | | |
| 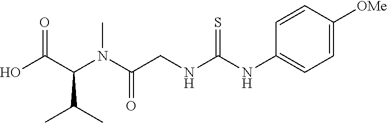 [SMILES: COc1ccc(NC(=S)NCC(=O)N(C)[C@H](C(=O)O)C(C)C)cc1] | C₁₆H₂₃N₃O₄S | [M + H] = 354.15 | [M + H] = 354.5 |
| Intermediate B-14 | | | |
| 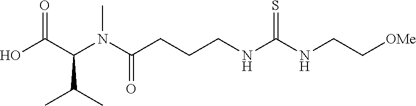 [SMILES: COCCNC(=S)NCCCC(=O)N(C)[C@H](C(=O)O)C(C)C] | C₁₄H₂₇N₃O₄S | [M + H] = 334.18 | [M + H] = 334.6 |
| Intermediate B-15 | | | |
| 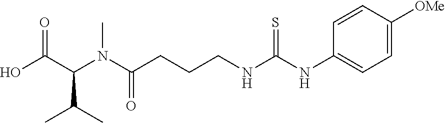 [SMILES: COc1ccc(NC(=S)NCCCC(=O)N(C)[C@H](C(=O)O)C(C)C)cc1] | C₁₈H₂₇N₃O₄S | [M + H] = 382.18 | [M + H] = 382.6 |
| Intermediate B-16 | | | |
| 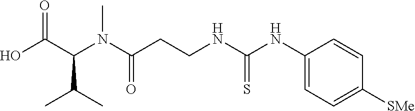 [SMILES: CSc1ccc(NC(=S)NCCC(=O)N(C)[C@H](C(=O)O)C(C)C)cc1] | C₁₇H₂₅N₃O₃S₂ | [M + H] = 384.14 | [M + H] = 384.3 |
| Intermediate B-17 | | | |
| 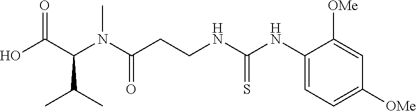 [SMILES: COc1ccc(NC(=S)NCCC(=O)N(C)[C@H](C(=O)O)C(C)C)c(OC)c1] | C₁₈H₂₇N₃O₅S | [M + H] = 398.17 | [M + H] = 398.4 |
| Intermediate B-18 | | | |
| 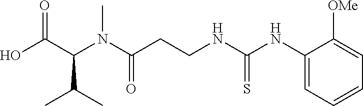 [SMILES: COc1ccccc1NC(=S)NCCC(=O)N(C)[C@H](C(=O)O)C(C)C] | C₁₇H₂₅N₃O₄S | [M + H] = 368.16 | [M + H] = 368.4 |
| Intermediate B-19 | | | |
| 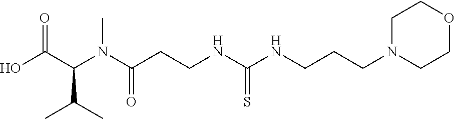 [SMILES: CC(C)[C@@H](C(=O)O)N(C)C(=O)CCNC(=S)NCCCN1CCOCC1] | C₁₇H₃₂N₄O₄S | [M + H] = 389.22 | [M + H] = 389.4 |
| Intermediate B-20 | | | |
| 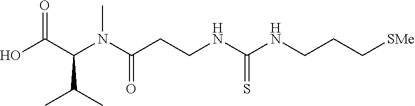 [SMILES: CSCCCNC(=S)NCCC(=O)N(C)[C@H](C(=O)O)C(C)C] | C₁₄H₂₇N₃O₃S₂ | [M + H] = 350.16 | [M + H] = 350.4 |
| Intermediate B-21 | | | |
| 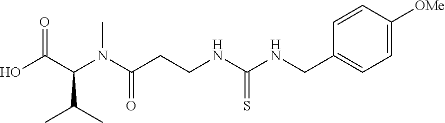 [SMILES: COc1ccc(CNC(=S)NCCC(=O)N(C)[C@H](C(=O)O)C(C)C)cc1] | C₁₈H₂₇N₃O₄S | [M + H] = 382.18 | [M + H] = 382.4 |
| Intermediate B-22 | | | |
| 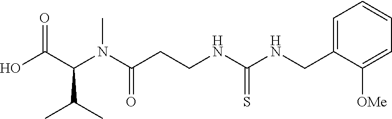 [SMILES: COc1ccccc1CNC(=S)NCCC(=O)N(C)[C@H](C(=O)O)C(C)C] | C₁₈H₂₇N₃O₄S | [M + H] = 382.18 | [M + H] = 382.4 |
| Intermediate B-23 | | | |
| 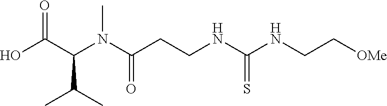 [SMILES: COCCNC(=S)NCCC(=O)N(C)[C@H](C(=O)O)C(C)C] | C₁₃H₂₅N₃O₄S | [M + H] = 320.16 | [M + H] = 320.3 |
| Intermediate B-24 | | | |
| 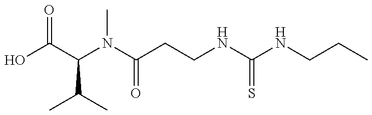 [SMILES: CCCNC(=S)NCCC(=O)N(C)[C@H](C(=O)O)C(C)C] | C₁₃H₂₅N₃O₃S | [M + H] = 304.17 | [M + H] = 304.5 |
| Intermediate B-25 | | | |
| 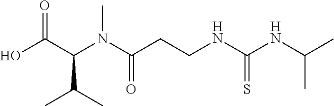 [SMILES: CC(C)NC(=S)NCCC(=O)N(C)[C@H](C(=O)O)C(C)C] | C₁₃H₂₅N₃O₃S | [M + H] = 304.17 | [M + H] = 304.3 |
| Intermediate B-26 | | | |
| 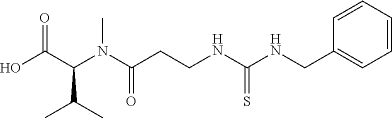 [SMILES: CC(C)[C@@H](C(=O)O)N(C)C(=O)CCNC(=S)NCc1ccccc1] | C₁₇H₂₅N₃O₃S | [M + H] = 352.17 | [M + H] = 352.4 |
| Intermediate B-27 | | | |
| 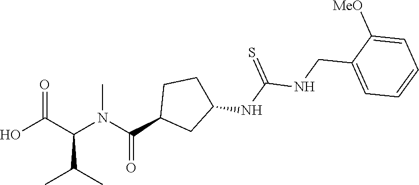 [SMILES: COc1ccccc1CNC(=S)N[C@H]1CC[C@H](C(=O)N(C)[C@H](C(=O)O)C(C)C)C1] | C₂₁H₃₁N₃O₄S | [M + H] = 422.21 | [M + H] = 422.7 |
| Intermediate B-28 | | | |
| 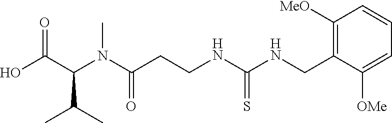 [SMILES: COc1cccc(OC)c1CNC(=S)NCCC(=O)N(C)[C@H](C(=O)O)C(C)C] | C₁₉H₂₉N₃O₅S | [M + H] = 412.19 | [M + H] = 412.2 |
| Intermediate B-29 | | | |
| 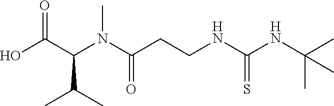 [SMILES: CC(C)[C@@H](C(=O)O)N(C)C(=O)CCNC(=S)NC(C)(C)C] | C₁₄H₂₇N₃O₃S | [M + H] = 318.19 | [M + H] = 318.6 |
| Intermediate B-30 | | | |
| 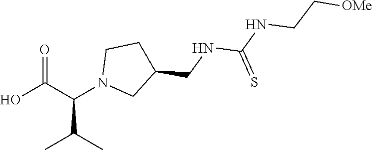 [SMILES: COCCNC(=S)NC[C@@H]1CCN([C@H](C(=O)O)C(C)C)C1] | C₁₄H₂₇N₃O₃S | [M + H] = 318.19 | [M + H] = 318.7 |
| Intermediate B-31 | | | |
| 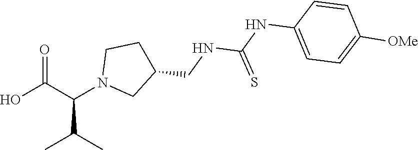 [SMILES: COc1ccc(NC(=S)NC[C@H]2CCN([C@H](C(=O)O)C(C)C)C2)cc1] | C₁₈H₂₇N₃O₃S | [M + H] = 366.19 | [M + H] = 366.7 |
| Intermediate B-32 | | | |
| 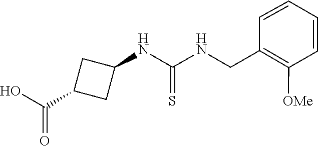 [SMILES: COc1ccccc1CNC(=S)N[C@H]1C[C@H](C(=O)O)C1] | C₁₄H₁₈N₂O₃S | [M + H] = 295.11 | [M + H] = 295.6 |
| Intermediate B-33 | | | |
| 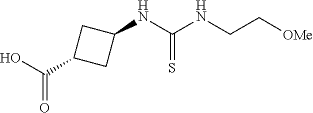 [SMILES: COCCNC(=S)N[C@H]1C[C@H](C(=O)O)C1] | C₉H₁₆N₂O₃S | [M + H] = 233.10 | [M + H] = 233.4 |
| Intermediate B-34 | | | |

### Example 1. Synthesis of (3S)-1-((R)-aziridine-2-carbonyl)-N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide

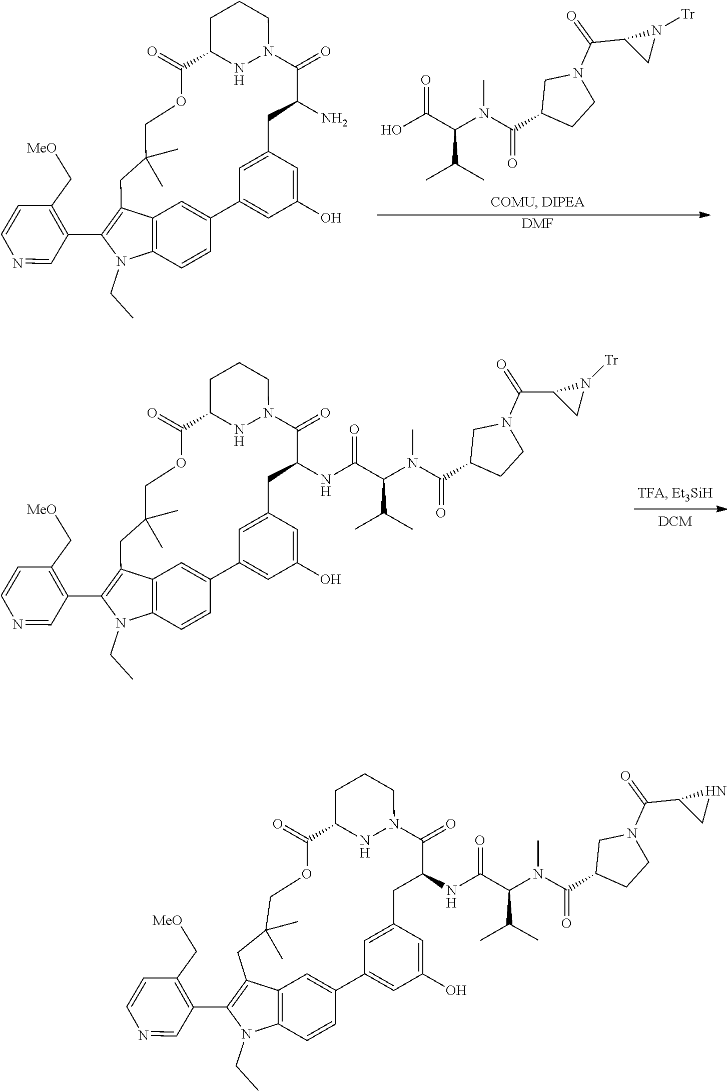

### Step 1: Synthesis of (3S)—N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-((R)-1-tritylaziridine-2-carbonyl)pyrrolidine-3-carboxamide

To a solution of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (520.0 mg, 0.831 mmol) and N-methyl-N—((S)-1-((R)-1-tritylaziridine-2-carbonyl)pyrrolidine-3-carbonyl)-L-valine (0.6727 g, 1.25 mmol) in DMF (10 mL) at 0° C. was added COMU (0.5338 mg, 1.25 mmol) followed by DIPEA (1.16 mL, 6.65 mmol). After 2 h, the reaction mixture was extracted with EtOAc (3×100 mL) and the combined organic layers were washed with brine (3×30 mL), dried over Na₂SO₄, filtered, and concentrated under reduced pressure. The crude residue was purified by reverse phase chromatography (10→50% MeCN/H₂O) to afford the desired product (500 mg, 52.4% yield) as a solid. LCMS (ESI) m/z: [M+H] calcd for C₆₉H₇₈N₈O₈: 1147.60; found 1147.8.

### Step 2: Synthesis of (3S)-1-((R)-aziridine-2-carbonyl)-N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide

To a stirred solution of (3S)—N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-((R)-1-tritylaziridine-2-carbonyl)pyrrolidine-3-carboxamide (145.0 mg, 0.126 mmol) in DCM (3 mL) at 0° C. was added Et₃SiH (58.8 mg, 0.505 mmol) followed by TFA (57.6 mg, 0.505 mmol). After 1 h, DIPEA was added to the reaction mixture until pH 8. The resulting mixture was concentrated under reduced pressure, and the residue was purified by reverse phase chromatography (10→50% MeCN/H₂O) to afford the desired product (70 mg, 61.2% yield) as a solid. LCMS (ESI) m/z: [M+H] calcd for C₅₀H₆₄N₈O₆: 905.49; found 905.7.

### Example 7. Synthesis of (2R)—N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl-N-methylaziridine-2-carboxamide

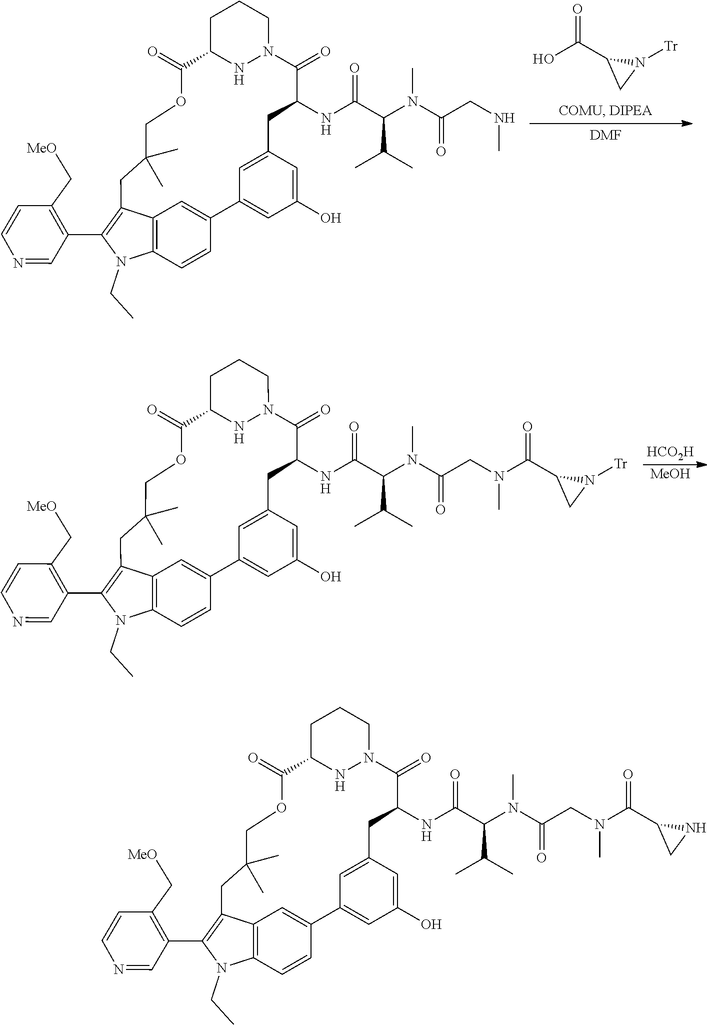

### Step 1: Synthesis of (2R)—N-(2-(((2S)-1-(((6³S,4S)-1-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)amino)-2-oxoethyl)-N-methyl-1-tritylaziridine-2-carboxamide

To a solution of (2S)—N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(N-methyl-2-(methylamino)acetamido)butanamide (285.7 mg, 0.353 mmol) in DMF (3.0 mL) at 0° C. was added (R)-1-tritylaziridine-2-carboxylic acid (232.4 mg, 0.705 mmol) followed by DIPEA (0.61 mL, 4.7 mmol) and COMU (211.4 mg, 0.494 mmol). The resulting mixture was warmed to room temperature and stirred for 1 h. The reaction mixture was diluted with H₂O (15 mL) and the mixture was extracted with EtOAc (3×4 mL). The combined organic layers were washed with brine (10 mL), dried over Na₂SO₄, filtered, and concentrated under reduced pressure. The residue was purified by prep-TLC (12% EtOAc/pet. ether) to afford the desired product (301 mg, 68% yield) as a solid. LCMS (ESI) m/z: [M+H] calcd for C₆₇H₇₆N₆O₈: 1121.59; found 1121.8.

### Step 2: Synthesis of (2R)—N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a solution of (2R)—N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)amino)-2-oxoethyl)-N-methyl-1-tritylaziridine-2-carboxamide (301.0 mg, 0.268 mmol) in MeOH (3.0 mL) at 0° C. was added HCO₂H (1.50 mL). The reaction mixture was stirred for 1 h and then neutralized to pH 8 with DIPEA. The resulting mixture was diluted with H₂O (15 mL) and extracted with EtOAc (3×4 mL). The combined organic layers were dried over Na₂SO₄, filtered, and concentrated under reduced pressure. The residue was purified by reverse phase chromatography (30→60% MeCN/H₂O) to afford the desired product (89.9 mg, 38% yield) as a solid. LCMS (ESI) m/z: [M+H] calcd for C₄₈H₆₂N₈O₈: 879.48; found 879.7.

### Example 15. Synthesis of two Isomers, 15A and 15B, of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(3-((((4-methoxyphenyl)imino)methylene)amino)-N-methylpropanamido)-3-methylbutanamide

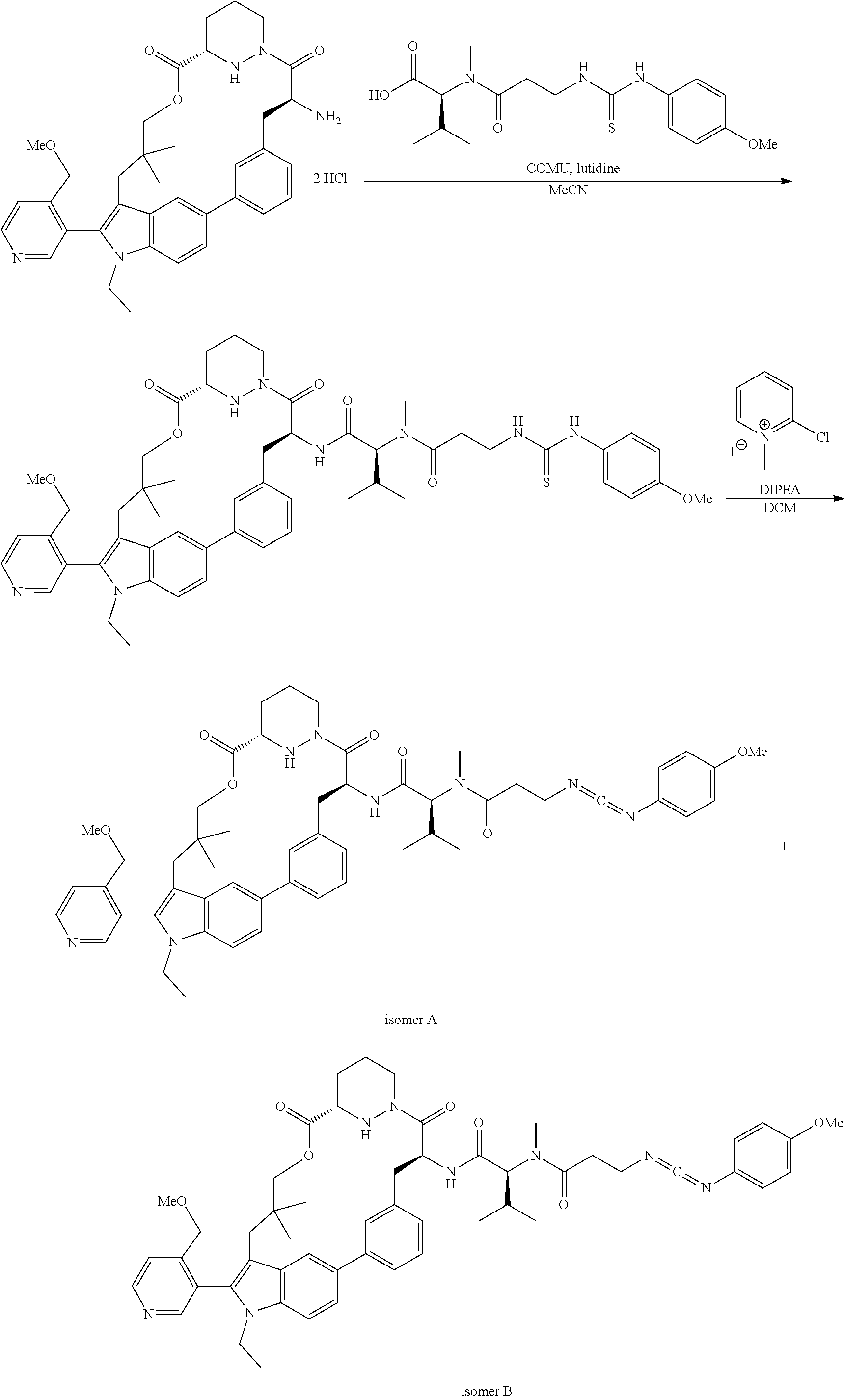

### Step 1: Synthesis of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(3-(3-(4-methoxyphenyl)thioureido)-N-methylpropanamido)-3-methylbutanamide

To a solution of (6³S,4S)-4-amino-1¹-ethyl-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (108 mg, 168 μmol) and N-(3-(3-(4-methoxyphenyl)thioureido)propanoyl)-N-methyl-L-valine (61.9 mg, 168 μmol) in MeCN (2 mL) at 0° C. was added 2,6-lutidine (97.8 μL, 840 μmol) followed by COMU (78.8 mg, 184 μmol). After 1 h at 0° C. the reaction was diluted with EtOAc and the organic portion washed with H₂O (15 mL) and brine (15 mL), dried over Na₂SO₄, and concentrated under reduced pressure. Purification by silica gel chromatography (20→100% EtOAc/Hex then 0-5% MeOH/EtOAc) afforded the desired product (117.0 mg 72.6% yield). LCMS (ESI) m/z: [M+H] calcd for C₅₃H₆₆N₈O₇S: 959.49; found 959.5.

### Step 2: Synthesis of two isomers of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-(3-((((4-methoxyphenyl)imino)methylene)amino)-N-methylpropanamido)-3-methylbutanamide

To a solution of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(3-(3-(4-methoxyphenyl)thioureido)-N-methylpropanamido)-3-methylbutanamide (117.0 mg, 121 μmol) in DCM (1 mL) was added DIPEA (63.2 μL, 363 μmol) followed by 2-chloro-1-methylpyridin-1-ium iodide (42.6 mg, 181 μmol). The reaction mixture was stirred overnight, at which point the solid was filtered and the crude solution was purified by reverse phase chromatography (40→100 MeCN/H₂O+0.4% NH₄OH) to afford two separated isomers as the desired earlier eluting isomer 15A (6.9 mg, 6.2% yield) and later eluting isomer 15B (2.5 mg, 2.2% yield). LCMS (ESI) m/z: [M+H] calcd for C₅₃H₆₄N₈O₇: 925.50; found 925.5 and LCMS (ESI) m/z: [M+H] calcd for C₅₃H₆₄N₈O₇: 925.50; found 925.6.

### Example 25. Synthesis of (2S)-2-(3-(3-(2-chloroethyl)ureido)-N-methylpropanamido)-N-((6³S,4S)-1¹-ethyl-2⁵-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

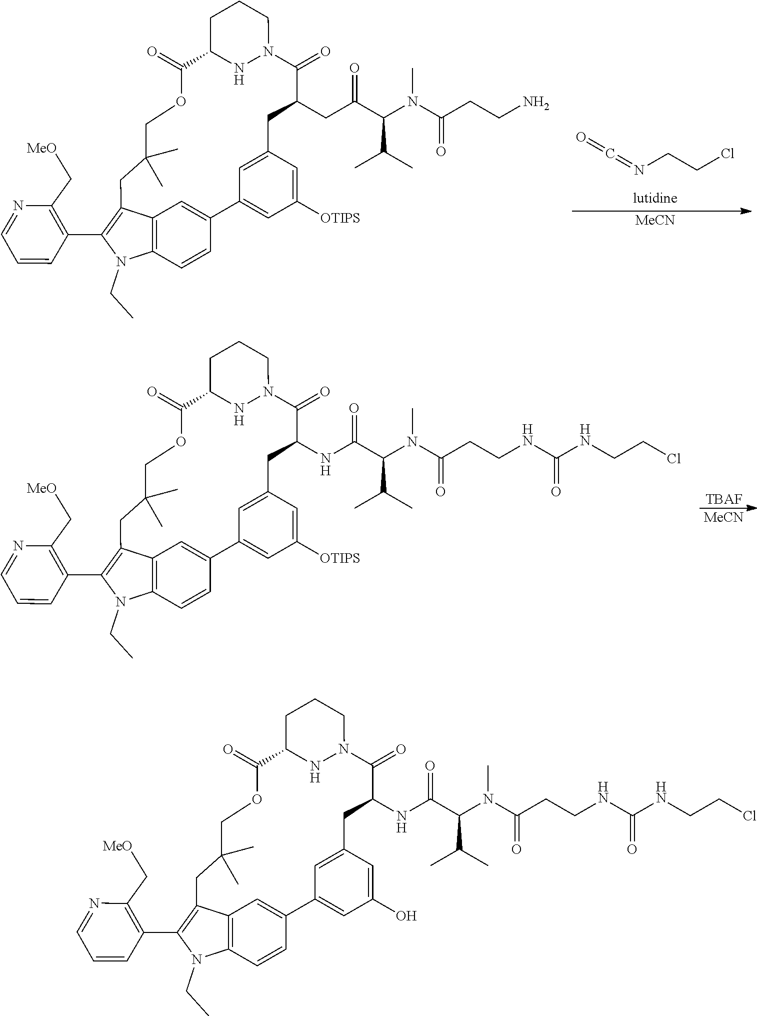

### Step 1: Synthesis of (2S)-2-(3-(3-(2-chloroethyl)ureido)-N-methylpropanamido)-N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)-2-(3-amino-N-methylpropanamido)-N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (106 mg, 109 μmol) in MeCN (544 μL) at 0° C. was added 1-chloro-2-isocyanatoethane (9.29 μL, 109 μmol) followed by Et₃N (15.1 μL, 109 μmol). After 12 min, the reaction was diluted with DCM (10 mL) and a solution of 1% formic acid in H₂O (10 mL). The aqueous layer was extracted with DCM (10 mL) and the combined organic layers were dried over Na₂SO₄, filtered, and then concentrated under reduced pressure to afford the desired product (117 mg, 100% yield), which was used in the next step without purification. LCMS (ESI) m/z: [M+H] calcd for C₅₇H₈₃ClN₈O₈Si: 1071.59; found 1071.5.

### Step 2: Synthesis of (2S)-2-(3-(3-(2-chloroethyl)ureido)-N-methylpropanamido)-N-((6³S,4S)-1¹-ethyl-2⁵-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)-2-(3-(3-(2-chloroethyl)ureido)-N-methylpropanamido)-N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (117 mg, 109 μmol) in MeCN (1.1 mL) at 0° C. was added TBAF (1M in dioxane, 109 μL, 109 μmol). After 5 min, the reaction was concentrated under reduced pressure and the crude residue was purified by normal phase chromatography (20→100% B/A, B=10% MeOH/EtOAc, A=hexanes) followed by reverse phase chromatography (20→60/o MeCN/H₂O) to afford the final product (82.2 mg, 82% yield). LCMS (ESI) m/z: [M+H] calcd for C₄₆H₆₃ClN₈O₈: 915.45; found 915.7.

### Example 30. Synthesis of (2S)-2-(3-((4,5-dihydrooxazol-2-yl)amino)-N-methylpropanamido)-N-((6³S,4S)-1¹-ethyl-2⁵-hydroxy-1²-(2-(methoxymethyl)pyridin-3-yl)-10,10-dimethyl-5,7-dioxo-6¹,6²6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

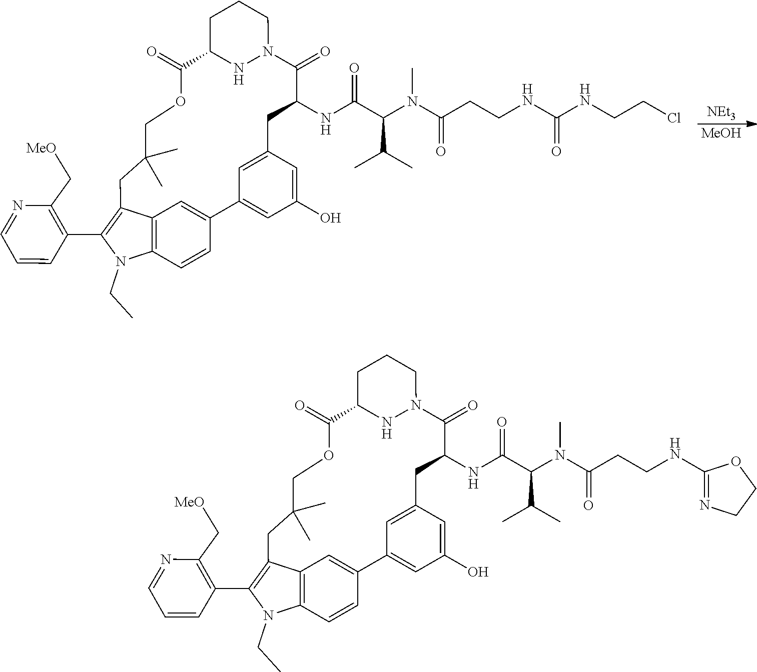 [SMILES: "MeOH" ; CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2 ; CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2 ; "NEt3"]

A solution of (2S)-2-(3-(3-(2-chloroethyl)ureido)-N-methylpropanamido)-N-((6³S,4S)-1¹-ethyl-2⁵-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (55.0 mg, 60.0 μmol) and Et₃N (25.1 μL, 180 μmol) in MeOH (1.2 mL) was heated in the microwave at 150° C. for 1 min. The reaction mixture was cooled to room temperature and concentrated under reduced pressure. The crude residue was then purified by reverse phase chromatography (30→100% MeCN/H₂O+0.4% NH₄OH) to afford the final product (21.1 mg, 40% yield). LCMS (ESI) m/z: [M+H] calcd for C₄₈H₆₂N₈O₈: 879.48; found 879.4.

### Example 31. Synthesis of (3S)—N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-((S)-oxirane-2-carbonyl)pyrrolidine-3-carboxamide

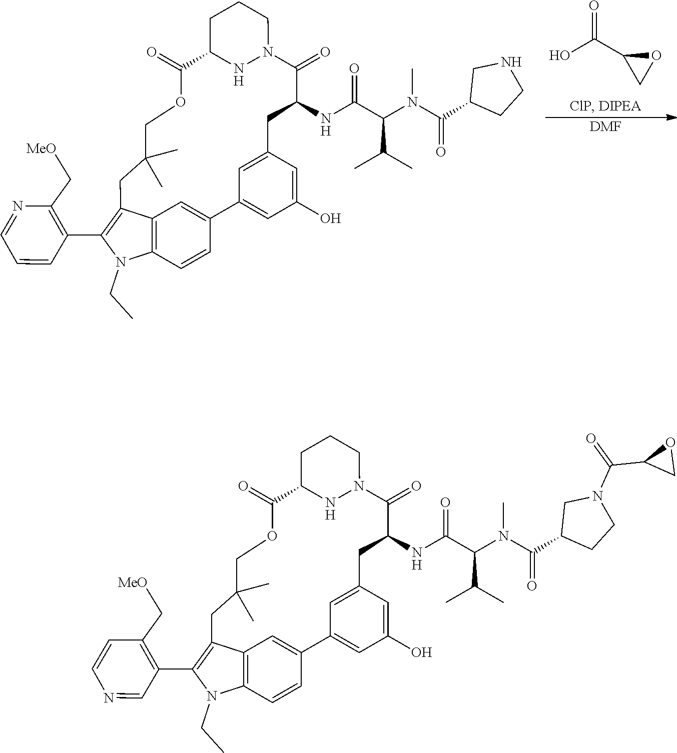

To a solution of potassium (S)-oxirane-2-carboxylate (16.98 mg, 0.135 mmol), 2-chloro-1,3-dimethylimidazolidinium hexafluorophosphate (87.46 mg, 0.314 mmol), and DIPEA (0.156 mL, 0.897 mmol) in DMF (1.5 mL) at 0° C. was added (3S)—N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (75.0 mg, 0.09 mmol). The resulting mixture was stirred overnight at room temperature, at which point it was diluted with EtOAc (100 mL). The organic layer was washed with brine (3×5 mL), dried over Na₂SO₄, filtered, and concentrated under reduced pressure. Purification by reverse phase chromatography (25→55% MeCN/H₂O) afforded the desired product (6.3 mg, 7.8% yield) as a solid. LCMS (ESI) m/z: [M+H] calcd for C₅₀H₆₃N₇O₉: 906.48; found 906.7.

### Example 34. Synthesis of (2R)-1-acetyl-N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

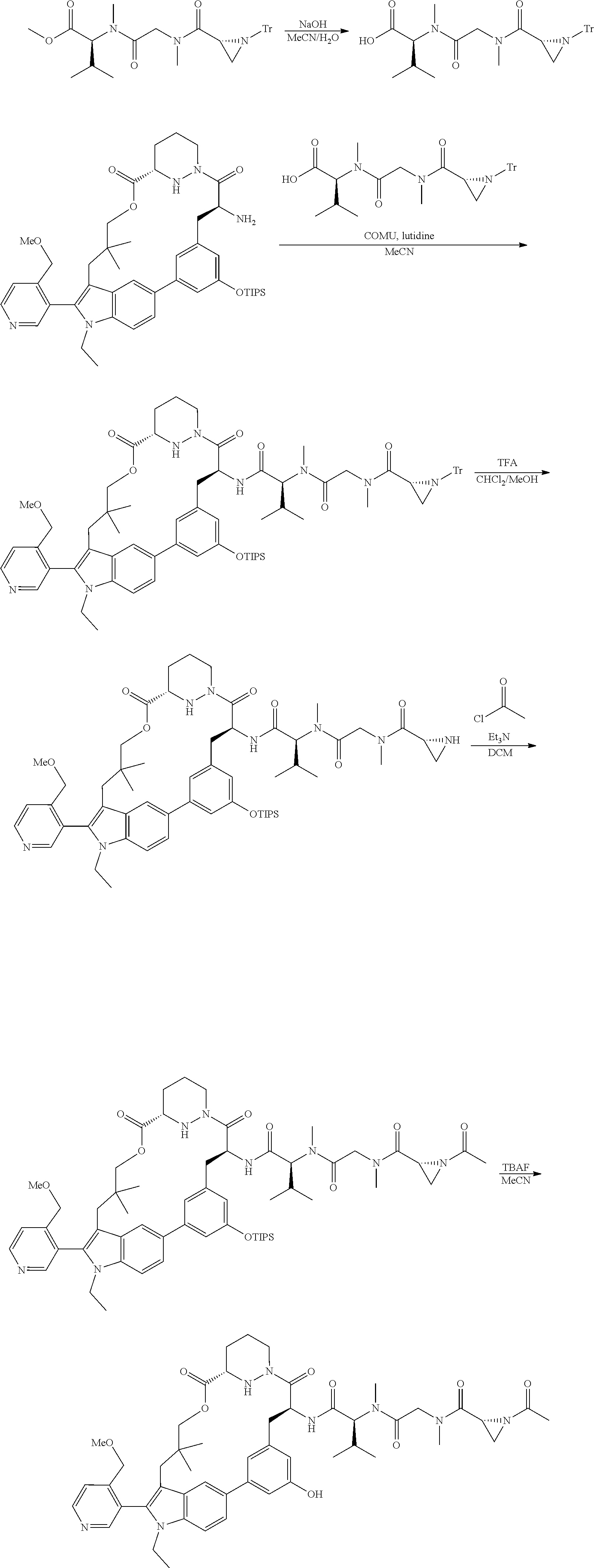

### Step 1: Synthesis of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-tritylaziridine-2-carboxamide

To a solution of (6³S,4S)-4-amino-1¹-ethyl-1²-(4-(methoxymethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (260 mg, 0.332 mmol) and N-methyl-N—(N-methyl-N—((R)-1-tritylaziridine-2-carbonyl)glycyl)-L-valine (204 mg, 0.399 mmol) in MeCN (3.3 mL) at 0° C. was added lutidine (192 μL, 1.66 mmol) followed by COMU (156 mg, 0.366 mmol). The reaction stirred at 0° C. for 1 h and was then diluted with EtOAc. The mixture was washed with H₂O/brine (1:1), dried over Na₂SO₄, filtered, and concentrated under reduced pressure. Purification by normal phase chromatography (0→100% EtOAc/hexanes) afforded the desired product (116 mg, 27% yield). LCMS (ESI) m/z: [M+H] calcd for C₇₆H₉₆N₈O₈Si: 1277.72; found 1277.7.

### Step 2: Synthesis of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-tritylaziridine-2-carboxamide (400 mg, 0.313 mmol) in MeOH (1.56 mL) and chloroform (1.56 mL) at 0° C. was added TFA (191 μL, 2.50 mmol). The reaction stirred at 0° C. for 2 h and was then quenched with lutidine (364 μL, 3.13 mmol). The reaction mixture was diluted with DCM, washed with H₂O, and concentrated under reduced pressure. Purification by reverse phase chromatography (10→100% MeCN/H₂O) afforded the desired product (100 mg, 31% yield). LCMS (ESI) m/z: [M+H] calcd for C₅₇H₈₂N₈O₈Si: 1035.61; found 1035.6.

### Step 3: Synthesis of (2R)-1-acetyl-N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a solution of (2R)—N-(2-(((2S)-1-(((6³S,4S)-1-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide (33 mg, 0.032 mmol) in DCM (637 μL) at 0° C. was added Et₃N (22.1 μL, 0.159 mmol) followed by acetyl chloride (4.54 μL, 0.064 mmol). The reaction stirred at 0° C. for 1 h. The reaction was then diluted with DCM, washed with NaHCO₃, dried over Na₂SO₄, filtered, and concentrated under reduced pressure to afford the desired crude product (37 mg, 100% yield). LCMS (ESI) m/z: [M+H] calcd for C₅₉H₈₄N₆O₉Si: 1077.62; found 1077.6.

### Step 4: Synthesis of (2R)-1-acetyl-N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a solution of (2R)-1-acetyl-N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide (34 mg, 0.032 mmol) in MeCN (631 μL) at 0° C. was added TBAF (1M in THF, 31.5 μL, 0.032 mmol). The reaction stirred for 10 min and was then diluted with DCM, washed with brine, dried over Na₂SO₄, filtered, and concentrated under reduced pressure. Purification by reverse phase chromatography (10→100% MeCN/H₂O) afforded the desired product (8.5 mg, 29% yield). LCMS (ESI) m/z: [M+H] calcd for C₅₀H₆₄N₈O₉: 921.49; found 921.5.

### Example 36. Synthesis of (2R)—N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(methylsulfonyl)aziridine-2-carboxamide

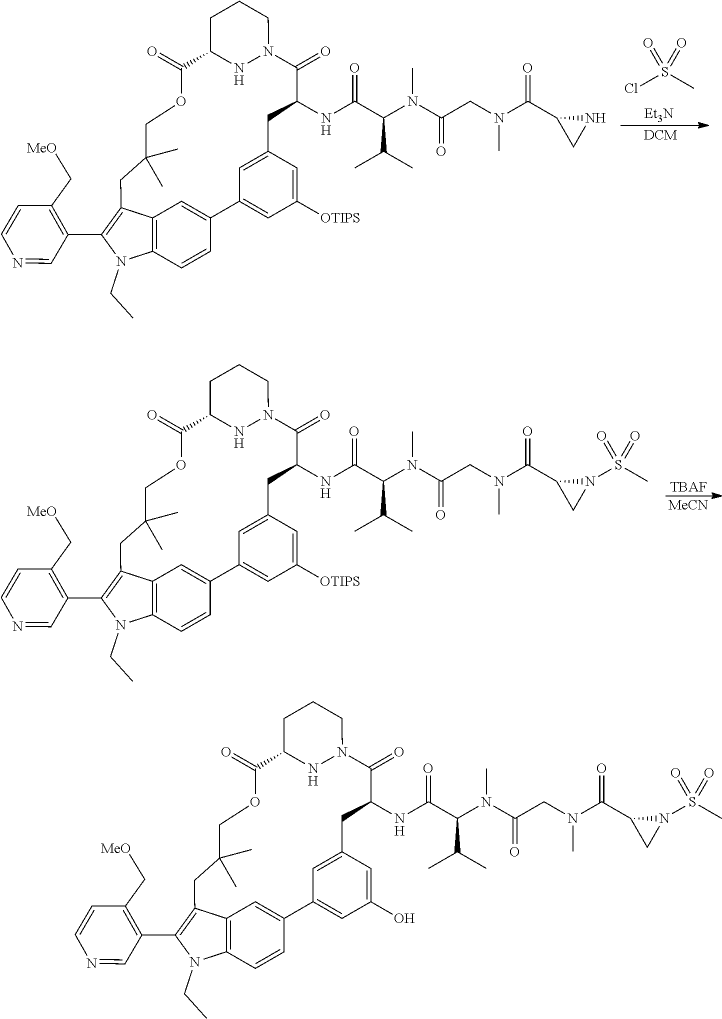

### Step 1: Synthesis of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(methylsulfonyl)aziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide (33 mg, 0.032 mmol) in DCM (637 μL) at 0° C. was added Et₃N (22.1 μL, 0.159 mmol) followed by methanesulfonyl chloride (4.93 μL, 0.064 mmol). The reaction was cooled to 0° C. for 1 h and was then diluted with DCM, washed with NaHCO₃, dried over Na₂SO₄, filtered, and concentrated under reduced pressure to afford the desired crude product (35 mg, 100% yield). LCMS (ESI) m/z: [M+H] calcd for C₅₈H₈₄N₈O₁₀SSi: 1113.59; found 1113.6.

### Step 2: Synthesis of (2R)—N-(2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(methylsulfonyl)aziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(methylsulfonyl)aziridine-2-carboxamide (35 mg, 0.032 mmol) in MeCN (646 μL) at 0° C. was added TBAF (1M in THF, 32.3 μL, 0.032 mmol). The reaction stirred for 10 min and was then diluted with DCM, washed with brine, dried over Na₂SO₄, filtered, and concentrated under reduced pressure. Purification by reverse phase chromatography (10→100% MeCN/H₂O) afforded the desired product (20 mg, 65% yield). LCMS (ESI) m/z: [M+H] calcd for C₄₉H₆₄N₈O₁₀S: 957.45; found 957.5.

### Example 38. Synthesis of methyl (2R)-2-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)methyl)carbamoyl)aziridine-1-carboxylate

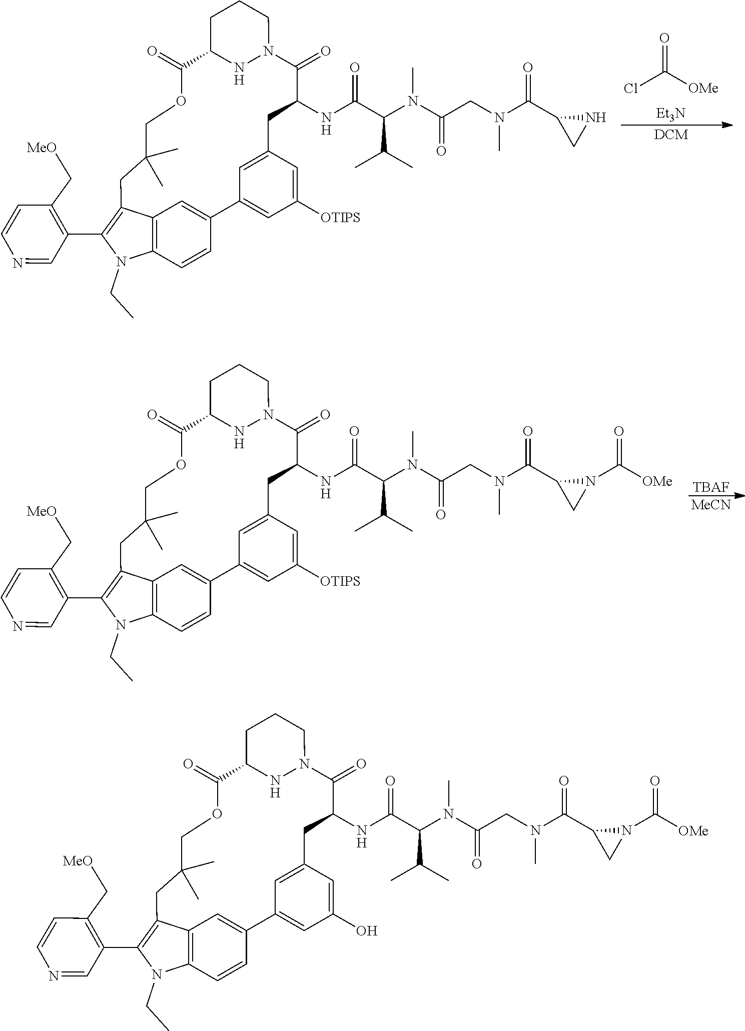

### Step 1: Synthesis of methyl (2R)-2-((2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-1-carboxylate

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide (46 mg, 0.044 mmol) in DCM (888 μL) at 0° C. was added E₃N (30.8 μL, 0.22 mmol) followed by methyl chloroformate (4.46 μL, 0.058 mmol). The reaction stirred at 0° C. for 1 h and then the reaction was diluted with DCM, washed with NaHCO₃, dried over Na₂SO₄, filtered, and concentrated under reduced pressure to afford the desired crude product (56 mg, 100% yield). LCMS (ESI) m/z: [M+H] calcd for C₅₉H₈₄N₈O₁₀Si: 1093.62; found 1093.7.

### Step 2: Synthesis of methyl (2R)-2-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-1-carboxylate

To a solution of methyl (2R)-2-((2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-1-carboxylate (56 mg, 0.051 mmol) in MeCN (1.0 mL) at 0° C. was added TBAF (1M in THF, 51.2 μL, 0.051 mmol). The reaction stirred for 15 min and was then diluted with DCM, washed with brine, dried over Na₂SO₄, filtered, and concentrated under reduced pressure. Purification by reverse phase chromatography (10→100% MeCN/H₂O) afforded the desired product (17 mg, 36% yield). LCMS (ESI) m/z: [M+H] calcd for C₅₀H₆₄N₈O₁₀: 937.48; found 937.6.

### Example 48 and 49. Synthesis of methyl (2S,3R)-1-((R)-tert-butylsulfinyl)-3-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-2-carboxylate and methyl (2S,3R)-3-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-2-carboxylate

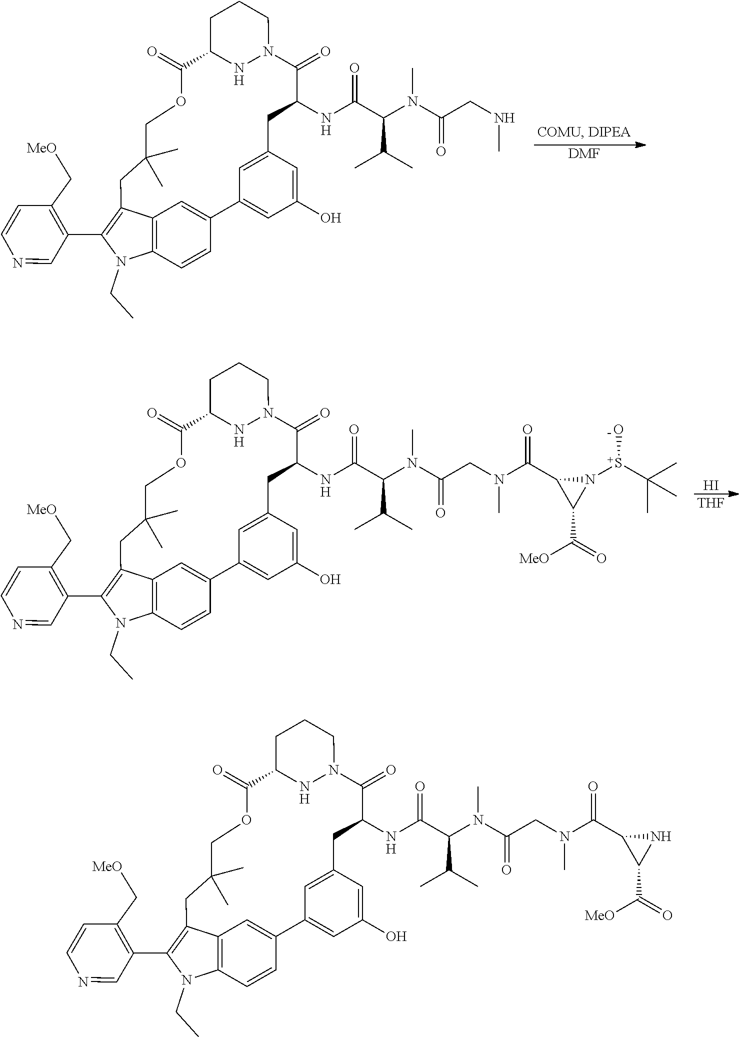

### Step 1: Synthesis of methyl (2S,3R)-1-((R)-tert-butylsulfinyl)-3-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-2-carboxylate

To a solution of (2S)—N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(N-methyl-2-(methylamino)acetamido)butanamide (267.0 mg, 0.33 mmol) and (2R,3S)-1-((R)-tert-butylsulfinyl)-3-(methoxycarbonyl)aziridine-2-carboxylic acid (246.5 mg, 0.99 mmol) in DMF (4.5 mL) at 0° C. was added DIPEA (0.574 mL, 3.3 mmol) followed by a solution of COMU (211.8 mg, 0.49 mmol) in DMF (0.5 mL). The resulting mixture was stirred for 1 h at 0° C. and was then quenched with sat. NH₄Cl. The aqueous layer was extracted with EtOAc (3×20 mL) and the combined organic layers were washed with brine (2×50 mL), dried over Na₂SO₄, filtered, concentrated under reduced pressure. The crude product was purified by reverse phase chromatography (35→65% MeCN/H₂O) to afford the desired product (253 mg, 73.7% yield). LCMS (ESI) m/z: [M+H] calcd for C₅₄H₇₂N₈O₁₁S: 1041.51; found 1041.8.

### Step 2: Synthesis of methyl (2S,3R)-3-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-2-carboxylate

To a solution of methyl (2S,3R)-1-((R)-tert-butylsulfinyl)-3-((2-(((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)(methyl)carbamoyl)aziridine-2-carboxylate (200.0 mg, 0.19 mmol) in THF (4.0 mL) at 0° C. was added HI (1.0 mL), dropwise. The resulting mixture was stirred for 10 min at 0° C. and was then basified to pH 7 with DIPEA. The mixture was extracted with EtOAc (3×30 mL) and the combined organic layers were washed with brine (50 mL), dried over Na₂SO₄, filtered, and concentrated under reduced pressure. The residue was purified by reverse phase chromatography (35→,65% MeCN/H₂O) to afford the desired product (13.2 mg, 7.3% yield). LCMS (ESI) m/z: [M+H] calcd for C₅₀H₆₄N₈O₁₀: 937.48; found 938.6.

### Example 55. Synthesis of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

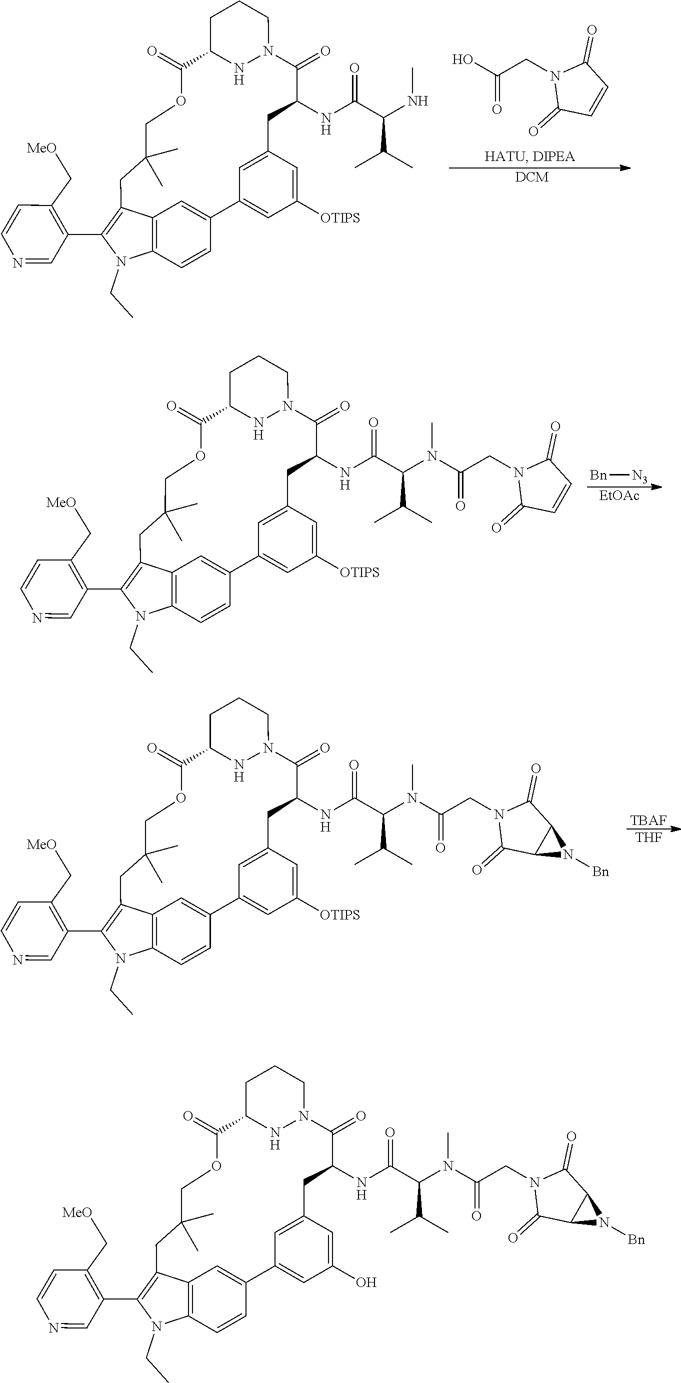

### Step 1: Synthesis of (2S)-2-(2-(2,5-dioxo-2,5-dihydro-1H-pyrrol-1-yl)-N-methylacetamido)-N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)—N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (600.0 mg, 0.67 mmol) and 2-(2,5-dioxo-2,5-dihydro-1H-pyrrol-1-yl)acetic acid (124.7 mg, 0.80 mmol) in DCM (6.0 mL) at 0° C. was added DIPEA (0.934 mL, 5.36 mmol) followed by HATU (382.2 mg, 1.01 mmol). The reaction mixture was warmed to room temperature and stirred for 3 h. The reaction was then quenched by the addition of H₂O (20 mL). The aqueous layer was extracted with DCM (2×50 mL) and the combined organic layers were washed with brine (2×50 mL), dried over Na₂SO₄, and concentrated under reduced pressure. The residue was purified by normal phase chromatography (10→20% EtOAc/pet. ether) to afford the desired product (260 mg, 33.8% yield). LCMS (ESI) m/z: [M+H] calcd for C₅₇H₇₇N₇O₉Si: 1032.56; found 1032.8.

### Step 2: Synthesis of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)-2-(2-(2,5-dioxo-2,5-dihydro-1H-pyrrol-1-yl)-N-methylacetamido)-N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (250.0 mg, 0.24 mmol) in EtOAc (2.0 mL) was added (azidomethyl)benzene (80.6 mg, 0.61 mmol). The reaction mixture was heated to 80° C. and stirred for 2 h. The reaction mixture was then heated to 120° C. and stirred for 2 days. The reaction mixture was then cooled to room temperature and quenched with H₂O. The aqueous layer was extracted with EtOAc and the combined organic layers were washed with brine, dried with Na₂SO₄, filtered, and concentrated under reduced pressure. The residue was purified by normal phase chromatography to afford the desired product (50 mg, 18.1% yield). LCMS (ESI) m/z: [M+H] calcd for C₆₄H₈₄N₈O₉Si: 1137.62; found 1138.3.

### Step 3: Synthesis of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (50.0 mg, 0.04 mmol) in THF (0.5 mL) at 0° C. was added 1M TBAF (0.07 mL, 0.07 mmol). The reaction mixture was stirred for 1 h. The reaction mixture was then diluted with H₂O and extracted with EtOAc. The combined organic layers were washed with brine, dried over Na₂SO₄, filtered, and concentrated under reduced pressure. The residue was purified by prep-TLC followed by reverse phase chromatography (45→72% MeCN/H₂O) to afford the desired product (20 mg, 46.4% yield). LCMS (ESI) m/z: [M+Na] calcd for C₅₅H₆₄N₈O₉: 1003.47; found 1003.8.

### Example 95. Synthesis of (2R)—N-(2-(((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methylaziridine-2-carboxamide

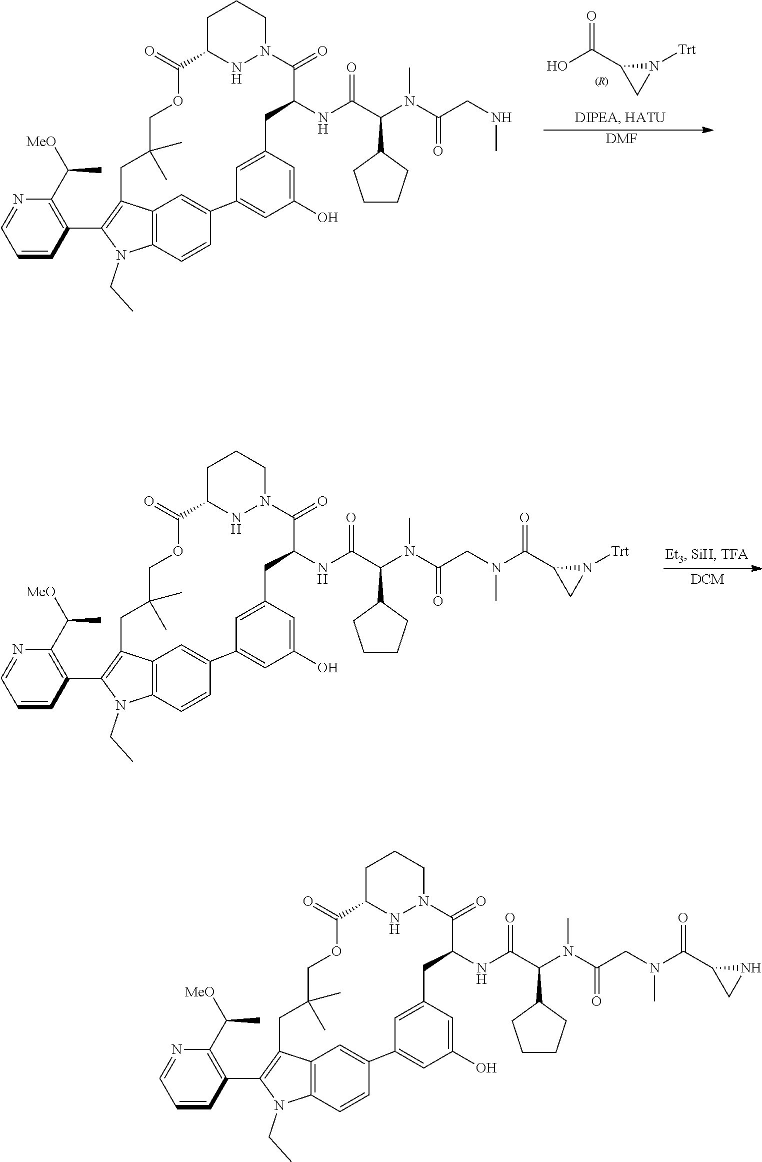

### Step 1: Synthesis of (2R)—N-(2-(((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methyl-1-tritylaziridine-2-carboxamide

To a mixture of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(N-methyl-2-(methylamino)acetamido)acetamide (321.2 mg, 0.276 mmol), DIPEA (0.472 mL, 2.764 mmol), and (R)-1-tritylaziridine-2-carboxylic acid (136.59 mg, 0.415 mmol) in DMF (3.0 mL) at 0° C. was added HATU (126.14 mg, 0.332 mmol). The resulting mixture was stirred at 0° C. for 30 min, then diluted with H₂O (30 mL) and extracted with EtOAc (3×30 mL). The combined organic layers were washed with brine (3×10 mL), dried with Na₂SO₄, filtered, and concentrated under reduced pressure. Purification by prep-TLC (50% EtOAc/pet. ether) afforded the desired product (200 mg, 62.3% yield). LCMS (ESI) m/z: [M+H] calcd for C₇₀H₈₀N₈O₈: 1161.62; found 1161.5.

### Step 2: Synthesis of (2R)—N-(2-(((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methylaziridine-2-carboxamide

To a mixture of (2R)—N-(2-(((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methyl-1-tritylaziridine-2-carboxamide (195.0 mg, 0.168 mmol) in DCM (2.0 mL) at 0° C. was added Et₃SiH (78.09 mg, 0.672 mmol) and TFA (76.57 mg, 0.672 mmol). The resulting mixture was stirred at 0° C. for 30 min then basified to pH 8 with DIPEA and concentrated under reduced pressure. Purification by reverse phase chromatography (25→55% MeCN/H₂O) to afford the desired product (60 mg, 38.9/a yield). LCMS (ESI) m/z: [M+H] calcd for C₅₁H₆₆N₈O₈: 919.51; found 919.5.

### Example 87. Synthesis of 6-((S)-aziridin-2-yl)-N-((2S)-1-(((6'S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylnicotinamide

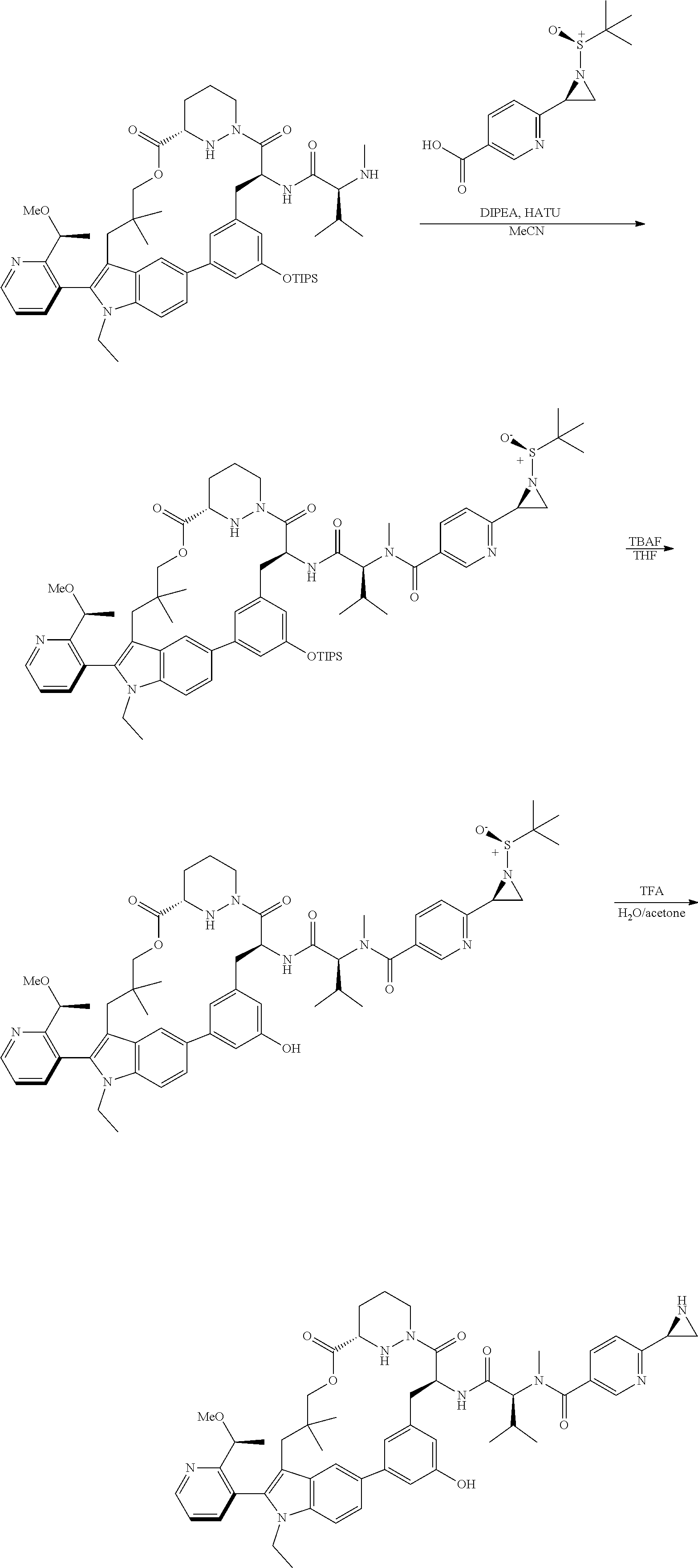

### Step 1: Synthesis of 6-((2S)-1-(tert-butylsulfinyl)aziridin-2-yl)-N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylnicotinamide

To a mixture of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (198.24 mg, 0.218 mmol) and DIPEA (0.074 mL, 0.436 mmol) in MeCN (10 mL) at 0° C. was added HATU (200 mg, 0.526 mmol) and the resulting mixture was stirred for 3 min. To the mixture was then added a solution of 6-((2S)-1-(tert-butylsulfinyl)aziridin-2-yl)nicotinic acid (117.0 mg, 0.436 mmol) in MeCN (10 mL) in portions. The resulting mixture was stirred overnight at 0° C. and then was then quenched with H₂O extracted with EtOAc (3×20 mL). The combined organic layers were washed with brine (20 mL), dried with Na₂SO₄, filtered, and concentrated under reduced pressure to afford the desired product (430 mg, 85.0% yield). LCMS (ESI) m/z: [M+H] calcd for C₆₄H₉₀N₈O₈SSi: 1159.65; found 1159.8.

### Step 2: Synthesis of 6-((2S)-1-(tert-butylsulfinyl)aziridin-2-yl)-N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylnicotinamide

To a solution of 6-((2S)-1-(tert-butylsulfinyl)aziridin-2-yl)-N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylnicotinamide (430.0 mg, 0.371 mmol) in THF (50.0 mL) at 0° C. was added TBAF (1 M in THF, 1.1 mL, 1.11 mmol) in portions. The resulting mixture was stirred at 0° C. for 2 h and was then concentrated under reduced pressure. The residue was purified by prep-TLC (5% MeOH/DCM) to afford the desired product (290 mg, 78% yield). LCMS (ESI) m/z: [M+H] calcd for C₅₅H₇₀N₈O₈S: 1003.51; found 1003.8.

### Step 3: Synthesis of 6-((S)-aziridin-2-yl)-N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylnicotinamide

To a solution of 6-((2S)-1-(tert-butylsulfinyl)aziridin-2-yl)-N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylnicotinamide (150.0 mg, 0.150 mmol) in H₂O (15.0 mL) and acetone (15.0 mL) at 0° C. was added TFA (7.50 mL, 100.97 mmol) in portions. The resulting mixture was warmed to room temperature and stirred for 48 h and then was neutralized to pH 8 with sat. NaHCO₃. The aqueous layer was extracted with EtOAc, dried with Na₂SO₄, filtered, and concentrated under reduced pressure. Purification by reverse phase chromatography (38→58% MeCN/H₂O) afforded the desired product (10.0 mg, 7.4% yield). LCMS (ESI) m/z: [M+H] calcd for C₅₁H₆₂N₈O₇: 899.48; found 899.5.

### Example 139. Synthesis of (2S)-2-((S)-7-(((R)-aziridin-2-yl)methyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

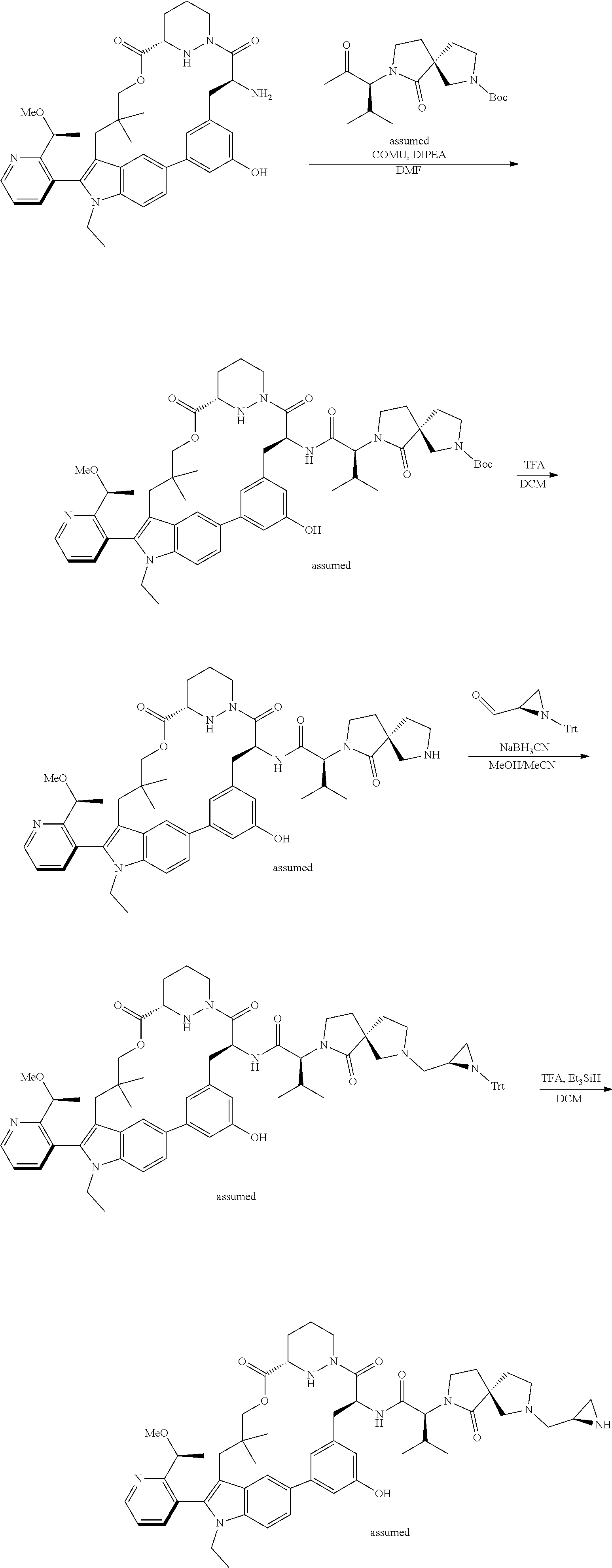

### Step 1: Synthesis of tert-butyl (5R)-7-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-6-oxo-2,7-diazaspiro[4.4]nonane-2-carboxylate

To a solution of (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (600 mg, 0.94 mmol) and DIPEA (820 μL, 4.7 mmol) in DMF (8 mL) at 0° C. was added (S)-2-((R)-7-(tert-butoxycarbonyl)-1-oxo-2,7-diazaspiro[4.4]nonan-2-yl)-3-methylbutanoic acid (380 mg, 1.13 mmol) and COMU (440 mg, 1.03 mmol). The reaction mixture was stirred for 1 h then was diluted with H₂O (100 mL) and extracted with EtOAc (3×50 mL). The combined organic layers were washed with brine (30 mL), dried with Na₂SO₄, filtered, and the filtrate was concentrated under reduced pressure. Purification by Prep-TLC (EtOAc) afforded the desired product (600 mg, 66% yield). LCMS (ESI) m/z: [M+H] calcd for C₅₄H₇₁N₇O₉: 962.54; found 962.5.

### Step 2: Synthesis of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-((S)-1-oxo-2,7-diazaspiro[4.4]nonan-2-yl)butanamide

To a solution of tert-butyl (5R)-7-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-6-oxo-2,7-diazaspiro[4.4]nonane-2-carboxylate (600 mg, 0.62 mmol) in DCM (6 mL) at 0° C. was added TFA (3.0 mL, 40 mmol). The reaction mixture was stirred for 2 h and then was concentrated under reduced pressure. The residue was diluted with H₂O (100 mL), basified to pH 8 with sat. aq. NaHCO₃, and extracted with EtOAc (3×60 mL). The combined organic layers were washed with brine (30 mL), dried with Na₂SO₄, filtered, and concentrated under reduced pressure to afford the desired product (430 mg, 79% yield). LCMS (ESI) m/z: [M+H] calcd for C₄₉H₆₃N₇O₇: 862.49; found 862.5.

### Step 3: Synthesis of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-((S)-1-oxo-7-(((S)-1-tritylaziridine-2-yl)methyl)-2,7-diazaspiro[4.4]nonan-2-yl)butanamide

To a solution of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-((S)-1-oxo-2,7-diazaspiro[4.4]nonan-2-yl)butanamide (200 mg, 0.23 mmol) and (R)-1-tritylaziridine-2-carbaldehyde (110 mg, 0.35 mmol) in MeOH (0.50 mL) and MeCN (4.0 mL) was added NaBH₃CN (29 mg, 0.46 mmol). The reaction mixture was stirred for 2 h then was quenched with sat. aq. NH₄Cl and was extracted with EtOAc (3×50 mL). The combined organic layers were washed with brine (30 mL), dried with Na₂SO₄, filtered, and concentrated under reduced pressure. Purification by Prep-TLC (EtOAc) afforded desired product (145 mg, 53% yield). LCMS (ESI) m/z: [M+H] calcd for C₇₁H₈₂N₈O₇: 1159.64; found 1159.6.

### Step 4: Synthesis of (2S)-2-((S)-7-(((R)-aziridin-2-yl)methyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-((S)-1-oxo-7-(((S)-1-tritylaziridine-2-yl)methyl)-2,7-diazaspiro[4.4]nonan-2-yl)butanamide (140 mg, 0.12 mmol) in DCM (2.0 mL) 0° C. was added TFA (74 μL, 0.97 mmol) and Et₃SiH (150 μL, 0.97 mmol). The reaction mixture was stirred for 30 min then was basified to pH 8 with DIPEA. The resulting mixture was concentrated under reduced pressure. Purification by reverse phase chromatography (30→60% MeCN/H₂O) afforded the desired product (37.5 mg, 31% yield). LCMS (ESI) m/z: [M+H] calcd for C₅₂H₆₈N₈O₇: 917.53; found 917.4.

### Example 133. Synthesis of (2R,3R)-3-cyclopropyl-N-(2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

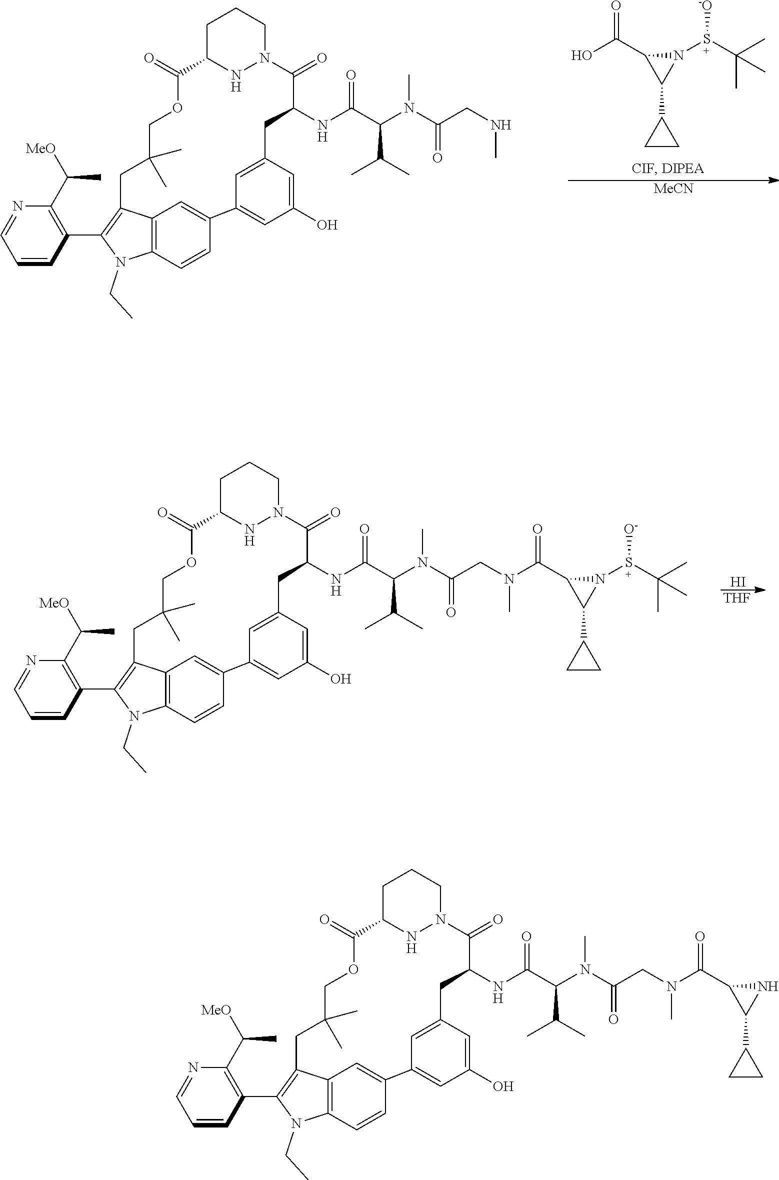

### Step 1: Synthesis of (2R,3R)-1-(tert-butylsulfinyl)-3-cyclopropyl-N-(2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a solution of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-(N-methyl-2-(methylamino)acetamido)butanamide (50 mg, 61 μmol) and (2R,3R)-1-(tert-butylsulfinyl)-3-cyclopropylaziridine-2-carboxylic acid (21 mg, 91 μmol) in MeCN at 0° C. was added DIPEA (210 μL, 1.2 mmol) and CIP (25 mg, 91 μmol). The resulting mixture was stirred for 2 h and was then concentrated under reduced pressure. Purification by Prep-TLC (9% EtOAc/pet. ether) afforded the desired product (270 mg, 54% yield). LCMS (ESI) m/z: [M+H] calcd for C₅₆H₇₆N₈O₉S: 1037.56; found 1037.4.

### Step 2: Synthesis of (2R,3R)-3-cyclopropyl-N-(2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a mixture of (2R,3R)-1-(tert-butylsulfinyl)-3-cyclopropyl-N-(2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide (230 mg, 0.22 mmol) in THF at 0° C. was added HI (0.50 mL, 3.8 mmol, 57% wt in H₂O). The reaction mixture was stirred for 10 min and then neutralized to pH 8 with DIPEA and concentrated under reduced pressure. Purification by reverse phase chromatography (40→60% MeCN/H₂O) afforded desired product (20 mg, 11% yield) as a white solid. LCMS (ESI) m/z: [M+H] calcd for C₅₂H₆₈N₈O₈: 933.53; found 933.6.

### Example 177. Synthesis of 4-((R)-aziridine-2-carbonyl)-N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperazine-1-carboxamide

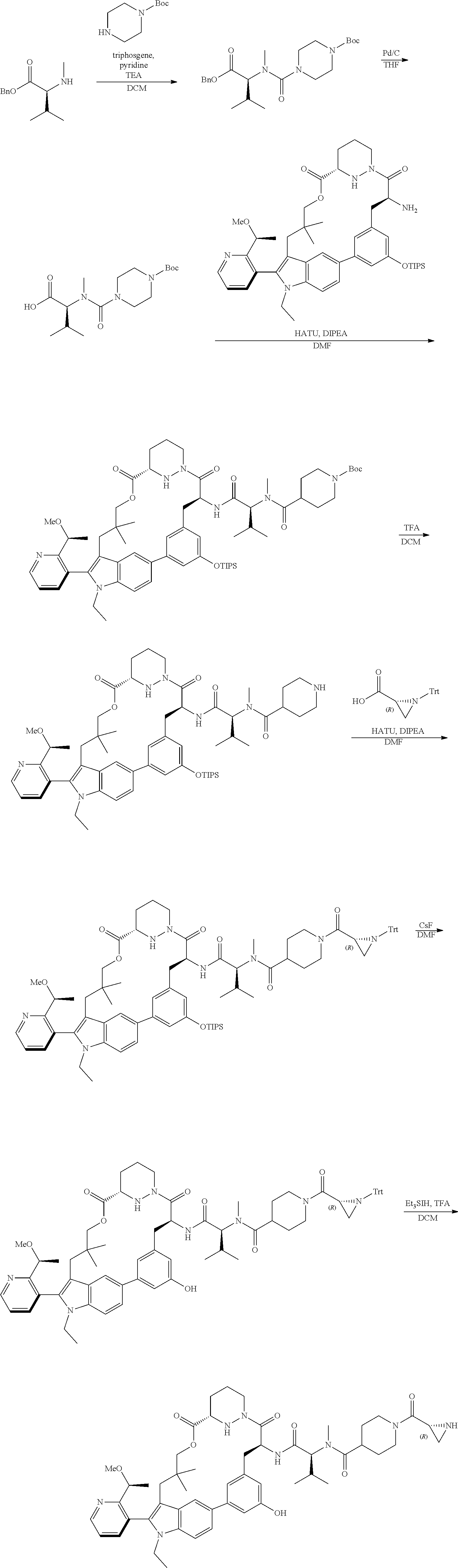

### Step 1: Synthesis of tert-butyl (S)-4-((1-(benzyloxy)-3-methyl-1-oxobutan-2-yl)(methyl)carbamoyl)piperazine-1-carboxylate

Into a 100-mL vial were added benzyl methyl-L-valinate (2.0 g, 9.038 mmol) and triphosgene (0.89 g, 2.982 mmol) in DCM (30 mL) followed by pyridine (2.14 g, 27.113 mmol) in portions at 0° C. under an N₂ atmosphere. The mixture was stirred for 2 h at room temperature. The crude product was used in the next step directly without further purification. Then, the resulting mixture was added to tert-butyl piperazine-1-carboxylate (2.22 g, 11.912 mmol) in DCM (25 mL) and Et₃N (2.78 g, 27.489 mmol) in portions at room temperature under an N₂ atmosphere. The resulting mixture was concentrated under reduced pressure. The residue was purified by silica gel column chromatography, (30% EtOAc/pet. ether) to afford the desired product (3.6 g, 90.6% yield). LCMS (ESI) m/z: [M+H] calcd for C₂₃H₃₅N₃O₅: 434.26; found 434.2.

### Step 2: Synthesis of N-(4-(tert-butoxycarbonyl)piperazine-1-carbonyl)-N-methyl-L-valine

Into a 100-mL vial were added tert-butyl (S)-4-((1-(benzyloxy)-3-methyl-1-oxobutan-2-yl)(methyl)carbamoyl) piperazine-1-carboxylate (2.95 g, 6.804 mmol) and Pd/C (1.48 g) in THF (25 mL). the reaction was stirred for overnight at room temperature under a hydrogen atmosphere. The resulting mixture was filtered, the filter cake was washed with EtOAc (3×50 mL), and the combined organic layers were concentrated under reduced pressure to afford the desired product (2.4 g, crude). LCMS (ESI) m/z: [M+H] calcd for C₁₆H₂₉N₃O₅: 344.21; found 344.4.

### Step 3: Synthesis of tert-butyl 4-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperazine-1-carboxylate

Into a 50-mL vial was added (6³S,4S)-4-amino-1¹-ethyl-1²-(2-((S)-1-methoxyethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.0 g, 1.256 mmol) and N-(4-(tert-butoxycarbonyl) piperazine-1-carbonyl)-N-methyl-L-valine (647.04 mg, 1.884 mmol) in DMF (8 mL) followed by HATU (668.63 mg, 1.758 mmol) and DIPEA (811.69 mg, 6.280 mmol) in portions at room temperature. The resulting mixture was extracted with EtOAc (3×50 mL). The combined organic layers were washed with brine (2×20 mL), dried over Na₂SO₄, filtered, and concentrated under reduced pressure. The residue was purified by silica gel column chromatography (50% EtOAc/pet. ether) to afford the desired product (1.08 g, 76.7% yield). LCMS (ESI) m/z: [M+H] calcd for C₆₂H₉₂N₈O₉Si: 1121.68; found 1122.0.

### Step 4: Synthesis of N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperazine-1-carboxamide

Into a 100-mL vial was added tert-butyl 4-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperazine-1-carboxylate (1.08 g, 0.963 mmol) and TFA (3.0 mL, 40.39 mmol) in DCM (12 mL). The reaction was stirred for 2 h at room temperature under an N₂ atmosphere. The resulting mixture was concentrated under reduced pressure to afford the desired product (907 mg, crude). LCMS (ESI) m/z: [M+Na] calcd for C₅₇H₈₃N₈O₇Si: 1042.61; found 1043.9.

### Step 5: Synthesis of N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-4-((R)-1-tritylaziridine-2-carbonyl)piperazine-1-carboxamide

Into a 40-mL vial was added N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperazine-1-carboxamide (400.0 mg, 0.392 mmol) and (R)-1-tritylaziridine-2-carboxylic acid (193.49 mg, 0.587 mmol) in DMF (3.5 mL) followed by HATU (208.46 mg, 0.548 mmol) and DIPEA (253.06 mg, 1.958 mmol) in portions at room temperature under an N₂ atmosphere. The resulting mixture was extracted with EtOAc (3×60 mL) and the combined organic layers were washed with brine (2×10 mL), dried over Na₂SO₄, filtered, and concentrated under reduced pressure. The residue was purified by silica gel column chromatography, (50% EtOAc/pet. ether) to afford the desired product (367 mg, 70.3% yield). LCMS (ESI) m/z: [M+H−TIPS] calcd for C₇₉H₁₀₁N₉O₈Si: 1176.63; found 1176.2.

### Step 6: Synthesis of N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)-N-methyl-4-((R)-1-tritylaziridine-2-carbonyl)piperazine-1-carboxamide

Into a 100-mL vial was added N-((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-4-((R)-1-tritylaziridine-2-carbonyl)piperazine-1-carboxamide (161.0 mg, 0.121 mmol) and CsF (91.75 mg, 0.604 mmol) in DMF (1.5 mL). The reaction was stirred for 2 h at room temperature and was then extracted with EtOAc (3×20 mL). The combined organic layers were washed with brine (2×10 mL), dried over Na₂SO₄, filtered, and concentrated under reduced pressure. The residue was purified by Prep-TLC (50% EtOAc/pet. ether) to afford the desired product (101 mg, 71.1% yield). LCMS (ESI) m/z: [M+H] calcd for C₇₀H₈₁N₉O₈: 1176.62; found 1176.9.

### Step 7: Synthesis of 4-((R)-aziridine-2-carbonyl)-N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperazine-1-carboxamide

Into a 40-mL vial was added N-((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-4-((R)-1-tritylaziridine-2-carbonyl) piperazine-1-carboxamide (101.0 mg, 0.086 mmol) and Et₃SiH (49.91 mg, 0.429 mmol) in DCM (2.0 mL) was added TFA (48.94 mg, 0.429 mmol) in portions at room temperature under an N₂ atmosphere. The mixture was basified to pH 8 with DIPEA. The crude product was purified by Prep-HPLC to afford the desired product (29.6 mg, 36.9% yield). LCMS (ESI) m/z: [M+H] calcd for C₅₁H₆₇N₉O₈: 934.51; found 934.3.

### Example 175. Synthesis of (2S)-2-((S)-7-((R)-aziridine-2-carbonyl)-1-oxo-2,7-diazaspiro[4.4]nonan-2-yl)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

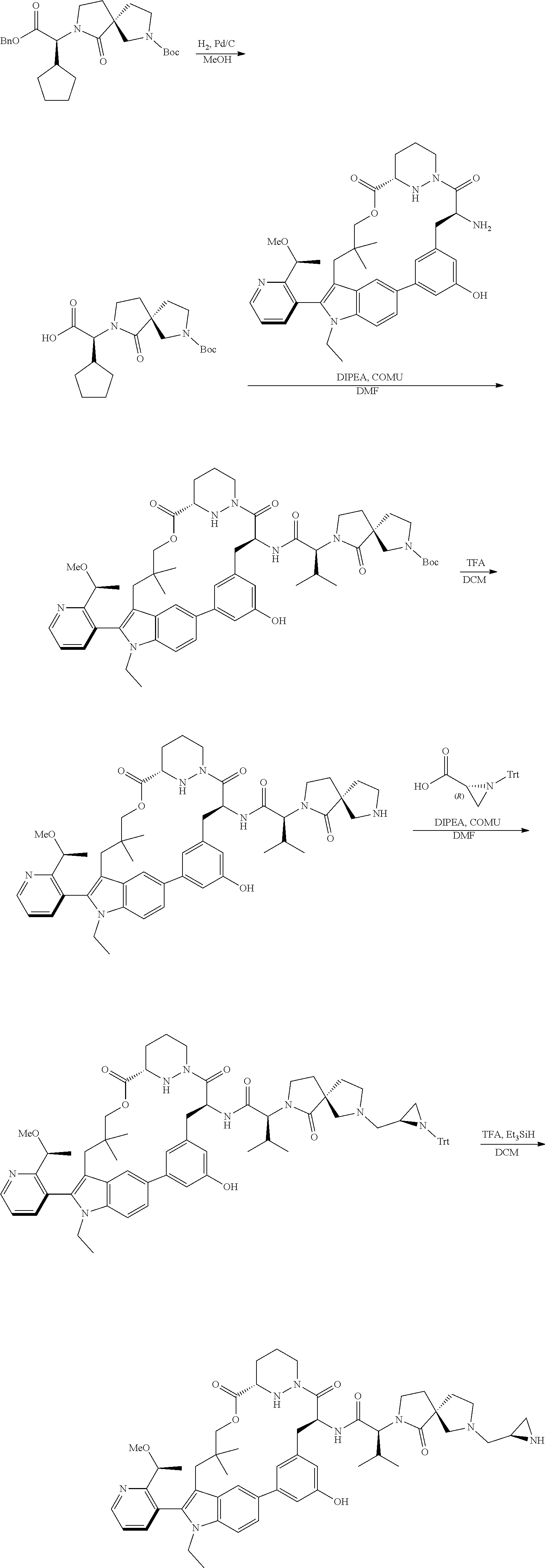

### Step 1: Synthesis of (S)-2-((R)-7-(tert-butoxycarbonyl)-1-oxo-2,7-diazaspiro[4.4]nonan-2-yl)-2-cyclopentylacetic acid

To a solution stirred solution of tert-butyl (R)-7-((S)-2-(benzyloxy)-1-cyclopentyl-2-oxoethyl)-6-oxo-2,7-diazaspiro[4.4]nonane-2-carboxylate (1.0 g, 2.19 mmol) in MeOH (10 mL) at 0° C. was added Pd/C (200 mg). The resulting mixture was stirred for 1 h at room temperature under a hydrogen atmosphere, filtered, and the filter cake washed with MeOH (5×10 mL). The filtrate was concentrated under reduced pressure to afford the desired product (895 mg, crude) which was used without further purification. LCMS (ESI) m/z: [M+H] calcd for C₁₉H₃₀N₂O₅: 376.23; found 367.1.

### Step 2: Synthesis of tert-butyl (5R)-7-((1S)-1-cyclopentyl-2-(((6³S,4S)-1¹-ethyl-2⁵-hydroxy-1²-(2-((5)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-6-oxo-2,7-diazaspiro[4.4]nonane-2-carboxylate

To a stirred solution of (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (702 mg, 1.10 mmol) and DIPEA (1.91 mL, 1.10 mmol) in DMF (500 mL) at 0° C. was added (S)-2-((R)-7-(tert-butoxycarbonyl)-1-oxo-2,7-diazaspiro[4.4]nonan-2-yl)-2-cyclopentylacetic acid (523 mg, 1.43 mmol) and COMU (517 mg, 1.21 mmol). After 1 h at room temperature the reaction mixture was diluted with H₂O (150 mL). The aqueous layer was extracted with EtOAc (3×50 mL) and the combined organic layers were washed with brine, dried with Na₂SO₄, filtered, and concentrated under reduced pressure. Purification by normal phase chromatography (40% EtOAc/pet. ether) afforded the desired product (978 mg, 90.2% yield). LCMS (ESI) m/z: [M+H] calcd for C₅₆H₇₃N₇O₉: 988.56; found 988.7.

### Step 3: Synthesis of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-1-oxo-2,7-diazaspiro[4.4]nonan-2-yl)acetamide

To a stirred solution of tert-butyl (5R)-7-((1S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-6-oxo-2,7-diazaspiro[4.4]nonane-2-carboxylate (300 mg, 0.304 mmol) in DCM (3.0 mL) at 0° C. was added TFA (1.5 mL). The resulting mixture was stirred for 30 min at room temperature. The reaction mixture was then diluted with toluene (2 mL) and concentrated under reduced pressure three times to afford the desired product (270 mg, crude) which was used without further purification. LCMS (ESI) m/z: [M+H] calcd for C₅₁H₆₅N₇O₇: 888.50; found 888.5.

### Step 4: Synthesis of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-1-oxo-7-((R)-1-tritylaziridine-2-carbonyl)-2,7-diazaspiro[4.4]nonan-2-yl)acetamide

To a stirred solution of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-1-oxo-2,7-diazaspiro[4.4]nonan-2-yl)acetamide (270 mg, 0.304 mmol) and DIPEA (0.53 mL, 3.0 mmol) in DMF (3.0 mL) at 0° C. was added (R)-1-tritylaziridine-2-carboxylic acid (130 mg, 0.395 mmol) and COMU (143 mg, 0.334 mmol). After 1 h at room temperature the reaction mixture was diluted with H₂O (30 mL). The aqueous layer was extracted with EtOAc (3×3 mL) and the combined organic layers were washed with brine, dried with Na₂SO₄, filtered, and concentrated under reduced pressure. Purification by prep-TLC (5% MeOH/DCM) afforded the desired product (332 mg, 91.1% yield). LCMS (ESI) m/z: [M+H] calcd for C₇₃H₈₂N₈O₈: 1199.64; found 1199.7.

### Step 5: Synthesis of (2S)-2-((S)-7-((R)-aziridine-2-carbonyl)-1-oxo-2,7-diazaspiro[4.4]nonan-2-yl)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred solution of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-1-oxo-7-((R)-1-tritylaziridine-2-carbonyl)-2,7-diazaspiro[4.4]nonan-2-yl)acetamide (309 mg, 0.258 mmol) in DCM (3.0 mL) at 0° C. was added Et₃SiH (164 mL, 1.03 mmol) and TFA (79 mL, 1.03 mmol). After 30 min the reaction mixture was basified to pH 8 with DIPEA and concentrated under reduced pressure. Purification by reverse phase chromatography (30→60% MeCN/H₂O) afforded the desired product (36 mg, 14.2% yield). LCMS (ESI) m/z: [M+H] calcd for C₅₄H₆₈N₈O₈: 957.53; found 957.3.

### Example 214. Synthesis of (2S)-2-cyclopentyl-2-((S)-7-((2R,3R)-3-cyclopropylaziridine-2-carbonyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

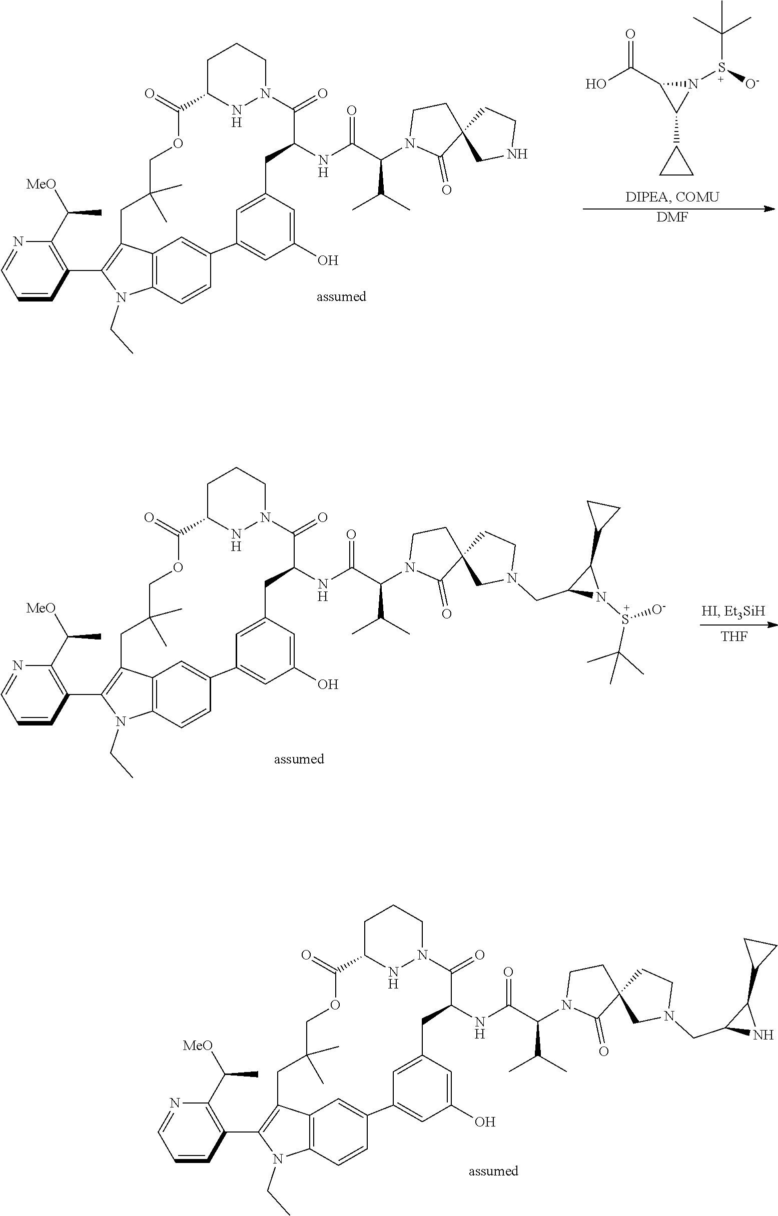

### Step 1: Synthesis of (2S)-2-((5S)-7-((2R,3R)-1-(tert-butylsulfinyl)-3-cyclopropylaziridine-2-carbonyl)-1-oxo-2,7-diazaspiro[4.4]nonan-2-yl)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred solution of (2S)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-1-oxo-2,7-diazaspiro[4.4]nonan-2-yl)acetamide (270 mg, 0.30 mmol) in DMF (3.0 mL) at 0° C. was added DIPEA (530 μL, 3.0 mmol) and (2R,3R)-1-(tert-butylsulfinyl)-3-cyclopropylaziridine-2-carboxylic acid (105 mg, 0.46 mmol) followed by COMU (140 mg, 0.33 mmol). The resulting mixture was stirred for 1 h at room temperature and was then diluted with H₂O (30 mL). The reaction mixture was extracted into EtOAc (3×7 mL). The combined organic layers were washed with brine (3×10 mL), dried over Na₂SO₄, filtered, and concentrated under reduced pressure. Purification by Prep-TLC (6% MeOH/DCM) afforded the desired product (237 mg, 71% yield). LCMS (ESI) m/z: [M+H] calcd for C₆₁H₈₀N₈O₉S: 1101.58; found 1101.3.

### Step 2: Synthesis of (2S)-2-cyclopentyl-2-((S)-7-((2R,3R)-3-cyclopropylaziridine-2-carbonyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)acetamide

To a stirred solution of (2S)-2-((5S)-7-((2R,3R)-1-(tert-butylsulfinyl)-3-cyclopropylaziridine-2-carbonyl)-1-oxo-2,7-diazaspiro[4.4]nonan-2-yl)-2-cyclopentyl-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (230 mg, 0.21 mmol) in THF (2.5 mL) at 0° C. was added Et₃SiH (130 μL, 0.83 mmol) and HI (125 μL, 0.41 mmol, 57% in H₂O). The resulting mixture was stirred for 30 min at room temperature then cooled to 0° C. and neutralized to pH 8. The mixture was concentrated under reduced pressure. Purification by Prep-TLC (8.3% MeOH/DCM) afforded the desired product (46 mg, 21% yield). LCMS (ESI) m/z: [M+H] calcd for C₅₇H₇₂N₈O₈: 997.55; found 997.2.

### Example 209. Synthesis of (2R)—N-(2-(((1S)-1-cyclopentyl-2-(((6³S4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methylaziridine-2-carboxamide

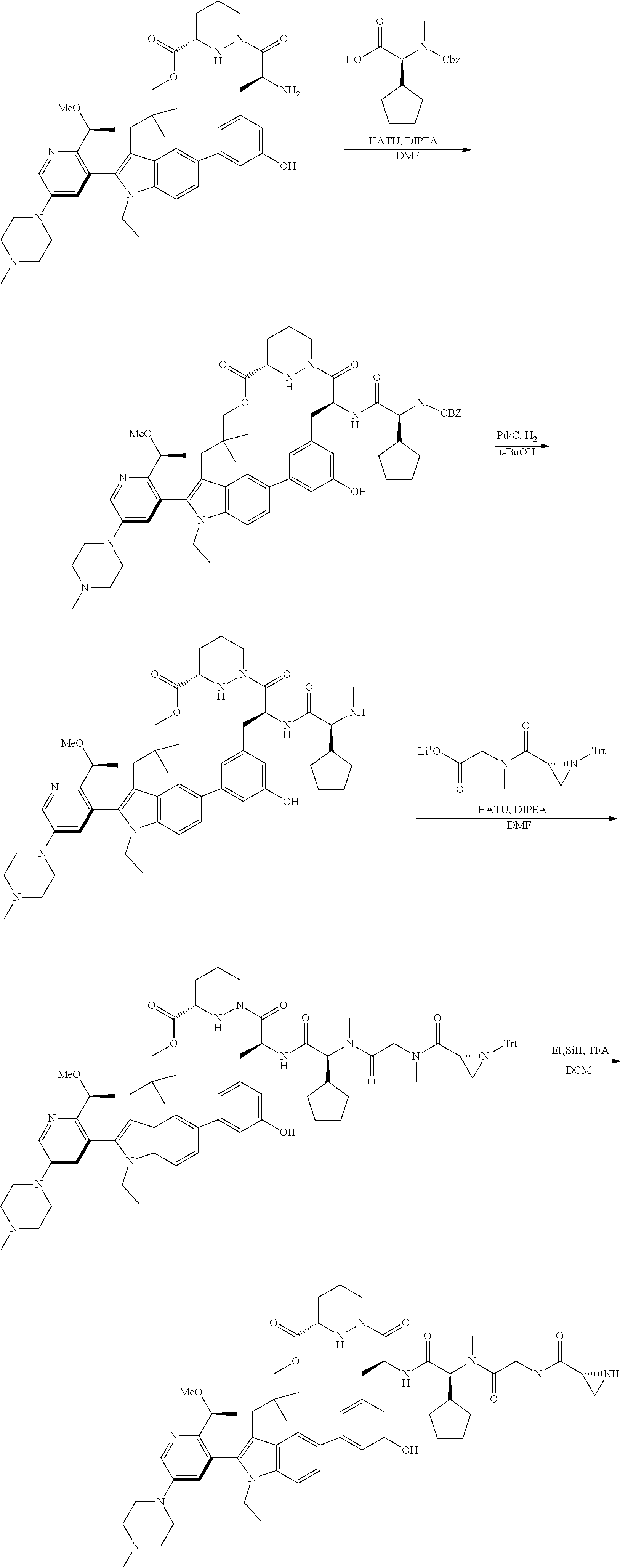

### Step 1: Synthesis of benzyl ((1S)-1-cyclopentyl-2-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamate

To a stirred solution of (6³S, 4S)-4-amino-1¹-ethyl-2⁵-hydroxy-1²-(2-((S)-1-methoxyethyl)-5-(4-methylpiperazin-1-yl)pyridine-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (490 mg, 0.664 mmol) and (S)-2-(((benzyloxy)carbonyl)(methyl)amino)-2-cyclopentylacetic acid (232 mg, 0.797 mmol) in DMF (5 mL) at 0° C. was added DIPEA (1.19 mL, 6.64 mmol) and HATU (303 mg, 0.797 mmol). The resulting mixture was stirred for 1 h at room temperature and then diluted with H₂O (20 mL). The aqueous phase was extracted with EtOAc (3×20 mL) and the combined organic layers were washed with brine, dried over Na₂SO₄, filtered, and concentrated under reduced pressure. Purification by reverse phase chromatography (0→100% MeCN/H₂O, 0.1% NH₄HCO₃) afforded the desired product (420 mg, 59.4% yield). LCMS (ESI) m/z: [M+H] calcd for C₅₈H₇₄N₈O₈: 1011.57; found 1011.6.

### Step 2: Synthesis of (2S)-2-cyclopentyl-N-((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(methylamino)acetamide

To a stirred solution of benzyl ((1S)-1-cyclopentyl-2-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamate (450 mg, 0.445 mmol) in t-BuOH (10 mL) was added Pd/C (90 mg). The resulting mixture was warmed to 40° C. overnight under a hydrogen atmosphere, then filtered and the filter cake washed with MeOH. The filtrate was concentrated under reduced pressure to afford the desired product (420 mg, crude) which was used without further purification. LCMS (ESI) m/z: [M+H] calcd for C₅₀H₆₈N₈O₆: 877.54; found 877.5.

### Step 3: Synthesis of (2R)—N-(2-(((1S)-1-cyclopentyl-2-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methyl-1-tritylaziridine-2-carboxamide

To a stirred solution of (2S)-2-cyclopentyl-N-((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(methylamino)acetamide (130 mg, 0.148 mmol) and lithium (R)—N-methyl-N-(1-tritylaziridine-2-carbonyl)glycinate (78.3 mg, 0.193 mmol) in DMF (2 mL) at 0° C. was added DIPEA (264 mL, 1.48 mmol) and HATU (68 mg, 0.178 mmol). The resulting mixture was stirred for 1 h at room temperature and then diluted with H₂O (20 mL). The aqueous phase was extracted with EtOAc (3×10 mL) and the combined organic layers were washed with H₂O, dried with Na₂SO₄, filtered, and concentrated under reduced pressure. Purification by prep-TLC (10% MeOH/DCM) afforded the desired product (100 mg, 50.9% yield). LCMS (ESI) m/z: [M+Na] calcd for C₇₅H₉₀N₁₀O₈: 1281.69; found 1281.9.

### Step 4: Synthesis of (2R)—N-(2-(((1S)-1-cyclopentyl-2-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methylaziridine-2-carboxamide

To a stirred solution of (2R)—N-(2-(((1S)-1-cyclopentyl-2-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-N-methyl-1-tritylaziridine-2-carboxamide (100 mg, 0.079 mmol) in DCM (1.0 mL) at 0° C. was added Et₃SiH (51 mL, 0.318 mmol) and TFA (24 mL, 0.318 mmol). After 30 min the reaction mixture was basified to pH 8 with DIPEA and concentrated under reduced pressure. Purification by reverse phase chromatography (30→55% MeCN/H₂O) afforded the desired product (14 mg, 16.5% yield). LCMS (ESI) m/z: [M+H] calcd for C₅₆H₇₆N₁₀O₈: 1017.59; found 1017.6.

### Example 268. Synthesis of (2R)—N-(2-(((2S)-1-(((6³S,4S)-1¹-ethyl-2-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(3-(2-oxopyrrolidin-1-yl)propyl)aziridine-2-carboxamide

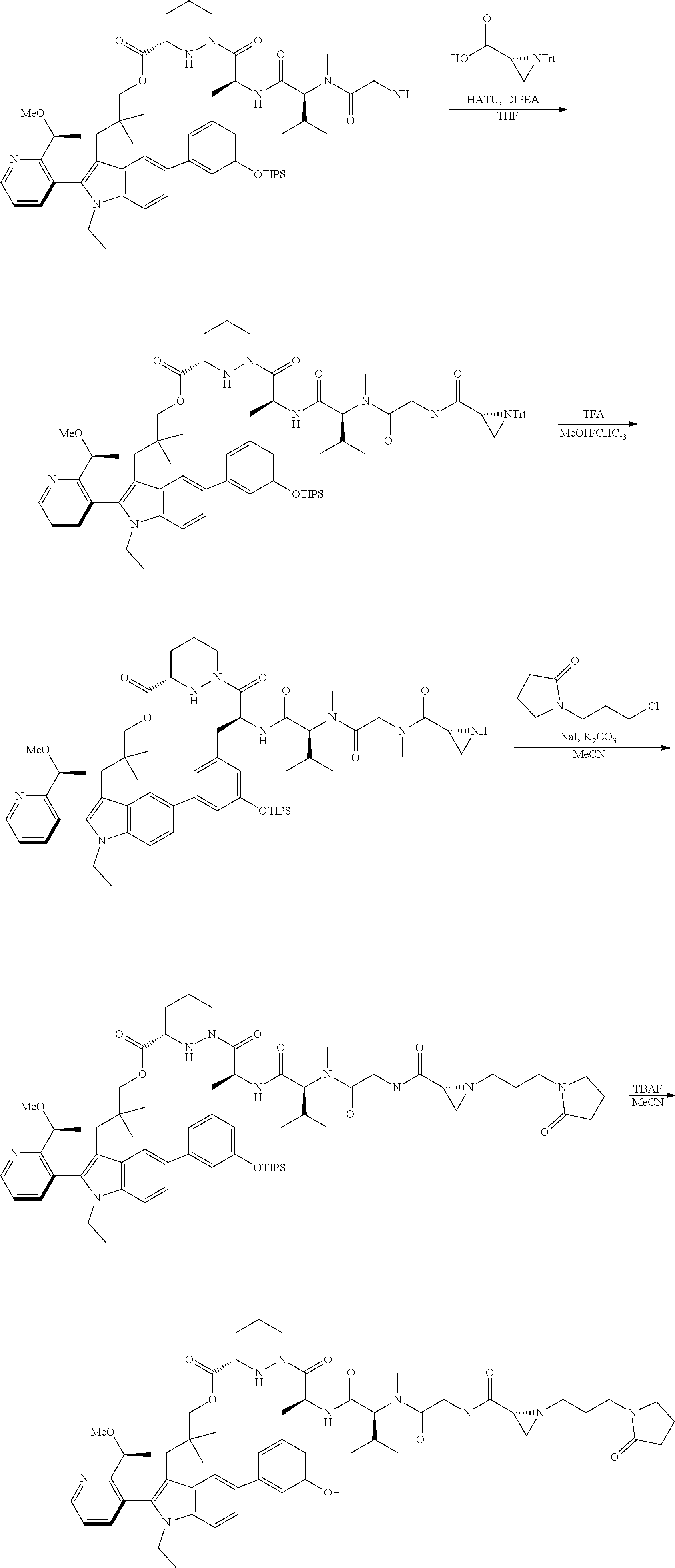

### Step 1: Synthesis of (2R)—N-(2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-tritylaziridine-2-carboxamide

To a solution of (1S)-1-tritylaziridine-2-carboxylic acid (537.6 mg, 1.63 mmol), (2S)—N-((6³S,4S)-1¹-ethyl-2-(2-((S)-1-methoxyethyl)pyridin-3-yl)-10,10-dimethyl-5,7-dioxo-2⁵-((triisopropylsilyl)oxy)-6¹,6²,6³,6⁴,6⁵6⁶-hexahydro-1¹H-8-oxa-1(5,3)-indola-6(1,3)-pyridazina-2(1,3)-benzenacycloundecaphane-. 4-yl)-3-methyl-2-(N-methyl-2-(methylamino)acetamido)butanamide (800 mg, 0.816 mmol) in THF (8 mL) was added DIPEA (0.711 mL, 4.08 mmol), HATU (465.4 mg, 1.22 mmol) at 0° C., the reaction was warmed to room temperature and stirred for 2 h. To the reaction was added H₂O (20 mL), the aqueous phase was extracted with DCM (3×30 mL) and the combined organic phases were washed with brine (20 mL), dried over Na₂SO₄, filtered, and concentrated under reduced pressure. The residue was purified by silica gel column chromatography (0→100% EtOAc/pet. ether) to afford the desired product (1 g, 94.9% yield).

### Step 2: Synthesis of (2R)—N-(2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide

To a solution of (2R)—N-(2-(((2S)-1-(((6³S,4S)-1¹-ethyl-1²-(2-((S)-1-methoxyethyl)pyridine-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-tritylaziridine-2-carboxamide (1 g, 0.774 mmol) in MeOH (5 mL) and CHCl₃ (5 mL) was added TFA (1.15 mL, 15.48 mmol) at 0° C. The reaction was warmed to room temperature and stirred for 2 h. The reaction mixture was added dropwise to aq. NaHCO₃ (30 mL) at 0° C. Then the pH was adjusted to pH 7-8 with using aq. NaHCO₃ at 0° C. The mixture was extracted with DCM (3×20 mL), dried over Na₂SO₄, filtered, and concentrated under reduced pressure to afford the desired product product (960 mg, crude), which was used directly in the next step without further purification.

### Step 3: Synthesis of (2R)—N-(2-(((2S)-1-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(3-(2-oxopyrrolidin-1-yl)propyl)aziridine-2-carboxamide

To a solution of (2R)—N-(2-(((2S)-1-(((6³S,4S)-1¹-ethyl-1²-(2-((S)-1-methoxyethyl)pyridine-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methylaziridine-2-carboxamide (960 mg, 0.915 mmol) in MeCN (10 mL) was added 1-(3-chloropropyl)pyrrolidin-2-one (887.1 mg, 5.49 mmol), K₂CO₃ (1.14 g, 8.23 mmol), NaI (411.4 mg, 2.74 mmol), the reaction was stirred at 80° C. for 24 h. To the reaction was added H₂O (20 mL), the aqueous phase was extracted with EtOAc (3×20 mL). The combined organic phase was dried over Na₂SO₄, filtered, and concentrated under reduced pressure. The residue was purified by reverse phase chromatography (73→93% MeCN/H₂O, 10 mM NH₄HCO₃) to afford product (80 mg, 7.5% yield). LCMS (ESI) m/z: [M+H] calcd for C₆₅H₉₆N₉O₉Si: 1174.7; found 1174.7.

### Step 4: Synthesis of (2R)—N-(2-(((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(3-(2-oxopyrrolidin-1-yl)propyl)aziridine-2-carboxamide

To a solution of (2R)—N-(2-(((2S)-1-(((6³S,4S)-1¹-ethyl-1²-(2-((S)-1-methoxyethyl)pyridine-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-2-oxoethyl)-N-methyl-1-(3-(2-oxopyrrolidin-1-yl)propyl)aziridine-2-carboxamide (80 mg, 0.068 mmol) in THF (1 mL) was added TBAF (1 M, 0.082 mL). The reaction was stirred for 1 h and then was added to H₂O (10 mL), the aqueous phase was extracted with EtOAc (3×10 mL). The combined organic phase was dried over Na₂SO₄, filtered, and concentrated under reduced pressure. The resulting residue was purified by reverse phase chromatography (25→65% MeCN/H₂O, 10 mM NH₄HCO₁ to afford the desired product (42 mg, 60.4% yield). LCMS (ESI) m/z: [M+H] calcd for C₅₆H₇₆N₉O₉: 1018.6; found 1018.5.

The following table of compounds (Table 4) were prepared using the aforementioned methods or variations thereof, as is known to those of skill in the art.

TABLE 4
Exemplary Compounds Prepared by
Methods of the Present Invention

| | Molecular | | Observed MW |
|---|---|---|---|
| Ex# | Formula | Calculated MW | LCMS (ESI) m/z |
| 1 | C₅₀H₆₄N₈O₈ | [M + H] = 905.49 | [M + H] = 905.7 |
| 2 | C₅₀H₆₄N₈O₈ | [M + H] = 905.49 | [M + H] = 905.7 |
| 3 | C₅₁H₆₆N₈O₈ | [M + H] = 919.51 | [M + H] = 919.7 |
| 4 | C₅₁H₆₆N₈O₈ | [M + H] = 919.51 | [M + H] = 919.6 |
| 5 | C₄₉H₆₂N₈O₈ | [M + H] = 891.48 | [M + H] = 891.7 |
| 6 | C₄₉H₆₂N₈O₈ | [M + H] = 891.48 | [M + H] = 891.7 |
| 7 | C₄₈H₆₂N₈O₈ | [M + H] = 879.48 | [M + H] = 879.7 |
| 8 | C₄₈H₆₂N₈O₈ | [M + H] = 879.48 | [M + H] = 879.7 |
| 9 | C₅₆H₆₈N₈O₈ | [M + H] = 969.52 | [M + H] = 969.5 |
| 10 | C₅₆H₆₈N₈O₈ | [M + H] = 969.52 | [M + H] = 969.5 |
| 11 | C₅₄H₆₆N₈O₈ | [M + H] = 955.51 | [M + H] = 955.7 |
| 12 | C₅₄H₆₆N₈O₈ | [M + H] = 955.51 | [M + H] = 955.7 |
| 13 | C₄₉H₆₄N₈O₁₀S | [M + H] = 957.45 | [M + H] = 957.5 |
| 14 | C₅₀H₆₄N₈O₉ | [M + H] = 921.49 | [M + H] = 921.5 |
| 15A | C₅₃H₆₄N₈O₇ | [M + H] = 925.50 | [M + H] = 925.5 |
| 15B | C₅₃H₆₄N₈O₇ | [M + H] = 925.50 | [M + H] = 925.5 |
| 16A | C₅₀H₆₆N₈O₆S | [M + H] = 907.49 | [M + H] = 907.5 |
| 16B | C₅₀H₆₆N₈O₆S | [M + H] = 907.49 | [M + H] = 907.5 |
| 17A | C₅₃H₇₁N₉O₇ | [M + H] = 946.56 | [M + H] = 946.6 |
| 17B | C₅₃H₇₁N₉O₇ | [M + H] = 946.56 | [M + H] = 946.6 |
| 18A | C₅₄H₆₆N₈O₇ | [M + H] = 940.52 | [M + H] = 939.6 |
| 18B | C₅₄H₆₆N₈O₇ | [M + H] = 940.52 | [M + H] = 939.6 |
| 19A | C₅₃H₆₄N₈O₆ | [M + H] = 909.50 | [M + H] = 909.6 |
| 19B | C₅₃H₆₄N₈O₆ | [M + H] = 909.50 | [M + H] = 909.5 |
| 20A | C₅₃H₆₄N₈O₇ | [M + H] = 925.50 | [M + H] = 925.5 |
| 20B | C₅₃H₆₄N₈O₇ | [M + H] = 925.50 | [M + H] = 925.5 |
| 21A | C₄₉H₆₄N₈O₆ | [M + H] = 861.50 | [M + H] = 861.5 |
| 21B | C₄₉H₆₄N₈O₆ | [M + H] = 861.50 | [M + H] = 861.5 |
| 22A | C₄₉H₆₄N₈O₆ | [M + H] = 861.50 | [M + H] = 861.6 |
| 22B | C₄₉H₆₄N₈O₆ | [M + H] = 861.50 | [M + H] = 861.5 |
| 23A | C₄₉H₆₄N₈O₇ | [M + H] = 877.50 | [M + H] = 877.6 |
| 23B | C₄₉H₆₄N₈O₇ | [M + H] = 877.50 | [M + H] = 877.6 |
| 24A | C₅₄H₆₆N₈O₈ | [M + H] = 955.51 | [M + H] = 955.6 |
| 24B | C₅₄H₆₆N₈O₈ | [M + H] = 955.51 | [M + H] = 955.6 |
| 25 | C₄₈H₆₃ClN₈O₈ | [M + H] = 915.45 | [M + H] = 915.7 |
| 26 | C₄₆H₆₀ClN₇O₇ | [M + H] = 858.43 | [M + H] = 858.7 |
| 27 | C₄₈H₆₂N₈O₈ | [M + H] = 879.48 | [M + H] = 879.4 |
| 28 | C₄₇H₆₀N₈O₈ | [M + H] = 865.46 | [M + H] = 865.7 |
| 29 | C₄₅H₅₇N₇O₇ | [M + H] = 808.44 | [M + H] = 808.8 |
| 30 | C₄₆H₅₉N₇O₇ | [M + H] = 822.46 | [M + H] = 822.4 |
| 31 | C₅₀H₆₃N₇O₉ | [M + H] = 906.48 | [M + H] = 906.7 |
| 32A | C₅₄H₆₆N₈O₇ | [M + H] = 940.18 | [M + H] = 939.6 |
| 32B | C₅₄H₆₆N₈O₇ | [M + H] = 940.18 | [M + H] = 939.6 |
| 33 | C₄₈H₆₂N₈O₈ | [M + H] = 879.48 | [M + H] = 878.5 |
| 34 | C₅₀H₆₄N₈O₉ | [M + H] = 921.49 | [M + H] = 921.5 |
| 35 | C₅₀H₆₄N₈O₉ | [M + H] = 921.49 | [M + H] = 921.6 |
| 36 | C₄₉H₆₄N₈O₁₀S | [M + H] = 957.45 | [M + H] = 957.5 |
| 37 | C₄₉H₆₄N₈O₁₀S | [M + H] = 957.45 | [M + H] = 957.5 |
| 38 | C₅₀H₆₄N₈O₁₀ | [M + H] = 937.48 | [M + H] = 937.6 |
| 39 | C₅₀H₆₄N₈O₁₀ | [M + H] = 937.48 | [M + H] = 937.5 |
| 40 | C₄₉H₆₄N₈O₈ | [M + H] = 893.49 | [M + H] = 893.7 |
| 41 | C₄₉H₆₄N₈O₈ | [M + H] = 893.49 | [M + H] = 893.8 |
| 42A | C₄₉H₆₄N₈O₈ | [M + H] = 893.49 | [M + H] = 893.7 |
| 42B | C₄₉H₆₄N₈O₈ | [M + H] = 893.49 | [M + H] = 893.7 |
| 43A | C₅₁H₆₆N₈O₈ | [M + H] = 919.51 | [M + H] = 919.8 |
| 43B | C₅₁H₆₆N₈O₈ | [M + H] = 919.51 | [M + H] = 919.7 |
| 44 | C₅₁H₆₇N₉O₈ | [M + H] = 934.52 | [M + H] = 934.8 |
| 45 | C₅₁H₆₇N₉O₈ | [M + H] = 934.52 | [M + H] = 934.7 |
| 46 | C₅₁H₆₁N₉O₈ | [M + H] = 928.47 | [M + H] = 928.7 |
| 47A | C₅₁H₆₁N₉O₈ | [M + H] = 928.47 | [M + H] = 928.7 |
| 47B | C₅₁H₆₁N₉O₈ | [M + H] = 928.47 | [M + H] = 928.7 |
| 48 | C₅₄H₇₂N₈O₁₁S | [M + H] = 1041.51 | [M + H] = 1041.8 |
| 49 | C₅₀H₆₄N₈O₁₀ | [M + H] = 937.48 | [M + H] = 938.6 |
| 50 | C₅₄H₇₂N₈O₁₁S | [M + H] = 1041.51 | [M + H] = 1041.6 |
| 51 | C₅₀H₆₄N₈O₁₀ | [M + H] = 937.48 | [M + H] = 937.8 |
| 52 | C₅₅H₇₄N₈O₉S | [M + H] = 1023.54 | [M + H] = 1023.8 |
| 53 | C₅₃H₇₂N₈O₉S | [M + Na] = 1019.50 | [M + Na] = 1019.8 |
| 54 | C₅₃H₇₂N₈O₉S | [M + H] = 997.52 | [M + H] = 997.7 |
| 55 | C₅₅H₆₄N₈O₉ | [M + H] = 1003.47 | [M + H] = 1003.8 |
| 56 | C₅₁H₆₈N₈O₉S | [M + Na] = 991.47 | [M + Na] = 991.7 |
| 57 | C₅₁H₆₈N₈O₉S | [M + H] = 969.49 | [M + H] = 969.8 |
| 58 | C₅₃H₆₈N₈O₈ | [M + H] = 945.52 | [M + H] = 945.8 |
| 59 | C₅₂H₆₆N₈O₈ | [M + H] = 931.51 | [M + H] = 931.8 |
| 60 | C₄₆H₅₉N₇O₇ | [M + H] = 822.46 | [M + H] = 822.7 |
| 61A | C₅₅H₆₈N₈O₈ | [M + H] = 969.52 | [M + H] = 969.6 |
| 61B | C₅₅H₆₈N₈O₈ | [M + H] = 969.52 | [M + H] = 969.6 |
| 62A | C₅₅H₆₈N₈O₈ | [M + H] = 955.51 | [M + H] = 955.5 |
| 62B | C₅₅H₆₈N₈O₈ | [M + H] = 955.51 | [M + H] = 955.5 |
| 63 | C₅₄H₆₆N₈O₇ | [M + H] = 939.51 | [M + H] = 939.6 |
| 64A | C₅₅H₆₈N₈O₇ | [M + H] = 953.53 | [M + H] = 953.6 |
| 64B | C₅₅H₆₈N₈O₇ | [M + H] = 953.53 | [M + H] = 953.6 |
| 65A | C₅₅H₆₆N₈O₇ | [M + H] = 951.51 | [M + H] = 951.7 |
| 65B | C₅₅H₆₆N₈O₇ | [M + H] = 951.51 | [M + H] = 951.6 |
| 66 | C₅₄H₆₇N₉O₆ | [M + H] = 938.53 | [M + H] = 938.5 |
| 67 | C₅₆H₆₉N₉O₇ | [M + H] = 980.54 | [M + H] = 980.1 |
| 68A | C₅₂H₆₄N₈O₆S | [M + H] = 929.47 | [M + H] = 929.5 |
| 68B | C₅₂H₆₄N₈O₆S | [M + H] = 929.47 | [M + H] = 929.5 |
| 69A | C₅₁H₆₈N₈O₇ | [M + H] = 891.51 | [M + H] = 891.5 |
| 69B | C₅₁H₆₈N₈O₇ | [M + H] = 891.51 | [M + H] = 891.5 |
| 70A | C₅₆H₆₈N₈O₇ | [M + H] = 965.53 | [M + H] = 965.6 |
| 70B | C₅₆H₆₈N₈O₇ | [M + H] = 965.53 | [M + H] = 965.6 |
| 71A | C₅₆H₆₈N₈O₇ | [M + H] = 965.53 | [M + H] = 965.5 |
| 71B | C₅₆H₆₈N₈O₇ | [M + H] = 965.53 | [M + H] = 965.5 |
| 72A | C₅₁H₆₆N₈O₇ | [M + H] = 903.51 | [M + H] = 903.5 |
| 72B | C₅₁H₆₆N₈O₇ | [M + H] = 903.51 | [M + H] = 903.6 |
| 73 | C₅₅H₆₈N₈O₇ | [M + H] = 953.53 | [M + H] = 953.6 |
| 74 | C₅₀H₆₆N₈O₇ | [M + H] = 891.51 | [M + H] = 891.6 |
| 75 | C₅₅H₆₈N₈O₈ | [M + H] = 969.52 | [M + H] = 969.9 |
| 76 | C₅₈H₇₂N₈O₇ | [M + H] = 993.56 | [M + H] = 993.5 |
| 77 | C₅₇H₇₀N₈O₇ | [M + H] = 979.54 | [M + H] = 979.5 |
| 78 | C₅₂H₆₈N₈O₇ | [M + H] = 917.53 | [M + H] = 917.5 |
| 79A | C₅₀H₆₆N₈O₆ | [M + H] = 875.52 | [M + H] = 875.6 |
| 79B | C₅₀H₆₆N₈O₆ | [M + H] = 875.52 | [M + H] = 875.6 |
| 80 | C₅₀H₆₆N₈O₆ | [M + H] = 875.52 | [M + H] = 875.6 |
| 81 | C₅₄H₆₆N₈O₆ | [M + H] = 923.52 | [M + H] = 923.5 |
| 82A | C₅₄H₆₆N₈O₆ | [M + H] = 923.52 | [M + H] = 923.6 |
| 82B | C₅₄H₆₆N₈O₆ | [M + H] = 923.52 | [M + H] = 923.6 |
| 83 | C₅₁H₆₄N₁₀O₈ | [M + H] = 945.50 | [M + H] = 945.6 |
| 84 | C₅₁H₆₄N₁₀O₈ | [M + H] = 945.50 | [M + H] = 945.6 |
| 85 | C₅₁H₆₈N₈O₉ | [M + H] = 937.52 | [M + H] = 837.6 |
| 86 | C₅₂H₆₈N₈O₈ | [M + H] = 933.52 | [M + H] = 933.7 |
| 87 | C₅₁H₆₂N₈O₇ | [M + H] = 899.48 | [M + H] = 899.5 |
| 88 | C₅₂H₆₈N₈O₈ | [M + H] = 933.52 | [M + H] = 933.6 |
| 89 | C₅₂H₆₈N₈O₈ | [M + H] = 933.52 | [M + H] = 933.6 |
| 90 | C₅₂H₇₀N₈O₉ | [M + H] = 951.53 | [M + H] = 951.6 |
| 91 | C₄₉H₆₆N₈O₉S | [M + H] = 943.48 | [M + H] = 943.6 |
| 92 | C₄₉H₆₆N₈O₉S | [M + H] = 943.48 | [M + H] = 943.5 |
| 93 | C₅₅H₆₈N₈O₉ | [M + H] = 985.52 | [M + H] = 985.7 |
| 94 | C₅₂H₆₆N₈O₁₀ | [M + H] = 963.50 | [M + H] = 963.6 |
| 95 | C₅₁H₆₆N₈O₈ | [M + H] = 919.51 | [M + H] = 919.5 |
| 96 | C₅₅H₆₈N₈O₉ | [M + H] = 985.52 | [M + H] = 985.7 |
| 97 | C₄₈H₆₈N₈O₉ | [M + H] = 985.52 | [M + H] = 985.7 |
| 98 | C₄₈H₅₈N₈O₉ | [M + H] = 891.44 | [M + H] = 891.6 |
| 99 | C₄₆H₅₉N₇O₇ | [M + H] = 822.46 | [M + H] = 822.6 |
| 100 | C₄₆H₅₉N₇O₇ | [M + H] = 822.46 | [M + H] = 822.6 |
| 101 | C₅₅H₇₄N₈O₉S | [M + Na] = 1045.52 | [M + Na] = 1045.7 |
| 102 | C₅₀H₆₆N₈O₈ | [M + H] = 907.51 | [M + H] = 907.7 |
| 103 | C₅₁H₆₆N₈O₈ | [M + H] = 919.51 | [M + H] = 919.5 |
| 104 | C₅₁H₆₆N₈O₈ | [M + H] = 919.51 | [M + H] = 919.7 |
| 105 | C₄₉H₆₄N₈O₈ | [M + H] = 893.49 | [M + H] = 893.7 |
| 106 | C₅₀H₆₆N₈O₇ | [M + H] = 903.51 | [M + H] = 930.7 |
| 107 | C₄₉H₆₄N₈O₇ | [M + H] = 877.50 | [M + H] = 877.7 |
| 108 | C₅₂H₇₀N₈O₉S | [M + H] = 983.51 | [M + H] = 983.8 |
| 109 | C₅₁H₆₄N₈O₈ | [M + H] = 917.49 | [M + H] = 917.7 |
| 110 | C₅₄H₆₆N₈O₈ | [M + H] = 955.51 | [M + H] = 955.7 |
| 111 | C₅₂H₆₆N₈O₁₀ | [M + H] = 963.50 | [M + H] = 963.8 |
| 112 | C₄₆H₅₉N₇O₇ | [M + H] = 822.46 | [M + H] = 822.6 |
| 113 | C₅₂H₆₆N₈O₈ | [M + Na] = 953.49 | [M + Na] = 953.6 |
| 114 | C₅₀H₆₆N₈O₈ | [M + H] = 907.51 | [M + H] = 907.7 |
| 115 | C₅₁H₆₄N₈O₈ | [M + H] = 917.49 | [M + H] = 917.6 |
| 116 | C₅₄H₆₆N₈O₈ | [M + H] = 955.51 | [M + H] = 955.7 |
| 117 | C₅₀H₆₆N₈O₉S | [M + H] = 955.48 | [M + H] = 955.6 |
| 118 | C₅₀H₆₆N₈O₉S | [M + H] = 955.48 | [M + H] = 955.8 |
| 119 | C₅₂H₇₀N₈O₉S | [M + H] = 983.51 | [M + H] = 983.7 |
| 120 | C₄₈H₆₂N₈O₈ | [M + H] = 879.48 | [M + H] = 880.2 |
| 121 | C₅₁H₆₈N₈O₆ | [M + H] = 889.53 | [M + H] = 889.6 |
| 122 | C₅₁H₆₈N₈O₆ | [M + H] = 889.53 | [M + H] = 889.6 |
| 123 | C₅₅H₇₀N₈O₆ | [M + H] = 939.55 | [M + H] = 939.6 |
| 124 | C₅₃H₇₀N₈O₆ | [M + H] = 915.55 | [M + H] = 915.6 |
| 125 | C₅₄H₇₂N₈O₆ | [M + H] = 929.57 | [M + H] = 929.6 |
| 126 | C₅₇H₇₀N₈O₆ | [M + H] = 963.55 | [M + H] = 963.6 |
| 127 | C₅₂H₆₃N₉O₈ | [M + H] = 942.49 | [M + H] = 942.4 |
| 128 | C₅₅H₇₀N₈O₈ | [M + H] = 971.54 | [M + H] = 971.5 |
| 129 | C₄₉H₆₂N₈O₈ | [M + H] = 891.48 | [M + H] = 891.4 |
| 130 | C₄₉H₆₂N₈O₈ | [M + H] = 891.48 | [M + H] = 891.5 |
| 131 | C₅₂H₆₃N₇O₇ | [M + H] = 898.49 | [M + H] = 898.4 |
| 132 | C₅₂H₆₃N₇O₇ | [M + H] = 910.46 | [M + H] = 910.4 |
| 133 | C₅₂H₆₈N₈O₈ | [M + H] = 933.52 | [M + H] = 933.6 |
| 134 | C₅₂H₆₈N₈O₈ | [M + H] = 919.51 | [M + H] = 919.5 |
| 135 | C₅₂H₆₈N₈O₈ | [M + H] = 933.52 | [M + H] = 933.5 |
| 136 | C₅₂H₆₈N₈O₈ | [M + H] = 933.52 | [M + H] = 933.6 |
| 137 | C₅₃H₇₂N₈O₉ | [M + H] = 965.55 | [M + H] = 966.2 |
| 138 | C₅₂H₆₈N₈O₇ | [M + H] = 917.53 | [M + H] = 918.2 |
| 139 | C₅₂H₆₈N₈O₇ | [M + H] = 917.53 | [M + H] = 917.4 |
| 140 | C₄₈H₆₂N₈O₈ | [M + H] = 879.48 | [M + H] = 879.4 |
| 141 | C₅₁H₆₆N₈O₈ | [M + H] = 919.51 | [M + H] = 919.1 |
| 142 | C₅₂H₇₀N₈O₈ | [M + H] = 935.54 | [M + H] = 936.3 |
| 143 | C₅₁H₆₅N₉O₉ | [M + H] = 965.52 | [M + H] = 966.5 |
| 144 | C₅₂H₆₉N₉O₈ | [M + H] = 948.53 | [M + H] = 948.5 |
| 145 | C₅₂H₆₃N₇O₇ | [M + H] = 898.49 | [M + H] = 898.5 |
| 146 | C₅₂H₆₆N₈O₈ | [M + H] = 931.51 | [M + H] = 931.5 |
| 147 | C₄₈H₆₂N₈O₈ | [M + H] = 879.48 | [M + H] = 879.5 |
| 148 | C₅₂H₇₀N₈O₈ | [M + H] = 934.53 | [M + H] = 936.3 |
| 149 | C₅₂H₆₆N₈O₈ | [M + H] = 931.51 | [M + H] = 931.5 |
| 150 | C₄₉H₆₄N₈O₈ | [M + H] = 893.49 | [M + H] = 893.5 |
| 151 | C₅₁H₆₆N₈O₈ | [M + H] = 919.51 | [M + H] = 919.5 |
| 152 | C₅₁H₆₆N₈O₈ | [M + H] = 919.51 | [M + H] = 919.5 |
| 153 | C₅₂H₆₈N₈O₈ | [M + H] = 933.52 | [M + H] = 934.5 |
| 154 | C₅₂H₆₈N₈O₈ | [M + H] = 933.52 | [M + H] = 934.5 |
| 155 | C₅₆H₆₉FN₈O₈ | [M + H] = 1001.53 | [M + H] = 1001.9 |
| 156 | C₅₇H₇₂N₈O₉ | [M + H] = 1013.55 | [M + H] = 1013.9 |
| 157 | C₅₀H₆₄N₈O₈ | [M + H] = 905.49 | [M + H] = 905.5 |
| 158 | C₅₀H₆₄N₈O₈ | [M + H] = 905.49 | [M + H] = 905.6 |
| 159 | C₅₂H₇₀N₈O₉ | [M + H] = 951.53 | [M + H] = 951.6 |
| 160 | C₅₂H₇₀N₈O₉ | [M + H] = 951.53 | [M + H] = 951.6 |
| 161 | C₅₁H₆₈N₈O₉ | [M + H] = 937.52 | [M + H] = 937.6 |
| 162 | C₅₁H₆₈N₈O₉ | [M + H] = 937.52 | [M + H] = 937.6 |
| 163 | C₅₂H₆₆N₈O₈ | [M + H] = 931.51 | [M + H] = 931.5 |
| 164 | C₅₂H₆₆N₈O₈ | [M + H] = 931.51 | [M + H] = 931.5 |
| 165 | C₅₂H₆₈N₈O₈ | [M + H] = 933.53 | [M + H] = 933.5 |
| 166 | C₅₂H₆₆N₈O₈ | [M + H] = 931.51 | [M + H] = 931.5 |
| 167 | C₅₁H₆₆N₈O₈ | [M + H] = 919.51 | [M + H] = 919.7 |
| 168 | C₄₈H₆₄N₁₀O₇S | [M + H] = 925.48 | [M + H] = 925.30 |
| 169 | C₄₈H₆₄N₁₀O₇S | [M + H] = 925.48 | [M + H] = 925.30 |
| 170 | C₅₄H₇₀N₈O₈ | [M + H] = 959.54 | [M + H] = 959.40 |
| 171 | C₅₁H₆₅FN₈O₈ | [M + H] = 937.50 | [M + H] = 937.3 |
| 172 | C₅₂H₆₆N₈O₈ | [M + H] = 919.51 | [M + H] = 919.3 |
| 173 | C₅₂H₆₉N₉O₈ | [M + H] = 948.54 | [M + H] = 948.5 |
| 174 | C₅₄H₆₈N₈O₈ | [M + H] = 957.53 | [M + H] = 957.3 |
| 175 | C₅₄H₆₈N₈O₈ | [M + H] = 957.53 | [M + H] = 957.3 |
| 176 | C₅₂H₆₈N₈O₇ | [M + H] = 917.53 | [M + H] = 918.1 |
| 177 | C₅₁H₆₇N₉O₈ | [M + H] = 934.52 | [M + H] = 934.3 |
| 178 | C₅₁H₆₆N₈O₈ | [M − H] = 917.49 | [M − H] = 917.6 |
| 179 | C₅₁H₆₆N₈O₈ | [M − H] = 917.49 | [M − H] = 917.7 |
| 180 | C₅₃H₆₈N₈O₈ | [M + H] = 945.53 | [M + H] = 945.6 |
| 181 | C₅₃H₆₈N₈O₈ | [M + H] = 943.51 | [M + H] = 943.6 |
| 182 | C₅₁H₆₆N₈O₈ | [M + H] = 919.51 | [M + H] = 919.8 |
| 183 | C₅₁H₆₅N₉O₉ | [M + H] = 918.50 | [M + H] = 948.6 |
| 184 | C₅₁H₆₅FN₈O₈ | [M + H] = 937.50 | [M + H] = 937.3 |
| 185 | C₅₁H₆₅FN₈O₈ | [M + H] = 937.50 | [M + H] = 937.2 |
| 186 | C₅₁H₆₆N₈O₈ | [M + H] = 919.51 | [M + H] = 919.5 |
| 187 | C₅₂H₆₃N₉O₈ | [M + H] = 942.49 | [M + H] = 942.3 |
| 188 | C₅₀H₆₅N₉O₈ | [M + H] = 920.51 | [M + H] = 920.3 |
| 189 | C₅₀H₆₅N₉O₈ | [M + H] = 920.51 | [M + H] = 920.3 |
| 190 | C₅₂H₆₉N₉O₈ | [M + H] = 948.54 | [M + H] = 948.5 |
| 191 | C₅₃H₆₈N₈O₈ | [M + H] = 945.53 | [M + H] = 945.5 |
| 192 | C₅₃H₆₈N₈O₈ | [M + H] = 945.53 | [M + H] = 945.4 |
| 193 | C₅₂H₆₈N₈O₇ | [M + H] = 917.53 | [M + H] = 918.0 |
| 194 | C₅₁H₆₅N₉O₉ | [M + H₂O] = 965.50 | [M + H] = 966.4 |
| 195 | C₅₃H₆₈N₈O₈ | [M + H] = 945.53 | [M + H] = 945.5 |
| 196 | C₅₃H₆₈N₈O₈ | [M + H] = 945.53 | [M + H] = 945.4 |
| 197 | C₅₁H₆₇N₉O₈ | [M + H] = 934.52 | [M + H] = 934.5 |
| 198 | C₅₄H₇₀N₈O₈ | [M + H] = 959.54 | [M + H] = 959.3 |
| 199 | C₅₃H₆₈N₈O₈ | [M + H] = 945.53 | [M + H] = 945.4 |
| 200 | C₅₃H₆₈N₈O₈ | [M + H] = 945.53 | [M + H] = 945.5 |
| 201 | C₅₃H₇₂N₈O₉ | [M + H] = 965.55 | [M + H] = 965.4 |
| 202 | C₅₃H₆₈N₈O₈ | [M + H] = 945.53 | [M + H] = 945.8 |
| 203 | C₅₁H₆₇N₉O₈ | [M + H] = 934.52 | [M + H] = 934.8 |
| 204 | C₅₁H₆₉N₉O₈ | [M + H] = 936.54 | [M + H] = 936.8 |
| 205 | C₅₁H₆₅N₉O₉ | [M + H] = 948.50 | [M + H] = 948.5 |
| 206 | C₅₂H₆₈N₈O₈ | [M + H] = 933.53 | [M + H] = 933.7 |
| 207 | C₅₂H₆₈N₈O₈ | [M + H] = 933.53 | [M + H] = 933.5 |
| 208 | C₅₂H₆₈N₈O₈ | [M + H] = 933.53 | [M + H] = 933.7 |
| 209 | C₅₆H₇₆N₁₀O₈ | [M + H] = 1017.59 | [M + H] = 1017.6 |
| 210 | C₅₁H₆₇N₉O₇S | [M + H] = 950.50 | [M + H] = 950.2 |
| 211 | C₅₁H₆₇N₇O₈ | [M + H] = 906.52 | [M + H] = 906.4 |
| 212 | C₅₀H₆₁N₉O₈S | [M + H] = 948.45 | [M + H] = 948.1 |
| 213 | C₄₉H₆₂N₈O₈ | [M + H] = 891.48 | [M + H] = 891.4 |
| 214 | C₅₇H₇₂N₈O₈ | [M + H] = 997.56 | [M + H] = 997.2 |
| 215 | C₅₇H₇₂N₈O₈ | [M + H] = 997.56 | [M + H] = 997.2 |
| 216 | C₄₈H₆₆N₁₀O₇S | [M + H] = 927.49 | [M + H] = 927.5 |
| 217 | C₄₈H₆₆N₁₀O₇S | [M + H] = 927.49 | [M + H] = 927.4 |
| 218 | C₄₈H₆₄N₁₀O₇S | [M + H] = 925.48 | [M + H] = 925.4 |
| 219 | C₄₈H₆₄N₁₀O₇S | [M + H] = 925.48 | [M + H] = 925.1 |
| 220 | C₄₈H₆₄N₁₀O₇S | [M + H] = 925.48 | [M + H] = 925.4 |
| 221 | C₄₈H₆₄N₁₀O₇S | [M + H] = 925.48 | [M + H] = 925.4 |
| 222 | C₄₉H₆₆N₁₀O₇S | [M + H] = 939.49 | [M + H] = 939.5 |
| 223 | C₄₉H₆₆N₁₀O₇S | [M + H] = 939.49 | [M + H] = 939.5 |
| 224 | C₄₉H₆₆N₁₀O₇S | [M + H] = 939.49 | [M + H] = 940.0 |
| 225 | C₄₉H₆₆N₁₀O₇S | [M + H] = 939.49 | [M + H] = 939.2 |
| 226 | C₄₉H₆₆N₁₀O₇S | [M + H] = 939.49 | [M + H] = 939.1 |
| 227 | C₄₉H₆₆N₁₀O₇S | [M + H] = 939.49 | [M + H] = 939.9 |
| 228 | C₄₉H₆₆N₁₀O₇S | [M + H] = 939.49 | [M + H] = 940.0 |
| 229 | C₄₉H₆₆N₁₀O₇S | [M + H] = 939.49 | [M + H] = 939.1 |
| 230 | C₅₇H₇₇N₉O₈ | [M + H] = 1016.60 | [M + H] = 1016.6 |
| 231 | C₅₁H₆₄N₈O₉ | [M + H] = 933.49 | [M + H] = 933.1 |
| 232 | C₅₁H₆₄N₈O₉ | [M + H] = 933.49 | [M + H] = 933.2 |
| 233 | C₅₅H₆₈F₂N₈O₇ | [M + H] = 991.53 | [M + H] = 992.0 |
| 234 | C₅₄H₆₉N₉O₇S | [M + H] = 988.51 | [M + H] = 988.1 |
| 235 | C₄₉H₆₆N₁₀O₇S | [M + H] = 939.49 | [M + H] = 939.5 |
| 236 | C₅₂H₆₈N₈O₈ | [M + H] = 933.53 | [M + H] = 933.5 |
| 237 | C₅₂H₆₈N₈O₈ | [M + H] = 933.53 | [M + H] = 933.5 |
| 238 | C₅₂H₆₈N₈O₈ | [M + H] = 933.53 | [M + H] = 933.5 |
| 239 | C₅₂H₆₈N₈O₈ | [M + H] = 933.53 | [M + H] = 933.5 |
| 240 | C₅₂H₆₈N₈O₈ | [M + H] = 933.53 | [M + H] = 933.5 |
| 241 | C₅₂H₆₈N₈O₈ | [M + H] = 933.53 | [M + H] = 933.5 |
| 242 | C₅₂H₆₈N₈O₈ | [M + H] = 933.53 | [M + H] = 933.5 |
| 243 | C₅₂H₆₈N₈O₈ | [M + H] = 933.53 | [M + H] = 933.5 |
| 244 | C₅₂H₆₆N₈O₈ | [M + H] = 931.51 | [M + H] = 931.5 |
| 245 | C₅₄H₇₀N₈O₈ | [M + H] = 959.54 | [M + H] = 959.5 |
| 246 | C₅₄H₇₀N₈O₈ | [M + H] = 959.54 | [M + H] = 959.5 |
| 247 | C₅₃H₇₀N₈O₈ | [M + H] = 947.54 | [M + H] = 947.4 |
| 248 | C₅₂H₆₈N₈O₉ | [M + H] = 949.52 | [M + H] = 949.5 |
| 249 | C₅₂H₆₈N₈O₉ | [M + H] = 949.52 | [M + H] = 949.5 |
| 250 | C₅₂H₆₇N₉O₈ | [M + H] = 946.52 | [M + H] = 946.5 |
| 251 | C₅₂H₆₇N₉O₈ | [M + H] = 946.52 | [M + H] = 946.5 |
| 252 | C₅₂H₆₉N₉O₈ | [M + H] = 948.54 | [M + H] = 948.5 |
| 253 | C₅₂H₆₉N₉O₈ | [M + H] = 948.54 | [M + H] = 948.5 |
| 254 | C₅₂H₆₉N₉O₈ | [M + H] = 948.54 | [M + H] = 948.5 |
| 255 | C₅₂H₆₉N₉O₈ | [M + H] = 948.54 | [M + H] = 948.5 |
| 256 | C₅₀H₆₆N₉O₈ | [M + H] = 907.51 | [M + H] = 907.5 |
| 257 | C₅₀H₆₆N₈O₈ | [M + H] = 907.51 | [M + H] = 907.5 |
| 258 | C₅₀H₆₃F₃N₈O₈ | [M + H] = 961.48 | [M + H] = 961.5 |
| 259 | C₅₀H₆₃F₃N₈O₈ | [M + H] = 961.48 | [M + H] = 961.5 |
| 260 | C₅₀H₆₃F₃N₈O₈ | [M + H] = 961.48 | [M + H] = 961.5 |
| 261 | C₅₀H₆₃F₃N₈O₈ | [M + H] = 961.48 | [M + H] = 961.5 |
| 262 | C₅₁H₆₈N₈O₈ | [M + H] = 921.53 | [M + H] = 921.5 |
| 263 | C₅₁H₆₈N₈O₈ | [M + H] = 921.53 | [M + H] = 921.5 |
| 264 | C₅₂H₇₀N₈O₈ | [M + H] = 935.54 | [M + H] = 935.3 |
| 265 | C₅₂H₇₀N₈O₈ | [M + H] = 935.54 | [M + H] = 935.3 |
| 266 | C₅₄H₇₂N₈O₁₀ | [M + H] = 993.55 | [M + H] = 993.5 |
| 267 | C₅₄H₇₂N₈O₁₀ | [M + H] = 993.55 | [M + H] = 993.5 |
| 268 | C₅₆H₇₅N₉O₉ | [M + H] = 1018.58 | [M + H] = 1018.6 |
| 269 | C₅₆H₇₅N₉O₉ | [M + H] = 1018.58 | [M + H] = 1018.5 |
| 270 | C₅₃H₇₁N₉O₉ | [M + H] = 978.55 | [M + H] = 978.5 |
| 271 | C₅₃H₇₁N₉O₉ | [M + H] = 978.55 | [M + H] = 978.5 |
| 272 | C₅₅H₇₃N₉O₁₀ | [M + H] = 1020.56 | [M + H] = 1020.5 |
| 273 | C₅₃H₆₉N₉O₈ | [M + H] = 960.54 | [M + H] = 960.5 |
| 274 | C₅₃H₆₉N₉O₈ | [M + H] = 960.54 | [M + H] = 960.5 |
| 275 | C₅₂H₆₈N₈O₉ | [M + H] = 949.52 | [M + H] = 949.3 |
| 276 | C₅₂H₆₈N₈O₉ | [M + H] = 949.52 | [M + H] = 949.3 |
| 277 | C₅₃H₇₀N₈O₉ | [M + H] = 963.54 | [M + H] = 963.5 |
| 278 | C₅₃H₇₀N₈O₉ | [M + H] = 963.54 | [M + H] = 963.5 |
| 279 | C₅₄H₇₂N₈O₉ | [M + H] = 977.55 | [M + H] = 977.5 |
| 280 | C₅₄H₇₂N₈O₉ | [M + H] = 977.55 | [M + H] = 977.6 |
| 281 | C₅₂H₆₈N₈O₉ | [M + H] = 949.52 | [M + H] = 949.3 |
| 282 | C₅₂H₆₈N₈O₉ | [M + H] = 949.52 | [M + H] = 949.3 |
| 283 | C₅₂H₆₈N₈O₉ | [M + H] = 949.52 | [M + H] = 949.5 |
| 284 | C₅₂H₆₈N₈O₉ | [M + H] = 949.52 | [M + H] = 949.5 |
| 285 | C₅₂H₆₈N₈O₈ | [M + H] = 933.53 | [M + H] = 933.5 |
| 286 | C₄₉H₆₃N₇O₇ | [M + H] = 862.49 | [M + H] = 862.4 |
| 287 | C₅₃H₇₀N₈O₈ | [M + H] = 947.54 | [M + H] = 947.5 |
| 288 | C₅₃H₇₀N₈O₈ | [M + H] = 947.54 | [M + H] = 947.5 |
| 289 | C₅₀H₆₈N₁₀O₇S | [M + H] = 953.51 | [M + H] = 953.5 |
| 290 | C₅₀H₆₈N₁₀O₇S | [M + H] = 953.51 | [M + H] = 953.4 |
| 291 | C₅₀H₆₈N₁₀O₇S | [M + H] = 953.51 | [M + H] = 953.5 |
| 292 | C₅₀H₆₈N₁₀O₇S | [M + H] = 953.51 | [M + H] = 953.5 |
| 293 | C₄₉H₆₆N₁₀O₇S | [M + H] = 939.49 | [M + H] = 939.5 |
| 294 | C₄₉H₆₆N₁₀O₇S | [M + H] = 939.49 | [M + H] = 939.5 |
| 295 | C₅₀H₆₈N₁₀O₇S | [M + H] = 953.51 | [M + H] = 953.5 |
| 296 | C₄₉H₆₆N₁₀O₇S | [M + H] = 939.49 | [M + H] = 939.4 |
| 297 | C₄₉H₆₆N₁₀O₇S | [M + H] = 939.49 | [M + H] = 939.5 |
| 298 | C₄₉H₆₆N₁₀O₇S | [M + H] = 939.49 | [M + H] = 939.5 |
| 299 | C₄₉H₆₆N₁₀O₇S | [M + H] = 939.49 | [M + H] = 939.5 |
| 300 | C₄₉H₆₆N₁₀O₇S | [M + H] = 939.49 | [M + H] = 939.4 |
| 301 | C₄₉H₆₆N₁₀O₇S | [M + H] = 939.49 | [M + H] = 939.4 |
| 302 | C₄₉H₆₆N₁₀O₇S | [M + H] = 939.49 | [M + H] = 939.4 |
| 303 | C₅₀H₆₆N₁₀O₇S | [M + H] = 951.49 | [M + H] = 951.5 |
| 304 | C₅₀H₆₆N₁₀O₇S | [M + H] = 951.49 | [M + H] = 951.5 |
| 305 | C₅₅H₇₀N₈O₈ | [M + H] = 971.54 | [M + H] = 971.1 |
| 306 | C₅₂H₆₆N₈O₈ | [M + H] = 931.51 | [M + H] = 931.2 |
| 307 | C₅₂H₆₆N₈O₈ | [M + H] = 931.51 | [M + H] = 931.2 |
| 308 | C₅₅H₆₉N₉O₈ | [M + H] = 984.54 | [M + H] = 984.5 |
| 309 | C₅₃H₇₀N₈O₈ | [M + H] = 947.54 | [M + H] = 947.5 |
| 310 | C₄₉H₆₆N₁₀O₇S | [M + H] = 939.49 | [M + H] = 939.5 |
| 311 | C₅₁H₆₅FN₈O₈ | [M + H] = 937.50 | [M + H] = 937.2 |
| 312 | C₄₉H₆₃N₇O₈ | [M + H] = 878.48 | [M + H] = 878.4 |
| 313 | C₅₇H₇₂N₈O₇ | [M + H] = 981.56 | [M + H] = 981.5 |
| 314 | C₅₄H₆₉FN₈O₈ | [M + H] = 977.53 | [M + H] = 977.5 |
| 315 | C₅₄H₆₉FN₈O₈ | [M + H] = 977.53 | [M + H] = 977.5 |
| 316 | C₅₅H₆₉N₉O₈ | [M + H] = 984.54 | [M + H] = 984.5 |
| 317 | C₅₈H₇₈N₈O₉ | [M + H] = 1031.60 | [M + H] = 1031.5 |
| 318 | C₅₈H₇₂F₂N₈O₇ | [M + H] = 1031.56 | [M + H] = 1031.5 |
| 319 | C₅₈H₇₂F₂N₈O₇ | [M + H] = 948.48 | [M + H] = 948.4 |
| 320 | C₅₁H₆₅N₉O₇S | [M + H] = 993.54 | [M + H] = 993.5 |
| 321 | C₅₅H₇₀F₂N₈O₇ | [M + H] = 1013.57 | [M + H] = 1013.5 |
| 322 | C₅₅H₇₀F₂N₈O₇ | [M + H] = 989.55 | [M + H] = 989.5 |
| 323 | C₅₈H₇₃FN₈O₇ | [M + H] = 989.55 | [M + H] = 989.5 |
| 324 | C₅₈H₇₃FN₈O₇ | [M + H] = 1086.60 | [M + H] = 1086.6 |
| 325 | C₅₅H₇₂N₈O₉ | [M + H] = 1057.63 | [M + H] = 1057.4 |
| 326 | C₅₅H₇₂N₈O₉ | [M + H] = 967.52 | [M + H] = 967.5 |
| 327 | C₅₉H₇₉N₁₁O₇S | [M + H] = 973.56 | [M + H] = 973.4 |
| 328 | C₅₉H₇₉N₁₁O₇S | [M + H] = 973.56 | [M + H] = 973.5 |
| 329 | C₅₄H₆₉N₇O₁₁ | [M + H] = 992.52 | [M + H] = 992.4 |
| 330 | C₅₁H₆₅N₇O₉ | [M + H] = 920.49 | [M + H] = 920.6 |
| 331 | C₅₇H₆₉N₇O₉ | [M + H] = 996.53 | [M + H] = 996.6 |
| 332 | C₅₂H₆₇N₇O₉ | [M + H] = 934.51 | [M + H] = 934.6 |

### Biological Assays

Compounds 1-2,4-18A, 19A-19B, 21A-24A, 27-32A, 33-43A, 44-45, 471B-54, 56-59, 68A, 69A, 71B3, 72A, 73-78, 791B-82A, 83-97, 100-110, 112-117, 119-234, 236-294, and 297-332 exhibited: a) a % cross-linking to KRAS^{G12D} of greater than zero within a 24-hour incubation timeframe in the assay described below; and/or b) an IC50 of 2 μM or less in the KRAS^{G12D}-B-Raf (AsPC-1) disruption assay described below.

Potency Assay: pERK

The purpose of this assay is to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H-358 cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCP-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO2 incubator. Test compounds were prepared in 10, 3-old dilutions in DMSO, with a high concentration of 10 mM. On the day of assay, 40 nL of test compound was added to each well of cell culture plate using an Echo550 liquid handler (LabCyte®). Concentrations of test compound were tested in duplicate. After compound addition, cells were incubated 4 hours at 37° C., 5% CO2. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, plate was sealed, and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% paraformaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (pERK, CST-4370, Cell Signaling Technology) was diluted 1:200 in blocking buffer, and 50 μL was added to each well and incubated overnight at 4° C. Cells were washed 4 times for 5 minutes with TBST. Secondary antibody (IR-8000W rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a LI-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

The following compounds exhibited a pERK EC50 of under 5 uM (AsPC-1 KRAS G12D): 179, 157, 178, 327, 205, 106, 242, 121, 183, 36, 158, 196, 84, 17 A and B, 87, 187, 114, 182, 255, 254, 185, 236, 124, 19 7, 1, 107, 192, 34, 118, 296, 78, 89, 104, 74, 306, 310, 105, 152, 269, 229, 221, 294, 117, 119, 240, 151, 193, 86, 245, 12 8, 163, 272, 270, 79 A and B, 232, 140, 138, 293, 38, 94, 110, 172, 271, 246, 72 A and B, 108, 35, 14, 127, 7, 153, 39, 190, 96, 227, 13, 77, 286, 215, 244, 184, 284, 275, 147, 295, 204, 50, 161, 129, 176, 51, 290, 226, 218, 164, 282, 167, 162, 1 31, 228, 292, 233, 308, 304, 48, 9, 113, 298, 277, 54, 57, 219, 173, 220, 268, 49, 149, 247, 120, 154, 307, 56, 166, 11, 53, 101, 10, 8, 238, 97, 303, 132, 186, 52, 297, 93, 85, 83, 280, 103, 200, 276, 278, 144, 165, 199, 33, 139, 112, 224, 177, 2 41, 273, 237, 274, 191, 243, 319, 320, 225, 59, 311, 207, 239, 279, 160, 289, 171, 156, 92, 202, 43A, 266, 208, 281, 15 9, 300, 210, 223, 217, 283, 216, 231, 299, 90, 91, 267, 155, 259, 291, 258, 257, 262, 222, 137, 100, 256, 88, 316, 142, 3 18, 146, 198, 288, 302, 174, 265, 322, 12, 168, 42A, 201, 301, 263, 248, 287, 58, 305, 260, 134, 169, 313, 314, 323, 23 4, 136, 148, 102, 315, 141, 150, 309, 326, 261, 321, 175, 230, 249, 264, 95, 285, 135, 133, 170, 317, 328, 214, 209, 324, 325.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo®2.0 Reagent (Promega).

Cells were seeded at 250 cells/well in 40 μL of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μL) were transferred to the next wells containing 30 μL of 100% DMSO, and repeated until a 9-concentration 3-fold serial dilution was made (starting assay concentration of 10 μM). Test compounds (132.5 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

### *Key:

+++++: IC50≥10 uM+

++++: 10 uM>IC50≥1 uM+

+++: 1 uM>IC50≥0.1 uM+

++: 0.1 uM>IC50≥0.01 uM+

+: IC50<0.01 uM

TABLE 5
H358 Cell Viability assay data (K-Ras G12C, IC50, uM):

| IC50* | Examples of A compounds |
|---|---|
| + | 1, 44, 58, 59, 90, 95, 106, 136, 137, 142, 146, 148, 150, |
| | 155, 156, 159, 160, 165, 171, 174, 175, 186, 201, 207, |
| | 209, 222, 231, 236, 241, 261, 266, 267, 273, 274, 279, |
| | 280, 299, 301, 307, 319, 324, 325, 328, 42A, 43A |
| ++ | 2, 5, 7, 8, 9, 10, 13, 33, 41, 83, 85, 100, 102, 132, 133, |
| | 135, 144, 168, 169, 170, 191, 208, 214, 216, 217, 223, 224, |
| | 225, 226, 227, 228, 230, 234, 239, 248, 249, 256, 257, 258, |
| | 259, 260, 262, 263, 264, 265, 270, 276, 277, 283, 287, 288, |
| | 289, 290, 291, 292, 293, 296, 297, 298, 300, 302, 303, 316, |
| | 317, 322, 323, 326, 42B, 43B |
| +++ | 3, 4, 6, 11, 12, 14, 40, 45, 46, 48, 50, 52, 56, 63, 77, 103, |
| | 107, 141, 202, 210, 243, 285, 294, 304, 313, 318, 320, 321 |
| ++++ | 26, 29, 30, 31, 49, 60, 66, 96, 15 A and B, 16 A and B, 16 |
| | A and B, 17 A and B, 17 A and B, 18 A and B, 18 A and B, |
| | 19 A and B, 19 A and B, 20 A and B, 20 A and B, 22 A and B, |
| | 23 A and B, 24 A and B, 24 A and B, 32 A and B, 32 A and B, |
| | 47 A and B, 62 A and B, 62 A and B, 68 A and B, 68 A and B, |
| | 69 A and B, 69 A and B |
| +++++ | 15 A and B, 25, 27, 28, 47 A and B, 67 |

TABLE 6
AsPC-1 Cell Viability assay data (K-Ras G12D, IC50, uM):

| IC50* | Examples of A compounds |
|---|---|
| + | 209, 325 |
| ++ | 95, 133, 170, 175, 214, 230, 249, 285, 309, 313, 317, 321, 324 |
| +++ | 7, 12, 58, 59, 77, 88, 90, 100, 102, 103, 135, 136, 137, 141, |
| | 142, 144, 146, 148, 150, 155, 156, 159, 160, 168, 169, 171, |
| | 174, 186, 198, 201, 207, 210, 216, 217, 222, 223, 224, 225, |
| | 228, 231, 234, 239, 241, 243, 248, 256, 257, 258, 259, 260, |
| | 261, 262, 263, 264, 265, 266, 267, 270, 273, 274, 277, 279, |
| | 281, 283, 287, 288, 289, 291, 297, 298, 299, 300, 301, 302, |
| | 303, 305, 307, 311, 314, 315, 318, 319, 320, 323, 326, 328, |
| | 19 A and B, 42A, 43A |
| ++++ | 1, 8, 9, 10, 11, 13, 14, 29, 34, 35, 38, 39, 44, 48, 49, 52, |
| | 53, 54, 56, 57, 60, 63, 73, 74, 75, 76, 78, 80, 81, 83, 84, |
| | 85, 86, 87, 91, 92, 96, 97, 101, 104, 105, 106, 107, 108, |
| | 110, 112, 113, 114, 117, 119, 120, 121, 122, 128, 129, 132, |
| | 134, 139, 140, 147, 149, 151, 152, 153, 154, 157, 158, 161, |
| | 162, 163, 164, 165, 166, 167, 172, 173, 176, 177, 184, 185, |
| | 187, 190, 191, 192, 193, 196, 197, 199, 200, 202, 204, 208, |
| | 215, 218, 219, 220, 221, 226, 227, 229, 232, 233, 236, 237, |
| | 238, 240, 242, 244, 245, 246, 247, 255, 268, 269, 272, 275, |
| | 276, 278, 280, 282, 284, 286, 290, 292, 293, 294, 295, 296, |
| | 304, 306, 308, 310, 316, 322, 327, 16 A and B, 16 A and B, |
| | 17 A and B, 17 A and B, 18 A and B, 18 A and B, 22 A and B, |
| | 23 A and B, 32 A and B, 42B, 61 A and B, 61 A and B, |
| | 62 A and B, 62 A and B, 64 A and B, 65 A and B, 65 A and B, |
| | 68 A and B, 69 A and B, 69 A and B, 70 A and B, 71 A and B, |
| | 72 A and B, 72 A and B, 79 A and B, 79 A and B, 82 A and B, |
| | 82 A and B |
| +++++ | 2, 3, 4, 5, 6, 28, 31, 36, 37, 40, 41, 45, 46, 50, 51, 55, |
| | 66, 67, 89, 93, 94, 98, 99, 109, 111, 115, 116, 118, 126, |
| | 127, 130, 131, 138, 143, 145, 178, 179, 180, 181, 182, |
| | 183, 188, 189, 194, 195, 203, 205, 206, 211, 212, 213, |
| | 235, 250, 251, 252, 253, 254, 271, 312, 15 A and B, |
| | 15 A and B, 20 A and B, 20 A and B, 24 A and B, 24 A and B, |
| | 32 A and B, 43B, 47 A and B, 47 A and B, 64 A and B, |
| | 70 A and B, 71 A and B |

Disruption of B-Rat Ras-binding Domain (BRAF^{RBD}) Interaction with K-Ras by Compounds of the Invention (also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides, such as K-Ras G12D and K-Ras G13D.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data is reported as IC50 values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} were combined in a 384-well assay plate at final concentrations of 25 μM, 12.5 nM, and 50 nM, respectively. Compound was present in plate wells as a 10-point 3-fold dilution series starting at a final concentration of 30 μM. After incubation at 25° C. for 3 hours, a mixture of anti-His Eu-W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras:RAF complex were identified as those eliciting a decrease in the TR-FRET ratio relative to DMSO control wells.

### Ras-Raf Disruption/FRET/MOA Assay Data (IC50, uM):

### *Key:

+++++: IC50≥10 uM+

++++: 10 uM>IC50≥1 uM+

+++: 1 uM>IC50≥0.1 uM+

++: 0.1 uM>IC50≥0.01 uM+

+: IC50<0.01 uM

TABLE 7
KRAS G13D FRET data

| IC50* | Examples of A compounds |
|---|---|
| + | None |
| ++ | 12, 58, 83, 85, 86, 88, 89, 95, 100, 109, 113, 115, 120, 129, |
| | 130, 133, 135, 136, 137, 138, 139, 140, 143, 148, 149, 150, |
| | 157, 158, 162, 167, 170, 171, 172, 173, 174, 175, 176, 177, |
| | 182, 186, 193, 194, 195, 196, 197, 198, 204, 209, 230, 231, |
| | 239, 241, 248, 249, 256, 257, 262, 263, 264, 265, 274, 276, |
| | 285, 286, 288, 306, 307, 309, 325, 329, 330, 332, 258*, 259*, |
| | 260*, 261*, 281*, 282*, 283*, 284*, 314*, 315*, 316*, 42A, |
| | 43A |
| +++ | 1, 2, 3, 4, 5, 7, 8, 9, 11, 13, 14, 33, 34, 35, 36, 37, 38, |
| | 39, 40, 41, 44, 45, 48, 49, 50, 51, 52, 53, 54, 56, 57, 59, |
| | 84, 87, 90, 91, 92, 93, 94, 96, 97, 101, 102, 103, 104, 105, |
| | 108, 110, 111, 112, 116, 117, 118, 119, 127, 128, 131, 134, |
| | 141, 142, 144, 145, 146, 147, 151, 152, 153, 155, 156, 159, |
| | 160, 161, 163, 164, 165, 166, 168, 169, 178, 179, 180, 181, |
| | 183, 184, 185, 187, 188, 189, 190, 191, 199, 200, 201, 202, |
| | 203, 205, 206, 207, 208, 212, 213, 214, 215, 216, 217, 218, |
| | 219, 220, 221, 222, 223, 224, 225, 226, 227, 228, 229, 232, |
| | 236, 237, 238, 242, 243, 244, 245, 246, 247, 252, 253, 254, |
| | 255, 266, 267, 268, 269, 270, 271, 272, 273, 275, 277, 278, |
| | 279, 280, 287, 289, 291, 293, 294, 297, 298, 299, 300, 301, |
| | 302, 303, 305, 308, 311, 317, 322, 323, 326, 328, 331, |
| | 47 A and B, 69 A and B |
| ++++ | 6, 10, 25, 26, 27, 28, 29, 30, 31, 46, 55, 66, 67, 73, 74, |
| | 98, 106, 107, 114, 126, 132, 154, 192, 210, 211, 233, |
| | 235, 240, 250, 251, 290, 292, 295, 296, 304, 310, 312, |
| | 313, 318, 319, 320, 321, 324, 15 A and B, 17 A and B, |
| | 18 A and B, 19 A and B, 24 A and B, 32 A and B, 32 A and B, |
| | 42B, 43B, 47 A and B, 62 A and B, 72 A and B |
| +++++ | 60, 63, 75, 76, 77, 78, 80, 81, 99, 121, 122, 123, 124, 125, |
| | 234, 327, 15 A and B, 16 A and B, 16 A and B, 17 A and B, |
| | 18 A and B, 19 A and B, 20 A and B, 20 A and B, 22 A and B, |
| | 23 A and B, 24 A and B, 61 A and B, 61 A and B, 62 A and B, |
| | 64 A and B, 64 A and B, 65 A and B, 65 A and B, 68 A and B, |
| | 68 A and B, 69 A and B, 70 A and B, 70 A and B, 71 A and B, |
| | 71 A and B, 72 A and B, 79 A and B, 79 A and B, 82 A and B, |
| | 82 A and B |

TABLE 8
KRAS G12S FRET data

| IC50* | Examples of A compounds |
|---|---|
| + | 325, 264, 281, 135 |
| ++ | 263, 258, 230, 249, 287, 282, 209, 139, 315, 170, 283, 267, |
| | 150, 260, 274, 284, 42A, 167, 276, 261, 133, 137, 12, 256, |
| | 136, 257, 285, 288, 265, 88, 85, 231, 316, 330, 269, 262, |
| | 280, 148, 186, 278, 182, 248, 259, 309, 162, 317, 314, 332, |
| | 160, 140, 175, 305, 95, 308, 158, 113, 273, 323, 268, 173, |
| | 322, 306, 43A, 214, 177, 171, 161, 146, 272, 207, 329, 157, |
| | 197, 201, 147, 103, 208, 200, 328, 241, 172, 90, 89, 105, |
| | 149, 279, 100, 159, 238, 93, 271, 91, 48, 174, 7, 35, 155, |
| | 307, 53, 36, 52, 92, 190, 156, 237, 302, 326, 59, 8, 142, |
| | 37, 33, 11, 141, 97, 127, 10, 13, 277, 58, 104, 275, 222, |
| | 129, 266, 204, 39, 191, 110, 176, 301, 96, 331, 54, 198, |
| | 239, 130, 255, 134, 83, 50, 1, 34, 289, 86, 38, 47 A and B, |
| | 168, 143, 169, 270, 298 |
| +++ | 14, 115, 152, 109, 49, 102, 223, 189, 44, 291, 144, 297, |
| | 120, 202, 153, 187, 185, 188, 56, 138, 195, 224, 254, 51, |
| | 178, 244, 286, 194, 45, 225, 181, 246, 9, 205, 166, 101, |
| | 196, 252, 2, 116, 243, 193, 216, 253, 179, 293, 217, 192, |
| | 112, 245, 311, 199, 203, 94, 165, 3, 163, 232, 151, 5, |
| | 294, 108, 183, 46, 215, 299, 119, 117, 4, 300, 164, 303, |
| | 184, 213, 227, 145, 57, 128, 304, 6, 220, 229, 218, 226, |
| | 228, 87, 251, 131, 84, 219, 111, 221, 242, 154, 310, 247, |
| | 180, 40, 47 A and B, 41, 290, 118, 236, 292, 31, 324, 296, |
| | 321, 250, 212, 206, 55, 126 |
| ++++ | 210, 240, 77, 121, 211, 319, 42B, 313, 132, 114, 98, |
| | 320, 295, 318, 43B, 235, 122, 107, 62 A and B, 312, |
| | 24 A and B, 106, 74, 234, 78, 66, 15 A and B, 233, |
| | 73, 69 A and B, 63, 29, 67, 22 A and B, 80, 23 A and B |
| +++++ | 25, 124, 32 A and B, 327, 27, 28, 26, 62 A and B, |
| | 72 A and B, 20 A and B, 79 A and B, 19 A and B, |
| | 16 A and B, 123, 24 A and B, 30, 17 A and B, |
| | 15 A and B, 19 A and B, 32 A and B, 18 A and B, |
| | 18 A and B, 17 A and B, 16 A and B, 20 A and B, |
| | 69 A and B, 60, 68 A and B, 68 A and B, 65 A and B, |
| | 65 A and B, 64 A and B, 64 A and B, 70 A and B, |
| | 71 A and B, 70 A and B, 71 A and B, 72 A and B, |
| | 61 A and B, 61 A and B, 82 A and B, 82 A and B, |
| | 81, 79 A and B, 76, 75, 99, 125 |

TABLE 9
KRAS G13C FRET date

| IC50* | Examples of A compounds |
|---|---|
| + | 84, 85, 89, 129, 135, 136, 139, 140, 182, 195, 205, 209, 230, 231, |
| | 256, 257, 258, 260, 282, 283, 284, 285, 325, 329, 330, 42a |
| ++ | 3, 4, 7, 8, 11, 12, 13, 14, 34, 35, 36, 37, 38, 39, 41, 44, 45, 48, 49, |
| | 50, 51, 53, 54, 56, 57, 58, 59, 77, 78, 83, 86, 87, 88, 90, 91, 92, 93, |
| | 94, 95, 96, 97, 100, 102, 103, 104, 105, 108, 109, 112, 113, 115, |
| | 117, 118, 119, 120, 124, 127, 128, 130, 131, 133, 134, 137, 138, |
| | 141, 142, 143, 144, 145, 146, 147, 148, 149, 150, 152, 155, 156, |
| | 157, 158, 159, 160, 161, 162, 167, 169, 170, 171, 172, 173, 174, |
| | 175, 176, 177, 178, 179, 180, 181, 183, 184, 185, 186, 187, 188, |
| | 189, 190, 191, 193, 194, 196, 197, 198, 200, 201, 203, 204, 207, |
| | 208, 212, 213, 214, 215, 232, 237, 238, 239, 241, 245, 246, 248, |
| | 249, 251, 252, 253, 254, 255, 259, 261, 262, 263, 264, 265, 266, |
| | 267, 268, 269, 270, 271, 272, 273, 274, 275, 276, 277, 278, 279, |
| | 280, 281, 286, 287, 288, 297, 301, 305, 306, 307, 308, 309, 311, |
| | 314, 315, 316, 317, 322, 323, 326, 328, 331, 332, 43A, 47 A and |
| | B, 47 A and B, 69 A and B |
| +++ | 1, 2, 5, 6, 9, 10, 31, 33, 40, 46, 52, 55, 66, 74, 76, 98, 101, 106, |
| | 107, 110, 111, 114, 116, 121, 122, 123, 125, 126, 151, 153, 154, |
| | 163, 164, 165, 166, 168, 192, 199, 202, 206, 211, 216, 217, 218, |
| | 219, 220, 221, 222, 223, 224, 225, 226, 227, 228, 229, 233, 236, |
| | 243, 244, 247, 250, 289, 290, 291, 292, 293, 294, 296, 298, 299, |
| | 300, 302, 303, 310, 312, 313, 318, 319, 320, 321, 324, 22 A and |
| | B, 42B, 70 A and B |
| ++++ | 25, 27, 28, 29, 63, 67, 73, 80, 132, 210, 234, 235, 240, 242, 295, |
| | 304, 327, 15 A and B, 16 A and B, 17 A and B, 17 A and B, 18 A |
| | and B, 19 A and B, 20 A and B, 23 A and B, 24 A and B, 32 A and |
| | B, 43B, 62 A and B, 65 A and B, 68 A and B, 69 A and B, 72 A and |
| | B, 79 A and B |
| +++++ | 26, 30, 60, 75, 81, 99, 15 A and B, 16 A and B, 18 A and B, 19 A |
| | and B, 20 A and B, 24 A and B, 32 A and B, 61 A and B, 61 A and |
| | B, 62 A and B, 64 A and B, 64 A and B, 65 A and B, 68 A and B, |
| | 70 A and B, 71 A and B, 71 A and B, 72 A and B, 79 A and B, 82 |
| | A and B, 82 A and B |

TABLE 10
KRAS G12V FRET data

| IC50* | Examples of A compounds |
|---|---|
| + | 325 |
| ++ | 1, 11, 12, 13, 36, 44, 48, 50, 58, 59, 83, 85, 88, 90, 93, 95, 96, |
| | 97, 100, 103, 113, 133, 135, 136, 137, 139, 140, 141, 146, 147, |
| | 148, 149, 150, 151, 155, 156, 157, 158, 159, 160, 161, 162, |
| | 165, 167, 170, 171, 172, 173, 174, 175, 177, 182, 186, 189, |
| | 190, 192, 197, 200, 201, 204, 207, 208, 209, 214, 230, 231, |
| | 239, 241, 248, 249, 256, 257, 258, 259, 260, 261, 262, 263, |
| | 264, 265, 267, 268, 269, 272, 273, 274, 276, 278, 279, 280, |
| | 281, 282, 283, 284, 285, 287, 288, 302, 305, 306, 307, 309, |
| | 314, 315, 316, 317, 322, 323, 326, 328, 329, 330, 331, |
| | 332, 42A, 43A |
| +++ | 2, 3, 4, 5, 6, 7, 8, 9, 10, 14, 31, 33, 34, 35, 37, 38, 39, 40, 41, 45, |
| | 49, 51, 52, 53, 54, 55, 56, 57, 84, 86, 87, 89, 91, 92, 94, 101, |
| | 102, 104, 105, 108, 109, 110, 111, 112, 115, 116, 117, 118, 119, |
| | 120, 127, 128, 129, 130, 131, 134, 142, 143, 144, 145, 152, 153, |
| | 154, 163, 164, 166, 168, 169, 176, 178, 179, 180, 181, 183, 184, |
| | 185, 187, 188, 191, 194, 195, 196, 198, 199, 202, 203, 205, 206, |
| | 212, 213, 215, 216, 217, 218, 219, 220, 221, 222, 223, 224, 225, |
| | 226, 227, 228, 229, 232, 236, 237, 238, 243, 244, 245, 246, 247, |
| | 250, 251, 252, 253, 254, 255, 266, 270, 271, 275, 277, 286, 289, |
| | 290, 291, 292, 293, 294, 297, 298, 299, 300, 301, 303, 304, 308, |
| | 310, 311, 321, 324, 47 A and B |
| ++++ | 29, 46, 66, 67, 74, 77, 78, 80, 98, 106, 107, 114, 121, 122, 123, |
| | 124, 126, 132, 138, 193, 210, 211, 233, 234, 235, 240, 242, 295, |
| | 296, 312, 313, 318, 319, 320, 15 A and B, 16 A and B, 22 A and |
| | B, 24 A and B, 42B, 43B, 47 A and B, 62 A and B, 69 A and B, |
| | 72 A and B, 79 A and B |
| +++++ | 73, 25, 26, 27, 28, 30, 60, 63, 75, 76, 81, 99, 125, 327, 15 A and |
| | B, 16 A and B, 17 A and B, 17 A and B, 18 A and B, 18 A and |
| | B, 19 A and B, 19 A and B, 20 A and B, 20 A and B, 23 A and B, |
| | 24 A and B, 32 A and B, 32 A and B, 61 A and B, 61 A and B, 62 |
| | A and B, 64 A and B, 64 A and B, 65 A and B, 65 A and B, 68 A |
| | and B, 68 A and B, 69A and B, 70A and B, 70 A and B, 71A and |
| | B, 71 A and B, 72 A and B, 79 A and B, 82 A and B, 82 A and B |

TABLE 11
KRAS G12D FRET data

| IC50* | Examples of A compounds |
|---|---|
| + | 214, 305, 325 |
| ++ | 59, 88, 95, 103, 113, 133, 135, 136, 137, 139, 140, 141, 146, 148, |
| | 150, 167, 170, 171, 173, 175, 186, 198, 209, 230, 231, 241, 248, |
| | 249, 256, 258, 260, 261, 263, 264, 265, 267, 269, 274, 276, 278, |
| | 280, 281, 282, 283, 284, 285, 287, 309, 315, 316, 330, 42A, 43A |
| +++ | 1, 2, 7, 8, 12, 13, 14, 33, 34, 35, 36, 37, 38, 39, 40, 41, 44, 45, 48, |
| | 49, 50, 51, 52, 53, 54, 56, 58, 73, 74, 83, 85, 86, 89, 90, 91, 92, 93, |
| | 96, 97, 100, 101, 102, 104, 105, 110, 112, 115, 120, 127, 128, 129, |
| | 130, 131, 134, 138, 142, 143, 144, 147, 149, 151, 152, 153, 155, |
| | 156, 157, 158, 159, 160, 161, 162, 163, 164, 165, 166, 168, 169, |
| | 172, 174, 176, 177, 178, 179, 1 81, 182, 184, 185, 187, 188, 189, |
| | 190, 191, 192, 193, 194, 196, 197, 199, 200, 201, 202, 203, 204, |
| | 207, 208, 215, 216, 217, 222, 223, 224, 225, 227, 236, 237 238, |
| | 239, 242, 243, 244, 245, 252, 253, 254, 255, 257, 259, 262, 266, |
| | 268, 270, 271, 272, 273, 275, 277, 279, 286, 288, 289, 291, 293, |
| | 294, 297, 298, 299, 300, 301, 302, 303, 304, 306, 307, 308, 311, |
| | 313, 314, 317, 321, 322, 323, 324, 326, 328, 329, 331, 332, 70 |
| | A and B |
| ++++ | 16 A and B, 3, 4, 5, 6, 9, 10, 11, 29, 31, 46, 57, 63, 66, 67, 76, 77, |
| | 78, 80, 84, 87, 94, 98, 106, 107, 108, 109, 111, 114, 116, 117, 118, |
| | 119, 121, 122, 123, 124, 125, 126, 132, 145, 154, 180, 183, 195, |
| | 205, 206, 210, 211, 212, 213, 218, 219, 220, 221, 226, 228, 229, |
| | 232, 233, 234, 235, 240, 246, 247, 250, 251, 290, 292, 295, 296, |
| | 310, 312, 318, 319, 320, 327, 15 A and B, 17 A and B, 17 A and |
| | 8, 18 A and B, 19 A and B, 22 Aand B, 23 A and B, 24 A and B, |
| | 32 A and B, 42B, 43B, 47 A and B, 47 A and B, 62 A and B, 68 A |
| | and B, 69 A and B, 72 A and B, 82 A and B |
| +++++ | 25, 26, 27, 28, 30, 55, 60, 75, 81, 99, 15 A and B, 16 A and B, 18 |
| | A and B, 19 A and B, 20 A and B, 20 A and B, 24 A and B, 32 A |
| | and B, 61 A and B, 61 A and B, 62 A and B, 64 A and B, 64 A and |
| | B, 65 A and B, 65 A and B, 68 A and B, 69 A and B, 70 A and B, |
| | 71 A and B, 71 A and B, 72 A and B, 79 A and B, 79 A and B, |
| | 82 A and B |

TABLE 12
KRAS WT FRET data

| IC50* | Examples of A compounds |
|---|---|
| + | 264, 325 |
| ++ | 12, 13, 35, 36, 37, 44, 45, 52, 53, 54, 58, 59, 83, 85, 86, 88, 89, 90, |
| | 91, 92, 93, 95, 96, 97, 100, 102, 103, 105, 109, 110, 112, 113, 115, |
| | 116, 120, 129, 130, 133, 134, 135, 136, 137, 138, 139, 140, 141, |
| | 143, 146, 147, 148, 149, 150, 155, 156, 157, 158, 159, 160, 161, |
| | 162, 167, 168, 169, 170, 171, 172, 173, 174, 175, 176, 177, 182, |
| | 183, 186, 189, 190, 191, 193, 195, 196, 197, 198, 201, 204, 205, |
| | 207, 208, 209, 213, 214, 222, 223, 230, 231, 237, 238, 239, 241, |
| | 245, 246, 248, 249, 252, 253, 256, 257, 258, 259, 260, 261, 262, |
| | 263, 265, 267, 268, 269, 271, 272, 273, 274, 276, 278, 279, 280, |
| | 281, 282, 283, 284, 285, 287, 288, 289, 291, 297, 298, 301, 302, |
| | 305, 306, 307, 308, 309, 314, 315, 316, 317, 322, 323, 326, 328, |
| | 329, 330, 331, 332, 42A, 43A |
| +++ | 1, 2, 3, 4, 5, 6, 7, 8, 9, 10, 11, 14, 33, 34, 38, 39, 40, 41, 48, 49, |
| | 50, 51, 56, 57, 84, 87, 94, 98, 101, 104, 108, 111, 117, 118, 119, |
| | 121, 127, 128, 131, 142, 144, 145, 151, 152, 153, 154, 163, 164, |
| | 165, 166, 178, 179, 180, 181, 184, 185, 187, 188, 192, 194, 199, |
| | 200, 202, 203, 206, 211, 212, 215, 216, 217, 218, 219, 220, 221, |
| | 224, 225, 226, 227, 228, 229, 232, 236, 240, 242, 243, 244, 247, |
| | 250, 251, 254, 255, 266, 270, 275, 277, 286, 290, 292, 293, 294, |
| | 295, 296, 299, 300, 303, 304, 310, 311, 312, 318, 321, 324, 47 |
| | A and B |
| ++++ | 25, 26, 27, 29, 30, 31, 46, 55, 63, 66, 67, 73, 74, 76, 77, 78, 80, |
| | 106, 107, 114, 122, 123, 124, 126, 132, 210, 233, 234, 235, 313, |
| | 319, 320, 327, 15 A and B, 16 A and B, 22 A and B, 23 A and |
| | B, 24 A and B, 42B, 43B, 47 A and B, 62 A and B, 69 A and B, |
| | 79 A and B |
| +++++ | 28, 60, 75, 81, 99, 125, 15 A and B, 16 A and B, 17 A and B, 17 |
| | A and B, 18 A and B, 18 A and B, 19 A and B, 19 A and B, 20 A |
| | and B, 20 A and B, 24 A and B, 32 A and B, 32 A and B, 61 A and |
| | B, 61 A and B, 62 A and B, 64 A and B, 64 A and B, 65 A and B, |
| | 65 A and B, 68 A and B, 68 A and B, 69 A and B, 70 A and B, 70 |
| | A and B, 71 A and B, 71 A and B, 72 A and B, 72 A and B, 79 A |
| | and B, 82 A and B, 82 A and B |

TABLE 13
KRAS G12C FRET data

| IC50* | Examples of A compounds |
|---|---|
| + | 1, 7, 41, 58, 88, 90, 95, 109, 113, 115, 135, 137, 139, 140, 142, 146, |
| | 148, 149, 150, 151, 156, 161, 162, 165, 167, 171, 174, 175, 183, |
| | 184, 186, 190, 192, 196, 198, 200, 201, 203, 204, 209, 230, 231, |
| | 241, 249, 252, 263, 264, 266, 267, 268, 269, 270, 271 , 272, 273, |
| | 274, 280, 281, 306, 307, 309, 311, 312, 325, 329, 330, 331, 42A, |
| | 43A |
| ++ | 2, 3, 4, 5, 6, 8, 9, 10, 11, 12, 13, 14, 31, 33, 34, 35, 36, 37, 38, 39, |
| | 44, 45, 48, 49, 50, 51, 52, 53, 54, 56, 59, 73, 74, 77, 78, 83, 84, 85, |
| | 86, 87, 89, 91, 92, 93, 94, 96, 97, 100, 101, 102, 103, 104, 105, 106, |
| | 110, 114, 117, 119, 124, 125, 128, 129, 130, 131, 133, 134, 136, |
| | 138, 141, 143, 144, 147, 152, 153, 154, 155, 157, 158, 159, 160, |
| | 168, 169, 170, 172, 173, 176, 177, 178, 179, 181, 182, 185, 188, |
| | 189, 191, 193, 195, 197, 202, 205, 206, 207, 208, 211, 213, 214, |
| | 215, 216, 217, 220, 222, 223, 224, 225, 232, 236, 237, 238, 239, |
| | 243, 244, 245, 246, 247, 248, 250, 251, 253, 254, 255, 256, 257, |
| | 258, 259, 260, 261, 262, 265, 275, 276, 277, 278, 279, 282, 283, |
| | 284, 285, 287, 288, 289, 291, 293, 294, 297, 298, 299, 301, 302, |
| | 305, 308, 314, 315, 316, 317, 319, 322, 323, 326, 328, 332, 16 A |
| | and B, 17 A and B, 18 A and B, 22 A and B, 23 A and B, 32 A |
| | and B, 43B, 68 A and B, 69 A and B, 82 A and B |
| +++ | 40, 46, 55, 57, 66, 67, 75, 76, 80, 81, 98, 107, 108, 111, 112, 116, |
| | 118, 120, 121, 122, 123, 126, 127, 132, 145, 163, 164, 166, 180, |
| | 187, 194, 199, 210, 212, 21 8, 219, 221, 226, 227, 228, 229, 233, |
| | 234, 235, 240, 242, 286, 290, 292, 295, 296, 300, 303, 304, 310, |
| | 313, 318, 320, 321, 324, 15 A and B, 16 A and B, 17 Aand B, 19 |
| | A and B, 20 A and B, 24 A and B, 42B, 47 A and B, 61 A and B, |
| | 62 A and B, 70 A and B, 72 A and B, 79 A and B |
| ++++ | 25, 26, 27, 28, 29, 63, 327, 18 A and B, 32 A and B, 47 A and B, |
| | 62 A and B, 65 A and B, 68 A and B, 69 A and B, 71 A and B, |
| | 72 A and B, 82 A and B |
| +++++ | 30, 60, 99, 15 A and B, 19 A and B, 20 A and B, 24 A and B, 61 A |
| | and B, 64 A and B, 64 A and B, 65 A and B, 70 A and B, 71 A and |
| | B, 79 A and B |

TABLE 14
KRAS Q61H FRET data

| IC50* | Examples of A compounds |
|---|---|
| + | 209, 230, 231, 246, 249, 258, 264, 281, 325 |
| ++ | 36, 37, 91, 92, 108, 117, 119, 141, 144, 163, 164, 166, 167, 170, |
| | 186, 202, 203, 204, 205, 207, 208, 212, 213, 214, 215, 216, 217, |
| | 218, 220, 222, 223, 224, 225, 227, 228, 229, 232, 237, 238, 239, |
| | 241 244, 245, 248, 252, 253, 254, 255, 256, 257, 259, 260, 261, |
| | 262, 263, 265, 266, 267, 268, 269, 271, 272, 273, 274, 275, 276, |
| | 277, 278, 279, 280, 282, 283, 284, 285, 286, 287, 288, 289, 291, |
| | 293, 294, 297, 298, 299, 300, 301, 302, 303, 304, 305, 306, 307, |
| | 308, 309, 311, 314, 315, 316, 317, 322, 323, 326, 328, 329, 330, |
| | 331, 332 |
| +++ | 165, 206, 210, 211, 219, 221, 226, 233, 235, 236, 240, 242, 243, |
| | 247, 250, 251, 270, 290, 292, 295, 296, 310, 312, 31 3, 318, 319, |
| | 320, 321, 324 |
| ++++ | 234, 327 |
| +++++ | None |

TABLE 15
NRAS G12C FRET data

| IC50* | Examples of A compounds |
|---|---|
| + | 1, 5, 7, 41, 58, 59, 86, 88, 90, 95, 109, 113, 115, 135, 136, 137, |
| | 138, 139, 140, 142, 146, 148, 149, 150, 151, 155, 156, 159, 160, |
| | 161, 162, 165, 167, 171, 174, 175, 184, 186, 192, 196, 198, 200, |
| | 201, 203, 204, 209, 230, 231, 241, 249, 252, 263, 264, 266, 267, |
| | 268, 269, 270, 271, 272, 273, 274, 276, 278, 280, 281, 284, 306, |
| | 307, 309, 311, 312, 325, 329, 330, 42A, 43A |
| ++ | 2, 3, 6, 8, 9, 10, 11, 12, 13, 14, 33, 34, 35, 36, 37, 38, 39, 44, 45, |
| | 48, 49, 50, 52, 53, 54, 73, 74, 77, 78, 83, 84, 85, 87, 89, 91, 92, |
| | 93, 96, 97, 100, 101, 102, 103, 104, 105, 106, 110, 114, 124, 125, |
| | 128, 129, 130, 131, 133, 134, 141, 143, 144, 147, 152, 153, 154, |
| | 157, 158, 168,169, 170, 172, 173, 176, 177, 178, 179, 181, 182, |
| | 183, 185, 188, 189, 190, 191, 193, 195, 197, 202, 205, 206, 207, |
| | 208, 211, 213, 214, 215, 216, 217, 218, 222, 223, 224, 225, 227, |
| | 228, 232, 236, 237, 238, 239, 243, 244, 245, 246, 247, 248, 251, |
| | 253, 254, 255, 256, 257, 258, 259, 260, 261, 262, 265, 275, |
| | 277, 279, 282, 283, 285, 287, 288, 289, 291, 293, 294, 297, 298, |
| | 299, 300, 301, 302, 305, 308, 314, 315, 316, 317, 319, 322, 323, |
| | 326, 328, 331, 332, 16 A and B, 17 A and B, 18 A and B, 22 |
| | A and B, 23 A and B, 43B, 68 A and B, 69 A and B |
| +++ | 219, 4, 31, 40, 46, 51, 56, 57, 66, 67, 75, 76, 80, 94, 107, 108, 111, |
| | 112, 116, 117, 118, 119, 120, 121 ,122, 123, 126, 127, 132, 145, |
| | 163, 164, 166, 180, 187, 194, 199, 210, 212, 220, 221, 226, 229, |
| | 233, 235, 240, 242, 250, 286, 290, 292, 295, 296, 303, 304, |
| | 310, 313, 320, 321, 324, 15 A and B, 16 A and B, 17 A and B, |
| | 19 A and B_{,} 20 A and B, 32 A and B, 42B, 47 A and B, 61 |
| | A and B, 62 A and B, 70 A and B, 71 A and B, 72 A and B, |
| | 72 A and B, 79 A and B, 82 A and B |
| ++++ | 318, 26, 28, 29, 63, 81, 98, 234, 327, 18 A and B, 24 A and B_{,} 47 |
| | A and B, 62 A and B, 68 A and B, 69 A and B, 82 A and B |
| +++++ | 25, 27, 30, 60, 99, 15 A and B, 19 A and B, 20 A and B, 24 A |
| | and B, 32 A and B, 61 A and B, 64 A and B, 64 A and B, 65 A |
| | and B, 65 A and B_{,} 70 A and B, 71 A and B, 79 A and B |

TABLE 16
NRAS WT FRET data

| IC50* | Examples of A compounds |
|---|---|
| + | 258, 264, 315, 325 |
| ++ | 37, 91, 92, 117, 141, 167, 170, 186, 204, 205, 207, 208, 209, 213, |
| | 214, 217, 222, 223, 224, 225, 230, 231, 237, 238, 239, 241, 245, |
| | 246, 248, 249, 252, 253, 255, 256, 257, 259, 260, 261, 262, 263, |
| | 265, 207, 268, 269, 272, 273, 274, 276, 278, 279, 280, 281, 282, |
| | 283, 284, 285, 287, 288, 289, 291, 297, 298, 301, 302, 305, 306, |
| | 307, 308, 309, 311, 314, 316, 317, 322, 323, 326, 328, 329, 330, |
| | 331, 332 |
| +++ | 36, 108, 119, 144, 163, 164, 165, 166, 202, 203, 206, 211, 212, |
| | 215, 216, 218, 219, 220, 221, 226, 227, 228, 229, 232, 236, 242, |
| | 243, 244, 247, 250, 251, 254, 266, 270, 271, 275, 277, 286, 290, |
| | 292, 293, 294, 295, 296, 299, 300, 303, 304, 310, 312, 321 , 324 |
| ++++ | 210, 233, 234, 235, 240, 313, 318, 319, 320 |
| +++++ | 327 |

TABLE 17
NRAS Q61K FRET data

| IC50* | Examples of A compounds |
|---|---|
| + | None |
| ++ | 167, 170, 216, 217, 218, 219, 220, 222, 223, 224, 225, 227, |
| | 228, 229, 231, 246, 249, 258, 260, 264, 281, 289, 291, 293, |
| | 294, 297, 298, 299, 300, 301, 302, 315, 325, 326, 329 |
| +++ | 36, 37, 91, 92, 108, 117, 119, 141, 163, 186, 202, 203, 204, |
| | 205, 207, 208, 209, 212, 213, 214, 221, 226, 230, 232, 235, |
| | 237, 238, 239, 241, 245, 248, 252, 253, 254, 255, 256, 257, |
| | 259, 261, 262, 263, 265, 266, 267, 268, 269, 271, 272, 273, |
| | 274, 275, 276, 277, 278, 279, 280, 282, 283, 284, 285, 286, |
| | 287, 288, 290, 292, 295, 296, 303, 304, 305, 306, 307, 308, |
| | 309, 310, 311, 314, 316, 317, 319, 322, 323, 324, 328, 330, |
| | 331, 332 |
| ++++ | 144, 164, 165, 166, 206, 210, 211, 215, 233, 234, 236, 242, |
| | 243, 244, 247, 250, 251, 270, 312, 313, 318, 320, 321 |
| +++++ | 240, 327 |

TABLE 18
NRAS Q61R FRET data

| IC50* | Examples of A compounds |
|---|---|
| + | None |
| ++ | 170, 209, 222, 223, 224, 230, 249, 258, 264, 289, 297, 298, 301, |
| | 302, 325, 326, 329 |
| +++ | 36, 37, 91, 92, 108, 117, 141, 163, 167, 186, 202, 203, 204, 205, |
| | 207, 208, 212, 213, 214, 216, 217, 218, 219, 220, 221, 225, 226, |
| | 227, 228, 229, 231, 232, 237, 238, 239, 241, 244, 245, 246, 248, |
| | 252, 253, 254, 255, 256, 257, 259, 260, 261, 262, 263, 265, 266, |
| | 267, 268, 269, 271, 272, 273, 274, 275, 276, 278, 279, 280, 281, |
| | 282, 283, 284, 285, 286, 287, 288, 290, 291, 292, 293, 294, 296, |
| | 299, 300, 303, 304, 305, 306, 307, 308, 309, 310, 311, 314, 315, |
| | 316, 317, 322, 323, 324, 328, 330, 331, 332 |
| ++++ | 119, 144, 164, 165, 166, 206, 210, 211 , 215, 234, 235, 236, 240, |
| | 242, 243, 247, 250, 251, 270, 277, 295 ,312, 313, 318, 319, 320, |
| | 321 |
| +++++ | 233, 327 |

Cross-Linking of Ras Proteins with Compounds of the Invention to Form Conjugates

Note—The following protocol describes a procedure for monitoring cross-linking of K-Ras G12C (GMP-PNP) to a compound of the invention. This protocol may also be executed substituting other Ras proteins or nucleotides, such as such as K-Ras G12D and K-Ras G13D.

The purpose of this biochemical assay was to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A, and 2 μM test compound, a 5 μM stock of GMP-PNP-loaded K-Ras (1-169) G12C was diluted 10-fold to yield a final concentration of 0.5 μM; with final sample volume being 100 μL.

The sample was incubated at 25° C. for a time period of up to 24 hours prior to quenching by the addition of 10 μL of 5% Formic Acid. Quenched samples were centrifuged at 15000 rpm for 15 minutes in a benchtop centrifuge before injecting a 10 μL aliquot onto a reverse phase C4 column and eluting into the mass spectrometer with an increasing acetonitrile gradient in the mobile phase. Analysis of raw data was carried out using Waters MassLynx MS software, with % bound calculated from the deconvoluted protein peaks for labeled and unlabeled K-Ras.

### In Vitro Cell Proliferation Panels

Potency for inhibition of cell growth was assessed at CrownBio using standard methods. Briefly, cell lines were cultured in appropriate medium, and then plated in 3D methylcellulose. Inhibition of cell growth was determined by CellTiter-Glo® after 5 days of culture with increasing concentrations of compounds. Compound potency was reported as the 50% inhibition concentration (absolute IC50). The assay took place over 7 days. On day 1, cells in 2D culture were harvested during logarithmic growth and suspended in culture medium at 1×105 cells/mi. Higher or lower cell densities were used for some cell lines based on prior optimization. 3.5 ml of cell suspension was mixed with 6.5% growth medium with 1% methylcellulose, resulting in a cell suspension in 0.65% methylcellulose. 90 μl of this suspension was distributed in the wells of 2 96-well plates. One plate was used for day 0 reading and 1 plate was used for the end-point experiment. Plates were incubated overnight at 37 C with 5% 002. On day 2, one plate (for t0 reading) was removed and 10 μl growth medium plus 100 μl CellTiter-Glo® Reagent was added to each well. After mixing and a 10 minute incubation, luminescence was recorded on an EnVision Multi-Label Reader (Perkin Elmer). Compounds in DMSO were diluted in growth medium such that the final, maximum concentration of compound was 10 μM, and serial 4-fold dilutions were performed to generate a 9-point concentration series. 10 μl of compound solution at 10 times final concentration was added to wells of the second plate. Plate was then incubated for 120 hours at 370 and 5% 002. On day 7 the plates were removed, 100 μl CellTiter-Glo® Reagent was added to each well, and after mixing and a 10 minute incubation, luminescence was recorded on an EnVision Multi-Label Reader (Perkin Elmer). Data was exported to GeneData Screener and modeled with a sigmoidal concentration response model in order to determine the IC50 for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite having a KRAS G20 mutation, is insensitive to the KRAS G120 (OFF) inhibitor MRTX-849 (Hallin et al., Cancer Discovery 10:54-71 (2020)), and the cell line SW1573, which is insensitive to the KRAS G12C (OFF) inhibitor AMG510 (Canon et al., Nature 575:217-223 (2019)).

TABLE 19
IC50 values for various cancer cell lines with Compound B

| Cell Line | Histotype | Cancer Driver/Mutant | IC50* |
|---|---|---|---|
| A-375 | Skin | BRAF V600E | low sensitivity |
| KYSE-410 | HN/Esophagus | KRAS G12C | moderately |
| | | | sensitive |
| MIA PaCa-2 | Pancreas | KRAS G12C | very sensitive |
| NCI-H358 | Lung | KRAS G12C | very sensitive |
| SW1573 | Lung | KRAS G12C | low sensitivity |
| SW837 | Intestine/Large/ | KRAS G12C | very sensitive |
| | Colorectum | | |
| LS513 | Intestine/Large/ | KRAS G12D | moderately |
| | Colorectum | | sensitive |
| HuCCT1 | Liver/Bile duct | KRAS G12D | very sensitive |
| HCC1588 | Lung | KRAS G12D | low sensitivity |
| HPAC | Pancreas | KRAS G12D | very sensitive |
| AsPC-1 | Pancreas | KRAS G12D | moderately |
| | | | sensitive |
| AGS | Stomach | KRAS G12D | very sensitive |
| HEC-1-A | Uterus | KRAS G12D | very sensitive |
| SW403 | Intestine/Large/ | KRAS G12V | moderately |
| | Colorectum | | sensitive |
| NOZ | Liver/Bile duct | KRAS G12V | low sensitivity |
| NCI-H441 | Lung | KRAS G12V | moderately |
| | | | sensitive |
| NCI-H727 | Lung | KRAS G12V | moderately |
| | | | sensitive |
| OVCAR-5 | Ovary | KRAS G12V | moderately |
| | | | sensitive |
| Capan-2 | Pancreas | KRAS G12V | moderately |
| SW48 | Intestine/Large/ | not MAPK | sensitive |
| | Colorectum | (PIK3CAG914R, | |
| | | EGFR G719S) | |
| NCI-H2009 | Lung | other KRAS (G12A) | moderately |
| | | | sensitive |
| CAL-62 | HN/Thyroid | other KRAS (G12R) | low sensitivity |
| A549 | Lung | other KRAS (G12S) | low sensitivity |
| TOV-21G | Ovary | other KRAS (G13C) | low sensitivity |
| DV-90 | Lung | other KRAS (G13D) | low sensitivity |
| HCT116 | Intestine/Large/ | other KRAS (G13D) | moderately |
| | Colorectum | | sensitive |
| NCI-H747 | Intestine/Large/ | other KRAS (G13D) | moderately |
| | Colorectum | | sensitive |
| NCI-H460 | Lung | other KRAS (Q61H) | moderately |
| | | | sensitive |
| Calu-6 | Lung | other KRAS (Q61K) | moderately |
| | | | sensitive |
| SNU-668 | Stomach | other KRAS (Q61K) | moderately |
| | | | sensitive |
| OZ | Liver/Bile duct | other KRAS (Q61L) | moderately |
| | | | sensitive |
| SW948 | Intestine/Large/ | other KRAS (Q61L) | low sensitivity |
| | Colorectum | | |
| BxPC-3 | Pancreas | other MARK (BRAF | low sensitivity |
| | | V487 P492delinsA) | |
| NCI-H1975 | Lung | other MARK (EGFR | moderately |
| | | T790M, L858R) | sensitive |
| NCI-H3122 | Lung | other MARK (EML4- | moderately |
| | | ALK (E13, A20)) | sensitive |
| YCC-1 | Stomach | other MARK (KRAS | |
| | | Amp) | |
| MeWo | Skin | other MARK | low sensitivity |
| | | (NF1 mut) | |
| NCI-H1838 | Lung | other MARK | moderately |
| | | (NF1 mut) | sensitive |

*Key:
low sensitivity: IC50 ≥ 1 uM
moderately sensitive: 1 uM > IC50 ≥ 0.1 uM
very sensitive: IC50 < 0.1 uM
blank = not measured

In Vivo PD and Efficacy Data with Compound A, a Compound of the Present Invention

### FIG. 1A:

Methods: The human pancreatic adenocarcinoma HPAC KRAS G12D/wt xenograft model was used for a single-dose PD study. Compound A (AsPC-1 pERK K-Ras G12D EC5: 0.036 uM) was administered at 30 and 60 mg/kg by intraperitoneal injection (ip injection). The treatment groups with sample collections at various time points were summarized in Table 20 below. Tumor samples were collected to assess RAS/ERK signaling pathway modulation by measuring the mRNA level of human DUSP6 in qPCR assay.

TABLE 20
Summary of treatment groups, doses, and time points for single-dose
PD study using HPAC tumors.

| Compound/group | Dose/Regimen | PD, n = 3/time point |
|---|---|---|
| Vehicle control | 10 ml/kg ip | 1 h, 24 h |
| Compound A | 30 mg/kg ip | 1 h, 4 h, 8 h, 24 h |
| Compound A | 60 mg/kg ip | 1 h, 4 h, 6 h, 24 h |

Results: In FIG. 1A, Compound A at either 30 mg/kg or 60 mg/kg led to inhibition of DUSP6 mRNA levels in tumors at all time points tested, indicating strong MAPK pathway modulation. The inhibitory effects of Compound A on DUSP6 mRNA levels are durable even 24 hours after drug administration.

### FIG. 11B:

Methods: Effects of Compound A on tumor cell growth in vivo were evaluated in the human pancreatic adenocarcinoma HPAC KRAS G12D/wt xenograft model using female BALB/c nude mice (6-8 weeks old). Mice were implanted with HPAC tumor cells in PBS (3×106 cells/mouse) subcutaneously in the flank. Once tumors reached an average size of ˜150 mm3, mice were randomized to treatment groups to start the administration of test articles or vehicle. Compound A was administered by intraperitoneal injection once daily. Body weight and tumor volume (using calipers) was measured twice weekly until study endpoints.

Results: Single-agent Compound A administered at 10 mg/kg ip daily led to a TGI of 89.9% at Day 28, while both 30 mg/kg and 60 mg/kg Compound A dosed ip daily resulted in complete regression of all tumors in the group (complete regression defined as >85% tumor regression from baseline) at the end of treatment (Day 35 after treatment started) in HPAC CDX model with heterozygous KRAS G12D. The anti-tumor activity of all 3 tested doses of Compound A was statistically significant compared with control group (***p<0.001, ordinary One-way ANOVA with multiple comparisons via a post-hoc Tukey's test).

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### APPENDIX B-1

### Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### SUMMARY

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

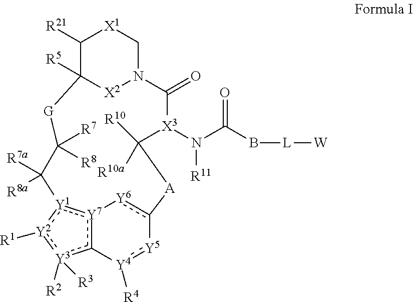

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R²¹ is hydrogen or C₁-C₃ alkyl (e.g., methyl).

Also provided are pharmaceutical compositions comprising a compound of Formula I, or a pharmaceutically acceptable salt thereof, and a pharmaceutically acceptable excipient.

Further provided is a conjugate, or salt thereof, comprising the structure of Formula IV:

M-L-P Formula IV

wherein L is a linker;

P is a monovalent organic moiety; and

M has the structure of Formula V:

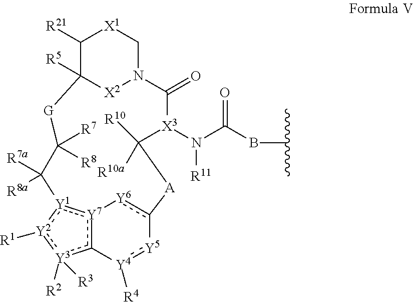

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R²¹ is H or C₁-C₃ alkyl.

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE DRAWINGS

FIG. 1A and FIG. 1B: These figures illustrate a matched pair analysis of potencies of certain compounds of the present invention (Formula BB) (points on the right) and corresponding compounds of Formula AA (points on the left) wherein a H is replaced with (S)Me in the context of two different cell-based assays. The y axes represent pERK EC50 (FIG. 1A) or CTG IC50 (FIG. 1B) as measured in an H358 cell line.

FIG. 2A and FIG. 2B: A compound of the present invention, Compound A, drove deep regressions in vivo in a NSCLC (KRAS G12C) xenograft model. Some animals exhibited complete responses (CR)=3 consecutive tumor measurements ≤30 mm3. FIG. 2A shows Compound A dosed at 100 mg/kg by daily oral gavage led to tumor regression in NCI-H358 KRASG12C xenograft model, which is a sensitive model to KRASG12C inhibition alone. The spaghetti titer plot (FIG. 2B) displaying individual tumor growth is shown next to the tumor volume plot (FIG. 2A).

FIG. 3A and FIG. 3B: A compound of the present invention, Compound B, drove tumor xenograft regressions in combination with a MEK inhibitor, cobimetinib, in a NSCLC (KRAS G12C) model. FIG. 3A shows the combination of intermittent intravenous administration of Compound B at 50 mg/kg plus daily oral administration of cobimetinib at 2.5 mg/kg drove tumor regression, whereas each single agent led to tumor growth inhibition. End of study responses were shown as waterfall plots (FIG. 3B), which indicate 6 out 10 mice had tumor regression in the combination group, whereas no tumor regressions recorded in each single agent group.

FIG. 4A and FIG. 4B: A compound of the present invention, Compound C, dosed weekly with daily SHP2 inhibitor, RMC-4550, drove xenograft regressions in a NSCLC (KRAS G12C) model. In FIG. 4A, the combinatorial activity of once weekly intravenous administration of Compound C at 60 mg/kg plus daily oral administration of SHP2 inhibitor at 30 mg/kg is shown. End of study responses in individual tumors were plotted as a waterfall plot (FIG. 4B).

FIG. 5: A compound of the present invention, Compound D, combined with a MEK inhibitor, trametinib, suppressed in vitro growth durably in a long-term cell growth NSCLC (KRAS G12C) model.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and Ce alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O) NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)_{0- 4}S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —N O₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH^{•}₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

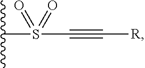

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

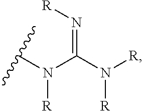

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting moiety B to moiety W in a compound of Formula I, such that the resulting compound is capable of achieving an IC50 of 2 uM or less in the Ras-RAF disruption assay protocol provided in the Examples below, and provided here:

The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

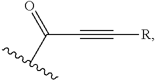

wherein R is any any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that both covalent and non-covalent interactions of a compound of the present invention with Ras and the chaperone protein (e.g., cyclophilin A) may contribute to the inhibition of Ras activity. In some embodiments, a compound of the present invention forms a covalent adduct with a side chain of a Ras protein (e.g., a sulfhydryl side chain of the cysteine at position 12 or 13 of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art. One method of determining covalent adduct formation is to perform a “cross-linking” assay, such as under these conditions (Note—the following protocol describes a procedure for monitoring cross-linking of K-Ras G12C (GMP-PNP) to a compound of the invention. This protocol may also be executed substituting other Ras proteins or nucleotides).

The purpose of this biochemical assay is to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A and 2 μM test compound, a 5 μM stock of GMP-PNP-loaded K-Ras (1-169) G12C is diluted 10-fold to yield a final concentration of 0.5 μM; with final sample volume being 100 μL.

The sample is incubated at 25° C. for a time period of up to 24 hours prior to quenching by the addition of 10 μL of 5% Formic Acid. Quenched samples are centrifuged at 15000 rpm for 15 minutes in a benchtop centrifuge before injecting a 10 μL aliquot onto a reverse phase C4 column and eluting into the mass spectrometer with an increasing acetonitrile gradient in the mobile phase. Analysis of raw data may be carried out using Waters MassLynx MS software, with % bound calculated from the deconvoluted protein peaks for labeled and unlabeled K-Ras.

Accordingly, provided herein is a compound, or pharmaceutically acceptable salt thereof, having the structure of Formula I:

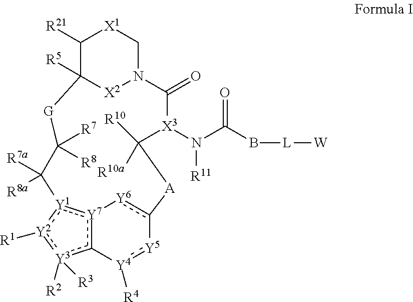

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R²¹ is hydrogen or C₁-C₃ alkyl (e.g., methyl).

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, R²¹ is hydrogen.

In some embodiments, provided herein is a compound, or pharmaceutically acceptable salt thereof, having the structure of Formula Ia:

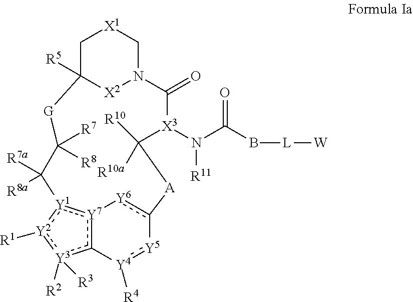

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo; and

R¹¹ is hydrogen or C₁-C₃ alkyl.

In some embodiments, the disclosure features a compound, or pharmaceutically acceptable salt thereof, of structural Formula Ib:

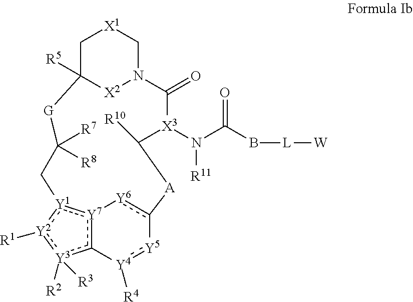

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments of compounds of the present invention, G is optionally substituted C₁-C₄ heteroalkylene.

In some embodiments, a compound having the structure of Formula Ic is provided, or a pharmaceutically acceptable salt thereof:

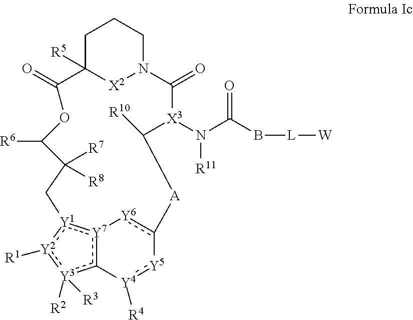

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments of compounds of the present invention, X² is NH. In some embodiments, X³ is CH. In some embodiments, R¹¹ is hydrogen. In some embodiments, R¹¹ is C₁-C₃ alkyl. In some embodiments, R¹¹ is methyl.

In some embodiments, a compound of the present invention has the structure of Formula id, or a pharmaceutically acceptable salt thereof:

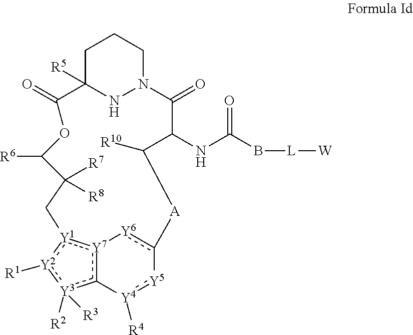

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of a compound of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—. In some embodiments, R⁵ is hydrogen. In some embodiments, R⁵ is C₁-C₄ alkyl optionally substituted with halogen. In some embodiments, R⁵ is methyl. In some embodiments, Y⁴ is C. In some embodiments, R⁴ is hydrogen. In some embodiments, Y⁵ is CH.

In some embodiments, Y⁶ is CH. In some embodiments, Y¹ is C. In some embodiments, Y² is C. In some embodiments, Y³ is N. In some embodiments, R³ is absent. In some embodiments, Y⁷ is C.

In some embodiments, a compound of the present invention has the structure of Formula Ie, or a pharmaceutically acceptable salt thereof:

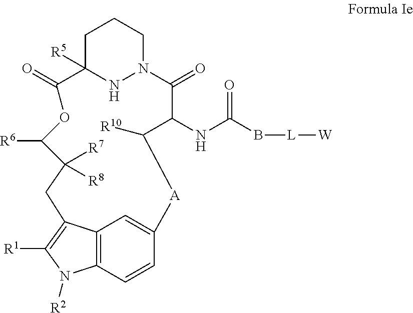

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of a compound of the present invention, R⁶ is hydrogen. In some embodiments, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl. In some embodiments, R² is fluoroalkyl. In some embodiments, R² is ethyl. In some embodiments, R₂ is —CH₂CF₃. In some embodiments, R₂ is C₂-C₆ alkynyl. In some embodiments, R₂ is —CHC≡CH. In some embodiments, R₂ is —CH₂C≡CCH₃. In some embodiments, R⁷ is optionally substituted C₁-C₃ alkyl. In some embodiments, R⁷ is C₁-C₃ alkyl. In some embodiments, R⁸ is optionally substituted C₁-C₃ alkyl. In some embodiments, R⁸ is C₁-C₃ alkyl.

In some embodiments, a compound of the present invention has the structure of Formula If, or a pharmaceutically acceptable salt thereof:

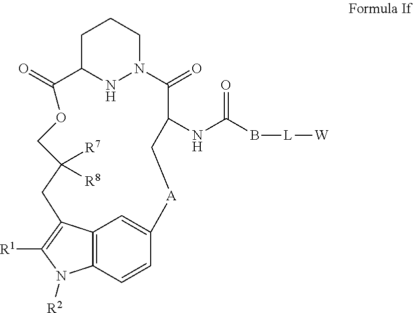

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a compound of the present invention, R¹ is optionally substituted 6 to 10-membered aryl, optionally substituted 3 to 6-membered cycloalkenyl, or optionally substituted 5 to 10-membered heteroaryl. In some embodiments, R¹ is optionally substituted 6-membered aryl, optionally substituted 6-membered cycloalkenyl, or optionally substituted 6-membered heteroaryl.

In some embodiments of a compound of the present invention, R¹ is

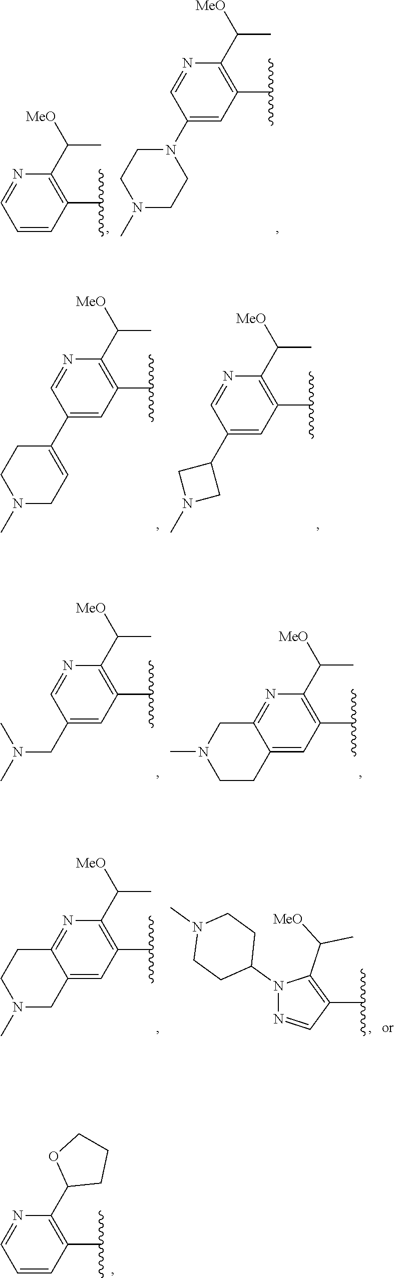

or a stereoisomer (e.g., atropisomer) thereof.

In some embodiments of a compound of the present invention, R₁ is

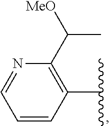 [SMILES: *c1cccnc1C(C)OC]

or a stereoisomer (e.g., atropisomer) thereof. In some embodiments of a compound of the present invention, R₁ is

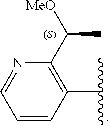

In some embodiments, a compound of the present invention has the structure of Formula Ig, or a pharmaceutically acceptable salt thereof:

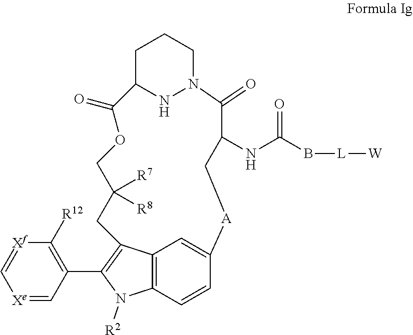

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

R² is C₁-C₆ alkyl, C₁-C₆ fluoroalkyl, or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl

X^{e} and X^{f} are, independently, N or CH; and

R¹² is optionally substituted C₁-C₆ alkyl, optionally substituted C₁-C₆ heteroalkyl, or optionally substituted 3 to 6-membered heterocycloalkylene.

In some embodiments of a compound of the present invention, X^{e} is N and X^{f} is CH. In some embodiments, X^{e} is CH and X^{f} is N.

In some embodiments of a compound of the present invention, R¹² is optionally substituted C₁-C₆ heteroalkyl. In some embodiments, R¹² is

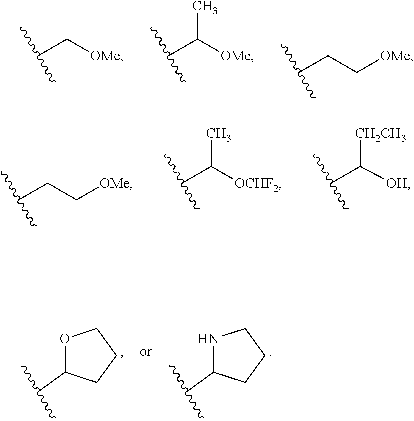

In some embodiments, R¹² is

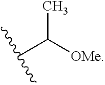 [SMILES: *C(C)OC]

In some embodiments, a compound of the present invention has the structure of Formula VI, or a pharmaceutically acceptable salt thereof:

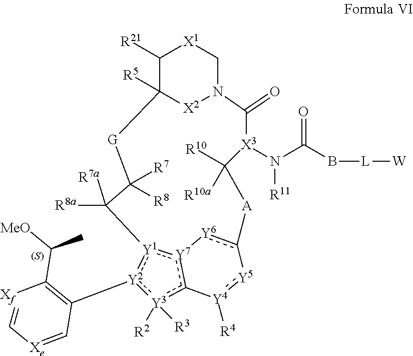

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₃ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl;

R²¹ is hydrogen or C₁-C₃ alkyl (e.g., methyl); and

X^{e} and X^{f} are, independently, N or CH.

In some embodiments, a compound of the present invention has the structure of Formula VIa, or a pharmaceutically acceptable salt thereof:

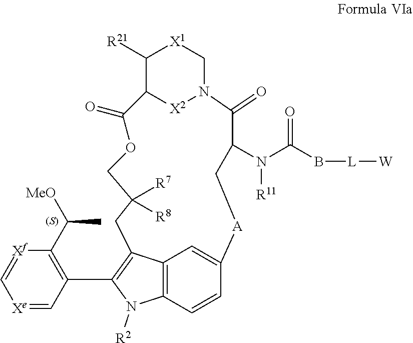

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

R² is C₁-C₆ alkyl, C₁-C₆ fluoroalkyl, or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} and X^{f} are, independently, N or CH;

R¹¹ is hydrogen or C₁-C₃ alkyl; and

R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of Formula VIb, or a pharmaceutically acceptable salt thereof:

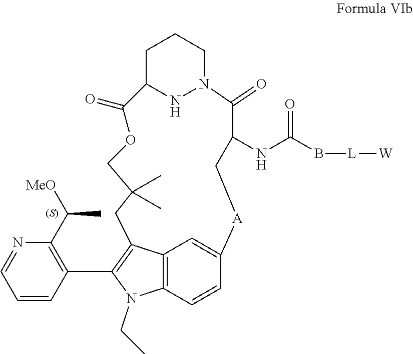

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

L is absent or a linker; and

W is a cross-linking group comprising a vinyl ketone, a vinyl sulfone, an ynone, or an alkynyl sulfone. In some embodiments of a compound of the present invention, A is optionally substituted 6-membered arylene.

In some embodiments, a compound of the present invention has the structure of Formula VIc (corresponding for Formula BB of FIG. 1A and FIG. 1B), or a pharmaceutically acceptable salt thereof:

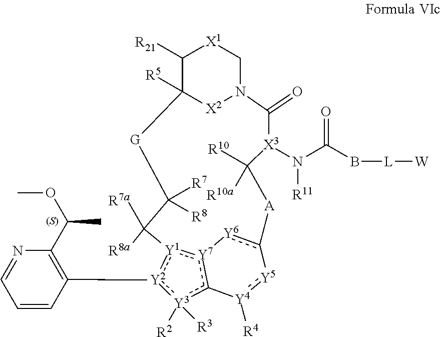

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R²¹ is hydrogen or C₁-C₃ alkyl (e.g., methyl).

In some embodiments, A has the structure:

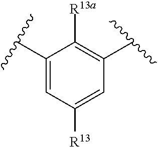

wherein R¹³ is hydrogen, halo, hydroxy, amino, optionally substituted C₁-C₆ alkyl, or optionally substituted C₁-C₆ heteroalkyl; and R^{13a} is hydrogen or halo. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments, A is optionally 3 substituted 5 to 6-membered heteroarylene. In some embodiments A is:

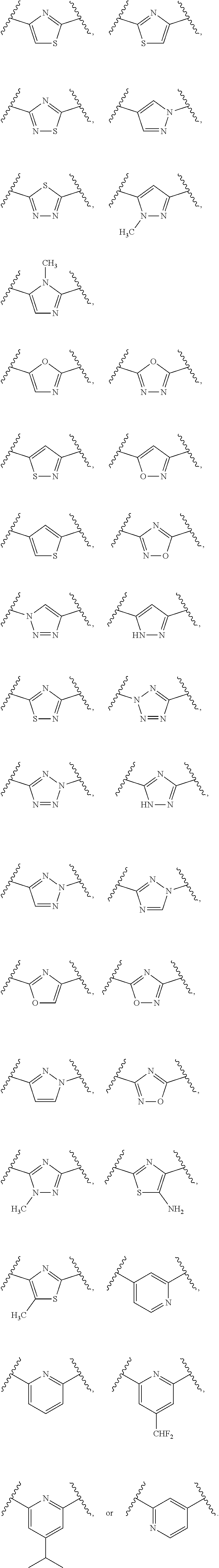

In some embodiments, A is optionally substituted C₁-C₄ heteroalkylene. In some embodiments, A is:

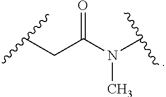 [SMILES: *CC(=O)N(*)C]

In some embodiments, A is optionally substituted 3 to 6-membered heterocycloalkylene. In some embodiments, A is:

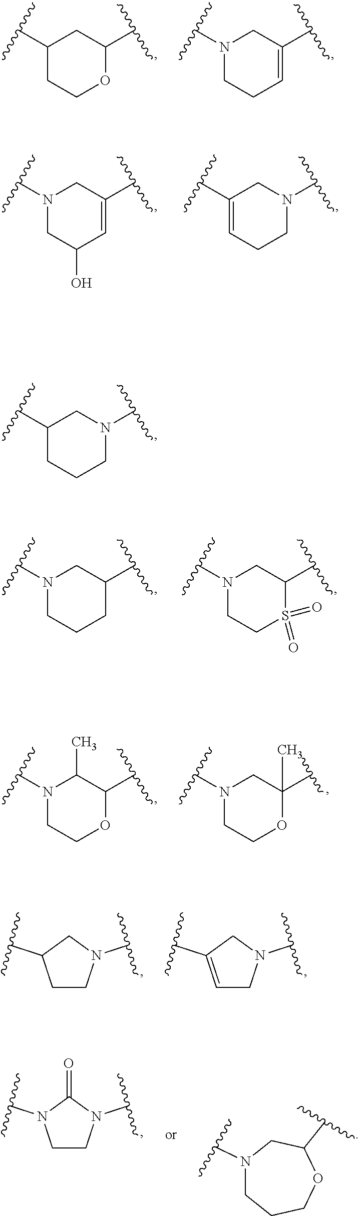

In some embodiments, A is

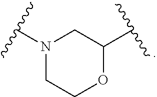 [SMILES: *C1CN(*)CCO1]

In some embodiments of a compound of the present invention, B is —CHR⁹—. In some embodiments, R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl. In some embodiments, R⁹ is:

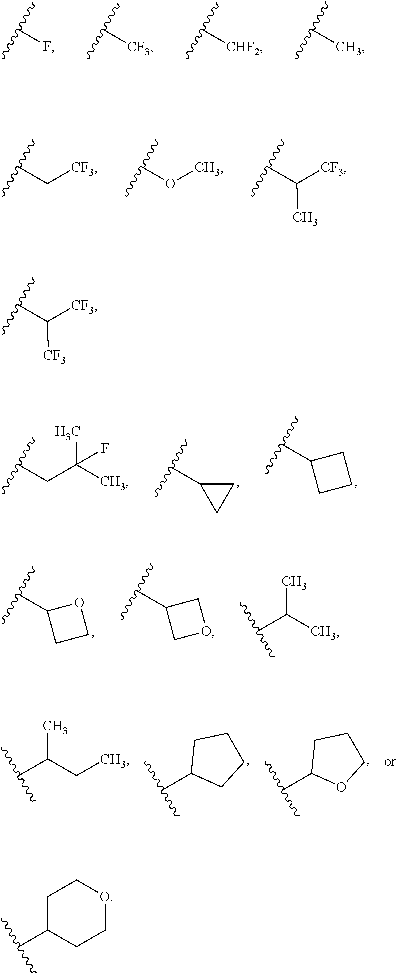

In some embodiments, R⁹ is:

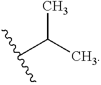 [SMILES: *C(C)C]

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a compound of the present invention, B is optionally substituted 6-membered arylene. In some embodiments, B is 6-membered arylene. In some embodiments, B is:

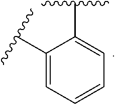 [SMILES: *c1ccccc1*]

In some embodiments of a compound of the present invention, R⁷ is methyl.

In some embodiments of a compound of the present invention, R⁸ is methyl.

In some embodiments, R²¹ is hydrogen.

In some embodiments of a compound of the present invention, the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A². In some embodiments, the linker is acyclic. In some embodiments, linker has the structure of Formula IIa:

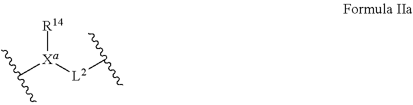

wherein X^{a} is absent or N;

R¹⁴ is absent, hydrogen or optionally substituted C₁-C₆ alkyl; and

L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

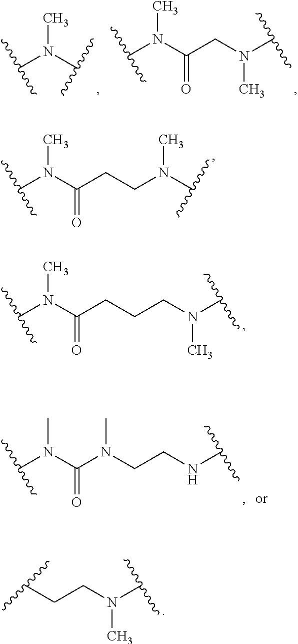

In some embodiments, the linker is or comprises a cyclic moiety. In some embodiments, the linker has the structure of Formula IIb:

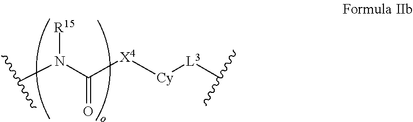

wherein o is 0 or 1;

R¹⁵ is hydrogen or optionally substituted C₁-C₆ alkyl, optionally substituted 3 to 8-membered cycloalkylene, or optionally substituted 3 to 8-membered heterocycloalkylene;

X⁴ is absent, optionally substituted C₁-C₄ alkylene, O, NCH₃, or optionally substituted C₁-C₄ heteroalkylene;

Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —SO₂—, optionally substituted C₁-C₄ alkylene or optionally substituted C₁-C₄ heteroalkylene.

In some embodiments, the linker has the structure of Formula IIb-1:

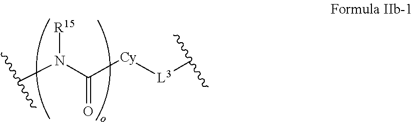

wherein o is 0 or 1;

R¹⁵ is hydrogen or optionally substituted C₁-C₆ alkyl, optionally substituted 3 to 8-membered cycloalkylene, or optionally substituted 3 to 8-membered heterocycloalkylene; Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —SO₂—, optionally substituted C₁-C₄ alkylene or optionally substituted C₁-C₄ heteroalkylene.

In some embodiments, the linker has the structure of Formula IIc:

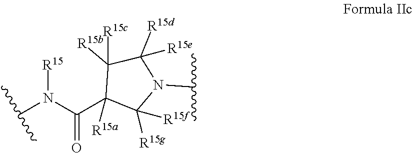

wherein R¹⁵ is hydrogen, optionally substituted C₁-C₆ alkyl, optionally substituted 3 to 8-membered cycloalkylene, or optionally substituted 3 to 8-membered heterocycloalkylene; and

R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

In some embodiments, the linker has the structure:

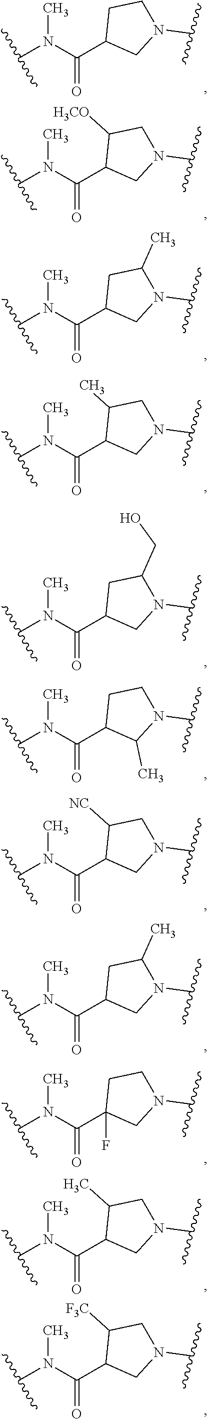

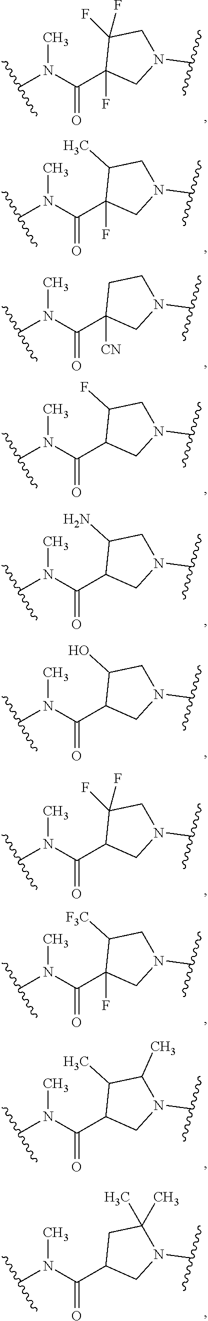

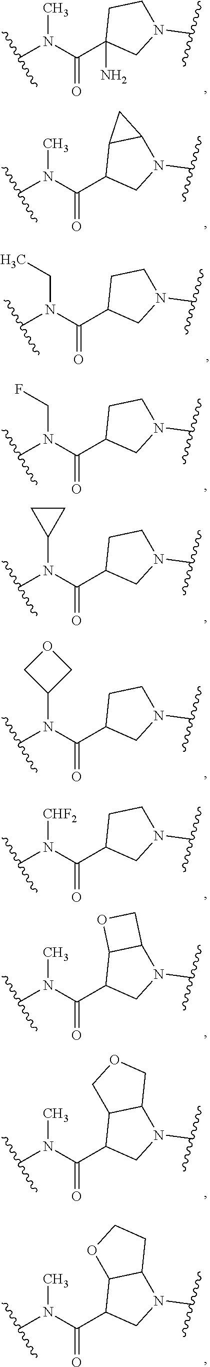

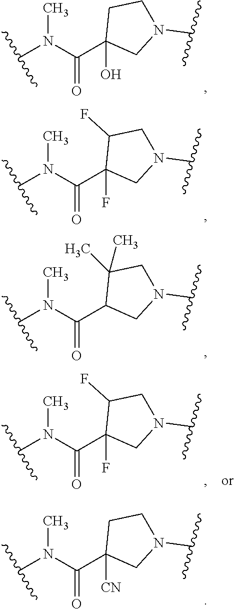

In some embodiments, the linker has the structure:

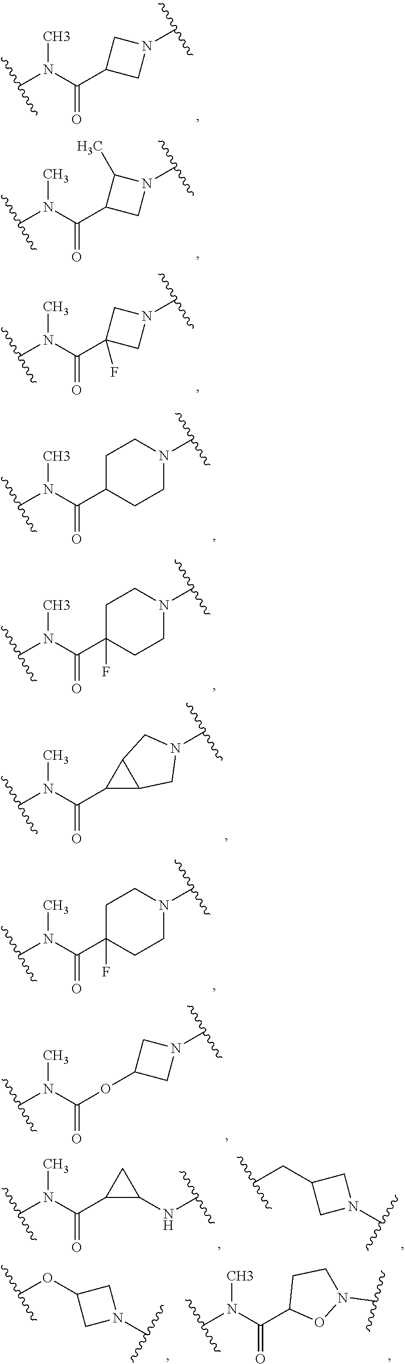

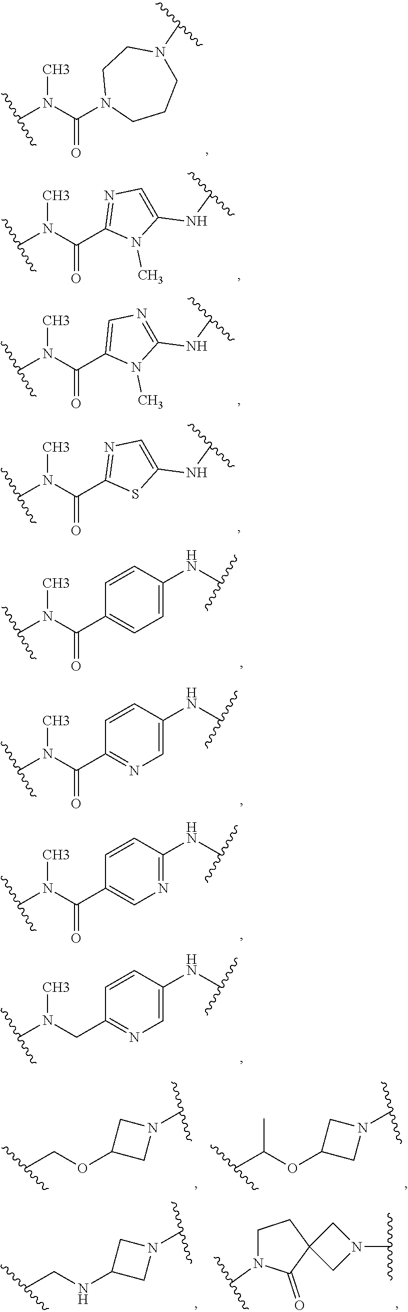

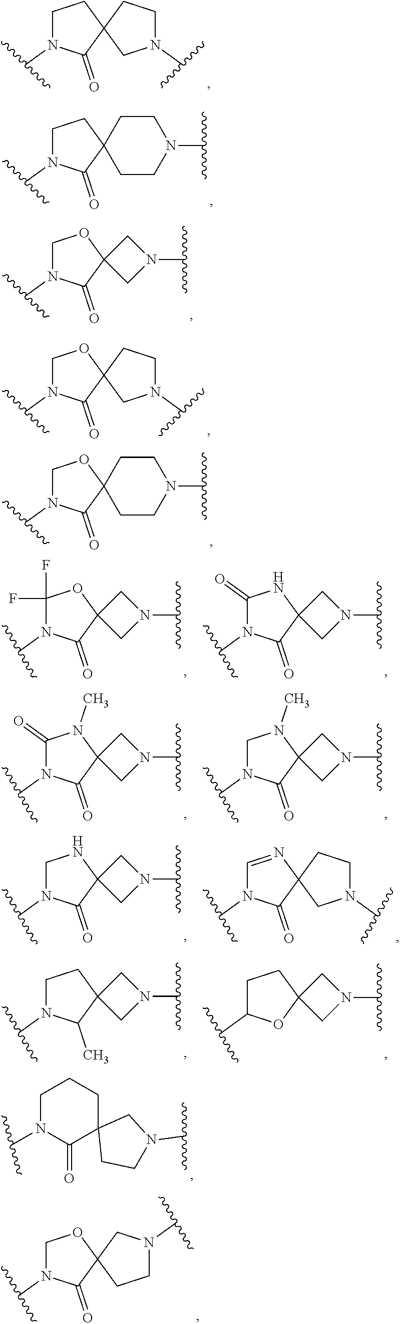

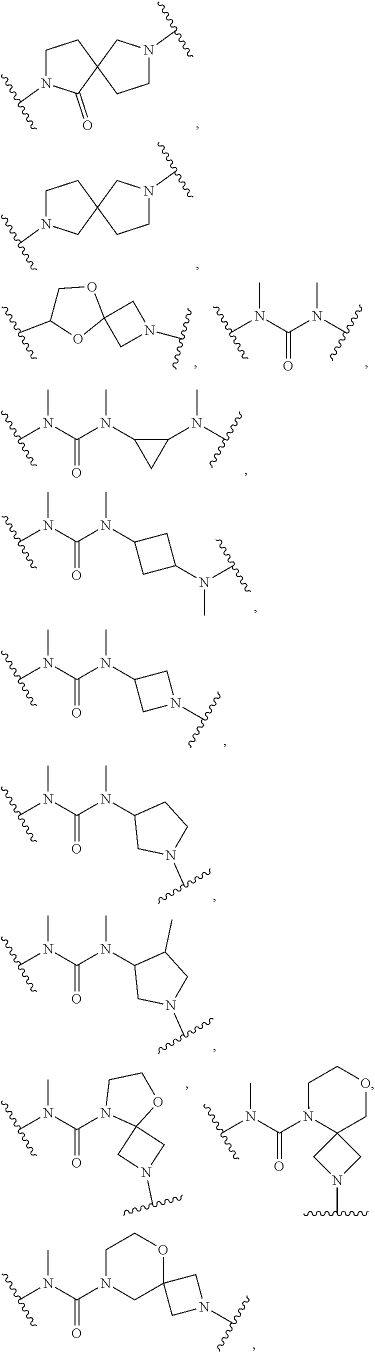

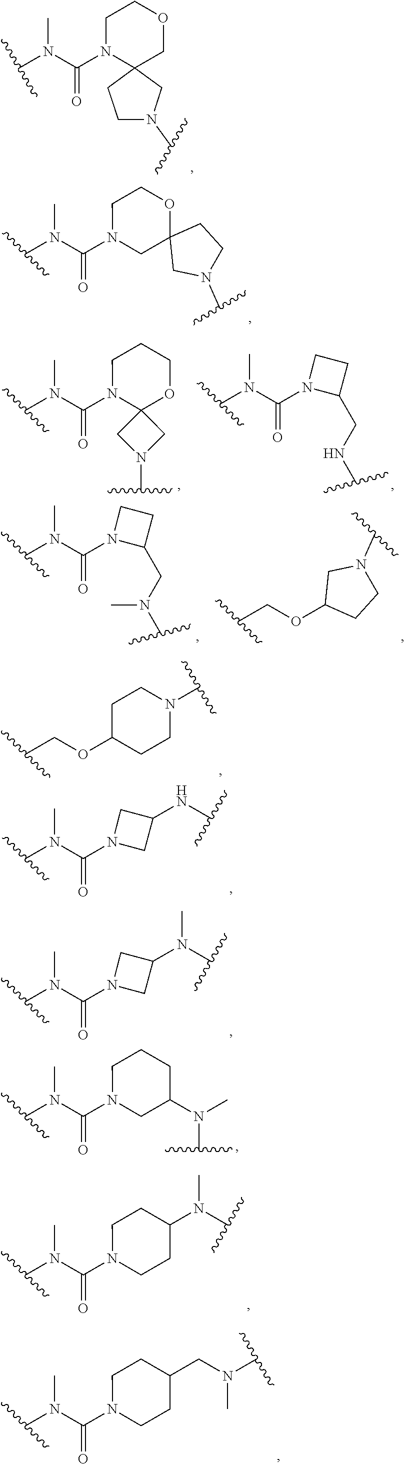

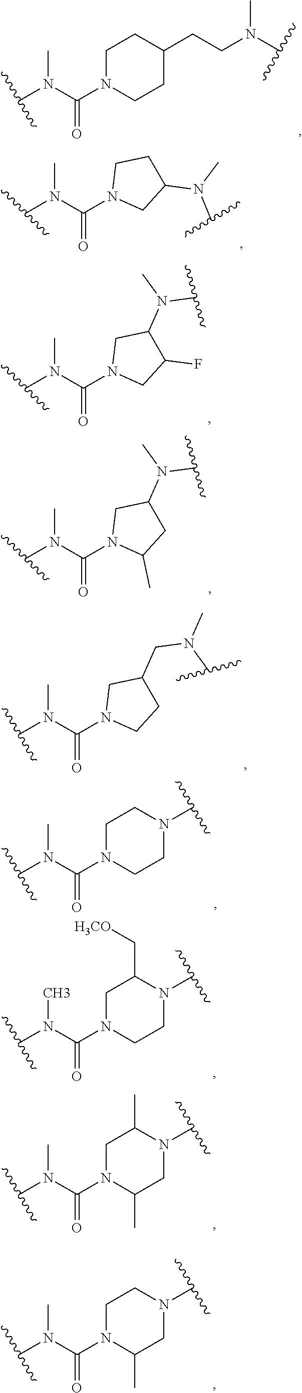

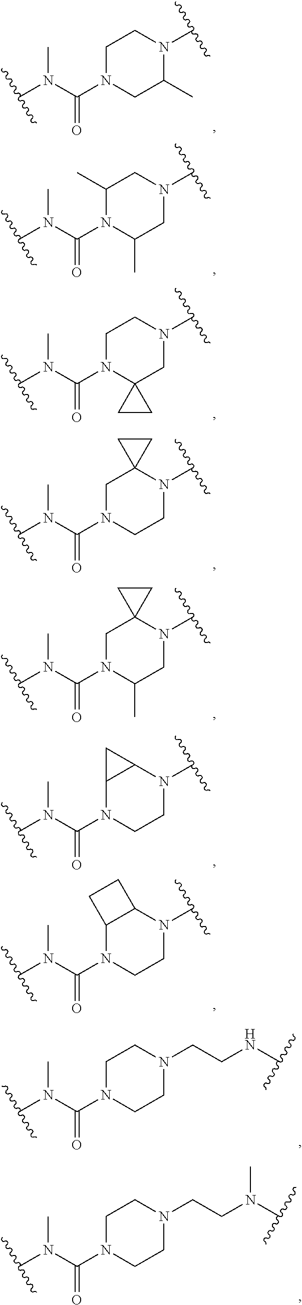

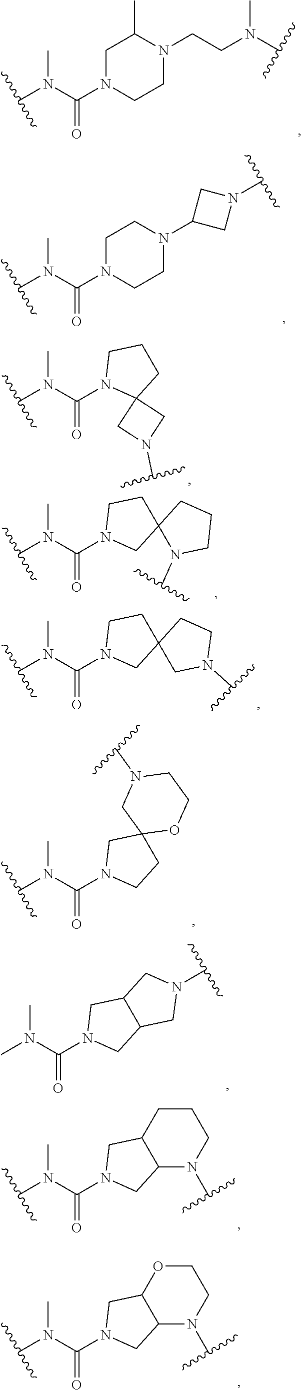

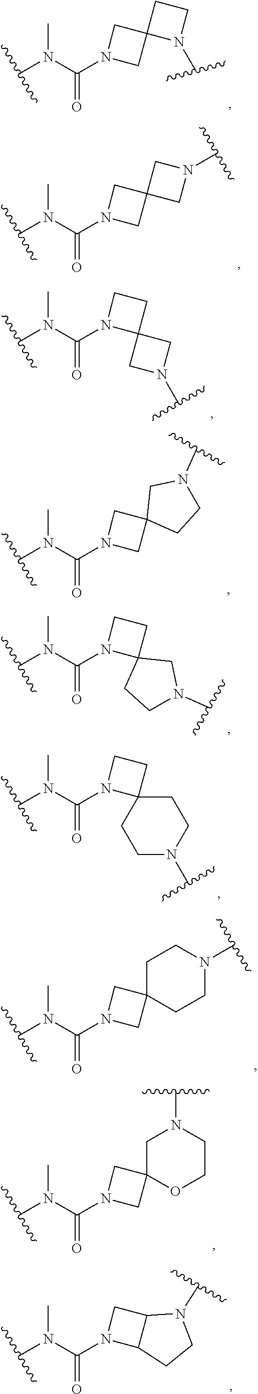

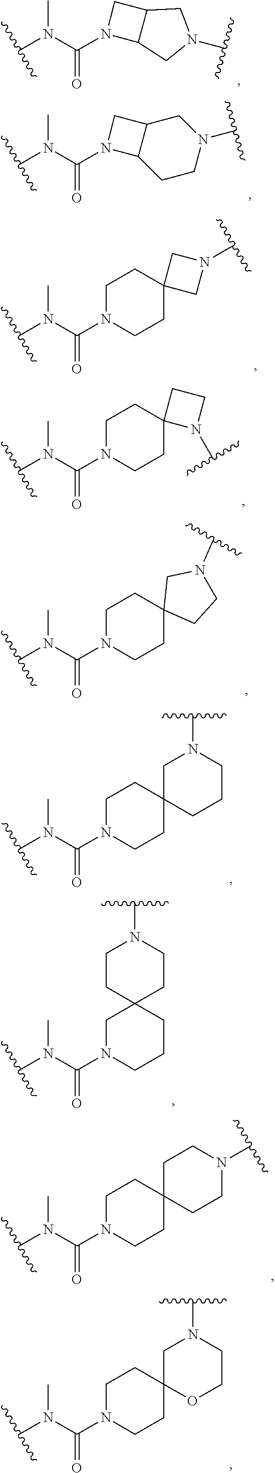

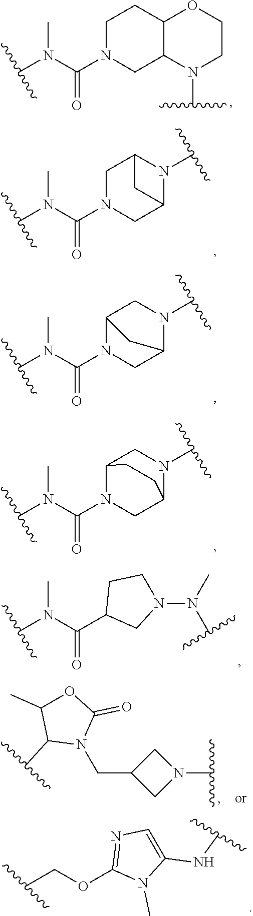

In some embodiments, the linker has the structure

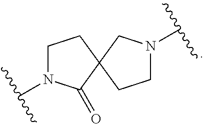 [SMILES: *N1CCC2(CCN(*)C2=O)C1]

In some embodiments, the linker has the structure

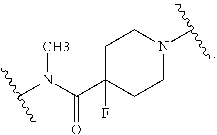 [SMILES: *N1CCC(F)(C(=O)N(*)C)CC1]

In some embodiments of a compound of the present invention, W is a cross-linking group comprising a vinyl ketone. In some embodiments, W has the structure of Formula IIIa:

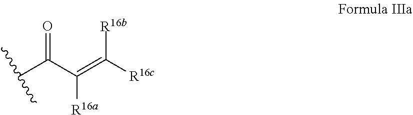

wherein R^{16a}, R^{16b}, and R^{16c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

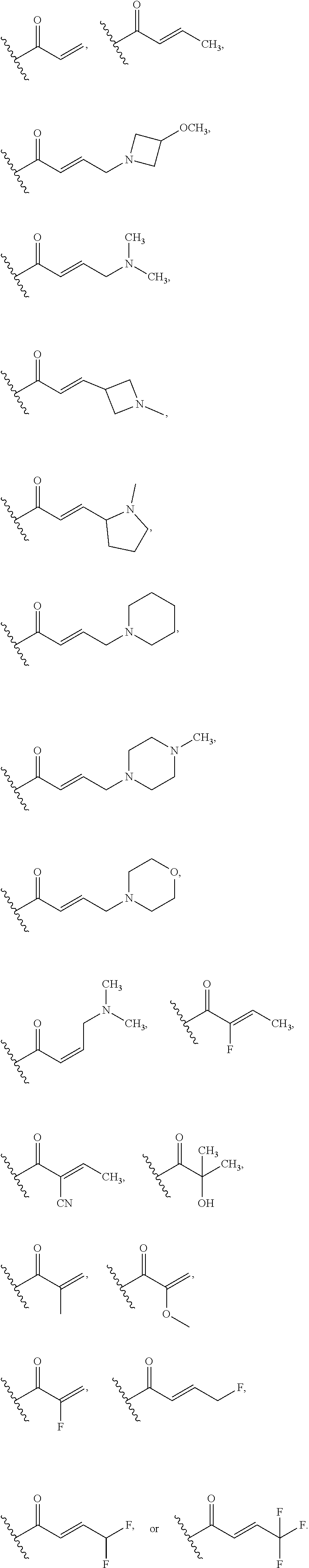

In some embodiments, W is a cross-linking group comprising an ynone. In some embodiments, as the structure of Formula IIIb:

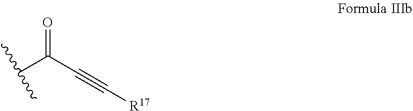

wherein R¹⁷ is hydrogen, —C₁-C₃alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

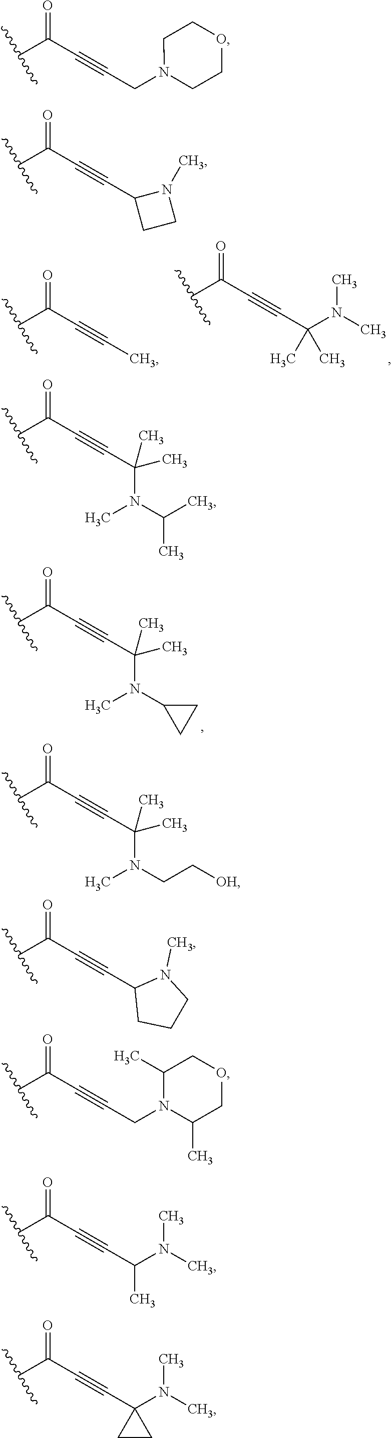

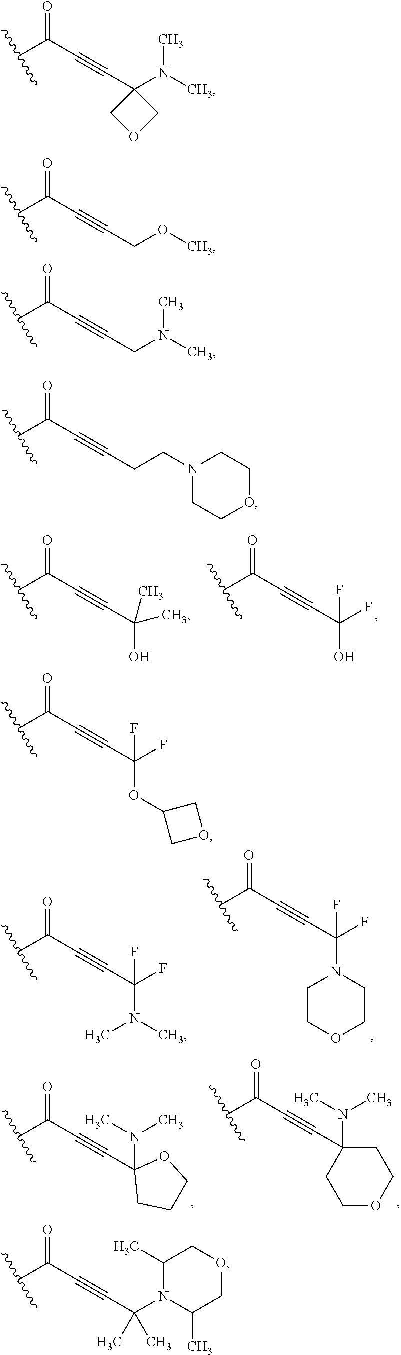

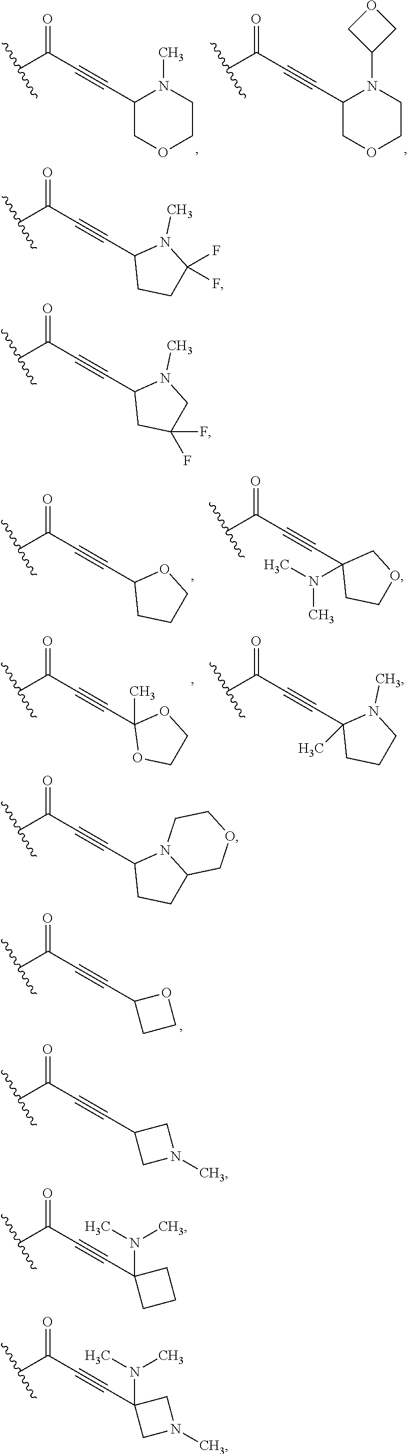

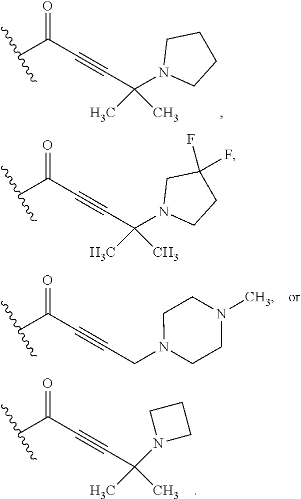

In some embodiments, W is

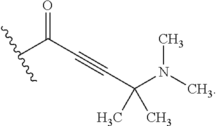 [SMILES: *C(=O)C#CC(C)(C)N(C)C]

In some embodiments, W is a cross-linking group comprising a vinyl sulfone. In some embodiments, W has the structure of Formula IIIc:

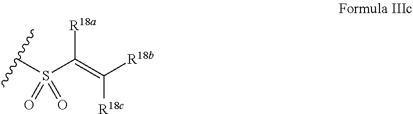

wherein R^{18a}, R^{18b}, and R^{18c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

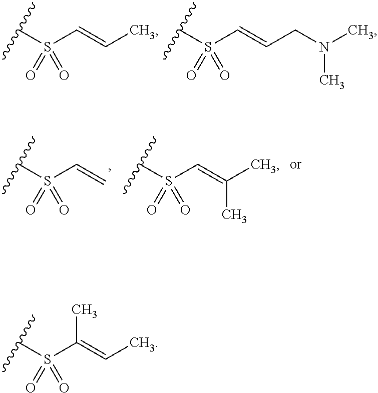

In some embodiments, W is a cross-linking group comprising an alkynyl sulfone. In some embodiments, W has the structure of Formula IIId:

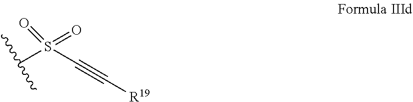

wherein R¹⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is

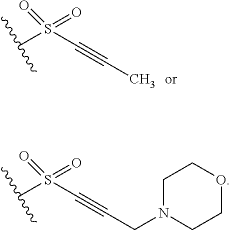

In some embodiments, W has the structure of Formula IIIe:

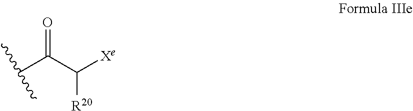

wherein X^{e} is a halogen; and

R²⁰ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is haloacetal. In some embodiments, W is not haloacetal.

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| A1 | 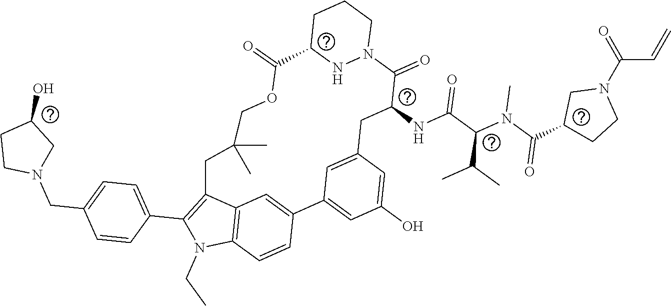 [SMILES: C=CC(=O)N1CC[C@H](C(=O)N(C)[C@H](C(=O)N[C@H]2Cc3cc(O)cc(c3)-c3ccc4c(c3)c(c(-c3ccc(CN5CC[C@@H](O)C5)cc3)n4CC)CC(C)(C)COC(=O)[C@@H]3CCCN(N3)C2=O)C(C)C)C1] |
| A2 | 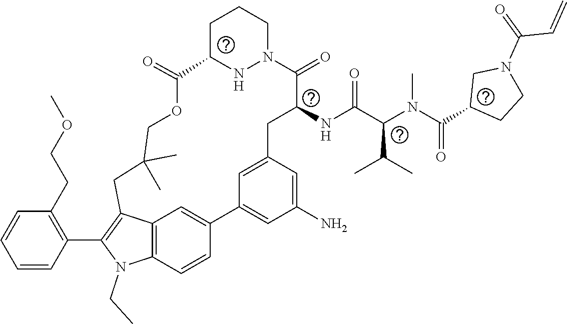 [SMILES: C=CC(=O)N1CC[C@H](C(=O)N(C)[C@H](C(=O)N[C@H]2Cc3cc(N)cc(c3)-c3ccc4c(c3)c(c(-c3ccccc3CCOC)n4CC)CC(C)(C)COC(=O)[C@@H]3CCCN(N3)C2=O)C(C)C)C1] |
| A3 | 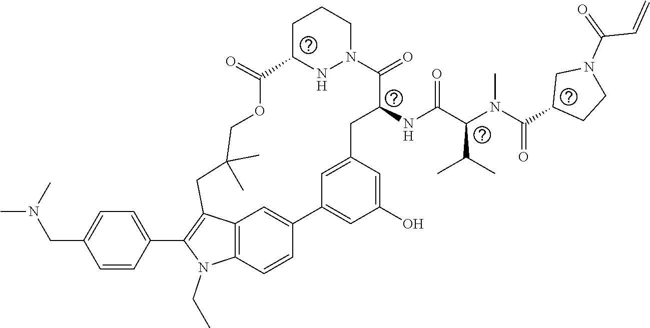 [SMILES: C=CC(=O)N1CC[C@H](C(=O)N(C)[C@H](C(=O)N[C@H]2Cc3cc(O)cc(c3)-c3ccc4c(c3)c(c(-c3ccc(CN(C)C)cc3)n4CC)CC(C)(C)COC(=O)[C@@H]3CCCN(N3)C2=O)C(C)C)C1] |
| A4 | 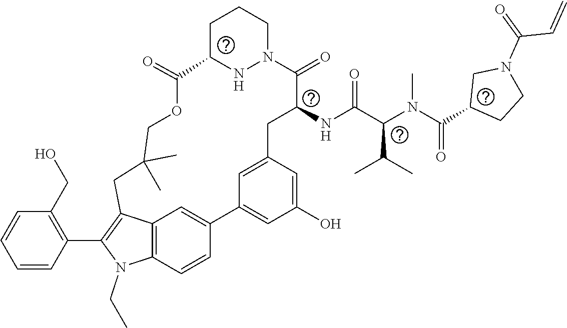 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CO)n4CC)CC(C)(C)COC(=O)[C@@H]3CCCN(N3)C2=O)C(C)C)C1] |
| A5 | 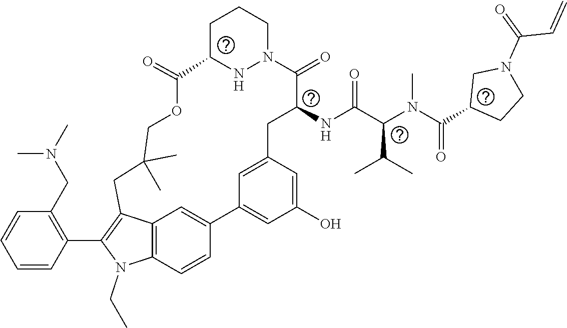 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CN(C)C)n4CC)CC(C)(C)COC(=O)[C@@H]3CCCN(N3)C2=O)C(C)C)C1] |
| A6 | 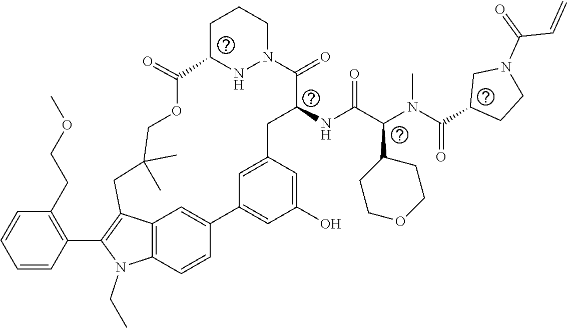 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C2CCOCC2)C1] |
| A7 | 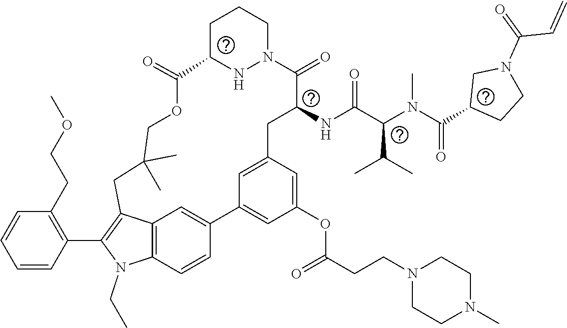 [SMILES: C=CC(=O)N1CC[C@H](C(=O)N(C)[C@H](C(=O)N[C@H]2Cc3cc(OC(=O)CCN4CCN(C)CC4)cc(c3)-c3ccc4c(c3)c(c(-c3ccccc3CCOC)n4CC)CC(C)(C)COC(=O)[C@@H]3CCCN(N3)C2=O)C(C)C)C1] |
| A8 | 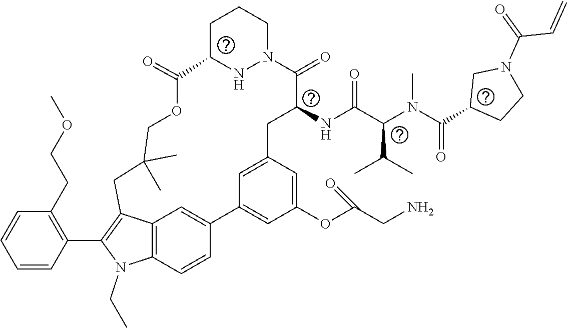 [SMILES: C=CC(=O)N1CC[C@H](C(=O)N(C)[C@H](C(=O)N[C@H]2Cc3cc(OC(=O)CN)cc(c3)-c3ccc4c(c3)c(c(-c3ccccc3CCOC)n4CC)CC(C)(C)COC(=O)[C@@H]3CCCN(N3)C2=O)C(C)C)C1] |
| A9 | 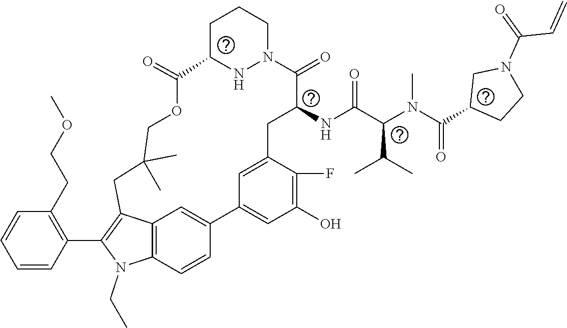 [SMILES: C=CC(=O)N1CC[C@H](C(=O)N(C)[C@H](C(=O)N[C@H]2Cc3cc(cc(O)c3F)-c3ccc4c(c3)c(c(-c3ccccc3CCOC)n4CC)CC(C)(C)COC(=O)[C@@H]3CCCN(N3)C2=O)C(C)C)C1] |
| A10 | 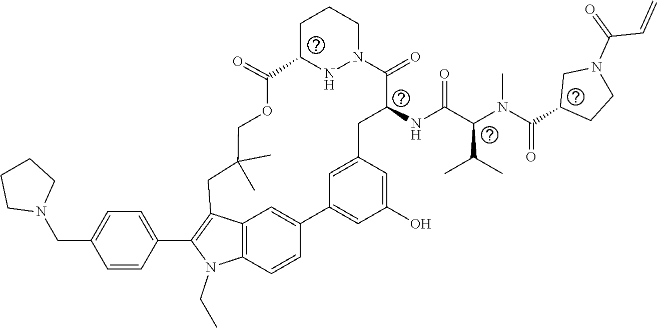 [SMILES: C=CC(=O)N1CC[C@H](C(=O)N(C)[C@H](C(=O)N[C@H]2Cc3cc(O)cc(c3)-c3ccc4c(c3)c(c(-c3ccc(CN5CCCC5)cc3)n4CC)CC(C)(C)COC(=O)[C@@H]3CCCN(N3)C2=O)C(C)C)C1] |
| A11 | 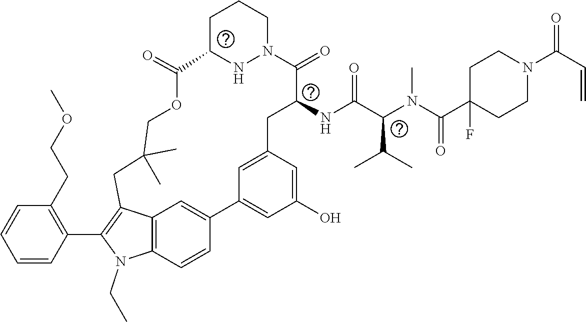 [SMILES: C=CC(=O)N1CCC(F)(C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)CC1] |
| A12 | 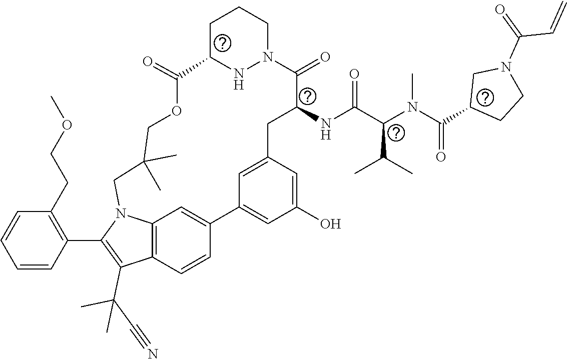 [SMILES: C=CC(=O)N1CC[C@H](C(=O)N(C)[C@H](C(=O)N[C@H]2Cc3cc(O)cc(c3)-c3ccc4c(C(C)(C)C#N)c(-c5ccccc5CCOC)n(c4c3)CC(C)(C)COC(=O)[C@@H]3CCCN(N3)C2=O)C(C)C)C1] |
| A13 | 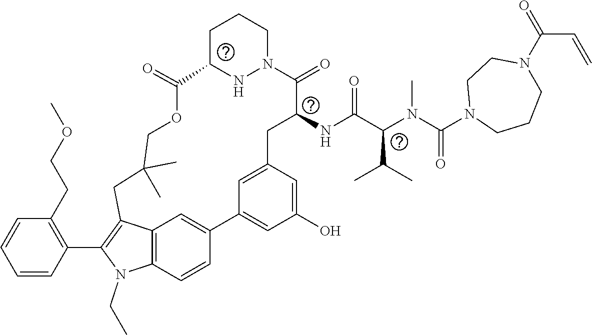 [SMILES: C=CC(=O)N1CCCN(C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)CC1] |
| A14 | 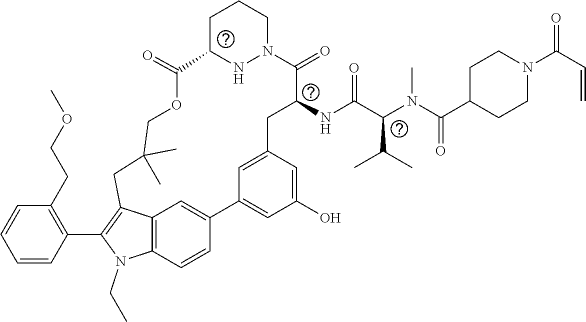 [SMILES: C=CC(=O)N1CCC(C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)CC1] |
| A15 | 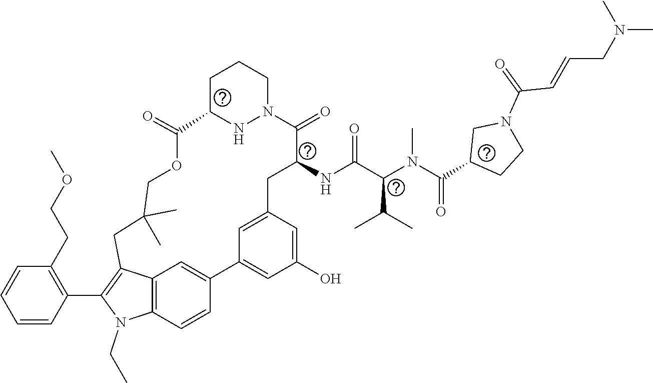 [SMILES: CCn1c(-c2ccccc2CCOC)c2c3cc(ccc31)-c1cc(O)cc(c1)C[C@H](NC(=O)[C@H](C(C)C)N(C)C(=O)[C@H]1CCN(C(=O)/C=C/CN(C)C)C1)C(=O)N1CCC[C@H](N1)C(=O)OCC(C)(C)C2] |
| A16 | 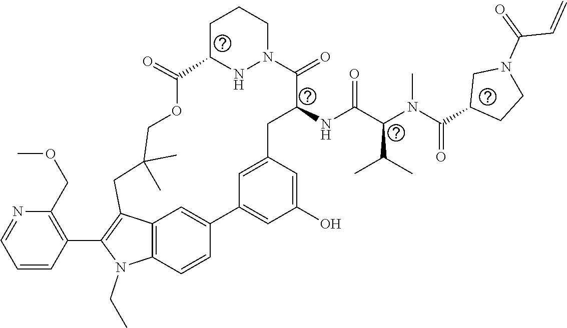 [SMILES: C=CC(=O)N1CC[C@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A17 | 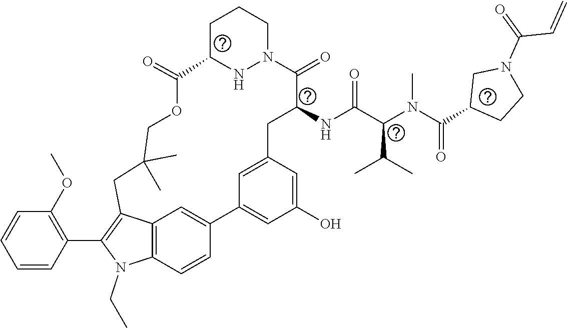 [SMILES: C=CC(=O)N1CC[C@H](C(=O)N(C)[C@H](C(=O)N[C@H]2Cc3cc(O)cc(c3)-c3ccc4c(c3)c(c(-c3ccccc3OC)n4CC)CC(C)(C)COC(=O)[C@@H]3CCCN(N3)C2=O)C(C)C)C1] |
| A18 | 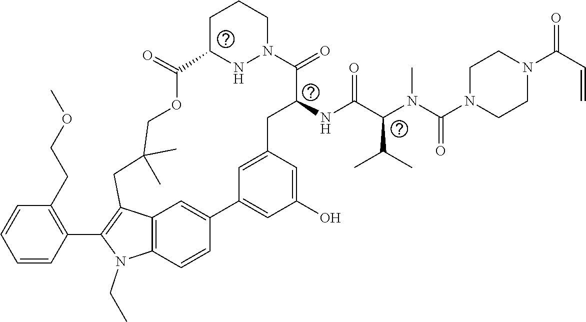 [SMILES: C=CC(=O)N1CCN(C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)CC1] |
| A19 | 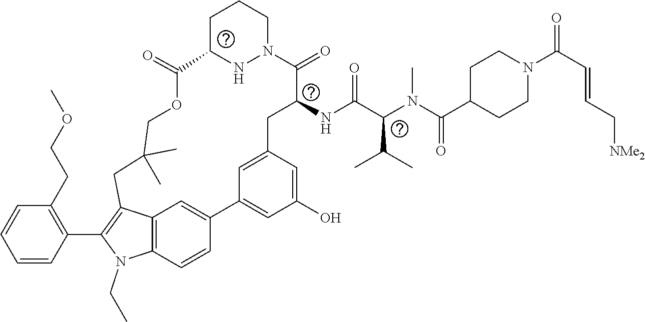 [SMILES: CCn1c(-c2ccccc2CCOC)c2c3cc(ccc31)-c1cc(O)cc(c1)C[C@H](NC(=O)[C@H](C(C)C)N(C)C(=O)C1CCN(C(=O)/C=C/CN(C)C)CC1)C(=O)N1CCC[C@H](N1)C(=O)OCC(C)(C)C2] |
| A20 | 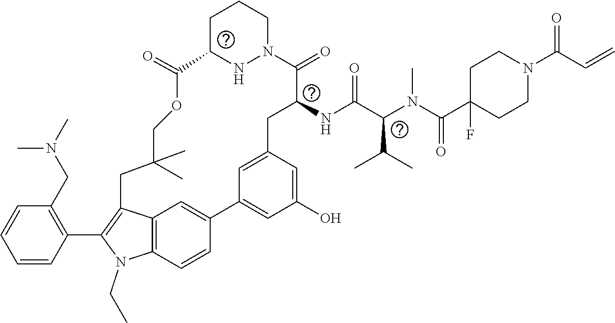 [SMILES: C=CC(=O)N1CCC(F)(C(=O)N(C)[C@H](C(=O)N[C@H]2Cc3cc(O)cc(c3)-c3ccc4c(c3)c(c(-c3ccccc3CN(C)C)n4CC)CC(C)(C)COC(=O)[C@@H]3CCCN(N3)C2=O)C(C)C)CC1] |
| A21 | 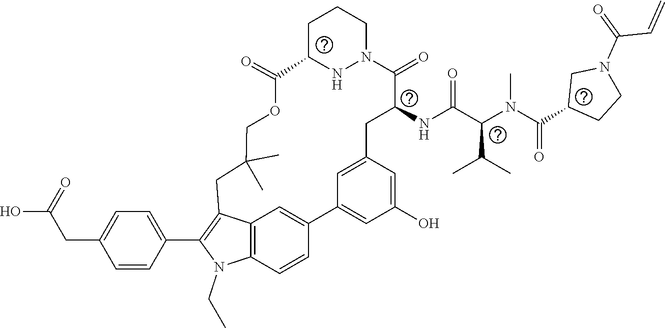 [SMILES: C=CC(=O)N1CC[C@H](C(=O)N(C)[C@H](C(=O)N[C@H]2Cc3cc(O)cc(c3)-c3ccc4c(c3)c(c(-c3ccc(CC(=O)O)cc3)n4CC)CC(C)(C)COC(=O)[C@@H]3CCCN(N3)C2=O)C(C)C)C1] |
| A22 | 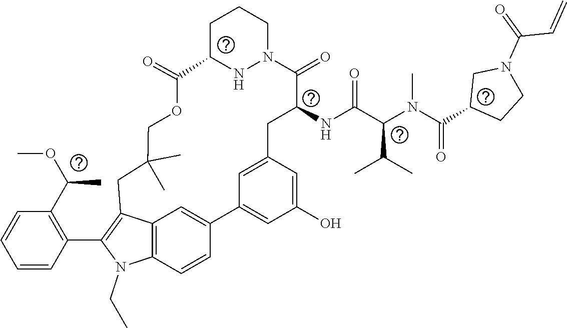 [SMILES: C=CC(=O)N1CC[C@H](C(=O)N(C)[C@H](C(=O)N[C@H]2Cc3cc(O)cc(c3)-c3ccc4c(c3)c(c(-c3ccccc3[C@H](C)OC)n4CC)CC(C)(C)COC(=O)[C@@H]3CCCN(N3)C2=O)C(C)C)C1] |
| A23 | 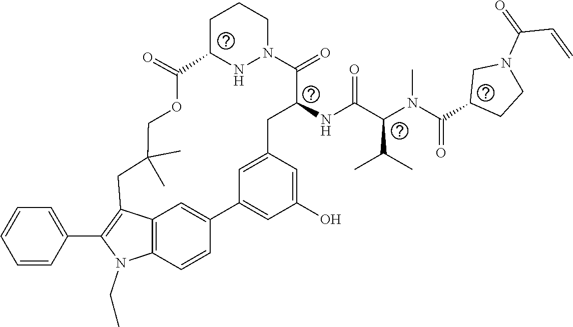 [SMILES: C=CC(=O)N1CC[C@H](C(=O)N(C)[C@H](C(=O)N[C@H]2Cc3cc(O)cc(c3)-c3ccc4c(c3)c(c(-c3ccccc3)n4CC)CC(C)(C)COC(=O)[C@@H]3CCCN(N3)C2=O)C(C)C)C1] |
| A24 | 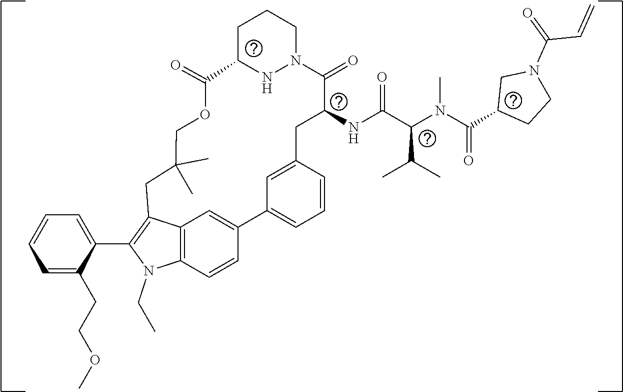 |
| A25 | 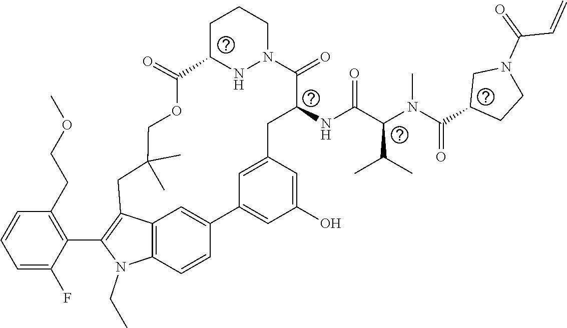 [SMILES: C=CC(=O)N1CC[C@H](C(=O)N(C)[C@H](C(=O)N[C@H]2Cc3cc(O)cc(c3)-c3ccc4c(c3)c(c(-c3c(F)cccc3CCOC)n4CC)CC(C)(C)COC(=O)[C@@H]3CCCN(N3)C2=O)C(C)C)C1] |
| A26 | 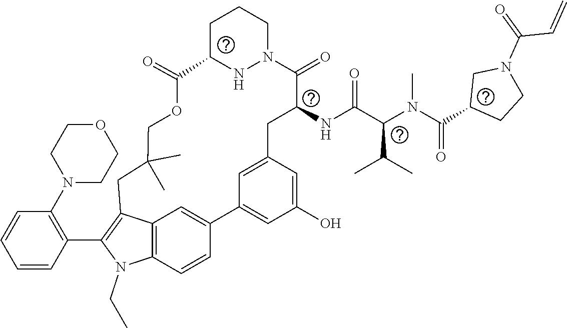 [SMILES: C=CC(=O)N1CC[C@H](C(=O)N(C)[C@H](C(=O)N[C@H]2Cc3cc(O)cc(c3)-c3ccc4c(c3)c(c(-c3ccccc3N3CCOCC3)n4CC)CC(C)(C)COC(=O)[C@@H]3CCCN(N3)C2=O)C(C)C)C1] |
| A27 | 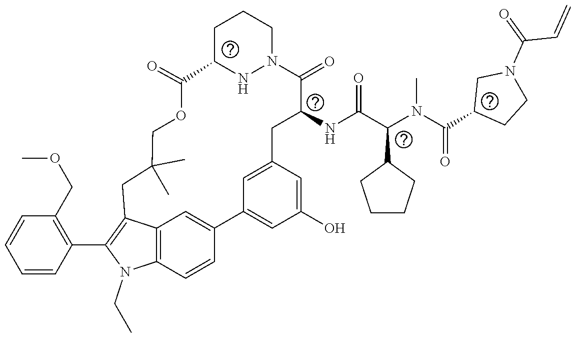 [SMILES: C=CC(=O)N1CC[C@H](C(=O)N(C)[C@H](C(=O)N[C@H]2Cc3cc(O)cc(c3)-c3ccc4c(c3)c(c(-c3ccccc3COC)n4CC)CC(C)(C)COC(=O)[C@@H]3CCCN(N3)C2=O)C2CCCC2)C1] |
| A28 | 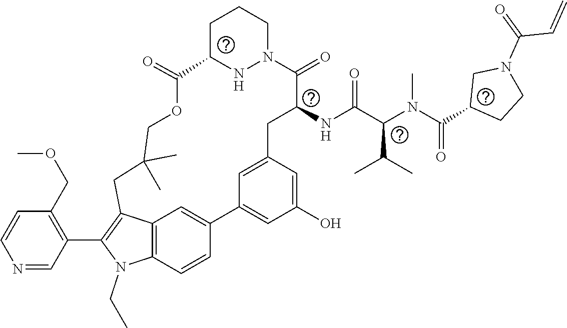 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)C1] |
| A29 | 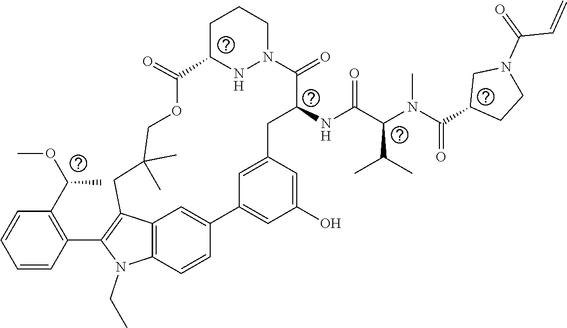 [SMILES: C=CC(=O)N1CC[C@H](C(=O)N(C)[C@H](C(=O)N[C@H]2Cc3cc(O)cc(c3)-c3ccc4c(c3)c(c(-c3ccccc3[C@@H](C)OC)n4CC)CC(C)(C)COC(=O)[C@@H]3CCCN(N3)C2=O)C(C)C)C1] |
| A30 | 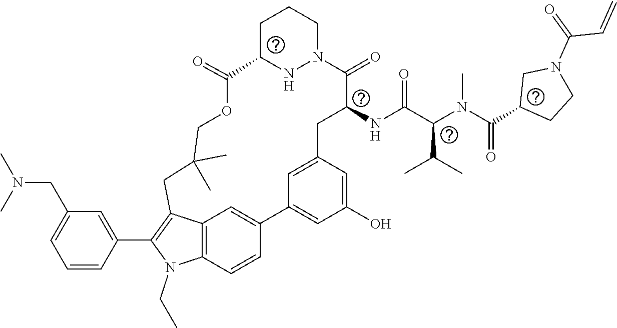 [SMILES: C=CC(=O)N1CC[C@H](C(=O)N(C)[C@H](C(=O)N[C@H]2Cc3cc(O)cc(c3)-c3ccc4c(c3)c(c(-c3cccc(CN(C)C)c3)n4CC)CC(C)(C)COC(=O)[C@@H]3CCCN(N3)C2=O)C(C)C)C1] |
| A31 | 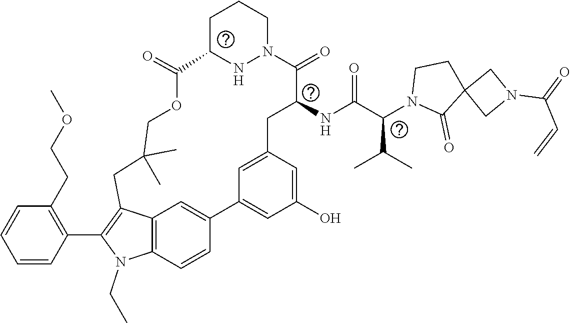 [SMILES: C=CC(=O)N1CC2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| A32 | 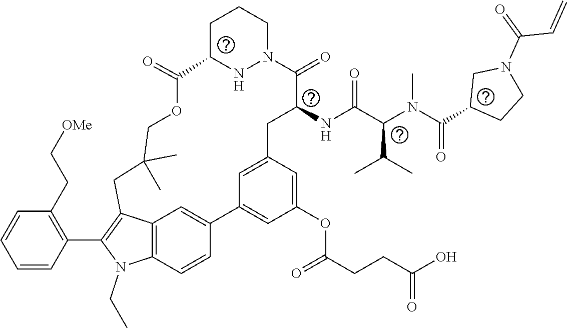 [SMILES: C=CC(=O)N1CC[C@H](C(=O)N(C)[C@H](C(=O)N[C@H]2Cc3cc(OC(=O)CCC(=O)O)cc(c3)-c3ccc4c(c3)c(c(-c3ccccc3CCOC)n4CC)CC(C)(C)COC(=O)[C@@H]3CCCN(N3)C2=O)C(C)C)C1] |
| A33 | 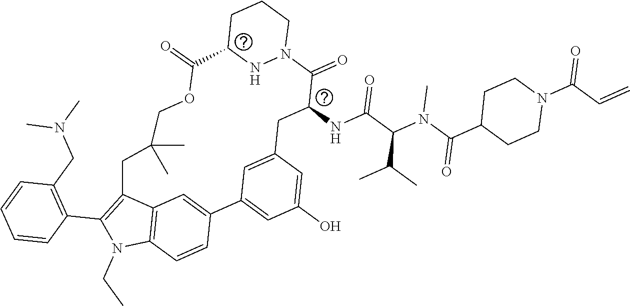 [SMILES: C=CC(=O)N1CCC(C(=O)N(C)[C@H](C(=O)N[C@H]2Cc3cc(O)cc(c3)-c3ccc4c(c3)c(c(-c3ccccc3CN(C)C)n4CC)CC(C)(C)COC(=O)[C@@H]3CCCN(N3)C2=O)C(C)C)CC1] |
| A34 | 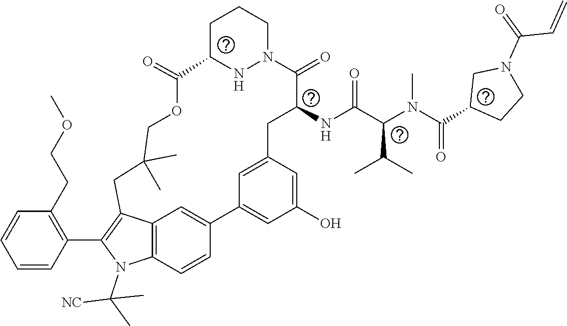 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4C(C)(C)C#N)CC(C)(C)COC(=O)[C@@H]3CCCN(N3)C2=O)C(C)C)C1] |
| A35 | 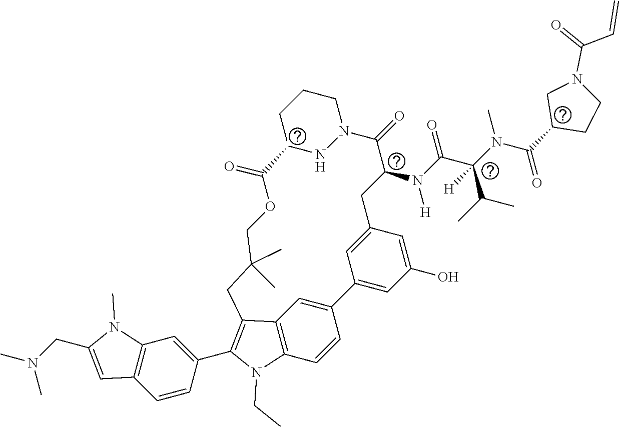 [SMILES: C=CC(=O)N1CC[C@H](C(=O)N(C)[C@H](C(=O)N[C@H]2Cc3cc(O)cc(c3)-c3ccc4c(c3)c(c(-c3ccc5cc(CN(C)C)n(C)c5c3)n4CC)CC(C)(C)COC(=O)[C@@H]3CCCN(N3)C2=O)C(C)C)C1] |
| A36 | 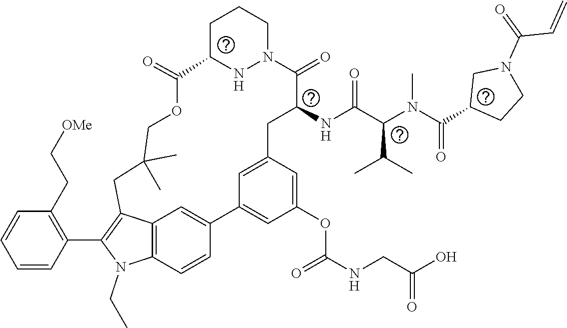 [SMILES: C=CC(=O)N1CC[C@H](C(=O)N(C)[C@H](C(=O)N[C@H]2Cc3cc(OC(=O)NCC(=O)O)cc(c3)-c3ccc4c(c3)c(c(-c3ccccc3CCOC)n4CC)CC(C)(C)COC(=O)[C@@H]3CCCN(N3)C2=O)C(C)C)C1] |
| A37 | 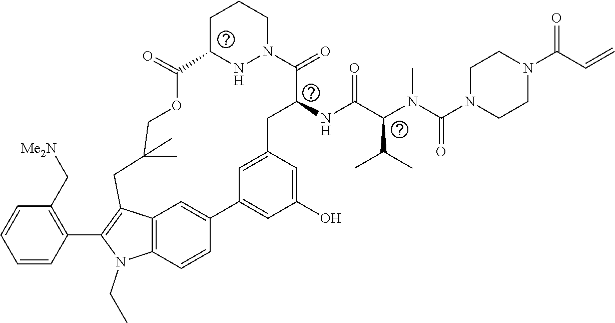 [SMILES: C=CC(=O)N1CCN(C(=O)N(C)[C@H](C(=O)N[C@H]2Cc3cc(O)cc(c3)-c3ccc4c(c3)c(c(-c3ccccc3CN(C)C)n4CC)CC(C)(C)COC(=O)[C@@H]3CCCN(N3)C2=O)C(C)C)CC1] |
| A38 | 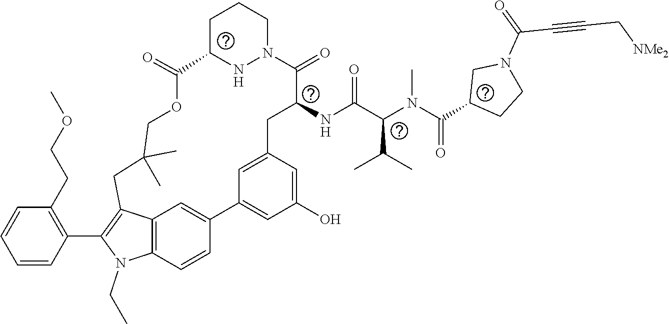 [SMILES: CCn1c(-c2ccccc2CCOC)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A39 | 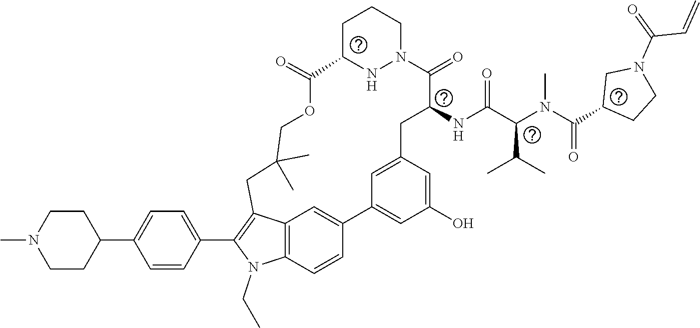 [SMILES: C=CC(=O)N1CC[C@H](C(=O)N(C)[C@H](C(=O)N[C@H]2Cc3cc(O)cc(c3)-c3ccc4c(c3)c(c(-c3ccc(C5CCN(C)CC5)cc3)n4CC)CC(C)(C)COC(=O)[C@@H]3CCCN(N3)C2=O)C(C)C)C1] |
| A40 | 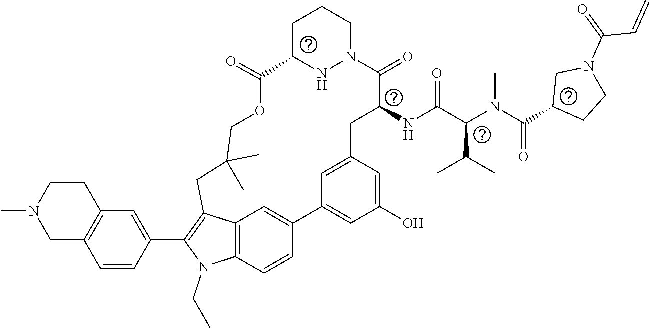 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CCN(C)C5)n4CC)CC(C)(C)COC(=O)[C@@H]3CCCN(N3)C2=O)C(C)C)C1] |
| A41 | 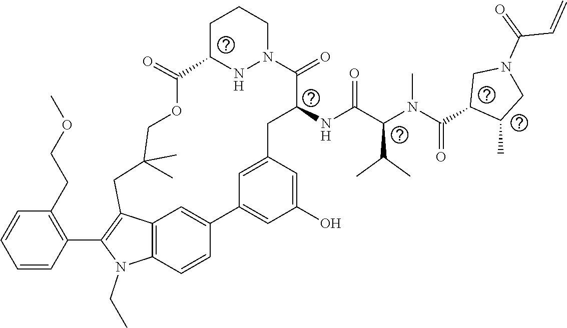 [SMILES: C=CC(=O)N1C[C@H](C)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| A42 | 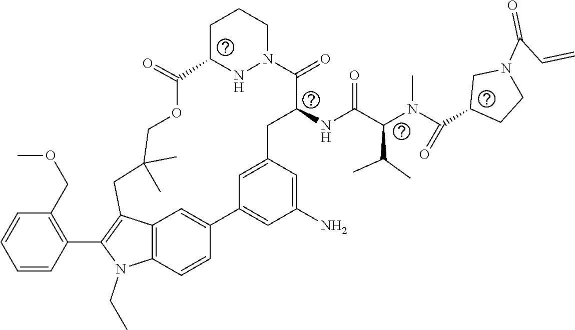 [SMILES: C=CC(=O)N1CC[C@H](C(=O)N(C)[C@H](C(=O)N[C@H]2Cc3cc(N)cc(c3)-c3ccc4c(c3)c(c(-c3ccccc3COC)n4CC)CC(C)(C)COC(=O)[C@@H]3CCCN(N3)C2=O)C(C)C)C1] |
| A43 | 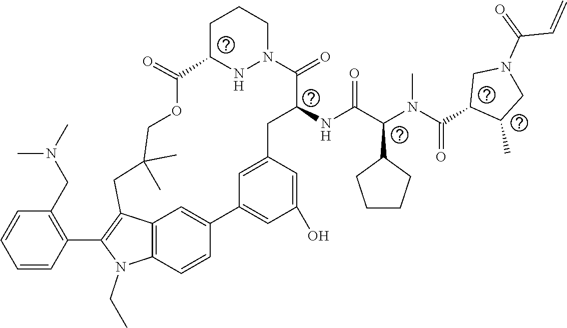 [SMILES: C=CC(=O)N1C[C@H](C)[C@H](C(=O)N(C)[C@H](C(=O)N[C@H]2Cc3cc(O)cc(c3)-c3ccc4c(c3)c(c(-c3ccccc3CN(C)C)n4CC)CC(C)(C)COC(=O)[C@@H]3CCCN(N3)C2=O)C2CCCC2)C1] |
| A44 | 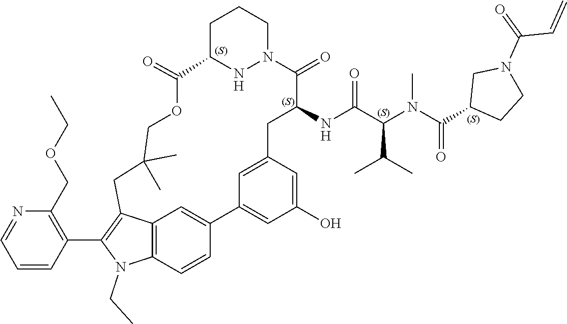 |
| A45 | 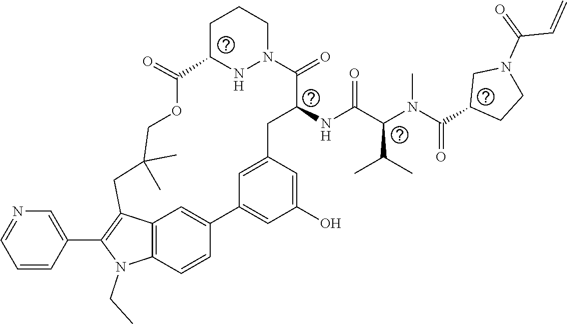 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4CC)CC(C)(C)COC(=O)[C@@H]3CCCN(N3)C2=O)C(C)C)C1] |
| A46 | 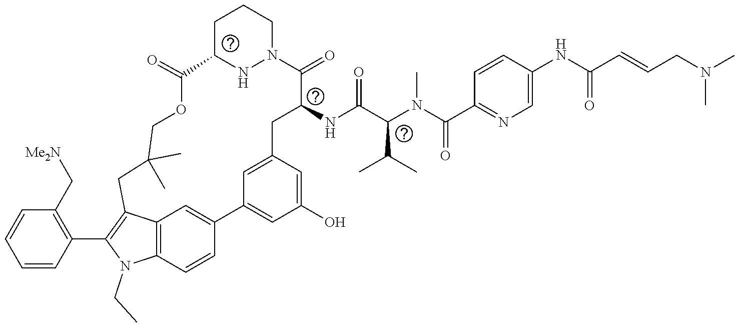 [SMILES: CCn1c(-c2ccccc2CN(C)C)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| A47 | 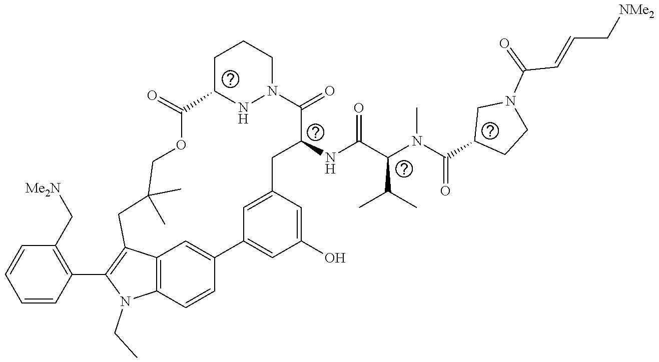 [SMILES: CCn1c(-c2ccccc2CN(C)C)c2c3cc(ccc31)-c1cc(O)cc(c1)C[C@H](NC(=O)[C@H](C(C)C)N(C)C(=O)[C@H]1CCN(C(=O)/C=C/CN(C)C)C1)C(=O)N1CCC[C@H](N1)C(=O)OCC(C)(C)C2] |
| A48 | 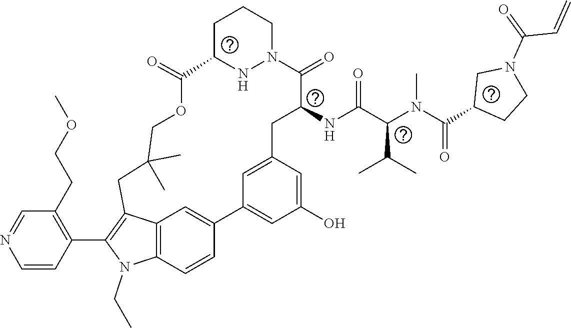 [SMILES: C=CC(=O)N1CC[C@H](C(=O)N(C)[C@H](C(=O)N[C@H]2Cc3cc(O)cc(c3)-c3ccc4c(c3)c(c(-c3ccncc3CCOC)n4CC)CC(C)(C)COC(=O)[C@@H]3CCCN(N3)C2=O)C(C)C)C1] |
| A49 | 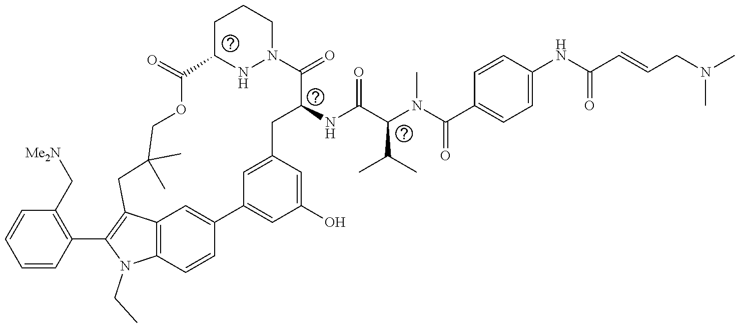 [SMILES: CCn1c(-c2ccccc2CN(C)C)c2c3cc(ccc31)-c1cc(O)cc(c1)C[C@H](NC(=O)[C@H](C(C)C)N(C)C(=O)c1ccc(NC(=O)/C=C/CN(C)C)cc1)C(=O)N1CCC[C@H](N1)C(=O)OCC(C)(C)C2] |
| A50 | 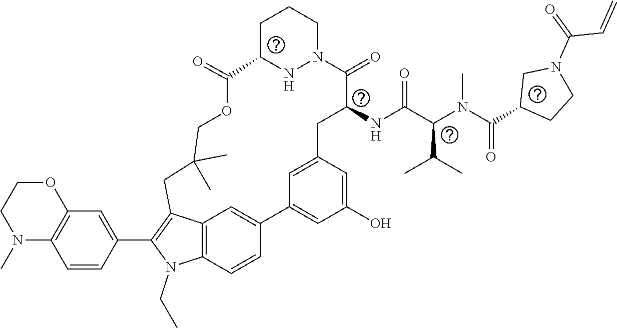 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)OCCN5C)n4CC)CC(C)(C)COC(=O)[C@@H]3CCCN(N3)C2=O)C(C)C)C1] |
| A51 | 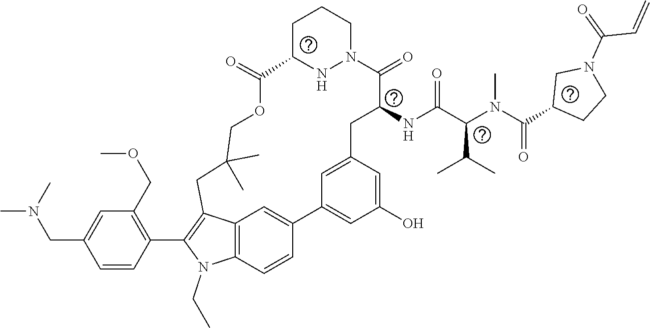 [SMILES: C=CC(=O)N1CC[C@H](C(=O)N(C)[C@H](C(=O)N[C@H]2Cc3cc(O)cc(c3)-c3ccc4c(c3)c(c(-c3ccc(CN(C)C)cc3COC)n4CC)CC(C)(C)COC(=O)[C@@H]3CCCN(N3)C2=O)C(C)C)C1] |
| A52 | 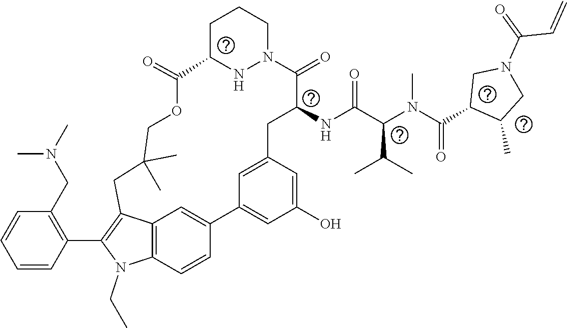 [SMILES: C=CC(=O)N1C[C@H](C)[C@H](C(=O)N(C)[C@H](C(=O)N[C@H]2Cc3cc(O)cc(c3)-c3ccc4c(c3)c(c(-c3ccccc3CN(C)C)n4CC)CC(C)(C)COC(=O)[C@@H]3CCCN(N3)C2=O)C(C)C)C1] |
| A53 | 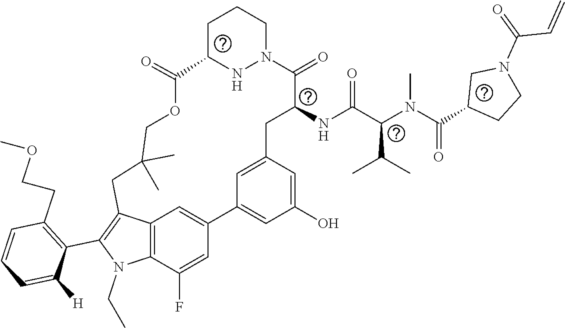 |
| A54 | 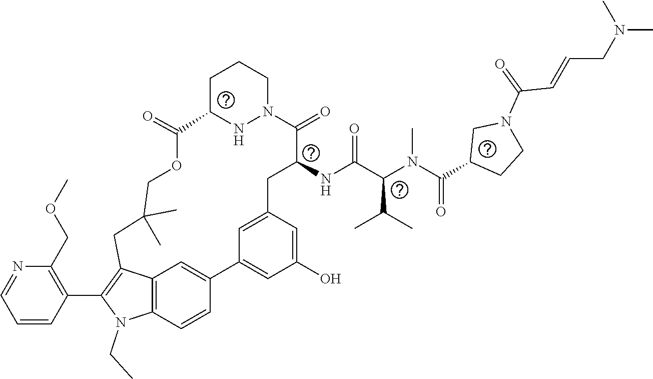 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)/C=C/CN(C)C)C1)C(=O)N1CCC[C@H](N1)C(=O)OCC(C)(C)C2] |
| A55 | 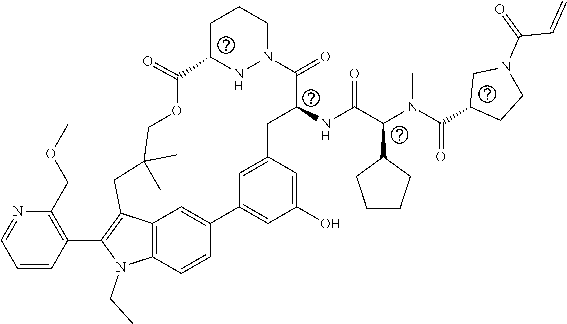 [SMILES: C=CC(=O)N1CC[C@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C2CCCC2)C1] |
| A56 | 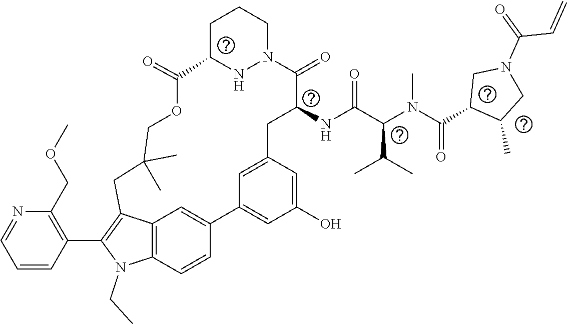 [SMILES: C=CC(=O)N1C[C@H](C)[C@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A57 | 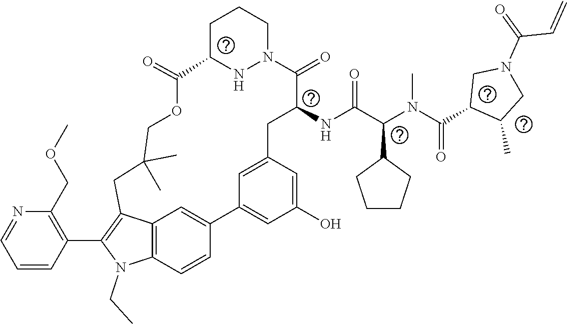 [SMILES: C=CC(=O)N1C[C@H](C)[C@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C2CCCC2)C1] |
| A58 | 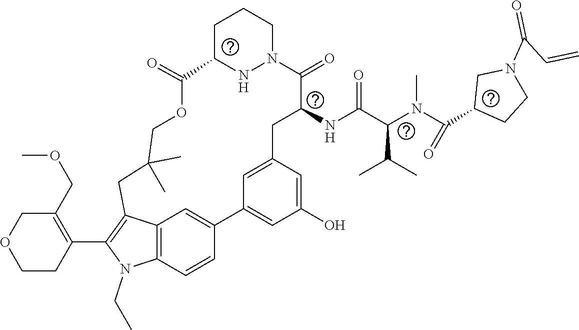 [SMILES: C=CC(=O)N1CC[C@H](C(=O)N(C)[C@H](C(=O)N[C@H]2Cc3cc(O)cc(c3)-c3ccc4c(c3)c(c(C3=C(COC)COCC3)n4CC)CC(C)(C)COC(=O)[C@@H]3CCCN(N3)C2=O)C(C)C)C1] |
| A59 | 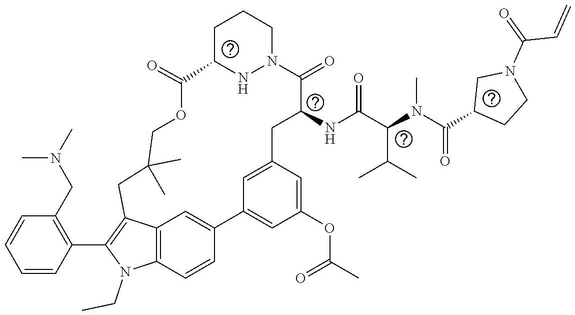 [SMILES: C=CC(=O)N1CC[C@H](C(=O)N(C)[C@H](C(=O)N[C@H]2Cc3cc(OC(C)=O)cc(c3)-c3ccc4c(c3)c(c(-c3ccccc3CN(C)C)n4CC)CC(C)(C)COC(=O)[C@@H]3CCCN(N3)C2=O)C(C)C)C1] |
| A60 | 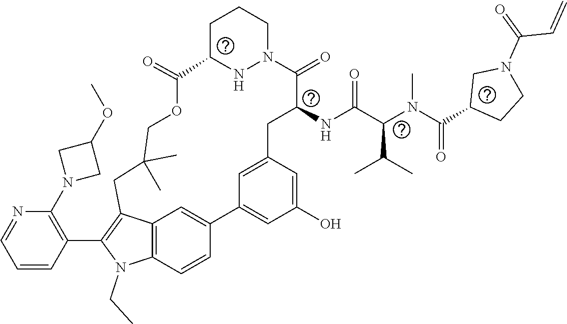 [SMILES: C=CC(=O)N1CC[C@H](C(=O)N(C)[C@H](C(=O)N[C@H]2Cc3cc(O)cc(c3)-c3ccc4c(c3)c(c(-c3cccnc3N3CC(OC)C3)n4CC)CC(C)(C)COC(=O)[C@@H]3CCCN(N3)C2=O)C(C)C)C1] |
| A61 | 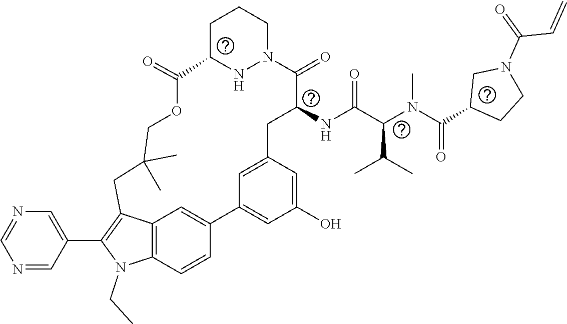 [SMILES: C=CC(=O)N1CC[C@H](C(=O)N(C)[C@H](C(=O)N[C@H]2Cc3cc(O)cc(c3)-c3ccc4c(c3)c(c(-c3cncnc3)n4CC)CC(C)(C)COC(=O)[C@@H]3CCCN(N3)C2=O)C(C)C)C1] |
| A62 | 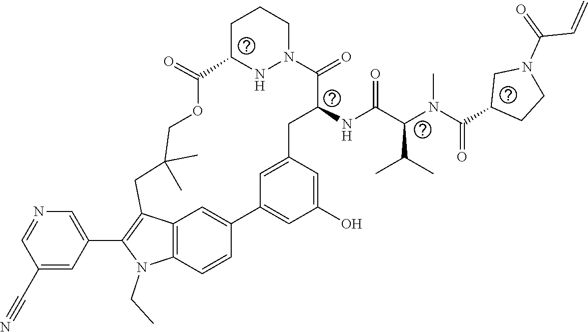 [SMILES: C=CC(=O)N1CC[C@H](C(=O)N(C)[C@H](C(=O)N[C@H]2Cc3cc(O)cc(c3)-c3ccc4c(c3)c(c(-c3cncc(C#N)c3)n4CC)CC(C)(C)COC(=O)[C@@H]3CCCN(N3)C2=O)C(C)C)C1] |
| A63 | 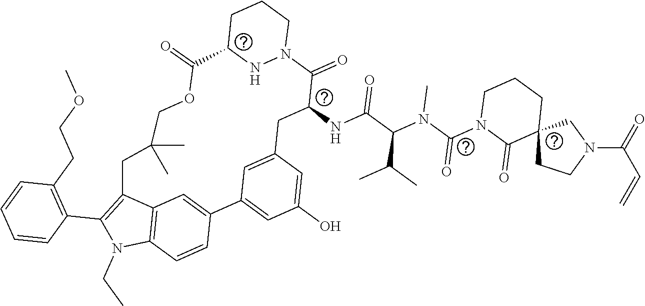 [SMILES: C=CC(=O)N1CC[C@@]2(CCCN(C(=O)N(C)[C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| A64 | 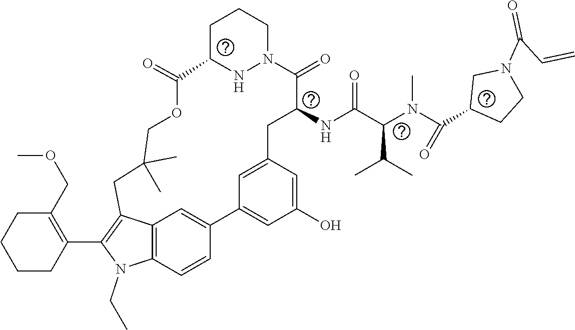 [SMILES: C=CC(=O)N1CC[C@H](C(=O)N(C)[C@H](C(=O)N[C@H]2Cc3cc(O)cc(c3)-c3ccc4c(c3)c(c(C3=C(COC)CCCC3)n4CC)CC(C)(C)COC(=O)[C@@H]3CCCN(N3)C2=O)C(C)C)C1] |
| A65 | 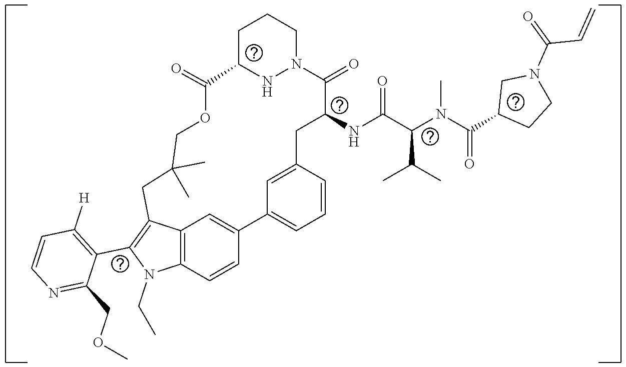 |
| A66 | 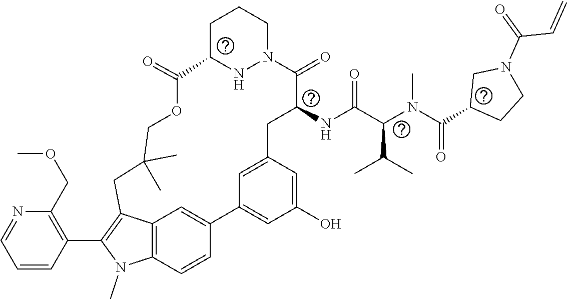 [SMILES: C=CC(=O)N1CC[C@H](C(=O)N(C)[C@H](C(=O)N[C@H]2Cc3cc(O)cc(c3)-c3ccc4c(c3)c(c(-c3cccnc3COC)n4C)CC(C)(C)COC(=O)[C@@H]3CCCN(N3)C2=O)C(C)C)C1] |
| A67 | 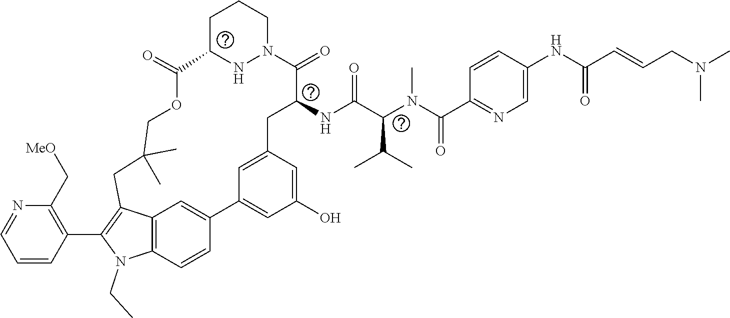 [SMILES: CCn1c(-c2cccnc2COC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| A68 | 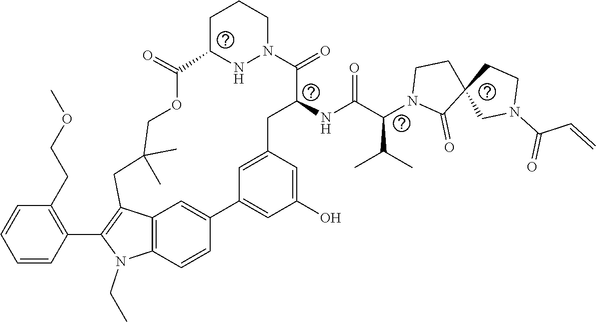 [SMILES: C=CC(=O)N1CC[C@@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| A69 | 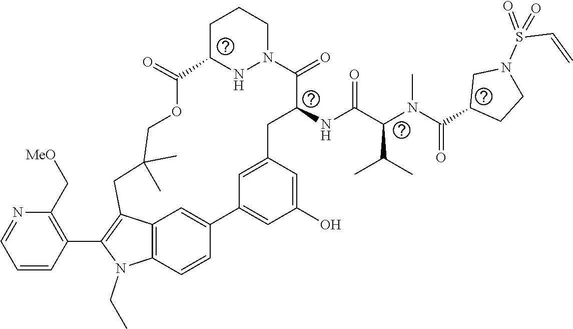 [SMILES: C=CS(=O)(=O)N1CC[C@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A70 | 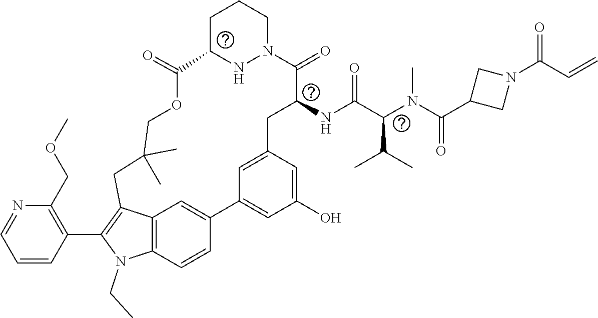 [SMILES: C=CC(=O)N1CC(C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A71 | 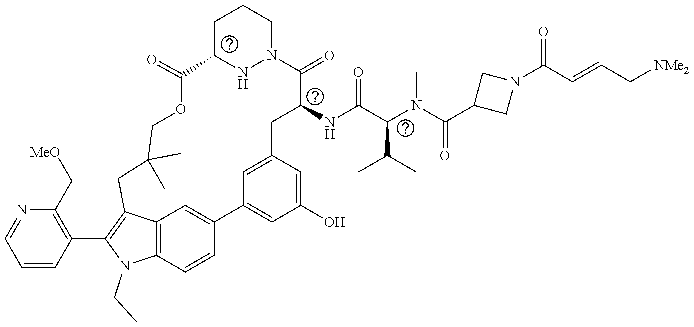 [SMILES: CCn1c(-c2cccnc2COC)c2c3cc(ccc31)-c1cc(O)cc(c1)C[C@H](NC(=O)[C@H](C(C)C)N(C)C(=O)C1CN(C(=O)/C=C/CN(C)C)C1)C(=O)N1CCC[C@H](N1)C(=O)OCC(C)(C)C2] |
| A72 | 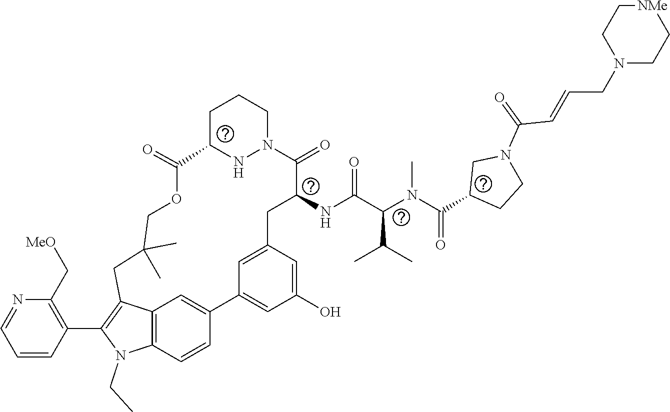 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)/C=C/CN3CCN(C)CC3)C1)C(=O)N1CCC[C@H](N1)C(=O)OCC(C)(C)C2] |
| A73 | 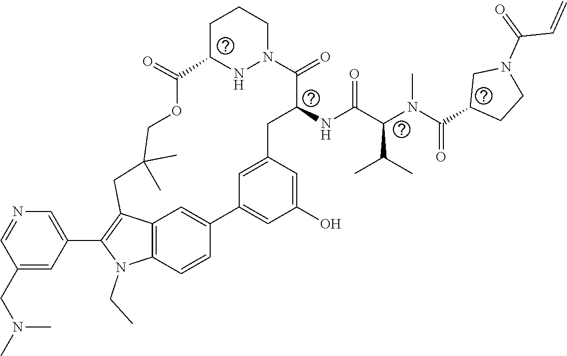 [SMILES: C=CC(=O)N1CC[C@H](C(=O)N(C)[C@H](C(=O)N[C@H]2Cc3cc(O)cc(c3)-c3ccc4c(c3)c(c(-c3cncc(CN(C)C)c3)n4CC)CC(C)(C)COC(=O)[C@@H]3CCCN(N3)C2=O)C(C)C)C1] |
| A74 | 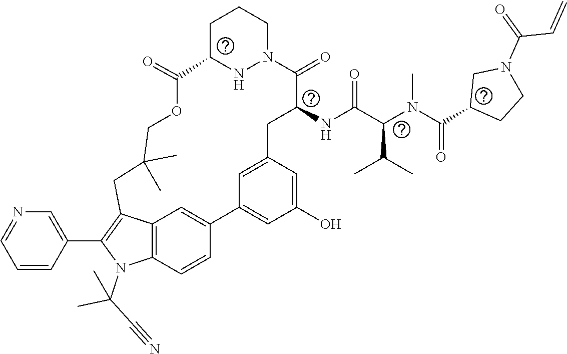 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C(C)(C)C#N)CC(C)(C)COC(=O)[C@@H]3CCCN(N3)C2=O)C(C)C)C1] |
| A75 | 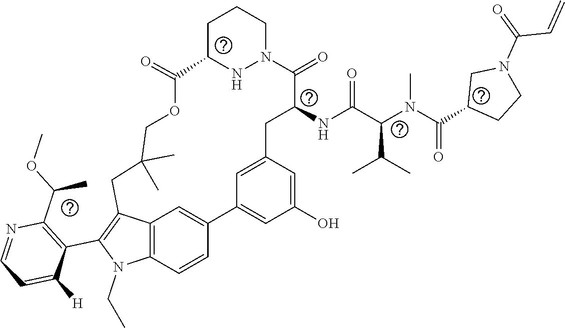 |
| A76 | 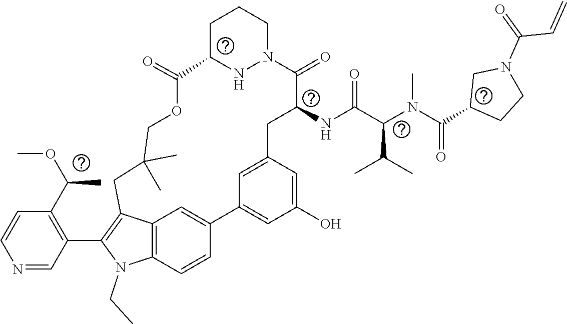 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@H](C)OC)n4CC)CC(C)(C)COC(=O)[C@@H]3CCCN(N3)C2=O)C(C)C)C1] |
| A77 | 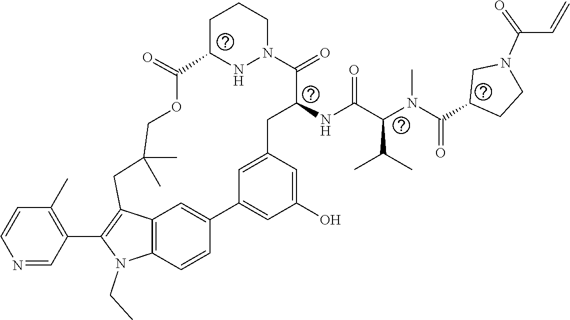 [SMILES: C=CC(=O)N1CC[C@H](C(=O)N(C)[C@H](C(=O)N[C@H]2Cc3cc(O)cc(c3)-c3ccc4c(c3)c(c(-c3cnccc3C)n4CC)CC(C)(C)COC(=O)[C@@H]3CCCN(N3)C2=O)C(C)C)C1] |
| A78 | 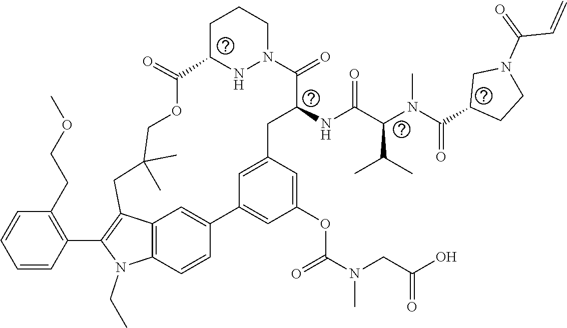 [SMILES: C=CC(=O)N1CC[C@H](C(=O)N(C)[C@H](C(=O)N[C@H]2Cc3cc(OC(=O)N(C)CC(=O)O)cc(c3)-c3ccc4c(c3)c(c(-c3ccccc3CCOC)n4CC)CC(C)(C)COC(=O)[C@@H]3CCCN(N3)C2=O)C(C)C)C1] |
| A79 | 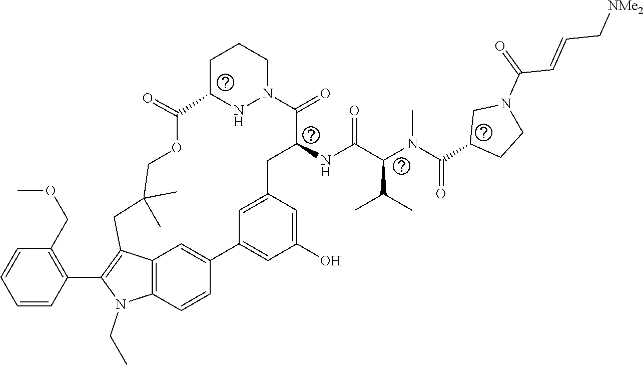 [SMILES: CCn1c(-c2ccccc2COC)c2c3cc(ccc31)-c1cc(O)cc(c1)C[C@H](NC(=O)[C@H](C(C)C)N(C)C(=O)[C@H]1CCN(C(=O)/C=C/CN(C)C)C1)C(=O)N1CCC[C@H](N1)C(=O)OCC(C)(C)C2] |
| A80 | 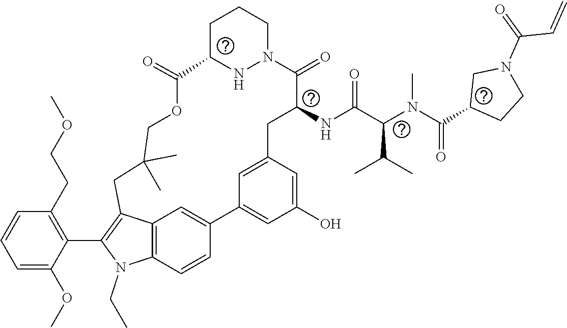 [SMILES: C=CC(=O)N1CC[C@H](C(=O)N(C)[C@H](C(=O)N[C@H]2Cc3cc(O)cc(c3)-c3ccc4c(c3)c(c(-c3c(CCOC)cccc3OC)n4CC)CC(C)(C)COC(=O)[C@@H]3CCCN(N3)C2=O)C(C)C)C1] |
| A81 | 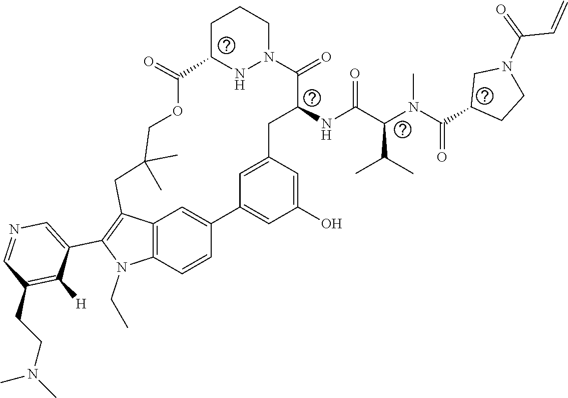 |
| A82 | 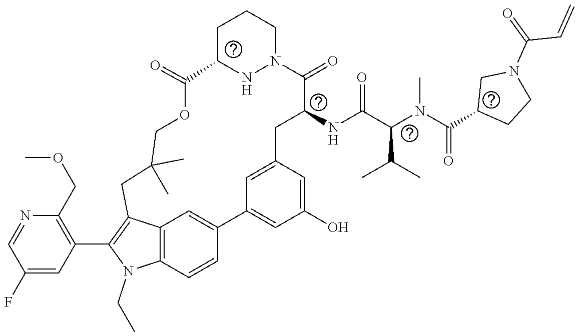 [SMILES: C=CC(=O)N1CC[C@H](C(=O)N(C)[C@H](C(=O)N[C@H]2Cc3cc(O)cc(c3)-c3ccc4c(c3)c(c(-c3cc(F)cnc3COC)n4CC)CC(C)(C)COC(=O)[C@@H]3CCCN(N3)C2=O)C(C)C)C1] |
| A83 | 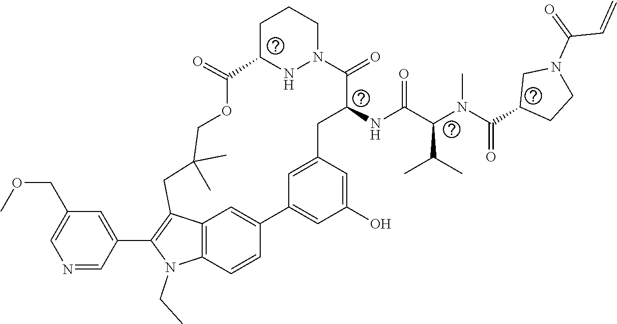 [SMILES: C=CC(=O)N1CC[C@H](C(=O)N(C)[C@H](C(=O)N[C@H]2Cc3cc(O)cc(c3)-c3ccc4c(c3)c(c(-c3cncc(COC)c3)n4CC)CC(C)(C)COC(=O)[C@@H]3CCCN(N3)C2=O)C(C)C)C1] |
| A84 | 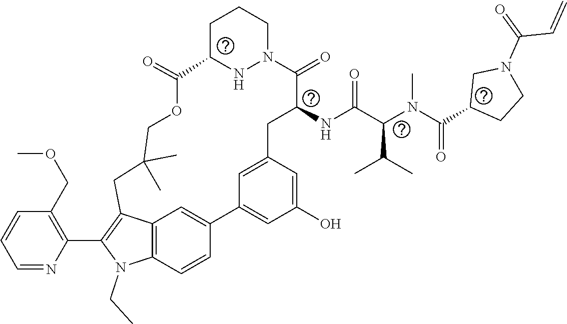 [SMILES: C=CC(=O)N1CC[C@H](C(=O)N(C)[C@H](C(=O)N[C@H]2Cc3cc(O)cc(c3)-c3ccc4c(c3)c(c(-c3ncccc3COC)n4CC)CC(C)(C)COC(=O)[C@@H]3CCCN(N3)C2=O)C(C)C)C1] |
| A85 | 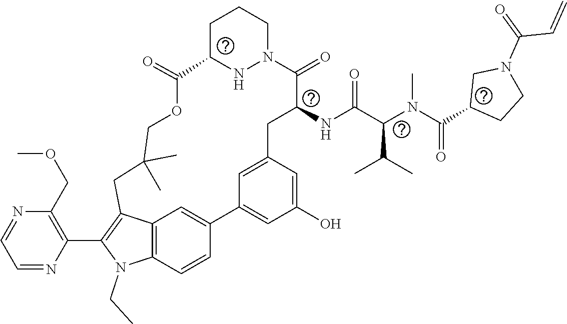 [SMILES: C=CC(=O)N1CC[C@H](C(=O)N(C)[C@H](C(=O)N[C@H]2Cc3cc(O)cc(c3)-c3ccc4c(c3)c(c(-c3nccnc3COC)n4CC)CC(C)(C)COC(=O)[C@@H]3CCCN(N3)C2=O)C(C)C)C1] |
| A86 | 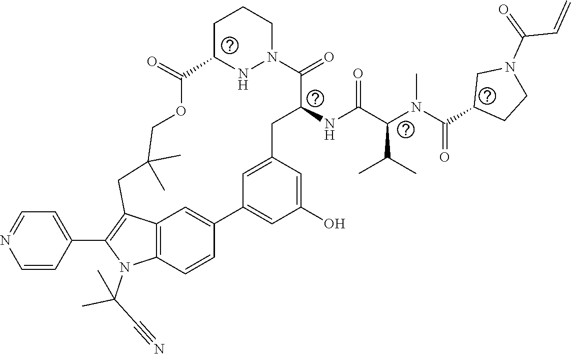 [SMILES: C=CC(=O)N1CC[C@H](C(=O)N(C)[C@H](C(=O)N[C@H]2Cc3cc(O)cc(c3)-c3ccc4c(c3)c(c(-c3ccncc3)n4C(C)(C)C#N)CC(C)(C)COC(=O)[C@@H]3CCCN(N3)C2=O)C(C)C)C1] |
| A87 | 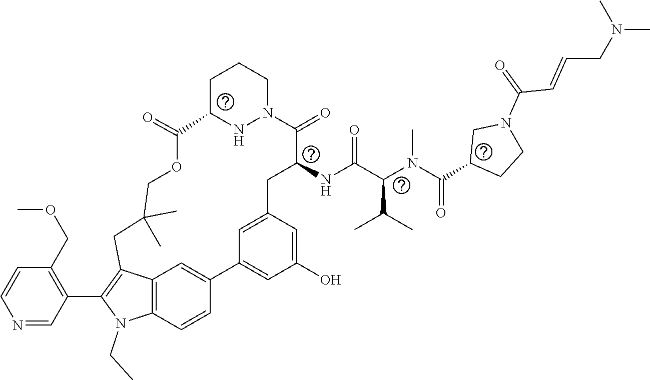 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)/C=C/CN(C)C)C1)C(=O)N1CCC[C@H](N1)C(=O)OCC(C)(C)C2] |
| A88 | 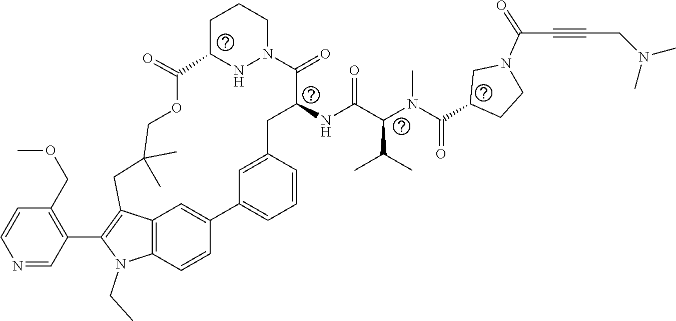 [SMILES: CCn1c(-c2cnccc2COC)c2c3cc(ccc31)-c1cccc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A89 | 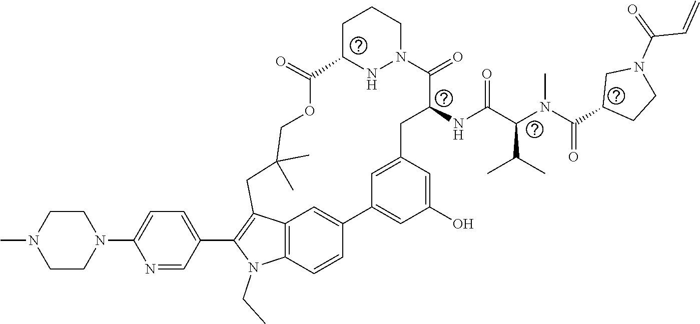 [SMILES: C=CC(=O)N1CC[C@H](C(=O)N(C)[C@H](C(=O)N[C@H]2Cc3cc(O)cc(c3)-c3ccc4c(c3)c(c(-c3ccc(N5CCN(C)CC5)nc3)n4CC)CC(C)(C)COC(=O)[C@@H]3CCCN(N3)C2=O)C(C)C)C1] |
| A90 | 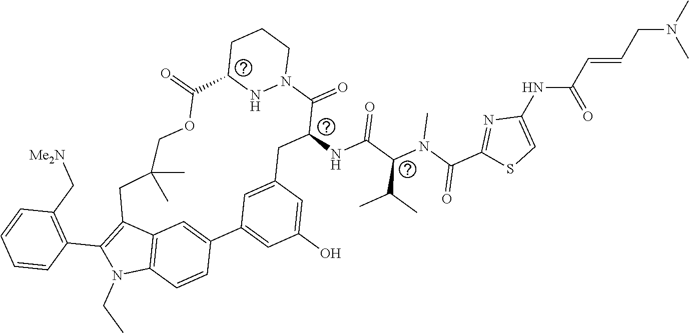 [SMILES: CCn1c(-c2ccccc2CN(C)C)c2c3cc(ccc31)-c1cc(O)cc(c1)C[C@H](NC(=O)[C@H](C(C)C)N(C)C(=O)c1nc(NC(=O)/C=C/CN(C)C)cs1)C(=O)N1CCC[C@H](N1)C(=O)OCC(C)(C)C2] |
| A91 | 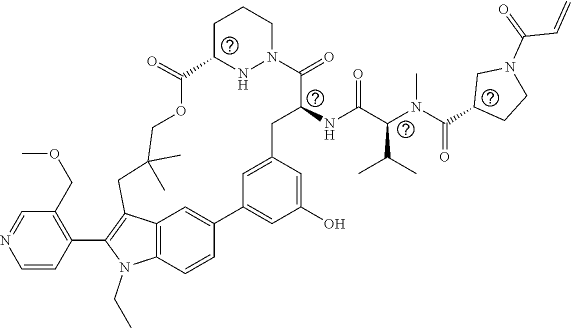 [SMILES: C=CC(=O)N1CC[C@H](C(=O)N(C)[C@H](C(=O)N[C@H]2Cc3cc(O)cc(c3)-c3ccc4c(c3)c(c(-c3ccncc3COC)n4CC)CC(C)(C)COC(=O)[C@@H]3CCCN(N3)C2=O)C(C)C)C1] |
| A92 | 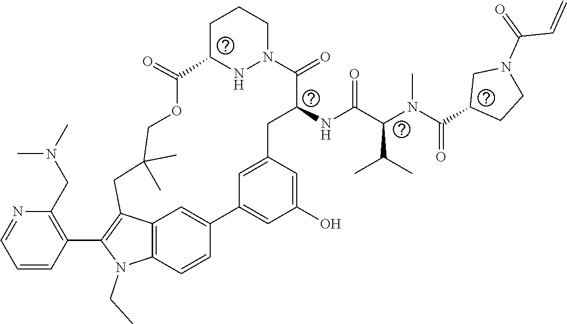 [SMILES: C=CC(=O)N1CC[C@H](C(=O)N(C)[C@H](C(=O)N[C@H]2Cc3cc(O)cc(c3)-c3ccc4c(c3)c(c(-c3cccnc3CN(C)C)n4CC)CC(C)(C)COC(=O)[C@@H]3CCCN(N3)C2=O)C(C)C)C1] |
| A93 | 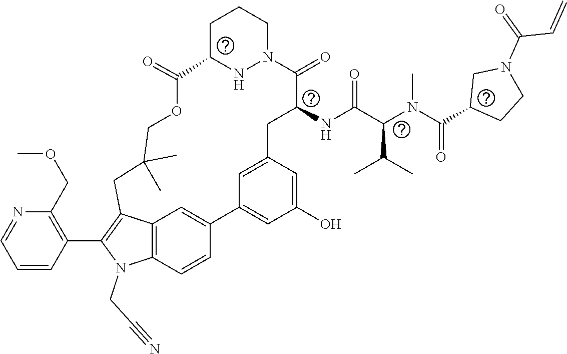 [SMILES: C=CC(=O)N1CC[C@H](C(=O)N(C)[C@H](C(=O)N[C@H]2Cc3cc(O)cc(c3)-c3ccc4c(c3)c(c(-c3cccnc3COC)n4CC#N)CC(C)(C)COC(=O)[C@@H]3CCCN(N3)C2=O)C(C)C)C1] |
| A94 | 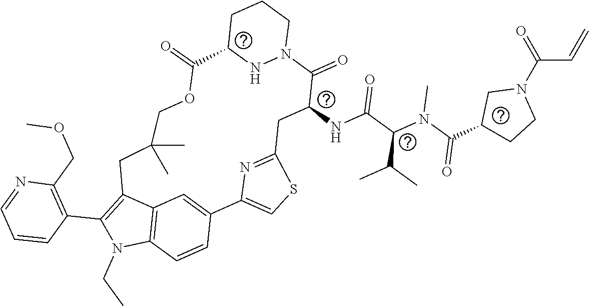 [SMILES: C=CC(=O)N1CC[C@H](C(=O)N(C)[C@H](C(=O)N[C@H]2Cc3nc(cs3)-c3ccc4c(c3)c(c(-c3cccnc3COC)n4CC)CC(C)(C)COC(=O)[C@@H]3CCCN(N3)C2=O)C(C)C)C1] |
| A95 | 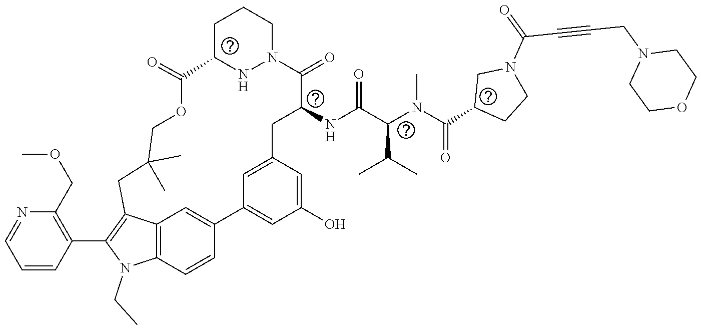 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A96 | 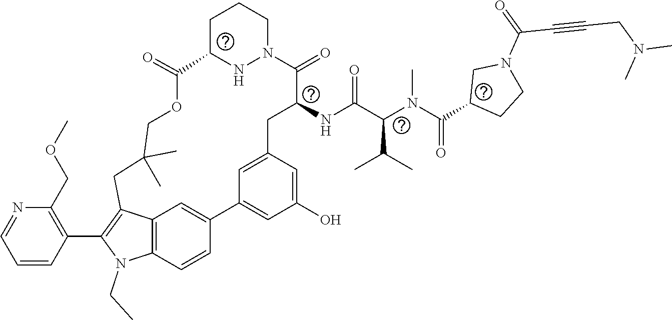 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A97 | 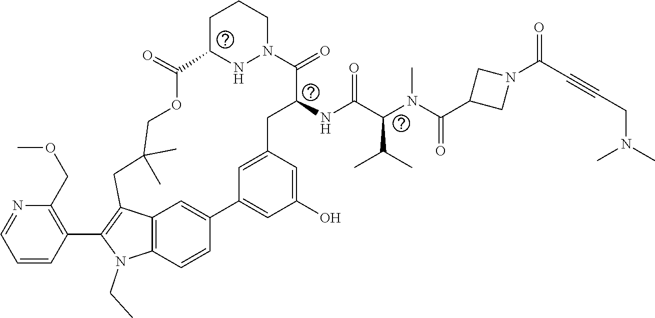 [SMILES: CCn1c(-c2cccnc2COC)c2c3cc(ccc31)-c1cc(O)cc(c1)C[C@H](NC(=O)[C@H](C(C)C)N(C)C(=O)C1CN(C(=O)C#CCN(C)C)C1)C(=O)N1CCC[C@H](N1)C(=O)OCC(C)(C)C2] |
| A98 | 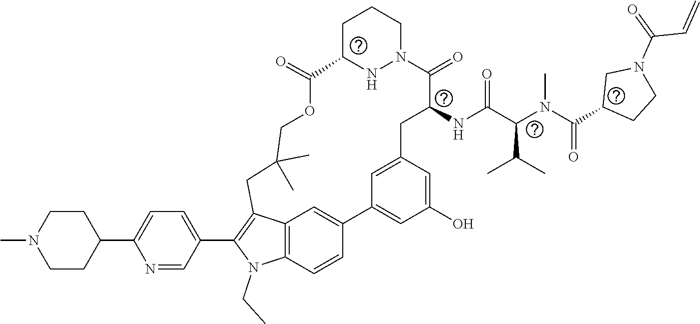 [SMILES: C=CC(=O)N1CC[C@H](C(=O)N(C)[C@H](C(=O)N[C@H]2Cc3cc(O)cc(c3)-c3ccc4c(c3)c(c(-c3ccc(C5CCN(C)CC5)nc3)n4CC)CC(C)(C)COC(=O)[C@@H]3CCCN(N3)C2=O)C(C)C)C1] |
| A99 | 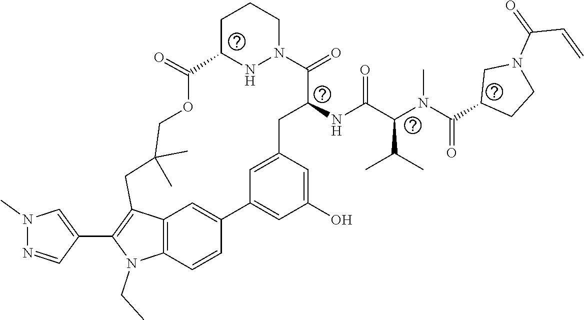 [SMILES: C=CC(=O)N1CC[C@H](C(=O)N(C)[C@H](C(=O)N[C@H]2Cc3cc(O)cc(c3)-c3ccc4c(c3)c(c(-c3cnn(C)c3)n4CC)CC(C)(C)COC(=O)[C@@H]3CCCN(N3)C2=O)C(C)C)C1] |
| A100 | 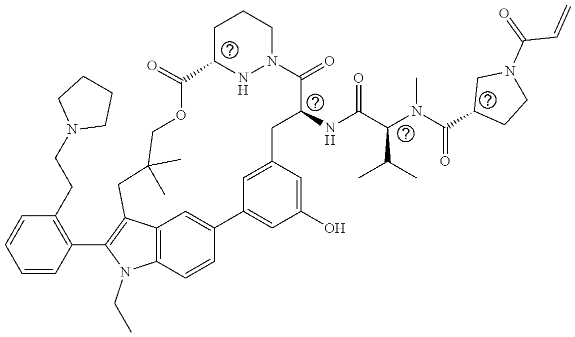 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCN3CCCC3)n4CC)CC(C)(C)COC(=O)[C@@H]3CCCN(N3)C2=O)C(C)C)C1] |
| A101 | 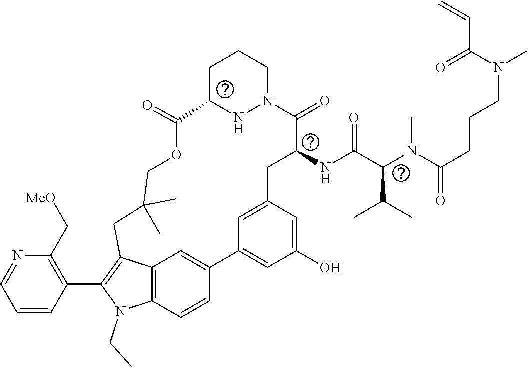 [SMILES: C=CC(=O)N(C)CCCC(=O)N(C)[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| A102 | 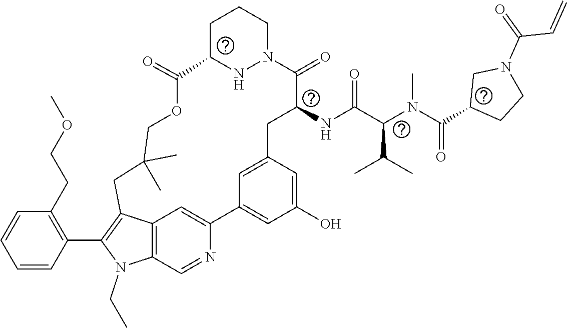 [SMILES: C=CC(=O)N1CC[C@H](C(=O)N(C)[C@H](C(=O)N[C@H]2Cc3cc(O)cc(c3)-c3cc4c(c(-c5ccccc5CCOC)n(CC)c4cn3)CC(C)(C)COC(=O)[C@@H]3CCCN(N3)C2=O)C(C)C)C1] |
| A103 | 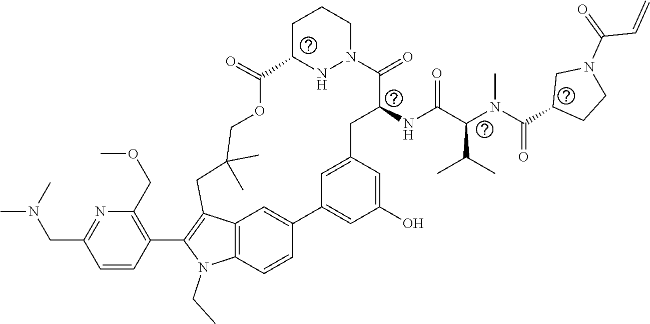 [SMILES: C=CC(=O)N1CC[C@H](C(=O)N(C)[C@H](C(=O)N[C@H]2Cc3cc(O)cc(c3)-c3ccc4c(c3)c(c(-c3ccc(CN(C)C)nc3COC)n4CC)CC(C)(C)COC(=O)[C@@H]3CCCN(N3)C2=O)C(C)C)C1] |
| A104 | 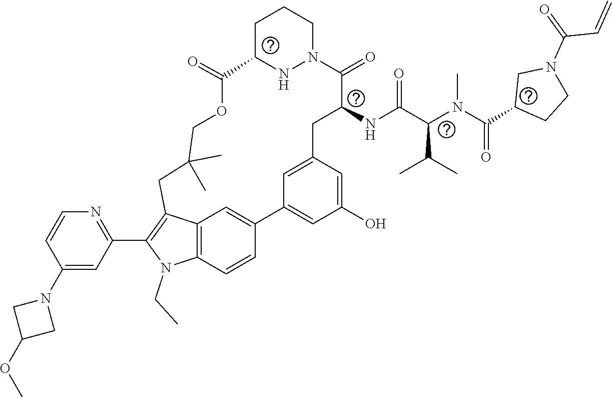 [SMILES: C=CC(=O)N1CC[C@H](C(=O)N(C)[C@H](C(=O)N[C@H]2Cc3cc(O)cc(c3)-c3ccc4c(c3)c(c(-c3cc(N5CC(OC)C5)ccn3)n4CC)CC(C)(C)COC(=O)[C@@H]3CCCN(N3)C2=O)C(C)C)C1] |
| A105 | 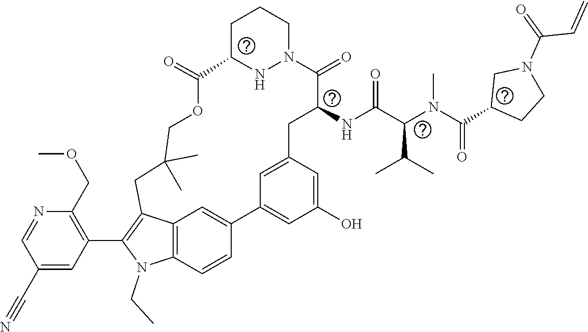 [SMILES: C=CC(=O)N1CC[C@H](C(=O)N(C)[C@H](C(=O)N[C@H]2Cc3cc(O)cc(c3)-c3ccc4c(c3)c(c(-c3cc(C#N)cnc3COC)n4CC)CC(C)(C)COC(=O)[C@@H]3CCCN(N3)C2=O)C(C)C)C1] |
| A106 | 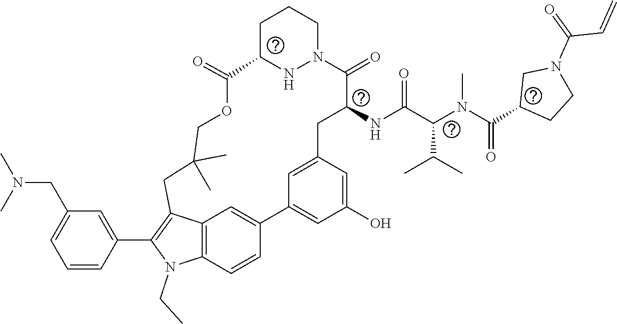 [SMILES: C=CC(=O)N1CC[C@H](C(=O)N(C)[C@@H](C(=O)N[C@H]2Cc3cc(O)cc(c3)-c3ccc4c(c3)c(c(-c3cccc(CN(C)C)c3)n4CC)CC(C)(C)COC(=O)[C@@H]3CCCN(N3)C2=O)C(C)C)C1] |
| A107 | 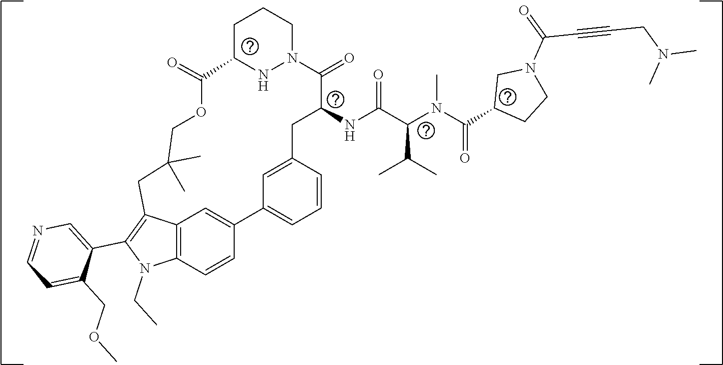 |
| A108 | 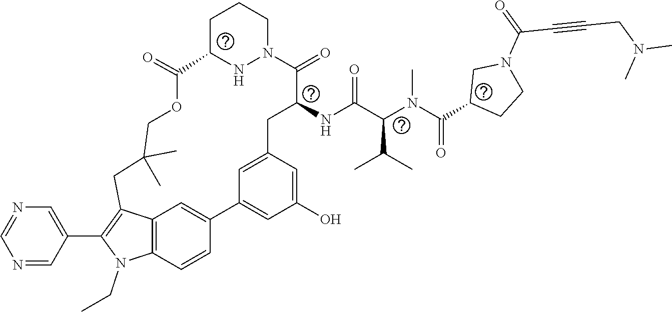 [SMILES: CCn1c(-c2cncnc2)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A109 | 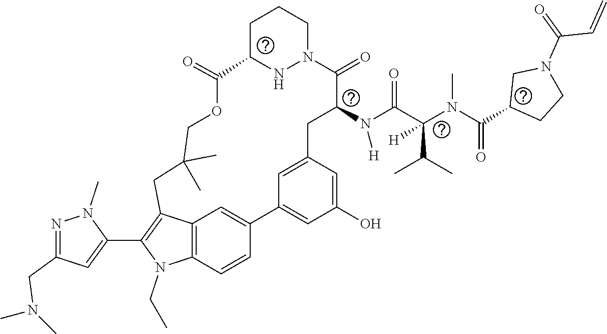 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)nn3C)n4CC)CC(C)(C)COC(=O)[C@@H]3CCCN(N3)C2=O)C(C)C)C1] |
| A110 | 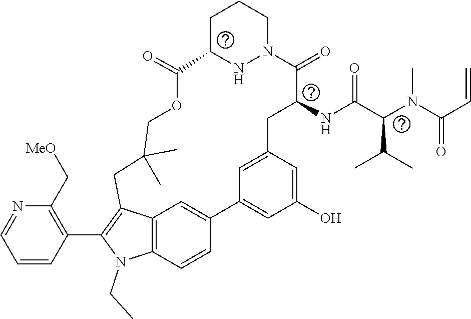 [SMILES: C=CC(=O)N(C)[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| A111 | 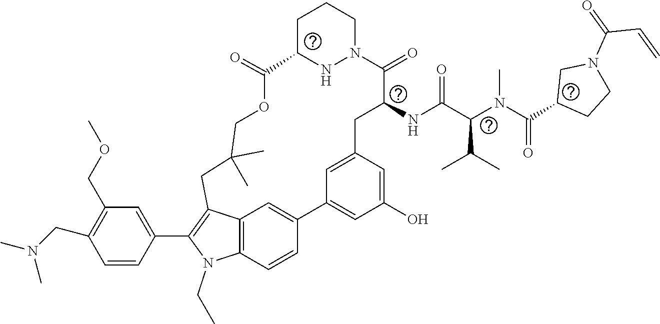 [SMILES: C=CC(=O)N1CC[C@H](C(=O)N(C)[C@H](C(=O)N[C@H]2Cc3cc(O)cc(c3)-c3ccc4c(c3)c(c(-c3ccc(CN(C)C)c(COC)c3)n4CC)CC(C)(C)COC(=O)[C@@H]3CCCN(N3)C2=O)C(C)C)C1] |
| A112 | 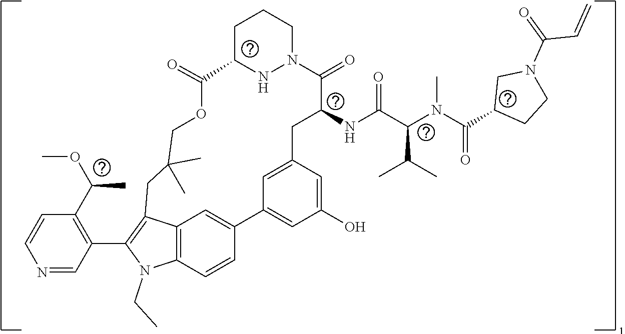 |
| A113 | 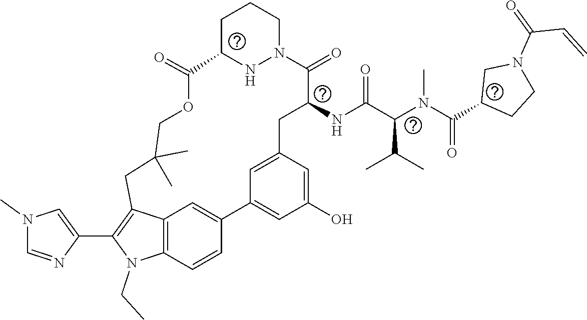 [SMILES: C=CC(=O)N1CC[C@H](C(=O)N(C)[C@H](C(=O)N[C@H]2Cc3cc(O)cc(c3)-c3ccc4c(c3)c(c(-c3cn(C)cn3)n4CC)CC(C)(C)COC(=O)[C@@H]3CCCN(N3)C2=O)C(C)C)C1] |
| A114 | 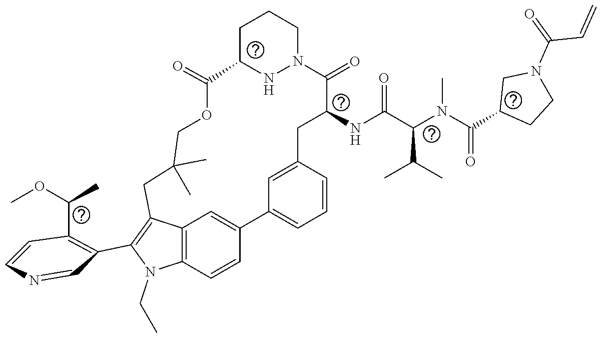 |
| A115 | 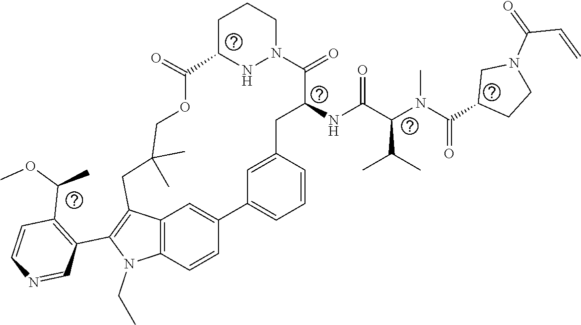 |
| A116 | 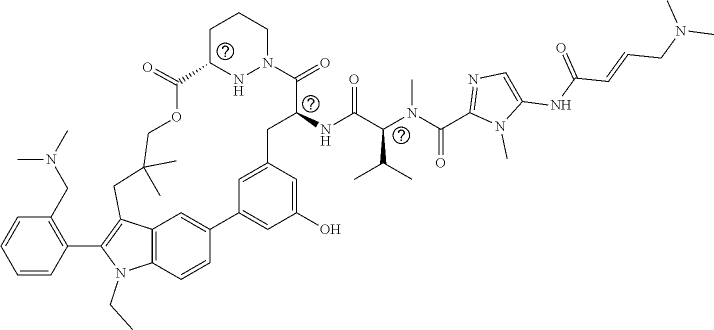 [SMILES: CCn1c(-c2ccccc2CN(C)C)c2c3cc(ccc31)-c1cc(O)cc(c1)C[C@H](NC(=O)[C@H](C(C)C)N(C)C(=O)c1ncc(NC(=O)/C=C/CN(C)C)n1C)C(=O)N1CCC[C@H](N1)C(=O)OCC(C)(C)C2] |
| A117 | 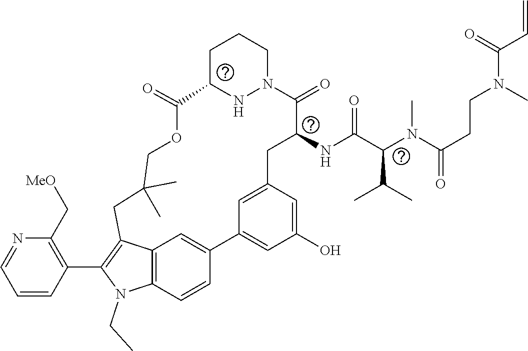 [SMILES: C=CC(=O)N(C)CCC(=O)N(C)[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| A118 | 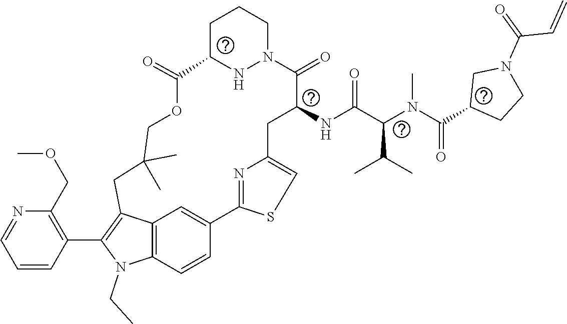 [SMILES: C=CC(=O)N1CC[C@H](C(=O)N(C)[C@H](C(=O)N[C@H]2Cc3csc(n3)-c3ccc4c(c3)c(c(-c3cccnc3COC)n4CC)CC(C)(C)COC(=O)[C@@H]3CCCN(N3)C2=O)C(C)C)C1] |
| A119 | 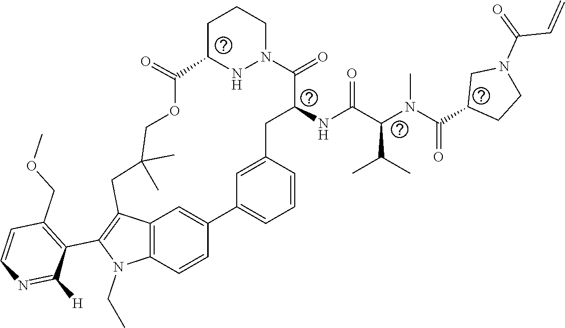 |
| A120 | 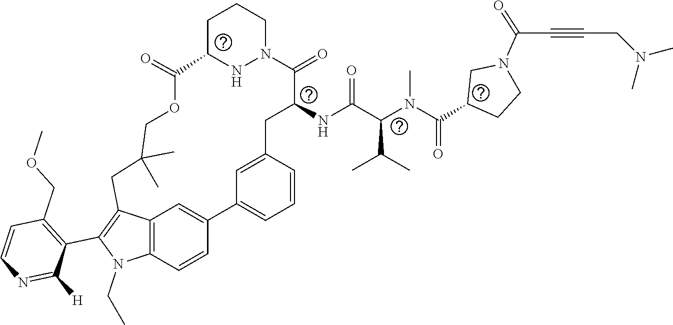 |
| A121 | 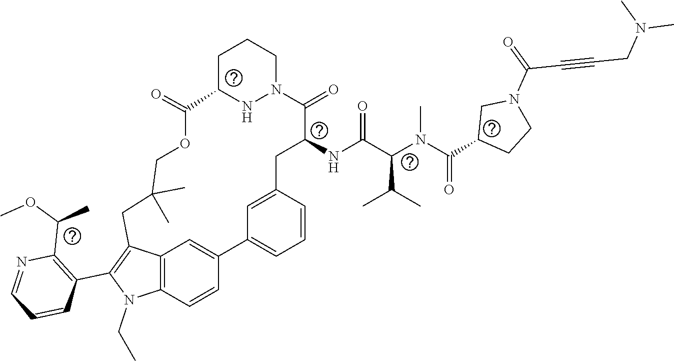 |
| A122 | 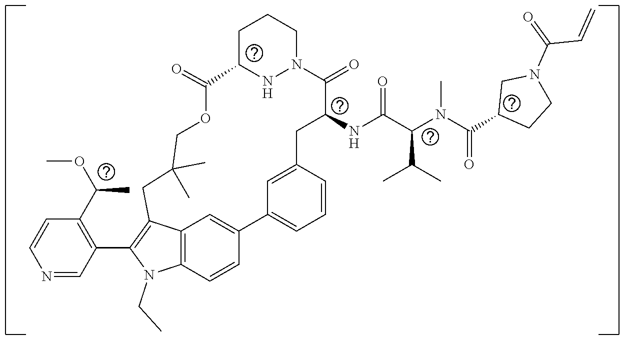 |
| A123 | 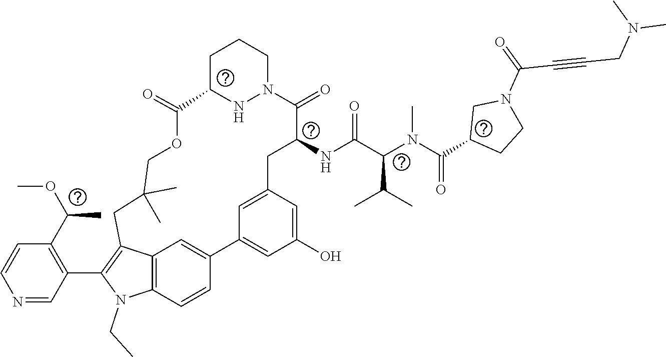 [SMILES: CCn1c(-c2cnccc2[C@H](C)OC)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A124 | 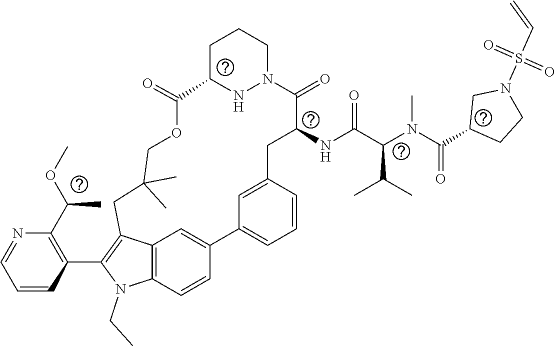 |
| A125 | 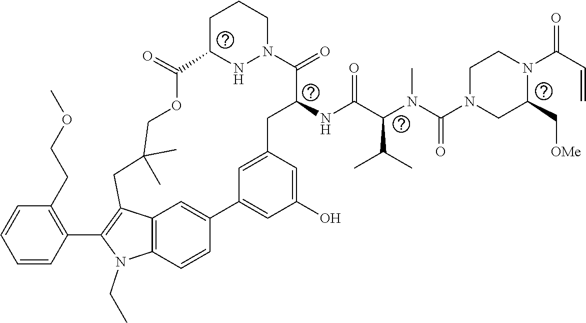 [SMILES: C=CC(=O)N1CCN(C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C[C@@H]1COC] |
| A126 | 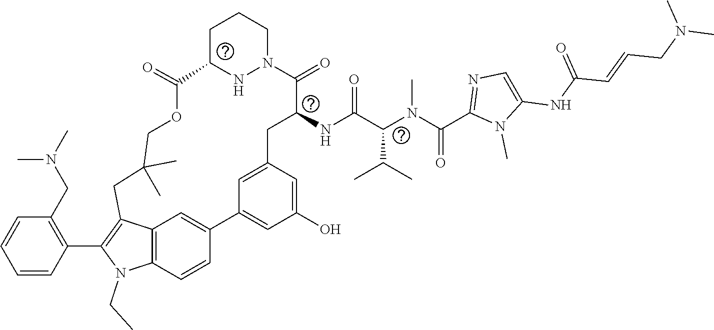 [SMILES: CCn1c(-c2ccccc2CN(C)C)c2c3cc(ccc31)-c1cc(O)cc(c1)C[C@H](NC(=O)[C@@H](C(C)C)N(C)C(=O)c1ncc(NC(=O)/C=C/CN(C)C)n1C)C(=O)N1CCC[C@H](N1)C(=O)OCC(C)(C)C2] |
| A127 | 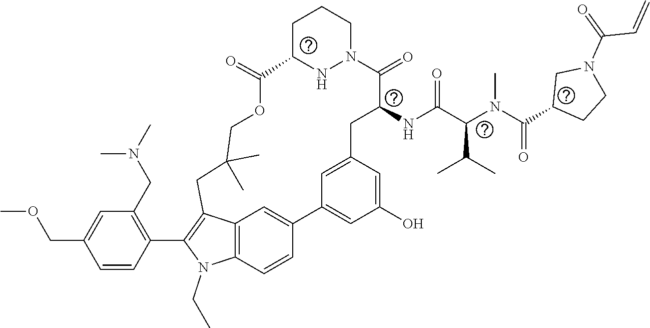 [SMILES: C=CC(=O)N1CC[C@H](C(=O)N(C)[C@H](C(=O)N[C@H]2Cc3cc(O)cc(c3)-c3ccc4c(c3)c(c(-c3ccc(COC)cc3CN(C)C)n4CC)CC(C)(C)COC(=O)[C@@H]3CCCN(N3)C2=O)C(C)C)C1] |
| A128 | 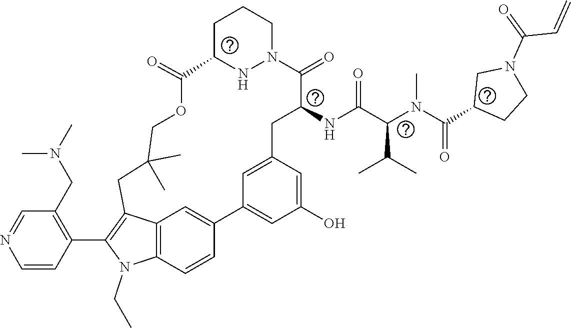 [SMILES: C=CC(=O)N1CC[C@H](C(=O)N(C)[C@H](C(=O)N[C@H]2Cc3cc(O)cc(c3)-c3ccc4c(c3)c(c(-c3ccncc3CN(C)C)n4CC)CC(C)(C)COC(=O)[C@@H]3CCCN(N3)C2=O)C(C)C)C1] |
| A129 | 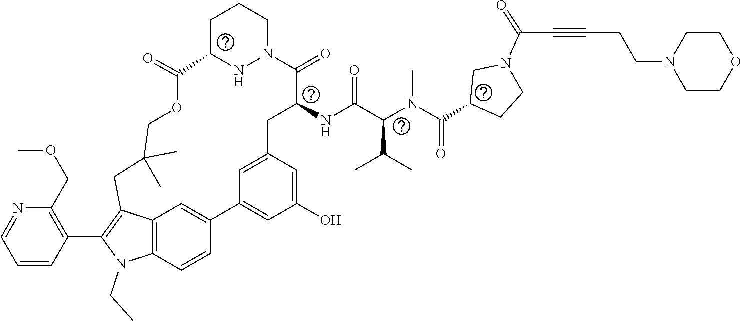 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCCN3CCOCC3)C1)C(=O)N1CCC[C@H](N1)C(=O)OCC(C)(C)C2] |
| A130 | 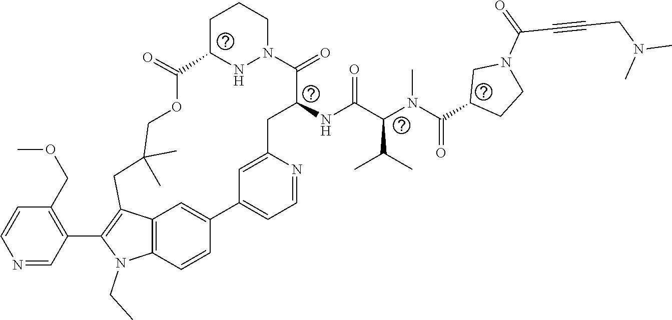 [SMILES: CCn1c(-c2cnccc2COC)c2c3cc(ccc31)-c1ccnc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A131 | 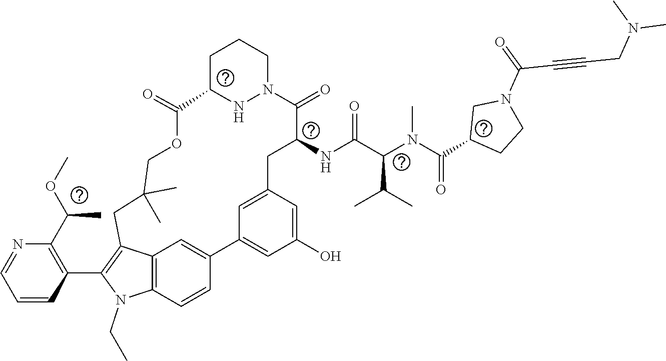 |
| A132 | 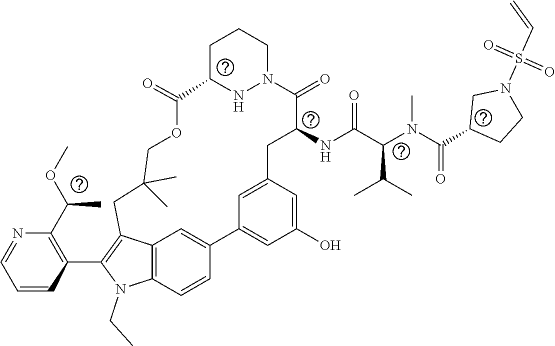 |
| A133 | 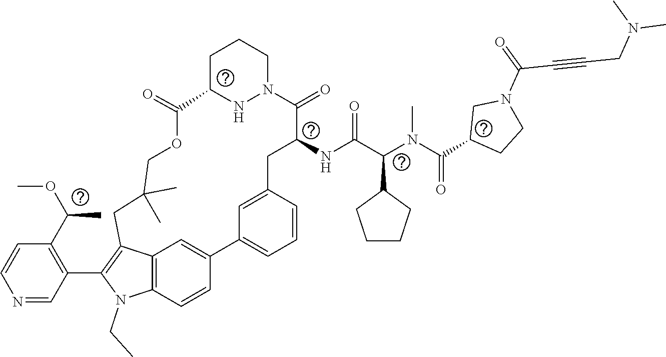 [SMILES: CCn1c(-c2cnccc2[C@H](C)OC)c2c3cc(ccc31)-c1cccc(c1)C[C@H](NC(=O)[C@H](C1CCCC1)N(C)C(=O)[C@H]1CCN(C(=O)C#CCN(C)C)C1)C(=O)N1CCC[C@H](N1)C(=O)OCC(C)(C)C2] |
| A134 | 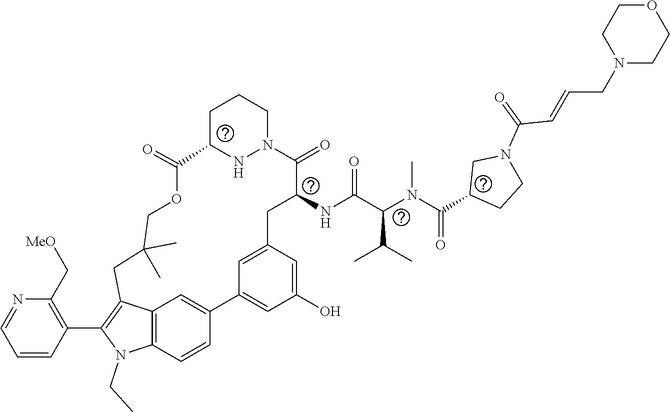 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)/C=C/CN3CCOCC3)C1)C(=O)N1CCC[C@H](N1)C(=O)OCC(C)(C)C2] |
| A135 | 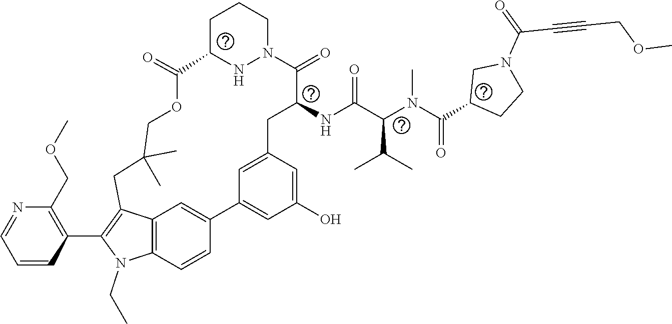 |
| A136 | 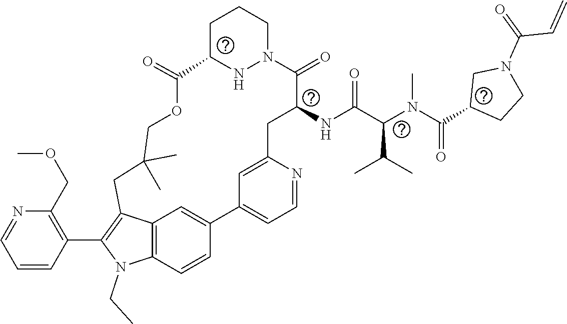 [SMILES: C=CC(=O)N1CC[C@H](C(=O)N(C)[C@H](C(=O)N[C@H]2Cc3cc(ccn3)-c3ccc4c(c3)c(c(-c3cccnc3COC)n4CC)CC(C)(C)COC(=O)[C@@H]3CCCN(N3)C2=O)C(C)C)C1] |
| A137 | 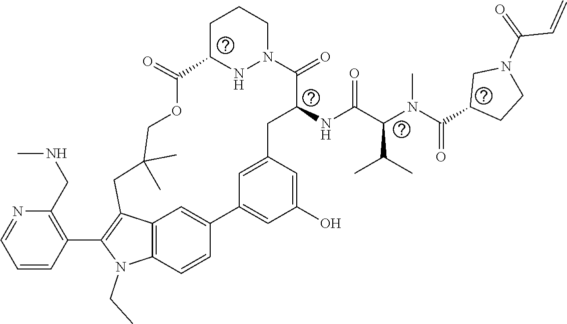 [SMILES: C=CC(=O)N1CC[C@H](C(=O)N(C)[C@H](C(=O)N[C@H]2Cc3cc(O)cc(c3)-c3ccc4c(c3)c(c(-c3cccnc3CNC)n4CC)CC(C)(C)COC(=O)[C@@H]3CCCN(N3)C2=O)C(C)C)C1] |
| A138 | 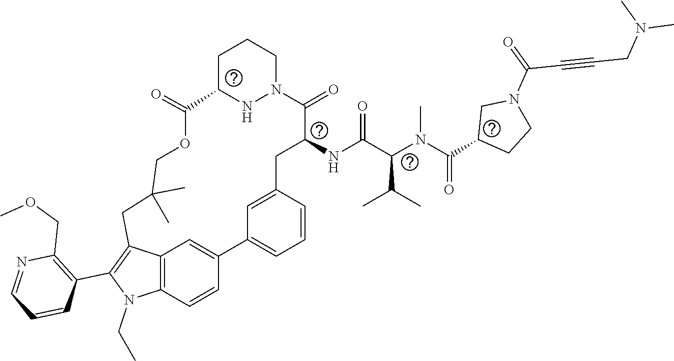 |
| A139 | 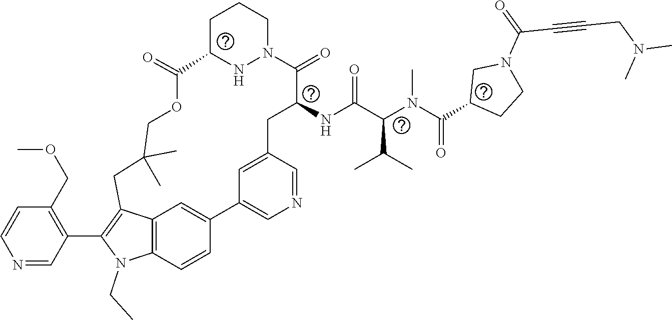 [SMILES: CCn1c(-c2cnccc2COC)c2c3cc(ccc31)-c1cncc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A140 | 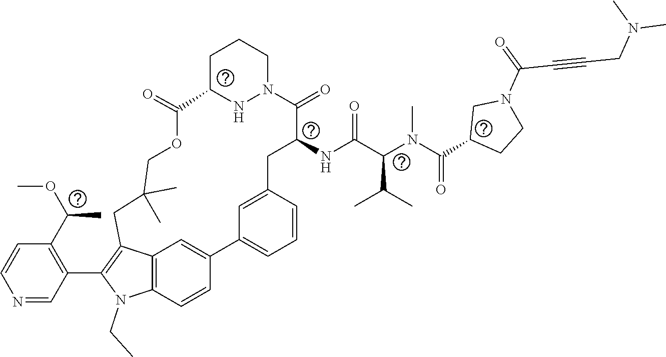 [SMILES: CCn1c(-c2cnccc2[C@H](C)OC)c2c3cc(ccc31)-c1cccc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A141 | 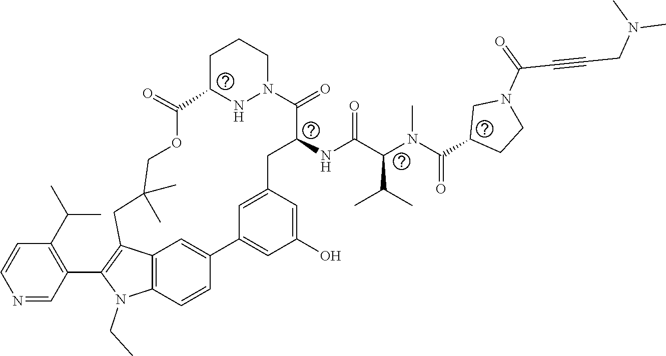 [SMILES: CCn1c(-c2cnccc2C(C)C)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A142 | 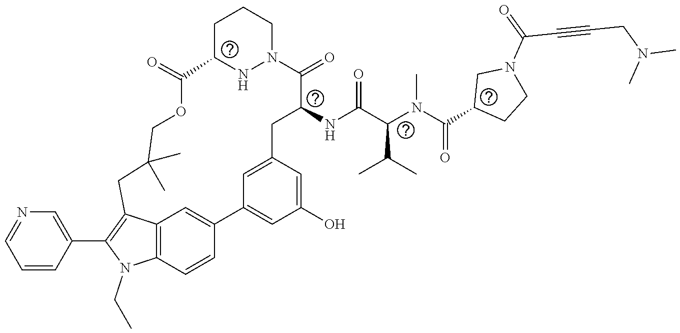 [SMILES: CCn1c(-c2cccnc2)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A143 | 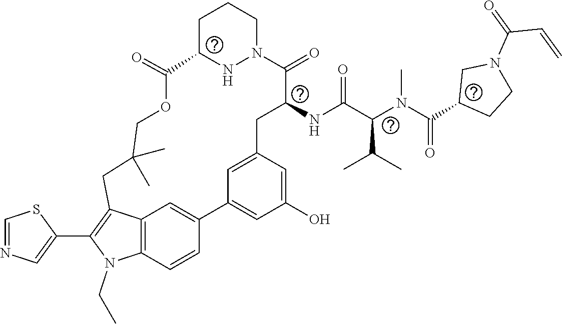 [SMILES: C=CC(=O)N1CC[C@H](C(=O)N(C)[C@H](C(=O)N[C@H]2Cc3cc(O)cc(c3)-c3ccc4c(c3)c(c(-c3cncs3)n4CC)CC(C)(C)COC(=O)[C@@H]3CCCN(N3)C2=O)C(C)C)C1] |
| A144 | 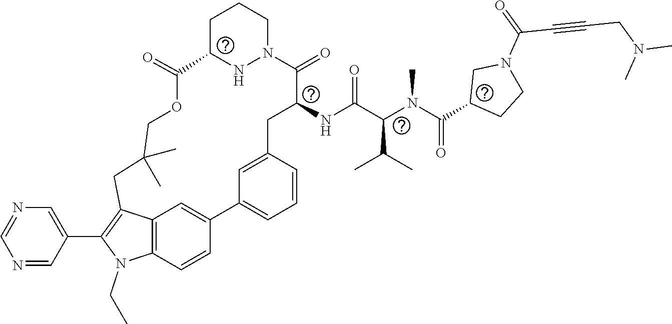 |
| A145 | 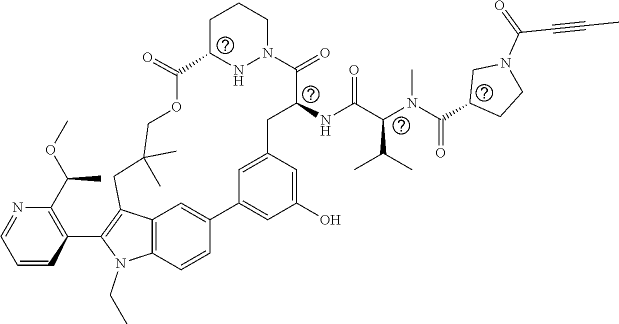 |
| A146 | 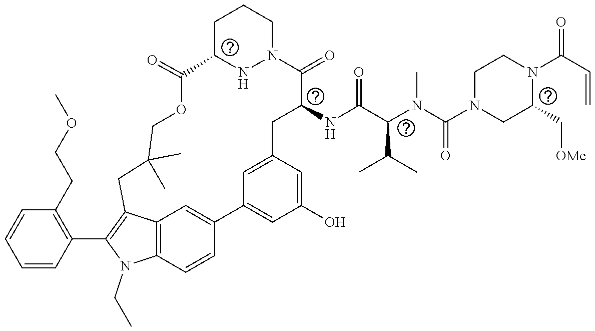 [SMILES: C=CC(=O)N1CCN(C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C[C@H]1COC] |
| A147 | 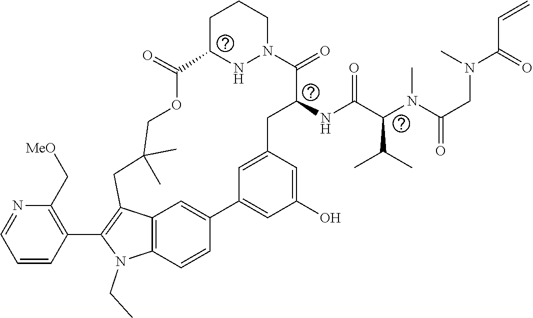 [SMILES: C=CC(=O)N(C)CC(=O)N(C)[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| A148 | 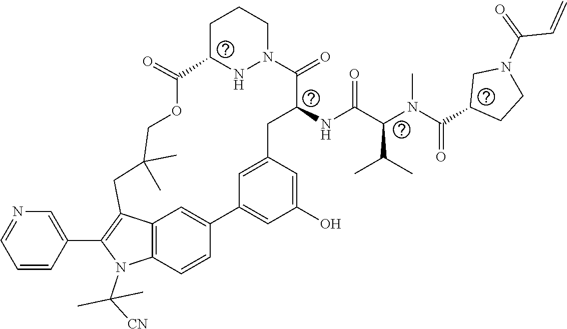 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C(C)(C)C#N)CC(C)(C)COC(=O)[C@@H]3CCCN(N3)C2=O)C(C)C)C1] |
| A149 | 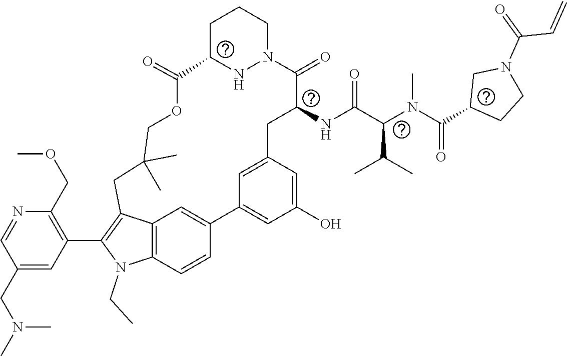 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3COC)n4CC)CC(C)(C)COC(=O)[C@@H]3CCCN(N3)C2=O)C(C)C)C1] |
| A150 | 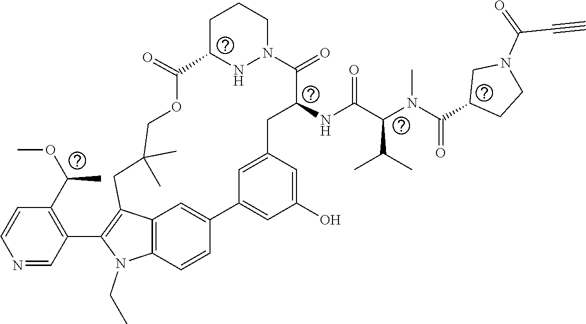 [SMILES: C#CC(=O)N1CC[C@H](C(=O)N(C)[C@H](C(=O)N[C@H]2Cc3cc(O)cc(c3)-c3ccc4c(c3)c(c(-c3cnccc3[C@H](C)OC)n4CC)CC(C)(C)COC(=O)[C@@H]3CCCN(N3)C2=O)C(C)C)C1] |
| A151 | 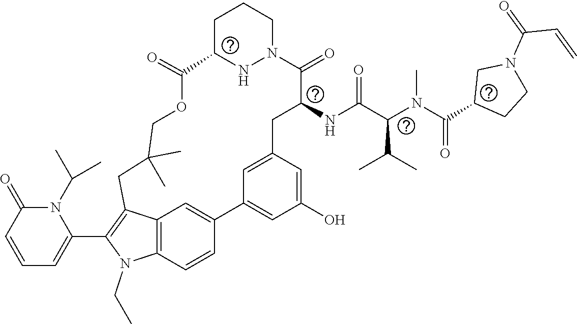 [SMILES: C=CC(=O)N1CC[C@H](C(=O)N(C)[C@H](C(=O)N[C@H]2Cc3cc(O)cc(c3)-c3ccc4c(c3)c(c(-c3cccc(=O)n3C(C)C)n4CC)CC(C)(C)COC(=O)[C@@H]3CCCN(N3)C2=O)C(C)C)C1] |
| A152 | 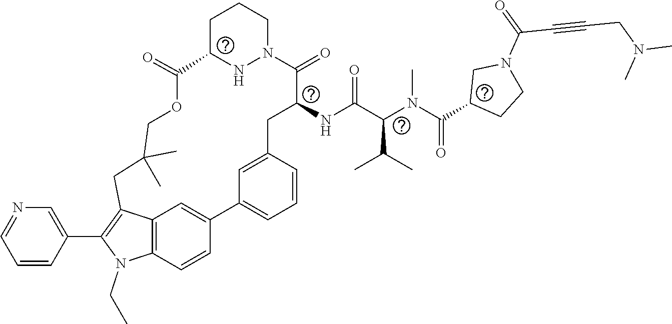 [SMILES: CCn1c(-c2cccnc2)c2c3cc(ccc31)-c1cccc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A153 | 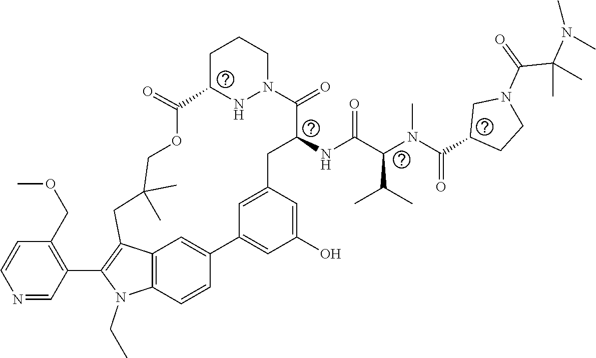 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A154 | 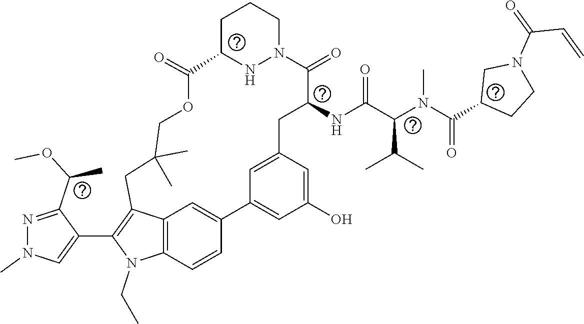 [SMILES: C=CC(=O)N1CC[C@H](C(=O)N(C)[C@H](C(=O)N[C@H]2Cc3cc(O)cc(c3)-c3ccc4c(c3)c(c(-c3cn(C)nc3[C@H](C)OC)n4CC)CC(C)(C)COC(=O)[C@@H]3CCCN(N3)C2=O)C(C)C)C1] |
| A155 | 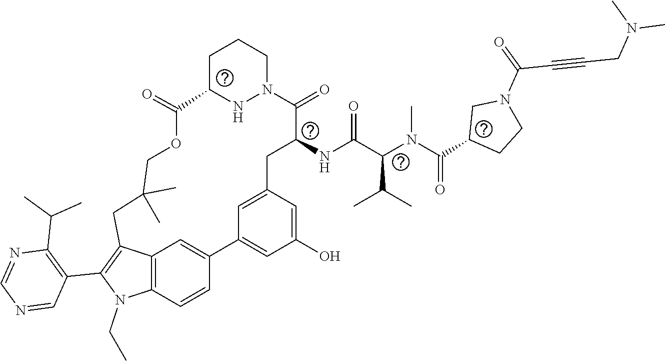 [SMILES: CCn1c(-c2cncnc2C(C)C)c2c3cc(ccc31)-c1cc(O)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A156 | 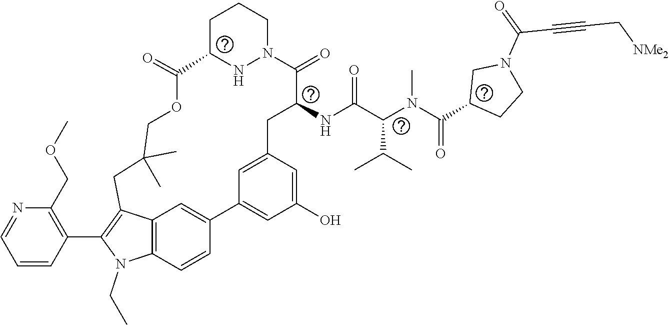 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CCN(C)C)C1)C(=O)N1CCC[C@H](N1)C(=O)OCC(C)(C)C2] |
| A157 | 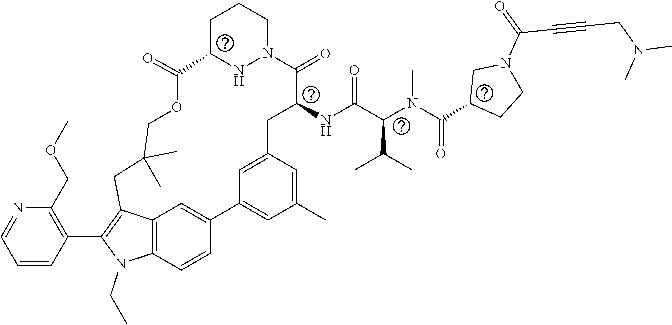 [SMILES: CCn1c(-c2cccnc2COC)c2c3cc(ccc31)-c1cc(C)cc(c1)C[C@H](NC(=O)[C@H](C(C)C)N(C)C(=O)[C@H]1CCN(C(=O)C#CCN(C)C)C1)C(=O)N1CCC[C@H](N1)C(=O)OCC(C)(C)C2] |
| A158 | 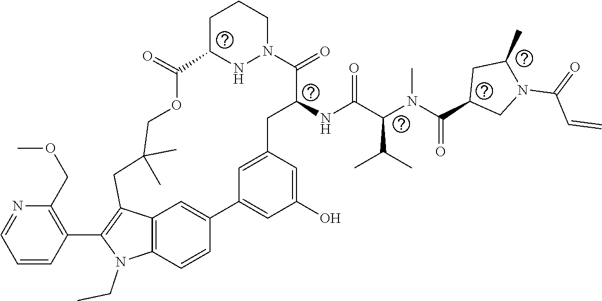 [SMILES: C=CC(=O)N1C[C@@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C[C@H]1C] |
| A159 | 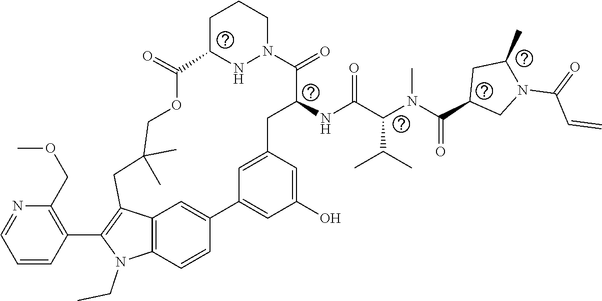 [SMILES: C=CC(=O)N1C[C@@H](C(=O)N(C)[C@@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C[C@H]1C] |
| A160 | 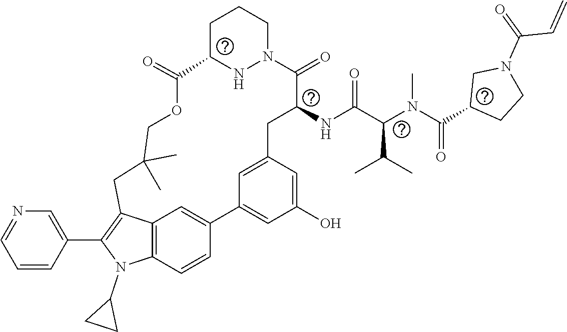 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3CC3)CC(C)(C)COC(=O)[C@@H]3CCCN(N3)C2=O)C(C)C)C1] |
| A161 | 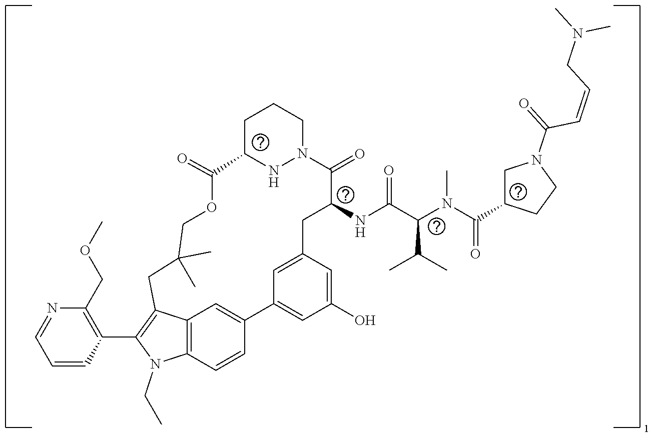 |
| A162 | 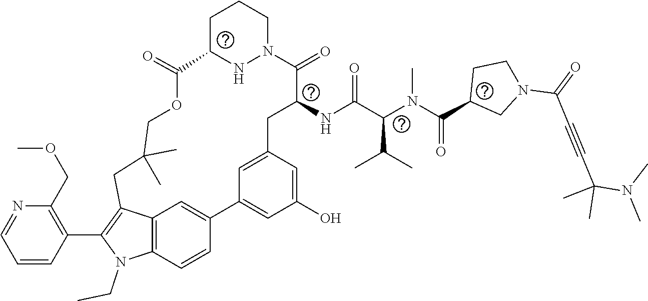 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC(C)(C)N(C)C)C1)C(=O)N1CCC[C@H](N1)C(=O)OCC(C)(C)C2] |
| A163 | 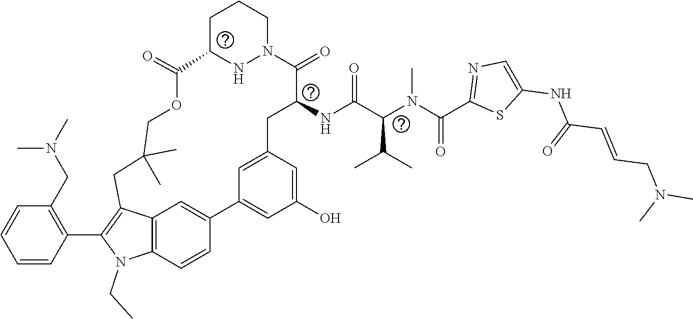 [SMILES: CCn1c(-c2ccccc2CN(C)C)c2c3cc(ccc31)-c1cc(O)cc(c1)C[C@H](NC(=O)[C@H](C(C)C)N(C)C(=O)c1ncc(NC(=O)/C=C/CN(C)C)s1)C(=O)N1CCC[C@H](N1)C(=O)OCC(C)(C)C2] |
| A164 | 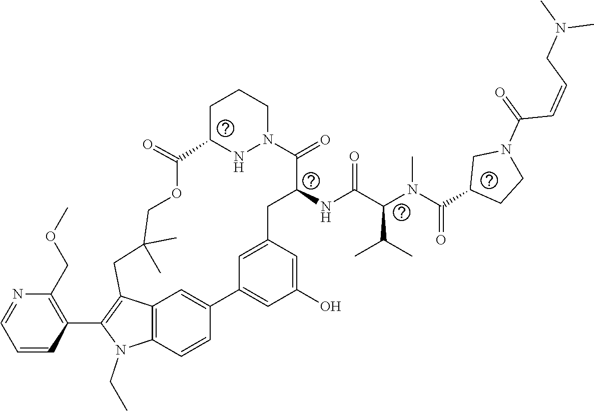 |
| A165 | 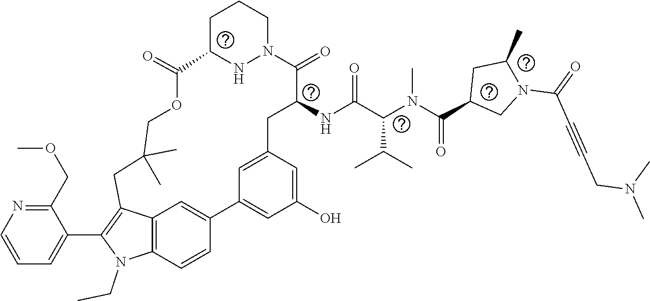 [SMILES: CCn1c(-c2cccnc2COC)c2c3cc(ccc31)-c1cc(O)cc(c1)C[C@H](NC(=O)[C@@H](C(C)C)N(C)C(=O)[C@H]1C[C@@H](C)N(C(=O)C#CCN(C)C)C1)C(=O)N1CCC[C@H](N1)C(=O)OCC(C)(C)C2] |
| A166 | 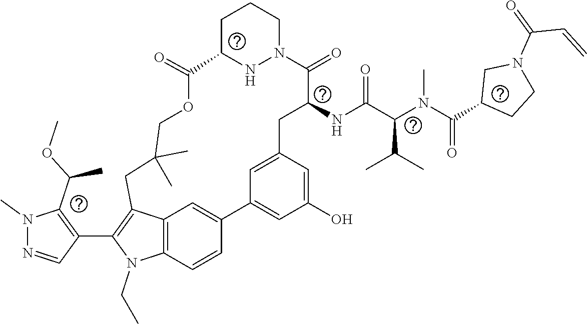 [SMILES: C=CC(=O)N1CC[C@H](C(=O)N(C)[C@H](C(=O)N[C@H]2Cc3cc(O)cc(c3)-c3ccc4c(c3)c(c(-c3cnn(C)c3[C@H](C)OC)n4CC)CC(C)(C)COC(=O)[C@@H]3CCCN(N3)C2=O)C(C)C)C1] |
| A167 | 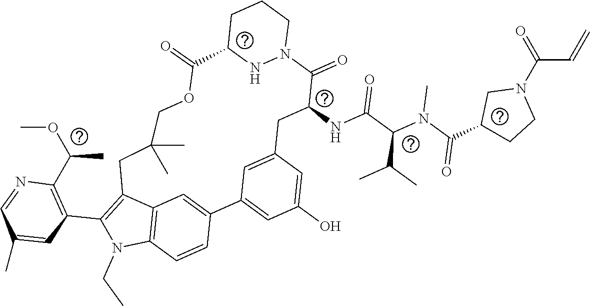 |
| A168 | 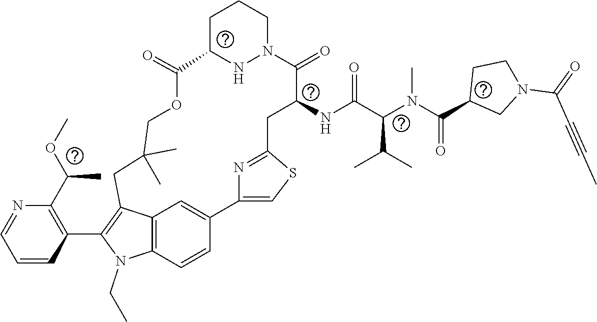 |
| A169 | 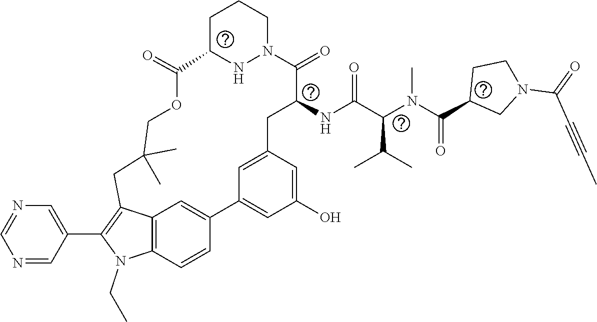 [SMILES: CC#CC(=O)N1CC[C@H](C(=O)N(C)[C@H](C(=O)N[C@H]2Cc3cc(O)cc(c3)-c3ccc4c(c3)c(c(-c3cncnc3)n4CC)CC(C)(C)COC(=O)[C@@H]3CCCN(N3)C2=O)C(C)C)C1] |
| A170 | 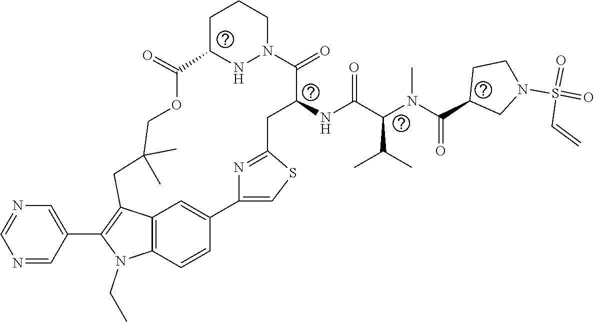 [SMILES: C=CS(=O)(=O)N1CC[C@H](C(=O)N(C)[C@H](C(=O)N[C@H]2Cc3nc(cs3)-c3ccc4c(c3)c(c(-c3cncnc3)n4CC)CC(C)(C)COC(=O)[C@@H]3CCCN(N3)C2=O)C(C)C)C1] |
| A171 | 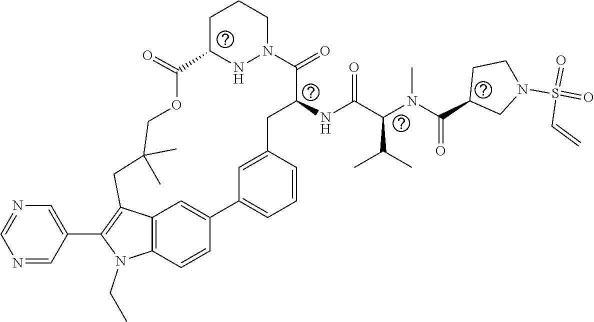 [SMILES: C=CS(=O)(=O)N1CC[C@H](C(=O)N(C)[C@H](C(=O)N[C@H]2Cc3cccc(c3)-c3ccc4c(c3)c(c(-c3cncnc3)n4CC)CC(C)(C)COC(=O)[C@@H]3CCCN(N3)C2=O)C(C)C)C1] |
| A172 | 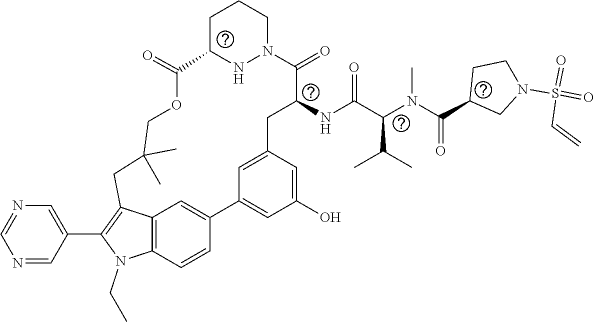 [SMILES: C=CS(=O)(=O)N1CC[C@H](C(=O)N(C)[C@H](C(=O)N[C@H]2Cc3cc(O)cc(c3)-c3ccc4c(c3)c(c(-c3cncnc3)n4CC)CC(C)(C)COC(=O)[C@@H]3CCCN(N3)C2=O)C(C)C)C1] |
| A173 | 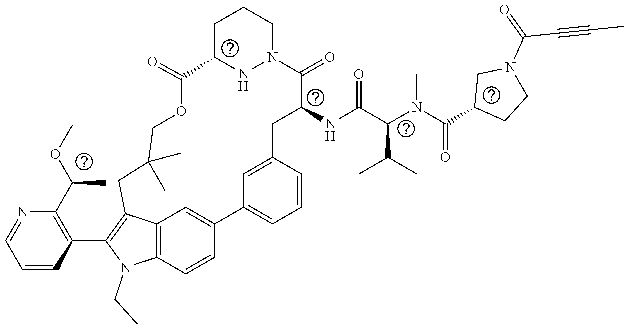 |
| A174 | 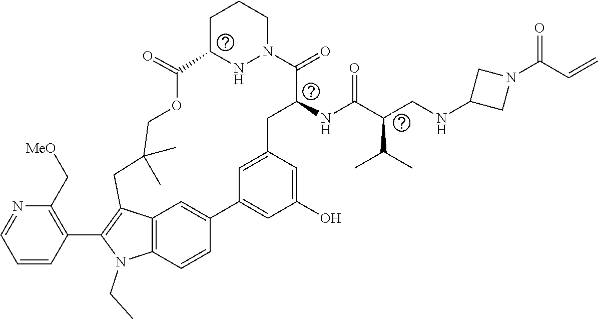 [SMILES: C=CC(=O)N1CC(NC[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A175 | 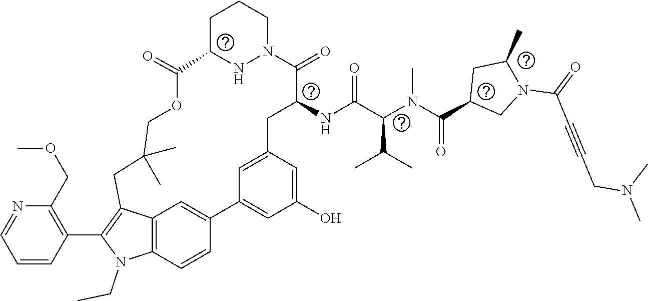 [SMILES: CCn1c(-c2cccnc2COC)c2c3cc(ccc31)-c1cc(O)cc(c1)C[C@H](NC(=O)[C@H](C(C)C)N(C)C(=O)[C@H]1C[C@@H](C)N(C(=O)C#CCN(C)C)C1)C(=O)N1CCC[C@H](N1)C(=O)OCC(C)(C)C2] |
| A176 | 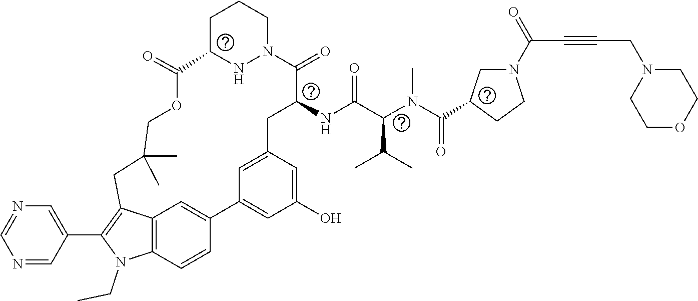 [SMILES: CCn1c(-c2cncnc2)c2c3cc(ccc31)-c1cc(O)cc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A177 | 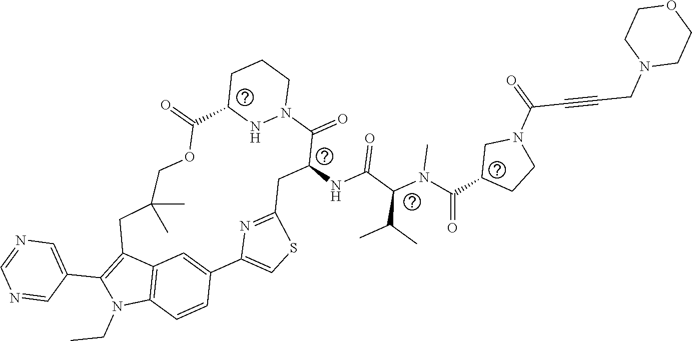 [SMILES: CCn1c(-c2cncnc2)c2c3cc(ccc31)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A178 | 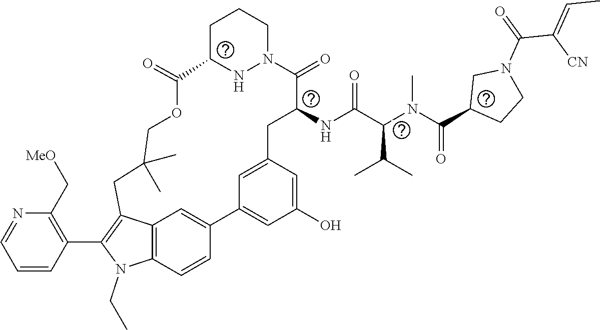 [SMILES: C/C=C(\C#N)C(=O)N1CC[C@@H](C(=O)N(C)[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A179 | 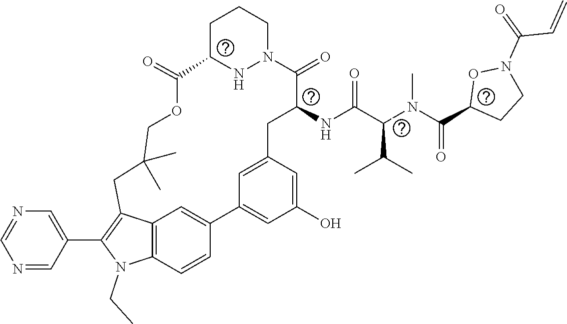 [SMILES: C=CC(=O)N1CC[C@@H](C(=O)N(C)[C@H](C(=O)N[C@H]2Cc3cc(O)cc(c3)-c3ccc4c(c3)c(c(-c3cncnc3)n4CC)CC(C)(C)COC(=O)[C@@H]3CCCN(N3)C2=O)C(C)C)O1] |
| A180 | 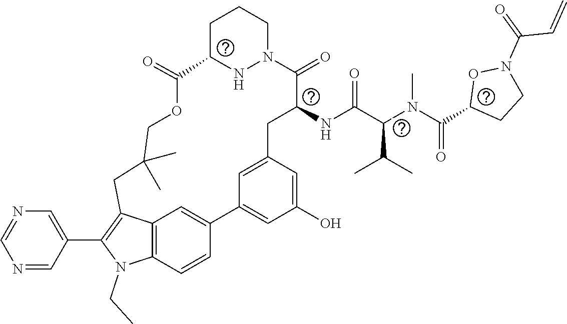 [SMILES: C=CC(=O)N1CC[C@H](C(=O)N(C)[C@H](C(=O)N[C@H]2Cc3cc(O)cc(c3)-c3ccc4c(c3)c(c(-c3cncnc3)n4CC)CC(C)(C)COC(=O)[C@@H]3CCCN(N3)C2=O)C(C)C)O1] |
| A181 | 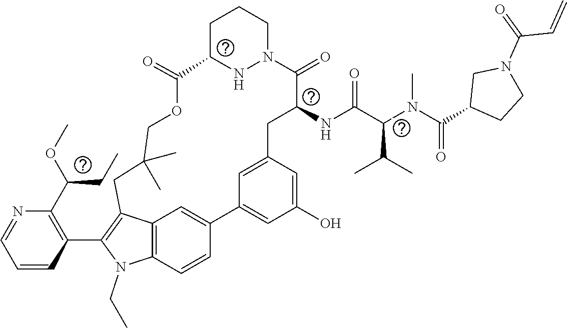 |
| A182 | 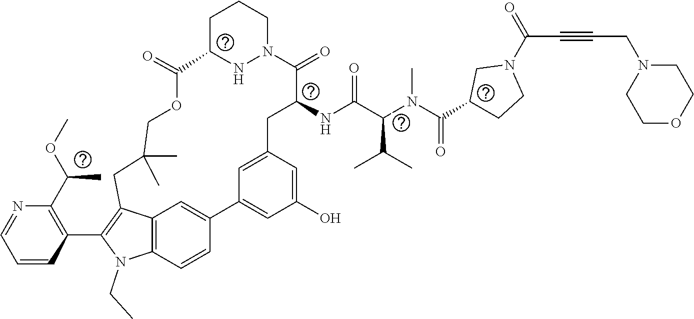 |
| A183 | 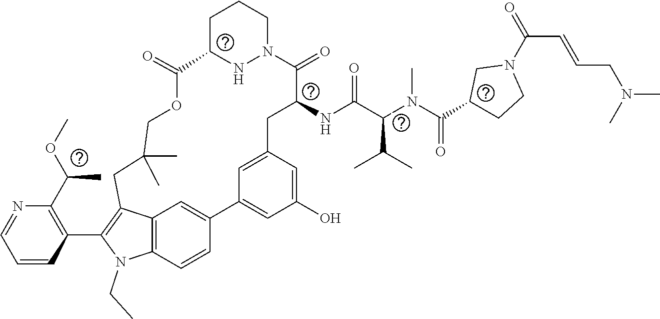 |
| A184 | 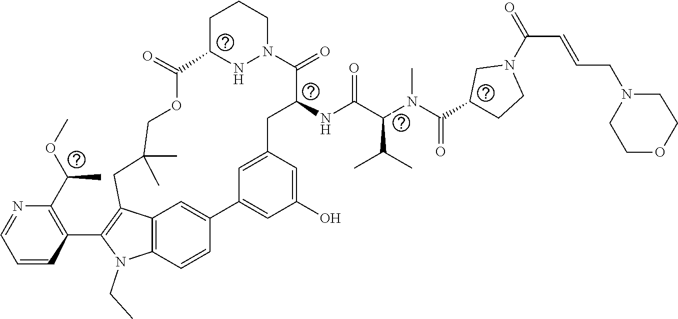 |
| A185 | 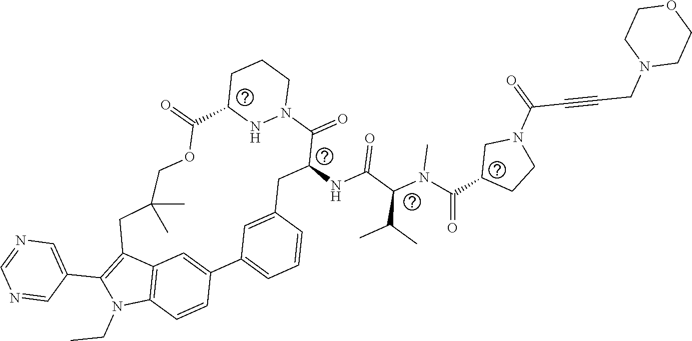 [SMILES: CCn1c(-c2cncnc2)c2c3cc(ccc31)-c1cccc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A186 | 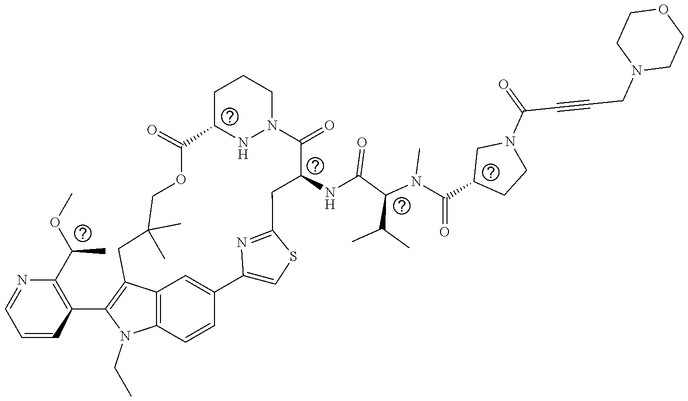 |
| A187 | 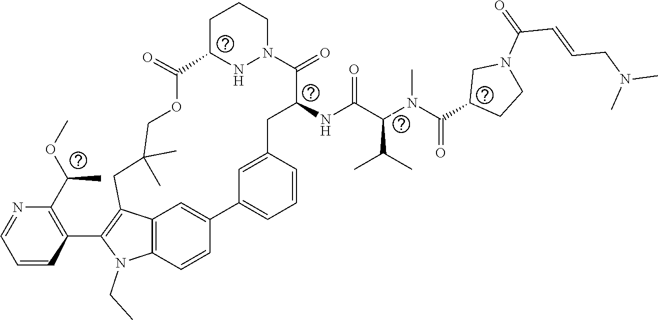 |
| A188 | 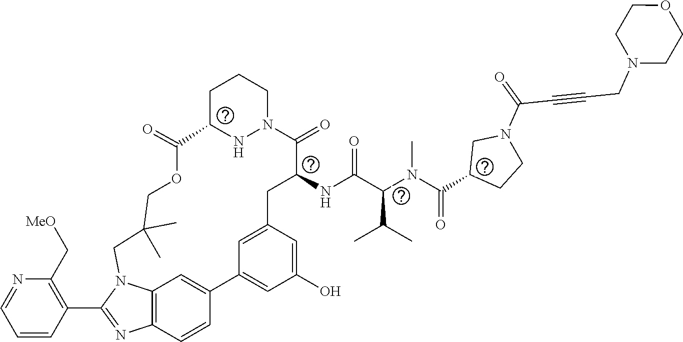 [SMILES: COCc1ncccc1-c1nc2ccc3cc2n1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| A189 | 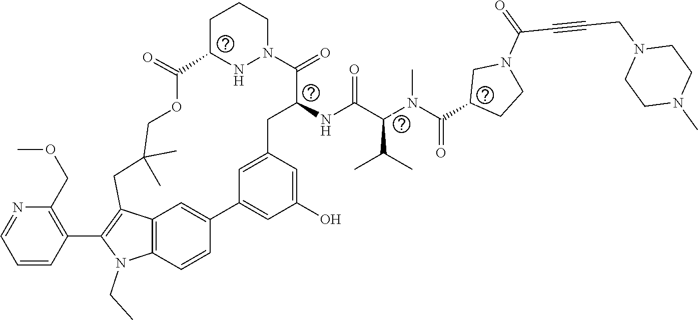 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3CCN(C)CC3)C1)C(=O)N1CCC[C@H](N1)C(=O)OCC(C)(C)C2] |
| A190 | 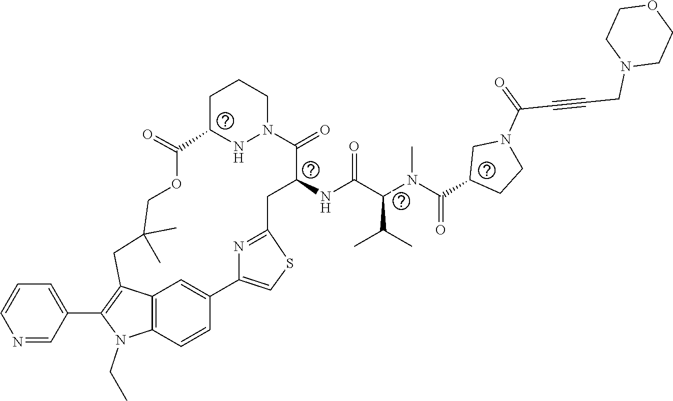 [SMILES: CCn1c(-c2cccnc2)c2c3cc(ccc31)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A191 | 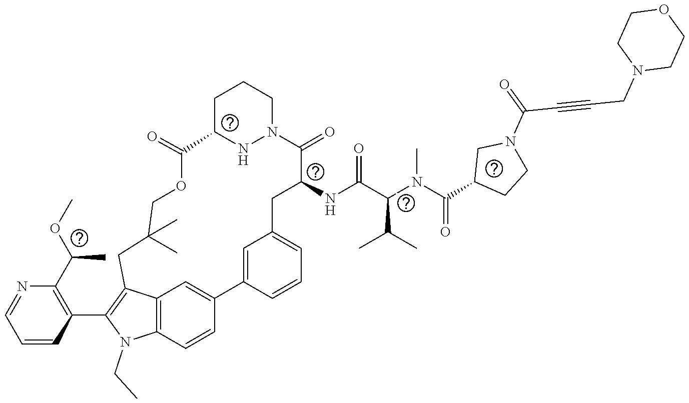 |
| A192 | 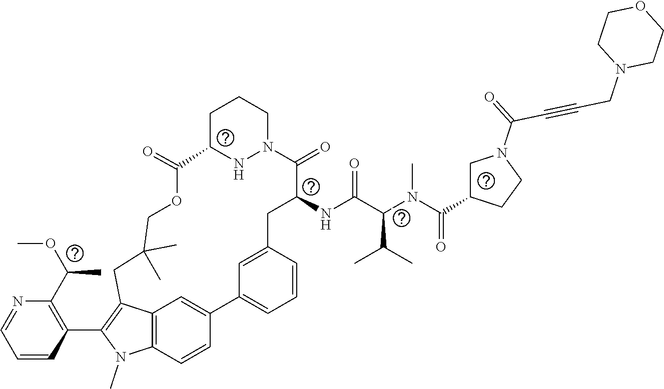 |
| A193 | 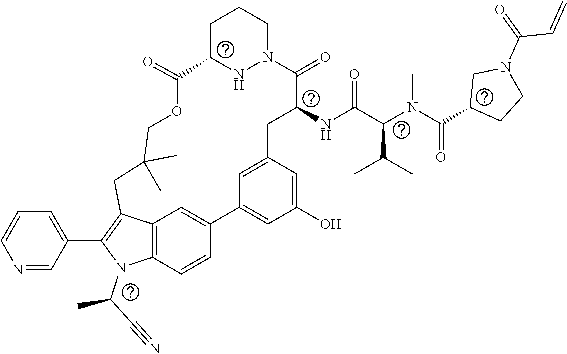 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4[C@H](C)C#N)CC(C)(C)COC(=O)[C@@H]3CCCN(N3)C2=O)C(C)C)C1] |
| A194 | 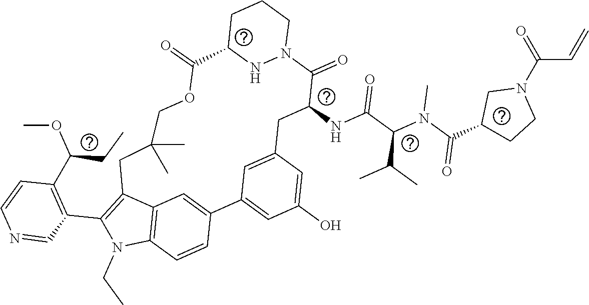 |
| A195 | 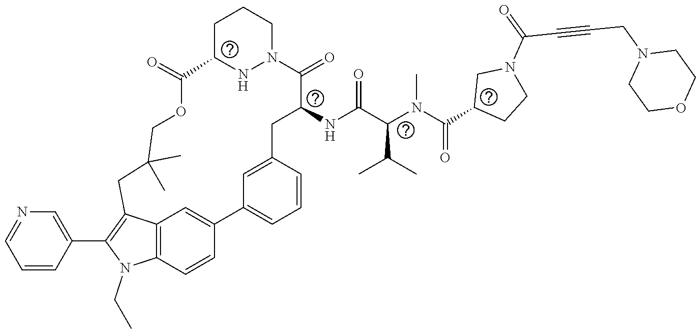 [SMILES: CCn1c(-c2cccnc2)c2c3cc(ccc31)-c1cccc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A196 | 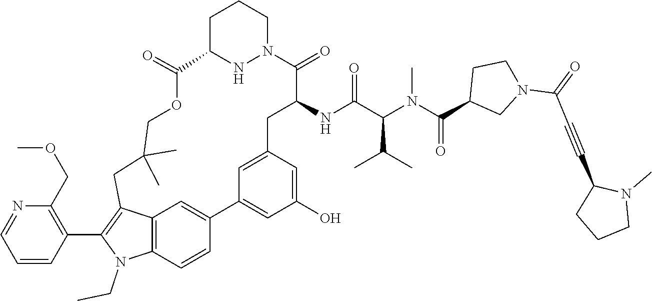 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| A197 | 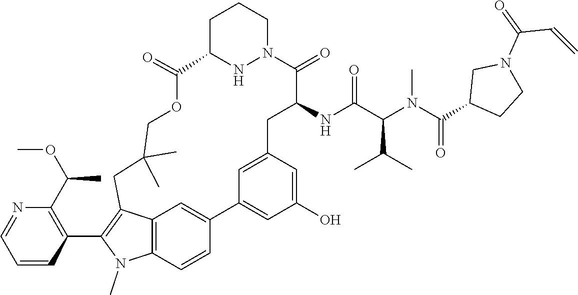 |
| A198 | 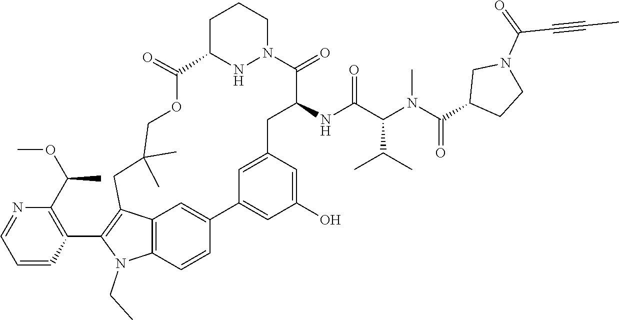 |
| A199 | 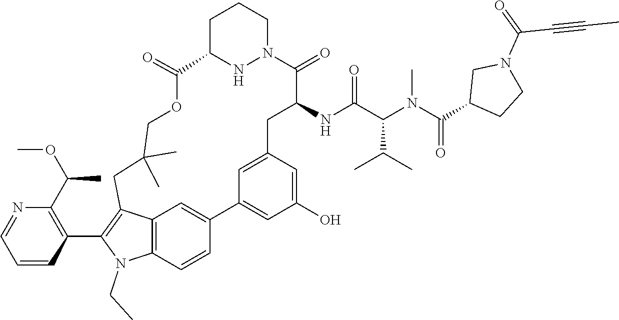 |
| A200 | 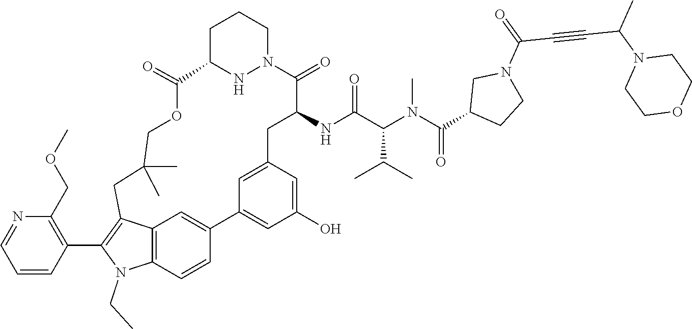 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CC(C)N3CCOCC3)C1)C(=O)N1CCC[C@H](N1)C(=O)OCC(C)(C)C2] |
| A201 | 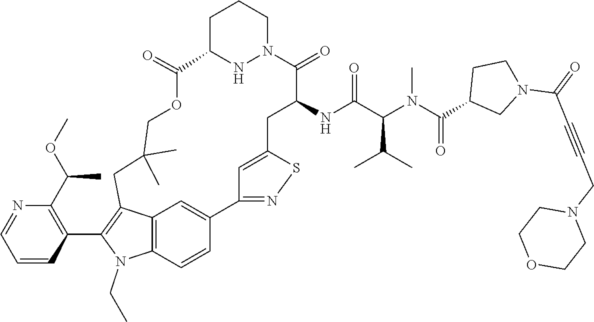 |
| A202 | 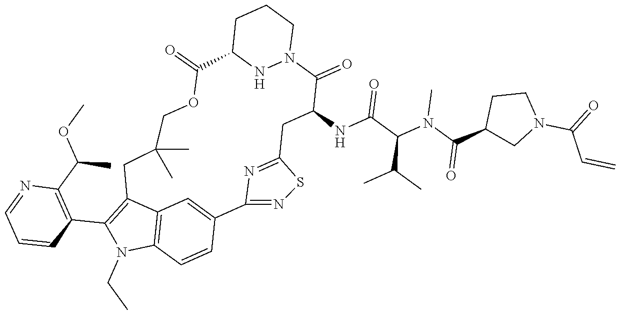 |
| A203 | 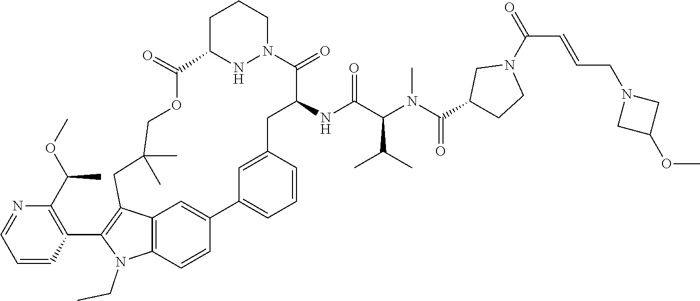 |
| A204 | 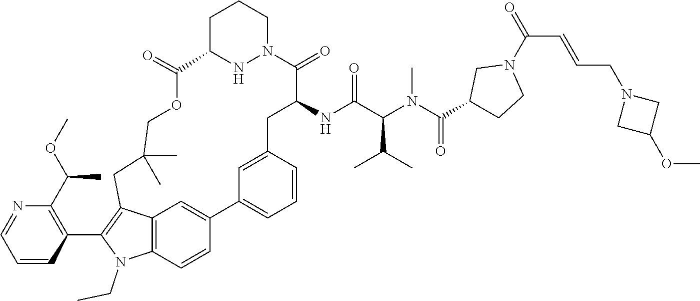 |
| A205 | 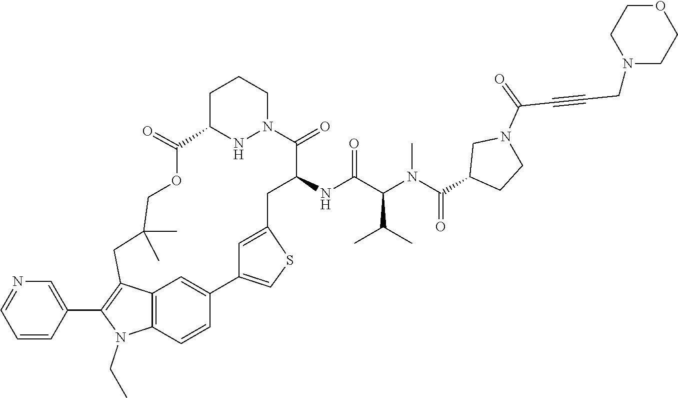 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A206 | 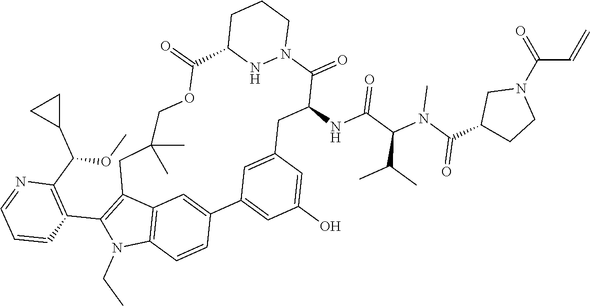 |
| A207 | 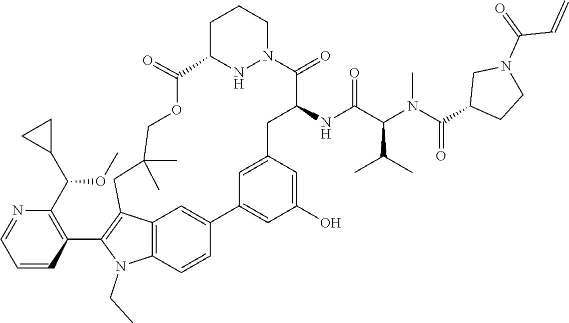 |
| A208 | 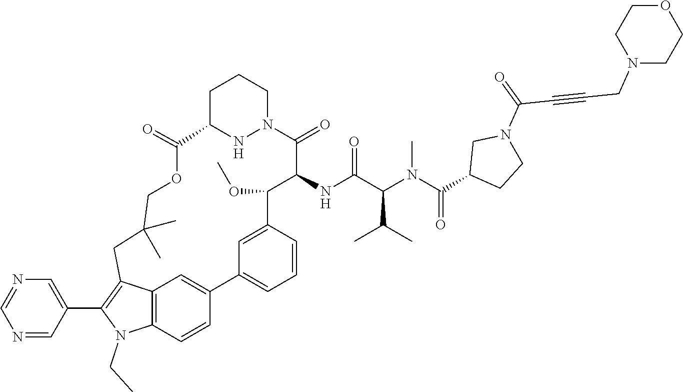 [SMILES: CCn1c(-c2cncnc2)c2c3cc(ccc31)-c1cccc(c1)[C@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A209 | 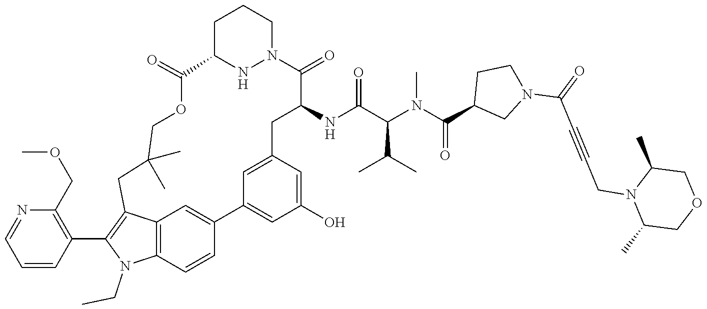 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@@H](C)COC[C@@H]3C)C1)C(=O)N1CCC[C@H](N1)C(=O)OCC(C)(C)C2] |
| A210 | 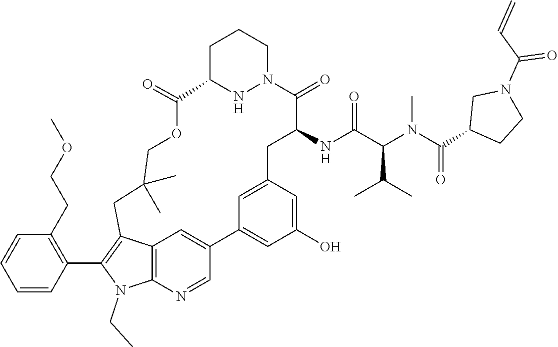 [SMILES: C=CC(=O)N1CC[C@H](C(=O)N(C)[C@H](C(=O)N[C@H]2Cc3cc(O)cc(c3)-c3cnc4c(c3)c(c(-c3ccccc3CCOC)n4CC)CC(C)(C)COC(=O)[C@@H]3CCCN(N3)C2=O)C(C)C)C1] |
| A211 | 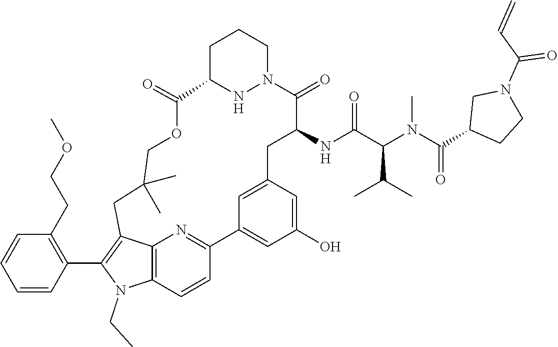 [SMILES: C=CC(=O)N1CC[C@H](C(=O)N(C)[C@H](C(=O)N[C@H]2Cc3cc(O)cc(c3)-c3ccc4c(n3)c(c(-c3ccccc3CCOC)n4CC)CC(C)(C)COC(=O)[C@@H]3CCCN(N3)C2=O)C(C)C)C1] |
| A212 | 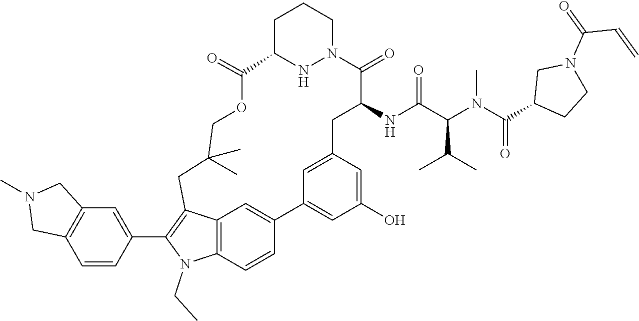 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CN(C)C5)n4CC)CC(C)(C)COC(=O)[C@@H]3CCCN(N3)C2=O)C(C)C)C1] |
| A213 | 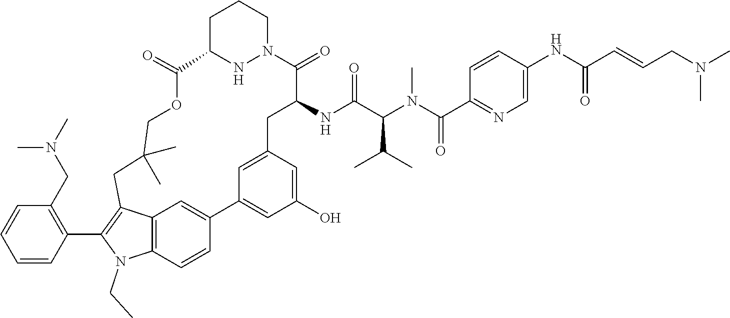 [SMILES: CCn1c(-c2ccccc2CN(C)C)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| A214 | 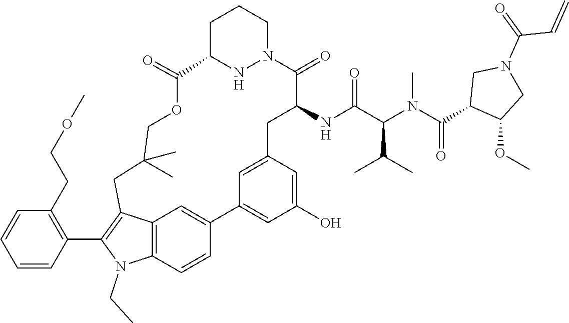 [SMILES: C=CC(=O)N1C[C@H](OC)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| A215 | 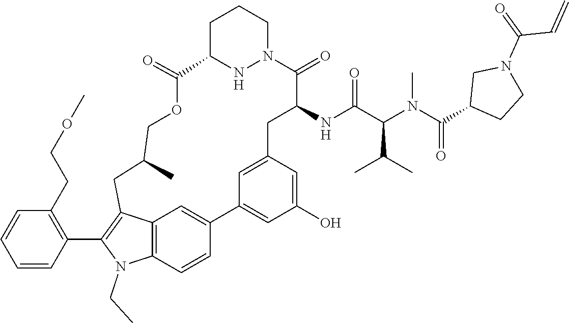 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)C[C@H](C)COC(=O)[C@@H]3CCCN(N3)C2=O)C(C)C)C1] |
| A216 | 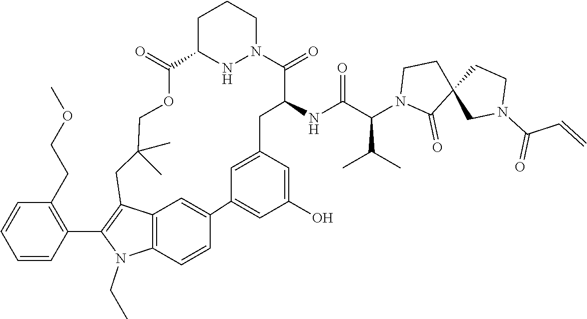 [SMILES: C=CC(=O)N1CC[C@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| A217 | 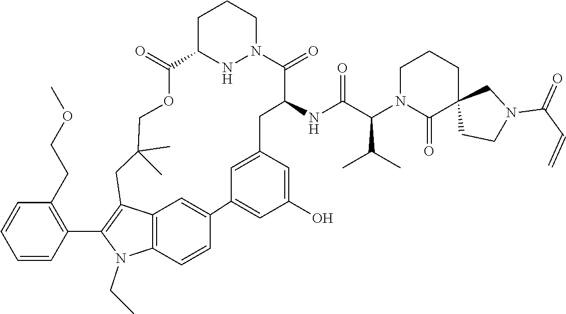 [SMILES: C=CC(=O)N1CC[C@]2(CCCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| A218 | 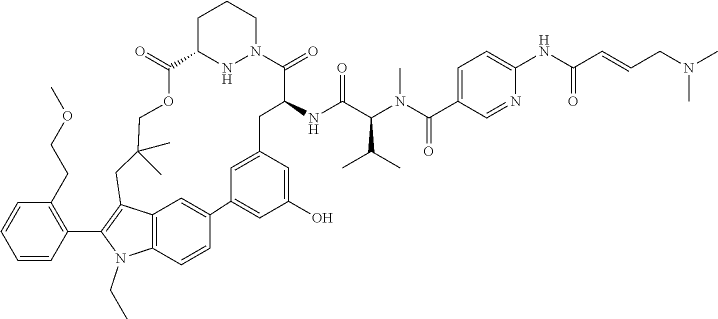 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)nc1)C(=O)N1CCC[C@H](N1)C(=O)OCC(C)(C)C2] |
| A219 | 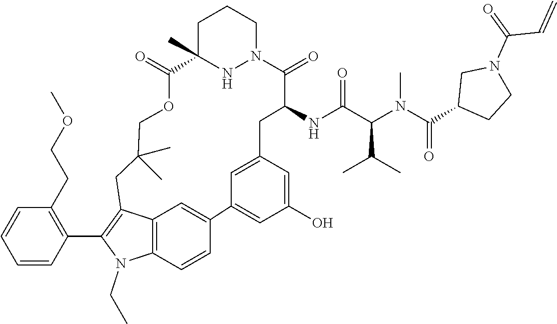 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@]3(C)CCCN(N3)C2=O)C(C)C)C1] |
| A220 | 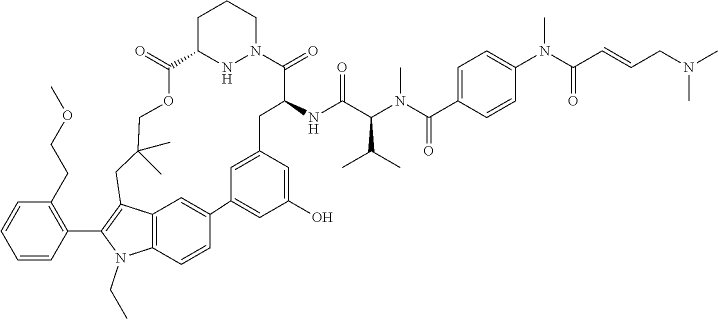 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(N(C)C(=O)/C=C/CN(C)C)cc1)C(=O)N1CCC[C@H](N1)C(=O)OCC(C)(C)C2] |
| A221 | 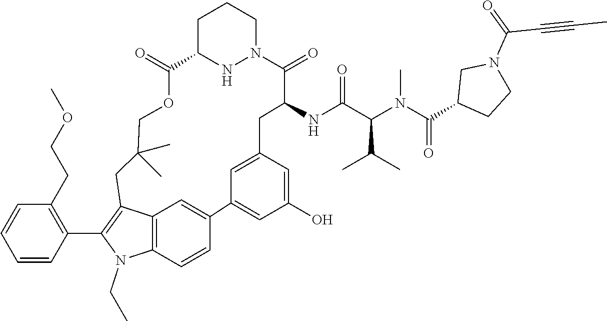 [SMILES: CC#CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| A222 | 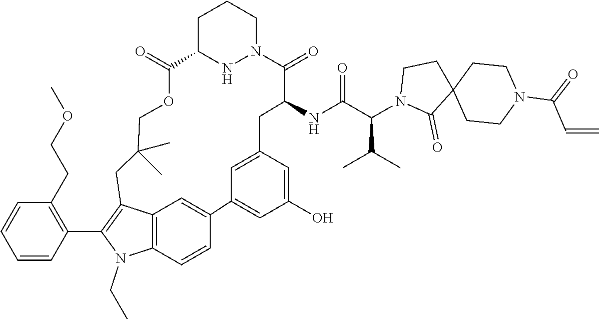 [SMILES: C=CC(=O)N1CCC2(CC1)CCN([C@H](C(=O)N[C@H]1Cc3cc(O)cc(c3)-c3ccc4c(c3)c(c(-c3ccccc3CCOC)n4CC)CC(C)(C)COC(=O)[C@@H]3CCCN(N3)C1=O)C(C)C)C2=O] |
| A223 | 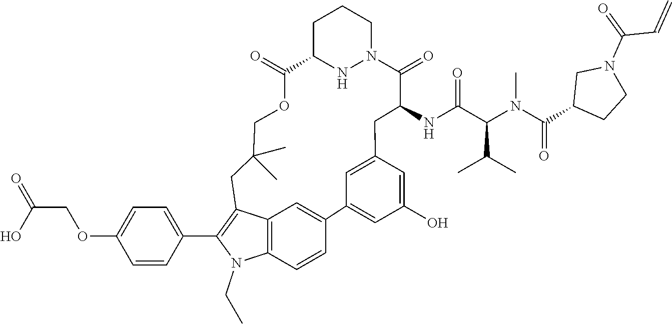 [SMILES: C=CC(=O)N1CC[C@H](C(=O)N(C)[C@H](C(=O)N[C@H]2Cc3cc(O)cc(c3)-c3ccc4c(c3)c(c(-c3ccc(OCC(=O)O)cc3)n4CC)CC(C)(C)COC(=O)[C@@H]3CCCN(N3)C2=O)C(C)C)C1] |
| A224 | 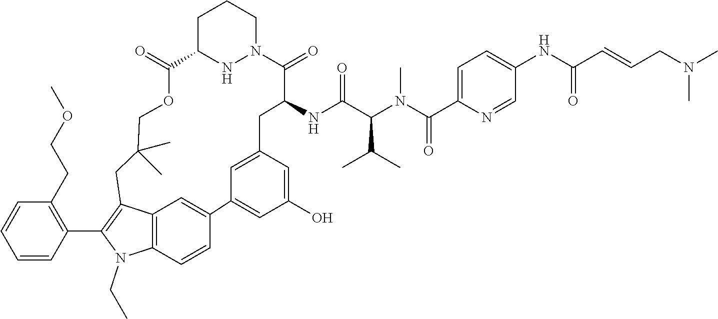 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| A225 | 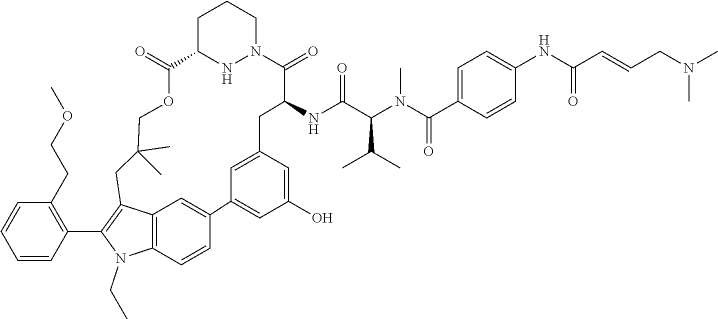 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cc1)C(=O)N1CCC[C@H](N1)C(=O)OCC(C)(C)C2] |
| A226 | 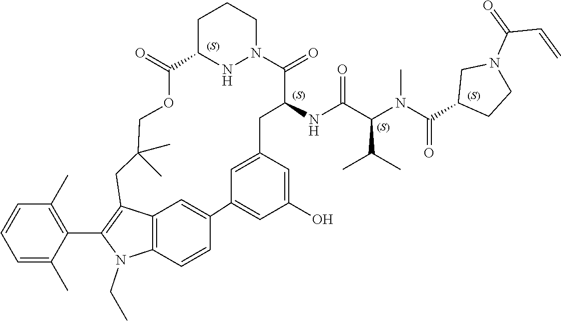 |
| A227 | 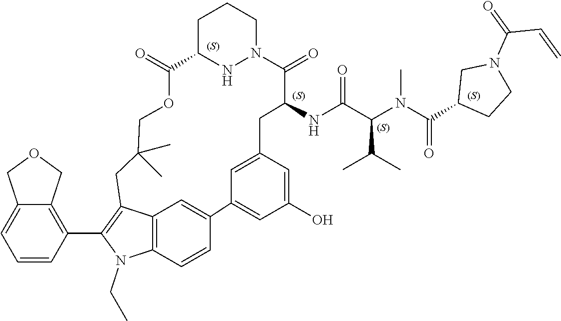 |
| A228 | 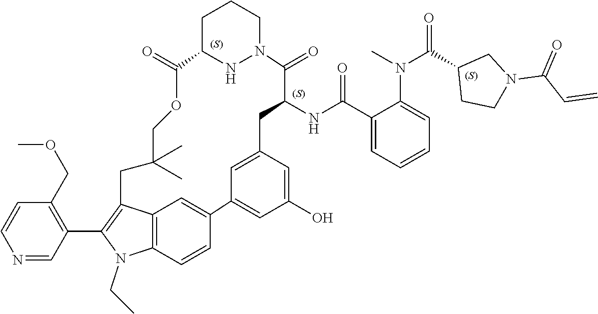 |
| A229 | 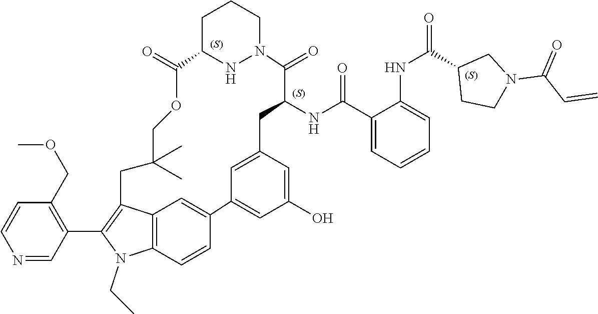 |
| A230 | 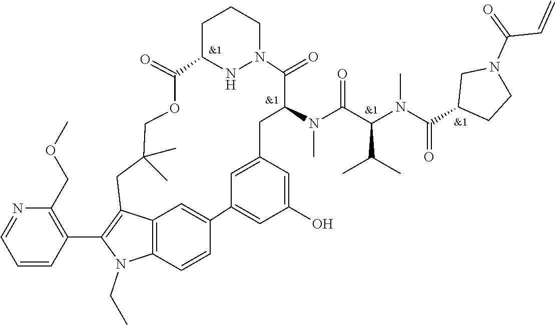 |
| A231 | 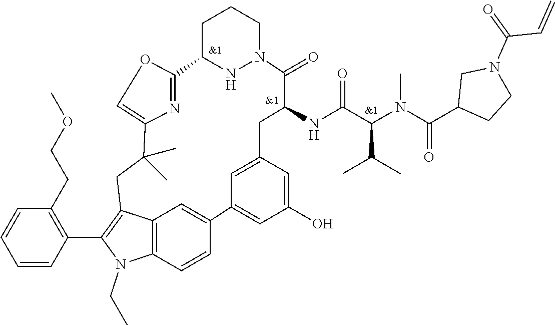 |
| A232 | 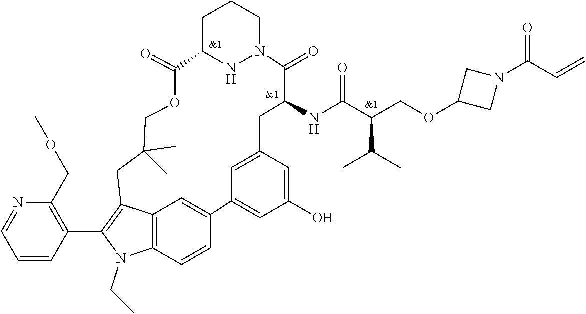 |
| A233 | 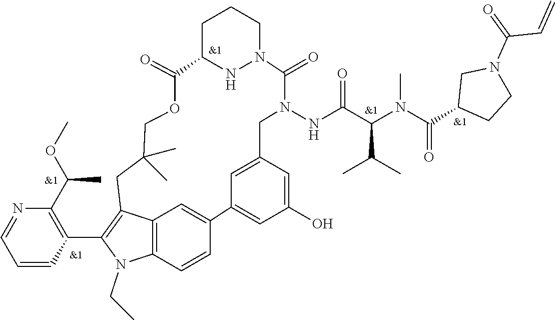 |
| A234 | 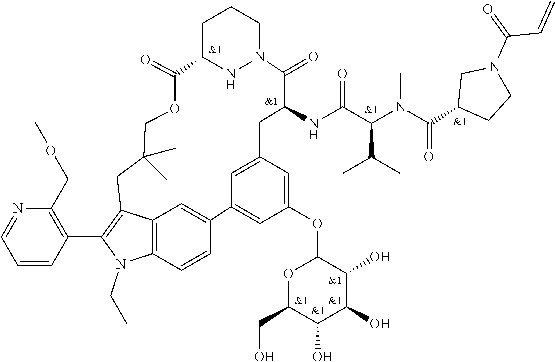 |
| A235 | 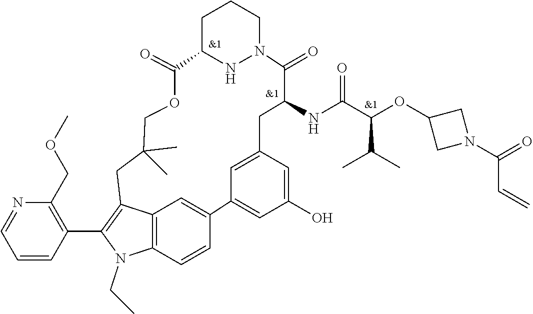 |
| A236 | 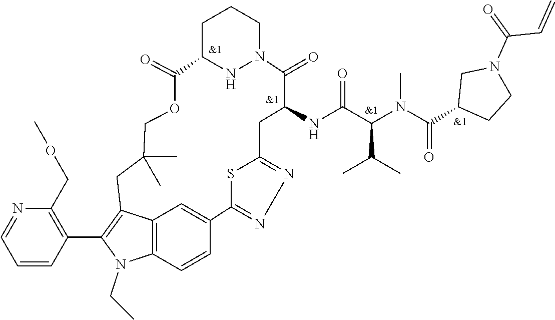 |
| A237 | 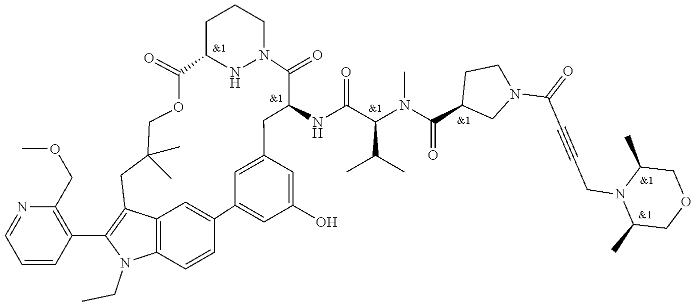 |
| A238 | 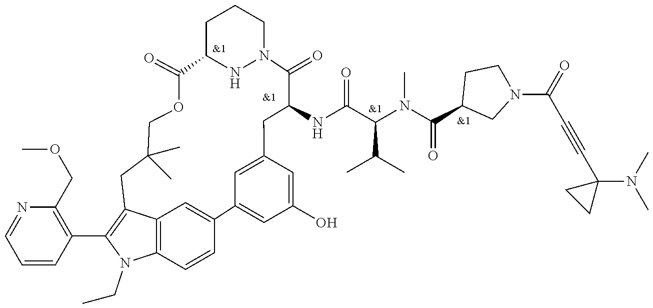 |
| A239 | 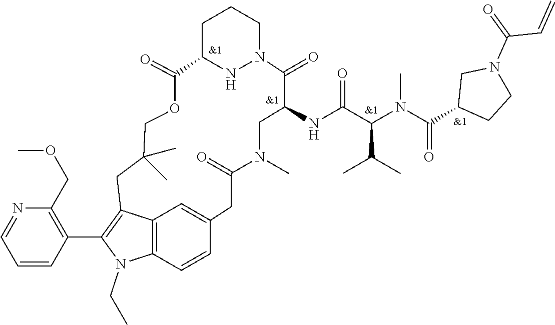 |
| A240 | 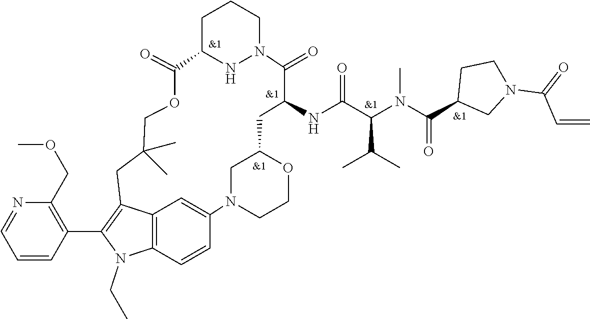 |
| A241 | 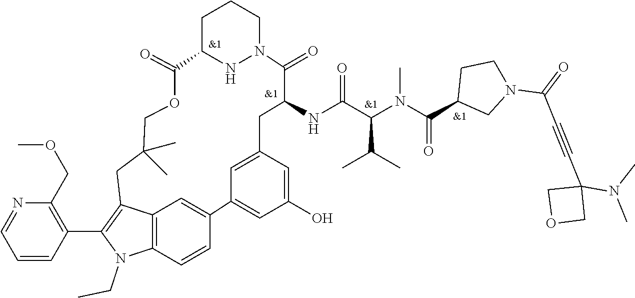 |
| A242 | 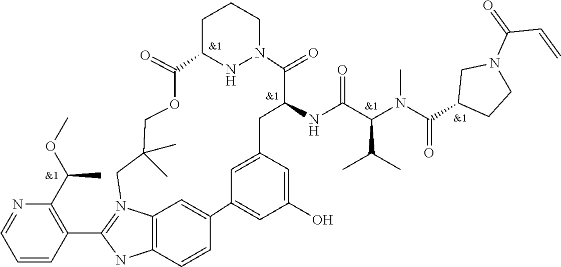 |
| A243 | 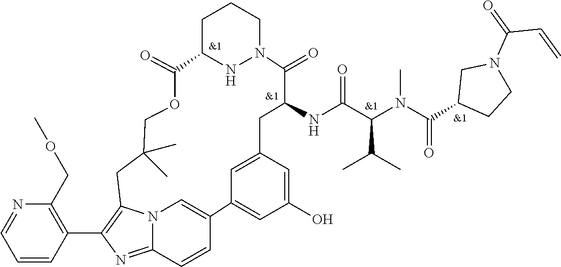 |
| A244 | 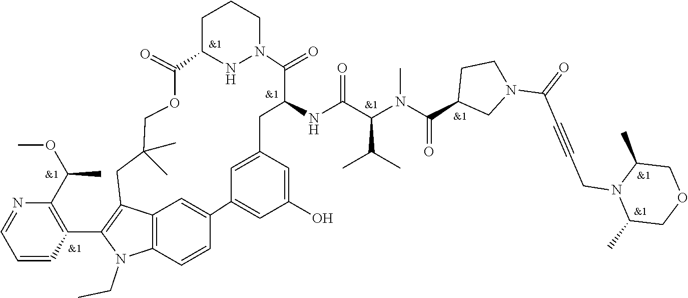 |
| A245 | 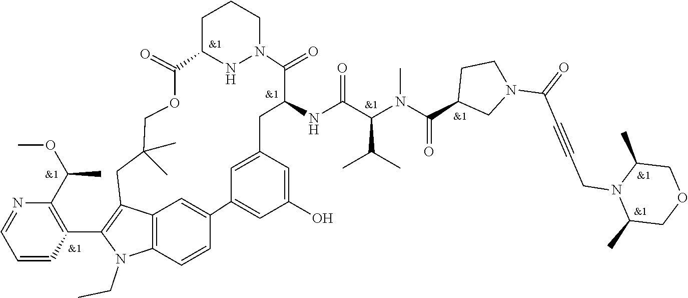 |
| A246 | 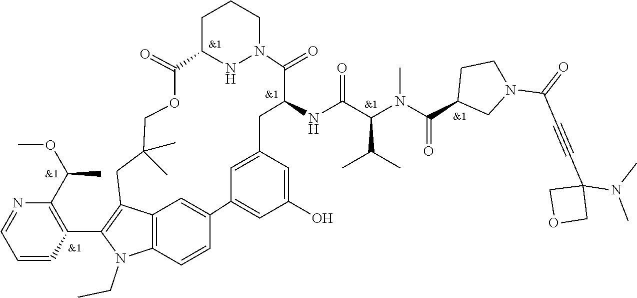 |
| A247 | 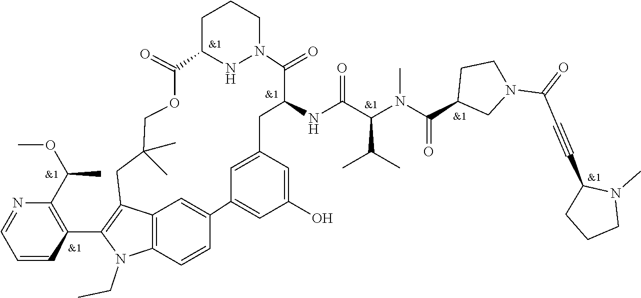 |
| A248 | 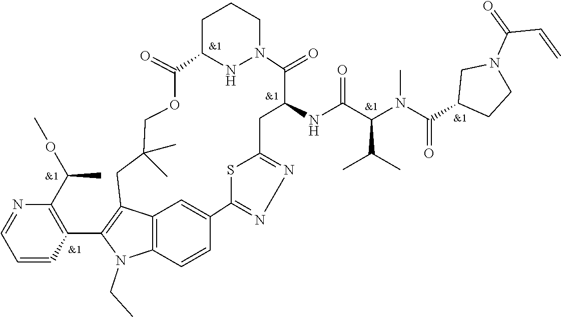 |
| A249 | 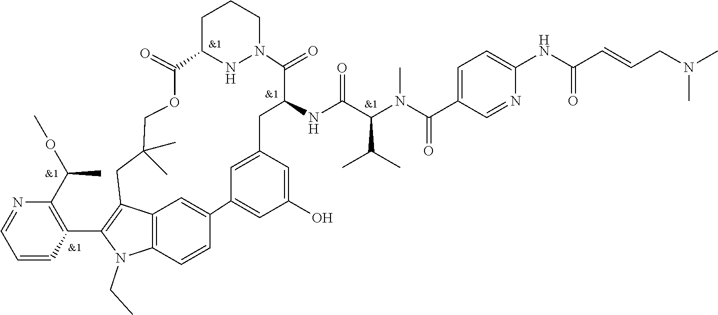 |
| A250 | 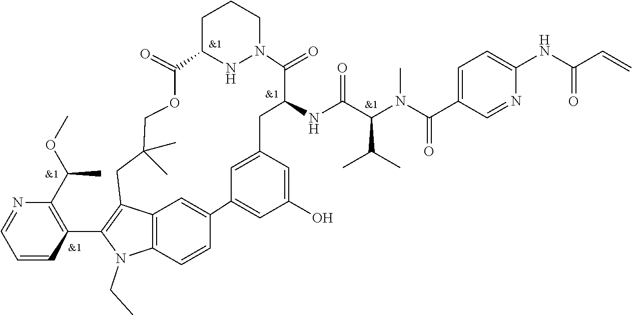 |
| A251 | 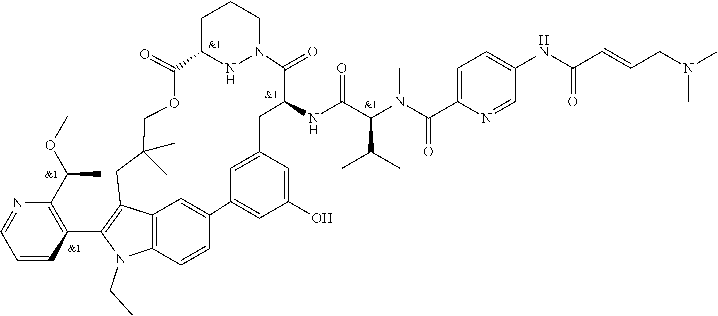 |
| A252 | 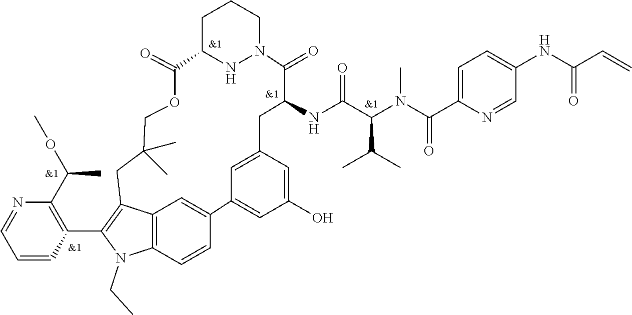 |
| A253 | 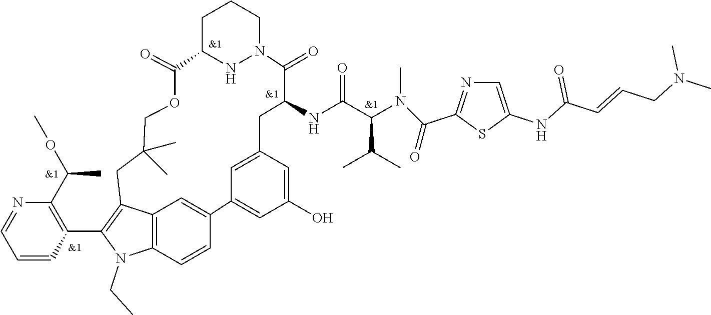 |
| A254 | 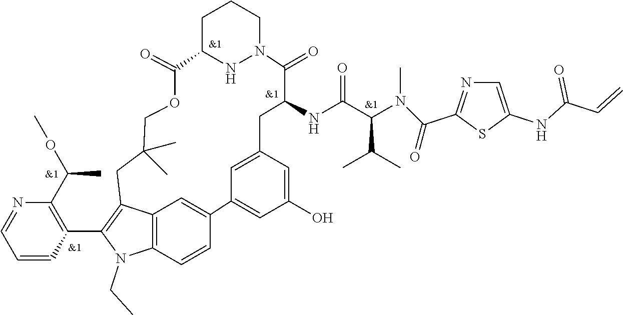 |
| A255 | 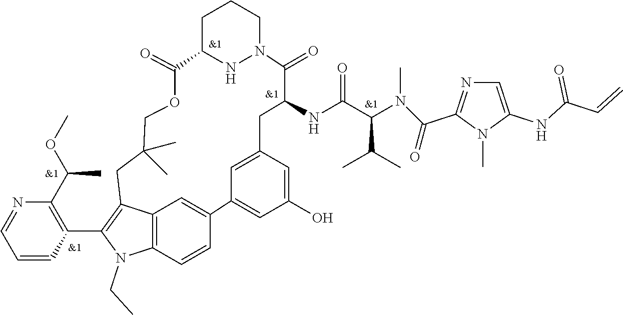 |
| A256 | 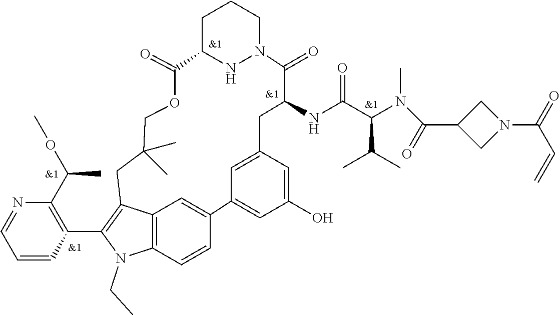 |
| A257 | 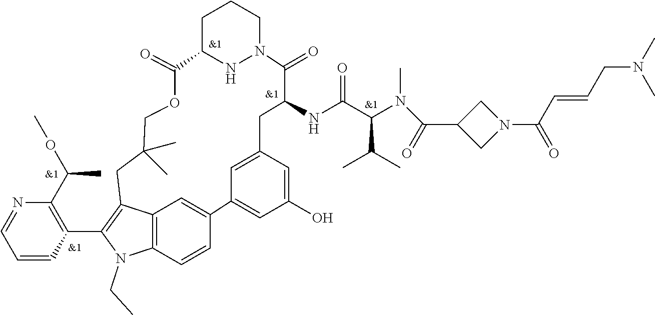 |
| A258 | 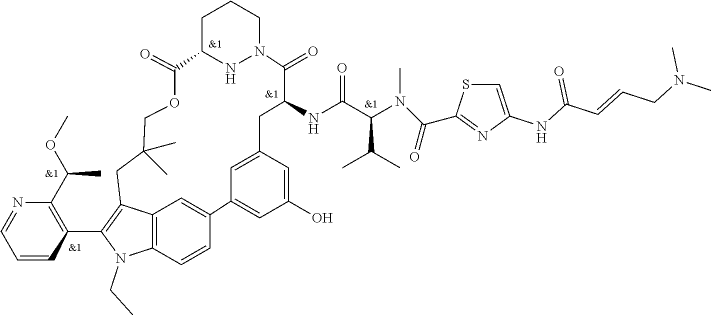 |
| A259 | 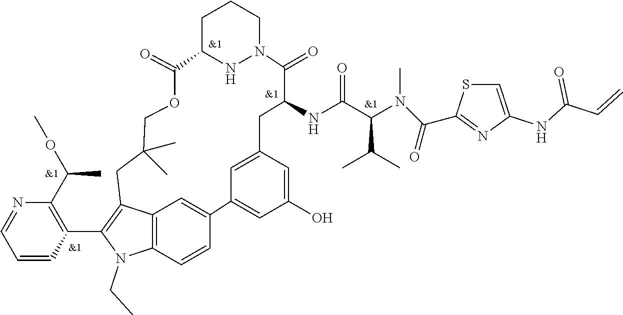 |
| A260 | 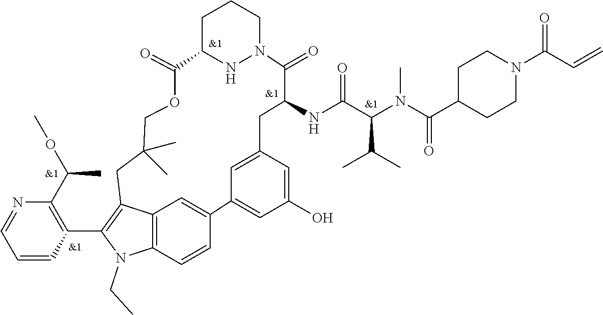 |
| A261 | 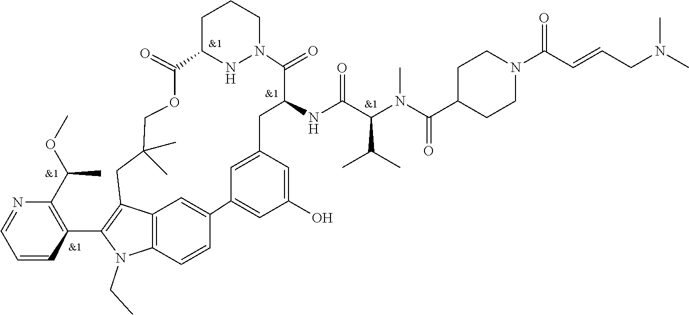 |
| A262 | 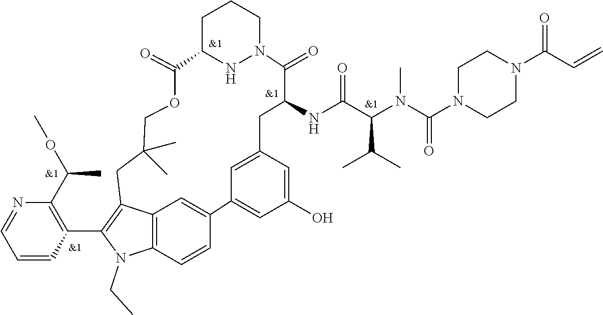 |
| A263 | 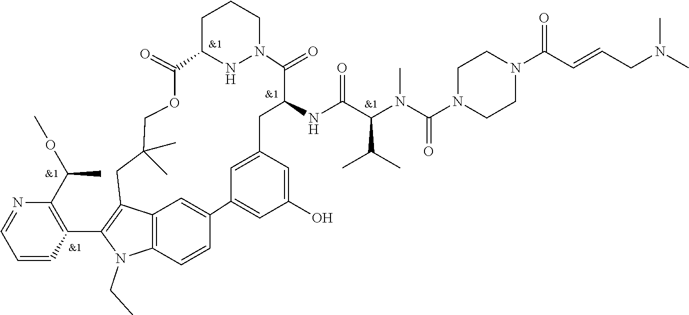 |
| A264 | 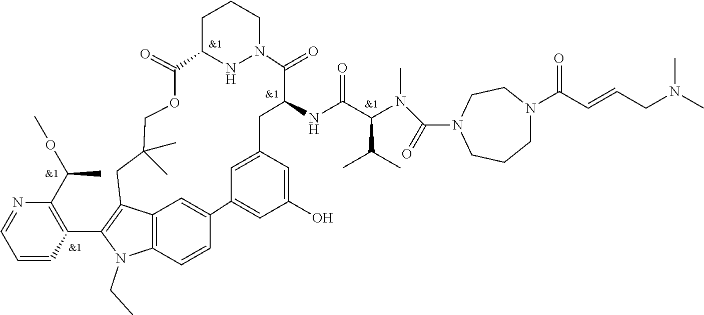 |
| A265 | 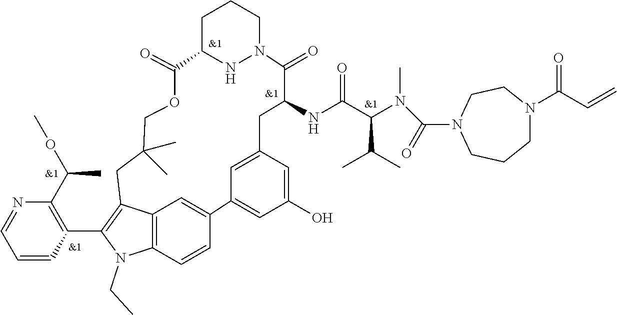 |
| A266 | 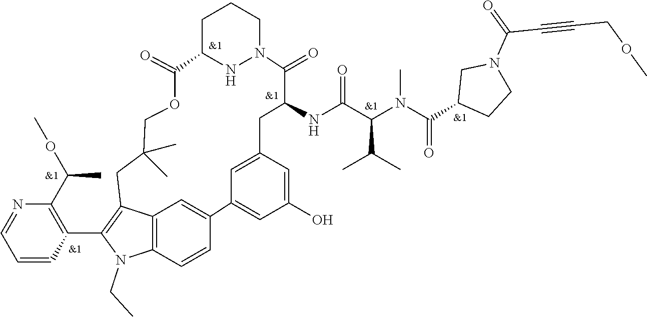 |
| A267 | 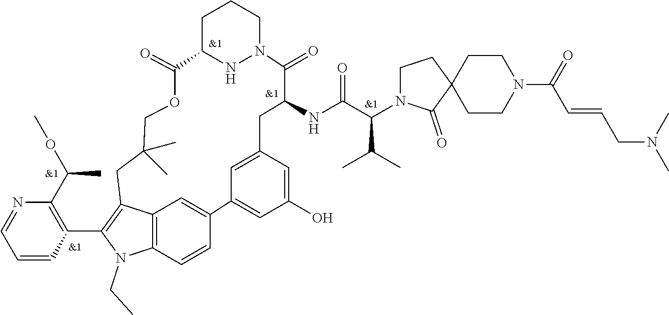 |
| A268 | 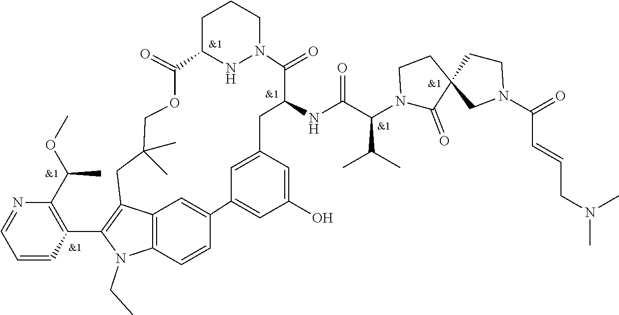 |
| A269 | 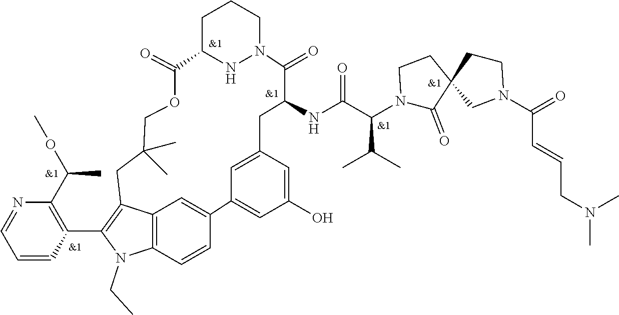 |
| A270 | 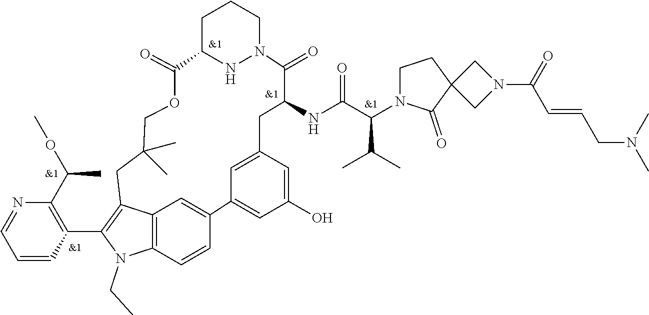 |
| A271 | 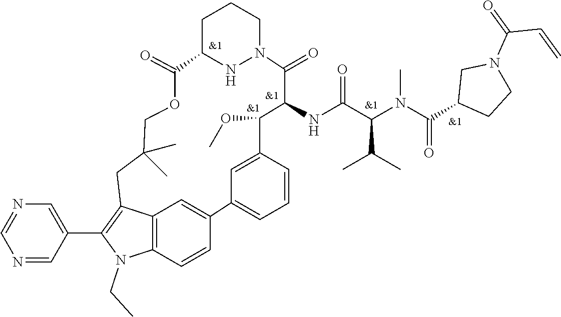 |
| A272 | 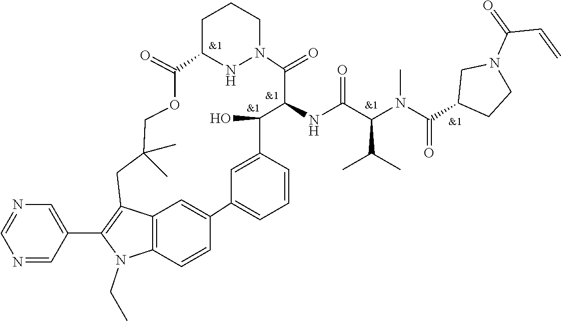 |
| A273 | 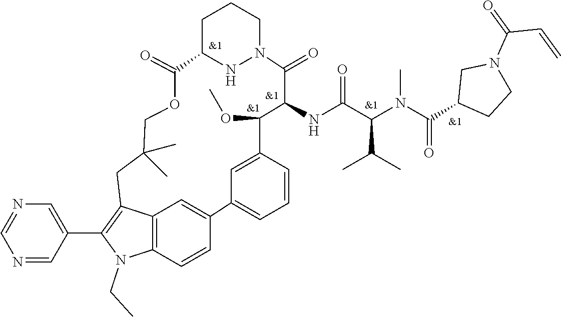 |
| A274 | 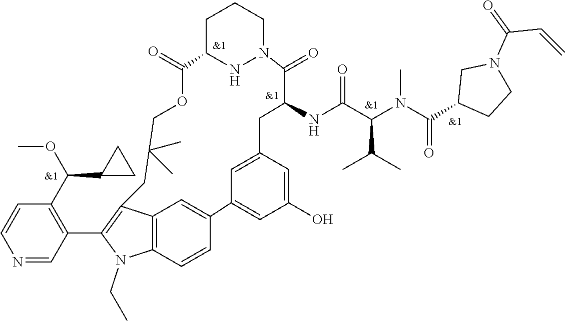 |
| A275 | 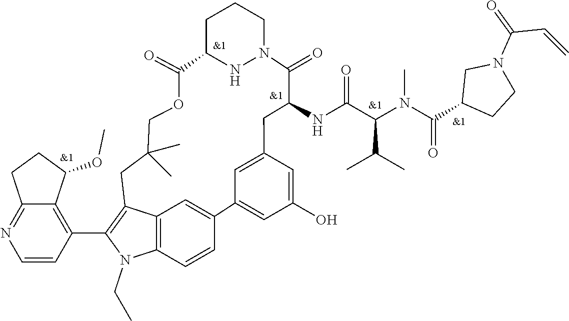 |
| A276 | 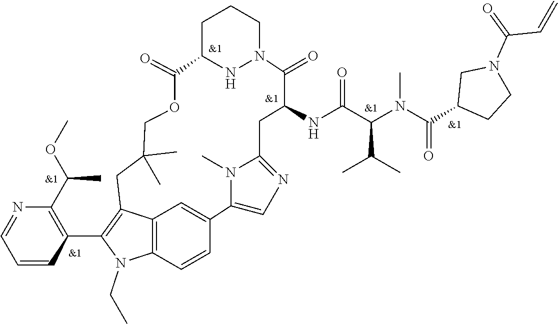 |
| A277 | 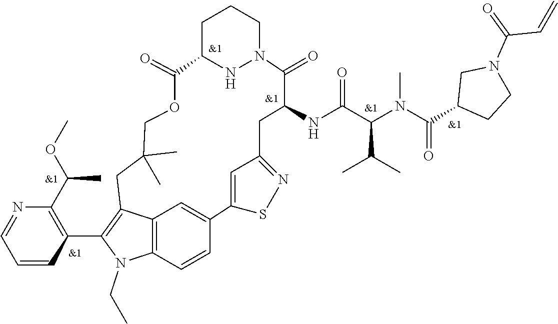 |
| A278 | 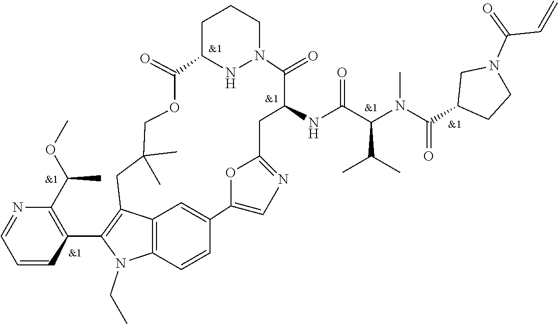 |
| A279 | 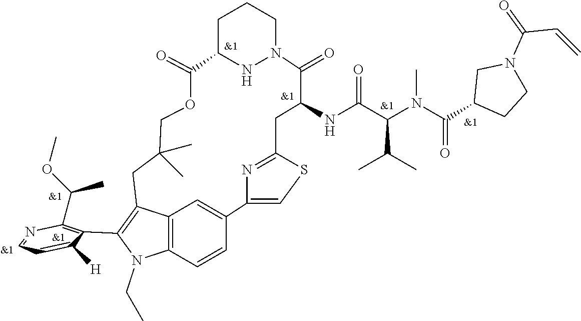 |
| A280 | 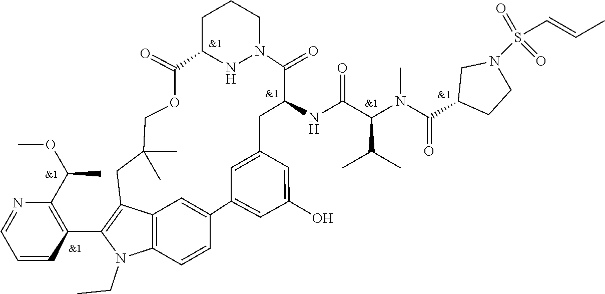 |
| A281 | 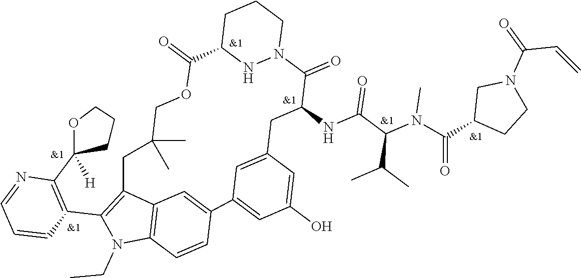 |
| A282 | 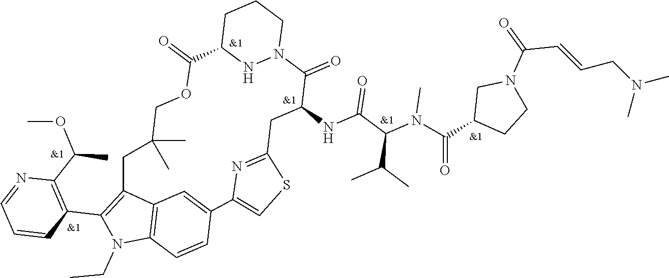 |
| A283 | 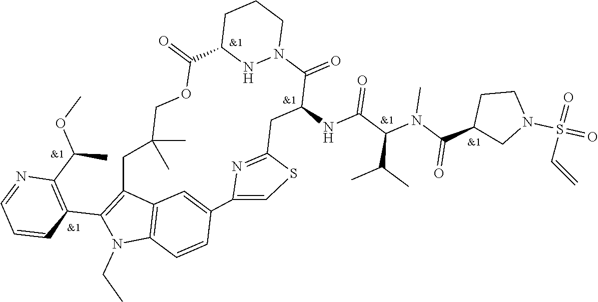 |
| A284 | 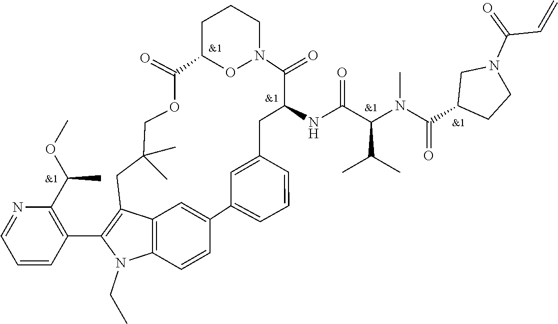 |
| A285 | 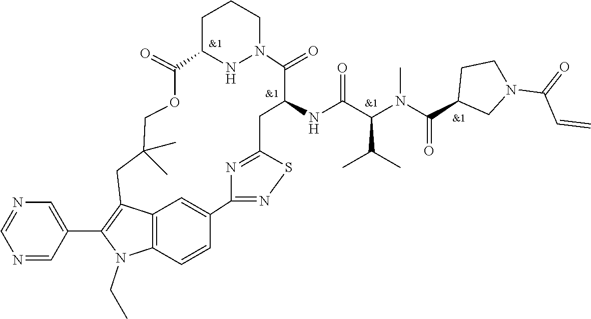 |
| A286 | 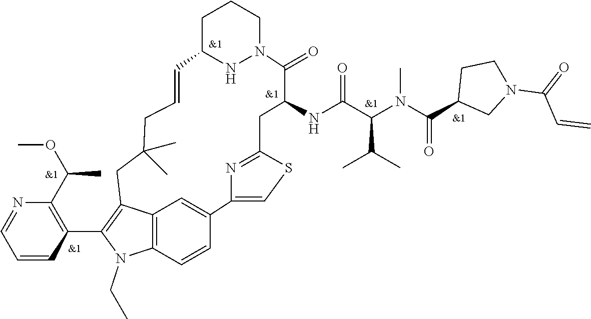 |
| A287 | 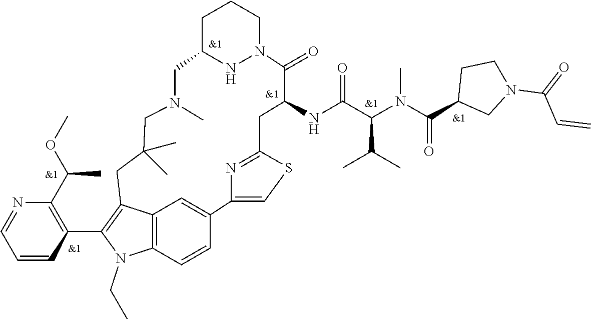 |
| A288 | 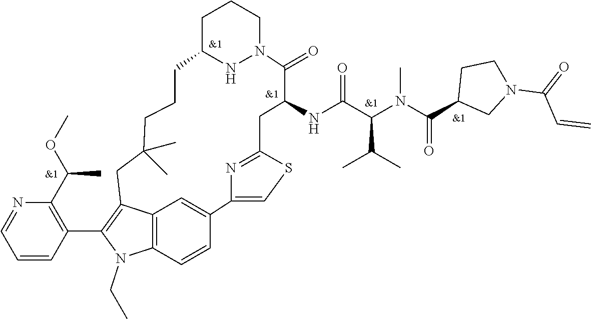 |
| A289 | 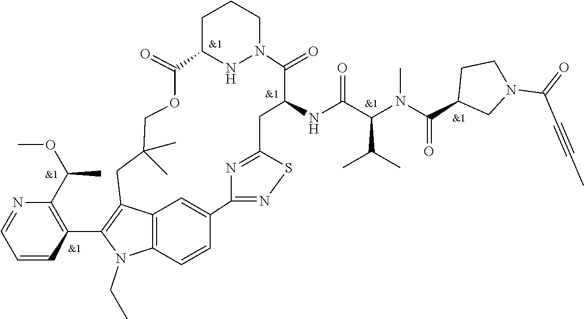 |
| A290 | 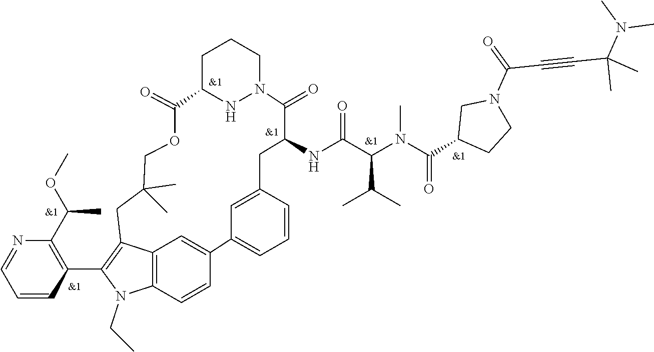 |
| A291 | 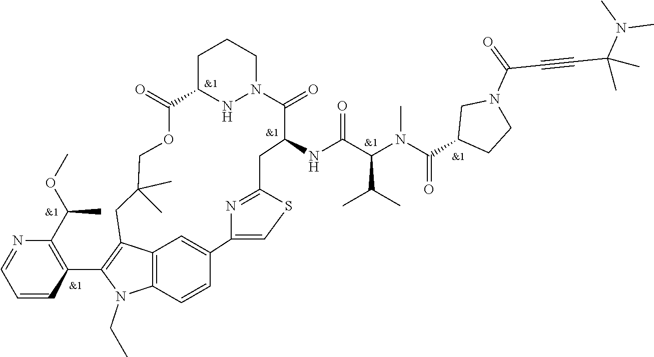 |
| A292 | 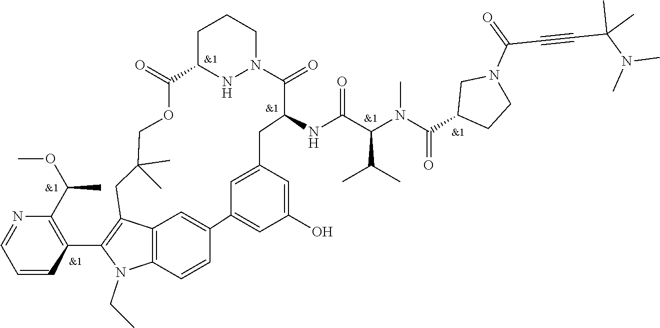 |
| A293 | 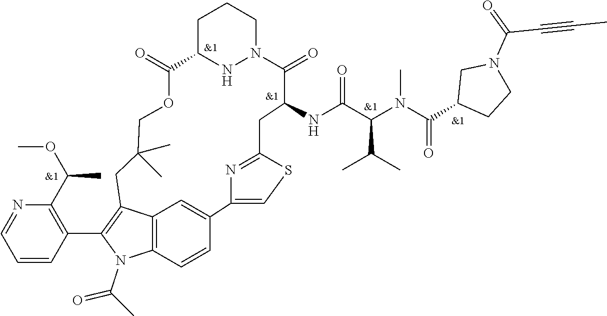 |
| A294 | 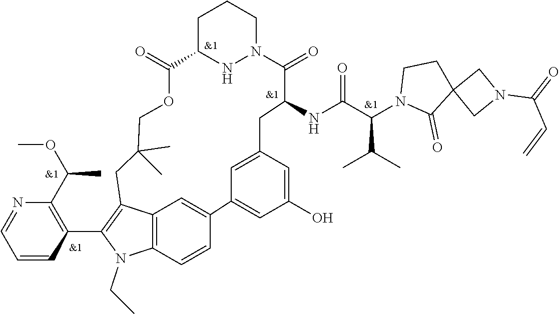 |
| A295 | 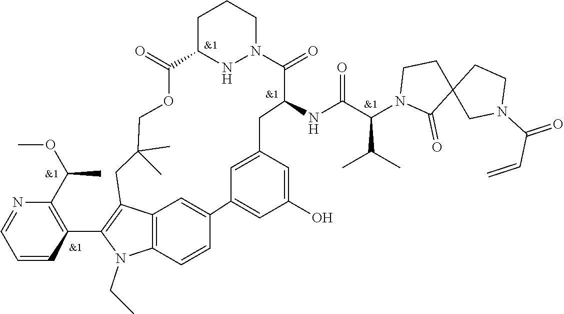 |
| A296 | 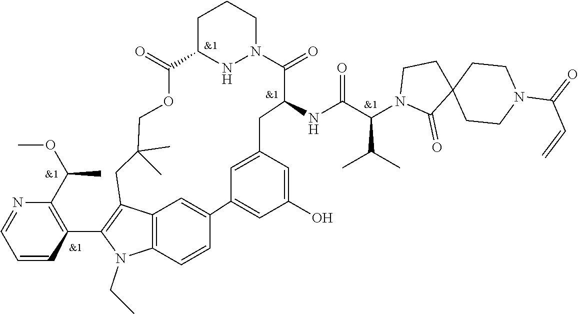 |
| A297 | 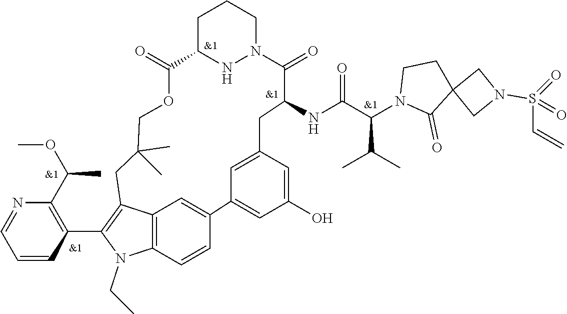 |
| A298 | 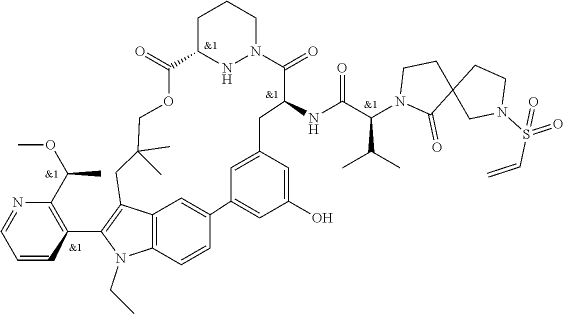 |
| A299 | 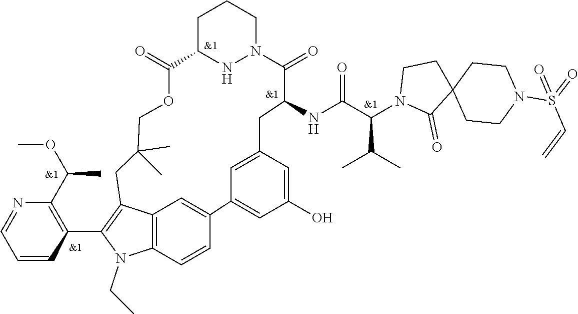 |
| A300 | 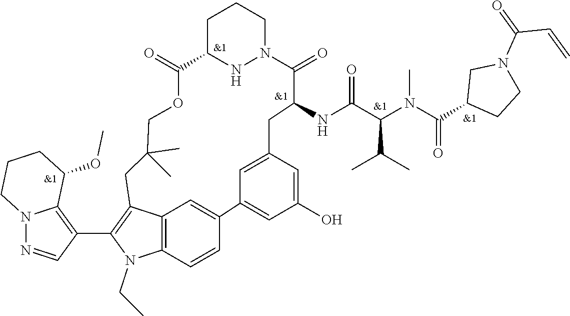 |
| A301 | 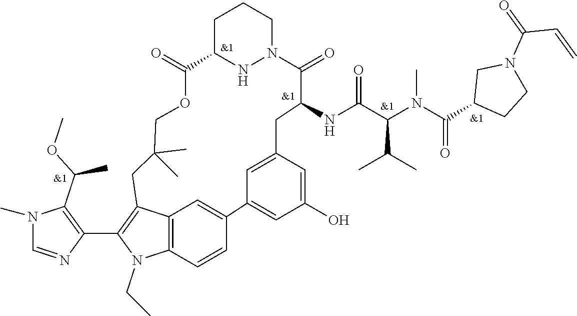 |
| A302 | 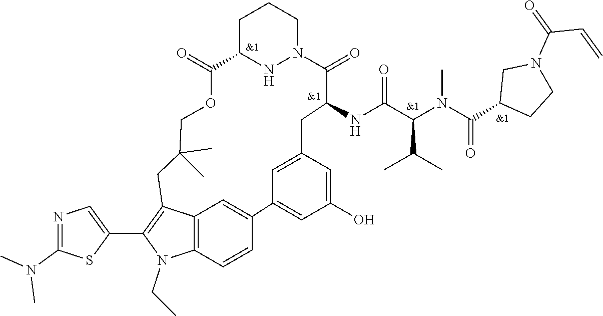 |
| A303 | 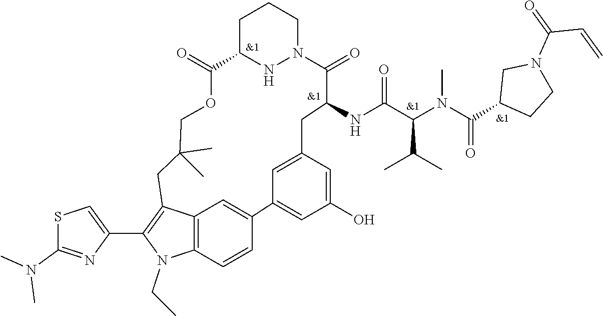 |
| A304 | 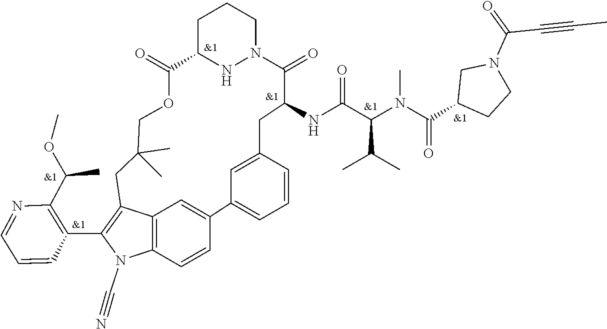 |
| A305 | 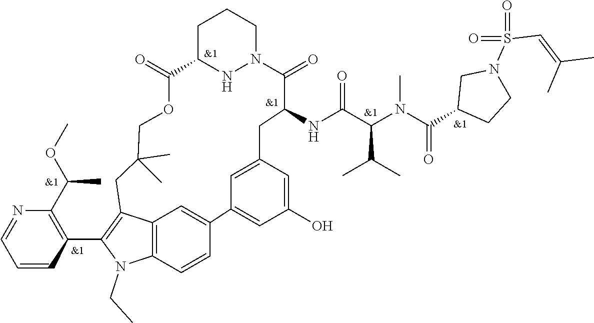 |
| A306 | 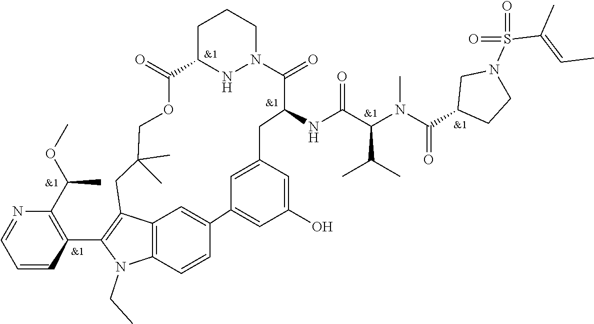 |
| A307 | 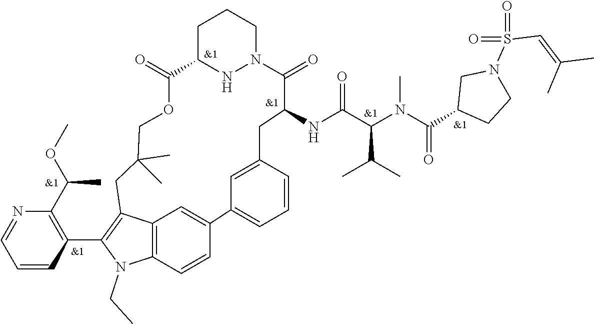 |
| A308 | 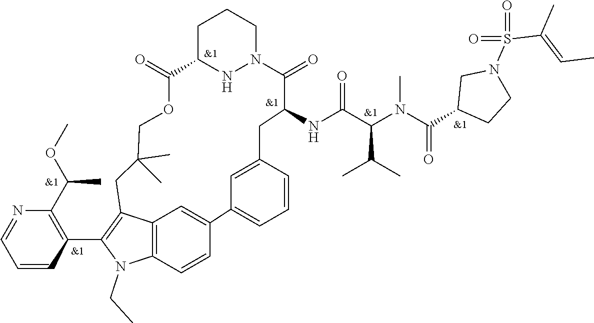 |
| A309 | 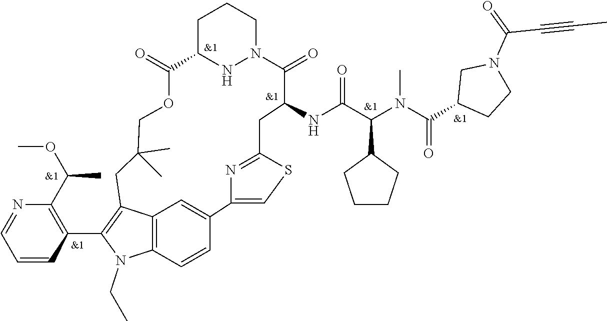 |
| A310 | 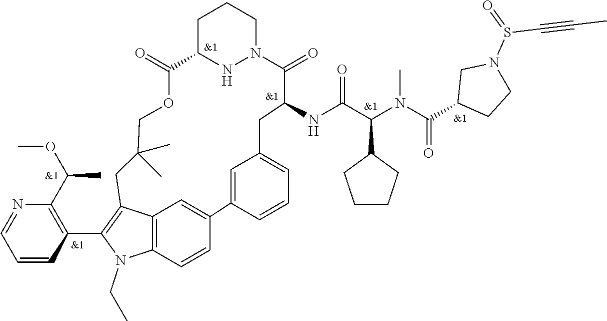 |
| A311 | 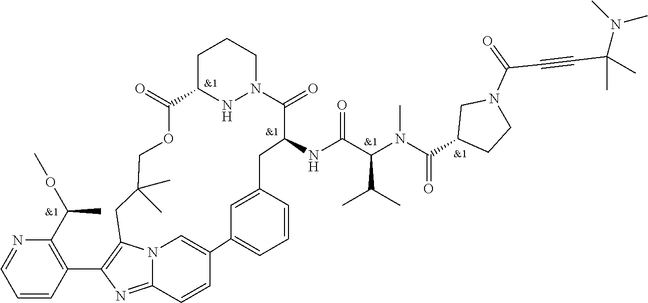 |
| A312 | 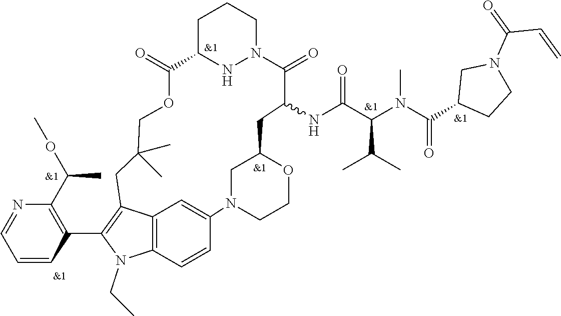 |
| A313 | 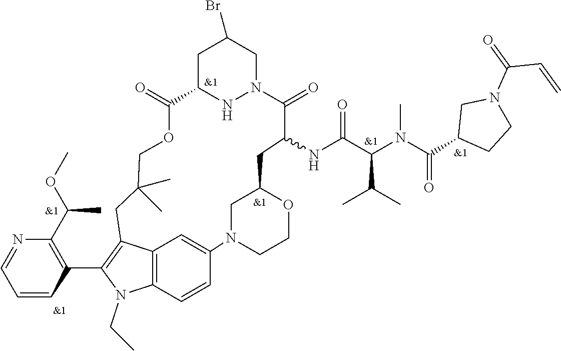 |
| A314 | 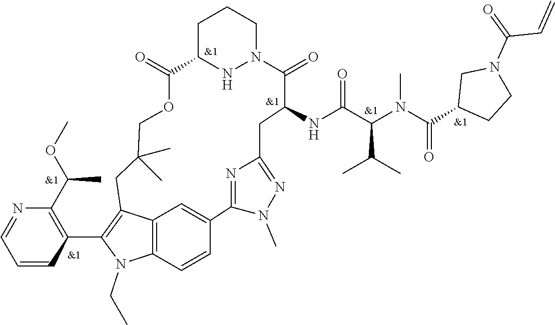 |
| A316 | 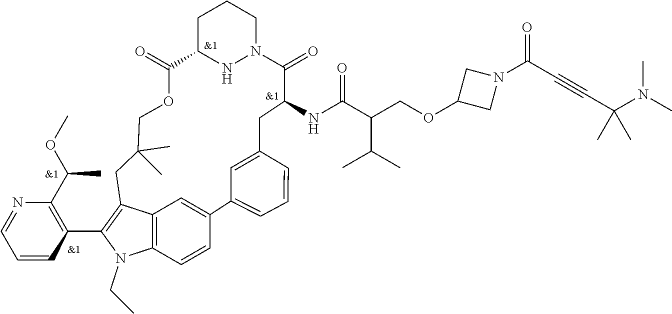 |
| A317 | 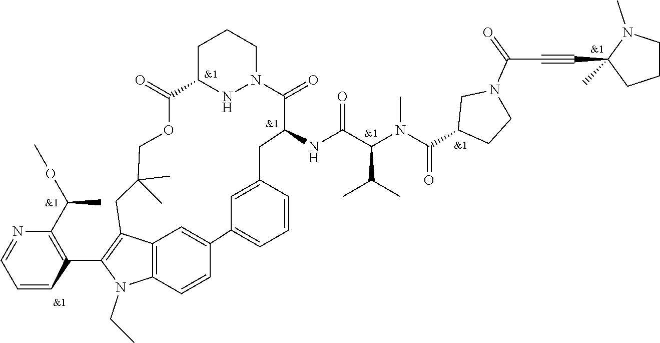 |
| A318 | 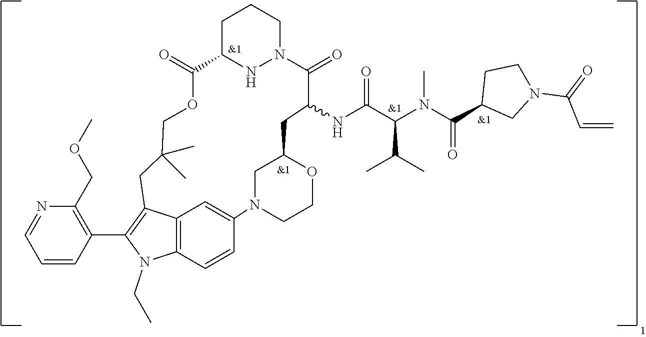 |
| A319 | 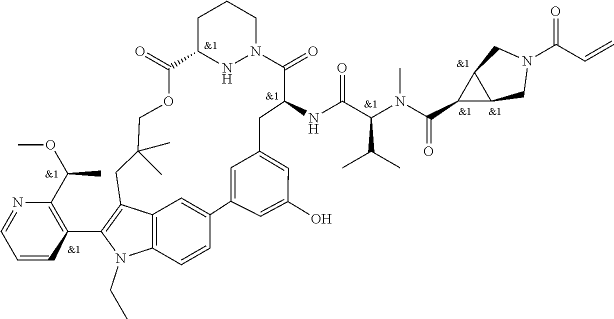 |
| A320 | 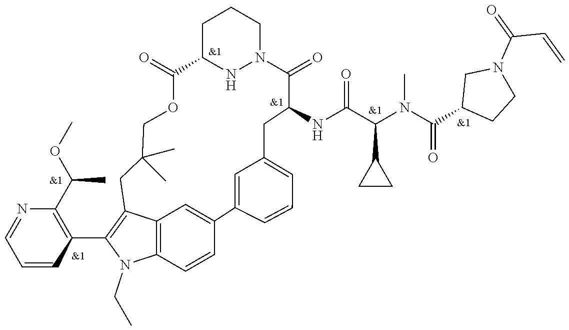 |
| A321 | 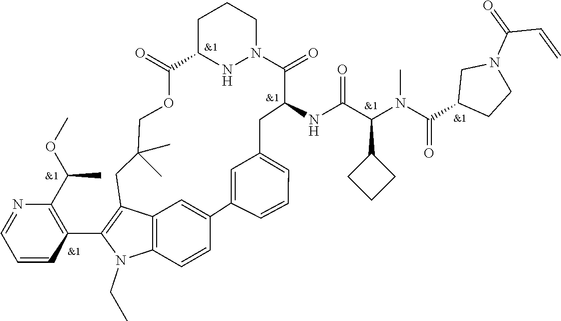 |
| A322 | 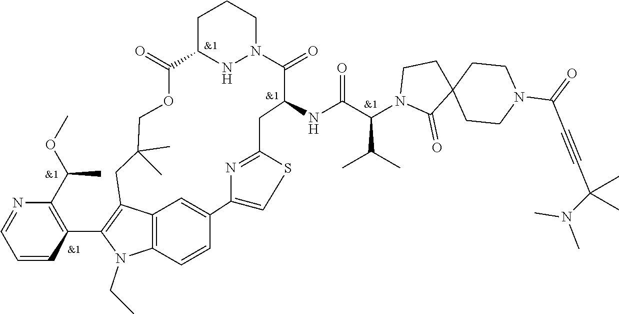 |
| A323 | 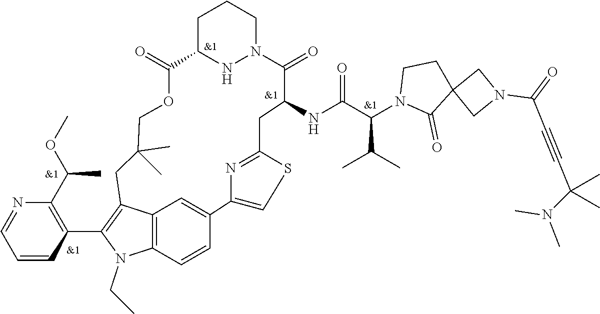 |
| A324 | 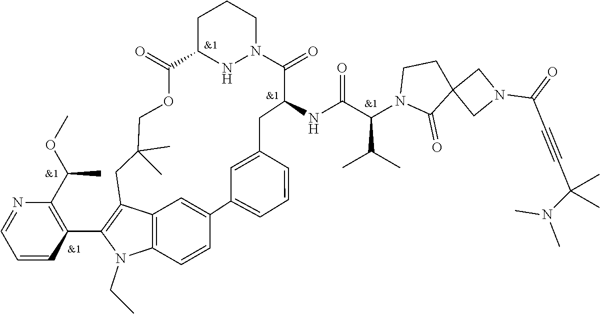 |
| A325 | 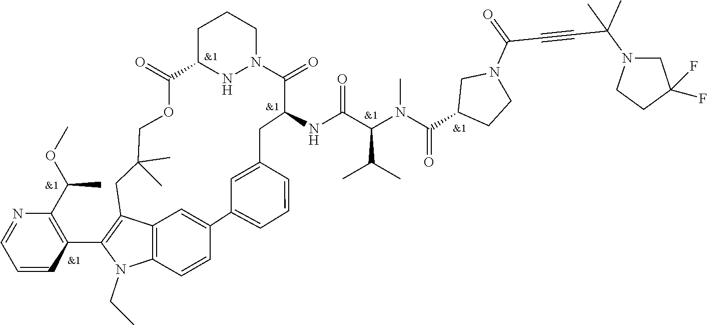 |
| A326 | 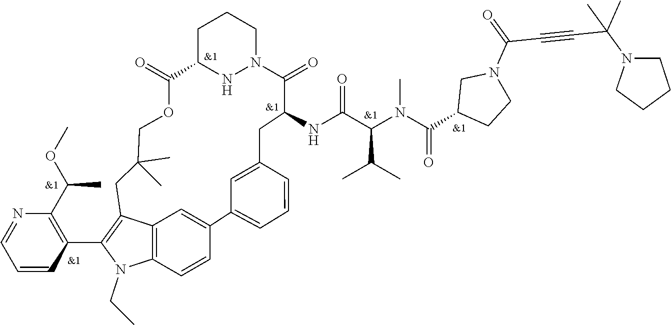 |
| A327 | 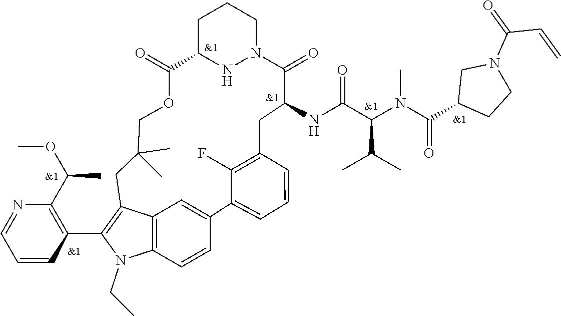 |
| A328 | 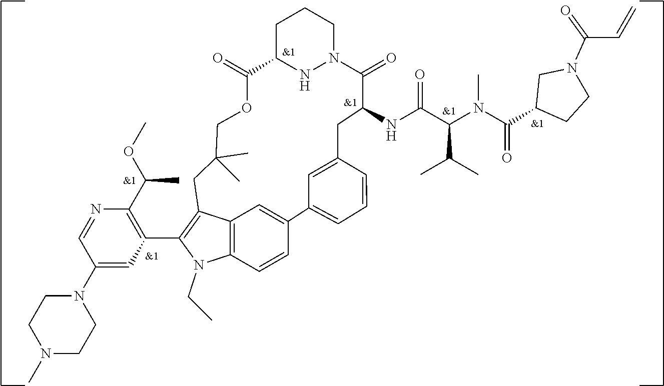 |
| A329 | 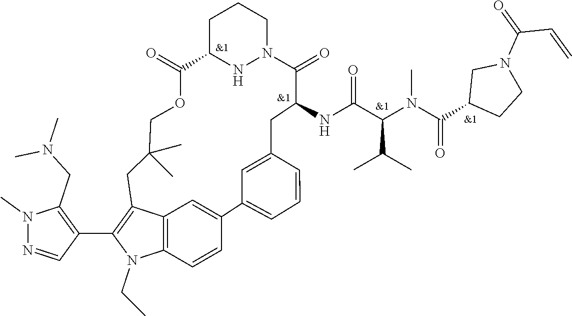 |
| A330 | 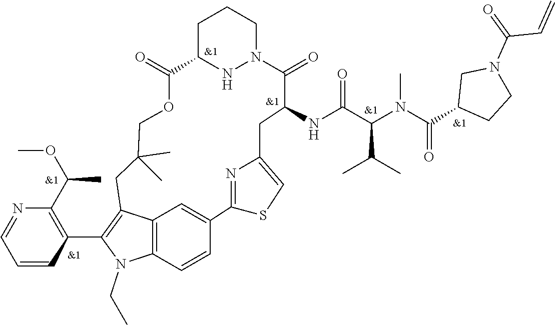 |
| A331 | 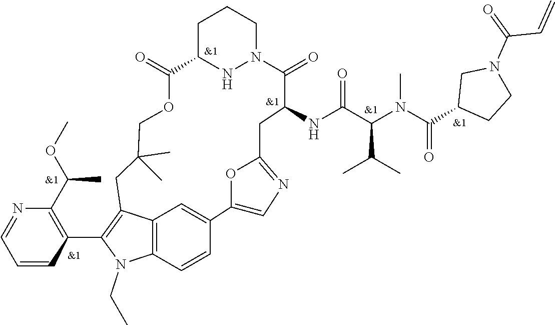 |
| A332 | 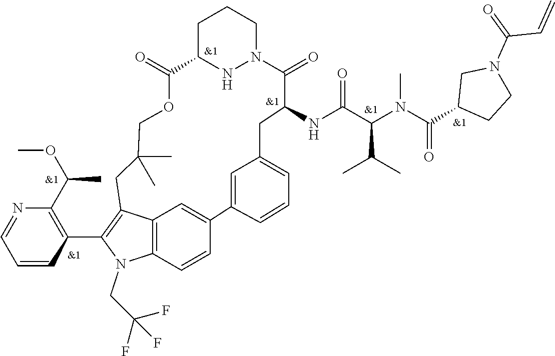 |
| A333 | 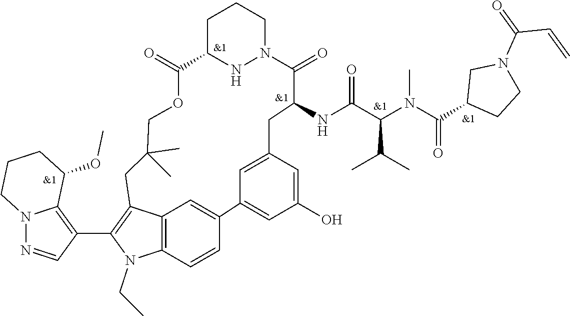 |
| A334 | 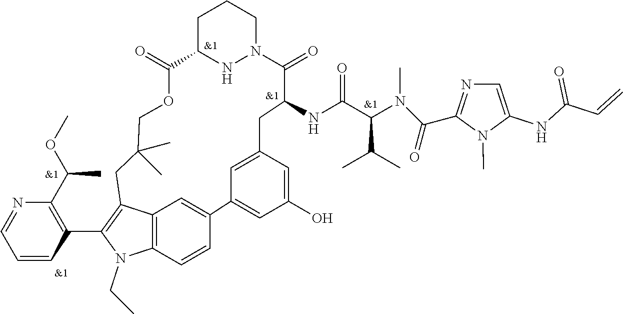 |
| A335 | 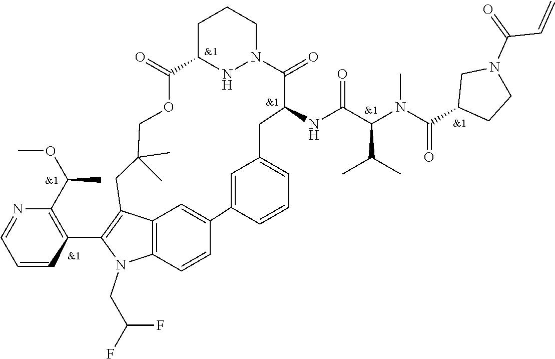 |
| A336 | 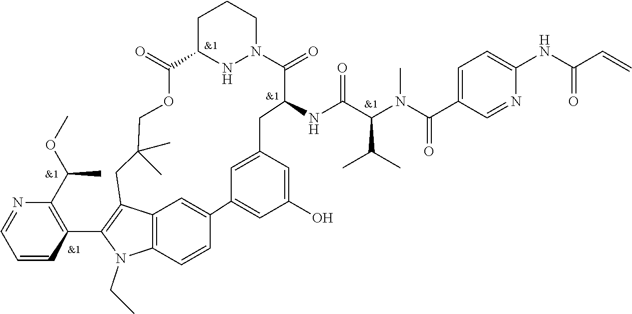 |
| A337 | 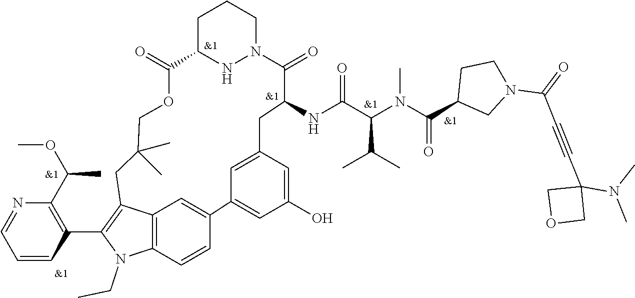 |
| A338 | 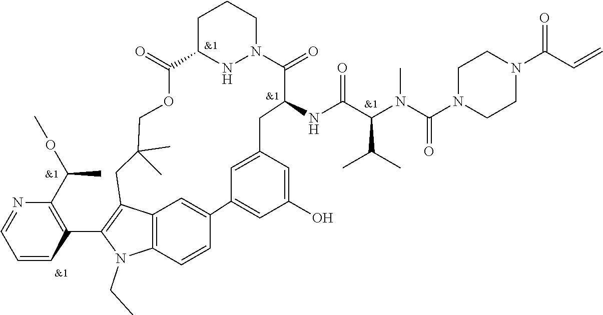 |
| A339 | 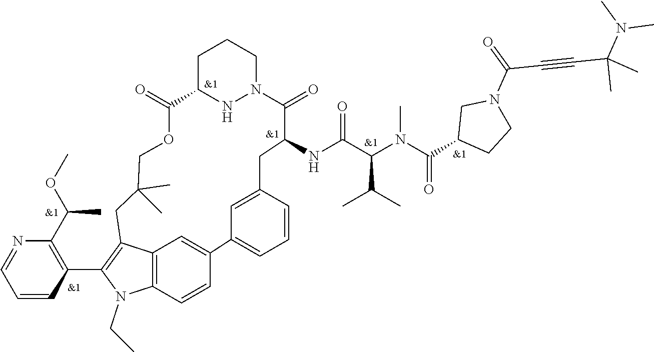 |
| A340 | 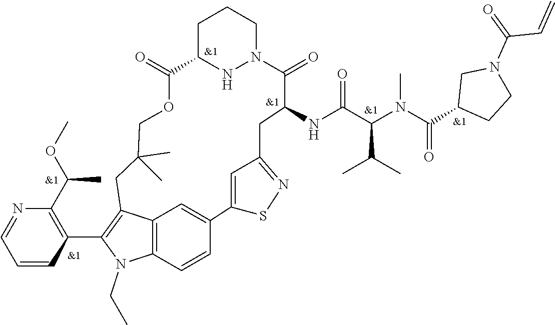 |
| A341 | 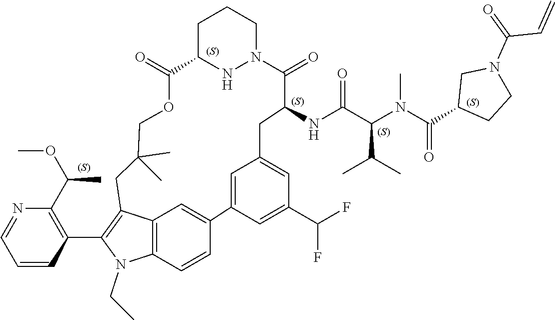 |
| A342 | 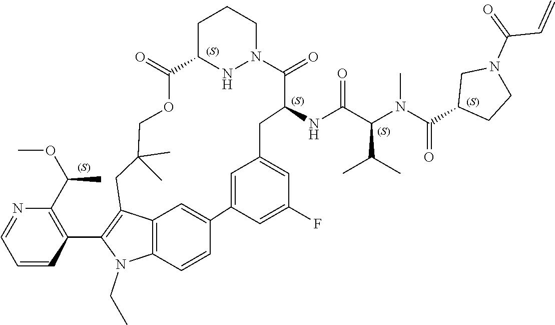 |
| A343 | 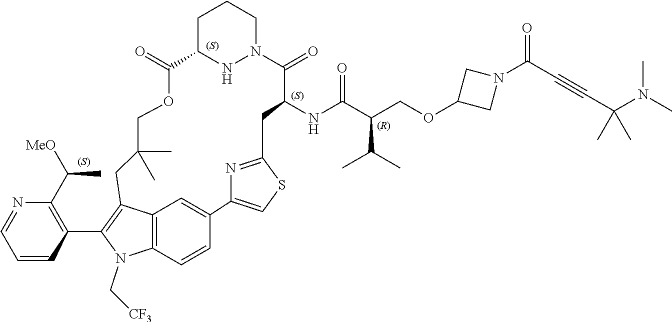 |
| A344 | 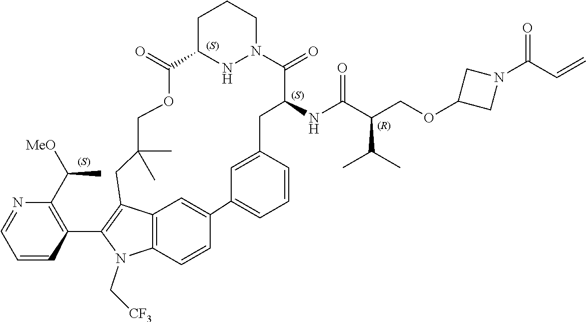 |
| A345 | 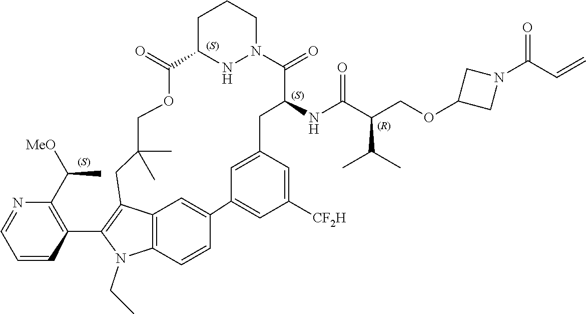 |
| A346 | 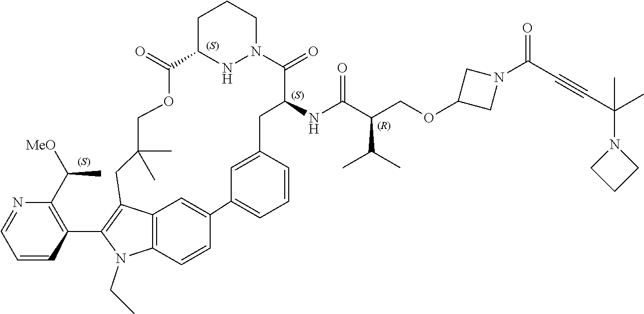 |
| A347 | 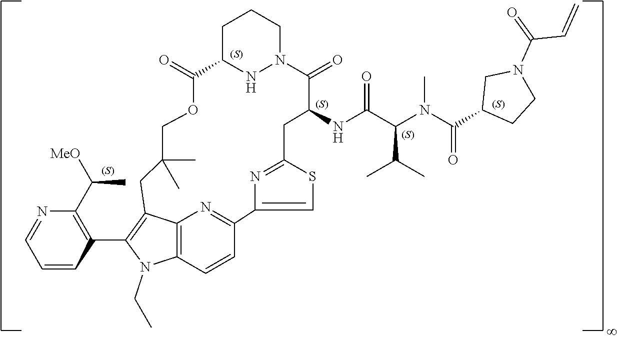 |
| A348 | 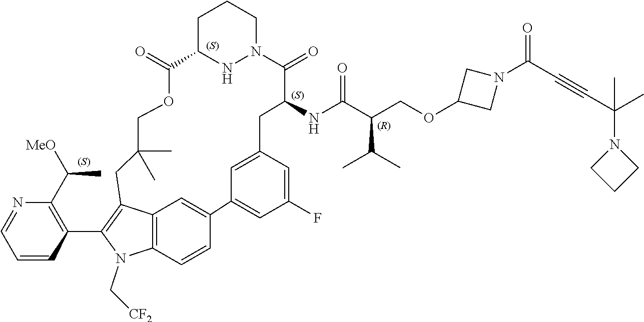 |
| A349 | 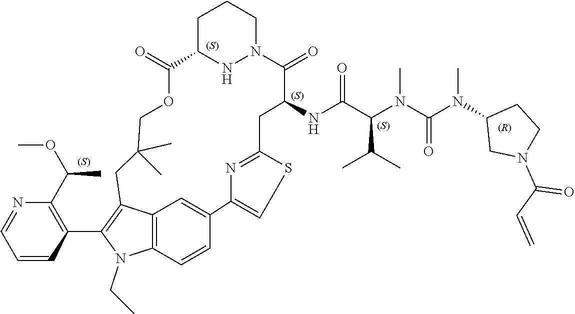 |
| A350 | 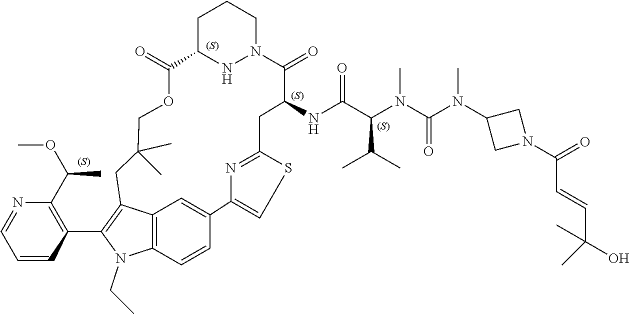 |
| A351 | 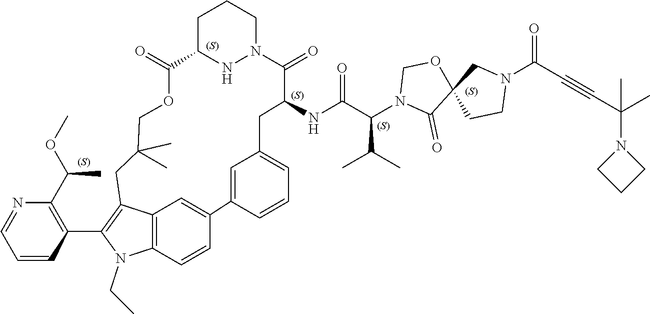 |
| A352 | 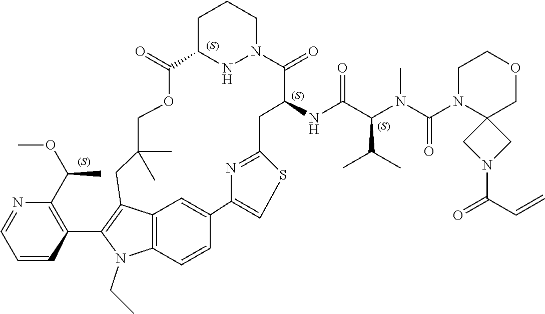 |
| A353 | 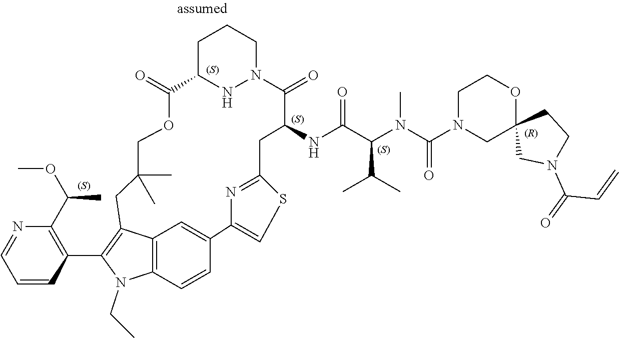 |
| A354 | 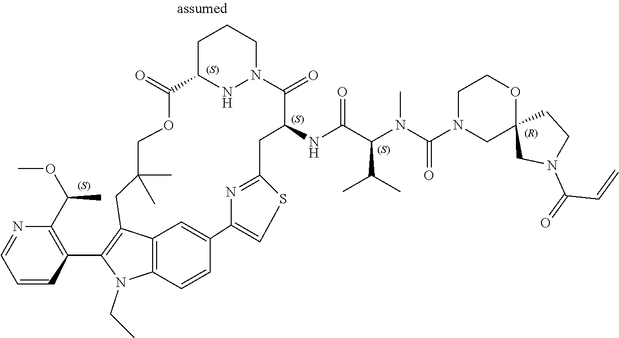 |
| A355 | 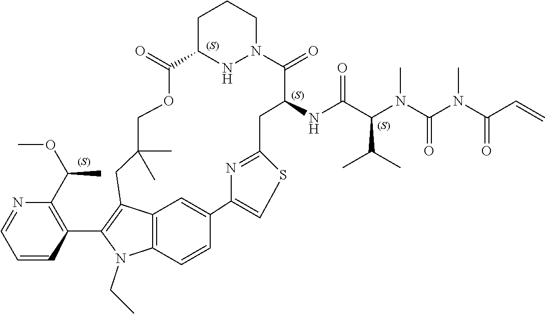 |
| A356 | 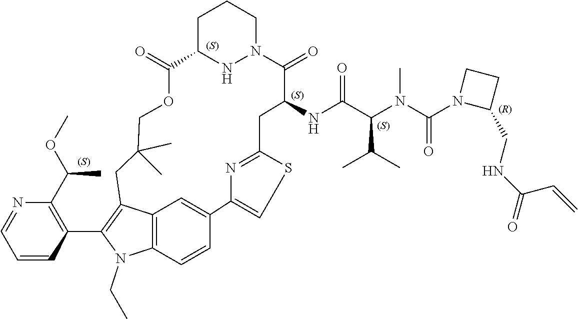 |
| A357 | 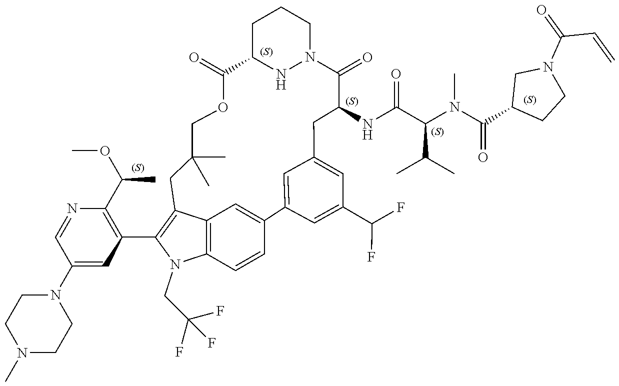 |
| A358 | 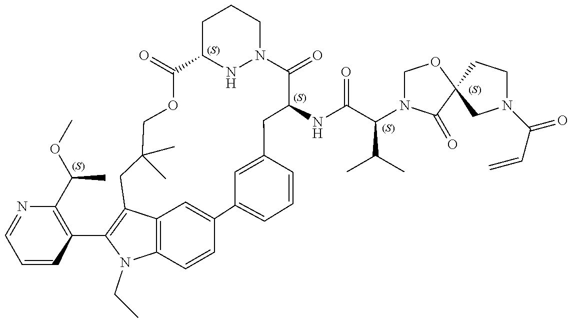 |
| A359 | 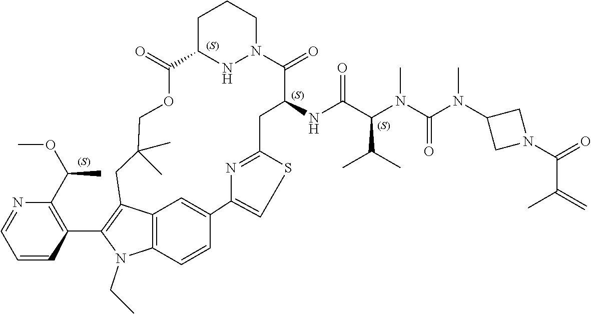 |
| A360 | 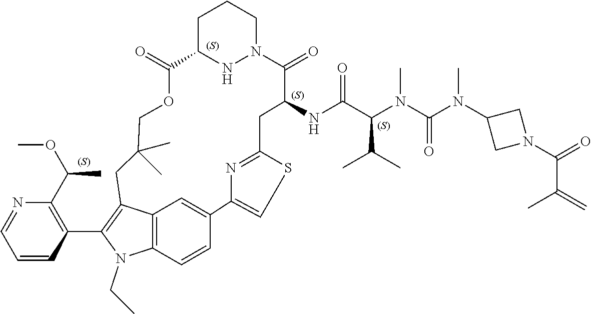 |
| A361 | 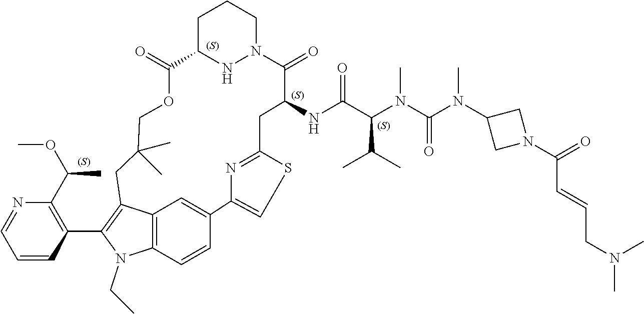 |
| A362 | 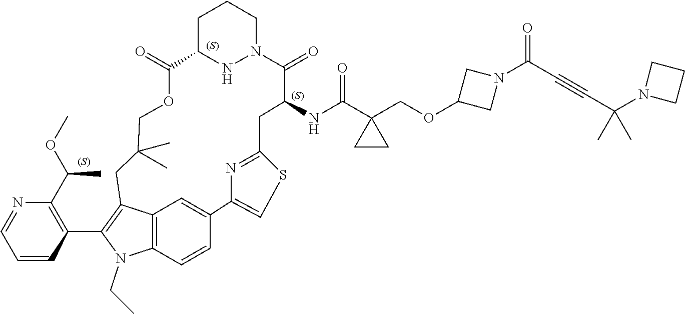 |
| A363 | 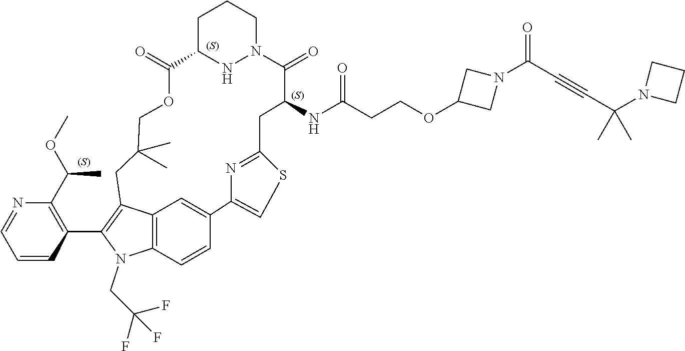 |
| A364 | 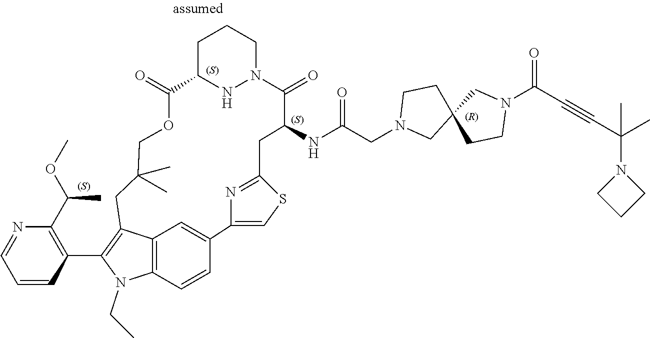 |
| A365 | 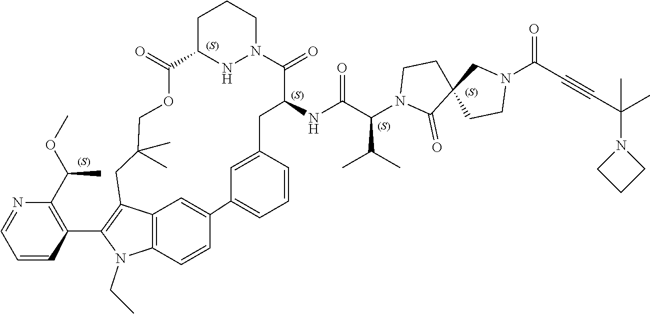 |
| A366 | 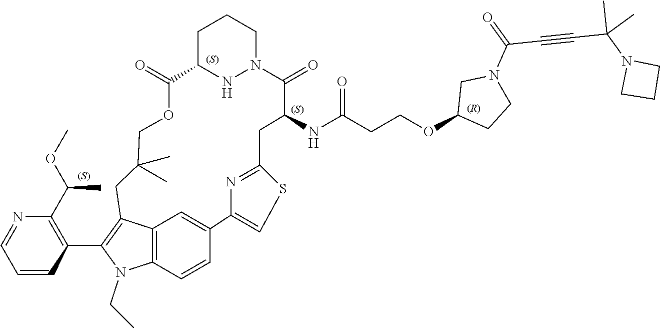 |
| A367 | 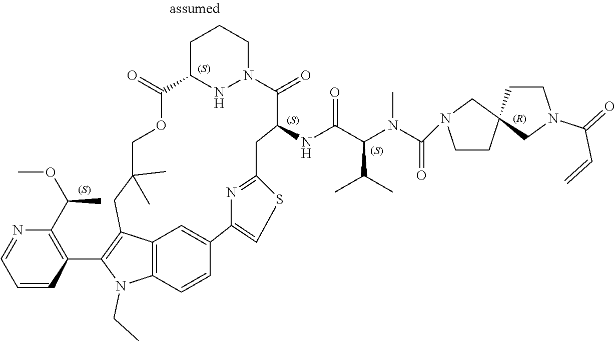 |
| A368 | 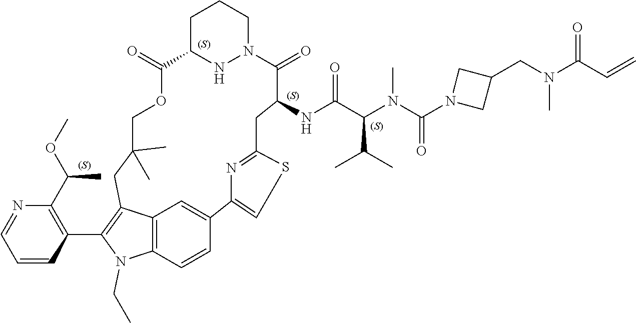 |
| A369 | 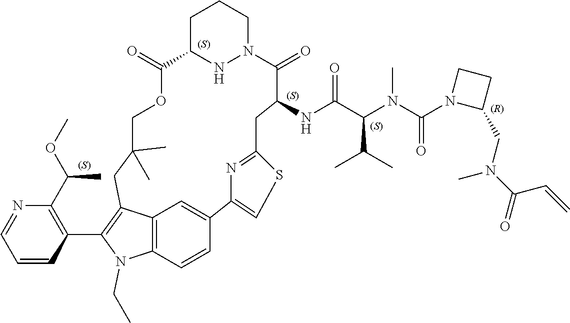 |
| A370 | 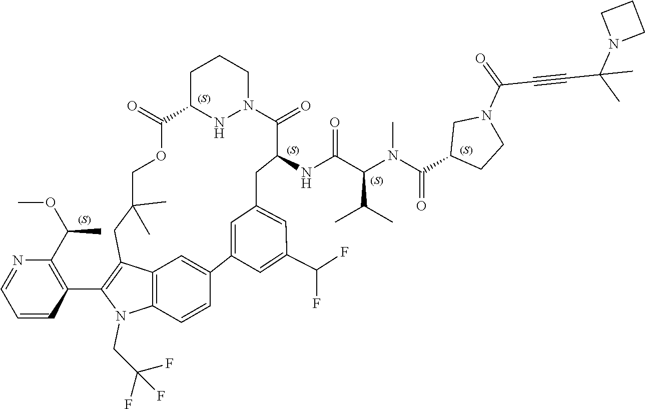 |
| A371 | 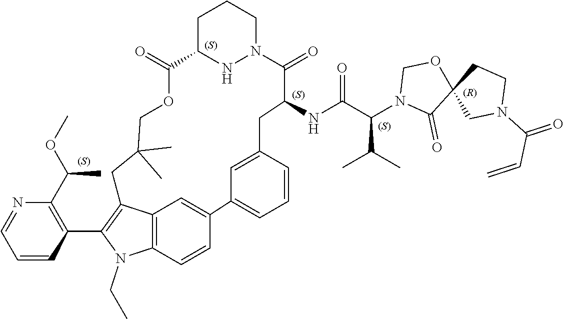 |
| A372 | 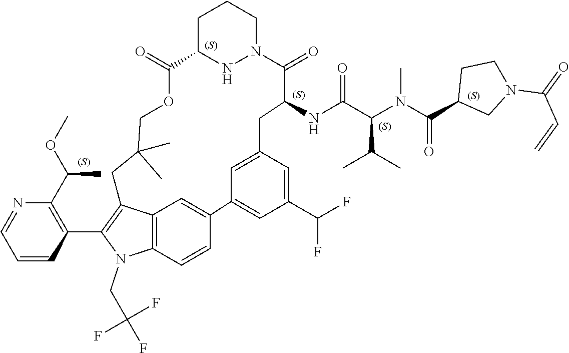 |
| A373 | 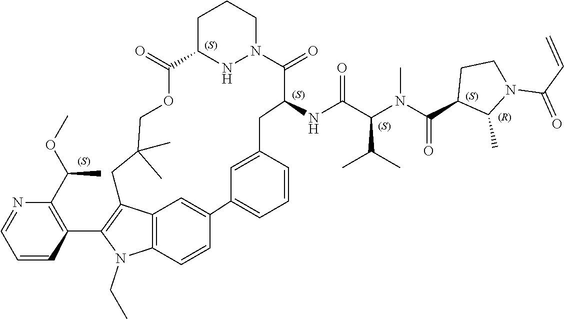 |
| A374 | 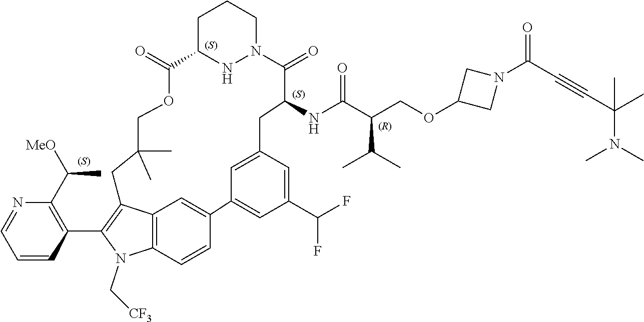 |
| A375 | 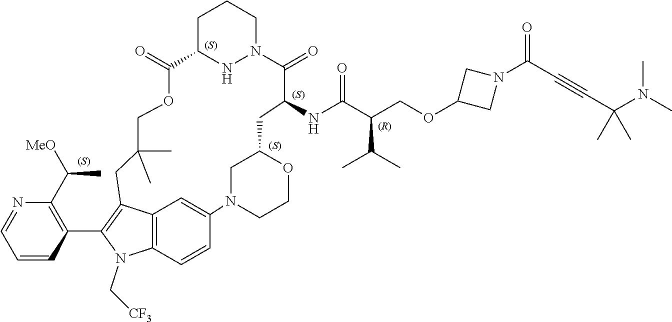 |
| A376 | 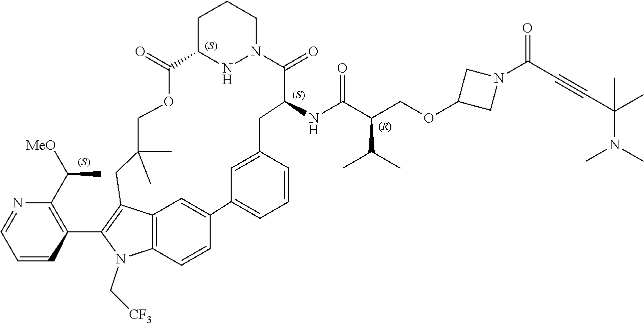 |
| A377 | 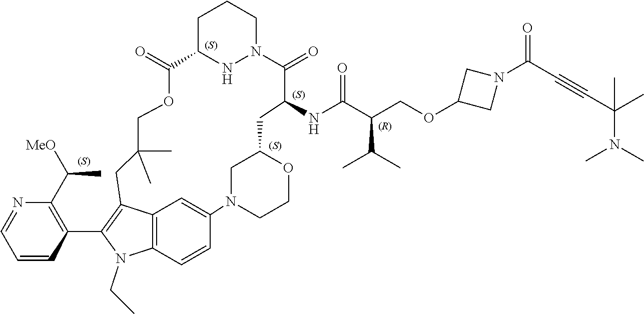 |
| A378 | 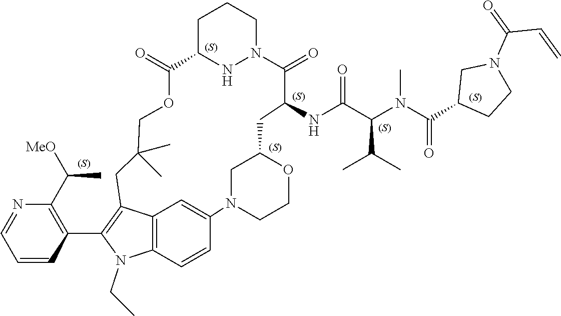 |
| A379 | 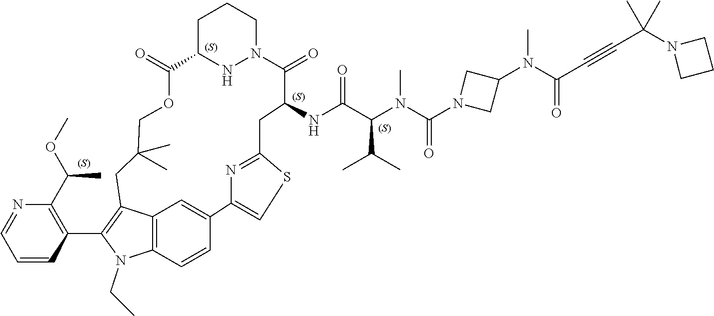 |
| A380 | 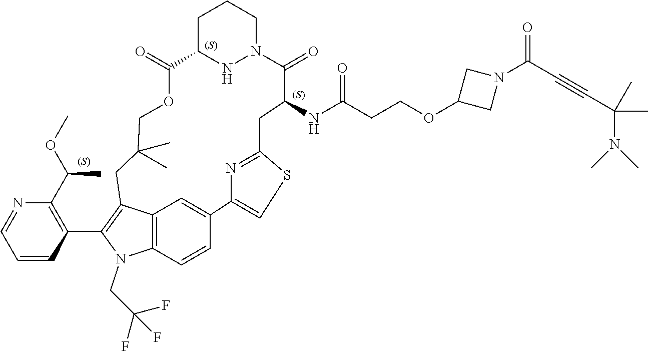 |
| A381 | 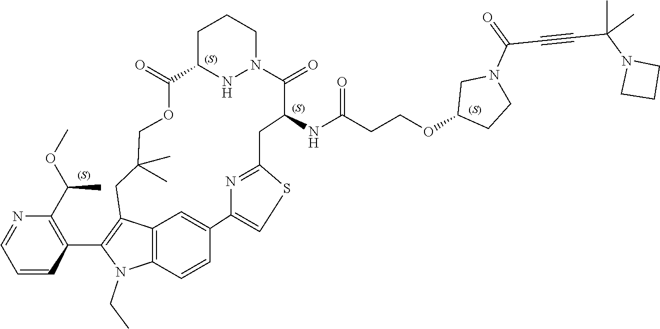 |
| A382 | 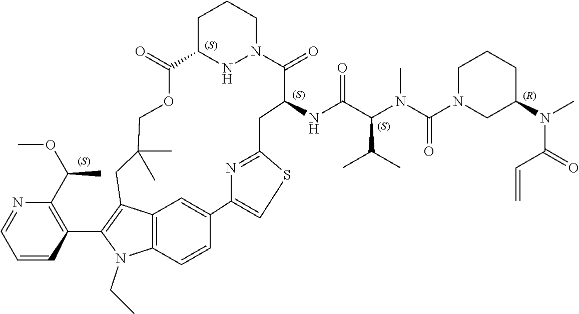 |
| A383 | 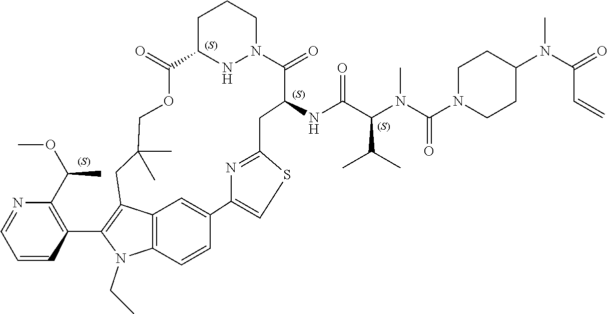 |
| A384 | 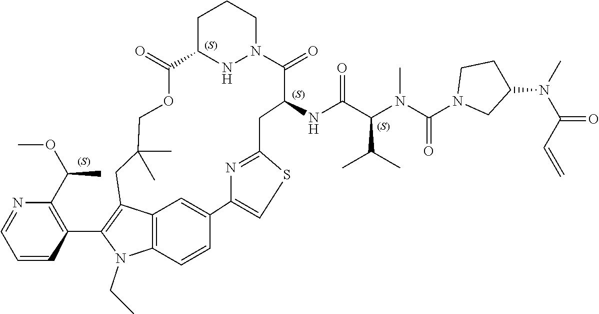 |
| A385 | 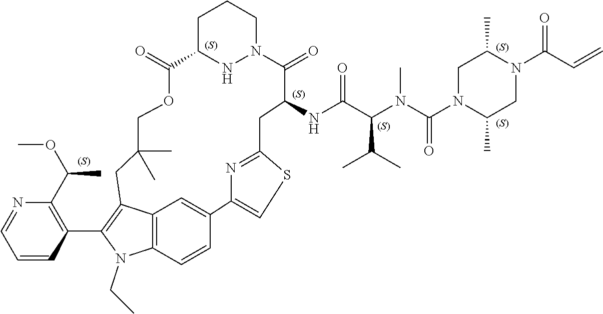 |
| A386 | 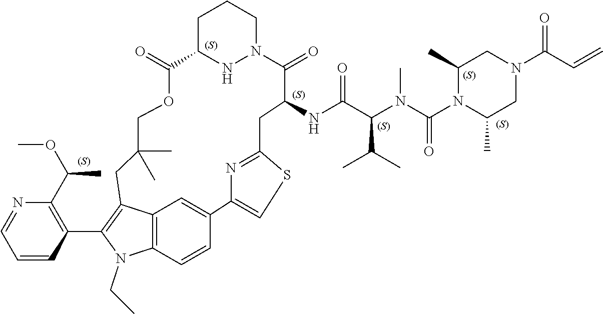 |
| A387 | 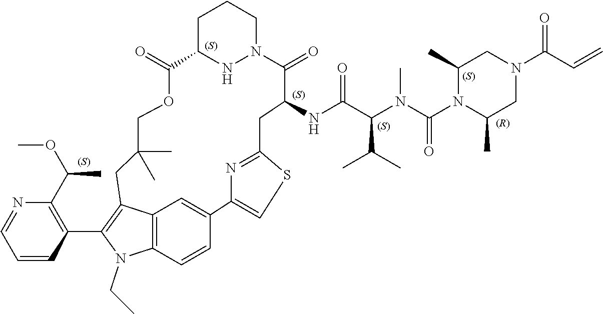 |
| A388 | 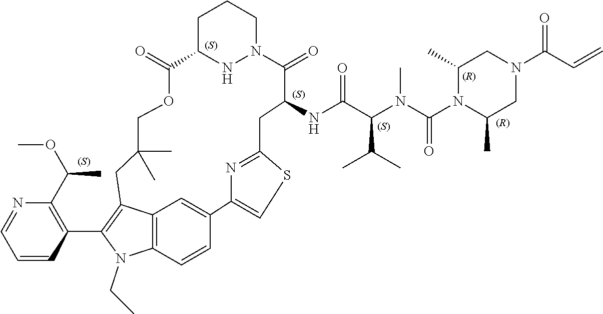 |
| A389 | 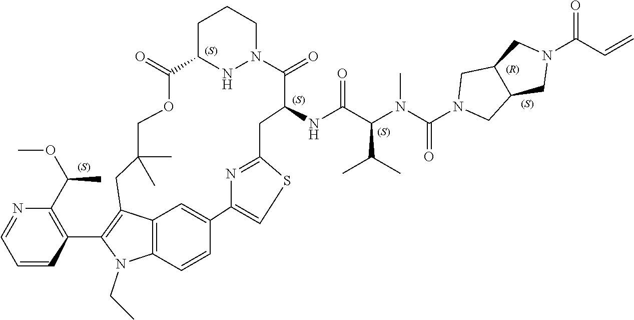 |
| A390 | 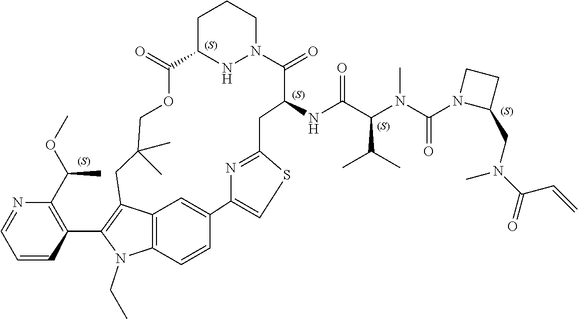 |
| A391 | 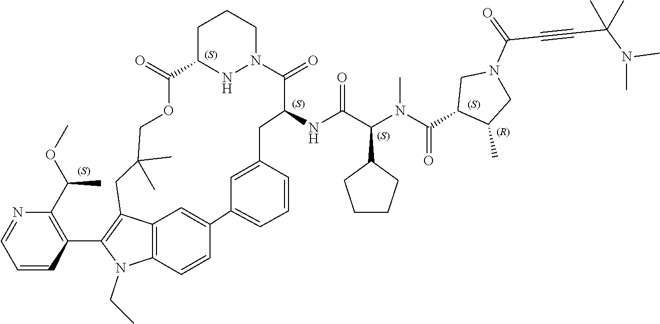 |
| A392 | 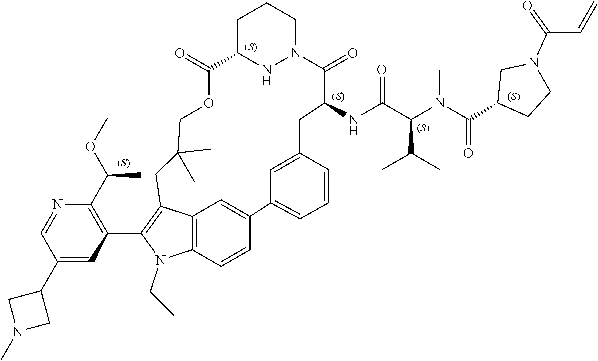 |
| A393 | 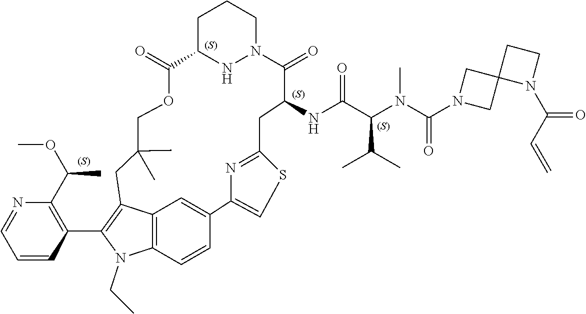 |
| A394 | 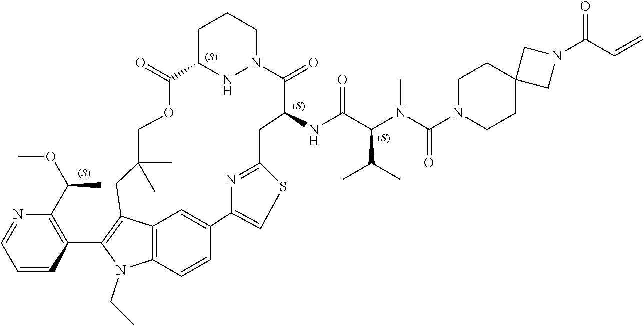 |
| A395 | 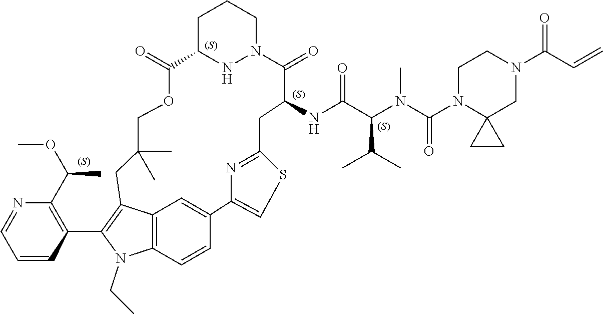 |
| A396 | 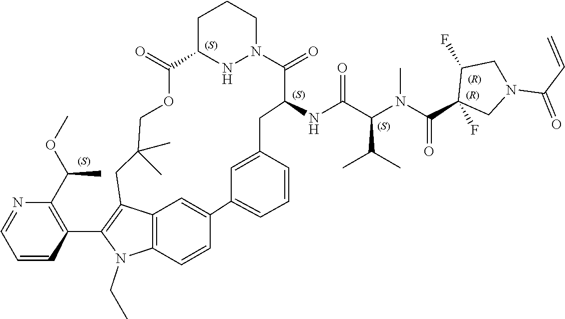 |
| A397 | 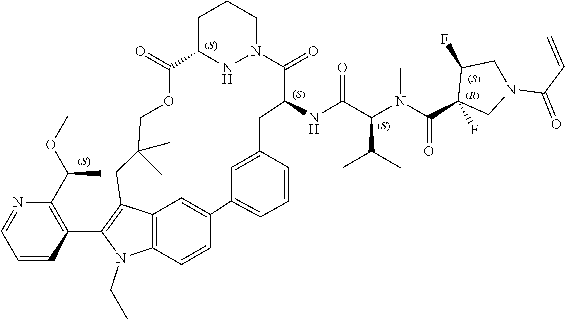 |
| A398 | 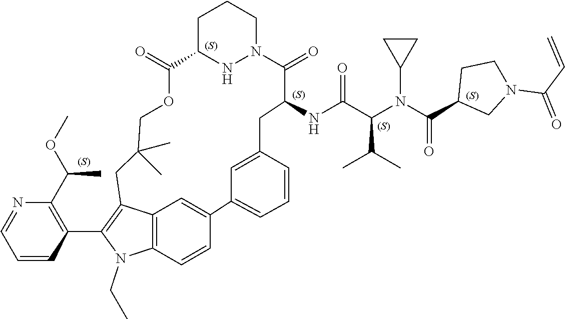 |
| A399 | 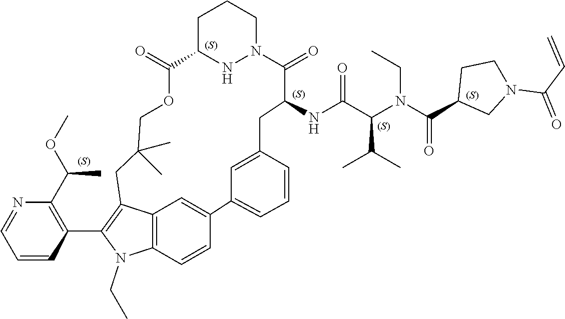 |
| A400 | 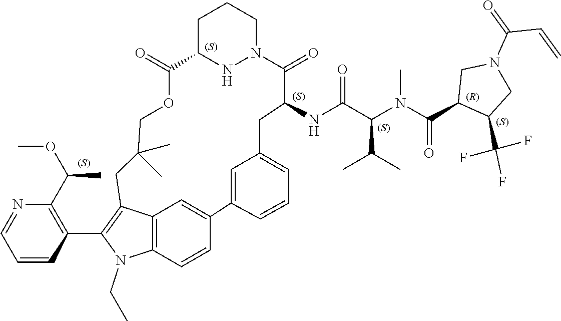 |
| A401 | 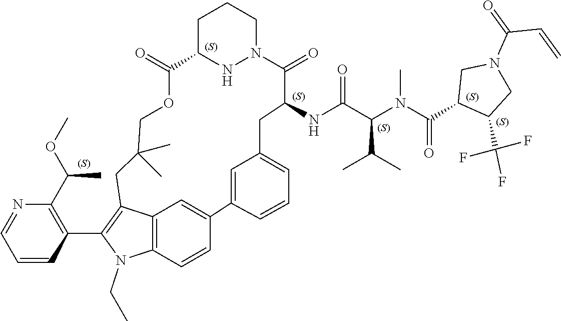 |
| A402 | 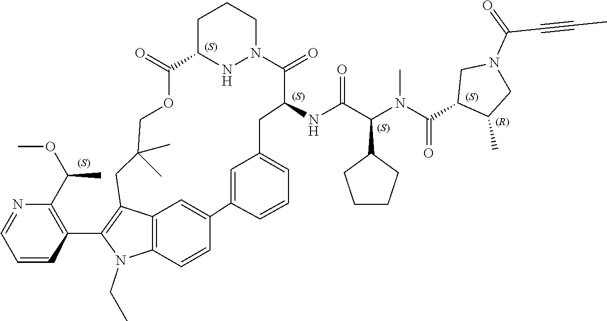 |
| A403 | 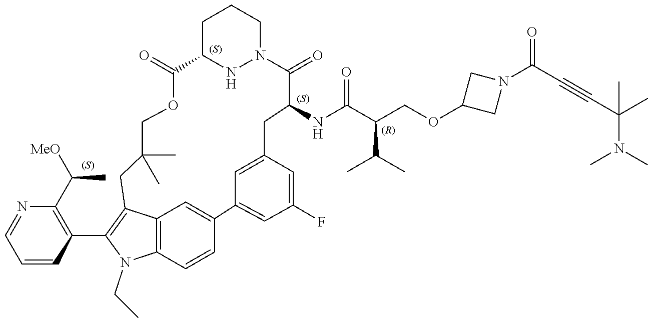 |
| A404 | 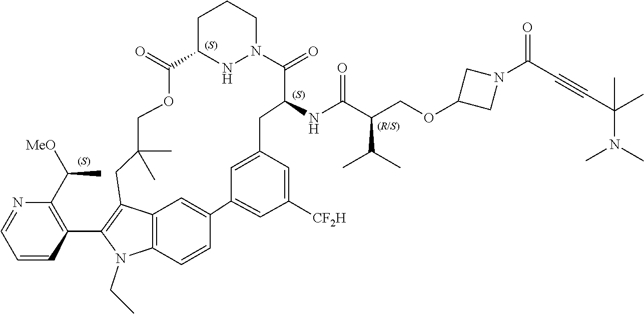 |
| A405 | 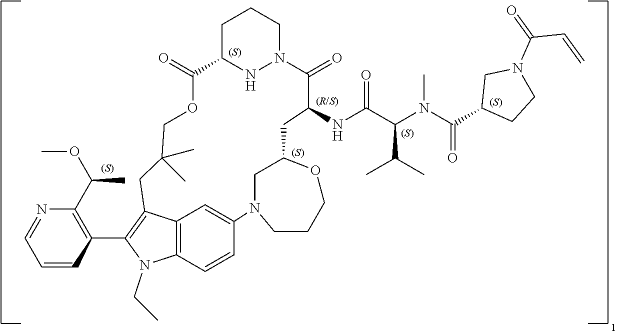 |
| A406 | 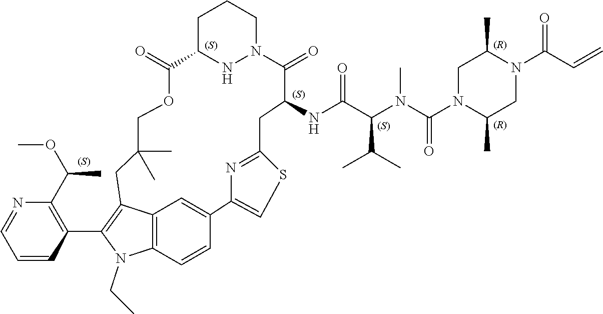 |
| A407 | 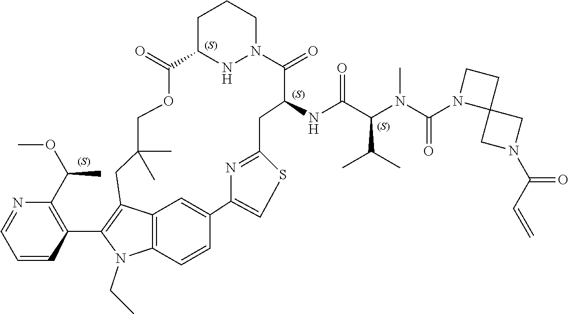 |
| A408 | 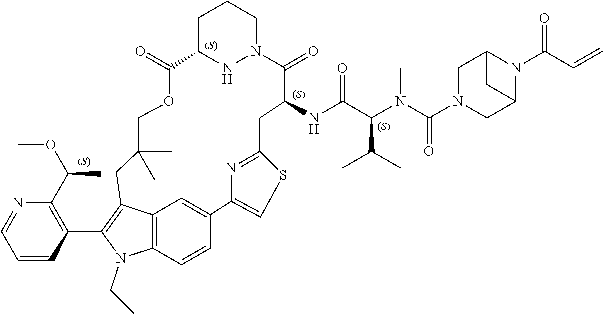 |
| A409 | 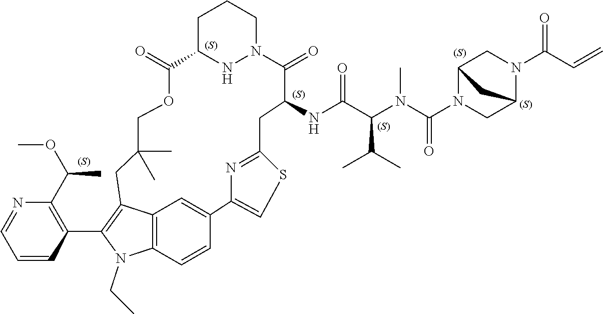 |
| A410 | 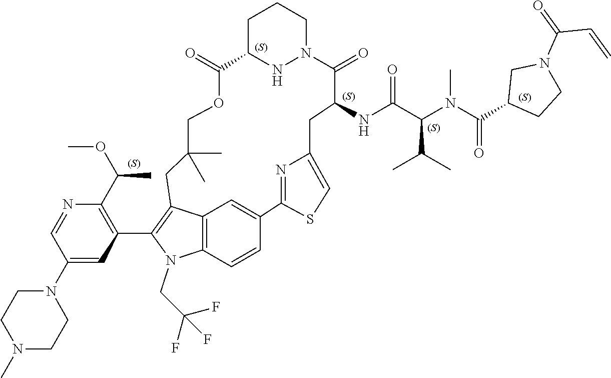 |
| A411 | 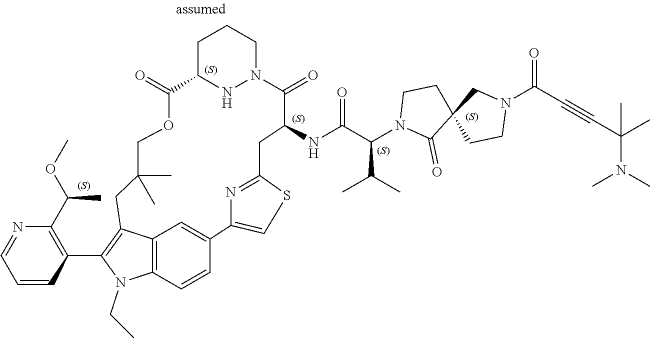 |
| A412 | 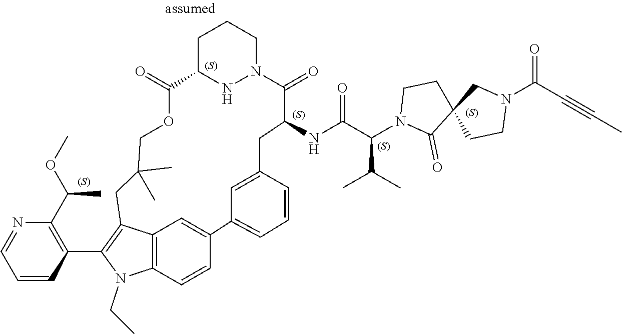 |
| A413 | 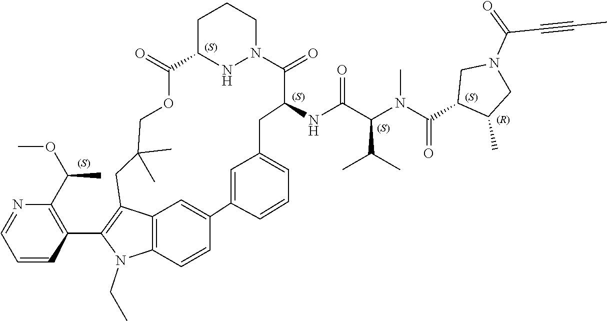 |
| A414 | 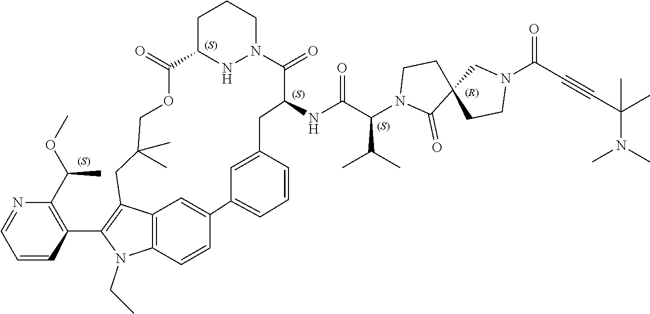 |
| A415 | 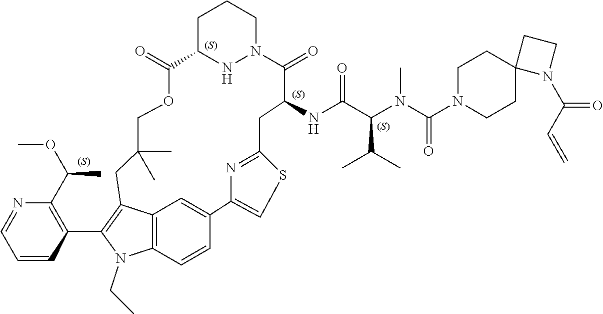 |
| A416 | 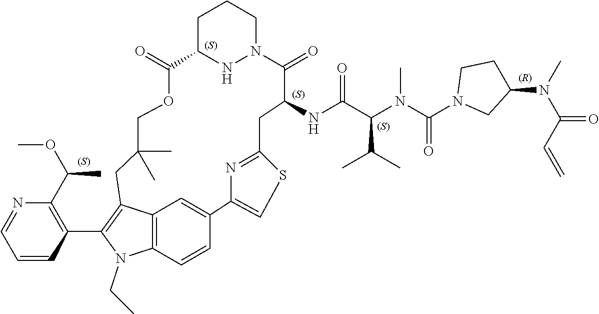 |
| A417 | 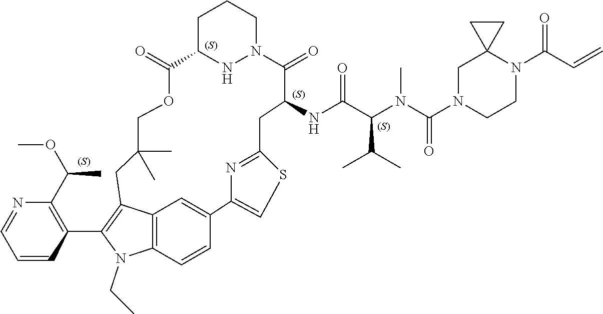 |
| A418 | 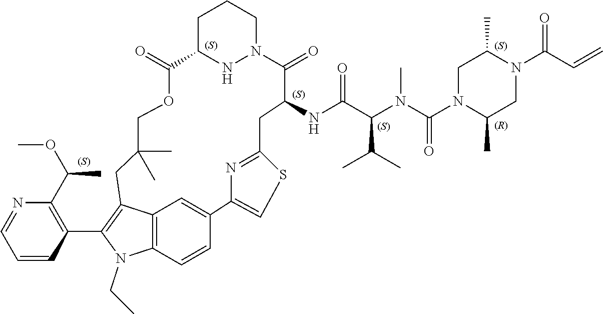 |
| A419 | 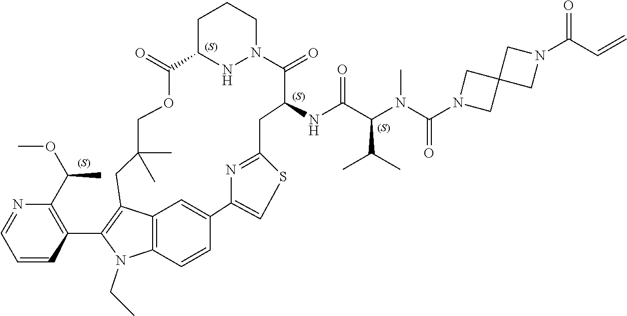 |
| A420 | 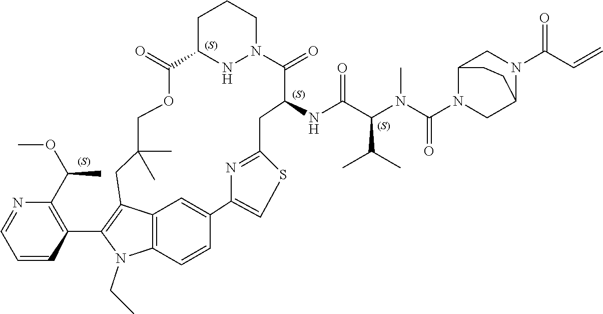 |
| A421 | 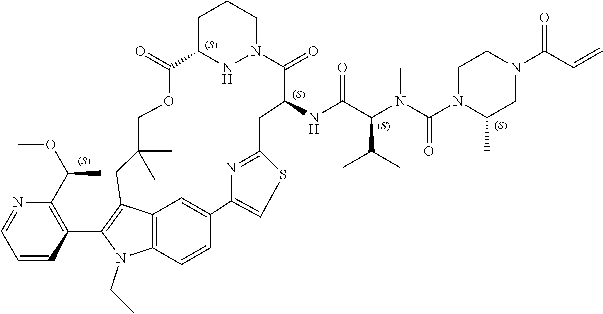 |
| A422 | 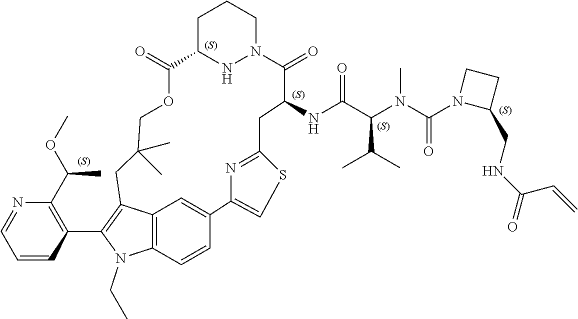 |
| A423 | 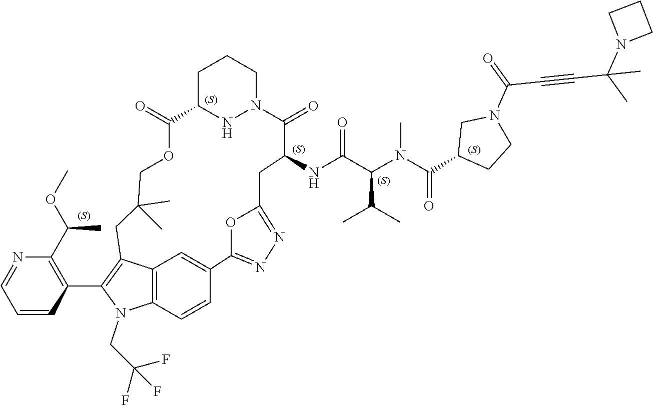 |
| A424 | 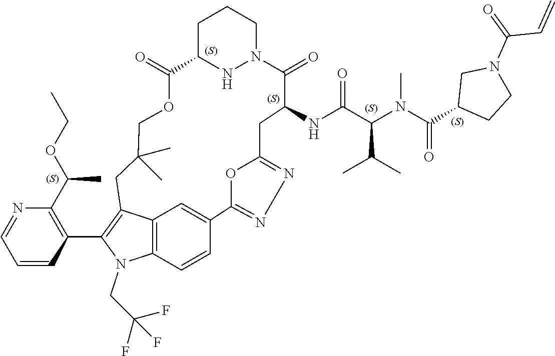 |
| A425 | 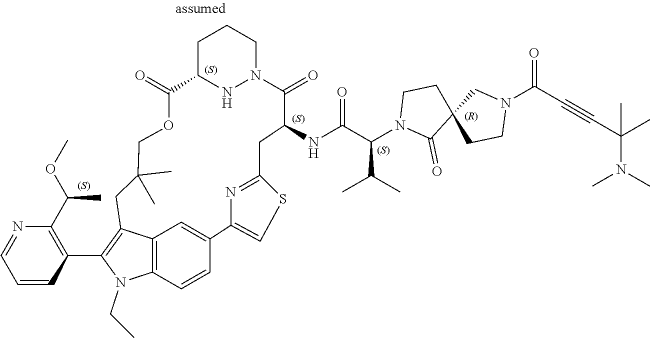 |
| A426 | 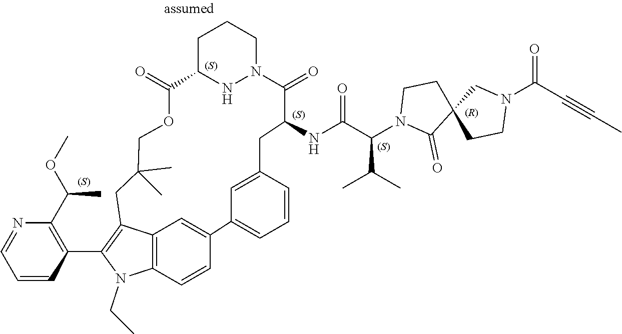 |
| A427 | 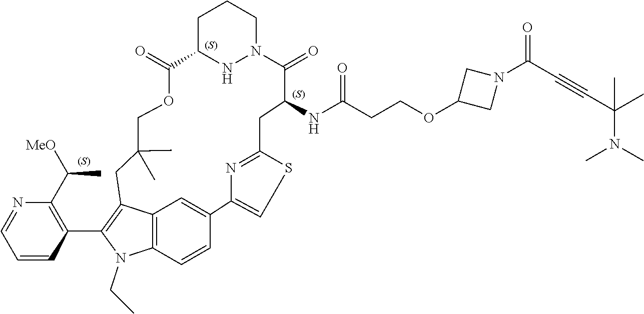 |
| A428 | 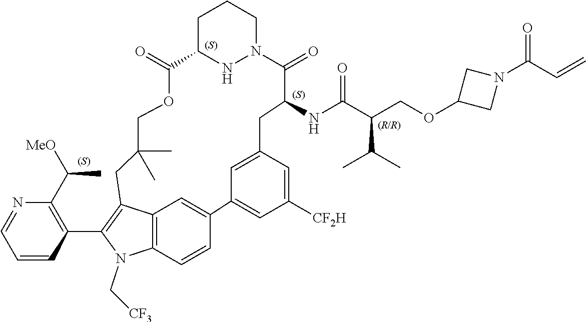 |
| A429 | 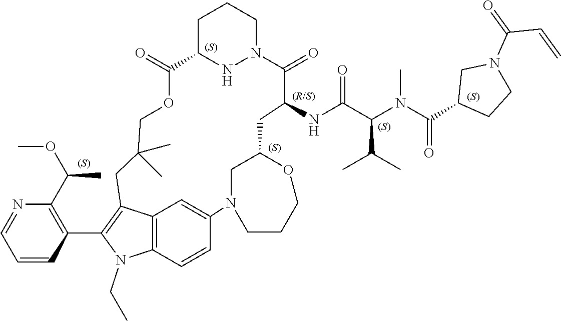 |
| A430 | 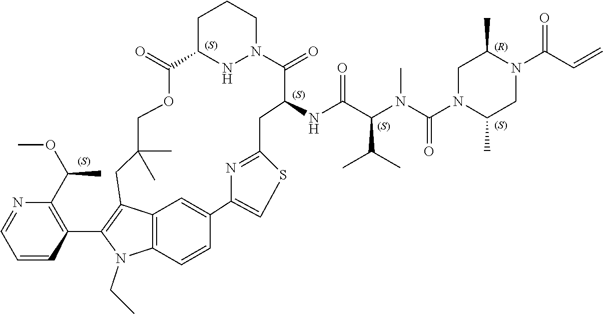 |
| A431 | 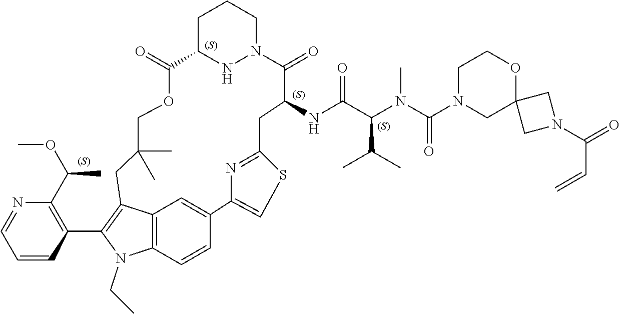 |
| A432 | 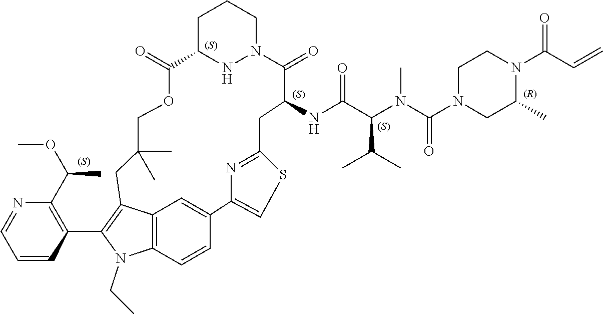 |
| A433 | 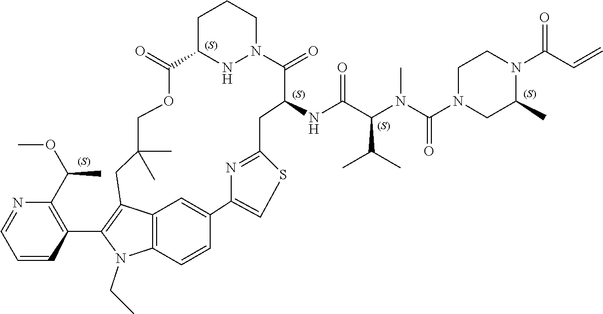 |
| A334 | 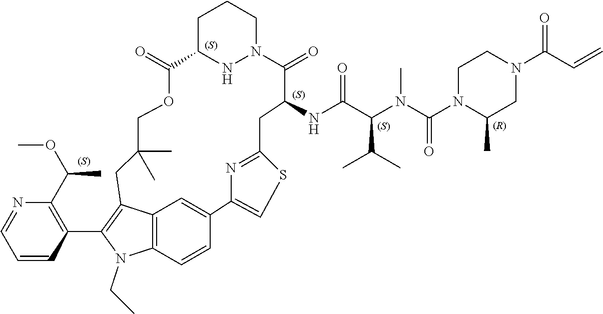 |
| A435 | 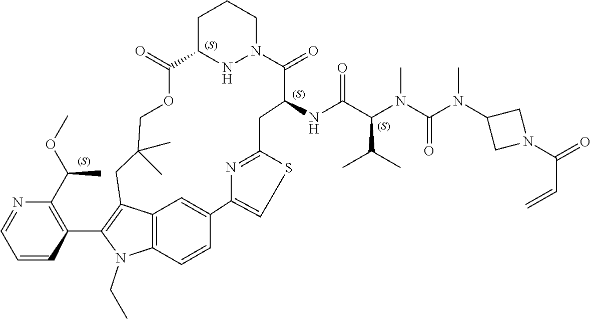 |
| A436 | 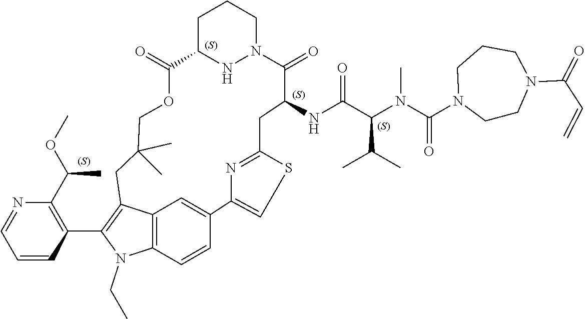 |
| A437 | 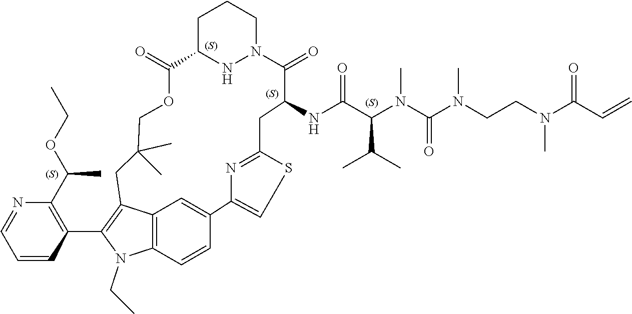 |
| A438 | 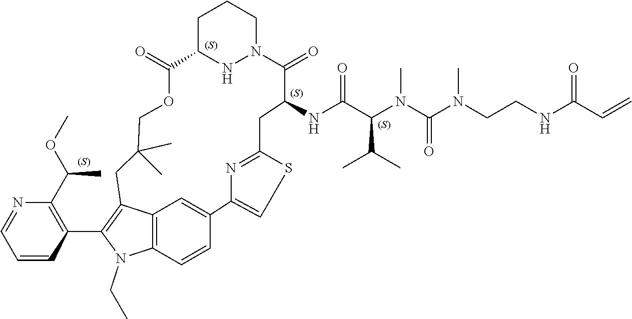 |
| A439 | 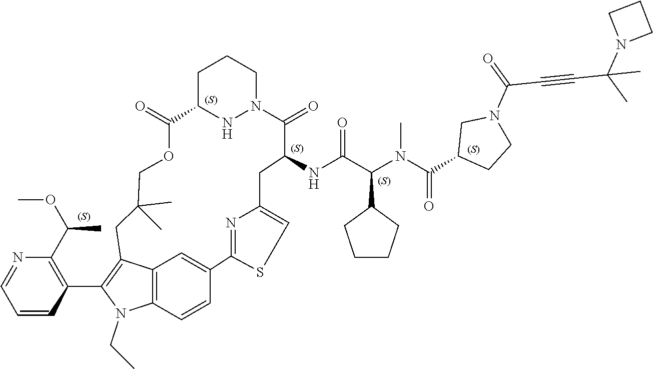 |
| A440 | 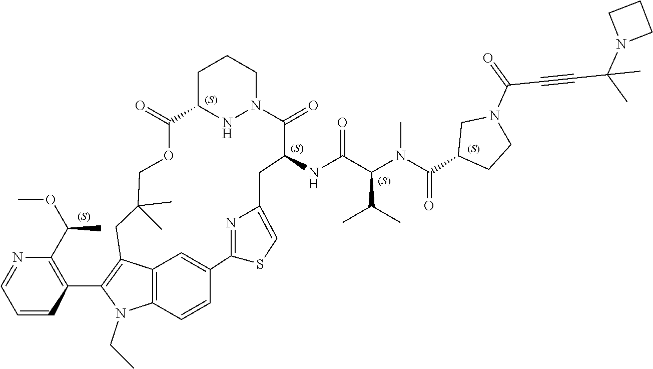 |
| A441 | 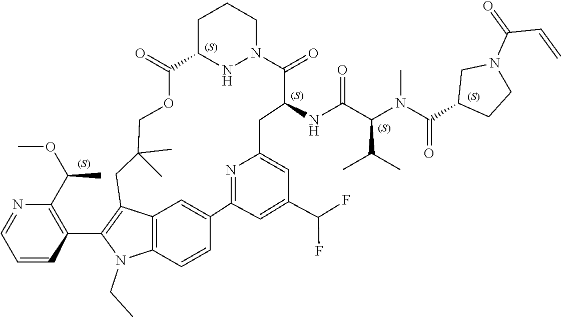 |
| A442 | 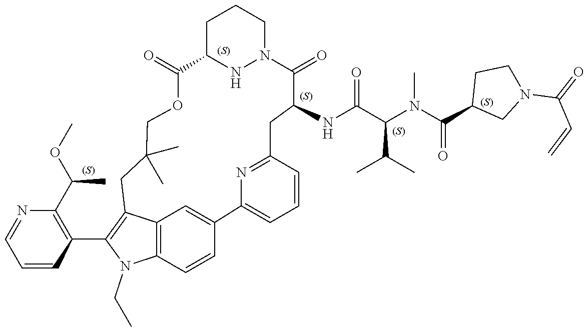 |
| A443 | 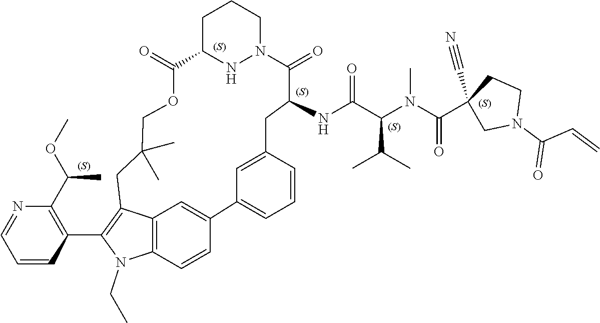 |
| A444 | 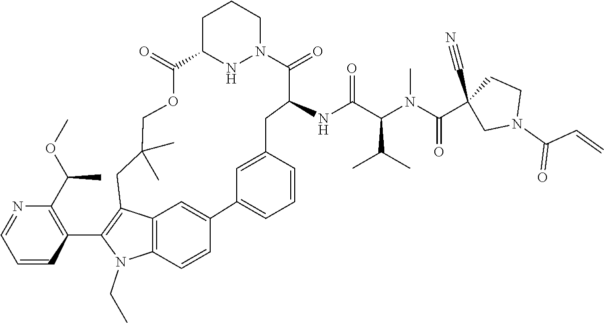 |
| A445 | 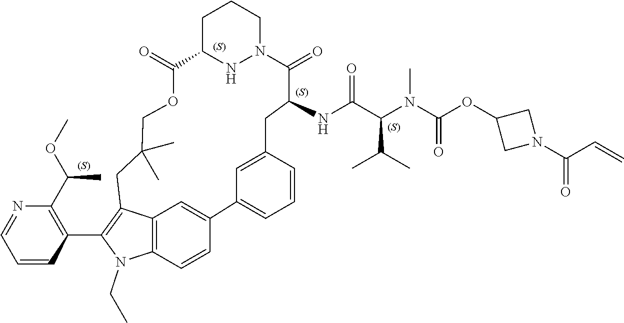 |
| A446 | 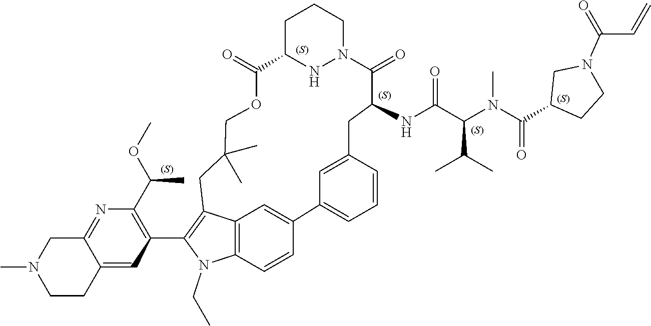 |
| A447 | 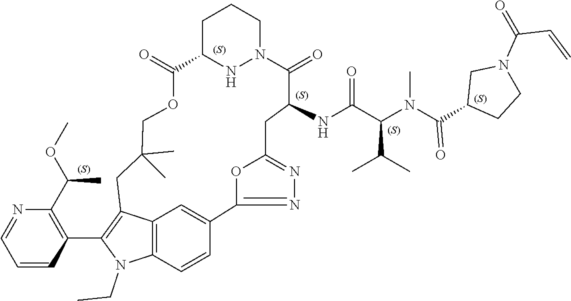 |
| A448 | 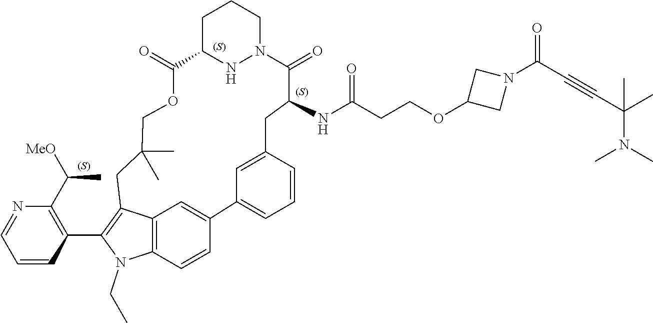 |
| A449 | 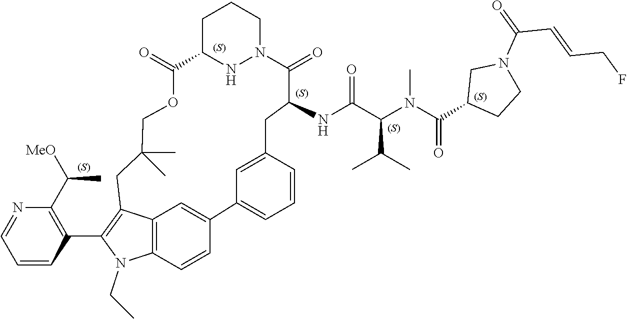 |
| A450 | 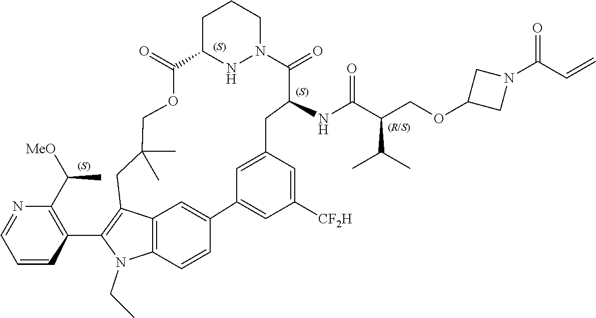 |
| A451 | 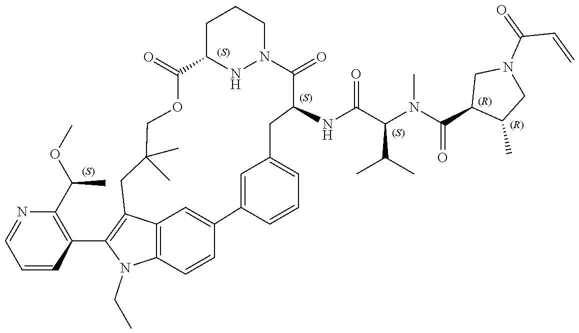 |
| A452 | 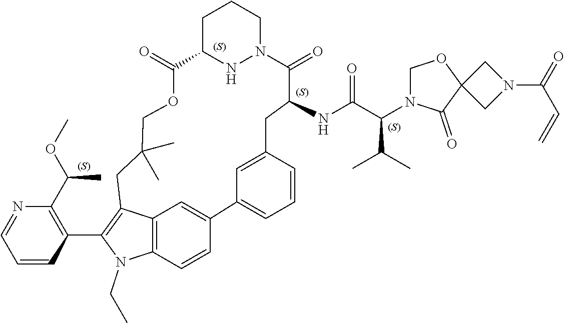 |
| A453 | 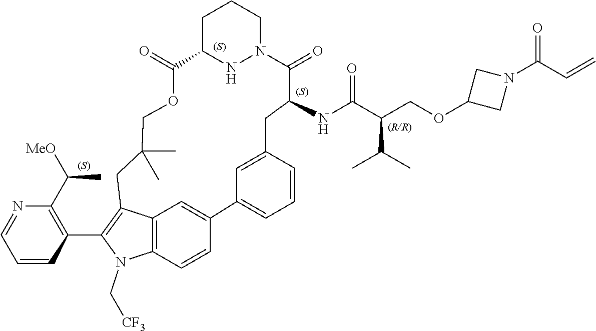 |
| A454 | 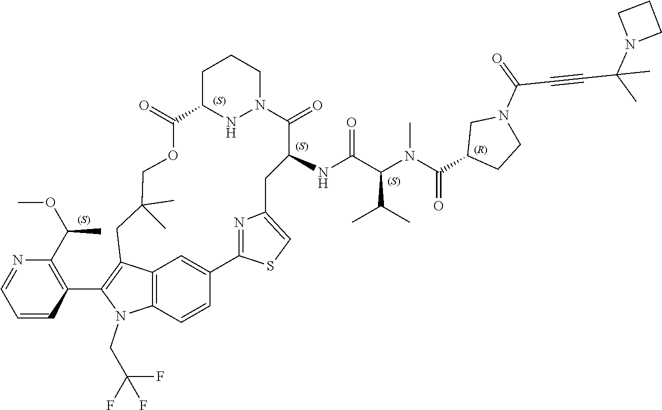 |
| A455 | 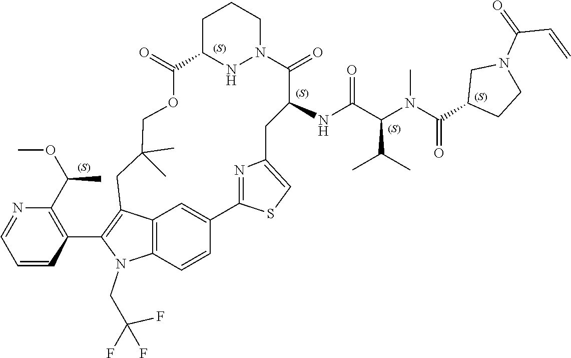 |
| A456 | 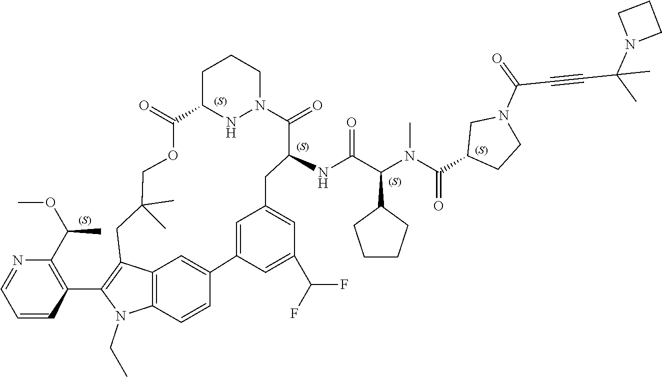 |
| A457 | 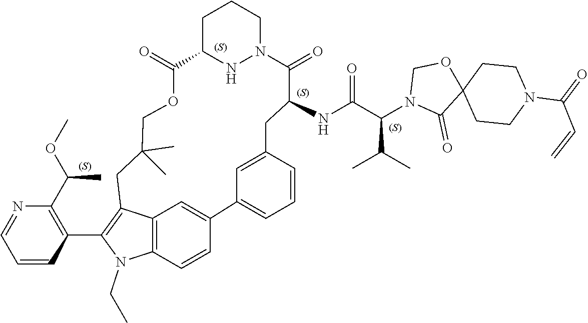 |
| A458 | 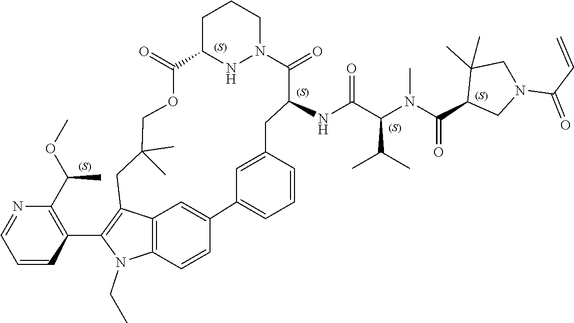 |
| A459 | 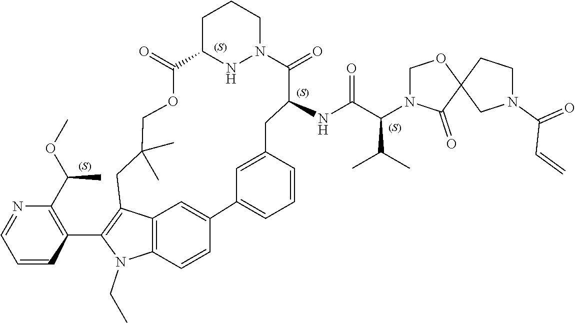 |
| A460 | 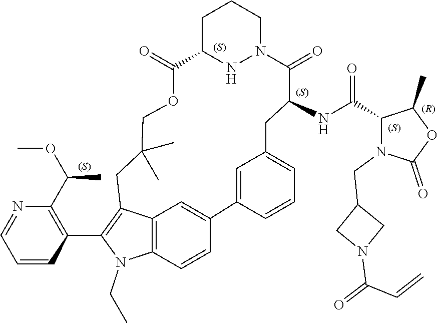 |
| A461 | 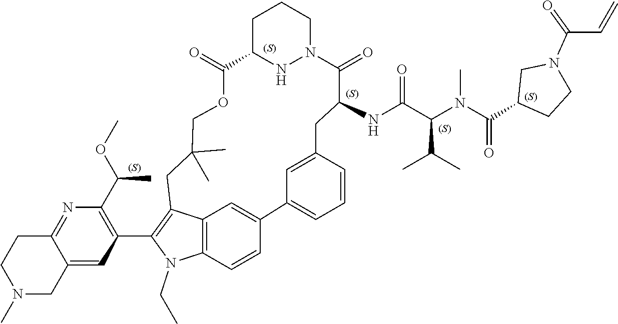 |
| A462 | 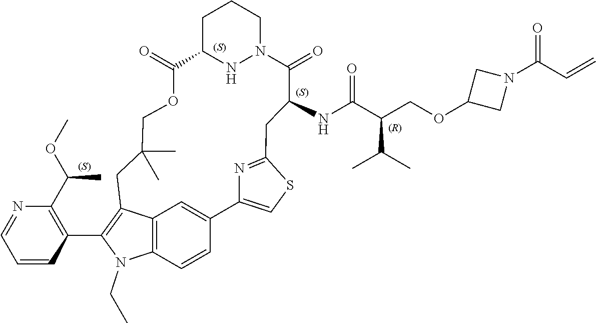 |
| A463 | 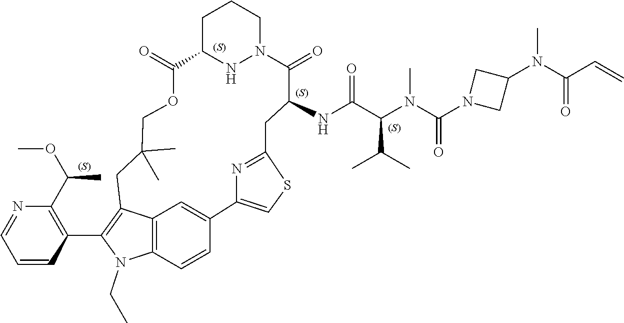 |
| A464 | 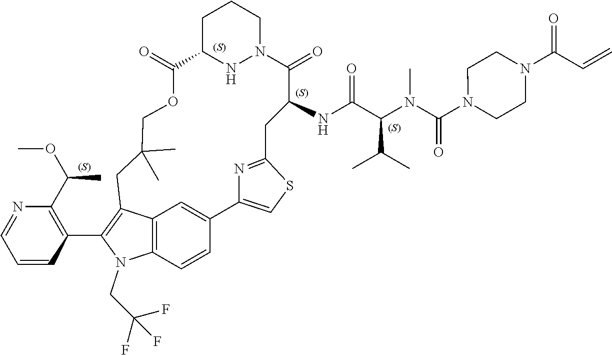 |
| A465 | 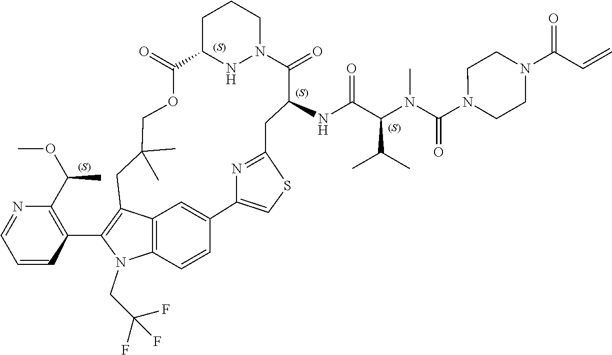 |
| A466 | 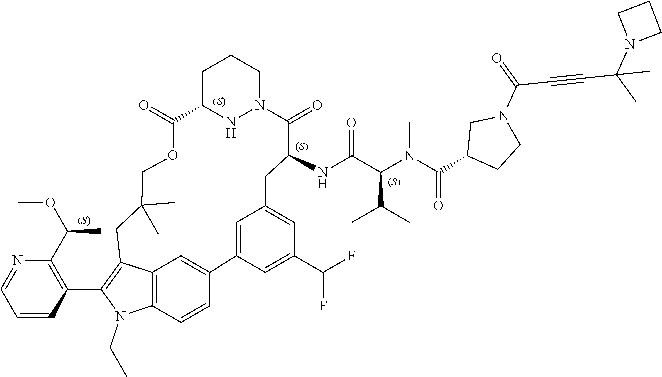 |
| A4467 | 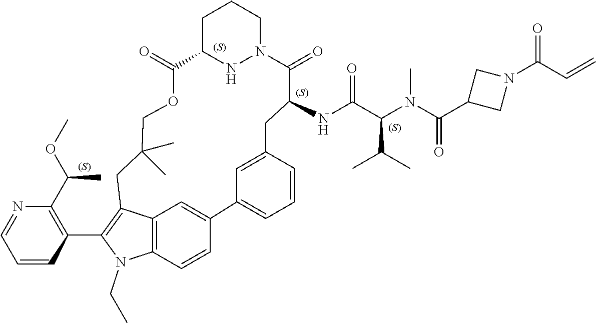 |
| A468 | 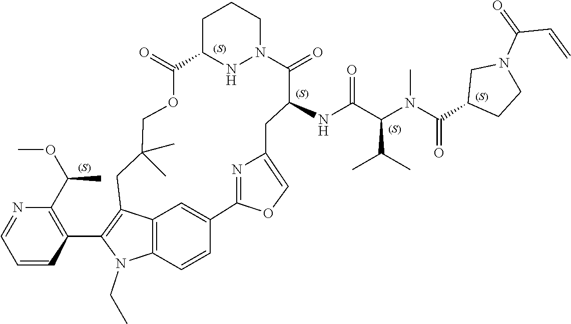 |
| A469 | 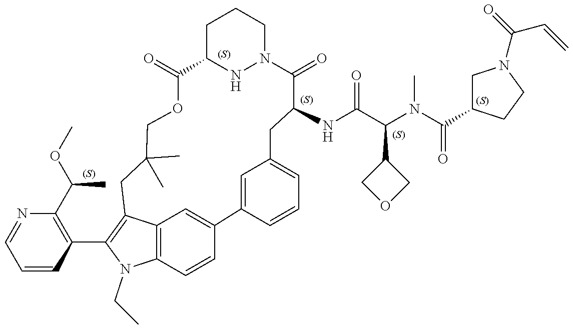 |
| A470 | 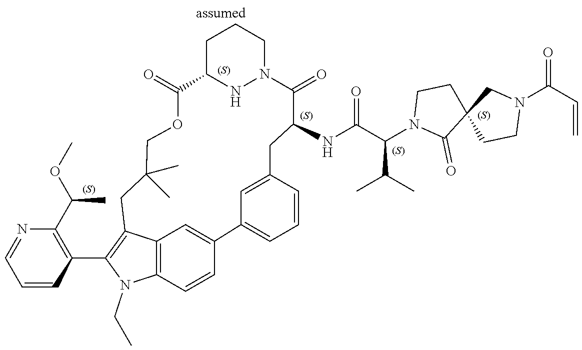 |
| A471 | 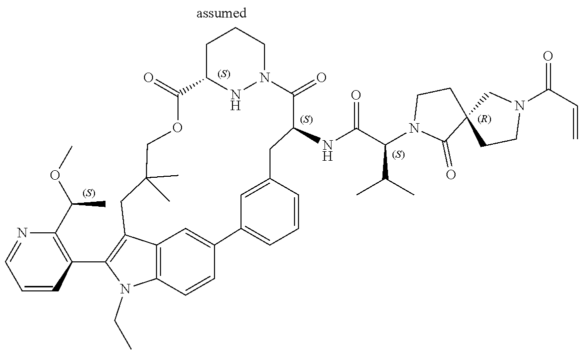 |
| A472 | 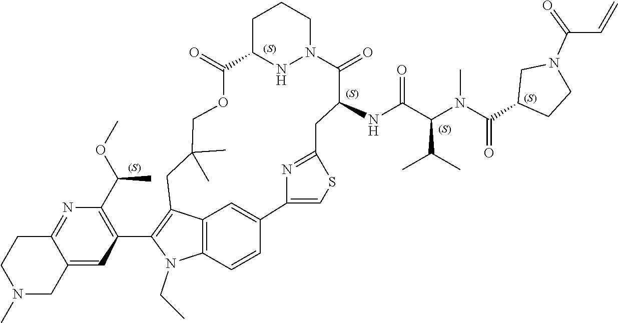 |
| A473 | 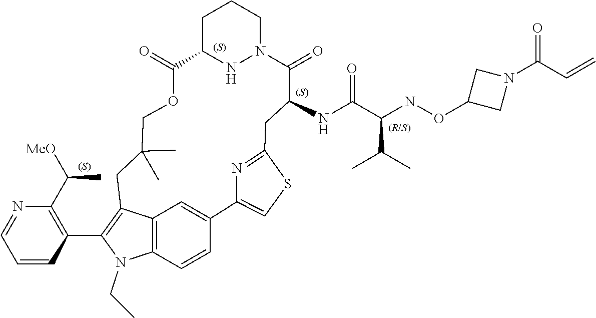 |
| A474 | 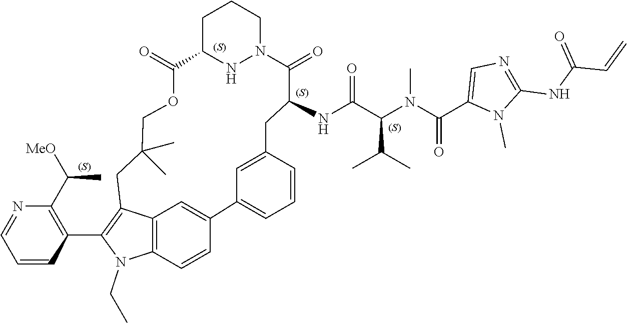 |
| A475 | 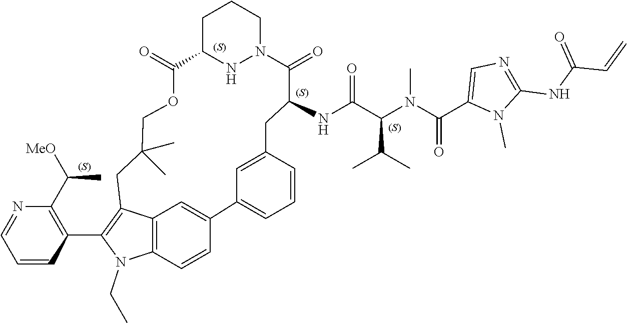 |
| A476 | 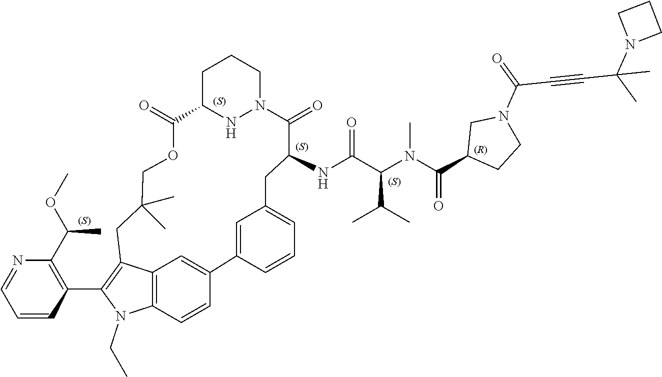 |
| A477 | 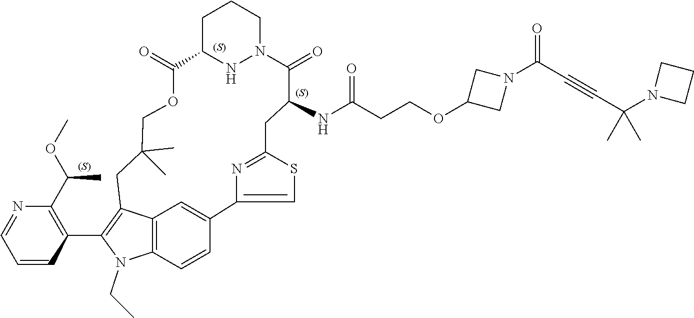 |
| A478 | 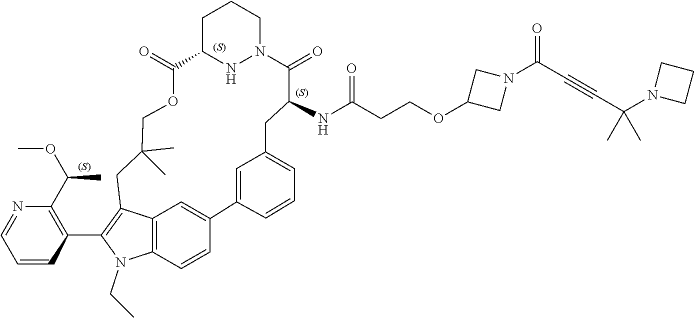 |
| A479 | 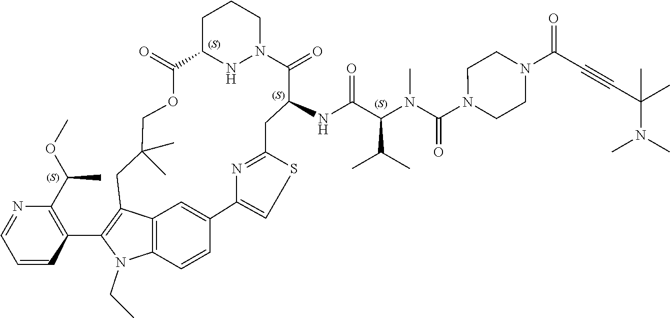 |
| A480 | 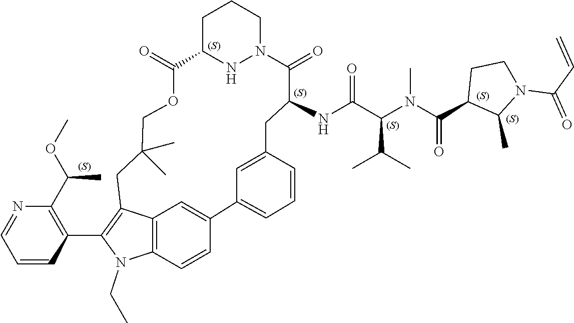 |
| A481 | 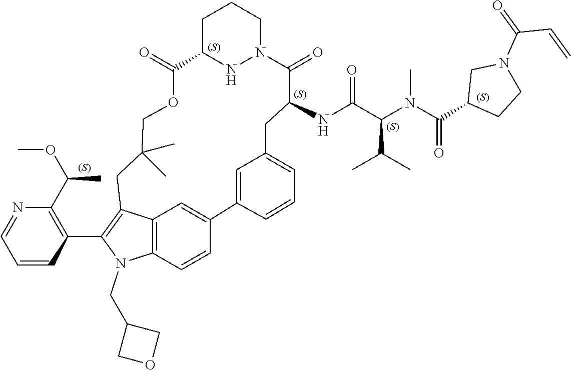 |
| A482 | 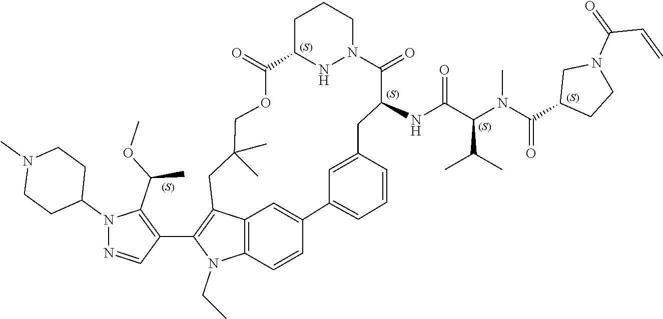 |
| A483 | 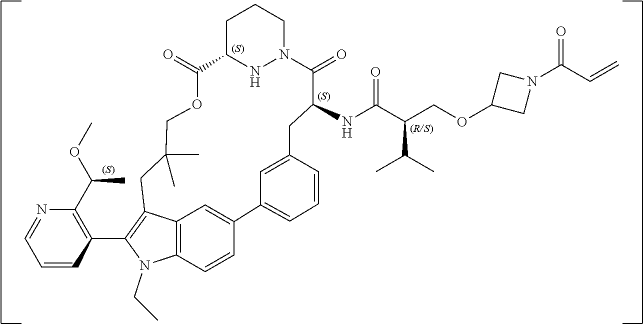 |
| A484 | 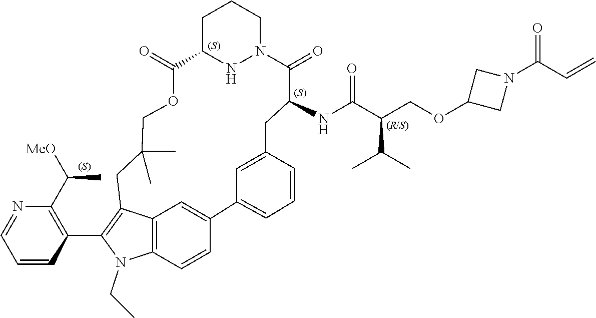 |
| A485 | 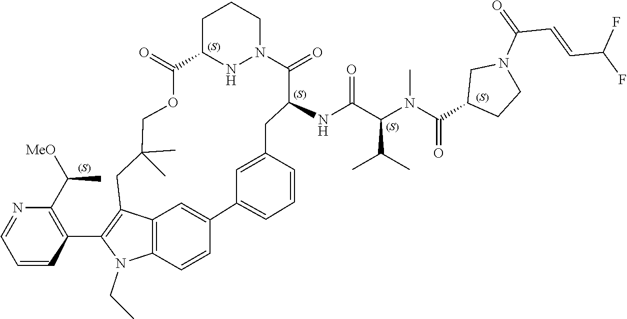 |
| A486 | 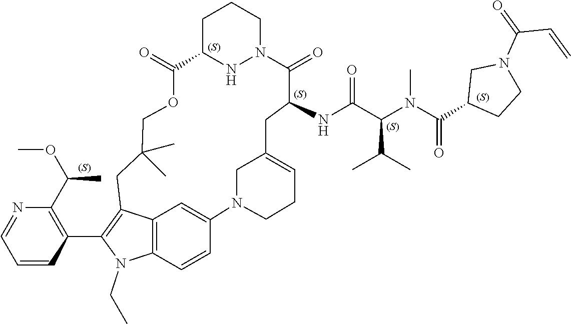 |
| A487 | 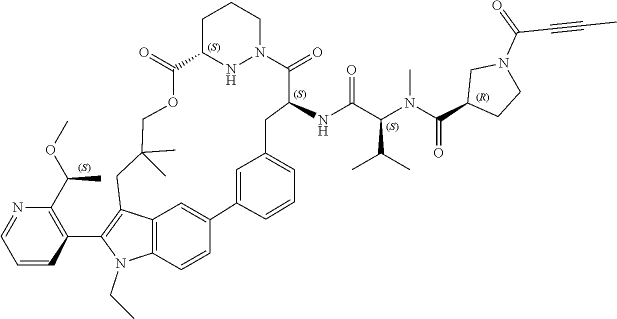 |
| A488 | 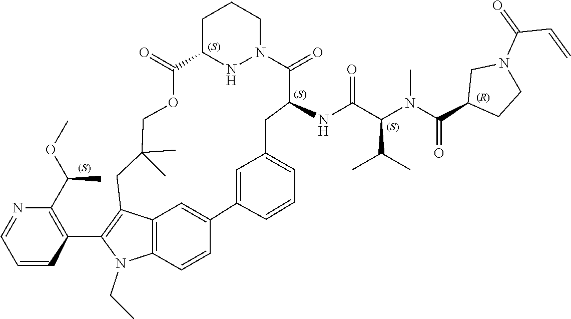 |
| A489 | 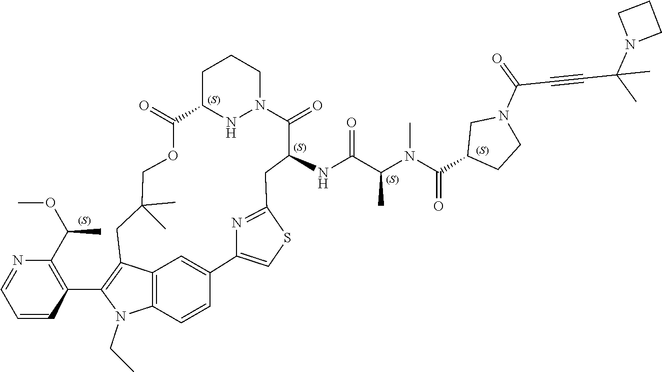 |
| A490 | 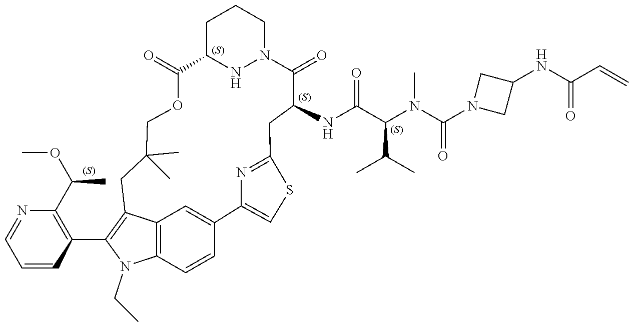 |
| A491 | 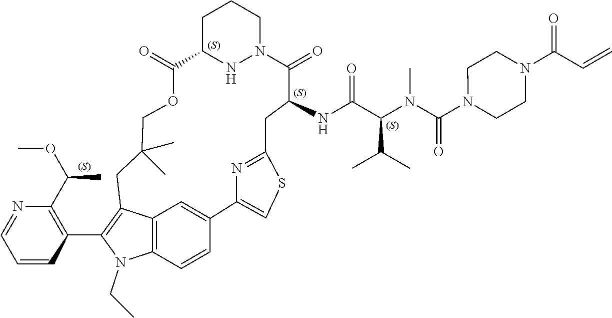 |
| A492 | 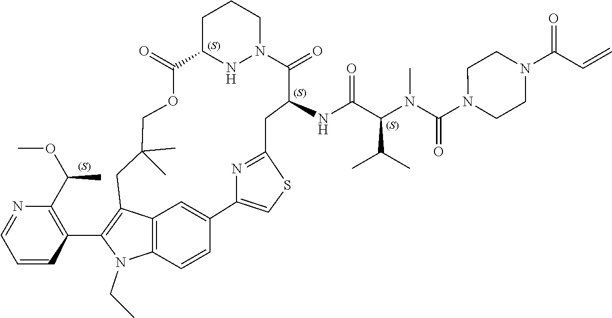 |
| A493 | 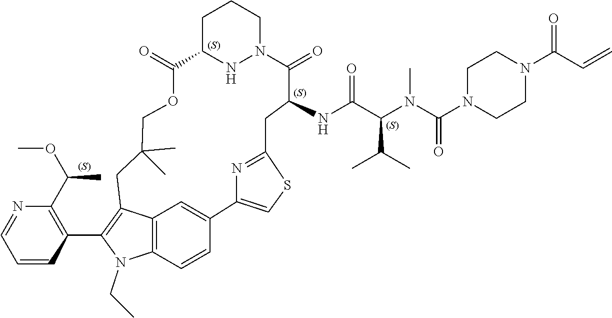 |
| A494 | 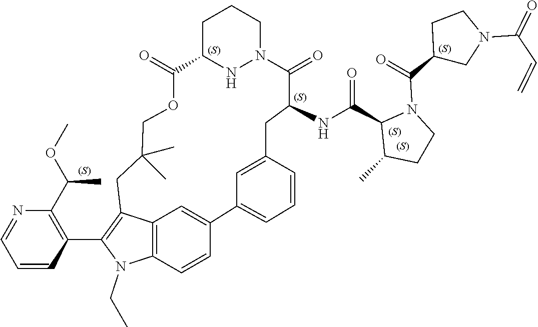 |
| A495 | 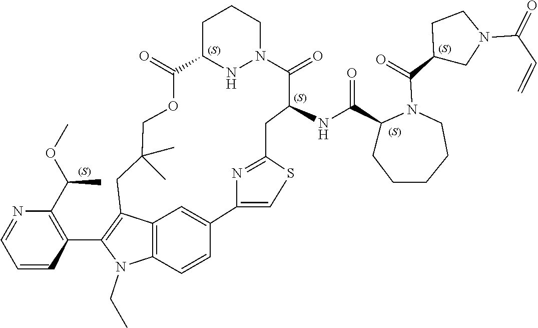 |
| A496 | 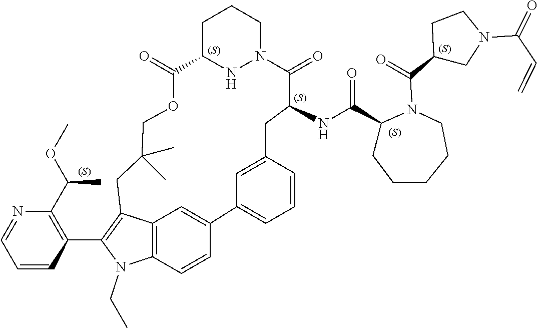 |
| A497 | 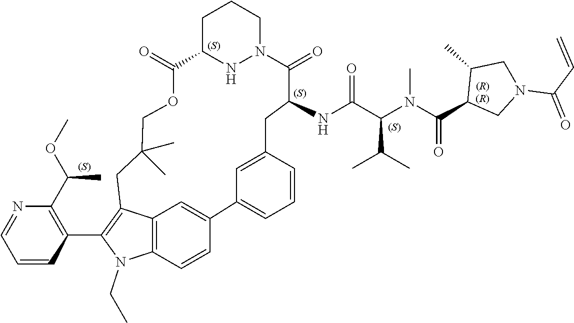 |
| A498 | 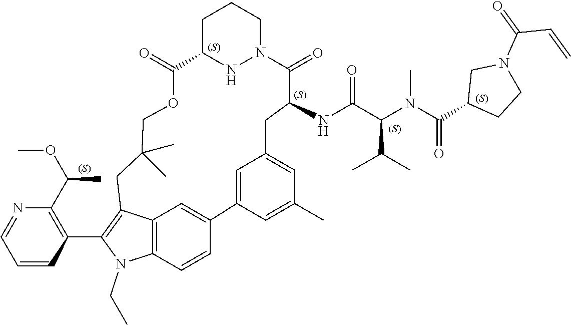 |
| A499 | 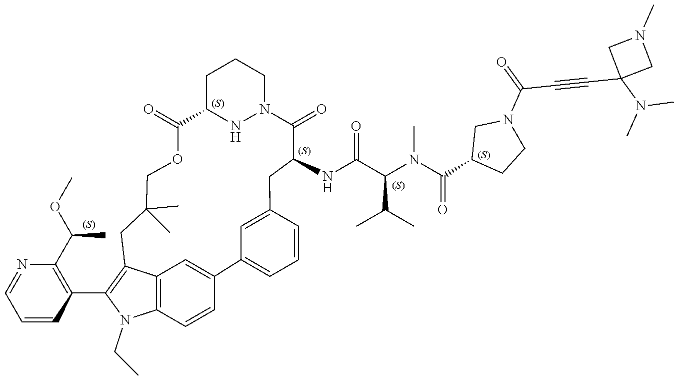 |
| A500 | 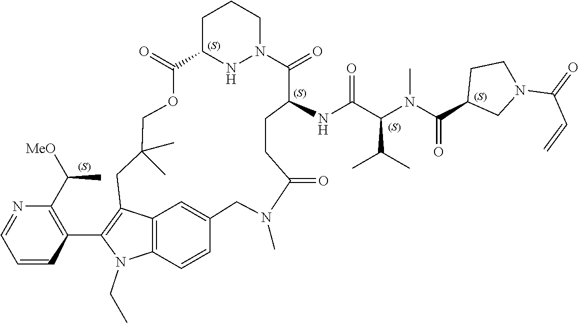 |
| A501 | 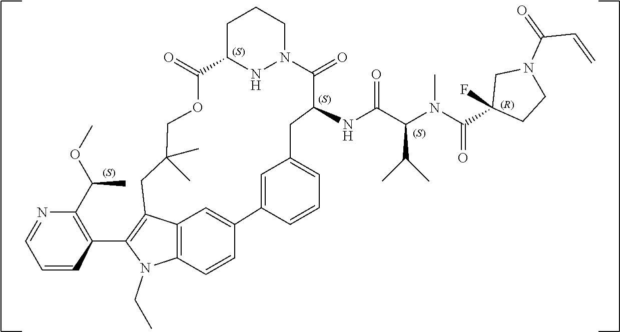 |
| A502 | 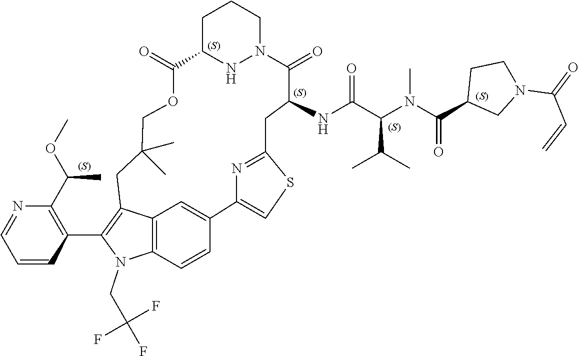 |
| A503 | 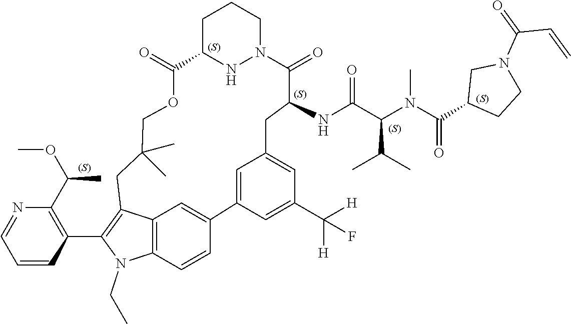 |
| A504 | 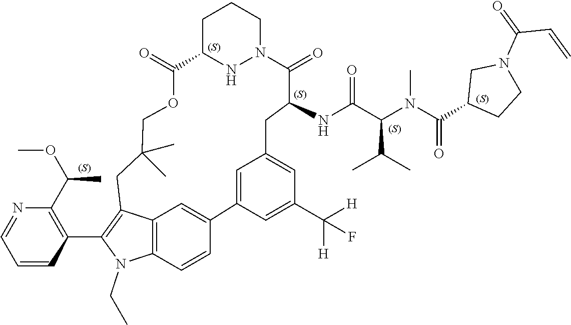 |
| A505 | 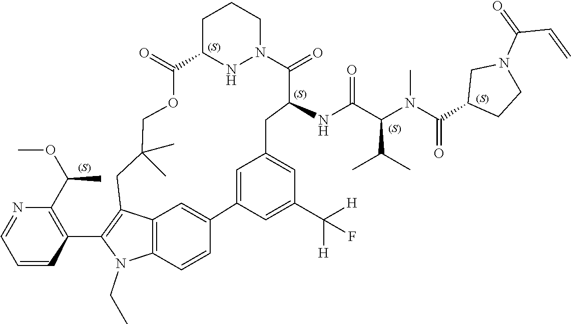 |
| A506 | 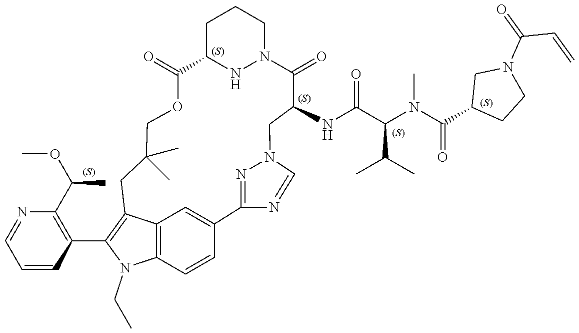 |
| A507 | 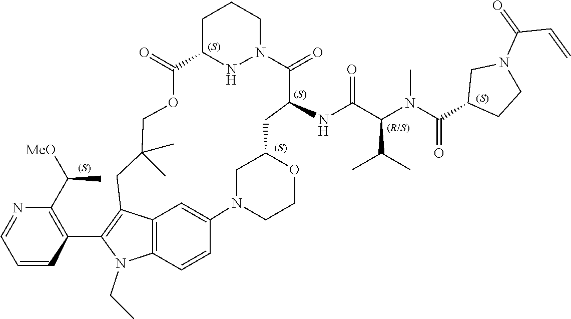 |
| A508 | 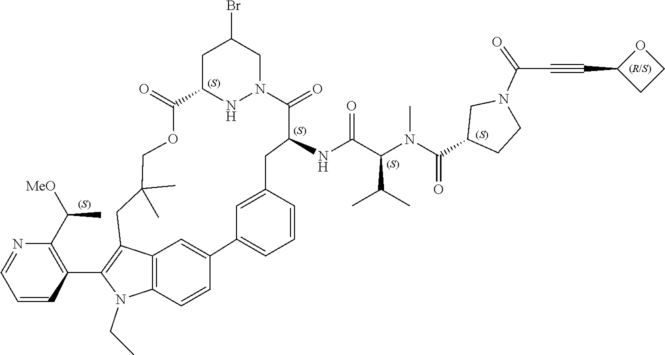 |
| A509 | 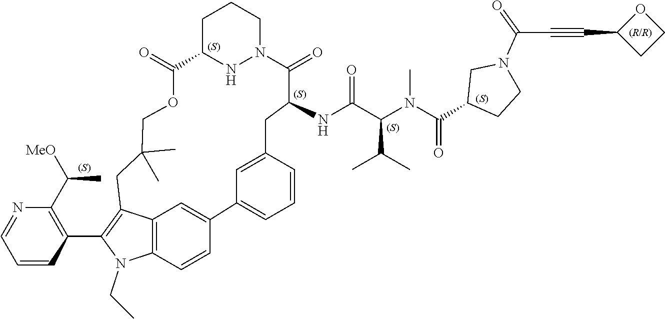 |
| A510 | 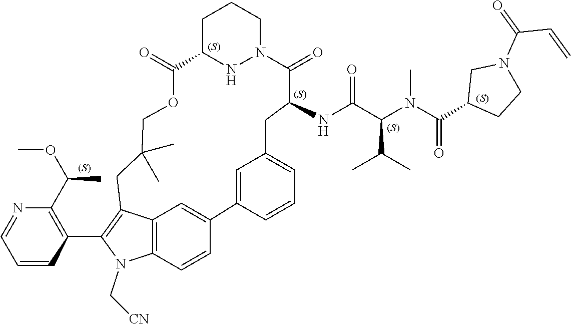 |
| A511 | 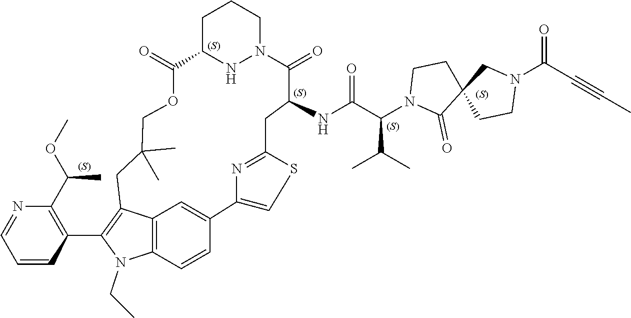 |
| A512 | 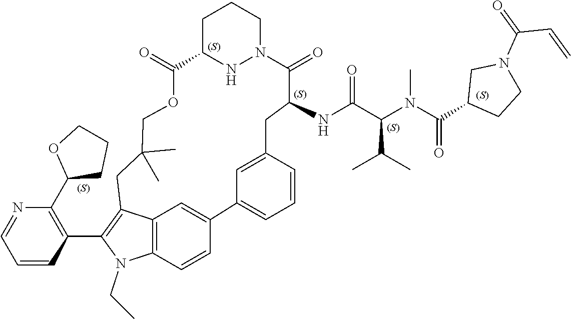 |
| A513 | 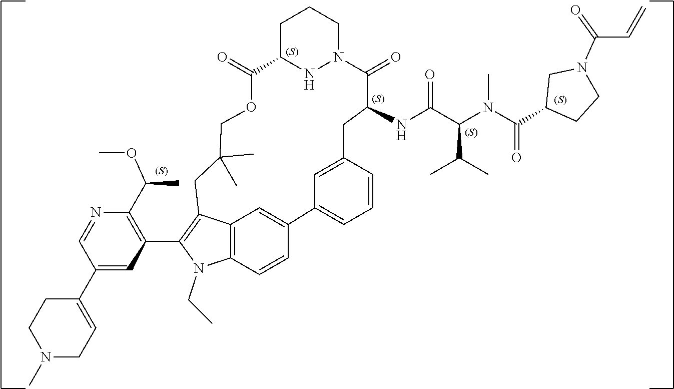 |
| A514 | 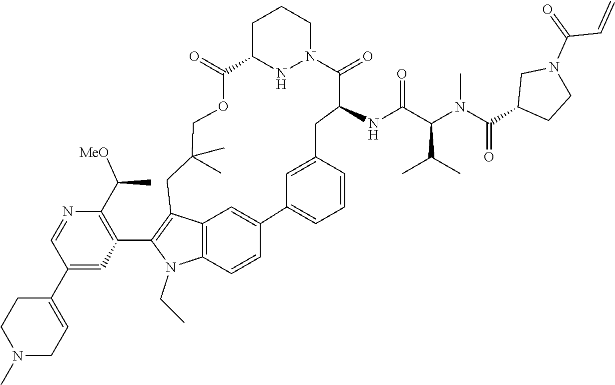 |
| A515 | 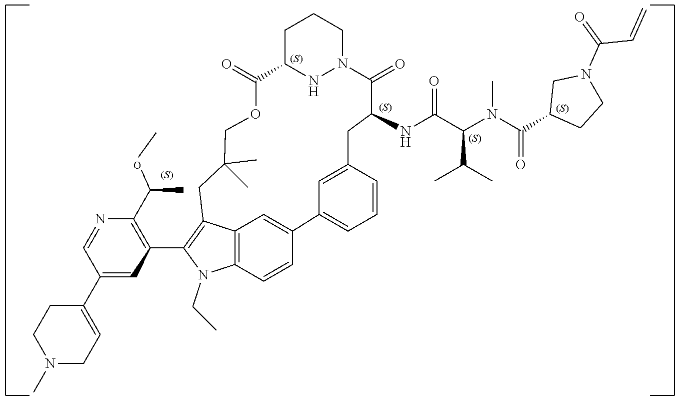 |
| A516 | 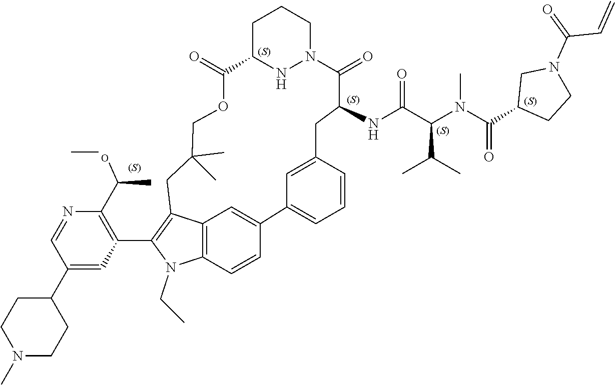 |
| A517 | 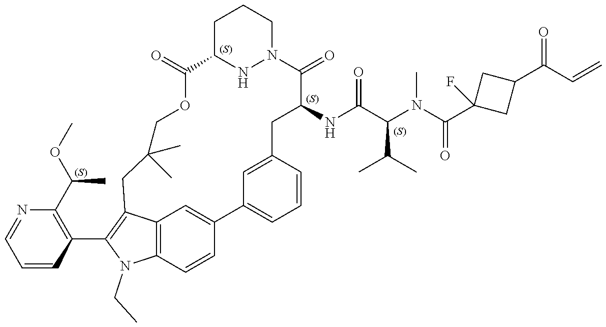 |
| A518 | 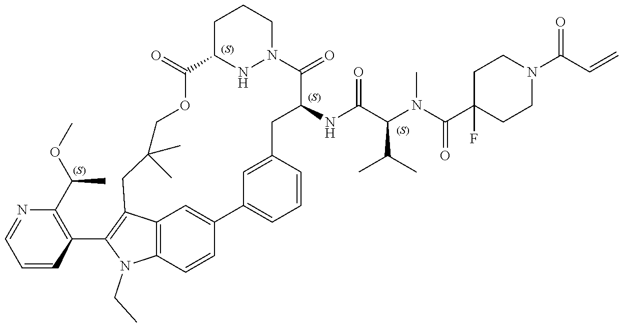 |
| A519 | 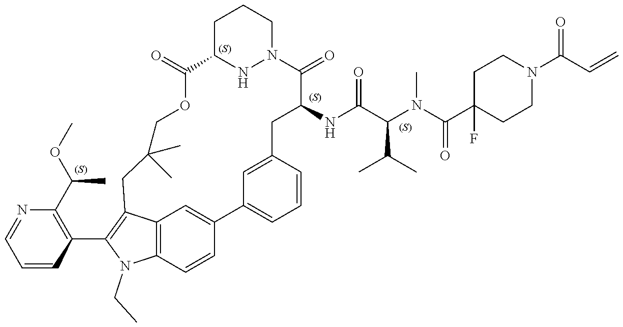 |
| A520 | 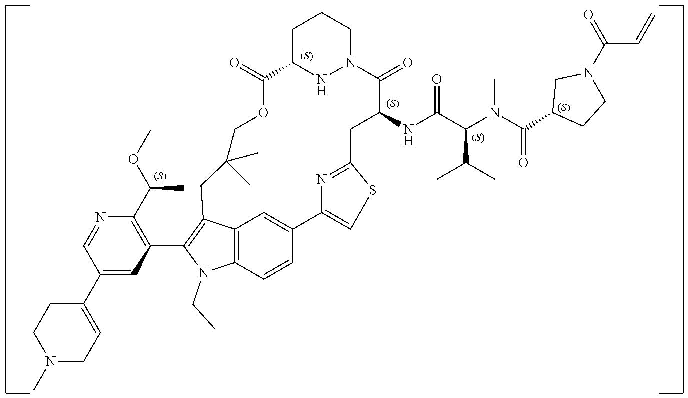 |
| A521 | 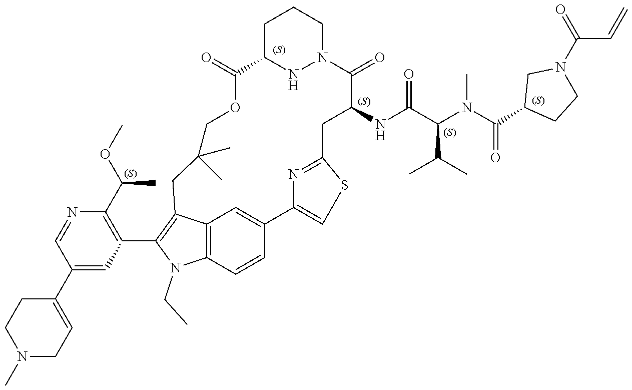 |
| A522 | 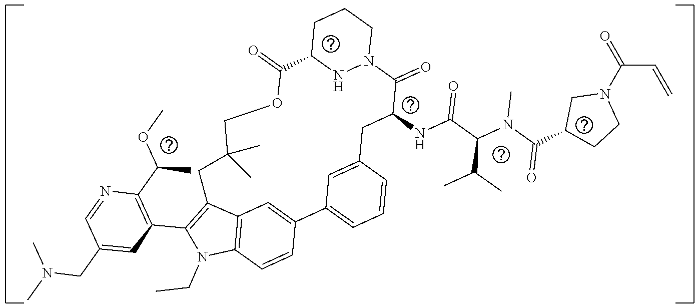 |
| A523 | 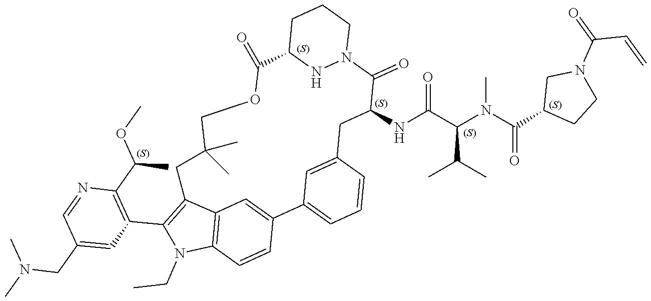 |
| A524 | 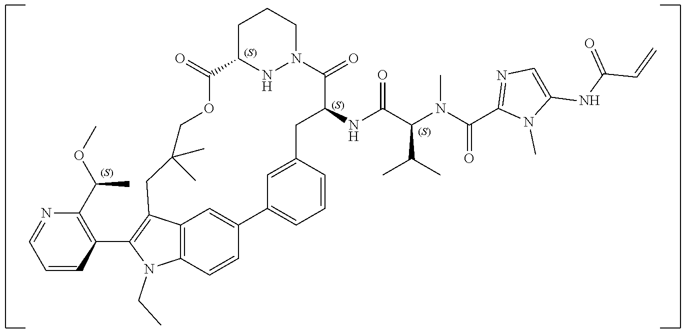 |
| A525 | 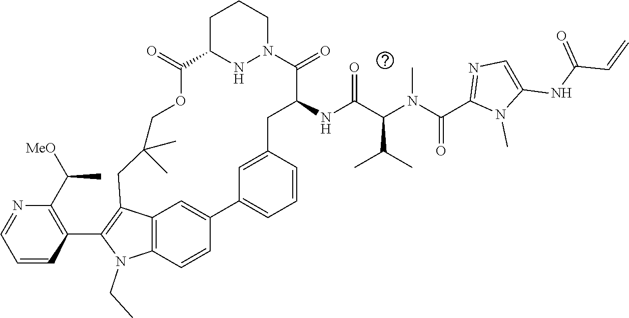 |
| A526 | 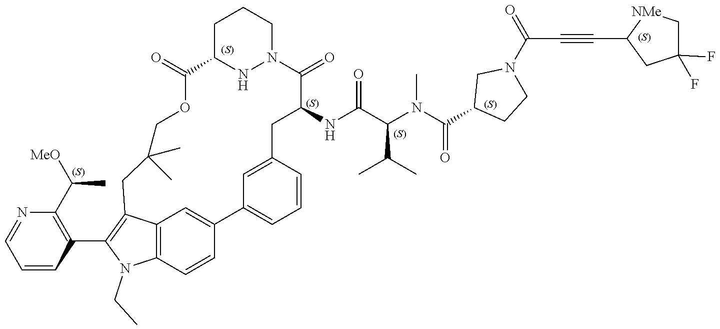 |
| A527 | 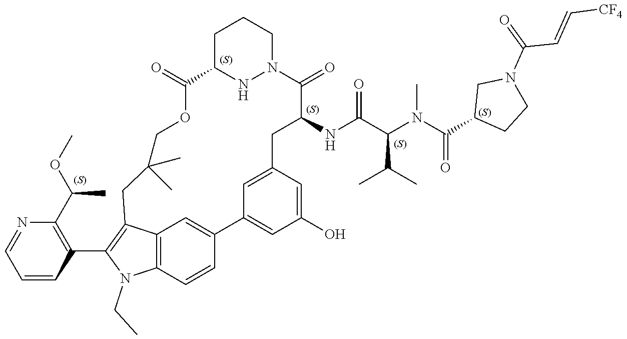 |
| A528 | 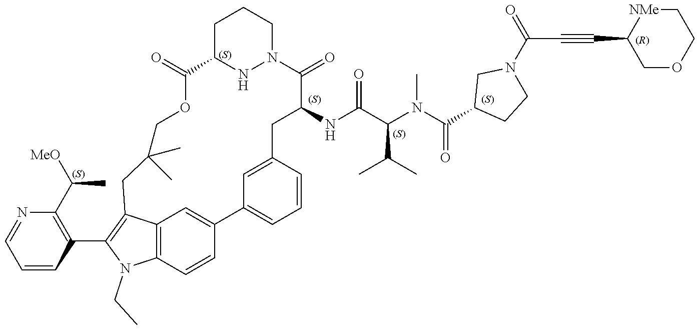 |
| A529 | 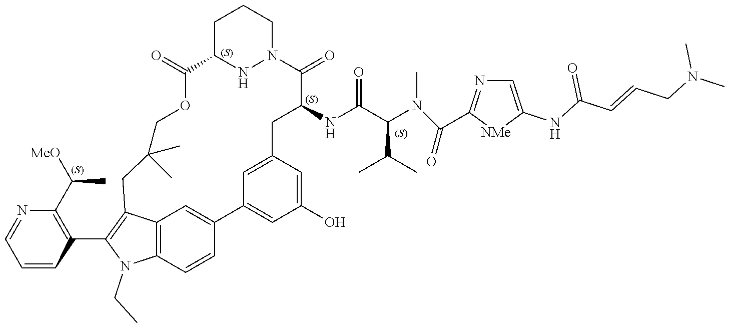 |
| A530 | 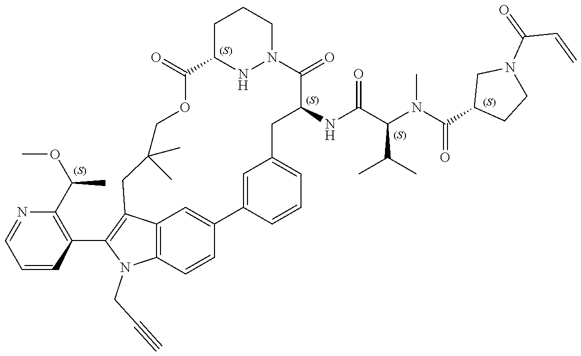 |
| A531 | 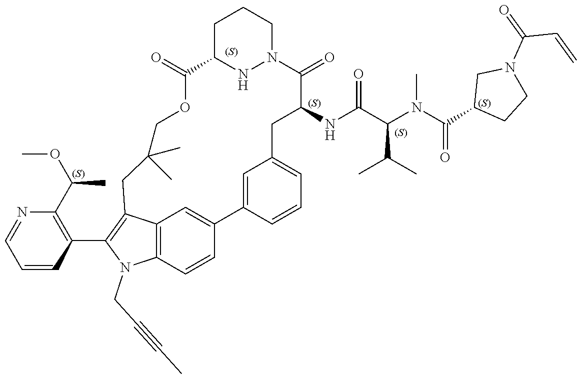 |
| A532 | 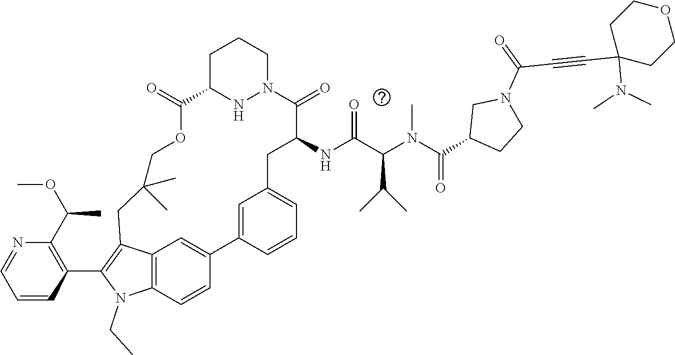 |
| A533 | 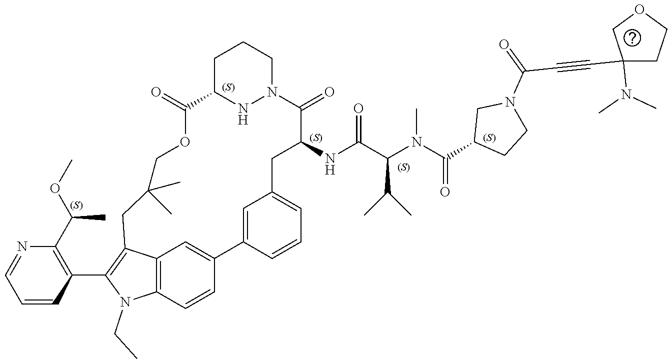 |
| A534 | 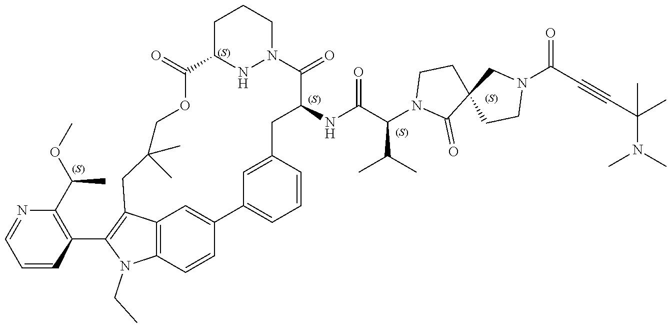 |
| A535 | 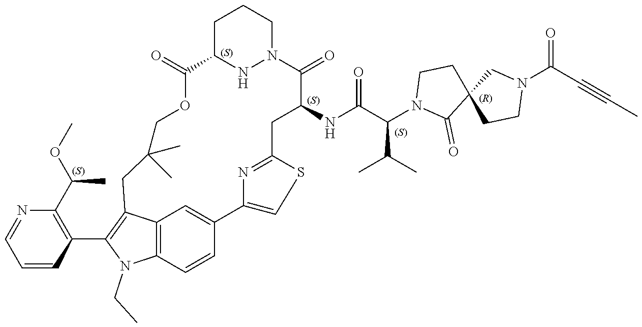 |
| A536 | 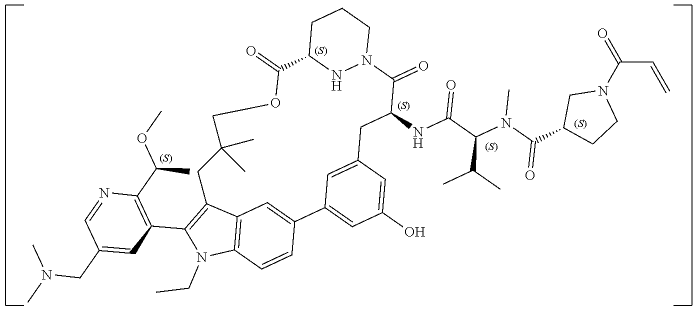 |
| A537 | 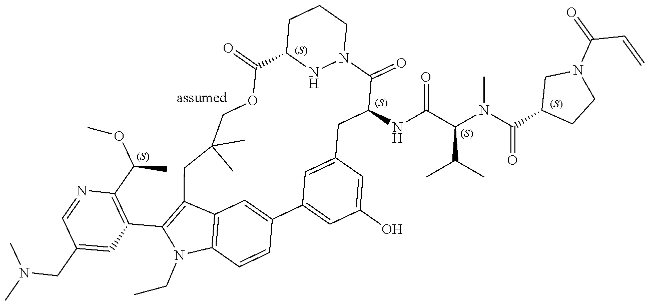 |
| A538 | 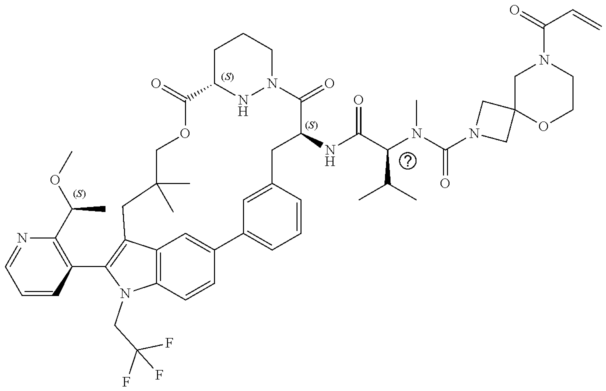 |
| A539 | 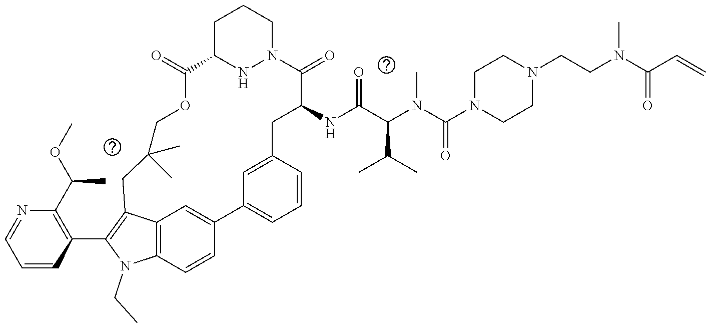 |
| A540 | 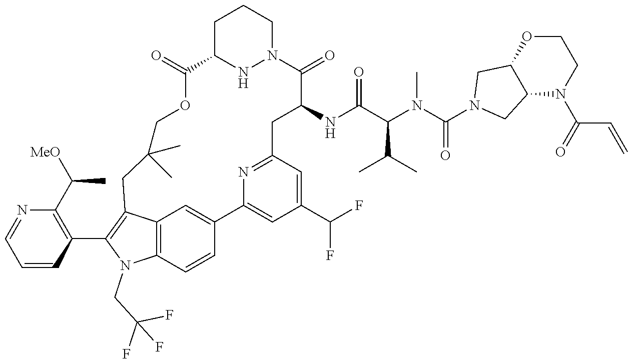 |
| A541 | 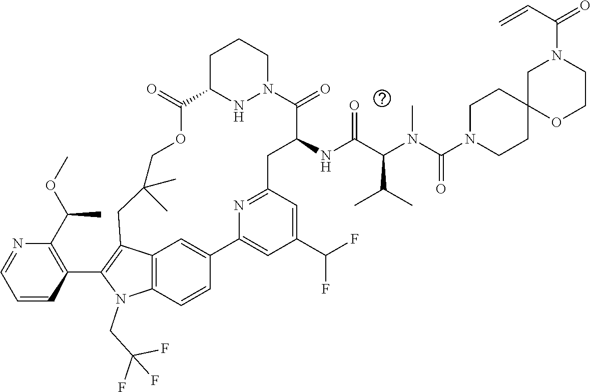 |
| A542 | 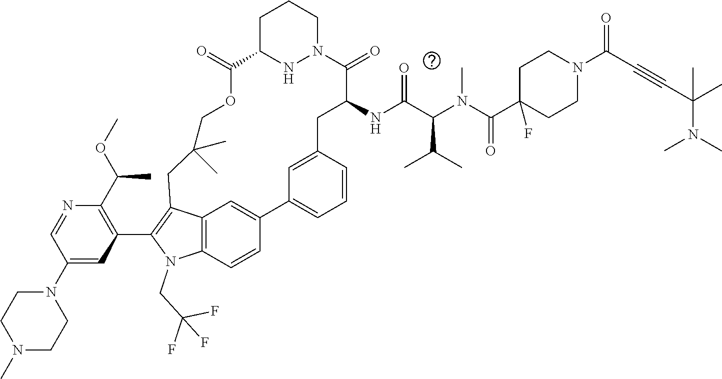 |
| A543 | 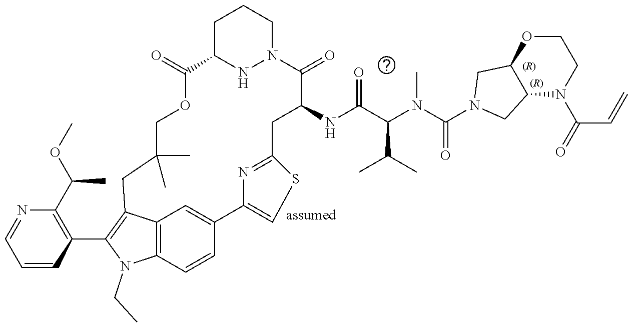 |
| A544 | 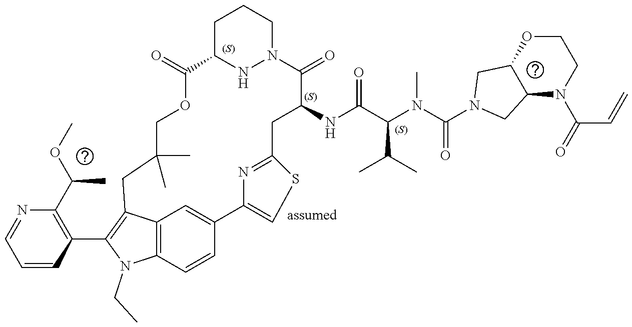 |
| A545 | 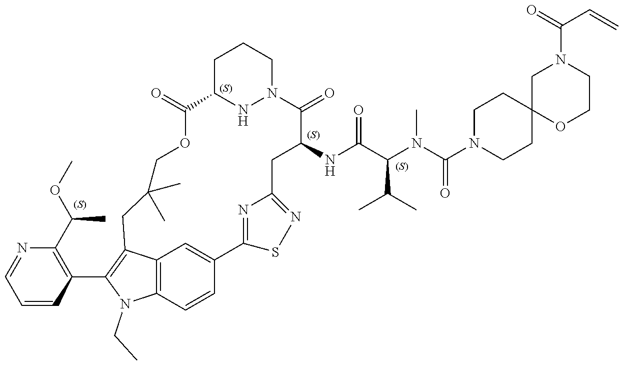 |
| A546 | 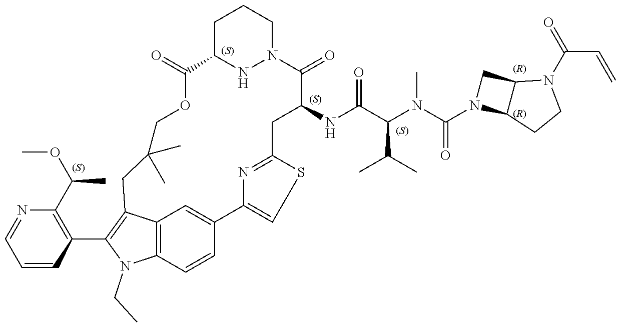 |
| A547 | 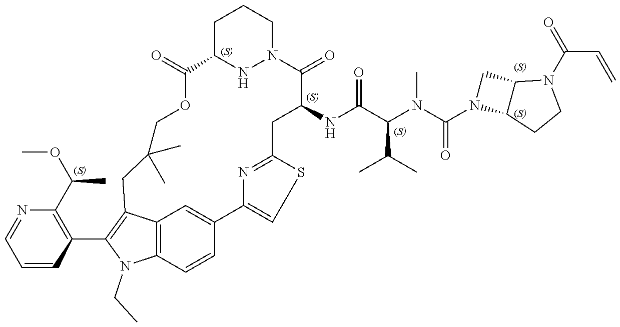 |
| A548 | 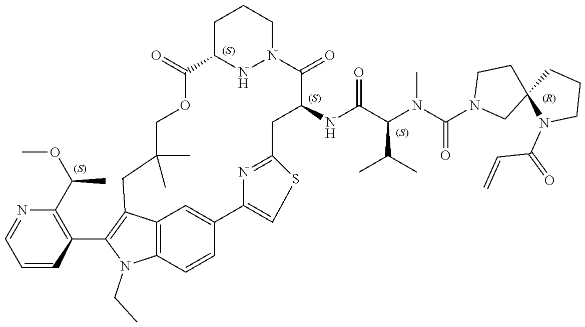 |
| A549 | 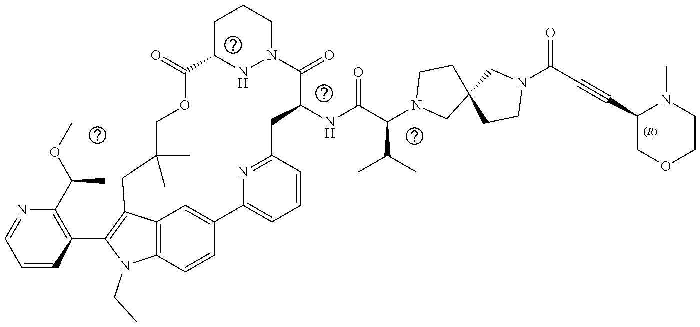 |
| A550 | 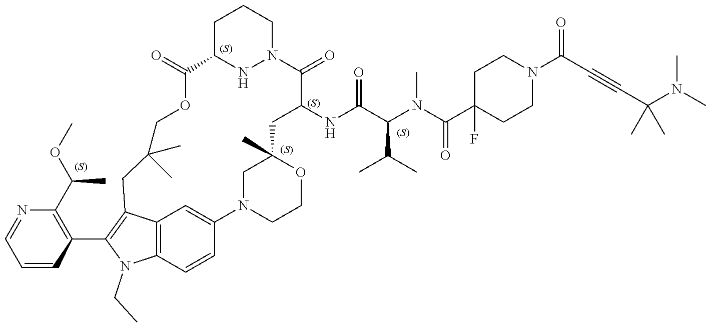 |
| A551 | 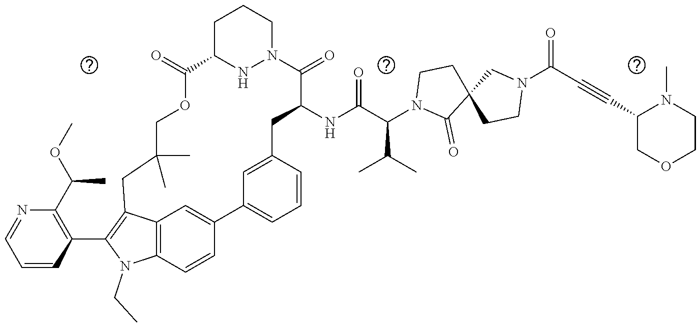 |
| A552 | 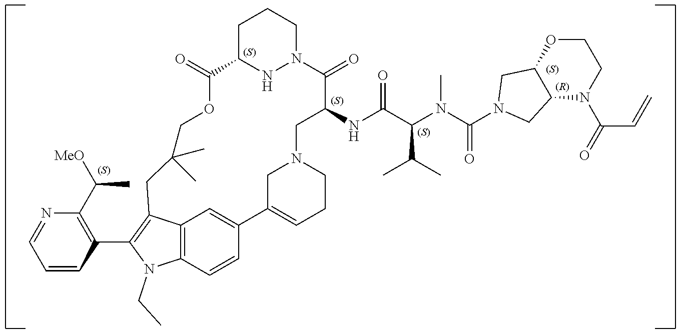 |
| A553 | 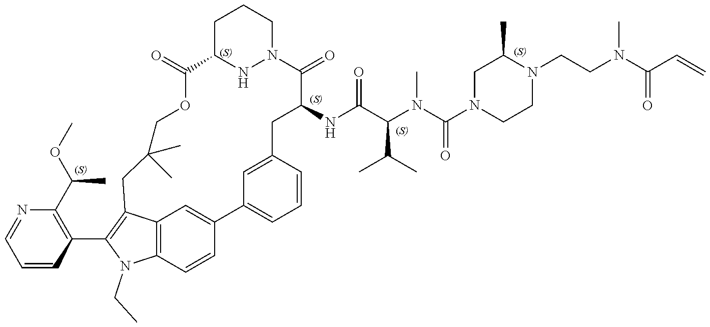 |
| A554 | 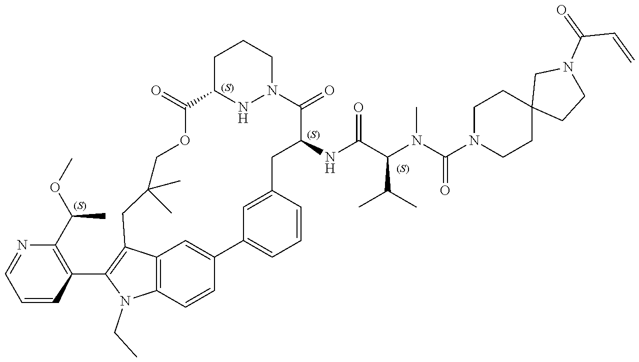 |
| A555 | 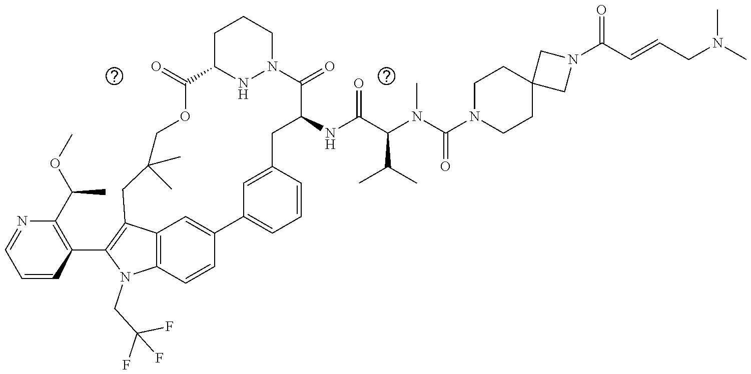 |
| A556 | 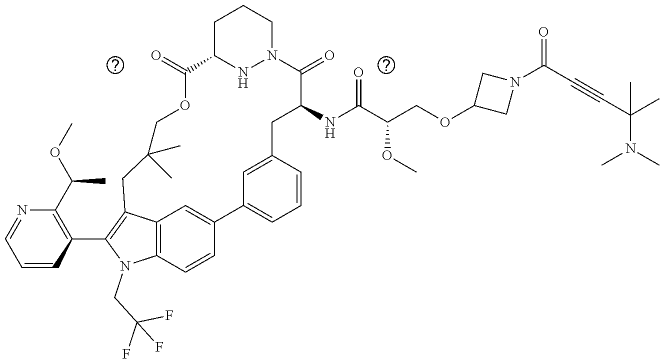 |
| A557 | 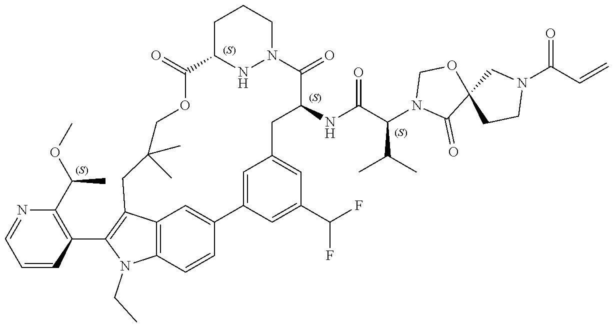 |
| A558 | 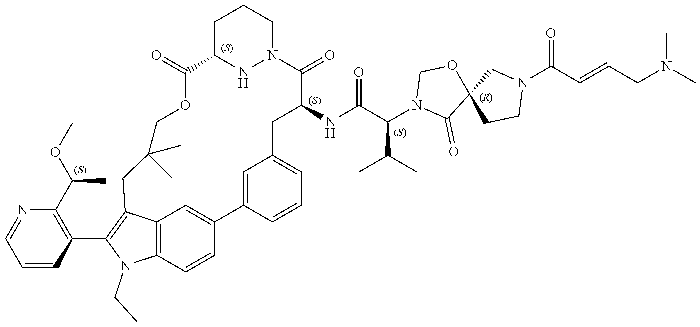 |
| A559 | 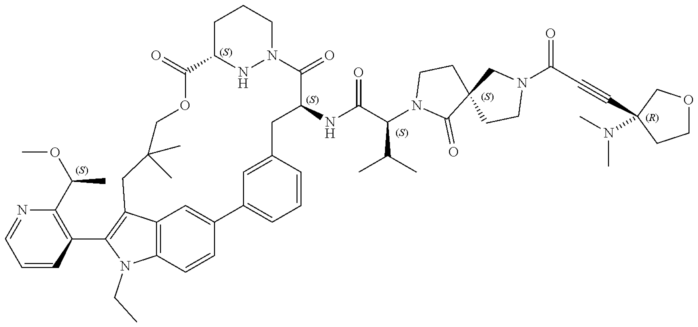 |
| A560 | 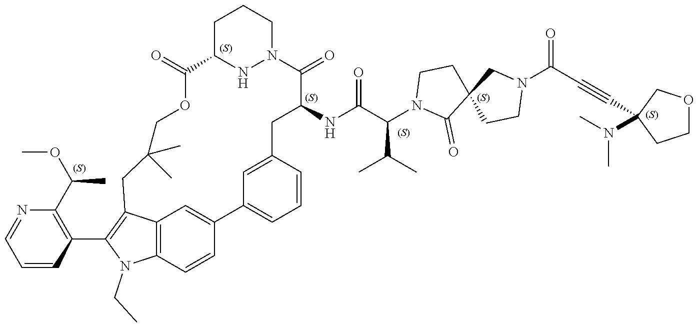 |
| A561 | 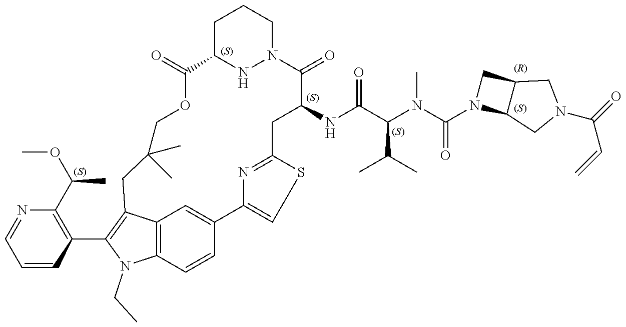 |
| A562 | 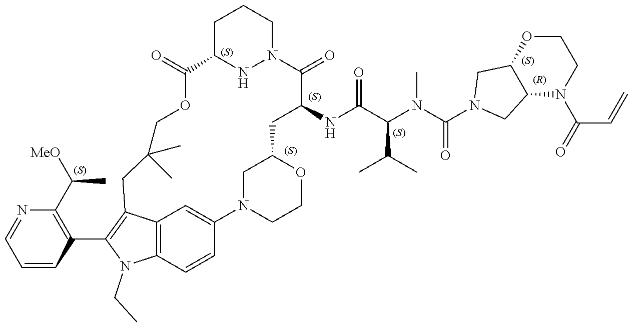 |
| A563 | 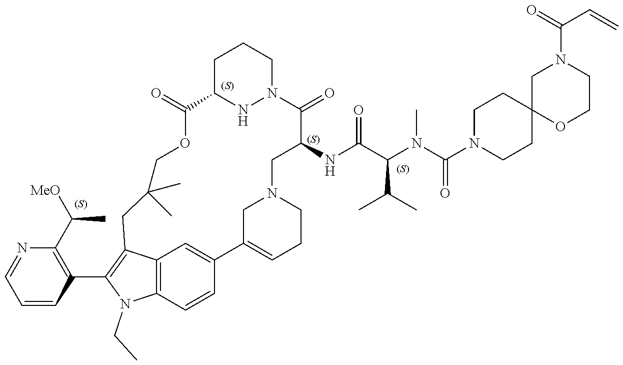 |
| A564 | 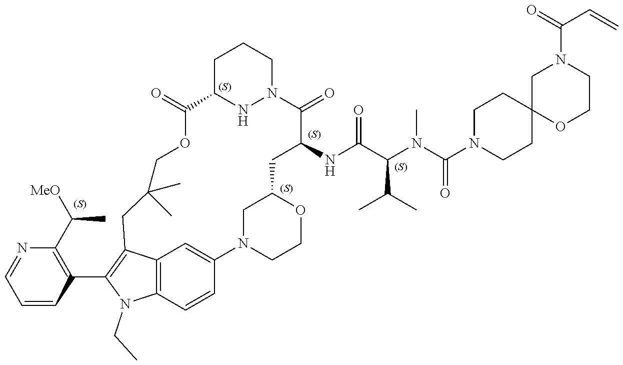 |
| A565 | 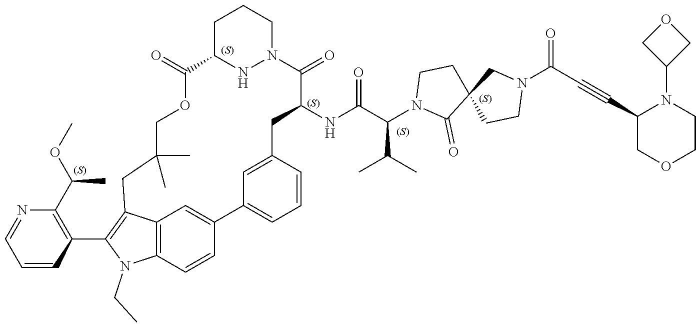 |
| A566 | 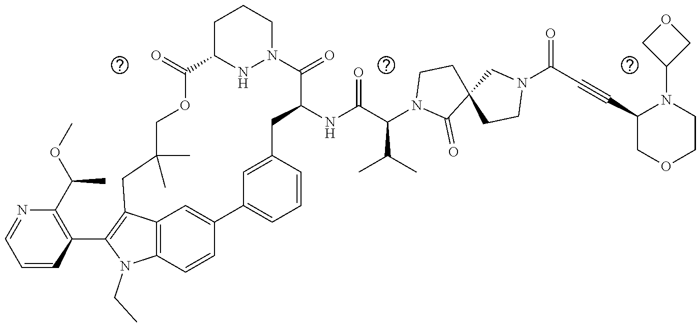 |
| A567 | 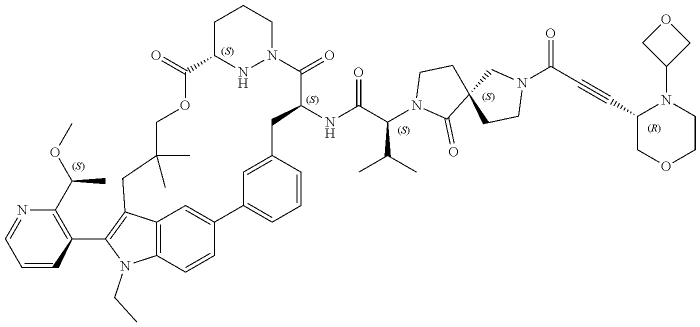 |
| A568 | 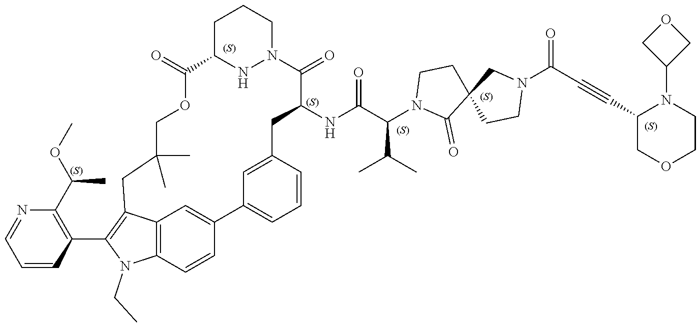 |
| A569 | 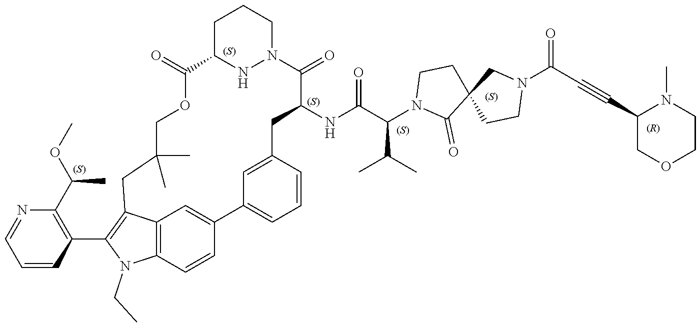 |
| A570 | 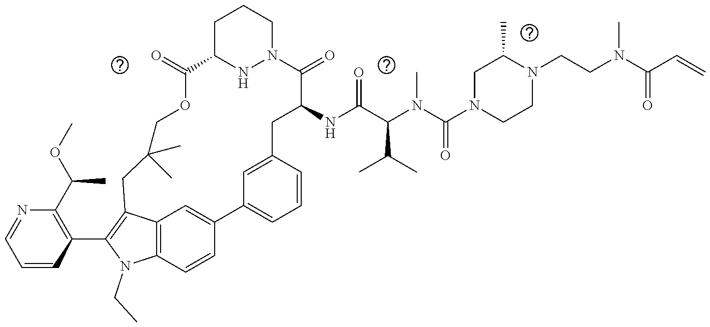 |
| A571 | 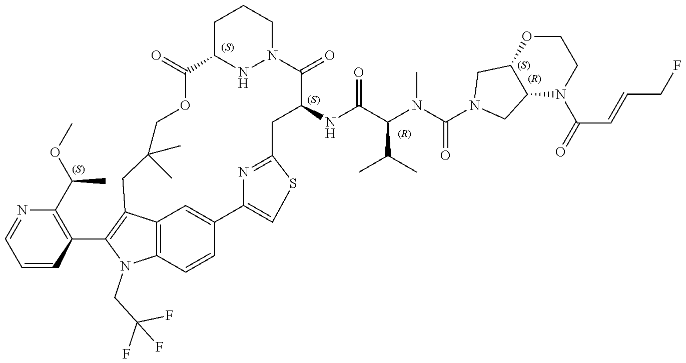 |
| A572 | 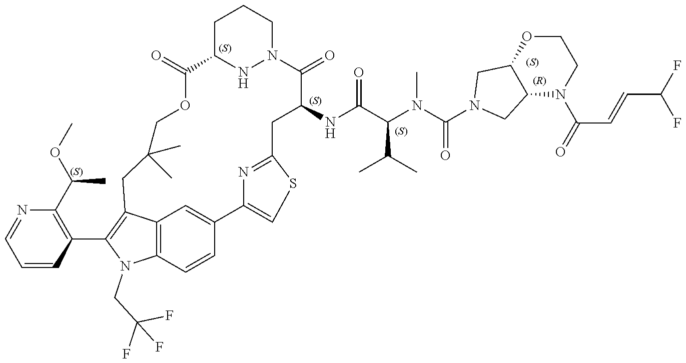 |
| A573 | 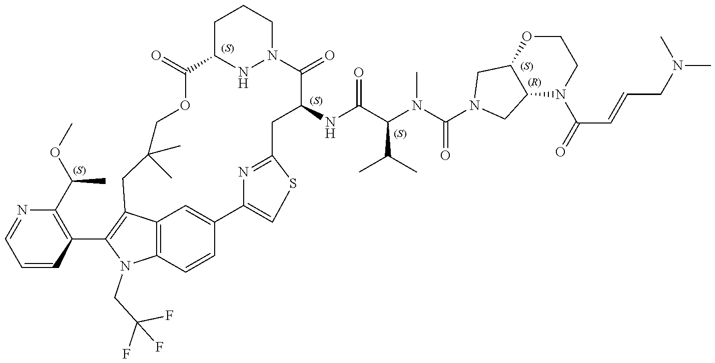 |
| A574 | 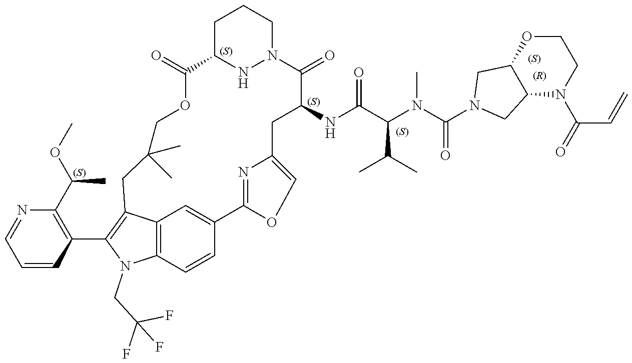 |
| A575 | 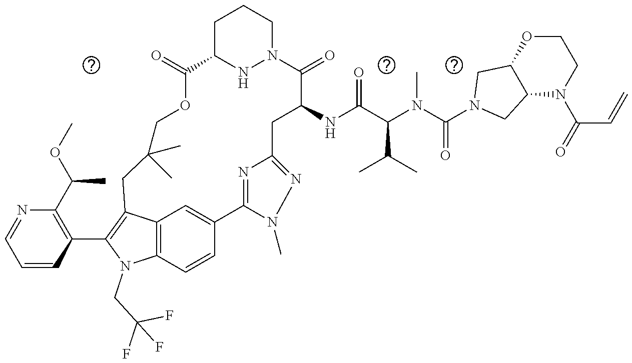 |
| A576 | 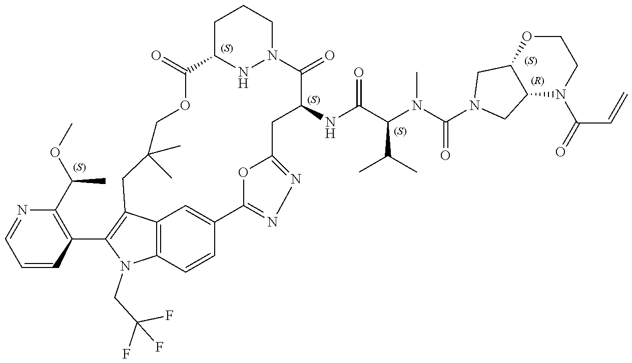 |
| A577 | 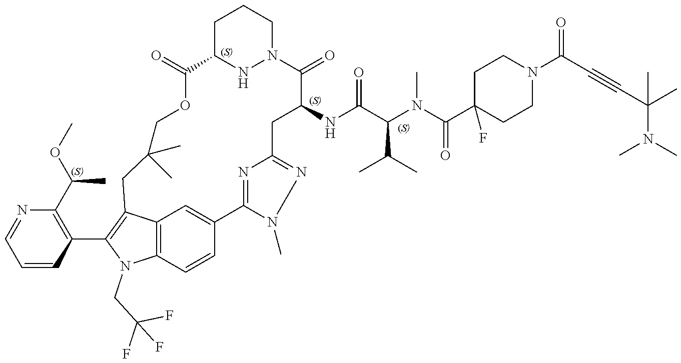 |
| A578 | 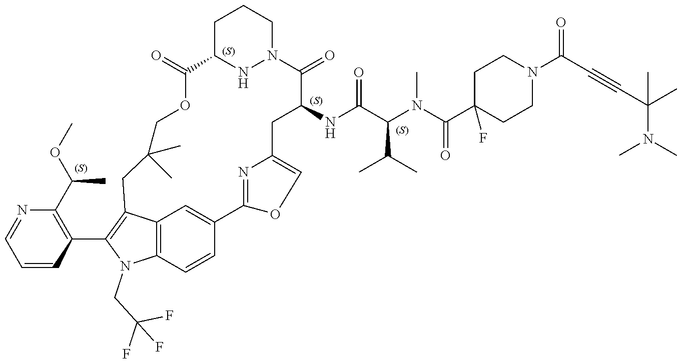 |
| A579 | 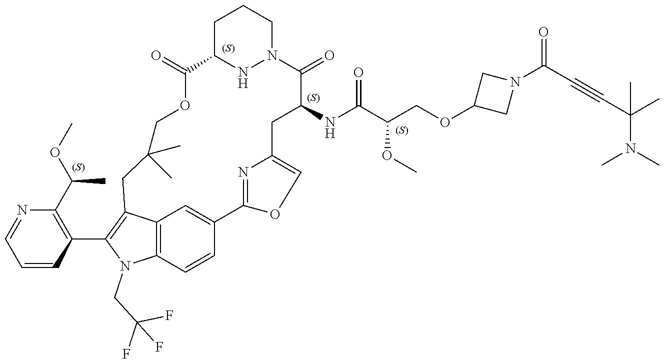 |
| A580 | 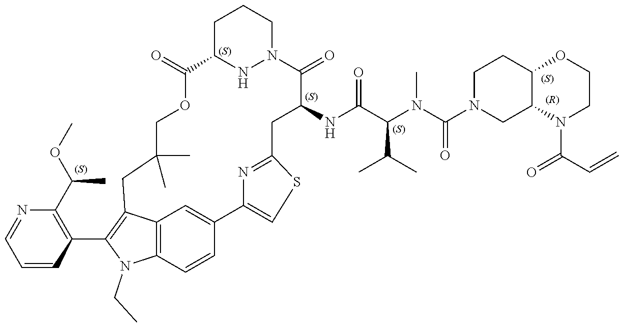 |
| A581 | 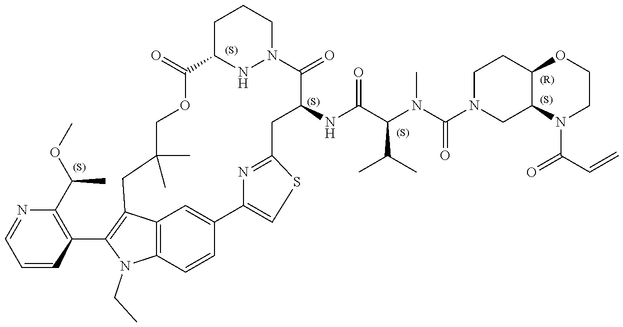 |
| A582 | 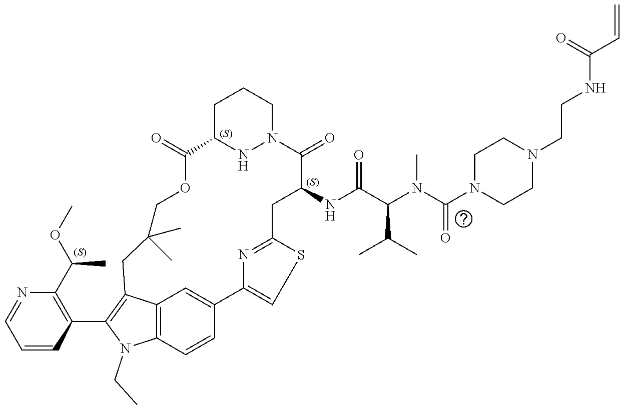 |
| A583 | 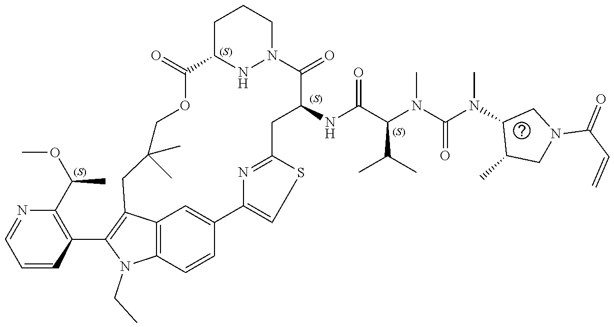 |
| A584 | 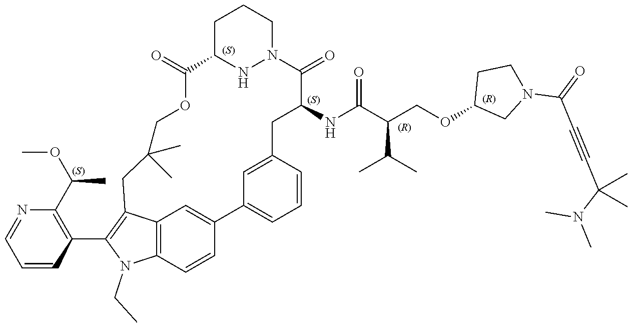 |
| A585 | 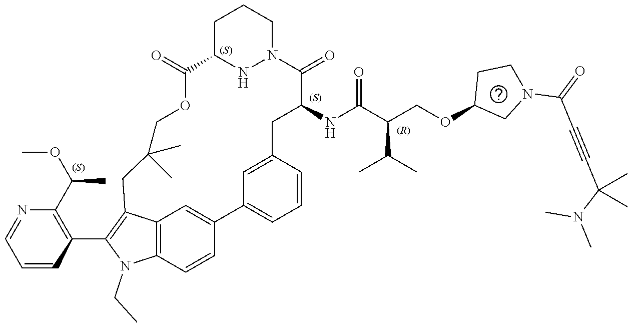 |
| A586 | 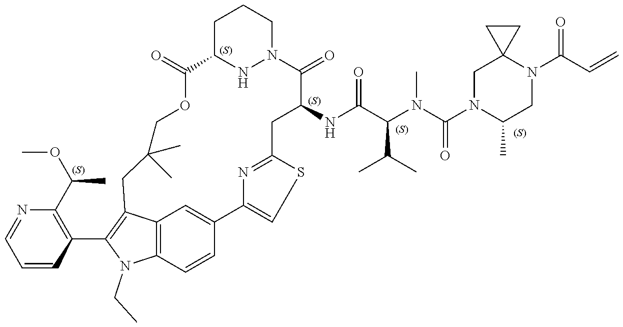 |
| A587 | 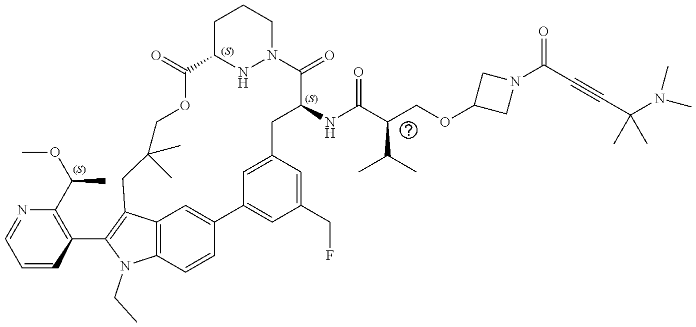 |
| A588 | 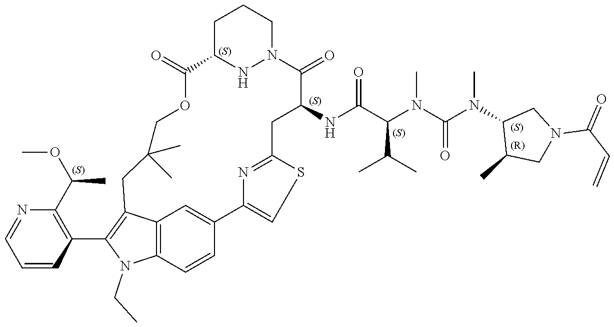 |
| A589 | 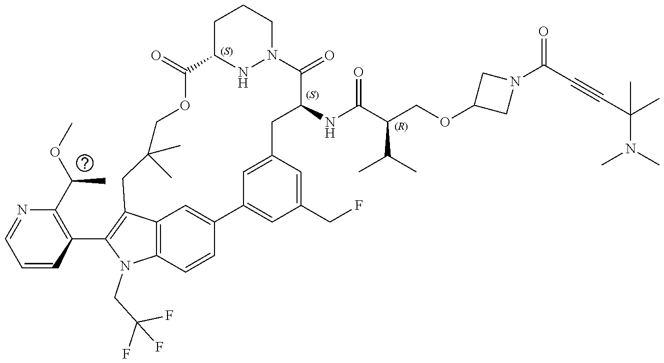 |
| A590 | 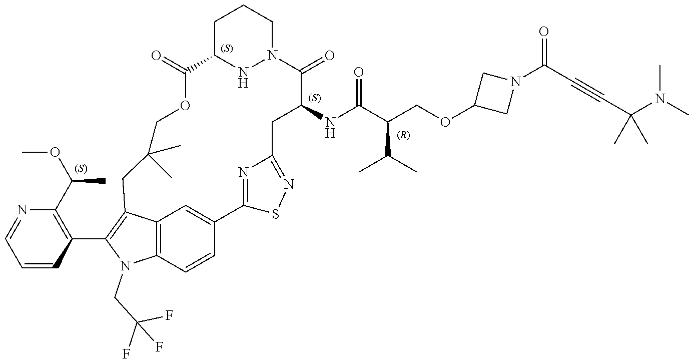 |
| A591 | 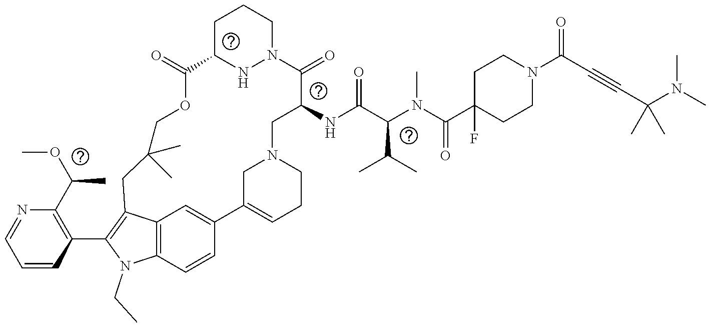 |
| A592 | 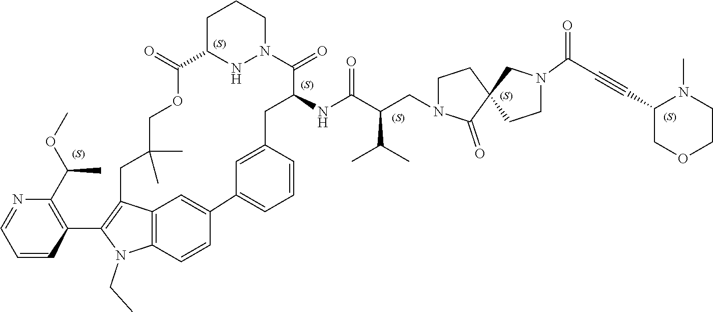 |
| A593 | 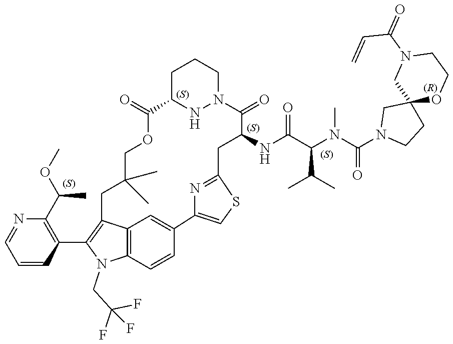 |
| A594 | 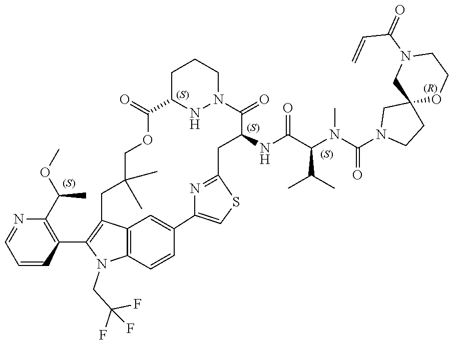 |
| A595 | 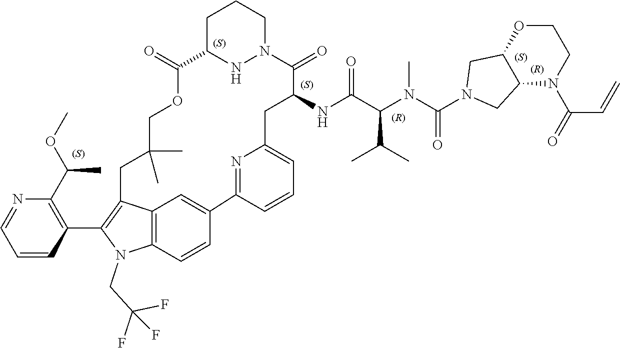 |
| A596 | 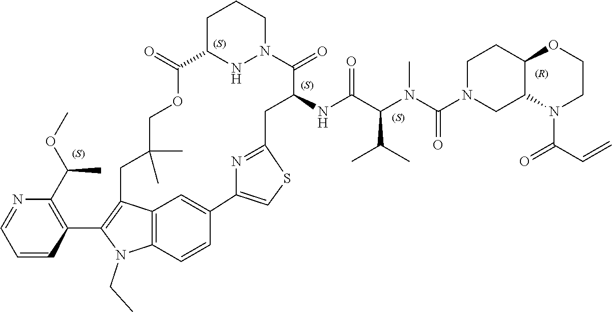 |
| A597 | 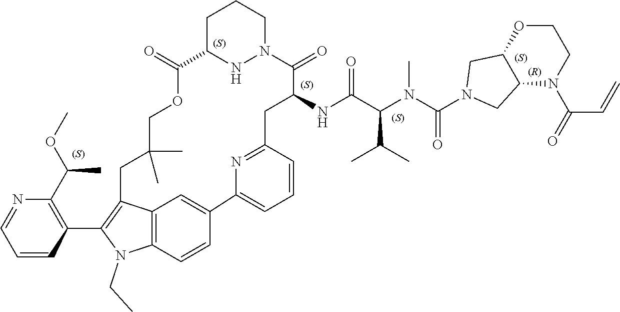 |
| A598 | 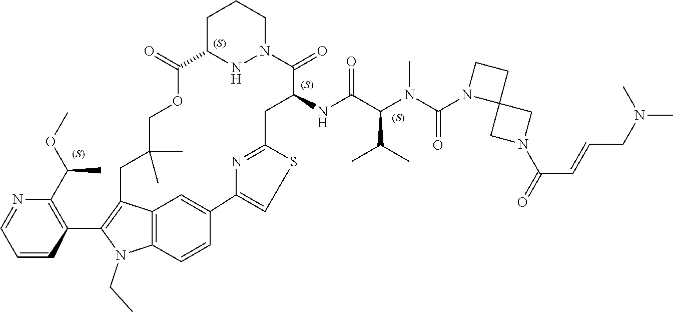 |
| A599 | 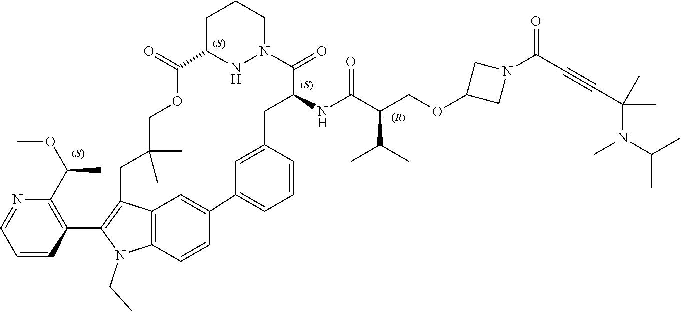 |
| A600 | 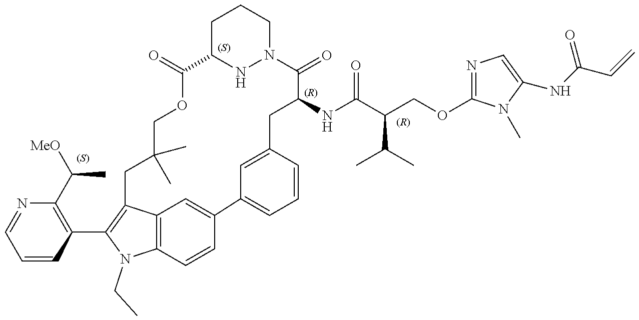 |
| A601 | 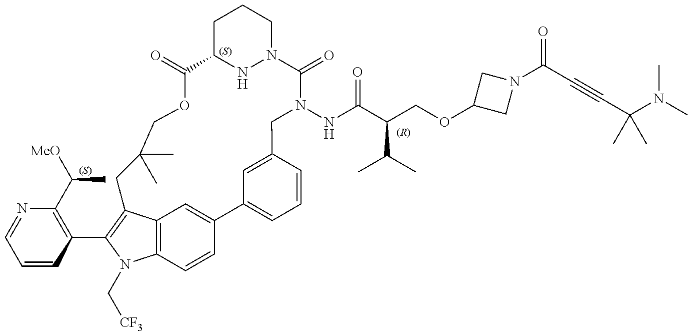 |
| A602 | 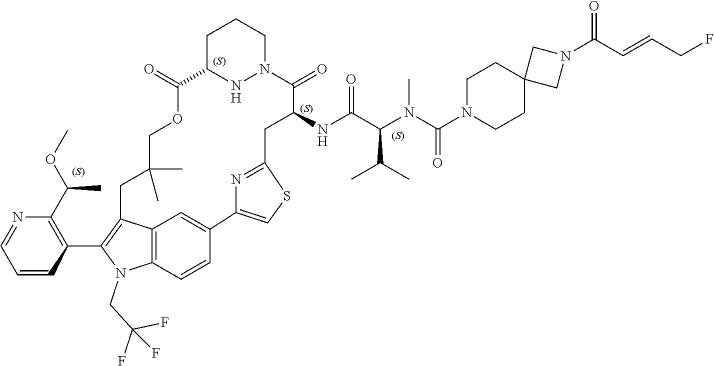 |
| A603 | 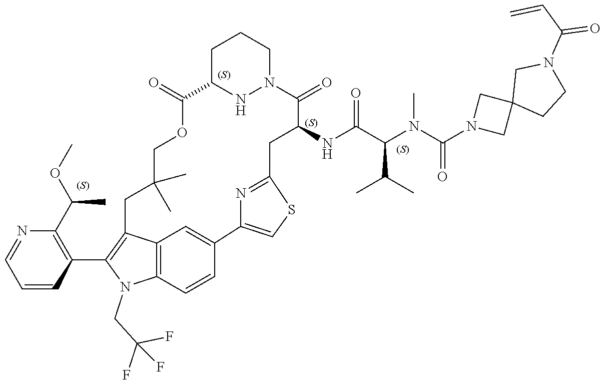 |
| A604 | 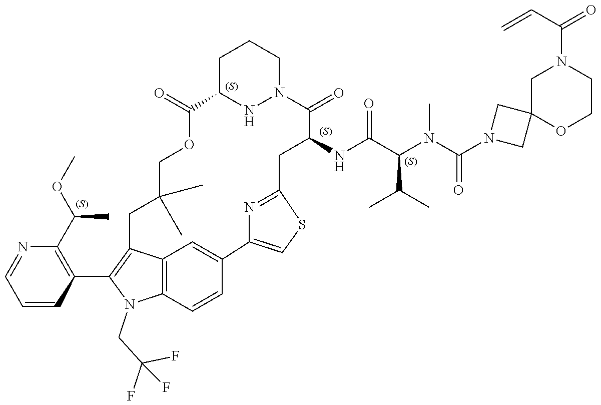 |
| A605 | 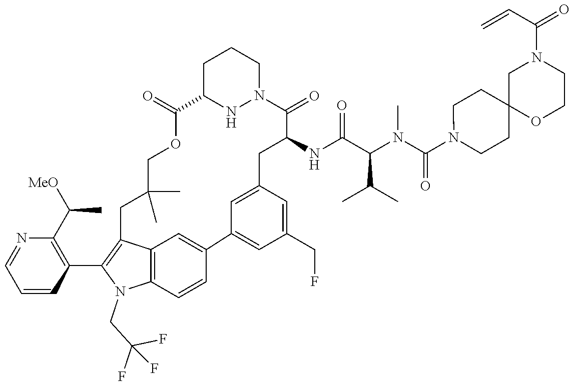 |
| A606 | 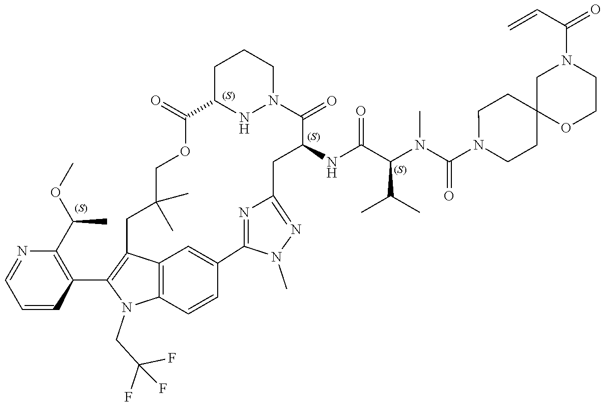 |
| A607 | 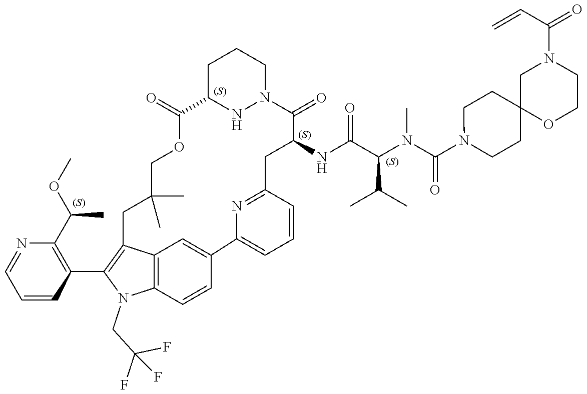 |
| A608 | 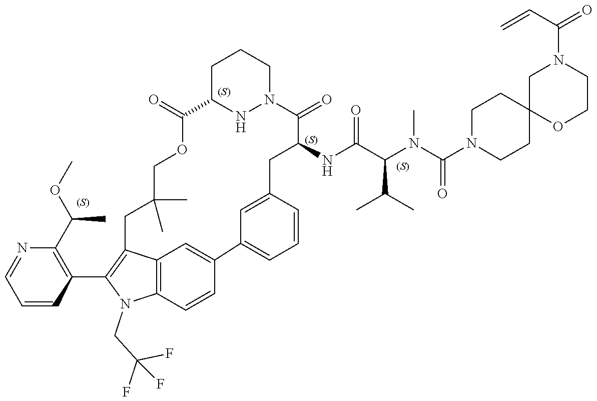 |
| A609 | 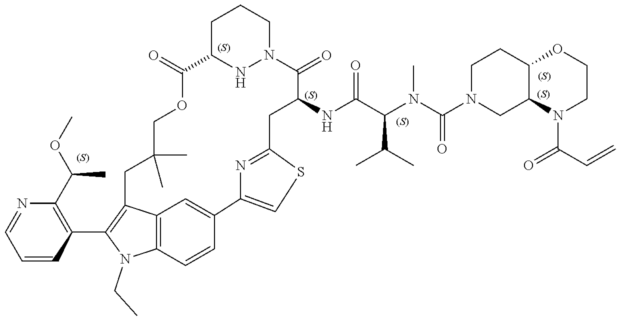 |
| A610 | 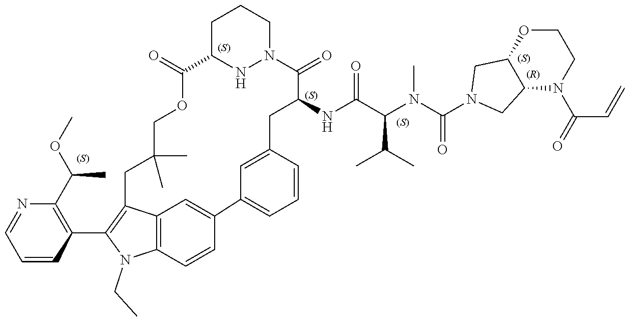 |
| A611 | 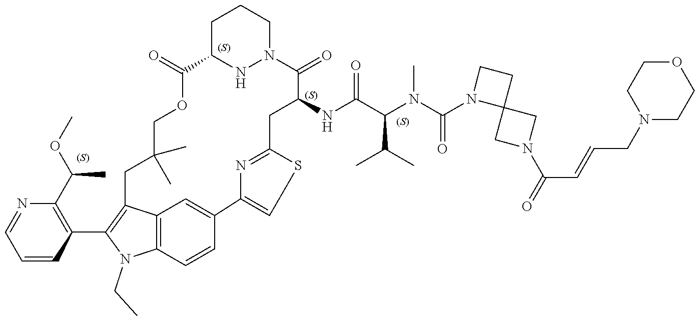 |
| A612 | 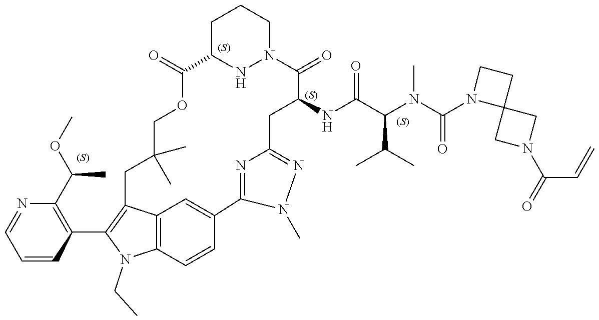 |
| A613 | 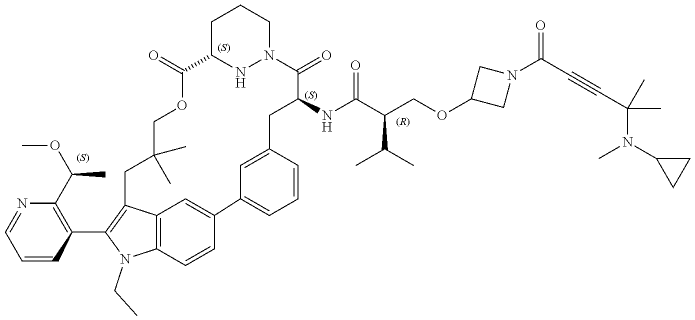 |
| A614 | 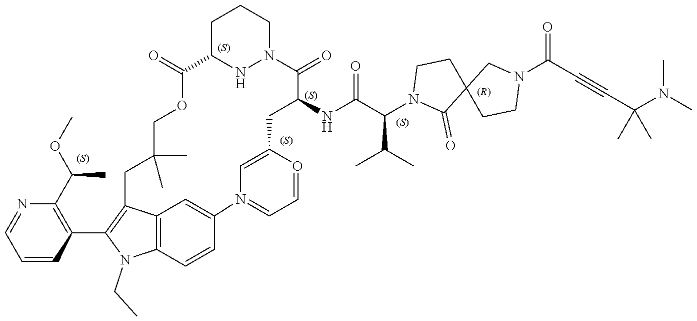 |
| A615 | 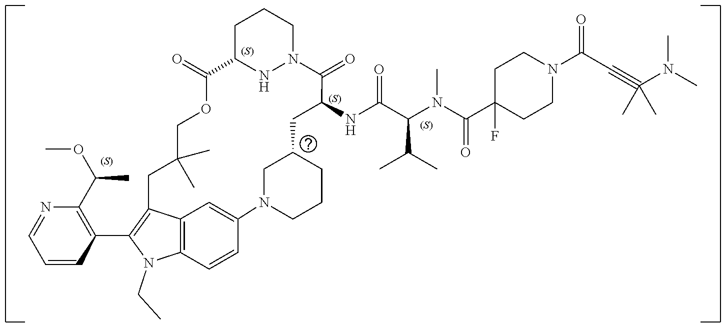 |
| A616 | 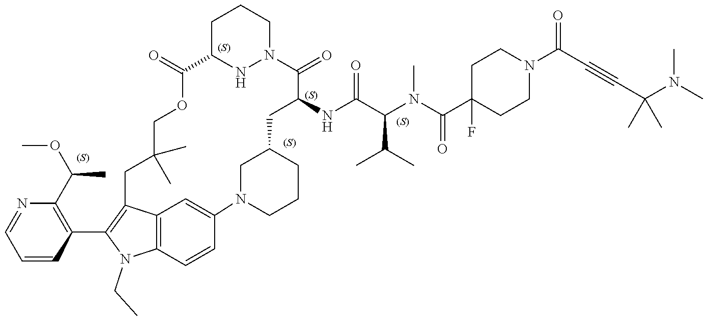 |
| A617 | 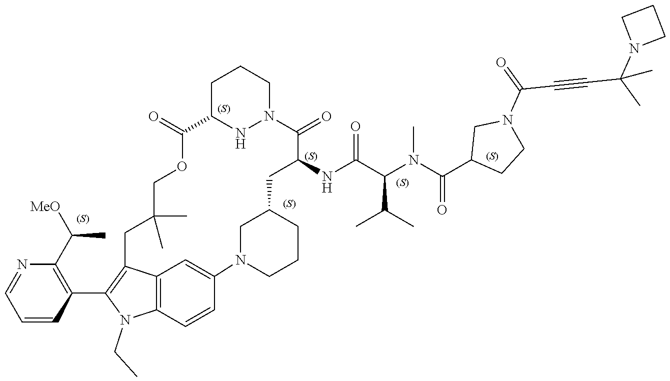 |
| A618 | 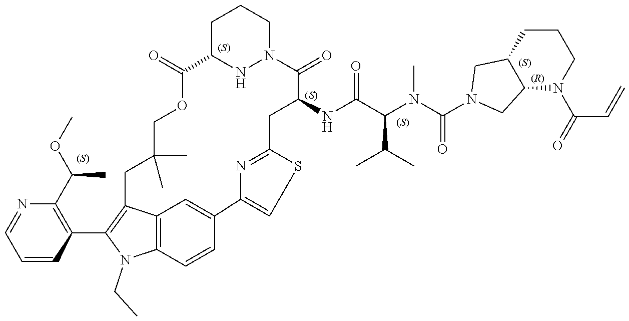 |
| A619 | 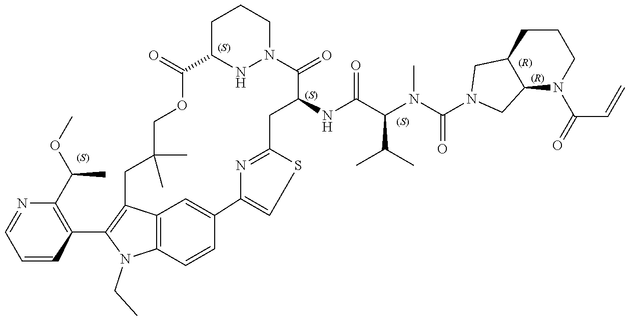 |
| A620 | 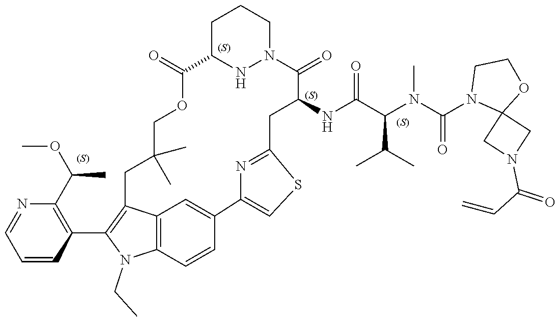 |
| A621 | 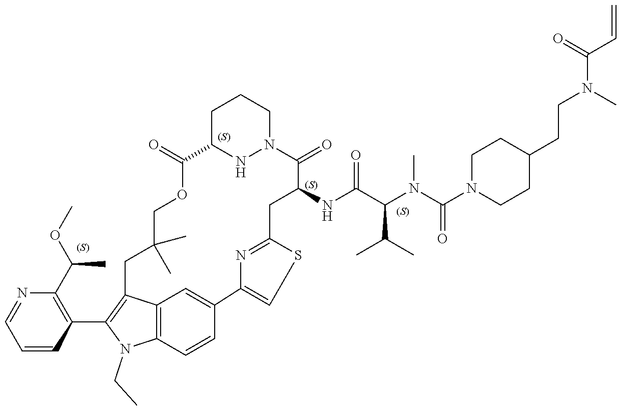 |
| A622 | 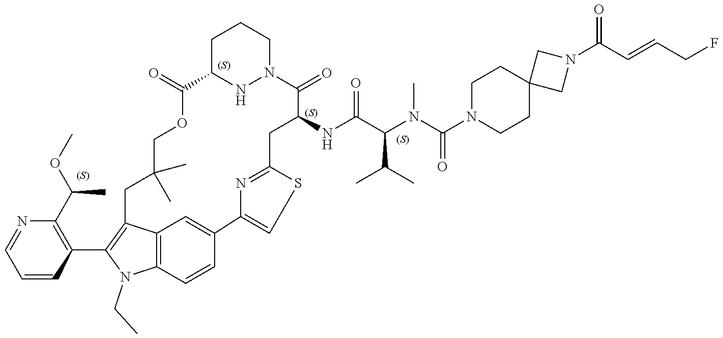 |
| A623 | 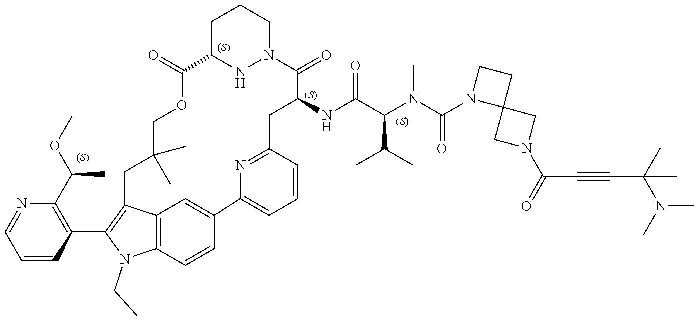 |
| A624 | 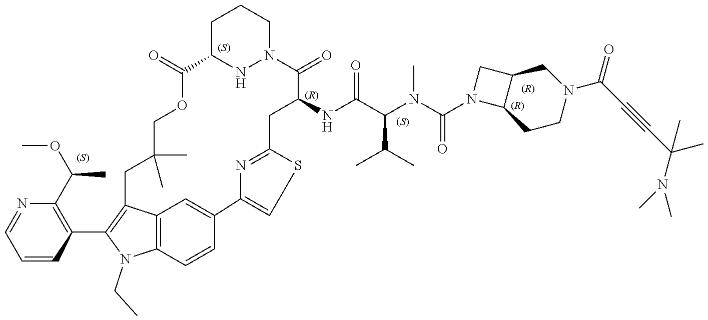 |
| A625 | 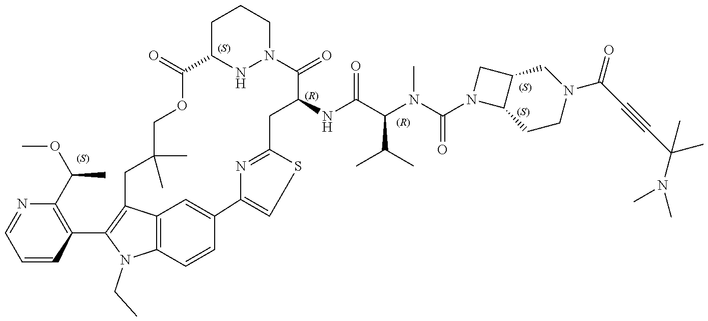 |
| A626 | 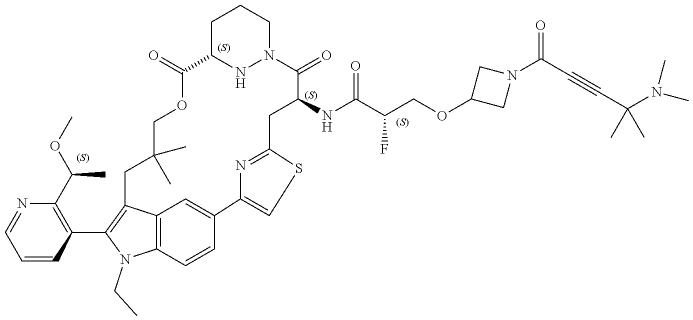 |
| A627 | 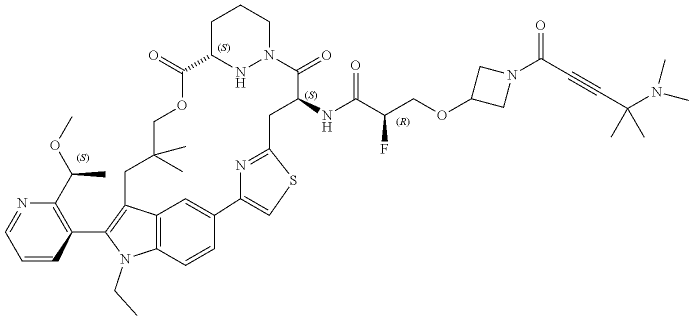 |
| A628 | 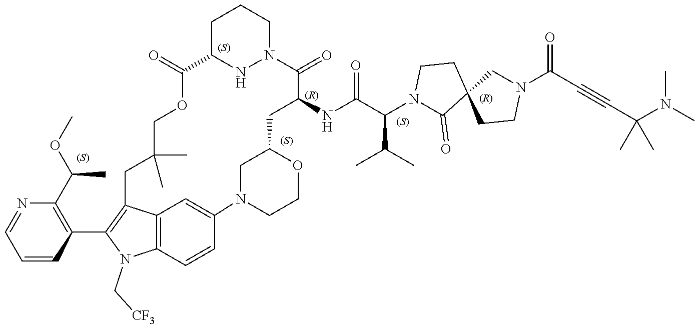 |
| A629 | 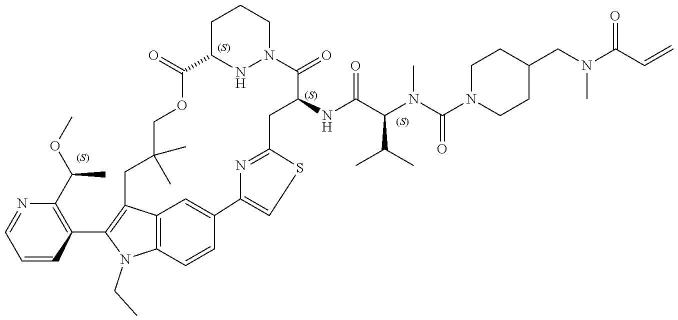 |
| A630 | 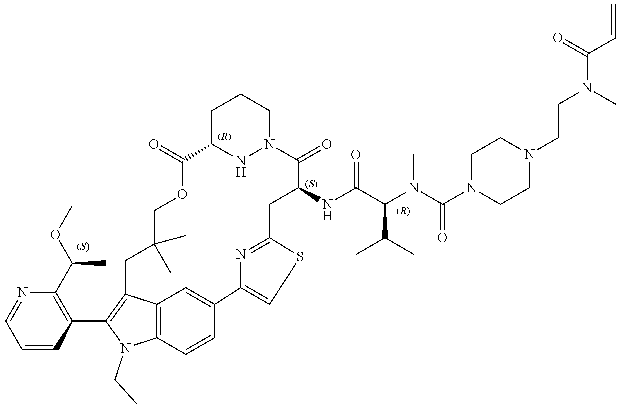 |
| A631 | 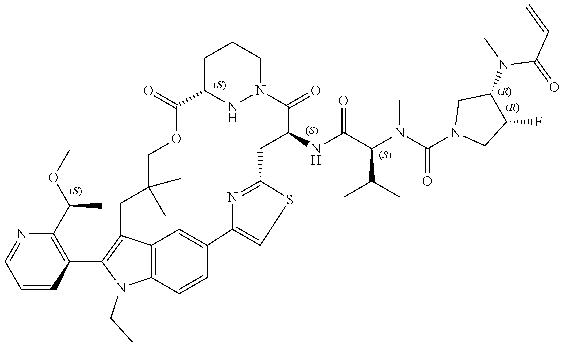 |
| A632 | 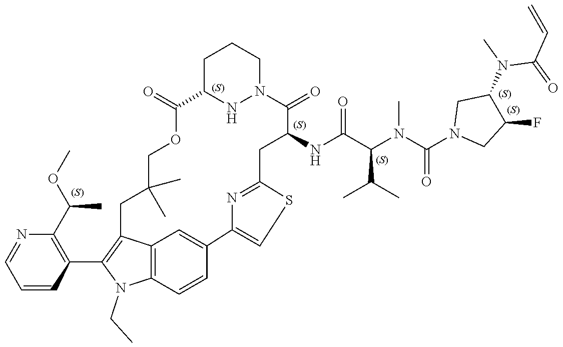 |
| A633 | 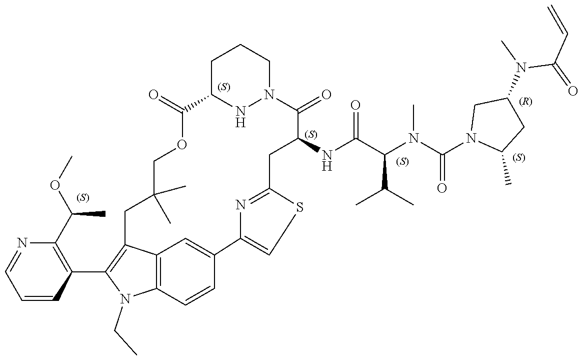 |
| A634 | 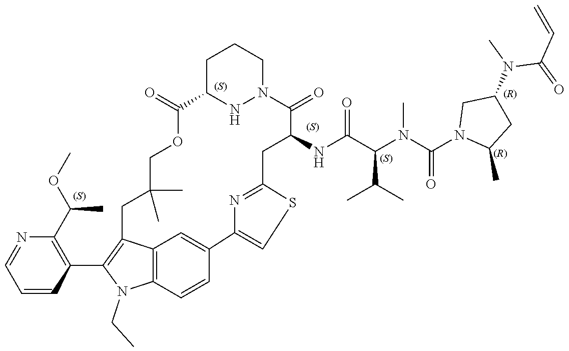 |
| A635 | 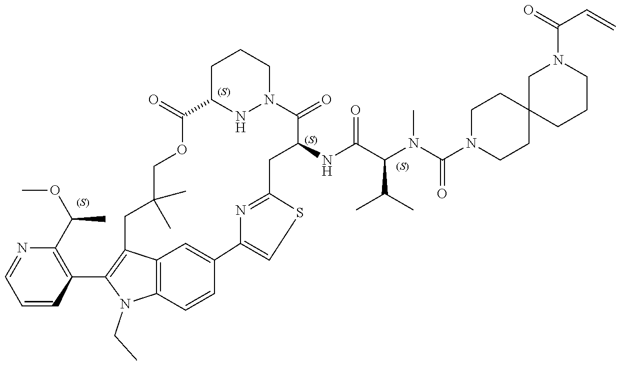 |
| A636 | 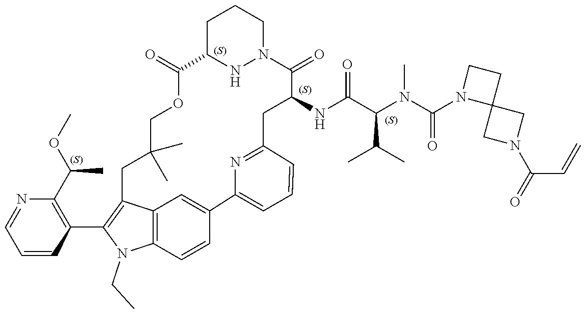 |
| A637 | 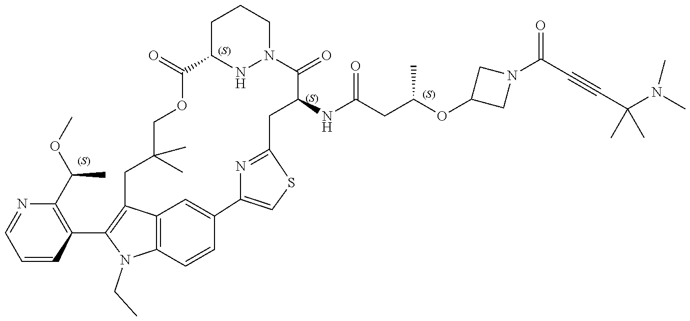 |
| A638 | 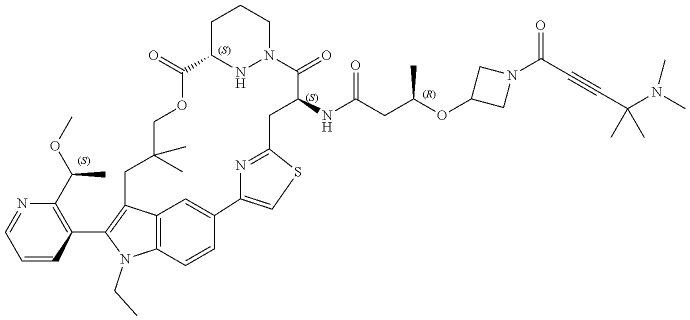 |
| A639 | 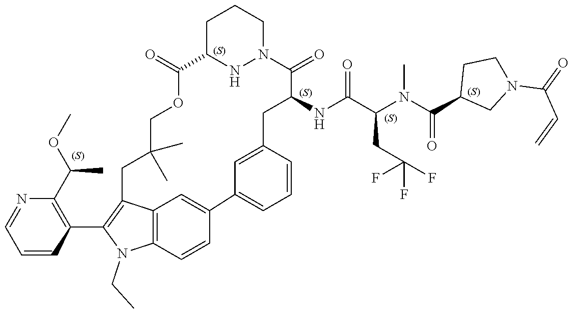 |
| A640 | 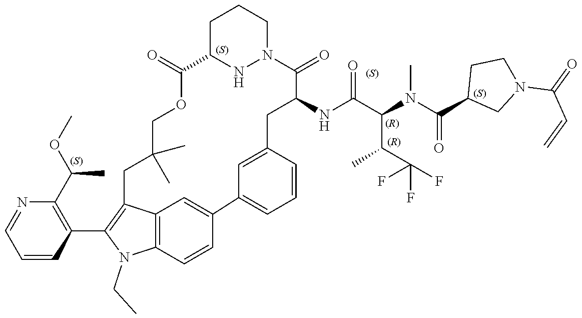 |
| A641 | 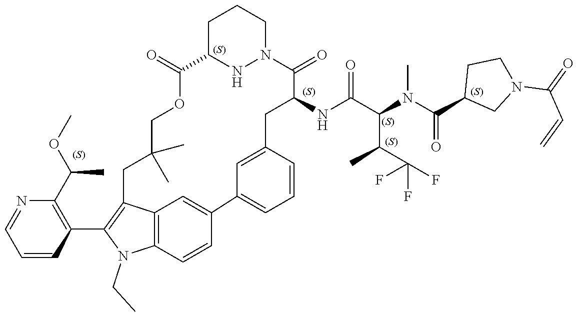 |
| A642 | 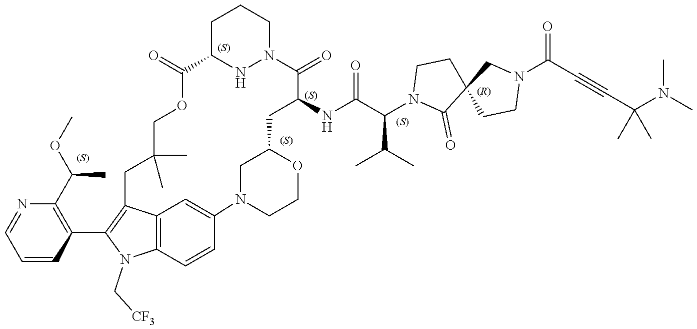 |
| A643 | 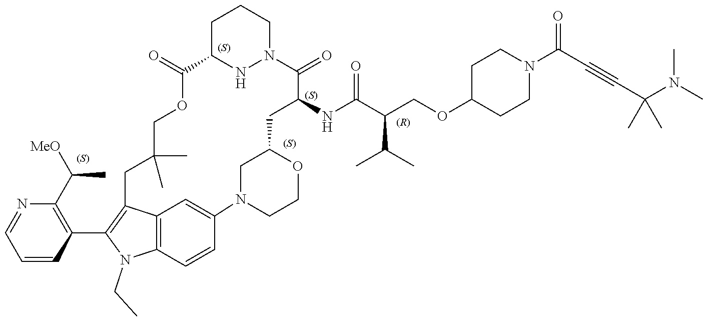 |
| A644 | 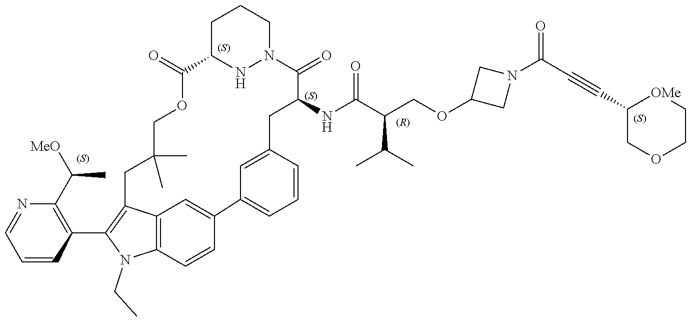 |
| A645 | 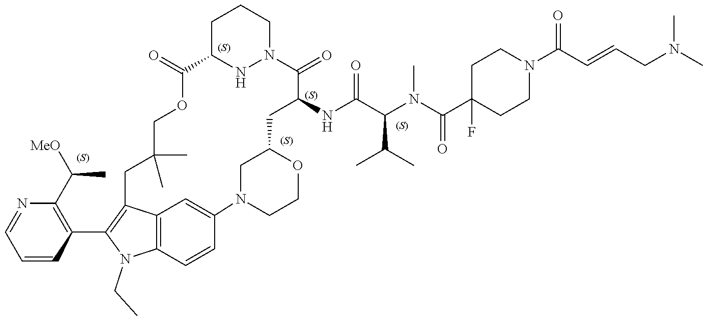 |
| A646 | 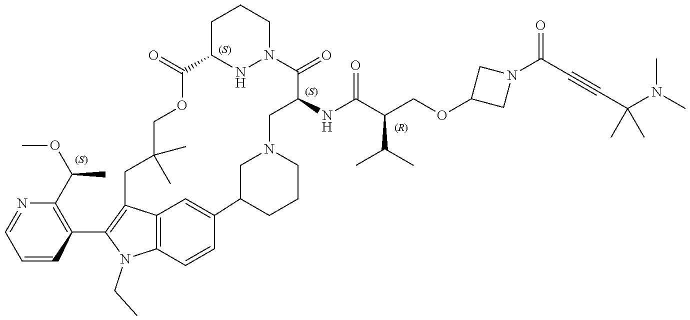 |
| A647 | 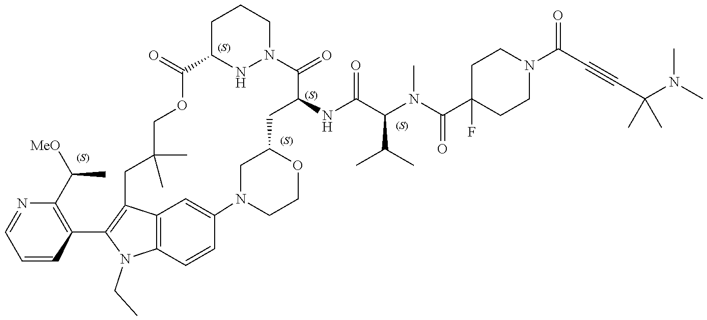 |
| A648 | 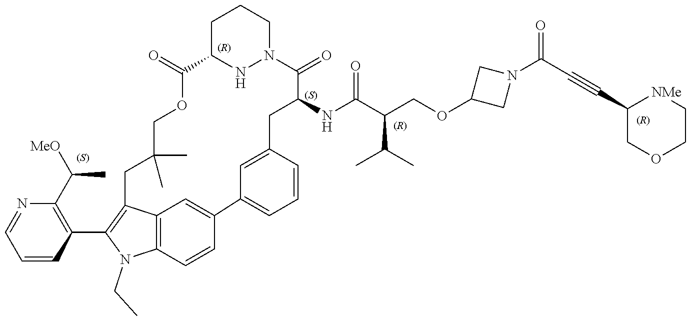 |
| A649 | 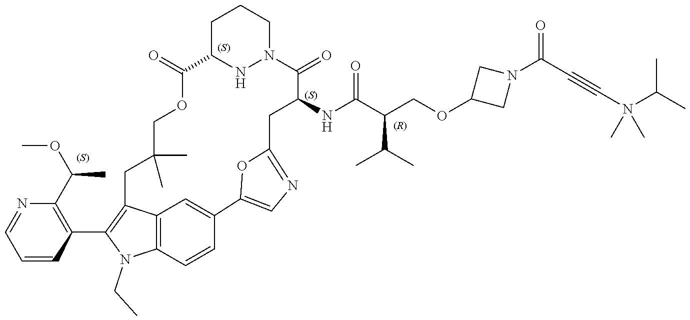 |
| A650 | 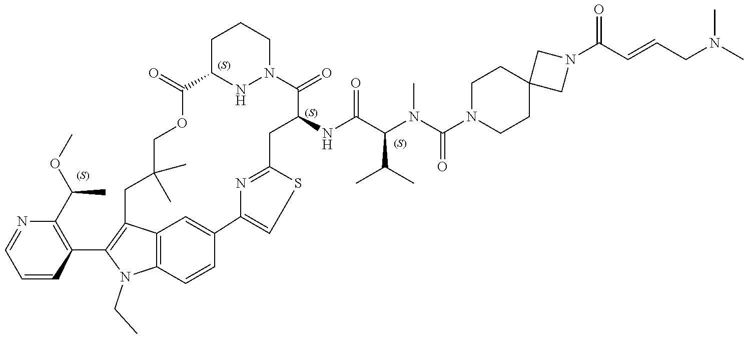 |
| A651 | 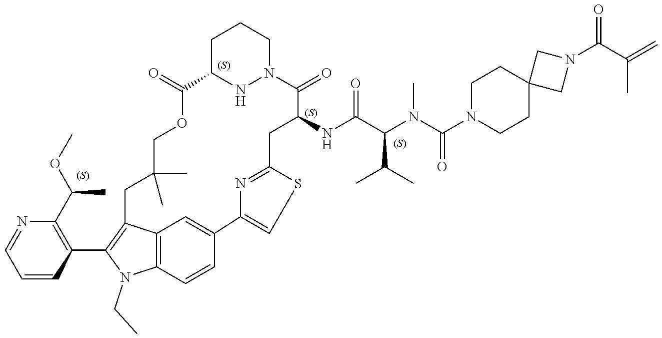 |
| A652 | 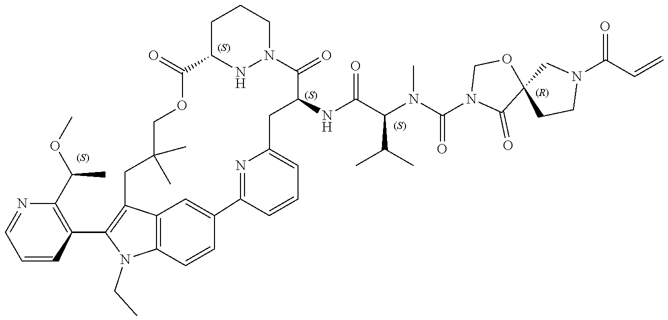 |
| A653 | 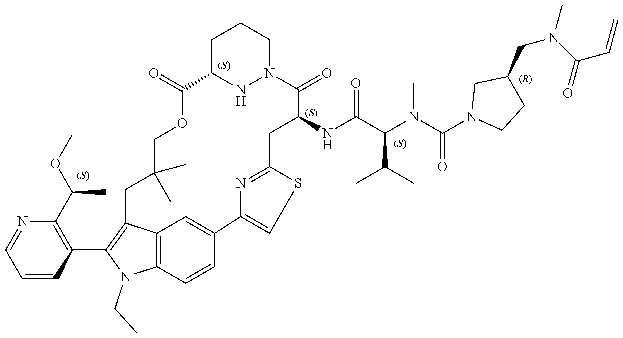 |
| A654 | 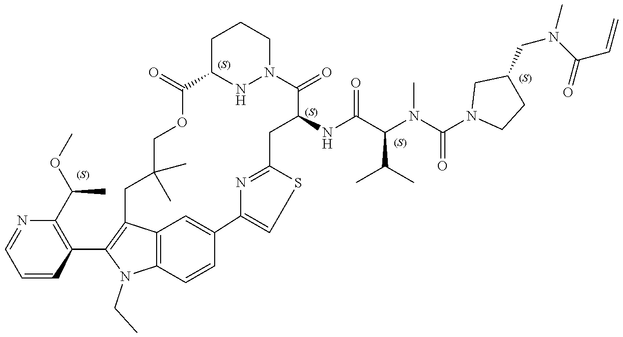 |
| A655 | 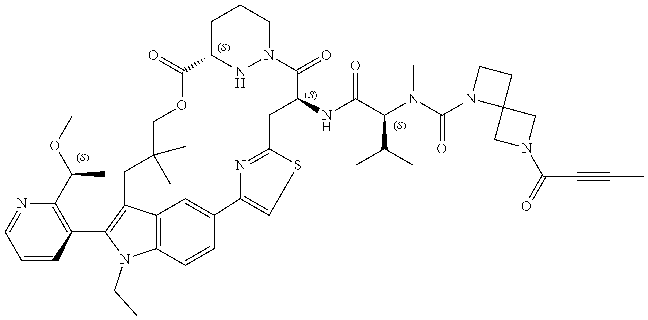 |
| A656 | 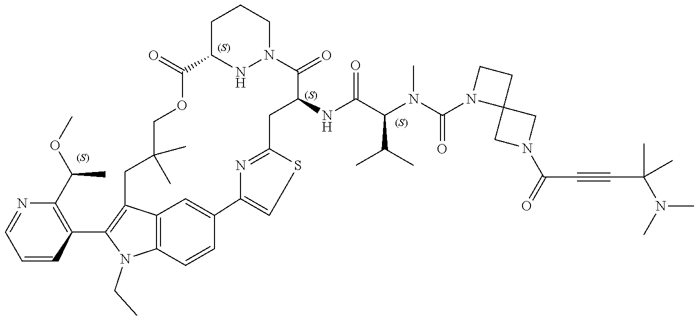 |
| A657 | 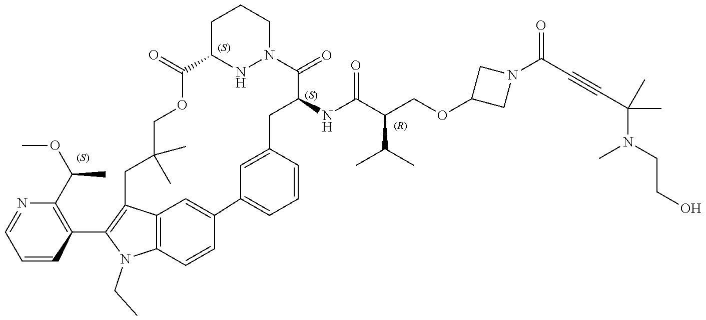 |
| A658 | 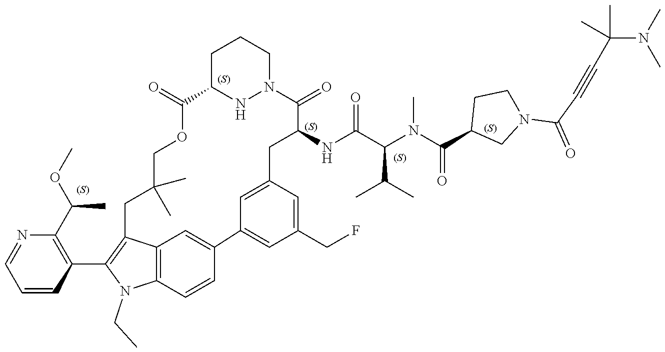 |
| A659 | 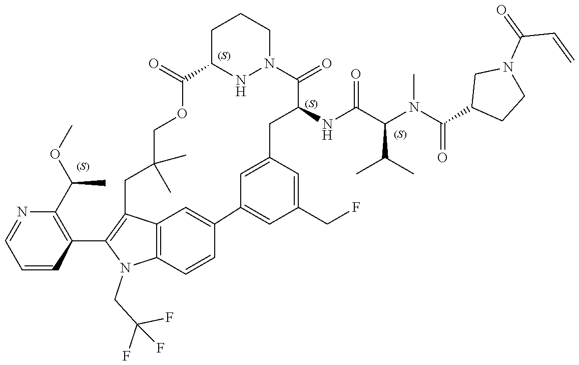 |
| A660 | 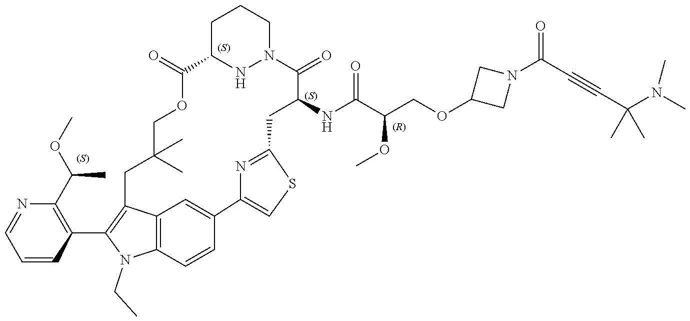 |
| A661 | 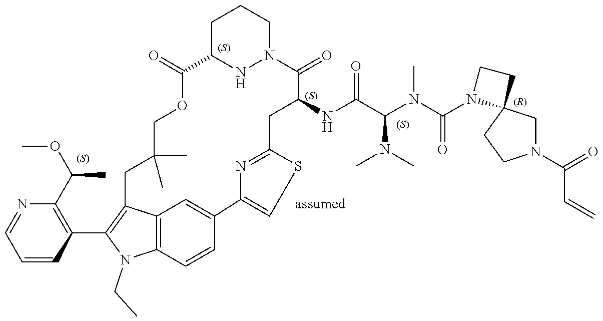 |
| A662 | 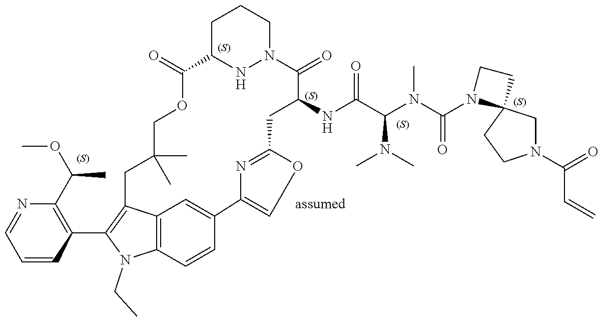 |
| A663 | 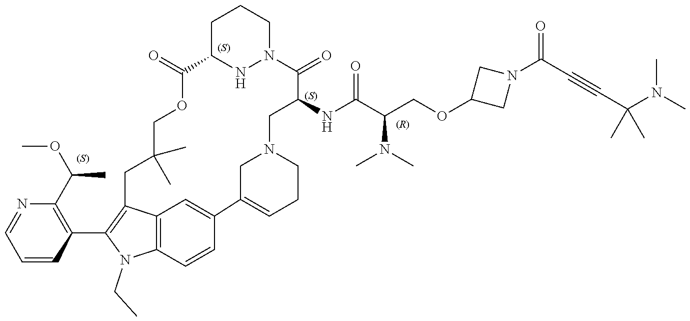 |
| A664 | 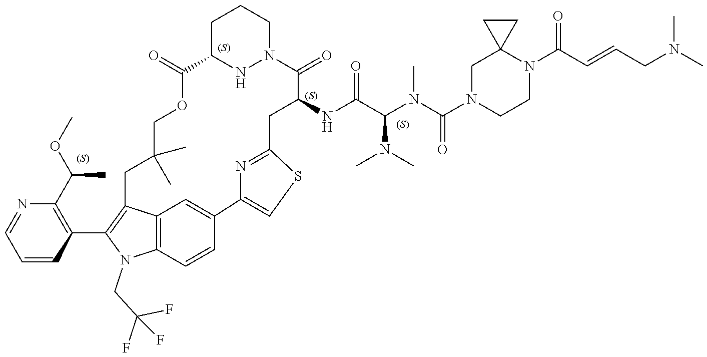 |
| A665 | 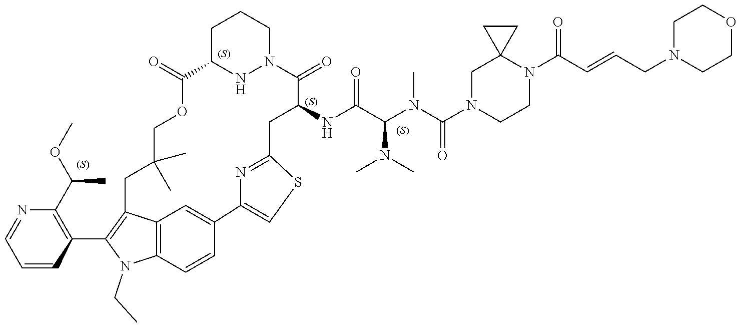 |
| A666 | 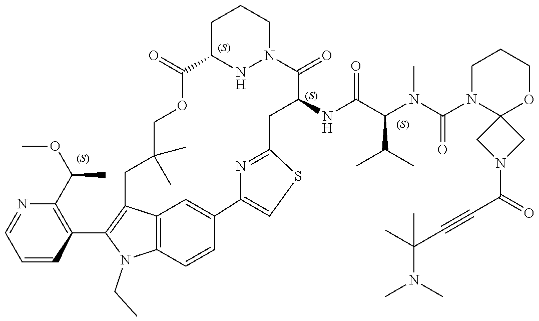 |
| A667 | 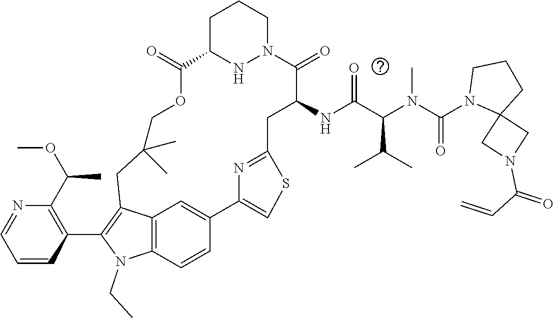 |
| A668 | 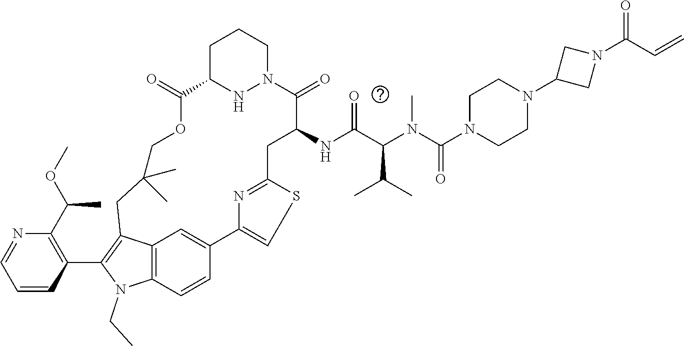 |
| A669 | 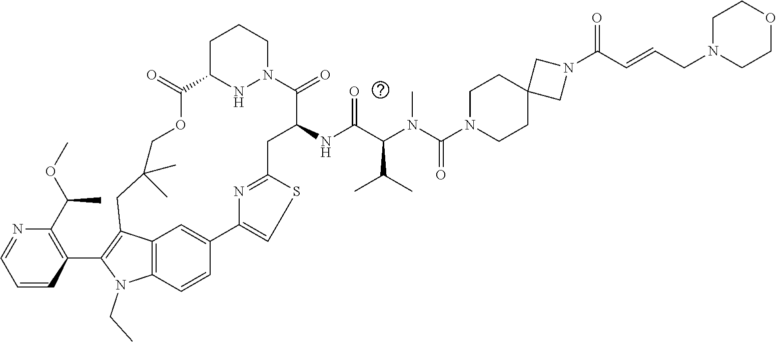 |
| A670 | 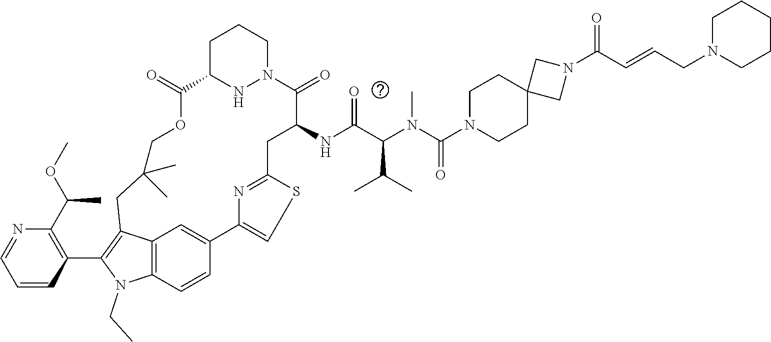 |
| A671 | 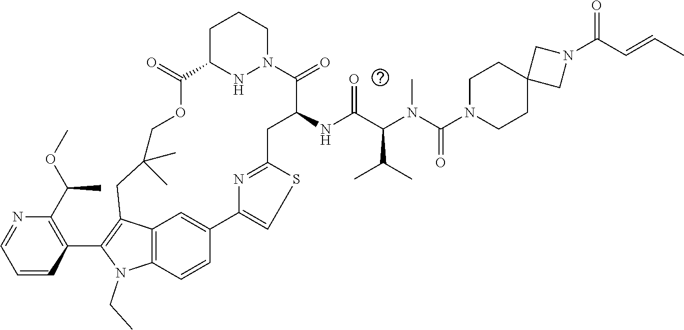 |
| A672 | 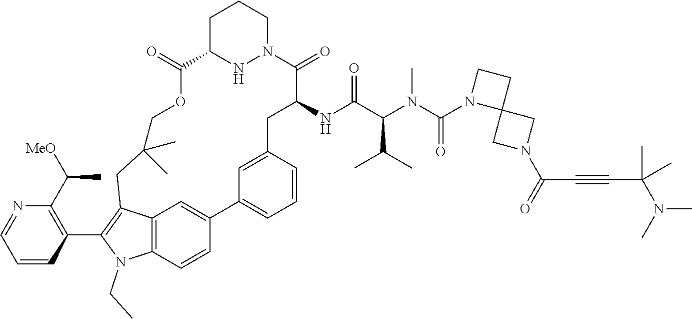 |
| A673 | 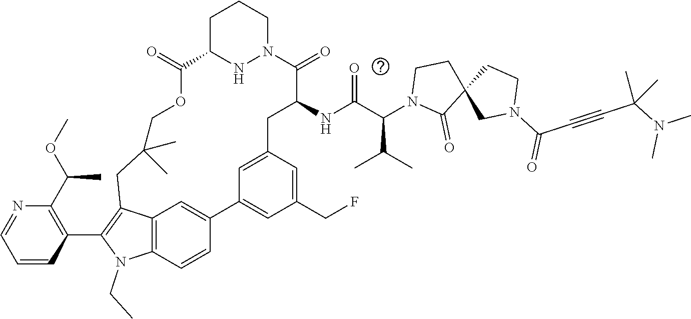 |
| A674 | 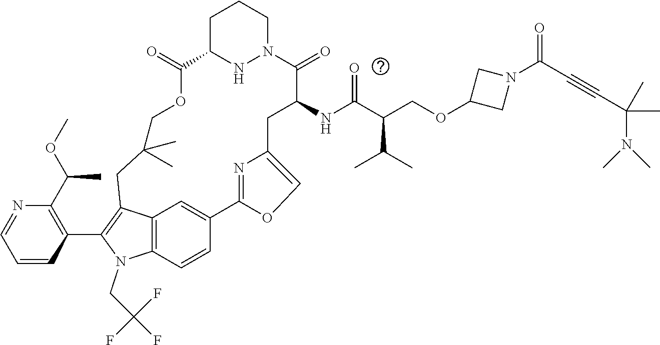 |
| A675 | 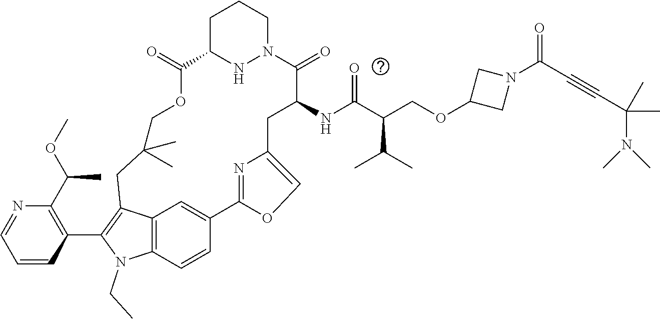 |
| A676 | 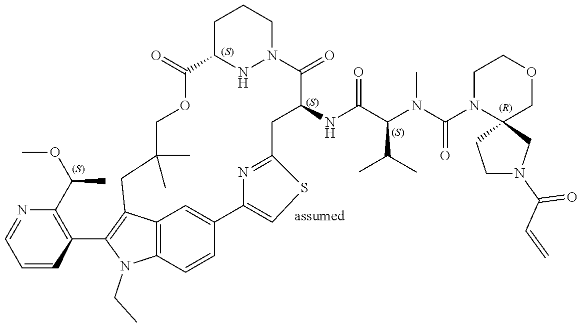 |
| A677 | 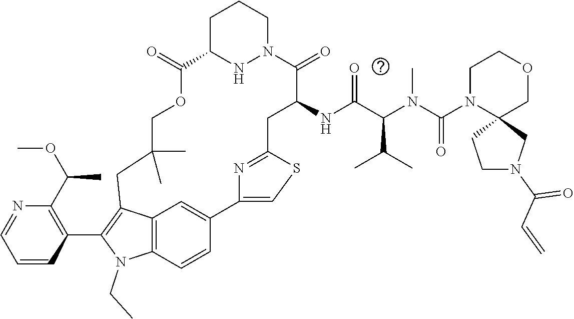 |
| A678 | 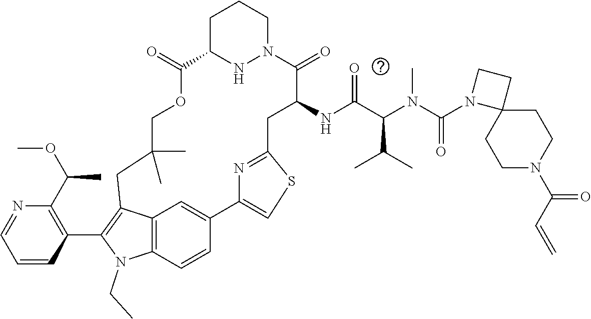 |
| A679 | 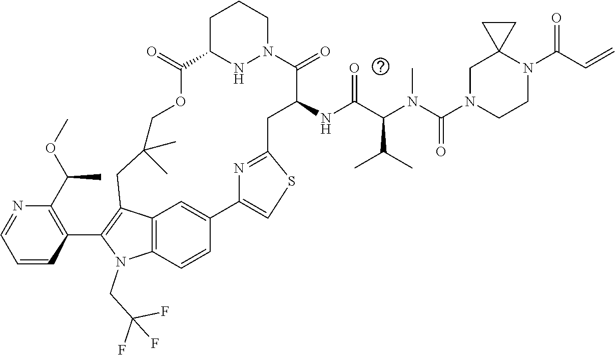 |
| A680 | 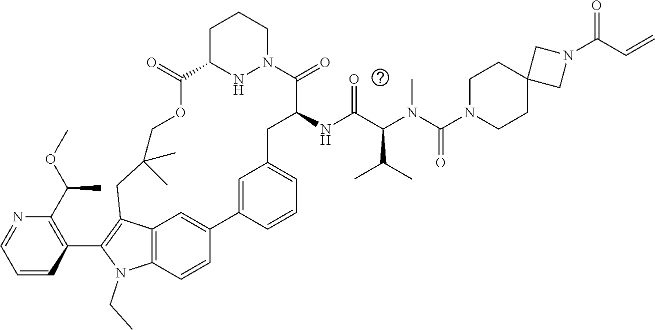 |
| A681 | 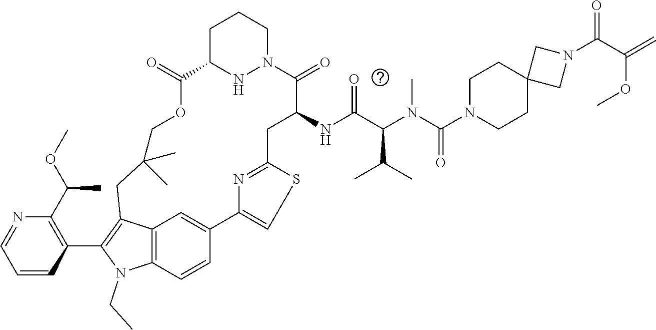 |
| A682 | 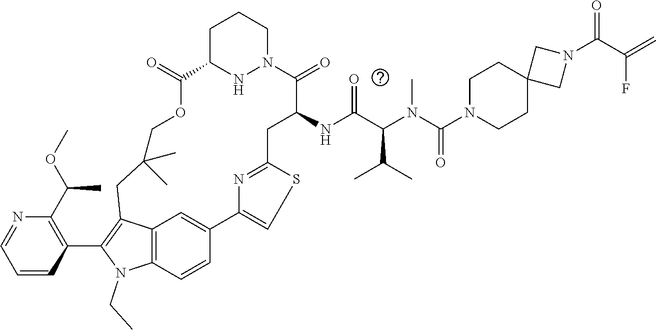 |
| A683 | 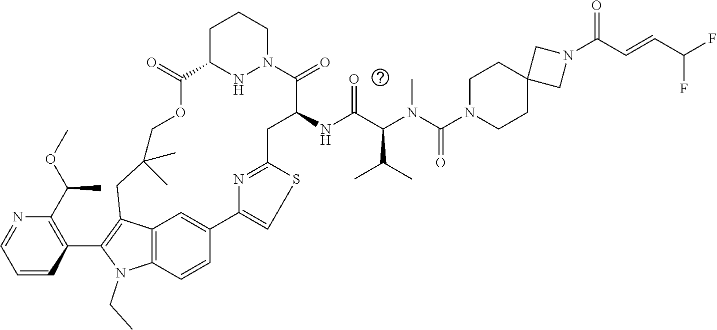 |
| A684 | 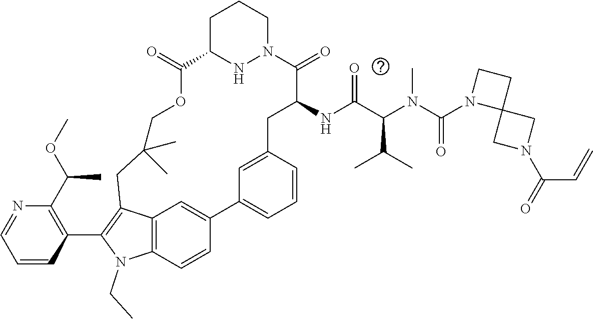 |
| A685 | 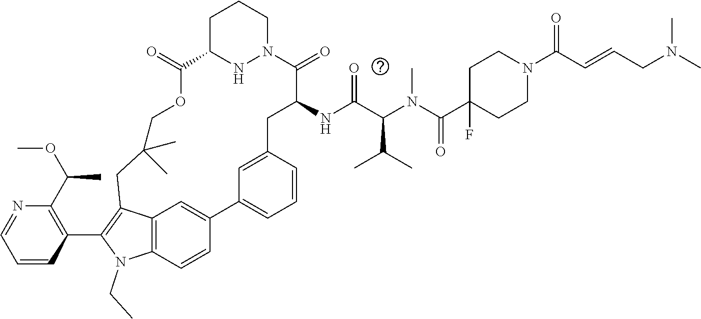 |
| A686 | 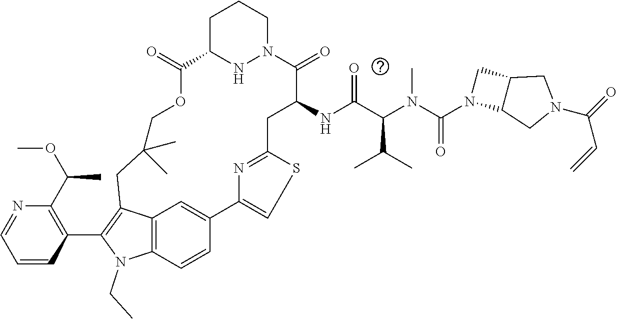 |
| A687 | 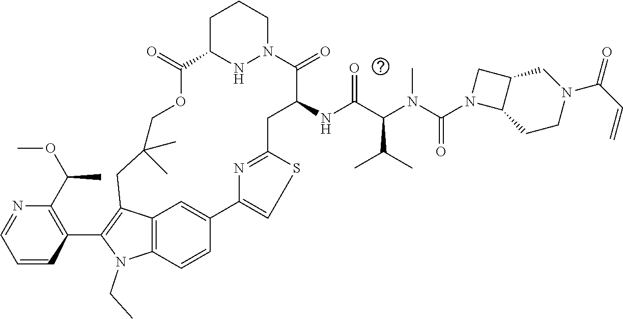 |
| A688 | 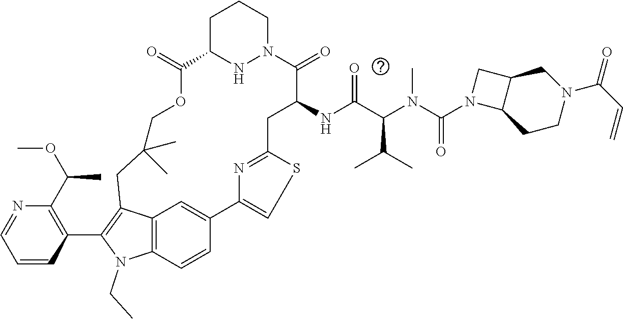 |
| A689 | 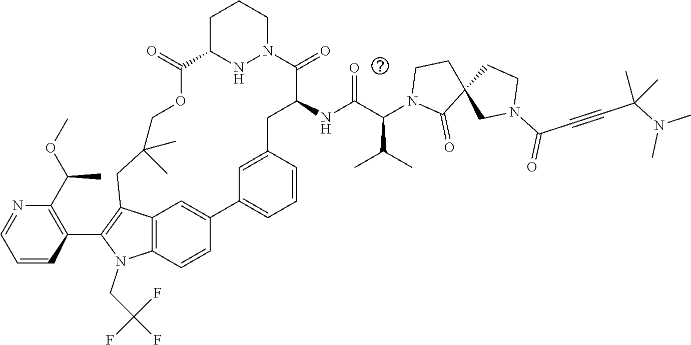 |
| A690 | 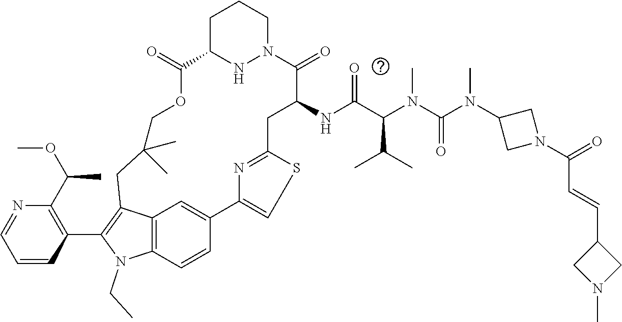 |
| A691 | 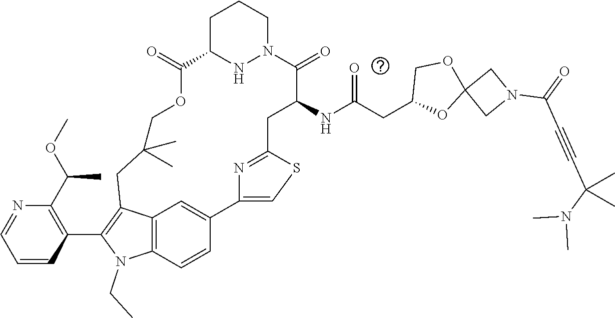 |
| A692 | 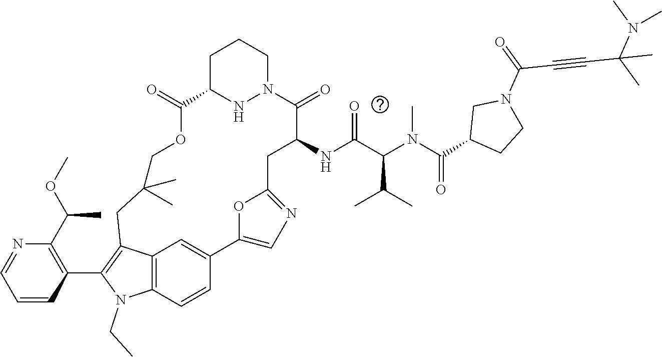 |
| A693 | 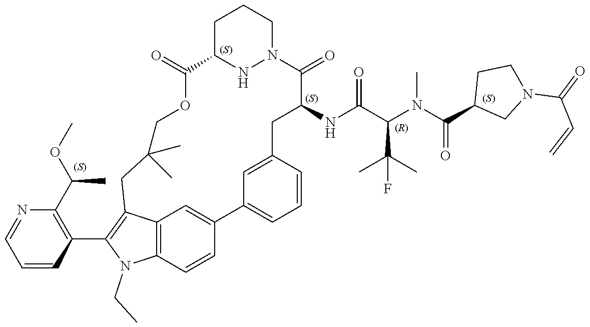 |
| A694 | 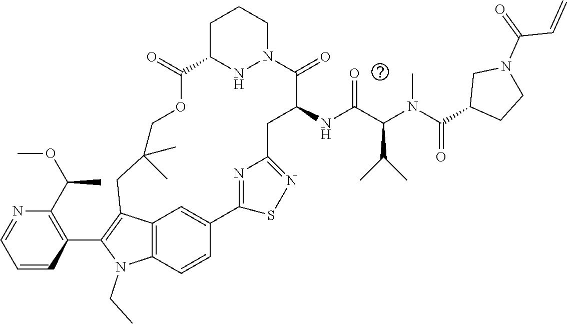 |
| A695 | 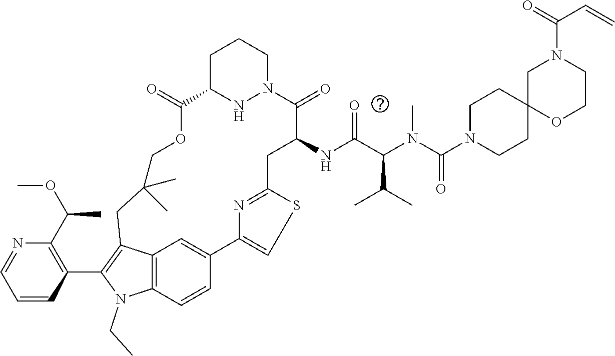 |
| A696 | 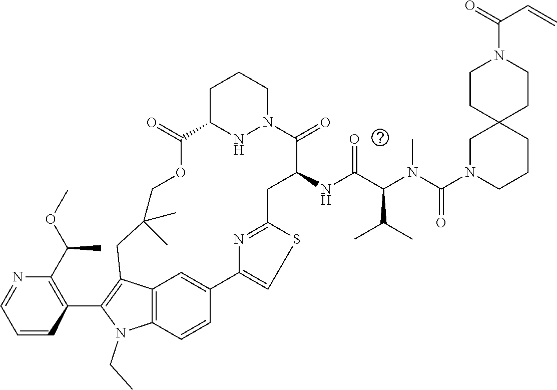 |
| A697 | 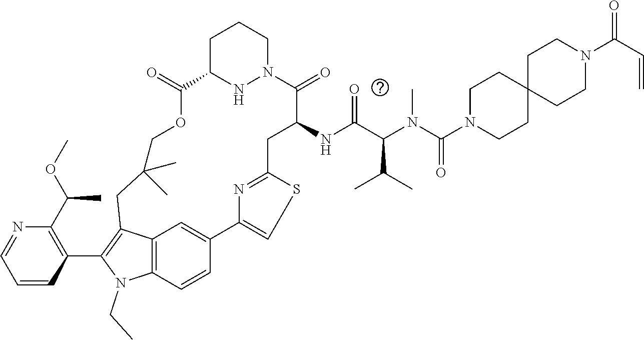 |
| A698 | 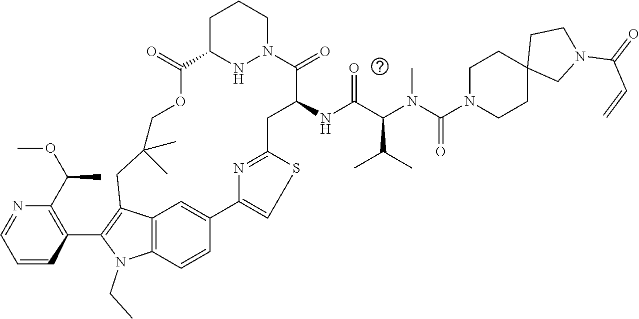 |
| A699 | 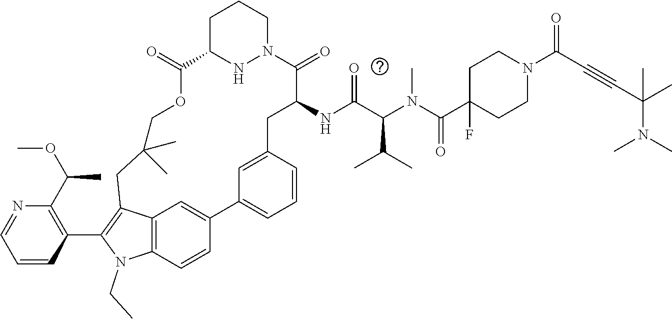 |
| A700 | 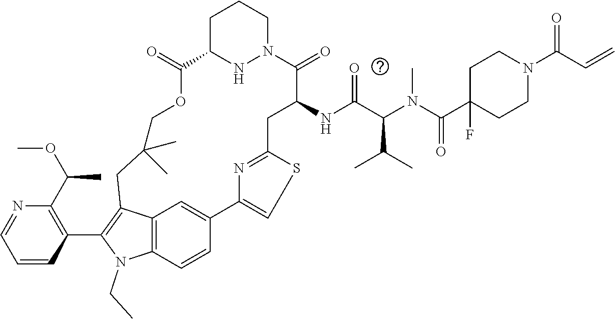 |
| A701 | 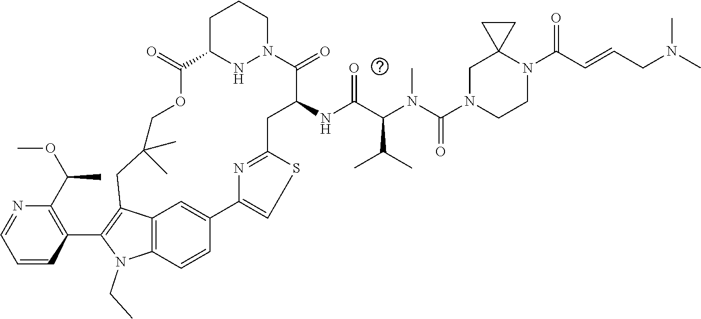 |
| A702 | 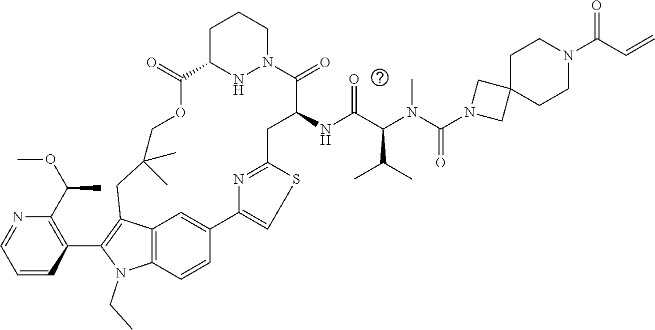 |
| A703 | 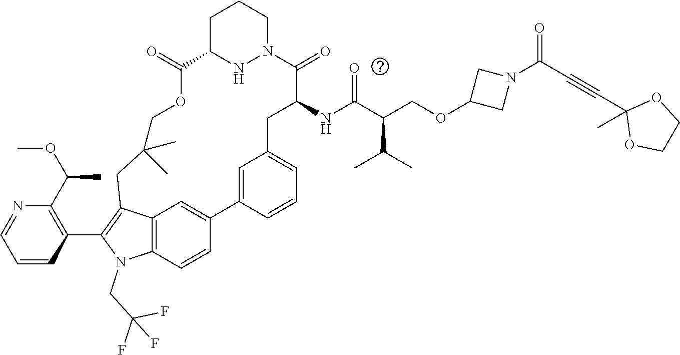 |
| A704 | 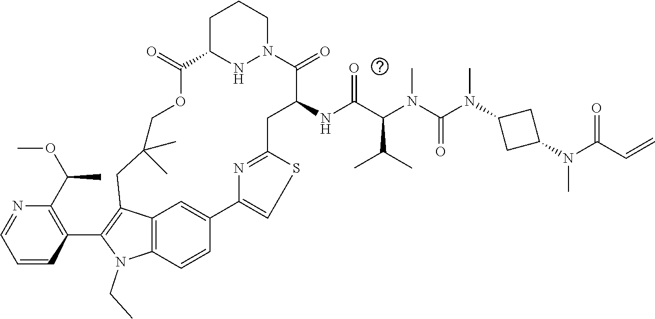 |
| A705 | 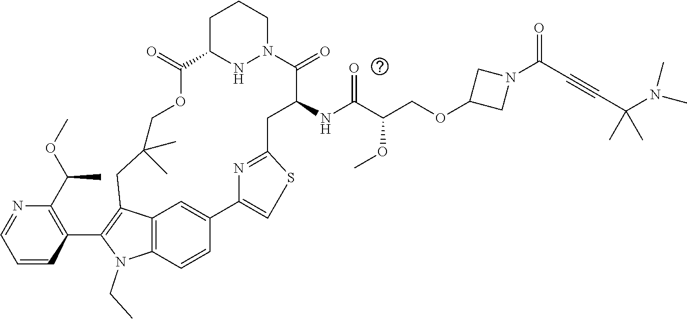 |
| A706 | 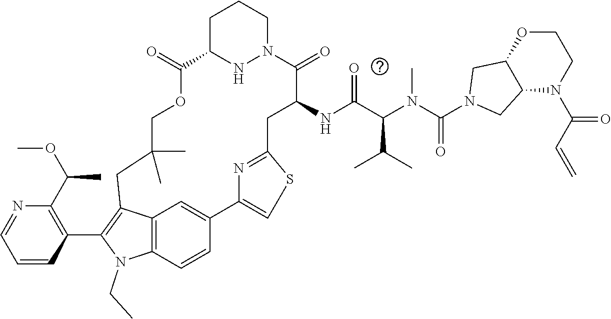 |
| A707 | 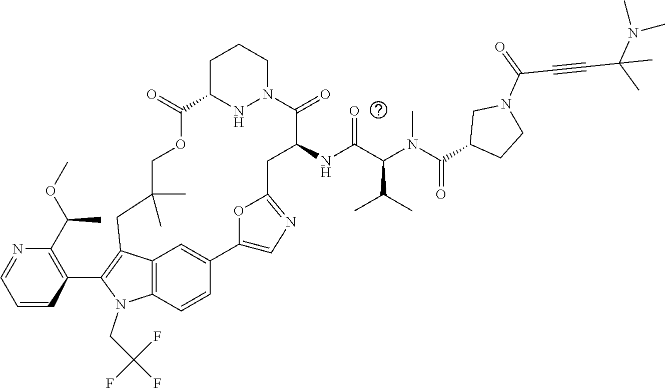 |
| A708 | 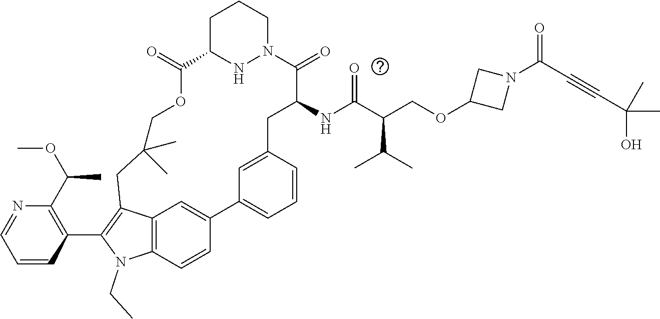 |
| A709 | 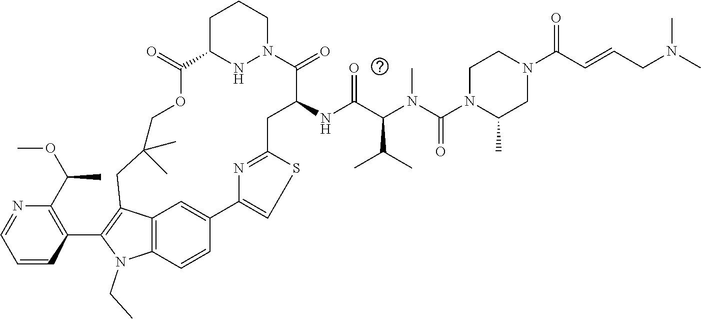 |
| A710 | 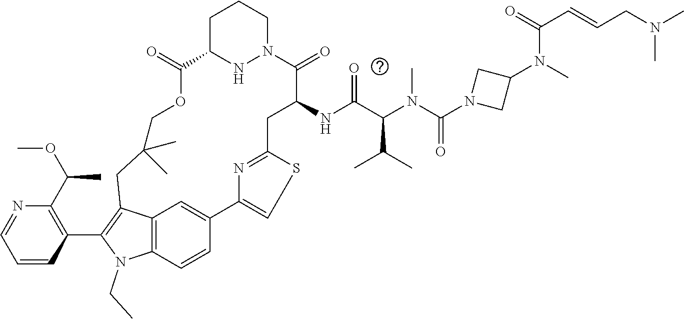 |
| A711 | 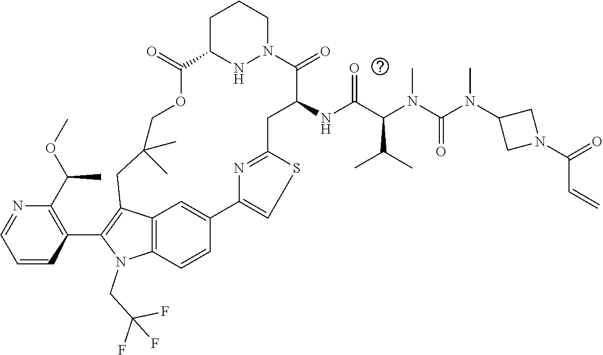 |
| A712 | 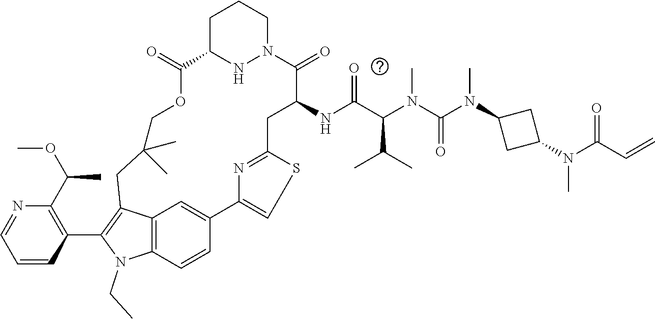 |
| A713 | 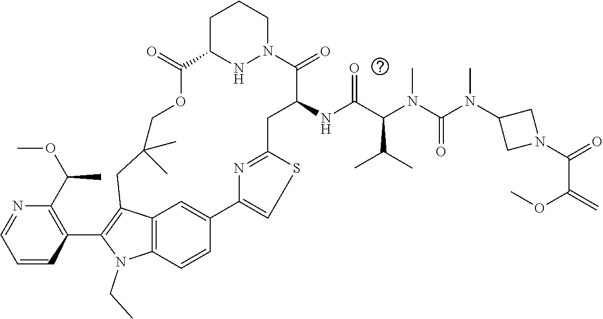 |
| A714 | 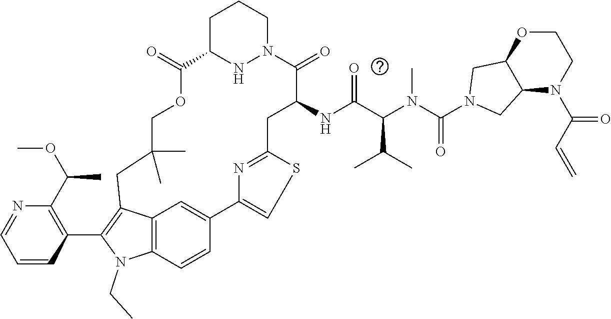 |
| A715 | 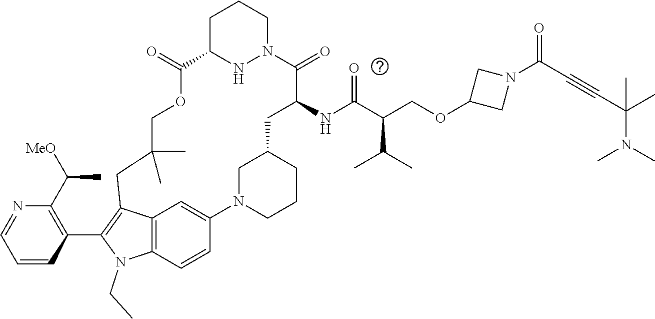 |
| A716 | 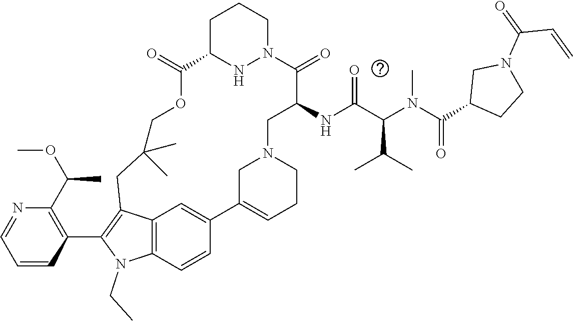 |
| A717 | 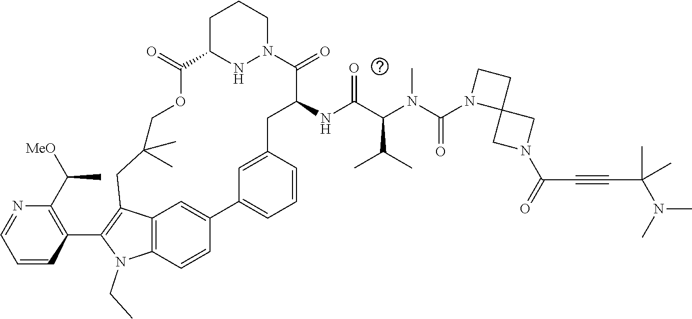 |
| A718 | 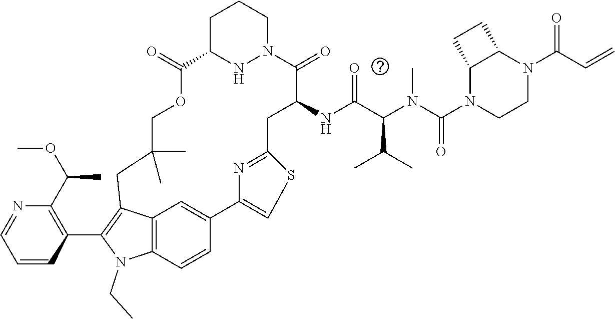 |
| A719 | 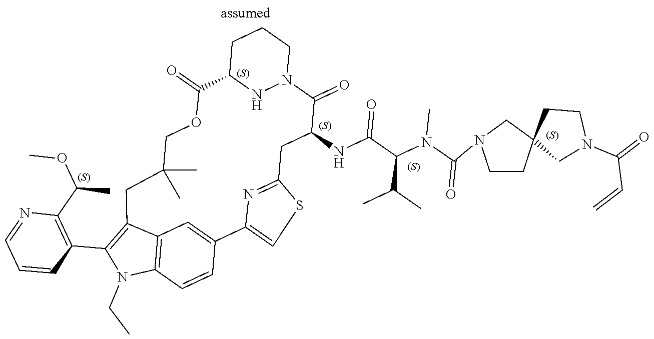 |
| A720 | 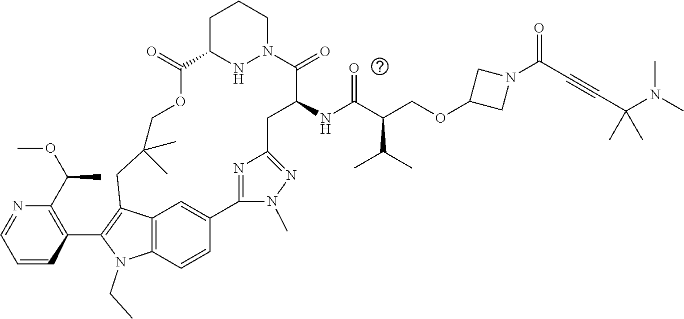 |
| A721 | 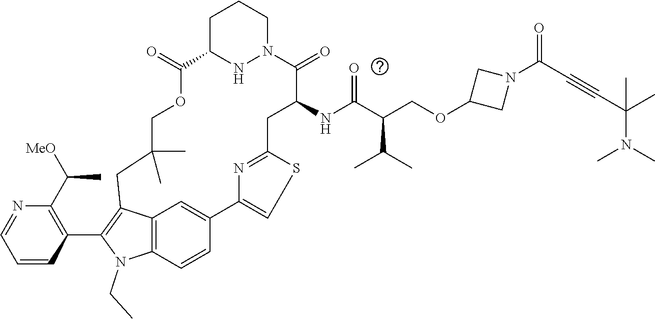 |
| A722 | 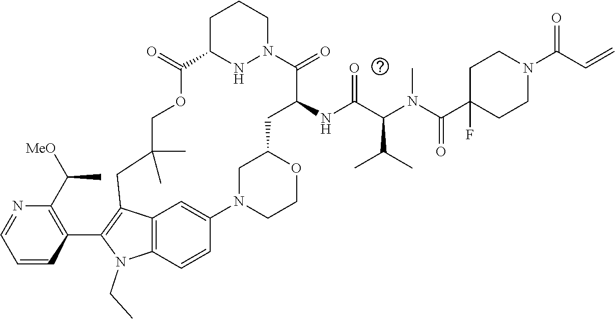 |
| A723 | 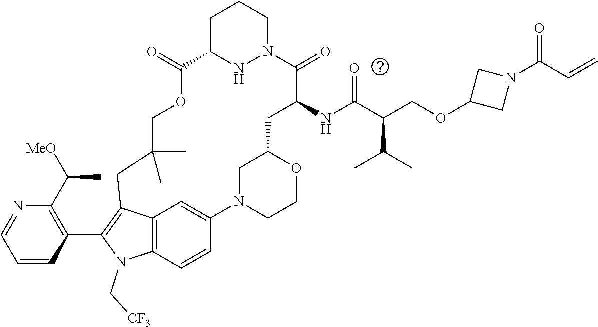 |
| A724 | 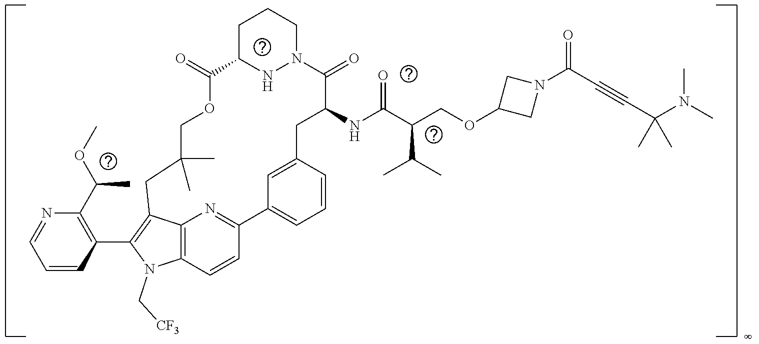 |
| A725 | 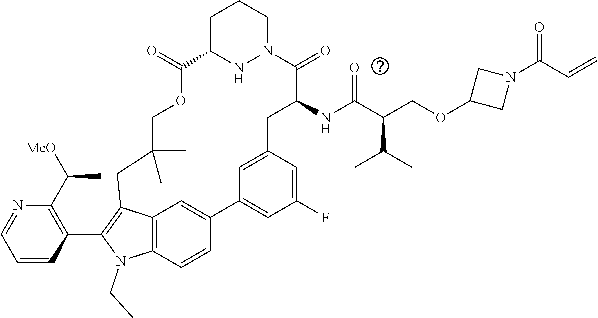 |
| A726 | 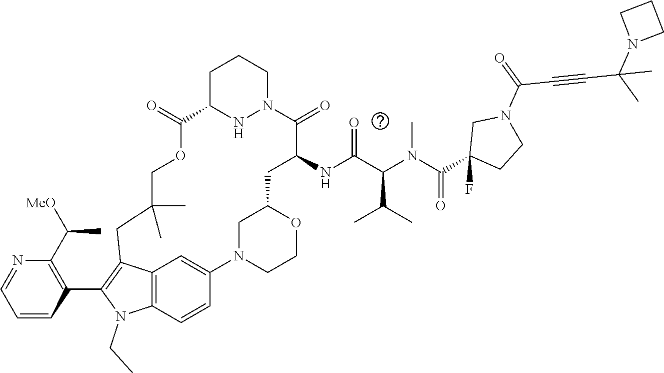 |
| A727 | 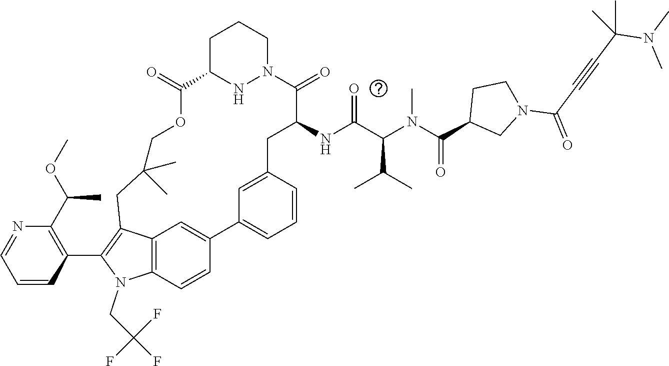 |
| A728 | 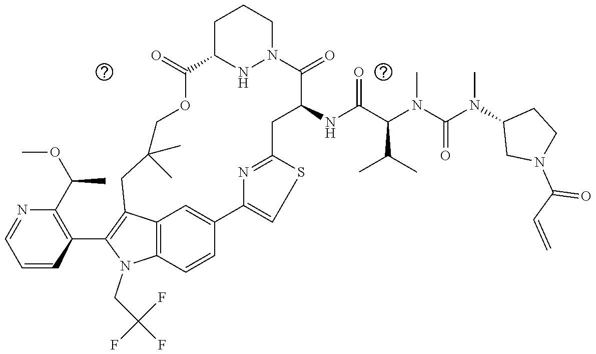 |
| A729 | 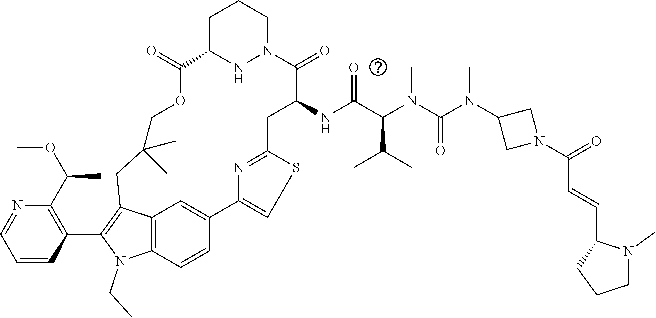 |
| A730 | 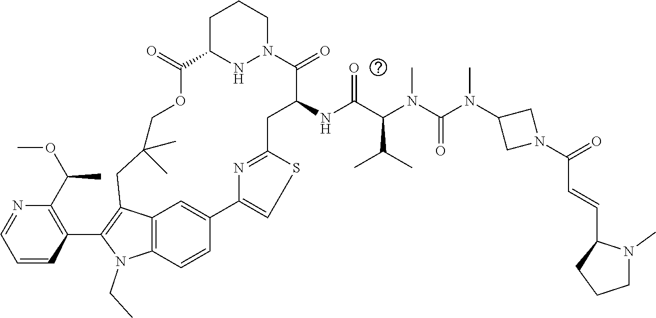 |
| A731 | 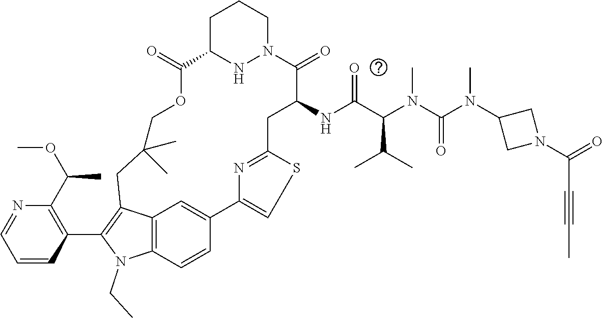 |
| A732 | 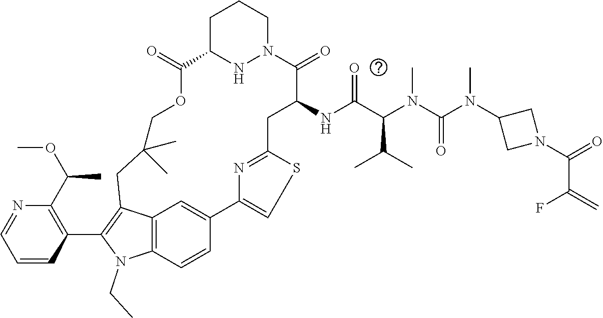 |
| A733 | 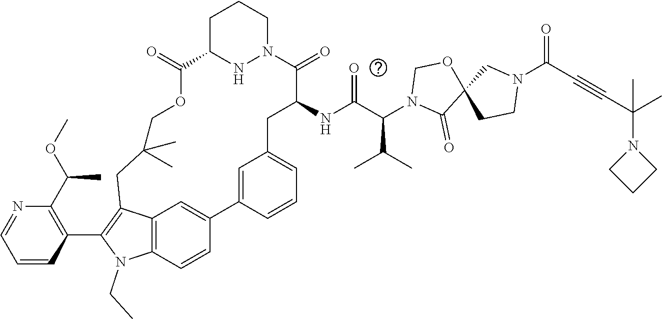 |
| A734 | 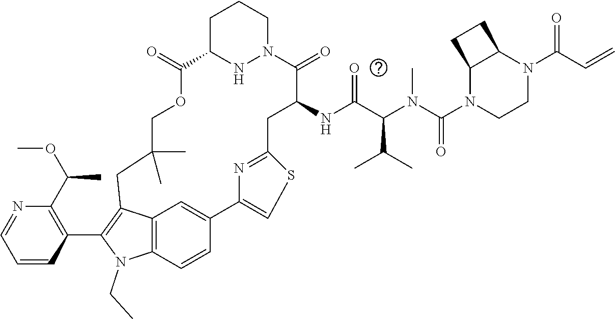 |
| A735 | 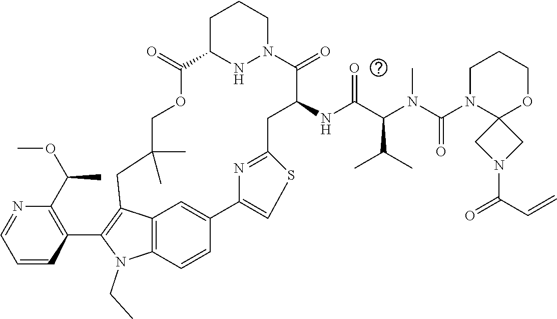 |
| A736 | 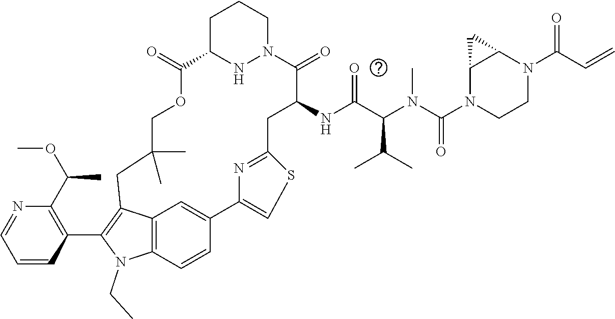 |
| A737 | 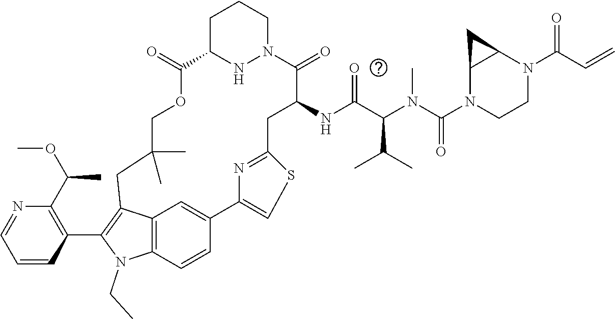 |
| A738 | 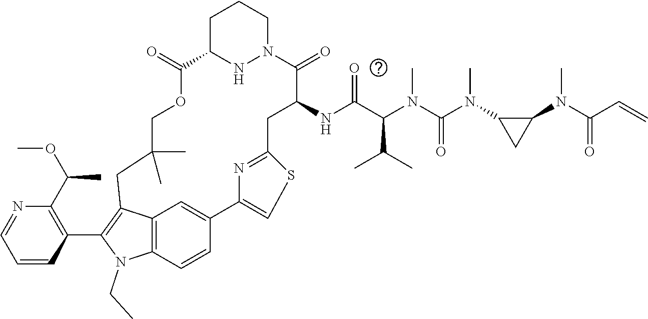 |
| A739 | 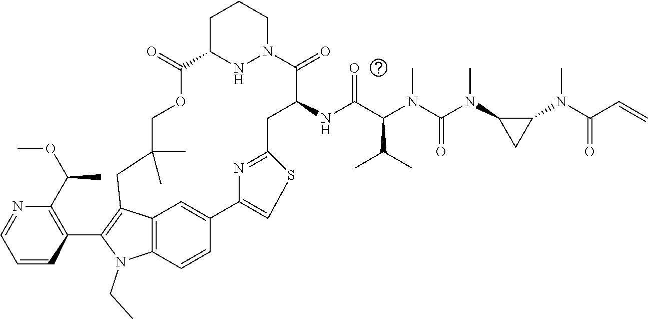 |
| A740 | 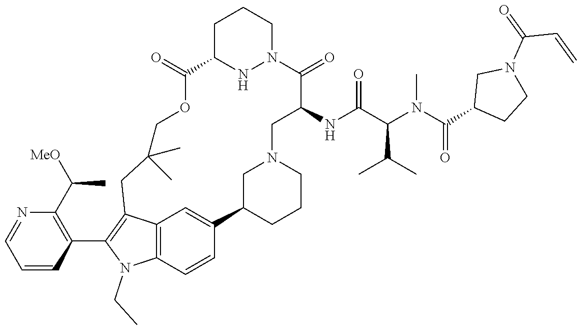 |
| A741 | 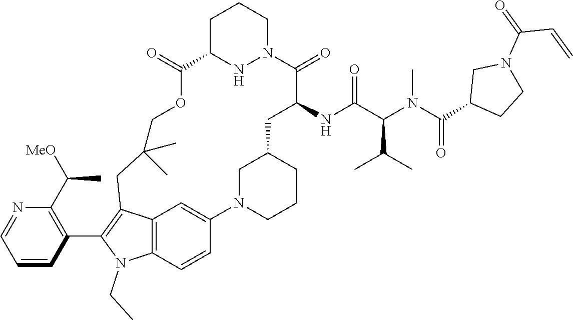 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.
Brackts are to be ignored.
*The activity of this stereoisomer may, in fact, be attributable to the presence of a small amount of the stereoisomer with the (S) configuration at the
—NC(O)—CH(CH₃)₂—N(CH₃)— position.

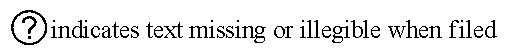

In some embodiments, a compound of Table 2 is provided, or a pharmaceutically acceptable salt thereof. In some embodiments, a compound of the present invention is selected from Table 2, or a pharmaceutically acceptable salt or atropisomer thereof.

TABLE 2
Certain Compounds of the Present Invention

| Ex# | Structure |
|---|---|
| B1 | 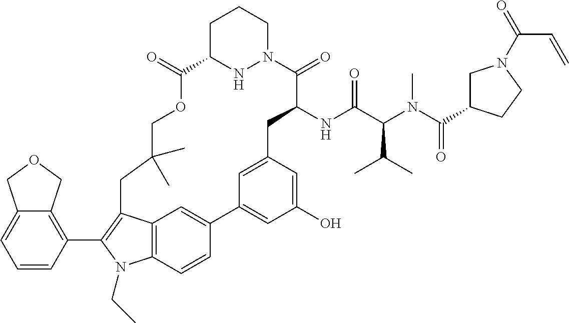 [SMILES: C=CC(=O)N1CC[C@H](C(=O)N(C)[C@H](C(=O)N[C@H]2Cc3cc(O)cc(c3)-c3ccc4c(c3)c(c(-c3cccc5c3COC5)n4CC)CC(C)(C)COC(=O)[C@@H]3CCCN(N3)C2=O)C(C)C)C1] |
| B2 | 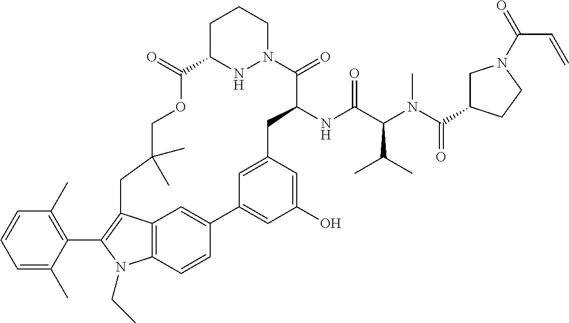 [SMILES: C=CC(=O)N1CC[C@H](C(=O)N(C)[C@H](C(=O)N[C@H]2Cc3cc(O)cc(c3)-c3ccc4c(c3)c(c(-c3c(C)cccc3C)n4CC)CC(C)(C)COC(=O)[C@@H]3CCCN(N3)C2=O)C(C)C)C1] |
| B3 | 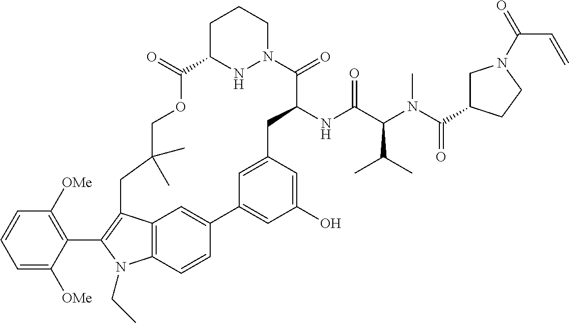 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cccc3OC)n4CC)CC(C)(C)COC(=O)[C@@H]3CCCN(N3)C2=O)C(C)C)C1] |
| B4 | 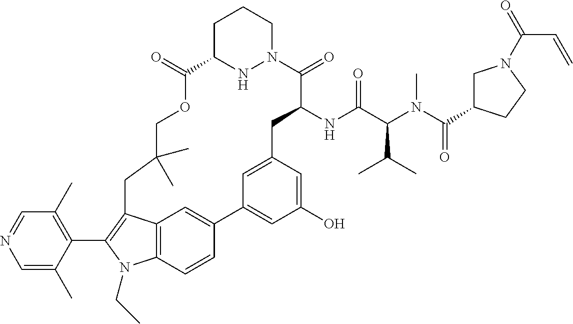 [SMILES: C=CC(=O)N1CC[C@H](C(=O)N(C)[C@H](C(=O)N[C@H]2Cc3cc(O)cc(c3)-c3ccc4c(c3)c(c(-c3c(C)cncc3C)n4CC)CC(C)(C)COC(=O)[C@@H]3CCCN(N3)C2=O)C(C)C)C1] |
| B5 | 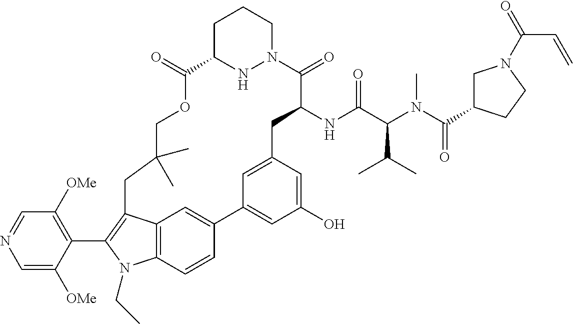 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cncc3OC)n4CC)CC(C)(C)COC(=O)[C@@H]3CCCN(N3)C2=O)C(C)C)C1] |
| B6 | 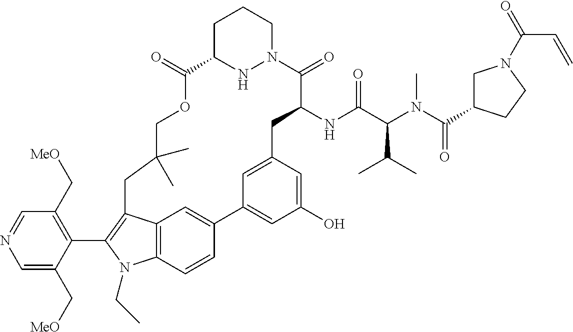 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cncc3COC)n4CC)CC(C)(C)COC(=O)[C@@H]3CCCN(N3)C2=O)C(C)C)C1] |
| B7 | 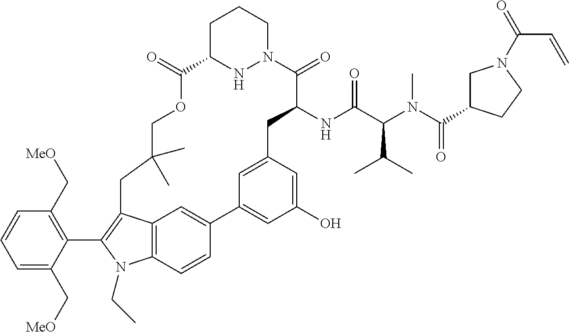 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cccc3COC)n4CC)CC(C)(C)COC(=O)[C@@H]3CCCN(N3)C2=O)C(C)C)C1] |
| B11 | 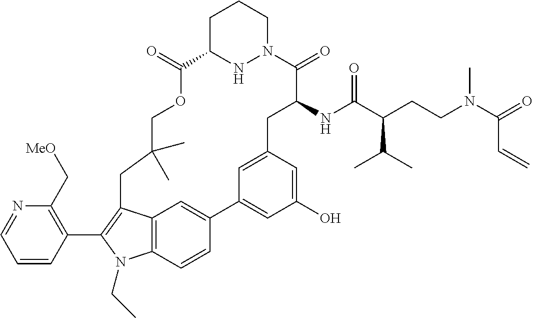 [SMILES: C=CC(=O)N(C)CC[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| B12 | 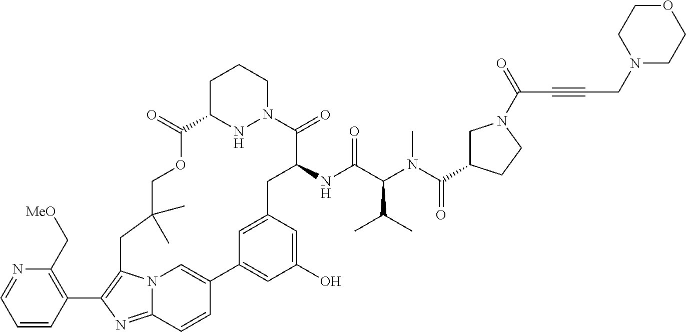 [SMILES: COCc1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| B13 | 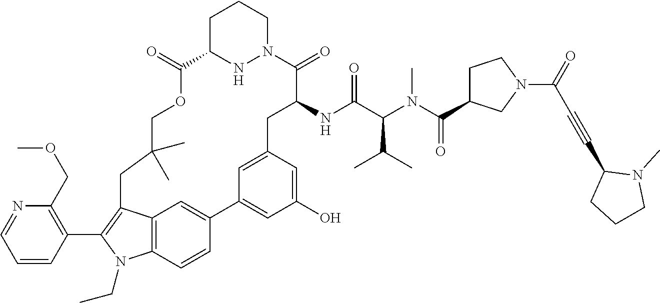 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| B18 | 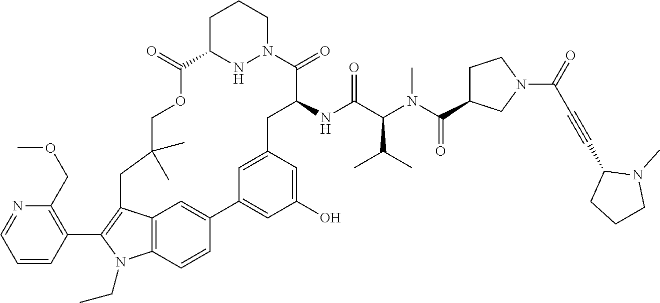 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H]3CCCN3C)C1)C(=O)N1CCC[C@H](N1)C(=O)OCC(C)(C)C2] |
| B21 | 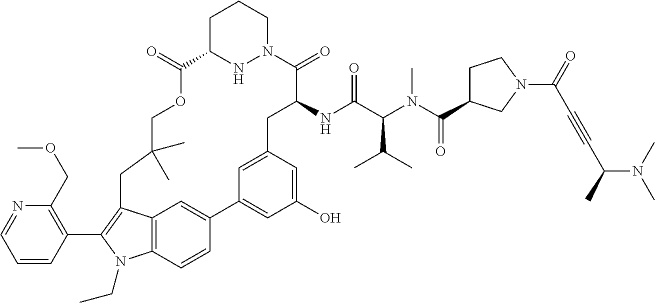 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H](C)N(C)C)C1)C(=O)N1CCC[C@H](N1)C(=O)OCC(C)(C)C2] |
| B22 | 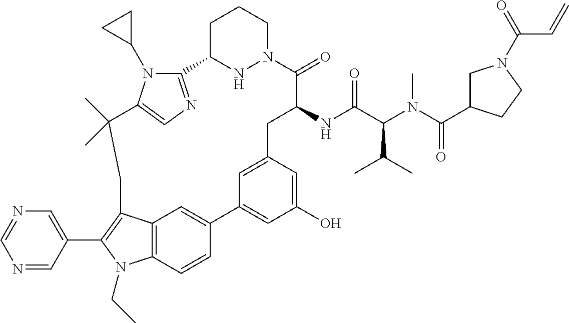 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)c3cnc(n3C3CC3)[C@@H]3CCCN(N3)C2=O)C(C)C)C1] |
| B25 | 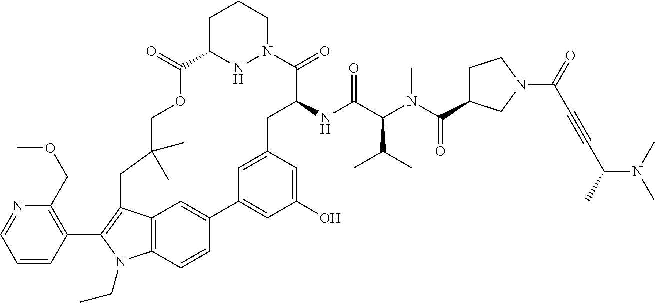 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H](C)N(C)C)C1)C(=O)N1CCC[C@H](N1)C(=O)OCC(C)(C)C2] |
| B27 | 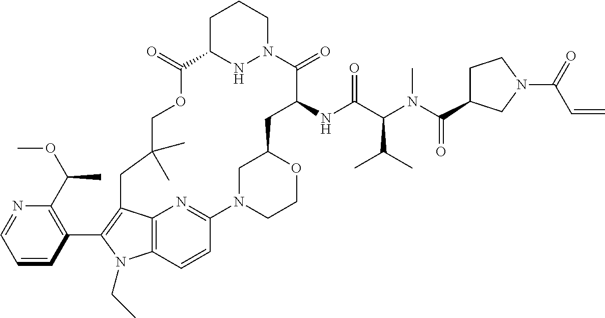 |
| B28 | 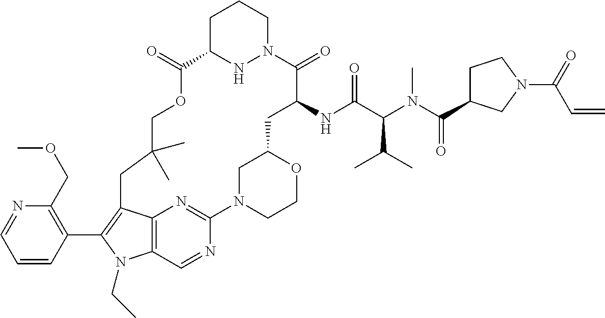 [SMILES: C=CC(=O)N1CC[C@H](C(=O)N(C)[C@H](C(=O)N[C@H]2C[C@H]3CN(CCO3)c3ncc4c(n3)c(c(-c3cccnc3COC)n4CC)CC(C)(C)COC(=O)[C@@H]3CCCN(N3)C2=O)C(C)C)C1] |
| B29 | 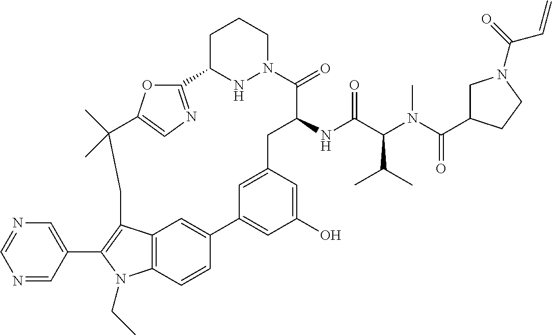 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3=CN=C(O3)[C@@H]3CCCN(N3)C2=O)C(C)C)C1] |
| B30 | 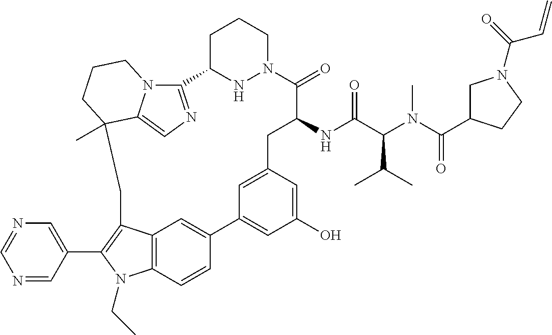 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC3(C)CCCn4c3cnc4[C@@H]3CCCN(N3)C2=O)C(C)C)C1] |
| B32 | 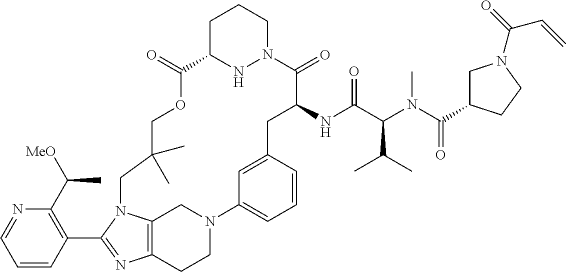 [SMILES: C=CC(=O)N1CC[C@H](C(=O)N(C)[C@H](C(=O)N[C@H]2Cc3cccc(c3)N3CCc4nc(-c5cccnc5[C@H](C)OC)n(c4C3)CC(C)(C)COC(=O)[C@@H]3CCCN(N3)C2=O)C(C)C)C1] |
| B34 | 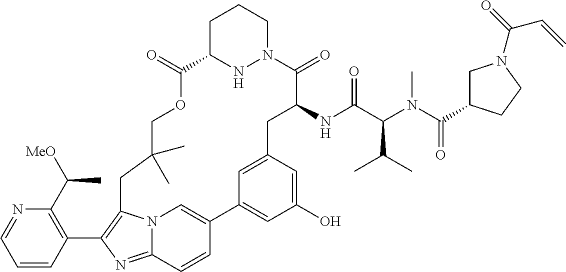 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)c(n4c3)CC(C)(C)COC(=O)[C@@H]3CCCN(N3)C2=O)C(C)C)C1] |
| B38 | 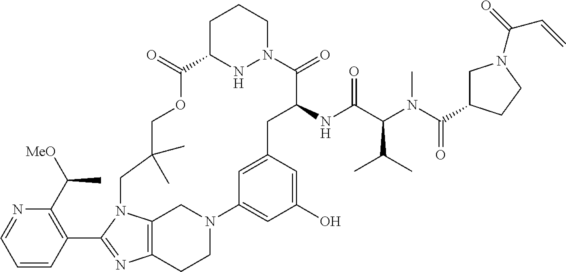 [SMILES: C=CC(=O)N1CC[C@H](C(=O)N(C)[C@H](C(=O)N[C@H]2Cc3cc(O)cc(c3)N3CCc4nc(-c5cccnc5[C@H](C)OC)n(c4C3)CC(C)(C)COC(=O)[C@@H]3CCCN(N3)C2=O)C(C)C)C1] |
| B47 | 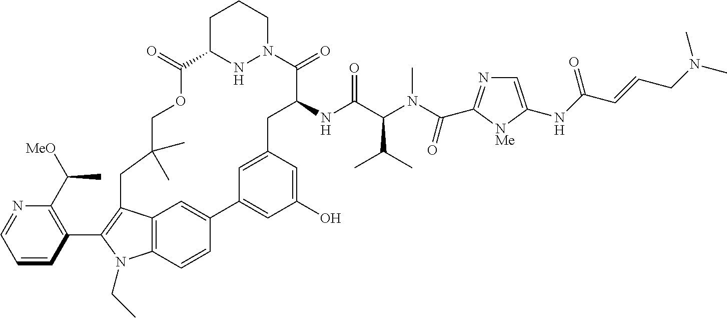 |
| B64 | 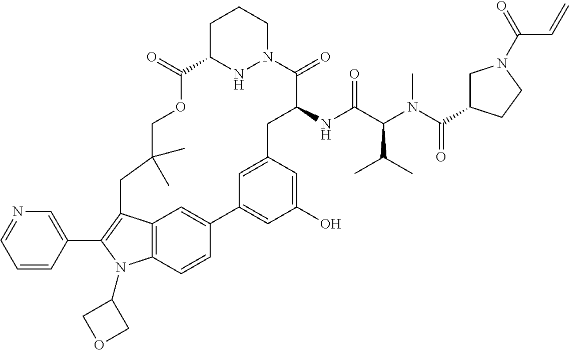 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3COC3)CC(C)(C)COC(=O)[C@@H]3CCCN(N3)C2=O)C(C)C)C1] |
| B65 | 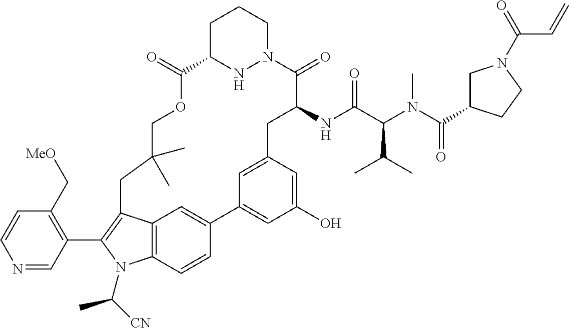 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4[C@H](C)C#N)CC(C)(C)COC(=O)[C@@H]3CCCN(N3)C2=O)C(C)C)C1] |
| B66 | 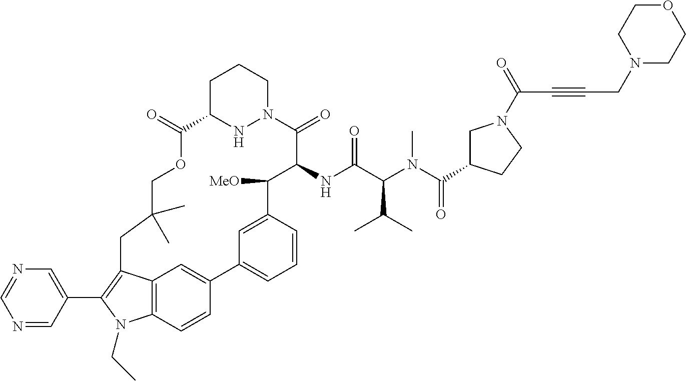 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B70 | 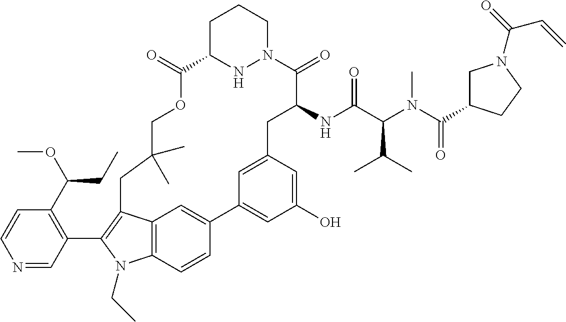 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@H](CC)OC)n4CC)CC(C)(C)COC(=O)[C@@H]3CCCN(N3)C2=O)C(C)C)C1] |
| B73 | 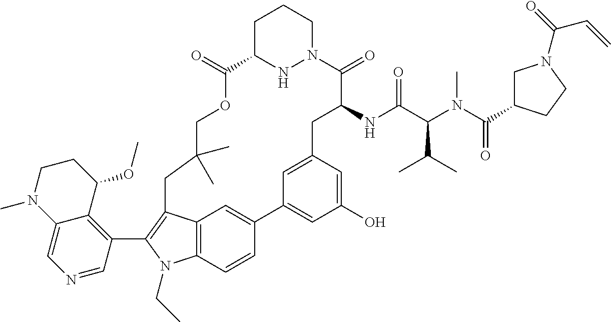 [SMILES: C=CC(=O)N1CC[C@H](C(=O)N(C)[C@H](C(=O)N[C@H]2Cc3cc(O)cc(c3)-c3ccc4c(c3)c(c(-c3cncc5c3[C@@H](OC)CCN5C)n4CC)CC(C)(C)COC(=O)[C@@H]3CCCN(N3)C2=O)C(C)C)C1] |
| B74 | 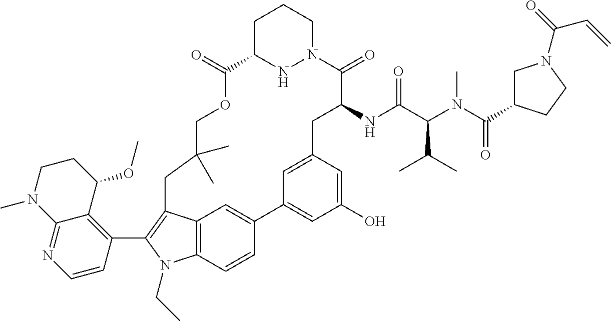 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CCN5C)n4CC)CC(C)(C)COC(=O)[C@@H]3CCCN(N3)C2=O)C(C)C)C1] |
| B75 | 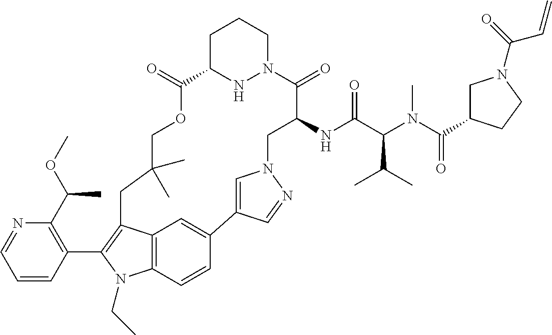 [SMILES: C=CC(=O)N1CC[C@H](C(=O)N(C)[C@H](C(=O)N[C@H]2Cn3cc(cn3)-c3ccc4c(c3)c(c(-c3cccnc3[C@H](C)OC)n4CC)CC(C)(C)COC(=O)[C@@H]3CCCN(N3)C2=O)C(C)C)C1] |
| B76 | 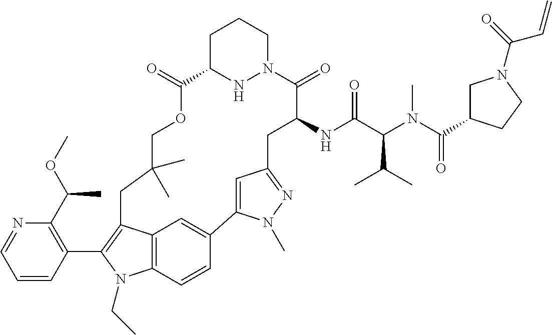 [SMILES: C=CC(=O)N1CC[C@H](C(=O)N(C)[C@H](C(=O)N[C@H]2Cc3cc(n(C)n3)-c3ccc4c(c3)c(c(-c3cccnc3[C@H](C)OC)n4CC)CC(C)(C)COC(=O)[C@@H]3CCCN(N3)C2=O)C(C)C)C1] |
| B77 | 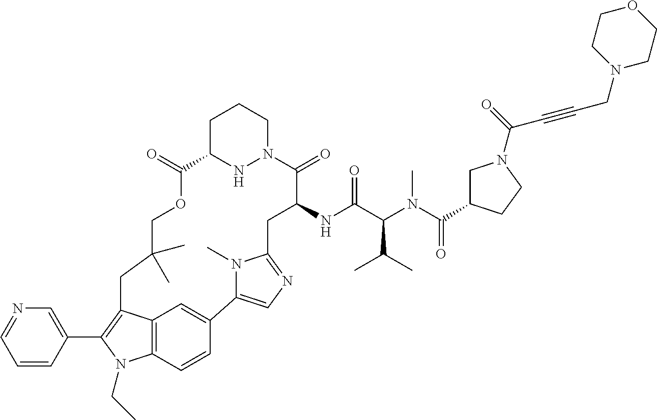 [SMILES: CCn1c(-c2cccnc2)c2c3cc(ccc31)-c1cnc(n1C)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B81 | 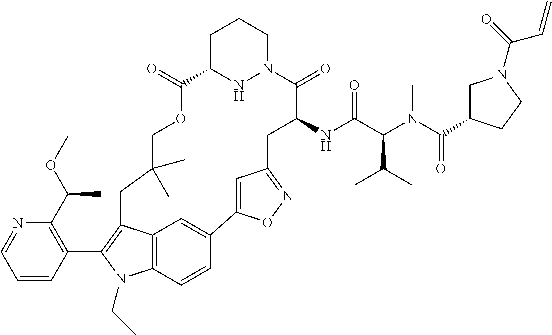 [SMILES: C=CC(=O)N1CC[C@H](C(=O)N(C)[C@H](C(=O)N[C@H]2Cc3cc(on3)-c3ccc4c(c3)c(c(-c3cccnc3[C@H](C)OC)n4CC)CC(C)(C)COC(=O)[C@@H]3CCCN(N3)C2=O)C(C)C)C1] |
| B83 | 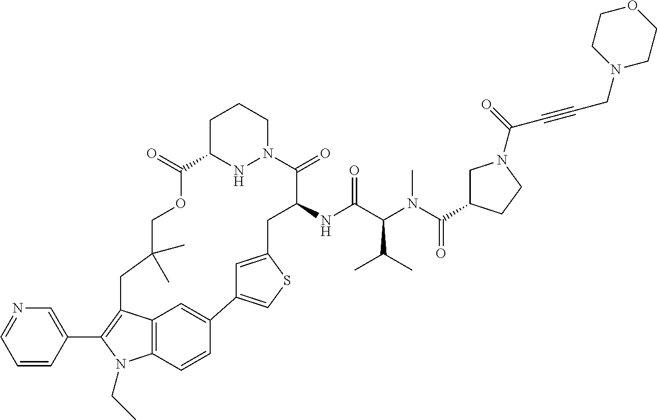 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B85 | 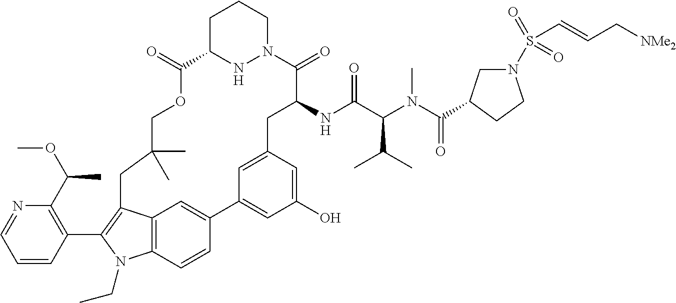 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)/C=C/CN(C)C)C1)C(=O)N1CCC[C@H](N1)C(=O)OCC(C)(C)C2] |
| B86 | 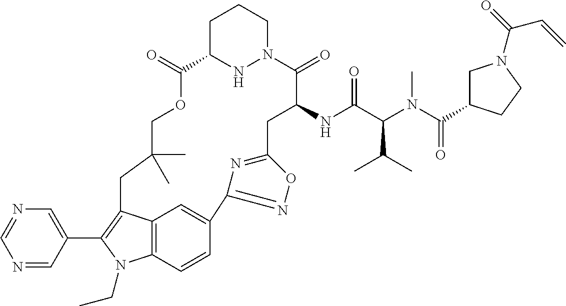 [SMILES: C=CC(=O)N1CC[C@H](C(=O)N(C)[C@H](C(=O)N[C@H]2Cc3nc(no3)-c3ccc4c(c3)c(c(-c3cncnc3)n4CC)CC(C)(C)COC(=O)[C@@H]3CCCN(N3)C2=O)C(C)C)C1] |
| B87 | 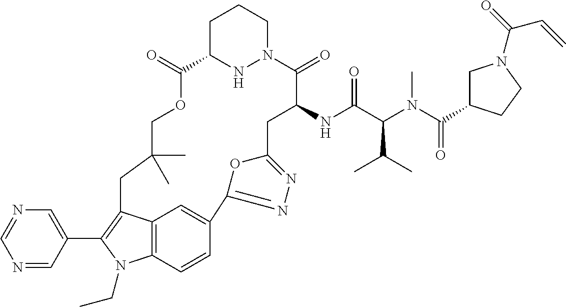 [SMILES: C=CC(=O)N1CC[C@H](C(=O)N(C)[C@H](C(=O)N[C@H]2CC3=NN=C(O3)c3ccc4c(c3)c(c(-c3cncnc3)n4CC)CC(C)(C)COC(=O)[C@@H]3CCCN(N3)C2=O)C(C)C)C1] |
| B88 | 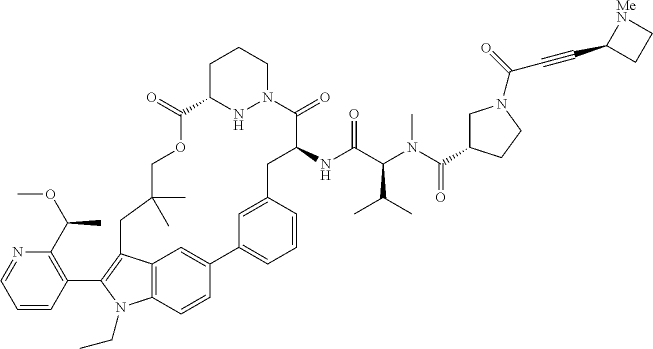 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| B89 | 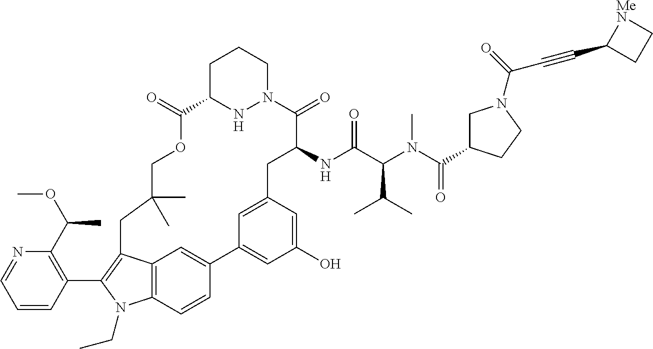 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| B90 | 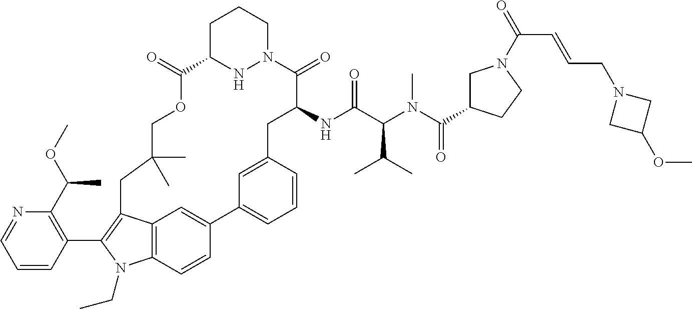 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)/C=C/CN3CC(OC)C3)C1)C(=O)N1CCC[C@H](N1)C(=O)OCC(C)(C)C2] |
| B91 | 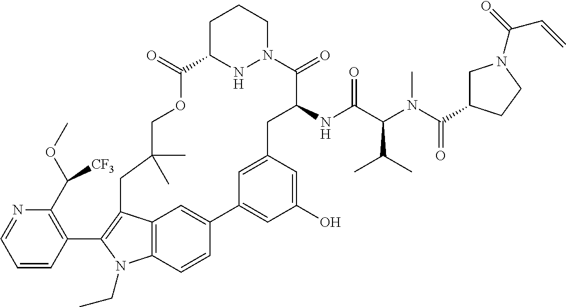 [SMILES: C=CC(=O)N1CC[C@H](C(=O)N(C)[C@H](C(=O)N[C@H]2Cc3cc(O)cc(c3)-c3ccc4c(c3)c(c(-c3cccnc3[C@@H](OC)C(F)(F)F)n4CC)CC(C)(C)COC(=O)[C@@H]3CCCN(N3)C2=O)C(C)C)C1] |
| B96 | 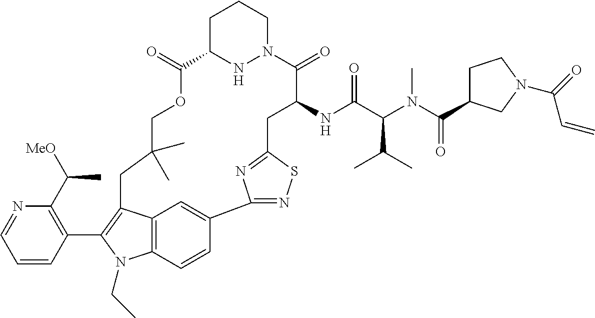 [SMILES: C=CC(=O)N1CC[C@H](C(=O)N(C)[C@H](C(=O)N[C@H]2Cc3nc(ns3)-c3ccc4c(c3)c(c(-c3cccnc3[C@H](C)OC)n4CC)CC(C)(C)COC(=O)[C@@H]3CCCN(N3)C2=O)C(C)C)C1] |
| B97 | 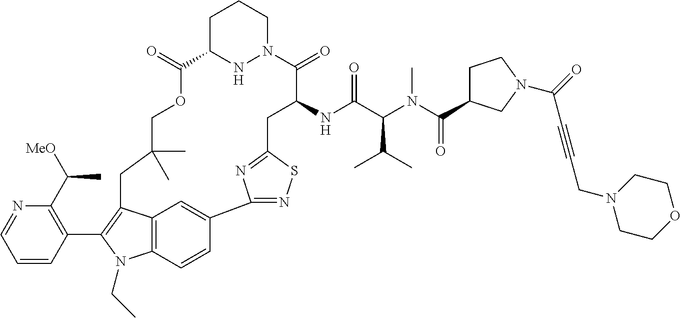 [SMILES: CCn1c(-c2cccnc2[C@H](C)OC)c2c3cc(ccc31)-c1nsc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B102 | 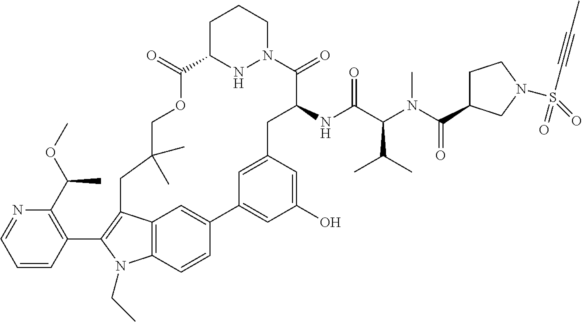 [SMILES: CC#CS(=O)(=O)N1CC[C@H](C(=O)N(C)[C@H](C(=O)N[C@H]2Cc3cc(O)cc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| B103 | 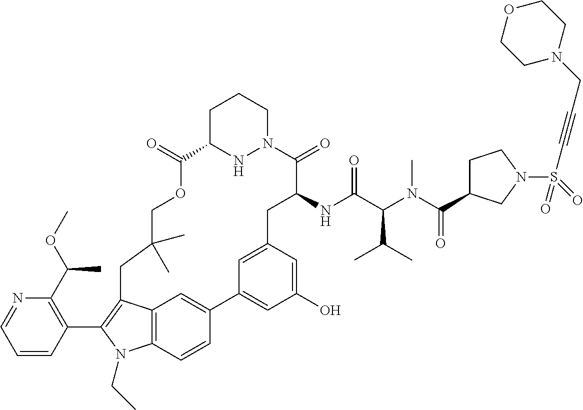 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)C#CCN3CCOCC3)C1)C(=O)N1CCC[C@H](N1)C(=O)OCC(C)(C)C2] |
| B104 | 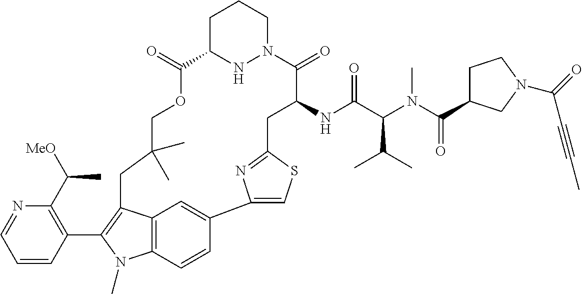 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C)CC(C)(C)COC(=O)[C@@H]3CCCN(N3)C2=O)C(C)C)C1] |
| B106 | 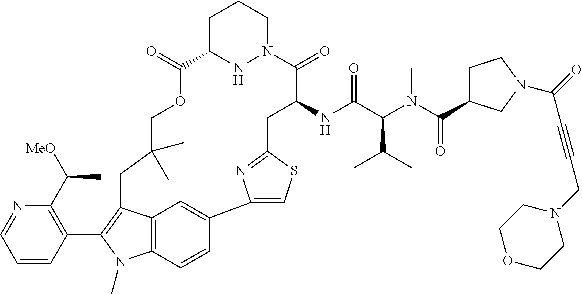 [SMILES: CO[C@@H](C)c1ncccc1-c1c2c3cc(ccc3n1C)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B107 | 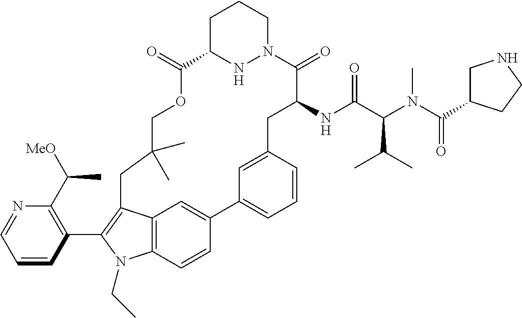 |
| B109 | 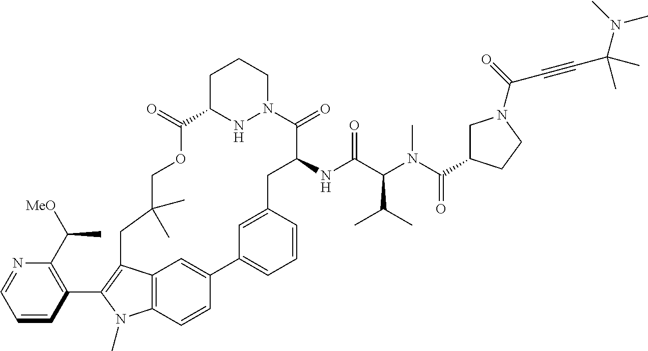 |
| B111 | 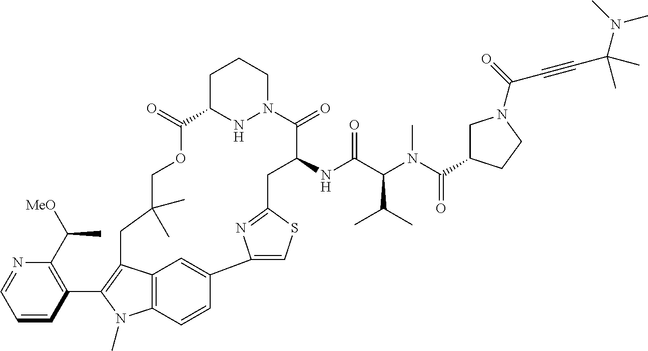 |
| B112 | 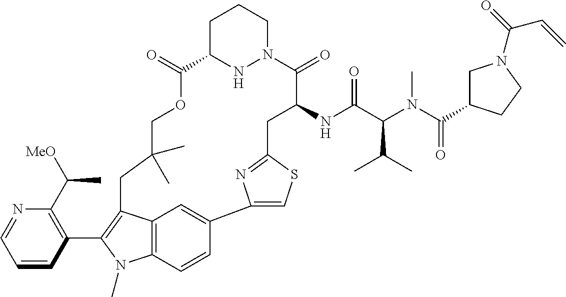 |
| B113 | 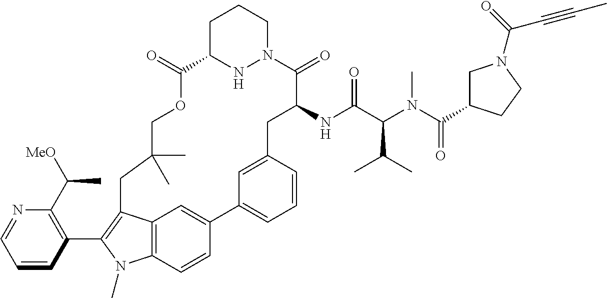 |
| B115 | 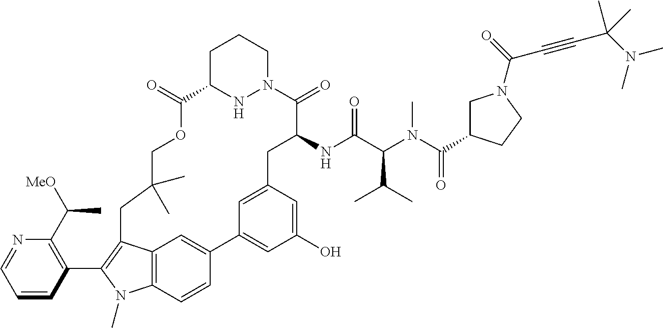 |
| B116 | 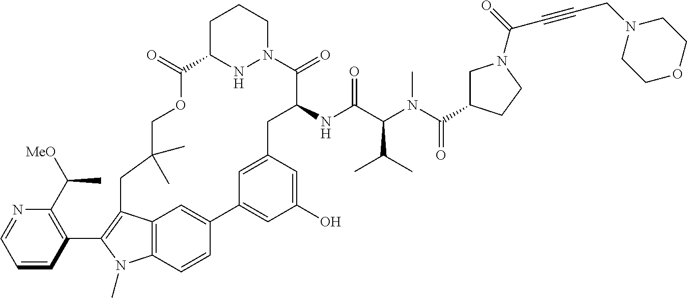 |
| B117 | 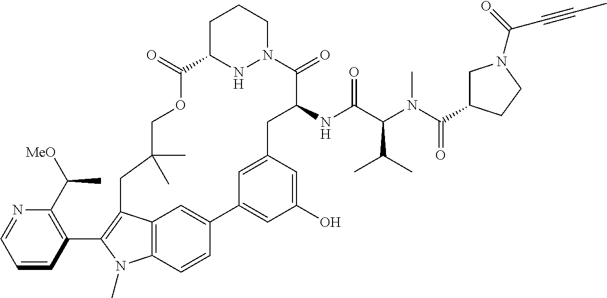 |
| B118 | 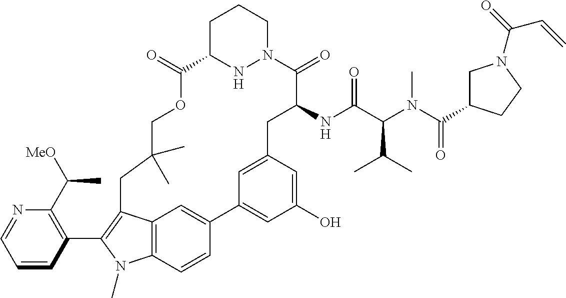 |
| B119 | 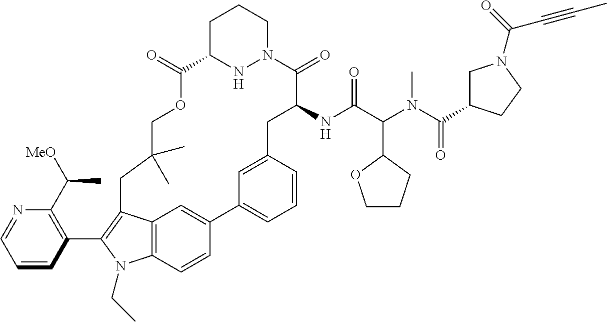 |
| B120 | 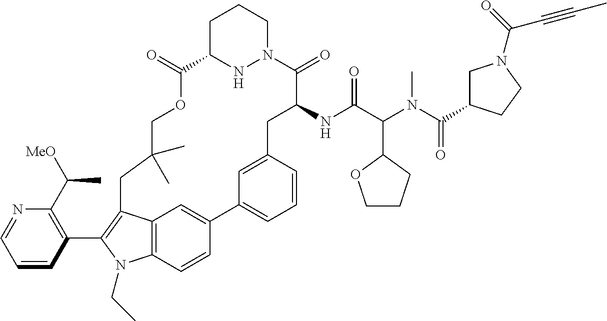 |
| B121 | 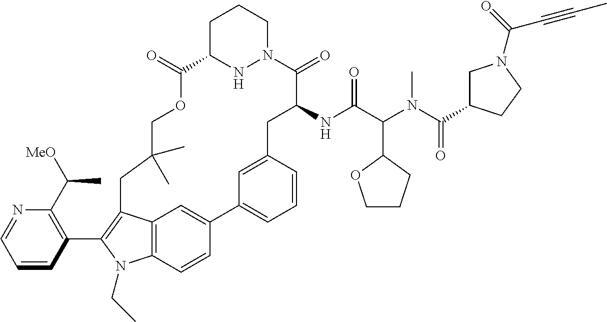 |
| B122 | 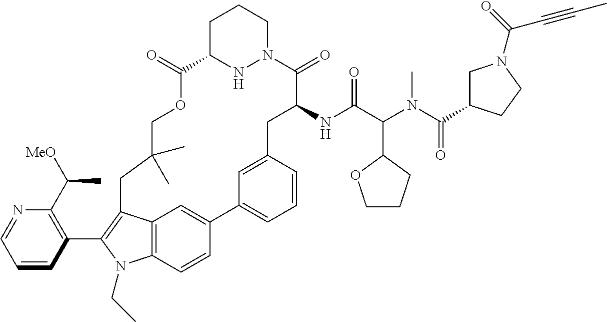 |
| B123 | 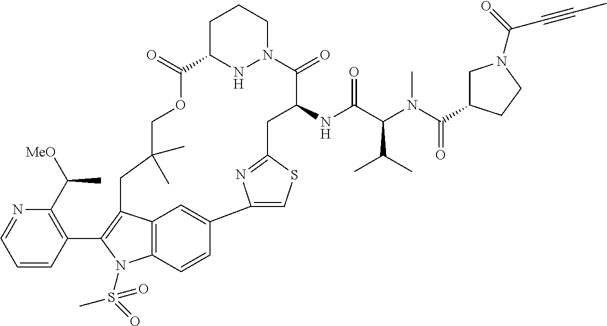 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4S(C)(=O)=O)CC(C)(C)COC(=O)[C@@H]3CCCN(N3)C2=O)C(C)C)C1] |
| B124 | 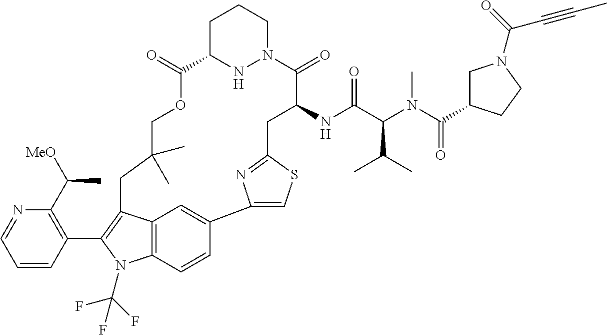 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(F)(F)F)CC(C)(C)COC(=O)[C@@H]3CCCN(N3)C2=O)C(C)C)C1] |
| B126 | 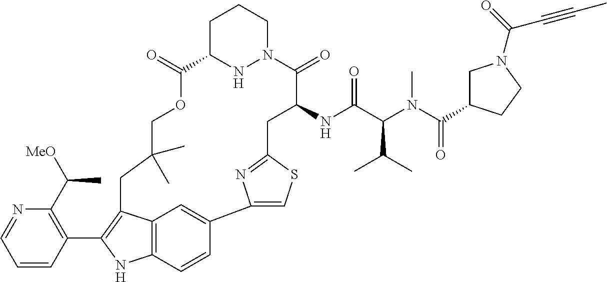 [SMILES: CC#CC(=O)N1CC[C@H](C(=O)N(C)[C@H](C(=O)N[C@H]2Cc3nc(cs3)-c3ccc4[nH]c(-c5cccnc5[C@H](C)OC)c(c4c3)CC(C)(C)COC(=O)[C@@H]3CCCN(N3)C2=O)C(C)C)C1] |
| B127 | 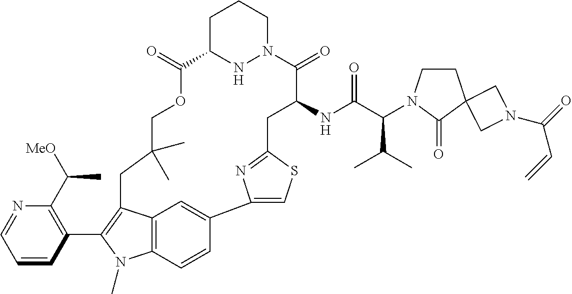 |
| B128 | 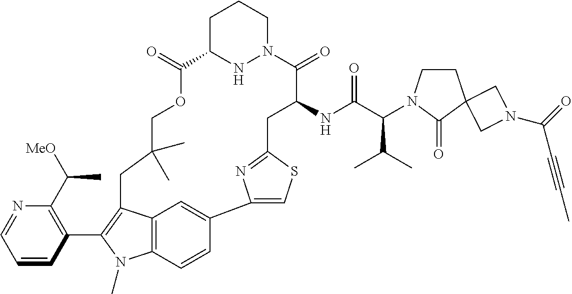 |
| B129 | 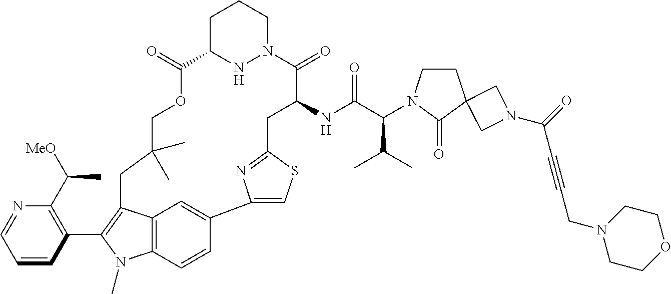 |
| B130 | 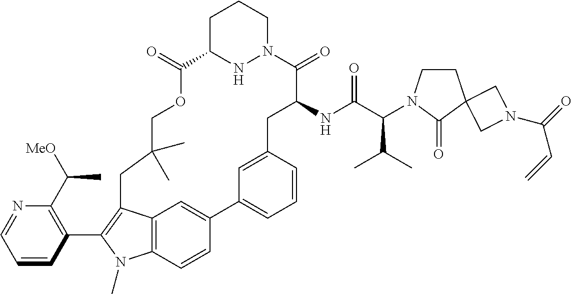 |
| B131 | 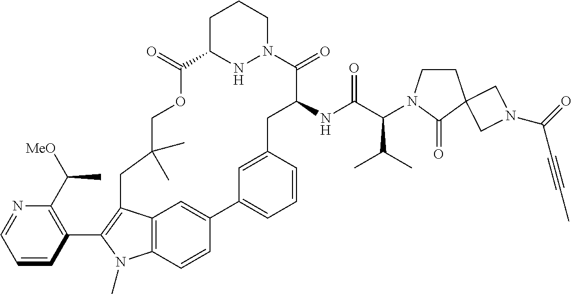 |
| B132 | 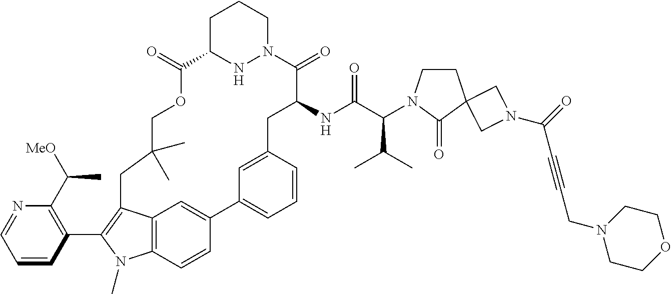 |
| B139 | 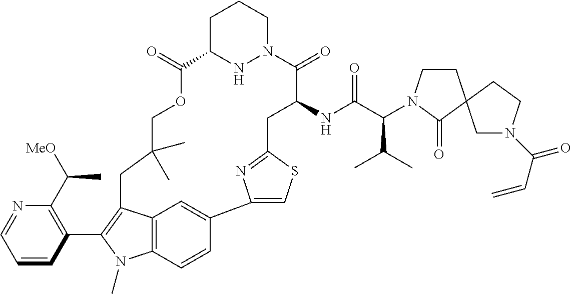 |
| B140 | 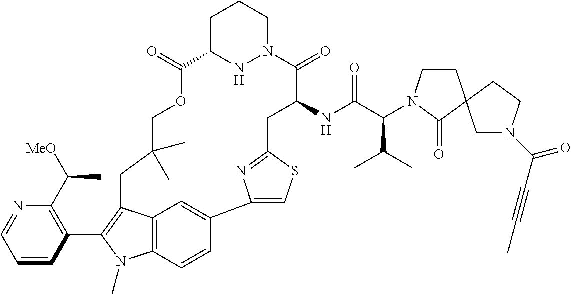 |
| B141 | 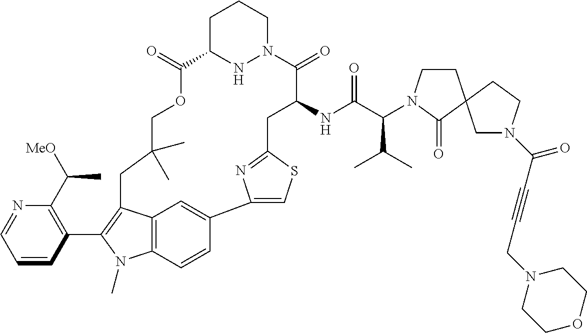 |
| B142 | 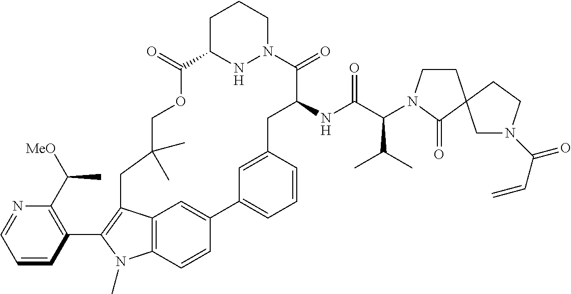 |
| B143 | 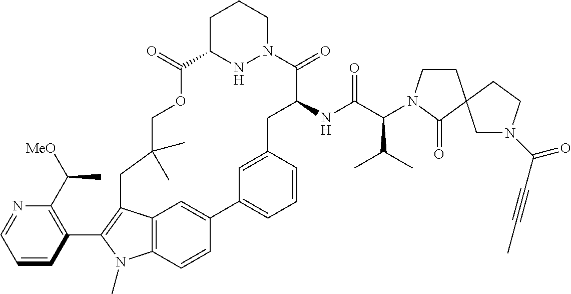 |
| B144 | 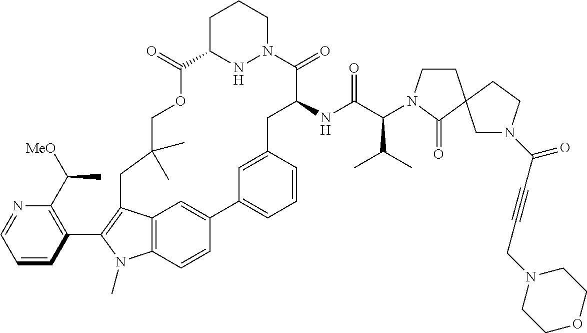 |
| B145 | 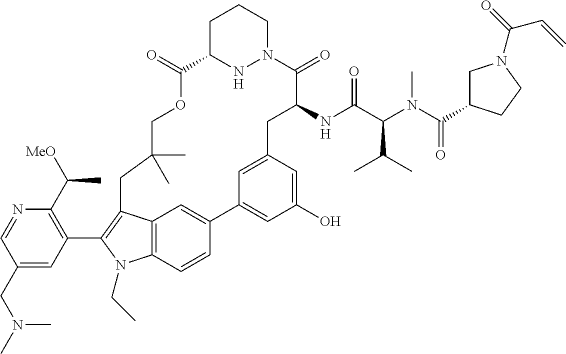 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| B146 | 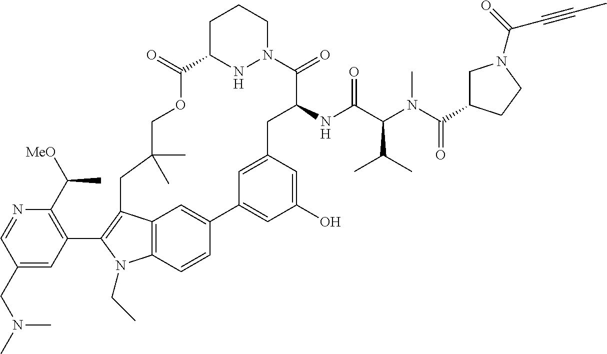 [SMILES: CC#CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| B147 | 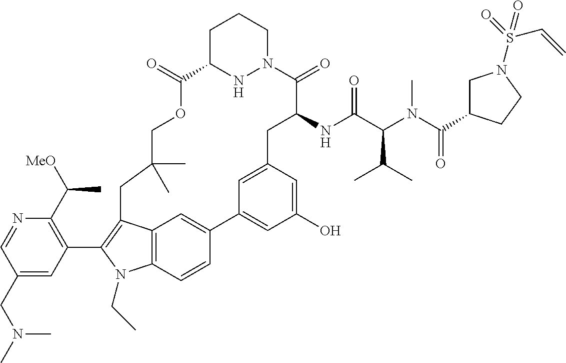 [SMILES: C=CS(=O)(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| B148 | 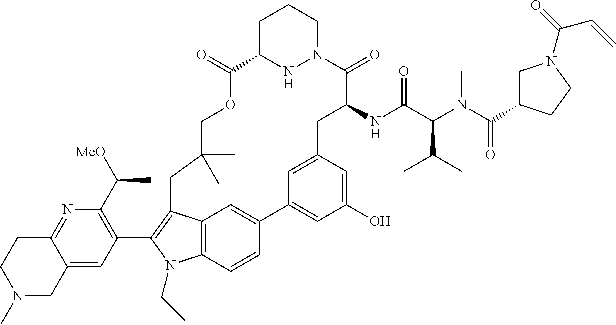 [SMILES: C=CC(=O)N1CC[C@H](C(=O)N(C)[C@H](C(=O)N[C@H]2Cc3cc(O)cc(c3)-c3ccc4c(c3)c(c(-c3cc5c(nc3[C@H](C)OC)CCN(C)C5)n4CC)CC(C)(C)COC(=O)[C@@H]3CCCN(N3)C2=O)C(C)C)C1] |
| B149 | 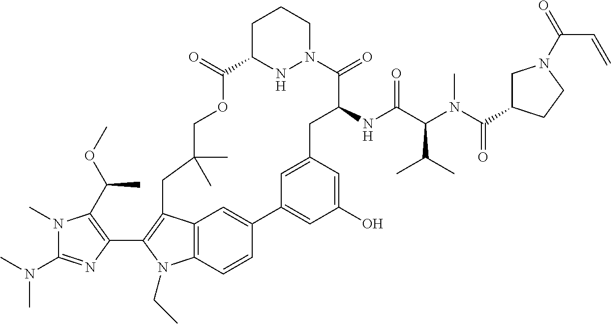 [SMILES: C=CC(=O)N1CC[C@H](C(=O)N(C)[C@H](C(=O)N[C@H]2Cc3cc(O)cc(c3)-c3ccc4c(c3)c(c(-c3nc(N(C)C)n(C)c3[C@H](C)OC)n4CC)CC(C)(C)COC(=O)[C@@H]3CCCN(N3)C2=O)C(C)C)C1] |
| B150 | 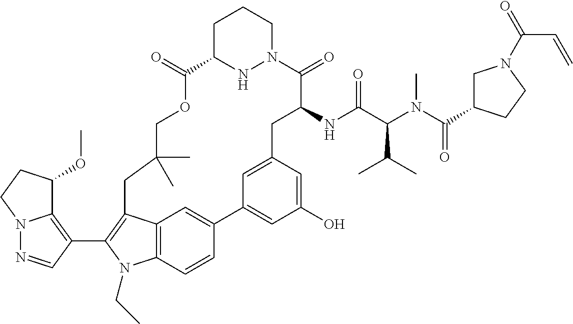 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CC5)n4CC)CC(C)(C)COC(=O)[C@@H]3CCCN(N3)C2=O)C(C)C)C1] |
| B161 | 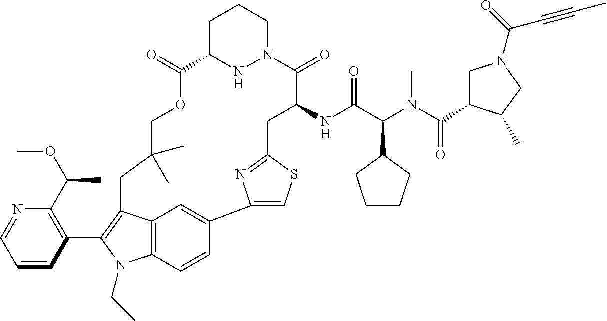 |
| B162 | 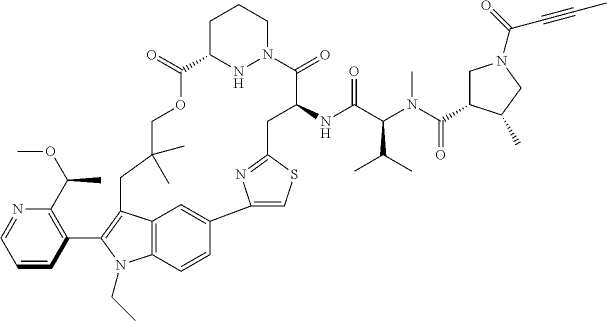 |
| B163 | 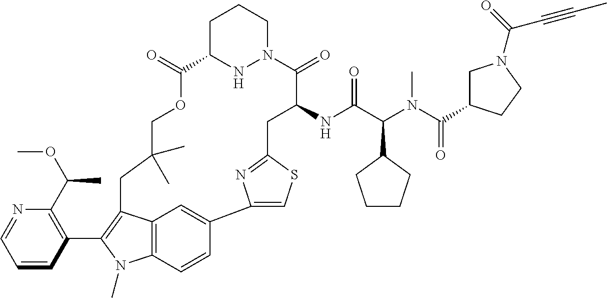 |
| B164 | 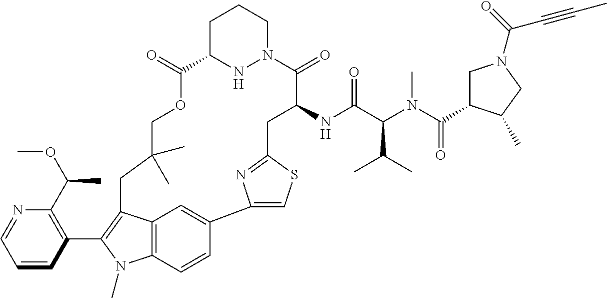 |
| B165 | 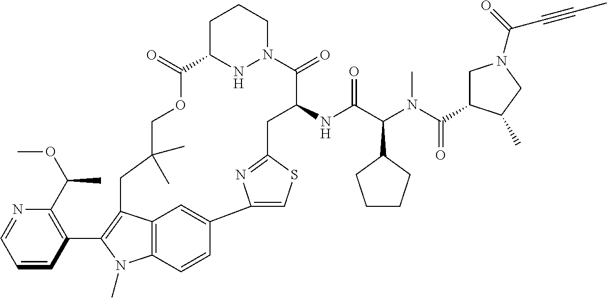 |
| B167 | 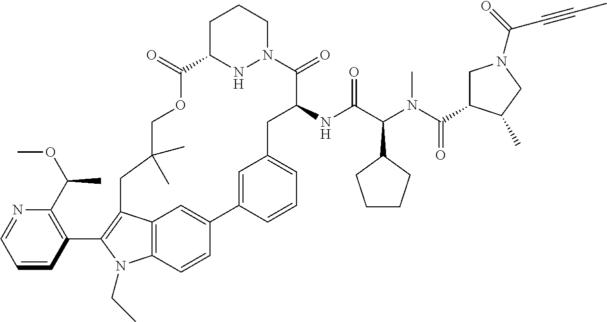 |
| B168 | 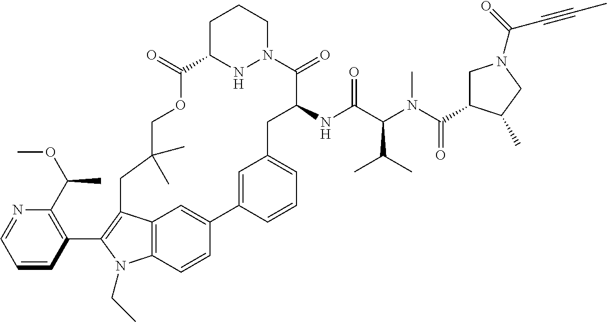 |
| B169 | 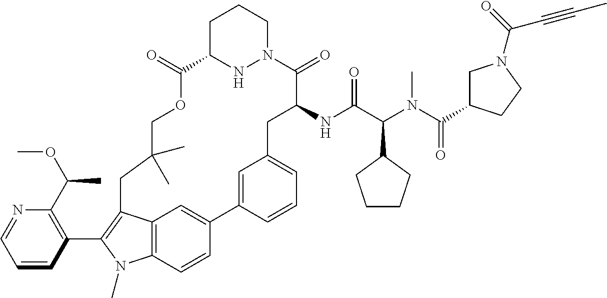 |
| B170 | 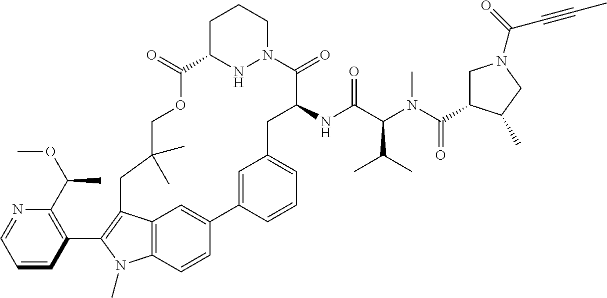 |
| B171 | 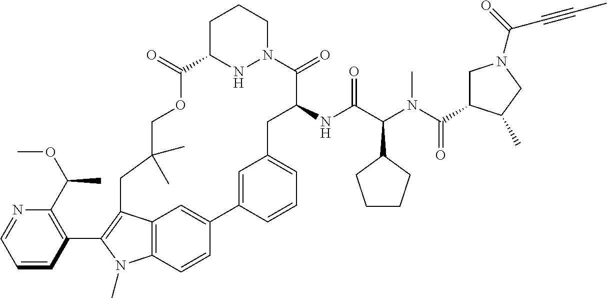 |
| B172 | 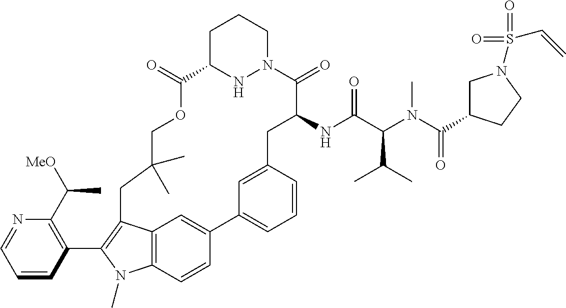 |
| B173 | 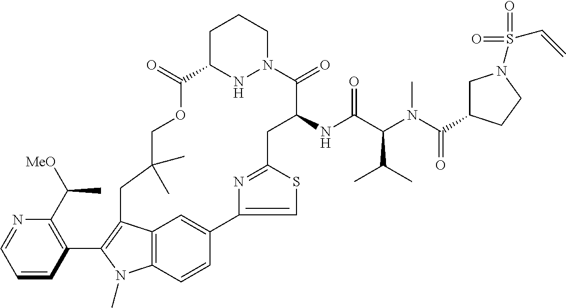 |
| B174 | 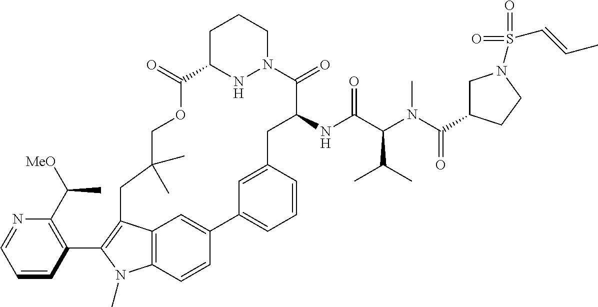 |
| B175 | 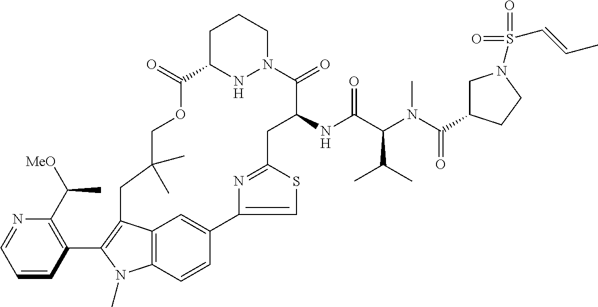 |
| B176 | 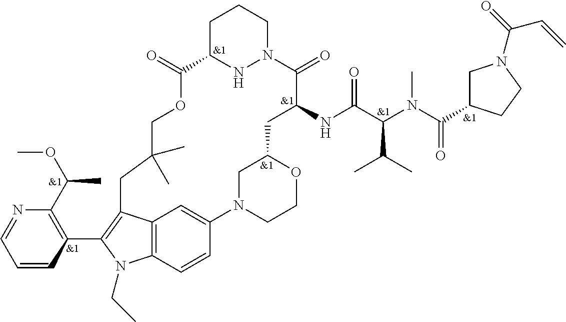 |
| B177 | 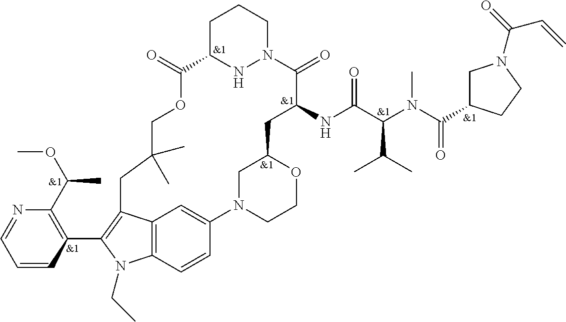 |
| B178 | 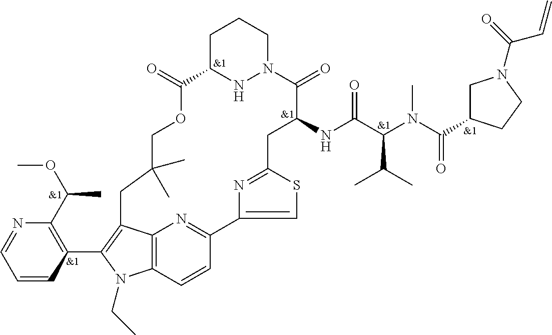 |
| B179 | 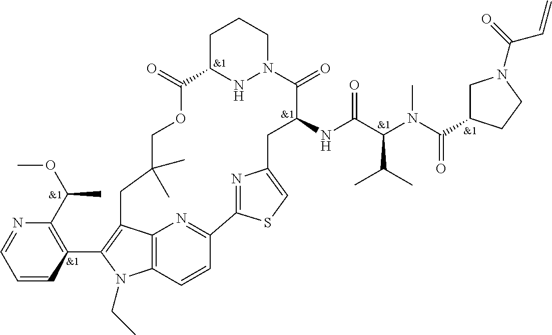 |
| B180 | 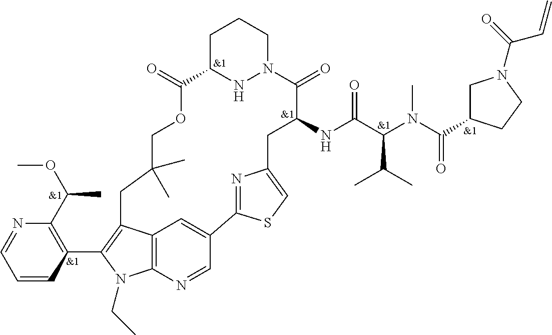 |
| B181 | 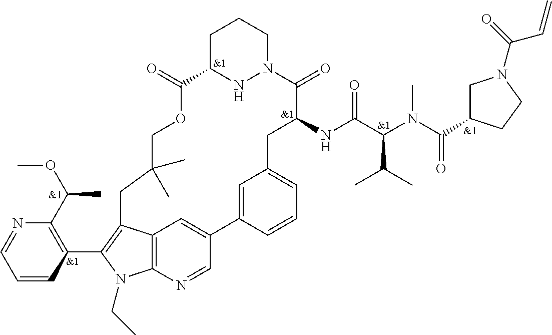 |
| B182 | 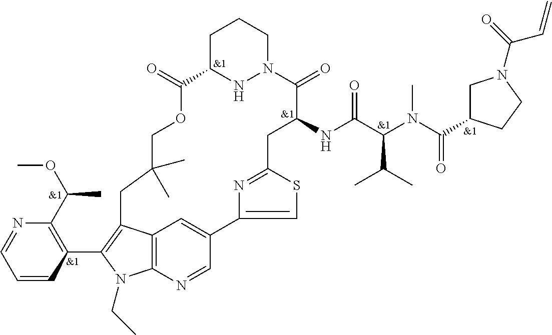 |
| B183 | 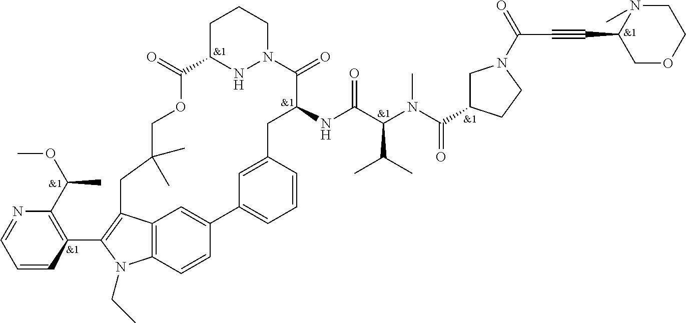 |
| B184 | 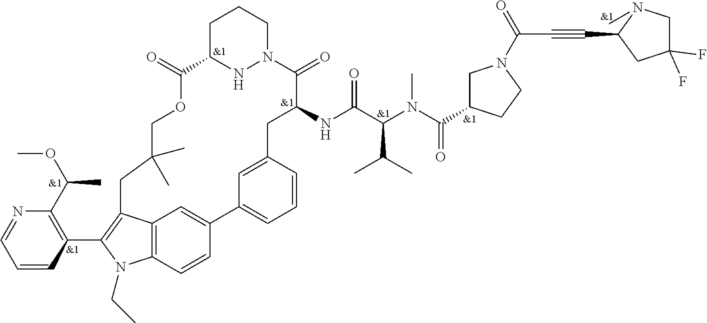 |
| B185 | 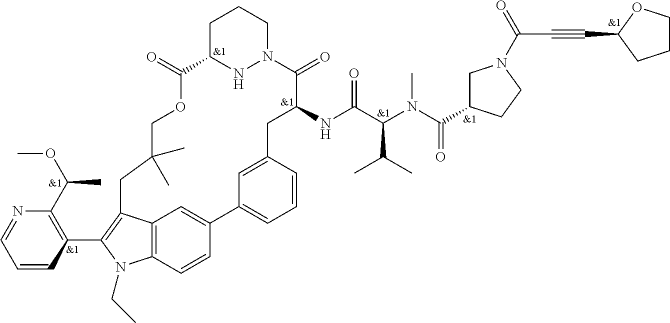 |
| B186 | 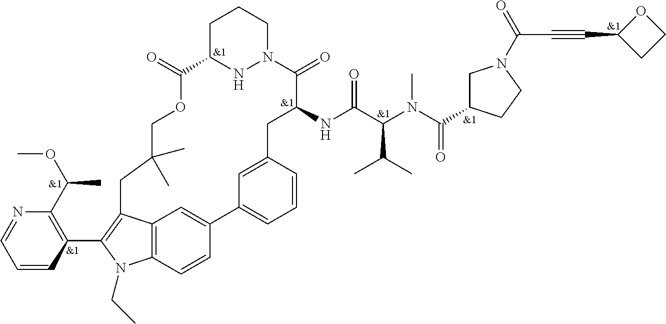 |
| B187 | 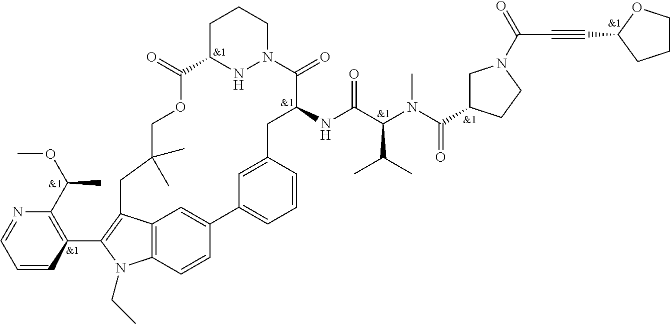 |
| B188 | 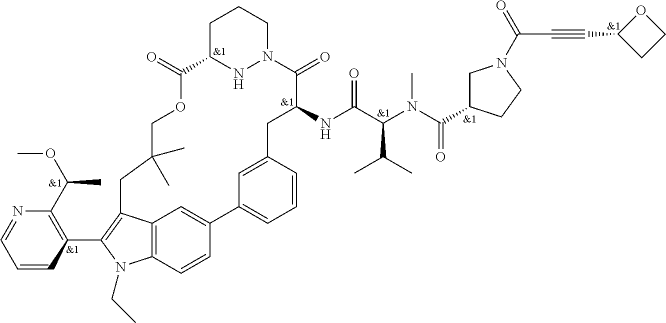 |
| B189 | 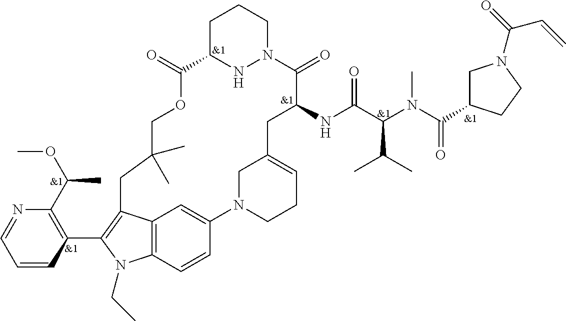 |
| B190 | 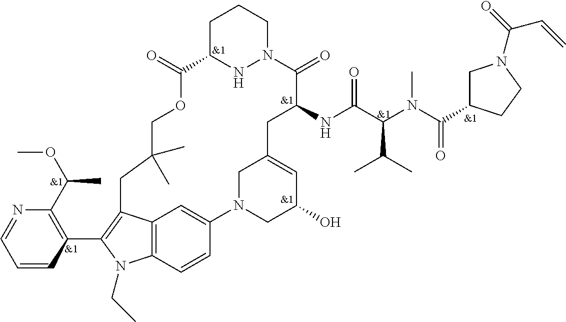 |
| B191 | 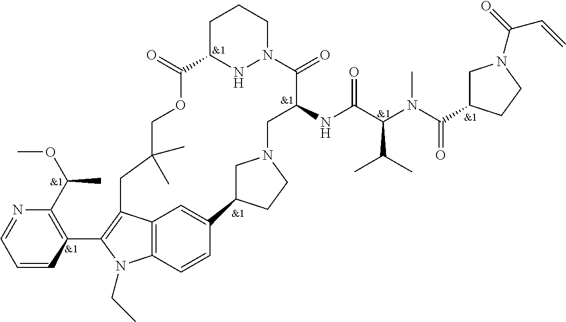 |
| B192 | 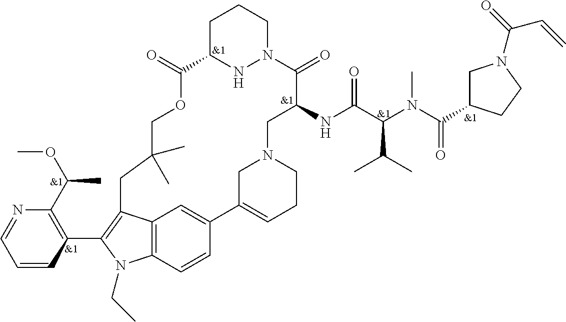 |
| B194 | 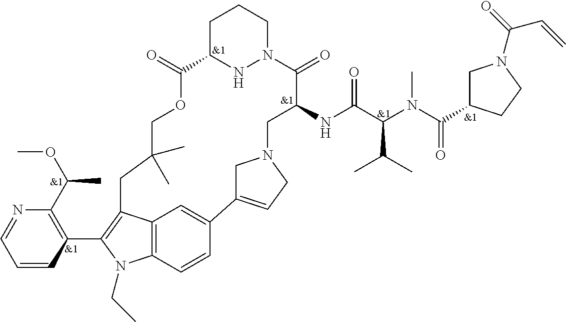 |
| B195 | 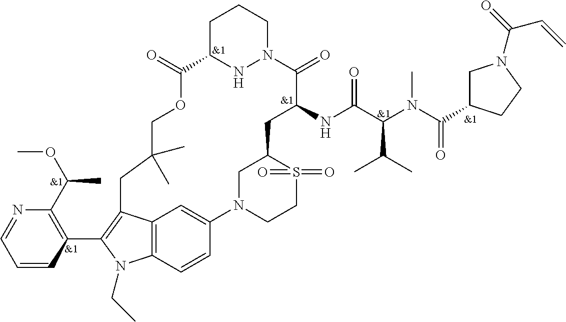 |
| B196 | 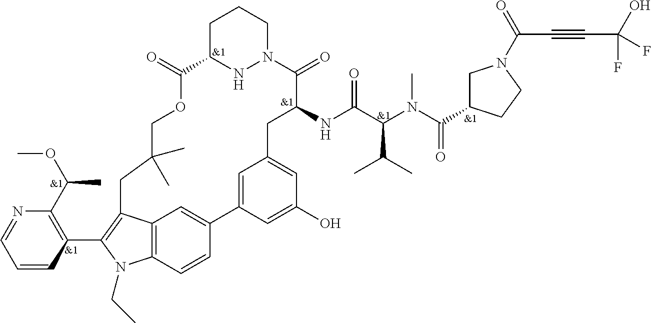 |
| B197 | 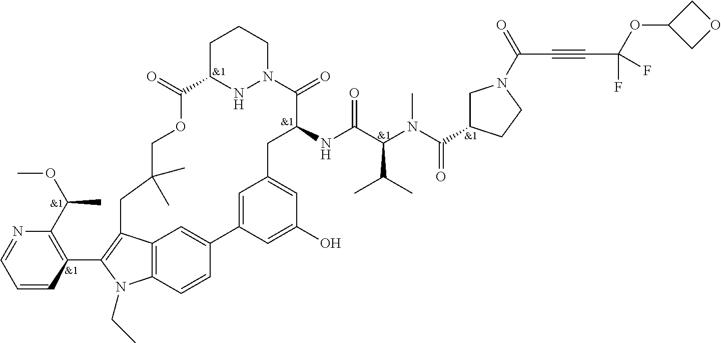 |
| B198 | 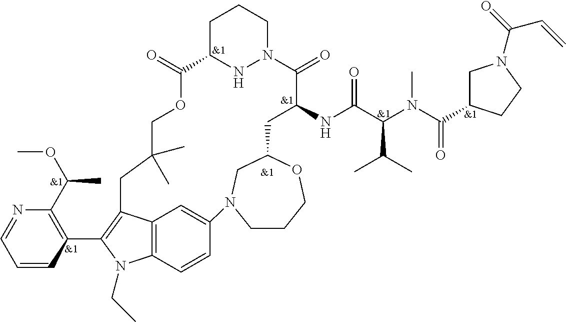 |
| B199 | 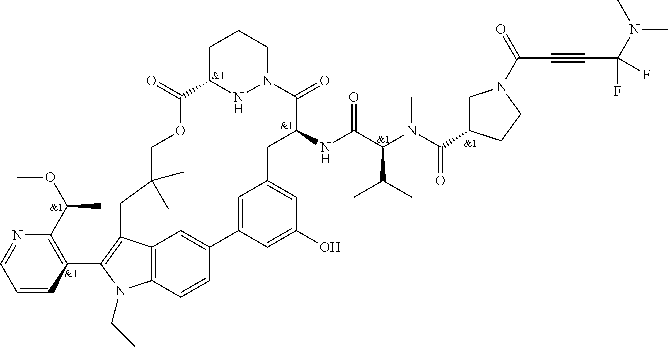 |
| B200 | 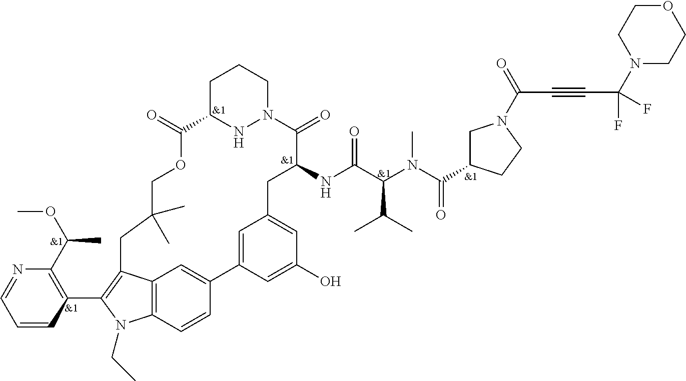 |
| B201 | 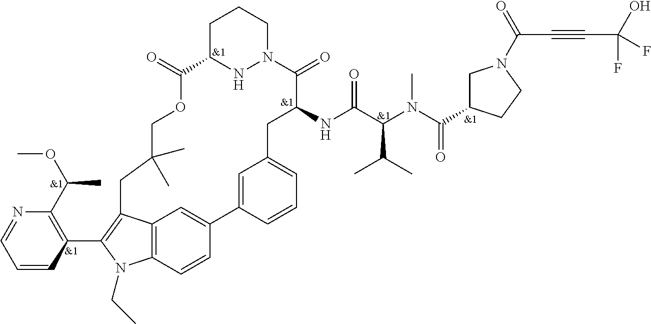 |
| B202 | 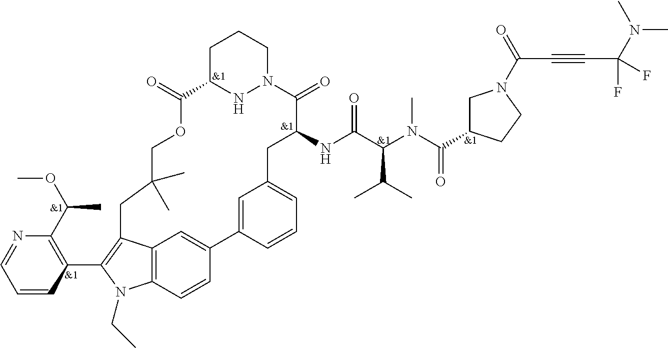 |
| B203 | 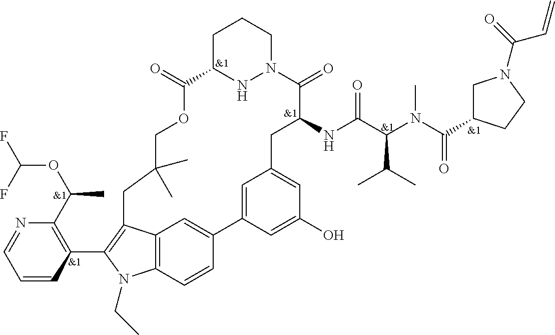 |
| B204 | 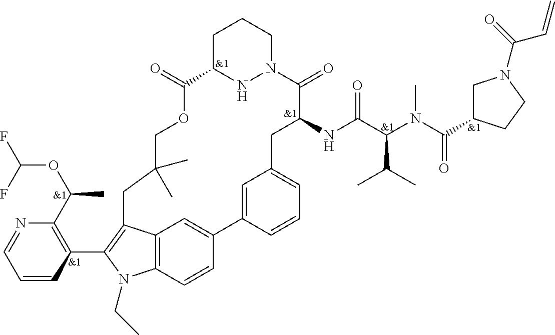 |
| B205 | 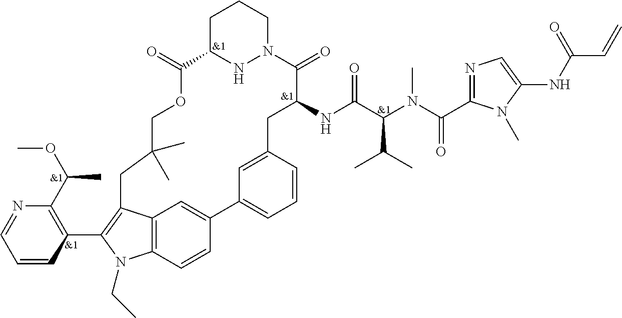 |
| B206 | 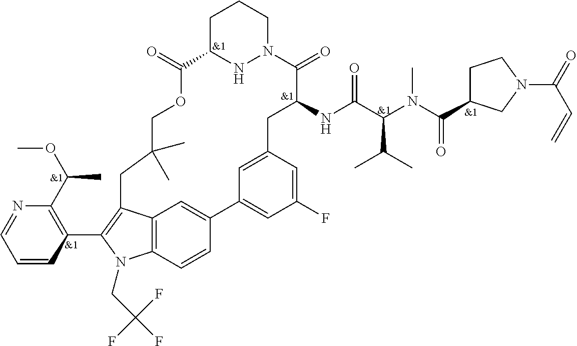 |
| B207 | 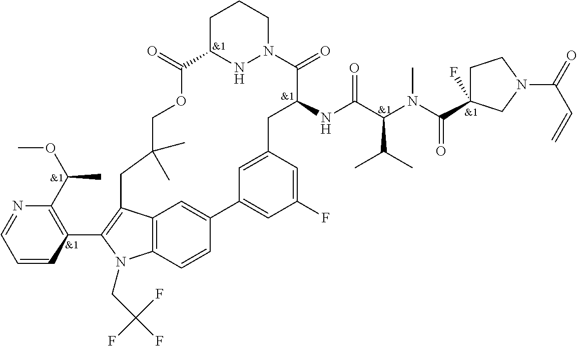 |
| B208 | 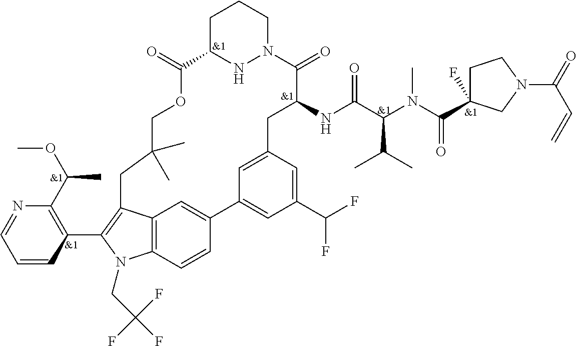 |
| B209 | 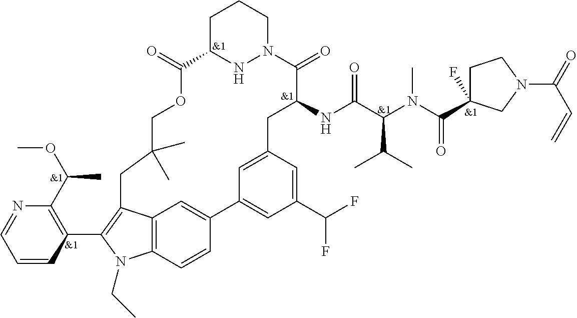 |
| B210 | 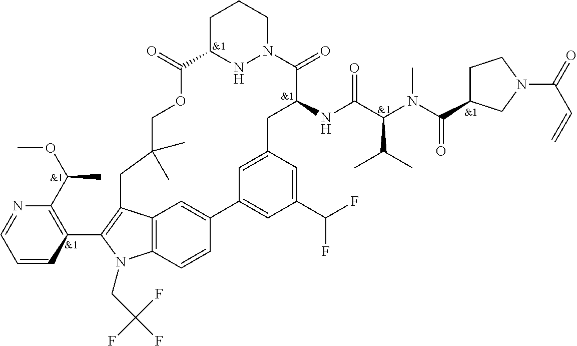 |
| B211 | 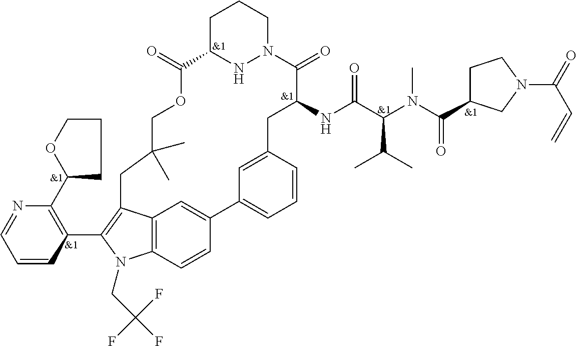 |
| B212 | 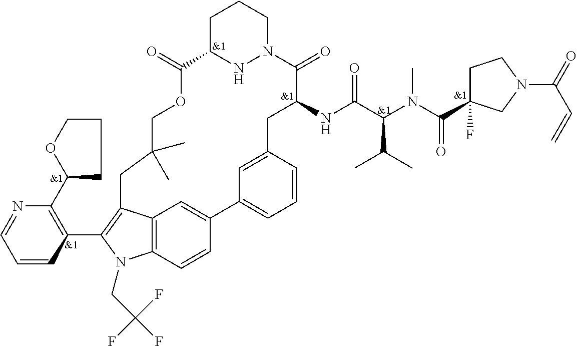 |
| B213 | 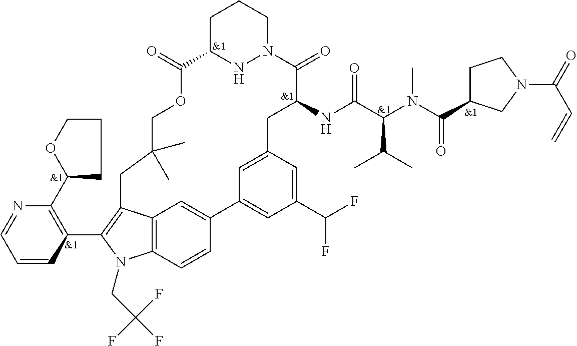 |
| B214 | 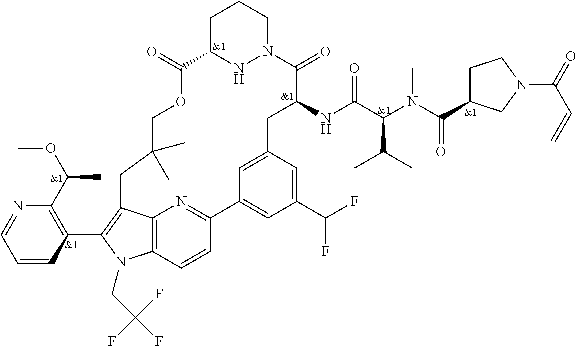 |
| B215 | 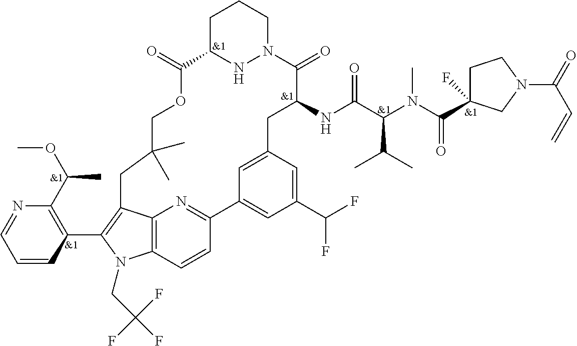 |
| B216 | 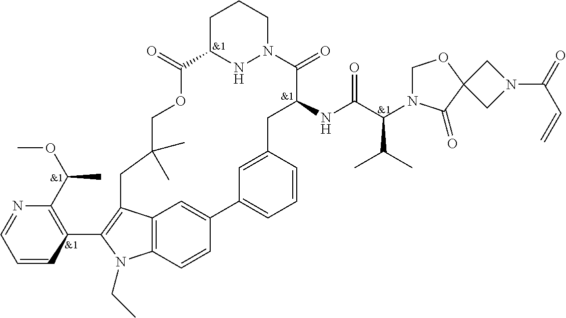 |
| B217 | 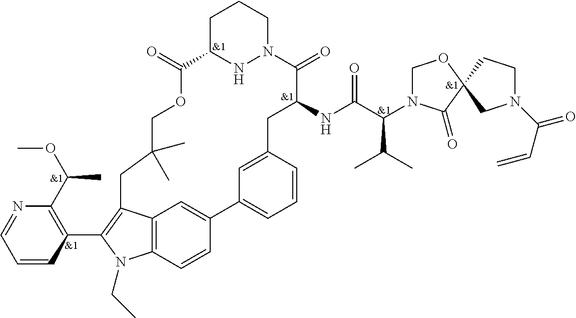 |
| B218 | 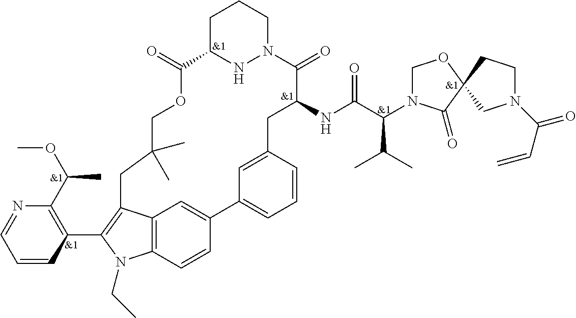 |
| B219 | 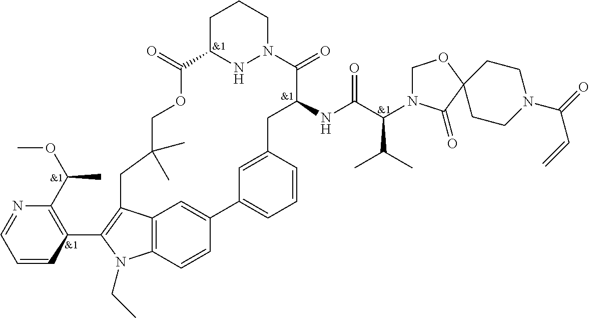 |
| B220 | 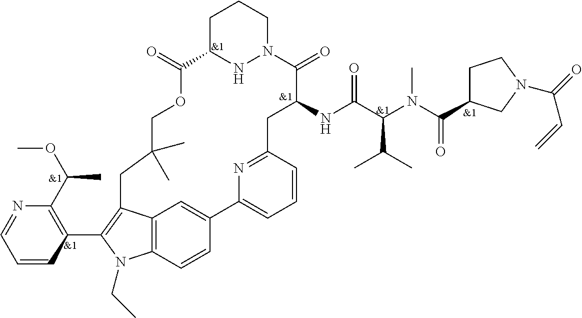 |
| B221 | 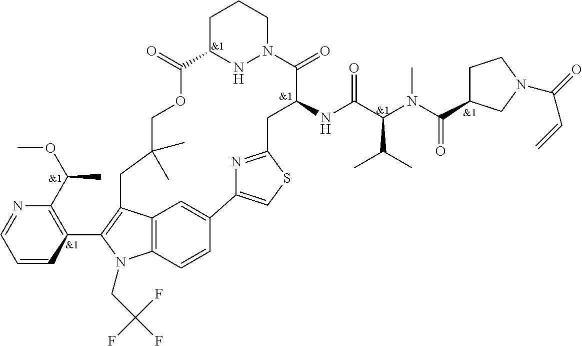 |
| B222 | 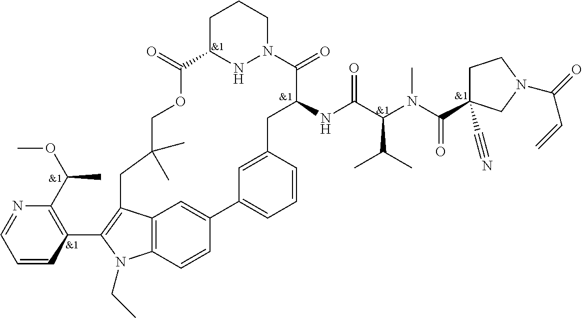 |
| B223 | 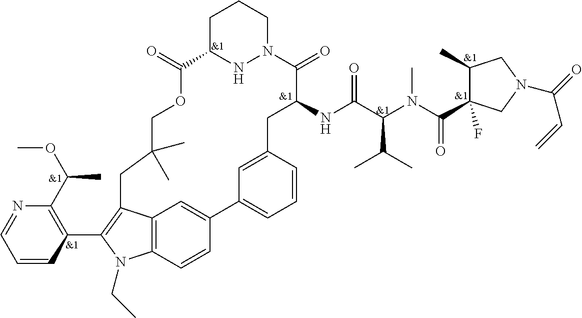 |
| B224 | 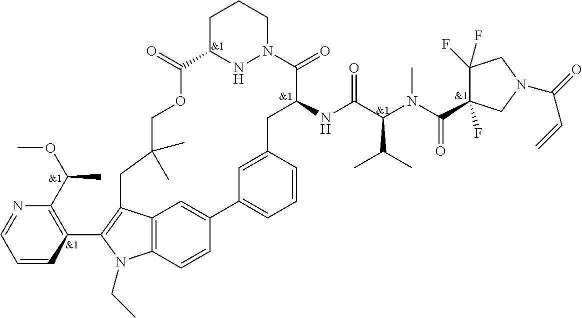 |
| B225 | 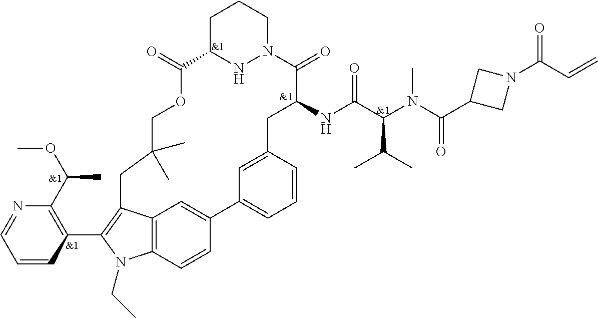 |
| B226 | 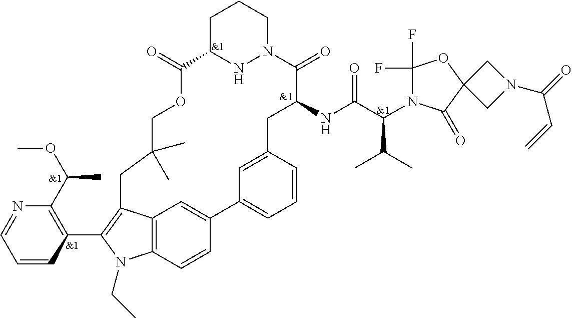 |
| B227 | 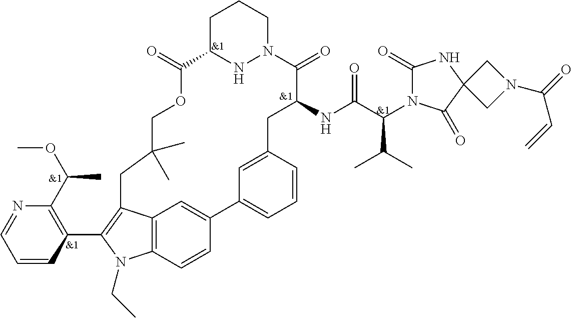 |
| B228 | 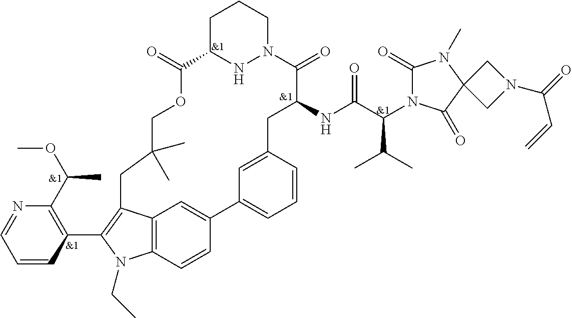 |
| B229 | 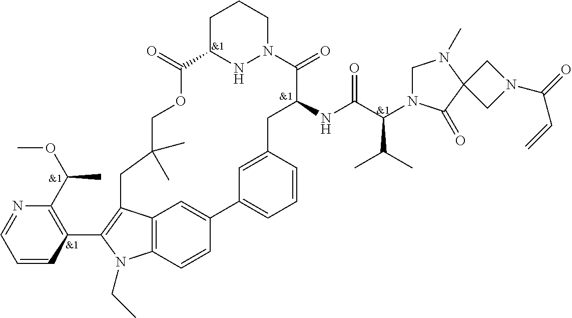 |
| B230 | 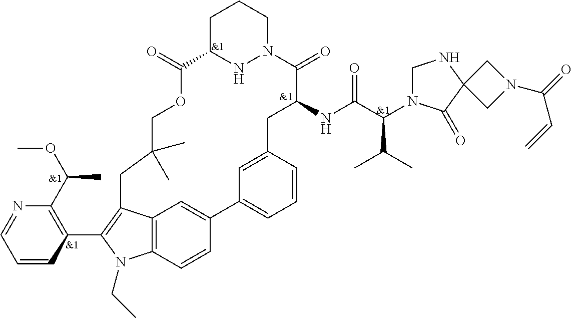 |
| B231 | 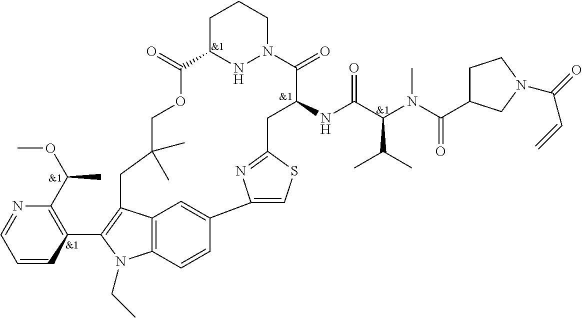 |
| B232 | 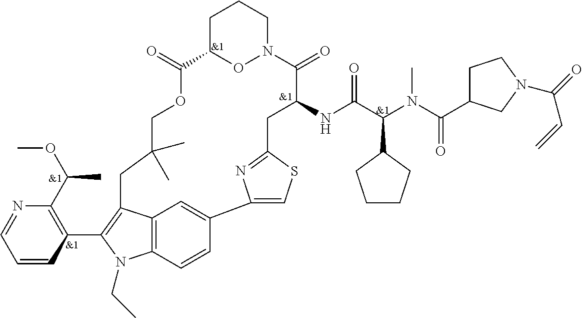 |
| B233 | 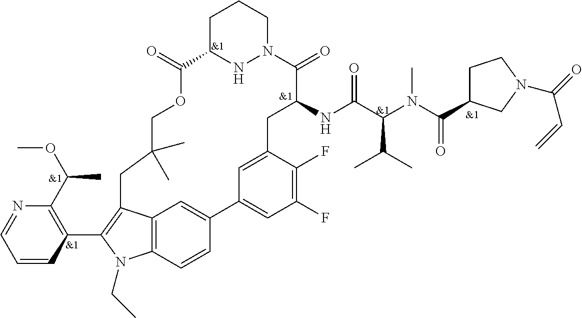 |
| B234 | 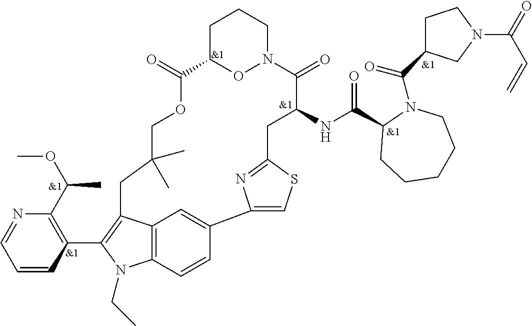 |
| B235 | 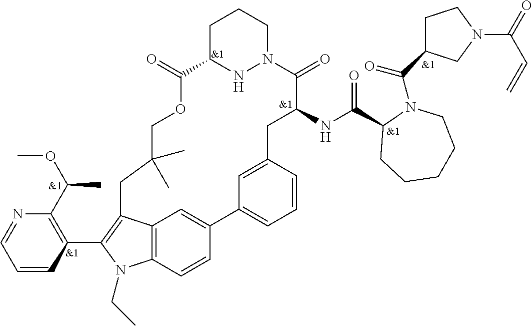 |
| B236 | 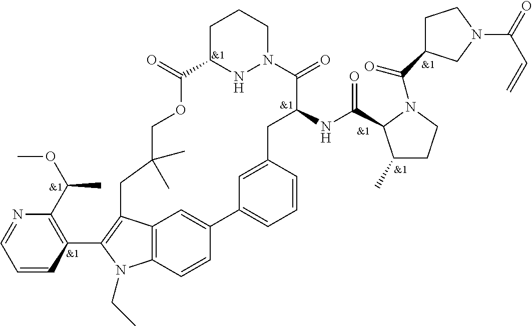 |
| B237 | 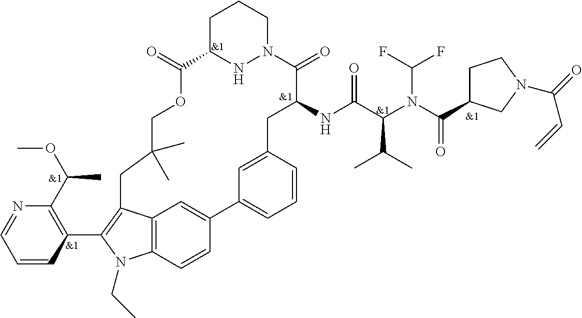 |
| B238 | 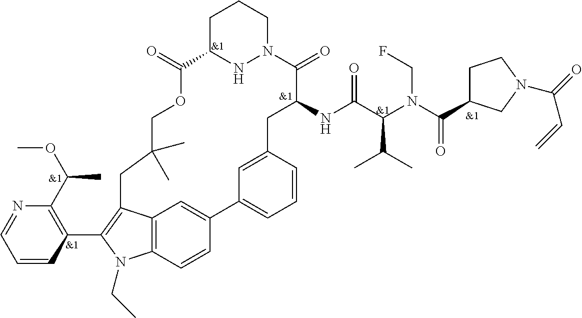 |
| B239 | 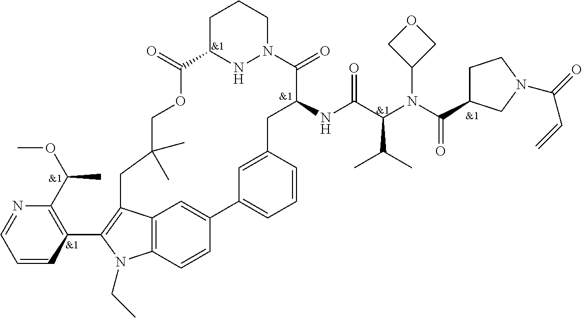 |
| B240 | 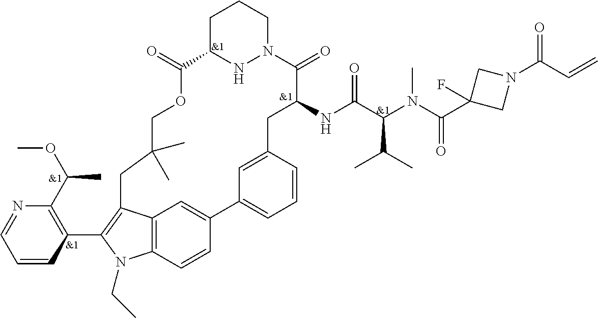 |
| B241 | 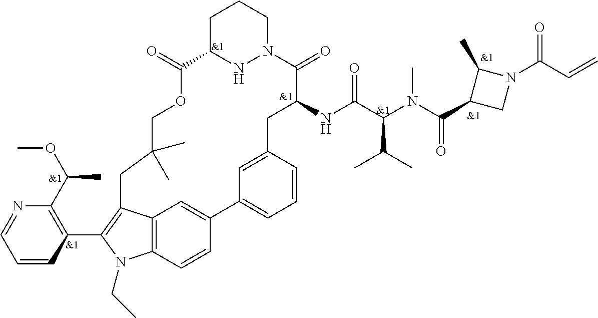 |
| B242 | 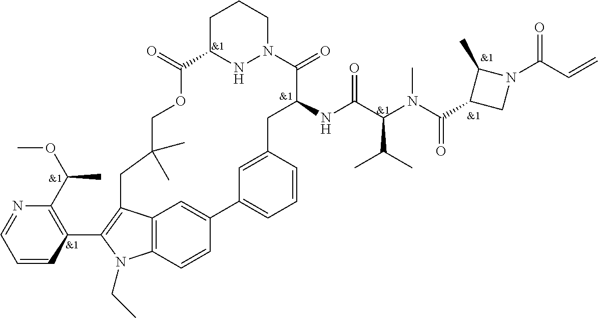 |
| B243 | 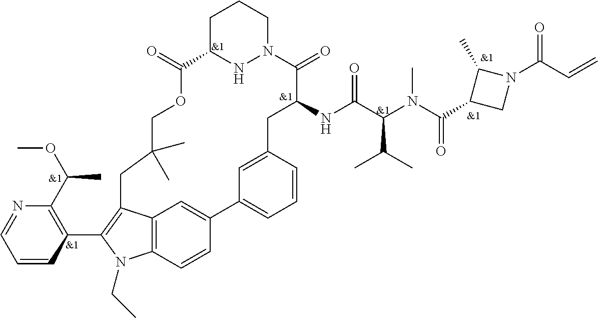 |
| B244 | 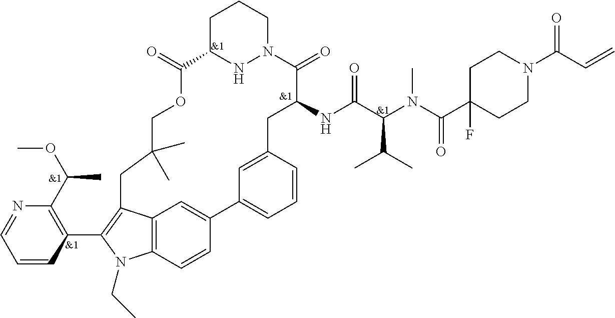 |
| B245 | 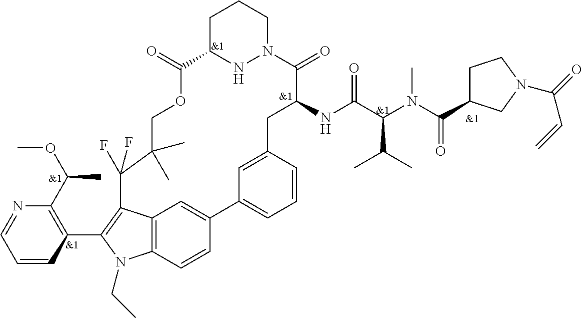 |
| B246 | 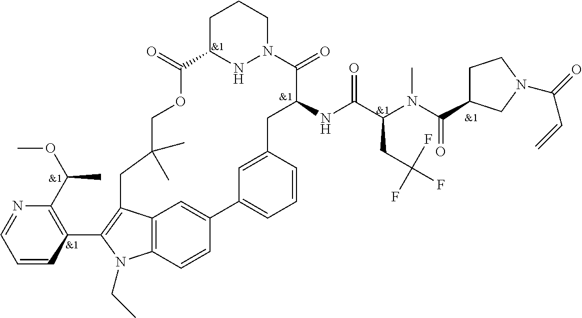 |
| B247 | 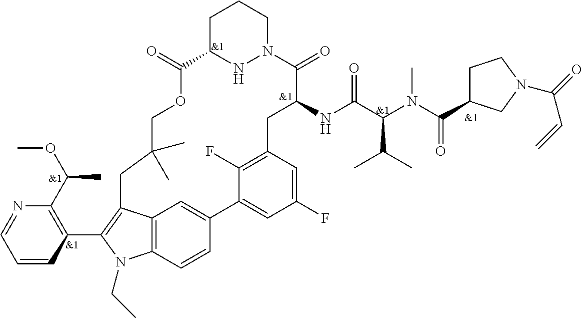 |
| B248 | 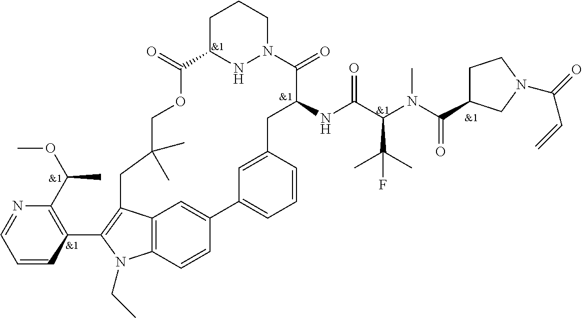 |
| B249 | 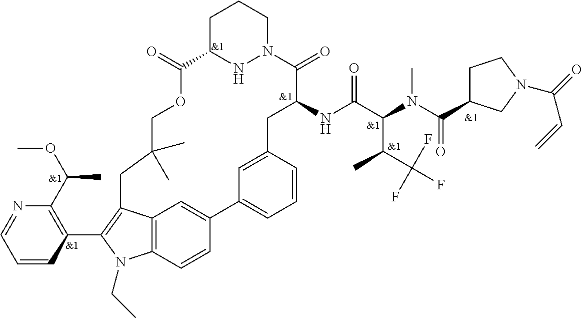 |
| B250 | 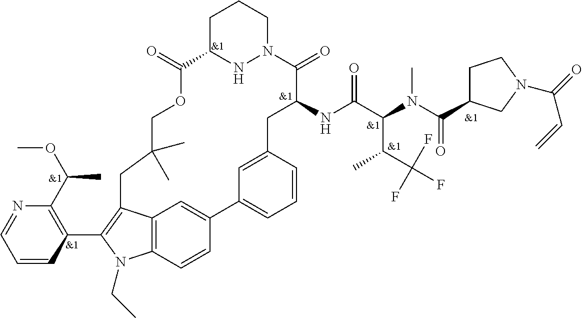 |
| B251 | 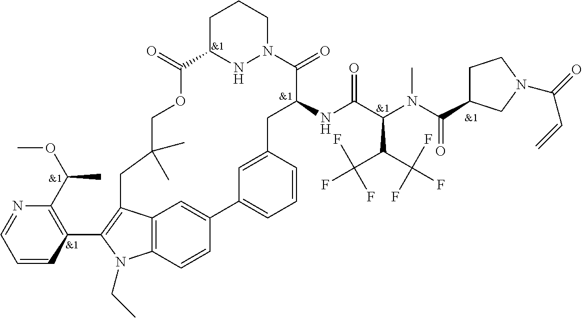 |
| B252 | 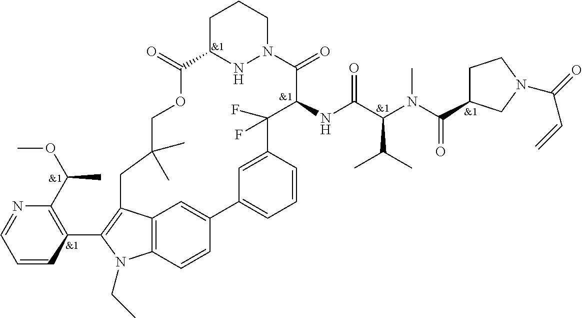 |
| B253 | 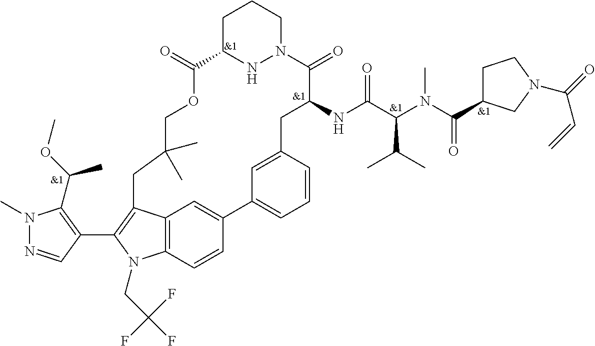 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| B254 | 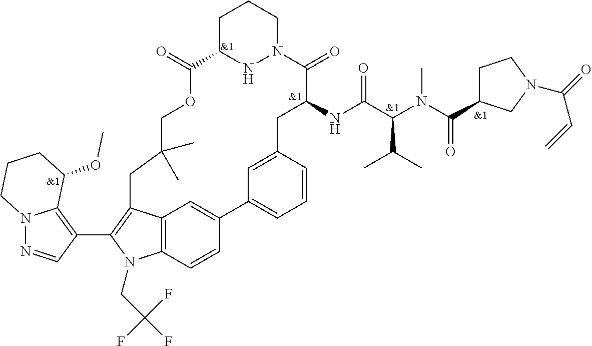 [SMILES: C=CC(=O)N1CC[C@H](C(=O)N(C)[C@H](C(=O)N[C@H]2Cc3cccc(c3)-c3ccc4c(c3)c(c(-c3cnn5c3[C@@H](OC)CCC5)n4CC(F)(F)F)CC(C)(C)COC(=O)[C@@H]3CCCN(N3)C2=O)C(C)C)C1] |
| B255 | 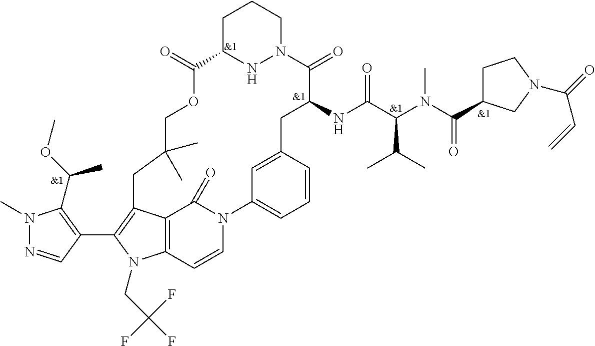 [SMILES: C=CC(=O)N1CC[C@H](C(=O)N(C)[C@H](C(=O)N[C@H]2Cc3cccc(c3)-n3ccc4c(c(c(-c5cnn(C)c5[C@H](C)OC)n4CC(F)(F)F)CC(C)(C)COC(=O)[C@@H]4CCCN(N4)C2=O)c3=O)C(C)C)C1] |
| B256 | 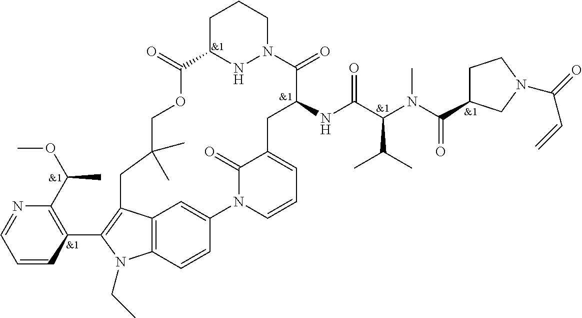 |
| B257 | 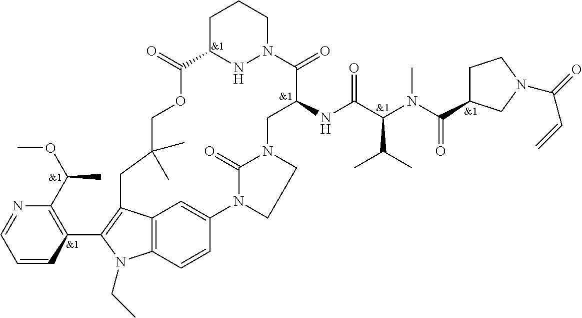 |
| B258 | 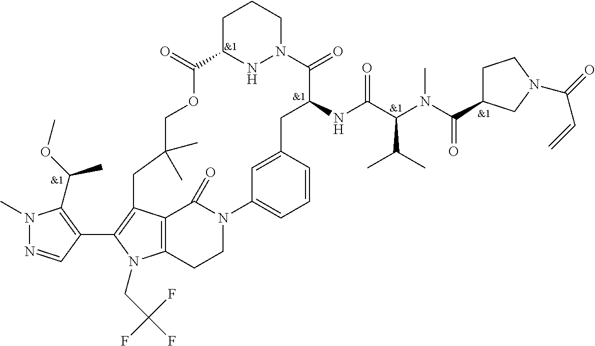 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3=O)C(C)C)C1] |
| B259 | 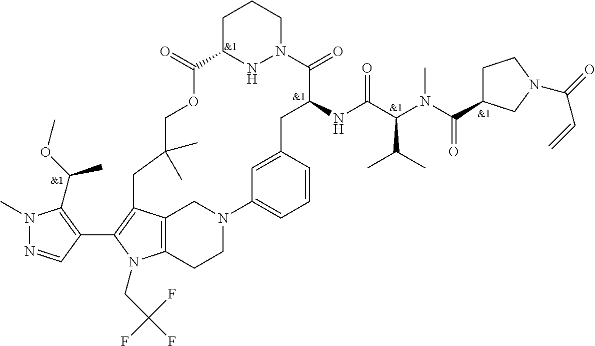 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| B260 | 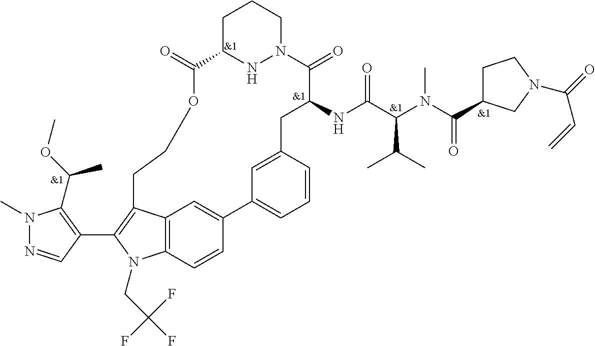 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CCOC(=O)[C@@H]3CCCN(N3)C2=O)C(C)C)C1] |
| B261 | 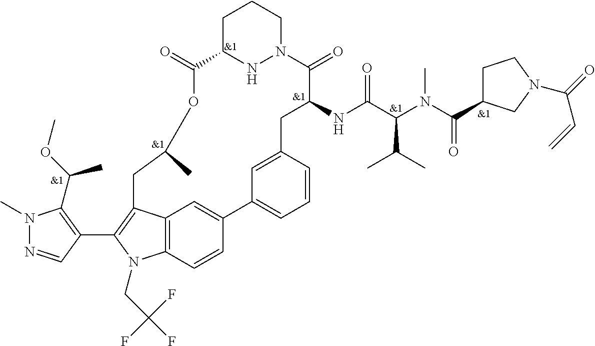 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@H](C)OC(=O)[C@@H]3CCCN(N3)C2=O)C(C)C)C1] |
| B262 | 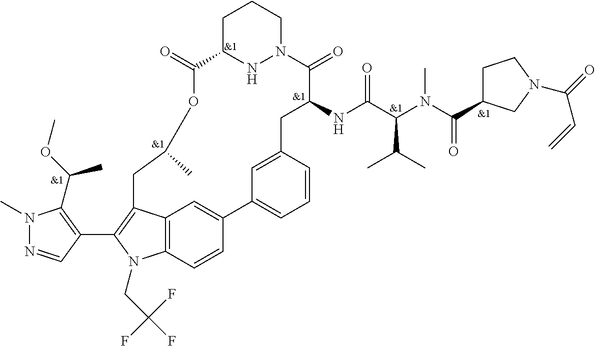 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@@H](C)OC(=O)[C@@H]3CCCN(N3)C2=O)C(C)C)C1] |
| B263 | 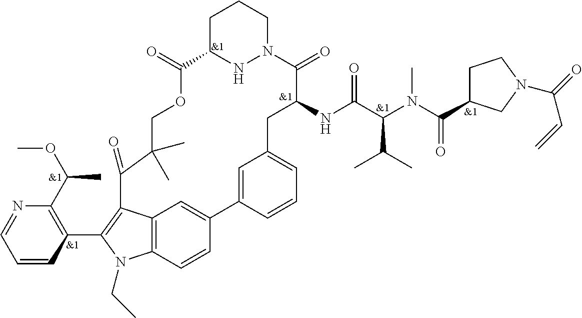 |
| B264 | 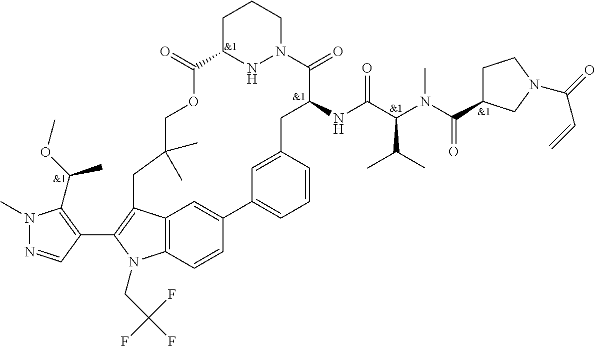 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| B265 | 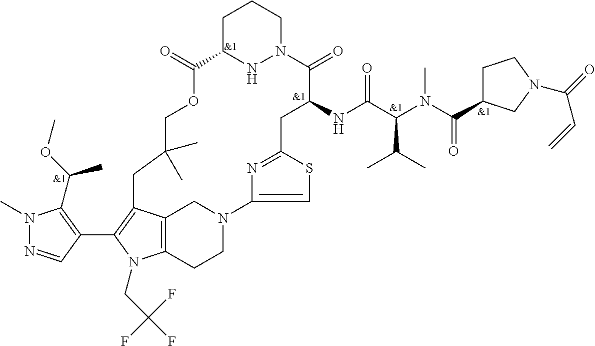 [SMILES: C=CC(=O)N1CC[C@H](C(=O)N(C)[C@H](C(=O)N[C@H]2Cc3nc(cs3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| B266 | 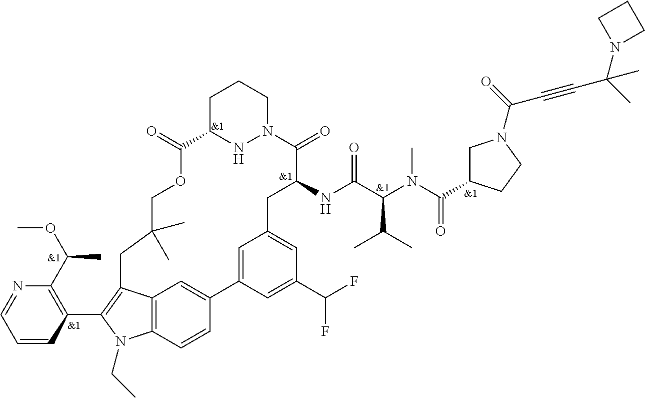 |
| B267 | 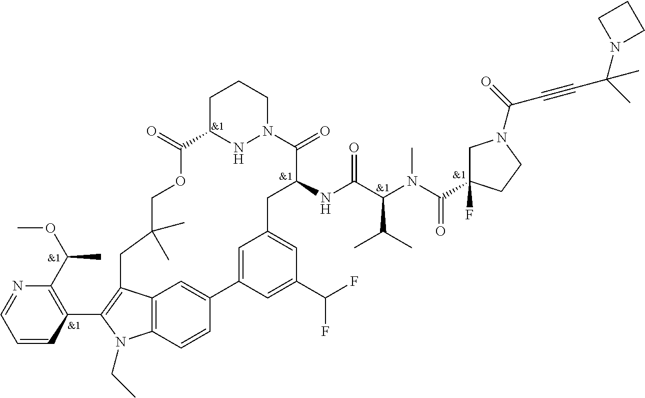 |
| B268 | 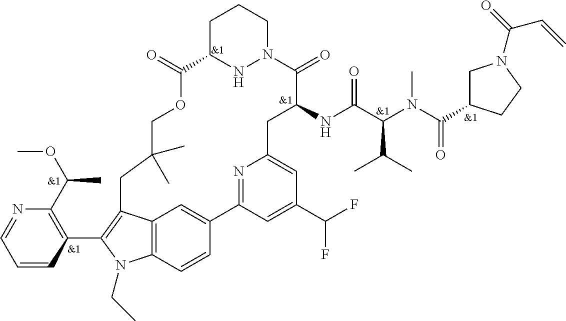 |
| B269 | 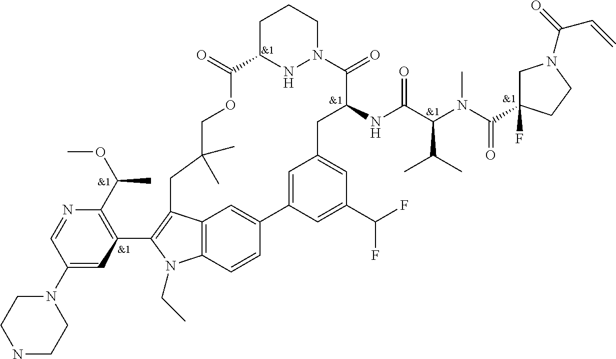 |
| B270 | 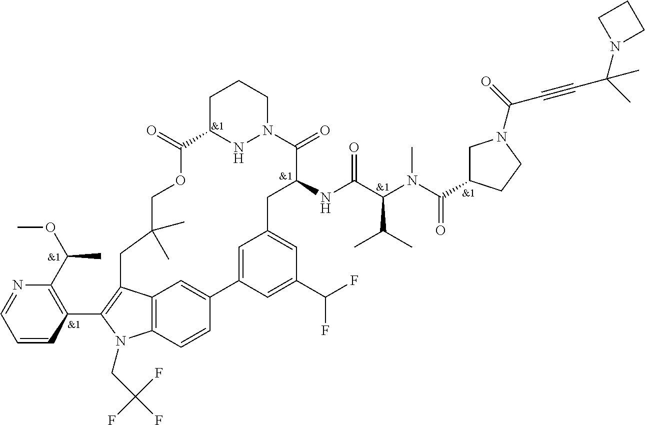 |
| B271 | 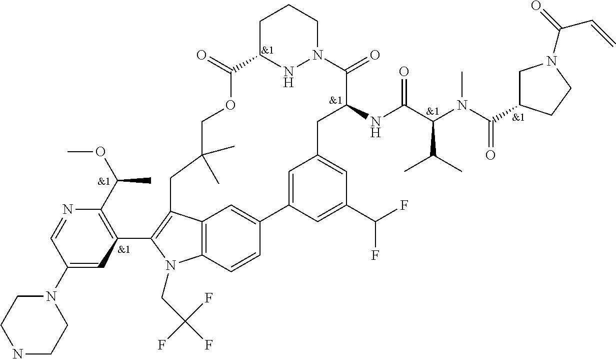 |
| B272 | 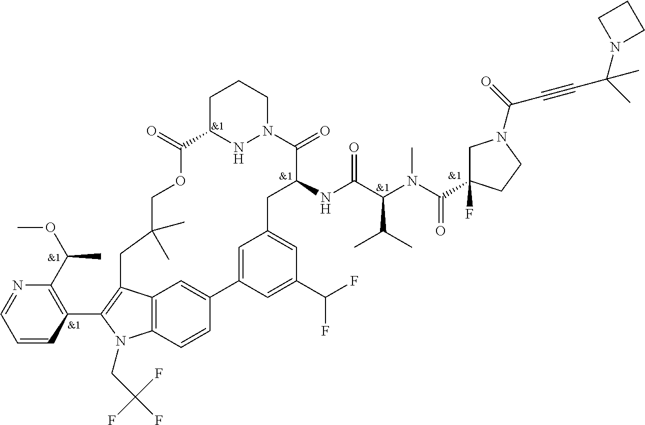 |
| B273 | 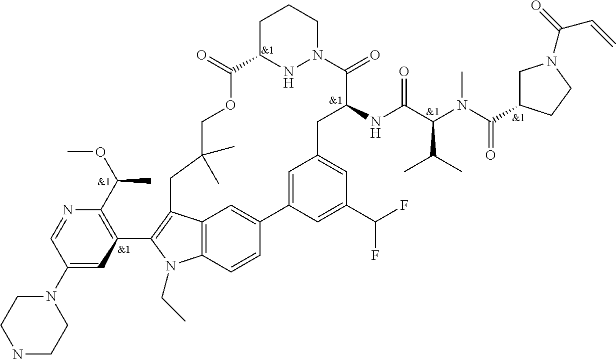 |
| B274 | 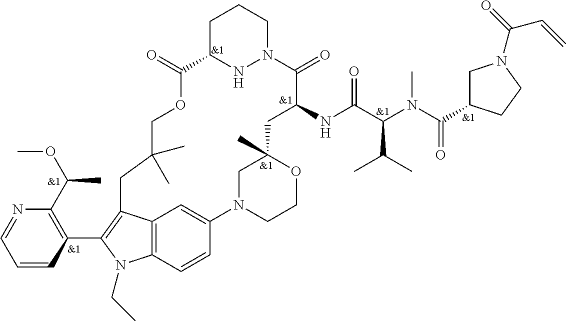 |
| B275 | 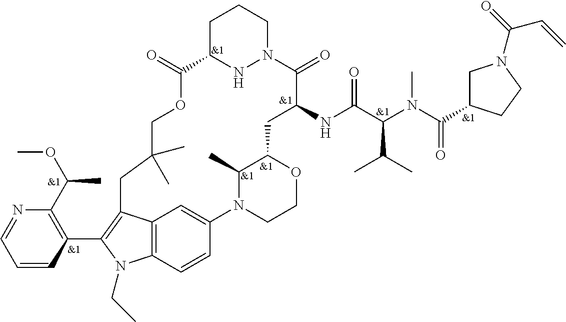 |
| B276 | 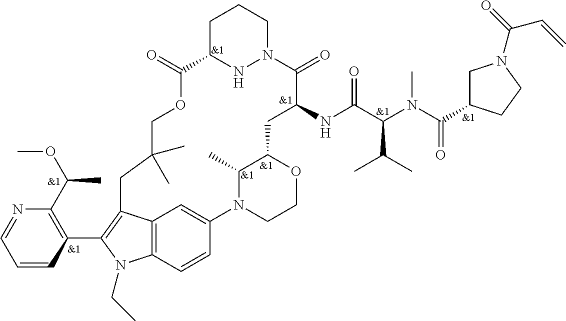 |
| B277 | 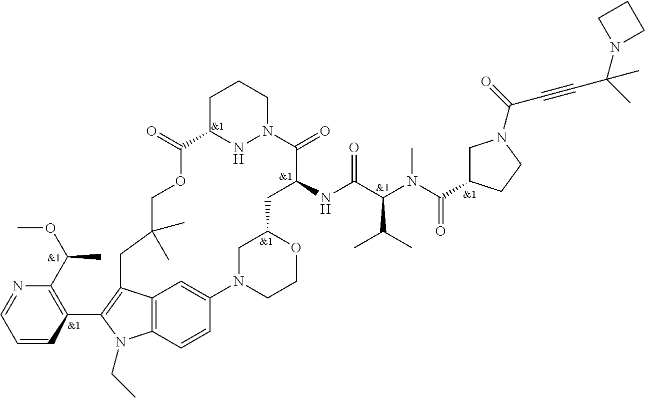 |
| B278 | 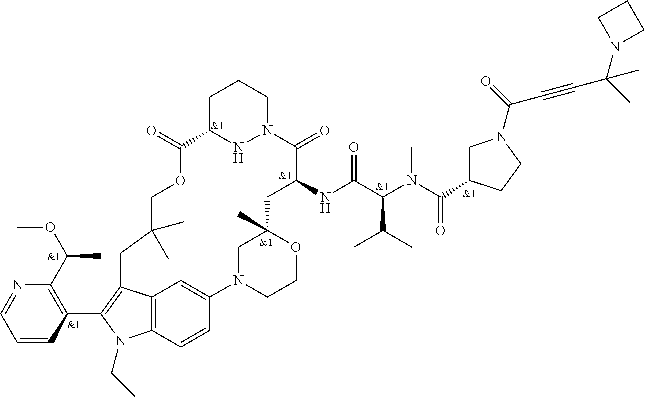 |
| B279 | 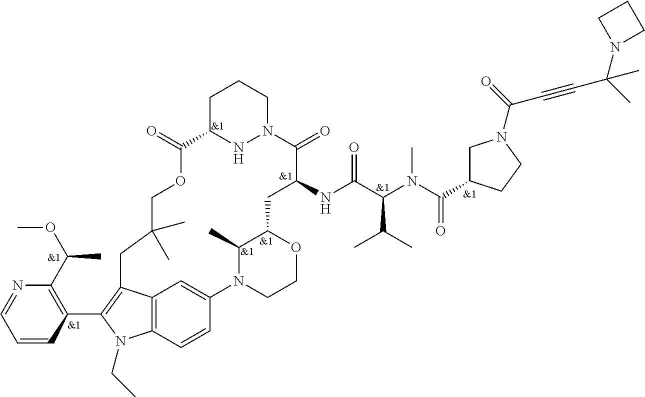 |
| B280 | 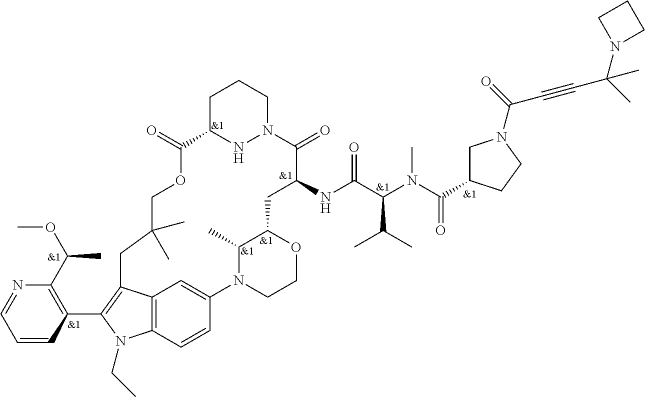 |
| B282 | 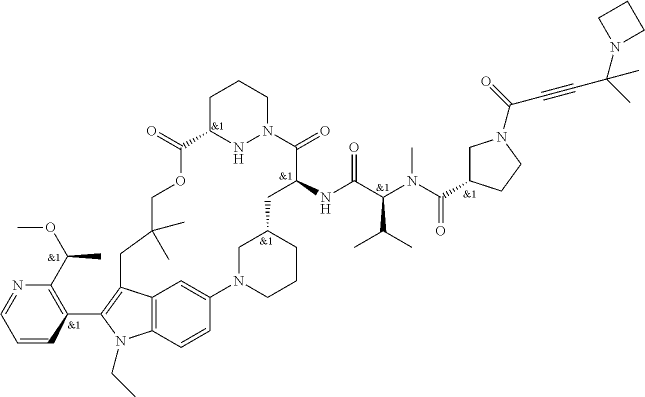 |
| B283 | 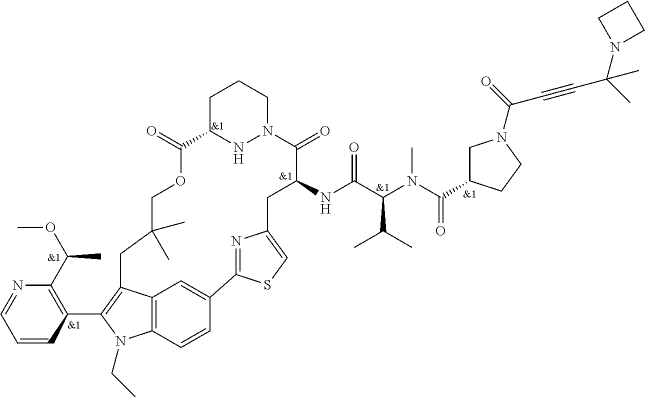 |
| B284 | 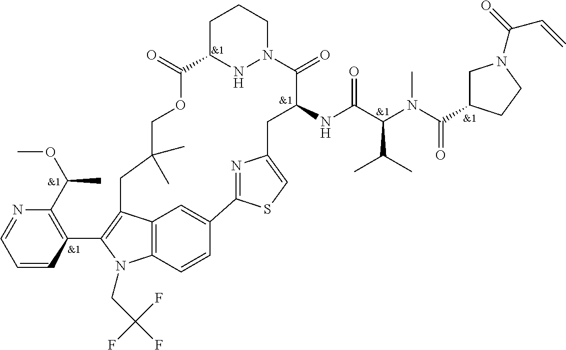 |
| B285 | 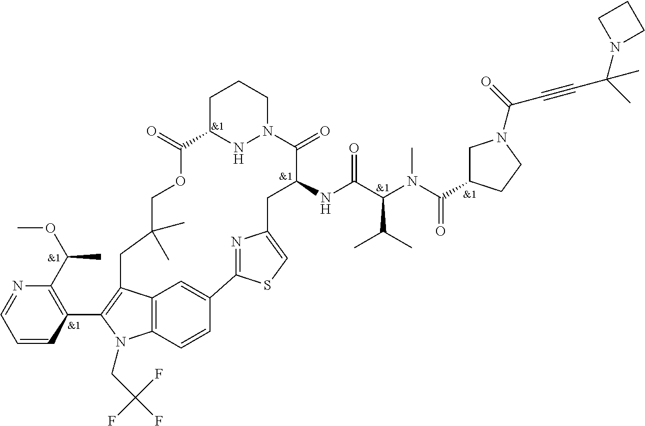 |
| B286 | 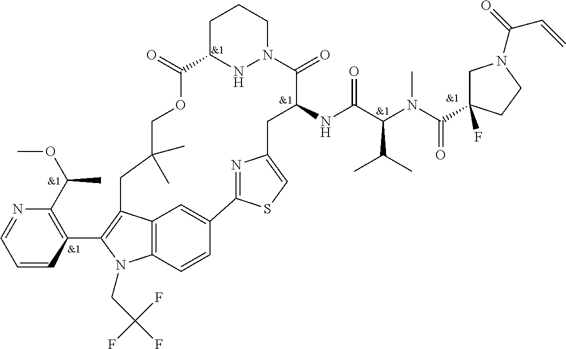 |
| B287 | 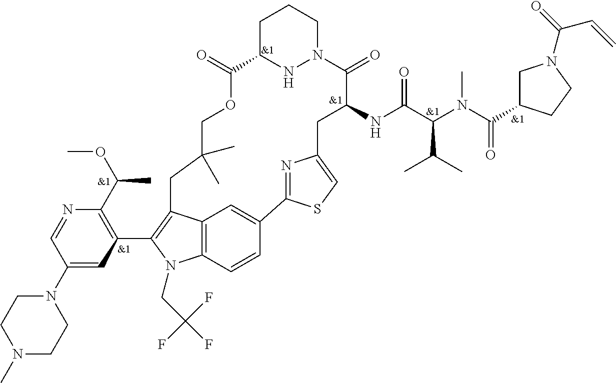 |
| B288 | 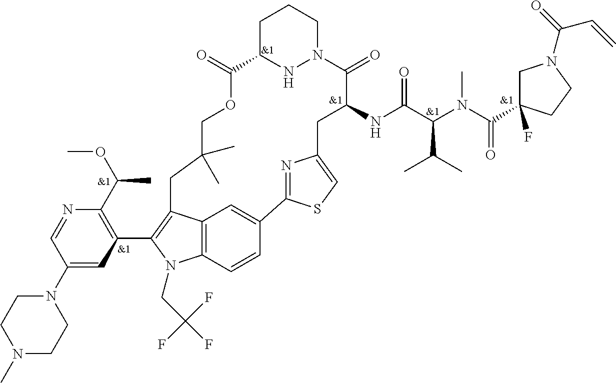 |
| B289 | 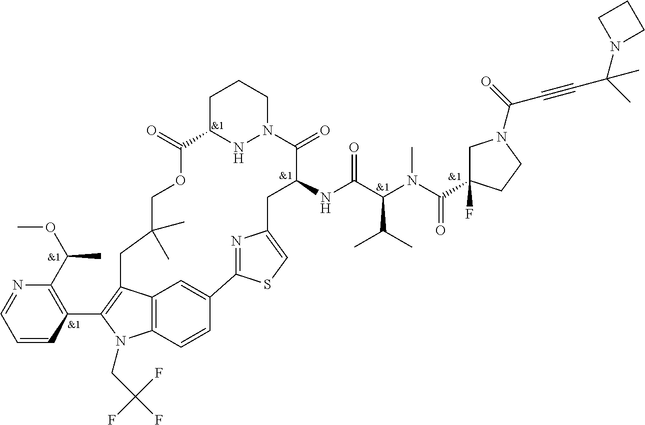 |
| B290 | 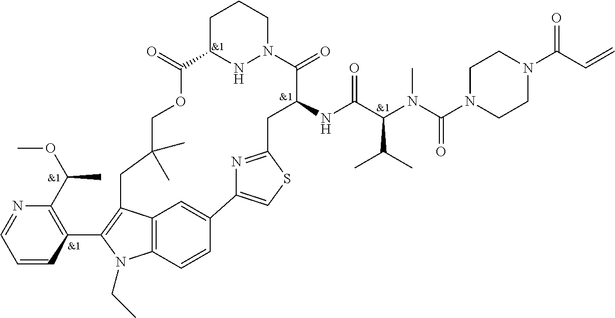 |
| B291 | 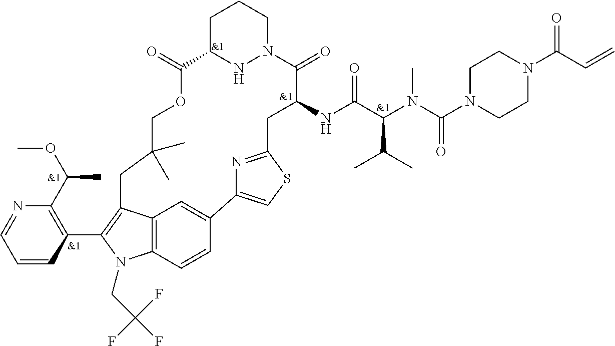 |
| B292 | 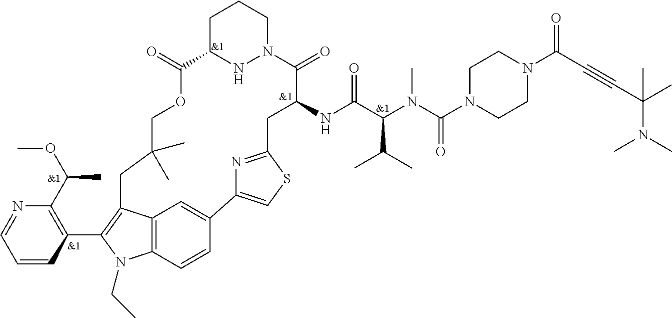 |
| B293 | 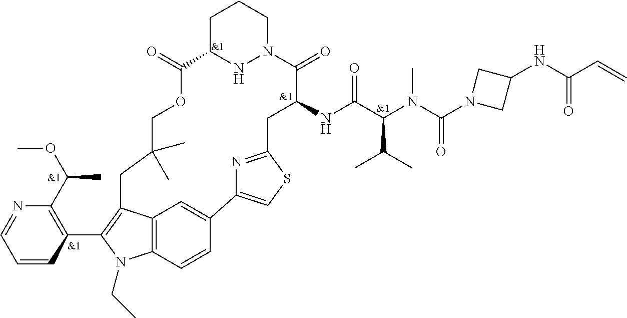 |
| B294 | 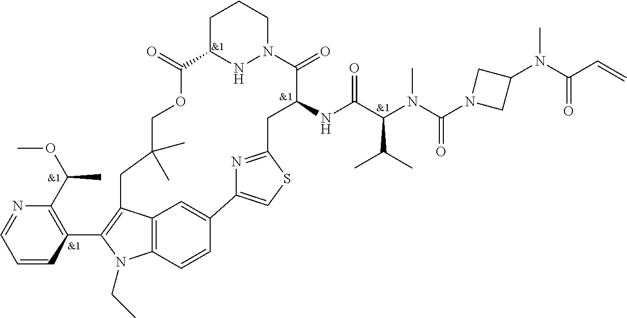 |
| B295 | 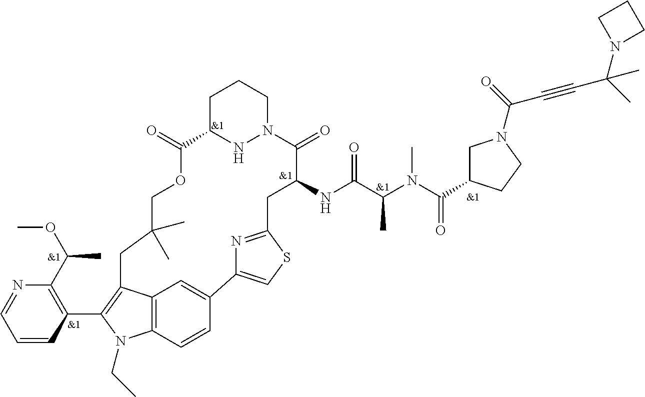 |
| B296 | 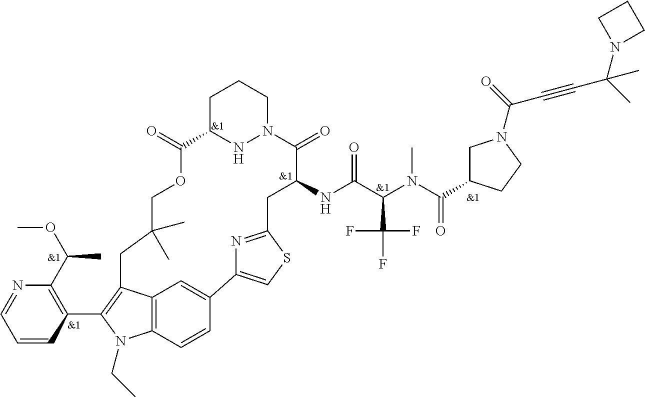 |
| B297 | 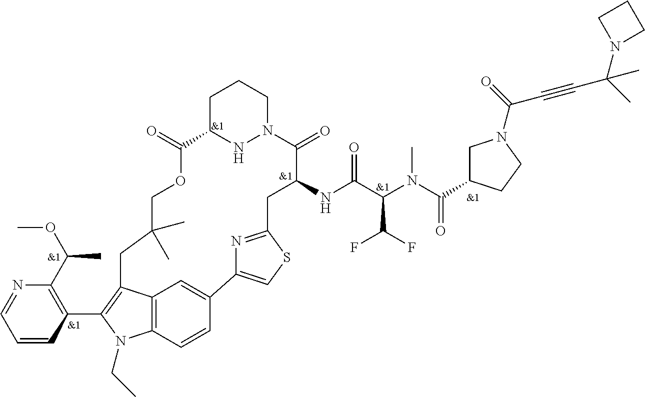 |
| B298 | 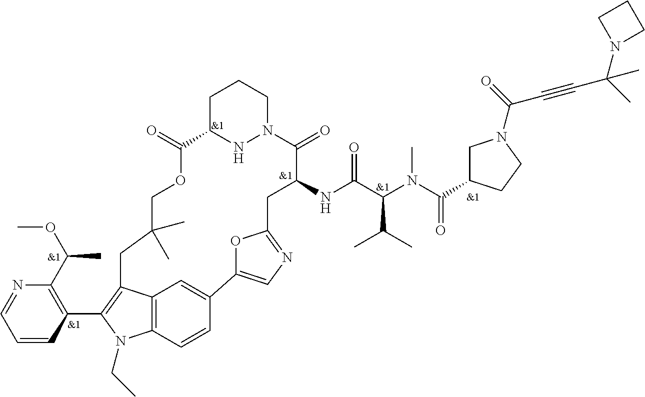 |
| B299 | 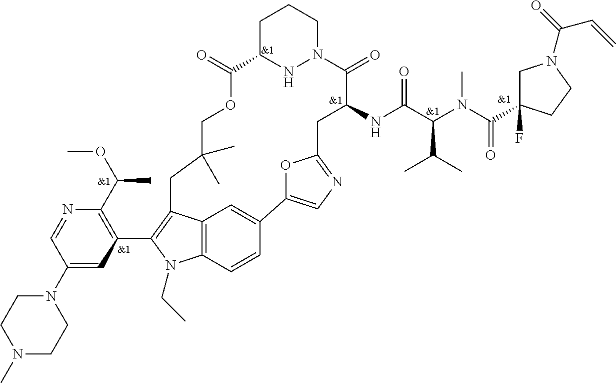 |
| B300 | 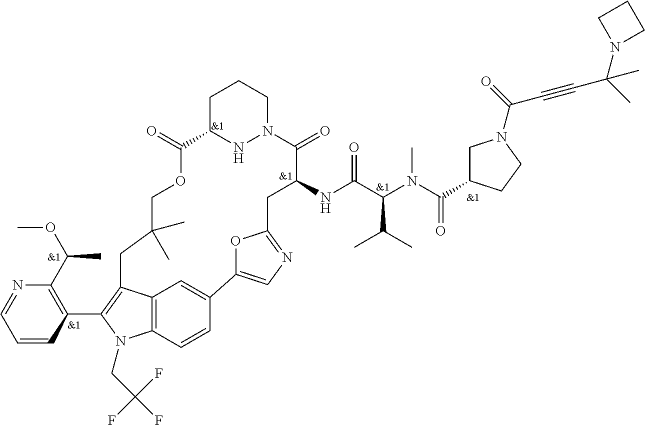 |
| B301 | 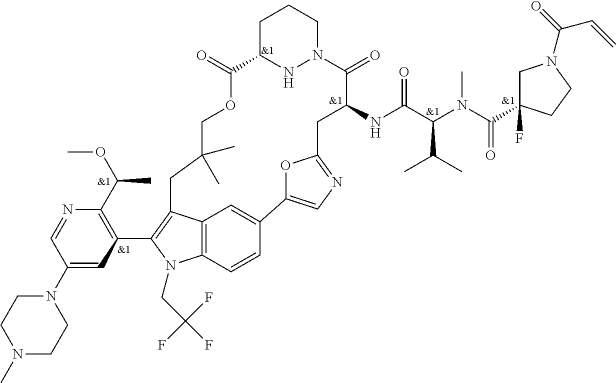 |
| B302 | 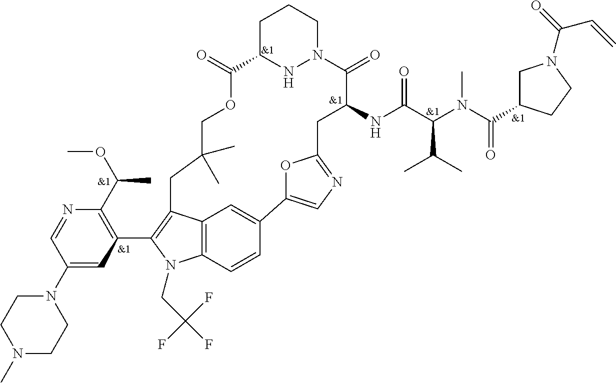 |
| B303 | 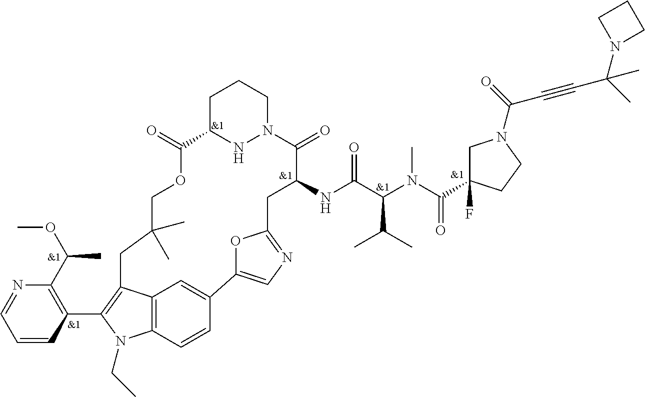 |
| B304 | 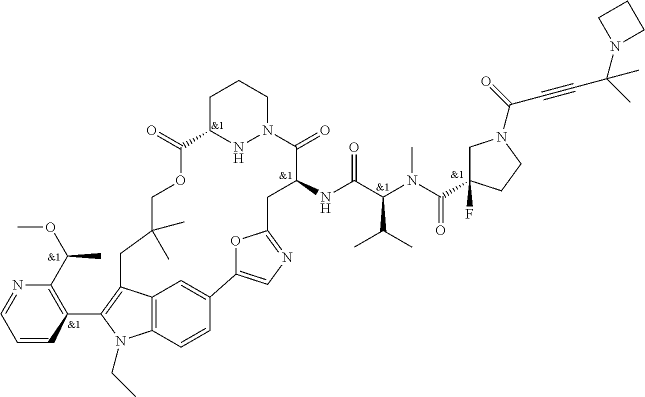 |
| B305 | 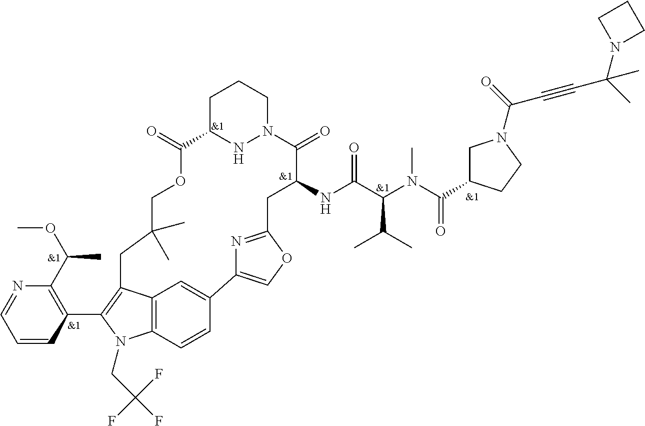 |
| B306 | 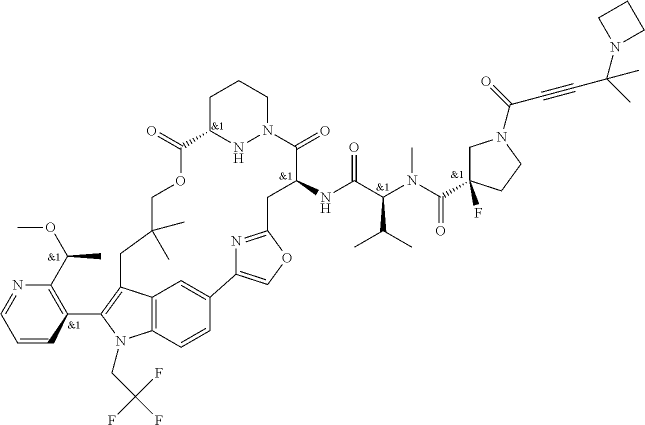 |
| B307 | 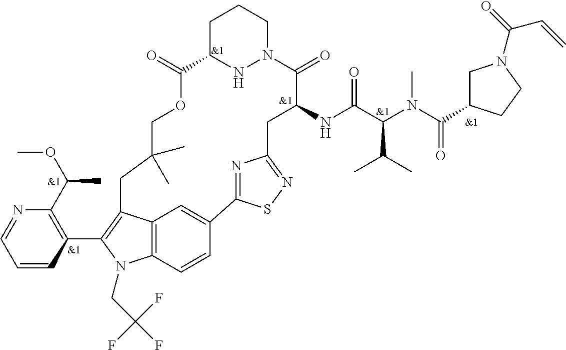 |
| B308 | 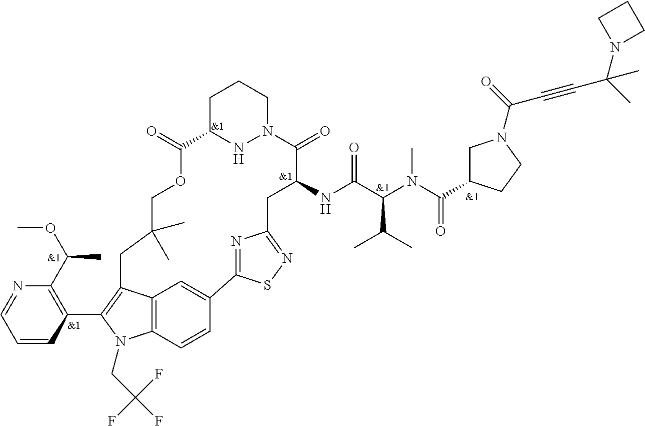 |
| B309 | 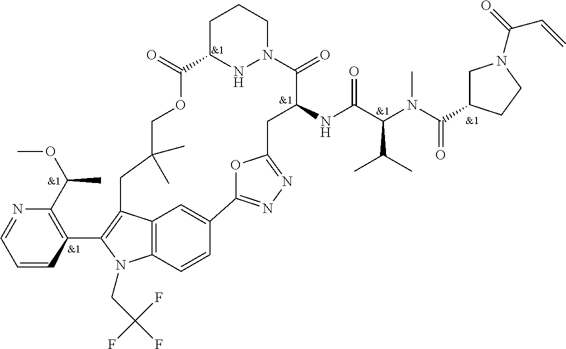 |
| B310 | 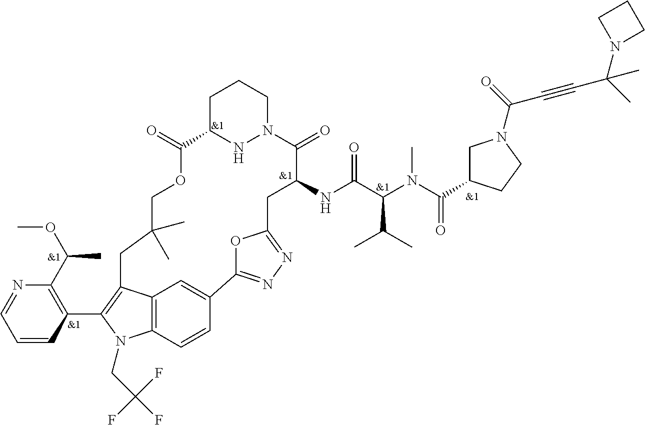 |
| B311 | 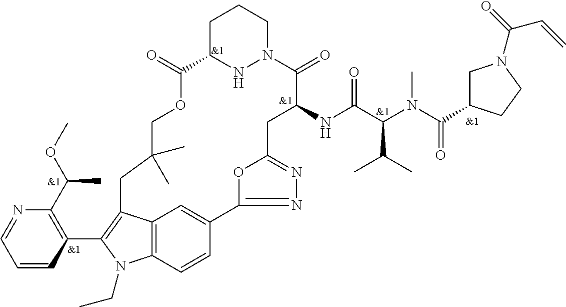 |
| B312 | 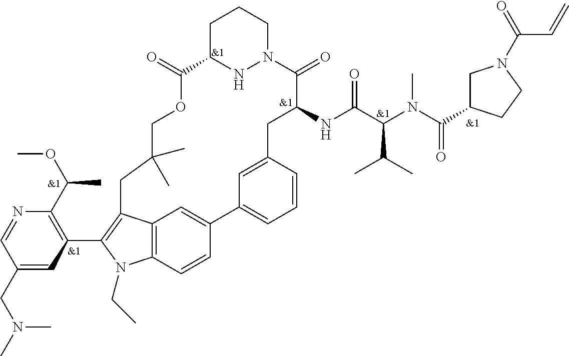 |
| B313 | 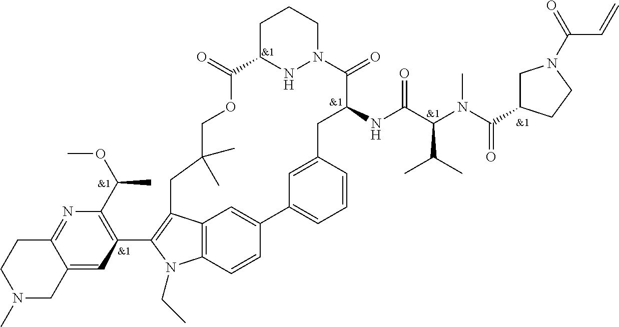 |
| B314 | 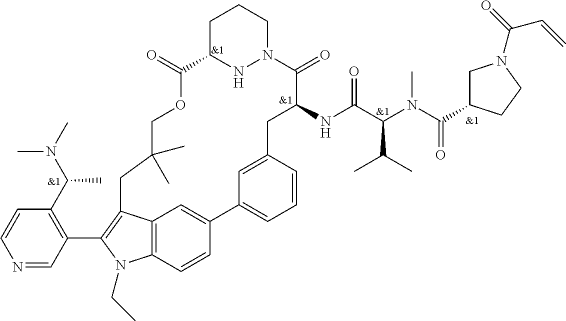 [SMILES: C=CC(=O)N1CC[C@H](C(=O)N(C)[C@H](C(=O)N[C@H]2Cc3cccc(c3)-c3ccc4c(c3)c(c(-c3cnccc3[C@@H](C)N(C)C)n4CC)CC(C)(C)COC(=O)[C@@H]3CCCN(N3)C2=O)C(C)C)C1] |
| B315 | 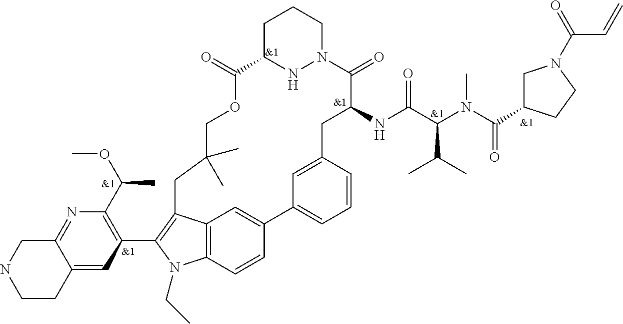 |
| B316 | 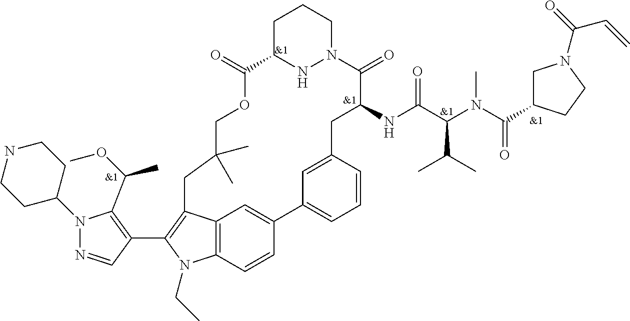 [SMILES: C=CC(=O)N1CC[C@H](C(=O)N(C)[C@H](C(=O)N[C@H]2Cc3cccc(c3)-c3ccc4c(c3)c(c(-c3cnn(C5CCNCC5)c3[C@H](C)OC)n4CC)CC(C)(C)COC(=O)[C@@H]3CCCN(N3)C2=O)C(C)C)C1] |
| B317 | 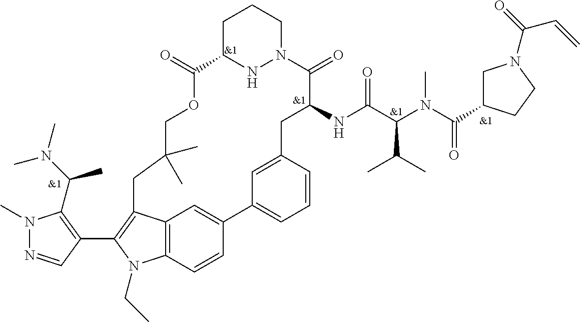 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)N(C)C)n4CC)CC(C)(C)COC(=O)[C@@H]3CCCN(N3)C2=O)C(C)C)C1] |
| B318 | 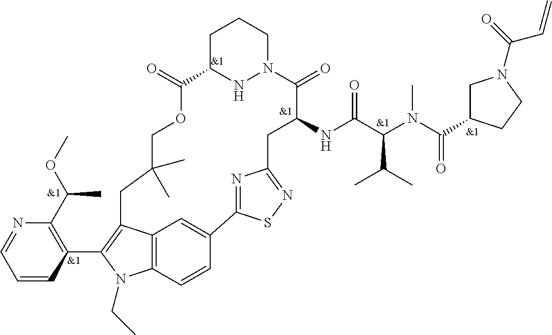 |
| B319 | 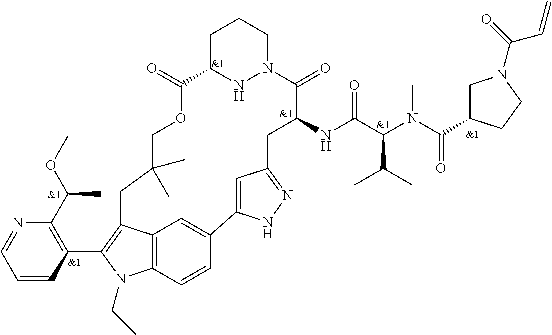 |
| B320 | 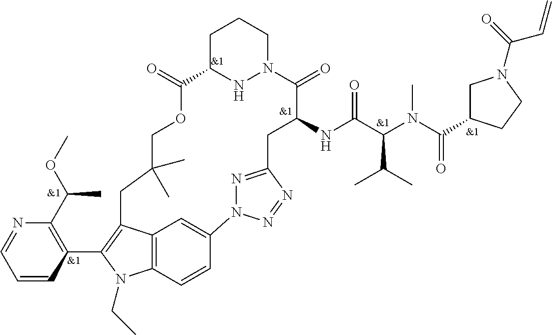 |
| B321 | 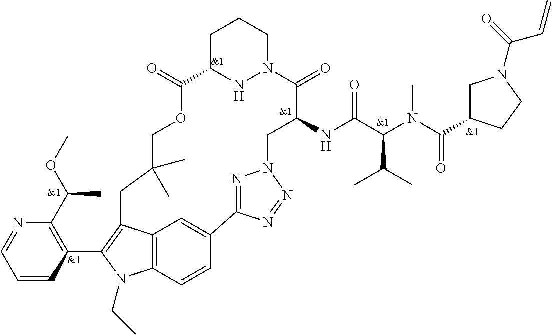 |
| B322 | 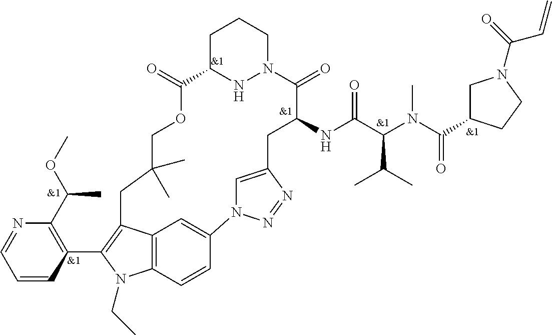 |
| B323 | 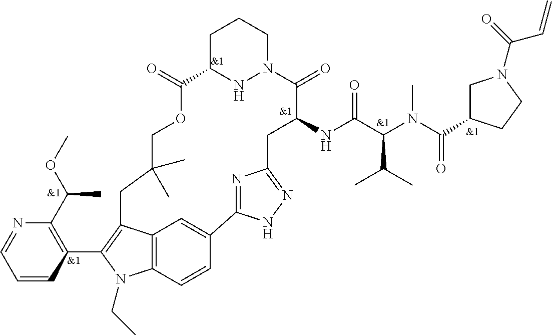 |
| B324 | 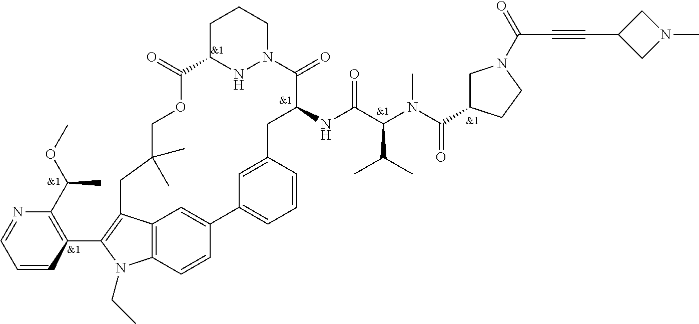 |
| B325 | 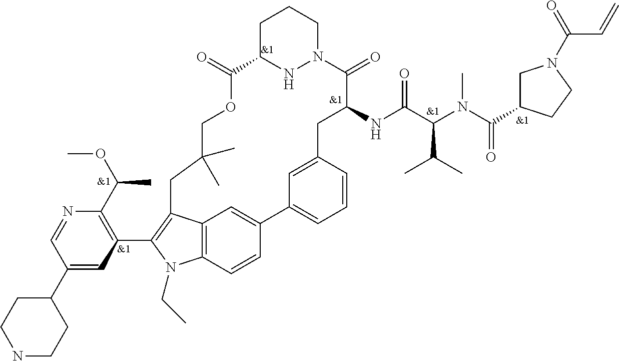 |
| B326 | 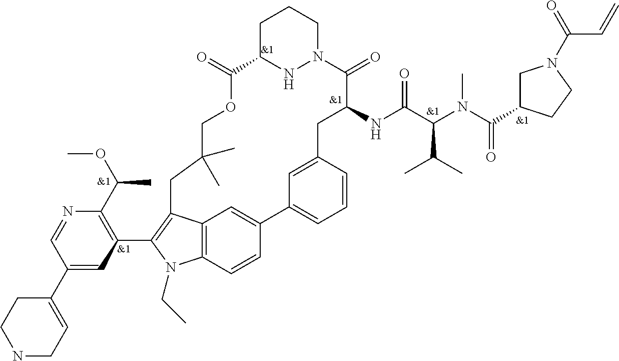 |
| B327 | 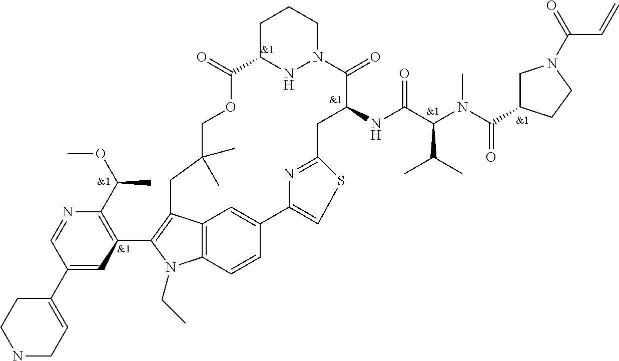 |
| B328 | 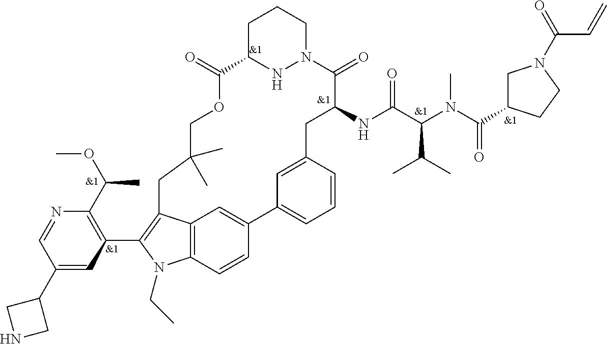 |
| B329 | 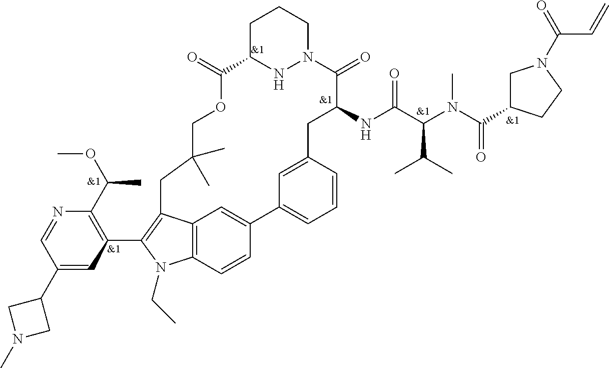 |
| B330 | 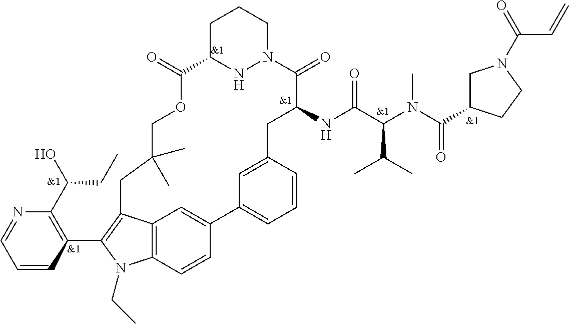 |
| B331 | 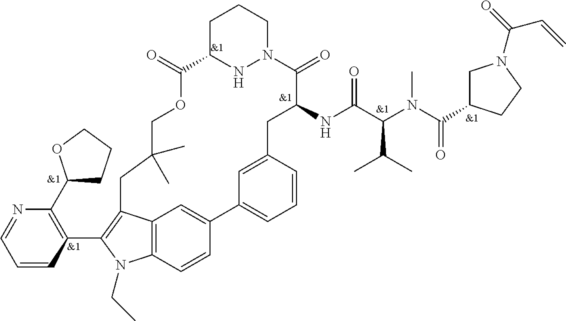 |
| B332 | 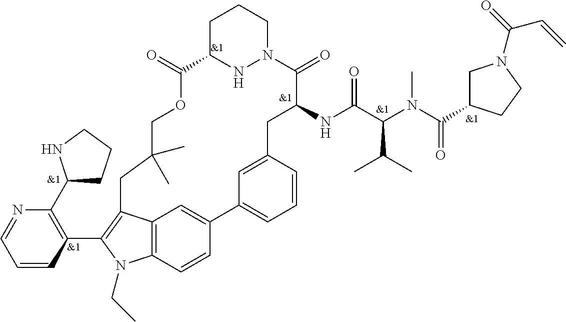 |
| B333 | 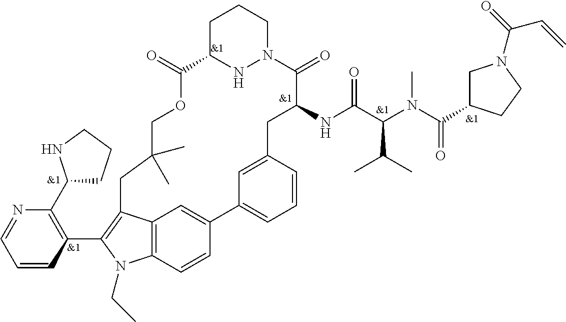 |
| B334 | 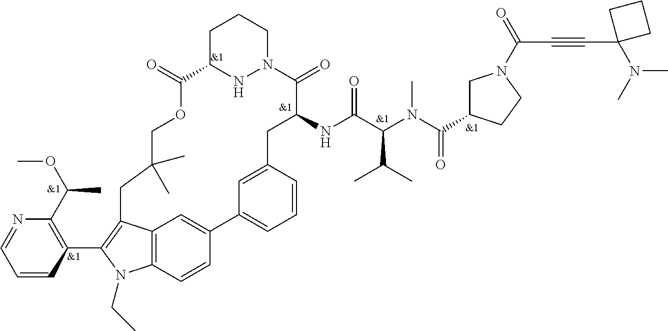 |
| B335 | 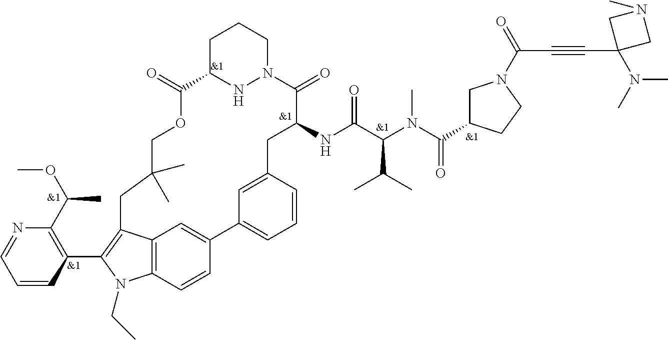 |
| B336 | 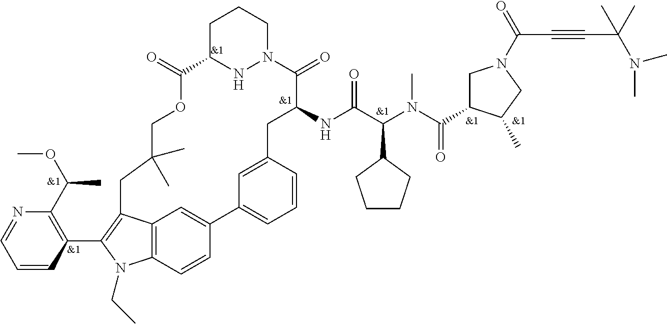 |
| B337 | 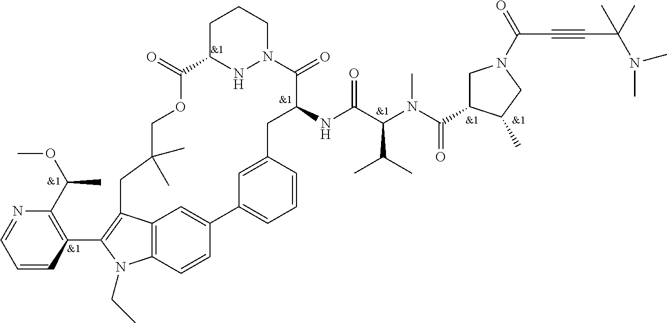 |
| B338 | 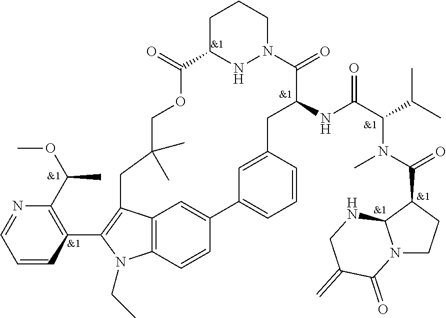 |
| B339 | 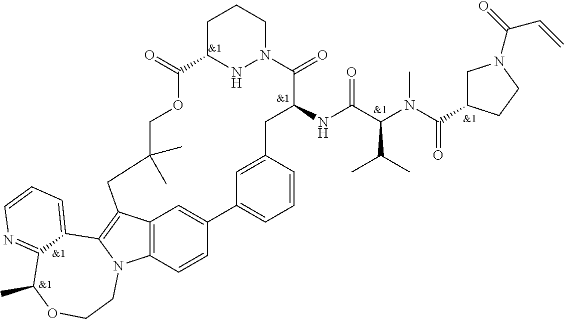 |
| B340 | 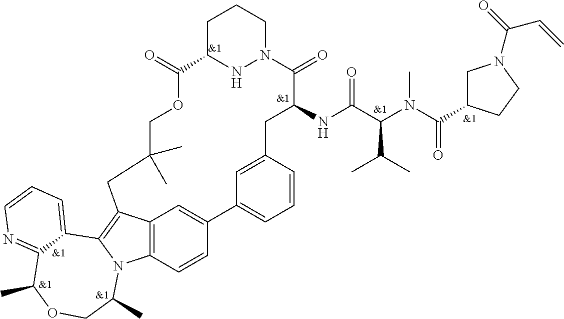 |
| B341 | 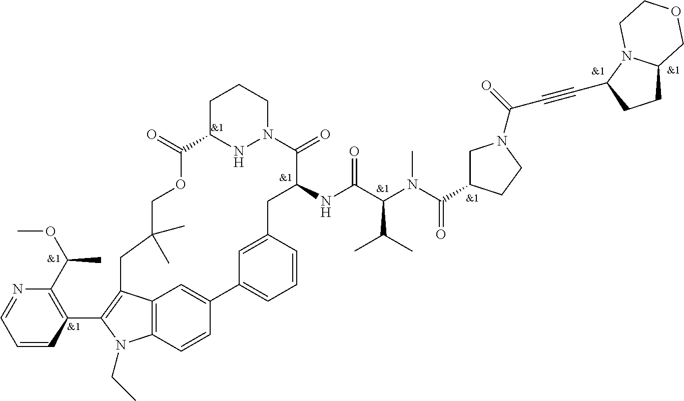 |
| B342 | 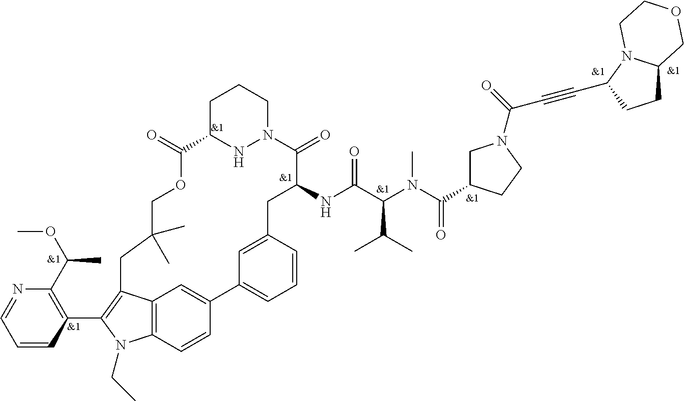 |
| B343 | 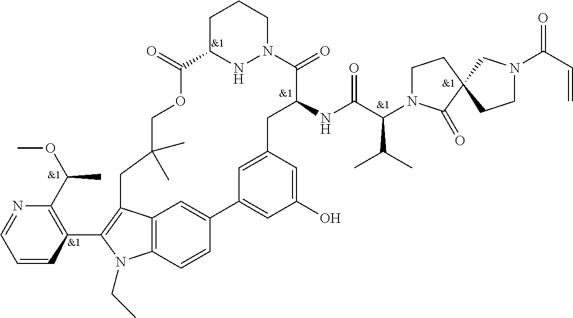 |
| B344 | 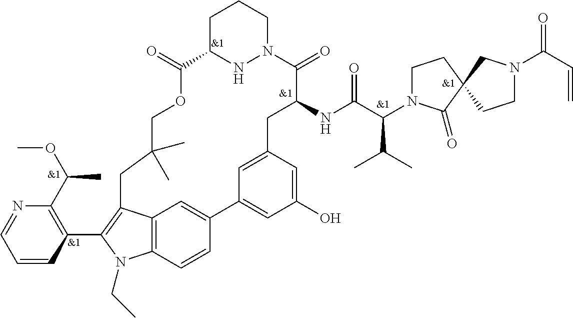 |
| B345 | 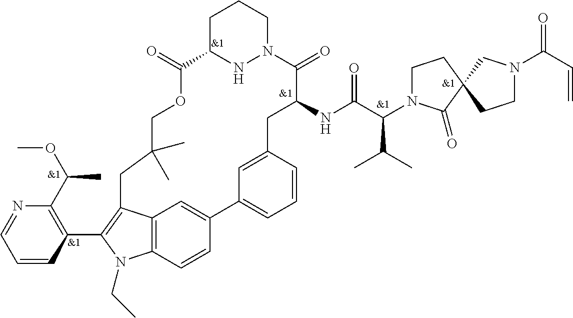 |
| B346 | 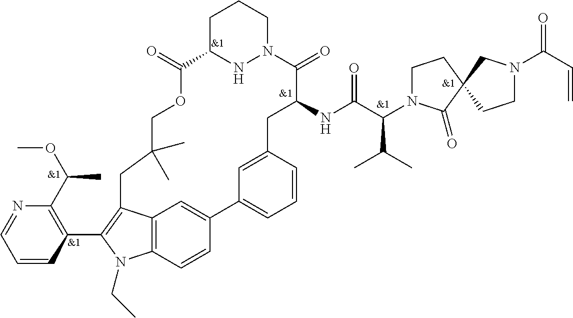 |
| B347 | 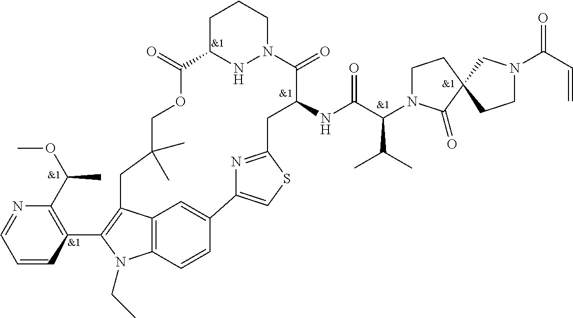 |
| B348 | 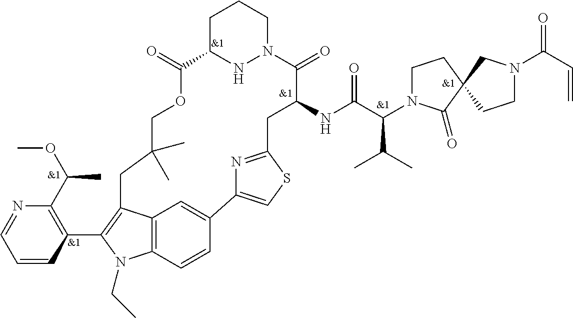 |
| B349 | 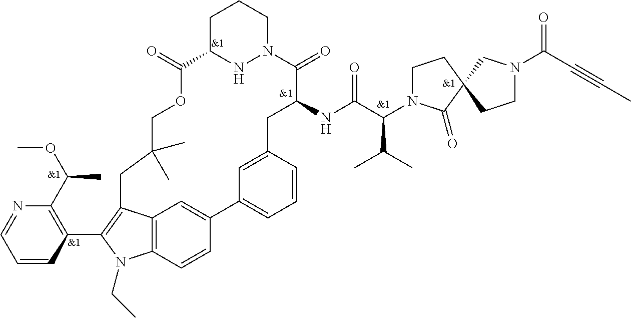 |
| B350 | 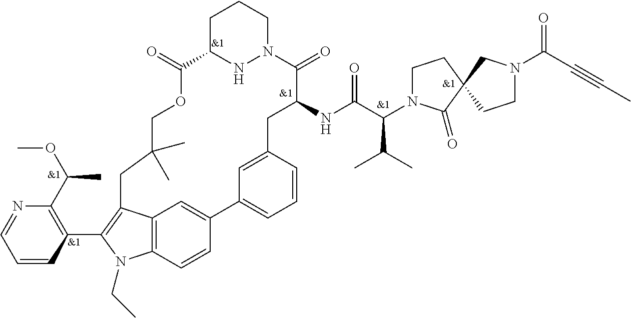 |
| B351 | 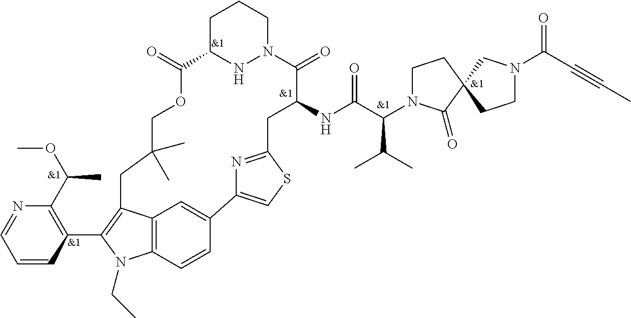 |
| B352 | 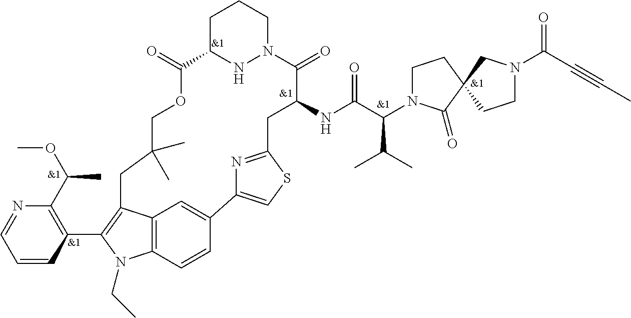 |
| B353 | 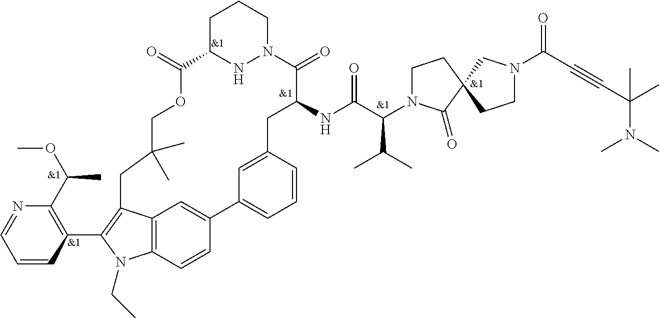 |
| B354 | 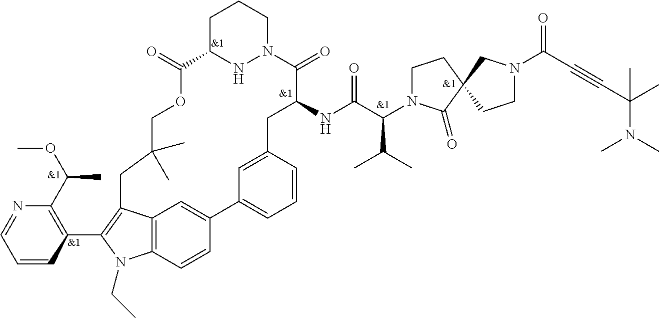 |
| B355 | 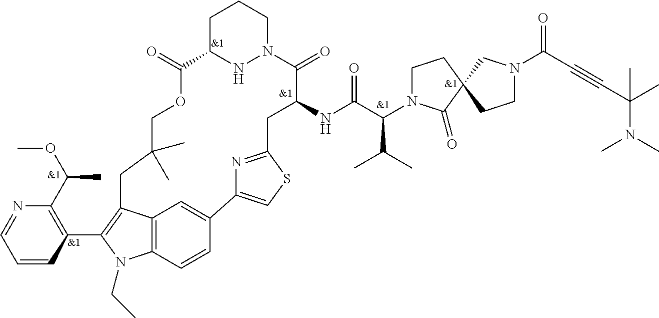 |
| B356 | 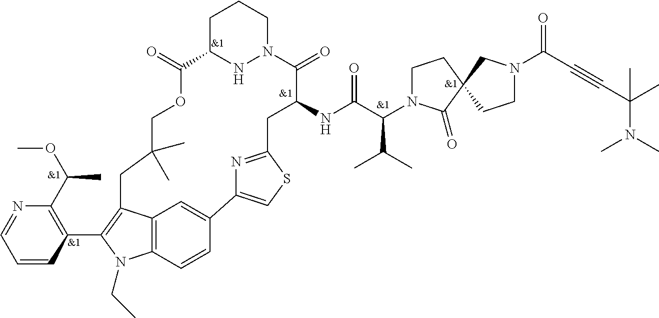 |
| B357 | 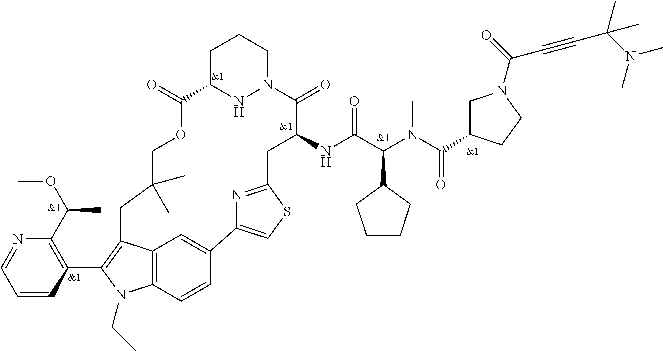 |
| B358 | 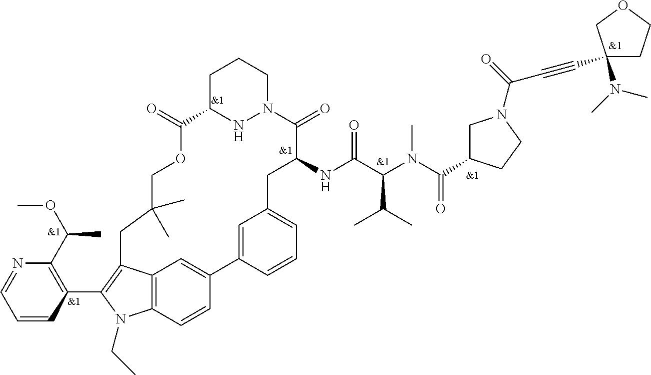 |
| B359 | 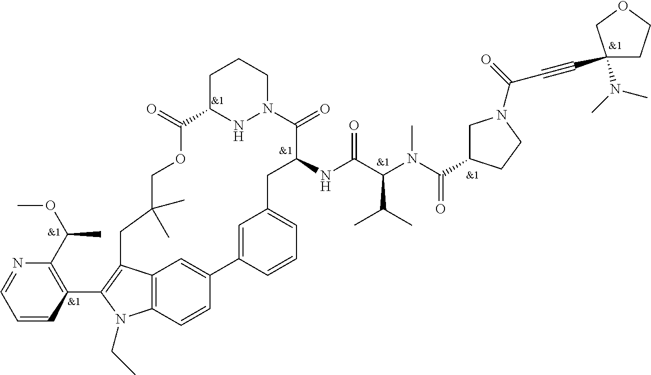 |
| B360 | 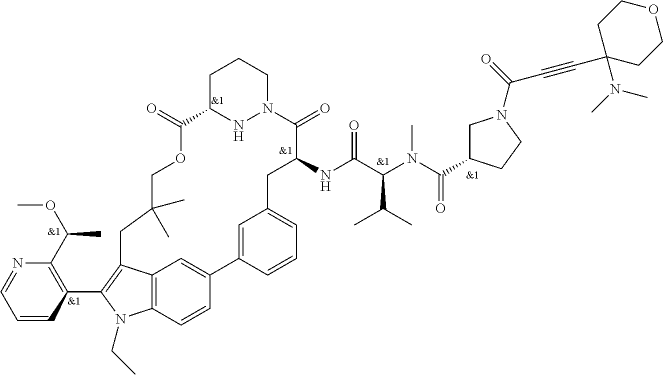 |
| B361 | 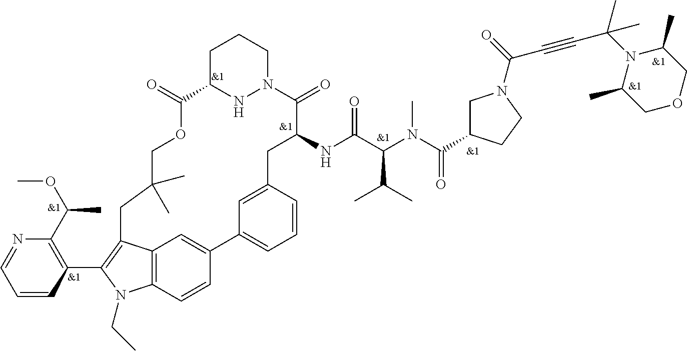 |
| B362 | 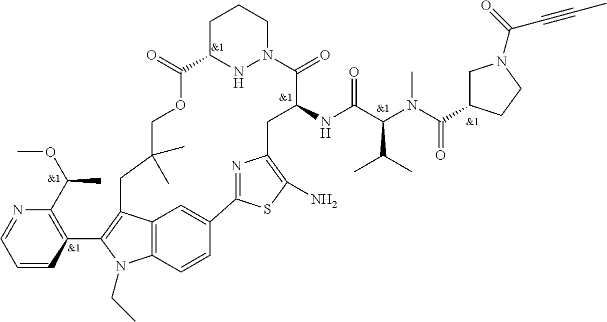 |
| B363 | 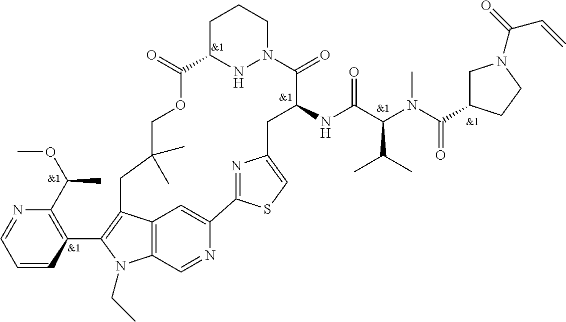 |
| B364 | 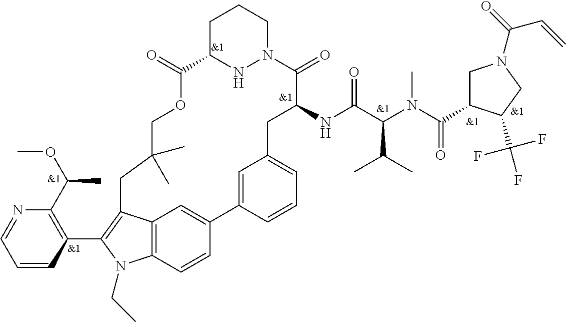 |
| B365 | 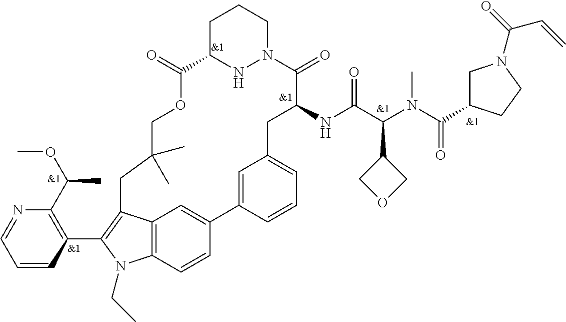 |
| B366 | 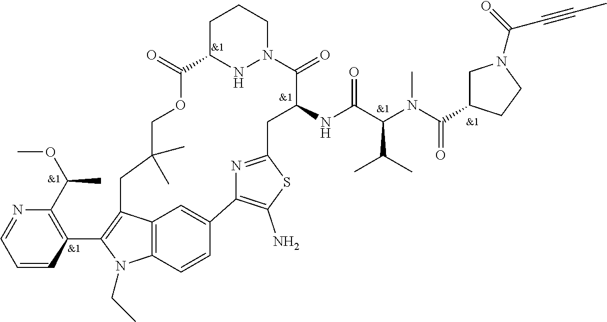 |
| B367 | 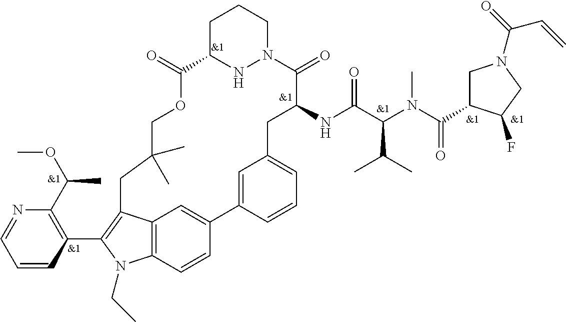 |
| B368 | 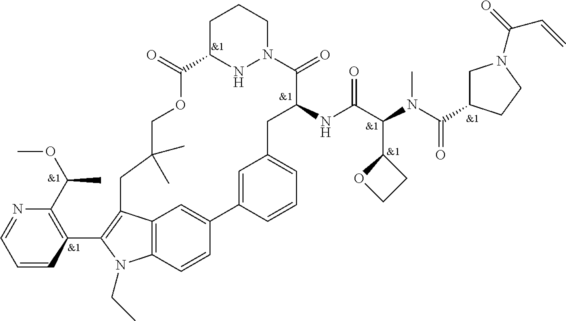 |
| B369 | 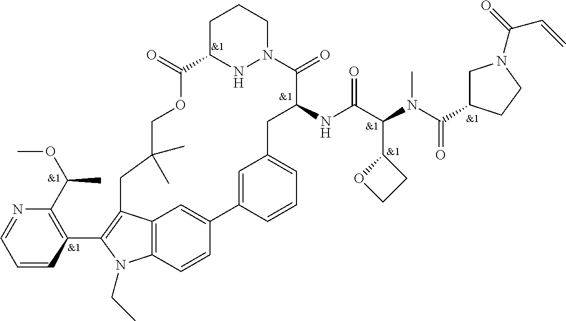 |
| B370 | 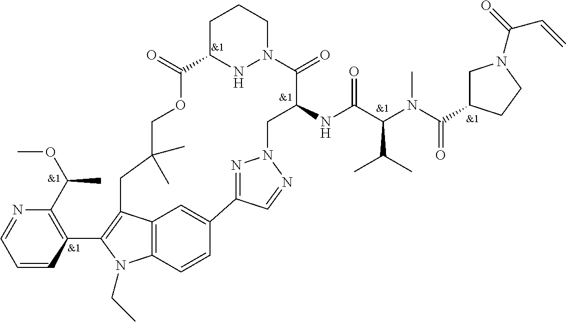 |
| B371 | 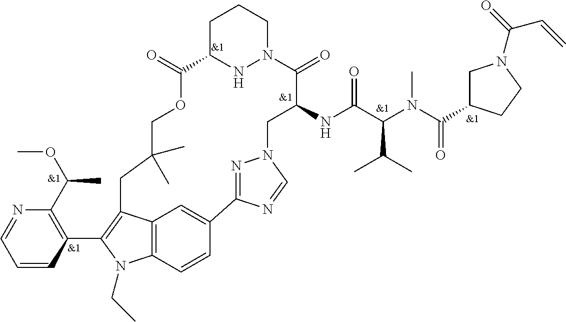 |
| B372 | 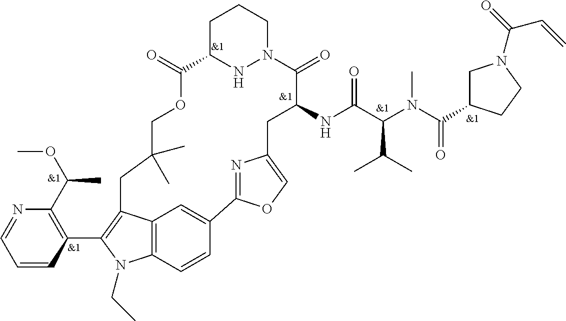 |
| B373 | 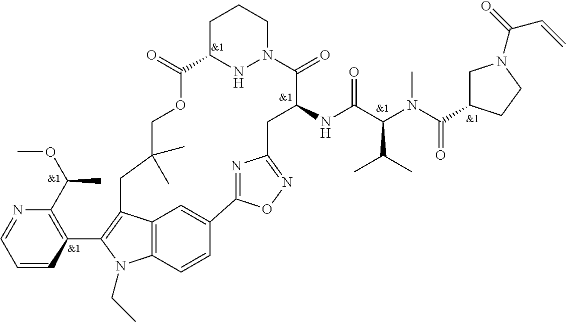 |
| B374 | 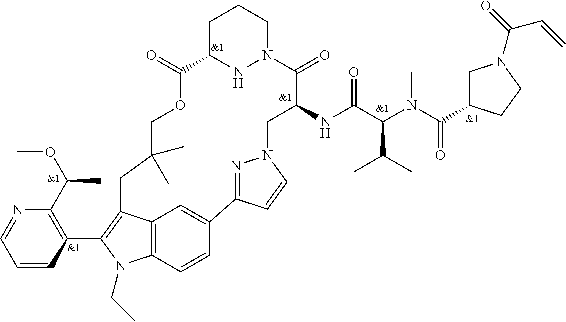 |
| B375 | 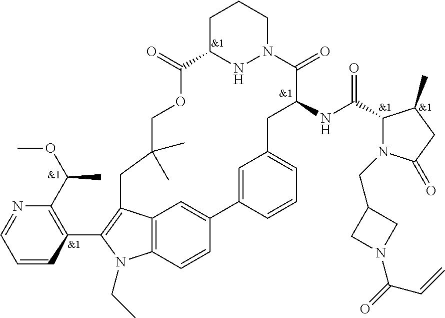 |
| B376 | 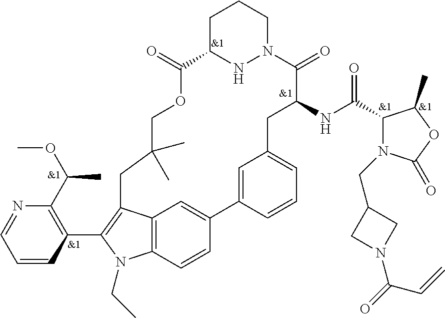 |
| B377 | 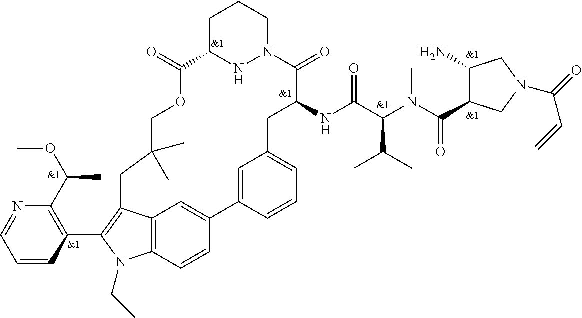 |
| B378 | 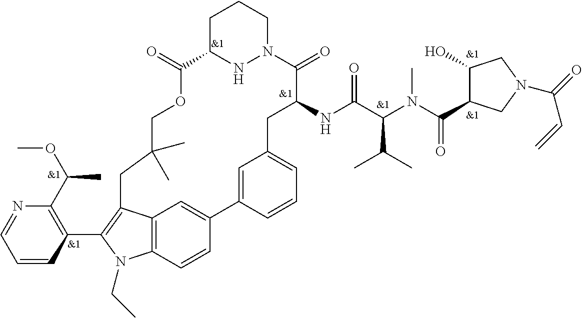 |
| B379 | 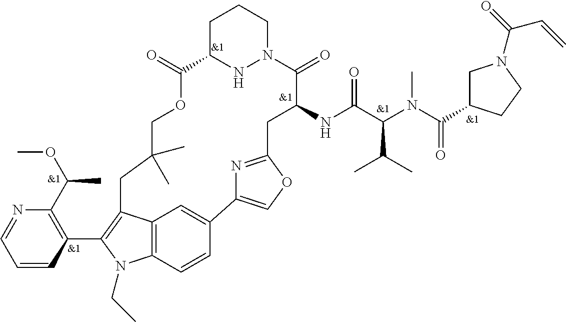 |
| B380 | 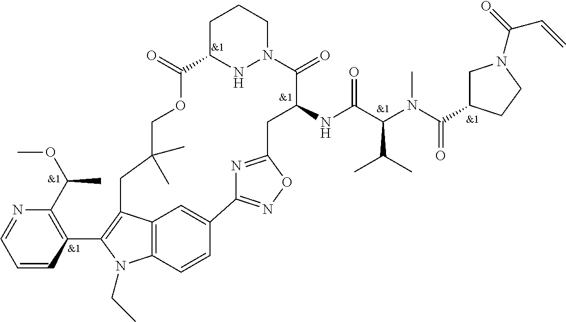 |
| B381 | 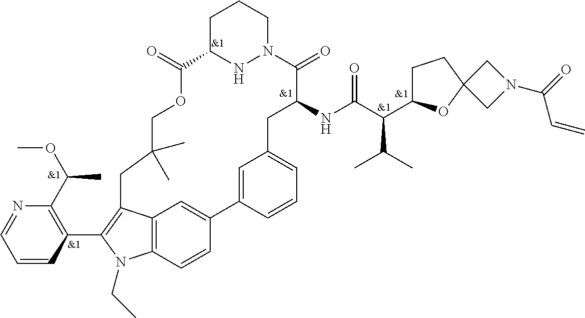 |
| B382 | 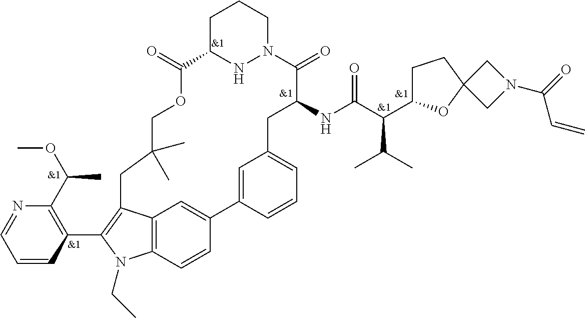 |
| B383 | 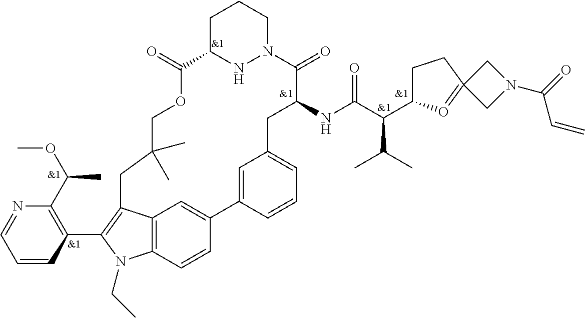 |
| B384 | 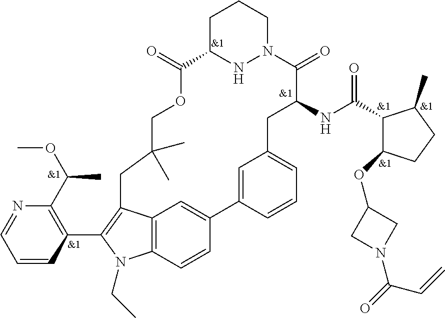 |
| B385 | 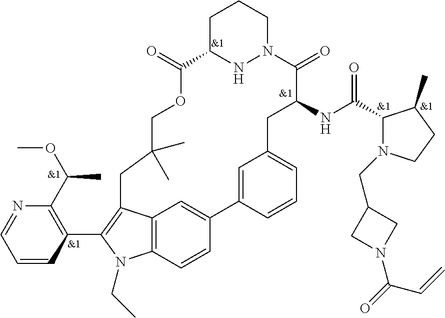 |
| B386 | 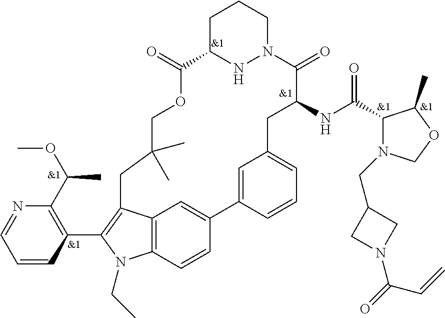 |
| B387 | 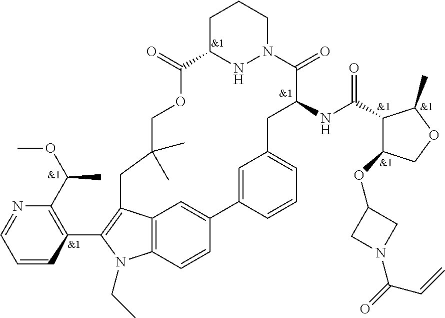 |
| B388 | 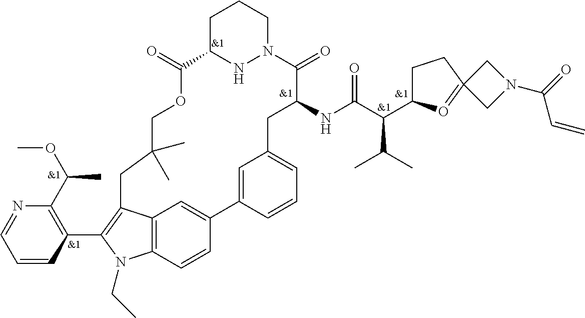 |
| B389 | 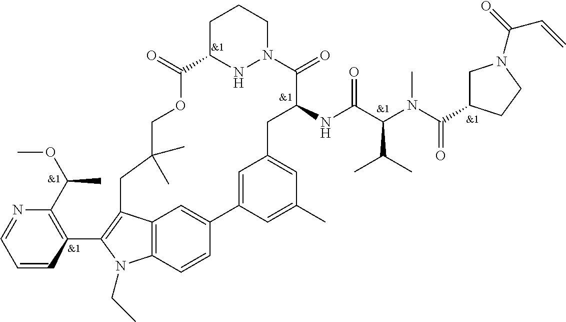 |
| B390 | 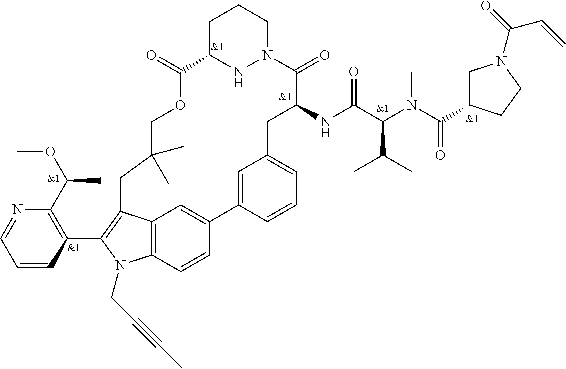 |
| B391 | 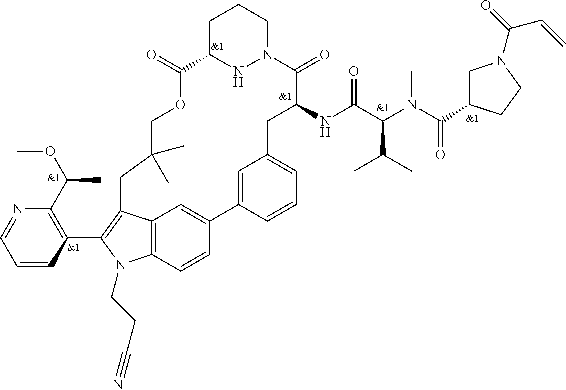 |
| B392 | 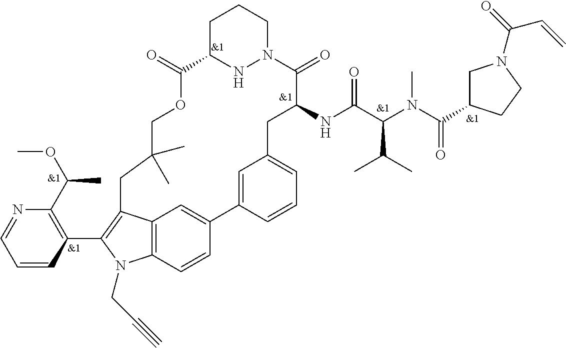 |
| B393 | 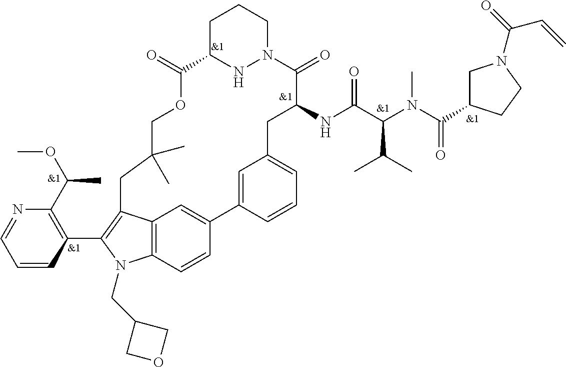 |
| B394 | 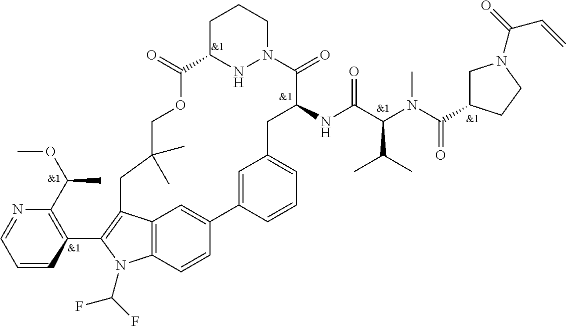 |
| B395 | 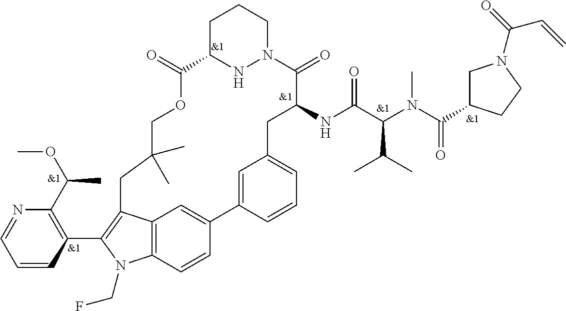 |
| B396 | 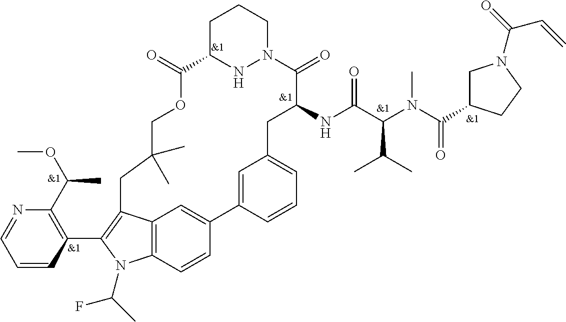 |
| B397 | 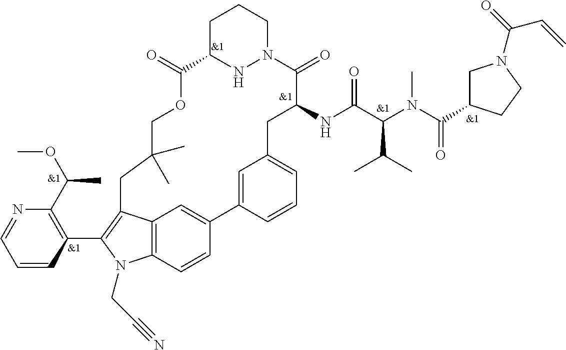 |
| B398 | 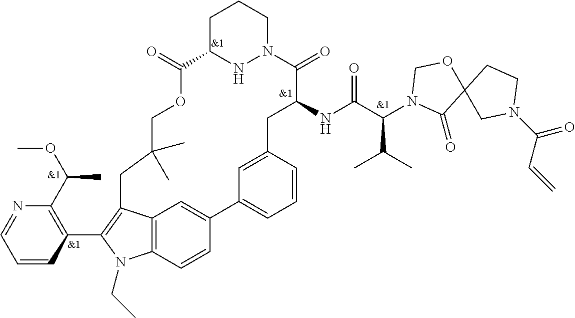 |
| B399 | 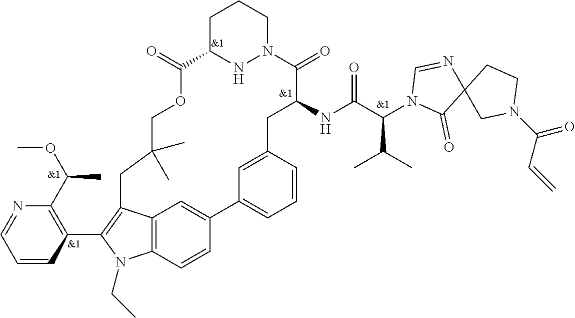 |
| B400 | 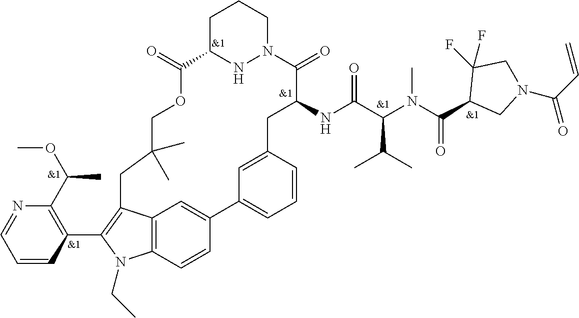 |
| B401 | 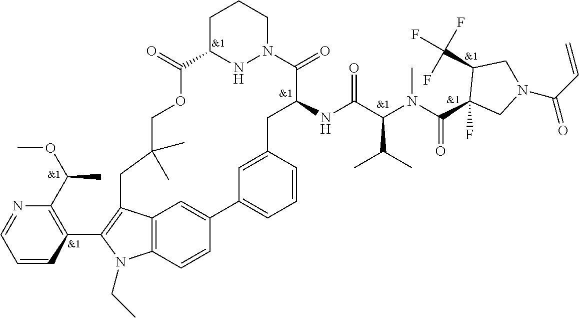 |
| B402 | 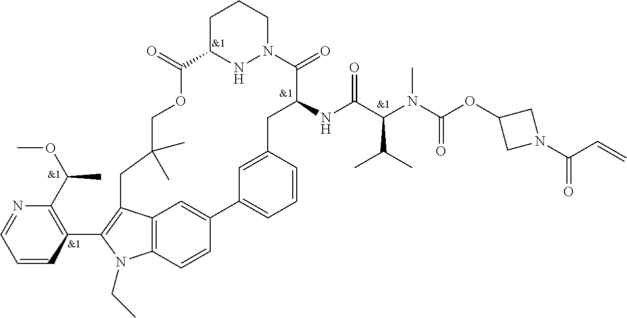 |
| B403 | 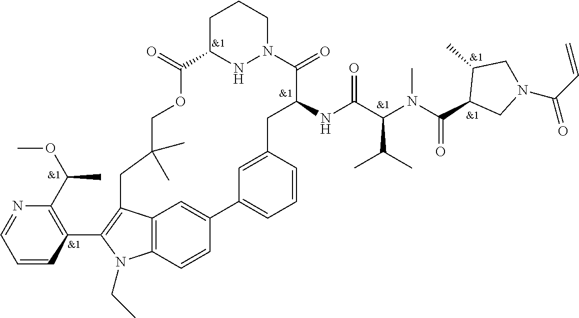 |
| B404 | 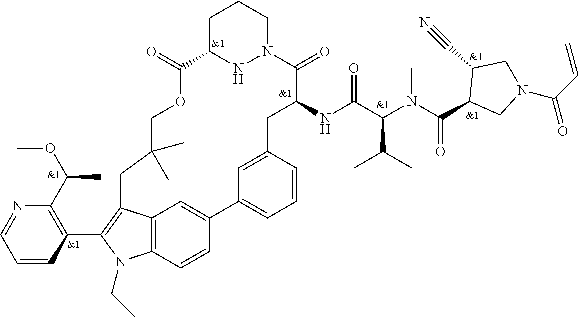 |
| B405 | 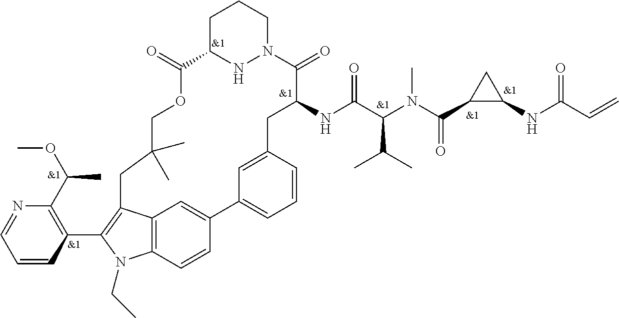 |
| B406 | 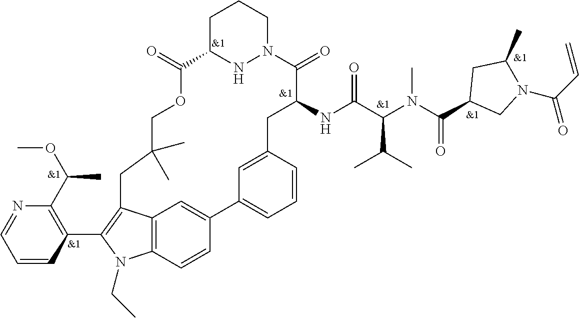 |
| B407 | 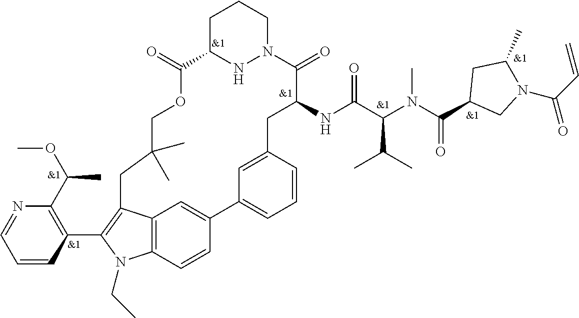 |
| B408 | 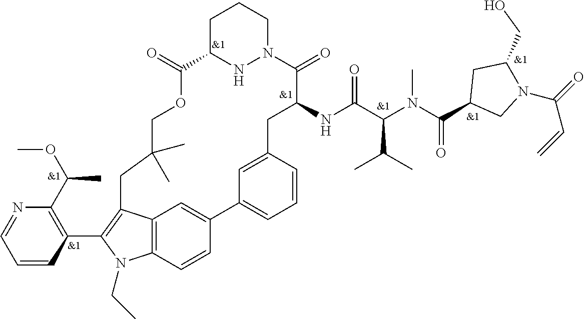 |
| B409 | 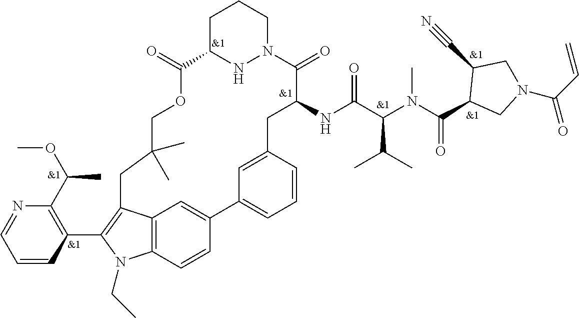 |
| B410 | 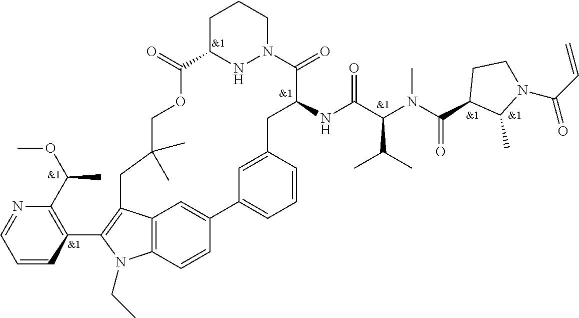 |
| B411 | 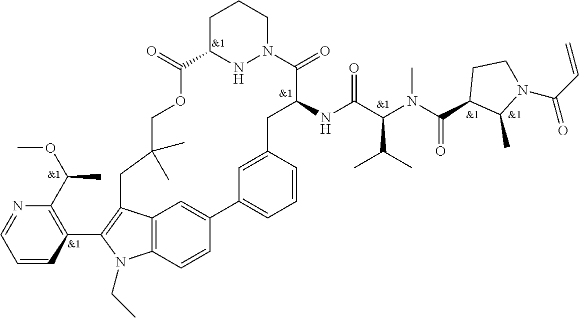 |
| B412 | 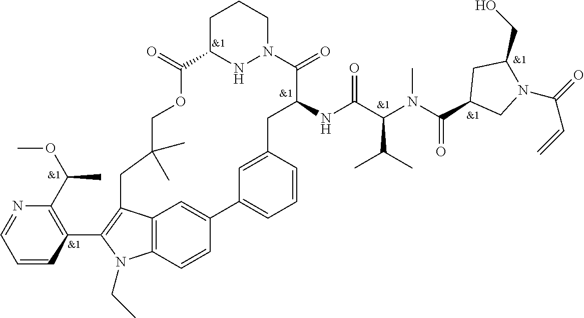 |
| B413 | 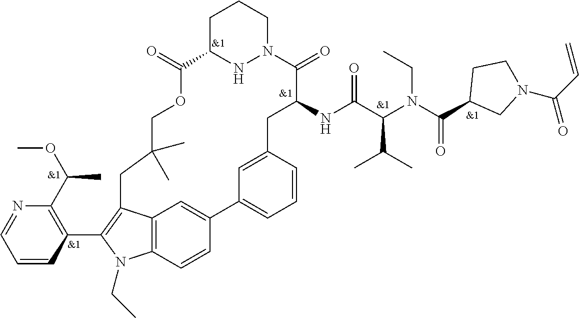 |
| B414 | 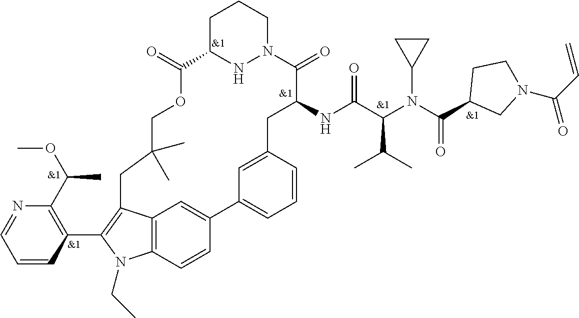 |
| B415 | 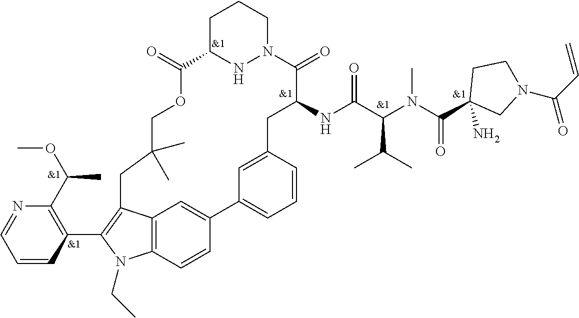 |
| B416 | 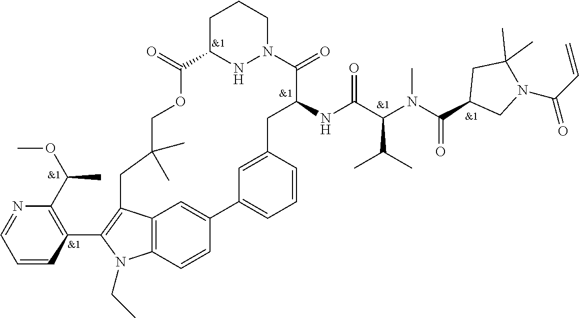 |
| B417 | 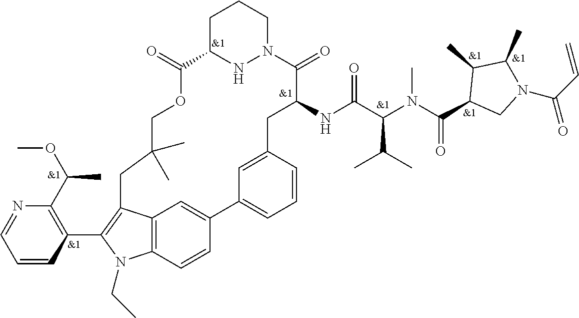 |
| B418 | 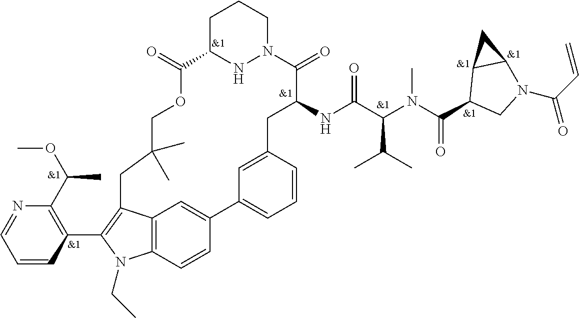 |
| B419 | 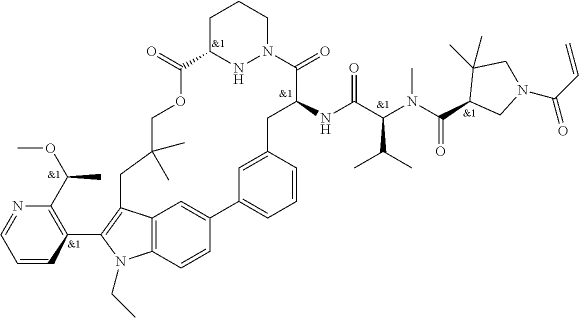 |
| B420 | 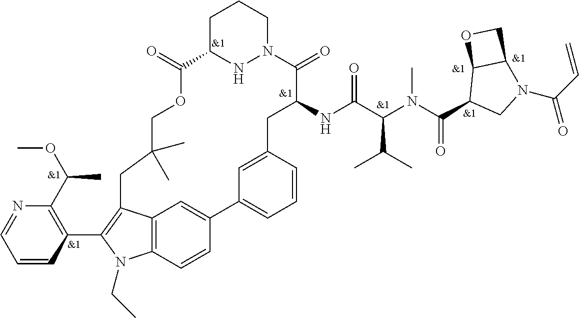 |
| B421 | 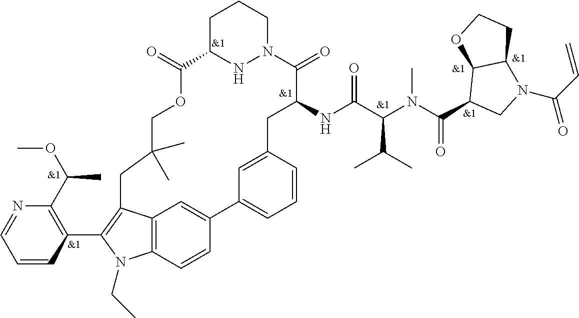 |
| B422 | 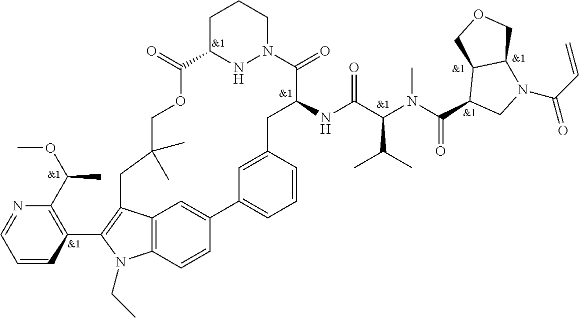 |
| B423 | 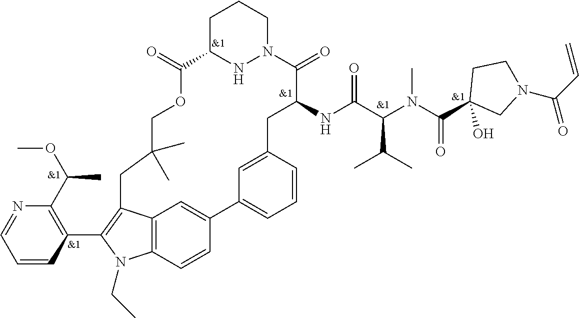 |
| B424 | 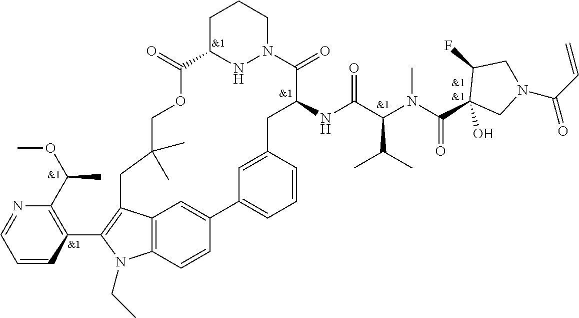 |
| B425 | 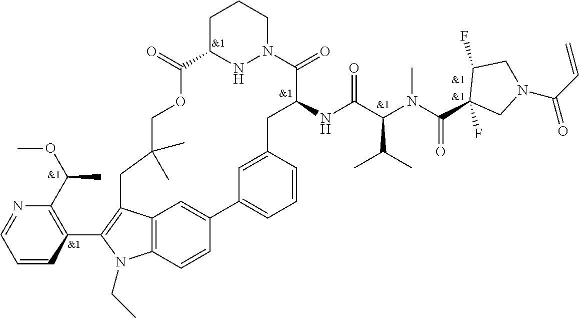 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Further provided is a conjugate, or salt thereof, comprising the structure of Formula IV:

M-L-P Formula IV

wherein L is a linker;

P is a monovalent organic moiety; and

M has the structure of Formula Va:

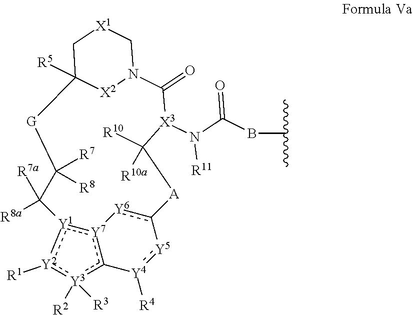

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH; n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R^{9′} is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo; and

R¹¹ is hydrogen or C₁-C₃ alkyl.

In some embodiments the conjugate, or salt thereof, comprises the structure of Formula IV:

M-L-P Formula IV

wherein L is a linker;

P is a monovalent organic moiety; and

M has the structure of Formula Vb:

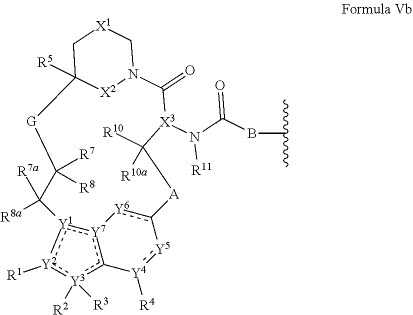

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo; and

R¹¹ is hydrogen or C₁-C₃ alkyl.

In some embodiments, the conjugate has the structure of Formula IV:

M-L-P Formula IV

wherein L is a linker;

P is a monovalent organic moiety; and

M has the structure of Formula Vc:

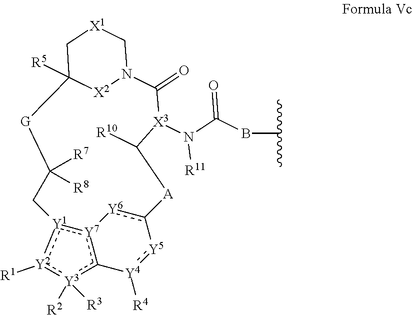

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y, and Y⁷ are, independently, C or N;

Y⁵ and Y are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments, a compound of the present invention has the structure of of Formula IV:

M-L-P Formula IV

wherein L is a linker;

P is a monovalent organic moiety; and

M has the structure of Formula Vd:

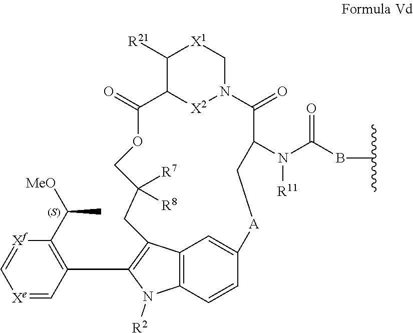

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

R² is C₁-C₆ alkyl, C₁-C₆ fluoroalkyl, or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} and X^{f} are, independently, N or CH:

R¹¹ is hydrogen or C₁-C₃ alkyl; and

R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of of Formula IV:

M-L-P Formula IV

wherein L is a linker;

P is a monovalent organic moiety; and

M has the structure of Formula Ve:

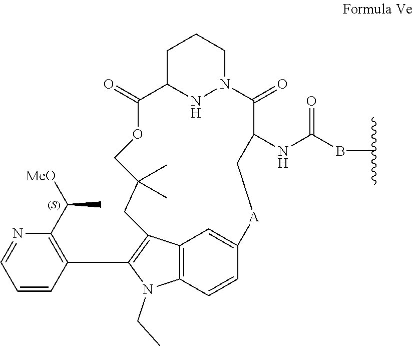

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a conjugate of the present invention, the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₃ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A². In some embodiments of a conjugate of the present invention, the monovalent organic moiety is a protein, such as a Ras protein. In some embodiments, the Ras protein is K-Ras G12C, K-Ras G13C, H-Ras G12C, H-Ras G13C, N-Ras G12C, or N-Ras G13C. Other Ras proteins are described herein. In some embodiments, the linker is bound to the monovalent organic moiety through a bond to a sulfhydryl group of an amino acid residue of the monovalent organic moiety. In some embodiments, the linker is bound to the monovalent organic moiety through a bond to a carboxyl group of an amino acid residue of the monovalent organic moiety.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as K-Ras G12C, K-Ras G13C, H-Ras G12C, H-Ras G13C, N-Ras G12C, or N-Ras G13C. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras G12C, K-Ras G13C, H-Ras G12C, H-Ras G13C, N-Ras G12C, or N-Ras G13C. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

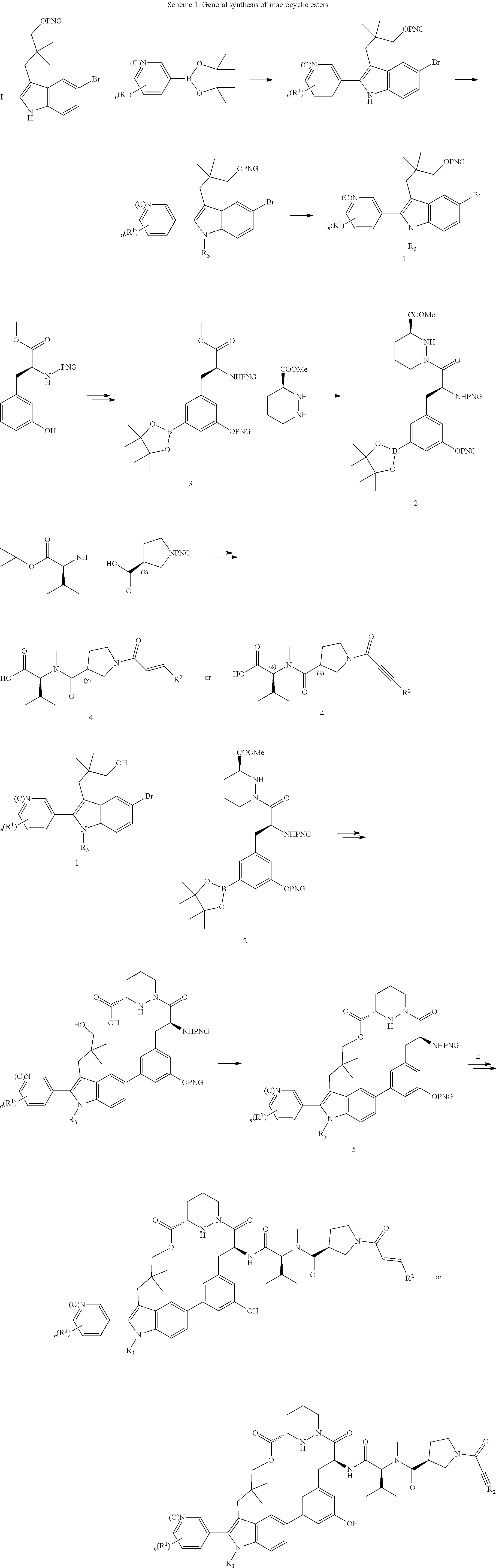

A general synthesis of macrocyclic esters is outlined in Scheme 1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions. Methyl-amino-hexahydropyridazine-3-carboxylate-boronic ester (2) can be prepared in three steps, including protection, iridium catalyst mediated borylation, and coupling with methyl methyl (S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

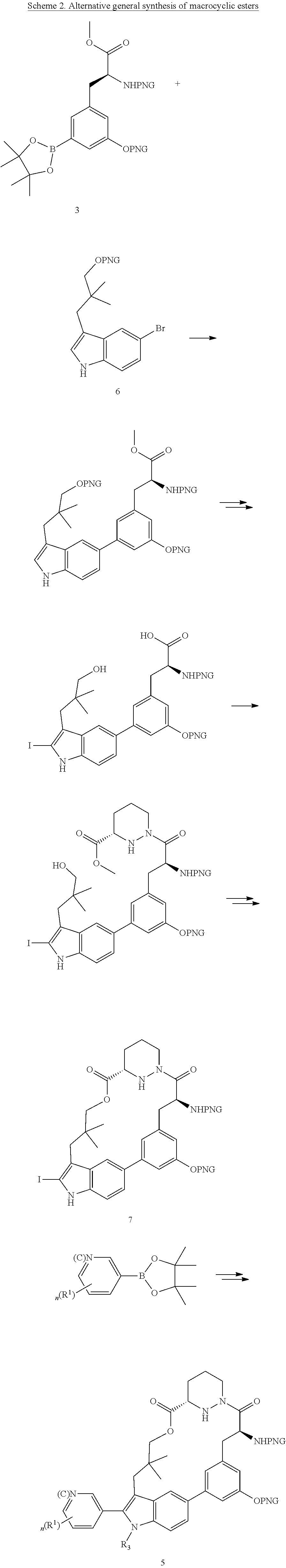

Alternatively, macrocyclic ester can be prepared as described in Scheme 2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkylation can yield fully protected macrocycle (5). Additional deprotection or functionalization steps are required to produce the final compound.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (I), where B, L and W are defined herein, including by using methods exemplified in the Example section herein.

Compounds of Table 1 herein were prepared using methods disclosed herein or were prepared using methods disclosed herein combined with the knowledge of one of skill in the art. Compounds of Table 2 may be prepared using methods disclosed herein or may be prepared using methods disclosed herein combined with the knowledge of one of skill in the art.

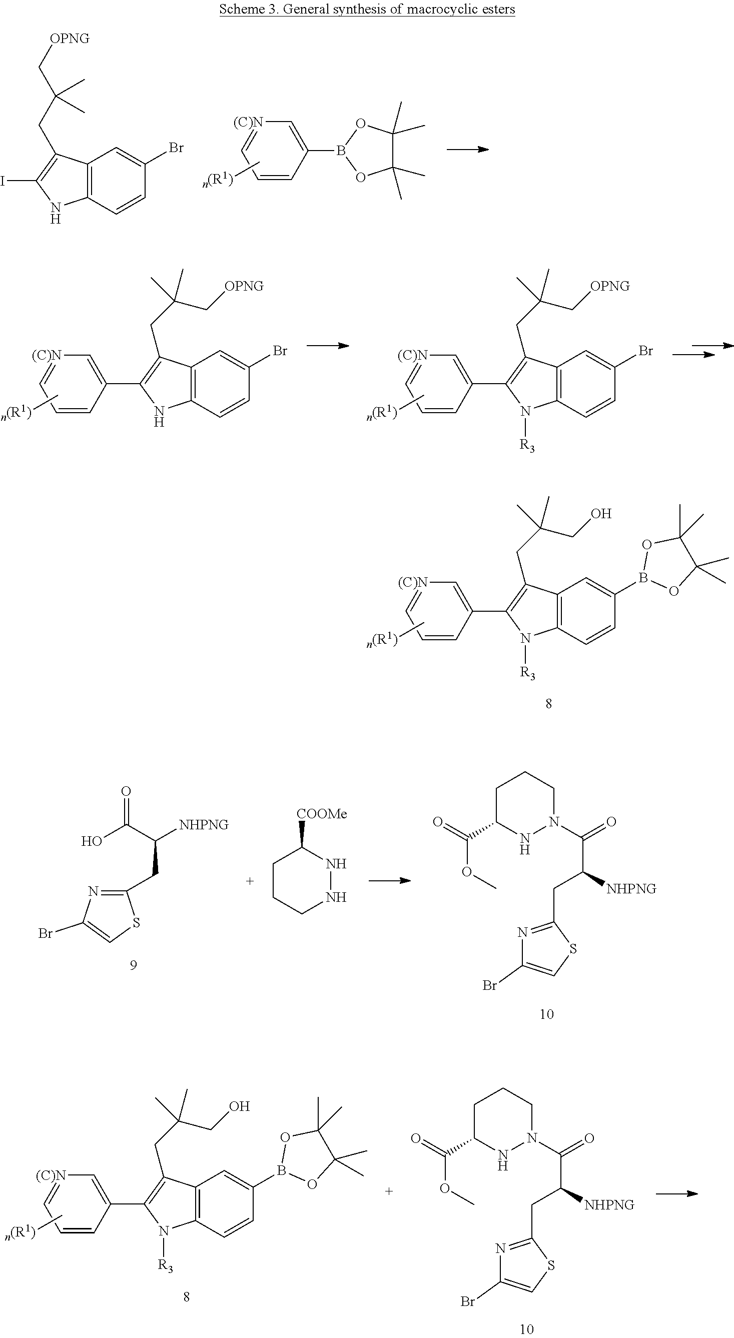

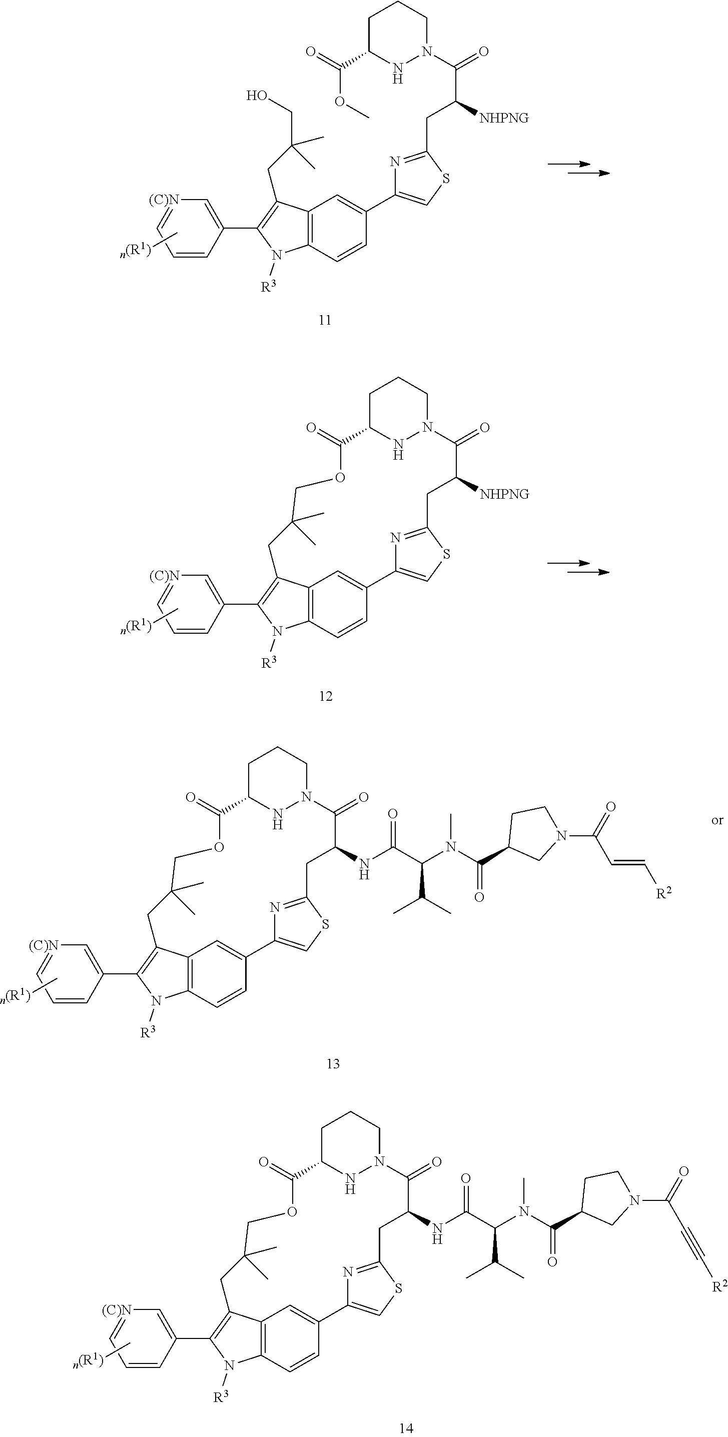

An alternative general synthesis of macrocyclic esters is outlined in Scheme 3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

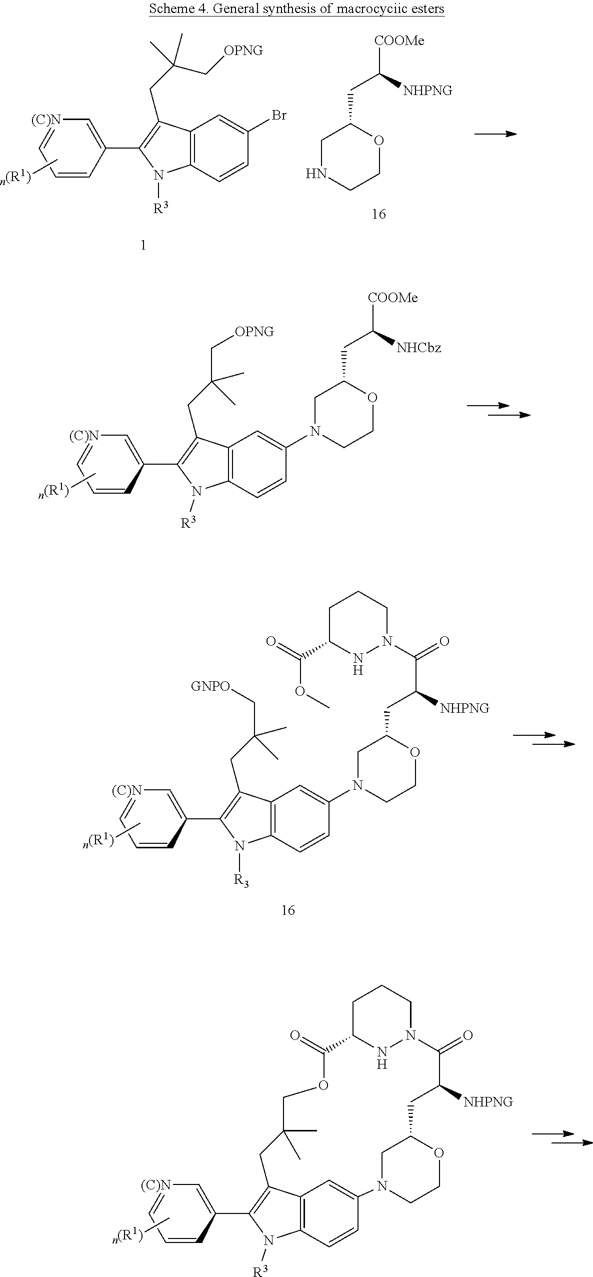

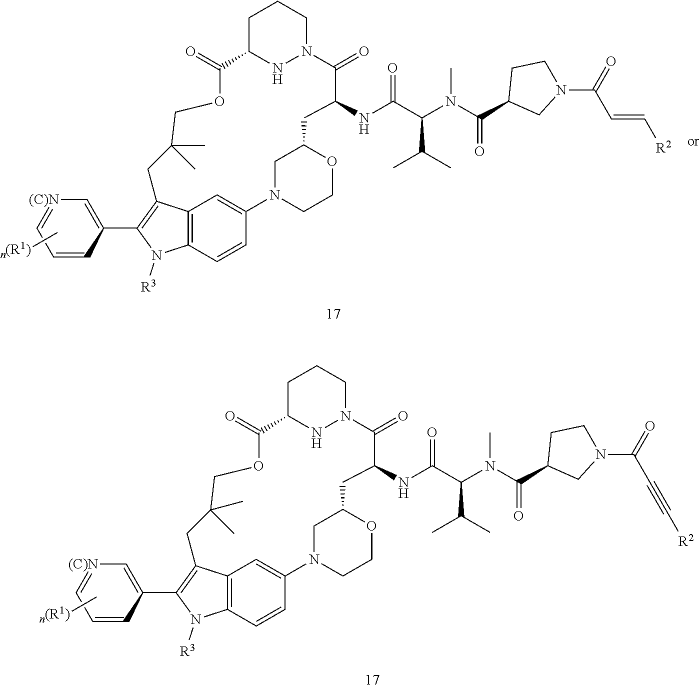

An alternative general synthesis of macrocyclic esters is outlined in Scheme 4. An appropriately substituted morpholine or an alternative herecyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

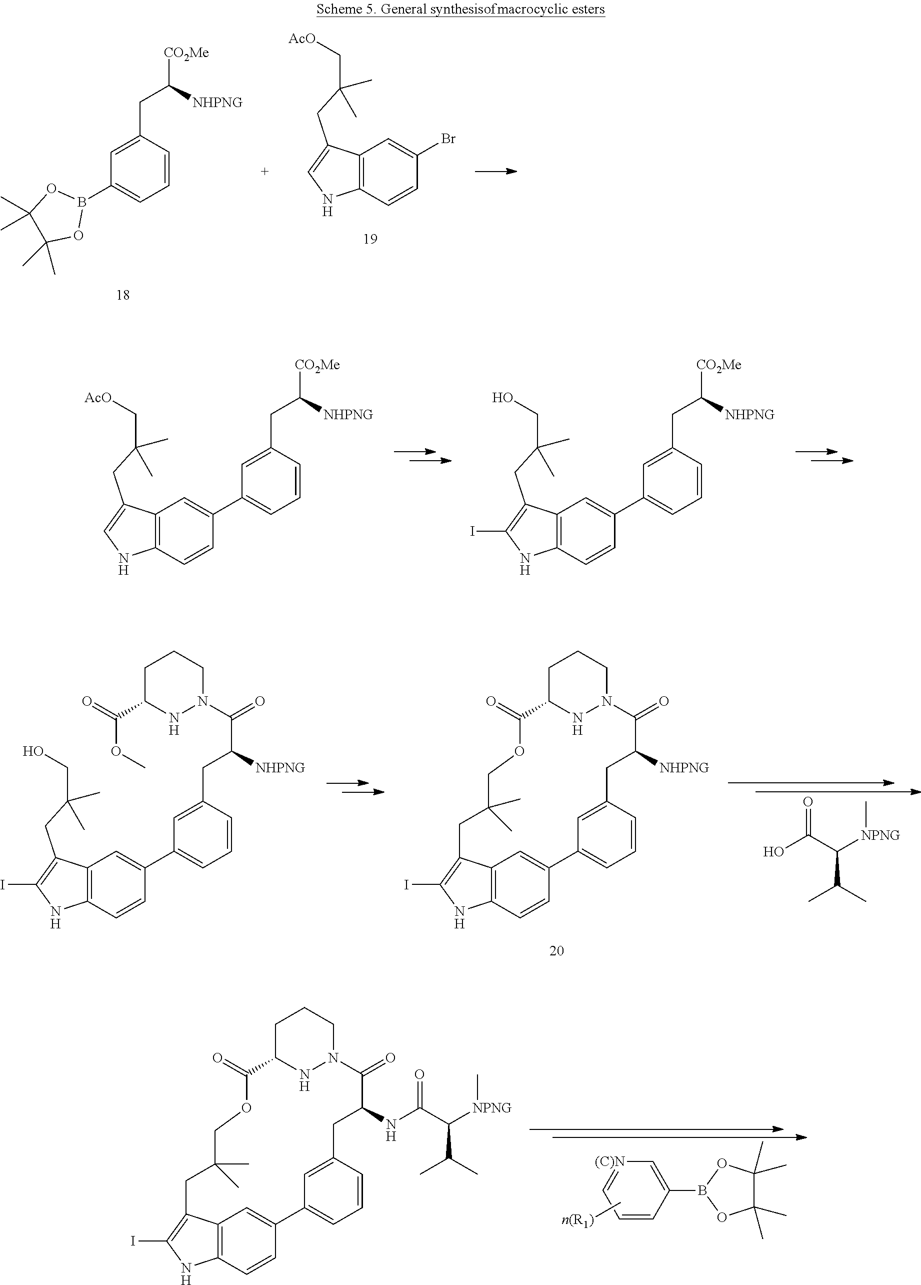

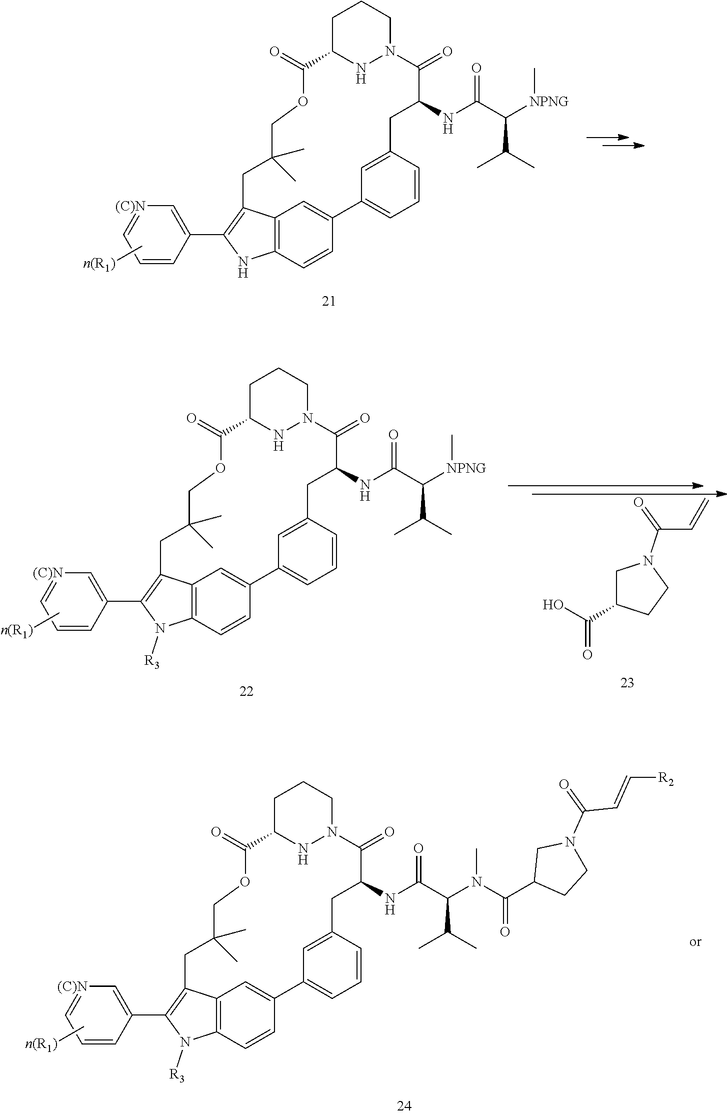

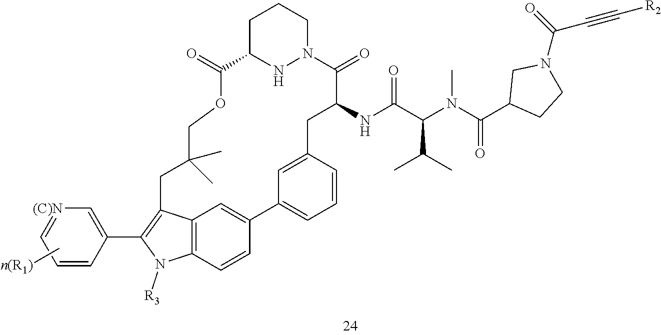

An alternative general synthesis of macrocyclic esters is outlined in Scheme 5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclizarion steps. Subsequent coupling with an appropriately substituted protected aminoacid followed by palladium mediated coupling yields intermediate 21. Additional deprotection and derivatization steps, including alkyllation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (I), where B, L and W are defined herein, including by using methods exemplified in the Example section herein.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Numbered Embodiments

[1] A compound, or pharmaceutically acceptable salt thereof, having the structure of Formula I:

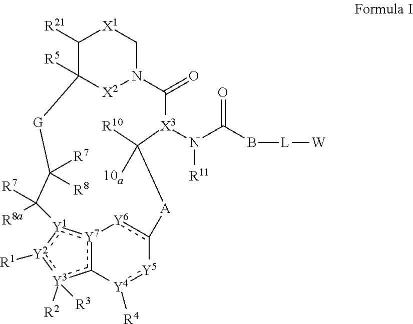

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R²¹ is H or C₁-C₃ alkyl.

[2] The compound, or pharmaceutically acceptable salt thereof, of paragraph [1], wherein G is optionally substituted C₁-C₄ heteroalkylene.

[3] The compound, or pharmaceutically acceptable salt thereof, of paragraph [1] or [2], wherein the compound has the structure of Formula Ic:

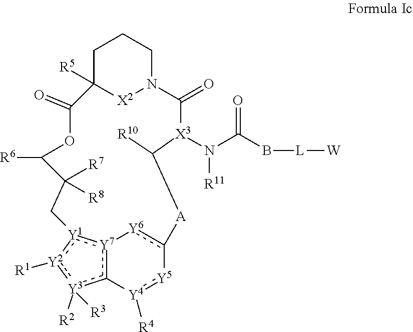

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

[4] The compound, or pharmaceutically acceptable salt thereof, of any one of paragraphs [1] to [3], wherein X² is NH.

[5] The compound, or pharmaceutically acceptable salt thereof, of any one of paragraphs [1] to [4], wherein X³ is CH.

[6] The compound, or pharmaceutically acceptable salt thereof, of any one of paragraphs [1] to [5], wherein R¹¹ is hydrogen.

[7] The compound, or pharmaceutically acceptable salt thereof, of any one of paragraphs [1] to [5], wherein R¹¹ is C₁-C₃ alkyl.

[8] The compound, or pharmaceutically acceptable salt thereof, of paragraph [7], wherein R¹¹ is methyl.

[9] The compound, or pharmaceutically acceptable salt thereof, of any one of paragraphs [1] to [6], wherein the compound has the structure of Formula Id:

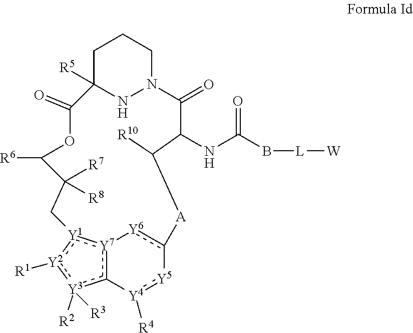

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

[10] The compound, or pharmaceutically acceptable salt thereof, of any one of paragraphs [1] to [9] wherein X¹ is optionally substituted C₁-C₂ alkylene.

[11] The compound, or pharmaceutically acceptable salt thereof, of paragraph [10], wherein X¹ is methylene.

[12] The compound, or pharmaceutically acceptable salt thereof, of any one of paragraphs [1] to [11], wherein R⁵ is hydrogen.

[13] The compound, or pharmaceutically acceptable salt thereof, of any one of paragraphs [1] to [11], wherein R⁵ is C₁-C₄ alkyl optionally substituted with halogen.

[14] The compound, or pharmaceutically acceptable salt thereof, of paragraph [13], wherein R⁵ is methyl.

[15] The compound, or pharmaceutically acceptable salt thereof, of any one of paragraphs [1] to [14], wherein Y⁴ is C.

[16] The compound, or pharmaceutically acceptable salt thereof, of any one of paragraphs [1] to [15], wherein R⁴ is hydrogen.

[17] The compound, or pharmaceutically acceptable salt thereof, of any one of paragraphs [1] to [16], wherein Y⁵ is CH.

[18] The compound, or pharmaceutically acceptable salt thereof, of any one of paragraphs [1] to [17], wherein Ye is CH.

[19] The compound, or pharmaceutically acceptable salt thereof, of any one of paragraphs [1] to [18], wherein Y¹ is C.

[20] The compound, or pharmaceutically acceptable salt thereof, of any one of paragraphs [1] to [19] wherein Y² is C.

[21] The compound, or pharmaceutically acceptable salt thereof, of any one of paragraphs [1] to [20] wherein Y³ is N.

[22] The compound, or pharmaceutically acceptable salt thereof, of any one of paragraphs [1] to [21], wherein R³ is absent.

[23] The compound, or pharmaceutically acceptable salt thereof, of any one of paragraphs [1] to [22], wherein Y⁷ is C.

[24] The compound, or pharmaceutically acceptable salt thereof, of any one of paragraphs [1] to [6] or [9] to [23], wherein the compound has the structure of Formula Ie:

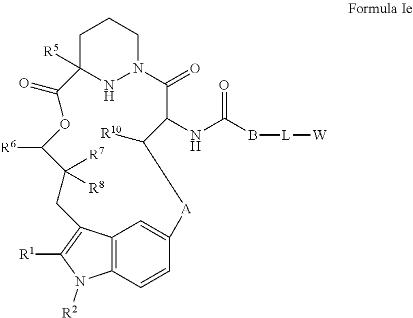

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

[25] The compound, or pharmaceutically acceptable salt thereof, of any one of paragraphs [3] to [24], wherein R⁸ is hydrogen.

[26] The compound, or pharmaceutically acceptable salt thereof, of any one of paragraphs [1] to [25], wherein R² is hydrogen, cyano, optionally substituted C₁-C₆ alkyl, optionally substituted 3 to 6-membered cycloalkyl, or optionally substituted 3 to 6-membered heterocycloalkyl.

[27] The compound, or pharmaceutically acceptable salt thereof, of paragraph [26], wherein R² is optionally substituted C₁-C₆ alkyl.

[28] The compound, or pharmaceutically acceptable salt thereof, of paragraph [27], wherein R² is ethyl.

[29] The compound, or pharmaceutically acceptable salt thereof, of any one of paragraphs [1] to [28], wherein R⁷ is optionally substituted C₁-C₃ alkyl.

[30] The compound, or pharmaceutically acceptable salt thereof, of paragraph 29, wherein R⁷ is C₁-C₃ alkyl.

[31] The compound, or pharmaceutically acceptable salt thereof, of any one of paragraphs [1] to 30, wherein R⁸ is optionally substituted C₁-C₃ alkyl.

[32] The compound, or pharmaceutically acceptable salt thereof, of paragraph [31], wherein R⁸ is C₁-C₃ alkyl.

[33] The compound, or pharmaceutically acceptable salt thereof, of any one of paragraphs [1] to [32], wherein the compound has the structure of Formula If:

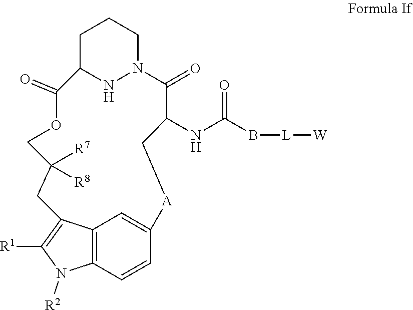

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

[34] The compound, or pharmaceutically acceptable salt thereof, of any one of paragraphs [1] to [33], wherein R¹ is optionally substituted 6 to 10-membered aryl, optionally substituted 3 to 6-membered cycloalkenyl, or optionally substituted 5 to 10-membered heteroaryl.

[35] The compound, or pharmaceutically acceptable salt thereof, of paragraph [34], wherein R¹ is optionally substituted 6-membered aryl, optionally substituted 6-membered cycloalkenyl, or optionally substituted 6-membered heteroaryl.

[36] The compound, or pharmaceutically acceptable salt thereof, of paragraph [35], wherein R¹ is

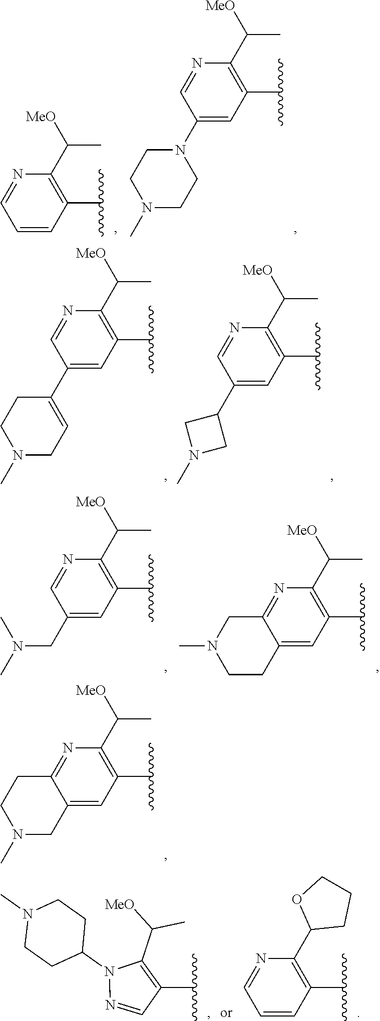

[37] The compound, or pharmaceutically acceptable salt thereof, of paragraph [36], wherein R¹ is

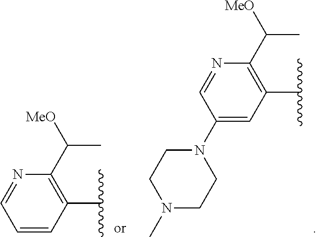 [SMILES: *c1cccnc1C(C)OC ; *c1cc(N2CCN(C)CC2)cnc1C(C)OC]

[38] The compound, or pharmaceutically acceptable salt thereof, of any one of paragraphs [1] to [37], wherein the compound has the structure of Formula Ig:

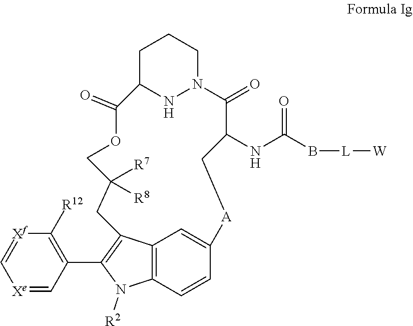

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

R² is C₁-C₆ alkyl, C₁-C₆ fluoroalkyl, or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl

X^{e} and X^{f} are, independently, N or CH; and

R¹² is optionally substituted C₁-C₆ alkyl, optionally substituted C₁-C₆ heteroalkyl, or optionally substituted 3 to 7-membered heterocycloalkyl.

[39] The compound, or pharmaceutically acceptable salt thereof, of paragraph [38], wherein X^{e} is N and X^{f} is CH.

[40] The compound, or pharmaceutically acceptable salt thereof, of paragraph [38], wherein X^{e} is CH and X^{f} is N.

[41] The compound, or pharmaceutically acceptable salt thereof, of any one of paragraphs [38] to [40], wherein R¹² is optionally substituted C₁-C₆ heteroalkyl.

[42] The compound, or pharmaceutically acceptable salt thereof, of any one of paragraphs [38] to [41], wherein R¹² is

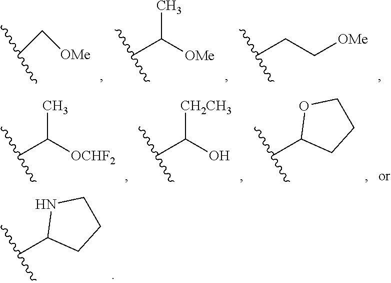

[43] The compound, or pharmaceutically acceptable salt thereof, of paragraph [1] or [2], wherein the compound has the structure of Formula VI:

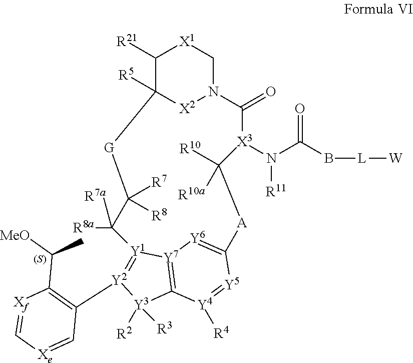

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₃ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is a cross-linking group comprising a vinyl ketone, a vinyl sulfone, an ynone, a haloacetal, or an alkynyl sulfone;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl;

R²¹ is hydrogen or C₁-C₃ alkyl (e.g., methyl); and

X^{e} and X^{f} are, independently, N or CH.

[44] The compound, or pharmaceutically acceptable salt thereof, of paragraph [43], wherein the compound has the structure of Formula VIa:

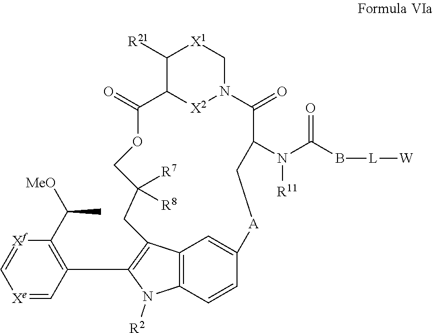

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is a cross-linking group comprising a vinyl ketone, a vinyl sulfone, an ynone, or an alkynyl sulfone;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

R² is C₁-C₆ alkyl, C₁-C₆ fluoroalkyl, or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} and X^{f} are, independently, N or CH;

R¹¹ is hydrogen or C₁-C₃ alkyl; and

R²¹ is hydrogen or C₁-C₃ alkyl.

[45] The compound, or pharmaceutically acceptable salt thereof, of paragraph [43] or [44], wherein the compound has the structure of Formula VIb:

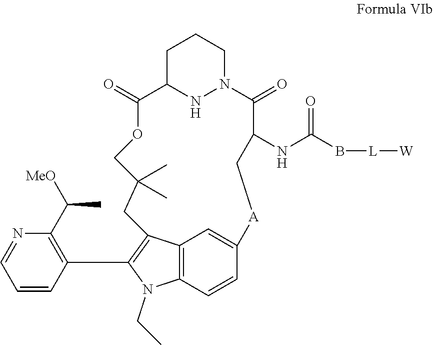

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

L is absent or a linker; and

W is a cross-linking group comprising a vinyl ketone, a vinyl sulfone, an ynone, or an alkynyl sulfone.

[46] The compound, or pharmaceutically acceptable salt thereof, of any one of paragraphs [1] to [45], wherein A is optionally substituted 6-membered arylene.

[47] The compound, or pharmaceutically acceptable salt thereof, of paragraph [46], wherein A has the structure:

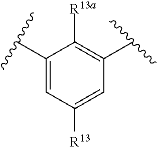

wherein R¹³ is hydrogen, halo, hydroxy, amino, optionally substituted C₁-C₆ alkyl, or optionally substituted C₁-C₆ heteroalkyl; and

R^{13a} is hydrogen or halo.

[48] The compound, or pharmaceutically acceptable salt thereof, of paragraph [47], wherein R¹³ and R^{13a} are each hydrogen.

[49] The compound, or pharmaceutically acceptable salt thereof, of paragraph [47], wherein R¹³ is hydroxy, methyl, fluoro, or difluoromethyl.

[50] The compound, or pharmaceutically acceptable salt thereof, of any one of paragraphs [1] to [45], wherein A is optionally substituted 5 to 6-membered heteroarylene.

[51] The compound, or pharmaceutically acceptable salt thereof, of paragraph [50], wherein A is:

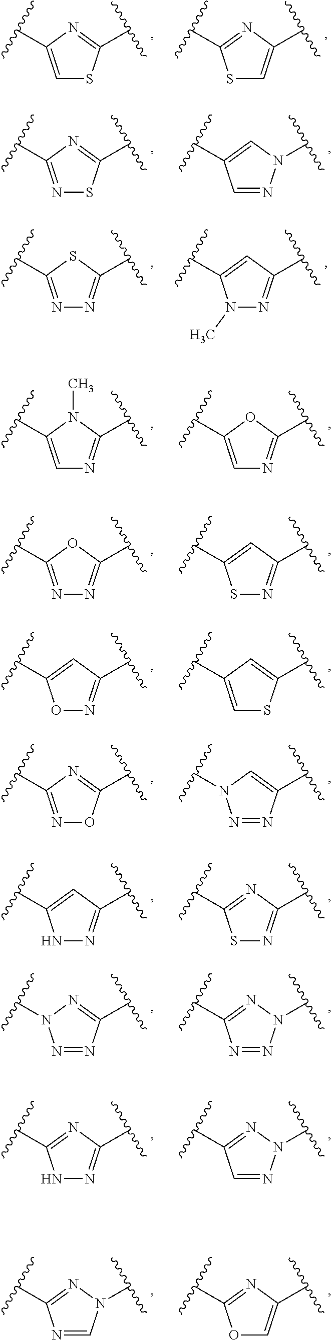

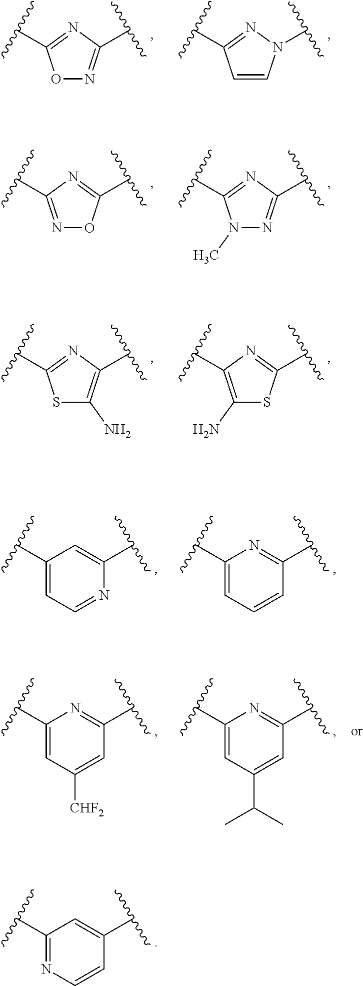

[52] The compound, or pharmaceutically acceptable salt thereof, of any one of paragraphs [1] to [45], wherein A is optionally substituted C₁-C₄ heteroalkylene.

[53] The compound, or pharmaceutically acceptable salt thereof, of paragraph [52], wherein A is:

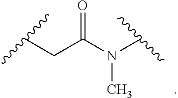 [SMILES: *CC(=O)N(*)C]

[54] The compound, or pharmaceutically acceptable salt thereof, of any one of paragraphs [1] to [45], wherein A is optionally substituted 3 to 6-membered heterocycloalkylene.

[55] The compound, or pharmaceutically acceptable salt thereof, of paragraph [54], wherein A is:

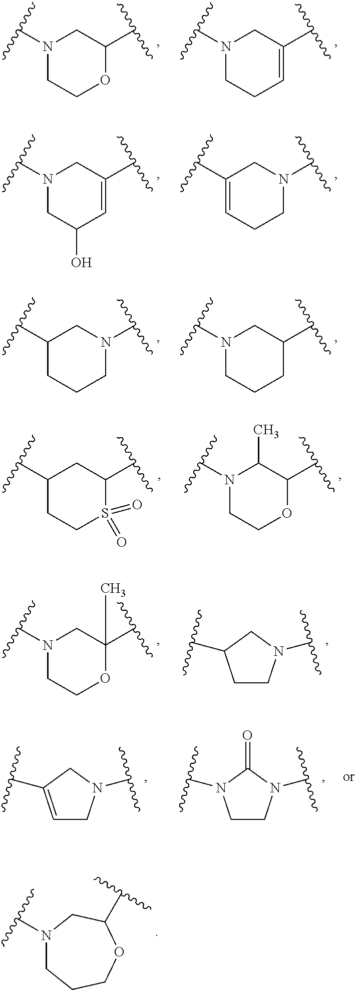

[56] The compound, or pharmaceutically acceptable salt thereof, of paragraph [55], wherein A is

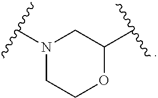 [SMILES: *C1CN(*)CCO1]

[57] The compound, or pharmaceutically acceptable salt thereof, of any one of paragraphs [1] to [56], wherein B is —CHR⁹—.

[58] The compound, or pharmaceutically acceptable salt thereof, of paragraph [57], wherein R⁹ is F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

[59] The compound, or pharmaceutically acceptable salt thereof, of paragraph [58], wherein R⁹ is:

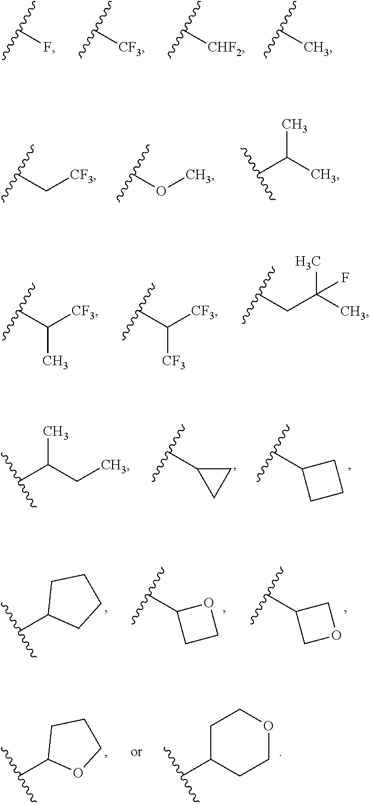

[60] The compound, or pharmaceutically acceptable salt thereof, of paragraph [59], wherein R⁹ is

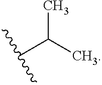 [SMILES: *C(C)C]

[61] The compound, or pharmaceutically acceptable salt thereof, of any one of paragraphs [1] to [56], wherein B is optionally substituted 6-membered arylene.

[62] The compound, or pharmaceutically acceptable salt thereof, of paragraph [61], wherein B is 6-membered arylene.

[63] The compound, or pharmaceutically acceptable salt thereof, of paragraph [61], wherein B is:

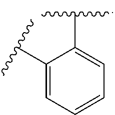 [SMILES: *c1ccccc1*]

[64] The compound, or pharmaceutically acceptable salt thereof, of any one of paragraphs [1] to [63], wherein R⁷ is methyl.

[65] The compound, or pharmaceutically acceptable salt thereof, of any one of paragraphs [1] to [64], wherein R⁸ is methyl.

[66] The compound, or pharmaceutically acceptable salt thereof, of any one of paragraphs [1] to [65], wherein the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B2, B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A².

[67] The compound, or a pharmaceutically acceptable salt thereof, of any one of paragraphs [1] to [66], wherein the linker is acyclic.

[68] The compound, or a pharmaceutically acceptable salt thereof, of paragraph [67], wherein the linker has the structure of Formula IIa:

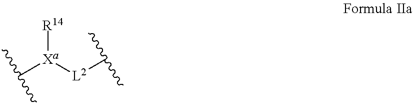

wherein X^{a} is absent or N;

R¹⁴ is absent, hydrogen or optionally substituted C₁-C₆ alkyl; and

L² is absent, —SO₂—, optionally substituted C₁-C₄ alkylene or optionally substituted C₁-C₄ heteroalkylene,

wherein at least one of X^{a}, R¹⁴, or L² is present.

[69] The compound, or a pharmaceutically acceptable salt thereof, of paragraph [68], wherein the linker has the structure:

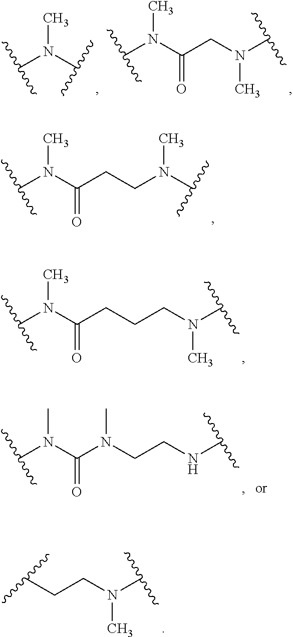

[70] The compound, or a pharmaceutically acceptable salt thereof, of any one of paragraphs [1] to [66], wherein the linker is or comprises a cyclic moiety.

[71] The compound, or a pharmaceutically acceptable salt thereof, of paragraph [70], wherein the linker has the structure of Formula IIb:

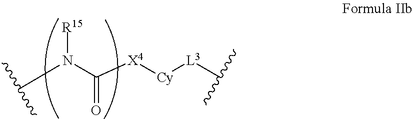

wherein o is 0 or 1;

R¹⁵ is hydrogen, optionally substituted C₁-C₆ alkyl, optionally substituted 3 to 8-membered cycloalkylene, or optionally substituted 3 to 8-membered heterocycloalkylene;

X⁴ is absent, optionally substituted C₁-C₄ alkylene, O, NCH₃, or optionally substituted C₁-C₄ heteroalkylene;

Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —SO₂—, optionally substituted C₁-C₄ alkylene or optionally substituted C₁-C₄ heteroalkylene.

[72] The compound, or a pharmaceutically acceptable salt thereof, of paragraph [71], wherein the linker has the structure:

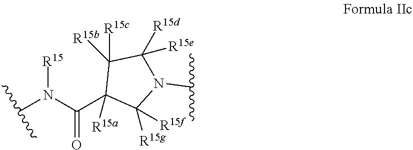

wherein R¹⁵ is hydrogen, optionally substituted C₁-C₆ alkyl, optionally substituted 3 to 8-membered cycloalkylene, or optionally substituted 3 to 8-membered heterocycloalkylene; and

R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

[73] The compound, or a pharmaceutically acceptable salt thereof, of paragraph [72], wherein the linker has the structure:

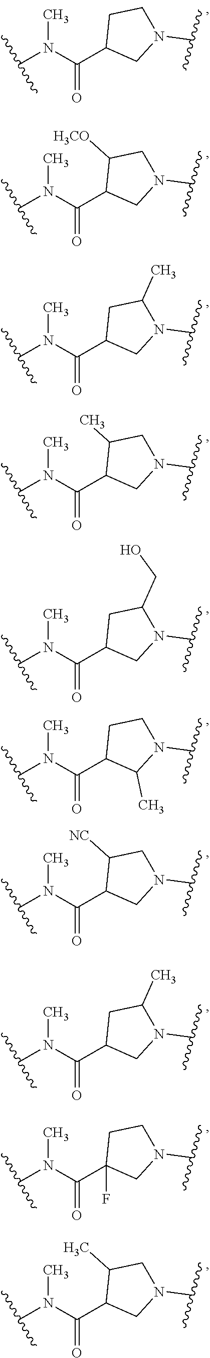

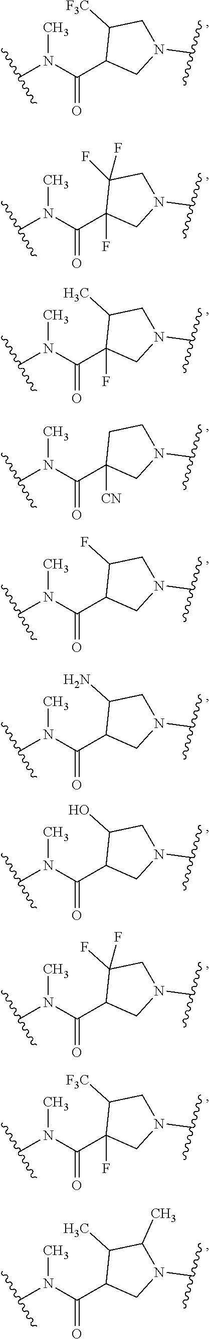

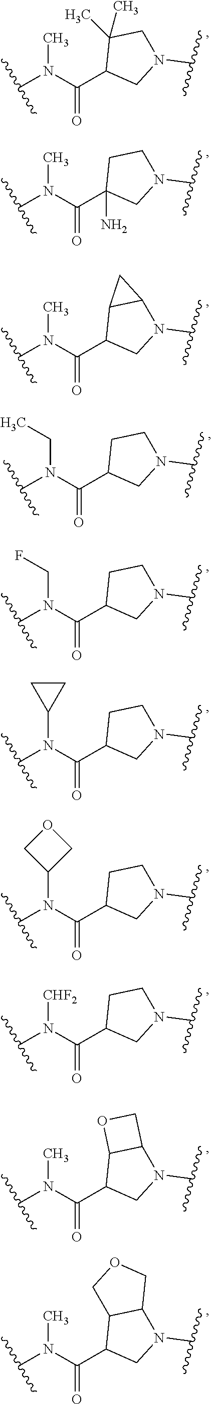

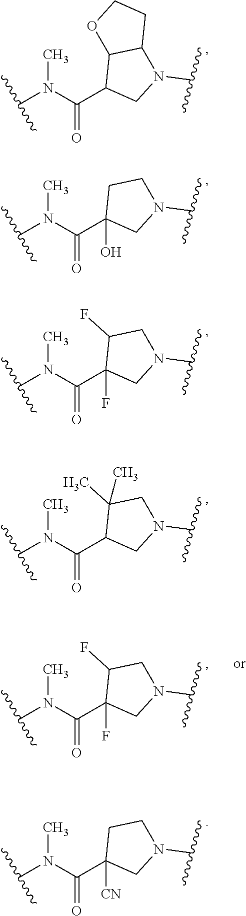

[74] The compound, or a pharmaceutically acceptable salt thereof, of paragraph [71], wherein the linker has the structure:

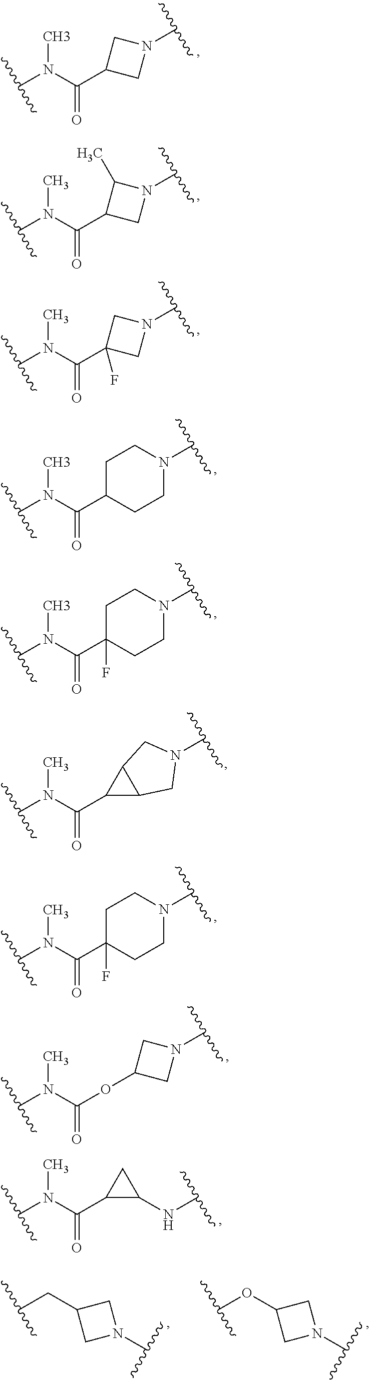

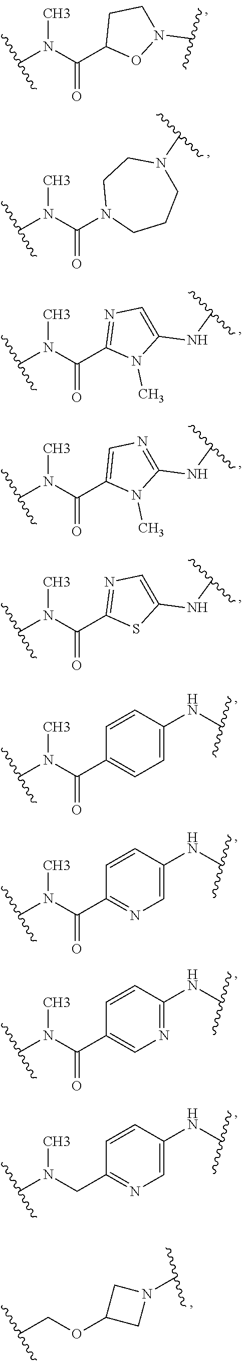

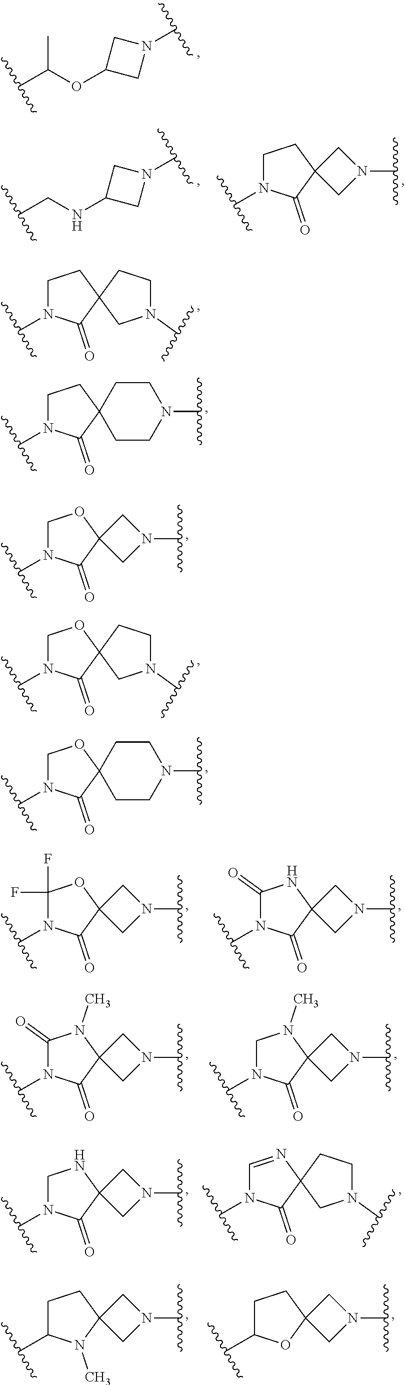

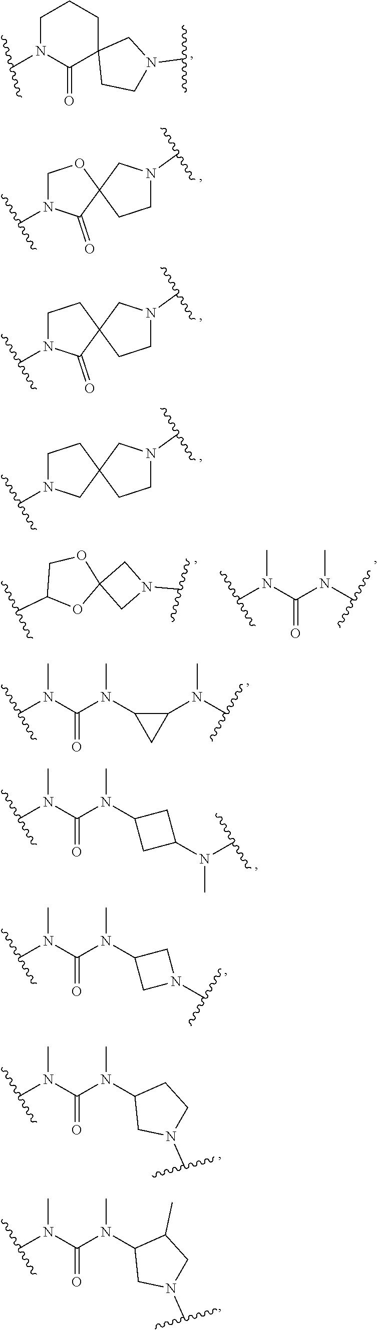

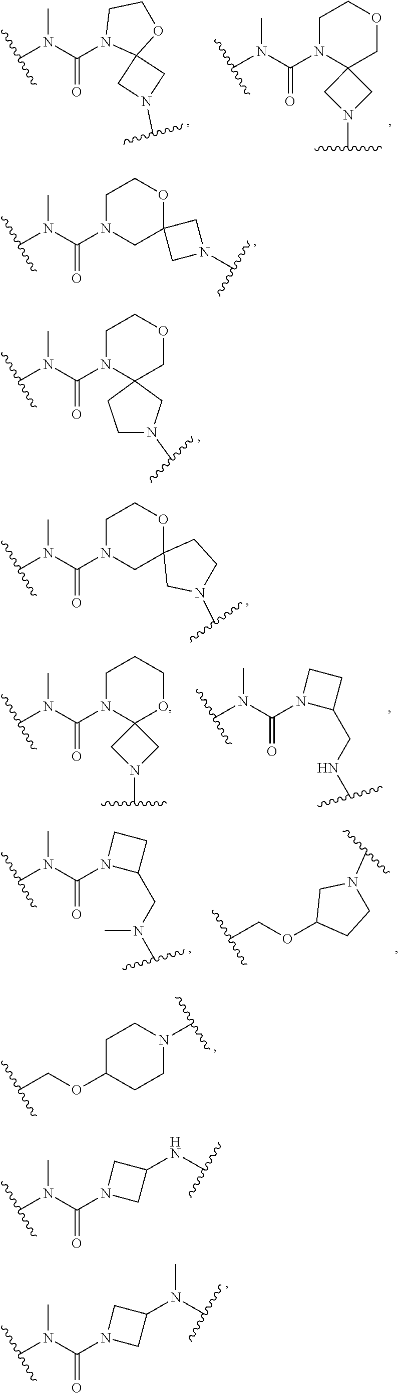

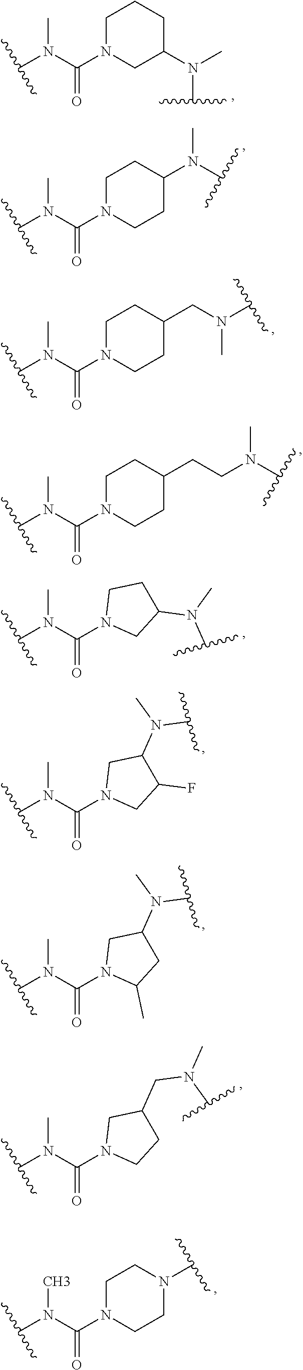

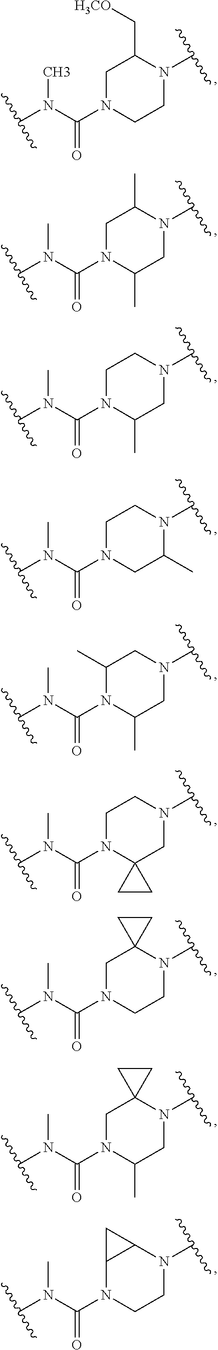

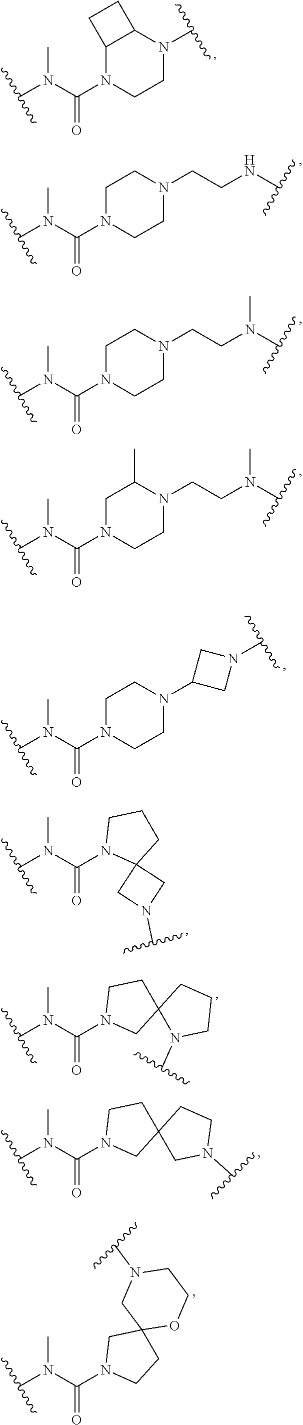

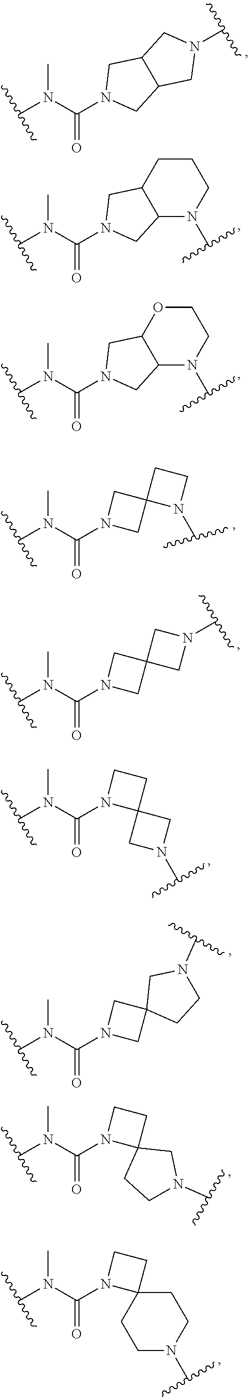

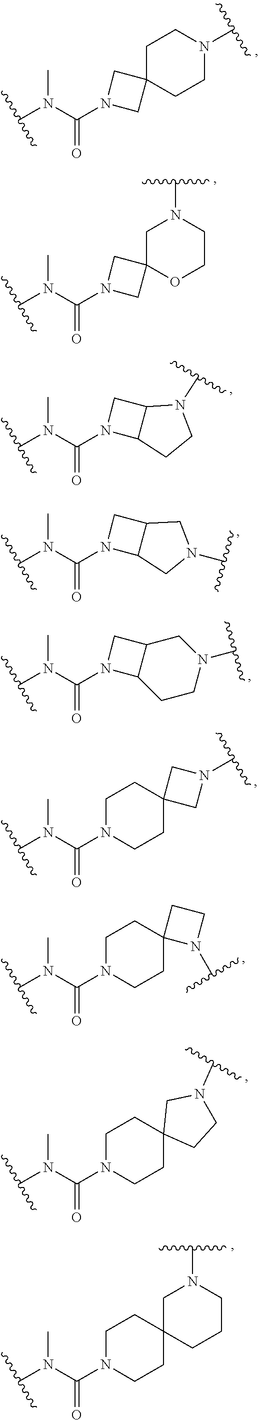

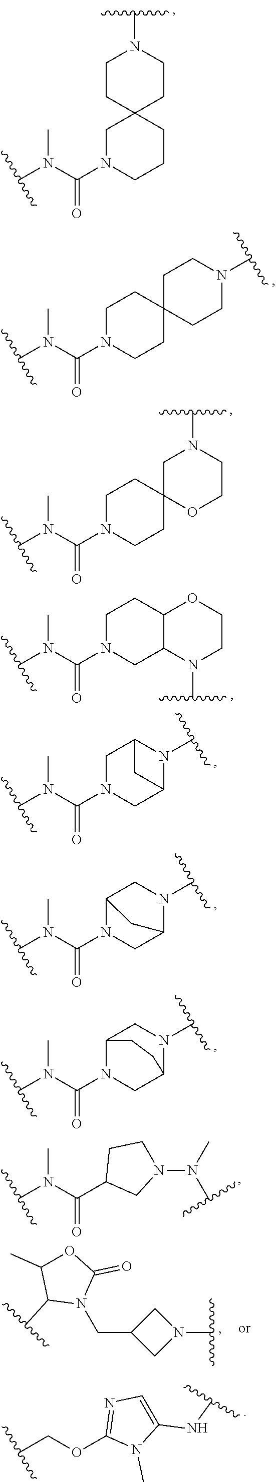

[75] The compound, or a pharmaceutically acceptable salt thereof, of any one of paragraphs [1] to [74], wherein W is a cross-linking group comprising a vinyl ketone.

[76]. The compound, or a pharmaceutically acceptable salt thereof, of paragraph [75], wherein W has the structure of Formula IIIa:

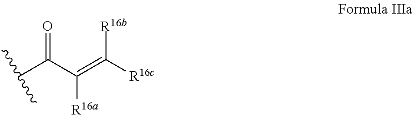

wherein R^{16a}, R^{16b}, and R^{16c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl.

[77] The compound, or a pharmaceutically acceptable salt thereof, of paragraph [76], wherein W is:

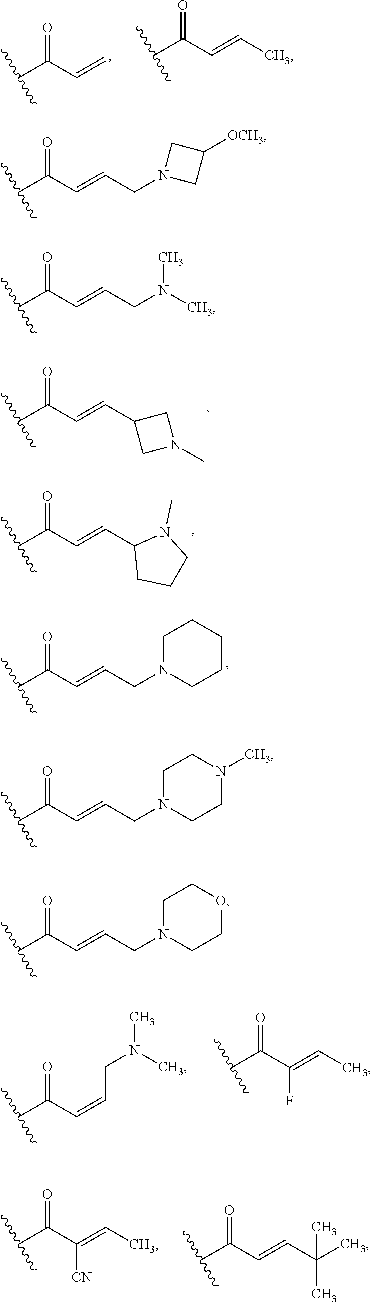

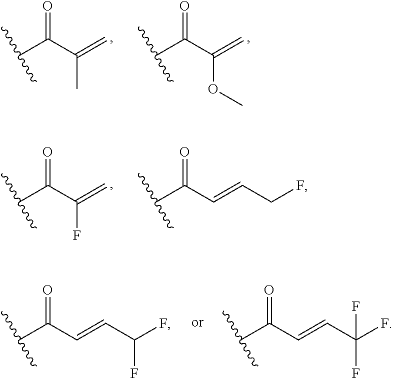

[78] The compound, or a pharmaceutically acceptable salt thereof, of any one of paragraphs [1] to [74], wherein W is a cross-linking group comprising an ynone.

[79] The compound, or a pharmaceutically acceptable salt thereof, of paragraph [78], wherein W has the structure of Formula IIIb:

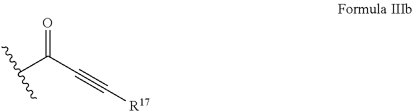

wherein R¹⁷ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated cycloalkyl, or a 4 to 7-membered saturated heterocycloalkyl.

[80] The compound, or a pharmaceutically acceptable salt thereof, of paragraph [79], wherein W is:

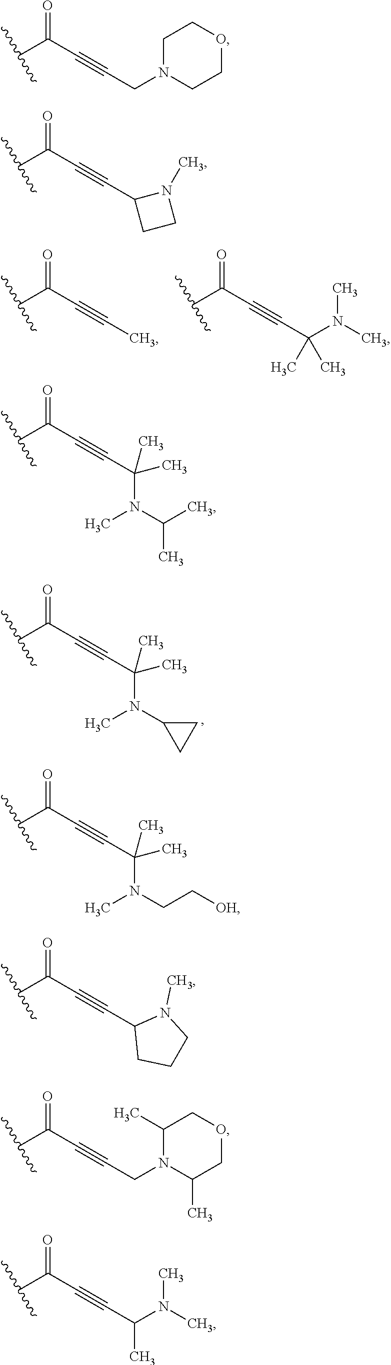

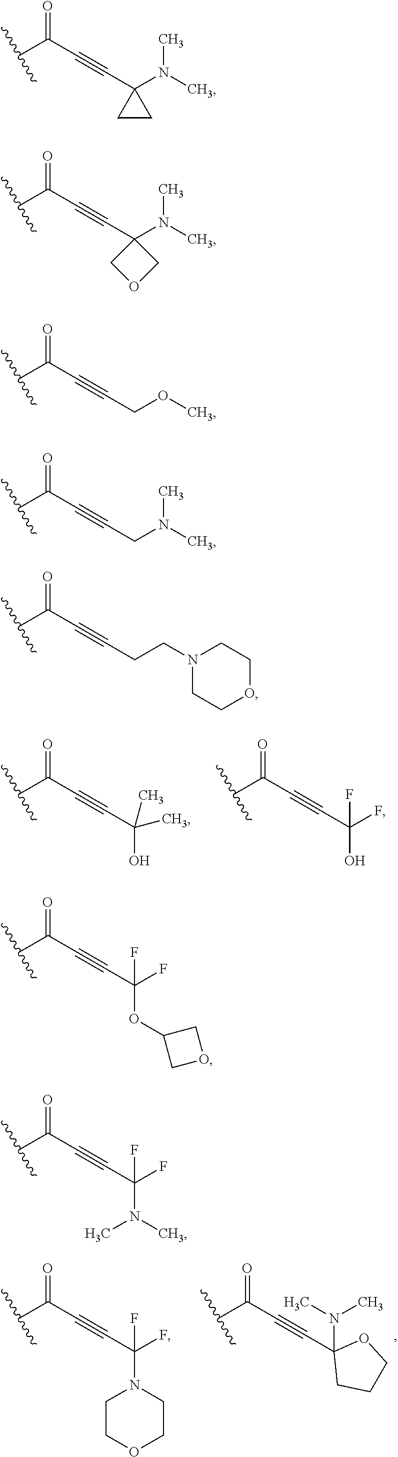

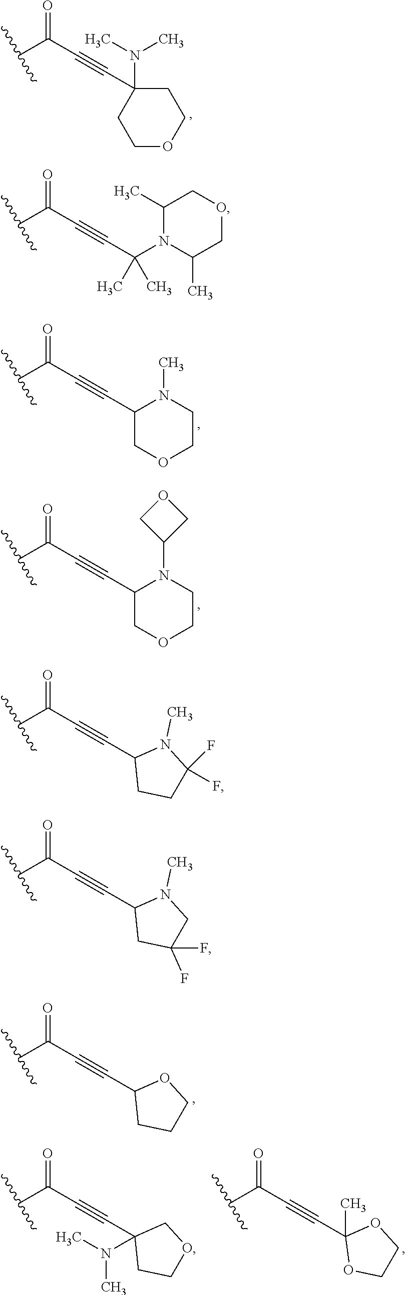

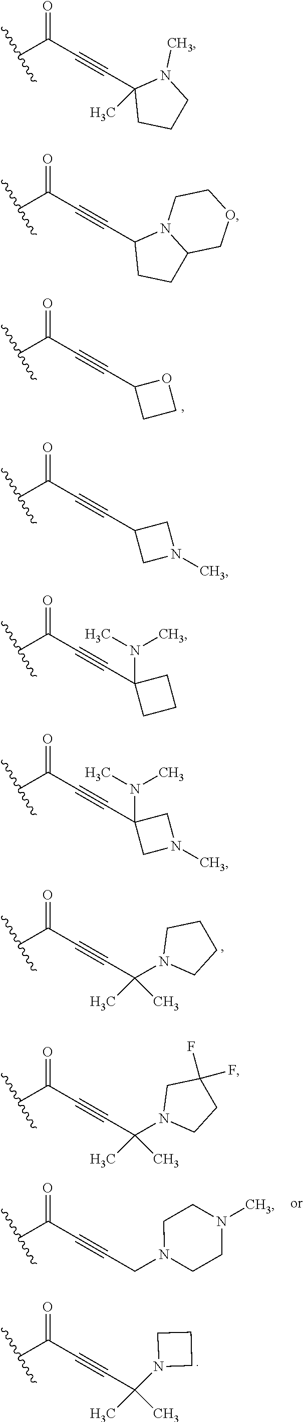

[81] The compound, or a pharmaceutically acceptable salt thereof, of any one of paragraphs [1] to [74], wherein W is a cross-linking group comprising a vinyl sulfone.

[82] The compound, or a pharmaceutically acceptable salt thereof, of paragraph [81] wherein W has the structure of Formula IIIc:

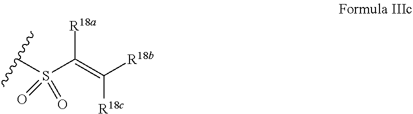

wherein R^{18a}, R^{18b}, and R^{18c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl.

[83] The compound, or a pharmaceutically acceptable salt thereof, of paragraph [82], wherein W is:

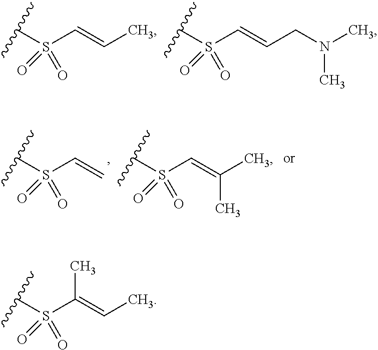

[84] The compound, or a pharmaceutically acceptable salt thereof, of any one of paragraphs [1] to [74], wherein W is a cross-linking group comprising a alkynyl sulfone.

[85] The compound, or a pharmaceutically acceptable salt thereof, of paragraph [84], wherein W has the structure of Formula IIId:

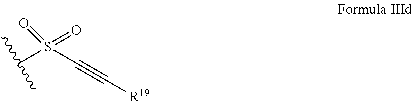

wherein R¹⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl.

[86] The compound, or a pharmaceutically acceptable salt thereof, of paragraph [85], wherein W is:

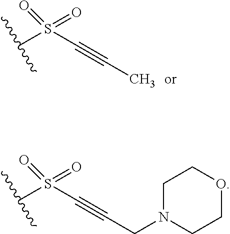

[87] The compound, or a pharmaceutically acceptable salt thereof, of any one of paragraphs [1] to [74], wherein W has the structure of Formula IIIe:

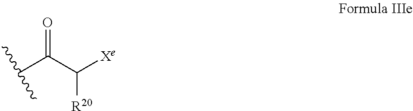

wherein X^{e} is a halogen; and

R²⁰ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl.

[88] A compound, or a pharmaceutically acceptable salt thereof, selected from Table 1 or Table 2.

[89] A pharmaceutical composition comprising a compound, or a pharmaceutically acceptable salt thereof, of any one of paragraphs [1] to [88], and a pharmaceutically acceptable excipient.

[90] A conjugate, or salt thereof, comprising the structure of Formula IV:

M-L-P Formula IV

wherein L is a linker;

P is a monovalent organic moiety; and

M has the structure of Formula V:

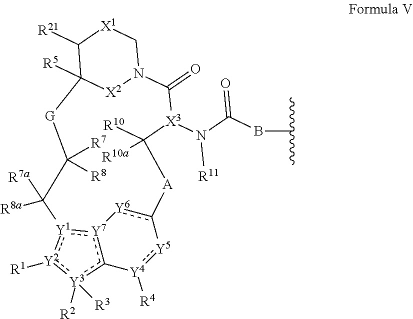

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is H, F, optionally substituted C₁-C₆ alkyl, optionally substituted C₂-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl; or

R⁹ and R^{9′}, combined with the atoms to which they are attached, form a 3 to 6-membered cycloalkyl or a 3 to 6-membered heterocycloalkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R²¹ is H or C₁-C₃ alkyl.

[91] The conjugate of paragraph [90], or salt thereof, wherein M has the structure of Formula Vd:

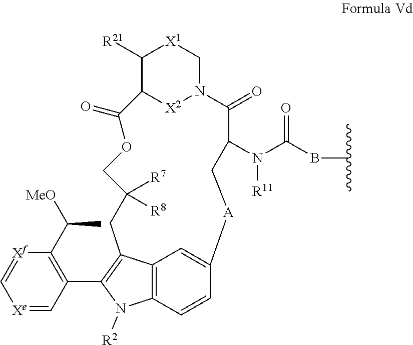

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

X¹ is optionally substituted C₁-C₂ alkylene, NR, O, or S(O)ₙ;

X² is O or NH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

R² is C₁-C₆ alkyl, C₁-C₆ fluoroalkyl, or 3 to 6-membered cycloalkyl;

R⁷ is C₁-C₃ alkyl;

R^{a} is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} and X^{f} are, independently, N or CH;

R¹¹ is hydrogen or C₁-C₃ alkyl; and

R²¹ is hydrogen or C₁-C₃ alkyl.

[92] The conjugate of paragraph [91], or salt thereof, wherein M has the structure of Formula Ve:

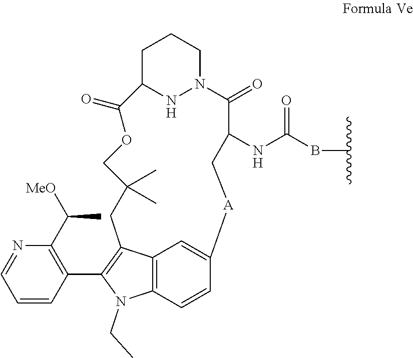

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

[93] The conjugate, or salt thereof, of any one of paragraphs [90] to [92], wherein the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A².

[94] The conjugate, or salt thereof, of any one of paragraphs [90] to [93], wherein the monovalent organic moiety is a protein.

[95] The conjugate, or salt thereof, of paragraph [94], wherein the protein is a Ras protein.

[96] The conjugate, or salt thereof, of paragraph [95], wherein the Ras protein is K-Ras G12C, K-Ras G13C, H-Ras G12C, H-Ras G13C, N-Ras G12C, or N-Ras G13C.

[97] The conjugate, or salt thereof, of any one of paragraphs [93] to [96], wherein the linker is bound to the monovalent organic moiety through a bond to a sulfhydryl group of an amino acid residue of the monovalent organic moiety.

[98] A method of treating cancer in a subject in need thereof, the method comprising administering to the subject a therapeutically effective amount of a compound, or a pharmaceutically acceptable salt thereof, of any one of paragraphs [1] to [88] or a pharmaceutical composition of paragraph [89].

[99] The method of paragraph [98], wherein the cancer is pancreatic cancer, colorectal cancer, non-small cell lung cancer, or endometrial cancer.

[100] The method of paragraph [98] or [99], wherein the cancer comprises a Ras mutation.

[101] The method of paragraph [100], wherein the Ras mutation is K-Ras G12C, K-Ras G13C, H-Ras G12C, H-Ras G13C, N-Ras G12C, or N-Ras G13C.

[102] A method of treating a Ras protein-related disorder in a subject in need thereof, the method comprising administering to the subject a therapeutically effective amount of a compound, or a pharmaceutically acceptable salt thereof, of any one of paragraphs [1] to [88] or a pharmaceutical composition of paragraph [89].

[103] A method of inhibiting a Ras protein in a cell, the method comprising contacting the cell with an effective amount of a compound, or a pharmaceutically acceptable salt thereof, of any one of paragraphs [1] to [88] or a pharmaceutical composition of paragraph [89].

[104] The method of paragraph [102] or [103], wherein the Ras protein is K-Ras G12C, K-Ras G13C, H-Ras G12C, H-Ras G13C, N-Ras G12C, or N-Ras G13C.

[105] The method of paragraph [103] or [104], wherein the cell is a cancer cell.

[106] The method of paragraph [105], wherein the cancer cell is a pancreatic cancer cell, a colorectal cancer cell, a non-small cell lung cancer cell, or an endometrial cancer cell.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:

| | |
|---|---|
| CH₂Cl₂, | Methylene chloride, Dichloromethane |
| DCM | |
| CH₃CN, | Acetonitrile |
| MeCN | |
| Cul | Copper (I) iodide |
| DIPEA | Diisopropylethyl amine |
| DMF | N, N-Dimethylformamide |
| EtOAc | Ethyl acetate |
| h | hour |
| H₂O | Water |
| HCl | Hydrochloric acid |
| K₃PO₄ | Potassium phosphate (tribasic) |
| MeOH | Methanol |
| Na₂SO₄ | Sodium sulfate |
| NMP | N-methyl pyrrolidone |
| Pd(dppf)Cl₂ | [1,1′-Bis(diphenylphosphino)ferrocene]dichloropalladium(II) |

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

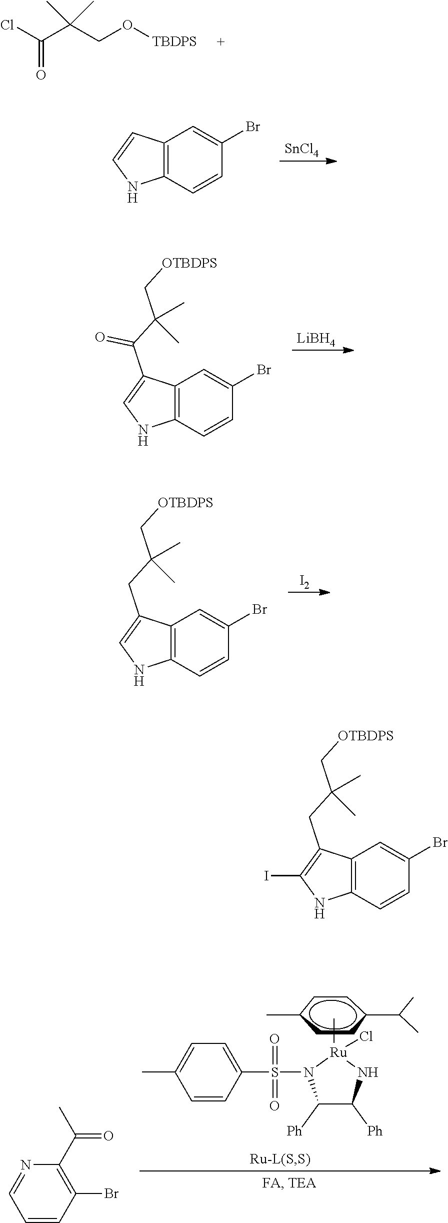

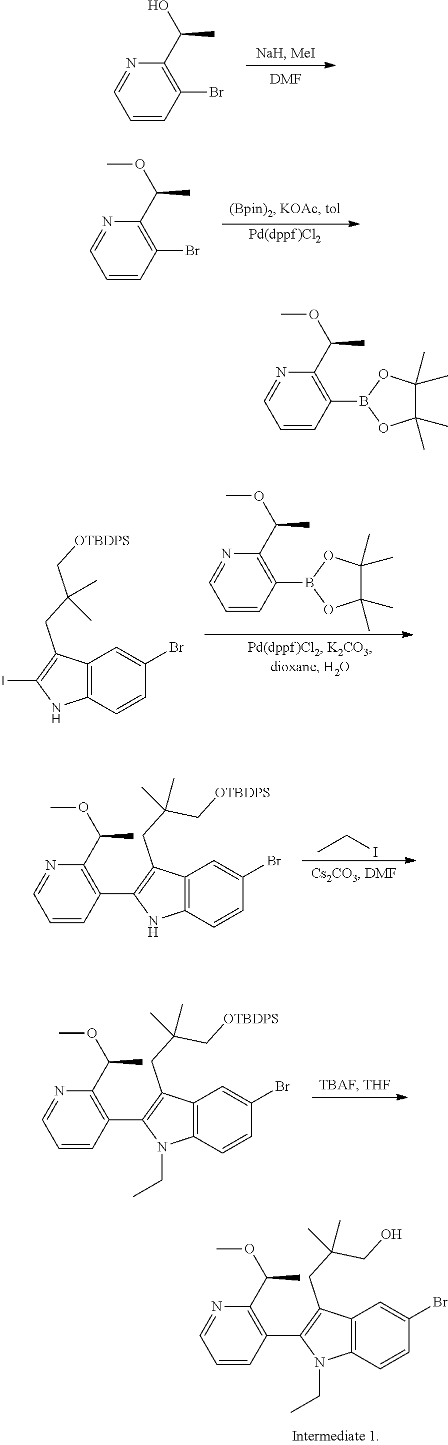

Step 1. To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (100 mL×4), dried over Na₂SO₄, and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z: [M+Na] calc'd for C₂₉H₃₂BrNO₂SiNa 556.1; found 556.3.

Step 2. To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered, and the filtrate concentrated under reduced pressure. The residue was diluted with DCM (50 mL), cooled to 10° C. and diludine (9.5 g, 37.4 mmol) and TsOH·H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z: [M+H] calc'd for C₂₉H₃₄BrNOSi 519.2; found 520.1; ¹H NMR (400 MHz, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

Step 3. To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄, and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHz, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

Step 4. To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl)ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄, and filtered. The filtrate was concentrated under reduced pressure and the residue was purified by silica gel column chromatography to give (1S)-1-(3-bromopyridin-2-yl)ethanol (100 g, 74% yield) a an oil. LCMS (ESI): m/z: [M+H] calc'd for C₇H₈BrNO 201.1; found 201.9.

Step 5. To a stirred mixture of (1S)-1-(3-bromopyridin-2-yl)ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI): m/z: [M+H] calc'd for C₈H₁₀BrNO 215.0; found 215.9.

Step 6. To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis(pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl)pyridine (100 g, 63% yield) as a semi-solid. LCMS (ESI): m/z: [M+H] calc'd for C₄H₂₂BNO₃: 263.2; found 264.1.

Step 7. To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl)pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol), and H₂O (280 mL) in portions. The mixture was heated to 85° C. and stirred for 4 h, then cooled, H₂O (5 L) added, and the mixture extracted with EtOAc (3×2 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI): m/z: [M+H] calc'd for C₃₇H₄₃BrN₂O₂Si 654.2; found 655.1.

Step 8. To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to rt and stirred for 16 h then H₂O (4 L) added and the mixture extracted with EtOAc (3×1.5 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z: [M+H] calc'd for C₃₉H₄₇BrN₂O₂Si 682.3; found 683.3.

Step 9. To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L), and extracted with EtOAc (3×1.5 L). The combined organic layers were washed with brine (2×1 L), dried over anhydrous Na₂SO₄, and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI): m/z: [M+H] calc'd for C₂₃H₂₉BrN₂O₂ 444.1; found 445.1.

### Intermediate 1. Alternative Synthesis through Fisher Indole Route

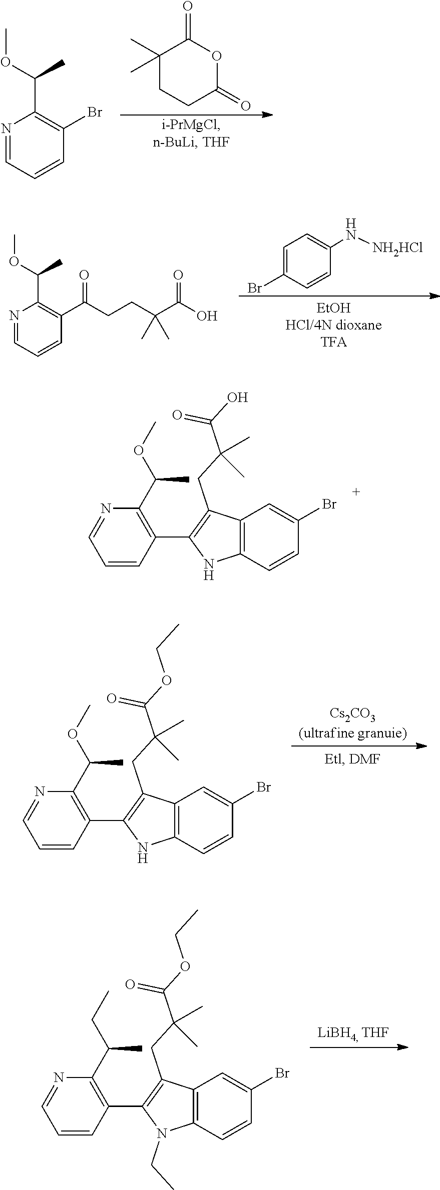

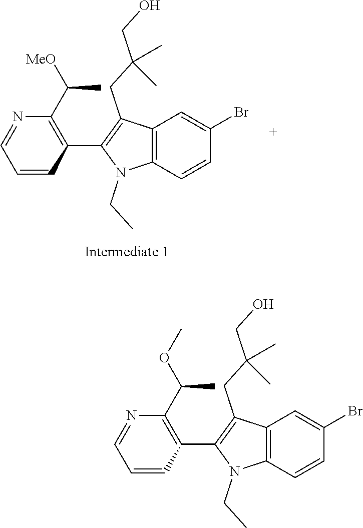

Step 1. To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure, and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z: [M+H] calc'd for C₁₅H₂₁NO₄ 279.2; found 280.1.

Step 2. To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl)hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure, and the residue was dissolved in TFA (0.78 L). The mixture was heated to 60° C. and stirred for 1.5 h, concentrated under reduced pressure, and the residue adjusted to pH ˜5 with saturated NaHCO₃, then extracted with EtOAc (3×1.5 L). The combined organic layers were dried over anhydrous Na₂SO₄, filtered, the filtrate concentrated under reduced pressure, and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (78 g, crude). LCMS (ESI): m/z: [M+H] calc'd for C₂₁H₂₃BrN₂O₃ 430.1 and C₂₃H₂₇BrN₂O₃ 458.1; found 431.1 and 459.1.

Step 3. To a mixture of 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. EtI (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄, and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI): m/z: [M+H] calc'd for C₂₅H₃₁BrN₂O₃ 486.2; found 487.2.

Step 4. To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄, and filtered. The filtrate was concentrated under reduced pressure and the residue was purified by silica gel column chromatography to give to two atropisomers (as single atropisomers) of 3-(5-bromo-1-ethyl-2-(2-((S)-1-methoxyethyl)pyridin-3-yl)-1H-indol-3-yl)-2,2-dimethylpropan-1-ol (60 g, 38% yield) and (40 g, 26% yield) both as solids. LCMS (ESI): m/z: [M+H] calc'd for C₂₃H₂₉BrN₂O₂ 444.1; found 445.2.

### Intermediate 2 and Intermediate 4. Synthesis of (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate

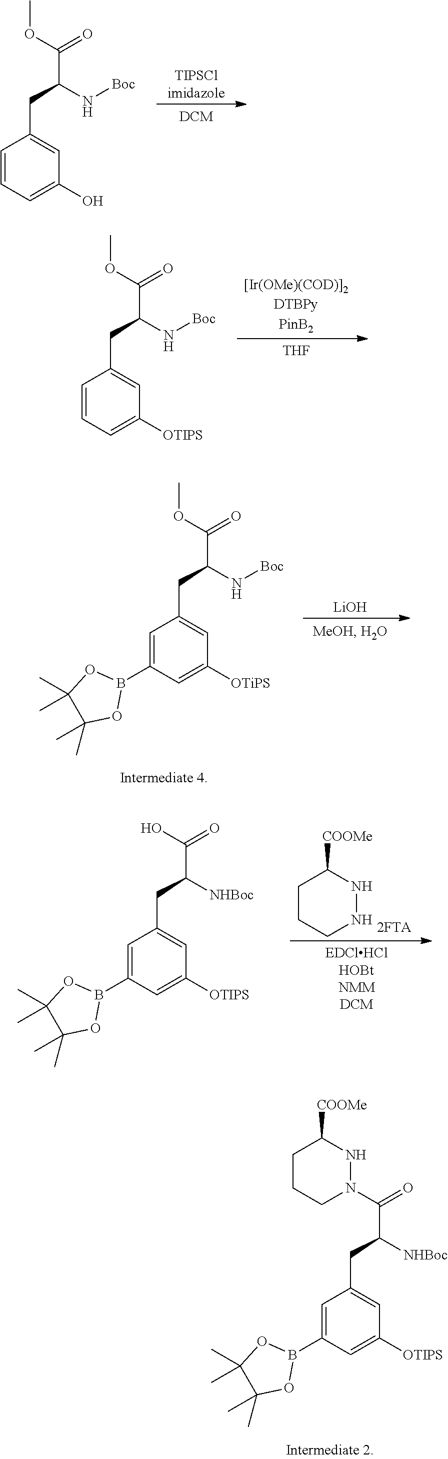

Step 1. To a mixture of (S)-methyl 2-(tert-butoxycarbonylamino)-3-(3-hydroxyphenyl)propanoate (10.0 g, 33.9 mmol) in DCM (100 mL) was added imidazole (4.6 g, 67.8 mmol) and TIPSCI (7.8 g, 40.7 mmol). The mixture was stirred at rt overnight then diluted with DCM (200 mL) and washed with H₂O (150 mL×3). The organic layer was dried over anhydrous Na₂SO₄, filtered, concentrated under reduced pressure, and the residue was purified by silica gel column chromatography to give (S)-methyl 2-(tert-butoxycarbonylamino)-3-(3-(triisopropylsilyloxy)phenyl)-propanoate (15 g, 98% yield) as an oil. LCMS (ESI): m/z: [M+Na] calc'd for C₂₄H₄₁NO₅SiNa 474.3; found 474.2.

Step 2. A mixture of (S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]₂ (1.1 g, 1.7 mmol), and 4-tert-butyl-2-(4-tert-butyl-2-pyridyl)pyridine (1.3 g, 5.0 mmol) was purged with Ar (×3), then THF (75 mL) was added and the mixture placed under an atmosphere of Ar and sealed. The mixture was heated to 80° C. and stirred for 16 h, concentrated under reduced pressure, and the residue was purified by silica gel column chromatography to give (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate (7.5 g, 78% yield) as a solid. LCMS (ESI): m/z: [M+Na] calc'd for C₃₀H₅₂BNO₇SiNa 600.4; found 600.4; ¹H NMR (300 MHz, CD₃OD) δ 7.18 (s, 1H), 7.11 (s, 1H), 6.85 (s, 1H), 4.34 (m, 1H), 3.68 (s, 3H), 3.08 (m, 1H), 2.86 (m, 1H), 1.41-1.20 (m, 26H), 1.20-1.01 (m, 22H), 0.98-0.79 (m, 4H).

Step 3. To a mixture of triisopropylsilyl (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to pH ˜5 with 1M HCl and extracted with EtOAc (250 mL×2). The combined organic layers were washed with brine (100 mL×3), dried over anhydrous Na₂SO₄, filtered, and the filtrate concentrated under reduced pressure to give (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (3.7 g, 95% yield), which was used directly in the next step without further purification. LCMS (ESI): m/z: [M+NH₄] calc'd for C₂₉H₅₀BNO₇SiNH₄ 581.4; found 581.4.

Step 4. To a mixture of methyl (S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (24 g, 42.6 mmol) in DCM (50 mL) then HOBt (1.21 g, 9.0 mmol) and EDCI HCl salt (12.9 g, 67.6 mmol). The mixture was warmed to rt and stirred for 16 h, then diluted with DCM (200 mL) and washed with H₂O (3×150 mL). The organic layer was dried over anhydrous Na₂SO, filtered, the filtrate concentrated under reduced pressure, and the residue was purified by silica gel column chromatography to give methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (22 g, 71/% yield) as an oil. LCMS (ESI): m/z: [M+H] calc'd for C₃₅H₆₀BN₃O₈Si 689.4; found 690.5.

### Intermediate 3. Synthesis of N—((S)-1-acryloylpyrrolidine-3-carbonyl)-N-methyl-L-valine

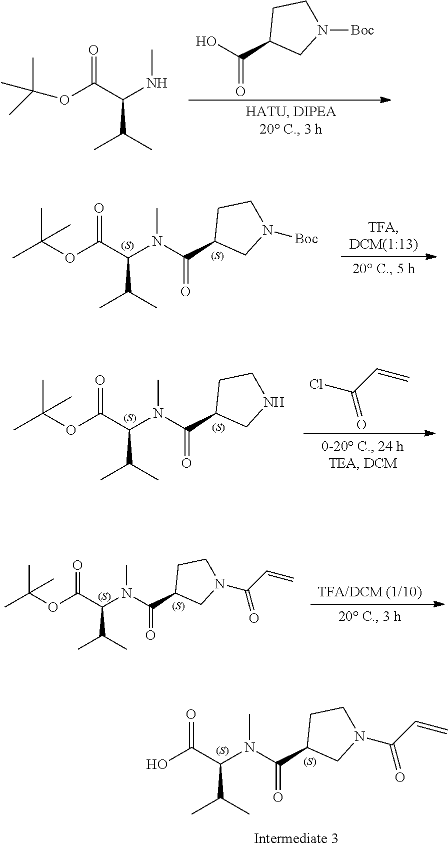

Step 1. To a mixture of (S)-1-(tert-butoxycarbonyl)pyrrolidine-3-carboxylic acid (2.2 g, 10.2 mmol) in DMF (10 mL) at rt was added HATU (7.8 g, 20.4 mmol) and DIPEA (5 mL). After stirring at rt for 10 min, tert-butyl methyl-L-valinate (3.8 g, 20.4 mmol) in DMF (10 mL) was added. The mixture was stirred at rt for 3 h, then diluted with DCM (40 mL) and H₂O (30 mL). The aqueous and organic layers were separated and the organic layer was washed with H₂O (3×30 mL), brine (30 mL), dried over anhydrous Na₂SO₄, and filtered. The filtrate was concentrated under reduced pressure and the residue was purified by silica gel column chromatography to give (S)-tert-butyl 3-(((S)-1-(tert-butoxy)-3-methyl-1-oxobutan-2-yl)(methyl)carbamoyl)pyrrolidine-1-carboxylate (3.2 g, 82% yield) as an oil. LCMS (ESI): m/z: [M+Na] calc'd for C₂₀H₃₆N₂O₅Na 407.3; found 407.2.

Step 2. A mixture of (S)-tert-butyl 3-(((S)-1-(tert-butoxy)-3-methyl-1-oxobutan-2-yl)(methyl)carbamoyl)pyrrolidine-1-carboxylate (3.2 g, 8.4 mmol) in DCM (13 mL) and TFA (1.05 g, 9.2 mmol) was stirred at rt for 5 h. The mixture was concentrated under reduced pressure to give (S)-tert-butyl 3-methyl-2-((S)—N-methylpyrrolidine-3-carboxamido)butanoate (2.0 g, 84% yield) as an oil. LCMS (ESI): m/z: [M+H] calc'd for C₁₅H₂₈N₂O₃ 284.2; found 285.2.

Step 3. To a mixture of (S)-tert-butyl 3-methyl-2-((S)—N-methylpyrrolidine-3-carboxamido)butanoate (600 mg, 2.1 mmol) in DCM (6 mL) at 0° C. was added TEA (342 mg, 3.36 mmol). After stirring at 0° C. for 10 mins, acryloyl chloride (284 mg, 3.2 mmol) in DCM (10 mL) was added. The mixture was warmed to rt and stirred for 24 h, then diluted with DCM (30 mL) and H₂O (30 mL). The aqueous and organic layers were separated and the organic layer was washed with H₂O (3×30 mL), brine (30 mL), dried over anhydrous Na₂SO₄, and filtered. The filtrate was concentrated under reduced pressure and the residue was purified by silica gel column chromatography to give tert-butyl N—((S)-1-acryloylpyrrolidine-3-carbonyl)-N-methyl-L-valinate (500 mg, 70% yield) as an oil.

Step 4. To a mixture of tert-butyl N—((S)-1-acryloylpyrrolidine-3-carbonyl)-N-methyl-L-valinate (100 mg, 0.29 mmol) in DCM (3.0 mL) at 15° C. was added TFA (0.3 mL). The mixture was warmed to rt and stirred for 5 h, then the mixture was concentrated under reduced pressure to give N—((S)-1-acryloylpyrrolidine-3-carbonyl)-N-methyl-L-valine (150 mg) as a solid. The crude product was used directly in the next step without further purification. LCMS (ESI): m/z: [M+H] calc'd for C₄H₂₂N₂O₄ 282.2; found 283.2.

### Intermediate 5. Synthesis of tert-butyl ((63S,4S)-11-ethyl-12-(2-((S)-1-methoxyethyl)pyridin-3-yl)-10,10-dimethyl-5,7-dioxo-25-((triisopropylsilyl)oxy)-61,62,63,64,65,66-hexahydro-11H-8-oxa-1(5,3)-indola-6(1,3)-pyridazina-2(1,3)-benzenacycloundecaphane-4-yl)carbamate

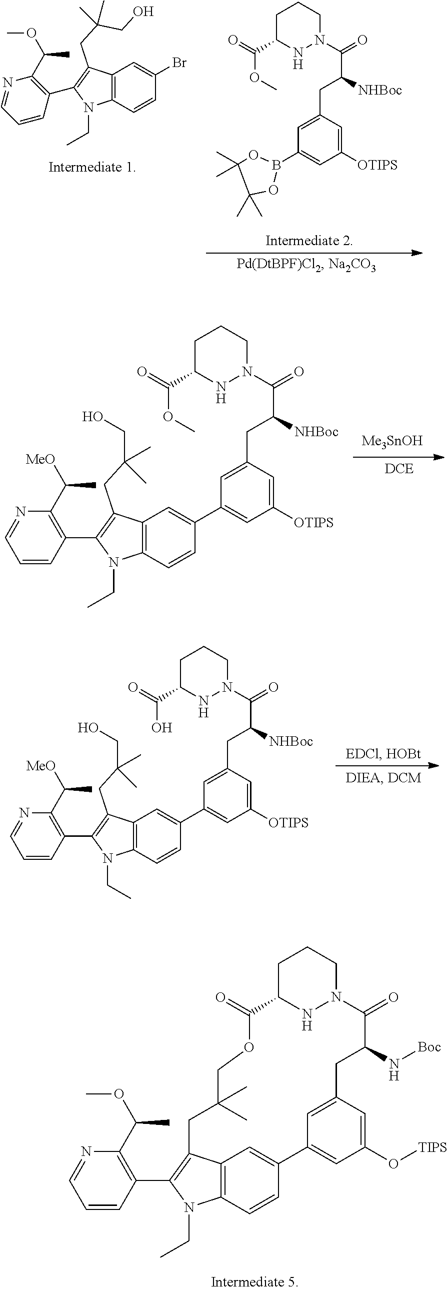

Step 1. To a stirred mixture of 3-(5-bromo-1-ethyl-2-[2-[(1S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in 1,4-dioxane (750 mL) at rt under an atmosphere of Ar was added Na₂CO₃ (17.9 g, 168.4 mmol), Pd(DtBPF)Cl₂ (4.39 g, 6.7 mmol), and H₂O (150.00 mL) in portions. The mixture was heated to 85° C. and stirred for 3 h, cooled, diluted with H₂O (2 L), and extracted with EtOAc (3×1 L). The combined organic layers were washed with brine (2×500 mL), dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 72% yield) as a solid. LCMS (ESI): m/z: [M+H] calc'd for C₅₂H₇₇N₅O₈Si 927.6; found 928.8.

Step 2. To a stirred mixture of methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at rt was added trimethyltin hydroxide (48.7 g, 269 mmol) in portion. The mixture was heated to 65° C. and stirred for 16 h, then filtered and the filter cake washed with DCM (3×150 mL). The filtrate was concentrated under reduced pressure to give (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g, crude), which was used directly in the next step without further purification. LCMS (ESI): m/z: [M+H] calc'd for C₅₁H₇₅N₅O₆Si 913.5; found 914.6.

Step 3. To a stirred mixture of (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (297 g, 2.3 mol), HOBT (51.7 g, 383 mmol) and EDCI (411 g, 2.1 mol) in portions. The mixture was warmed to rt and stirred for 16 h, then diluted with DCM (1 L), washed with brine (3×1 L), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (36 g, 42% yield) as a solid. LCMS (ESI): m/z: [M+H] calc'd for C₅₁H₇₃N₅O₇Si 895.5; found 896.5.

### Intermediate 6. Synthesis of tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate

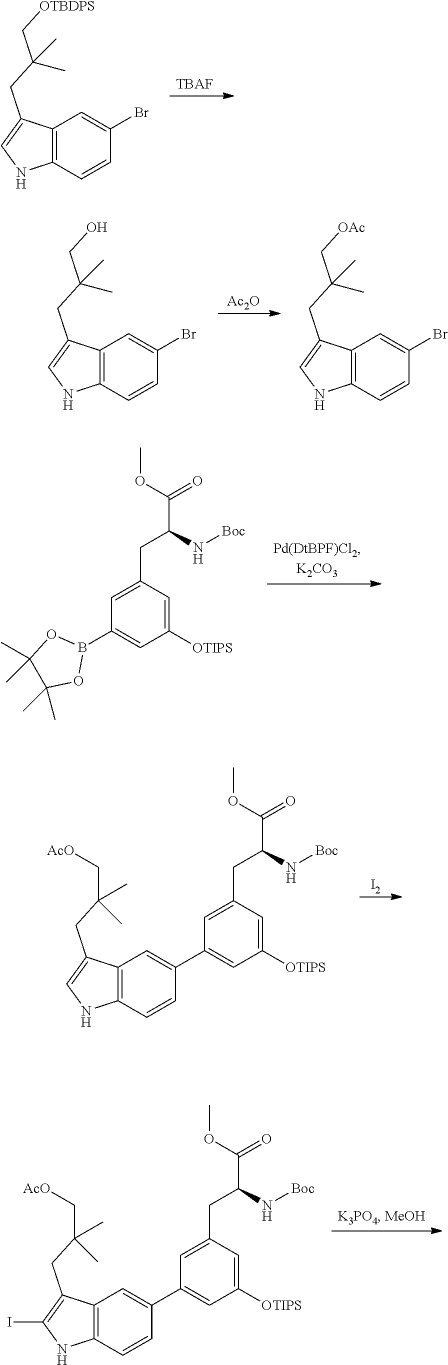

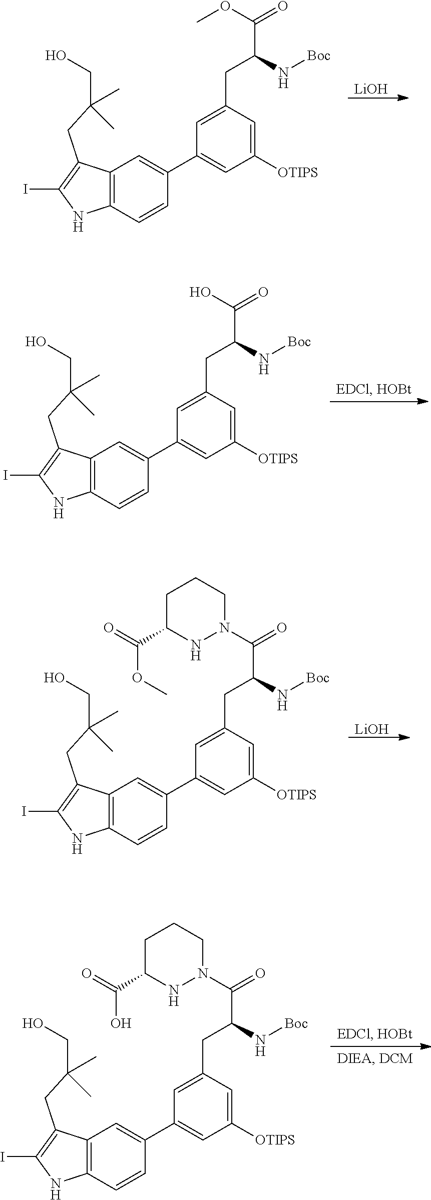

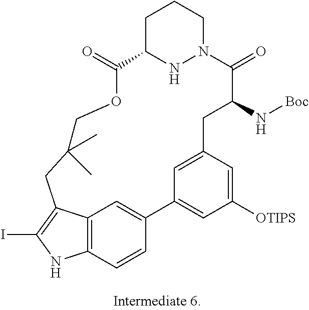

Step 1. This reaction was undertaken on 5-batches in parallel on the scale illustrated below.

Into a 2 L round-bottom flasks each were added 5-bromo-3-[3-[(tert-butyldiphenylsilyl)oxy]-2,2-dimethylpropyl]-1H-indole (100 g, 192 mmol) and TBAF (301.4 g, 1.15 mol) in THF (1.15 L) at rt. The resulting mixture was heated to 50° C. and stirred for 16 h, then the mixture was concentrated under reduced pressure. The combined residues were diluted with H₂O (5 L) and extracted with EtOAc (3×2 L). The combined organic layers were washed with brine (2×1.5 L), dried over anhydrous Na₂SO₄, and filtered. The filtrate was concentrated under reduced pressure and the residue was purified by silica gel column chromatography to give 3-(5-bromo-1H-indol-3-yl)-2,2-dimethylpropan-1-ol (310 g, crude) as a solid. LCMS (ESI): m/z: [M+H] calc'd for C₁₃H₁₆BrNO 281.0 and 283.0; found 282.1 and 284.1.

Step 2. This reaction was undertaken on two batches in parallel on the scale illustrated below.

To a stirred mixture of 3-(5-bromo-1H-indol-3-yl)-2,2-dimethylpropan-1-ol (135 g, 478 mmol) and TEA (145.2 g, 1.44 mol) in DCM (1.3 L) at 0° C. under an atmosphere of N₂ was added Ac₂O (73.3 g, 718 mmol) and DMAP (4.68 g, 38.3 mmol) in portions. The resulting mixture was stirred for 10 min at 0° C., then washed with H₂O (3×2 L). The organic layers from each experiment were combined and washed with brine (2×1 L), dried over anhydrous Na₂SO₄, and filtered. The filtrate was concentrated under reduced pressure and the residue was purified by column chromatography to give 3-(5-bromo-1H-indol-3-yl)-2,2-dimethylpropyl acetate (304 g, 88% yield) as a solid. ¹H NMR (400 MHz, DMSO-d₆) δ 11.16-11.11 (m, 1H), 7.69 (d, J=2.0 Hz, 1H), 7.32 (d, J=8.6 Hz, 1H), 7.19-7.12 (m, 2H), 3.69 (s, 2H), 2.64 (s, 2H), 2.09 (s, 3H), 0.90 (s, 6H).

Step 3. This reaction was undertaken on four batches in parallel on the scale illustrated below.

Into a 2 L round-bottom flasks were added methyl (2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoate (125 g, 216 mmol), 1,4-dioxane (1 L), H₂O (200 mL), 3-(5-bromo-1H-indol-3-yl)-2,2-dimethylpropyl acetate (73.7 g, 227 mmol), K₂CO₃ (59.8 g, 433 mmol), and Pd(DtBPF)Cl₂ (7.05 g, 10.8 mmol) at rt under an atmosphere of Ar. The resulting mixture was heated to 65° C. and stirred for 2 h, then diluted with H₂O (10 L) and extracted with EtOAc (3×3 L). The combined organic layers were washed with brine (2×2 L), dried over anhydrous Na₂SO₄, and filtered. The filtrate was concentrated under reduced pressure and the residue was purified by column chromatography to give methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate (500 g, 74% yield) as an oil. LCMS (ESI): m/z: [M+Na] calc'd for C₃₉H₅₈N₂O₇SiNa 717.4; found 717.3.

Step 4. This reaction was undertaken on three batches in parallel on the scale illustrated below.

To a stirred mixture of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate (150 g, 216 mmol) and NaHCO₃ (21.76 g, 259 mmol) in THF (1.5 L) was added AgOTf (66.5 g, 259 mmol) in THF dropwise at 0° C. under an atmosphere of nitrogen. 12 (49.3 g, 194 mmol) in THF was added dropwise over 1 h at 0° C. and the resulting mixture was stirred for an additional 10 min at 0° C. The combined experiments were diluted with aqueous Na₂S₂O₃ (5 L) and extracted with EtOAc (3×3 L). The combined organic layers were washed with brine (2×1.5 L), dried over anhydrous Na₂SO₄, and filtered. The filtrate was concentrated under reduced pressure and the residue was purified by column chromatography to give methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate (420 g, 71% yield) as an oil. LCMS (ESI): m/z: [M+Na] calc'd for C₃₉H₅₇IN₂O₇SiNa, 843.3; found 842.9.

Step 5. This reaction was undertaken on three batches in parallel on the scale illustrated below.

To a 2 L round-bottom flask were added methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate (140 g, 171 mmol), MeOH (1.4 L) and K₃PO₄ (108.6 g, 512 mmol) at 0° C. The mixture was warmed to rt and stirred for 1 h, then the combined experiments were diluted with H₂O (9 L) and extracted with EtOAc (3×3 L). The combined organic layers were washed with brine (2×2 L), dried over anhydrous Na₂SO₄, filtered, and the filtrate was concentrated under reduced pressure to give methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate (438 g, crude) as a solid. LCMS (ESI): m/z: [M+Na] calc'd for C₃₇H₅₅IN₂O₆SiNa 801.3; found 801.6.

Step 6. This reaction was undertaken on three batches in parallel on the scale illustrated below.

To a stirred mixture of methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate (146 g, 188 mmol) in THF (1.46 L) was added LiOH (22.45 g, 937 mmol) in H₂O (937 mL) dropwise at 0° C. The resulting mixture was warmed to rt and stirred for 1.5 h [note: LCMS showed 15% de-TIPS product]. The mixture was acidified to pH 5 with 1M HCl (1M) and the combined experiments were extracted with EtOAc (3×3 L). The combined organic layers were washed with brine (2×2 L), dried over anhydrous Na₂SO₄, filtered, and the filtrate was concentrated under reduced pressure to give (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid (402 g, crude) as a solid. LCMS (ESI): m/z: [M+Na] calc'd for C₃₆H₅₃IN₂O₆SiNa 787.3; found 787.6.

Step 7. To a stirred mixture of (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid (340 g, 445 mmol) and methyl (3S)-1,2-diazinane-3-carboxylate (96.1 g, 667 mmol) in DCM (3.5 L) was added NMM (225 g, 2.2 mol), EDCI (170 g, 889 mmol), and HOBT (12.0 g, 88.9 mmol) portionwise at 0° C. The mixture was warmed to rt and stirred for 16 h, then washed with H₂O (3×2.5 L), brine (2×1 L), dried over anhydrous Na₂SO₄ and filtered. The filtrate was concentrated under reduced pressure and the residue was purified by column chromatography to give methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (310 g, 62% yield) as an oil. LCMS (ESI): m/z: [M+H] calc'd for C₄₂H₆₁IN₄O₇Si 890.4; found 890.8.

Step 8. This reaction was undertaken on three batches in parallel on the scale illustrated below.

To a stirred mixture of methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (85.0 g, 95.4 mmol) in THF (850 mL) each added LiOH (6.85 g, 286 mmol) in H₂O (410 mL) dropwise at 0° C. under an atmosphere of N₂. The mixture was stirred at 0° C. for 1.5 h [note: LCMS showed 15% de-TIPS product], then acidified to pH 5 with 1M HCl and the combined experiments extracted with EtOAc (3×2 L). The combined organic layers were washed with brine (2×1.5 L), dried over anhydrous Na₂SO₄, filtered, and the filtrate was concentrated under reduced pressure to give (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (240 g, crude) as a solid. LCMS (ESI): m/z: [M+H] calc'd for C₄₁H₆₁IN₄O₇Si 876.3; found 877.6.

Step 9.

This reaction was undertaken on two batches in parallel on the scale illustrated below.

To a stirred mixture of (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (120 g, 137 mmol) in DCM (6 L) was added DIPEA (265 g, 2.05 mol), EDCI (394 g, 2.05 mol), and HOBT (37 g, 274 mmol) in portions at 0° C. under an atmosphere of N₂. The mixture was warmed to rt and stirred overnight, then the combined experiments were washed with H₂O (3×6 L), brine (2×6 L), dried over anhydrous Na₂SO₄, and filtered. After filtration, the filtrate was concentrated under reduced pressure. The filtrate was concentrated under reduced pressure and the residue was purified by column chromatography to give tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (140 g, 50% yield) as a solid. LCMS (ESI): m/z [M+H] calc'd for C₄₁H₅₉IN₄O₆Si 858.9; found 858.3.

### Intermediate 7. Synthesis of (S)-methyl 2-((tert-butoxycarbonyl)amino)-3-(4-(4,4,5,5-tetramethyl-1,3,2-dioxaborolan-2-yl)pyridin-2-yl)propanoate

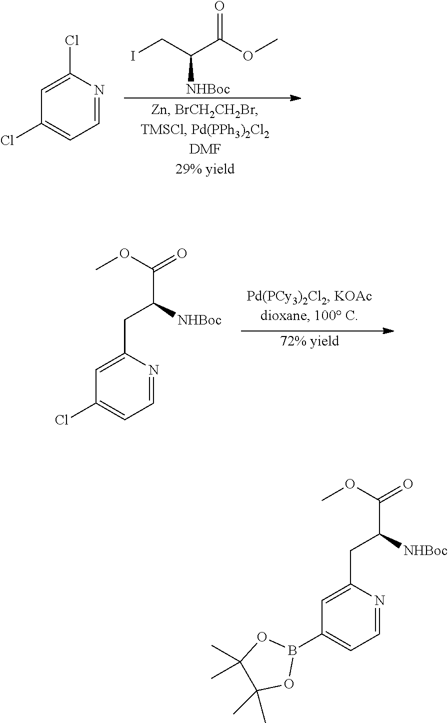 [SMILES: Clc1ccnc(Cl)c1 ; COC(=O)[C@H](CI)NC(=O)OC(C)(C)C ; COC(=O)[C@H](Cc1cc(Cl)ccn1)NC(=O)OC(C)(C)C ; COC(=O)[C@H](Cc1cc(B2OC(C)(C)C(C)(C)O2)ccn1)NC(=O)OC(C)(C)C]

Step 1. Zn dust (28 g, 428 mmol) was added to a 1 L, three necked, round bottomed flask, purged with N₂, and heated with a heat gun for 10 min under vacuum. The mixture was cooled to rt, and a solution of 1,2-dibromoethane (1.85 mL, 21.5 mmol) in DMF (90 mL) was added dropwise over 10 min. The mixture was heated at 90° C. for 30 min and re-cooled to rt. TMSCI (0.55 mL, 4.3 mmol) was added, and the mixture was stirred for 30 min at rt, then a mixture of (R)-methyl 2-((tert-butoxycarbonyl)amino)-3-iodopropanoate (22.5 g, 71.4 mmol) in DMF (200 mL) was added dropwise over a period of 10 min. The mixture was heated at 35° C. and stirred for 2 h, then cooled to rt, and 2,4-dichloropyridine (16 g, 109 mmol) and Pd(PPh₃)₂Cl₂ (4 g, 5.7 mmol) added. The mixture was heated at 45° C. and stirred for 2 h, cooled, and filtered, then H₂O (1 L) and EtOAc (0.5 L) were added to the filtrate. The organic and aqueous layers were separated, and the aqueous layer was extracted with EtOAc (2×500 mL). The organic layers were dried over anhydrous Na₂SO₄, filtered, and concentrated under reduced pressure. The crude residue was purified by silica gel column chromatography to give (S)-methyl 2-((tert-butoxycarbonyl)amino)-3-(4-chloropyridin-2-yl)propanoate (6.5 g, 29/c yield) as a solid. LCMS (ESI): m/z [M+H] calc'd for C₁₄H₁₉ClN₂O₄ 314.1; found 315.1.

Step 2. To a mixture of (S)-methyl 2-((tert-butoxycarbonyl)amino)-3-(4-chloropyridin-2-yl)propanoate (6.5 g, 20.6 mmol) in 1,4-dioxane (80 mL) at rt under an atmosphere of N₂ was added bis(pinacolato)diboron (6.3 g, 24.7 mmol), KOAc (8.1 g, 82.4 mmol), and Pd(PCy₃)₂Cl₂ (1.9 g, 2.5 mmol). The mixture was heated to 100° C. and stirred for 3 h, then H₂O (100 ml) added and the mixture extracted with EtOAc (3×200 mL). The organic layers were combined, washed with brine (2×100 mL), dried over anhydrous Na₂SO₄, and filtered. The filtrate was concentrated under reduced pressure and the residue was purified by silica gel column chromatography to give (S)-methyl 2-((tert-butoxycarbonyl)amino)-3-(4-(4,4,5,5-tetramethyl-1,3,2-dioxaborolan-2-yl)pyridin-2-yl)propanoate (6 g, 72% yield) as a solid. LCMS (ESI): m/z: [M+H] calc'd for C₂₀H₃₁BN₂O₆ 406.2; found 407.3.

### Synthesis of Intermediate 8

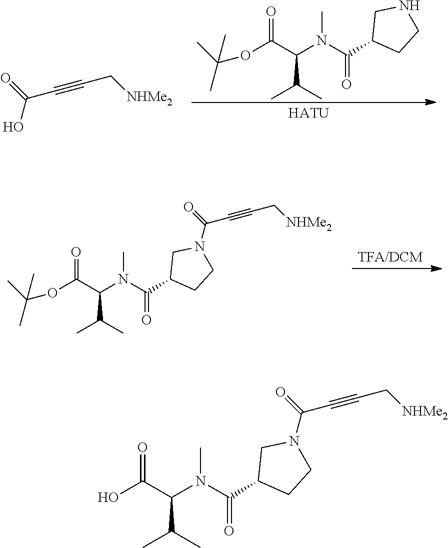

Step 1. To a mixture of 4-(dimethylamino)but-2-ynoic acid (900 mg, 7.0 mmol) in DMF (20 mL) at −5° C. was added tert-butyl N-methyl-N—((S)-pyrrolidine-3-carbonyl)-L-valinate (1.0 g, 3.5 mmol), DIPEA (2.2 g, 17.6 mmol) and HATU (2.7 g, 7.0 mmol) in portions. The mixture was stirred between −5 to 5° C. for 1 h, then diluted with EtOAc (100 mL) and ice-H₂O (100 mL). The aqueous and organic layers were separated and the organic layer was washed with H₂O (3×100 mL), brine (100 mL), dried over anhydrous Na₂SO₄, and filtered. The filtrate was concentrated under reduced pressure and the residue was purified by silica gel column chromatography to give tert-butyl N—((S)-1-(4-(dimethylamino)but-2-ynoyl)pyrrolidine-3-carbonyl)-N-methyl-L-valinate (900 mg, 55% yield) as an oil. LCMS (ESI): m/z: [M+H] calc'd for C₂₁H₃₅N₃O₄ 393.5; found 394.3.

Step 2. To a mixture of tert-butyl N—((S)-1-(4-(dimethylamino)but-2-ynoyl)pyrrolidine-3-carbonyl)-N-methyl-L-valinate (260 mg, 0.66 mmol) in DCM (6 mL) was added TFA (3 mL) at rt. The mixture was stirred at rt for 2 h, then the solvent was concentrated under reduced pressure to give (2S)-2-{1-[(3S)-1-[4-(dimethylamino)but-2-ynoyl]pyrrolidin-3-yl]-N-methylformamido}-3-methylbutanoic acid (280 mg) as an impure oil. The crude product was used directly in the next step without further purification. LCMS (ESI): m/z: [M+H] calc'd for C₁₇H₂₇N₃O₄ 337.2; found 338.3.

### Synthesis of Intermediate 9

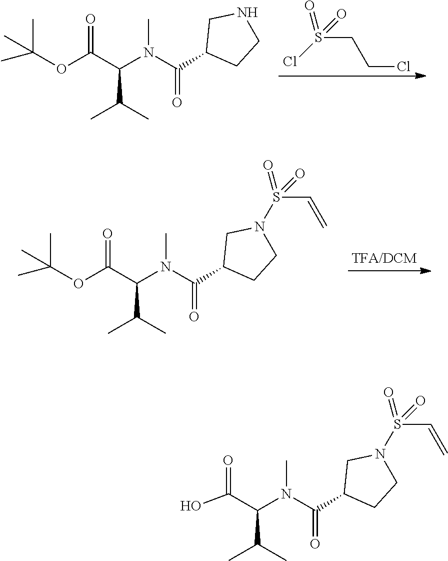

Step 1. To a mixture of tert-butyl N-methyl-N—((S)-pyrrolidine-3-carbonyl)-L-valinate (500 mg, 1.8 mmol) in DCM (8 mL) at 5° C. was added TEA (533 mg, 5.3 mmol) followed by dropwise addition of 2-chloroethane-1-sulfonyl chloride (574 mg, 3.5 mmol) in DCM (2 mL) The mixture was stirred at 5° C. for 1 h, then diluted with H₂O (20 mL) and extracted with EtOAC (3×10 mL). The combined organic layers were washed with brine (10 mL), dried over anhydrous Na₂SO₄, and filtered. The filtrate was concentrated under reduced pressure and the residue was purified by silica gel column chromatography to give tert-butyl N-methyl-N—((S)-1-(vinylsulfonyl)pyrrolidine-3-carbonyl)-L-valinate (300 mg, 45% yield) as an oil. LCMS (ESI): m/z: [M+H] calc'd for C₁₇H₃₀N₂O₅S 374.2; found 375.2.

Step 2. To a mixture of tert-butyl N-methyl-N—((S)-1-(vinylsulfonyl)pyrrolidine-3-carbonyl)-L-valinate (123 mg, 0.33 mmol) in DCM (3 mL) at rt was added TFA (1 mL). The mixture was stirred at rt for 1 h, then concentrated under reduced pressure to give N-methyl-N—((S)-1-(vinylsulfonyl)pyrrolidine-3-carbonyl)-L-valine (130 mg, crude) as a solid, which was used directly in the next step without further purification. LCMS (ESI): m/z: [M+H] calc'd for C₁₃H₂₂N₂O₅S 318.1; found 319.1.

### Synthesis of Intermediate 10

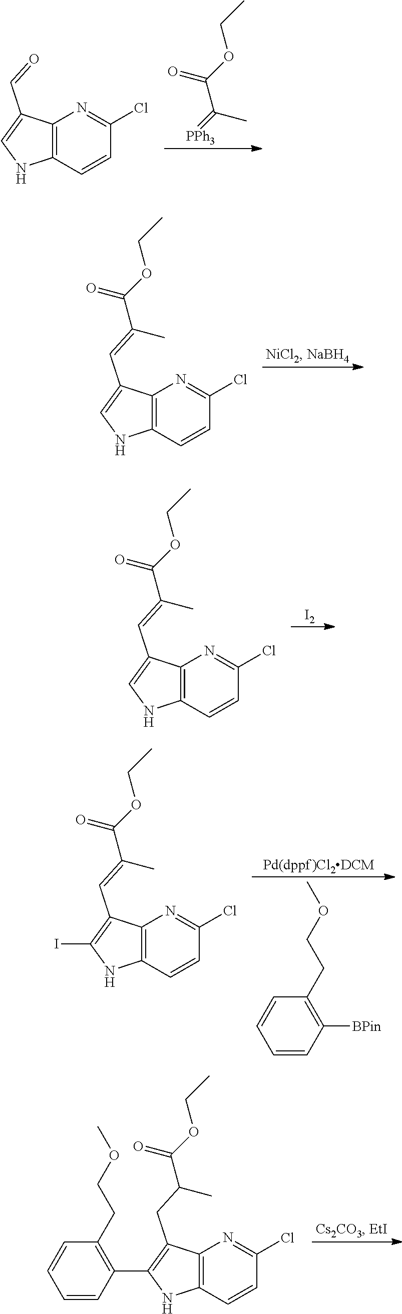

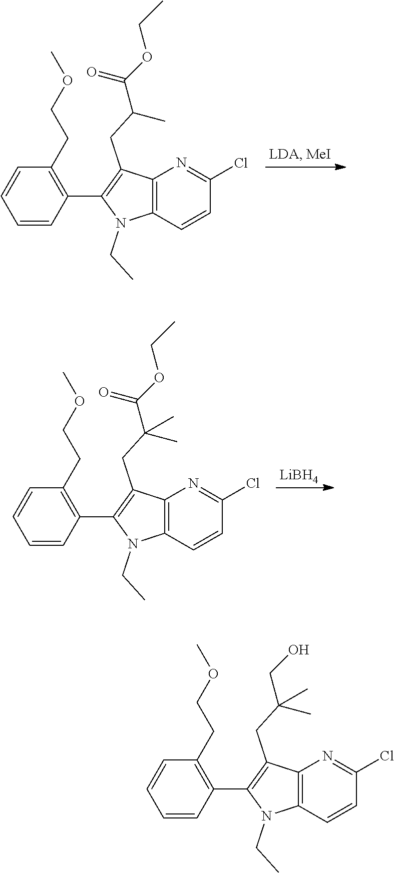

Step 1. A mixture of 5-chloro-1H-pyrrolo[3,2-b]pyridine-3-carbaldehyde (8.5 g, 47.1 mmol) and ethyl 2-(triphenylphosphoranylidene)propionate (2.56 g, 70.7 mmol) in 1,4-dioxane (120 mL) was stirred at reflux for 4 h, then concentrated under reduced pressure. EtOAc (200 mL) was added and the mixture was washed with brine, dried over Na₂SO₄, and filtered. The filtrate was concentrated under reduced pressure and the residue was purified by silica gel column chromatography to give ethyl (E)-3-(5-chloro-1H-pyrrolo[3,2-b]pyridin-3-yl)-2-methylacrylate (7.5 g, 60% yield) as a solid. LCMS (ESI): m/z: [M+4H] calc'd for C₁₃H₁₃ClN₂O₂ 264.1; found 265.1.

Step 2. To a mixture of ethyl (E)-3-(5-chloro-1H-pyrrolo[3,2-b]pyridin-3-yl)-2-methylacrylate (7.5 g, 28.3 mmol) and NiCl₂ (4.8 g, 28.3 mmol) in 1:1 THF/MeOH (300 mL) was added NaBH₄ (21.5 g, 566 mmol) in 20 portions every 25 minutes. After complete addition, the mixture was stirred at rt for 30 min, then diluted with EtOAc (500 mL) and washed with brine, dried over Na₂SO₄, and filtered. The filtrate was concentrated under reduced pressure and the residue was purified by silica gel column chromatography to give ethyl 3-(5-chloro-1H-pyrrolo[3,2-b]pyridin-3-yl)-2-methylpropanoate (3.4 g, 45% yield) as a solid. LCMS (ESI): m/z: [M+H] calc'd for C₁₃H₁₅ClN₂O₂ 266.1; found 267.1.

Step 3. To a mixture of ethyl 3-(5-chloro-1H-pyrrolo[3,2-b]pyridin-3-yl)-2-methylpropanoate (7.0 g, 26.2 mmol) and AgOTf (6.7 g, 26.2 mmol) in THF (50 mL) at 0° C. was added 12 (6.65 g, 26.2 mol). The mixture was stirred at 0° C. for 30 mi then diluted with EtOAc (100 mL), washed with Na₂SO₃ (50 mL), brine (50 mL), dried over Na₂SO₄, and filtered. The filtrate was concentrated under reduced pressure and the residue was purified by silica gel column chromatography to give ethyl 3-(5-chloro-2-iodo-1H-pyrrolo[3,2-b]pyridin-3-yl)-2-methylpropanoate (6 g, 58% yield) as white solid. LCMS (ESI): m/z: [M+H] calc'd for C₁₃H₁₄ClIN₂O₂ 392.0; found 393.0.

Step 4. To a mixture of ethyl 3-(5-chloro-2-iodo-1H-pyrrolo[3,2-b]pyridin-3-yl)-2-methylpropanoate (6.0 g, 15.3 mmol) and 2-(2-(2-methoxyethyl)phenyl)-4,4,5,5-tetramethyl-1,3,2-dioxaborolane (5.6 g, 21.4 mmol) and K₂CO₃ (6.3 g, 45.9 mmol) in 1,4-dioxane (150 mL) and H₂O (30 mL) under an atmosphere of N₂ was added Pd(dppf)Cl₂·DCM (1.3 g, 3.1 mmol). The mixture was heated to 80° C. and stirred for 4 h, then diluted with EtOAc (500 mL), washed with brine, dried over Na₂SO₄, and filtered. The filtrate was concentrated under reduced pressure and the residue was purified by silica gel column chromatography to give 3-(5-chloro-2-(2-(2-methoxyethyl)phenyl)-1H-pyrrolo[3,2-b]pyridin-3-yl)-2-methylpropanoate (5.5 g, 50% yield) as a viscous oil. LCMS (ESI): m/z: [M+H] calc'd for C₂₂H₂₅ClN₂O₃ 400.2; found 401.2.

Step 5. A mixture of ethyl 3-(5-chloro-2-(2-(2-methoxyethyl)phenyl)-1H-pyrrolo[3,2-b]pyridin-3-yl)-2-methylpropanoate (5.5 g, 13.8 mmol), Cs₂CO₃ (8.9 g, 27.5 mmol), and EtI (3.5 g, 27.5 mmol) in DMF (30 mL) at rt was stirred for 10 h. The mixture was diluted with EtOAc (100 mL), washed with brine (20 mL×4), dried over Na₂SO₄, filtered, and concentrated in vacuo. The filtrate was concentrated under reduced pressure and the residue was purified by silica gel column chromatography to give ethyl 3-(5-chloro-1-ethyl-2-(2-(2-methoxyethyl)phenyl)-1H-pyrrolo[3,2-b]pyridin-3-yl)-2-methylpropanoate (5.6 g, 95% yield) as a viscous oil. LCMS (ESI): m/z: [M+H] calc'd for C₂₅H₃₁ClN₂O₃ 428.2; found 429.2.

Step 6. To a mixture of ethyl 3-(5-chloro-1-ethyl-2-(2-(2-methoxyethyl)phenyl)-1H-pyrrolo[3,2-b]pyridin-3-yl)-2-methylpropanoate (5.4 g, 12.6 mmol) in THF (50 mL) at −65° C. was added 2M LDA (25 mL, 50 mmol) and stirred at −65° C. for 1 h. Mel (3.6 g, 25 mmol) was added and the mixture was stirred at −65° C. for 2.5 h, then aqueous NH₄Cl and EtOAc (50 mL) were added. The aqueous and organic layers were separated and the organic layer was washed with brine (30 mL), dried over Na₂SO₄, and filtered. The filtrate was concentrated under reduced pressure and the residue was purified by silica gel column chromatography to give ethyl 3-(5-chloro-1-ethyl-2-(2-(2-methoxyethyl)phenyl)-1H/pyrrolo[3,2-b]pyridin-3-yl)-2,2-dimethylpropanoate (3.2 g, 57% yield) as a viscous oil. LCMS (ESI): m/z: [M+H] calc'd for C₂₅H₃₁ClN₂O₃ 442.2; found 443.2.

Step 7. To a mixture of ethyl 3-(5-chloro-1-ethyl-2-(2-(2-methoxyethyl)phenyl)-1H-pyrrolo[3,2-b]pyridin-3-yl)-2,2-dimethylpropanoate (1.0 g, 2.3 mmol) in THF (10 mL) at 5° C. was added LiBH₄ (196 mg, 9.0 mmol). The mixture was heated to 65° C. and stirred for 2 h then aqueous NH₄Cl and EtOAc (50 mL) added. The aqueous and organic layers were separated and the organic layer was washed with brine (30 mL), dried over Na₂SO₄, and filtered. The filtrate was concentrated under reduced pressure and the residue was purified by silica gel column chromatography to give 3-(5-chloro-1-ethyl-2-(2-(2-methoxyethyl)phenyl)-1H-pyrrolo[3,2-b]pyridin-3-yl)-2,2-dimethylpropan-1-ol (0.75 g, 82% yield) as a solid. LCMS (ESI): m/z: [M+H] calc'd for C₂₃H₂₉ClN₂O₂ 400.2; found 401.2.

### Intermediate 11: Methyl (3S)-1-{(2S)-2-(tert-butoxycarbonyl)amino-3-[3-fluoro-5-(4,4,5,5-tetramethyl-1,3,2-dioxaborolan-2-yl)phenyl]propanoyl}-1,2-diazinane-3-carboxylate

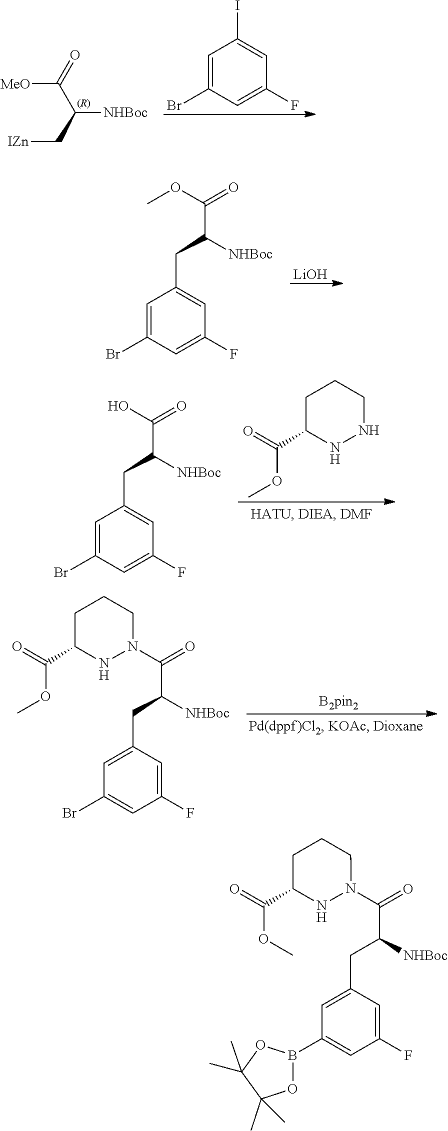

Step 1. To a stirred solution of methyl (2R)-2-{[(tert-butoxy)carbonyl]amino}-3-(iodozincio)propanoate (12 g, 30 mmol, 1.2 eq) in DMF (100 mL) was added 1-bromo-3-fluoro-5-iodobenzene (7.5 g, 25 mmol, 1 eq) and Pd(PPh₃)₂Cl₂ (1.7 g, 2.5 mmol, 0.1 equiv) at 20° C. under N₂ atmosphere. The resulting mixture was stirred for 2 hrs at 65° C. under N₂ atmosphere. The reaction mixture was quenched with water and extracted with EA (200 mL×2). The organic phase was washed with water (200 mL×1) and brine (100 mL×1) and concentrated to dryness to give a residue. The residue was purified by prep-TLC (PE/EA=10/1) to afford methyl 3-(3-bromo-5-fluorophenyl)-2-{[(tert-butoxy)carbonyl]amino}propanoate (6 g, 58% yield) as a colorless oil. LCMS (ESI) m/z=398.1 [M+Na], calculated for C₁₅H₁₉BrFNO₄: 375.0.

Step 2. To a solution of methyl 3-(3-bromo-5-fluorophenyl)-2-{[(tert-butoxy)carbonyl]amino}propanoate (3.2 g, 8.5 mmol, 1 eq) in THF (50 mL) was added Lithium hydroxide (610.7 mg, 25.5 mmol, 3 eq) in H₂O (10 mL). Then the reaction mixture was stirred at 20° C. for 1 h. The mixture was adjusted to pH=5.0 with 1 M HCl aqueous solution. The mixture was quenched with H₂O (150 mL) and extracted with EA (200 mL×3). The combined organic layers was washed bine (50 mL), dried over Na₂SO₄ and concentrated to afford 3-(3-bromo-5-fluorophenyl)-2-{[(tert-butoxy)carbonyl]amino}propanoic acid (2.65 g, 68% yield) as a white solid. LCMS (ESI) m/z=384.1 [M+Na]⁺, calculated for C₁₄H₁₅BrFNO₄ MW: 361.0.

Step 3. To a mixture of 3-(3-bromo-5-fluorophenyl)-2-{[(tert-butoxy)carbonyl]amino}propanoic acid (2.3 g, 6.4 mmol, 1 eq) and methyl (3S)-1,2-diazinane-3-carboxylate (1.66 g, 11.5 mmol, 1.8 eq) in DMF (150 mL) was added HATU (4.9 g, 12.8 mmol, 2 eq) and DIEA (16.5 g, 128 mmol, 20 eq) in DMF (50 mL) at 0° C. Then the reaction mixture was stirred at 0° C. for 1 h. The mixture was quenched with H₂O (100 mL) and extracted with EA (300 mL×3). The combined organic layers was washed bine (50 mL), dried over Na₂SO₄ and concentrated to give the residue, which was purified by Pre-HPLC eluting with acetonitrile in water (0.1% FA) from 60% to 70% in 10 minutes to give methyl (3S)-1-[(2S)-3-(3-bromo-5-fluorophenyl)-2-{[(tert-butoxy)carbonyl]amino}propanoyl]-1,2-diazinane-3-carboxylate (2.7 g, 78% yield) as a pale yellow solid. LCMS (ESI) m/z=510.1 [M+Na]⁺, calculated for C₂₀H₂₇BrFNO₅: 487.1.

Step 4. A mixture of methyl (S)-1-((S)-3-(3-bromo-5-fluorophenyl)-2-((tert-butoxycarbonyl)amino)propanoyl)hexahydropyridazine-3-carboxylate (3 g, 6.16 mmol, 1 eq), 4,4,5,5-tetramethyl-2-(4,4,5,5-tetramethyl-1,3,2-dioxaborolan-2-yl)-1,3,2-dioxaborolane (1.9 g, 7.4 mmol, 1.2 eq), KOAc (900 mg, 9.24 mmol, 1.5 eq) and Pd(dppf)Cl₂DCM (0.3 g, 0.37 mmol, 0.05 eq) in dioxane (50 mL) was heated at 100° C. for 17 h under N₂ atmosphere. The mixture was concentrated and purified by column chromatography (DCM/MeOH=100/1 to 40/1) to give methyl (3S)-1-(2S)-2-{(tert-butoxycarbonyl)amino-3-[3-fluoro-5-(4,4,5,5-tetramethyl-1,3,2-dioxaborolan-2-yl)phenyl]propanoyl}-1,2-diazinane-3-carboxylate (2.6 g, 79% yield) as a yellow oil. LCMS (ESI) m/z=536.2 [M+H]⁺, calculated for C₂₆H₃₉BFNO₇: 535.3.

Compounds A341 and A342 may be prepared using methods disclosed herein via Intermediate 11.

### Example A75. Synthesis of two atropisomers of (2S)—N-[(8S,14S,20M)-22-ethyl-4-hydroxy-21-{2-[(1S)-1-methoxyethyl]pyridin-3-yl}-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide

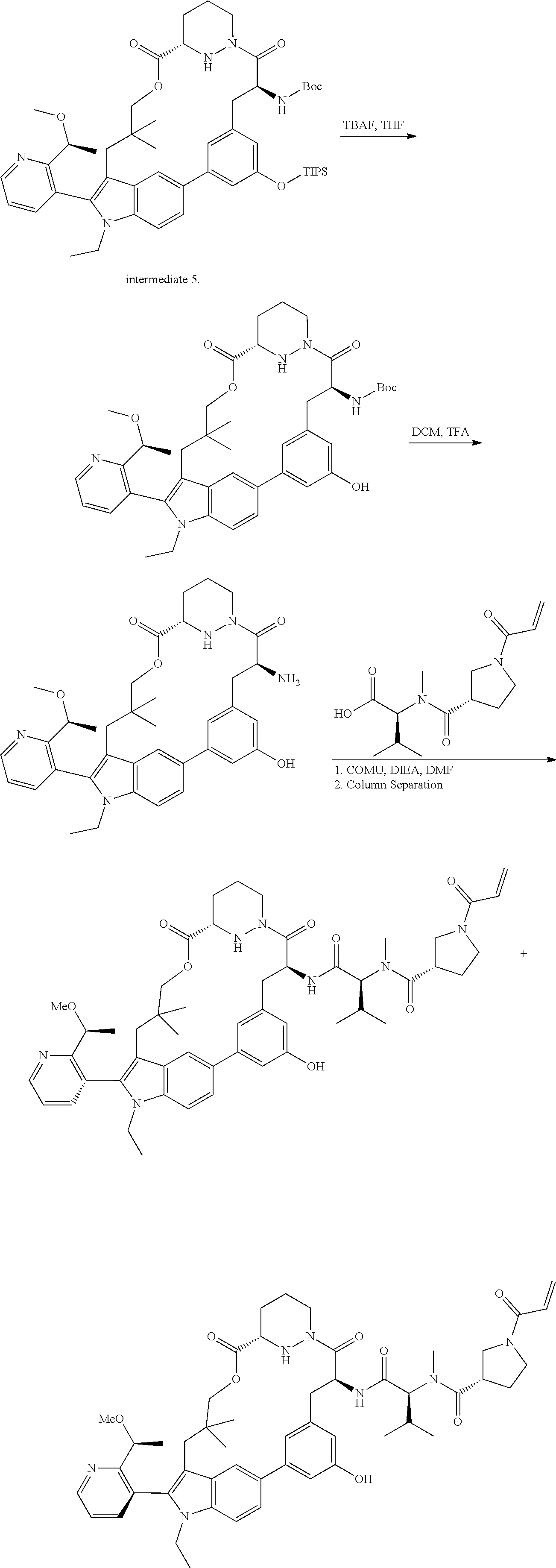

Step 1. To a stirred mixture of tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (18.0 g, 20.1 mmol) in THF (180 mL) at 0° C. was added a 1M solution of TBAF in THF (24.1 mL, 24.1 mmol). The mixture was stirred at 0° C. for 1 h, then diluted with brine (1.5 L) and extracted with EtOAc (3×1 L). The combined organic layers were washed with brine (2×500 mL), dried over anhydrous Na₂SO₄, and filtered. The filtrate was concentrated under reduced pressure and the residue was purified by silica gel column chromatography to give tert-butyl ((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (11.5 g, 69% yield) as a solid. LCMS (ESI): m/z: [M+H] calc'd for C₄₂H₅₃N₅O₇ 739.4; found 740.4.

Step 2. To a stirred mixture of tert-butyl ((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (11.5 g, 15.5 mmol) in DCM (120 mL) at 0° C. was added TFA (60 mL, 808 mmol). The mixture was stirred at 0° C. for 1 h, then concentrated under reduced pressure and the residue again concentrated under reduced pressure with toluene (20 mL; repeated ×3) to give (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (12 g, crude), which was used directly in the next step without further purification. LCMS (ESI): m/z: [M+H] calc'd for C₃₇H₄₅N₅O₅ 639.3; found 640.6.

Step 3. To a stirred mixture of (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (11.9 g, 18.6 mmol) in DMF (240 mL) at 0° C. under an atmosphere of N₂ was added DIPEA (48.1 g, 372 mmol), (2S)-3-methyl-2-[N-methyl-1-[(3S)-1-(prop-2-enoyl)pyrrolidin-3-yl]formamido]butanoic acid (9.45 g, 33.5 mmol) and COMU (11.95 g, 27.9 mmol) in portions. The mixture was stirred ay 0° C. for 90 min, then diluted with brine (1.5 L) and extracted with EtOAc (3×1 L). The combined organic layers were washed with brine (2×500 mL), dried over anhydrous Na₂SO₄, and filtered. After filtration, the filtrate was concentrated under reduced pressure. The filtrate was concentrated under reduced pressure and the residue was purified by silica gel column chromatography (×2) to give two atropisomers of (2S)—N-[(8S,14S,20M)-22-ethyl-4-hydroxy-21-{2-[(1S)-1-methoxyethyl]pyridin-3-yl}-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide (2.7 g, 15.5%, yield) and (4.2 g, 24.7% yield) both as solids. LCMS (ESI): m/z: [M+H] calc'd for C₅₁H₆₅N₇O₈ 903.5; found 904.7; ¹H NMR (400 MHz, DMSO-d₆) δ 9.35-9.27 (m, 1H), 8.77 (dd, J=4.7, 1.7 Hz, 1H), 7.95 (dq, J=6.2, 2.0 Hz, 2H), 7.55 (ddd, J=28.0, 8.2, 4.3 Hz, 3H), 7.08 (dd, J=37.9, 6.2 Hz, 2H), 6.69-6.48 (m, 2H), 6.17 (ddt, J=16.7, 7.2, 2.3 Hz, 1H), 5.74-5.62 (m, 1H), 5.43-5.34 (m, 1H), 5.12-5.00 (m, 1H), 4.25 (d, J=12.3 Hz, 1H), 4.17-3.99 (m, 3H), 3.89-3.65 (m, 4H), 3.66-3.45 (m, 3H), 3.12 (s, 4H), 2.95-2.70 (m, 6H), 2.41-2.06 (m, 5H), 1.99-1.88 (m, 1H), 1.82 (d, J=12.1 Hz, 2H), 1.54 (t, J=12.0 Hz, 1H), 1.21 (dd, J=6.3, 2.5 Hz, 3H), 1.11 (t, J=7.1 Hz, 3H), 0.99-0.88 (m, 6H), 0.79 (ddd, J=27.8, 6.7, 2.1 Hz, 3H), 0.48 (d, J=3.7 Hz, 3H) and LCMS (ESI): m/z: [M+H] calc'd for C₅₁H₆₅N₇O₈ 903.5; found 904.7; ¹H NMR (400 MHz, DMSO-d₆) δ 9.34-9.27 (m, 1H), 8.77 (dd, J=4.7, 1.7 Hz, 1H), 8.17-7.77 (m, 3H), 7.64-7.43 (m, 3H), 7.33 (d, J=13.7 Hz, 1H), 7.05-6.94 (m, 1H), 6.69-6.41 (m, 2H), 6.26-5.94 (m, 1H), 5.73-5.63 (m, 1H), 5.50-5.20 (m, 2H), 4.40-4.15 (m, 3H), 4.00-3.40 (m, 9H), 3.11 (d, J=4.4 Hz, 3H), 2.93-2.60 (m, 8H), 2.29-2.01 (m, 3H), 1.99 (s, 1H), 1.87-1.75 (m, 2H), 1.73-1.47 (m, 2H), 1.40 (d, J=6.0 Hz, 3H), 1.01-0.88 (m, 6H), 0.85-0.65 (m, 7H), 0.56 (s, 3H).

### Example A89. Synthesis of (2S)—N-[(8S,14S)-22-ethyl-4-hydroxy-18,18-dimethyl-21-[6-(4-methylpiperazin-1-yl)pyridin-3-yl]-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide

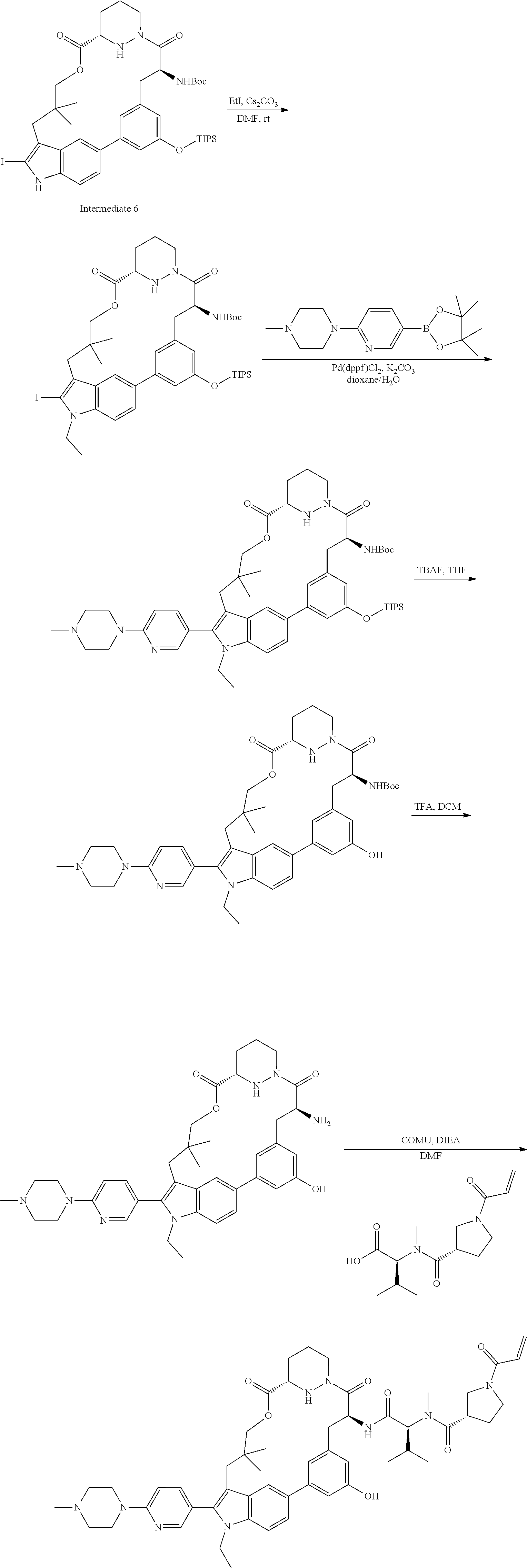

Step 1. To a mixture of tert-butyl ((6³S,4S)-1²-iodo-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (240.00 mg, 0.279 mmol, 1.00 equiv) and Cs₂CO₃ (182 mg, 0.558 mmol, 2 equiv) in DMF (5.00 mL) was added ethyl iodide (113.45 mg, 0.727 mmol, 2.60 equiv) dropwise at 0° C. The reaction was stirred for 16 h at 25° C. The resulting mixture was diluted with water (10 mL) and extracted with EtOAc (3×10 mL). The combined organic layers were washed with brine (3×10 mL), and dried over anhydrous Na₂SO₄. The filtrate was concentrated under reduced pressure and the remaining residue was purified by silica gel column chromatography to afford tert-butyl ((6³S,4S)-1¹-ethyl-1²-iodo-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (190 mg, 77% yield) as a yellow solid.

Step 2. A mixture of tert-butyl ((6³S,4S)-1¹-ethyl-1²-iodo-10,10-dimethyl-5,7-dioxo-2⁵-((triisopropylsilyl)oxy)-6¹,6²,6³,6⁴,6⁵,6⁶-hexahydro-1¹H8-oxa-1(5,3)-indola-6(1,3)-pyridazina-2(1,3)-benzenacycloundecaphane-4-yl)carbamate (500 mg, 0.54 mmol), 1-methyl-4-[5-(4,4,5,5-tetramethyl-1,3,2-dioxaborolan-2-yl)pyridin-2-yl]piperazine (257 mg, 0.8 mmol), Pd(dppf)Cl₂ (83 mg, 0.11 mmol) and K₂COS (156 mg, 1.1 mmol) in 1,4-dioxane (25 mL) and H₂O (5 mL) under an atmosphere of Ar was stirred at 80° C. for 2 h. The mixture was concentrated under reduced pressure and the residue was purified by prep-TLC to afford tert-butyl ((6³S,4S)-1¹-ethyl-10,10-dimethyl-1²-(6-(4-methylpiperazin-1-yl)pyridin-3-yl)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (400 mg, 76% yield) as a solid. LCMS (ESI): m/z [M+H] calc'd for C₅₃H₇₇N₇O₆Si 935.6; found 936.6.

Step 3. A mixture of tert-butyl ((6³S,4S)-1¹-ethyl-10,10-dimethyl-1²-(6-(4-methylpiperazin-1-yl)pyridin-3-yl)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 0.36 mmol) and 1M TBAF in THF (0.4 mL, 0.4 mmol) in THF (5 mL) was stirred at 0° C. for 1 h. The mixture was concentrated under reduced pressure and the residue was purified by silica gel column chromatography to give tert-butyl ((6³S,4S)-1¹-ethyl-2⁵-hydroxy-10,10-dimethyl-1²-(6-(4-methylpiperazin-1-yl)pyridin-3-yl)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (290 mg, 100% yield) as a solid. LCMS (ESI): m/z: [M+H] calc'd for C₄₄H₅₇N₇O₆ 779.4; found 780.4.

Step 4. A mixture of tert-butyl ((6³S,4S)-1¹-ethyl-2⁵-hydroxy-10,10-dimethyl-1²-(6-(4-methylpiperazin-1-yl)pyridin-3-yl)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (300 mg, 0.37 mmol) in TFA (5 mL) and DCM (5 mL) was stirred at rt for 1 h. The mixture was concentrated under reduced pressure to give (6³S,4S)-4-amino-1¹-ethyl-2⁵-hydroxy-10,10-dimethyl-1²-(6-(4-methylpiperazin-1-yl)pyridin-3-yl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300 mg, crude) as a solid. LCMS (ESI): m/z: [M+H] calc'd for C₃₉H₄₉N₇O₄ 679.4; found 680.3.

Step 5. To a mixture of (6³S,4S)-4-amino-1¹-ethyl-2⁵-hydroxy-10,10-dimethyl-1²-(6-(4-methylpiperazin-1-yl)pyridin-3-yl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300 mg, 0.36 mmol) in DMF (3 mL) at 0° C. under an atmosphere of N₂ was added DIPEA (0.96 mL, 5.4 mmol) and (2S)-3-methyl-2-[N-methyl-1-[(3S)-1-(prop-2-enoyl)pyrrolidin-3-yl]formamido]butanoic acid (213 mg, 0.72 mmol), followed by dropwise addition of COMU (243 mg, 0.56 mmol). H₂O was added at 0° C. and the mixture was extracted with EtOAc (3×10 mL). The combined organic layers were washed with brine (3×10 mL), dried over anhydrous Na₂SO₄, and filtered. The filtrate was concentrated under reduced pressure and the crude residue was purified by Prep-HPLC to give (2S)—N-[(8S,14S)-22-ethyl-4-hydroxy-18,18-dimethyl-21-[6-(4-methylpiperazin-1-yl)pyridin-3-yl]-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide (45 mg, 13.2% yield) as a solid. LCMS (ESI): m/z: [M+H] calc'd for C₅₃H₆₉N₉O₇ 943.5, found 944.8; ¹H NMR (400 MHz, DMSO-d₆) δ 9.39-9.23 (m, 1H), 8.64-8.60 (m, 1H), 8.19-8.16 (m, 1H), 8.15 (d, J=6.2 Hz, 1H). 7.86 (s, 1H). 7.66-7.62 (m, 1H), 7.56-7.54 (m, 1H), 7.50-7.43 (m, 1H). 7.13-7.11 (m, 1H), 7.03-6.95 (m, 1H). 6.70-6.47 (m, 2H), 6.17 (ddt, J=16.8, 6.4, 2.8 Hz, 1H). 5.76-5.63 (m, 1H), 5.45-5.33 (m, 1H), 5.11 (m, 1H), 4.75-4.72 (m, 1H), 4.28-4.24 (m, 1H), 4.11-3.98 (m, 4H), 3.91-3.76 (m, 1H), 3.73-3.71 (m, 1H), 3.59-3.56 (m, 7H), 3.51-3.40 (m, 2H), 3.08-2.94 (m, 1H). 2.94-2.92 (m, 2H), 2.92-2.87 (m, 2H), 2.86-2.83 (m, 2H), 2.80-2.65 (m, 2H), 2.83-2.82 (m, 3H), 2.28-2.25 (m, 3H), 2.08-2.05 (m, 2H), 2.02-1.96 (m, 1H). 1.87-1.78 (m, 1H), 1.74-1.66 (m, 1H), 1.56-1.48 (m, 1H), 1.11-1.08 (m, 4H), 0.99-0.92 (m, 2H), 0.89-0.87 (m, 5H), 0.82-0.73 (m, 2H).

### Example A115. Synthesis of two atropisomers of (2S)—N-[(8S,14S,20P)-22-ethyl-21-{4-[(1S)-1-methoxyethyl]pyridin-3-yl}-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide

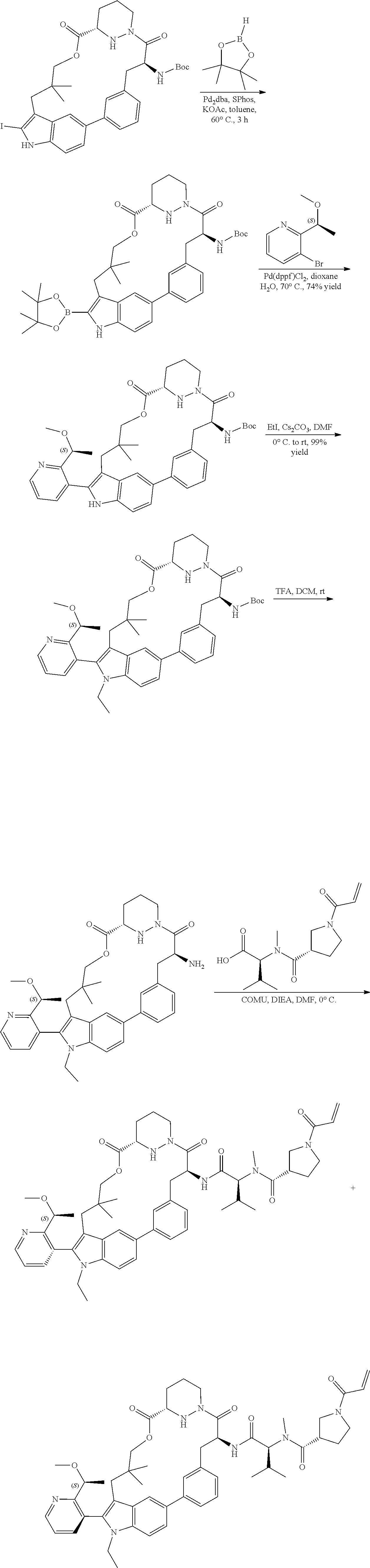

Step 1. A 1 L round-bottom flask was charged with tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22.00 g, 32.042 mmol, 1.00 equiv), toluene (300.00 mL), Pd₂(dba)₃ (3.52 g, 3.845 mmol, 0.12 equiv), S-Phos (3.95 g, 9.613 mmol, 0.30 equiv), and KOAc (9.43 g, 96.127 mmol, 3.00 equiv) at room temperature. To the mixture was added 4,4,5,5-tetramethyl-1,3,2-dioxaborolane (26.66 g, 208.275 mmol, 6.50 equiv) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford tert-butyl ((6³S,4S)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22 g, 90%) as a light yellow solid. ESI-MS m/z=687.3 [M+H]⁺; Calculated MW: 686.4.

Step 2. A mixture of tert-butyl ((6³S,4S)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.0 g, 2.8 mmol), 3-bromo-2-[(1S)-1-methoxyethyl]pyridine (0.60 g, 2.8 mmol), Pd(dppf)Cl₂ (0.39 g, 0.5 mmol), and K₃PO₄ (1.2 g, 6.0 mmol) in 1,4-dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to give tert-butyl ((6³S,4S)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.5 g, 74% yield) as a solid. LCMS (ESI): m/z: [M+H] calc'd for C₄₀H₄₉N₅O₆ 695.4; found 696.5.

Step 3. A mixture of tert-butyl ((6³S,4S)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.5 g, 2.1 mmol), Cs₂CO₃ (2.1 g, 6.3 mmol), and ethyl iodide (0.43 mL, 5.1 mmol) in DMF (50 mL) was stirred at 0° C. for 16 h. The mixture was quenched at 0° C. with H₂O and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.5 g, 99% yield) as a solid. LCMS (ESI): m/z: [M+H] calc'd for C₄₂H₅₃N₅O₆ 723.4; found 724.6.

Step 4. A mixture of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.3 g, 1.7 mmol) in TFA (10 mL) and DCM (20 mL) was stirred at 0° C. for 2 h. The mixture was concentrated under reduced pressure to afford (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.30 g, crude) as a solid. LCMS (ESI): m/z: [M+H] calc'd for C₃₇H₄₅N₅O₄ 623.3; found 624.4.

Step 5. Into a 40-mL vial purged and maintained with an inert atmosphere of Ar, was placed (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (250 mg, 0.4 mmol), (2S)-3-methyl-2-[N-methyl-1-[(3S)-1-(prop-2-enoyl)pyrrolidin-3-yl]formamido]butanoic acid (226 mg, 0.8 mmol), DIPEA (774 mg, 6.0 mmol), and DMF (3 mL). A solution of COMU (257 mg, 0.6 mmol) in DMF (2 mL) was added at 0° C. and the resulting mixture was stirred at 0° C. for 1 h. The mixture was filtered, the filtrate was concentrated under reduced pressure, and the residue was purified by prep-HPLC to give two atropisomers of (2S)—N-[(8S,14S,20P)-22-ethyl-21-{4-[(1S)-1-methoxyethyl]pyridin-3-yl}-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide (56 mg, 15% yield) and (46 mg, 13% yield) both as a solid. LCMS (ESI): m/z [M+H] calc'd for C₅₁H₆₅N₇O₇ 887.5; found 888.4; ¹H NMR (400 MHz, DMSO-d₆) δ 8.81 (s, 1H), 8.07 (s, 1H), 8.05-7.96 (m, 1H), 7.78-7.45 (m, 5H), 7.41-7.08 (m, 2H), 6.66-6.58 (m, 1H), 6.18 (d, J=17.0 Hz, 1H), 5.75-5.67 (m, 1H), 5.46-5.31 (m, 1H), 5.16-5.04 (m, 1H), 4.75 (dd, J=10.9, 4.5 Hz, 1H), 4.31-4.21 (m, 2H), 4.11-3.95 (m, 3H), 3.87-3.71 (m, 5H), 3.74-3.54 (m, 3H), 3.11 (s, 4H), 2.95 (d, J=9.7 Hz, 2H), 2.85-2.72 (m, 3H), 2.31-2.04 (m, 3H), 1.88-1.47 (m, 2H), 1.24-1.21 (m, 3H), 1.16-1.08 (m, 3H), 1.03-0.91 (m, 6H), 0.85-0.74 (m, 3H), 0.51-0.46 (m, 3H) and LCMS (ESI): m/z: [M+H] calc'd for C₅₁H₆₅N₇O₇ 887.5; found 888.4; ¹H NMR (400 MHz, DMSO-d₆) δ 8.77 (s, 1H), 8.71-8.63 (m, 0.5H), 8.23-8.17 (m, 0.5H), 8.00 (s, 1H), 7.85 (t, J=9.9 Hz, 2H), 7.77-7.62 (m, 3H), 7.57-7.50 (m, 1H), 7.33-7.22 (m, 1H), 7.15-7.06 (m, 1H), 6.73-6.56 (m, 1H), 6.17 (ddd, J=16.7, 6.1, 2.7 Hz, 1H), 5.76-5.64 (m, 1H), 5.49-5.29 (m, 2H), 4.70 (dd, J=10.8, 3.5 Hz, 1H), 4.33-4.22 (m, 3H), 4.14-3.95 (m, 2H), 3.86-3.77 (m, 1H), 3.72-3.65 (m, 2H), 3.61 (t, J=10.6 Hz, 3H), 3.46-3.42 (m, 1H), 3.13 (d, J=4.8 Hz, 3H), 2.99 (d, J=14.4 Hz, 1H), 2.95-2.70 (m, 6H), 2.24-1.99 (m, 4H), 1.95-1.44 (m, 4H), 1.40 (d, J=6.1 Hz, 3H), 0.98-0.87 (m, 6H), 0.86-0.64 (m, 6H), 0.64-0.54 (m, 3H).

### Example A2. Synthesis of (2S)—N-[(8S,14S)-4-amino-22-ethyl-21-[2-(2-methoxyethyl)phenyl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide

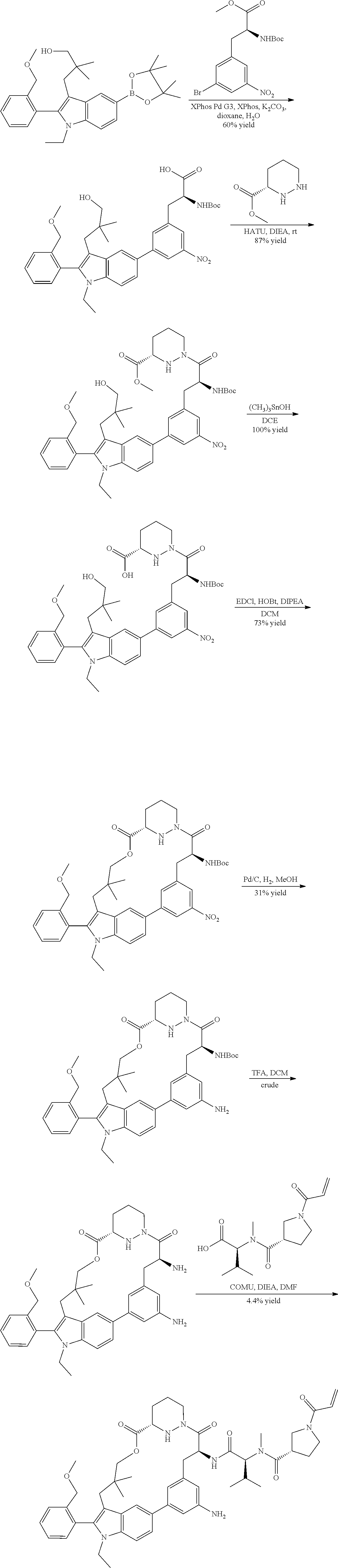

Step 1. Into a 25 mL sealed tube were added 3-[1-ethyl-2-[2-(methoxymethyl)phenyl]-5-(4,4,5,5-tetramethyl-1,3,2-dioxaborolan-2-yl)indol-3-yl]-2,2-dimethylpropan-1-ol (590 mg, 1.2 mmol), methyl (2S)-3-(3-bromo-5-nitrophenyl)-2-[(tert-butoxycarbonyl)amino]propanoate (747 mg, 1.9 mmol), XPhos Pd G3 (105 mg, 0.12 mmol), XPhos (71 mg, 0.15 mmol), K₂CO₃ (427 mg, 3.1 mmol), and 1,4-dioxane (2 mL) under an atmosphere of N₂ at rt. The mixture was heated to 60° C. and stirred overnight, then cooled and H₂O added. The mixture was extracted with EtOAc (3×20 mL) and the combined organic layers were washed with brine (1×20 mL), dried over anhydrous Na₂SO₄, and filtered. The filtrate was concentrated under reduced pressure and the residue was purified by silica gel column chromatography to give (2S)-2-[(tert-butoxycarbonyl)amino]-3-[3-[1-ethyl-3-(3-hydroxy-2,2-dimethylpropyl)-2-[2-(methoxymethyl)phenyl]indol-5-yl]-5-nitrophenyl]propanoic acid (500 mg, 61% yield) as a solid. LCMS (ESI): m/z [M+H] calc'd for C₃₇H₄₅N₃O₈ 659.3; found 660.4.

Step 2. A mixture of (2S)-2-[(tert-butoxycarbonyl)amino]-3-[3-[1-ethyl-3-(3-hydroxy-2,2-dimethylpropyl)-2-[2-(methoxymethyl)phenyl]indol-5-yl]-5-nitrophenyl]propanoic acid (500 mg, 0.79 mmol), methyl (3S)-1,2-diazinane-3-carboxylate (164 mg, 1.1 mmol), DCM (6 mL), DIPEA (294 mg, 2.3 mmol) and HATU (432 mg, 1.1 mmol) was stirred at 0° C. for 1 h under an atmosphere of air. H₂O was added and the mixture was extracted with DCM (3×20 mL), then the combined organic layers were dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl)amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylate (520 mg, 87% yield) as a solid. LCMS (ESI): m/z: [M+H] calc'd for C₄₃H₅₅N₅O₉ 785.4; found 786.8.

Step 3. Into a 40 mL sealed tube were added methyl (3S)-1-[(2S)-2-[(tert-butoxycarbonyl)amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylate (510 mg, 0.65 mmol), DCE (5 mL) and trimethyltin hydroxide (587 mg, 3.3 mmol) at rt under an atmosphere of air. The mixture was heated to 60° C. and stirred overnight, cooled, and diluted with DCM (20 mL). The mixture was washed with 0.1 N KHSO₄ (3×20 mL), dried over anhydrous Na₂SO₄, filtered, and the filtrate was concentrated under reduced pressure to give (3S)-1-[(2S)-2-[(tert-butoxycarbonyl)amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylic acid (500 mg, 100%) as a solid. LCMS (ESI): m/z: [M+H] calc'd for C₄₂H₅₃N₅O₉ 771.4; found 772.7.

Step 4. A mixture of (3S)-1-[(2S)-2-[(tert-butoxycarbonyl)amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylic acid (490 mg, 0.64 mmol), DCM (100 mL), DIPEA (2.5 g, 19.0 mmol), HOBT (429 mg, 3.2 mmol), and EDCI (3.65 g, 19.0 mmol) at room temperature was stirred at rt overnight under an atmosphere of air. H₂O was added and the mixture was extracted with DCM (3×60 mL), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-(methoxymethyl)phenyl)-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 73% yield) as a solid. LCMS (ESI): m/z: [M+H] calc'd for C₄₂H₅₁N₅O₈ 753.4; found 754.2.

Step 5. A mixture of tert-butyl ((6³S,4S)-1¹-ethyl-1²-(2-(methoxymethyl)phenyl)-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate (200 mg, 0.27 mmol), MeOH (4 mL), and Pd on carbon (20 mg) was stirred at rt for 2 h under an atmosphere of H₂. The mixture was filtered, the filter cake was washed with MeOH (3×5 mL), and the filtrate was concentrated under reduced pressure to give tert-butyl ((6³S,4S)-2⁵-amino-1¹-ethyl-1²-(2-(methoxymethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (60 mg, 31% yield) as a solid. LCMS (ESI): m/z: [M+H] calc'd for C₄₂H₅₃N₅O₆ 723.4; found 724.4.

Step 6. Into an 8 mL vial were added tert-butyl ((6³S,4S)-2⁵-amino-1¹-ethyl-1²-(2-(methoxymethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (50 mg, 0.07 mmol), DCM (1 mL), and TFA (158 mg, 1.4 mmol) at 0° C. under an atmosphere of air. The mixture was stirred for at 0° C. for 2 h then concentrated under reduced pressure to give (6³S,4S)-2⁵,4-diamino-1¹-ethyl-1²-(2-(methoxymethyl)phenyl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (45 mg) as a solid, which was used directly in the next step directly without further purification. LCMS (ESI): m/z: [M+H] calc'd for C₃₇H₄₅N₅O₄ 623.3; found 624.4.

Step 7. Into an 8 mL vial were added (6³S,4S)-2⁵,4-diamino-1¹-ethyl-1²-(2-(methoxymethyl)phenyl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (40 mg, 0.06 mmol), DMF (1 mL), DIPEA (75 mg, 0.58 mmol), and COMU (41 mg, 0.1 mmol) at 0° C. under an atmosphere of air. The mixture was stirred at 0° C. for 1 h, then H₂O added. The mixture was extracted with EtOAc (3×30 mL), the combined organic layers were concentrated under reduced pressure, and purified by prep-HPLC to give (2S)—N-[(8S,14S)-4-amino-22-ethyl-21-[2-(2-methoxyethyl)phenyl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide (2.5 mg, 4.4% yield) as a solid. LCMS (ESI): m/z: [M+H] calc'd for C₅₁H₆₅N₇O₇ 887.5; found 888.6; ¹H NMR (400 MHz, DMSO-d₆) δ 8.74-8.55 (m, 1H), 7.89 (d, J=9.6 Hz, 1H), 7.66-7.53 (m, 1H), 7.57-7.47 (m, 6H), 7.32 (t, J=6.4 Hz, 1H), 6.85 (d, J=8.4 Hz, 2H), 6.70-6.55 (m, 1H), 6.24-6.12 (m, 1H), 5.69 (ddd, J=14.8, 8.0, 3.9 Hz, 1H), 5.41 (s, 1H), 5.09-4.80 (m, 2H), 4.26 (d, J=10.1 Hz, 2H), 4.19 (s, 2H), 4.17-4.06 (m, 1H), 4.02 (dd. J=12.0, 3.9 Hz, 1H), 3.92 (d, J=8.0 Hz, 3H), 3.78 (d, J=8.7 Hz, 5H), 3.29 (s, 2H), 3.14 (d, J=1.9 Hz, 1H), 2.98-2.92 (m, 1H), 2.87-2.68 (m, 3H), 2.62 (d, J=12.5 Hz, 3H), 2.15-1.99 (m, 4H), 1.80 (s, 1H), 1.68-1.53 (m, 2H), 1.08 (t, J=7.1 Hz, 1H), 0.98-0.88 (m, 6H), 0.82 (dd, J=23.3, 16.4 Hz, 3H), 0.74 (t, J=7.2 Hz, 3H), 0.44 (s, 2H), 0.43 (s, 3H).

### Example A118. Synthesis of (2S)—N-[(7S,13S)-21-ethyl-20-[2-(methoxymethyl)pyridin-3-yl]-17,17-dimethyl-8,14-dioxo-15-oxa-3-thia-9,21,27,28-tetraazapentacyclo[17.5.2.1^{2,5}.1^{9,13}.0^{22,26}]octacosa-1(25),2(25),4,19,22(26),23-hexaen-7-yl]-3-methyl-2-{N-methyl-1-[(3S)-1-(prop-2-enoyl)pyrrolidin-3-yl]formamido}butanamide

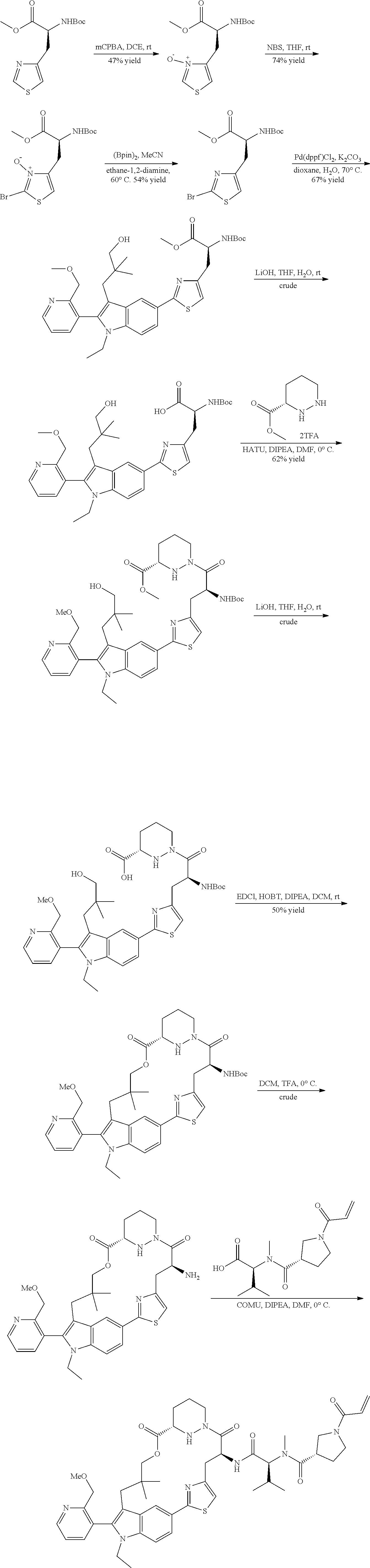

Step 1. A mixture of methyl (2S)-2-[(tert-butoxycarbonyl)amino]-3-(1,3-thiazol-4-yl)propanoate (2.08 g, 7.26 mmol) and mCPBA (1.88 g, 10.9 mmol) in DCE (15 mL) at 0° C. under an atmosphere of N was diluted with DCM (100 mL). The mixture was allowed to warm to rt and stirred for 16 h, then diluted with DCM, washed with H₂O (1×30 mL), dried over anhydrous Na₂SO₄, and filtered. The filtrate was concentrated under reduced pressure and the residue was purified by silica gel column chromatography to give 4-[(2S)-2-[(tert-butoxycarbonyl)amino]-3-methoxy-3-oxopropyl]-1,3-thiazol-3-ium-3-olate (1.15 g, 47% yield) as a solid. LCMS (ESI): m/z: [M+H] calc'd for C₁₂H₁₈N₂O₅S 302.1; found 303.2.

Step 2. To a mixture of 4-[(2S)-2-[(tert-butoxycarbonyl)amino]-3-methoxy-3-oxopropyl]-1,3-thiazol-3-ium-3-olate (1.15 g, 3.8 mmol) in THF at 0° C. under an atmosphere of N₂ was added NBS (0.74 g, 4.2 mmol) dropwise. The mixture was allowed to warm to rt and stirred for 2 h, then diluted with H₂O (500 mL) and extracted with EtOAc (3×500 mL). The combined organic layers were washed with water (2×30 mL), dried over anhydrous Na₂SO₄, and filtered. The filtrate was concentrated under reduced pressure and the residue was purified by silica gel column chromatography to give 2-bromo-4-[(2S)-2-[(tert-butoxycarbonyl)amino]-3-methoxy-3-oxopropyl]-1,3-thiazol-3-ium-3-olate (1.2 g, 74% yield) as a solid. LCMS (ESI): m/z: [M+H] calc'd for C₁₂H₁₇BrN₂O₅S 380.0; found 381.0.

Step 3. To a stirred mixture of 2-bromo-4-[(2S)-2-[(tert-butoxycarbonyl)amino]-3-methoxy-3-oxopropyl]-1,3-thiazol-3-ium-3-olate (1.2 g, 3.2 mmol) and 4,4,5,5-tetramethyl-2-(tetramethyl-1,3,2-dioxaborolan-2-yl)-1,3,2-dioxaborolane (1.04 g, 4.1 mmol) in MeCN at 70° C. under an atmosphere of N₂ was added ethane-1,2-diamine (1.89 g, 31.5 mmol) in portions. The mixture was cooled to 60° C. and the mixture was stirred overnight, then diluted with water (500 mL) and extracted with EtOAc (3×400 mL). The combined organic layers were washed with brine (1×50 mL), dried over anhydrous Na₂SO₄, and filtered. The filtrate was concentrated under reduced pressure and the residue was purified by silica gel column chromatography to give methyl (2S)-3-(2-bromo-1,3-thiazol-4-yl)-2-[(tert-butoxycarbonyl)amino]propanoate (653 mg, 54% yield) as a solid.

Step 4. A 50 mL sealed tube was charged with 3-[1-ethyl-2-[2-(methoxymethyl)pyridin-3-yl]-5-(4,4,5,5-tetramethyl-1,3,2-dioxaborolan-2-yl)indol-3-yl]-2,2-dimethylpropan-1-ol (1.00 g, 2.1 mmol), K₂CO₃ (727 mg, 5.2 mmol), Pd(dppf)Cl₂ (153 mg, 0.21 mmol), and 2,4-dibromo-1,3-thiazole (1.0 g, 4.2 mmol) at rt under an atmosphere of N₂, then 1,4-dioxane (1.0 mL) and H₂O (0.20 mL) were added. The mixture was heated to 70° C. and stirred for 4 h, then cooled, diluted with H₂O (100 mL) and extracted with EtOAc (3×100 mL). The combined organic layers were washed with brine (3×100 mL), dried over anhydrous Na₂SO₄, and filtered. The filtrate was concentrated under reduced pressure and the residue was purified by silica gel column chromatography to give 3-[5-(4-bromo-1,3-thiazol-2-yl)-1-ethyl-2-[2-(methoxymethyl)pyridin-3-yl]indol-3-yl]-2,2-dimethylpropan-1-ol (727 mg, 67% yield) as a solid. LCMS (ESI): m/z: [M+H] calc'd for C₃₄H₄₄N₄O₆S 636.3; found 637.3.

Step 5. To a stirred mixture of methyl (2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoate (636 mg, 1.0 mmol) and LiOH·H₂O (126 mg, 3.0 mmol) in THF at 0° C. under an atmosphere of N₂ was added H₂O (1.24 mL) portionwise. The mixture was allowed to warm to rt and stirred for 1 h, then diluted with water (300 mL) and extracted with EtOAc (3×300 mL). The combined organic layers were washed with brine (1×100 mL), dried over anhydrous Na₂SO₄, filtered, and the filtrate concentrated under reduced pressure to give (2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoic acid (622 mg, crude), which was used in the next step directly without further purification. LCMS (ESI): m/z: [M+H] calc'd for C₃₃H₄₂N₄O₆S 622.3; found 623.2.

Step 6. To a stirred mixture of (2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoic acid (622 mg, 1.0 mmol) and methyl (3S)-1,2-diazinane-3-carboxylate (288 mg, 2.0 mmol) in DMF at 0° C. under an atmosphere of N₂ was added HATU (570 mg, 1.5 mmol). The mixture was stirred at 0° C. for 1 h, then diluted with EtOAc and washed with H₂O (1×10 mL), dried over anhydrous Na₂SO₄, filtered, and the filtrate concentrated under reduced pressure to give methyl (3S)-1-[(2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylate (550 mg, 62% yield) as a solid. LCMS (ESI): m/z: [M+H] calc'd for C₃₉H₅₂N₆O₇S 748.4; found 749.6.

Step 7. (3S)-1-[(2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylic acid was synthesized in a manner similar to (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid except methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate was substituted with methyl (3S)-1-[(2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylate. LCMS (ESI): m/z: [M+H] calc'd for C₃₈H₅₀N₆O₇S 734.3; found 735.3.

Step 8. tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2(2,4)-thiazola-1(5,3)-indola-6(1,3)-pyridazinacycloundecaphane-4-yl)carbamate was synthesized in a manner similar to tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate except (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid was substituted with (3S)-1-[(2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylic acid. LCMS (ESI): m/z: [M+H] calc'd for C₃₈H₄₈N₆O₆S 716.3; found 717.4.

Step 9. To a stirred mixture of tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2(2,4)-thiazola-1(5,3)-indola-6(1,3)- pyridazinacycloundecaphane-4-yl)carbamate (253 mg) in DCM at 0° C. under an atmosphere of N₂ was added TFA (1.0 mL) dropwise. The mixture was stirred at 0° C. for 1 h, then concentrated under reduced pressure and then repeated using toluene (20 mL×3) to give (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2(2,4)-thiazola-1(5,3)-indola-6(1,3)-pyridazinacycloundecaphane-5,7-dione (253 mg, crude) as a solid. LCMS (ESI): m/z: [M+H] calc'd for C₃₃H₄₀N₆O₄S 616.3; found 617.3.

Step 10. (2S)—N-[(7S,13S)-21-ethyl-20-[2-(methoxymethyl)pyridin-3-yl]-17,17-dimethyl-8,14-dioxo-15-oxa-3-thia-9,21,27,28-tetraazapentacyclo[17.5.2.1^{2,5}.1^{9,13}.0^{22,26}]octacosa-1(25),2(25),4,19,22(26),23-hexaen-7-yl]-3-methyl-2-{N-methyl-1-[(3S)-1-(prop-2-enoyl)pyrrolidin-3-yl]formamido}butanamide was synthesized in a manner similar to (2S)—N-[(8S,14S)-4-amino-22-ethyl-21-[2-(2-methoxyethyl)phenyl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-(prop-2-enoyl)pyrrolidin-3-yl]formamido}butanamide except (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione was substituted with (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2(2,4)-thiazola-1(5,3)-indola-6(1,3)-pyridazinacycloundecaphane-5,7-dione. LCMS (ESI): m/z: [M+H] calc'd for C₄₇H₆₀N₈O₇S 880.4; found 881.6; ¹H NMR (400 MHz, DMSO-d₆) δ 8.75 (m, 1H), 8.55 (d, J=6.7 Hz, 1H), 8.32 (d, J=8.3 Hz, 1H), 7.99 (d, J=7.7 Hz, 1H), 7.65-7.51 (m, 3H), 7.11-6.92 (m, 1H), 6.72-6.56 (m, 1H), 6.18 (dd, J=16.8, 2.9 Hz, 1H), 5.82-5.65 (m, 1H), 5.61-5.46 (m, 1H), 5.02 (dd, J=24.2, 12.2 Hz, 1H), 4.69 (d, J=10.9 Hz, 1H), 4.37-4.11 (m, 5H), 4.05-3.79 (m, 4H), 3.76-3.50 (m, 6H), 3.47 (s, 2H), 3.08 (s, 3H), 3.04 (s, 1H), 2.98 (d, J=1.9 Hz, 1H), 2.95 (d, J=3.6 Hz, 2H), 2.83 (d, J=2.0 Hz, 2H), 2.24-2.03 (m, 4H), 1.81 (s, 2H), 1.56 (s, 1H), 1.11 (t, J=7.0 Hz, 2H), 1.02-0.87 (m, 8H), 0.80 (dd, J=24.6, 6.6 Hz, 3H), 0.41 (s, 2H), 0.31 (s, 1H).

### Example A194. Synthesis of (2S)—N-[(7S,13S)-21-ethyl-20-[2-(methoxymethyl)pyridin-3-yl]-17,17-dimethyl-8,14-dioxo-15-oxa-4-thia-9,21,27,28-tetraazapentacyclo[17.5.2.1^{2,5}.1^{9,13}.0^{22,26}]octacosa-1(25),2,5(25),19,22(26),23-hexaen-7-yl]-3-methyl-2-{N-methyl-1-[(3S)-1-(prop-2-enoyl)pyrrolidin-3-yl]formamido}butanamide

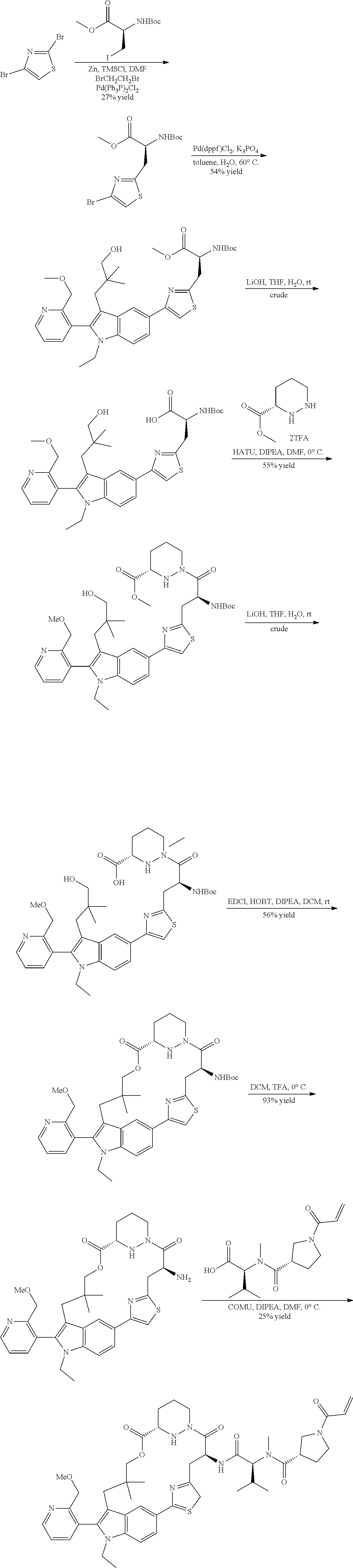

Step 1. A mixture of Zn (1.2 g, 182 mmol) and 1,2-dibromoethane (1.71 g, 9.1 mmol) and DMF (50 mL) was stirred for 30 min at 90° C. under an atmosphere of Ar. The mixture was allowed to rt, then TMSCI (198 mg, 1.8 mmol) was added dropwise over 30 min at rt. Methyl (2R)-2-[(tert-butoxycarbonyl) amino]-3-iodopropanoate (10.0 g, 30.4 mmol) in DMF (100 mL) was added dropwise over 10 min at rt. The mixture was heated to 35° C. and stirred for 2 h, then a mixture of 2,5-dibromo-1,3-thiazole (1.48 g, 60.8 mmol) and Pd(PPh₃)₂Cl₂ (2.1 g, 3.0 mmol) in DMF (100 mL) was added dropwise. The mixture was heated to 70° C. and stirred for 2 h, then filtered and the filtrate diluted with EtOAc (1 L) and washed with H₂O (3×1 L), dried with anhydrous Na₂SO₄, and filtered. The filtrate was concentrated under reduced pressure and the residue was purified by silica gel column chromatography to give methyl (2S)-3-(5-bromo-1,3-thiazol-2-yl)-2-[(tert-butoxycarbonyl)amino]propanoate (3 g, 27% yield) as a semi-solid. LCMS (ESI): m/z: [M+H] calc'd for C₁₂H₁₇BrN₂O₄S 364.0; found 365.1.

Step 2. Into a 20 mL sealed tube were added 3-[1-ethyl-2-[2-(methoxymethyl)pyridin-3-yl]-5-(4,4,5,5-tetramethyl-1,3,2-dioxaborolan-2-yl)indol-3-yl]-2,2-dimethylpropan-1-ol (100 mg, 0.21 mmol), K₃PO₄ (111 mg, 0.52 mmol), Pd(dppf)Cl₂ (15 mg, 0.02 mmol), methyl (2S)-3-(4-bromo-1,3-thiazol-2-yl)-2-[(tert-butoxycarbonyl)amino]propanoate (153 mg, 0.42 mmol), toluene (1 mL), and H₂O (0.2 mL) at rt under an atmosphere of N₂. The mixture was heated to 60° C. and stirred for 3 h, cooled, diluted with H₂O (10 mL) and extracted with EtOAc (10 mL×3). The combined organic layers were washed with brine (3×10 mL), dried over anhydrous Na₂SO₄, and filtered. The filtrate was concentrated under reduced pressure and the residue was purified by silica gel column chromatography to give methyl (2S)-2-[(tert-butoxycarbonyl)amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoate (72 mg, 54% yield) as a solid. LCMS (ESI): m/z: [M+H] calc'd for C₃₄H₄₄N₄O₆S 636.3; found 637.2.

Step 3. A mixture of methyl (2S)-2-[(tert-butoxycarbonyl)amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoate (40 mg, 0.06 mmol) and LiOH·H₂O (unspecified) in THF (1 mL) and H₂O (0.2 mL) was stirred at rt under an atmosphere of N₂ for 2 h. The mixture was acidified to pH 5 with aqueous NaHSO₄ and extracted with EtOAc (3×10 mL). The combined organic layers were washed with brine (3×10 mL), dried over anhydrous Na₂SO₄, filtered, and concentrated under reduced pressure to give (2S)-2-((tert-butoxycarbonyl)amino)-3-(4-(1-ethyl-3-(3-hydroxy-2,2-dimethylpropyl)-2-(2-(methoxymethyl)pyridin-3-yl)-1H-indol-5-yl)thiazol-2-yl)propanoic acid. The crude product was used in the next step directly without further purification. LCMS (ESI): m/z: [M+H] calc'd for C₃₃H₄₂N₄O₆S 622.3; found 623.3.

Step 4. Methyl (3S)-1-((2S)-2-((tert-butoxycarbonyl)amino)-3-(4-(1-ethyl-3-(3-hydroxy-2,2-dimethylpropyl)-2-(2-(methoxymethyl)pyridin-3-yl)-1H-indol-5-yl)thiazol-2-yl)propanoyl)hexahydropyridazine-3-carboxylate was synthesized in a manner similar to methyl (3S)-1-[(2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylate except (2S)-2-[(tert-butoxycarbonyl)amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoic acid was substituted with (2S)-2-((tert-butoxycarbonyl)amino)-3-(4-(1-ethyl-3-(3-hydroxy-2,2-dimethylpropyl)-2-(2-(methoxymethyl)pyridin-3-yl)-1H-indol-5-yl)thiazol-2-yl)propanoic acid. LCMS (ESI): m/z: [M+H] calc'd for C₃₉H₅₂N₆O₇S 748.4; found 749.4.

Step 5. (3S)-1-[(2S)-2-[(tert-butoxycarbonyl)amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoyl]-1,2-diazinane-3-carboxylic acid was synthesized in a manner similar to (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid except methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate was substituted with methyl (3S)-1-((2S)-2-((tert-butoxycarbonyl)amino)-3-(4-(1-ethyl-3-(3-hydroxy-2,2-dimethylpropyl)-2-(2-(methoxymethyl)pyridin-3-yl)-1H-indol-5-yl)thiazol-2-yl)propanoyl)hexahydropyridazine-3-carboxylate. LCMS (ESI): m/z: [M+H] calc'd for C₃₈H₅₀N₆O₇S 734.3; found 735.4.

Step 6. Tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate was synthesized in a manner similar to tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate except (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid was substituted with (3S)-1-[(2S)-2-[(tert-butoxycarbonyl)amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoyl]-1,2-diazinane-3-carboxylic acid. LCMS (ESI): m/z: [M+H] calc'd for C₃₈H₄₈N₆O₆S 716.3; found 717.3.

Step 7. (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione was synthesized in a manner similar to (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2(2,4)-thiazola-1(5,3)-indola-6(1,3)-pyridazinacycloundecaphane-5,7-dione except tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2(2,4)-thiazola-1(5,3)-indola-6(1,3)-pyridazinacycloundecaphane-4-yl)carbamate was substituted with tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate. LCMS (ESI): m/z: [M+Na] calc'd for C₃₃H₄₀N₆O₄SNa 639.3; found 640.3.

Step 8. (2S)—N-[(7S,13S)-21-ethyl-20-[2-(methoxymethyl)pyridin-3-yl]-17,17-dimethyl-8,14-dioxo-15-oxa-4-thia-9,21,27,28-tetraazapentacyclo[17.5.2.1^{2,5}.1^{9,13}.0^{22,26}]octacosa-1(25),2,5(25),19,22(26),23-hexaen-7-yl]-3-methyl-2-{N-methyl-1-[(3S)-1-(prop-2-enoyl)pyrrolidin-3-yl]formamido}butanamide was synthesized in a manner similar to (2S)—N-[(7S,13S)-21-ethyl-20-[2-(methoxymethyl)pyridin-3-yl]-17,17-dimethyl-8,14-dioxo-15-oxa-3-thia-9,21,27,28-tetraazapentacyclo[17.5.2.1^{2,5}.1^{9,13}.0^{22,26}]octacosa-1(25),2(25),4,19,22(26),23-hexaen-7-yl]-3-methyl-2-{N-methyl-1-[(3S)-1-(prop-2-enoyl)pyrrolidin-3-yl]formamido}butanamide except (6³S,4S)-4-amino-1¹-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane- 5,7-dione was substituted with (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione. LCMS (ESI): m/z: [M+H] calc'd for C₄₇H₆₀N₈O₇S 880.4; found 881.5; ¹H NMR (400 MHz, DMSO-d₆) δ 8.70 (dt, J=16.2, 8.1 Hz, 1H), 8.54 (ddd, J=6.6, 4.7, 1.7 Hz, 1H), 8.50 (m, 1H), 7.96 (d, J=7.8 Hz, 1H), 7.88 (t, J=2.1 Hz, 2H), 6.70-6.57 (m, 2H), 6.24-6.13 (m, 2H), 5.75 (m, 1H), 5.55 (t, J=7.3 Hz, 1H), 5.46 (d, J=8.5 Hz, 1H), 5.14 (d, J=13.0 Hz, 1H), 4.84-4.75 (m, 1H), 4.35 (d, J=10.7 Hz, 1H), 4.28-4.19 (m, 4H), 3.91 (s, 3H), 3.87 (dd, J=10.4, 8.1 Hz, 1H), 3.78-3.70 (m, 2H), 3.63 (t, J=8.8 Hz, 2H), 3.61-3.49 (m, 2H), 2.87 (d, J=1.1 Hz, 2H), 2.79 (s, 1H), 2.38 (s, 1H), 2.18 (s, 1H), 2.13 (d, J=10.7 Hz, 4H), 1.96 (s, 2H), 1.81 (s, 1H), 1.53 (s, 2H), 1.11 (t, J=7.1 Hz, 2H), 0.99-0.89 (m, 7H), 0.93-0.81 (m, 2H), 0.78 (d, J=6.6 Hz, 2H), 0.28 (s, 3H).

### Example A71. Synthesis of (2S)-2-(1-{1-[(2E)-4-(dimethylamino)but-2-enoyl]azetidin-3-yl}-N-methylformamido)-N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methylbutanamide

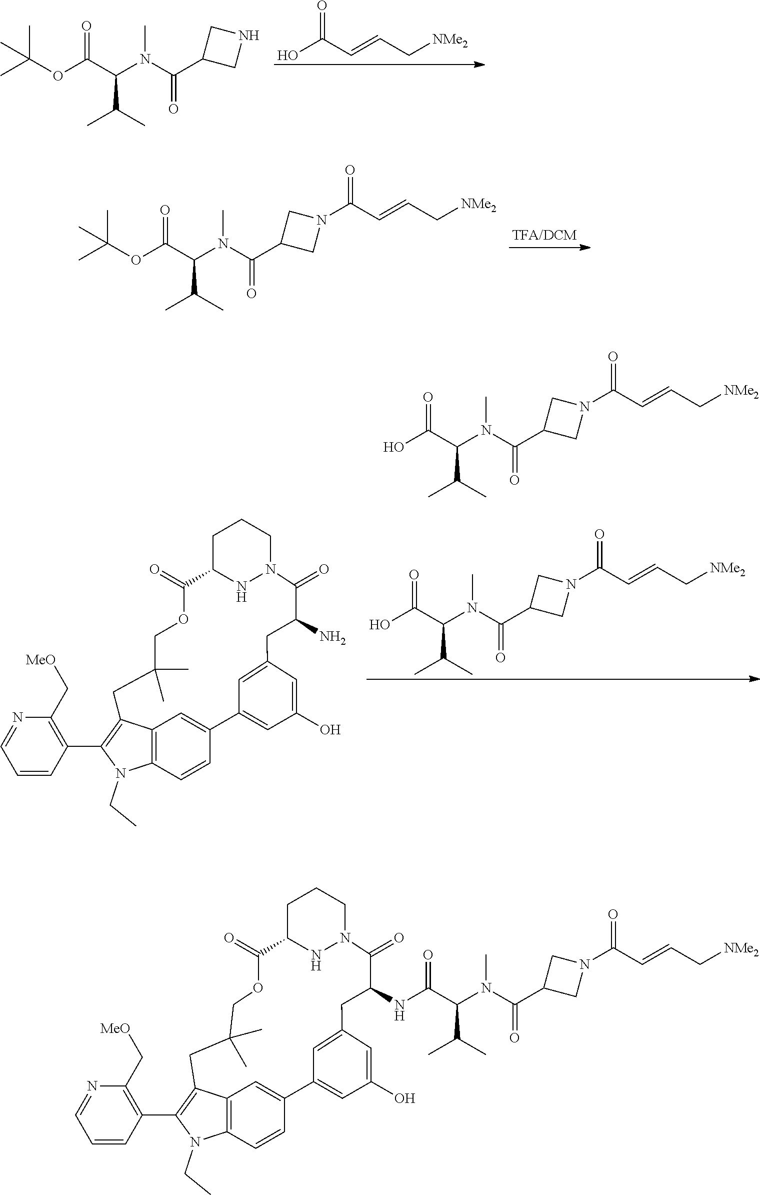

Step 1. To a mixture of tert-butyl N-(azetidine-3-carbonyl)-N-methyl-L-valinate (350 mg, 1.3 mmol) and (2E)-4-(dimethylamino)but-2-enoic acid (201 mg, 1.56 mmol) in DCM (8 mL) at 5° C. was added a solution of T3P, 50% in EtOAc (827 mg, 2.6 mmol) and DIPEA (1.7 g, 13 mmol) in DCM (2 mL). The mixture was stirred for 1 h, then diluted with EtOAc (20 mL) and H₂O (20 mL). The aqueous and organic layers were separated and the organic layer was washed with H₂O (3×10 mL), brine (10 mL), dried over anhydrous Na₂SO₄, and filtered. The filtrate was concentrated under reduced pressure and the residue was purified by prep-HPLC to give tert-butyl (E)-N-(1-(4-(dimethylamino)but-2-enoyl)azetidine-3-carbonyl)-N-methyl-L-valinate (200 mg, 39% yield) as a solid. LCMS (ESI): m/z: [M+H] calc'd for C₂₀H₃₅N₃O₄ 381.3; found 382.3.

Step 2. To a mixture of tert-butyl (E)-N-(1-(4-(dimethylamino)but-2-enoyl)azetidine-3-carbonyl)-N-methyl-L-valinate (190 mg, 0.32 mmol) in DCM (3 mL) at rt was added TFA (1 mL). The mixture was stirred at rt for 1 h, then concentrated under reduced pressure to give (E)-N-(1-(4-(dimethylamino)but-2-enoyl)azetidine-3-carbonyl)-N-methyl-L-valine (190 mg, 90%) as a solid, which was used directly in the next step without further purification. LCMS (ESI): m/z: [M+H] calc'd for C₁₆H₂₇N₃O₄ 325.2; found 326.2.

Step 3. To a mixture of (6³S,4S)-4-amino-1¹-ethyl-2⁵-hydroxy-1²-(2-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (172 mg, 0.27 mmol) and (E)-N-(1-(4-(dimethylamino)but-2-enoyl)azetidine-3-carbonyl)-N-methyl-L-valine (105 mg, 0.32 mmol) in DMF (2 mL) at 5° C. was added a mixture of HATU (133 mg, 0.297 mmol) and DIPEA (348 mg, 2.7 mmol) in DMF (1 mL). The mixture was stirred for 1 h, then diluted with EtOAc (20 mL) and H₂O (20 mL). The aqueous and organic layers were separated, and the organic layer was washed with H₂O (3×10 mL), brine (10 mL), dried over anhydrous Na₂SO₄, and filtered. The filtrate was concentrated under reduced pressure and the residue was purified by prep-TLC to give (2S)-2-(1-{1-[(2E)-4-(dimethylamino)but-2-enoyl]azetidin-3-yl}-N-methylformamido)-N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methylbutanamide (4.8 mg, 2% yield over 2 steps) as a solid. LCMS (ESI): m/z: [M+H] calc'd for C₅₂H₆₈N₈O₈ 932.5; found 933.5; ¹H NMR (400 MHz, CD₃OD) δ 8.71 (d, J=3.2 Hz, 1H), 8.50 (s, 1.5H), 8.08-7.85 (m, 2H), 7.65-7.44 (m, 3H), 7.32-7.14 (m, 1H), 7.07-6.95 (m, 1H), 6.80 (dt, J=22.1, 6.8 Hz, 1H), 6.55 (d, J=35.8 Hz, 1H), 6.30 (d, J=15.4 Hz, 1H), 5.56 (dd, J=13.8, 6.7 Hz, 1H), 4.76 (dd, J=19.8, 10.5 Hz, 1H), 4.54 (dd, J=15.9, 7.5 Hz, 2H), 4.48-4.38 (m, 2H), 4.36-4.23 (m, 3H), 4.22-4.14 (m, 1H), 3.96 (qd, J=15.6, 7.9 Hz, 3H), 3.77 (ddd, J=25.8, 23.4, 11.9 Hz, 2H), 3.58 (dd, J=17.2, 8.3 Hz, 2H), 3.38 (s, 2H), 3.25-3.11 (m, 3H), 3.05-2.94 (m, 1H), 2.94-2.81 (m, 4H), 2.73 (dd, J=20.9, 11.0 Hz, 1H), 2.45 (d, J=6.9 Hz, 5H), 2.32-2.07 (m, 3H), 1.92 (d, J=13.2 Hz, 1H), 1.72 (s, 1H), 1.64-1.51 (m, 1H), 1.18 (t, J=7.0 Hz, 2H), 1.00 (ddd, J=14.6, 11.8, 8.5 Hz, 6H), 0.92-0.81 (m, 4H), 0.55-0.41 (m, 3H).

### Example A67. Synthesis of (2E)-4-(dimethylamino)-N-(6-{[(1S)-1-{[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl](methyl)carbamoyl}pyridin-3-yl)but-2-enamide

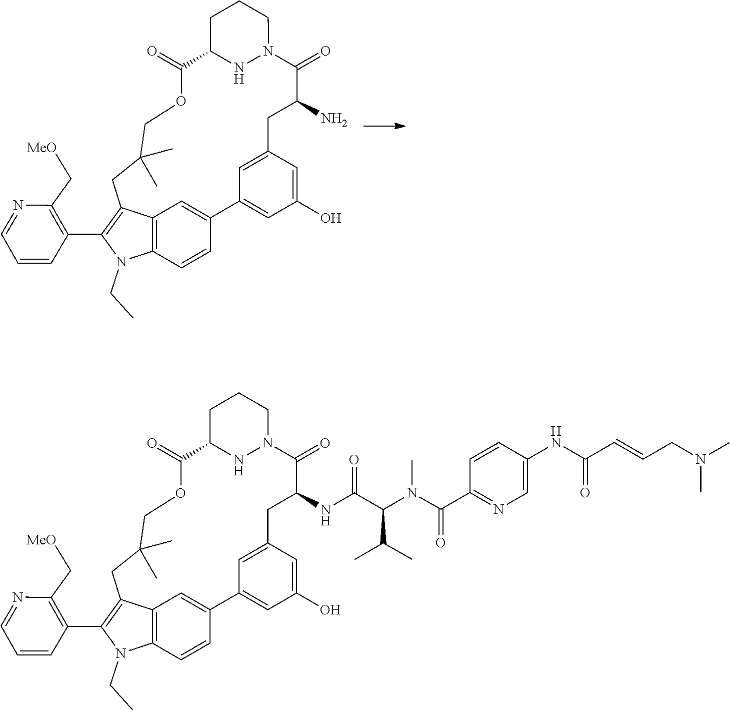 [SMILES: CCn1c(-c2cccnc2COC)c2c3cc(ccc31)-c1cc(O)cc(c1)C[C@H](N)C(=O)N1CCC[C@H](N1)C(=O)OCC(C)(C)C2 ; CCn1c(-c2cccnc2COC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2]

Step 1. To a mixture of (6³S,4S)-4-amino-1¹-ethyl-2⁵-hydroxy-1²-(2-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione TFA salt (225 mg, 0.28 mmol) and (E)-N-(5-(4-(dimethylamino)but-2-enamido)picolinoyl)-N-methyl-L-valine TFA salt (260 mg crude, 0.56 mmol) in DMF (5 mL) at 0° C. were added DIPEA (0.46 mL, 2.8 mmol) followed by HATU (140 mg, 0.36 mmol). The mixture was stirred at 0-10° C. for 1 h, then concentrated under reduced pressure and the residue was purified by prep-HPLC to give (2E)-4-(dimethylamino)-N-(6-{[(1S)-1-{[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl](methyl)carbamoyl}pyridin-3-yl)but-2-enamide TFA salt (23.3 mg, 8% yield over 2 steps) as a solid. LCMS (ESI): m/z: [M+Na] calc'd for C₅₄H₆₇N₉O₈Na 992.5; found 992.4; ¹H NMR (400 MHz, CD₃OD) δ 9.05 (d, J=2.5 Hz, 1H), 8.85-8.71 (m, 1H), 8.43 (ddd, J=33.3, 18.0, 2.6 Hz, 2H), 8.01-7.87 (m, 2H), 7.83-7.70 (m, 1H), 7.60-7.47 (m, 2H), 7.31-7.19 (m, 1H), 7.07-6.90 (m, 2H), 6.70-6.36 (m, 3H), 5.81-5.61 (m, 1H), 4.50-4.20 (m, 4H), 4.01-3.68 (m, 3H), 3.64-3.35 (m, 5H), 3.27-3.08 (m, 3H), 3.04-2.44 (m, 1H), 2.36-2.10 (m, 3H), 1.93 (d, J=13.0 Hz, 1H), 1.61 (dd, J=34.3, 21.6 Hz, 3H), 1.39-1.16 (m, 3H), 1.12-0.81 (m, 6H), 0.78-0.45 (m, 6H).

### Example A54. Synthesis of (2S)-2-{1-[(3S)-1-[(2E)-4-(dimethylamino)but-2-enoyl]pyrrolidin-3-yl]-N-methylformamido}-N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methylbutanamide

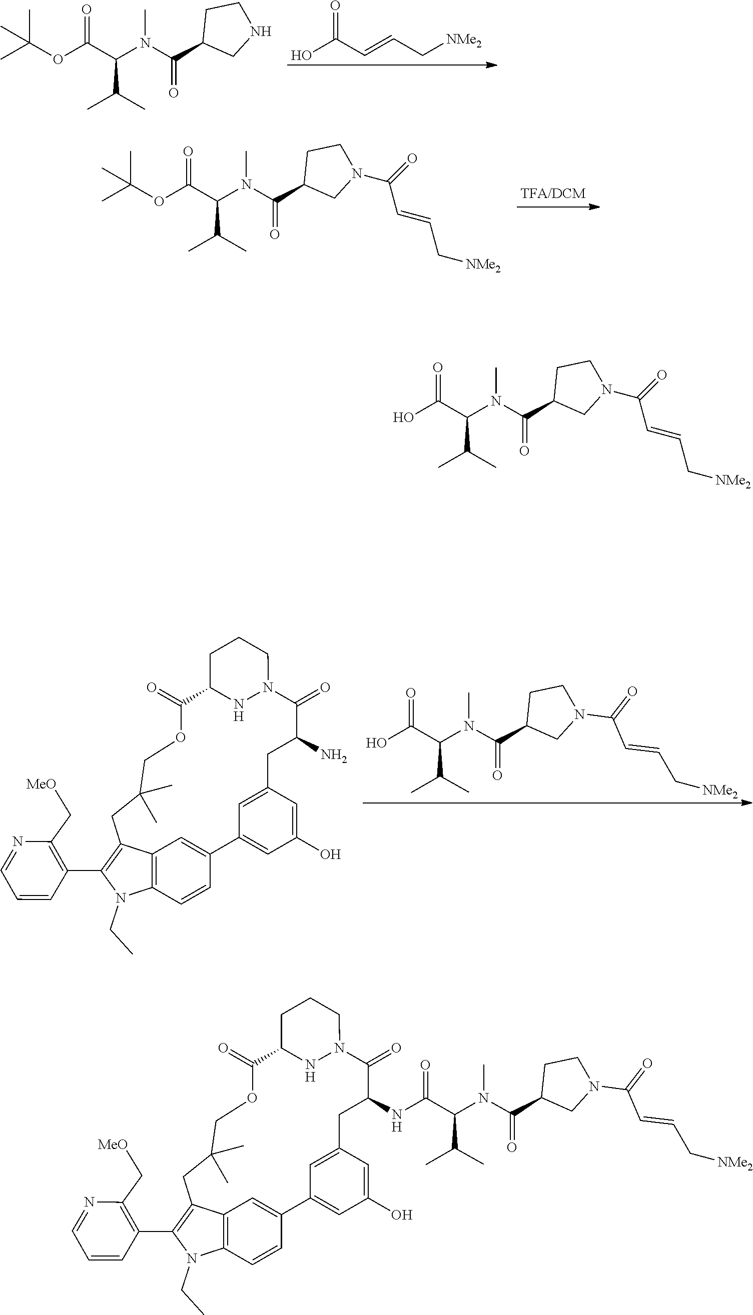

Step 1. To a mixture of tert-butyl N-methyl-N—((S)-pyrrolidine-3-carbonyl)-L-valinate (210 mg, 0.73 mmol) in DMF (4 mL) at rt were added 4-(dimethylamino)-4-methylpent-2-ynoic acid (450 mg, 2.9 mmol), DIPEA (1.2 mL, 7.3 mmol), and HATU (332 mg, 0.88 mmol). The mixture was stirred at rt for 1 h then diluted with EtOAc, and the mixture washed with H₂O, brine, dried over Na₂SO₄, and filtered. The filtrate was concentrated under reduced pressure and the residue was purified by silica gel column chromatography to give tert-butyl N—((S)-1-(4-(dimethylamino)-4-methylpent-2-ynoyl)pyrrolidine-3-carbonyl)-N-methyl-L-valinate (140 mg, 45% yield) as an oil. LCMS (ESI): m/z: [M+H] calc'd for C₂₃H₃₉N₃O₄ 421.3; found 422.3.

Step 2. A mixture of tert-butyl N—((S)-1-(4-(dimethylamino)-4-methylpent-2-ynoyl)pyrrolidine-3-carbonyl)-N-methyl-L-valinate (130 mg, 0.31 mmol) in DCM (2 mL) and TFA (1 mL) was stirred at rt for 90 min. The mixture was concentrated under reduced pressure to give N—((S)-1-(4-(dimethylamino)-4-methylpent-2-ynoyl)pyrrolidine-3-carbonyl)-N-methyl-L-valine TFA salt (150 mg) as an oil, which was used directly in the next step without further purification. LCMS (ESI): m/z: [M+H] calc'd for C₁₉H₃₁N₃O₄ 365.2; found 366.2.

Step 3. (3S)-1-(4-(dimethylamino)-4-methylpent-2-ynoyl)-N-((2S)-1-(((6³S,4S)-1¹-ethyl-2⁵-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide TFA salt was synthesized in a manner similar to 1-acryloyl-N-((2S)-1-(((6³S,4S)-1¹-ethyl-2⁵-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide except (2S)-2-{1-[(3S)-1-[(2E)-4-(dimethylamino)but-2-enoyl]pyrrolidin-3-yl]-N-methylformamido}-N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methylbutanamide TFA salt. (120 mg, 54% yield over 2 steps) as a solid. ¹H-NMR (400 MHz, CD₃OD) δ 8.76-8.68 (m, 1H), 8.44 (s, 1H), 8.02-7.94 (m, 1H), 7.94-7.84 (m, 1H), 7.65-7.43 (m, 3H), 7.27-7.14 (m, 1H), 7.06-6.96 (m, 1H), 6.65-6.48 (m, 1H), 5.62-5.46 (m, 1H), 4.81-4.57 (m, 1H), 4.46-4.22 (m, 3H), 4.10-3.35 (m, 1H), 3.26-2.93 (m, 6H), 2.91-2.51 (m, 4H), 2.42-2.09 (m, 9H), 1.95-1.87 (m, 1H), 1.85-1.40 (m, 6H), 1.38-1.10 (m, 6H), 1.07-0.81 (m, 9H), 0.56-0.38 (m, 3H). LCMS (ESI): m/z [M+H] C₅₂H₆₈N₈O₈ found 947.7.

### Example A95. Synthesis of (2S)—N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-[4-(morpholin-4-yl)but-2-ynoyl]pyrrolidin-3-yl]formamido}butanamide

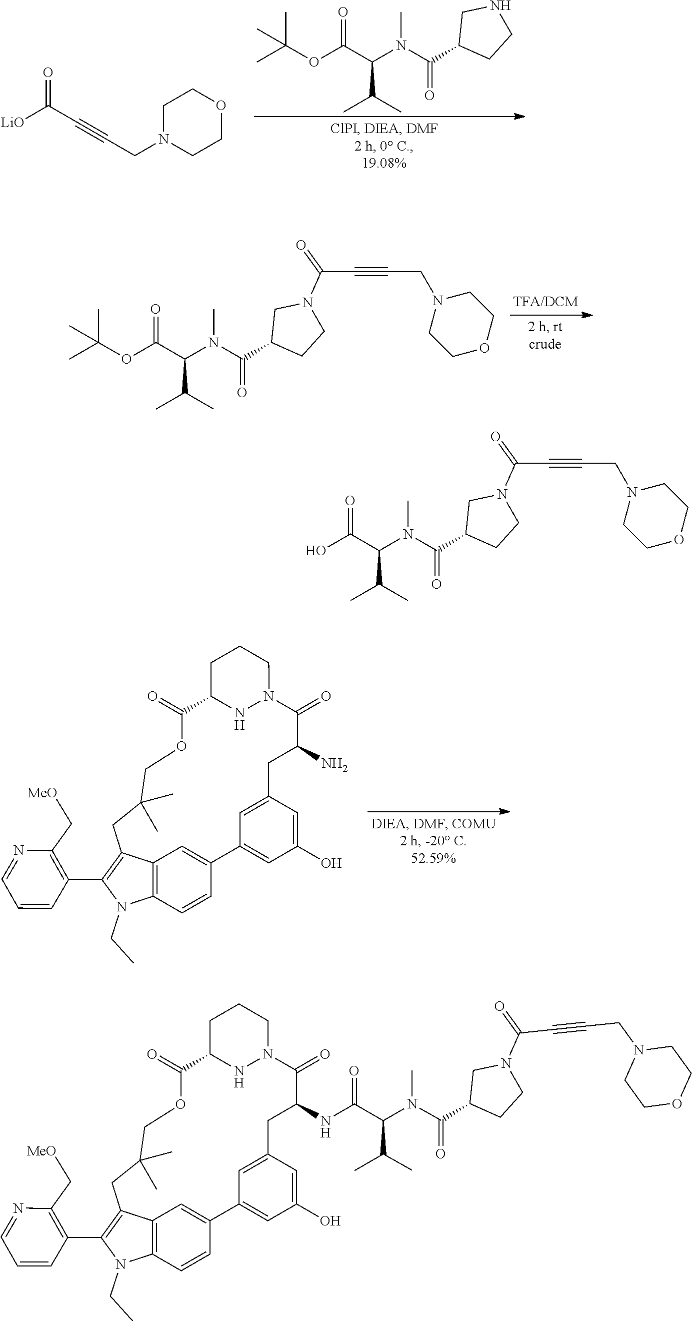

Step 1. A mixture of tert-butyl (2S)-3-methyl-2-[N-methyl-1-(3S)-pyrrolidin-3-ylformamido]butanoate (500 mg, 1.8 mmol), 4-(morpholin-4-yl)but-2-ynoic acid (1.49 g, 8.8 mmol), DIPEA (682 mg, 5.3 mmol) and CIP (635 mg, 2.3 mmol) in DMF (5 mL) was stirred at 0° C. for 2 h. The mixture was concentrated under reduced pressure and the residue was purified by silica gel column chromatography to give tert-butyl N-methyl-N—((S)-1-(4-morpholinobut-2-ynoyl)pyrrolidine-3-carbonyl)-L-valinate (150 mg, 19% yield) as an oil. LCMS (ESI): m/z: [M+H] calc'd for C₂₃H₃₇N₃O₅ 435.3; found 436.5.

Step 2. A mixture of tert-butyl N-methyl-N—((S)-1-(4-morpholinobut-2-ynoyl)pyrrolidine-3-carbonyl)-L-valinate (250 mg, 0.57 mmol) in DCM (5 mL) and TFA (2.5 mL) was stirred at rt for 2 h. The mixture was concentrated under reduced pressure to give (2S)-3-methyl-2-[N-methyl-1-[(3S)-1-[4-(morpholin-4-yl)but-2-ynoyl]pyrrolidin-3-yl]formamido]butanoic acid (310 mg, crude) as an oil, which was used directly in the next step without further purification. LCMS (ESI): m/z: [M+H] calc'd for C₁₉H₂₉N₃O₅ 379.2; found 380.2.

Step 3. A mixture of (6³S,4S)-4-amino-1¹-ethyl-2⁵-hydroxy-1²-(2-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (250 mg, 0.4 mmol), DIPEA (516 mg, 4.0 mmol), (2S)-3-methyl-2-[N-methyl-1-[(3S)-1-[4-(morpholin-4-yl)but-2-ynoyl]pyrrolidin-3-yl]formamido]butanoic acid (182 mg, 0.48 mmol), and COMU (205 mg, 0.48 mmol) in DMF (3 mL) was stirred at −20° C. for 2 h. The mixture was diluted with H₂O (10 mL), then extracted with EtOAc (3×10 mL) and the combined organic layers were washed with brine (3×10 mL), dried over anhydrous Na₂SO₄, and filtered. The mixture was concentrated under reduced pressure and the residue was purified by reverse-phase silica gel column chromatography to give (2S)—N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-[4-(morpholin-4-yl)but-2-ynoyl]pyrrolidin-3-yl]formamido}butanamide (207 mg, 53% yield) as a solid. LCMS (ESI): m/z: [M+H] calc'd for C₅₅H₇₀N₈O₉ 986.5; found 987.8; ¹H NMR (400 MHz, DMSO-d₆) δ 9.39-9.28 (m, 1H), 8.74 (t, J=4.8, 1H), 8.70-8.04 (m, 1H), 7.98-7.90 (m, 1.5H), 7.82 (d, J=7.7 Hz, 0.5H), 7.63-7.46 (m, 3H), 7.26-7.10 (m, 1H), 7.03 (s, 1H), 6.58-6.43 (m, 1H), 5.44-5.30 (m, 1H), 5.06 (q, 0.5H), 4.72 (t, J=11.0, 0.5H), 4.39-4.20 (m, 3H), 4.15 (d, J=11.1 Hz, 1H), 4.09-3.85 (m, 4H), 3.66 (s, 2H), 3.65-3.58 (m, 4H), 3.58-3.55 (m, 2H), 3.55-3.48 (m, 3H), 3.47-3.41 (m, 3H), 3.31 (s, 2H), 3.10 (s, 2H), 2.92 (s, 1H), 2.89-2.65 (m, 5H), 2.68 (s, 1H), 2.45-2.38 (m, 1H), 2.29-2.24 (m, 1H), 2.23-1.99 (m, 3H), 1.82 (d, J=12.1 Hz, 1H), 1.76-1.62 (m, 1H), 1.61-1.45 (m, 1H), 1.14-1.04 (m, 2H), 1.02-0.92 (m, 3H), 0.91-0.86 (m, 3H), 0.83-0.77 (m, 3H), 0.77-0.70 (m, 2H), 0.50-0.35 (m, 3H).

### Example A145. Synthesis of two atropisomers of (2S)-2-{1-[(3S)-1-(but-2-ynoyl)pyrrolidin-3-yl]-N-methylformamido}-N-[(8S,14S,20M)-22-ethyl-4-hydroxy-21-{2-[(1S)-1-methoxyethyl]pyridin-3-yl}-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methylbutanamide

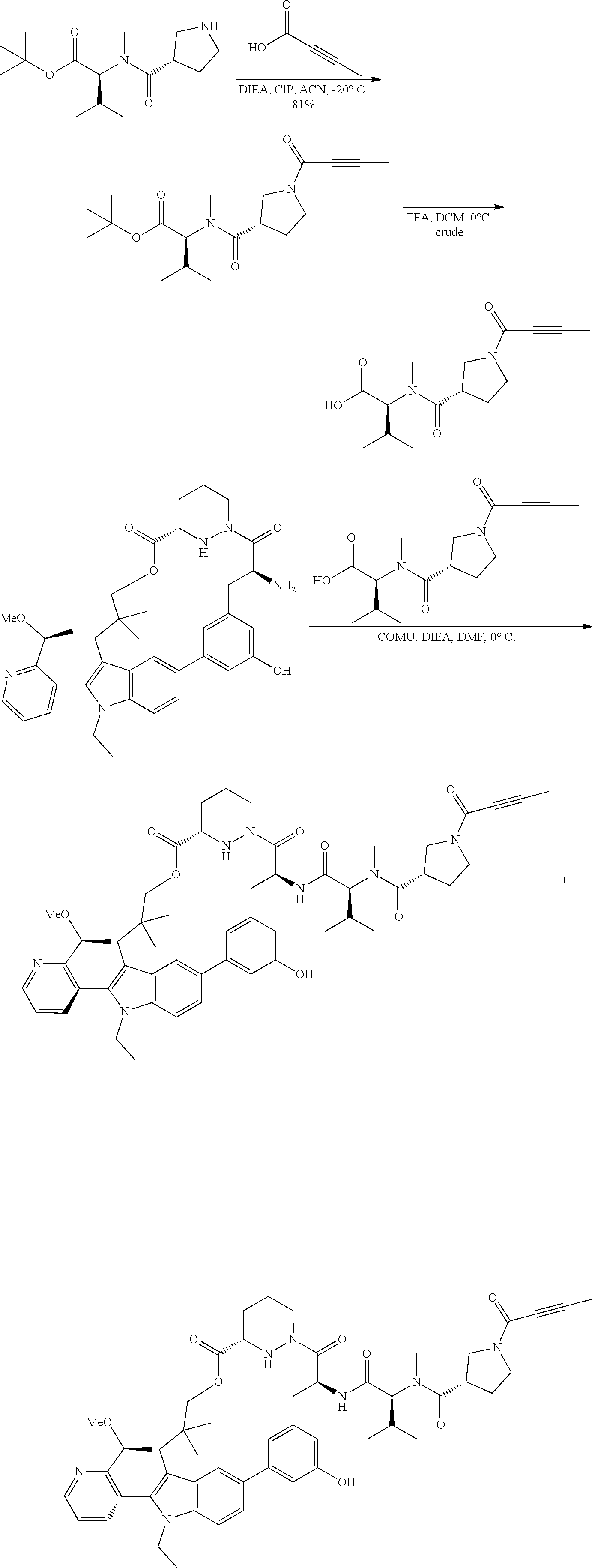

Step 1. To a mixture of but-2-ynoic acid (222 mg) and CIP (588 mg) in ACN (8 mL) at 0° C. under an atmosphere of Ar was added DIPEA (681 mg). The mixture was stirred at 0° C. then tert-butyl N-methyl-N—((S)-pyrrolidine-3-carbonyl)-L-valinate (500 mg) in ACN (3 mL) was added dropwise and the mixture stirred at 0° C. for 2 h. EtOAc was added and the mixture was washed with brine (3×20 mL), dried over anhydrous Na₂SO₄, and filtered. The filtrate was concentrated under reduced pressure and the residue was purified by silica gel column chromatography to give tert-butyl N—((S)-1-(but-2-ynoyl)pyrrolidine-3-carbonyl)-N-methyl-L-valinate as a solid. LCMS (ESI): m/z: [M+H] calc'd for C₁₉H₃₀N₂O₄ 350.2; found 352.1.

Step 2. A mixture of tert-butyl N—((S)-1-(but-2-ynoyl)pyrrolidine-3-carbonyl)-N-methyl-L-valinate (200 mg) in DCM (4 mL) and TFA (2 mL) was stirred at 0° C. for 2 h. The mixture was concentrated under reduced pressure with azeotropic removal of H₂O using toluene (4 mL×2) to give N—((S)-1-(but-2-ynoyl)pyrrolidine-3-carbonyl)-N-methyl-L-valinate as a solid. LCMS (ESI): m/z: [M+H] calc'd for C₁₅H₂₂N₂O₄ 294.2; found 295.2.

Step 3. Two atropisomers of (2S)-2-{1-[(3S)-1-(but-2-ynoyl)pyrrolidin-3-yl]-N-methylformamido}-N-[(8S,14S,20M)-22-ethyl-4-hydroxy-21-{2-[(1S)-1-methoxyethyl]pyridin-3-yl}-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methylbutanamide was synthesized in a manner similar to (2S)—N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-[4-(morpholin-4-yl)but-2-ynoyl]pyrrolidin-3-yl]formamido}butanamide except (2S)-3-methyl-2-[N-methyl-1-[(3S)-1-[4-(morpholin-4-yl)but-2-ynoyl]pyrrolidin-3-yl]formamido]butanoic acid was substituted with N—((S)-1-(but-2-ynoyl)pyrrolidine-3-carbonyl)-N-methyl-L-valinate. (43.3 mg, 12% yield) and (33 mg, 9% yield) both as solids. LCMS (ESI): m/z: [M+H] calc'd for C₅₂H₆₅N₇O₈ 915.5; found 916.7; ¹H NMR (400 MHz, DMSO-d₆) δ 9.34-9.27 (m, 1H), 8.78 (t, J=2.5 Hz, 1H), 8.68 (t, J=8.5 Hz, 0.5H), 8.20-8.11 (m, 0.6H), 7.95 (ddt, J=5.4, 3.5, 1.7 Hz, 2H), 7.63-7.60 (m, 1H), 7.61-7.49 (m, 2H), 7.13 (s, 1H), 7.03 (d, J=6.2 Hz, 1H), 6.60-6.49 (d, J=35.5 Hz, 1H), 5.43-5.39 (m, 1H), 5.12-5.00 (m, 0.7H), 4.74 (d, J=10.6 Hz, 0.4H), 4.32-4.25 (m, 1H), 4.18-3.85 (m, 5H), 3.81-3.45 (m, 8H), 3.18-3.02 (m, 5H), 2.93-2.80 (m, 4H), 2.80-2.70 (m, 2H), 2.42-2.36 (m, 1H), 2.31-2.20 (m, 1H), 2.18-1.96 (m, 6H), 1.85-1.74 (m, 1H), 1.74-1.63 (m, 1H), 1.62-1.42 (m, 1H), 1.32-1.16 (m, 4H), 1.15-1.05 (t, J=6.3 Hz, 4H), 1.04-0.95 (m, 2H), 0.95-0.85 (m, 5H), 0.68-0.52 (m, 4H), 0.52-0.37 (m, 4H). and LCMS (ESI): m/z: [M+H] calc'd for C₅₂H₆₅N₇O₈ 915.5; found 916.7; ¹H NMR (400 MHz, DMSO-d₆) δ 9.36-9.28 (m, 1H), 8.77 (dd, J=4.7, 1.8 Hz, 1H), 8.62-8.57 (m, 0.5H), 8.15-8.07 (m, 0.5H), 7.95 (s, 1H), 7.87-7.81 (m, 1H), 7.65-7.51 (m, 3H), 7.37-7.25 (m, 1H), 7.10-7.03 (m, 1H), 6.54 (d, J=35.5 Hz, 1H), 5.52-5.21 (m, 2H), 4.78-4.66 (m, 0.5H), 4.34-4.20 (m, 3H), 4.15-3.85 (m, 4H), 3.85-3.42 (m, 7H), 3.22-3.11 (m, 3H), 2.97-2.72 (m, 7H), 2.62-2.54 (m, 1H), 2.28-1.96 (m, 7H), 1.95-1.74 (m, 2H), 1.73-1.44 (m, 2H), 1.42-1.37 (m, 3H), 1.28-1.14 (m, 1H), 1.03-0.85 (m, 6H), 0.83-0.72 (m, 7H), 0.71-0.55 (m, 3H).

### Example A28. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-[N-methyl-1-[(3S)-1-(prop-2-enoyl)pyrrolidin-3-yl]formamido]butanamide

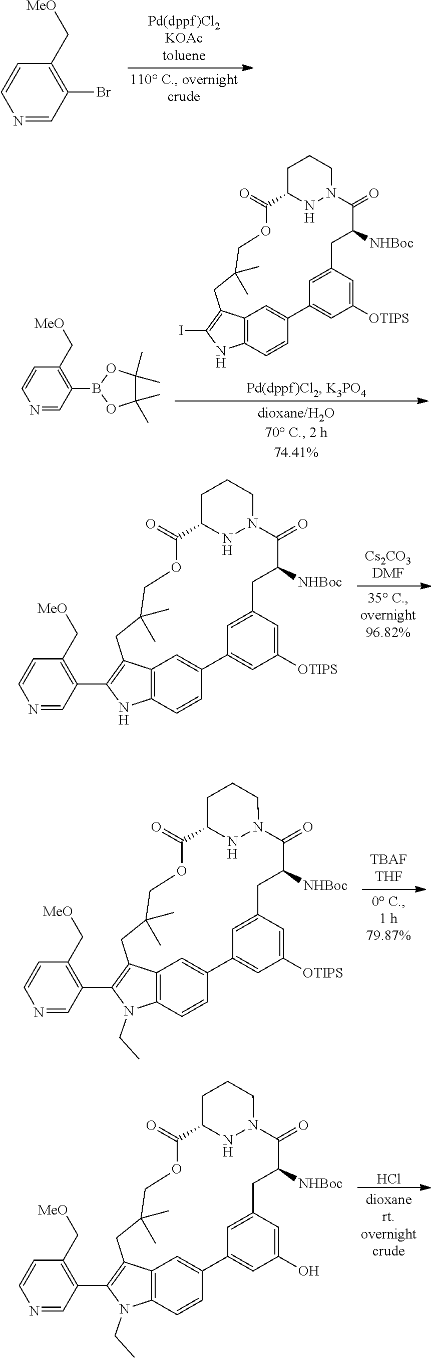

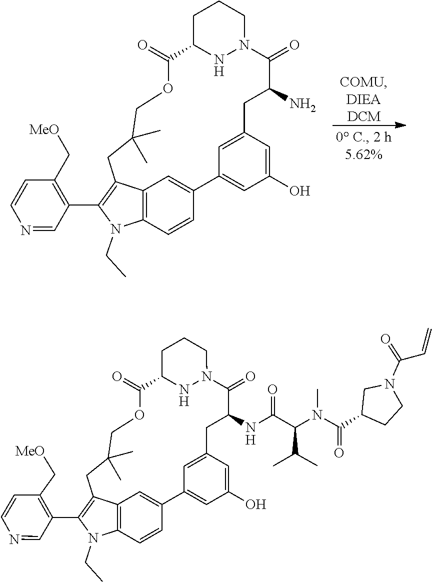 [SMILES: CCn1c(-c2cnccc2COC)c2c3cc(ccc31)-c1cc(O)cc(c1)C[C@H](N)C(=O)N1CCC[C@H](N1)C(=O)OCC(C)(C)C2 ; "COMU,DIEADCM" ; C=CC(=O)N1CC[C@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)C1]

Step 1. To a mixture of 3-bromo-4-(methoxymethyl)pyridine (1.00 g, 5.0 mmol), 4,4,5,5-tetramethyl-2-(tetramethyl-1,3,2-dioxaborolan-2-yl)-1,3,2-dioxaborolane (1.51 g, 5.9 mmol) and KOAc (1.21 g, 12.3 mmol) in toluene (10 mL) at rt under an atmosphere of Ar was added Pd(dppf)Cl₂ (362 mg, 0.5 mmol). The mixture was heated to 110° C. and stirred overnight, then concentrated under reduced pressure to give 4-(methoxymethyl)-3-(4,4,5,5-tetramethyl-1,3,2-dioxaborolan-2-yl)pyridine, which was used directly in the next step directly without further purification. LCMS (ESI): m/z: [M+H] calc'd for C₁₃H₂₀BNO₃ 249.2; found 250.3.

Step 2. To a mixture of 4-(methoxymethyl)-3-(4,4,5,5-tetramethyl-1,3,2-dioxaborolan-2-yl)pyridine (290 mg, 1.16 mmol), K₃PO₄ (371 mg, 1.75 mmol) and tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (500 mg, 0.58 mmol) in 1,4-dioxane (5 mL) and H₂O (1 mL) at rt under an atmosphere of Ar was added Pd(dppf)Cl₂ (43 mg, 0.06 mmol). The mixture was heated to 70° C. and stirred for 2 h, then H₂O added and the mixture extracted with EtOAc (2×10 mL). The combined organic layers were washed with brine (10 mL), dried over anhydrous Na₂SO₄, and filtered. The filtrate was concentrated under reduced pressure and the residue was purified by silica gel column chromatography to give tert-butyl N-[(8S,14S)-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (370 mg, 74% yield) as a foam. LCMS (ESI): m/z: [M+H] calc'd for C₄₈H₆₇N₅O₇Si 853.6; found 854.6.

Step 3. A mixture of tert-butyl N-[(8S,14S)-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (350 mg, 0.41 mmol), Cs₂CO₃ (267 mg, 0.82 mmol) and EtI (128 mg, 0.82 mmol) in DMF (4 mL) was stirred at 35° C. overnight. H₂O was added and the mixture was extracted with EtOAc (2×15 mL). The combined organic layers were washed with brine (15 mL), dried over anhydrous Na₂SO₄, and filtered. The filtrate was concentrated under reduced pressure and the residue was purified by silica gel column chromatography to give tert-butyl N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (350 mg, 97% yield) as an oil. LCMS (ESI): m/z: [M+H] calc'd for C₅₀H₇₁N₅O₇Si 881.5; found 882.6.

Step 4. A mixture of tert-butyl N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (350 mg, 0.4 mmol) and 1M TBAF in THF (0.48 mL, 0.480 mmol) in THF (3 mL) at 0° C. under an atmosphere of Ar was stirred for 1 h. The mixture was concentrated under reduced pressure and the residue was purified by silica gel column chromatography to give tert-butyl N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (230 mg, 80% yield) as an oil. LCMS (ESI): m/z: [M+H] calc'd for C₄₁H₅₁N₅O₇ 725.4; found 726.6.

Step 5. To a mixture of tert-butyl I-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (200 mg, 0.28 mmol) in 1,4-dioxane (2 mL) at 0° C. under an atmosphere of Ar was added 4M HCl in 1,4-dioxane (2 mL, 8 mmol). The mixture was allowed to warm to rt and was stirred overnight, then concentrated under reduced pressure to give (8S,14S)-8-amino-22-ethyl-4-hydroxy-21-[4-(methoxymethyl)pyridin-3-yl]-18,18-dimethyl-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaene-9,15-dione (200 mg). LCMS (ESI): m/z: [M+H] calc'd for C₃₆H₄₃N₅O₅ 625.3; found 626.5.

Step 6. To a mixture of (2S)-3-methyl-2-[N-methyl-1-[(3S)-1-(prop-2-enoyl)pyrrolidin-3-yl]formamido]butanoic acid (108 mg, 0.38 mmol) and (8S,14S)-8-amino-22-ethyl-4-hydroxy-21-[4-(methoxymethyl)pyridin-3-yl]-18,18-dimethyl-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaene-9,15-dione (200 mg, 0.32 mmol) in DCM (3 mL) at 0° C. was added DIPEA (165 mg, 1.3 mmol) and COMU (274 mg, 0.64 mmol) in portions. The mixture was stirred at 0° C. for 2 h, H₂O added and extracted with EtOAc (2×10 mL). The combined organic layers were washed with brine (10 mL), dried over anhydrous Na₂SO₄, and filtered. The filtrate was concentrated under reduced pressure and the residue was purified by silica gel column chromatography then prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-[N-methyl-1-[(3S)-1-(prop-2-enoyl)pyrrolidin-3-yl]formamido]butanamide (16 mg, 5.6% yield) as a solid. LCMS (ESI): m/z: [M+H] calc'd for C₅₀H₆₃N₇O₈ 889.5; found 890.6; ¹H NMR (400 MHz, DMSO-d₆) δ 9.33 (dd, J=9.1, 6.9 Hz, 1H), 8.79-8.46 (m, 2H), 7.93 (s, 1H), 7.68-7.58 (m, 2H), 7.53 (t, J=8.5 Hz, 1H), 7.26-6.98 (m, 2H), 6.71-6.47 (m, 2H), 6.24-6.07 (m, 1H), 5.80-5.60 (m, 1H), 5.49-5.18 (m, 1H), 4.45-4.07 (m, 4H), 4.08-3.87 (m, 3H), 3.87-3.64 (m, 4H), 3.64-3.40 (m, 5H), 3.34 (s, 2H), 3.30 (s, 2H), 3.23 (d, J=1.8 Hz, 1H), 2.94-2.74 (m, 6H), 2.16-2.01 (m, 3H), 1.82-1.47 (m, 3H), 1.08 (q, J=8.9, 8.0 Hz, 1H), 1.00-0.88 (m, 6H), 0.82 (d, J=10.8 Hz, 4H), 0.76-0.66 (m, 2H), 0.44 (d, J=14.2 Hz, 3H).

### Example A316. Synthesis of (2R)-2-(((1-(4-(dimethylamino)-4-methylpent-2-ynoyl)azetidin-3-yl)oxy)methyl)-N-((6³S4S)-1¹-ethyl-1²-(2-((S)-1-methoxyethyl)pyridin-3-yl)-10,10-dimethyl-5,7-dioxo-6¹,6²,6³,6⁴6⁵,6⁶-hexahydro-1¹H-8-oxa-1(5,3)-indola-(1,3)-pyridazina-2(1,3)-benzenacycloundecaphane-4-yl)-3-methylbutanamide

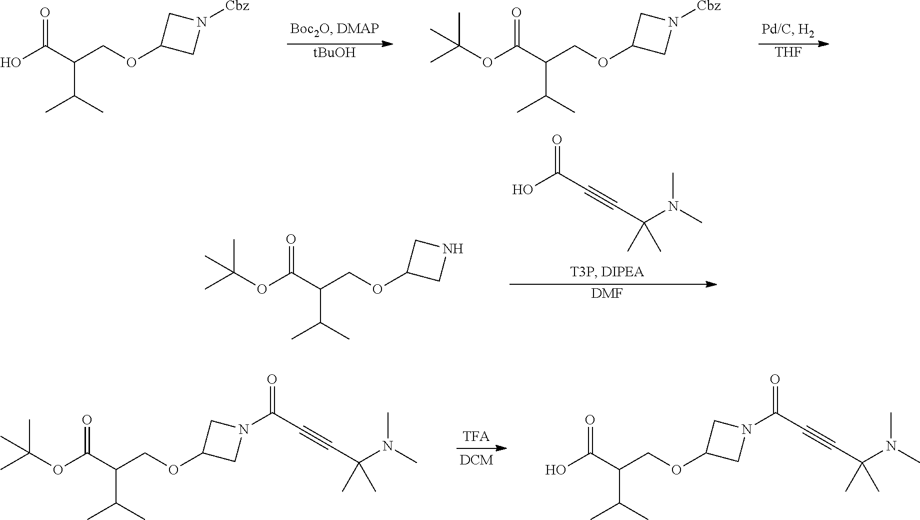

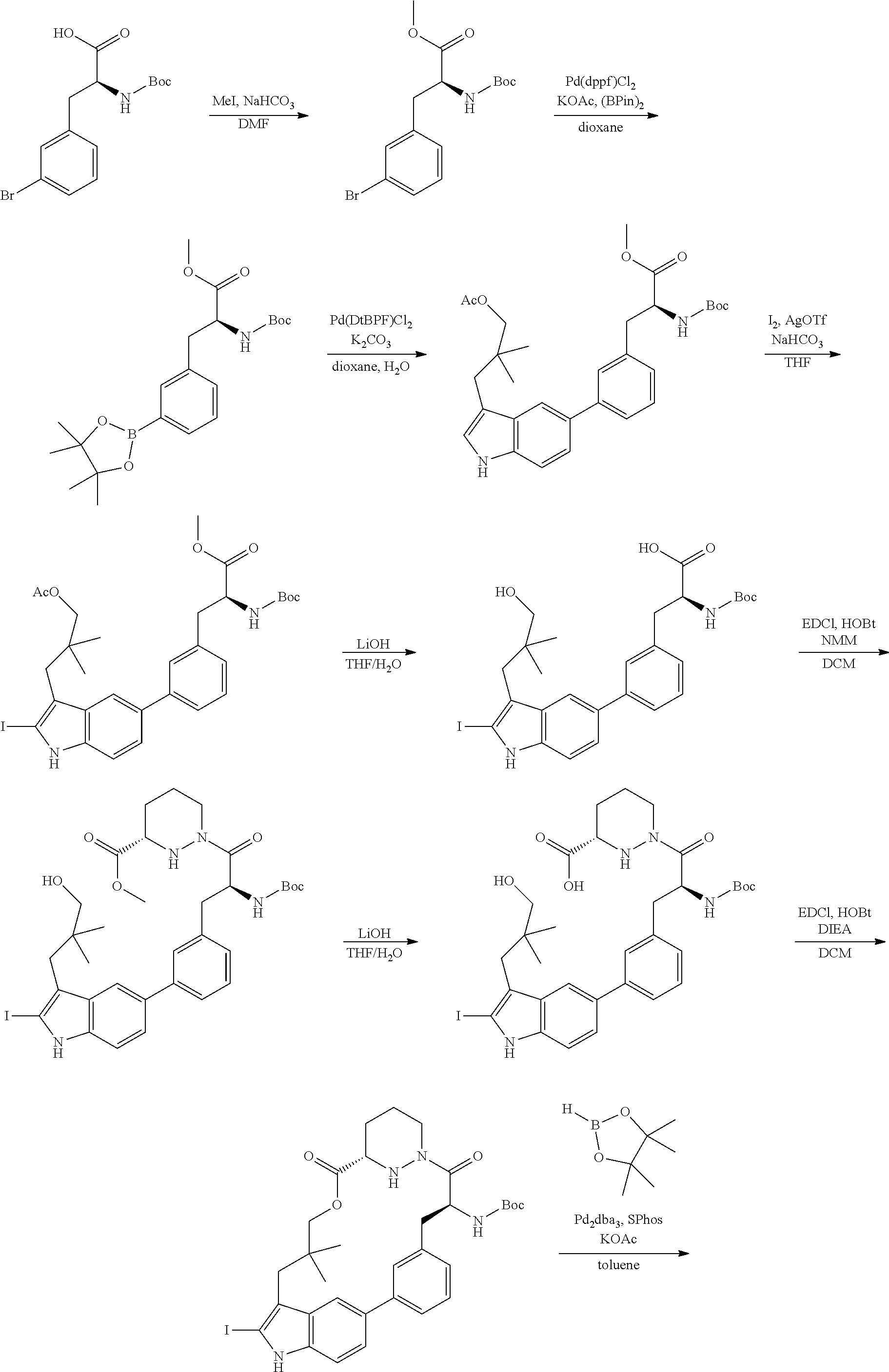

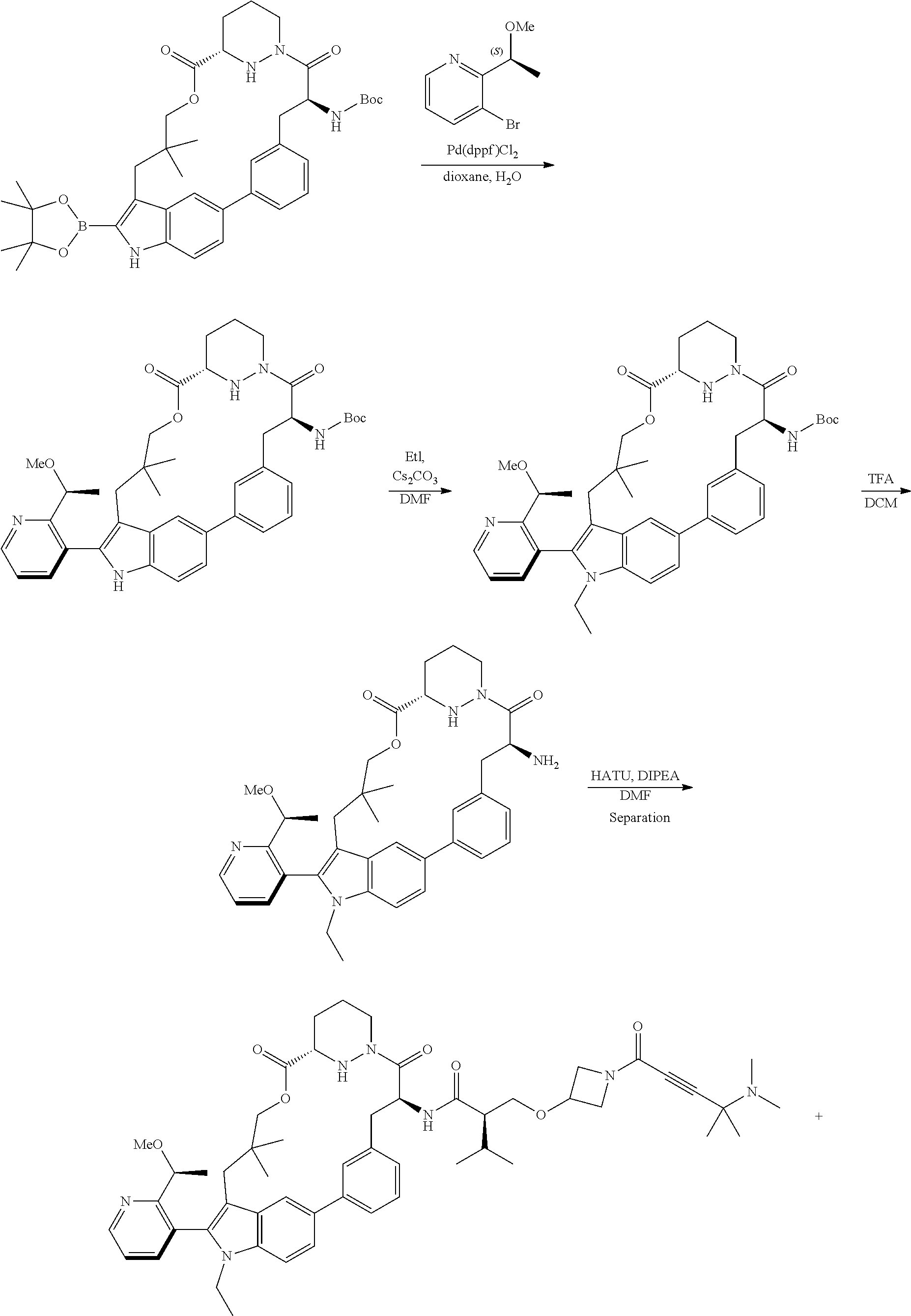

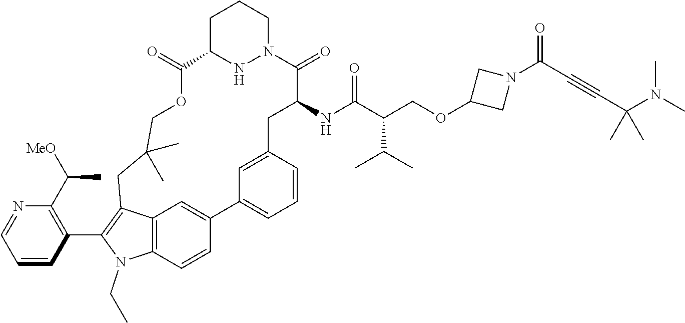

Step 1. To a mixture of 2-(((1-((benzyloxy)carbonyl)azetidin-3-yl)oxy)methyl)-3-methylbutanoic acid (650 mg, 2 mmol) and di-tert-butyl dicarbonate (883 mg, 4 mmol) in ^{t}BuOH (10 mL) was added 4-dimethylaminopyridine (124 mg, 1 mmol). The mixture was heated to 30° C. and stirred for 1 h, then diluted with H₂O (50 mL) and extracted with EtOAc (50 mL×3). The combined organic layers were concentrated under reduced pressure and the residue was purified by silica gel column chromatography to afford benzyl 3-(2-(tert-butoxycarbonyl)-3-methylbutoxy)azetidine-1-carboxylate (450 mg, 56% yield) as an oil. LCMS (ESI): m/z: [M+Na] calc'd for C₂₁H₃₁NO₅Na 400.2; found 400.2.

Step 2. A mixture of benzyl 3-(2-(tert-butoxycarbonyl)-3-methylbutoxy)azetidine-1-carboxylate (450 mg, 1.19 mmol) and Pd/C (50 mg) in THF (30 mL) was stirred for 2 h under an atmosphere of H₂ (15 psi). The mixture was filtered and the filtrate was concentrated under reduced pressure to give tert-butyl 2-((azetidin-3-yloxy)methyl)-3-methylbutanoate (300 mg, 100% yield) as an oil. LCMS (ESI): m/z: [M+H] calc'd for C₁₃H₂₆NO₃ 243.2; found 244.2; ¹H NMR (400 MHz, CDCl₃) δ 4.35-4.25 (m, 1H), 3.71-3.63 (m, 2H), 3.63-3.56 (m, 2H), 3.50 (t, J=8.0 Hz, 1H), 3.43 (dd, J=9.0, 4.0 Hz, 1H), 2.37-2.26 (m, 1H), 2.21 (br. s, 1H), 1.92-1.81 (m, 1H), 1.47 (s, 9H), 0.93 (d, J=6.8 Hz, 6H).

Step 3. To a mixture of tert-butyl 2-((azetidin-3-yloxy)methyl)-3-methylbutanoate (270 mg, 1.11 mmol), 4-(dimethylamino)-4-methylpent-2-ynoic acid (860 mg, 5.55 mmol) and DIPEA (1.56 g, 11.1 mmol) in DMF (20 mL) at 0° C. was added T3P (2.12 g, 6.7 mmol). The mixture was stirred at 0° C. for 1 h, diluted with EtOAc (200 mL), then washed with H₂O (30 mL×5), brine (30 mL), dried over anhydrous Na₂SO₄ and filtered. The filtrate was concentrated under reduced pressure and the residue was purified by silica gel column chromatography to give tert-butyl 2-(((1-(4-(dimethylamino)-4-methylpent-2-ynoyl)azetidin-3-yl)oxy)methyl)-3-methylbutanoate (200 mg, 47% yield) as an oil. LCMS (ESI): m/z: [M+H] calc'd for C₂₁H₃₆N₂O₄ 380.3; found 381.3.

Step 4. To a mixture of tert-butyl 2-(((1-(4-(dimethylamino)-4-methylpent-2-ynoyl)azetidin-3-yl)oxy)methyl)-3-methylbutanoate (190 mg, 0.5 mmol) in DCM (4 mL) was added TFA (2 mL). The mixture was stirred for 1 h, then concentrated under reduced pressure to give 2-(((1-(4-(dimethylamino)-4-methylpent-2-ynoyl)azetidin-3-yl)oxy)methyl)-3-methylbutanoic acid (162 mg, 100% yield) as an oil, which was used directly in the next step without further purification. LCMS (ESI): m/z: [M+H] calc'd for C₁₇H₂₆N₂O₄ 324.2; found 325.3.

Step 5. To a solution of (2S)-3-(3-bromophenyl)-2-[(tert-butoxycarbonyl)amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel chromatography (13% EtOAc/pet. ether) to give methyl (S)-3-(3-bromophenyl)-2-((tert-butoxycarbonyl)amino)propanoate (109 g, crude). LCMS (ESI): m/z: [M+Na] calc'd for C₁₅H₂₀BrNO₄ 380.05; found 380.0.

Step 6. To a stirred solution of methyl (2S)-3-(3-bromophenyl)-2-[(tert-butoxycarbonyl)amino]propanoate (108 g, 301.5 mmol) and bis(pinacolato)diboron (99.53 g, 391.93 mmol) in 1,4-dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel chromatography (5% EtOAc/pet. ether) to give methyl (S)-2-((tert-butoxycarbonyl)amino)-3-(3-(4,4,5,5-tetramethyl-1,3,2-dioxaborolan-2-yl)phenyl)propanoate (96 g, 78.6% yield). LCMS (ESI): m/z: [M+Na] calc'd for C₂₁H₃₂BNO₆ 428.22; found 428.1.

Step 7. To a mixture of methyl (2S)-2-[(tert-butoxycarbonyl)amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in 1,4-dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L) and the combined organic layers were washed with brine (3×600 mL), dried over Na₂SO₄, filtered, and concentrated under reduced pressure. The residue was purified by silica gel chromatography (20% EtOAc/pet. ether) to give methyl (S)-3-(3-(3-(3-acetoxy-2,2-dimethylpropyl)-1H-indol-5-yl)phenyl)-2-((tert-butoxycarbonyl)amino)propanoate (130 g, crude). LCMS (ESI): m/z: [M+H] calc'd for C₃₀H₃₈N₂O₆ 523.28; found 523.1.

Step 8. To a solution of methyl (2S)-3-(3-[3-[3-(acetyloxy)-2,2-dimethylpropyl]-1H-indol-5-yl]phenyl)-2-[(tert-butoxycarbonyl)amino]propanoate (95.0 g, 181.8 mmol) and iodine (36.91 g, 145.41 mmol) in THF (1 L) at −10° C. was added AgOTf (70.0 g, 272.7 mmol) and NaHCO₃ (22.9 g, 272.65 mmol). The reaction mixture was stirred for 30 min and was then quenched by the addition of sat. Na₂S₂O₃ (100 mL) at 0° C. The resulting mixture was extracted with EtOAc (3×1 L) and the combined organic layers were washed with brine (3×500 mL), dried over Na₂SO₄, filtered, and concentrated under reduced pressure. The residue was purified by silica gel chromatography (50% EtOAc/pet. ether) to give methyl (S)-3-(3-(3-(3-acetoxy-2,2-dimethylpropyl)-2-iodo-1H-indol-5-yl)phenyl)-2-((tert-butoxycarbonyl)amino)propanoate (49.3 g, 41.8% yield). LCMS (ESI) m/z: [M+H] calcd for C₃₀H₃₇IN₂O₆: 649.18; found 649.1.

Step 9. To a solution of methyl (2S)-3-(3-[3-[3-(acetyloxy)-2,2-dimethylpropyl]-2-iodo-1H-indol-5-yl]phenyl)-2-[(tert-butoxycarbonyl)amino]propanoate (60 g, 92.5 mmol) in THF (600 mL) was added a solution of LiOH·H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL) and the combined organic layers was washed with sat. brine (2×500 mL), dried over Na₂SO₄, filtered, and concentrated under reduced pressure to give (S)-2-((tert-butoxycarbonyl)amino)-3-(3-(3-(3-hydroxy-2,2-dimethylpropyl)-2-iodo-1H-indol-5-yl)phenyl)propanoic acid (45 g, 82.1% yield). LCMS (ESI): m/z: [M+Na] calc'd for C₂₇H₃₃IN₂O₆ 615.13; found 615.1.

Step 10. To a solution of (2S)-2-[(tert-butoxycarbonyl)amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBT (2.05 g, 15.19 mmol), and EDCI (19.41 g, 101.27 mmol). The reaction mixture was stirred overnight and then the mixture was washed with sat. NH₄Cl (2×200 mL) and sat. brine (2×200 mL), and the mixture was dried over Na₂SO₄, filtered, and concentrated under reduced pressure to give methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate (14 g, 38.5% yield). LCMS (ESI): m/z: [M+H] calc'd for C₃₃H₄₃IN₄O₆ 718.23; found 719.4.

Step 11. To a solution of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate (92 g, 128.0 mmol) in THF (920 mL) at 0° C. was added a solution of LiOH·H₂O (26.86 g, 640.10 mmol) in H₂O (640 mL). The reaction mixture was stirred for 2 h and was then concentrated under reduced pressure to give (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylic acid (90 g, crude). LCMS (ESI): m/z: [M+H] calc'd for C₃₂H₄₁IN₄O₆ 705.22; found 705.1.

Step 12. To a solution of of (3S)-1-[(2S)-2-[(tert-butoxycarbonyl)amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCI (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel chromatography (50% EtOAc/pet. ether) to give tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (70 g, 79.8% yield). LCMS (ESI): m/z [M+H] calc'd for C₃₂H₃₉IN₄O₅ 687.21; found 687.1.

Step 13. A 1 L round-bottom flask was charged with tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22.0 g, 32.042 mmol), toluene (300.0 mL), Pd₂(dba)₃ (3.52 g, 3.845 mmol), S-Phos (3.95 g, 9.613 mmol), and KOAc (9.43 g, 96.127 mmol) at room temperature. To the mixture was added 4,4,5,5-tetramethyl-1,3,2-dioxaborolane (26.66 g, 208.275 mmol) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford tert-butyl ((6³S,4S)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22 g, 90% yield) as a solid. LCMS (ESI): m/z: [M+H] calc'd for C₃₈H₅₁BN₄O₇ 687.3; found 687.4.

Step 14. A mixture of tert-butyl ((6³S,4S)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.0 g, 2.8 mmol), 3-bromo-2-[(1S)-1-methoxyethyl]pyridine (0.60 g, 2.8 mmol), Pd(dppf)Cl₂ (0.39 g, 0.5 mmol), and K₃PO₄ (1.2 g, 6.0 mmol) in 1,4-dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to give tert-butyl ((6³S,4S)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.5 g, 74% yield) as a solid. LCMS (ESI): m/z: [M+H] calc'd for C₄₀H₄₉N₅O₆ 695.4; found 696.5.

Step 15. To a solution of tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl) carbamate (20 g, 28.7 mmol) and Cs₂CO₃ (18.7 g, 57.5 mmol) in DMF (150 mL) at 0° C. was added a solution of ethyl iodide (13.45 g, 86.22 mmol) in DMF (50 mL). The resulting mixture was stirred overnight at 35° C. and was then diluted with H₂O (500 mL). The mixture was extracted with EtOAc (2×300 mL) and the combined organic layers were washed with brine (3×100 mL), dried over Na₂SO₄, filtered, and concentrated under reduced pressure. The residue was purified by silica gel chromatography to give tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate (4.23 g, 18.8% yield) and the atropisomer (5.78 g, 25.7% yield) as solids. LCMS (ESI): m/z: [M+H] calc'd for C₄₂H₅₃N₅O₆ 724.4; found 724.6.

Step 16. A mixture of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.3 g, 1.7 mmol) in TFA (10 mL) and DCM (20 mL) was stirred at 0° C. for 2 h. The mixture was concentrated under reduced pressure to afford (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.30 g, crude) as a solid. LCMS (ESI): m/z: [M+H] calc'd for C₃₇H₄₅N₅O₄ 623.3; found 624.4.

Step 17. To a mixture of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (258 mg, 0.41 mmol) and 2-(((1-(4-(dimethylamino)-4-methylpent-2-ynoyl)azetidin-3-yl)oxy)methyl)-3-methylbutanoic acid (162 mg, 0.5 mmol) in DMF (4 mL) at 0° C. was added a mixture of HATU (188 mg, 0.5 mmol) and DIPEA (534 mg, 4.14 mmol) in DMF (2 mL). The mixture was stirred at 0° C. for 1 h, then diluted with H₂O (30 mL) and extracted with EtOAc (30 mL×3). The combined organic layers were concentrated under reduced pressure and the residue was purified by silica gel column chromatography to give 2-(((1-(4-(dimethylamino)-4-methylpent-2-ynoyl)azetidin-3-yl)oxy)methyl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (250 mg, 64% yield) as a solid. LCMS (ESI): m/z: [M+H] calc'd for C₅₄H₇₁N₇O₇ 929.5; found 930.5; ¹H NMR (400 MHz, CD₃OD) δ 8.90-8.79 (m, 1H), 8.54-8.21 (m, 1H), 8.15-7.91 (m, 2H), 7.88-7.67 (m, 2H), 7.65-7.52 (m, 2H), 7.47-7.15 (m, 2H), 5.80-5.52 (m, 1H), 4.53-4.23 (m, 5H), 4.23-3.93 (m, 3H), 3.90-3.76 (m, 2H), 3.75-3.58 (m, 3H), 3.57-3.44 (m, 1H), 3.38 (s, 1H), 3.29-3.26 (m, 2H), 3.21-2.85 (m, 8H), 2.82-2.65 (m, 3H), 2.51-2.30 (m, 1H), 2.24-2.03 (m, 1H), 1.99-1.87 (m, 1H), 1.86-1.69 (m, 6H), 1.67-1.57 (m, 2H), 1.57-1.39 (m, 4H), 1.45-1.05 (m, 2H), 1.04-0.96 (m, 3H), 0.96-0.88 (m, 3H), 0.88-0.79 (m, 3H), 0.79-0.63 (m, 3H), 0.56 (s, 1H).

Step 18. 2-(((1-(4-(dimethylamino)-4-methylpent-2-ynoyl)azetidin-3-yl)oxy)methyl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (180 mg, 0.194 mmol) was purified by prep-HPLC to afford (2R)-2-(((1-(4-(dimethylamino)-4-methylpent-2-ynoyl)azetidin-3-yl)oxy)methyl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (41.8 mg, 23.2% yield) as a solid. LCMS (ESI): m/z [M+H] calc'd for C₅₄H₇₁N₇O₇ 930.5; found 930.5; ¹H NMR (400 MHz, MeOD) δ 8.74 (d, J=4.0 Hz, 1H), 8.53-8.30 (m, 1H), 8.10-7.95 (m, 1H), 7.94-7.80 (m, 2H), 7.68 (t, J=8.0 Hz, 1H), 7.65-7.58 (m, 1H), 7.58-7.46 (m, 2H), 7.38-7.17 (m, 2H), 5.73-5.60 (m, 1H), 4.52-4.40 (m, 1H), 4.35-4.15 (m, 4H), 4.14-3.95 (m, 2H), 3.90-3.72 (m, 3H), 3.71-3.45 (m, 4H), 3.30-3.20 (m, 3H), 3.06-2.72 (m, 5H), 2.49-2.28 (m, 4H), 2.28-2.20 (m, 3H), 2.18-2.06 (m, 1H), 2.00-1.90 (m, 1H), 1.90-1.52 (m, 4H), 1.52-1.40 (m, 5H), 1.40-1.22 (m, 4H), 1.09-0.92 (m, 8H), 0.90-0.75 (m, 3H), 0.71-0.52 (m, 3H) and (2S)-2-(((1-(4-(dimethylamino)-4-methylpent-2-ynoyl)azetidin-3-yl)oxy)methyl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (51.2 mg, 28.4% yield) as a solid. LCMS (ESI): m/z: [M+H] calc'd for C₅₄H₇₁N₇O₇ 930.5; found 930.3; ¹H NMR (400 MHz, MeOD) δ 8.74 (d, J=4.0 Hz, 1H), 8.28-8.20 (m, 0.6H), 8.11-7.98 (m, 1H), 7.97-7.80 (m, 2H), 7.73-7.48 (m, 4H), 7.46-7.36 (m, 0.4H), 7.33-7.26 (m, 1H), 7.25-7.13 (m, 1H), 5.79-5.66 (m, 1H), 4.54-4.43 (m, 1H), 4.42-4.01 (m, 7H), 3.90-3.75 (m, 2H), 3.73-3.48 (m, 4H), 3.27-3.12 (m, 3H), 3.08-2.99 (m, 1H), 2.96-2.85 (m, 2H), 2.84-2.69 (m, 2H), 2.69-2.49 (m, 6H), 2.41-2.29 (m, 1H), 2.15-2.05 (m, 1H), 1.95-1.85 (m, 1H), 1.84-1.71 (m, 1H), 1.71-1.38 (m, 1H), 1.14-1.00 (m, 3H), 1.00-0.71 (m, 9H), 0.70-0.56 (m, 3H).

### Example A427. Synthesis of 3-((1-(4-(dimethylamino)-4-methylpent-2-ynoyl)azetidin-3-yl)oxy)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)propanamide

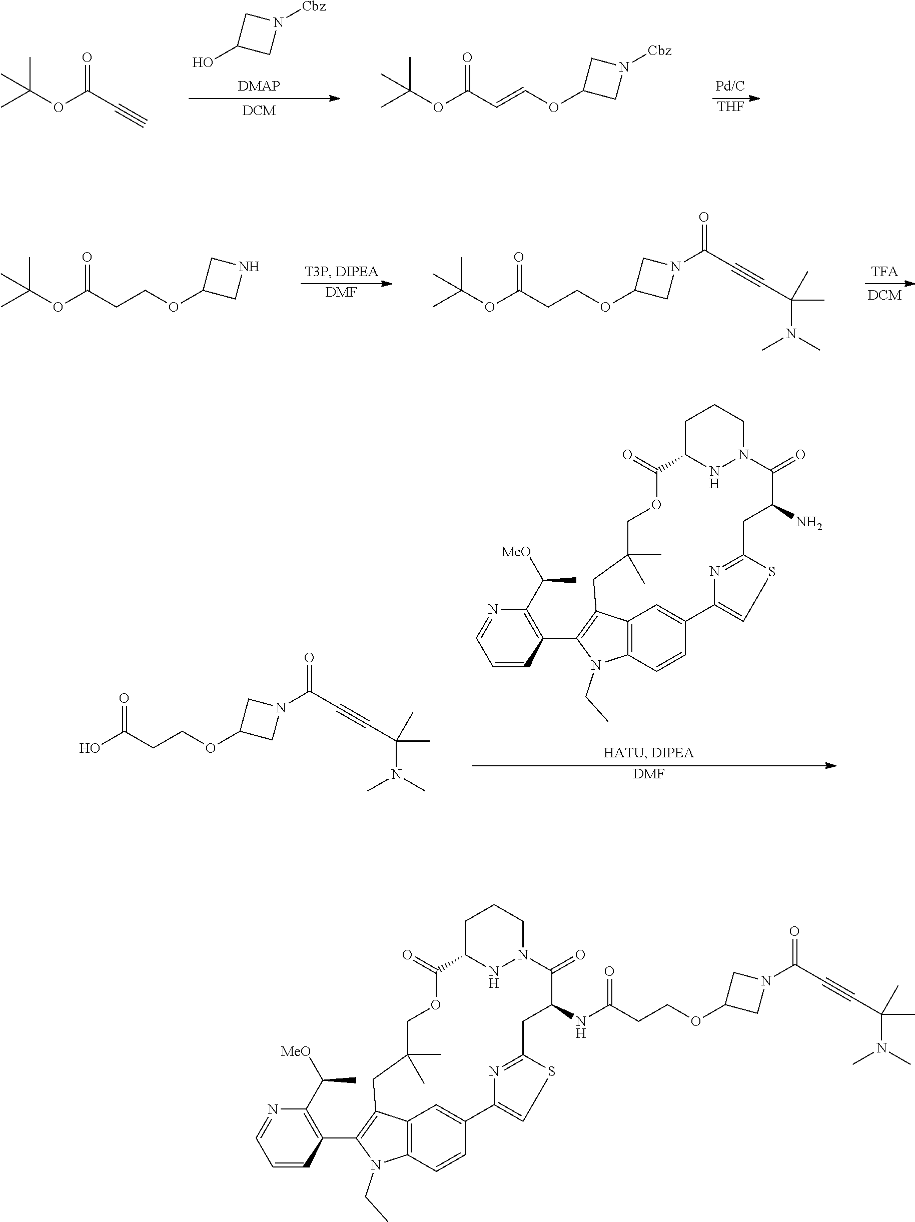

Step 1. To a mixture of tert-butyl prop-2-ynoate (5 g, 40 mmol) and [3-(3-hydroxyazetidin-1-yl)phenyl]methyl formate (4.1 g, 20 mmol) in DCM (150 mL) was added DMAP (9.8 g, 80 mmol). The mixture was stirred for 2 h, then diluted with H₂O and washed with H₂O (60 mL×3). The organic layer was dried over Na₂SO₄, filtered, the filtrate was concentrated under reduced pressure and the residue purified by silica gel column chromatography to give benzyl (E)-3-((3-(tert-butoxy)-3-oxoprop-1-en-1-yl)oxy)azetidine-1-carboxylate (6.6 g, 90% yield) as an oil. LCMS (ESI): m/z: [M+Na] calc'd for C₁₈H₂₃NO₅Na 356.2; found 358.2.

Step 2. A mixture of benzyl (E)-3-((3-(tert-butoxy)-3-oxoprop-1-en-1-yl)oxy)azetidine-1-carboxylate (1.4 g, 4 mmol) and Pd/C (200 mg) in THF (10 mL) was stirred under an atmosphere of H₂ (1 atmosphere) for 16 h. The mixture was filtered and the filtrate and was concentrated under reduced pressure to give tert-butyl 3-(azetidin-3-yloxy)propanoate, which was used directly in the next step. LCMS (ESI): m/z: [M+H] calc'd for C₁₀H₁₉NO₃ 201.1; found 202.2.

Step 3. To a mixture of tert-butyl 3-(azetidin-3-yloxy)propanoate (300 mg, 1.5 mmol) and 4-(dimethylamino)-4-methylpent-2-ynoic acid (2.3 g, 15 mmol) in DMF (15 mL) at 5° C. was added DIPEA (1.9 g, 15 mmol) and T3P (4.77 g, 7.5 mmol) dropwise. The mixture was stirred at 5° C. for 2 h, then H₂O and EtOAc (80 mL) were added. The organic and aqueous layers were separated and the organic layer was washed with H₂O (20 mL×3), brine (30 mL), dried over anhydrous Na₂SO₄ and filtered. The filtrate was concentrated under reduced pressure and the residue was purified by preparative-HPLC to afford tert-butyl 3-((1-(4-(dimethylamino)-4-methylpent-2-ynoyl)azetidin-3-yl)oxy)propanoate (60 mg, 12% yield) as an oil. LCMS (ESI): m/z: [M+H] calc'd for C₁₈H₃₀N₂O₄ 338.2; found 339.2.

Step 4. A mixture tert-butyl 3-((1-(4-(dimethylamino)-4-methylpent-2-ynoyl)azetidin-3-yl)oxy)propanoate (70 mg, 0.21 mmol) in TFA/DCM (1:3, 2 mL) was stirred at 0-5° C. for 1 h, then concentrated under reduced pressure to give 3-((1-[4-(dimethylamino)-4-methylpent-2-ynoyl]azetidin-3-yl)oxy)propanoic acid (56 mg, 95% yield) as a solid. LCMS (ESI): m/z: [M+H] calc'd for C₄H₂₂N₂O₄ 282.2; found 283.3.

Step 5. To a mixture of 3-((1-(4-(dimethylamino)-4-methylpent-2-ynoyl)azetidin-3-yl)oxy)propanoic acid (56 mg, 0.19 mmol), (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane- 5,7-dione (90 mg, 0.14 mmol) and DIPEA (200 mg, 1.9 mmol) in DMF (1 mL) at 0° C. was added HATU (110 mg, 0.38 mmol) portion-wise. The mixture was stirred at 0° C. for 1 h, then H₂O added and the mixture extracted with EtOAx (150 mL×2). The combined organic layers were washed with H₂O (150 mL) and brine (150 mL), then dried over anhydrous Na₂SO₄ and filtered. The filtrate was concentrated under reduced pressure and the residue was purified by preparative-HPLC to give 3-((1-(4-(dimethylamino)-4-methylpent-2-ynoyl)azetidin-3-yl)oxy)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)propanamide (12.6 mg, 7.5% yield) as a solid. LCMS (ESI): m/z: [M+H] calc'd for C₄₈H₆₂N₈O₇S 894.5; found 895.3; ¹H NMR (400 MHz, CD₃OD) δ 8.73 (dd, J=4.8, 1.6 Hz, 1H), 8.57 (s, 1H), 8.28 (s, 0.3H), 7.84 (m, 1H), 7.71 (m, 1H), 7.52 (m, 3H), 5.76 (dd, J=30.2, 7.8 Hz, 1H), 4.40 (m, 4H), 4.32-4.12 (m, 4H), 4.06 (dd, J=12.4, 6.0 Hz, 1H), 3.97-3.86 (m, 1H), 3.79-3.66 (m, 4H), 3.46 (dd, J=14.8, 4.8 Hz, 1H), 3.41-3.33 (m, 3H), 3.29-3.19 (m, 1H), 3.17-3.05 (m, 1H), 2.79 (m, 1H), 2.73-2.50 (m, 3H), 2.49-2.43 (m, 3H), 2.38 (s, 3H), 2.21 (dd, J=12.6, 9.6 Hz, 1H), 1.95 (d, J=12.8 Hz, 1H), 1.86-1.73 (m, 1H), 1.61 (dd, J=12.6, 3.6 Hz, 1H), 1.51 (s, 2H), 1.46-1.43 (m, 4H), 1.38-1.27 (m, 3H), 1.01-0.86 (m, 6H), 0.44 (d, J=11.6 Hz, 3H).

### Example A716. Synthesis of (3S)-1-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N-methylpyrrolidine-3-carboxamide

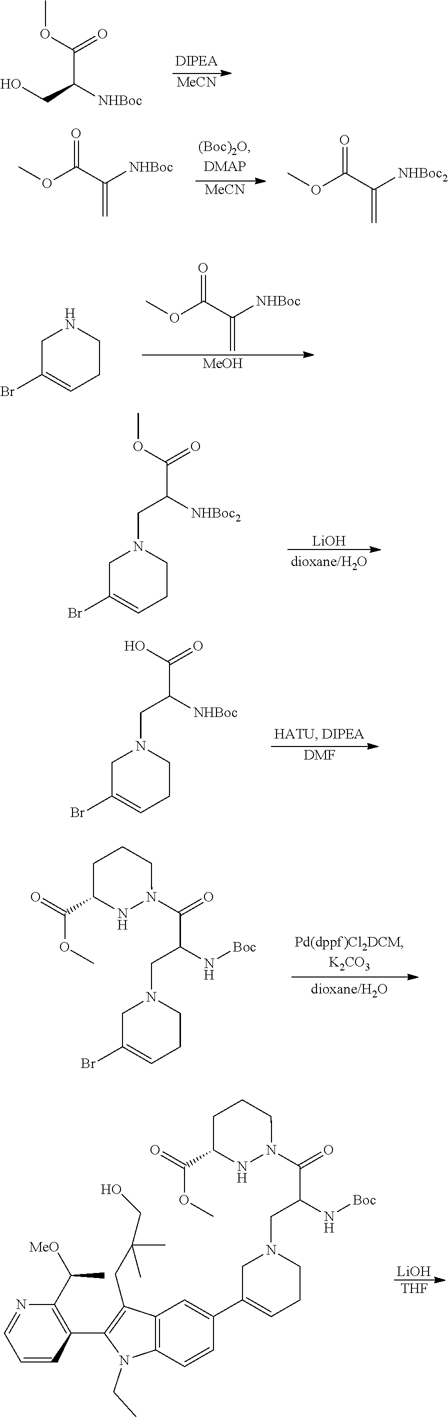

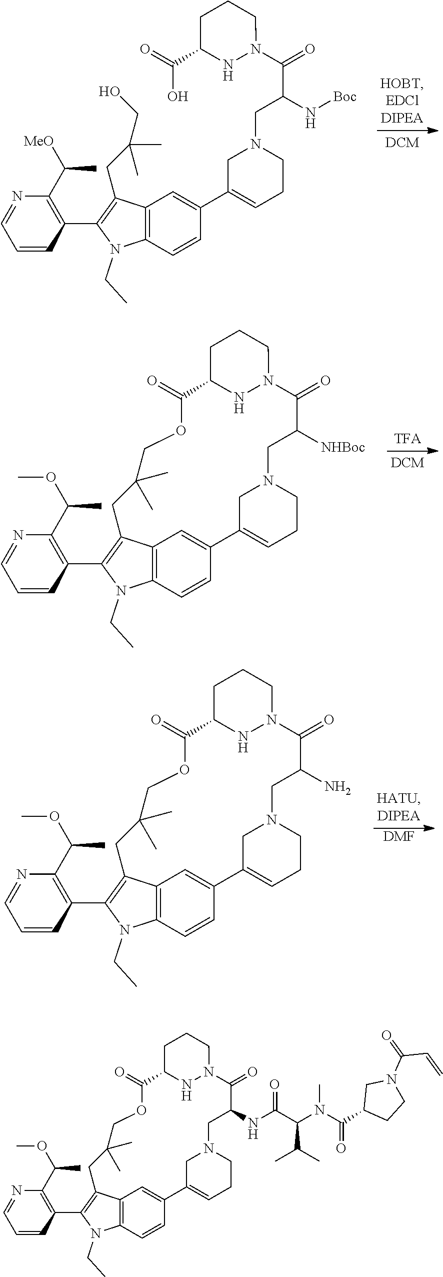

Step 1. To a solution of methyl (tert-butoxycarbonyl)-L-serinate (10 g, 45 mmol) in anhydrous MeCN (150 mL), was added DIPEA (17 g, 137 mmol). The reaction mixture was stirred at 45° C. for 2 h to give methyl 2-((tert-butoxycarbonyl)amino)acrylate in solution. LCMS (ESI): m/z: [M+Na] calc'd for C₉H₁₅NO₄ 201.1; found 224.1.

Step 2. To a solution of methyl 2-((tert-butoxycarbonyl)amino)acrylate (12 g, 60 mmol) in anhydrous MeCN (150 mL) at 0° C., was added 4-DMAP (13 g, 90 mmol) and (Boc)₂O (26 g, 120 mmol). The reaction was stirred for 6 h, then quenched with H₂O (100 mL) and extracted with DCM (200 mL×3). The combined organic layers were washed with brine (150 mL), dried over anhydrous sodium sulfate, filtered and concentrated under reduced pressure. The residue was purified by silica gel column chromatography to give methyl 2-(bis(tert-butoxycarbonyl)amino)acrylate (12.5 g, 65% yield) as solid. LCMS (ESI): m/z: [M+Na] calc'd for C₁₄H₂₃NO₆ 301.2; found 324.1.

Step 3. To a mixture of 5-bromo-1,2,3,6-tetrahydropyridine (8.0 g, 49 mmol) in MeOH (120 mL) under an atmosphere of Ar was added methyl 2-{bis[(tert-butoxy)carbonyl]amino}prop-2-enoate (22 g, 74 mmol). The mixture was stirred for 16 h, then concentrated under reduced pressure and the residue was purified by silica gel column chromatography to give methyl 2-(bis(tert-butoxycarbonyl)amino)-3-(5-bromo-3,6-dihydropyridin-1(2H)-yl)propanoate (12 g, 47% yield) as an oil. LCMS (ESI): m/z: [M+H] calc'd for C₁₉H₃₁BrN₂O₆ 462.1; found 463.1.

Step 4. To a mixture of methyl 2-(bis(tert-butoxycarbonyl)amino)-3-(5-bromo-3,6-dihydropyridin-1(2H)-yl)propanoate (14 g, 30 mmol) in 1,4-dioxane (30 mL) and H₂O (12 mL) was added LiOH (3.6 g, 151 mmol). The mixture was heated to 35° C. and stirred for 12 h, then 1M HCl was added and the pH adjusted to ˜3-4. The mixture was extracted with DCM (300 mL×2) and the combined organic layers were dried over anhydrous Na₂SO₄ and filtered. The filtrate was concentrated under reduced pressure to give 3-(5-bromo-3,6-dihydropyridin-1(2H)-yl)-2-((tert-butoxycarbonyl)amino)propanoic acid (10 g, 85% yield) as a solid. LCMS (ESI): m/z: [M+H] calc'd for C₁₃H₂₁BrN₂O₄ 348.1; found 349.0.

Step 5. To a mixture of 3-(5-bromo-3,6-dihydropyridin-1(2H)-yl)-2-((tert-butoxycarbonyl)amino)propanoic acid (10 g, 30 mmol), DIPEA (12 g, 93 mmol) and methyl (3S)-1,2-diazinane-3-carboxylate (5.4 g, 37 mmol) in DMF (100 mL) at 0° C. under an atmosphere of Ar was added HATU (13 g, 34 mmol). The mixture was stirred at 0° C. for 2 h, then H₂O added and the mixture extracted with EtOAc (300 mL×2). The combined organic layers were dried over anhydrous Na₂SO₄, filtered, the filtrate was concentrated under reduced pressure and the residue was purified by preparative-HPLC to give methyl (3S)-1-(3-(5-bromo-3,6-dihydropyridin-1(2H)-yl)-2-((tert-butoxycarbonyl)amino)propanoyl)hexahydropyridazine-3-carboxylate (9.0 g, 55% yield) as a solid. LCMS (ESI): m/z: [M+H] calc'd for C₁₉H₃₁BrN₄O₅ 474.1; found 475.1.

Step 6. A mixture of methyl (3S)-1-(3-(5-bromo-3,6-dihydropyridin-1(2H)-yl)-2-((tert-butoxycarbonyl)amino)propanoyl)hexahydropyridazine-3-carboxylate (9.0 g, 18 mmol), K₂CO₃ (4.5 g, 32 mmol), Pd(dppf)Cl₂·DCM (1.4 g, 2 mmol), 3-(1-ethyl-2-{2-[(1S)-1-methoxyethyl]pyridin-3-yl}-5-(4,4,5,5-tetramethyl-1,3,2-dioxaborolan-2-yl)indol-3-yl)-2,2-dimethylpropan-1-ol (9.8 g, 20 mmol) in 1,4-dioxane (90 mL) and H₂O (10 mL) under an atmosphere of Ar was heated to 75° C. and stirred for 2 h. H₂O was added and the mixture was extracted with EtOAc (200 mL×3). The combined organic layers were dried over Na₂SO₄, filtered, the filtrate was concentrated under reduced pressure and the residue was purified by silica gel column chromatography to give methyl (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylate (4.0 g, 25% yield) as a solid. LCMS (ESI): m/z [M+H] calc'd for C₄₂H₆₀N₆O₇ 760.5; found 761.4.

Step 7. To a mixture of methyl (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylate (4.1 g, 5.0 mmol) in THF (35 mL) at 0° C. was added LiOH (0.60 g, 27 mmol). The mixture was stirred at 0° C. for 1.5 h, then 1M HCl added to adjust pH to ˜6-7 and the mixture extracted with EtOAc (200 mL×3). The combined organic layers were dried over Na₂SO₄, filtered and the filtrate was concentrated under reduced pressure to give (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylic acid (3.6 g, 80% yield) as a solid. LCMS (ESI): m/z: [M+H] calc'd for C₄₁H₅₈N₆O₇ 746.4; found 747.4.

Step 8. To a mixture of (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylic acid (3.6 g, 5.0 mmol) and DIPEA (24 g,190 mmol) in DCM (700 mL) under an atmosphere of Ar was added EDCl·HCl (28 g, 140 mmol) and HOBT (6.5 g, 50 mmol). The mixture was heated to 30° C. and stirred for 16 h at 30° C., then concentrated under reduced pressure. The residue was diluted with EtOAc (200 mL) and washed with H₂O (200 mL×2), brine (200 mL), dried over Na₂SO₄ and filtered. The filtrate was concentrated under reduced pressure and the residue was purified by silica gel column chromatography to give tert-butyl ((6³S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (1.45 g, 40% yield) as a solid. LCMS (ESI): m/z: [M+H] calc'd for C₄₁H₅₆N₆O₆ 728.4; found 729.4.

Step 9. To a mixture of tert-butyl ((6³S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (130 mg, 0.20 mmol) in DCM (1.0 mL) at 0° C. was added TFA (0.3 mL). The mixture was warmed to room temperature and stirred for 2 h, then concentrated under reduced pressure to give (6³S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione, which was used directly in the next step directly without further purification. LCMS (ESI): m/z: [M+H] calc'd for C₃₆H₄₈N₆O₄ 628.4; found 629.4.

Step 10. To a mixture of ((6³S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (130 mg, 0.2 mmol), DIPEA (270 mg, 2.0 mmol) and (2S)-3-methyl-2-{N-methyl-1-[(3S)-1-(prop-2-enoyl)pyrrolidin-3-yl]formamido}butanoic acid (118 mg, 0.40 mmol) in DMF (3.0 mL) at 0° C. under an atmosphere of Ar was added HATU (87 mg, 0.30 mmol) in portions. The mixture was stirred at 0° C. for 1 h, then diluted with H₂O extracted with EtOAc (30 mL×2). The combined organic layers were dried over anhydrous Na₂SO₄, filtered, the filtrate was concentrated under reduced pressure and the residue was purified by preparative-HPLC to give (3S)-1-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N-methylpyrrolidine-3-carboxamide (17.2 mg, 10% yield) as a solid. LCMS (ESI): m/z: [M+H] calc'd for C₅₀H₆₈N₆O₄ 892.5; found 893.5; ¹H NMR (400 MHz, CD₃OD) δ 8.74 (d, J=4.4 Hz, 1H), 7.93-7.90 (m, 1H), 7.56-7.51 (m, 3H), 7.43 (d, J=4.4 Hz, 1H), 6.63-6.53 (m, 2H), 6.33-6.23 (m, 2H), 5.83-5.70 (m, 1H), 4.73-4.70 (d, J=11.0 Hz, 1H), 4.48-4.45 (d, J=13.0 Hz, 1H), 4.12-4.10 (m, 3H), 3.86-3.81 (m, 4H), 3.79-3.75 (m, 1H), 3.72-3.69 (m, 3H), 3.57-3.47 (m, 2H), 3.21-3.09 (m, 1H), 3.07-3.04 (q, 4H), 3.02-2.95 (m, 3H), 2.86-2.82 (m, 3H), 2.66-2.48 (m, 2H), 2.29-2.17 (m, 4H), 2.11-1.98 (m, 2H), 1.95-1.91 (m, 1H), 1.45 (d, J=6.2 Hz, 3H), 1.23-1.16 (m, 2H), 1.09-1.04 (m, 1H), 0.97-0.93 (m, 3H), 0.92-0.81 (m, 5H), 0.67-0.63 (m, 3H).

### Example A663. The synthesis of (2R)-2-(((1-(4-(dimethylamino)-4-methylpent-2-ynoyl)870yridine870-3-yl)oxy)methyl)-N-((6³S,4S)-1¹-ethyl-1²-(2-((S)-1-methoxyethyl)870yridine-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)- pyridazina-2(5,1)-pyridinacycloundecaphane-4-yl)-3-methylbutanamide

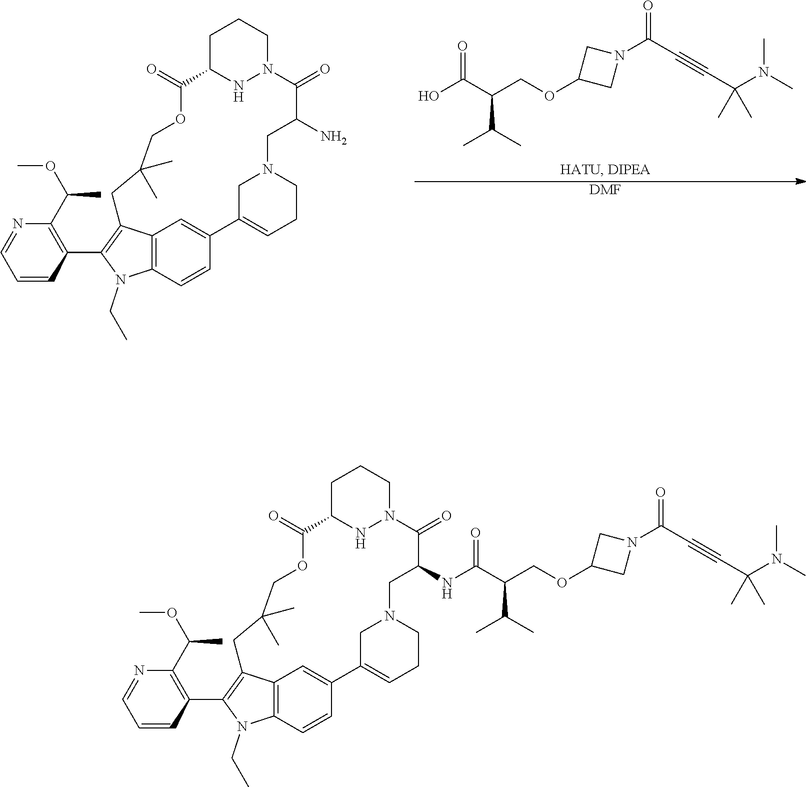

To a mixture of (6³S,4S)-4-amino-1¹-ethyl-1²-(2-((S)-1-methoxyethyl)870yridine-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (100 mg, 0.16 mmol),®-2-(((1-(4-(dimethylamino)-4-methylpent-2-ynoyl)870yridine870-3-yl)oxy)methyl)-3-methylbutanoic acid (80 mg, 0.24 mmol) and DIPEA (825 mg, 6.4 mmol) in DMF (2 mL) at 0° C., was added HATU (95 mg, 0.24 mmol). The reaction mixture was stirred at 0° C. for 1 h, then poured into H₂O (60 mL), extracted with EtOAc (80 mL×2). The combined organic layers were washed with H₂O (80 mL) and brine (80 mL), dried over anhydrous sodium sulfate, filtered and concentrated under reduced pressure. The residue was purified by silica gel chromatography to afford (2R)-2-(((1-(4-(dimethylamino)-4-methylpent-2-ynoyl)870yridine870-3-yl)oxy)methyl)-N-((6³S,4S)-1¹-ethyl-1²-(2-((S)-1-methoxyethyl)870yridine-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)-3-methylbutanamide (55 mg, 36% yield) as solid. ¹H NMR (400 MHz, CD₃OD) δ 8.76-8.70 (m, 1H), 8.49 (dd, J=4.3, 1.4 Hz, 0.1H), 7.93-7.87 (m, 1H), 7.58-7.50 (m, 3H), 7.41 (dd, J=8.8, 3.2 Hz, 1H), 6.26 (d, J=16.8 Hz, 1H), 5.96 (t, J=9.6 Hz, 1H), 4.47 (d, J=12.8 Hz, 1H), 4.39-4.28 (m, 2H), 4.21-3.97 (m, 5H), 3.96-3.70 (m, 5H), 3.68-3.54 (m, 3H), 3.51-3.35 (m, 1H), 3.11 (d, J=22.7 Hz, 3H), 3.00-2.67 (m, 5H), 2.46-2.30 (m, 7H), 2.24 (s, 3H), 2.11 (d, J=12.4 Hz, 1H), 1.92 (d, J=13.2 Hz, 1H), 1.85-1.60 (m, 3H), 1.45 (d, J=7.8 Hz, 6H), 1.32 (d, J=16.0 Hz, 3H), 1.12 (dt, J=24.5, 6.8 Hz, 3H), 0.95 (m, 6H), 0.76 (m, 6H). LCMS (ESI): m/z: [M+H] calc'd for C₅₃H₇₄N₈O₇ 934.6; found 935.5.

### Example A646. The synthesis of (2R)-2-(((1-(4-(dimethylamino)-4-methylpent-2-ynoyl)azetidin-3-yl)oxy)methyl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)-3-methylbutanamide

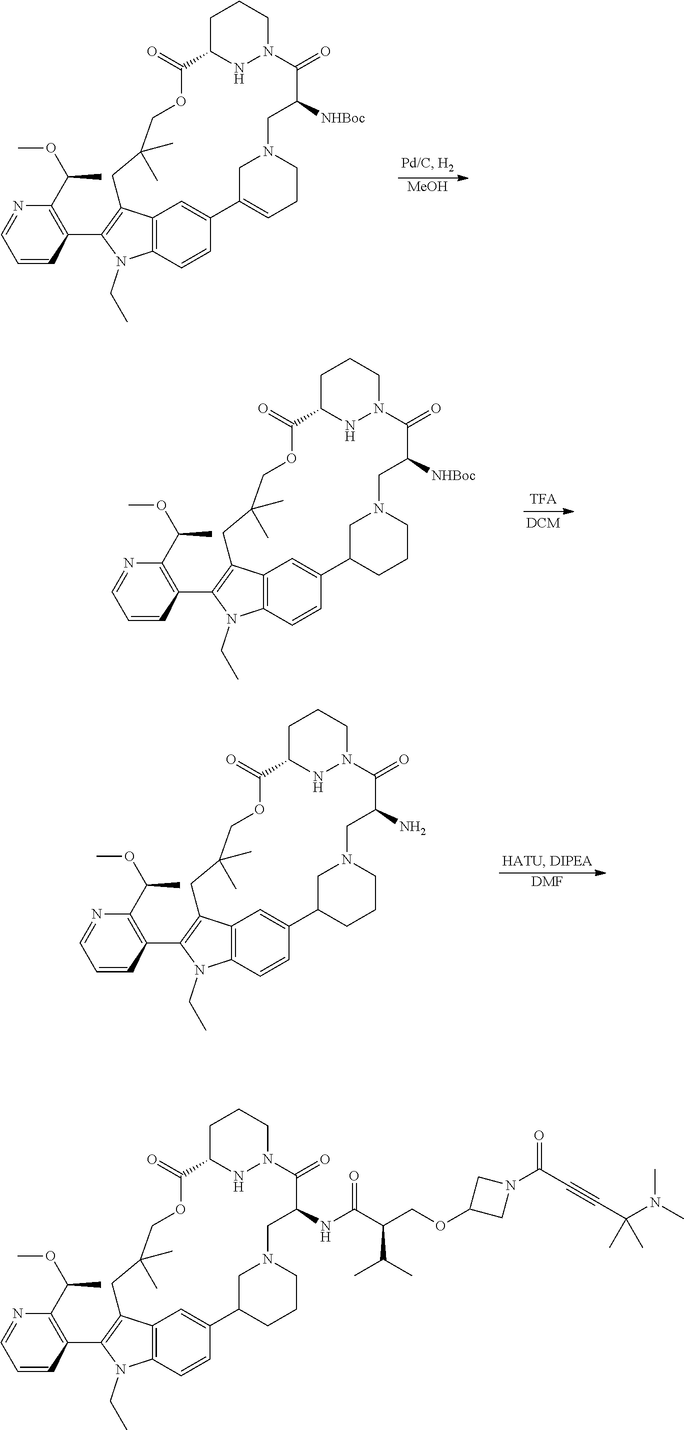

Step 1. A mixture of tert-butyl ((6³S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (0.2 g, 0.28 mmol) and Pd/C (0.2 g, 2 mmol) in MeOH (10 mL) was stirred at 25° C. for 16 h under an H₂ atmosphere. The reaction mixture was filtered through Celite, concentrated under reduced pressure to afford tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate as solid. LCMS (ESI): m/z: [M+H] calc'd for C₄₁H₅₈N₆O₆ 730.4; found 731.4.

Step 2. To a solution of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate (150 mg, 0.2 mmol) in DCM (1.5 mL) at 0° C. was added TFA (0.5 mL). The reaction mixture was stirred at 20° C. for 1 h, then concentrated under reduced pressure to afford (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione as solid. LCMS (ESI): m/z: [M+H] calc'd for C₃₆H₅₀N₆O₄ 630.4; found 631.4.

Step 3. To a mixture of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (240 mg, 0.4 mmol), DIPEA (982 mg, 2 mmol) and (R)-2-(((1-(4-(dimethylamino)-4-methylpent-2-ynoyl)azetidin-3-yl)oxy)methyl)-3-methylbutanoic acid (148 mg, 0.45 mmol) in DMF (4 mL) at 0° C. under argon atmosphere, was added HATU (173 mg, 0.46 mmol) in portions. The reaction mixture was stirred at 0° C. under an argon atmosphere for 1 h, then quenched with H₂O at 0° C. The resulting mixture was extracted with EtOAc (30 mL×2). The combined organic layers were dried over anhydrous sodium sulfate, filtered and concentrated under reduced pressure. The residue was purified by reverse phase chromatography to afford (2R)-2-(((1-(4-(dimethylamino)-4-methylpent-2-ynoyl)azetidin-3-yl)oxy)methyl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)-3-methylbutanamide (150 mg, 38% yield) as solid. ¹H NMR (400 MHz, CD3OD) δ 8.72 (d, J=4.8 Hz, 1H), 7.88 (d, J=8.0 Hz, 1H), 7.53-7.49 (m, 2H), 7.42 (d, J=8.4 Hz, 1H), 7.18 (d, J=8.4 Hz, 1H), 5.95-5.91 (m, 1H), 4.52-4.49 (m, 1H), 4.37-4.25 (m, 3H), 4.18-4.15 (m, 2H), 3.99-3.98 (m, 2H), 3.90-3.86 (m, 1H), 3.76-3.68 (m, 2H), 3.55-3.50 (m, 2H), 3.39-3.36 (m, 2H), 3.20 (s, 3H), 3.02 (s, 3H), 2.89-2.79 (m, 3H), 2.62-2.50 (m, 2H), 2.36 (s, 3H), 2.35-2.30 (m, 1H), 2.26 (s, 3H), 2.20-1.15 (m, 1H), 1.97-1.93 (m, 3H), 1.81-1.76 (m, 4H), 1.64-1.61 (m, 2H), 1.46-1.43 (m, 6H), 1.36 (d, J=14.8 Hz, 3H), 1.02 (s, 3H), 0.94 (m, 6H), 0.81 (s, 3H), 0.65 (s, 3H). LCMS (ESI): m/z: [M+H] calc'd for C₅₃H₇₆N₈O₇ 936.6; found 937.5.

### Example A740. Synthesis of (3S)-1-acryloyl-N-((2S)-1-(((2³S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylpyrrolidine-3-carboxamide

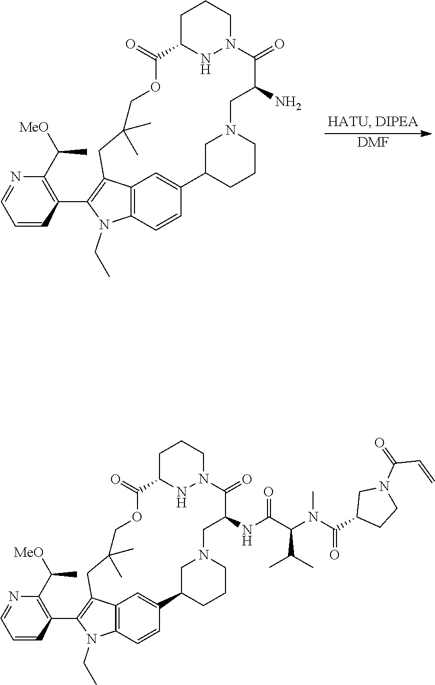

To a mixture of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (140 mg, 0.20 mmol), DIPEA (570 mg, 4.4 mmol) and (2S)-3-methyl-2-{N-methyl-1-[(3S)-1-(prop-2-enoyl)pyrrolidin-3-yl]formamido}butanoic acid (124 mg, 0.40 mmol) in DMF (3.0 mL) at 0° C. under an atmosphere of Ar was added HATU (100 mg, 0.30 mmol) in portions. The mixture was stirred at 0° C. for 1 h, then H₂O was added and the mixture extracted with EtOAc (2×30 mL). The combined organic layers were dried over anhydrous Na₂SO₄ and filtered. The filtrate was concentrated under reduced pressure and the residue was purified by preparative-HPLC to give (3S)-1-acryloyl-N-((2S)-1-(((2³S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylpyrrolidine-3-carboxamide (41 mg, 20% yield) as a solid. LCMS (ESI): m/z: [M+H] calc'd for C₅₀H₇₀N₆O₇ 894.5; found 895.5; ¹H NMR (400 MHz, CD₃OD) δ 8.72 (d, J=4.8 Hz, 1H), 7.87 (d, J=7.6 Hz, 1H), 7.51-7.49 (m, 2H), 7.42-7.37 (m, 1H), 7.18-7.14 (m, 1H), 6.64-6.54 (m, 1H), 6.30-6.23 (m, 1H), 5.77-5.70 (m, 2H), 4.65-4.60 (m, 1H), 4.50-4.40 (m, 1H), 4.27-4.16 (m, 2H), 4.00-3.95 (m, 2H), 3.83-3.78 (m, 2H), 3.73-3.60 (m, 4H), 3.51-3.36 (m, 3H), 3.22-3.19 (m, 4H), 3.07 (d, J=6.8 Hz, 2H), 2.99 (d, J=12.0 Hz, 3H), 2.90-2.78 (m, 2H), 2.75-2.64 (m, 3H), 2.20-2.10 (m, 4H), 2.02-1.93 (m, 3H), 1.87-1.64 (m, 4H), 1.45 (d, J=4.8 Hz, 3H), 1.06-1.00 (m, 4H), 0.97-0.89 (m, 3H), 0.83-0.79 (m, 3H), 0.66 (s, 3H).

### Example A534. (2S)-2-((S)-7-(4-(dimethylamino)-4-methylpent-2-ynoyl)-1-oxo-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

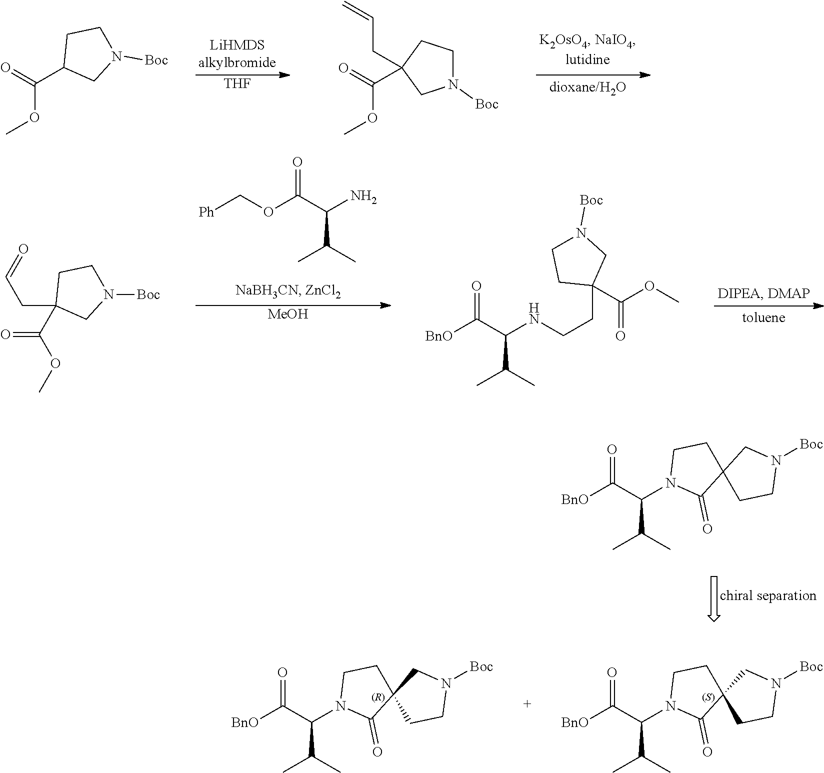

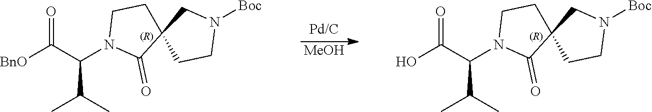

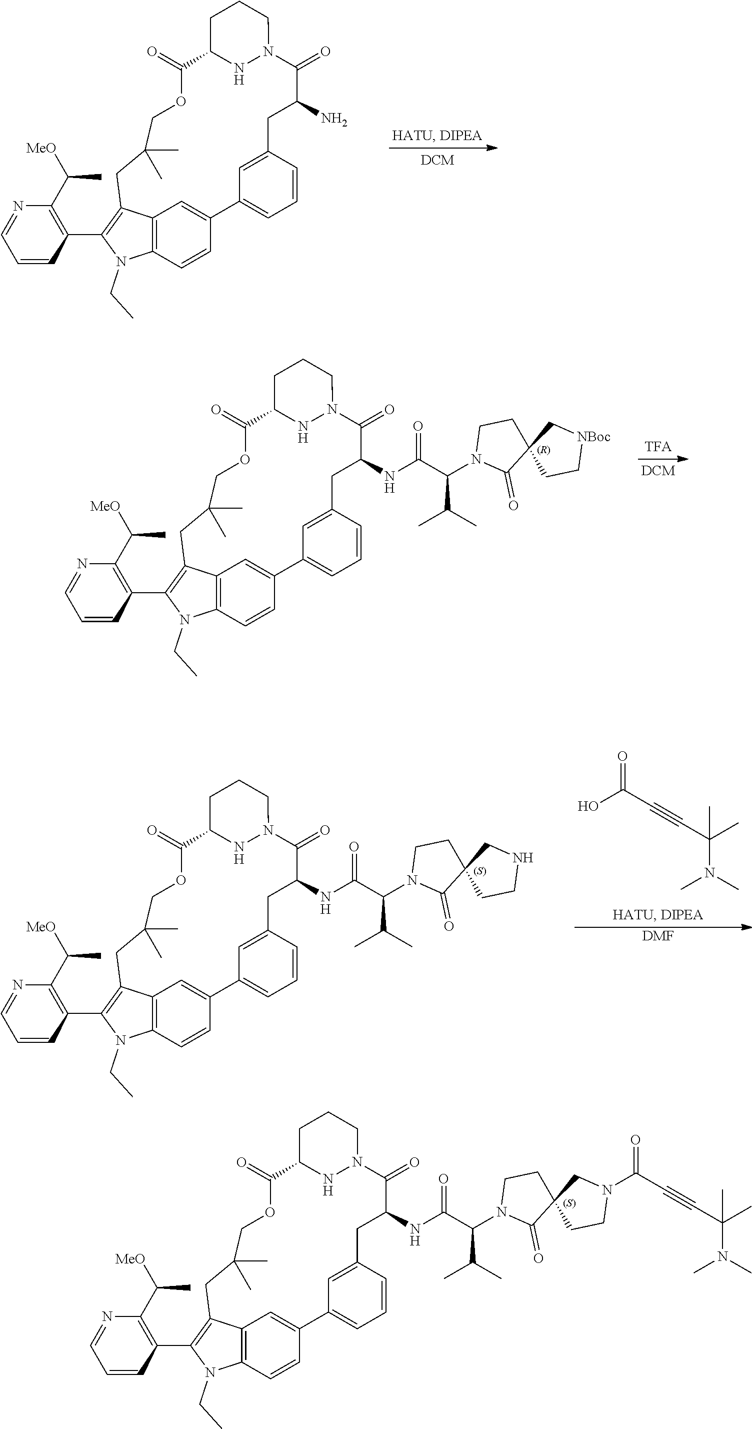

Step 1. To a mixture of 1-tert-butyl 3-methyl pyrrolidine-1,3-dicarboxylate (20.0 g, 87.2 mmol) in THF (150 mL) at −78° C. under an atmosphere of nitrogen was added 1M LiHMDS in THF (113.4 mL, 113.4 mmol). After stirring at −78° C. for 40 min, allyl bromide (13.72 g, 113.4 mmol) was added and the mixture was allowed to warm to room temperature and stirred for 4 h. The mixture was cooled to 0° C., saturated NaCl (30 mL) was added and the mixture extracted with EtOAc. The combined organic layers were dried over anhydrous Na₂SO₄ and filtered. The filtrate was concentrated under reduced pressure and the residue was purified by silica gel column chromatography to give 1-(tert-butyl) 3-methyl 3-allylpyrrolidine-1,3-dicarboxylate (17 g, 72% yield) as an oil, ¹H NMR (300 MHz, CDCl₃) δ 5.80-5.60 (m, 1H), 5.16-5.02 (m, 2H), 3.71 (s, 4H), 3.42 (d, J=9.3 Hz, 2H), 3,27 (t, J=11.2 Hz, 1H), 2,42 (d, J=7.6 Hz, 2H), 2.38-2.24 (m, 1H), 2.05 (s, 1H), 1.85 (dt, J=14.3, 7.5 Hz, 1H), 1.46 (s, 10H), 1.27 (t, J=7.1 Hz, 1H).

Step 2. To a mixture of 1-(tertbutyl) 3-methyl 3-allylpyrrolidine-1,3-dicarboxylate (4.0 g, 14.9 mmol) and 2,6-dimethylpyridine (3.18 g, 29.7 mmol) in 1,4-dioxane (200 mL) and H₂O (100 mL) at 0° C. was added K₂OsO₄ 2H₂O (0.11 g, 0.3 mmol) in portions. The mixture was stirred for 15 min at 0° C., then NaIO₄ (6.35 g, 29.7 mmol) was added in portions. The mixture was stirred at room temperature for 3 h at room temperature, then cooled to 0° C. and saturated aqueous Na₂S₂O₃ (50 mL) added. The mixture was extracted with EtOAc (3×100 mL) and the combined organic layers were washed with 2 M HCl, then dried over anhydrous Na₂SO₄ and filtered. The filtrate was concentrated under reduced pressure to give 1-(tert-butyl) 3-methyl 3-(2-oxoethyl)pyrrolidine-1,3-dicarboxylate (4 g, 52% yield) as an oil. ¹H NMR (300 MHz, CDCl₃) δ 5.80-5.60 (m, 1H), 5.16-5.04 (m, 2H), 3.72 (s, 3H), 3.41 (s, 3H), 3.28 (d, J=11.0 Hz, 1H), 2.44 (s, 2H), 2.31 (d, J=9.1 Hz, 1H), 1.85 (dt, J=12.7, 7.5 Hz, 1H), 1.69 (s, 1H), 1.47 (s, 10H).

Step 3. To a mixture of 1-(tert-butyl) 3-methyl 3-(2-oxoethyl)pyrrolidine-1,3-dicarboxylate (6.30 g, 23.2 mmol), in MeOH (70 mL) at 0° C. was added benzyl (2S)-2-amino-3-methylbutanoate (7.22 g, 34.8 mmol) and ZnCl₂ (4.75 g, 34.8 mmol). The mixture was warmed to room temperature and stirred for 30 min, then cooled to 0° C. and NaCNBH₃ (2.92 g, 46.4 mmol) was added in portions. The mixture was warmed to room temperature and stirred for 2 h, then cooled to 0° C. and saturated aqueous NH₄Cl added. The mixture was extracted with EtOAc (3×200 mL) and the combined organic layers were washed with brine (150 mL), dried over anhydrous Na₂SO₄ and filtered. The filtrate was concentrated under reduced pressure and the residue was purified by silica gel column chromatography to give 1-(tert-butyl) 3-methyl 3-(2-(((S)-1-(benzyloxy)-3-methyl-1-oxobutan-2-yl)amino)ethyl)pyrrolidine-1,3-dicarboxylate (6.4 g, 54% yield) as an oil. LCMS (ESI): m/z: [M+H] calc'd for C₂₅H₃₈N₂O₆ 462.3; found 463.4.

Step 4. To a mixture of 1-(tert-butyl) 3-methyl 3-(2-(((S)-1-(benzyloxy)-3-methyl-1-oxobutan-2-yl)amino)ethyl)pyrrolidine-1,3-dicarboxylate (4.50 g, 9.7 mmol) in toluene (50 mL) was added DIPEA (12.57 g, 97.3 mmol) and DMAP (1.19 g, 9.7 mmol). The resulting mixture was heated to 80° C. and stirred for 24 h, then concentrated under reduced pressure and the residue was purified by preparative-HPLC, then by chiral-HPLC to give tert-butyl (R)-7-((S)-1-(benzyloxy)-3-methyl-1-oxobutan-2-yl)-6-oxo-2,7-diazaspiro[4.4]nonane-2-carboxylate (1.0 g, 32% yield) and tert-butyl (S)-7-((S)-1-(benzyloxy)-3-methyl-1-oxobutan-2-yl)-6-oxo-2,7-diazaspiro[4.4]nonane-2-carboxylate (1.0 g, 32% yield) and as a an oil. LCMS (ESI): m/z: [M+H] calc'd for C₂₄H₃₄N₂O₅ 430.5; found 431.2 and LCMS (ESI): m/z: [M+H] calc'd for C₂₄H₃₄N₂O₅ 430.3; found 431.2.

Step 5. A mixture of tert-butyl (R)-7-((S)-1-(benzyloxy)-3-methyl-1-oxobutan-2-yl)-6-oxo-2,7-diazaspiro[4.4]nonane-2-carboxylate (4.0 g) and 10% Pd/C (1 g) in MeOH (40 mL) was stirred at room temperature under an atmosphere of H₂. The mixture was filtered through a pad of Celite pad and the filtrate was concentrated under reduced pressure to give (S)-2-((R)-7-(tert-butoxycarbonyl)-1-oxo-2,7-diazaspiro[4.4]nonan-2-yl)-3-methylbutanoic acid (4.9 g) as a solid. LCMS (ESI): m/z: [M−H] calc'd for C₁₇H₂₈N₂O₅ 340.2; found 339.3.

Step 6. To a mixture of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (500 mg, 0.8 mmol) in DCM at 0° C. were added DIPEA (829 mg, 6.4 mmol), ((S)-2-((R)-7-(tert-butoxycarbonyl)-1-oxo-2,7-diazaspiro[4.4]nonan-2-yl)-3-methylbutanoic acid (273 mg, 0.8 mmol) and HATU (396 mg, 1.0 mmol) in portions over 1 min. The mixture was allowed to warm to room temperature and stirred 2 h, then concentrated under reduced pressure and the residue was purified by preparative-TLC to give tert-butyl (5R)-7-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-6-oxo-2,7-diazaspiro[4.4]nonane-2-carboxylate (500 mg, 64% yield) as an oil. LCMS (ESI): m/z: [M+H] calc'd for C₅₄H₇₁N₇O₈ 945.5; found 946.5.

Step 7. To a mixture of tert-butyl (5R)-7-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-6-oxo-2,7-diazaspiro[4.4]nonane-2-carboxylate (1.0 g, 1.06 mmol) in DCM (10 mL) at 0° C. was added TFA (3 mL) dropwise. The mixture was warmed to room temperature and stirred for 1 h, then concentrated under reduced pressure to give (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-1-oxo-2,7-diazaspiro[4.4]nonan-2-yl)butanamide (1.3 g). LCMS (ESI): m/z: [M−H] calc'd for C₄₉H₆₃N₇O₆ 846.1; found 845.5.

Step 8. To a mixture of (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-1-oxo-2,7-diazaspiro[4.4]nonan-2-yl)butanamide (500 mg, 0.59 mmol) and DIPEA (764 mg, 5.9 mmol) in DMF (5 mL) at 0° C. were added 4-(dimethylamino)-4-methylpent-2-ynoic acid (110 mg, 0.71 mmol) and HATU (292 mg, 0.77 mmol) in portions. The mixture was warmed to room temperature and stirred for 1 h, then H₂O (10 mL) was added and the mixture extracted with EtOAc (10 mL×3). The combined organic layers were washed with brine (10 mL), dried over anhydrous Na₂SO₄ and filtered. The filtrate was concentrated under reduced pressure and the residue was purified by preparative-HPLC to give (2S)-2-((S)-7-(4-(dimethylamino)-4-methylpent-2-ynoyl)-1-oxo-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (177 mg, 28.94% yield) as a white solid. LCMS (ESI): m/z: [M+H] calc'd for C₅₇H₇₄N₈O₇ 982.6; found 983.8; ¹H NMR (400 MHz, DMSO-d₆) δ 8.76 (dd, J=4.7, 1.7 Hz, 1H), 8.52 (d, J=7.9 Hz, 1H), 7.99 (d, J=1.7 Hz, 1H), 7.83 (d, J=10.2 Hz, 2H), 7.74-7.58 (m, 3H), 7.53 (dd, J=7.7, 4.8 Hz, 1H), 7.24 (t, J=7.6 Hz, 1H), 7.12 (d, J=7.6 Hz, 1H), 5.32 (d, J=9.7 Hz, 2H), 4.33-4.20 (m, 4H), 4.03 (dd, J=15.0, 8.6 Hz, 2H), 3.88-3.82 (m, 1H), 3.63 (dq, J=20.5, 10.3 Hz, 4H), 3.42-3.34 (m, 2H), 3.21 (s, 1H), 3.13 (d, J=2.8 Hz, 3H), 2.87 (s, 2H), 2.83-2.72 (m, 2H), 2.69-2.62 (m, 1H), 2.21 (d, J=22.6 Hz, 6H), 2.12-1.76 (m, 7H), 1.75-1.47 (m, 2H), 1.46-1.28 (m, 9H), 0.99-0.89 (m, 6H), 0.79-0.71 (m, 6H), 0.52 (s, 3H).

### Example A341. Synthesis of (3S)-1-acryloyl-N-((2S)-1-(((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide

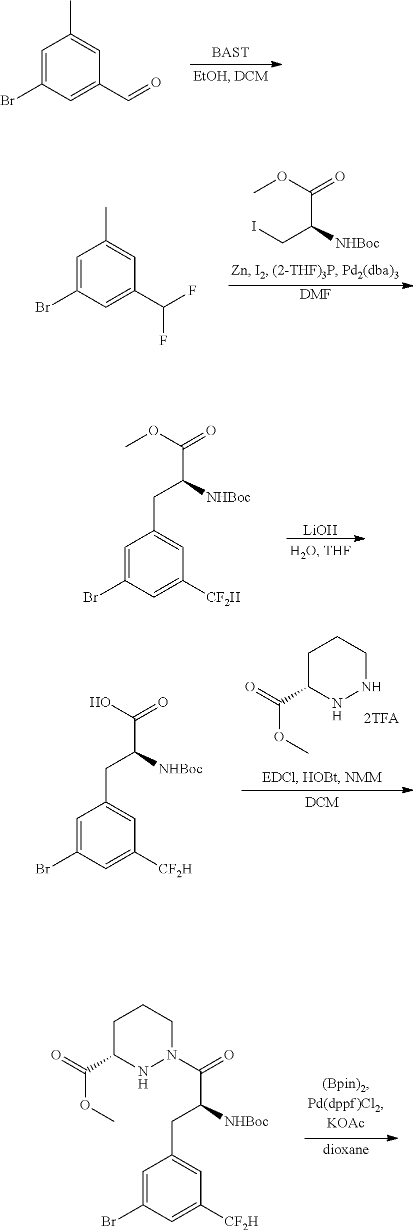

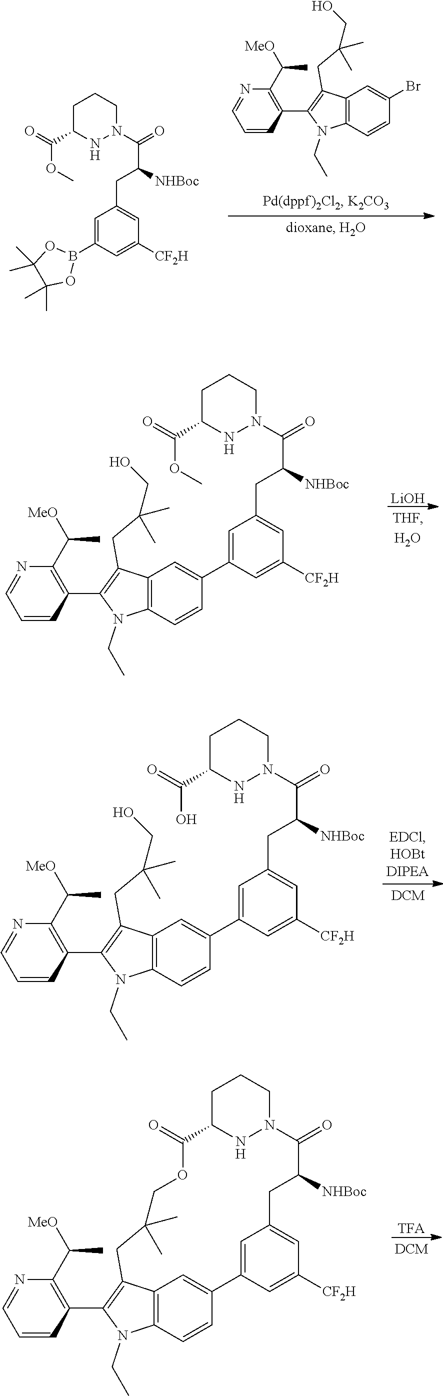

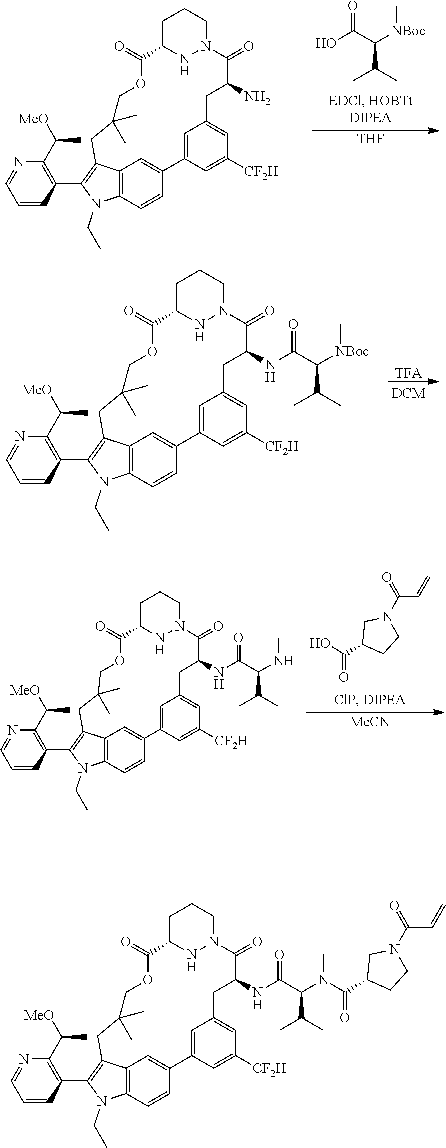

Step 1. To a mixture of 3-bromo-5-iodobenzaldehyde (4.34 g, 14.0 mmol) in DCM at 0° C. under an atmosphere of N₂ was added BAST (6.8 g, 30.7 mmol) and EtOH (129 mg, 2.8 mmol) dropwise. The mixture was heated with microwave heating at 27° C. for 14 h. H₂O (500 mL) was added and the mixture was extracted with DCM (200 mL×3), the combined organic layers were concentrated under reduced pressure and the residue was purified by silica gel column chromatography to give 1-bromo-3-(difluoromethyl)-5-iodobenzene (3.2 g, 65% yield) as a solid. ¹H NMR (300 MHz, DMSO-d₆) δ 8.16 (p, J=1.2 Hz, 1H), 7.94 (p, J=1.3 Hz, 1H), 7.81 (p, J=1.3 Hz, 1H), 7.00 (t, J=55.3 Hz, 1H).

Step 2. A mixture of Zn (2.28 g, 34.8 mmol) and 12 (442 mg, 1.74 mmol) in DMF (20 mL) under an atmosphere of Ar was stirred at 50° C. for 0.5 h. To this mixture was added a solution of methyl (methyl (R)-2-((tert-butoxycarbonyl)amino)-3-iodopropanoate (2.39 g, 7.25 mmol) in DMF (20 mL) and the mixture was stirred at 50° C. for 2 h. After cooling, the mixture was added to 1-bromo-3-(difluoromethyl)-5-iodobenzene (2.90 g, 8.7 mmol), Pd₂(dba)₃ (239 mg, 0.26 mmol) and tri-2-furylphosphine (162 mg, 0.7 mmol) in DMF (20 mL). The mixture was heated to 70° C. and stirred for 2 h, then H₂O (200 mL) was added and the mixture extracted with EtOAc (200 mL×3). The combined organic layers were concentrated under reduced pressure and the residue was purified by silica gel column chromatography to give methyl (S)-3-(3-bromo-5-(difluoromethyl)phenyl)-2-((tert-butoxycarbonyl)amino)propanoate (560 mg, 19% yield) as a solid. ¹H NMR (300 MHz, DMSO-d₆) δ 7.65 (d, J=10.0 Hz, 2H), 7.47 (s, 1H), 7.36 (d, J=8.4 Hz, 1H), 7.00 (t, J=55.6 Hz, 1H), 4.25 (td, J=9.6, 4.7 Hz, 1H), 3.64 (s, 3H), 3.11 (dd, J=13.6, 4.9 Hz, 1H), 3.00-2.80 (m, 1H), 1.32 (s, 9H).

Step 3. To a mixture of methyl (S)-3-(3-bromo-5-(difluoromethyl)phenyl)-2-((tert-butoxycarbonyl)amino)propanoate (650 mg, 1.6 mmol) in THF (1.5 mL) at 0° C. under an atmosphere of N₂ was added LiOH (114 mg, 4.8 mmol) in H₂O (1.50 mL). The mixture was stirred at 0° C. for 1 h, then acidified to pH 5 with 1M HCl. The mixture was extracted with DCM/MeOH (10/1) (100 mL×3) and the combined organic layers were dried over anhydrous Na₂SO₄ and filtered. The filtrate was concentrated under reduced pressure to give (S)-3-(3-bromo-5-(difluoromethyl)phenyl)-2-((tert-butoxycarbonyl)amino)propanoic acid (500 mg), which was used directly in the next step without further purification. LCMS (ESI): m/z: [M+H] calc'd for C₁₅H₁₈BrF₂NO₄ 393.0; found 392.1.

Step 4. To a mixture of methyl (3S)-1,2-diazinane-3-carboxylate (475 mg, 3.3 mmol) in DCM (10 mL) at 0° C. under an atmosphere of N₂ were added N-methylmorpholine (3.34 g, 33.0 mmol) and (S)-3-(3-bromo-5-(difluoromethyl)phenyl)-2-((tert-butoxycarbonyl)amino)propanoic acid (650 mg, 1.7 mmol) and HOBt (45 mg, 0.33 mmol) and EDCI (632 mg, 3.3 mmol). The mixture was warmed to room temperature and stirred for 16 h, then diluted with DCM (100 mL) and H₂O. The organic and aqueous layer was separated and the aqueous layer was extracted with DCM (100 mL×3). The combined organic layers were dried over anhydrous Na₂SO₄ and filtered. The filtrate was concentrated under reduced pressure and the residue was purified by silica gel column chromatography to give methyl (S)-1-((S)-3-(3-bromo-5-(difluoromethyl)phenyl)-2-((tert-butoxycarbonyl)amino)propanoyl)hexahydropyridazine-3-carboxylate (510 mg, 56% yield) as a solid. LCMS (ESI): m/z: [M+H] calc'd for C₂₁H₂₈BrF₂N₃O₅ 519.1; found 520.3.

Step 5. To a mixture of 4,4,5,5-tetramethyl-2-(tetramethyl-1,3,2-dioxaborolan-2-yl)-1,3,2-dioxaborolane (488 mg, 1.92 mmol) and methyl (S)-1-((S)-3-(3-bromo-5-(difluoromethyl)phenyl)-2-((tert-butoxycarbonyl)amino)propanoyl)hexahydropyridazine-3-carboxylate (500 mg, 0.96 mmol) in 1,4-dioxane (5 mL) was added Pd(dppf)Cl₂ (70 mg, 0.07 mmol) and KOAc (236 mg, 2.4 mmol) in portions. The mixture was heated to 90° C. and stirred for 4 h then diluted with H₂O (100 mL). The mixture was extracted with DCM (100 mL×3) and the combined organic layers were dried over anhydrous Na₂SO₄ and filtered. The filtrate was concentrated under reduced pressure and the residue was purified by silica gel column chromatography to give methyl (S)-1-((S)-2-((tert-butoxycarbonyl)amino)-3-(3-(difluoromethyl)-5-(4,4,5,5-tetramethyl-1,3,2-dioxaborolan-2-yl)phenyl)propanoyl)hexahydropyridazine-3-carboxylate (423 mg, 73% yield) as a solid. LCMS (ESI): m/z: [M+H] calc'd for C₂₇H₄₀BF₂N₃O₇ 567.3; found 568.2.

Step 6. To a mixture of methyl (S)-1-((S)-2-((tert-butoxycarbonyl)amino)-3-(3-(difluoromethyl)-5-(4,4,5,5-tetramethyl-1,3,2-dioxaborolan-2-yl)phenyl)propanoyl)hexahydropyridazine-3-carboxylate (260 mg, 0.47 mmol), (S)-3-(5-bromo-1-ethyl-2-(2-(1-methoxyethyl)pyridin-3-yl)-1H-indol-3-yl)-2,2-dimethylpropan-1-ol and Pd(dppf)Cl₂ (34 mg, 0.05 mmol) in 1,4-dioxane (3 mL) and H₂O (0.6 mL) was added K₂CO₃ (163 mg, 1.12 mmol). The mixture was heated to 60° C. and stirred for 16 h, then diluted with H₂O (100 mL) and extracted with DCM (100 mL×3). The combined organic layers were dried over anhydrous Na₂SO₄ and filtered. The filtrate was concentrated under reduced pressure and the residue was purified by silica gel column chromatography to give methyl (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylate (350 mg, 78% yield) as a solid. LCMS (ESI): m/z: [M+H] calc'd for C₄₄H₅₇F₂N₅O₇ 805.4; found 806.6.

Step 7. To a mixture of methyl (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylate (350 mg, 0.43 mmol) in THF (2.8 mL) at 0° C. was added LiOH H₂O (54 mg, 1.3 mmol) in H₂O (0.7 mL). The mixture was warmed to room temperature and stirred for 2 h, then acidified to pH 5 with 1M HCl and extracted with EtOAc (50 mL×3). The combined organic layers were dried over anhydrous Na₂SO₄, filtered and the filtrate was concentrated under reduced pressure to give (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylic acid (356 mg) was used directly in the next step without further purification. LCMS (ESI): m/z: [M+H] calc'd for C₄₃H₅₅F₂N₅O₇ 791.4; found 792.6.

Step 8. To a mixture of S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylic acid (356 mg, 0.45 mmol) and DIPEA (1.74 g, 13.5 mmol) in DCM were added EDCI (2.41 g, 12.6 mmol) and HOBt (304 mg, 2.3 mmol). The mixture was stirred for 16 h then H₂O was added and the mixture extracted with EtOAc (200 mL×3). The combined organic layers were washed with brine (50 mL×4), dried over anhydrous Na₂SO₄ and filtered. The filtrate was concentrated under reduced pressure and the residue was purified by silica gel column chromatography to give tert-butyl ((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (202 mg, 51% yield). LCMS (ESI): m/z: [M+H] calc'd for C₄₃H₅₃F₂N₅O₆ 773.4; found 774.6.

Step 9. To a mixture of tert-butyl ((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (202 mg, 0.26 mmol) in DCM (2 mL) at 0° C. was added TFA (1.0 mL) dropwise. The mixture was stirred at 0° C. for 1.5 h, then concentrated under reduced pressure and dried azeotropically with toluene (3 mL×3) to give (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione, which was used directly in the next without further purification. LCMS (ESI): m/z: [M+H] calc'd for C₃₈H₄₅F₂N₅O₄ 673.3; found 674.5.

Step 10. To a mixture of (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (202 mg, 0.3 mmol) and (2S)-2-[(tert-butoxycarbonyl)(methyl)amino]-3-methylbutanoic acid (139 mg, 0.6 mmol) in THF under an atmosphere of Ar were added DIPEA (581 mg, 4.5 mmol), EDCI (86 mg, 0.45 mmol) and HOBt (61 mg, 0.45 mmol). The mixture was stirred for 16 h, then H₂O (100 mL) added and the mixture extracted with EtOAc (200 mL×3). The combined organic layers were washed with brine (50 mL×3), dried over anhydrous Na₂SO₄ and filtered. The filtrate was concentrated under reduced pressure and the residue was purified by silica gel column chromatography to give tert-butyl ((2S)-1-(((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (135 mg, 46% yield) as a solid. LCMS (ESI): m/z: [M+H] calc'd for C₄₉H₆₄F₂N₆O₇ 886.5; found 887.6.

Step 11. To a mixture of tert-butyl ((2S)-1-(((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (130 mg, 0.15 mmol) in DCM at 0° C. under an atmosphere of N₂ was added TFA (1.0 mL) dropwise. The mixture was stirred at 0° C. for 1.5 h, then concentrated under reduced pressure and dried azeotropically with toluene (3 mL×3) to give (2S)—N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (130 mg), which was used directly in the next step without further purification. LCMS (ESI): m/z: [M+H] calc'd for C₄₄H₅₆F₂N₆O₅ 786.4; found 787.6.

Step 12. To a mixture of (2S)—N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (130 mg, 0.17 mmol) and (3S)-1-(prop-2-enoyl)pyrrolidine-3-carboxylic acid (56 mg, 0.33 mmol) in MeCN (1.5 mL) at 0° C. under an atmosphere of N₂ were added DIPEA (427 mg, 3.3 mmol) and CIP (69 mg, 0.25 mmol). The mixture was stirred at 0° C. for 1 h, then H₂O (100 mL) was added and the mixture extracted with EtOAc (200 mL×3). The combined organic layers were washed with brine (50 mL×3), dried over anhydrous Na₂SO₄ and filtered. The filtrate was concentrated under reduced pressure and the residue was purified by preparative-HPLC to give (3S)-1-acryloyl-N-((2S)-1-(((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (58 mg, 36% yield) as a solid. LCMS (ESI): m/z: [M+H] calc'd for C₅₂H₆₅F₂N₇O₇ 937.4; found 938.1; ¹H NMR (400 MHz, DMSO-d₆) δ 8.78 (dd, J=4.8, 1.7 Hz, 1H), 8.43-8.21 (m, 1H), 8.02 (s, 2H), 7.93-7.81 (m, 2H), 7.76 (dd, J=9.3, 3.9 Hz, 1H), 7.66 (d, J=8.7 Hz, 1H), 7.56 (dd, J=7.7, 4.8 Hz, 1H), 7.34 (d, J=5.4 Hz, 1H), 7.20-6.86 (m, 1H), 6.80-6.40 (m, 1H), 6.15 (ddt, J=16.8, 4.9, 2.4 Hz, 1H), 5.90-5.60 (m, 1H), 5.59-5.19 (m, 2H), 4.71 (dd, J=10.7, 3.1 Hz, 1H), 4.40-4.17 (m, 3H), 4.12-3.90 (m, 3H), 3.85-3.71 (m, 1H), 3.61 (tdd, J=23.4, 9.9, 4.3 Hz, 6H), 3.40-3.30 (m, 2H), 3.11 (d, J=6.8 Hz, 3H), 3.08-2.90 (m, 2H), 2.87 (s, 2H), 2.84 (s, 3H), 2.69-2.30 (d, J=16.5 Hz, 1H), 2.30-1.79 (m, 5H), 1.75-1.45 (m, 2H), 1.40 (d, J=6.1 Hz, 3H), 1.05-0.85 (m, 6H), 0.85-0.66 (m, 6H), 0.57 (d, J=11.8 Hz, 3H).

### Example A741. Synthesis of (3S)-1-acryloyl-N-((2S)-1-(((2³S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-piperidinacycloundecaphane-4-yl)amino)-3-methyl-1-oxobutan-2-yl)-N-methylpyrrolidine-3-carboxamide

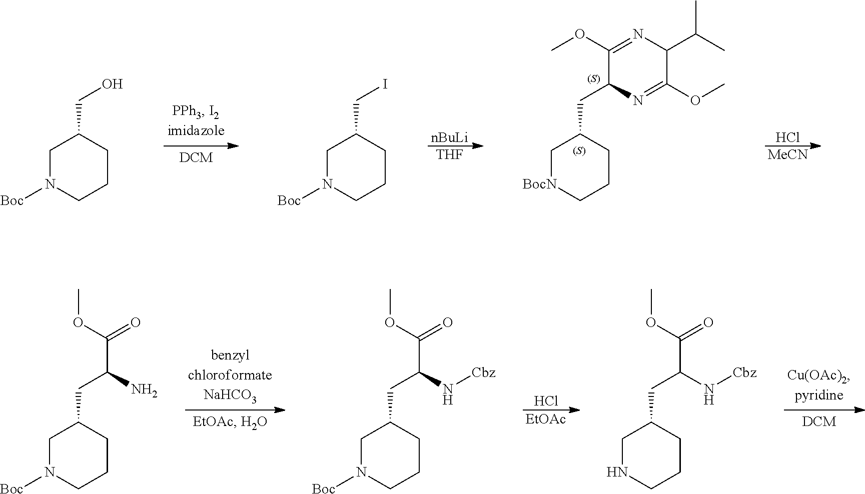

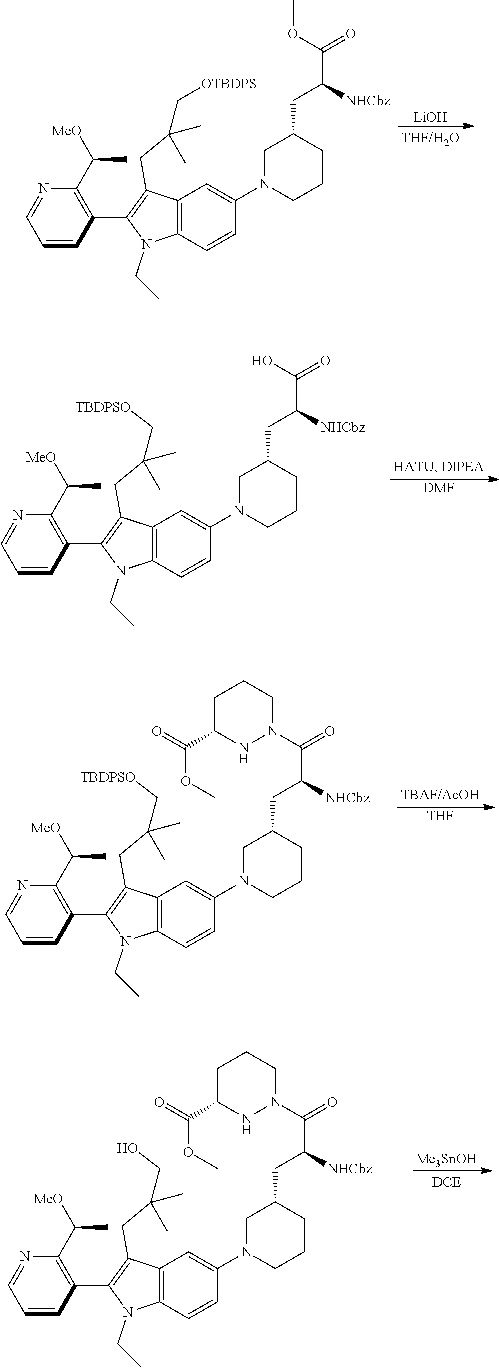

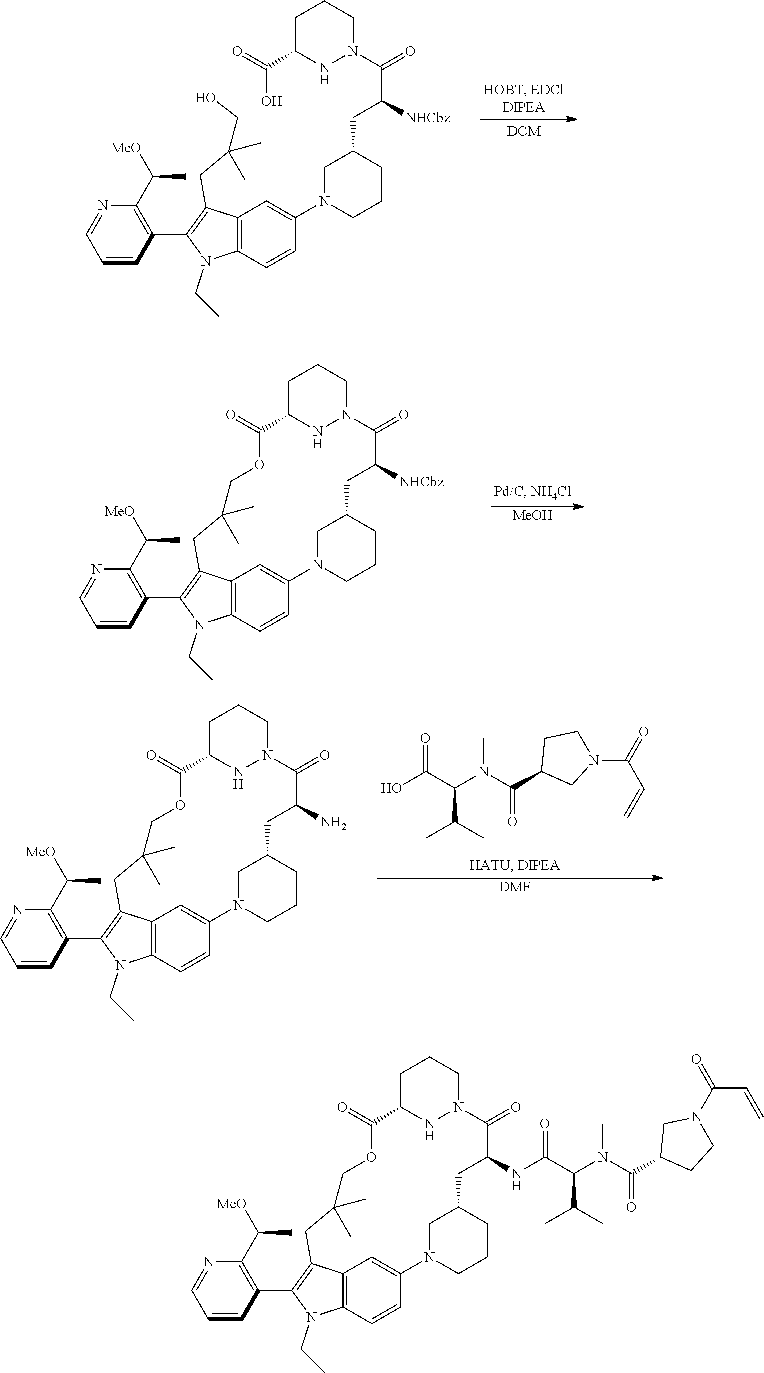

Step 1. A solution of tert-butyl (3R)-3-(hydroxymethyl) piperidine-1-carboxylate (10 g, 46.45 mmol) in DCM (200 mL) at 0° C., was added PPh₃ (15.8 g, 60.4 mmol), Imidazole (4.7 g, 69.7 mmol) and 12 (14.1 g, 55.74 mmol). The reaction suspension was stirred at 20° C. for 17 h, then concentrated under reduced pressure. The residue was purified by silica gel column chromatography to afford tert-butyl (3R)-3-(iodomethyl) piperidine-1-carboxylate (10 g, 66% yield) as oil. LCMS (ESI): m/z: [M+H] calc'd for C₁₁H₂₀INO₂ 325.1; found no mass.

Step 2. To a mixture of 3-isopropyl-2,5-dimethoxy-3,6-dihydropyrazine (10.8 g, 58.9 mmol) in THF (150 mL) at −60° C. under an atmosphere of N₂ was added n-BuLi (47 mL, 2.5 M in hexane, 117.7 mmol) dropwise. The mixture was warmed to 0° C. and was stirred for 2 h, then re-cooled to −60° C., and a solution of tert-butyl (3R)-3-(iodomethyl)piperidin-1-yl formate (9.60 g, 29.4 mmol) in THF (50 mL) was slowly added dropwise. The mixture was stirred at −60° C. for 2 h then warmed to room temperature and stirred for 2 h. Saturated NH₄Cl (150 mL) was slowly added and the mixture extracted with EtOAc (150 mL×2). The combined organic layers were washed with brine (200 mL), dried over anhydrous Na₂SO₄ and filtered. The filtrate was reduced under reduced pressure and the residue was purified by silica gel column chromatography to give tert-butyl (3S)-3-{[(2S)-5-isopropyl-3,6-dimethoxy-2,5-dihydropyrazin-2-yl]methyl}piperidin-1-yl formate (5.3 g, 46% yield) as a gum. LCMS (ESI): m/z: [M+H] calc'd for C₂₀H₃₅N₃O₄ 381.5; found 382.3.

Step 3. A mixture of tert-butyl (3S)-3-{[(2S)-5-isopropyl-3,6-dimethoxy-2,5-dihydropyrazin-2-yl]methyl}piperidin-1-yl formate (5.30 g, 13.9 mol) in MeCN (4 mL) was added 1M HCl (27.7 mL, 27.7 mmol) dropwise. The mixture was stirred for 2 h, then saturated NaHCO₃ until ˜pH 7-8, then extracted with DCM (30 mL×2). The combined organic layers was dried over anhydrous Na₂SO₄, filtered and concentrated under reduced pressure to give methyl (S)-tert-butyl 3-((S)-2-amino-3-methoxy-3-oxopropyl)piperidine-1-carboxylate (4.3 g, 95% yield) as an oil, which was used in next step without further purification. LCMS (ESI): m/z: [M+H] calc'd for C₁₄H₂₆N₂O₄ 286.2; found 287.3.

Step 4. To a mixture of methyl (S)-tert-butyl 3-((S)-2-amino-3-methoxy-3-oxopropyl)piperidine-1-carboxylate (4.30 g, 15.0 mmol) in EtOAc (30 mL) and H₂O (20 mL) at −10° C. was added NaHCO₃ (3.77 g, 44.88 mmol). The mixture was stirred at −10° C. for 10 min, then a solution of benzyl chloroformate (3.83 g, 22.44 mmol) was added dropwise. The mixture was warmed to 0° C. and stirred for 1 h, then H₂O (50 mL) was added and the mixture extracted with EtOAc (50 mL×2). The combined organic layers were dried over anhydrous Na₂SO₄, filtered, the filtrate was concentrated under reduced pressure to give tert-butyl (3S)-3-[(2S)-2-{[(benzyloxy)carbonyl] amino}-3-methoxy-3-oxopropyl]piperidine-1-carboxylate (4.0 g, 60% yield) as a gum. LCMS (ESI): m/z: [M−Boc+H] calc'd for C₁₇H₂₄N₂O₄ 320.2; found 321.3.

Step 5. To a mixture of tert-butyl (3S)-3-[(2S)-2-{[(benzyloxy)carbonyl] amino}-3-methoxy-3-oxopropyl]piperidine-1-carboxylate (1.0 g, 2.38 mmol) in EtOAc (8 mL) was added 2M HCl in EtOAc (11.9 mL, 23.8 mmol). The mixture was stirred for 2 h, then saturated NaHCO₃ added until ˜pH 8-9, and the mixture extracted with DCM (30 mL×3). The combined organic layer was dried over anhydrous Na₂SO₄, filtered and concentrated under reduced pressure to give (2S)-2-{[(benzyloxy)carbonyl]amino}-3-[(3S)-piperidin-3-yl]propanoate (740 mg, 91% yield) as a gum. LCMS (ESI): m/z: [M+H] calc'd for C₁₇H₂₄N₂O₄ 320.2; found 321.2.

Step 6. To a mixture of (3-{3-[(tert-butyldiphenylsilyl)oxy]-2,2-dimethylpropyl}-1-ethyl-2-{2-[(1S)-1-methoxyethyl]pyridin-3-yl}indol-5-yl)boranediol (5.47 g, 8.43 mmol) and methyl (2S)-2-{[(benzyloxy)carbonyl]amino}-3-[(3S)-piperidin-3-yl]propanoate (2.70 g, 8.43 mmol) in DCM (70 mL) was added Cu(OAc)₂ (6.06 g, 16.86 mmol) and pyridine (2.0 g, 25.3 mmol). The mixture was stirred under an atmosphere of 02 for 48 h, then diluted with DCM (200 mL) and washed with H₂O (150 mL×2). The organic layer was dried over anhydrous Na₂SO₄, filtered, concentrated under reduced pressure and the residue was purified by silica gel column chromatography to give methyl 2-{[(benzyloxy)carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl)piperidin-3-yl]propanoate (3.6 g, 42% yield) as a solid. LCMS (ESI): m/z: [M/2+H] calc'd for C₅₆H₇₀N₄O₆Si 462.3; found 462.3.

Step 7. To a mixture of methyl 2-{[(benzyloxy)carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl)piperidin-3-yl]propanoate (3.60 g, 3.57 mmol) in THF (60 mL) and H₂O (30 mL) was added LiOH (342 mg, 14.28 mmol). The mixture was stirred for 2 h, then diluted with H₂O (150 mL), then 1M HCl was added slowly until ˜pH 3-4 and the mixture extracted with EtOAc (200 mL×3). The combined organic layers were dried over anhydrous Na₂SO₄, filtered and the filtrate concentrated under reduced pressure to give 2-{[(benzyloxy)carbonyl]amino}-3-[(3S)-1-(3-{3-[(tert-butyidiphenylsilyl)oxy]-2,2-dimethylpropyl}-1-ethyl-2-{2-[(1S)-1-methoxyethyl]pyridin-3-yl}indol-5-yl)piperidin-3-yl]propanoic acid (3.3 g, 85% yield) as a solid, which was used directly in the next step without further purification. LCMS (ESI): m/z: [M/2+H] calc'd for C₅₅H₆₈N₄O₆Si 455.3; found 455.3.

Step 8. To a mixture of methyl 2-{[(benzyloxy)carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl)piperidin-3-yl]propanoate (3.30 g, 2.91 mmol) in DMF (40 mL) was added methyl (3S)-1,2-diazinane-3-carboxylate (0.42 g, 2.91 mmol), HATU (2.21 g, 5.82 mmol) and DIPEA (2.26 g, 17.46 mmol). The mixture was stirred for 3 h, then poured into ice-H₂O and extracted with EtOAc (120 mL×2). The combined organic layers were washed with saturated NaHCO₃ (150 mL), brine (150 mL), dried over anhydrous Na₂SO₄ and filtered. The filtrate was concentrated under reduced pressure and the residue was purified by silica gel column chromatography to give methyl (3S)-1-[(2S)-2-{[(benzyloxy)carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl)piperidin-3-yl]propanoyl]-1,2-diazinane-3-carboxylate (2.9 g, 95% yield) as a gum. LCMS (ESI): m/z: [M/2+H] calc'd for C₆₁H₇₈N₆O₇Si 518.3; found 518.3.

Step 9. To a mixture of methyl (3S)-1-[(2S)-2-{[(benzyloxy)carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl)piperidin-3-yl]propanoyl]-1,2-diazinane-3-carboxylate (1.70 g, 1.64 mmol) was added a mixture of 1M TBAF in THF (19.68 mL, 19.68 mmol) and AcOH (1.18 g, 19.68 mmol). The reaction was heated to 60° C. and stirred for 22 h, then diluted with EtOAc (80 mL) and washed with saturated NaHCO₃ (80 mL), H₂O (60 mL×2) and brine (60 mL). The organic layer was dried over anhydrous Na₂SO₄, filtered, the filtrate was concentrated under reduced pressure and the residue was purified by preparative-HPLC to give methyl (3S)-1-[(2S)-2-{[(benzyloxy)carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylate (1.0 g, 73% yield) as a solid. LCMS (ESI): m/z: [M/2+H] calc'd for C₄₅H₆₀N₆O₇ 399.2; found 399.4.

Step 10. To a mixture m methyl (3S)-1-[(2S)-2-{[(benzyloxy)carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylate (1.0 g, 1.1 mmol) in 1,2-dichloroethane (10 mL) was added Me₃SnOH (1.42 g 7.84 mmol). The mixture was heated to 65° C. and stirred for 10 h, then filtered and the filtrate was concentrated under reduced pressure to give (3S)-1-[(2S)-2-{[(benzyloxy)carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylic acid (1.0 g, 99% yield) as a gum. The product was used in the next step without further purification. LCMS (ESI): m/z: [M/2+H] calc'd for C₄₄H₅₆N₆O₇ 392.2; found 392.3.

Step 11. To a mixture of (3S)-1-[(2S)-2-{[(benzyloxy)carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylic acid (1.0 g, 1.1 mmol) in DCM (30 mL) at 0° C. was added HOBT (1.51 g, 11.2 mmol), N-(3-dimethylaminopropyl)-N-ethylcarbodiimide HCl (6.44 g, 33.6 mmol) and DIPEA (5.79 g, 44.8 mmol). The mixture was warmed to room temperature and stirred for 6 h, then diluted with H₂O and extracted with EtOAc (100 mL×3). The combined organic layers were dried over anhydrous Na₂SO₄, filtered, the filtrate was concentrated under reduced pressure and the residue was purified by silica gel column chromatography to give benzyl N-[(6S,8S,14S)-22-ethyl-21-{2-[(1S)-1-methoxyethyl]pyridin-3-yl}-18,18-dimethyl-9,15-dioxo-16-oxa-2,10,22,28-tetraazapentacyclo [18.5.2.1\hat{ }{2,6}.1\hat{ }{10, 14}.0\hat{ }{23,27}]nonacosa-1(26),20,23(27),24-tetraen-8-yl]carbamate (340 mg, 36% yield) as a solid. LCMS (ESI): m/z: [M+H] calc'd for C₄₄H₅₆N₆O₆ 383.2; found 383.3.

Step 12. A mixture of benzyl N-[(6S,8S,14S)-22-ethyl-21-{2-[(1S)-1-methoxyethyl]pyridin-3-yl}-18,18-dimethyl-9,15-dioxo-16-oxa-2,10,22,28-tetraazapentacyclo [18.5.2.1\hat{ }{2,6}.1\hat{ }{10, 14}.0\hat{ }{23,27}]nonacosa-1(26),20,23(27),24-tetraen-8-yl]carbamate (250 mg, 0.33 mmol), Pd/C (100 mg) and NH₄Cl (353 mg, 6.6 mmol) in MeOH (5 mL) was stirred under an atmosphere of H₂ for 4 h. The mixture was filtered through Celite and the filtrate was concentrated under reduced pressure. The residue was dissolved in DCM (30 mL) and washed with saturated NaHCO₃ (20 mL), H₂O (20 mL) and brine (20 mL). The organic layer was dried over Na₂SO₄, filtered, and the filtrate concentrated under reduced pressure to give (6S,8S,14S)-8-amino-22-ethyl-21-{2-[(1S)-1-methoxyethyl]pyridin-3-yl}-18,18-dimethyl-16-oxa-2,10,22,28-tetraazapentacyclo[18.5.2.1\hat{ }{2,6}.1\hat{ }{10,14}.0\hat{ }{23,27}]nonacosa-1(26),20,23(27),24-tetraene-9,15-dione, which was used in next step without further purification. LCMS (ESI): m/z: [M+H] calc'd for C₃₆H₅₀N₆O₄ 631.4; found 631.4.

Step 13. To a mixture of (6S,8S,14S)-8-amino-22-ethyl-21-{2-[(1S)-1-methoxyethyl]pyridin-3-yl}-18,18-dimethyl-16-oxa-2,10,22,28-tetraazapentacyclo[18.5.2.1\hat{ }{2,6}.1\hat{ }{10,14}.0\hat{ }{23,27}]nonacosa-1(26),20,23(27),24-tetraene-9,15-dione (300 mg, 0.48 mmol), (2S)-3-methyl-2-{N-methyl-1-[(3S)-1-(prop-2-enoy)pyrrolidin-3-yl]formamido}butanoic acid (136 mg, 0.48 mmol) and DIPEA (620 mg, 4.8 mmol) in DMF (5 mL) at 0° C. was added HATU (183 mg, 0.48 mmol). The mixture was stirred at 0-5° C. for 1 h, then diluted with EtOAc (50 mL), washed with H₂O (50 mL×2), brine (50 mL), dried over Na₂SO₄ and filtered. The filtrate was concentrated under reduced pressure and the residue was purified by silica gel column chromatography to give (2S)—N-[(6S,8S,14S)-22-ethyl-21-{2-[(1S)-1-methoxyethy]pyridin-3-yl}-18,18-dimethyl-9,15-dioxo-16-oxa-2,10,22,28-tetraazapentacyclo[18.5.2.1\hat{ }{2,6}.1\hat{ }{10,14}.0\hat{ }{23,27}]nonacosa-1(26),20,23(27),24-tetraen-8-yl]-3-methyl-2-{N-methyl-1-[(3S)-1-(prop-2-enoyl)pyrrolidin-3-yl]formamido}butanamide (90 mg, 20% yield) as a solid. LCMS (ESI): m/z: [M+H] calc'd for C₅₀H₇₀N₈O₇ 895.5; found 895.4; ¹H NMR (400 MHz, CD₃OD) δ 8.71 (dd, J=4.8, 1.5 Hz, 1H), 7.86 (dd, J=7.7, 1.5 Hz, 1H), 7.51 (dd, J=7.7, 4.8 Hz, 1H), 7.36 (dd, J=8.9, 1.9 Hz, 1H), 7.22 (d, J=12.1 Hz, 1H), 7.09 (dd, J=8.9, 1.9 Hz, 1H), 6.59 (dt, J=16.9, 9.9 Hz, 1H), 6.26 (ddd, J=16.8, 5.0, 1.9 Hz, 1H), 5.80-5.67 (m, 1H), 5.59-5.46 (m, 1H), 4.93 (d, J=12.4 Hz, 1H), 4.66 (dd, J=11.1, 6.4 Hz, 1H), 4.45 (d, J=12.6 Hz, 1H), 4.28-4.19 (m, 1H), 4.13 (dd, J=14.5, 7.2 Hz, 1H), 4.02-3.87 (m, 1H), 3.87-3.36 (m, 1H), 3.16 (s, 2H), 3.10 (d, J=3.4 Hz, 2H), 2.76 (dd, J=26.9, 13.5 Hz, 3H), 2.61 (s, 1H), 2.35-1.97 (m, 5H), 1.78 (dd, J=25.4, 22.1 Hz, 10H), 1.45 (d, J=6.2 Hz, 3H), 1.04 (d, J=6.2 Hz, 3H), 0.95 (dd, J=6.5, 1.8 Hz, 3H), 0.83 (d, J=6.6 Hz, 3H), 0.72 (d, J=31.8 Hz, 6H).

### Example A715. Synthesis of benzyl ((2³S,6S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-piperidinacycloundecaphane-4-yl)carbamate

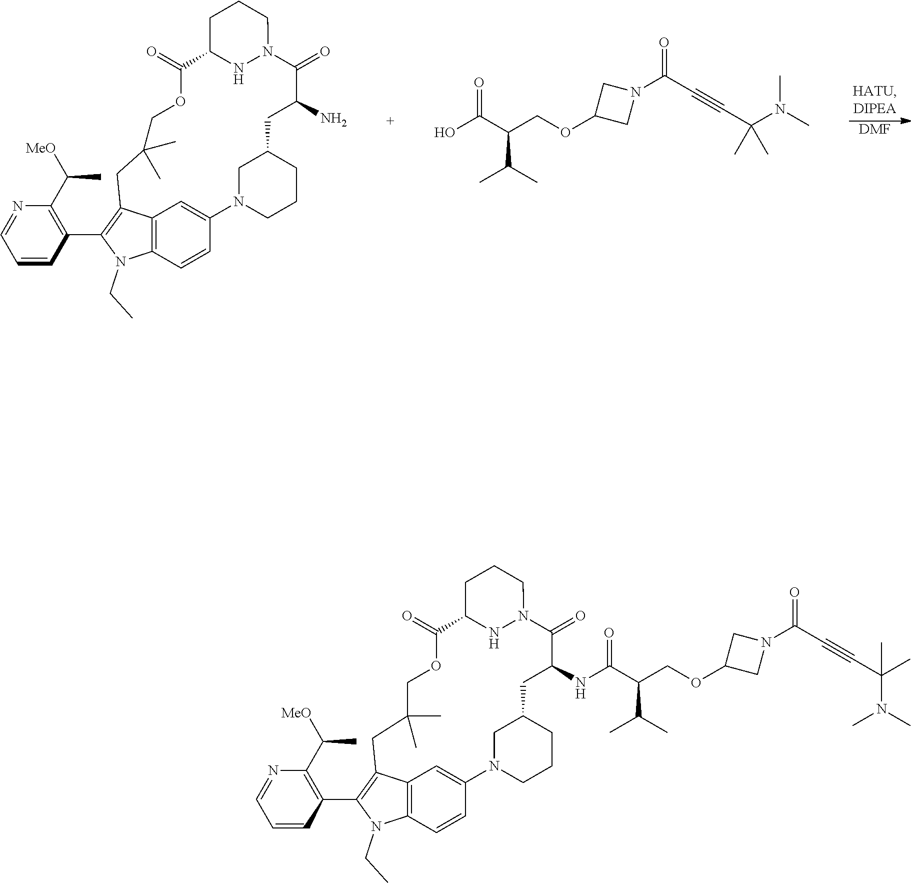

To a solution of ((2³S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-piperidinacycloundecaphane-5,7-dione (50 mg, 0.08 mmol), (R)-2-(((1-(4-(dimethylamino)-4-methylpent-2-ynoyl)azetidin-3-yl)oxy)methyl)-3-methylbutanoic acid (26 mg, 0.08 mmol) and DIPEA (31 mg, 0.24 mmol) in DMF (1 mL) at 0° C., was added HATU (30 mg, 0.08 mmol). The reaction mixture was stirred at 0-5° C. for 1 h, then diluted with EtOAc (20 mL), washed with H₂O (20 mL×2) and brine (20 mL). The organic phase was separated and dried over anhydrous sodium sulfate, filtered and concentrated under reduced pressure. The residue was purified by silica gel column chromatography to give (2R)-2-(((1-(4-(dimethylamino)-4-methylpent-2-ynoyl)azetidin-3-yl)oxy)methyl)-N-((2³S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-piperidinacycloundecaphane-4-yl)-3-methylbutanamide as solid. ¹H NMR (400 MHz, CD₃OD) δ 8.71 (d, J=4.7 Hz, 1H), 8.24 (s, 1H), 8.10 (dd, J=26.7, 7.8 Hz, 1H), 7.86 (d, J=7.7 Hz, 1H), 7.51 (dd, J=7.7, 4.8 Hz, 1H), 7.39 (dd, J=8.9, 3.1 Hz, 1H), 7.26 (d, J=16.6 Hz, 1H), 7.11 (d, J=8.8 Hz, 1H), 5.61 (s, 1H), 4.50-4.28 (m, 3H), 4.27-4.07 (m, 3H), 3.98 (ddd, J=25.6, 13.4, 5.1 Hz, 2H), 3.84-3.72 (m, 2H), 3.62 (dd, J=10.7, 4.8 Hz, 2H), 3.55 (d, J=7.1 Hz, 2H), 3.47 (d, J=6.5 Hz, 2H), 3.16 (s, 3H), 3.03-2.91 (m, 1H), 2.76 (dd, J=28.7, 15.2 Hz, 3H), 2.62 (s, 1H), 2.40 (t, J=7.0 Hz, 3H), 2.33 (dd, J=14.3, 5.0 Hz, 4H), 2.05 (d, J=11.6 Hz, 1H), 1.99-1.64 (m, 1 OH), 1.64-1.55 (m, 1H), 1.51-1.42 (m, 6H), 1.37 (d, J=12.3 Hz, 3H), 1.05 (s, 3H), 0.94 (ddd, J=9.3, 6.7, 2.0 Hz, 6H), 0.76 (d, J=3.8 Hz, 3H), 0.69 (s, 3H). LCMS (ESI): m/z: [M+H] calc'd for C₅₃H₇₈N₈O₇ 936.6; found 937.4.

### Example A347. Synthesis of (2S)—N-[(7S,13S)-21-ethyl-20-{2-[(1S)-1-methoxyethyl]pyridin-3-yl}-17,17-dimethyl-8,14-dioxo-15-oxa-4-thia-9,21,25,27,28-pentaazapentacyclo[17.5.2.1\hat{ }{2,5}.1\hat{ }{9,13}.0\hat{ }{22,26}]octacosa-1(25),2,5(25),19,22(26),23-hexaen-7-yl]-3-methy-2-{N-methyl-1-[(3S)-1-(prop-2-enoyl)pyrrolidin-3-yl]formamido}butanamide

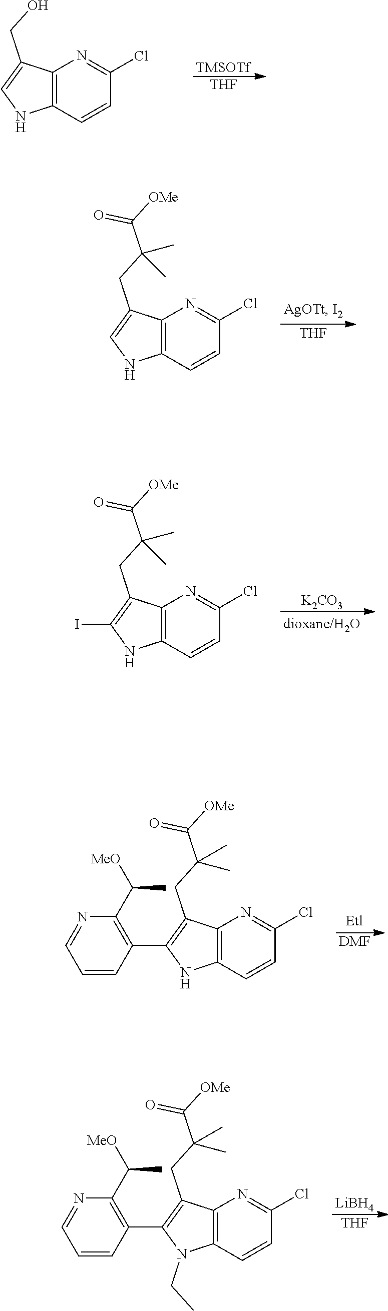

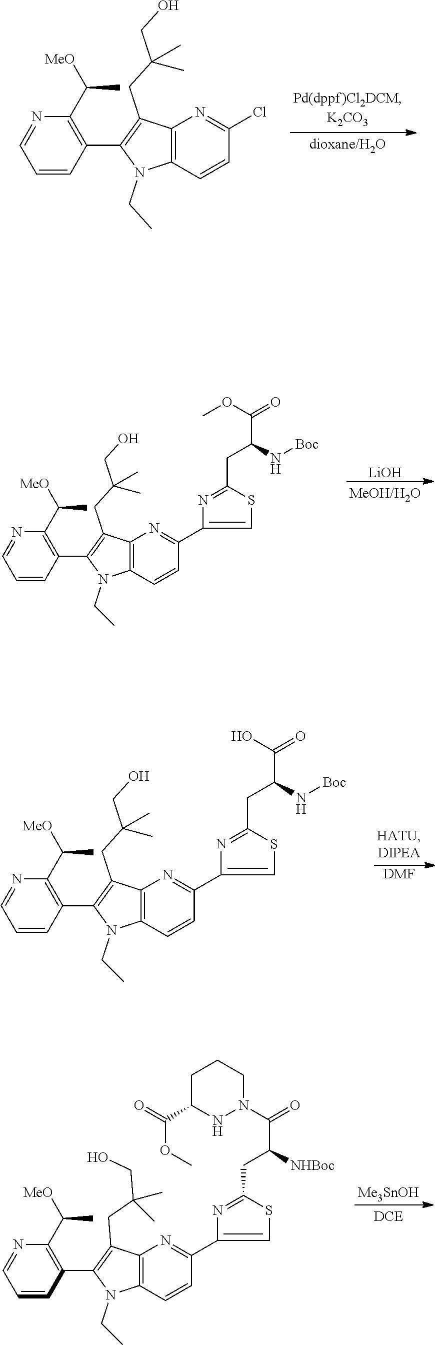

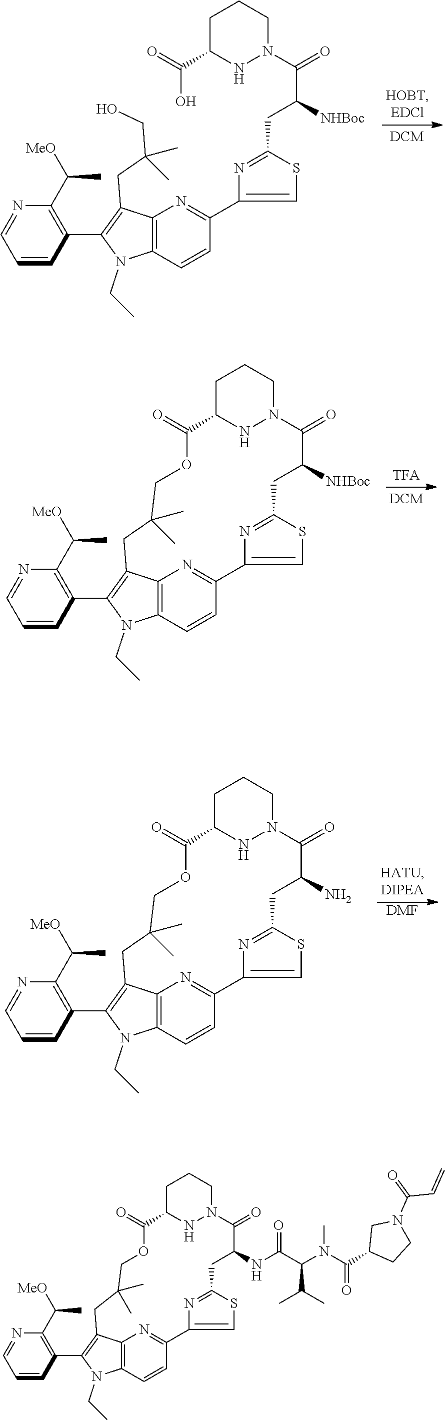

Step 1. To a mixture of (5-chloro-1H-pyrrolo[3,2-b]pyridin-3-yl)methanol (3.5 g, 19 mmol), and ((1-methoxy-2-methylprop-1-en-1-yl)oxy)trimethylsilane (6.7 g, 38 mmol) in THF (50 ml) at 0° C. was added TMSOTf (3.8 g, 17 mmol) dropwise. The mixture was stirred at 0-5° C. for 2 h, then diluted with EtOAc (100 mL) and washed with saturated NaHCO₃ (50 mL) and brine (50 mL×2). The organic layer was dried over Na₂SO₄, filtered and the filtrate was concentrated under reduced pressure. The residue was purified by silica gel column chromatography to give methyl 3-(5-chloro-1H-pyrrolo[3,2-b]pyridin-3-yl)-2,2-dimethylpropanoate (3.0 g, 59% yield) as a solid. LCMS (ESI): m/z: [M+H] calc'd for C₁₃H₁₅ClN₂O₂ 266.1; found 267.1.

Step 2. To a mixture of methyl 3-(5-chloro-1H-pyrrolo[3,2-b]pyridin-3-yl)-2,2-dimethylpropanoate (3.0 g, 11 mmol) in anhydrous THF (50 mL) at 0° C. was added AgOTf (4.3 g, 17 mmol) and 12 (2.9 g, 11 mmol). The mixture was stirred at 0° C. for 2 h, then saturated Na₂SO₃ (20 mL) and EtOAc (50 mL) added. The mixture was filtered and the filtrate was washed with brine (50 mL), dried over Na₂SO₄ and filtered. The filtrate was concentrated under reduced pressure and the residue was purified by silica gel column chromatography to give methyl 3-(5-chloro-2-iodo-1H-pyrrolo[3,2-b]pyridin-3-yl)-2,2-dimethylpropanoate (2.3 g, 52% yield) as a solid. LCMS (ESI): m/z: [M+H] calc'd for C₁₃H₁₅ClIN₂O₂ 392.0; found 393.0.

Step 3. To a mixture of methyl 3-(5-chloro-2-iodo-1H-pyrrolo[3,2-b]pyridin-3-yl)-2,2-dimethylpropanoate (2.3 g, 5.9 mmol) and 2-(2-(2-methoxyethyl)phenyl)-4,4,5,5-tetramethyl-1,3,2-dioxaborolane (1.6 g, 7.1 mmol) and K₂CO₃ (2.4 g, 18 mol) in 1,4-dioxane (25 mL) and H₂O (5 mL) under an atmosphere of N₂ was added Pd(dppf)Cl₂·DCM (480 mg, 0.59 mmol). The mixture was heated to 70° C. and for 4 h, then diluted with EtOAc (200 mL) and washed with brine (25 mL), dried over Na₂SO₄ and filtered. The filtrate was concentrated under reduced pressure and the residue was purified by silica gel column chromatography to give methyl (S)-3-(5-chloro-2-(2-(1-methoxyethyl)pyridin-3-yl)-1H-pyrrolo[3,2-b]pyridin-3-yl)-2,2-dimethylpropanoate (2.0 g, 84% yield) as a solid. LCMS (ESI): m/z: [M+H] calc'd for C₂₁H₂₄ClN₃O₃ 401.2; found 402.2.

Step 4. A mixture of ethyl 3-(5-chloro-2-(2-((S)-1-methoxyethyl)pyridin-3-yl)-1H-pyrrolo[3,2-b]pyridin-3-yl)-2-methylpropanoate (2.0 g, 5.0 mmol), Cs₂CO₃ (3.3 g, 10 mmol) and EtI (1.6 g, 10 mmol) in DMF (30 mL) was stirred for 10 h. The mixture was diluted with EtOAc (100 mL) and washed with brine (20 mL×4), dried over Na₂SO₄ and filtered. The filtrate was concentrated under reduced pressure and the residue was purified by silica gel column chromatography to give two diastereomers of methyl (S)-3-(5-chloro-1-ethyl-2-(2-(1-methoxyethyl)pyridin-3-yl)-1H-pyrrolo[3,2-b]pyridin-3-yl)-2,2-dimethylpropanoate (0.7 g, 32% yield; 0.6 g, 28% yield) as a solid. LCMS (ESI): m/z: [M+H] calc'd for C₂₃H₂₈ClN₃O₃ 429.2; found 430.2.

Step 5. To a mixture of methyl (S)-3-(5-chloro-1-ethyl-2-(2-(1-methoxyethyl)pyridin-3-yl)-1H-pyrrolo[3,2-b]pyridin-3-yl)-2,2-dimethylpropanoate (1.9 g, 4.4 mmol) in anhydrous THF (20 mL) at 0° C. was added LiBH₄ (200 mg, 8.8 mmol). The mixture was heated to 60° C. and stirred for 4 h, then saturated NH₄Cl (20 mL) and EtOAc (50 mL) added. The aqueous and organic layers were separated and the organic layer was washed with brine (30 mL), dried over Na₂SO₄ and filtered. The filtrate was concentrated under reduced pressure and the residue was purified by silica gel column chromatography to give (S)-3-(5-chloro-1-ethyl-2-(2-(1-methoxyethyl)pyridin-3-yl)-1H-pyrrolo[3,2-b]pyridin-3-yl)-2,2-dimethylpropan-1-ol (1.5 g, 85% yield) as a solid. LCMS (ESI): m/z: [M+H] calc'd for C₂₂H₂₈ClN₃O₂ 401.2; found 402.2.

Step 6. To a mixture of (S)-3-(5-chloro-1-ethyl-2-(2-(1-methoxyethyl)pyridin-3-yl)-1H-pyrrolo[3,2-b]pyridin-3-yl)-2,2-dimethylpropan-1-ol (550 mg, 1.37 mmol), (S)-(2-(2-((tert-butoxycarbonyl)amino)-3-methoxy-3-oxopropyl)thiazol-4-yl)boronic acid (907.4 mg, 2.74 mmol, 2 eq) and K₂CO₃ (568 mg, 4.11 mmol) in 1,4-dioxane (25 mL) and H₂O (5 mL) under an atmosphere of N₂ was added Pd(dppf)Cl₂·DCM (89 mg, 0.14 mmol). The mixture was heated to 70° C. and stirred for 4 h, then H₂O (50 mL) added and the mixture extracted with EtOAc (100 mL×3). The combined organic layers were washed with brine (50 mL), dried over Na₂SO₄ and filtered. The filtrate was concentrated under reduced pressure and the residue was purified by silica gel column chromatography to give methyl (S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoate (440 mg, 22% yield) as a solid, which was used directly in the next step. LCMS (ESI): m/z: [M+H] calc'd for C₃₄H₄₅N₅O₆S 651.3; found 652.3.

Step 7. To a mixture of (2S)-methyl 2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoate (280 mg, 0.43 mmol) in MeOH (4 mL) was added a solution of LiOH (51 mg, 2.2 mmol) in H₂O (2 mL). The mixture was stirred for 5 h, then pH adjusted to ˜3-4 by addition of 1M HCl. The mixture was diluted with H₂O (30 mL) and extracted with EtOAc (15 mL×3). The combined organic layers were washed with brine (10 mL), dried over anhydrous Na₂SO₄, filtered and the filtrate was concentrated under reduced pressure to give (2S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoic acid (280 mg) as solid, which was used directly in the next step without further purification. LCMS (ESI): m/z: [M+H] calc'd for C₃₃H₄₃N₅O₆S 637.3; found 638.3.

Step 8. To a mixture of (2S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoic acid (274 mg, 0.43 mmol) and methyl (3S)-1,2-diazinane-3-carboxylate (280 mg, 0.64 mmol) in DMF (3 mL) at 0-5° C. was added a solution of HATU (245 mg, 0.64 mmol) and DIPEA (555 mg, 4.3 mmol) in DMF (2 mL). The mixture was stirred for 1 h, then diluted with EtOAc (20 mL) and H₂O (20 mL). The aqueous and organic layers were partitioned and the organic layer was washed with H₂O (20 mL×3), brine (20 mL), dried over anhydrous Na₂SO₄ and filtered. The filtrate was concentrated under reduced pressure and the residue was purified by silica gel column chromatography to give methyl (3S)-1-[(2S)-2-{[(tert-butoxy)carbonyl]amino}-3-{4-[1-ethyl-3-(3-hydroxy-2,2-dimethylpropyl)-2-{2-[(1S)-1-methoxyethyl]pyridin-3-yl}pyrrolo[3,2-b]pyridin-5-yl]-1,3-thiazol-2-yl}propanoyl]-1,2-diazinane-3-carboxylate (230 mg, 70% yield) as a solid. LCMS (ESI): m/z: [M+H] calc'd for C₃₉H₅₃N₇O₇S 763.4; found 764.3.

Step 9. To a mixture of methyl (3S)-1-[(2S)-2-{[(tert-butoxy)carbonyl]amino}-3-{4-[1-ethyl-3-(3-hydroxy-2,2-dimethylpropyl)-2-{2-[(1S)-1-methoxyethyl]pyridin-3-yl}pyrrolo[3,2-b]pyridin-5-yl]-1,3-thiazol-2-yl}propanoyl]-1,2-diazinane-3-carboxylate (230 mg, 0.3 mmol) in DCE (3 mL) under an atmosphere of N₂ was added Me₃SnOH (300 mg). The mixture was heated to 65° C. and stirred for 16 h, then concentrated under reduced pressure. The residue was diluted with EtOAc (20 mL), washed with H₂O (20 mL) and brine (10 mL), dried over Na₂SO₄ and filtered. The filtrate was concentrated under reduced pressure to give (3S)-1-[(2S)-2-{[(tert-butoxy)carbonyl]amino}-3-{4-[1-ethyl-3-(3-hydroxy-2,2-dimethylpropyl)-2-{2-[(1S)-1-methoxyethyl]pyridin-3-yl}pyrrolo[3,2-b]pyridin-5-yl]-1,3-thiazol-2-yl}propanoyl]-1,2-diazinane-3-carboxylic acid (200 mg) as a foam, which was used directly in the next step without further purification. LCMS (ESI): m/z: [M+H] calc'd for C₃₈H₅₁N₇O₇S 749.4; found 750.3.

Step 10. To a mixture of (3S)-1-[(2S)-2-{[(tert-butoxy)carbonyl]amino}-3-{4-[1-ethyl-3-(3-hydroxy-2,2-dimethylpropyl)-2-{2-[(1S)-1-methoxyethyl]pyridin-3-yl}pyrrolo[3,2-b]pyridin-5-yl]-1,3-thiazol-2-yl}propanoyl]-1,2-diazinane-3-carboxylic acid (245 mg, 0.32 mmol) in DCM (50 mL) at 0-5° C. were added HOBT (432 mg, 3.2 mmol), EDCI HCl (1.8 g, 9.6 mmol) and DIPEA (1.65 g, 12.8 mmol). The mixture was warmed to room temperature and stirred for 16 h, then concentrated under reduced pressure. The residue was diluted with EtOAc (20 mL) and H₂O (20 mL) and the aqueous and organic layers were partitioned. The organic layer was washed with H₂O (30 mL×3), brine (30 mL), dried over anhydrous Na₂SO₄ and filtered. The filtrate was concentrated under reduced pressure and the residue was purified by preparative-TLC to give tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-4-yl)carbamate (100 mg, 43% yield) as a solid. LCMS (ESI): m/z [M+H] calc'd for C₃₈H₄₉N₇O₆S 731.4; found 732.3.

Step 11. A mixture of tert-butyl ((6³S,4S,2-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-4-yl)carbamate (80 mg, 0.11 mmol) in DCM (0.6 mL) and TFA (0.2 mL) was stirred for 1 h. The mixture was concentrated under reduced pressure to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-5,7-dione (72 mg, 95% yield) as a solid. LCMS (ESI): m/z: [M+H] calc'd for C₃₃H₄₁N₇O₄S 631.3; found 632.3.

Step 12. To a mixture of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-pyrrolo[3,2-b]pyridina-6(1,3)- pyridazinacycloundecaphane-5,7-dione (120 mg, 0.39 mmol) and DIPEA (335 mg, 2.6 mmol) in DMF (1 mL) at 0° C. was added HATU (60 mg, 0.16 mmol). The mixture was stirred at 0° C. for 1 h, then diluted with H₂O (110 mL) and extracted with EtOAc (80 mL×2). The combined organic layers were washed with H₂O (100 mL), brine (100 mL), dried over anhydrous Na₂SO₄ and filtered. The filtrate was concentrated under reduced pressure and the residue was purified by preparative-TLC to give (3S)-1-acryloyl-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-4-yl)amino)-3-methyl-1-oxobutan-2-yl)-N-methylpyrrolidine-3-carboxamide (1.8 mg, 2% yield) as a solid. LCMS (ESI): m/z: [M+H] calc'd for C₄₇H₆₁N₉O₇S 895.4; found 896.3; ¹H NMR (400 MHz, CD₃OD) δ 8.72 (d, J=4.5 Hz, 1H), 7.98-7.77 (m, 3H), 7.72 (dd, J=12.0, 8.6 Hz, 1H), 7.54 (dd, J=7.6, 4.8 Hz, 1H), 6.67-6.54 (m, 1H), 6.26 (m, 1H), 5.79-5.58 (m, 2H), 4.83-4.75 (m, 1H), 4.39-4.16 (m, 4H), 4.02 (dd, J=28.0, 10.6 Hz, 2H), 3.89-3.65 (m, 6H), 3.50 (m, 4H), 3.34 (d, J=6.2 Hz, 3H), 3.12 (d, J=4.0 Hz, 2H), 3.00 (s, 1H), 2.73 (m, 1H), 2.48-2.37 (m, 1H), 2.31-2.07 (m, 4H), 1.88 (d, J=11.2 Hz, 1H), 1.71 (d, J=12.8 Hz, 1H), 1.44 (m, 7H), 0.97 (dd, J=6.2, 4.4 Hz, 3H), 0.92-0.84 (m, 8H), 0.41 (d, J=6.2 Hz, 3H).

### Example 647. Synthesis of 1-(4-(dimethylamino)-4-methylpent-2-ynoyl)-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-4-fluoro-N-methylpiperidine-4-carboxamide

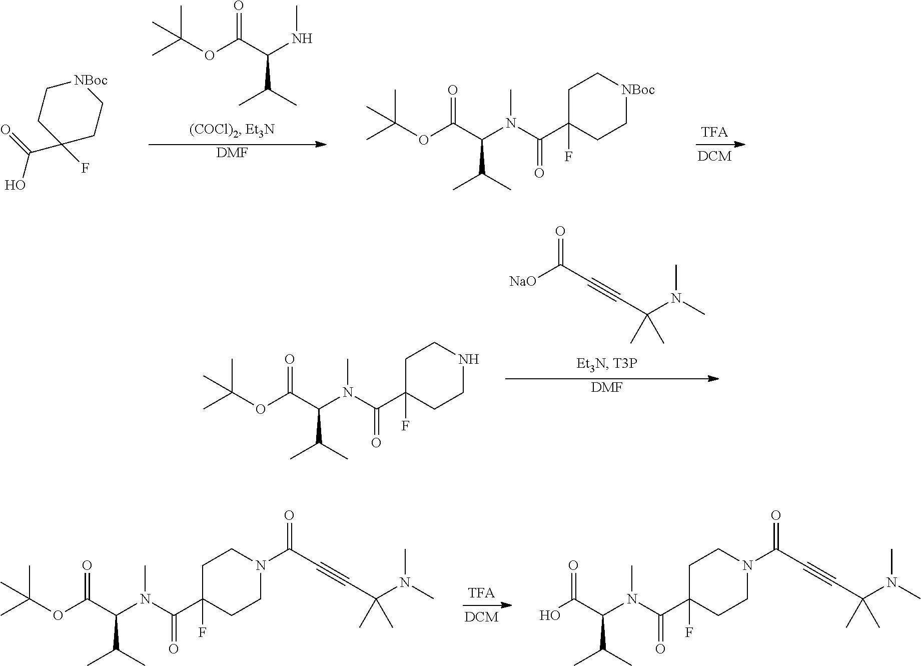

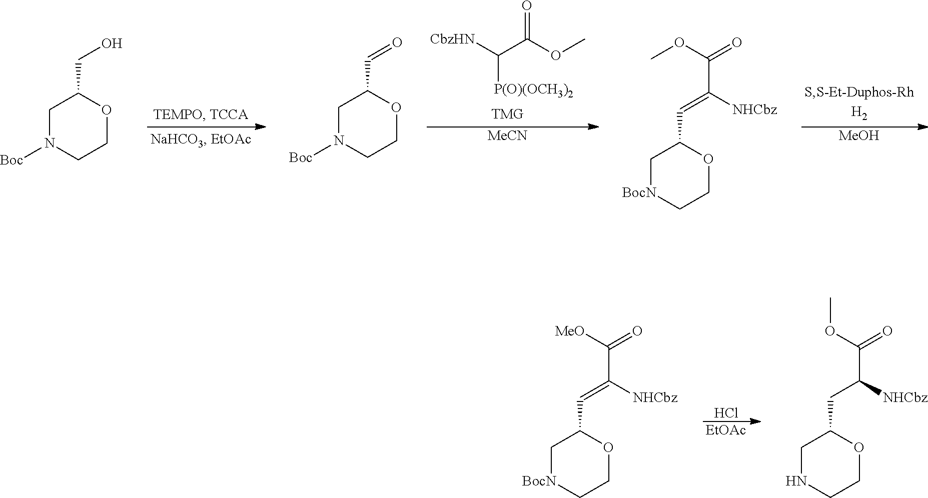

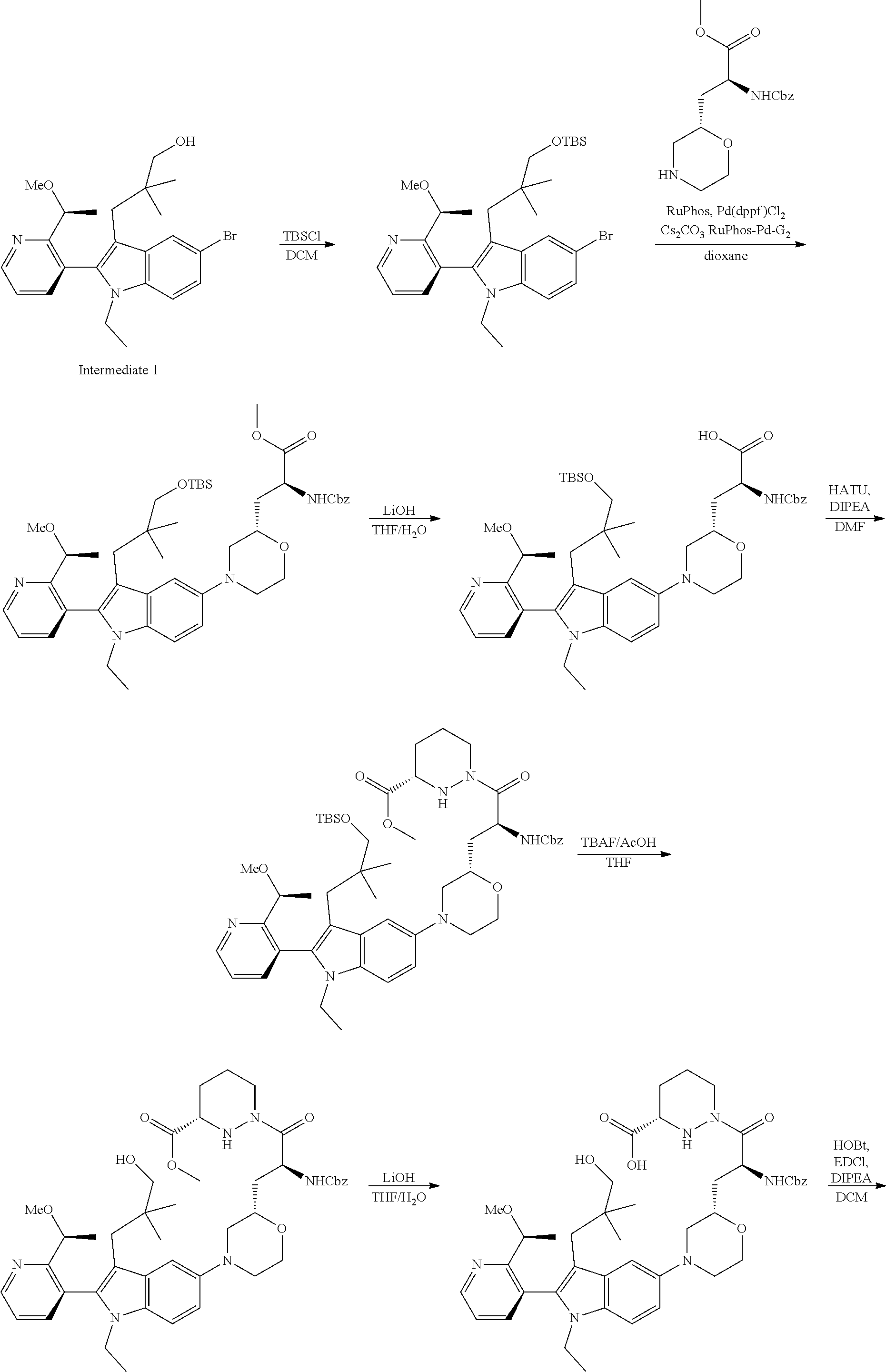

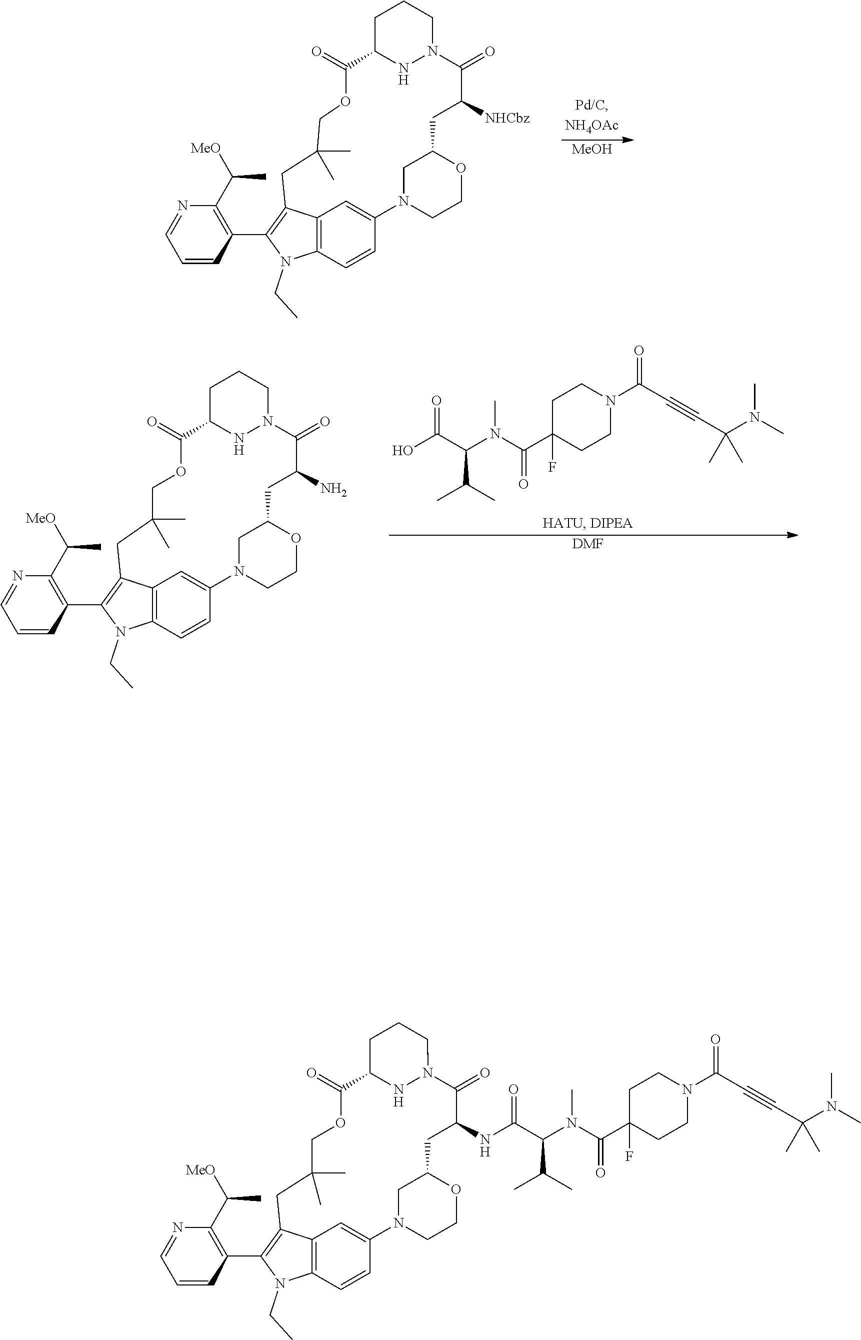

Step 1. A mixture of 1-[(tert-butoxy)carbonyl]-4-fluoropiperidine-4-carboxylic acid (2.0 g, 8.1 mmol) in DCM (20 mL) was added oxalic dichloride (1.34 g, 10.5 mmol) and DMF (30 mg, 0.4 mmol). The resulting solution was stirred at room temperature for 1 h. Et₃N (3.2 g, 3.2 mmol) and (2S)-3-methyl-2-(methylamino)butanoic acid (1.25 g, 9.5 mmol) were added and the mixture was stirred at room temperature for 1 h. H₂O (100 mL) was added and the mixture was extracted with EtOAc (50 mL×3). The combined organic layers were concentrated under reduced pressure and the residue was purified by silica gel column chromatography to give tert-butyl (S)-4-((1-(tert-butoxy)-3-methyl-1-oxobutan-2-yl)(methyl)carbamoyl)-4-fluoropiperidine-1-carboxylate (1.34 g, 45% yield) as a solid. LCMS (ESI): m/z [M+Na] calc'd for C₂₁H₃₇FN₂O₅Na 439.3; found 439.3.

Step 2. A mixture of tert-butyl (S)-4-((1-(tert-butoxy)-3-methyl-1-oxobutan-2-yl)(methyl)carbamoyl)-4-fluoropiperidine-1-carboxylate (290 mg, 0.70 mmol) in DCM (4 mL) and TFA (2 mL) was stirred at room temperature for 2 h, then concentrated under reduced pressure to give N-(4-fluoropiperidine-4-carbonyl)-N-methyl-L-valine, which was used directly in the next step without further purification. LCMS (ESI): m/z: [M+H] calc'd for C₁₂H₂₁FN₂O₃ 260.2; found 261.2.

Step 3. To a solution of the tert-butyl N-(4-fluoropiperidine-4-carbonyl)-N-methyl-L-valinate (1.7 g, 5.3 mmol), sodium 4-(dimethylamino)-4-methylpent-2-ynoate (1.67 g, 9.4 mmol) and Et₃N (2.73 g, 36.9 mmol) in DMF (20 mL) stirred at 5° C. was added T3P (4.11 g, 10.7 mmol, 50 wt % in EtOAc). The reaction mixture was stirred at 5° C. for 1 h. The resulting mixture was quenched with H₂O (100 mL) and extracted with EtOAc (50 mL×3). The combined organic layers were concentrated and purified by silica gel column chromatography to give tert-butyl N-(1-(4-(dimethylamino)-4-methylpent-2-ynoyl)-4-fluoropiperidine-4-carbonyl)-N-methyl-L-valinate (1.6 g, 74.0% yield) as a solid. LCMS (ESI): m/z: [M+H] calc'd for C₂₄H₄₀FN₃O₄ 453.3; found 454.2.

Step 4. To a solution of tert-butyl N-(1-(4-(dimethylamino)-4-methylpent-2-ynoyl)-4-fluoropiperidine-4-carbonyl)-N-methyl-L-valinate (50 mg, 0.11 mmol) in DCM (2 mL) was added TFA (1 mL). The reaction mixture was stirred at 20° C. for 2 h, then concentrated under reduced pressure to afford crude N-(1-(4-(dimethylamino)-4-methylpent-2-ynoyl)-4-fluoropiperidine-4-carbonyl)-N-methyl-L-valine. It was used for the next step directly without further purification. LCMS (ESI): m/z: [M+H] calc'd for C₂₀H₃₂FN₃O₄ 397.2; found 398.3.

Step 5. To a solution of tert-butyl (2R)-2-(hydroxymethyl)morpholin-4-yl formate (50 g, 230 mmol) in EtOAc (1 L) was added TEMPO (715 mg, 4.6 mmol) and NaHCO₃ (58 g, 690 mmol) at 20° C. The mixture was cooled to −50° C., then TCCA (56 g, 241 mmol) in EtOAc (100 mL) was added dropwise over 30 min. The reaction mixture was warmed to 5° C. for 2 h, then quenched with 10% Na₂S₂O₃ (200 mL) and stirred for 20 min. The resulting mixture was filtered and the organic phase was separated from filtrate. The aqueous phase was extracted with EtOAc (100 mL×2). The combined organic layers were washed with H₂O (100 mL) and brine (100 mL), and dried over anhydrous Na₂SO₄. The organic layer was concentrated under reduced pressure to afford tert-butyl (2R)-2-formylmorpholin-4-yl formate (50 g, crude) as an oil.

Step 6. To a solution of tert-butyl (2R)-2-formylmorpholin-4-yl formate (49 g, 153 mmol) and methyl 2-{[(benzyloxy)carbonyl]amino}-2-(dimethoxyphosphoryl)acetate (60 g, 183 mmol) in CAN (300 mL) was added tetramethylguanidine (35 g, 306 mmol) at 0-10° C. The reaction mixture was stirred at 10° C. for 30 min then warmed to 20° C. for 2 h. The reaction mixture was diluted with DCM (200 mL) and washed with Citric acid (10%, 200 mL) and 10% NaHCO₃ aqueous solution (200 mL). The organic phase was concentrated under reduced pressure, and purified by silica gel column chromatography to afford tert-butyl (S,Z)-2-(2-(((benzyloxy)carbonyl)amino)-3-methoxy-3-oxoprop-1-en-1-yl)morpholine-4-carboxylate (36 g, 90% yield) as solid. LCMS (ESI): m/z: [M+Na] calc'd for C₂₁H₂₈N₂O₄ 420.2; found: 443.1.

Step 7. To a solution of tert-butyl (S,Z)-2-(2-(((benzyloxy)carbonyl)amino)-3-methoxy-3-oxoprop-1-en-1-yl)morpholine-4-carboxylate (49 g, 0.12 mol) in MeOH (500 mL) was added (S,S)-Et-DUPHOS-Rh (500 mg, 0.7 mmol). The mixture was stirred at 25° C. under an H₂ (60 psi) atmosphere for 48 h. The reaction was concentrated and purified by chromatography to give tert-butyl (S)-2-((S)-2-(((benzyloxy)carbonyl)amino)-3-methoxy-3-oxopropyl)morpholine-4-carboxylate (44 g, 89.8% yield) as solid. LCMS (ESI): m/z: [M+Na] calc'd for C₂₁H₃₀N₂O₇ 422.2; found: 445.2.

Step 8. To a stirred solution of tert-butyl (S)-2-((S)-2-(((benzyloxy)carbonyl)amino)-3-methoxy-3-oxopropyl)morpholine-4-carboxylate (2.2 g, 5.2 mmol) in EtOAc (2 mL) was added HCl/EtOAc (25 mL) at 15° C. The reaction was stirred at 15° C. for 2 h, then concentrated under reduced pressure to afford methyl (S)-2-(((benzyloxy)carbonyl)amino)-3-((S)-morpholin-2-yl)propanoate (1.51 g, 90.4% yield) as an oil. LCMS (ESI): m/z: [M+H] calc'd for C₁₆H₂₂N₂O₅ 322.1; found 323.2.

Step 9. To a solution of 3-(5-bromo-1-ethyl-2-{2-[(1S)-1-methoxyethyl]pyridin-3-yl}indol-3-yl)-2,2-dimethylpropan-1-ol (100 g, 0.22 mol) and 1H-imidazole (30.6 g, 0.45 mol) in DCM (800 mL) was added TBSCI (50.7 g, 0.34 mol) in DCM (200 mL) at 0° C. The reaction was stirred at 25° C. for 2 h. The resulting solution was washed with H₂O (300 mL×3) and brine (200 mL×2), dried over anhydrous sodium sulfate, filtered and concentrated under reduced pressure. The residue was purified with silica gel column chromatography to give (S)-5-bromo-3-(3-((tert-butyldimethylsilyl)oxy)-2,2-dimethylpropyl)-1-ethyl-2-(2-(1-methoxyethyl)pyridin-3-yl)-1H-indole (138 g, 90% yield) as an solid. LCMS (ESI): m/z: [M+H] calc'd for C₂₉H₄₃BrN₂O₂Si 558.2; found 559.2.

Step 10. To a stirred solution of Intermediate 1 (50 g, 89.3 mmol) in dioxane (500 mL) was added methyl (2S)-2-{[(benzyloxy)carbonyl]amino}-3-[(2S)-morpholin-2-yl]propanoate from step 1 (31.7 g, 98.2 mmol), RuPhos (16.7 g, 35.7 mmol), Di-mu-chlorobis(2-amino-1,1-biphenyl-2-yl-C,N)dipalladium(II) (2.8 g, 4.4 mmol) and cesium carbonate (96 g, 295 mmol) followed by RuPhos-Pd-G2 (3.5 g, 4.4 mmol) at 105° C. under an N₂ atmosphere. The reaction mixture was stirred for 6 h at 105° C. under an N₂ atmosphere. The resulting mixture was filtered, and the filtrate was concentrated under reduced pressure. The residue was purified by prep-TLC chromatography to afford methyl (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl)morpholin-2-yl]propanoate (55 g, 73% yield) as a solid. LCMS (ESI): m/z: [M+H] calc'd for C₄₅H₆₄N₄O₇Si 800.5; found 801.5.

Step 11. To a solution of methyl (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl)morpholin-2-yl]propanoate (10 g, 12 mmol) in THF (270 mL) was added LiOH (1.3 g, 31 mmol) in H₂O (45 mL) at 20° C. The reaction was stirred at 20° C. for 2 h, then treated with 1N HCl to adjust pH to 4˜5 at 0˜5° C. The resulting mixture was extracted with EtOAc (50 mL×2). The combined organic layers were washed with brine and dried over anhydrous Na₂SO₄. The organic phase was then concentrated under reduced pressure to afford (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl)morpholin-2-yl]propanoic acid (9.5 g, 97% yield) as a solid. LCMS (ESI): m/z: [M+H] calc'd for C₄₄H₆₂N₄O₇Si 786.4; found 787.4.

Step 12. To a stirred solution of (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl)morpholin-2-yl]propanoic acid (10 g, 12.7 mmol) in DMF (150 mL), was added methyl (S)-hexahydropyridazine-3-carboxylate (2 g, 14 mmol), then cooled to 0° C., DIPEA (32.8 g, 254 mmol) was added followed by HATU (9.7 g, 25.4 mmol) at 0-5° C. The reaction mixture was stirred at 0-5° C. for 1 h. The resulting mixture was diluted with EtOAc (500 mL) and H₂O (200 mL). The organic layer was separated and washed with H₂O (100 mL×2) and brine (100 mL), dried over anhydrous sodium sulfate. The solution was filtered and concentrated under reduced pressure, and the residue was purified by silica gel column chromatography to afford methyl (S)-1-((S)-2-(((benzyloxy)carbonyl)amino)-3-((S)-4-(3-(3-((tert-butyldimethylsilyl)oxy)-2,2-dimethylpropyl)-1-ethyl-2-(2-((S)-1-methoxyethyl)pyridin-3-yl)-1H/indol-5-yl)morpholin-2-yl)propanoyl)hexahydropyridazine-3-carboxylate (8 g, 70% yield) as a solid. LCMS (ESI): m/z: [M+H] calc'd for C₅₀H₇₂N₆O₈Si 912.5; found 913.4.

Step 13. A solution of methyl (S)-1-((S)-2-(((benzyloxy)carbonyl)amino)-3-((S)-4-(3-(3-((tert-butyldimethylsilyl)oxy)-2,2-dimethylpropyl)-1-ethyl-2-(2-((S)-1-methoxyethyl)pyridin-3-yl)-1H-indol-5-yl)morpholin-2-yl)propanoyl)hexahydropyridazine-3-carboxylate (8.5 g, 9 mmol) in THF (8 mL) was added a mixture of tetrabutylammonium fluoride (1M in THF, 180 mL, 180 mmol) and AcOH (11 g, 200 mmol) at 20° C. The reaction mixture was stirred at 75° C. for 3 h. The resulting mixture was diluted with EtOAc (150 mL) and washed with H₂O (20 mL×6). The organic phase was concentrated under reduced pressure to give methyl (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylate (7.4 g, 100% yield) as solid. LCMS (ESI): m/z: [M+H] calc'd for C₄₄H₅₈N₆O₈ 799.4; found 798.4.

Step 14. To a solution of methyl (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylate (8 g, 10 mmol) in THF (200 mL) was added lithium hydroxide (600 mg, 25 mmol) in H₂O (30 mL). The reaction mixture was stirred at 20° C. for 1 h, then treated with 1N HCl to adjust pH to 4˜5 at 0˜5° C., and extracted with EtOAc (500 mL×2). The organic phase was washed with brine, and concentrated under reduced pressure to afford (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylic acid (8 g, crude) as a solid. LCMS (ESI): m/z: [M+H] calc'd for C₄₃H₅₆N₆O₈ 784.4; found 785.4.

Step 15. To a stirred solution of (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylic acid (8 g, 10.2 mmol) and DIPEA (59 g, 459 mmol) in DCM (800 mL) was added EDCI (88 g, 458 mmol) and HOBT (27.6 g, 204 mmol) at 25° C. under an argon atmosphere. The reaction mixture was stirred at 25° C. for 16 h. The resulting mixture was concentrated under reduced pressure, and the residue was purified by silica gel column chromatography to afford benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (5 g, 66% yield) as a solid; LCMS (ESI): m/z: [M+H] calc'd for C₄₃H₅₄N₆O₇ 766.4; found 767.4.

Step 16. To a solution of benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)carbamate (400 mg, 0.5 mmol) in MeOH (20 mL) was added Pd/C (200 mg) and ammonium acetate (834 mg, 16 mmol) at 20° C. under an H₂ atmosphere and the mixture was stirred for 2 h. Then resulting mixture was filtered and concentrated under reduced pressure. The residue was redissolved in DCM (20 mL) and washed with H₂O (5 mL×2), then concentrated under reduced pressure to afford (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (320 mg, 97% yield) as a solid. LCMS (ESI): m/z: [M+H] calc'd for C₃₅H₄₆N₆O₅ 632.4; found 633.3.

Step 17. To a solution of the (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (50 mg, 0.079 mmol), N-(1-(4-(dimethylamino)-4-methylpent-2-ynoyl)-4-fluoropiperidine-4-carbonyl)-N-methyl-L-valine (47 mg, 0.12 mmol) in DMF (2 mL) stirred at 0° C. was added HATU (36 mg, 0.09 mmol) and DIPEA (153 mg, 1.2 mmol) dropwise. The reaction was stirred at 0° C. for 1 h. The resulting mixture was purified by reverse phase to afford 1-(4-(dimethylamino)-4-methylpent-2-ynoyl)-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-4-fluoro-N-methylpiperidine-4-carboxamide (11.9 mg, 13.9% yield) as a solid. ¹H NMR (400 MHz, CD₃OD) δ 8.71 (dd, J=4.8, 1.7 Hz, 1H), 7.86 (d, J=7.8 Hz, 1H), 7.51 (dd, J=7.8, 4.8 Hz, 1H), 7.39 (d, J=8.9 Hz, 1H), 7.15-7.04 (m, 2H), 5.67 (d, J=8.8 Hz, 1H), 4.62 (d, J=11.2 Hz, 1H), 4.46 (d, J=12.4 Hz, 1H), 4.39-4.27 (m, 2H), 4.23 (d, J=6.1 Hz, 1H), 4.17-4.08 (m, 1H), 3.93 (s, 2H), 3.86 (s, 1H), 3.84-3.76 (m, 2H), 3.74-3.65 (m, 2H), 3.63-3.51 (m, 2H), 3.27-3.23 (m, 1H), 3.22-3.11 (m, 6H), 3.0-2.89 (m, 2H), 2.85-2.75 (m, 2H), 2.74-2.55 (m, 2H), 2.36 (d, J=8.2 Hz, 6H), 2.32-2.21 (m, 2H), 2.20-2.02 (m, 5H), 1.92 (d, J=12.5 Hz, 2H), 1.69 (dd, J=43.8, 12.6 Hz, 2H), 1.46 (dt, J=8.0, 4.9 Hz, 9H), 1.03 (d, J=3.5 Hz, 3H), 0.90 (dd, J=48.3, 6.5 Hz, 6H), 0.77 (d, J=3.0 Hz, 3H), 0.69 (s, 3H). LCMS (ESI): m/z: [M+H] calc'd for C₅₅H₇₈FN₉O₈ 1011.6; found 1012.5.

### Example A375. Synthesis of (2R)-2-(((1-(4-(dimethylamino)-4-methylpent-2-ynoyl)azetidin-3-yl)oxy)methyl)N-((2²S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)-3-methylbutanamide

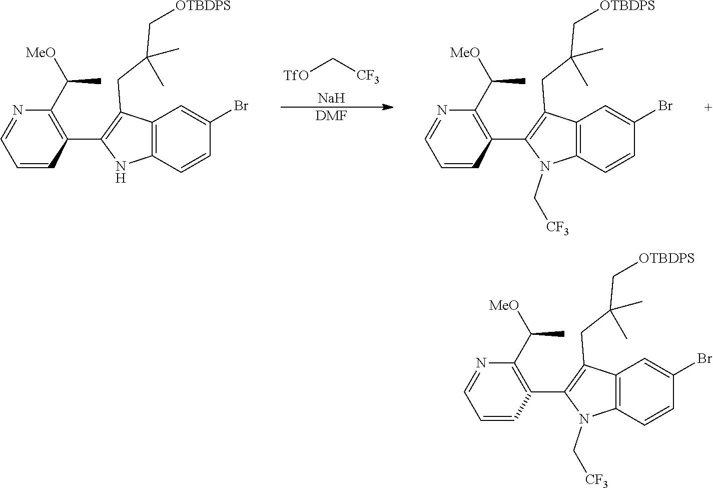

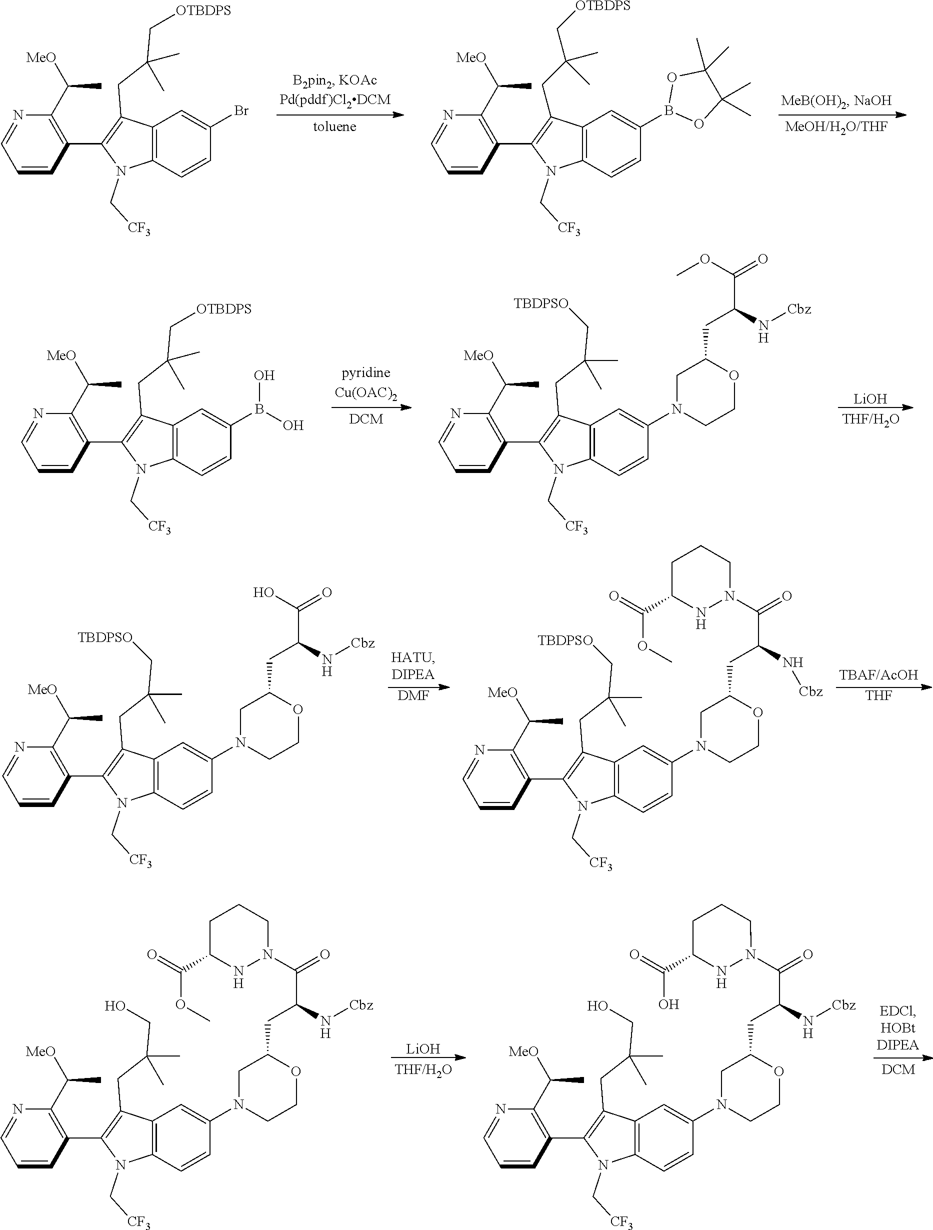

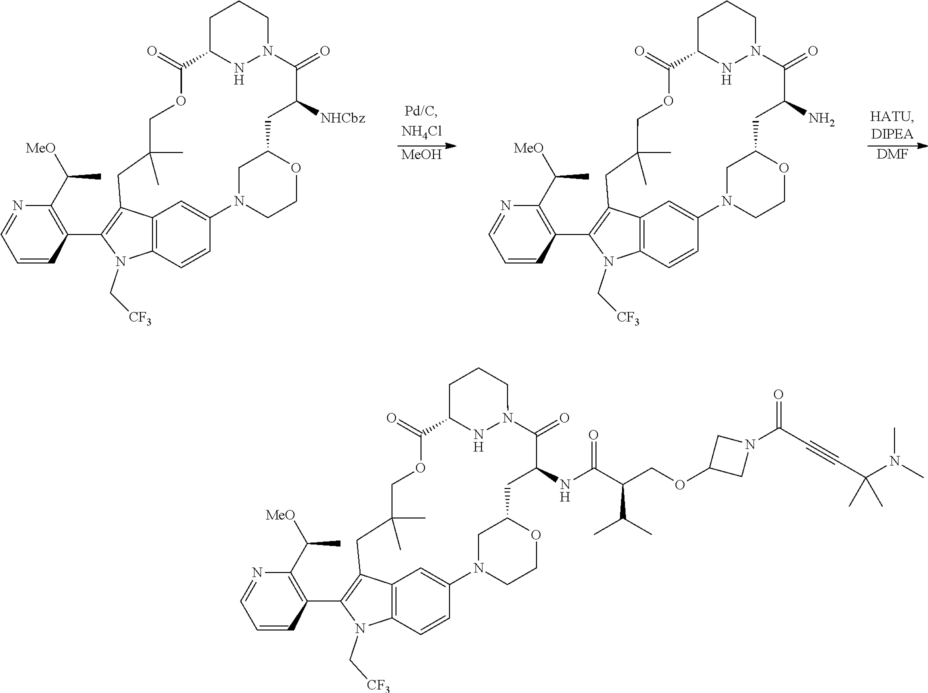

Step 1. To a mixture of 5-bromo-3-{3-[(tert-butyldiphenylsilyl)oxy]-2,2-dimethylpropyl}-2-{2-[(1S)-1-methoxyethyl]pyridin-3-yl}-1H-indole (10.0 g, 15.2 mmol) in anhydrous DMF (120 ml) at 0° C. under an atmosphere of N₂ was added NaH, 60% dispersion in oil (1.2 g, 30.4 mmol) and 2,2,2-trifluoroethyl trifluoromethanesulfonate (35.4 g, 152 mmol). The mixture was stirred at 0° C. for 1 h, then saturated NH₄Cl (30 ml) added and the mixture extracted with EtOAc (100 ml×3). The combined organic layers were washed with brine, dried over anhydrous Na₂SO₄ and filtered. The filtrate was concentrated under reduced pressure and the residue was purified by silica gel column chromatography to give (S)-5-bromo-3-(3-((tert-butyldiphenylsilyl)oxy)-2,2-dimethylpropyl)-2-(2-(1-methoxyethyl)pyridin-3-yl))-1-(2,2,2-trifluoroethyl)-1H-indole (8 g) as an oil and the other atropisomer (6 g, 48% yield) as an oil. LCMS (ESI): m/z: [M+H] calc'd for C₃₉H₄BrF₃N₂O₂Si 73.2; found 737.1.

Step 2. To a mixture of (S)-5-bromo-3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indole (7.2 g, 9.7 mmol) in toluene (80 mL) under an atmosphere of N₂ was added 4,4,4′,4′,5,5,5′,5′-octamethyl-2,2′-bi(1,3,2-dioxaborolane) (2.7 g, 10.6 mmol), KOAc (1.9 g, 19.4 mmol) and Pd(dppf)Cl₂ DCM (0.8 g, 0.1 mmol). The mixture was heated to 90° C. and stirred for 8 h, then saturated NH₄Cl (30 mL) added and the mixture extracted with EtOAc (40 mL×3). The combined organic layers were dried over anhydrous Na₂SO₄ and filtered. The filtrate was concentrated under reduced pressure and the residue was purified by silica gel column chromatography to give (S)-3-(3-((tert-butyldiphenylsilyl)oxy)-2,2-dimethylpropyl)-2-(2-(1-methoxyethyl)pyridin-3-yl)-5-(4,4,5,5-tetramethyl-1,3,2-dioxaborolan-2-yl)-1-(2,2,2-trifluoroethyl)-1H-indole (6.1 g, 64% yield) as an oil. LCMS (ESI): m/z: [M+H] calc'd for C₄₅H₅₆BF₃N₂O₄Si 784.4; found 785.3.

Step 3. To a mixture of (S)-3-(3-((tert-butyldiphenylsilyl)oxy)-2,2-dimethylpropyl)-2-(2-(1-methoxyethyl)pyridin-3-yl)-5-(4,4,5,5-tetramethyl-1,3,2-dioxaborolan-2-yl)-1-(2,2,2-trifluoroethyl)-1H-indole (33 g, 42 mmol) in THF (120 mL) and MeOH (330 mL) at 0° C. under an atmosphere of N₂ was added MeB(OH)₂ (50.4 g, 841 mmol), then a mixture of NaOH (33.6 g, 841 mmol) in H₂O (120 mL). The mixture was warmed to room temperature and stirred for 16 h, then concentrated under reduced pressure. H₂O (500 mL) was added to the residue and the mixture extracted with EtOAc (300 mL×3). The combined organic layers were washed with brine (300 mL), H₂O (300 mL), then concentrated under reduced pressure and the residue was purified by silica gel column chromatography to give (S)-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)boronic acid (20 g, 68% yield) as a solid. LCMS (ESI): m/z: [M+H] calc'd for C₃₉H₄₆BF₃N₂O₄Si 702.3; found 703.3.

Step 4. Note: Three reactions were run in parallel—the yield reflects the sum of the products.

A mixture of (S)-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)boronic acid (1.85 g, 5.6 mmol) and methyl (S)-2-(((benzyloxy)carbonyl)amino)-3-((S)-morpholin-2-yl)propanoate in DCM (150 mL) under air was added pyridine (1.35 g, 16.9 mmol) and Cu(OAc)₂ (2.0 g, 11.3 mmol). The mixture was stirred for 48 h, then concentrated under reduced pressure. H₂O (300 mL) was added to the residue and the mixture was extracted with EtOAc (300 mL×2). The combined organic layers were washed with brine (100 mL), dried over anhydrous Na₂SO₄ and filtered. The filtrate was concentrated under reduced pressure and the residue was silica gel column chromatography to give methyl (S)-2-(((benzyloxy)carbonyl)amino)-3-((S)-4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)morpholin-2-yl)propanoate (9.2 g, 55% yield) as a solid. LCMS (ESI): m/z [M/2+H] calc'd for C₅₅H₆₅F₃N₄O₇Si 490.2; found 490.3.

Step 5. To a mixture of methyl (S)-2-(((benzyloxy)carbonyl)amino)-3-((S)-4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)morpholin-2-yl)propanoate (10.8 g, 11.0 mmol) in THF (50 mL) was added LiOH (528 mg, 22 mmol) in H₂O (10 mL). The mixture was stirred for 1 h, then cooled to 0-5° C. and acidified to pH˜7 using 2N HCl (10 mL). The mixture was extracted with DCM (100 mL×2) and the combined organic layers were dried over Na₂SO₄ and filtered. The filtrate was concentrated under reduced pressure to give (S)-2-(((benzyloxy)carbonyl)amino)-3-((S)-4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)morpholin-2-yl)propanoic acid (10.6 g, 100% yield) as a solid. LCMS (ESI): m/z: [M/2+H] calc'd for C₅₄H₆₃F₃N₄O₇Si 483.2; found 483.3.

Step 6. To a mixture of (S)-2-(((benzyloxy)carbonyl)amino)-3-((S)-4-(3-(3-((tert-butyldiphenylsilyl)oxy)-2,2-dimethylpropyl)-2-(2-((5)-1-methoxyethyl)pyridin-3-yl)-1-(2,2,2-trifluoroethyl)-1H-indol-5-yl)morpholin-2-yl)propanoic acid (10.6 g, 11.0 mmol) and methyl (3S)-1,2-diazinane-3-carboxylate (15.8 g, 22.0 mmol) in DMF (150 mL) at 0° C. was added DIPEA (28.4 g, 220 mmol) and HATU (8.4 g, 22.0 mmol). The mixture was stirred at 0-5° C. for 1 h, then EtOAc (500 mL) was added and the mixture was washed with H₂O (200 mL×2), brine (100 mL), dried over anhydrous Na₂SO₄ and filtered. The filtrate was concentrated under reduced pressure and the residue was purified by silica gel column chromatography to give methyl (S)-1-((5)-2-(((benzyloxy)carbonyl)amino)-3-((S)-4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)morpholin-2-yl)propanoyl)hexahydropyridazine-3-carboxylate (11 g, 90% yield) as a solid. LCMS (ESI): m/z: [M/2+H] calc'd for C₆₀H₇₃F₃N₆O₈Si 546.3; found 546.3.

Step 7. To a mixture of methyl (S)-1-((S)-2-(((benzyloxy)carbonyl)amino)-3-((S)-4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)morpholin-2-yl)propanoyl)hexahydropyridazine-3-carboxylate (11.0 g, 10.1 mmol) in THF (10 mL) was added a mixture of AcOH (21.2 g, 353 mmol) and 1M TBAF in THF (300 mL, 300 mmol). The mixture was heated to 80° C. and stirred for 16 h, then concentrated under reduced pressure. EtOAc (800 mL) was added to the residue and the mixture was washed with H₂O (80 mL×6), concentrated under reduced pressure and the residue was purified by preparative-HPLC to give methyl (S)-1-((S)-2-(((benzyloxy)carbonyl)amino)-3-((S)-4-(3-(3-hydroxy-2,2-dimethylpropyl)-2-(2-((S)-1-methoxyethyl)pyridin-3-yl)-1-(2,2,2-trifluoroethyl)-1H-indol-5-yl)morpholin-2-yl)propanoyl)hexahydropyridazine-3-carboxylate (7.9 g, 91% yield) as a solid. LCMS (ESI): m/z: [M+H] calc'd for C₄₄H₅₅F₃N₆O₈ 852.4; found 853.3.

Step 8. To a mixture of methyl (S)-1-((S)-2-(((benzyloxy)carbonyl)amino)-3-((S)-4-(3-(3-hydroxy-2,2-dimethylpropyl)-2-(2-((S)-1-methoxyethyl)pyridin-3-yl)-1-(2,2,2-trifluoroethyl)-1H-indol-5-yl)morpholin-2-yl)propanoyl)hexahydropyridazine-3-carboxylate (7.9 g, 9.3 mmol) in THF (50 mL) was added LiOH (443 mg, 18.5 mmol) in H₂O (10 mL). The mixture was stirred for 1 h, then cooled to 0-5° C. and acidified to ˜pH 7 with 2N HCl (9 mL). The mixture was extracted with DCM (100 mL×2) and the combined organic layers were dried over Na₂SO₄ and filtered. The filtrate was concentrated under reduced pressure to give (S)-1-((S)-2-(((benzyloxy)carbonyl)amino)-3-((S)-4-(3-(3-hydroxy-2,2-dimethylpropyl)-2-(2-((S)-1-methoxyethyl)pyridin-3-yl)-1-(2,2,2-trifluoroethyl)-1H-indol-5-yl)morpholin-2-yl)propanoyl)hexahydropyridazine-3-carboxylic acid (7.6 g, 98% yield) as a solid. LCMS (ESI): m/z: [M+H] calc'd for C₄₃H₅₃F₃N₆O₈ 838.4; found 839.3.

Step 9. To a mixture of (S)-1-((S)-2-(((benzyloxy)carbonyl)amino)-3-((S)-4-(3-(3-hydroxy-2,2-dimethylpropyl)-2-(2-((S)-1-methoxyethyl)pyridin-3-yl)-1-(2,2,2-trifluoroethyl)-1H-indol-5-yl)morpholin-2-yl)propanoyl)hexahydropyridazine-3-carboxylic acid (7.6 g, 9.0 mmol) and DIPEA (52.3 g, 405 mmol) in DCM (800 mL) under an atmosphere of Ar was added EDCI (77.6 g, 405 mmol) and HOBT (12 g, 90 mmol). The mixture was stirred for 16 h, then concentrated under reduced pressure. The residue was diluted with EtOAc (500 mL), washed with H₂O (100 mL×2) and filtered. The organic layer was concentrated under reduced pressure and the residue was purified by silica gel column chromatography to give benzyl ((2²S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (6.1 g, 74% yield) as a solid. LCMS (ESI): m/z: [M+H] calc'd for C₄₃H₅₁F₃N₆O₇ 820.4; found 821.3.

Step 10. To a mixture of benzyl ((2²S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (700 mg, 0.85 mmol) in MeOH (30 mL) was added 10% Pd on C (317 mg) and NH₄Cl (909 mg). The mixture was stirred under an atmosphere of H₂ (1 atm) for 16 h, then filtered through Celite and the filter cake was washed with MeOH (150 mL). The filtrate was concentrated under reduced pressure, DCM (20 mL) was added to the residue and the mixture was washed with saturated NaHCO₃ (20 mL×3). The organic layer was concentrated under reduced pressure to give (2²S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)- pyridazinacycloundecaphane-5,7-dione (660 mg, 95% yield) as a solid. LCMS (ESI): m/z: [M+H] calc'd for C₃₅H₄₅F₃N₆O₅ 686.3; found 687.3; ¹H NMR (400 MHz, CDCl₃) δ 8.80 (dd, J=4.7, 1.7 Hz, 1H), 7.66 (d, J=7.4 Hz, 1H), 7.43-7.30 (m, 2H), 7.12-7.01 (m, 2H), 4.90-4.83 (m, 1H), 4.68 (d, J=12.5 Hz, 1H), 4.57 (dd, J=16.2, 8.1 Hz, 1H), 4.24 (q, J=6.1 Hz, 1H), 4.08 (d, J=10.6 Hz, 1H), 3.97-3.82 (m, 4H), 3.80-3.68 (m, 2H), 3.55 (d, J=11.6 Hz, 1H), 3.21 (d, J=9.4 Hz, 1H), 2.93 (dd, J=19.9, 9.3 Hz, 3H), 2.66 (t, J=11.6 Hz, 1H), 2.47 (d, J=14.5 Hz, 1H), 2.19-2.04 (m, 4H), 1.96 (d, J=13.6 Hz, 2H), 1.80-1.71 (m, 2H), 1.66-1.59 (m, 1H), 1.47 (d, J=6.1 Hz, 3H), 0.88 (s, 3H), 0.42 (s, 3H).

Step 11. To a mixture of (2²S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (300 mg, 0.4 mmol), (2R)-2-[({1-[4-(dimethylamino)-4-methylpent-2-ynoyl]azetidin-3-yl}oxy)methyl]-3-methylbutanoic acid (157 mg, 0.48 mmol) and DIPEA (569.0 mg, 0.4 mmol) in DMF (5 mL) at 0° C. was added HATU (217 mg, 0.57 mmol). The mixture was stirred at 0° C. for 0.5 h, then diluted with H₂O and extracted with EtOAc (20 mL×3). The combined organic layers were washed with brine (20 mL×3), dried over Na₂SO₄ and filtered. The filtrate was concentrated under reduced pressure and the residue was purified by preparative-TLC to give (2R)-2-(((1-(4-(dimethylamino)-4-methylpent-2-ynoyl)azetidin-3-yl)oxy)methyl)-N-((2²S,6³S,4S)-12-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide (200 mg, 46% yield) as a solid. LCMS (ESI): m/z: [M+H] calc'd for C₅₂H₇₁F₃N₈O₈ 992.5; found 993.4; ¹H NMR (400 MHz, CD₃OD) δ 8.74 (m, 1H), 8.00 (m, 1H), 7.85 (d, J=7.7 Hz, 1H), 7.60-7.43 (m, 2H), 7.14 (t, J=9.5 Hz, 2H), 5.63 (s, 1H), 5.06 (m, 1H), 4.64 (s, 1H), 4.52-4.31 (m, 3H), 4.27-4.05 (m, 3H), 3.97 (m, 1H), 3.92-3.66 (m, 6H), 3.59 (m, 2H), 3.46 (m, 2H), 3.25 (d, J=5.3 Hz, 3H), 3.07-2.89 (m, 2H), 2.86-2.59 (m, 3H), 2.38-2.32 (m, 3H), 2.28 (s, 3H), 2.13 (m, 2H), 2.03-1.51 (m, 6H), 1.50-1.41 (m, 6H), 1.38 (d, J=7.5 Hz, 3H), 0.98 (t, J=8.7 Hz, 6H), 0.89 (t, J=6.4 Hz, 3H), 0.54 (d, J=8.4 Hz, 3H).

### Example A722. Synthesis of 1-acryloyl-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-4-fluoro-N-methylpiperidine-4-carboxamide

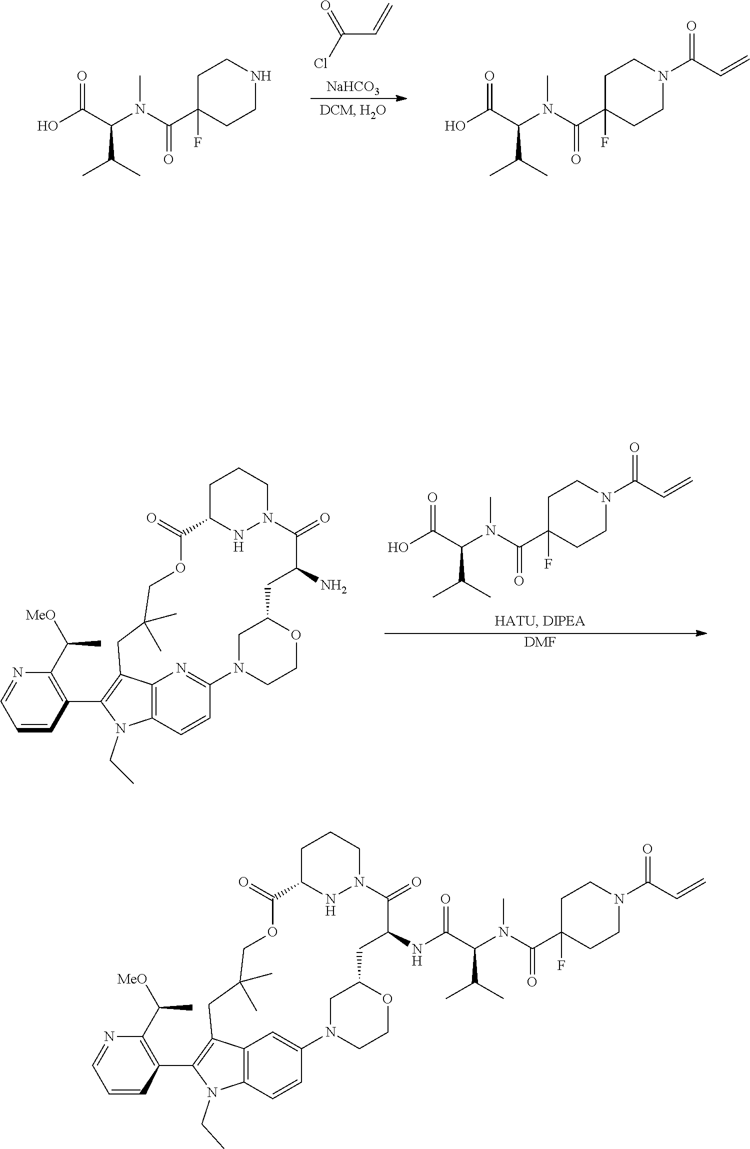

Step 1. To a mixture of N-(4-fluoropiperidine-4-carbonyl)-N-methyl-L-valine (190 mg, 0.73 mmol) and NaHCO₃ (306 mg, 3.6 mmol) in DCM (2 mL) and H₂O (1 mL) at −10° C. was added prop-2-enoyl chloride (132 mg, 1.45 mmol). The mixture was stirred at 0-5° C. for 1 h, then diluted with DCM (20 mL) and washed with H₂ (20 mL×2), brine (20 mL) and the organic layer dried over Na₂SO₄ and filtered. The filtrate was concentrated under reduced pressure and the residue was purified by silica gel column chromatography to give N-(1-acryloyl-4-fluoropiperidine-4-carbonyl)-N-methyl-L-valine (120 mg, 52% yield) as a solid. LCMS (ESI): m/z: [M+H] calc'd for C₁₅H₂₃FN₂O₄ 314.2; found 315.2.

Step 2. To a mixture of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (153 mg, 0.24 mmol), N-(1-acryloyl-4-fluoropiperidine-4-carbonyl)-N-methyl-L-valine (106 mg, 0.34 mmol) in DMF (2 mL) at 5° C. was added HATU (110 mg, 0.29 mmol) and DIPEA (468 mg, 3.6 mmol) dropwise. The mixture was stirred at 5° C. for 1 h, then purified by preparative-HPLC to give 1-acryloyl-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)amino)-3-methyl-1-oxobutan-2-yl)-4-fluoro-N-methylpiperidine-4-carboxamide (69.5 mg, 29% yield) as a solid. LCMS (ESI): m/z: [M+H] calc'd for C₅₀H₆₉FN₈O₈ 928.5; found 929.4; ¹H NMR (400 MHz, CD₃OD) δ 8.71 (d, J=3.2 Hz, 1H), 7.86 (d, J=7.7 Hz, 1H), 7.51 (dd, J=7.7, 4.8 Hz, 1H), 7.39 (d, J=8.9 Hz, 1H), 7.18-7.02 (m, 2H), 6.80 (dd, J=16.8, 10.7 Hz, 1H), 6.23 (d, J=16.8 Hz, 1H), 5.77 (d, J=10.6 Hz, 1H), 5.67 (d, J=6.5 Hz, 1H), 4.61 (d, J=11.1 Hz, 1H), 4.44 (t, J=15.1 Hz, 2H), 4.23 (q, J=6.1 Hz, 1H), 4.18-4.10 (m, 1H), 4.09-4.01 (m, 1H), 3.99-3.83 (m, 3H), 3.83-3.65 (m, 4H), 3.58-3.46 (m, 2H), 3.27 (s, 1H), 3.21-3.11 (m, 6H), 3.00-2.91 (m, 2H), 2.85-2.75 (m, 2H), 2.73-2.64 (m, 1H), 2.62-2.54 (m, 1H), 2.36-2.21 (m, 2H), 2.19-2.01 (m, 5H), 1.92 (d, J=12.7 Hz, 2H), 1.79-1.57 (m, 2H), 1.44 (d, J=6.2 Hz, 3H), 1.04 (t, J=6.3 Hz, 3H), 0.98-0.81 (m, 6H), 0.76 (s, 3H), 0.68 (s, 3H).

### Example A377. Synthesis of (2R)-2-(((1-(4-(dimethylamino)-4-methylpent-2-ynoyl)azetidin-3-yl)oxy)methyl)N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide

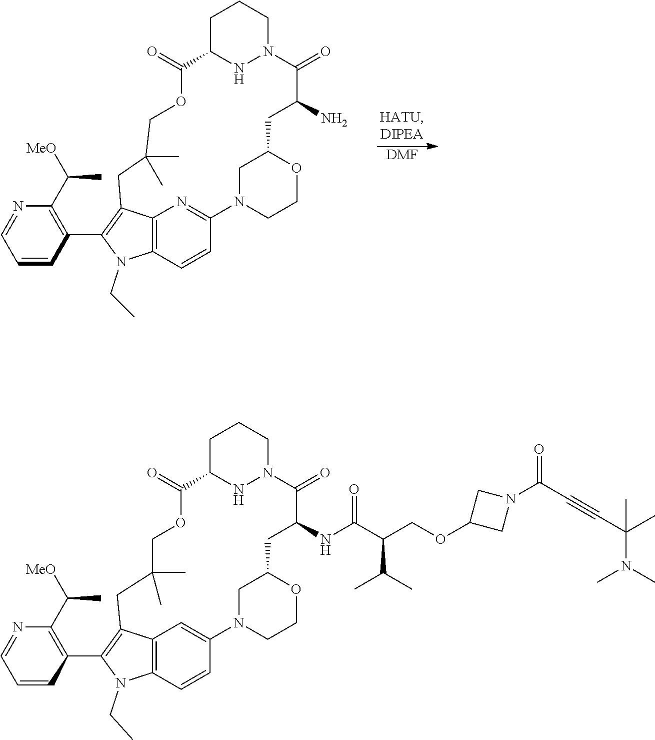

Step 1. (2R)-2-(((1-(4-(dimethylamino)-4-methylpent-2-ynoyl)azetidin-3-yl)oxy)methyl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide was synthesized in a manner similar to (2R)-2-(((1-(4-(dimethylamino)-4-methylpent-2-ynoyl)azetidin-3-yl)oxy)methyl)-N-((2²S,6³S,4S)-12-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide except (2²S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione was substituted with (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione and 3-((1-[4-(dimethylamino)-4-methylpent-2-ynoyl]azetidin-3-yl)oxy)propanoic acid was substituted with (2R)-2-[({1-[4-(dimethylamino)-4-methylpent-2-ynoyl]azetidin-3-yl}oxy)methyl]-3-methylbutanoic acid to give the desired product (25.6 mg, 26% yield) as a solid. LCMS (ESI): m/z: [M+H] calc'd for C₅₂H₇₄N₈O₈ 938.6; found 939.5; ¹H NMR (400 MHz, CD₃OD) δ 8.72 (dd, J=4.8, 1.5 Hz, 1H), 7.97 (dd, J=20.0, 6.8 Hz, 1H), 7.87 (dd, J=5.8, 2.6 Hz, 1H), 7.54-7.51 (m, 1H), 7.41 (d, J=8.9 Hz, 1H), 7.14 (dd, J=36.0, 10.4 Hz, 2H), 5.65 (s, 1H), 4.49-4.33 (m, 3H), 4.27-4.08 (m, 4H), 3.96 (d, J=8.6 Hz, 2H), 3.87 (dd, J=10.8, 3.6 Hz, 2H), 3.79 (dd, J=10.8, 7.7 Hz, 3H), 3.69-3.58 (m, 3H), 3.43 (dd, J=23.9, 11.7 Hz, 2H), 3.17 (d, J=21.9 Hz, 3H), 3.00-2.95 (m, 1H), 2.70 (t, J=14.0 Hz, 8H), 2.34-2.24 (m, 1H), 2.05 (d, J=34.4 Hz, 3H), 1.92-1.82 (m, 2H), 1.69-1.62 (m, 5H), 1.57 (d, J=11.5 Hz, 3H), 1.45 (d, J=6.2 Hz, 3H), 1.33 (d, J=12.6 Hz, 1H), 1.05-0.93 (m, 10H), 0.80 (d, J=9.8 Hz, 3H), 0.64 (d, J=12.2 Hz, 2H).

### Example A643. Synthesis of (2R)-2-(((1-(4-(dimethylamino)-4-methylpent-2-ynoyl)piperidin-4-yl)oxy)methyl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide

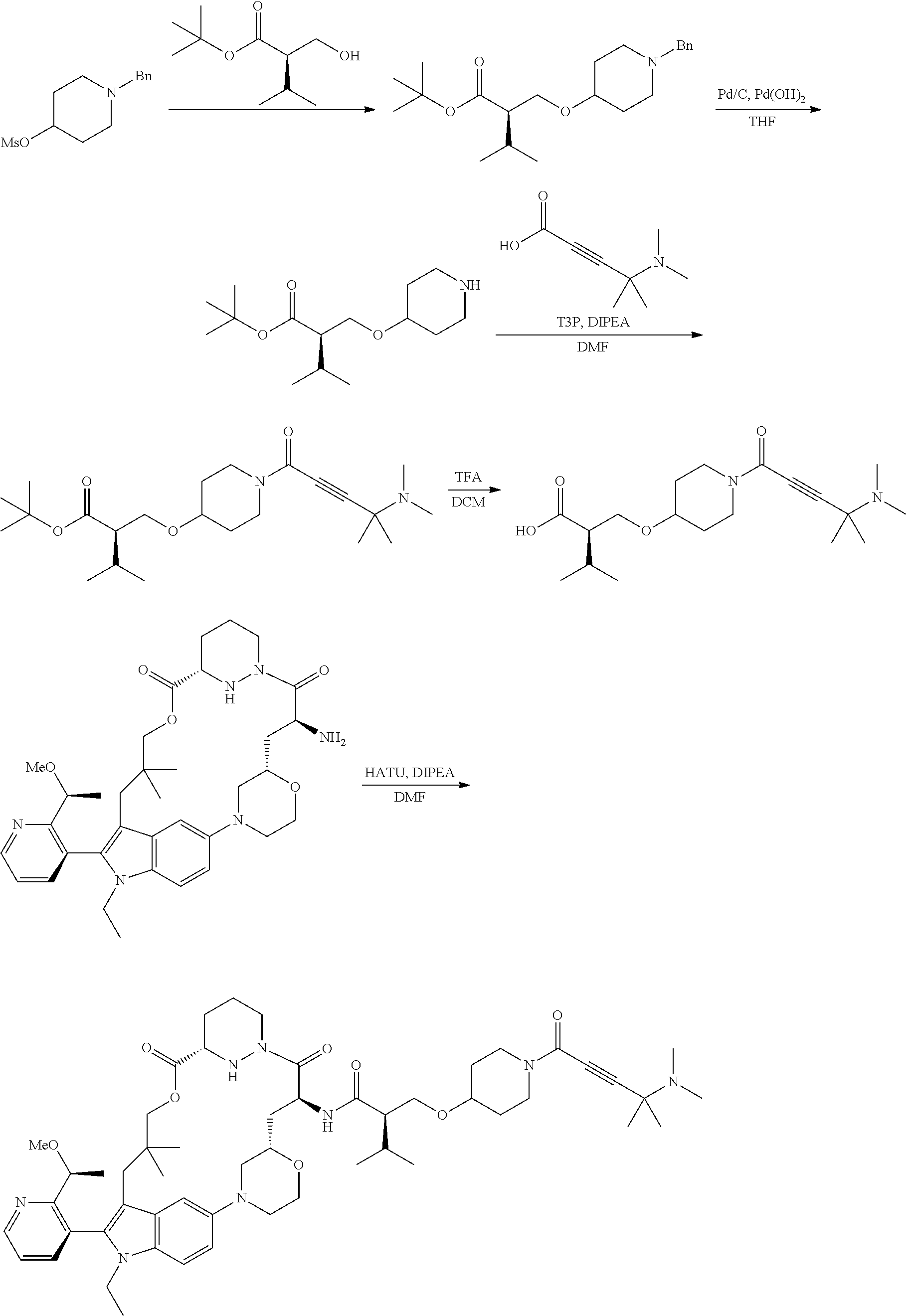

Step 1. A mixture of 1-(1-methylphenyl)piperidin-4-yl methanesulfonate (2 g, 7.4 mmol) and ter-butyl (2R)-2-(hydroxymethyl)-3-methylbutanoate (1.39 g, 7.4 mmol) was stirred at 120° C. for 1 h, then purified by silica gel column chromatography to give tert-butyl (R)-2-(((1-benzylpiperidin-4-yl)oxy)methyl)-3-methylbutanoate (800 mg, 28% yield) as an oil. LCMS (ESI): m/z: [M+H] calc'd for C₂₂H₃₅NO₃ 361.3; found 362.3.

Step 2. A mixture of tert-butyl (R)-2-(((1-benzylpiperidin-4-yl)oxy)methyl)-3-methylbutanoate (700 mg, 1.9 mmol), 10% wet Pd/C (411 mg, 3.9 mmol) and 20% wet Pd(OH)₂/C (542 mg, 3.9 mmol) in THF (30 mL) was stirred under an atmosphere of He (15 psi) for 16 h. The mixture was filtered and the filtrate was concentrated under reduced pressure to give tert-butyl (R)-3-methyl-2-((piperidin-4-yloxy)methyl)butanoate (440 mg, 80% yield) as a an oil. LCMS (ESI): m/z: [M+H] calc'd for C₁₅H₂₉NO₃ 271.2; found 272.2.

Step 3. To a mixture of tert-butyl (R)-3-methyl-2-((piperidin-4-yloxy)methyl)butanoate (440 mg, 1.6 mmol) 4-(dimethylamino)-4-methylpent-2-ynoic acid (3.77 g, 24.3 mmol) and DIPEA (2.09 g, 16.2 mmol) in DMF (50 mL) at 0° C. was added T3P (2.57 g, 8.1 mmol). The mixture was stirred at 0° C. for 1 h, then poured into H₂O (50 mL) and extracted with EtOAc (50 mL×3). The combined organic layers were washed with brine, concentrated under reduced pressure and the residue purified by silica gel column chromatography to give tert-butyl (R)-2-(((1-(4-(dimethylamino)-4-methylpent-2-ynoyl)piperidin-4-yl)oxy)methyl)-3-methylbutanoate (190 mg, 27% yield) as an oil. LCMS (ESI): m/z: [M+H] calc'd for C₂₃H₄₀N₂O₄ 408.3; found 409.4.

Step 4. To a mixture of tert-butyl (R)-2-(((1-(4-(dimethylamino)-4-methylpent-2-ynoyl)piperidin-4-yl)oxy)methyl)-3-methylbutanoate (180 mg, 0.47 mmol) in DCM (2 mL) was added TFA (1 mL). The mixture was stirred at room temperature for 1 h, then concentrated under reduced pressure to give (R)-2-(((1-(4-(dimethylamino)-4-methylpent-2-ynoyl)piperidin-4-yl)oxy)methyl)-3-methylbutanoic acid (170 mg, 98% yield) as an oil. LCMS (ESI): m/z: [M+H] calc'd for C₁₉H₃₂N₂O₄ 352.2; found 353.2.

Step 5. (2R)-2-(((1-(4-(dimethylamino)-4-methylpent-2-ynoyl)piperidin-4-yl)oxy)methyl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide was synthesized in a manner similar to (2R)-2-(((1-(4-(dimethylamino)-4-methylpent-2-ynoyl)azetidin-3-yl)oxy)methyl)-N-((2²S,6³S,4S)-12-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide except (2²S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione was substituted with (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione and 3-((1-[4-(dimethylamino)-4-methylpent-2-ynoyl]azetidin-3-yl)oxy)propanoic acid was substituted with (2R)-2-[({1-[4-(dimethylamino)-4-methylpent-2-ynoyl]piperidin-4-yl}oxy)methyl]-3-methylbutanoic acid. (101 mg, 42% yield) as a solid. LCMS (ESI): m/z: [M+H] calc'd for C₅₄H₇₈N₈O₈ 966.6; found 969.5; ¹H NMR (400 MHz, CD₃OD) δ 8.70 (d, J=4.8 Hz, 1H), 7.81-7.76 (m, 1H), 7.56-7.46 (m, 1H), 7.43-7.34 (m, 1H), 7.24-7.01 (m, 2H), 5.66-5.54 (m, 1H), 4.50-4.40 (m, 1H), 4.31-4.22 (m, 1H), 4.19-4.08 (m, 1H), 4.02-3.82 (m, 4H), 3.80-3.53 (m, 10H), 3.47-3.34 (m, 2H), 3.26-3.15 (m, 3H), 2.98-2.57 (m, 5H), 2.37-2.30 (m, 3H), 2.27-2.18 (m, 4H), 2.15-2.02 (m, 2H), 2.00-1.80 (m, 4H), 1.78-1.71 (m, 2H), 1.68-1.55 (m, 3H), 1.49-1.37 (m, 6H), 1.35-1.28 (m, 3H), 1.05-0.92 (m, 9H), 0.85-0.72 (m, 3H), 0.68-0.51 (m, 3H).

### Example A328. Synthesis of two atropisomers of (3S)-1-acryloyl-N-((2S)-1-(((6³S,4S)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide

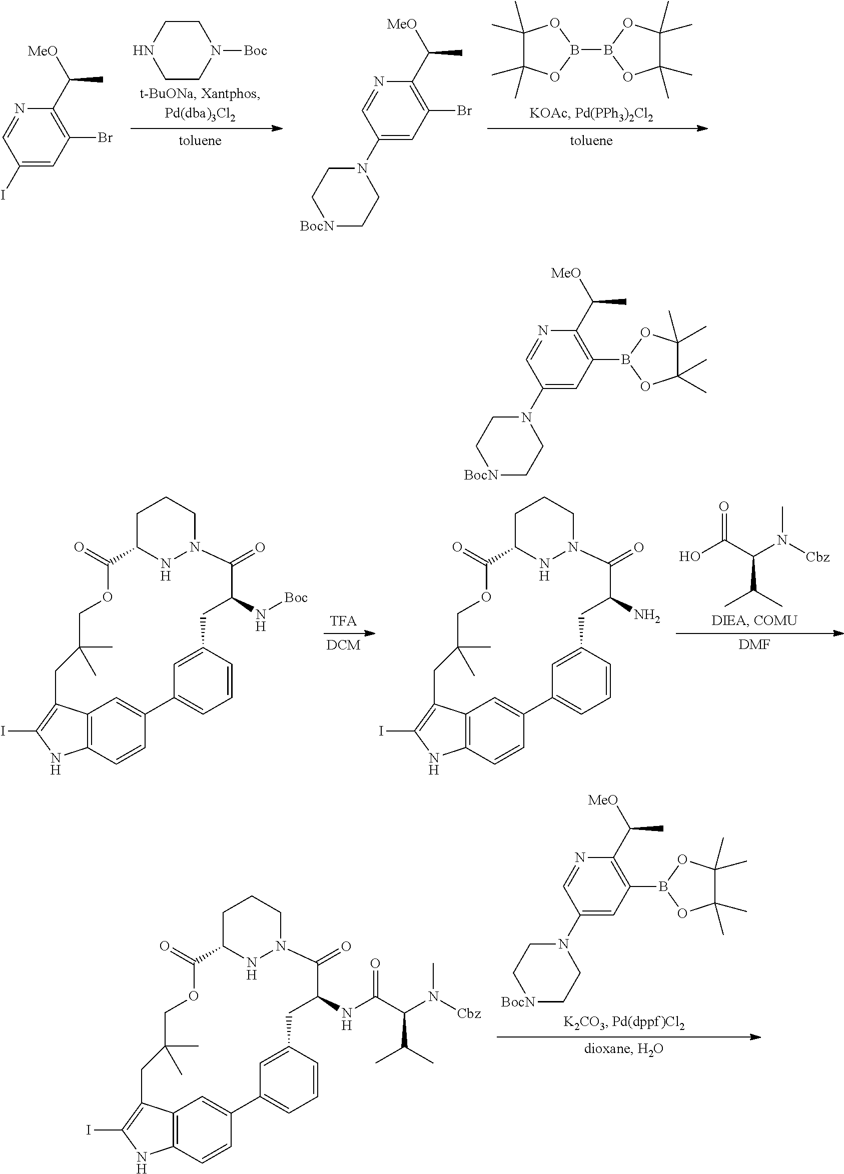

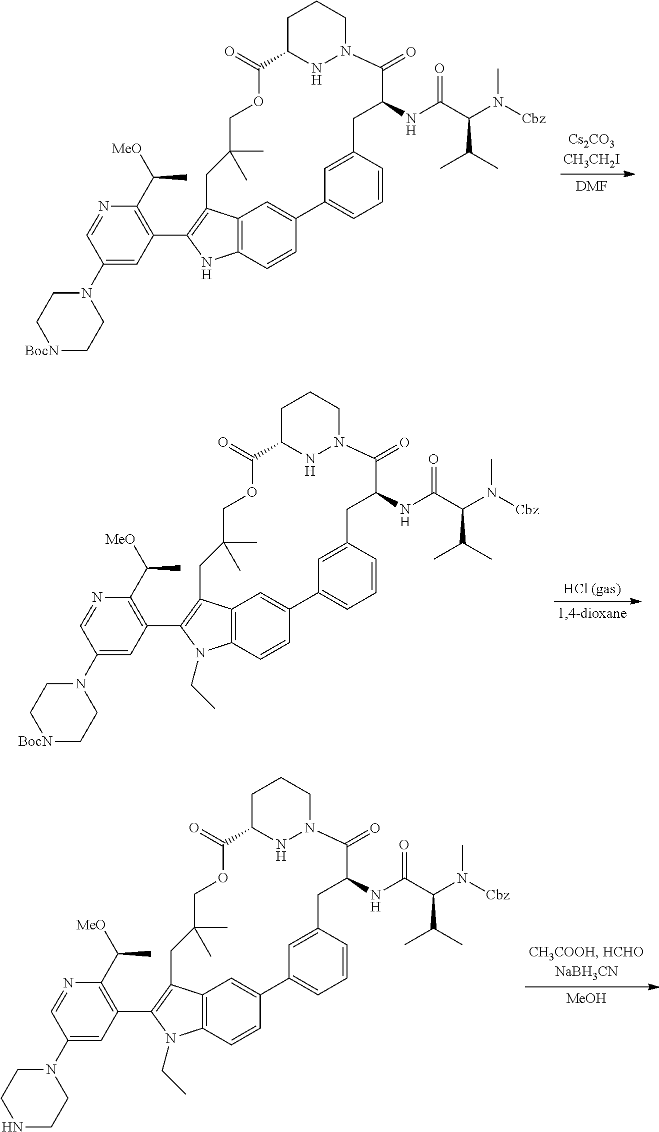

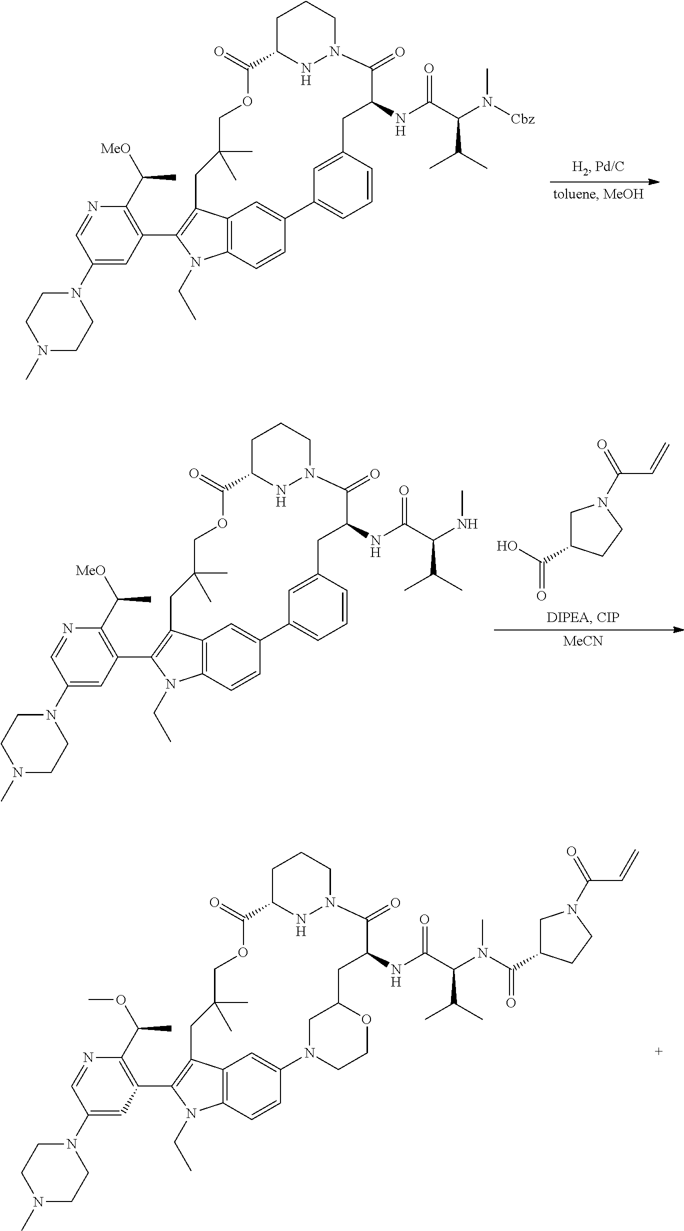

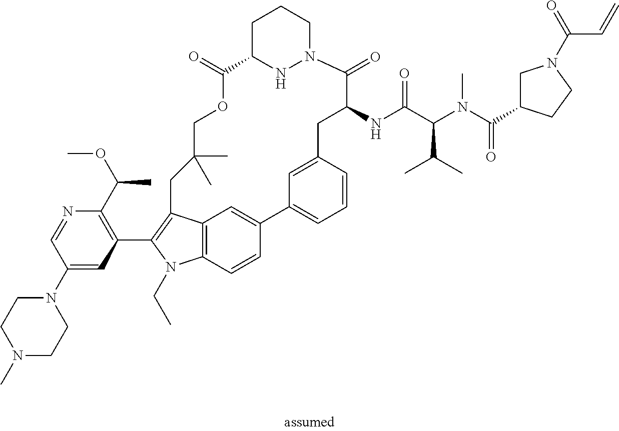

Step 1. To a mixture of 3-bromo-5-iodo-2-[(1S)-1-methoxyethyl]pyridine (2.20 g, 6.4 mmol) and tert-butyl piperazine-1-carboxylate (1.20 g, 6.4 mmol) in toluene (50 mL) under an atmosphere of Ar were added tBuONa (0.74 g, 7.7 mmol) and portion-wise addition of Pd₂(dba)₃ (0.59 g, 0.64 mmol), followed by portion-wise addition of Xantphos (0.74 g, 1.3 mmol). The mixture was heated to 100° C. and stirred for 16 h then H₂O added and the mixture extracted with EtOAc (400 mL×3). The combined organic layers were washed with brine (150 mL×3), dried over anhydrous Na₂SO₄ and filtered. The filtrate was concentrated under reduced pressure and the residue was purified by preparative-HPLC to give tert-butyl 4-[5-bromo-6-[(1S)-1-methoxyethyl]pyridin-3-yl]piperazine-1-carboxylate (1.7 g, 61% yield) as a solid. LCMS (ESI): m/z: [M+H] calc'd for C₁₇H₂₆BrN₃O₃ 399.1; found 400.1.

Step 2. A mixture of tert-butyl 4-[5-bromo-6-[(1S)-1-methoxyethyl]pyridin-3-yl]piperazine-1-carboxylate (1.76 g, 4.4 mmol) and 4,4,4′,4′,5,5,5′,5′-octamethyl-2,2′-bi(1,3,2-dioxaborolane) (1.67 g, 6.6 mmol) in toluene (18 mL) under an atmosphere of Ar were added KOAc (0.95 g, 9.7 mmol) and Pd(PPh₃)₂Cl₂ (0.31 g, 0.44 mmol) in portions. The mixture was heated to 80° C. and stirred for 16 h, then diluted with H₂O and the mixture extracted with EtOAc (500 mL×3). The combined organic layers were washed with brine (100 mL×3), dried over anhydrous Na₂SO₄ and filtered. The filtrate was concentrated under reduced pressure and the residue was purified by silica gel column chromatography to give tert-butyl 4-[6-[(1S)-1-methoxyethyl]-5-(4,4,5,5-tetramethyl-1,3,2-dioxaborolan-2-yl)pyridin-3-yl]piperazine-1-carboxylate (1.4 g, 68% yield) as a solid. LCMS (ESI): m/z: [M+H] calc'd for C₂₃H₃₈BN₃O₅ 447.4; found 448.2.

Step 3. To a mixture of tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.0 g, 1.5 mmol) in DCM (10 mL) at 0° C. under an atmosphere of N₂ was added TFA (5.0 mL, 67.3 mmol) in portions. The mixture was stirred at 0° C. for 1 h then concentrated under reduced pressure and dried azeotropically with toluene (3 mL×3) to give (6³S,4S)-4-amino-1²-iodo-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.0 g), which was used directly in the next step without further purification. LCMS (ESI): m/z: [M+H] calc'd for C₂₇H₃₁IN₄O₃ 586.1; found 587.3.

Step 4. To a mixture of (6³S,4S)-4-amino-1²-iodo-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.0 g, 1.7 mmol) in DMF (15 mL) at 0° C. under an atmosphere of N₂ were added DIPEA (2.20 g, 17.0 mmol) and (2S)-2-[[(benzyloxy)carbonyl](methyl)amino]-3-methylbutanoic acid (0.90 g, 3.4 mmol) in portions, followed by COMU (1.10 g, 2.6 mmol) in portions over 10 min. The mixture was stirred at 0° C. for 1.5 h, then diluted with H₂O and the mixture was extracted with EtOAc (300 mL×3). The combined organic layers were washed with brine (100 mL×3), dried over anhydrous Na₂SO₄ and filtered. The filtrate was concentrated under reduced pressure and the residue was purified by preparative-HPLC to give benzyl ((2S)-1-(((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)- pyridazina-2(1,3)-benzenacycloundecaphane-4-yl)amino)-3-methyl-1-oxobutan-2-yl)(methyl)carbamate (790 mg, 53% yield) as a solid.

Step 5. To a mixture of benzyl ((2S)-1-(((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (480 mg, 0.58 mmol) and tert-butyl 4-[6-[(1S)-1-methoxyethyl]-5-(4,4,5,5-tetramethyl-1,3,2-dioxaborolan-2-yl)pyridin-3-yl]piperazine-1-carboxylate (309 mg, 0.69 mmol) in 1,4-dioxane (8.0 mL) and H₂O (1.6 mL) under an atmosphere of Ar were added K₂CO₃ (199 mg, 1.4 mmol) and Pd(dppf)Cl₂ (42 mg, 0.06 mmol) in portions. The mixture was heated to 70° C. and stirred for 16 h, then diluted with H₂O and extracted with EtOAc (200 mL×3). The combined organic layers were washed with brine (150 mL×3), dried over anhydrous Na₂SO₄ and filtered. The filtrate was concentrated under reduced pressure and the residue was purified by silica gel column chromatography to give tert-butyl 4-(5-((6³S,4S)-4-((S)-2-(((benzyloxy)carbonyl)(methyl)amino)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-1²-yl)-6-((S)-1-methoxyethyl)pyridin-3-yl)piperazine-1-carboxylate (335 mg, 51% yield) as a solid. LCMS (ESI): m/z: [M+H] calc'd for C₅₈H₇₄N₈O₉ 1026.6; found 1027.4.

Step 6. To a mixture of tert-butyl 4-(5-((6³S,4S)-4-((S)-2-(((benzyloxy)carbonyl)(methyl)amino)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (335 mg, 0.33 mmol) in DMF (5 mL) at 0° C. under an atmosphere of N₂ were added Cs₂CO₃ (234 mg, 0.72 mmol) and iodoethane (102 mg, 0.65 mmol) in portions. The mixture was warmed to room temperature and stirred for 16 h, then diluted with H₂O and the mixture extracted with EtOAc (100 mL×3). The combined organic layers were washed with brine (50 mL×3), dried over anhydrous Na₂SO₄ and filtered. The filtrate was concentrated under reduced pressure and the residue was purified by preparative-TLC to give tert-butyl 4-(5-((6³S,4S)-4-((S)-2-(((benzyloxy)carbonyl)(methyl)amino)-3-methylbutanamido)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (320 mg, 84% yield) as a light yellow solid. LCMS (ESI): m/z: [M+H] calc'd for C₆₀H₇₈N₈O₉ 1054.6; found 1055.8.

Step 7. A mixture of tert-butyl 4-(5-((6³S,4S)-4-((S)-2-(((benzyloxy)carbonyl)(methyl)amino)-3-methylbutanamido)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (320 mg) in xx M HCl in 1,4-dioxane (3.0 mL) at 0° C. under an atmosphere of N₂ was stirred at room temperature for 2 h, then concentrated under reduced pressure to give benzyl ((2S)-1-(((6³S,4S)-1¹-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate, which was used directly in the next step further purification. LCMS (ESI): m/z: [M+H] calc'd for C₅₅H₇₀N₈O₇ 954.5; found 955.3.

Step 8. To a mixture of benzyl ((2S)-1-(((6³S,4S)-1¹-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (320 mg, 0.34 mmol) and HCHO (60 mg, 2.0 mmol) in MeOH (3.0 mL) at 0° C. under an atmosphere of N₂ were added NaCNBH₃ (42 mg, 0.67 mmol) and AcOH (60 mg, 1.0 mmol) in portions. The mixture was warmed to room temperature and stirred for 2 h, then diluted with H₂O and the mixture extracted with DCM/MeOH (5:1) (200 mL×3). The combined organic layers were washed with brine (100 mL×3), dried over anhydrous Na₂SO₄ and filtered. The filtrate was concentrated under reduced pressure and the residue was purified by preparative-HPLC to give benzyl ((2S)-1-(((6³S,4S)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (160 mg, 59% yield) as a solid. LCMS (ESI): m/z: [M+H] calc'd for C₅₆H₇₂N₈O₇ 968.6; found 969.6.

Step 9. To a mixture of benzyl ((2S)-1-(((6³S,4S)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (160 mg, 0.17 mmol) in toluene (10 mL) and MeOH (1.0 mL) was added Pd/C (130 mg, 1.2 mmol) in portions. The mixture was evacuated and re-filled with H₂ (×3), then stirred under an atmosphere of H₂ for 16 h. The mixture was filtered and the filtrate was concentrated under reduced pressure to give (2S)—N-((6³S,4S)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-(methylamino)butanamide (140 mg), which was used directly in the next step without further purification. LCMS (ESI): m/z: [M+H] calc'd for C₄₈H₆₆N₈O₅ 834.5; found 835.5.

Step 10. To a mixture of (2S)—N-((6³S,4S)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (140 mg, 0.17 mmol) in ACN (2.0 mL) at 0° C. under an atmosphere of N₂ were added DIPEA (433 mg, 3.35 mmol), (3S)-1-(prop-2-enoyl)pyrrolidine-3-carboxylic acid (57 mg, 0.34 mmol) in portions and CIP (70 mg, 0.25 mmol) in portions over 10 min. The mixture was stirred at 0° C. for 1.5 h, then H₂O added and the mixture extracted with EtOAc (150 mL×3). The combined organic layers were washed with brine (100 mL×3), dried over anhydrous Na₂SO₄ and filtered. The filtrate was concentrated under reduced pressure and the residue was purified by preparative-HPLC to give two atropisomers of (3S)-1-acryloyl-N-((2S)-1-(((6³S,4S)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (40 mg, 24% yield) as a solid and (20 mg, 12% yield) as a solid. LCMS (ESI): m/z: [M+H] calc'd for C₅₆H₇₅N₅O₉ 985.6; found 986.7; ¹H NMR (400 MHz, DMSO-d₆) 8.47 (t, J=2.1 Hz, 1H), 8.00 (d, J=4.7 Hz, 1H), 7.78-7.59 (m, 3H), 7.58-7.48 (m, 1H), 7.42-7.30 (m, 1H), 7.23 (dq, J=8.0, 4.0, 3.5 Hz, 1H), 7.15-7.03 (m, 1H), 6.75-6.50 (m, 1H), 6.18 (dt, J=16.8, 2.7 Hz, 1H), 5.70 (tt, J=9.3, 2.7 Hz, 1H), 5.48-5.23 (m, 1H), 5.06 (dd, J=31.1, 12.3 Hz, 1H), 4.74 (dd, J=11.0, 4.3 Hz, 1H), 4.33-4.15 (m, 2H), 4.01 (ddd, J=36.1, 12.6, 7.6 Hz, 2H), 3.91-3.56 (m, 6H), 3.52-3.39 (m, 2H), 3.31-3.28 (m, 2H), 3.24 (d, J=5.7 Hz, 4H), 3.06 (s, 4H), 2.93 (d, J=9.8 Hz, 2H), 2.81 (d, J=5.4 Hz, 3H), 2.47-2.43 (m, 4H), 2.22 (s, 4H), 2.09 (tq, J=12.0, 7.4, 6.6 Hz, 3H), 1.81 (s, 1H), 1.74 (d, J=11.7 Hz, 1H), 1.56 (d, J=11.7 Hz, 1H), 1.20 (dd, J=6.3, 1.5 Hz, 3H), 1.10 (td, J=7.2, 2.4 Hz, 3H), 1.00-0.86 (m, 6H), 0.86-0.72 (m, 3H), 0.54 (d, J=3.5 Hz, 3H) and LCMS (ESI): m/z: [M−H] calc'd for C₅₆H₇₅N₉O₇ 985.6; found 984.4; ¹H NMR (400 MHz, DMSO-d₆) δ 8.46 (d, J=3.0 Hz, 2H), 7.98 (s, 1H), 7.89-7.83 (m, 1H), 7.76-7.57 (m, 3H), 7.24 (s, 2H), 7.07 (s, 1H), 6.70-6.58 (m, 1H), 6.17 (d, J=16.5 Hz, 1H), 5.73-5.67 (m, 1H), 5.36-5.30 (m, 1H), 4.31-3.97 (m, 6H), 3.83-3.77 (m, 2H), 3.74-3.49 (m, 6H), 3.48-3.41 (m, 1H), 3.40-3.37 (m, 2H) 3.28-3.24 (m, 4H), 3.07 (s, 3H), 2.88-2.82 (m, 1H), 2.80-2.64 (m, 7H), 2.49-2.44 (m, 4H), 2.22 (s, 3H), 2.04 (d, J=26.1 Hz, 3H), 1.85-1.79 (m, 1H), 1.67-1.55 (m, 2H), 1.35 (d, J=6.1 Hz, 3H), 1.27-1.22 (m, 1H), 1.05-0.93 (m, 4H), 0.89 (d, J=6.9 Hz, 2H), 0.79 (d, J=12.4 Hz, 5H), 0.73 (d, J=6.5 Hz, 1H), 0.56 (s, 3H).

### Example A542. The synthesis of 1-(4-(dimethylamino)-4-methylpent-2-ynoyl)-4-fluoro-N-((2S)-1-(((6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6a-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperidine-4-carboxamide

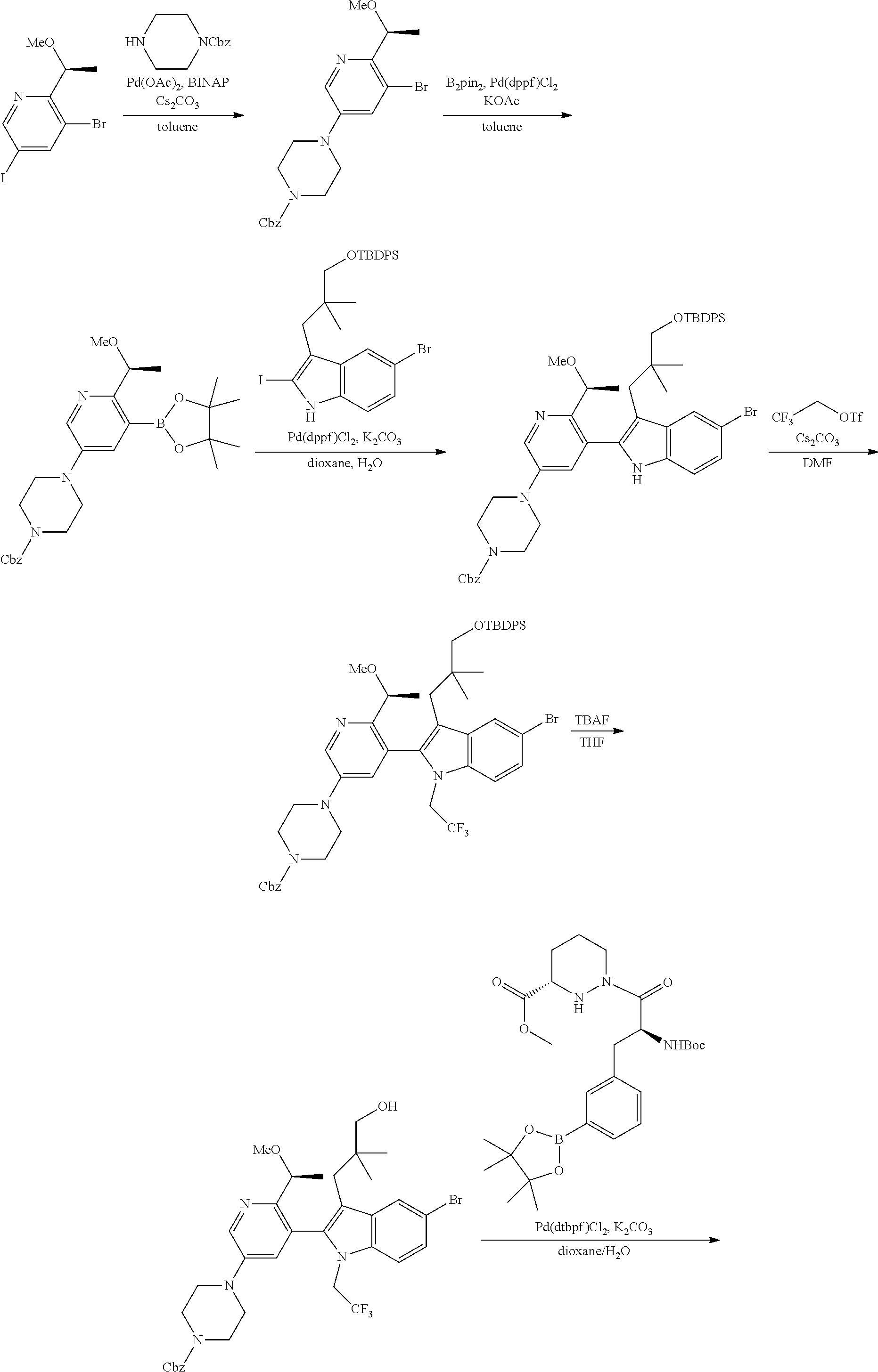

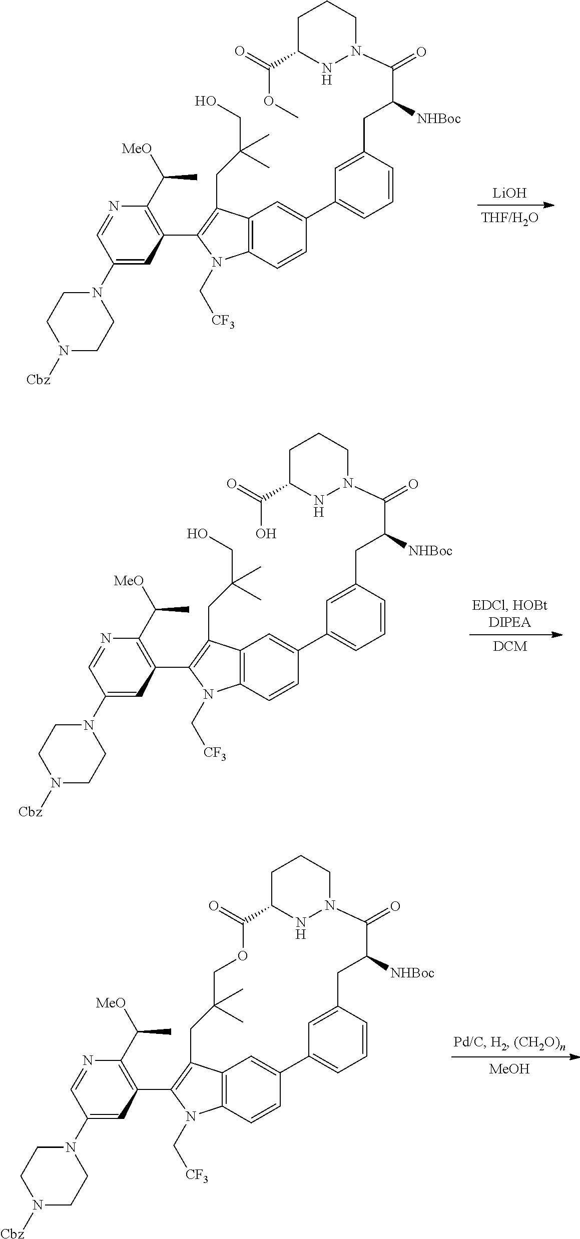

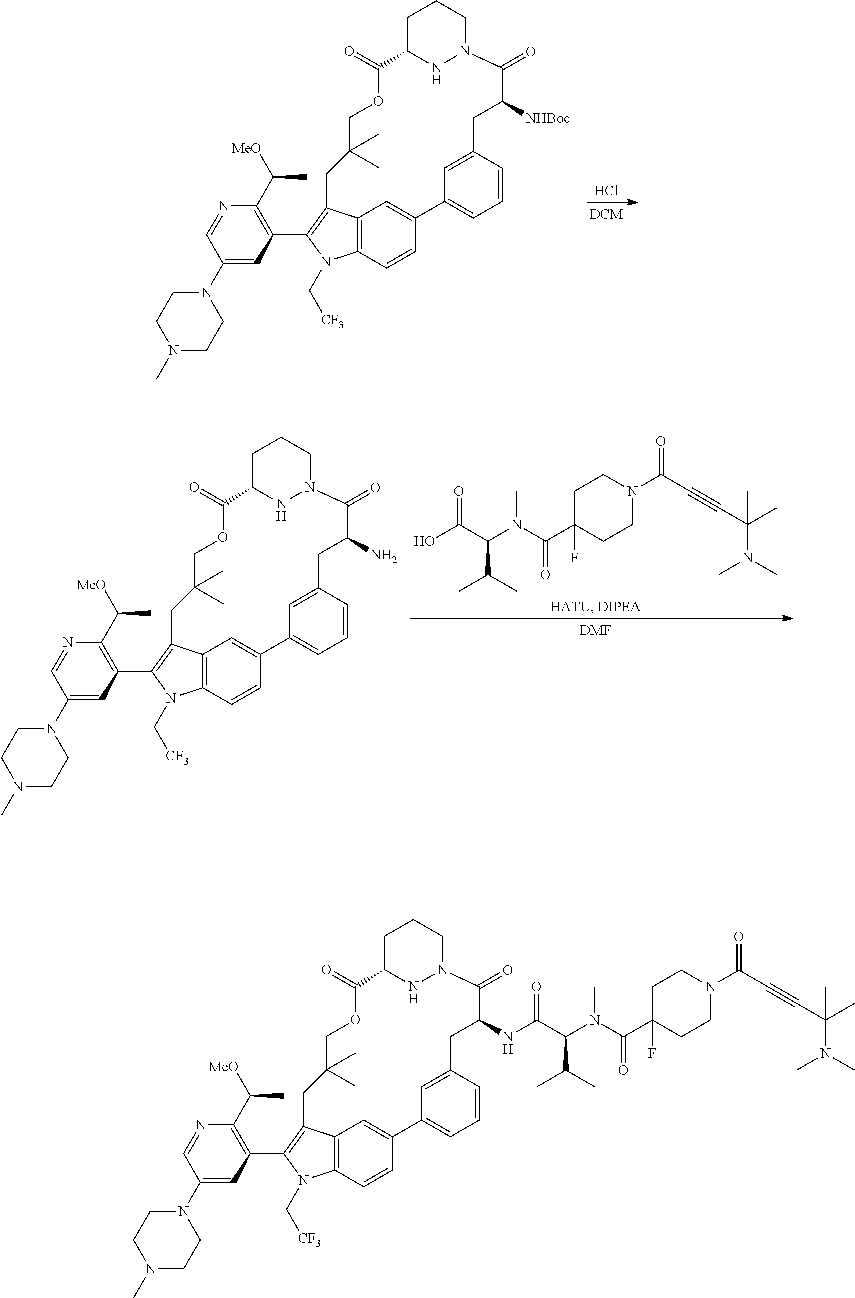

Step 1. To a solution of (S)-3-bromo-5-iodo-2-(1-methoxyethyl)pyridine (15 g, 43.86 mmol), and benzyl piperazine-1-carboxylate (8.7 g, 39.48 mmol) in toluene (150 mL) at 0° C., were added cesium carbonate (71.46 g, 219.32 mmol), BINAP (0.55 g, 0.88 mmol) and palladium acetate (0.49 g, 2.19 mmol) in portions. The reaction mixture was stirred at 90° C. for 12 h under an argon atmosphere. The resulting mixture was cooled down to room temperature, filtered and the filter cake was washed with EtOAc (150 mL×3). The filtrate was concentrated under reduced pressure. The residue was purified by silica gel column chromatography to afford benzyl (S)-4-(5-bromo-6-(1-methoxyethyl)pyridin-3-yl)piperazine-1-carboxylate (16 g, 84% yield) as solid. LCMS (ESI): m/z: [M+H] calc'd for C₄₄H₅₈N₆O₇ 433.1; found 434.0.

Step 2. To a stirred solution of benzyl (S)-4-(5-bromo-6-(1-methoxyethyl)pyridin-3-yl)piperazine-1-carboxylate (22.7 g, 52.26 mmol), bis(pinacolato)diboron (19.91 g, 78.4 mmol) in toluene (230 mL) at 0° C., were added potassium acetate (12.82 g, 130.66 mmol) and Pd(dppf)Cl₂·DCM (4.26 g, 5.23 mmol) in portions. The reaction mixture was stirred at 90° C. for 6 h under an argon atmosphere. The resulting mixture was filtered and the filter cake was washed with EtOAc (200 mL×3). The filtrate was concentrated under reduced pressure. The residue was purified by silica gel column chromatography to afford benzyl (S)-4-(6-(1-methoxyethyl)-5-(4,4,5,5-tetramethyl-1,3,2-dioxaborolan-2-yl)pyridin-3-yl)piperazine-1-carboxylate (14.7 g, 58% yield) as solid. LCMS (ESI): m/z: [M+H] calc'd for C₂₆H₃₆BN₃O₅ 481.3; found 482.3.

Step 3. To a stirred solution of 5-bromo-3-(3-((tert-butyldiphenylsilyl)oxy)-2,2-dimethylpropyl)-2-iodo-1H-indole (17.46 g, 27 mmol) in 1,4-dioxane (150 mL) and H₂O (30 mL) at 0° C., were added potassium carbonate (9.33 g, 67.51 mmol) and Pd(dppf)Cl₂ DCM (2.2 g, 2.7 mmol) in portions, followed by benzyl (S)-4-(6-(1-methoxyethyl)-5-(4, 4, 5, 5-tetramethyl-1, 3, 2-dioxaborolan-2-yl) pyridin-3-yl) piperazine-1-carboxylate (13 g, 27 mmol). The reaction mixture was stirred at 70° C. for 12 h under an argon atmosphere. The resulting mixture was cooled to room temperature and quenched with H₂O, then extracted with EtOAc (200 mL×3). The organic phase was dried over anhydrous sodium sulfate and concentrated under reduced pressure. The residue was purified by silica gel column chromatography to afford benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (20 g, 84.7% yield) as solid. LCMS (ESI): m/z: [M+H] calc'd for C₄₉H₅₇BN₄O₄Si 873.2; found 873.3.

Step 4. To a mixture of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (19 g, 21.74 mmol) and Cs₂CO₃ (49.58 g, 152.17 mmol) in DMF (190 mL) at 0° C. under argon atmosphere, was dropwise added 2,2,2-trifluoroethyl trifluoromethanesulfonate (50.46 g, 217.39 mmol). The reaction mixture was stirred at room temperature for 12 h under an argon atmosphere, then quenched with H₂O, extracted with EtOAc (200 mL×3). The combined organic layers were washed with brine (200 mL×3), dried over anhydrous sodium sulfate and concentrated under reduced pressure. The residue was purified by silica gel column chromatography to afford benzyl (S)-4-(5-(5-bromo-3-(3-((tert-butyldiphenylsilyl)oxy)-2,2-dimethylpropyl)-1-(2,2,2-trifluoroethyl)-1H-indol-2-yl)-6-(1-methoxyethyl)pyridin-3-yl)piperazine-1-carboxylate (17.6 g, 84.7% yield) as solid. LCMS (ESI): m/z: [M+H] calc'd for C₅₁H₅₈BF₃N₄O₄Si 954.2; found 955.3.

Step 5. To a solution of benzyl (S)-4-(5-(5-bromo-3-(3-((tert-butyldiphenylsilyl)oxy)-2,2-dimethylpropyl)-1-(2,2,2-trifluoroethyl)-1H-indol-2-yl)-6-(1-methoxyethyl)pyridin-3-yl)piperazine-1-carboxylate (18 g, 18.83 mmol), was added TBAF in THF (180.0 mL) at 0° C. The reaction mixture was stirred at 40° C. for 12 h under an argon atmosphere, then quenched with cold H₂O. The resulting mixture was extracted with EtOAc (200 mL×3). The combined organic layers were washed with brine (200 mL×3), dried over anhydrous sodium sulfate and concentrated under reduced pressure. The residue was purified by silica gel column chromatography to afford benzyl (S)-4-(5-(5-bromo-3-(3-hydroxy-2,2-dimethylpropyl)-1-(2,2,2-trifluoroethyl)-1H-indol-2-yl)-6-(1-methoxyethyl)pyridin-3-yl)piperazine-1-carboxylate (7.8 g, 57.7% yield) as solid. LCMS (ESI): m/z: [M+H] calc'd for C₃₅H₄₀BrF₃N₄O₄ 716.2; found 717.1.

Step 6. A solution of benzyl (S)-4-(5-(5-bromo-3-(3-hydroxy-2,2-dimethylpropyl)-1-(2,2,2-trifluoroethyl)-1H-indol-2-yl)-6-(1-methoxyethyl)pyridin-3-yl)piperazine-1-carboxylate (1 g, 1.39 mmol) in 1,4-dioxane (10 mL) and H₂O (2 mL), was added methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (1.08 g, 2.09 mmol), potassium carbonate (481.47 mg, 3.48 mmol) and Pd(dtbpf)Cl₂ (181.64 mg, 0.28 mmol) in portions at 0° C. The reaction mixture was stirred at 70° C. for 3 h under an argon atmosphere. The resulting mixture was cooled to room temperature, then quenched with H₂O and extracted with EtOAc (50 mL×3). The combined organic layers were washed with brine (20 mL×3), dried over anhydrous sodium sulfate and concentrated under reduced pressure. The residue was purified by silica gel chromatography to afford methyl (S)-1-((S)-3-(3-(2-(5-(4-((benzyloxy)carbonyl)piperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)-2-((tert-butoxycarbonyl)amino)propanoyl)hexahydropyridazine-3-carboxylate (1.1 g, 77% yield) as solid. LCMS (ESI): m/z: [M+H] calc'd for C₅₅H₆₈F₃N₇O₉ 1027.5; found 1028.3.

Step 7. To a solution of methyl (S)-1-((S)-3-(3-(2-(5-(4-((benzyloxy)carbonyl)piperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)-2-((tert-butoxycarbonyl)amino)propanoyl)hexahydropyridazine-3-carboxylate (1.1 g, 1.07 mmol) in THF (8 mL) and H₂O (2 mL) at 0° C., was dropwise added LiOH (2.2 mL, 1M aqueous) under an argon atmosphere. The reaction mixture was stirred for 2 h then concentrated under reduced pressure. The residue was acidified to pH 5 with citric acid (1M) and extracted with EtOAc (20 mL×3). The combined organic layers were concentrated under reduced pressure. The residue was purified by reverse phase chromatography to afford (S)-1-((S)-3-(3-(2-(5-(4-((benzyloxy)carbonyl)piperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)-2-((tert-butoxycarbonyl)amino)propanoyl)hexahydropyridazine-3-carboxylic acid (750 mg, 69% yield) as solid. LCMS (ESI): m/z: [M+H] calc'd for C₅₄H₆₆F₃N₇O₉ 1013.5; found 1014.3.

Step 8. To a solution of (S)-1-((S)-3-(3-(2-(5-(4-((benzyloxy)carbonyl)piperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)-2-((tert-butoxycarbonyl)amino)propanoyl)hexahydropyridazine-3-carboxylic acid (0.75 g, 0.74 mmol) in DCM (75 mL) at 0° C., were added in portions HOBT (0.5 g, 3.7 mmol), DIPEA (3.82 g, 29.58 mmol), and EDCI (4.25 g, 22.19 mmol) at 0° C. The reaction mixture was stirred at room temperature for 12 h under an argon atmosphere. The resulting mixture was concentrated under reduced pressure and extracted with EtOAc (100 mL×3). The combined organic layers were washed with brine (50 mL×3), dried over anhydrous sodium sulfate and concentrated under reduced pressure. The residue was purified by silica gel chromatography to afford benzyl 4-(5-((6³S,4S)-4-((tert-butoxycarbonyl)amino)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)- benzenacycloundecaphane-1²-yl)-6-((S)-1-methoxyethyl)pyridin-3-yl)piperazine-1-carboxylate (0.5 g, 67.9% yield) as solid. LCMS (ESI): m/z: [M+H] calc'd for C₅₄H₆₄F₃N₇O₈ 995.5; found 996.3.

Step 9. To a mixture of benzyl 4-(5-((6³S,4S)-4-((tert-butoxycarbonyl)amino)-10,10-dimethyl-5,7-dioxo-1′-(2,2,2-trifluoroethyl)-6¹,6²,6³,6⁴,6⁵,6⁶-hexahydro-1¹H-8-oxa-1(5,3)-indola-6(1,3)-pyridazina-2(1,3)- benzenacycloundecaphane-1²-yl)-6-((S)-1-methoxyethyl)pyridin-3-yl)piperazine-1-carboxylate (500 mg, 0.5 mmol) in MeOH (15 mL) at 0° C., was added paraformaldehyde (135.64 mg, 1.5 mmol), and Pd/C (750 mg) in portions. The reaction mixture was stirred at room temperature for 12 h under a hydrogen atmosphere. The resulting mixture was filtered and the filter cake was washed with EtOAc (50 mL×5). The filtrate was concentrated under reduced pressure. The residue was purified by silica gel chromatography to afford tert-butyl ((6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 79.6% yield) as an solid. LCMS (ESI): m/z: [M+H] calc'd for C₄₇H₆₀F₃N₇O₈ 875.5; found 876.5.

Step 10. To a solution of tert-butyl ((6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (300 mg, 0.34 mmol) in DCM (2 mL) at 0° C., was dropwise added HCl in 1,4-dioxane (1 mL, 4M, 4 mmol). The reaction mixture was stirred at room temperature for 2 h, then concentrated under reduced pressure to give (6³S,4S)-4-amino-1²-(2-((S)-1-methoxyethyl)-5-(4-methylpiperazin-1-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione hydrochloride (350 mg, crude) as solid. LCMS (ESI): m/z: [M+H] calc'd for C₄₂H₅₂F₃N₇O₄ 775.4; found 766.4.

Step 11. To a solution of (6³S,4S)-4-amino-1²-(2-((S)-1-methoxyethyl)-5-(4-methylpiperazin-1-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione hydrochloride (150 mg, 0.19 mmol) and N-(1-(4-(dimethylamino)-4-methylpent-2-ynoyl)-4-fluoropiperidine-4-carbonyl)-N-methyl-L-valine (154 mg, 0.39 mmol) in DMF (2 mL) at 0° C., was dropwise added a mixture of DIPEA (1 g, 7.72 mmol) and HATU (110 mg, 0.29 mmol) in DMF (0.2 mL). The reaction mixture was stirred at 0° C. for 2 h under an argon atmosphere, then quenched with H₂O. The resulting mixture was extracted with EtOAc (20 mL×3). The combined organic phase was washed with brine (10 mL×3), dried over anhydrous sodium sulfate and concentrated under reduced pressure. The residue was purified by reverse phase chromatography to afford 1-(4-(dimethylamino)-4-methylpent-2-ynoyl)-4-fluoro-N-((2S)-1-(((6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperidine-4-carboxamide (39.5 mg, 17% yield) as solid. ¹H NMR (400 MHz, DMSO-d₆) δ 8.48 (d, J=2.9 Hz, 1H), 8.32 (t, J=7.3 Hz, 1H), 7.97 (s, 1H), 7.82-7.69 (m, 3H), 7.66 (t, J=7.8 Hz, 1H), 7.33-7.07 (m, 3H), 5.50 (dd, J=16.7, 8.6 Hz, 1H), 5.33 (t, J=9.2 Hz, 1H), 5.16 (d, J=12.2 Hz, 1H), 4.94-4.80 (m, 1H), 4.64 (d, J=10.8 Hz, 1H), 4.33-4.16 (m, 3H), 4.12-4.02 (m, 2H), 3.71-3.50 (m, 3H), 3.25 (s, 3H), 3.21-3.16 (m, 3H), 3.14-3.05 (m, 1H), 2.96 (t, J=4.7 Hz, 4H), 2.84 (s, 1H), 2.82-2.72 (m, 2H), 2.59-2.53 (m, 1H), 2.47-2.40 (m, 4H), 2.22 (d, J=2.9 Hz, 9H), 2.18-2.12 (m, 2H), 2.11-1.99 (m, 3H), 1.87-1.78 (m, 1H), 1.74-1.62 (m, 1H), 1.59-1.48 (m, 1H), 1.40-1.32 (m, 9H), 1.01 (t, J=7.7 Hz, 1H), 0.89 (s, 5H), 0.83 (d, J=6.3 Hz, 1H), 0.77 (d, J=6.6 Hz, 2H), 0.38 (s, 3H). LCMS (ESI): m/z: [M+H] calc'd for C₄₄H₅₈N₆O₇ 1154.6; found 1155.7.

### Example A735. Synthesis of 2-acryloyl-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methyl-5-oxa-2,9-diazaspiro[3.5]nonane-9-carboxamide

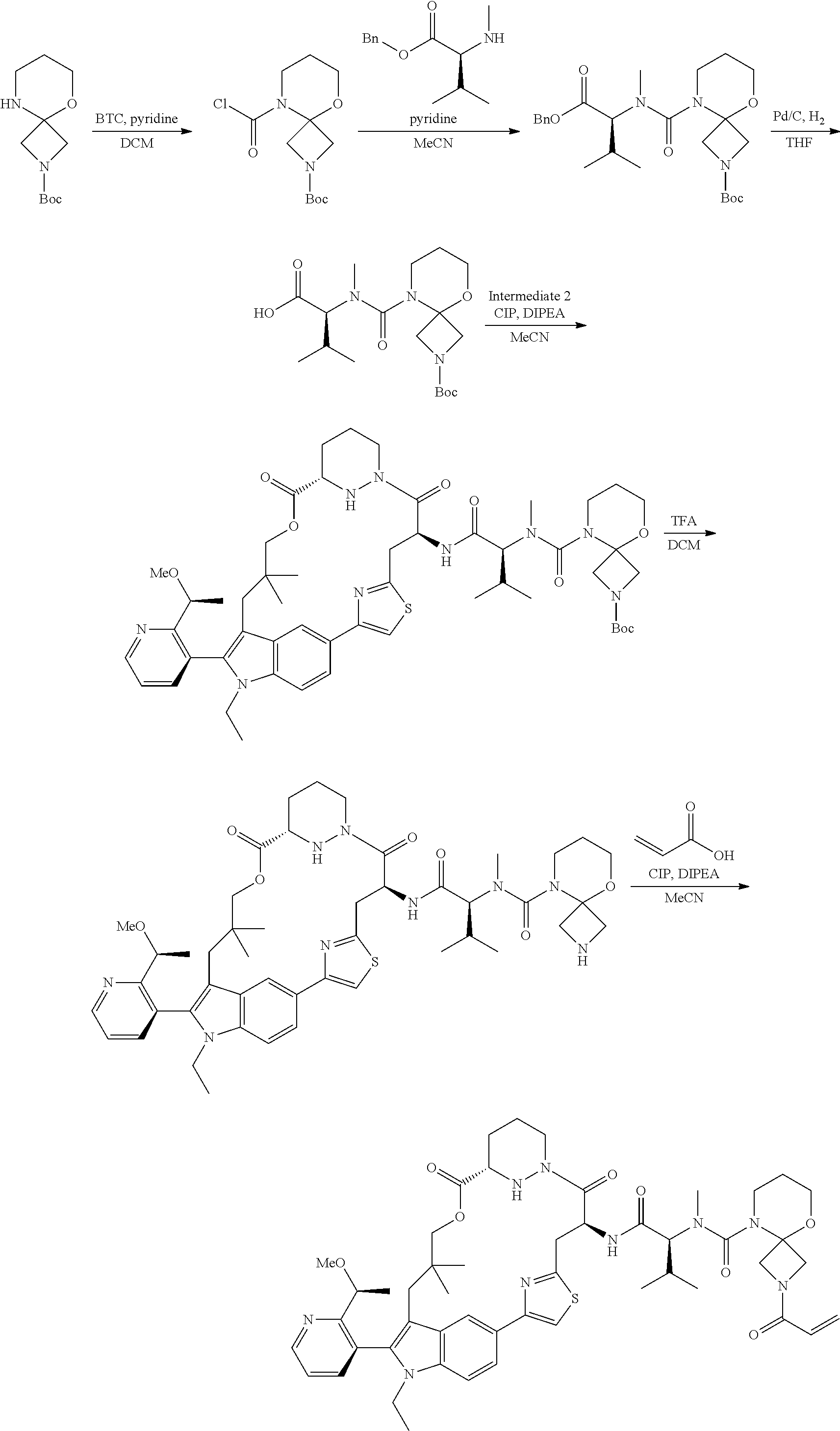

Step 1. To a stirred mixture of BTC (425.2 mg, 1.448 mmol) in DCM (10 mL) was added dropwise pyridine (1.04 g, 13.16 mmol) and tert-butyl 5-oxa-2,9-diazaspiro[3.5]nonane-2-carboxylate (1 g, 4.39 mmol), the reaction mixture was stirred at room temperature for 2 h. The resulting mixture was concentrated under reduced pressure to give crude tert-butyl 9-(chlorocarbonyl)-5-oxa-2,9-diazaspiro[3.5]nonane-2-carboxylate.

Step 2. To a stirred solution of tert-butyl 9-(chlorocarbonyl)-5-oxa-2,9-diazaspiro[3.5]nonane-2-carboxylate (2.5 g, crude) in MeCN (20 mL) were added dropwise pyridine (1.04 g, 13.16 mmol) and benzyl (2S)-3-methyl-2-(methylamino)butanoate (970.66 mg, 4.38 mmol) at room temperature. The reaction mixture was stirred at 80° C. for 12 h and concentrated under reduced pressure. The residue was purified by silica gel column chromatography to afford tert-butyl (S)-9-((1-(benzyloxy)-3-methyl-1-oxobutan-2-yl)(methyl)carbamoyl)-5-oxa-2,9-diazaspiro[3.5]nonane-2-carboxylate (783 mg, 37.6% yield, two steps) as an oil. LCMS (ESI): m/z: [M+H] calc'd for C₂₅H₃₇N₃O₆ 475.3; found 476.3.

Step 3. A solution of tert-butyl (S)-9-((1-(benzyloxy)-3-methyl-1-oxobutan-2-yl)(methyl)carbamoyl)-5-oxa-2,9-diazaspiro[3.5]nonane-2-carboxylate (783 mg, 1.65 mmol) and 10 wt % palladium on carbon (226.29 mg) in THF (10 mL) was stirred for 2 h at 50° C. under a hydrogen atmosphere. The resulting mixture was cooled to room temperature, filtered and the filter cake was washed with MeCN (10 mL×3). The filtrate was concentrated under reduced pressure to give N-(2-(tert-butoxycarbonyl)-5-oxa-2,9-diazaspiro[3.5]nonane-9-carbonyl)-N-methyl-L-valine (591 mg, 98.8% yield) as solid. LCMS (ESI): m/z: [M+H] calc'd for C₁₈H₃₁N₃O₆ 385.2; found 386.3.

Step 4. To a stirred solution of intermediate 2 (731 mg, 1.16 mmol) and DIPEA (2.25 g, 17.38 mmol) in MeCN (50 mL) was added CIP (644.31 mg, 2.32 mmol) and N-(2-(tert-butoxycarbonyl)-5-oxa-2,9-diazaspiro[3.5]nonane-9-carbonyl)-N-methyl-L-valine (446.68 mg, 1.16 mmol) at room temperature. The reaction mixture was stirred for 2 h then concentrated under reduced pressure. The residue was purified by silica gel column chromatography to afford tert-butyl 9-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)-5-oxa-2,9-diazaspiro[3.5]nonane-2-carboxylate (752 mg, 65% yield) as solid. LCMS (ESI): m/z: [M+H] calc'd for C₅₂H₇₁N₉O₉S 997.5; found 996.6.

Step 5. To a stirred solution of tert-butyl 9-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)-5-oxa-2,9-diazaspiro[3.5]nonane-2-carboxylate (752 mg, 0.75 mmol) in DCM (40 mL) was added TFA (10 mL) in portions at room temperature. The reaction mixture was stirred for 2 h, then concentrated under reduced pressure. To the residue was added saturated aqueous sodium bicarbonate (100 mL) and DCM (100 mL). The aqueous layer was separated and extracted with DCM (100 mL×2). The combined organic phase was dried over anhydrous sodium sulfate, filtrated and concentrated under reduced pressure to afford N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methyl-5-oxa-2,9-diazaspiro[3.5]nonane-9-carboxamide (587 mg, 87% yield) as solid. LCMS (ESI): m/z [M+H] calc'd for C₄₇H₆₃N₉O₇S 897.5; found 898.4.

Step 6. A stirred solution of N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N-methyl-5-oxa-2,9-diazaspiro[3.5]nonane-9-carboxamide (586 mg, 0.65 mmol) in MeCN (10 mL) was added acrylic acid (47 mg, 0.65 mmol), DIPEA (421 mg, 3.26 mmol), CIP (362 mg, 1.3 mmol). The reaction mixture was stirred for 12 h and concentrated under reduced pressure. The residue was purified by reverse phase chromatography to afford 2-acryloyl-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N-methyl-5-oxa-2,9-diazaspiro[3.5]nonane-9-carboxamide (194 mg, 27% yield) as a solid. ¹H NMR (400 MHz, DMSO-d₆) δ 8.78 (dd, J=4.8, 1.7 Hz, 1H), 8.48 (d, J=1.6 Hz, 2H), 7.85-7.65 (m, 3H), 7.65-7.42 (m, 2H), 6.30 (mm, 1H), 6.10 (m, J=17.0, 1H), 5.78-5.50 (m, J=10.3, 2H), 5.10 (dd, 1H), 4.40-3.80 (m, 14H), 3.60-3.10 (m, 10H), 2.94 (d, J=14.5 Hz, 1H), 2.85 (s, 4H), 2.42 (dd, 1H), 2.07 (dd, 2H), 1.80 (s, 2H), 1.55 (s, 3H), 1.32 (d, 3H), 0.95-0.75 (m, 12H), 0.33 (s, 3H). LCMS (ESI): m/z: [M+H] calc'd for C₅₀H₆₅N₉O₈S 951.5; found 952.6.

### Example A720. Synthesis of (2R)-2-(((1-(4-(dimethylamino)-4-methylpent-2-ynoyl)azetidin-3-yl)oxy)methyl)-N-((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹,2¹H-8-oxa-1(5,3)-indola-6(1,3)-pyridazina-2(5,3)-triazolacycloundecaphane-4-yl)-3-methylbutanamide

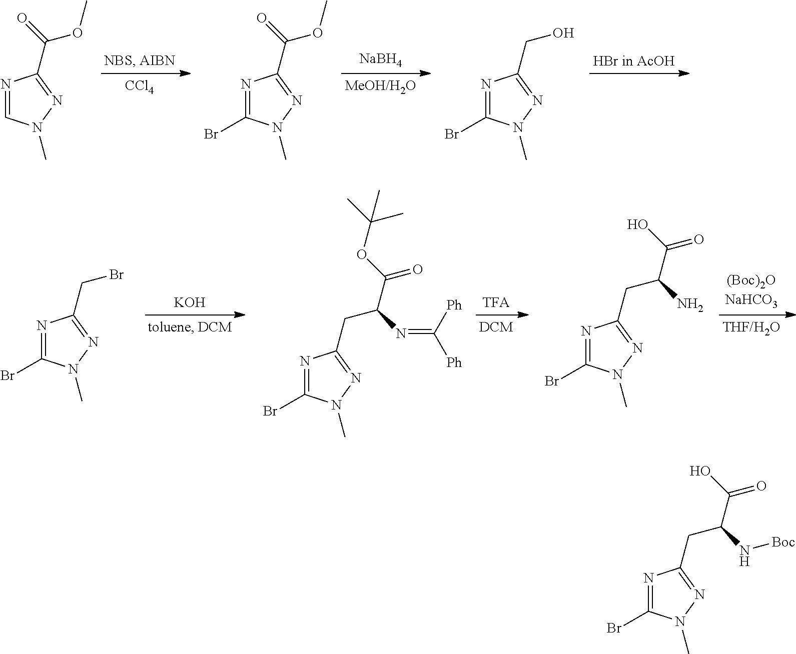

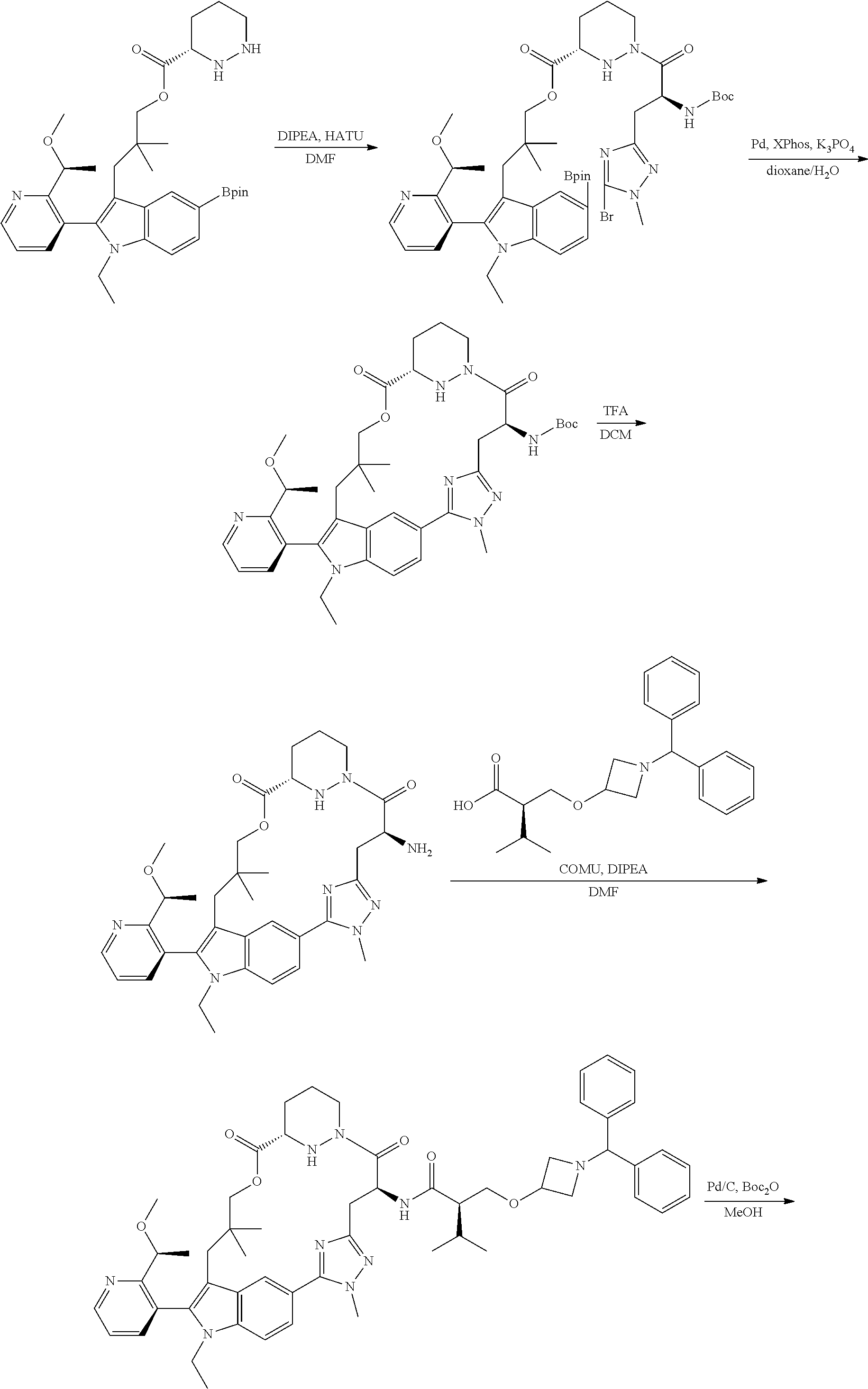

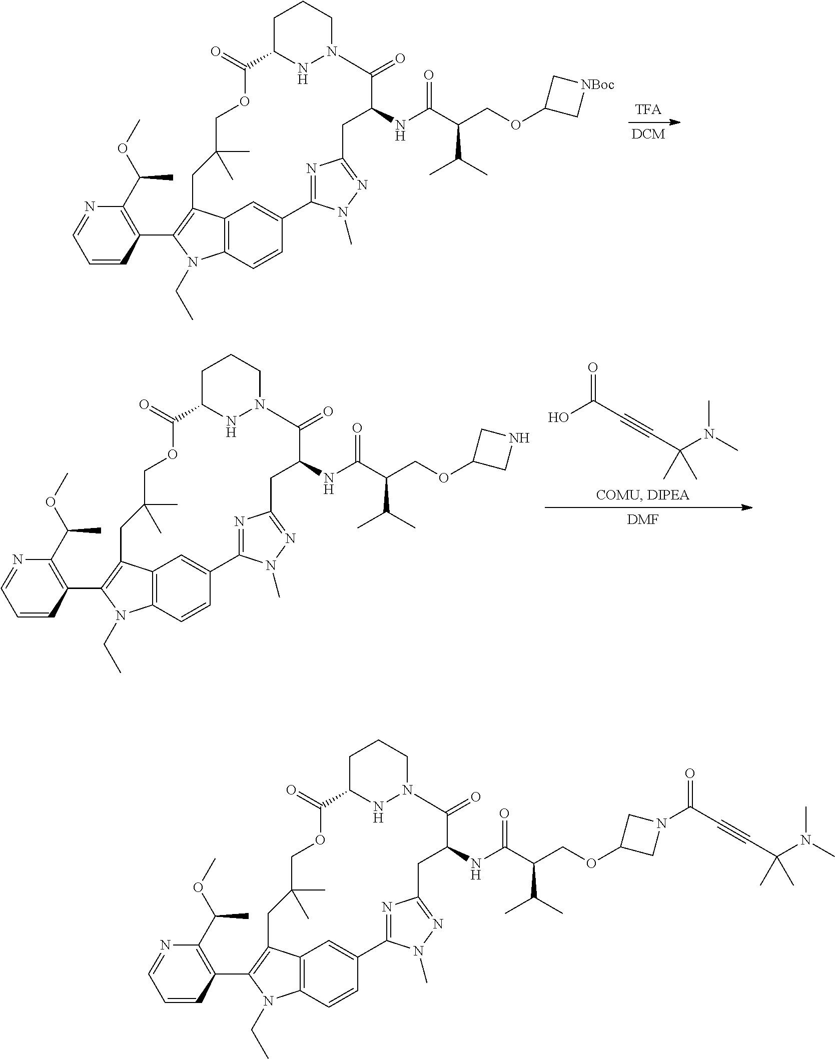

Step 1. To a stirred solution of methyl 1-methyl-1,2,4-triazole-3-carboxylate (7.0 g, 49.60 mmol) in CCl₄ (70. mL) was added NBS (13.24 g, 74.40 mmol) and AIBN (11.40 g, 69.44 mmol) in portions at 25° C. under an argon atmosphere. The resulting mixture was stirred for 24 h at 80° C. The resulting mixture was filtered, the filtrate was cooled to 20° C. and kept at 20° C. for 30 min. The resulting mixture was filtered. The filter cake was washed with H₂O (3×50 mL) and pet. ether (3×100 mL). The filter cake was dried under reduced pressure. This resulted in methyl 5-bromo-1-methyl-1,2,4-triazole-3-carboxylate (10 g, crude) as a light yellow solid. LCMS (ESI): m/z: [M+H] calc'd for C₅H₆BrN₃O₂ 219.0; found 219.9.

Step 2. To a stirred solution of methyl 5-bromo-1-methyl-1,2,4-triazole-3-carboxylate (10.0 g, 45.50 mmol) in MeOH (150.0 mL) and H₂O (30.0 mL) was added NaBH₄ (6.88 g, 181.80 mmol) in portions at −5° C. under a nitrogen atmosphere. The resulting mixture was stirred for 2 h at 0-10° C. Desired product could be detected by LCMS. The reaction was quenched with brine (100 ml) at 0° C. The resulting mixture was extracted with pet. ether (100 mL). The aqueous layer was separated and filtered. The filter cake was washed with MeOH (2×50 mL). The filtrate was concentrated under reduced pressure to afford (5-bromo-1-methyl-1,2,4-triazol-3-yl)methanol (6 g, crude) as a light yellow solid. LCMS (ESI): m/z: [M+H] calc'd for C₄H₆BrN₃O 191.98; found 192.0.

Step 3. A solution of (5-bromo-1-methyl-1,2,4-triazol-3-yl)methanol (6.0 g) and HBr in AcOH (144.0 mL) was stirred for overnight at 80° C. The mixture was neutralized to pH 9 with saturated NaHCO₃ (aq.). The resulting mixture was extracted with EtOAc (3×60 mL). The combined organic layers were washed with brine (3×100 ml), dried over anhydrous Na₂SO₄. After filtration, the filtrate was concentrated under reduced pressure. This resulted in 5-bromo-3-(bromomethyl)-1-methyl-1,2,4-triazole (6 g, crude) as a white solid. LCMS (ESI): m/z: [M+H] calc'd for C₄H₅Br₂N₃ 253.89; found 253.8.

Step 4. To a stirred mixture of 5-bromo-3-(bromomethyl)-1-methyl-1,2,4-triazole (6.0 g, 23.54 mmol) and tert-butyl 2-[(diphenylmethylidene)amino]acetate (6.95 g, 23.54 mmol) in toluene (42 mL) and DCM (18.0 mL) was added (2R,4R,5S)-1-(anthracen-9-ylmethyl)-5-ethenyl-2-[(S)-(prop-2-en-1-yloxy)(quinolin-4-yl)methyl]-1-azabicyclo[2.2.2]octan-1-ium bromide (1.43 g, 2.35 mmol) in portions at 0° C. under argon atmosphere. The resulting mixture was stirred and KOH (60 mL) in H₂O was added. The resulting mixture was stirred for 24 h at −10° C. under an argon atmosphere. Desired product could be detected by LCMS. The reaction was quenched with sat. NH₄Cl (aq.) at 0° C. The resulting mixture was extracted with EtOAc (3×100 mL). The combined organic layers were washed with brine (1×200 mL), dried over anhydrous Na₂SO₄. After filtration, the filtrate was concentrated under reduced pressure. The residue was purified by Prep-TLC to afford tert-butyl (2S)-3-(5-bromo-1-methyl-1,2,4-triazol-3-yl)-2-[(diphenylmethylidene)amino]propanoate (5 g, 38.6% yield) as a yellow oil. LCMS (ESI): m/z: [M+H] calc'd for C₂₁H₂₁BrN₄O₂ 469.12; found 469.1.

Step 5. To a stirred solution of tert-butyl (2S)-3-(5-bromo-1-methyl-1,2,4-triazol-3-yl)-2-[(diphenylmethylidene)amino]propanoate (5.0 g, 10.65 mmol) in DCM (50.0 mL) was added TFA (25.0 mL) dropwise at 0° C. under argon atmosphere. The resulting mixture was stirred for 16 h at room temperature under an argon atmosphere. The resulting mixture was concentrated under reduced pressure to afford (2S)-2-amino-3-(5-bromo-1-methyl-1,2,4-triazol-3-yl)propanoic acid (6 g, crude) as a brown oil. LCMS (ESI): m/z: [M+H] calc'd for C₆H₉BrN₄O₂ 249.00; found 249.0.

Step 6. To a stirred solution of (2S)-2-amino-3-(5-bromo-1-methyl-1,2,4-triazol-3-yl)propanoic acid (6.0 g, 24.09 mmol) in THF (36.0 mL) was added NaHCO₃ (10.14 g, 120.69 mmol), Boc₂O (7.89 g, 36.14 mmol) in portions at 0° C. under an argon atmosphere. The resulting mixture was stirred for 16 h at room temperature. Desired product could be detected by LCMS. The resulting mixture was concentrated under reduced pressure. The mixture was purified by reverse phase chromatography to afford (2S)-3-(5-bromo-1-methyl-1,2,4-triazol-3-yl)-2-[(tert-butoxycarbonyl)amino]propanoic acid (3 g, 33.9% yield) as a white solid. LCMS (ESI): m/z: [M+H] calc'd for C₁₁H₁₇BrN₄O₄ 349.05; found 349.0.

Step 7. To a stirred solution of 2-[[(2M)-1-ethyl-2-[2-[(1S)-1-methoxyethyl]pyridin-3-yl]-5-(4,4,5,5-tetramethyl-1,3,2-dioxaborolan-2-yl)indol-3-yl]methyl]-2-methylpropyl (3S)-1,2-diazinane-3-carboxylate (1.0 g, 1.65 mmol) in DMF (10.0 mL) was added DIPEA (4.28 g, 33.08 mmol), (2S)-3-(5-bromo-1-methyl-1,2,4-triazol-3-yl)-2-[(tert-butoxycarbonyl)amino]propanoic acid (0.69 g, 1.98 mmol) and HATU (0.75 g, 1.99 mmol) in portions at 0° C. The resulting mixture was stirred for 2 h at 20° C. under an argon atmosphere. Desired product could be detected by LCMS. The resulting mixture was quenched with H₂O (100 mL) and extracted with EtOAc (3×30 mL). The combined organic layers were washed with brine (3×100 mL), dried over anhydrous Na₂SO₄. After filtration, the filtrate was concentrated under reduced pressure. The residue was purified by reverse phase chromatography to afford 2-[[(2M)-1-ethyl-2-[2-[(1S)-1-methoxyethyl]pyridin-3-yl]-5-(4,4,5,5-tetramethyl-1,3,2-dioxaborolan-2-yl)indol-3-yl]methyl]-2-methylpropyl (3S)-1-[(2S)-3-(5-bromo-1-methyl-1,2,4-triazol-3-yl)-2-[(tert-butoxycarbonyl)amino]propanoyl]-1,2-diazinane-3-carboxylate (800 mg, 46.5% yield) as a light yellow solid. LCMS (ESI): m/z: [M+H] calc'd for C₄₅H₆₄BBrN₈O₈ 935.42; found 935.2.

Step 8. To a stirred solution of 2-[[(2M)-1-ethyl-2-[2-[(1S)-1-methoxyethyl]pyridin-3-yl]-5-(4,4,5,5-tetramethyl-1,3,2-dioxaborolan-2-yl)indol-3-yl]methyl]-2-methylpropyl (3S)-1-[(2S)-3-(5-bromo-1-methyl-1,2,4-triazol-3-yl)-2-[(tert-butoxycarbonyl)amino]propanoyl]-1,2-diazinane-3-carboxylate (800.0 mg, 0.86 mmol) in dioxane (10.0 mL) were added K₃PO₄ (0.45 g, 2.12 mmol), XPhos (122.26 mg, 0.27 mmol), XPhos Pd G3 (0.22 g, 0.27 mmol) and H₂O (2.0 mL) at room temperature. The resulting mixture was stirred for 3 h at 75° C. under an argon atmosphere. Desired product could be detected by LCMS. The resulting mixture was extracted with EtOAc (3×100 mL). The combined organic layers were washed with brine (3×60 mL), dried over anhydrous Na₂SO₄. After filtration, the filtrate was concentrated under reduced pressure. The residue was purified by reverse phase chromatography to afford tert-butyl ((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)carbamate (400 mg, 56.8% yield) as a light yellow solid. LCMS (ESI): m/z: [M+H] calc'd for C₃₉H₅₂N₈O₆ 729.41; found 729.3.

Step 9. To a solution of tert-butyl ((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)carbamate (400.0 mg, 0.56 mmol) in DCM (1 mL) was added TFA (0.5 mL). The reaction was stirred for 1 h at room temperature under an argon atmosphere. After concentration, the mixture was neutralized to pH 8 with saturated NaHCO₃ (aq., 20 mL). The mixture was extracted with DCM (3×20 mL). The organic layers were dried over Na₂SO₄ and concentrated to afford (6³S,4S,Z)-4-amino-1¹-ethyl-1²-(2-((S)-1-methoxyethyl)pyridin-3-yl)-2¹,10,10-trimethyl-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-5,7-dione (500 mg, crude) as a light yellow solid. ESI-MS m/z=629.3 [M+H]+; Calculated MW: 628.3. LCMS (ESI): m/z: [M+H] calc'd for C₃₄H₄₄N₈O₄ 629.36; found 629.3.

Step 10. To a stirred solution of (6³S,4S,Z)-4-amino-1¹-ethyl-1²-(2-((S)-1-methoxyethyl)pyridin-3-yl)-2¹,10,10-trimethyl-6¹,6²,6³,6⁴,6⁵,6⁶-hexahydro-1¹H,2¹H-8-oxa-1(5,3)-indola-6(1,3)-pyridazina-2(5,3)- triazolacycloundecaphane-5,7-dione (170.0 mg, 0.27 mmol) and (R)-2-(((1-benzhydrylazetidin-3-yl)oxy)methyl)-3-methylbutanoic acid (114.68 mg, 0.32 mmol) in DMF (5 mL) were added DIPEA (698.86 mg, 5.41 mmol) and HATU (123.36 mg, 0.32 mmol) dropwise at 0° C. under an air atmosphere. The resulting mixture was stirred for 2 h at 0° C. The resulting mixture was diluted with 25 mL H₂O. The resulting mixture was extracted with EtOAc (3×25 mL). The combined organic layers were washed with brine (3×25 mL), dried over anhydrous Na₂SO₄. After filtration, the filtrate was concentrated under reduced pressure to afford (2R)-2-(((1-benzhydrylazetidin-3-yl)oxy)methyl)-N-((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)-3-methylbutanamide (180 mg, crude) as an off-white oil. LCMS (ESI): m/z: [M+H] calc'd for C₅₆H₆₉N₉O₆ 964.54; found 964.4.

Step 11. To a stirred solution of (2R)-2-(((1-benzhydrylazetidin-3-yl)oxy)methyl)-N-((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)-3-methylbutanamide (180.0 mg, 0.19 mmol) and Pd/C (90.0 mg, 0.85 mmol) in MeOH (10 mL) was added Boc₂O (81.48 mg, 0.37 mmol) at room temperature under a hydrogen atmosphere. The resulting mixture was stirred overnight at room temperature. The resulting mixture was filtered, the filter cake was washed with MeOH (3×10 mL). The filtrate was concentrated under reduced pressure to afford tert-butyl 3-((2R)-2-(((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)carbamoyl)-3-methylbutoxy)azetidine-1-carboxylate (80 mg, 47.7% yield) as an off-white solid. LCMS (ESI): m/z: [M+H] calc'd for C₄₈H₆₇N₉O₈ 898.52; found 898.4.

Step 12. To a stirred solution of tert-butyl 3-((2R)-2-(((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)carbamoyl)-3-methylbutoxy)azetidine-1-carboxylate in DCM (2 mL) was added TFA (1.0 mL) dropwise at 0° C. under an air atmosphere. The resulting mixture was stirred for 1 h at 0° C. The resulting mixture was concentrated under reduced pressure to afford (2R)-2-((azetidin-3-yloxy)methyl)-N-((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)-3-methylbutanamide (85 mg, crude) as a yellow green oil.

Step 13. To a stirred solution of (2R)-2-((azetidin-3-yloxy)methyl)-N-((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)-3-methylbutanamide (80.0 mg, 0.10 mmol) and 4-(dimethylamino)-4-methylpent-2-ynoic acid (38.90 mg, 0.25 mmol) in DMF (2 mL) were added DIPEA (518.27 mg, 4.01 mmol) and COMU (51.52 mg, 0.12 mmol) in portions at 0° C. The reaction mixture was stirred under an air atmosphere for 2 h. The crude product (150 mg) was purified by reverse phase chromatography to afford (2R)-2-(((1-(4-(dimethylamino)-4-methylpent-2-ynoyl)azetidin-3-yl)oxy)methyl)-N-((6³S,4S,Z)-1¹-ethyl-1²-(2-((S)-1-methoxyethyl)pyridin-3-yl)-2¹,10,10-trimethyl-5,7-dioxo-6¹,6²,6³,6⁴,6⁵,6⁶-hexahydro-1¹H,2¹H-8-oxa-1(5,3)-indola-6(1,3)-pyridazina-2(5,3)-triazolacycloundecaphane-4-yl)-3-methylbutanamide (15.3 mg, 16.3% yield) as an off-white solid. ¹H NMR (400 MHz, DMSO-d₆) δ 8.77 (dd, J=4.8, 1.7 Hz, 1H), 8.15 (d, J=1.7 Hz, 1H), 8.07 (d, J=8.1 Hz, 1H), 7.85-7.78 (m, 1H), 7.70 (d, J=8.6 Hz, 1H), 7.58-7.48 (m, 2H), 5.82 (s, 1H), 4.95 (d, J=11.7 Hz, 1H), 4.41-4.30 (m, 5H), 4.30 (d, J=8.2 Hz, 2H), 4.25 (d, J=5.6 Hz, 4H), 4.10 (td, J=17.1, 16.1, 9.1 Hz, 2H), 3.99-3.82 (m, 3H), 3.71-3.60 (m, 1H), 3.54-3.43 (m, 3H), 3.39 (s, 2H), 3.22 (d, J=1.6 Hz, 1H), 2.92 (d, J=13.6 Hz, 1H), 2.86-2.77 (m, 2H), 2.45 (s, 6H), 2.37 (q, J=7.7 Hz, 1H), 2.17 (d, J=6.6 Hz, 2H), 2.03 (d, J=10.2 Hz, 2H), 1.78-1.66 (m, 3H), 1.47 (t, J=10.9 Hz, 6H), 1.35-1.28 (m, 12H), 0.32 (s, 3H). LCMS (ESI): m/z: [M+H] calc'd for C₅₁H₇₀N₁₀O₇ 935.55; found 935.3.

### Example A692. Synthesis of (3S)-1-(4-(dimethylamino)-4-methylpent-2-ynoyl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)amino)-3-methyl-1-oxobutan-2-yl)-N-methylpyrrolidine-3-carboxamide

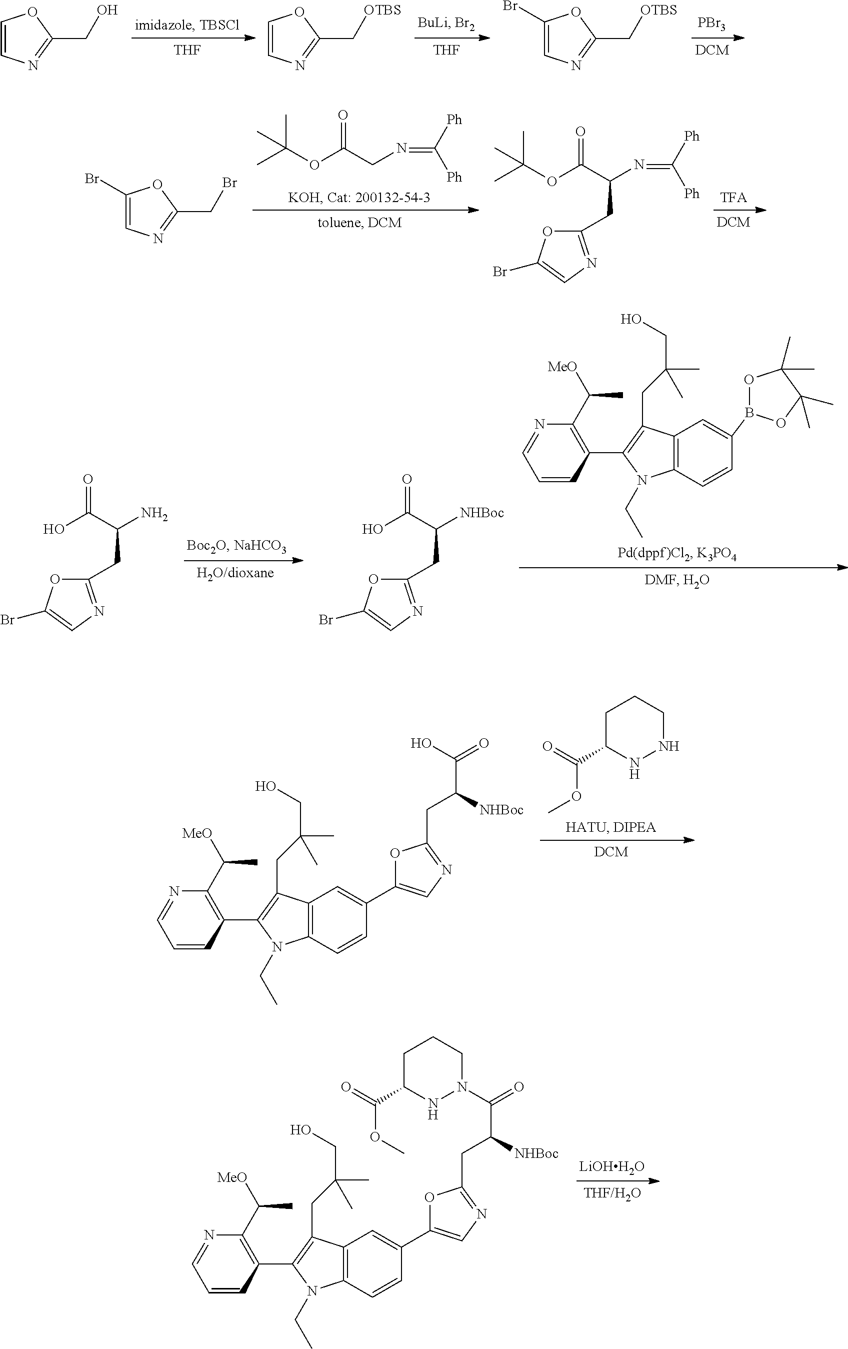

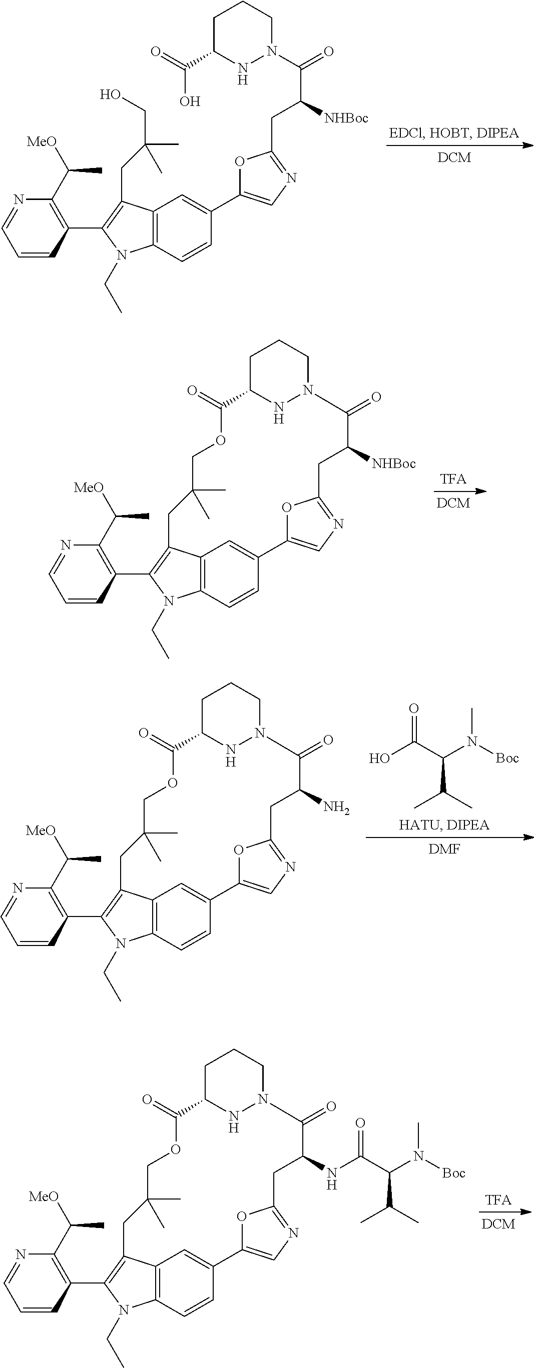

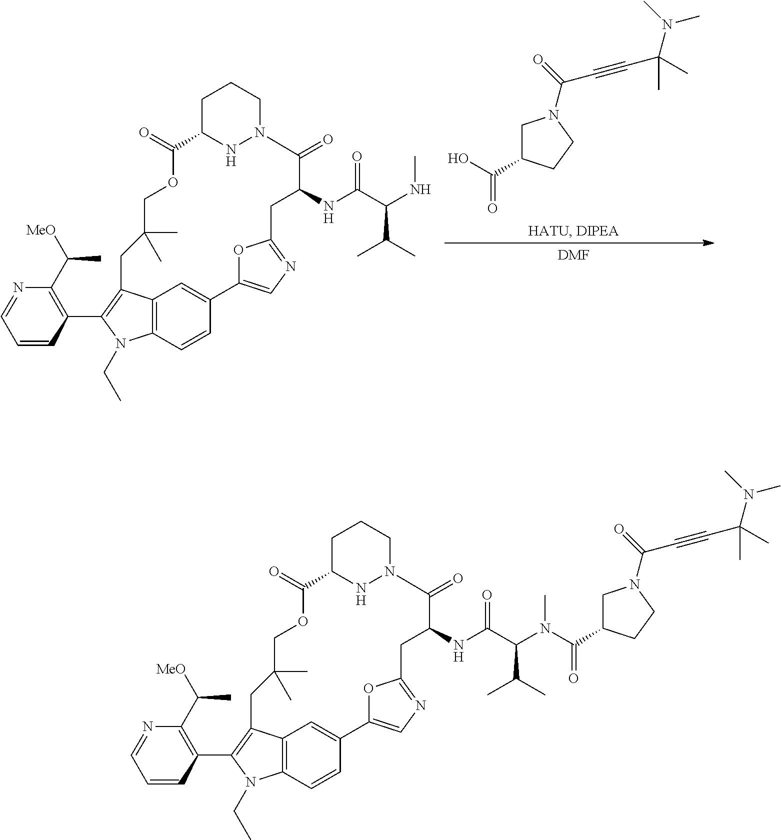

Step 1. To a solution of 1,3-oxazol-2-ylmethanol (5.0 g, 50.46 mmol) in THF (75 mL), were added imidazole (8.59 mg, 0.13 mmol), and TBSCI (11.41 mg, 0.08 mmol) at 0° C. The resulting solution was stirred for 5 h then concentrated under reduced pressure. The crude material was purified by silica gel column chromatography to afford 2-[[(tert-butyldimethylsilyl)oxy]methyl]-1,3-oxazole (10 g, 92.8% yield) as colorless oil. LCMS (ESI): m/z: [M+H] calc'd for C₁₀H₁₉NO₂Si 214.13; found 214.3.

Step 2. To a solution of 2-[[(tert-butyldimethylsilyl)oxy]methyl]-1,3-oxazole (10.0 g, 46.87 mmol) in THF (150.0 mL, 1851.45 mmol) at −78° C. was added n-BuLi (22.4 mL, 56.25 mmol) over 10 min and stirred for 30 min at −78° C. under an argon atmosphere. Then the solution of Br₂ (3.6 mL, 70.31 mmol) in THF (10 mL) was added over 10 min to the solution at −78° C. The resulting solution was slowly warmed to room temperature and stirred for 2 h. The resulting mixture was diluted with NH₄Cl/H₂O (100 mL) and extracted with EtOAc (3×100 mL). The combined organic layers were dried over anhydrous Na₂SO₄. After filtration, the filtrate was concentrated under reduced pressure and purified by silica gel column chromatography to afford 5-bromo-2-[[(tert-butyldimethylsilyl)oxy]methyl]-1,3-oxazole (5.3 g, 38.6% yield) as yellow oil. LCMS (ESI): m/z: [M+H] calc'd for C₁₀H₁₈BrNO₂Si 292.04; found 292.0.

Step 3. To a solution of 5-bromo-2-[[(tert-butyldimethylsilyl)oxy]methyl]-1,3-oxazole (4.0 g, 13.69 mmol) in DCM (60.0 mL) was added PBr₃ (7.41 g, 27.37 mmol) at 0° C. under an argon atmosphere. The resulting solution was stirred for 4 h then diluted with NaHCO₃/H₂O (30 mL). The mixture was extracted with EtOAc (3×40 mL). The organic layers were concentrated under reduced pressure and purified by silica gel column chromatography to afford 5-bromo-2-(bromomethyl)-1,3-oxazole (2.5 g, 75.7% yield) as yellow oil. LCMS (ESI): m/z: [M+H] calc'd for C₄H₃Br₂NO 239.87; found 241.9.

Step 4. A mixture of 5-bromo-2-(bromomethyl)-1,3-oxazole (9.0 g, 37.36 mmol), Cat: 200132-54-3 (2.26 g, 3.74 mmol), DCM (45.0 mL), toluene (90.0 mL), KOH (20.96 g, 373.63 mmol), H₂O (42 mL), and tert-butyl 2-[(diphenylmethylidene)amino]acetate (13.24 g, 44.82 mmol) at 0° C. was stirred for 4 h then diluted with H₂O (30 mL). The mixture was extracted with DCM (3×40 mL). The organic layers were concentrated under reduced pressure and purified by reverse phase column chromatography to afford tert-butyl (2S)-3-(5-bromo-1,3-oxazol-2-yl)-2-[(diphenylmethylidene)amino]propanoate (4.8 g, 28.2% yield) as a yellow solid. LCMS (ESI): m/z: [M+H] calc'd for C₂₃H₂₃BrN₂O₃ 455.10; found 457.1.

Step 5. A mixture of tert-butyl (2S)-3-(5-bromo-1,3-oxazol-2-yl)-2-[(diphenylmethylidene)amino]propanoate (1.20 g, 2.64 mmol), DCM (10.0 mL, 157.30 mmol), and TFA (5.0 mL, 67.32 mmol) at 0° C. was stirred for 2 h then concentrated under reduced pressure to afford (S)-3-(5-bromooxazol-2-yl)-2-((2,2,2-trifluoroacetyl)-14-azaneyl)propanoic acid (0.5 g, 81.3% yield) as a yellow solid. LCMS (ESI): m/z: [M+H] calc'd for C₆H₇BrN₂O₃ 234.97; found 237.0.

Step 6. A mixture of (S)-3-(5-bromooxazol-2-yl)-2-((2,2,2-trifluoroacetyl)-14-azaneyl)propanoic acid (500.0 mg, 2.13 mmol), Boc₂O (928.56 mg, 4.26 mmol), dioxane (2.50 mL), H₂O (2.50 mL), and NaHCO₃ (714.84 mg, 8.51 mmol) at 0° C. was stirred for 3 h. The resulting solution was purified by reverse phase column chromatography to afford (2S)-3-(5-bromo-1,3-oxazol-2-yl)-2-[(tert-butoxycarbonyl)amino]propanoic acid (0.65 g, 91.1% yield) as a yellow solid. LCMS (ESI): m/z: [M+H] calc'd for C₁₁H₁₅BrN₂O₅ 335.02; found 334.8.

Step 7. To a solution of (2S)-3-(5-bromo-1,3-oxazol-2-yl)-2-[(tert-butoxycarbonyl)amino]propanoic acid (500.0 mg, 1.49 mmol) and 3-[(2M)-1-ethyl-2-[2-[(1S)-1-methoxyethyl]pyridin-3-yl]-5-(4,4,5,5-tetramethyl-1,3,2-dioxaborolan-2-yl)indol-3-yl]-2,2-dimethylpropan-1-ol (808.16 mg, 1.64 mmol) in DMF (5.0 mL) and H₂O (1.0 mL) were added K₃PO₄ (791.67 mg, 3.73 mmol) and Pd(dppf)Cl₂ (109.16 mg, 0.15 mmol). The resulting mixture was stirred for 2 h at 70° C. under an argon atmosphere. The mixture was purified by reverse phase column chromatography to afford (2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoic acid (600 mg, 64.79% yield) as a light brown solid. LCMS (ESI): m/z: [M+H] calc'd for C₃₄H₄₄N₄O₇ 621.33; found 621.3.

Step 8. To a stirred mixture of methyl (3S)-1,2-diazinane-3-carboxylate (627.10 mg, 4.350 mmol) and (2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoic acid (900.0 mg, 1.45 mmol) in DCM (10.0 mL) were added HATU (661.54 mg, 1.74 mmol) and DIPEA (3747.71 mg, 29.00 mmol) at 0° C. The resulting mixture was stirred for 2 h. Desired product could be detected by LCMS. The resulting mixture was concentrated under reduced pressure and the crude material was purified by silica gel column chromatography to afford methyl (3S)-1-[(2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylate (900 mg, 83.11%) as a brown yellow solid. LCMS (ESI): m/z: [M+H] calc'd for C₄₀H₅₄N₆O₈ 747.41; found 747.2.

Step 9. To a stirred mixture of methyl (3S)-1-[(2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylate (2000.0 mg, 2.68 mmol) in THF (18. mL) and H₂O (6.0 mL) was added LiOH·H₂O (337.10 mg, 8.03 mmol) at 0° C. The resulting mixture was stirred for 2 h. Desired product could be detected by LCMS. The reaction was quenched with H₂O at 0° C. and adjusted to pH 6 with 1N HCl solution. The resulting mixture was extracted with EtOAc (3×50 mL). The combined organic layers were washed with brine (1×10 mL), dried over anhydrous Na₂SO₄. After filtration, the filtrate was concentrated under reduced pressure to afford (3S)-1-[(2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylic acid (1300 mg, 66.2% yield) as a yellow solid. LCMS (ESI): m/z [M+H] calc'd for C₃₉H₅₂N₆O₈ 733.39; found 733.3.

Step 10. To a stirred mixture of (3S)-1-[(2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylic acid (1.2 g, 1.64 mmol) and DIPEA (8.5 g, 65.50 mmol) in DCM (120.0 mL) were added HOBT (1.8 g, 13.10 mmol) and EDCI (7.8 g, 40.93 mmol) at 0° C. The resulting mixture was stirred for 2 h. Desired product could be detected by LCMS. The mixture was concentrated under reduced pressure and purified by silica gel column chromatography to afford tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)carbamate (660 mg, 56.4% yield) as a brown yellow solid. LCMS (ESI): m/z: [M+H] calc'd for C₃₉H₅₀N₆O₇ 715.38; found 715.3.

Step 11. A mixture of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)carbamate (20.0 mg, 0.028 mmol) and TFA (3.0 mL) in DCM (6.0 mL) at 0° C. was stirred for 2 h. Desired product could be detected by LCMS. The resulting mixture was concentrated under reduced pressure to afford (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(5,2)-oxazola-1(5,3)-indola-6(1,3)-pyridazinacycloundecaphane-5,7-dione (160 mg, crude) as a yellow green solid. LCMS (ESI): m/z: [M+H] calc'd for C₃₄H₄₂N₆O₅ 615.33; found 615.2.

Step 12. To a stirred mixture of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(5,2)-oxazola-1(5,3)-indola-6(1,3)-pyridazinacycloundecaphane-5,7-dione (180.0 mg, 0.293 mmol) and (2S)-2-[(tert-butoxycarbonyl)(methyl)amino]-3-methylbutanoic acid (135.45 mg, 0.59 mmol) in DMF (2.0 mL) were added HATU (133.60 mg, 0.35 mmol) and DIPEA (756.86 mg, 5.86 mmol) at 0° C. The resulting mixture was stirred for 2 h. Desired product could be detected by LCMS. The mixture was purified by reverse phase column chromatography to afford tert-butyl ((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)amino)-3-methyl-1-oxobutan-2-yl)(methyl)carbamate (160 mg, 66% yield) as a brown yellow solid. LCMS (ESI): m/z: [M+H] calc'd for C₄₅H₆₁N₇O₈ 828.47; found 828.4.

Step 13. To a stirred mixture of tert-butyl ((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)amino)-3-methyl-1-oxobutan-2-yl)(methyl)carbamate (160.0 mg, 0.19 mmol) in DCM (2.0 mL) was added TFA (1.0 mL) at 0° C. The resulting mixture was stirred for 2 h. Desired product could be detected by LCMS. The resulting mixture was concentrated under reduced pressure to afford (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)-3-methyl-2-(methylamino)butanamide (130 mg, crude) as a yellow solid. LCMS (ESI): m/z: [M+H] calc'd for C₄₀H₅₃N₇O₆ 728.41; found 728.5.

Step 14. To a stirred mixture of ((2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)- pyridazinacycloundecaphane-4-yl)-3-methyl-2-(methylamino)butanamide (130.0 mg, 0.18 mmol) and (3S)-1-[4-(dimethylamino)-4-methylpent-2-ynoyl]pyrrolidine-3-carboxylic acid (180.25 mg, 0.72 mmol) in DMF (2.0 mL) were added DIPEA (461.64 mg, 3.57 mmol) and HATU (135.81 mg, 0.36 mmol) at 0° C. The resulting mixture was stirred for 2 h. Desired product could be detected by LCMS. The mixture was purified by reverse phase column chromatography to afford (3S)-1-(4-(dimethylamino)-4-methylpent-2-ynoyl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,2)-oxazola-1(5,3)-indola-6(1,3)-pyridazinacycloundecaphane-4-yl)amino)-3-methyl-1-oxobutan-2-yl)-N-methylpyrrolidine-3-carboxamide (55.3 mg, 31.3% yield) as a solid. ¹H NMR (400 MHz, DMSO-d₆) δ 8.77 (dd, J=4.8, 1.7 Hz, 1H), 8.09-8.00 (m, 1H), 7.92 (s, 1H), 7.88-7.80 (m, 1H), 7.62 (d, J=8.7 Hz, 1H), 7.59-7.49 (m, 2H), 7.39-7.30 (m, 1H), 5.69 (p, J=8.8 Hz, 1H), 5.44 (d, J=12.1 Hz, 1H), 4.67 (d, J=10.7 Hz, 1H), 4.30-4.15 (m, 3H), 3.99 (dt, J=13.2, 6.4 Hz, 3H), 3.89-3.79 (m, 1H), 3.62 (ddd, J=30.5, 18.6, 11.4 Hz, 5H), 3.39 (dd, J=9.4, 3.8 Hz, 2H), 3.21-3.13 (m, 1H), 3.08 (d, J=15.0 Hz, 3H), 2.99-2.74 (m, 6H), 2.26-2.18 (m, 5H), 2.16 (s, 2H), 2.14-1.94 (m, 3H), 1.86-1.68 (m, 2H), 1.57 (q, J=9.2, 5.8 Hz, 1H), 1.44-1.27 (m, 9H), 0.94 (d, J=6.6 Hz, 4H), 0.89 (dd, J=6.5, 2.5 Hz, 2H), 0.80 (d, J=6.3 Hz, 2H), 0.77-0.69 (m, 4H), 0.58 (d, J=20.4 Hz, 3H). LCMS (ESI): m/z: [M+H] calc'd for C₅₃H₇₁N₉O₈ 962.55; found 962.5.

### Example A675. Synthesis of (2R)-2-(((1-(4-(dimethylamino)-4-methylpent-2-ynoyl)azetidin-3-yl)oxy)methyl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)-3-methylbutanamide

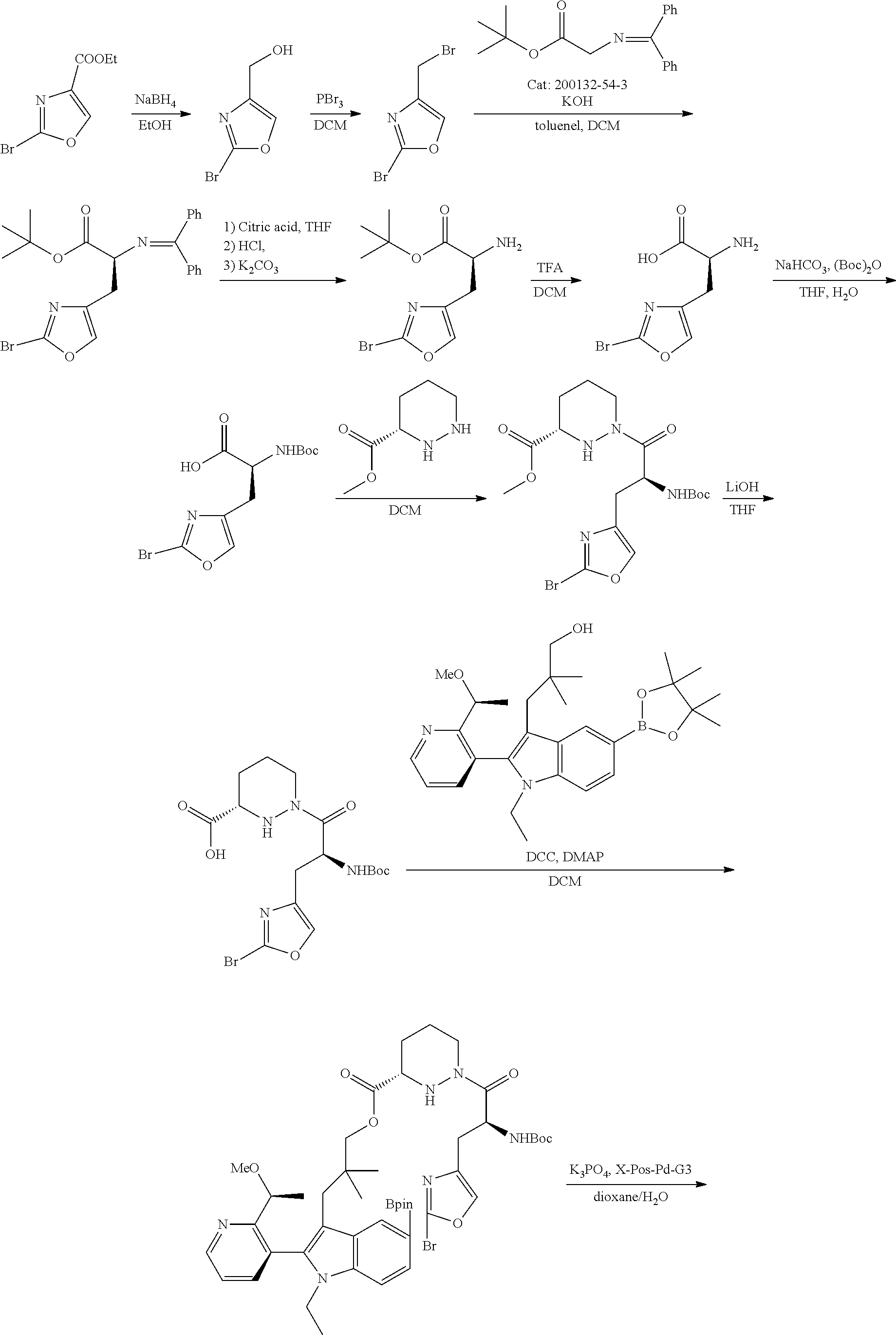

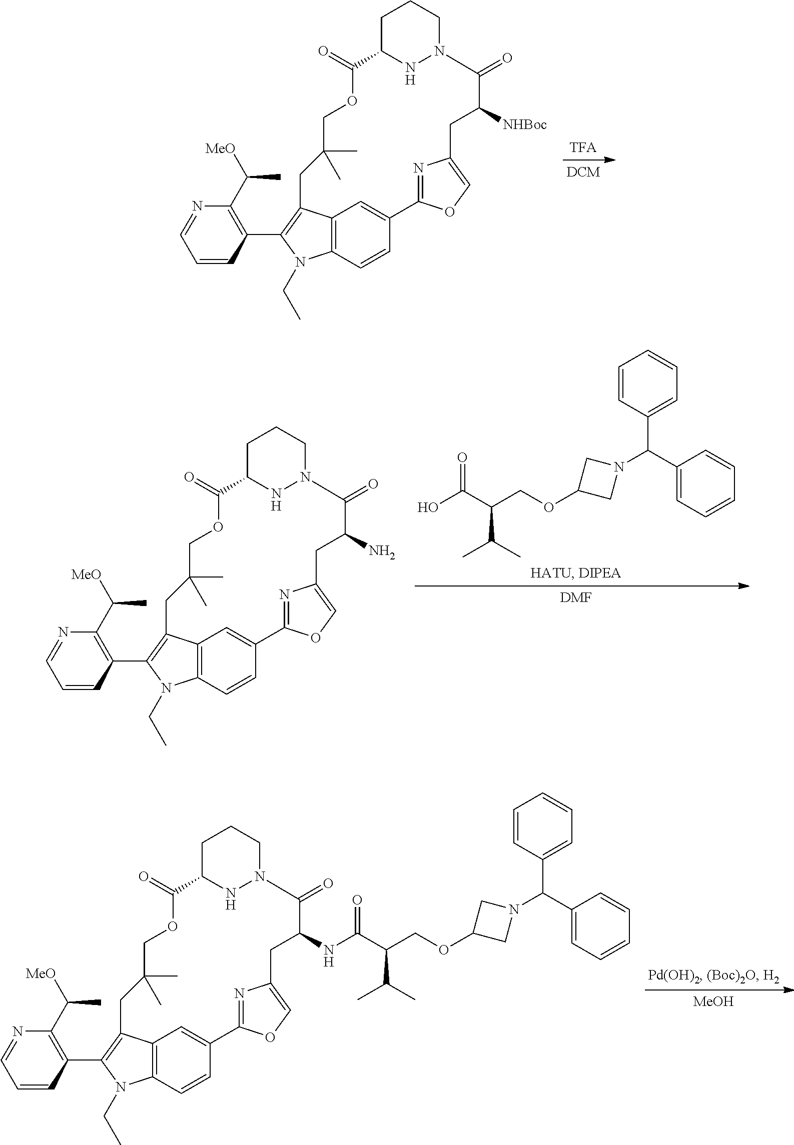

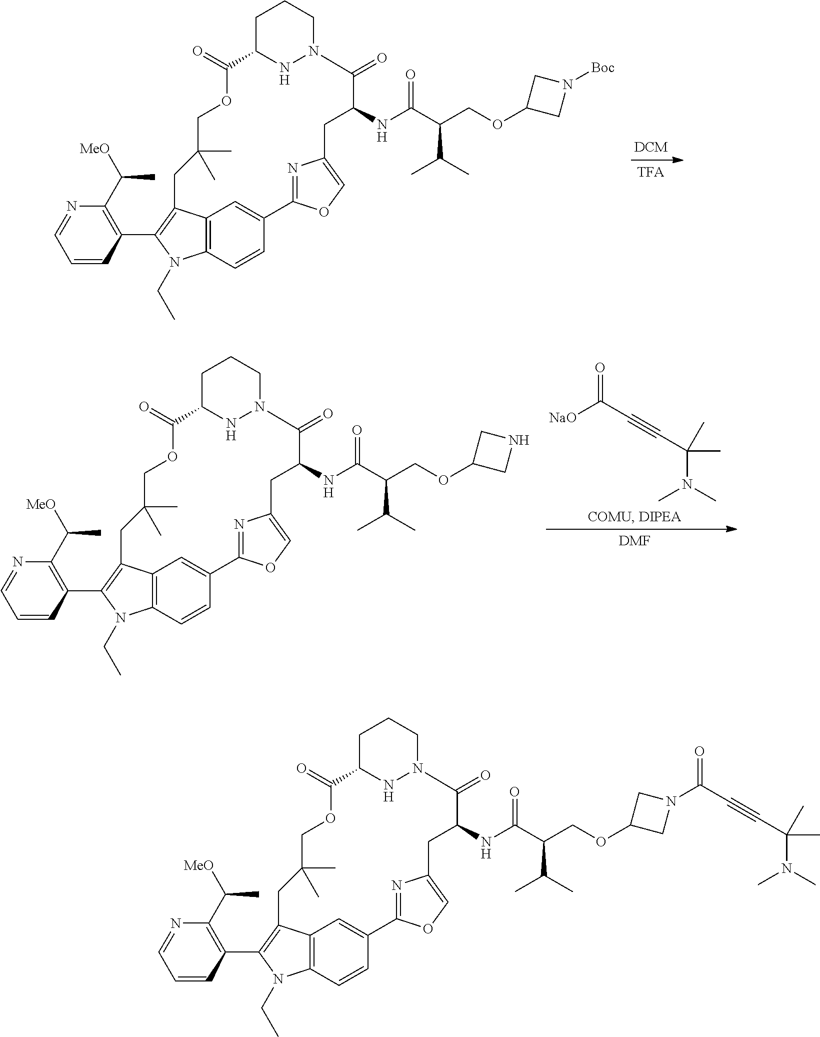

Step 1. A mixture of 2-bromo-4-(ethoxycarbonyl)-1,3-oxazol-5-ylium (6.83 g, 31.19 mmol), EtOH (100.0 mL) and NaBH₄ (4.72 g, 124.76 mmol) at 0° C. was stirred for 6 h at 0° C. under an air atmosphere. The reaction was quenched with H₂O at 0° C. The resulting mixture was extracted with EtOAc (3×100 mL). The combined organic layers were dried over anhydrous Na₂SO₄. After filtration, the filtrate was concentrated under reduced pressure to afford (2-bromo-1,3-oxazol-4-yl)methanol (4.122 g, 74.3% yield) as a solid. LCMS (ESI): m/z: [M+H] calc'd for C₄H₄BrNO₂ 177.95; found 178.0.

Step 2. A mixture of (2-bromo-1,3-oxazol-4-yl)methanol (4.30 g, 24.16 mmol), DCM (50 mL) and phosphorus tribromide (9809.39 mg, 36.24 mmol) at 0° C. was stirred overnight at 0° C. under an air atmosphere. The reaction was quenched by the addition of NaHCO₃ (aq.) at 0° C. The resulting mixture was extracted with EtOAc (3×100 mL). The combined organic layers were dried over anhydrous Na₂SO₄. After filtration, the filtrate was concentrated under reduced pressure to afford 2-bromo-4-(bromomethyl)-1,3-oxazole (3.28 g, 56.4% yield) as a liquid. LCMS (ESI): m/z: [M+H] calc'd for C₄H₃Br₂NO 239.87; found 239.9.

Step 3. A mixture of 2-bromo-4-(bromomethyl)-1,3-oxazole (3280.0 mg, 13.62 mmol), KOH (9M, 10 mL), 30 mL mixture of toluene/DCM (7/3) and tert-butyl 2-[(diphenylmethylidene)amino]acetate (5228.74 mg, 17.70 mmol) at −16° C. was stirred overnight under an air atmosphere. The resulting mixture was extracted with EtOAc (3×50 mL). The combined organic layers were dried over anhydrous Na₂SO₄. After filtration, the filtrate was concentrated under reduced pressure to afford tert-butyl (2S)-3-(2-bromo-1,3-oxazol-4-yl)-2-[(diphenylmethylidene)amino]propanoate (8.33 g, 80.6% yield) as a solid. LCMS (ESI): m/z: [M+H] calc'd for C₂₃H₂₃BrN₂O₃ 455.10; found 455.1.

Step 4. A mixture of tert-butyl (2S)-3-(2-bromo-1,3-oxazol-4-yl)-2-[(diphenylmethylidene)amino]propanoate (4100.0 mg, 9.0 mmol) and citric acid (1 N) (40.0 mL, 0.21 mmol), in THF (40 mL) at room temperature was stirred overnight under an air atmosphere. The reaction was quenched by the addition of HCl (aq.) (100 mL) at 0° C. The aqueous layer was extracted with EtOAc (3×100 mL). K₂CO₃ (aq.) (200 mL) was added to the resulting mixture and extracted with EtOAc (3×100 mL). The organic layer was concentrated under reduced pressure to afford tert-butyl (2S)-2-amino-3-(2-bromo-1,3-oxazol-4-yl)propanoate (1730 mg, 66% yield) as a dark yellow solid. LCMS (ESI): m/z: [M+H] calc'd for C₁₀H₁₅BrN₂O₃ 291.03; found 291.0.

Step 5. A mixture of tert-butyl (2S)-2-amino-3-(2-bromo-1,3-oxazol-4-yl)propanoate (1780.0 mg, 6.11 mmol), TFA (10.0 mL) and DCM (10.0 mL) at 0° C. was stirred for overnight under an air atmosphere. The resulting mixture was concentrated under reduced pressure to afford (2S)-2-amino-3-(2-bromo-1,3-oxazol-4-yl)propanoic acid (1250 mg, 87% yield) as a dark yellow solid. LCMS (ESI): m/z [M+H] calc'd for C₆H₇BrN₂O₃ 234.97; found 234.9.

Step 6. A mixture of di-tert-butyl dicarbonate (4178.58 mg, 19.15 mmol), THF (10 mL), H₂O (10 mL), (2S)-2-amino-3-(2-bromo-1,3-oxazol-4-yl)propanoic acid (1500.0 mg, 6.38 mmol) and NaHCO₃ (3216.74 mg, 38.29 mmol) at room temperature was stirred overnight under an air atmosphere. The reaction was quenched with H₂O at room temperature. The resulting mixture was concentrated under reduced pressure. The resulting mixture was extracted with EtOAc (3×100 mL). The aqueous layer was acidified to pH 6 with 1 M HCl (aq.). The resulting mixture was extracted with EtOAc (3×100 mL). The combined organic layers were dried over anhydrous Na₂SO₄. After filtration, the filtrate was concentrated under reduced pressure to afford (2S)-3-(2-bromo-1,3-oxazol-4-yl)-2-[(tert-butoxycarbonyl)amino]propanoic acid (920 mg, 43.0% yield) as an oil. LCMS (ESI): m/z: [M+H] calc'd for C₁₁H₁₅BrN₂O₅ 335.02; found 335.0.

Step 7. A mixture of 3-(2-bromo-1,3-oxazol-4-yl)-2-[(tert-butoxycarbonyl)amino]propanoic acid (850.0 mg, 2.54 mmol), methyl 1,2-diazinane-3-carboxylate (1.88 g, 13.04 mmol), DIPEA (1966.68 mg, 15.22 mmol), DCM (30.0 mL) and HATU (1446.48 mg, 3.80 mmol) at 0° C. was stirred for 3 h under an air atmosphere. The resulting mixture was extracted with DCM (3×50 mL). The combined organic layers were dried over anhydrous Na₂SO₄. After filtration, the filtrate was concentrated under reduced pressure. The resulting mixture was purified by reverse flash chromatography to afford methyl 1-[3-(2-bromo-1,3-oxazol-4-yl)-2-[(tert-butoxycarbonyl)amino]propanoyl]-1,2-diazinane-3-carboxylate (610 mg, 52.1% yield) as a solid. LCMS (ESI): m/z: [M+H] calc'd for C₁₇H₂₅BrN₄O₆ 461.10; found 461.0.

Step 8. A mixture of methyl 1-[3-(2-bromo-1,3-oxazol-4-yl)-2-[(tert-butoxycarbonyl)amino]propanoyl]-1,2-diazinane-3-carboxylate (570.0 mg, 1.24 mmol), LiOH (2.0 mL, 1 M aq.) and THF (2.0 mL) at 0° C. was stirred for 3 h under an air atmosphere. The resulting mixture was concentrated under reduced pressure. The resulting mixture was extracted with EtOAc (3×50 mL). The combined aqueous layers were acidified to pH 5 with 1N HCl (aq.). The aqueous phase was extracted with EtOAc (3×50 mL). The combined organic layers were dried over anhydrous Na₂SO₄. After filtration, the filtrate was concentrated under reduced pressure to afford 1-[3-(2-bromo-1,3-oxazol-4-yl)-2-[(tert-butoxycarbonyl)amino]propanoyl]-1,2-diazinane-3-carboxylic acid (500 mg, 90.5% yield) as a solid. LCMS (ESI): m/z: [M+H] calc'd for C₁₆H₂₃BrN₄O₆ 447.09; found 446.8.

Step 9. A mixture of 1-[3-(2-bromo-1,3-oxazol-4-yl)-2-[(tert-butoxycarbonyl)amino]propanoyl]-1,2-diazinane-3-carboxylic acid (450.0 mg, 1.01 mmol), 3-[(2M)-1-ethyl-2-[2-[(1S)-1-methoxyethyl]pyridin-3-yl]-5-(4,4,5,5-tetramethyl-1,3,2-dioxaborolan-2-yl)indol-3-yl]-2,2-dimethylpropan-1-ol (743.19 mg, 1.51 mmol), DMAP (24.58 mg, 0.20 mmol), DCM (15.0 mL) and DCC (311.37 mg, 1.51 mmol) at 0° C. was stirred for 3 h under an air atmosphere. The resulting mixture was extracted with EtOAc (3×20 mL). The combined organic layers were dried over anhydrous Na₂SO₄. After filtration, the filtrate was concentrated under reduced pressure. The residue was purified by Prep-TLC to afford 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl(S)-1-((S)-3-(2-bromooxazol-4-yl)-2-((tert-butoxycarbonyl)amino)propanoyl)hexahydropyridazine-3-carboxylate (330 mg, 35.6% yield) as a white solid. LCMS (ESI): m/z: [M+H] calc'd for C₄₅H₆₂BBrN₆O₉ 921.39; found 921.4.

Step 10. A mixture of 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl(S)-1-((S)-3-(2-bromooxazol-4-yl)-2-((tert-butoxycarbonyl)amino)propanoyl)hexahydropyridazine-3-carboxylate (290.0 mg, 0.33 mmol), K₃PO₄ (206.64 mg, 0.97 mmol), X-Phos (30.94 mg, 0.07 mmol), XPhos Pd G3 (54.93 mg, 0.07 mmol), dioxane (5. mL) and H₂O (1.0 mL) at 70° C. was stirred for 4 h under an argon atmosphere. The resulting mixture was extracted with EtOAc (3×20 mL). The combined organic layers were dried over anhydrous Na₂SO₄. After filtration, the filtrate was concentrated under reduced pressure. The residue was purified by Prep-TLC to afford tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)carbamate (130 mg, 56.0% yield) as a solid. LCMS (ESI): m/z: [M+H] calc'd for C₃₉H₅₀N₆O₇ 715.38; found 715.3.

Step 11. A mixture of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)carbamate (120.0 mg), DCM (2.0 mL) and TFA (0.2 mL) at room temperature was stirred for 6 h under an air atmosphere. The resulting mixture was concentrated under reduced pressure. to afford (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(2,4)-oxazola-1(5,3)-indola-6(1,3)-pyridazinacycloundecaphane-5,7-dione (90 mg, 87.2% yield) as a solid. LCMS (ESI): m/z: [M+H] calc'd for C₃₄H₄₂N₆O₅ 615.33; found 615.3.

Step 12. A mixture of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(2,4)-oxazola-1(5,3)-indola-6(1,3)-pyridazinacycloundecaphane-5,7-dione (200.0 mg, 0.33 mmol), (2R)-2-([[1-(diphenylmethyl)azetidin-3-yl]oxy]methyl)-3-methylbutanoic acid (172.49 mg, 0.49 mmol), DIPEA (420.48 mg, 3.25 mmol), DMF (3.0 mL) and HATU (148.44 mg, 0.39 mmol) at 0° C. was stirred for 3 h under an air atmosphere. The resulting mixture was extracted with EtOAc (3×20 mL). The combined organic layers were dried over anhydrous Na₂SO₄. After filtration, the filtrate was concentrated under reduced pressure. The residue was purified by Prep-TLC to afford (2R)-2-(((1-benzhydrylazetidin-3-yl)oxy)methyl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)-3-methylbutanamide (154 mg, 77.0% yield) as a solid. LCMS (ESI): m/z: [M+H] calc'd for C₅₆H₆₇N₇O₇ 950.52; found 950.6.

Step 13. A mixture of (2R)-2-(((1-benzhydrylazetidin-3-yl)oxy)methyl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)-3-methylbutanamide (240.0 mg, 0.25 mmol), (Boc)₂O (165.37 mg, 0.76 mmol), MeOH (5.0 mL) and Pd(OH)₂ (72.0 mg, 0.51 mmol) at room temperature was stirred overnight under an H₂ atmosphere. The resulting mixture was filtered, the filter cake was washed with MeOH (3×5 mL). The filtrate was concentrated under reduced pressure. The residue was purified by Prep-TLC to afford tert-butyl 3-((2R)-2-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)carbamoyl)-3-methylbutoxy)azetidine-1-carboxylate (150 mg, 67.2% yield) as a solid. LCMS (ESI): m/z: [M+H] calc'd for C₄₈H₆₅N₇O₉ 884.49; found 884.2.

Step 14. A mixture of tert-butyl 3-((2R)-2-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)- pyridazinacycloundecaphane-4-yl)carbamoyl)-3-methylbutoxy)azetidine-1-carboxylate (150.0 mg), DCM (2.0 mL) and TFA (0.40 mL) at 0° C. was stirred for 3 h under an air atmosphere. The resulting mixture was concentrated under reduced pressure to afford (2R)-2-((azetidin-3-yloxy)methyl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)-3-methylbutanamide (120 mg, 90.2% yield) as a solid. LCMS (ESI): m/z: [M+H] calc'd for C₄₃H₅₇N₇O₇ 784.44; found 784.2.

Step 15. A mixture of (2R)-2-((azetidin-3-yloxy)methyl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)-3-methylbutanamide (130.0 mg, 0.17 mmol), sodium 4-(dimethylamino)-4-methylpent-2-ynoate (44.07 mg, 0.25 mmol), DMF (3.0 mL), DIPEA (64.29 mg, 0.50 mmol) and COMU (106.46 mg, 0.25 mmol) at 0° C. was stirred for 3 h under an air atmosphere. The resulting mixture was extracted with EtOAc (3×20 mL). The combined organic layers were dried over anhydrous Na₂SO₄. After filtration, the filtrate was concentrated under reduced pressure. The crude product was purified by reverse phase chromatography to afford (2R)-2-(((1-(4-(dimethylamino)-4-methylpent-2-ynoyl)azetidin-3-yl)oxy)methyl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(2,4)-oxazola-1(5,3)-indola-6(1,3)-pyridazinacycloundecaphane-4-yl)-3-methylbutanamide (25 mg, 16.4% yield) as a solid. ¹H NMR (400 MHz, DMSO-d₆) δ 8.77 (dd, J=4.8, 1.8 Hz, 1H), 8.55 (s, 1H), 8.14 (dd, J=8.3, 2.9 Hz, 1H), 7.82 (d, J=7.6 Hz, 1H), 7.76-7.65 (m, 3H), 7.54 (dd, J=7.7, 4.7 Hz, 1H), 7.54-7.02 (m, 1H), 5.72 (td, J=7.4, 3.4 Hz, 1H), 4.97 (d, J=11.9 Hz, 1H), 4.46-4.24 (m, 6H), 4.18-4.04 (m, 2H), 3.94 (dd, J=32.9, 7.8 Hz, 1H), 3.77-3.63 (m, 2H), 3.57 (s, 1H), 3.49 (s, 2H), 3.21 (s, 3H), 2.90 (d, J=14.6 Hz, 1H), 2.87-2.79 (m, 1H), 2.72 (td, J=15.5, 14.6, 3.1 Hz, 2H), 2.46 (s, 1H), 2.43-2.26 (m, 6H), 2.11-1.99 (m, 1H), 1.82-1.66 (m, 2H), 1.56-1.37 (m, 1H), 0.89 (dt, J=12.3, 7.7 Hz, 12H), 0.35 (s, 3H). LCMS (ESI): m/z: [M+H] calc'd for C₅₁H₆₈N₈O₈ 921.52; found 921.5.

### Example A607. The synthesis of (2R)-2-(((1-(4-(dimethylamino)-4-methylpent-2-ynoyl)azetidin-3-yl)oxy)methyl)-N-((2³S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-piperidinacycloundecaphane-4-yl)-3-methylbutanamide

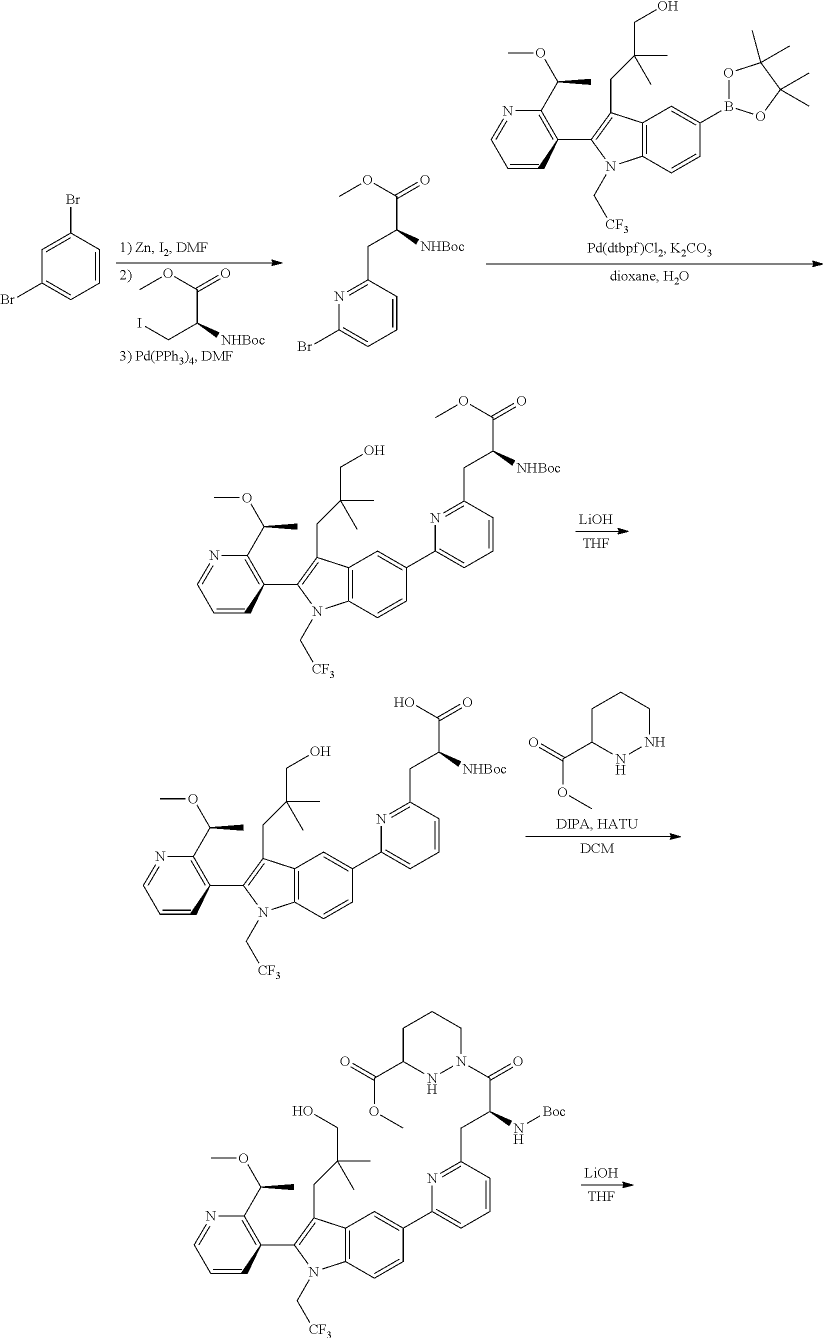

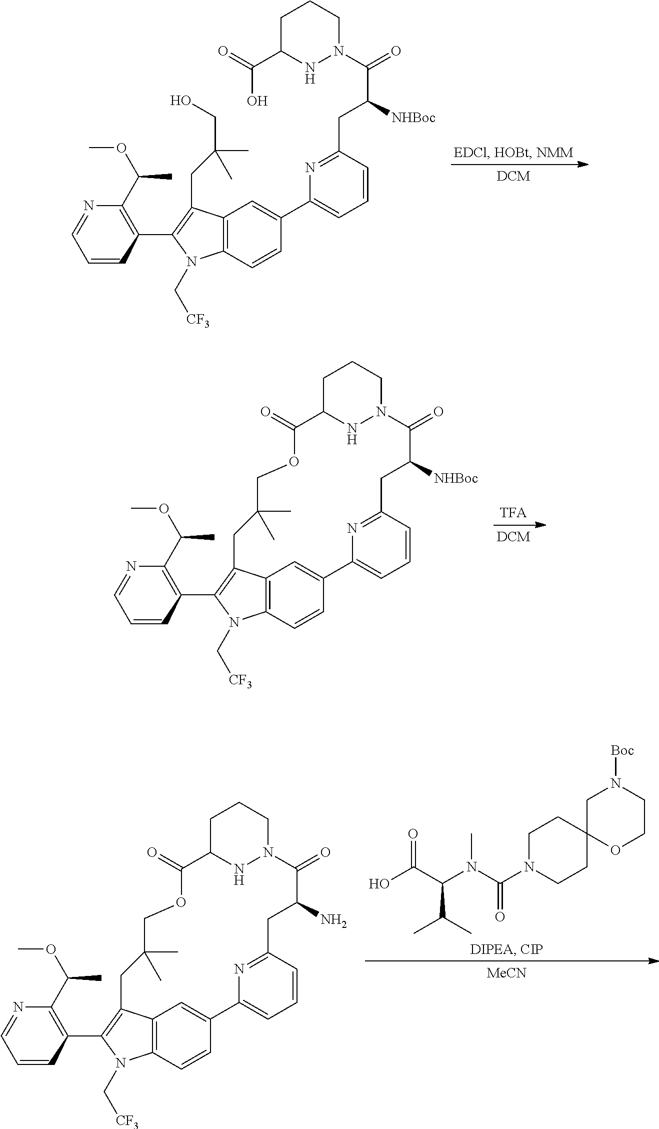

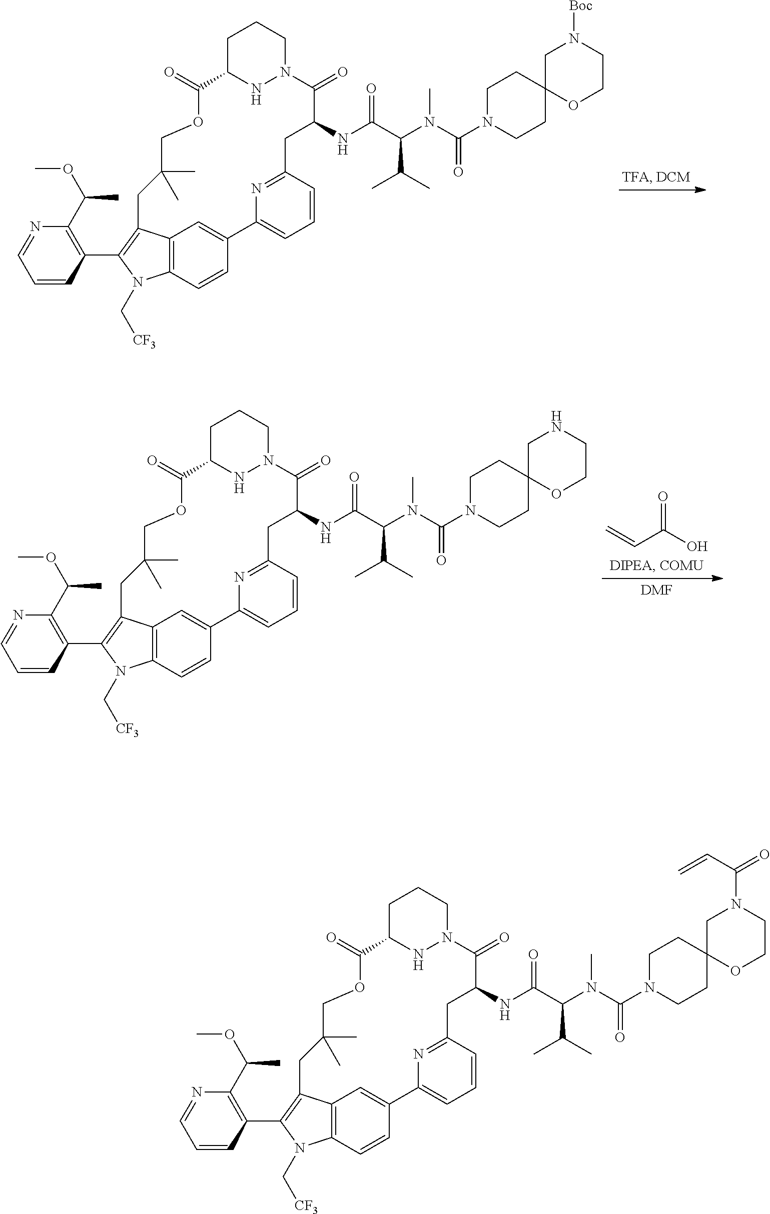

Step 1. A mixture of Zn (44.18 g, 675.41 mmol) and 12 (8.58 g, 33.77 mmol) in DMF (120 mL) was stirred for 30 min at 50° C. under an argon atmosphere, followed by the addition of methyl (2R)-2-[(tert-butoxycarbonyl) amino]-3-iodopropanoate (72.24 g, 219.51 mmol) in DMF (200 mL). The reaction mixture was stirred at 50° C. for 2 h under an argon atmosphere. Then a mixture of 2,6-dibromo-pyridine (40 g, 168.85 mmol) and Pd(PPh₃)₄ (39.02 g, 33.77 mmol) in DMF (200 mL) was added. The resulting mixture was stirred at 75° C. for 2 h, then cooled down to room temperature and extracted with EtOAc (1 L×3). The combined organic layers were washed with H₂O (1 L×3), dried over anhydrous sodium sulfate, filtered and concentrated under reduced pressure. The residue was purified by silica gel column chromatography to afford methyl (2S)-3-(6-bromopyridin-2-yl)-2-[(tert-butoxycarbonyl) amino] propanoate (41 g, 67% yield) as oil. LCMS (ESI): m/z: [M+H] calc'd for C₁₄H₁₉BrN₂O₄ 358.1; found 359.1.

Step 2. To a solution of 3-[(2M)-2-[2-[(1S)-1-methoxyethyl] pyridin-3-yl]-5-(4,4,5,5-tetramethyl-1,3,2-dioxaborolan-2-yl)-1-(2,2,2-trifluoroethyl) indol-3-yl]-2,2-dimethylpropan-1-ol (45.0 g, 82.35 mmol) in dioxane (400 mL) and H₂O (80 mL), were added potassium carbonate (28.45 g, 205.88 mmol), methyl (2S)-3-(6-bromopyridin-2-yl)-2-[(tert-butoxycarbonyl) amino] propanoate (35.5 g, 98.8 mmol), Pd(dtbpf)Cl₂ (5.37 g, 8.24 mmol) at room temperature. The reaction mixture was stirred at 70° C. for 2 h under a nitrogen atmosphere. The resulting mixture was extracted with EtOAc (500 mL×3). The combined organic layers were washed with H₂O (300 mL×3), dried over anhydrous sodium sulfate, filtered and concentrated under reduced pressure. The residue was purified by silica gel chromatography to afford methyl (S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoate (48 g, 83% yield) as solid. LCMS (ESI): m/z: [M+H] calc'd for C₃₇H₄₅F₃N₄O₆ 698.3; found 699.4.

Step 3. A solution of methyl (S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoate (52 g, 74.42 mmol) in THF (520 mL), was added LiOH (74.41 mL, 223.23 mmol) at 0° C. The reaction mixture was stirred at room temperature for 3 h. The resulting mixture was acidified to pH 5 with HCl (aq.) and extracted with EtOAc (1 L×3). The combined organic layers were washed with H₂O (1 L×3), dried over anhydrous sodium sulfate, filtered and concentrated under reduced pressure to afford (S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoic acid (50 g, 98% yield) as solid. LCMS (ESI): m/z: [M+H] calc'd for C₃₆H₄₃F₃N₄O₆ 684.3; found 685.1.

Step 4. To a solution of (S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoic acid (55 g, 80.32 mmol) in DCM (600 mL), were added DIPEA (415.23 g, 3212.82 mmol), and HATU (45.81 g, 120.48 mmol) at 0° C. The reaction mixture was stirred at room temperature for 12 h and then quenched with H₂O. The resulting mixture was extracted with EtOAc (1 L×3). The combined organic layers were washed with H₂O (1 L), dried over anhydrous sodium sulfate, filtered and concentrated under reduced pressure. The residue was purified by silica gel column chromatography to afford methyl 1-((S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoyl)hexahydropyridazine-3-carboxylate (63 g, 96% yield) as solid. LCMS (ESI): m/z: [M+H] calc'd for C₄₂H₅₁F₃N₆O₇ 810.4; found 811.3.

Step 5. A solution of methyl 1-((S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoyl)hexahydropyridazine-3-carboxylate (50 g, 61.66 mmol) in THF (500 mL) and 3M LiOH (61.66 mL, 184.980 mmol) at 0° C. was stirred at room temperature for 3 h, then acidified to pH 5 with HCl (aq.). The resulting mixture was extracted with EtOAc (800 mL×3). The combined organic layers were washed with H₂O (800 mL), dried over anhydrous sodium sulfate, filtered and concentrated under reduced pressure to afford 1-((S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoyl)hexahydropyridazine-3-carboxylic acid (48 g, 97% yield) as solid. LCMS (ESI): m/z: [M+H] calc'd for C₄₁H₅₁F₃N₆O₇ 796.3; found 797.1.

Step 6. To a solution of 1-((S)-2-((tert-butoxycarbonyl)amino)-3-(6-(3-(3-hydroxy-2,2-dimethylpropyl)-2-(2-((S)-1-methoxyethyl)pyridin-3-yl)-1-(2,2,2-trifluoroethyl)-1H-indol-5-yl)pyridin-2-yl)propanoyl)hexahydropyridazine-3-carboxylic acid (50 g, 62.74 mmol) in DCM (10 L) at 0° C., were added DIPEA (243.28 g, 1882.32 mmol), EDCI (360.84 g, 1882.32 mmol) and HOBT (84.78 g, 627.44 mmol). The reaction mixture was stirred at room temperature for 3 h, quenched with H₂O and concentrated under reduced pressure. The residue was extracted with EtOAc (2 L×3). The combined organic layers were washed with H₂O (2 L×3), dried over anhydrous sodium sulfate, filtered and concentrated under reduced pressure. The residue was purified by silica gel column chromatography to afford tert-butyl ((4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-4-yl)carbamate (43.6 g, 89% yield) as solid. LCMS (ESI): m/z: [M+H] calc'd for C₄₁H₄₉F₃N₆O₆ 778.3; found 779.3.

Step 7. To a solution of tert-butyl ((4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-4-yl)carbamate (300 mg) in DCM (10 mL), was added TFA (3 mL) at 0° C. The reaction mixture was stirred at room temperature for 1 h. The resulting mixture was diluted with toluene (10 mL) and concentrated under reduced pressure three times to afford (4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-5,7-dione (280 mg, crude) as oil. LCMS (ESI): m/z: [M+H] calc'd for C₃₆H₄₁F₃N₆O₄ 679.2; found 678.3.

Step 8. To a solution of (4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane- 5,7-dione (140 mg, 0.21 mmol) in MeCN (2 mL), were added DIPEA (266.58 mg, 2.06 mmol), N-(4-(tert-butoxycarbonyl)-1-oxa-4,9-diazaspiro[5.5]undecane-9-carbonyl)-N-methyl-L-valine (127.94 mg, 0.31 mmol) and CIP (114.68 mg, 0.41 mmol) at 0° C. The reaction mixture was stirred at room temperature for 1 h. The resulting mixture was concentrated under reduced pressure. The residue was extracted with EtOAc (10 mL×3). The combined organic layers were washed with H₂O (10 mL×3), dried over anhydrous sodium sulfate, filtered and concentrated under reduced pressure. The residue was purified by silica gel chromatography to afford tert-butyl 9-(((2S)-1-(((6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-4-yl) amino)-3-methyl-1-oxobutan-2-yl) (methyl)carbamoyl)-1-oxa-4,9-diazaspiro [5.5] undecane-4-carboxylate (170 mg, 76% yield) as solid. LCMS (ESI): m/z: [M+H] calc'd for C₅₆H₇₄F₃N₉O₉ 1073.5; found 1074.6.

Step 9. To a solution of tert-butyl 9-(((2S)-1-(((6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-4-yl) amino)-3-methyl-1-oxobutan-2-yl) (methyl)carbamoyl)-1-oxa-4,9-diazaspiro [5.5] undecane-4-carboxylate (160 mg, 0.15 mmol) in DCM (5 mL) at 0° C., was dropwise added TFA (1.5 mL). The reaction mixture was stirred at 0° C. for 1 h. The resulting mixture was diluted with toluene (10 mL) and concentrated under reduced pressure three times to afford N-((2S)-1-(((6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-4-yl) amino)-3-methyl-1-oxobutan-2-yl)-N-methyl-1-oxa-4,9-diazaspiro [5.5]undecane-9-carboxamide (150 mg, crude) as oil. LCMS (ESI): m/z: [M+H] calc'd for C₅₁H₆₅F₃N₉O₇ 973.5; found 974.4.

Step 10. To a solution of N-((2S)-1-(((6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(2,6)-pyridinacycloundecaphane-4-yl) amino)-3-methyl-1-oxobutan-2-yl)-N-methyl-1-oxa-4,9-diazaspiro [5.5] undecane-9-carboxamide (150 mg, 0.15 mmol) in DMF (3 mL), were added DIPEA (199.01 mg, 1.54 mmol), acrylic acid (16.64 mg, 0.23 mmol) and COMU (98.39 mg, 0.23 mmol) at 0° C. The reaction mixture was stirred at room temperature for 1 h and concentrated under reduced pressure. The residue was extracted with EtOAc (10 mL×3). The combined organic layers were washed with H₂O (10 mL×3), dried over anhydrous sodium sulfate, filtered and concentrated under reduced pressure. The residue was purified by silica gel chromatography and reverse phase chromatography to afford 4-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1 W-8-oxa-1(5,3)-indola-6(1,3)-pyridazina-2(2,6)-pyridinacycloundecaphane-4-yl)amino)-3-methyl-1-oxobutan-2-yl)-N-methyl-1-oxa-4,9-diazaspiro[5.5]undecane-9-carboxamide (53 mg, 33% yield) as solid. ¹H NMR (400 MHz, DMSO-d₆) δ 8.79 (dd, J=4.8, 1.9 Hz, 2H), 8.09 (d, J=31.4 Hz, 1H), 7.96 (d, J=8.9 Hz, 1H), 7.90-7.72 (m, 3H), 7.64 (t, J=7.8 Hz, 1H), 7.56 (dd, J=7.8, 4.7 Hz, 1H), 7.03 (s, 1H), 6.86 (dd, J=16.6, 10.4 Hz, 1H), 6.20 (d, J=14.0 Hz, 1H), 5.73 (d, J=12.2 Hz, 2H), 5.47 (s, 1H), 5.35 (d, J=11.8 Hz, 1H), 4.68 (s, 1H), 4.28 (d, J=12.5 Hz, 1H), 4.13 (d, J=6.4 Hz, 1H), 3.92 (s, 1H), 3.84-3.64 (m, 6H), 3.59 (d, J=14.0 Hz, 3H), 3.50 (s, 3H), 3.10 (s, 5H), 3.02 (d, J=13.3 Hz, 2H), 2.85 (d, J=12.1 Hz, 3H), 2.08-1.89 (m, 2H), 1.81 (s, 1H), 1.75-1.51 (m, 5H), 1.39 (d, J=6.1 Hz, 4H), 1.24 (s, OH), 0.91-0.66 (m, 10H), 0.53 (s, 3H). LCMS (ESI): m/z: [M+H] calc'd for C₅₄H₆₈F₃N₉O₈ 1027.5; found 1028.1.

### Example A590. The synthesis of (2R)-2-(((1-(4-(dimethylamino)-4-methylpent-2-ynoyl)azetidin-3-yl)oxy)methyl)-((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)-3-methylbutanamide

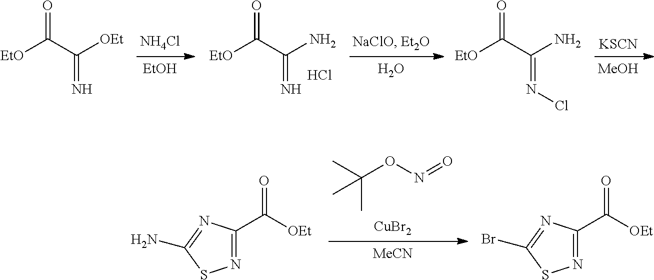

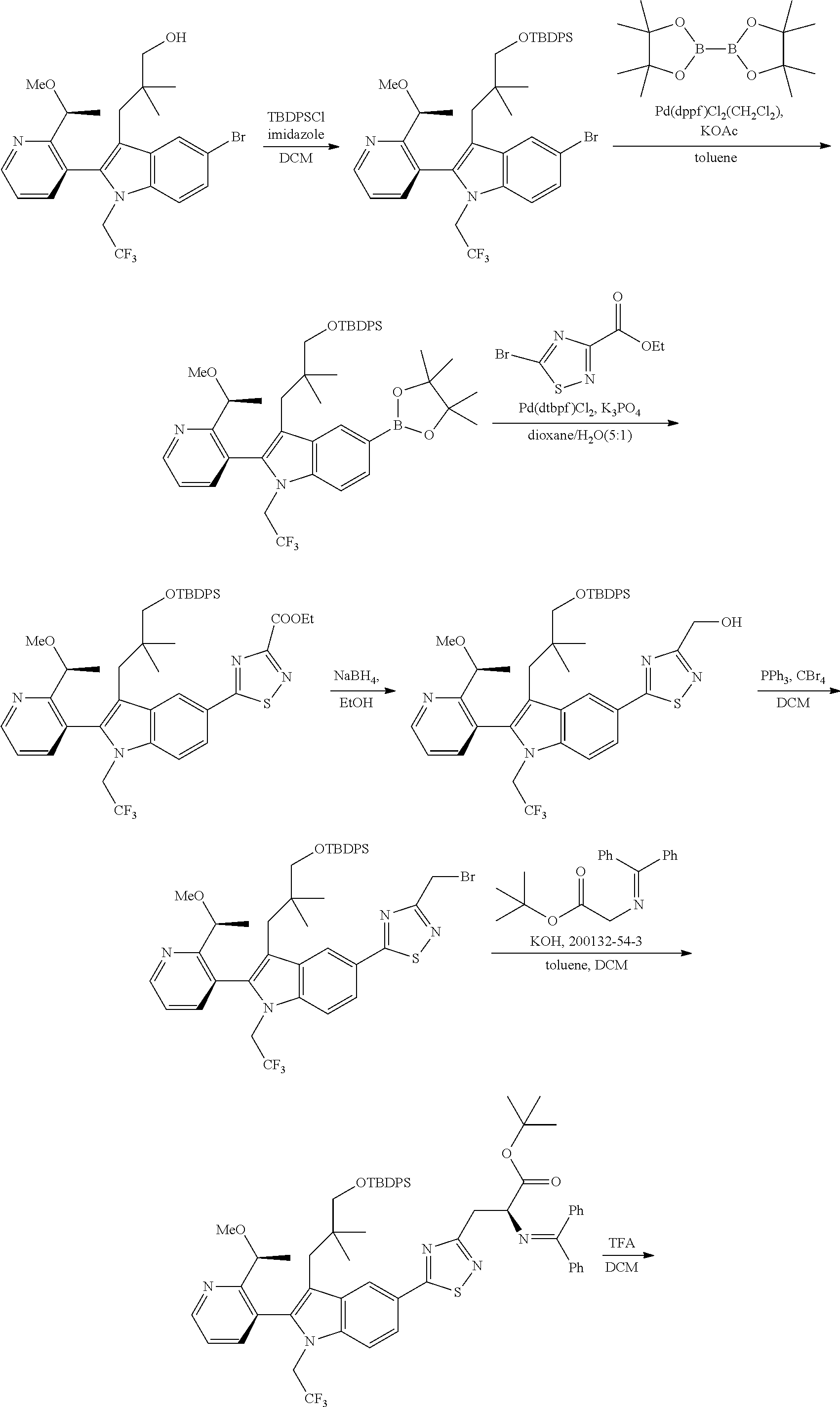

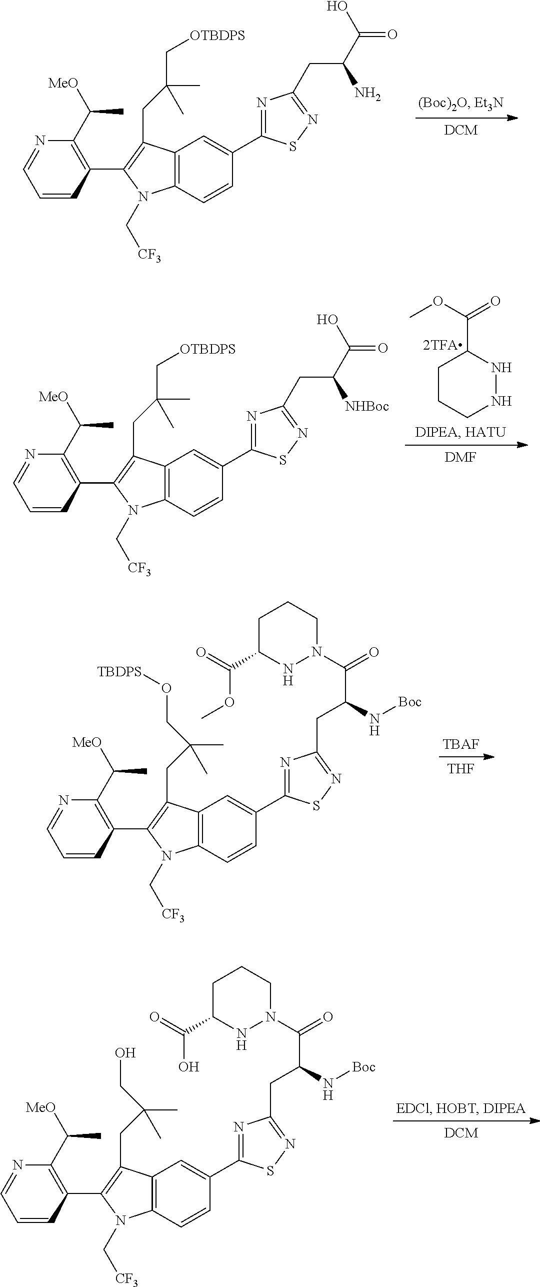

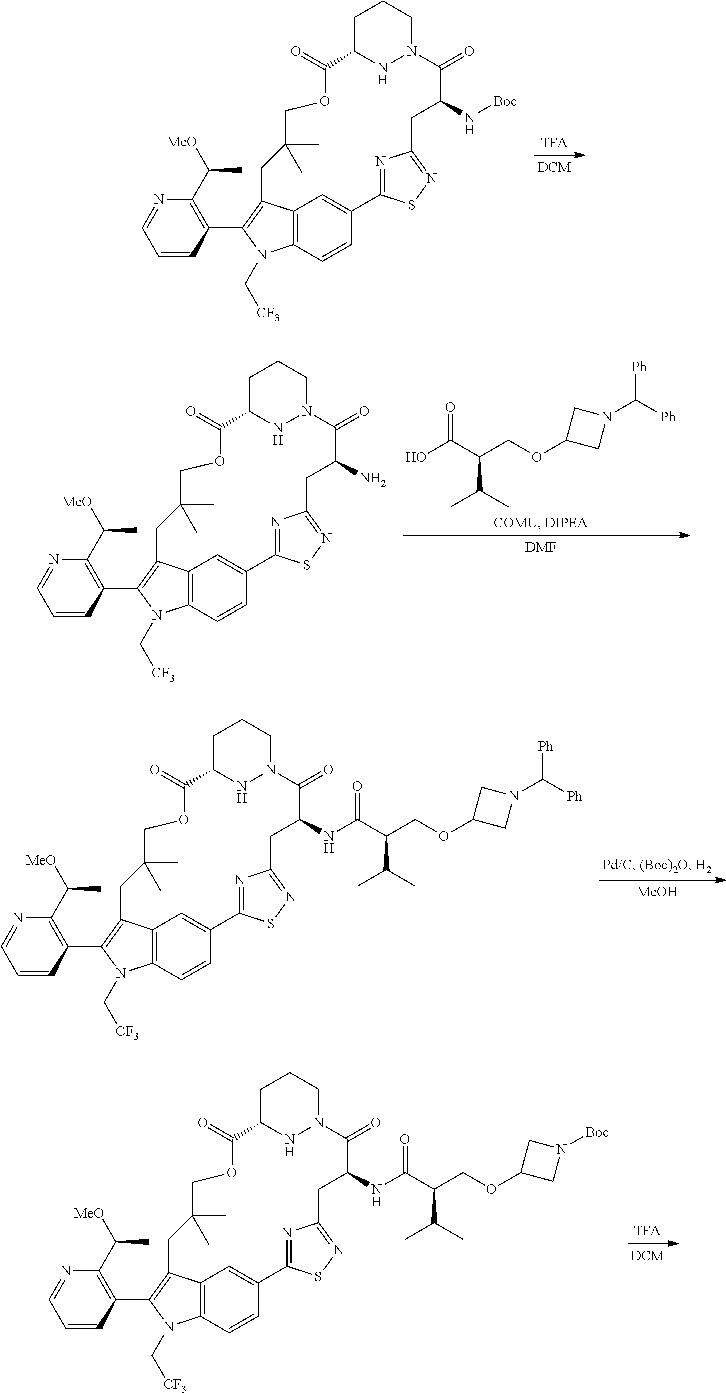

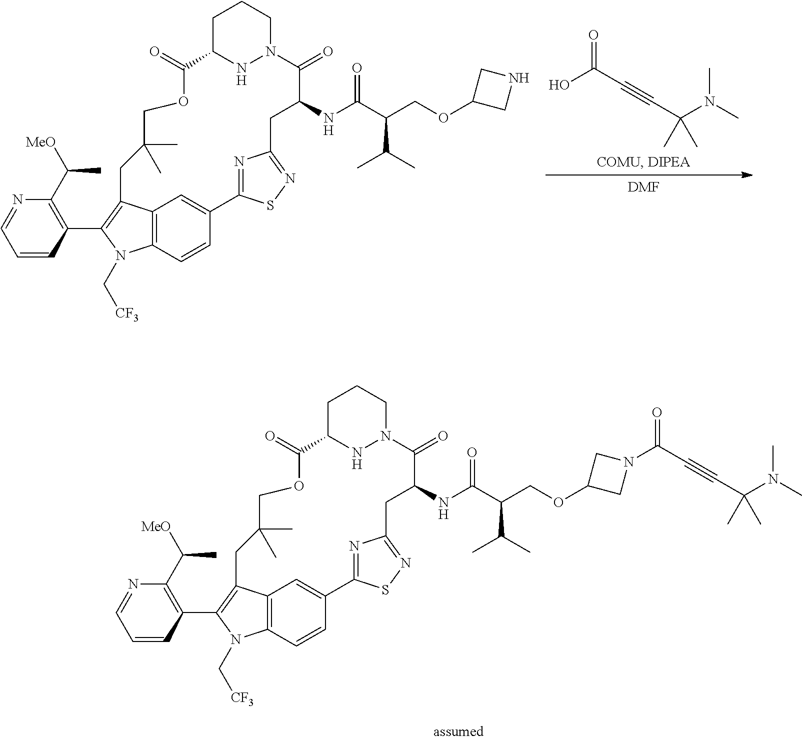

Step 1. To a mixture of ethyl 2-ethoxy-2-iminoacetate (25.0 g, 172.23 mmol) and EtOH (250.0 mL) at 0° C. was added ammonium chloride (9.21 g, 172.23 mmol) in portions then stirred for 4 h at room temperature under an argon atmosphere. The resulting mixture was concentrated under reduced pressure and washed with Et₂O (3×200 mL). The organic layers were combined and concentrated under reduced pressure. This resulted in ethyl 2-amino-2-iminoacetate hydrochloride (20 g, crude) as a light yellow solid. LCMS (ESI): m/z [M+H] calc'd for C₄H₈N₂O₂ 117.07; found 116.9.

Step 2. To a mixture of ethyl 2-amino-2-iminoacetate hydrochloride (13.30 g, 87.17 mmol), H₂O (50.0 mL) and Et₂O (100.0 mL) at 0° C. was added sodium hypochlorite pentahydrate (7,79 g, 104.60 mmol) dropwise. The resulting mixture was stirred for 3 h under an argon atmosphere. The mixture was extracted with Et₂O (3×200 mL). The resulting solution was washed with brine (3×100 mL). The organic phase was dried over anhydrous Na₂SO₄ and concentrated under reduced pressure to afford ethyl (2)-2-amino-2-(chloroimino) acetate (7 g, crude) as a light yellow solid. LCMS (ESI): m/z: [M+H] calc'd for C₄H₇ClN₂O₂ 151.03; found 150.8.

Step 3. To a solution of ethyl (Z)-2-amino-2-(chloroimino) acetate (8.40 g, 55.792 mmol) and MeOH (130.0 mL) at 0° C. was added potassium thiocyanate (5.42 g, 55.79 mmol) in portions. The resulting mixture was stirred for 4 h at room temperature under an argon atmosphere. The reaction was quenched with H₂O/Ice. The mixture was extracted with EtOAc (5×100 mL). The resulting organic phase was dried over anhydrous Na₂SO₄ and concentrated under reduced pressure. The residue was purified by silica gel column chromatography to afford ethyl 5-amino-1, 2, 4-thiadiazole-3-carboxylate (2.3 g, 23.8% yield) as a solid. LCMS (ESI): m/z: [M+H] calc'd for C₅H₇N₃O₂S 174.03; found 173.8.

Step 4. To a solution of ethyl 5-amino-1, 2, 4-thiadiazole-3-carboxylate (5.80 g, 33.49 mmol), MeCN (90.0 mL) and CuBr₂ (11.22 g, 50.23 mmol) at 0° C. was added 2-methyl-2-propylnitrit (6.91 g, 66.98 mmol) dropwise under an argon atmosphere. The mixture was stirred for 30 min. The mixture was then stirred for 4 h at 50° C. The mixture was cooled to 0° C. and quenched with H₂O/Ice. The mixture was extracted with EtOAc (3×100 mL). The resulting organic phase was dried over anhydrous Na₂SO₄ and concentrated under reduced pressure. The residue was purified by silica gel chromatography to afford ethyl 5-bromo-1, 2, 4-thiadiazole-3-carboxylate (6.2 g, 78.1% yield) as a solid. LCMS (ESI): m/z [M+H] calc'd for C₅H₅BrN₂O₂S 236.93; found 237.1.

Step 5. To a solution of (S)-3-(5-bromo-2-(2-(1-methoxyethyl)pyridin-3-yl)-1-(2,2,2-trifluoroethyl)-1H-indol-3-yl)-2,2-dimethylpropan-1-ol (16.60 g, 33.24 mmol), DCM (170.0 mL) and imidazole (5.66 g, 83.10 mmol) at 0° C. was added tert-butyl-chlorodiphenylsilane (11.88 g, 43.21 mmol) dropwise. The resulting mixture was stirred for 3 h at room temperature under an argon atmosphere. The reaction was quenched with H₂O/Ice. The mixture was extracted with EtOAc (3×200 mL). The organic layer was washed with brine (3×100 mL), dried over anhydrous Na₂SO₄ and concentrated under reduced pressure. The residue was purified by silica gel column chromatography to afford (S)-5-bromo-3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indole (22 g, 89.7% yield) as a solid. LCMS (ESI): m/z: [M+H] calc'd for C₃₉H₄₄BrF₃N₂O₂Si 737.24; found 737.0.

Step 6. To a solution of (S)-5-bromo-3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indole (28.0 g, 37.95 mmol), toluene (270.0 mL), KOAc (9.31 g, 94.88 mmol) and bis(pinacolato)diboron (19.27 g, 75.90 mmol) at 0° C. was added Pd(dppf)Cl₂·CH₂Cl₂ (6.18 g, 7.59 mmol) in portions. The resulting mixture was stirred for 3 h at 90° C. under an argon atmosphere. The mixture was cooled to room temperature and quenched with H₂O/Ice. The mixture was extracted with EtOAc (3×200 mL). The resulting organic phase was dried over anhydrous Na₂SO₄ and concentrated under reduced pressure. The residue was purified by silica gel column chromatography to afford (S)-3-(3-((tert-butyldiphenylsilyl)oxy)-2,2-dimethylpropyl)-2-(2-(1-methoxyethyl)pyridin-3-yl)-5-(4,4,5,5-tetramethyl-1,3,2-dioxaborolan-2-yl)-1-(2,2,2-trifluoroethyl)-1H-indole (28.2 g, 94.7% yield) as a solid. LCMS (ESI): m/z: [M+H] calc'd for C₄₅H₅₆BF₃N₂O₄Si 785.41; found 785.4.

Step 7. To a solution of (S)-3-(3-((tert-butyldiphenylsilyl)oxy)-2,2-dimethylpropyl)-2-(2-(1-methoxyethyl)pyridin-3-yl)-5-(4,4,5,5-tetramethyl-1,3,2-dioxaborolan-2-yl)-1-(2,2,2-trifluoroethyl)-1H-indole (19.60 g, 24.97 mmol), 1,4-dioxane (200 mL), H₂O (40 mL), ethyl 5-bromo-1, 2, 4-thiadiazole-3-carboxylate (5.92 g, 24.97 mmol) and K₃PO₄ (13.25 g, 62.43 mmol) at 0° C. was added Pd(dtbpf)Cl₂ (1.63 g, 2.50 mmol) in portions. The resulting mixture was stirred for 1.5 h at 75° C. under an argon atmosphere. The mixture was cooled to 0° C. and quenched with H₂O/Ice and extracted with EtOAc (3×200 mL). The resulting organic phase was dried over anhydrous Na₂SO₄ and concentrated under reduced pressure. The residue was purified by silica gel column chromatography to afford ethyl (S)-5-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-1,2,4-thiadiazole-3-carboxylate (14 g, 68.8% yield) as a yellow solid. LCMS (ESI): m/z: [M+H] calc'd for C₄₄H₄₉F₃N₄O₄SSi 815.33; found 815.2.

Step 8. To a solution of ethyl (S)-5-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-1,2,4-thiadiazole-3-carboxylate (13.60 g, 16.69 mmol) and EtOH (140.0 mL) at 0° C. was added NaBH₄ (3.16 g, 83.43 mmol) in portions. The resulting mixture was stirred for 3 h then quenched with H₂O/Ice. The resulting mixture was washed with brine (3×100 mL) and extracted with EtOAc (3×200 mL). The combined organic layers were dried over anhydrous Na₂SO₄ and concentrated under reduced pressure. The residue was purified by silica gel column chromatography to afford (S)-5-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-1,2,4-thiadiazol-3-ol (9.7 g, 75.2% yield) as a solid. LCMS (ESI): m/z: [M+H] calc'd for C₄₂H₄₇F₃N₄O₃SSi 773.32; found 773.3.

Step 9. To a solution of (S)-5-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-1,2,4-thiadiazol-3-ol (9.70 g, 12.55 mmol), DCM (100.0 mL) and CBr₄ (8.32 g, 25.10 mmol) at 0° C. was added PPh₃ (6.58 g, 25.10 mmol) in DCM (20.0 mL) dropwise. The resulting mixture was stirred for 2 h under an argon atmosphere then quenched with H₂O/Ice. The mixture was extracted with DCM (3×200 mL). The resulting organic phase was dried over anhydrous Na₂SO₄ and concentrated under reduced pressure. The residue was purified by reverse flash chromatography to afford (S)-3-(bromomethyl)-5-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-1,2,4-thiadiazole (9.5 g, 90.6% yield) as a solid. LCMS (ESI): m/z: [M+H] calc'd for C₄₂H₄₆BrF₃N₄O₂SSi 835.23; found 834.9.

Step 10. To a stirred solution of (S)-3-(bromomethyl)-5-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-1,2,4-thiadiazole (9.40 g, 11.25 mmol), toluene (84.0 mL), DCM (36.0 mL), tert-butyl 2-[(diphenylmethylidene)amino]acetate (3.32 g, 11.25 mmol) and O-Allyl-N-(9-anthracenylmethyl)cinchonidinium bromide (0.68 g, 1.13 mmol) at 0° C. was added 9M KOH aqueous (94.0 mL) dropwise. The resulting mixture was stirred overnight under an argon atmosphere. The mixture was extracted with EtOAc (3×200 mL) and the organic phase was concentrated under reduced pressure. The residue was purified by silica gel column chromatography to afford tert-butyl (S)-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)-2-((diphenylmethylene)amino)propanoate (9 g, 76.2% yield) as a solid. LCMS (ESI): m/z: [M+H] calc'd for C₆₁H₆₆F₃NSO₄SSi 1050.46; found 1050.8.

Step 11. To a solution of tert-butyl (S)-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)-2-((diphenylmethylene)amino)propanoate (8.0 g, 7.62 mmol) and DCM (40.0 mL) at 0° C. solution was added TFA (40.0 mL) dropwise. The resulting mixture was stirred overnight at room temperature then concentrated under reduced pressure. The residue was basified to pH 8 with NaHCO₃. The mixture was extracted with EtOAc (3×200 mL). The organic phase was concentrated under reduced pressure. The residue was purified by reverse flash chromatography to afford (S)-2-amino-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoic acid (5 g, 79.1% yield) as a solid. LCMS (ESI): m/z: [M+H] calc'd for C₄₄H₅₀F₃N₅O₄SSi 830.34; found 830.2.

Step 12. To a solution of (S)-2-amino-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoic acid (4.70 g, 5.66 mmol), DCM (50.0 mL) and Et₃N (2.86 g, 28.31 mmol) at 0° C. was added (Boc)₂O (1.36 g, 6.23 mmol) dropwise. The resulting mixture was stirred for 3 h at room temperature under an argon atmosphere then concentrated under reduced pressure and purified by reverse flash chromatography to afford (S)-2-((tert-butoxycarbonyl)amino)-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoic acid (5 g, 94.9% yield) as a solid. LCMS (ESI): m/z: [M+H] calc'd for C₄₉H₅₈F₃N₅O₆SSi 930.39; found 930.3.

Step 13. To a mixture of (S)-2-((tert-butoxycarbonyl)amino)-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoic acid (5.30 g, 5.70 mmol), DMF (60.0 mL), methyl 1,2-diazinane-3-carboxylate (1.64 g, 11.40 mmol) and DIPEA (22.09 g, 170.94 mmol) at 0° C. was added HATU (2.82 g, 7.41 mmol) in DMF (5 mL) dropwise. The resulting mixture was stirred for 3 h at room temperature under an argon atmosphere. The reaction was then quenched with H₂O/Ice. The mixture was extracted with EtOAc (3×100 mL) and the organic phase was washed with brine (3×100 mL). The resulting mixture was dried over anhydrous Na₂SO₄ and concentrated under reduced pressure. The residue was purified by silica gel column chromatography to afford methyl (S)-1-((S)-2-((tert-butoxycarbonyl)amino)-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoyl)hexahydropyridazine-3-carboxylate (5.6 g) as a solid. LCMS (ESI): m/z: [M+H] calc'd for C₅₅H₆₈F₃N₇O₇SSi 1056.47; found 1056.2.

Step 14. A mixture of methyl (S)-1-((S)-2-((tert-butoxycarbonyl)amino)-3-(5-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoyl)hexahydropyridazine-3-carboxylate (5.60 g, 5.30 mmol) and TBAF in THF (56.0 mL) was stirred overnight at 40° C. under an argon atmosphere. The reaction was quenched with sat. NH₄Cl (aq.). The mixture was extracted with EtOAc (3×100 mL) and the organic phase was concentrated under reduced pressure. The residue was purified by reverse flash chromatography to afford (S)-1-((S)-2-((tert-butoxycarbonyl)amino)-3-(5-(3-(3-hydroxy-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoyl)hexahydropyridazine-3-carboxylic acid (4.1 g, 96.2% yield) as a solid. LCMS (ESI): m/z: [M+H] calc'd for C₃₈H₄₈F₃N₇O₇S 804.34; found 804.3.

Step 15. To a solution of (S)-1-((S)-2-((tert-butoxycarbonyl)amino)-3-(5-(3-(3-hydroxy-2,2-dimethylpropyl)-2-(2-((S)-1-methoxyethyl)pyridin-3-yl)-1-(2,2,2-trifluoroethyl)-1H-indol-5-yl)-1,2,4-thiadiazol-3-yl)propanoyl)hexahydropyridazine-3-carboxylic acid (4.0 g, 5.0 mmol) and DCM (450.0 mL) at 0° C. were added DIPEA (51.45 g, 398.08 mmol), HOBt (6.72 g, 49.76 mmol) and EDCI (57.23 g, 298.55 mmol) in portions. The resulting mixture was stirred for 16 h at room temperature under an argon atmosphere. The reaction was quenched with H₂O/Ice and extracted with EtOAc (3×30 mL). The organic phase was concentrated under reduced pressure. The residue was purified by silica gel column chromatography to afford tert-butyl ((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)carbamate (1.7 g, 43.5% yield) as a solid. LCMS (ESI): m/z [M+H] calc'd for C₃₆H₄₆F₃N₇O₆S 786.33; found 786.3.

Step 16. To a solution of ((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)carbamate (300.0 mg, 0.38 mmol) and DCM (2.0 mL) at 0° C. was added TFA (1.0 mL) dropwise. The resulting mixture was stirred for 2 h at room temperature then concentrated under reduced pressure. The residue was basified to pH 8 with saturated NaHCO₃ (aq.). The mixture was extracted with EtOAc (3×20 mL). The organic phase was concentrated under reduced pressure to afford (6³S,4S,Z)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-5,7-dione (270 mg, crude) as a solid. LCMS (ESI): m/z: [M+H] calc'd for C₃₃H₃F₃N₇O₄S 686.27; found 686.1.

Step 17. To a solution of (6³S,4S,Z)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-5,7-dione (160.0 mg, 0.23 mmol), (R)-2-(((1-benzhydrylazetidin-3-yl)oxy)methyl)-3-methylbutanoic acid (123.70 mg, 0.35 mmol) and DMF (2.0 mL) at 0° C. were added DIPEA (603.09 mg, 4.660 mmol) and COMU (119.91 mg, 0.28 mmol) in DMF (0.5 mL). The resulting mixture was stirred for 2 h at room temperature under an argon atmosphere. The reaction was quenched with H₂O/Ice and extracted with EtOAc (3×20 mL). The organic phase was washed with brine (3×10 mL) and concentrated under reduced pressure. The residue was purified by Prep-TLC to afford (2R)-2-(((1-benzhydrylazetidin-3-yl)oxy)methyl)-N-((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)-3-methylbutanamide (160 mg, 67.2% yield) as a solid. LCMS (ESI): m/z: [M+H] calc'd for C₅₅H₆₃F₃N₈O₆S 1021.46; found 1021.4.

Step 18. To a solution of (2R)-2-(((1-benzhydrylazetidin-3-yl)oxy)methyl)-N-((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)-3-methylbutanamide (160.0 mg, 0.16 mmol) and MeOH (5.0 mL) at 0° C. was added (Boc)₂O (85.49 mg, 0.39 mmol) dropwise followed by Pd/C (320.0 mg) in portions. The resulting mixture was stirred overnight at room temperature under a hydrogen atmosphere. The resulting mixture was filtered and the filter cake was washed with EtOAc (3×20 mL). The filtrate was concentrated under reduced pressure. The residue was purified by Prep-TLC to afford tert-butyl 3-((2R)-2-(((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)carbamoyl)-3-methylbutoxy)azetidine-1-carboxylate (80 mg, 53.5% yield) as a solid. LCMS (ESI): m/z: [M+H] calc'd for C₄₇H₆₁F₃N₈O₈S 955.44; found 955.2.

Step 19. To a solution of tert-butyl 3-((2R)-2-(((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)carbamoyl)-3-methylbutoxy)azetidine-1-carboxylate (120.0 mg, 0.13 mmol) and DCM (0.80 mL) at 0° C. was added TFA (0.4 mL) dropwise and the resulting mixture was stirred for 2 h at room temperature. The mixture was basified to pH 8 with saturated NaHCO₃ (aq.). The mixture was extracted with EtOAc (3×10 mL) and concentrated under reduced pressure. The residue was purified by reverse flash chromatography to afford (2R)-2-((azetidin-3-yloxy)methyl)-N-((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)-3-methylbutanamide (40 mg, 37.2% yield) as a solid. LCMS (ESI): m/z [M+H] calc'd for C₄₂H₅₃F₃N₈O₆S 855.38; found 855.3.

Step 20. To a solution of (2R)-2-((azetidin-3-yloxy)methyl)-N-((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)-3-methylbutanamide (32.0 mg, 0.037 mmol),4-(dimethylamino)-4-methylpent-2-ynoic acid (11.62 mg, 0.074 mmol) and DMF (0.50 mL) at 0° C. were added DIPEA (193.49 mg, 1.48 mmol) and COMU (19.23 mg, 0.044 mmol) in DMF (0.1 mL) dropwise. The resulting mixture was stirred for 2 h at room temperature under an argon atmosphere. The reaction was quenched with H₂O/Ice and extracted with EtOAc (3×20 mL). The organic phase was washed with brine (3×10 mL) and concentrated under reduced pressure. The crude product (60 mg) was purified by reverse phase chromatography to afford (2R)-2-(((1-(4-(dimethylamino)-4-methylpent-2-ynoyl)azetidin-3-yl)oxy)methyl)-N-((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(5,3)-thiadiazola-1(5,3)-indola-6(1,3)-pyridazinacycloundecaphane-4-yl)-3-methylbutanamide (11.7 mg, 30.9% yield) as a solid. ¹H NMR (400 MHz, DMSO-d₆) δ 8.80 (dd, J=4.7, 1.8 Hz, 1H), 8.58 (s, 1H), 8.30 (d, J=8.9 Hz, 1H), 7.92 (d, J=8.6 Hz, 1H), 7.84-7.69 (m, 2H), 7.56 (dd, J=7.8, 4.8 Hz, 1H), 5.78 (t, J=8.6 Hz, 2H), 5.10 (d, J=12.1 Hz, 1H), 4.91 (dd, J=16.9, 8.8 Hz, 1H), 4.31 (d, J=6.6 Hz, 6H), 4.05 (dd, J=16.3, 6.6 Hz, 2H), 3.87 (d, J=6.3 Hz, 1H), 3.75 (d, J=11.3 Hz, 1H), 3.61 (d, J=11.0 Hz, 1H), 3.53 (d, J=9.8 Hz, 1H), 3.45 (s, 3H), 3.25 (s, 1H), 3.09 (d, J=10.4 Hz, 1H), 3.01 (d, J=14.5 Hz, 1H), 2.79 (s, 1H), 2.45-2.35 (m, 2H), 2.20 (d, J=5.4 Hz, 6H), 2.15 (d, J=12.0 Hz, 1H), 1.81 (d, 2H), 1.74-1.65 (m, 1H), 1.54 (s, 1H), 1.41-1.30 (m, 9H), 1.24 (s, 1H), 0.94 (s, 3H), 0.90-0.81 (m, 5H), 0.29 (s, 3H). LCMS (ESI): m/z: [M+H] calc'd for C₅₀H₆₄F₃N₉O₇S 992.47; found 992.5.

The following table of compounds (Table 3) were prepared using the aforementioned methods or variations thereof, as is known to those of skill in the art.

TABLE 3
Exemplary Compounds Prepared by Methods of the Present Invention

| | LCMS (ESI): m/z | | LCMS (ESI): m/z |
|---|---|---|---|
| Ex# | [M + H] Found | Ex# | [M + H] Found |
| A1 | 944.5 | A116 | 986.5 |
| A2 | 888.6 | A117 | 879.5 |
| A3 | 903.2 | A118 | 881.6 |
| A4 | 876.3 | A119 | 874.5 |
| A5 | 903.4 | A120 | 929.7 |
| A6 | 945.8 | A121 | 943.6 |
| A7 | 1058.1 | A122 | 888.8 |
| A8 | 960.8 | A123 | 959.3 |
| A9 | 921.7 | A124 | 924.6 |
| A10 | 928.8 | A125 | 962.5 |
| A11 | 936.4 | A126 | 986.5 |
| A12 | 942.5 | A127 | 946.6 |
| A13 | 932.5 | A128 | 903.4 |
| A14 | 917.5 | A129 | 1001.2 |
| A15 | 961.5 | A130 | 930.5 |
| A16 | 890.6 | A131 | 959.5 |
| A17 | 875.4 | A132 | 940.5 |
| A18 | 918.5 | A133 | 969.7 |
| A19 | 975.6 | A134 | 990.5 |
| A20 | 934.5 | A135 | 933.5 |
| A21 | 903.3 | A136 | 875.4 |
| A22 | 903.6 | A137 | 889.5 |
| A23 | 844.6 | A138 | 929.4 |
| A24 | 887.6 | A139 | 930.4 |
| A25 | 921.4 | A140 | 943.7 |
| A26 | 930.4 | A141 | 943.8 |
| A27 | 915.6 | A142 | 901.7 |
| A28 | 890.6 | A143 | 852.3 |
| A29 | 903.5 | A144 | 886.7 |
| A30 | 902.7 | A145 | 916.7 |
| A31 | 901.4 | A146 | 963.4 |
| A32 | 1003.4 | A147 | 864.4 |
| A33 | 917.4 | A148 | 885.5 |
| A34 | 942.7 | A149 | 947.5 |
| A35 | 956.1 | A150 | 902.7 |
| A36 | 1005.5 | A151 | 904.5 |
| A37 | 917.5 | A152 | 885.5 |
| A38 | 959.5 | A153 | 949.6 |
| A39 | 942.9 | A154 | 907.5 |
| A40 | 915.7 | A155 | 944.5 |
| A41 | 917.5 | A156 | 943.7 |
| A42 | 888.6 | A157 | 943.5 |
| A43 | 942.5 | A158 | 904.5 |
| A44 | 904.35 | A159 | 904.5 |
| A45 | 846.35 | A160 | 858.5 |
| A46 | 982.5 | A161 | 947.4 |
| A47 | 960.1 | A162 | 973.5 |
| A48 | 905.3 | A163 | 989.4 |
| A49 | 981.8 | A164 | 947.5 |
| A50 | 916.9 | A165 | 960.3 |
| A51 | 946.7 | A166 | 907.7 |
| A52 | 916.5 | A167 | 918.5 |
| A53 | 921.6 | A168 | 907.4 |
| A54 | 947.7 | A169 | 859.4 |
| A55 | 916.5 | A170 | 874.3 |
| A56 | 904.5 | A171 | 867.5 |
| A57 | 930.5 | A172 | 883.6 |
| A58 | 895.5 | A173 | 900.5 |
| A59 | 944.7 | A174 | 848.4 |
| A60 | 931.5 | A175 | 959.5 |
| A61 | 847.7 | A176 | 944.5 |
| A62 | 871.5 | A177 | 935.4 |
| A63 | 930.4 | A178 | 930.4 |
| A64 | 893.7 | A179 | 849.5 |
| A65 | 874.7 | A180 | 849.5 |
| A66 | 876.5 | A181 | 918.8 |
| A67 | 970.5 | A182 | 1002.2 |
| A68 | 91 5.4 | A183 | 961.5 |
| A69 | 927.3 | A184 | 1003.5 |
| A70 | 876.4 | A185 | 928.5 |
| A71 | 933.5 | A186 | 992.5 |
| A72 | 1003.5 | A187 | 945.5 |
| A73 | 903.6 | A188 | 960.4 |
| A74 | 885.5 | A18S | 1000.6 |
| A75 | 904.7 | A190 | 934.3 |
| A76 | 904.5 | A191 | 985.5 |
| A77 | 860.6 | A192 | 971.5 |
| A78 | 1018.5 | A193 | 871.6 |
| A79 | 947.5 | A194 | 918.5 |
| A80 | 933.3 | A195 | 927.4 |
| A81 | 917.8 | A196 | 972.3 |
| A82 | 908.7 | A197 | 896.4 |
| A83 | 890.6 | A198 | 916.6 |
| A84 | 890.6 | A199 | 916.6 |
| A85 | 891.5 | A200 | 987.4 |
| A86 | 885.5 | A201 | 993.7 |
| A87 | 947.7 | A202 | 896.4 |
| A88 | 945.5 | A203 | 987.6 |
| A89 | 944.8 | A204 | 987.6 |
| A90 | 898 | A205 | 933.5 |
| A91 | 890.5 | A206 | 930.5 |
| A92 | 903.4 | A207 | 930.5 |
| A93 | 901.4 | A208 | 958.8 |
| A94 | 881.5 | A20S | 1015.7 |
| A95 | 987.8 | A210 | 906.5 |
| A96 | 945.7 | A211 | 905.5 |
| A97 | 931.8 | A212 | 901.3 |
| ASS | 943.8 | A213 | 969.5 |
| A99 | 849.7 | A214 | 933.6 |
| A100 | 942.8 | A215 | 890.3 |
| A101 | 893.4 | A216 | 916.4 |
| A102 | 904.4 | A217 | 930.5 |
| A103 | 947.5 | A218 | 983.5 |
| A104 | 931.7 | A219 | 917.4 |
| A105 | 915.6 | A220 | 996.5 |
| A106 | 902.5 | A221 | 915.4 |
| A107 | 945.7 | A222 | 930.05 |
| A108 | 902.3 | A223 | 920.01 |
| A109 | 906.5 | A224 | 983.5 |
| A110 | 793.4 | A225 | 982.7 |
| A111 | 946.7 | A226 | 873.7 |
| A112 | 905.14 | A227 | 887.7 |
| A113 | 849.6 | A228 | 911 |
| A114 | 888.4 | A229 | 896.7 |
| A115 | 888.4 | | |
| A230 | 905.5 | A286 | 877.4 |
| A231 | 912.4 | A287 | 894.7 |
| A232 | 849.4 | A288 | 879.3 |
| A233 | 905.4 | A289 | 908.7 |
| A234 | 1053.3 | A290 | 971.6 |
| A235 | 835.5 | A291 | 978.5 |
| A236 | 882.3 | A292 | 987.5 |
| A237 | 1015.4 | A293 | 921.4 |
| A238 | 971.5 | A294 | 902.8 |
| A239 | 869.4 | A295 | 916.4 |
| A240 | 884.4 | A296 | 930.8 |
| A241 | 987.4 | A297 | 938.4 |
| A242 | 877.4 | A298 | 952.6 |
| A243 | 863.5 | A299 | 966.7 |
| A244 | 1029.6 | A300 | 919.6 |
| A245 | 1029.5 | A301 | 907.7 |
| A246 | 501.4 | A302 | 895.8 |
| A247 | 985.5 | A303 | 895.4 |
| A248 | 896.4 | A304 | 897.5 |
| A249 | 984.5 | A305 | 968.6 |
| A250 | 927.4 | A306 | 968.7 |
| A251 | 985.4 | A307 | 952.6 |
| A252 | 927.3 | A308 | 952.7 |
| A253 | 990.4 | A309 | 933.6 |
| A254 | 933.3 | A310 | 926.8 |
| A255 | 930.7 | A311 | 944.7 |
| A256 | 890.4 | A312 | 897.5 |
| A257 | 947.4 | A313 | 976.5 |
| A258 | 990.3 | A314 | 893.3 |
| A259 | 933.3 | A316 | 930.5 |
| A260 | 918.4 | A317 | 982.6 |
| A261 | 975.5 | A318 | 883.5 |
| A262 | 919.4 | A319 | 916.5 |
| A263 | 976.5 | A320 | 886.6 |
| A264 | 990.5 | A321 | 900.6 |
| A265 | 933.5 | A322 | 1004.4 |
| A266 | 945.5 | A323 | 976.5 |
| A267 | 987.5 | A324 | 969.5 |
| A268 | 973.5 | A325 | 1033.7 |
| A269 | 487.3 | A326 | 997.5 |
| A270 | 959.4 | A327 | 906.6 |
| A271 | 861.3 | A328 | 986.7 |
| A272 | 847.6 | A329 | 890.5 |
| A273 | 861.6 | A330 | 895.6 |
| A274 | 930.5 | A331 | 879.5 |
| A275 | 916.3 | A332 | 942.5 |
| A276 | 892.8 | A333 | 919.6 |
| A277 | 895.7 | A334 | 930.7 |
| A278 | 879.5 | A335 | 924.5 |
| A279 | 895.4 | A336 | 927.4 |
| A280 | 954.6 | A337 | 501.4 |
| A281 | 916.6 | A338 | 919.4 |
| A282 | 952.5 | A339 | 971.6 |
| A283 | 931.4 | A340 | 895.7 |
| A284 | 889.5 | A341 | 938.5 |
| A285 | 839.4 | A342 | 906.8 |
| A343 | 992.4 | A370 | 1087.3 |
| A344 | 901.40 | A371 | 902.5 |
| A345 | 897.40 | A372 | 992.1 |
| A346 | 942.50 | A373 | 902.6 |
| A347 | 896.30 | A374 | 1,034.40 |
| A348 | 1,002.30 | A375 | 993.50 |
| A349 | 924.6 | A376 | 984.40 |
| A350 | 968.2 | A377 | 939.50 |
| A351 | 997.6 | A378 | 897.50 |
| A352 | 952.7 | A379 | 1005.6 |
| A353 | 966.3 | A380 | 949.4 |
| A354 | 966.2 | A381 | 921.5 |
| A355 | 855.6 | A382 | 938.5 |
| A356 | 910.4 | A383 | 938.5 |
| A357 | 1091.0 | A384 | 924.5 |
| A358 | 902.5 | A385 | 938.3 |
| A359 | 904.7 | A386 | 938.4 |
| A360 | 924.5 | A387 | 938.5 |
| A361 | 967.5 | A388 | 938.5 |
| A362 | 936.6 | A389 | 936.5 |
| A363 | 961.4 | A390 | 924.5 |
| A364 | 946.5 | A391 | 1011.6 |
| A365 | 995.6 | A392 | 958.0 |
| A366 | 921.5 | A393 | 922.4 |
| A367 | 950.6 | A394 | 950.3 |
| A368 | 924.5 | A395 | 936.5 |
| A369 | 924.5 | A396 | 924.4 |
| A397 | 924.7 | A424 | 934.6 |
| A398 | 914.6 | A425 | 1012.5 |
| A399 | 902.5 | A426 | 912.5 |
| A400 | 956.6 | A427 | 895.30 |
| A401 | 95b. 9 | A428 | 951.30 |
| A402 | 940.8 | A429 | 911.50 |
| A403 | 948.40 | A430 | 938.5 |
| A404 | 980.40 | A431 | 952.3 |
| A405 | 911.50 | A432 | 924.2 |
| A406 | 938.7 | A433 | 924.2 |
| A407 | 922.5 | A434 | 924.7 |
| A408 | 922.5 | A435 | 910.5 |
| A409 | 922.6 | A436 | 924.6 |
| A410 | 1046.9 | A437 | 912.5 |
| A411 | 990.7 | A438 | 898.5 |
| A412 | 912.7 | A439 | 1016.6 |
| A413 | 914.7 | A440 | 990.5 |
| A414 | 983.5 | A441 | 939.6 |
| A415 | 950.5 | A442 | 889.2 |
| A416 | 924.6 | A443 | 913.5 |
| A417 | 936.7 | A444 | 913.5 |
| A418 | 938.5 | A445 | 890.1 |
| A419 | 922.3 | A446 | 957.6 |
| A420 | 936.7 | A447 | 880.5 |
| A421 | 924.5 | A448 | 888.4 |
| A422 | 910.5 | A449 | 920.5 |
| A423 | 1028.9 | A450 | 897.2 |
| A451 | 902.6 | A478 | 900.5 |
| A452 | 888.4 | A479 | 993.6 |
| A453 | 901.3 | A480 | 902.2 |
| A454 | 1044.6 | A481 | 930.6 |
| A455 | 949.2 | A482 | 974.6 |
| A456 | 1059.5 | A483 | 847.5 |
| A457 | 916.7 | A484 | 847.4 |
| A458 | 916.2 | A485 | 938.4 |
| A459 | 902.3 | A486 | 893.5 |
| A460 | 874.5 | A487 | 900.6 |
| A461 | 957.9 | A488 | 888.5 |
| A462 | 854.3 | A489 | 962.6 |
| A463 | 910.7 | A490 | 896.5 |
| A464 | 964.5 | A491 | 910.2 |
| A465 | 964.5 | A492 | 910.5 |
| A466 | 1033.7 | A493 | 910.5 |
| A467 | 874.5 | A494 | 886.3 |
| A468 | 879.6 | A495 | 907.5 |
| A469 | 902.6 | A496 | 900.4 |
| A470 | 900.6 | A497 | 902.7 |
| A471 | 900.6 | A498 | 902.6 |
| A472 | 964.6 | A499 | 998.7 |
| A473 | 854.3 | A500 | 897.5 |
| A474 | 914.4 | A501 | 906.3 |
| A475 | 914.40 | A502 | 949.2 |
| A476 | 983.6 | A503 | 920.6 |
| A477 | 907.5 | A504 | 920.8 |
| A505 | 920.0 | A532 | 1013.6 |
| A506 | 879.3 | A533 | 999.3 |
| A507 | 897.4 | A534 | 983.4 |
| A508 | 942.4 | A535 | 919.5 |
| A509 | 942.4 | A536 | 961.7 |
| A510 | 899.3 | A537 | 961.6 |
| A511 | 919.5 | A532 | 1013.6 |
| A512 | 900.5 | A533 | 999.3 |
| A513 | 983.7 | A534 | 983.4 |
| A514 | 983.7 | A535 | 919.5 |
| A515 | 985.7 | A536 | 961.7 |
| A516 | 985.7 | A537 | 961.6 |
| A517 | 892.6 | A538 | 999.7 |
| A518 | 920.6 | A539 | 960.4 |
| A519 | 920.4 | A540 | 1050.2 |
| A520 | 990.6 | A541 | 1078.7 |
| A521 | 990.6 | A542 | 1155.7 |
| A522 | 945.3 | A543 | 952.6 |
| A523 | 945.4 | A544 | 952.6 |
| A524 | 914.5 | A545 | 981.2 |
| A525 | 914.4 | A546 | 922.3 |
| A526 | 1005.4 | A547 | 922.5 |
| A527 | 972.4 | A548 | 950.5 |
| A528 | 985.4 | A549 | 997.4 |
| A529 | 987.4 | A550 | 1025.6 |
| A530 | 898.5 | A551 | 997.5 |
| A531 | 912.3 | A552 | 950.4 |
| A553 | 974.6 | A580 | 966.6 |
| A554 | 957.5 | A581 | 966.6 |
| A555 | 1054.4 | A582 | 953.5 |
| A556 | 972.5 | A583 | 938.5 |
| A557 | 952.4 | A584 | 944.7 |
| A558 | 959.6 | A585 | 944.6 |
| A559 | 1011.7 | A586 | 950.6 |
| A560 | 1011.6 | A587 | 962.6 |
| A561 | 922.5 | A588 | 938.5 |
| A562 | 954.3 | A589 | 1016.6 |
| A563 | 978.5 | A590 | 992.5 |
| A564 | 982.5 | A591 | 1008.7 |
| A565 | 1039.3 | A592 | 997.5 |
| A566 | 1039.4 | A593 | 1020.5 |
| A567 | 1039.4 | A594 | 1020.6 |
| A568 | 1039.3 | A595 | 1000.6 |
| A569 | 997.4 | A596 | 966.7 |
| A570 | 974.7 | A597 | 946.6 |
| A571 | 1038.5 | A598 | 979.6 |
| A572 | 1056.5 | A599 | 958.7 |
| A573 | 1063.5 | A600 | 887.4 |
| A574 | 990.6 | A601 | 985.5 |
| A575 | 1004.5 | A602 | 1036.5 |
| A576 | 991.5 | A603 | 989.9 |
| A577 | 1062.3 | A604 | 1006.1 |
| A578 | 1048.3 | A605 | 1059.7 |
| A579 | 963.3 | A606 | 1032.6 |
| A607 | 1028.1 | A634 | 938.6 |
| A608 | 1027.1 | A635 | 978.6 |
| A609 | 966.6 | A636 | 916.6 |
| A610 | 945.6 | A637 | 909.5 |
| A611 | 1021.5 | A638 | 909.5 |
| A612 | 920.6 | A639 | 928.3 |
| A613 | 956.1 | A640 | 942.0 |
| A614 | 992.5 | A641 | 942.6 |
| A615 | 1010.5 | A642 | 1047.5 |
| A616 | 1010.5 | A643 | 966.6 |
| A617 | 990.5 | A644 | 944.4 |
| A618 | 950.7 | A645 | 986.5 |
| A619 | 950.4 | A646 | 937.5 |
| A620 | 938.1 | A647 | 1012.5 |
| A621 | 966.1 | A648 | 944.5 |
| A622 | 982.6 | A649 | 921.6 |
| A623 | 999.6 | A650 | 1007.6 |
| A624 | 1019.5 | A651 | 964.2 |
| A625 | 1019.5 | A652 | 1903.1 |
| A626 | 913.5 | A653 | 938.6 |
| A627 | 913.4 | A654 | 938.6 |
| A628 | 1046.5 | A655 | 934.4 |
| A629 | 952.2 | A656 | 1005.5 |
| A630 | 967.6 | A657 | 960.7 |
| A631 | 942.5 | A658 | 1003.3 |
| A632 | 942.6 | A659 | 974.6 |
| A633 | 938.6 | A660 | 925.5 |
| A661 | 936.6 | A688 | 936.5 |
| A662 | 936.6 | A689 | 1036.9 |
| A663 | 935.5 | A690 | 979.6 |
| A664 | 1047.5 | A691 | 923.5 |
| A665 | 1035.5 | A692 | 962.5 |
| A666 | 1035.2 | A693 | 906.5 |
| A667 | 936.3 | A694 | 896.1 |
| A668 | 965.6 | A695 | 980.7 |
| A669 | 1049.7 | A696 | 978.4 |
| A670 | 1047.7 | A697 | 978.3 |
| A671 | 964.6 | A698 | 964.3 |
| A672 | 997.9 | A699 | 1003.3 |
| A673 | 1015.3 | A700 | 927.2 |
| A674 | 975.5 | A701 | 993.7 |
| A675 | 921.5 | A702 | 950.6 |
| A676 | 966.5 | A703 | 985.5 |
| A677 | 966.4 | A704 | 938.6 |
| A678 | 950.5 | A705 | 925.5 |
| A679 | 990.2 | A706 | 952.5 |
| A680 | 943.6 | A707 | 1016.5 |
| A681 | 980.6 | A708 | 903.2 |
| A682 | 968.6 | A709 | 981.5 |
| A683 | 1000.6 | A710 | 967.5 |
| A684 | 915.0 | A711 | 964.5 |
| A685 | 977.6 | A712 | 937.9 |
| A686 | 922.5 | A713 | 939.9 |
| A687 | 936.4 | A714 | 952.5 |
| A715 | 937.4 | A733 | 997.6 |
| A716 | 893.5 | A734 | 936.5 |
| A717 | 924.6 | A735 | 952.5 |
| A718 | 936.6 | A736 | 922.2 |
| A719 | 950.5 | A737 | 922.1 |
| A720 | 935.3 | A738 | 924.5 |
| A721 | 937.3 | A739 | 924.5 |
| A722 | 929.4 | A730 | 993.2 |
| A723 | 910.4 | A731 | 922.7 |
| A724 | 985.4 | A732 | 928.6 |
| A725 | 919.3 | A733 | 997.6 |
| A726 | 1010.4 | A734 | 936.5 |
| A727 | 1025.5 | A735 | 952.5 |
| A728 | 924.6 | A736 | 922.2 |
| A729 | 993.5 | A737 | 922.1 |
| A730 | 993.2 | A738 | 924.5 |
| A731 | 922.7 | A739 | 924.5 |
| A732 | 928.6 | A740 | 895.5 |
| A741 | 895.4 | | |

Blank = not determined

### Matched Pair Analysis

FIGS. 1A-1B compare the potency in two different cell-based assays of compounds of Formula BB of the present invention (points on the right) and corresponding compounds of Formula AA (points on the left) wherein a H is replaced with (S)Me. The y axes represent pERK EC50 (FIG. 1A) or CTG IC50 (FIG. 1B) as measured in an H358 cell line. Assay protocols are below. The linked points represent a matched pair that differs only between H and (S)Me substitution. Each compound of Formula BB demonstrated reduced potency in cell assays compared to the corresponding compound of Formula AA.

### Biological Assays

Potency Assay: pERK

The purpose of this assay is to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO2 incubator. Test compounds were prepared in 10, 3-fold dilutions in DMSO, with a high concentration of 10 mM. On the day of assay, 40 nL of test compound was added to each well of cell culture plate using an Echo550 liquid handler (LabCyte®). Concentrations of test compound were tested in duplicate. After compound addition, cells were incubated 4 hours at 37° C., 5% CO2. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, plate was sealed, and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% paraformaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (pERK, CST-4370, Cell Signaling Technology) was diluted 1:200 in blocking buffer, and 50 μL was added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a LI-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

The following compounds exhibited a pERK EC50 of under 5 uM (H358 KRAS G12C): A48, A15, A272, A174, A163, A453, A447, A279, A240, A214, A225, A136, A226, A219, A228, A21, A12, A78, A424, A219, A378, A224, A4, A53, A187, A218, A213, A314, A220, A208, A24, A9, A126, A345, A46, A203, A210, A184, A 469, A366, A113, A328, A693, A639, A364, A100, A249, A486, A307, A347, A33, A210, A192, A285, A468, A185, A 612, A109, A284, A200, A2, A6, A606, A325, A139, A496, A393, A561, A125, A494, A547, A215, A258, A195, A259, A212, A637, A53, A63, A68, A178, A189, A205, A78, A254, A690, A563, A14, A19, A92, A576, A278, A331, A42, A6 7, A209, A350, A562, A652, A703, A623, A191, A241, A199, A193, A478, A251, A177, A222, A23, A59, A26, A211, A106, A279, A120, A7, A134, A521, A116, A467, A694, A729, A151, A110, A277, A340, A221, A723, A13, A442, A6 11, A50, A190, A553, A696, A211, A303, A613, A37, A146, A666, A688, A216, A390, A548, A238, A160, A183, A164, A451, A481, A524, A1, A186, A37, A635, A71, A269, A289, A489, A400, A731, A497, A568, A274, A253, A471, A 720, A241, A179, A180, A426, A117, A363, A716, A423, A217, A708, A227, A3, A12, A8, A381, A84, A408, A85, A1 71, A263, A473, A258, A564, A118, A103, A565, A641, A655, A47, A11, A392, A169, A487, A640, A206, A449, A35 8, A192, A148, A4, A41, A5, A18, A301, A10, A65, A554, A159, A264, A99, A79, A142, A143, A25, A98, A80, A101, A 730, A212, A359, A61, A441, A283, A413, A717, A145, A182, A62, A181, A233, A232, A634, A495, A34, A251, A53 9, A632, A54, A327, A37, A196, A607, A645, A35, A214, A225, A638, A40, A52, A268, A448, A575, A176, A593, A1 5, A17, A94, A170, A713, A93, A402, A64, A261, A399, A422, A214, A225, A625, A31, A119, A135, A281, A676, A709, A81, A32, A633, A39, A646, A662, A124, A732, A320, A81, A187, A354, A45, A570, A165, A66, A20, A455, A43 1, A270, A250, A457, A153, A404, A710, A541, A127, A373, A369, A557, A349, A598, A618, A60, A636, A499, A87, A156, A680, A477, A406, A330, A202, A535, A617, A737, A201, A302, A722, A209, A374, A631, A29, A555, A420, A380, A111, A306, A173, A628, A672, A51, A167, A588, A512, A194, A282, A412, A701, A583, A396, A678, A649, A27, A204, A626, A257, A614, A409, A172, A372, A353, A58, A728, A74, A619, A144, A183, A538, A445, A531, A3 60, A361, A459, A536, A344, A267, A574, A677, A530, A415, A30, A73, A152, A490, A702, A714, A483, A567, A43, A310, A319, A86, A321, A656, A739, A115, A130, A155, A608, A648, A168, A485, A738, A129, A650, A715, A488, A147, A121, A470, A115, A133, A510, A421, A309, A335, A387, A386, A734, A95, A430, A604, A458, A592, A384, A664, A197, A725, A89, A83, A586, A622, A305, A498, A668, A427, A630, A158, A644, A735, A70, A683, A352, A3 41, A719, A674, A70, A44, A501, A438, A698, A377, A417, A154, A433, A104, A184, A603, A280, A712, A237, A10 5, A394, A605, A517, A704, A566, A77, A356, A454, A600, A643, A112, A569, A529, A247, A463, A437, A718, A47 2, A461, A558, A48, A671, A395, A670, A681, A687, A382, A82, A686, A342, A436, A296, A16, A545, A533, A416, A149, A207, A371, A596, A675, A132, A419, A56, A579, A733, A573, A707, A597, A697, A75, A653, A362, A615, A 332, A69, A162, A128, A432, A654, A22, A397, A526, A582, A418, A91, A260, A97, A191, A55, A581, A375, A522, A108, A367, A610, A552, A571, A57, A543, A661, A138, A196, A246, A337, A446, A265, A96, A509, A123, A627, A 651, A682, A157, A572, A624, A691, A532, A462, A580, A695, A186, A316, A540, A590, A665, A244, A166, A587, A629, A595, A518, A519, A131, A502, A726, A452, A141, A181, A262, A338, A155, A389, A124, A275, A414, A546, A679, A425, A669, A28, A520, A88, A131, A589, A621, A182, A297, A594, A283, A194, A250, A336, A706, A252, A440, A107, A724, A525, A388, A175, A300, A333, A659, A346, A150, A476, A368, A528, A503, A504, A505, A684, A76, A736, A551, A383, A491, A492, A493, A410, A316, A295, A559, A511, A38, A140, A663, A334, A700, A692, A348, A584, A513, A657, A328, A515, A317, A135, A660, A351, A544, A281, A685, A602, A556, A385, A326, A464, A465, A403, A133, A299, A667, A255, A334, A256, A585, A642, A133, A443, A435, A560, A444, A439, A324, A12 0, A407, A527, A245, A370, A537, A247, A474, A475, A705, A323, A112, A298, A609, A673, A292, A599, A132, A1 45, A266, A601, A466, A549, A379, A727, A167, A711, A75, A76, A121, A357, A620, A316, A479, A290, A339, A32 2, A376, A456, A391, A291, A550, A343, A721, A689, A411, A578, A616, A534, A365, A658, A699, A577, A647, A5 91, A542, A279, A294.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μL) were transferred to the next wells containing 30 μL of 100% DMSO, and repeated until a 9-concentration 3-fold serial dilution was made (starting assay concentration of 10 μM). Test compounds (132.5 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data is reported as IC50 values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} were combined in a 384-well assay plate at final concentrations of 25 μM, 12.5 nM and 50 nM, respectively. Compound was present in plate wells as a 10-point 3-fold dilution series starting at a final concentration of 30 μM. After incubation at 25° C. for 3 hours, a mixture of Anti-His Eu-W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras:RAF complex were identified as those eliciting a decrease in the TR-FRET ratio relative to DMSO control wells.

TABLE 4
Biological Assay Data for Representative
Compounds of the Present Invention

| | | H358 Cell | | |
|---|---|---|---|---|
| | H358 pERK | Viability | FRET | FRET |
| | (K-Ras G12C) | (K-Ras G12C) | (K-Ras G12C) | (K-Ras G13C) |
| Ex# | EC50, uM | IC50, uM | IC50, uM | IC50, uM |
| A208 | 0.013 | 0.004 | 0.023 | 7.48 |
| A193 | 0.067 | 0.054 | 0.295 | 10.2 |
| A186 | 0.013 | 0.005 | 0.038 | 0.94 |
| A89a | 0.003 | 0.0008 | 0.009 | 0.82 |
| A89b | 0.028 | 0.011 | 0.034 | 0.58 |

TABLE 5
Additional H358 Cell Viability assay
data (K-Ras G12C, IC50, uM):

| IC50* | Examples |
|---|---|
| + | A104, A107, A108, A112, A120, A121, A123, A124, |
| | A128, A131, A131, A132, A133, A133, A135, A138, |
| | A140, A141, A145, A150, A155, A157, A162, A166, |
| | A167, A175, A181, A182, A183, A186, A191, A194, |
| | A196, A237, A244, A245, A246, A247, A250, A255, |
| | A256, A260, A266, A275, A28, A281, A283, A290, |
| | A291, A292, A295, A296, A298, A299, A300, A309, |
| | A316, A316, A316, A317, A322, A323, A324, A326, |
| | A328, A332, A333, A334, A334, A336, A337, A339, |
| | A343, A344, A346, A348, A351, A352, A356, A357, |
| | A365, A368, A370, A371, A375, A376, A377, A379, |
| | A38, A380, A382, A383, A384, A385, A388, A389, |
| | A391, A394, A395, A396, A397, A403, A407, A410, |
| | A411, A414, A416, A419, A425, A427, A435, A439, |
| | A440, A443, A444, A445, A446, A452, A454, A456, |
| | A461, A462, A463, A464, A465, A466, A472, A474, |
| | A475, A476, A479, A483, A485, A488, A491, A492, |
| | A493, A502, A503, A504, A505, A509, A511, A513, |
| | A515, A517, A518, A519, A520, A522, A525, A526, |
| | A527, A528, A532, A533, A534, A537, A540, A542, |
| | A543, A544, A546, A549, A55, A550, A551, A552, |
| | A556, A559, A56, A560, A566, A567, A57, A571, |
| | A572, A577, A578, A579, A581, A584, A585, A587, |
| | A589, A590, A591, A592, A594, A595, A596, A597, |
| | A599, A600, A601, A602, A604, A609, A610, A615, |
| | A616, A620, A621, A624, A627, A629, A631, A636, |
| | A642, A643, A644, A647, A657, A658, A659, A660, |
| | A661, A663, A665, A667, A673, A674, A675, A677, |
| | A684, A685, A686, A689, A691, A692, A699, A700, |
| | A704, A705, A706, A707, A711, A712, A714, A718, |
| | A721, A724, A726, A727, A733, A735, A736, A738, |
| | A75, A76, A76, A88, A91, A96 |
| ++ | A10, A105, A111, A112, A115, A115, A119, A12, |
| | A121, A124, A129, A130, A132, A133, A135, A143, |
| | A144, A145, A147, A149, A15, A152, A154, A155, |
| | A156, A158, A16, A165, A167, A168, A172, A173, |
| | A176, A181, A182, A184, A187, A194, A197, A20, |
| | A201, A202, A204, A207, A209, A212, A214, A214, |
| | A22, A225, A225, A232, A233, A238, A241, A247, |
| | A250, A251, A252, A257, A261, A262, A264, A265, |
| | A267, A268, A269, A27, A270, A280, A281, A282, |
| | A283, A297, A30, A302, A305, A306, A31, A310, |
| | A319, A320, A321, A327, A330, A335, A338, A34, |
| | A341, A342, A349, A35, A353, A354, A358, A359, |
| | A360, A361, A362, A363, A367, A369, A37, A372, |
| | A373, A374, A381, A386, A387, A39, A392, A393, |
| | A399, A40, A400, A402, A404, A406, A408, A409, |
| | A41, A412, A413, A415, A417, A418, A420, A421, |
| | A422, A423, A426, A43, A430, A431, A432, A433, |
| | A436, A437, A438, A44, A441, A442, A448, A449, |
| | A45, A451, A455, A457, A458, A459, A467, A468, |
| | A47, A470, A471, A473, A477, A478, A48, A481, |
| | A487, A489, A490, A495, A497, A498, A499, A5, |
| | A501, A51, A510, A512, A52, A524, A529, A530, |
| | A531, A535, A536, A538, A54, A541, A545, A555, |
| | A558, A564, A565, A568, A569, A573, A574, A575, |
| | A58, A580, A582, A583, A586, A588, A598, A60, |
| | A603, A605, A607, A608, A61, A611, A613, A614, |
| | A617, A618, A619, A62, A622, A623, A625, A626, |
| | A628, A630, A633, A634, A637, A638, A64, A640, |
| | A641, A645, A646, A648, A649, A650, A651, A653, |
| | A654, A655, A656, A66, A662, A664, A666, A668, |
| | A669, A670, A671, A672, A676, A678, A679, A680, |
| | A681, A682, A683, A687, A69, A694, A695, A697, |
| | A698, A70, A70, A701, A702, A703, A708, A709, |
| | A710, A713, A715, A716, A717, A719, A720, A722, |
| | A723, A725, A728, A73, A730, A732, A734, A737, |
| | A739, A74, A75, A77, A79, A81, A82, A83, A86, |
| | A87, A89, A93, A94, A95, A97, A98, A99 |
| +++ | A1, A100, A101, A103, A106, A109, A11, A110, |
| | A113, A114, A116, A117, A118, A120, A127, A13, |
| | A134, A139, A14, A142, A144, A146, A148, A151, |
| | A153, A159, A160, A164, A169, A17, A170, A171, |
| | A177, A18, A183, A185, A186, A19, A190, A191, |
| | A192, A192, A193, A195, A196, A199, A200, A205, |
| | A206, A209, A210, A211, A211, A212, A215, A216, |
| | A217, A219, A219, A221, A222, A226, A227, A23, |
| | A241, A249, A25, A251, A253, A254, A258, A258, |
| | A259, A26, A263, A274, A277, A278, A279, A284, |
| | A289, A29, A3, A301, A303, A304, A304, A304, |
| | A304, A308, A314, A32, A325, A328, A329, A33, |
| | A331, A340, A345, A347, A350, A355, A364, A366, |
| | A37, A37, A378, A390, A398, A4, A401, A42, A424, |
| | A447, A453, A469, A482, A484, A486, A494, A496, |
| | A50, A506, A507, A508, A514, A521, A523, A53, |
| | A53, A539, A547, A548, A553, A554, A557, A561, |
| | A562, A563, A570, A576, A59, A593, A6, A606, |
| | A612, A63, A632, A635, A639, A65, A652, A67, |
| | A68, A688, A690, A693, A696, A7, A71, A729, |
| | A731, A78, A8, A80, A81, A84, A85, A9, A92 |
| ++++ | A102, A12, A122, A125, A126, A136, A137, A148, |
| | A15, A161, A163, A174, A178, A179, A180, A184, |
| | A187, A188, A189, A198, A2, A203, A208, A21, |
| | A210, A210, A210, A211, A211, A213, A213, A214, |
| | A214, A215, A216, A217, A218, A220, A220, A221, |
| | A222, A223, A223, A224, A224, A225, A225, A228, |
| | A229, A230, A231, A231, A231, A234, A235, A236, |
| | A239, A239, A24, A240, A242, A243, A248, A261, |
| | A271, A272, A273, A276, A279, A279, A283, A285, |
| | A286, A287, A288, A293, A307, A311, A312, A313, |
| | A318, A318, A32, A4, A405, A428, A429, A450, |
| | A46, A460, A48, A480, A49, A500, A516, A72, A78, |
| | A90 |

*Key:
++++: IC50 ≥ 1 uM
+++: 1 uM > IC50 ≥ 0.1 uM
++: 0.1 uM > IC50 ≥ 0.01 uM
+: IC50 < 0.01 uM

Additional Ras-Raf disruption/FRET/MOA assay data (IC50, μM):

### *Key:

+++++:IC50≥10 μM

++++:10 μM>IC50≥1 μM

+++:1 μM>IC50≥0.1 μM

++:0.1 μM>IC50≥0.01 μM

+: <0.01 μM

TABLE 6
KRAS G12S FRET data

| IC50* | Examples |
|---|---|
| + | none |
| ++ | A028, A075, A076, A076, A087, A112, A145, A155, |
| | A167, A181, A183, A184, A194, A233, A255, A256, |
| | A260, A262, A264, A265, A266, A268, A270, A275, |
| | A292, A294, A295, A298, A299, A300, A319, A333, |
| | A334, A334, A338, A367, A382, A383, A385, A388, |
| | A389, A418, A433, A464, A465, A479, A491, A492, |
| | A493, A527, A537, A542, A577, A596, A598, A602, |
| | A609, A621, A629, A664, A665, A679, A712, A734, |
| | A736 |
| +++ | A004, A005, A012, A015, A016, A022, A027, A029, |
| | A032, A034, A037, A037, A041, A043, A044, A047, |
| | A048, A051, A052, A054, A055, A056, A057, A058, |
| | A064, A070, A070, A072, A074, A077, A079, A082, |
| | A083, A086, A088, A091, A093, A095, A096, A097, |
| | A101, A103, A105, A107, A123, A127, A128, A129, |
| | A131, A132, A135, A141, A147, A148, A149, A150, |
| | A153, A154, A158, A164, A166, A178, A182, A189, |
| | A193, A194, A196, A206, A207, A237, A241, A244, |
| | A245, A246, A247, A250, A251, A252, A257, A261, |
| | A261, A263, A267, A269, A280, A281, A281, A296, |
| | A297, A305, A306, A328, A336, A337, A349, A350, |
| | A351, A353, A354, A357, A360, A361, A368, A369, |
| | A379, A384, A386, A387, A393, A394, A395, A397, |
| | A406, A407, A408, A409, A410, A415, A416, A417, |
| | A419, A420, A421, A430, A431, A432, A435, A436, |
| | A437, A438, A443, A455, A463, A470, A490, A502, |
| | A515, A517, A518, A519, A529, A536, A538, A539, |
| | A541, A543, A544, A545, A548, A554, A555, A557, |
| | A558, A560, A564, A570, A571, A572, A573, A575, |
| | A578, A580, A581, A582, A583, A586, A591, A594, |
| | A603, A604, A605, A606, A608, A611, A612, A618, |
| | A620, A622, A624, A625, A630, A631, A632, A633, |
| | A634, A635, A636, A650, A651, A653, A654, A655, |
| | A656, A659, A661, A662, A667, A668, A669, A670, |
| | A671, A672, A677, A678, A680, A681, A682, A683, |
| | A684, A685, A686, A687, A689, A690, A695, A696, |
| | A697, A698, A699, A700, A701, A702, A704, A709, |
| | A710, A711, A713, A714, A717, A718, A719, A728, |
| | A729, A730, A731, A732, A737 |
| ++++ | A001, A003, A006, A007, A008, A010, A013, A014, |
| | A017, A018, A019, A020, A025, A030, A031, A033, |
| | A035, A036, A038, A039, A040, A045, A050, A053, |
| | A060, A061, A062, A063, A066, A067, A068, A069, |
| | A071, A073, A075, A081, A081, A089, A092, A098, |
| | A099, A104, A108, A109, A110, A111, A112, A113, |
| | A115, A115, A117, A119, A120, A121, A124, A125, |
| | A131, A132, A133, A133, A134, A135, A137, A140, |
| | A143, A145, A146, A151, A155, A159, A161, A162, |
| | A167, A168, A169, A172, A173, A175, A176, A179, |
| | A180, A181, A182, A183, A184, A186, A187, A197, |
| | A199, A202, A204, A209, A210, A211, A212, A213, |
| | A214, A214, A215, A216, A217, A221, A223, A225, |
| | A225, A226, A227, A238, A241, A247, A249, A250, |
| | A251, A253, A254, A258, A258, A259, A277, A278, |
| | A282, A283, A290, A291, A301, A302, A309, A310, |
| | A314, A317, A320, A321, A324, A325, A326, A327, |
| | A330, A331, A332, A335, A339, A340, A341, A342, |
| | A343, A344, A347, A348, A356, A358, A359, A363, |
| | A364, A365, A366, A370, A371, A372, A373, A376, |
| | A378, A380, A381, A390, A391, A392, A396, A399, |
| | A401, A402, A411, A412, A413, A414, A422, A423, |
| | A424, A425, A426, A427, A439, A440, A442, A444, |
| | A445, A446, A447, A449, A451, A452, A454, A456, |
| | A457, A458, A459, A461, A462, A466, A468, A469, |
| | A471, A472, A473, A474, A475, A476, A481, A485, |
| | A487, A488, A489, A495, A498, A499, A501, A503, |
| | A504, A505, A506, A507, A509, A510, A511, A512, |
| | A513, A520, A521, A522, A525, A526, A528, A530, |
| | A531, A532, A533, A534, A535, A540, A546, A547, |
| | A549, A550, A551, A552, A553, A559, A561, A562, |
| | A563, A565, A566, A567, A568, A569, A574, A579, |
| | A585, A588, A590, A592, A593, A595, A597, A600, |
| | A601, A607, A610, A616, A619, A623, A626, A628, |
| | A637, A638, A639, A640, A641, A642, A645, A647, |
| | A652, A658, A660, A673, A676, A688, A691, A692, |
| | A693, A694, A705, A706, A707, A716, A720, A722, |
| | A723, A725, A726, A727, A733, A738, A739 |
| +++++ | A002, A004, A009, A011, A012, A015, A021, A023, |
| | A024, A026, A032, A036, A037, A038, A042, A046, |
| | A048, A049, A053, A059, A065, A078, A078, A080, |
| | A084, A085, A090, A094, A100, A102, A106, A114, |
| | A116, A118, A120, A121, A122, A124, A126, A130, |
| | A133, A136, A138, A139, A142, A144, A144, A148, |
| | A152, A156, A157, A160, A163, A165, A170, A171, |
| | A174, A177, A185, A186, A187, A188, A190, A191, |
| | A191, A192, A192, A195, A196, A198, A200, A201, |
| | A203, A205, A208, A209, A210, A210, A210, A211, |
| | A211, A211, A212, A213, A214, A214, A215, A216, |
| | A217, A218, A218, A219, A219, A219, A219, A220, |
| | A220, A221, A222, A222, A223, A224, A224, A225, |
| | A225, A228, A229, A230, A231, A231, A231, A232, |
| | A234, A235, A236, A239, A239, A240, A242, A243, |
| | A248, A271, A272, A273, A274, A276, A279, A279, |
| | A279, A283, A283, A284, A285, A286, A287, A288, |
| | A289, A293, A303, A304, A304, A304, A304, A307, |
| | A308, A311, A312, A313, A316, A316, A316, A318, |
| | A318, A322, A323, A328, A329, A345, A346, A352, |
| | A355, A362, A374, A375, A377, A398, A400, A403, |
| | A404, A405, A428, A429, A441, A448, A450, A453, |
| | A460, A467, A477, A478, A480, A482, A483, A484, |
| | A486, A494, A496, A497, A500, A508, A514, A516, |
| | A523, A524, A556, A576, A584, A587, A589, A599, |
| | A613, A614, A615, A617, A627, A643, A644, A646, |
| | A648, A649, A657, A663, A666, A674, A675, A703, |
| | A708, A715, A721, A724, A735 |

+++++: IC50 ≥ 10 uM
++++: 10 uM > IC50 ≥ 1 uM
+++: 1 uM > IC50 ≥ 0.1 uM
++: 0.1 uM > IC50 ≥ 0.01 uM
+: IC50 < 0.01 uM

TABLE 7
KRAS G12D FRET data

| IC50* | Examples |
|---|---|
| + | none |
| ++ | A183, A264, A265, A268, A319, A388, A464, A465, |
| | A664 |
| +++ | A028, A054, A075, A076, A076, A087, A107, A112, |
| | A123, A131, A145, A153, A155, A167, A184, A189, |
| | A194, A233, A247, A255, A256, A257, A260, A261, |
| | A262, A263, A266, A269, A270, A275, A292, A294, |
| | A295, A298, A300, A333, A334, A334, A338, A349, |
| | A353, A354, A367, A368, A382, A383, A384, A385, |
| | A386, A387, A389, A394, A407, A409, A417, A418, |
| | A432, A433, A436, A479, A491, A492, A493, A527, |
| | A529, A537, A542, A555, A577, A578, A594, A596, |
| | A598, A602, A603, A605, A608, A609, A612, A621, |
| | A622, A629, A633, A650, A651, A653, A654, A662, |
| | A665, A667, A669, A671, A678, A679, A681, A682, |
| | A683, A685, A696, A697, A698, A700, A701, A704, |
| | A709, A710, A711, A712, A719, A734, A736, A737 |
| ++++ | A005, A007, A012, A015, A016, A022, A029, A034, |
| | A037, A037, A038, A038, A039, A043, A044, A046, |
| | A047, A048, A051, A052, A055, A056, A057, A058, |
| | A067, A070, A070, A071, A072, A074, A077, A079, |
| | A082, A086, A088, A090, A091, A093, A096, A097, |
| | A101, A103, A105, A108, A110, A116, A120, A120, |
| | A121, A121, A127, A128, A129, A130, A131, A132, |
| | A133, A133, A133, A134, A135, A138, A139, A140, |
| | A141, A142, A144, A144, A147, A148, A149, A150, |
| | A152, A154, A155, A156, A157, A162, A163, A164, |
| | A166, A175, A178, A179, A180, A181, A182, A183, |
| | A194, A196, A207, A213, A218, A218, A220, A224, |
| | A224, A237, A241, A244, A245, A246, A247, A250, |
| | A251, A252, A253, A254, A258, A261, A267, A280, |
| | A281, A281, A282, A296, A297, A299, A305, A306, |
| | A314, A326, A328, A330, A332, A336, A337, A341, |
| | A342, A350, A351, A356, A357, A358, A360, A361, |
| | A365, A366, A369, A370, A373, A379, A380, A381, |
| | A390, A393, A395, A396, A397, A406, A408, A410, |
| | A411, A412, A415, A416, A419, A420, A421, A422, |
| | A430, A431, A435, A437, A438, A440, A443, A444, |
| | A451, A454, A455, A456, A459, A463, A470, A476, |
| | A487, A488, A490, A499, A501, A502, A503, A504, |
| | A505, A510, A513, A515, A517, A518, A519, A520, |
| | A525, A534, A536, A538, A539, A541, A543, A544, |
| | A545, A546, A547, A548, A550, A551, A552, A553, |
| | A554, A557, A558, A559, A560, A561, A562, A564, |
| | A566, A567, A570, A571, A572, A573, A575, A580, |
| | A581, A582, A583, A586, A588, A591, A593, A595, |
| | A600, A604, A606, A607, A610, A611, A618, A619, |
| | A620, A623, A624, A625, A626, A630, A631, A632, |
| | A634, A635, A636, A640, A645, A647, A655, A656, |
| | A658, A659, A660, A661, A668, A670, A672, A673, |
| | A676, A677, A680, A684, A686, A687, A688, A689, |
| | A690, A691, A694, A695, A699, A702, A707, A713, |
| | A714, A717, A718, A722, A727, A728, A729, A730, |
| | A731, A732, A738, A739 |
| +++++ | A001, A002, A003, A004, A004, A006, A008, A009, |
| | A010, A011, A012, A013, A014, A015, A017, A018, |
| | A019, A020, A021, A023, A024, A025, A026, A027, |
| | A030, A031, A032, A032, A033, A035, A036, A036, |
| | A037, A040, A041, A042, A045, A048, A049, A050, |
| | A053, A053, A059, A060, A061, A062, A063, A064, |
| | A065, A066, A068, A069, A073, A075, A078, A078, |
| | A080, A081, A081, A083, A084, A085, A089, A092, |
| | A094, A095, A098, A099, A100, A102, A104, A106, |
| | A109, A111, A112, A113, A114, A115, A115, A117, |
| | A118, A119, A122, A124, A124, A125, A126, A132, |
| | A135, A136, A137, A143, A145, A146, A148, A151, |
| | A158, A159, A160, A161, A165, A167, A168, A169, |
| | A170, A171, A172, A173, A174, A176, A177, A181, |
| | A182, A184, A185, A186, A186, A187, A187, A188, |
| | A190, A191, A191, A192, A192, A193, A195, A196, |
| | A197, A198, A199, A200, A201, A202, A203, A204, |
| | A205, A206, A208, A209, A209, A210, A210, A210, |
| | A210, A211, A211, A211, A211, A212, A212, A213, |
| | A214, A214, A214, A214, A215, A215, A216, A216, |
| | A217, A217, A219, A219, A219, A219, A220, A221, |
| | A221, A222, A222, A223, A223, A225, A225, A225, |
| | A225, A226, A227, A228, A229, A230, A231, A231, |
| | A231, A232, A234, A235, A236, A238, A239, A239, |
| | A240, A241, A242, A243, A248, A249, A250, A251, |
| | A258, A259, A271, A272, A273, A274, A276, A277, |
| | A278, A279, A279, A279, A283, A283, A283, A284, |
| | A285, A286, A287, A288, A289, A290, A291, A293, |
| | A301, A302, A303, A304, A304, A304, A304, A307, |
| | A308, A309, A310, A311, A312, A313, A316, A316, |
| | A316, A317, A318, A318, A320, A321, A322, A323, |
| | A324, A325, A327, A328, A329, A331, A335, A339, |
| | A340, A343, A344, A345, A346, A347, A348, A352, |
| | A355, A359, A362, A363, A364, A371, A372, A374, |
| | A375, A376, A377, A378, A391, A392, A398, A399, |
| | A400, A401, A402, A403, A404, A405, A413, A414, |
| | A423, A424, A425, A426, A427, A428, A429, A439, |
| | A441, A442, A445, A446, A447, A448, A449, A450, |
| | A452, A453, A457, A458, A460, A461, A462, A466, |
| | A467, A468, A469, A471, A472, A473, A474, A475, |
| | A477, A478, A480, A481, A482, A483, A484, A485, |
| | A486, A489, A494, A495, A496, A497, A498, A500, |
| | A506, A507, A508, A509, A511, A512, A514, A516, |
| | A521, A522, A523, A524, A526, A528, A530, A531, |
| | A532, A533, A535, A540, A549, A556, A563, A565, |
| | A568, A569, A574, A576, A579, A584, A585, A587, |
| | A589, A590, A592, A597, A599, A601, A613, A614, |
| | A615, A616, A617, A627, A628, A637, A638, A639, |
| | A641, A642, A643, A644, A646, A648, A649, A652, |
| | A657, A663, A666, A674, A675, A692, A693, A703, |
| | A705, A706, A708, A715, A716, A720, A721, A723, |
| | A724, A725, A726, A733, A735 |

TABLE 8
KRAS G13C FRET data

| IC50* | Examples |
|---|---|
| + | A088, A096, A097, A107, A131, A132, A155, A175, |
| | A247, A250, A251, A252, A253, A255, A258, A262, |
| | A270, A292, A294, A297, A298, A299, A334, A336, |
| | A338, A474, A475, A529, A542, A683 |
| ++ | A031, A037, A038, A046, A067, A069, A071, A075, |
| | A076, A076, A090, A108, A112, A116, A121, A123, |
| | A124, A131, A132, A133, A133, A135, A140, A141, |
| | A145, A150, A155, A163, A167, A172, A178, A182, |
| | A183, A194, A196, A218, A224, A233, A244, A245, |
| | A246, A247, A249, A250, A251, A254, A256, A257, |
| | A258, A259, A260, A261, A263, A264, A265, A266, |
| | A267, A268, A275, A280, A281, A283, A295, A296, |
| | A300, A305, A306, A319, A333, A334, A337, A379, |
| | A383, A385, A388, A389, A394, A396, A397, A417, |
| | A418, A421, A432, A433, A438, A452, A464, A465, |
| | A479, A491, A492, A493, A517, A525, A527, A537, |
| | A538, A539, A540, A541, A545, A546, A552, A553, |
| | A554, A555, A564, A570, A571, A572, A573, A574, |
| | A575, A577, A578, A591, A594, A595, A597, A600, |
| | A602, A603, A604, A605, A606, A607, A608, A609, |
| | A610, A621, A622, A629, A630, A632, A650, A664, |
| | A668, A679, A680, A695, A698, A699, A706, A711, |
| | A714, A718, A719, A734, A736, A737 |
| +++ | A005, A012, A013, A016, A018, A022, A027, A028, |
| | A029, A033, A036, A037, A038, A043, A044, A047, |
| | A048, A049, A052, A054, A055, A056, A057, A070, |
| | A070, A072, A074, A077, A082, A086, A087, A091, |
| | A093, A095, A101, A105, A110, A117, A120, A125, |
| | A126, A127, A128, A129, A130, A135, A138, A142, |
| | A146, A147, A149, A153, A157, A158, A162, A164, |
| | A165, A166, A179, A180, A181, A184, A189, A196, |
| | A199, A207, A209, A213, A216, A218, A220, A220, |
| | A222, A224, A232, A237, A238, A241, A241, A261, |
| | A269, A277, A281, A302, A314, A322, A323, A324, |
| | A328, A332, A340, A341, A342, A344, A349, A350, |
| | A351, A353, A354, A356, A357, A358, A360, A361, |
| | A365, A366, A367, A368, A369, A370, A371, A372, |
| | A373, A380, A382, A384, A386, A387, A390, A393, |
| | A395, A401, A406, A407, A408, A409, A410, A411, |
| | A412, A415, A416, A419, A420, A422, A423, A425, |
| | A430, A431, A435, A436, A437, A439, A440, A443, |
| | A444, A445, A449, A451, A454, A455, A457, A458, |
| | A459, A463, A466, A470, A471, A476, A485, A487, |
| | A488, A490, A497, A501, A502, A503, A504, A505, |
| | A509, A510, A513, A515, A518, A519, A524, A534, |
| | A536, A543, A544, A547, A549, A550, A551, A557, |
| | A558, A559, A560, A561, A562, A563, A566, A567, |
| | A576, A580, A581, A582, A583, A586, A593, A596, |
| | A598, A601, A611, A612, A618, A619, A620, A624, |
| | A625, A631, A633, A634, A635, A636, A640, A642, |
| | A645, A647, A651, A652, A653, A654, A655, A658, |
| | A659, A660, A661, A662, A665, A667, A669, A670, |
| | A671, A672, A673, A678, A681, A682, A684, A685, |
| | A686, A687, A688, A689, A690, A692, A694, A696, |
| | A697, A700, A701, A702, A704, A707, A709, A710, |
| | A712, A717, A722, A725, A726, A727, A728, A729, |
| | A730, A731, A732, A733, A738, A739 |
| ++++ | A001, A003, A004, A006, A007, A008, A010, A014, |
| | A015, A017, A019, A020, A025, A030, A032, A034, |
| | A035, A039, A040, A041, A045, A051, A058, A060, |
| | A061, A063, A064, A068, A073, A075, A078, A078, |
| | A079, A081, A081, A083, A089, A092, A098, A099, |
| | A103, A104, A111, A112, A115, A115, A119, A120, |
| | A121, A124, A133, A134, A139, A144, A144, A145, |
| | A148, A152, A154, A156, A161, A167, A168, A169, |
| | A170, A171, A174, A176, A181, A182, A183, A186, |
| | A187, A191, A193, A194, A197, A200, A201, A204, |
| | A206, A212, A213, A214, A214, A214, A217, A221, |
| | A225, A225, A225, A227, A235, A242, A278, A279, |
| | A282, A283, A283, A284, A289, A290, A291, A301, |
| | A307, A308, A309, A310, A316, A316, A317, A320, |
| | A321, A325, A326, A327, A330, A331, A335, A339, |
| | A343, A345, A346, A347, A348, A359, A362, A363, |
| | A364, A375, A376, A377, A378, A381, A391, A392, |
| | A398, A399, A400, A402, A403, A413, A414, A424, |
| | A426, A427, A441, A442, A446, A447, A456, A460, |
| | A461, A462, A467, A468, A469, A472, A473, A477, |
| | A480, A481, A483, A484, A486, A489, A494, A495, |
| | A496, A498, A499, A506, A507, A508, A511, A512, |
| | A514, A516, A520, A521, A522, A526, A528, A530, |
| | A531, A532, A533, A535, A548, A556, A565, A568, |
| | A569, A579, A584, A585, A588, A589, A590, A592, |
| | A599, A614, A616, A623, A626, A628, A637, A638, |
| | A639, A641, A643, A644, A648, A656, A657, A663, |
| | A674, A676, A677, A691, A693, A703, A705, A708, |
| | A713, A716, A720, A721, A723, A735 |
| +++++ | A210, A173, A109, A042, A062, A137, A303, A228, |
| | A666, A214, A225, A627, A675, A229, A015, A066, |
| | A184, A209, A328, A288, A011, A587, A448, A613, |
| | A617, A226, A240, A316, A113, A059, A002, A374, |
| | A223, A313, A190, A212, A219, A065, A032, A615, |
| | A143, A215, A080, A026, A050, A191, A219, A216, |
| | A217, A106, A649, A151, A053, A478, A222, A219, |
| | A037, A211, A404, A724, A085, A248, A202, A122, |
| | A159, A094, A004, A009, A012, A021, A023, A024, |
| | A036, A048, A053, A084, A100, A102, A114, A118, |
| | A136, A148, A160, A177, A185, A186, A187, A188, |
| | A192, A192, A195, A198, A203, A205, A208, A210, |
| | A210, A210, A211, A211, A211, A215, A219, A221, |
| | A223, A230, A231, A231, A231, A234, A236, A239, |
| | A239, A243, A271, A272, A273, A274, A276, A279, |
| | A279, A285, A286, A287, A293, A304, A304, A304, |
| | A304, A311, A312, A318, A318, A329, A352, A355, |
| | A405, A428, A429, A450, A453, A482, A500, A523, |
| | A646, A715 |

TABLE 9
KRAS G12V FRET data

| IC50* | Examples |
|---|---|
| + | None |
| ++ | A028, A075, A076, A076, A087, A112, A132, A145, A155, |
| | A167, A183, A184, A194, A233, A245, A262, A264, A265, |
| | A266, A268, A275, A292, A295, A298, A300, A305, A319, |
| | A333, A334, A338, A368, A383, A388, A389, A418, A464, |
| | A465, A479, A491, A492, A493, A527, A537, A542, A602, |
| | A609, A621, A629, A665, A679, A734 |
| +++ | A004, A005, A012, A015, A016, A022, A027, A029, A034, |
| | A037, A037, A041, A043, A044, A047, A048, A051, A052, |
| | A054, A055, A056, A057, A058, A069, A070, A072, A074, |
| | A077, A079, A082, A086, A088, A091, A093, A095, A096, |
| | A097, A101, A103, A105, A107, A123, A127, A128, A129, |
| | A131, A135, A141, A147, A148, A149, A150, A153, A154, |
| | A155, A162, A164, A166, A178, A180, A181, A182, A183, |
| | A189, A193, A194, A196, A206, A207, A209, A237, A238, |
| | A241, A244, A246, A247, A250, A251, A252, A253, A255, |
| | A256, A257, A258, A260, A261, A263, A267, A269, A270, |
| | A280, A281, A281, A294, A296, A297, A299, A306, A334, |
| | A336, A337, A349, A350, A353, A354, A357, A360, A361, |
| | A366, A367, A369, A373, A379, A380, A382, A384, A385, |
| | A386, A387, A394, A395, A397, A406, A407, A408, A409, |
| | A410, A415, A416, A417, A419, A420, A421, A431, A432, |
| | A433, A435, A436, A437, A438, A443, A444, A463, A490, |
| | A517, A518, A519, A529, A536, A538, A539, A543, A544, |
| | A545, A546, A548, A550, A554, A555, A564, A570, A571, |
| | A572, A577, A578, A580, A581, A582, A586, A591, A594, |
| | A596, A598, A603, A604, A605, A606, A608, A611, A612, |
| | A618, A620, A622, A624, A625, A630, A631, A632, A633, |
| | A634, A635, A650, A651, A653, A654, A655, A659, A660, |
| | A662, A664, A667, A668, A669, A670, A671, A672, A673, |
| | A678, A680, A681, A682, A683, A684, A685, A686, A687, |
| | A695, A696, A697, A698, A699, A700, A701, A702, A704, |
| | A709, A710, A711, A712, A714, A717, A718, A719, A728, |
| | A736, A737 |
| ++++ | A001, A003, A006, A007, A008, A010, A013, A017, A018, |
| | A025, A030, A031, A032, A035, A036, A038, A039, A040, |
| | A045, A046, A050, A060, A061, A062, A063, A064, A066, |
| | A067, A068, A070, A071, A073, A075, A080, A081, A081, |
| | A083, A089, A092, A098, A099, A104, A108, A109, A110, |
| | A111, A112, A113, A115, A115, A117, A120, A121, A124, |
| | A125, A131, A132, A133, A133, A134, A135, A137, A138, |
| | A140, A143, A145, A146, A151, A152, A158, A159, A161, |
| | A167, A168, A169, A172, A173, A175, A176, A179, A181, |
| | A182, A184, A186, A187, A191, A197, A199, A204, A210, |
| | A211, A212, A213, A215, A216, A217, A218, A221, A223, |
| | A224, A226, A227, A232, A241, A242, A247, A249, A251, |
| | A254, A258, A259, A261, A277, A278, A282, A283, A289, |
| | A290, A291, A301, A302, A307, A309, A310, A314, A316, |
| | A317, A320, A321, A324, A325, A326, A328, A330, A331, |
| | A332, A335, A339, A340, A341, A342, A343, A344, A346, |
| | A348, A351, A356, A358, A359, A362, A363, A364, A365, |
| | A370, A372, A375, A376, A378, A381, A390, A391, A392, |
| | A393, A396, A398, A399, A401, A402, A403, A411, A412, |
| | A413, A414, A422, A423, A424, A426, A427, A430, A439, |
| | A440, A445, A446, A447, A449, A451, A452, A454, A455, |
| | A456, A457, A458, A459, A461, A462, A466, A468, A469, |
| | A470, A472, A473, A474, A475, A476, A477, A481, A483, |
| | A485, A487, A488, A489, A495, A498, A499, A501, A502, |
| | A503, A504, A505, A506, A507, A509, A510, A511, A512, |
| | A513, A515, A520, A521, A522, A525, A526, A528, A530, |
| | A531, A532, A533, A534, A540, A541, A547, A549, A551, |
| | A552, A553, A556, A557, A558, A559, A560, A561, A562, |
| | A563, A565, A566, A567, A573, A574, A575, A579, A583, |
| | A584, A585, A588, A590, A592, A593, A595, A597, A599, |
| | A600, A601, A607, A610, A616, A619, A623, A626, A628, |
| | A636, A637, A638, A639, A640, A641, A642, A645, A647, |
| | A652, A656, A658, A661, A676, A677, A688, A689, A690, |
| | A691, A692, A693, A694, A703, A705, A706, A707, A708, |
| | A713, A716, A720, A721, A722, A723, A725, A726, A727, |
| | A729, A730, A731, A732, A733, A738, A739 |
| +++++ | A002, A004, A009, A011, A012, A014, A015, A019, A020, |
| | A021, A023, A024, A026, A032, A033, A036, A037, A038, |
| | A042, A048, A049, A053, A053, A059, A065, A078, A078, |
| | A084, A085, A090, A094, A100, A102, A106, A114, A116, |
| | A118, A119, A120, A121, A122, A124, A126, A130, A133, |
| | A136, A139, A142, A144, A144, A148, A156, A157, A160, |
| | A163, A165, A170, A171, A174, A177, A185, A186, A187, |
| | A188, A190, A191, A192, A192, A195, A196, A198, A200, |
| | A201, A202, A203, A205, A208, A209, A210, A210, A210, |
| | A211, A211, A211, A212, A213, A214, A214, A214, A214, |
| | A215, A216, A217, A218, A219, A219, A219, A219, A220, |
| | A220, A221, A222, A222, A223, A224, A225, A225, A225, |
| | A225, A228, A229, A230, A231, A231, A231, A234, A235, |
| | A236, A239, A239, A240, A243, A248, A250, A271, A272, |
| | A273, A274, A276, A279, A279, A279, A283, A283, A284, |
| | A285, A286, A287, A288, A293, A303, A304, A304, A304, |
| | A304, A308, A311, A312, A313, A316, A316, A318, A318, |
| | A322, A323, A327, A328, A329, A345, A347, A352, A355, |
| | A371, A374, A377, A400, A404, A405, A425, A428, A429, |
| | A441, A442, A448, A450, A453, A460, A467, A471, A478, |
| | A480, A482, A484, A486, A494, A496, A497, A500, A508, |
| | A514, A516, A523, A524, A535, A568, A569, A576, A587, |
| | A589, A613, A614, A615, A617, A627, A643, A644, A646, |
| | A648, A649, A657, A663, A666, A674, A675, A715, A724, |
| | A735 |

TABLE 10
KRAS WT FRET data

| IC50* | Examples |
|---|---|
| + | A388 |
| ++ | A075, A076, A076, A112, A132, A155, A167, A183, A194, |
| | A233, A256, A260, A261, A262, A263, A264, A265, A268, |
| | A270, A280, A294, A295, A296, A297, A298, A299, A300, |
| | A319, A333, A334, A338, A367, A382, A383, A385, A386, |
| | A387, A389, A418, A432, A433, A436, A464, A465, A479, |
| | A491, A492, A493, A527, A537, A542, A569, A594, A598, |
| | A602, A603, A605, A609, A621, A629, A633, A664, A665, |
| | A667, A679, A704, A711, A712, A734, A736, A737 |
| +++ | A005, A012, A016, A022, A028, A029, A037, A037, A043, |
| | A052, A054, A056, A069, A070, A070, A072, A074, A077, |
| | A082, A087, A088, A091, A093, A096, A097, A101, A105, |
| | A107, A110, A123, A128, A131, A141, A145, A147, A148, |
| | A149, A150, A153, A158, A164, A166, A180, A181, A182, |
| | A184, A189, A207, A244, A245, A246, A247, A250, A251, |
| | A252, A253, A254, A255, A257, A258, A259, A266, A267, |
| | A269, A275, A281, A281, A292, A305, A306, A334, A336, |
| | A337, A349, A350, A351, A353, A354, A356, A357, A358, |
| | A360, A361, A368, A369, A379, A384, A393, A394, A395, |
| | A397, A406, A407, A408, A409, A410, A415, A416, A417, |
| | A419, A420, A421, A430, A431, A435, A437, A438, A452, |
| | A454, A455, A457, A463, A490, A501, A517, A518, A519, |
| | A529, A536, A538, A539, A541, A543, A544, A545, A547, |
| | A548, A550, A552, A553, A554, A555, A557, A558, A564, |
| | A570, A571, A572, A573, A575, A577, A578, A580, A581, |
| | A582, A583, A586, A591, A595, A600, A604, A606, A607, |
| | A608, A610, A611, A612, A618, A620, A622, A624, A625, |
| | A630, A631, A632, A634, A635, A636, A647, A650, A651, |
| | A653, A654, A655, A656, A659, A660, A661, A662, A668, |
| | A669, A670, A671, A672, A676, A677, A678, A680, A681, |
| | A682, A683, A684, A685, A686, A687, A688, A690, A695, |
| | A696, A697, A698, A699, A700, A701, A702, A709, A710, |
| | A713, A714, A717, A718, A719, A722, A728, A729, A730, |
| | A731, A732, A738, A739 |
| ++++ | A004, A006, A007, A008, A010, A013, A014, A015, A017, |
| | A018, A019, A020, A025, A027, A030, A031, A032, A033, |
| | A034, A035, A036, A038, A039, A040, A041, A044, A045, |
| | A047, A048, A051, A055, A057, A058, A061, A063, A064, |
| | A067, A068, A071, A073, A075, A079, A081, A081, A083, |
| | A086, A089, A092, A095, A098, A103, A104, A108, A111, |
| | A112, A115, A115, A116, A117, A120, A121, A124, A125, |
| | A127, A129, A131, A132, A133, A133, A134, A135, A137, |
| | A138, A140, A145, A146, A152, A154, A155, A159, A161, |
| | A162, A163, A167, A168, A169, A172, A175, A178, A179, |
| | A181, A182, A183, A187, A193, A194, A196, A197, A199, |
| | A204, A206, A209, A212, A213, A214, A214, A214, A214, |
| | A216, A217, A222, A225, A225, A225, A225, A227, A232, |
| | A237, A238, A241, A241, A247, A249, A250, A251, A258, |
| | A261, A277, A278, A282, A283, A290, A291, A302, A309, |
| | A310, A314, A317, A320, A321, A324, A326, A328, A330, |
| | A331, A332, A335, A339, A340, A341, A342, A343, A344, |
| | A347, A348, A352, A359, A363, A364, A365, A366, A370, |
| | A371, A372, A373, A375, A376, A378, A380, A381, A390, |
| | A391, A392, A396, A398, A399, A401, A402, A411, A412, |
| | A413, A422, A423, A424, A426, A427, A439, A440, A443, |
| | A444, A445, A446, A447, A449, A451, A456, A458, A459, |
| | A461, A462, A466, A468, A469, A470, A471, A472, A473, |
| | A474, A475, A476, A477, A481, A483, A485, A487, A488, |
| | A489, A494, A495, A497, A498, A499, A502, A503, A504, |
| | A505, A506, A509, A510, A511, A512, A513, A515, A520, |
| | A521, A522, A525, A526, A528, A530, A531, A532, A533, |
| | A534, A540, A546, A549, A551, A556, A559, A560, A561, |
| | A562, A563, A566, A567, A574, A576, A579, A585, A588, |
| | A589, A590, A593, A597, A601, A616, A619, A623, A626, |
| | A637, A638, A639, A640, A641, A642, A645, A648, A652, |
| | A658, A673, A689, A691, A692, A693, A694, A703, A705, |
| | A706, A707, A708, A716, A720, A723, A725, A726, A727, |
| | A735 |
| +++++ | A001, A002, A003, A004, A009, A011, A012, A015, A021, |
| | A023, A024, A026, A032, A036, A037, A038, A042, A046, |
| | A048, A049, A050, A053, A053, A059, A060, A062, A065, |
| | A066, A078, A078, A080, A084, A085, A090, A094, A099, |
| | A100, A102, A106, A109, A113, A114, A118, Al19, A120, |
| | A121, A122, A124, A126, A130, A133, A135, A136, A139, |
| | A142, A143, A144, A144, A148, A151, A156, A157, A160, |
| | A165, A170, A171, A173, A174, A176, A177, A184, A185, |
| | A186, A186, A187, A188, A190, A191, A191, A192, A192, |
| | A195, A196, A198, A200, A201, A202, A203, A205, A208, |
| | A209, A210, A210, A210, A210, A211, A211, A211, A211, |
| | A212, A213, A215, A215, A216, A217, A218, A218, A219, |
| | A219, A219, A219, A220, A220, A221, A221, A222, A223, |
| | A223, A224, A224, A226, A228, A229, A230, A231, A231, |
| | A231, A234, A235, A236, A239, A239, A240, A242, A243, |
| | A248, A271, A272, A273, A274, A276, A279, A279, A279, |
| | A283, A283, A284, A285, A286, A287, A288, A289, A293, |
| | A301, A303, A304, A304, A304, A304, A307, A308, A311, |
| | A312, A313, A316, A316, A316, A318, A318, A322, A323, |
| | A325, A327, A328, A329, A345, A346, A355, A362, A374, |
| | A377, A400, A403, A404, A405, A414, A425, A428, A429, |
| | A441, A442, A448, A450, A453, A460, A467, A478, A480, |
| | A482, A484, A486, A496, A500, A507, A508, A514, A516, |
| | A523, A524, A535, A565, A568, A584, A587, A592, A596, |
| | A599, A613, A614, A615, A617, A627, A628, A643, A644, |
| | A646, A649, A657, A663, A666, A674, A675, A715, A721, |
| | A724, A733 |

TABLE 11
KRAS G12C FRET data

| IC50* | Examples |
|---|---|
| + | A038, A075, A076, A088, A095, A096, A097, A107, A108, |
| | A112, A116, A120, A121, A129, A130, A131, A131, A132, |
| | A133, A133, A135, A140, A141, A145, A157, A162, A167, |
| | A175, A176, A182, A183, A184, A194, A196, A209, A244, |
| | A245, A246, A247, A247, A255, A256, A257, A266, A268, |
| | A270, A290, A291, A292, A294, A298, A299, A300, A316, |
| | A316, A316, A317, A322, A323, A324, A326, A333, A334, |
| | A337, A339, A343, A346, A348, A351, A365, A375, A376, |
| | A377, A379, A380, A381, A388, A391, A403, A407, A411, |
| | A414, A425, A427, A474, A475, A477, A479, A509, A527, |
| | A529, A534, A542, A543, A544, A549, A550, A551, A556, |
| | A577, A578, A579, A591, A599, A600, A601, A609, A620, |
| | A626, A627, A628, A638, A642, A647, A658, A660, A663, |
| | A667, A673, A684, A689, A691, A692, A699, A705, A707, |
| | A711, A721, A726, A727, A735 |
| ++ | A004, A005, A012, A015, A016, A020, A022, A027, A028, |
| | A029, A031, A032, A037, A037, A038, A043, A044, A047, |
| | A048, A051, A052, A054, A055, A056, A057, A058, A064, |
| | A067, A069, A070, A070, A071, A072, A074, A076, A077, |
| | A079, A082, A083, A086, A087, A091, A093, A103, A104, |
| | A105, A115, A121, A123, A124, A126, A127, A128, A132, |
| | A133, A134, A135, A138, A139, A142, A144, A148, A149, |
| | A150, A152, A153, A154, A155, A155, A156, A158, A164, |
| | A165, A166, A172, A178, A179, A181, A182, A183, A186, |
| | A187, A189, A191, A193, A196, A200, A201, A204, A207, |
| | A209, A214, A214, A225, A225, A233, A237, A238, A241, |
| | A241, A250, A250, A251, A251, A252, A253, A260, A261, |
| | A262, A263, A264, A265, A267, A269, A275, A280, A281, |
| | A283, A295, A296, A297, A305, A319, A328, A332, A334, |
| | A336, A338, A342, A344, A349, A350, A352, A353, A359, |
| | A361, A362, A363, A366, A367, A368, A370, A371, A382, |
| | A383, A384, A385, A386, A387, A389, A394, A396, A397, |
| | A400, A410, A416, A417, A418, A421, A423, A432, A433, |
| | A435, A436, A437, A438, A439, A440, A443, A444, A445, |
| | A448, A452, A454, A456, A458, A459, A463, A464, A465, |
| | A466, A476, A478, A485, A489, A490, A491, A492, A493, |
| | A499, A501, A503, A504, A505, A510, A513, A515, A517, |
| | A518, A519, A525, A526, A528, A532, A533, A536, A537, |
| | A538, A545, A552, A558, A559, A560, A566, A567, A568, |
| | A569, A572, A573, A575, A580, A581, A584, A585, A587, |
| | A589, A590, A592, A594, A595, A596, A597, A598, A602, |
| | A603, A610, A612, A614, A615, A616, A617, A621, A622, |
| | A624, A629, A632, A634, A636, A637, A643, A644, A645, |
| | A646, A649, A650, A651, A653, A657, A659, A661, A662, |
| | A664, A665, A668, A669, A671, A672, A674, A675, A678, |
| | A679, A681, A682, A683, A685, A686, A697, A698, A700, |
| | A701, A703, A704, A706, A709, A710, A712, A715, A719, |
| | A720, A722, A724, A730, A733, A734, A736, A737 |
| +++ | A001, A003, A006, A007, A008, A010, A011, A013, A014, |
| | A017, A018, A019, A023, A025, A026, A030, A033, A034, |
| | A035, A039, A040, A041, A042, A045, A046, A050, A053, |
| | A053, A060, A061, A062, A063, A065, A066, A068, A073, |
| | A075, A078, A078, A080, A081, A081, A089, A092, A094, |
| | A098, A099, A101, A109, A110, A111, A112, A113, A115, |
| | A117, A118, A119, A120, A125, A137, A143, A144, A145, |
| | A146, A147, A151, A159, A160, A161, A167, A168, A169, |
| | A173, A174, A177, A180, A181, A184, A185, A188, A190, |
| | A191, A192, A194, A195, A197, A199, A205, A206, A208, |
| | A210, A211, A212, A213, A215, A216, A217, A218, A220, |
| | A221, A222, A223, A224, A226, A227, A229, A232, A249, |
| | A254, A258, A258, A259, A261, A277, A278, A281, A282, |
| | A284, A301, A302, A306, A310, A314, A320, A321, A327, |
| | A330, A331, A335, A340, A341, A347, A354, A356, A357, |
| | A358, A360, A364, A369, A372, A373, A374, A378, A390, |
| | A392, A393, A395, A399, A402, A404, A406, A408, A409, |
| | A412, A413, A415, A419, A420, A422, A424, A426, A430, |
| | A431, A442, A446, A447, A449, A451, A455, A457, A461, |
| | A462, A468, A469, A470, A471, A472, A473, A481, A483, |
| | A484, A486, A487, A488, A494, A495, A497, A498, A502, |
| | A506, A507, A508, A511, A512, A520, A522, A524, A530, |
| | A531, A539, A540, A541, A546, A547, A548, A553, A554, |
| | A555, A557, A561, A562, A563, A564, A565, A570, A571, |
| | A574, A576, A582, A583, A586, A588, A593, A604, A605, |
| | A606, A607, A608, A611, A613, A618, A619, A623, A625, |
| | A630, A631, A633, A635, A639, A640, A641, A648, A652, |
| | A654, A655, A656, A670, A676, A677, A680, A687, A688, |
| | A690, A693, A694, A695, A696, A702, A708, A713, A714, |
| | A716, A717, A718, A723, A725, A728, A729, A731, A732, |
| | A738, A739 |
| ++++ | A002, A004, A009, A012, A015, A021, A024, A032, A036, |
| | A037, A048, A049, A059, A084, A085, A090, A100, A102, |
| | A106, A124, A136, A148, A163, A170, A171, A186, A187, |
| | A192, A202, A203, A210, A211, A212, A214, A214, A216, |
| | A218, A219, A219, A220, A222, A224, A225, A225, A228, |
| | A235, A240, A242, A248, A272, A274, A276, A279, A283, |
| | A283, A289, A303, A307, A309, A313, A325, A328, A345, |
| | A398, A401, A405, A441, A453, A460, A467, A480, A496, |
| | A514, A516, A521, A523, A535, A666 |
| +++++ | A036, A114, A122, A198, A210, A210, A211, A211, A213, |
| | A215, A217, A219, A219, A221, A223, A230, A231, A231, |
| | A231, A234, A236, A239, A239, A243, A271, A273, A279, |
| | A279, A285, A286, A287, A288, A293, A304, A304, A304, |
| | A304, A308, A311, A312, A318, A318, A329, A355, A428, |
| | A429, A450, A482, A500 |

TABLE 12
KRAS G13D FRET data

| IC50* | Examples |
|---|---|
| + | None |
| ++ | A075, A076, A112, A155, A183, A260, A261, A262, A263, |
| | A264, A265, A267, A268, A270, A294, A296, A298, A300, |
| | A319, A333, A338, A388, A464, A465, A527, A537, A542, |
| | A664 |
| +++ | A028, A054, A096, A105, A107, A123, A128, A131, A132, |
| | A141, A145, A149, A150, A153, A158, A164, A167, A181, |
| | A182, A184, A189, A194, A196, A207, A233, A244, A245, |
| | A246, A247, A250, A251, A252, A253, A255, A256, A257, |
| | A258, A261, A266, A269, A275, A280, A281, A281, A292, |
| | A295, A297, A299, A305, A306, A334, A334, A336, A337, |
| | A349, A350, A353, A354, A357, A361, A367, A368, A379, |
| | A382, A383, A384, A385, A386, A387, A389, A394, A395, |
| | A397, A406, A407, A410, A415, A416, A417, A418, A421, |
| | A432, A433, A436, A438, A463, A479, A490, A491, A492, |
| | A493, A518, A519, A529, A536, A538, A543, A544, A545, |
| | A555, A558, A573, A577, A578, A580, A581, A582, A591, |
| | A594, A596, A598, A602, A603, A605, A606, A608, A609, |
| | A611, A612, A620, A621, A622, A624, A629, A631, A633, |
| | A650, A651, A653, A654, A655, A659, A661, A662, A665, |
| | A667, A669, A670, A671, A672, A678, A679, A681, A682, |
| | A683, A684, A685, A686, A687, A690, A695, A697, A698, |
| | A699, A700, A701, A704, A709, A710, A711, A712, A718, |
| | A719, A729, A730, A734, A736, A737 |
| ++++ | A016, A038, A069, A075, A088, A095, A103, A104, A108, |
| | A110, A111, A112, A116, A117, A120, A120, A121, A121, |
| | A125, A127, A129, A130, A131, A132, A133, A133, A133, |
| | A134, A135, A138, A140, A142, A144, A144, A145, A146, |
| | A147, A148, A152, A154, A155, A156, A157, A159, A161, |
| | A162, A163, A166, A175, A178, A179, A180, A181, A182, |
| | A183, A187, A193, A194, A197, A206, A209, A212, A213, |
| | A232, A237, A238, A241, A241, A247, A249, A251, A254, |
| | A258, A259, A277, A278, A282, A290, A302, A314, A321, |
| | A326, A328, A330, A331, A332, A335, A339, A340, A341, |
| | A342, A344, A351, A356, A358, A359, A360, A363, A364, |
| | A365, A366, A369, A370, A372, A373, A380, A381, A390, |
| | A391, A393, A396, A399, A401, A408, A409, A411, A412, |
| | A413, A419, A420, A422, A424, A427, A430, A431, A435, |
| | A437, A439, A440, A443, A444, A445, A446, A449, A451, |
| | A452, A454, A455, A456, A457, A458, A459, A466, A468, |
| | A469, A470, A472, A473, A474, A475, A476, A485, A487, |
| | A488, A489, A499, A501, A502, A503, A504, A505, A509, |
| | A510, A511, A513, A515, A517, A520, A521, A522, A525, |
| | A534, A539, A540, A541, A546, A547, A548, A549, A550, |
| | A551, A552, A553, A554, A557, A559, A560, A561, A562, |
| | A563, A564, A566, A567, A570, A571, A572, A574, A575, |
| | A583, A586, A588, A593, A595, A597, A600, A601, A604, |
| | A607, A610, A616, A618, A619, A623, A625, A626, A630, |
| | A632, A634, A635, A636, A637, A638, A640, A641, A642, |
| | A645, A647, A652, A656, A658, A660, A668, A673, A676, |
| | A677, A680, A688, A689, A691, A693, A694, A696, A702, |
| | A706, A707, A713, A714, A717, A720, A722, A725, A726, |
| | A727, A728, A731, A732, A738, A739 |
| +++++ | A024, A065, A094, A102, A106, A109, A113, A114, A115, |
| | A115, A118, A119, A122, A124, A124, A126, A135, A136, |
| | A137, A139, A143, A148, A151, A160, A165, A167, A168, |
| | A169, A170, A171, A172, A173, A174, A176, A177, A184, |
| | A185, A186, A186, A187, A188, A190, A191, A191, A192, |
| | A192, A195, A196, A198, A199, A200, A201, A202, A203, |
| | A204, A205, A208, A209, A210, A210, A210, A210, A211, |
| | A211, A211, A211, A212, A213, A214, A214, A214, A225, |
| | A225, A225, A226, A227, A228, A229, A230, A231, A231, |
| | A231, A234, A235, A236, A239, A239, A240, A242, A243, |
| | A248, A250, A271, A272, A273, A274, A276, A279, A279, |
| | A279, A283, A283, A283, A284, A285, A286, A287, A288, |
| | A289, A291, A293, A301, A303, A304, A304, A304, A304, |
| | A307, A308, A309, A310, A311, A312, A313, A316, A316, |
| | A316, A317, A318, A318, A320, A322, A323, A324, A325, |
| | A327, A328, A329, A343, A345, A346, A347, A348, A352, |
| | A355, A362, A371, A374, A375, A376, A377, A378, A392, |
| | A398, A400, A402, A403, A404, A405, A414, A423, A425, |
| | A426, A428, A429, A441, A442, A447, A448, A450, A453, |
| | A460, A461, A462, A467, A471, A477, A478, A480, A481, |
| | A482, A483, A484, A486, A494, A495, A496, A497, A498, |
| | A500, A506, A507, A508, A512, A514, A516, A523, A524, |
| | A526, A528, A530, A531, A532, A533, A535, A556, A565, |
| | A568, A569, A576, A579, A584, A585, A587, A589, A590, |
| | A592, A599, A613, A614, A615, A617, A627, A628, A639, |
| | A643, A644, A646, A648, A649, A657, A663, A666, A674, |
| | A675, A692, A703, A705, A708, A715, A716, A721, A723, |
| | A724, A733, A735 |

TABLE 13
KRAS Q61H FRET data

| IC50* | Examples |
|---|---|
| + | A297, A299, A388, A598, A621 |
| ++ | A012, A022, A075, A076, A077, A082, A087, A105, A148, |
| | A167, A181, A207, A262, A275, A281, A300, A333, A338, |
| | A349, A350, A353, A354, A367, A368, A379, A382, A383, |
| | A384, A385, A386, A387, A389, A394, A407, A416, A417, |
| | A463, A491, A492, A493, A537, A542, A543, A544, A545, |
| | A557, A577, A578, A580, A582, A586, A594, A596, A602, |
| | A603, A605, A606, A608, A609, A611, A612, A620, A622, |
| | A629, A631, A633, A650, A651, A653, A654, A655, A656, |
| | A661, A662, A664, A665, A667, A668, A669, A671, A672, |
| | A676, A678, A679, A681, A682, A683, A684, A686, A687, |
| | A696, A697, A698, A701, A702, A704, A709, A710, A711, |
| | A712, A718, A719, A734, A736, A737, A739 |
| +++ | A016, A045, A064, A074, A083, A086, A089, A104, A115, |
| | A166, A172, A193, A252, A277, A314, A328, A330, A332, |
| | A340, A341, A342, A344, A351, A356, A357, A358, A360, |
| | A361, A365, A366, A369, A372, A373, A390, A393, A468, |
| | A474, A475, A494, A502, A503, A504, A505, A522, A534, |
| | A538, A539, A540, A541, A546, A547, A548, A549, A551, |
| | A552, A553, A554, A555, A558, A559, A560, A561, A564, |
| | A566, A567, A570, A571, A572, A573, A574, A575, A581, |
| | A583, A588, A591, A593, A595, A597, A600, A604, A607, |
| | A610, A618, A619, A623, A624, A625, A626, A630, A632, |
| | A634, A635, A636, A640, A659, A660, A670, A673, A677, |
| | A680, A685, A688, A689, A690, A694, A695, A699, A700, |
| | A706, A713, A714, A717, A725, A728, A729, A730, A731, |
| | A732, A738 |
| ++++ | A023, A025, A026, A050, A053, A060, A061, A062, A080, |
| | A084, A085, A102, A113, A136, A143, A151, A160, A168, |
| | A202, A210, A211, A226, A227, A242, A278, A290, A301, |
| | A302, A303, A331, A335, A339, A343, A345, A346, A347, |
| | A348, A352, A359, A362, A363, A364, A370, A371, A376, |
| | A380, A381, A391, A392, A403, A427, A442, A447, A461, |
| | A472, A481, A495, A496, A498, A531, A550, A562, A563, |
| | A565, A568, A569, A576, A579, A584, A585, A589, A590, |
| | A592, A601, A616, A627, A637, A638, A639, A641, A642, |
| | A645, A647, A652, A658, A666, A674, A691, A692, A693, |
| | A703, A705, A707, A708, A716, A720, A721, A722, A723, |
| | A726, A727, A733, A735 |
| +++++ | A170, A171, A205, A240, A243, A248, A285, A304, A304, |
| | A313, A316, A329, A355, A374, A375, A377, A378, A482, |
| | A486, A507, A587, A599, A613, A614, A615, A617, A628, |
| | A643, A644, A646, A648, A649, A657, A663, A675, A715, |
| | A724 |

TABLE 14
NRAS G12C FRET data

| IC50* | Examples |
|---|---|
| + | A038, A075, A076, A088, A095, A096, A107, A108, A112, A116, A120, A121, |
| | A123, A129, A130, A131, A131, A132, A133, A133, A135, A140, A141, A145, |
| | A155, A157, A162, A167, A175, A176, A182, A183, A184, A194, A196, A209, |
| | A244, A245, A246, A247, A247, A250, A255, A257, A266, A268, A270, A290, |
| | A292, A294, A297, A298, A299, A300, A316, A316, A316, A323, A324, A326, |
| | A333, A334, A336, A337, A339, A343, A348, A351, A365, A375, A376, A377, |
| | A380, A388, A391, A407, A411, A414, A427, A435, A474, A475, A479, A509, |
| | A527, A529, A534, A537, A542, A543, A544, A550, A551, A556, A577, A578, |
| | A579, A591, A600, A601, A609, A612, A616, A620, A626, A627, A628, A638, |
| | A642, A647, A658, A660, A663, A667, A673, A684, A689, A691, A692, A699, |
| | A705, A707, A711, A721, A727, A735 |
| ++ | A016, A054, A069, A103, A104, A105, A115, A121, A124, A126, A127, A128, |
| | A132, A133, A134, A135, A138, A142, A144, A148, A149, A150, A152, A153, |
| | A154, A155, A158, A164, A165, A166, A172, A178, A179, A181, A182, A183, |
| | A186, A187, A189, A191, A196, A200, A204, A207, A233, A237, A241, A241, |
| | A250, A251, A251, A252, A256, A260, A261, A262, A263, A264, A265, A267, |
| | A269, A275, A281, A291, A295, A296, A305, A317, A319, A322, A328, A332, |
| | A334, A338, A344, A346, A349, A350, A352, A353, A354, A361, A362, A363, |
| | A367, A368, A370, A371, A379, A381, A382, A383, A384, A385, A386, A387, |
| | A389, A394, A396, A397, A400, A403, A410, A417, A418, A419, A421, A423, |
| | A425, A432, A433, A436, A437, A438, A439, A440, A443, A444, A445, A448, |
| | A452, A454, A456, A458, A459, A463, A464, A465, A466, A476, A477, A478, |
| | A485, A489, A490, A491, A492, A493, A499, A501, A503, A504, A505, A510, |
| | A513, A515, A517, A518, A519, A525, A526, A528, A532, A533, A536, A538, |
| | A545, A549, A552, A558, A559, A560, A566, A567, A569, A572, A573, A574, |
| | A575, A580, A581, A582, A584, A585, A587, A589, A590, A592, A594, A595, |
| | A596, A597, A598, A599, A602, A603, A610, A614, A615, A617, A621, A622, |
| | A624, A629, A631, A632, A633, A634, A636, A637, A643, A644, A645, A646, |
| | A649, A650, A651, A653, A654, A657, A659, A661, A662, A664, A665, A668, |
| | A669, A671, A672, A674, A675, A676, A678, A679, A681, A682, A683, A685, |
| | A686, A695, A697, A698, A700, A701, A703, A704, A706, A709, A710, A712, |
| | A715, A719, A720, A722, A724, A726, A730, A733, A734, A736, A737 |
| +++ | A065, A075, A094, A109, A111, A112, A113, A115, A117, A118, A119, A120, |
| | A137, A139, A143, A144, A145, A146, A147, A151, A156, A159, A161, A167, |
| | A169, A173, A177, A180, A181, A184, A185, A188, A190, A192, A193, A194, |
| | A195, A197, A199, A201, A206, A209, A210, A211, A212, A214, A225, A226, |
| | A227, A232, A238, A249, A253, A254, A258, A258, A259, A261, A277, A278, |
| | A280, A281, A282, A283, A301, A302, A306, A310, A314, A320, A321, A327, |
| | A330, A331, A335, A340, A341, A342, A347, A356, A357, A358, A359, A360, |
| | A364, A366, A369, A372, A373, A374, A378, A390, A392, A393, A395, A399, |
| | A402, A404, A406, A408, A409, A412, A413, A415, A416, A420, A422, A424, |
| | A430, A431, A442, A446, A447, A449, A451, A455, A457, A461, A462, A467, |
| | A468, A469, A470, A471, A472, A473, A481, A483, A486, A487, A488, A494, |
| | A495, A497, A498, A502, A506, A507, A508, A511, A512, A520, A522, A524, |
| | A530, A531, A539, A540, A541, A546, A547, A548, A553, A554, A555, A557, |
| | A561, A562, A563, A564, A565, A568, A570, A571, A576, A583, A586, A588, |
| | A593, A604, A605, A606, A607, A608, A611, A618, A619, A623, A625, A630, |
| | A635, A639, A640, A641, A648, A652, A655, A656, A670, A677, A680, A687, |
| | A688, A690, A693, A694, A696, A702, A708, A713, A714, A716, A717, A718, |
| | A723, A725, A728, A729, A731, A732, A738, A739 |
| ++++ | A024, A102, A106, A110, A124, A125, A136, A160, A163, A168, A170, A171, |
| | A174, A186, A187, A191, A192, A202, A203, A205, A208, A210, A211, A212, |
| | A213, A228, A229, A235, A240, A242, A248, A274, A276, A279, A283, A284, |
| | A289, A303, A307, A309, A313, A325, A328, A345, A398, A401, A426, A441, |
| | A453, A460, A480, A484, A496, A514, A516, A521, A523, A535, A613, A666 |
| +++++ | A114, A122, A148, A198, A210, A210, A211, A211, A213, A214, A214, A225, |
| | A225, A230, A231, A231, A231, A234, A236, A239, A239, A243, A271, A272, |
| | A273, A279, A279, A283, A285, A286, A287, A288, A293, A304, A304, A304, |
| | A304, A308, A311, A312, A318, A318, A329, A355, A405, A428, A429, A450, |
| | A482, A500 |

TABLE 15
NRAS Q61R FRET data

| IC50* | Examples |
|---|---|
| + | A388 |
| ++ | A367, A368, A382, A383, A385, A386, A387, A389, A407, A417, A491, A492, |
| | A493, A542, A577, A594, A596, A598, A602, A603, A609, A611, A612, A621, |
| | A629, A633, A655, A664, A665, A667, A669, A678, A679, A697, A704, A712, |
| | A719, A734, A736, A737 |
| +++ | A012, A022, A075, A076, A082, A087, A105, A148, A167, A181, A207, A262, |
| | A275, A281, A297, A299, A300, A314, A333, A338, A349, A350, A353, A354, |
| | A356, A357, A360, A361, A369, A379, A384, A393, A394, A416, A463, A502, |
| | A537, A538, A539, A541, A543, A544, A545, A546, A548, A554, A555, A570, |
| | A571, A572, A573, A575, A578, A580, A581, A582, A583, A586, A591, A593, |
| | A595, A597, A604, A605, A606, A607, A608, A610, A618, A619, A620, A622, |
| | A624, A625, A630, A631, A632, A634, A635, A636, A650, A651, A653, A654, |
| | A656, A659, A661, A662, A668, A670, A671, A672, A676, A677, A680, A681, |
| | A682, A683, A684, A685, A686, A687, A688, A690, A695, A696, A698, A699, |
| | A700, A701, A702, A706, A709, A710, A711, A713, A714, A717, A718, A728, |
| | A732, A738 |
| ++++ | A016, A064, A074, A077, A083, A086, A089, A104, A115, A166, A168, A193, |
| | A252, A277, A290, A328, A330, A332, A335, A339, A340, A341, A342, A343, |
| | A344, A347, A351, A352, A358, A359, A363, A364, A365, A366, A370, A372, |
| | A373, A380, A381, A390, A391, A392, A442, A461, A472, A474, A475, A481, |
| | A494, A495, A496, A498, A503, A504, A505, A522, A531, A534, A540, A547, |
| | A549, A550, A551, A552, A553, A557, A558, A559, A560, A561, A564, A566, |
| | A567, A574, A588, A600, A601, A623, A626, A638, A639, A640, A641, A647, |
| | A652, A658, A660, A673, A689, A691, A693, A694, A720, A722, A725, A727, |
| | A729, A730, A731, A739 |
| +++++ | A023, A025, A026, A045, A050, A053, A060, A061, A062, A080, A084, A085, |
| | A102, A113, A136, A143, A151, A160, A170, A171, A172, A202, A205, A210, |
| | A211, A226, A227, A240, A242, A243, A248, A278, A285, A301, A302, A303, |
| | A304, A304, A313, A316, A329, A331, A345, A346, A348, A355, A362, A371, |
| | A374, A375, A376, A377, A378, A403, A427, A447, A468, A482, A486, A507, |
| | A556, A562, A563, A565, A568, A569, A576, A579, A584, A585, A587, A589, |
| | A590, A592, A599, A613, A614, A615, A616, A617, A627, A628, A637, A642, |
| | A643, A644, A645, A646, A648, A649, A657, A663, A666, A674, A675, A692, |
| | A703, A705, A707, A708, A715, A716, A721, A723, A724, A726, A733, A735 |

TABLE 16
NRAS Q61K FRET data

| IC50* | Examples |
|---|---|
| + | A388 |
| ++ | A262, A297, A299, A338, A349, A350, A353, A354, A367, A368, A369, A382, |
| | A383, A384, A385, A386, A387, A389, A394, A407, A416, A417, A463, A491, |
| | A492, A493, A542, A543, A544, A545, A577, A580, A582, A594, A596, A598, |
| | A602, A603, A609, A611, A612, A620, A621, A622, A629, A631, A633, A650, |
| | A651, A653, A654, A655, A661, A662, A664, A665, A667, A668, A669, A671, |
| | A678, A679, A681, A682, A683, A686, A687, A696, A697, A698, A701, A702, |
| | A704, A709, A710, A711, A712, A718, A719, A734, A736, A737, A739 |
| +++ | A012, A022, A074, A075, A076, A077, A082, A086, A087, A105, A148, A167, |
| | A181, A207, A275, A277, A281, A300, A314, A330, A333, A340, A356, A360, |
| | A361, A379, A390, A393, A502, A537, A538, A539, A541, A546, A547, A548, |
| | A554, A555, A561, A570, A571, A572, A573, A574, A575, A578, A581, A583, |
| | A586, A588, A593, A595, A597, A604, A605, A606, A607, A608, A610, A618, |
| | A619, A623, A624, A625, A630, A632, A634, A635, A636, A656, A660, A670, |
| | A672, A676, A677, A680, A684, A685, A688, A690, A694, A695, A699, A700, |
| | A706, A713, A714, A717, A728, A729, A730, A731, A732, A738 |
| ++++ | A016, A025, A045, A050, A060, A061, A064, A080, A083, A089, A104, A115, |
| | A143, A151, A166, A168, A172, A193, A202, A210, A211, A227, A252, A278, |
| | A290, A328, A331, A332, A335, A339, A341, A342, A343, A344, A347, A351, |
| | A352, A357, A358, A359, A363, A364, A365, A366, A370, A372, A373, A380, |
| | A381, A391, A427, A442, A461, A468, A472, A474, A475, A481, A494, A495, |
| | A496, A498, A503, A504, A505, A522, A531, A534, A540, A549, A550, A551, |
| | A552, A553, A557, A558, A559, A560, A562, A563, A564, A566, A567, A576, |
| | A590, A591, A600, A601, A626, A637, A638, A639, A640, A641, A647, A652, |
| | A659, A673, A689, A691, A693, A705, A720, A722, A725, A727, A735 |
| +++++ | A023, A026, A053, A062, A084, A085, A102, A113, A136, A160, A170, A171, |
| | A205, A226, A240, A242, A243, A248, A285, A301, A302, A303, A304, A304, |
| | A313, A316, A329, A345, A346, A348, A355, A362, A371, A374, A375, A376, |
| | A377, A378, A392, A403, A447, A482, A486, A507, A556, A565, A568, A569, |
| | A579, A584, A585, A587, A589, A592, A599, A613, A614, A615, A616, A617, |
| | A627, A628, A642, A643, A644, A645, A646, A648, A649, A657, A658, A663, |
| | A666, A674, A675, A692, A703, A707, A708, A715, A716, A721, A723, A724, |
| | A726, A733 |

TABLE 17
NBAS WT FRET data

| IC50* | Examples |
|---|---|
| + | A388 |
| ++ | A075, A076, A087, A167, A262, A275, A297, A299, A300, A333, A338, A349, |
| | A367, A368, A382, A383, A385, A386, A387, A389, A407, A417, A491, A492, |
| | A493, A537, A542, A577, A594, A596, A598, A602, A603, A605, A609, A612, |
| | A621, A629, A633, A664, A665, A667, A669, A679, A697, A704, A709, A711, |
| | A712, A719, A734, A736, A737 |
| +++ | A012, A016, A022, A074, A077, A082, A086, A105, A148, A166, A181, A207, |
| | A252, A277, A281, A340, A350, A351, A353, A354, A356, A357, A358, A360, |
| | A361, A369, A379, A384, A393, A394, A416, A463, A502, A538, A539, A541, |
| | A543, A544, A545, A546, A547, A548, A550, A552, A554, A555, A557, A558, |
| | A564, A570, A571, A572, A573, A575, A578, A580, A581, A582, A583, A586, |
| | A591, A593, A595, A600, A604, A606, A607, A608, A610, A611, A618, A620, |
| | A622, A624, A625, A630, A631, A632, A634, A635, A636, A647, A650, A651, |
| | A653, A654, A655, A656, A659, A660, A661, A662, A668, A670, A671, A672, |
| | A676, A677, A678, A680, A681, A682, A683, A684, A685, A686, A687, A688, |
| | A690, A695, A696, A698, A699, A700, A701, A702, A710, A713, A714, A717, |
| | A718, A722, A728, A729, A730, A731, A732, A738, A739 |
| ++++ | A025, A045, A060, A061, A062, A064, A083, A089, A104, A115, A151, A168, |
| | A172, A193, A202, A210, A211, A227, A278, A290, A314, A328, A330, A331, |
| | A332, A335, A339, A341, A342, A343, A344, A347, A348, A352, A359, A362, |
| | A363, A364, A365, A366, A370, A371, A372, A373, A375, A376, A378, A380, |
| | A381, A390, A391, A392, A427, A447, A461, A468, A472, A474, A475, A481, |
| | A494, A495, A496, A498, A503, A504, A505, A507, A522, A531, A534, A540, |
| | A549, A551, A553, A556, A559, A560, A561, A562, A563, A566, A567, A574, |
| | A576, A579, A584, A585, A588, A589, A590, A597, A601, A616, A619, A623, |
| | A626, A637, A638, A639, A640, A641, A642, A645, A648, A652, A658, A673, |
| | A689, A691, A693, A694, A703, A705, A706, A707, A708, A716, A720, A721, |
| | A723, A725, A726, A727, A735 |
| +++++ | A023, A026, A050, A053, A080, A084, A085, A102, A113, A136, A143, A160, |
| | A170, A171, A205, A226, A240, A242, A243, A248, A285, A302, A303, A304, |
| | A304, A313, A316, A329, A345, A346, A355, A374, A377, A403, A442, A482, |
| | A486, A565, A568, A569, A587, A592, A599, A613, A614, A615, A617, A627, |
| | A628, A643, A644, A646, A649, A657, A663, A666, A674, A675, A692, A715, |
| | A724, A733 |

### In Vitro Cell Proliferation Panels

Potency for inhibition of cell growth was assessed at CrownBio using standard methods. Briefly, cell lines were cultured in appropriate medium, and then plated in 3D methylcellulose. Inhibition of cell growth was determined by CellTiter-Glo® after 5 days of culture with increasing concentrations of compounds. Compound potency was reported as the 50% inhibition concentration (absolute IC50). The assay took place over 7 days. On day 1, cells in 2D culture were harvested during logarithmic growth and suspended in culture medium at 1×105 cells/mi. Higher or lower cell densities were used for some cell lines based on prior optimization. 3.5 ml of cell suspension was mixed with 6.5% growth medium with 1% methylcellulose, resulting in a cell suspension in 0.65% methylcellulose. 90 μl of this suspension was distributed in the wells of 2 96-well plates. One plate was used for day 0 reading and 1 plate was used for the end-point experiment. Plates were incubated overnight at 37 C with 5% CO2. On day 2, one plate (for t reading) was removed and 10 μl growth medium plus 100 s CellTiter-Glo® Reagent was added to each well. After mixing and a 10 minute incubation, luminescence was recorded on an EnVision Multi-Label Reader (Perkin Elmer). Compounds in DMSO were diluted in growth medium such that the final, maximum concentration of compound was 10 μM, and serial 4-fold dilutions were performed to generate a 9-point concentration series. 10 μl of compound solution at 10 times final concentration was added to wells of the second plate. Plate was then incubated for 120 hours at 37 C and 5% CO2. On day 7 the plates were removed, 100 μl CellTiter-Glo® Reagent was added to each well, and after mixing and a 10 minute incubation, luminescence was recorded on an EnVision Multi-Label Reader (Perkin Elmer). Data was exported to GeneData Screener and modeled with a sigmoidal concentration response model in order to determine the IC50 for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite 8 having a KRAS G2C mutation, is insensitive to the KRAS G12C (OFF) inhibitor MRTX-849 (Hallin et al., Cancer Discovery 10:54-71 (2020)), and the cell line SW1573, which is insensitive to the KRAS G12C (OFF) inhibitor AMG51n (Canon et al., Nature 575:217-223 (2019)).

TABLE 18
IC50 values for various cancer cell lines with Compound B

| Cell Line | Histotype | Mutant | IC50* |
|---|---|---|---|
| NCI-H358 | Lung | KRAS G12C | very sensitive |
| MIA PaCa-2 | Pancreas | KRAS G12C | very sensitive |
| SW837 | Intestine/Large/Colorectum | KRAS G12C | very sensitive |
| KYSE-410 | HN/Esophapus | KRAS G12C | moderately sensitive |
| NCI-H727 | Lung | KRAS G12V | moderately sensitive |
| OVCAR-5 | Ovary | KRAS G12V | moderately sensitive |
| Capan-2 | Pancreas | KRAS G12V | moderately sensitive |
| NCI-H747 | Intestine/Large/Colorectum | other KRAS (G13D) | moderately sensitive |
| NCI-H441 | Lung | KRAS G12V | moderately sensitive |
| HEC-1-A | Uterus | KRAS G12D | moderately sensitive |
| NOZ | Liver/Bile duct | KRAS G12V | moderately sensitive |
| HCT116 | Intestine/Large/Colorectum | other KRAS (G13D) | moderately sensitive |
| Calu-6 | Lung | other KRAS (Q61K) | moderately sensitive |
| HuCCT1 | Liver/Bile duct | KRAS G12D | moderately sensitive |
| NCI-H2009 | Lung | other KRAS (G12A) | low sensitivity |
| NCI-H1975 | Lung | other MAPK (EGFR | low sensitivity |
| | | T790M, L858R) | |
| SW1573 | Lung | KRAS G12C | not sensitive |
| AGS | Stomach | KRAS G12D | low sensitivity |
| AsPC-1 | Pancreas | KRAS G12D | low sensitivity |
| SNU-668 | Stomach | other KRAS (Q61K) | low sensitivity |
| HPAC | Pancreas | KRAS G12D | low sensitivity |
| NCI-H1838 | Lung | other MAPK (NF1 mut) | low sensitivity |
| NCI-H3122 | Lung | other MAPK | low sensitivity |
| | | (EML4-ALK(E13, A20)) | |
| NCI-H460 | Lung | other KRAS (Q61H) | low sensitivity |
| SW403 | Intestine/Large/Colorectum | KRAS G12V | low sensitivity |
| A549 | Lung | other KRAS (G12S) | low sensitivity |
| CAL-62 | HN/Thyroid | other KRAS (G12R) | low sensitivity |
| DV-90 | Lung | other KRAS (G13D) | low sensitivity |
| OZ | Liver/Bile duct | other KRAS (Q61L) | low sensitivity |
| A-375 | Skin | BRAF V600E | low sensitivity |
| BxPC-3 | Pancreas | other MAPK (BRAF | low sensitivity |
| | | V487_P492delinsA) | |
| SW48 | Intestine/Large/Colorectum | not MAPK (PIK3CA | low sensitivity |
| | | G914R, EGFR G719S) | |
| HCC1588 | Lung | KRAS G12D | low sensitivity |
| TOV-21G | Ovary | other KRAS (G13C) | low sensitivity |
| SW948 | Intestine/Large/Colorectum | other KRAS (Q61L) | low sensitivity |
| LS513 | Intestine/Large/Colorectum | KRAS G12D | low sensitivity |
| MeWo | Skin | other MAPK (NF1 mut) | low sensitivity |

*Key:
low sensitivity: IC50 ≥ 1 uM
moderately sensitive: 1 uM > IC50 ≥ 0.1 uM
very sensitive: IC50 < 0.1 uM

TABLE 19
Summary of IC50 results for various cancer cell lines with several
compounds of the present invention (Compounds B and E-M)

| Cell Line | Histotype | Mutant | E | F | G | H | I | J | K | L | B | M |
|---|---|---|---|---|---|---|---|---|---|---|---|---|
| SW837 | Intestine/Large/ | KRAS G12C | V | V | V | V | V | V | V | V | V | V |
| | Colorectum | | | | | | | | | | | |
| MIA PaCa-2 | Pancreas | KRAS G12C | V | V | V | V | V | V | V | V | V | V |
| KYSE-410 | HN/Esophagus | KRAS G12C | L | L | L | L | L | L | L | L | L | L |
| H358 | Lung | KRAS G12C | V | V | V | V | V | V | V | V | V | V |
| H2122 | Lung | KRAS G12C | V | V | V | V | V | V | V | V | V | V |
| H1373 | Lung | KRAS G12C | V | V | V | V | V | V | V | V | V | V |
| H23 | Lung | KRAS G12C | V | V | V | V | V | V | V | V | V | V |
| H1792 | Lung | KRAS G12C | V | V | V | V | V | V | V | V | V | V |
| AsPC-1 | Pancreas | KRAS G12D | L | L | M | L | L | M | L | L | M | L |
| A375 | Skin | BRAF V600E | L | L | L | L | L | L | L | L | L | L |
| H1975 | Lung | other MAPK | M | L | M | L | L | M | L | M | M | L |
| | | (EGFR T790M, | | | | | | | | | | |
| | | L858R) | | | | | | | | | | |
| HCC1588 | Lung | KRAS G12D | L | L | L | L | L | L | L | L | L | L |
| H441 | Lung | KRAS G12V | M | M | M | L | M | V | L | M | V | L |

*Key:
(L) low sensitivity: IC50 ≥ 1 uM
(M) moderately sensitive: 1 uM > IC50 ≥ 0.1 uM
(V) very sensitive: IC50 < 0.1 uM

### In Vivo NSCLC K-Ras G12C Xenograft Models

### Compound A:

### Methods:

The effects of a compound of the present invention, Compound A (H358 pERK K-Ras G12C EC50: 0.001 uM), on tumor cell growth in vivo were evaluated in the human non-small cell lung cancer NCI-H358 KRASG12C xenograft model using female BALB/c nude mice (6-8 weeks old). Mice were implanted with NCI-H358 tumor cells in 50% Matrigel (5×106 cells/mouse) subcutaneously in the flank. At the indicated tumor volume (dotted line, FIG. 2A), mice were randomized to treatment groups to start the administration of test articles or vehicle. Compound A was administered by oral gavage daily at the dose of 100 mg/kg. Body weight and tumor volume (using calipers) was measured twice weekly until study endpoints. Spaghetti plot (FIG. 2B) shows the tumor volume change in individual tumors during the course of treatment.

### Results:

FIG. 2A shows Compound A dosed at 100 mg/kg by daily oral gavage led to tumor regression in NCI-H358 KRASG12C xenograft model, which is a sensitive model to KRASG12C inhibition alone. The spaghetti titer plot (FIG. 2B) displaying individual tumor growth is shown next to the tumor volume plot (FIG. 2A). Over the treatment course of 28 days, Compound A drove tumor regression in all 10 animals bearing NCI-H358 KRASG12C tumors.

### Compound B:

### Methods:

The combinatorial effect of a compound of the present invention, Compound B (H358 pERK K-Ras G12C EC50: 0.003 uM), with cobimetinib on tumor cell growth in vivo were evaluated in the human non-small cell lung cancer NCI-H358 KRASG12C xenograft model using female BALB/c nude mice (6-8 weeks old). Mice were implanted with NCI-H358 tumor cells in 50% Matrigel (5×106 cells/mouse) subcutaneously in the flank. At indicated tumor volume (dotted line, FIG. 3A), mice were randomized to treatment groups to start the administration of test articles or vehicle. Compound B was administered by intermittent (twice weekly) intravenous injection at the dose of 50 mg/kg. Cobimetinib was administered by daily oral gavage at 2.5 mg/kg. The combination of Compound B and cobimetinib at their respective single-agent dose and regimen was also tested. Body weight and tumor volume (using calipers) was measured twice weekly until study endpoints. End of study responses in individual tumors were plotted as a waterfall plot (FIG. 3B), and the numbers indicate number of tumor regression in each group. Tumor regression is defined as greater than 10% reduction of tumor volume at the end of study relative to initial volume.

### Results:

FIG. 3A shows the combination of intermittent intravenous administration of Compound B at 50 mg/kg plus daily oral administration of cobimetinib at 2.5 mg/kg drove tumor regression, whereas each single agent led to tumor growth inhibition. End of study responses were shown as waterfall plots (FIG. 3B), which indicate 6 out 10 mice had tumor regression in the combination group, whereas no tumor regressions recorded in each single agent group.

### Compound C:

### Methods:

The combinatorial effect of a compound of the present invention, Compound C (H358 pERK K-Ras G12C EC50: 0.007 uM), with a SHP2 inhibitor, RMC-4550, on tumor cell growth in vivo were evaluated in the human non-small cell lung cancer NCI-H358 KRASG12C xenograft model using female BALB/c nude mice (6-8 weeks old). Mice were implanted with NCI-H358 tumor cells in 50% Matrigel (5×106 cells/mouse) subcutaneously in the flank. At indicated tumor volume (dotted line, FIG. 4A), mice were randomized to treatment groups to start the administration of test articles or vehicle. Compound C was administered by once weekly intravenous injection at the dose of 60 mg/kg. SHP2 inhibitor was administered by daily oral gavage at 30 mg/kg. The combination of Compound C and SHP2 inhibitor at their respective single-agent dose and regimen was also tested. Body weight and tumor volume (using calipers) was measured twice weekly until study endpoints. End of study responses in individual tumors were plotted as a waterfall plot (FIG. 4B), and the numbers indicate number of tumor regression in each group. Tumor regression is defined as greater than 10% reduction of tumor volume at the end of study relative to initial volume.

### Results:

In FIG. 4A, the combinatorial activity of once weekly intravenous administration of Compound C at 60 mg/kg plus daily oral administration of SHP2 inhibitor at 30 mg/kg is shown. The combination treatment had similar anti-tumor activity as the single agent SHP2 inhibitor, but the combination treatment led to 8 out of 10 mice with tumor regression, whereas single agent SHP2 inhibitor led to 5 out of 10 mice with tumor regressions. Single agent Compound C administered once weekly via intravenous injection led to tumor growth inhibition with one tumor regression.

### Cell Proliferation Assay

### Methods:

NCI-H358 cells were plated in 12-well tissue culture plates at a density of 100,000 cells/well in RPMI 1640 (10% FBS, 1% PenStrep) and cultured overnight at 37° C., 5% CO2. The following day, cells were treated with either trametinib (10 nM) or a compound of the present invention, Compound D (H358 pERK K-Ras G12C EC50: 0.024 uM), (17 nM). These concentrations represent the EC50 values from a 72-hour proliferation assay using the CellTiter-Glo® reagent (Promega). Additionally, cells were treated with the combination of trametinib and Compound D at the above indicated concentrations. The plate was placed in the Incucyte S3 live cell analysis system (37° C., 5% CO2) and confluence was measured by recording images at 6-hour intervals for a maximum of 40 days, or until wells reached maximal confluence. Media and drug were replaced at 3-4 day intervals. Data are plotted as % confluence over the time course of the experiment for each single agent and respective combination (FIG. 5).

### Results:

As shown in FIG. 5, treatment of NCI-H358 cells with submaximal (EC50) concentrations of Compound D or MEK inhibitor results in a short period of growth inhibition, followed by proliferation. Cells reach maximal confluence in −10 days after the addition of drug. The combination of the MEK inhibitor, trametinib, with Compound D resulted in complete and sustained inhibition of cell growth throughout the duration of the assay.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### APPENDIX C-1

### Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### SUMMARY

Provided herein are Ras inhibitors. The approach described herein entails formation of a high affinity three-component complex between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex between the Ras protein and the widely expressed cytosolic chaperone, cyclophilin A (CYPA). Without being bound by theory, the inventors believe that one way the inhibitory effect on Ras is effected by compounds of the invention and the complexes they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

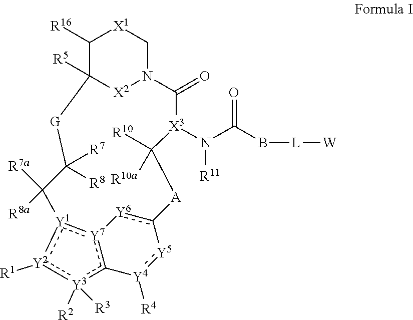

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₃ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;

R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl. Also provided are pharmaceutical compositions comprising a compound of Formula I, or a pharmaceutically acceptable salt thereof, and a pharmaceutically acceptable excipient; and

R¹⁶ is hydrogen or C₁-C₃ alkyl (e.g., methyl).

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE FIGURES

FIG. 1A: A compound of the present invention, Compound A, exhibits PK-dependent RAS pathway modulation in a Capan-2 CDX model (PDAC, KRAS G12V/WT). Single dose compared to twice administered PK/PD measurement of Compound A. Second dose of Compound A delivered 8 hours following first dose, depicted by black arrow. All dose levels well tolerated. Tumor DUSP6 mRNA expression as percent of control graphed as bars on left y-axis. Dotted line indicates return to control level of DUSP6. Unbound plasma PK (nM) graphed as lines, plotted in Log 10 scale on right y-axis. N=3/time point. Error bars represent standard error of the mean.

FIG. 1B: Combinatorial anti-tumor activity with a compound of the present invention, Compound A, and upstream SHP2 inhibition in a Capan-2 CDX model (PDAC, KRAS G12V/WT). Capan-2 cells were implanted in 50% Matrigel. Animals were randomized and treatment was initiated at average tumor volume of ˜180 mm3. Animals were dosed with SHP2 inhibitor RMC-4550 20 mg/kg po q2d, Compound A 100 mg/kg po bid, combination RMC-4550 and Compound A, or Control for 40 days. All dose levels were tolerated. n=10/group (n=9 in Combination arm). Ns=no significance; ***p<0.001 by one-way ANOVA.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₆ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O) NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)_{0- 4}S(O)₂R^{∘}; —(CH₂)₀₋₄S(O)₂₀R^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —N O₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}2, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

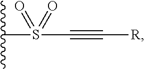

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

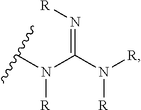

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting moiety B to moiety W in a compound of Formula I, such that the resulting compound is capable of achieving an IC50 of 2 uM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD}D construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

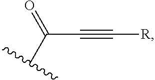

wherein R is any any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex between the Ras protein and the widely expressed cytosolic chaperone, cyclophilin A (CYPA). Without being bound by theory, the inventors believe that one way the inhibitory effect on Ras is effected by compounds of the invention and the complexes they form is by steric occlusion of the interaction site between Ras and downstream effector molecules, such as RAF, which are required for propagating the oncogenic signal.

Without being bound by theory, the inventors postulate that non-covalent interactions of a compound of the present invention with Ras and the chaperone protein (e.g., cyclophilin A) may contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Accordingly, provided herein is a compound, or pharmaceutically acceptable salt thereof, having the structure of Formula 00:

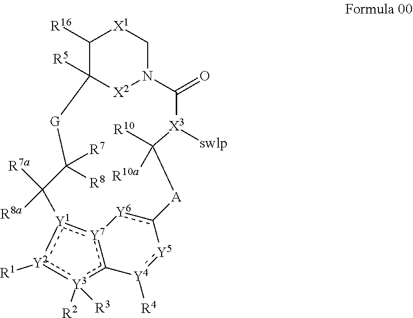

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

swIp (Switch I/P-loop) refers to an organic moiety that non-covalently binds to both the Switch I binding pocket and residues 12 or 13 of the P-loop of a Ras protein (see, e.g., Johnson et al., 292:12981-12993 (2017), incorporated herein by reference);

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;

R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R¹⁰ is hydrogen, halo, hydroxy, C₁-C₃ alkoxy, or C₁-C₃ alkyl;

R^{10a} is hydrogen or halo; and

R¹⁸ is hydrogen or C₁-C₃ alkyl (e.g., methyl). In some embodiments, the resulting compound is capable of achieving an IC50 of 2 uM or less (e.g., 1.5 uM, 1 uM, 500 nM, or 100 nM or less) in the Ras-RAF disruption assay protocol described herein.

Accordingly, provided herein is a compound, or pharmaceutically acceptable salt thereof, having the structure of Formula I:

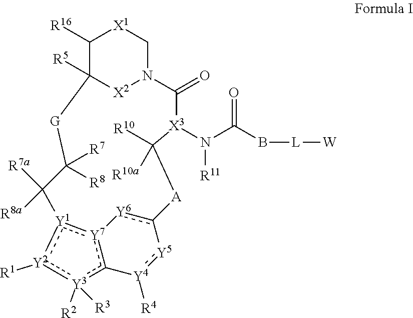

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is absent, —CH(R⁹)—, or >C═CR⁹R^{9′}, where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;

R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl;

R¹⁶ is hydrogen or C₁-C₃ alkyl (e.g., methyl).

In some embodiments, the disclosure features a compound, or pharmaceutically acceptable salt thereof, of structural Formula Ia:

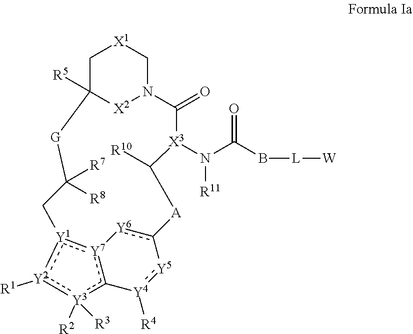

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;

R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo; and

R¹¹ is hydrogen or C₁-C₃ alkyl.

In some embodiments, the disclosure features a compound, or pharmaceutically acceptable salt thereof, of structural Formula Ib:

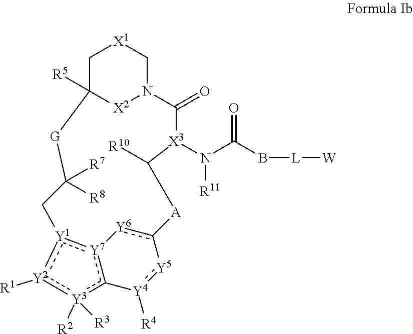

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments of compounds of the present invention, G is optionally substituted C₁-C₄ heteroalkylene.

In some embodiments, a compound of the present invention has the structure of Formula Ic, or a pharmaceutically acceptable salt thereof:

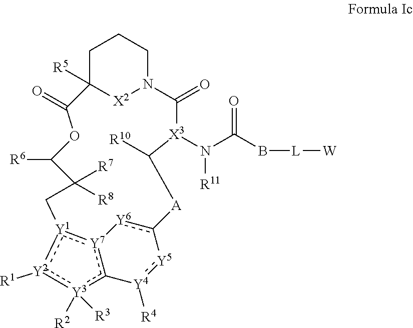

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;

R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

In some embodiments of compounds of the present invention, X² is NH. In some embodiments, X³ is CH.

In some embodiments of compounds of the present invention, R¹¹ is hydrogen. In some embodiments, R¹¹ is C₁-C₃ alkyl. In some embodiments, R¹¹ is methyl.

In some embodiments, a compound of the present invention has the structure of Formula Id, or a pharmaceutically acceptable salt thereof:

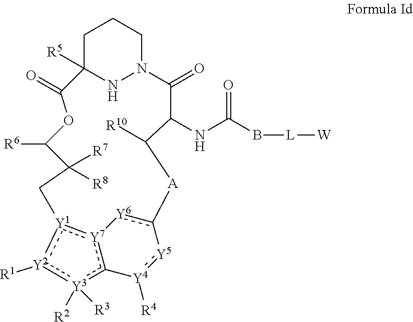

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—.

In some embodiments of compounds of the present invention, R³ is absent.

In some embodiments of compounds of the present invention, R⁴ is hydrogen.

In some embodiments of compounds of the present invention, R⁵ is hydrogen. In some embodiments, R⁵ is C₁-C₄ alkyl optionally substituted with halogen. In some embodiments, R⁵ is methyl.

In some embodiments of compounds of the present invention, Y⁴ is C. In some embodiments, Y⁵ is CH. In some embodiments, Y⁶ is CH. In some embodiments, Y¹ is C. In some embodiments, Y² is C.

In some embodiments, Y³ is N. In some embodiments, Y⁷ is C.

In some embodiments, a compound of the present invention has the structure of Formula Ie, or a pharmaceutically acceptable salt thereof:

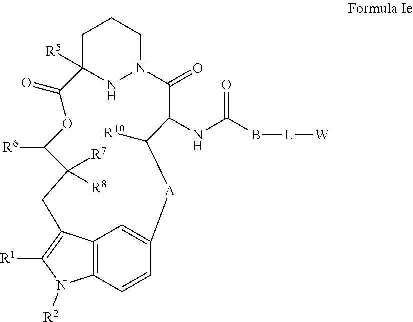

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or

R² and R³ combine with the atom to which they are attached to form an optionally substituted 3 to 8-membered cycloalkyl or optionally substituted 3 to 14-membered heterocycloalkyl;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of compounds of the present invention, R⁶ is hydrogen.

In some embodiments of compounds of the present invention, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl. In some embodiments, R² is fluoro C₁-C₆ alkyl, such as —CH₂CH₂F, —CH₂CHF₂, or —CH₂CF₃.

In some embodiments of compounds of the present invention, R⁷ is optionally substituted C₁-C₃ alkyl. In some embodiments, R⁷ is C₁-C₃ alkyl.

In some embodiments of compounds of the present invention, R⁸ is optionally substituted C₁-C₃ alkyl. In some embodiments, R⁸ is C₁-C₃ alkyl, such as methyl.

In some embodiments, a compound of the present invention has the structure of Formula If, or a pharmaceutically acceptable salt thereof:

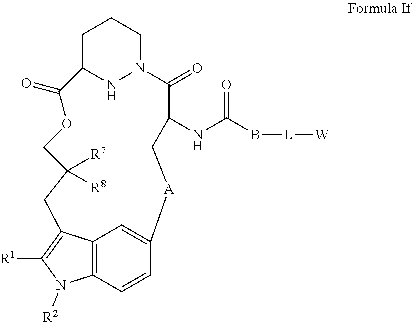

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of compounds of the present invention, R¹ is 5 to 10-membered heteroaryl.

In some embodiments, R¹ is optionally substituted 6-membered aryl or optionally substituted 6-membered heteroaryl.

In some embodiments of compounds of the present invention, R₁ is

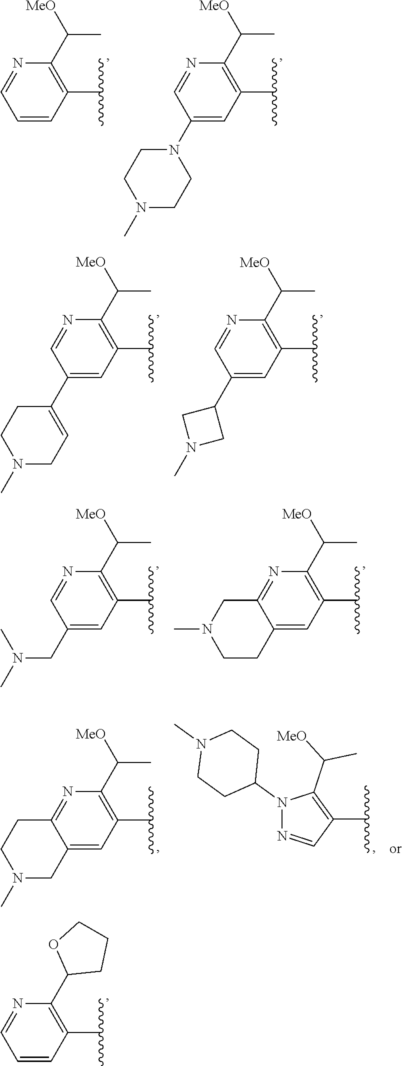

or a stereoisomer thereof. In some embodiments, R₁ is

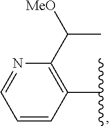 [SMILES: *c1cccnc1C(C)OC]

or a stereoisomer thereof. In some embodiments, R₁ is

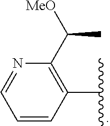 [SMILES: *c1cccnc1[C@H](C)OC]

In some embodiments, R₁ is

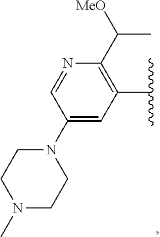 [SMILES: *c1cc(N2CCN(C)CC2)cnc1C(C)OC]

or a stereoisomer thereof. In some embodiments, R₁ is

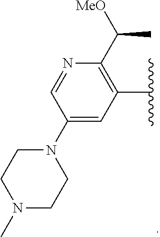 [SMILES: *c1cc(N2CCN(C)CC2)cnc1[C@H](C)OC]

In some embodiments, a compound of the present invention has the structure of Formula Ig, or a pharmaceutically acceptable salt thereof:

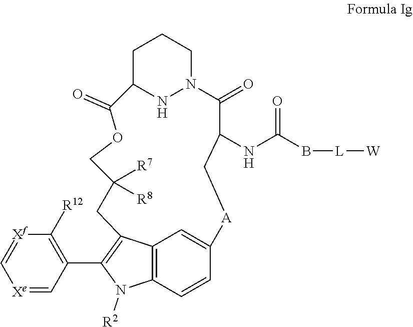

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R² is C₁-C₆ alkyl or 3 to 6-membered cycloalkyl;

R⁷ is C₁-C₃ alkyl;

R⁸ is C₁-C₃ alkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} is N, CH, or CR¹⁷;

X^{f} is N or CH;

R¹² is optionally substituted C₁-C₆ alkyl or optionally substituted C₁-C₆ heteroalkyl; and

R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments of compounds of the present invention, X^{e} is N and X^{f} is CH. In some embodiments, X^{e} is CH and X^{f} is N. In some embodiments, X^{e} is CR¹⁷ and X^{f} is N.

In some embodiments of compounds of the present invention, R¹² is optionally substituted C₁-C₆ heteroalkyl. In some embodiments, R¹² is

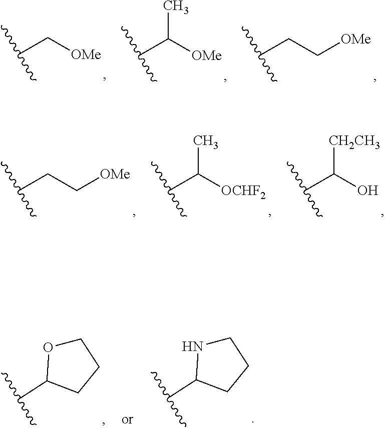

In some embodiments, a compound of the present invention has the structure of Formula Ih, or a pharmaceutically acceptable salt thereof:

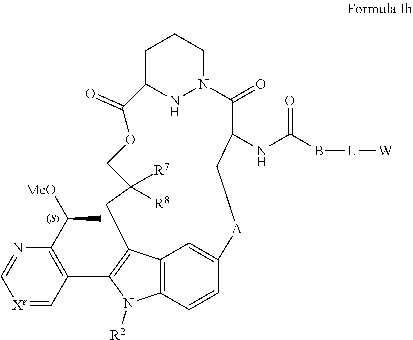

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R² is C₁-C₆ alkyl or 3 to 6-membered cycloalkyl;

R⁷ is C₁-C₃ alkyl;

R⁸ is C₁-C₃ alkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X¹ is CH, or CR¹⁷; and

R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, a compound of the present invention has the structure of Formula Ii, or a pharmaceutically acceptable salt thereof:

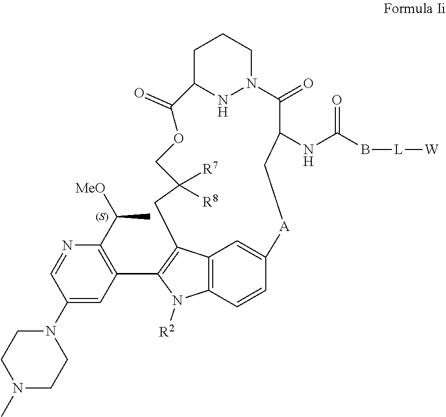

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of compounds of the present invention, A is optionally substituted 6-membered arylene. In some embodiments, A has the structure:

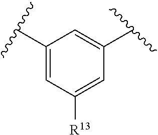

wherein R¹³ is hydrogen, hydroxy, amino, cyano, optionally substituted C₁-C₆ alkyl, or optionally substituted C₁-C₆ heteroalkyl. In some embodiments, R¹³ is hydrogen. In some embodiments, hydroxy. In some embodiments, A is an optionally substituted 5 to 10-membered heteroarylene. In some embodiments, A is:

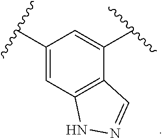 [SMILES: *c1cc(*)c2cn[nH]c2c1]

In some embodiments, A is optionally substituted 5 to 6-membered heteroarylene. In some embodiments, A is:

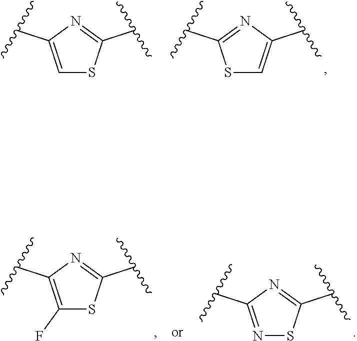 [SMILES: *c1csc(*)n1 ; *c1csc(*)n1 ; *c1nc(*)c(F)s1 ; *c1nsc(*)n1]

In some embodiments, A is

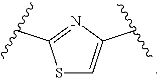 [SMILES: *c1csc(*)n1]

In some embodiments of compounds of the present invention, B is —CHR⁹—. In some embodiments, R⁹ is optionally substituted C₁-C₆ alkyl or optionally substituted 3 to 6-membered cycloalkyl. In some embodiments, R⁹ is:

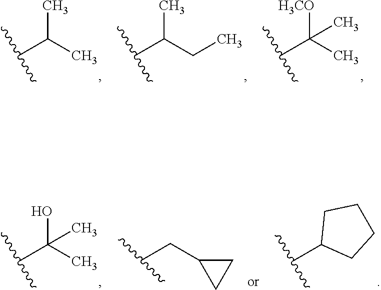

In some embodiments, R⁹ is:

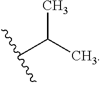 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, B is optionally substituted 6-membered arylene.

In some embodiments, B is 6-membered arylene. In some embodiments, B is:

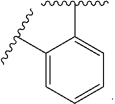 [SMILES: *c1ccccc1*]

In some embodiments B is absent.

In some embodiments of compounds of the present invention, R⁷ is methyl.

In some embodiments of compounds of the present invention, R⁸ is methyl.

In some embodiments of compounds of the present invention, R¹⁸ is hydrogen.

In some embodiments of compounds of the present invention, the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A². In some embodiments, the linker is acyclic. In some embodiments, the linker has the structure of Formula IIa:

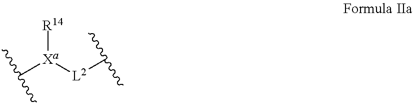

wherein X^{a} is absent or N;

R¹⁴ is absent, hydrogen or optionally substituted C₁-C₆ alkyl or optionally substituted C₁-C₃ cycloalkyl; and

L² is absent, —C(O)—, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

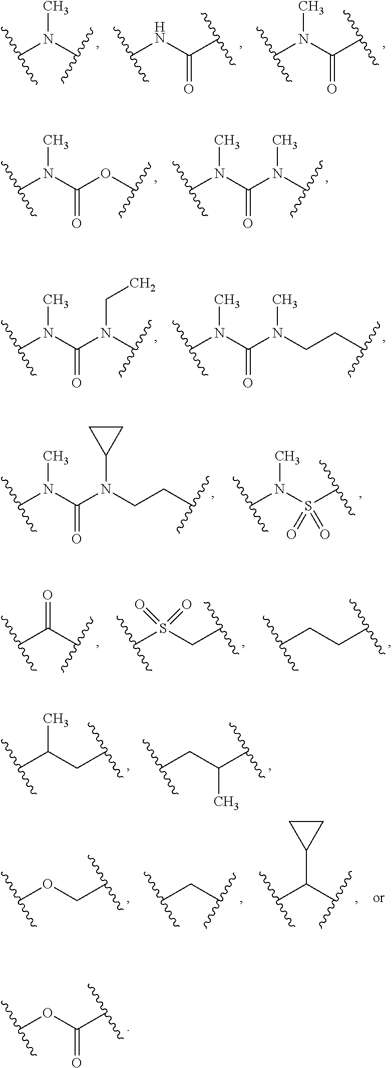

In some embodiments, L is

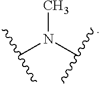 [SMILES: *N(*)C]

In some embodiments, L is

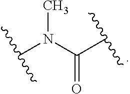 [SMILES: *C(=O)N(*)C]

In some embodiments, linker is or comprises a cyclic group. In some embodiments, linker has the structure of Formula IIb:

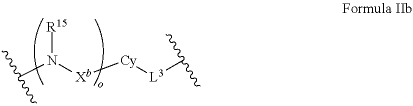

wherein o is 0 or 1;

X^{b} is C(H) or SO₂;

R¹⁵ is hydrogen or optionally substituted C₁-C₆ alkyl;

Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —C(O)—, —SO₂—, optionally substituted C₁-C₄ alkylene or optionally substituted C₁-C₄ heteroalkylene. In some embodiments, linker has the structure:

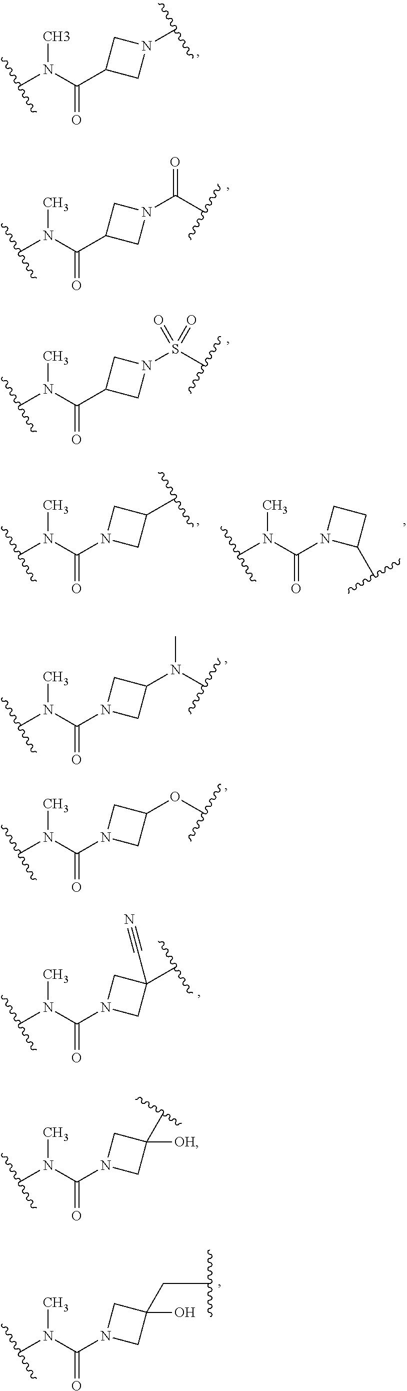

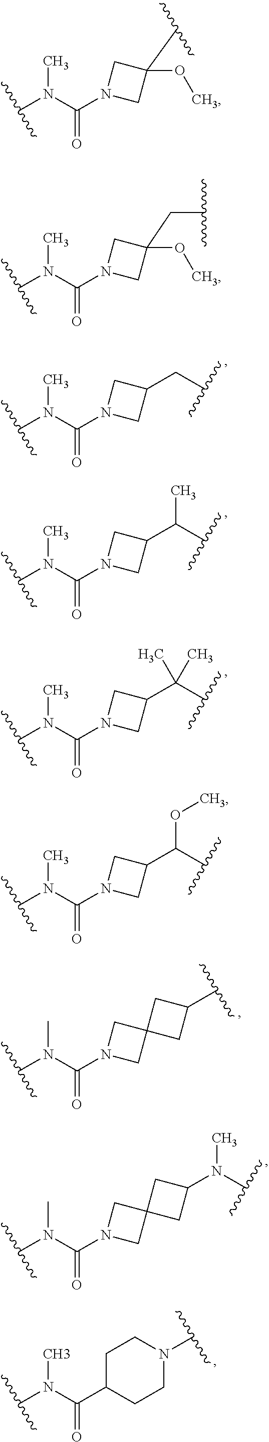

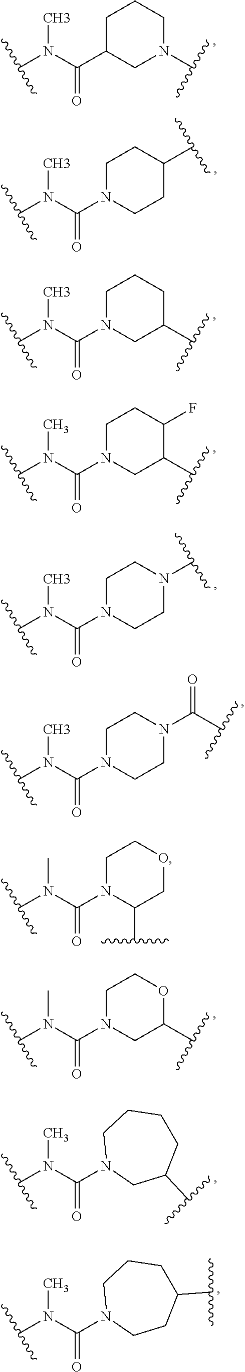

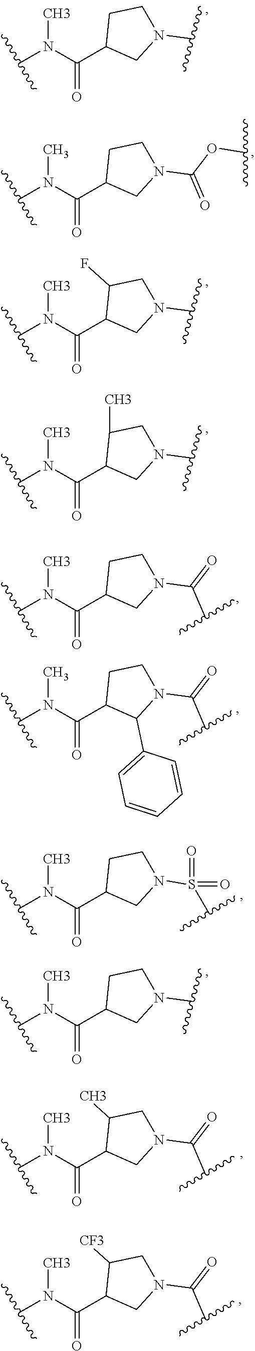

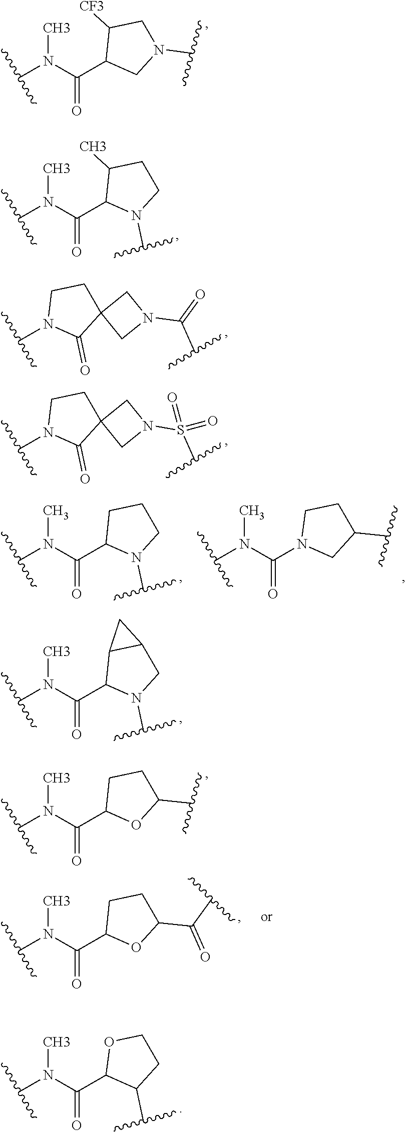

In some embodiments of compounds of the present invention, W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 8-membered heterocycloalkyl, optionally substituted 3 to 8-membered cycloalkyl, or 3 to 8-membered heteroaryl.

In some embodiments of compounds of the present invention, W is hydrogen. In some embodiments, W is optionally substituted amino. In some embodiments, W is —NHCH₃ or —N(CH₃)₂. In some embodiments, W is optionally substituted C₁-C₄ alkoxy. In some embodiments, W is methoxy or iso-propoxy. In some embodiments, W is optionally substituted C₁-C₄ alkyl. In some embodiments, W is methyl, ethyl, iso-propyl, tert-butyl, or benzyl. In some embodiments, W is optionally substituted amido. In some embodiments, W is

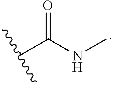 [SMILES: *C(=O)NC]

In some embodiments, W is optionally substituted amido. In some embodiments, W is

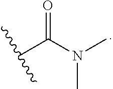 [SMILES: *C(=O)N(C)C]

In some embodiments, W is optionally substituted C₁-C₄ hydroxyalkyl. In some embodiments, W is

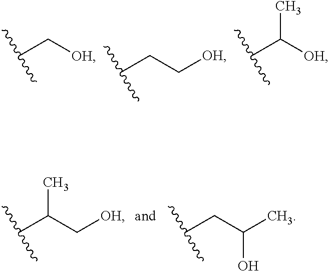

In some embodiments, W is optionally substituted C₁-C₄ aminoalkyl. In some embodiments, W is

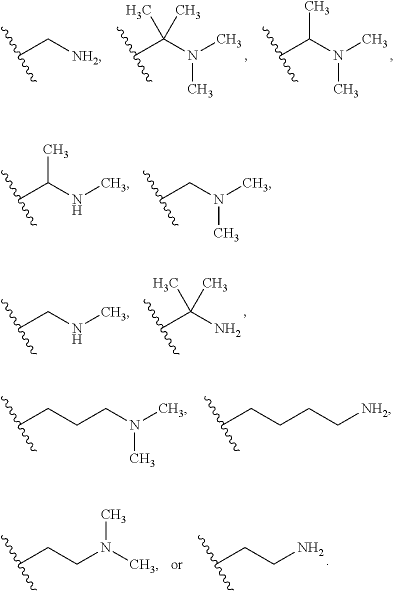

In some embodiments, W is optionally substituted C₁-C₄ haloalkyl. In some embodiments, W is

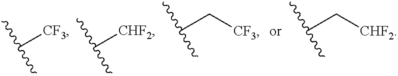

In some embodiments, W is optionally substituted C₁-C₄ guanidinoalkyl. In some embodiments, W is

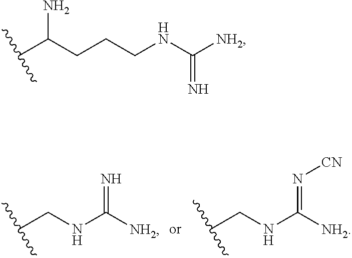 [SMILES: *C(N)CCCNC(=N)N ; *CNC(=N)N ; *CN/C(N)=N/C#N]

In some embodiments, W is C₀-C₄ alkyl optionally substituted 3 to 11-membered heterocycloalkyl. In some embodiments W is

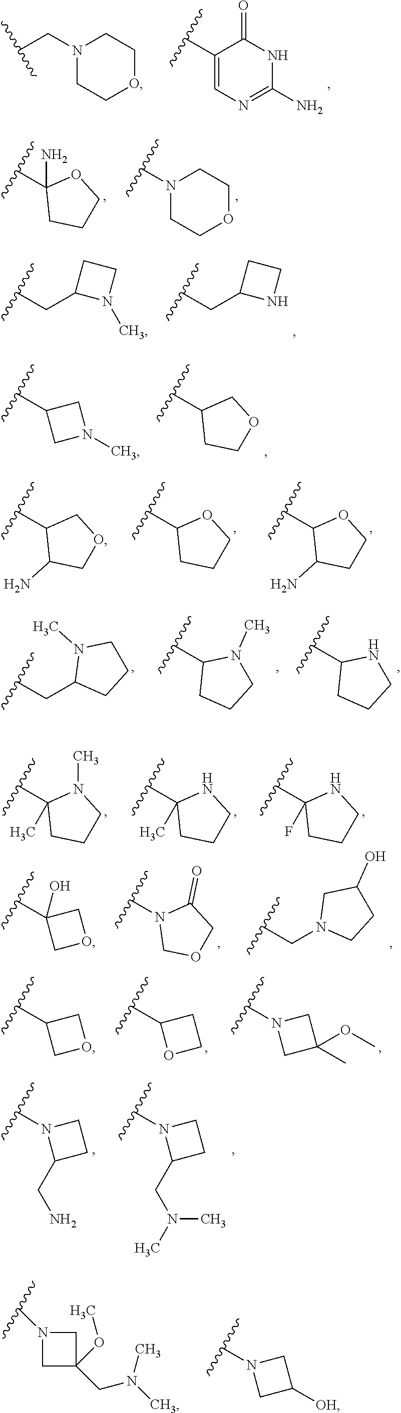

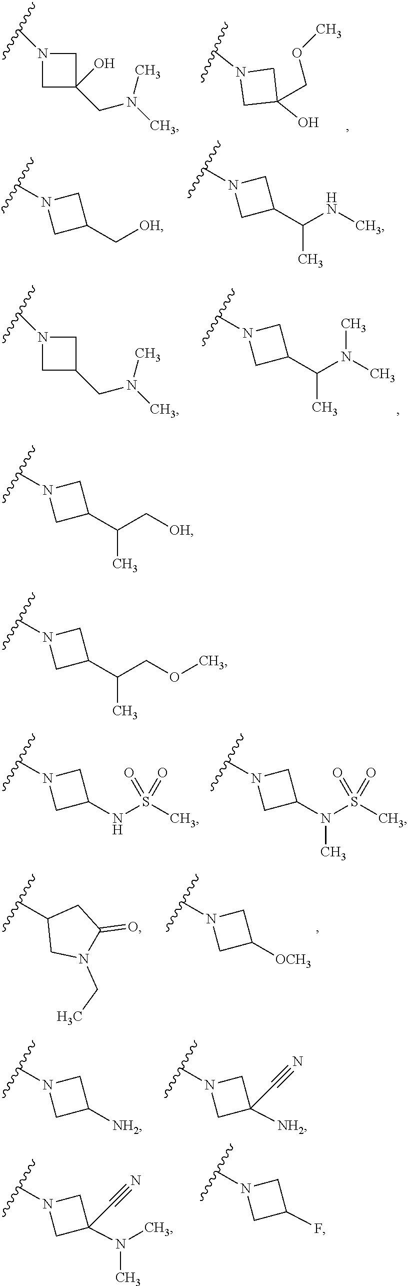

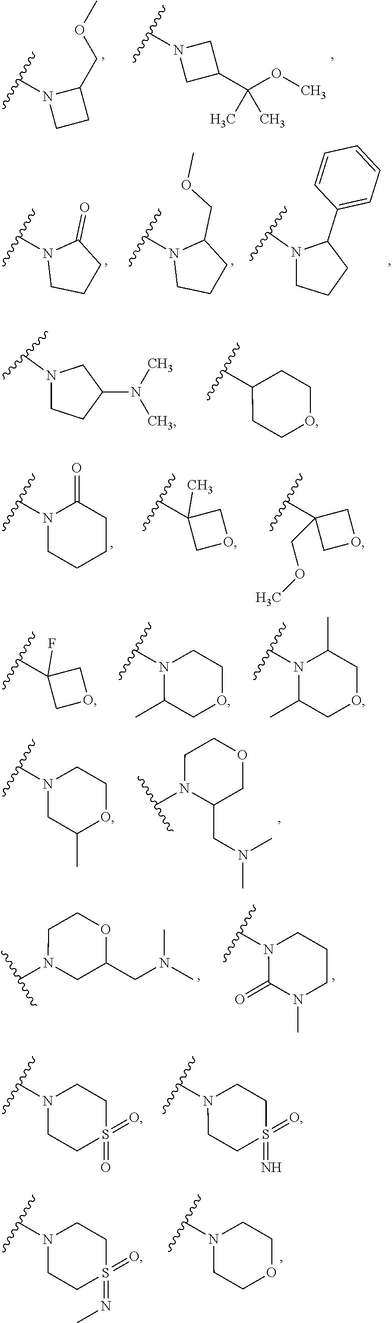

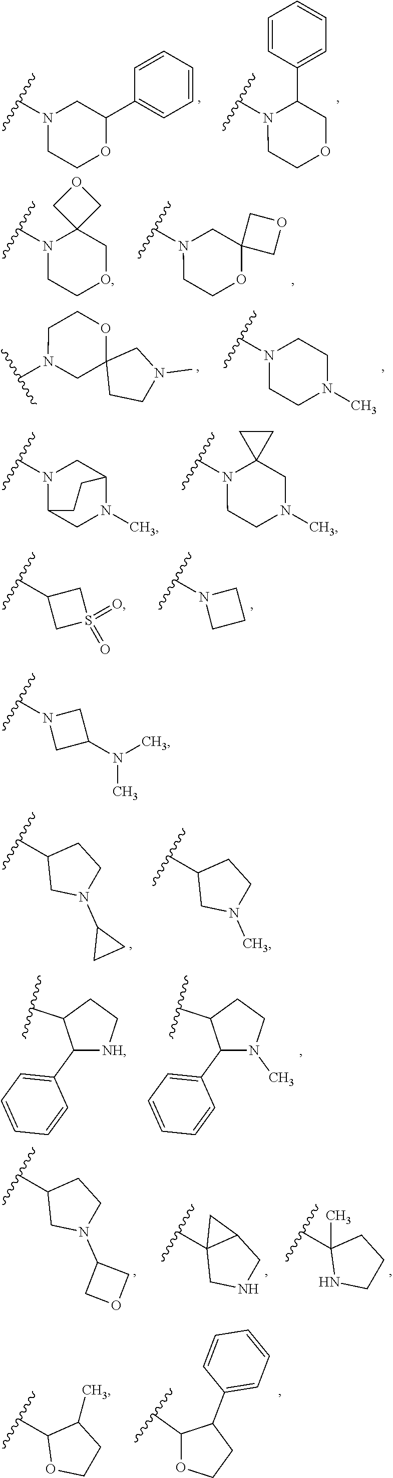

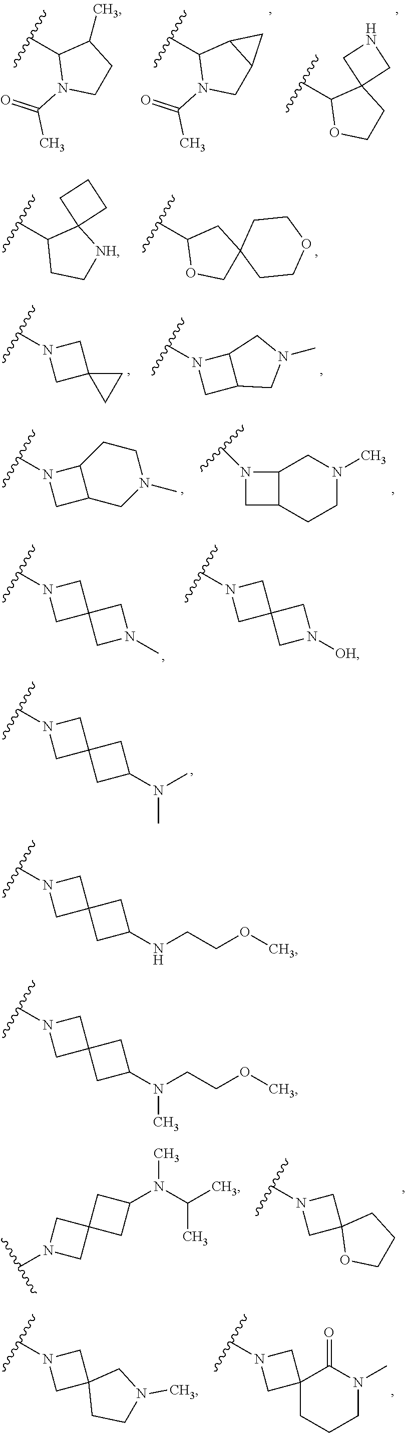

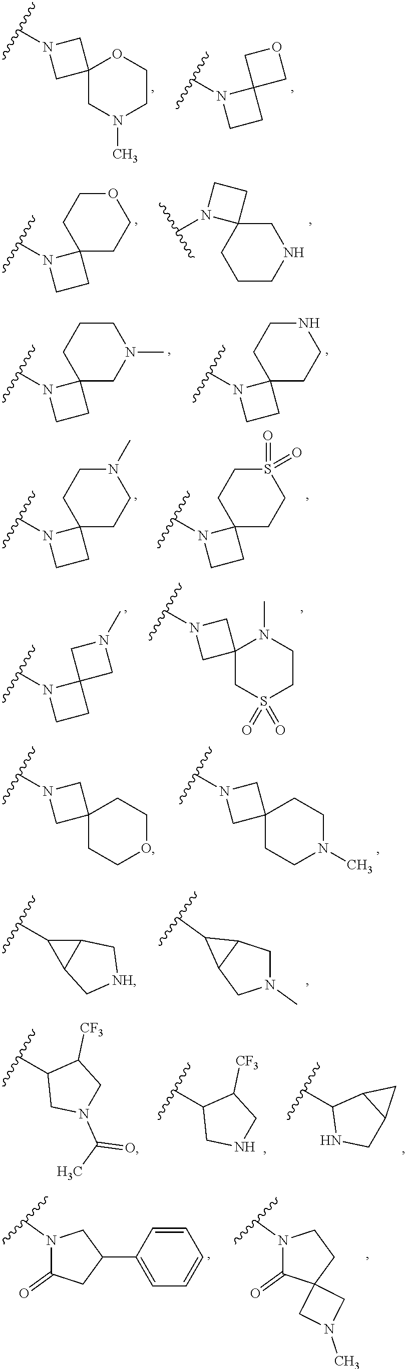

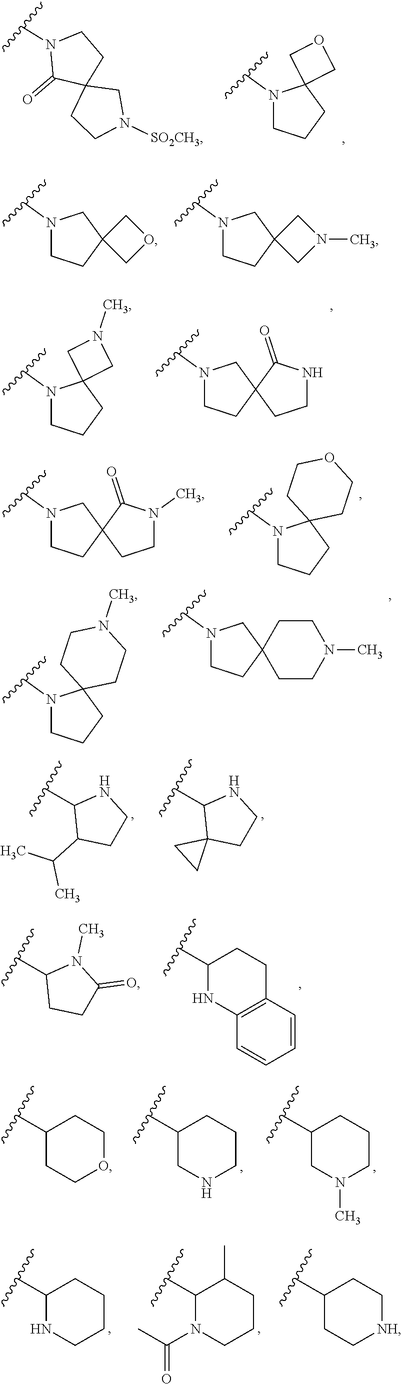

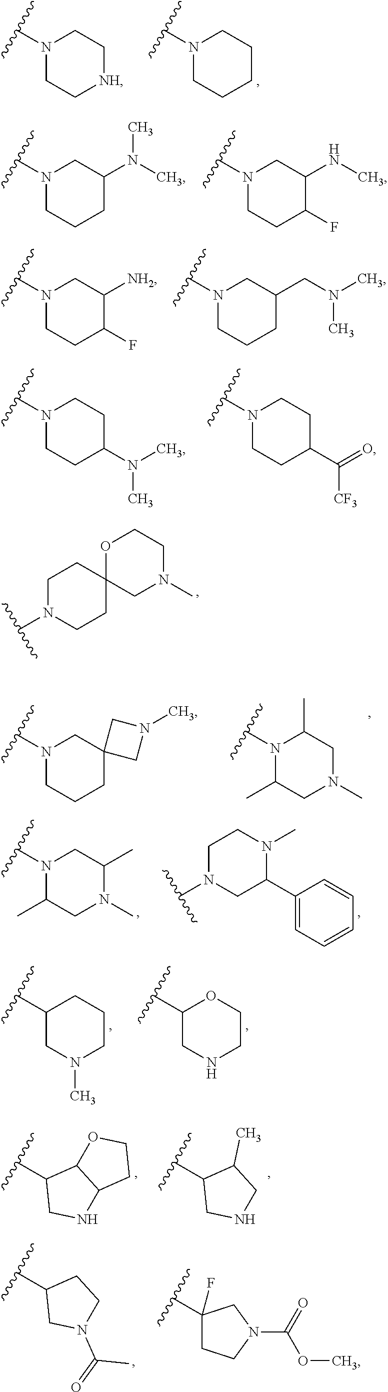

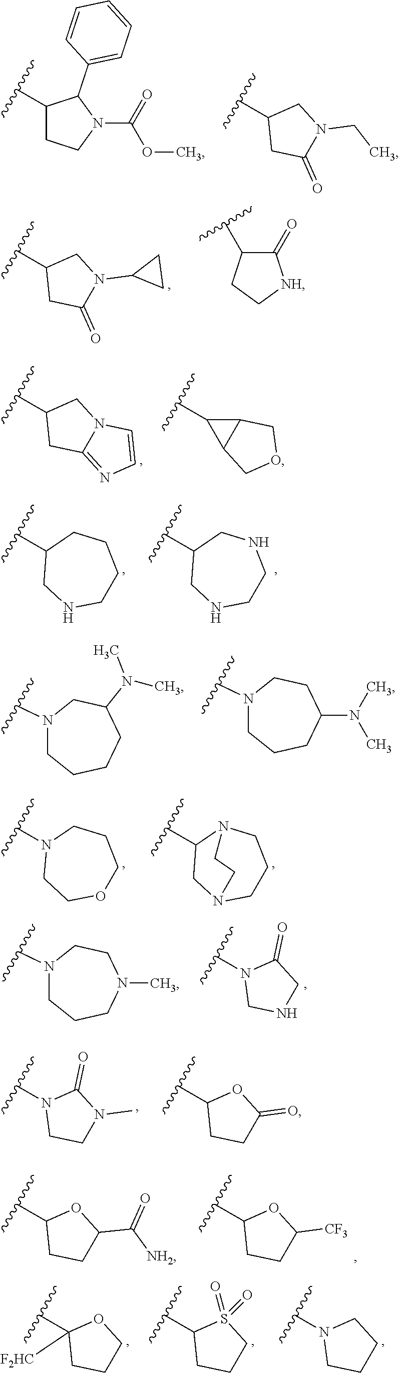

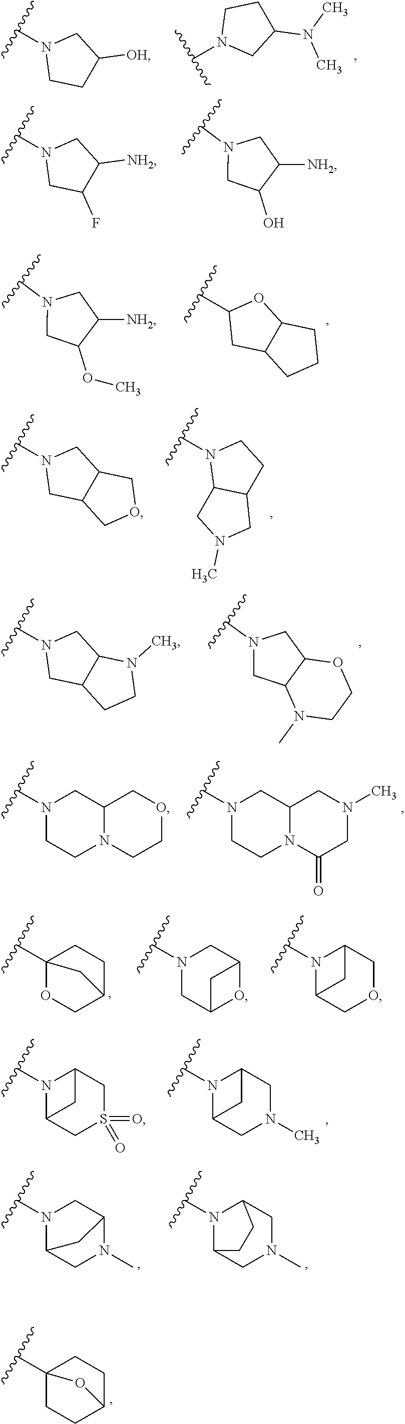

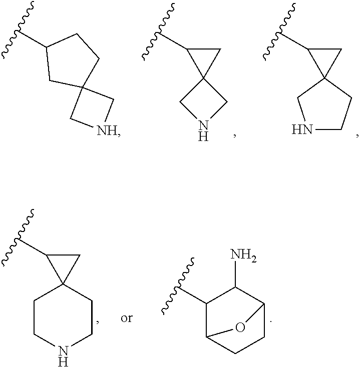 [SMILES: *C1CCC2(CNC2)C1 ; *C1CC12CNC2 ; *C1CC12CCNC2 ; *C1CC12CCNCC2 ; *C1C2CCC(O2)C1N]

In some embodiments, W is optionally substituted 3 to 8-membered cycloalkyl. In some embodiments, W is

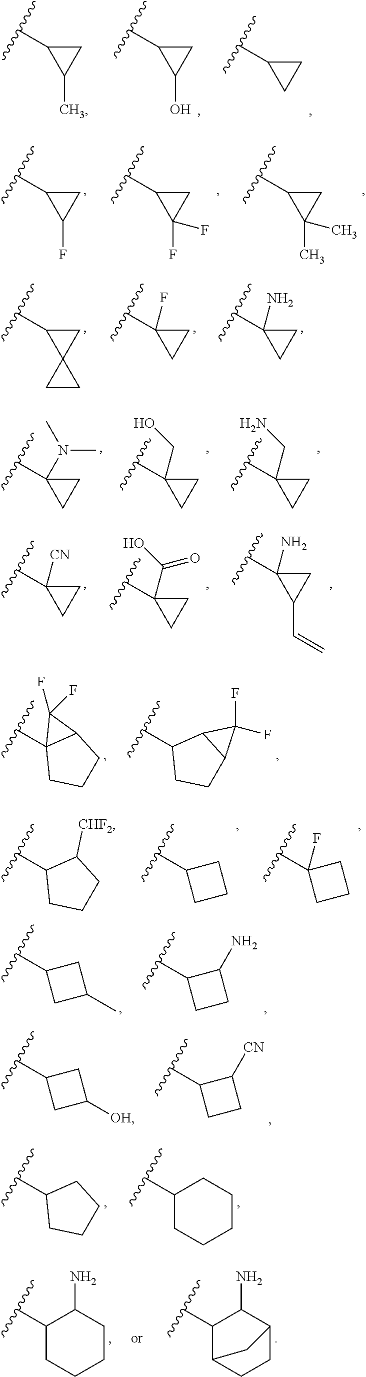

In some embodiments, W is optionally substituted 3 to 8-membered heteroaryl. In some embodiments, W is

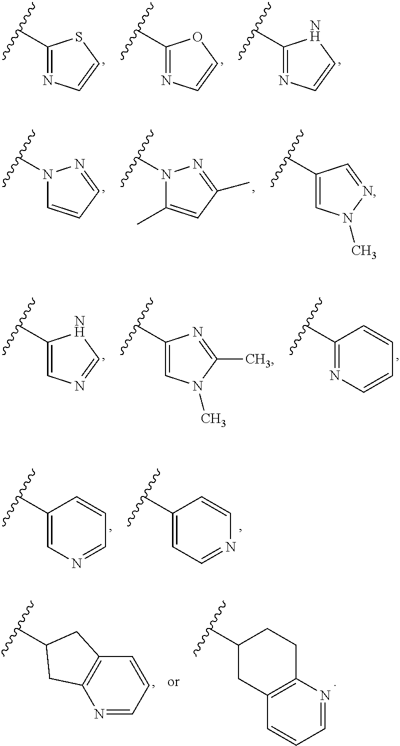

In some embodiments, W is optionally substituted 6- to 10-membered aryl (e.g., phenyl, 4-hydroxy-phenyl, or 2,4-methoxy-phenyl).

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present invention

| Ex # | Structure |
|---|---|
| A1 | 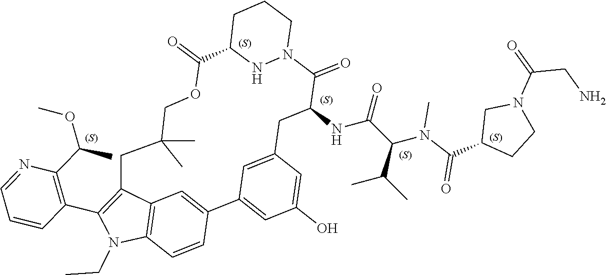 |
| A2 | 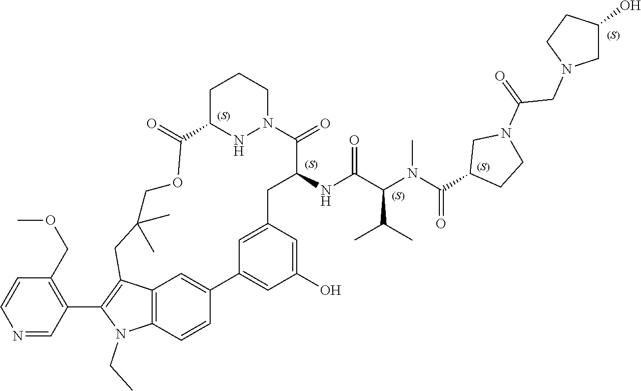 |
| A3 | 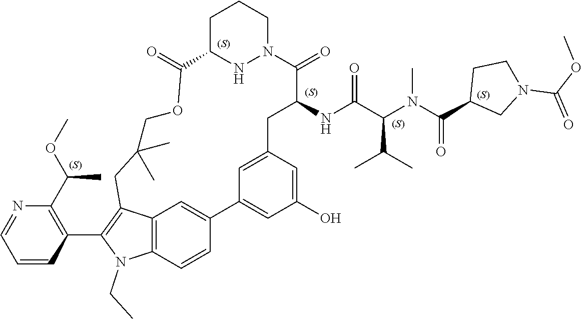 |
| A4 | 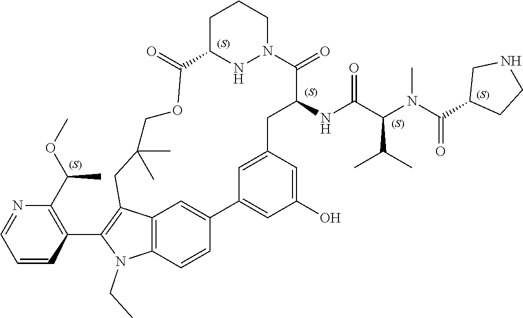 |
| A5 | 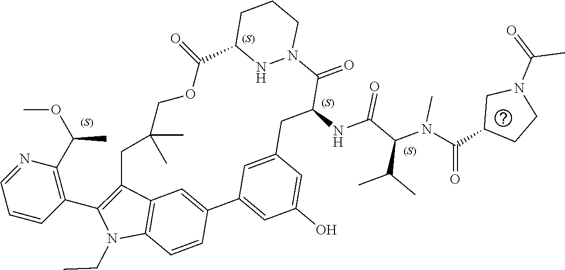 |
| A6 | 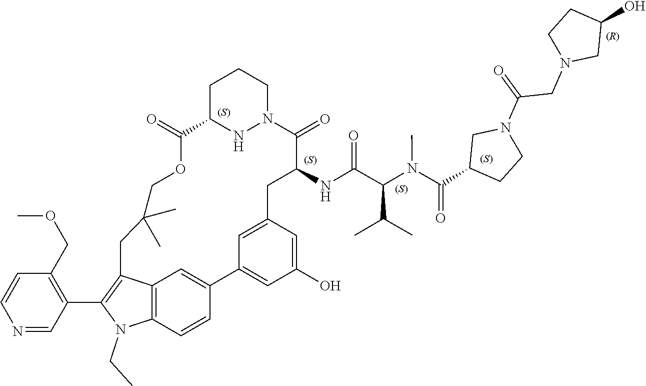 |
| A7 | 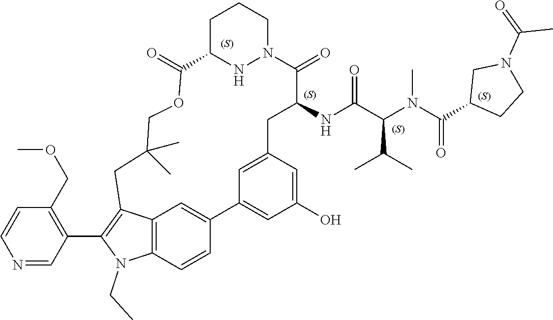 |
| A8 | 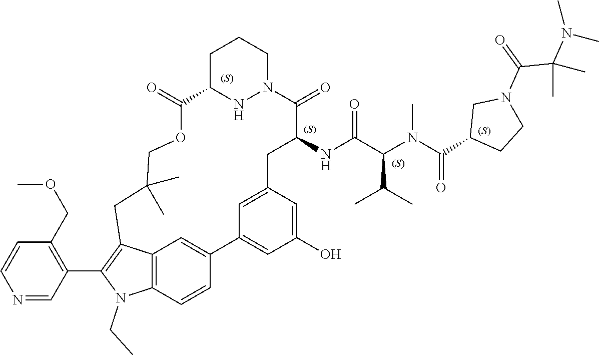 |
| A9 | 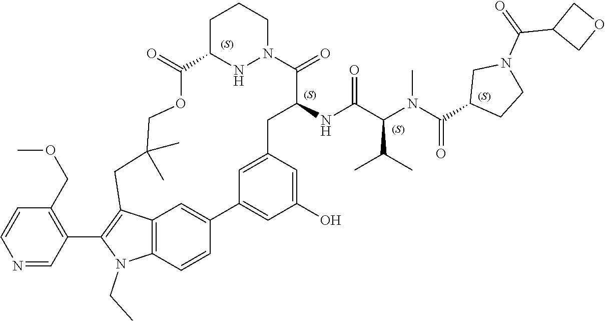 |
| A10 | 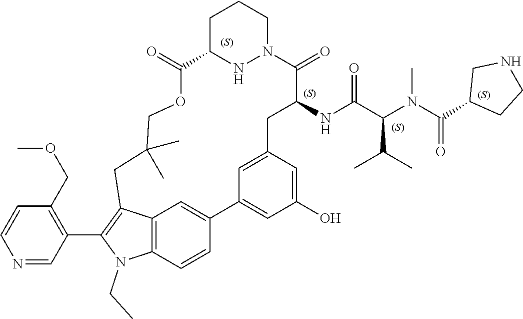 |
| A11 | 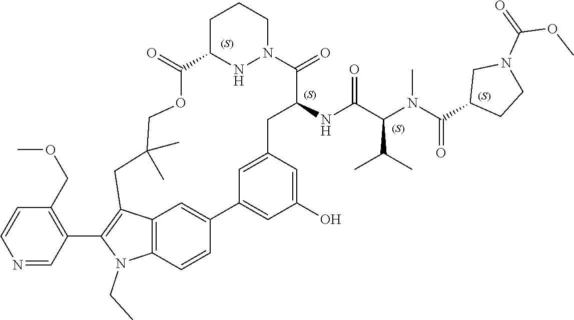 |
| A12 | 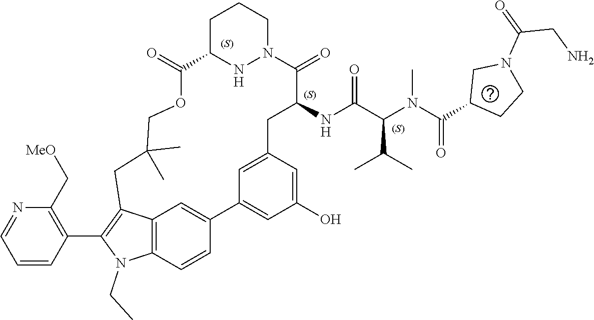 |
| A13 | 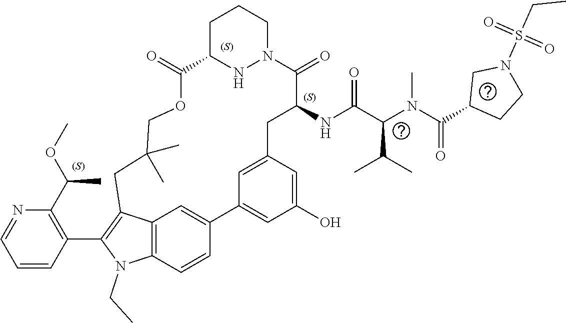 |
| A14 | 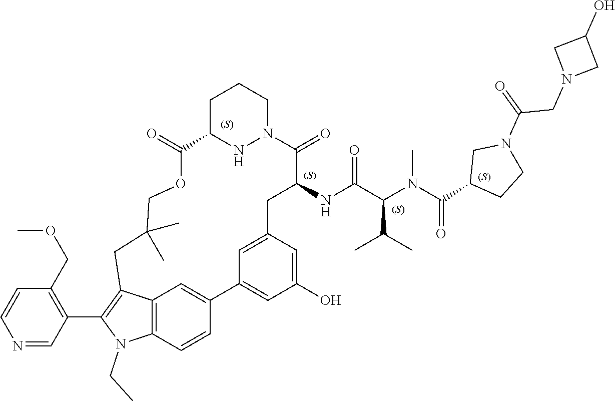 |
| A15 | 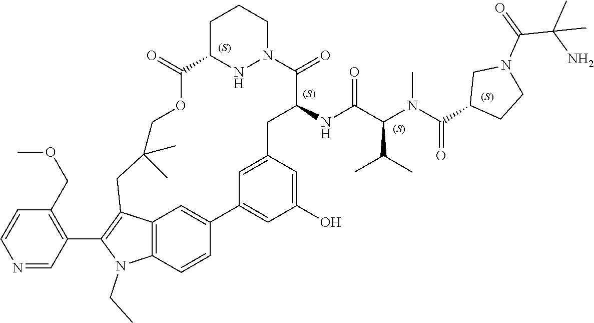 |
| A16 | 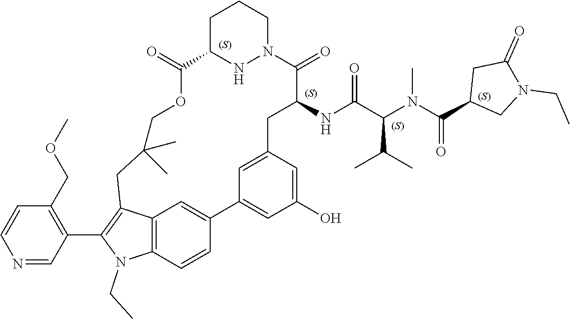 |
| A17 | 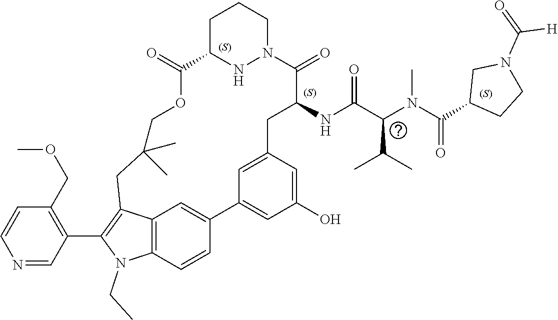 |
| A18 | 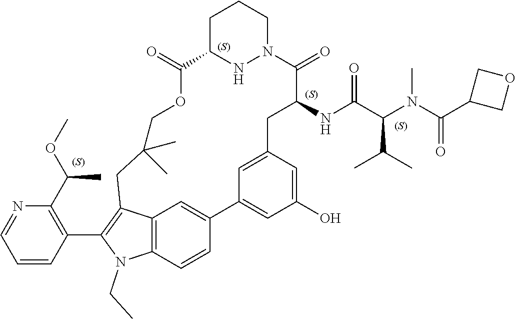 |
| A19 | 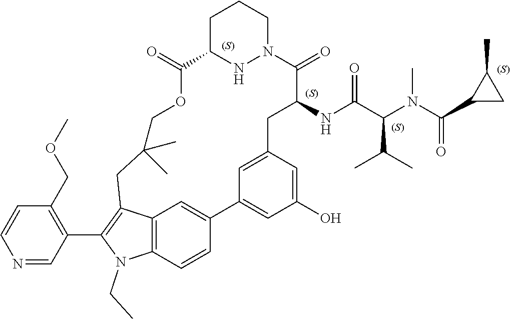 |
| A20 | 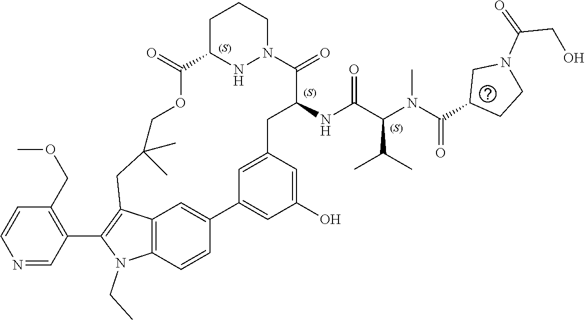 |
| A21 | 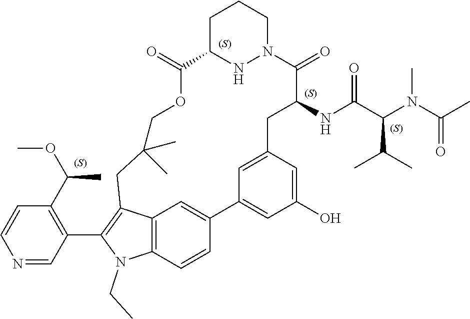 |
| A22 | 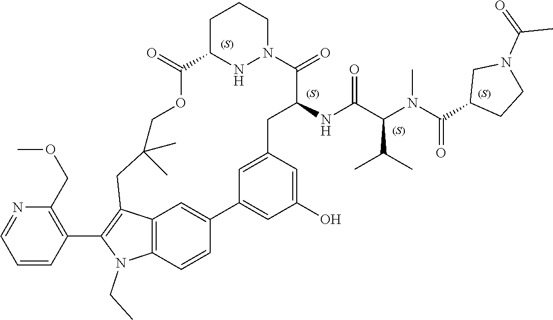 |
| A23 | 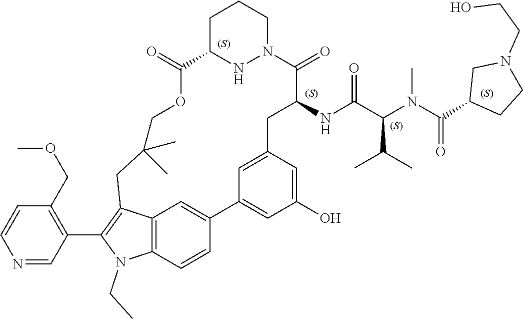 |
| A24 | 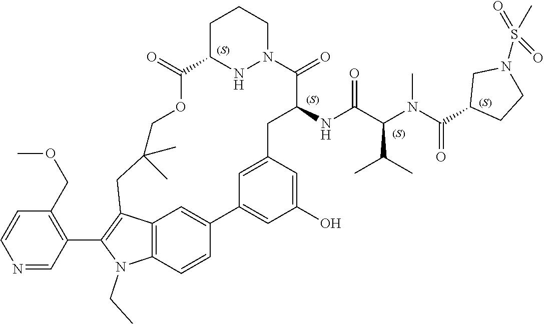 |
| A25 | 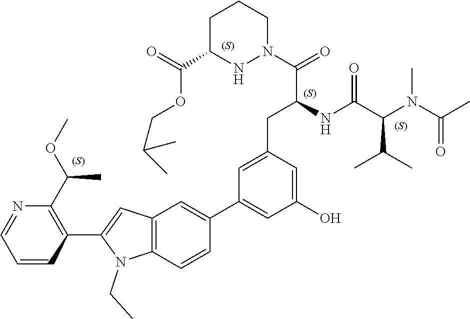 |
| A26 | 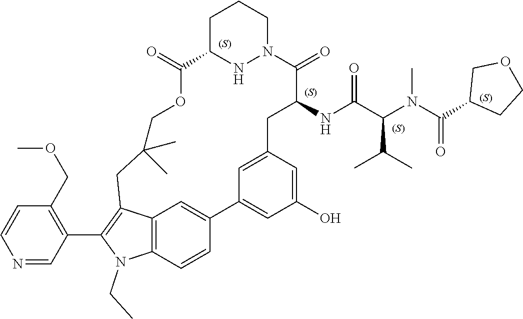 |
| A27 | 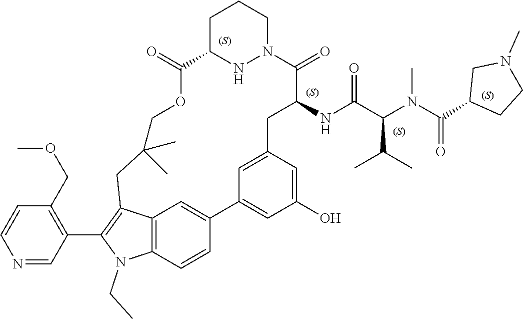 |
| A28 | 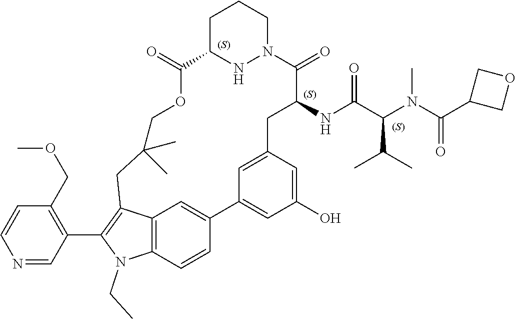 |
| A29 | 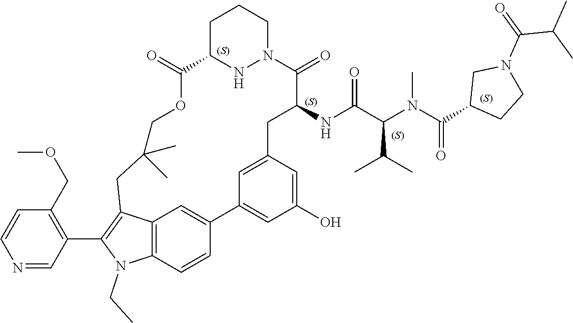 |
| A30 | 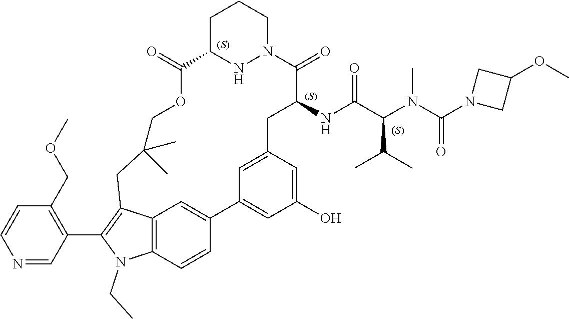 |
| A31 | 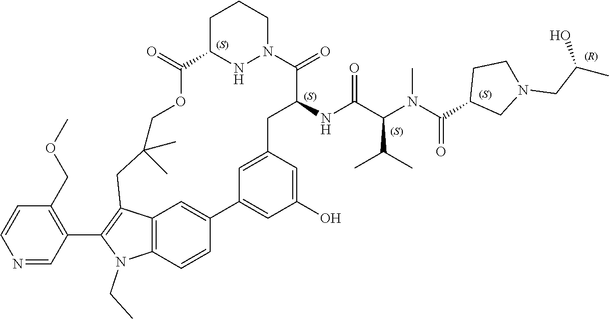 |
| A32 | 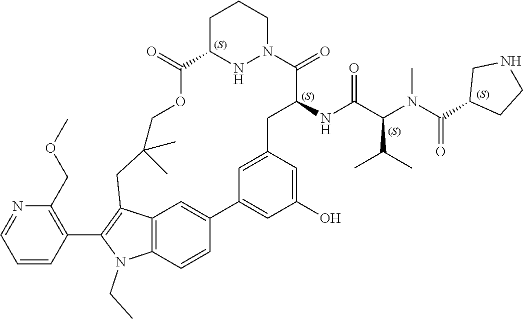 |
| A33 | 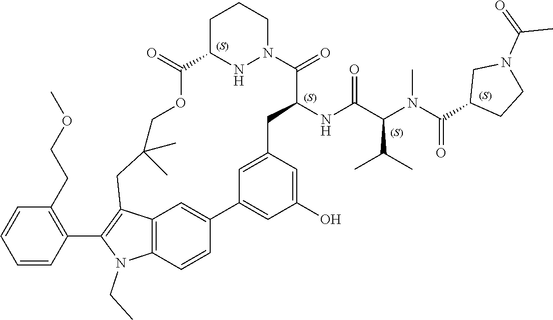 |
| A34 | 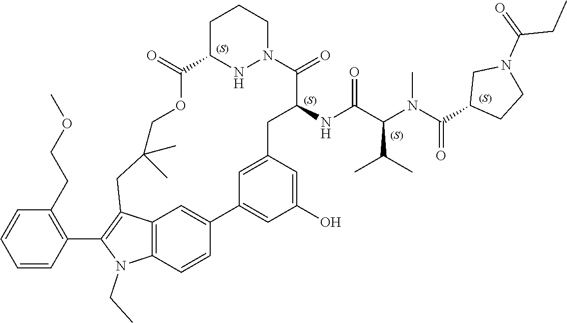 |
| A35 | 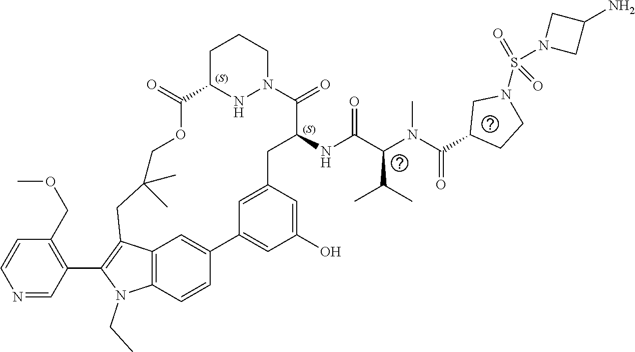 |
| A36 | 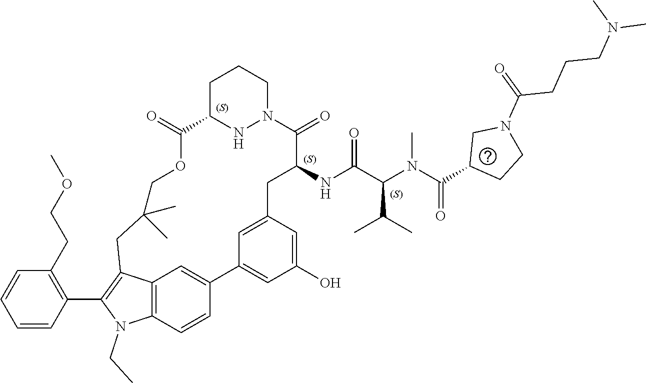 |
| A37 | 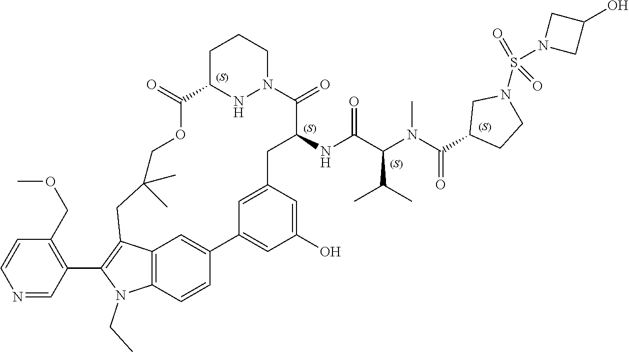 |
| A38 | 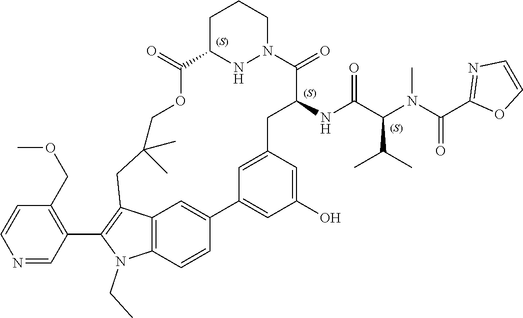 |
| A39 | 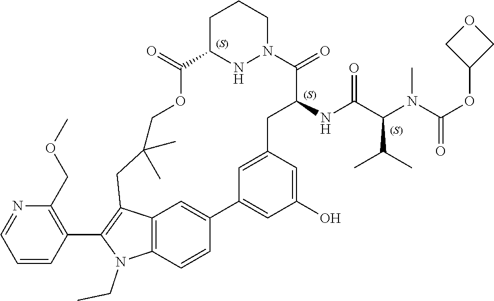 |
| A40 | 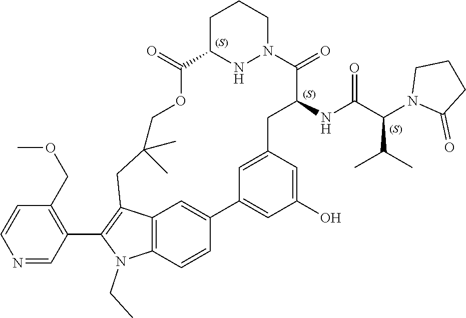 |
| A41 | 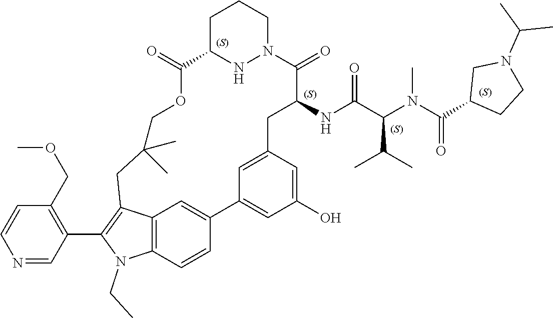 |
| A42 | 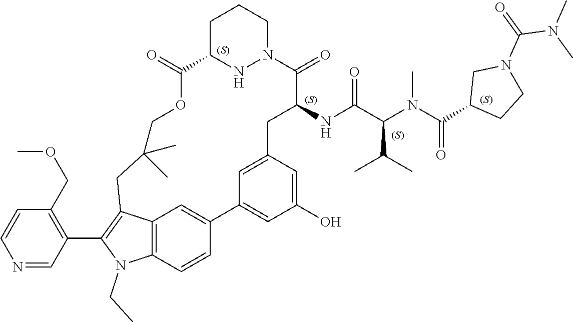 |
| A43 | 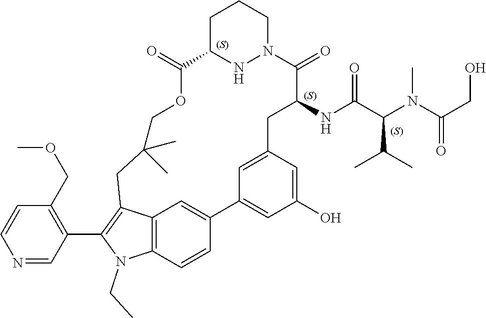 |
| A44 | 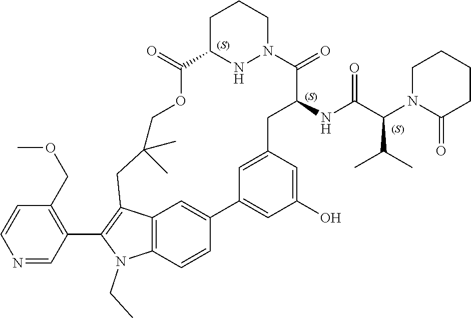 |
| A45 | 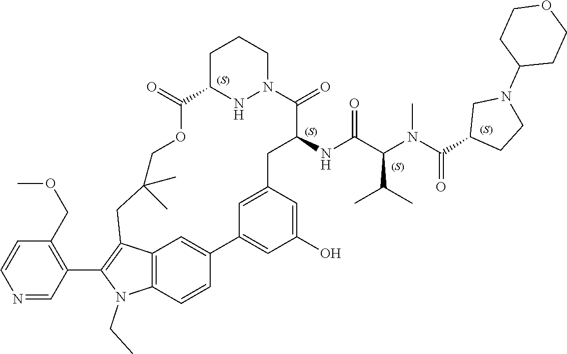 |
| A46 | 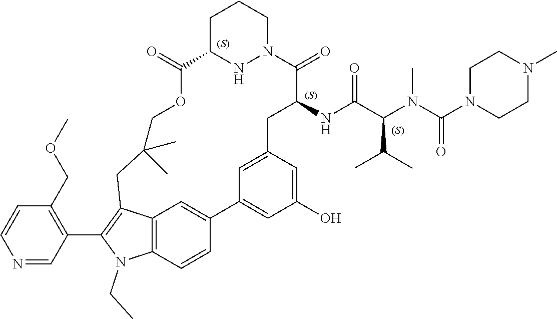 |
| A47 | 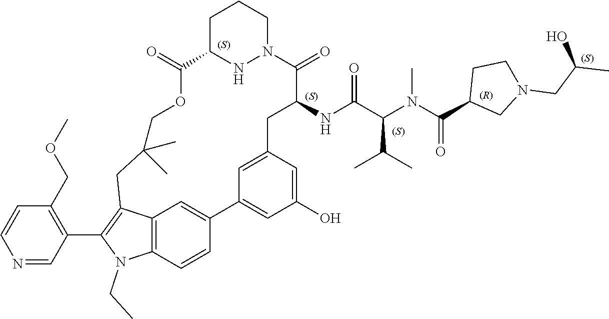 |
| A48 | 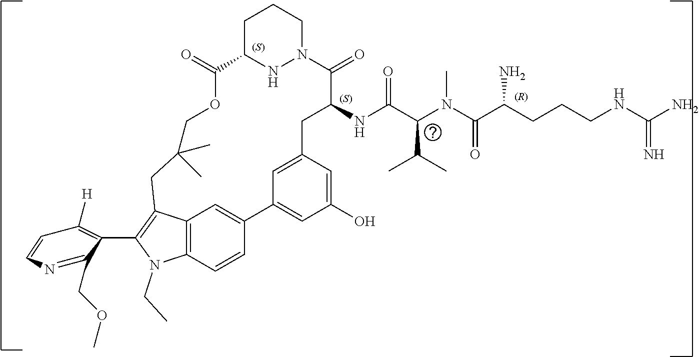 |
| A49 | 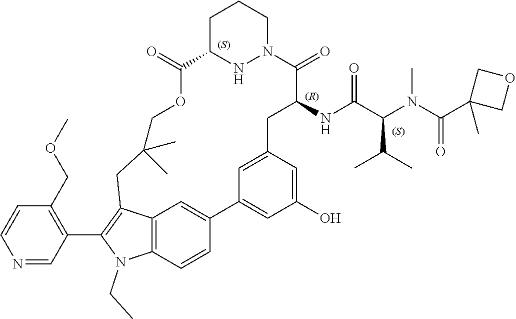 |
| A50 | 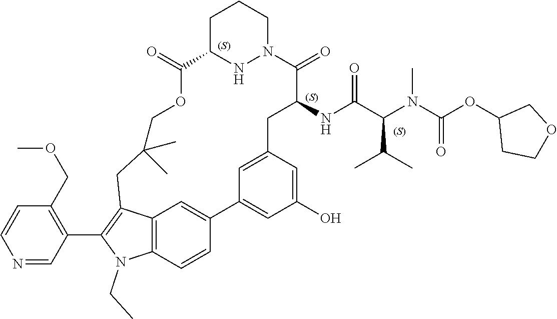 |
| A51 | 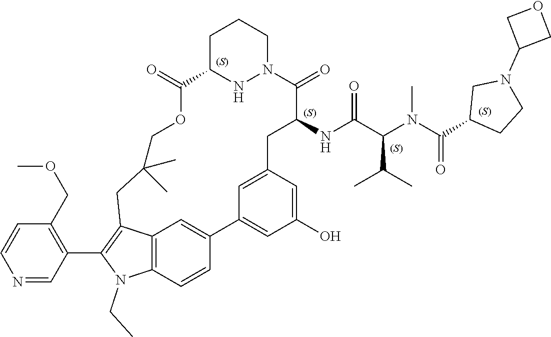 |
| A52 | 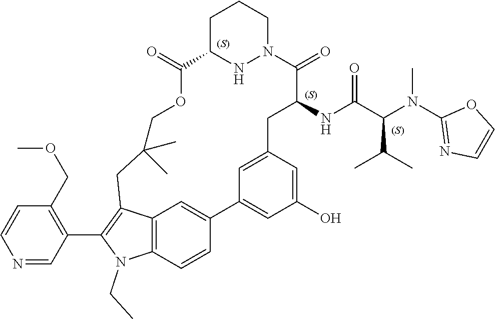 |
| A53 | 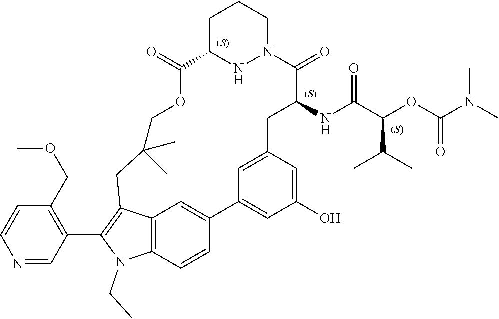 |
| A54 | 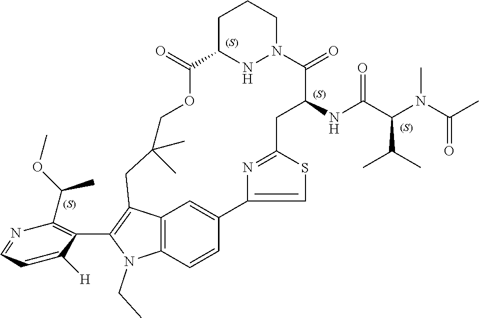 |
| A55 | 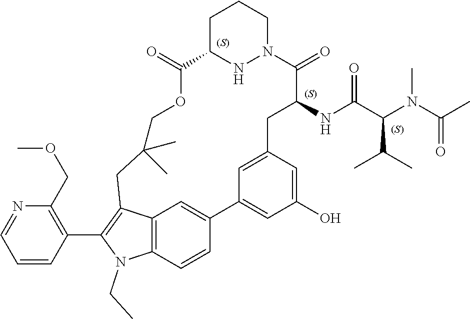 |
| A56 | 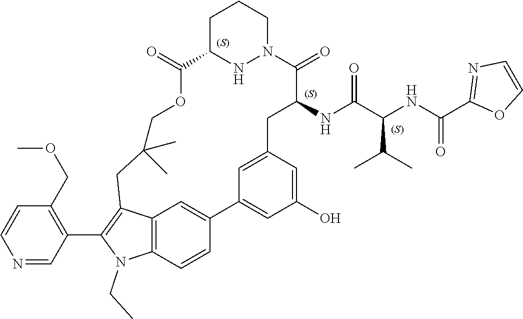 |
| A57 | 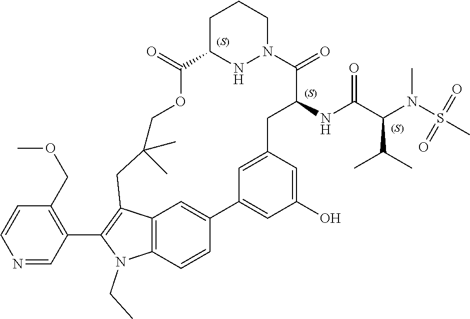 |
| A58 | 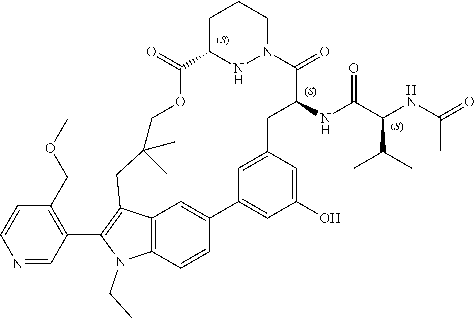 |
| A59 | 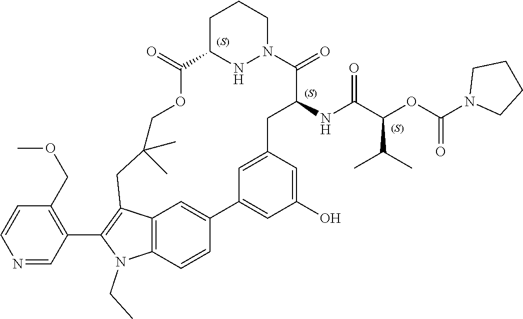 |
| A60 | 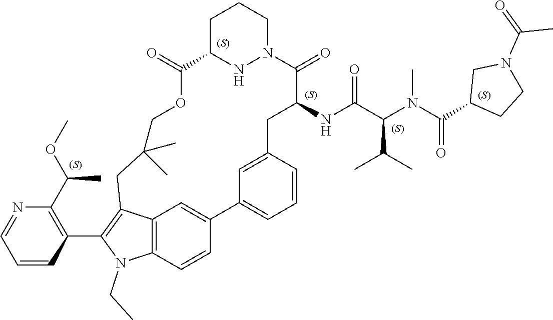 |
| A61 | 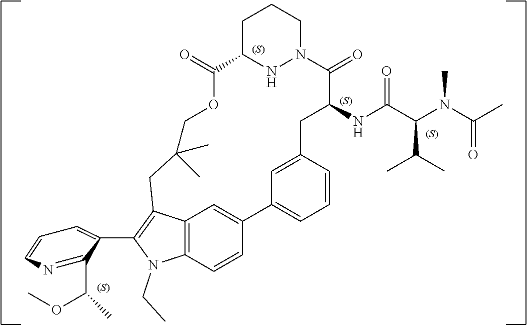 |
| A62 | 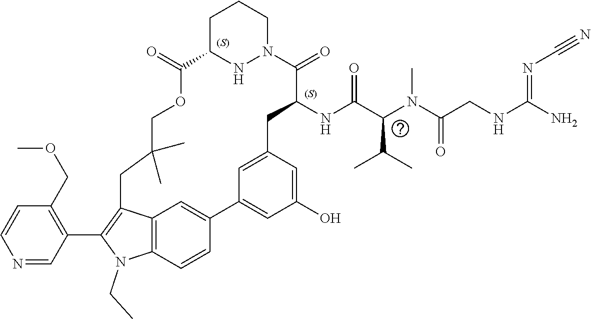 |
| A63 | 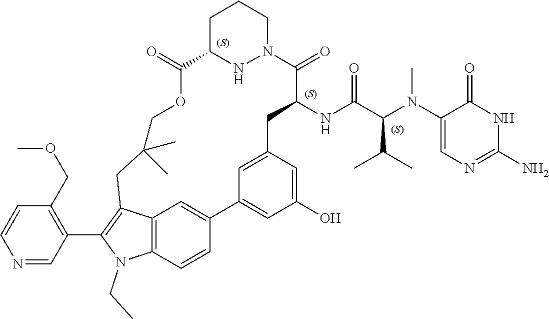 |
| A64 | 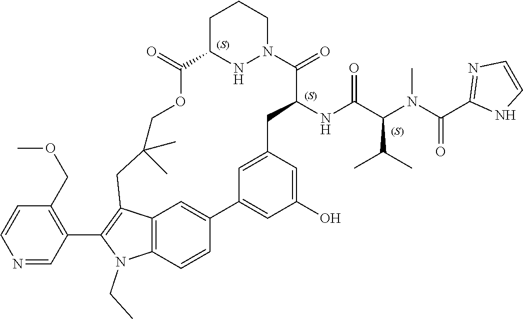 |
| A65 | 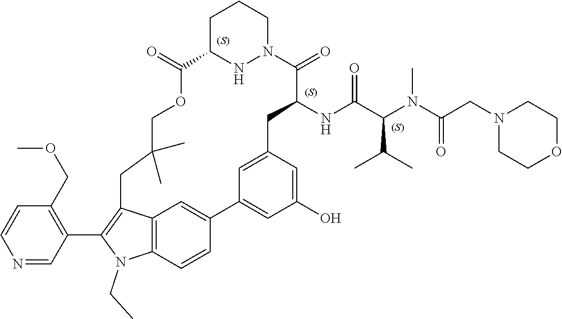 |
| A66 | 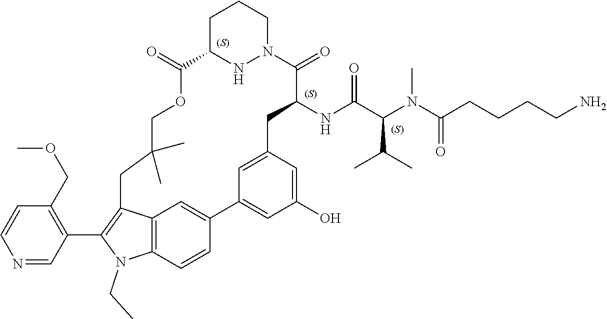 |
| A67 | 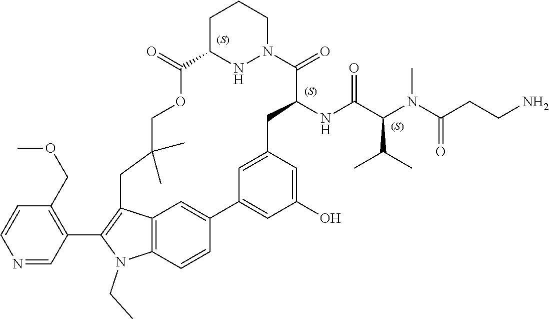 |
| A68 | 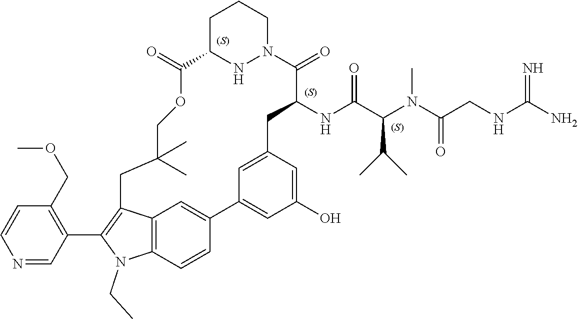 |
| A69 | 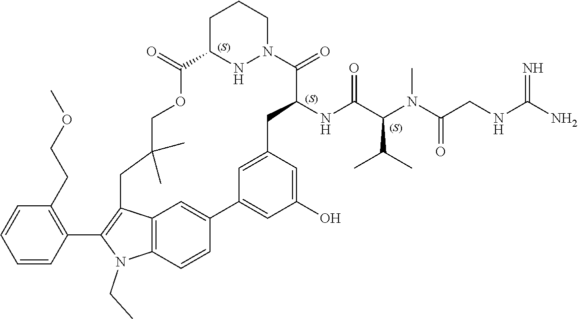 |
| A70 | 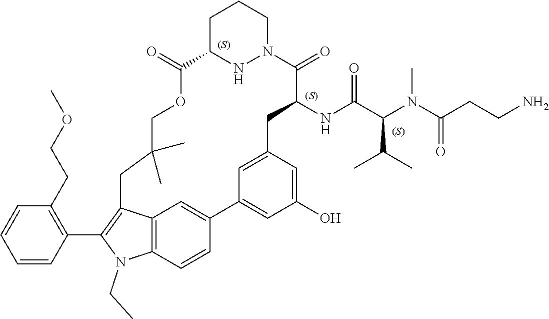 |
| A71 | 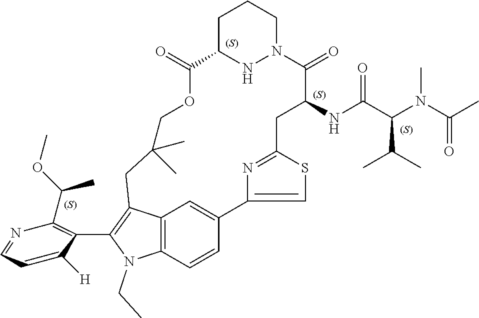 |
| A72 | 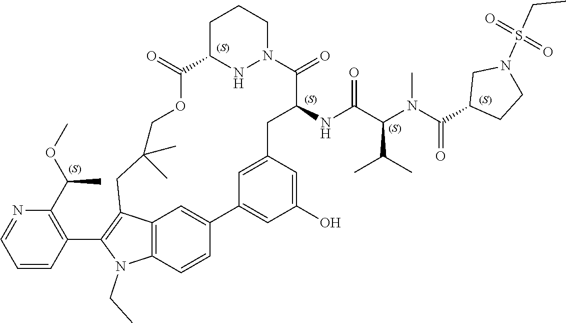 |
| A73 | 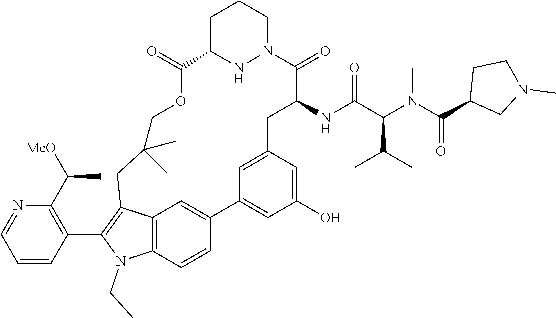 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| A74 | 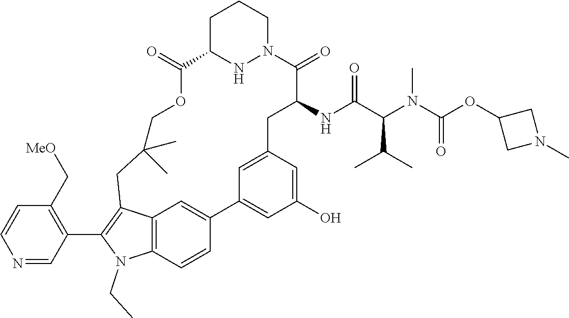 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| A75 | 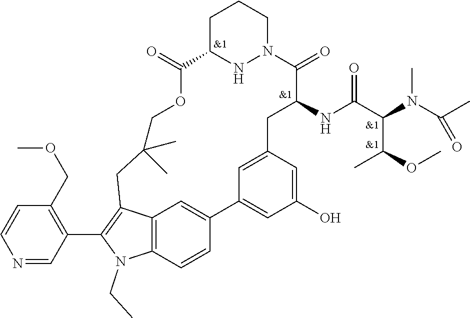 [SMILES: CCn1c(-c2cnccc2COC)c2c3cc(ccc31)-c1cc(O)cc(c1)C[C@H](NC(=O)[C@H]([C@H](C)OC)N(C)C(C)=O)C(=O)N1CCC[C@H](N1)C(=O)OCC(C)(C)C2] |
| A76 | 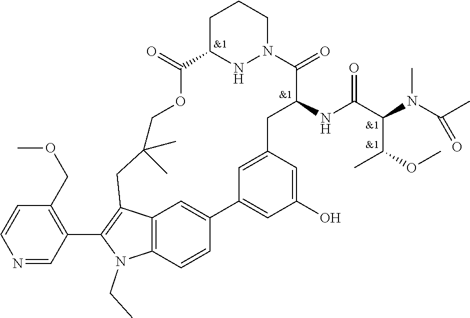 [SMILES: CCn1c(-c2cnccc2COC)c2c3cc(ccc31)-c1cc(O)cc(c1)C[C@H](NC(=O)[C@H]([C@@H](C)OC)N(C)C(C)=O)C(=O)N1CCC[C@H](N1)C(=O)OCC(C)(C)C2] |
| A77 | 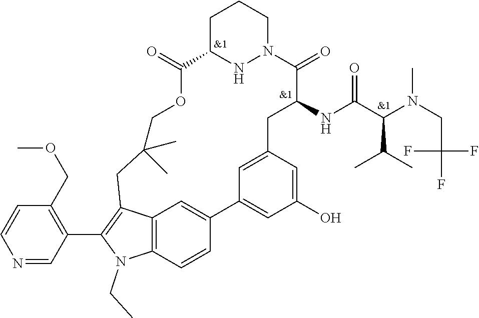 [SMILES: CCn1c(-c2cnccc2COC)c2c3cc(ccc31)-c1cc(O)cc(c1)C[C@H](NC(=O)[C@H](C(C)C)N(C)CC(F)(F)F)C(=O)N1CCC[C@H](N1)C(=O)OCC(C)(C)C2] |
| A78 | 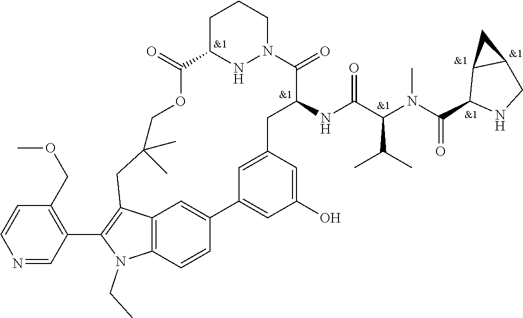 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NC[C@@H]3C[C@@H]31)C(=O)N1CCC[C@H](N1)C(=O)OCC(C)(C)C2] |
| A79 | 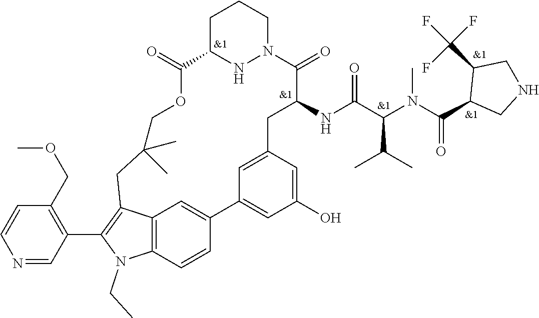 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1C(F)(F)F)C(=O)N1CCC[C@H](N1)C(=O)OCC(C)(C)C2] |
| A80 | 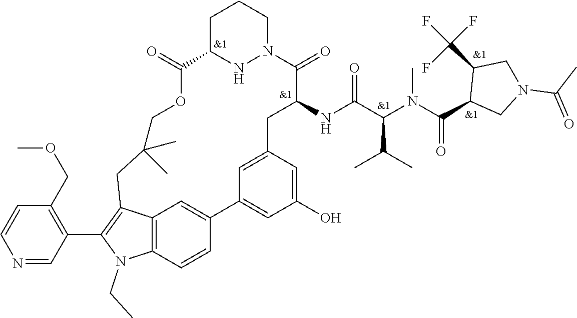 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(C)=O)C[C@@H]1C(F)(F)F)C(=O)N1CCC[C@H](N1)C(=O)OCC(C)(C)C2] |
| A81 | 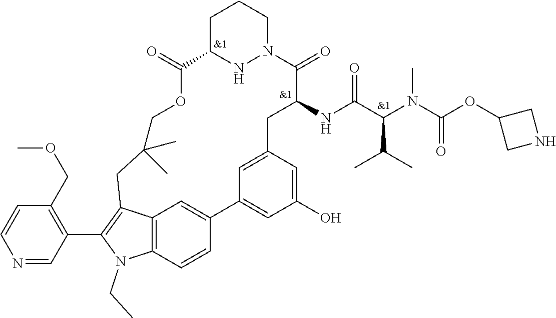 [SMILES: CCn1c(-c2cnccc2COC)c2c3cc(ccc31)-c1cc(O)cc(c1)C[C@H](NC(=O)[C@H](C(C)C)N(C)C(=O)OC1CNC1)C(=O)N1CCC[C@H](N1)C(=O)OCC(C)(C)C2] |
| A82 | 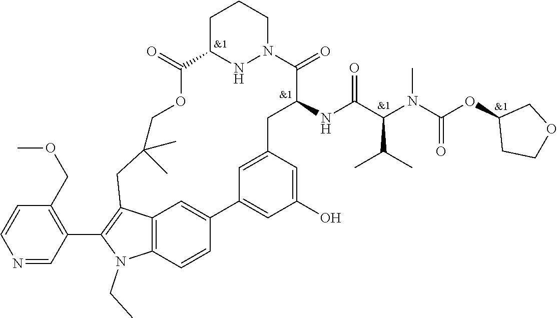 [SMILES: CCn1c(-c2cnccc2COC)c2c3cc(ccc31)-c1cc(O)cc(c1)C[C@H](NC(=O)[C@H](C(C)C)N(C)C(=O)O[C@@H]1CCOC1)C(=O)N1CCC[C@H](N1)C(=O)OCC(C)(C)C2] |
| A83 | 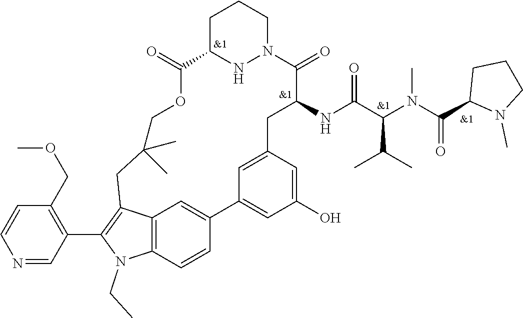 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCN1C)C(=O)N1CCC[C@H](N1)C(=O)OCC(C)(C)C2] |
| A84 | 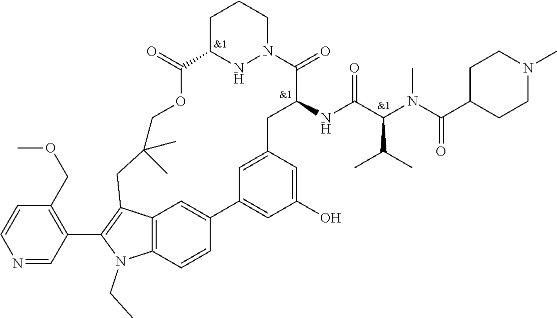 [SMILES: CCn1c(-c2cnccc2COC)c2c3cc(ccc31)-c1cc(O)cc(c1)C[C@H](NC(=O)[C@H](C(C)C)N(C)C(=O)C1CCN(C)CC1)C(=O)N1CCC[C@H](N1)C(=O)OCC(C)(C)C2] |
| A85 | 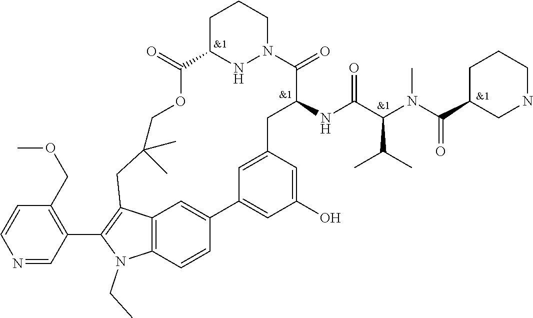 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCNC1)C(=O)N1CCC[C@H](N1)C(=O)OCC(C)(C)C2] |
| A86 | 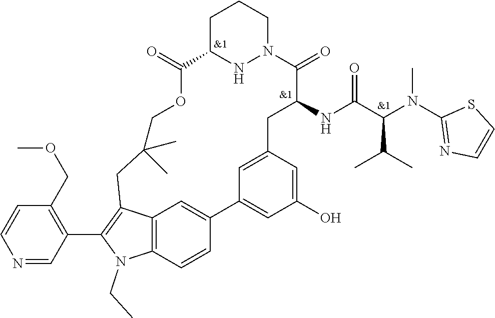 [SMILES: CCn1c(-c2cnccc2COC)c2c3cc(ccc31)-c1cc(O)cc(c1)C[C@H](NC(=O)[C@H](C(C)C)N(C)c1nccs1)C(=O)N1CCC[C@H](N1)C(=O)OCC(C)(C)C2] |
| A87 | 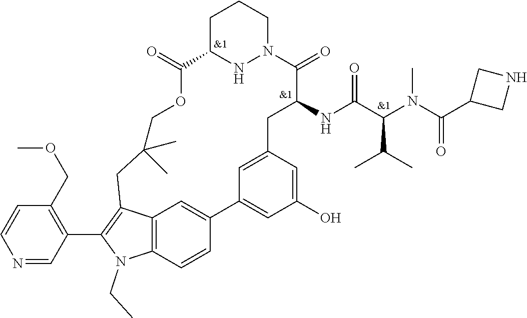 [SMILES: CCn1c(-c2cnccc2COC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| A88 | 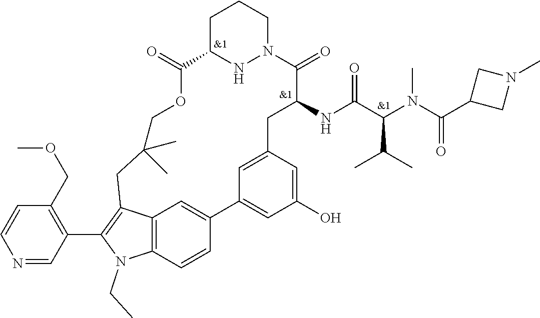 [SMILES: CCn1c(-c2cnccc2COC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| A89 | 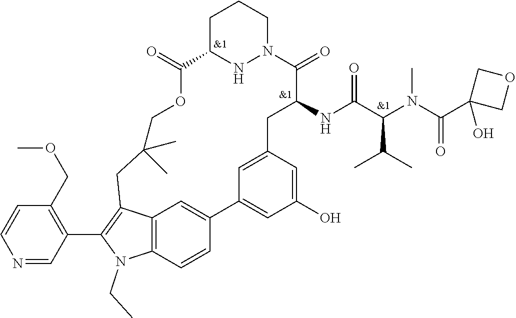 [SMILES: CCn1c(-c2cnccc2COC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| A90 | 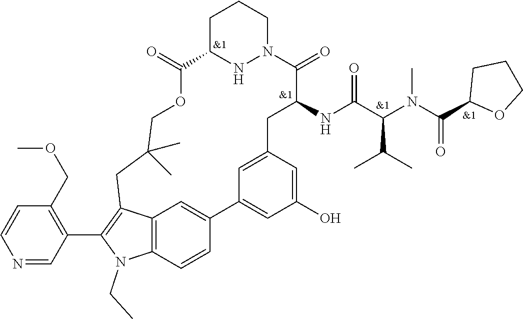 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCO1)C(=O)N1CCC[C@H](N1)C(=O)OCC(C)(C)C2] |
| A91 | 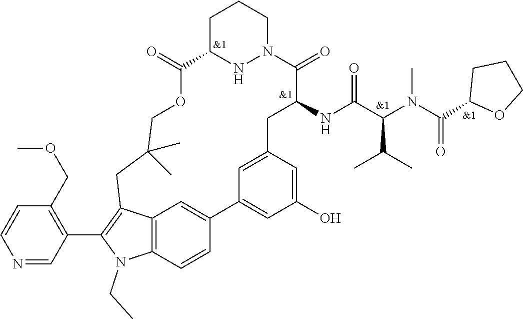 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCO1)C(=O)N1CCC[C@H](N1)C(=O)OCC(C)(C)C2] |
| A92 | 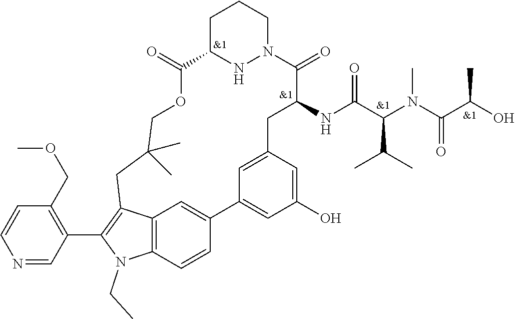 [SMILES: CCn1c(-c2cnccc2COC)c2c3cc(ccc31)-c1cc(O)cc(c1)C[C@H](NC(=O)[C@H](C(C)C)N(C)C(=O)[C@@H](C)O)C(=O)N1CCC[C@H](N1)C(=O)OCC(C)(C)C2] |
| A93 | 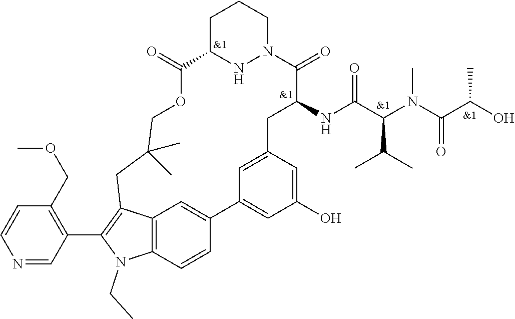 [SMILES: CCn1c(-c2cnccc2COC)c2c3cc(ccc31)-c1cc(O)cc(c1)C[C@H](NC(=O)[C@H](C(C)C)N(C)C(=O)[C@H](C)O)C(=O)N1CCC[C@H](N1)C(=O)OCC(C)(C)C2] |
| A94 | 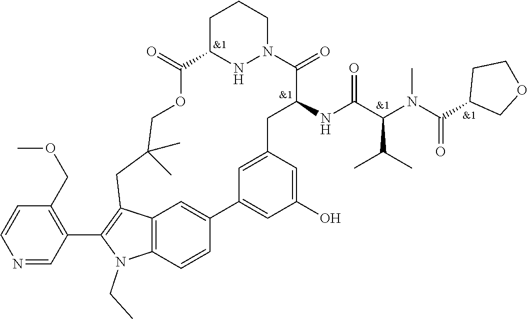 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCOC1)C(=O)N1CCC[C@H](N1)C(=O)OCC(C)(C)C2] |
| A95 | 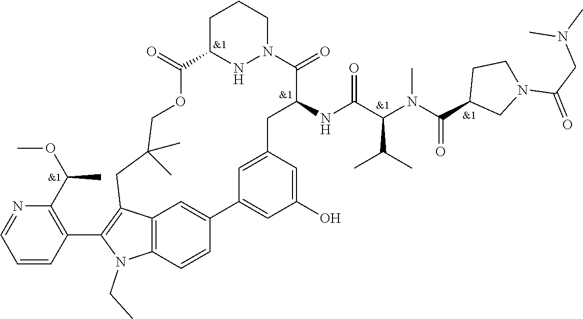 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| A96 | 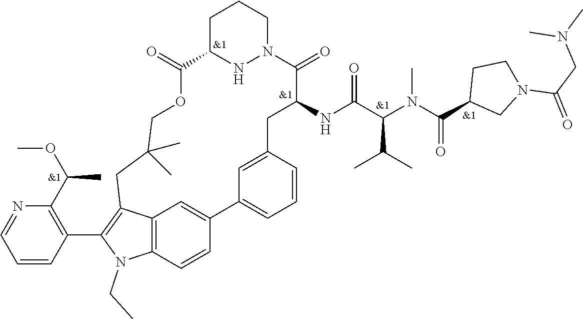 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| A97 | 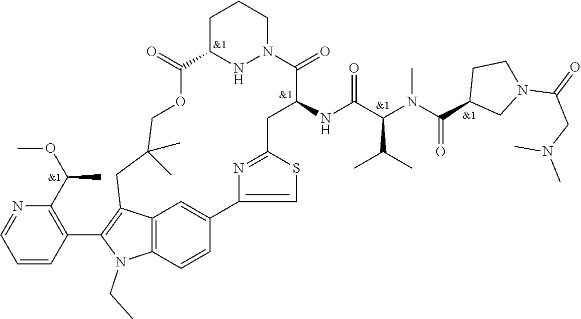 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| A98 | 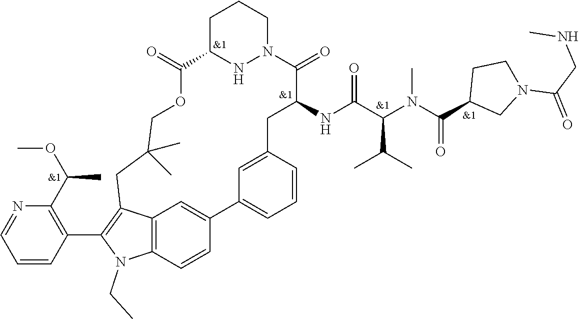 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| A99 | 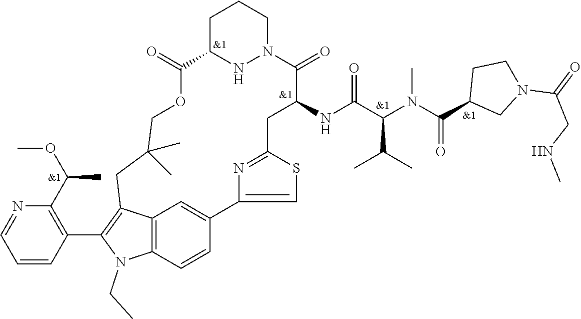 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| A100 | 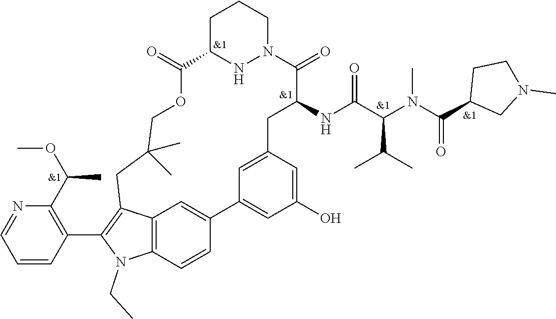 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| A101 | 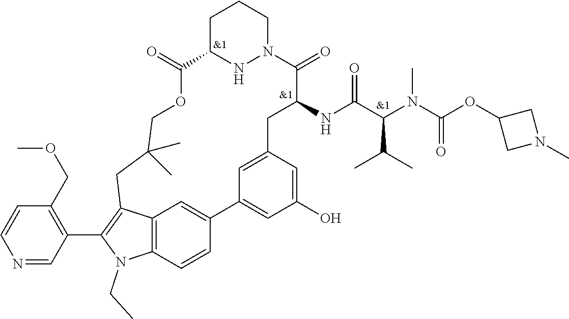 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| A102 | 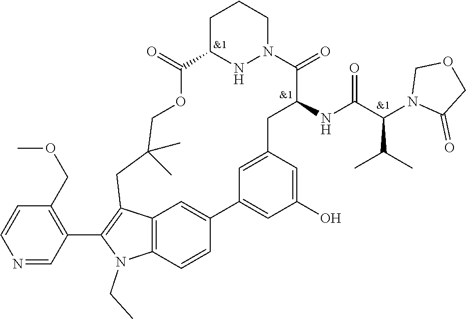 [SMILES: CCn1c(-c2cnccc2COC)c2c3cc(ccc31)-c1cc(O)cc(c1)C[C@H](NC(=O)[C@H](C(C)C)N1COCC1=O)C(=O)N1CCC[C@H](N1)C(=O)OCC(C)(C)C2] |
| A103 | 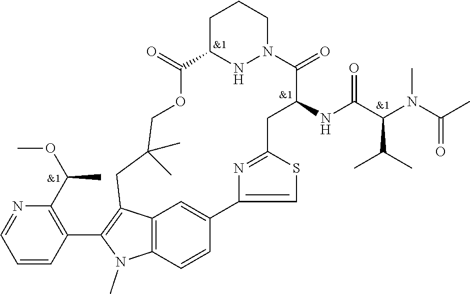 [SMILES: CO[C@@H](C)c1ncccc1-c1c2c3cc(ccc3n1C)-c1csc(n1)C[C@H](NC(=O)[C@H](C(C)C)N(C)C(C)=O)C(=O)N1CCC[C@H](N1)C(=O)OCC(C)(C)C2] |
| A104 | 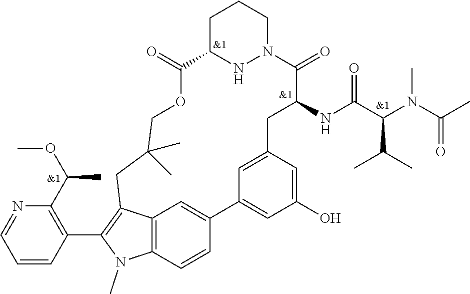 [SMILES: CO[C@@H](C)c1ncccc1-c1c2c3cc(ccc3n1C)-c1cc(O)cc(c1)C[C@H](NC(=O)[C@H](C(C)C)N(C)C(C)=O)C(=O)N1CCC[C@H](N1)C(=O)OCC(C)(C)C2] |
| A105 | 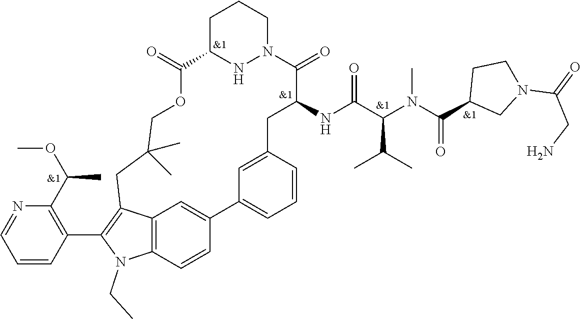 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| A106 | 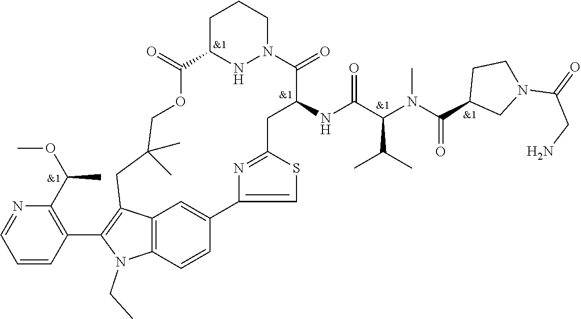 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| A107 | 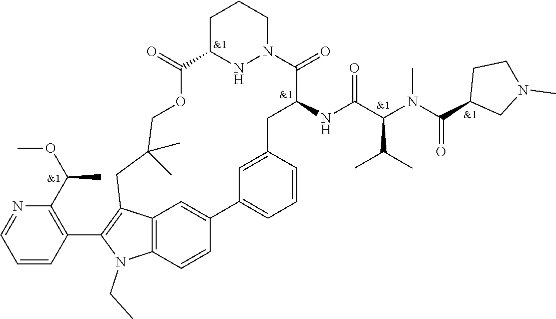 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| A108 | 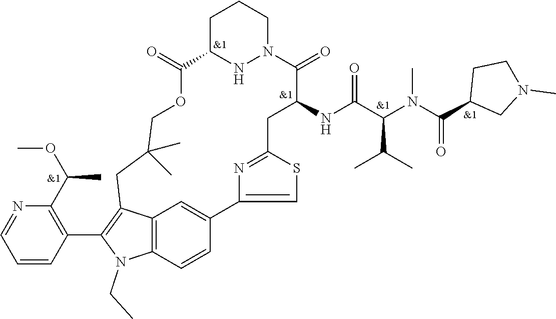 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C)C1)C(=O)N1CCC[C@H](N1)C(=O)OCC(C)(C)C2] |
| A109 | 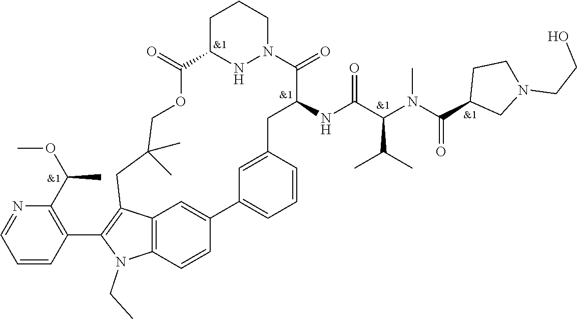 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| A110 | 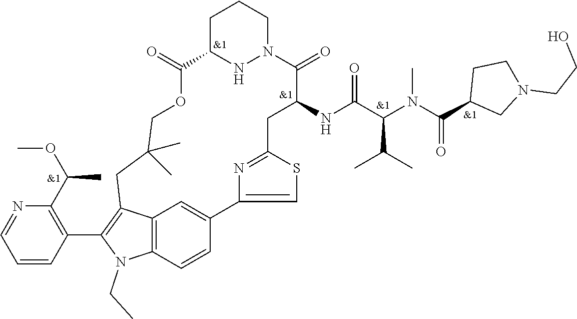 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(CCO)C1)C(=O)N1CCC[C@H](N1)C(=O)OCC(C)(C)C2] |
| A111 | 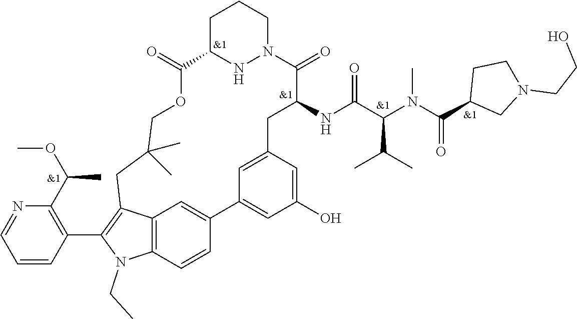 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| A112 | 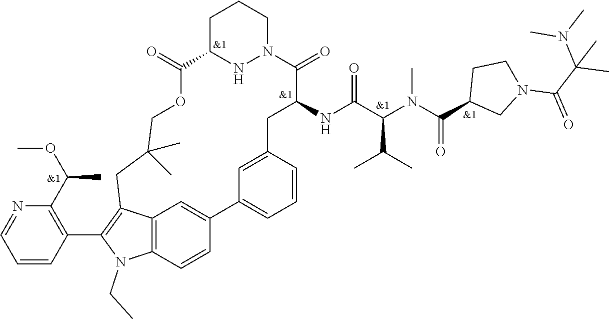 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A113 | 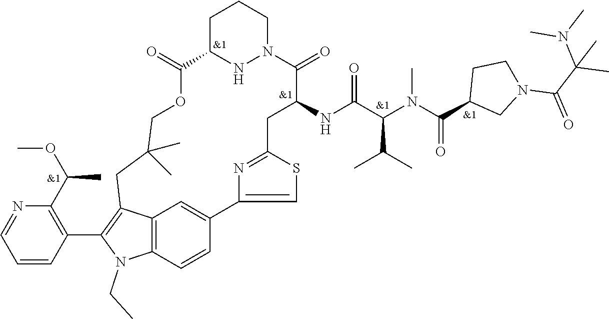 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A114 | 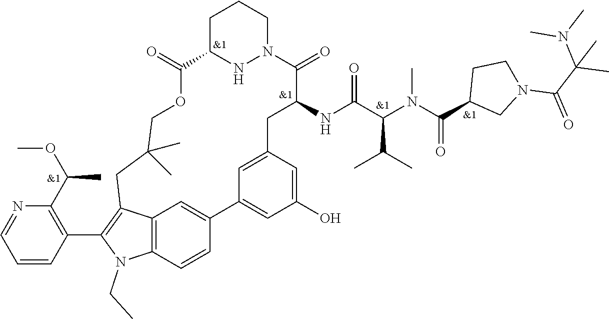 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A115 | 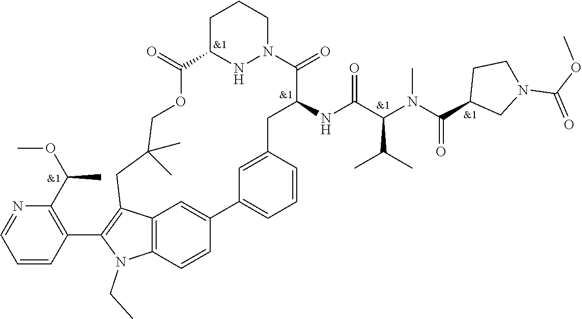 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| A116 | 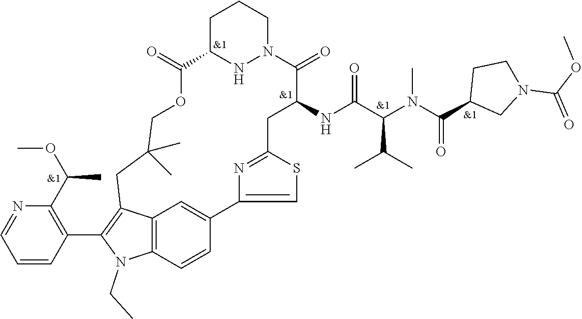 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| A117 | 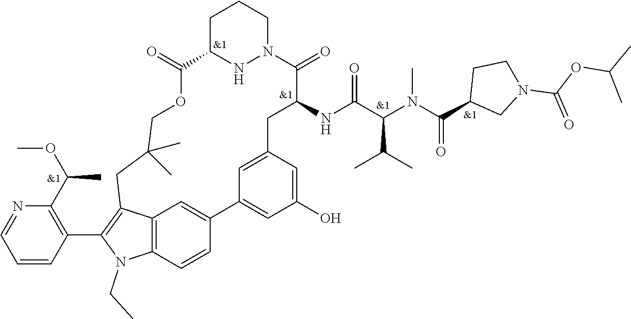 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)OC(C)C)C1)C(=O)N1CCC[C@H](N1)C(=O)OCC(C)(C)C2] |
| A118 | 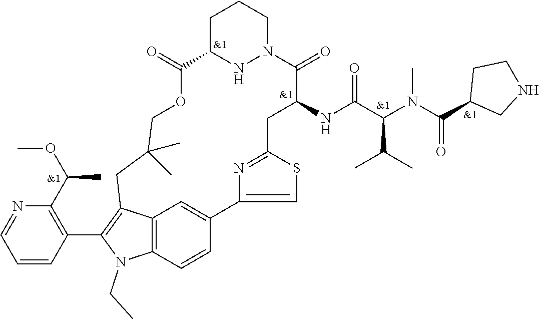 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCNC1)C(=O)N1CCC[C@H](N1)C(=O)OCC(C)(C)C2] |
| A119 | 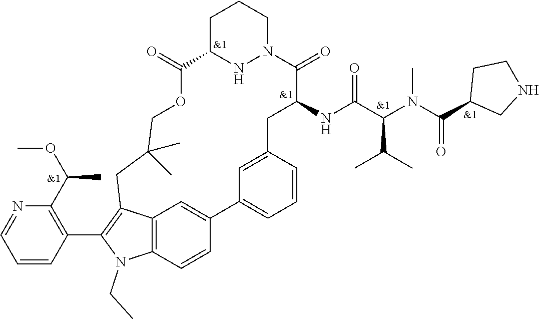 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCNC1)C(=O)N1CCC[C@H](N1)C(=O)OCC(C)(C)C2] |
| A120 | 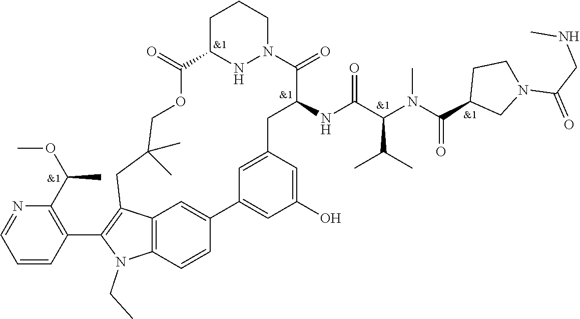 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| A121 | 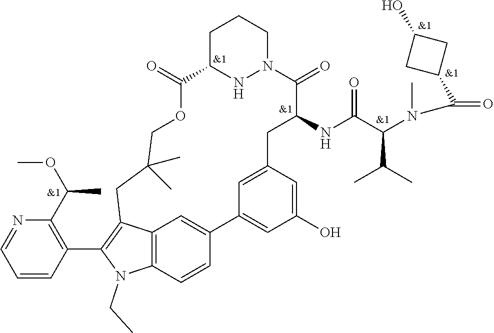 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H](O)C1)C(=O)N1CCC[C@H](N1)C(=O)OCC(C)(C)C2] |
| A122 | 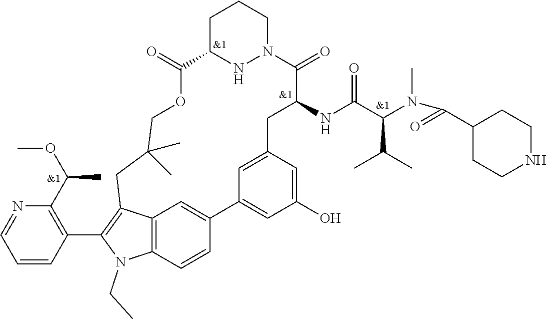 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNCC1)C(=O)N1CCC[C@H](N1)C(=O)OCC(C)(C)C2] |
| A123 | 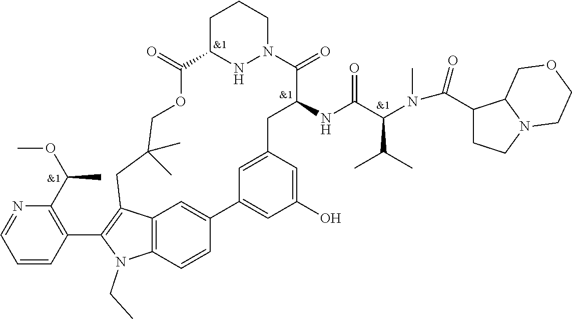 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCN3CCOCC13)C(=O)N1CCC[C@H](N1)C(=O)OCC(C)(C)C2] |
| A124 | 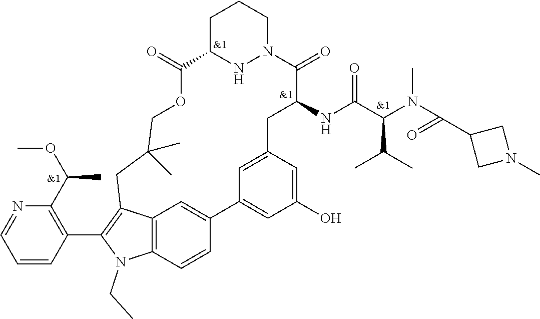 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| A125 | 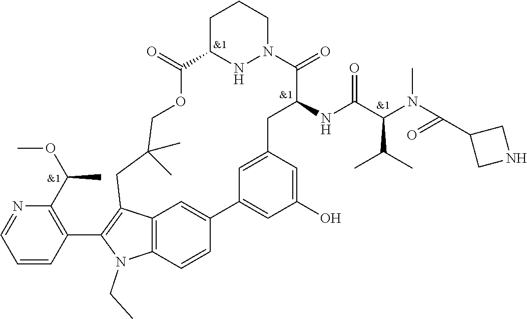 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| A126 | 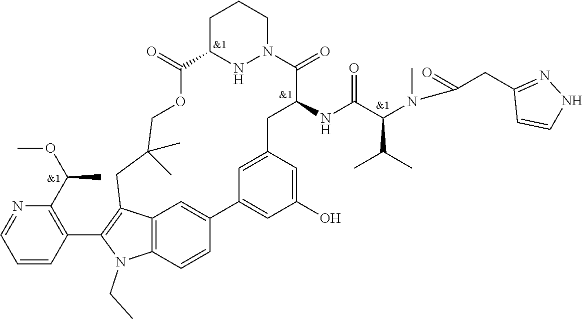 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1cc[nH]n1)C(=O)N1CCC[C@H](N1)C(=O)OCC(C)(C)C2] |
| A127 | 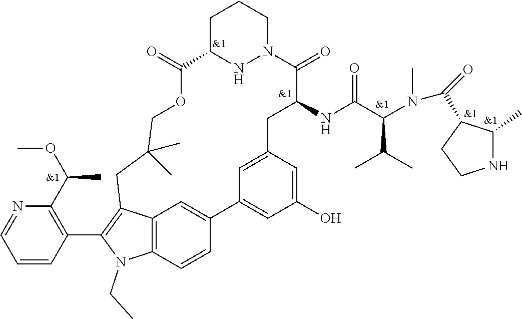 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN[C@H]1C)C(=O)N1CCC[C@H](N1)C(=O)OCC(C)(C)C2] |
| A128 | 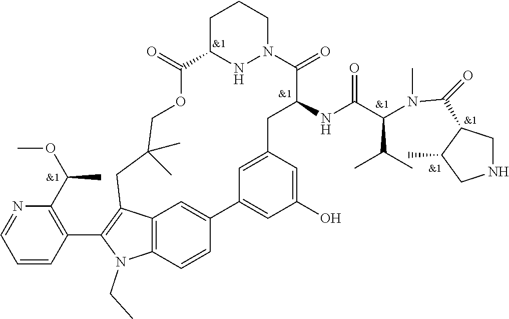 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1C)C(=O)N1CCC[C@H](N1)C(=O)OCC(C)(C)C2] |
| A129 | 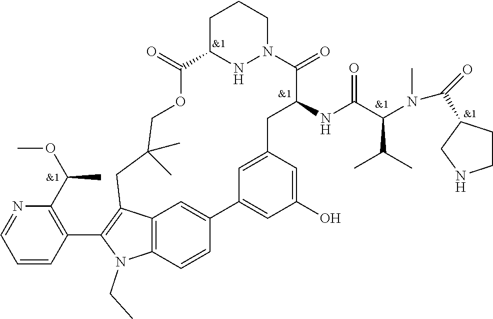 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCNC1)C(=O)N1CCC[C@H](N1)C(=O)OCC(C)(C)C2] |
| A130 | 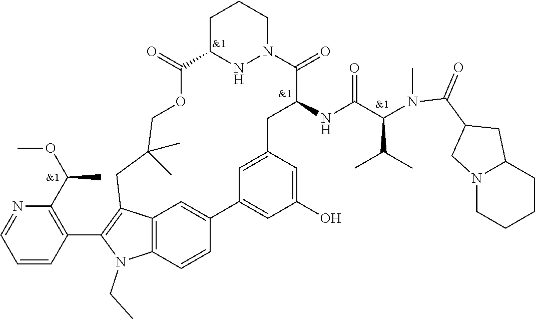 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CCCCN3C1)C(=O)N1CCC[C@H](N1)C(=O)OCC(C)(C)C2] |
| A131 | 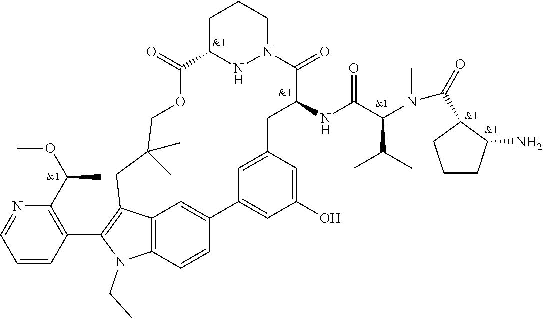 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCC[C@H]1N)C(=O)N1CCC[C@H](N1)C(=O)OCC(C)(C)C2] |
| A132 | 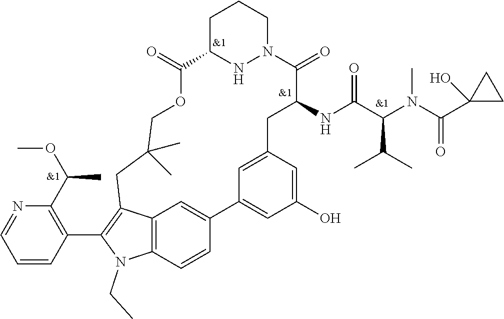 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)CC1)C(=O)N1CCC[C@H](N1)C(=O)OCC(C)(C)C2] |
| A133 | 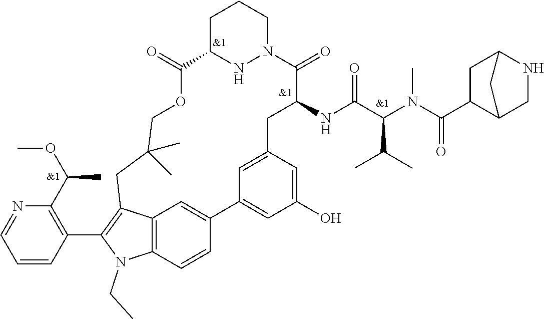 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CC1CN3)C(=O)N1CCC[C@H](N1)C(=O)OCC(C)(C)C2] |
| A134 | 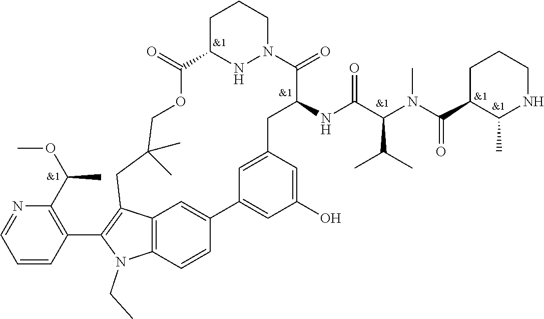 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCCN[C@@H]1C)C(=O)N1CCC[C@H](N1)C(=O)OCC(C)(C)C2] |
| A135 | 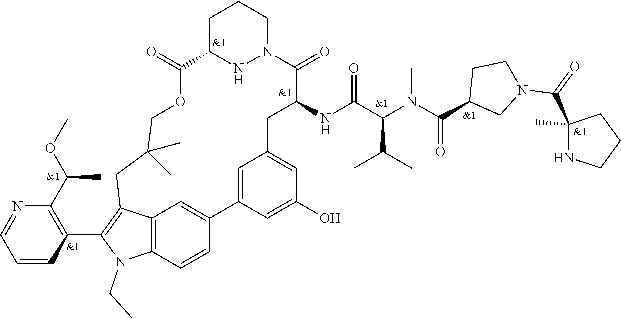 |
| A136 | 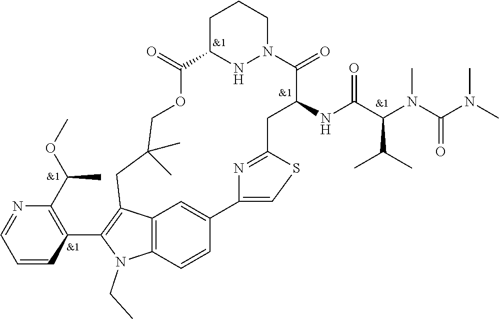 |
| A137 | 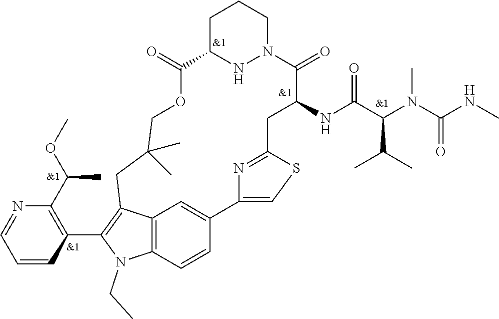 |
| A138 | 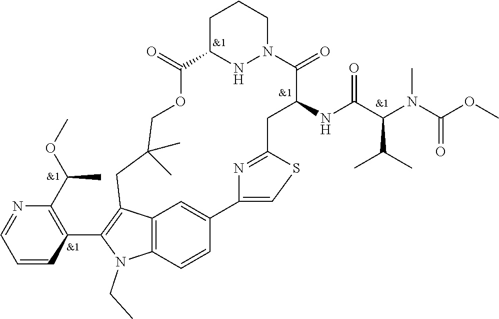 |
| A139 | 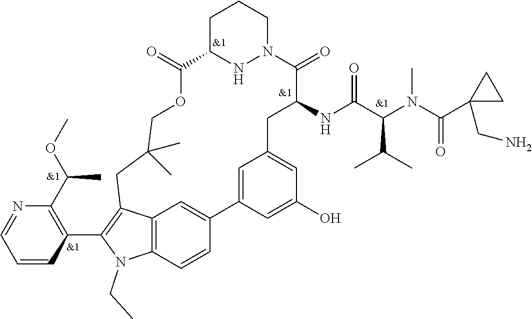 |
| A140 | 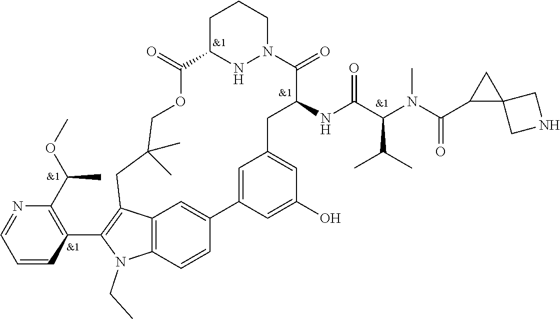 |
| A141 | 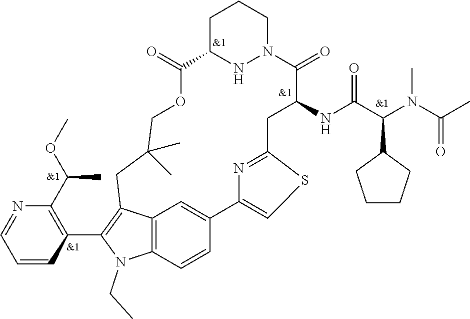 |
| A142 | 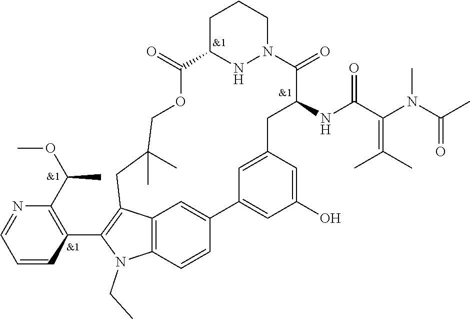 |
| A143 | 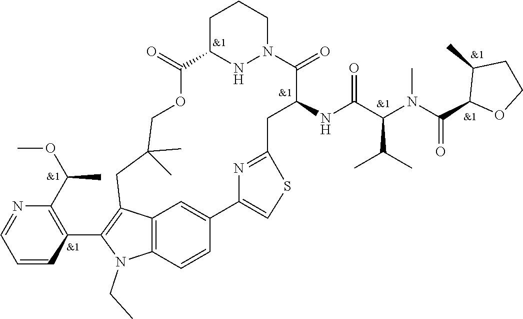 |
| A144 | 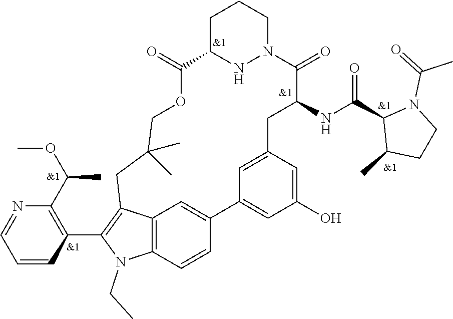 |
| A145 | 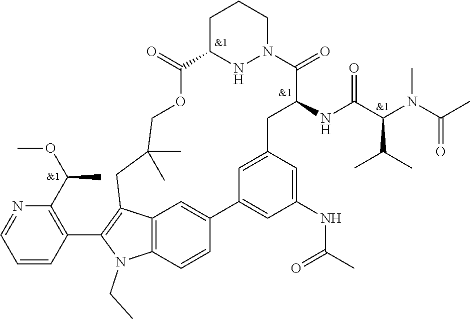 [SMILES: CCn1c(-c2cccnc2[C@H](C)OC)c2c3cc(ccc31)-c1cc(cc(NC(C)=O)c1)C[C@H](NC(=O)[C@H](C(C)C)N(C)C(C)=O)C(=O)N1CCC[C@H](N1)C(=O)OCC(C)(C)C2] |
| A146 | 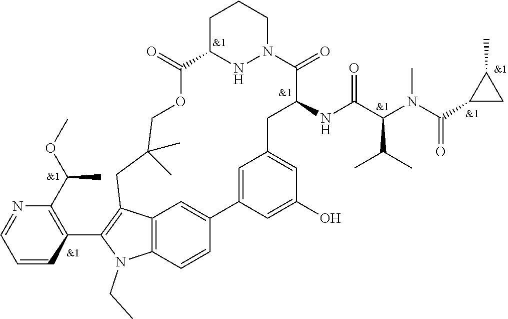 |
| A147 | 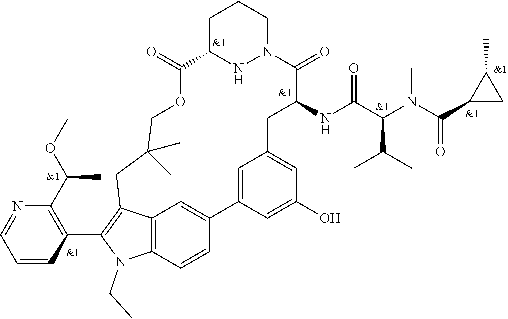 |
| A148 | 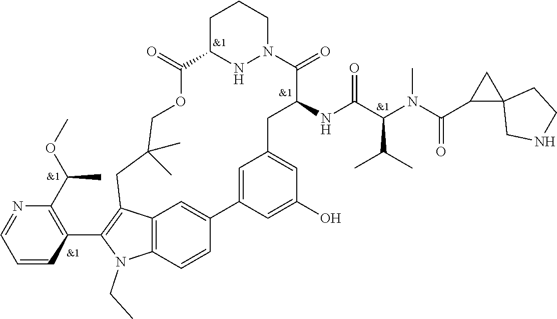 |
| A149 | 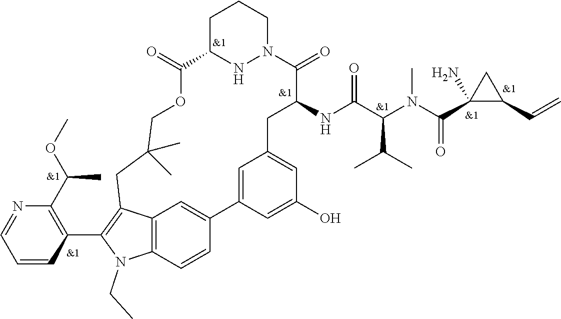 |
| A150 | 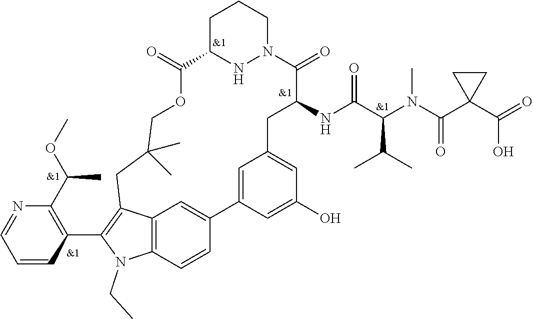 |
| A151 | 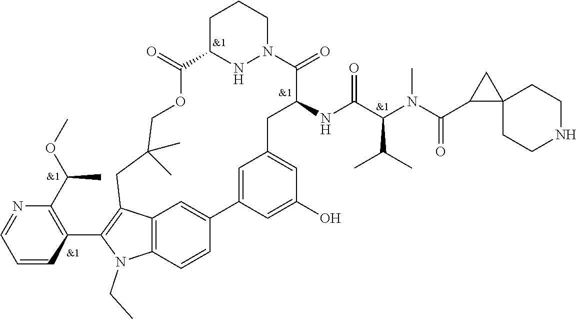 |
| A152 | 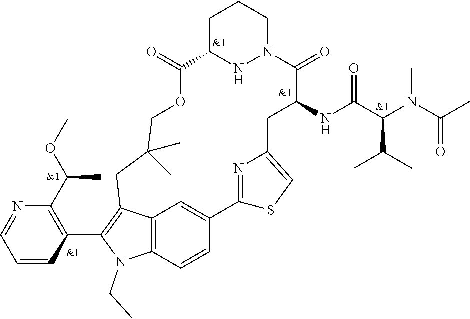 |
| A153 | 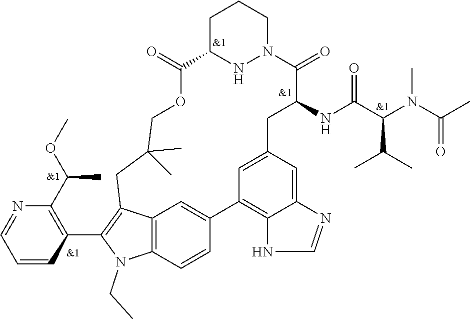 |
| A154 | 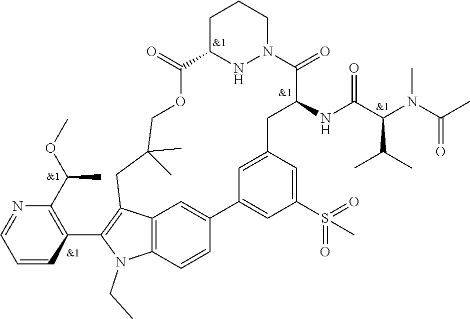 |
| A155 | 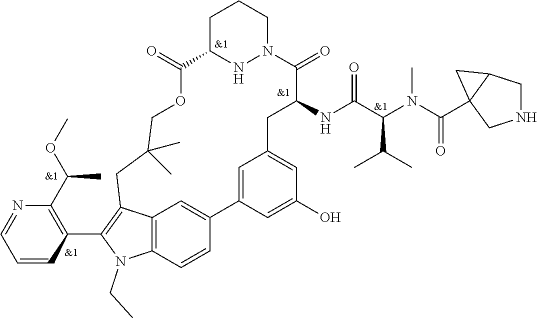 |
| A156 | 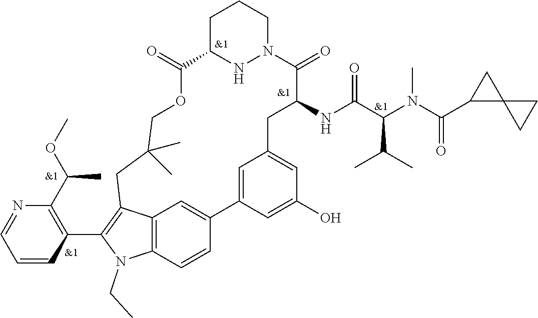 |
| A157 | 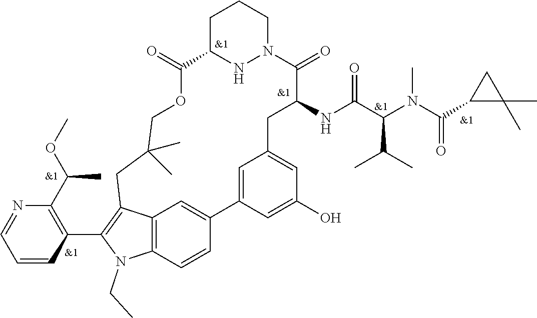 |
| A158 | 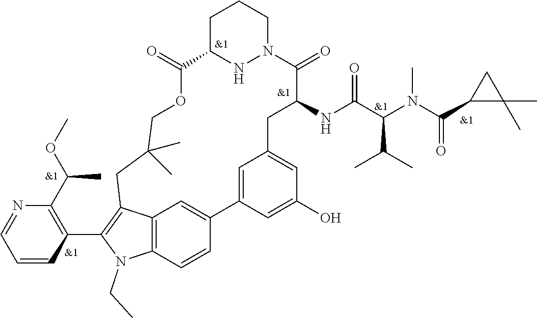 |
| A159 | 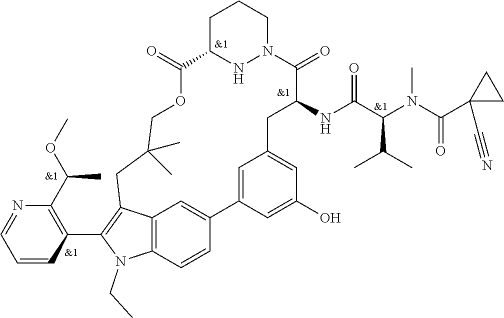 |
| A160 | 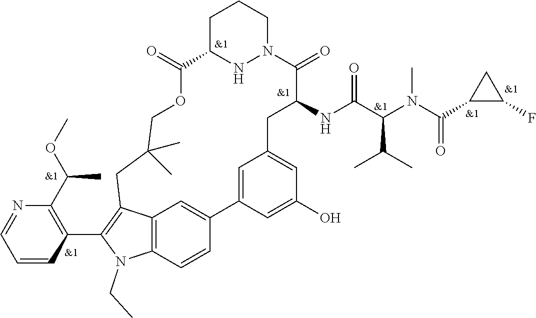 |
| A161 | 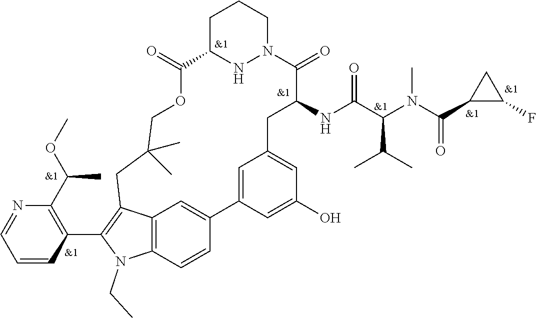 |
| A162 | 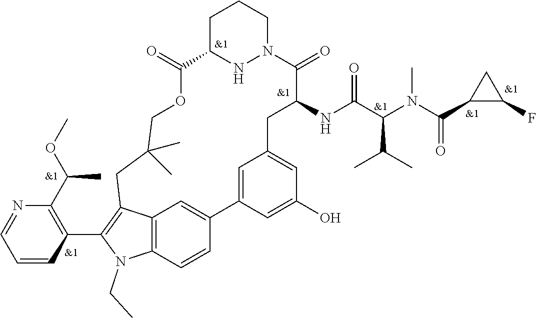 |
| A163 | 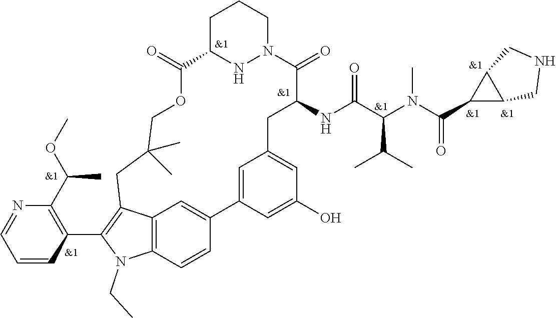 |
| A164 | 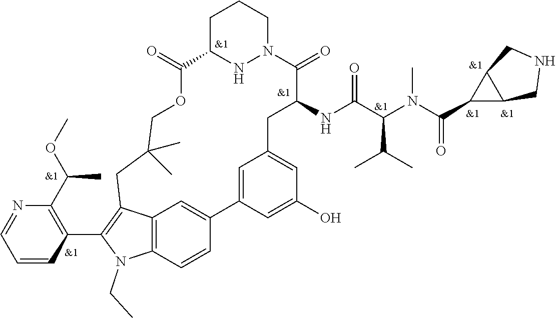 |
| A165 | 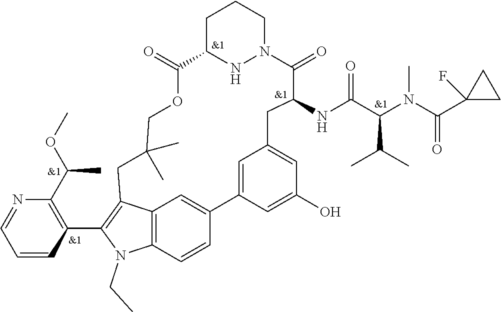 |
| A166 | 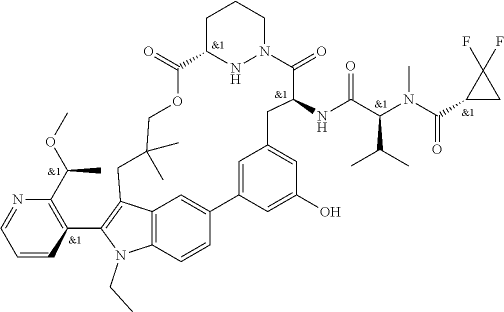 |
| A167 | 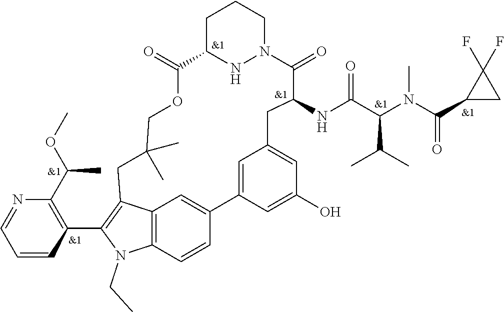 |
| A168 | 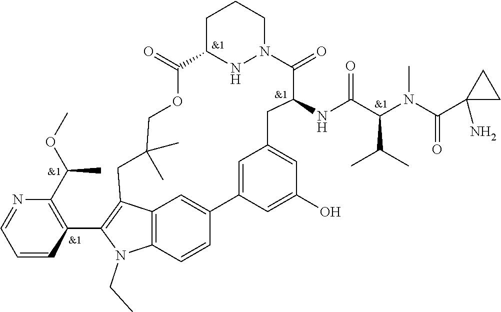 |
| A169 | 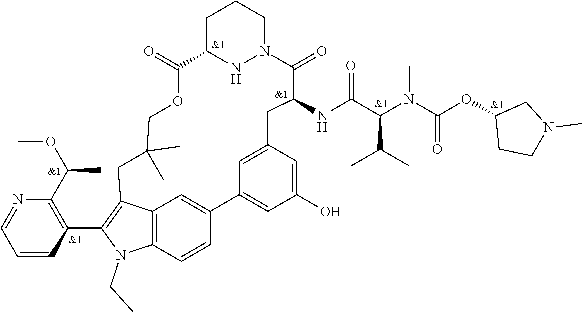 |
| A170 | 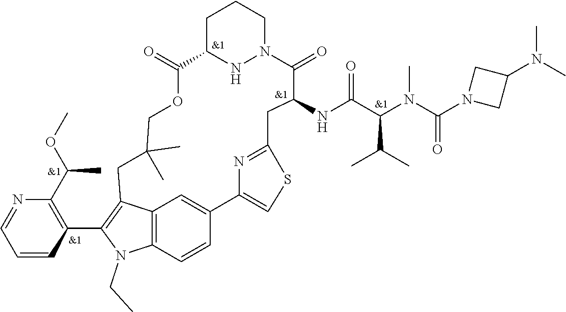 |
| A171 | 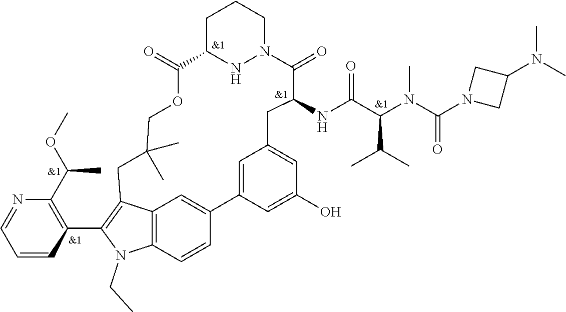 |
| A172 | 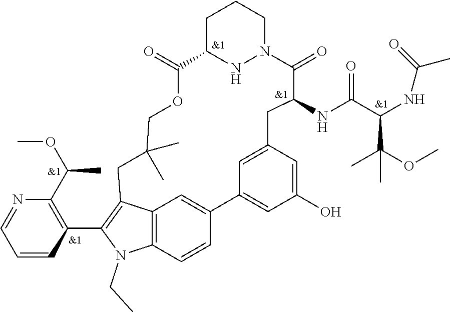 |
| A173 | 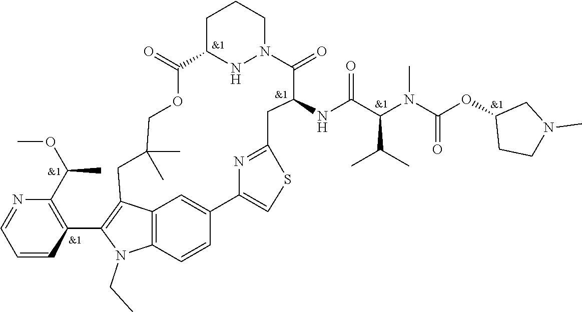 |
| A174 | 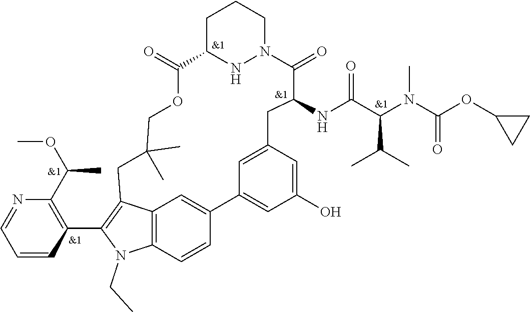 |
| A175 | 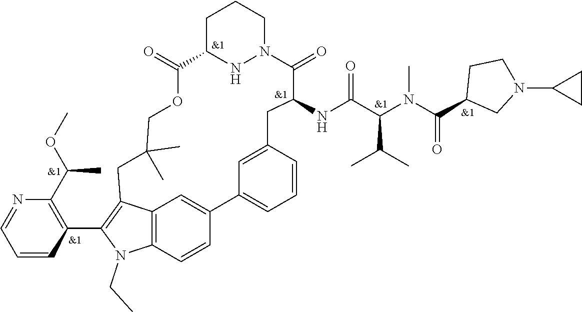 |
| A176 | 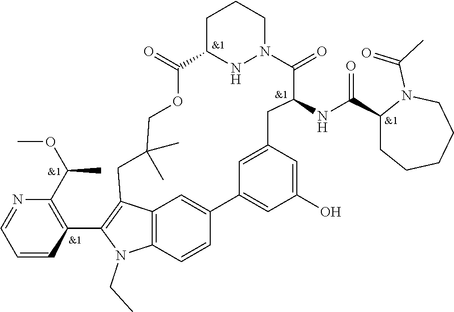 |
| A177 | 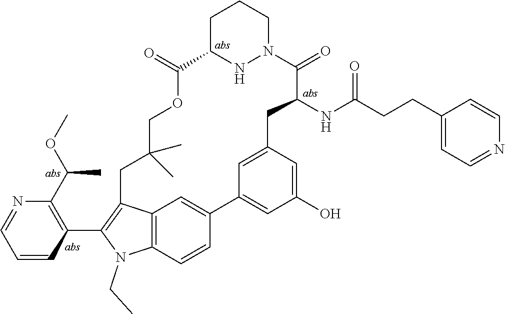 |
| A178 | 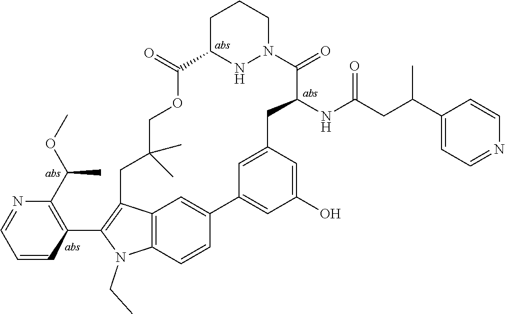 |
| A179 | 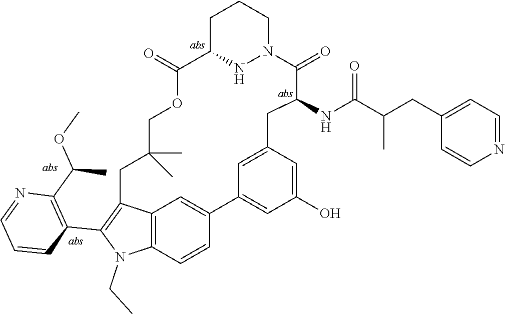 |
| A180 | 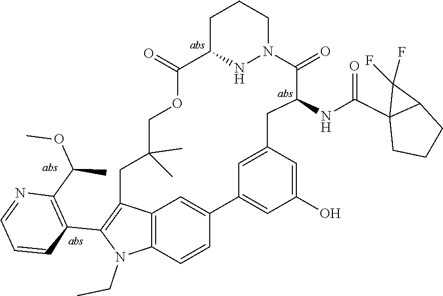 |
| A181 | 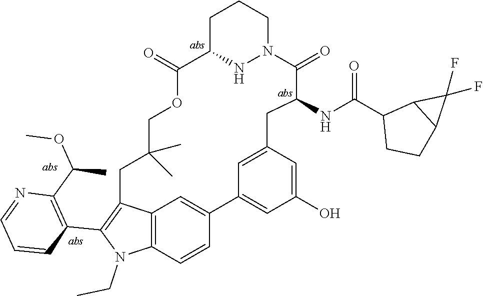 |
| A182 | 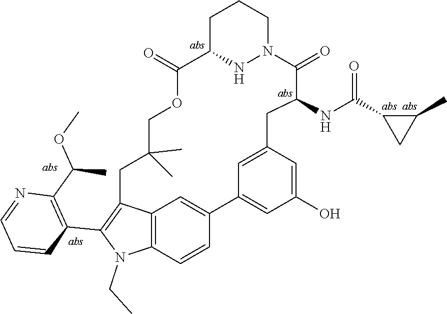 |
| A183 | 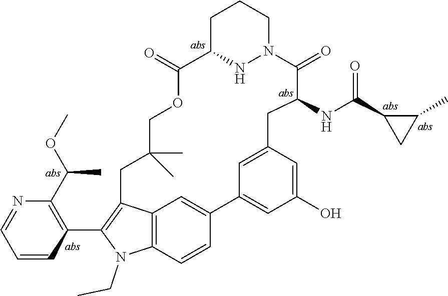 |
| A184 | 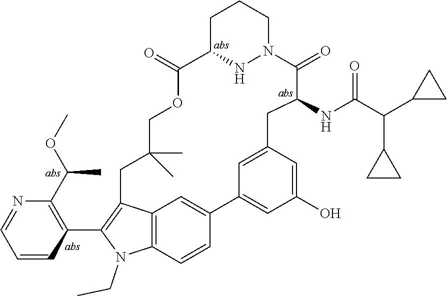 |
| A185 | 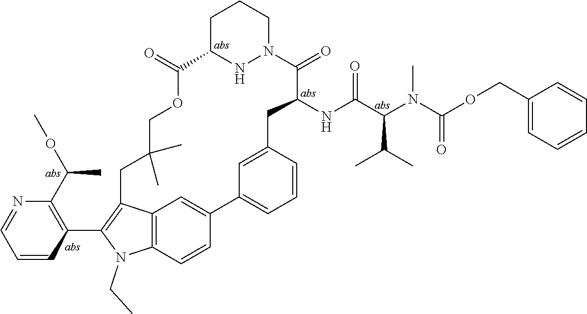 |
| A186 | 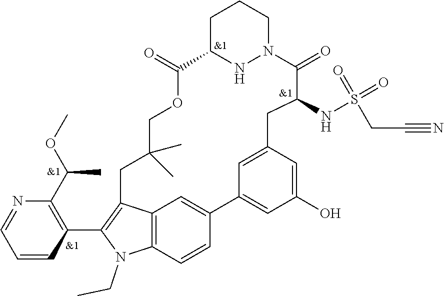 |
| A187 | 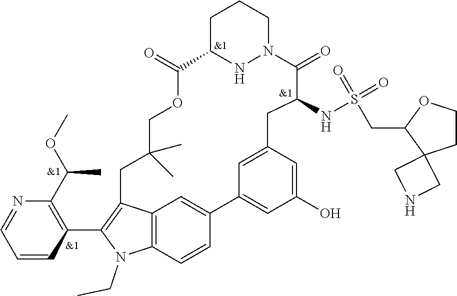 |
| A188 | 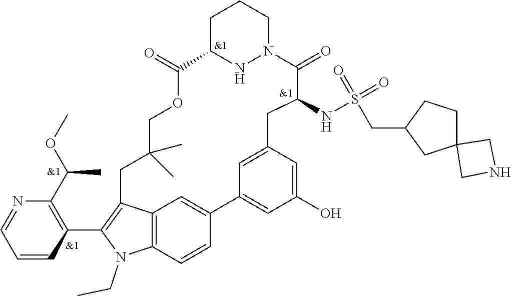 |
| A189 | 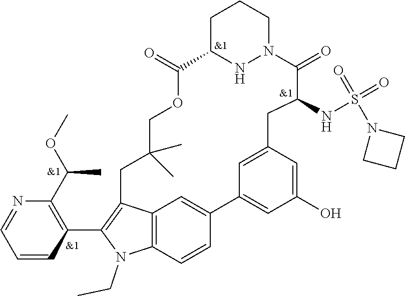 |
| A190 | 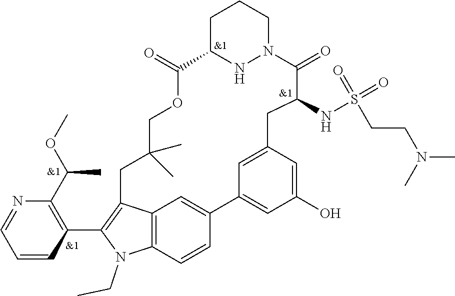 |
| A191 | 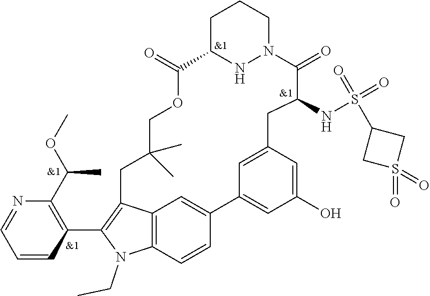 |
| A192 | 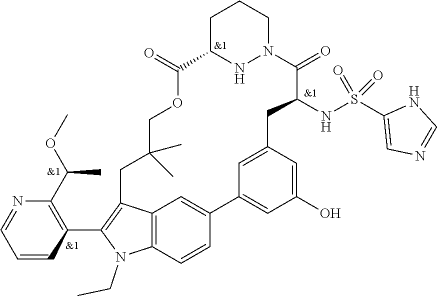 |
| A193 | 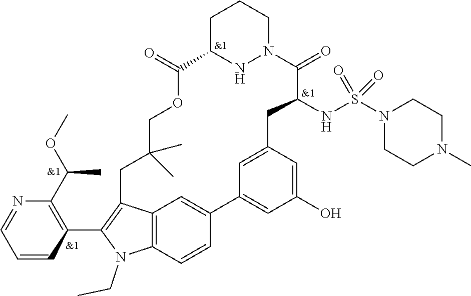 |
| A194 | 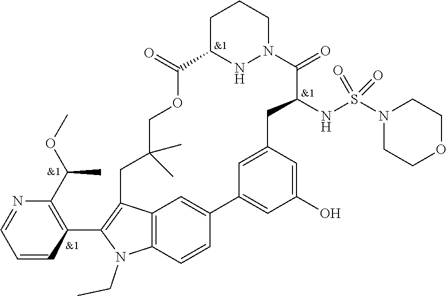 |
| A195 | 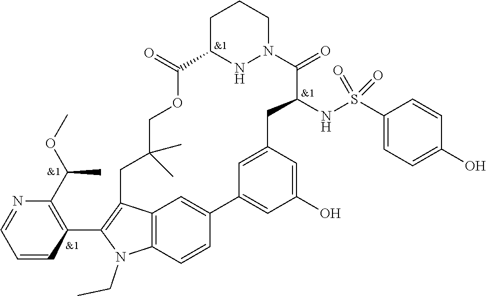 |
| A196 | 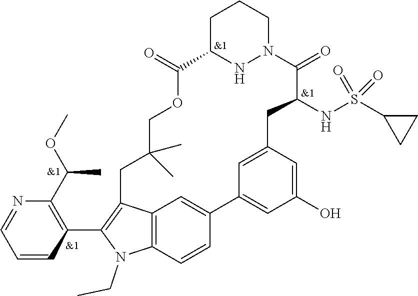 |
| A197 | 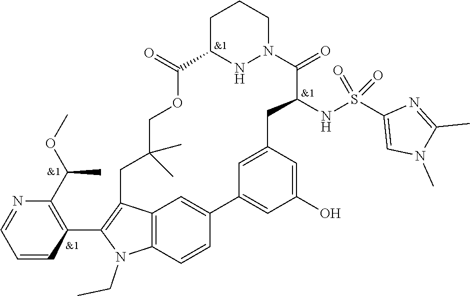 |
| A198 | 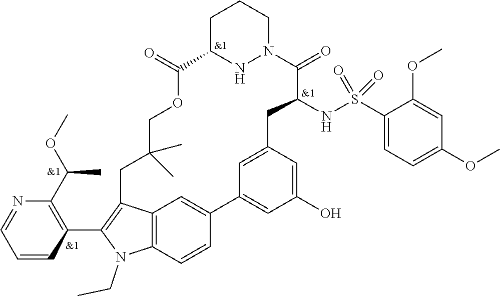 |
| A199 | 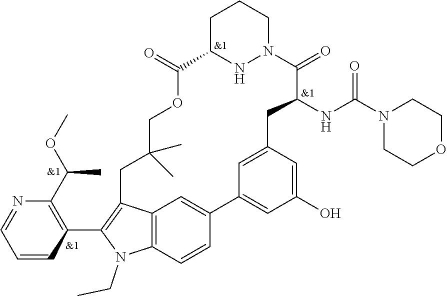 |
| A200 | 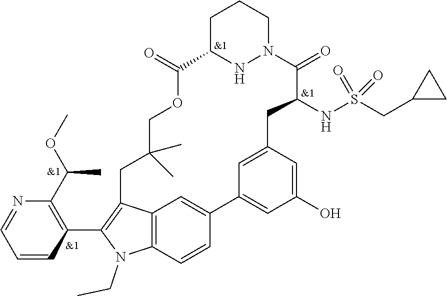 |
| A201 | 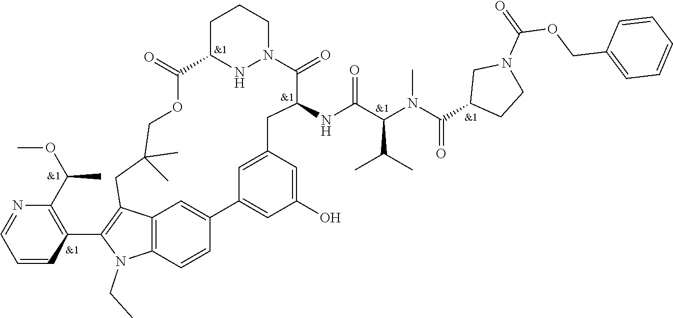 |
| A202 | 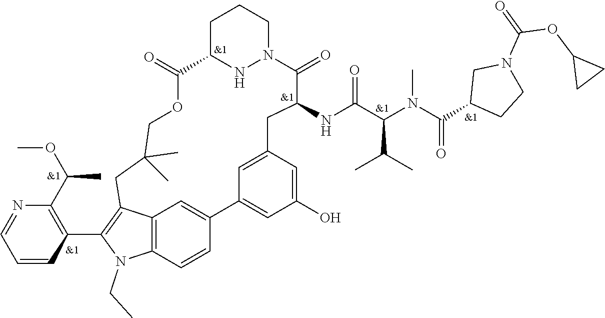 |
| A203 | 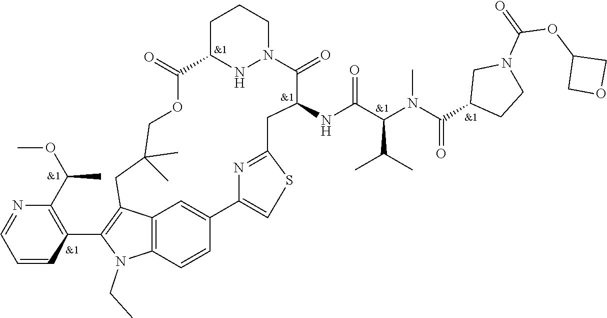 |
| A204 | 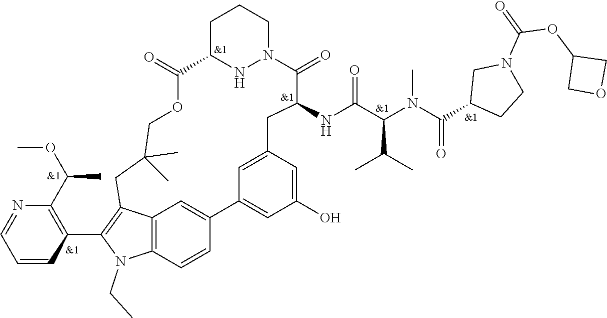 |
| A205 | 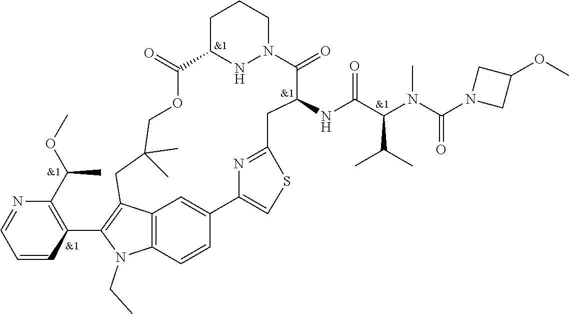 |
| A206 | 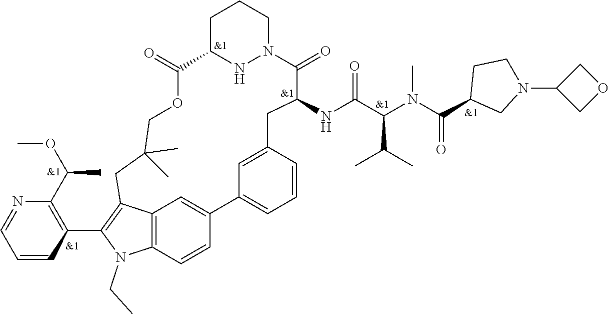 |
| A207 | 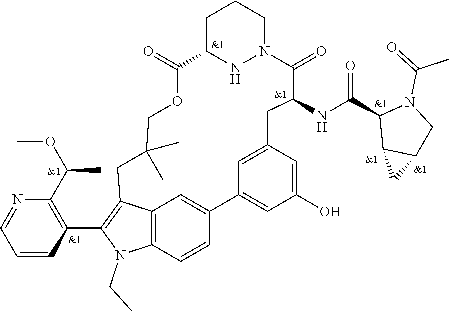 |
| A208 | 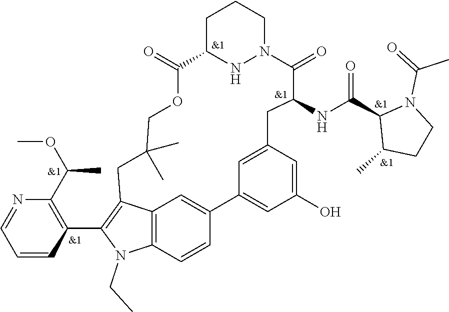 |
| A209 | 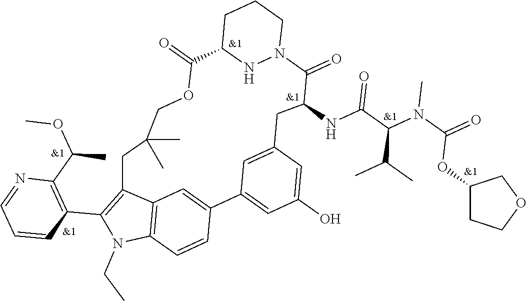 |
| A210 | 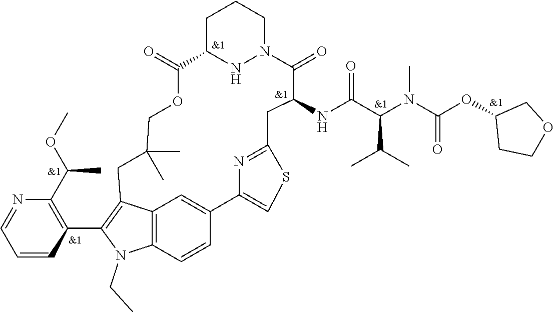 |
| A211 | 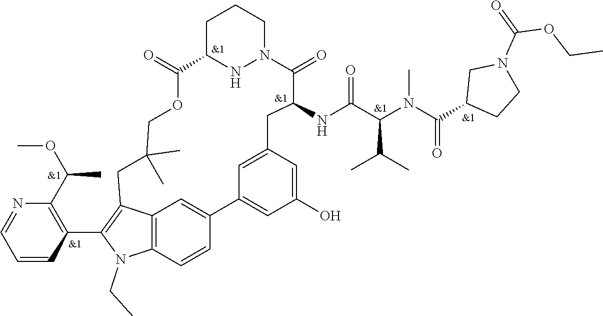 |
| A212 | 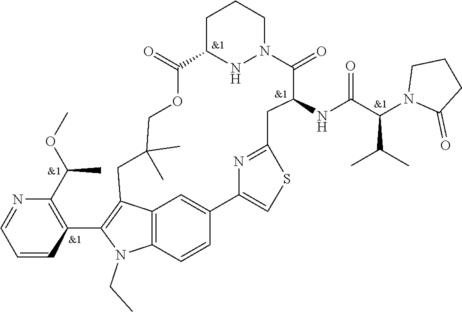 |
| A213 | 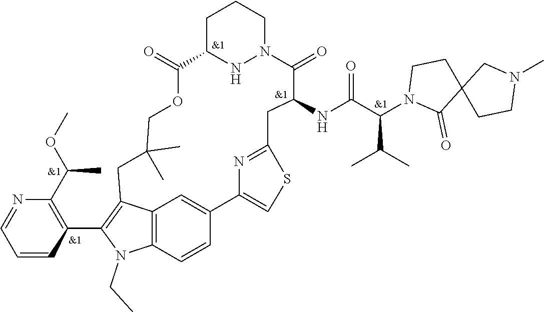 |
| A214 | 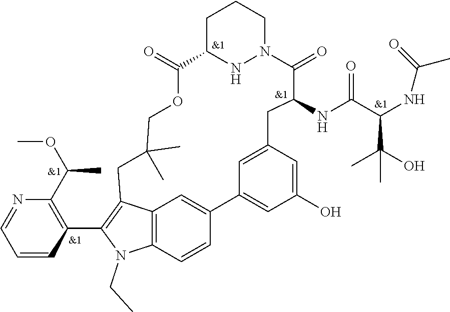 |
| A215 | 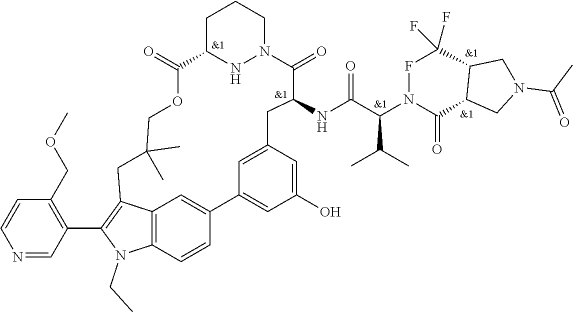 |
| A216 | 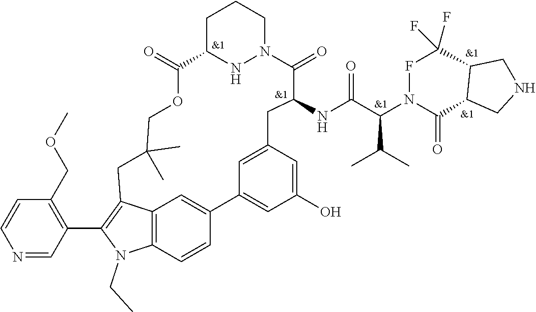 |
| A217 | 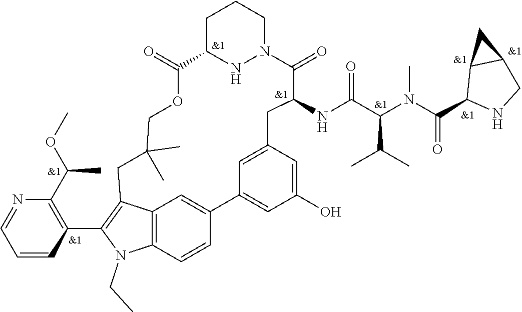 |
| A218 | 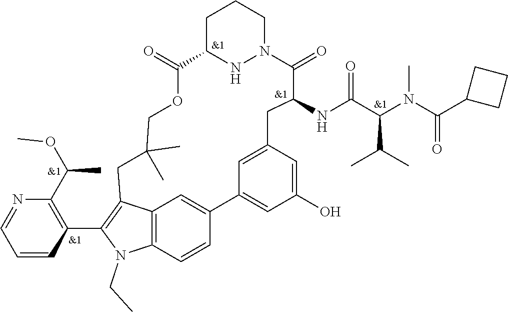 |
| A219 | 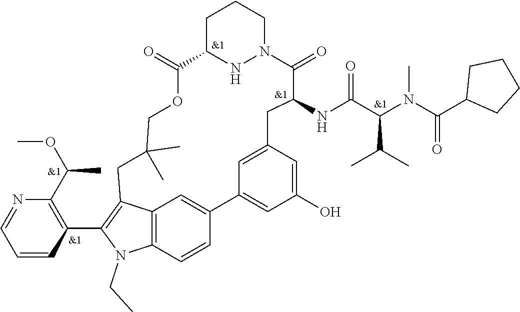 |
| A220 | 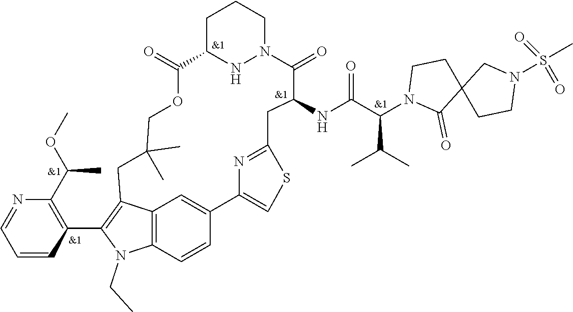 |
| A221 | 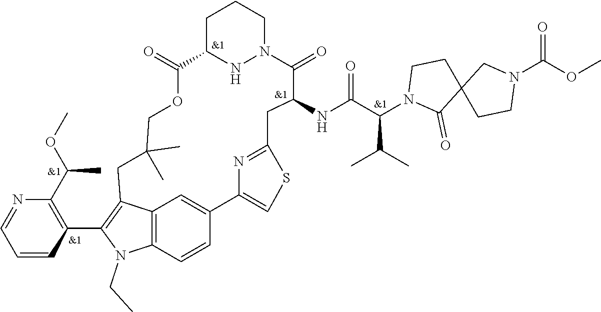 |
| A222 | 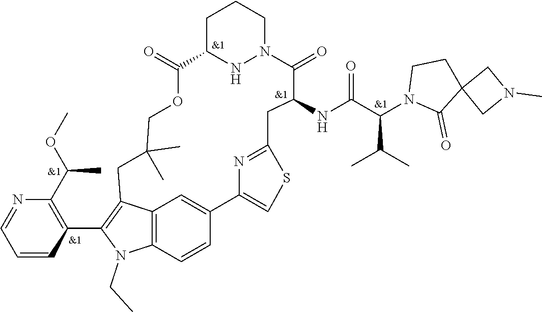 |
| A223 | 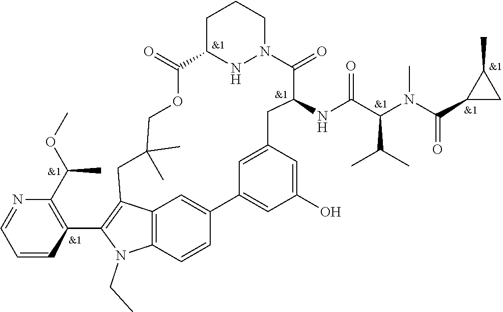 |
| A224 | 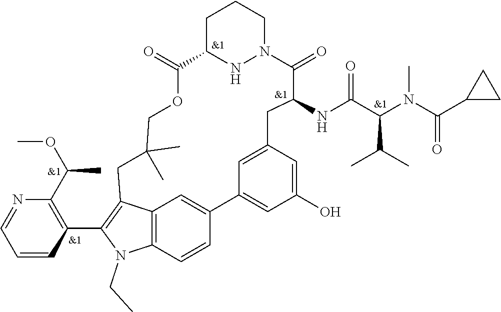 |
| A225 | 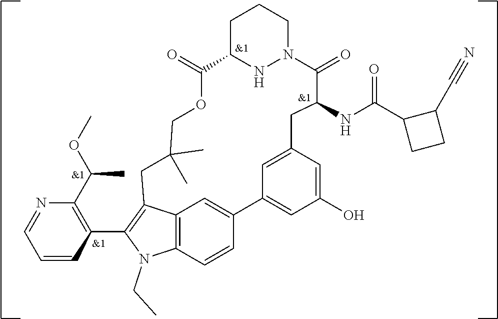 |
| A226 | 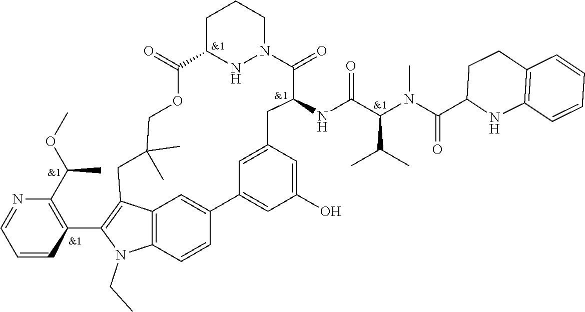 |
| A227 | 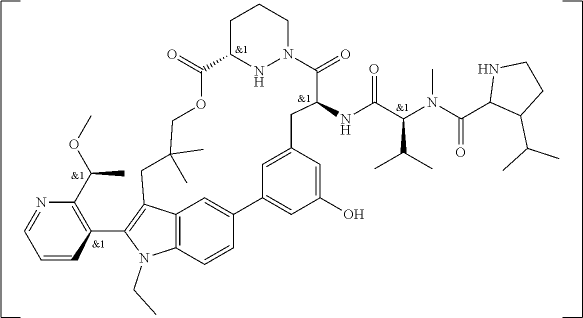 |
| A228 | 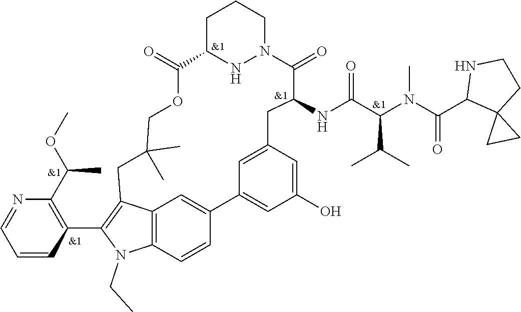 |
| A229 | 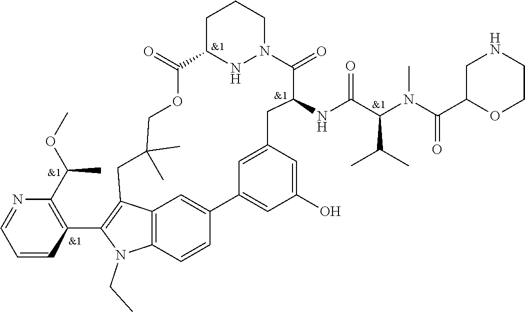 |
| A230 | 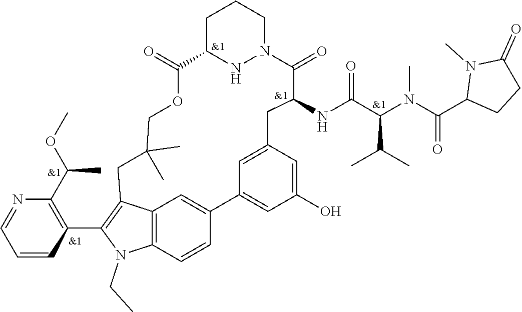 |
| A231 | 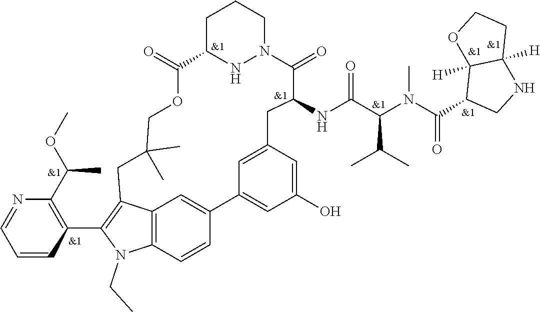 |
| A232 | 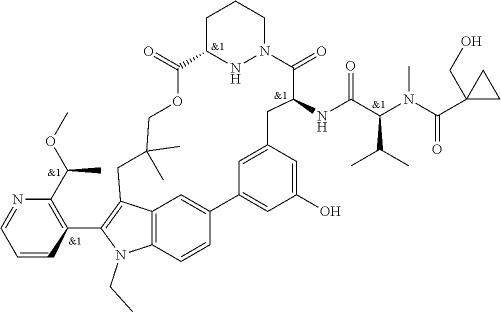 |
| A233 | 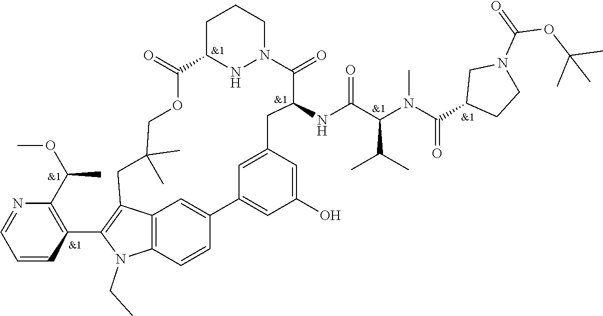 |
| A234 | 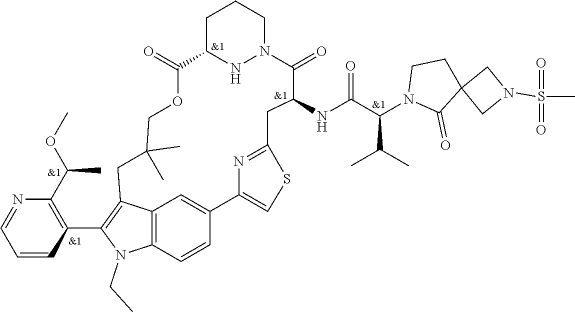 |
| A235 | 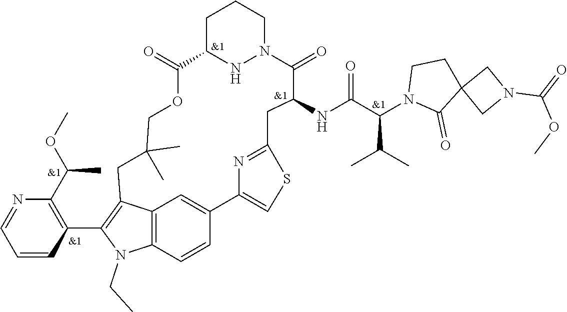 |
| A236 | 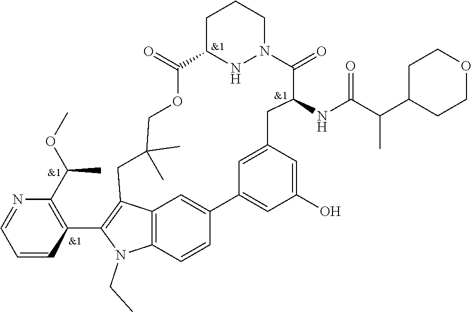 |
| A237 | 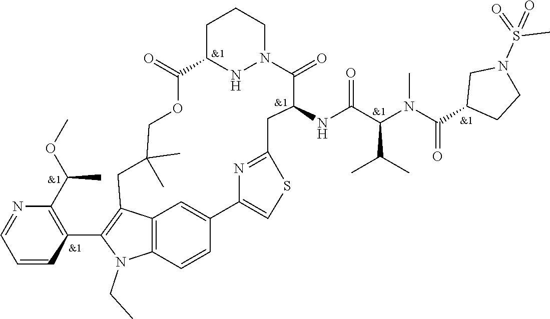 |
| A238 | 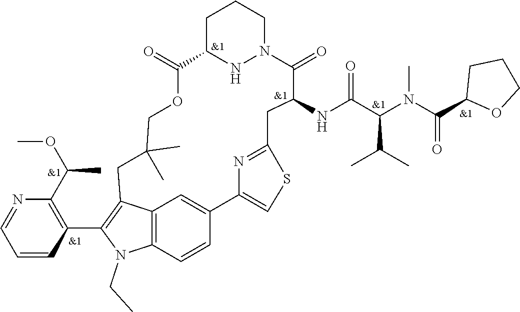 |
| A239 | 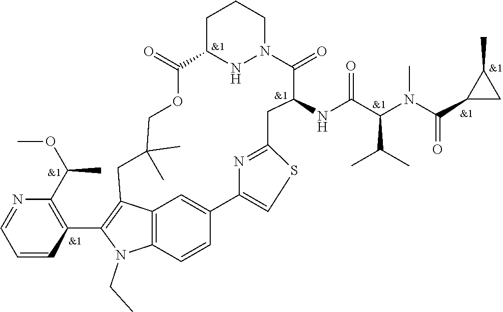 |
| A240 | 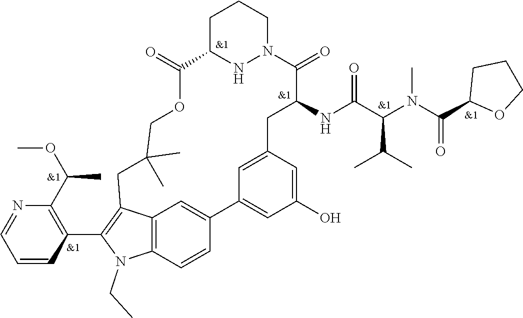 |
| A241 | 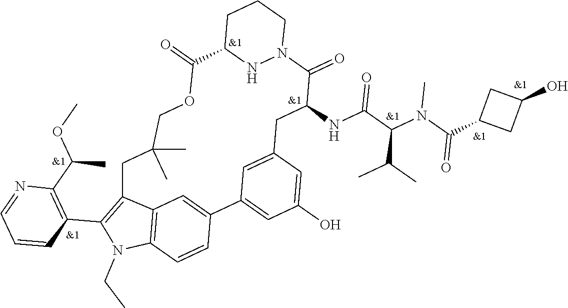 |
| A242 | 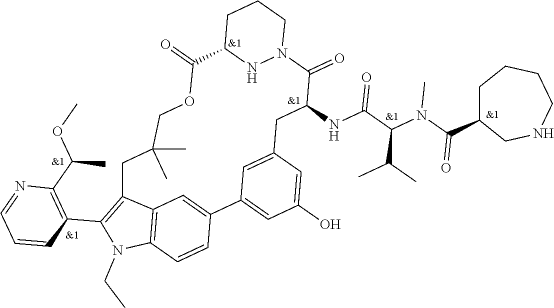 |
| A243 | 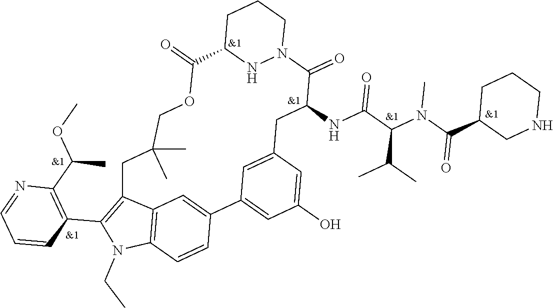 |
| A244 | 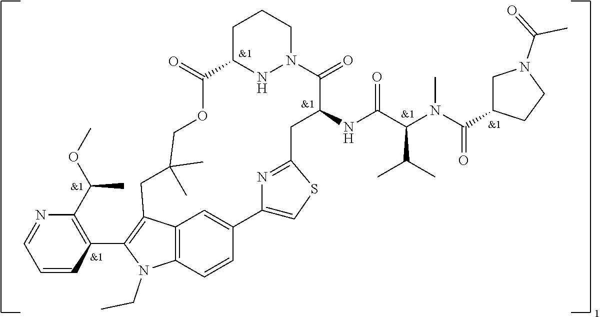 |
| A245 | 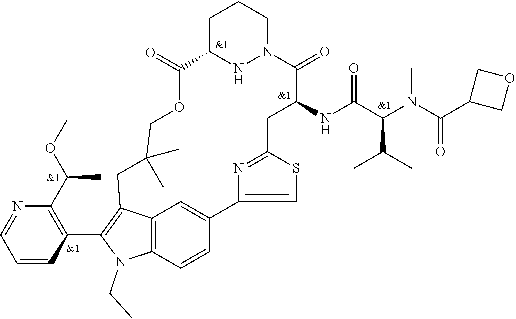 |
| A246 | 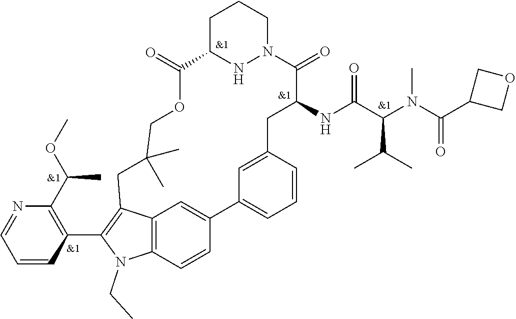 |
| A247 | 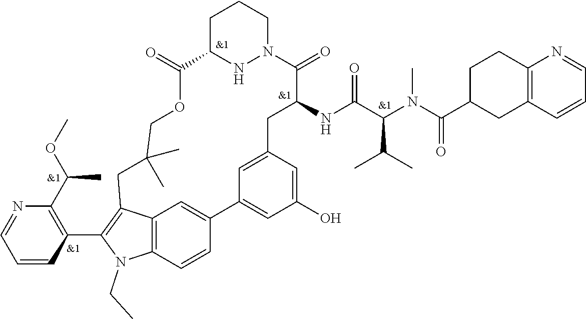 |
| A248 | 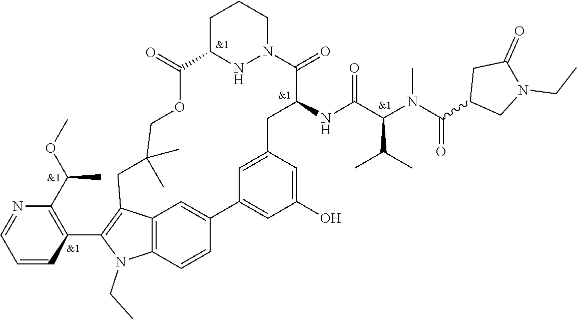 |
| A249 | 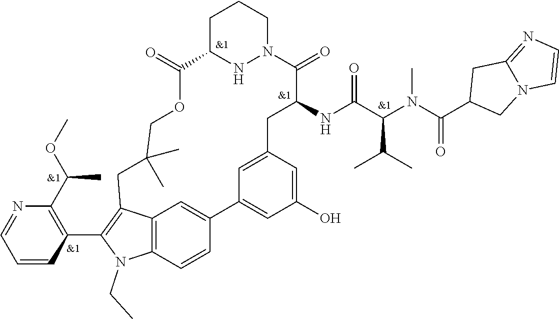 |
| A250 | 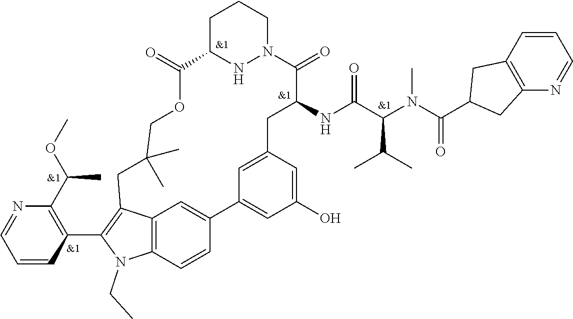 |
| A251 | 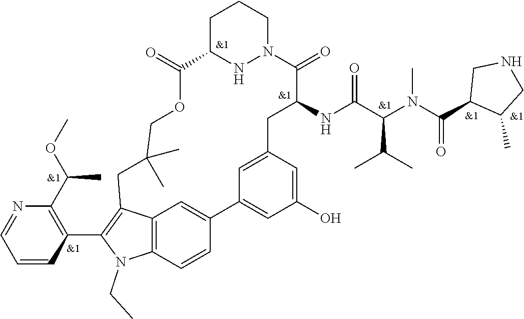 |
| A252 | 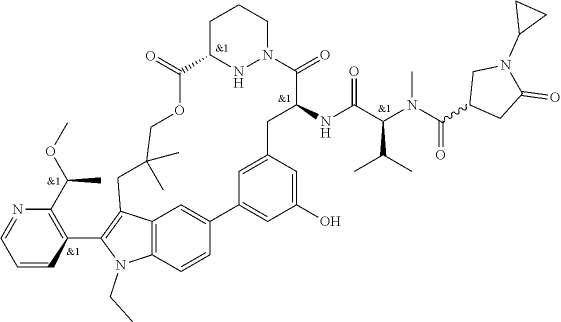 |
| A253 | 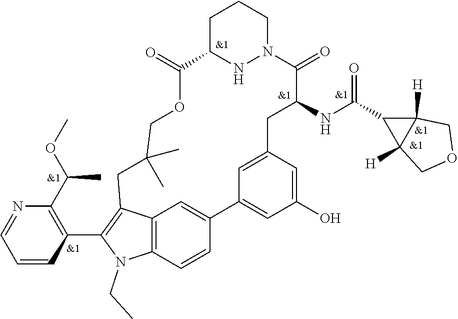 |
| A254 | 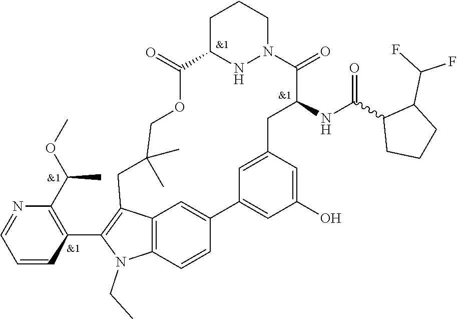 |
| A255 | 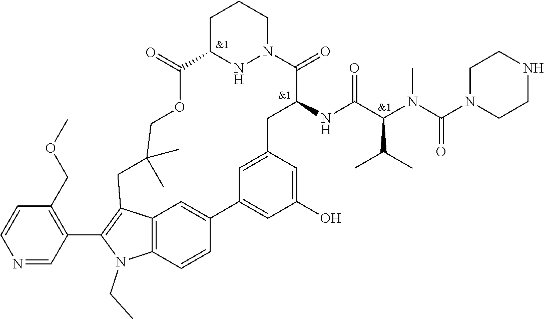 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| A256 | 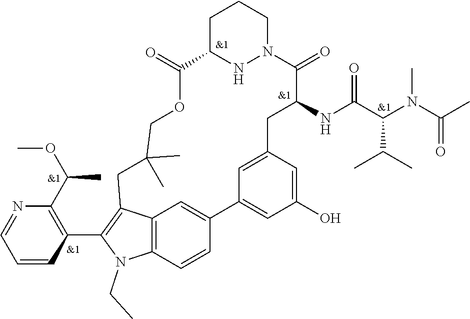 |
| A257 | 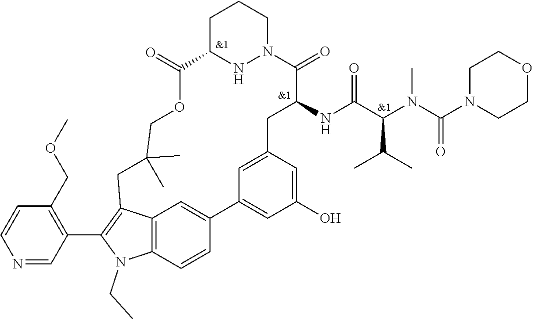 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| A258 | 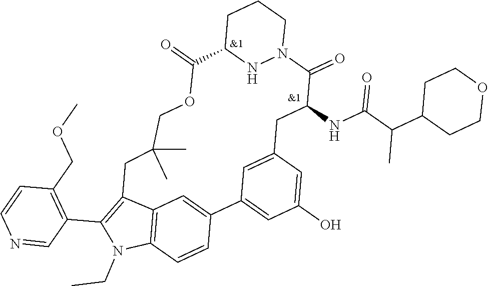 [SMILES: CCn1c(-c2cnccc2COC)c2c3cc(ccc31)-c1cc(O)cc(c1)C[C@H](NC(=O)C(C)C1CCOCC1)C(=O)N1CCC[C@H](N1)C(=O)OCC(C)(C)C2] |
| A259 | 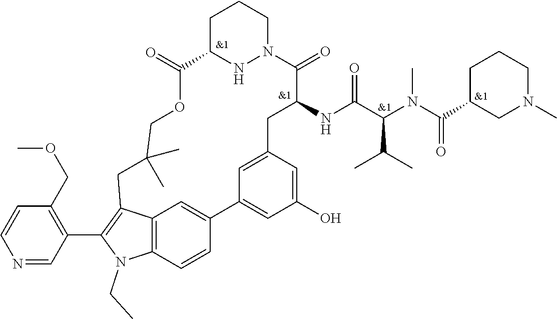 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| A260 | 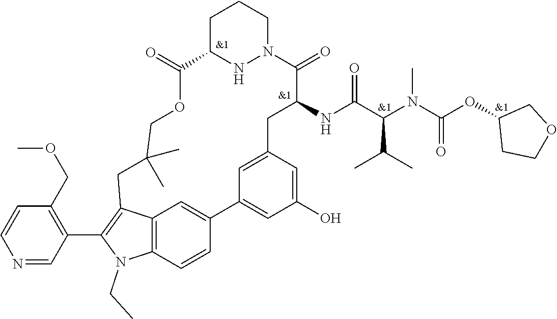 [SMILES: CCn1c(-c2cnccc2COC)c2c3cc(ccc31)-c1cc(O)cc(c1)C[C@H](NC(=O)[C@H](C(C)C)N(C)C(=O)O[C@H]1CCOC1)C(=O)N1CCC[C@H](N1)C(=O)OCC(C)(C)C2] |
| A261 | 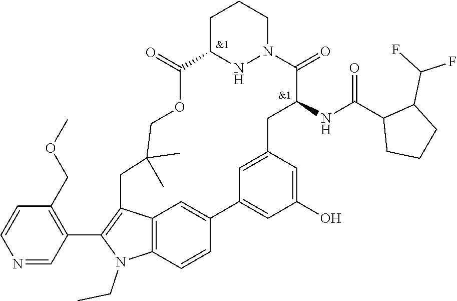 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| A262 | 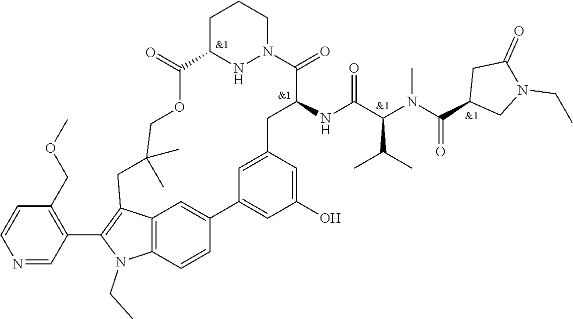 [SMILES: CCN1C[C@@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)CC1=O] |
| A263 | 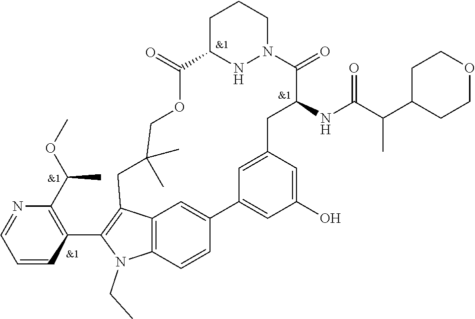 |
| A264 | 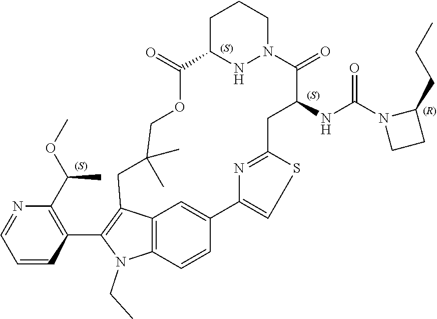 |
| A265 | 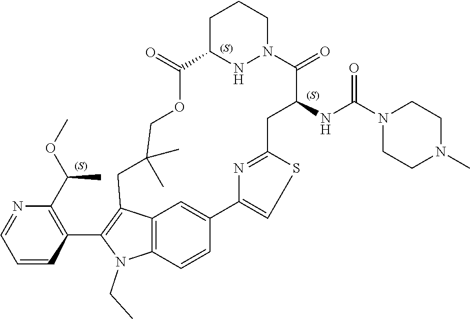 |
| A266 | 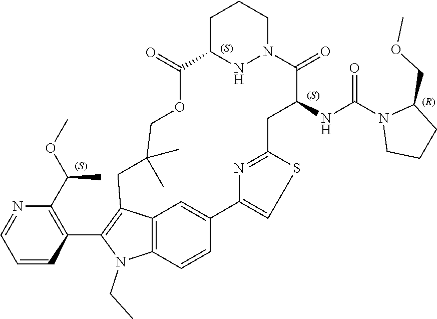 |
| A267 | 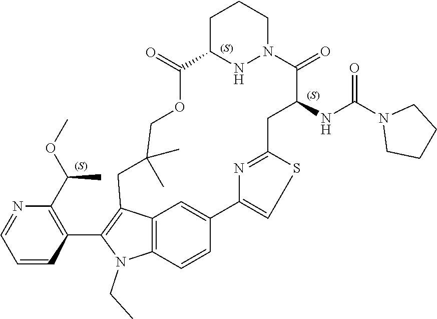 |
| A268 | 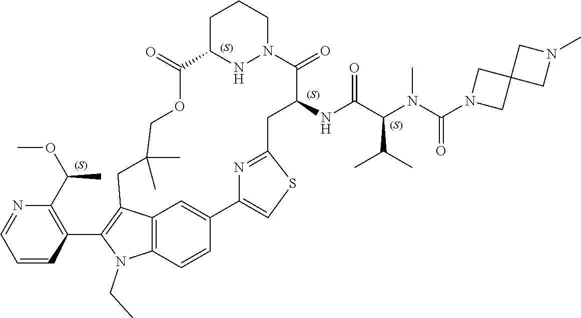 |
| A270 | 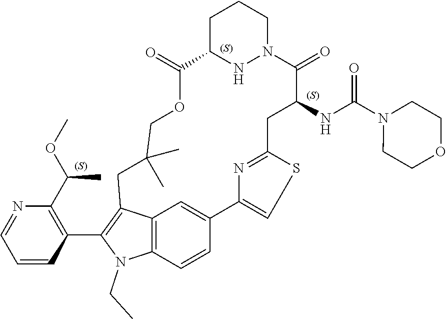 |
| A271 | 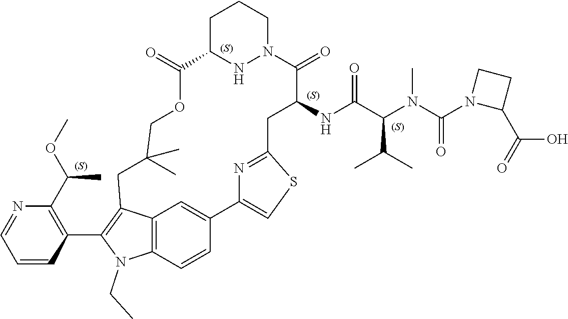 |
| A272 | 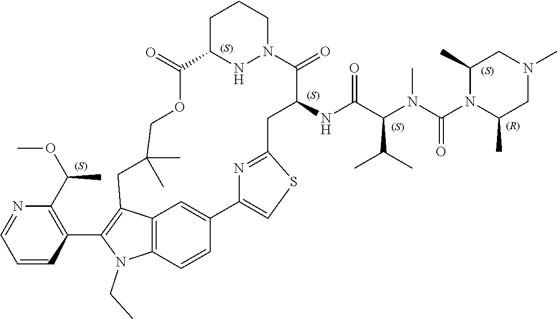 |
| A273 | 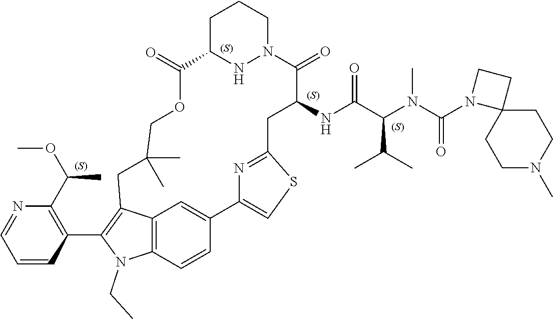 |
| A274 | 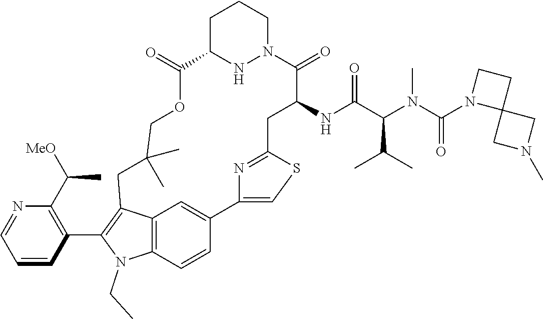 |
| A275 | 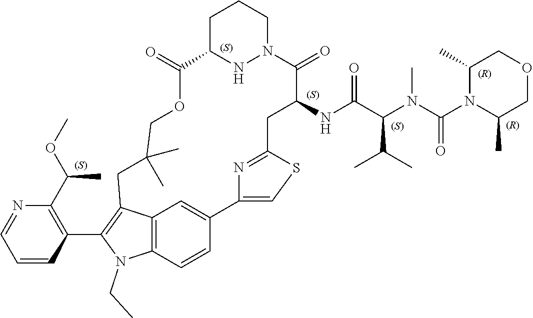 |
| A276 | 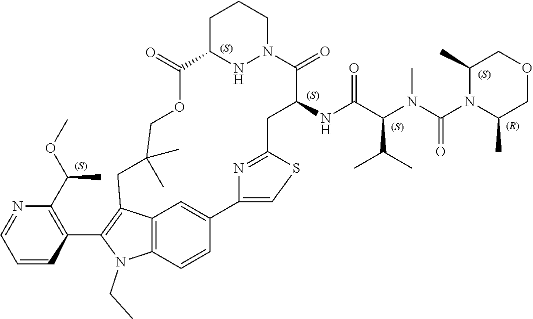 |
| A277 | 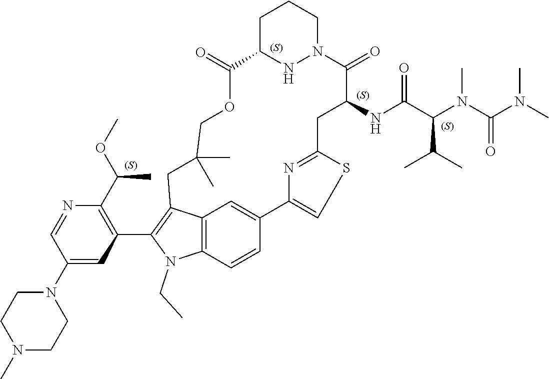 |
| A278 | 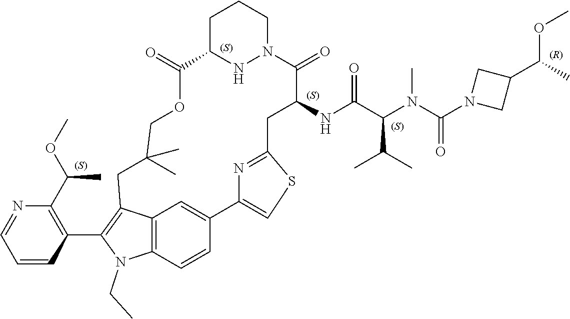 |
| A279 | 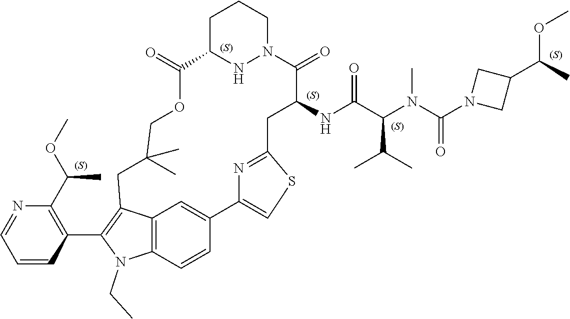 |
| A280 | 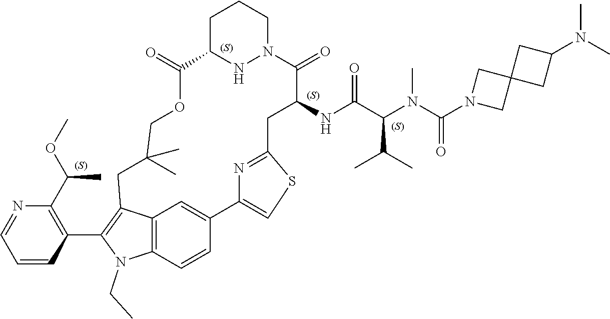 |
| A281 | 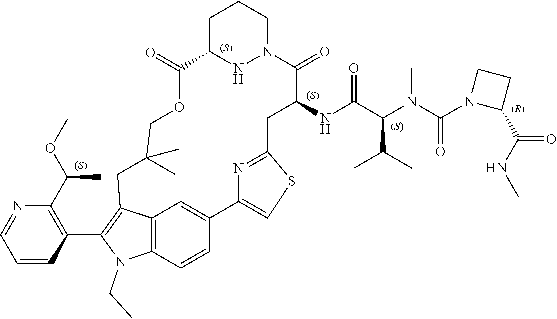 |
| A282 | 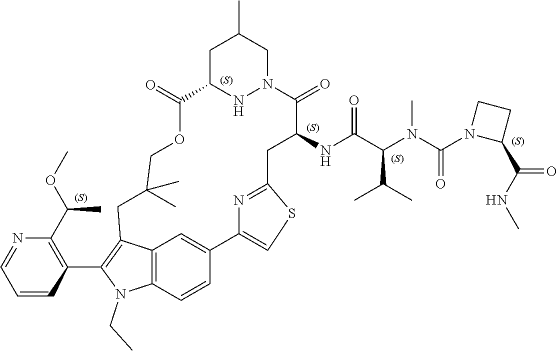 |
| A283 | 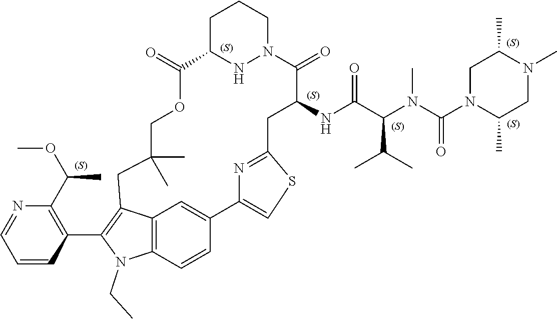 |
| A284 | 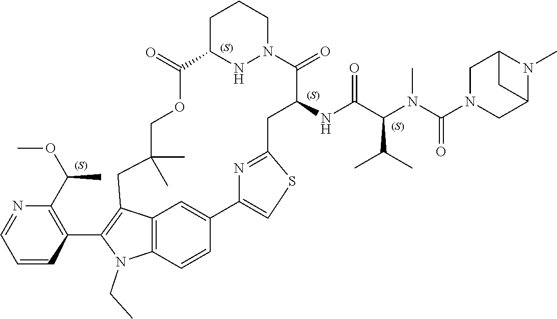 |
| A285 | 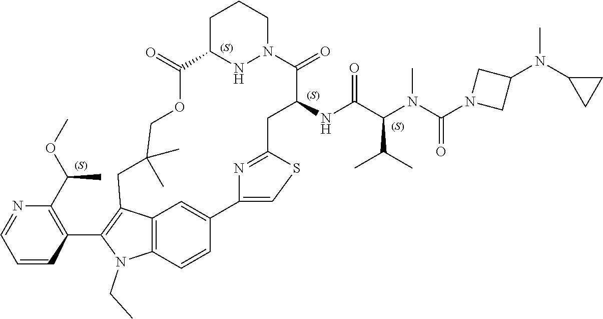 |
| A286 | 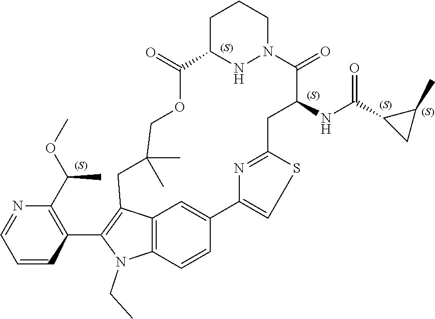 |
| A287 | 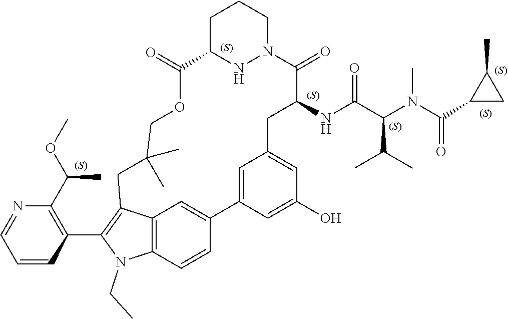 |
| A288 | 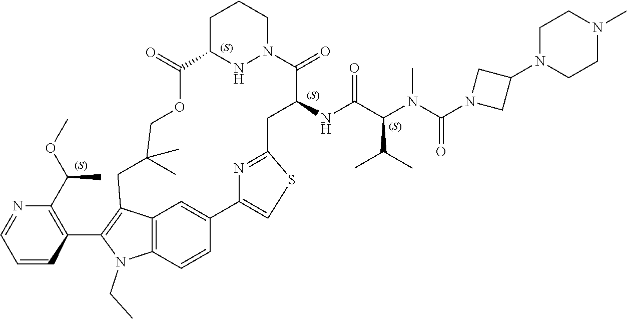 |
| A289 | 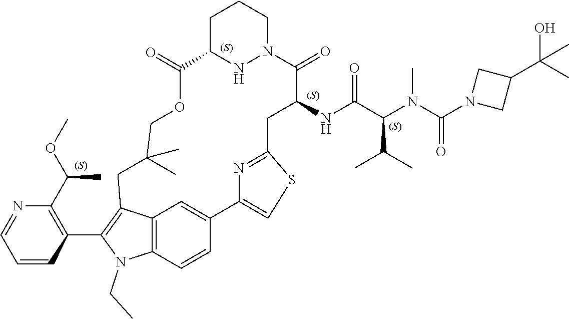 |
| A290 | 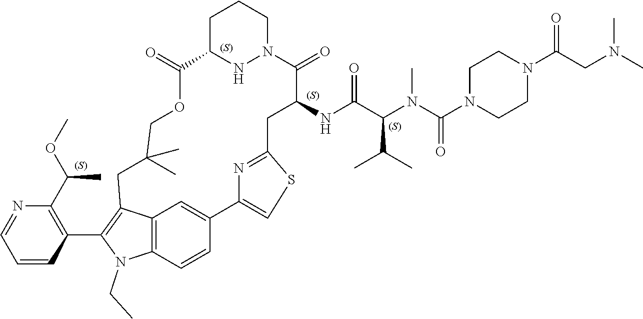 |
| A291 | 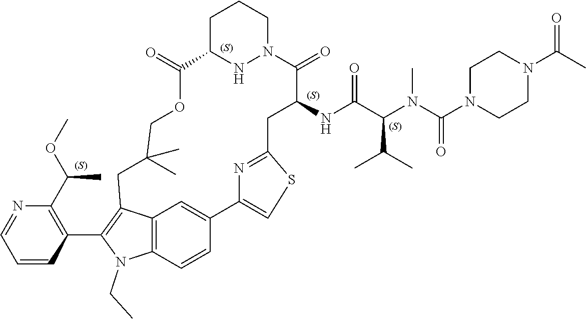 |
| A292 | 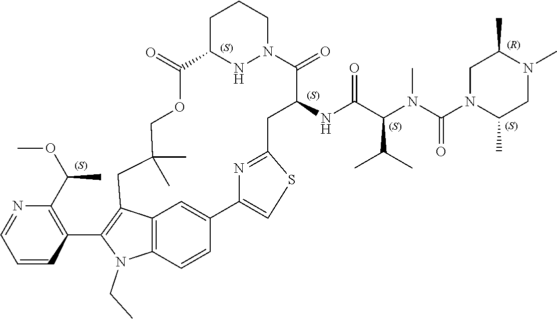 |
| A293 | 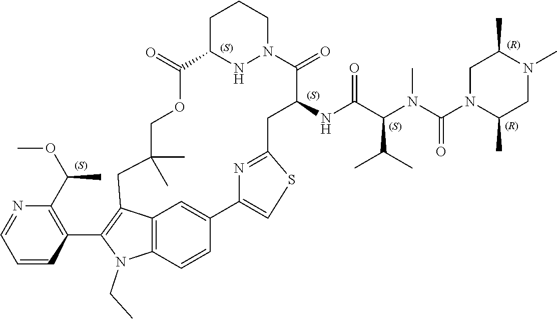 |
| A294 | 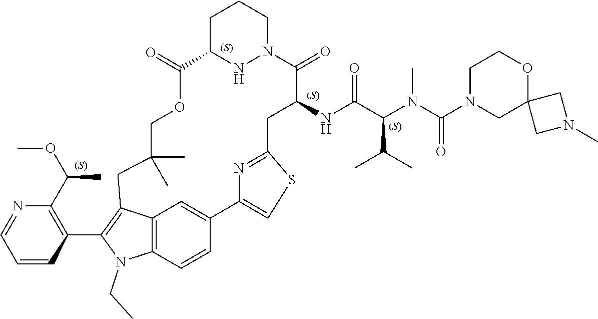 |
| A295 | 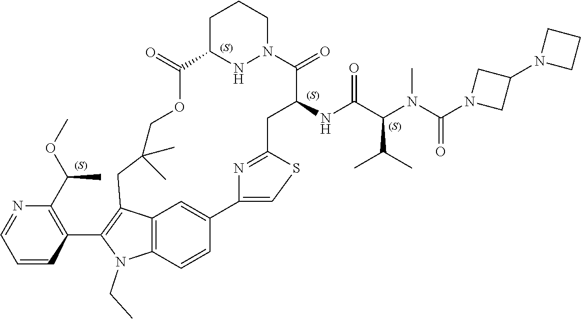 |
| A296 | 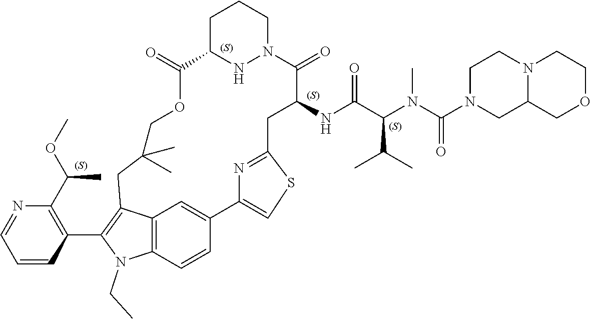 |
| A297 | 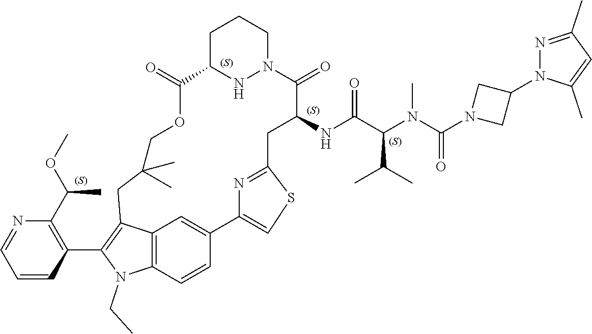 |
| A298 | 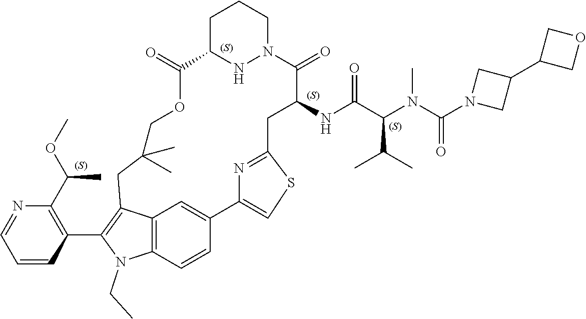 |
| A299 | 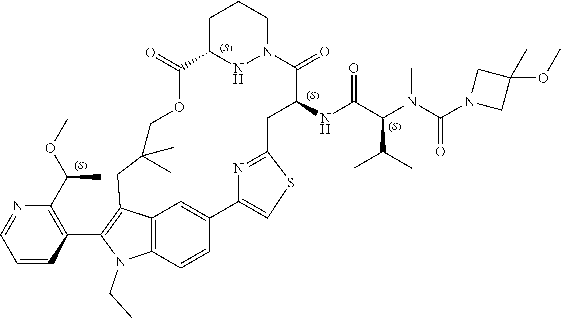 |
| A300 | 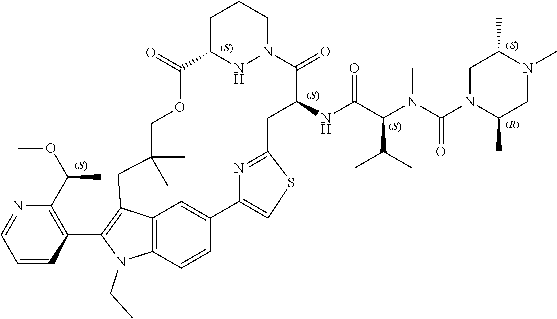 |
| A301 | 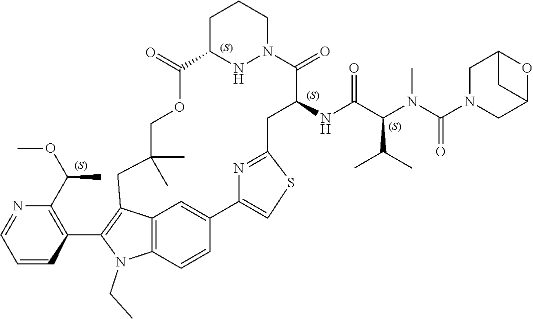 |
| A302 | 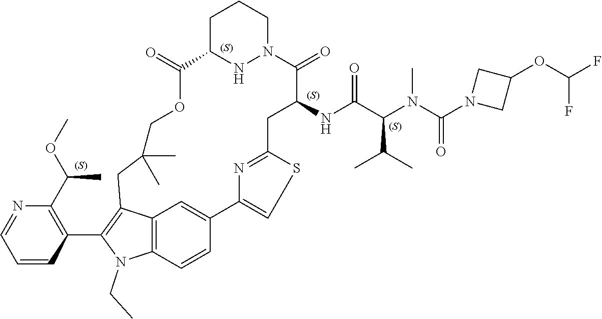 |
| A303 | 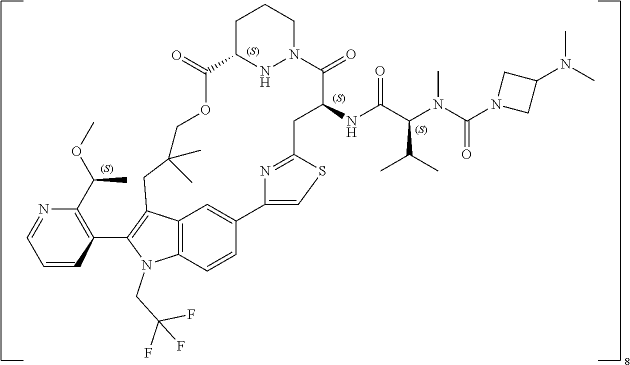 |
| A304 | 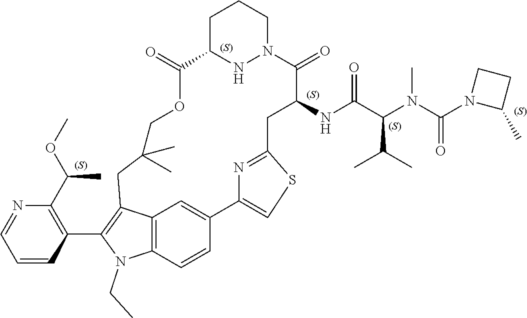 |
| A305 | 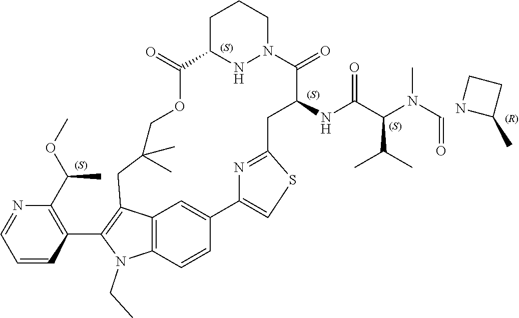 |
| A306 | 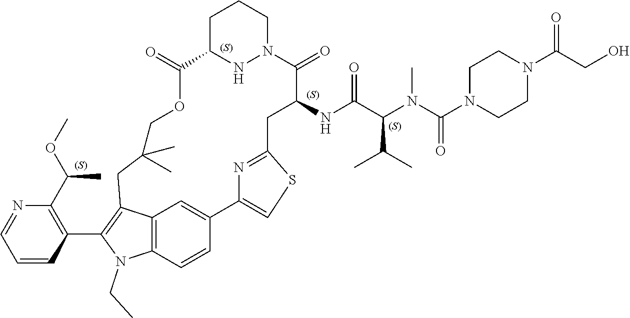 |
| A307 | 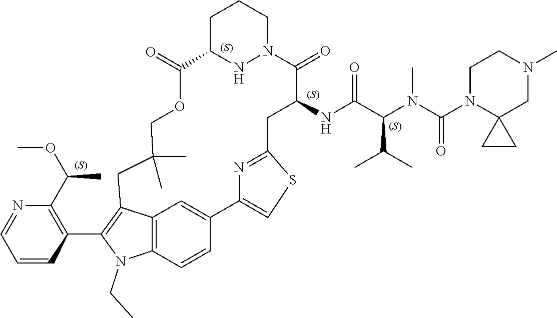 |
| A308 | 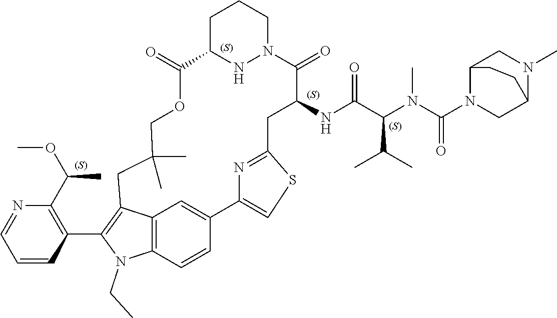 |
| A309 | 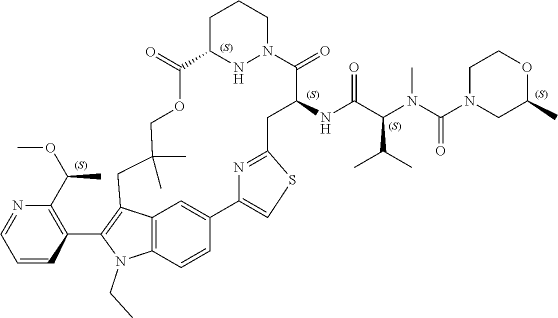 |
| A310 | 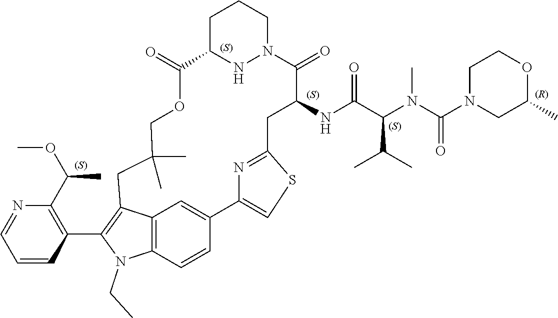 |
| A311 | 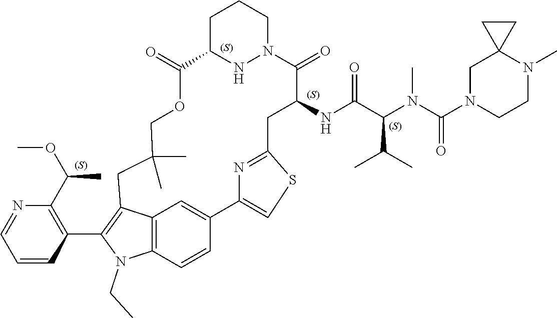 |
| A312 | 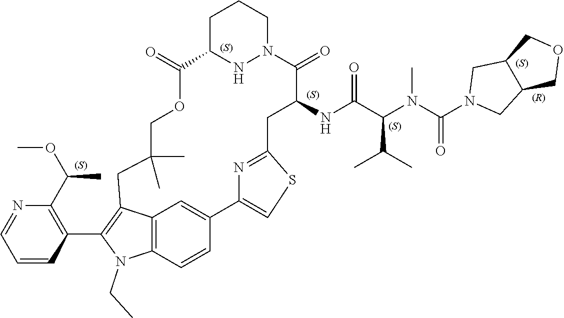 |
| A313 | 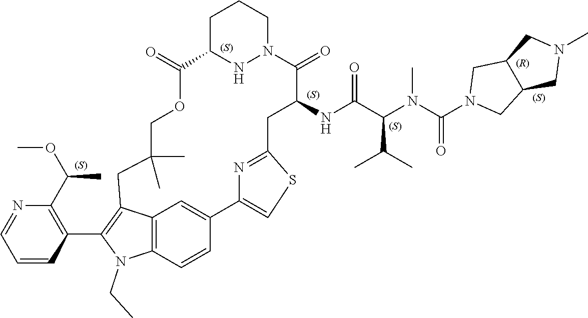 |
| A314 | 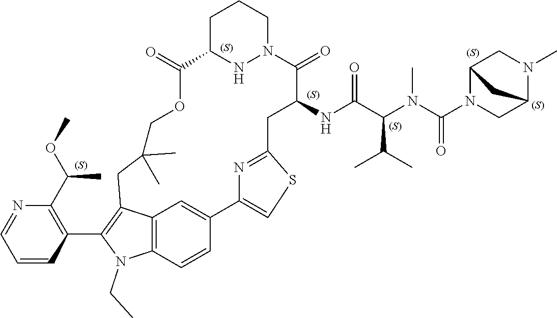 |
| A315 | 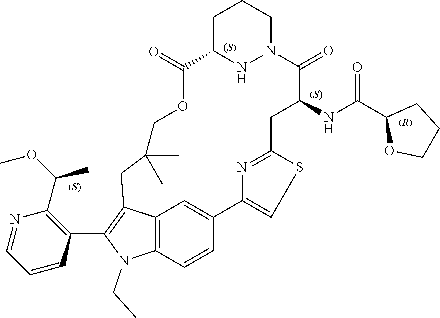 |
| A316 | 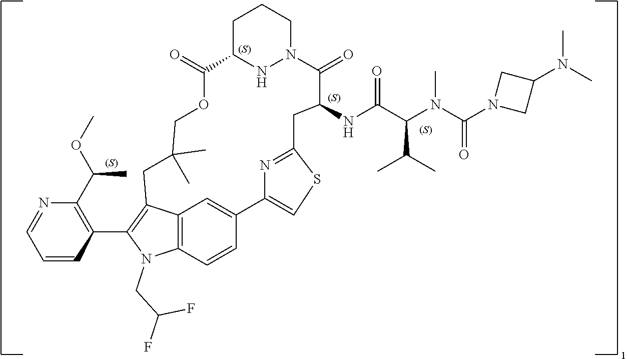 |
| A317 | 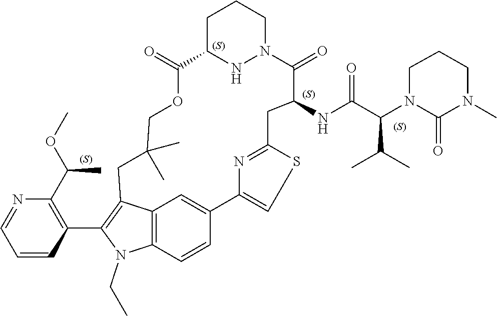 |
| A318 | 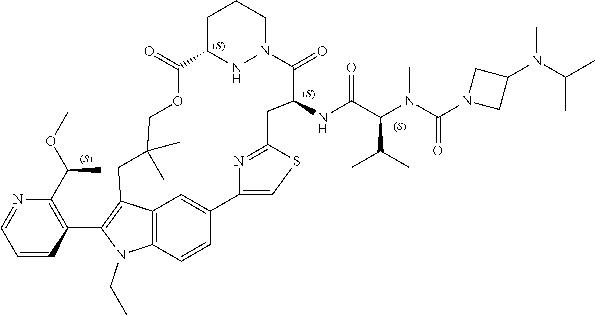 |
| A319 | 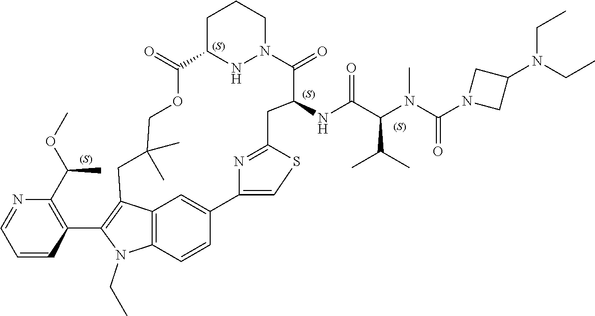 |
| A320 | 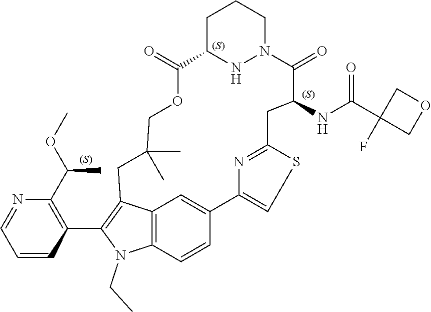 |
| A321 | 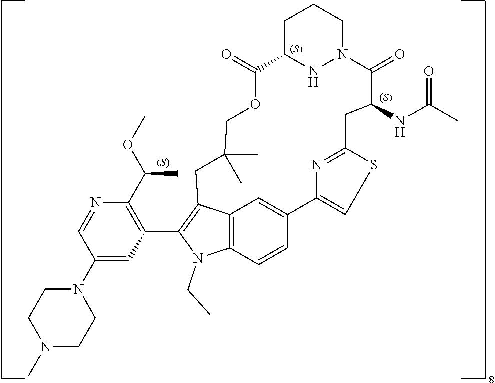 |
| A322 | 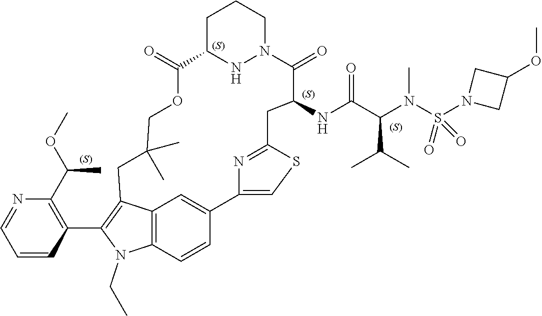 |
| A323 | 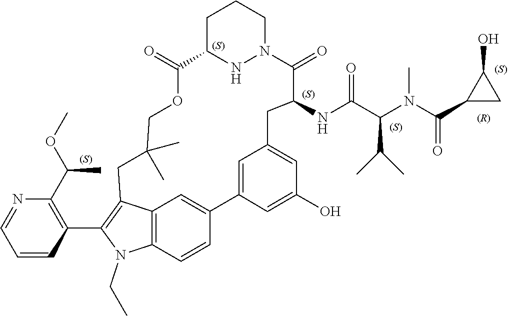 |
| A324 | 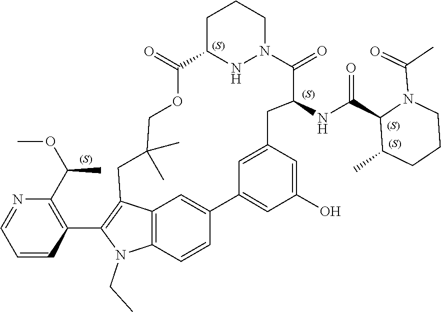 |
| A325 | 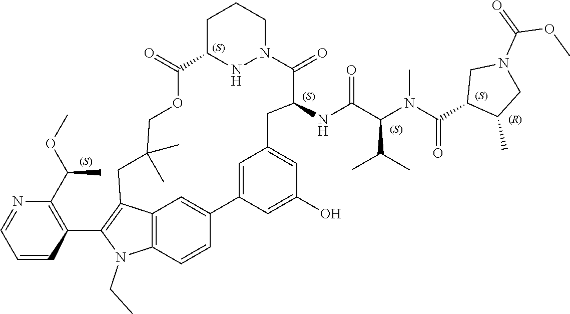 |
| A326 | 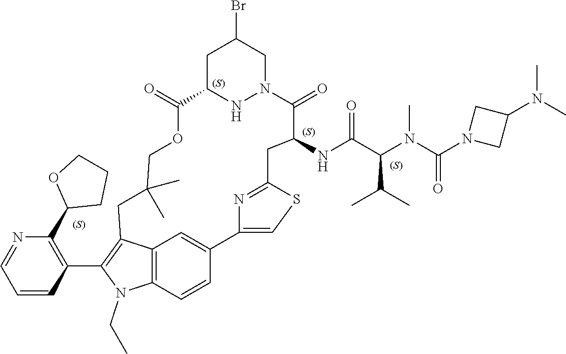 |
| A327 | 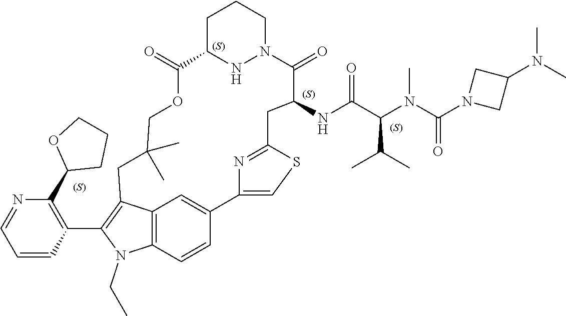 |
| A328 | 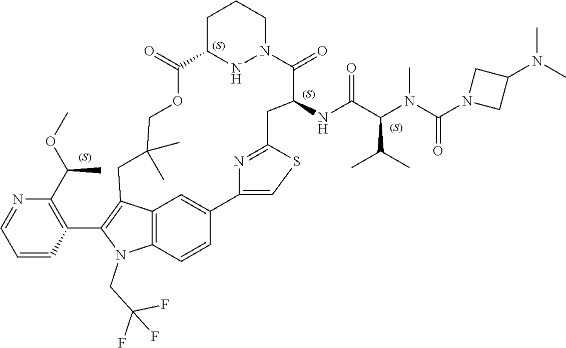 |
| A329 | 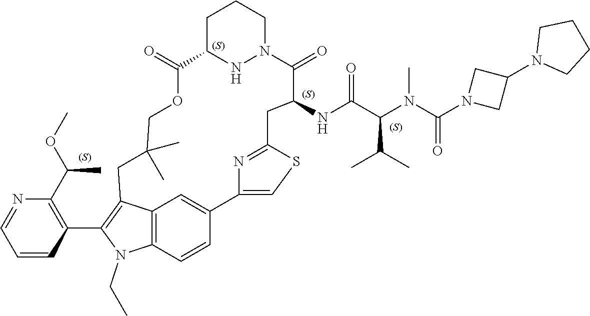 |
| A330 | 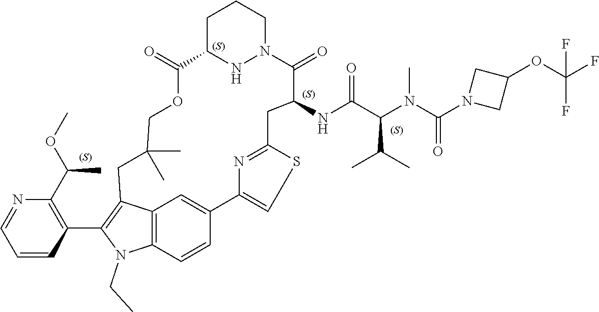 |
| A331 | 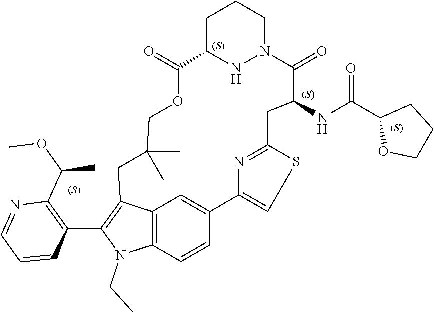 |
| A332 | 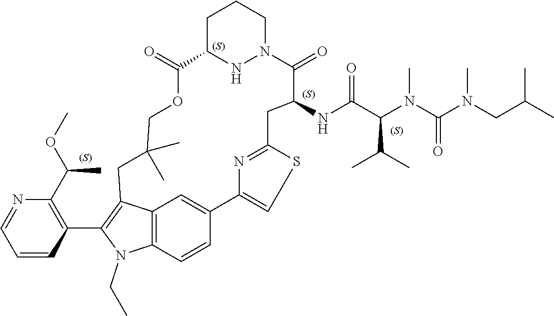 |
| A333 | 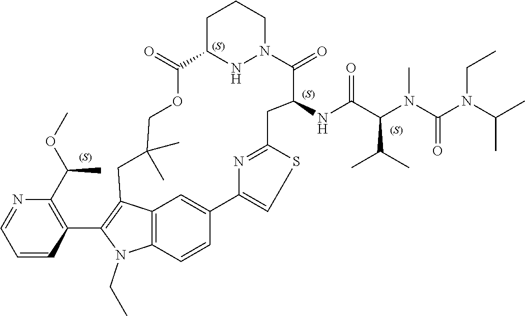 |
| A334 | 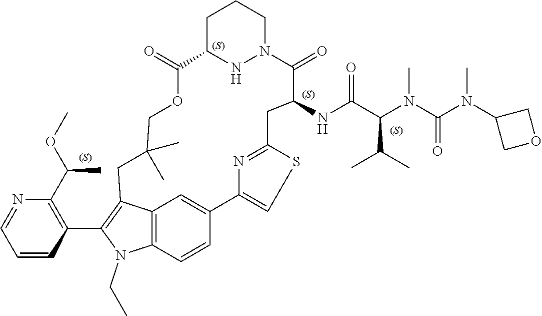 |
| A335 | 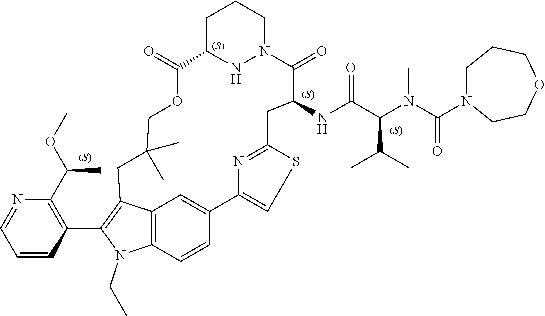 |
| A336 | 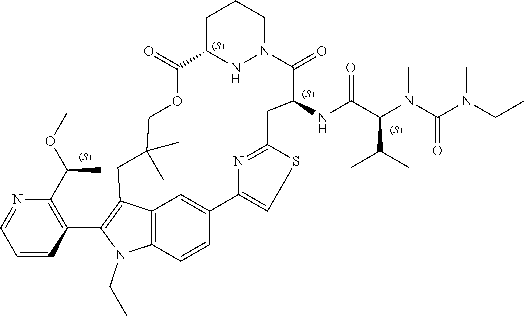 |
| A337 | 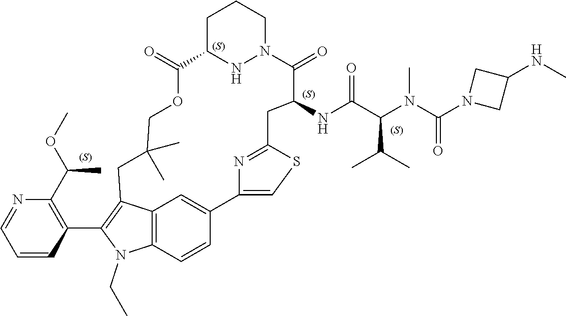 |
| A338 | 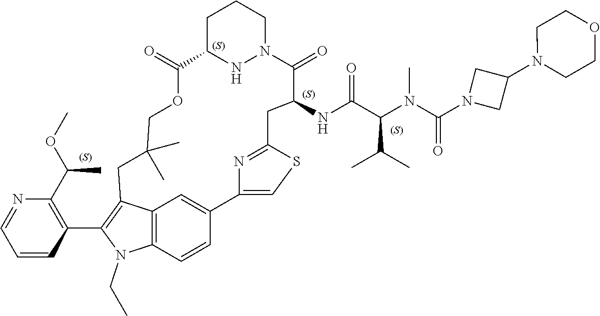 |
| A339 | 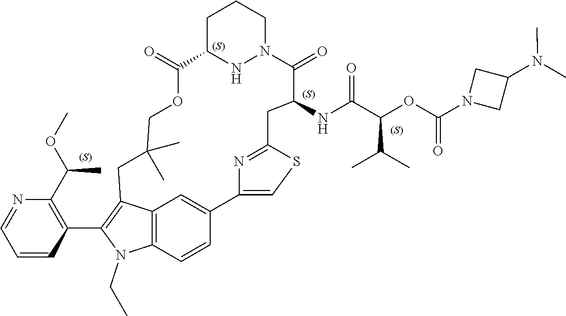 |
| A340 | 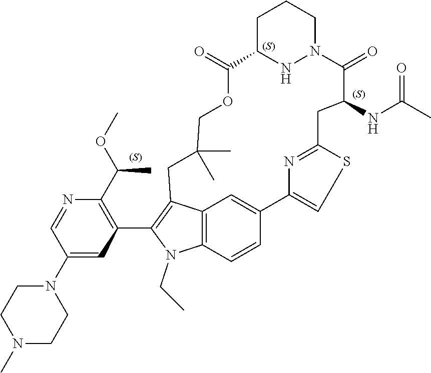 |
| A341 | 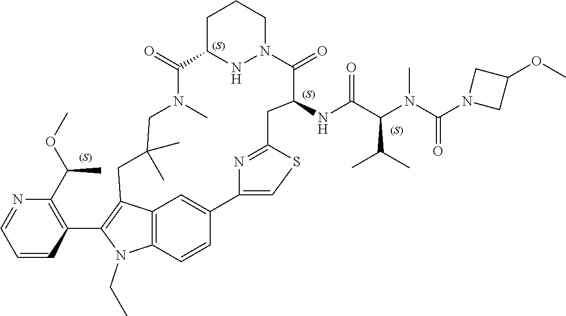 |
| A342 | 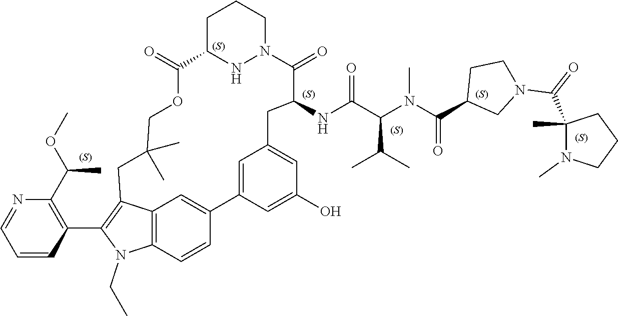 |
| A343 | 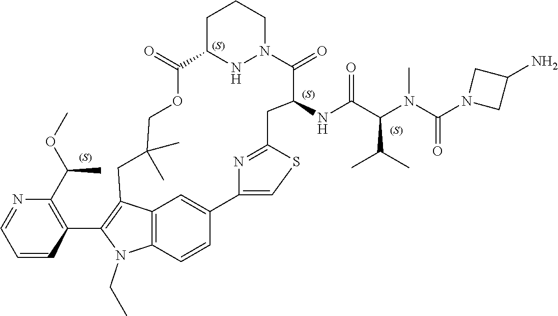 |
| A344 | 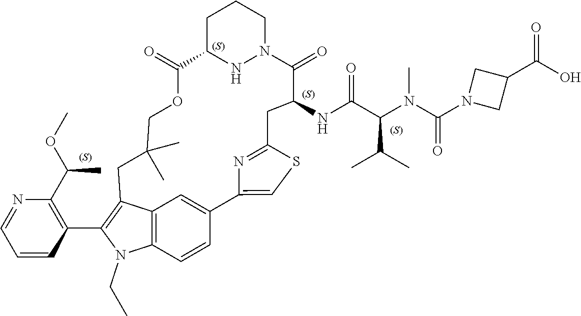 |
| A345 | 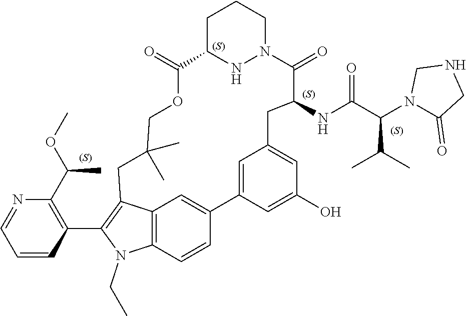 |
| A346 | 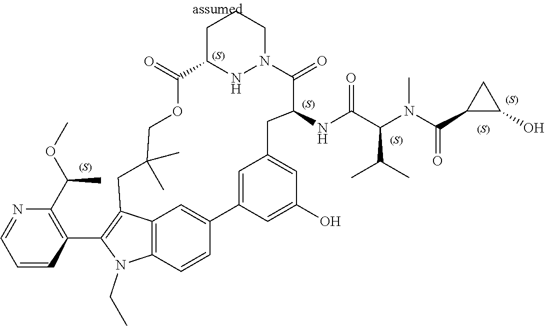 |
| A347 | 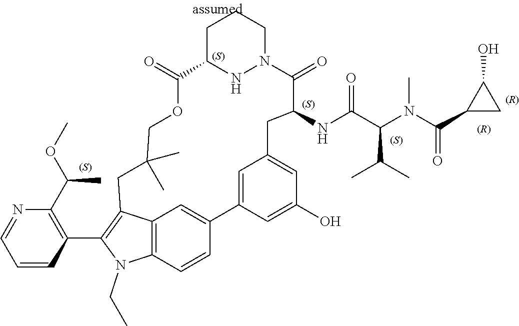 |
| A348 | 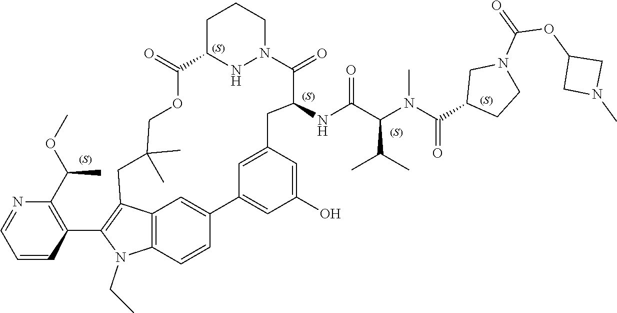 |
| A349 | 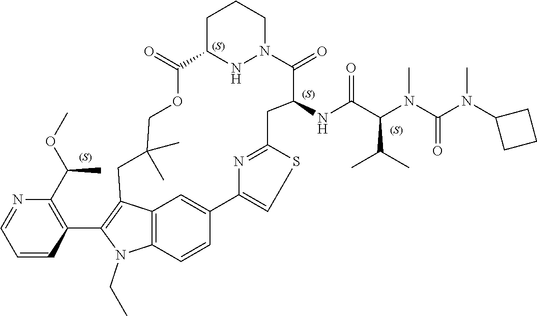 |
| A350 | 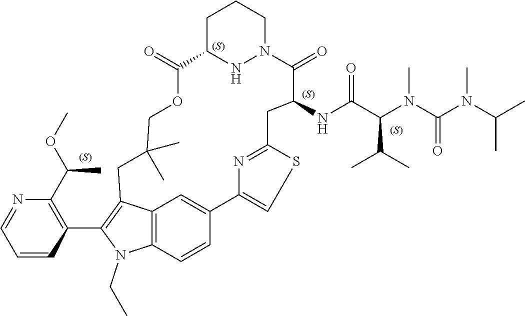 |
| A351 | 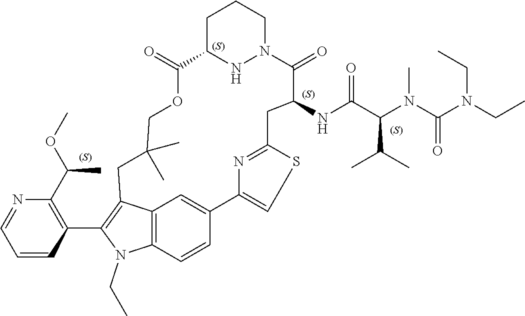 |
| A352 | 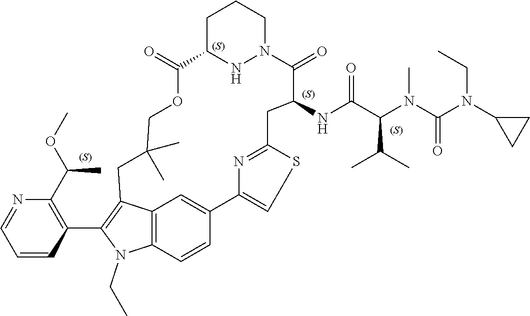 |
| A353 | 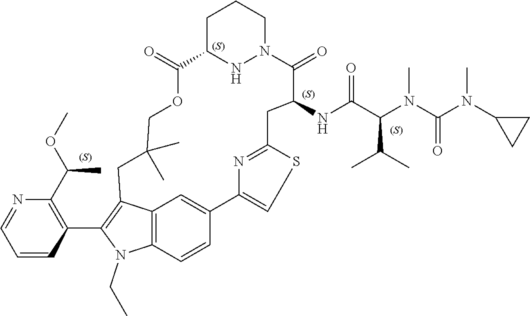 |
| A354 | 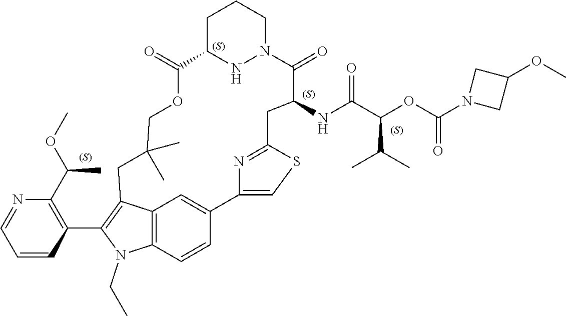 |
| A355 | 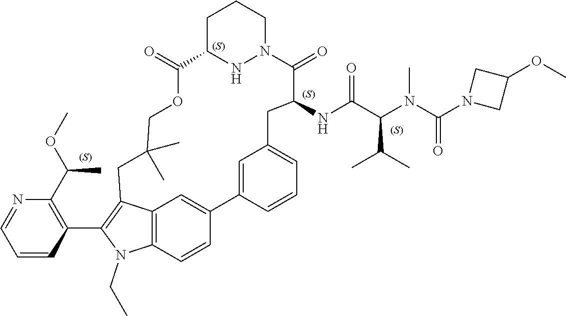 |
| A356 | 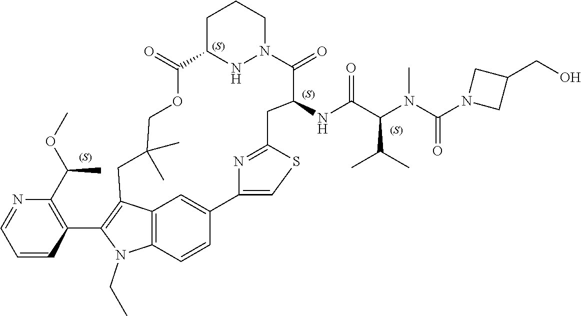 |
| A357 | 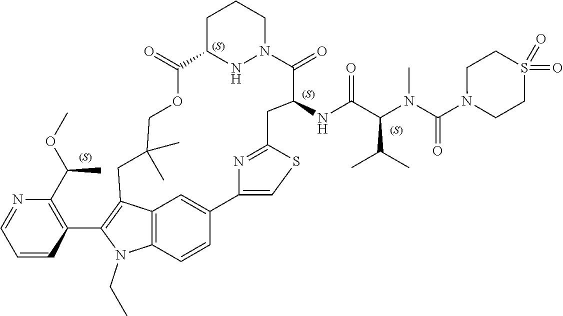 |
| A358 | 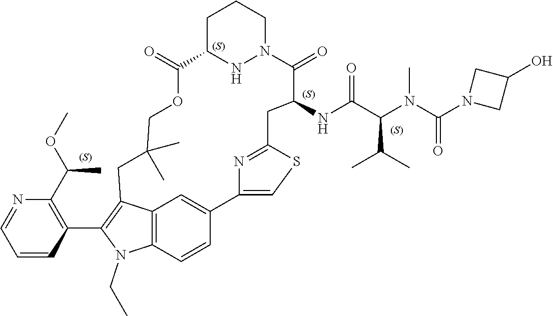 |
| A359 | 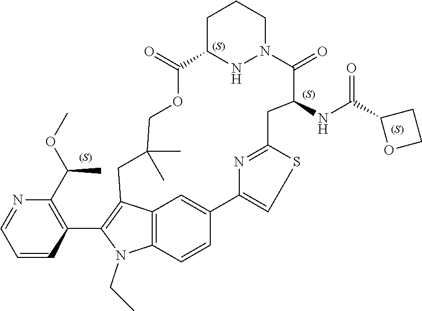 |
| A360 | 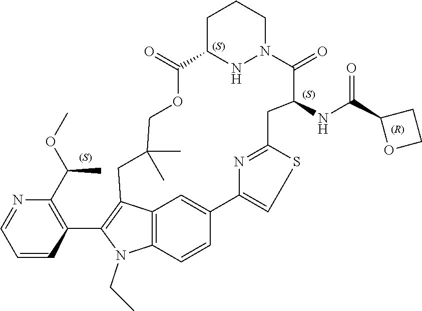 |
| A361 | 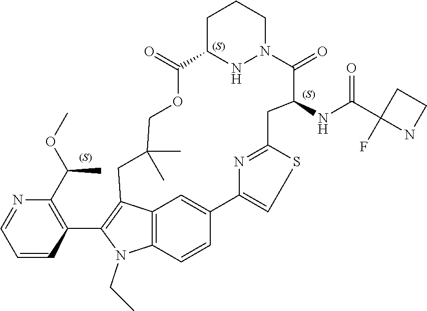 |
| A362 | 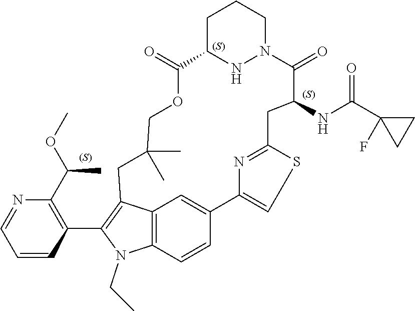 |
| A363 | 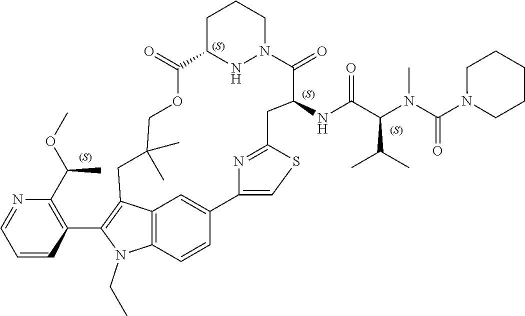 |
| A364 | 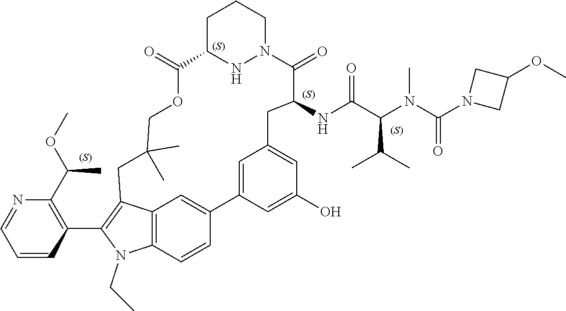 |
| A365 | 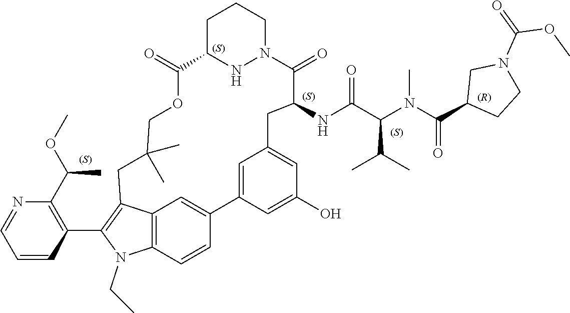 |
| A366 | 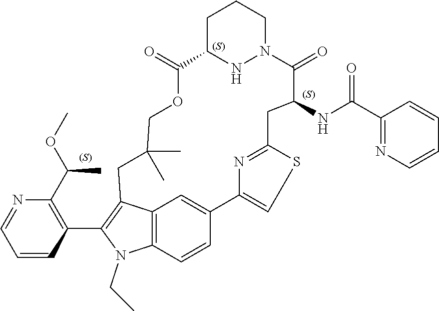 |
| A367 | 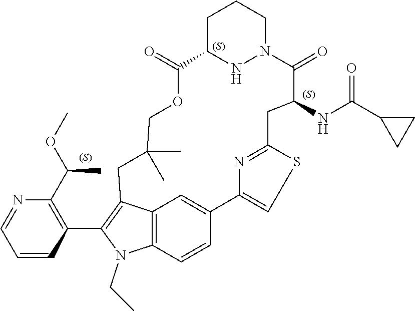 |
| A368 | 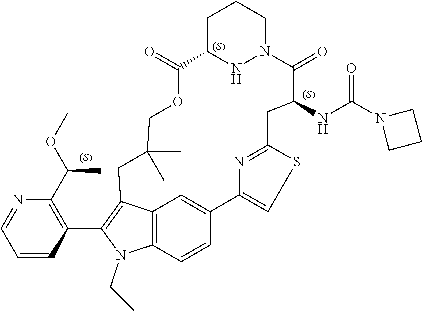 |
| A369 | 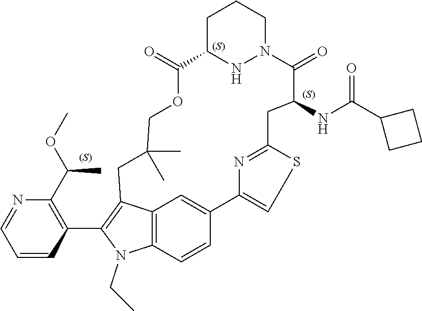 |
| A370 | 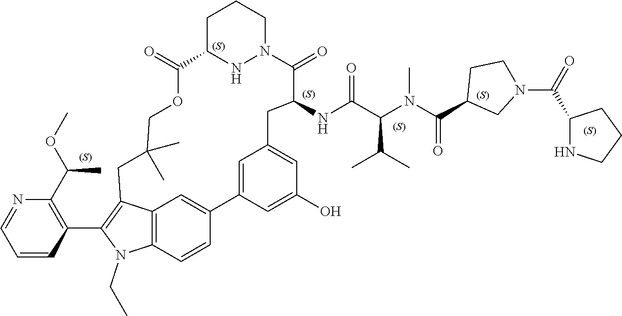 |
| A371 | 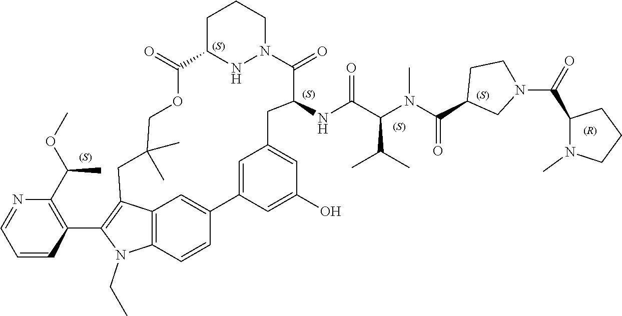 |
| A372 | 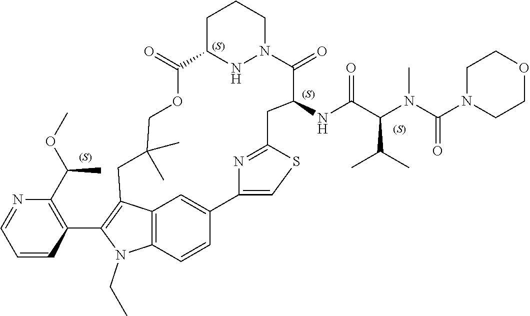 |
| A373 | 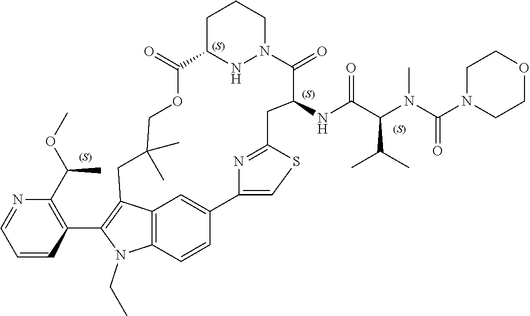 |
| A374 | 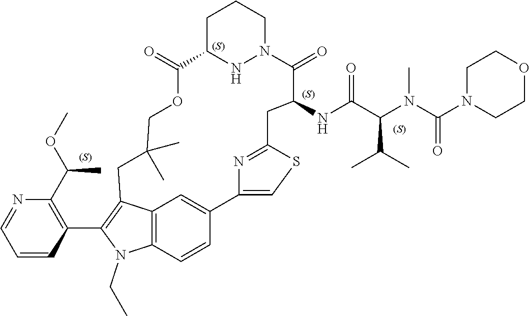 |
| A375 | 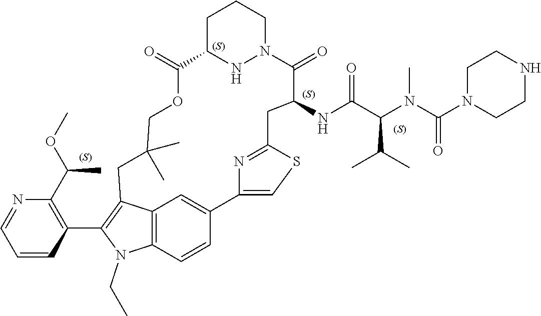 |
| A376 | 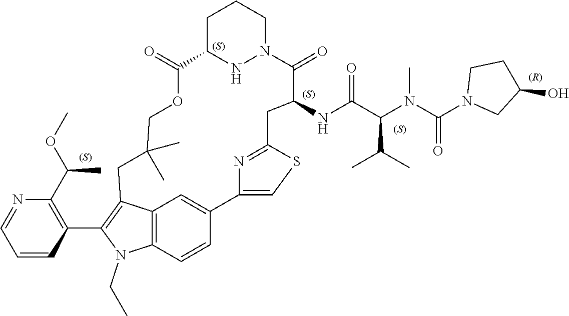 |
| A377 | 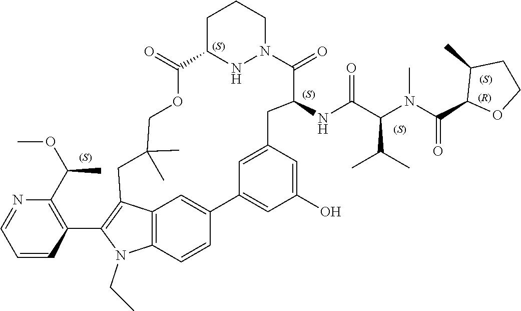 |
| A378 | 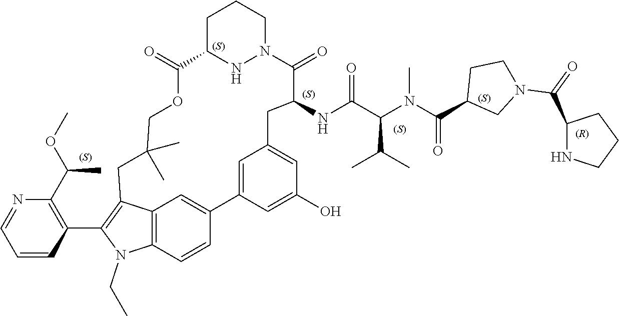 |
| A379 | 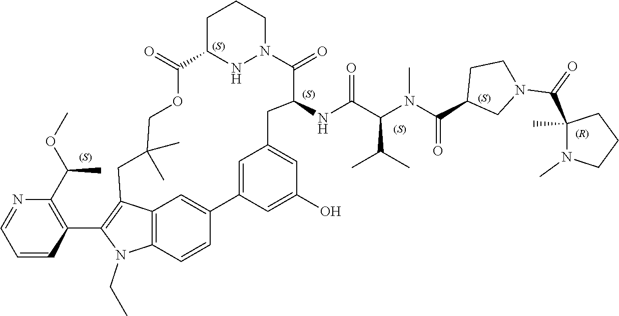 |
| A380 | 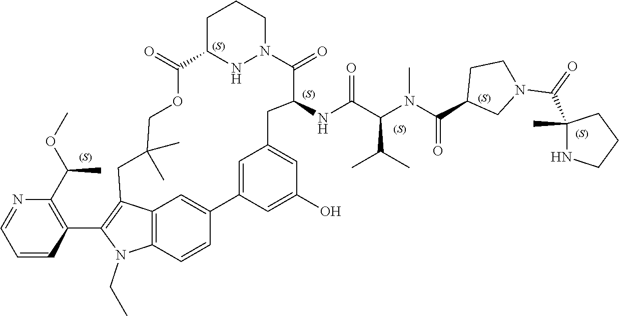 |
| A381 | 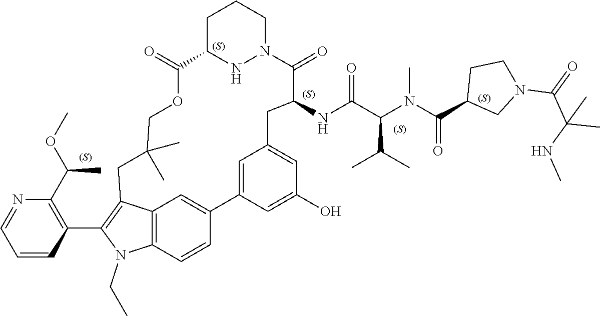 |
| A382 | 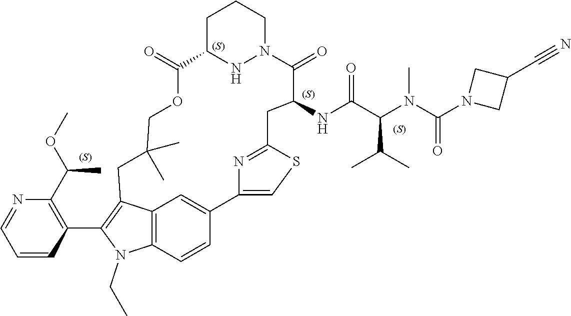 |
| A383 | 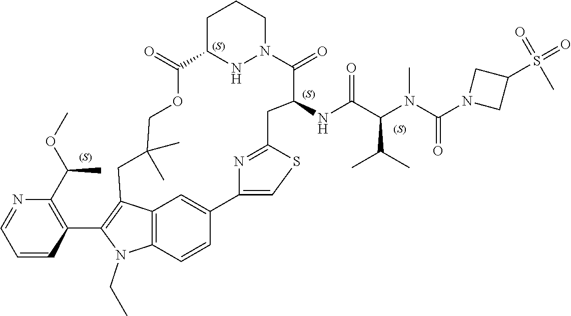 |
| A384 | 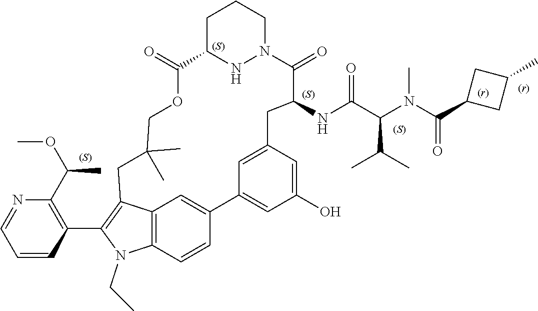 |
| A385 | 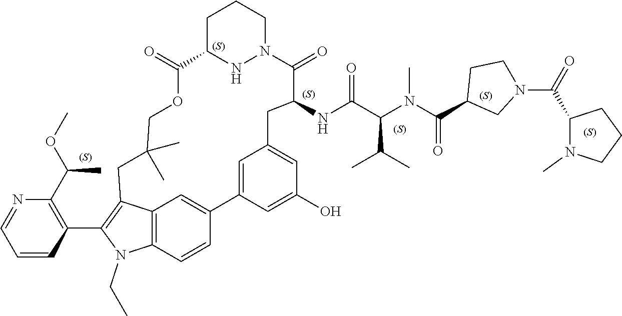 |
| A386 | 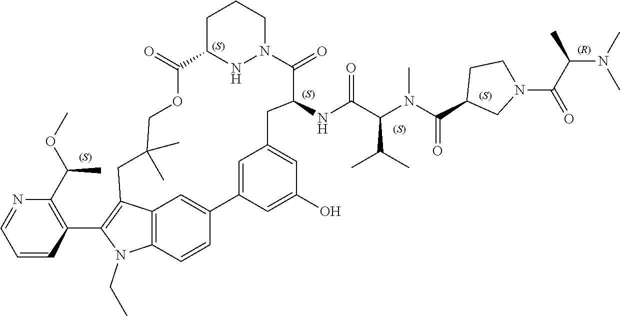 |
| A387 | 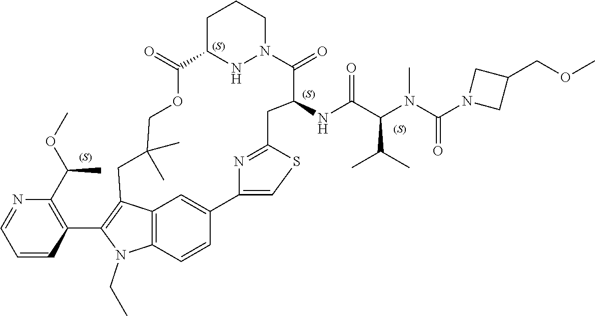 |
| A388 | 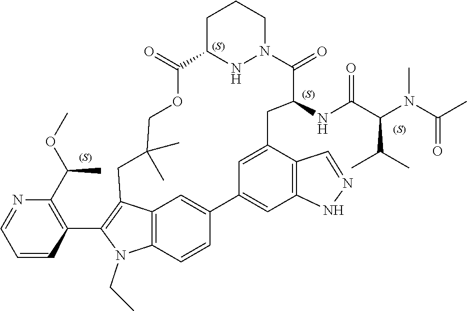 |
| A389 | 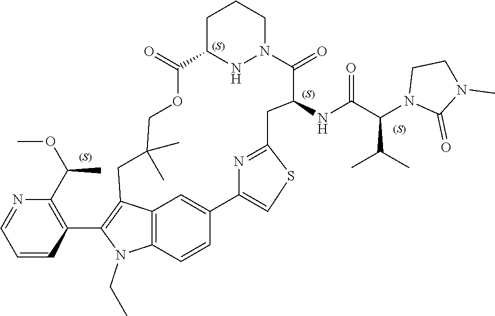 |
| A391 | 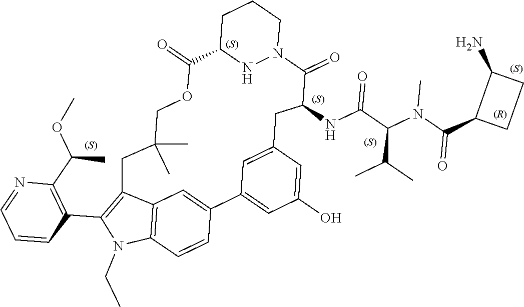 |
| A392 | 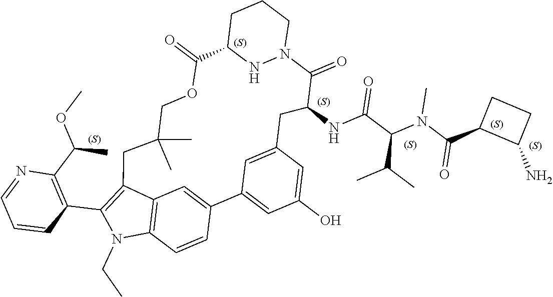 |
| A393 | 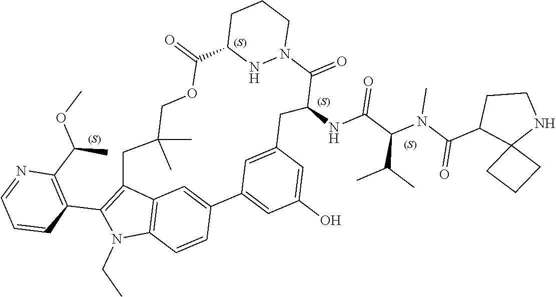 |
| A394 | 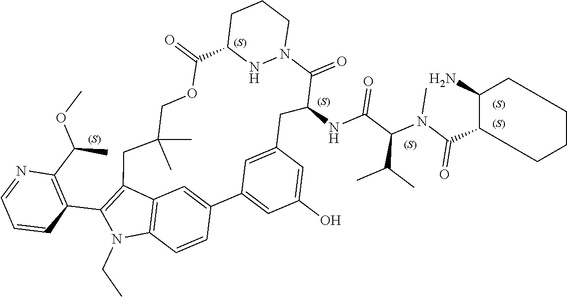 |
| A395 | 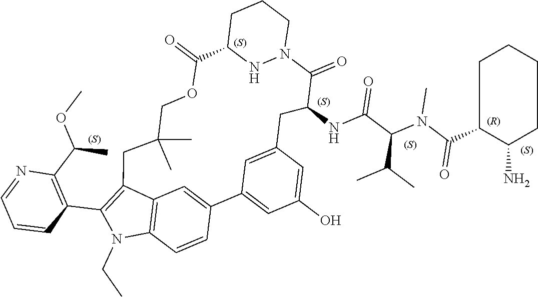 |
| A396 | 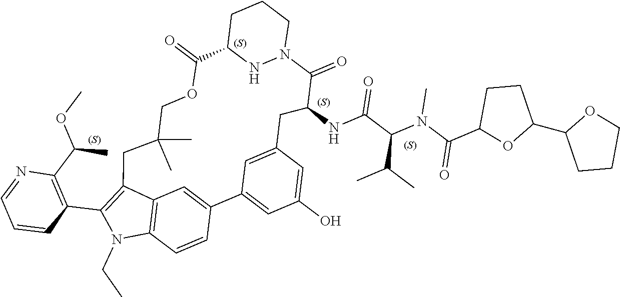 |
| A397 | 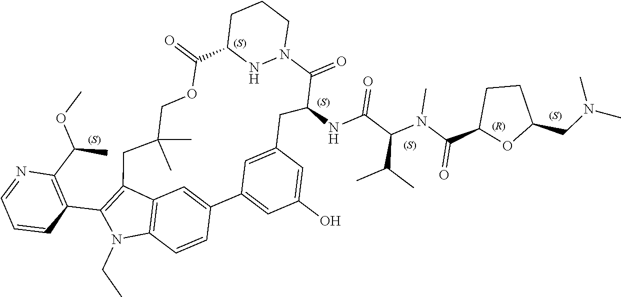 |
| A398 | 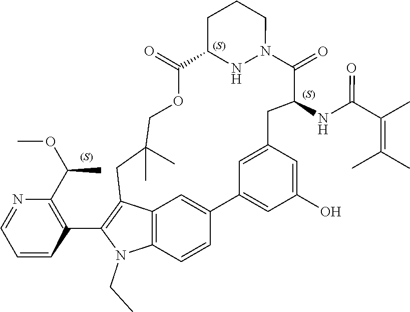 |
| A399 | 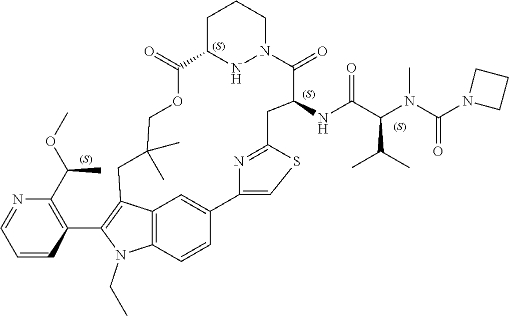 |
| A400 | 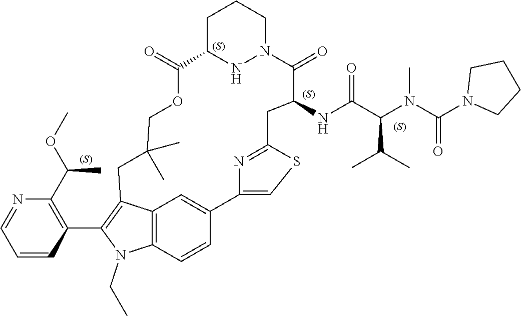 |
| A401 | 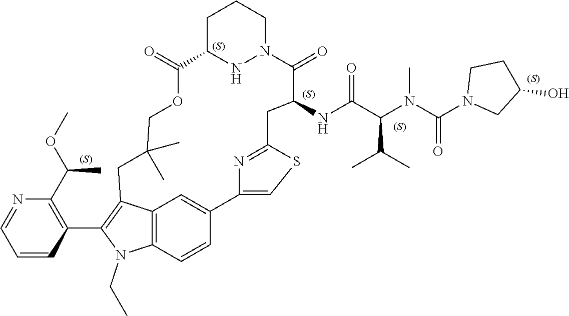 |
| A402 | 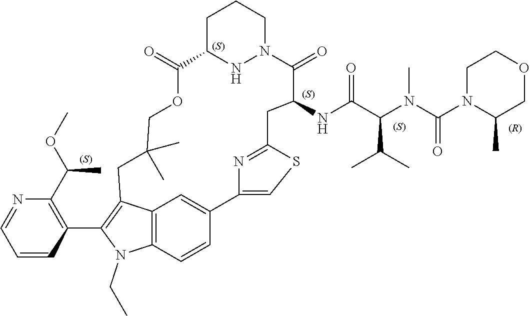 |
| A403 | 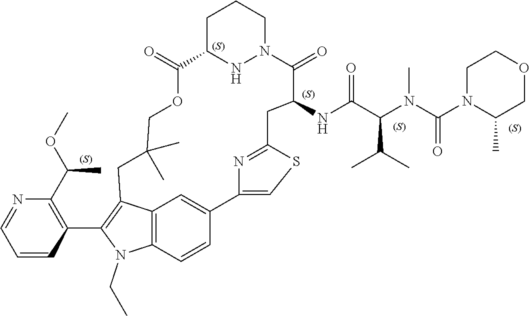 |
| A404 | 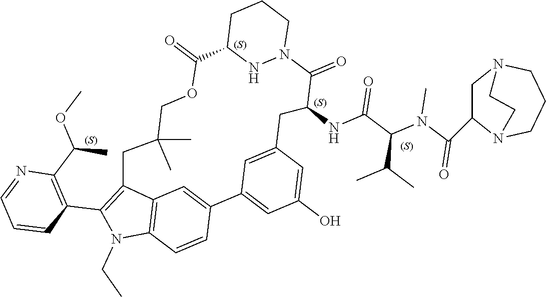 |
| A405 | 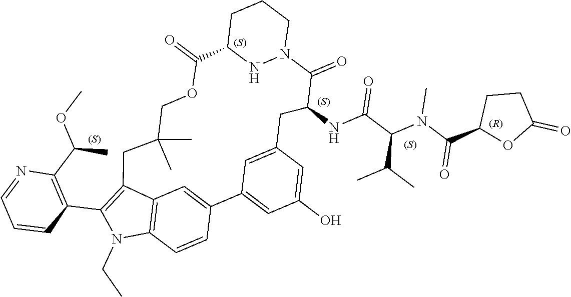 |
| A406 | 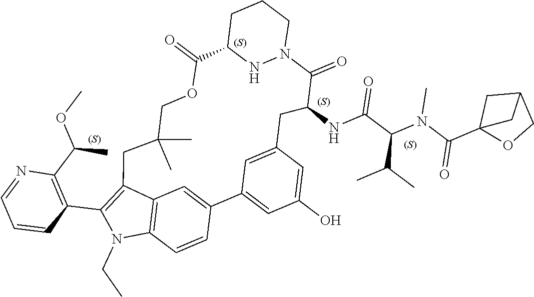 |
| A407 | 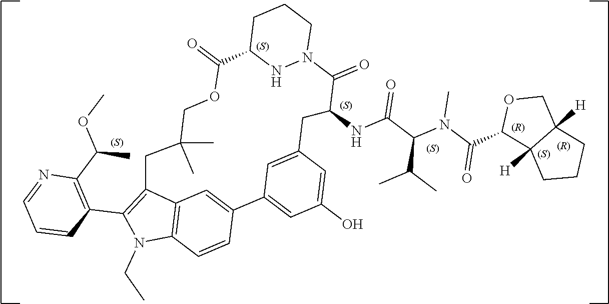 |
| A408 | 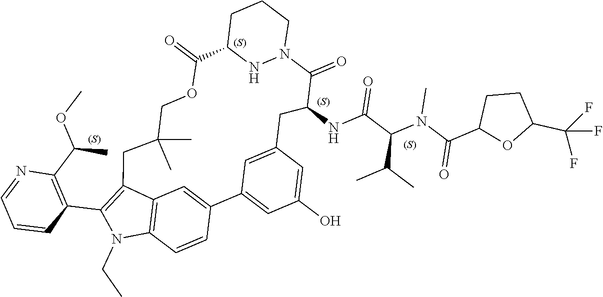 |
| A409 | 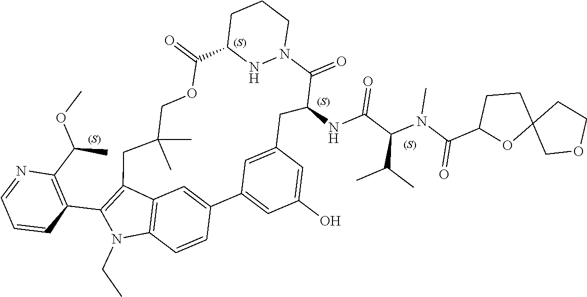 |
| A410 | 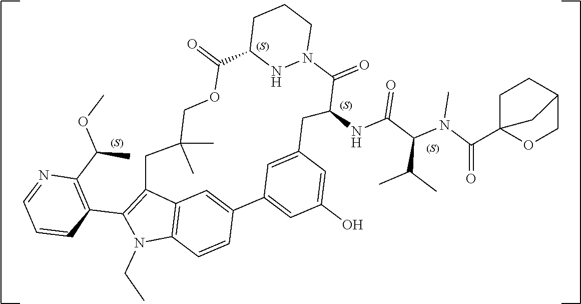 |
| A411 | 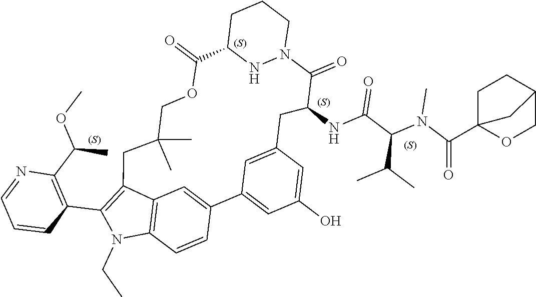 |
| A412 | 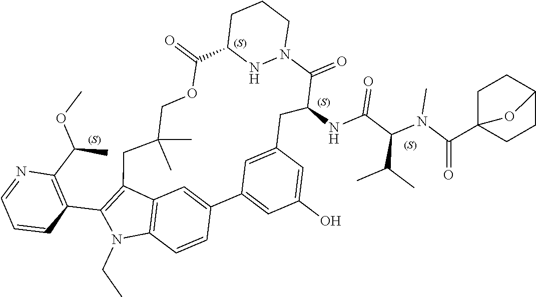 |
| A413 | 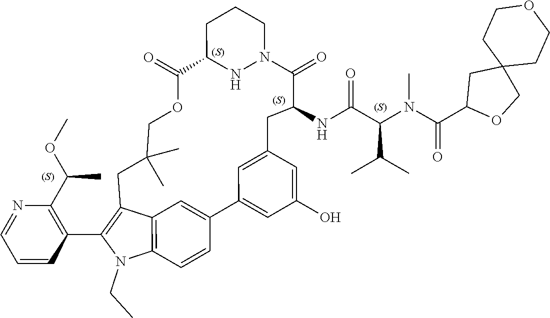 |
| A414 | 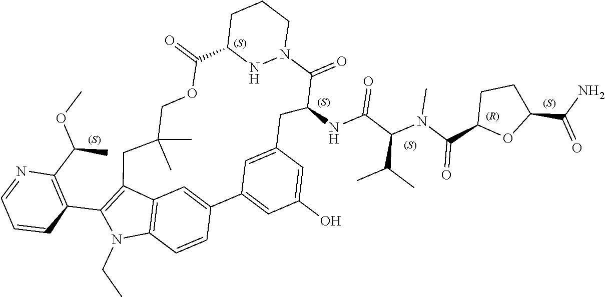 |
| A415 | 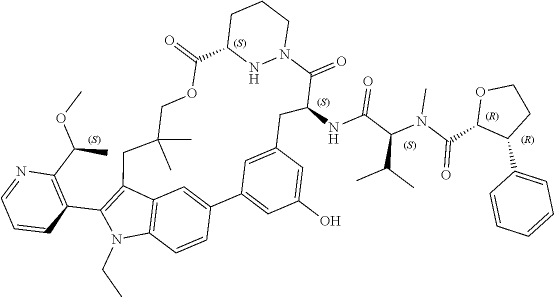 |
| A416 | 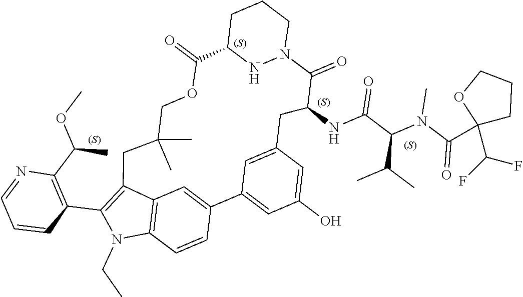 |
| A417 | 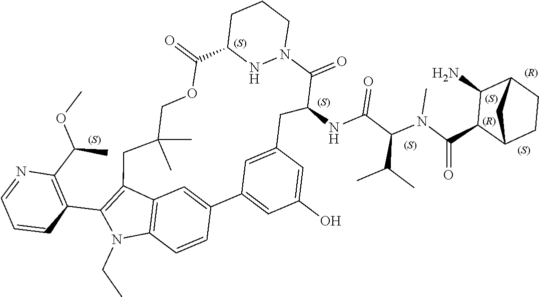 |
| A418 | 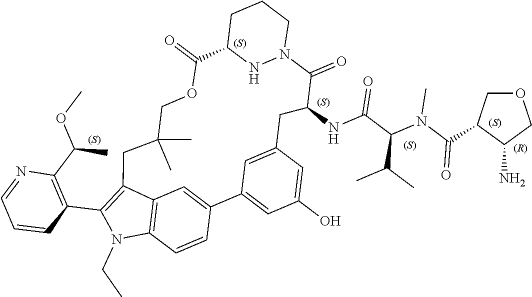 |
| A419 | 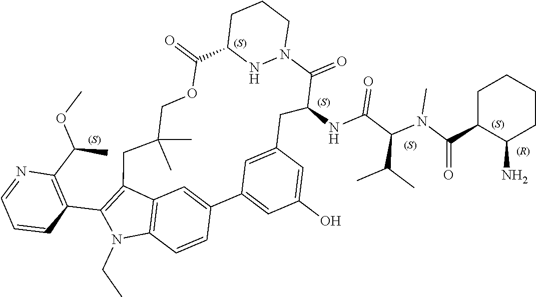 |
| A420 | 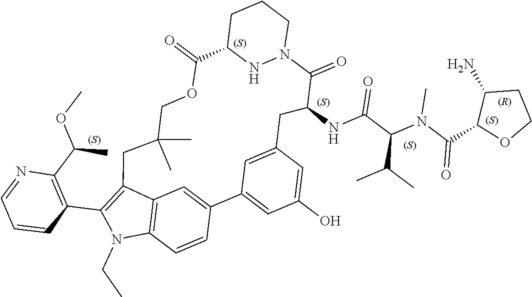 |
| A421 | 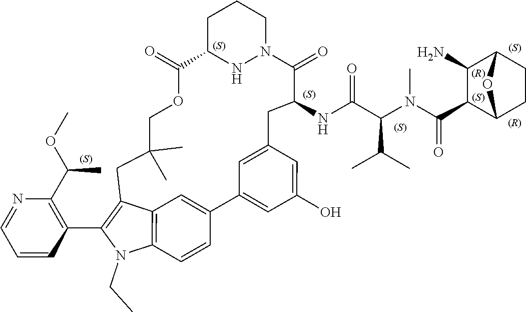 |
| A422 | 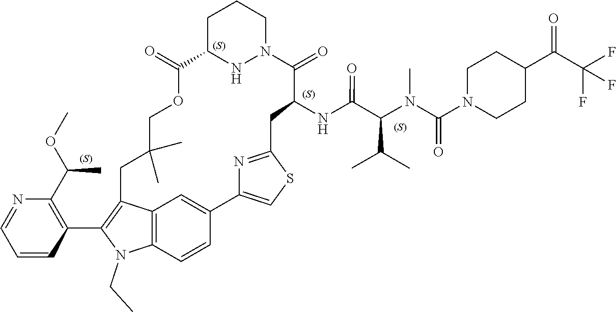 |
| A423 | 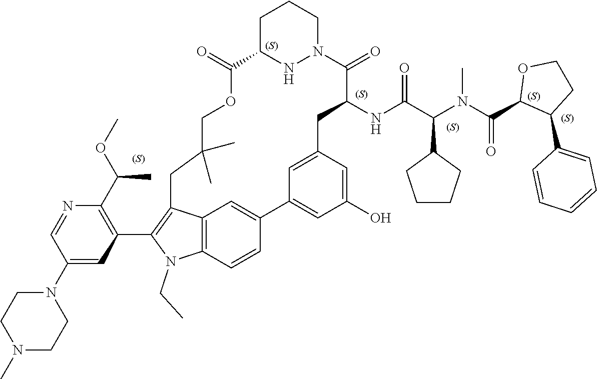 |
| A424 | 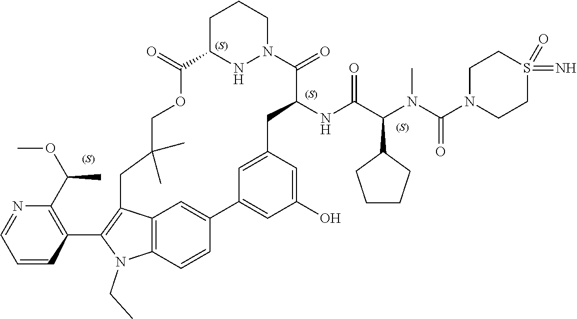 |
| A425 | 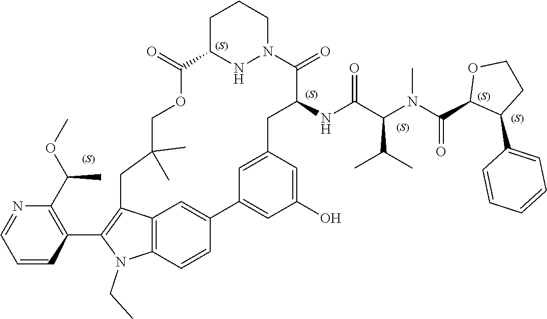 |
| A426 | 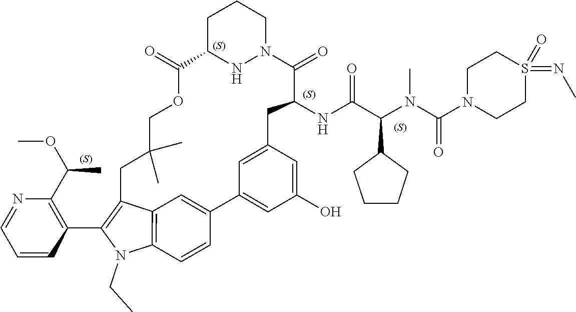 |
| A427 | 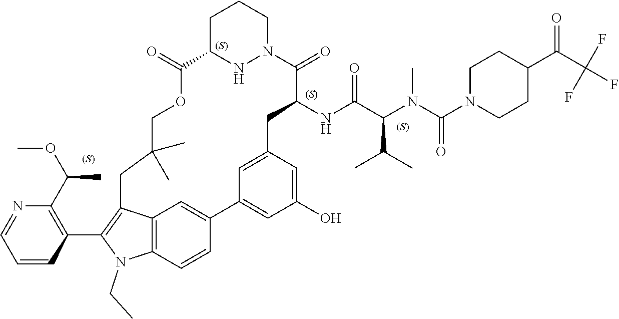 |
| A428 | 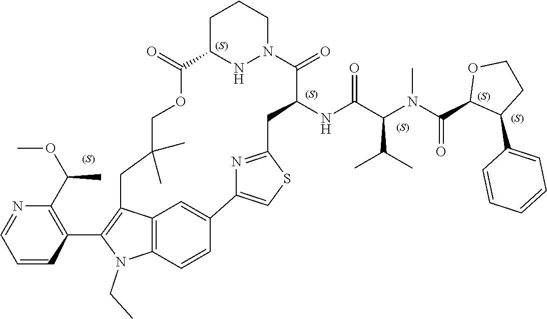 |
| A429 | 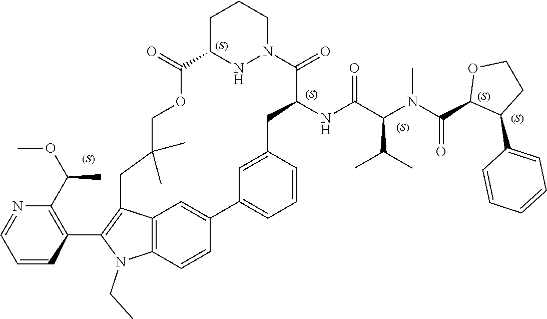 |
| A430 | 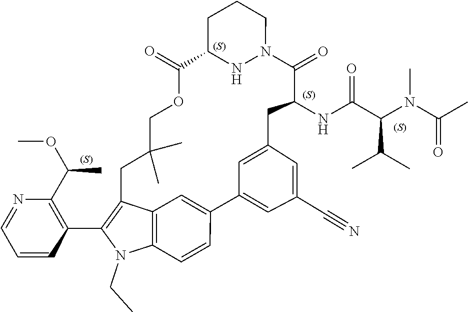 |
| A431 | 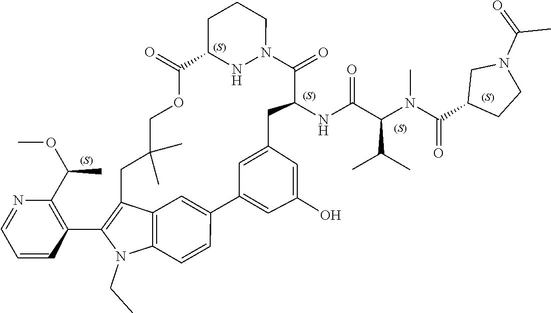 |
| A432 | 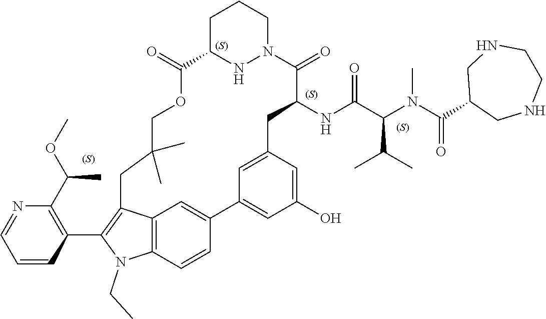 |
| A433 | 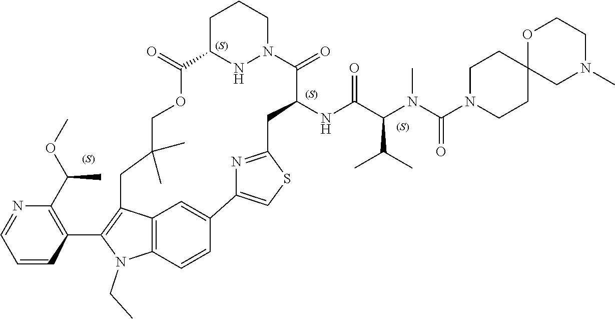 |
| A434 | 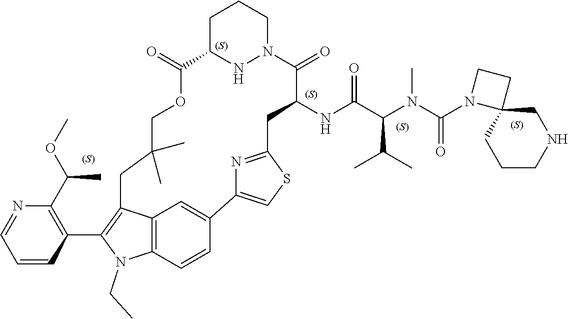 |
| A435 | 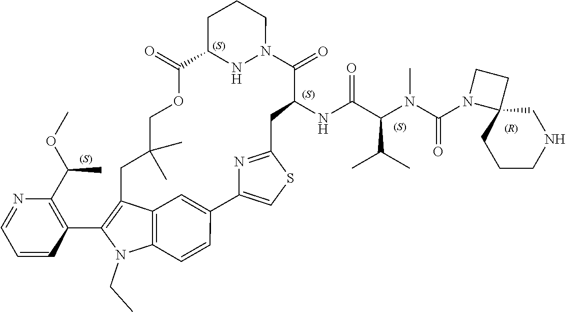 |
| A436 | 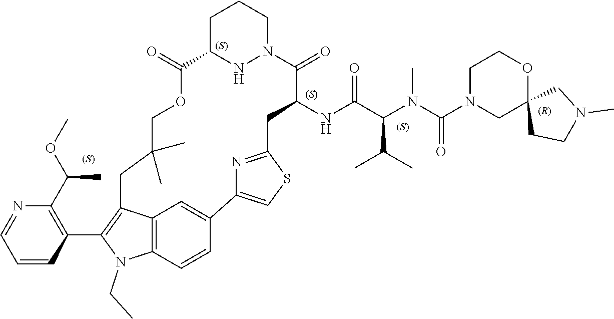 |
| A437 | 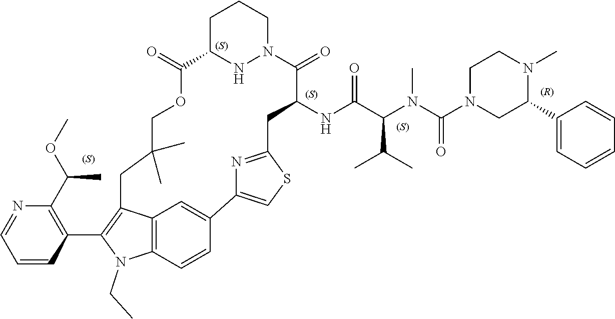 |
| A438 | 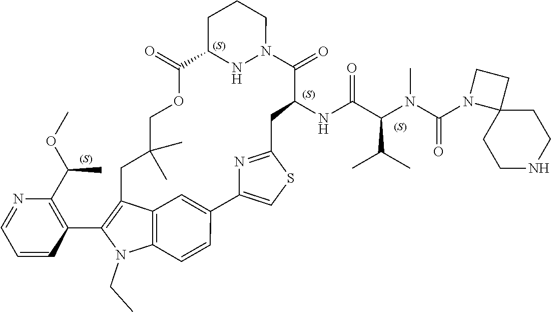 |
| A439 | 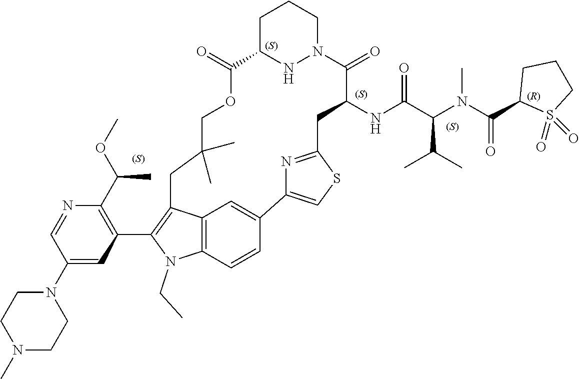 |
| A440 | 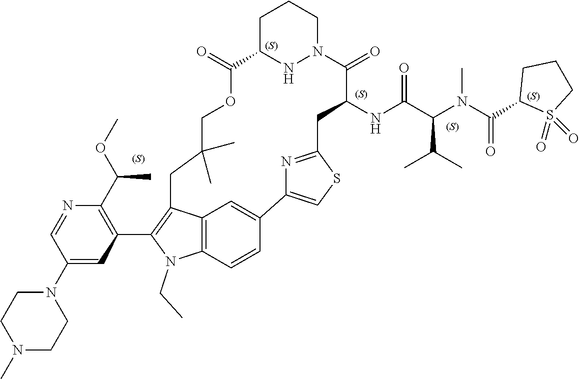 |
| A441 | 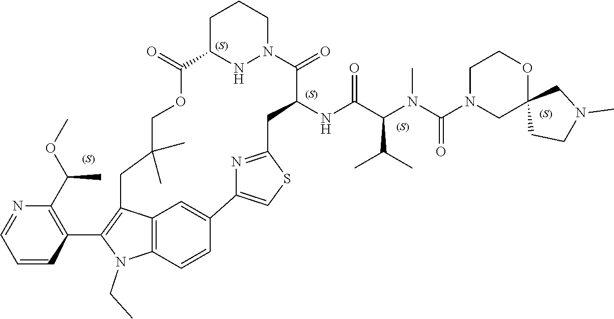 |
| A442 | 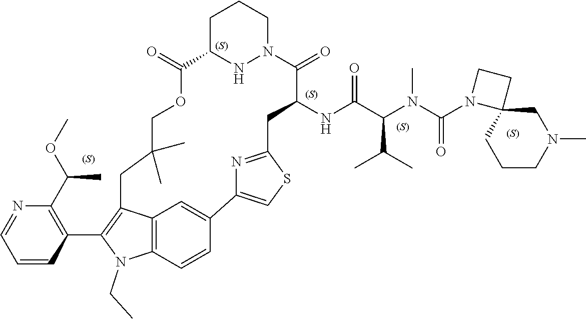 |
| A443 | 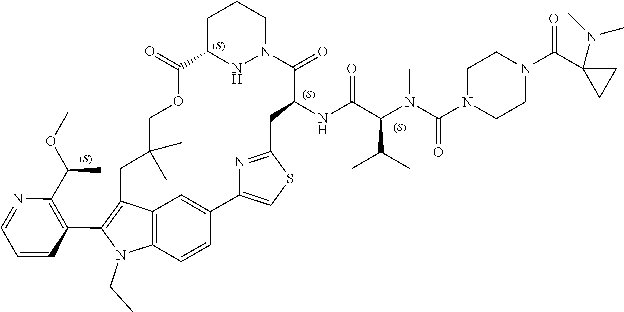 |
| A444 | 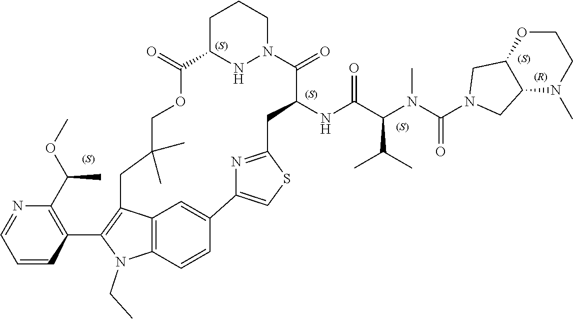 |
| A445 | 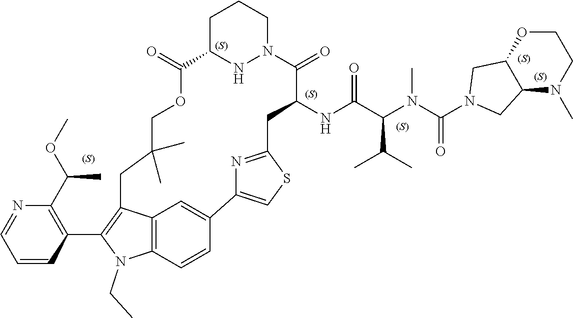 |
| A446 | 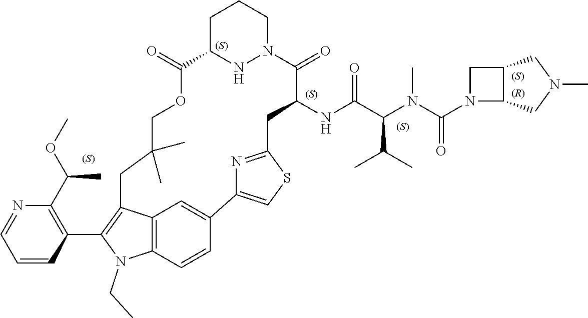 |
| A447 | 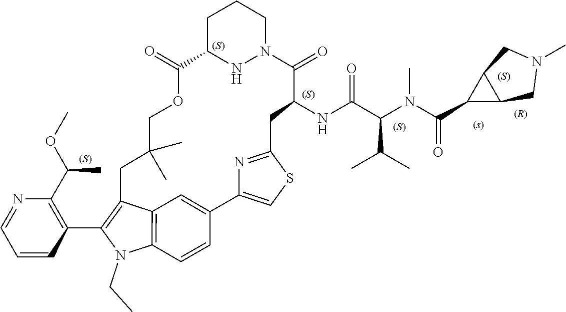 |
| A448 | 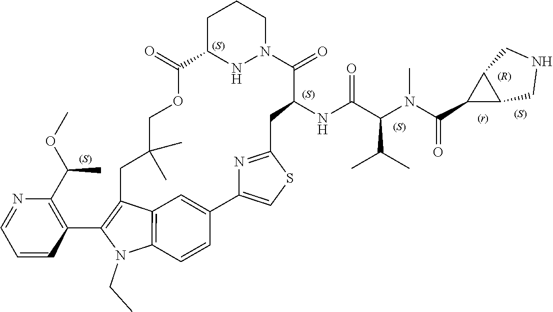 |
| A449 | 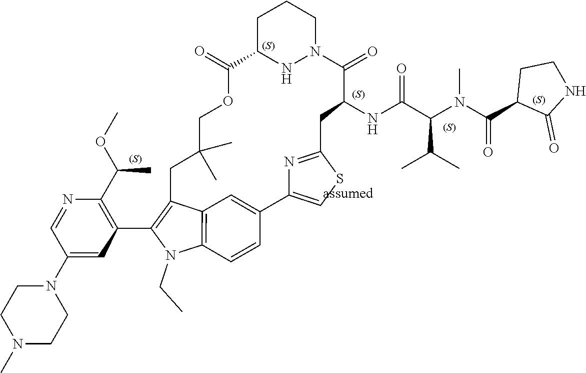 |
| A450 | 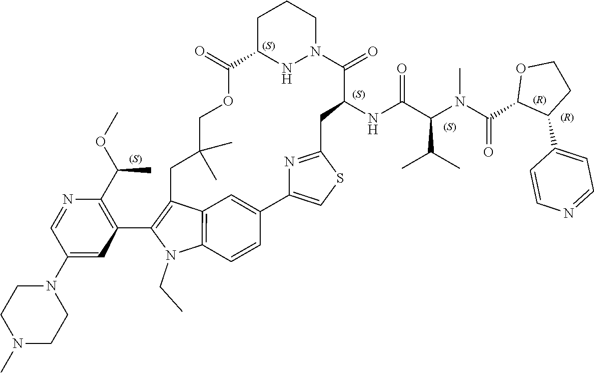 |
| A451 | 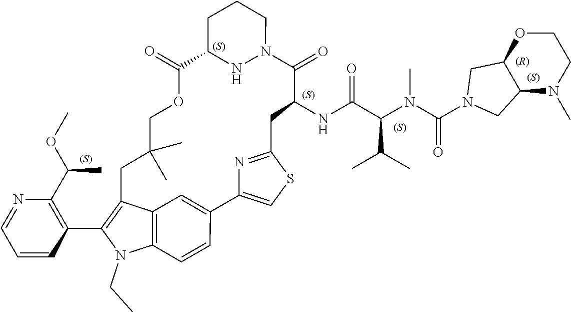 |
| A452 | 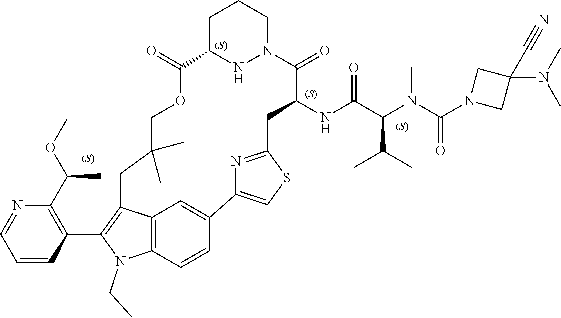 |
| A453 | 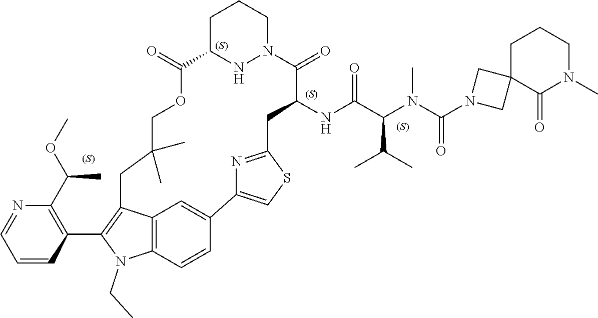 |
| A454 | 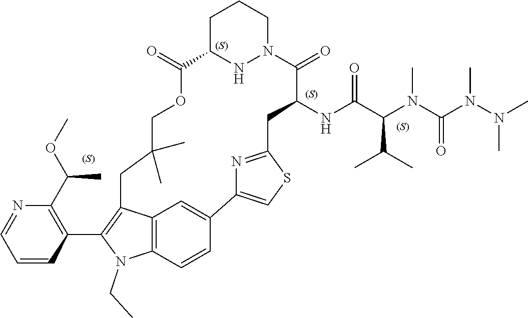 |
| A455 | 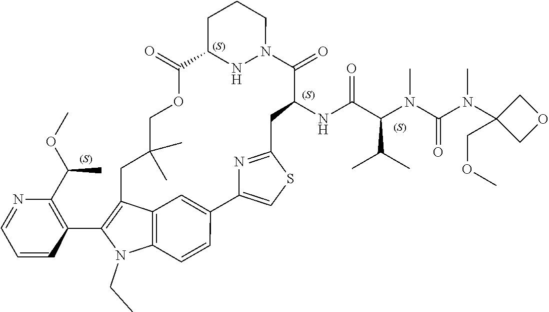 |
| A456 | 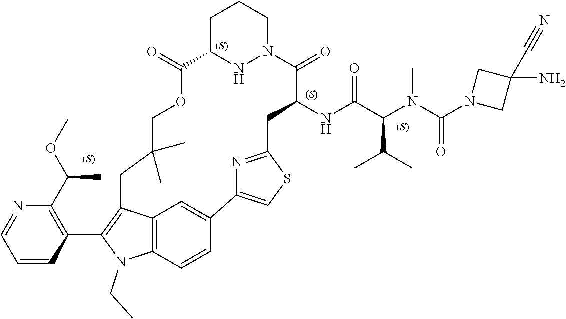 |
| A457 | 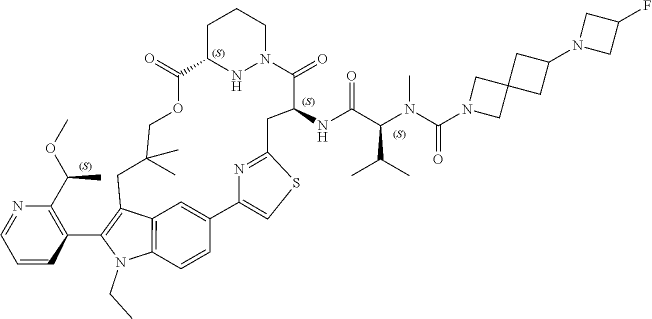 |
| A458 | 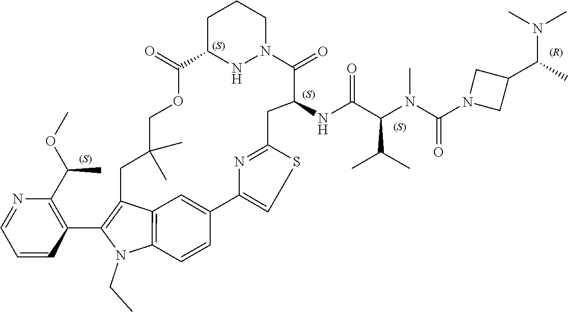 |
| A459 | 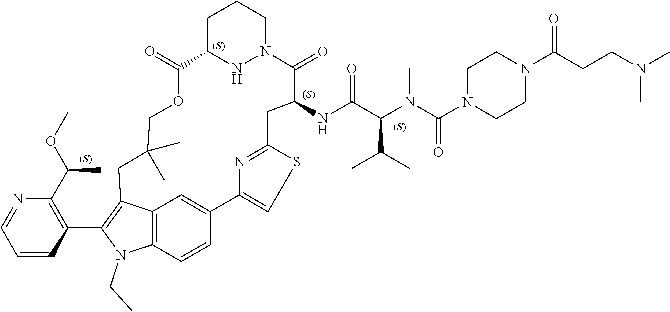 |
| A460 | 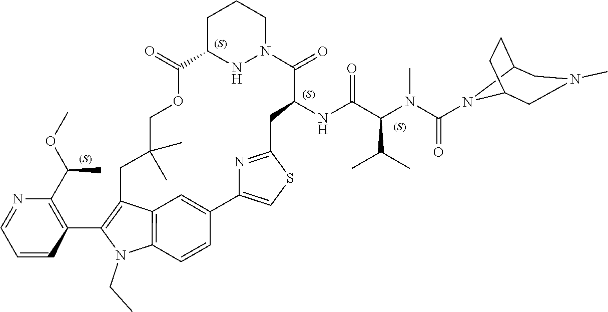 |
| A461 | 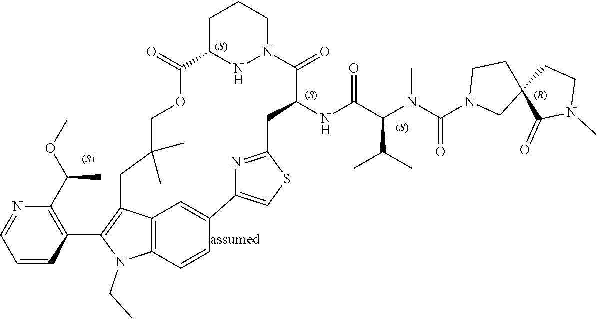 |
| A462 | 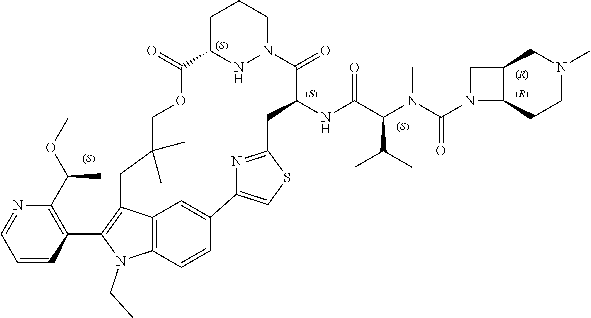 |
| A463 | 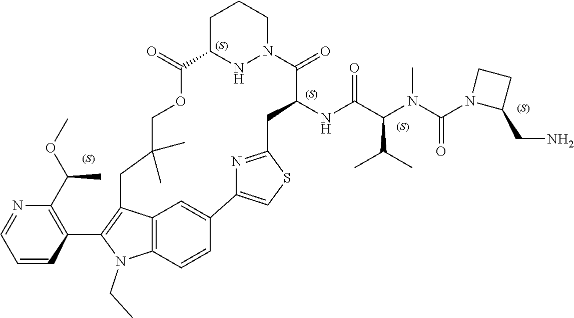 |
| A464 | 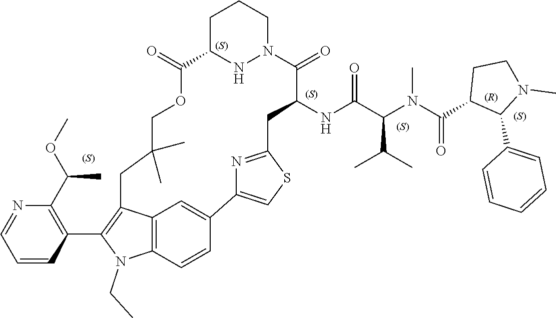 |
| A465 | 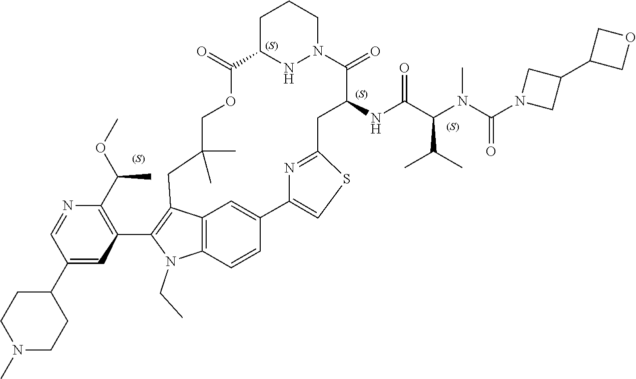 |
| A466 | 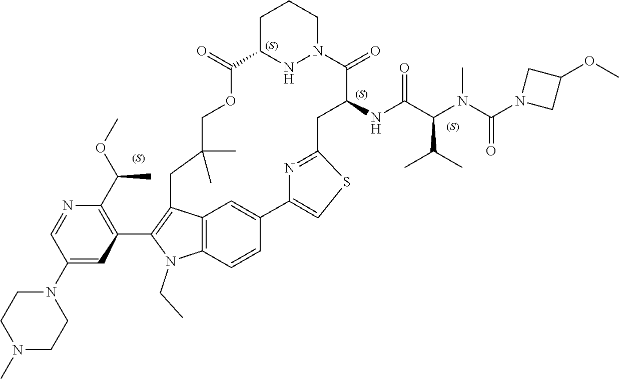 |
| A467 | 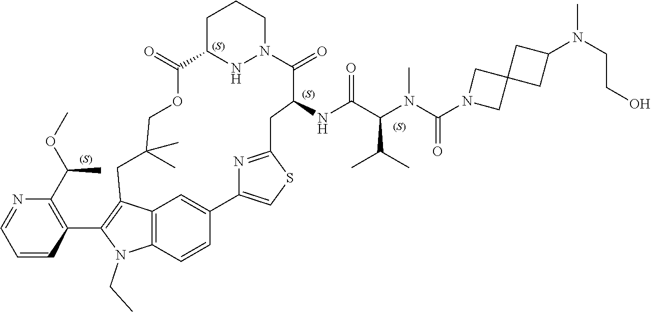 |
| A468 | 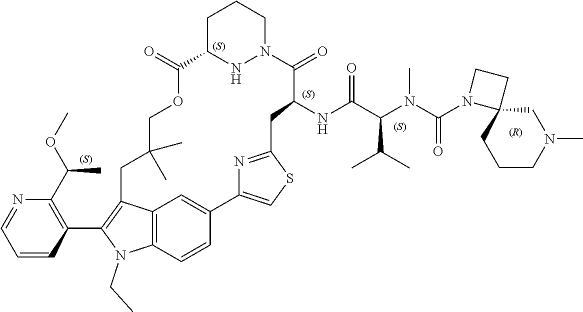 |
| A469 | 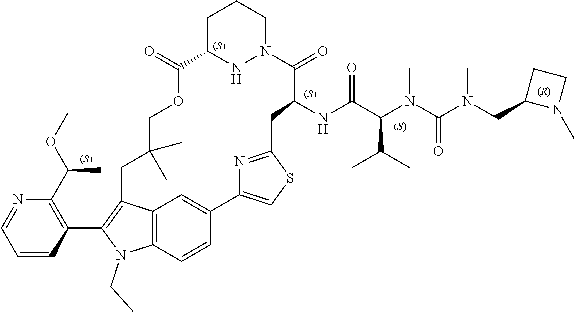 |
| A470 | 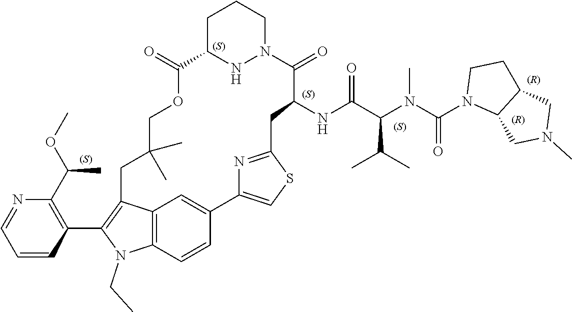 |
| A471 | 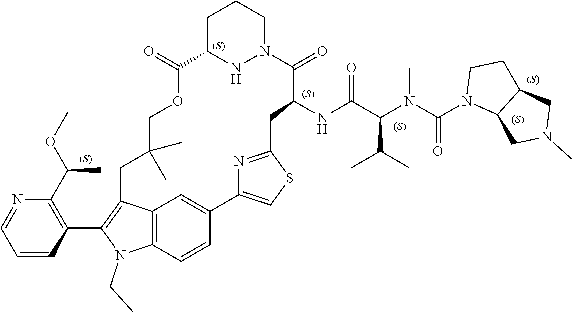 |
| A472 | 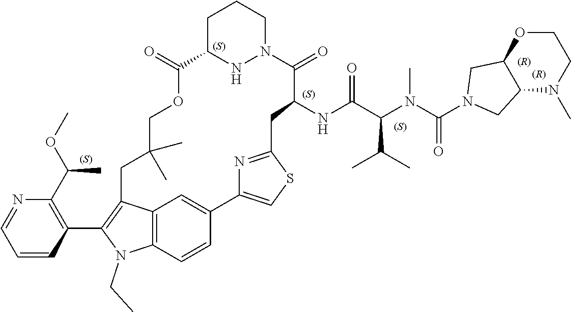 |
| A473 | 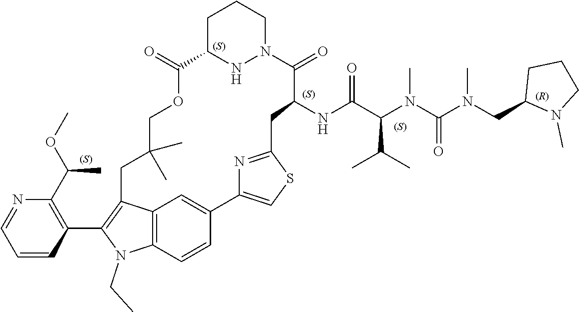 |
| A474 | 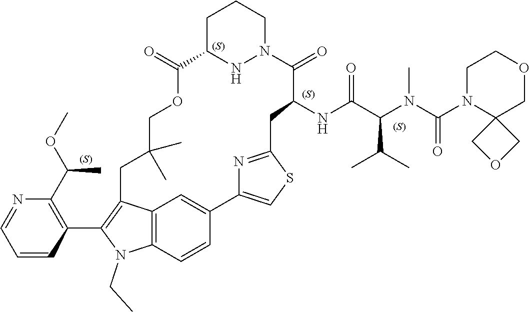 |
| A475 | 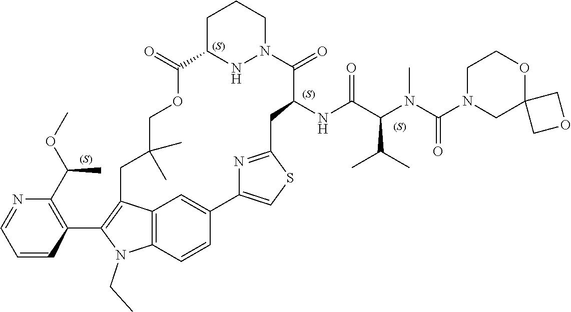 |
| A476 | 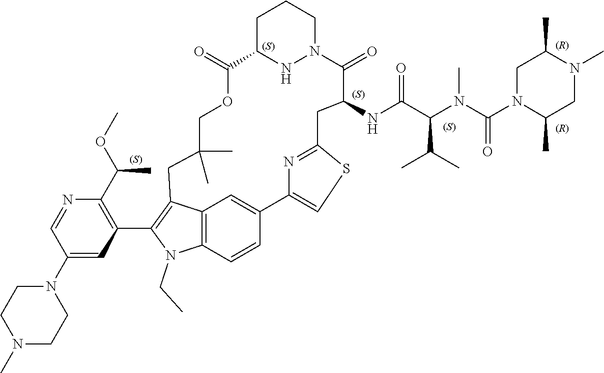 |
| A477 | 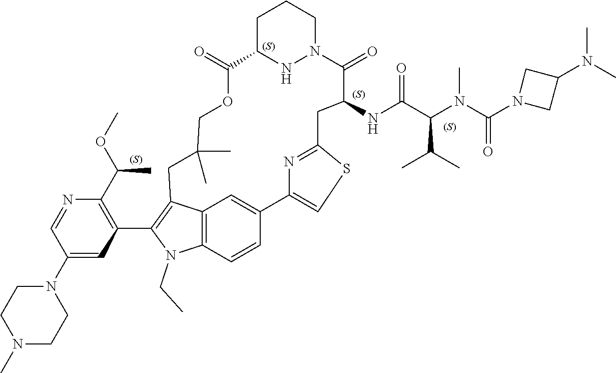 |
| A478 | 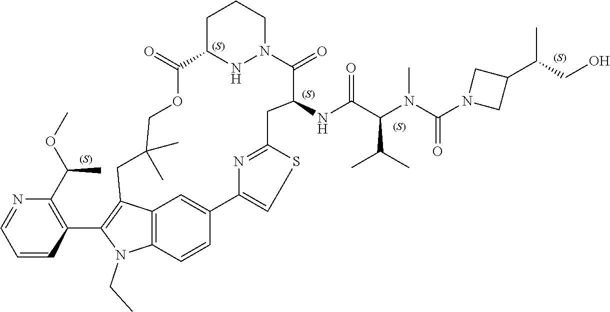 |
| A479 | 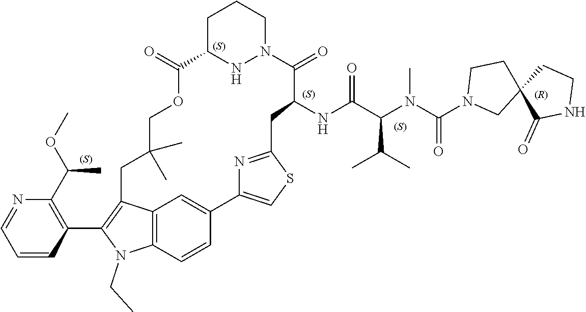 |
| A480 | 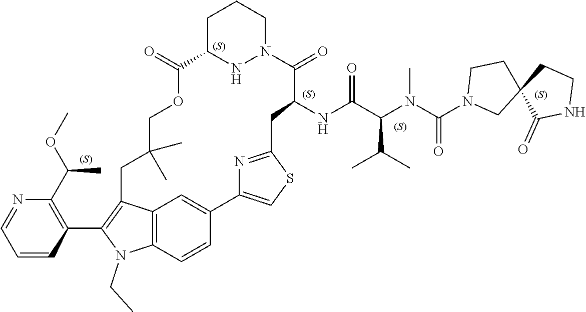 |
| A481 | 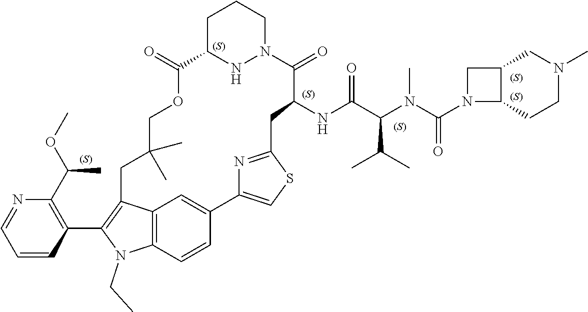 |
| A482 | 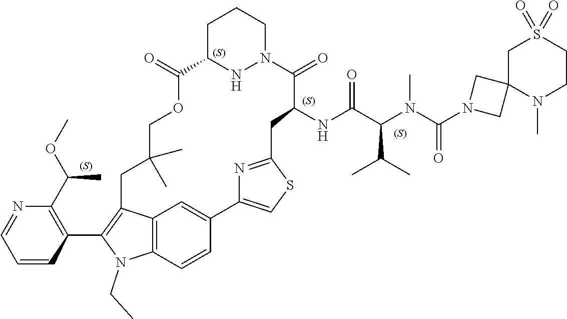 |
| A483 | 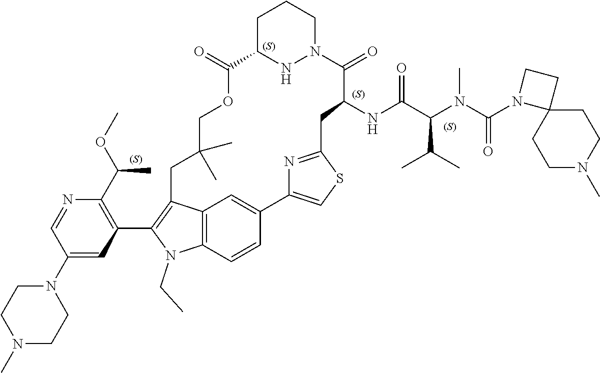 |
| A484 | 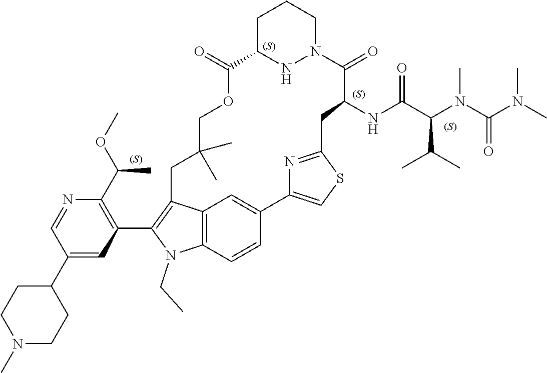 |
| A485 | 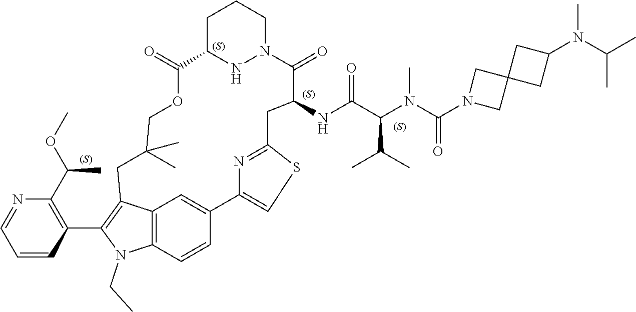 |
| A486 | 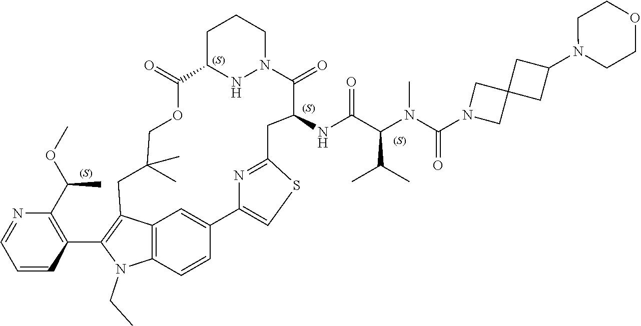 |
| A487 | 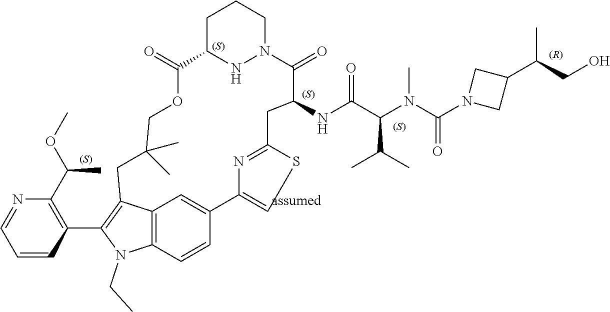 |
| A488 | 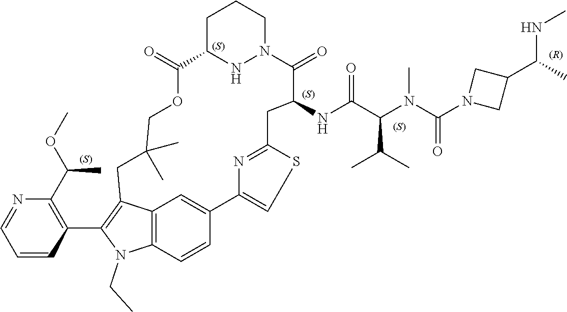 |
| A489 | 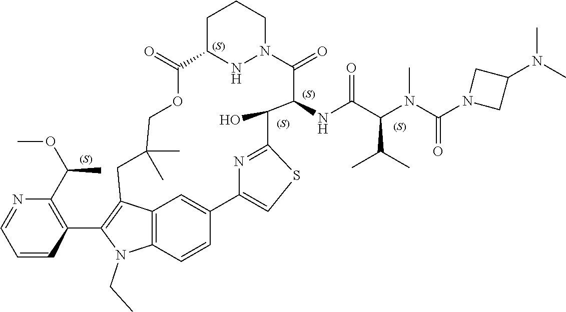 |
| A490 | 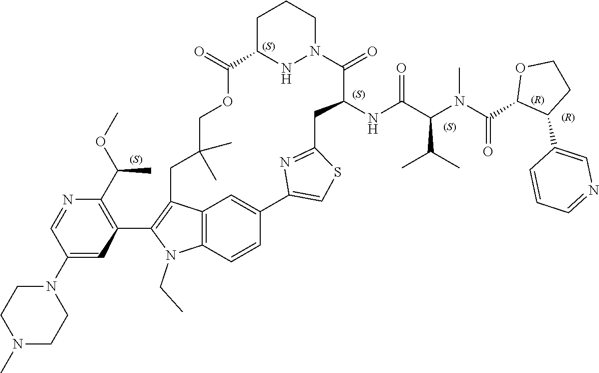 |
| A491 | 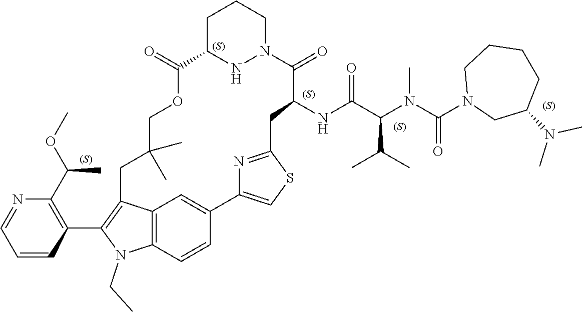 |
| A492 | 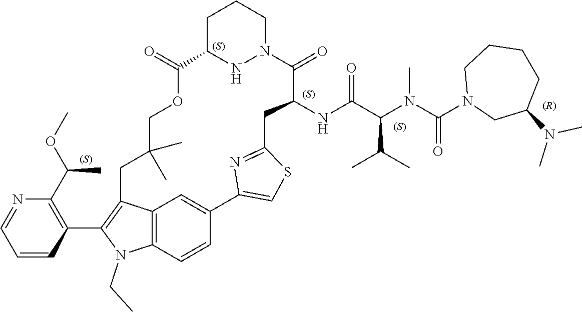 |
| A493 | 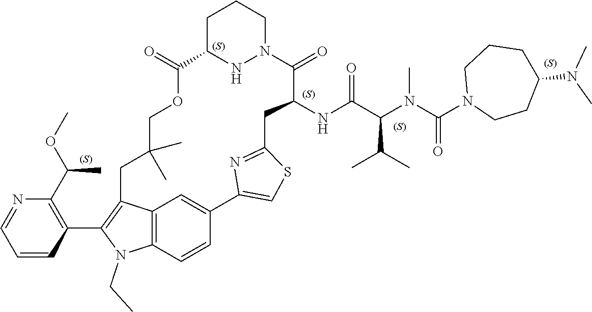 |
| A494 | 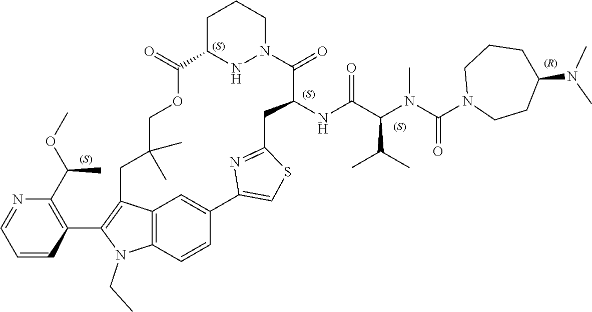 |
| A495 | 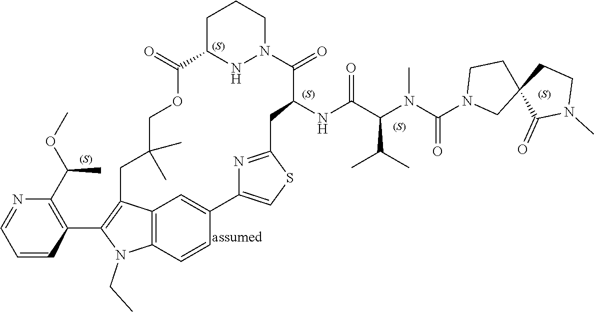 |
| A496 | 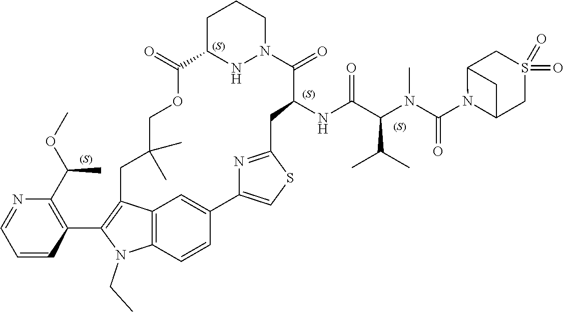 |
| A497 | 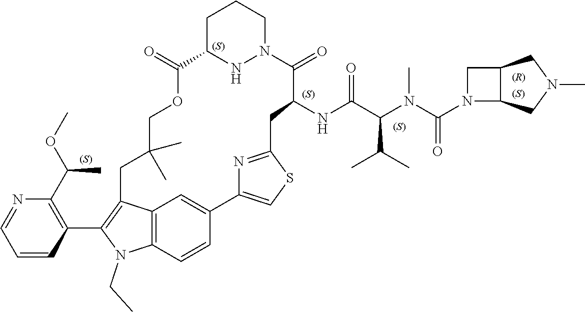 |
| A498 | 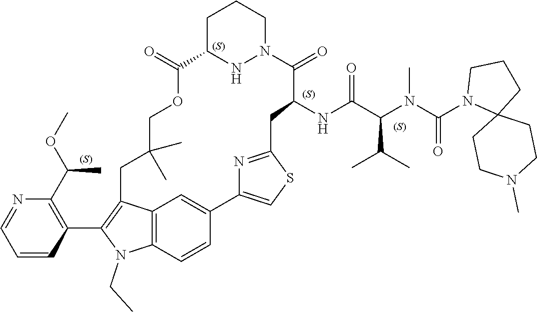 |
| A499 | 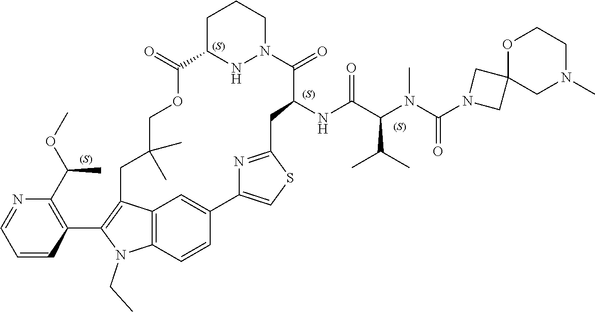 |
| A500 | 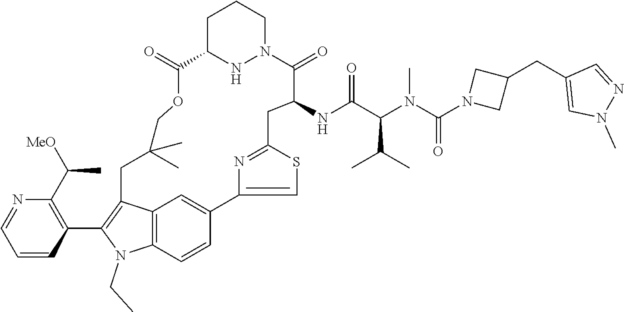 |
| A501 | 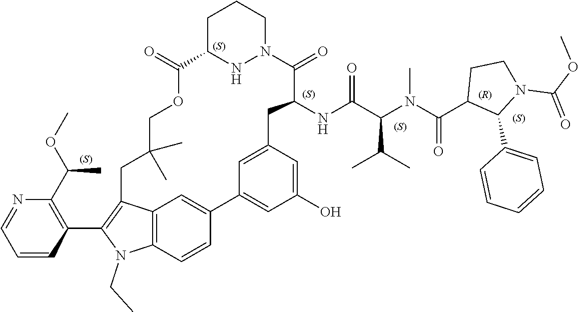 |
| A502 | 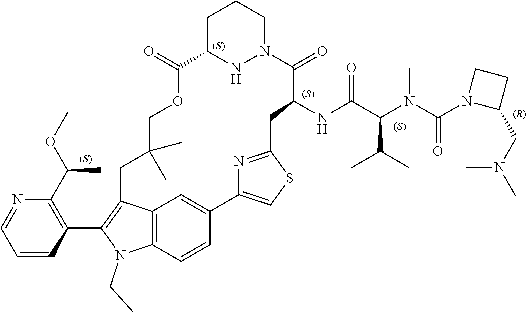 |
| A503 | 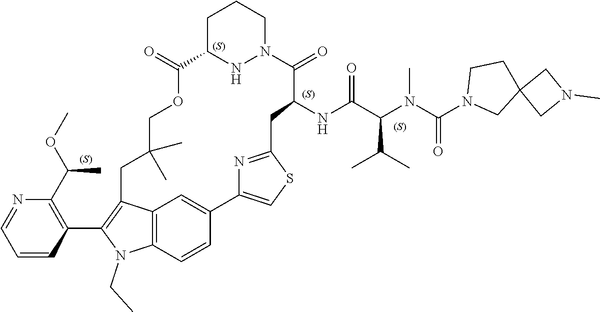 |
| A504 | 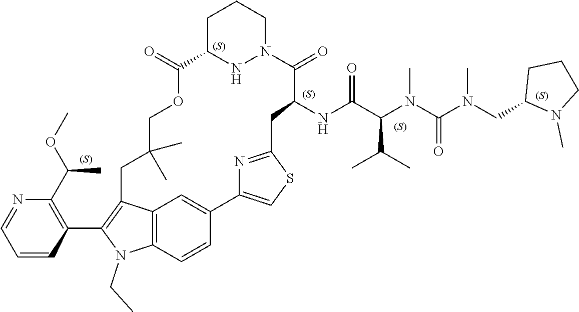 |
| A505 | 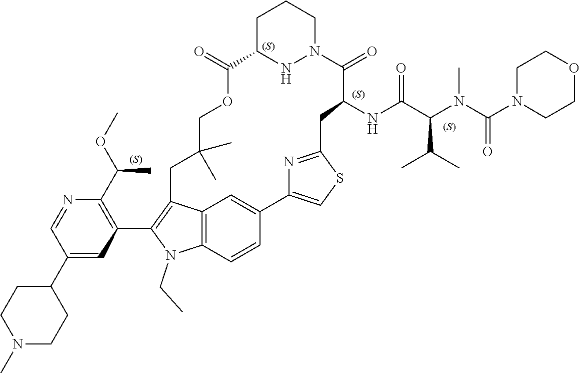 |
| A506 | 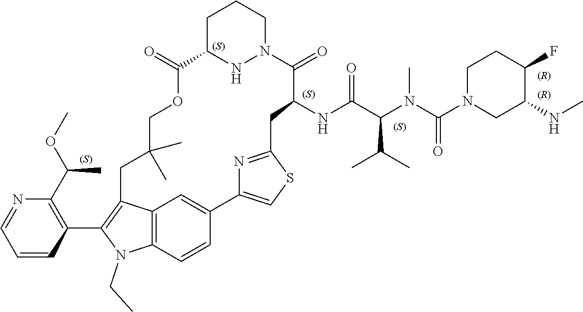 |
| A507 | 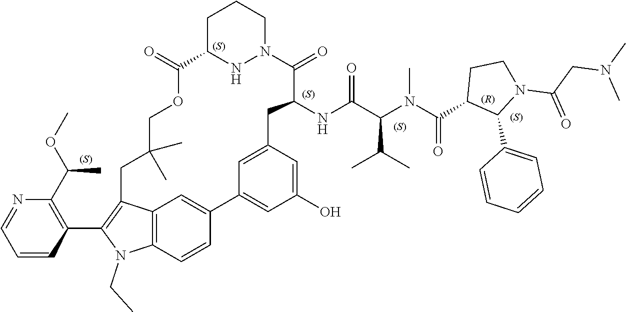 |
| A508 | 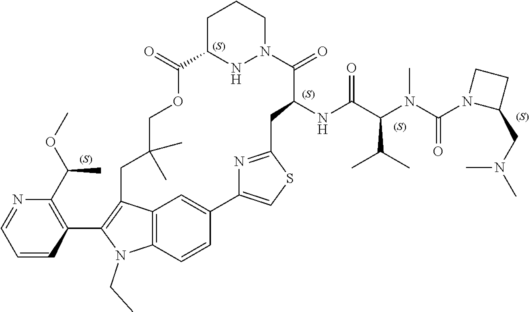 |
| A509 | 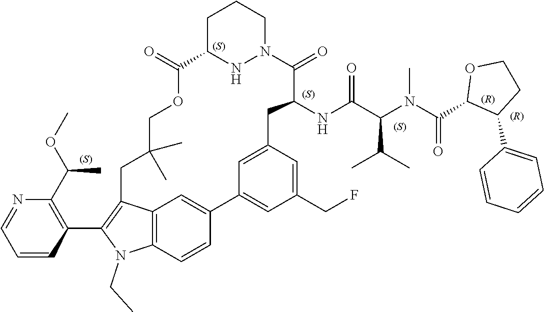 |
| A510 | 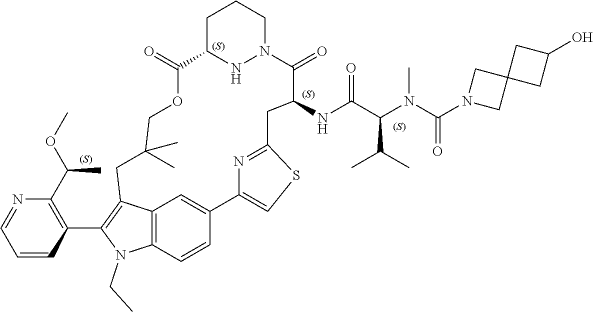 |
| A511 | 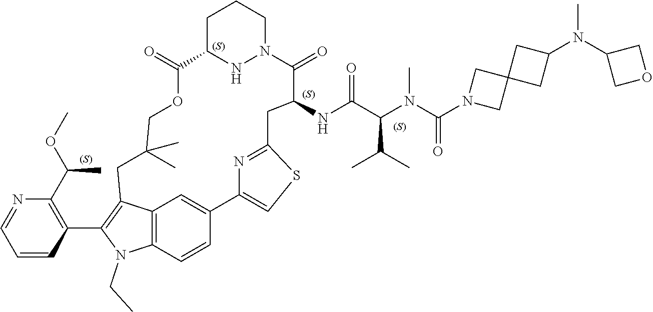 |
| A512 | 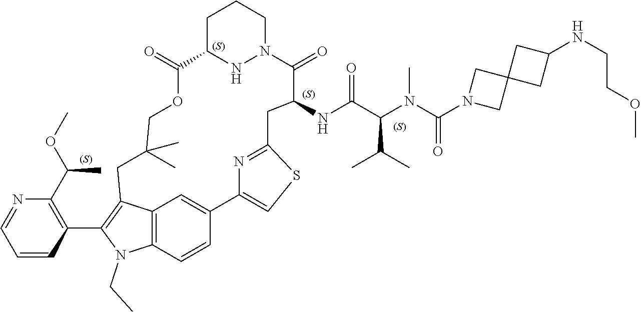 |
| A513 | 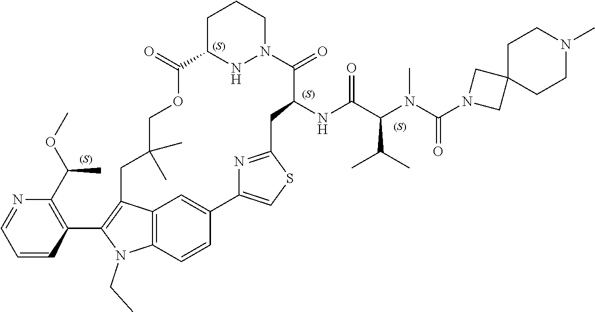 |
| A514 | 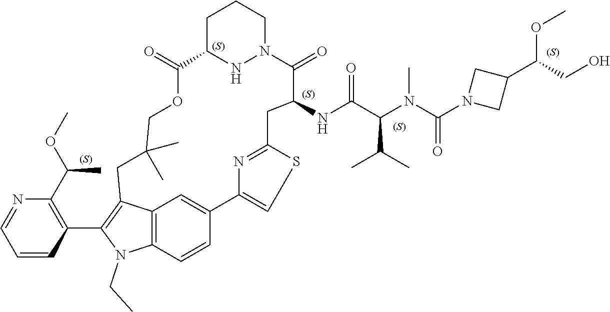 |
| A515 | 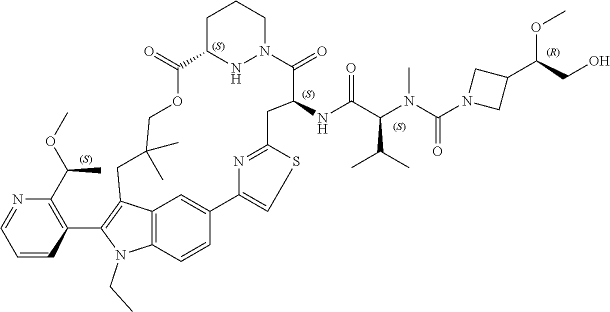 |
| A516 | 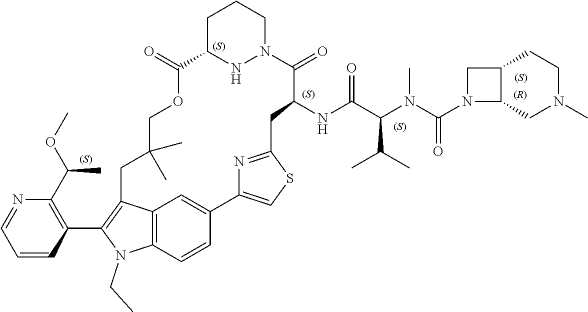 |
| A517 | 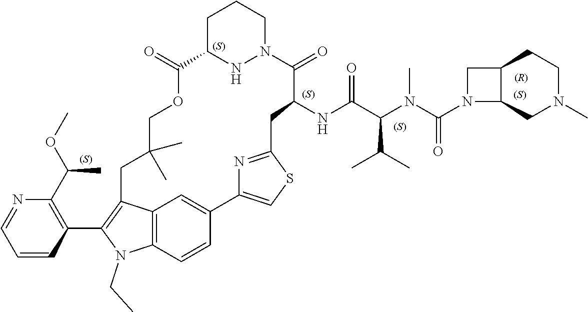 |
| A518 | 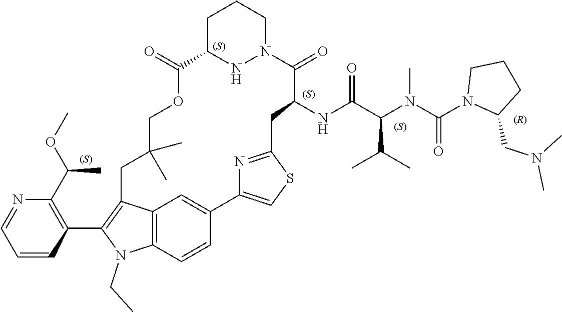 |
| A519 | 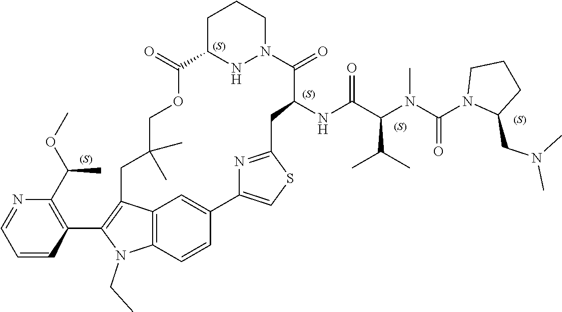 |
| A520 | 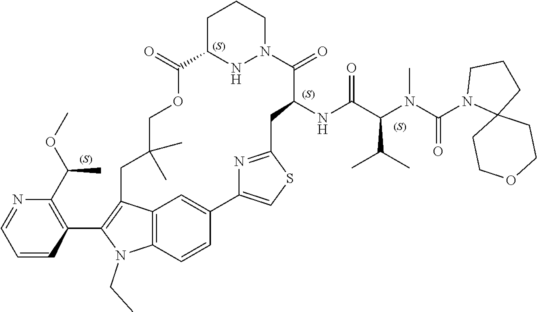 |
| A521 | 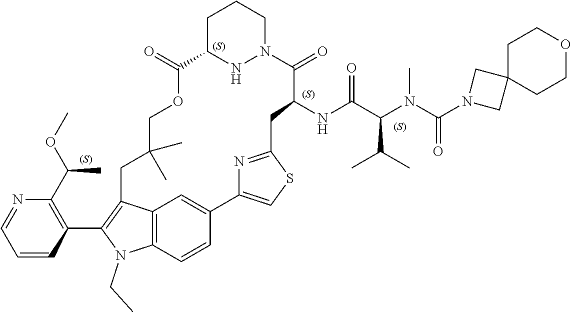 |
| A522 | 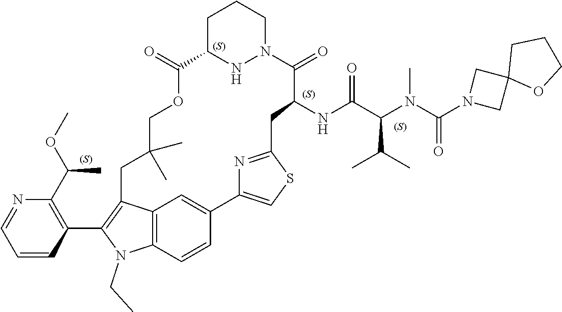 |
| A523 | 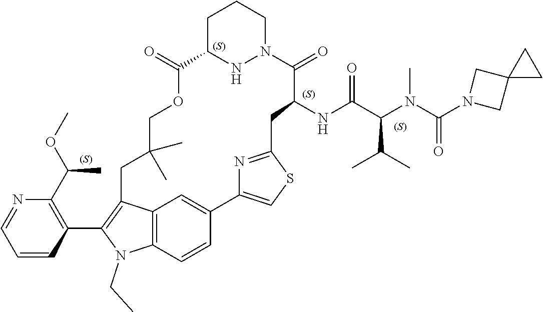 |
| A524 | 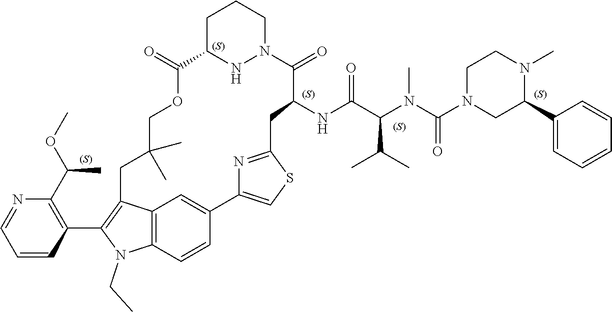 |
| A525 | 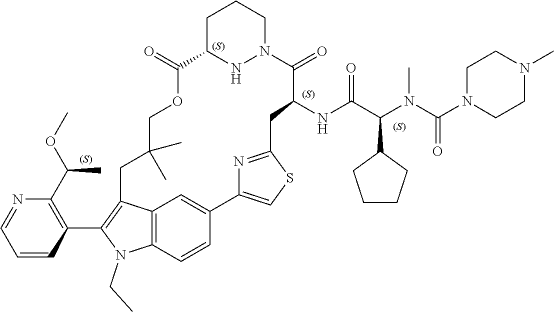 |
| A526 | 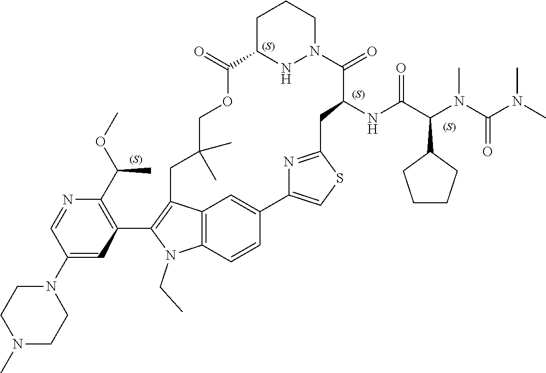 |
| A527 | 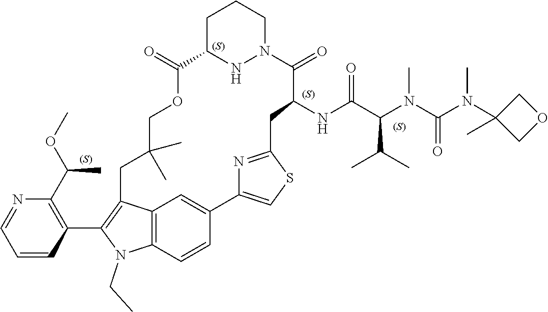 |
| A528 | 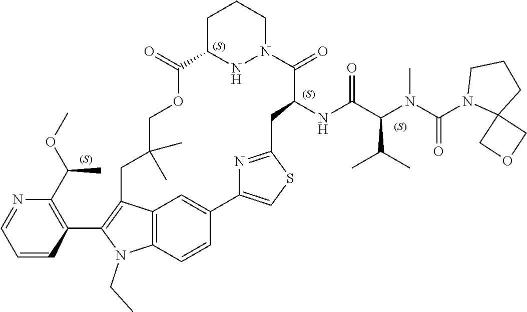 |
| A529 | 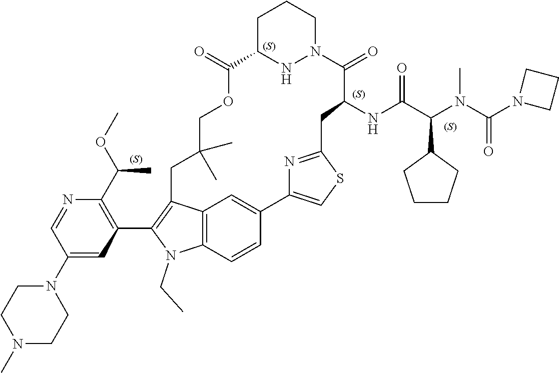 |
| A530 | 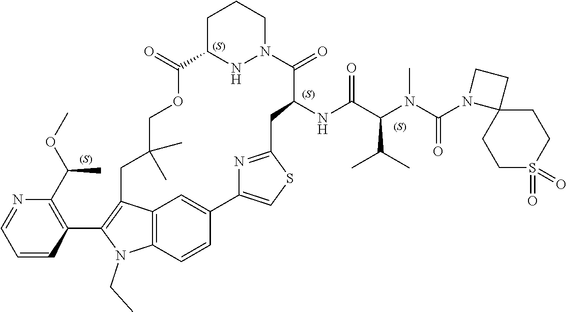 |
| A531 | 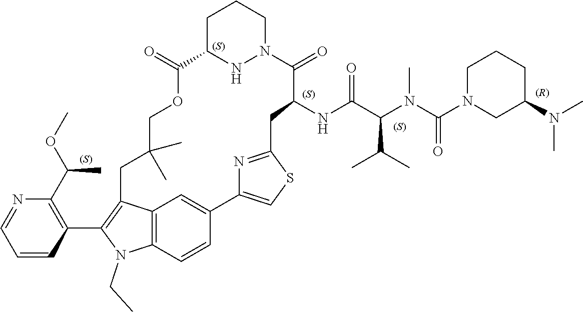 |
| A532 | 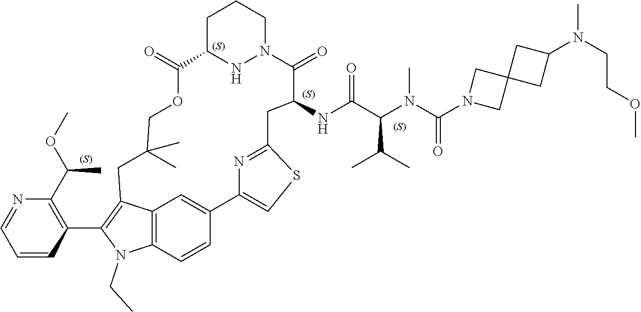 |
| A533 | 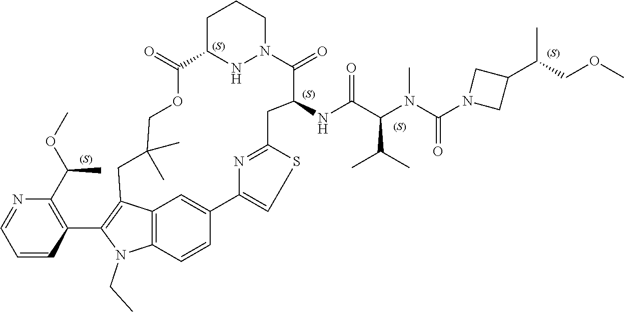 |
| A534 | 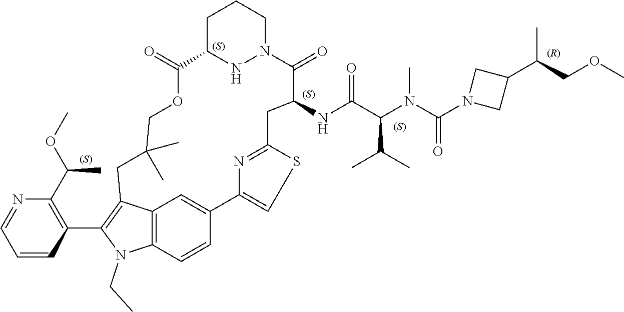 |
| A535 | 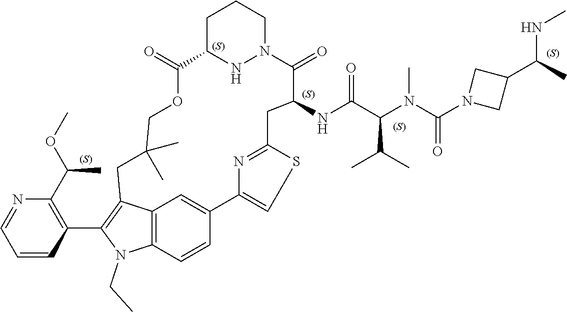 |
| A536 | 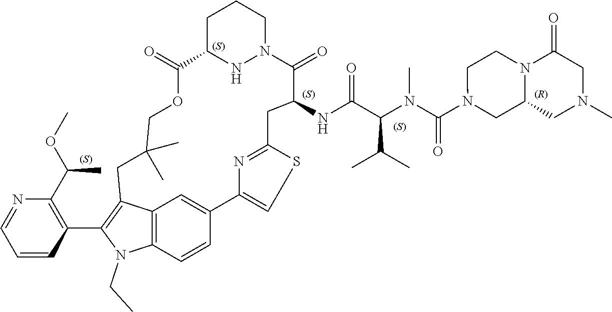 |
| A537 | 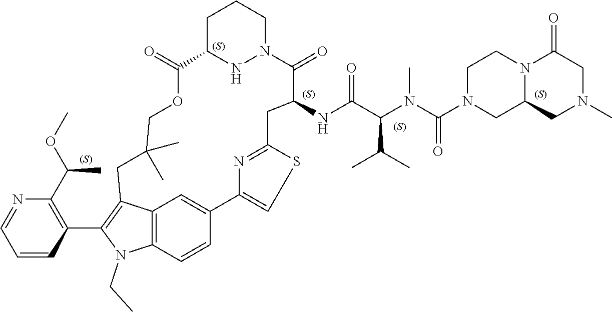 |
| A538 | 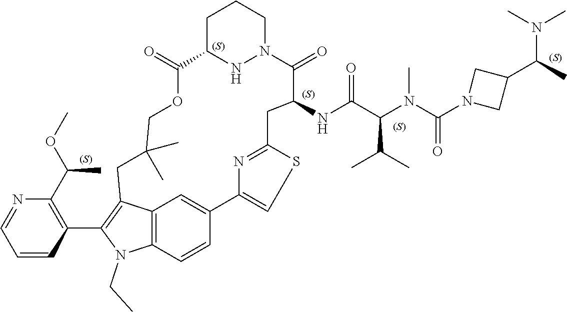 |
| A539 | 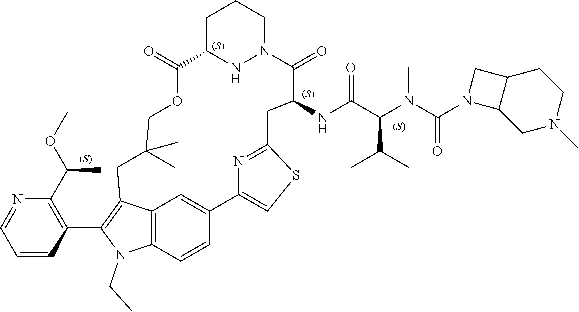 |
| A540 | 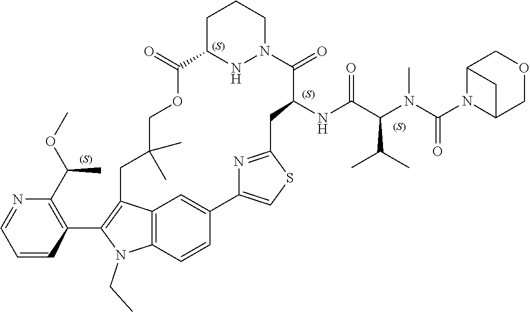 |
| A541 | 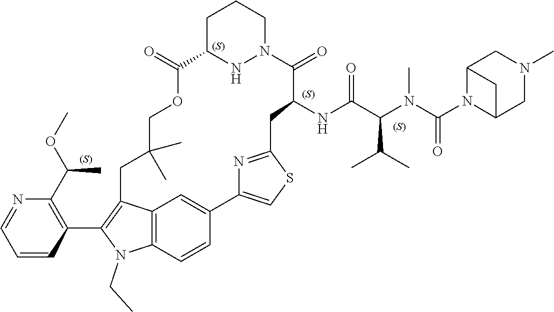 |
| A542 | 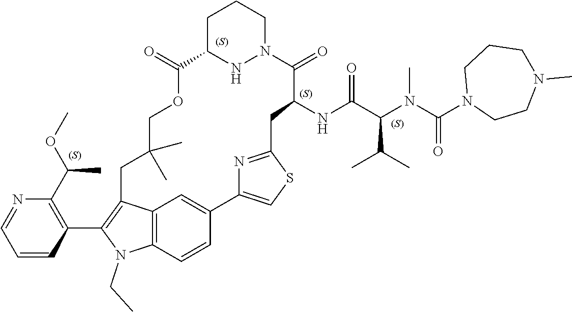 |
| A543 | 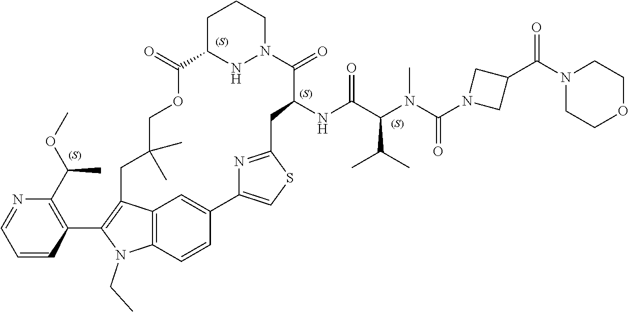 |
| A544 | 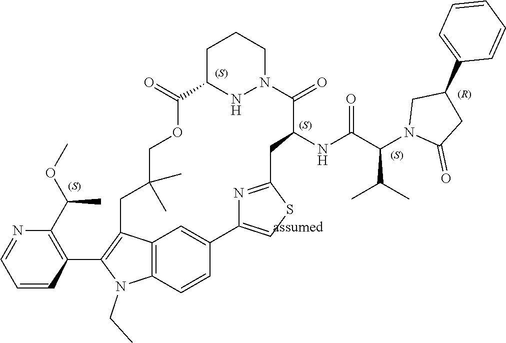 |
| A545 | 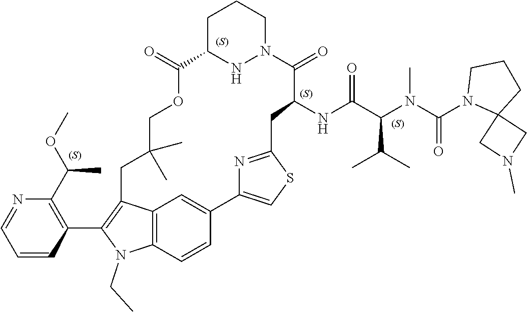 |
| A546 | 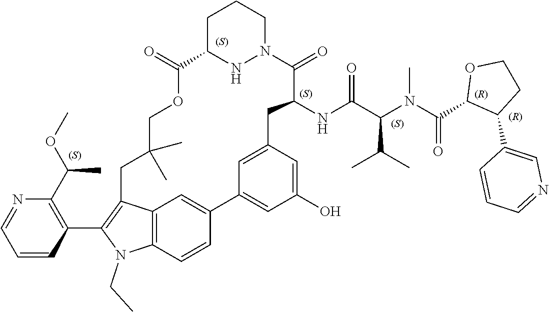 |
| A547 | 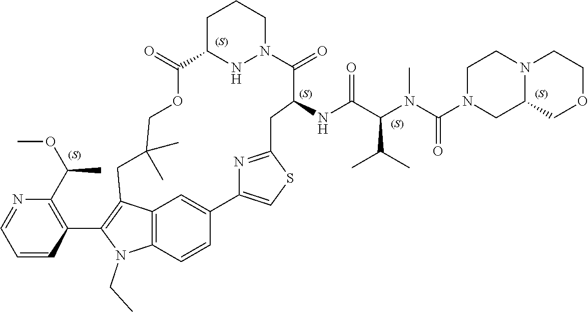 |
| A548 | 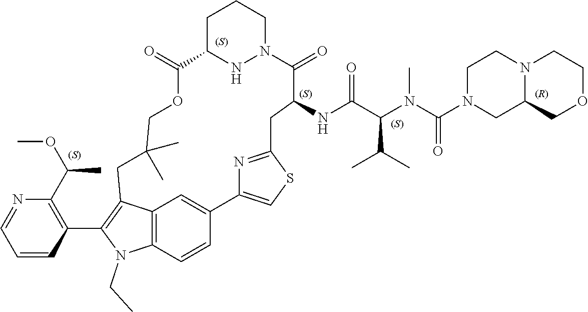 |
| A549 | 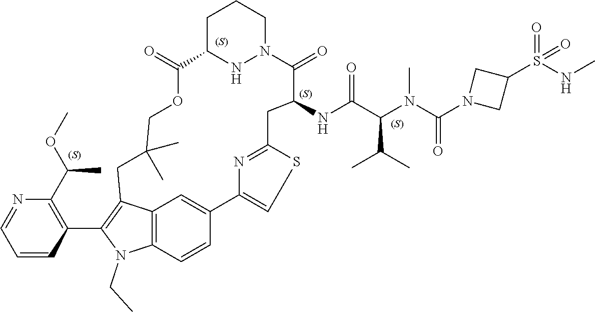 |
| A550 | 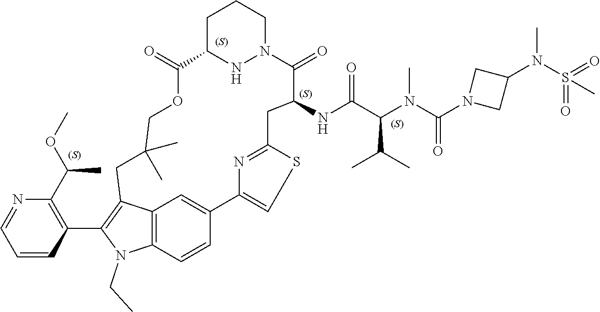 |
| A551 | 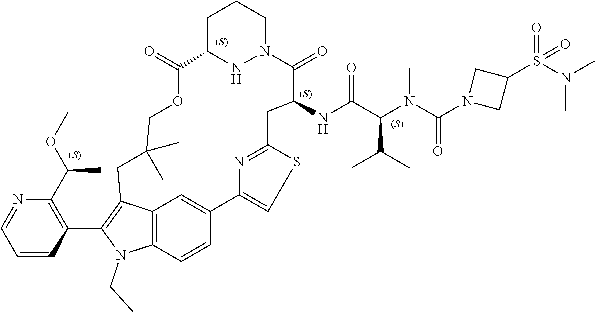 |
| A552 | 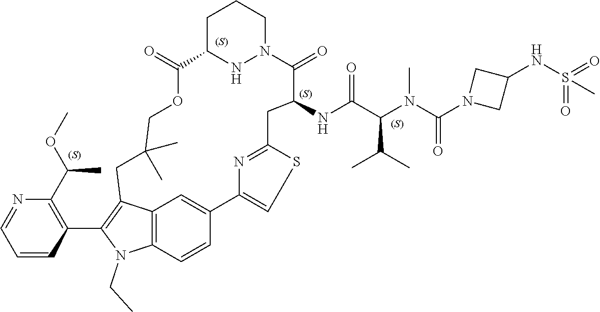 |
| A553 | 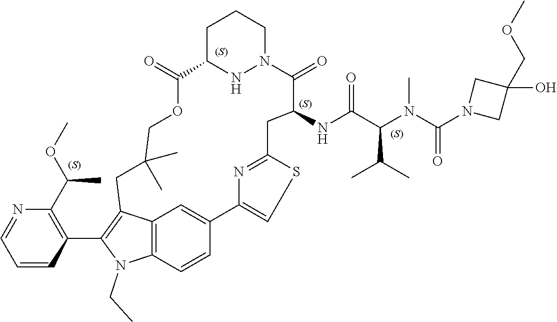 |
| A554 | 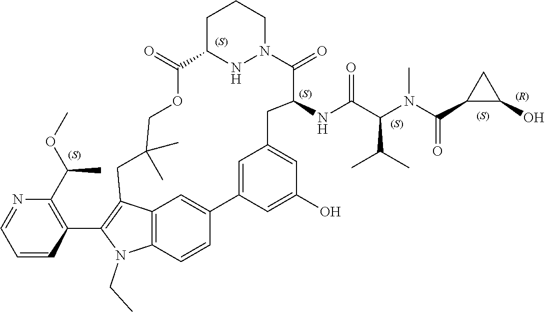 |
| A555 | 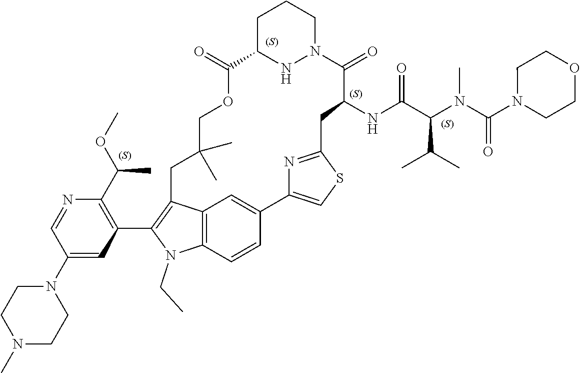 |
| A556 | 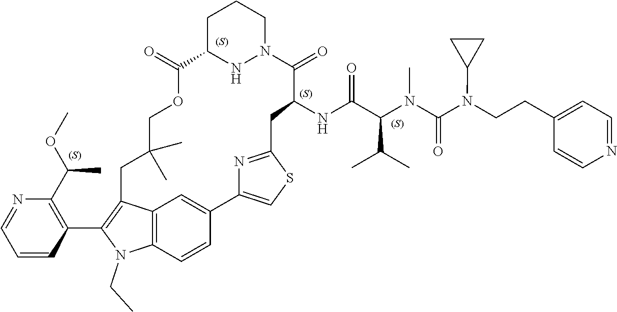 |
| A557 | 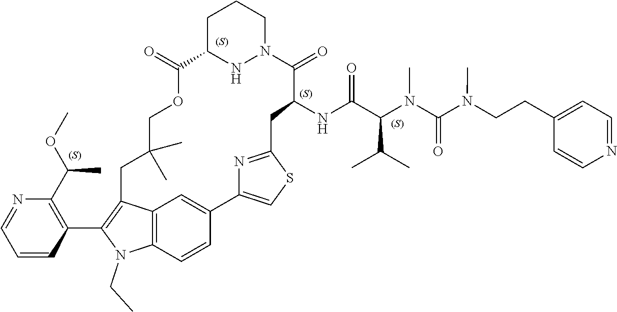 |
| A558 | 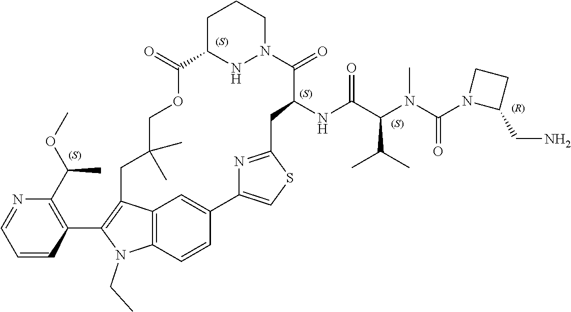 |
| A559 | 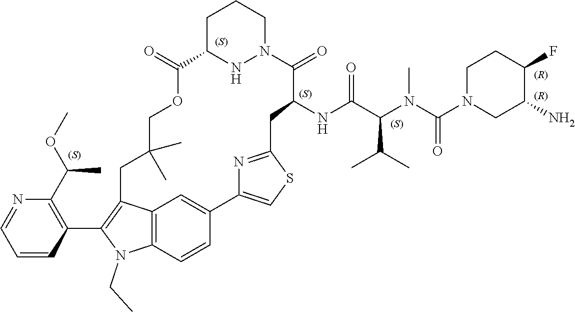 |
| A560 | 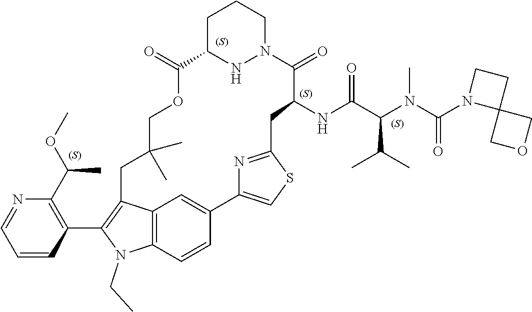 |
| A561 | 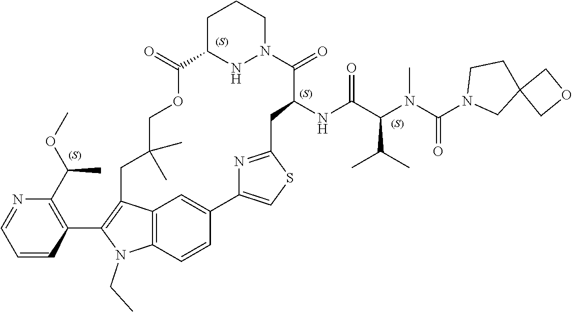 |
| A562 | 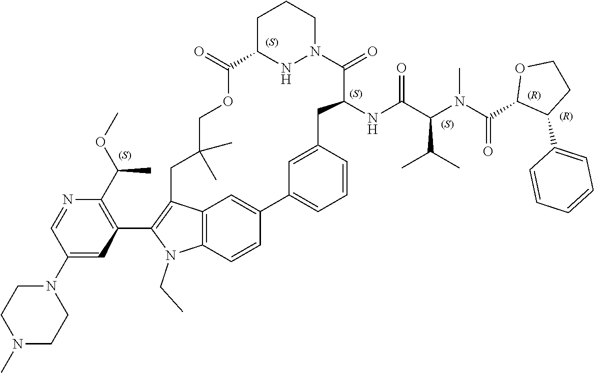 |
| A563 | 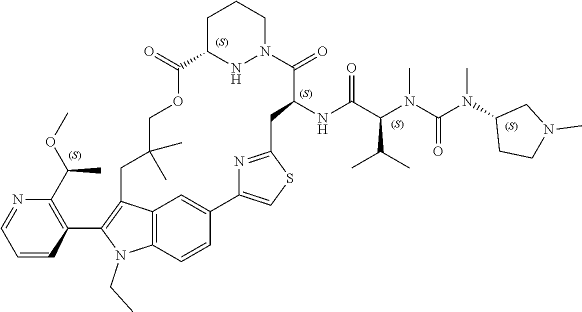 |
| A564 | 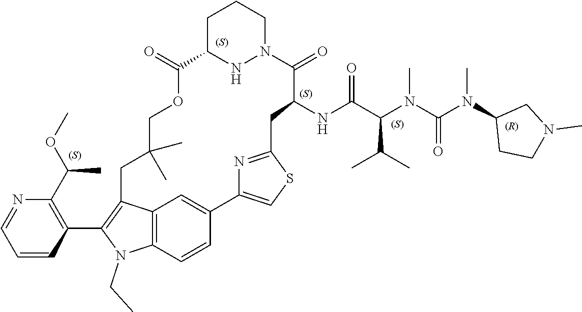 |
| A565 | 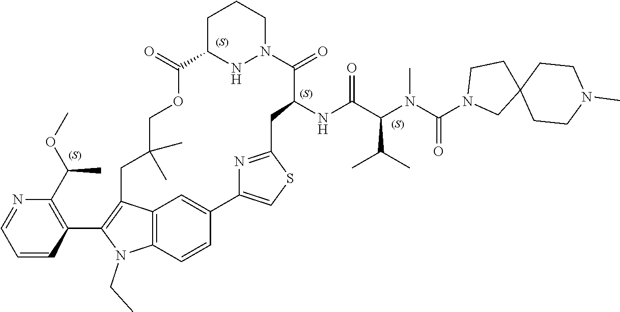 |
| A566 | 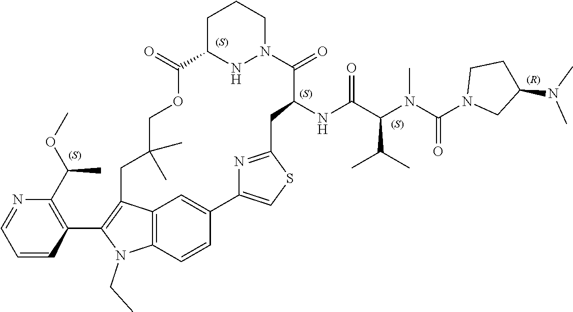 |
| A567 | 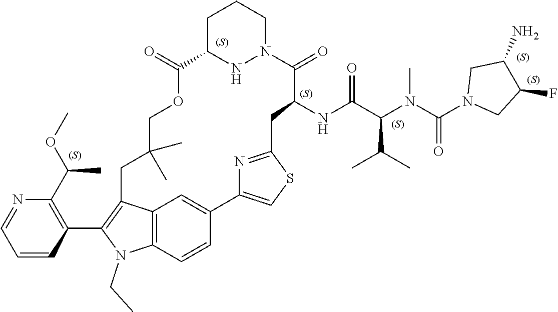 |
| A568 | 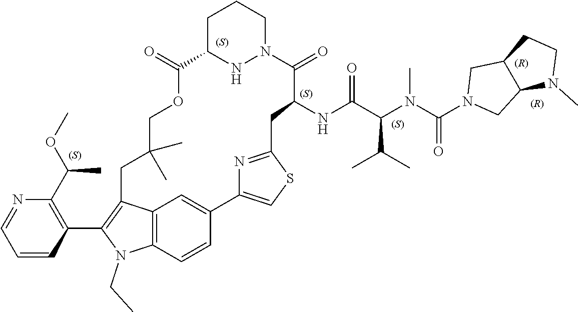 |
| A569 | 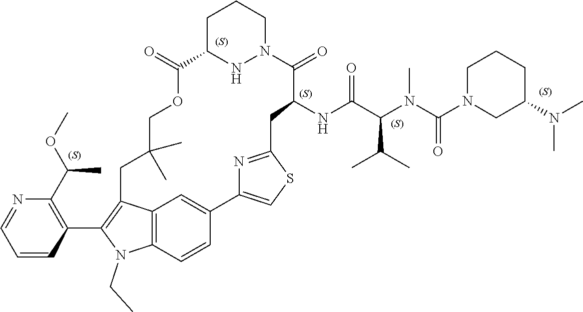 |
| A570 | 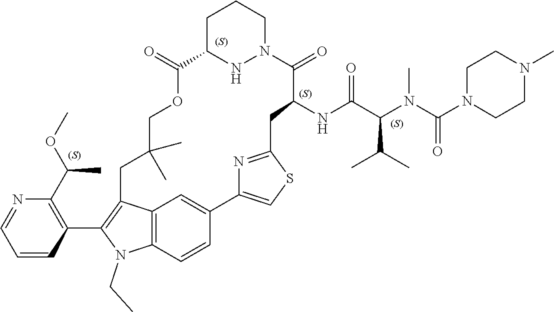 |
| A571 | 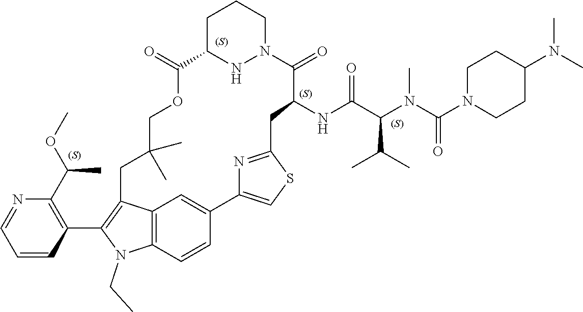 |
| A572 | 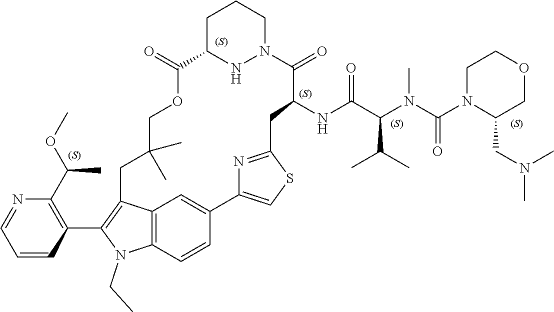 |
| A573 | 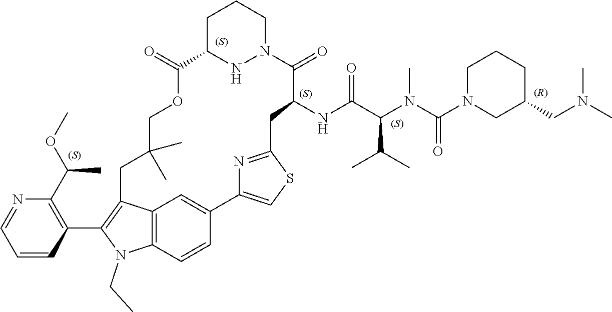 |
| A574 | 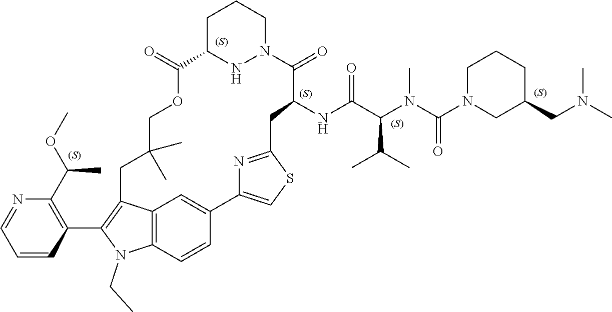 |
| A575 | 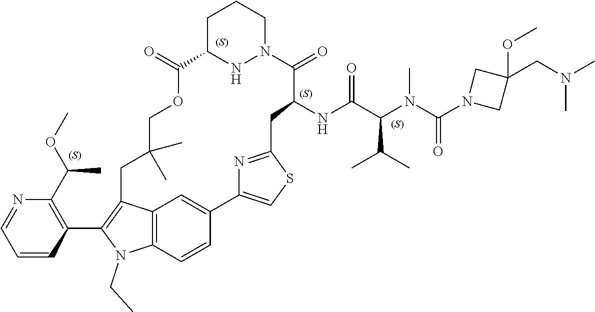 |
| A576 | 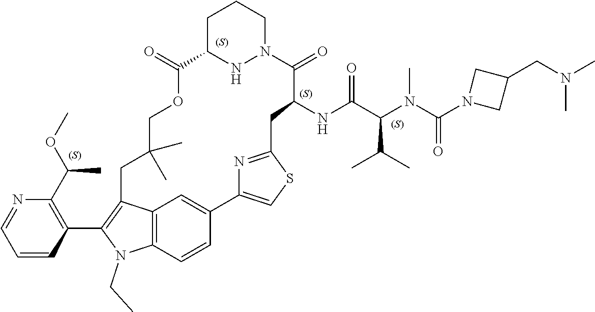 |
| A577 | 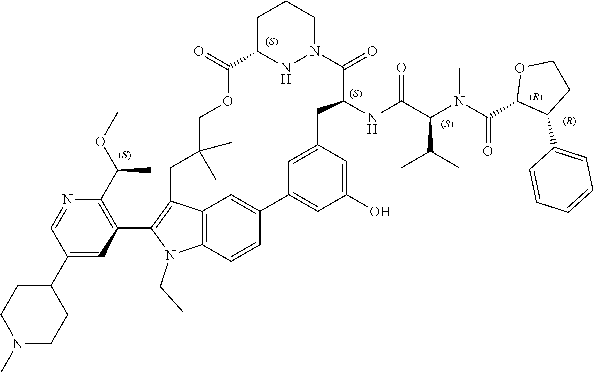 |
| A578 | 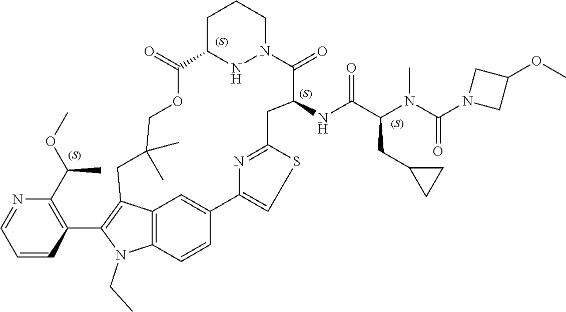 |
| A579 | 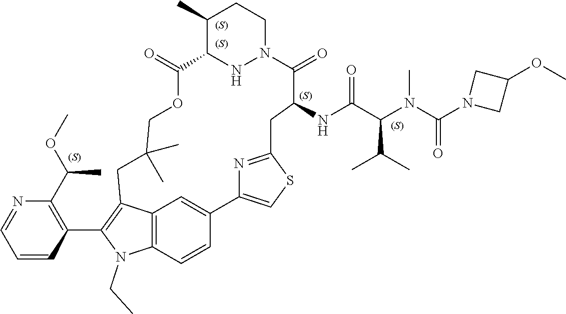 |
| A580 | 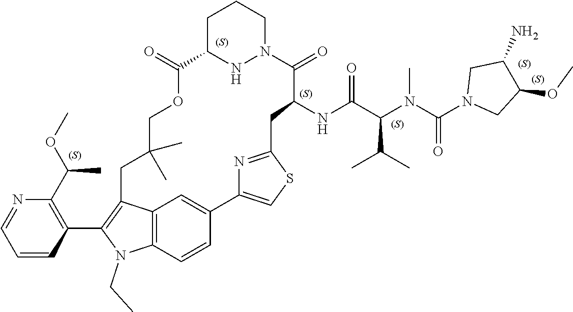 |
| A581 | 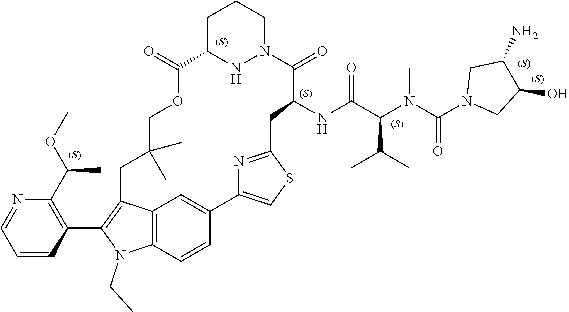 |
| A582 | 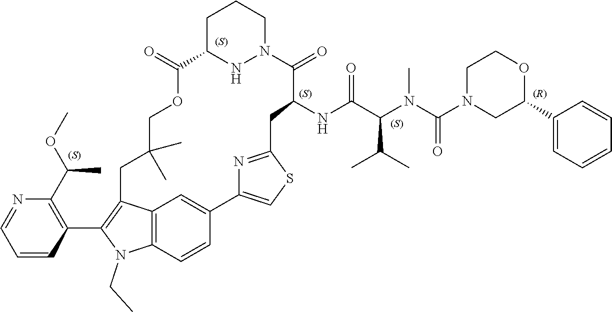 |
| A583 | 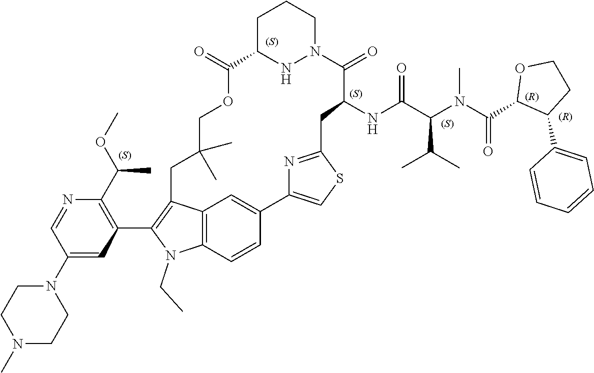 |
| A584 | 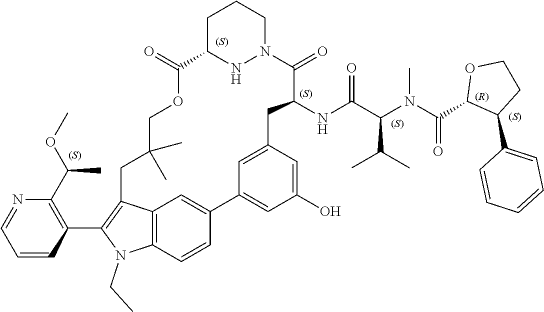 |
| A585 | 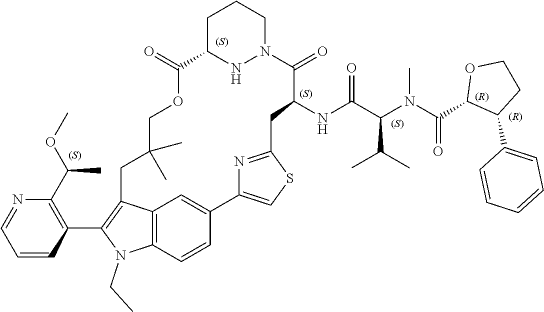 |
| A586 | 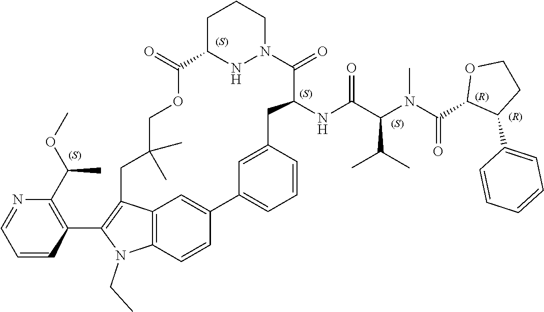 |
| A587 | 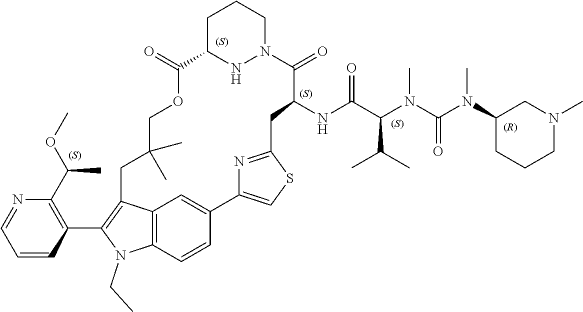 |
| A588 | 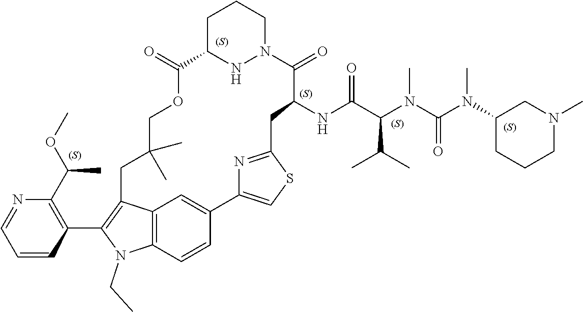 |
| A589 | 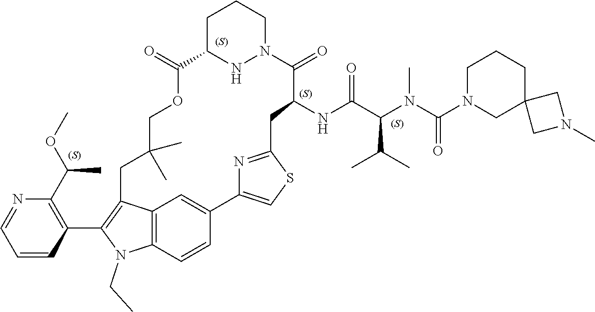 |
| A590 | 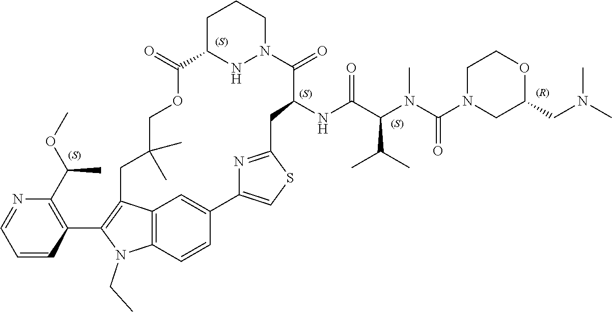 |
| A591 | 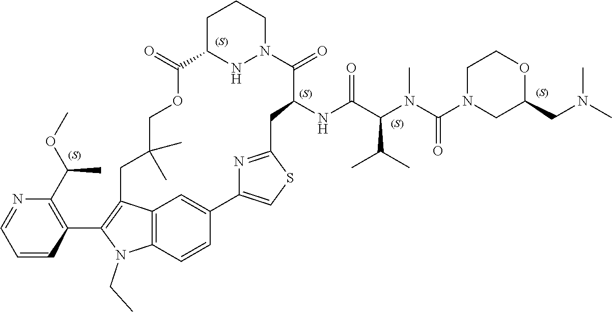 |
| A592 | 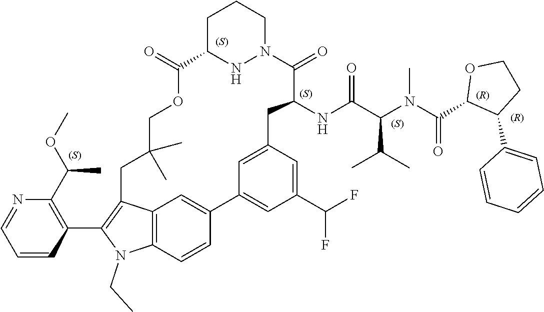 |
| A593 | 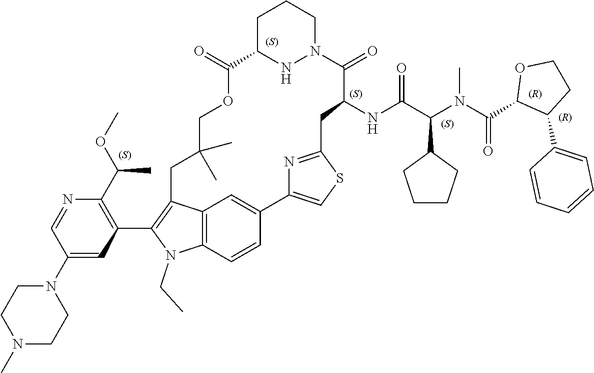 |
| A594 | 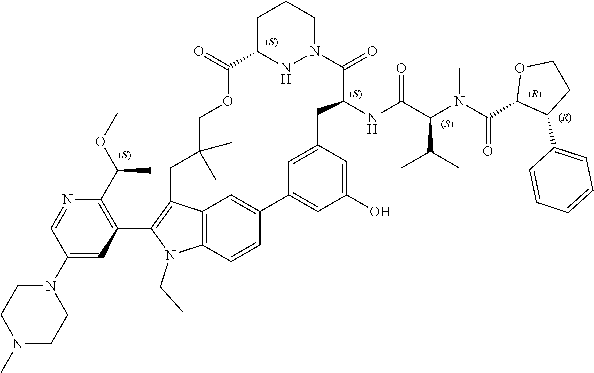 |
| A595 | 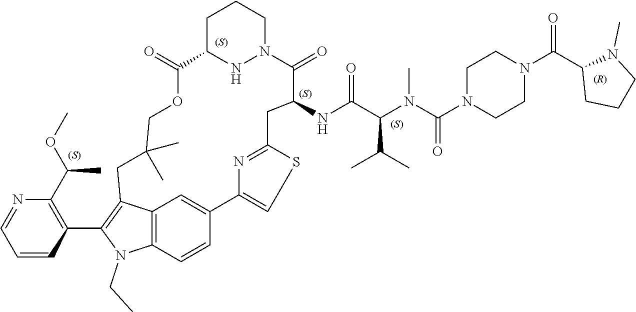 |
| A596 | 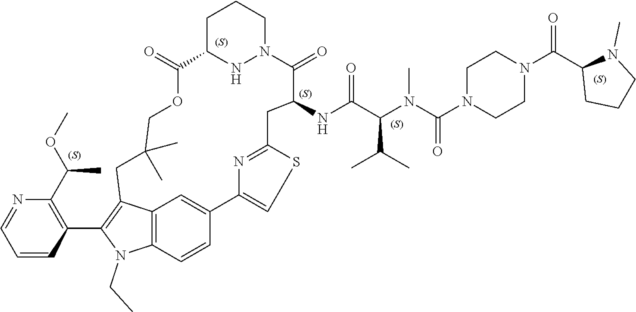 |
| A597 | 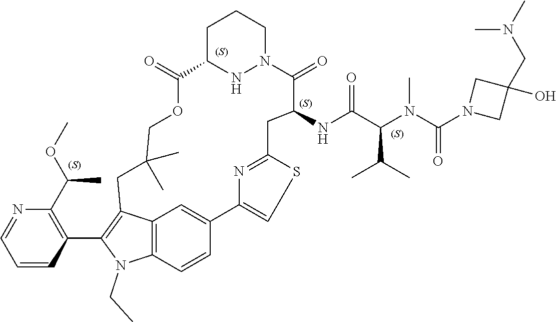 |
| A598 | 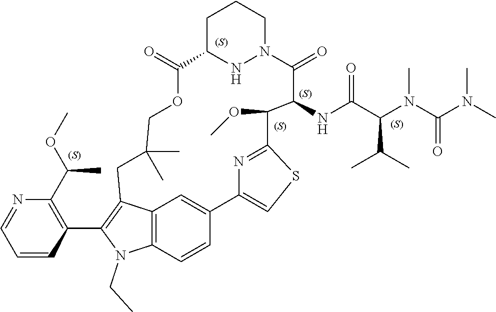 |
| A599 | 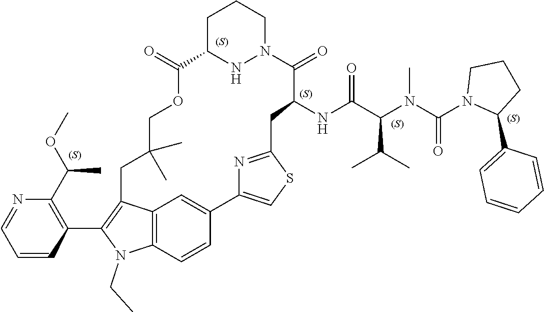 |
| A600 | 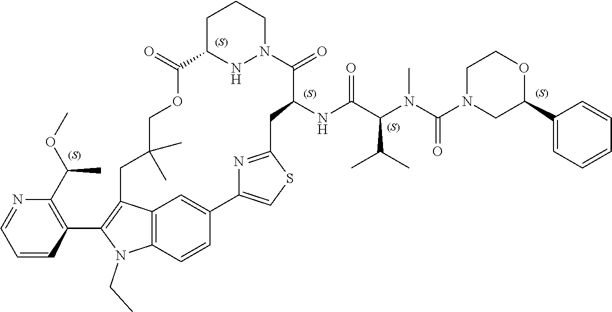 |
| A601 | 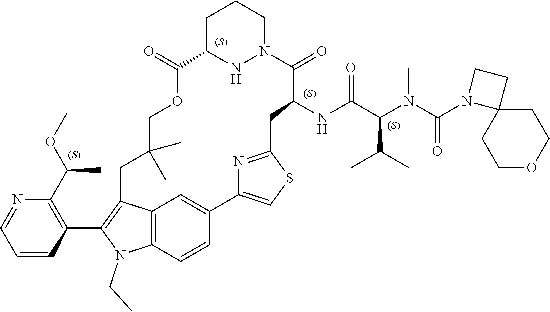 |
| A602 | 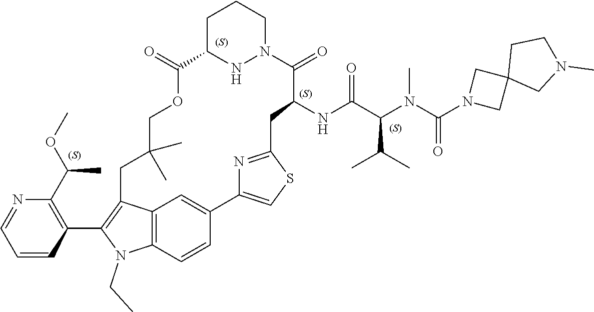 |
| A603 | 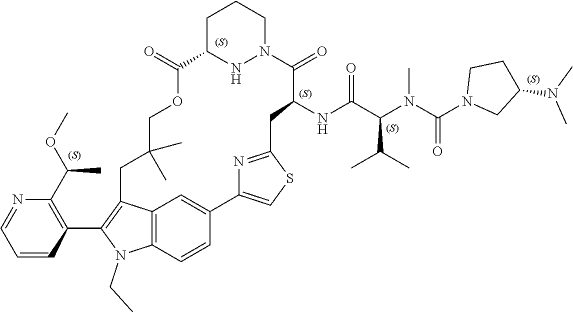 |
| A604 | 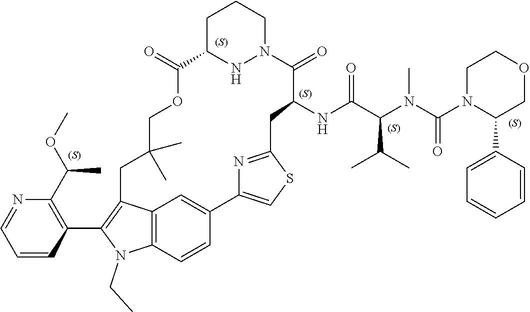 |
| A605 | 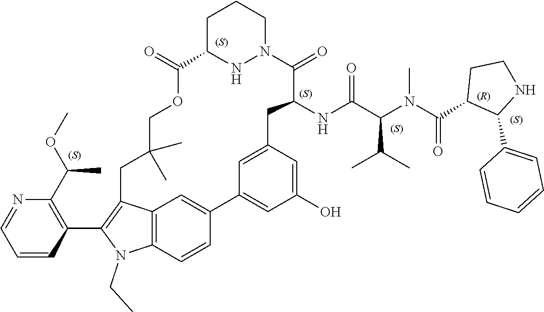 |
| A606 | 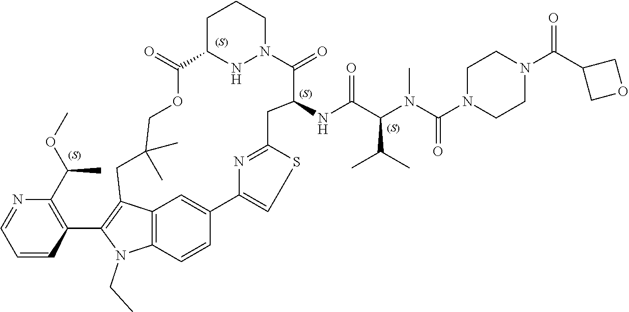 |
| A607 | 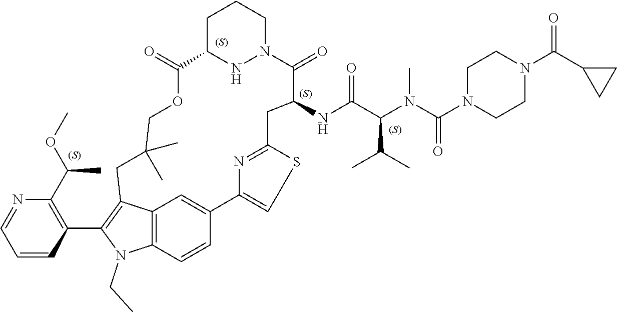 |
| A608 | 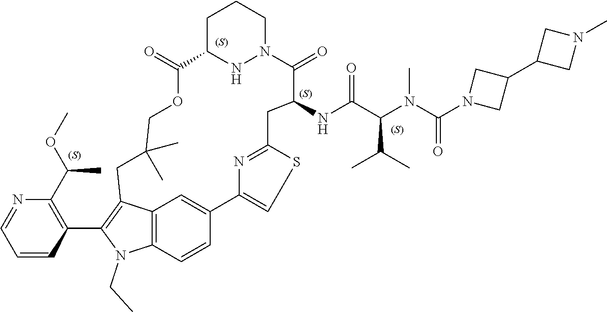 |
| A609 | 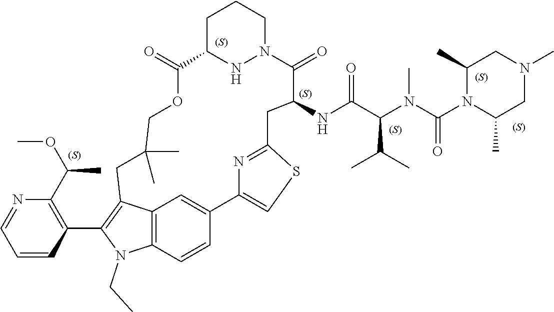 |
| A610 | 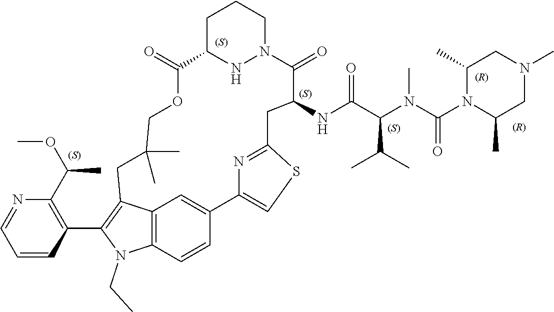 |
| A611 | 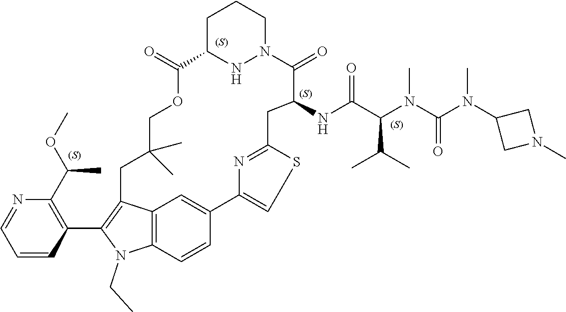 |
| A612 | 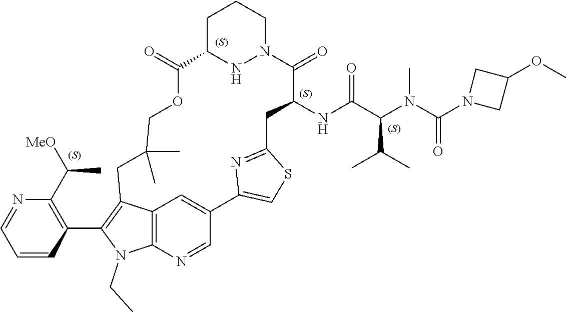 |
| A613 | 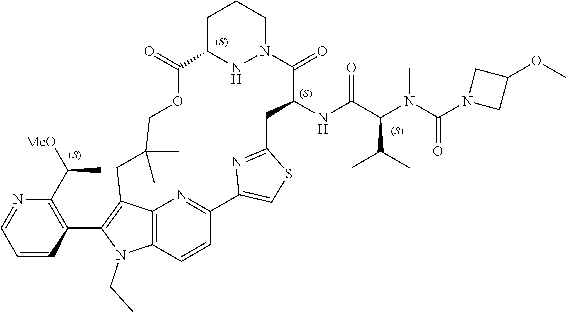 |
| A614 | 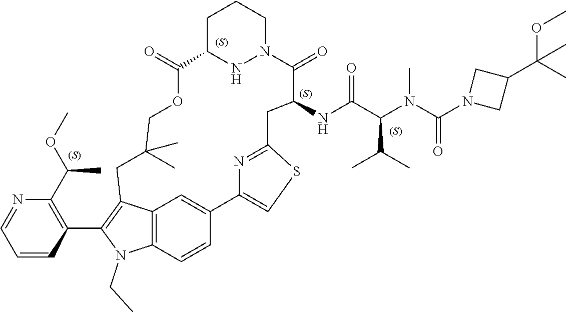 |
| A615 | 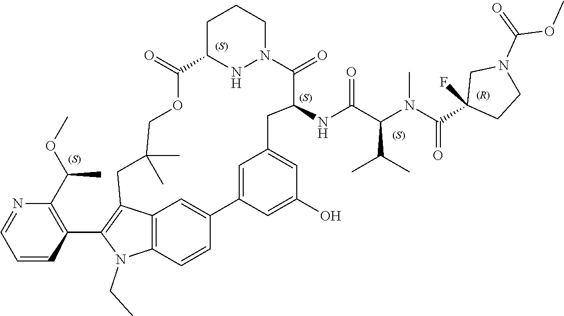 |
| A616 | 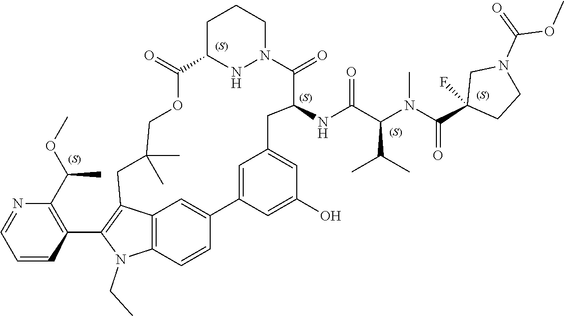 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated. Any compound shown in brackets indicates that the compound is a disastereomer, and the absolute stereochemistry of such diastereomer may not be known.

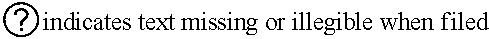

In some embodiments, a compound of Table 2 is provided, or a pharmaceutically acceptable salt thereof. In some embodiments, a compound of the present invention is selected from Table 2, or a pharmaceutically acceptable salt or atropisomer thereof.

TABLE 2
Certain Compounds of the Present Invention

| Ex# | Structure |
|---|---|
| B4 | 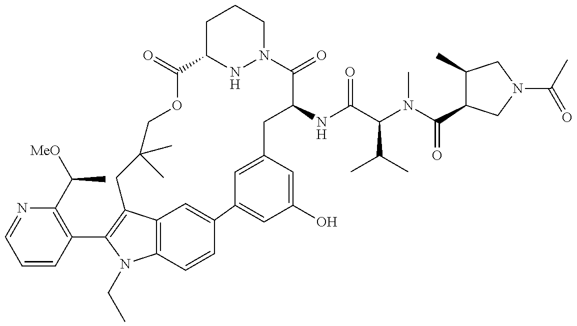 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| B5 | 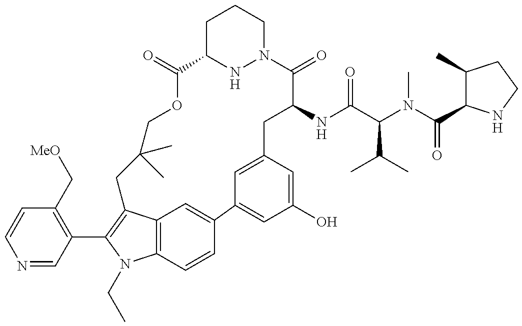 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| B6 | 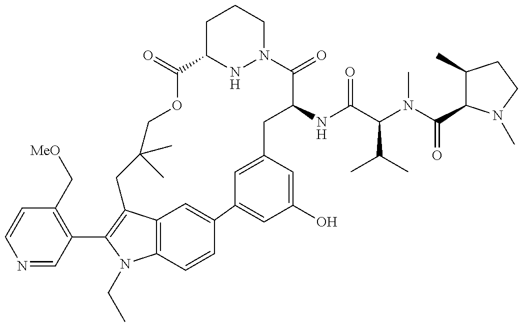 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| B8 | 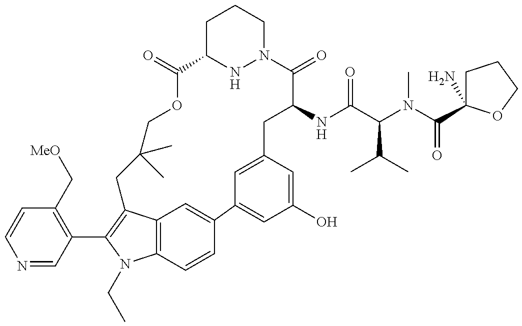 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCCO1)C(=O)N1CCC[C@H](N1)C(=O)OCC(C)(C)C2] |
| B9 | 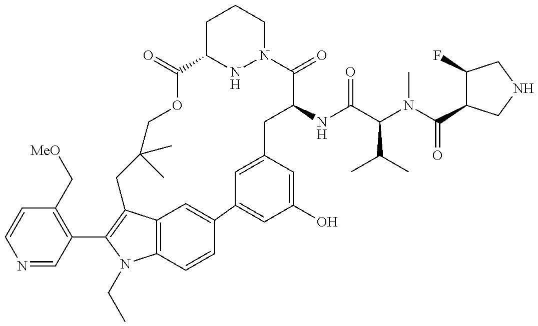 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| B12 | 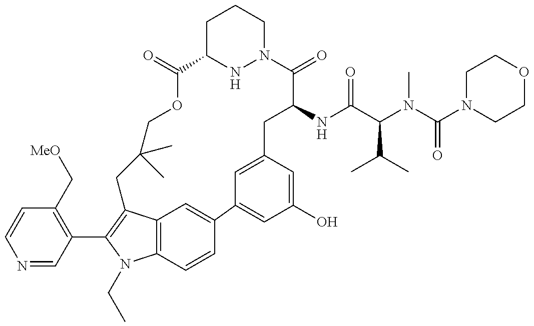 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| B13 | 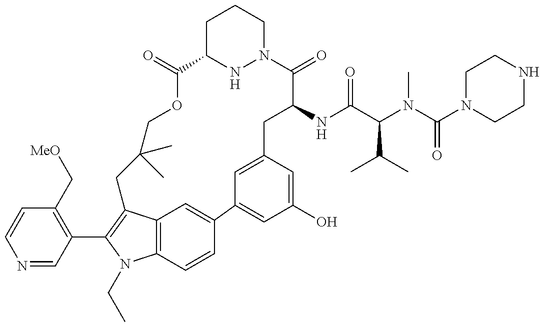 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| B19 | 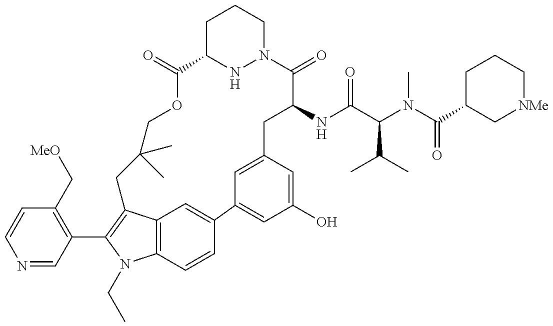 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| B44 | 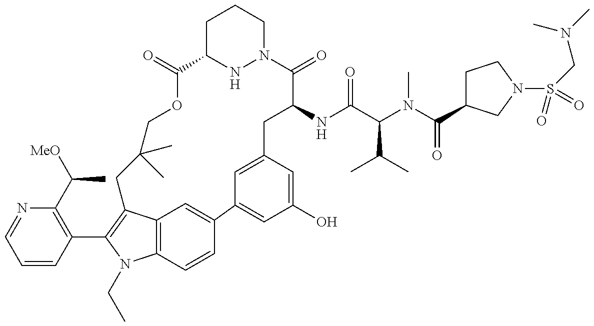 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)CN(C)C)C1)C(=O)N1CCC[C@H](N1)C(=O)OCC(C)(C)C2] |
| B47 | 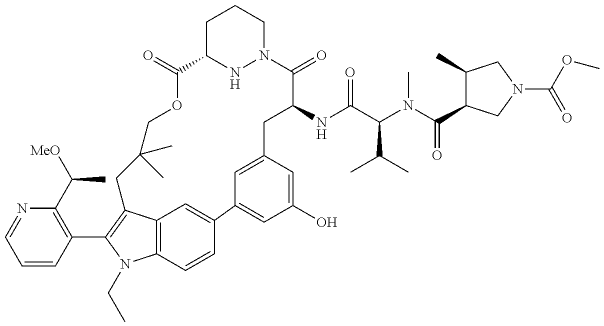 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2] |
| B57 | 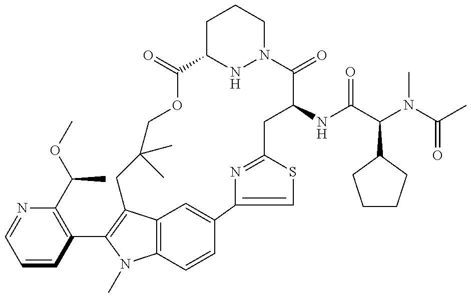 |
| B58 | 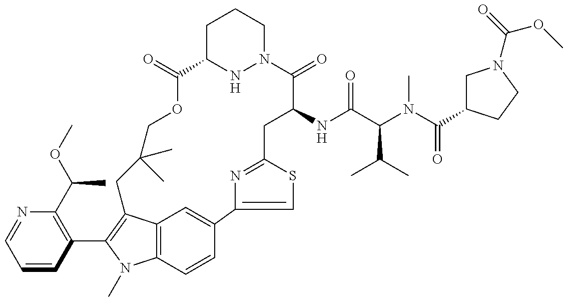 |
| B59 | 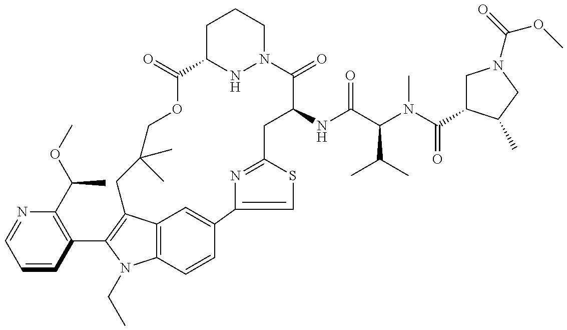 |
| B60 | 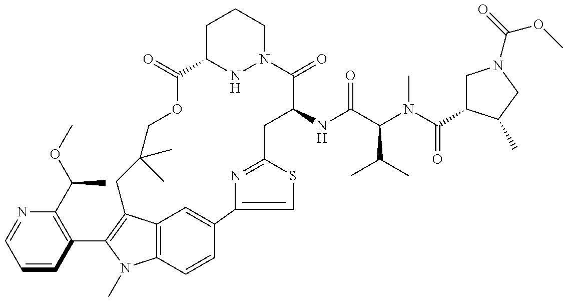 |
| B61 | 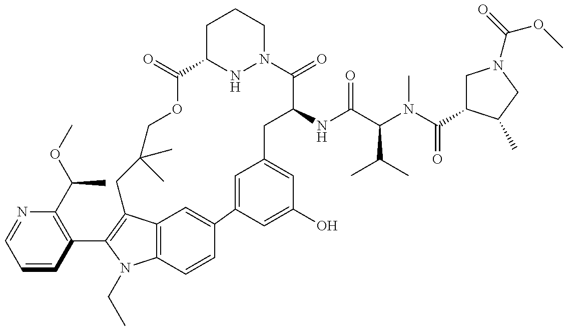 |
| B66 | 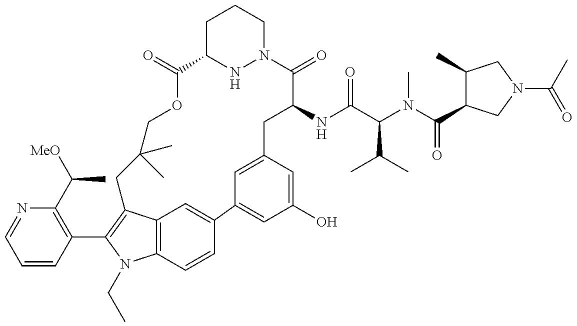 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| B67 | 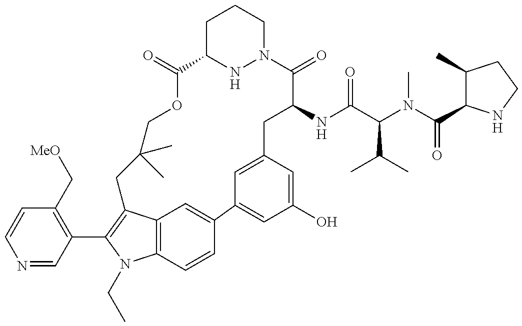 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| B69 | 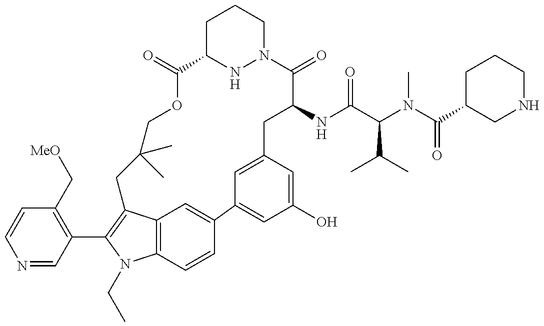 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCNC1)C(=O)N1CCC[C@H](N1)C(=O)OCC(C)(C)C2] |
| B71 | 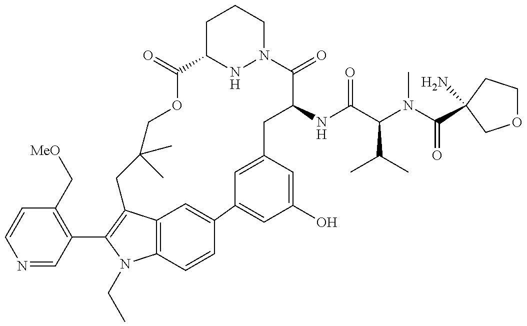 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| B73 | 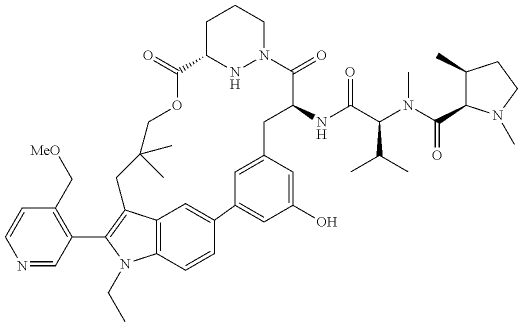 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| B74 | 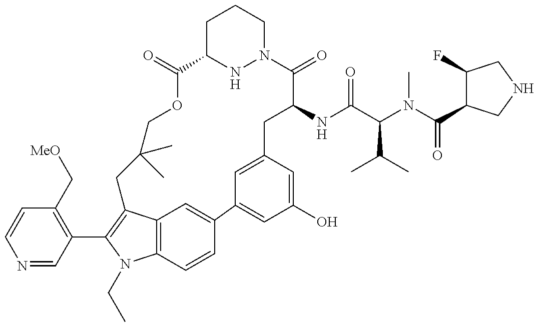 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| B80 | 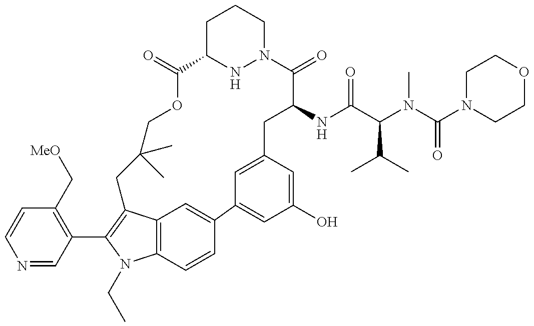 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| B81 | 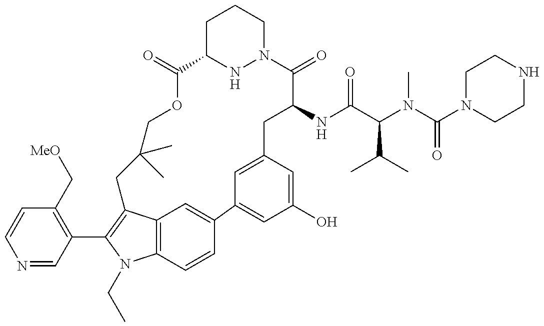 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| B94 | 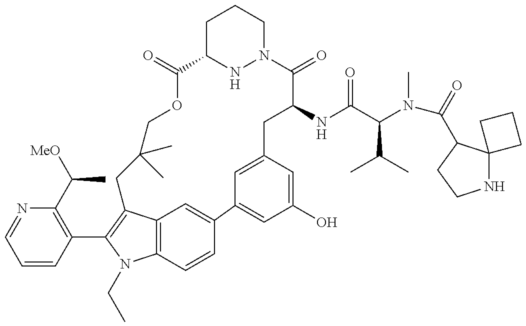 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CCC3)C(=O)N1CCC[C@H](N1)C(=O)OCC(C)(C)C2] |
| B95 | 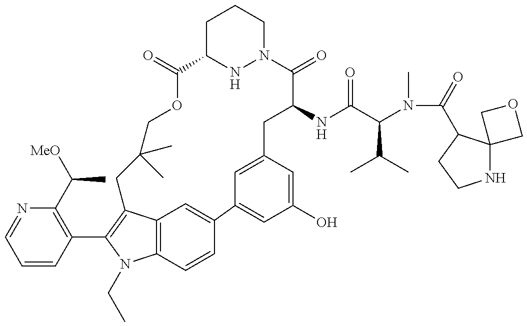 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13COC3)C(=O)N1CCC[C@H](N1)C(=O)OCC(C)(C)C2] |
| B96 | 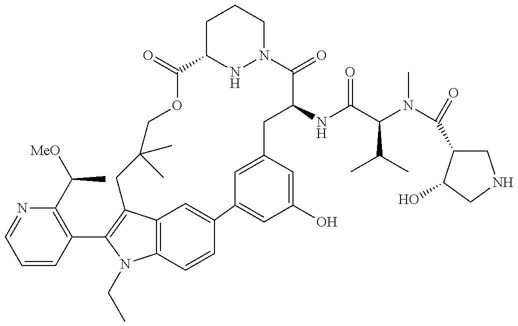 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| B97 | 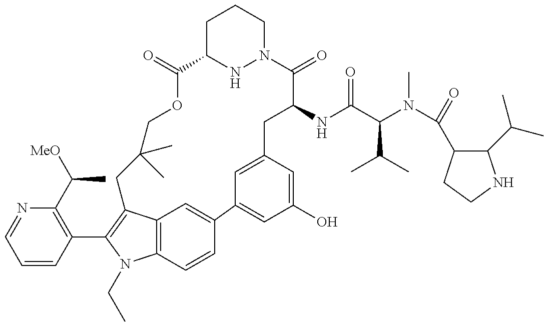 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC1C(C)C)C(=O)N1CCC[C@H](N1)C(=O)OCC(C)(C)C2] |
| B99 | 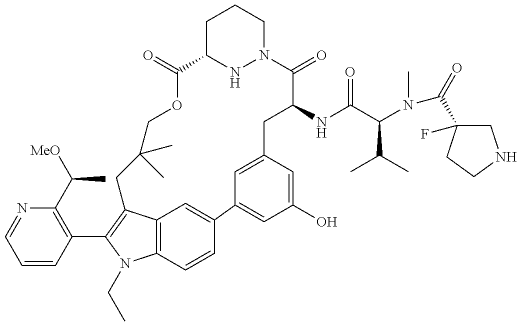 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(F)CCNC1)C(=O)N1CCC[C@H](N1)C(=O)OCC(C)(C)C2] |
| B100 | 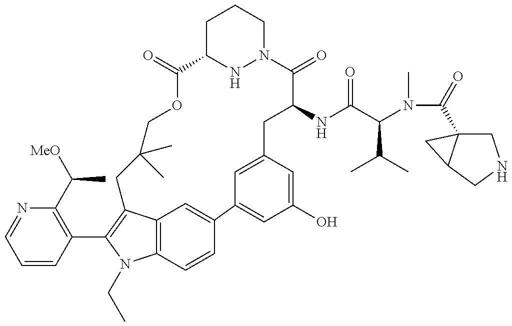 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]13CNCC1C3)C(=O)N1CCC[C@H](N1)C(=O)OCC(C)(C)C2] |
| B104 | 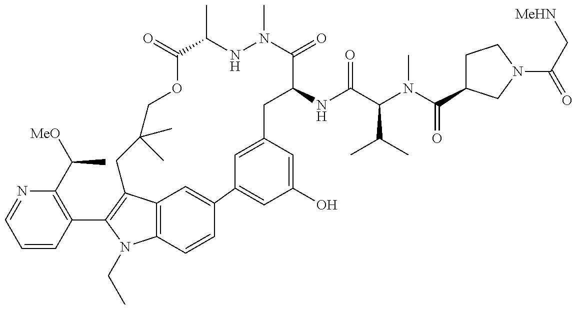 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N(C)N[C@@H](C)C(=O)OCC(C)(C)C2] |
| B106 | 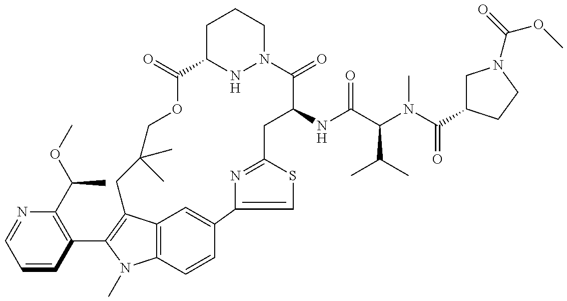 |
| B107 | 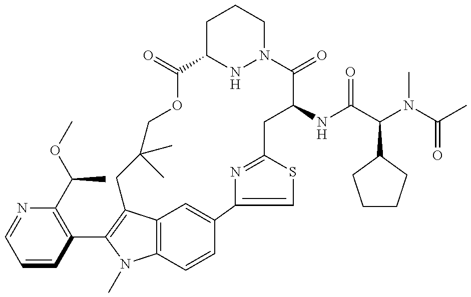 |
| B109 | 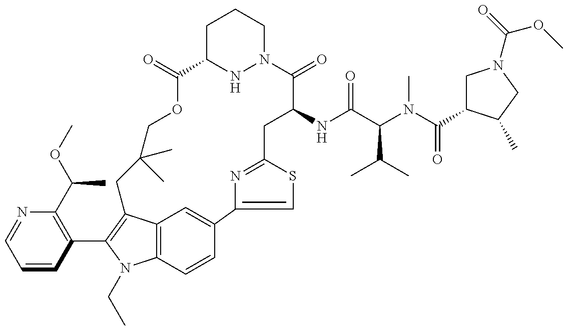 |
| B110 | 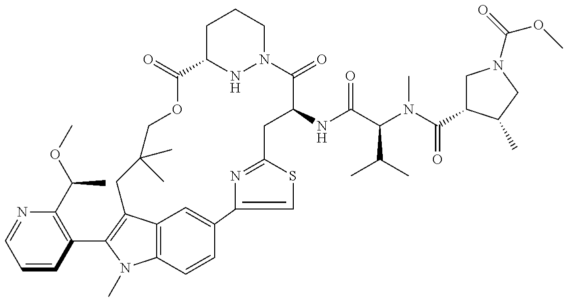 |
| B111 | 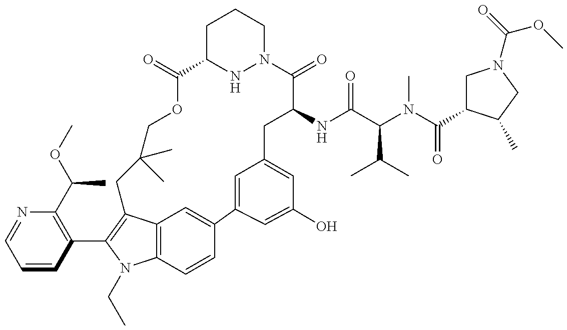 |
| B112 | 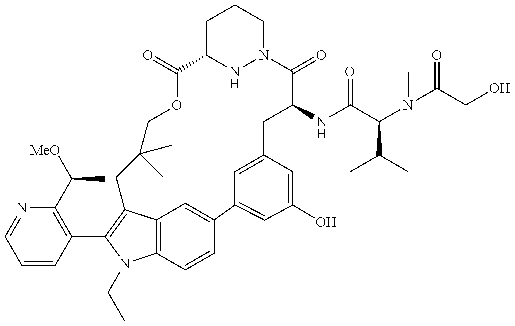 [SMILES: CCn1c(-c2cccnc2[C@H](C)OC)c2c3cc(ccc31)-c1cc(O)cc(c1)C[C@H](NC(=O)[C@H](C(C)C)N(C)C(=O)CO)C(=O)N1CCC[C@H](N1)C(=O)OCC(C)(C)C2] |
| B113 | 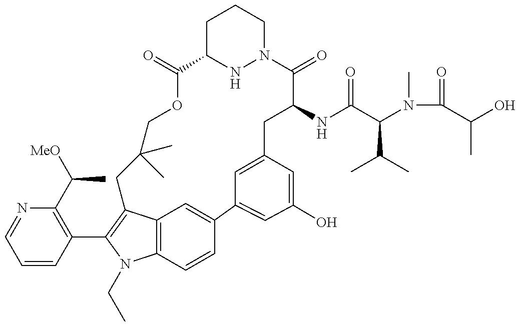 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(C)O)C(=O)N1CCC[C@H](N1)C(=O)OCC(C)(C)C2] |
| B114 | 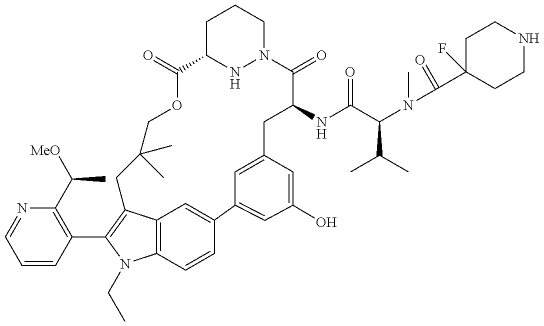 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(F)CCNCC1)C(=O)N1CCC[C@H](N1)C(=O)OCC(C)(C)C2] |
| B117 | 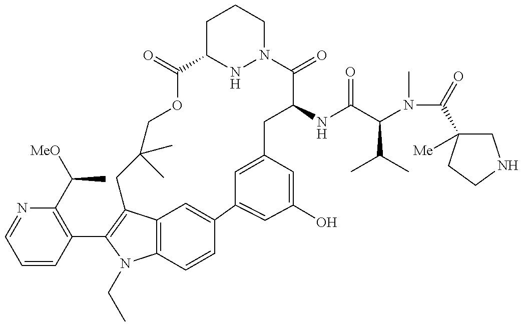 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCNC1)C(=O)N1CCC[C@H](N1)C(=O)OCC(C)(C)C2] |
| B119 | 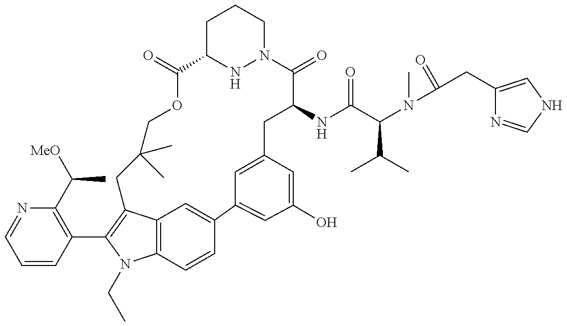 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1c[nH]cn1)C(=O)N1CCC[C@H](N1)C(=O)OCC(C)(C)C2] |
| B122 | 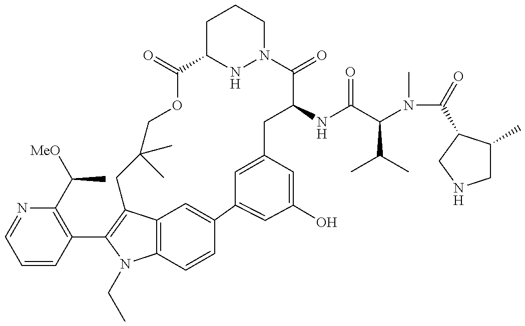 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CNC[C@H]1C)C(=O)N1CCC[C@H](N1)C(=O)OCC(C)(C)C2] |
| B123 | 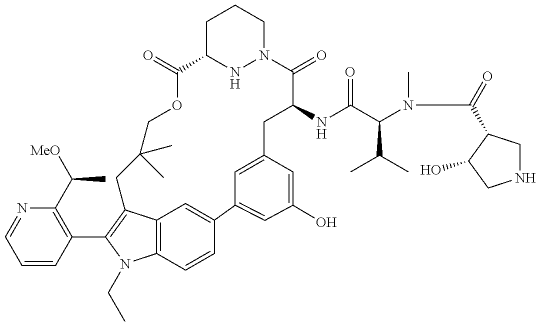 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| B124 | 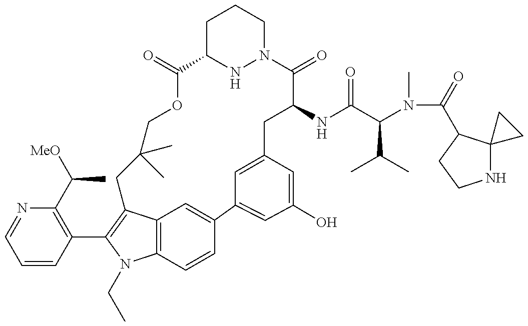 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CC3)C(=O)N1CCC[C@H](N1)C(=O)OCC(C)(C)C2] |
| B126 | 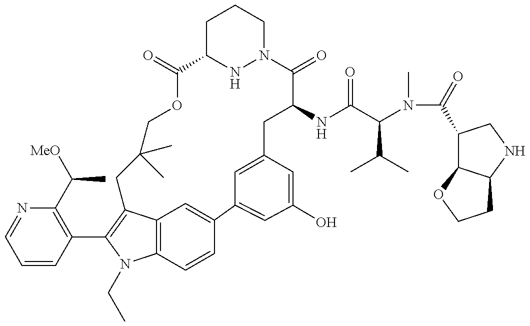 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN[C@H]3CCO[C@H]31)C(=O)N1CCC[C@H](N1)C(=O)OCC(C)(C)C2] |
| B128 | 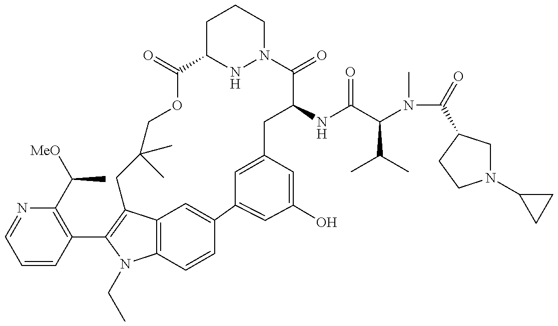 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C3CC3)C1)C(=O)N1CCC[C@H](N1)C(=O)OCC(C)(C)C2] |
| B129 | 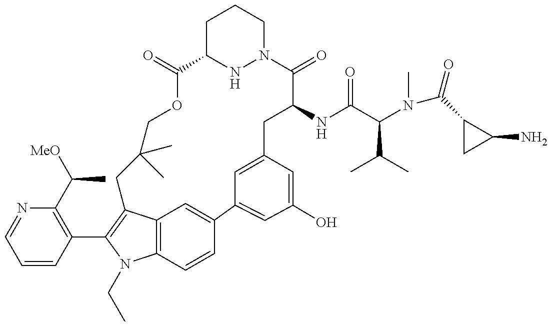 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H]1N)C(=O)N1CCC[C@H](N1)C(=O)OCC(C)(C)C2] |
| B130 | 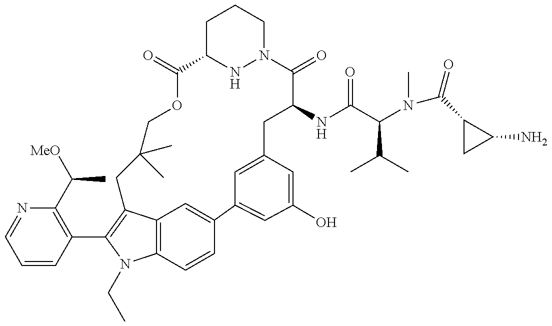 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| B133 | 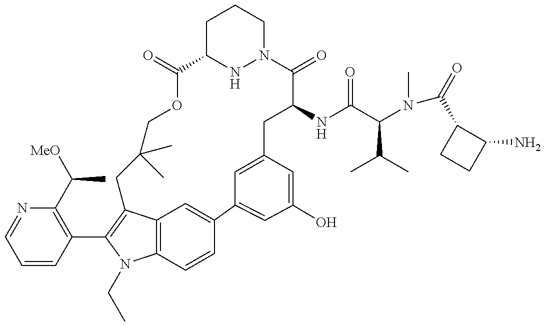 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CC[C@H]1N)C(=O)N1CCC[C@H](N1)C(=O)OCC(C)(C)C2] |
| B134 | 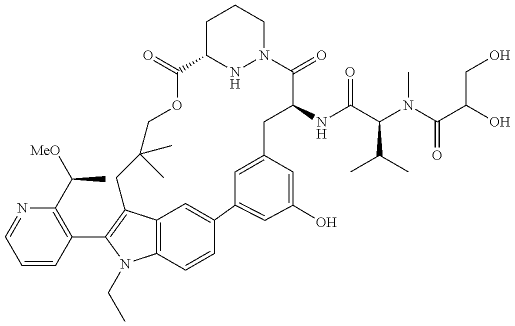 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(O)CO)C(=O)N1CCC[C@H](N1)C(=O)OCC(C)(C)C2] |
| B135 | 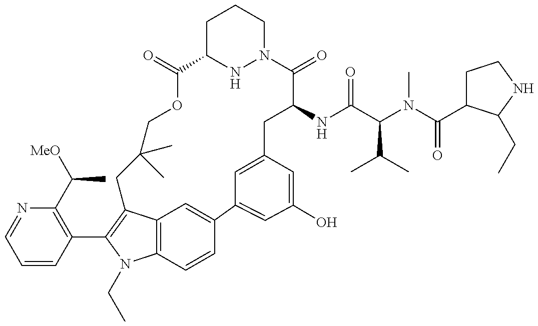 [SMILES: CCC1NCCC1C(=O)N(C)[C@H](C(=O)N[C@H]1Cc2cc(O)cc(c2)-c2ccc3c(c2)c(c(-c2cccnc2[C@H](C)OC)n3CC)CC(C)(C)COC(=O)[C@@H]2CCCN(N2)C1=O)C(C)C] |
| B137 | 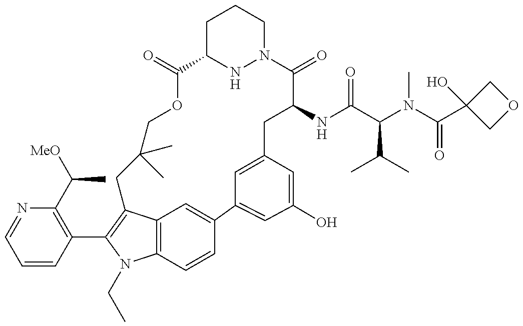 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| B138 | 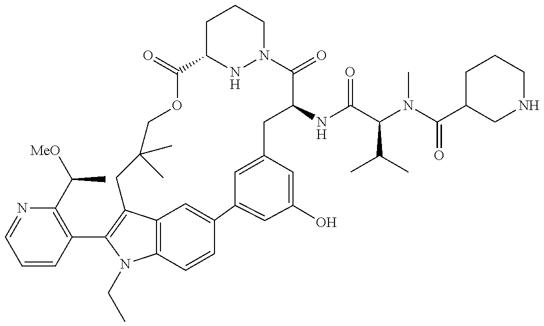 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCCNC1)C(=O)N1CCC[C@H](N1)C(=O)OCC(C)(C)C2] |
| B139 | 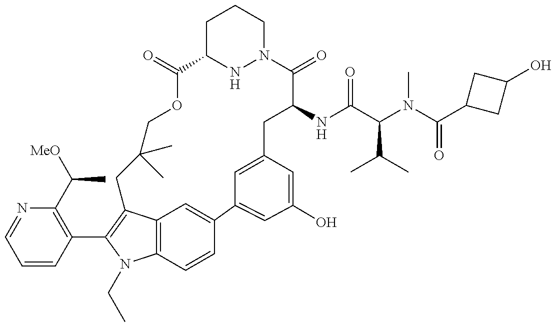 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC(O)C1)C(=O)N1CCC[C@H](N1)C(=O)OCC(C)(C)C2] |
| B141 | 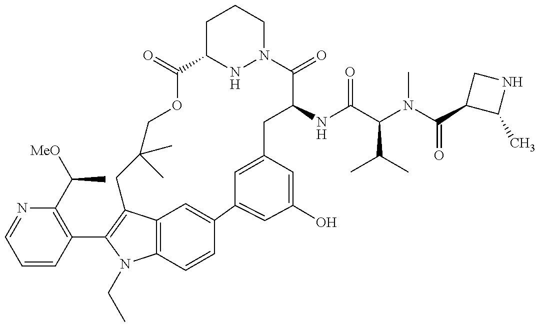 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CN[C@@H]1C)C(=O)N1CCC[C@H](N1)C(=O)OCC(C)(C)C2] |
| B143 | 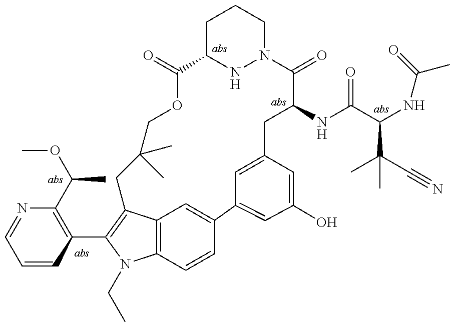 |
| B144 | 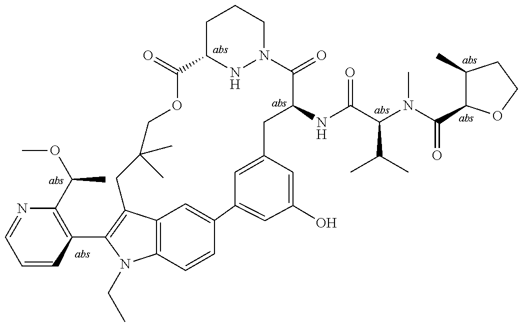 |
| B145 | 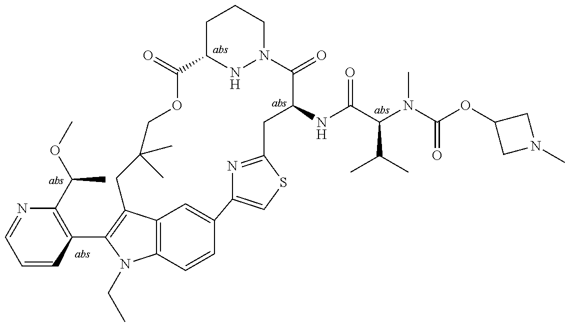 |
| B146 | 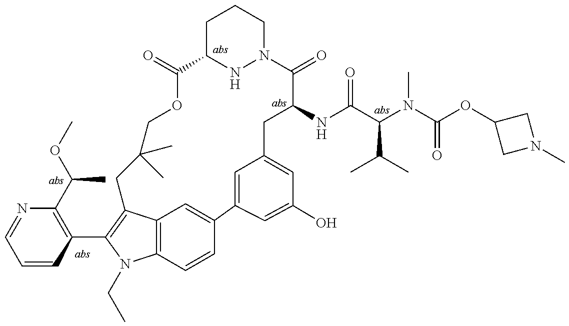 |
| B147 | 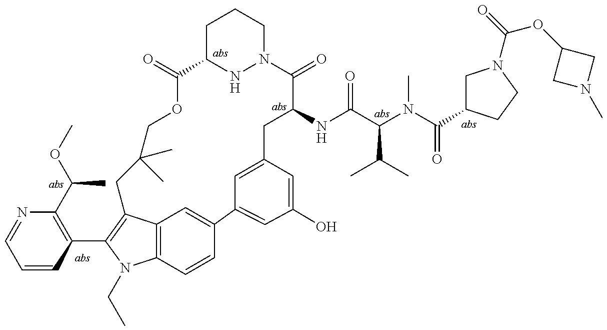 |
| B148 | 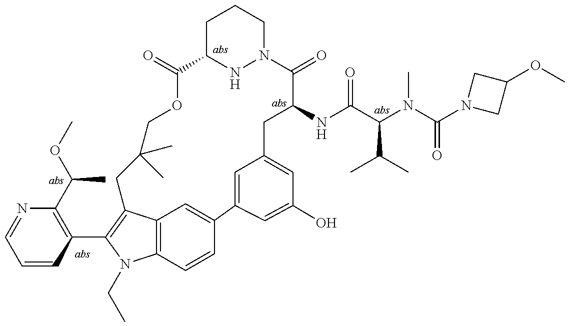 |
| B149 | 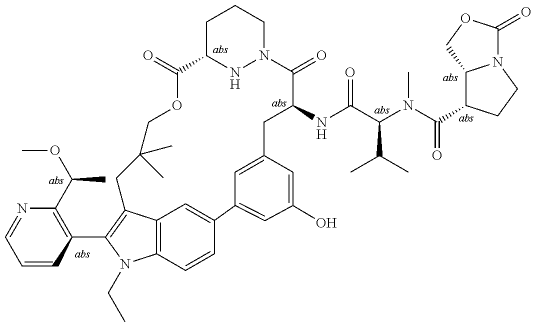 |
| B150 | 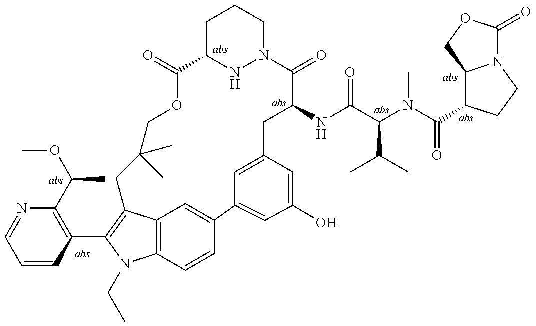 |
| B151 | 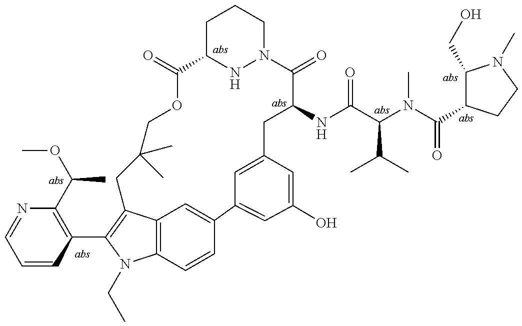 |
| B152 | 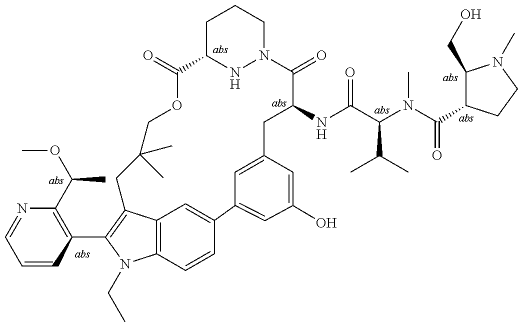 |
| B153 | 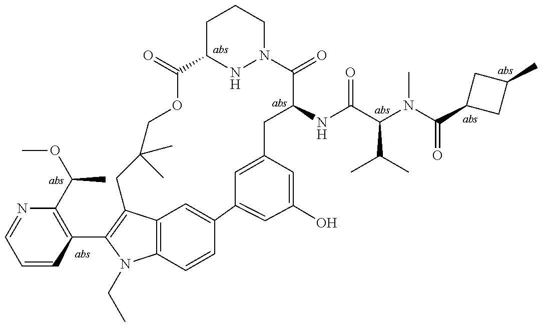 |
| B154 | 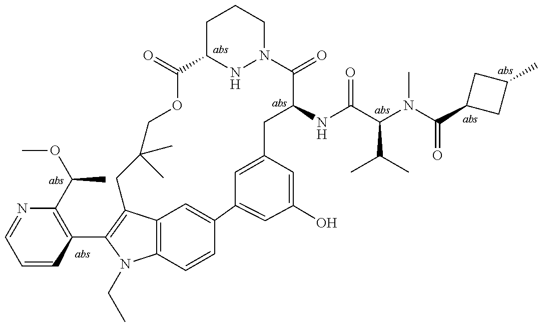 |
| B155 | 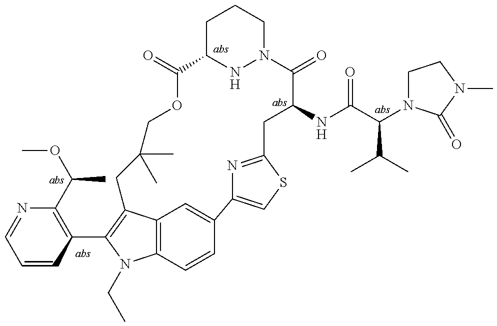 |
| B156 | 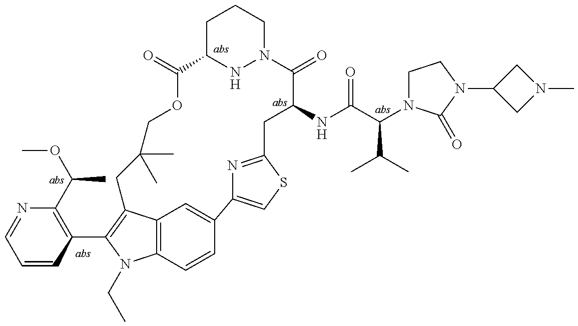 |
| B157 | 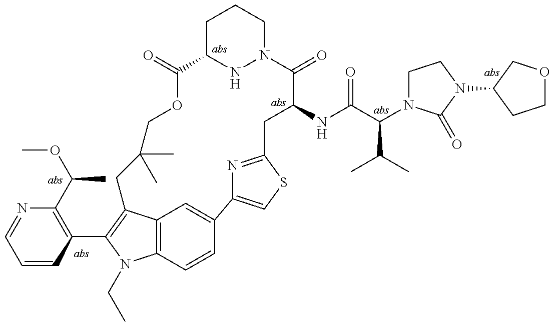 |
| B158 | 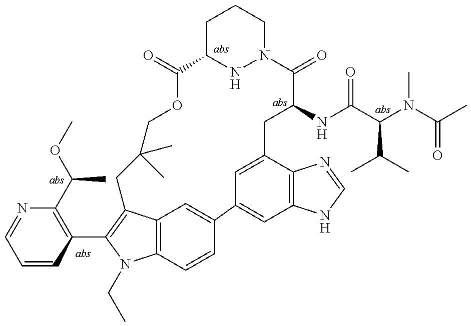 |
| B159 | 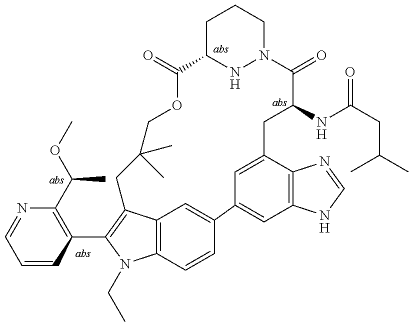 |
| B160 | 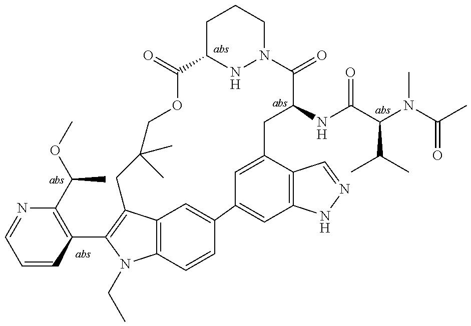 |
| B161 | 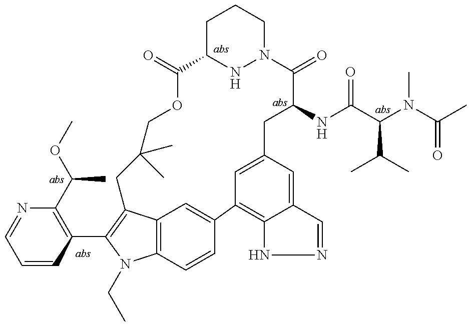 |
| B162 | 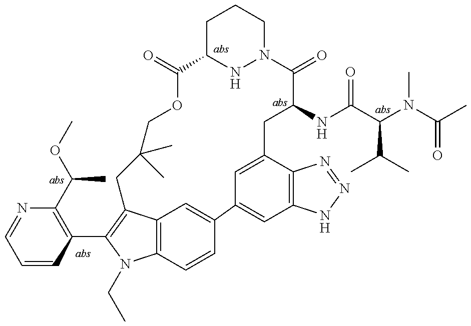 |
| B163 | 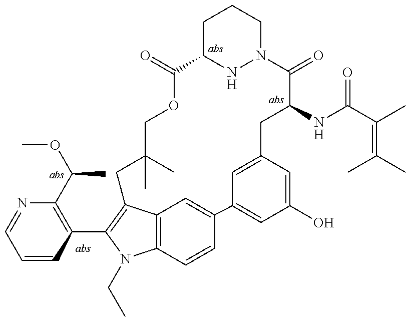 |
| B164 | 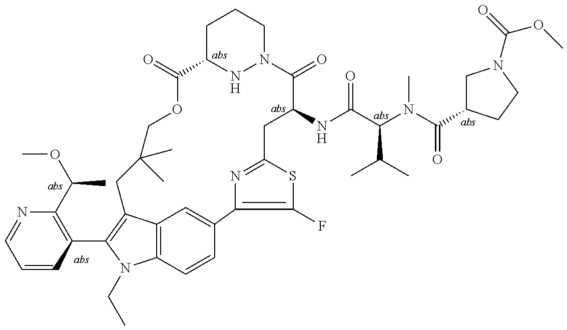 |
| B165 | 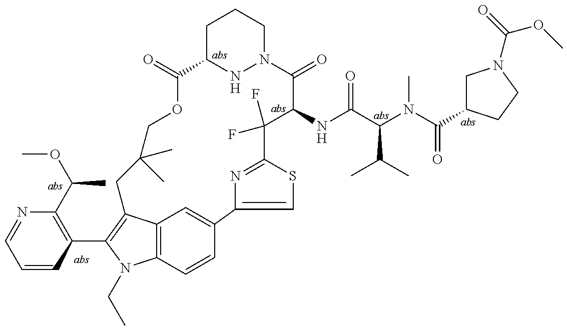 |
| B166 | 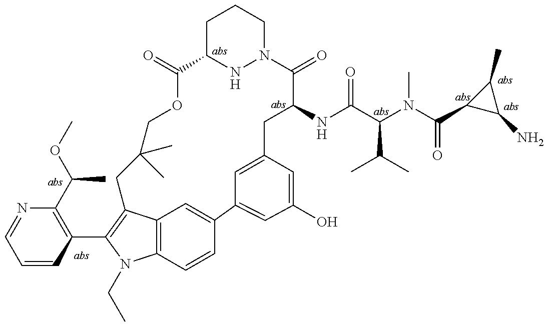 |
| B167 | 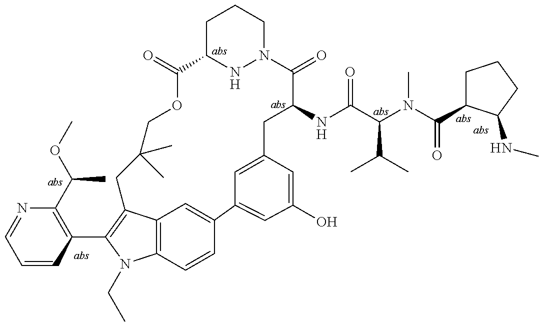 |
| B168 | 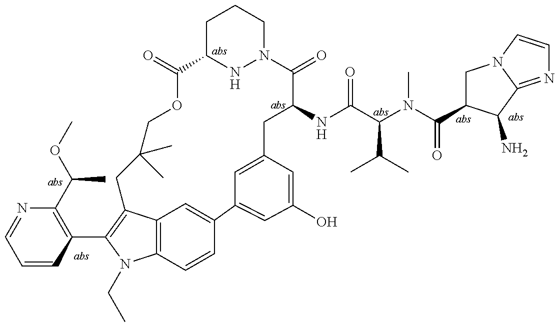 |
| B169 | 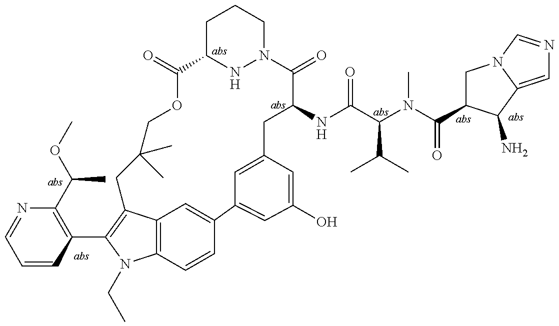 |
| B170 | 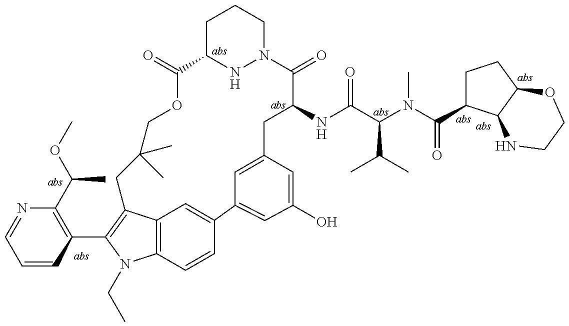 |
| B171 | 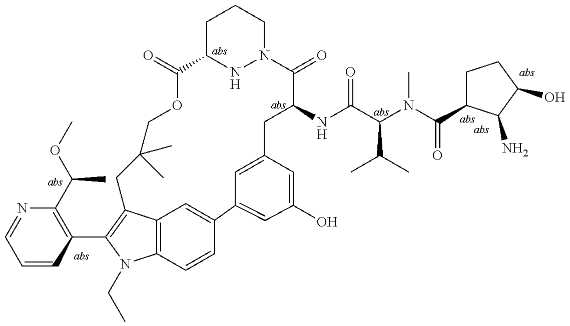 |
| B172 | 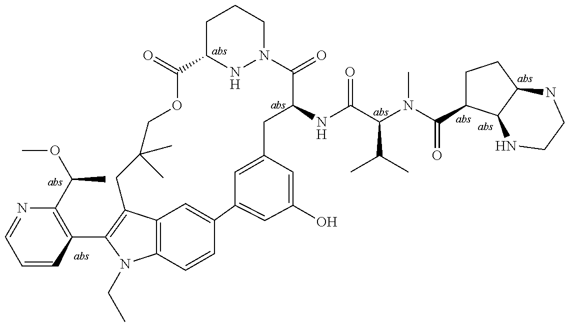 |
| B173 | 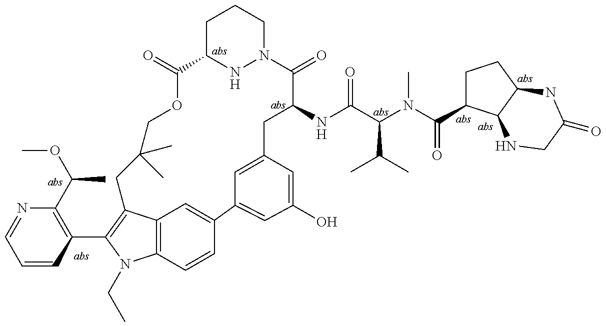 |
| B174 | 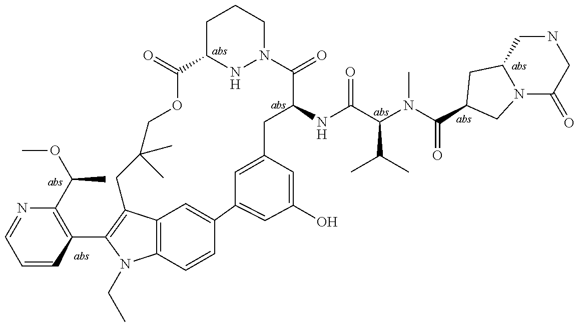 |
| B175 | 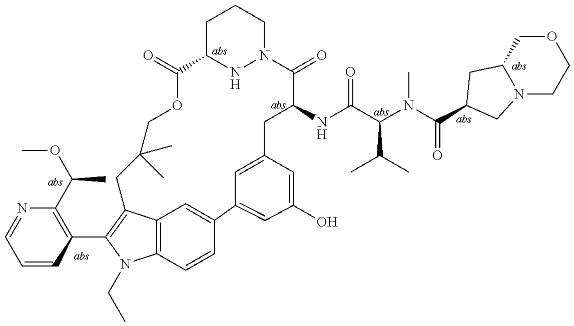 |
| B176 | 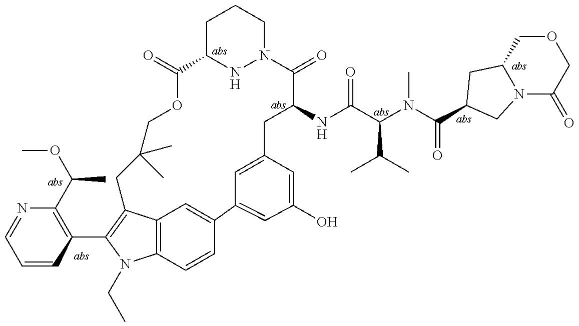 |
| B177 | 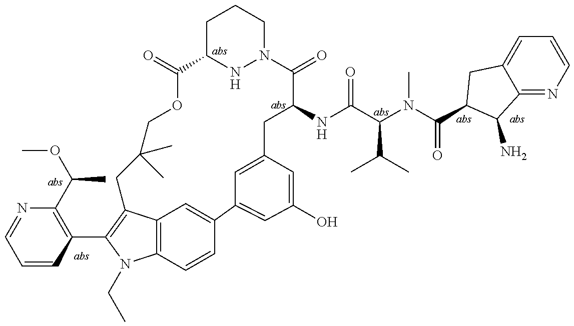 |
| B178 | 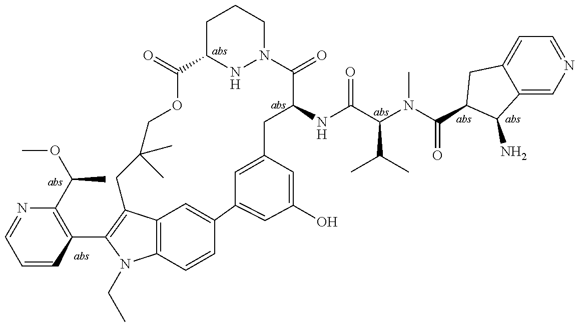 |
| B179 | 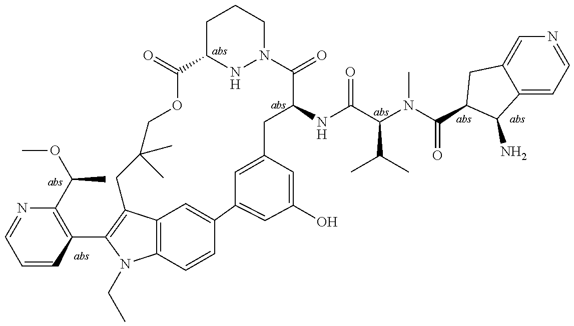 |
| B180 | 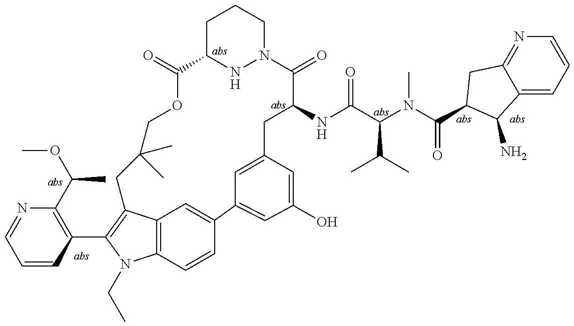 |
| B181 | 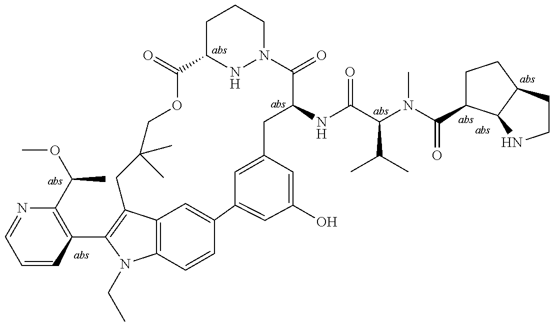 |
| B182 | 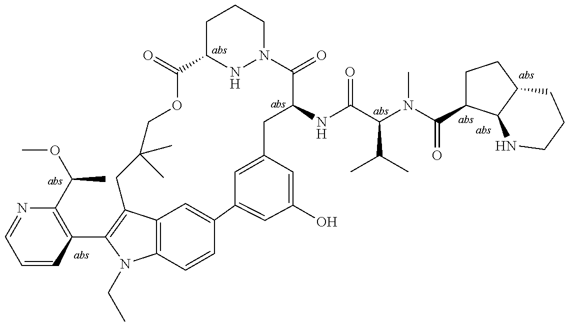 |
| B183 | 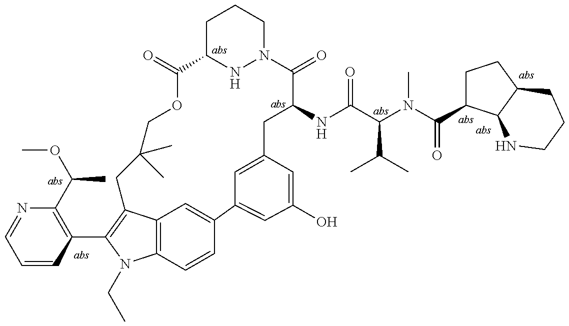 |
| B184 | 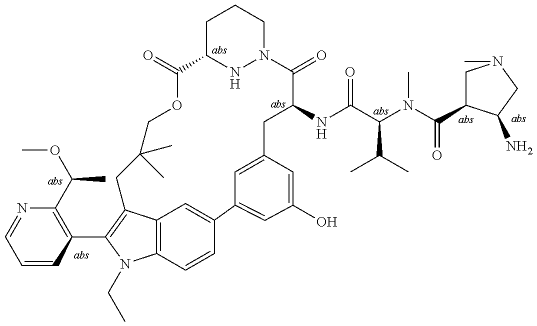 |
| B185 | 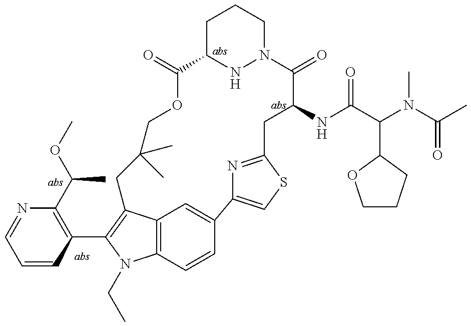 |
| B186 | 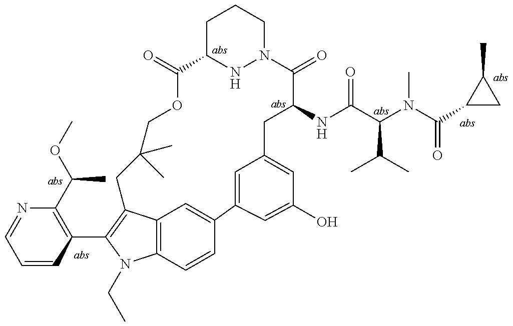 |
| B187 | 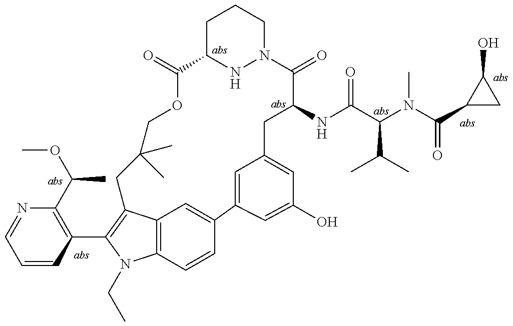 |
| B188 | 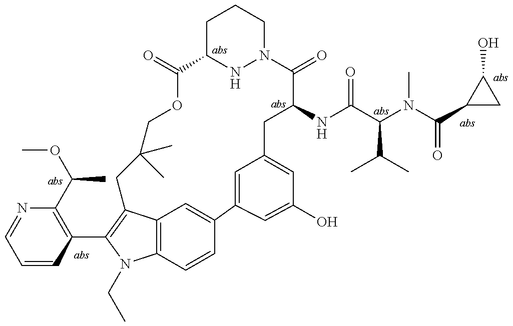 |
| B189 | 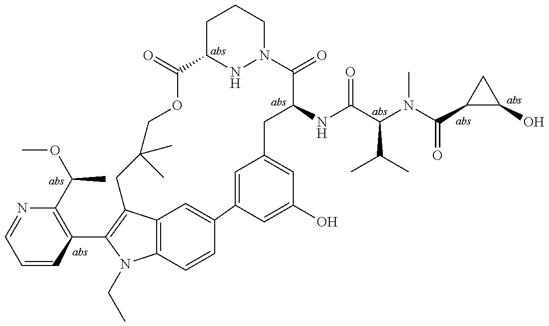 |
| B190 | 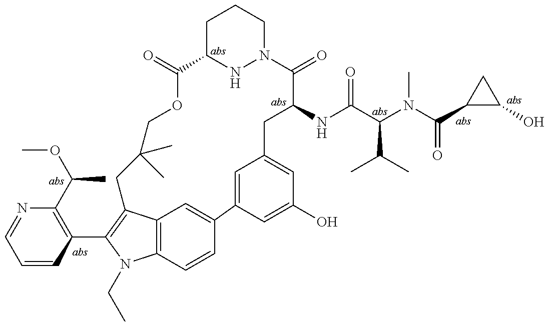 |
| B191 | 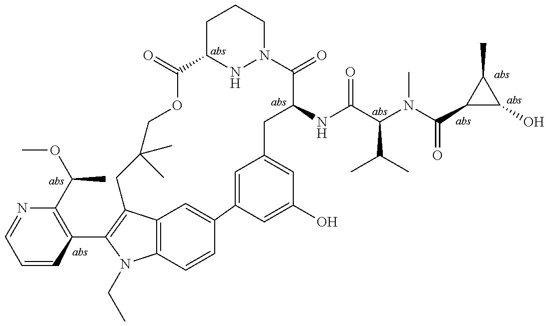 |
| B192 | 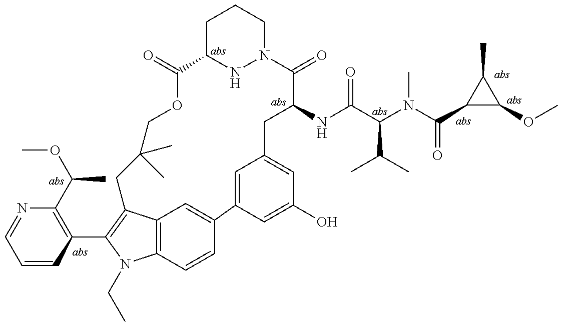 |
| B193 | 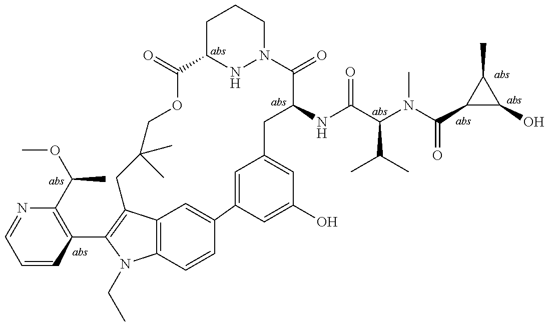 |
| B194 | 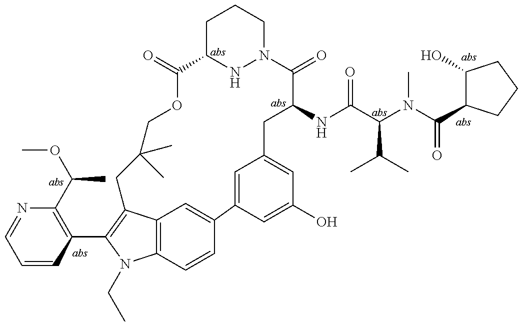 |
| B195 | 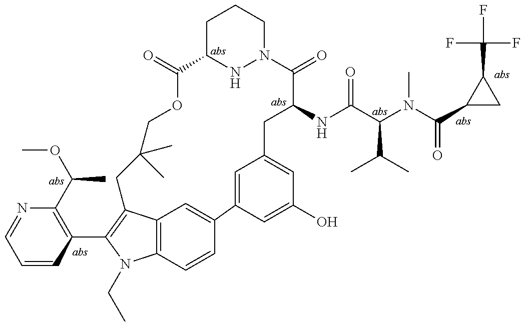 |
| B196 | 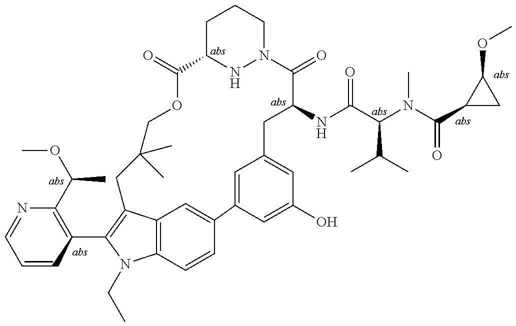 |
| B197 | 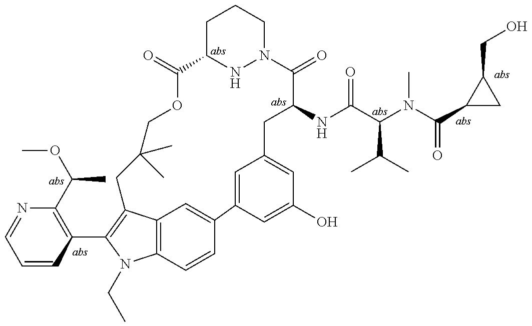 |
| B198 | 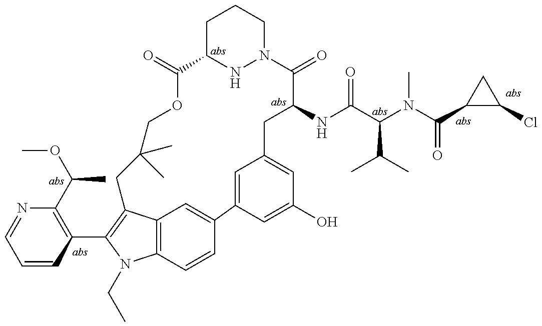 |
| B199 | 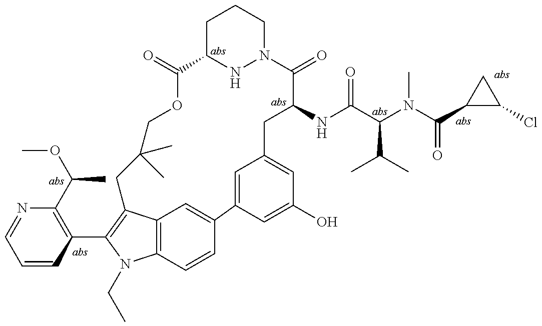 |
| B200 | 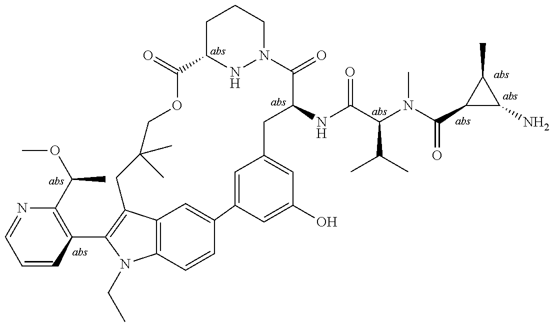 |
| B201 | 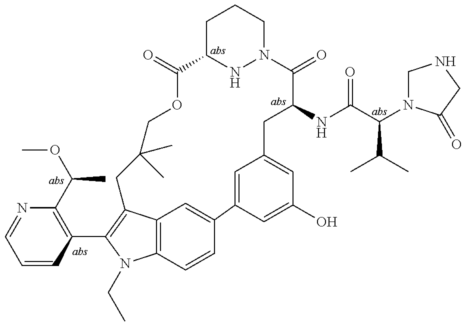 |
| B202 | 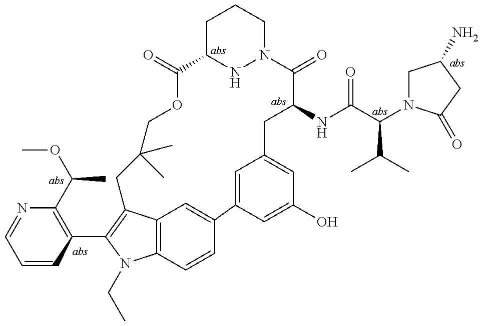 |
| B203 | 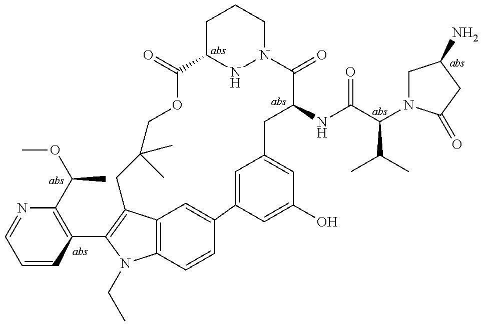 |
| B204 | 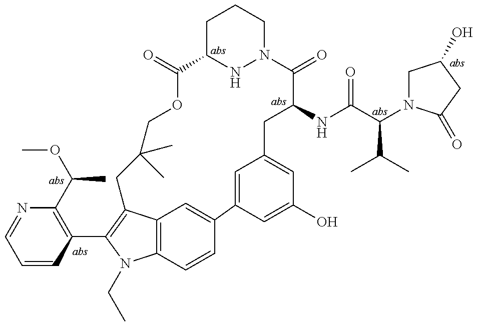 |
| B205 | 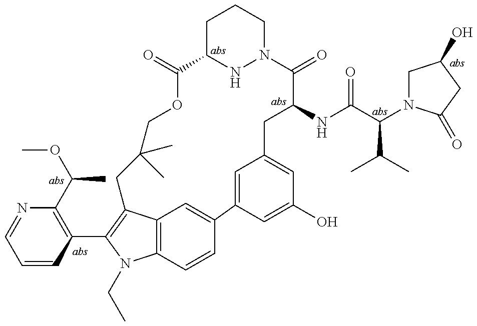 |
| B206 | 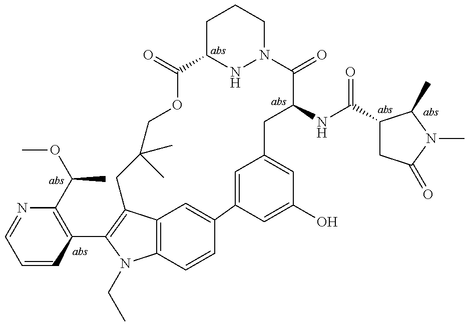 |
| B207 | 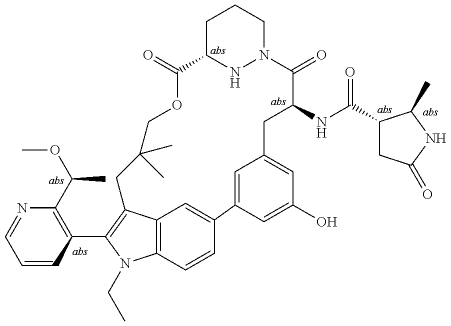 |
| B208 | 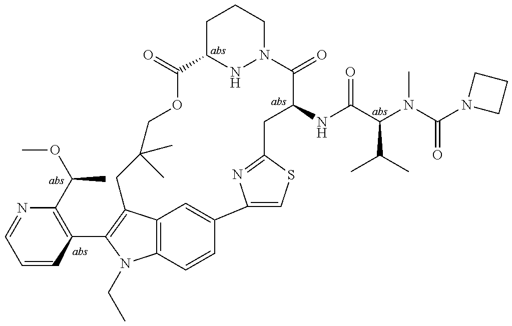 |
| B209 | 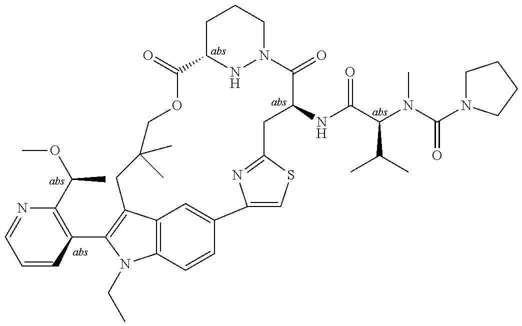 |
| B210 | 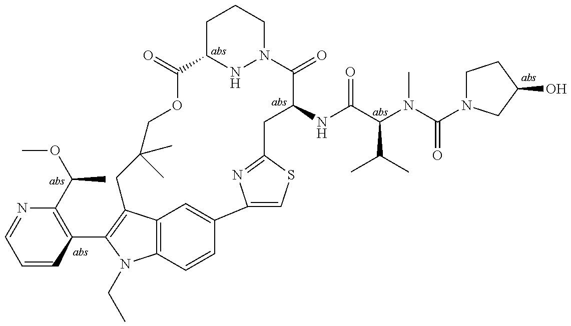 |
| B211 | 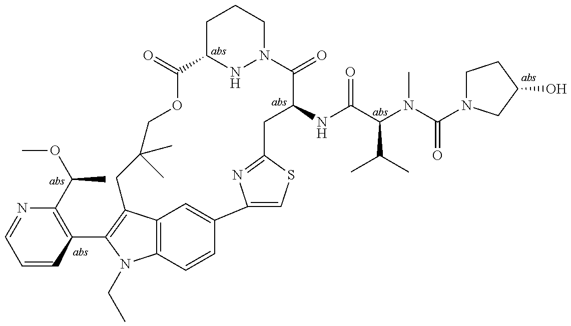 |
| B212 | 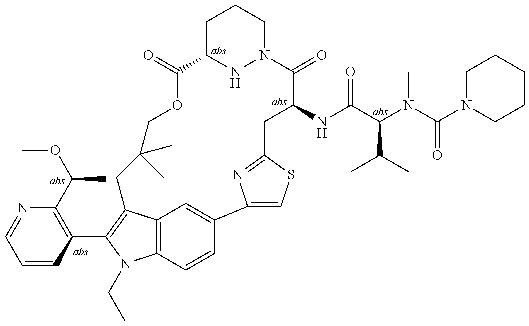 |
| B213 | 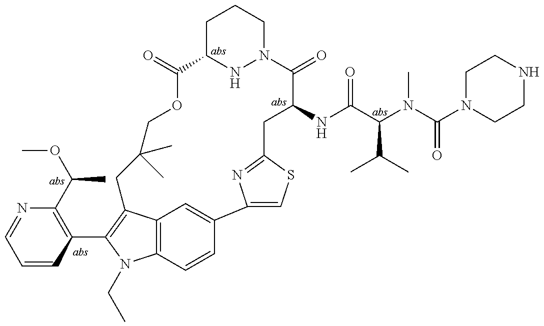 |
| B214 | 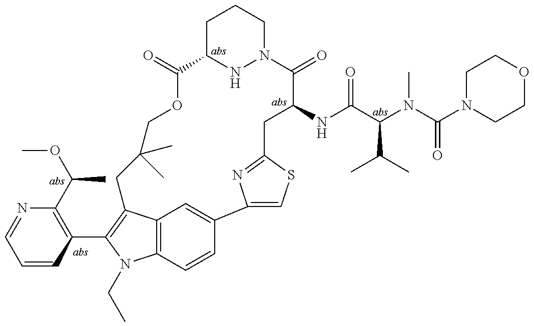 |
| B215 | 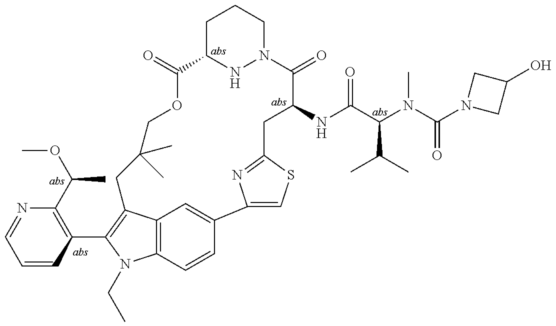 |
| B216 | 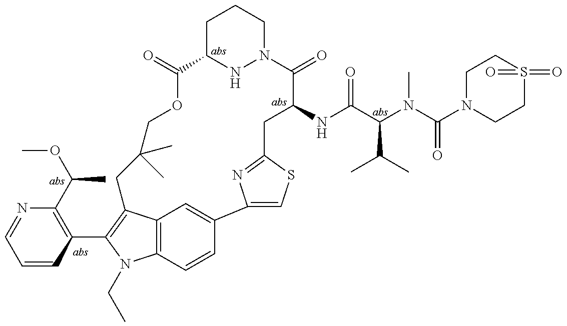 |
| B217 | 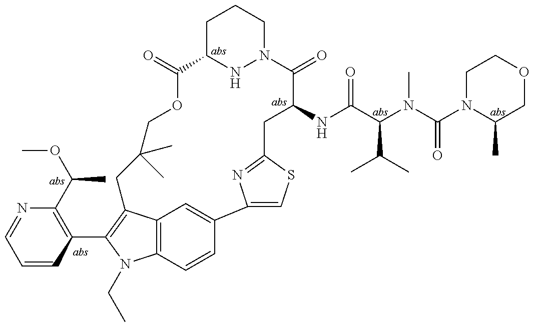 |
| B218 | 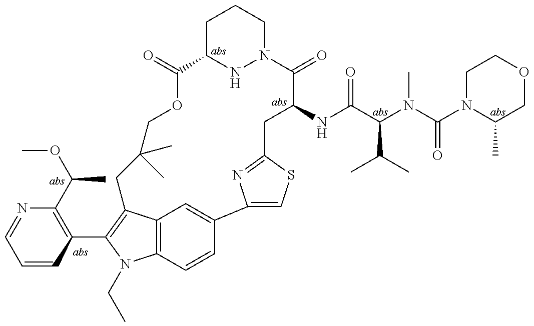 |
| B219 | 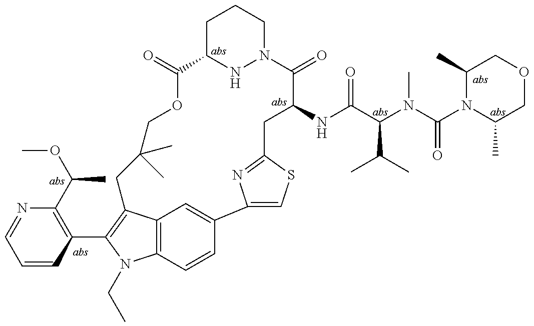 |
| B220 | 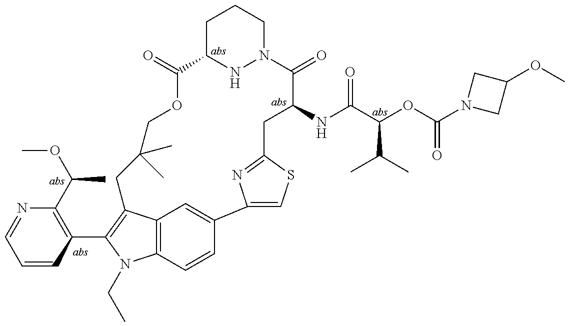 |
| B221 | 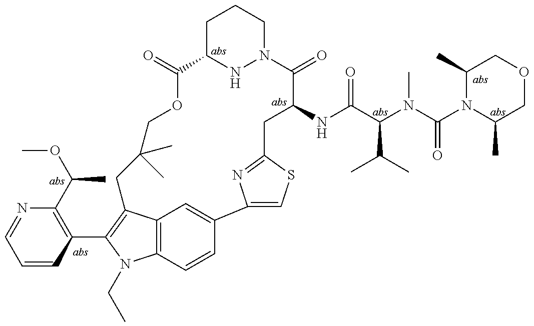 |
| B222 | 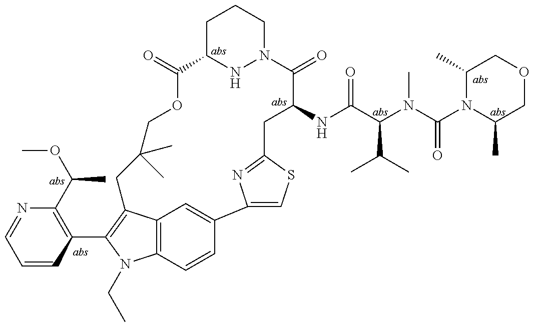 |
| B223 | 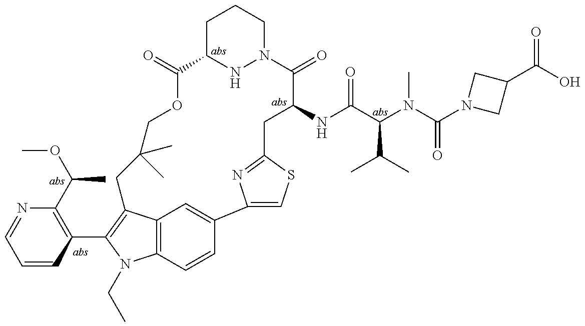 |
| B224 | 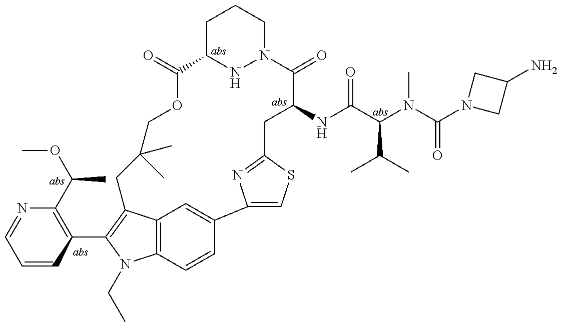 |
| B225 | 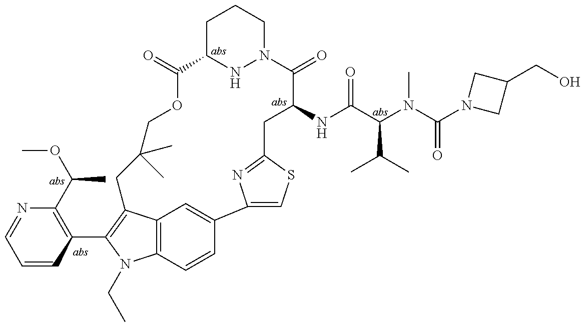 |
| B226 | 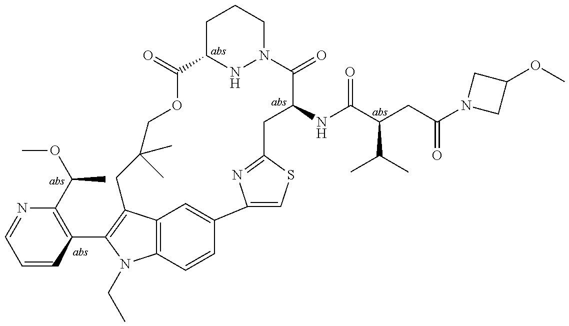 |
| B227 | 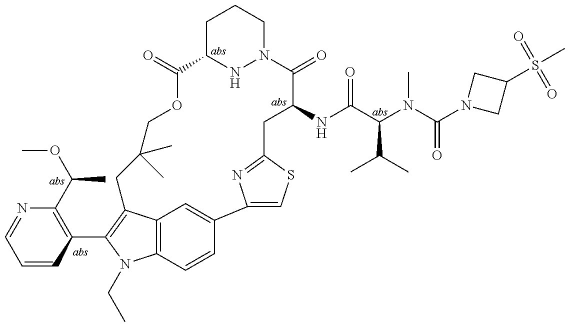 |
| B228 | 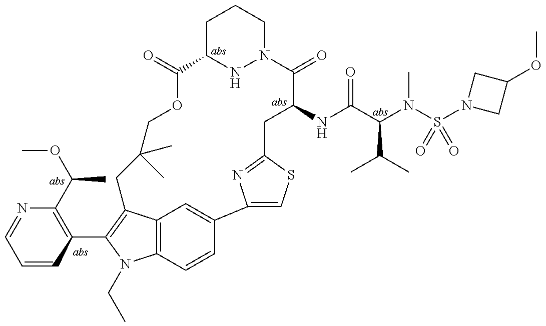 |
| B229 | 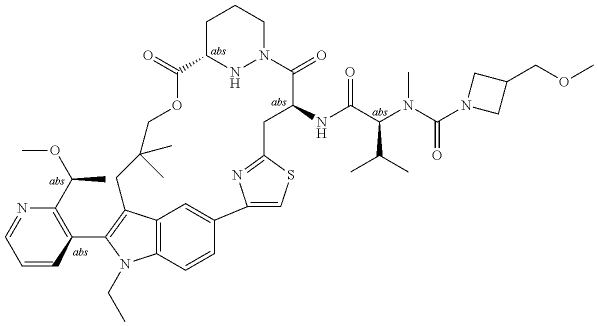 |
| B230 | 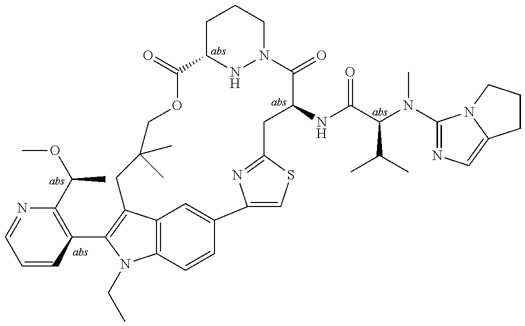 |
| B231 | 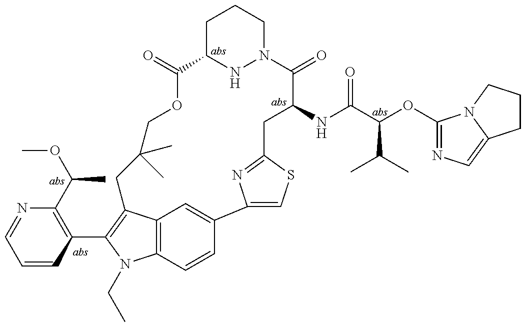 |
| B232 | 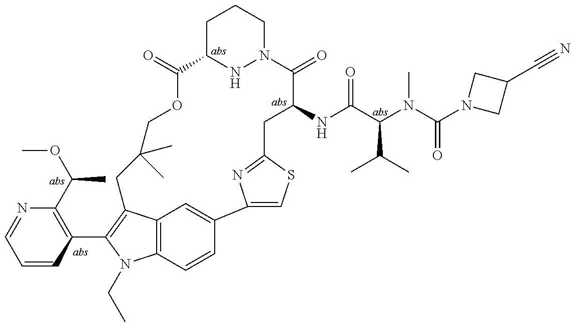 |
| B233 | 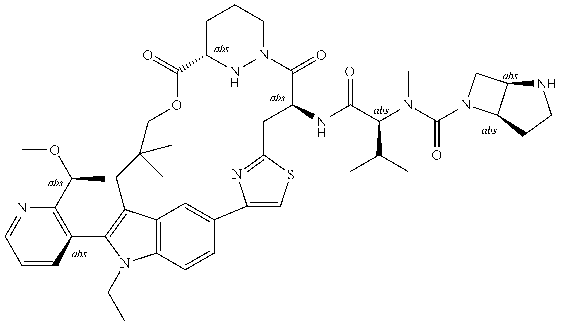 |
| B234 | 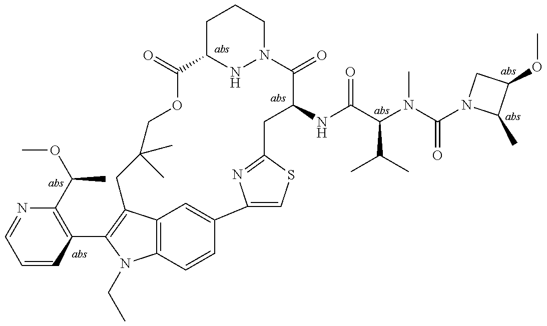 |
| B235 | 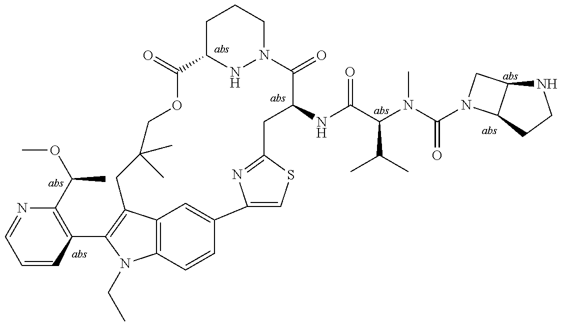 |
| B236 | 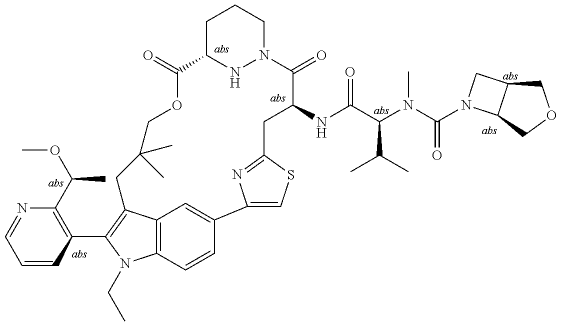 |
| B237 | 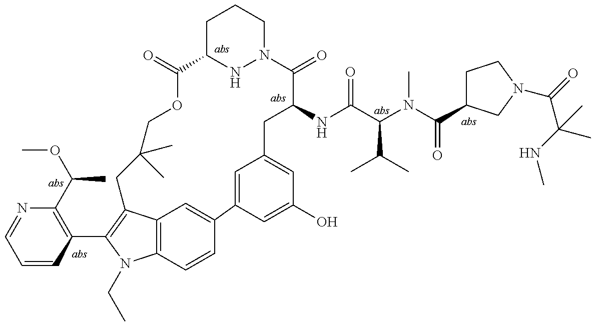 |
| B238 | 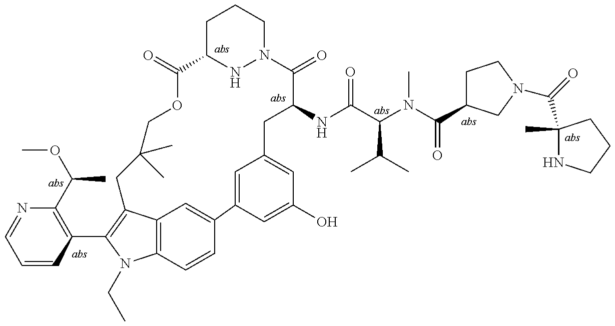 |
| B239 | 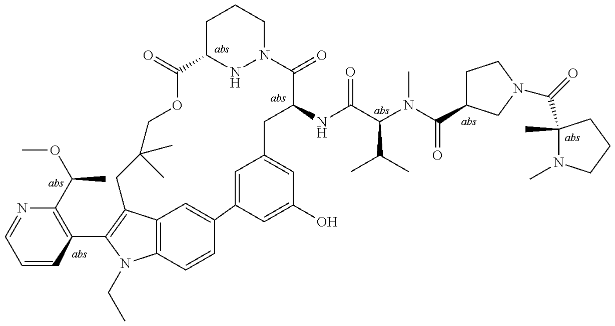 |
| B240 | 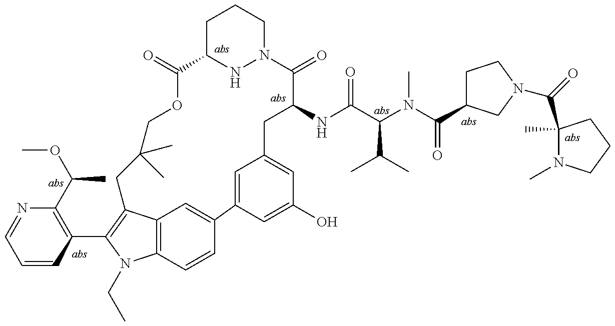 |
| B241 | 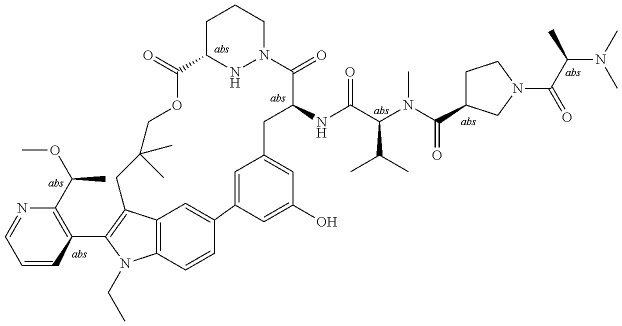 |
| B242 | 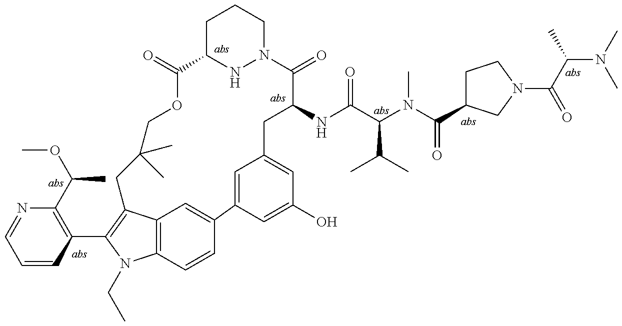 |
| B243 | 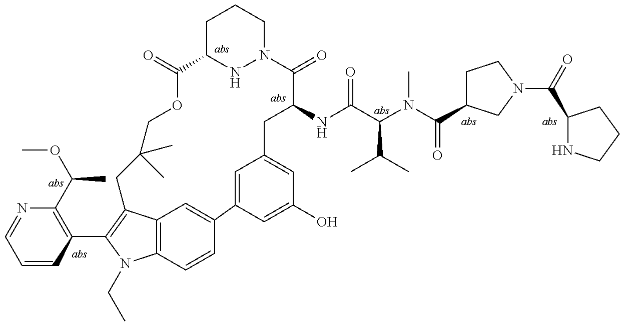 |
| B244 | 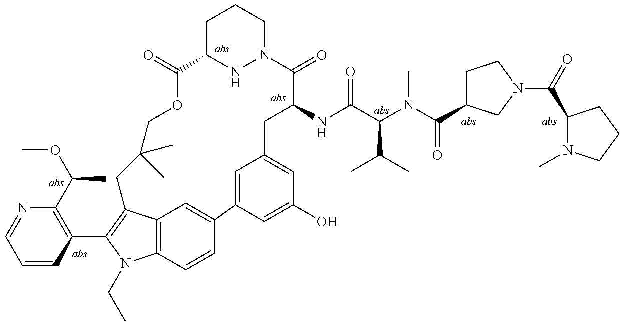 |
| B245 | 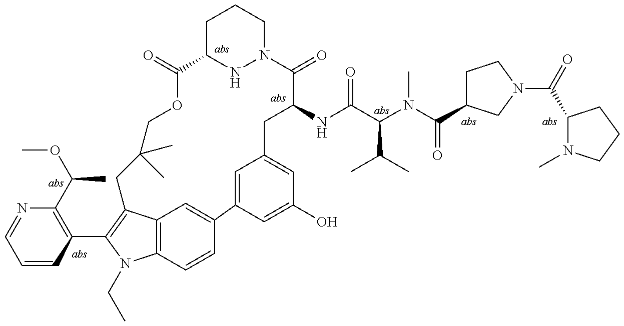 |
| B246 | 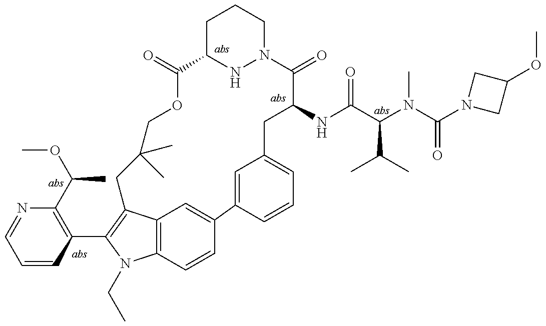 |
| B247 | 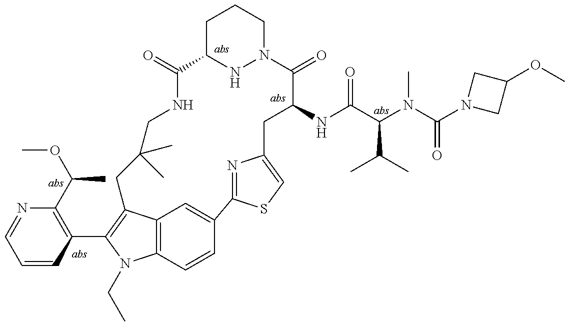 |
| B248 | 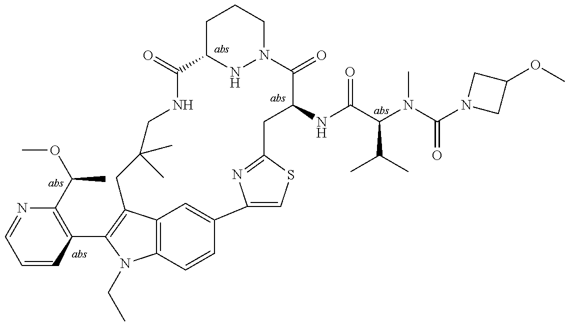 |
| B249 | 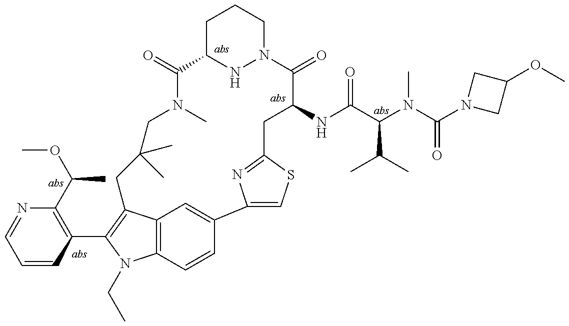 |
| B250 | 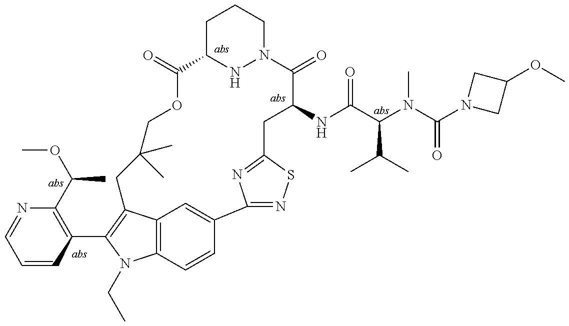 |
| B251 | 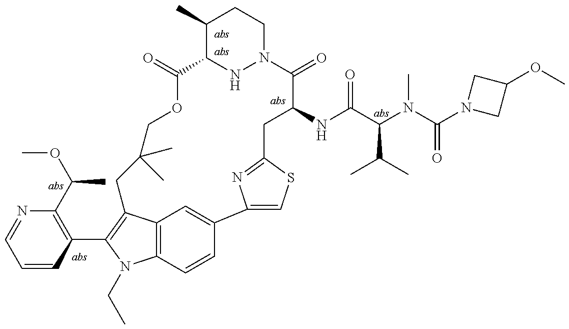 |
| B252 | 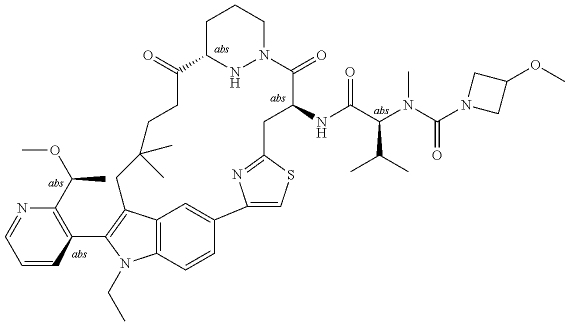 |
| B253 | 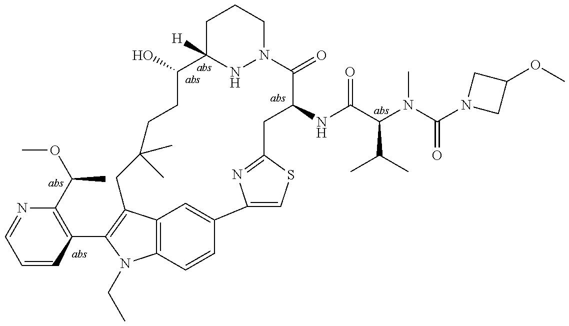 |
| B254 | 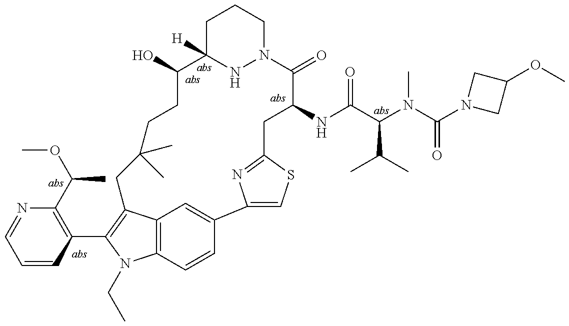 |
| B255 | 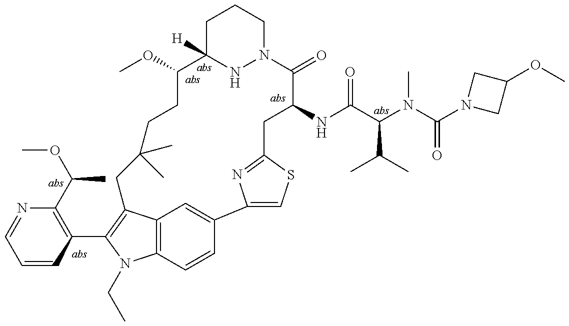 |
| B256 | 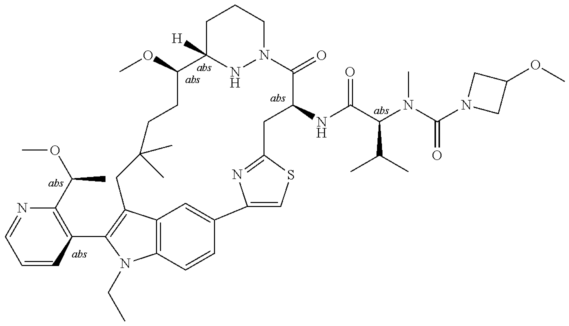 |
| B257 | 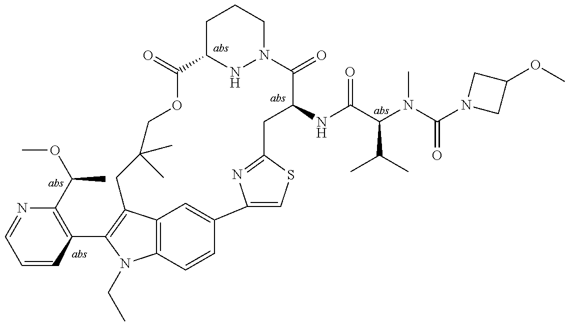 |
| B258 | 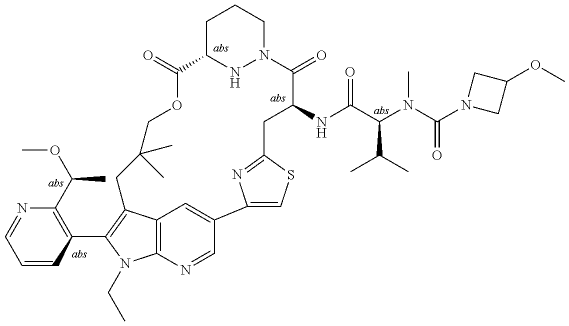 |
| B259 | 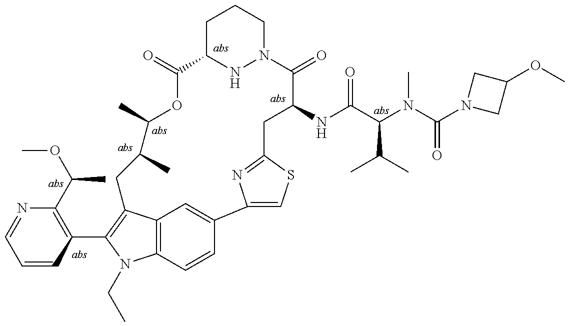 |
| B260 | 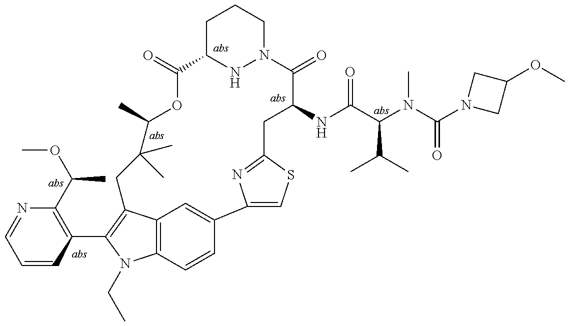 |
| B261 | 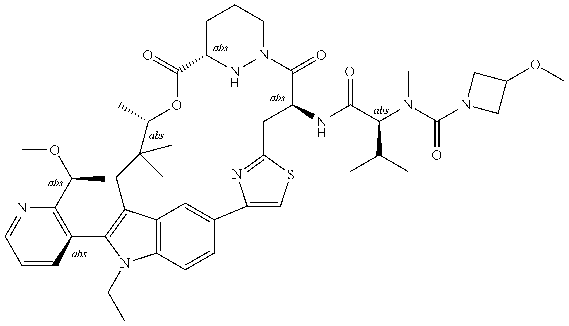 |
| B262 | 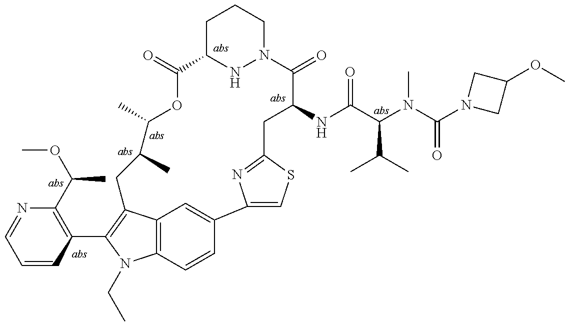 |
| B263 | 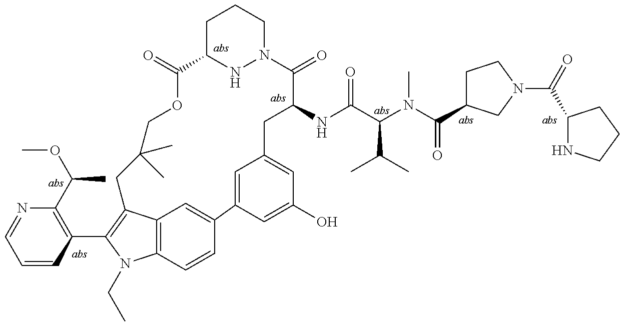 |

Note that some compounds are shown with bonds as flat or wedged. In some instances, the relative sterochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as K-Ras G12C, K-Ras G12D, K-Ras G12V, K-Ras G12S, K-Ras G13C, K-Ras G13D, or K-Ras Q61L. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras G12C, K-Ras G12D, K-Ras G12V, K-Ras G12S, K-Ras G13C, K-Ras G13D, or K-Ras Q61L. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

Compounds of Table 1 herein were prepared using methods disclosed herein or were prepared using methods disclosed herein combined with the knowledge of one of skill in the art. Compounds of Table 2 may be prepared using methods disclosed herein or may be prepared using methods disclosed herein combined with the knowledge of one of skill in the art.

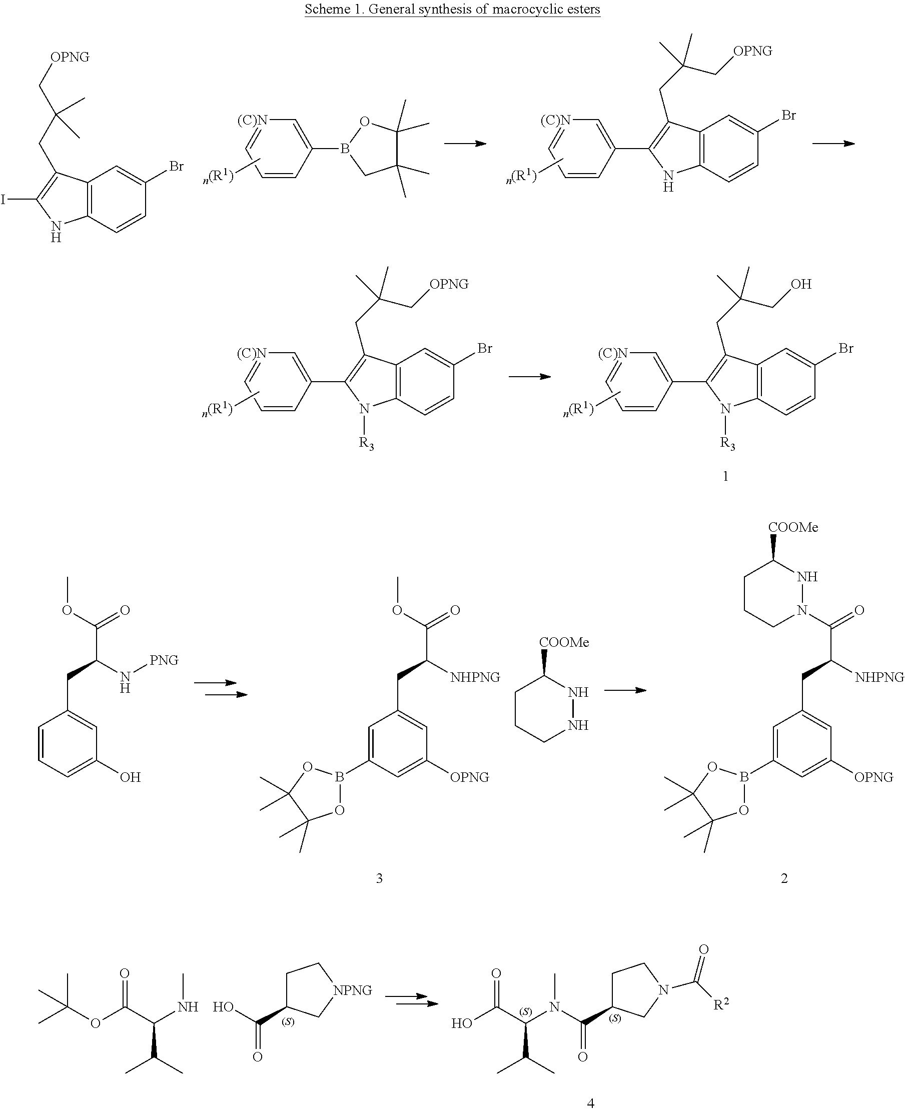

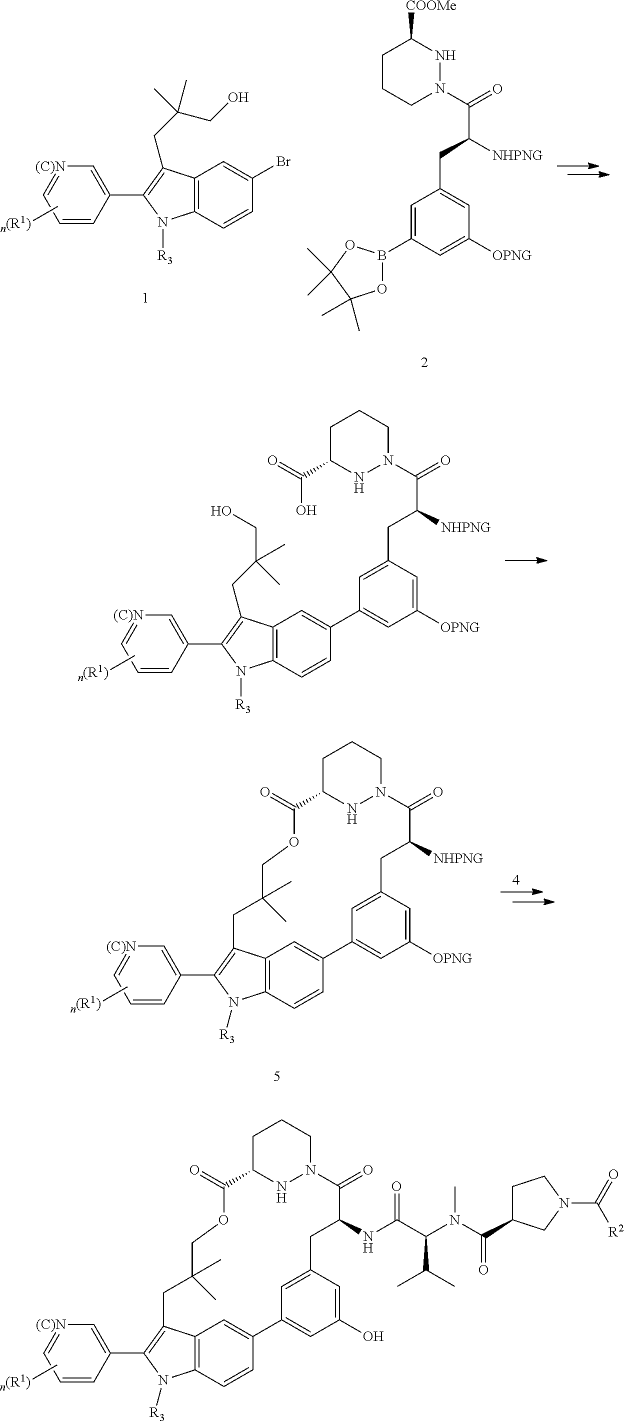

A general synthesis of macrocyclic esters is outlined in Scheme 1. An appropriately substituted Aryl Indole intermediate (1) can be prepared in three steps starting from protected 3-(5-bromo-2-iodo-1H-indol-3-yl)-2,2-dimethylpropan-1-ol and appropriately substituted boronic acid, including Palladium mediated coupling, alkylation, and de-protection reactions.

Methyl-amino-hexahydropyridazine-3-carboxylate-boronic ester (2) can be prepared in three steps, including protection, Iridium catalyst mediated borylation, and coupling with methyl (S)-hexahydropyridazine-3-carboxylate.

An appropriately substituted acetylpyrrolidine-3-carbonyl-N-methyl-L-valine (4) can be made by coupling of methyl-L-valinate and protected (S)-pyrrolidine-3-carboxylic acid, followed by deprotection, coupling with an appropriately substituted carboxylic acid, and a hydrolysis step.

The final macrocyclic esters can be made by coupling of methyl-amino-hexahydropyridazine-3-carboxylate-boronic ester (2) and intermediate (1) in the presence of Pd catalyst followed by hydrolysis and macrolactonization steps to result in an appropriately protected macrocyclic intermediate (5). Deprotection and coupling with an appropriately substituted acetylpyrrolidine-3-carbonyl-N-methyl-L-valine (4) results in a macrocyclic product. Additional deprotection or functionalization steps are be required to produce a final compound. For example, a person of skill in the art would be able to install into a macrocyclic ester a desired —B-L-W group of a compound of Formula (I), where B, L and W are defined herein, including by using methods exemplified in the Example section herein.

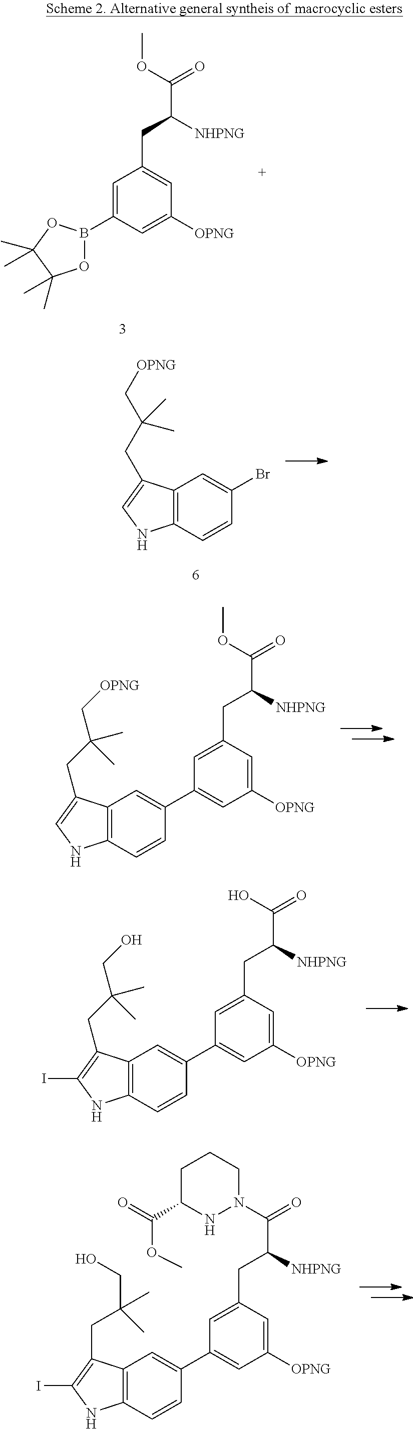

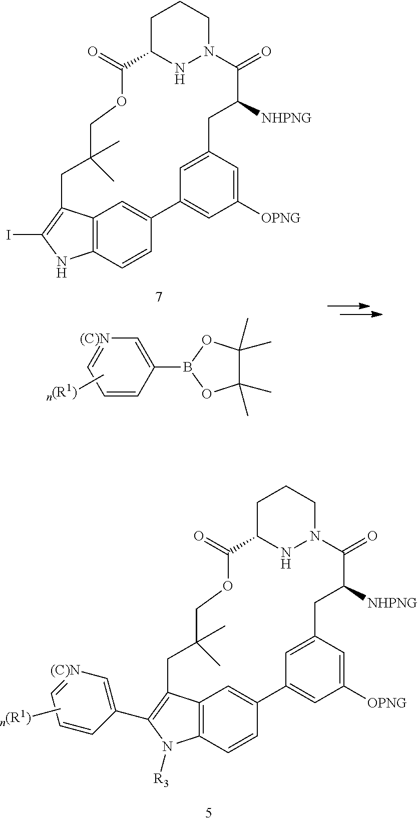

Alternatively, macrocyclic esters can be prepared as described in Scheme 2. An appropriately protected bromo-indolyl (6) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of Pd catalyst with an appropriately substituted boronic ester and alkylation can yield fully a protected macrocycle (5). Additional deprotection or functionalization steps are required to produce a final compound. For example, a person of skill in the art would be able to install into a macrocyclic ester a desired —B-L-W group of a compound of Formula (I), where B, L and W are defined herein, including by using methods exemplified in the Example section herein.

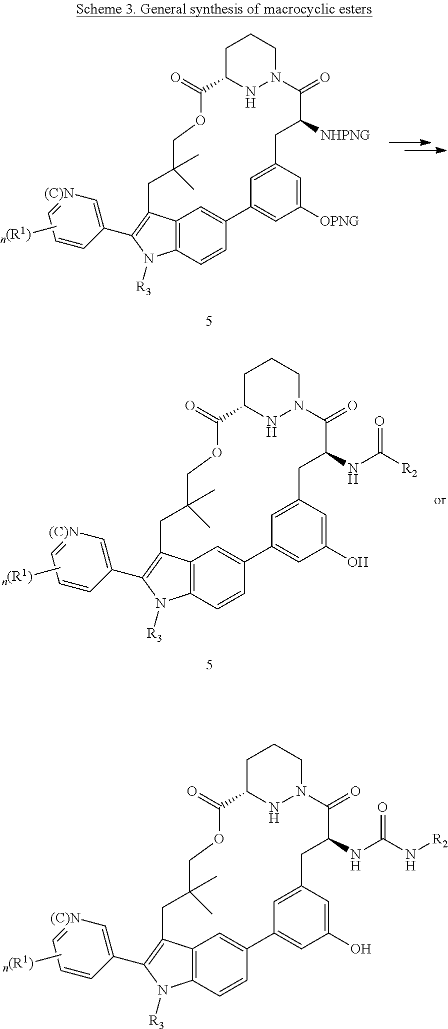

Alternatively, fully a protected macrocycle (5) can be deprotected and coupled with an appropriately substituted coupling partners, and deprotected to results in a macrocyclic product. Additional deprotection or functionalization steps are be required to produce a final compound. For example, a person of skill in the art would be able to install into a macrocyclic ester a desired —B-L-W group of a compound of Formula (I), where B, L and W are defined herein, including by using methods exemplified in the Example section herein.

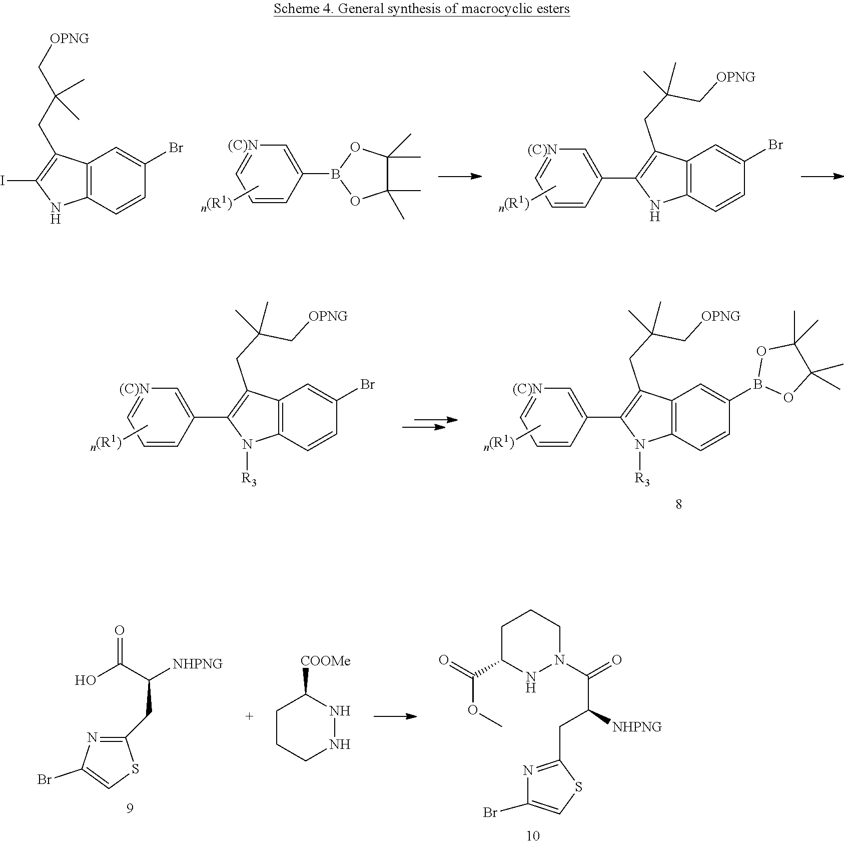

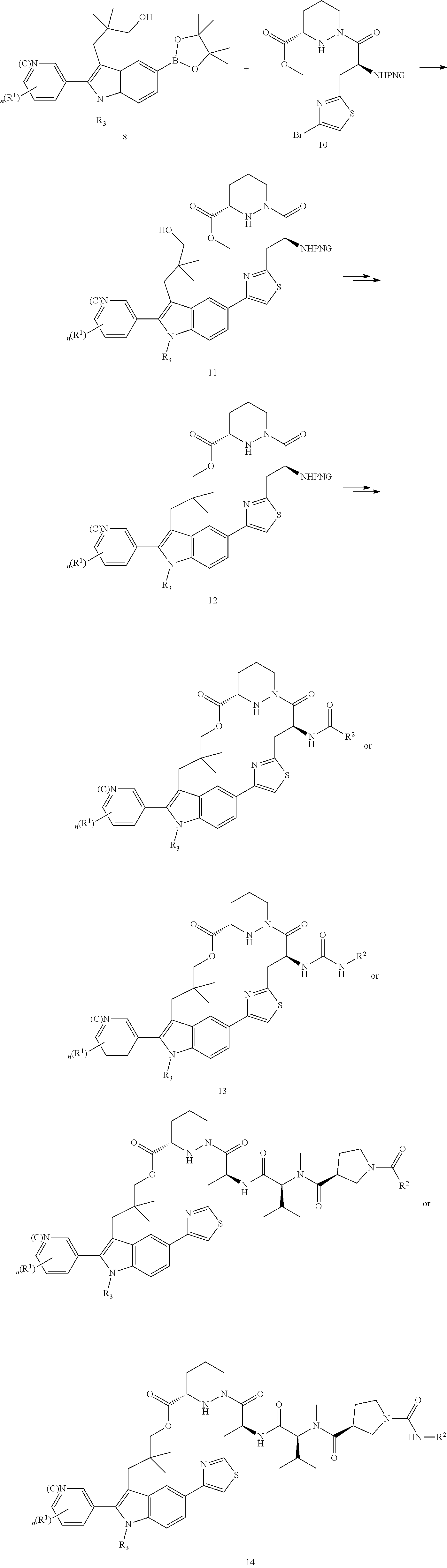

An alternative general synthesis of macrocyclic esters is outlined in Scheme 4. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted carboxylic acid (or other coupling partner) or intermediate 4 can result in a macrocyclic product. Additional deprotection or functionalization steps could be required to produce a final compound 13 or 14.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (I), where B, L and W are defined herein, including by using methods exemplified in the Example section herein.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Numbered Embodiments

[1] A compound, or pharmaceutically acceptable salt thereof, having the structure of Formula I:

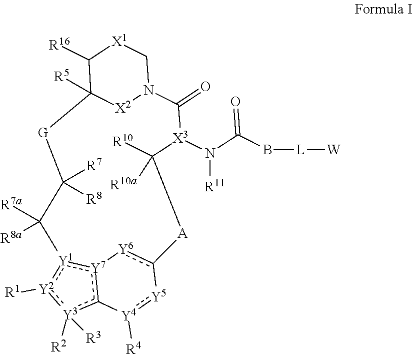

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;

L is absent or a linker;

W is hydrogen, cyano, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, optionally substituted 6 to 10-membered aryl, or optionally substituted 3 to 8-membered heteroaryl;

X¹ is optionally substituted C₁-C₂ alkylene, NR, O, or S(O)ₙ;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ is CH, CH₂, or N;

Y⁶ is C(O), CH, CH₂, or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or

R¹ and R² combine with the atoms to which they are attached to form an optionally substituted 3 to 14-membered heterocycloalkyl;

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7a} and R^{8a} are, independently, hydrogen, halo, optionally substituted C₁-C₃ alkyl, or combine with the carbon to which they are attached to form a carbonyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R⁹ and L combine with the atoms to which they are attached to form an optionally substituted 3 to 14-membered heterocycloalkyl;

R^{9′} is hydrogen or optionally substituted C₁-C₆ alkyl;

R¹⁰ is hydrogen, halo, hydroxy, C₁-C₃ alkoxy, or C₁-C₃ alkyl;

R^{10a} is hydrogen or halo;

R¹¹ is hydrogen or C₁-C₃ alkyl; and

R¹⁶ is hydrogen or C₁-C₃ alkyl.

[2] The compound, or pharmaceutically acceptable salt thereof, of paragraph [1], wherein G is optionally substituted C₁-C₄ heteroalkylene.

[3] The compound, or pharmaceutically acceptable salt thereof, of paragraph [1] or [2], wherein the compound has the structure of Formula Ic:

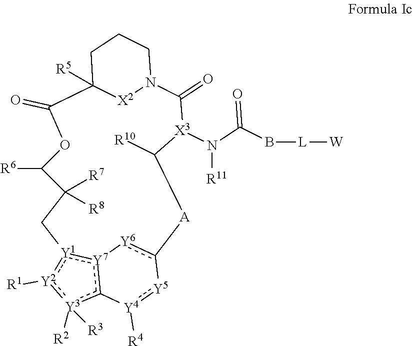

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

X² is O or NH;

X³ is N or CH;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

R¹⁰ is hydrogen, hydroxy, C₁-C₃ alkoxy, or C₁-C₃ alkyl; and

R¹¹ is hydrogen or C₁-C₃ alkyl.

[4] The compound, or pharmaceutically acceptable salt thereof, of any one of paragraphs [1] to [3], wherein X² is NH.

[5] The compound, or pharmaceutically acceptable salt thereof, of any one of paragraphs [1] to [4], wherein X³ is CH.

[6] The compound, or pharmaceutically acceptable salt thereof, of any one of paragraphs [1] to [5], wherein R¹¹ is hydrogen.

[7] The compound, or pharmaceutically acceptable salt thereof, of any one of paragraphs [1] to [5], wherein R¹¹ is C₁-C₃ alkyl.

[8] The compound, or pharmaceutically acceptable salt thereof, of paragraph [7], wherein R¹¹ is methyl.

[9] The compound, or pharmaceutically acceptable salt thereof, of any one of paragraphs [1] to [6], wherein the compound has the structure of Formula Id:

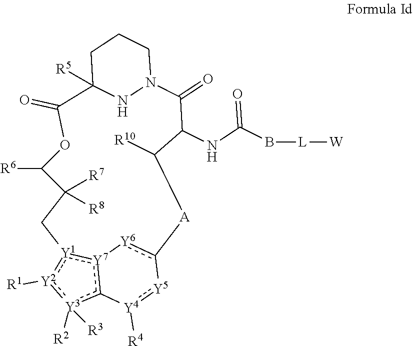

wherein the dotted lines represent zero, one, two, three, or four non-adjacent double bonds;

A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

n is 0, 1, or 2;

R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;

each R′ is, independently, H or optionally substituted C₁-C₄ alkyl;

Y¹ is C, CH, or N;

Y², Y³, Y⁴, and Y⁷ are, independently, C or N;

Y⁵ and Y⁶ are, independently, CH or N;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;

R⁴ is absent, hydrogen, halogen, cyano, or methyl optionally substituted with 1 to 3 halogens;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

[10] The compound, or pharmaceutically acceptable salt thereof, of any one of paragraphs [1] to [9] wherein X¹ is optionally substituted C₁-C₂ alkylene.

[11] The compound, or pharmaceutically acceptable salt thereof, of paragraph [10], wherein X¹ is methylene.

[12] The compound, or pharmaceutically acceptable salt thereof, of any one of paragraphs [1] to [11], wherein R⁵ is hydrogen.

[13] The compound, or pharmaceutically acceptable salt thereof, of any one of paragraphs [1] to [11], wherein R⁵ is C₁-C₄ alkyl optionally substituted with halogen.

[14] The compound, or pharmaceutically acceptable salt thereof, of paragraph [13], wherein R⁵ is methyl.

[15] The compound, or pharmaceutically acceptable salt thereof, of any one of paragraphs [1] to [14], wherein Y⁴ is C.

[16] The compound, or pharmaceutically acceptable salt thereof, of any one of paragraphs [1] to [15], wherein R⁴ is hydrogen.

[17] The compound, or pharmaceutically acceptable salt thereof, of any one of paragraphs [1] to [16], wherein Y⁵ is CH.

[18] The compound, or pharmaceutically acceptable salt thereof, of any one of paragraphs [1] to [17], wherein Y⁶ is CH.

[19] The compound, or pharmaceutically acceptable salt thereof, of any one of paragraphs [1] to [18], wherein Y¹ is C.

[20] The compound, or pharmaceutically acceptable salt thereof, of any one of paragraphs [1] to [19], wherein Y² is C.

[21] The compound, or pharmaceutically acceptable salt thereof, of any one of paragraphs [1] to [20], wherein Y³ is N.

[22] The compound, or pharmaceutically acceptable salt thereof, of any one of paragraphs [1] to [21], wherein R³ is absent.

[23] The compound, or pharmaceutically acceptable salt thereof, of any one of paragraphs [1] to [22], wherein Y⁷ is C.

[24] The compound, or pharmaceutically acceptable salt thereof, of any one of paragraphs [1] to [6] or [9] to [23], wherein the compound has the structure of Formula Ie:

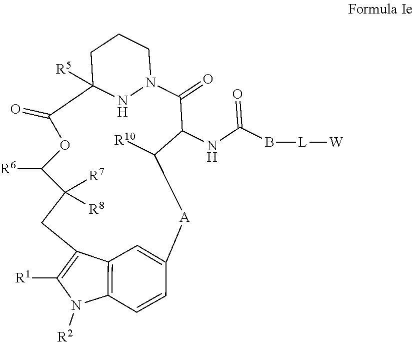

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;

R⁵ is hydrogen, C₁-C₄ alkyl optionally substituted with halogen, cyano, hydroxy, or C₁-C₄ alkoxy, cyclopropyl, or cyclobutyl;

R⁶ is hydrogen or methyl; R⁷ is hydrogen, halogen, or optionally substituted C₁-C₃ alkyl, or

R⁶ and R⁷ combine with the carbon atoms to which they are attached to form an optionally substituted 3 to 6-membered cycloalkyl or optionally substituted 3 to 7-membered heterocycloalkyl;

R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;

R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R^{7′} and R^{8′} combine with the carbon atom to which they are attached to form optionally substituted 3 to 6-membered cycloalkyl or optionally substituted 3 to 7-membered heterocycloalkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

[25] The compound, or pharmaceutically acceptable salt thereof, of any one of paragraphs [3] to [24], wherein R⁸ is hydrogen.

[26] The compound, or pharmaceutically acceptable salt thereof, of any one of paragraphs [1] to [25], wherein R² is hydrogen, cyano, optionally substituted C₁-C₆ alkyl, optionally substituted 3 to 6-membered cycloalkyl, or optionally substituted 3 to 6-membered heterocycloalkyl.

[27] The compound, or pharmaceutically acceptable salt thereof, of paragraph [26], wherein R² is optionally substituted C₁-C₆ alkyl.

[28] The compound, or pharmaceutically acceptable salt thereof, of paragraph [27], wherein R² is ethyl.

[29] The compound, or pharmaceutically acceptable salt thereof, of any one of paragraphs [1] to [28], wherein R⁷ is optionally substituted C₁-C₃ alkyl.

[30] The compound, or pharmaceutically acceptable salt thereof, of paragraph [29], wherein R⁷ is C₁-C₃ alkyl.

[31] The compound, or pharmaceutically acceptable salt thereof, of any one of paragraphs [1] to [30], wherein R⁸ is optionally substituted C₁-C₃ alkyl.

[32] The compound, or pharmaceutically acceptable salt thereof, of paragraph [31], wherein R⁸ is C₁-C₃ alkyl.

[33] The compound, or pharmaceutically acceptable salt thereof, of any one of paragraphs [1] to [32], wherein the compound has the structure of Formula If:

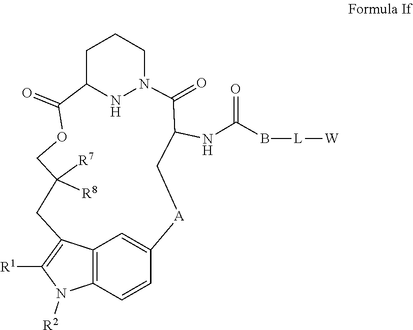

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₃ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;

R² is C₁-C₆ alkyl or 3 to 6-membered cycloalkyl;

R⁷ is C₁-C₃ alkyl;

R^{a} is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

[34] The compound, or pharmaceutically acceptable salt thereof, of any one of paragraphs [1] to [33], wherein R¹ is 5 to 10-membered heteroaryl.

[35] The compound, or pharmaceutically acceptable salt thereof, of paragraph [34], wherein R¹ is optionally substituted 6-membered aryl or optionally substituted 6-membered heteroaryl.

[36] The compound, or pharmaceutically acceptable salt thereof, of any one of paragraphs [1] to [35], wherein the compound has the structure of Formula Ig:

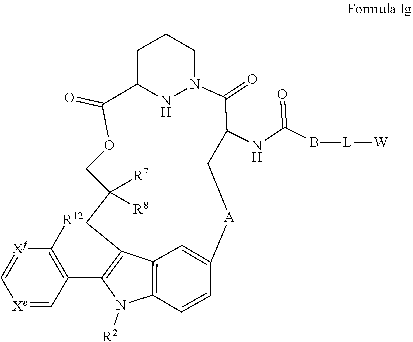

wherein A is, optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R² is C₁-C₆ alkyl or 3 to 6-membered cycloalkyl;

R⁷ is C₁-C₃ alkyl;

R⁸ is C₁-C₃ alkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} is N, CH, or CR¹⁷;

X^{f} is N or CH;

R¹² is optionally substituted C₁-C₆ alkyl or optionally substituted C₁-C₆ heteroalkyl; and

R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

[37] The compound, or pharmaceutically acceptable salt thereof, of paragraph [36], wherein X^{e} is N and X^{f} is CH.

[38] The compound, or pharmaceutically acceptable salt thereof, of paragraph [36], wherein X^{e} is CH and X^{f} is N.

[39] The compound, or pharmaceutically acceptable salt thereof, of paragraph [36], wherein X^{e} is CR¹⁷ and X^{f} is N.

[40] The compound, or pharmaceutically acceptable salt thereof, of any one of paragraphs [36] to [39], wherein R¹² is optionally substituted C₁-C₆ heteroalkyl.

[41] The compound, or pharmaceutically acceptable salt thereof, of any one of paragraphs [36] CH₃ to [40], wherein R¹² is

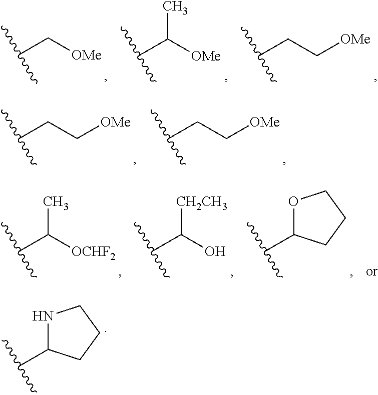

[42] The compound, or pharmaceutically acceptable salt thereof, of any one of paragraphs [1] to [41], wherein the compound has the structure of Formula Ih:

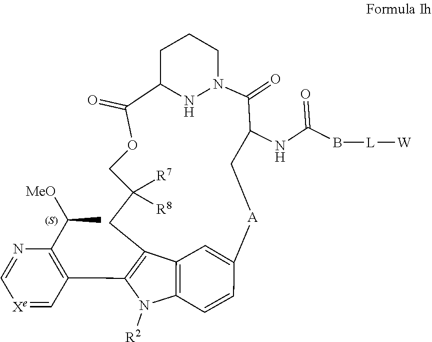

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R² is C₁-C₆ alkyl or 3 to 6-membered cycloalkyl;

R⁷ is C₁-C₃ alkyl;

R⁸ is C₁-C₃ alkyl;

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;

X^{e} is CH, or CR¹⁷; and

R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

[43] The compound, or pharmaceutically acceptable salt thereof, of any one of paragraphs [1] to [42], wherein the compound has the structure of Formula II:

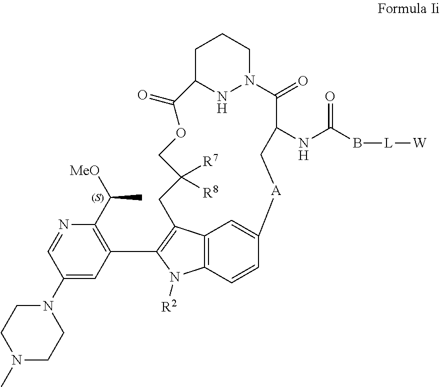

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;

L is absent or a linker;

W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;

R² is C₁-C₆ alkyl or 3 to 6-membered cycloalkyl;

R⁷ is C₁-C₃ alkyl;

R⁸ is C₁-C₃ alkyl; and

R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

[44] The compound, or pharmaceutically acceptable salt thereof, of any one of paragraphs [1] to [43], wherein A is optionally substituted 6-membered arylene.

[45] The compound, or pharmaceutically acceptable salt thereof, of paragraph [44], wherein A has the structure:

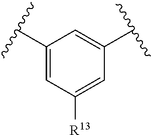

wherein R¹³ is hydrogen, hydroxy, amino, cyano, optionally substituted C₁-C₆ alkyl, or optionally substituted C₁-C₆ heteroalkyl.

[46] The compound, or pharmaceutically acceptable salt thereof, of paragraph [45], wherein R¹³ is hydrogen.

[47] The compound, or pharmaceutically acceptable salt thereof, of paragraph [45], wherein R¹³ is hydroxy.

[48] The compound, or pharmaceutically acceptable salt thereof, of any one of paragraphs [1] to [43], wherein A is optionally substituted 5 to 6-membered heteroarylene.

[49] The compound, or pharmaceutically acceptable salt thereof, of paragraph [48], wherein A is:

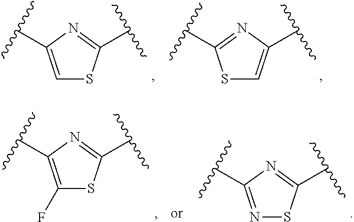 [SMILES: *c1csc(*)n1 ; *c1csc(*)n1 ; *c1nc(*)c(F)s1 ; *c1nsc(*)n1]

[50] The compound, or pharmaceutically acceptable salt thereof, of paragraph [49], wherein A is

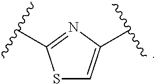 [SMILES: *c1csc(*)n1]

[51] The compound, or pharmaceutically acceptable salt thereof, of any one of paragraphs [1] to [50], wherein B is —CHR⁹—.

[52] The compound, or pharmaceutically acceptable salt thereof, of paragraph [51], wherein R⁹ is optionally substituted C₁-C₆ alkyl or optionally substituted 3 to 6-membered cycloalkyl.

[53] The compound, or pharmaceutically acceptable salt thereof, of paragraph [52], wherein R⁹ is:

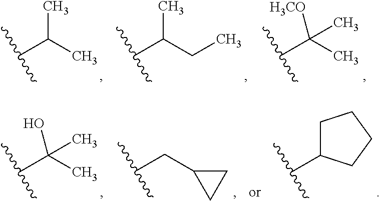

[54] The compound, or pharmaceutically acceptable salt thereof, of paragraph [53], wherein R⁹ is:

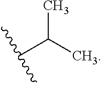 [SMILES: *C(C)C]

[55] The compound, or pharmaceutically acceptable salt thereof, of any one of paragraphs [1] to [50], wherein B is optionally substituted 6-membered arylene.

[56] The compound, or pharmaceutically acceptable salt thereof, of paragraph [55], wherein B is 6-membered arylene.

[57] The compound, or pharmaceutically acceptable salt thereof, of paragraph [56], wherein B is:

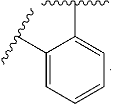 [SMILES: *c1ccccc1*]

[58] The compound, or pharmaceutically acceptable salt thereof, of any one of paragraphs [1] to [50], wherein B is absent.

[59] The compound, or pharmaceutically acceptable salt thereof, of any one of paragraphs [1] to [58], wherein R⁷ is methyl.

[60] The compound, or pharmaceutically acceptable salt thereof, of any one of paragraphs [1] to [59], wherein R⁸ is methyl.

[61] The compound, or pharmaceutically acceptable salt thereof, of any one of paragraphs [1] to [60], wherein the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to (B³)ᵢ—(C²)ⱼ—(B⁴)ₖ-A².

[62] The compound, or a pharmaceutically acceptable salt thereof, of any one of paragraphs [1] to [61], wherein the linker is acyclic.

[63] The compound, or a pharmaceutically acceptable salt thereof, of paragraph [62], wherein the linker has the structure of Formula IIa:

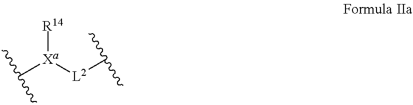

wherein X^{a} is absent or N;

R¹⁴ is absent, hydrogen, optionally substituted C₁-C₆ alkyl, or optionally substituted C₁-C₃ cycloalkyl; and

L² is absent, —C(O)—, —SO₂—, optionally substituted C₁-C₄ alkylene or optionally substituted C₁-C₄ heteroalkylene,

wherein at least one of X^{a}, R¹⁴, or L² is present.

[64] The compound, or a pharmaceutically acceptable salt thereof, of paragraph [63], wherein the linker has the structure:

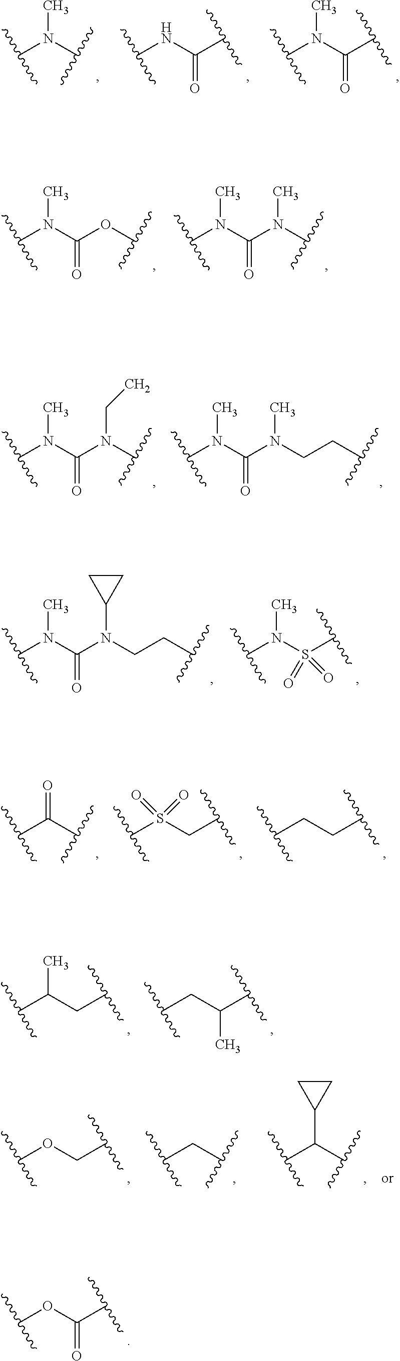

[65] The compound, or a pharmaceutically acceptable salt thereof, of paragraph [64], wherein the linker has the structure

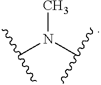 [SMILES: *N(*)C]

[66] The compound, or a pharmaceutically acceptable salt thereof, of any one of paragraphs [1] to [61], wherein the linker is or comprises a cyclic group.

[67] The compound, or a pharmaceutically acceptable salt thereof, of any one of paragraphs [1] to [61] or [66], wherein the linker has the structure of Formula IIb:

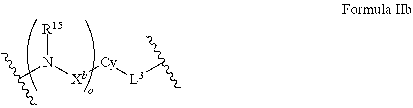

wherein o is 0 or 1;

X^{b} is C(O) or SO₂;

R¹⁵ is hydrogen or optionally substituted C₁-C₆ alkyl;

Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and

L³ is absent, —C(O)—, —SO₂—, optionally substituted C₁-C₄ alkylene or optionally substituted C₁-C₄ heteroalkylene.

[68] The compound, or a pharmaceutically acceptable salt thereof, of paragraph [67], wherein the linker has the structure:

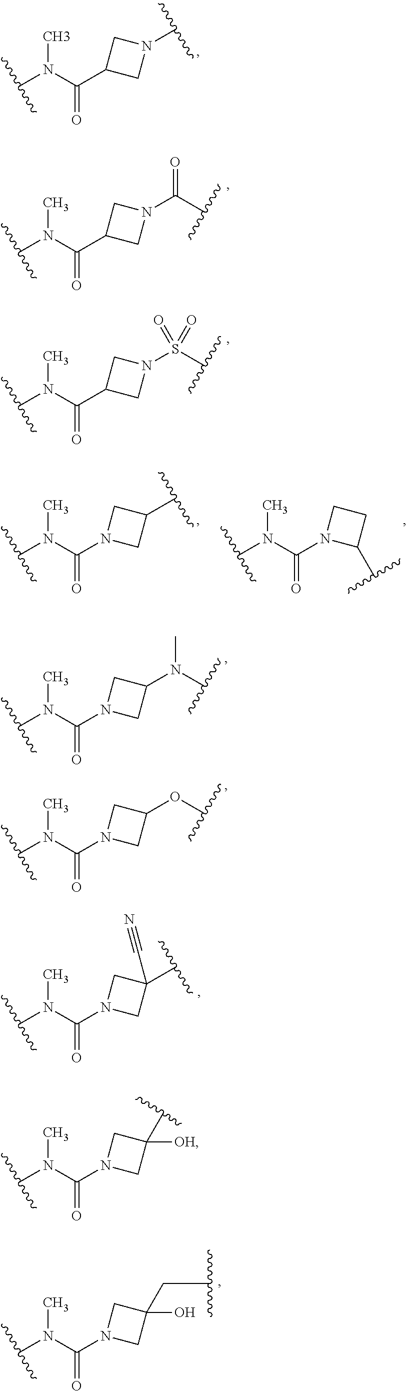

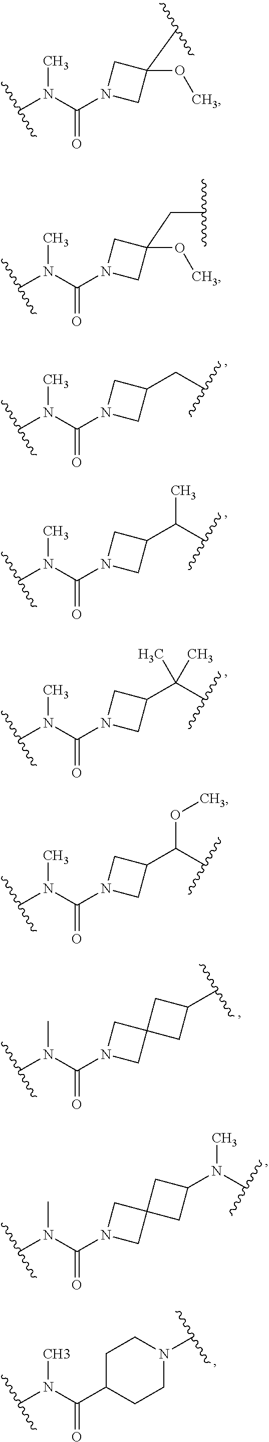

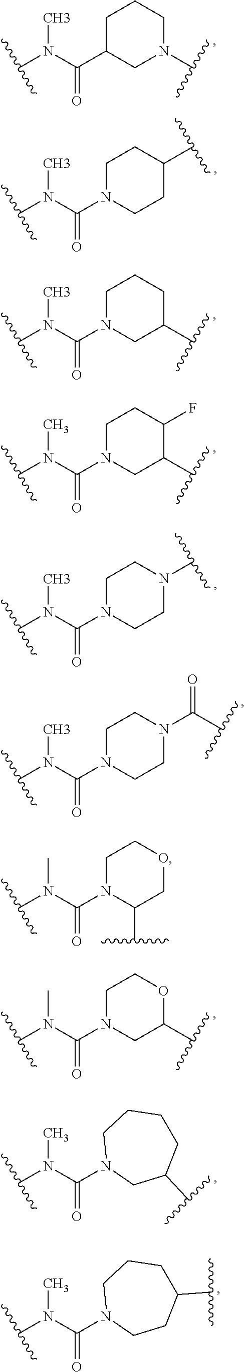

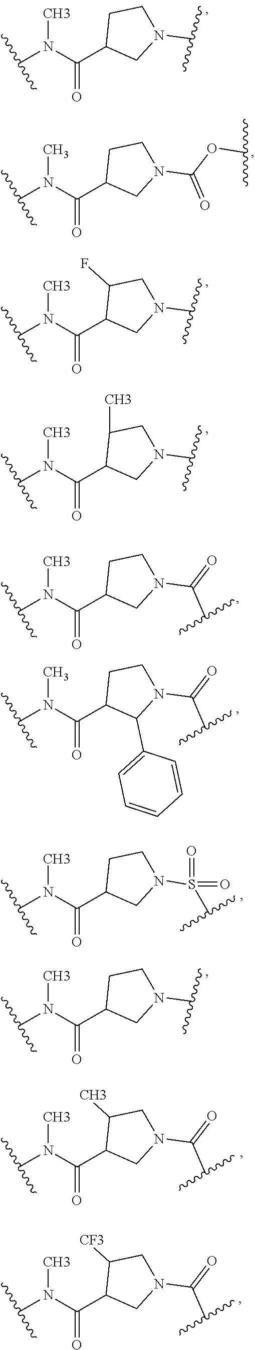

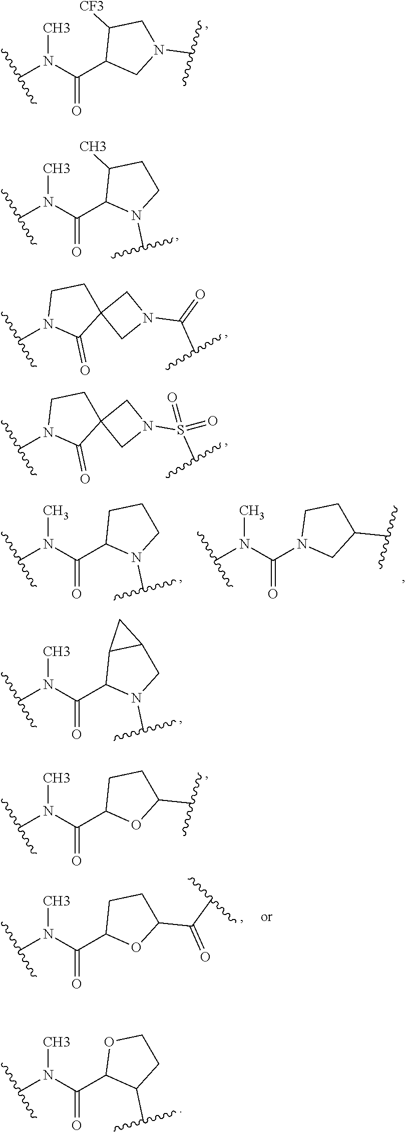

[69] The compound, or a pharmaceutically acceptable salt thereof, of any one of paragraphs [1] to [68], wherein W is hydrogen.

[70] The compound, or a pharmaceutically acceptable salt thereof, of any one of paragraphs [1] to [68], wherein W is optionally substituted amino.

[71] The compound, or a pharmaceutically acceptable salt thereof, of paragraph [70], wherein W is —NHCH₃ or —N(CH₃)₂.

[72] The compound, or a pharmaceutically acceptable salt thereof, of any one of paragraphs [1] to [68], wherein W is optionally substituted amido.

[73] The compound, or a pharmaceutically acceptable salt thereof, of paragraph [72], wherein W is

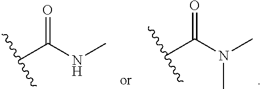

[74] The compound, or a pharmaceutically acceptable salt thereof, of any one of paragraphs [1] to [68], wherein W is optionally substituted C₁-C₄ alkoxy.

[75] The compound, or a pharmaceutically acceptable salt thereof, of paragraph [74], wherein W is methoxy or iso-propoxy.

[76] The compound, or a pharmaceutically acceptable salt thereof, of any one of paragraphs [1] to [68], wherein W is optionally substituted C₁-C₄ alkyl.

[77] The compound, or a pharmaceutically acceptable salt thereof, of paragraph [76], wherein W is methyl, ethyl, iso-propyl, tert-butyl, or benzyl.

[78] The compound, or a pharmaceutically acceptable salt thereof, of any one of paragraphs [1] to [68], wherein W is optionally substituted C₁-C₄ hydroxyalkyl.

[79] The compound, or a pharmaceutically acceptable salt thereof, of paragraph [78], wherein W is

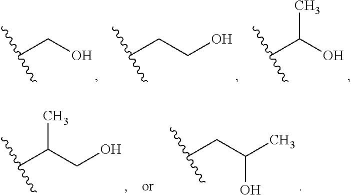

[80] The compound, or a pharmaceutically acceptable salt thereof, of any one of paragraphs [1] to [68], wherein W is optionally substituted C₁-C₄ aminoalkyl.

[81] The compound, or a pharmaceutically acceptable salt thereof, of paragraph [80], wherein W is

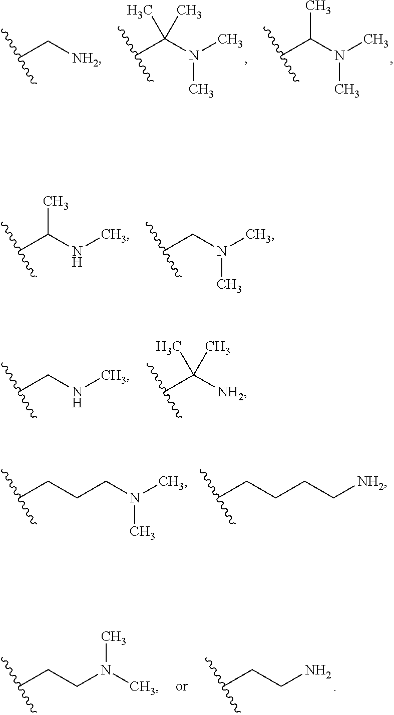

[82] The compound, or a pharmaceutically acceptable salt thereof, of any one of paragraphs [1] to [68], wherein W is optionally substituted C₁-C₄ haloalkyl.

[83] The compound, or a pharmaceutically acceptable salt thereof, of paragraph [82], wherein W is

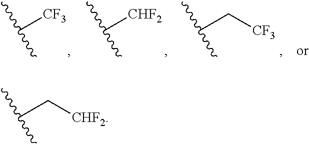

[84] The compound, or a pharmaceutically acceptable salt thereof, of any one of paragraphs [1] to [68], wherein W is optionally substituted C₁-C₄ guanidinoalkyl.

[85] The compound, or a pharmaceutically acceptable salt thereof, of paragraph [84], wherein W is

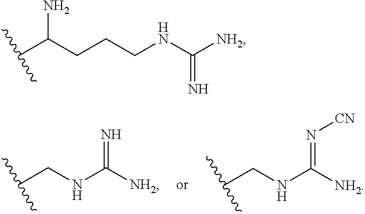 [SMILES: *C(N)CCCNC(=N)N ; *CNC(=N)N ; *CN/C(N)=N/C#N]

[86] The compound, or a pharmaceutically acceptable salt thereof, of any one of paragraphs [1] to [68], wherein W is C₀-C₄ alkyl optionally substituted 3 to 11-membered heterocycloalkyl.

[87] The compound, or a pharmaceutically acceptable salt thereof, of paragraph [86], wherein W is

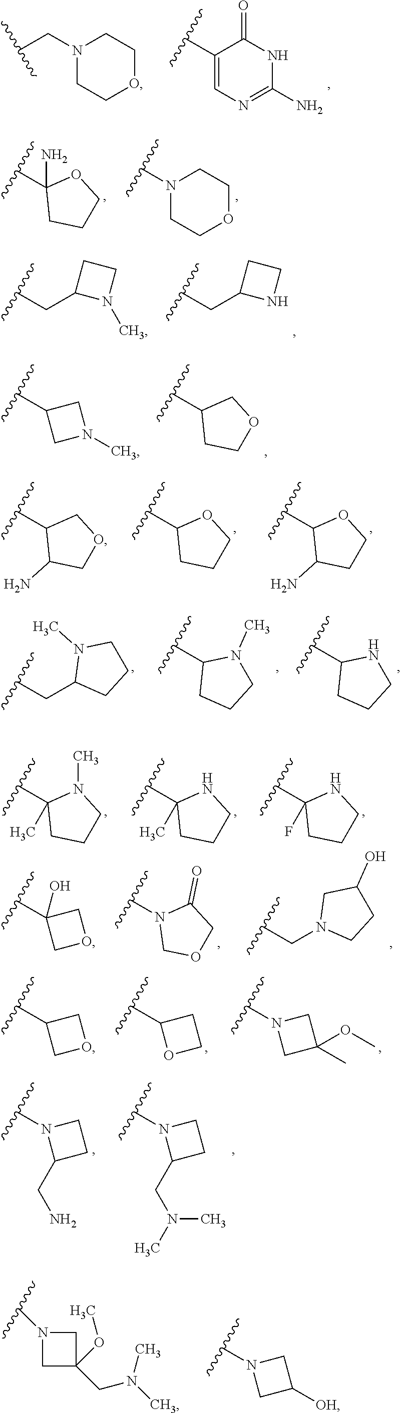

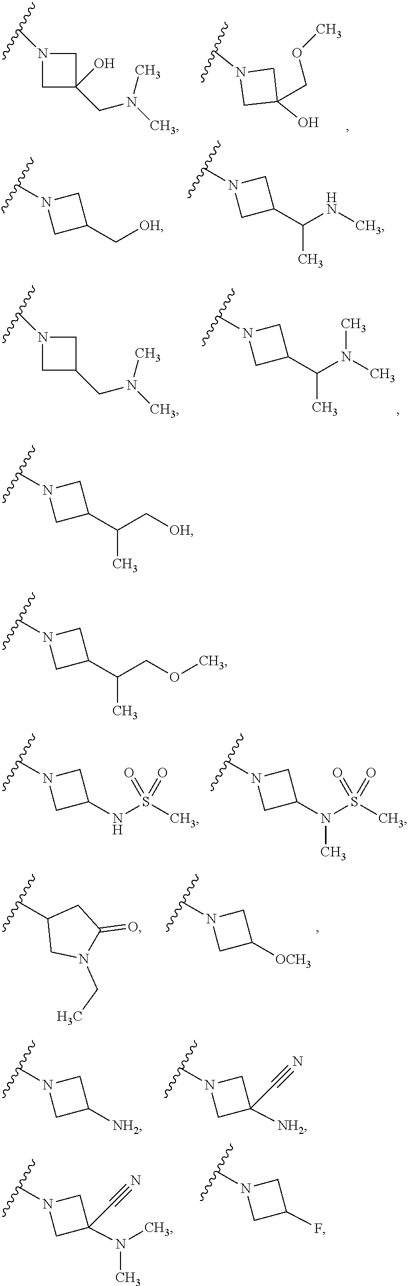

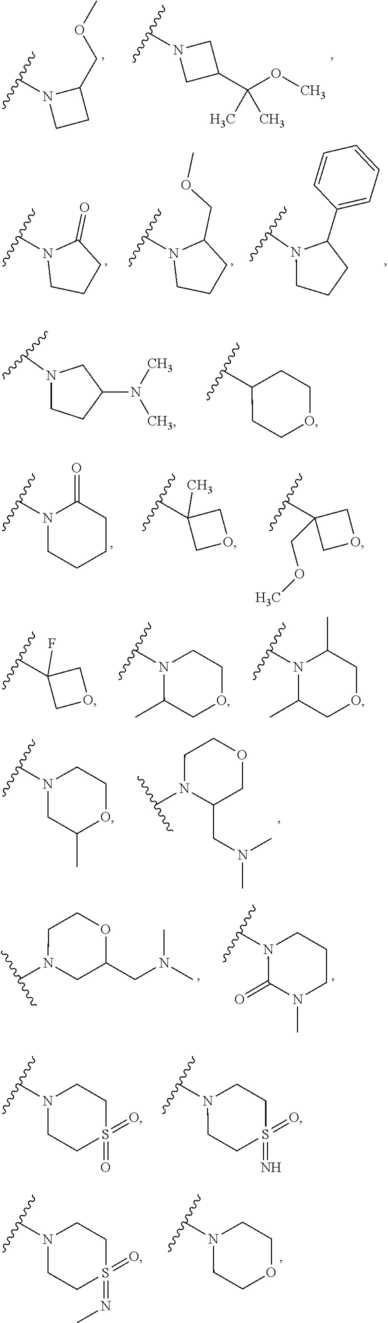

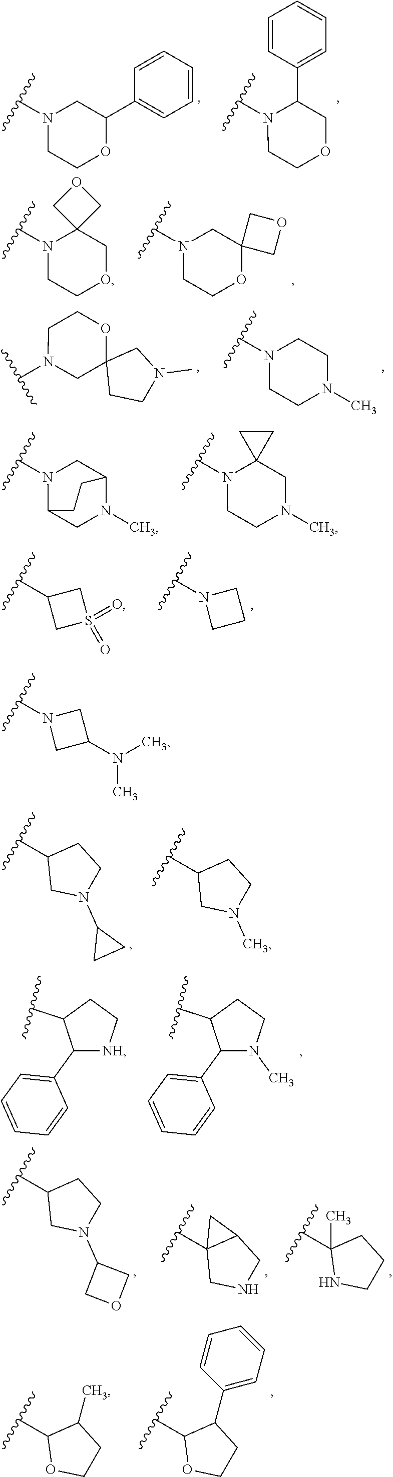

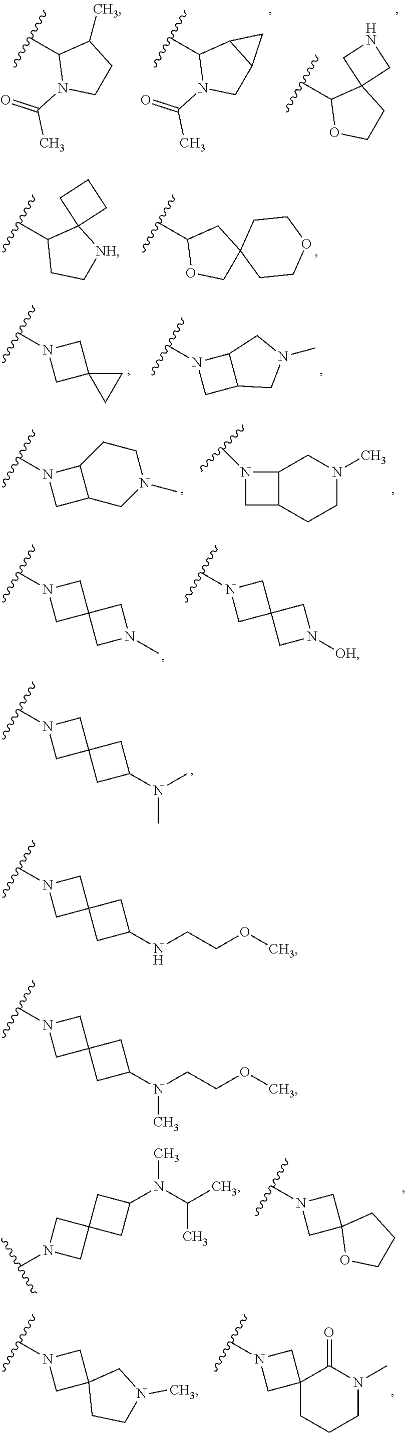

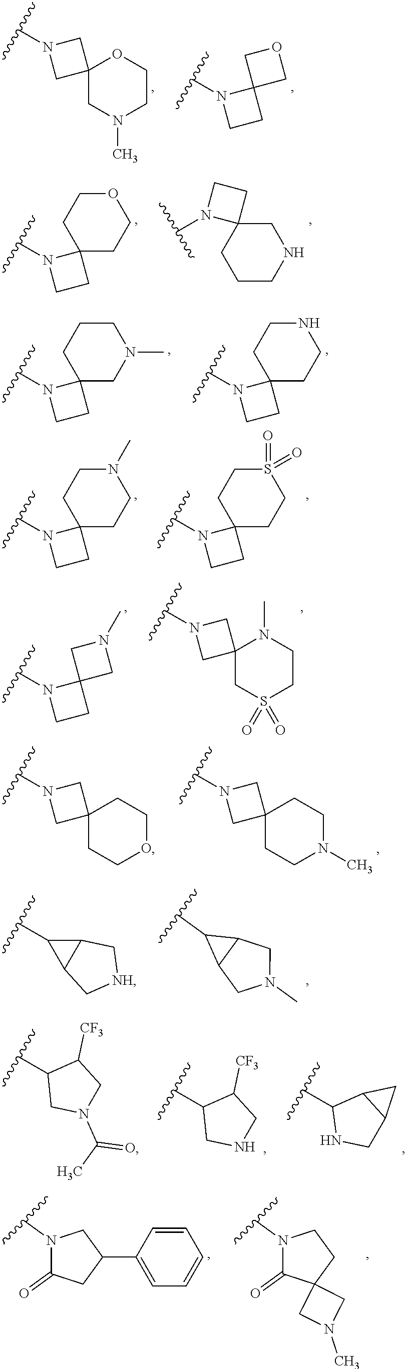

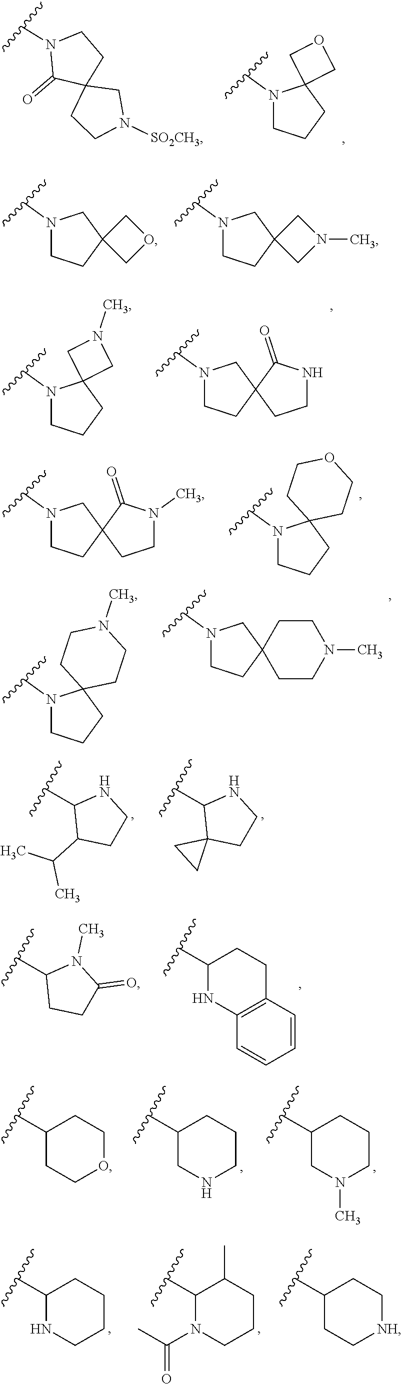

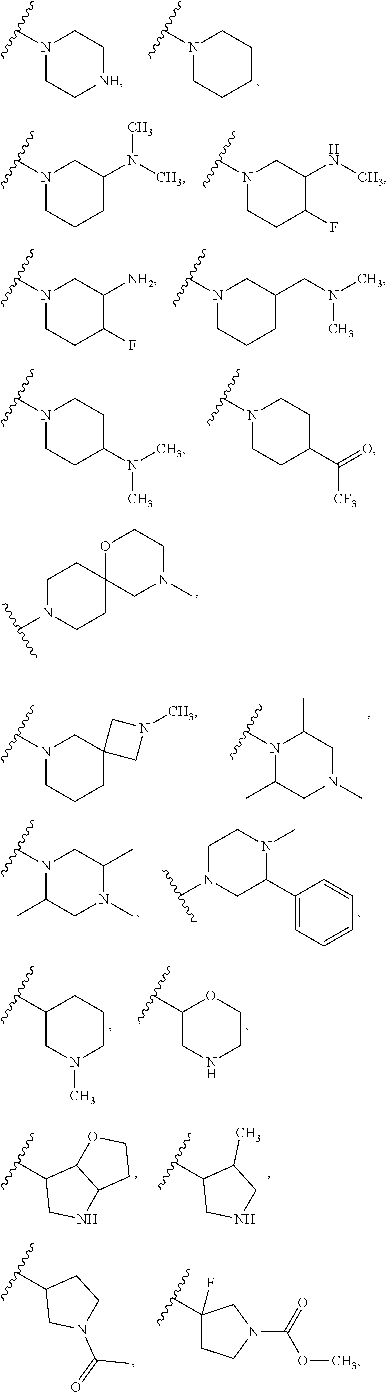

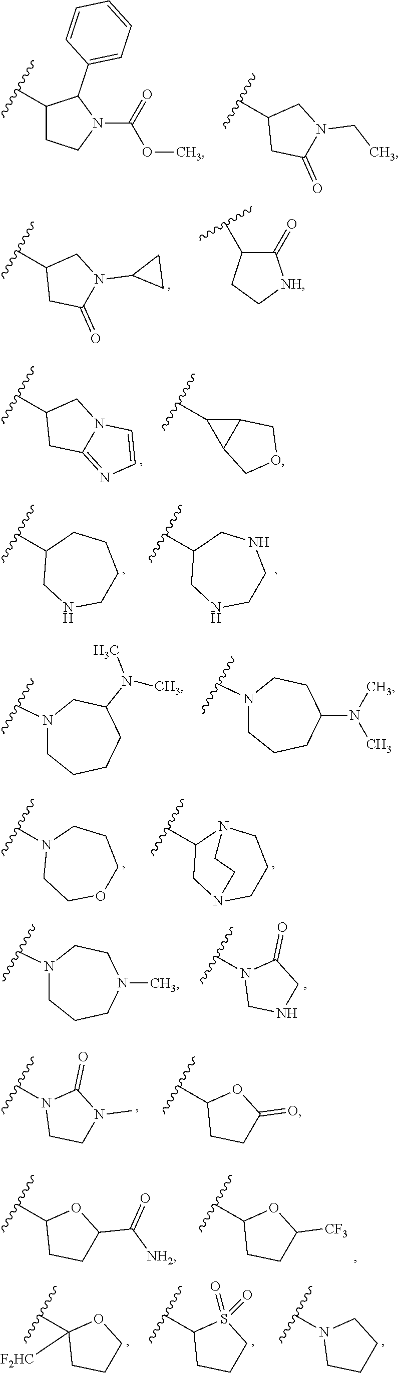

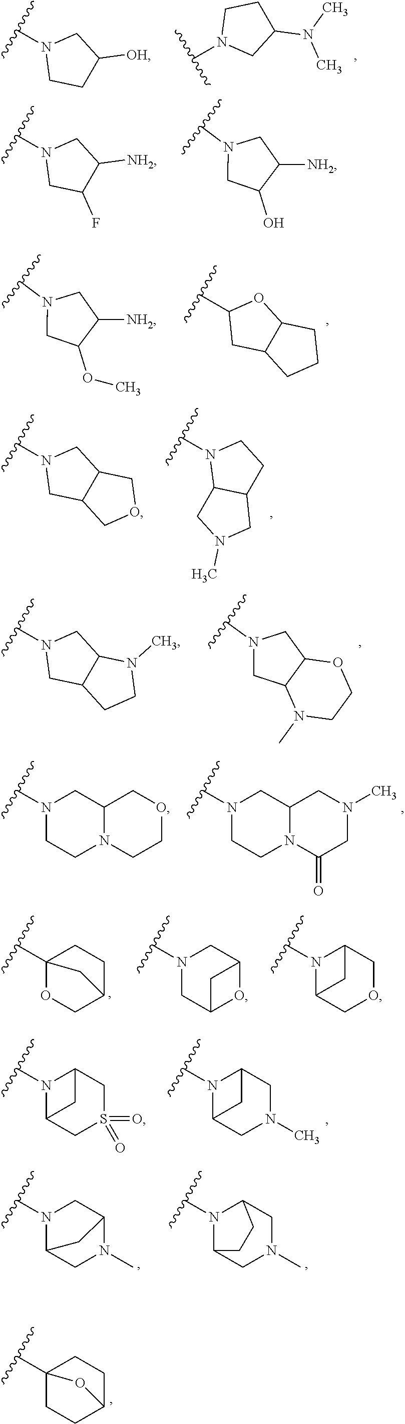

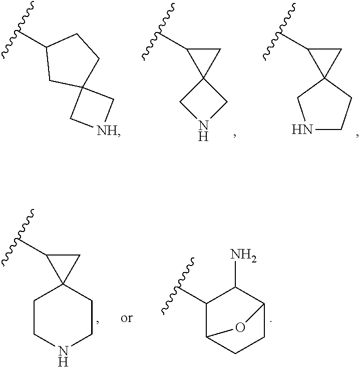 [SMILES: *C1CCC2(CNC2)C1 ; *C1CC12CNC2 ; *C1CC12CCNC2 ; *C1CC12CCNCC2 ; *C1C2CCC(O2)C1N]

[88] The compound, or a pharmaceutically acceptable salt thereof, of any one of paragraphs [1] to [68], wherein W is optionally substituted 3 to 8-membered cycloalkyl.

[89] The compound, or a pharmaceutically acceptable salt thereof, of paragraph [88], wherein W is

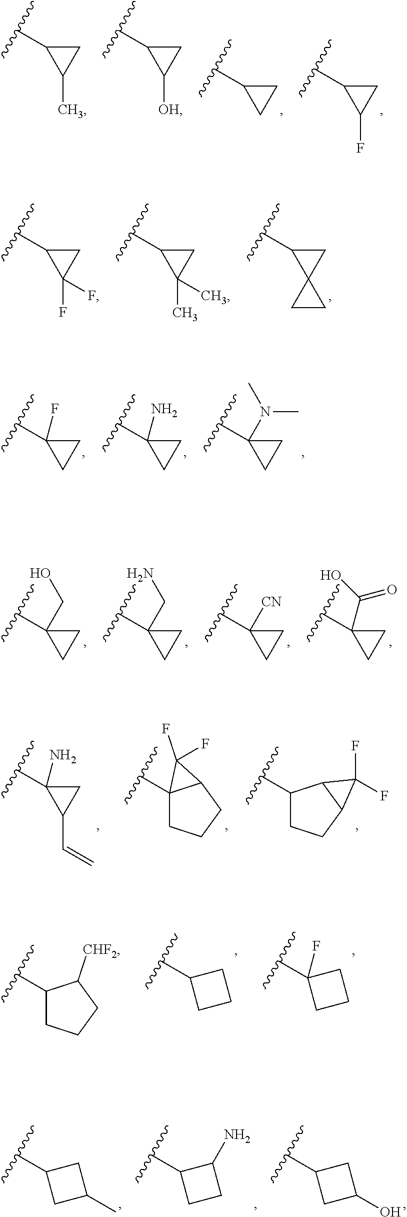

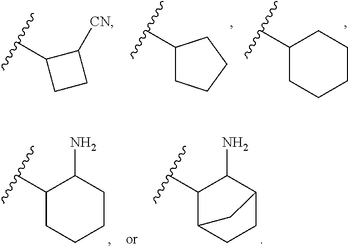 [SMILES: *C1CCC1C#N ; *C1CCCC1 ; *C1CCCCC1 ; *C1CCCCC1N ; *C1C2CCC(C2)C1N]

[90] The compound, or a pharmaceutically acceptable salt thereof, of any one of paragraphs [1] to [68], wherein W is optionally substituted 3 to 8-membered heteroaryl.

[91] The compound, or a pharmaceutically acceptable salt thereof, of paragraph [90], wherein W is

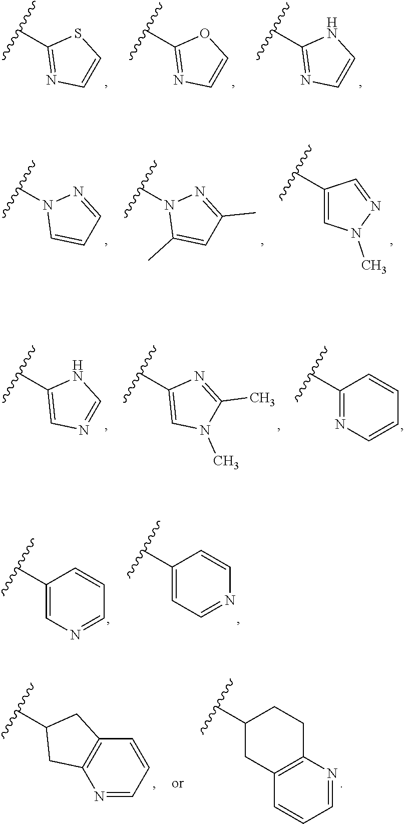

[92] The compound, or a pharmaceutically acceptable salt thereof, of any one of paragraphs [1] to [68], wherein W is optionally substituted 6- to 10-membered aryl.

[93] The compound, or a pharmaceutically acceptable salt thereof, or paragraph [92], wherein W is phenyl, 4-hydroxy-phenyl, or 2,4-methoxy-phenyl.

[94] A compound, or a pharmaceutically acceptable salt thereof, of Table 1 or 2. [95] A pharmaceutical composition comprising a compound, or a pharmaceutically acceptable salt thereof, of any one of paragraphs [1] to [94] and a pharmaceutically acceptable excipient.

[96] A method of treating cancer in a subject in need thereof, the method comprising administering to the subject a therapeutically effective amount of a compound, or a pharmaceutically acceptable salt thereof, of any one of paragraphs [1] to [94] or a pharmaceutical composition of paragraph [95].

[97] The method of paragraph [96], wherein the cancer is pancreatic cancer, colorectal cancer, non-small cell lung cancer, gastric cancer, esophageal cancer, ovarian cancer or uterine cancer.

[98] The method of paragraph [97], wherein the cancer comprises a Ras mutation.

[99] The method of paragraph [98] wherein the Ras mutation is at position 12, 13 or 61.

[100] The method of paragraph [98] wherein the Ras mutation is K-Ras G12C, K-Ras G12D, K-Ras G12V, K-Ras G12S, K-Ras G13C, K-Ras G13D, or K-Ras Q61L.

[101] A method of treating a Ras protein-related disorder in a subject in need thereof, the method comprising administering to the subject a therapeutically effective amount of a compound, or a pharmaceutically acceptable salt thereof, of any one of paragraphs [1] to [94] or a pharmaceutical composition of paragraph [95].

[102] A method of inhibiting a Ras protein in a cell, the method comprising contacting the cell with an effective amount of a compound, or a pharmaceutically acceptable salt thereof, of any one of paragraphs [1] to [94] or a pharmaceutical composition of paragraph [95].

[103] The method of paragraph [101] or [102], wherein the Ras protein is K-Ras G12C, K-Ras G12D, K-Ras G12V, K-Ras G12S, K-Ras G13C, K-Ras G13D, or K-Ras Q61L.

[104] The method of paragraph [102] or [103], wherein the cell is a cancer cell.

[105] The method of paragraph [104], wherein the cancer cell is a pancreatic cancer cell, a colorectal cancer cell, a non-small cell lung cancer cell, a gastric cancer cell, an esophageal cancer cell, an ovarian cancer cell, or a uterine cancer cell.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:

| | |
|---|---|
| CH₂Cl₂, | Methylene chloride, Dichloromethane |
| DCM | |
| CH₃CN, | Acetonitrile |
| MeCN | |
| CuI | Copper (I) iodide |
| DIPEA | Diisopropylethyl amine |
| DMF | N,N-Dimethylformamide |
| EtOAc | Ethyl acetate |
| h | hour |
| H₂O | Water |
| HCl | Hydrochloric acid |
| K₃PO₄ | Potassium phosphate (tribasic) |
| MeOH | Methanol |
| Na₂SO₄ | Sodium sulfate |
| NMP | N-methyl pyrrolidone |
| Pd(dppf)Cl₂ | [1,1′- |
| | Bis(diphenylphosphino)ferrocene]dichloropalladium(II) |

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a ODa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

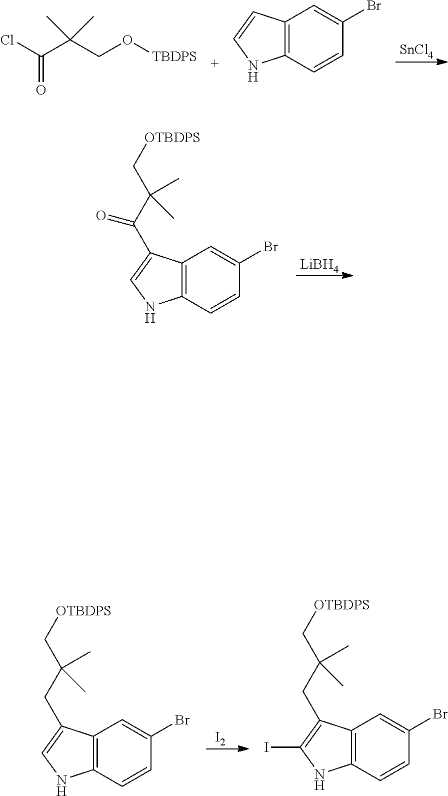

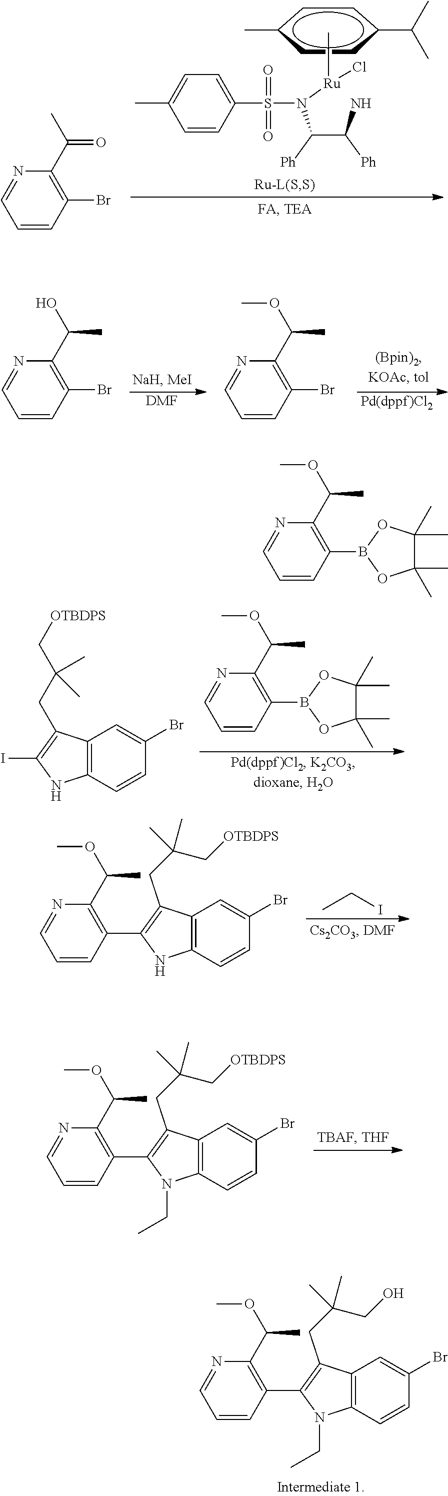

Step 1. To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (100 mL×4), dried over Na₂SO₄ and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z: [M+Na] calc'd for C₂₉H₃₂BrNO₂SiNa 556.1; found 556.3.

Step 2. To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH·H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z: [M+H] calc'd for C₂₉H₃₄BrNOSi 519.2; found 520.1; ¹H NMR (400 MHz, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

Step 3. To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄ and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHz, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

Step 4. To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl)ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give (1S)-1-(3-bromopyridin-2-yl)ethanol (100 g, 74% yield) a an oil. LCMS (ESI): m/z: [M+H] calc'd for C₇H₈BrNO 201.1; found 201.9.

Step 5. To a stirred mixture of (1S)-1-(3-bromopyridin-2-yl)ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI): m/z: [M+H] calc'd for C₈H₁₀BrNO 215.0; found 215.9.

Step 6. To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis(pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl)pyridine (100 g, 63% yield) as a semi-solid. LCMS (ESI): m/z: [M+H] calc'd for C₄H₂₂BNO₃: 263.2; found 264.1.

Step 7. To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl)pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI): m/z: [M+H] calc'd for C₃₇H₄₃BrN₂O₂Si 654.2; found 655.1.

Step 8. To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to rt and stirred for 16 h then H₂O (4 L) added and the mixture extracted with EtOAc (3×1.5 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z: [M+H] calc'd for C₃₉H₄₇BrN₂O₂Si 682.3; found 683.3.

Step 9. To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI): m/z: [M+H] calc'd for C₂₃H₂₉BrN₂O₂ 444.1; found 445.1.

### Intermediate 1. Alternative Synthesis through Fisher Indole Route

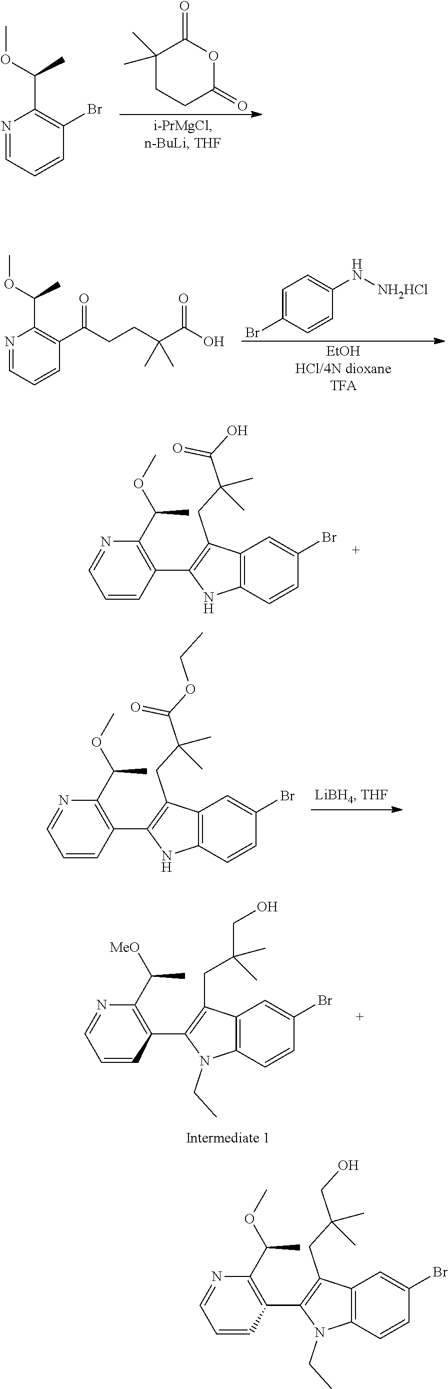

Step 1. To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z: [M+H] calc'd for C₁₅H₂₁NO₄ 279.2; found 280.1.

Step 2. To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl)hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5, concentrated under reduced pressure and the residue adjusted to pH ˜5 with saturated NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.

Step 3. To a mixture of 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. EtI (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI): m/z: [M+H] calc'd for C₂₅H₃₁BrN₂O₃ 486.2; found 487.2.

Step 4. To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl)pyridin-3-yl)-1H-indol-3-yl)-2,2-dimethylpropan-1-ol (as single atropisomers) (60 g, 38% yield) and (40 g, 26% yield) both as solids. LCMS (ESI): m/z: [M+H] calc'd for C₂₃H₂₉BrN₂O₂ 444.1; found 445.2.

### Intermediate 2 and Intermediate 4. Synthesis of (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate

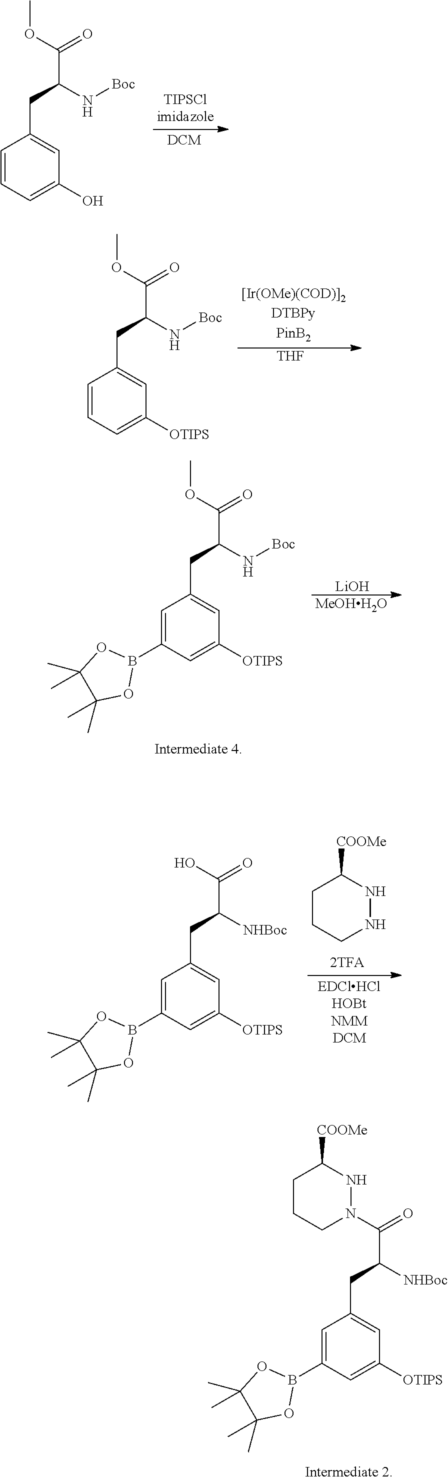

Step 1. To a mixture of (S)-methyl 2-(tert-butoxycarbonylamino)-3-(3-hydroxyphenyl)propanoate (10.0 g, 33.9 mmol) in DCM (100 mL) was added imidazole (4.6 g, 67.8 mmol) and TIPSCI (7.8 g, 40.7 mmol). The mixture was stirred at rt overnight then diluted with DCM (200 mL) and washed with H₂O (150 mL×3). The organic layer was dried over anhydrous Na₂SO₄, filtered, concentrated under reduced pressure and the residue was purified by silica gel column chromatography to give (S)-methyl 2-(tert-butoxycarbonylamino)-3-(3-(triisopropylsilyloxy)phenyl)-propanoate (15.0 g, 98% yield) as an oil. LCMS (ESI): m/z: [M+Na] calc'd for C₂₄H₄₁NO₅SiNa 474.3; found 474.2.

Step 2. A mixture of (S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]₂ (1.1 g, 1.7 mmol) and 4-tert-butyl-2-(4-tert-butyl-2-pyridyl)pyridine (1.3 g, 5.0 mmol) was purged with Ar (×3), then THF (75 mL) was added and the mixture placed under an atmosphere of Ar and sealed. The mixture was heated to 80° C. and stirred for 16 h, concentrated under reduced pressure and the residue was purified by silica gel column chromatography to give (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate (7.5 g, 78% yield) as a solid. LCMS (ESI): m/z: [M+Na] calc'd for C₃₀H₅₂BNO₇SiNa 600.4; found 600.4; ¹H NMR (300 MHz, CD₃OD) δ 7.18 (s, 1H), 7.11 (s, 1H), 6.85 (s, 1H), 4.34 (m, 1H), 3.68 (s, 3H), 3.08 (m, 1H), 2.86 (m, 1H), 1.41-1.20 (m, 26H), 1.20-1.01 (m, 22H), 0.98-0.79 (m, 4H).

Step 3. To a mixture of triisopropylsilyl (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to pH ˜5 with 1M HCl and extracted with EtOAc (250 mL×2). The combined organic layers were washed with brine (100 mL×3), dried over anhydrous Na₂SO₄, filtered and the filtrate concentrated under reduced pressure to give (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (3.7 g, 95% yield), which was used directly in the next step without further purification. LCMS (ESI): m/z: [M+NH₄] calc'd for C₂₉H₅₀BNO₇SiNH₄ 581.4; found 581.4.

Step 4. To a mixture of methyl (S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (24 g, 42.6 mmol) in DCM (50 mL) then HOBt (1.21 g, 9.0 mmol) and EDCI HCl salt (12.9 g, 67.6 mmol). The mixture was warmed to rt and stirred for 16 h, then diluted with DCM (200 mL) and washed with H₂O (3×150 mL). The organic layer was dried over anhydrous Na₂SO, filtered, the filtrate concentrated under reduced pressure and the residue was purified by silica gel column chromatography to give methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (22 g, 71% yield) as an oil. LCMS (ESI): m/z: [M+H] calc'd for C₃₅H₆₀BN₃O₈Si 689.4; found 690.5.

### Intermediate 3. Synthesis of (S)-tert-butyl 3-methyl-2-((S)—N-methylpyrrolidine-3-carboxamido)butanoate

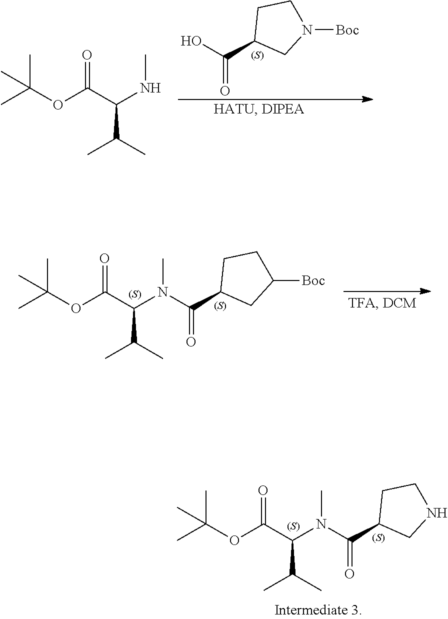

Step 1. To a mixture of (S)-1-(tert-butoxycarbonyl)pyrrolidine-3-carboxylic acid (2.2 g, 10.2 mmol) in DMF (10 mL) at rt was added HATU (7.8 g, 20.4 mmol) and DIPEA (5 mL). After stirring at rt for 10 min, tert-butyl methyl-L-valinate (3.8 g, 20.4 mmol) in DMF (10 mL) was added. The mixture was stirred at rt for 3 h, then diluted with DCM (40 mL) and H₂O (30 mL). The aqueous and organic layers were separated, and the organic layer was washed with H₂O (3×30 mL), brine (30 mL), dried over anhydrous Na₂SO₄ and filtered. The filtrate was concentrated under reduced pressure and the residue was purified by silica gel column chromatography to give (S)-tert-butyl 3-(((S)-1-(tert-butoxy)-3-methyl-1-oxobutan-2-yl)(methyl)carbamoyl)pyrrolidine-1-carboxylate (3.2 g, 82% yield) as an oil. LCMS (ESI): m/z: [M+Na] calc'd for C₂₀H₃₆N₂O₅Na 407.3; found 407.2.

Step 2. A mixture of (S)-tert-butyl 3-(((S)-1-(tert-butoxy)-3-methyl-1-oxobutan-2-yl)(methyl)carbamoyl)pyrrolidine-1-carboxylate (3.2 g, 8.4 mmol) in DCM (13 mL) and TFA (1.05 g, 9.2 mmol) was stirred at rt for 5 h. The mixture was concentrated under reduced pressure to give (S)-tert-butyl 3-methyl-2-((S)—N-methylpyrrolidine-3-carboxamido)butanoate (2.0 g, 84% yield) as an oil. LCMS (ESI): m/z: [M+H] calc'd for C₁₅H₂₈N₂O₃ 284.2; found 285.2.

### Intermediate 5. Synthesis of tert-butyl ((6³S,4S)-11-ethyl-12-(2-((S)-1-methoxyethyl)pyridin-3-yl)-10,10-dimethyl-5,7-dioxo-25-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

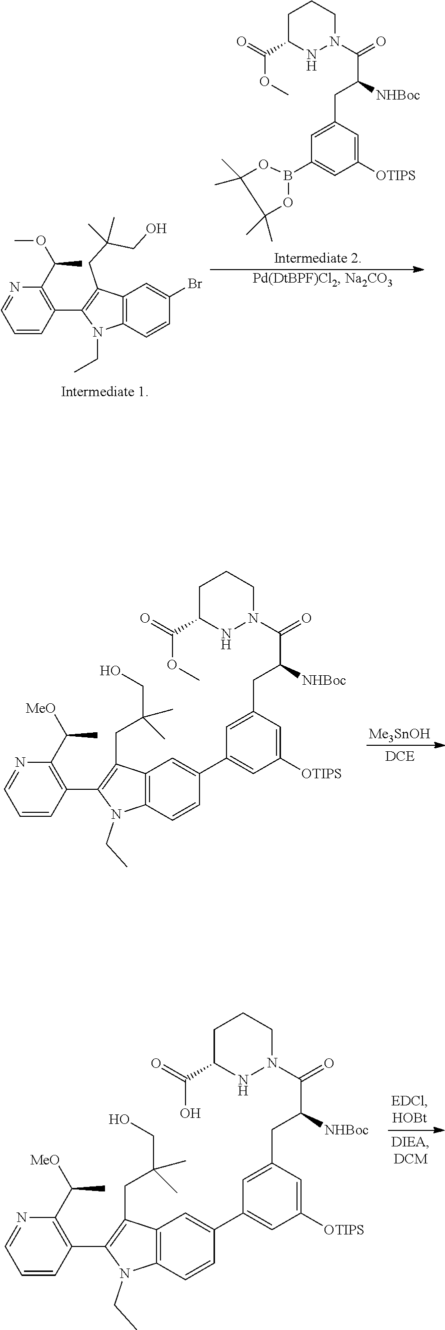

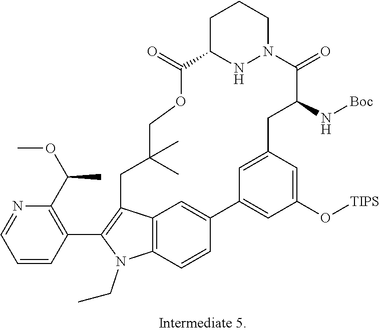

Step 1. To a stirred mixture of 3-(5-bromo-1-ethyl-2-[2-[(1S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in 1,4-dioxane (750 mL) at rt under an atmosphere of Ar was added Na₂CO₃ (17.9 g, 168.4 mmol), Pd(DtBPF)Cl₂ (4.39 g, 6.7 mmol) and H₂O (150.00 mL) in portions. The mixture was heated to 85° C. and stirred for 3 h, cooled, diluted with H₂O (2 L) and extracted with EtOAc (3×1 L). The combined organic layers were washed with brine (2×500 mL), dried over anhydrous Na₂SO₄ and filtered. The filtrate was concentrated under reduced pressure and the residue was purified by silica gel column chromatography to give methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 72% yield) as a solid. LCMS (ESI): m/z: [M+H] calc'd for C₅₂H₇₇N₅O₈Si 927.6; found 928.8.

Step 2. To a stirred mixture of methyl (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at rt was added trimethyltin hydroxide (48.7 g, 269 mmol) in portion. The mixture was heated to 65° C. and stirred for 16 h, then filtered and the filter cake washed with DCM (3×150 mL). The filtrate was concentrated under reduced pressure to give (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g, crude), which was used directly in the next step without further purification. LCMS (ESI): m/z: [M+H] calc'd for C₅₁H₇₅N₅O₈Si 913.5; found 914.6.

Step 3. To a stirred mixture of (3S)-1-[(2S)-2-[(tert-butoxycarbonyl)amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (297 g, 2.3 mol), HOBT (51.7 g, 383 mmol) and EDCI (411 g, 2.1 mol) in portions. The mixture was warmed to rt and stirred for 16 h, then diluted with DCM (1 L), washed with brine (3×1 L), dried over anhydrous Na₂SO₄ and filtered. The filtrate was concentrated under reduced pressure and the residue was purified by silica gel column chromatography to give tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (36 g, 42% yield) as a solid. LCMS (ESI): m/z: [M+H] calc'd for C₅₁H₇₃N₅O₇Si 895.5; found 896.5.

### Intermediate 6. Synthesis of tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate

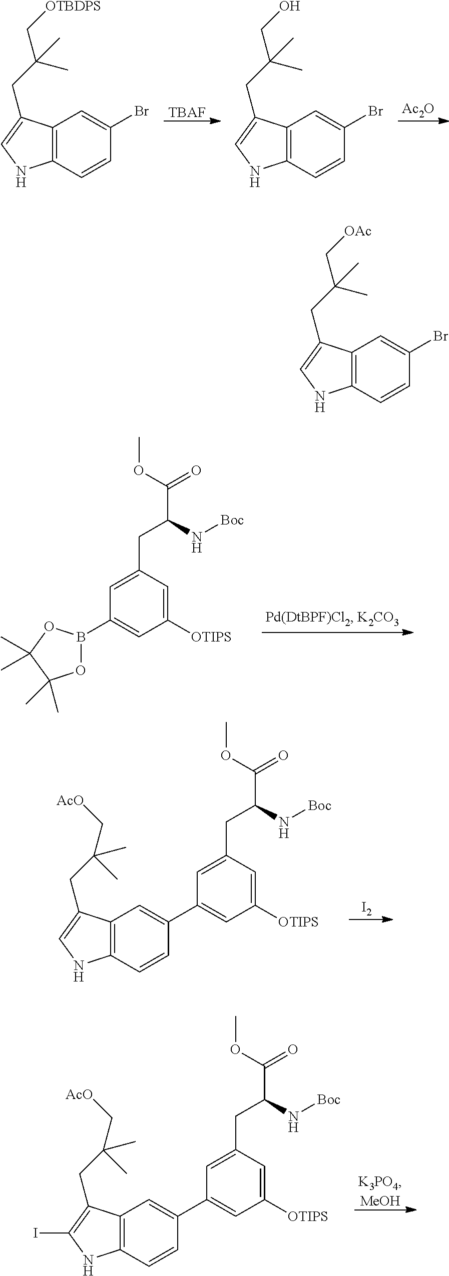

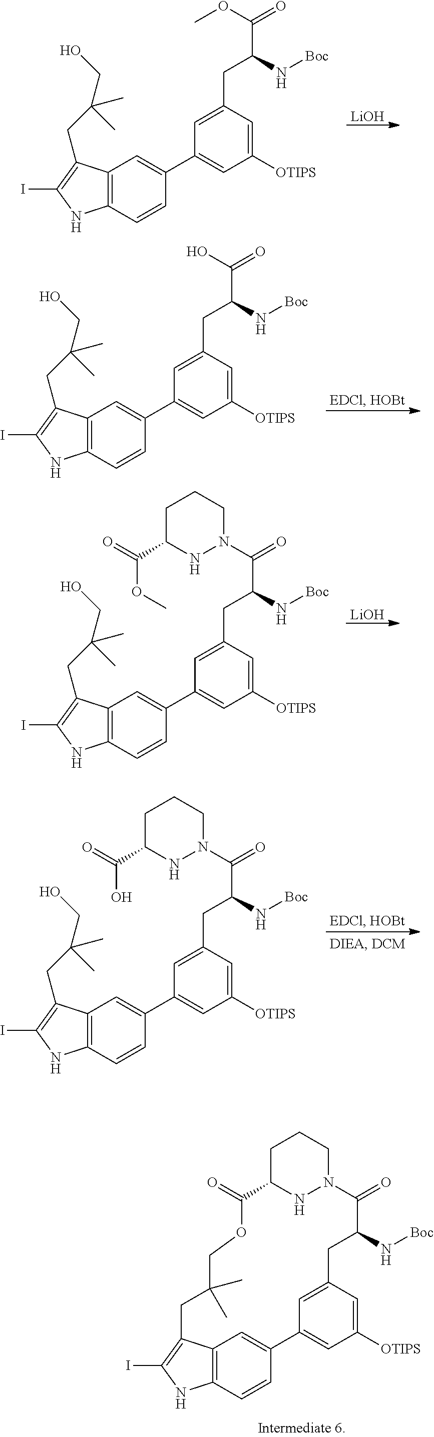

Step 1. This reaction was undertaken on 5-batches in parallel on the scale illustrated below.

Into a 2 L round-bottom flasks each were added 5-bromo-3-[3-[(tert-butyldiphenylsilyl)oxy]-2,2-dimethylpropyl]-1H-indole (100 g, 192 mmol) and TBAF (301.4 g, 1.15 mol) in THF (1.15 L) at rt. The resulting mixture was heated to 50° C. and stirred for 16 h, then the mixture was concentrated under reduced pressure. The combined residues were diluted with H₂O (5 L) and extracted with EtOAc (3×2 L). The combined organic layers were washed with brine (2×1.5 L), dried over anhydrous Na₂SO₄ and filtered. The filtrate was concentrated under reduced pressure and the residue was purified by silica gel column chromatography to give 3-(5-bromo-1H-indol-3-yl)-2,2-dimethylpropan-1-ol (310 g, crude) as a solid. LCMS (ESI): m/z: [M+H] calc'd for C₁₃H₁₆BrNO 281.0 and 283.0; found 282.1 and 284.1.

Step 2. This reaction was undertaken on 2-batches in parallel on the scale illustrated below.

To a stirred mixture of 3-(5-bromo-1H-indol-3-yl)-2,2-dimethylpropan-1-ol (135 g, 478 mmol) and TEA (145.2 g, 1.44 mol) in DCM (1.3 L) at 0° C. under an atmosphere of N₂ was added Ac₂O (73.3 g, 718 mmol) and DMAP (4.68 g, 38.3 mmol) in portions. The resulting mixture was stirred for 10 min at 0° C., then washed with H₂O (3×2 L). The organic layers from each experiment were combined and washed with brine (2×1 L), dried over anhydrous Na₂SO₄ and filtered. The filtrate was concentrated under reduced pressure and the residue was purified by column chromatography to give 3-(5-bromo-1H-indol-3-yl)-2,2-dimethylpropyl acetate (304 g, 88% yield) as a solid. ¹H NMR (400 MHz, DMSO-d₆) δ 11.16-11.11 (m, 1H), 7.69 (d, J=2.0 Hz, 1H), 7.32 (d, J=8.6 Hz, 1H), 7.19-7.12 (m, 2H), 3.69 (s, 2H), 2.64 (s, 2H), 2.09 (s, 3H), 0.90 (s, 6H).

Step 3. This reaction was undertaken on 4-batches in parallel on the scale illustrated below.

Into a 2 L round-bottom flasks were added methyl (2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoate (125 g, 216 mmol), 1,4-dioxane (1 L), H₂O (200 mL), 3-(5-bromo-1H-indol-3-yl)-2,2-dimethylpropyl acetate (73.7 g, 227 mmol), K₂CO₃ (59.8 g, 433 mmol) and Pd(DtBPF)Cl₂ (7.05 g, 10.8 mmol) at rt under an atmosphere of Ar. The resulting mixture was heated to 65° C. and stirred for 2 h, then diluted with H₂O (10 L) and extracted with EtOAc (3×3 L). The combined organic layers were washed with brine (2×2 L), dried over anhydrous Na₂SO₄ and filtered. The filtrate was concentrated under reduced pressure and the residue was purified by column chromatography to give methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate (500 g, 74% yield) as an oil. LCMS (ESI): m/z: [M+Na] calc'd for C₃₉H₅₈N₂O₇SiNa 717.4; found 717.3.

Step 4. This reaction was undertaken on 3-batchs' in parallel on the scale illustrated below.

To a stirred mixture of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl)amino]propanoate (150 g, 216 mmol) and NaHCO₃ (21.76 g, 259 mmol) in THF (1.5 L) was added AgOTf (66.5 g, 259 mmol) in THF dropwise at 0° C. under an atmosphere of nitrogen. 12 (49.3 g, 194 mmol) in THF was added dropwise over 1 h at 0° C. and the resulting mixture was stirred for an additional 10 min at 0° C. The combined experiments were diluted with aqueous Na₂S₂O₃ (5 L) and extracted with EtOAc (3×3 L). The combined organic layers were washed with brine (2×1.5 L), dried over anhydrous Na₂SO₄ and filtered. The filtrate was concentrated under reduced pressure and the residue was purified by column chromatography to give methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate (420 g, 71% yield) as an oil. LCMS (ESI): m/z: [M+Na] calc'd for C₃₉H₅₇IN₂O₇SiNa, 843.3; found 842.9.

Step 5. This reaction was undertaken on 3-batches in parallel on the scale illustrated below.

To a 2 L round-bottom flask were added methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl)amino]propanoate (140 g, 171 mmol), MeOH (1.4 L) and K₃PO₄ (108.6 g, 512 mmol) at 0° C. The mixture was warmed to rt and stirred for 1 h, then the combined experiments were diluted with H₂O (9 L) and extracted with EtOAc (3×3 L). The combined organic layers were washed with brine (2×2 L), dried over anhydrous Na₂SO₄, filtered and the filtrate was concentrated under reduced pressure to give methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate (438 g, crude) as a solid. LCMS (ESI): m/z: [M+Na] calc'd for C₃₇H₅₅IN₂O₆SiNa 801.3; found 801.6.

Step 6. This reaction was undertaken on 3-batches in parallel on the scale illustrated below.

To a stirred mixture of methyl (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoate (146 g, 188 mmol) in THF (1.46 L) was added LiOH (22.45 g, 937 mmol) in H₂O (937 mL) dropwise at 0° C. The resulting mixture was warmed to rt and stirred for 1.5 h [note: LCMS showed 15% de-TIPS product]. The mixture was acidified to pH 5 with 1M HCl (1M) and the combined experiments were extracted with EtOAc (3×3 L). The combined organic layers were washed with brine (2×2 L), dried over anhydrous Na₂SO₄, filtered and the filtrate was concentrated under reduced pressure to give (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid (402 g, crude) as a solid. LCMS (ESI): m/z: [M+Na] calc'd for C₃₅H₅₃IN₂O₆SiNa 787.3; found 787.6.

Step 7. To a stirred mixture of (2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoic acid (340 g, 445 mmol) and methyl (3S)-1,2-diazinane-3-carboxylate (96.1 g, 667 mmol) in DCM (3.5 L) was added NMM (225 g, 2.2 mol), EDCI (170 g, 889 mmol), HOBT (12.0 g, 88.9 mmol) portionwise at 0° C. The mixture was warmed to rt and stirred for 16 h, then washed with H₂O (3×2.5 L), brine (2×1 L), dried over anhydrous Na₂SO₄ and filtered. The filtrate was concentrated under reduced pressure and the residue was purified by column chromatography to give methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (310 g, 62% yield) as an oil. LCMS (ESI): m/z: [M+H] calc'd for C₄₂H₆₃IN₄O₇Si 890.4; found 890.8.

Step 8. This reaction was undertaken on 3-batches in parallel on the scale illustrated below.

To a stirred mixture of methyl (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (85.0 g, 95.4 mmol) in THF (850 mL) each added LiOH (6.85 g, 286 mmol) in H₂O (410 mL) dropwise at 0° C. under an atmosphere of N₂. The mixture was stirred at 0° C. for 1.5 h [note: LCMS showed 15% de-TIPS product], then acidified to pH 5 with 1M HCl and the combined experiments extracted with EtOAc (3×2 L). The combined organic layers were washed with brine (2×1.5 L), dried over anhydrous Na₂SO₄, filtered and the filtrate was concentrated under reduced pressure to give (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (240 g, crude) as a solid. LCMS (ESI): m/z: [M+H] calc'd for C₄₁H₆₁IN₄O₇Si 876.3; found 877.6.

Step 9. This reaction was undertaken on 2-batches in parallel on the scale illustrated below.

To a stirred mixture of (3S)-1-[(2S)-2-[(tert-butoxycarbonyl)amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (120 g, 137 mmol) in DCM (6 L) was added DIPEA (265 g, 2.05 mol), EDCI (394 g, 2.05 mol), HOBT (37 g, 274 mmol) in portions at 0° C. under an atmosphere of N₂. The mixture was warmed to rt and stirred overnight, then the combined experiments were washed with H₂O (3×6 L), brine (2×6 L), dried over anhydrous Na₂SO₄ and filtered. After filtration, the filtrate was concentrated under reduced pressure. The filtrate was concentrated under reduced pressure and the residue was purified by column chromatography to give tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (140 g, 50% yield) as a solid. LCMS (ESI): m/z [M+H] calc'd for C₄₁H₅₉IN₄O₆Si 858.9; found 858.3.

### Intermediate 7. Synthesis of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

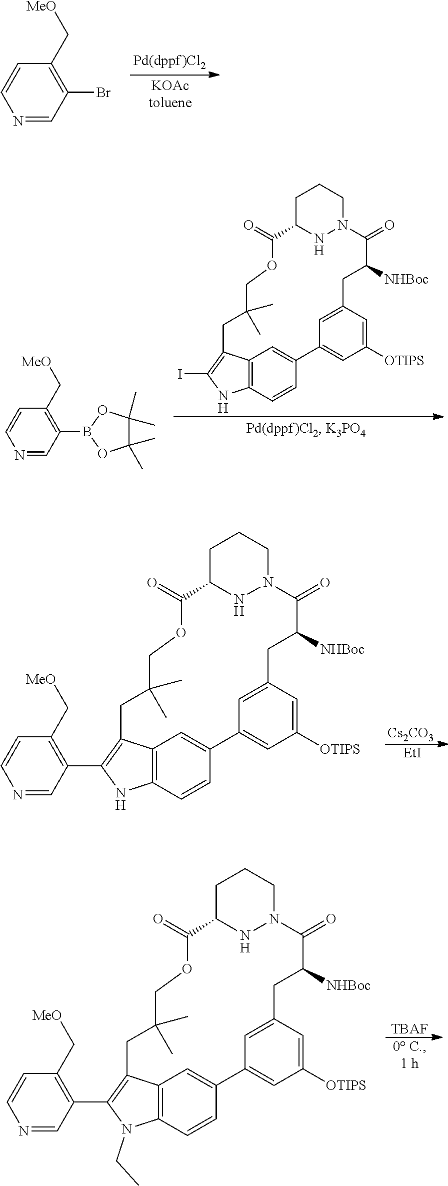

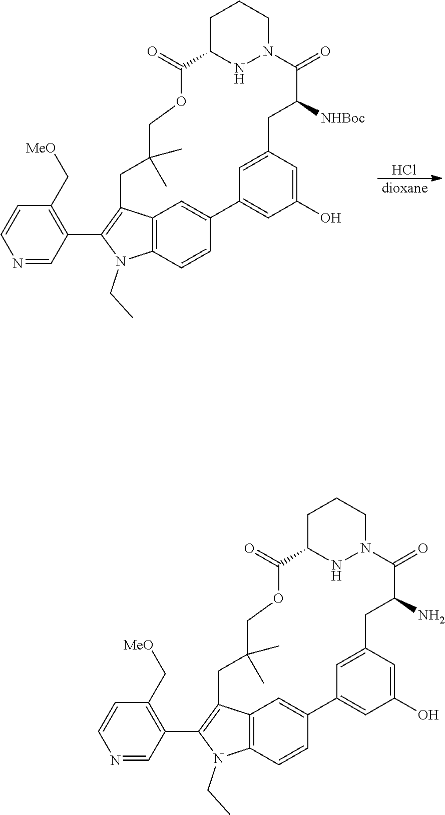

Step 1. To a mixture of 3-bromo-4-(methoxymethyl)pyridine (1.00 g, 5.0 mmol), 4,4,5,5-tetramethyl-2-(tetramethyl-1,3,2-dioxaborolan-2-yl)-1,3,2-dioxaborolane (1.51 g, 5.9 mmol) and KOAc (1.21 g, 12.3 mmol) in toluene (10 mL) at rt under an atmosphere of Ar was added Pd(dppf)Cl₂ (362 mg, 0.5 mmol). The mixture was heated to 110° C. and stirred overnight, then concentrated under reduced pressure to give 4-(methoxymethyl)-3-(4,4,5,5-tetramethyl-1,3,2-dioxaborolan-2-yl)pyridine, which was used directly in the next step directly without further purification. LCMS (ESI): m/z: [M+H] calc'd for C₁₃H₂₂BNO₃ 249.2; found 250.3.

Step 2. To a mixture of 4-(methoxymethyl)-3-(4,4,5,5-tetramethyl-1,3,2-dioxaborolan-2-yl)pyridine (290 mg, 1.16 mmol), K₃PO₄ (371 mg, 1.75 mmol) and tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (500 mg, 0.58 mmol) in 1,4-dioxane (5 mL) and H₂O (1 mL) at rt under an atmosphere of Ar was added Pd(dppf)Cl₂ (43 mg, 0.06 mmol). The mixture was heated to 70° C. and stirred for 2 h, then H₂O added and the mixture extracted with EtOAc (2×10 mL). The combined organic layers were washed with brine (10 mL), dried over anhydrous Na₂SO₄ and filtered. The filtrate was concentrated under reduced pressure and the residue was purified by silica gel column chromatography to give tert-butyl N-[(8S,14S)-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (370 mg, 74% yield) as a foam. LCMS (ESI): m/z: [M+H] calc'd for C₄₈H₆₇N₅O₇Si 853.6; found 854.6.

Step 3. A mixture of tert-butyl N-[(8S,14S)-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (350 mg, 0.41 mmol), Cs₂CO₃ (267 mg, 0.82 mmol) and EtI (128 mg, 0.82 mmol) in DMF (4 mL) was stirred at 35° C. overnight. H₂O was added and the mixture was extracted with EtOAc (2×15 mL). The combined organic layers were washed with brine (15 mL), dried over anhydrous Na₂SO₄ and filtered. The filtrate was concentrated under reduced pressure and the residue was purified by silica gel column chromatography to give tert-butyl N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (350 mg, 97% yield) as an oil. LCMS (ESI): m/z: [M+H] calc'd for C₅₀H₇₁N₅O₇Si 881.5; found 882.6.

Step 4. A mixture of tert-butyl N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (350 mg, 0.4 mmol) and 1M TBAF in THF (0.48 mL, 0.480 mmol) in THF (3 mL) at 0° C. under an atmosphere of Ar was stirred for 1 h. The mixture was concentrated under reduced pressure and the residue was purified by silica gel column chromatography to give tert-butyl N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (230 mg, 80% yield) as an oil. LCMS (ESI): m/z: [M+H] calc'd for C₄₁H₅₁N₅O₇ 725.4; found 726.6.

Step 5. To a mixture of tert-butyl N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (200 mg, 0.28 mmol) in 1,4-dioxane (2 mL) at 0° C. under an atmosphere of Ar was added 4M HCl in 1,4-dioxane (2 mL, 8 mmol). The mixture was allowed to warm to rt and was stirred overnight, then concentrated under reduced pressure to give (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (200 mg). LCMS (ESI): m/z: [M+H] calc'd for C₃₆H₄₃N₅O₅ 625.3; found 626.5.

### Intermediate 8. Synthesis of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

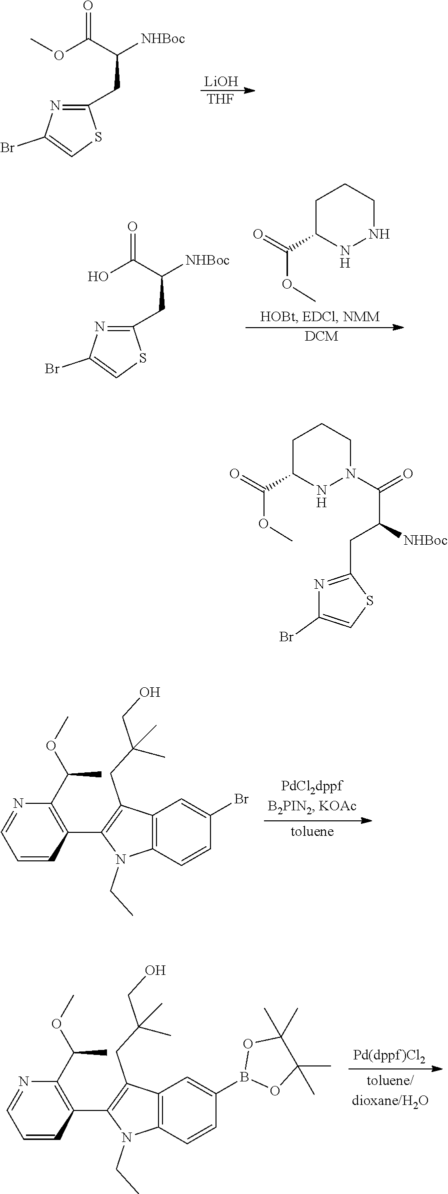

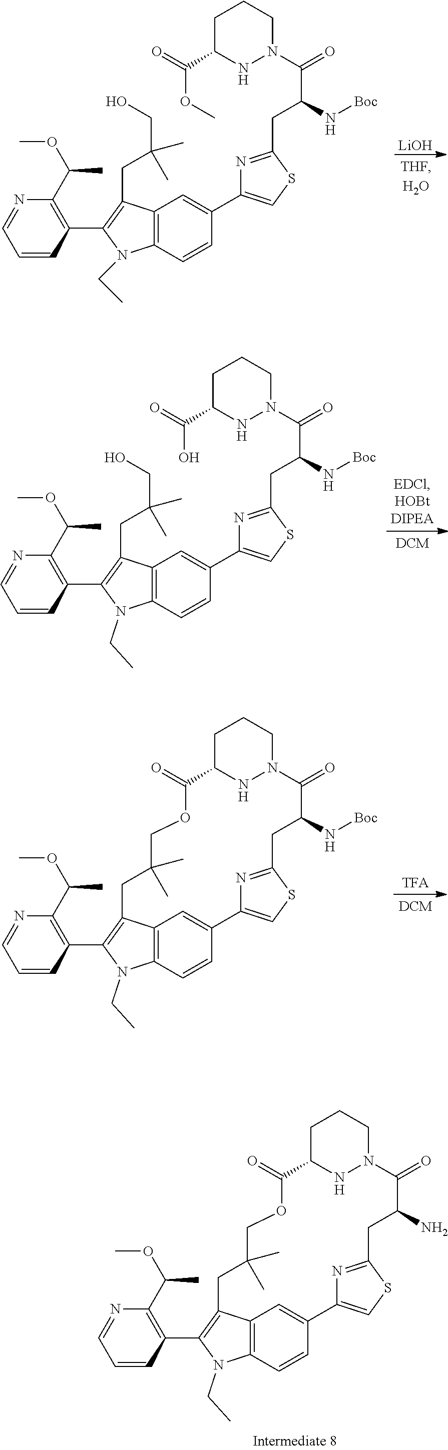

Step 1. To a solution of methyl (2S)-3-(4-bromo-1,3-thiazol-2-yl)-2-[(tert-butoxycarbonyl)amino]propanoate (110 g, 301.2 mmol) in THF (500 mL) and H₂O (200 mL) at room temperature was added LiOH (21.64 g, 903.6 mmol). The solution was stirred for 1 h and was then concentrated under reduced pressure. The residue was adjusted to pH 6 with 1 M HCl and then extracted with DCM (3×500 mL). The combined organic layers were, dried over Na₂SO₄, filtered, and concentrated under reduced pressure to give (S)-3-(4-bromothiazol-2-yl)-2-((tert-butoxycarbonyl)amino)propanoic acid (108 g, crude). LCMS (ESI): m/z: [M+H] calc'd for C₁₁H₁₆BrN₂O₄S 351.0; found 351.0.

Step 2. To a solution of (S)-3-(4-bromothiazol-2-yl)-2-((tert-butoxycarbonyl)amino)propanoic acid (70 g, 199.3 mmol) in DCM (500 mL) at 0° C. was added methyl (3S)-1,2-diazinane-3-carboxylate bis(trifluoroacetic acid) salt (111.28 g, 298.96 mmol), NMM (219.12 mL. 1993.0 mmol), EDCI (76.41 g, 398.6 mmol) and HOBt (5.39 g, 39.89 mmol). The solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel column chromatography to give methyl (S)-1-((S)-3-(4-bromothiazol-2-yl)-2-((tert-butoxycarbonyl)amino)propanoyl)hexahydropyridazine-3-carboxylate (88.1 g, 93% yield). LCMS (ESI): m/z: [M+H] calc'd for C₁₇H₂₆BrN₄O₅S 477.1; found 477.1.

Step 3. To a solution of 3-(5-bromo-1-ethyl-2-(2-((S)-1-methoxyethyl)pyridin-3-yl)-1H-indol-3-yl)-2,2-dimethylpropan-1-ol (60 g, 134.7 mmol) in toluene (500 mL) at room temperature was added bis(pinacolato)diboron (51.31 g, 202.1 mmol), Pd(dppf)Cl₂ (9.86 g, 13.4 mmol), and KOAc (26.44 g, 269 mmol). The reaction mixture was then heated to 90° C. and stirred for 2 h. The reaction solution was then cooled to room temperature and concentrated under reduced pressure. The residue was purified by silica gel column chromatography to give (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (60.6 g, 94% yield). LCMS (ESI): m/z [M+H] calc'd for C₂₉H₄₂BN₂O₄ 493.32; found 493.3.

Step 4. To a solution of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl (S)-1-((S)-3-(4-bromothiazol-2-yl)-2-((tert-butoxycarbonyl)amino)propanoyl)hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The mixture was extracted with EtOAc and the combined organic layers were dried over Na₂SO₄, filtered, and concentrated under reduced pressure. The residue was purified by silica gel column chromatography to give methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate (39.7 g, 85% yield). LCMS (ESI): m/z: [M+H] calc'd for C₄₀H₅₅N₆O₇S 763.4; found 763.3.

Step 5. To a solution of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate (39.7 g, 52.0 mmol) in THF (400 mL) and H₂O (100 mL) at room temperature was added LiOH·H₂O (3.74 g, 156.2 mmol). The mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to give (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid (37.9 g, crude). LCMS (ESI): m/z: [M+H] calc'd for C₃₉H₅₃N₆O₇S 749.4; found 749.4.

Step 6. To a solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCI (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel column chromatography to give tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (30 g, 81% yield). LCMS (ESI): m/z: [M+H] calc'd for C₃₉H₅₁N₆O₆S 731.4; found 731.3.

Step 7. To a solution of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate (6 g, 8.21 mmol) in DCM (60 mL) at 0° C. was added TFA (30 mL). The mixture was stirred for 1 h and was then concentrated under reduced pressure to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (7.0 g, crude). LCMS (ESI): m/z: [M+H] calc'd for C₃₄H₄₂N₆O₄S 631.3; found: 630.3.

### Intermediate 9. Synthesis of (S)-3-bromo-5-iodo-2-(1-methoxyethyl) pyridine

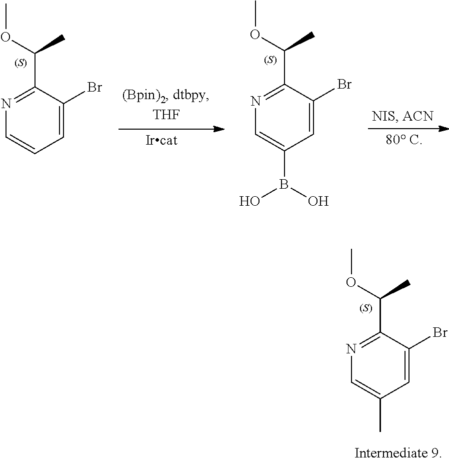

Step 1. To a stirred solution of 3-bromo-2-[(1S)-1-methoxyethyl]pyridine (80.00 g, 370.24 mmol, 1.00 equiv) and bis(pinacolato)diboron (141.03 g, 555.3 mmol, 1.50 equiv) in THF (320 mL) was added dtbpy (14.91 g, 55.5 mmol) and chloro(1,5-cyclooctadiene)iridium(I) dimer (7.46 g, 11.1 mmol) under argon atmosphere. The resulting mixture was stirred for 16 h at 75° C. under argon atmosphere. The mixture was concentrated under reduced pressure. The resulting mixture was dissolved in EtOAc (200 mL) and the mixture was adjusted to pH 10 with Na₂CO₃ (40 g) and NaOH (10 g) (mass 4:1) in water (600 mL). The aqueous layer was extracted with EtOAc (800 mL). The aqueous phase was acidified to pH=6 with HCl (6 N) to precipitate the desired solid to afford 5-bromo-6-[(1S)-1-methoxyethyl]pyridin-3-ylboronic acid (50 g, 52.0% yield) as a light-yellow solid. LCMS (ESI): m/z: [M+H] calc'd for C₈H₁₁BBrNO₃ 259.0; found 260.0.

Step 2. To a stirred solution of 5-bromo-6-[(1S)-1-methoxyethyl]pyridin-3-ylboronic acid (23.00 g, 88.5 mmol) in ACN (230 mL) were added NIS (49.78 g, 221.2 mmol) at room temperature under argon atmosphere. The resulting mixture was stirred for overnight at 80° C. under argon atmosphere. The resulting mixture was concentrated under reduced pressure. The resulting mixture was dissolved in DCM (2.1 L) and washed with Na₂S₂O₃ (3×500 mL). The organic layer was dried over anhydrous Na₂SO₄. After filtration, the filtrate was concentrated under reduced pressure. The residue was purified by silica gel column chromatography to afford (S)-3-bromo-5-iodo-2-(1-methoxyethyl)pyridine (20 g, 66.0% yield). LCMS (ESI): m/z: [M+H] calc'd for C₈H₉BrINO 340.9; found 341.7.

### Intermediate 10. Synthesis of tert-butyl ((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

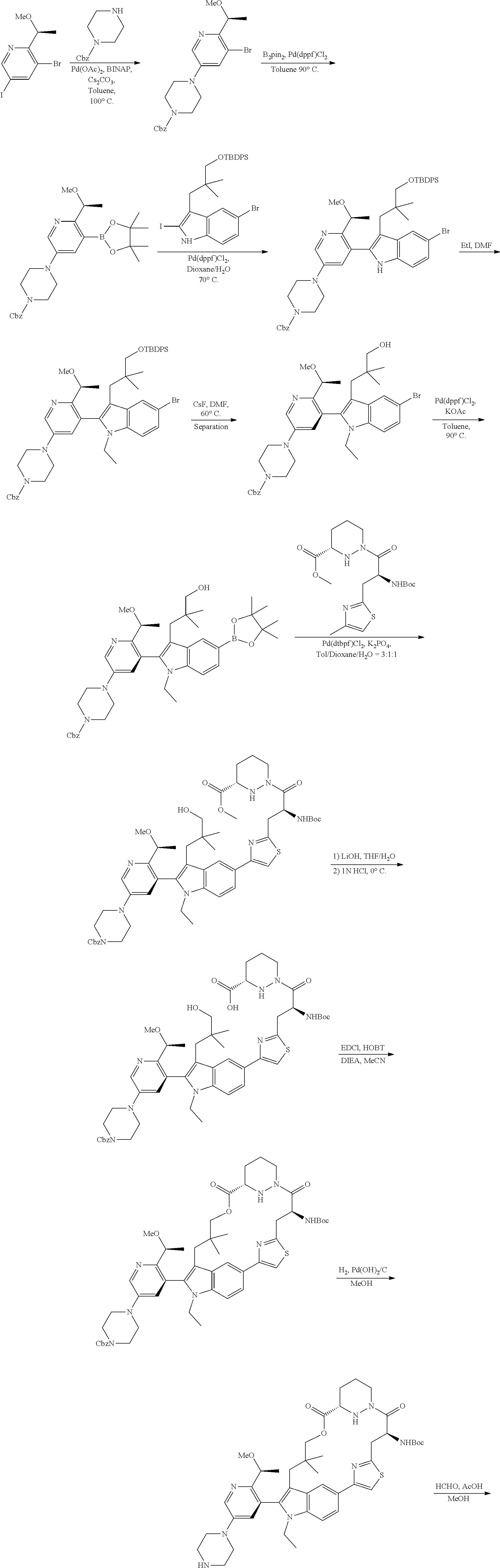

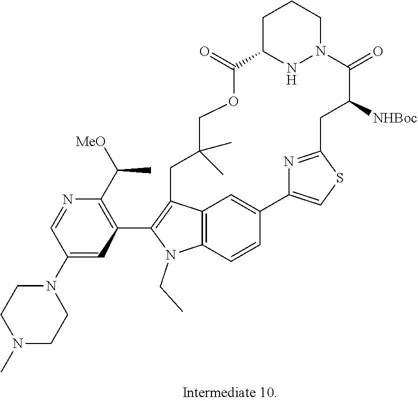

Step 1. Into a 3 L 3-necked round-bottom flask purged and maintained with an inert atmosphere of argon, was placed 3-bromo-5-iodo-2-[(1S)-1-methoxyethyl]pyridine (147 g, 429.8 mmol) benzyl piperazine-1-carboxylate (94.69 g, 429.8 mmol), Pd(OAC)₂ (4.83 g, 21.4 mmol), BINAP (5.35 g, 8.6 mmol), Cs₂CO₃ (350.14 g, 1074.6 mmol), toluene (1 L). The resulting solution was stirred for overnight at 100° C. in an oil bath. The reaction mixture was cooled to 25° C. after reaction completed. The resulting mixture was concentrated under reduced pressure. The residue was applied onto a silica gel column with ethyl acetate/hexane (1:1). Removal of solvent under reduced pressure gave benzyl (S)-4-(5-bromo-6-(1-methoxyethyl)pyridin-3-yl)piperazine-1-carboxylate (135 g, 65.1% yield) as a dark yellow solid. LCMS (ESI): m/z: [M+H] calc'd for C₂₀H₂₄BrN₃O₃ 433.1; found 434.1.

Step 2. Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), Toluene (1 L). The resulting solution was stirred for 2 days at 90° C. in an oil bath. The reaction mixture was cooled to 25° C. The resulting mixture was concentrated under vacuum. The residue was applied onto a neutral alumina column with ethyl acetate/hexane (1:3). Removal of solvent under reduced pressure gave benzyl (S)-4-(6-(1-methoxyethyl)-5-(4,4,5,5-tetramethyl-1,3,2-dioxaborolan-2-yl)pyridin-3-yl)piperazine-1-carboxylate (167 g, crude) as a dark yellow solid. LCMS (ESI): m/z: [M+H] calc'd for C₂₅H₃₆BN₃O₅ 481.3; found 482.1.

Step 3. Into a 3-L 3-necked round-bottom flask purged and maintained with an inert atmosphere of argon, was placed (S)-4-(6-(1-methoxyethyl)-5-(4,4,5,5-tetramethyl-1,3,2-dioxaborolan-2-yl)pyridin-3-yl)piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), Toluene (200 mL). The resulting solution was stirred for overnight at 70° C. in an oil bath. The reaction mixture was cooled to 25° C. after reaction completed. The resulting mixture was concentrated under vacuum. The residue was applied onto a silica gel column with ethyl acetate/hexane (1:1). Removal of solvent under reduced pressure gave benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (146 g, 48.1% yield) as a yellow solid. LCMS (ESI): m/z: [M+H] calc'd for C₄₉H₅₇BrN₄O₄Si 872.3; found 873.3.

Step 4. To a stirred mixture of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (146 g, 167.0 mmol) and Cs₂CO₃ (163.28 g, 501.1 mmol) in DMF (1200 mL) was added C₂H₅₁ (52.11 g, 334.0 mmol) in portions at 0° C. under N₂ atmosphere. The final reaction mixture was stirred at 25° C. for 12 h. Desired product could be detected by LCMS. The resulting mixture was diluted with EA (1 L) and washed with brine (3×1.5 L). The organic layers were dried over anhydrous Na₂SO₄. After filtration, the filtrate was concentrated under reduced pressure to give benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate (143 g, crude) as a yellow solid that was used directly for next step without further purification. LCMS (ESI): m/z [M+H] calc'd for C₅₁H₆₁BrN₄O₄Si 900.4; found 901.4.

Step 5. To a stirred mixture of benzyl benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate (143 g, 158.5 mmol) in DMF (1250 mL) was added CsF (72.24 g, 475.5 mmol). Then the reaction mixture was stirred at 60° C. for 2 days under N₂ atmosphere. Desired product could be detected by LCMS. The resulting mixture was diluted with EA (1 L) and washed with brine (3×1 L). Then the organic phase was concentrated under reduced pressure. The residue was purified by silica gel column chromatography, eluted with PE/EA (1/3) to afford two atropisomers of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate A (38 g, 36% yield, RT=1.677 min in 3 min LCMS (0.1% FA)) and B (34 g, 34% yield, RT=1.578 min in 3 min LCMS (0.1% FA)) both as yellow solid. LCMS (ESI): m/z: [M+H] calc'd for C₃₅H₄₃BrN₄O₄ 663.2; found 662.2.

Step 6. Into a 500-mL 3-necked round-bottom flask purged and maintained with an inert atmosphere of nitrogen, was placed benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate A (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), Toluene (150 mL). The resulting solution was stirred for 5 h at 90° C. in an oil bath. The reaction mixture was cooled to 25° C. The resulting mixture was concentrated under vacuum. The residue was purified by silica gel column chromatography, eluted with PE/EA (1/3) to give benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (12 g, 76.0% yield) as a yellow solid. LCMS (ESI): m/z [M+H] calc'd for C₄₁H₅₅BN₄O₆ 710.4; found 711.3.

Step 7. Into a 250-mL round-bottom flask purged and maintained with an inert atmosphere of argon, was placed benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (10.8 g, 15.2 mmol), methyl (3S)-1-[(2S)-3-(4-bromo-1,3-thiazol-2-yl)-2-[(tert-butoxycarbonyl)amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), Toluene (60 mL), dioxane (20 mL), H₂O (20 mL). The resulting solution was stirred for 3 h at 70° C. in an oil bath. The reaction mixture was cooled to 25° C. The resulting solution was extracted with EtOAc (2×50 mL) and concentrated under reduced pressure. The residue was applied onto a silica gel column with ethyl acetate/hexane (10:1). Removal of solvent to give methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (8 g, 50.9% yield) as a yellow solid. LCMS (ESI): m/z: [M+H] calc'd for C₅₂H₆₈N₈O₉S 980.5; found 980.9.

Step 8. To a stirred mixture of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (12 g, 12.23 mmol) in THF (100 mL)/H₂O (100 mL) was added LiOH (2.45 g, 61.1 mmol) under N₂ atmosphere and the resulting mixture was stirred for 2 h at 25° C. Desired product could be detected by LCMS. THF was concentrated under reduced pressure. The pH of aqueous phase was acidified to 5 with HCL (1N) at 0° C. The aqueous layer was extracted with DCM (3×100 ml). The organic phase was concentrated under reduced pressure to give (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (10 g, 84.5% yield) as a light yellow solid. LCMS (ESI): m/z: [M+H] calc'd for C₅₁H₆₆N₈O₉S 966.5; found 967.0.

Step 9. Into a 3-L round-bottom flask purged and maintained with an inert atmosphere of nitrogen, was placed (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (18 g, 18.61 mmol), ACN (1.8 L), DIEA (96.21 g, 744.4 mmol), EDCI (107.03 g, 558.3 mmol), HOBT (25.15 g, 186.1 mmol). The resulting solution was stirred for overnight at 25° C. The resulting mixture was concentrated under reduced pressure after reaction completed. The resulting solution was diluted with DCM (1 L). The resulting mixture was washed with HCl (3×1 L, 1N aqueous). The resulting mixture was washed with water (3×1 L). Then the organic layer was concentrated, the residue was applied onto a silica gel column with ethyl acetate/hexane (1:1). Removal of solvent under reduced pressure gave benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate (10.4 g, 54.8% yield) as a light yellow solid. LCMS (ESI): m/z: [M+H] calc'd for C₅₁H₆₄N₈O₈S 948.5; found 949.3.

Step 10. Into a 250-mL round-bottom flask purged and maintained with an inert atmosphere of nitrogen, was placed benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate (10.40 g, 10.9 mmol), Pd(OH)₂/C (5 g, 46.9 mmol), MeOH (100 mL). The resulting solution was stirred for 3 h at 25° C. under 2 atm H₂ atmosphere. The solids were filtered out and the filter cake was washed with MeOH (3×100 mL). Then combined organic phase was concentrated under reduced pressure to give tert-butyl ((6³S,4S,Z)-1¹-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.5 g, 90.4% yield) as a light yellow solid. LCMS (ESI): m/z: [M+H] calc'd for C₄₃H₅₈N₈O₆S 814.4; found 815.3.

Step 11. Into a 1000-mL round-bottom flask purged and maintained with an inert atmosphere of nitrogen, was placed tert-butyl ((6³S,4S,Z)-1¹-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate (8.5 g, 10.4 mmol), MeOH (100 mL), AcOH (1.88 g, 31.2 mmol) and stirred for 15 mins. Then HCHO (1.88 g, 23.15 mmol, 37% aqueous solution) and NaBH₃CN (788 mg, 12.5 mmol) was added at 25° C. The resulting solution was stirred for 3 h at 25° C. The resulting mixture was quenched with 100 mL water and concentrated under reduced pressure to remove MeOH. The resulting solution was diluted with 300 mL of DCM. The resulting mixture was washed with water (3×100 mL). Removal of solvent gave tert-butyl ((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.2 g, 90.1% yield) as a yellow solid. LCMS (ESI): m/z: [M+H] calc'd for C₄₄H₆₀N₆O₆S 828.4; found 829.3.

### Example A11. Synthesis of methyl (3S)-3-{[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl](methyl)carbamoyl}pyrrolidine-1-carboxylate

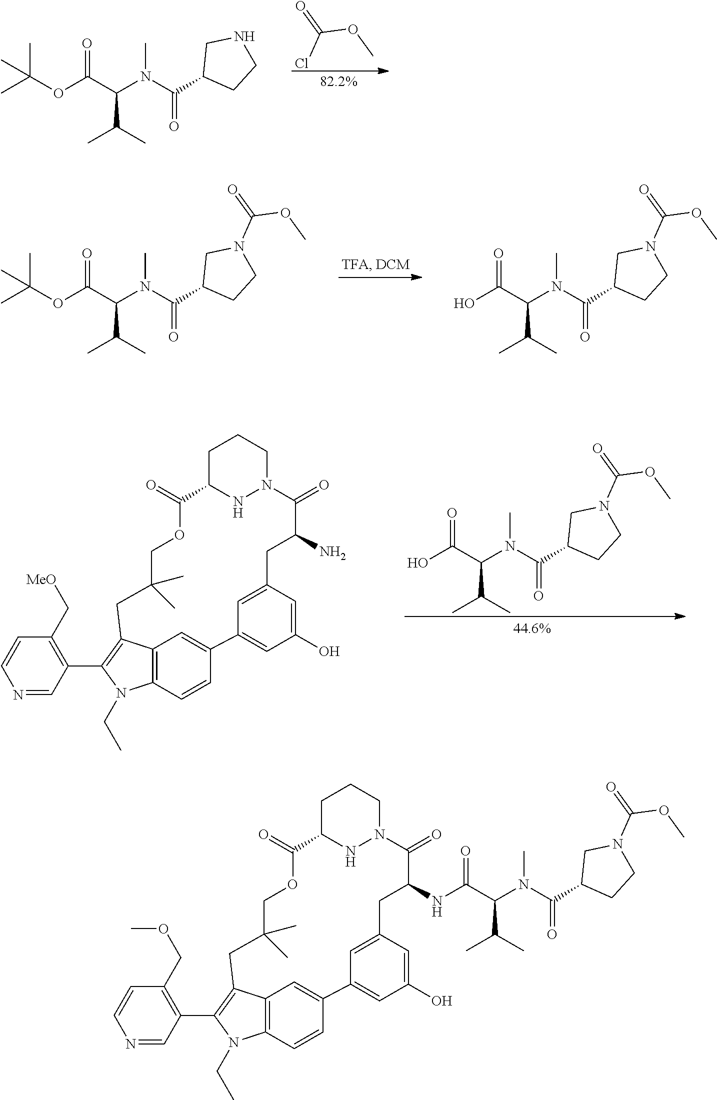

Step 1. To a mixture of tert-butyl N-methyl-N—((S)-pyrrolidine-3-carbonyl)-L-valinate (500 mg, 1.8 mmol) and TEA (356 mg, 3.5 mmol) in DCM (10 mL) at 0° C. was added methyl carbonochloridate (199 mg, 2.1 mmol) dropwise. The mixture was allowed to warm to rt and was stirred for 12 then concentrated under reduced pressure and the residue was purified by silica gel column chromatography to give methyl (S)-3-(((S)-1-(tert-butoxy)-3-methyl-1-oxobutan-2-yl)(methyl)carbamoyl)pyrrolidine-1-carboxylate (550 mg, 82%) as an oil. LCMS (ESI): m/z: [M+H] calc'd for C₁₇H₃₀N₂O₅ 342.2; found 343.2.

Step 2. A mixture of methyl (S)-3-(((S)-1-(tert-butoxy)-3-methyl-1-oxobutan-2-yl)(methyl)carbamoyl)pyrrolidine-1-carboxylate (500 mg, 1.46 mmol), DCM (8 mL) and TFA (2 mL) was stirred at rt for 3 h. The mixture was concentrated under reduced pressure with azeotropic removal of H₂O using toluene (5 mL) to give N—((S)-1-(methoxycarbonyl)pyrrolidine-3-carbonyl)-N-methyl-L-valine (400 mg) as an oil. LCMS (ESI): m/z: [M+H] calc'd for C₁₃H₂₂N₂O₅ 286.2; found 287.2.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (80 mg, 0.13 mmol), N—((S)-1-(methoxycarbonyl)pyrrolidine-3-carbonyl)-N-methyl-L-valine (55 mg, 0.19 mmol) and DIPEA (165 mg, 1.3 mmol) in DMF (2 mL) at 0° C. was added COMU (77 mg, 0.18 mmol). The mixture was stirred at 0° C. for 2 h, then concentrated under reduced pressure and the residue was purified by prep-HPLC to give methyl (3S)-3-{[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl](methyl)carbamoyl}pyrrolidine-1-carboxylate (51 mg, 45% yield) as a solid. LCMS (ESI): m/z: [M+H] calc'd for C₄₉H₅₃N₇O₉ 893.5; found 894.7; ¹H NMR (400 MHz, DMSO-d₆) δ 9.33 (s, 1H), 8.88-8.66 (m, 2H), 8.62 (s, 1H), 8.17-8.06 (m, 1H), 7.92 (d, J=8.7 Hz, 1H), 7.79-7.68 (m, 1H), 7.65-7.49 (m, 2H), 7.21-7.11 (m, 1H), 7.01 (d, J=11.8 Hz, 1H), 6.71-6.40 (m, 1H), 5.54-5.30 (m, 1H), 5.28-4.99 (m, 1H), 4.87-4.56 (m, 1H), 4.46-4.21 (m, 3H), 4.11-3.89 (m, 3H), 3.70 (s, 1H), 3.65-3.59 (m, 4H), 3.35 (s, 2H), 3.24 (s, 2H), 3.18-3.07 (s, 1H), 3.00-2.58 (m, 8H), 2.22-2.01 (m, 4H), 1.81 (d, J=11.4 Hz, 2H), 1.72-1.42 (m, 2H), 1.15-0.64 (m, 13H), 0.43 (d, J=16.4 Hz, 3H).

### Example A17. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-formylpyrrolidin-3-yl]-N-methylformamido}-3-methylbutanamide

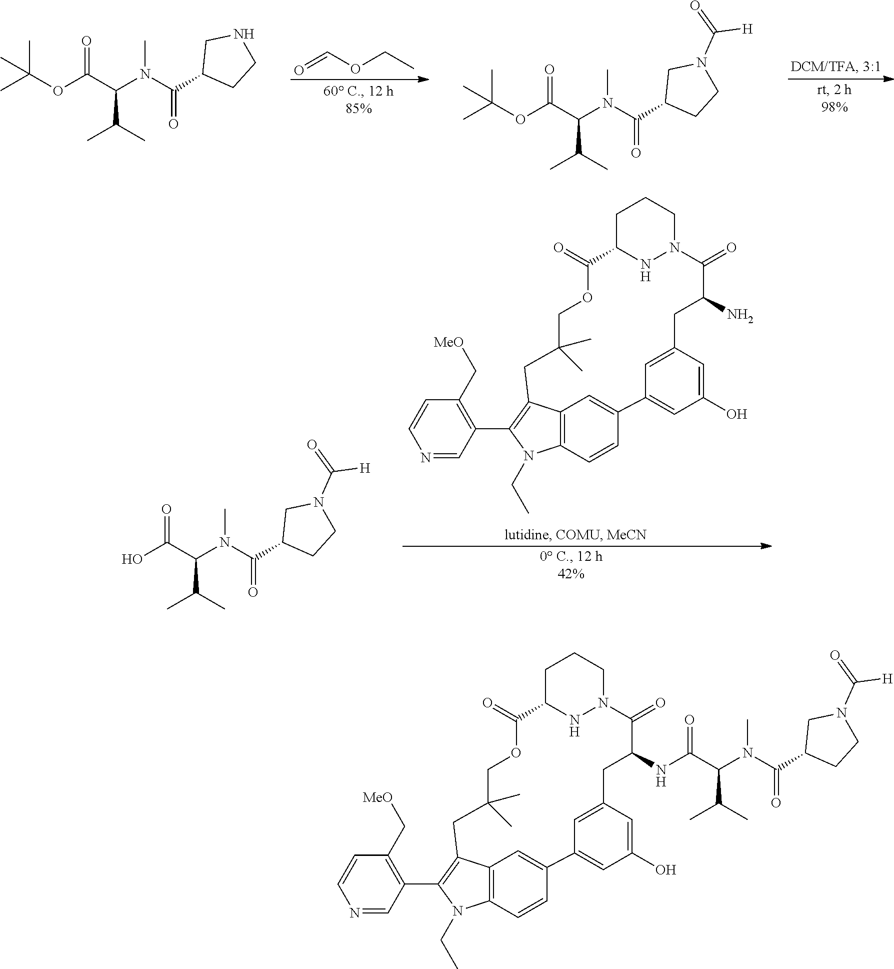

Step 1. A mixture of tert-butyl (2S)-3-methyl-2-[N-methyl-1-(3S)-pyrrolidin-3-ylformamido]butanoate (290 mg, 1.0 mmol) and ethyl formate (755 mg, 10.2 mmol) was heated to 60° C. and stirred for 12 h. The mixture was concentrated under reduced pressure to give tert-butyl (2S)-2-[1-[(3S)-1-formylpyrrolidin-3-yl]-N-methylformamido]-3-methylbutanoate (300 mg, 85% yield) as a solid. LCMS (ESI): m/z: [M+H−tBu] calc'd for C₁₂H₂₀N₂O₄ 256.1; found 257.2.

Step 2. To a mixture of tert-butyl (2S)-2-[1-[(3S)-1-formylpyrrolidin-3-yl]-N-methylformamido]-3-methylbutanoate (290 mg, 0.93 mmol) in DCM (3 mL) at rt was added TFA (1 mL). The mixture was stirred at rt for 2 h, then concentrated under reduced pressure to give (2S)-2-[1-[(3S)-1-formylpyrrolidin-3-yl]-N-methylformamido]-3-methylbutanoic acid (260 mg, 98%) as an oil. LCMS (ESI): m/z: [M+H] calc'd for C₁₂H₂₀N₂O₄ 256.1; found 257.2.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (60 mg, 0.1 mmol), 2,6-dimethylpyridine (15.4 mg, 0.14 mmol) and (2S)-2-[1-[(3S)-1-formylpyrrolidin-3-yl]-N-methylformamido]-3-methylbutanoic acid (37 mg, 0.14 mmol) in MeCN (2 mL) at 0° C. under an atmosphere of N₂ was added COMU (62 mg, 0.14 mmol). The mixture was stirred at 0° C. for 12 h, then concentrated under reduced pressure and the residue was purified by prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-formylpyrrolidin-3-yl]-N-methylformamido}-3-methylbutanamide (35 mg, 42%) as a solid. LCMS (ESI): m/z: [M+H] calc'd for C₄₈H₆₁N₇O₈ 863.5; found 864.5; ¹H NMR (400 MHz, DMSO-6) δ 8.79-8.61 (m, 2H), 8.51 (d, J=7.8 Hz, 3H), 8.31-8.09 (m, 1H), 7.93 (s, 1H), 7.68-7.48 (m, 3H), 7.25-6.97 (m, 2H), 6.71-6.43 (m, 1H), 5.40 (d, J=24.8 Hz, 1H), 5.22 (s, 1H), 4.86-4.34 (m, 1H), 4.23 (t, J=13.8 Hz, 3H), 4.12-3.84 (m, 3H), 3.83-3.54 (m, 4H), 3.22 (d, J=1.7 Hz, 2H), 3.09 (d, J=14.3 Hz, 1H), 3.01-2.92 (m, 1H), 2.99-2.93 (m, 2H), 2.92-2.65 (m, 5H), 2.07 (d, J=12.2 Hz, 4H), 1.80 (s, 1H), 1.74-1.48 (m, 2H), 1.08 (t, J=7.1 Hz, 2H), 1.03-0.54 (m, 12H), 0.43 (d, J=16.2 Hz, 3H).

### Example A6. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-(2-[(3R)-3-hydroxypyrrolidin-1-yl]acetyl)pyrrolidin-3-yl]-N-methylformamido}-3-methylbutanamide

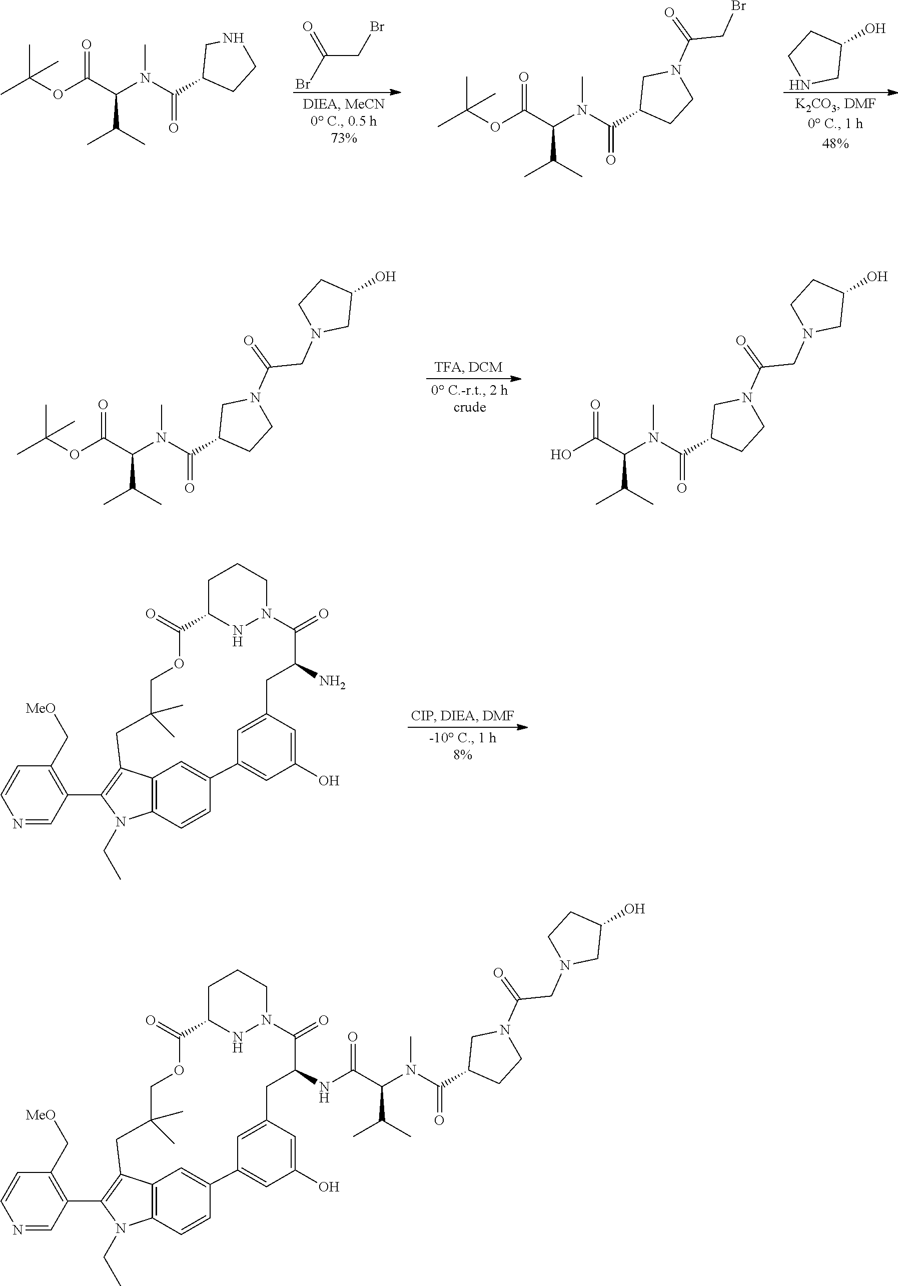

Step 1. A mixture of tert-butyl (2S)-3-methyl-2-[N-methyl-1-(3S)-pyrrolidin-3-ylformamido]butanoate (300 mg, 1.1 mmol) and DIPEA (409 mg, 3.2 mmol) in MeCN (4 mL) at 0° C. was added bromoacetyl bromide (256 mg, 1.3 mmol) dropwise. The mixture was stirred at 0° C. for 30 min, then concentrated under reduced pressure and the residue was purified by C18-silica gel column chromatography to give tert-butyl (2S)-2-[1-[(3S)-1-(2-bromoacetyl)pyrrolidin-3-yl]-N-methylformamido]-3-methylbutanoate (350 mg, 73% yield) as an oil. LCMS (ESI): m/z: [M+H] calc'd for C₁₇H₂₉BrN₂O₄ 404.1; found 405.2 and 407.2.

Step 2. To a mixture of tert-butyl (2S)-2-[1-[(3S)-1-(2-bromoacetyl)pyrrolidin-3-yl]-N-methylformamido]-3-methylbutanoate (110 mg, 0.27 mmol) and K₂CO₃ (75 mg, 0.54 mmol) in DMF (2 mL) at 0° C. was added (3S)-pyrrolidin-3-ol (36 mg, 0.41 mmol) dropwise. The mixture was stirred at 0° C. for 1 h, then concentrated under reduced pressure and the residue was purified by prep-HPLC to give tert-butyl (2S)-2-[1-[(3S)-1-[2-[(3S)-3-hydroxypyrrolidin-1-yl]acetyl]pyrrolidin-3-yl]-N-methylformamido]-3-methylbutanoate (60 mg, 48% yield) as an oil. LCMS (ESI): m/z: [M+H] calc'd for C₂₁H₃₇N₃O₅ 411.3; found 412.5.

Step 3. To a mixture of tert-butyl (2S)-2-[1-[(3S)-1-[2-[(3S)-3-hydroxypyrrolidin-1-yl]acetyl]pyrrolidin-3-yl]-N-methylformamido]-3-methylbutanoate (60 mg, 0.15 mmol) in DCM (0.50 mL) at 0° C. was added TFA (0.50 mL, 6.7 mmol) dropwise. The mixture was warmed to rt and stirred for 2 h, then concentrated under reduced pressure with toluene (×3) to give (2S)-2-[1-[(3S)-1-[2-[(3S)-3-hydroxypyrrolidin-1-yl]acetyl]pyrrolidin-3-yl]-N-methylformamido]-3-methylbutanoic acid (70 mg, crude) as an oil. LCMS (ESI): m/z: [M+H] calc'd for C₁₇H₂₉N₃O₅ 355.2; found 356.4.

Step 4. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (60 mg, 0.1 mmol) and DIPEA (124 mg, 1.0 mmol) in DMF (1 mL) at −10° C. was added (2S)-2-[1-[(3S)-1-[2-[(3S)-3-hydroxypyrrolidin-1-yl]acetyl]pyrrolidin-3-yl]-N-methylformamido]-3-methylbutanoic acid (51 mg, 0.14 mmol) and CIP (40 mg, 0.14 mmol) in portions. The mixture was stirred at −10° C. for 1 h, then diluted with H₂O (30 mL) and extracted with EtOAc (3×10 mL). The combined organic layers were washed with brine (1×10 mL), dried over anhydrous Na₂SO₄ and filtered. The filtrate was concentrated under reduced pressure and the residue was purified by prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-{2-[(3R)-3-hydroxypyrrolidin-1-yl]acetyl}pyrrolidin-3-yl]-N-methylformamido}-3-methylbutanamide (8.6 mg, 8% yield) as a solid. LCMS (ESI): m/z: [M+H] calc'd for C₅₃H₇₀N₈O₉ 962.5; found 963.5; ¹H NMR (400 MHz, CD₃OD) δ 8.70 (td, J=5.1, 1.6 Hz, 1H), 8.66-8.48 (m, 1H), 8.07-7.90 (m, 1H), 7.76 (dd, J=9.9, 5.2 Hz, 1H), 7.61 (tt, J=9.9, 2.0 Hz, 1H), 7.52 (dt, J=8.7, 3.5 Hz, 1H), 7.11-6.97 (m, 1H), 6.62-6.47 (m, 1H), 5.68-5.48 (m, 1H), 4.79 (dt, J=11.2, 9.1 Hz, 1H), 4.53-4.18 (m, 4H), 4.16-3.86 (m, 3H), 3.85-3.56 (m, 7H), 3.55-3.46 (m, 1H), 3.42 (d, J=4.6 Hz, 4H), 3.26-3.01 (m, 3H), 3.01-2.60 (m, 9H), 2.42-2.01 (m, 6H), 1.92 (s, 1H), 1.75 (s, 2H), 1.62 (q, J=12.7 Hz, 1H), 1.26-0.80 (m, 13H), 0.61-0.40 (m, 3H).

### Example A24. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-methanesulfonylpyrrolidin-3-yl]-N-methylformamido}-3-methylbutanamide

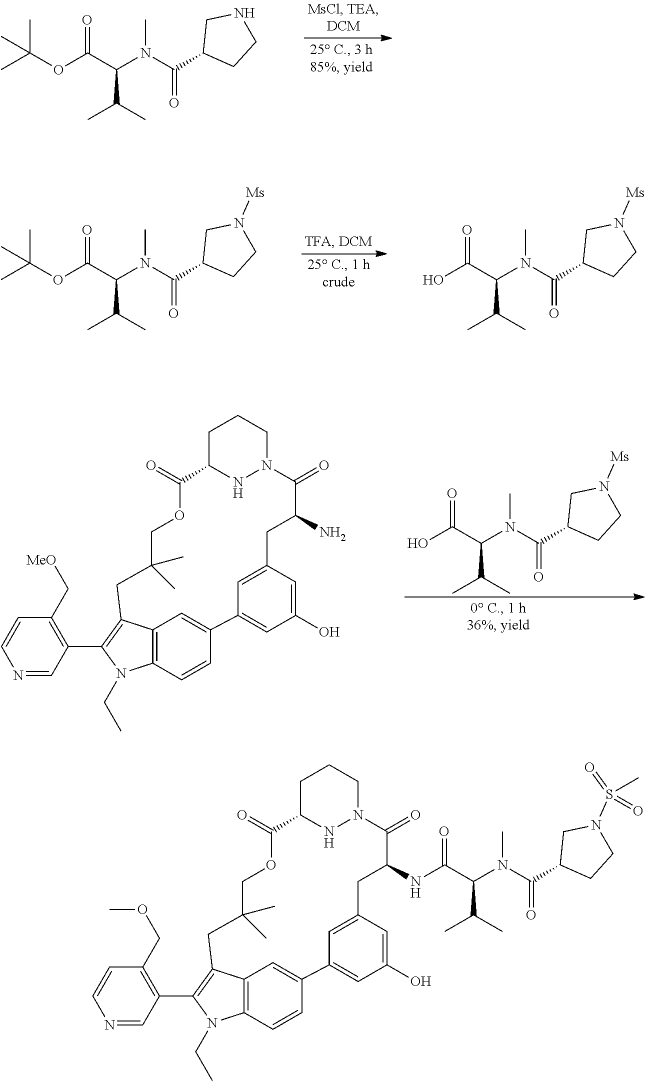 [SMILES: CC(C)[C@@H](C(=O)OC(C)(C)C)N(C)C(=O)[C@H]1CCNC1 ; CC(C)[C@@H](C(=O)OC(C)(C)C)N(C)C(=O)[C@H]1CCN(S(C)(=O)=O)C1 ; CC(C)[C@@H](C(=O)O)N(C)C(=O)[C@H]1CCN(S(C)(=O)=O)C1 ; CCn1c(-c2cnccc2COC)c2c3cc(ccc31)-c1cc(O)cc(c1)C[C@H](N)C(=O)N1CCC[C@H](N1)C(=O)OCC(C)(C)C2 ; CC(C)[C@@H](C(=O)O)N(C)C(=O)[C@H]1CCN(S(C)(=O)=O)C1 ; CCn1c(-c2cnccc2COC)c2c3cc(ccc31)-c1cc(O)cc(c1)C[C@H](NC(=O)[C@H](C(C)C)N(C)C(=O)[C@H]1CCN(S(C)(=O)=O)C1)C(=O)N1CCC[C@H](N1)C(=O)OCC(C)(C)C2]

Step 1. To a mixture of tert-butyl N-methyl-N—((S)-pyrrolidine-3-carbonyl)-L-valinate (500 mg, 1.8 mmol) in DCM (8 mL) at 0° C. under an atmosphere of N₂ was added TEA (356 mg, 3.5 mmol), followed by MsCl (242 mg, 2.1 mmol). The mixture was warmed to rt and was stirred for 3 h, then washed with brine (2×10 mL). The combined organic layers were dried over anhydrous Na₂SO₄, filtered, and the filtrate concentrated under reduced pressure and the residue was by purified by silica gel column chromatography to give tert-butyl N-methyl-N—((S)-1-(methylsulfonyl)pyrrolidine-3-carbonyl)-L-valinate (540 mg, 85%) as an oil. LCMS (ESI): m/z: [M+H] calc'd for C₁₆H₃₀N₂O₅S 362.2; found 363.1.

Step 2. A mixture of tert-butyl N-methyl-N—((S)-1-(methylsulfonyl)pyrrolidine-3-carbonyl)-L-valinate (570 mg, 1.6 mmol), DCM (8 mL) and TFA (2 mL) at rt under an atmosphere of N₂ was stirred for 1 h. The mixture was concentrated under reduced pressure with toluene (5 mL) to give N-methyl-N—((S)-1-(methylsulfonyl)pyrrolidine-3-carbonyl)-L-valine (500 mg) as an oil. LCMS (ESI): m/z: [M+H] calc'd for C₁₂H₂₂N₂O₅S 305.1; found 306.2.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (80 mg, 0.13 mmol) in DMF (2 mL) at 0° C. under an atmosphere of N₂ was added DIPEA (165 mg, 1.3 mmol), N-methyl-N—((S)-1-(methylsulfonyl)pyrrolidine-3-carbonyl)-L-valine (59 mg, 0.19 mmol) and COMU (71 mg, 0.17 mmol). The mixture was stirred at 0° C. for 1 h, then concentrated under reduced pressure and the residue was purified by prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-methanesulfonylpyrrolidin-3-yl]-N-methylformamido}-3-methylbutanamide (42 mg, 36% yield) as a solid. LCMS (ESI): m/z: [M+H] calc'd for C₄₈H₆₃N₇O₉S 913.4; found 914.6; ¹H NMR (400 MHz, DMSO-d₆) δ 9.35-9.33 (m, 1H), 8.74-8.62 (m, 2H), 8.52 (s, 1H), 8.19-8.11 (m, 1H), 7.92 (s, 1H), 7.64-7.60 (m, 2H), 7.53 (t, J=9.0 Hz, 1H), 7.22-7.10 (m, 1H), 7.02 (s, 1H), 6.58-6.48 (m, 1H), 5.37-5.24 (m, 1H), 5.19-5.04 (m, 1H), 4.30-4.18 (m, 3H), 4.07-3.91 (m, 3H), 3.75-3.49 (m, 6H), 3.22 (d, J=1.5 Hz, 2H), 2.97-2.91 (m, 4H), 2.92-2.65 (m, 7H), 2.27 (s, 1H), 2.06 (d, J=14.4 Hz, 3H), 1.85 (d, J=35.3 Hz, 2H), 1.70-1.50 (m, 2H), 1.09-0.88 (m, 8H), 0.85-0.72 (m, 5H), 0.43 (d, J=17.8 Hz, 3H).

### Example A37. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-[(3-hydroxyazetidin-1-yl)sulfonyl]pyrrolidin-3-yl]-N-methylformamido}-3-methylbutanamide

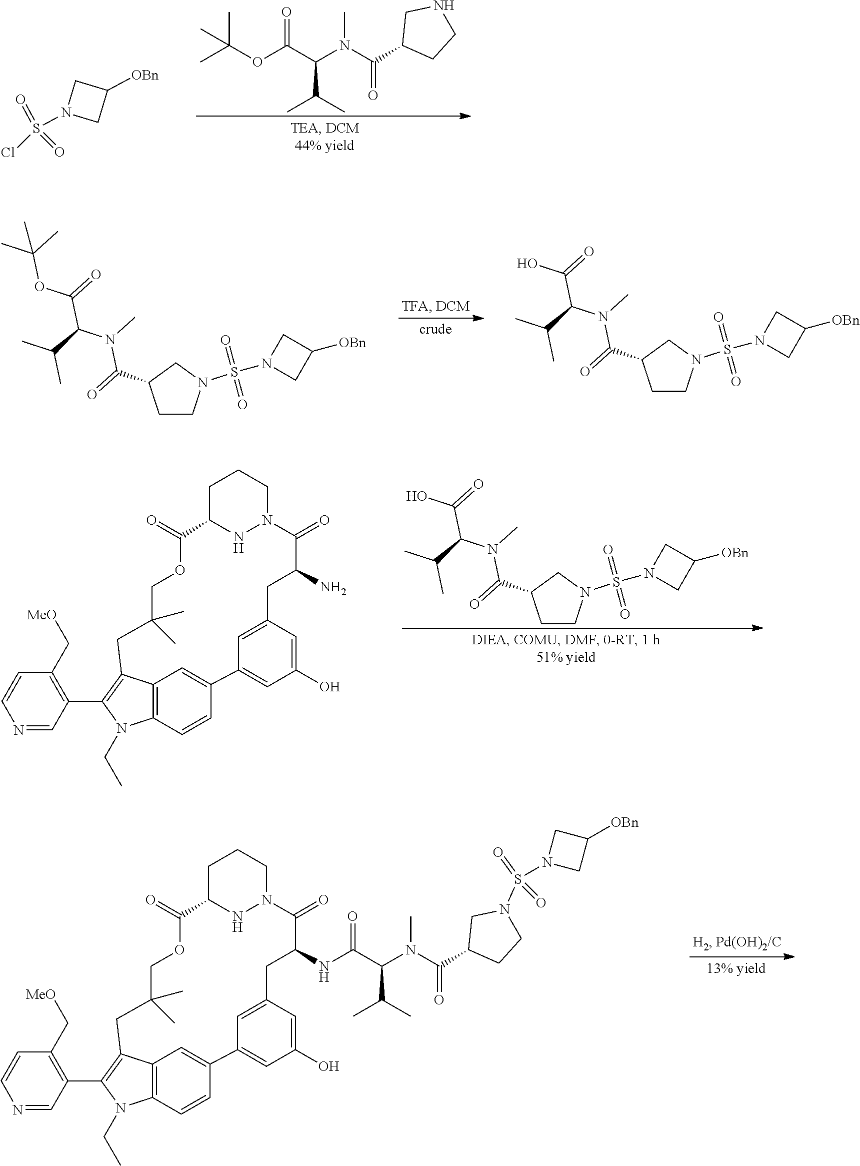

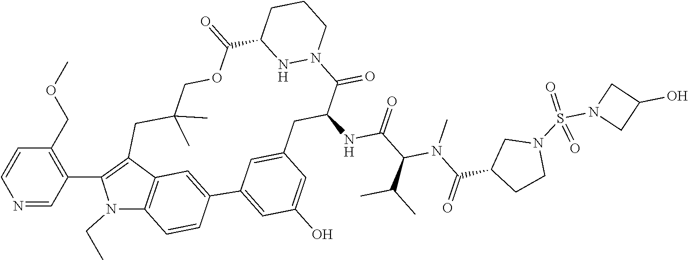 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3CC(O)C3)C1)C(=O)N1CCC[C@H](N1)C(=O)OCC(C)(C)C2]

Step 1. To a mixture of tert-butyl N-methyl-N—((S)-pyrrolidine-3-carbonyl)-L-valinate (500 mg, 1.8 mmol) in DCM (20 mL) ar rt was added TEA (356 mg, 3.5 mmol) and 3-(benzyloxy)azetidine-1-sulfonyl chloride (460 mg, 1.8 mmol). The mixture was stirred at rt overnight, then concentrated under reduced pressure and the residue was purified by prep-HPLC to give tert-butyl N—((S)-1-((3-(benzyloxy)azetidin-1-yl)sulfonyl)pyrrolidine-3-carbonyl)-N-methyl-L-valinate (390 mg, 44% yield) of as an oil. LCMS (ESI): m/z [M+H] calc'd for C₂₅H₃₉N₈O₆S 509.3; found 510.5.

Step 2. A mixture of tert-butyl N—((S)-1-((3-(benzyloxy)azetidin-1-yl)sulfonyl)pyrrolidine-3-carbonyl)-N-methyl-L-valinate (390 mg, 0.77 mmol), DCM (4 mL) and TFA (1 mL) at rt under an atmosphere of N₂ was stirred at rt for 2 h. The mixture was concentrated under reduced pressure with toluene (10 mL×2) to give N—((S)-1-((3-(benzyloxy)azetidin-1-yl)sulfonyl)pyrrolidine-3-carbonyl)-N-methyl-L-valine (370 mg, crude) as a solid. LCMS (ESI): m/z: [M+H] calc'd for C₂₁H₃₁N₃O₆S 453.2; found 454.5.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (60 mg, 0.1 mmol) in DMF (8 mL) at 0° C. under an atmosphere of N₂ was added DIPEA (124 mg, 0.96 mmol), N—((S)-1-((3-(benzyloxy)azetidin-1-yl)sulfonyl)pyrrolidine-3-carbonyl)-N-methyl-L-valine (65 mg, 0.14 mmol) and COMU (58 mg, 0.13 mmol). The mixture was stirred at 0° C. for 1 h, then concentrated under reduced pressure and the residue was purified by prep-HPLC to give (3S)-1-((3-(benzyloxy)azetidin-1-yl)sulfonyl)-N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (52 mg, 51% yield) as an oil. LCMS (ESI): m/z: [M+H] calc'd for C₅₇H₇₂N₈O₁₀S 1060.5; found 1061.3.

Step 4. A mixture of (3S)-1-((3-(benzyloxy)azetidin-1-yl)sulfonyl)-N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (55 mg, 0.05 mmol), MeOH (3 mL) and Pd(OH)₂/C (11 mg, 20% by weight) was stirred under a H₂ atmosphere for 12 h. The mixture was filtered, the filtrate was concentrated under reduced pressure and the residue was purified by prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-[(3-hydroxyazetidin-1-yl)sulfonyl]pyrrolidin-3-yl]-N-methylformamido}-3-methylbutanamide (6.5 mg, 13% yield) as a solid. LCMS (ESI): m/z: [M+H] calc'd for C₅₀H₆₆N₈O₁₀S 970.5; found 971.2; ¹H NMR (400 MHz, DMSO-d₆) δ 9.33-9.29 (m, 1H), 8.75-8.65 (m, 2H), 8.52 (s, 0.5H), 8.15-8.06 (m, 0.5H), 7.92 (s, 1H), 7.65-7.50 (m, 3H), 7.22-7.14 (m, 1H), 7.02 (s, 1H), 6.58-6.46 (m, 1H), 5.84-5.80 (m, 1H), 5.28-5.22 (m, 0.6H), 4.75-4.69 (m, 0.4H), 4.45-4.12 (m, 4H), 4.05-3.88 (m, 5H), 3.72-3.50 (m, 7H), 3.22 (s, 2H), 3.12-3.04 (m, 1H), 2.94-2.70 (m, 7H), 2.29-2.03 (m, 5H), 1.90-1.77 (m, 2H), 1.76-1.45 (m, 2H), 1.24 (s, 1H), 1.08-1.02 (m, 2H), 1.01-0.72 (m, 12H), 0.5-0.43 (m, 3H).

### Example A42. Synthesis of (3S)-N3-[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl]-N1,N1,N3-trimethylpyrrolidine-1,3-dicarboxamide

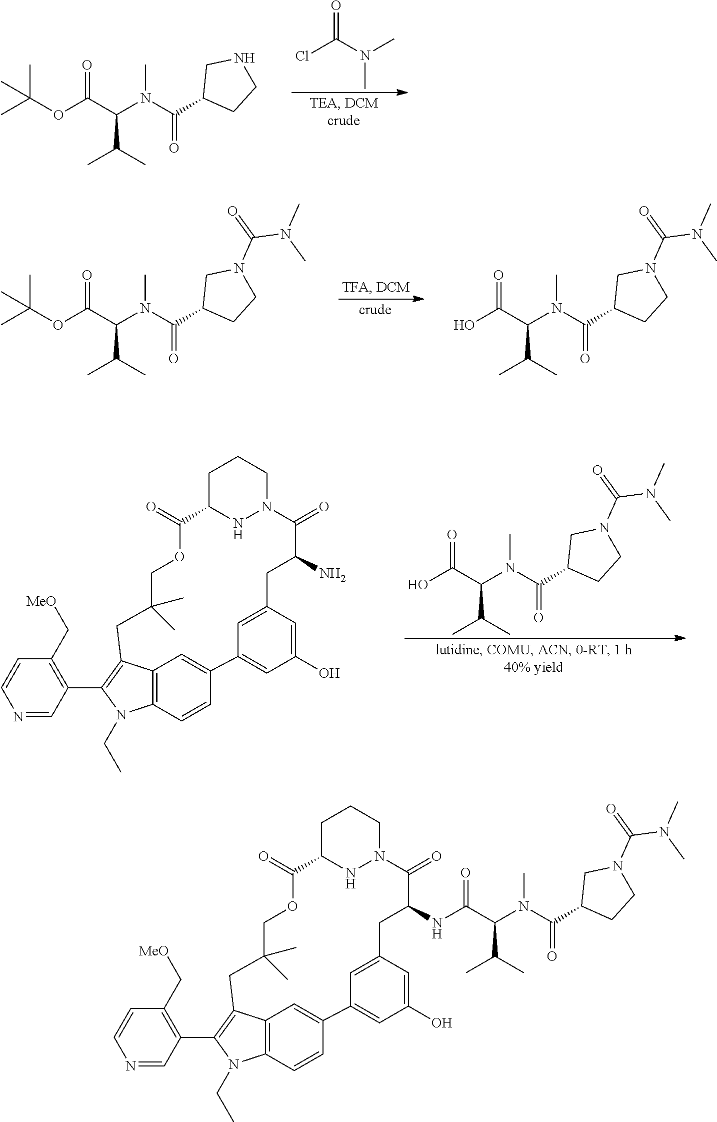

Step 1. To a mixture of tert-butyl (2S)-3-methyl-2-[N-methyl-1-(3S)-pyrrolidin-3-ylformamido]butanoate (200 mg, 0.7 mmol) and TEA (142 mg, 1.4 mmol) in DCM (10 mL) at 0° C. under an atmosphere of N₂ was added dimethylcarbamyl chloride (91 mg, 0.84 mmol) in portions. The mixture was warmed to rt and stirred for 1 h, then H₂O added and the mixture extracted with DCM (3×50 mL). The combined organic layers were washed with brine (1×5 mL), dried over anhydrous Na₂SO₄, filtered and the filtrate concentrated under reduced pressure to give tert-butyl (2S)-2-[1-[(3S)-1-(dimethylcarbamoyl)pyrrolidin-3-yl]-N-methylformamido]-3-methylbutanoate, which was used in the next step without further purification.

Step 2. A mixture of tert-butyl (2S)-2-[1-[(3S)-1-(dimethylcarbamoyl)pyrrolidin-3-yl]-N-methylformamido]-3-methylbutanoate (335 mg, 0.94 mmol) in DCM (10 mL) and TFA (2 mL, 26.9 mmol) was stirred at rt for 2 h. The mixture was concentrated under reduced pressure to give (2S)-2-[1-[(3S)-1-(dimethylcarbamoyl)pyrrolidin-3-yl]-N-methylformamido]-3-methylbutanoic acid, which was used directly in the next step without further purification. LCMS (ESI): m/z: [M+H] calc'd for C₁₄H₂₅N₃O₄ 299.2; found 300.2.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (80 mg, 0.13 mmol) and (2S)-2-[1-[(3S)-1-(dimethylcarbamoyl)pyrrolidin-3-yl]-N-methylformamido]-3-methylbutanoic acid (57 mg, 0.19 mmol) in MeCN (3 mL) at 0° C. under an atmosphere of N₂ was added lutidine (137 mg, 1.3 mmol) and COMU (77 mg, 0.18 mmol) in portions. The mixture was stirred at 0° C. for 1 h, then concentrated under reduced pressure and the residue was purified by prep-HPLC to give (3S)-N3-[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl]-N1,N1,N3-trimethylpyrrolidine-1,3-dicarboxamide (45.6 mg, 39% yield) as a solid. LCMS (ESI): m/z: [M+H] calc'd for C₅₀H₆₆N₈O₈ 906.5; found 907.4; ¹H NMR (400 MHz, DMSO-d₆) δ 9.31-9.30 (m, 1H), 8.72-8.71 (m, 1H), 8.59 (d, J=50.4 Hz, 1H), 7.92-7.90 (m, 1H), 7.74-7.42 (m, 3H), 7.23-7.08 (m, 1H), 7.00 (d, J=13.4 Hz, 1H), 6.56-6.49 (m, 1H), 5.45-5.32 (m, 1H), 5.26-5.04 (m, 1H), 4.87-4.64 (m, 1H), 4.53-4.35 (m, 1H), 4.32-4.09 (m, 3H), 4.12-3.81 (m, 3H), 3.81-3.37 (m, 6H), 3.23 (t, J=1.6 Hz, 2H), 3.12-3.10 (m, 1H), 3.01-2.52 (m, 13H), 2.23-1.95 (m, 4H), 1.81 (s, 1H), 1.67 (s, 1H), 1.60-1.47 (m, 1H), 1.28-1.22 (m, 1H), 1.21-1.14 (m, 1H), 1.11-1.02 (m, 2H), 1.02-0.66 (m, 12H), 0.43 (d, J=16.8 Hz, 3H).

### Example A27. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-methylpyrrolidin-3-yl]formamido}butanamide

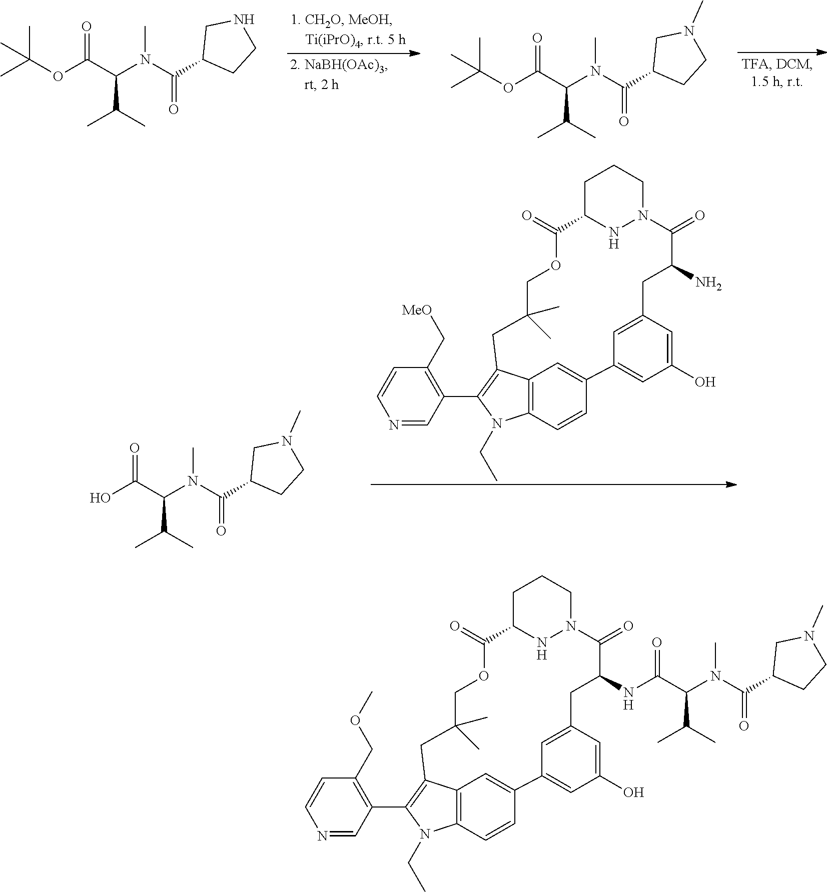

Step 1. A mixture of tert-butyl (2S)-3-methyl-2-[N-methyl-1-(3S)-pyrrolidin-3-ylformamido]butanoate (80 mg 0.28 mmol), Ti(Oi-Pr)₄ (88 mg, 0.31 mmol) and paraformaldehyde (26 mg 0.29 mmol) in MeOH (2 mL) was stirred at rt under an atmosphere of air overnight. The mixture was cooled to 0° C. and NaBH(OAc)₃ (107 mg, 0.51 mmol) was added. The mixture was warmed to rt and stirred for 2 h, then cooled to 0° C. and H₂O (0.2 mL) added. The mixture was concentrated under reduced pressure and the residue was purified by C18-silica gel column chromatography to give tert-butyl (2S)-3-methyl-2-[N-methyl-1-[(3S)-1-methylpyrrolidin-3-yl]formamido]butanoate (97 mg, crude) as an oil. LCMS (ESI): m/z: [M+H] calc'd for C₁₆H₃₀N₂O₃ 298.2; found 299.3.

Step 2. A mixture of tert-butyl (2S)-3-methyl-2-[N-methyl-1-[(3S)-1-methylpyrrolidin-3-yl]formamido]butanoate (97 mg, 0.32 mmol) in DCM (2 mL) and TFA (1 mL, 13.5 mmol) was stirred at rt for 1 h, then the mixture was concentrated under reduced pressure to give (2S)-3-methyl-2-[N-methyl-1-[(3S)-1-methylpyrrolidin-3-yl]formamido]butanoic acid (100 mg, crude) as an oil. LCMS (ESI): m/z: [M+H] calc'd for C₁₂H₂₂N₂O₃ 242.2; found 243.2.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (80 mg, 0.13 mmol) and (2S)-3-methyl-2-[N-methyl-1-[(3S)-1-methylpyrrolidin-3-yl]formamido]butanoic acid (47 mg, 0.19 mmol) in MeCN (2 mL) at 0° C. was added 2,6-dimethylpyridine (137 mg, 1.3 mmol) and COMU (77 mg, 0.18 mmol). The mixture was warmed to rt and stirred for 1 h, then concentrated under reduced pressure and the residue was purified by prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-{N-methyl-1-[(3S)-1-methylpyrrolidin-3-yl]formamido}butanamide (28 mg, 26% yield) as a solid. LCMS (ESI): m/z: [M+H] calc'd for C₄₈H₆₃N₇O₇ 849.5; found 850.5; ¹H NMR (400 MHz, DMSO-d₆) δ 9.31 (s, 1H), 8.72 (t, J=5.1 Hz, 1H), 8.67-8.50 (m, 1H), 7.98-7.87 (m, 1H), 7.67-7.47 (m, 3H), 7.22-7.07 (m, 1H), 7.01 (s, 1H), 6.53 (d, J=40.1 Hz, 1H), 5.44-5.00 (m, 21H), 4.46-4.12 (m, 31H), 4.08-3.79 (m, 3H), 3.79-3.45 (m, 3H), 3.22 (d, J=1.2 Hz, 2H), 3.14-2.94 (m, 2H), 2.92-2.55 (m, 10H), 2.43-2.20 (m, 4H), 2.19-1.92 (m, 4H), 1.81 (d, J=11.9 Hz, 2H), 1.67 (s, 1H), 1.53 (s, 1H), 1.09 (t, J=7.1 Hz, 1H), 1.02-0.91 (m, 3H), 0.91-0.80 (m, 5H), 0.80-0.67 (m, 3H), 0.42 (d, J=21.7 Hz, 3H).

### Example A23. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-(2-hydroxyethyl)pyrrolidin-3-yl]-N-methylformamido}-3-methylbutanamide

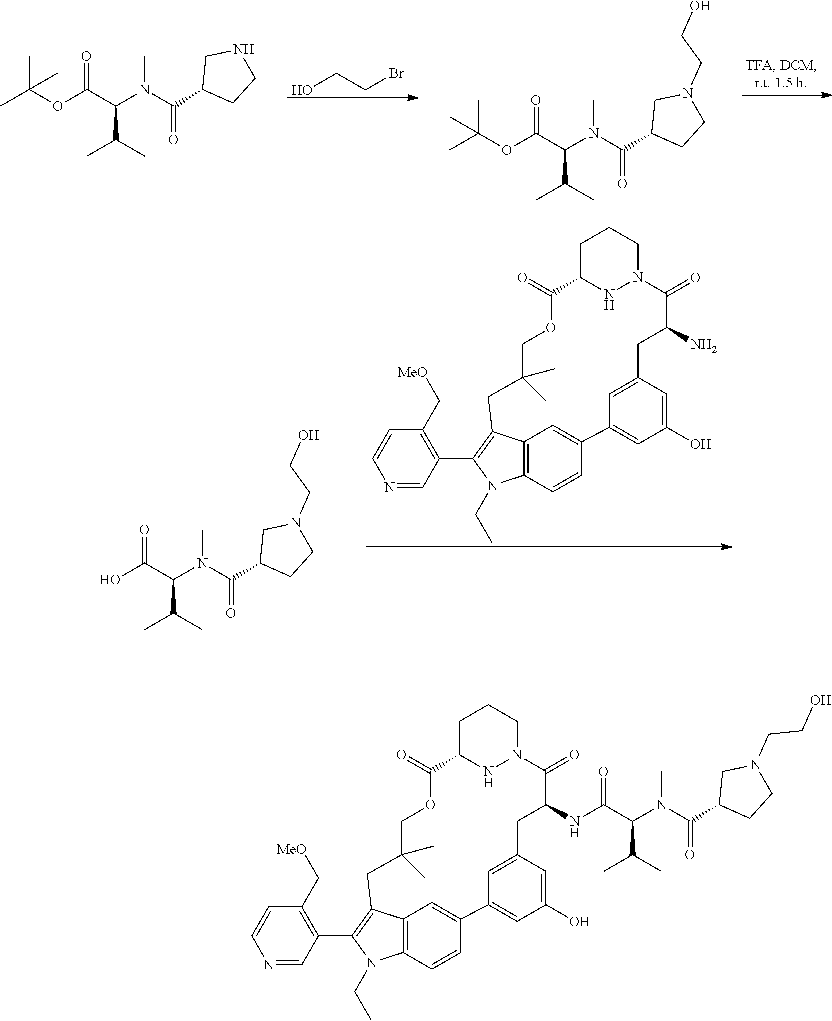

Step 1. To a mixture of tert-butyl (2S)-3-methyl-2-[N-methyl-1-(3S)-pyrrolidin-3-ylformamido]butanoate vanadium (200 mg, 0.6 mmol) and 2-bromoethanol (224 mg, 1.8 mmol) in DMF (5 mL) at rt was added Cs₂CO₃ (777 mg, 2.4 mmol) and KI (50 mg, 0.3 mmol). The mixture was stirred at rt for 16 h then diluted with H₂O and extracted with EtOAc (3×100 mL). The combined organic layers were washed with brine (2×100 mL), dried over anhydrous Na₂SO₄ and filtered. The filtrate was concentrated under reduced pressure and the residue was purified by C18-silica gel column chromatography to give tert-butyl (2S)-2-[1-[(3S)-1-(2-hydroxyethyl)pyrrolidin-3-yl]-N-methylformamido]-3-methylbutanoate (201 mg, crude) as a solid. LCMS (ESI): m/z: [M+H] calc'd for C₁₇H₃₂N₂O₄ 328.2; found 329.4.

Step 2. A mixture of tert-butyl (2S)-2-[1-[(3S)-1-(2-hydroxyethyl)pyrrolidin-3-yl]-N-methylformamido]-3-methylbutanoate (100 mg, 0.3 mmol) in DCM (1 mL) and TFA (0.50 mL) at rt was stirred for 1 h, then concentrated under reduced pressure to give (2S)-2-[1-[(3S)-1-(2-hydroxyethyl)pyrrolidin-3-yl]-N-methylformamido]-3-methylbutanoic acid (110 mg, crude) as an oil. LCMS (ESI): m/z: [M+H] calc'd for C₁₃H₂₄N₂O₄ 272.2; found 273.2.

Step 3. To a mixture of (6³S,4S)-4-amino-1¹-ethyl-2⁵-hydroxy-1²(4(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (60 mg, 0.1 mmol) and (2S)-2-[1-[(3S)-1-(2-hydroxyethyl)pyrrolidin-3-yl]-N-methylformamido]-3-methylbutanoic acid (31 mg, 0.11 mmol) in MeCN (2 mL) at 0° C. under an atmosphere of N₂ was added 2,6-dimethylpyridine (103 mg, 1.0 mmol) and COMU (58 mg, 0.13 mmol). The mixture was warmed to rt and stirred for 1 h, then concentrated under reduced pressure and the residue was purified by prep-HPLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-{1-[(3S)-1-(2-hydroxyethyl)pyrrolidin-3-yl]-N-methylformamido}-3-methylbutanamide (13 mg, 16% yield) as a solid. LCMS (ESI): m/z: [M+H] calc'd for C₄₉H₆₅N₇O₈ 879.5; found 880.3; ¹H NMR (400 MHz, DMSO-d₆) δ 8.72 (t, J=5.3 Hz, 1H), 8.68-8.58 (m, 1H), 8.52 (s, 1H), 7.93 (d, J=10.6 Hz, 1H), 7.68-7.58 (m, 2H), 7.53 (d, J=7.1 Hz, 1H), 7.21-7.07 (m, 1H), 7.01 (s, 1H), 6.52 (d, J=42.8 Hz, 1H), 5.35 (d, J=25.5 Hz, 1H), 5.22-4.97 (m, 1H), 4.59-4.35 (m, 1H), 4.23 (t, J=13.8 Hz, 3H), 4.11-3.81 (m, 3H), 3.81-3.56 (m, 2H), 3.56-3.47 (m, 3H), 3.22 (d, J=1.2 Hz, 2H), 3.09 (d, J=12.6 Hz, 1H), 2.99-2.65 (m, 10H), 2.57-2.53 (m, 1H), 2.47-2.19 (m, 2H), 2.14-2.08 (m, 1H), 2.08 (s, 1H), 2.06-1.98 (m, 2H), 1.81 (s, 2H), 1.59 (d, J=55.9 Hz, 2H), 1.14-0.67 (m, 13H), 0.42 (d, J=22.1 Hz, 3H).

### Example A57. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-(N-methylmethanesulfonamido)butanamide

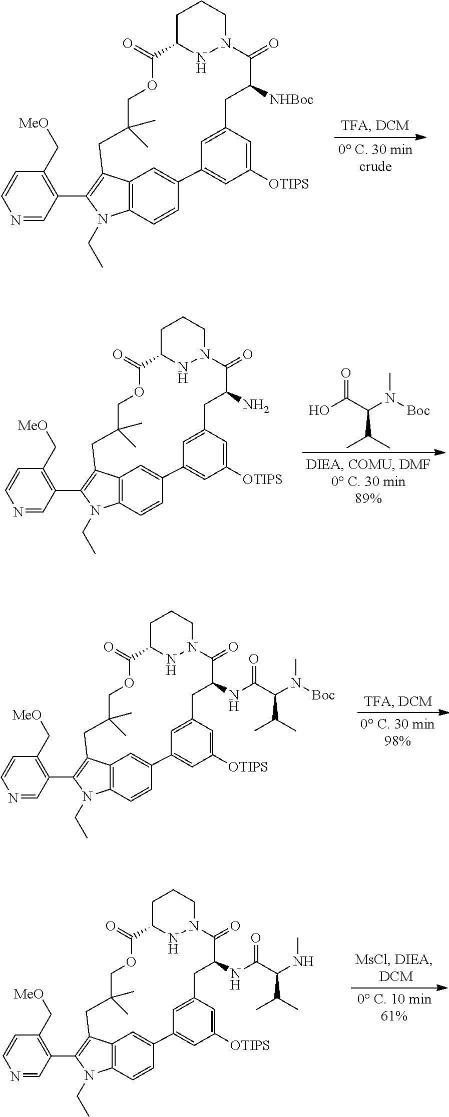

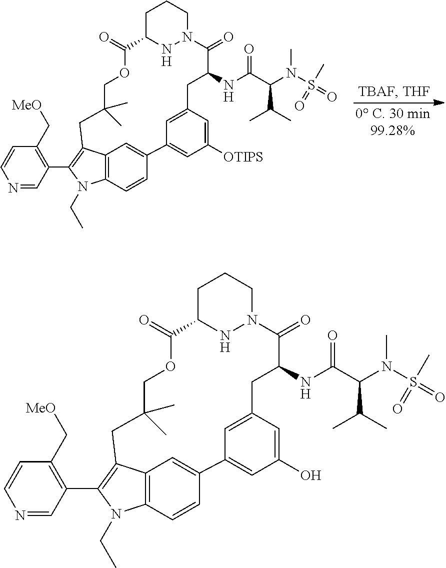

Step 1. A mixture of tert-butyl N-[(8S,14S)-22-ethyl-21-[2-(2-methoxyethyl)phenyl]-18,18-dimethyl-9,15-dioxo-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamate (880 mg, 1.2 mmol), DCM (10 mL) and TFA (5 mL) was stirred at 0° C. for 30 min. The mixture was concentrated under reduced pressure to give (8S,14S)-8-amino-22-ethyl-21-[4-(methoxymethyl)pyridin-3-yl]-18,18-dimethyl-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaene-9,15-dione, that was used directly in the next step without further purification. LCMS (ESI): m/z [M+H] calc'd for C₄₅H₆₃N₅O₅Si 781.5; found 782.7.

Step 2. To a mixture of (8S,14S)-8-amino-22-ethyl-21-[4-(methoxymethyl)pyridin-3-yl]-18,18-dimethyl-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaene-9,15-dione (880 mg, 1.13 mmol) and (2S)-2-[(tert-butoxycarbonyl)(methyl)amino]-3-methylbutanoic acid (521 mg, 2.3 mmol) in DMF (8.8 mL) at 0° C. was added DIPEA (1.45 g, 11.3 mmol) and COMU (88 mg, 0.21 mmol). The mixture was stirred at 0° C. for 30 min, then diluted with H₂O (100 mL) and extracted with EtOAc (3×100 mL). The combined organic layers were washed with brine (3×100 mL), dried over anhydrous Na₂SO₄ and filtered. The filtrate was concentrated under reduced pressure and the residue was purified by prep-TLC to give tert-butyl N-[(1S)-1-[[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamoyl]-2-methylpropyl]-N-methylcarbamate (1 g, 89% yield) as a solid. LCMS (ESI): m/z: [M+H] calc'd for C₅₆H₈₂N₆O₈Si 994.6; found 995.5.

Step 3. A mixture of tert-butyl N-[(1S)-1-[[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamoyl]-2-methylpropyl]-N-methylcarbamate (1.0 g, 1.0 mmol), DCM (10 mL) and TFA (5 mL) was stirred for 30 min. The mixture was concentrated under reduced pressure and the residue was basified to pH ˜8 with saturated NaHCO₃, then extracted with EtOAc (3×10 mL). The combined organic layers were washed with brine (3×10 mL), dried over anhydrous Na₂SO₄, filtered and the filtrate concentrated under reduced pressure to give (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-(methylamino)butanamide (880 mg, 98% yield) as a solid. LCMS (ESI): m/z: [M+H] calc'd for C₅₁H₇₄N₆O₆Si 894.5; found 895.5.

Step 4. To a mixture of (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-(methylamino)butanamide (90 mg, 0.1 mmol) in DCM (2 mL) at 0° C. was added DIPEA (65 mg, 0.5 mmol) and MsCl (14 mg, 0.12 mmol). The mixture was stirred at 0° C. for 30 min, then concentrated under reduced pressure and the residue diluted with H₂O (5 mL) and extracted with EtOAc (3×5 mL). The combined organic layers were washed with brine (3×5 mL), dried over anhydrous Na₂SO₄ and filtered. The filtrate was concentrated under reduced pressure and the residue was purified by prep-TLC to give (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-(N-methylmethanesulfonamido)butanamide (60 mg, 61% yield) as a solid. LCMS (ESI): m/z [M+H] calc'd for C₅₂H₇₆N₆O₈SSi 972.5; found 973.7.

Step 5. To a mixture of (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-(N-methylmethanesulfonamido)butanamide (60 mg, 0.06 mmol) in THF (2 mL) at 0° C. was added 1M TBAF in THF (6 L, 0.006 mmol). The mixture was stirred at 0° C. for 30 min, then diluted with H₂O (5 mL) and extracted with EtOAc (3×5 mL). The combined organic layers were washed with brine (3×5 mL), dried over anhydrous Na₂SO₄ and filtered. The filtrate was concentrated under reduced pressure and the residue was purified by prep-TLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-3-methyl-2-(N-methylmethanesulfonamido)butanamide (50 mg, 99% yield) as a solid. LCMS (ESI): m/z: [M+H] calc'd for C₄₃H₅₆N₆O₈S 816.4; found 817.5; ¹H NMR (400 MHz, DMSO-ab) δ 9.34 (d, J=1.8 Hz, 1H), 8.72 (t, J=5.2 Hz, 1H), 8.65 (d, J=5.8 Hz, 1H), 7.99-7.86 (m, 1H), 7.71-7.45 (m, 3H), 7.19 (d, J=41.5 Hz, 1H), 7.03 (t, J=1.9 Hz, 1H), 6.66 (d, J=10.4 Hz, 1H), 5.34 (q, J=8.1 Hz, 1H), 5.14 (dd, J=62.7, 12.2 Hz, 1H), 4.55-4.15 (m, 3H), 4.14-3.80 (m, 4H), 3.80-3.46 (m, 3H), 3.23 (s, 1H), 3.02-2.72 (m, 8H), 2.68 (s, 2H), 2.15-1.89 (m, 3H), 1.82 (d, J=12.4 Hz, 1H), 1.76-1.62 (m, 1H), 1.54 (q, J=12.7 Hz, 1H), 1.24 (s, 1H), 1.08 (t, J=7.1 Hz, 2H), 1.03-0.86 (m, 9H), 0.81 (s, 2H), 0.46 (s, 3H).

### Example A43. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-(2-hydroxy-N-methylacetamido)-3-methylbutanamide

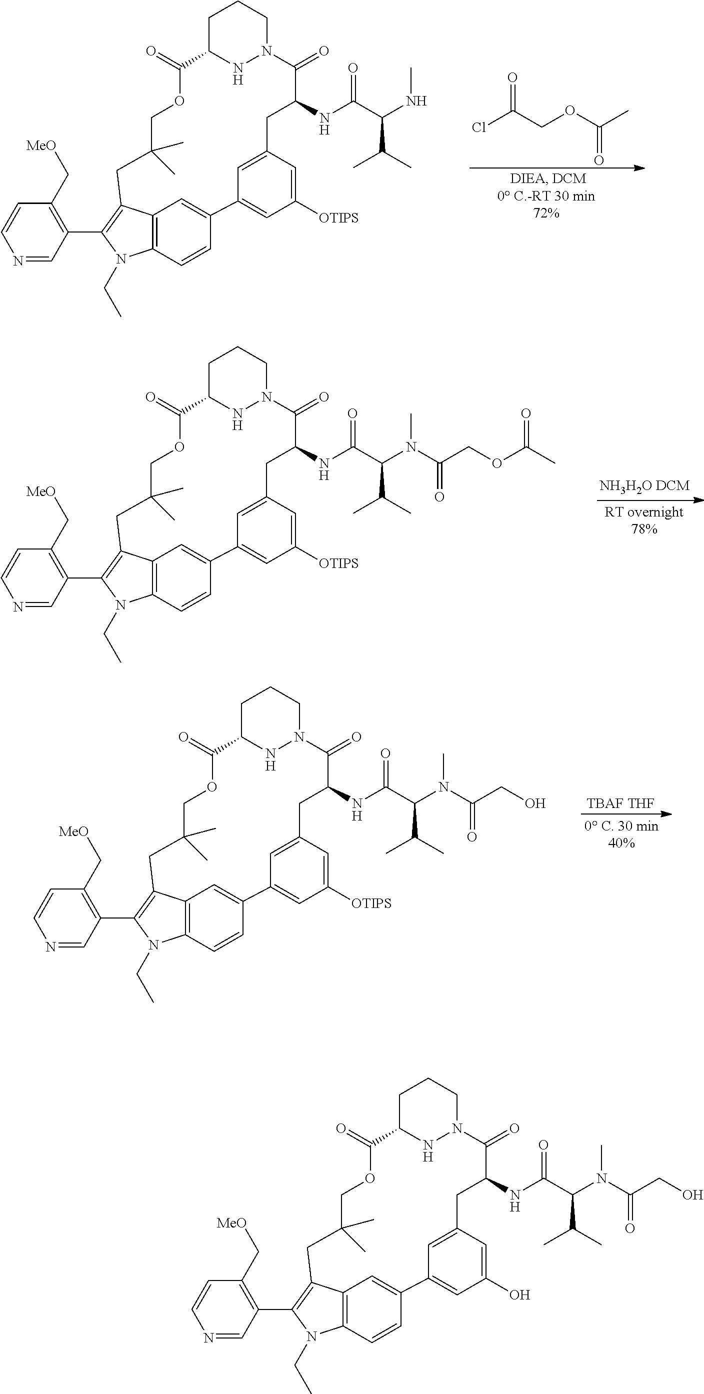

Step 1. To a mixture of (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-3-methyl-2-(methylamino)butanamide (100 mg, 0.11 mmol) in DCM (1 mL) at 0° C. was added DIPEA (72 mg, 0.56 mmol) and 2-chloro-2-oxoethyl acetate (11.53 mg, 0.11 mmol). The mixture was warmed to rt and stirred for 30 min, then concentrated under reduced pressure, diluted with water (3 mL) and extracted with EtOAc (3×3 mL). The combined organic layers were washed with brine (3×3 mL), dried over anhydrous Na₂SO₄ and filtered. The filtrate was concentrated under reduced pressure and the residue was purified by prep-TLC to give [[(1S)-1-[[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamoyl]-2-methylpropyl](methyl)carbamoyl]methyl acetate (80 mg, 72% yield) as a solid. LCMS (ESI): m/z: [M+H] calc'd for C₅₅H₇₈N₆O₉Si 994.6; found 995.7.

Step 2. A mixture of [[(1S)-1-[[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]carbamoyl]-2-methylpropyl](methyl)carbamoyl]methyl acetate (80 mg, 0.080 mmol), DCM (1 mL) and aqueous NH₄OH (0.8 mL) was stirred at rt overnight. H₂O (5 mL) was added and the mixture was extracted with EtOAc (3×5 mL). The combined organic layers were washed with brine (3×5 mL), dried over anhydrous Na₂SO₄ and filtered. The filtrate was concentrated under reduced pressure and the residue was purified by prep-TLC to give (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-2-(2-hydroxy-N-methylacetamido)-3-methylbutanamide (60 mg, 78% yield) as a solid. LCMS (ESI): m/z [M+H] calc'd for C₅₃H₇₆N₆O₈Si 952.6; found 953.7.

Step 3. A mixture of (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6].1\hat{ }[10,14].0\hat{ }[23,27]]nonacosa-1(26),2,4,6(29),20,23(27),24-heptaen-8-yl]-2-(2-hydroxy-N-methylacetamido)-3-methylbutanamide (60 mg, 0.06 mmol), THF (2 mL) and 1M TBAF in THF (6 L, 0.006 mmol) at 0° C. was stirred for 30 min. H₂O (3 mL) was added and the mixture was extracted with EtOAc (3×3 mL). The combined organic layers were washed with brine (3×3 mL), dried over anhydrous Na₂SO₄. The filtrate was concentrated under reduced pressure and the residue was purified by prep-TLC to give (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]-2-(2-hydroxy-N-methylacetamido)-3-methylbutanamide (20 mg, 40% yield) as a solid. LCMS (ESI): m/z: [M+H] calc'd for C₄₄H₅₆N₆O₈ 796.4; found 797.6; ¹H NMR (400 MHz, CD₃OD) δ 8.70 (dd, J=5.7, 4.4 Hz, 1H), 8.66-8.49 (m, 1H), 8.00 (dd, J=4.6, 1.7 Hz, 1H), 7.76 (dd, J=9.9, 5.2 Hz, 1H), 7.60 (dt, J=8.7, 1.6 Hz, 1H), 7.56-7.47 (m, 1H), 7.29-7.18 (m, 1H), 7.10-6.98 (m, 1H), 6.54 (dt, J=3.6, 1.7 Hz, 1H), 5.67-5.55 (m, 1H), 4.77 (dd, J=11.2, 8.4 Hz, 1H), 4.57-4.39 (m, 3H), 4.39-4.20 (m, 3H), 4.19-3.91 (m, 2H), 3.90-3.65 (m, 3H), 3.60 (dd, J=11.0, 1.8 Hz, 1H), 3.42 (s, 1H), 3.32 (s, 1H), 3.29-3.15 (m, 1H), 3.10-2.97 (m, 1H), 2.97-2.82 (m, 5H), 2.82-2.63 (m, 2H), 2.35-2.11 (m, 3H), 1.94 (d, J=13.2 Hz, 1H), 1.82-1.49 (m, 3H), 1.31 (s, 1H), 1.19 (t, J=7.2 Hz, 2H), 1.09-0.95 (m, 7H), 0.95-0.83 (m, 5H), 0.50 (d, J=32.4 Hz, 3H).

### Example A50. Synthesis of oxolan-3-yl-N-[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl]-N-methylcarbamate

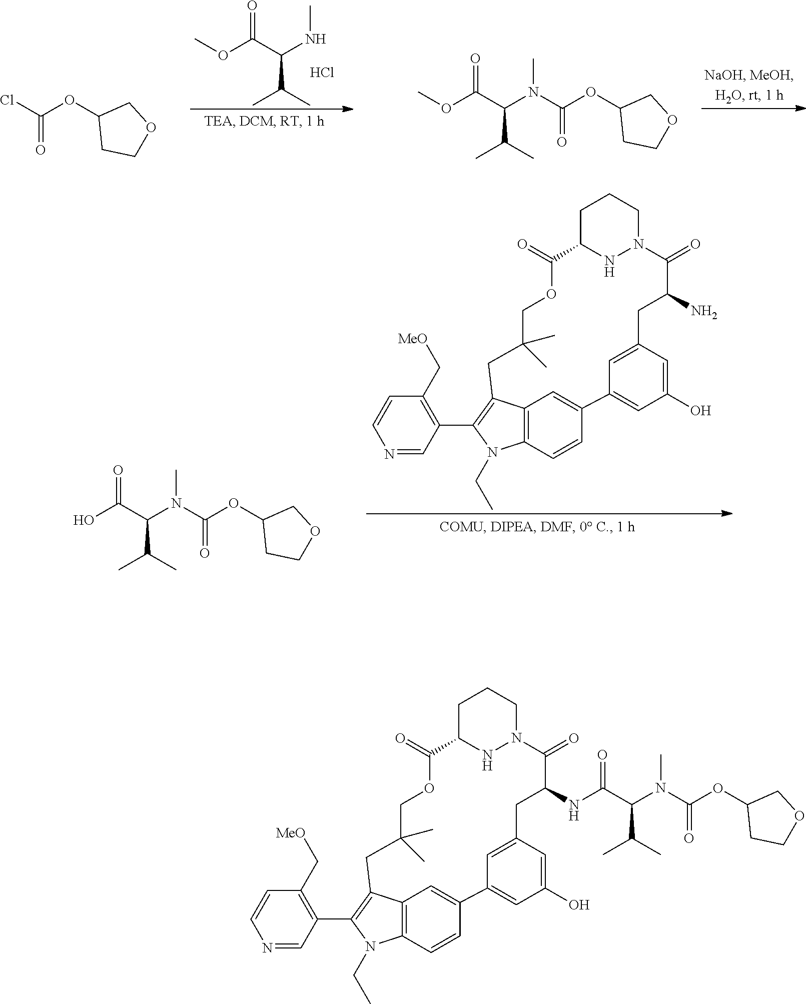

Step 1. To a mixture of methyl (2S)-3-methyl-2-(methylamino)butanoate (500 mg, 3.4 mmol) and TEA (1.44 mL, 14.2 mmol) in DCM (20 mL) at rt was added oxolan-3-yl carbonochloridate (1.04 g, 6.9 mmol). The mixture was stirred at rt for 1 h, then sat. NH₄Cl added and the mixture extracted with DCM (3×10 mL). The combined organic layers were washed with brine (1×10 mL), dried over anhydrous Na₂SO₄ and filtered. The filtrate was concentrated under reduced pressure and the residue was purified by silica gel column chromatography to give methyl (2S)-3-methyl-2 [methyl (oxolan-3-yloxy)carbonyl]amino]butanoate (800 mg, 89% yield) as an oil. ¹H NMR (300 MHz, CDCl₃) δ 4.57-4.05 (m, 1H), 3.99-3.78 (m, 4H), 3.70 (s, 3H), 3.26 (s, 1H), 2.99-2.68 (m, 3H), 2.26-1.83 (m, 3H), 1.06-0.76 (m, 6H).

Step 2. A mixture of methyl (2S)-3-methyl-2 [methyl (oxolan-3-yloxy)carbonyl]amino]butanoate (1 g, 3.9 mmol) and 2M NaOH (19.3 mL, 38.6 mmol) in MeOH (20 mL) was stirred at rt for 1 h. The mixture was concentrated under reduced pressure and the residue was extracted with MTBE (3×10 mL). The aqueous layer was acidified to pH ˜2 with 2 M HCl then extracted with DCM (3×20 mL). The combined organic layers were washed with brine (2×10 mL), dried over anhydrous Na₂SO₄, filtered and the filtrate was concentrated under reduced pressure to give (2S)-3-methyl-2-[methyl[(oxolan-3-yloxy)carbonyl]amino]butanoic acid (630 mg, 67% yield) as an oil. ¹H NMR (300 MHz, CDCl₃) δ 5.32 (br. s, 1H), 4.45-4.08 (m, 1H), 4.04-3.81 (m, 4H), 2.93 (d, J=6.9 Hz, 3H), 2.38-1.93 (m, 3H), 1.06 (t, J=5.6 Hz, 3H), 0.94 (d, J=6.7 Hz, 3H).

Step 3. To a mixture of (6³S,4S)-4-amino-1¹-ethyl-2⁵-hydroxyl-1²-(4-(methoxymethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (80 mg, 0.13 mmol), (2S)-3-methyl-2-[methyl[(oxolan-3-yloxy)carbonyl]amino]butanoic acid (63 mg, 0.26 mmol) and DIPEA (165 mg, 1.3 mmol) in DMF (2 mL) at 0° C. was added COMU (38 mg, 0.19 mmol). The mixture was stirred at 0° C. for 30 min, then the mixture was concentrated under reduced pressure and the residue was purified by prep-HPLC to give oxolan-3-yl-N-[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl}-2-methylpropyl]-N-methylcarbamate (50 mg, 45% yield) as a solid. LCMS (ESI): m/z [M+H] calc'd for C₄₇H₆₀N₆O₉ 852.4; found 853.5; ¹H NMR (400 MHz, DMSO-d₆) δ 9.34-9.18 (m, 1H), 8.72 (t, J=5.1 Hz, 1H), 8.58 (d, J=47.8 Hz, 1H), 8.48-8.15 (m, 1H), 7.91 (s, 1H), 7.70-7.57 (m, 2H), 7.55-7.46 (m, 1H), 7.13 (d, J=24.7 Hz, 1H), 7.01 (s, 1H), 6.56 (d, J=9.2 Hz, 1H), 5.34 (s, 1H), 5.28-5.00 (m, 2H), 4.40 (d, J=13.3 Hz, 1H), 4.33-4.14 (m, 4H), 4.12-3.45 (m, 10H), 3.23 (s, 1H), 3.10 (d, J=14.5 Hz, 1H), 2.99-2.62 (m, 6H), 2.20-1.99 (m, 4H), 1.80 (s, 1H), 1.66 (s, 1H), 1.52 (d, J=12.2 Hz, 1H), 1.09 (t, J=7.1 Hz, 2H), 0.99-0.89 (m, 6H), 0.87-0.76 (m, 5H), 0.42 (d, J=24.2 Hz, 3H).

### Example A277. The synthesis of (2S)—N-((6³S,4S,Z)-1¹-ethyl-1²-(2-((S)-1-methoxyethyl)-5-(4-methylpiperazin-1-yl)pyridin-3-yl)-10,10-trimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-(1,3,3-trimethylureido)butanamide

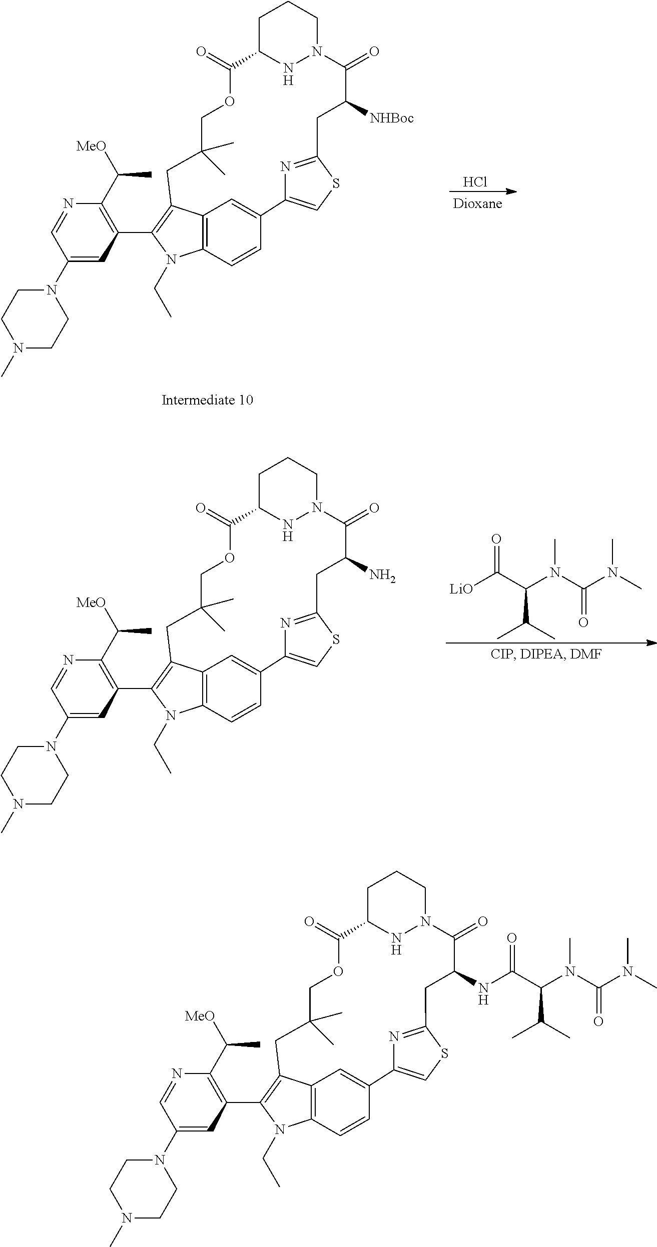

Step 1. A solution of Intermediate 10 (8.2 g, 9.89 mmol) in dioxane (40 mL) at 0° C. under nitrogen atmosphere, was added HCl (40 mL, 4M in dioxane). The reaction solution was stirred at 0° C. for 1 h, then concentrated under reduced pressure. The resulting mixture was diluted with DCM (600 mL) and saturated sodium bicarbonate aqueous solution (400 mL). The organic phase was separated and washed with brine (500 mL×2), then concentrated under reduced pressure to afford (6³S,4S,Z)-4-amino-1¹-ethyl-1²-(2-((S)-1-methoxyethyl)-5-(4-methylpiperazin-1-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (7.2 g, 94.8% yield) as solid. LCMS (ESI): m/z: [M+H] calc'd for C₃₉H₅₂N₈O₄S 728.4; found 729.3.

Step 2. A mixture of (6³S,4S,Z)-4-amino-1¹-ethyl-1²-(2-((S)-1-methoxyethyl)-5-(4-methylpiperazin-1-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (6 g, 8.23 mmol) and lithium N-(dimethylcarbamoyl)-N-methyl-L-valinate (4.28 g, 20.58 mmol) in DMF (80 mL), was added DIEA (53.19 g, 411.55 mmol). The reaction mixture was stirred for 5 minutes, then added CIP (3.43 g, 12.35 mmol) in one portion. The resulting solution was stirred at 25° C. for 1 h, then quenched with water (100 mL), extracted with EtOAc (300 mL). The organic layer was separated and washed with saturated ammonium chloride aqueous solution (100 mL×3) and water (100 mL×2). The combined organic layers were concentrated under reduced pressure. The residue was purified by reverse phase chromatography to afford (2S)—N-((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-(1,3,3-trimethylureido)butanamide (2.5 g, 33.2% yield) as a solid. ¹H NMR (400 MHz, DMSO-d₆) δ 8.52-8.34 (m, 3H), 7.82 (s, 1H), 7.79-7.69 (m, 1H), 7.60-7.50 (m, 1H), 7.26-7.16 (m, 1H), 5.64-5.50 (m, 1H), 5.20-5.09 (m, 1H), 4.40-4.08 (m, 5H), 3.92-3.82 (m, 1H), 3.66-3.50 (m, 2H), 3.37-3.35 (m, 1H), 3.30-3.28 (m, 1H), 3.28-3.20 (m, 4H), 3.19-3.15 (m, 3H), 3.12-3.04 (m, 1H), 2.99-2.89 (m, 1H), 2.81 (s, 6H), 2.77 (s, 4H), 2.48-2.38 (m, 5H), 2.22 (s, 3H), 2.16-2.04 (m, 2H), 1.88-1.78 (m, 2H), 1.60-1.45 (m, 2H), 1.39-1.29 (m, 3H), 0.97-0.80 (m, 12H), 0.34 (s, 3H). LCMS (ESI): m/z: [M+H] calc'd for C₄₈H₆₈N₁₀O₆S 912.5; found 913.6.

### Example A265. The synthesis of N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-4-methylpiperazine-1-carboxamide

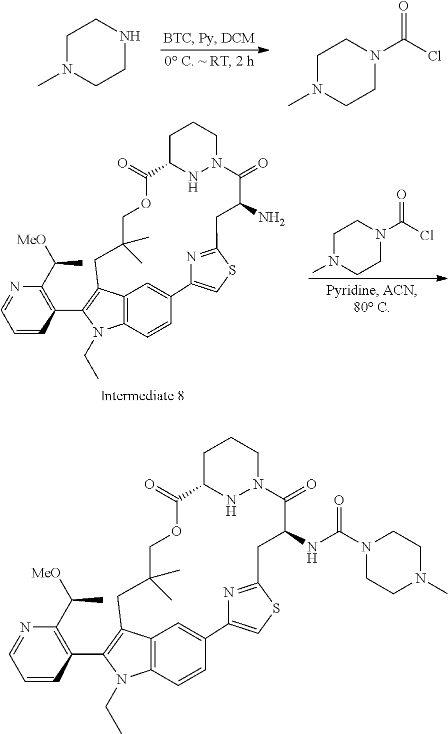

Step 1. To a stirred solution of 1-methylpiperazine (100 mg, 1.148 mmol) and Pyridine (275.78 mg, 3.44 mmol) in DCM (3 mL) were added BTC (112.5 mg, 0.38 mmol) in DCM (1 mL) dropwise at 0° C. under nitrogen atmosphere. The reaction was stirred for 2 hh 0° C. under nitrogen atmosphere. The resulting mixture was concentrated under reduced pressure to afford 4-methylpiperazine-1-carbonyl chloride (250 mg, crude) as an oil.

Step 2. To a stirred solution of Intermediate 8 (100 mg, 0.16 mmol) and pyridine (100 mg, 1.272 mmol) in ACN (2 mL) was added 4-methylpiperazine-1-carbonyl chloride (38.67 mg, 0.24 mmol) dropwise at 0° C. under nitrogen atmosphere. The reaction mixture was stirred for 2 hh at 0° C. under nitrogen atmosphere. The resulting mixture was diluted with water (100 mL) and extracted with EtOAc (100 mL×3). The combined organic layers were washed with brine (50 mL×3), dried over anhydrous Na₂SO₄, then filtered and concentrated under reduced pressure. The residue was purified by reverse flash chromatography to give N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-4-methylpiperazine-1-carboxamide (20 mg, 16.7% yield) as a solid. ¹H NMR (400 MHz, DMSO-d₆) δ 8.76 (dd, J=4.8, 1.7 Hz, 1H), 8.50 (s, 1H), 8.14 (d, J=2.5 Hz, 1H), 7.79 (d, J=9.1 Hz, 2H), 7.77-7.72 (m, 1H), 7.58 (d, J=8.6 Hz, 1H), 7.52 (dd, J=7.7, 4.7 Hz, 1H), 6.82 (d, J=9.0 Hz, 1H), 5.32 (t, J=9.0 Hz, 1H), 4.99 (d, J=12.1 Hz, 1H), 4.43-4.02 (m, 5H), 3.57 (d, J=3.1 Hz, 2H), 3.26 (d, J=8.4 Hz, 6H), 2.97 (d, J=14.3 Hz, 1H), 2.80-2.66 (m, 1H), 2.55 (s, 1H), 2.40 (d, J=14.4 Hz, 1H), 2.32 (d, J=5.9 Hz, 4H), 2.21 (s, 3H), 2.09 (d, J=12.1 Hz, 1H), 1.77 (d, J=18.8 Hz, 2H), 1.52 (dd, J=11.8, 5.4 Hz, 1H), 1.37 (d, J=6.0 Hz, 3H), 1.24 (s, 1H), 0.90 (s, 3H), 0.85 (t, J=7.0 Hz, 3H), 0.32 (s, 3H). LCMS (ESI): m/z: [M+H] calc'd for C₄₀H₅₂N₈O₅S 756.38; found 757.3.

### Example A598. The synthesis of (2S)—N-((6³S,3S,4S,Z)-1¹-ethyl-3-methoxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-(1,3,3-trimethylureido)butanamide

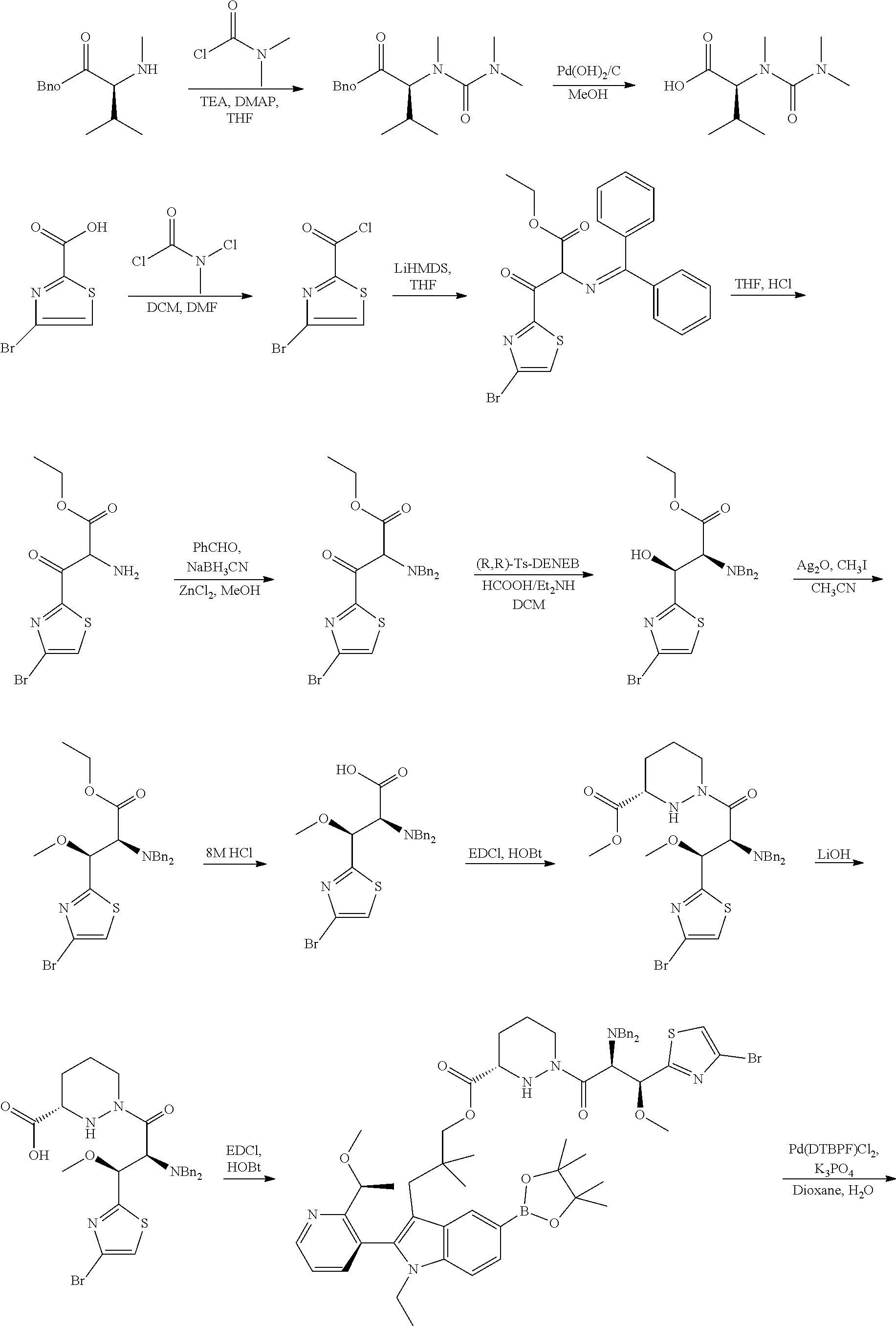

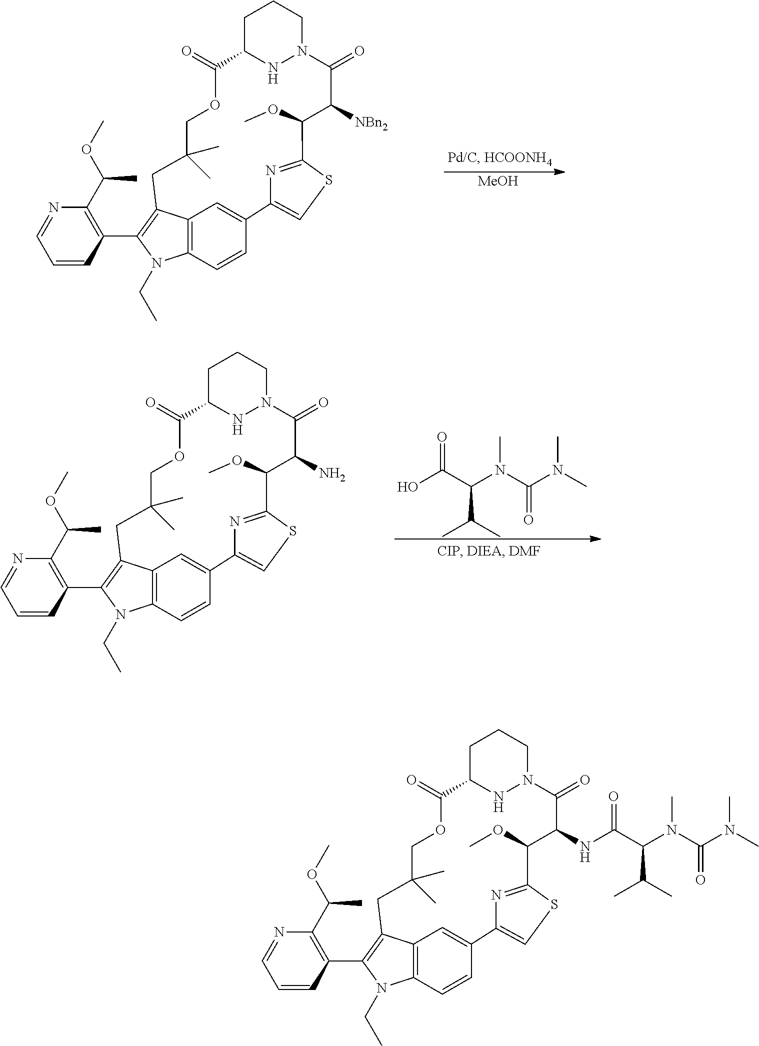

Step 1. A mixture of benzyl (2S)-3-methyl-2-(methylamino)butanoate (500 mg, 2.26 mmol) and dimethylcarbamyl chloride (1.215 g, 11.3 mmol) in THF (5 mL), was added TEA (2.286 g, 22.59 mmol) and DMAP (276.02 mg, 2.26 mmol) in portions under nitrogen atmosphere. The reaction mixture was stirred at 65° C. for 12 hh under nitrogen atmosphere, then quenched with water (100 mL) and was extracted with EtOAc (50 mL×3). The combined organic phase was dried over anhydrous sodium sulfate, filtered and concentrated under reduced pressure. The residue was purified by reverse phase chromatography to afford benzyl N-(dimethylcarbamoyl)-N-methyl-L-valinate (400 mg, 58.3% yield) as an oil. LCMS (ESI): m/z: [M+H] calc'd for C₁₆H₂₄N₂O₃ 292.2; found 293.1.

Step 2. A mixture of benzyl N-(dimethylcarbamoyl)-N-methyl-L-valinate (400 mg, 1.37 mmol) and palladium hydroxide on carbon (400 mg, 2.85 mmol) in MeOH (10 mL) was stirred for 4 hh under hydrogen atmosphere. The reaction mixture was filtered and the filter cake was washed with MeOH (100 mL×3). The filtrate was concentrated under reduced pressure to afford N-(dimethylcarbamoyl)-N-methyl-L-valine (200 mg, crude) as an oil. LCMS (ESI): m/z: [M+H] calc'd for C₉H₁₈N₂O₃ 202.1; found 203.1.

Step 3. A solution of 4-bromo-1,3-thiazole-2-carboxylic acid (10 g, 48.07 mmol) in DCM (100 mL), was added oxalyl chloride (16.27 mL, 192.28 mmol) and DMF (0.11 mL, 1.53 mmol) at 0° C. The reaction was stirred for at room temperature for 2 hh, then concentrated under reduced pressure to afford 4-bromo-1,3-thiazole-2-carbonyl chloride (10.8 g, crude).

Step 4. A solution of ethyl 2-[(diphenylmethylidene)amino]acetate (12.75 g, 47.69 mmol) in THF (100 mL) at −78° C., was added LiHMDS (47.69 mL, 47.69 mmol), and stirred at −40° C. for 30 minutes. Then the reaction mixture was added a solution of 4-bromo-1,3-thiazole-2-carbonyl chloride (10.8 g, 47.69 mmol) in THF (100 mL) at −78° C. and stirred at room temperature for 12 hh. The resulting mixture was quenched with water (100 mL), extracted with EtOAc (100 mL×3). The combined organic layers were dried over anhydrous sodium sulfate, filtered and concentrated under reduced pressure to afford ethyl 3-(4-bromothiazol-2-yl)-2-((diphenylmethylene)amino)-3-oxopropanoate (27 g, crude) as a solid. LCMS (ESI): m/z: [M+H] calc'd for C₂₁H₁₇BrN₂O₃S 456.0; found 457.0.

Step 5. A solution of ethyl 3-(4-bromothiazol-2-yl)-2-((diphenylmethylene)amino)-3-oxopropanoate (20 g, 43.73 mmol) in THF (150 mL) at 0° C., was added 1 M HCl (100 mL) and stirred at room temperature for 2 hh. The resulting solution was concentrated and washed with ethyl ether (200 mL×2). The water phase was adjusted pH to 8 with sodium bicarbonate solution, then extracted with EtOAc (100 mL×3). The combined organic layers were dried over anhydrous sodium sulfate, filtered and concentrated under reduced pressure to afford ethyl 2-amino-3-(4-bromothiazol-2-yl)-3-oxopropanoate as an oil (9 g, crude). LCMS (ESI): m/z: [M+H] calc'd for C₈H₉BrN₂O₃S 292.0; found 292.9.

Step 6. A solution of ethyl 2-amino-3-(4-bromothiazol-2-yl)-3-oxopropanoate (10 g, 34.11 mmol) in MeOH (200 mL) at 0° C., was added benzaldehyde (7.24 g, 68.23 mmol), zinc chloride (9.3 g, 68.23 mmol) and NaBH₃CN (4.29 g, 68.23 mmol). The reaction was stirred at room temperature for 2 hh, then quenched with water (100 mL) and concentrated. The resulting mixture was extracted with EtOAc (100 mL×3). The combined organic layers were dried over anhydrous sodium sulfate, filtered and concentrated under reduced pressure. The residue was purified by silica gel chromatography to afford ethyl 3-(4-bromothiazol-2-yl)-2-(dibenzylamino)-3-oxopropanoate as a solid (8.4 g, 52% yield). LCMS (ESI): m/z: [M+H] calc'd for C₂₂H₂₁BrN₂O₃S 472.1; found 473.0.

Step 7. A mixture of ethyl 3-(4-bromothiazol-2-yl)-2-(dibenzylamino)-3-oxopropanoate (5 g, 10.56 mmol) and (R,R)-TS-DENEB (1.375 g, 2.11 mmol) in DCM (100 mL), was added HCOOH (1.99 mL, 43.29 mmol) and diethylamine (2.2 mL, 2.11 mmol) dropwise at room temperature under nitrogen atmosphere. The reaction mixture was stirred at 50° C. for 12 hh under nitrogen atmosphere. The resulting mixture was concentrated under reduced pressure. The residue was purified by silica gel column chromatography to afford ethyl (2S,3S)-3-(4-bromothiazol-2-yl)-2-(dibenzylamino)-3-hydroxypropanoate (3.148 g, 60% yield) as an oil. LCMS (ESI): m/z: [M+H] calc'd for C₂₂H₂₃BrN₂O₃S 474.1; found 475.0.

Step 8. A mixture of ethyl (2S,3S)-3-(4-bromothiazol-2-yl)-2-(dibenzylamino)-3-hydroxypropanoate (1 g, 2.1 mmol) and Ag₂O (4.88 g, 21.06 mmol) in acetonitrile (10 mL), was added iodomethane (3.58 g, 25.22 mmol) in portions. The reaction mixture was stirred at 50° C. for 12 hh, then filtered. The filter cake was washed with MeOH (50 mL×2). The filtrate was concentrated under reduced pressure to afford ethyl (2S,3S)-3-(4-bromothiazol-2-yl)-2-(dibenzylamino)-3-methoxypropanoate (1.06 g, crude) as an oil. LCMS (ESI): m/z: [M+H] calc'd for C₂₃H₂₅BrN₂O₃S 488.1; found 489.3.

Step 9. A mixture of ethyl (2S,3S)-3-(4-bromothiazol-2-yl)-2-(dibenzylamino)-3-hydroxypropanoate (1.06 g, 2.3 mmol) in HCl (10 ml, 8 M) was stirred at 80° C. for 12 hh and concentrated by reduced pressure. The residue was purified by reverse phase chromatography to afford (2S,3S)-3-(4-bromothiazol-2-yl)-2-(dibenzylamino)-3-methoxypropanoic acid (321 mg, 31.7% yield) as a solid. LCMS (ESI): m/z: [M+H] calc'd for C₂₁H₂₁BrN₂O₃S 460.1; found 461.1.

Step 10. A solution of (2S,3S)-3-(4-bromothiazol-2-yl)-2-(dibenzylamino)-3-methoxypropanoic acid (4.61 g, 10 mmol) in DCM (100 mL) at 0° C. was added methyl (3S)-1,2-diazinane-3-carboxylate bis(trifluoroacetic acid) salt (3.72 g, 15 mmol), NMM (10.1 mL. 100 mmol), EDCI (3.8 g, 20 mmol) and HOBt (5.39 g, 39.89 mmol). The solution was warmed to room temperature and stirred for 1 h. The reaction was then quenched with H₂O (100 mL) and was extracted with EtOAc (100 mL×3). The combined organic layers were dried over anhydrous sodium sulfate, filtered, and concentrated under reduced pressured. The residue was purified by silica gel column chromatography to give methyl (S)-1-((2S,3S)-3-(4-bromothiazol-2-yl)-2-(dibenzylamino)-3-methoxypropanoyl)hexahydropyridazine-3-carboxylate (5.11 g, 90% yield) as a solid. LCMS (ESI): m/z: [M+H] calc'd for C₂₇H₃₁BrN₄O₄S 587.1; found 586.1.

Step 11. A solution of methyl (S)-1-((2S,3S)-3-(4-bromothiazol-2-yl)-2-(dibenzylamino)-3-methoxypropanoyl)hexahydropyridazine-3-carboxylate (5.11 g, 9 mmol) in THF (100 mL)/H₂O (100 mL) was added LiOH (1.81 g, 45 mmol) under N₂ atmosphere and the resulting mixture was stirred for 2 hh at 25° C. The resulting mixture was concentrated under reduced pressure, the residue was acidified to pH 5 with HCL (1N). The aqueous layer was extracted with DCM (50 mL×3). The combined organic phase was concentrated under reduced pressure to give (S)-1-((2S,3S)-3-(4-bromothiazol-2-yl)-2-(dibenzylamino)-3-methoxypropanoyl)hexahydropyridazine-3-carboxylic acid (4.38 g, 85% yield) as a solid. LCMS (ESI): m/z [M+H] calc'd for C₂₆H₂₉BrN₄O₄S 572.1; found 573.1.

Step 12. A mixture of (S)-1-((2S,3S)-3-(4-bromothiazol-2-yl)-2-(dibenzylamino)-3-methoxypropanoyl)hexahydropyridazine-3-carboxylic acid (1.15 g, 2 mmol) and (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (985 mg, 2 mmol) in DCM (50 mL), was added DIEA (1.034 g, 8 mmol), EDCI (1.15 g, 558.3 mmol), HOBT (270.2 mg, 2 mmol). The reaction solution was stirred at 25° C. for 16 hh. The resulting mixture was diluted with DCM (200 mL), washed with water (50 mL×2) and brine (50 mL×3) and dried over anhydrous sodium sulfate, filtered and concentrated under reduced pressure. The residue was purified by silica gel column chromatography to afford 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (S)-1-((2S,3S)-3-(4-bromothiazol-2-yl)-2-(dibenzylamino)-3-methoxypropanoyl)hexahydropyridazine-3-carboxylate (1.13 g, 54% yield) as a solid. LCMS (ESI): m/z: [M+H] calc'd for C₅₅H₆₈BBrN₆O₇S 1046.4; found 1047.4.

Step 13. A mixture of 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (S)-1-((2S,3S)-3-(4-bromothiazol-2-yl)-2-(dibenzylamino)-3-methoxypropanoyl)hexahydropyridazine-3-carboxylate (250 mg, 0.24 mmol) and Pd(DtBPF)Cl₂ (15.55 mg, 0.024 mmol) in dioxane (5 mL) and water (1 mL), was added K₃PO₄ (126.59 mg, 0.6 mmol) in portions under nitrogen atmosphere. The reaction mixture was stirred at 80° C. for 2 hh under nitrogen atmosphere. The resulting mixture was diluted with water (20 mL) and extracted with EtOAc (10 mL×3), dried over anhydrous sodium sulfate, filtered and concentrated under reduced pressure. The residue was purified by silica gel column chromatography to afford (6³S,3S,4S,Z)-4-(dibenzylamino)-1¹-ethyl-3-methoxy-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (137 mg, 44.38%) as a solid. LCMS (ESI): m/z: [M+H] calc'd for C₄₉H₅₆N₆O₅S 840.4; found 841.5.

Step 14. A mixture of ((6³S,3S,4S,Z)-4-(dibenzylamino)-1¹-ethyl-3-methoxy-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (100 mg, 0.12 mmol) and Pd/C (253.06 mg, 2.38 mmol) in MeOH (10 mL), was added HCOONH₄ (149.94 mg, 2.38 mmol) in portions. The reaction mixture was stirred at 60° C. for 6 hh under hydrogen atmosphere. The resulting mixture was filtered, the filter cake was washed with MeOH (100 mL×10). The filtrate was concentrated under reduced pressure to afford (6³S,3S,4S,Z)-4-amino-1¹-ethyl-3-methoxy-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (56 mg, crude) as an oil. LCMS (ESI): m/z: [M+H] calc'd for C₃₅H₄₄N₆O₅S 660.3; found 661.2.

Step 15. A mixture of (6³S,3S,4S,Z)-4-amino-1¹-ethyl-3-methoxy-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (56 mg, 0.085 mmol) and N-(dimethylcarbamoyl)-N-methyl-L-valine (51.42 mg, 0.25 mmol) in DMF (2 mL), was added 2-Chloro-1,3-dimethylimidazolidinium hexafluorophosphate (47.55 mg, 0.17 mmol) and DIEA (547.62 mg, 4.24 mmol) in portions. The reaction mixture was stirred for 12 hh. The resulting mixture was purified by reverse phase chromatography to afford (2S)—N-((6³S,3S,4S,Z)-1¹-ethyl-3-methoxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-(1,3,3-trimethylureido)butanamide (1.5 mg, 2.06% yield) as a solid. ¹H NMR (400 MHz, Methanol-d4) δ 8.74-8.77 (m, 1H), 8.61 (d, J=1.6 Hz, 1H), 7.99-7.87 (m, 1H), 7.73-7.66 (m, 1H), 7.68 (s, 1H), 7.60-7.55 (m, 1H), 7.49 (d, J=8.7 Hz, 1H), 7.31 (d, J=51.0 Hz, 0H), 5.89 (s, 1H), 4.95 (s, 1H), 4.43 (d, J=13.0 Hz, 1H), 4.36 (q, J=6.2 Hz, 1H), 4.33-4.19 (m, 2H), 4.10-4.03 (m, 1H), 4.03 (d, J=11.2 Hz, 1H), 3.78-3.67 (m, 2H), 3.65 (s, OH), 3.46 (s, 3H), 3.34 (s, 4H), 3.01 (d, J=10.3 Hz, 1H), 2.93 (s, 6H), 2.88-2.81 (m, 1H), 2.78 (s, 3H), 2.70-2.60 (m, 1H), 2.23-2.01 (m, 2H), 2.03 (s, OH), 1.99 (d, J=13.3 Hz, 1H), 1.91-1.74 (m, 1H), 1.69-1.54 (m, 1H), 1.45 (d, J=6.2 Hz, 3H), 1.37-1.32 (m, 1H), 1.28 (s, 1H), 0.94 (p, J=6.7 Hz, 12H), 0.51 (s, 3H), 0.10 (s, 1H). LCMS (ESI): m/z: [M+H] calc'd for C₄₄H₆₀N₈O₇ 844.4; found 845.4.

### Example A286. The synthesis of (1S,2S)—N-((6³S,4S,Z)-1¹-ethyl-1²-(2-((S)-1-methoxyethyl)pyridin-3-yl)-6⁴,10,10-trimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

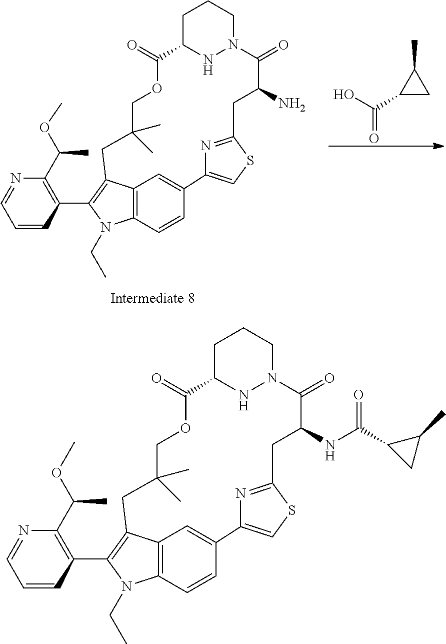

Step 1. A solution of Intermediate 8 (8 g, 10.95 mmol) in HCl (200 mL, 4M in 1,4-dioxane) was stirred at 0° C. for 2 hh, then concentrated under reduced pressure. The resulting mixture was diluted with DCM (60 mL) and saturated NaHCO₃ aqueous solution (40 mL). The organic phase was separated and washed with brine (50 mL×2) and concentrated under reduced pressure to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (10.3 g, crude) as solid. LCMS (ESI): m/z: [M+H] calc'd for C₃₄H₄₂N₆O₄S 630.3; found 631.2.

Step 2. A stirred solution of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (8 g, 12.68 mmol) in DMF (50 mL) at 0° C., was added DIEA (9.83 g, 76.09 mmol), (1 S,2S)-2-methylcyclopropane-1-carboxylic acid (1.52 g, 15.22 mmol) and HATU (14.47 g, 38.05 mmol). The reaction mixture was stirred at 0° C. for 2 hh and concentrated under reduced pressure. The residue was purified by reverse phase chromatography to afford (1S,2S)—N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide (6.84 g, 56.37% yield) as a solid. ¹H NMR (400 MHz, DMSO-d₆) δ 8.79 (dd, J=4.7, 1.9 Hz, 1H), 8.59-8.40 (m, 2H), 7.95-7.86 (m, 1H), 7.82-7.71 (m, 2H), 7.66-7.53 (m, 2H), 5.57 (t, J=9.0 Hz, 1H), 5.07 (s, 1H), 4.41-4.28 (m, 2H), 4.25 (d, J=12.4 Hz, 1H), 4.17 (d, J=10.8 Hz, 1H), 4.09 (d, J=7.2 Hz, 1H), 3.58 (s, 2H), 3.32 (d, J=14.6 Hz, 1H), 3.28 (s, 3H), 3.16 (dd, J=14.7, 9.1 Hz, 1H), 2.95 (d, J=14.4 Hz, 1H), 2.75 (m, J=12.1, 7.1 Hz, 1H), 2.43 (d, J=14.4 Hz, 1H), 2.13-2.00 (m, 1H), 1.76 (d, J=22.0 Hz, 2H), 1.60-1.44 (m, 2H), 1.38 (d, J=6.1 Hz, 3H), 1.07 (d, J=1.9 Hz, 4H), 0.86 (dd, J=14.1, 7.1 Hz, 7H), 0.59-0.49 (m, 1H), 0.34 (s, 3H). LCMS (ESI): m/z: [M+H] calc'd for C₃₉H₄₆N₆O₅S 712.3; found 713.2.

### Example A613. The synthesis of N-(2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyrndina-6(1,3)-pyridazinacycloundecaphane-4-yl)amino)-3-methyl-1-oxobutan-2-yl)-3-methoxy-N-methylazetidine-1-carboxamide

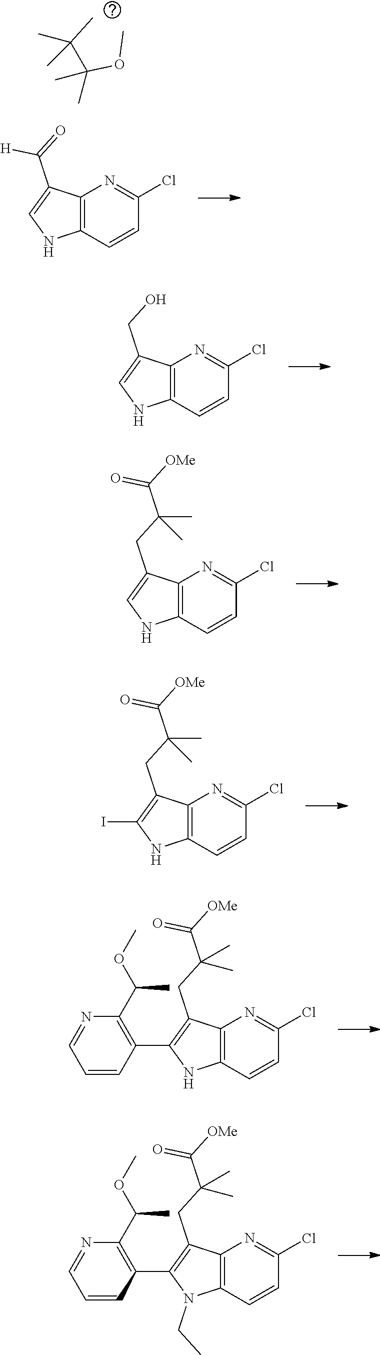

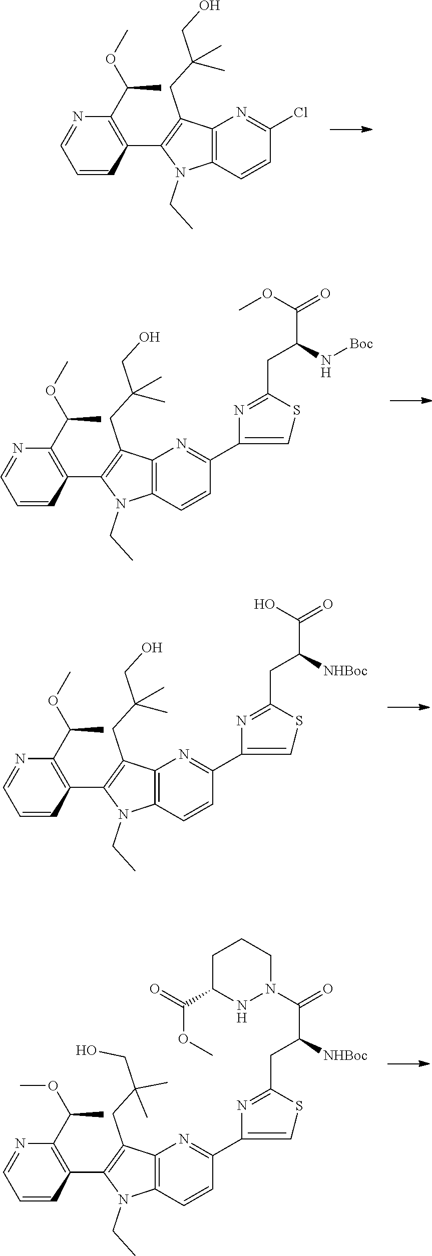

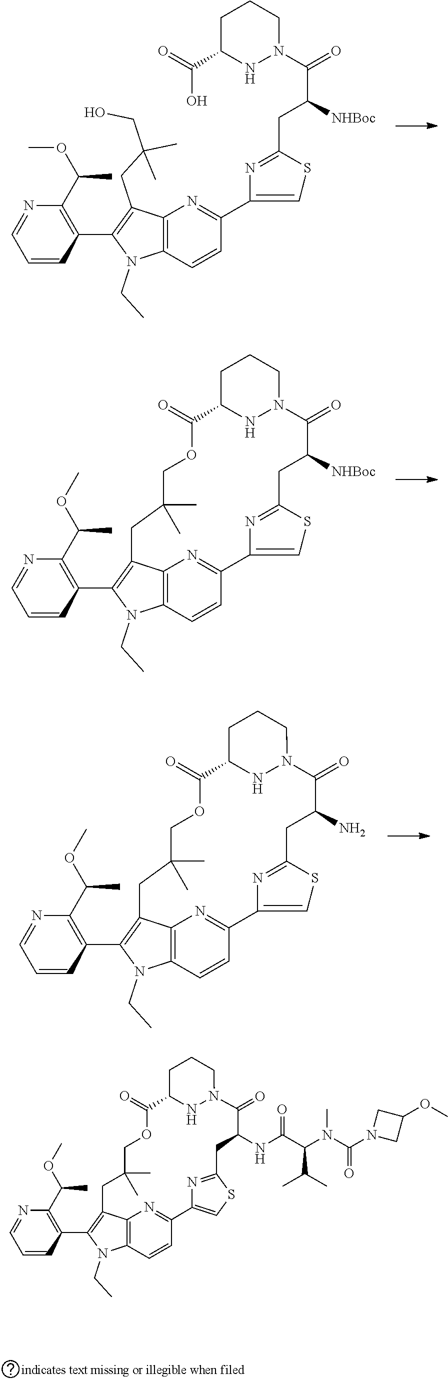

Step 1. A mixture of methyl (S)-3-(4-bromothiazol-2-yl)-2-((tert-butoxycarbonyl)amino)propanoate (920 mg, 2.5 mmol), 4,4,5,5-tetramethyl-2-(4,4,5,5-tetramethyl-1,3,2-dioxaborolan-2-yl)-1,3,2-dioxaborolane (1.6 g, 6.3 mmol), x-Phos (180 mg, 0.5 mmol), Pd₂(dba)₃-chloroform (130 mg, 0.13 mmol) and potassium acetate (740 mg, 7.5 mmol) in dioxane (25 mL) in a sealed tube under N₂ atmosphere, was stirred at 110° C. for 8 hh to afford crude methyl (S)-2-((tert-butoxycarbonyl)amino)-3-(4-(4,4,5,5-tetramethyl-1,3,2-dioxaborolan-2-yl)thiazol-2-yl)propanoate as a solution. LCMS (ESI): m/z [M+H] calc'd for C₁₈H₂₉BN₂O₆S 412.2; found 331.1.

Step 2. A mixture of 5-chloro-1H-pyrrolo[3,2-b]pyridine-3-carbaldehyde (7 g, 39 mmol) in MeOH (140 mL) under N₂ atmosphere, was added NaBH₄ (2.9 g, 78 mmol) at 0° C. The reaction mixture was stirred at 10° C. for 2 hh and concentrated under reduced pressure. The residue was diluted with EtOAc (200 mL), washed with brine (25 mL), dried over anhydrous sodium sulfate and concentrated under reduced pressure. The residue was purified by silica gel column chromatography to afford (5-chloro-1H-pyrrolo[3,2-b]pyridin-3-yl)methanol (3.5 g, 55% yield) as a solid. LCMS (ESI): m/z: [M+H] calc'd for C₈H₇ClN₂O 182.0; found 183.0.

Step 3. A mixture of (5-chloro-1H-pyrrolo[3,2-b]pyridin-3-yl)methanol (3.5 g, 19 mmol) and ((1-methoxy-2-methylprop-1-en-1-yl)oxy)trimethylsilane (6.7 g, 38 mmol) in THF (50 mL), was dropwise added TMSOTf (3.8 g, 17.1 mmol) at 0° C. The reaction mixture was stirred at 5° C. for 2 hh, then diluted with EtOAc (100 mL), washed with saturated NaHCO₃ aqueous (50 mL), and brine (50 mL×2). The organic layer was dried over anhydrous sodium sulfate and concentrated under reduced pressure. The residue was purified by silica gel column chromatography to afford methyl 3-(5-chloro-1H-pyrrolo[3,2-b]pyridin-3-yl)-2,2-dimethylpropanoate (3 g, 59% yield) as a solid. LCMS (ESI): m/z: [M+H] calc'd for C₁₃H₁₅ClN₂O₂ 266.1; found 267.1.

Step 4. A mixture of methyl 3-(5-chloro-1H-pyrrolo[3,2-b]pyridin-3-yl)-2,2-dimethylpropanoate (3 g, 11 mmol) in anhydrous THF (50 mL) at 0° C., was added AgOTf (4.3 g, 17 mmol) and 12 (2.9 g, 11 mmol). The reaction mixture was stirred at 0° C. for 2 hh, then quench with conc. Na₂SO₃ (20 mL), diluted with EtOAc (50 mL) and filtered. The filtrate was washed with brine (50 mL). The organic layer was dried over anhydrous sodium sulfate and concentrated under reduced pressure. The residue was purified with silica gel column chromatography to afford methyl 3-(5-chloro-2-iodo-1H-pyrrolo[3,2-b]pyridin-3-yl)-2,2-dimethylpropanoate (2.3 g, 52% yield) as a solid. LCMS (ESI): m/z: [M+H] calc'd for C₁₃H₁₄ClIN₂O₂ 393.0; found 392.0.

Step 5. A mixture of methyl 3-(5-chloro-2-iodo-1H-pyrrolo[3,2-b]pyridin-3-yl)-2,2-dimethylpropanoate (2.3 g, 5.9 mmol), 2-(2-(2-methoxyethyl)phenyl)-4,4,5,5-tetramethyl-1,3,2-dioxaborolane (1.6 g, 7.1 mmol) and K₂CO₃ (2.4 g, 18 mol) in dioxane (25 mL) and water (5 mL) under N₂ atmosphere, was added Pd(dppf)Cl₂-DCM (480 mg, 0.59 mmol). The reaction mixture was stirred at 70° C. for 4 hh, then diluted with EtOAc (200 mL) and washed with brine (25 mL). The separated organic layer was dried over anhydrous sodium sulfate and concentrated under reduced pressure. The residue was purified by silica gel column chromatography to afford methyl (S)-3-(5-chloro-2-(2-(1-methoxyethyl)pyridin-3-yl)-1H-pyrrolo[3,2-b]pyridin-3-yl)-2,2-dimethylpropanoate (2 g, yield 84%) as a solid. LCMS (ESI): m/z [M+H] calc'd for C₂₁H₂₄ClN₃O₃ 401.2; found 402.2.

Step 6. A mixture of methyl (S)-3-(5-chloro-2-(2-(1-methoxyethyl)pyridin-3-yl)-1H-pyrrolo[3,2-b]pyridin-3-yl)-2,2-dimethylpropanoate (2 g, 5 mmol), cesium carbonate (3.3 g, 10 mmol) and EtI (1.6 g, 10 mmol) in DMF (30 mL) was stirred at 25° C. for 10 hh. The resulting mixture was diluted with EtOAc (100 mL), washed with brine (20 mL×4). The separated organic layer was dried over anhydrous sodium sulfate, filtered and concentrated under reduced pressure. The residue was purified by silica gel column chromatography to afford methyl (S)-3-(5-chloro-1-ethyl-2-(2-(1-methoxyethyl)pyridin-3-yl)-1H-pyrrolo[3,2-b]pyridin-3-yl)-2,2-dimethylpropanoate as two diastereomers (P1: 0.7 g, 32% yield; P2: 0.6 g, 28% yield) both as a solid. LCMS (ESI): m/z: [M+H] calc'd for C₂₃H₂₈ClN₃O₃ 429.2; found 430.2.

Step 7. A mixture of methyl (S)-3-(5-chloro-1-ethyl-2-(2-(1-methoxyethyl)pyridin-3-yl)-1H-pyrrolo[3,2-b]pyridin-3-yl)-2,2-dimethylpropanoate (P2, 1.2 g, 2.8 mmol) in anhydrous THF (20 mL) at 5° C., was added LiBH₄ (120 mg, 5.6 mmol). The reaction mixture was stirred at 60° C. for 4 hh, then quenched with conc. NH₄Cl (20 mL), diluted with EtOAc (50 mL) and washed with brine (30 mL). The organic layer was separated, dried over anhydrous sodium sulfate and concentrated under reduced pressure. The residue was purified with silica gel column chromatography to afford (S)-3-(5-chloro-1-ethyl-2-(2-(1-methoxyethyl)pyridin-3-yl)-1H-pyrrolo[3,2-b]pyridin-3-yl)-2,2-dimethylpropan-1-ol (1 g, 89% yield) as a solid. LCMS (ESI): m/z: [M+H] calc'd for C₂₂H₂₈ClN₃O₂ 401.2; found 402.2.

Step 8. A mixture of solution from Step 1 (360 mg, crude, 1 mmol) in dioxane (10 mL) and water (2 mL), was added (S)-3-(5-chloro-1-ethyl-2-(2-(1-methoxyethyl)pyridin-3-yl)-1H-pyrrolo[3,2-b]pyridin-3-yl)-2,2-dimethylpropan-1-ol (200 mg, 0.5 mmol), potassium carbonate (200 mg, 1.5 mmol) and Pd-118 (30 mg, 0.05 mmol). This reaction mixture was stirred at 70° C. for 3 hh, then diluted with EtOAc (40 mL), filtered. The filtrate was washed with brine, dried over anhydrous sodium sulfate, filtered and concentrated under reduced pressure. The residue was purified with silica gel column chromatography to afford methyl (S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoate (300 mg, 65% yield) as a solid. LCMS (ESI): m/z: [M+H] calc'd for C₃₄H₄₅N₅O₆S 651.3; found 652.3.

Step 9. A solution of methyl (S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoate (280 mg, 0.43 mmol) in MeOH (4 mL), was added a solution of lithium hydroxide (51 mg, 2.15 mmol) in water (2 mL) at 20° C. The reaction was stirred at 20° C. for 5 hh, then adjusted to pH=3-4 with HCl (1 N). The resulting mixture was diluted with water (30 mL) and extracted with EtOAc (15 mL×3). The combined organic phase was washed with brine (10 mL), dried over anhydrous sodium sulfate, filtered and concentrated under reduced pressure to give (S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoic acid (280 mg, crude) as a solid. LCMS (ESI): m/z: [M+H] calc'd for C₃₃H₄₃N₅O₆S 637.3; found 638.3.

Step 10. A solution of (S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoic acid (274 mg, 0.43 mmol) and methyl (S)-hexahydropyridazine-3-carboxylate (280 mg, 0.64 mmol) in DMF (3 mL) at 5° C., was added a solution of HATU (245 mg, 0.64 mmol) and DIEA (555 mg, 4.3 mmol) in DMF (2 mL). The reaction was stirred for 1 h, then diluted with EtOAc (20 mL) and water (20 mL). The organic layer was separated and washed with water (20 mL×3) and brine (20 mL), dried over anhydrous sodium sulfate, filtered concentrated under reduced pressure. The residue was purified by silica gel chromatography to give methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoyl)hexahydropyridazine-3-carboxylate (230 mg, 70% yield) as solid. LCMS (ESI): m/z: [M+H] calc'd for C₃₉H₅₃N₇O₇S 763.4; found 764.3.

Step 11. A solution of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoyl)hexahydropyridazine-3-carboxylate (230 mg, 0.3 mmol) in DCE (3 mL), was added trimethyltin hydroxide (300 mg, 1.4 mmol) under N₂ atmosphere. The reaction was stirred at 65° C. for 16 hh, then concentrated under reduced pressure. The residue was diluted with EtOAc (20 mL), washed with water (20 mL) and brine (10 mL), dried over anhydrous sodium sulfate, filtered and concentrated under reduced pressure to afford (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoyl)hexahydropyridazine-3-carboxylic acid (200 mg, crude) as foam. LCMS (ESI): m/z: [M+H] calc'd for C₃₈H₅₁N₇O₇S 749.4; found 750.3.

Step 12. A solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-pyrrolo[3,2-b]pyridin-5-yl)thiazol-2-yl)propanoyl)hexahydropyridazine-3-carboxylic acid (245 mg, 0.32 mmol) in DCM (50 mL) at 5° C., were added HOBt (432 mg, 3.2 mmol), EDCI (1.8 g, 9.6 mmol) and DIEA (1.65 g, 12.8 mmol). The reaction mixture was stirred at 20° C. for 16 hh, then concentrated under reduced pressure. The residue was diluted with EtOAc (20 mL) and water (20 mL). The organic layer was separated and washed with water (30 mL×3) and brine (30 mL), dried over anhydrous sodium sulfate, filtered and concentrated under reduced pressure. The residue was purified by silica gel chromatography to give tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-4-yl)carbamate (100 mg, 43% yield) as solid. LCMS (ESI): m/z: [M+H] calc'd for C₃₈H₄₉N₇O₆S 731.4; found 732.3.

Step 13. A solution of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-4-yl)carbamate (80 mg, 0.11 mmol) in TFA (0.2 mL) and DCM (0.6 mL) was stirred at 20° C. for 1 h. The reaction was concentrated to afford (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-5,7-dione (72 mg, 95% yield) as a solid. LCMS (ESI): m/z: [M+H] calc'd for C₃₃H₄₁N₇O₄S 631.3; found 632.3.

Step 14. A solution of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-pyrrolo[3,2-b]pyridina-6(1,3)- pyridazinacycloundecaphane-5,7-dione (100 mg, 0.16 mmol) and (2S)-2-[(3-methoxyazetidin-1-yl)carbonyl(methyl)amino]-3-methylbutanoic acid (78 mg, 0.32 mmol) in DMF (5 mL) at 0° C., was dropwise added a solution of DIEA (620 mg, 4.8 mmol) and HATU (91 mg, 0.24 mmol) in DMF (5 mL). The reaction mixture was stirred at 0° C. for 2 hh, then diluted with EtOAc (50 mL), washed with water (25 mL×3), dried over anhydrous sodium sulfate, filtered and concentrated under reduced pressure. The residue was purified by silica gel column chromatography to afford N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-pyrrolo[3,2-b]pyridina-6(1,3)-pyridazinacycloundecaphane-4-yl)amino)-3-methyl-1-oxobutan-2-yl)-3-methoxy-N-methylazetidine-1-carboxamide (112.9 mg, 82% yield) as a solid. ¹H NMR (400 MHz, CD₃OD) δ 8.77-8.75 (dd, J=4.8, 1.7 Hz, 1H), 7.96-7.94 (d, J=8.6 Hz, 1H), 7.89-7.87 (dd, J=8.4, 2.3 Hz, 2H), 7.77-7.74 (d, J=8.6 Hz, 1H), 7.58-7.55 (dd, J=7.8, 4.8 Hz, 1H), 5.73-5.70 (dd, J=8.0, 2.7 Hz, 1H), 4.41-4.38 (dt, J=8.5, 4.3 Hz, 2H), 4.33-4.26 (m, 3H), 4.24-4.17 (m, 3H), 4.04-4.01 (dd, J=11.9, 3.0 Hz, 1H), 3.99-3.96 (m, 1H), 3.89-3.83 (m, 2H), 3.53-3.49 (dd, J=9.7, 7.3 Hz, 2H), 3.46-3.45 (d, J=3.0 Hz, 1H), 3.35 (s, 3H), 3.34-3.33 (d, J=4.5 Hz, 3H), 3.28 (s, 1H), 2.89 (s, 3H), 2.78-2.71 (td, J=13.2, 3.4 Hz, 1H), 2.52-2.48 (d, J=14.1 Hz, 1H), 2.23-2.20 (m, 1H), 2.19-2.11 (d, J=10.2 Hz, 1H), 1.91-1.88 (d, J=13.5 Hz, 1H), 1.73-1.70 (dd, J=9.0, 3.9 Hz, 1H), 1.56-1.50 (m, 1H), 1.47-1.46 (d, J=6.1 Hz, 3H), 0.98-0.91 (m, 9H), 0.88 (s, 3H), 0.45 (s, 3H). LCMS (ESI): m/z: [M+H] calc'd for C₄₄H₅₉N₉O₇S 857.4; found 858.3.

### Example A579. The synthesis of N-((2S)-1-(((6³S,6⁴S,4S,Z)-1¹-ethyl-1²-(2-((S)-1-methoxyethyl)pyridin-3-yl)-6⁴,10,10-trimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-3-methoxy-N-methylazetidine-1-carboxamide

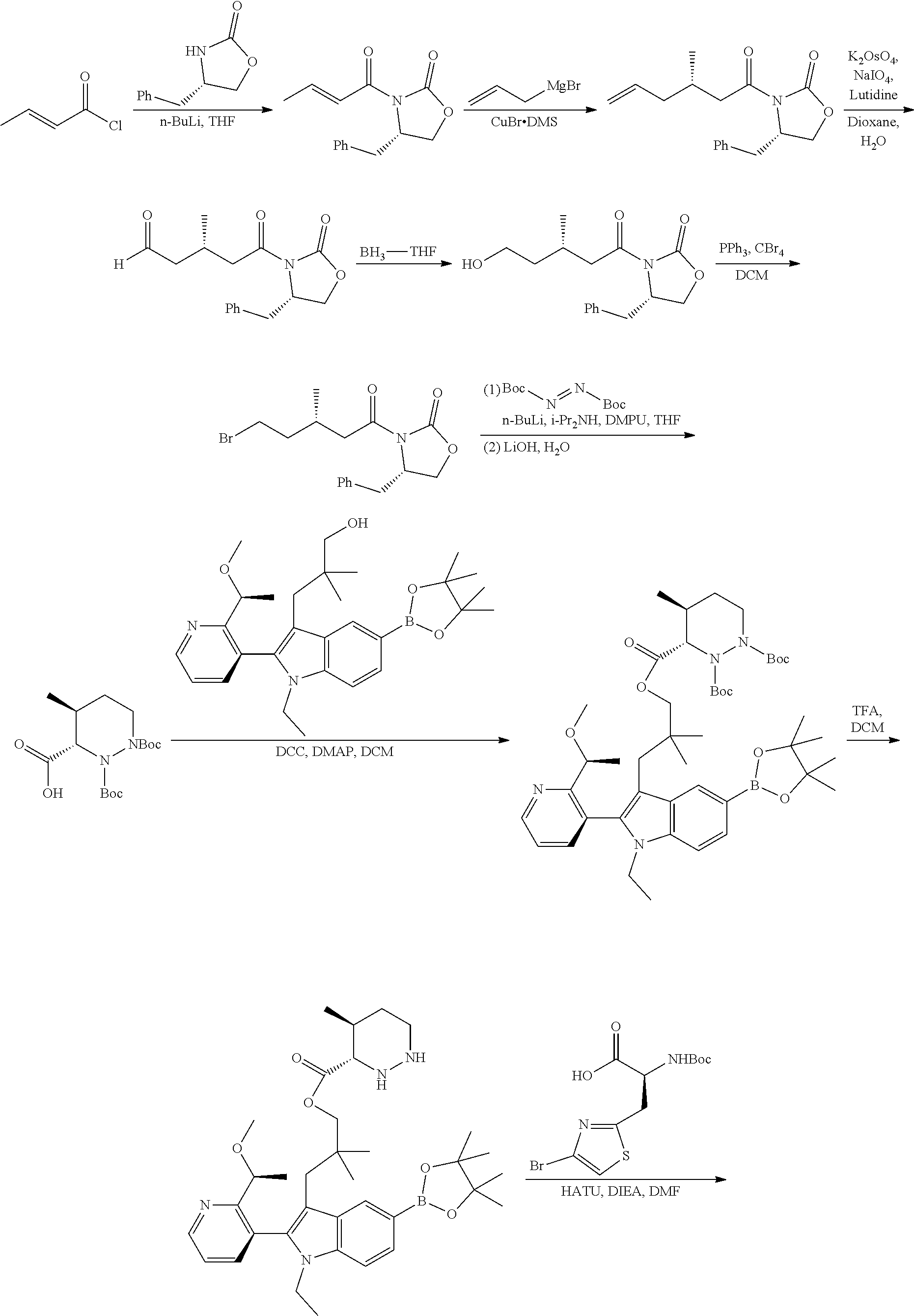

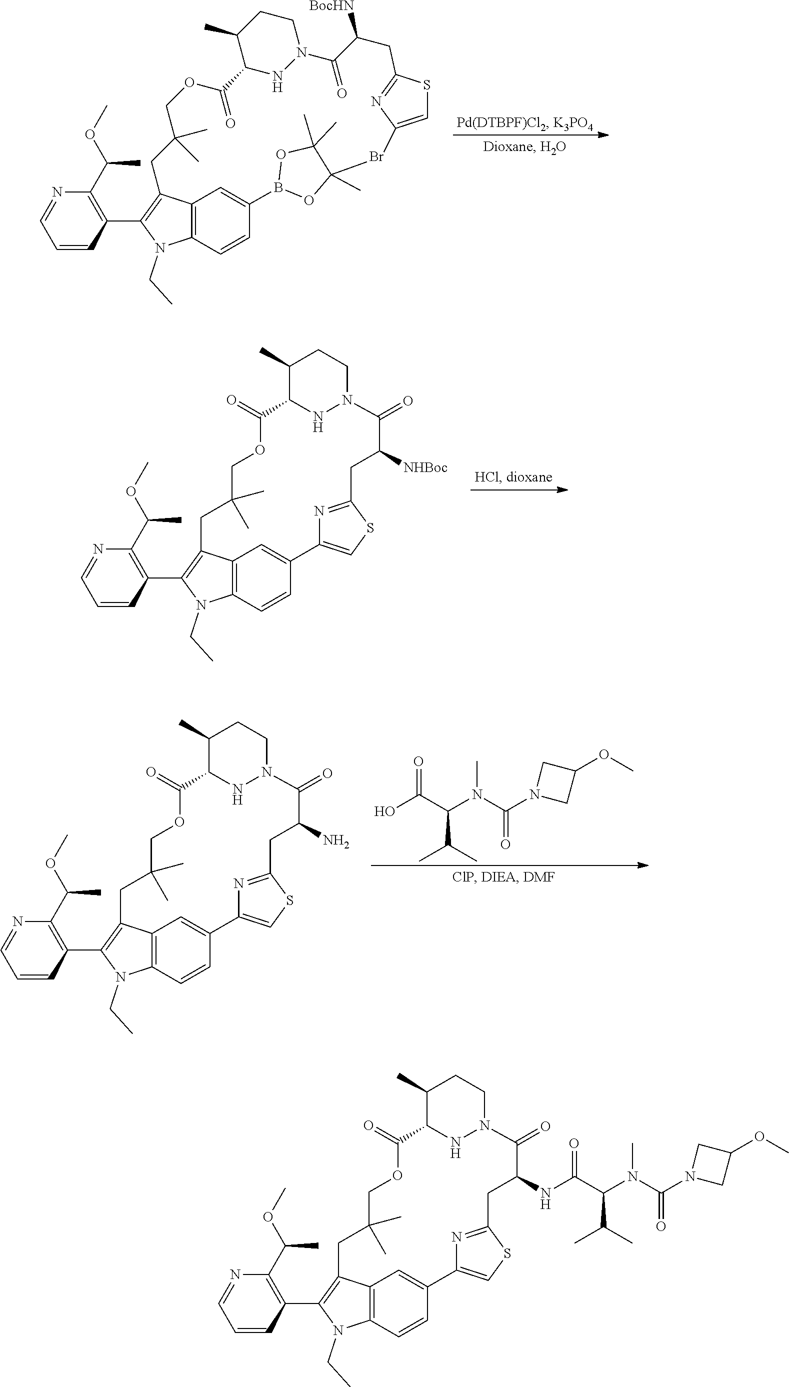

Step 1. A solution of (S)-4-benzyloxazolidin-2-one (10 g, 56.43 mmol) in THF (100 mL) was purged with nitrogen, was added of n-butyllithium (24.83 mL, 62.08 mmol) at −78° C. under nitrogen atmosphere, then stirred for at −78° C. for 15 minutes. The reaction mixture was added 2-butenoyl chloride (6.49 g, 62.08 mmol). The resulting solution was stirred at −78° C. for 30 minutes, then slowly warmed up to 0° C. and stirred for 15 minutes, quenched with saturated ammonium chloride solution (100 mL). The resulting solution was extracted with EtOAc (100 mL×3) and the combined organic phase was dried over anhydrous sodium sulfate and concentrated under reduced pressure. The residue was purified by silica gel column chromatography to afford (4S)-4-benzyl-3-[(2E)-but-2-enoyl]-1,3-oxazolidin-2-one (12.26 g, 88.57% yield) as an oil. LCMS (ESI): m/z: [M+H] calc'd for C₁₄H₁₅NO₃ 245.1; found 246.1.

Step 2. A solution of CuBr·DMS (12.07 g, 58.71 mmol) in THF (120 mL) was purged and maintained nitrogen atmosphere, added of allylmagnesium bromide (58.71 mL, 58.71 mmol) at −78° C. The reaction was stirred at −60° C. for 30 minutes under nitrogen atmosphere followed by addition of (4S)-4-benzyl-3-[(2E)-but-2-enoyl]-1,3-oxazolidin-2-one (12 g, 48.92 mmol) at −78° C. The resulting solution was stirred at −50° C. for 3 more hh, then quenched with saturated ammonium chloride solution (100 mL) and extracted with EtOAc (60 mL×3). The combined organic phase was dried over anhydrous sodium sulfate and concentrated under reduced pressure. The residue was purified by silica gel column chromatography to afford (S)-4-benzyl-3-((S)-3-methylhex-5-enoyl)oxazolidin-2-one (13.2 g, 93.89% yield) as an oil. LCMS (ESI): m/z [M+H] calc'd for C₁₇H₂₁NO₃ 287.2; found 288.2.

Step 3. A solution of (S)-4-benzyl-3-((S)-3-methylhex-5-enoyl)oxazolidin-2-one (13.2 g, 45.94 mmol) in dioxane (200 ml) and water (200 mL), was added 2,4-Lutidine (9.84 g, 91.87 mmol) followed with K₂OsO₄·2H₂O (1.69 g, 4.59 mmol) at 0° C. The reaction solution was stirred at 0° C. for 15 minutes, then was added NaIO₄ (39.3 g, 183.74 mmol). The resulting mixture was stirred at 0° C. for 1 h, then extracted with EtOAA (150 mL×3). The combined organic phase was hydrochloric acid (100 mL×3), dried over anhydrous sodium sulfate and concentrated under reduced pressure to afford (S)-5-((S)-4-benzyl-2-oxooxazolidin-3-yl)-3-methyl-5-oxopentanal (12.3 g, crude) as an oil. LCMS (ESI): m/z: [M+H] calc'd for C₁₆H₁₉NO₄ 289.1; found 290.1.

Step 4. A solution of (S)-5-((S)-4-benzyl-2-oxooxazolidin-3-yl)-3-methyl-5-oxopentanal (12.3 g, 42.51 mmol) in THF (200 mL) was purged and maintained with nitrogen atmosphere, then added borane-tetrahydrofuran complex (55.27 mL, 55.27 mmol) at 0° C. The reaction was stirred at 0° C. for 30 minutes, then quenched with methanol (40 mL). The resulting mixture was concentrated under reduced pressure. The residue was purified by silica gel column chromatography to afford (S)-4-benzyl-3-((S)-5-hydroxy-3-methylpentanoyl)oxazolidin-2-one (9.6 g, 77.51% yield) as an oil. LCMS (ESI): m/z: [M+H] calc'd for C₁₆H₂₁NO₄ 291.1; found 292.1.

Step 5. A solution of (S)-4-benzyl-3-((S)-5-hydroxy-3-methylpentanoyl)oxazolidin-2-one (9.6 g, 32.95 mmol) and CBr₄ (16.39 g, 49.43 mmol) in DCM (120 mL) at 0° C., was added triphenylphosphine (12.96 g, 49.41 mmol). The reaction was stirred at 0° C. for 1 h, then quenched with ice water (100 mL) and extracted with DCM (100 mL×3). The combined organic phase was dried over anhydrous sodium sulfate and concentrated under reduced pressure. The residue was purified by silica gel column chromatography to afford (S)-4-benzyl-3-((R)-5-bromo-3-methylpentanoyl)oxazolidin-2-one (10 g, 85.67% yield) as an oil. LCMS (ESI): m/z: [M+H] calc'd for C₁₆H₂₀BrNO₄ 353.1; found 354.1.

Step 6. A mixture of n-BuLi (2.26 mL, 5.65 mmol) and diisopropylamine (571.3 mg, 5.65 mmol) in THF (10 mL) under nitrogen at −78° C., was added a cooled (−78° C.) solution of (S)-4-benzyl-3-((R)-5-bromo-3-methylpentanoyl)oxazolidin-2-one (2 g, 5.65 mmol) in THF (9 mL). The reaction mixture was stirred at −78° C. for 30 minutes, then was added a solution of (E)-N-[(tert-butoxycarbonyl)imino](tert-butoxy)formamide (1.3 g, 5.65 mmol) in THF (10 mL), stirred for another 30 minutes at −78° C. The resulting mixture was added DMPU (16 mL, 132.82 mmol) and warmed up to 0° C. and stirred for 90 minutes, followed by addition of a solution of LiOH·H₂O (1.18 g, 28.12 mmol) in water (20 mL). Then THF was removed under reduced pressure. The residue was washed with DCM (80 mL×3). The aqueous phase was acidified to pH 5-6 with HCl (aq.), extracted with mixture of DCM/methanol (80 mL×3, 10:1). The combined organic layers were dried over anhydrous sodium sulfate, filtered, concentrated under reduced pressure. The residue was purified by reverse phase chromatography to afford (3S,4S)-1,2-bis(tert-butoxycarbonyl)-4-methylhexahydropyridazine-3-carboxylic acid (296 mg, 15.22% yield) as a solid. LCMS (ESI): m/z: [M−H] calc'd for C₁₆H₂₈N₂O₆ 344.2; found 343.1.

Step 7. A mixture of (3S,4S)-1,2-bis(tert-butoxycarbonyl)-4-methylhexahydropyridazine-3-carboxylic acid (289 mg, 0.84 mmol) and (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (413.24 mg, 0.84 mmol) in DMF (10 mL) at 0° C., was added DMAP (51.26 mg, 0.42 mmol) and DCC (692.53 mg, 3.36 mmol). The reaction solution was stirred at room temperature for 1 h, then quenched with water/ice (10 mL), extracted with EtOAc (15 mL×3). The combined organic layers were washed with brine (50 mL×3), dried over anhydrous sodium sulfate and concentrated under reduced pressure. The residue was purified by silica gel column chromatography to afford 1,2-di-tert-butyl 3-(3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl) (3S,4S)-4-methyltetrahydropyridazine-1,2,3-tricarboxylate (538 mg, 78.3% yield) as a solid. LCMS (ESI): m/z: [M−H] calc'd for C₄₅H₆₇BN₄O₉ 818.5; found 819.4.

Step 8. A solution of 1,2-di-tert-butyl 3-(3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl) (3S,4S)-4-methyltetrahydropyridazine-1,2,3-tricarboxylate (508 mg, 0.62 mmol) in DCM (25 mL), was added TFA (25 mL) at 0° C. The reaction solution was stirred at room temperature for 1 h. The resulting mixture was concentrated to afford 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (3S,4S)-4-methylhexahydropyridazine-3-carboxylate (508 mg, crude) as an oil. LCMS (ESI): m/z: [M−H] calc'd for C₃₅H₅₁BN₄O₅ 618.4; found 619.3.

Step 9. A solution of 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (3S,4S)-4-methylhexahydropyridazine-3-carboxylate (508 mg, 0.82 mmol) and (S)-3-(4-bromothiazol-2-yl)-2-((tert-butoxycarbonyl)amino)propanoic acid (288.41 mg, 0.82 mmol) in DMF (50 mL) at 0° C., was added DIEA (1061.31 mg, 8.21 mmol), HATU (468.35 mg, 1.23 mmol). The reaction solution was stirred at room temperature for 1 h, then quenched with ice water (30 mL) and extracted with EtOAc (30 mL×3). The combined organic phase was washed with brine (50 mL×3), dried over anhydrous sodium sulfate and concentrated under reduced pressure. The residue was purified by silica gel column chromatography to afford 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (3S,4S)-1-((S)-3-(4-bromothiazol-2-yl)-2-((tert-butoxycarbonyl)amino)propanoyl)-4-methylhexahydropyridazine-3-carboxylate (431 mg, 55.14% yield) as a solid. LCMS (ESI): m/z: [M−H] calc'd for C₄₆H₆₄BBrN₆O₈S 950.4; found 951.3.

Step 10. A mixture of Pd(DTBpf)Cl₂ (27.39 mg, 0.042 mmol) and K₃PO₄ (89.2 mg, 0.42 mmol) in dioxane (5 mL) and water (1 mL) was purged nitrogen, stirred at 60° C. for 5 minutes under nitrogen atmosphere, then added a solution of 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (3S,4S)-1-((S)-3-(4-bromothiazol-2-yl)-2-((tert-butoxycarbonyl)amino)propanoyl)-4-methylhexahydropyridazine-3-carboxylate (200 mg, 0.21 mmol) in dioxane (5 mL) and water (1 mL) at 60° C. The reaction mixture was stirred at 60° C. for 1 h, then quenched with ice water (5 mL), extracted with EtOAc (15 mL×3). The combined organic layers were dried over anhydrous sodium sulfate and concentrated under reduced pressure. The residue was purified by silica gel column chromatography to afford tert-butyl ((6³S,6⁴S,4S,Z)-1¹-ethyl-1²-(2-((S)-1-methoxyethyl)pyridin-3-yl)-6⁴,10,10-trimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (70 mg, 44.72% yield) as a solid. LCMS (ESI): m/z: [M−H] calc'd for C₄₀H₅₂N₆O₆S 744.4; found 745.4.

Step 11. A solution of tert-butyl ((6³S,6⁴S,4S,Z)-1¹-ethyl-1²-(2-((S)-1-methoxyethyl)pyridin-3-yl)-6⁴,10,10-trimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate (70 mg, 0.094 mmol) in dioxane (5 mL), was added HCl in dioxane (5 mL, 4M). The reaction was stirred at room temperature for 1 h, then concentrated under reduced pressure to afford (6³S,6⁴S,4S,Z)-4-amino-1¹-ethyl-1²-(2-((S)-1-methoxyethyl)pyridin-3-yl)-6⁴,10,10-trimethyl-6¹,6²,6³,6⁴,6⁵,6⁶-hexahydro-1¹H-8-oxa-2(4,2)-thiazola-1(5,3)-indola-6(1,3)-pyridazinacycloundecaphane- 5,7-dione (124 mg, crude) as an oil. LCMS (ESI): m/z: [M−H] calc'd for C₃₆H₄₅N₅O₄S 644.3; found 645.3.

Step 12. A mixture of (6³S,6⁴S,4S,Z)-4-amino-1¹-ethyl-1²-(2-((S)-1-methoxyethyl)pyridin-3-yl)-6⁴,10,10-trimethyl-6¹,6²,6³,6⁴,6⁵,6⁶-hexahydro-1¹H-8-oxa-2(4,2)-thiazola-1(5,3)-indola-6(1,3)-pyridazinacycloundecaphane- 5,7-dione (112 mg, 0.17 mmol) and N-(3-methoxyazetidine-1-carbonyl)-N-methyl-L-valine (50.92 mg, 0.21 mmol) in DMF (3 ml) at 0 LC, was added DIEA (1.795 g, 13.9 mmol), 2-chloro-1,3-dimethylimidazolidinium hexafluorophosphate (72.57 mg, 0.26 mmol). The reaction was stirred at room temperature for 1 h and then filtered. The filtrate was purified by reverse phase chromatography to afford N-((2S)-1-(((6³S,6⁴S,4S,Z)-1¹-ethyl-1²-(2-((S)-1-methoxyethyl)pyridin-3-yl)-6⁴,10,10-trimethyl-5,7-dioxo-6¹,6²,6³,6⁴ 6⁵,6⁶-hexahydro-1¹H-8-oxa-2(4,2)-thiazola-1(5,3)-indola-6(1,3)-pynidazinacycloundecaphane-4-yl)amino)-3-methyl-1-oxobutan-2-yl)-3-methoxy-N-methylazetidine-1-carboxamide (25.6 mg, 16.92% yield) as a solid. ¹H NMR (400 MHz, DMSO-d₆) δ 8.76 (dd, J=4.7, 1.8 Hz, 1H), 8.60 (s, 1H), 8.30-8.20 (m, 1H), 7.86-7.70 (m, 3H), 7.61-7.50 (m, 2H), 5.57-5.43 (m, 1H), 5.07 (d, J=12.1 Hz, 1H), 4.39-4.21 (m, 3H), 4.20-4.01 (m, 5H), 3.96 (d, J=11.1 Hz, 1H), 3.82 (dd, J=8.9, 3.6 Hz, 1H), 3.77-3.71 (m, 1H), 3.63-3.55 (m, 2H), 3.35-3.27 (m, 2H), 3.24 (s, 3H), 3.23-3.14 (m, 4H), 2.93-2.79 (m, 2H), 2.70 (s, 3H), 2.15-2.01 (m, 1H), 1.83-1.61 (m, 2H), 1.38 (d, J=6.1 Hz, 4H), 0.98 (d, J=6.4 Hz, 3H), 0.94-0.85 (m, 6H), 0.85-0.72 (m, 6H), 0.43 (s, 3H). LCMS (ESI): m/z: [M−H] calc'd for C₄₆H₆₂N₈O₇S 870.4; found 871.4.

The following table of compounds (Table 3) were prepared using the aforementioned methods or variations thereof, as is known to those of skill in the art.

TABLE 3
Exemplary Compounds Prepared by
Methods of the Present Invention

| | LCMS (ESI): | | LCMS (ESI): |
|---|---|---|---|
| | m/z [M + H] | | m/z [M + H] |
| Ex# | Found | Ex# | Found |
| A1 | 907.5 | A38 | 835.0 |
| A2 | 963.5 | A39 | 839.7 |
| A3 | 908.3 | A40 | 793.7 |
| A4 | 850.4 | A41 | 878.4 |
| A5 | 892.6 | A42 | 907.4 |
| A6 | 963.5 | A43 | 797.6 |
| A7 | 895.8 | A44 | 807.7 |
| A8 | 949.6 | A45 | 920.5 |
| A9 | 920.5 | A46 | 865.5 |
| A10 | 836.6 | A47 | 894.4 |
| A11 | 894.7 | A48 | 895.8 |
| A12 | 893.5 | A49 | 837.4 |
| A13 | 842.5 | A50 | 853.5 |
| A14 | 949.7 | A51 | 892.5 |
| A15 | 921.5 | A52 | 806.3 |
| A16 | 878.7 | A53 | 798.0 |
| A17 | 864.5 | A54 | 786.5 |
| A18 | 837.6 | A55 | 781.6 |
| A19 | 821.6 | A56 | 821.0 |
| A20 | 894.5 | A57 | 817.5 |
| A21 | 795.4 | A58 | 767.4 |
| A22 | 878.5 | A59 | 823.5 |
| A23 | 880.3 | A60 | 876.6 |
| A24 | 914.6 | A61 | 779.6 |
| A25 | 795.4 | A62 | 863.7 |
| A26 | 837.5 | A63 | 848.6 |
| A27 | 850.5 | A64 | 833.7 |
| A28 | 823.6 | A65 | 866.7 |
| A29 | 906.5 | A66 | 838.4 |
| A30 | 852.6 | A67 | 810.5 |
| A31 | 894.8 | A68 | 838.7 |
| A32 | 836.5 | A69 | 851.7 |
| A33 | | A70 | 823.5 |
| A34 | 906.0 | A71 | 786.5 |
| A35 | 970.7 | A72 | 842.5 |
| A36 | 964.5 | A73 | 864.5 |
| A37 | 971.2 | A74 | 852.5 |
| A75 | 797.6 | A170 | 870.5 |
| A76 | 796.4 | A171 | 879.5 |
| A77 | 822 | A172 | 811.5 |
| A78 | 848.5 | A173 | 871.2 |
| A79 | 904.8 | A174 | 837.4 |
| A80 | 946.5 | A175 | 874.5 |
| A81 | 838.5 | A176 | 807.5 |
| A82 | 853.5 | A177 | 773 |
| A83 | 850.45 | A178 | 787 |
| A84 | 864.5 | A179 | 787 |
| A85 | 864.5 | A180 | 784 |
| A86 | 822.6 | A181 | 784 |
| A87 | 822.3 | A182 | 722.9 |
| A88 | 836.3 | A183 | 722 |
| A89 | 839.6 | A184 | 762 |
| A90 | 837.6 | A185 | 872.18 |
| A91 | 837.5 | A186 | 745.7 |
| A92 | 811.5 | A187 | 829.9 |
| A93 | 811.5 | A188 | 829.9 |
| A94 | 837.5 | A189 | 759.6 |
| A95 | 935.6 | A190 | 775.9 |
| A96 | 919.6 | A191 | 808.7 |
| A97 | 926.5 | A192 | 770.8 |
| A98 | 905.5 | A193 | 802.7 |
| A99 | 912.3 | A194 | 789.8 |
| A100 | 864.5 | A195 | 796.7 |
| A101 | 852.5 | A196 | 744.7 |
| A102 | 795.4 | A197 | 798.9 |
| A103 | 772.3 | A198 | 840.9 |
| A104 | 781.4 | A199 | 753.9 |
| A105 | 891.5 | A200 | 758.9 |
| A106 | 898.5 | A201 | 984.4 |
| A107 | 848.5 | A202 | 934.4 |
| A108 | 855.5 | A203 | 941.5 |
| A109 | 878.8 | A204 | 950.4 |
| A110 | 885.6 | A205 | 857.3 |
| A111 | 894.6 | A206 | 890.4 |
| A112 | 947.7 | A207 | 791.7 |
| A113 | 954.7 | A208 | 793.6 |
| A114 | 963.6 | A209 | 867.5 |
| A115 | 892.4 | A210 | 858.5 |
| A116 | 889.5 | A211 | 922.6 |
| A117 | 936.5 | A212 | 798.4 |
| A118 | 841.4 | A213 | 867.7 |
| A119 | 834.8 | A214 | 797.5 |
| A120 | 921.5 | A215 | 946.5 |
| A121 | 852.8 | A216 | 904.8 |
| A122 | 865.8 | A217 | 862.6 |
| A123 | 907.8 | A218 | 835.5 |
| A124 | 851.8 | A219 | 849.6 |
| A125 | 838 | A220 | 931.4 |
| A126 | 862.5 | A221 | 911.3 |
| A127 | 864.8 | A222 | 853.2 |
| A128 | 864.8 | A223 | 835.5 |
| A129 | 850.8 | A224 | 821.6 |
| A130 | 906 | A225 | 748.8 |
| A131 | 865.8 | A226 | 913.8 |
| A132 | 838.9 | A227 | 894.0 |
| A133 | 877.9 | A228 | 877.9 |
| A134 | 879.8 | A229 | 897.8 |
| A135 | 961.6 | A230 | 879.9 |
| A136 | 815.5 | A231 | 893.9 |
| A137 | 801.5 | A232 | 852.9 |
| A138 | 802.4 | A233 | 950.6 |
| A139 | 850.5 | A234 | 917.3 |
| A140 | 862.6 | A235 | 897.3 |
| A141 | 811.4 | A236 | 780.8 |
| A142 | 793.3 | A237 | 919.4 |
| A143 | 856.2 | A238 | 842.4 |
| A144 | 793.5 | A239 | 826.4 |
| A145 | 836.2 | A240 | 851.8 |
| A146 | 835.4 | A241 | 851.7 |
| A147 | 835.3 | A242 | 878.9 |
| A148 | 876.6 | A243 | 864.8 |
| A149 | 862.6 | A244 | 883.5 |
| A150 | 865.5 | A245 | 828.4 |
| A151 | 890.3 | A246 | 821.4 |
| A152 | 786.2 | A247 | 912.8 |
| A153 | 819.5 | A248 | 893.6 |
| A154 | 857.2 | A249 | 888.8 |
| A155 | 862.6 | A250 | 899.8 |
| A156 | 847.5 | A251 | 864.7 |
| A157 | 849.5 | A252 | 905.8 |
| A158 | 849.5 | A253 | 750.7 |
| A159 | 846.6 | A254 | 787.8 |
| A160 | 839.6 | A255 | 851.6 |
| A161 | 839.5 | A256 | 795.4 |
| A162 | 839.5 | A257 | 852.6 |
| A163 | 862.6 | A258 | 766.8 |
| A164 | 862.7 | A259 | 864.5 |
| A165 | 839.5 | A260 | 853.4 |
| A166 | 857.5 | A261 | 773.8 |
| A167 | 857.5 | A262 | 878.7 |
| A168 | 836.5 | A263 | 780.8 |
| A169 | 880.3 | | |
| A264 | 758.4 | A293 | 898.7 |
| A265 | 757.3 | A294 | 912.7 |
| A266 | 772.4 | A295 | 882.3 |
| A267 | 728.4 | A296 | 912.3 |
| A268 | 882.4 | A297 | 921.3 |
| A270 | 744.3 | A298 | 883.2 |
| A271 | 871.2 | A299 | 871.3 |
| A272 | 898.6 | A300 | 898.5 |
| A273 | 910.5 | A301 | 869.3 |
| A274 | 882.3 | A302 | 893.5 |
| A275 | 885.5 | A303 | 924.4 |
| A276 | 885.5 | A304 | 841.2 |
| A277 | 913.6 | A305 | 841.5 |
| A278 | 885.6 | A306 | 914.5 |
| A279 | 885.4 | A307 | 896.5 |
| A280 | 910.6 | A308 | 896.4 |
| A281 | 884.3 | A309 | 871.3 |
| A282 | 882.2 | A310 | 871.4 |
| A283 | 898.5 | A311 | 896.5 |
| A284 | 882.5 | A312 | 883.5 |
| A285 | 896.2 | A313 | 896.6 |
| A286 | 713.1 | A314 | 882.6 |
| A287 | 835.3 | A315 | 729.3 |
| A288 | 925.4 | A316 | 906.5 |
| A289 | 885.0 | A317 | 827.4 |
| A290 | 941.3 | A318 | 898.5 |
| A291 | 898.3 | A319 | 898.3 |
| A292 | 898.7 | A320 | 733.2 |
| A321 | 771.4 | A354 | 844.6 |
| A322 | 893.2 | A355 | 850.5 |
| A323 | 837.5 | A356 | 855.5 |
| A324 | 807.3 | A357 | 905.4 |
| A325 | 922.5 | A358 | 843.5 |
| A326 | 882.5 | A359 | 715.2 |
| A327 | 882.5 | A360 | 715.2 |
| A328 | 924.4 | A361 | 731.3 |
| A329 | 896.3 | A362 | 717.3 |
| A330 | 911.1 | A363 | 855.5 |
| A331 | 729.4 | A364 | 866.5 |
| A332 | 857.4 | A365 | 908.6 |
| A333 | 857.5 | A366 | 736.1 |
| A334 | 857.2 | A367 | 699.1 |
| A335 | 871.5 | A368 | 714.1 |
| A336 | 829.5 | A369 | 713.3 |
| A337 | 856.5 | A370 | 947.6 |
| A338 | 912.2 | A371 | 961.4 |
| A339 | 857.5 | A372 | 857.5 |
| A340 | 771.4 | A373 | 857.5 |
| A341 | 870.5 | A374 | 857.5 |
| A342 | 975.3 | A375 | 856.5 |
| A343 | 842.5 | A376 | 857.5 |
| A344 | 871.7 | A377 | 865.6 |
| A345 | 808.5 | A378 | 947.3 |
| A346 | 837.5 | A379 | 975.6 |
| A347 | 837.5 | A380 | 961.3 |
| A348 | 963.5 | A381 | 850.6 |
| A349 | 855.5 | A382 | 852.2 |
| A350 | 843.5 | A383 | 905.1 |
| A351 | 843.5 | A384 | 849.5 |
| A352 | 855.5 | A385 | 961.6 |
| A353 | 841.5 | A386 | 949.3 |
| A387 | 871.5 | A421 | 892.9 |
| A388 | 819.3 | A422 | 951.3 |
| A389 | 813.2 | A423 | 1051.6 |
| A391 | 851.7 | A424 | 939.4 |
| A392 | 851.7 | A425 | 927.4 |
| A393 | 891.8 | A426 | 953.40 |
| A394 | 879.8 | A427 | 978.3 |
| A395 | 879.8 | A428 | 918.2 |
| A396 | 921.8 | A429 | 911.3 |
| A397 | 909.8 | A430 | 804.5 |
| A398 | 736.4 | A431 | 891.5 |
| A399 | 827.5 | A432 | 879.5 |
| A400 | 841.5 | A433 | 940.7 |
| A401 | 857.5 | A434 | 896.5 |
| A402 | 871.4 | A435 | 896.3 |
| A403 | 871.5 | A436 | 926.2 |
| A404 | 906.9 | A437 | 946.6 |
| A405 | 865.9 | A438 | 896.1 |
| A406 | 863.8 | A439 | 988.1 |
| A407 | 891.9 | A440 | 988.1 |
| A408 | 919.9 | A441 | 926.2 |
| A409 | 908 | A442 | 910.9 |
| A410 | 878.0 | A443 | 967.1 |
| A411 | 878.0 | A444 | 912.1 |
| A412 | 878.0 | A445 | 912.1 |
| A413 | 922.0 | A446 | 882.2 |
| A414 | 894.9 | A447 | 867.1 |
| A415 | 928.0 | A448 | 953.2 |
| A416 | 901.9 | A449 | 953.5 |
| A417 | 890.9 | A450 | 1017.2 |
| A418 | 867.9 | A451 | 912.2 |
| A419 | 879.9 | A452 | 895.2 |
| A420 | 866.9 | A453 | 924.2 |
| A454 | 844.2 | A482 | 960.1 |
| A455 | 901.9 | A483 | 1008.1 |
| A456 | 867.2 | A484 | 912.2 |
| A457 | 940.6 | A485 | 938.6 |
| A458 | 898.5 | A486 | 952.3 |
| A459 | 954.8 | A487 | 885.3 |
| A460 | 896.2 | A488 | 884.3 |
| A461 | 924.2 | A489 | 886.2 |
| A462 | 896.2 | A490 | 1017.9 |
| A463 | 856.2 | A491 | 912.6 |
| A464 | 931.2 | A492 | 912.6 |
| A465 | 981.7 | A493 | 912.6 |
| A466 | 955.3 | A494 | 912.6 |
| A467 | 940.6 | A495 | 924.2 |
| A468 | 910.6 | A496 | 917.0 |
| A469 | 884.5 | A497 | 882.1 |
| A470 | 896.2 | A498 | 924.6 |
| A471 | 896.1 | A499 | 912.1 |
| A472 | 912.1 | A500 | 921.2 |
| A473 | 898.6 | A501 | 984.1 |
| A474 | 899.2 | A502 | 884.6 |
| A475 | 899.1 | A503 | 896.1 |
| A476 | 996.3 | A504 | 898.1 |
| A477 | 968.6 | A505 | 954.7 |
| A478 | 885.5 | A506 | 902.1 |
| A479 | 910.9 | A507 | 1011.2 |
| A480 | 910.1 | A508 | 884.6 |
| A481 | 896.9 | A509 | 943.2 |
| A510 | 883.5 | A538 | 898.2 |
| A511 | 952.3 | A539 | 896.5 |
| A512 | 940.6 | A540 | 870.0 |
| A513 | 910.3 | A541 | 882.1 |
| A514 | 901.2 | A542 | 884.2 |
| A515 | 901.2 | A543 | 940.9 |
| A516 | 896.3 | A544 | 874.2 |
| A517 | 896.2 | A545 | 897.9 |
| A518 | 898.6 | A546 | 928.2 |
| A519 | 898.6 | A547 | 912.5 |
| A520 | 911.2 | A548 | 912.5 |
| A521 | 897.2 | A549 | 920.5 |
| A522 | 883.2 | A550 | 934.5 |
| A523 | 853.2 | A551 | 934.4 |
| A524 | 946.2 | A552 | 920.5 |
| A525 | 896.2 | A553 | 887.1 |
| A526 | 940.3 | A554 | 837.4 |
| A527 | 871.2 | A555 | 955.2 |
| A528 | 883.2 | A556 | 932.2 |
| A529 | 951.8 | A557 | 906.1 |
| A530 | 945.1 | A558 | 856.2 |
| A531 | 898.3 | A559 | 888.2 |
| A532 | 954.5 | A560 | 869.1 |
| A533 | 899.5 | A561 | 883.2 |
| A534 | 899.5 | A562 | 1009.3 |
| A535 | 884.2 | A563 | 884.6 |
| A536 | 939.5 | A564 | 884.6 |
| A537 | 939.5 | A565 | 924.2 |
| A566 | 884.9 | A592 | 961.2 |
| A567 | 874.2 | A593 | 1043.0 |
| A568 | 896.2 | A594 | 1025.8 |
| A569 | 898.2 | A595 | 967.5 |
| A570 | 870.2 | A596 | 967.5 |
| A571 | 899.0 | A597 | 900.1 |
| A572 | 914.2 | A598 | 845.4 |
| A573 | 912.2 | A599 | 917.2 |
| A574 | 913.9 | A600 | 933.1 |
| A575 | 914.2 | A601 | 898.0 |
| A576 | 885.0 | A602 | 897.0 |
| A577 | 1024.7 | A603 | 884.9 |
| A578 | 869.5 | A604 | 933.1 |
| A579 | 871.4 | A605 | 926.1 |
| A580 | 886.9 | A606 | 940.5 |
| A581 | 872.1 | A607 | 924.3 |
| A582 | 933.2 | A608 | 896.3 |
| A583 | 1016.2 | A609 | 898.4 |
| A584 | 927.2 | A610 | 898.5 |
| A585 | 918.2 | AS11 | 870.5 |
| A586 | 911.3 | A612 | 858.4 |
| A587 | 899.4 | A613 | 858.3 |
| A588 | 898.6 | A614 | 899.4 |
| A589 | 910.2 | A615 | 926.4 |
| A590 | 914.6 | A616 | 926.4 |
| A591 | 915.4 | | |

Blank = not determined

### Biological Assays

Potency Assay: pERK

The purpose of this assay is to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-1H358 cells is applicable to K-Ras G12C.

Note: this protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO2 incubator. Test compounds were prepared in 10, 3-fold dilutions in DMSO, with a high concentration of 10 mM. On day of assay, 40 nl of test compound was added to each well of cell culture plate using an Echo550 liquid handler (LabCyte®). Concentrations of test compound were tested in duplicate. After compound addition, cells were incubated 4 hours at 37° C., 5% CO2. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μl lysis buffer, with shaking at 600 RPM at room temperature. Lysate (10 μl) was transferred to a 384-well Opti-plate (PerkinElmer) and 5 μl acceptor mix was added. After a 2-hour incubation in the dark, 5 μl donor mix was added, plate was sealed and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μl tris buffered saline (TBS) and fixed for 15 minutes with 150 μl 4% paraformaldehyde in TBS. Fixed cells were washed 4 times for 5 minutes with TBS containing 0.1% Triton X-100 (TBST) and then blocked with 100 μl Odyssey blocking buffer (LI-COR) for 60 minutes at room temperature. Primary antibody (pERK, CST-4370, Cell Signaling Technology) was diluted 1:200 in blocking buffer, and 50 μl was added to each well and incubated overnight at 4° C. Cells were washed 4 times for 5 minutes with TBST. Secondary antibody (IR-8000W rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a LI-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), Capan-1 (K-Ras G12V)) over a 5-day treatment period by quantifying the amount of ATP present at endpoint using the CellTiter-Glo®2.0 Reagent (Promega).

Cells were seeded at 250 cells/well in 40 μl of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μl) were transferred to the next wells containing 30 μl of 100% DMSO, and repeated until a 9-concentration 3-fold serial dilution was made (starting assay concentration of 10 μM). Test compounds (132.5 nl) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μl) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD}D construct, inhibiting K-Ras signaling through a RAF effector. Data is reported as IC50 values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} were combined in a 384-well assay plate at final concentrations of 25 μM, 12.5 nM and 50 nM, respectively. Compound was present in plate wells as a 10-point 3-fold dilution series starting at a final concentration of 30 μM. After incubation at 25° C. for 3 hours, a mixture of Anti-His Eu-W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras:RAF complex were identified as those eliciting a decrease in the TR-FRET ratio relative to DMSO control wells.

TABLE 4
Biological Assay Date for Representative Compounds of the Present Invention

| | | | | | | | | | H358 Cell |
|---|---|---|---|---|---|---|---|---|---|
| | FRET G12C | FRET G12V | FRET G12D | FRET G13C | FRET G13D | H358 pERK | Capan-1 pERK | ASPC-1 pERK | Viability |
| Ex# | IC50, uM | IC50, uM | IC50, uM | IC50, uM | IC50, uM | EC50, uM | EC50, uM | EC50, uM | IC50, uM |
| A74 | 0.45 | 3.67 | 0.228 | 0.091 | 0.212 | 0.334 | 0.413 | 0.468 | |
| A73 | 0.082 | 0.905 | 0.061 | 0.013 | 0.033 | 0.036 | 0.197 | 0.079 | |
| A3 | 0.029 | 0.043 | 0.545 | 0.099 | 0.16 | 0.018 | 0.008 | 0.144 | 0.057 |
| A25 | 0.128 | 0.197 | 1.29 | 0.097 | 0.235 | 0.064 | 0.052 | 0.706 | 0.226 |
| A12 | 0.068 | 0.329 | 0.154 | 0.148 | 0.219 | | | | 4.29 |

Blank = not determined

### Additional H358 Cell Viability Assay Data

### *Key:

+++++: IC50≥10 uM+

++++: 10 uM>IC50≥1 uM+

+++: 1 uM>IC50≥0.1 uM+

++: 0.1 uM>IC50≥0.01 uM+

+: IC50<0.01 uM

TABLE 5
H358 Cell Viability assay data (K-Ras G12C, IC50, uM):

| IC50* | Examples |
|---|---|
| + | A136, A159, A205, A277, A278, A289, A291, A296, A298, A302, A303, |
| | A304, A306, A309, A310, A325, A335, A338, A356, A358, A365, A372, |
| | A373, A374, A382, A399, A439, A443, A450, A457, A465, A466, A476, |
| | A477, A478, A483, A484, A487, A490, A500, A501, A505, A514, A515, |
| | A526, A529, A536, A543, A546, A551, A555, A561, A562, A573, A577, |
| | A583, A590, A593, A594, A606, A607 |
| ++ | A114, A117, A13, A131, A138, A141, A147, A156, A160, A162, A164, |
| | A165, A170, A202, A204, A211, A217, A218, A223, A224, A233, A240, |
| | A242, A247, A248, A249, A250, A252, A273, A279, A280, A285, A286, |
| | A288, A290, A293, A294, A295, A297, A299, A3, A301, A305, A307, |
| | A311, A312, A313, A316, A318, A319, A326, A329, A330, A333, A334, |
| | A336, A337, A342, A348, A349, A350, A351, A352, A353, A357, A363, |
| | A364, A375, A376, A377, A379, A383, A387, A389, A391, A392, A400, |
| | A401, A402, A403, A406, A415, A422, A433, A436, A440, A441, A444, |
| | A445, A451, A452, A454, A458, A459, A467, A481, A485, A486, A488, |
| | A489, A491, A492, A493, A494, A495, A498, A499, A502, A503, A506, |
| | A507, A509, A510, A511, A512, A513, A518, A520, A521, A522, A523, |
| | A525, A527, A528, A531, A532, A533, A534, A537, A538, A540, A541, |
| | A542, A547, A548, A549, A550, A552, A553, A557, A559, A560, A563, |
| | A564, A565, A566, A567, A568, A569, A570, A571, A574, A576, A578, |
| | A582, A584, A585, A587, A588, A589, A591, A595, A596, A597, A601, |
| | A603, A605, A614, A615 |
| +++ | A100, A11, A116, A121, A123, A124, A126, A130, A132, A137, A139, |
| | A143, A146, A152, A155, A157, A161, A166, A167, A168, A169, A171, |
| | A173, A174, A18, A184, A19, A201, A203, A209, A21, A210, A219, |
| | A221, A226, A228, A231, A232, A238, A239, A241, A243, A245, A25, |
| | A252, A26, A260, A264, A266, A267, A268, A270, A274, A276, A28, |
| | A281, A282, A283, A284, A287, A29, A292, A30, A308, A314, A315, |
| | A317, A321, A322, A323, A332, A339, A343, A346, A35, A354, A355, |
| | A360, A361, A362, A367, A368, A369, A378, A381, A384, A385, A386, |
| | A39, A393, A395, A396, A397, A407, A408, A409, A410, A411, A412, |
| | A413, A414, A416, A417, A418, A420, A421, A423, A437, A438, A442, |
| | A447, A449, A455, A461, A462, A471, A480, A482, A496, A497, A5, |
| | A504, A508, A524, A535, A539, A54, A544, A545, A554, A556, A572, |
| | A575, A579, A580, A581, A586, A592, A598, A600, A602, A604, A608, |
| | A610, A611, A612, A613, A616, A64, A7, A78, A8, A90, A91, A94, A95 |
| ++++ | A1, A10, A101, A102, A104, A111, A117, A12, A120, A125, A127, A128, |
| | A129, A134, A148, A15, A16, A163, A17, A2, A20, A216, A22, A227, A23, |
| | A24, A244, A254, A254, A256, A258, A259, A261, A262, A27, A3, A320, |
| | A359, A36, A36, A37, A38, A4, A40, A405, A41, A42, A43, A44, A45, A453, |
| | A46, A464, A50, A51, A517, A519, A52, A53, A54, A55, A57, A58, A59, |
| | A599, A6, A60, A609, A61, A65, A66, A67, A70, A82, A83, A85, A9, A92, A97 |
| +++++ | A12, A133, A14, A31, A32, A4, A47, A48, A56, A62, A63, A68, A69, A84, A99 |

*Key:
+++++: IC50 ≥ 10 uM
++++: 10 uM > IC50 ≥ 1 uM
+++: 1 uM > IC50 ≥ 0.1 uM
++: 0.1 uM > IC50 ≥ 0.01 uM
+: IC50 < 0.01 uM

TABLE 6
Capan-1 Cell Viability assay data (K-Ras G12V, IC50, uM):

| IC50* | Examples |
|---|---|
| + | A277, A450, A465, A466, A476, A477, A484, A500, A505, A526, A529, |
| | A555, A562, A577, A583, A593, A594 |
| ++ | A114, A117, A132, A136, A138, A141, A156, A159, A162, A165, A170, |
| | A202, A204, A205, A210, A211, A218, A224, A233, A240, A247, A250, |
| | A278, A279, A280, A285, A288, A289, A290, A291, A293, A295, A296, |
| | A298, A3, A302, A303, A304, A306, A309, A310, A312, A313, A316, A318, |
| | A319, A325, A329, A330, A334, A335, A336, A338, A353, A356, A357, |
| | A358, A363, A364, A365, A372, A373, A374, A376, A377, A382, A383, |
| | A387, A389, A399, A400, A401, A402, A403, A415, A433, A436, A439, |
| | A440, A443, A444, A445, A451, A452, A454, A457, A458, A467, A472, |
| | A474, A475, A478, A481, A483, A485, A486, A487, A490, A491, A494, |
| | A499, A503, A506, A509, A510, A511, A512, A513, A514, A515, A518, |
| | A520, A521, A523, A525, A527, A528, A531, A532, A533, A534, A536, |
| | A537, A538, A540, A543, A546, A547, A548, A549, A550, A551, A561, |
| | A563, A565, A566, A569, A570, A571, A573, A574, A576, A587, A590, |
| | A591, A601, A603, A606, A607, A608, A614, A615 |
| +++ | A102, A11, A116, A121, A123, A126, A13, A131, A137, A139, A143, A146, |
| | A147, A152, A157, A160, A161, A164, A166, A167, A168, A169, A171, A173, |
| | A174, A18, A19, A201, A203, A209, A21, A217, A219, A221, A223, A226, |
| | A228, A232, A238, A239, A241, A242, A244, A245, A248, A249, A25, A252, |
| | A252, A254, A26, A264, A266, A267, A268, A273, A274, A275, A276, A281, |
| | A282, A283, A284, A286, A287, A292, A294, A297, A299, A30, A301, A305, |
| | A307, A308, A311, A314, A315, A320, A321, A322, A323, A326, A332, A333, |
| | A337, A342, A343, A346, A347, A348, A349, A350, A351, A352, A354, A355, |
| | A360, A361, A362, A367, A368, A369, A375, A379, A384, A385, A386, A39, |
| | A395, A396, A397, A406, A407, A409, A410, A411, A412, A413, A416, A419, |
| | A420, A422, A423, A437, A441, A447, A449, A455, A459, A461, A462, A468, |
| | A469, A470, A471, A473, A480, A482, A488, A489, A492, A493, A495, A496, |
| | A497, A498, A5, A501, A502, A504, A507, A508, A516, A517, A522, A524, |
| | A530, A535, A539, A54, A541, A542, A544, A545, A552, A553, A556, A557, |
| | A558, A559, A560, A564, A567, A568, A575, A578, A579, A580, A582, A584, |
| | A585, A586, A588, A589, A595, A596, A597, A598, A600, A602, A605, A609, |
| | A610, A611, A612, A613, A616, A90, A91, A94, A95 |
| ++++ | A1, A100, A101, A106, A107, A111, A112, A113, A115, A124, A125, A129, A130, |
| | A135, A144, A148, A149, A155, A158, A16, A17, A175, A176, A179, A180, A182, |
| | A183, A184, A185, A200, A216, A220, A225, A225, A229, A230, A231, A235, A236, |
| | A237, A24, A246, A253, A254, A259, A260, A261, A262, A265, A270, A272, A28, |
| | A29, A300, A317, A324, A327, A331, A339, A345, A359, A366, A370, A371, A378, |
| | A380, A381, A388, A391, A392, A393, A394, A398, A40, A405, A408, A414, A417, |
| | A418, A421, A43, A434, A435, A438, A442, A453, A456, A460, A463, A464, A479, |
| | A519, A54, A554, A572, A581, A592, A599, A604, A61, A7, A76, A78, A8, A80, |
| | A82, A83, A85, A89, A93, A96, A97 |
| +++++ | A104, A104, A105, A108, A109, A110, A116, A117, A118, A119, A120, A122, |
| | A127, A128, A133, A134, A140, A142, A145, A150, A151, A153, A154, A163, |
| | A172, A177, A178, A178, A179, A181, A186, A187, A188, A189, A190, A191, |
| | A192, A193, A194, A195, A196, A197, A198, A199, A206, A207, A208, A212, |
| | A213, A214, A215, A222, A227, A227, A234, A236, A243, A251, A253, A255, |
| | A258, A271, A328, A340, A341, A344, A4, A404, A446, A448, A75, A79, A84, |
| | A86, A87, A87, A88, A92, A98, A99 |

### Additional Ras-Raf Disruption/FRET/MOA Assay Data (IC50, uM):

### *Key:

+++++: IC50≥10 uM+

++++: 10 uM>IC50≥1 uM+

+++: 1 uM>IC50≥0.1 uM+

++: 0.1 uM>IC50≥0.01 uM+

+: IC50<0.01 uM

TABLE 7
KRAS G12D FRET data

| IC50* | Examples |
|---|---|
| + | None |
| ++ | A1, A100, A111, A120, A124, A125, A127, A128, A129, A131, A133, A134, |
| | A135, A139, A140, A148, A159, A164, A223, A227, A228, A231, A242, A243, |
| | A247, A249, A325, A342, A348, A365, A370, A371, A378, A379, A380, A381, |
| | A385, A386, A391, A392, A393, A395, A397, A4, A415, A419, A427, A483, |
| | A494, A501, A507, A546, A573, A577, A584, A594, A605, A95 |
| +++ | A10, A101, A102, A106, A114, A12, A121, A122, A123, A126, A130, A132, |
| | A136, A14, A146, A147, A149, A15, A151, A155, A156, A157, A158, A160, |
| | A161, A162, A163, A165, A166, A167, A168, A169, A171, A174, A2, A201, |
| | A202, A204, A205, A209, A211, A216, A217, A218, A219, A224, A227, A229, |
| | A23, A230, A232, A233, A240, A241, A248, A248, A250, A251, A252, A252, |
| | A255, A259, A264, A265, A266, A267, A268, A27, A270, A273, A274, A275, |
| | A277, A278, A279, A280, A285, A286, A287, A288, A289, A290, A291, A294, |
| | A298, A3, A302, A303, A304, A306, A309, A31, A310, A311, A312, A313, A314, |
| | A32, A321, A323, A332, A333, A334, A335, A336, A343, A346, A347, A349, |
| | A350, A351, A353, A356, A358, A363, A364, A372, A373, A374, A376, A377, |
| | A382, A383, A384, A394, A396, A399, A400, A401, A402, A404, A405, A406, |
| | A407, A408, A409, A41 , A410, A411, A412, A413, A414, A416, A417, A418, |
| | A420, A421, A422, A423, A424, A426, A432, A434, A435, A436, A438, A441, |
| | A443, A444, A447, A45, A450, A454, A457, A458, A459, A463, A465, A466, |
| | A467, A468, A469, A471, A475, A476, A477, A478, A48, A484, A485, A487, |
| | A488, A491, A492, A493, A498, A5, A500, A502, A503, A505, A506, A509, |
| | A514, A515, A518, A520, A523, A526, A528, A529, A531, A533, A534, A536, |
| | A537, A538, A542, A543, A545, A549, A551, A552, A554, A555, A557, A558, |
| | A559, A560, A561, A562, A563, A564, A565, A566, A567, A568, A569, A571, |
| | A574, A576, A578, A580, A581, A582, A583, A586, A587, A588, A589, A590, |
| | A591, A593, A595, A596, A6, A600, A601, A603, A606, A607, A608, A610, |
| | A611, A614, A615, A616, A62, A66, A67, A68, A7, A78, A79, A8, A80, A81, |
| | A83, A85, A87, A87, A88, A89, A99 |
| ++++ | A105, A107, A108, A109, A11, A110, A112, A113, A117, A118, A119, A12, |
| | A13, A137, A138, A144, A152, A16, A17, A170, A173, A175, A176, A177, |
| | A178, A179, A179, A18, A184, A19, A20, A208, A21, A210, A213, A215, A22, |
| | A222, A225, A226, A236, A239, A24, A25, A253, A254, A254, A257, A26, |
| | A260, A262, A272, A276, A28, A282, A283, A284, A292, A293, A295, A296, |
| | A297, A299, A30, A300, A301, A305, A307, A308, A315, A316, A318, A319, |
| | A320, A322, A324, A326, A329, A33, A330, A331, A337, A338, A339, A344, |
| | A345, A35, A352, A354, A355, A357, A359, A36, A36, A360, A361, A362, |
| | A366, A367, A368, A369, A375, A38, A387, A389, A39, A4, A40, A403, A425, |
| | A428, A43, A431, A433, A437, A439, A44, A440, A442, A445, A448, A449, |
| | A451, A452, A455, A456, A46, A460, A461, A462, A464, A47, A470, A472, |
| | A473, A474, A479, A480, A481, A486, A489, A49, A490, A495, A496, A497, |
| | A499, A50, A504, A508, A51, A510, A511, A512, A513, A516, A517, A519, |
| | A521, A522, A524, A525, A527, A530, A532, A535, A539, A540, A541, A544, |
| | A547, A548, A550, A553, A556, A570, A572, A575, A579, A585, A592, A597, |
| | A598, A599, A602, A604, A609, A612, A64, A65, A69, A70, A76, A82, A84, |
| | A9, A90, A91, A92, A93, A94, A96, A97, A98, A613 |
| +++++ | A103, A103, A104, A104, A115, A116, A116, A117, A141, A142, A143, A145, |
| | A150, A153, A154, A172, A178, A180, A181, A182, A183, A185, A186, A187, |
| | A188, A189, A190, A191, A192, A193, A194, A195, A196, A197, A198, A199, |
| | A200, A203, A206, A207, A212, A214, A220, A221, A225, A234, A235, A236, |
| | A237, A238, A244, A245, A246, A253, A256, A258, A261, A271, A281, A29, |
| | A3, A317, A327, A328, A340, A341, A37, A388, A398, A42, A429, A430, A446, |
| | A453, A482, A52, A53, A54, A54, A55, A56, A57, A58, A59, A60, A61, A63, |
| | A75, A77, A86 |

TABLE 8
KRAS G12C FRET data

| IC50* | Examples |
|---|---|
| + | A323, A325, A347, A501, A546, A577, A594 |
| ++ | A1, A10, A100, A11, A111, A114, A117, A12, A120, A121, A125, |
| | A126, A127, A128, A129, A13, A131, A132, A135, A136, A139, A14, |
| | A140, A146, A147, A148, A149, A15, A151, A155, A156, A157, A159, |
| | A16, A160, A162, A164, A165, A166, A168, A17, A18, A19, A2, A20, |
| | A201, A202, A204, A205, A211, A216, A217, A218, A219, A223, A224, |
| | A226, A227, A228, A229, A230, A231, A233, A240, A241, A242, A243, |
| | A247, A248, A248, A249, A250, A252, A252, A255, A262, A264, A265, |
| | A266, A273, A274, A275, A277, A278, A279, A280, A285, A288, A289, |
| | A290, A291, A298, A3, A302, A303, A304, A306, A309, A310, A312, |
| | A316, A321, A330, A333, A334, A335, A336, A338, A342, A343, A346, |
| | A348, A349, A350, A351, A353, A356, A358, A363, A364, A365, A370, |
| | A371, A372, A373, A374, A376, A377, A378, A379, A380, A381, A382, |
| | A383, A384, A385, A386, A387, A391, A392, A393, A395, A396, A397, |
| | A399, A4, A400, A401, A402, A405, A406, A407, A408, A409, A410, |
| | A411, A412, A413, A414, A415, A418, A419, A420, A422, A424, A426, |
| | A427, A432, A438, A443, A444, A450, A452, A454, A457, A458, A459, |
| | A465, A466, A467, A471, A475, A477, A478, A483, A484, A487, A488, |
| | A489, A491, A493, A494, A498, A5, A500, A503, A505, A507, A509, |
| | A510, A514, A515, A523, A526, A528, A529, A533, A534, A536, A537, |
| | A538, A540, A543, A549, A550, A551, A552, A554, A555, A557, A558, |
| | A560, A561, A562, A565, A567, A569, A571, A573, A574, A576, A578, |
| | A581, A582, A583, A584, A586, A590, A591, A593, A595, A596, A598, |
| | A6, A600, A601, A605, A606, A607, A608, A614, A615, A616, A62, A67, |
| | A68, A7, A8, A87, A9, A95 |
| +++ | A101, A102, A105, A106, A12, A122, A123, A124, A130, A133, A134, A137, |
| | A138, A144, A158, A161, A163, A167, A169, A170, A171, A173, A174, A176, |
| | A178, A179, A179, A182, A183, A184, A207, A208, A209, A21, A210, A215, |
| | A22, A225, A227, A23, A232, A236, A24, A25, A251, A253, A254, A254, A257, |
| | A259, A26, A260, A267, A268, A27, A270, A276, A28, A282, A283, A284, A286, |
| | A287, A29, A292, A293, A294, A295, A296, A297, A299, A30, A301, A305, A308, |
| | A31, A311, A313, A314, A315, A318, A319, A32, A320, A322, A324, A326, A329, |
| | A33, A331, A332, A337, A339, A34, A344, A345, A35, A352, A354, A355, A357, |
| | A359, A36, A36, A360, A361, A362, A366, A367, A368, A369, A37, A375, A38, |
| | A389, A39, A394, A4, A40, A403, A404, A41, A416, A417, A42, A421, A423, A425, |
| | A43, A431, A433, A434, A435, A436, A437, A439, A44, A440, A441, A445, A447, |
| | A449, A45, A451, A453, A455, A456, A46, A460, A461, A462, A463, A468, A469, |
| | A47, A472, A473, A474, A476, A479, A48, A480, A481, A485, A486, A49, A490, |
| | A492, A495, A496, A497, A499, A50, A502, A504, A506, A508, A51, A511, A512, |
| | A513, A518, A519, A52, A520, A521, A522, A524, A525, A527, A53, A530, A531, |
| | A532, A535, A541, A542, A544, A545, A547, A548, A553, A556, A559, A563, A564, |
| | A566, A568, A570, A579, A580, A585, A587, A588, A589, A592, A597, A602, A603, |
| | A610, A611, A612, A64, A65, A66, A69, A76, A78, A79, A80, A81, A82, A83, A84, |
| | A85, A86, A87, A88, A89, A90, A91, A93, A94, A96, A97, A98, A99 |
| ++++ | A104, A107, A108, A109, A110, A112, A113, A115, A116, A117, A118, A119, A141, |
| | A142, A143, A150, A152, A175, A177, A178, A180, A181, A185, A199, A203, A206, |
| | A212, A213, A214, A220, A221, A222, A225, A236, A237, A238, A239, A244, A245, |
| | A246, A253, A256, A258, A261, A271, A272, A281, A3, A300, A307, A317, A388, |
| | A398, A428, A429, A442, A446, A448, A464, A470, A482, A516, A517, A539, A54, |
| | A54, A55, A56, A57, A572, A575, A58, A59, A599, A60, A604, A609, A61, A63, |
| | A70, A75, A77, A92, A613 |
| +++++ | A103, A103, A104, A116, A145, A153, A154, A172, A186, A187, A188, A189, A190, |
| | A191, A192, A193, A194, A195, A196, A197, A198, A200, A234, A235, A327, A328, |
| | A340, A341, A430 |

TABLE 9
KRAS G12S FRET data

| IC50* | Examples |
|---|---|
| + | A501, A577, A594 |
| ++ | A1, A10, A100, A111, A114, A120, A121, A124, A125, A127, A128, A129, A131, A135, A139, A140, |
| | A147, A148, A156, A159, A162, A164, A165, A2, A202, A204, A211, A217, A218, A219, A223, A224, |
| | A227, A228, A230, A242, A243, A247, A248, A248, A249, A250, A252, A252, A273, A275, A277, A291, |
| | A3, A312, A323, A325, A335, A342, A347, A348, A349, A351, A363, A365, A370, A371, A377, A378, |
| | A379, A380, A381, A385, A386, A391, A392, A393, A395, A396, A397, A4, A400, A405, A406, A407, |
| | A408, A409, A411, A414, A415, A418, A419, A422, A424, A427, A459, A465, A483, A491, A494, A498, |
| | A5, A500, A503, A507, A526, A529, A537, A546, A554, A555, A558, A561, A565, A573, A578, A584, |
| | A590, A6, A605, A606, A607, A615, A68, A7, A87, A95 |
| +++ | A101, A102, A11, A117, A119, A12, A122, A123, A126, A13, A130, A132, A133, A134, A136, A14, A146, |
| | A149, A15, A151, A155, A157, A158, A16, A160, A161, A163, A166, A167, A168, A169, A17, A170, A171, |
| | A173, A174, A18, A184, A19, A20, A201, A205, A209, A21, A215, A216, A22, A226, A227, A229, A23, |
| | A231, A232, A233, A236, A24, A240, A241, A25, A251, A254, A254, A255, A257, A259, A26, |
| | A260, A262, A264, A265, A266, A267, A268, A27, A270, A274, A276, A278, A279, A28, A280, A284, |
| | A285, A286, A287, A288, A289, A29, A290, A293, A294, A295, A296, A297, A298, A30, A301, A302, |
| | A303, A304, A306, A309, A31, A310, A311, A313, A314, A315, A316, A318, A319, A32, A320, A321, |
| | A324, A326, A329, A33, A330, A331, A332, A333, A334, A336, A337, A338, A343, A344, A346, A35, |
| | A350, A352, A353, A354, A356, A357, A358, A36, A360, A361, A362, A364, A367, A368, A369, A37, |
| | A372, A373, A374, A375, A376, A38, A382, A383, A384, A387, A39, A394, A399, A40, A401, A402, |
| | A403, A404, A41, A410, A412, A413, A416, A417, A420, A421, A423, A426, A43, A431, A432, A433, |
| | A434, A435, A436, A437, A438, A441, A443, A444, A445, A447, A45, A450, A451, A452, A454, A455, |
| | A456, A457, A458, A46, A463, A466, A467, A468, A469, A47, A471, A472, A473, A474, A475, A476, |
| | A477, A478, A48, A480, A484, A485, A486, A487, A488, A489, A490, A492, A493, A495, A502, A505, |
| | A506, A508, A509, A51, A510, A511, A512, A513, A514, A515, A518, A520, A521, A522, A523, A525, |
| | A527, A528, A530, A531, A532, A533, A534, A535, A536, A538, A540, A541, A542, A543, A545, A547, |
| | A549, A550, A551, A552, A556, A557, A559, A560, A562, A563, A564, A566, A567, A568, A569, |
| | A570, A571, A574, A576, A580, A581, A582, A583, A585, A586, A587, A588, A589, A591, A593, A595, |
| | A596, A597, A598, A600, A601, A602, A603, A608, A610, A611, A614, A616, A62, A64, A65, A66, A67, |
| | A69, A76, A78, A79, A8, A80, A81, A83, A84, A85, A87, A88, A89, A9, A90, A91, A93, A94 |
| ++++ | A105, A106, A107, A108, A109, A110, A112, A113, A115, A116, A118, A12, A137, A138, A141, A142, |
| | A143, A144, A152, A175, A176, A177, A178, A178, A179, A179, A180, A181, A182, A183, A185, A203, |
| | A207, A208, A210, A213, A214, A220, A221, A222, A225, A225, A236, A237, A238, A239, A244, |
| | A245, A246, A253, A256, A258, A261, A271, A272, A281, A282, A283, A292, A299, A3, A300, A305, |
| | A307, A308, A317, A322, A339, A34, A345, A355, A359, A36, A366, A388, A389, A398, A4, A42, A425, |
| | A428, A439, A44, A440, A442, A446, A448, A449, A453, A460, A461, A462, A464, A470, A479, A481, |
| | A482, A49, A496, A497, A499, A50, A504, A516, A517, A519, A52, A524, A53, A539, A54, A54, A544, |
| | A548, A55, A553, A56, A57, A572, A575, A579, A58, A59, A592, A599, A60, A604, A609, A61, A612, |
| | A63, A70, A75, A82, A86, A92, A96, A97, A98, A99, A613 |
| +++++ | A103, A103, A104, A104, A116, A117, A145, A150, A153, A154, A172, A186, A187, A188, A189, A190, |
| | A191, A192, A193, A194, A195, A196, A197, A198, A199, A200, A206, A212, A234, A235, A253, A327, |
| | A328, A340, A341, A429, A430, A77 |

TABLE 10
KRAS G13C FRET data

| IC50* | Examples |
|---|---|
| + | A381, A325, A501, A594 |
| ++ | A1, A10, A100, A101, A102, A111, A114, A121, A123, A124, A125, A126, A127, A128, A129, A130, |
| | A131, A132, A133, A134, A135, A139, A140, A146, A147, A148, A149, A151, A155, A156, A159, A160, |
| | A162, A164, A165, A166, A168, A169, A171, A184, A201, A202, A204, A21, A211, A215, A216, A217, |
| | A218, A219, A223, A224, A226, A227, A227, A228, A229, A23, A230, A231, A233, A240, A241, A242, |
| | A243, A247, A248, A248, A249, A25, A250, A251, A252, A252, A255, A266, A27, A275, A277, A3, A31, |
| | A323, A324, A342, A346, A347, A348, A349, A351, A364, A365, A370, A371, A377, A378, A379, |
| | A380, A384, A385, A386, A391, A392, A393, A394, A395, A396, A397, A4, A405, A406, A407, A408, |
| | A409, A41, A410, A413, A414, A415, A418, A419, A420, A421, A422, A424, A426, A427, A432, A459, |
| | A465, A500, A507, A509, A526, A529, A546, A554, A555, A562, A573, A577, A578, A584, A605, |
| | A607, A615, A616, A67, A68, A78, A87, A88, A89, A95 |
| +++ | A105, A106, A107, A109, A11, A117, A118, A119, A12, A120, A122, A13, A136, A138, A14, A142, |
| | A144, A15, A157, A158, A16, A161, A163, A167, A17, A170, A174, A175, A176, A177, A178, A178, |
| | A179, A179, A18, A180, A181, A182, A183, A185, A19, A2, A20, A205, A207, A208, A209, A214, A22, |
| | A225, A225, A232, A236, A236, A24, A253, A254, A254, A256, A257, A258, A259, A26, A262, A264, |
| | A265, A267, A268, A270, A273, A274, A276, A278, A279, A28, A280, A284, A285, A286, A287, A288, |
| | A289, A290, A291, A293, A294, A295, A296, A297, A298, A30, A302, A303, A304, A306, A309, A310, |
| | A311, A312, A313, A314, A315, A316, A318, A319, A32, A320, A321, A322, A329, A33, A330, A331, |
| | A332, A333, A334, A335, A336, A337, A338, A343, A344, A345, A35, A350, A352, A353, A354, A355, |
| | A356, A357, A358, A359, A360, A361, A362, A363, A366, A367, A368, A369, A37, A372, A373, A374, |
| | A375, A376, A382, A383, A387, A388, A39, A398, A399, A4, A40, A400, A401, A402, A403, A404, |
| | A411, A412, A416, A417, A423, A425, A43, A431, A433, A434, A435, A436, A437, A438, A439, A441, |
| | A443, A444, A445, A447, A449, A45, A450, A451, A452, A454, A456, A457, A458, A46, A462, A463, |
| | A466, A467, A468, A469, A47, A470, A471, A472, A473, A474, A475, A476, A477, A478, A48, A480, |
| | A483, A484, A485, A486, A487, A488, A489, A490, A491, A492, A493, A494, A495, A497, A498, A5, |
| | A502, A503, A504, A505, A506, A508, A51, A510, A511, A512, A513, A514, A515, A518, A520, A521, |
| | A522, A523, A524, A525, A528, A530, A531, A532, A533, A534, A535, A536, A537, A538, A540, A541, |
| | A542, A543, A544, A545, A547, A548, A549, A55, A550, A551, A552, A553, A556, A557, A558, A559, |
| | A560, A561, A563, A564, A565, A566, A567, A568, A569, A570, A571, A574, A576, A579, A580, |
| | A581, A582, A583, A585, A586, A587, A588, A589, A590, A591, A592, A593, A595, A596, A597, A598, |
| | A6, A600, A601, A602, A603, A606, A608, A610, A611, A614, A62, A65, A66, A69, A7, A75, A76, A79, |
| | A8, A80, A81, A82, A83, A84, A85, A86, A87, A9, A90, A91, A92, A93, A94, A96, A98, A99 |
| ++++ | A104, A104, A108, A110, A112, A113, A115, A116, A12, A137, A141, A143, A150, A152, A172, A173, |
| | A188, A199, A203, A206, A210, A212, A213, A220, A221, A222, A234, A235, A237, A238, A239, A244, |
| | A245, A246, A253, A260, A261, A271, A272, A281, A282, A283, A29, A292, A299, A3, A300, A301, |
| | A305, A307, A308, A317, A326, A339, A34, A36, A36, A38, A389, A42, A428, A44, A440, A442, A446, |
| | A448, A453, A455, A460, A461, A464, A479, A481, A482, A49, A496, A499, A50, A516, A517, A519, |
| | A52, A527, A53, A539, A54, A54, A56, A57, A572, A575, A58, A599, A604, A609, A61, A612, A63, |
| | A64, A70, A77, A97, A613 |
| +++++ | A103, A103, A116, A117, A145, A153, A154, A186, A187, A189, A190, A191, A192, A193, A194, A195, |
| | A196, A197, A198, A200, A327, A328, A340, A341, A429, A430, A59, A60 |

TABLE 11
KRAS G12V FRET data

| IC50* | Examples |
|---|---|
| + | A325 |
| ++ | A1, A11, A114, A117, A121, A135, A139, A140, A146, A147, A156, A159, A160, A162, A164, A165, A2, |
| | A201, A202, A204, A211, A218, A219, A223, A224, A230, A233, A247, A248, A249, A250, A252, A264, |
| | A265, A266, A275, A277, A278, A279, A3, A323, A342, A347, A348, A349, A365, A370, A371, A377, |
| | A378, A379, A380, A385, A391, A396, A399, A405, A407, A415, A422, A423, A424, A427, A454, |
| | A465, A477, A487, A5, A500, A501, A507, A526, A529, A546, A554, A555, A562, A577, A578, A584, |
| | A594, A605, A607, A615, A95 |
| +++ | A10, A100, A102, A12, A120, A123, A124, A125, A126, A127, A128, A129, A13, A131, A132, A136, |
| | A137, A138, A14, A148, A149, A15, A151, A155, A157, A158, A16, A161, A166, A167, A168, A17, |
| | A170, A171, A174, A176, A18, A184, A19, A20, A205, A209, A21, A215, A217, A22, A226, A227, A228, A229, |
| | A231, A232, A236, A24, A240, A241, A242, A243, A248, A25, A251, A252, A254, A254, A255, A257, |
| | A26, A262, A267, A268, A270, A273, A274, A28, A280, A285, A286, A287, A288, A289, A29, A290, |
| | A291, A293, A294, A295, A296, A297, A298, A30, A301, A302, A303, A304, A306, A309, A310, A311, |
| | A312, A313, A314, A315, A316, A318, A319, A320, A321, A322, A324, A326, A329, A33, A330, |
| | A331, A332, A333, A334, A335, A336, A337, A338, A343, A344, A345, A346, A35, A350, A351, A352, |
| | A353, A354, A355, A356, A357, A358, A359, A36, A36, A360, A361, A362, A363, A364, A366, A367, |
| | A368, A369, A37, A372, A373, A374, A375, A376, A38, A381, A382, A383, A384, A386, A387, A39, |
| | A392, A393, A394, A395, A397, A4, A40, A400, A401, A402, A403, A406, A408, A409, A410, A411, |
| | A412, A413, A414, A416, A418, A419, A420, A425, A426, A43, A431, A433, A436, A437, A438, A44, |
| | A441, A443, A444, A445, A450, A451, A452, A456, A457, A458, A459, A463, A466, A467, A471, A472, |
| | A474, A475, A476, A478, A480, A483, A484, A485, A486, A488, A489, A49, A490, A491, A492, A493, |
| | A494, A495, A496, A498, A503, A505, A506, A509, A510, A511, A512, A513, A514, A515, A520, A521, |
| | A522, A523, A525, A528, A530, A531, A532, A533, A534, A535, A536, A537, A538, A540, A541, |
| | A543, A545, A547, A548, A549, A550, A551, A552, A553, A557, A558, A559, A560, A561, A564, A565, |
| | A566, A567, A568, A569, A570, A571, A573, A574, A576, A579, A580, A581, A582, A583, A585, A586, |
| | A589, A590, A591, A593, A595, A596, A597, A598, A6, A600, A601, A602, A603, A606, A608, A610, |
| | A614, A616, A62, A64, A67, A68, A7, A76, A78, A8, A83, A87, A89, A9, A90, A91, A93, A94 |
| ++++ | A101, A105, A106, A111, A112, A113, A115, A116, A117, A12, A122, A130, A133, A134, A141, A142, |
| | A143, A144, A152, A163, A169, A173, A177, A178, A178, A179, A179, A180, A181, A182, A183, A185, |
| | A199, A203, A207, A208, A210, A212, A214, A216, A220, A221, A225, A225, A227, A23, A236, A237, |
| | A238, A239, A244, A245, A246, A253, A253, A256, A258, A259, A260, A261, A27, A271, A276, |
| | A281, A282, A283, A284, A292, A299, A3, A300, A305, A307, A308, A31, A317, A32, A339, A34, A388, |
| | A389, A398, A404, A41, A417, A42, A421, A428, A432, A434, A435, A439, A440, A442, A447, A449, |
| | A453, A455, A46, A460, A461, A462, A464, A468, A469, A470, A473, A479, A48, A481, A482, A497, |
| | A499, A50, A502, A504, A508, A51, A516, A517, A518, A519, A52, A524, A527, A53, A539, A54, A54, |
| | A542, A544, A55, A556, A56, A563, A57, A572, A575, A58, A587, A588, A59, A592, A599, A60, A604, |
| | A609, A61, A611, A612, A63, A65, A66, A69, A75, A79, A80, A81, A82, A84, A85, A86, A87, A88, A92, |
| | A96, A97, A98, A99, A613 |
| +++++ | A103, A103, A104, A104, A107, A108, A109, A110, A116, A118, A119, A145, A150, A153, A154, A172, |
| | A175, A186, A187, A188, A189, A190, A191, A192, A193, A194, A195, A196, A197, A198, A200, A206, |
| | A213, A222, A234, A235, A272, A327, A328, A340, A341, A4, A429, A430, A446, A448, A45, A47, A70, A77 |

TABLE 12
KRAS WT FRET data

| IC50* | Examples |
|---|---|
| + | A594 |
| ++ | A1, A10, A100, A111, A114, A121, A124, A125, A126, A127, A128, A129, A130, A131, A135, |
| | A139, A140, A146, A147, A148, A149, A151, A155, A156, A157, A159, A160, A162, A164, A165, A166, A168, |
| | A2, A202, A204, A211, A216, A217, A218, A219, A223, A224, A227, A228, A23, A230, A231, |
| | A241, A242, A243, A247, A248, A249, A252, A252, A274, A275, A277, A278, A287, A290, A291, A298, A3, |
| | A306, A312, A323, A325, A333, A335, A336, A342, A346, A347, A348, A349, A351, A363, A365, A370, |
| | A371, A372, A373, A374, A377, A378, A379, A380, A381, A385, A386, A391, A392, A393, A395, A396, |
| | A397, A4, A400, A402, A405, A406, A407, A409, A410, A411, A412, A413, A415, A418, A419, A422, |
| | A424, A426, A427, A443, A454, A459, A465, A475, A483, A487, A491, A493, A494, A498, A500, A501, |
| | A503, A505, A507, A526, A528, A529, A536, A537, A545, A546, A554, A555, A558, A560, A561, |
| | A562, A565, A571, A573, A574, A577, A578, A584, A590, A596, A600, A605, A606, A607, |
| | A615, A616, A87, A95 |
| +++ | A101, A102, A106, A11, A117, A12, A120, A122, A123, A13, A132, A133, A134, A136, A137, A138, A14, |
| | A144, A15, A158, A16, A161, A163, A167, A169, A17, A170, A171, A173, A174, A176, A178, A179, |
| | A179, A18, A184, A19, A20, A201, A205, A208, A209, A21, A210, A215, A22, A226, A227, A229, A232, |
| | A233, A236, A24, A240, A248, A25, A250, A251, A254, A254, A255, A257, A259, A26, A260, A262, |
| | A264, A265, A266, A267, A268, A27, A270, A273, A276, A279, A28, A280, A283, A284, A285, A286, |
| | A288, A289, A292, A293, A294, A295, A296, A297, A299, A30, A301, A302, A303, A304, A308, A309, |
| | A31, A310, A311, A313, A314, A315, A316, A318, A319, A32, A320, A321, A322, A324, A326, A329, |
| | A330, A331, A332, A334, A337, A338, A339, A343, A344, A345, A35, A350, A352, A353, A354, A355, |
| | A356, A357, A358, A360, A361, A362, A364, A367, A368, A369, A37, A375, A376, A38, A382, A383, |
| | A384, A387, A39, A394, A399, A40, A401, A403, A404, A408, A41, A414, A416, A417, A420, A421, |
| | A423, A43, A432, A433, A434, A435, A436, A437, A438, A439, A44, A440, A441, A444, A445, A447, |
| | A449, A45, A450, A451, A452, A455, A456, A457, A458, A46, A461, A463, A466, A467, A468, A469, |
| | A47, A471, A472, A473, A474, A476, A477, A478, A48, A480, A484, A485, A486, A488, A489, A49, |
| | A490, A492, A495, A496, A5, A50, A502, A504, A506, A508, A509, A51, A510, A511, A512, A513, |
| | A514, A515, A518, A519, A520, A521, A522, A523, A524, A525, A527, A530, A531, A532, A533, A534, |
| | A535, A538, A540, A541, A542, A543, A547, A548, A549, A550, A551, A552, A553, A556, A557, A559, |
| | A563, A564, A566, A567, A568, A569, A570, A576, A579, A580, A581, A582, A583, A585, A586, |
| | A587, A588, A589, A591, A593, A595, A597, A598, A6, A601, A602, A603, A608, A610, A611, A614, |
| | A62, A64, A65, A66, A67, A68, A7, A76, A78, A79, A8, A80, A81, A82, A83, A84, A85, A87, A88, A89, A9, |
| | A90, A91, A93, A94, A99 |
| ++++ | A105, A107, A108, A109, A110, A112, A113, A115, A116, A118, A119, A12, A141, A142, A143, |
| | A150, A152, A175, A177, A178, A180, A181, A182, A183, A185, A199, A203, A206, A207, A213, |
| | A214, A220, A221, A222, A225, A225, A234, A235, A236, A237, A238, A239, A244, A245, A246, A253, A253, |
| | A256, A258, A261, A271, A272, A281, A282, A29, A3, A300, A305, A307, A317, A33, A34, A359, A36, |
| | A36, A366, A388, A389, A398, A4, A42, A425, A428, A429, A431, A442, A446, A448, A453, A460, |
| | A462, A464, A470, A479, A481, A482, A497, A499, A516, A517, A52, A53, A539, A54, A54, A544, |
| | A55, A56, A57, A572, A575, A58, A59, A592, A599, A60, A604, A609, A61, A612, A63, A69, A70, |
| | A75, A86, A92, A96, A97, A98, A613 |
| +++++ | A103, A103, A104, A104, A116, A117, A145, A153, A154, A172, A186, A187, A188, A189, A190, A191, |
| | A192, A193, A194, A195, A196, A197, A198, A200, A212, A327, A328, A340, A341, A430, A77 |

TABLE 13
KRAS G13D FRET data

| IC50* | Examples (Example A55 not tested) |
|---|---|
| + | None |
| ++ | A1, A10, A100, A111, A114, A121, A124, A125, A127, A128, A129, A130, A131, A133, A134, A135, |
| | A139, A140, A148, A151, A155, A159, A162, A163, A164, A165, A169, A2, A202, A204, A211, A216, |
| | A217, A223, A224, A227, A227, A228, A229, A23, A231, A242, A243, A247, A248, A249, A251, A252, |
| | A255, A27, A275, A277, A3, A323, A325, A342, A347, A348, A349, A365, A370, A371, A378, A379, |
| | A380, A381, A385, A386, A391, A392, A393, A395, A396, A397, A4, A405, A407, A409, A41, A415, |
| | A419, A424, A426, A427, A432, A45, A459, A501, A507, A529, A546, A558, A573, A577, A578, |
| | A584, A594, A605, A607, A615, A67, A87, A88, A95 |
| +++ | A101, A102, A11, A117, A119, A12, A120, A122, A123, A126, A13, A132, A136, A14, A146, A147, |
| | A149, A15, A156, A157, A158, A16, A160, A161, A166, A167, A168, A17, A170, A171, A174, A176, |
| | A179, A18, A184, A19, A20, A201, A205, A209, A21, A215, A218, A219, A226, A230, A232, A233, A236, A24, |
| | A240, A241, A248, A25, A250, A252, A254, A254, A257, A259, A26, A262, A264, A265, A266, A267, |
| | A268, A270, A273, A274, A278, A279, A28, A280, A284, A285, A286, A287, A288, A289, A290, A291, |
| | A293, A294, A295, A298, A302, A303, A304, A306, A309, A31, A310, A311, A312, A313, A314, |
| | A315, A316, A318, A319, A32, A321, A324, A329, A330, A332, A333, A334, A335, A336, A337, A338, |
| | A343, A345, A346, A35, A350, A351, A352, A353, A356, A357, A358, A361, A363, A364, A369, |
| | A372, A373, A374, A375, A376, A377, A38, A382, A383, A384, A387, A39, A394, A399, A4, A40, A400, A401, |
| | A402, A403, A404, A406, A408, A410, A411, A412, A413, A414, A416, A417, A418, A420, A421, |
| | A422, A423, A43, A433, A434, A435, A436, A437, A438, A441, A442, A443, A444, A445, A447, A450, |
| | A452, A454, A457, A458, A46, A463, A465, A466, A467, A468, A469, A47, A471, A473, A475, A476, |
| | A477, A478, A48, A480, A483, A484, A485, A486, A487, A488, A489, A491, A492, A493, A494, A498, |
| | A5, A500, A502, A503, A504, A505, A506, A509, A51, A510, A512, A513, A514, A515, A518, A520, |
| | A523, A525, A526, A528, A531, A532, A533, A534, A535, A536, A537, A538, A540, A542, A543, A545, |
| | A549, A550, A551, A552, A554, A555, A557, A559, A560, A561, A562, A563, A564, A565, A566, A567, |
| | A568, A569, A570, A571, A574, A576, A580, A581, A582, A583, A586, A587, A588, A589, A590, |
| | A591, A593, A595, A596, A597, A598, A6, A600, A601, A602, A603, A606, A608, A610, A611, |
| | A614, A616, A62, A64, A65, A66, A68, A7, A76, A78, A79, A8, A80, A81, A83, A84, A85, |
| | A87, A89, A9, A90, A93, A94 |
| ++++ | A105, A106, A107, A108, A109, A110, A112, A113, A115, A116, A118, A12, A137, A138, A141, A142, |
| | A143, A144, A152, A173, A175, A177, A178, A178, A179, A180, A181, A182, A183, A185, A199, A203, |
| | A206, A207, A208, A210, A213, A214, A22, A222, A225, A225, A236, A238, A239, A246, A253, A256, |
| | A258, A260, A261, A272, A276, A281, A282, A283, A29, A292, A296, A297, A299, A30, A300,A301, |
| | A305, A307, A308, A317, A320, A322, A326, A33, A331, A339, A34, A344, A354, A355, A359, A36, A36, |
| | A360, A362, A366, A367, A368, A37, A388, A389, A398, A42, A425, A431, A439, A44, A440, |
| | A446, A448, A449, A451, A455, A456, A460, A461, A462, A464, A470, A472, A474, A479, A481, |
| | A482, A49, A490, A495, A496, A497, A499, A50, A508, A511, A516, A517, A519, A52, A521, A522, A524, |
| | A527, A53, A530, A539, A54, A541, A544, A547, A548, A553, A556, A56, A57, A572, A575, A579, A58, |
| | A585, A59, A592, A599, A604, A609, A61, A612, A63, A69, A70, A75, A77, A82, A86, A91, A92, A96, |
| | A97, A98, A99, A613 |
| +++++ | A103, A103, A104, A104, A116, A117, A145, A150, A153, A154, A172, A186, A187, A188, A189, A190, |
| | A191, A192, A193, A194, A195, A196, A197, A198, A200, A212, A220, A221, A234, A235, A237, A244, |
| | A245, A253, A271, A3, A327, A328, A340, A341, A428, A429, A430, A453, A60 |

TABLE 15
NRAS G12C FRET data

| IC50* | Examples |
|---|---|
| + | A323, A325, A501, A577, A578, A594 |
| ++ | A1, A10, A100, A11, A114, A120, A121, A125, A127, A128, A129, A131, A135, A136, A139, A140, |
| | A146, A147, A148, A151, A156, A157, A159, A160, A162, A164, A165, A166, A168, A2, A201, A202, |
| | A204, A205, A211, A217, A218, A219, A223, A224, A228, A229, A230, A231, A233, A240, A242, A243, |
| | A247, A248, A248, A249, A250, A252, A252, A264, A265, A266, A267, A268, A273, A274, A275, A277, |
| | A278, A279, A280, A285, A288, A289, A290, A291, A298, A3, A302, A303, A304, A306, A309, A310, |
| | A312, A313, A316, A319, A321, A330, A333, A334, A335, A336, A338, A342, A343, A346, A347, A348, |
| | A349, A350, A351, A353, A356, A357, A358, A363, A364, A365, A370, A371, A372, A373, A374, |
| | A376, A377, A378, A379, A380, A381, A382, A383, A384, A385, A386, A387, A391, A392, A393, A395, |
| | A396, A397, A399, A4, A400, A401, A402, A405, A406, A407, A408, A409, A411, A413, A414, A415, |
| | A418, A419, A422, A424, A426, A427, A432, A436, A438, A443, A444, A450, A452, A454, A457, A458, |
| | A459, A465, A466, A467, A471, A475, A476, A477, A478, A483, A484, A487, A488, A489, A491, |
| | A493, A494, A498, A5, A500, A503, A505, A507, A509, A510, A514, A515, A523, A526, A528, A529, |
| | A531, A533, A534, A536, A537, A538, A540, A543, A545, A546, A549, A550, A551, A552, A554, A555, |
| | A557, A558, A559, A560, A561, A562, A565, A567, A569, A571, A573, A574, A576, A581, A582, A583, |
| | A584, A590, A591, A593, A595, A596, A598, A6, A600, A601, A605, A606, A607, A608, A614, A615, |
| | A616, A8, A87, A95 |
| +++ | A101, A102, A106, A111, A117, A122, A123, A124, A126, A13, A130, A132, A133, A134, A137, A138, |
| | A14, A149, A15, A155, A158, A16, A161, A163, A167, A169, A17, A170, A171, A173, A174, A176, |
| | A18, A184, A19, A20, A209, A21, A210, A215, A216, A226, A227, A227, A23, A232, A236, A24, |
| | A241, A25, A251, A254, A254, A255, A257, A259, A26, A260, A262, A27, A270, A276, A28, A282, A283, A284, |
| | A286, A287, A29, A292, A293, A294, A295, A296, A297, A299, A30, A301, A305, A308, A31, A311, |
| | A314, A315, A318, A32, A320, A322, A324, A326, A329, A331, A332, A337, A339, A344, A345, A35, |
| | A352, A354, A355, A359, A36, A36, A360, A361, A362, A366, A367, A368, A369, A37, A375, A38, A389, |
| | A39, A394, A40, A403, A404, A41, A410, A412, A416, A417, A420, A421, A423, A43, A431, A433, |
| | A434, A435, A437, A439, A44, A440, A441, A445, A447, A449, A451, A453, A455, A456, A46, A460, A461, |
| | A462, A463, A468, A469, A47, A472, A473, A474, A479, A480, A481, A482, A485, A486, A490, |
| | A492, A495, A496, A497, A499, A502, A504, A506, A508, A511, A512, A513, A518, A519, A520, A521, |
| | A522, A524, A525, A527, A530, A532, A535, A539, A541, A542, A544, A547, A548, A553, A556, A563, |
| | A564, A566, A568, A570, A579, A580, A585, A586, A587, A588, A589, A592, A597, A602, A603, |
| | A609, A610, A611, A612, A62, A64, A65, A66, A67, A68, A76, A78, A79, A80, A81, A83, A85, A87, |
| | A88, A89, A9, A90, A91, A93, A94, A96, A97, A98, A99 |
| ++++ | A105, A107, A108, A109, A110, A112, A113, A115, A116, A117, A118, A119, A12, A141, A142, A143, |
| | A144, A150, A152, A175, A177, A178, A178, A179, A179, A180, A181, A182, A183, A185, A199, A203, |
| | A206, A207, A208, A212, A213, A214, A220, A221, A225, A225, A235, A236, A237, A238, A239, |
| | A244, A245, A246, A253, A253, A256, A258, A261, A271, A272, A281, A3, A300, A307, A317, A388, |
| | A398, A4, A42, A425, A428, A429, A442, A446, A448, A45, A464, A470, A49, A50, A51, A516, A517, |
| | A52, A53, A54, A54, A56, A57, A572, A575, A58, A59, A599, A60, A604, A61, A63, A75, A82, A84, |
| | A86, A92, A613 |
| +++++ | A103, A103, A104, A104, A116, A145, A153, A154, A172, A186, A187, A188, A189, A190, A191, A192, |
| | A193, A194, A195, A196, A197, A198, A200, A222, A234, A327, A328, A340, A341, A430, A77 |

TABLE 14
KRAS Q61H FRET data

| IC50* | Examples |
|---|---|
| + | A159, A275, A415, A501, A546, A577, A594, A605 |
| ++ | A102, A124, A136, A174, A204, A205, A226, A230, A236, A24, A25, A250, A266, A268, A273, A274, |
| | A277, A278, A279, A280, A285, A287, A289, A290, A291, A3, A302, A304, A306, A309, A310, A312, |
| | A316, A324, A334, A335, A346, A349, A356, A358, A37, A372, A373, A374, A376, A38, A382, A383, |
| | A387, A396, A399, A400, A401, A405, A406, A409, A422, A424, A426, A434, A438, A443, A444, |
| | A450, A454, A457, A459, A465, A466, A467, A468, A471, A474, A475, A477, A478, A480, A483, A484, |
| | A485, A487, A488, A491, A492, A493, A494, A498, A500, A502, A503, A505, A507, A509, A510, A514, |
| | A515, A520, A523, A526, A528, A529, A530, A531, A533, A534, A536, A537, A538, A540, A543, A545, |
| | A549, A550, A551, A552, A554, A555, A557, A558, A559, A560, A561, A562, A563, A564, A565, A566, |
| | A567, A569, A571, A573, A574, A578, A581, A582, A583, A584, A589, A590, A591, A593, A595, |
| | A596, A600, A601, A606, A607, A608, A610, A614, A615, A616, A89 |
| +++ | A138, A144, A152, A163, A170, A176, A177, A178, A178, A179, A179, A180, A181, A207, A208, A210, |
| | A225, A225, A253, A264, A265, A267, A270, A271, A272, A276, A281, A282, A283, A284, A286, A293, |
| | A297, A299, A305, A315, A320, A321, A331, A337, A344, A354, A357, A359, A360, A361, A362, |
| | A366, A367, A368, A369, A375, A388, A389, A423, A433, A435, A436, A437, A439, A440, A441, A442, |
| | A445, A447, A449, A451, A452, A453, A455, A456, A458, A460, A461, A462, A463, A464, A469, A470, |
| | A472, A473, A476, A479, A481, A482, A486, A489, A490, A495, A496, A497, A499, A504, A506, |
| | A508, A511, A512, A513, A518, A519, A521, A522, A524, A525, A527, A532, A535, A54, A541, A542, |
| | A544, A547, A548, A553, A556, A568, A57, A570, A572, A576, A579, A580, A585, A586, A587, A588, |
| | A592, A597, A598, A599, A602, A603, A604, A609, A61, A611, A612, A99 |
| ++++ | A104, A145, A150, A220, A234, A237, A446, A448, A516, A517, A539, A575, A613 |
| +++++ | A154, A186, A189, A191, A328, A340 |

TABLE 16
NRAS WT FRET data

| IC50* | Examples |
|---|---|
| + | A501, A577, A594 |
| ++ | A124, A136, A159, A204, A230, A25, A250, A273, A274, A275, A277, A278, A279, A287, A290, A291, |
| | A3, A304, A306, A309, A310, A312, A335, A346, A349, A356, A372, A373, A374, A387, A396, A399, |
| | A400, A405, A406, A409, A415, A422, A424, A426, A434, A438, A443, A450, A454, A457, A459, A465, |
| | A466, A471, A475, A477, A478, A483, A484, A487, A491, A493, A494, A498, A500, A503, A505, A507, |
| | A526, A528, A529, A531, A533, A536, A537, A545, A546, A554, A555, A558, A560, A561, A562, |
| | A565, A569, A571, A573, A574, A578, A584, A590, A591, A596, A600, A605, A606, A607, A615 |
| +++ | A102, A138, A144, A163, A170, A174, A176, A178, A179, A179, A205, A208, A210, A225, A226, A236, |
| | A24, A253, A264, A265, A266, A267, A268, A270, A276, A280, A283, A284, A285, A286, A289, A293, |
| | A297, A299, A302, A305, A315, A316, A320, A321, A324, A331, A334, A337, A344, A354, A357, |
| | A358, A359, A360, A361, A362, A366, A367, A368, A369, A37, A375, A376, A38, A382, A383, A401, |
| | A423, A433, A435, A436, A437, A439, A440, A441, A444, A445, A447, A449, A451, A452, A455, A456, |
| | A458, A463, A467, A468, A469, A472, A473, A474, A476, A479, A480, A481, A485, A486, A488, |
| | A489, A490, A492, A495, A496, A497, A502, A504, A506, A508, A509, A510, A511, A512, A513, A514, |
| | A515, A518, A519, A520, A521, A522, A523, A524, A525, A527, A530, A532, A534, A535, A538, A540, |
| | A541, A542, A543, A547, A548, A549, A550, A551, A552, A553, A556, A557, A559, A563, A564, |
| | A566, A567, A568, A570, A576, A579, A580, A581, A582, A583, A585, A586, A587, A588, A589, A593, |
| | A595, A597, A598, A601, A602, A603, A608, A610, A611, A612, A614, A616, A89, A99 |
| ++++ | A104, A150, A152, A177, A178, A180, A181, A207, A220, A225, A234, A237, A271, A272, A281, A282, |
| | A388, A389, A442, A446, A448, A453, A460, A461, A462, A464, A470, A482, A499, A516, A517, A539, |
| | A54, A544, A57, A572, A575, A592, A599, A604, A609, A61, A613 |
| +++++ | A145, A154, A186, A189, A191, A328, A340 |

TABLE 17
NRAS Q61K FRET data

| IC50* | Examples |
|---|---|
| + | A275 |
| ++ | A136, A159, A170, A205, A266, A268, A277, A278, A279, A280, A285, A289, A290, A291, A302, A304, |
| | A306, A309, A310, A312, A316, A334, A335, A337, A344, A349, A356, A358, A372, A373, A374, A376, |
| | A382, A383, A387, A396, A399, A400, A401, A405, A409, A415, A422, A443, A444, A445, A450, |
| | A451, A452, A454, A457, A458, A459, A465, A466, A467, A471, A475, A477, A478, A483, A484, A485, |
| | A486, A487, A488, A489, A491, A493, A494, A500, A501, A503, A505, A510, A511, A512, A514, A515, |
| | A520, A522, A523, A526, A528, A529, A530, A531, A532, A533, A534, A536, A537, A538, A540, |
| | A543, A546, A549, A550, A551, A552, A555, A557, A560, A561, A565, A566, A567, A569, A571, A573, |
| | A574, A576, A577, A578, A580, A581, A582, A584, A590, A591, A594, A595, A596, A598, A600, |
| | A601, A606, A607, A608, A614 |
| +++ | A102, A124, A138, A144, A152, A174, A176, A204, A208, A210, A226, A230, A236, A24, A25, A250, |
| | A264, A265, A267, A270, A271, A273, A274, A276, A281, A283, A284, A286, A287, A293, A297, A299, |
| | A3, A305, A315, A320, A321, A324, A331, A346, A354, A357, A359, A360, A361, A362, A366, A367, |
| | A368, A369, A375, A38, A389, A406, A424, A426, A433, A434, A436, A437, A438, A439, A440, A441, |
| | A447, A449, A453, A455, A456, A460, A461, A462, A468, A469, A472, A474, A476, A479, A480, |
| | A481, A482, A490, A492, A495, A496, A497, A498, A499, A502, A506, A507, A509, A513, A521, A524, |
| | A525, A527, A535, A541, A542, A544, A545, A547, A548, A553, A554, A556, A558, A559, A562, |
| | A563, A564, A568, A570, A579, A583, A585, A586, A587, A588, A589, A592, A593, A597, A602, A603, |
| | A604, A605, A609, A610, A611, A612, A613, A615, A616, A89 |
| ++++ | A104, A150, A163, A177, A178, A178, A179, A179, A180, A181, A207, A220, A225, A225, A234, A237, |
| | A253, A272, A282, A37, A388, A423, A435, A442, A446, A448, A463, A464, A470, A473, A504, A508, |
| | A516, A517, A518, A519, A539, A54, A57, A572, A575, A599, A61, A99 |
| +++++ | A145, A154, A186, A189, A191, A328, A340 |

TABLE 18
NRAS Q61R FRET data

| IC50* | Examples |
|---|---|
| + | A577, A594 |
| ++ | A136, A159, A275, A277, A278, A279, A291, A304, A306, A309, A310, A312, A335, A349, A356, A372, |
| | A373, A374, A396, A399, A400, A415, A422, A450, A454, A459, A465, A466, A475, A477, A483, A487, |
| | A494, A500, A501, A503, A505, A520, A526, A528, A529, A536, A546, A555, A560, A561, A562, |
| | A565, A578, A583, A584, A593, A601, A606, A607 |
| +++ | A102, A138, A170, A174, A204, A205, A210, A226, A230, A24, A25, A250, A264, A265, A266, A267, |
| | A268, A270, A273, A274, A276, A280, A283, A285, A286, A287, A289, A290, A293, A297, A299, A3, |
| | A302, A305, A316, A321, A334, A337, A344, A346, A354, A357, A358, A361, A369, A375, A376, |
| | A38, A382, A383, A387, A401, A405, A406, A409, A423, A424, A426, A433, A434, A436, A437, A438, |
| | A439, A440, A441, A443, A444, A445, A449, A451, A452, A453, A455, A456, A457, A458, A461, A467, |
| | A468, A471, A472, A474, A476, A478, A480, A484, A485, A486, A488, A489, A490, A491, A492, A493, |
| | A495, A498, A499, A502, A506, A507, A509, A510, A511, A512, A513, A514, A515, A521, A522, |
| | A523, A525, A527, A530, A531, A532, A533, A534, A535, A537, A538, A540, A541, A542, A543, A545, |
| | A547, A548, A549, A550, A551, A552, A553, A554, A556, A557, A558, A559, A563, A564, A566, A567, |
| | A568, A569, A570, A571, A573, A574, A576, A579, A580, A581, A582, A585, A586, A587, A589, |
| | A590, A591, A595, A596, A597, A598, A600, A602, A603, A605, A608, A609, A610, A611, A614, A615, |
| | A616, A89 |
| ++++ | A124, A144, A152, A163, A176, A177, A178, A179, A179, A180, A207, A208, A220, A225, A225, |
| | A236, A237, A253, A271, A272, A281, A282, A284, A315, A320, A324, A331, A359, A360, A362, |
| | A366, A367, A368, A37, A388, A389, A435, A442, A447, A448, A460, A462, A463, A464, A469, A470, A473, |
| | A479, A481, A482, A496, A497, A504, A508, A516, A517, A518, A519, A524, A539, A54, A544, A57, |
| | A572, A575, A588, A592, A599, A604, A61, A612, A99, A613 |
| +++++ | A104, A145, A150, A154, A178, A181, A186, A189, A191, A234, A328, A340, A446 |

### In Vitro Cell Proliferation Panels

Potency for inhibition of cell growth was assessed at CrownBio using standard methods. Briefly, cell lines were cultured in appropriate medium, and then plated in 3D methylcellulose. Inhibition of cell growth was determined by CellTiter-Glo® after 5 days of culture with increasing concentrations of compounds. Compound potency was reported as the 50% inhibition concentration (absolute IC50). The assay took place over 7 days. On day 1, cells in 2D culture were harvested during logarithmic growth and suspended in culture medium at 1×105 cells/mi. Higher or lower cell densities were used for some cell lines based on prior optimization. 3.5 ml of cell suspension was mixed with 6.5% growth medium with 1% methylcellulose, resulting in a cell suspension in 0.65% methylcellulose. 90 μl of this suspension was distributed in the wells of 2 96-well plates. One plate was used for day 0 reading and 1 plate was used for the end-point experiment. Plates were incubated overnight at 37 C with 5% CO2. On day 2, one plate (for t0 reading) was removed and 10 μl growth medium plus 100 pi CellTiter-Glo® Reagent was added to each well. After mixing and a 10 minute incubation, luminescence was recorded on an EnVision Multi-Label Reader (Perkin Elmer). Compounds in DMSO were diluted in growth medium such that the final, maximum concentration of compound was 10 μM, and serial 4-fold dilutions were performed to generate a 9-point concentration series. 10 μl of compound solution at 10 times final concentration was added to wells of the second plate. Plate was then incubated for 120 hours at 37 C and 5% CO2. On day 7 the plates were removed, 100 μl CellTiter-Glo® Reagent was added to each well, and after mixing and a 10 minute incubation, luminescence was recorded on an EnVision Multi-Label Reader (Perkin Elmer). Data was exported to GeneData Screener and modeled with a sigmoidal concentration response model in order to determine the IC50 for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite having a KRAS G12C mutation, is insensitive to the KRAS G12C (OFF) inhibitor MRTX-849 (Hallin et al., Cancer Discovery 10:54-71 (2020)), and the cell line SW1573, which is insensitive to the KRAS G12C (OFF) inhibitor AMG510 (Canon et al., Nature 575:217-223 (2019)).

TABLE 19
IC50 values for various cancer cell lines with Compound B, Compound C, and Compound D

| | | | Cmpd B | Cmpd C | Cmpd D |
|---|---|---|---|---|---|
| Cell Line | Histotype | Mutant | IC50* | IC50* | IC50* |
| A-375 | Skin | BRAF V600E | low | low | low |
| | | | sensitivity | sensitivity | sensitivity |
| KYSE- | HN/Esophagus | KRAS G12C | moderately | | very |
| 410 | | | sensitive | | sensitive |
| MIA | Pancreas | KRAS G12C | moderately | very | very |
| PaCa-2 | | | sensitive | sensitive | sensitive |
| NCI- | Lung | KRAS G12C | moderately | very | very |
| H358 | | | sensitive | sensitive | sensitive |
| SW1573 | Lung | KRAS G12C | low | low | low |
| | | | sensitivity | sensitivity | sensitivity |
| SW837 | Intestine/Large/Colorectum | KRAS G12C | moderately | | moderately |
| | | | sensitive | | sensitive |
| LS513 | Intestine/Large/Colorectum | KRAS G12D | moderately | | moderately |
| | | | sensitive | | sensitive |
| HuCCT1 | Liver/Bile duct | KRAS G12D | moderately | | very |
| | | | sensitive | | sensitive |
| HCC1588 | Lung | KRAS G12D | low | low | moderately |
| | | | sensitivity | sensitivity | sensitive |
| HPAC | Pancreas | KRAS G12D | moderately | | very |
| | | | sensitive | | sensitive |
| AsPC-1 | Pancreas | KRAS G12D | moderately | moderately | moderately |
| | | | sensitive | sensitive | sensitive |
| AGS | Stomach | KRAS G12D | moderately | very | moderately |
| | | | sensitive | sensitive | sensitive |
| HEC-1-A | Uterus | KRAS G12D | moderately | | moderately |
| | | | sensitive | | sensitive |
| SW403 | Intestine/Large/Colorectum | KRAS G12V | moderately | | very |
| | | | sensitive | | sensitive |
| NOZ | Liver/Bile duct | KRAS G12V | moderately | | moderately |
| | | | sensitive | | sensitive |
| NCI- | Lung | KRAS G12V | moderately | moderately | moderately |
| H441 | | | sensitive | sensitive | sensitive |
| NCI- | Lung | KRAS G12V | moderately | very | very |
| H727 | | | sensitive | sensitive | sensitive |
| OVCAR-5 | Ovary | KRAS G12V | moderately | | very |
| | | | sensitive | | sensitive |
| Capan-2 | Pancreas | KRAS G12V | moderately | | very |
| | | | sensitive | | sensitive |
| SW48 | Intestine/Large/Colorectum | not MAPK (PIK3CA | low | low | low |
| | | G914R, EGFR | sensitivity | sensitivity | sensitivity |
| | | G719S) | | | |
| NCI- | Lung | other KRAS (G12A) | moderately | | moderately |
| H2009 | | | sensitive | | sensitive |
| CAL-62 | HN/Thyroid | other KRAS (G12R) | moderately | | |
| | | | sensitive | | |
| A549 | Lung | other KRAS (G12S) | moderately | moderately | moderately |
| | | | sensitive | sensitive | sensitive |
| TOV-21G | Ovary | other KRAS (G13C) | low | | moderately |
| | | | sensitivity | | sensitive |
| DV-90 | Lung | other KRAS (G13D) | low | | moderately |
| | | | sensitivity | | sensitive |
| HCT116 | Intestine/Large/Colorectum | other KRAS (G13D) | moderately | | very |
| | | | sensitive | | sensitive |
| NCI- | Intestine/Large/Colorectum | other KRAS (G13D) | moderately | | very |
| H747 | | | sensitive | | sensitive |
| NCI- | Lung | other KRAS (Q61H) | moderately | moderately | moderately |
| H460 | | | sensitive | sensitive | sensitive |
| Calu-6 | Lung | other KRAS (Q61K) | moderately | very | moderately |
| | | | sensitive | sensitive | sensitive |
| SNU-668 | Stomach | other KRAS (Q61K) | moderately | | very |
| | | | sensitive | | sensitive |
| OZ | Liver/Bile duct | other KRAS (Q61L) | moderately | | moderately |
| | | | sensitive | | sensitive |
| SW948 | Intestine/Large/Colorectum | other KRAS (Q61L) | low | moderately | moderately |
| | | | sensitivity | sensitive | sensitive |
| BxPC-3 | Pancreas | other MAPK (BRAF | low | low | low |
| | | V487_P492delinsA) | sensitivity | sensitivity | sensitivity |
| NCI- | Lung | other MAPK (EGFR | moderately | moderately | very |
| H1975 | | T790M, L858R) | sensitive | sensitive | sensitive |
| NCI- | Lung | other MAPK | moderately | | moderately |
| H3122 | | (EML4- | sensitive | | sensitive |
| | | ALK(E13, A20)) | | | |
| YCC-1 | Stomach | other MAPK (KRAS | | | moderately |
| | | Amp) | | | sensitive |
| MeWo | Skin | other MAPK (NF1 | low | moderately | moderately |
| | | mut) | sensitivity | sensitive | sensitive |
| NCI- | Lung | other MAPK (NF1 | moderately | moderately | moderately |
| H1838 | | mut) | sensitive | sensitive | sensitive |
| RL95-2 | Uterus | other RAS (HRAS | | | very |
| | | Q61H) | | | sensitive |
| NCI- | Lung | other RAS (HRAS | | moderately | moderately |
| H1915 | | Q61L) | | sensitive | sensitive |
| L-363 | Blood/Leukemia | other RAS (NRAS | | | low |
| | | Q61H) | | | sensitivity |
| CHP-212 | Brain&Nerves | other RAS (NRAS | | | moderately |
| | | Q61K) | | | sensitive |
| HT-1080 | Soft tissue | other RAS (NRAS | | | moderately |
| | | Q61K) | | | sensitive |
| NCI- | Lung | other RAS (NRAS | | | very |
| H2087 | | Q61K) | | | sensitive |
| OCI-LY- | Blood/Lymphoma | other RAS (NRAS | | | moderately |
| 19 | | Q61K) | | | sensitive |
| SNU-387 | Liver/bile duct | other RAS (NRAS | | | moderately |
| | | Q61K) | | | sensitive |
| Hep G2 | Liver/bile duct | other RAS (NRAS | | moderately | very |
| | | Q61L) | | sensitive | sensitive |
| HL-60 | Blood/Leukemia | other RAS (NRAS | | | very |
| | | Q61L) | | | sensitive |
| MOLP8 | Blood/Myeloma | other RAS (NRAS | | | moderately |
| | | Q61L) | | | sensitive |
| SNU-719 | Stomach | other RAS (NRAS | | | moderately |
| | | Q61L) | | | sensitive |
| TF-1 | Blood/Leukemia | other RAS (NRAS | | | moderately |
| | | Q61P) | | | sensitive |
| ASH-3 | HN/Thyroid | other RAS (NRAS | | | moderately |
| | | Q61R) | | | sensitive |
| SK-MEL- | Skin | other RAS (NRAS | | | moderately |
| 2 | | Q61R) | | | sensitive |
| SW1271 | Lung | other RAS (NRAS | | | moderately |
| | | Q61R) | | | sensitive |

*Key:
low sensitivity: IC50 ≥ 1 uM
moderately sensitive: 1 uM > IC50 ≥ 0.1 uM
very sensitive: IC50 < 0.1 uM

In Vivo PD and Efficacy Data with Compound A, a Compound of the Present Invention

### FIG. 1A:

Methods: The human pancreatic adenocarcinoma Capan-2 KRASG12V/wt xenograft model was used for a single-day treatment PK/PD study (FIG. A). Compound A (Capan-2 pERK K-Ras G12D EC50: 0.0037 uM) was administered at 100 mg/kg as a single dose or bid (second dose administered 8 hours after first dose) orally administered (po). The treatment groups with sample collections at various time points were summarized in Table 20 below. Tumor samples were collected to assess RAS/ERK signaling pathway modulation by measuring the mRNA level of human DUSP6 in qPCR assay, while accompanying blood plasma samples were collected to measure circulating Compound A levels.

TABLE 20
Summary of treatment groups, doses, and time points
for single-dose PK/PD study using Capan-2 tumors.

| | | PK, n = 3/time | PD, n = 3/time |
|---|---|---|---|
| Compound/group | Dose/Regimen | point | point |
| Vehicle control | 10 ml/kg ip | 1 h, 24 h | 1 h, 24 h |
| Compound A | 100 mg/kg po | 1 h, 2 h, 8 h, | 1 h, 2 h, 8 h, |
| | | 12 h, 24 h | 10 h, 24 h |
| Compound A | 100 mg/kg po | 1 h, 2 h, 8 h, | 1 h, 2 h, 8 h, |
| | bid | 12 h, 24 h | 10 h, 24 h |

Results: In FIG. 1A, Compound A delivered at 100 mg/kg as a single dose inhibited DUSP6 mRNA levels in tumors >95% through 10 hours. A second dose of Compound A 8 hours following first administration maintained pathway modulation of 93% through 24 hours. These data indicate Compound A provides strong MAPK pathway modulation with continued target coverage.

### FIG. 1B:

Methods: Effects of Compound A on tumor cell growth in vivo were evaluated in the human pancreatic adenocarcinoma Capan-2 KRASG12V/wt xenograft model using female BALB/c nude mice (6-8 weeks old). Mice were implanted with Capan-2 tumor cells in 50% Matrigel (4×106 cells/mouse) subcutaneously in the flank. Once tumors reached an average size of ˜180 mm3, mice were randomized to treatment groups to start the administration of test articles or vehicle. Compound A was orally administered (po) twice daily at 100 mg/kg. A SHP2 inhibitor, RMC-4550 (commercially available), was administered orally every other day at 20 mg/kg. Body weight and tumor volume (using calipers) was measured twice weekly until study endpoints. Tumor regressions calculated as >10% decrease in starting tumor volume. All dosing arms were well tolerated.

Results: In FIG. 1B, single agent SHP2i RMC-4550 dosed every other day at 20 mg/kg po resulted in 39% TGI. Single-agent Compound A administered at 100 mg/kg po bid daily led to a TGI of 98%, with 4/10 (40%) individual animals achieving tumor regressions. Combination of Compound A and RMC-4550 resulted in total tumor regression of 35%, with individual tumor regressions observed in 7/9 (77.8%) individual animals at the end of treatment (Day 40 after treatment started) in Capan-2 CDX model with heterozygous KRASG12V. The anti-tumor activity of Compound A, and Combination arms was statistically significant compared with control group (***p<0.001, ordinary One-way ANOVA with multiple comparisons via a post-hoc Tukey's test), while RMC-4550 was not significant at these doses.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### APPENDIX D-1

### C40-, C28-, and C-32-Linked Rapamycin Analogs as mTOR Inhibitors

### Field of the Disclosure

The present disclosure relates to mTOR inhibitors. Specifically, the embodiments are directed to compounds and compositions inhibiting mTOR, methods of treating diseases mediated by mTOR, and methods of synthesizing these compounds.

### Background of the Disclosure

The mammalian target of rapamycin (mTOR) is a serine-threonine kinase related to the lipid kinases of the phosphoinositide 3-kinase (PI3K) family, mTOR exists in two complexes, mTORC1 and mTORC2, which are differentially regulated, have distinct substrate specificities, and are differentially sensitive to rapamycin, mTORC1 integrates signals from growth factor receptors with cellular nutritional status and controls the level of cap-dependent mRNA translation by modulating the activity of key translational components such as the cap-binding protein and oncogene eIF4E.

mTOR signaling has been deciphered in increasing detail. The differing pharmacology of inhibitors of mTOR has been particularly informative. The first reported inhibitor of mTOR, Rapamycin is now understood to be an incomplete inhibitor of mTORC1. Rapamycin is a selective mTORC1 inhibitor through the binding to the FK506 Rapamycin Binding (FRB) domain of mTOR kinase with the aid of FK506 binding protein 12 (FKBP12). The FRB domain of mTOR is accessible in the mTORC1 complex, but less so in the mTORC2 complex. Interestingly, the potency of inhibitory activities against downstream substrates of mTORC1 by the treatment of Rapamycin is known to be diverse among the mTORC1 substrates. For example. Rapamycin strongly inhibits phosphorylation of the mTORC1 substrate S6K and, indirectly, phosphorylation of the downstream ribosomal protein S6 which control ribosomal biogenesis. On the other hand, Rapamycin shows only partial inhibitory activity against phosphorylation of 4E-BP1, a major regulator of eIF4E which controls the initiation of CAP-dependent translation. As a result, more complete inhibitors of mTORC1 signaling are of interest.

A second class of “ATP-site” inhibitors of mTOR kinase were reported. This class of mTOR inhibitors will be referred to as TORi (ATP site TOR inhibitor). The molecules compete with ATP, the substrate for the kinase reaction, in the active site of the mTOR kinase (and are therefore also mTOR active site inhibitors). As a result, these molecules inhibit downstream phosphorylation of a broader range of substrates.

Although mTOR inhibition may have the effect of blocking 4E-BP1 phosphorylation, these agents may also inhibit mTORC2, which leads to a block of Akt activation due to inhibition of phosphorylation of Akt S473.

Disclosed herein, inter alia, are mTOR inhibitors. In some embodiments, compounds disclosed herein are more selective inhibitors of mTORC1 versus mTORC2. In some embodiments, compounds disclosed herein are more selective inhibitors of mTORC2 versus mTORC1. In some embodiments, compounds disclosed herein exhibit no selectivity difference between mTORC1 and mTORC2.

### SUMMARY OF THE DISCLOSURE

The present disclosure relates to compounds capable of inhibiting the activity of mTOR. The present disclosure further provides a process for the preparation of compounds of the present disclosure, pharmaceutical preparations comprising such compounds and methods of using such compounds and compositions in the management of diseases or disorders mediated by mTOR.

The present disclosure provides a compound of Formula Ic:

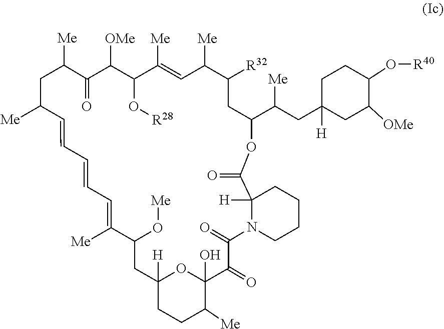

or a pharmaceutically acceptable salt or tautomer thereof, wherein:

R³² is —H, ═O, —OR³, —N₃, or —O—C(═Z¹)—R^{32a};

R²⁸ is —H, (C₁-C₆)alkyl, or —C(═Z¹)—R^{28a};

R⁴⁰ is —H or —C(═Z¹)—R^{40a};
wherein when R²⁸ and R⁴⁰ are H, then R³² is not ═O;

each Z¹ is independently O or S;

R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;

A¹ and A² are independently absent or are independently selected from

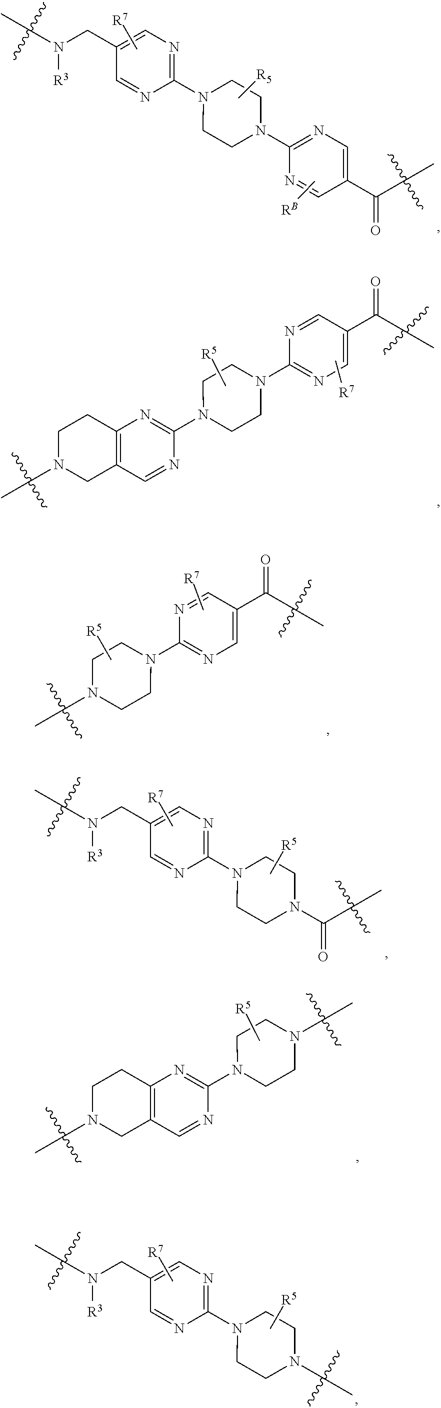

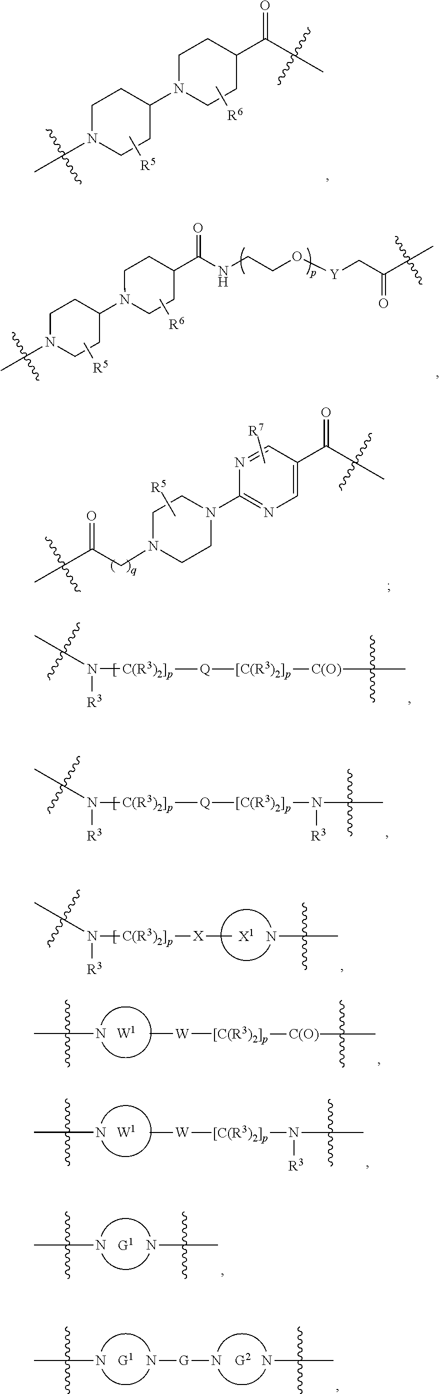

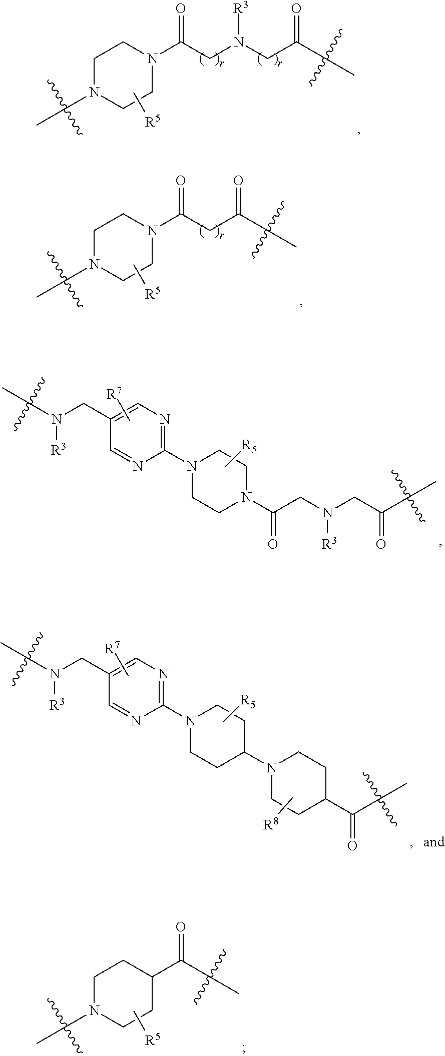

wherein the bond on the left side of A¹, as drawn, is bound to —C(═Z¹)— or L²; and wherein the bond on the right side of the A² moiety, as drawn, is bound to B or L³;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring; each G is independently absent or a ring selected from arylene, cycloalkylene, heteroarylene, and heterocyclylene;

each G¹ and G² are independently heteroarylene or heterocyclylene ring;

each L¹ is independently selected from

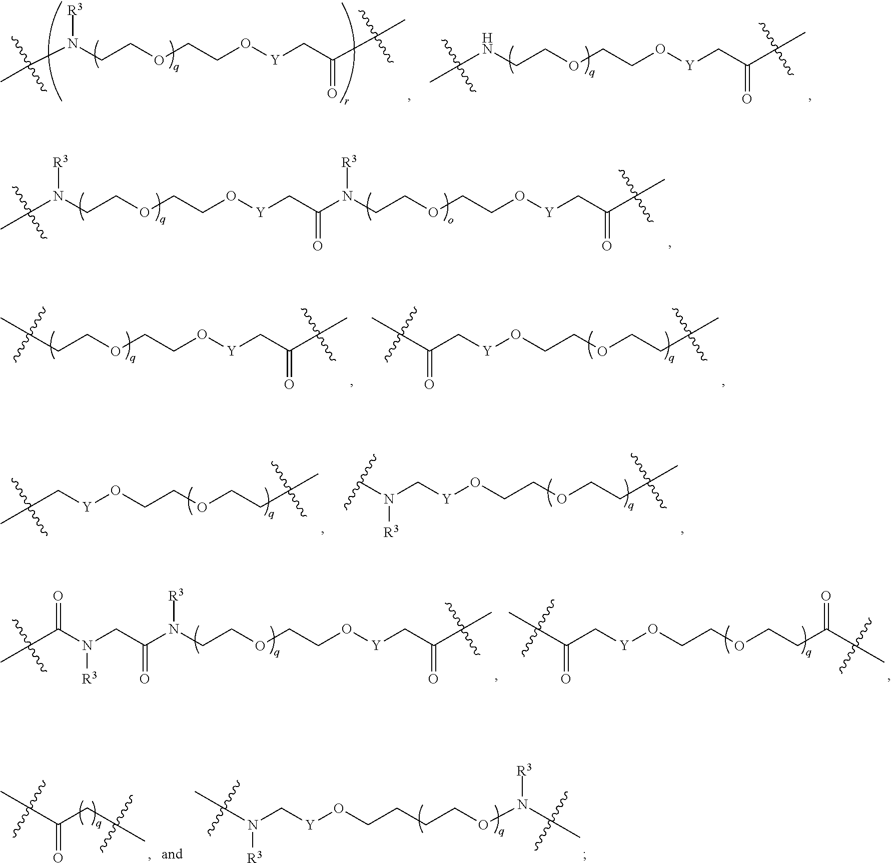

L² and L³ are independently absent or are independently selected from

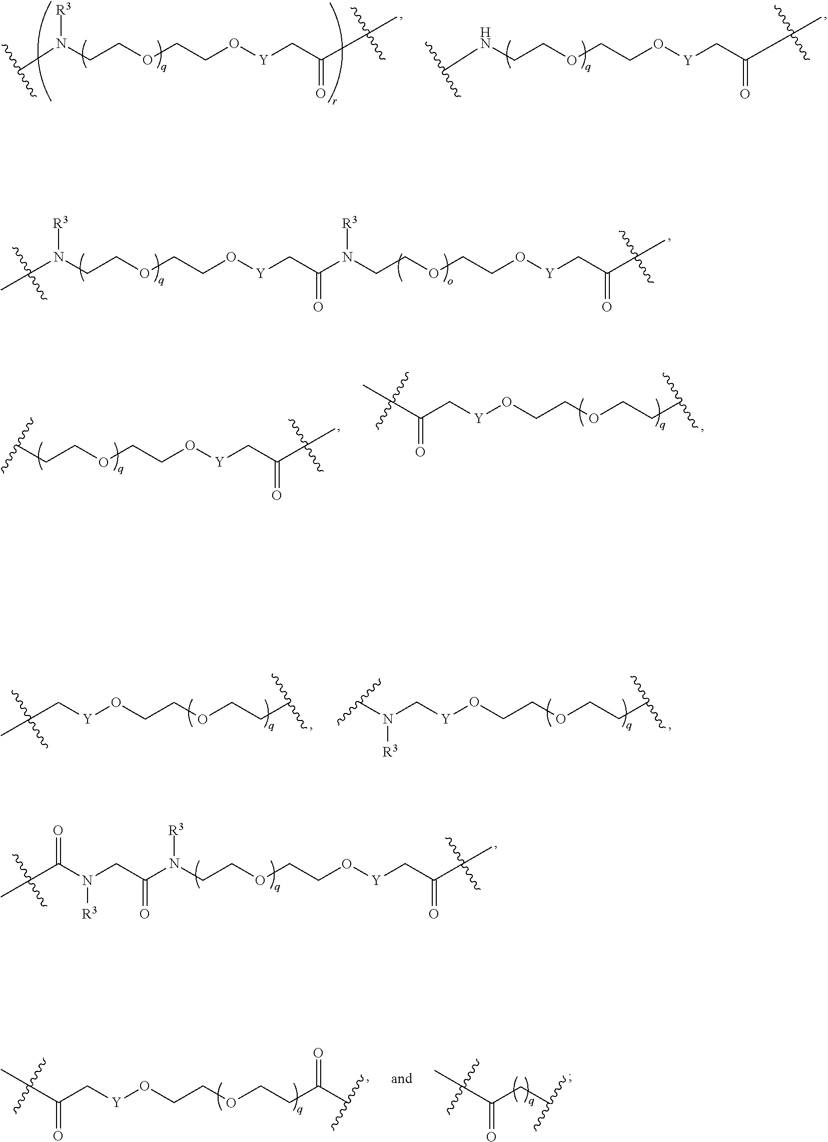

each B is independently selected from

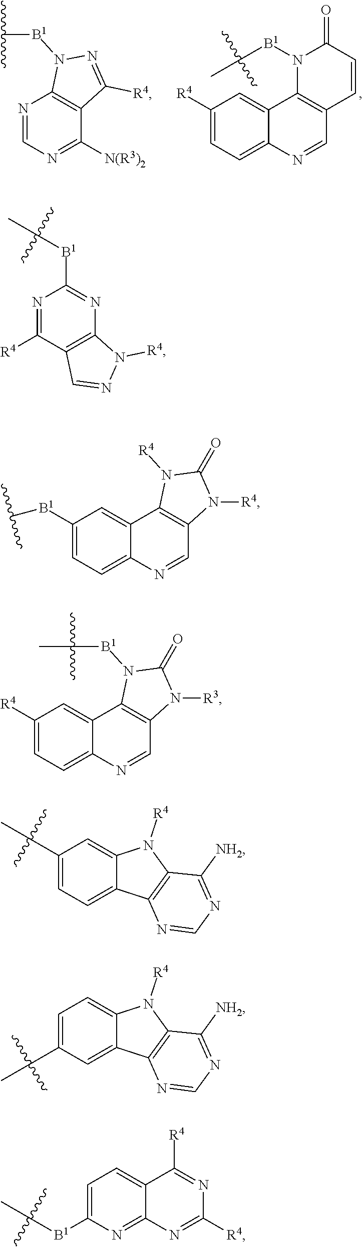

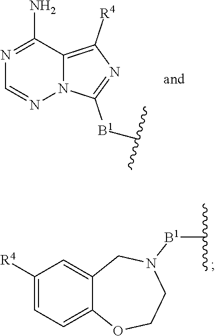

each B¹ is independently selected from

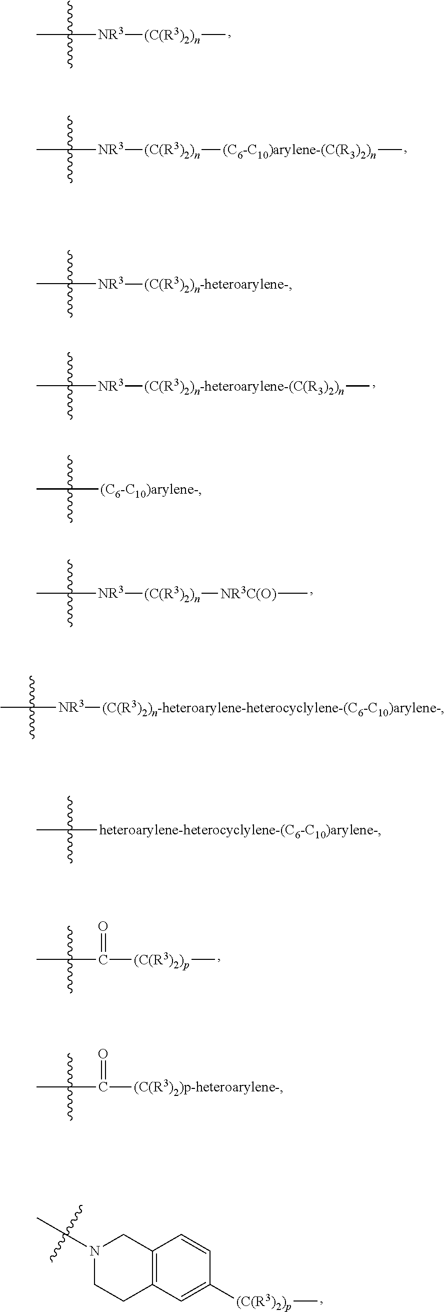

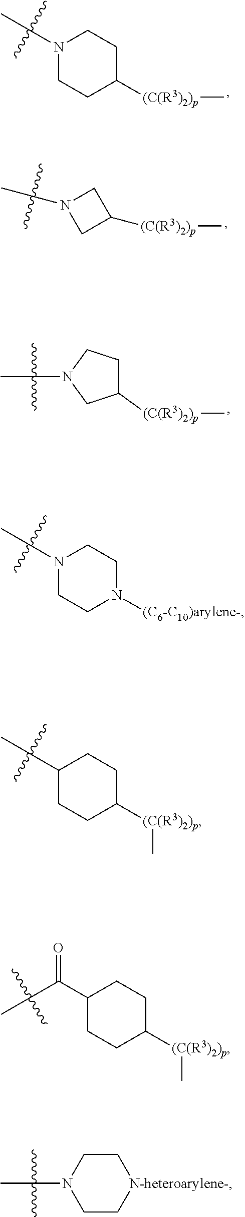

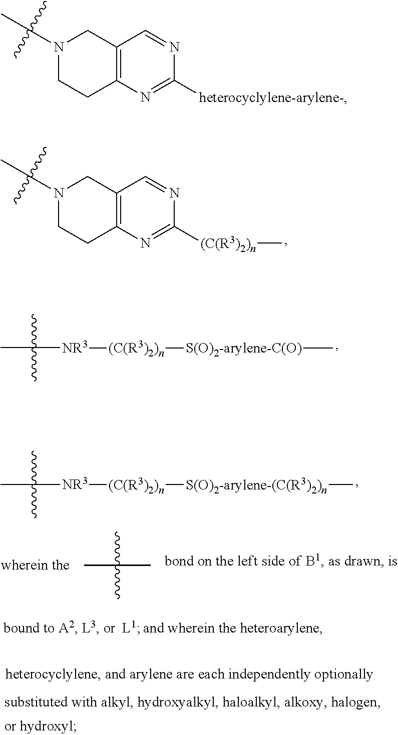

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each R⁵ is independently H, (C₁-C₆)alkyl, —C(O)OR³, or —N(R³)₂, wherein the alkyl of (C₁-C₆)alkyl is each independently optionally substituted with —N(R³)₂ or —OR³;

each R⁶ is independently H, (C₁-C₆)alkyl, —C(O)OR³, or —N(R³)₂, wherein the alkyl of (C₁-C₆)alkyl is each independently optionally substituted with —N(R³)₂ or —OR³;

each R⁷ is independently H, (C₁-C₆)alkyl, —C(O)OR³, or —N(R³)₂, wherein the alkyl of (C₁-C₆)alkyl is each independently optionally substituted with —N(R³)₂ or —OR³;

each R⁸ is independently H, (C₁-C₆)alkyl, —C(O)OR³, or —N(R³)₂, wherein the alkyl of (C₁-C₆)alkyl is each independently optionally substituted with —N(R³)₂ or —OR³;

each Y is independently —C(R³)₂ or a bond;

each n is independently an integer from one to 12;

each o is independently an integer from zero to 30;

each p is independently an integer from zero to 12;

each q is independently an integer from zero to 30; and

each r is independently an integer from one to 6.

The present disclosure provides a compound of Formula Ia:

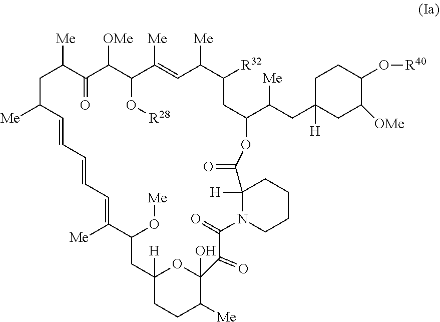

or a pharmaceutically acceptable salt or tautomer thereof, wherein:

R³² is —H, ═O, —OR³, —N₃, or —O—C(═Z¹)—R^{32a};

R²⁸ is —H, (C₁-C₆)alkyl, or —C(═Z¹)—R^{28a};

R⁴⁰ is —H or —C(═Z¹)—R^{40a};
wherein when R²⁸ and R⁴⁰ are H, then R³² is not ═O;

each Z¹ is independently O or S;

R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;

A¹ and A² are independently absent or are independently selected from

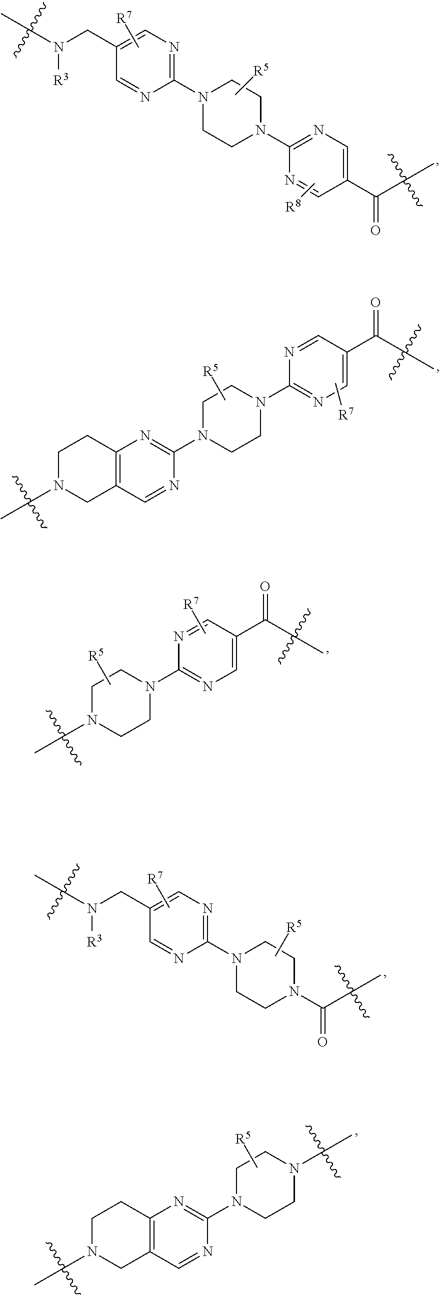

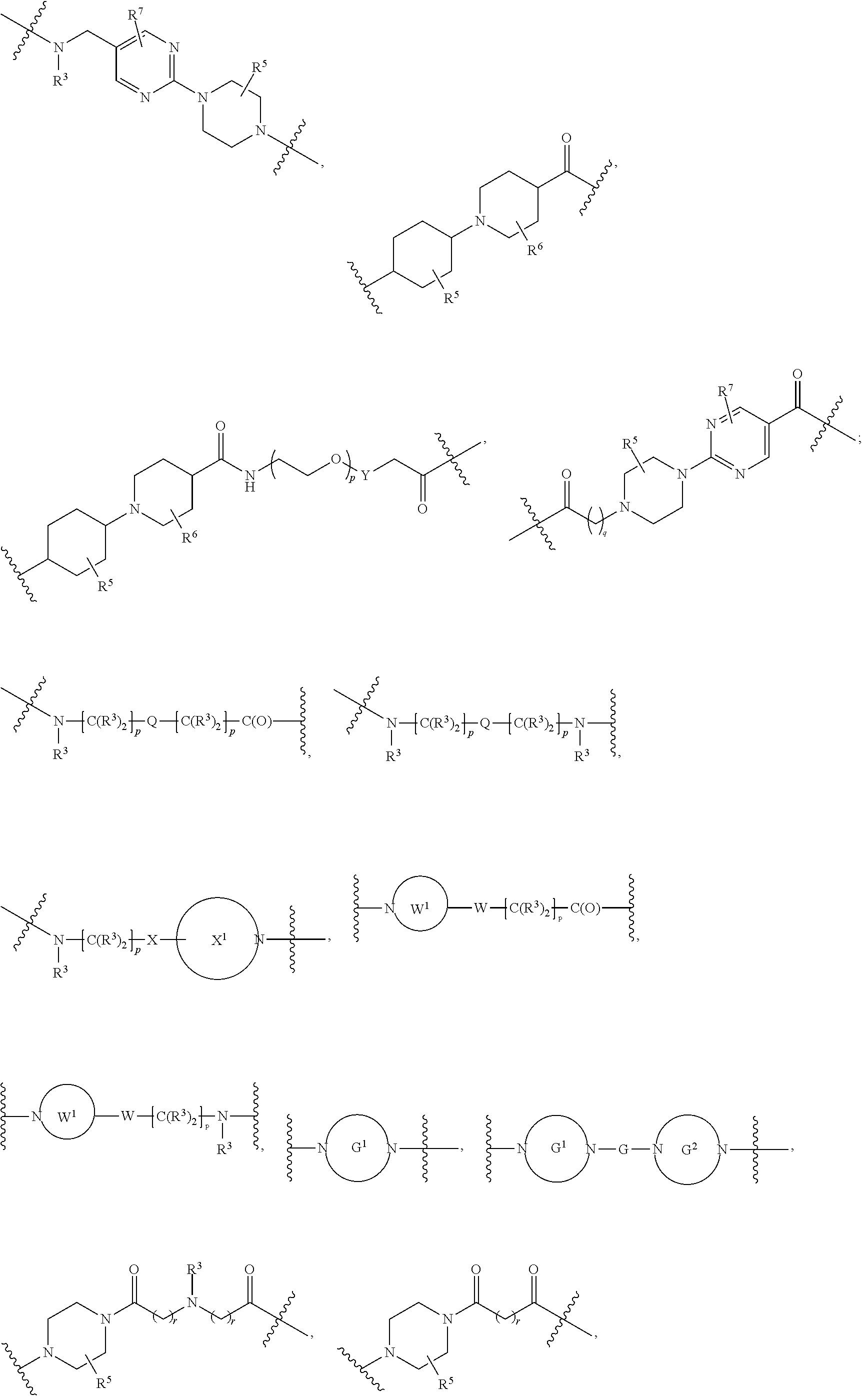

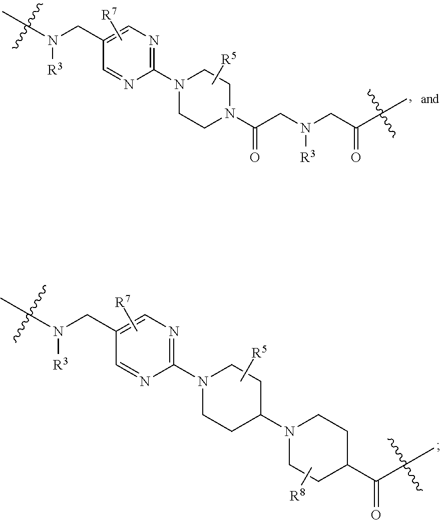

wherein the bond on the left side of A¹, as drawn, is bound to —C(═Z¹)— or L²; and wherein the bond on the right side of the A² moiety, as drawn, is bound to B or L³;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ independently is a heteroarylene or heterocyclylene ring;

each W is independently absent or 1 to 2 rings selected from arylene, cycloalkylene, heteroarylene, and heterocyclylene;

each W¹ independently is a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

each L¹ is independently selected from

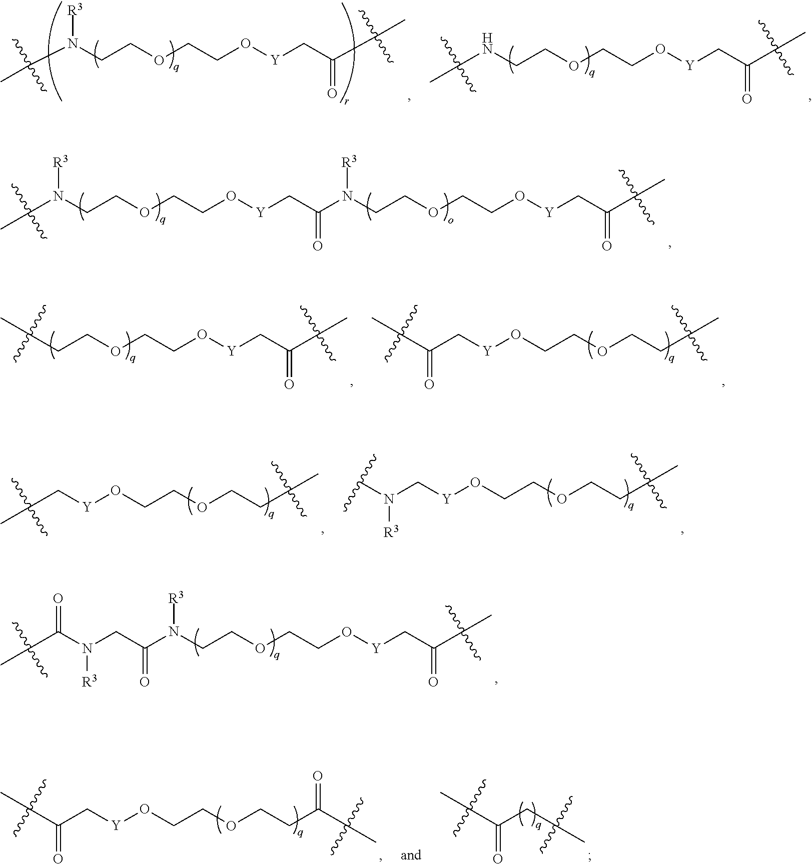

L² and L³ are independently absent or are independently selected from

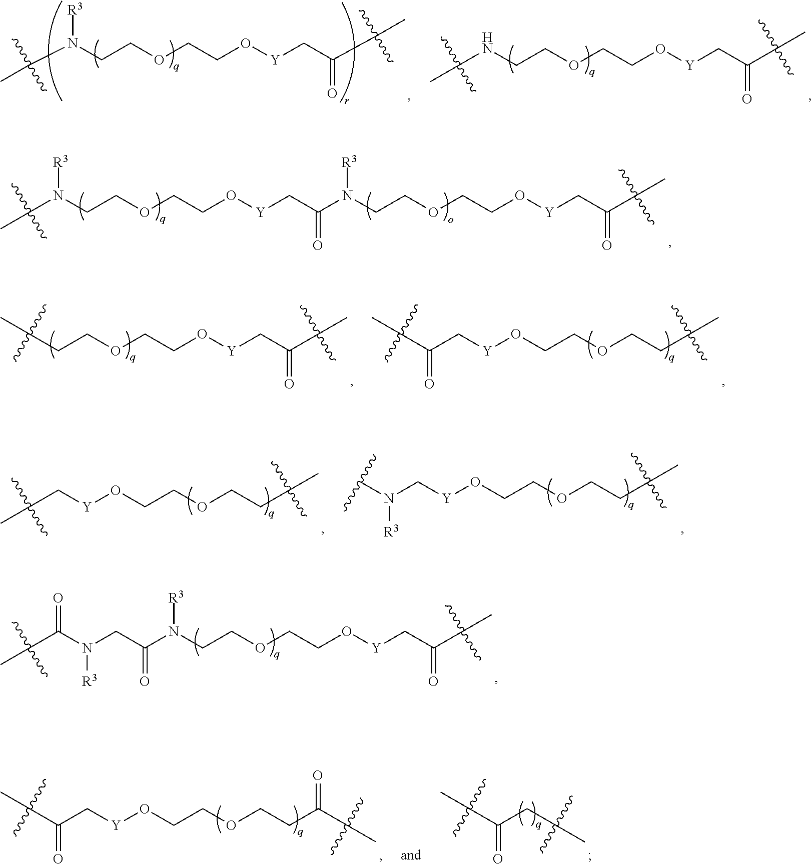

each B is independently selected from

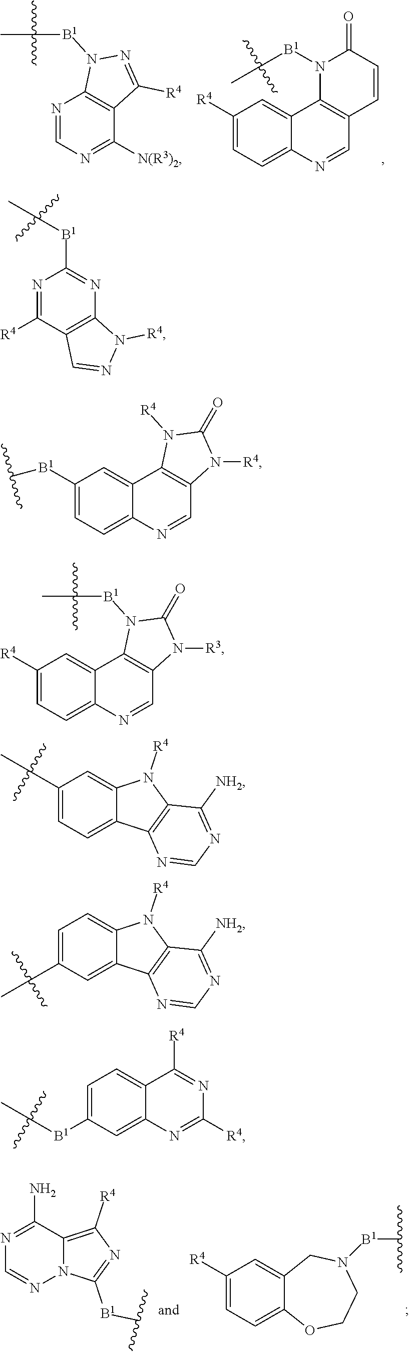

each B¹ is independently selected from

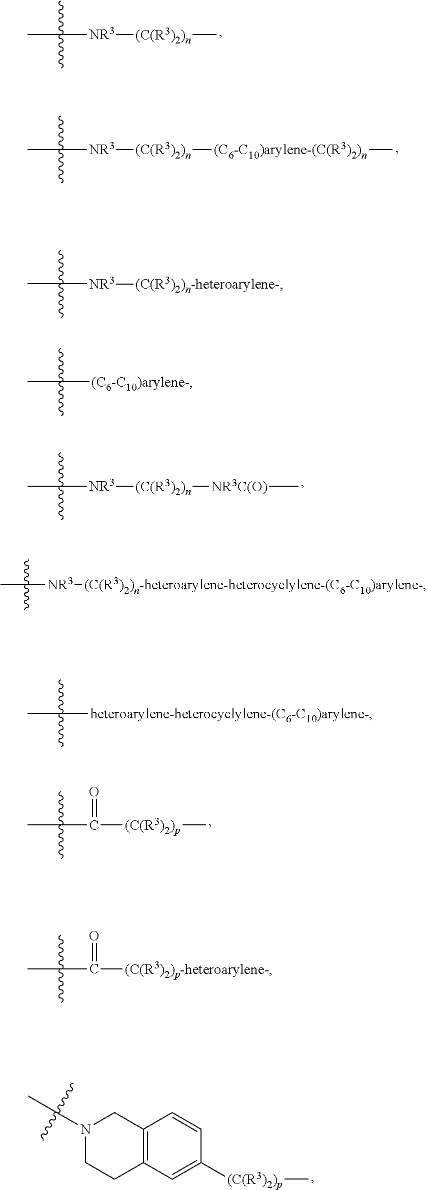

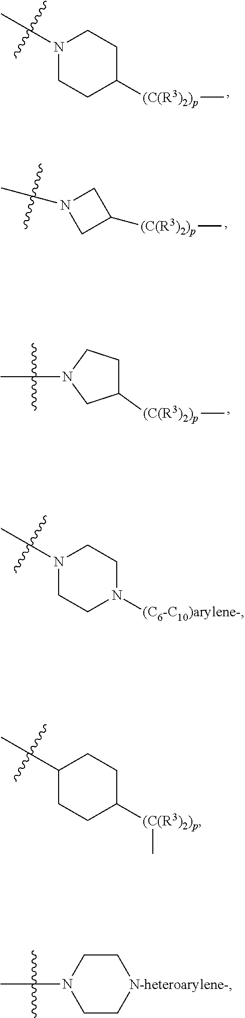

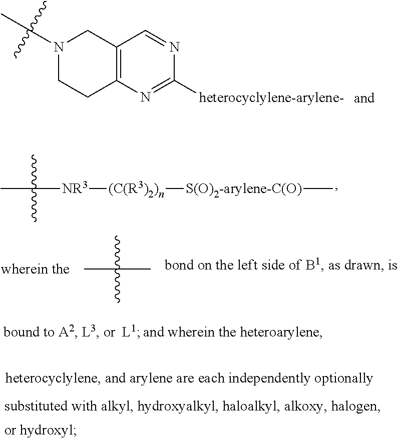

each R³ is independently H or (C₁-C₆)alkyl;

each R⁴ is independently H, (C₁-C₆)alkyl, halogen, 5-12 membered heteroaryl, 5-12 membered heterocyclyl. (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each R⁵ is independently H, (C₁-C₆)alkyl, —C(O)OR³, or —N(R³)₂, wherein the alkyl of (C₁-C₆)alkyl is each independently optionally substituted with —N(R³)₂ or —OR³;

each R⁶ is independently H, (C₁-C₆)alkyl, —C(O)OR³, or —N(R³)₂, wherein the alkyl of (C₁-C₆)alkyl is each independently optionally substituted with —N(R³)₂ or —OR³;

each R⁷ is independently H, (C₁-C₆)alkyl, —C(O)OR³, or —N(R³)₂, wherein the alkyl of (C₁-C₆)alkyl is each independently optionally substituted with —N(R³)₂ or —OR³;

each R⁸ is independently H, (C₁-C₆)alkyl, —C(O)OR³, or —N(R³)₂, wherein the alkyl of (C₁-C₆)alkyl is each independently optionally substituted with —N(R³)₂ or —OR³;

each Y is independently —C(R³)₂ or a bond;

each n is independently an integer from one to 12;

each o is independently an integer from zero to 30;

each p is independently an integer from zero to 12;

each q is independently an integer from zero to 30; and

each r is independently an integer from one to 6.

The present disclosure provides a compound of Formula I:

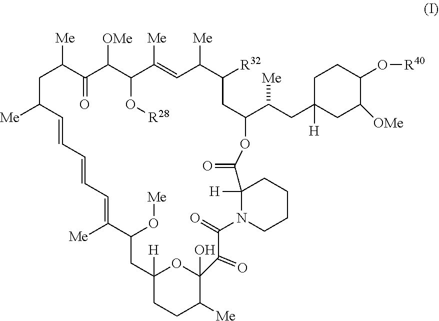

or a pharmaceutically acceptable salt or tautomer thereof, wherein:

R³² is —H, ═O, or —OR³;

R²⁸ is —H, or —C(═Z¹)—R^{28a};

R⁴⁰ is —H or —C(═Z¹)—R^{40a};
wherein at least one of R²⁸ and R⁴⁰ is not H;

each Z¹ is independently O or S;

R^{28a} and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;

A¹ and A² are independently absent or are independently selected from

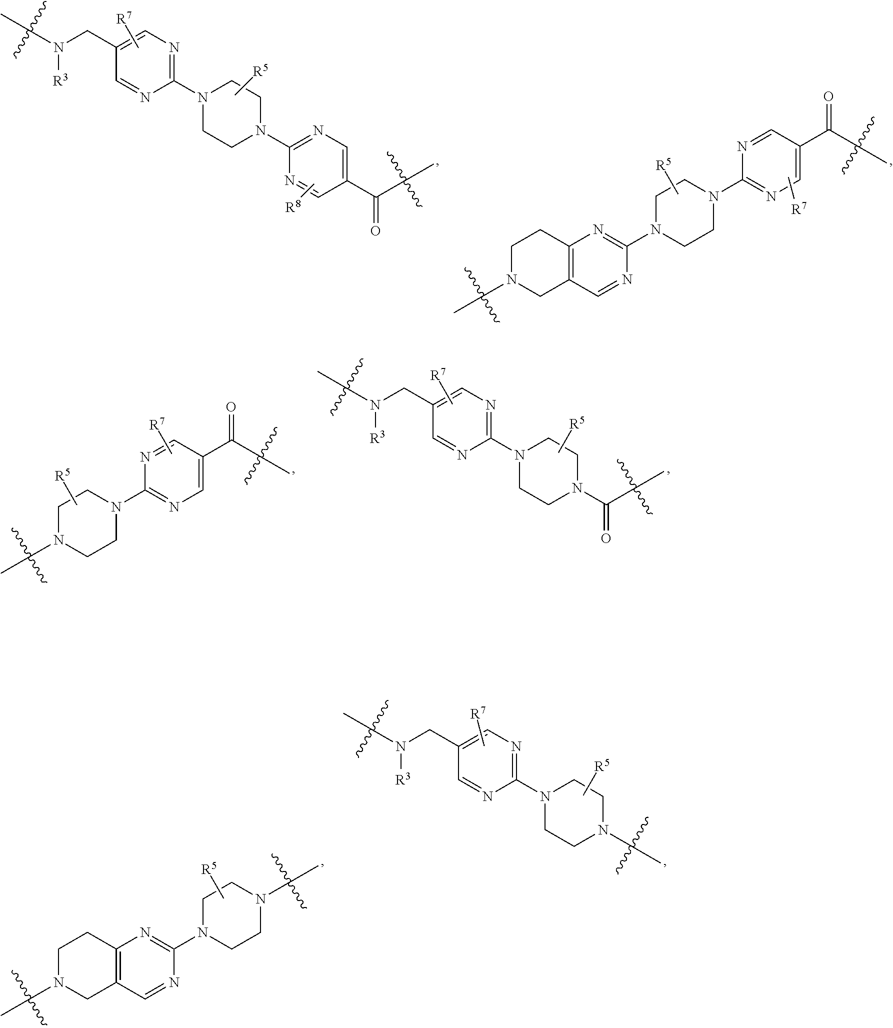

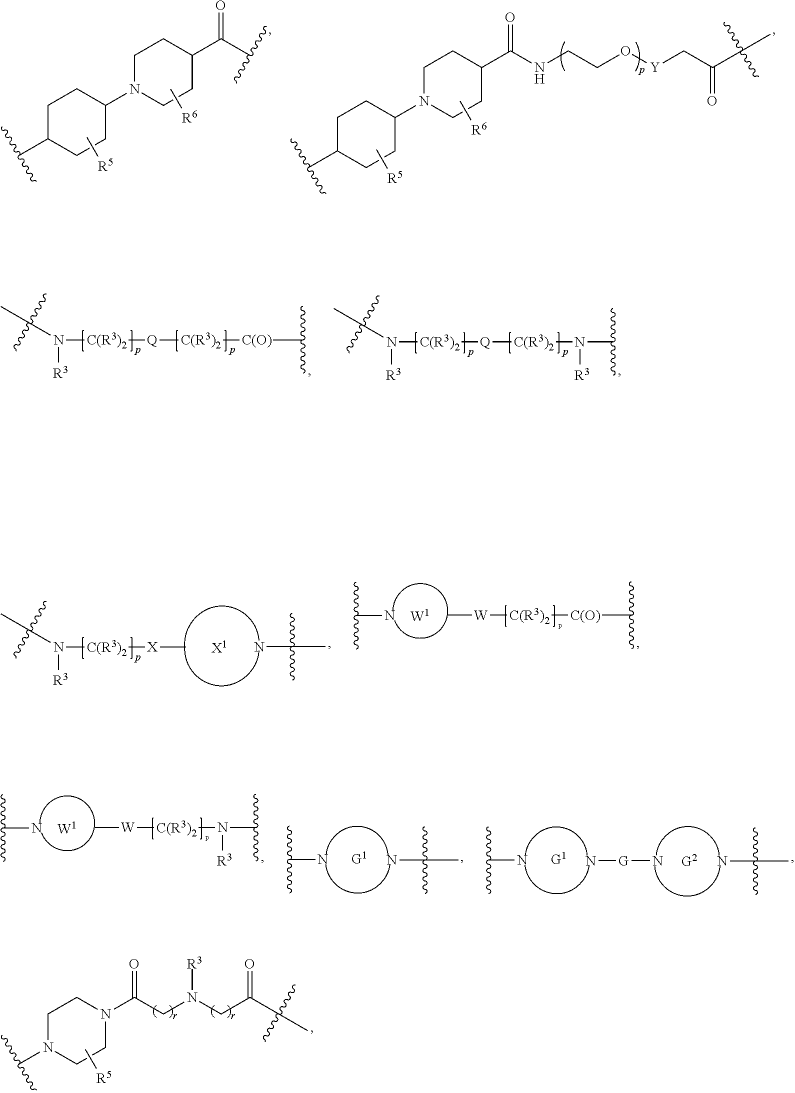

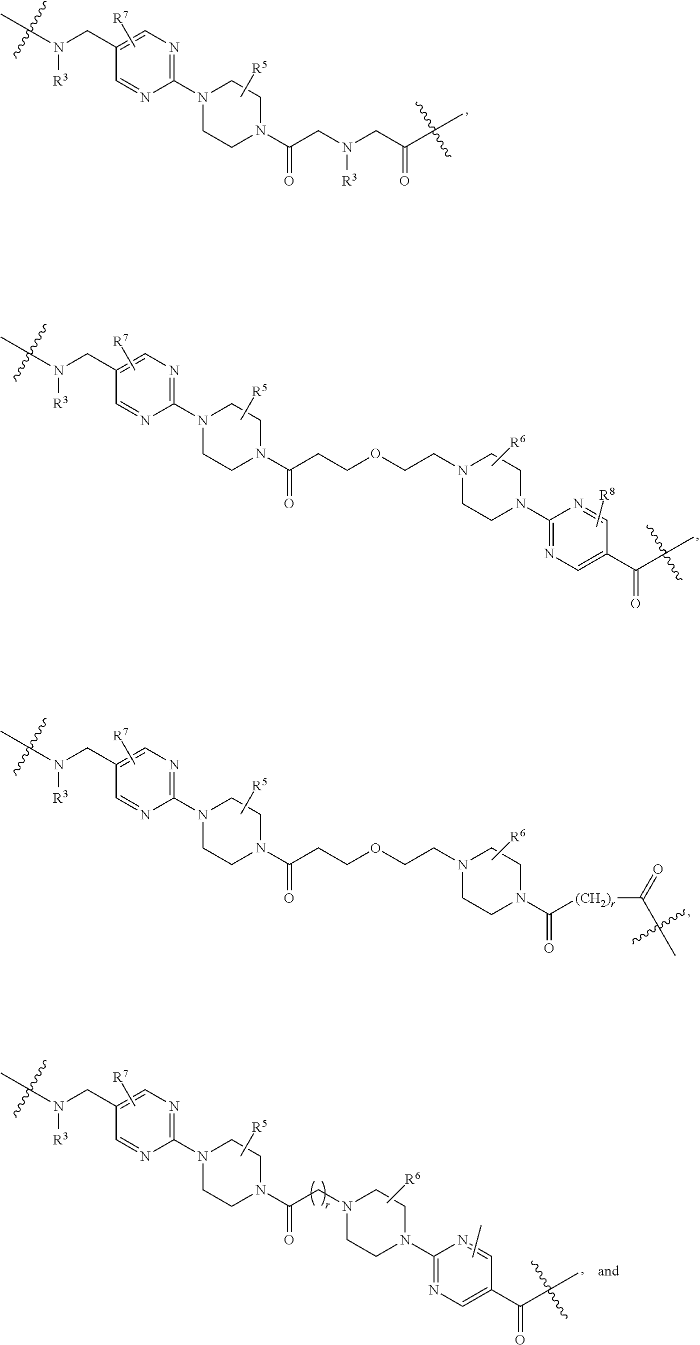

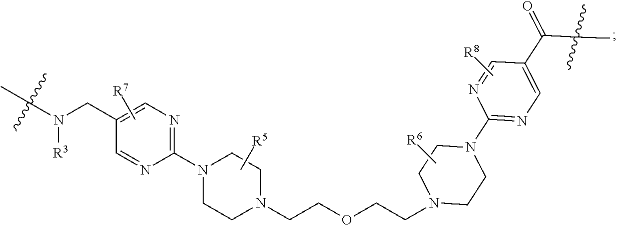

wherein the bond on the left side of A¹, as drawn, is bound to —C(═Z¹)— or L²; and wherein the bond on the right side of the A² moiety, as drawn, is bound to B or L³;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ independently is a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

each L¹ is independently selected from

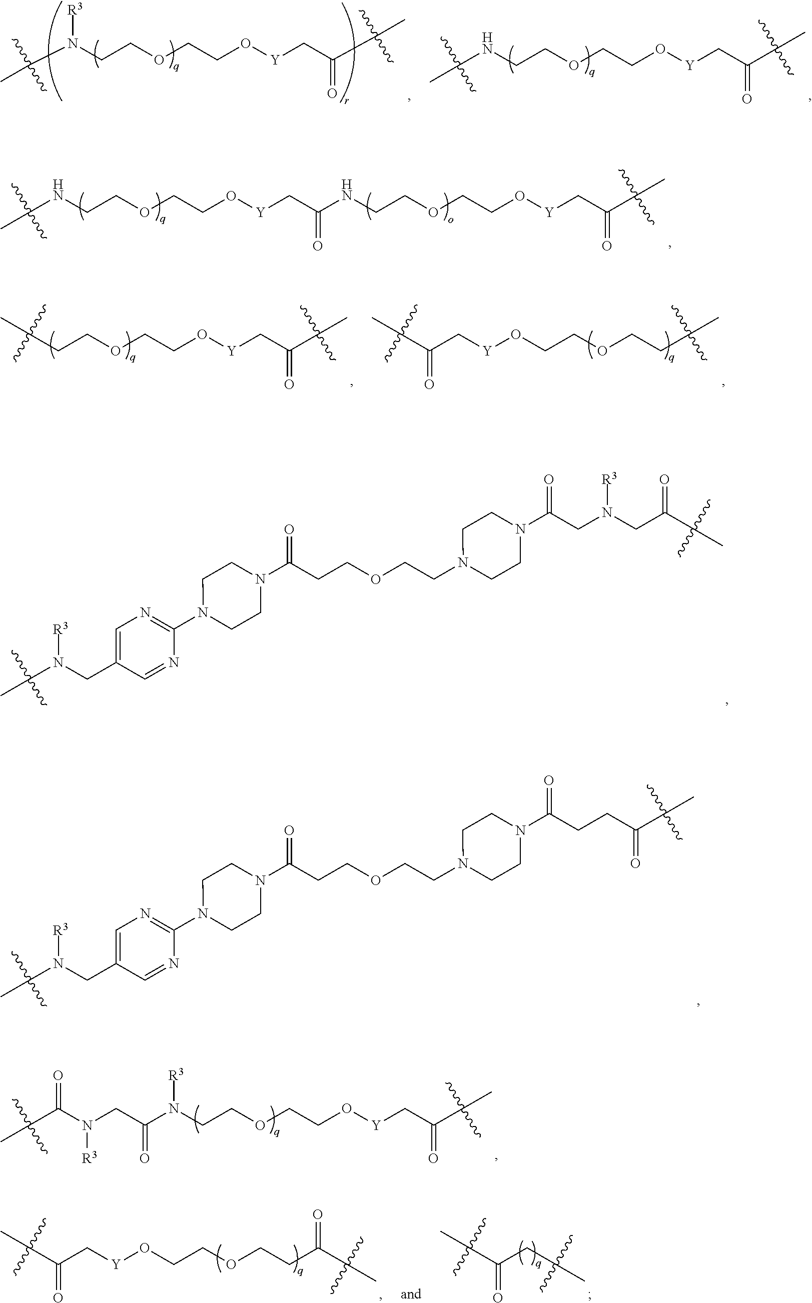

L² and L³ are independently absent or are independently selected from

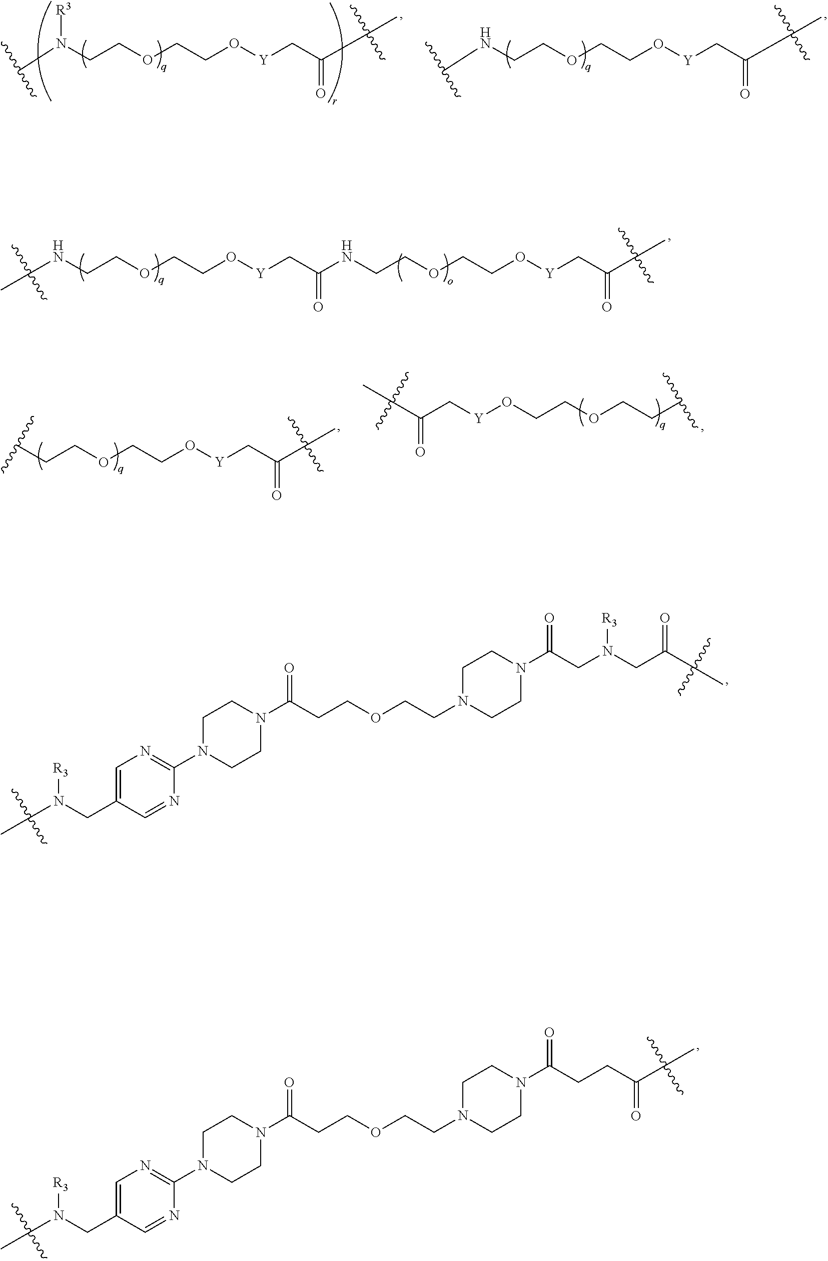

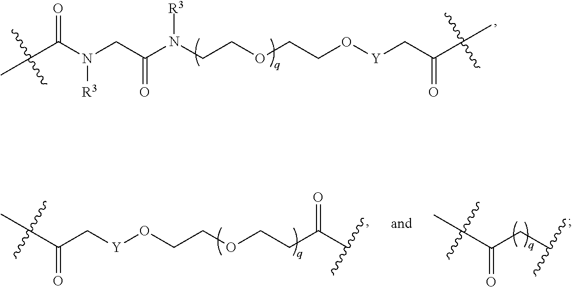

each B is independently selected from

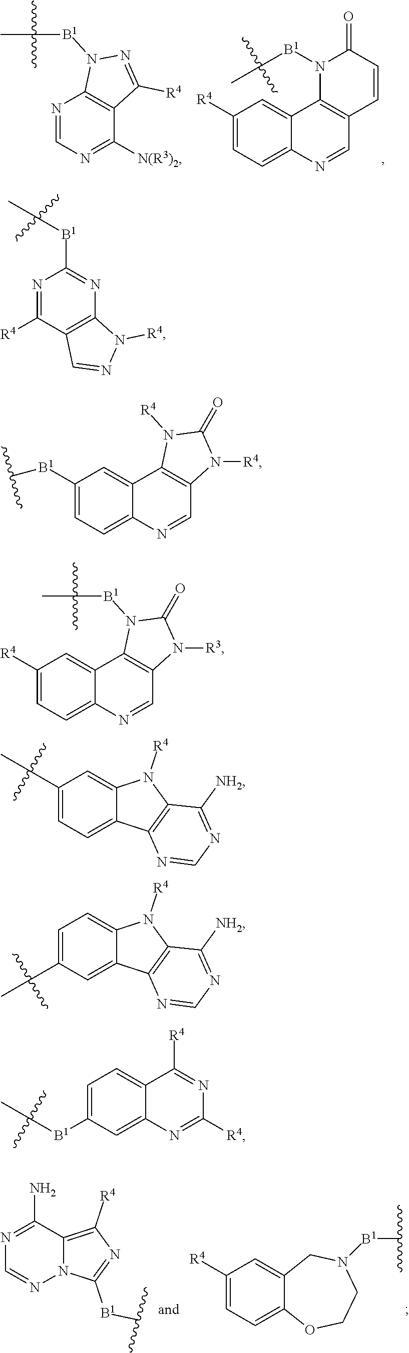

each B¹ is independently selected from

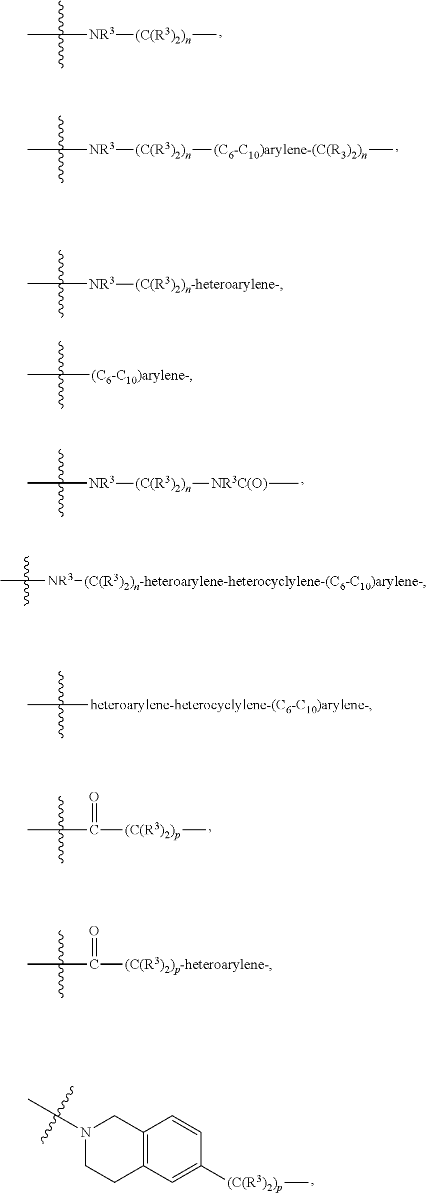

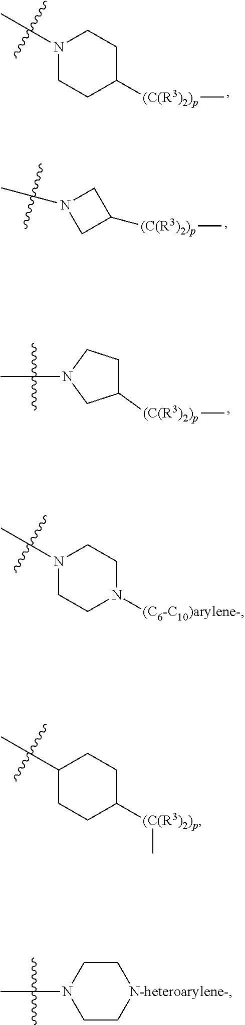

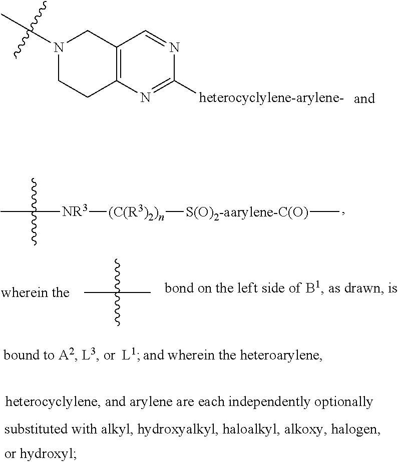

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each R⁵ is independently H, (C₁-C₆)alkyl, —C(O)OR³, or —N(R³)₂, wherein the alkyl of (C₁-C₆)alkyl is optionally substituted with —N(R³)₂ or —OR³;

each R⁶ is independently H, (C₁-C₆)alkyl, —C(O)OR³, or —N(R³)₂, wherein the alkyl is of (C₁-C₆)alkyl optionally substituted with —N(R³)₂ or —OR³;

each R⁷ is independently H, (C₁-C₆)alkyl, —C(O)OR³, or —N(R³)₂, wherein the alkyl of (C₁-C₆)alkyl is optionally substituted with —N(R³)₂ or —OR³;

each R⁸ is independently H, (C₁-C₆)alkyl, —C(O)OR³, or —N(R³)₂, wherein the alkyl of (C₁-C₆)alkyl is optionally substituted with —N(R³)₂ or —OR³;

each Y is independently —C(R³)₂ or a bond;

each n is independently an integer from one to 12;

each o is independently an integer from zero to 30;

each p is independently an integer from zero to 12;

each q is independently an integer from zero to 30; and

each r is independently an integer from one to 6.

The present disclosure provides a compound of Formula II:

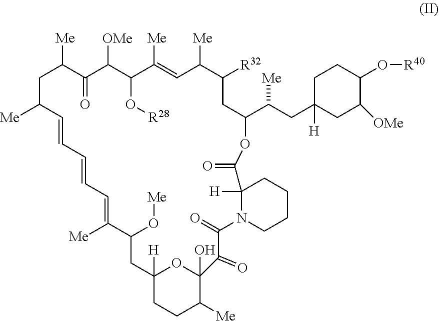

or a pharmaceutically acceptable salt or tautomer thereof, wherein:

R³² is —H, ═O or —OR³;

R²⁸ is —H or —(Z¹)—R^{28a};

R⁴⁰ is —H or —C(═Z¹)—R^{40a};
wherein at least one of R²⁸ and R⁴⁰ is not H;

Z¹ is independently O or S:

R^{28a} and R^{40a} are independently -A¹-L¹-A²-B; -A¹-A²-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;

A¹ and A² are independently absent or are independently selected from

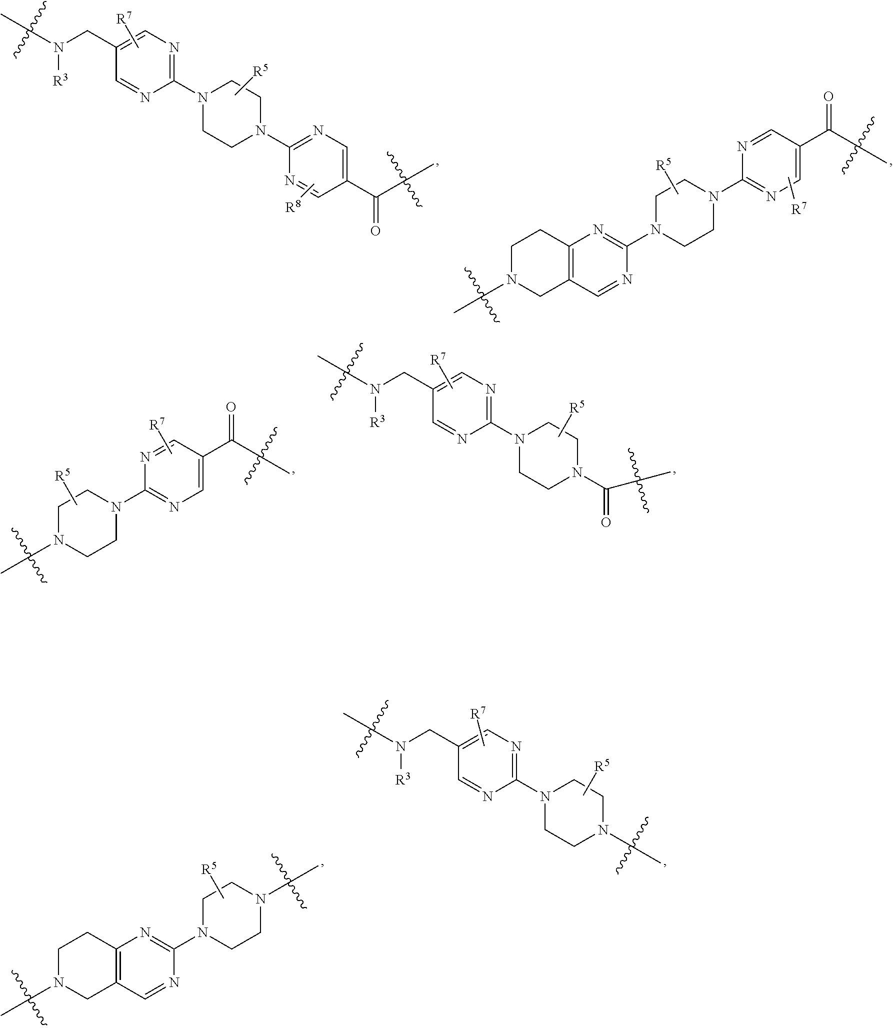

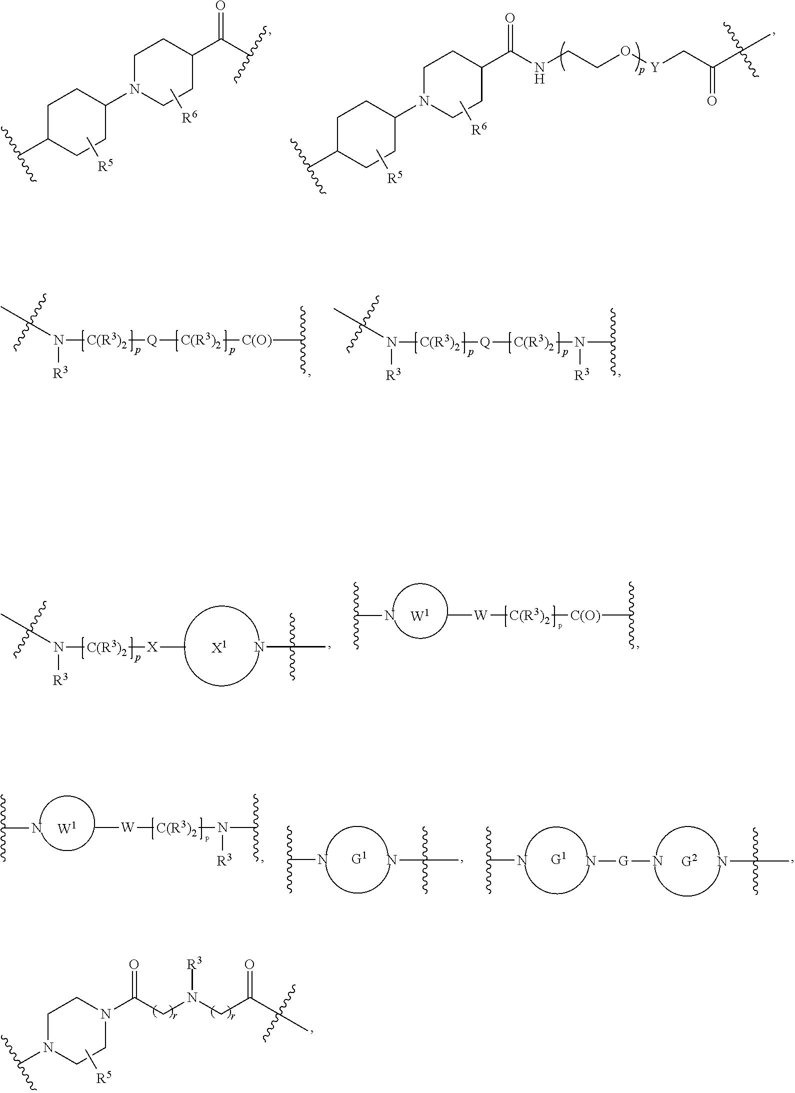

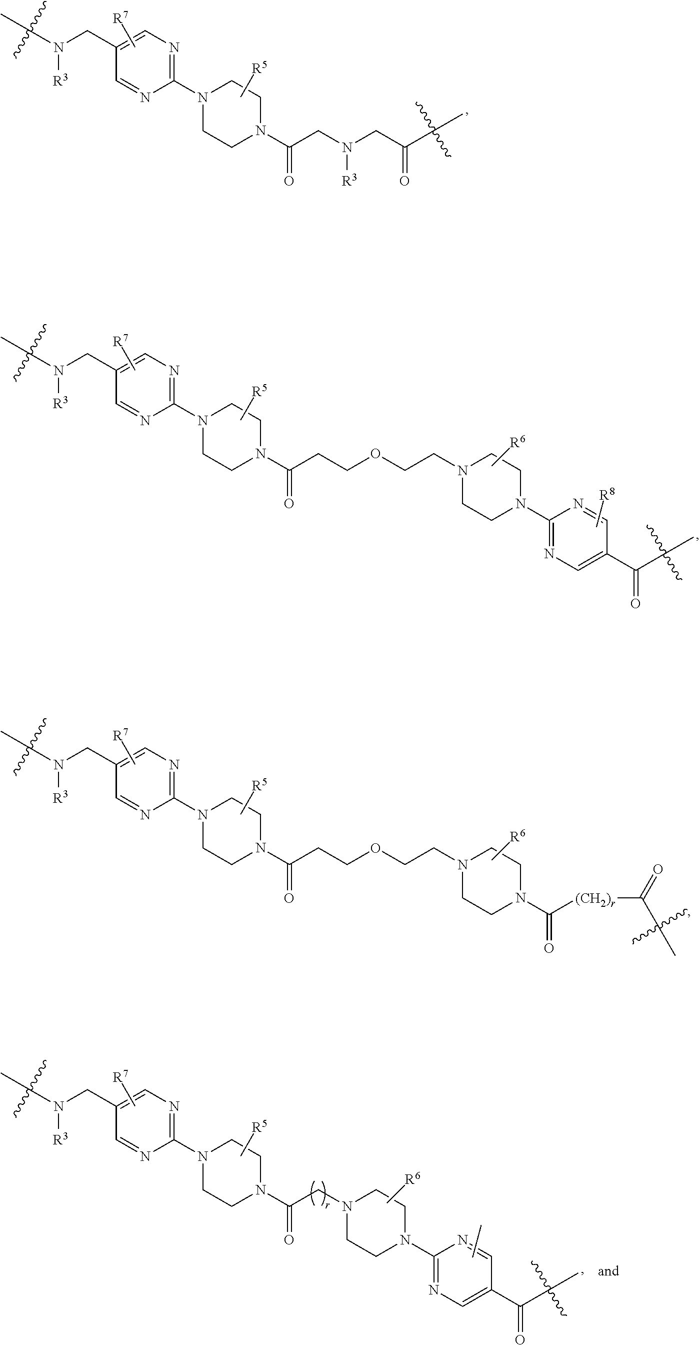

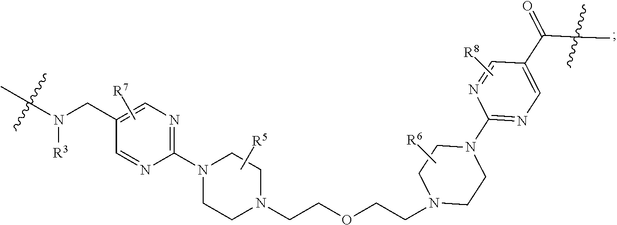

wherein the bond on the left side of A¹, as drawn, is bound to —C(═Z¹)—; and wherein the bond on the right side of the A² moiety, as drawn, is bound to B;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

each L¹ is independently selected from

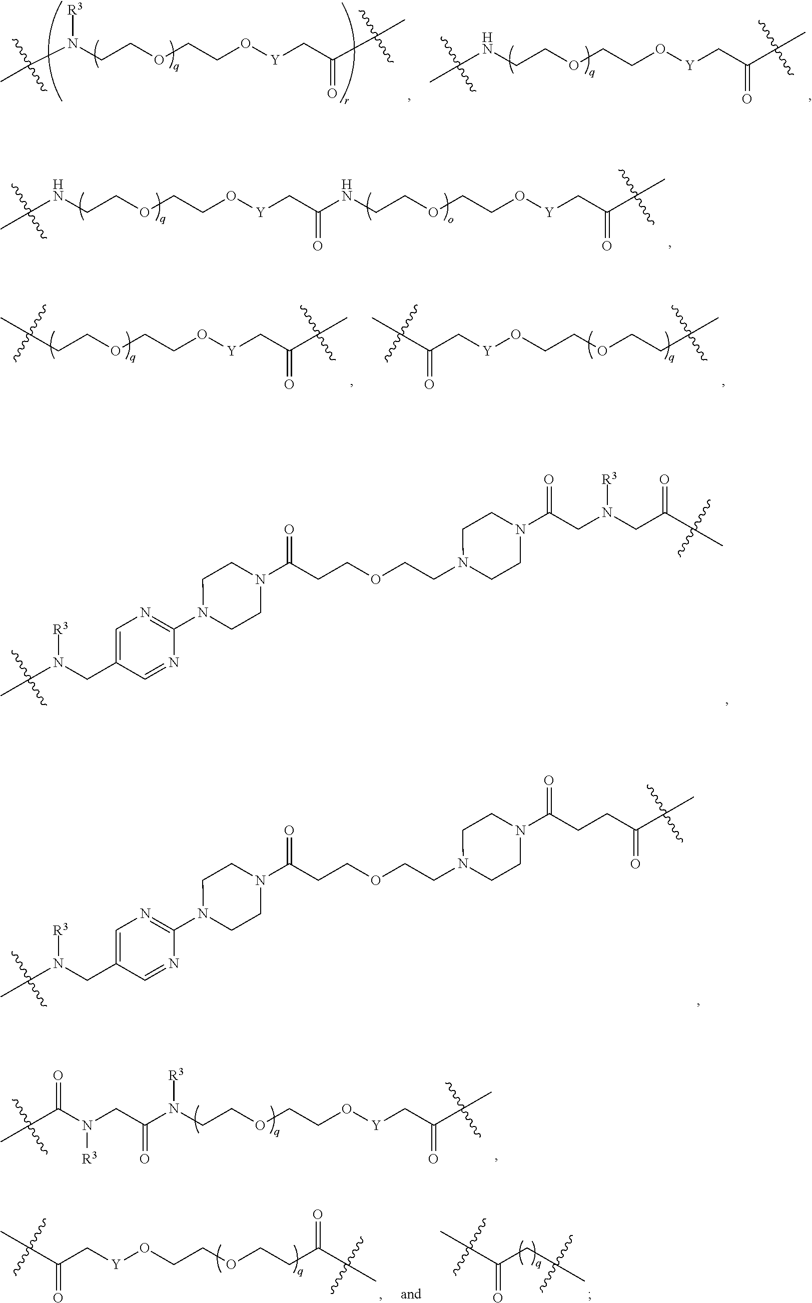

each B is independently selected from

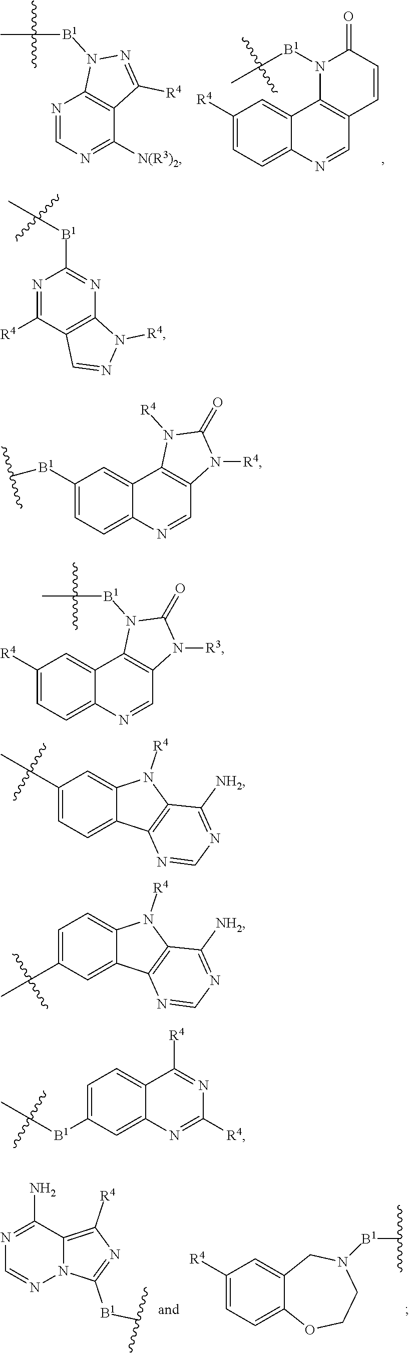

each B¹ is independently selected from

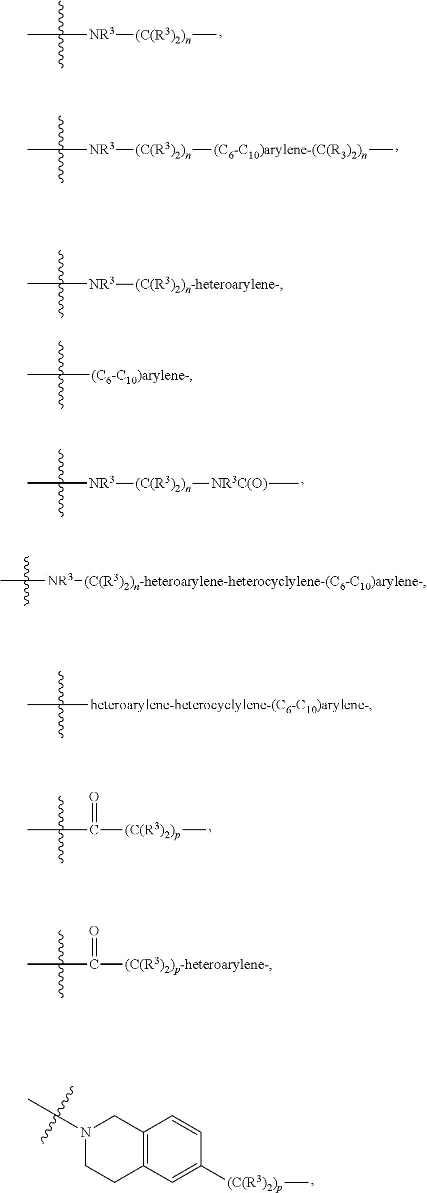

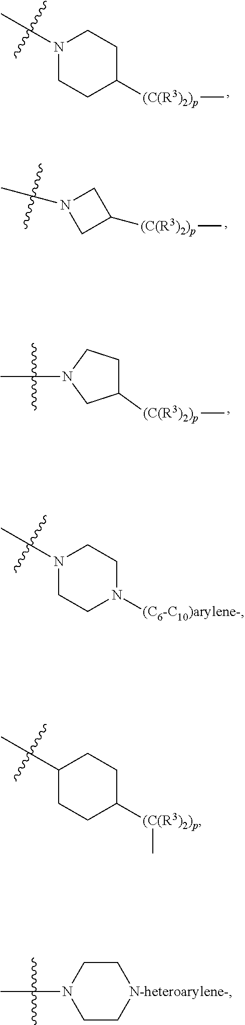

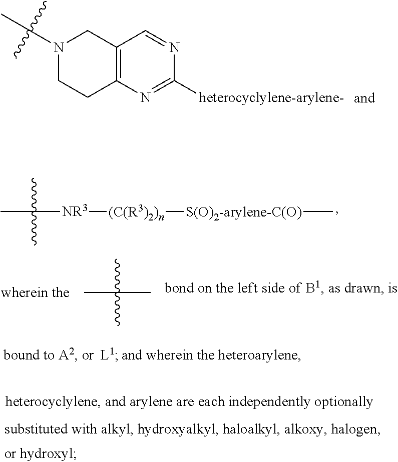

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl. —(C₁-C₆)alkylene-heteroaryl, —(C₁-C₆)alkylene-CN, —C(O)NR³-heteroaryl, or —C(O)NR³-heterocyclyl;

each R⁵ is independently H, (C₁-C₆)alkyl, —C(O)OR³, or —N(R³)₂, wherein the alkyl of (C₁-C₆)alkyl is optionally substituted with —N(R³)₂ or —OR³;

each R⁶ is independently H, (C₁-C₆)alkyl, —C(O)OR³, or —N(R³)₂, wherein the alkyl of (C₁-C₆)alkyl is optionally substituted with —N(R³)₂ or —OR³;

each R⁷ is independently H, (C₁-C₆)alkyl, —C(O)OR³, or —N(R³)₂, wherein the alkyl of (C₁-C₆)alkyl is optionally substituted with —N(R³)₂ or —OR³;

each R⁸ is independently H, (C₁-C₆)alkyl, —C(O)OR³, or —N(R³)₂, wherein the alkyl of (C₁-C₆)alkyl is optionally substituted with —N(R³)₂ or —OR³;

each Y is independently C(R³)₂ or a bond;

each n is independently an integer from one to 12;

each o is independently an integer from zero to 30;

each p is independently an integer from zero to 12;

each q is independently an integer from zero to 30; and

each r is independently an integer from one to 6.

In some embodiments, a compound of Formula I or II is represented by the structure of Formula I-28:

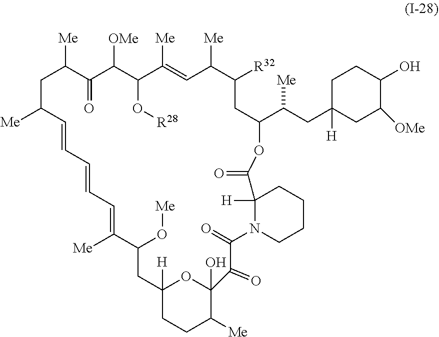

or a pharmaceutically acceptable salt or tautomer thereof.

In some embodiments, a compound of Formula Ia, Ic, I, or II is represented by the structure of Formula I-28b:

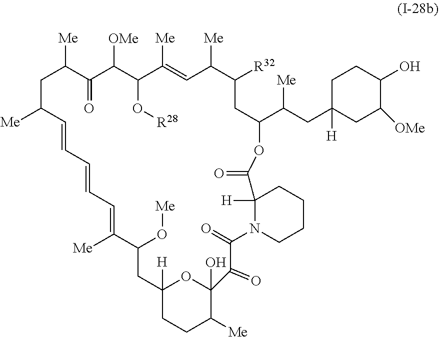

or a pharmaceutically acceptable salt or a tautomer thereof.

In some embodiments, a compound of Formula I or II is represented by the structure of Formula I-40:

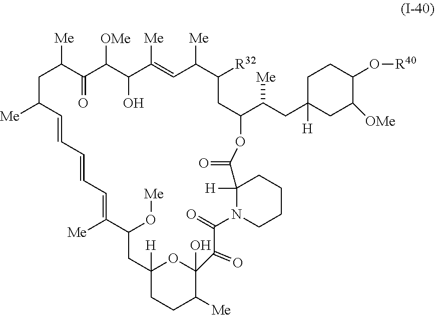

or a pharmaceutically acceptable salt or tautomer thereof.

In some embodiments, a compound of Formula Ia, Ic, I or II is represented by the structure of Formula I-40b:

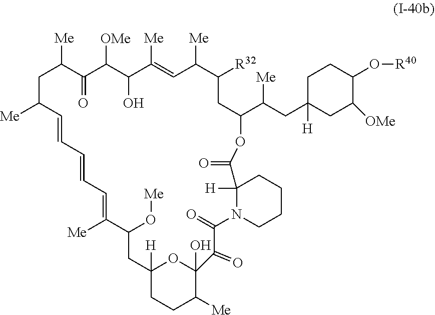

or a pharmaceutically acceptable salt or a tautomer thereof.

In some embodiments, a compound of Formula Ia, Ic, I or II is represented by the structure of Formula I-32b:

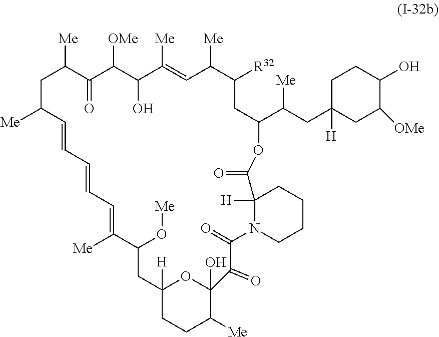

or a pharmaceutically acceptable salt or a tautomer thereof.

The present disclosure provides a method of treating a disease or disorder mediated by mTOR comprising administering to the subject suffering from or susceptible to developing a disease or disorder mediated by mTOR a therapeutically effective amount of one or more disclosed compounds. The present disclosure provides a method of preventing a disease or disorder mediated by mTOR comprising administering to the subject suffering from or susceptible to developing a disease or disorder mediated by mTOR a therapeutically effective amount of one or more disclosed compounds. The present disclosure provides a method of reducing the risk of a disease or disorder mediated by mTOR comprising administering to the subject suffering from or susceptible to developing a disease or disorder mediated by mTOR a therapeutically effective amount of one or more disclosed compounds.

Another aspect of the present disclosure is directed to a pharmaceutical composition comprising a compound of Formula I, Ia, Ib, Ic, II, or IIb, or a pharmaceutically acceptable salt or tautomer of any of the foregoing, and a pharmaceutically acceptable carrier. The pharmaceutically acceptable carrier can further comprise an excipient, diluent, or surfactant. The pharmaceutical composition can be effective for treating, preventing, or reducing the risk of a disease or disorder mediated by mTOR in a subject in need thereof.

Another aspect of the present disclosure relates to a compound of Formula I, Ia, Ib, Ic, II, or IIb, or a pharmaceutically acceptable salt or tautomer of any of the foregoing, for use in treating, preventing, or reducing the risk of a disease or disorder mediated by mTOR in a subject in need thereof.

Another aspect of the present disclosure relates to the use of a compound of Formula I, Ia, Ib, Ic, II, or IIb, or a pharmaceutically acceptable salt or tautomer of any of the foregoing, in the manufacture of a medicament for in treating, preventing, or reducing the risk of a disease or disorder mediated by mTOR in a subject in need thereof.

The present disclosure also provides compounds that are useful in inhibiting mTOR.

### DETAILED DESCRIPTION OF THE DISCLOSURE

The present disclosure relates to mTOR inhibitors. Specifically, the embodiments are directed to compounds and compositions inhibiting mTOR, methods of treating diseases mediated by mTOR, and methods of synthesizing these compounds.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the claims, the singular forms also may include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Terms

The articles “a” and “an” are used in this disclosure and refers to one or more than one (i.e., to at least one) of the grammatical object of the article, unless indicated otherwise. By way of example. “an element” may mean one element or more than one element, unless indicated otherwise.

The term “or” means “and/or” unless indicated otherwise. The term “and/or” means either “and” or “or”, or both, unless indicated otherwise.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment an optionally substituted group has 2 substituents. In another embodiment an optionally substituted group has 3 substituents. In another embodiment an optionally substituted group has 4 substituents. In another embodiment an optionally substituted group has 5 substituents.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene.” by itself or as part of another substituent, means, unless otherwise stated, a divalent radical derived from an alkyl. Typically, an alkyl (or alkylene) group will have from 1 to 24 carbon atoms, such as those groups having 10 or fewer carbon atoms.

The term “alkenyl” means an aliphatic hydrocarbon group containing a carbon-carbon double bond and which may be straight or branched having about 2 to about 6 carbon atoms in the chain. Certain alkenyl groups have 2 to about 4 carbon atoms in the chain. Branched may mean that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkenylene,” by itself or as part of another substituent, means, unless otherwise stated, a divalent radical derived from an alkene.

The term “alkynyl” means an aliphatic hydrocarbon group containing a carbon-carbon triple bond and which may be straight or branched having about 2 to about 6 carbon atoms in the chain. Certain alkynyl groups have 2 to about 4 carbon atoms in the chain. Branched may mean that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

The term “alkynylene,” by itself or as part of another substituent, means, unless otherwise stated, a divalent radical derived from an alkyne.

The term “cycloalkyl” means a monocyclic or polycyclic saturated or partially unsaturated carbon ring containing 3-18 carbon atoms. Examples of cycloalkyl groups include, without limitation, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₅ cycloalkyl is a cycloalkyl group containing between 3 and 8 carbon atoms. A cycloalkyl group can be fused (e.g., decalin) or bridged (e.g., norbornane).

A “cycloalkylene,” alone or as part of another substituent, means a divalent radical derived from a cycloalkyl.

The terms “heterocyclyl” or “heterocycloalkyl” or “heterocycle” refers to a monocyclic or polycyclic 3 to 24-membered ring containing carbon and at least one heteroatom selected from oxygen, phosphorous, nitrogen, and sulfur and wherein there is not delocalized n electrons (aromaticity) shared among the ring carbon or heteroatom(s). Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g., can be a bicyclic ring.

A “heterocyclylene” or “heterocycloalkylene.” alone or as part of another substituent, means a divalent radical derived from a “heterocyclyl” or “heterocycloalkyl” or “heterocycle.”

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl may refer to multiple rings fused together wherein at least one of the fused rings is an aryl ring.

An “arylene,” alone or as part of another substituent, means a divalent radical derived from an aryl.

The term “heteroaryl” refers to an aryl group (or rings) that contains at least one heteroatom such as N, O, or S, wherein the nitrogen and sulfur atom(s) are optionally oxidized, and the nitrogen atom(s) is optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described herein.

The term “heteroaryl” may also include multiple condensed ring systems that have at least one such aromatic ring, which multiple condensed ring systems are further described below. The term may also include multiple condensed ring systems (e.g., ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1, 2, 3, 4-tetrahydro-1,8-naphthyridinyl), carbocycles (to form for example 5,6,7,8-tetrahydroquinolyl) and aryls (to form for example indazolyl) to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen).

A “heteroarylene,” alone or as part of another substituent, means a divalent radical derived from a heteroaryl.

Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

The terms “halo” or “halogen.” by themselves or as part of another substituent, mean, unless otherwise stated, a fluorine, chlorine, bromine, or iodine atom. Additionally, terms such as “haloalkyl” may include monohaloalkyl and polyhaloalkyl. For example, the term “halo(C₁-C₄)alkyl” may include, but is not limited to, fluoromethyl, difluoromethyl, trifluoromethyl, 2,2,2-trifluoroethyl, 4-chlorobutyl, 3-bromopropyl, 1-fluoro-2-bromoethyl, and the like.

The term “hydroxyl.” as used herein, means —OH.

The term “hydroxyalkyl” as used herein, means an alkyl moiety as defined herein, substituted with one or more, such as one, two or three, hydroxy groups. In certain instances, the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl.

The term “oxo,” as used herein, means an oxygen that is double bonded to a carbon atom.

A substituent group, as used herein, may be a group selected from the following moieties:

(A) oxo, halogen, —CF₃, —CN, —OH, —OCH₃, —NH₂, —COOH, —CONH₂, —NO₂, —SH, —SO₃H. —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —OCH₂F, unsubstituted alkyl, unsubstituted cycloalkyl, unsubstituted heterocycloalkyl, unsubstituted aryl, unsubstituted heteroaryl, and
(B) alkyl, cycloalkyl, heterocyclyl, aryl, heteroaryl, substituted with at least one substituent selected from:

(i) oxo, halogen, —CF₃, —CN, —OH, —OCH₃, —NH₂, —COOH, —CONH₂, —NO₂, —SH. —SO₃H, —SO₄H, —SO₂NH₂, —NHNH₂, —ONH₂, —NHC═(O)NHNH₂, —NHC═(O)NH₂, —NHSO₂H. —NHC═(O)H, —NHC(O)—OH, —NHOH, —OCF₃, —OCHF₂, —OCH₂F, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, heteroaryl, substituted with at least one substituent selected from:
(a) oxo, halogen, —CF₃, —CN, —OH, —OCH₃, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂. —NHSO₂H, —NHC═(O)H, —NHC(O)—OH, —NHOH, —OCF₃, —OCHF₂, —OCH₂F, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH. —OCH₃, —NH₂, —COOH, —CONH₂, —NO₂, —SH, —SO₃H, —SO₄H, —SO₂NH₂, —NHNH₂. —ONH₂, —NHC═(O)NHNH₂, —NHC═(O)NH₂, —NHSO₂H, —NHC═(O)H, —NHC(O)—OH, —NHOH, —OCF₃, —OCHF₂, —OCH₂F, unsubstituted alkyl, unsubstituted heteroalkyl, unsubstituted cycloalkyl, unsubstituted heterocycloalkyl, unsubstituted aryl, unsubstituted heteroaryl.

An “effective amount” when used in connection with a compound is an amount effective for treating or preventing a disease in a subject as described herein.

The term “carrier”, as used in this disclosure, encompasses carriers, excipients, and diluents and may mean a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating may include curing, improving, or at least partially ameliorating the disorder.

The term “prevent” or “preventing” with regard to a subject refers to keeping a disease or disorder from afflicting the subject. Preventing may include prophylactic treatment. For instance, preventing can include administering to the subject a compound disclosed herein before a subject is afflicted with a disease and the administration will keep the subject from being afflicted with the disease.

The term “disorder” is used in this disclosure and means, and is used interchangeably with, the terms disease, condition, or illness, unless otherwise indicated.

The term “administer”. “administering”, or “administration” as used in this disclosure refers to either directly administering a disclosed compound or pharmaceutically acceptable salt or tautomer of the disclosed compound or a composition to a subject, or administering a prodrug derivative or analog of the compound or pharmaceutically acceptable salt or tautomer of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds

The present disclosure provides a compound having the structure of Formula Ic,

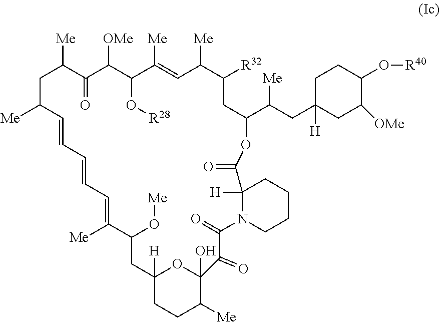

or a pharmaceutically acceptable salt or tautomer thereof, wherein R³², R²⁸, and R⁴⁰ are described as above.

The present disclosure provides a compound having the structure of Formula Ia,

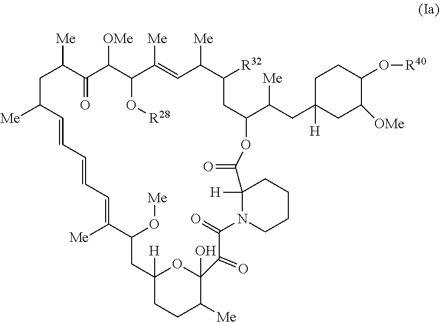

or a pharmaceutically acceptable salt or tautomer thereof, wherein R³², R²⁸, and R⁴⁰ are described as above.

The present disclosure provides a compound having the structure of Formula I,

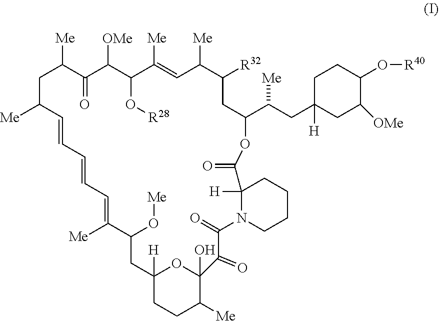

or a pharmaceutically acceptable salt or tautomer thereof, wherein R³², R²⁸, and R⁴⁰ are described as above.

The resent disclosure provides a compound having the structure of Formula Ib:

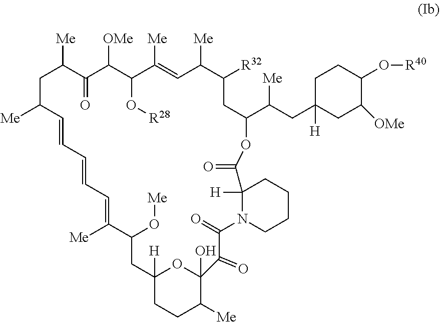

or a pharmaceutically acceptable salt and or tautomer thereof, wherein R³², R²⁸, and R⁴⁰ are described as above for Formula I.

The present disclosure provides a compound having the structure of Formula II,

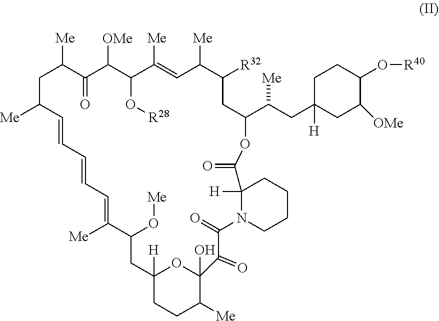

or a pharmaceutically acceptable salt or tautomer thereof, wherein R³², R²⁸, and R⁴⁰ are described as above.

The present disclosure provides a compound having the structure of Formula IIb:

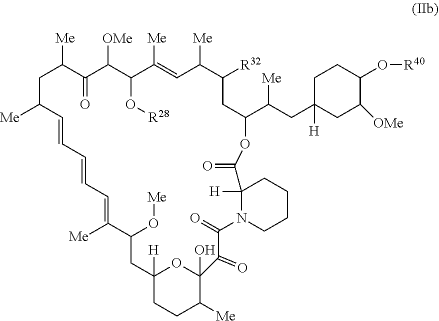

or a pharmaceutically acceptable salt or tautomer thereof, wherein R³², R²⁸, and R⁴⁰ are described as above for Formula II.

In certain embodiments, a compound has the following formula:

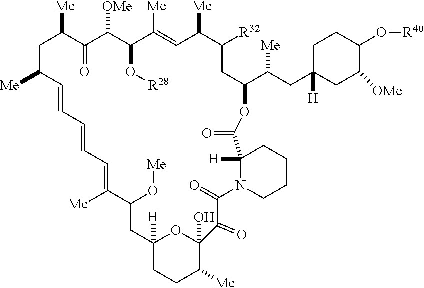

or a pharmaceutically acceptable salt or tautomer thereof.

In certain embodiments, R³² is ═O. In certain embodiments, R³² is —OR³. In certain embodiments, R³² is H. In certain embodiments, R³² is —N₃.

As described above, each R³ is independently H or (C₁-C₆)alkyl. In certain embodiments, R³ is H. In certain embodiments, R³ is (C₁-C₆)alkyl. In certain embodiments, R³ is methyl.

In certain embodiments, R²⁸ is H. In certain embodiments, R²⁸ is (C₁-C₆)alkyl. In certain embodiments, R⁴⁰ is H.

In certain embodiments, a compound is represented by the structure of Formula I-40b:

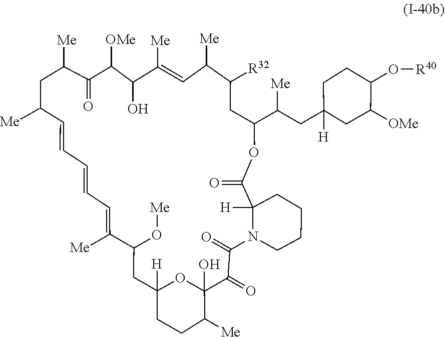

or a pharmaceutically acceptable salt or tautomer thereof, wherein R³² and R⁴⁰ are described as above for Formula Ia, Ic, I, or II.

In certain embodiments, a compound is represented by the structure of Formula I-40:

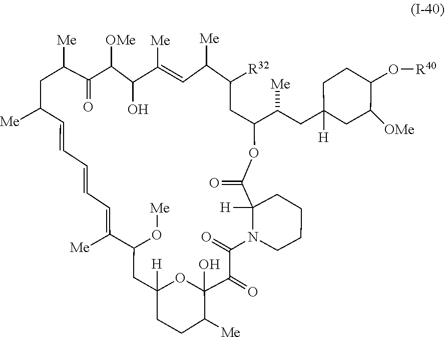

or a pharmaceutically acceptable salt or tautomer thereof, wherein R³² and R⁴⁰ are described as above.

In certain embodiments, R⁴⁰ is —C(═Z¹)—R^{40a}. In certain embodiments, Z¹ is O. In certain embodiments, Z¹ is S.

In certain embodiments, R is —O—(C₁-C₆)alkyl or —O—(C₆-C₁₀)aryl; wherein the aryl is unsubstituted or substituted with 1-5 substituents selected from NO₂ and halogen.

In certain embodiments, R^{40a} is -A¹-L¹-A²-B. In certain embodiments, R^{40a} is -A¹-A²-B. In certain embodiments, R^{40a} is -L²-A¹-L¹-A²-L³-B.

In certain embodiments, R^{40a} is -A¹-L-A²-B, wherein A¹ and A² are absent. In certain embodiments, R^{40a} is -A¹-L¹-A²-B, wherein A² is absent. In certain embodiments, R^{40a} is -A¹-L¹-A²-B, wherein A¹ is absent. In certain embodiments, R^{40a} is -A¹-L¹-A²-B. In certain embodiments, R^{40a} is -A¹-A²-B. In certain embodiments, R^{40a} is -L²-A¹-L¹-A²-L³-B, wherein L² and A¹ are absent. In certain embodiments, R^{40a} is -L²-A¹-L²-A²-L³-B, wherein L² is absent. In certain embodiments, R^{40a} is -L²-A¹-L¹-A²-L³-B, wherein L³ is absent.

In certain embodiments, a compounds is represented by the structure of Formula I-28b:

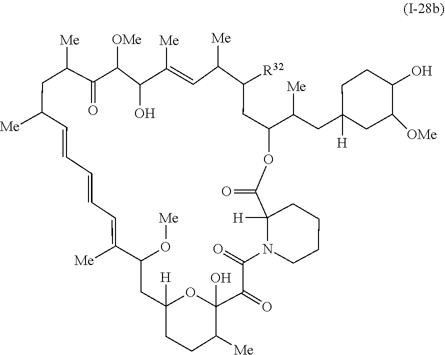

or a pharmaceutically acceptable salt or tautomer thereof, wherein R³² and R²⁸ are described as above for Formula Ia, Ic, I, or II.

In certain embodiments, a compound is represented by the structure of Formula I-28:

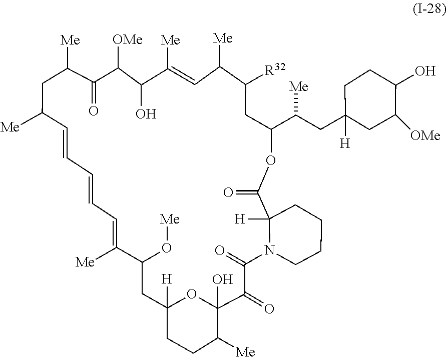

or a pharmaceutically acceptable salt or tautomer thereof, wherein R³² and R²⁸ are described as above.

In certain embodiments, R²⁸ is —C(═Z¹)—R^{28a}. In certain embodiments, Z¹ is O. In certain embodiments, Z¹ is S.

In certain embodiments, R^{28a} is —O—(C₁-C₆)alkyl or —O—(C₆-C₁₀)aryl; wherein the aryl is unsubstituted or substituted with 1-5 substituents selected from NO₂ and halogen.

In certain embodiments, R^{28a} is -A¹-L¹-A²-B. In certain embodiments, R^{28a} is -A¹-A²-B. In certain embodiments, R^{28a} is -L²-A¹-L¹-A²-L³-B.

In certain embodiments, R^{28a} is -A¹-L¹-A²-B, wherein A¹ and A² are absent. In certain embodiments, R^{28a} is -A¹-L¹-A²-B, wherein A² is absent. In certain embodiments, R^{28a} is -A¹-L¹-A²-B, wherein A¹ is absent. In certain embodiments, R^{28a} is -A¹-L¹-A²-B. In certain embodiments, R^{28a} is -A¹-A²-B. In certain embodiments, R^{28a} is -L²-A¹-L¹-A²-L³-B, wherein L² and A¹ are absent. In certain embodiments, R^{28a} is -L²-A¹-L¹-A²-L³-B, wherein L² is absent. In certain embodiments, R^{28a} is -L²-A¹-L¹-A²-L³-B, wherein L³ is absent.

In certain embodiments, the compounds are represented by the structure of Formula I-32b:

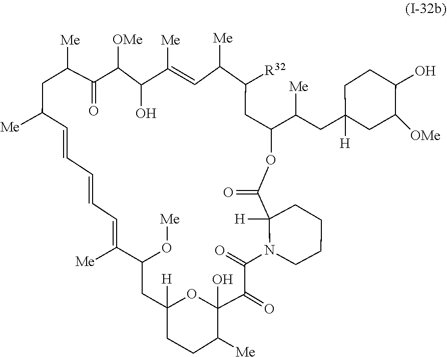

or a pharmaceutically acceptable salt or tautomer thereof, wherein R³² is described as above for Formula Ia, Ic, I, or II.

In certain embodiments, R³² is —O—C(═Z¹)—R^{32a}. In certain embodiments, Z¹ is O. In certain embodiments, Z¹ is S.

In certain embodiments, R^{32a} is —O—(C₁-C₆)alkyl or —O—(C₆-C₁₀)aryl; wherein the aryl is unsubstituted or substituted with 1-5 substituents selected from NO₂ and halogen.

In certain embodiments, R^{32a} is -A¹-L¹-A²-B. In certain embodiments, R^{32a} is -A¹-A²-B. In certain embodiments, R^{32a} is -L²-A¹-L¹-A²-L³-B.

In certain embodiments, R^{32a} is -A¹-L¹-A²-B, wherein A¹ and A² are absent. In certain embodiments, R^{32a} is -A¹-L¹-A²-B, wherein A² is absent. In certain embodiments, R^{32a} is -A¹-L¹-A²-B, wherein A¹ is absent. In certain embodiments, R^{32a} is -A¹-L¹-A²-B. In certain embodiments, R^{32a} is -A¹-A²-B. In certain embodiments, R^{32a} is -L²-A¹-L¹-A²-L³-B, wherein L² and A¹ are absent. In certain embodiments, R^{32a} is -L²-A¹-L¹-A²-L³-B, wherein L² is absent. In certain embodiments, R^{32a} is -L²-A¹-L¹-A²-L³-B, wherein L³ is absent.

As described above, each L¹ is independently selected from

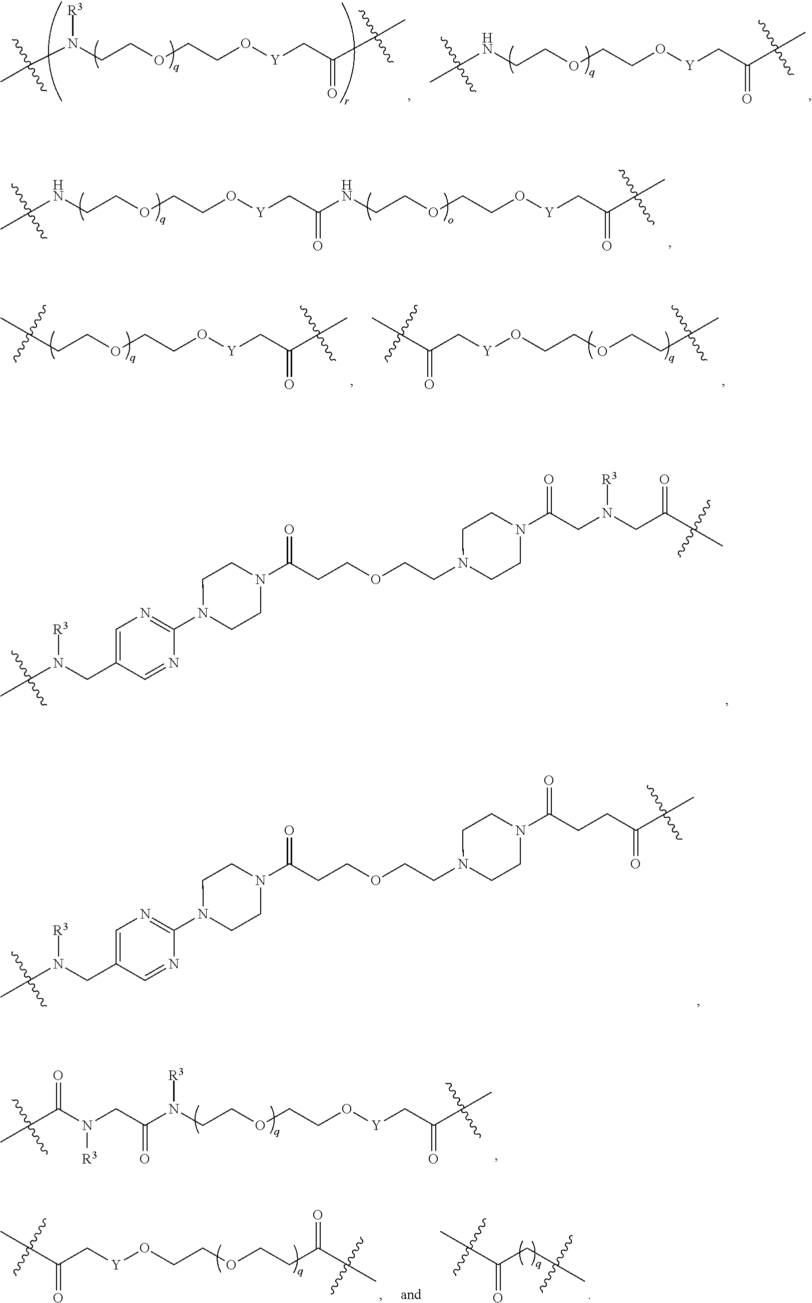

As described above for Formula Ia, each L¹ is independently selected from

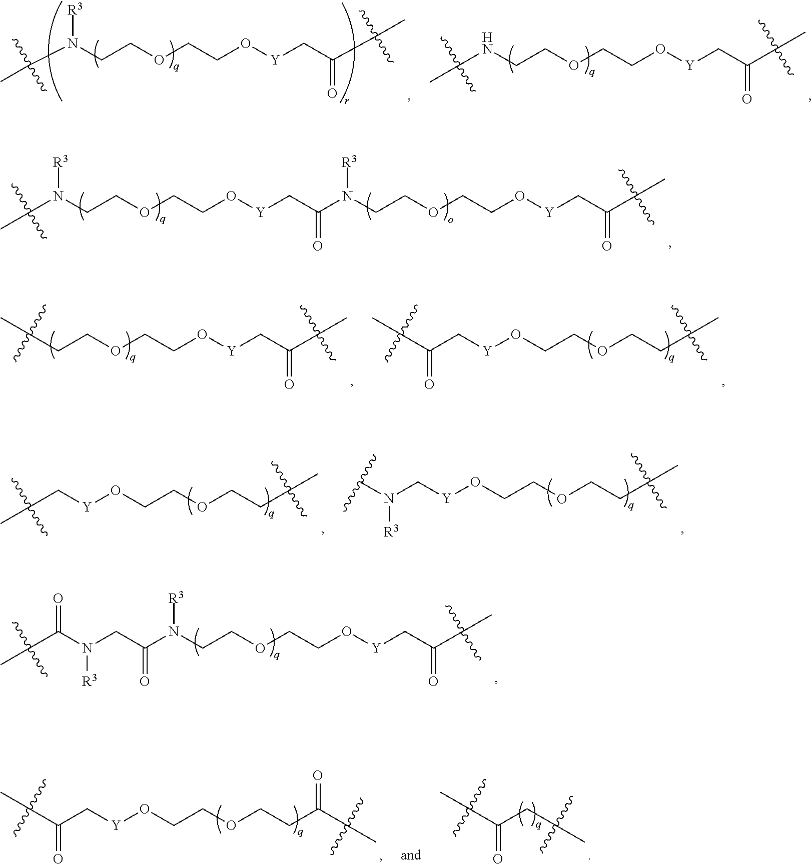

As described above for Formula Ic, each L¹ is independently selected from

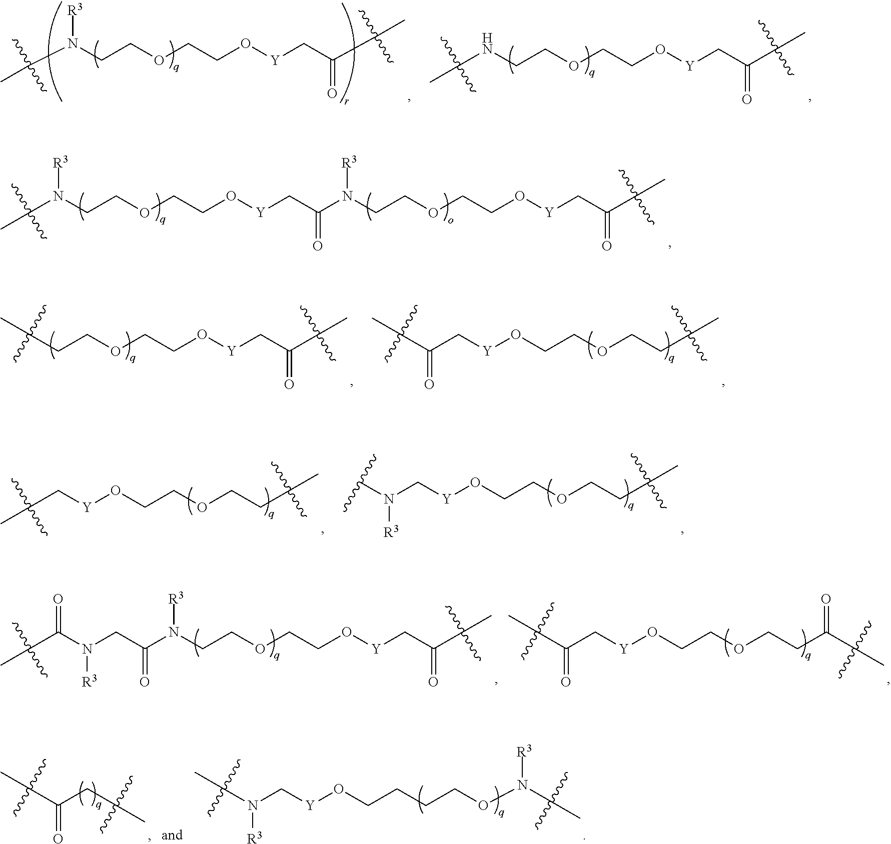

In certain embodiments, L¹ is O

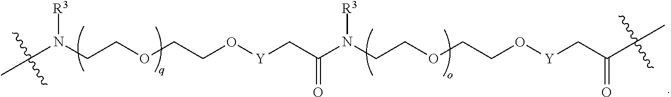

In certain embodiments, L¹ is

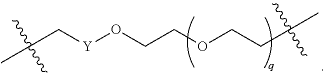

In certain embodiments, L¹ is

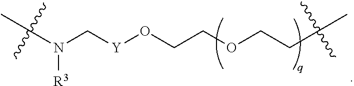

In certain embodiments, L¹ is

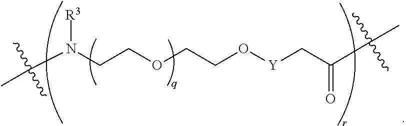

In certain embodiments, L¹ is

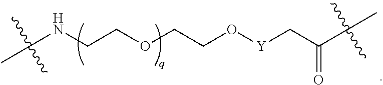

In certain embodiments, L¹ is

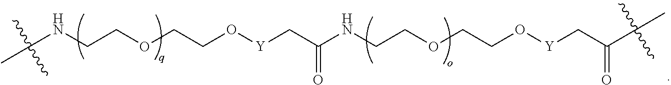

In certain embodiments, L¹ is

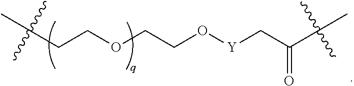

In certain embodiments, L¹ is

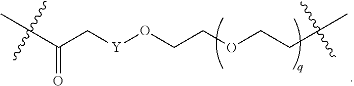

In certain embodiments, L¹ is

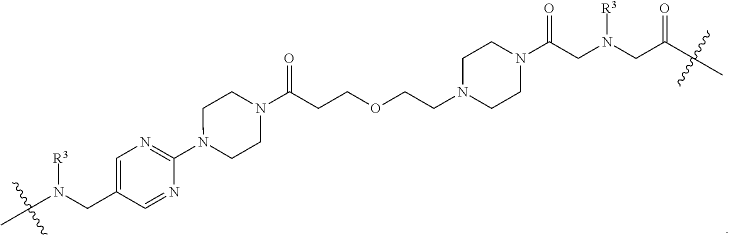

In certain embodiments, L¹ is

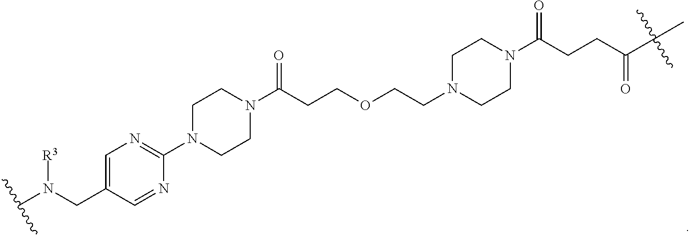

In certain embodiments, L¹ is

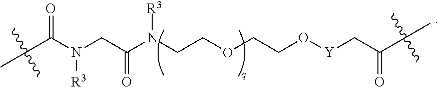

In certain embodiments, L¹ is

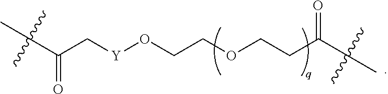

In certain embodiments, L¹ is

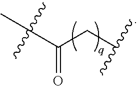

In certain embodiments, L¹ is

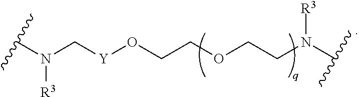

As described above. L² and L³ are independently absent or are independently selected from

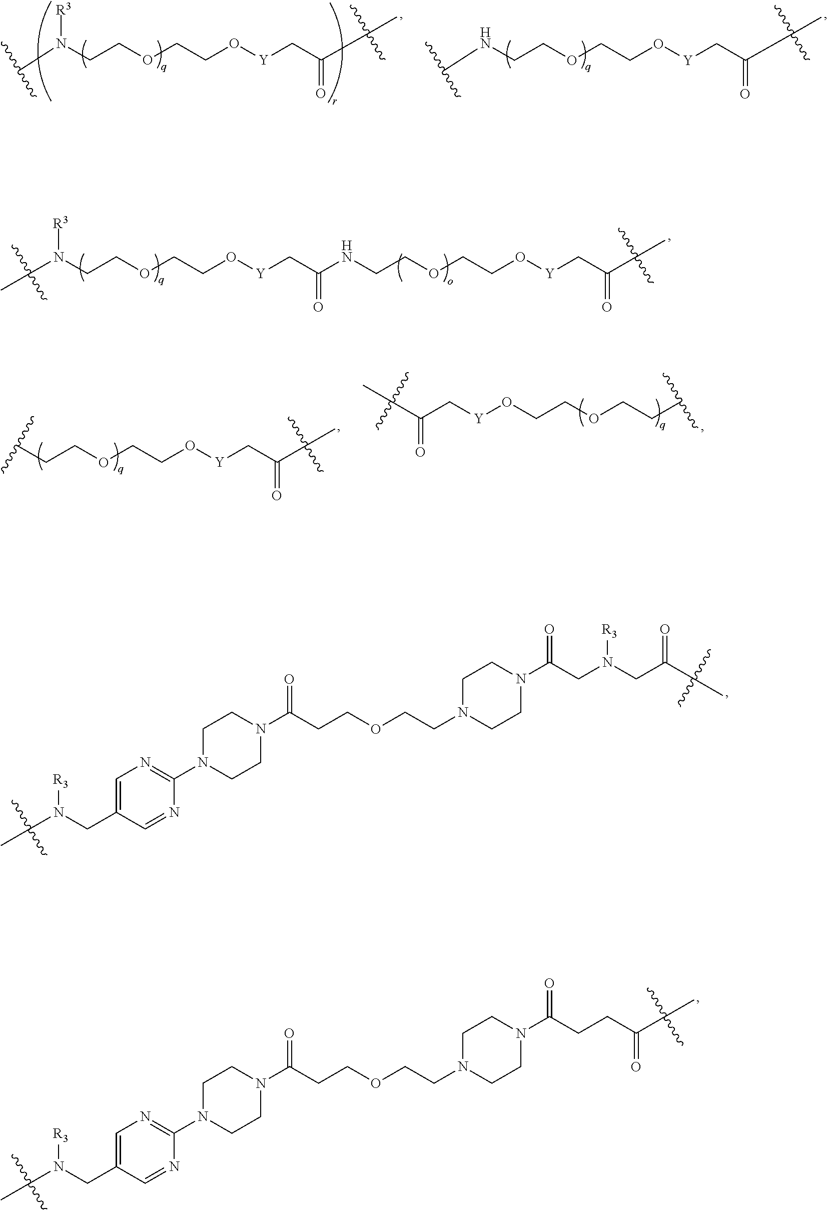

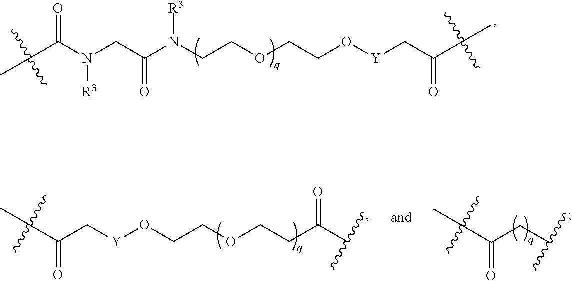

As described above for Formula Ia and Ic, L² and L³ are independently absent or are independently selected from

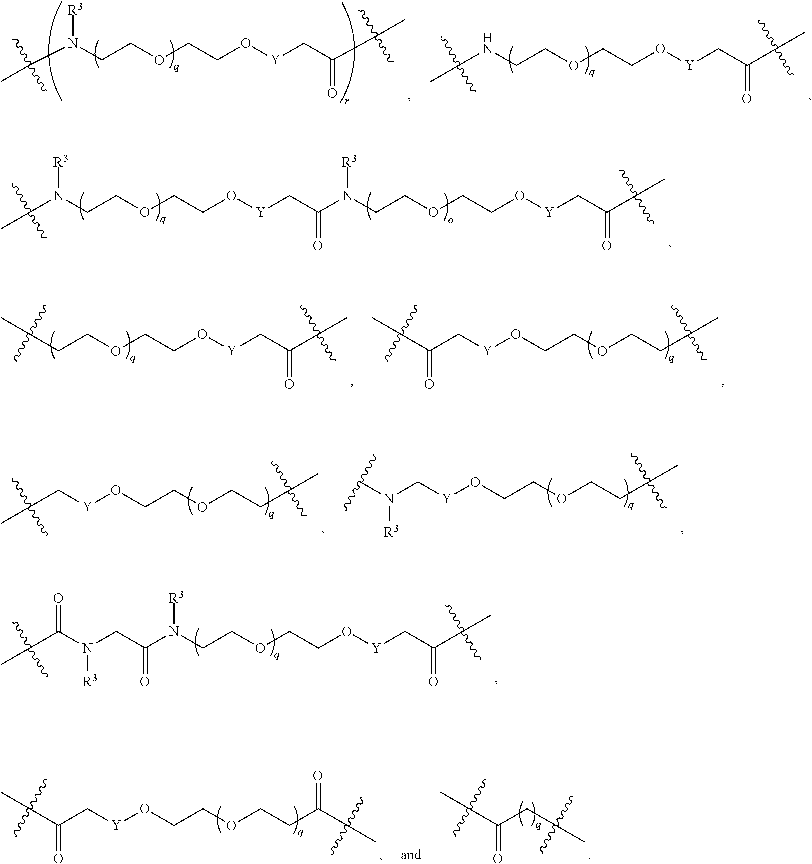

In certain embodiments, L² is absent. In certain embodiments, L² is

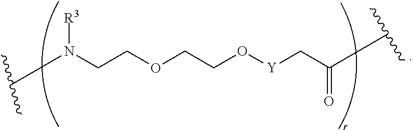

In certain embodiments, L² is

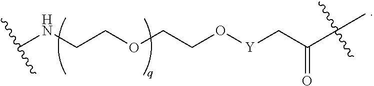

In certain embodiments, L² is

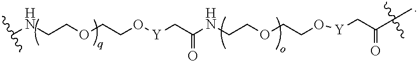

In certain embodiments, L² is

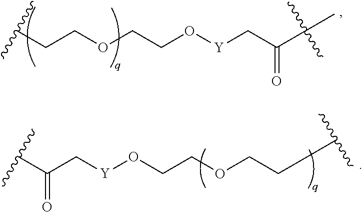

In certain embodiments, L² is

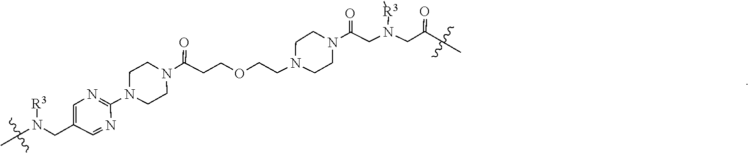

In certain embodiments, L² is

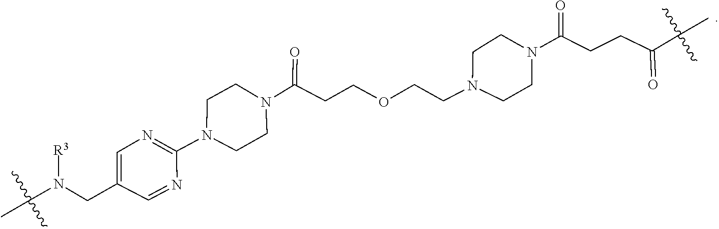

In certain embodiments, L² is

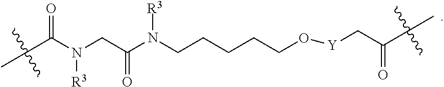

In certain embodiments, L² is

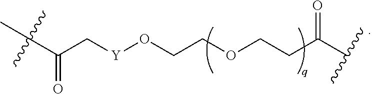

In certain embodiments, L² is

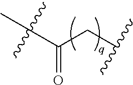

In certain embodiments, L² is

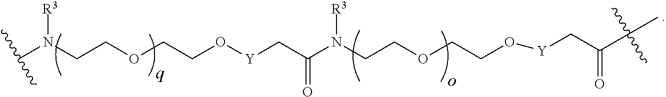

In certain embodiments, L² is

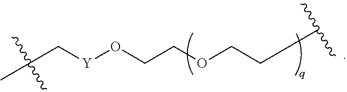

In certain embodiments, L² is

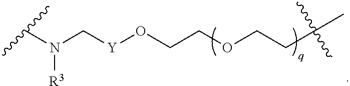

In certain embodiments, L³ is absent. In certain embodiments, L³ is

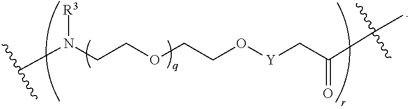

In certain embodiments, L³ is

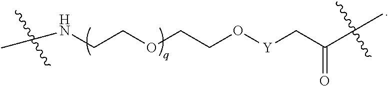

In certain embodiments, L³ is

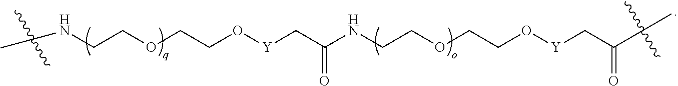

In certain embodiments, L³ is

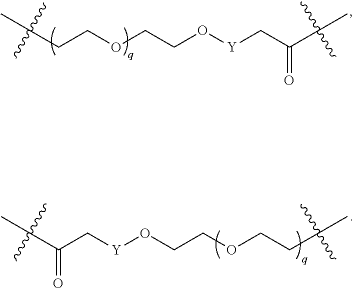

In certain embodiments, L³ is

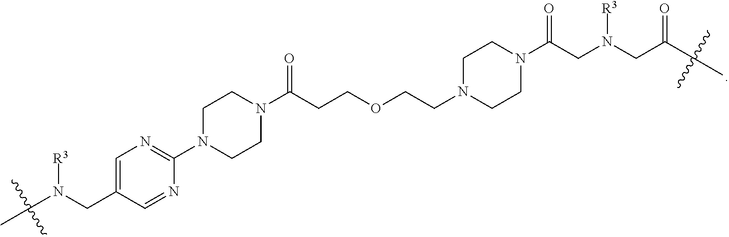

In certain embodiments, L³ is

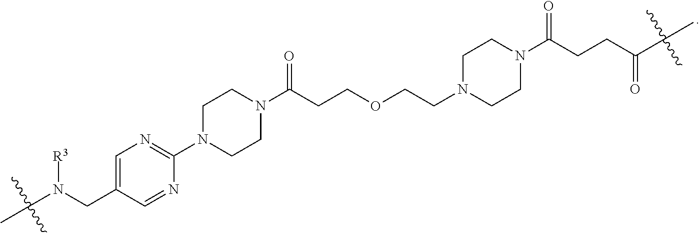

In certain embodiments, L³ is

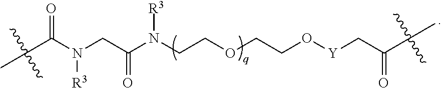

In certain embodiments, L³ is

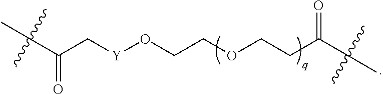

In certain embodiments, L³ is In certain embodiments, L² is

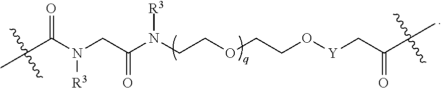

In certain embodiments, L² is

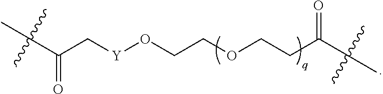

In certain embodiments, L² is

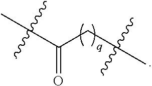

In certain embodiments, L³ is

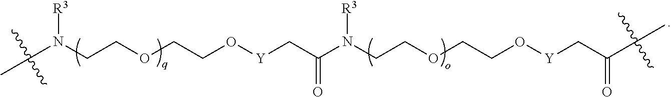

In certain embodiments, L³ is

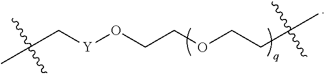

In certain embodiments, L³ is

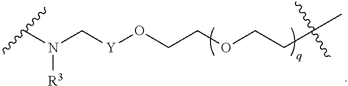

As described above, A¹ and A² are independently absent or are independently selected from

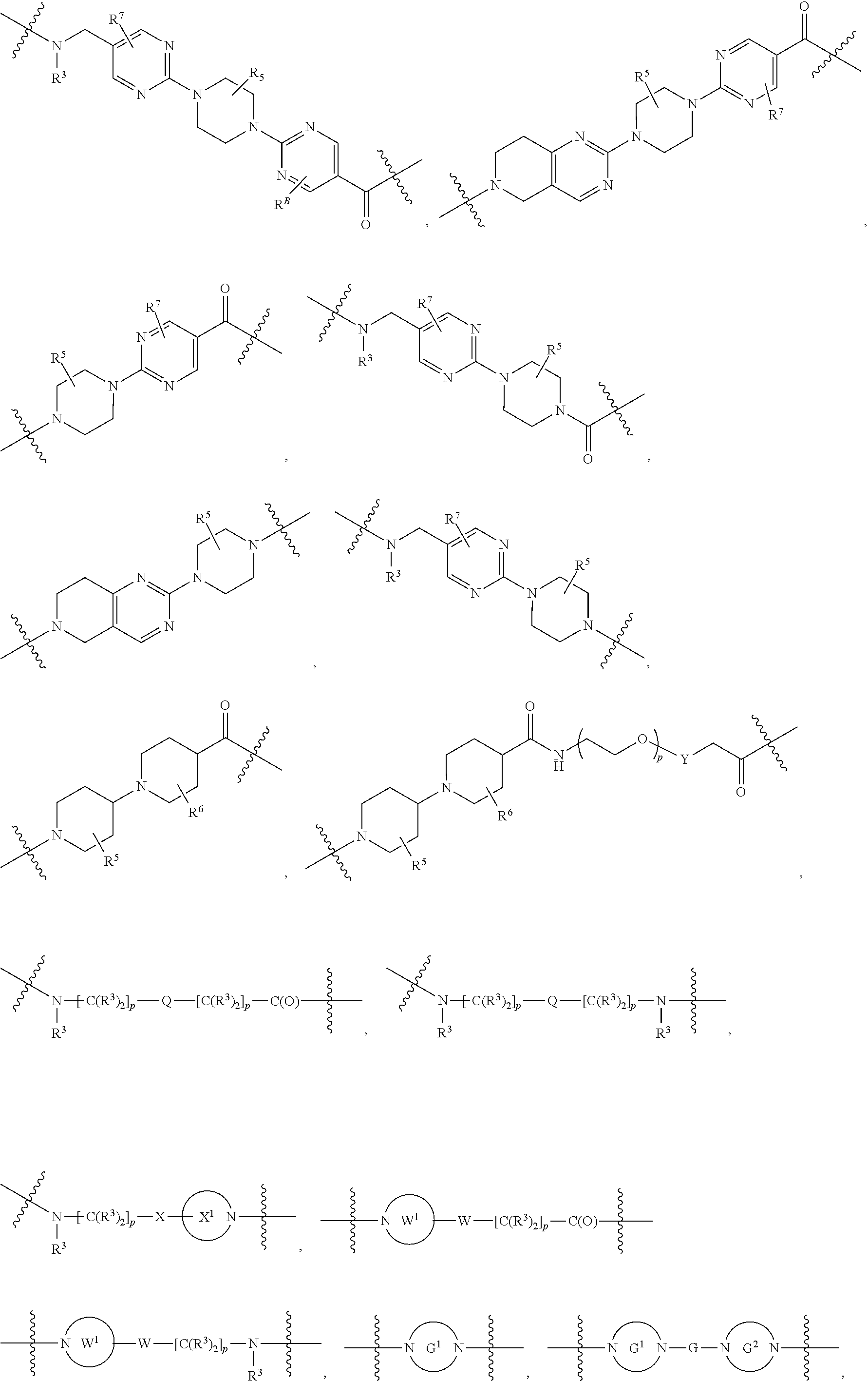

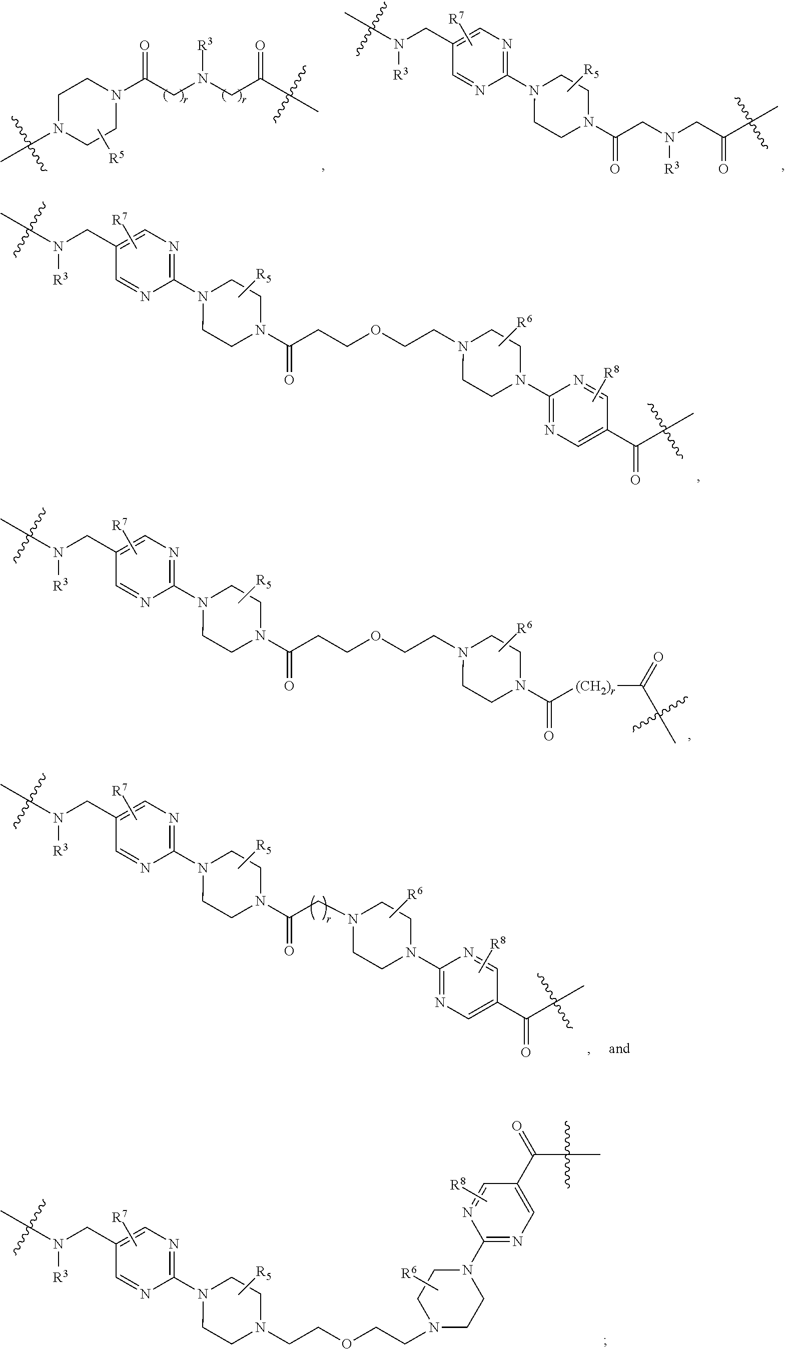

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring;

each G is independently absent or a ring selected from arylene, cycloalkylene, heteroarylene, and heterocyclylene;

each G¹ and G² are independently heteroarylene or heterocyclylene ring.

As described above for Formula Ia, A¹ and A² are independently absent or are independently selected from

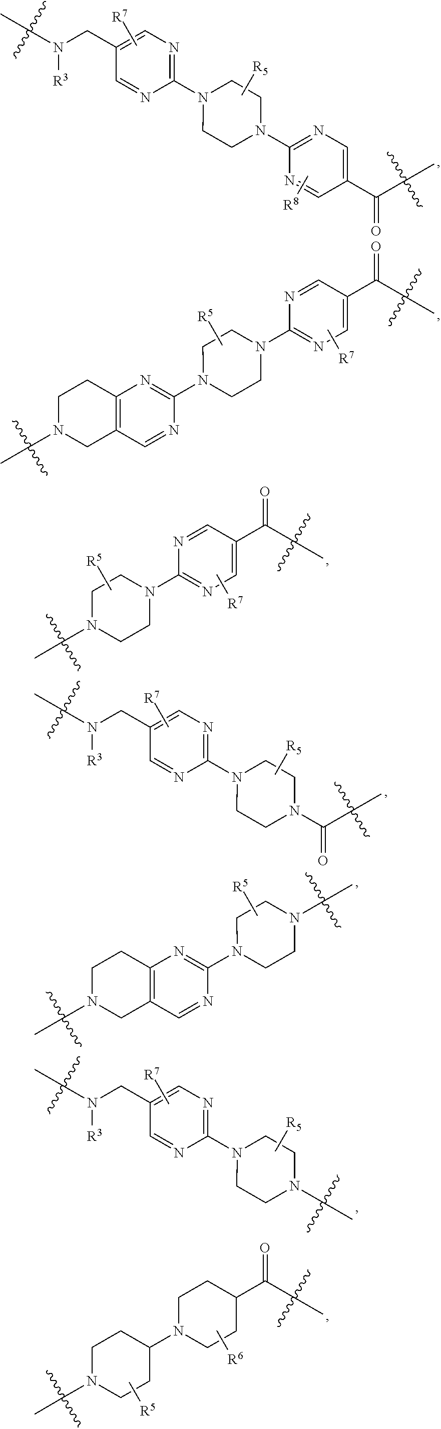

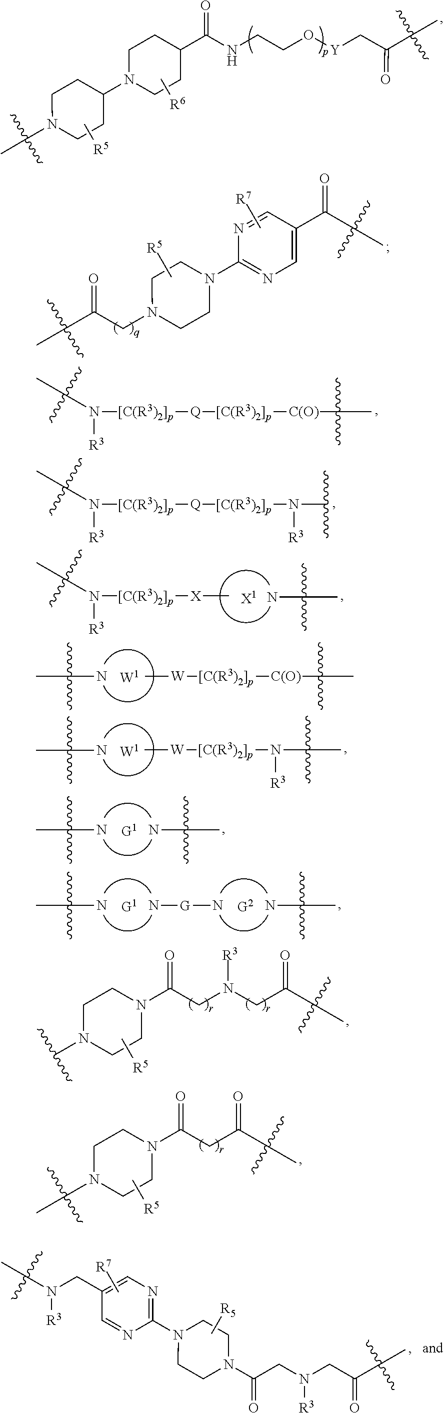

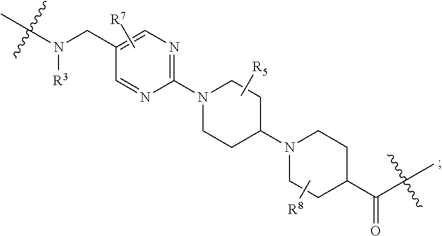

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring;

each G is independently absent or a ring selected from arylene, cycloalkylene, heteroarylene, and heterocyclylene;

each G¹ and G² are independently heteroarylene or heterocyclylene ring.

As described above for Formula Ic, A¹ and A² are independently absent or are independently selected from

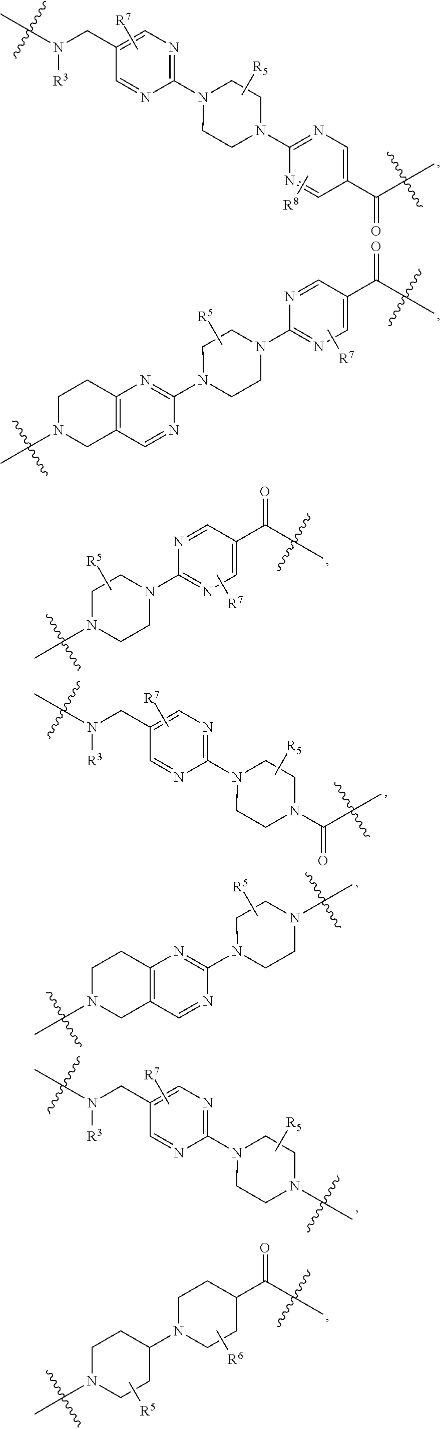

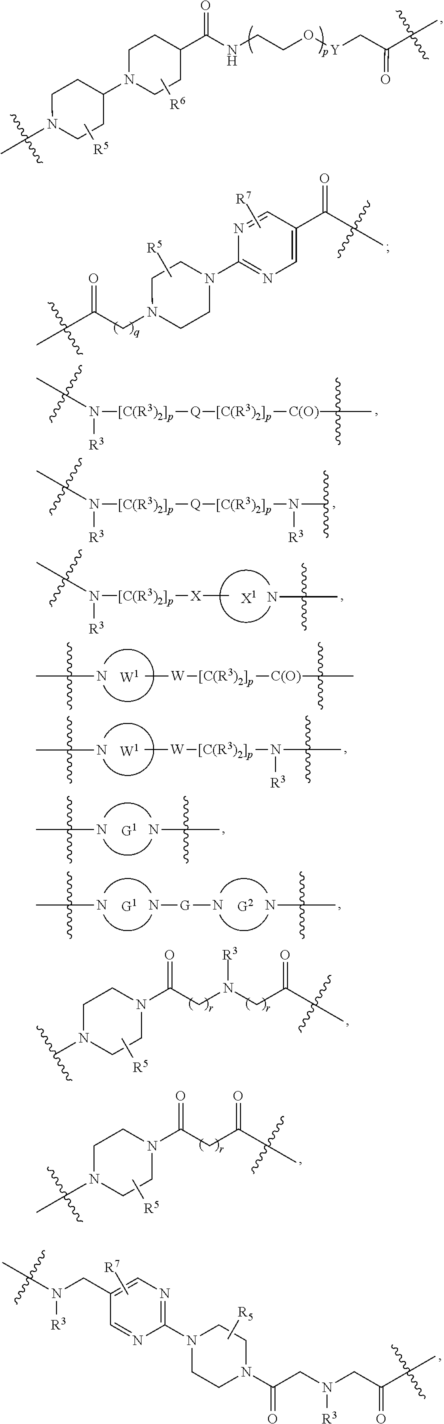

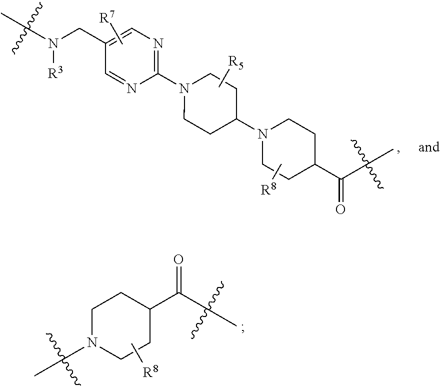

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring;

each G is independently absent or a ring selected from arylene, cycloalkylene, heteroarylene, and heterocyclylene;

each G¹ and G² are independently heteroarylene or heterocyclylene ring:

For Formula I, the bond on the left side of A¹, as drawn, is bound to —C(═Z¹)— or L²; and the bond on the right side of the A² moiety, as drawn, is bound to B or L³. For Formula II, the bond on the left side of A¹, as drawn, is bound to —C(═Z¹)—; and the bond on the right side of the A² moiety, as drawn, is bound to B. For Formula Ia and Ic, the bond on the left side of A¹, as drawn, is bound to —C(═Z¹)— or L²; and wherein the bond on the right side of the A² moiety, as drawn, is bound to B or L³.

In certain embodiments, A¹ is absent. In certain embodiments, A¹ is

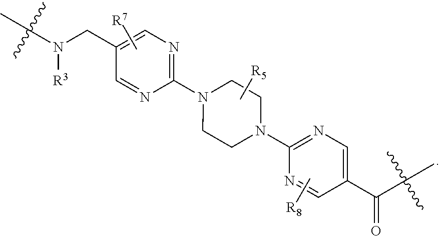

In certain embodiments, A¹ is

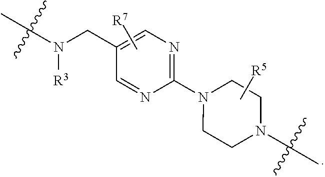

In certain embodiments, A¹ is

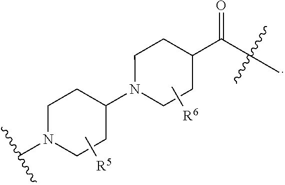

In certain embodiments, A¹ is

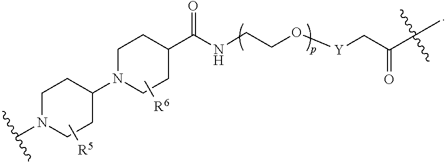

In certain embodiments, A¹ is

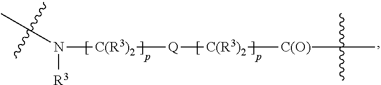

wherein each Q is independently 1 to 3 rings selected from arylene, cycloalkylene, heteroarylene, and heterocyclylene.

In certain embodiments, A¹ is

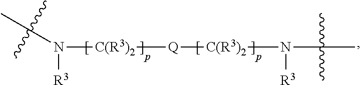

wherein each Q is independently 1 to 3 rings selected from arylene, cycloalkylene, heteroarylene, and heterocyclylene.

In certain embodiments, A¹ is

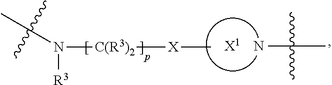

wherein each X is independently absent or 1 to 2 rings selected from arylene, cycloalkylene, heteroarylene, and heterocyclylene; and each X¹ is a heteroarylene or heterocyclylene ring.

In certain embodiments, A¹ is

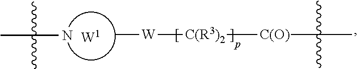

wherein each W is independently absent or 1 to 2 rings selected from arylene, cycloalkylene, heteroarylene, and heterocyclylene; and each W¹ is a heteroarylene or heterocyclylene ring.

In certain embodiments, A¹ is

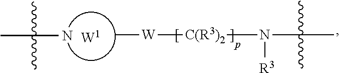

wherein each W is independently absent or 1 to 2 rings selected from arylene, cycloalkylene, heteroarylene, and heterocyclylene; and each W¹ is a heteroarylene or heterocyclylene ring.

In certain embodiments, A¹ is

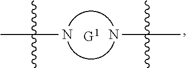

wherein each G is independently absent or a ring selected from arylene, cycloalkylene, heteroarylene, and heterocyclylene.

In certain embodiments, A¹ is

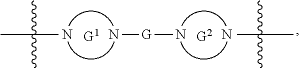

wherein each G is independently absent or a ring selected from arylene, cycloalkylene, heteroarylene, and heterocyclylene; and each G¹ and G² are independently heteroarylene or heterocyclylene ring.

In certain embodiments, A¹ is

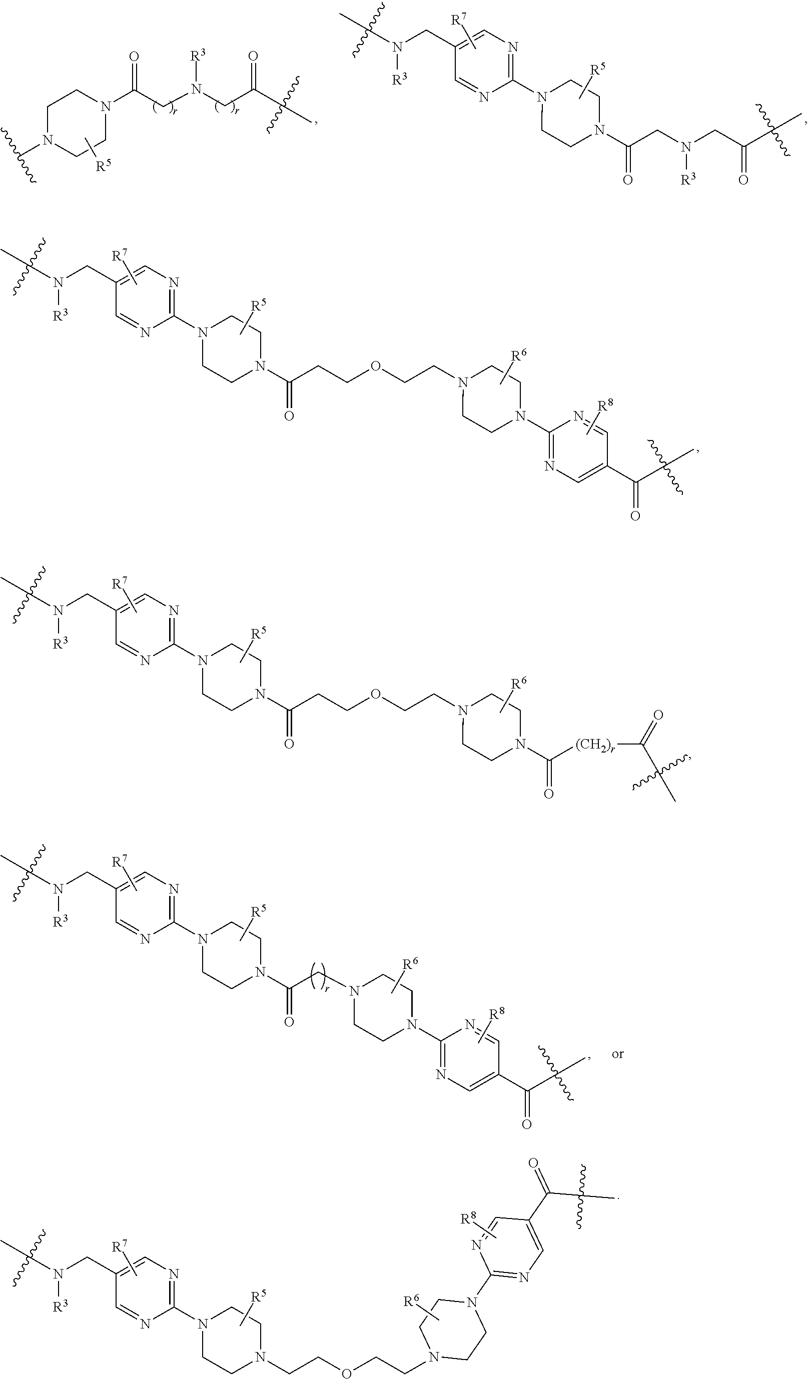

In certain embodiments, A¹ is

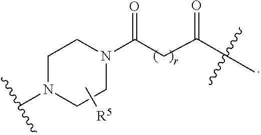

In certain embodiments, A¹ is

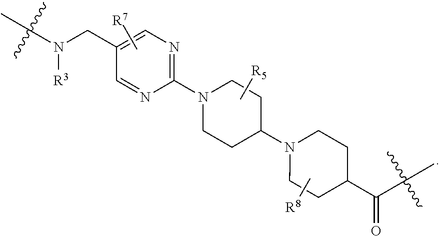

In certain embodiments, A¹ is

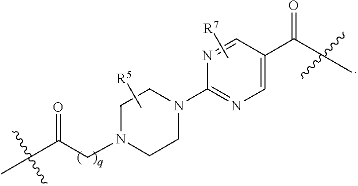

In certain embodiments, A¹ is

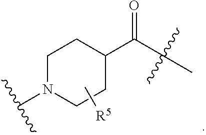

In certain embodiments, A² is absent. In certain embodiments, A² is

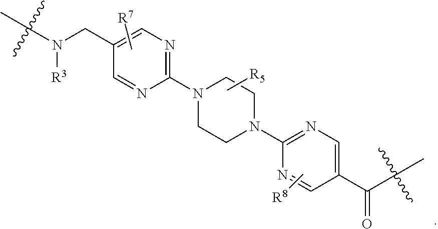

In certain embodiments, A² is

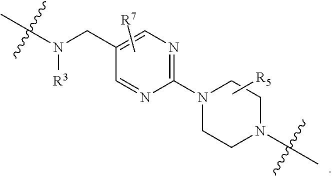

In certain embodiments, A² is

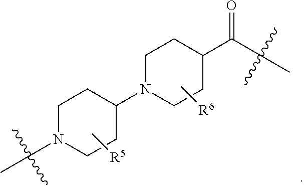

In certain embodiments, A² is

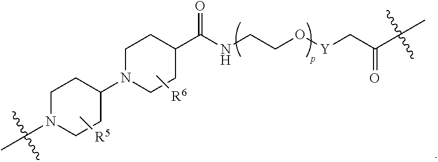

In certain embodiments, A² is

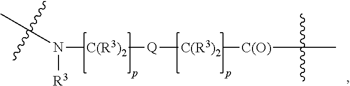

wherein each Q is independently 1 to 3 rings selected from arylene, cycloalkylene, heteroarylene, and heterocyclylene.

In certain embodiments, A² is

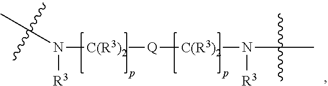

wherein each Q is independently 1 to 3 rings selected from arylene, cycloalkylene, heteroarylene, and heterocyclylene.

In certain embodiments, A² is

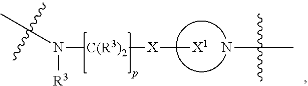

wherein each X is independently absent or 1 to 2 rings selected from arylene, cycloalkylene, heteroarylene, and heterocyclylene; and each X¹ is independently a heteroarylene or heterocyclylene ring.

In certain embodiments, A² is

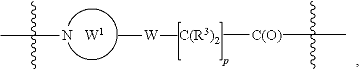

wherein each W is independently absent or 1 to 2 rings selected from arylene, cycloalkylene, heteroarylene, and heterocyclylene; and each W¹ is independently a heteroarylene or heterocyclylene ring.

In certain embodiments, A² is

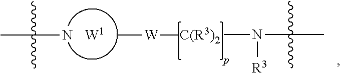

wherein each W is independently absent or 1 to 2 rings selected from arylene, cycloalkylene, heteroarylene, and heterocyclylene; and each W¹ is independently a heteroarylene or heterocyclylene ring.

In certain embodiments, A² is

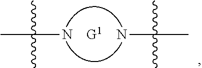

wherein each G is independently absent or a ring selected from arylene, cycloalklene, heteroarylene, and heterocyclylene.

In certain embodiments, A² is

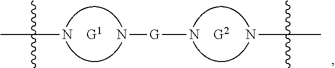

wherein each G is independently absent or a ring selected from arylene, cycloalkylene, heteroarylene, and heterocyclylene; and each G¹ and G² are independently a heteroarylene or heterocyclylene ring.

In certain embodiments, A²

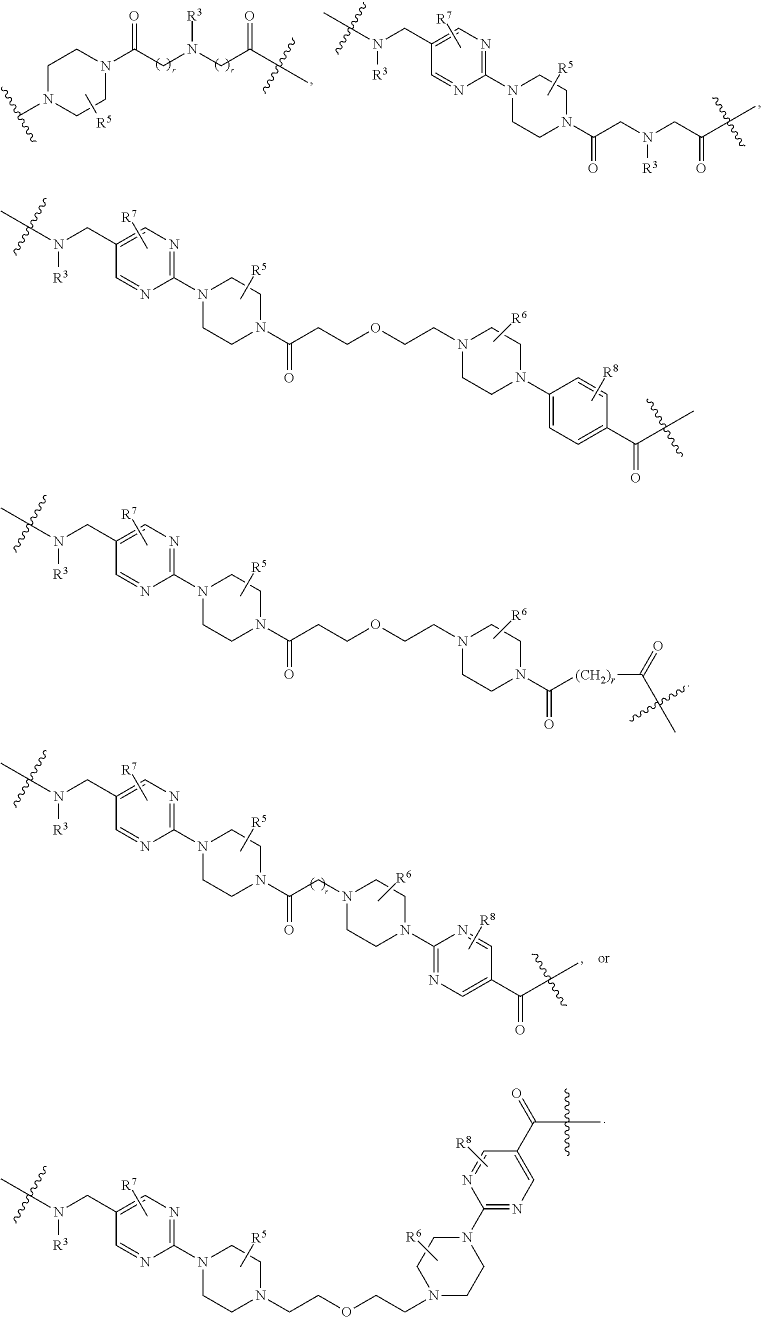

In certain embodiments, A² is

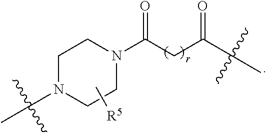

In certain embodiments, A² is

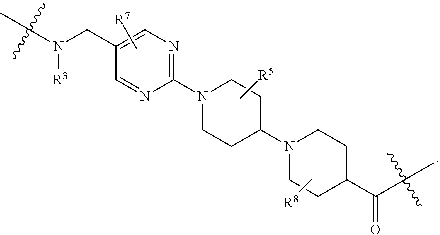

In certain embodiments, A² is

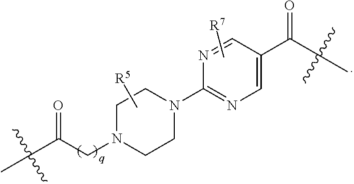

In certain embodiments, A² is

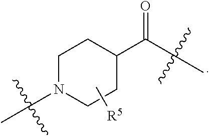

As described above, each B is independently selected from

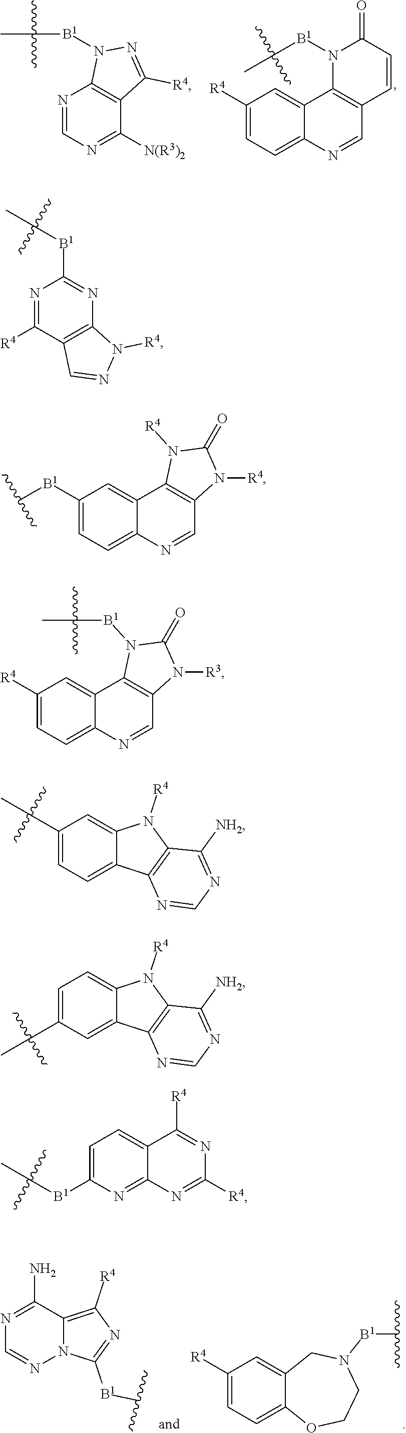

In certain embodiments, B is

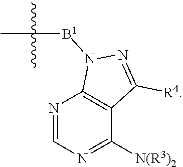

In certain embodiments, B is

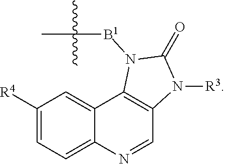

In certain embodiments, B is

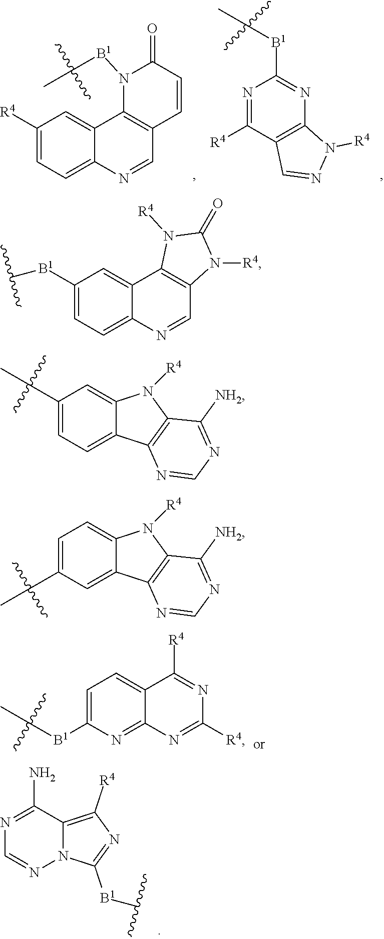

In certain embodiments, B is

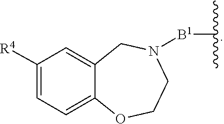

As described above, each B¹ is independently selected from

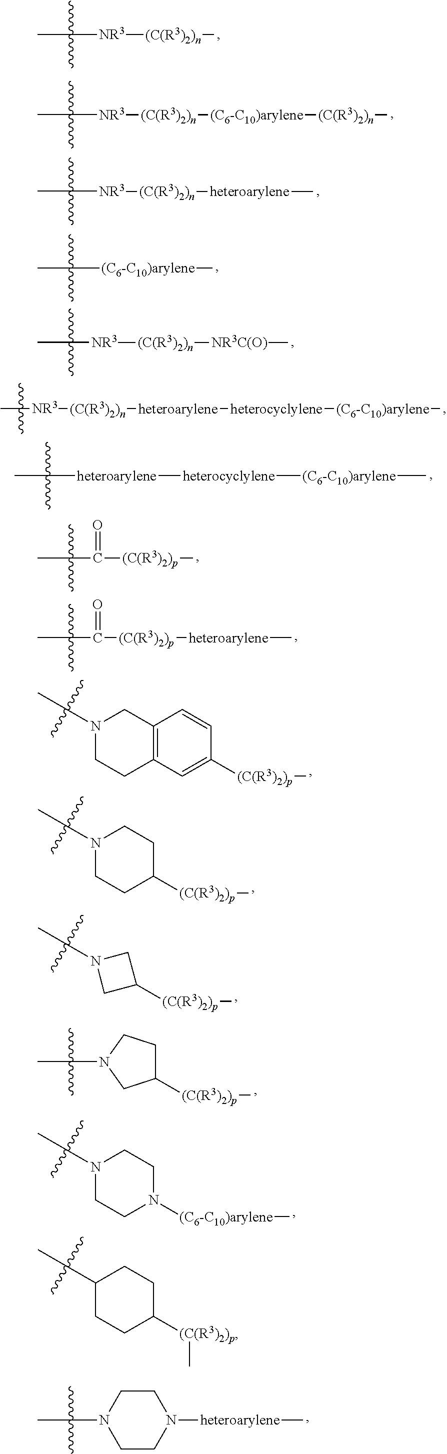

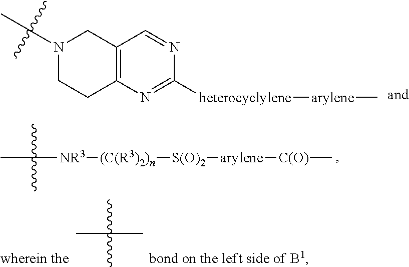

As described above for Formula Ic, each B¹ is independently selected from

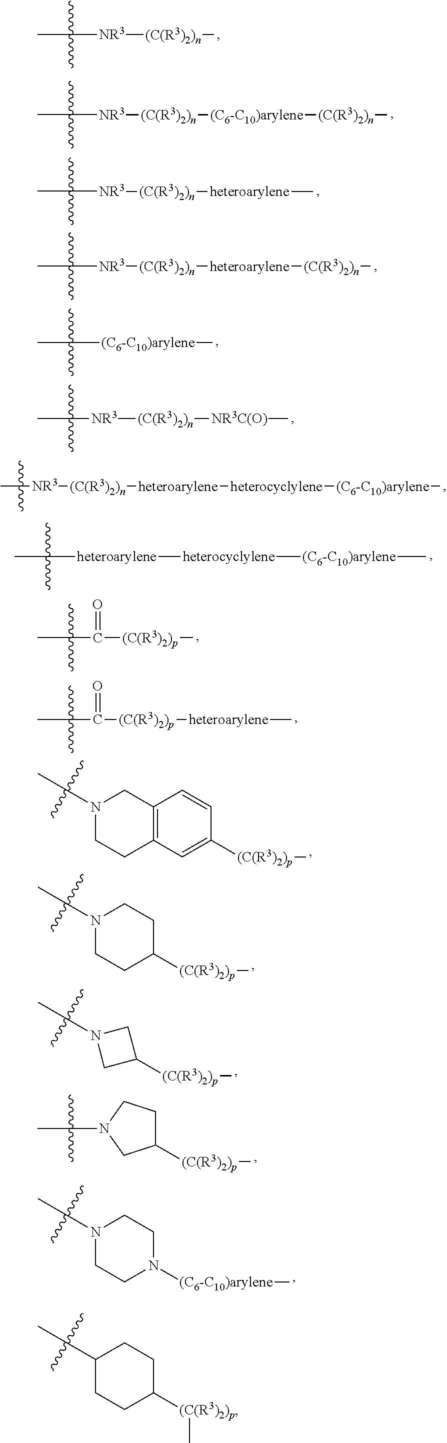

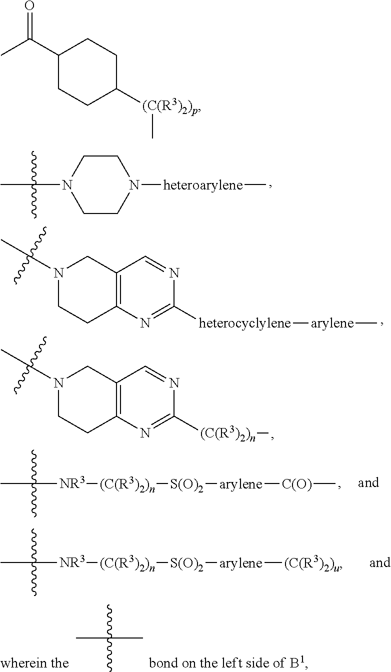

In certain embodiments, B¹ is

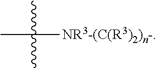

In certain embodiments, B¹ is

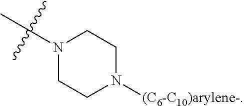

In certain embodiments, B¹ is

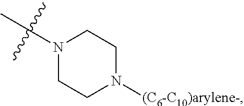

wherein arylene is optionally substituted with haloalkyl.

In certain embodiments, B¹ is

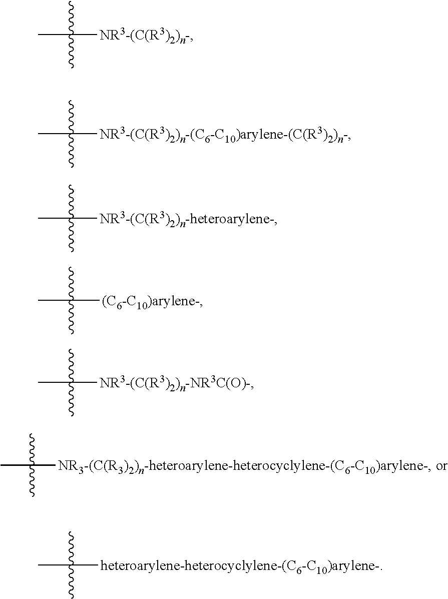

In certain embodiments, B¹ is

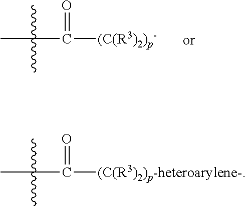

In certain embodiments, B¹ is

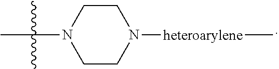

In certain embodiments, B¹ is

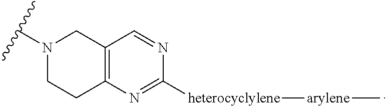

In certain embodiments, B¹ is

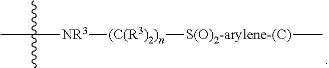

In certain embodiments, B¹ is

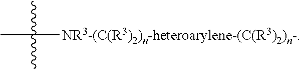

In certain embodiments, B¹ is

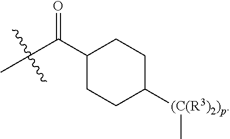

In certain embodiments, B¹ is

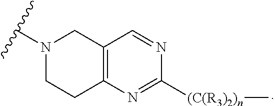

In certain embodiments, B¹ is

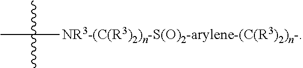

In certain embodiments, in B¹, the heteroaryl, heterocyclyl, and arylene are optionally substituted with alkyl, hydroxyalkyl, haloalkyl, alkoxy, halogen, or hydroxyl.

In certain embodiments, R³ is H. In certain embodiments, R³ is (C₁-C₆)alkyl.

In certain embodiments, R⁴ is H. In certain embodiments, R⁴ is (C₁-C₆)alkyl. In certain embodiments, R⁴ is halogen. In certain embodiments, R⁴ is 5-12 membered heteroaryl, 5-12 membered heterocyclyl, or (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl. In certain embodiments, R⁴ is —C(O)NR³-heterocyclyl. In certain embodiments, R⁴ is 5-12 membered heteroaryl, optionally substituted with —N(R³)₂ or —OR³.

As described above, each R⁵ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁵ is H. In certain embodiments, R⁵ is (C₁-C₆)alkyl, wherein the alkyl is optionally substituted with —N(R³)₂ or —OR³. In certain embodiments, R⁵ is —C(O)OR³. In certain embodiments, R⁵ is —N(R³)₂.

As described above, each R⁶ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁶ is H. In certain embodiments, R⁶ is (C₁-C₆)alkyl, wherein the alkyl is optionally substituted with —N(R³)₂ or —OR³. In certain embodiments, R⁶ is —C(O)OR³. In certain embodiments, R⁶ is —N(R³)₂.

As described above, each R⁷ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁷ is H. In certain embodiments, R⁷ is (C₁-C₆)alkyl, wherein the alkyl is optionally substituted with —N(R³)₂ or —OR³. In certain embodiments, R⁷ is —C(O)OR³. In certain embodiments, R⁷ is —N(R³)₂.

As described above, each R⁸ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁸ is H. In certain embodiments, R⁸ is (C₁-C₆)alkyl, wherein the alkyl is optionally substituted with —N(R³)₂ or —OR³. In certain embodiments, R⁸ is —C(O)OR³. In certain embodiments, R⁸ is —N(R³)₂.

As described above, each Y is independently C(R³)₂ or a bond. In certain embodiments, Y is C(R³)₂. In certain embodiments, Y is CH₂. In certain embodiments, Y is a bond.

In certain embodiments, n is 1, 2, 3, 4, 5, 6, 7, or 8, or any range derivable therein. In certain embodiments, n is 1, 2, 3, or 4. In certain embodiments, n is 5, 6, 7, or 8. In certain embodiments, n is 9, 10, 11, or 12.

In certain embodiments, o is an integer from zero to 10, or any range derivable therein. In certain embodiments, o is 0, 1, 2, 3, 4, or 5. In certain embodiments, o is 6, 7, 8, 9, or 10. In certain embodiments, o is one to 7. In certain embodiments, o is one to 8. In certain embodiments, o is one to 9. In certain embodiments, o is 3 to 8.

In certain embodiments, o is an integer from zero to 30, or any range derivable therein. In certain embodiments, o is an integer from zero to 30, 29, 28, 27, or 26. In certain embodiments, o is an integer from zero to 25, 24, 23, 22, or 21. In certain embodiments, o is an integer from zero to 20, 19, 18, 17, or 16. In certain embodiments, o is an integer from zero to 15, 14, 13, 12, or 11.

In certain embodiments, p is 0, 1, 2, 3, 4, 5, or 6, or any range derivable therein. In certain embodiments, p is 7, 8, 9, 10, 11, or 12. In certain embodiments, p is 0, 1, 2, or 3. In certain embodiments, p is 4, 5, or 6.

In certain embodiments, q is an integer from zero to 10, or any range derivable therein. In certain embodiments, q is 0, 1, 2, 3, 4, or 5. In certain embodiments, q is 6, 7, 8, 9, or 10. In certain embodiments, q is one to 7. In certain embodiments, q is one to 8. In certain embodiments, q is one to 9. In certain embodiments, q is 3 to 8.

In certain embodiments, q is an integer from zero to 30, or any range derivable therein. In certain embodiments, q is an integer from zero to 30, 29, 28, 27, or 26. In certain embodiments, q is an integer from zero to 25, 24, 23, 22, or 21. In certain embodiments, q is an integer from zero to 20, 19, 18, 17, or 16. In certain embodiments, q is an integer from zero to 15, 14, 13, 12, or 11.

As described above, r is an integer from one to 6. In certain embodiments, r is one. In certain embodiments, r is 2. In certain embodiments, r is 3. In certain embodiments, r is 4. In certain embodiments, r is 5. In certain embodiments, r is 6.

As described above, when R²⁸ and R⁴⁰ are H, then R³² is not ═O. In certain embodiments, the compound is not rapamycin, as shown below:

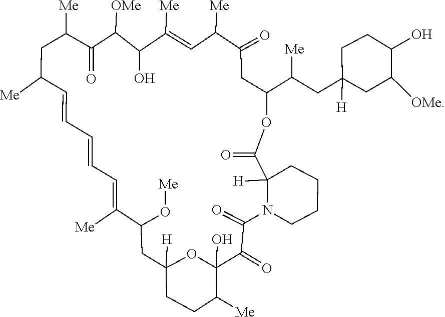 [SMILES: COC1CC2CCC(C)C(O)(O2)C(=O)C(=O)N2CCCCC2C(=O)OC(C(C)CC2CCC(O)C(OC)C2)CC(=O)C(C)/C=C(\C)C(O)C(OC)C(=O)C(C)CC(C)/C=C/C=C/C=C/1C]

In certain embodiments, in Formula Ia or Ic. R³² is —O—C(═Z¹)—R^{32a}. In certain embodiments, R³² is —O—C(═Z¹)—R^{32a}; wherein R^{32a} is -A¹-L¹-A²-B; -A¹-A²-B; or -L²-A¹-L¹-A²-L³-B. In certain embodiments, in Formula Ia or Ic, R²⁸ is —C(═Z¹)—R^{28a}. In certain embodiments, R²⁸ is —C(═Z¹)—R^{28a}; wherein R^{28a} is -A¹-L¹-A²-B; -A¹-A²-B; or -L²-A¹-L¹-A²-L³-B. In certain embodiments, in Formula Ia or Ic, R⁴⁰ is —C(═Z¹)—R^{40a}. In certain embodiments, R⁴⁰ is —C(═Z¹)—R^{40a}, wherein R^{40a} is -A¹-L¹-A²-B; -A¹-A²-B; or -L²-A¹-L¹-A²-L³-B.

The present disclosure provides a compound of Formula Ia or Ic, or a pharmaceutically acceptable salt or tautomer thereof, having one, two, or three of the following features:

a) R³² is —O—C(═Z¹)—R^{32a};

b) R²⁸ is —C(═Z¹)—R^{28a};

c) R⁴⁰ is —C(═Z¹)—R^{40a}.

The present disclosure provides a compound of Formula Ia or Ic, or a pharmaceutically acceptable salt or tautomer thereof, having one, two, or three of the following features:

a) R¹² is —O—C(═Z¹)—R^{32a}; wherein R^{32a} is -A¹-L¹-A²-B; -A¹-A²-B; or -L²-A¹-L¹-A²-L³-B;

b) R²⁸ is —C(═Z¹)—R^{28a}; wherein R^{28a} is -A¹-L¹-A²-B; -A¹-A²-B; or -L²-A¹-L¹-A²-L³-B;

c) R⁴⁰ is —C(═Z¹)—R^{40a}, wherein R^{40a} is -A¹-L¹-A²-B; -A¹-A²-B; or -L²-A¹-L¹-A²-L³-B.

The present disclosure provides a compound of Formula Ia or Ic, or a pharmaceutically acceptable salt or tautomer thereof, having one, two, or three of the following features:

a) R⁴⁰ is —C(═Z¹)—R^{40a};

b) R^{40a} is -A¹-L¹-A²-B; -A¹-A²-B; -L²-A¹-L¹-A²-L³-B;

c) R³² is —OR³, such as —OH.

The present disclosure provides a compound of Formula Ia or Ic, or a pharmaceutically acceptable salt or tautomer thereof, having one, two, three, or four of the following features:

a) one of R^{28a}, R^{32a}, and R^{40a} is -A¹-L¹-A²-B;

b) A1 is absent;

c) A² is absent;

d) L¹ is

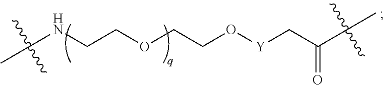

e) B is

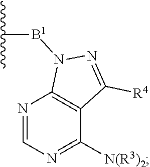

f) B¹ is

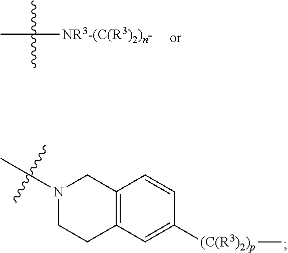

g) R⁴ is 5-12 membered heteroaryl, optionally substituted with —N(R³)₂ or —OR³.

The resent disclosure provides a compound of formula:

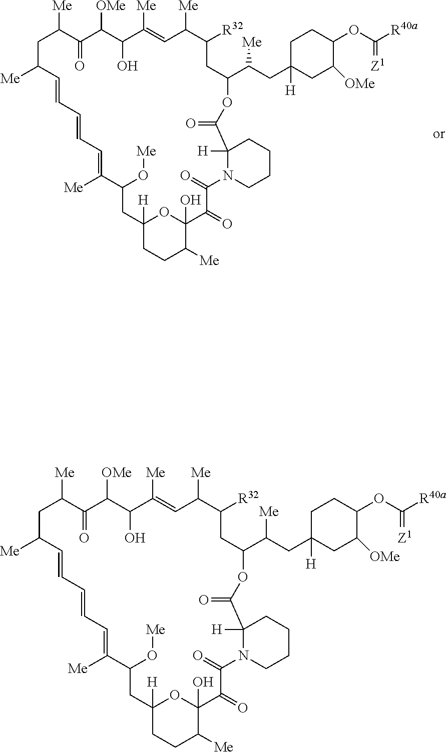

or a pharmaceutically acceptable salt or tautomer thereof, having one, two, three, or four of the following features:

a) Z¹ is O;

b) A¹ is absent:

c) L¹ is

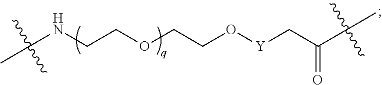

d) B is

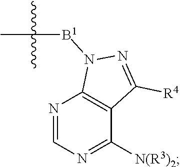

e) B¹ is

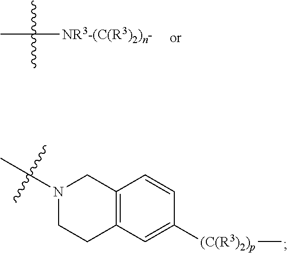

f) R⁴ is 5-12 membered heteroaryl, optionally substituted with —N(R³)₂ or —OR³; and

g) R³² is ═O.

In the above, R^{40a} can be -A¹-L¹-A²-B; -A¹-A²-B; or -L²-A¹-L¹-A²-L³-B.

The resent disclosure provides a compound of formula:

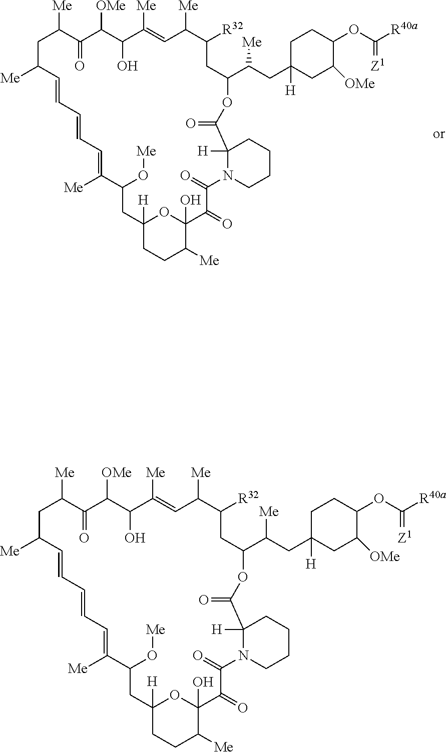

or a pharmaceutically acceptable salt or tautomer thereof, having one, two, three, or four of the following features:

a) Z¹ is O;

b) A¹ is absent;

c) L¹ is

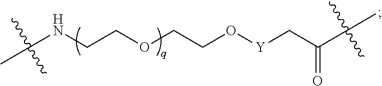

d) B is

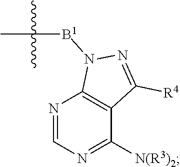

### e) B¹ is

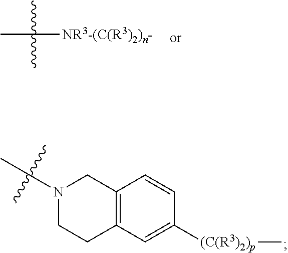

f) R⁴ is 5-12 membered heteroaryl, optionally substituted with —N(R³)₂ or —OR³; and

g) R³² is —OH.

In the above, R^{40a} can be -A¹-L¹-A²-B; -A¹-A²-B; or -L²-A¹-L²-A²-L³-B.

The present disclosure provides a compound of formula:

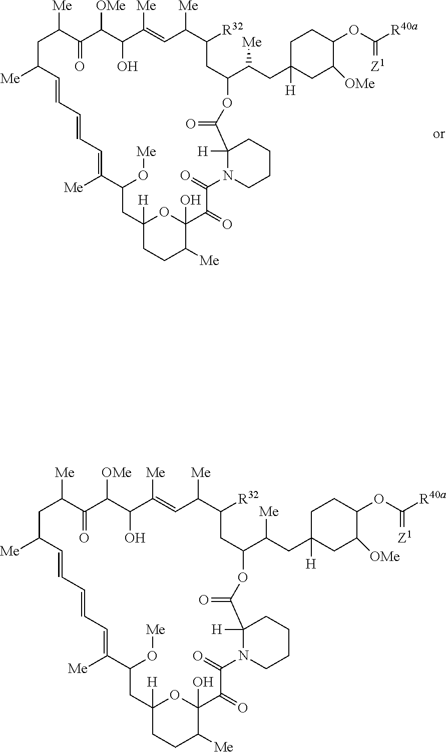

or a pharmaceutically acceptable salt or tautomer thereof, having one, two, three, or four of the following features:

a) Z¹ is O;

b) A¹ is

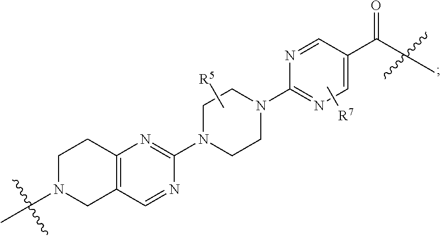

c) L¹ is

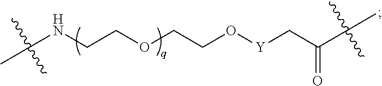

### d) B is

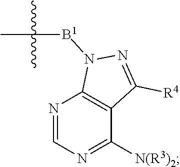

e) B¹ is

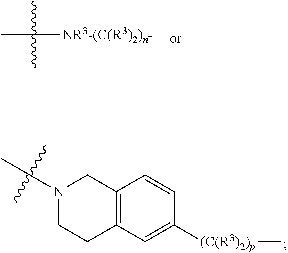

f) R⁴ is 5-12 membered heteroaryl, optionally substituted with —N(R³)₂ or —OR³; and

g) R³² is ═O.

In the above, R^{40a} can be -A¹-L¹-A²-B; -A¹-A²-B; or -L²-A¹-L¹-A²-L³-B.

The present disclosure provides a compound of formula:

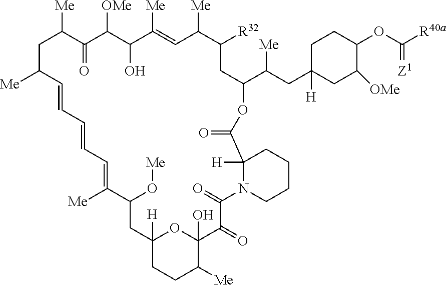

or a pharmaceutically acceptable salt or tautomer thereof, having one, two, three, or four of the following features:

a) Z¹ is O;

b) A¹ is absent;

c) L¹ is

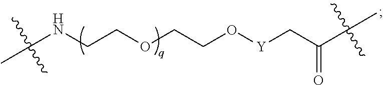

d) B is

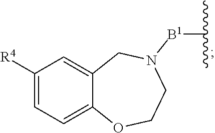

### e) B¹ is

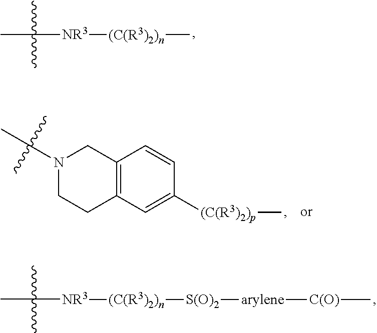

wherein the arylene is optionally substituted with alkyl, hydroxyalkyl, haloalkyl, alkoxy, halogen, or hydroxyl;

f) R⁴ is 5-12 membered heteroaryl, optionally substituted with —N(R³)₂ or —OR³; and

g) R³² is —OH.

In the above, R^{40a} can be -A¹-L¹-A²-B; -A¹-A²-B; or -L²-A¹-L¹-A²-L³-B.

The present disclosure provides a compound of formula:

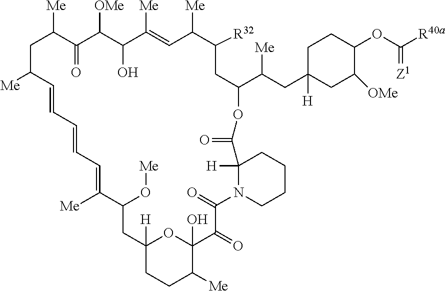

or a pharmaceutically acceptable salt or tautomer thereof, having one, two, three, or four of the following features:

a) Z¹ is O;

b) A¹ is

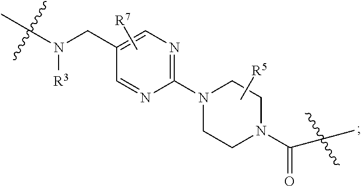

c) A² is

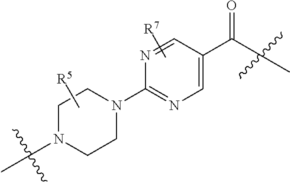

d) L¹ is

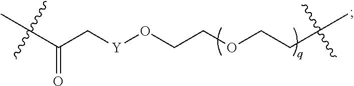

### e) B is

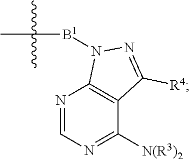

f) B¹ is

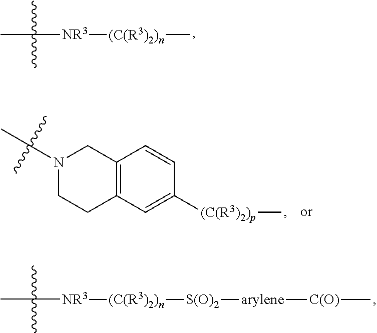

wherein the arylene is optionally substituted with alkyl, hydroxyalkyl, haloalkyl, alkoxy, halogen, or hydroxyl;

g) R⁴ is 5-12 membered heteroaryl, optionally substituted with —N(R³)₂ or —OR³; and

h) R³² is —OH.

In the above, R^{40a} can be -A¹-L¹-A²-B; -A¹-A²-B; or -L²-A¹-L¹-A²-L³-B.

In certain embodiments, in Formula Ia or Ic, R^{40a} is any organic moiety, which may have a molecular weight (e.g. the sum of the atomic masses of the atoms of the substituent) of less than 15 g/mol, 50 g/mol, 100 g/mol, 150 g/mol, 200 g/mol, 250 g/mol, 300 g/mol, 350 g/mol, 400 g/mol, 450 g/mol, or 500 g/mol.

In certain embodiments, the present disclosure provides for a compound selected from below or a pharmaceutically acceptable salt or tautomer thereof.

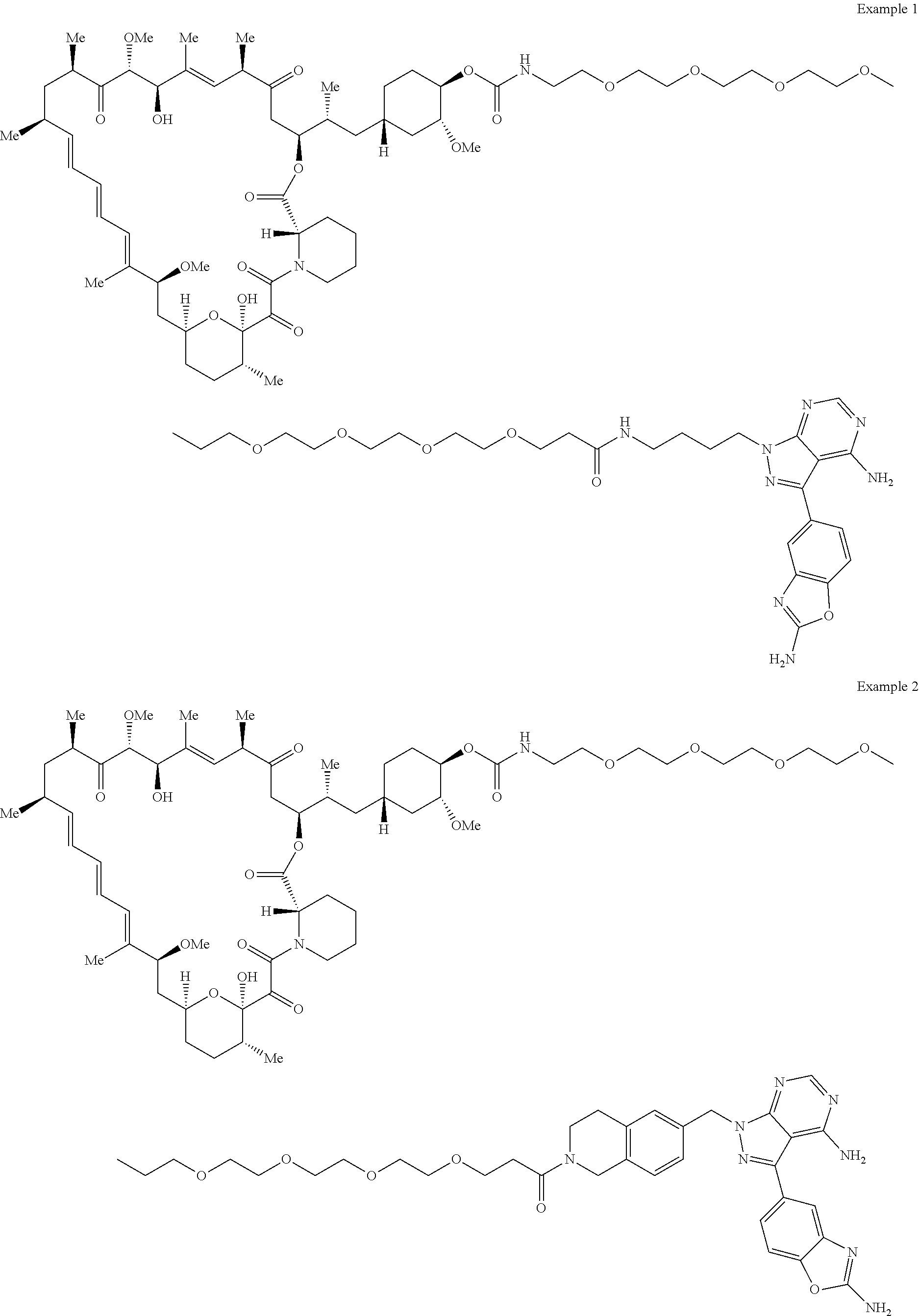

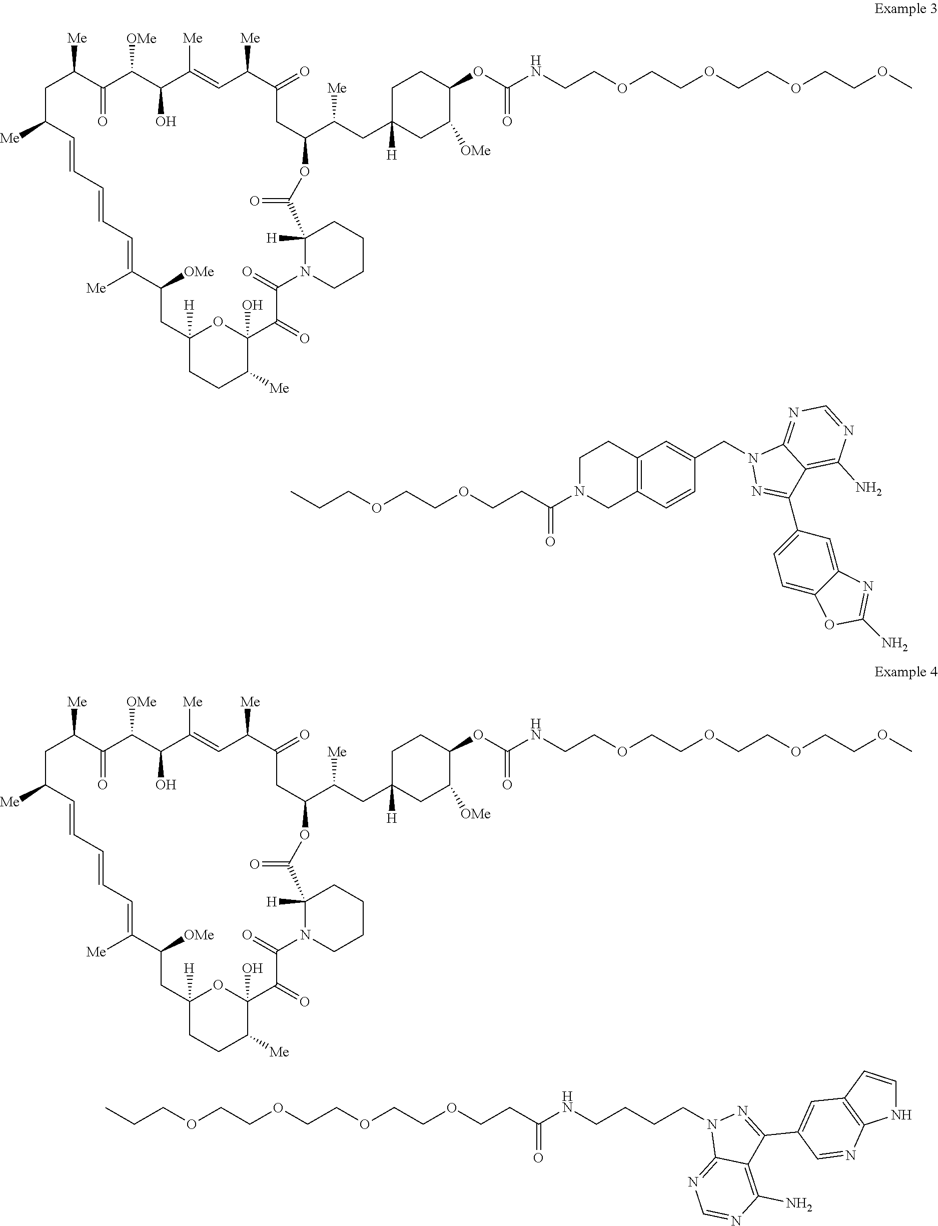

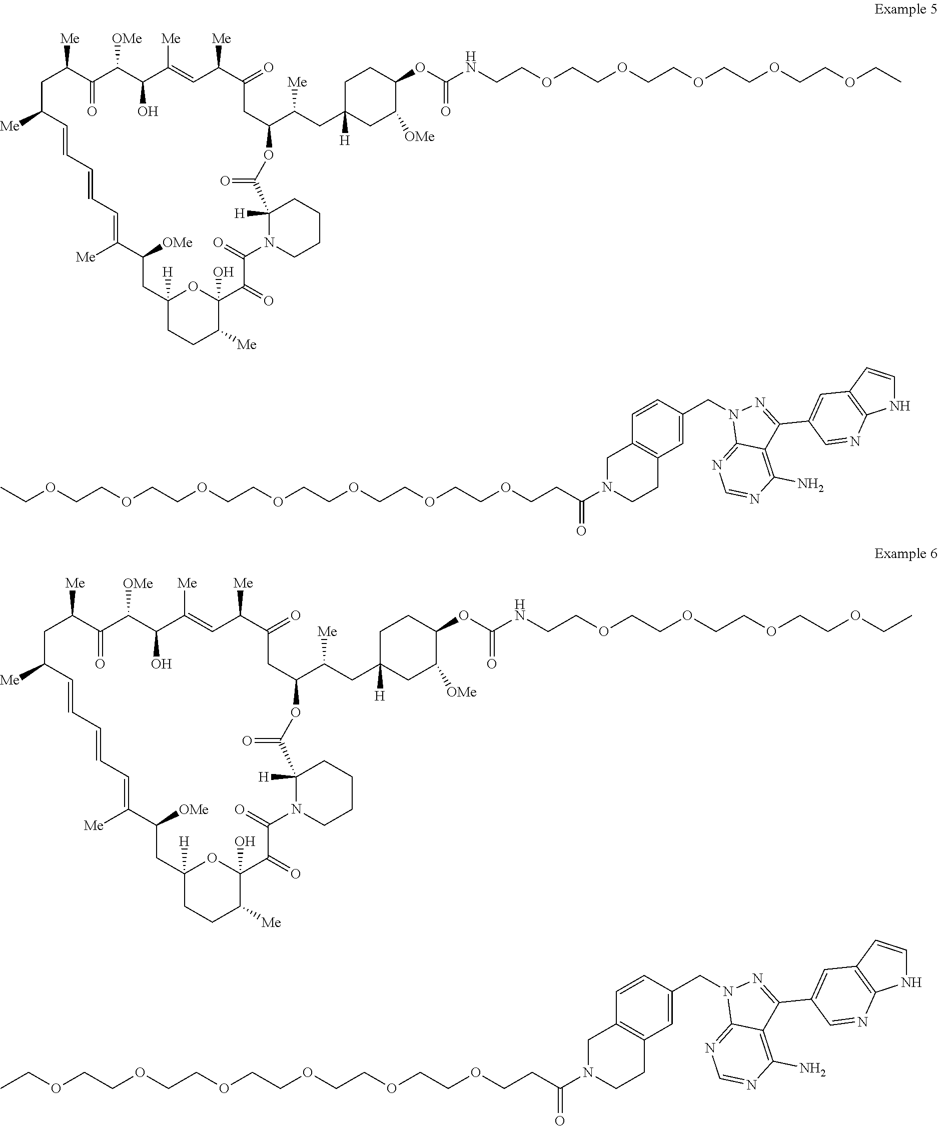

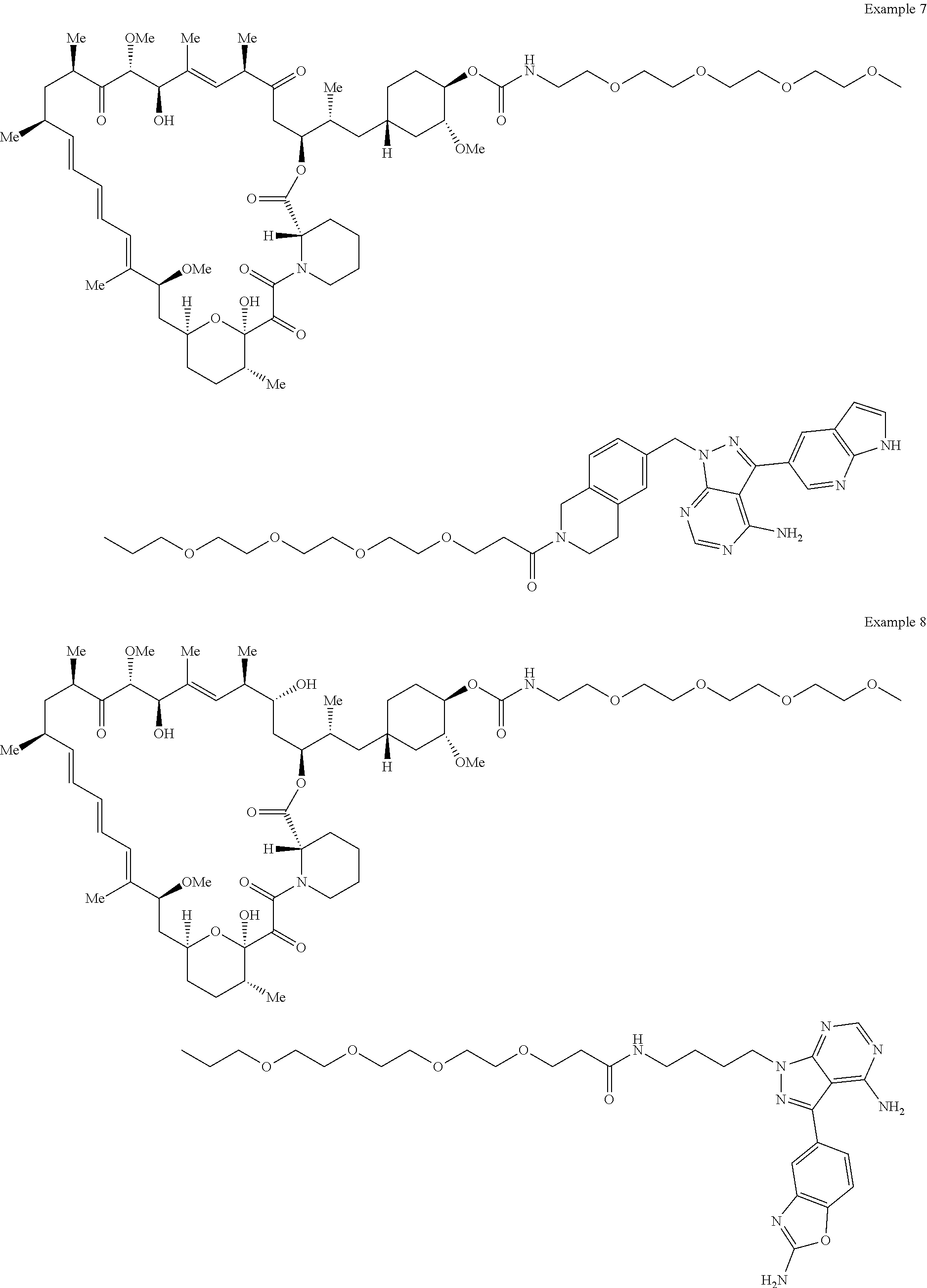

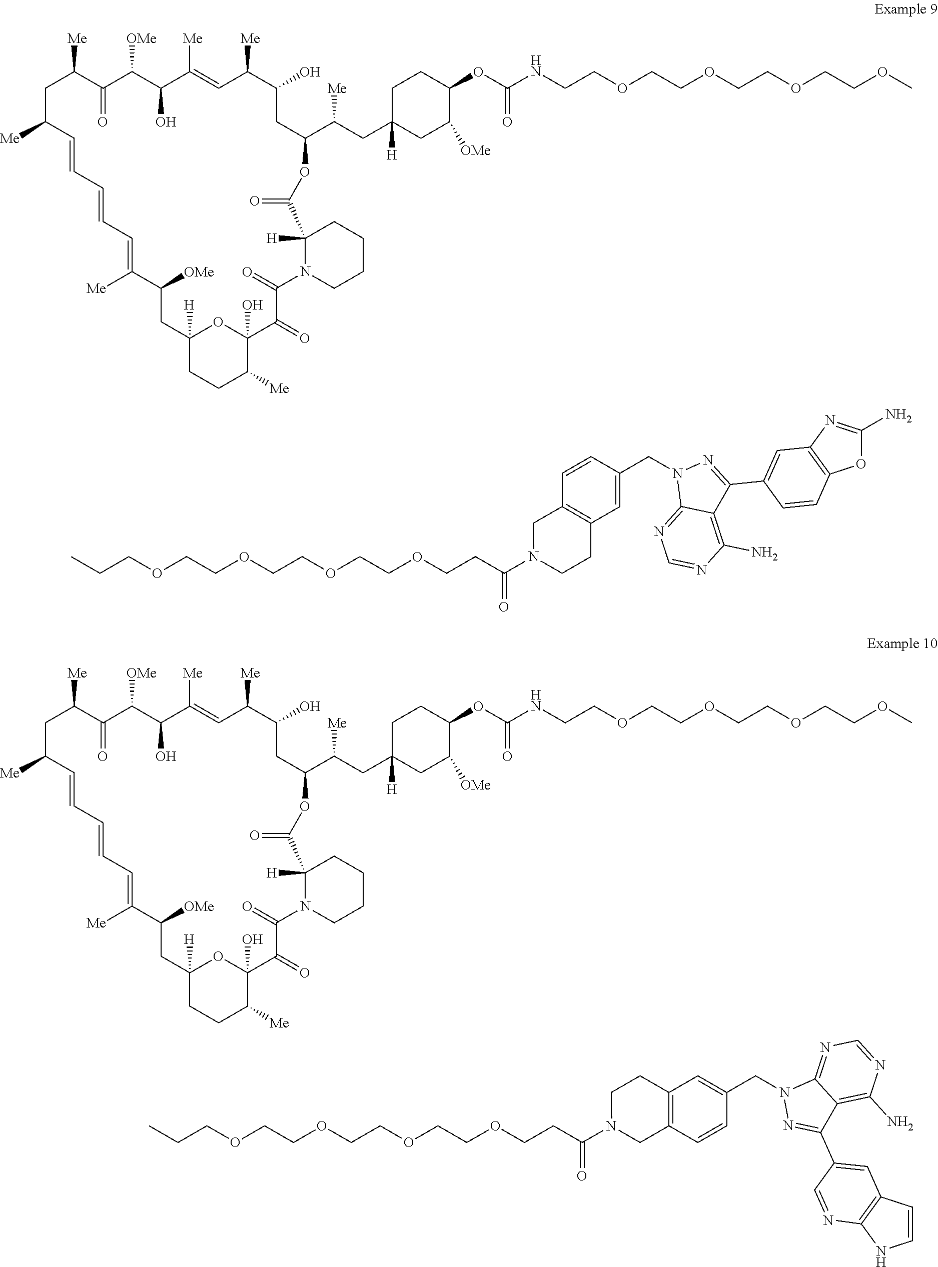

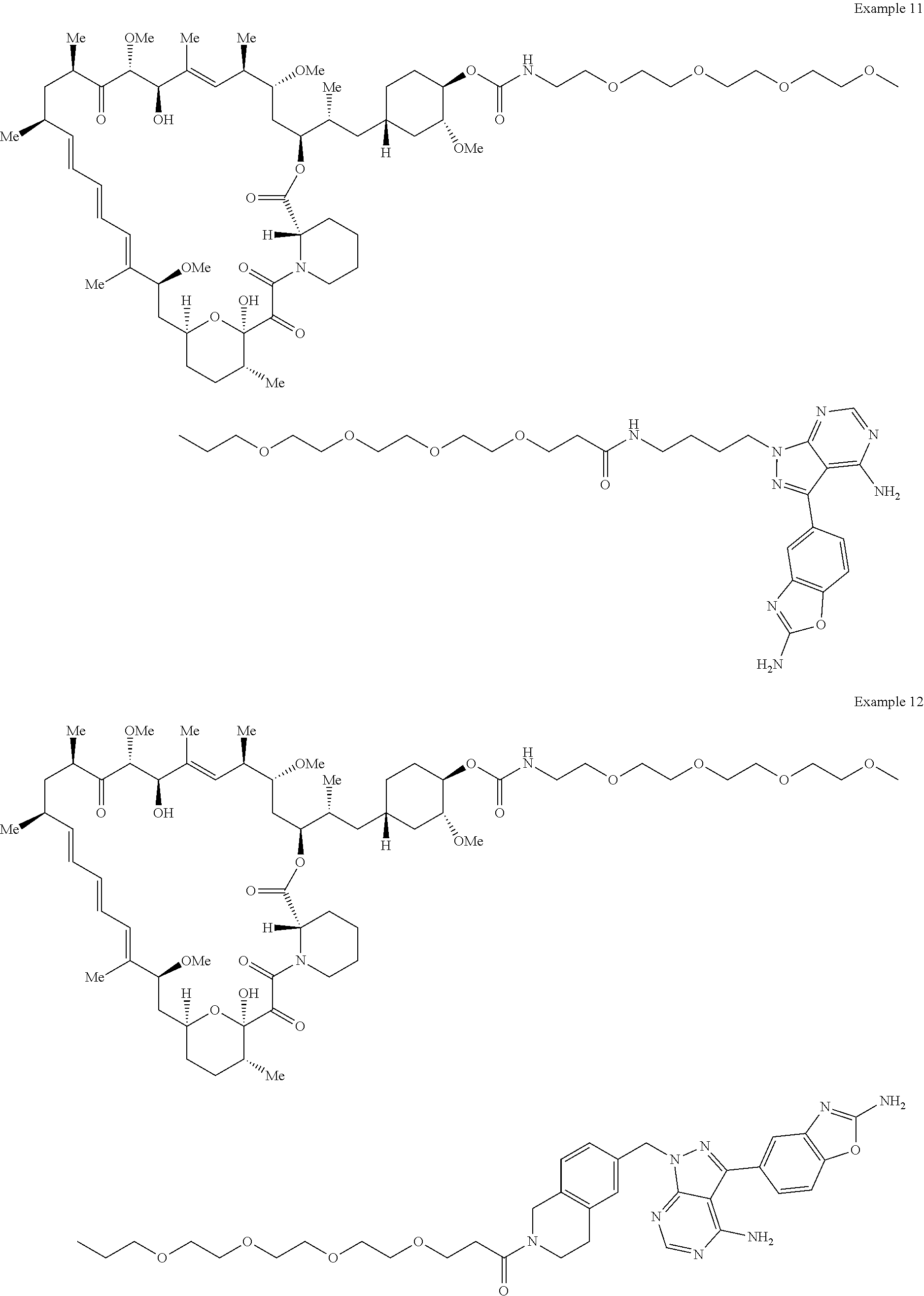

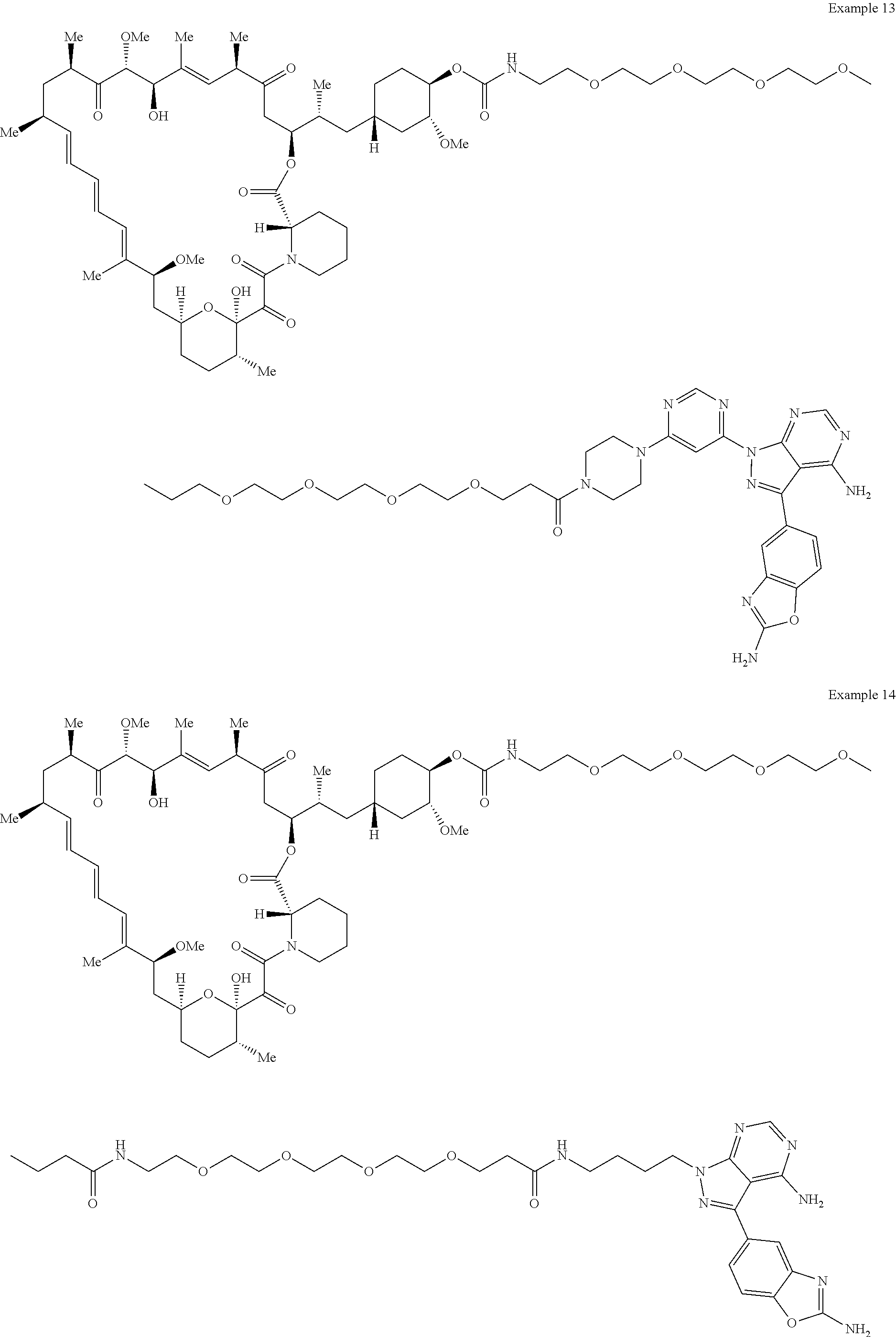

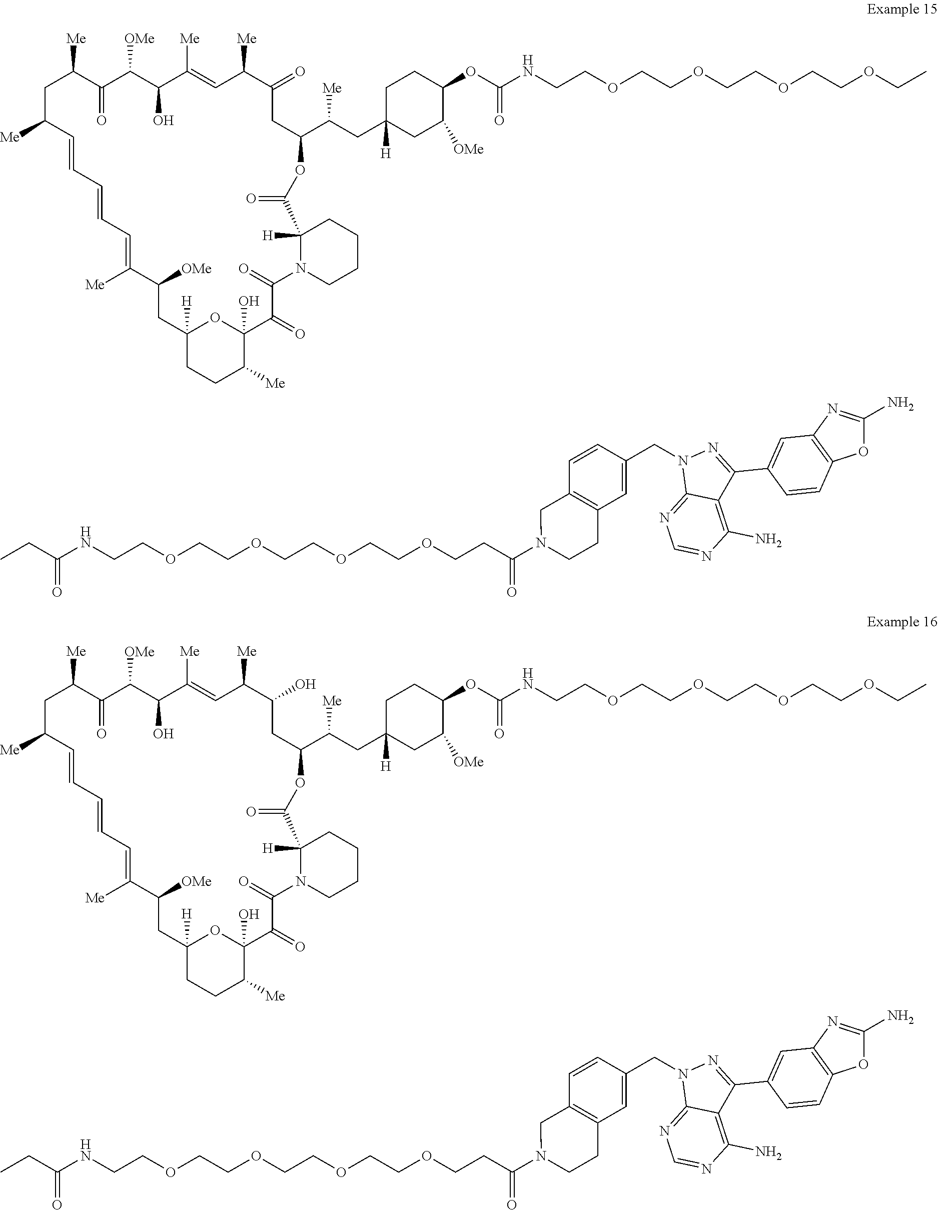

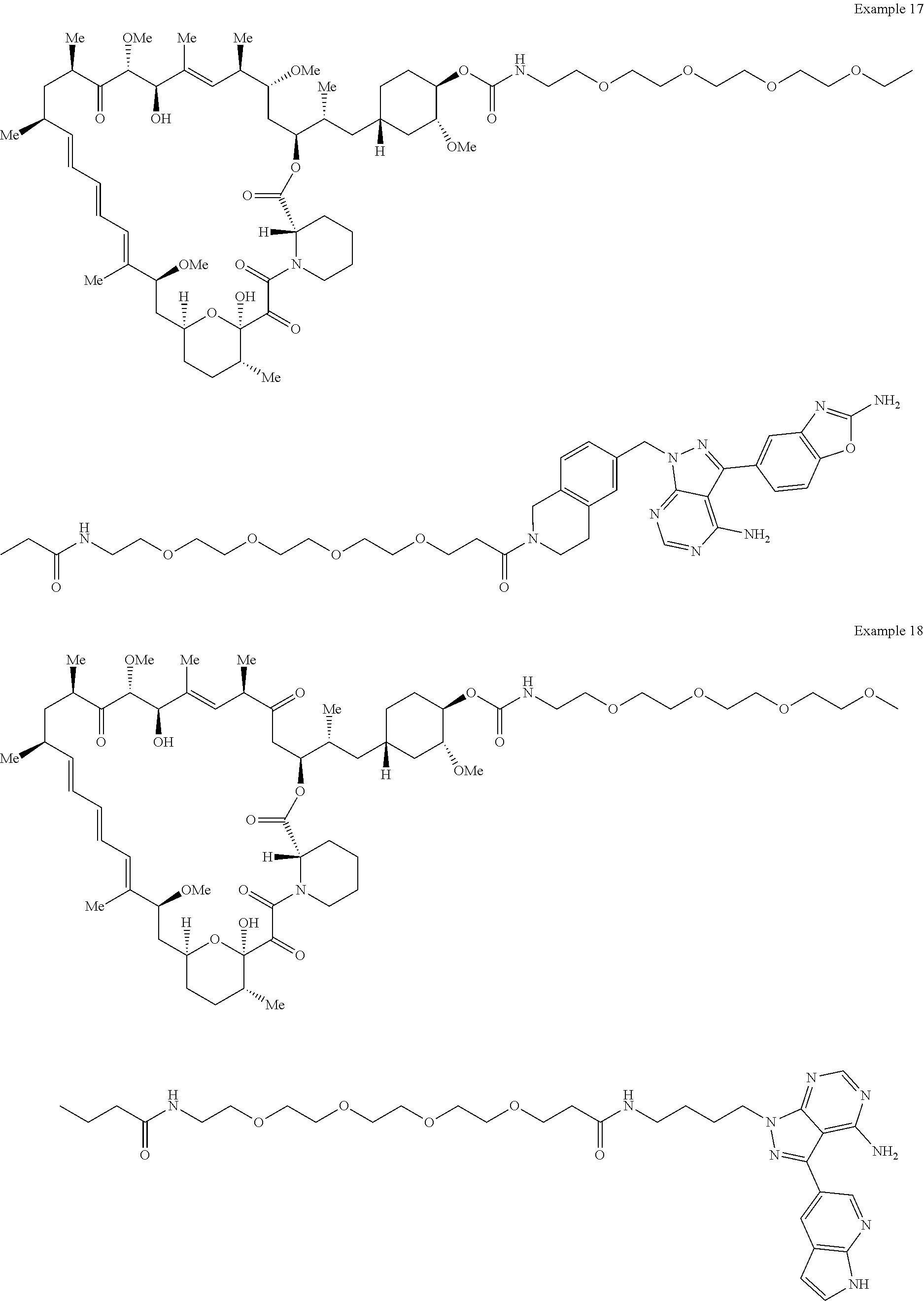

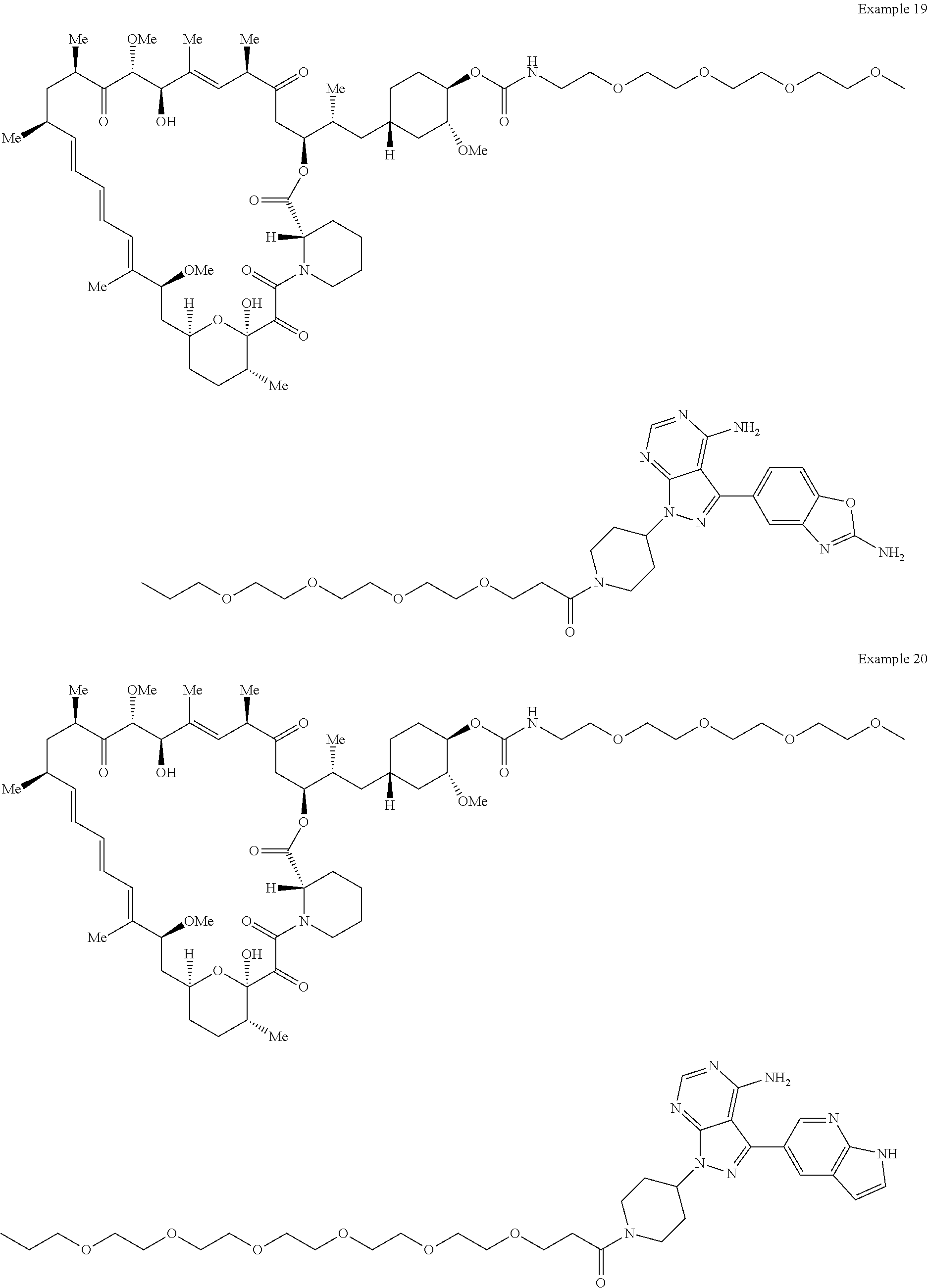

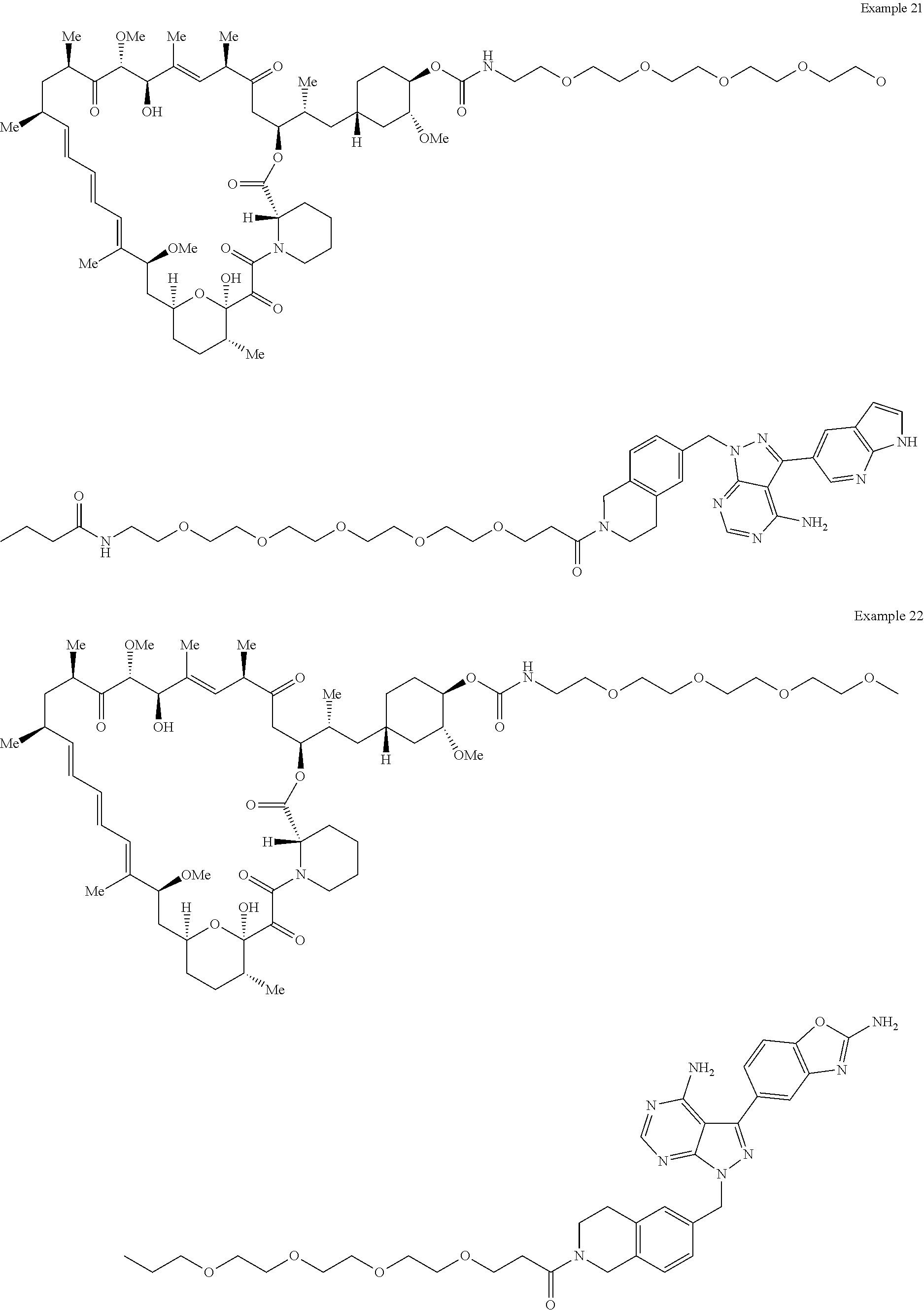

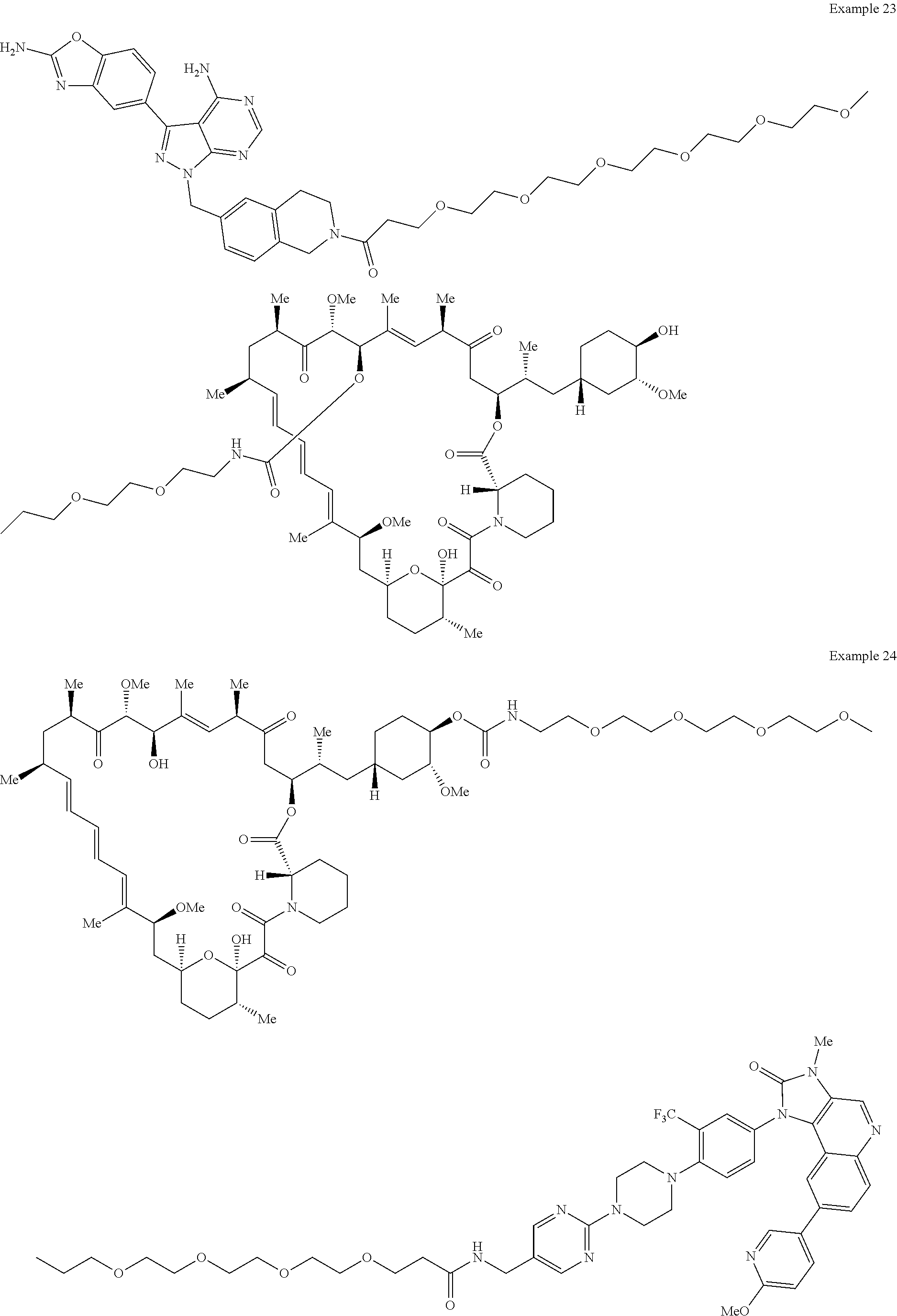

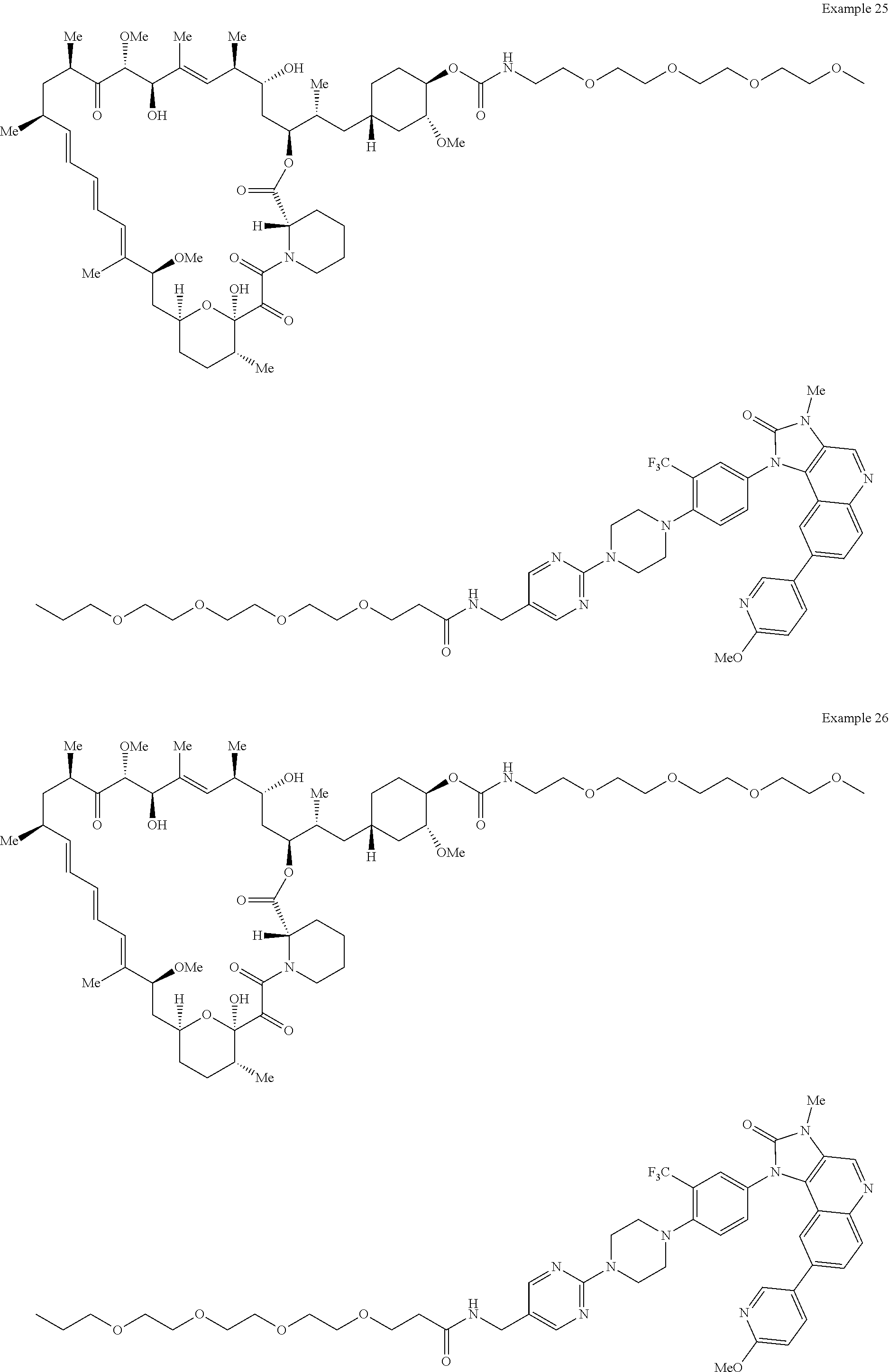

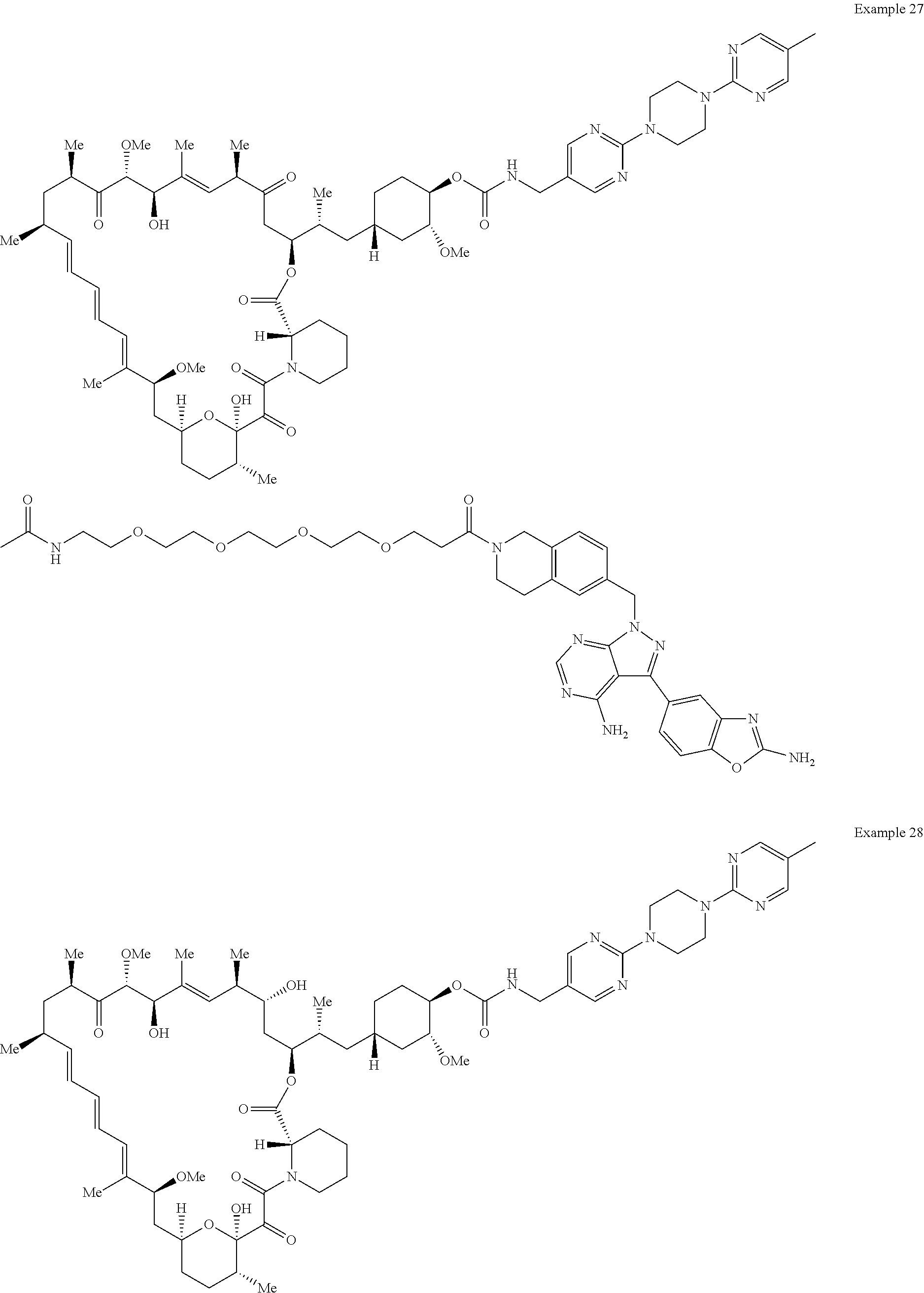

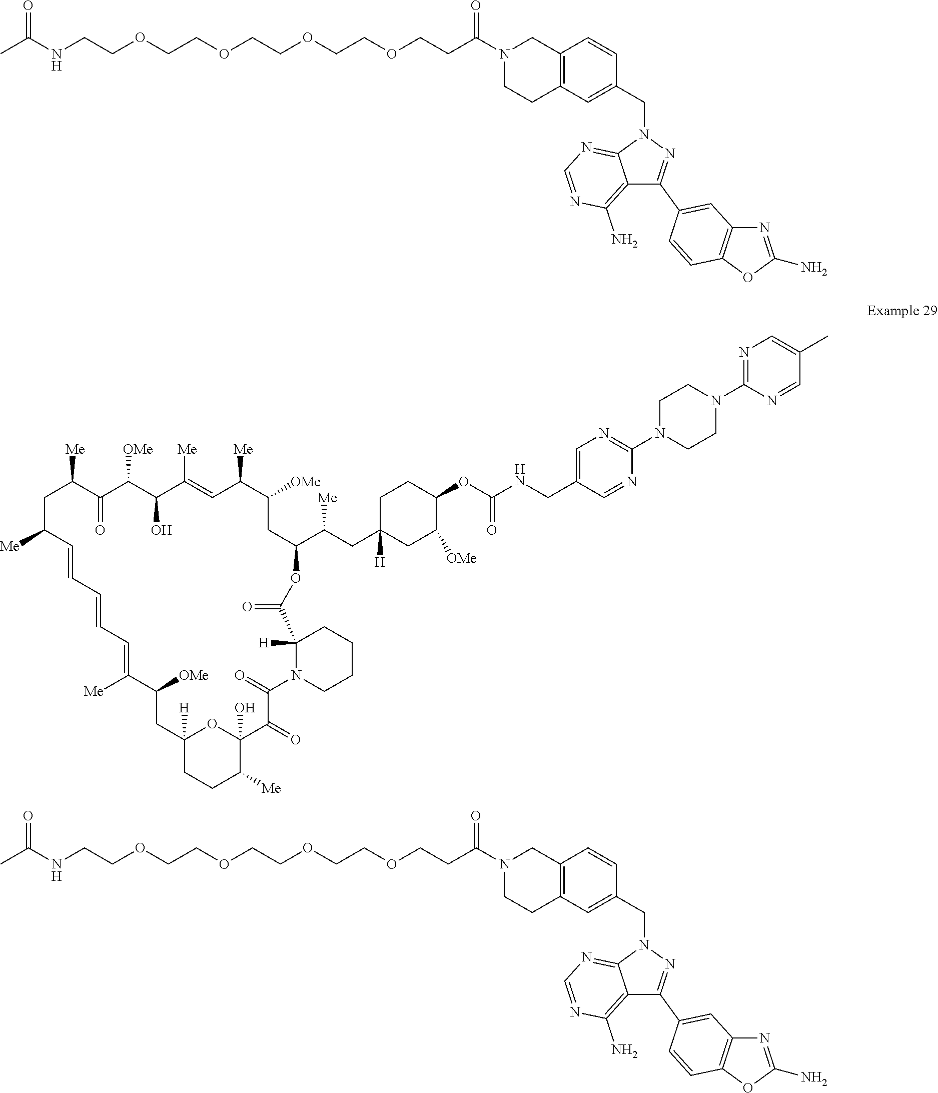

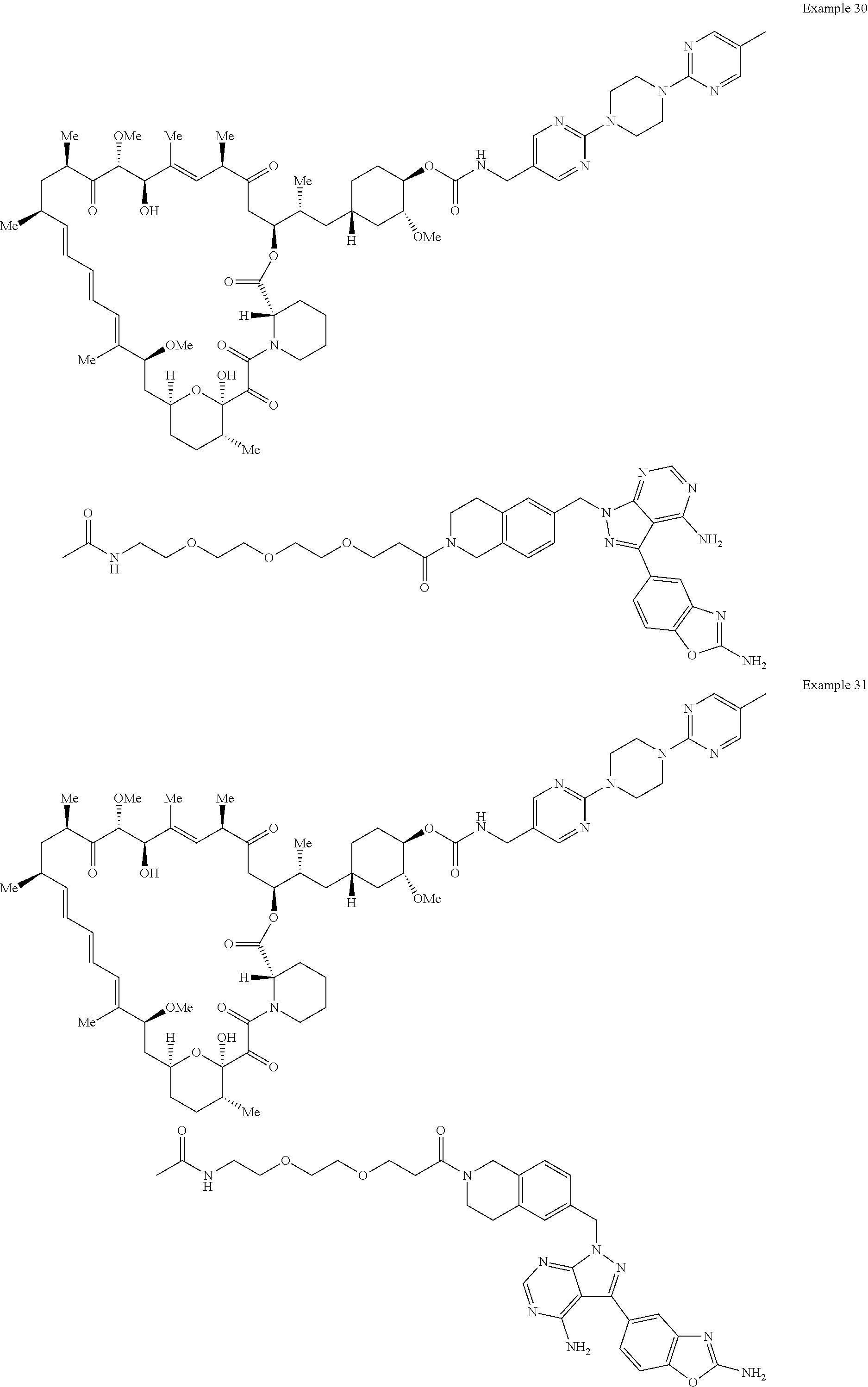

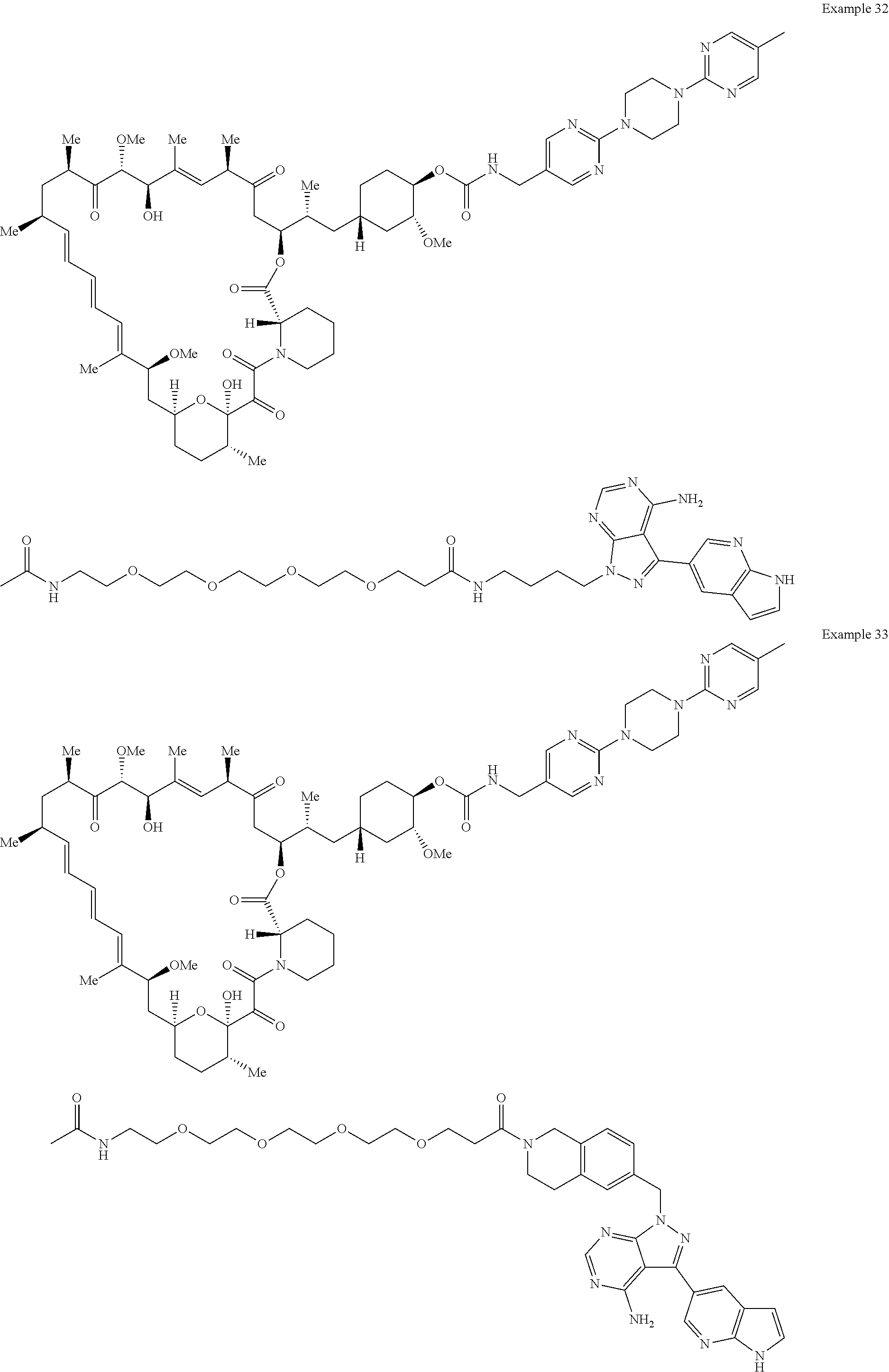

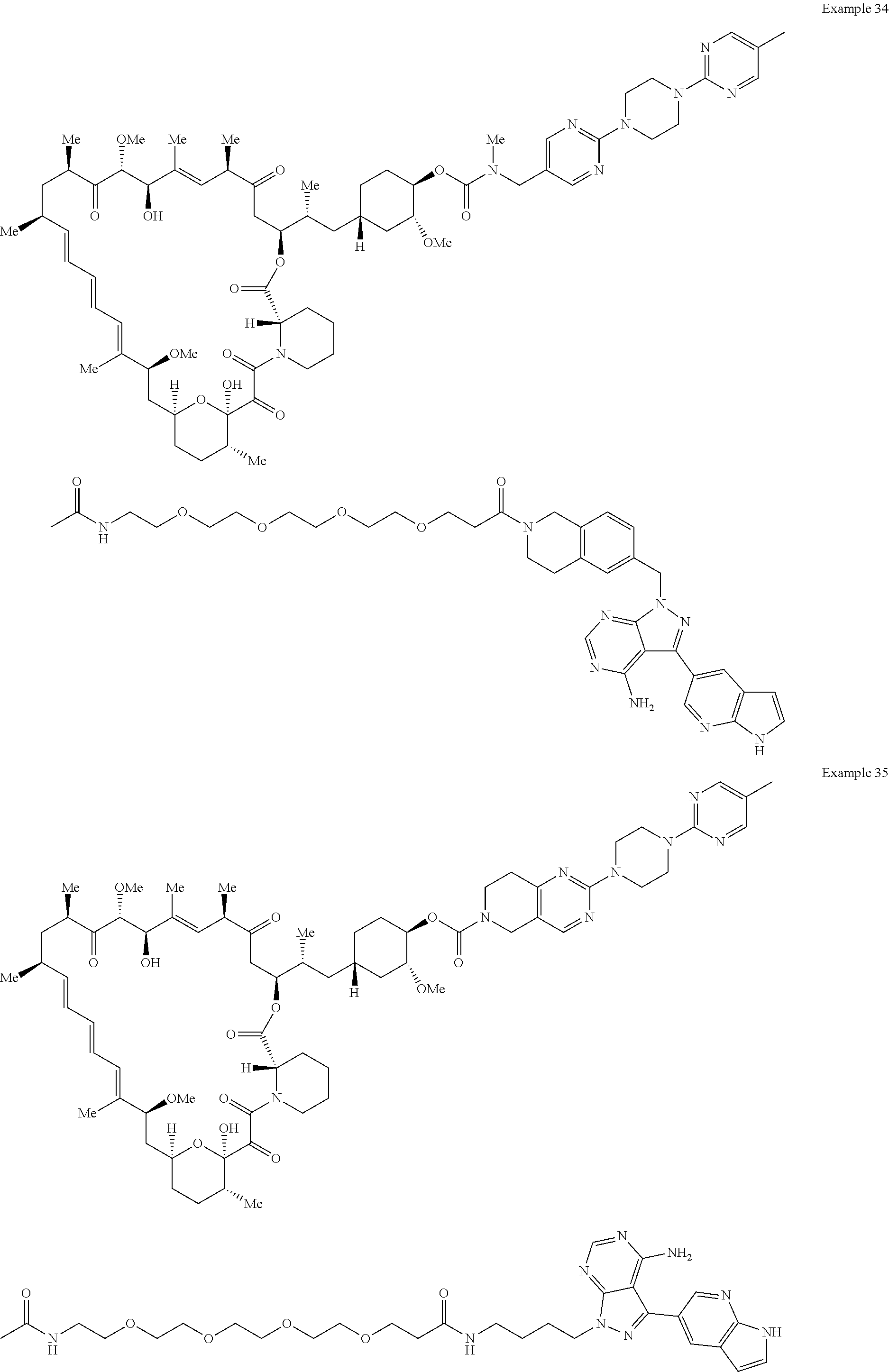

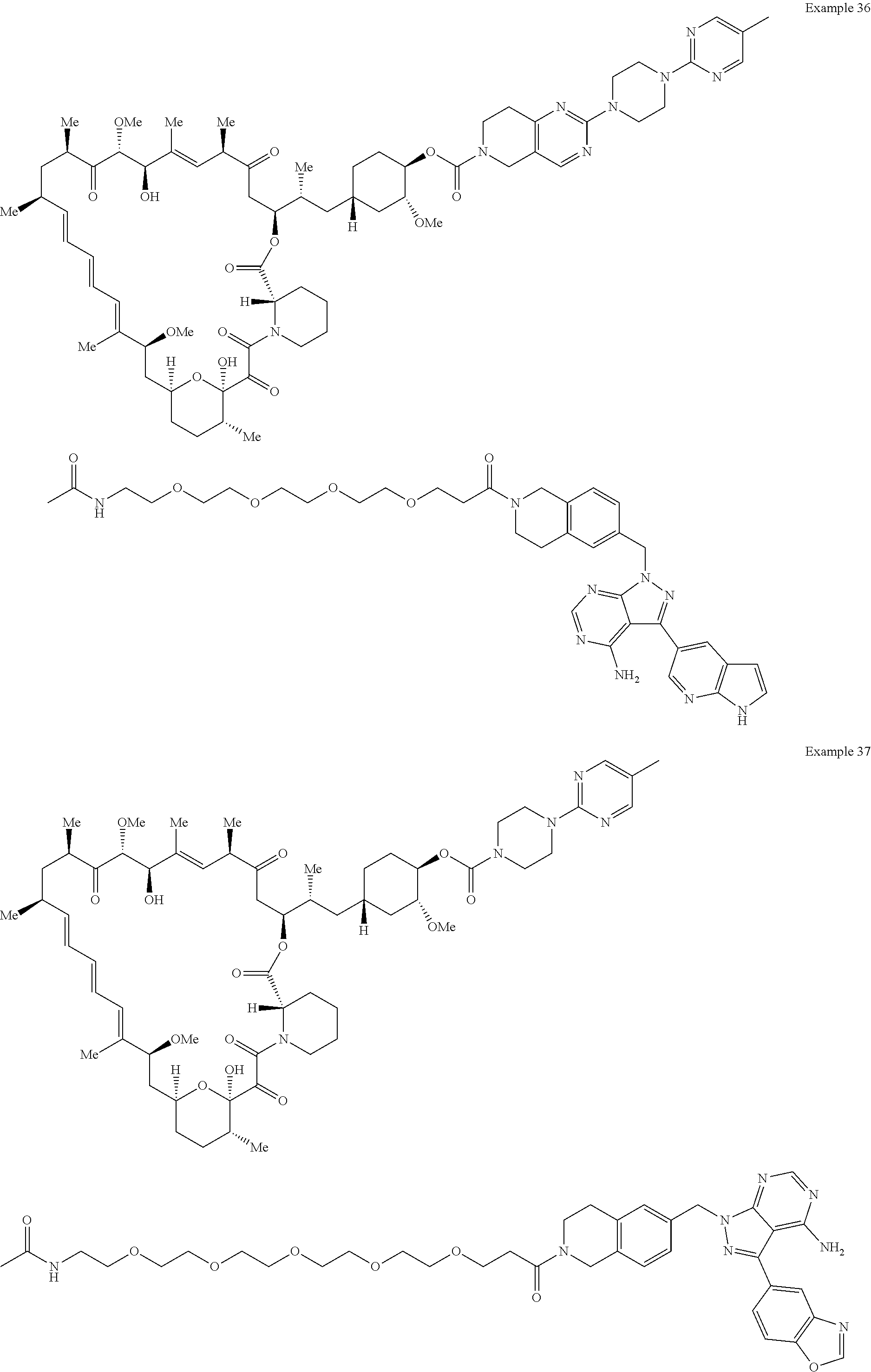

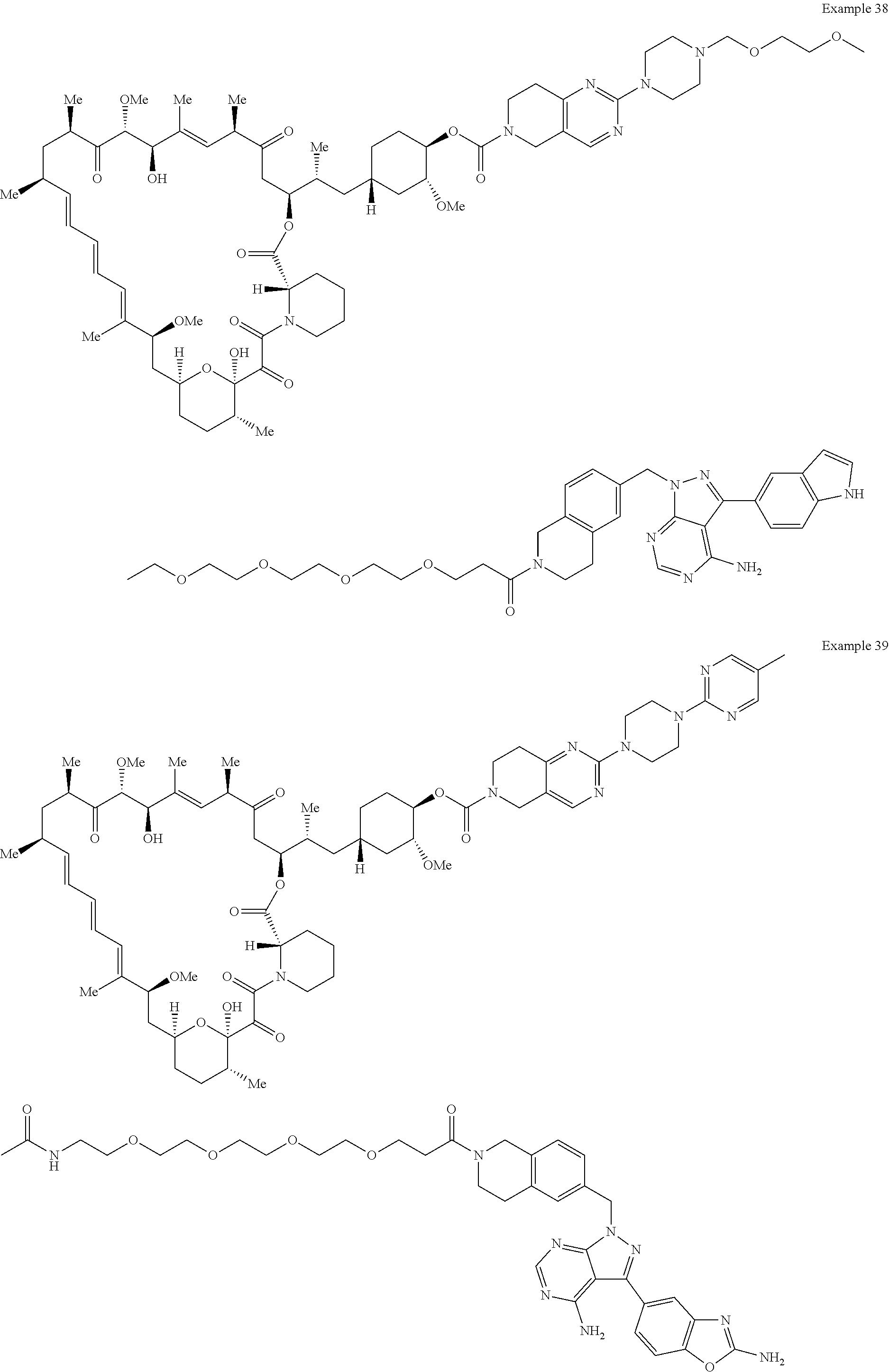

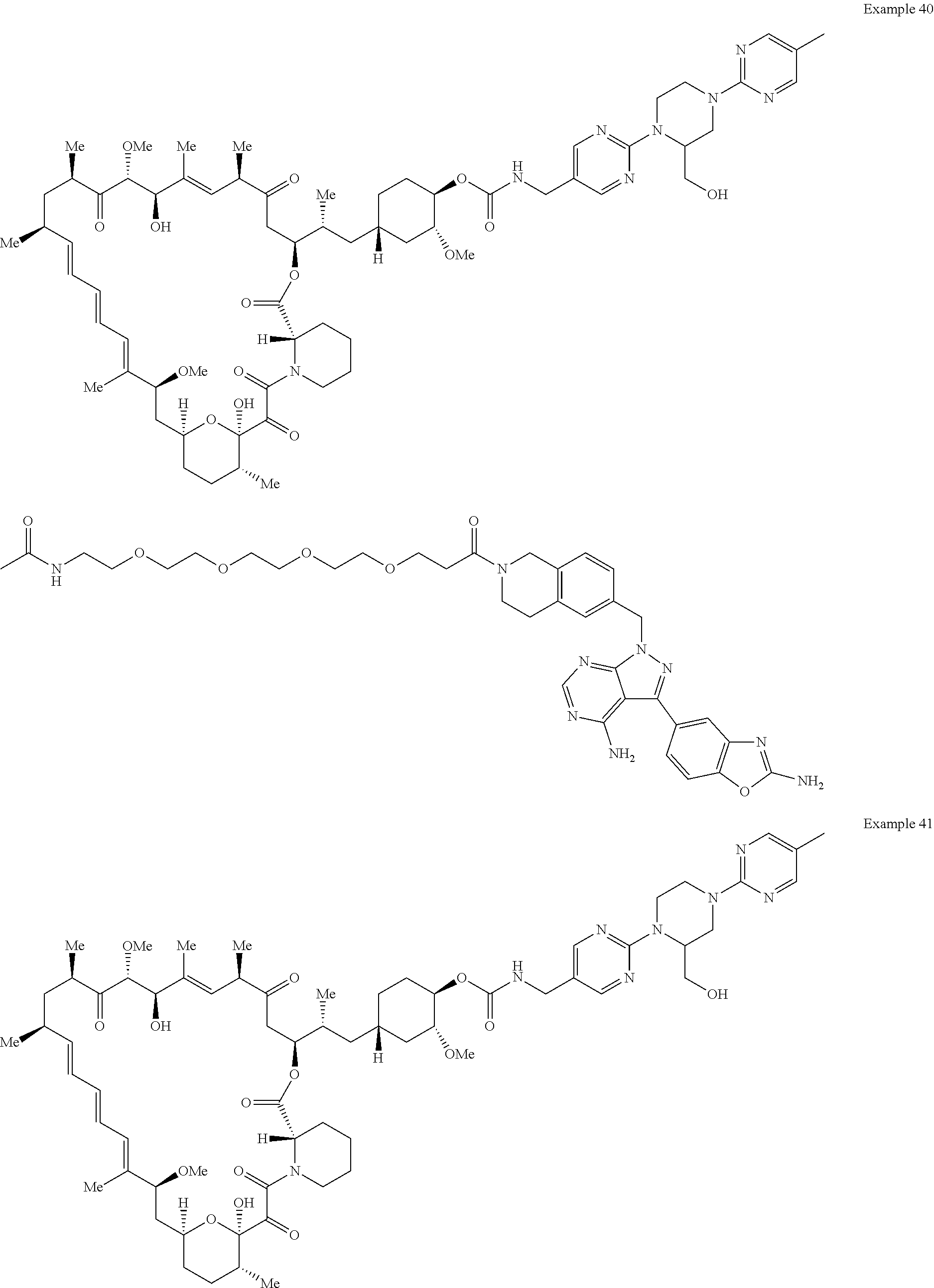

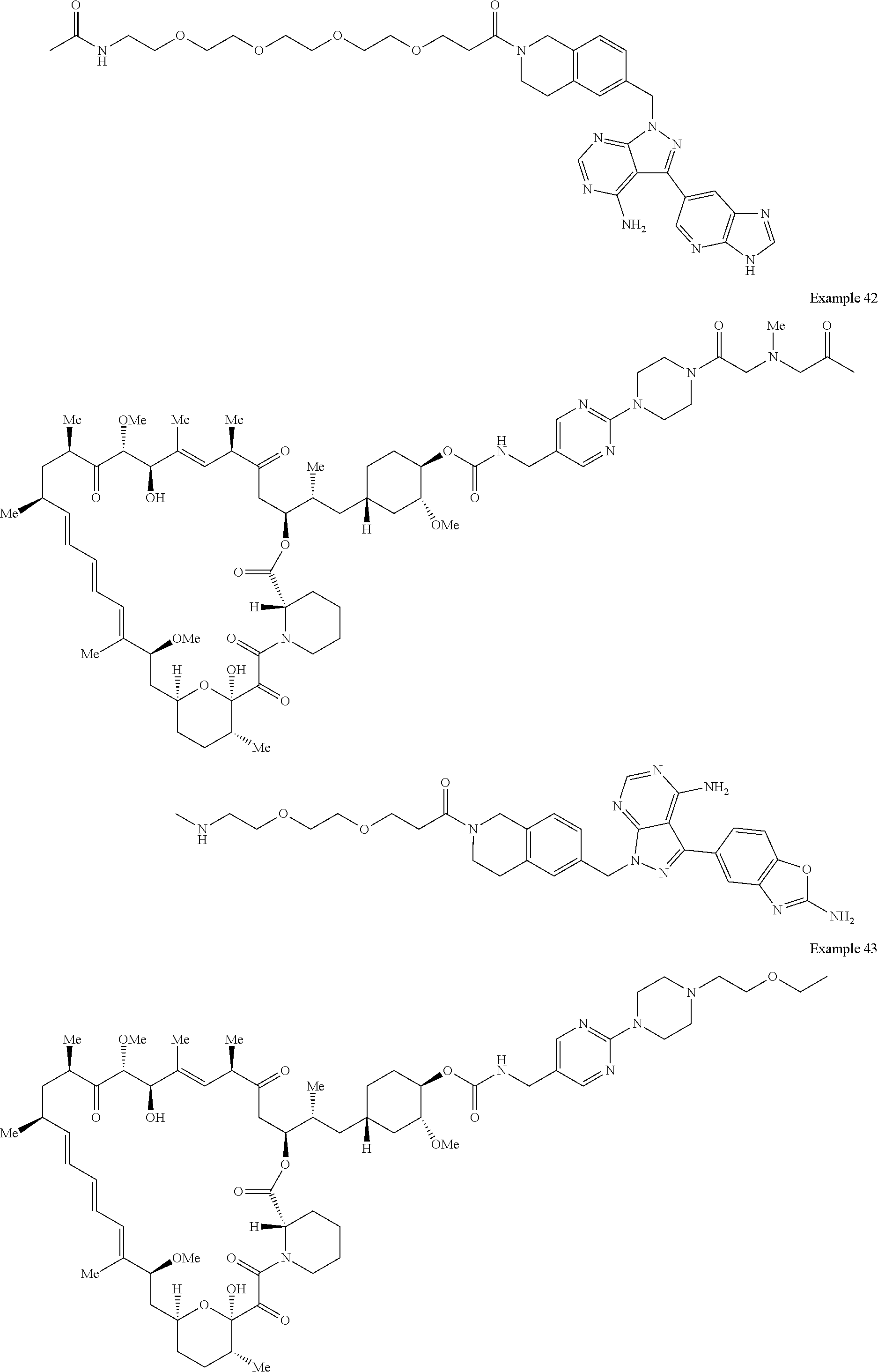

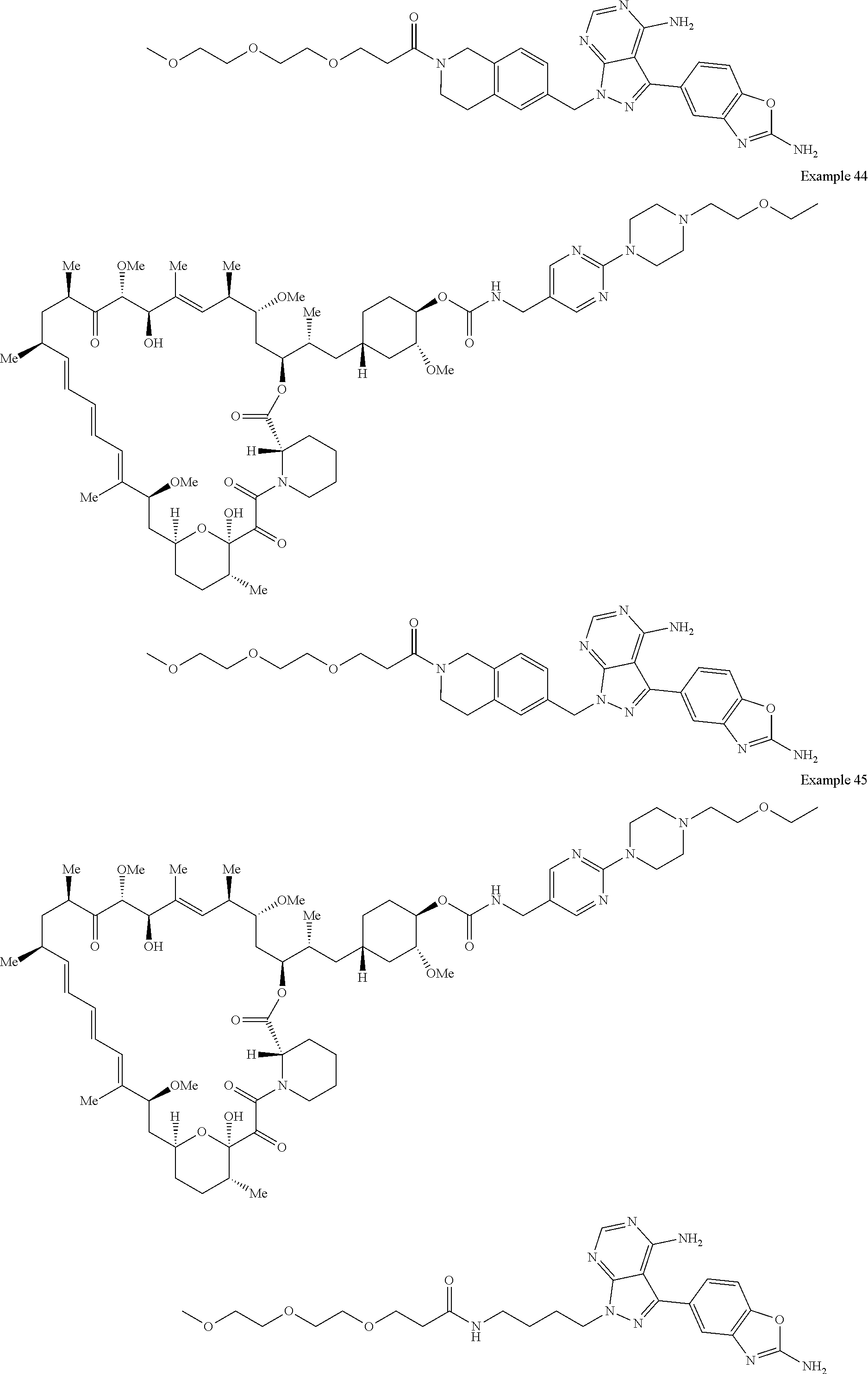

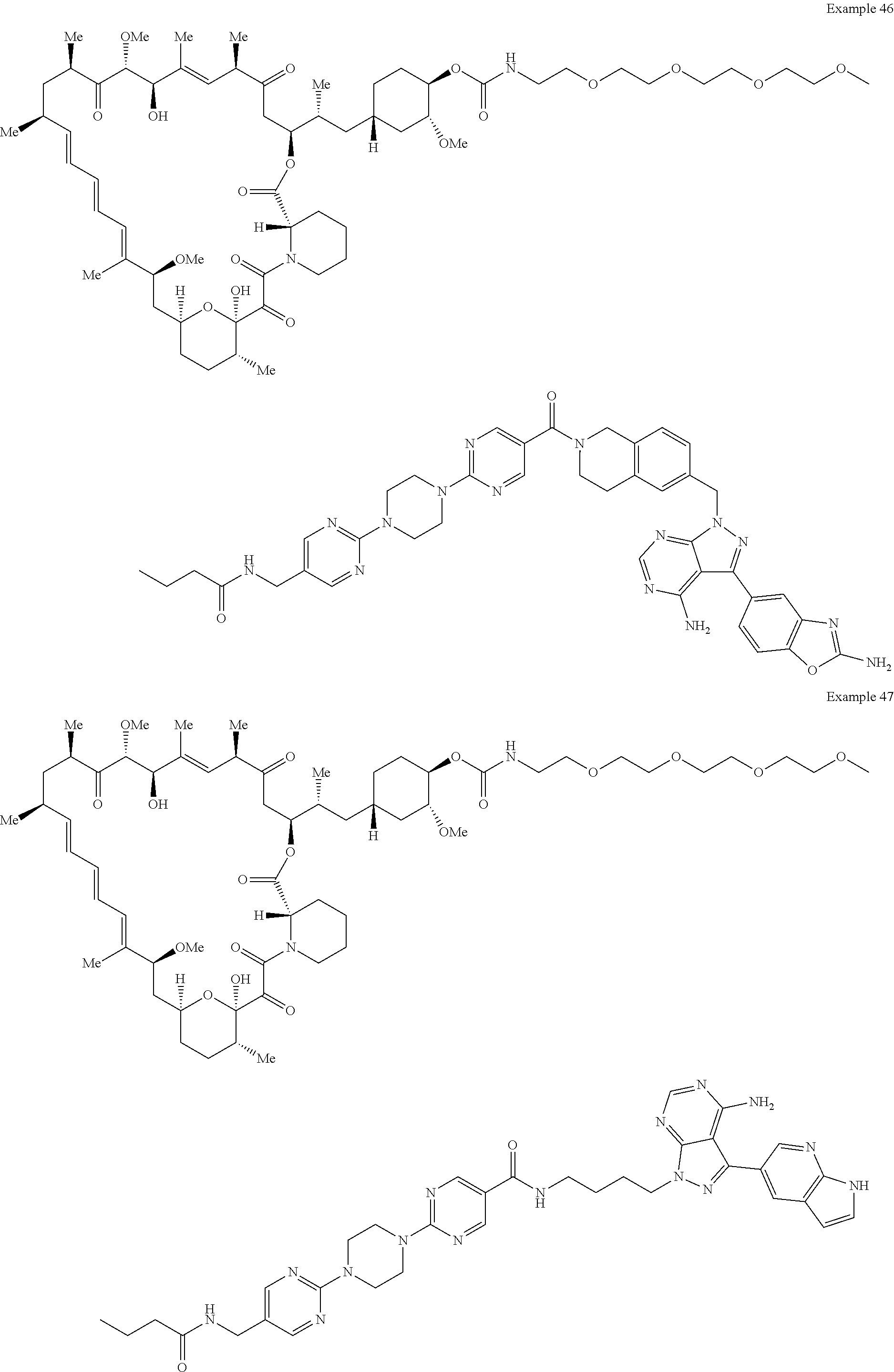

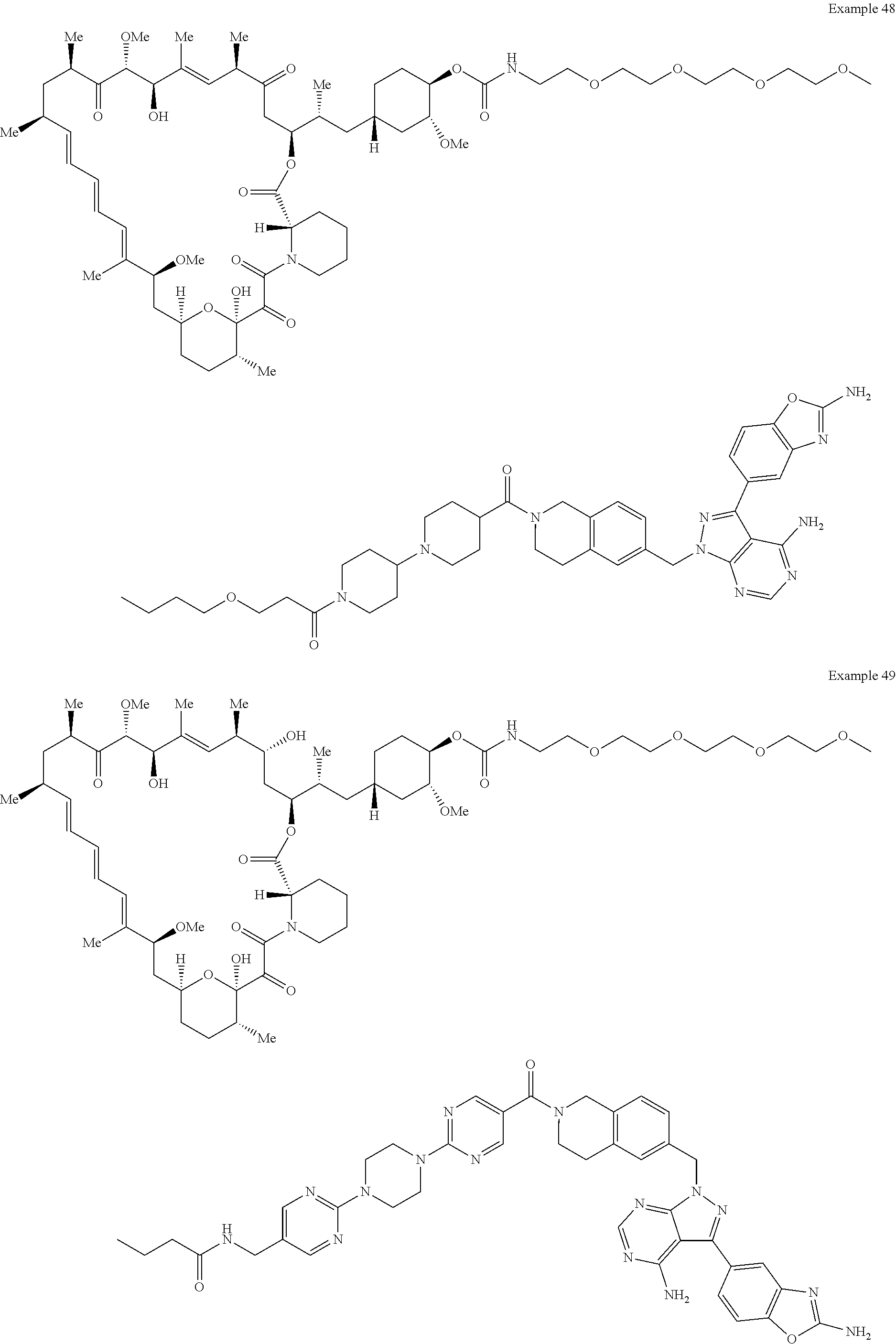

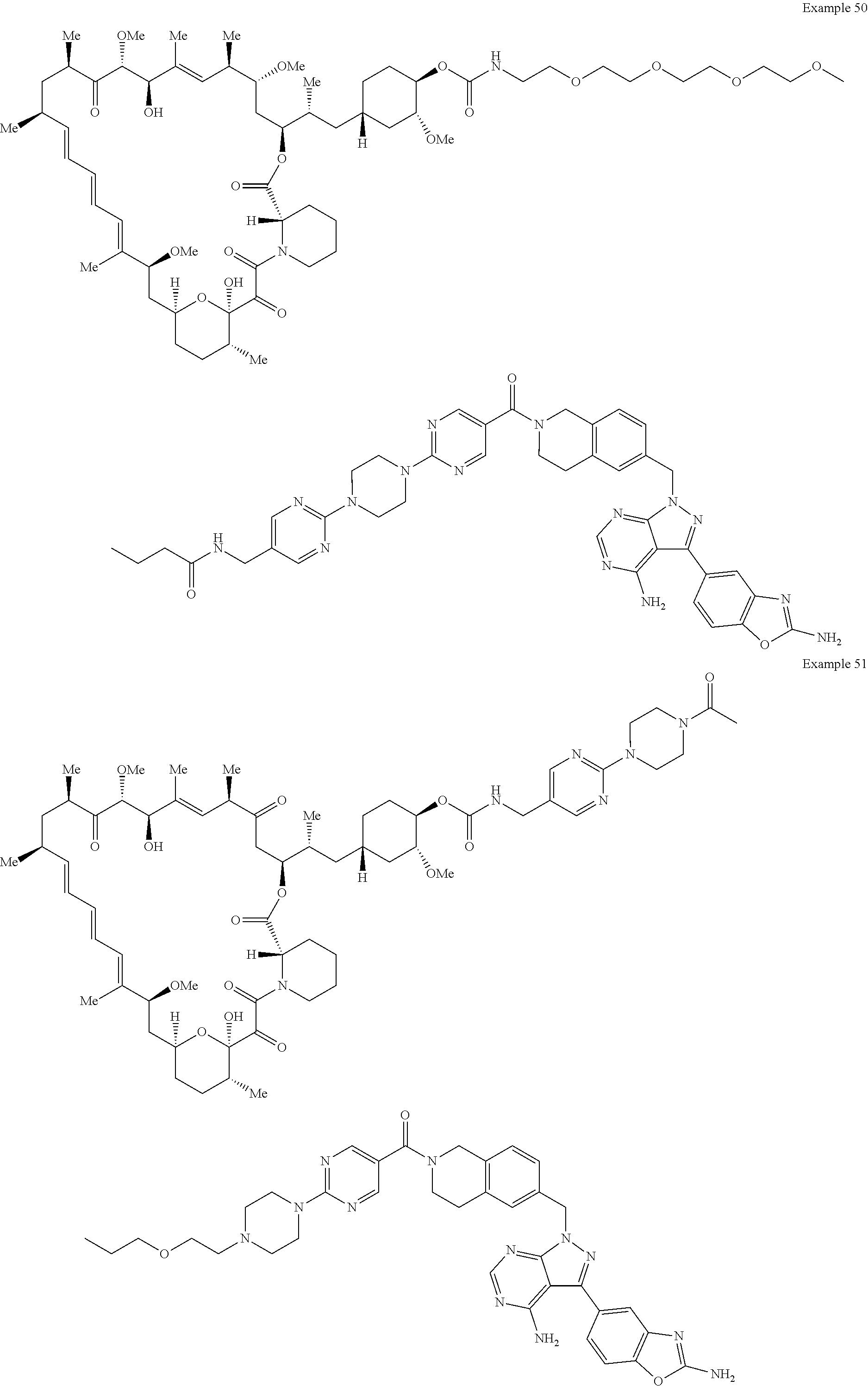

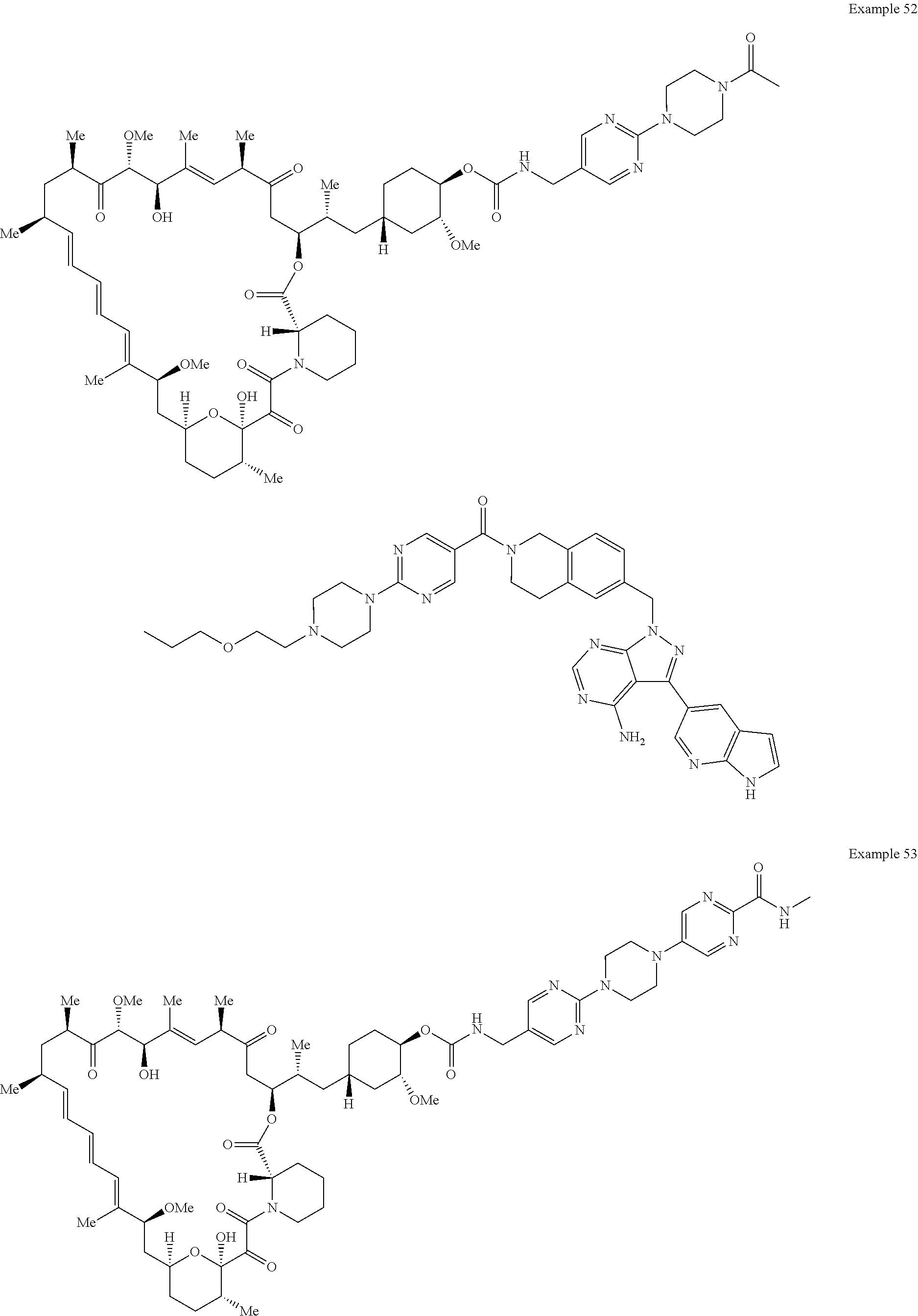

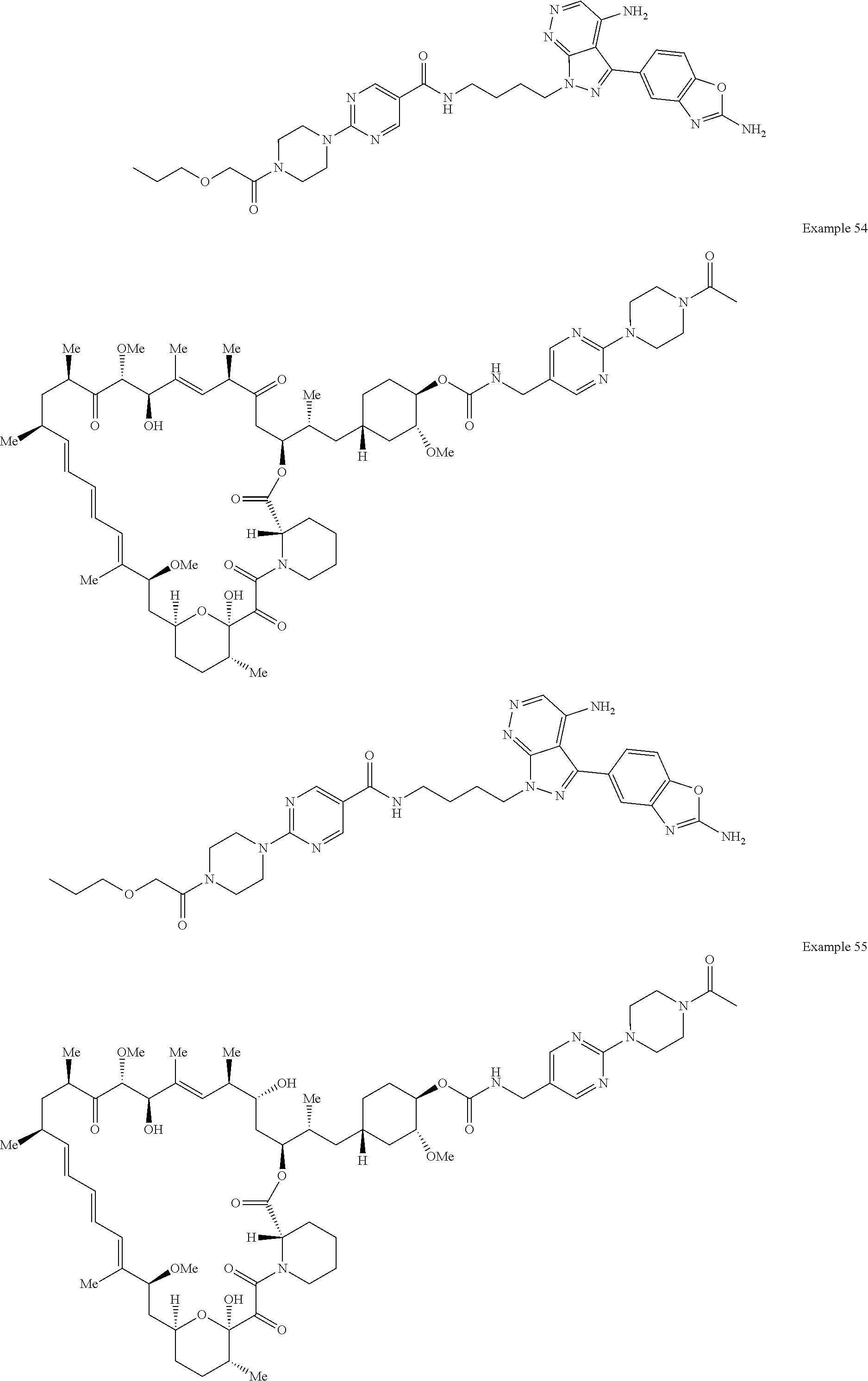

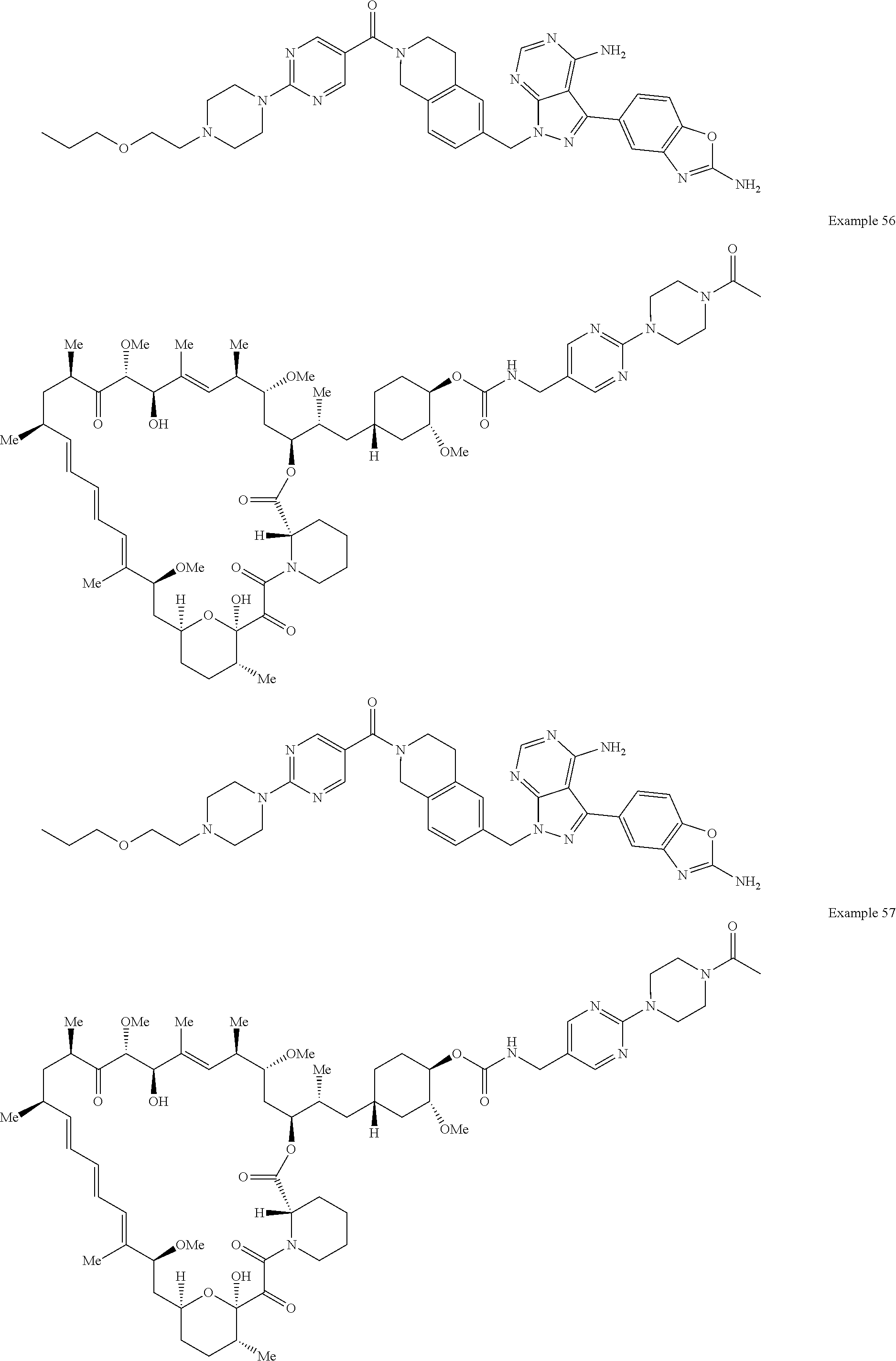

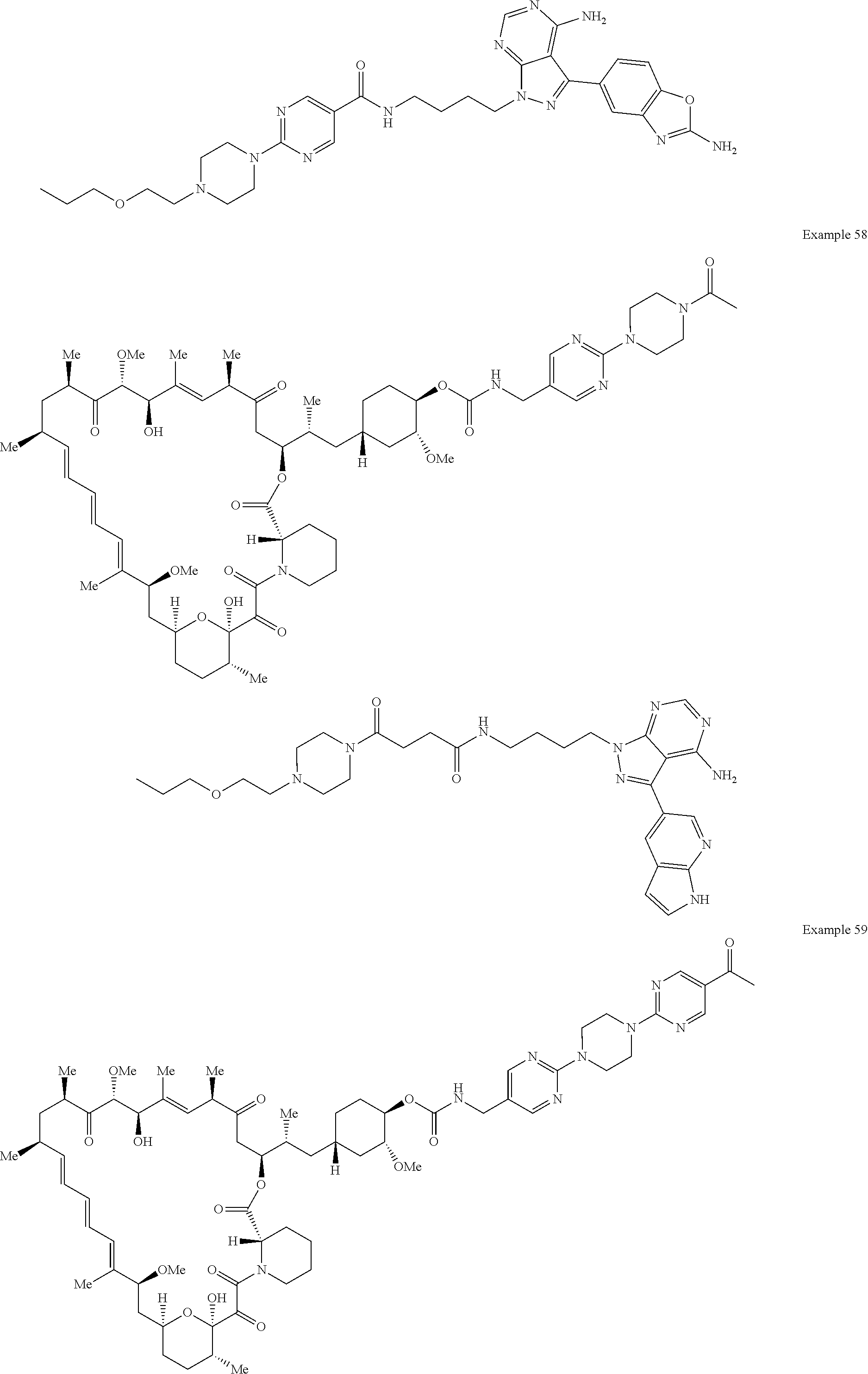

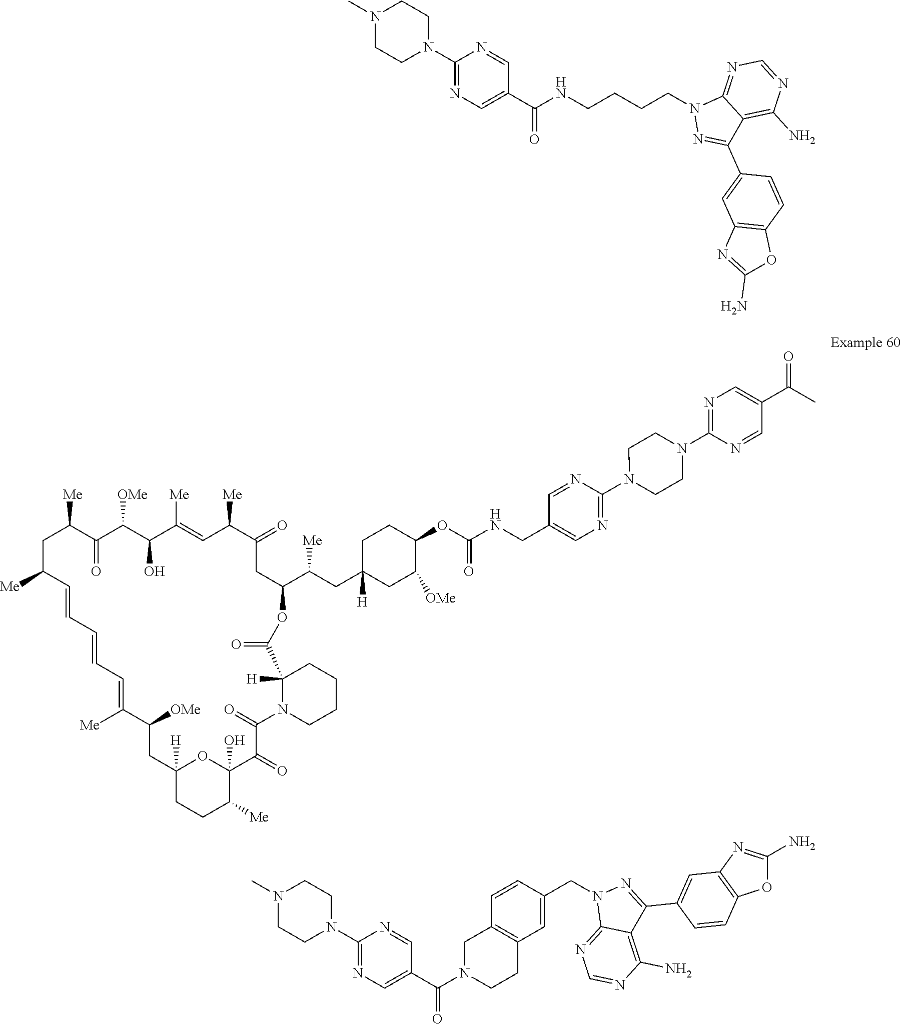

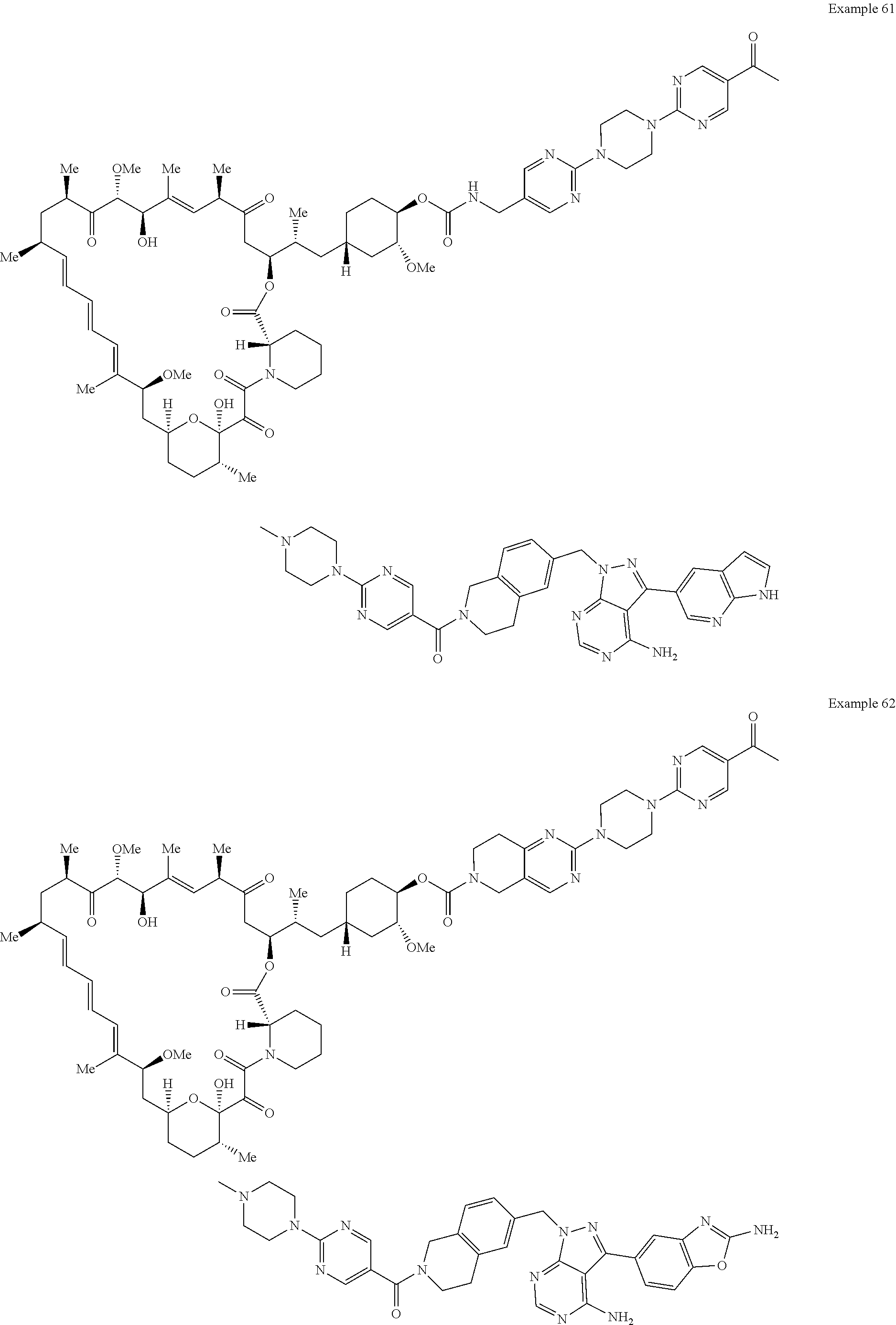

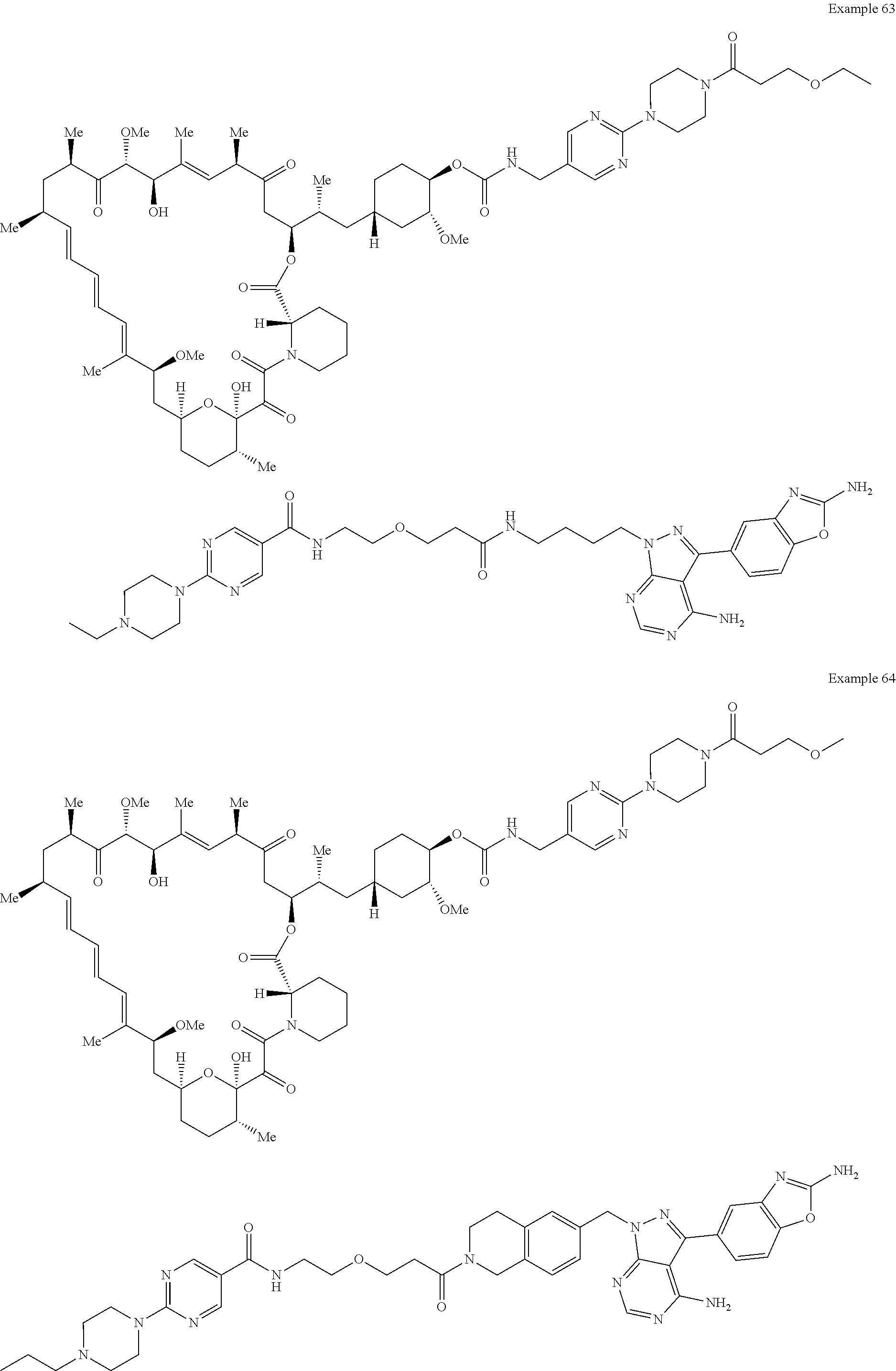

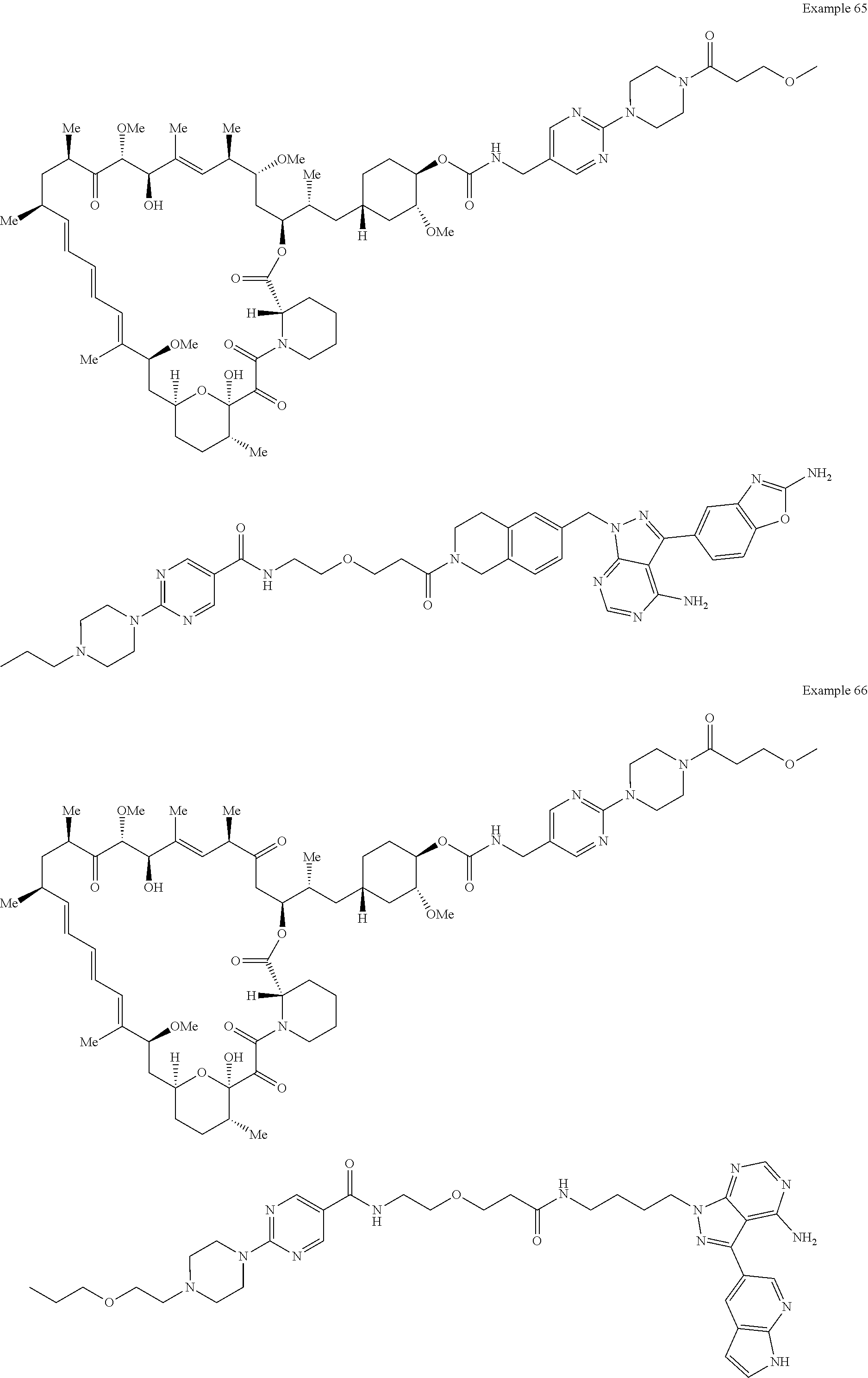

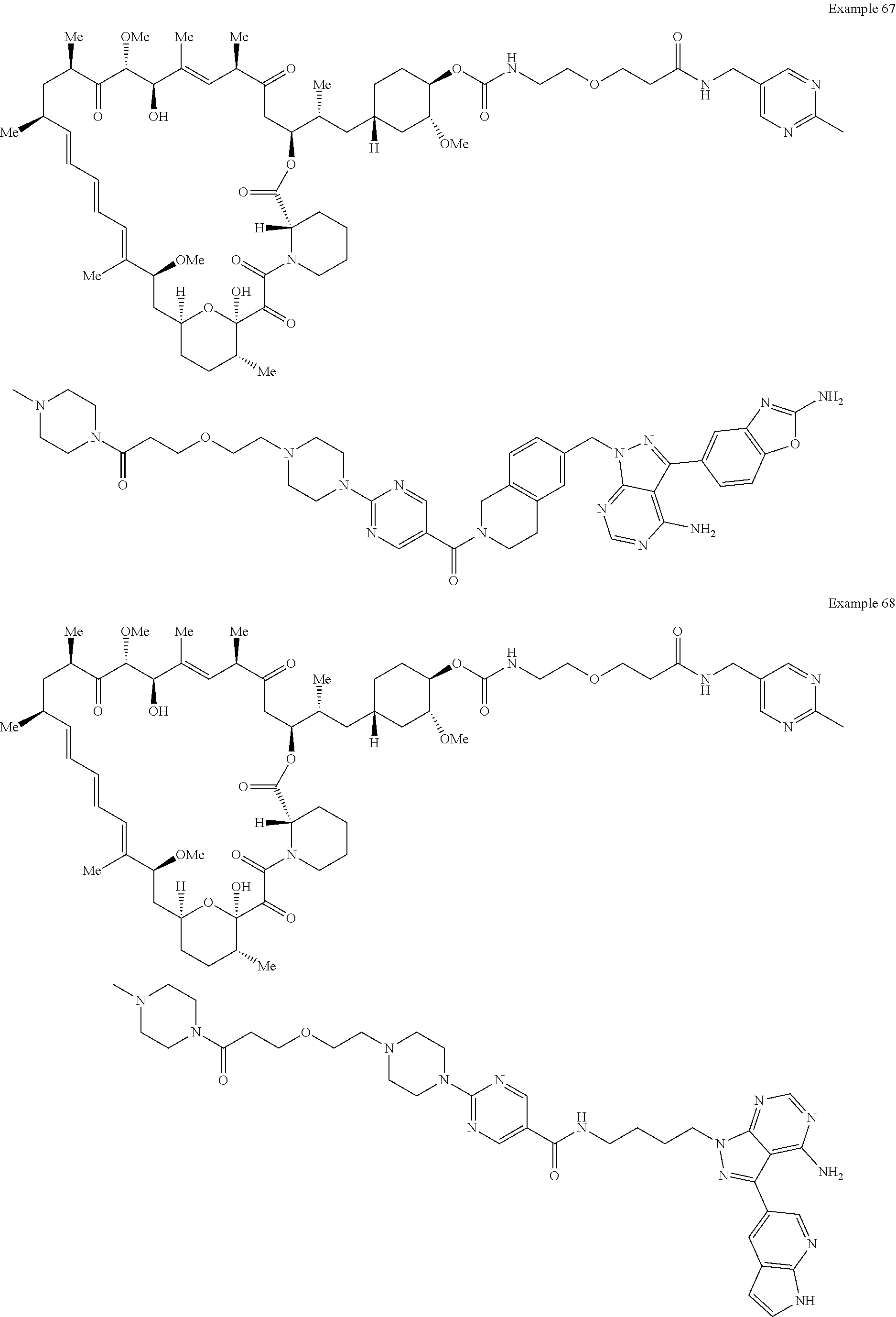

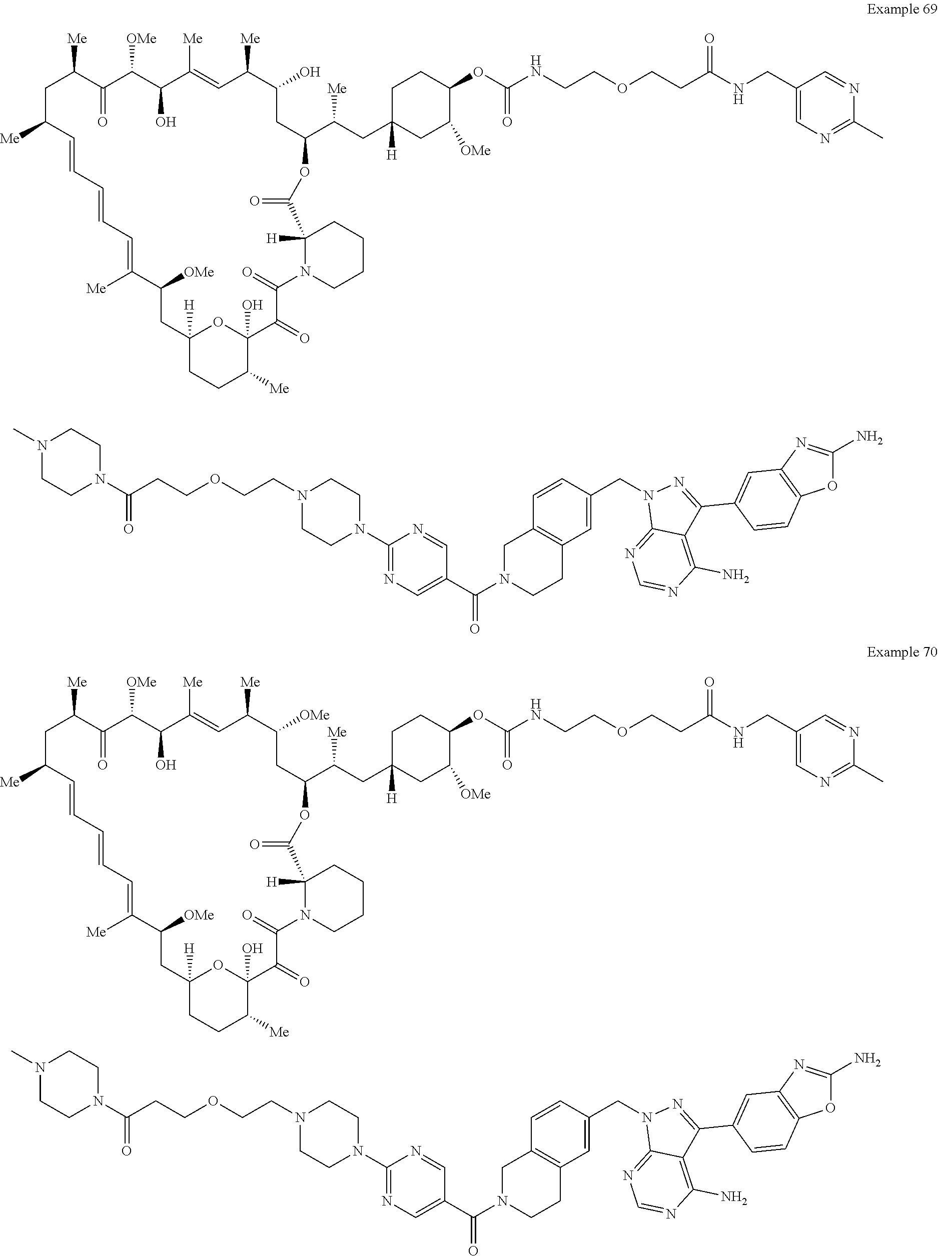

In certain embodiments, the present disclosure provides for a compound selected from below or a pharmaceutically acceptable salt or tautomer thereof,

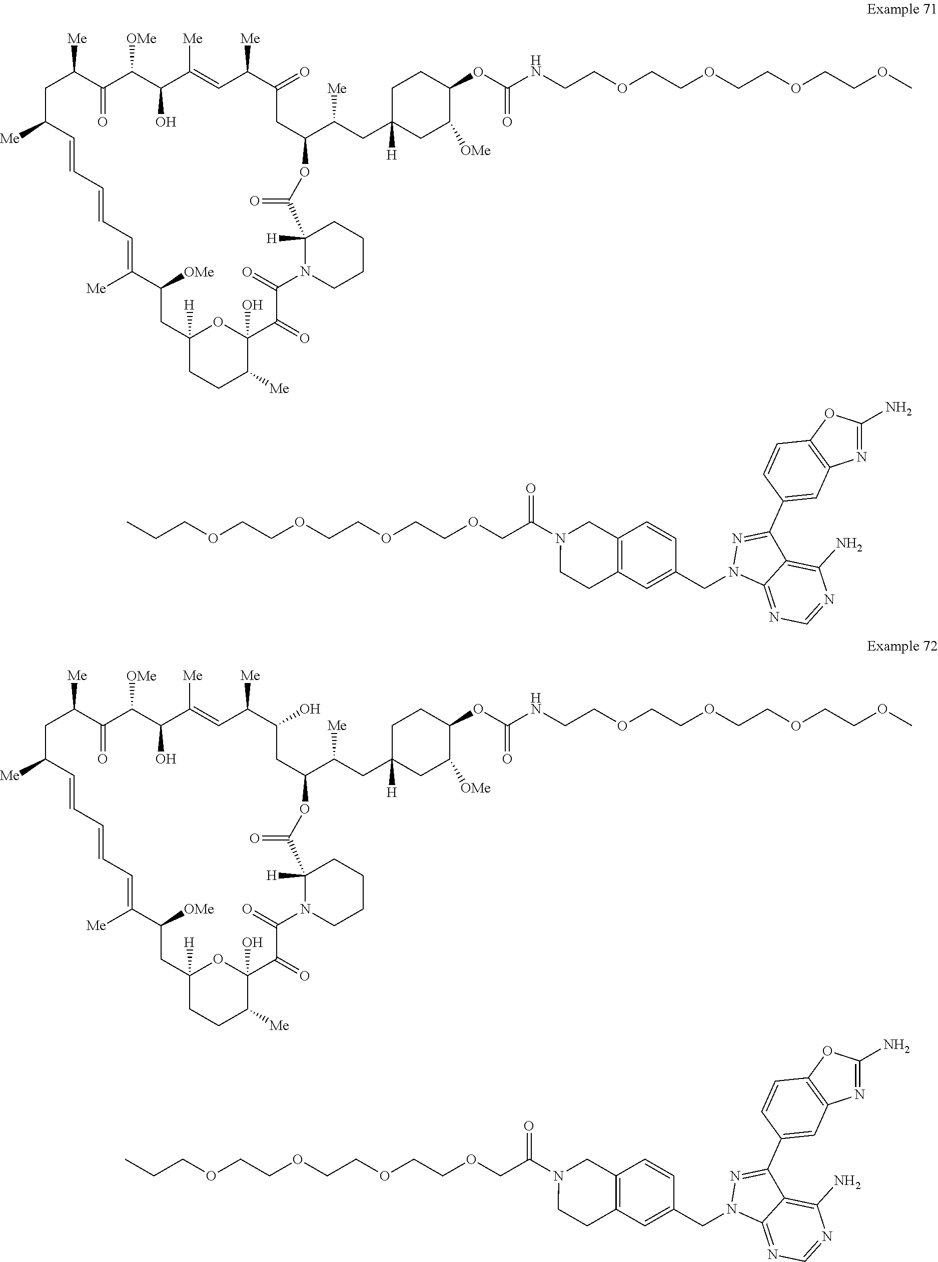

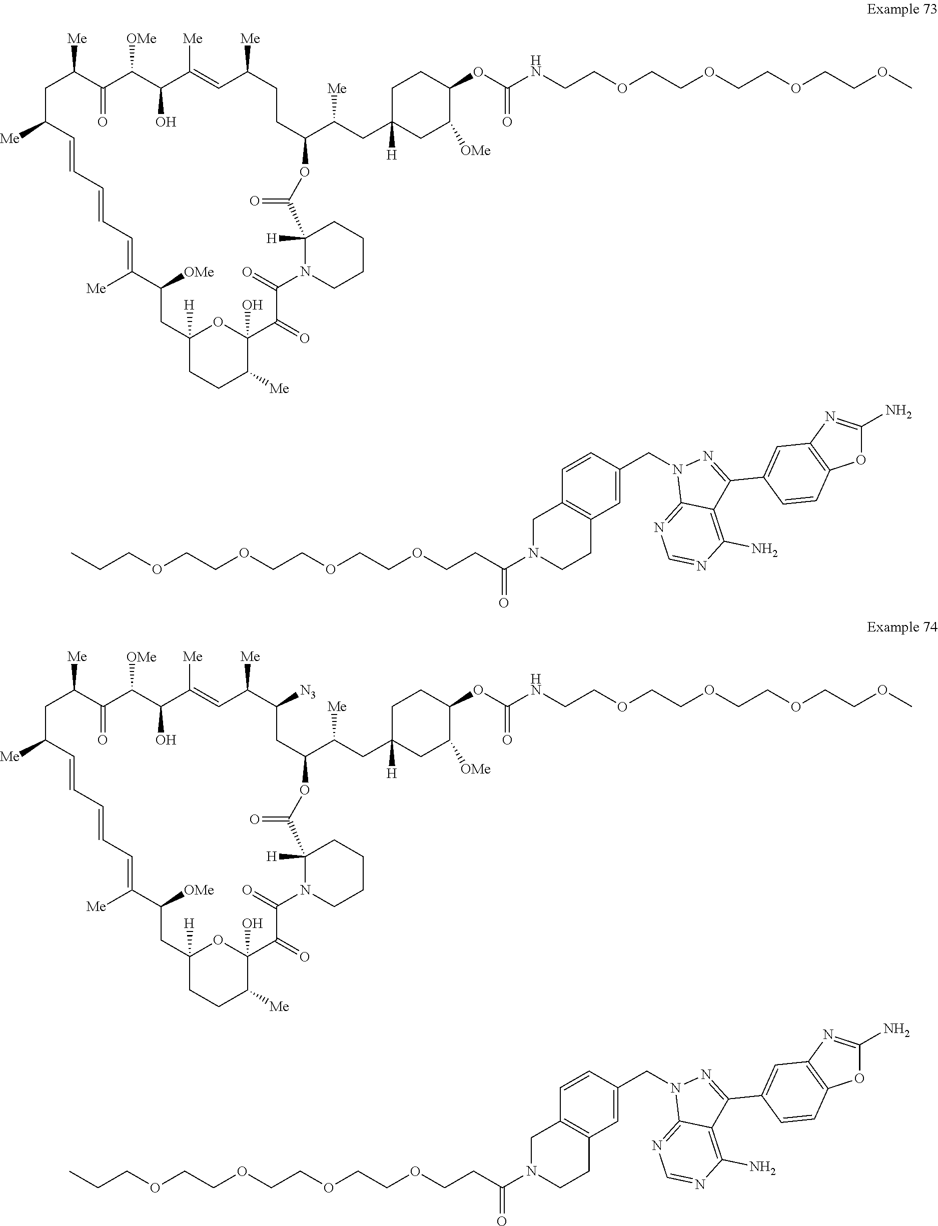

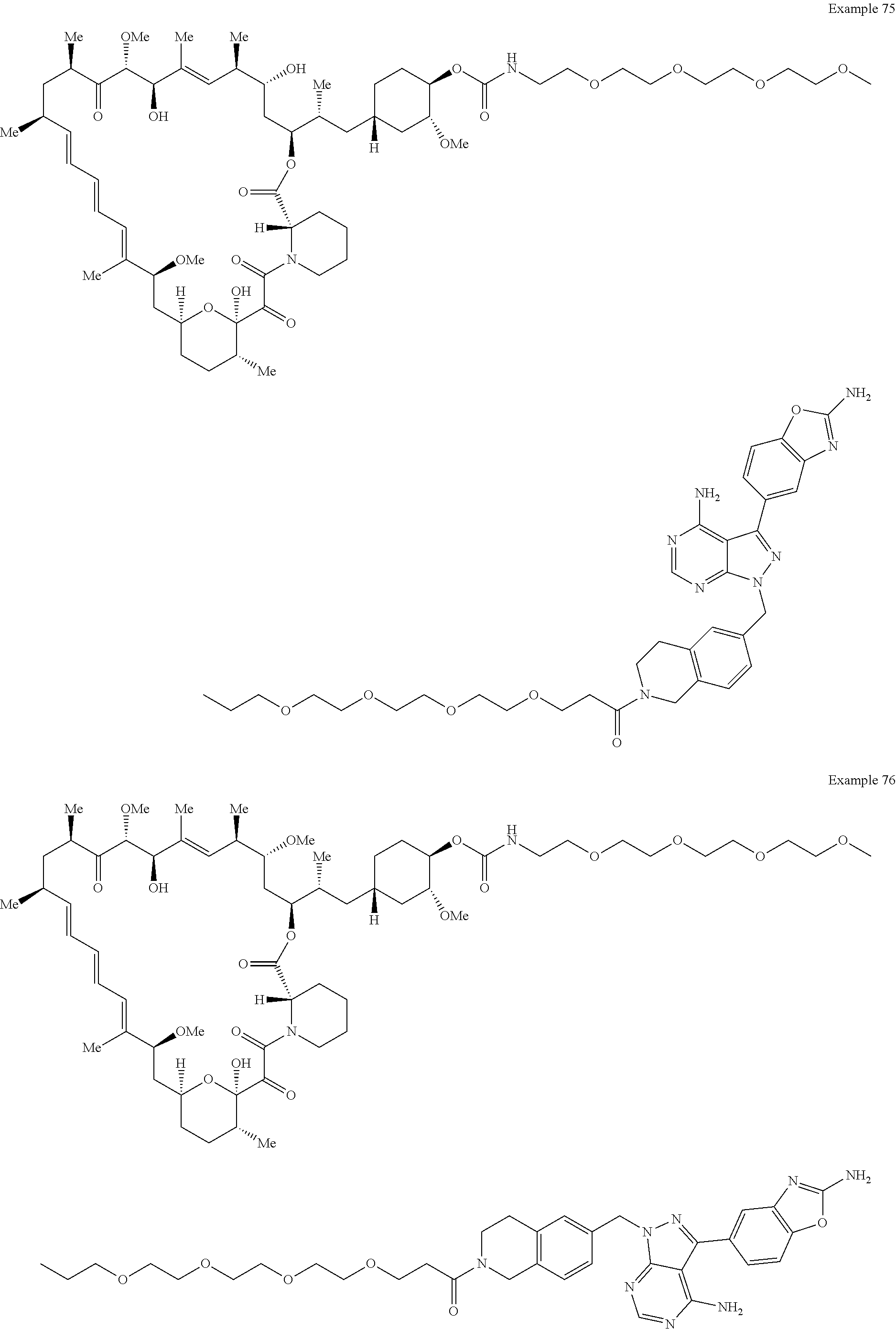

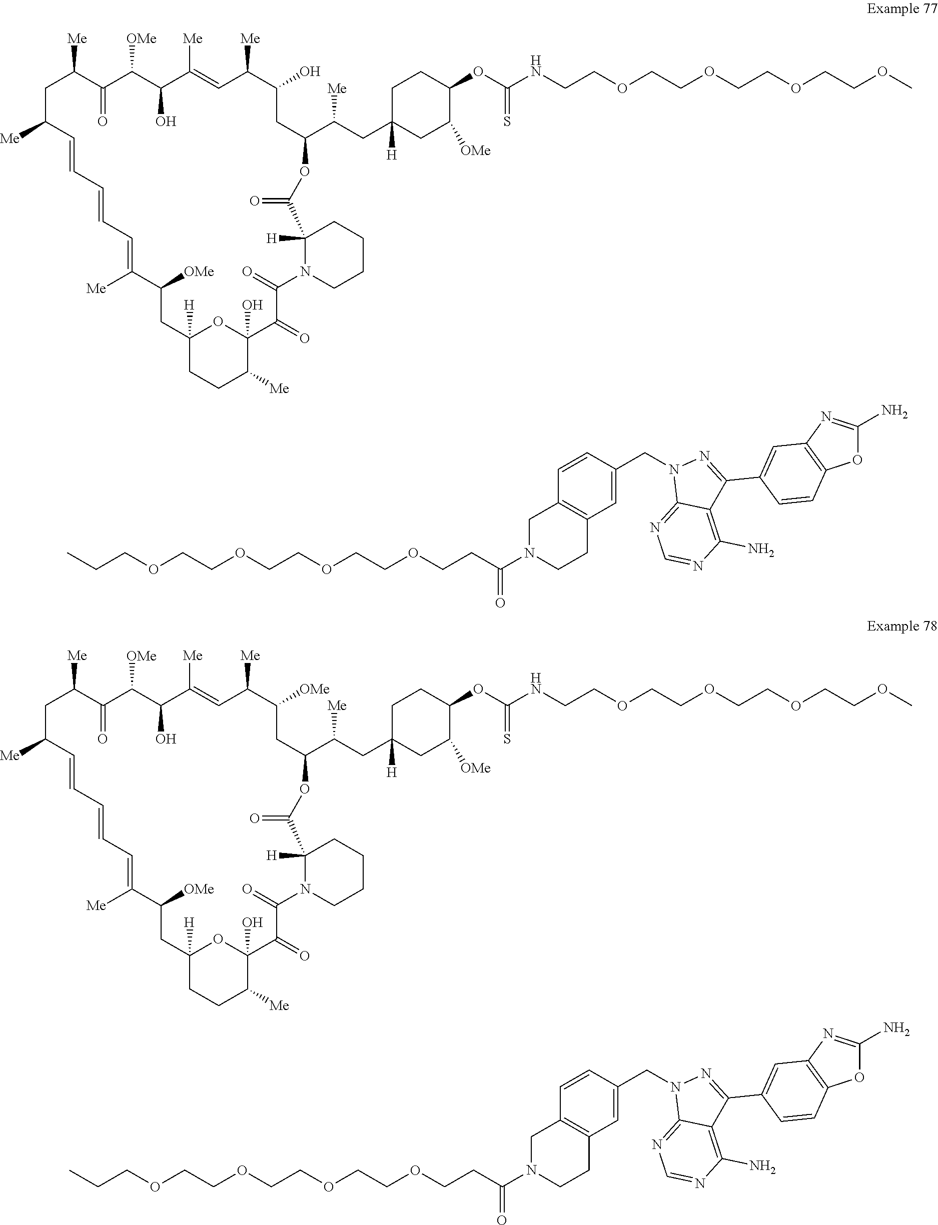

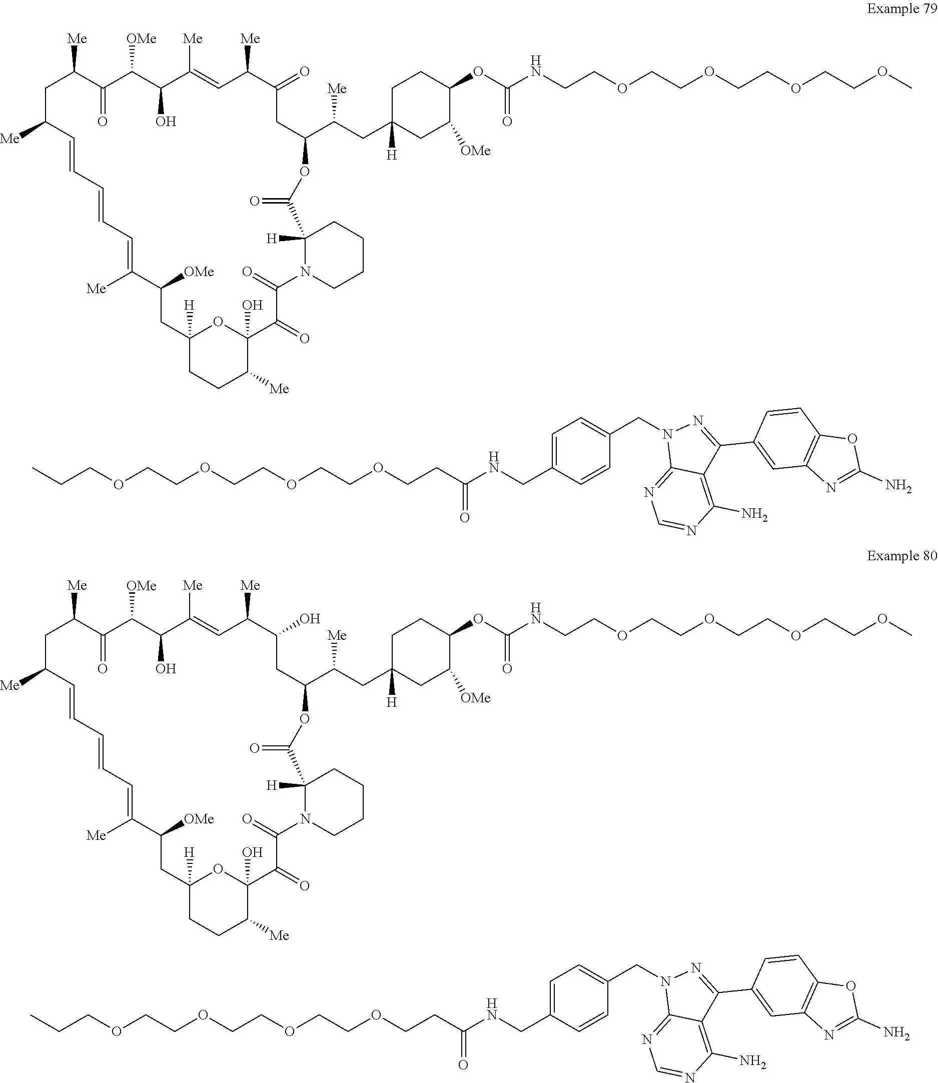

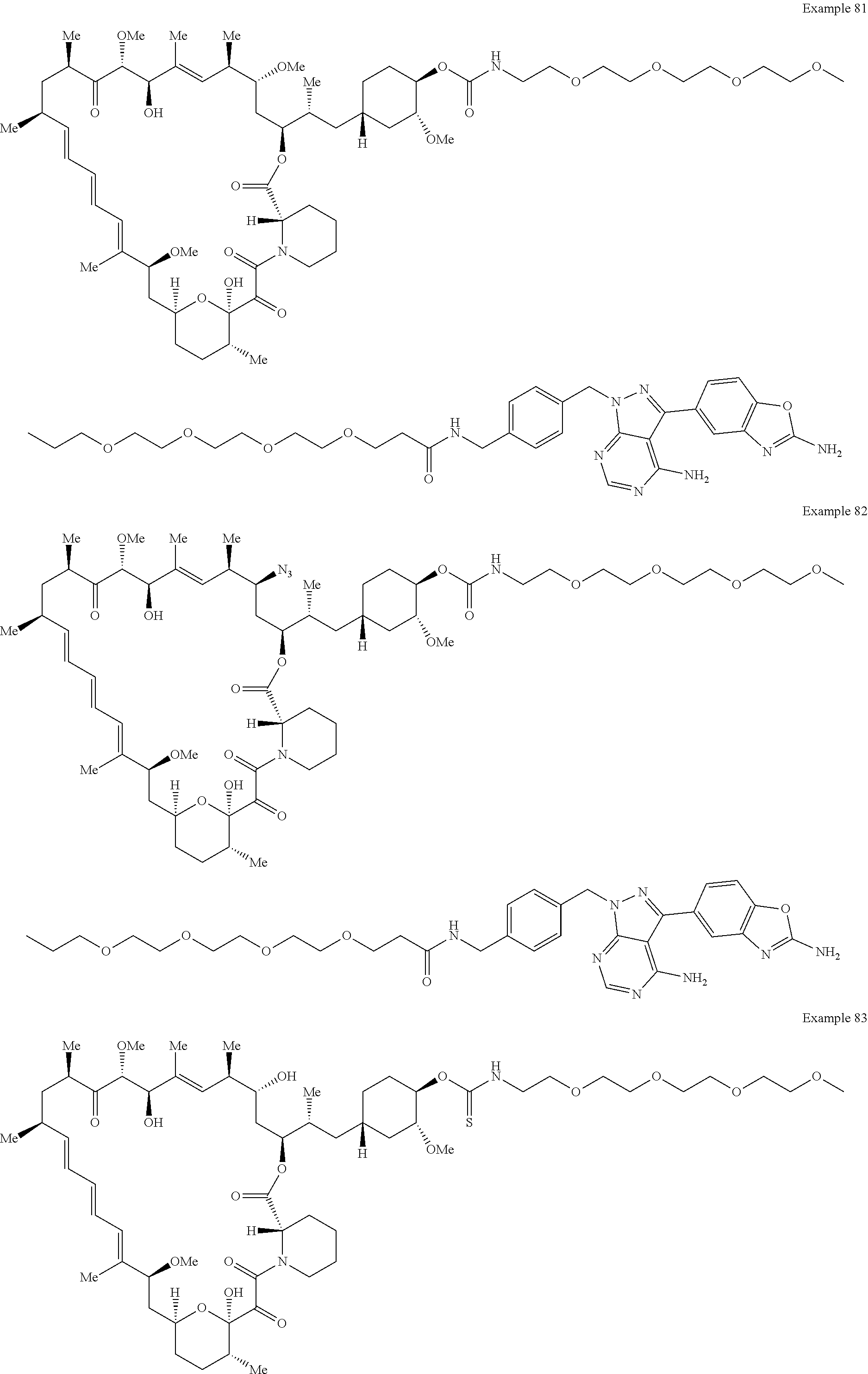

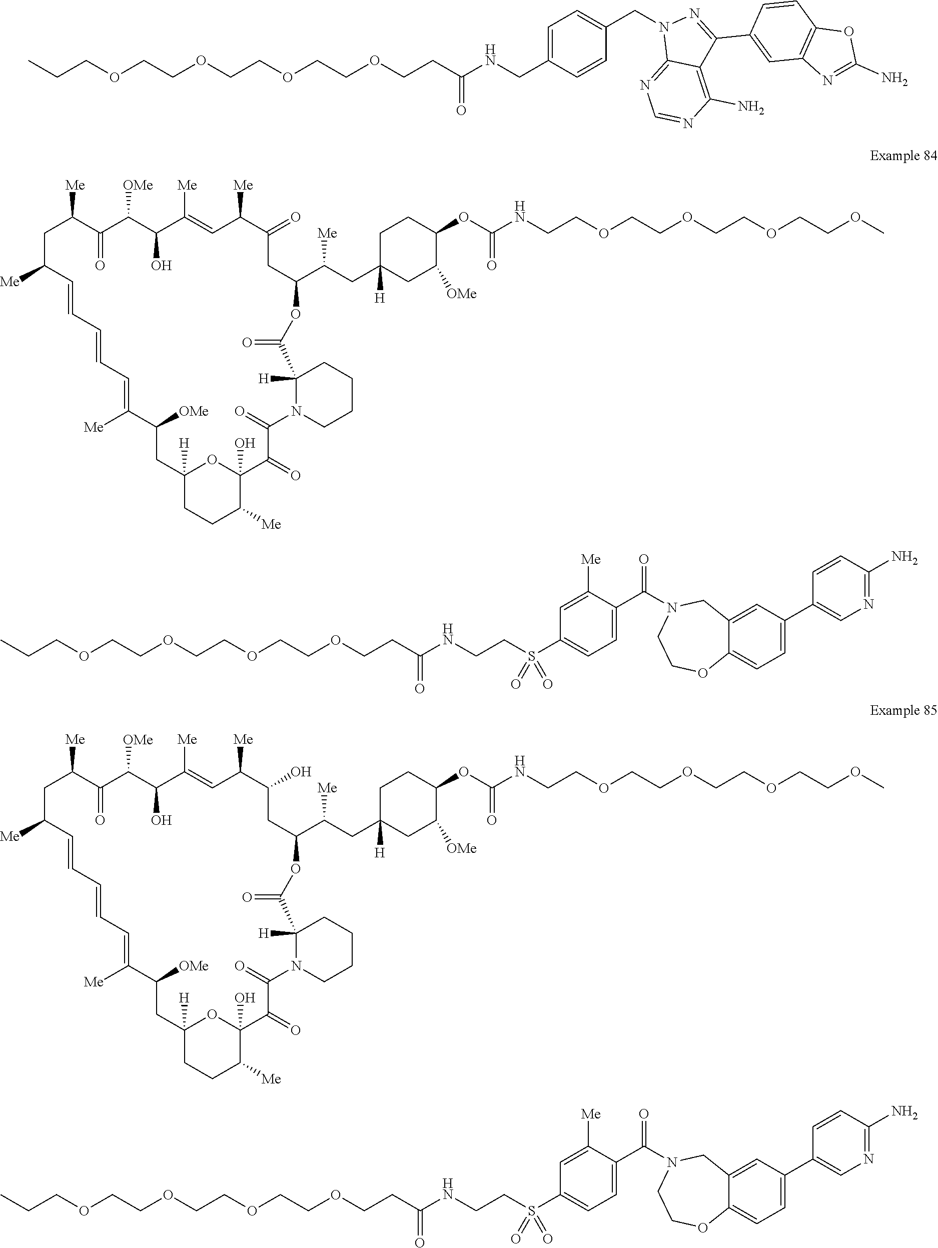

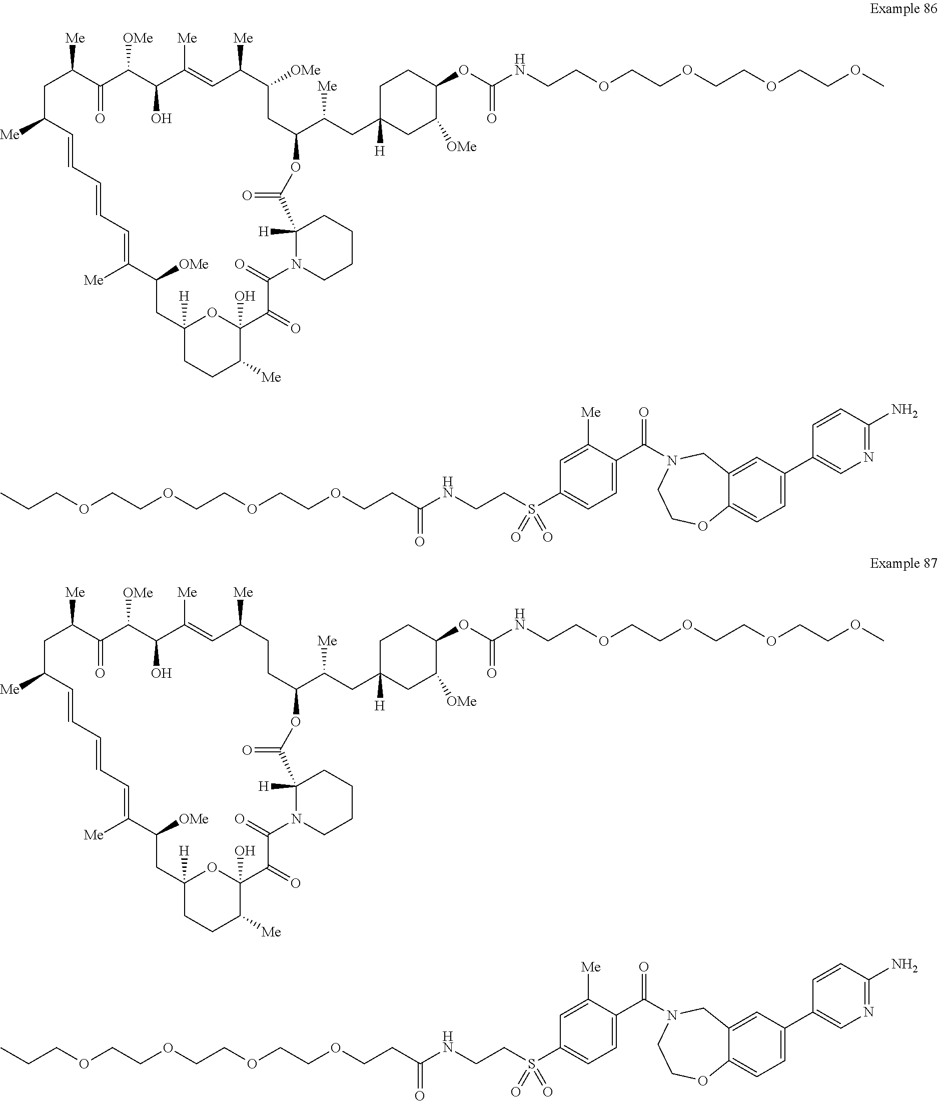

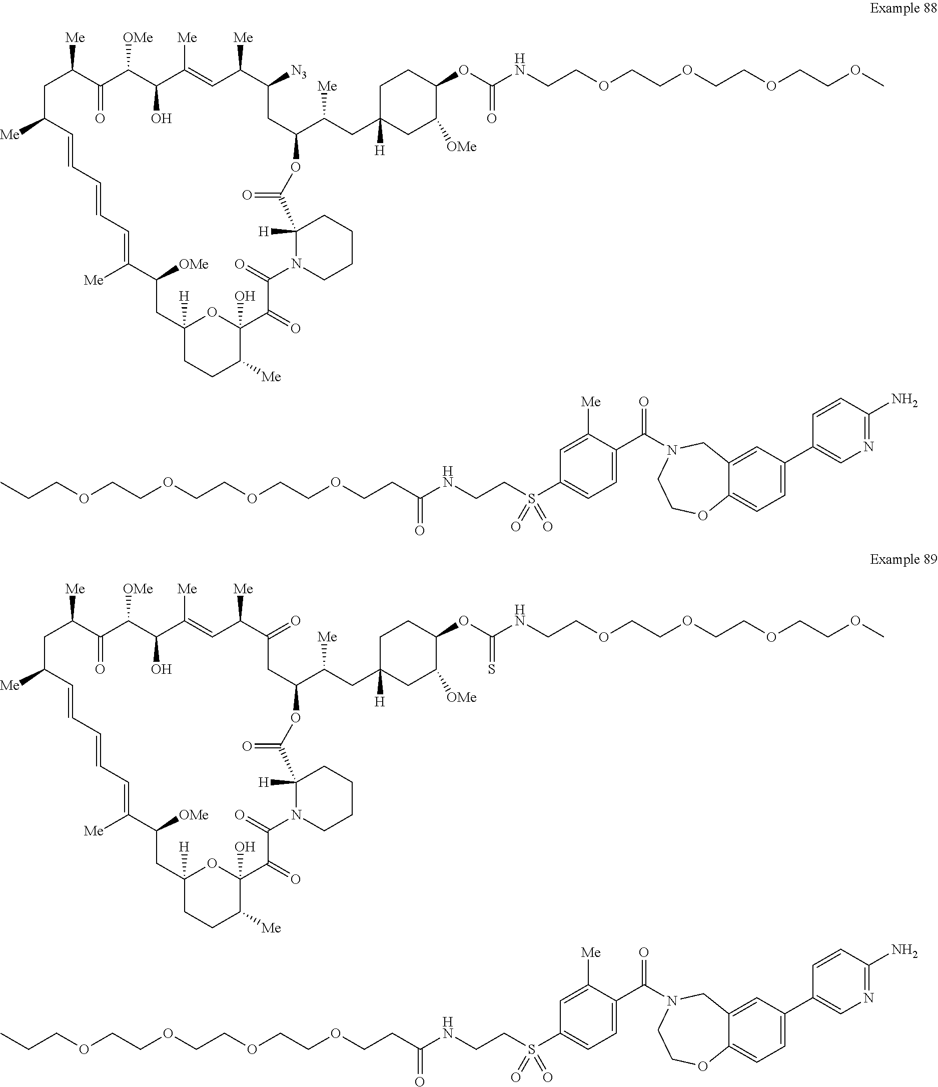

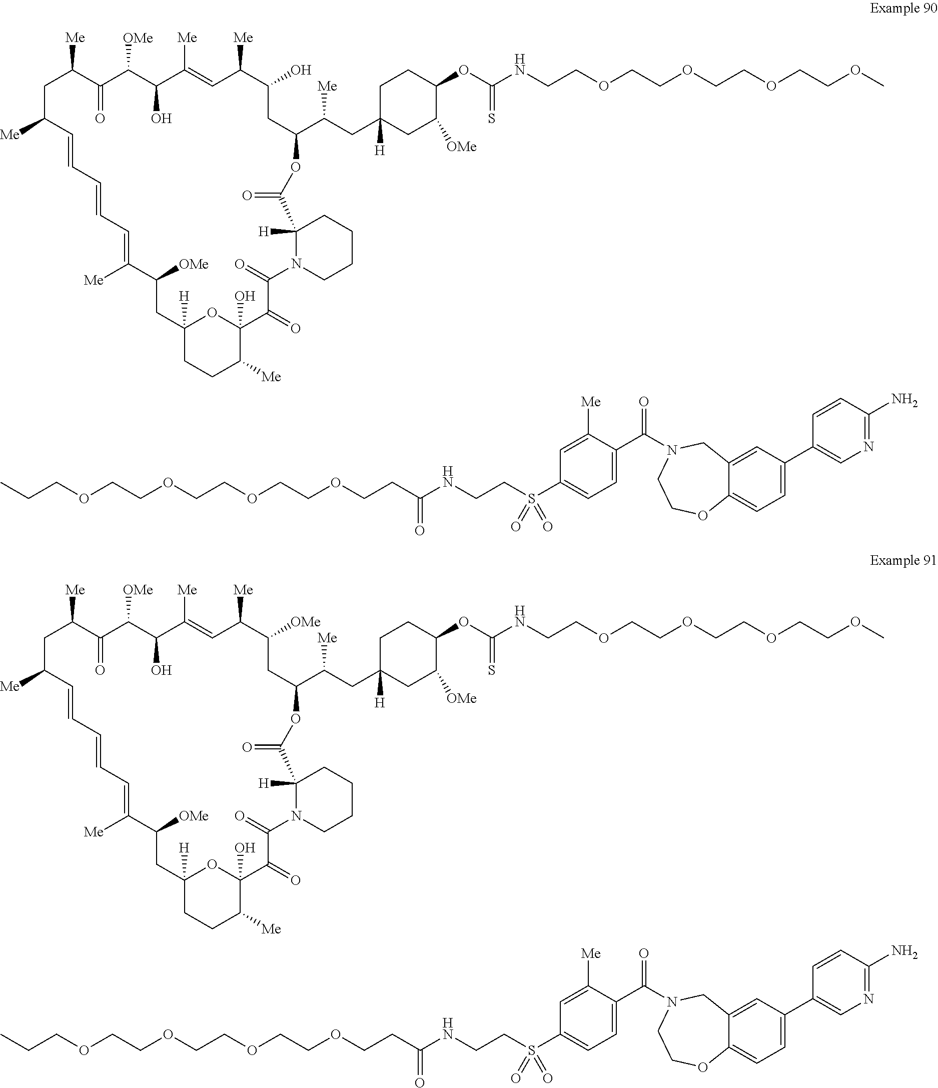

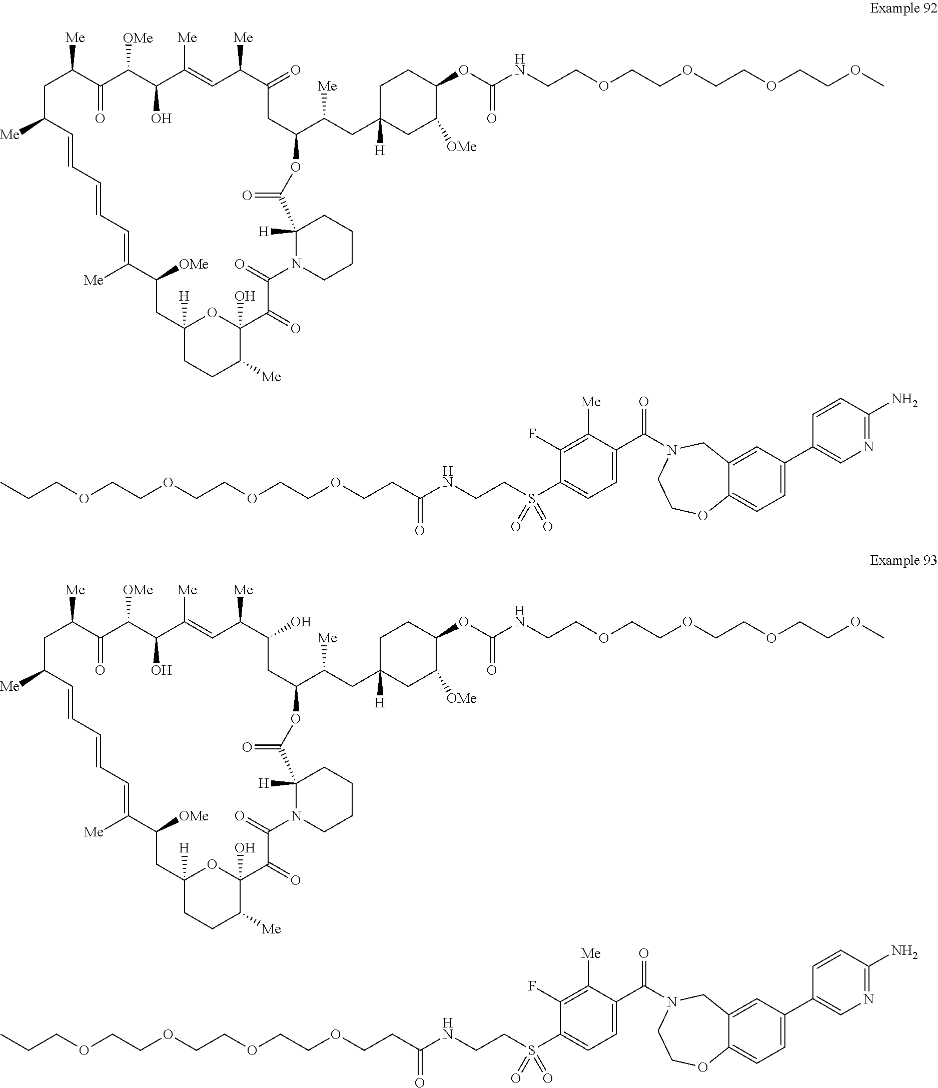

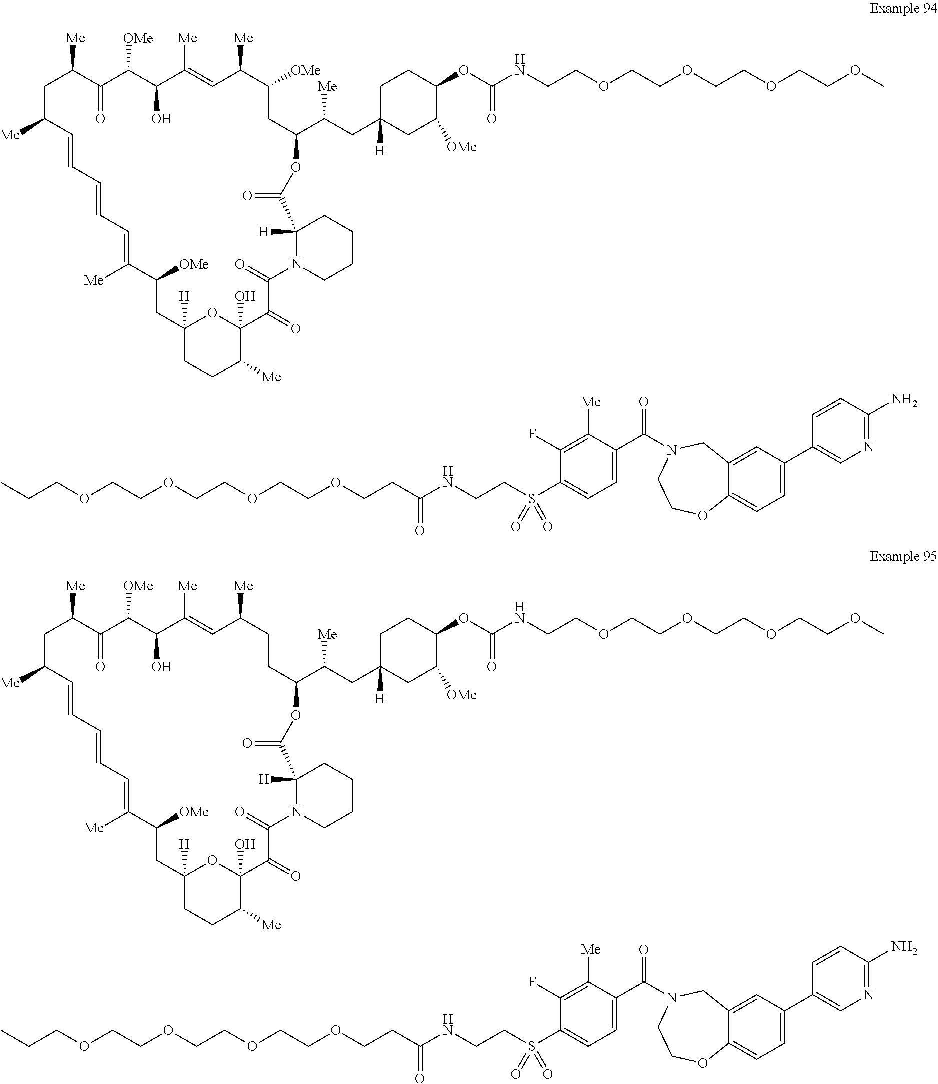

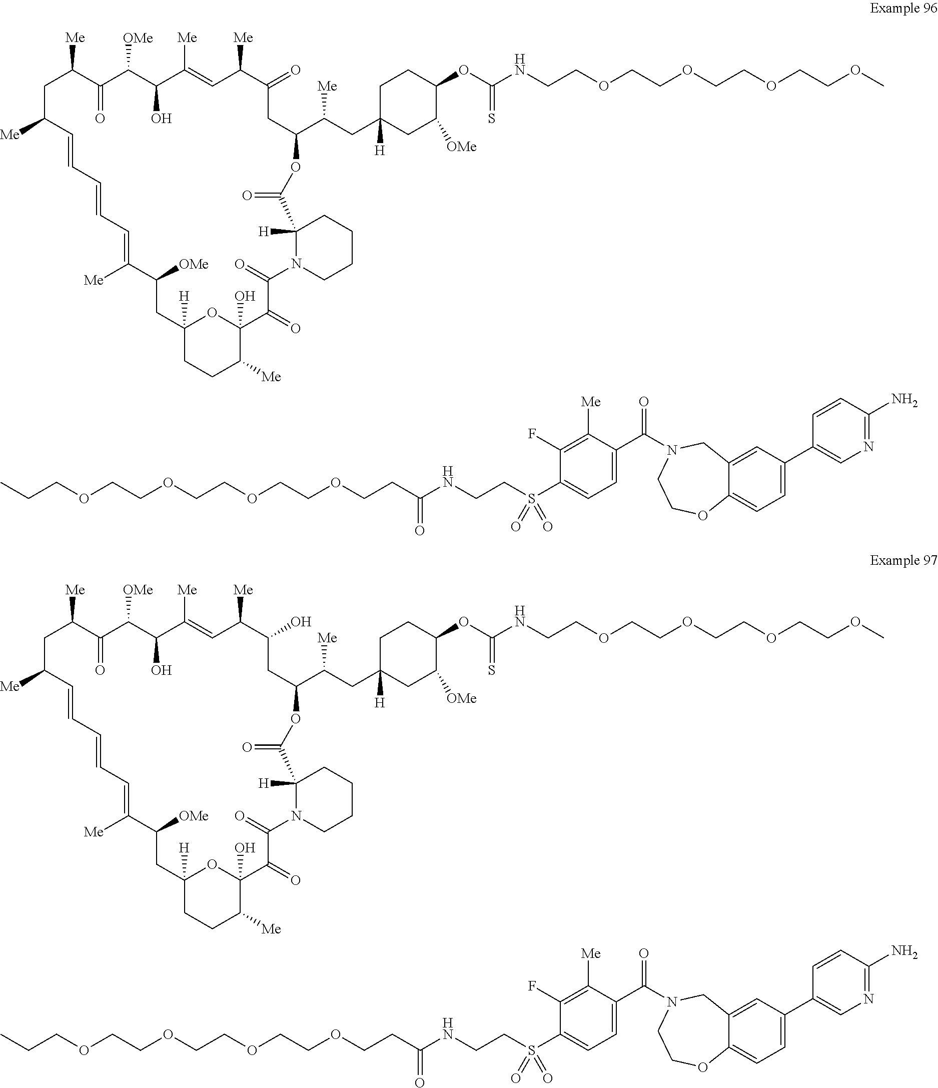

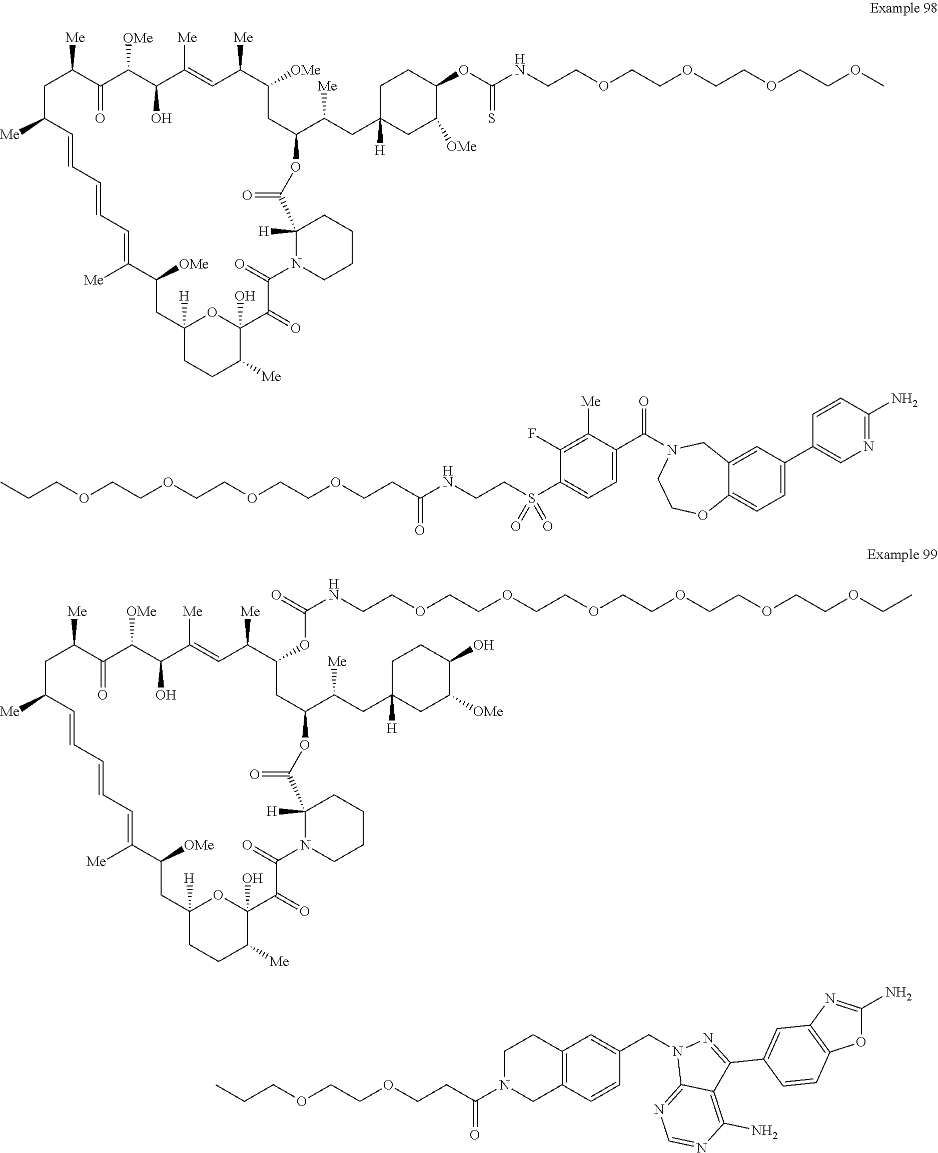

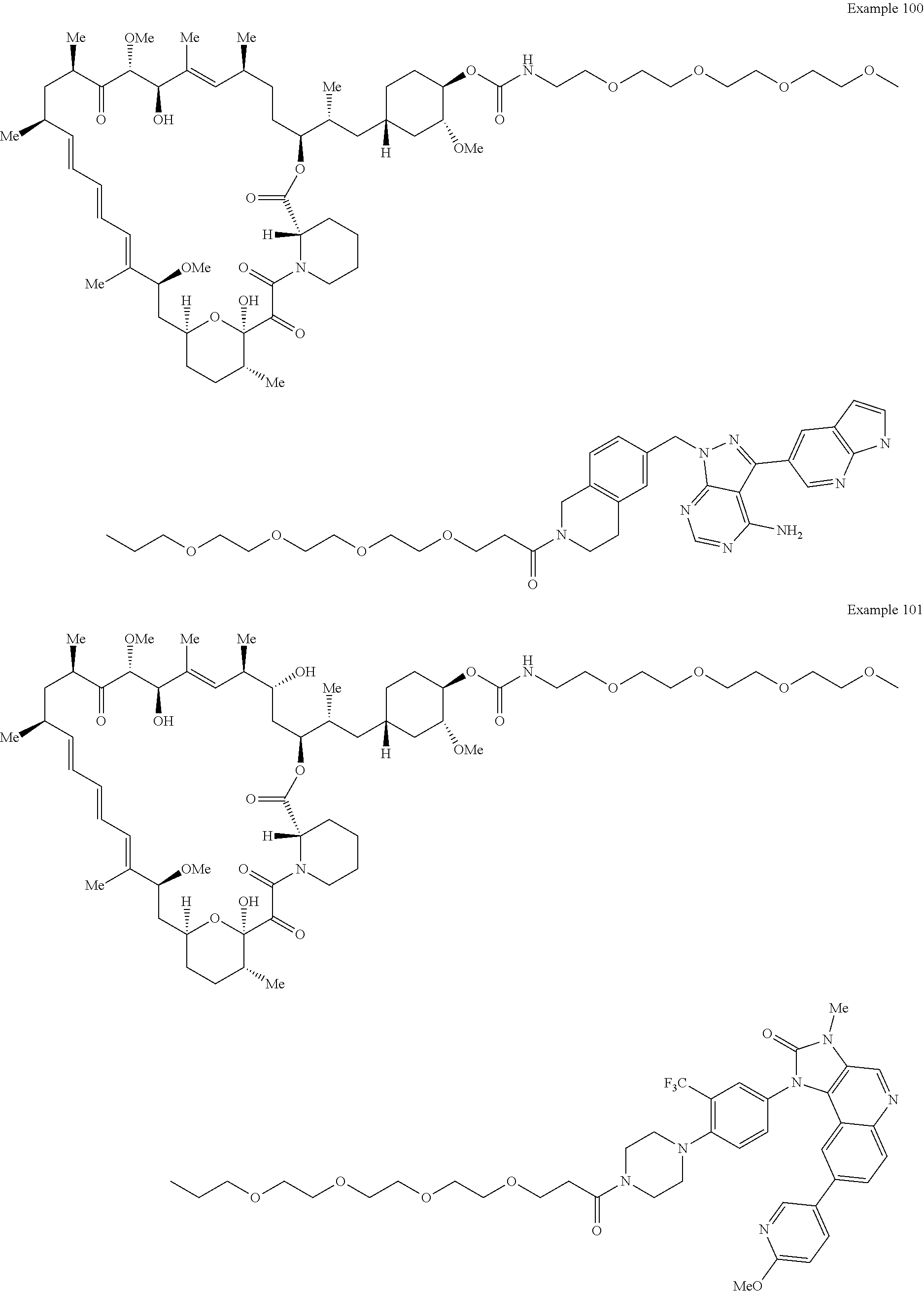

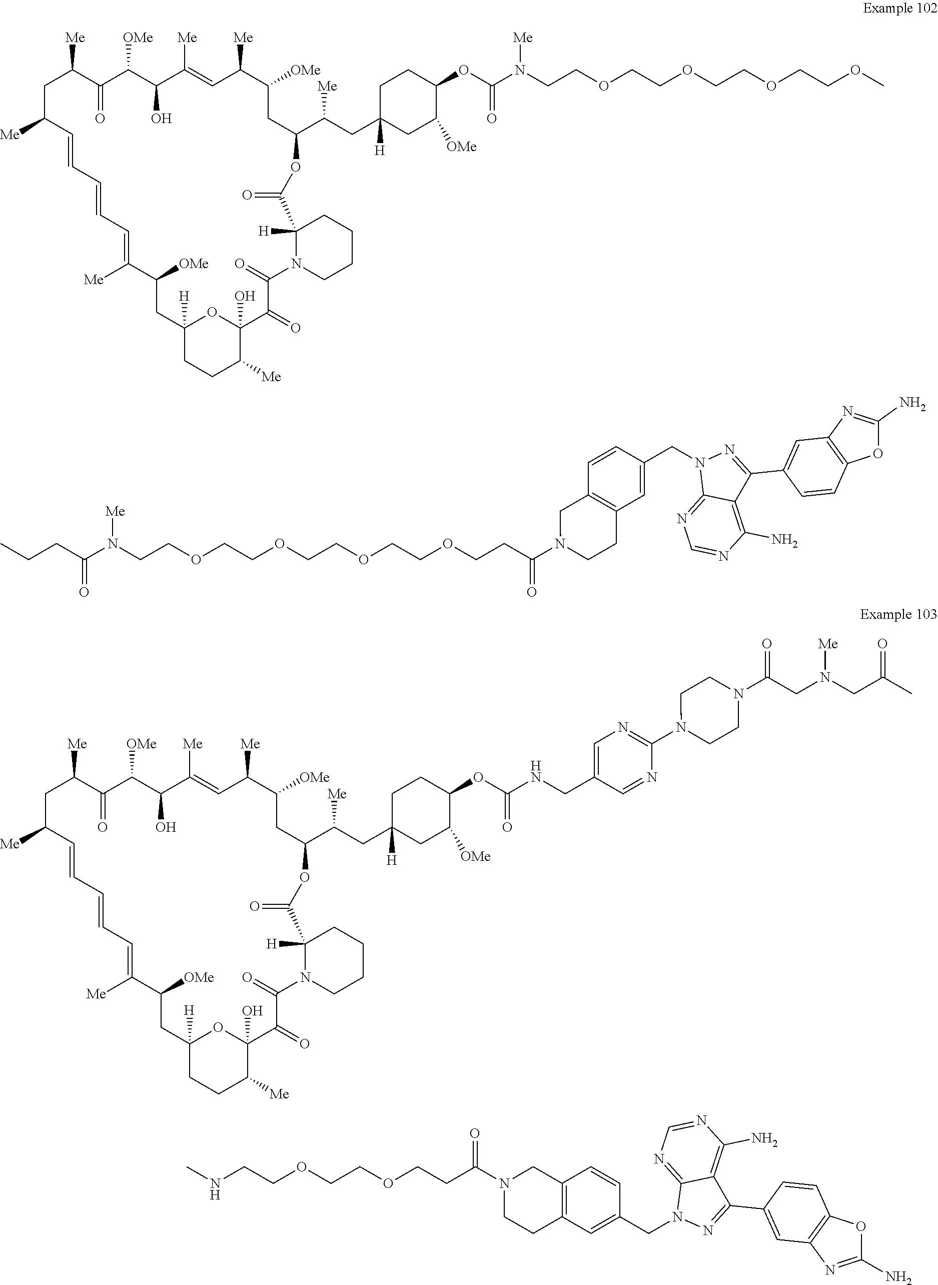

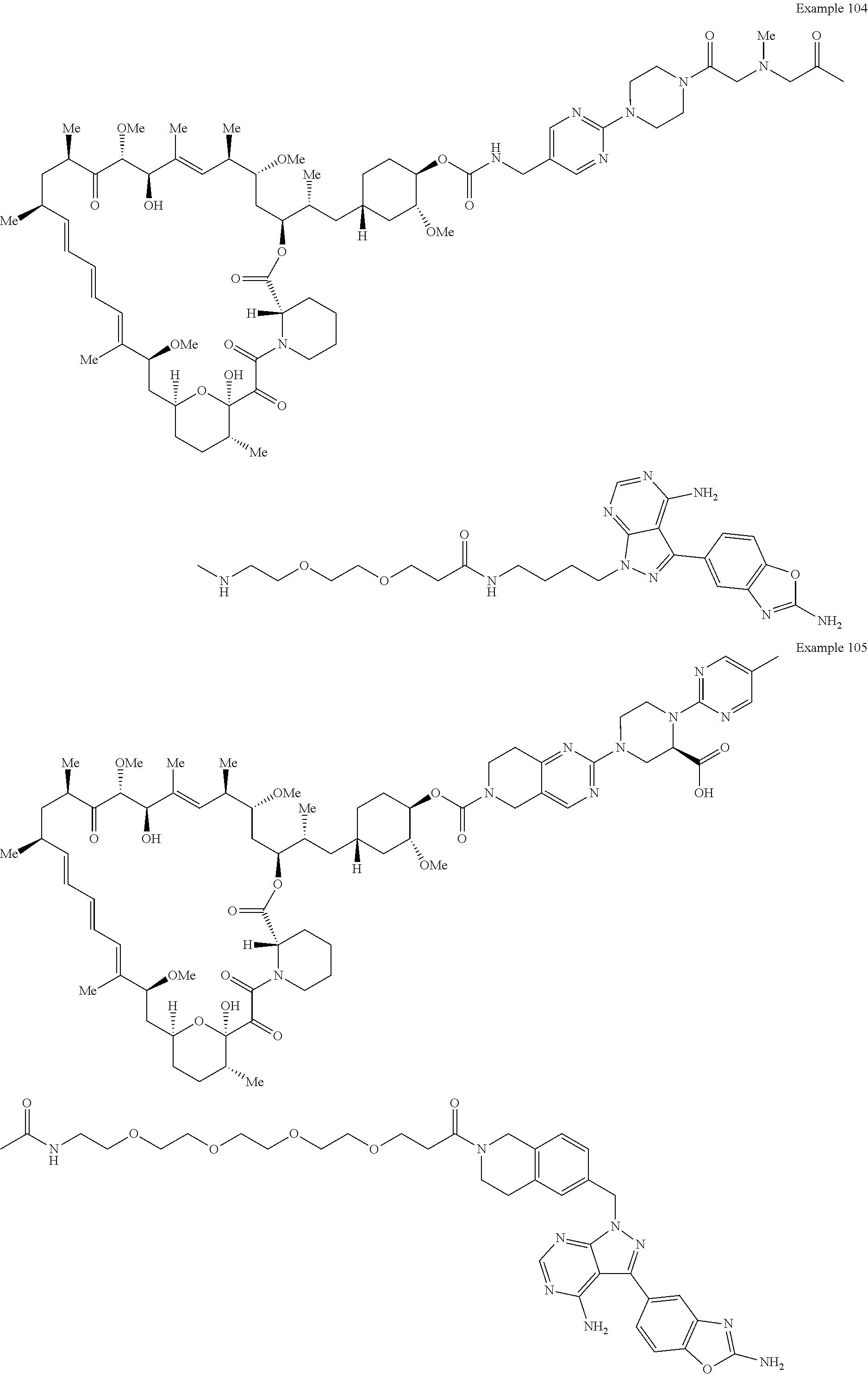

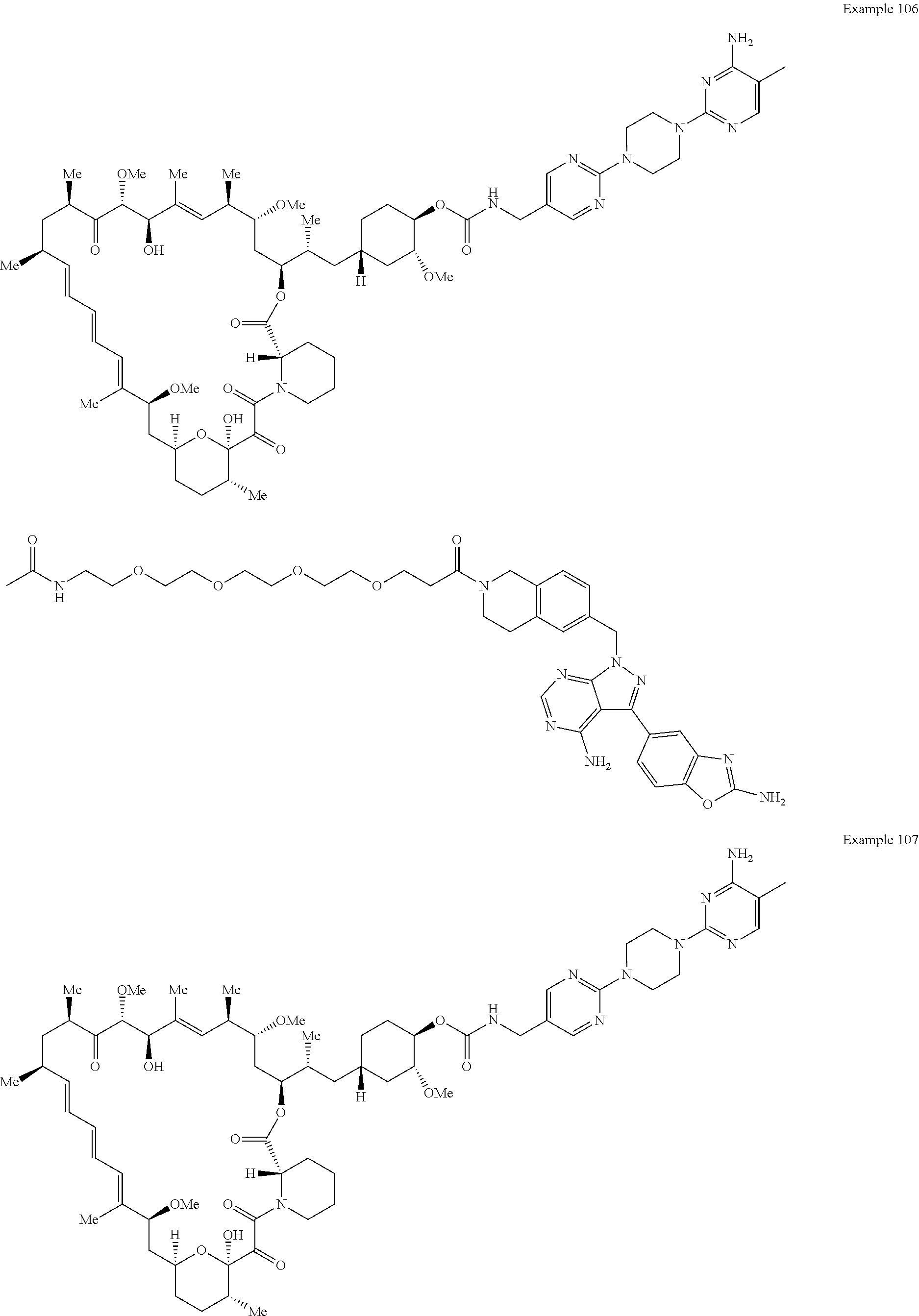

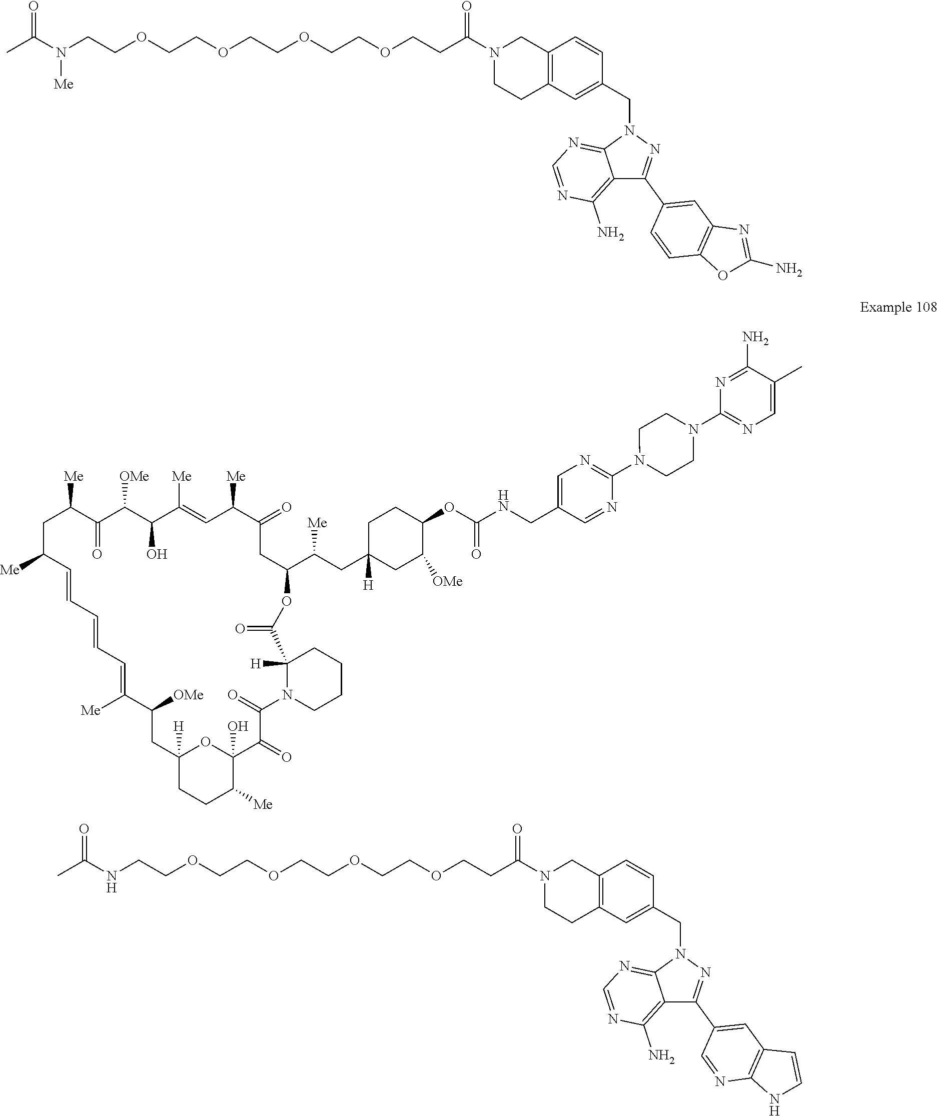

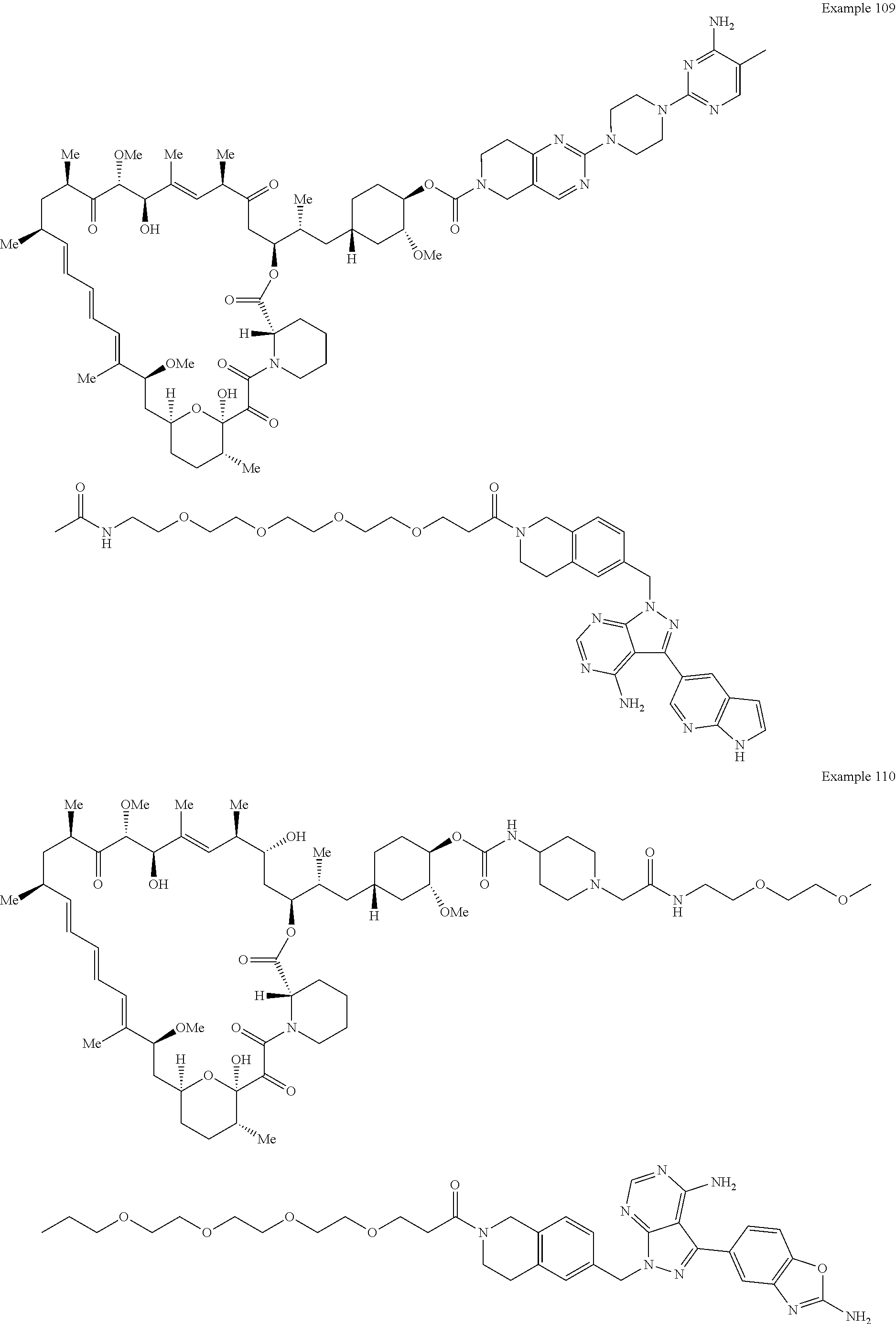

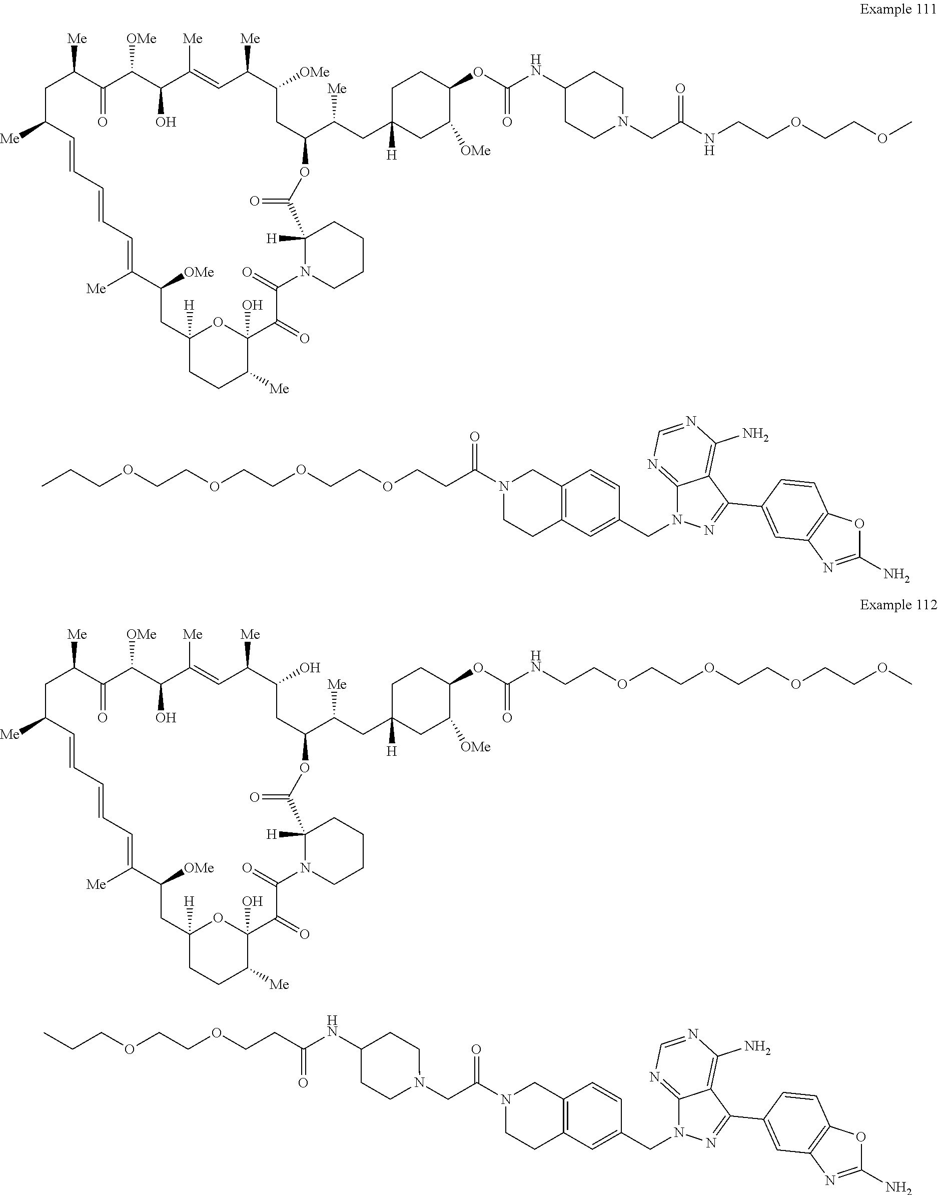

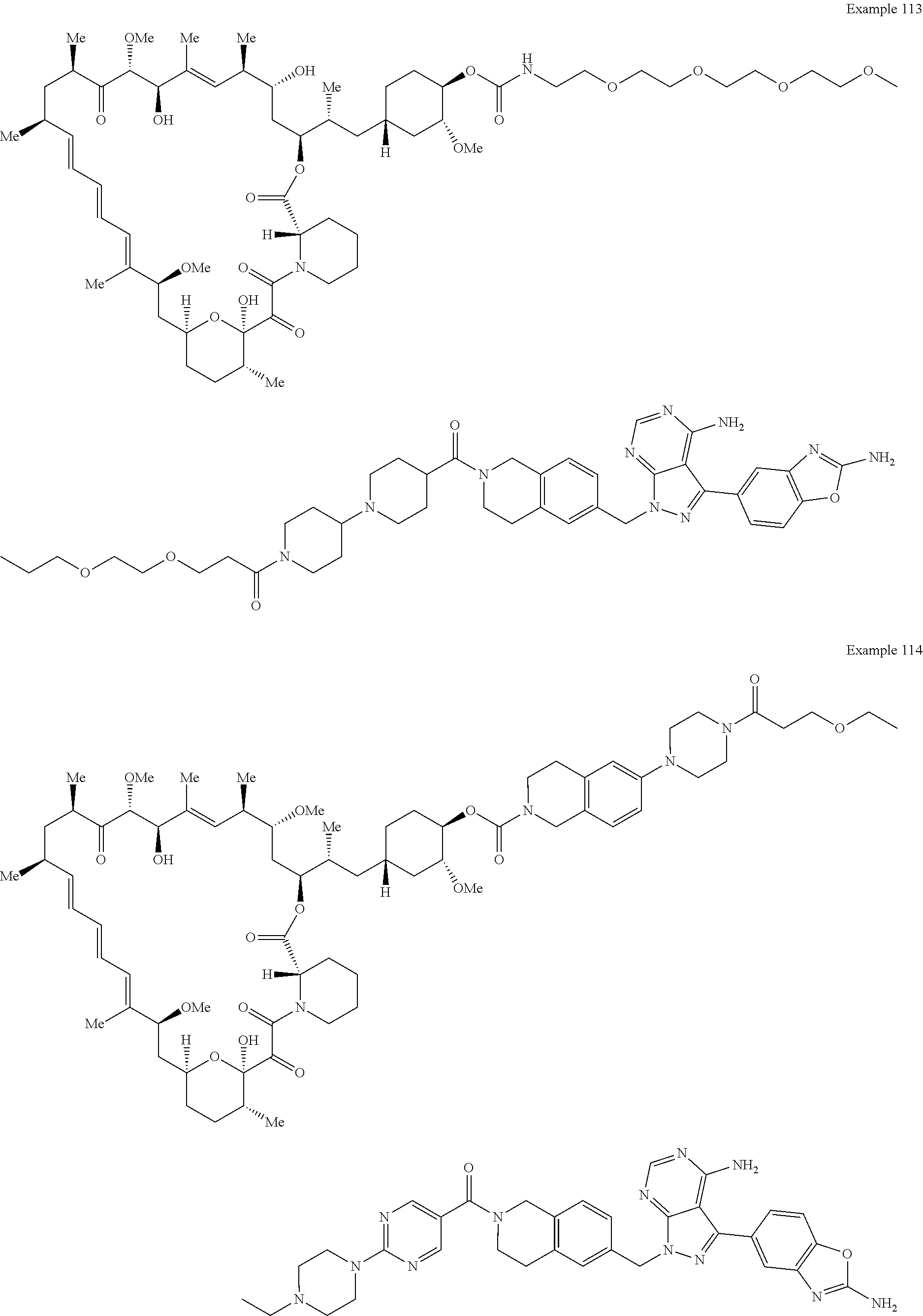

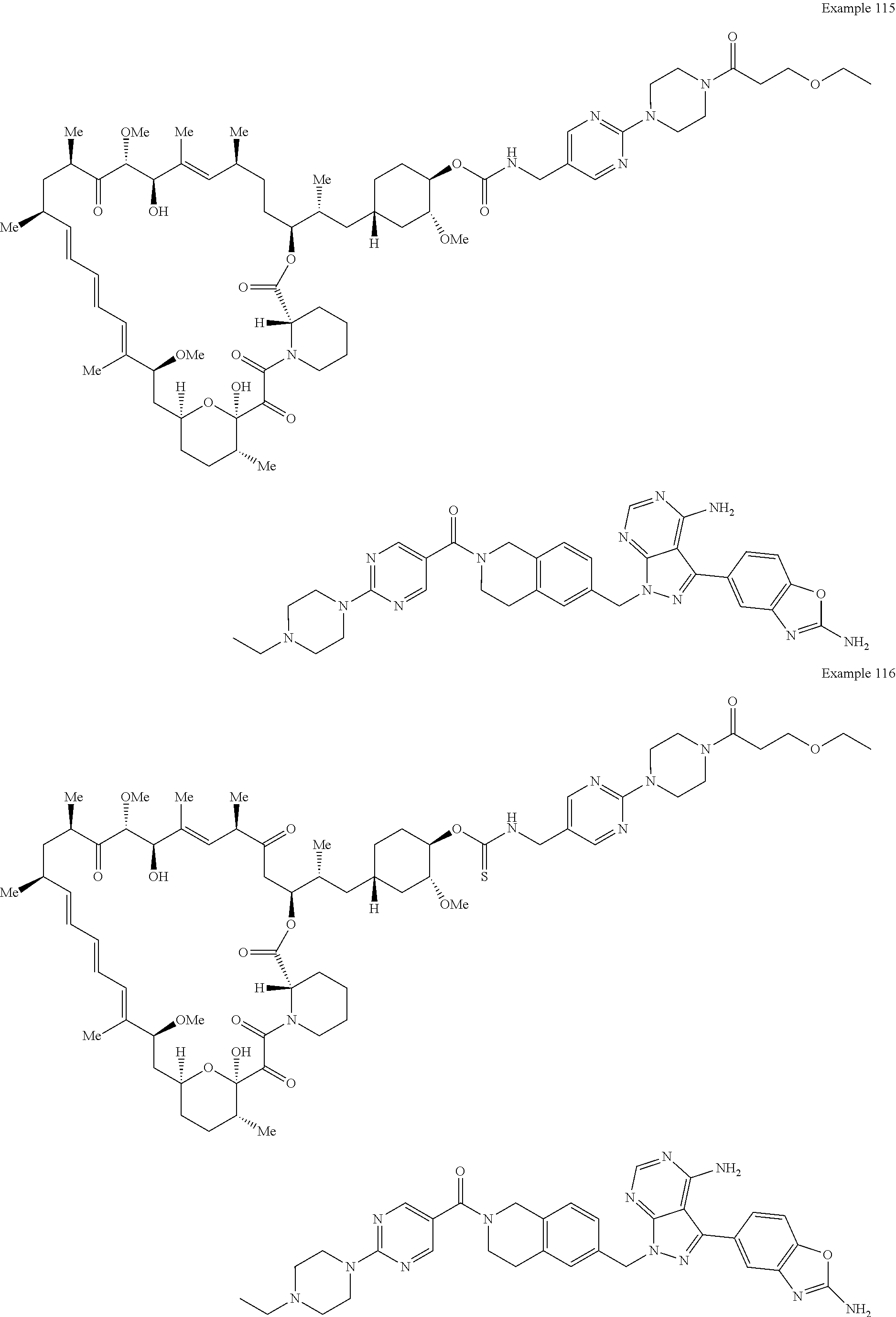

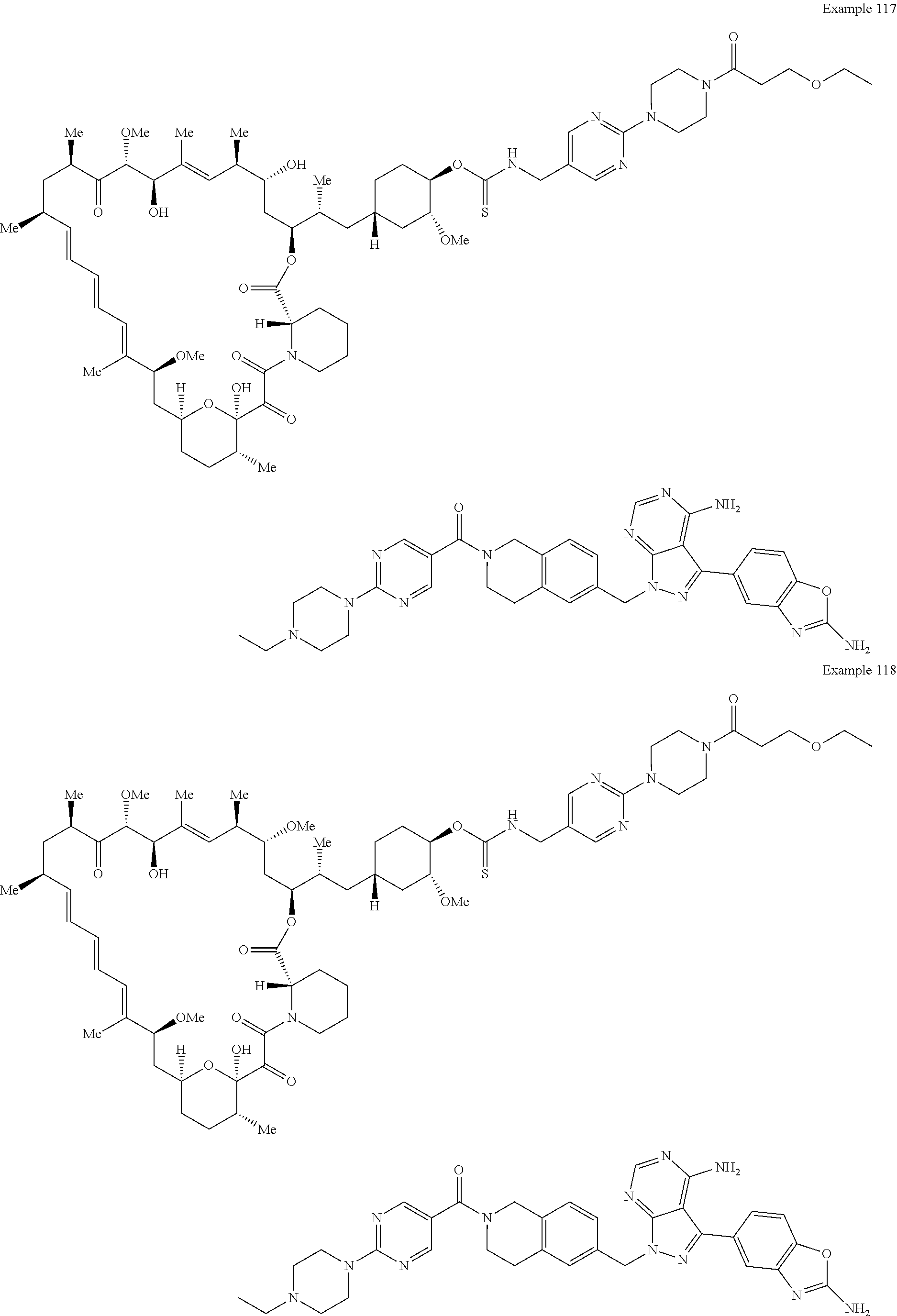

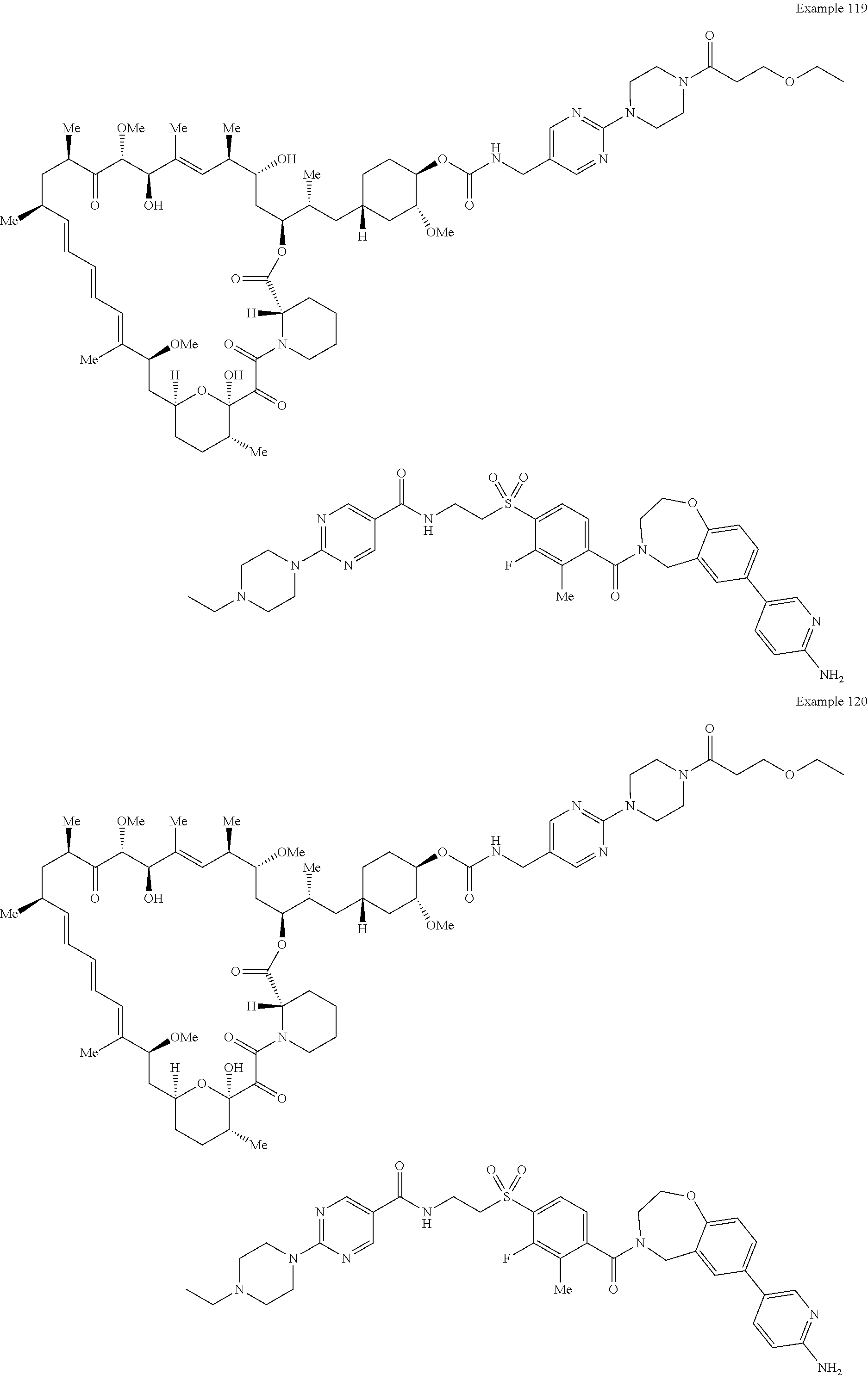

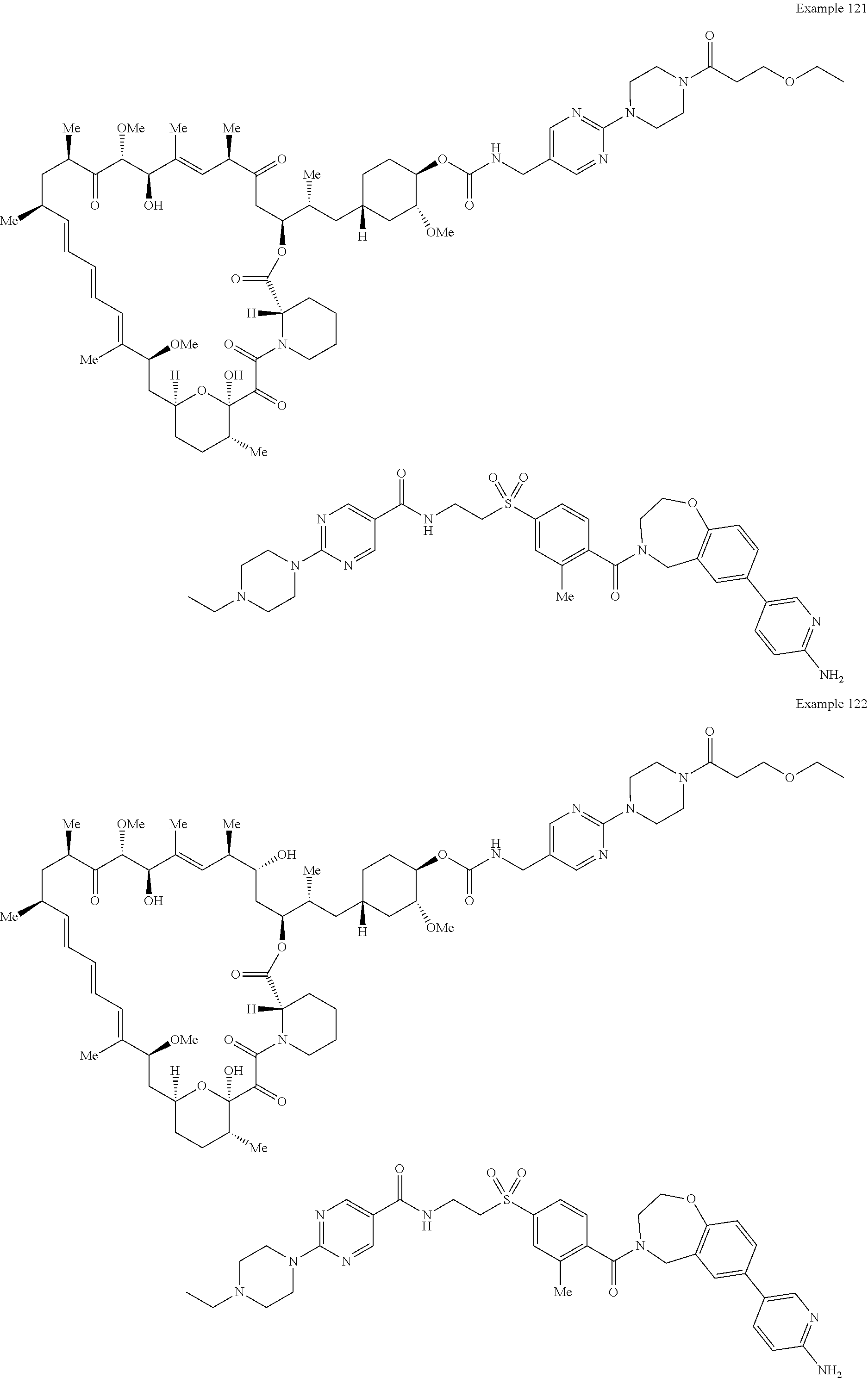

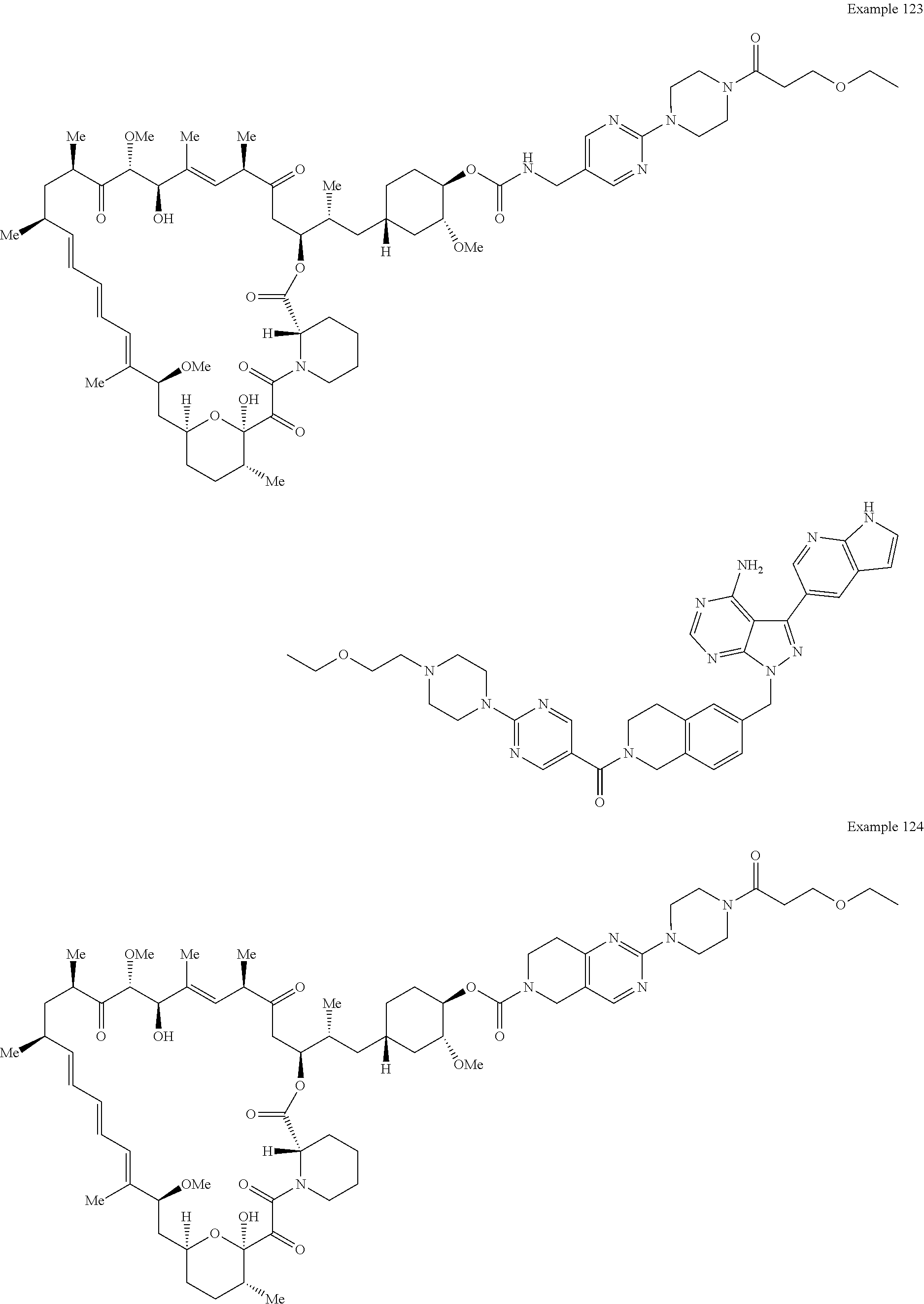

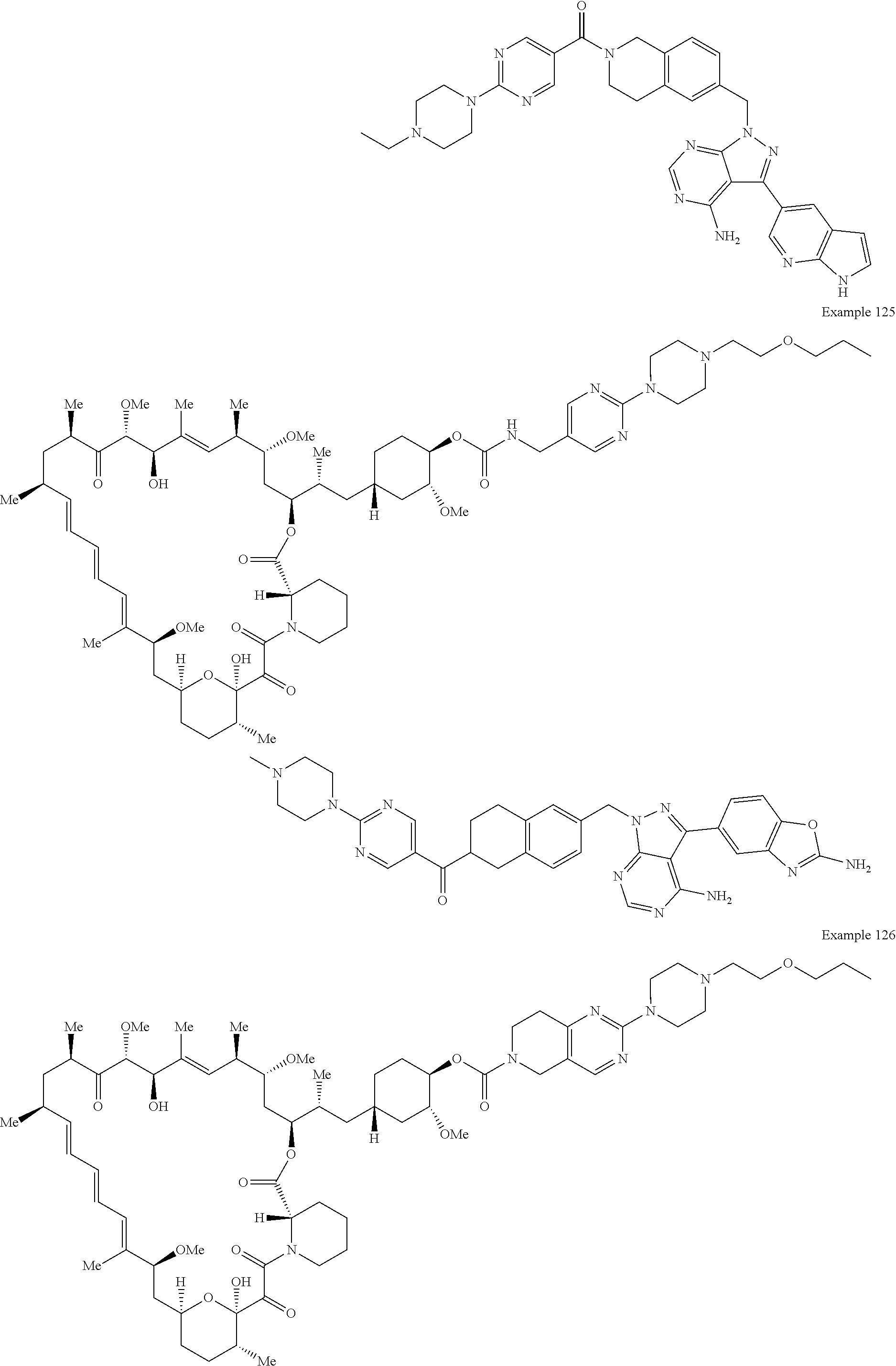

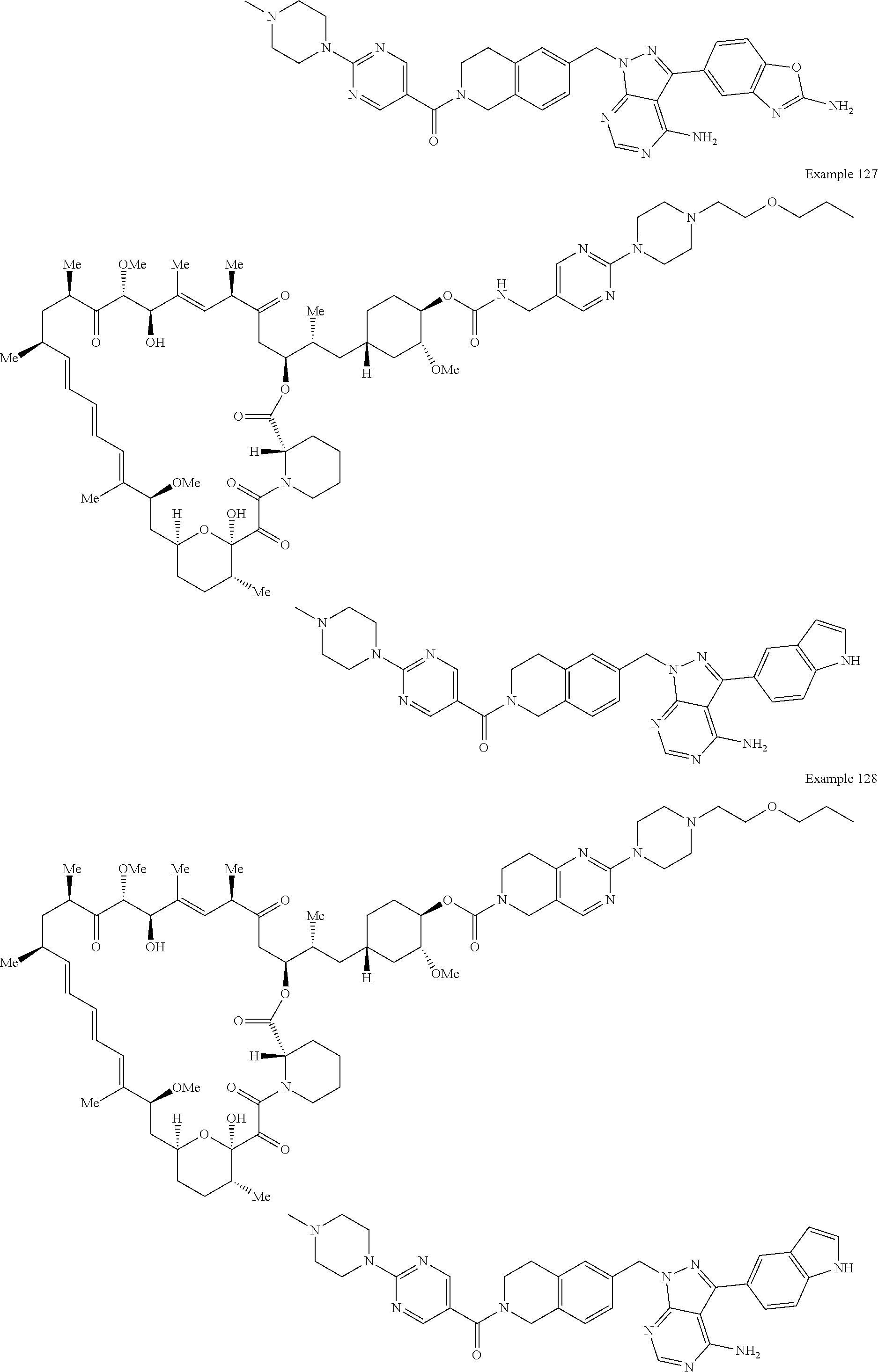

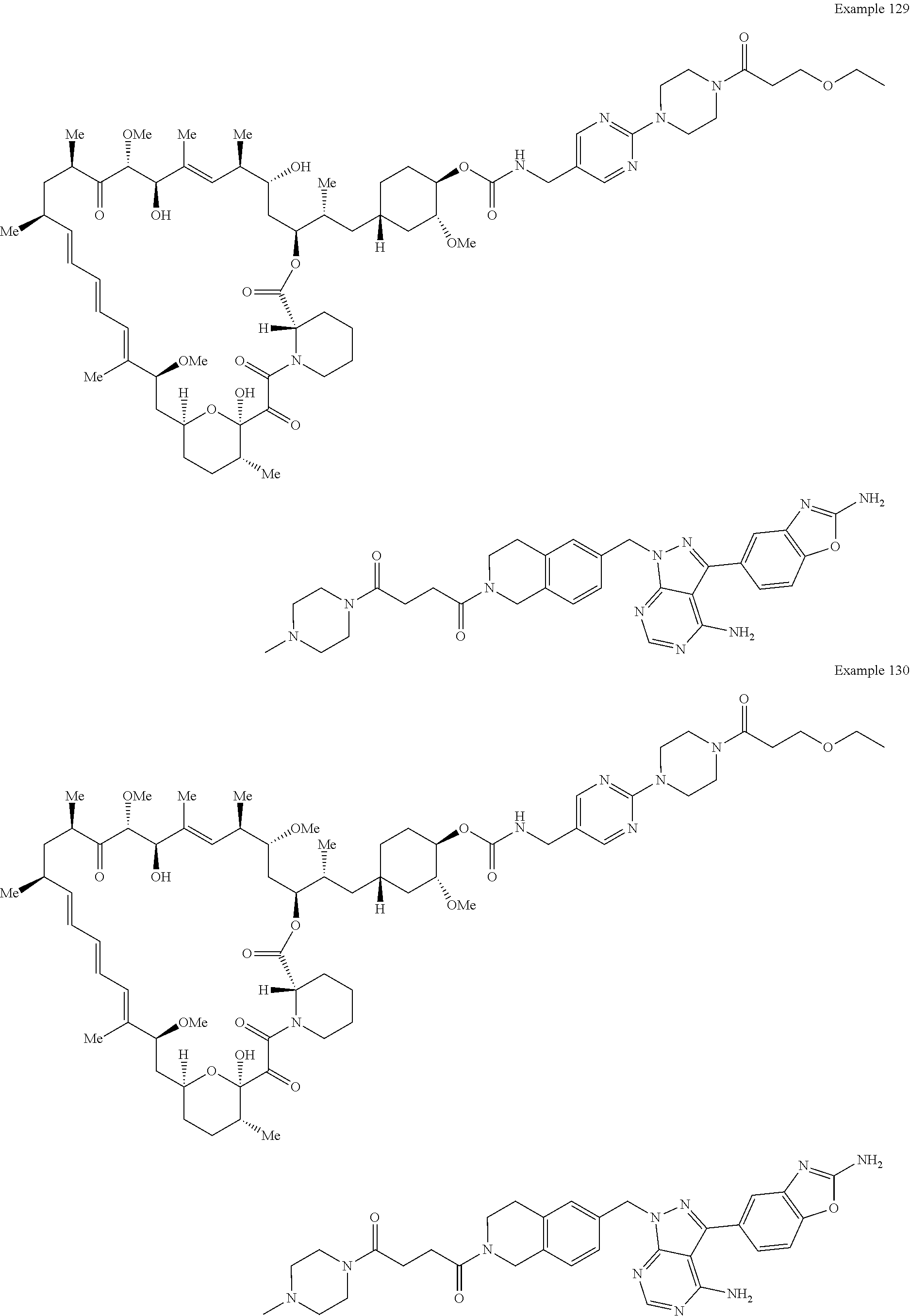

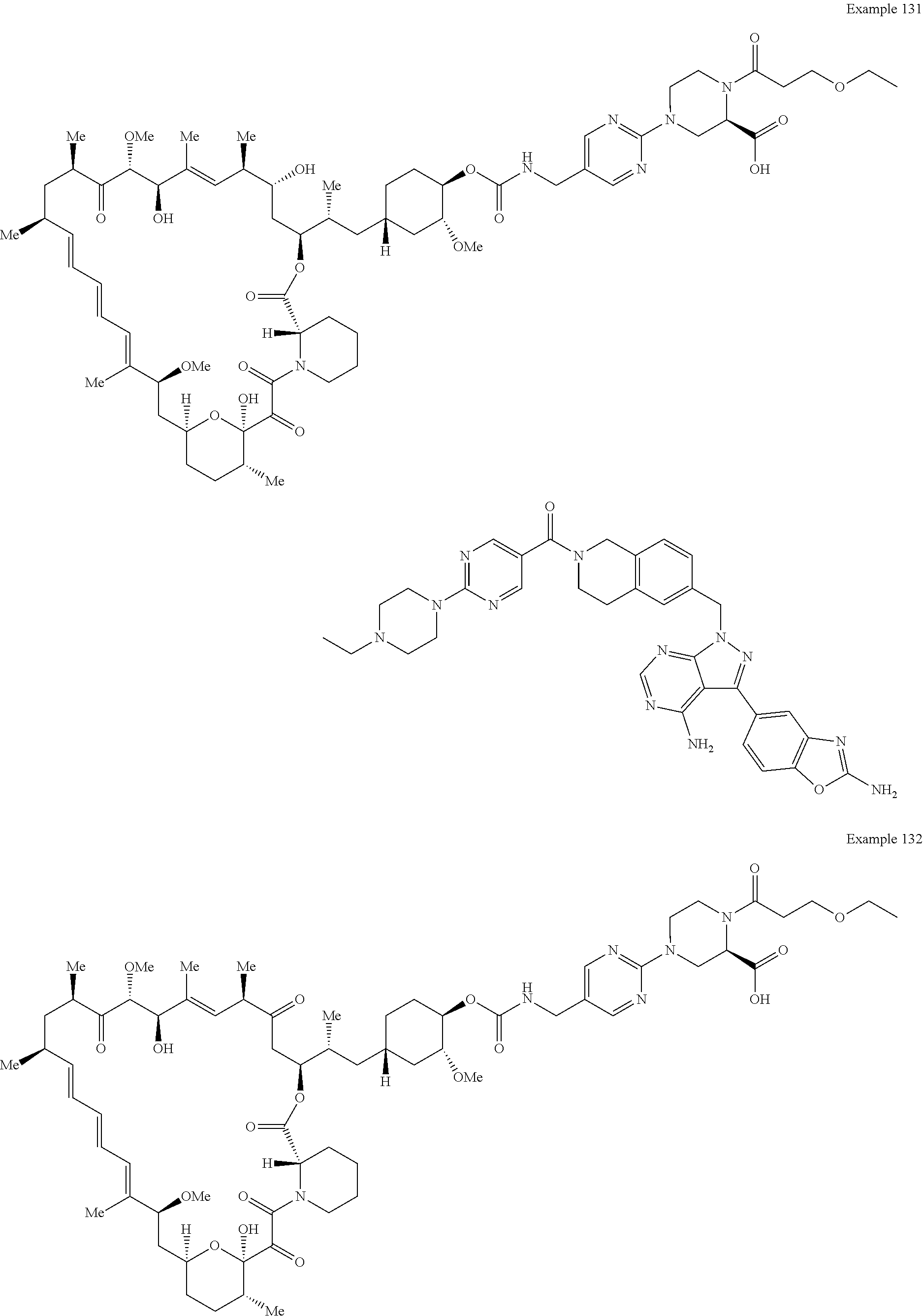

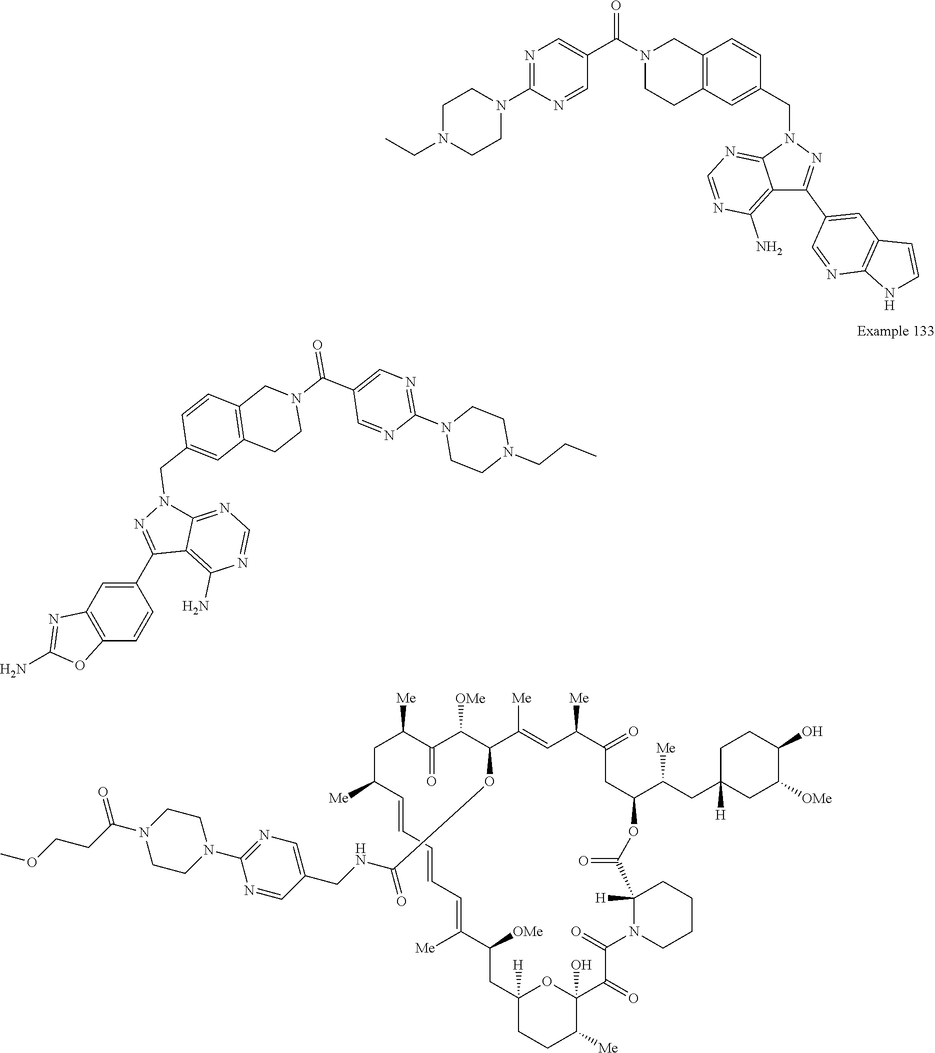

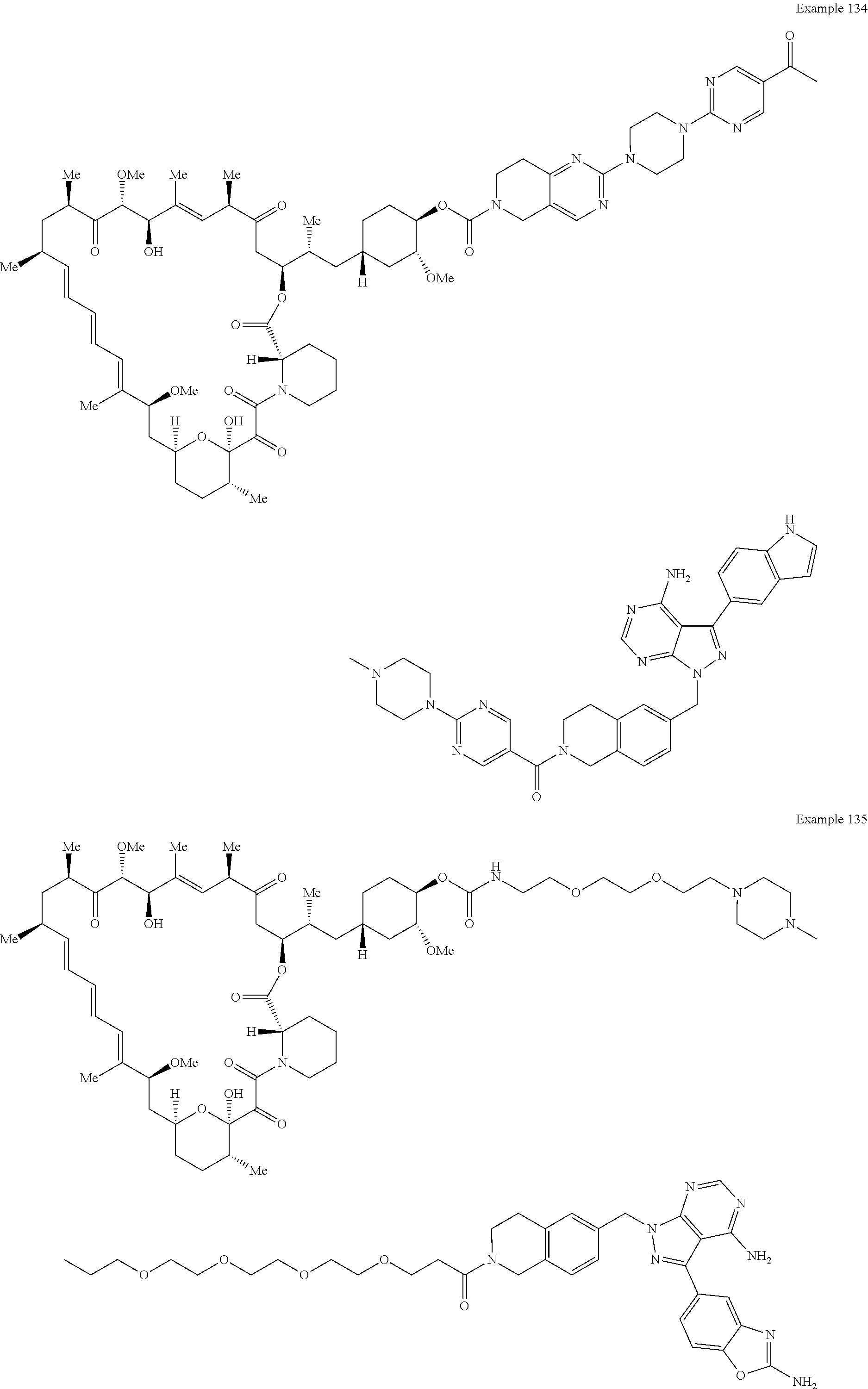

In certain embodiments, the present disclosure provides for a compound selected from below or a pharmaceutically acceptable salt or tautomer thereof,

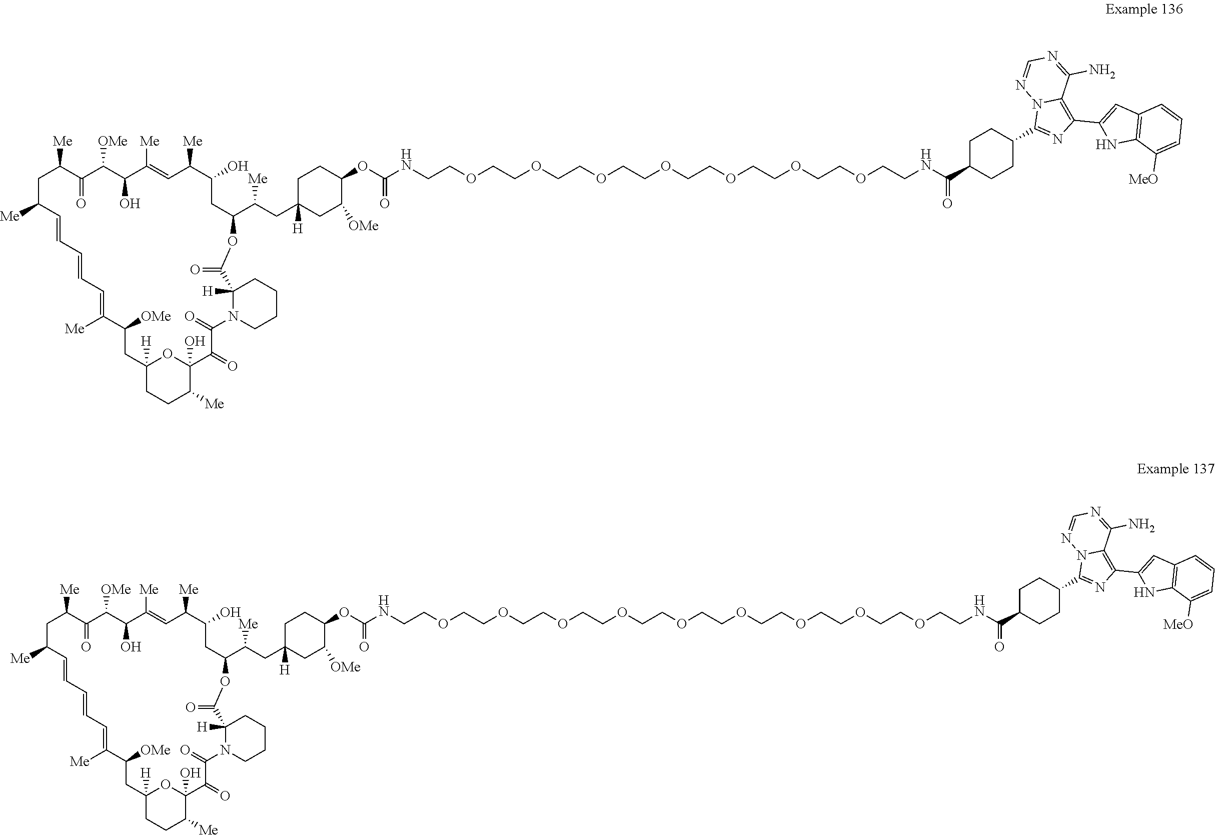

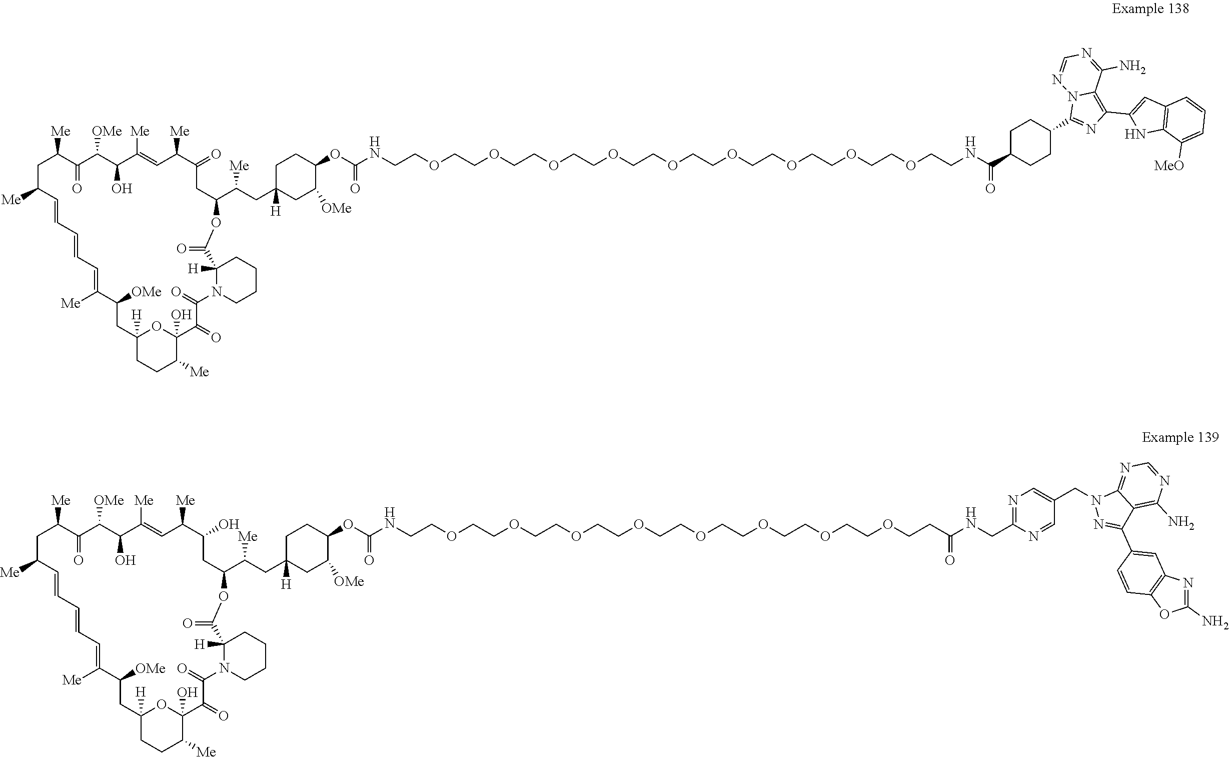

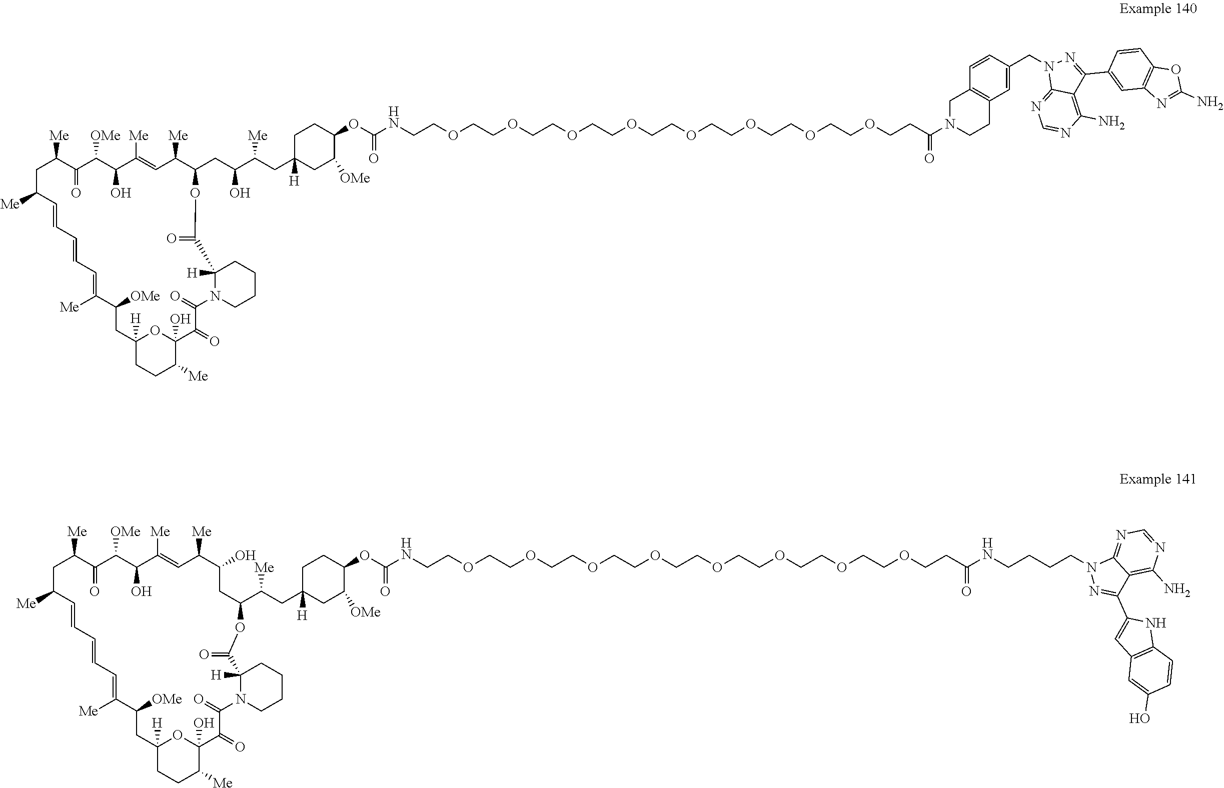

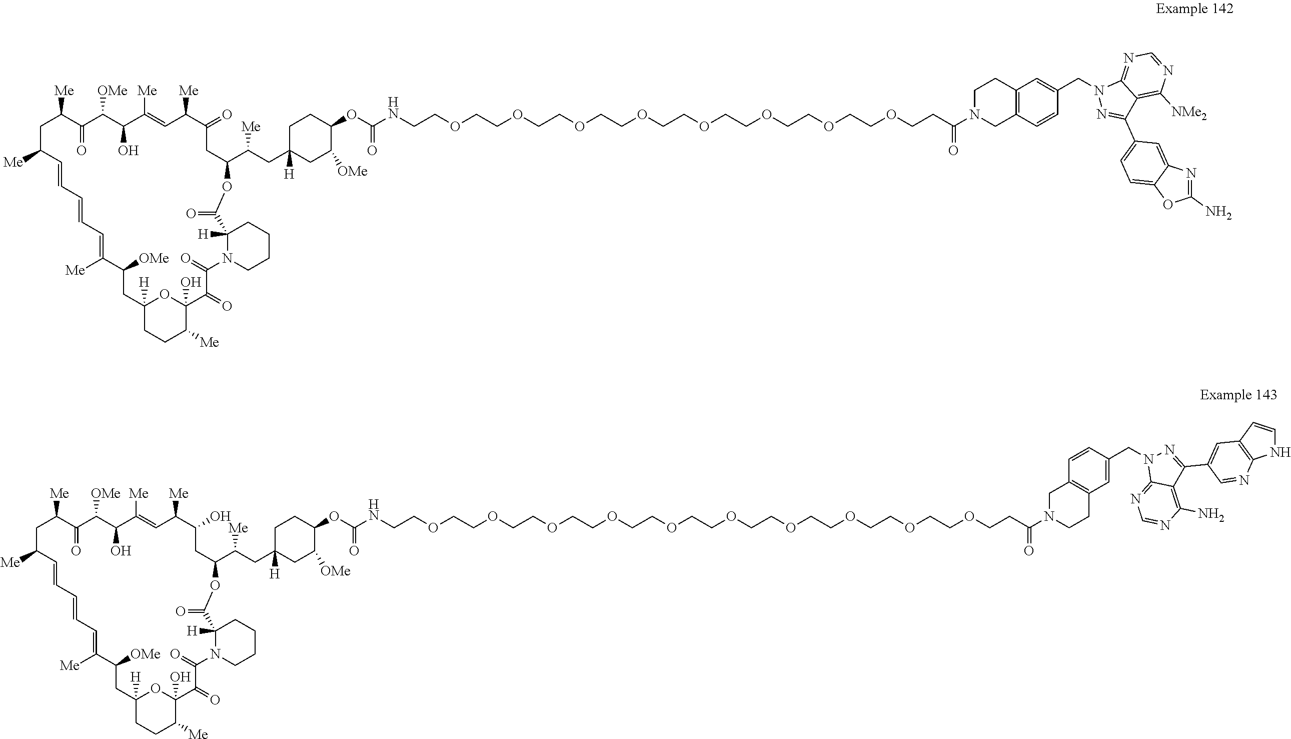

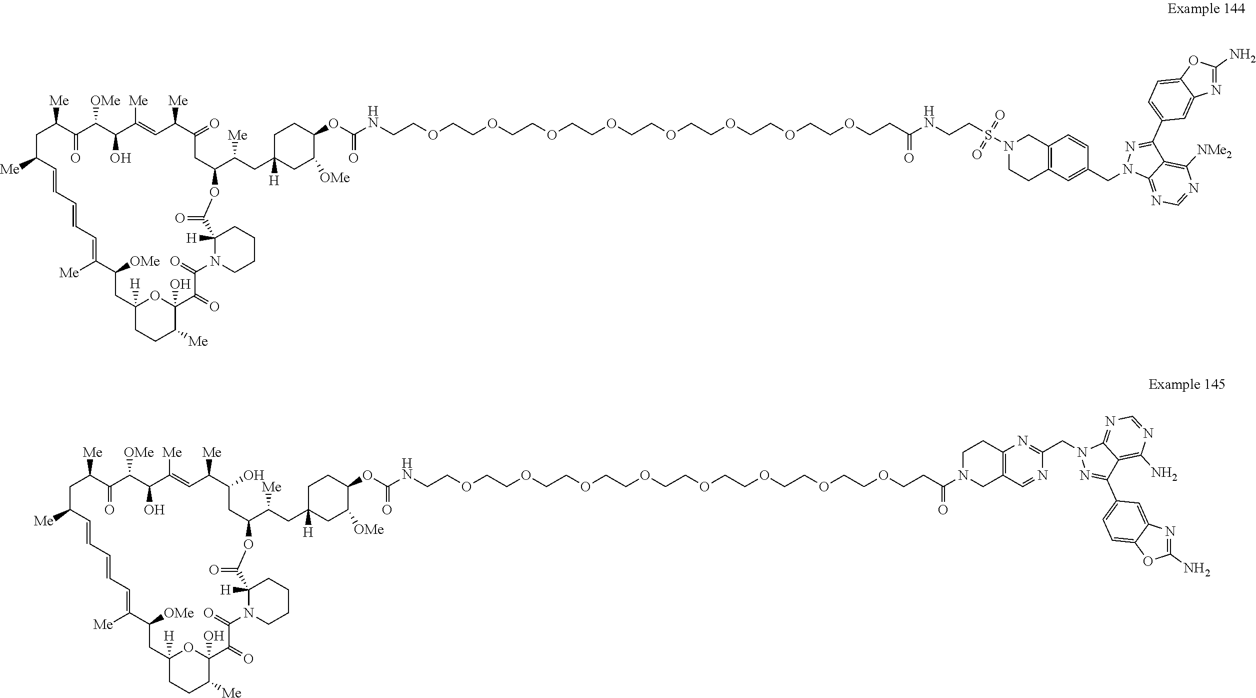

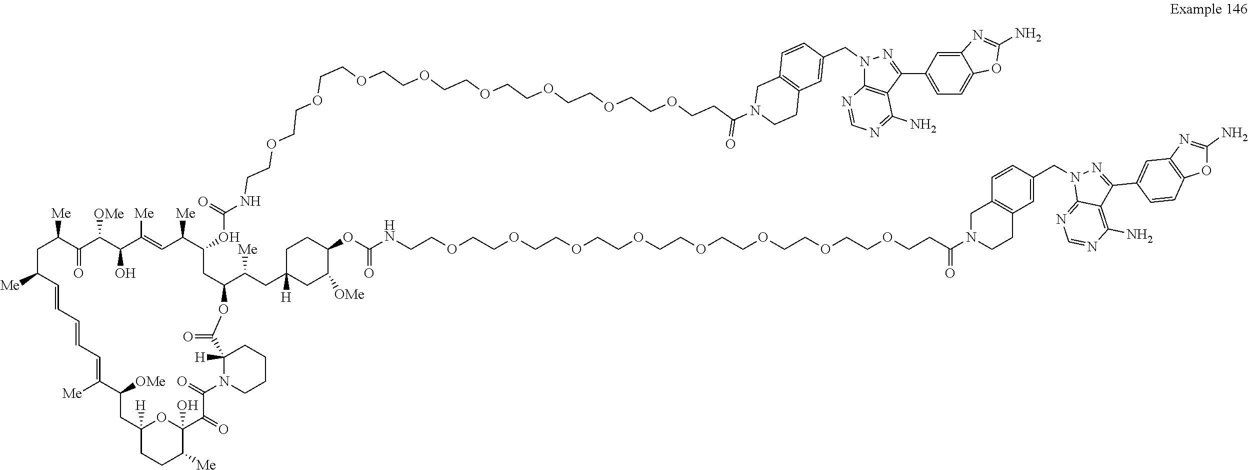 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@H](C)/C=C(\C)[C@@H](O)[C@@H](OC)C(=O)[C@H](C)C[C@H](C)/C=C/C=C/C=C/1C]

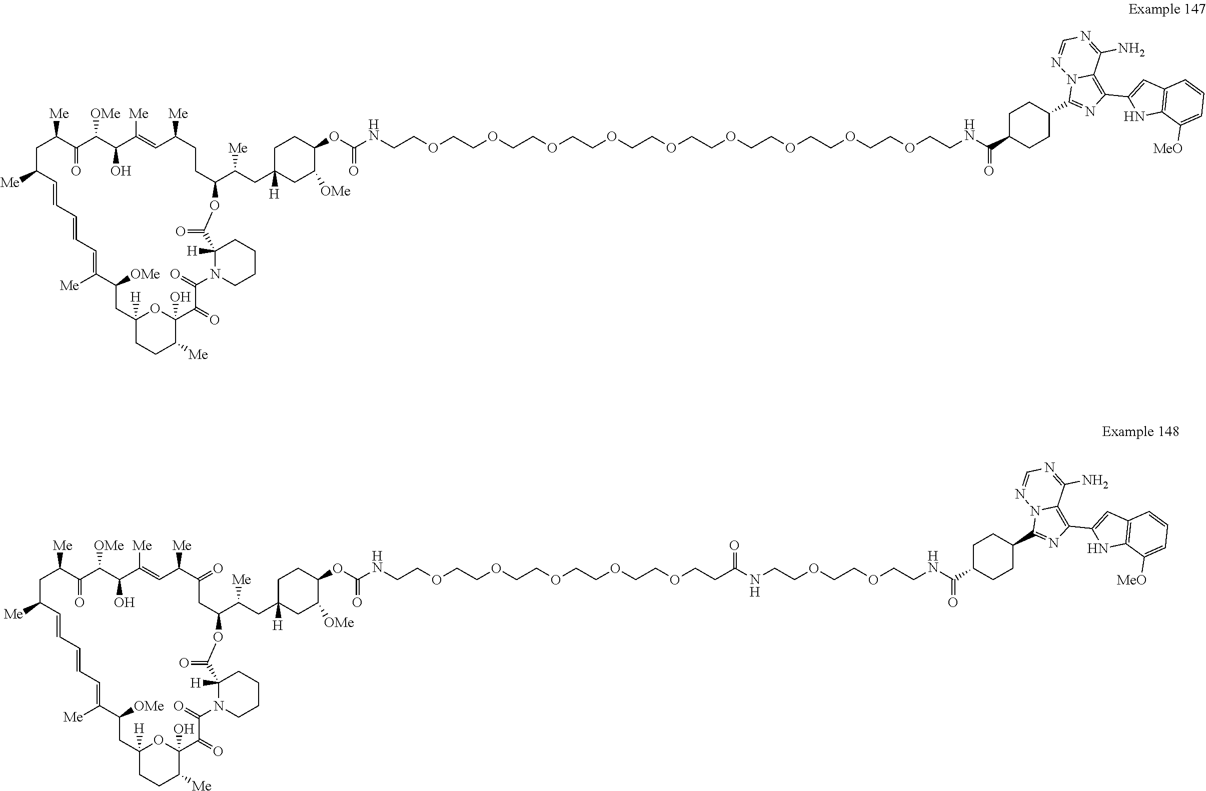

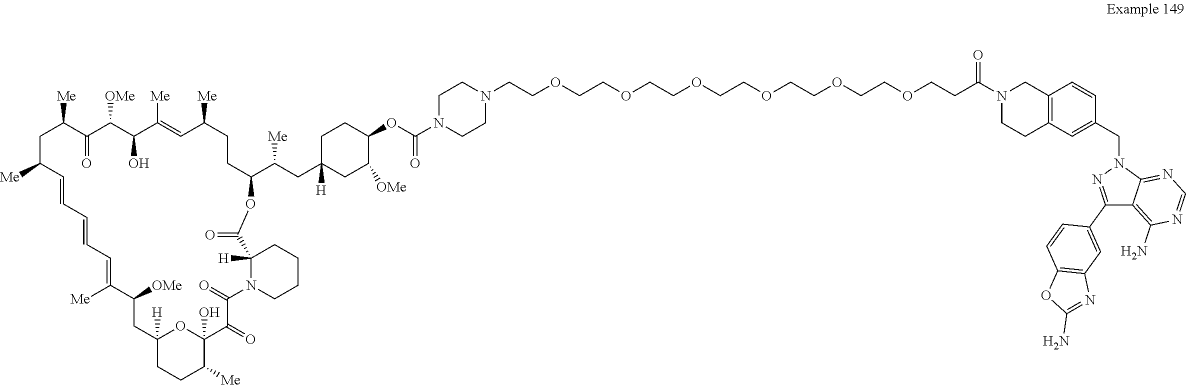 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(CCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)CC[C@H](C)/C=C(\C)[C@@H](O)[C@@H](OC)C(=O)[C@H](C)C[C@H](C)/C=C/C=C/C=C/1C]

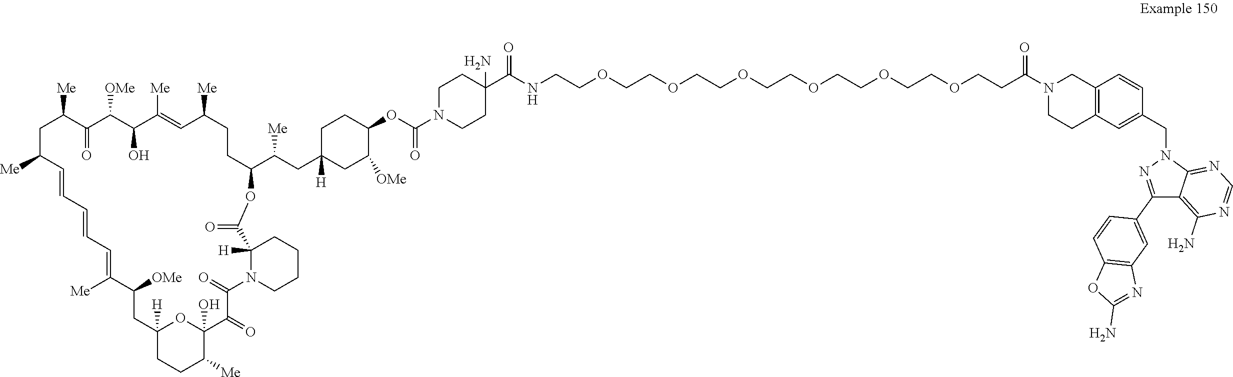 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCC(N)(C(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)CC[C@H](C)/C=C(\C)[C@@H](O)[C@@H](OC)C(=O)[C@H](C)C[C@H](C)/C=C/C=C/C=C/1C]

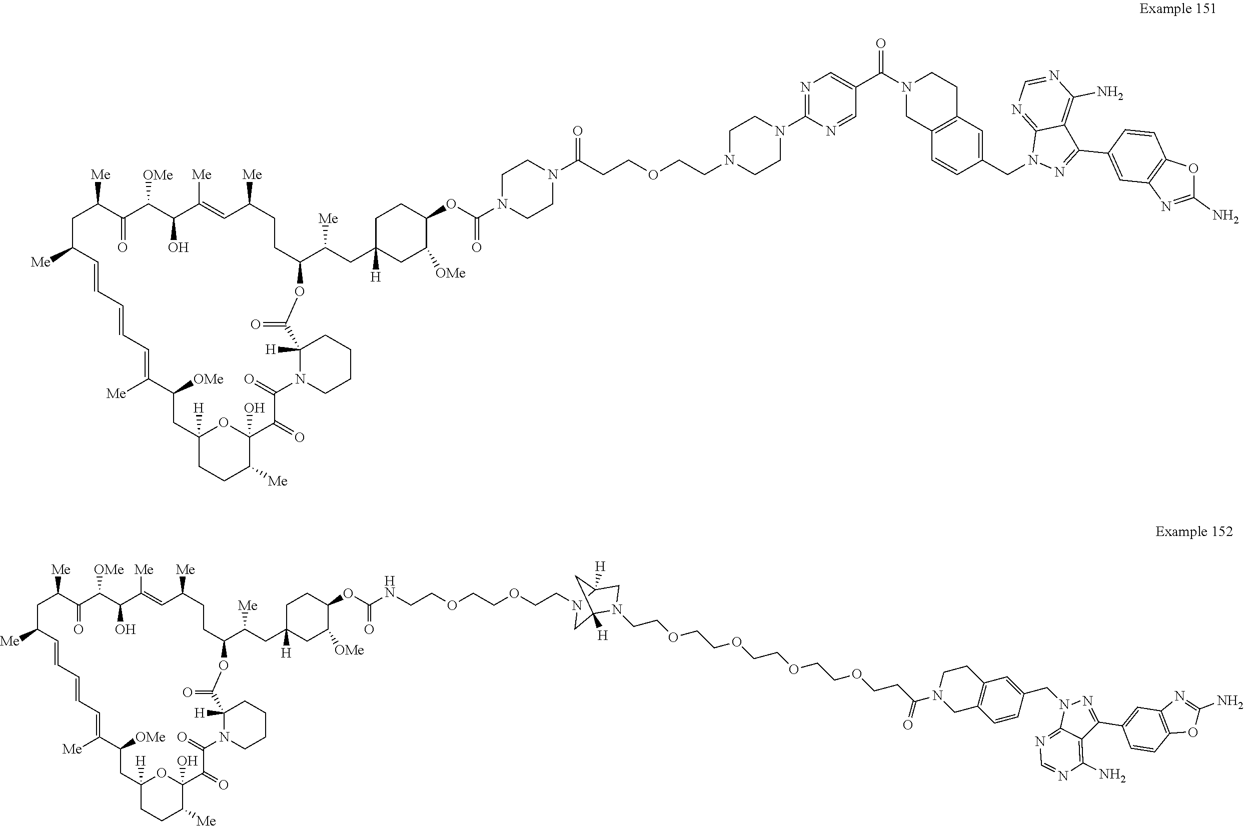

The compounds of the disclosure may include pharmaceutically acceptable salts of the compounds disclosed herein. Representative “pharmaceutically acceptable salts” may include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt. 3-hydroxy-2-naphthoate, oleate, oxalate, palmitate, pamoate, 1,1-methene-bis-2-hydroxy-3-naphthoate, einbonate, pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Pharmaceutically acceptable salt” may also include both acid and base addition salts. “Pharmaceutically acceptable acid addition salt” may refer to those salts which retain the biological effectiveness and properties of the free bases, which are not biologically or otherwise undesirable, and which may be formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” may refer to those salts that retain the biological effectiveness and properties of the free acids, which are not biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. For example, inorganic salts may include, but are not limited to, ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

Unless otherwise stated, structures depicted herein may also include compounds which differ only in the presence of one or more isotopically enriched atoms. For example, compounds having the present structure except for the replacement of a hydrogen atom by deuterium or tritium, or the replacement of a carbon atom by ¹³C or ¹⁴C, or the replacement of a nitrogen atom by ¹⁵N, or the replacement of an oxygen atom with ¹⁷O or ¹⁸O are within the scope of the disclosure. Such isotopically labeled compounds are useful as research or diagnostic tools.

In some embodiments, one or more deuterium atoms may be introduced into the PEG moiety of any compound of the present invention. Mechanisms for such modifications are known in the art starting from commercially available starting materials, such as isotopically enriched hydroxylamine building blocks. In some embodiments, a tritium or a deuterium may be introduced at the C32 position of compounds of the present invention using, for example, a commercially available isotopically pure reducing agent and methods known to those in the art. In some embodiments, ¹⁴C may be introduced into the C40 carbamate moiety of compounds of the present invention using commercially available materials and methods known to those of skill in the art. In some embodiments, an isotope such as deuterium or tritium may be introduced into the R^{40a} substituent of a compound of Formula Ia, Ic, I or II, using commercially available starting materials and methods known to those of skill in the art.

### Methods of Synthesizing Disclosed Compounds

The compounds of the present disclosure may be made by a variety of methods, including standard chemistry. Suitable synthetic routes are depicted in the schemes given below.

The compounds of any of the formulae described herein may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts. “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula I, Ia, Ib, II, or IIb, or a pharmaceutically acceptable salt or tautomer of any of the foregoing.

The compounds of any of the formulae described herein may be prepared by methods which avoid the use of metal-mediated cycloaddition reactions which require the use of azide-containing compounds. Azide containing compounds present potential safety hazards associated with their preparation and storage (e.g., explosion due to high energy decomposition). Also, the reaction schemes herein can avoid the use of copper or ruthenium metals in the penultimate or ultimate synthetic steps, which can be advantageous. Avoiding the use of copper or ruthenium metals in the penultimate or ultimate synthetic steps reduces the potential for contamination of the final compounds with undesirable metal impurities.

As rapamycin can be an expensive starting material, good yields on reactions are advantageous. The reaction schemes herein provide better yields than other reaction schemes. In the reaction schemes herein, there is no need to alkylate at the C40-hydroxyl of rapamycin, which is advantageous for providing as much as a 5-fold improved overall yield in preparing bivalent compounds from rapamycin compared to other reaction schemes.

There is an additional synthetic improvement associated with better yields. Avoiding the need to alkylate at the C40-hydroxyl gives as much as a 5-fold improved overall yield in preparing bivalent compounds from rapamycin.

Those skilled in the art will recognize if a stereocenter exists in any of the compounds of the present disclosure. Accordingly, the present disclosure may include both possible stereoisomers (unless specified in the synthesis) and may include not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be effected by any suitable method known in the art. See, for example. “Stereochemistry of Organic Compounds” by E. L. Eliel, S. H. Wilen, and L. N. Mander (Wiley-Interscience, 1994).

### Preparation of Compounds

The compounds described herein may be made from commercially available starting materials or synthesized using known organic, inorganic, and/or enzymatic processes.

The compounds of the present disclosure can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods may include but are not limited to those methods described below.

The term “tautomers” may refer to a set of compounds that have the same number and type of atoms, but differ in bond connectivity and are in equilibrium with one another. A “tautomer” is a single member of this set of compounds. Typically a single tautomer is drawn but it may be understood that this single structure may represent all possible tautomers that might exist. Examples may include enol-ketone tautomerism. When a ketone is drawn it may be understood that both the enol and ketone forms are part of the disclosure.

In addition to tautomers that may exist at all amide, carbonyl, and oxime groups within compounds of Formula I, Ia, Ib, Ic, II, or IIb, compounds in this family readily interconvert via a ring-opened species between two major isomeric forms, known as the pyran and oxepane isomers (shown below). This interconversion can be promoted by magnesium ions, mildly acidic conditions, or alkylamine salts, as described in the following references: i) Hughes, P. F.; Musser, J.; Conklin, M.; Russo. R. 1992. Tetrahedron Lett. 33(33): 4739-32. ii) Zhu, T. 2007. U.S. Pat. No. 7,241,771; Wyeth. iii) Hughes, P. F. 1994. U.S. Pat. No. 5,344,833; American Home Products Corp. The scheme below shows an interconversion between the pyran and oxepane isomers in compounds of Formula I, Ia, Ib, Ic, II, or IIb.

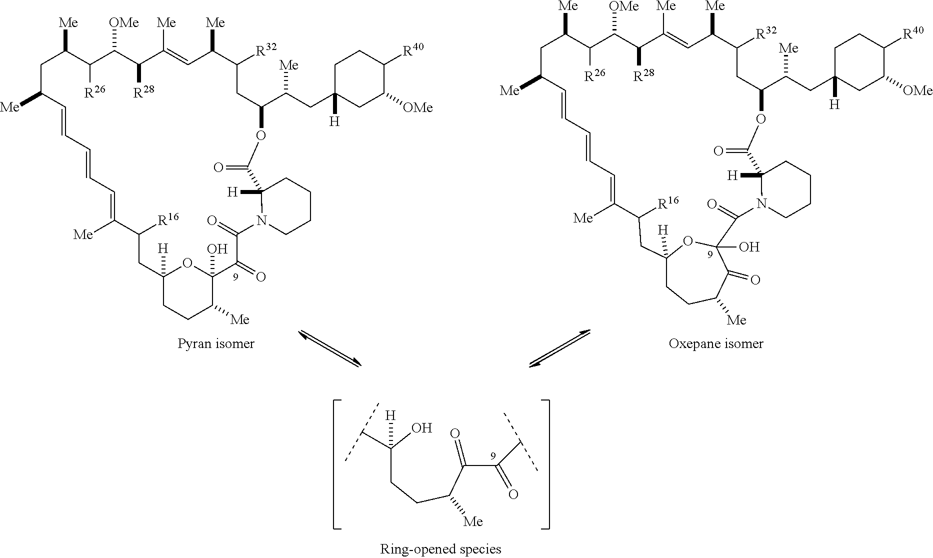

As this interconversion occurs under mild condition, and the thermodynamic equilibrium position may vary between different members of compounds of Formula I, Ia, Ib, Ic, II, or IIb, both isomers are contemplated for the compounds of Formula I, Ia, Ib, Ic, II, or IIb. For the sake of brevity, the pyran isomer form of all intermediates and compounds of Formula I, Ia, Ib, Ic, II, or IIb is shown.

### General Assembly Approaches for Bifunctional Rapalogs

With reference to the schemes below, rapamycin is Formula RAP.

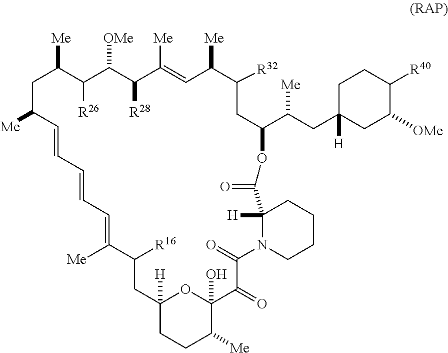

where R¹⁶ is —OCH₃; R²⁶ is ═O; R²⁸ is —OH; R³² is ═O; and R⁴⁰ is —OH. A “rapalog” refers to an analog or derivative of rapamycin. For example, with reference to the schemes below, a rapalog can be rapamycin that is substituted at any position, such as R¹⁶, R²⁶, R²⁸, R³², or R⁴⁰. An active site inhibitor (AS inhibitor) is an active site mTOR inhibitor. In certain embodiments, AS inhibitor is depicted by B, in Formula I, Ia, Ib, Ic, II, or IIb.

### Series 1 Bifunctional Rapalogs

A general structure of Series 1 bifunctional rapalogs is shown in Scheme 1 below. For these types of bifunctional rapalogs, the linker may include variations where q=0 to 30, such as q=1 to 7, and r=1 to 6. The linker amine can include substitutions, such as R═H and C₁-C₆ alkyl groups. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

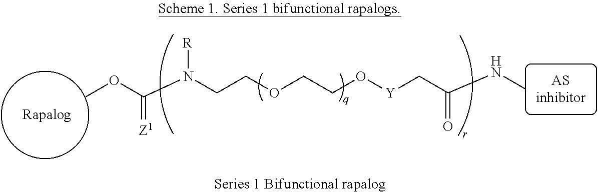

### Series 2 Bifunctional Rapalogs

A general structure of Series 2 bifunctional rapalogs is shown in Scheme 2 below. For these types of bifunctional rapalogs, the linker may include variations where q=0 to 30, such as q=1 to 7. The linker amine can include substitutions, such as R═H and C1-C6 alkyl groups. The pre-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

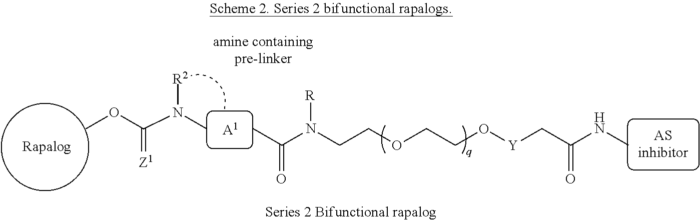

### Series 2 Bifunctional Rapalog

A general structure of Series 3 bifunctional rapalogs is shown in Scheme 3 below. For these types of bifunctional rapalogs, the linker may include variations where q=0 to 30, such as q=1 to 7. The linker amine can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═C or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

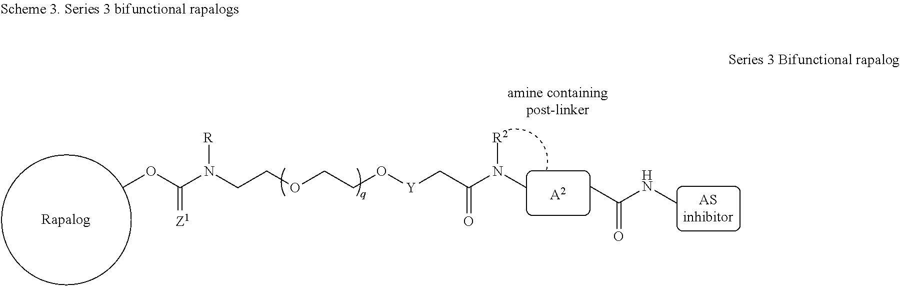

### Series 4 Bifunctional Rapalogs

A general structure of Series 4 bifunctional rapalogs is shown in Scheme 4 below. For these types of bifunctional rapalogs, the linker may include variations where q=0 to 30, such as q=1 to 7. The linker amine can include substitutions, such as R═H and C1-C6 alkyl groups. The pre- and post-linker amines can each include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

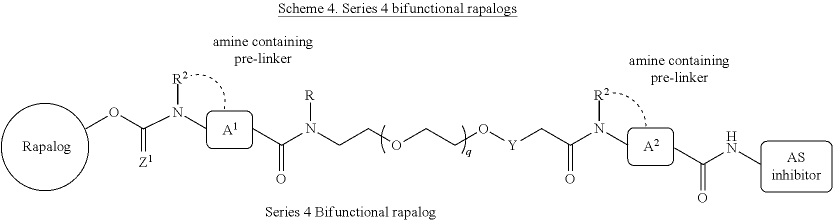

### Series 5 Bifunctional Rapalogs

A general structure of Series 5 bifunctional rapalogs is shown in Scheme 5 below. For these types of bifunctional rapalogs, the pre-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

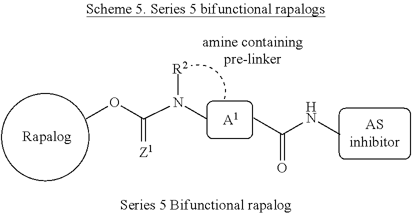

### Series 6 Bifunctional Rapalogs

A general structure of Series 6 bifunctional rapalogs is shown in Scheme 6 below. For these types of bifunctional rapalogs, the linker may include variations where q=0 to 30, such as q=1 to 7. The linker amines can include substitutions, such as R═H and C1-C6 alkyl groups. The post-linker amine can include substitutions, such as R²═H, C₁-C6 alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

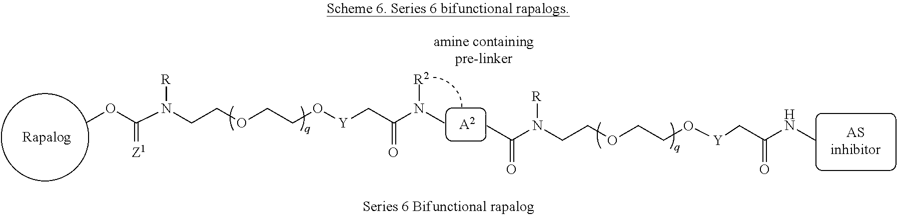

### Series 7 Bifunctional Rapalogs

A general structure of Series 7 bifunctional rapalogs is shown in Scheme 7 below. For these types of bifunctional rapalogs, the linker may include variations where q=0 to 30, such as q=1 to 7. The linker amine can include substitutions, such as R═H and C1-C6 alkyl groups. The pre- and post-linker amines can each include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

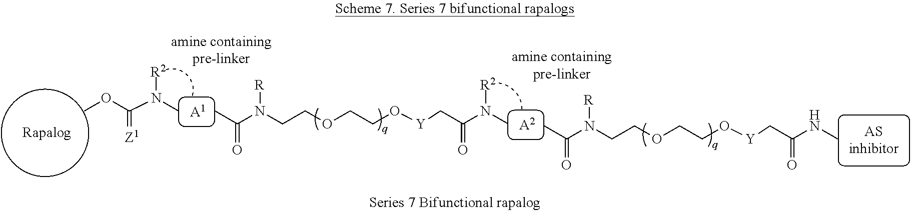

### Series 8 Bifunctional Rapalogs

A general structure of Series 8 bifunctional rapalogs is shown in Scheme 8 below. For these types of bifunctional rapalogs, the linker may include variations where q=0 to 30, such as q=1 to 7. The linker amine can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

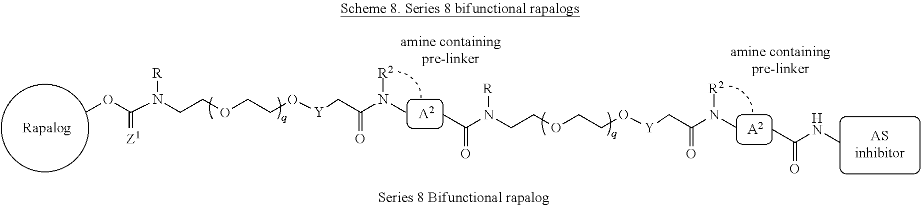

### Pharmaceutical Compositions

Another aspect provides a pharmaceutical composition including a pharmaceutically acceptable excipient and a compound of the present invention, or pharmaceutically acceptable salt or tautomer thereof.

In embodiments of the pharmaceutical compositions, a compound of the present invention, or a pharmaceutically acceptable salt or tautomer thereof, may be included in a therapeutically effective amount.

Administration of the disclosed compounds or compositions can be accomplished via any mode of administration for therapeutic agents. These modes may include systemic or local administration such as oral, nasal, parenteral, transdermal, subcutaneous, vaginal, buccal, rectal, topical, intrathecal, or intracranial administration modes.

In certain embodiments, administering can include oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration can be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Set Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn. Curr. Opin. Biotechnol. 6:698-708, 1995; Ostro, Am. J. Hosp. Pharm. 46: 1576-1587, 1989). The compositions of the present disclosure can also be delivered as nanoparticles.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, intrathecal, subcutaneous or intramuscular form, and all using forms well known to those skilled in the pharmaceutical arts.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin. PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure relates to a pharmaceutical composition comprising a compound, or a pharmaceutically acceptable salt or tautomer thereof, of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

mTOR and Methods of Treatment

The term “mTOR” refers to the protein “mechanistic target of rapamycin (serine/threonine kinase)” or “mammalian target of rapamycin.” The term “mTOR” may include both the wild-type form of the nucleotide sequences or proteins as well as any mutants thereof. In some embodiments, “mTOR” is wild-type mTOR. In some embodiments, “mTOR” is one or more mutant forms. The term “mTOR” XYZ may refer to a nucleotide sequence or protein of a mutant mTOR wherein the Y numbered amino acid of mTOR that normally has an X amino acid in the wildtype, instead has a Z amino acid in the mutant. In embodiments, an mTOR is the human mTOR.

The term “mTORC1” refers to the protein complex including mTOR and Raptor (regulatory-associated protein of mTOR). mTORC1 may also include MLST8 (mammalian lethal with SEC 13 protein 8), PRAS40, and/or DEPTOR. mTORC1 may function as a nutrient/energy/redox sensor and regulator of protein synthesis. The term “mTORC1 pathway” or “mTORC1 signal transduction pathway” may refer to a cellular pathway including mTORC1. An mTORC1 pathway includes the pathway components upstream and downstream from mTORC1. An mTORC1 pathway is a signaling pathway that is modulated by modulation of mTORC1 activity. In embodiments, an mTORC1 pathway is a signaling pathway that is modulated by modulation of mTORC1 activity but not by modulation of mTORC2 activity. In embodiments, an mTORC1 pathway is a signaling pathway that is modulated to a greater extent by modulation of mTORC1 activity than by modulation of mTORC2 activity.

The term “mTORC2” refers to the protein complex including mTOR and RICTOR (rapamycin-insensitive companion of mTOR). mTORC2 may also include GβL, mSIN1 (mammalian stress-activated protein kinase interacting protein 1). Protor 1/2. DEPTOR, TTI1, and/or TEL2. mTORC2 may regulate cellular metabolism and the cytoskeleton. The term “mTORC2 pathway” or “mTORC2 signal transduction pathway” may refer to a cellular pathway including mTORC2. An mTORC2 pathway includes the pathway components upstream and downstream from mTORC2. An mTORC2 pathway is a signaling pathway that is modulated by modulation of mTORC2 activity. In embodiments, an mTORC2 pathway is a signaling pathway that is modulated by modulation of mTORC2 activity but not by modulation of mTORC1 activity. In embodiments, an mTORC2 pathway is a signaling pathway that is modulated to a greater extent by modulation of mTORC2 activity than by modulation of mTORC1 activity.

The term “rapamycin” or “sirolimus” refers to a macrolide produced by the bacteria Streptomyces hygroscopicus. Rapamycin may prevent the activation of T cells and B cells. Rapamycin has the IUPAC name (3S,6R,7E,9R, 10R, 12R, 14S, 15E, 17E, 19E,21S,23S,26R,27R,34aS)-9, 10, 12, 13, 14,21,22,23,24,25,26,27,32,33,34,34a-hexadecahydro-9,27-dihydroxy-3-[(1R)-2-[(1S,3 R,4R)-4-hydroxy-3-methoxycyclohexyl]-1-methylethyl]-10,21-dimethoxy-6,8, 12, 14,20,26-hexamethyl-23,27-epoxy-3H-pyrido[2, 1-c][1,4]-oxaazacyclohentriacontine-1,5, 11,28,29(4H,6H,31H)-pentone. Rapamycin has the CAS number 53123-88-9. Rapamycin may be produced synthetically (e.g., by chemical synthesis) or through use of a production method that does not include use of Streptomyces hygroscopicus.

“Analog” is used in accordance with its plain ordinary meaning within chemistry and biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof.

The term “rapamycin analog” or “rapalog” refers to an analog or derivative (e.g., a prodrug) of rapamycin.

The terms “active site mTOR inhibitor” and “ATP mimetic” refers to a compound that inhibits the activity of mTOR (e.g., kinase activity) and binds to the active site of mTOR (e.g., the ATP binding site, overlapping with the ATP binding site, blocking access by ATP to the ATP binding site of mTOR). Examples of active site mTOR inhibitors include, but are not limited to, ΓNK128, PP242, PP121, MLN0128, AZD8055, AZD2014, NVP-BEZ235, BGT226, SF1126, Torin 1. Torin 2, WYE 687, WYE 687 salt (e.g., hydrochloride), PF04691502, PI-103, CC-223, OSI-027, XL388, KU-0063794, GDC-0349, and PKI-587. In embodiments, an active site mTOR inhibitor is an asTORi. In some embodiments, “active site inhibitor” may refer to “active site mTOR inhibitor.”

The term “FKBP” refers to the protein Peptidyl-prolyl cis-trans isomerase. For non-limiting examples of FKBP, see Cell Mol Life Sci. 2013 September; 70(18):3243-75. In embodiments, “FKBP” may refer to “FKBP-12” or “FKBP 12” or “FKBP 1 A.” In embodiments. “FKBP” may refer to the human protein. Included in the term “FKBP” is the wildtype and mutant forms of the protein. In embodiments, “FKBP” may refer to the wildtype human protein. In embodiments, “FKBP” may refer to the wildtype human nucleic acid. In embodiments, the FKBP is a mutant FKBP. In embodiments, the mutant FKBP is associated with a disease that is not associated with wildtype FKBP. In embodiments, the FKBP includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype FKBP.

The term “FKBP-12” or “FKBP 12” or “FKBP1A” may refer to the protein “Peptidyl-prolyl cis-trans isomerase FKBP 1 A.” In embodiments. “FKBP-12” or “FKBP 12” or “FKBP 1 A” may refer to the human protein. Included in the term “FKBP-12” or “FKBP 12” or “FKBP 1 A” are the wildtype and mutant forms of the protein In embodiments, the reference numbers immediately above may refer to the protein, and associated nucleic acids, known as of the date of filing of this application. In embodiments, “FKBP-12” or “FKBP 12” or “FKBP 1 A” may refer to the wildtype human protein. In embodiments, “FKBP-12” or “FKBP 12” or “FKBP1A” may refer to the wildtype human nucleic acid. In embodiments, the FKBP-12 is a mutant FKBP-12. In embodiments, the mutant FKBP-12 is associated with a disease that is not associated with wildtype FKBP-12. In embodiments, the FKBP-12 may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype FKBP-12. In embodiments, the FKBP-12 has the protein sequence corresponding to reference number GI:206725550.

The term “4E-BP1” or “4EBP1” or “EIF4EBP1” refers to the protein “Eukaryotic translation initiation factor 4E-binding protein 1.” In embodiments, “4E-BP1” or “4EBP1” or “EIF4EBP1” may refer to the human protein. Included in the term “4E-BP 1” or “4EBP1” or “EIF4EBP1” are the wildtype and mutant forms of the protein. In embodiments, the reference numbers immediately above may refer to the protein, and associated nucleic acids, known as of the date of filing of this application. In embodiments, “4E-BP 1” or “4EBP1” or “EIF4EBP1” may refer to the wildtype human protein. In embodiments, “4E-BP1” or “4EBP1” or “EIF4EBP1” may refer to the wildtype human nucleic acid. In embodiments, the 4EBP1 is a mutant 4EBP1. In embodiments, the mutant 4EBP1 is associated with a disease that is not associated with wildtype 4EBP1. In embodiments, the 4EBP1 may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype 4EBP1. In embodiments, the 4EBP1 has the protein sequence corresponding to reference number GL4758258.

The term “Akt” refers to the serine/threonine specific protein kinase involved in cellular processes such as glucose metabolism, apoptosis, proliferation, and other functions, also known as “protein kinase B” (PKB) or “Akt1.” In embodiments, “Akt” or “AM” or “PKB” may refer to the human protein. Included in the term “Akt” or “Akt1” or “PKB” are the wildtype and mutant forms of the protein. In embodiments, the reference numbers immediately above may refer to the protein, and associated nucleic acids, known as of the date of filing of this application. In embodiments, “Akt” or “Akt1” or “PKB” may refer to the wildtype human protein. In embodiments, “Akt” or “Akt1” or “PKB” may refer to the wildtype human nucleic acid. In embodiments, the Akt is a mutant Akt. In embodiments, the mutant Akt is associated with a disease that is not associated with wildtype Akt. In embodiments, the Akt may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype Akt. In embodiments, the Akt has the protein sequence corresponding to reference number GI: 62241011.

Methods of Modulating mTOR

In some embodiments, compounds disclosed herein are more selective inhibitors of mTORC1 versus mTORC2. In some embodiments, compounds disclosed herein are more selective inhibitors of mTORC2 versus mTORC1. In some embodiments, compounds disclosed herein exhibit no selectivity difference between mTORC1 and mTORC2.

In another aspect is provided a method of modulating mTORC1 activity in a subject in need thereof, including administering to the subject an effective amount of a compound as described herein, or a pharmaceutically acceptable salt thereof. In embodiments, the method includes inhibiting mTORC1 activity. In embodiments, the method includes inhibiting mTORC1 activity and not inhibiting mTORC2 activity.

In embodiments, the method includes inhibiting mTORC1 activity more than inhibiting mTORC2 activity. In embodiments, the method includes inhibiting mTORC1 activity at least 1.1 fold as much as inhibiting mTORC2 activity (e.g., at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 10000, 20000, 30000, 40000, 50000, 60000, 70000, 80000, 90000, 100000, 100000, 200000, 300000, 400000, 500000, 600000, 700000, 800000, 900000, or 1000000 fold).

In another aspect is provided a method of modulating mTORC2 activity in a subject in need thereof, including administering to the subject an effective amount of a compound as described herein, or a pharmaceutically acceptable salt thereof. In embodiments, the method includes inhibiting mTORC2 activity. In embodiments, the method includes inhibiting mTORC2 activity and not inhibiting mTORC1 activity.

In embodiments, the method includes inhibiting mTORC2 activity more than inhibiting mTORC1 activity. In embodiments, the method includes inhibiting mTORC2 activity at least 1.1 fold as much as inhibiting mTORC1 activity (e.g., at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300.400, 500, 600, 700, 800, 900, 1000, 2000, 3000, 4000, 5000, 6000, 7000, 8000, 9000, 10000, 10000, 20000, 30000, 40000, 50000, 60000, 70000, 80000, 90000, 100000, 100000, 200000, 300000, 400000, 500000, 600000, 700000, 800000, 900000, or 1000000 fold).

In some embodiments, the mTOR is in a cell. In some embodiments, the cell is a mammalian cell, such as a human cell. The cell may be isolated in vitro, form part of a tissue in vitro, or may form part of an organism.

### Exemplary Embodiments

Some embodiments of this disclosure are Embodiment I, as follows:

Embodiment I-1. A compound of Formula I:

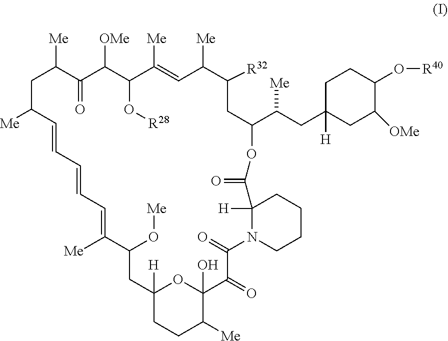

or a pharmaceutically acceptable salt or tautomer thereof, wherein:

R³² is —H, ═O or —OR³;

R²⁸ is —H or —C(═Z¹)—R^{28a};

R⁴⁰ is —H or —C(═Z¹)—R^{40a};
wherein at least one of R²⁸ and R⁴⁰ is not H;

Z¹ is O or S;

R^{28a} and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl is unsubstituted or substituted with 1-5 substituents selected from —NO₂ and halogen;

A¹ and A² are independently absent or are independently selected from

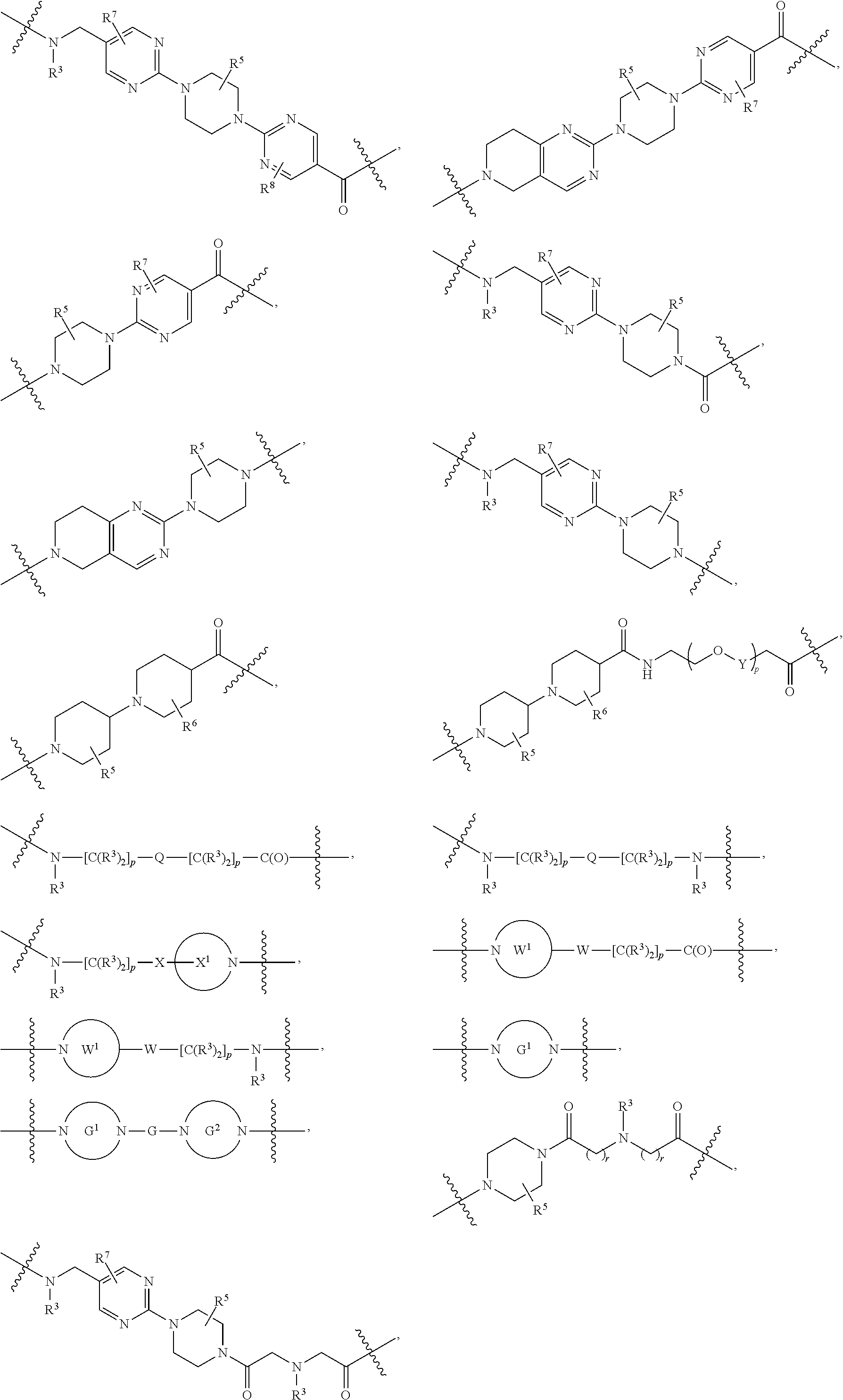

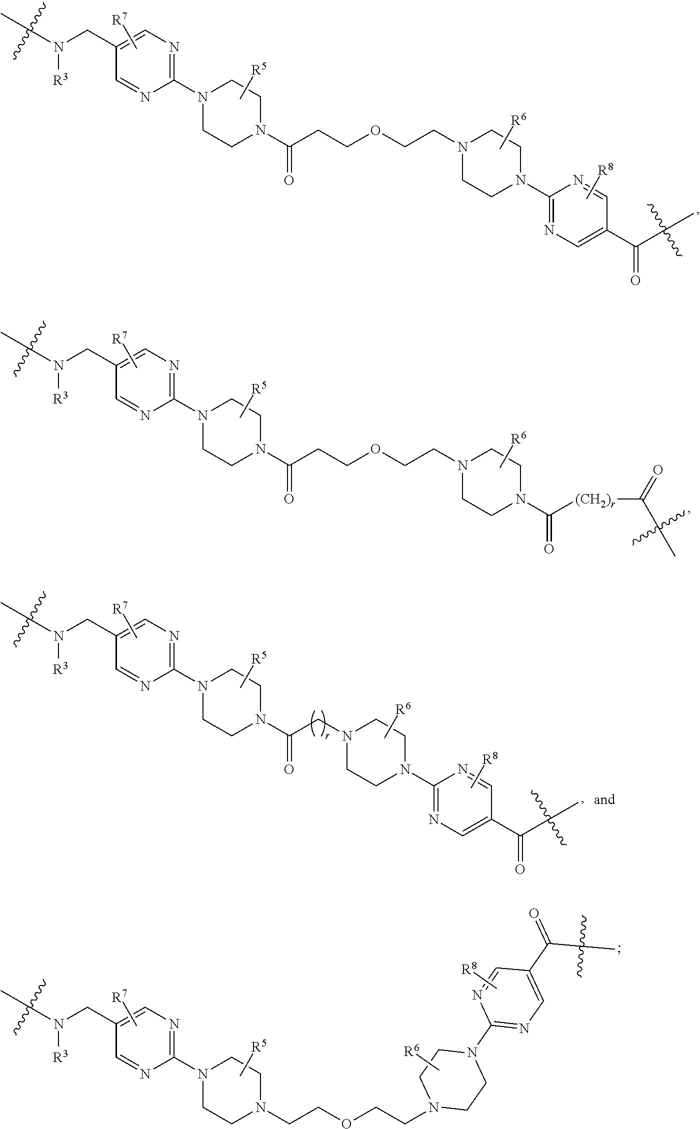

wherein the bond on the left side of A¹, as drawn, is bound to —C(═Z¹)— or L²; and wherein the bond on the right side of the A² moiety, as drawn, is bound to B or L³;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is a heteroarylene or heterocyclylene ring;

each W is independently absent or 1 to 2 rings selected from arylene, cycloalkylene, heteroarylene, and heterocyclylene;

each W¹ is a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

L¹ is selected from

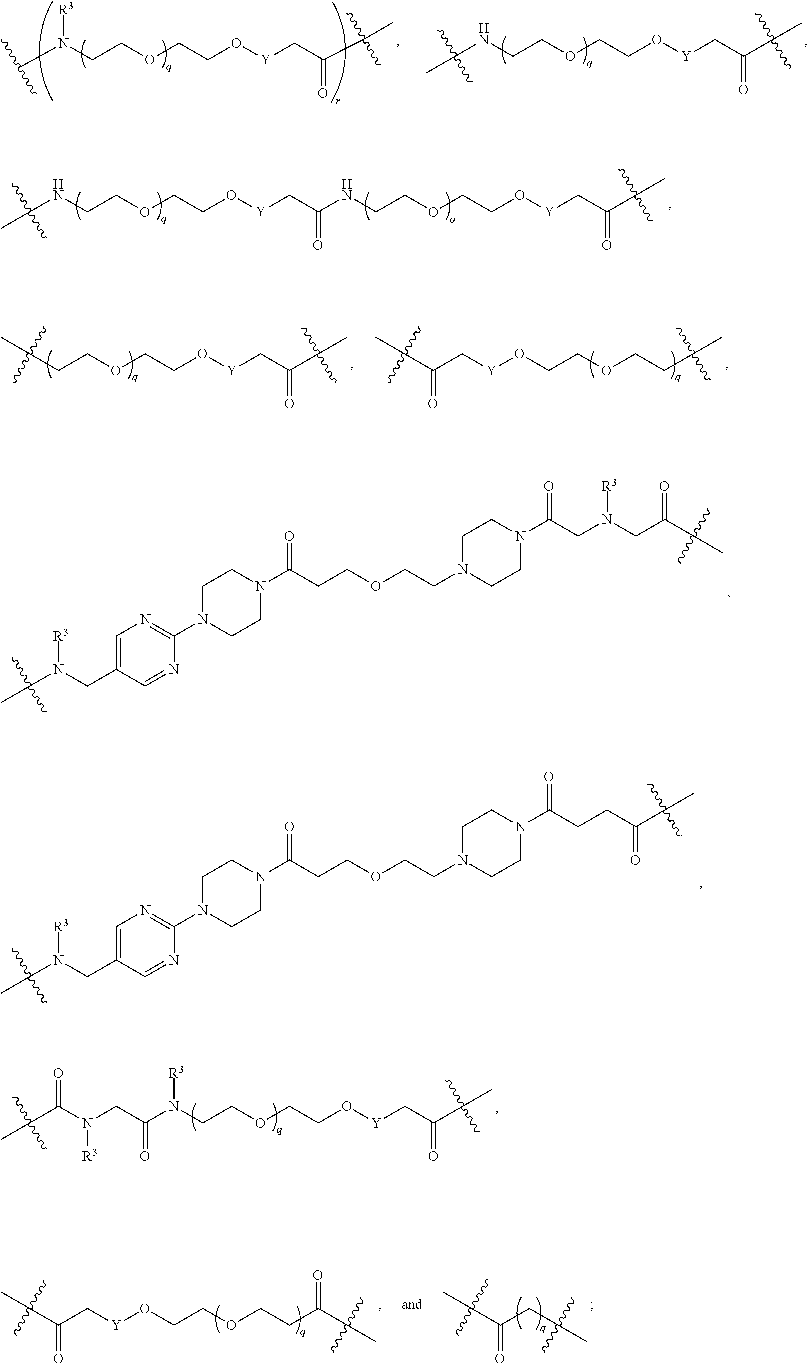

L² and L³ are independently absent or are independently selected from

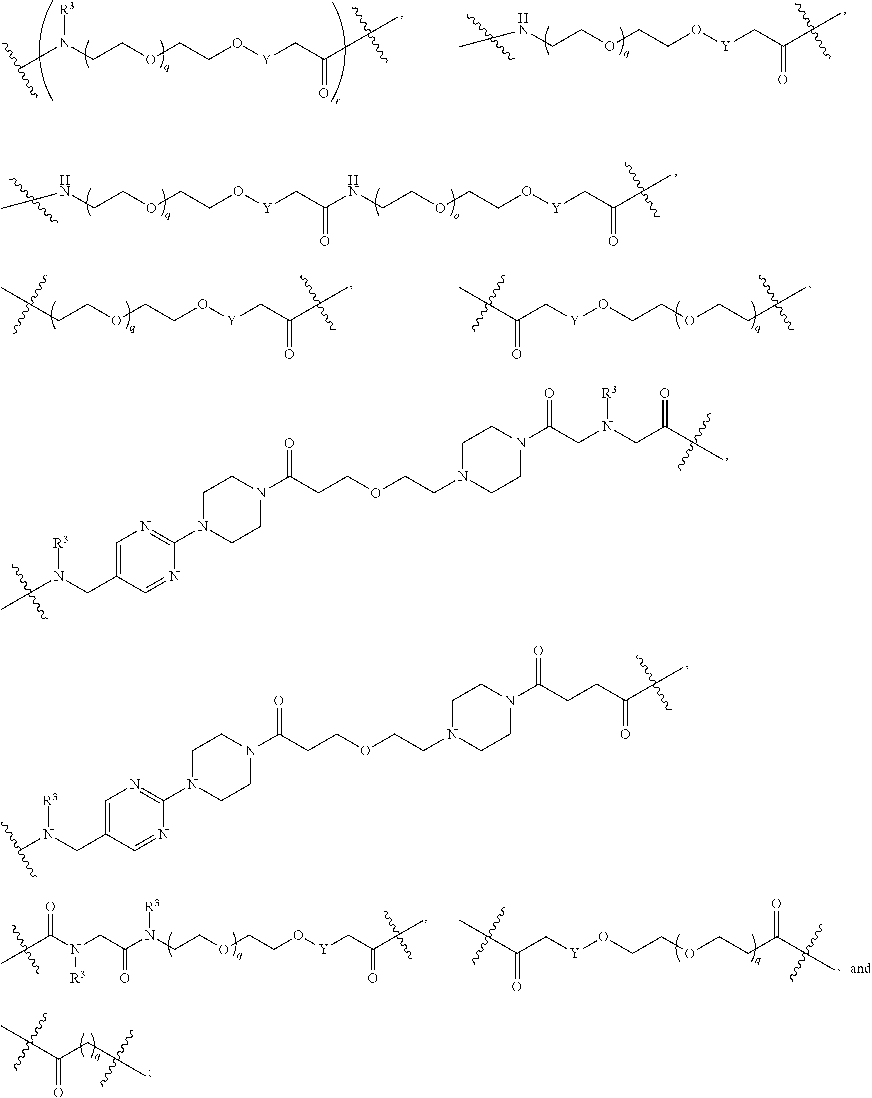

B is selected from

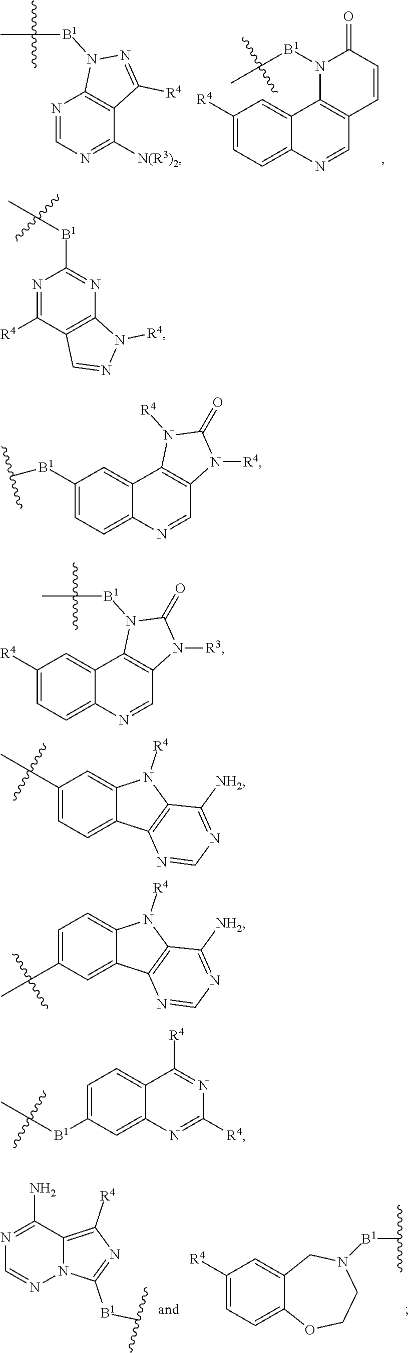

B¹ is selected from

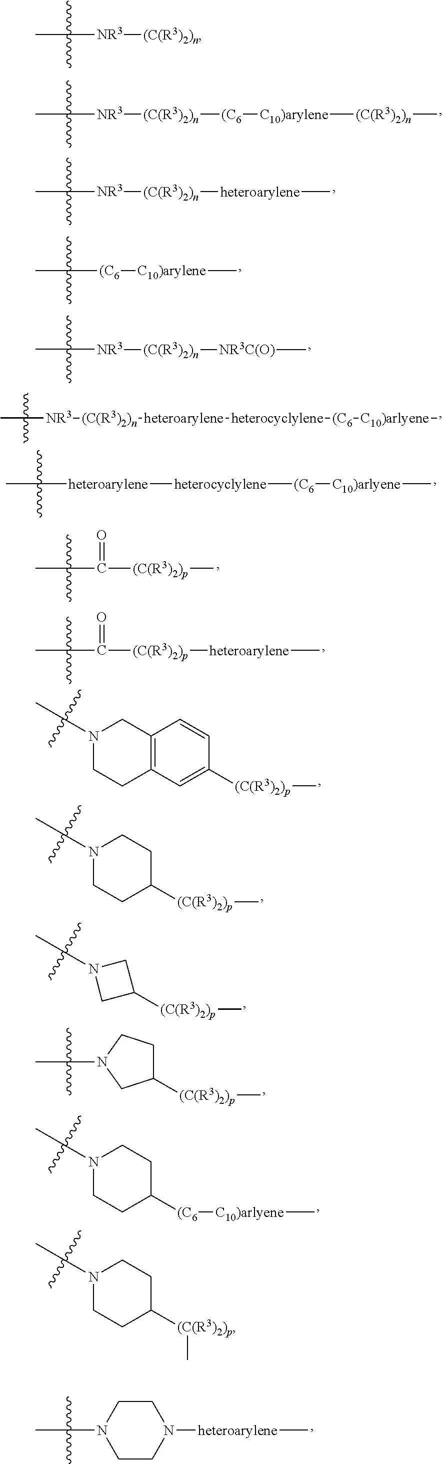

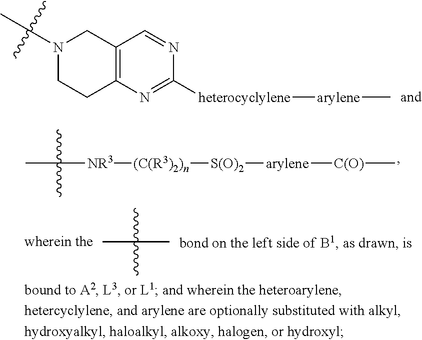

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl; or —C(O)NR³-heterocyclyl;

each R⁵ is independently H, (C₁-C₆)alkyl, —C(O)OR³, or —N(R³)₂, wherein the alkyl is optionally substituted with —N(R³)₂ or —OR³;

each R⁶ is independently H, (C₁-C₆)alkyl, —C(O)OR³, or —N(R³)₂, wherein the alkyl is optionally substituted with —N(R³)₂ or —OR³;

each R⁷ is independently H, (C₁-C₆)alkyl, —C(O)OR³, or —N(R³)₂, wherein the alkyl is optionally substituted with —N(R³)₂ or —OR³;

each R⁸ is independently H, (C₁-C₆)alkyl, —C(O)OR³, or —N(R³)₂, wherein the alkyl is optionally substituted with —N(R³)₂ or —OR³;

each Y is independently C(R³)₂ or a bond;

each n is independently a number from one to 12;

each o is independently a number from zero to 30;

each p is independently a number from zero to 12;

each q is independently a number from zero to 30; and

each r is independently a number from one to 6.

Embodiment I-2. A compound of Formula II:

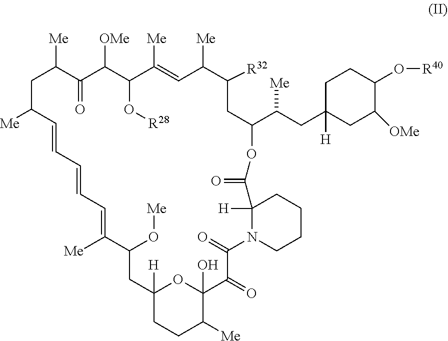

or a pharmaceutically acceptable salt or tautomer thereof, wherein:

R³² is —H, ═O or —OR³;

R²⁸ is —H or —C(═Z¹)—R^{28a};

R⁴⁰ is —H or
wherein at least one of R²⁸ and R⁴⁰ is not H;

Z¹ is O or S;

R^{28a} and R^{40a} are independently -A¹-L¹-A²-B; -A¹-A²-B; —O—(C₁-C₆)alkyl; or —O—(C₆-C₁₀)aryl; wherein the aryl is unsubstituted or substituted with 1-5 substituents selected from —NO₂ and halogen;

A¹ and A² are independently absent or are independently selected from

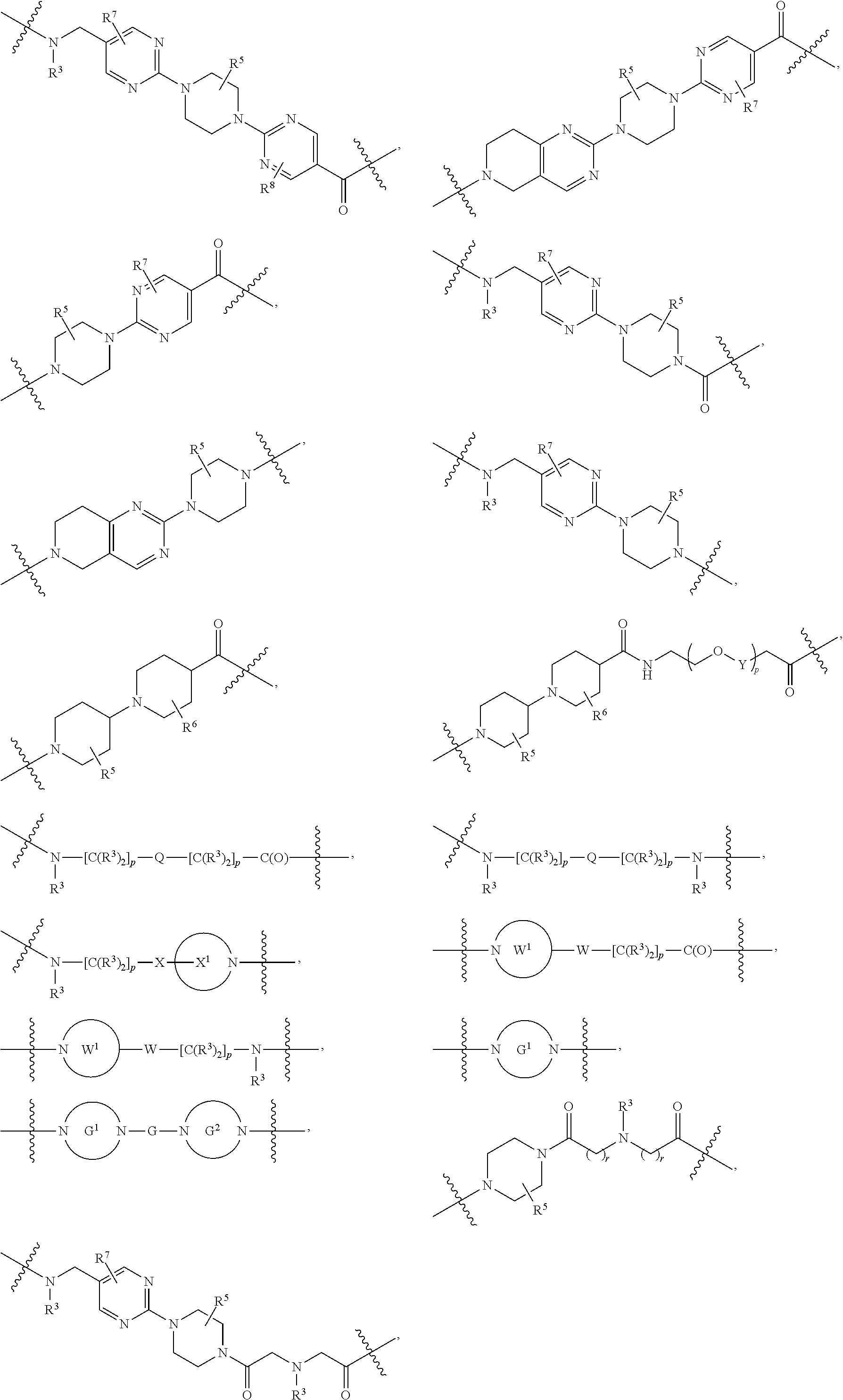

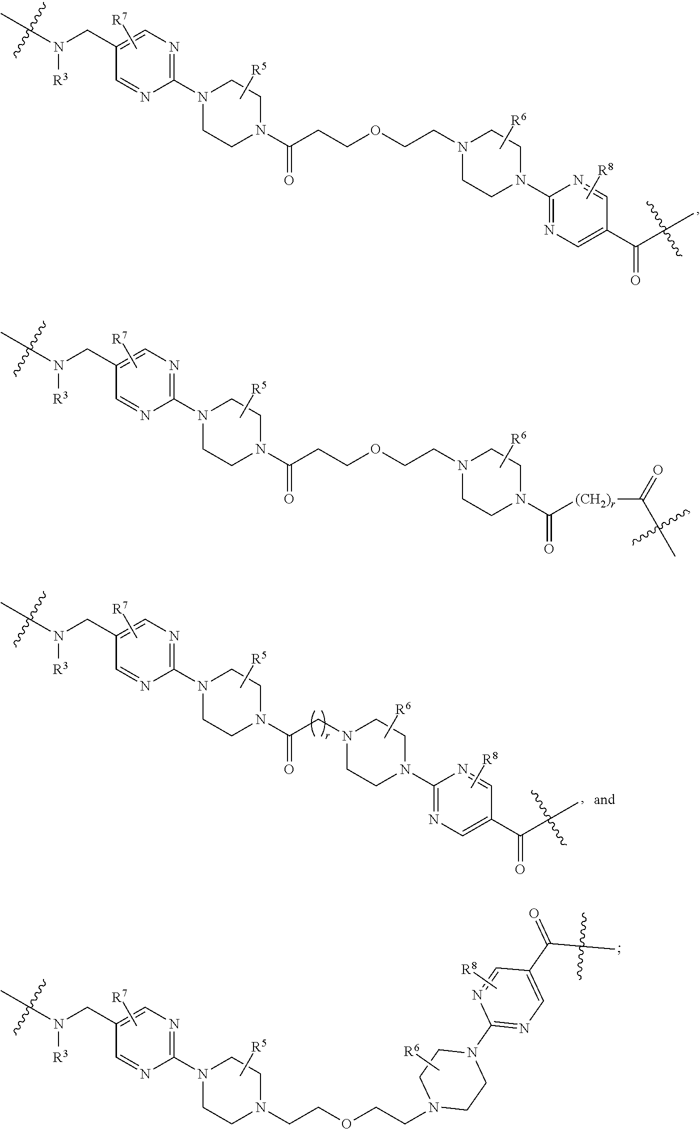

wherein the bond on the left side of A¹, as drawn, is bound to —C(═Z¹)—; and wherein the bond on the right side of the A² moiety, as drawn, is bound to B;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is a heteroarylene or heterocyclylene ring;

each W is independently absent or 1 to 2 rings selected from arylene, cycloalkylene, heteroarylene, and heterocyclylene;

each W¹ is a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

L¹ is selected from

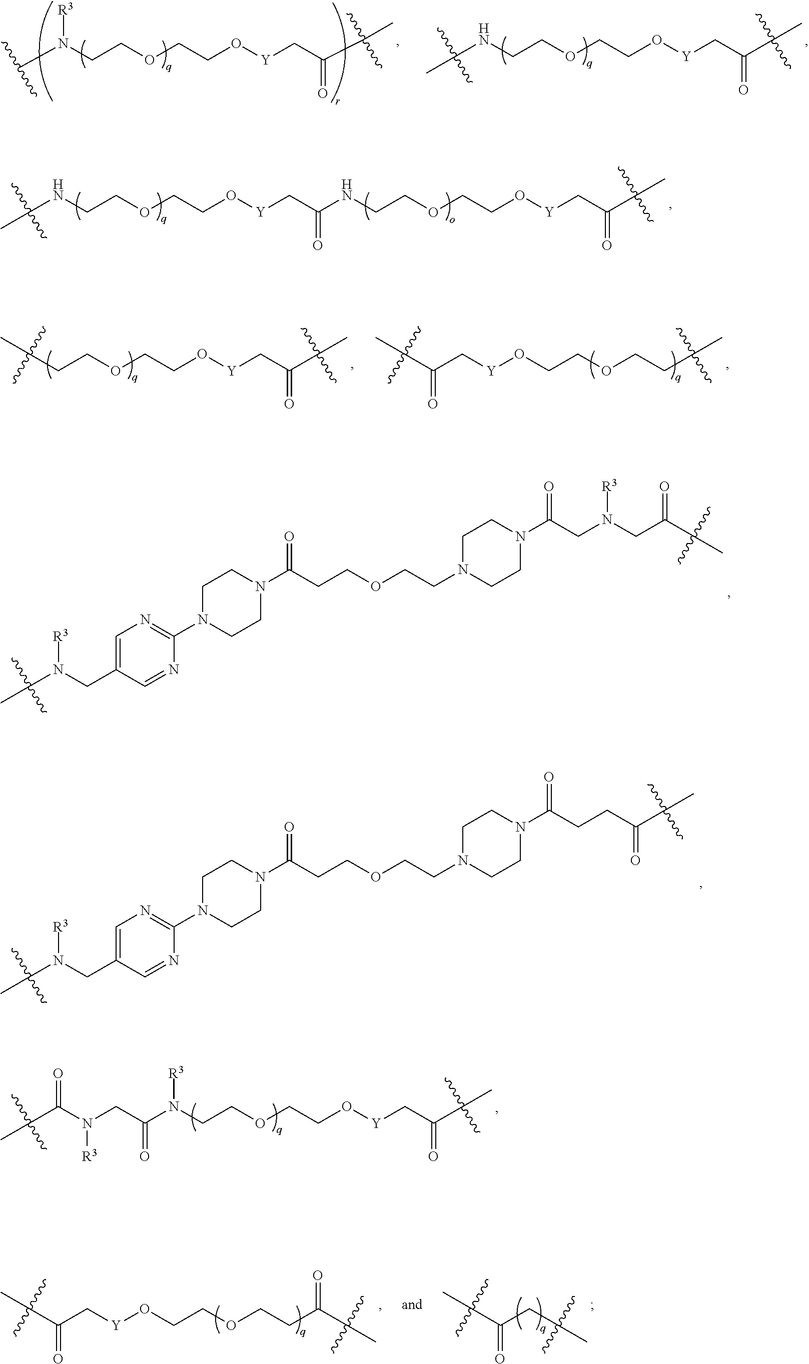

B is selected from

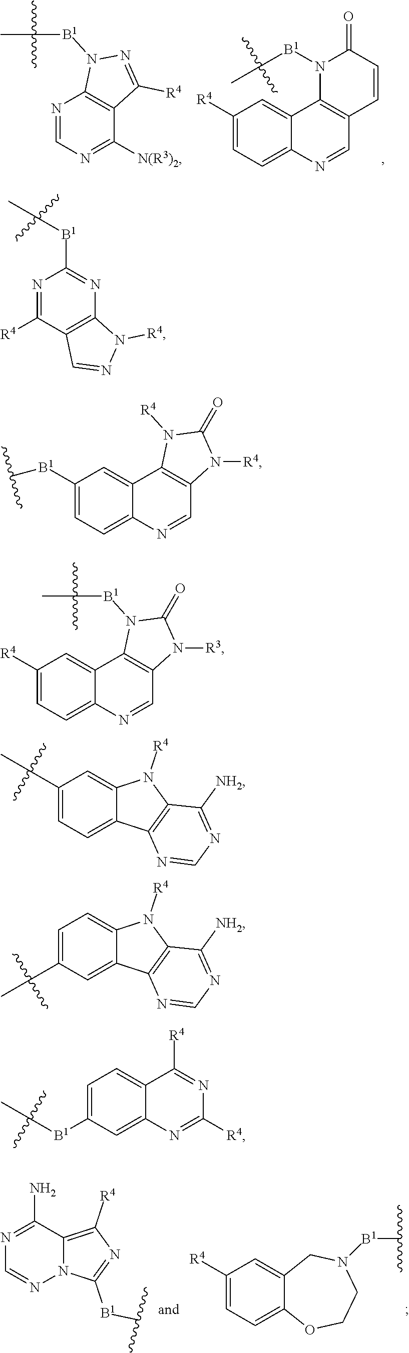

B¹ is selected from

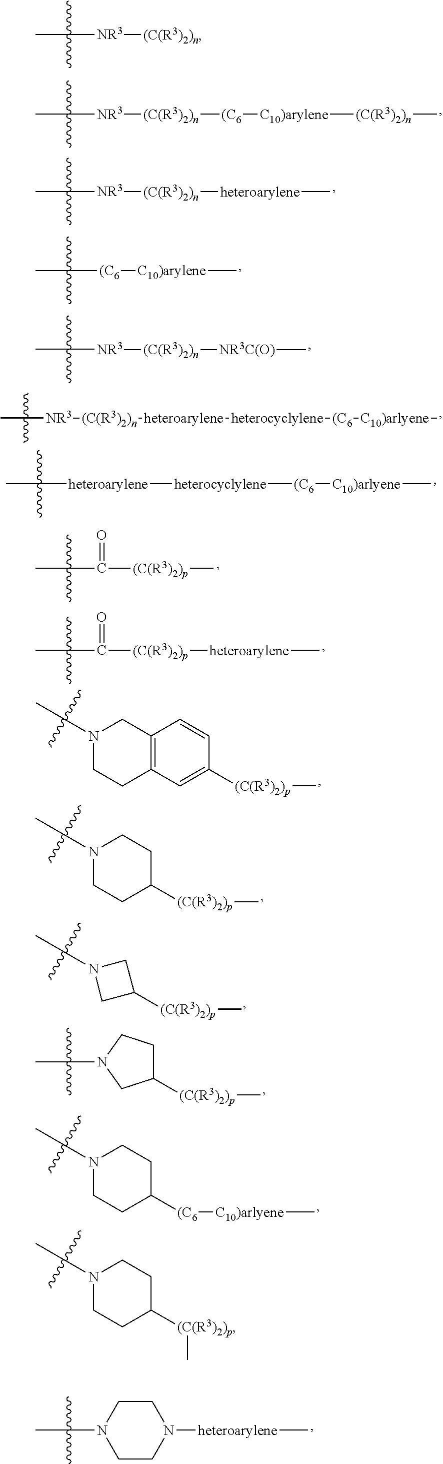

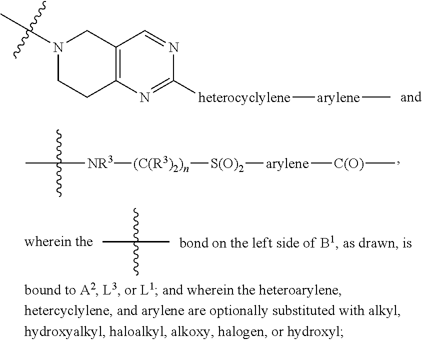

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl; or —C(O)NR³-heterocyclyl;

each R⁵ is independently H, (C₁-C₆)alkyl, —C(O)OR³, or —N(R³)₂, wherein the alkyl is optionally substituted with —N(R³)₂ or —OR³;

each R⁶ is independently H, (C₁-C₆)alkyl, —C(O)OR³, or —N(R³)₂, wherein the alkyl is optionally substituted with —N(R³)₂ or —OR³;

each R⁷ is independently H, (C₁-C₆)alkyl, —C(O)OR³, or —N(R³)₂, wherein the alkyl is optionally substituted with —N(R³)₂ or —OR³;

each R⁸ is independently H, (C₁-C₆)alkyl, —C(O)OR³, or —N(R³)₂, wherein the alkyl is optionally substituted with —N(R³)₂ or —OR³;

each Y is independently C(R³)₂ or a bond;

each n is independently a number from one to 12;

each o is independently a number from zero to 30;

each p is independently a number from zero to 12;

each q is independently a number from zero to 30; and

each r is independently a number from one to 6.

Embodiment I-3. The compound of Embodiment I-1 or I-2, wherein R³² is ═O.

Embodiment I-4. The compound of Embodiment I-1 or I-2, wherein R³² is —OR³.

Embodiment I-5. The compound of any one of Embodiments I-1 to I-4, wherein the compounds are represented by the structure of Formula I-40:

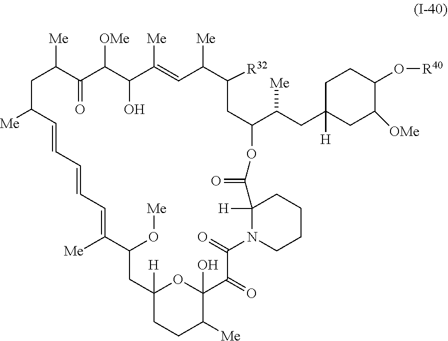

or a pharmaceutically acceptable salt or tautomer thereof.

Embodiment I-6. The compound of Embodiment I-5, wherein Z¹ is O.

Embodiment I-7. The compound of Embodiment I-5, wherein Z¹ is S.

Embodiment I-8. The compound of any one of Embodiments I-5 to I-7, wherein R^{40a} is -A¹-L¹-A²-B, wherein A¹ and A² are absent.

Embodiment I-9. The compound of any one of Embodiments I-5 to I-7, wherein R^{40a} is -A¹-L¹-A²-B, wherein A² is absent.

Embodiment I-10. The compound of any one of Embodiments I-5 to I-7, wherein R^{40a} is -A¹-L¹-A²-B, wherein A¹ is absent.

Embodiment I-11. The compound of any one of Embodiments I-5 to I-7, wherein R^{40a} is -A¹-L¹-A²-B.

Embodiment I-12. The compound of any one of Embodiments I-5 to I-7, wherein R^{40a} is -A¹-A²-B.

Embodiment I-13. The compound of any one of Embodiments I-5 to I-7, wherein R^{40a} is -L²-A¹-L¹-A²-L³-B, wherein L² and A¹ are absent.

Embodiment I-14. The compound of any one of Embodiments I-5 to I-7, wherein R^{40a} is -L²-A¹-L¹-A²-L³-B, wherein L² is absent.

Embodiment I-15. The compound of any one of Embodiments I-5 to I-7, wherein R^{40a} is -L²-A¹-L¹-A²-L³-B, wherein L³ is absent.

Embodiment I-16. The compound of any one of Embodiments I-5 to I-7, wherein R^{40a} is —O—(C₁-C₆)alkyl or —O—(C₆-C₁₀)aryl; wherein the aryl is unsubstituted or substituted with 1-5 substituents selected from —NO₂ and halogen.

Embodiment I-17. The compound of any one of Embodiments I-1 to I-4, wherein the compounds are represented by the structure of Formula I-28:

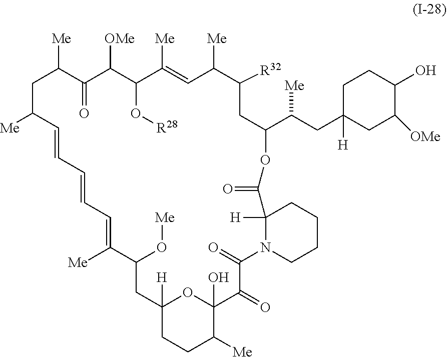

or a pharmaceutically acceptable salt or tautomer thereof.

Embodiment I-18. The compound of Embodiment I-17, wherein Z¹ is O.

Embodiment I-19. The compound of Embodiment I-17, wherein Z¹ is S.

Embodiment I-20. The compound of any one of Embodiments I-17 to I-19, wherein R^{28a} is -A¹-L¹-A²-B, wherein A¹ and A² are absent.

Embodiment I-21. The compound of any one of Embodiments I-17 to I-19, wherein R^{28a} is -A¹-L¹-A²-B, wherein A² is absent.

Embodiment I-22. The compound of any one of Embodiments I-17 to I-19, wherein R^{28a} is -A¹-L¹-A²-B, wherein A¹ is absent.

Embodiment I-23. The compound of any one of Embodiments I-17 to I-19, wherein R^{28a} is -A¹-L¹-A²-B.

Embodiment I-24. The compound of any one of Embodiments I-17 to I-19, wherein R^{28a} is -A¹-A²-B.

Embodiment I-25. The compound of any one of Embodiments I-17 to I-19, wherein R^{28a} is -L²-A¹-L¹-A²-L³-B, wherein L² and A¹ are absent.

Embodiment I-26. The compound of any one of Embodiments I-17 to I-19, wherein R^{28a} is -L²-A¹-L¹-A²-L³-B, wherein L² is absent.

Embodiment I-27. The compound of any one of Embodiments I-17 to I-19, wherein R^{28a} is -L²-A¹-L¹-A²-L³-B, wherein L³ is absent.

Embodiment I-28. The compound of any one of Embodiments I-17 to I-19, wherein R^{28a} is —O—(C₁-C₆)alkyl or —O—(C₆-C₁₀)aryl; wherein the aryl is unsubstituted or substituted with 1-5 substituents selected from —NO₂ and halogen.

Embodiment I-29. The compound of any one of Embodiments I-1 to I-11, I-13 to I-15, I-17 to I-23, and I-25 to I-27, wherein L¹ is

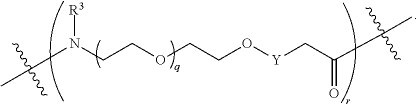

Embodiment I-30. The compound of any one of Embodiments I-1 to I-11, I-13 to I-15, I-17 to I-23, and I-25 to I-27, wherein L¹ is

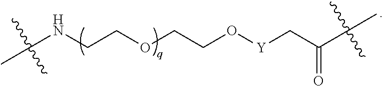

Embodiment I-31. The compound of any one of Embodiments I-1 to I-11, I-13 to I-15 I-17 to I-23 and I-25 to I-27 wherein L¹ is

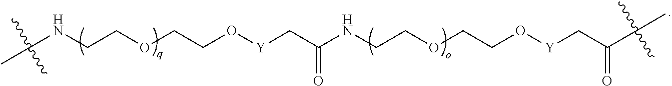

Embodiment I-32. The compound of any one of Embodiments I-1 to I-11, I-13 to I-15, I-17 to I-23, and I-25 to I-27, wherein L¹ is

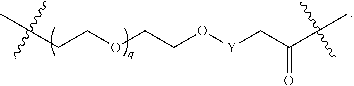

Embodiment I-33. The compound of an one of Embodiments I-1 to I-11, I-13 to I-15, I-17 to I-23, and I-25 to I-27, wherein L¹ is

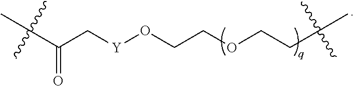

Embodiment I-34. The compound of any one of Embodiments I-1 to I-7, I-15, I-17 to I-19, and I-27, wherein L² is

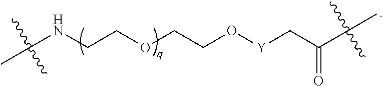

Embodiment I-35. The compound of any one of Embodiments I-1 to I-7, I-13 to I-14, I-17 to I-19, and I-25 to I-26, wherein L³ is

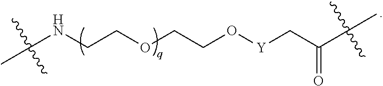

Embodiment I-36. The compound of any one of Embodiments I-1 to I-8, I-10, I-13, I-17 to I-19, I-20, I-22, I-25 and I-29 to I-35, wherein A¹ is absent.

Embodiment I-37. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

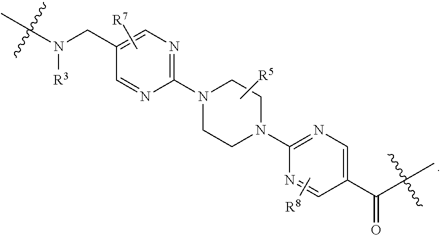

Embodiment I-38. The compound of any one of Embodiments I-1 to I-7, I-9, I-11 to 1-12, I-14 to I-15, I-17 to I-19, I-21, I-23 to I-24, I-26 to I-27, and I-29 to I-35, wherein A¹ is

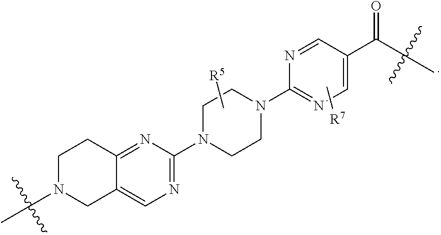

Embodiment I-39. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

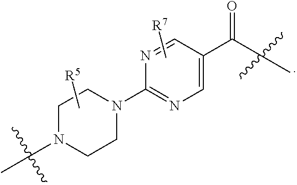

Embodiment I-40. The compound of any one of Embodiments I-1 to 1-7, I-9, I-11 to I-12, I-14 to I-15, I-17 to I-19, I-21, I-23 to I-24, I-26 to I-27, and I-29 to I-35, wherein A¹ is

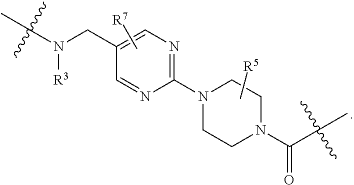

Embodiment I-41. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

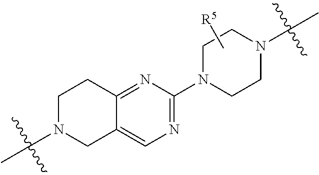

Embodiment I-42. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

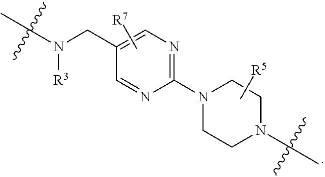

Embodiment I-43. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

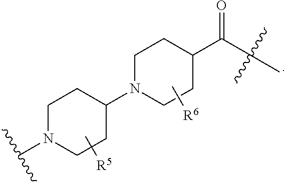

Embodiment I-44. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

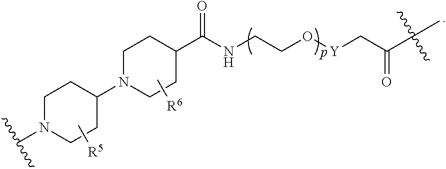

Embodiment I-45. The compound of any one of Embodiments I-1 to 1-9, I-17 to I-21, and I-29 to I-44, wherein A² is absent.

Embodiment I-46. The compound of any one of Embodiments I-1 to I-7, I-10 to I-15, I-17 to I-19, I-22 to I-27 and I-29 to I-44, wherein A² is

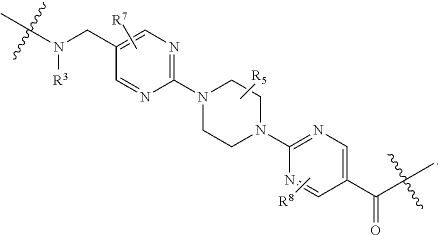

Embodiment I-47. The compound of any one of Embodiments I-1 to I-7, I-10 to I-15, I-17 to I-19, I-22 to I-27 and I-29 to I-44, wherein A² is

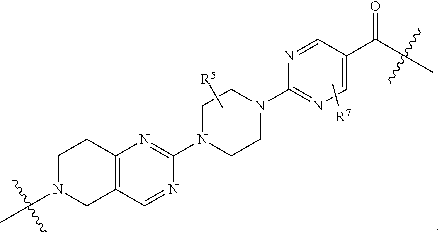

Embodiment I-48. The compound of any one of Embodiments I-1 to I-7, I-10 to I-15, I-17 to I-19, I-22 to I-27 and I-29 to I-44, wherein A² is

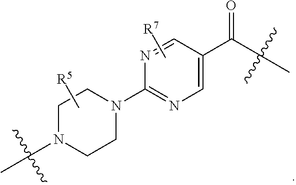

Embodiment I-49. The compound of any one of Embodiments I-1 to I-7, I-10 to I-15, I-17 to I-19, I-22 to I-27 and I-29 to I-44, wherein A² is

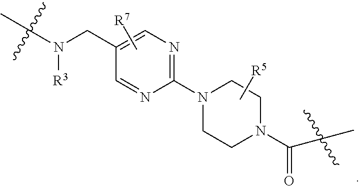

Embodiment I-50. The compound of any one of Embodiments I-1 to I-7, I-10 to I-15, I-17 to I-19, I-22 to I-27 and I-29 to I-44, wherein A² is

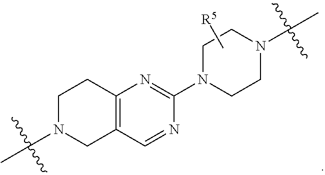

Embodiment I-51. The compound of any one of Embodiments I-1 to I-7, I-10 to I-15, I-17 to I-19, I-22 to I-27 and I-29 to I-44, wherein A² is

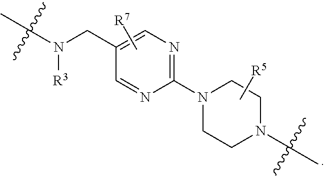

Embodiment I-52. The compound of any one of Embodiments I-1 to I-7, I-10 to I-15, I-17 to I-19, I-22 to I-27 and I-29 to I-44, wherein A² is

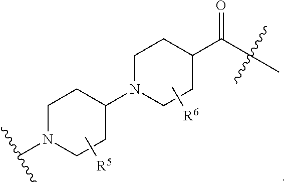

Embodiment I-53. The compound of any one of Embodiments I-1 to I-7, I-10 to I-15, I-17 to I-19, I-22 to I-27 and I-29 to I-44, wherein A² is

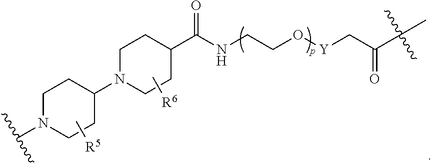

Embodiment I-54. The compound of any one of Embodiments I-1 to I-53, wherein B is

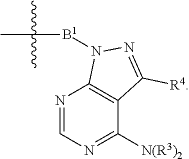

Embodiment I-55. The compound of any one of Embodiments I-1 to I-53, wherein B is

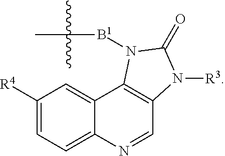

Embodiment I-56. The compound of any one of Embodiments I-1 to I-53, wherein B¹ is

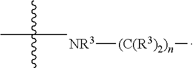

Embodiment I-57. The compound of any one of Embodiments I-1 to I-53, wherein B¹ is

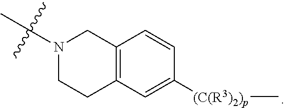

Embodiment I-58. The compound of any one of Embodiments I-1 to I-57, wherein R⁴ is 5-12 membered heteroaryl, optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl.

Embodiment I-59. The compound of any one of Embodiments I-1 to I-58, or a pharmaceutically acceptable salt or tautomer thereof, wherein compound has the following formula:

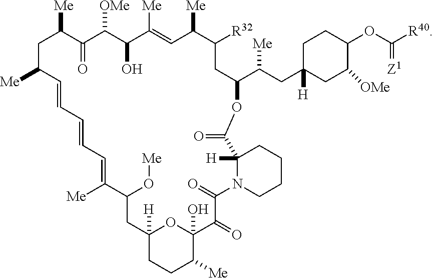

Embodiment I-60. A compound selected from the group consisting of:

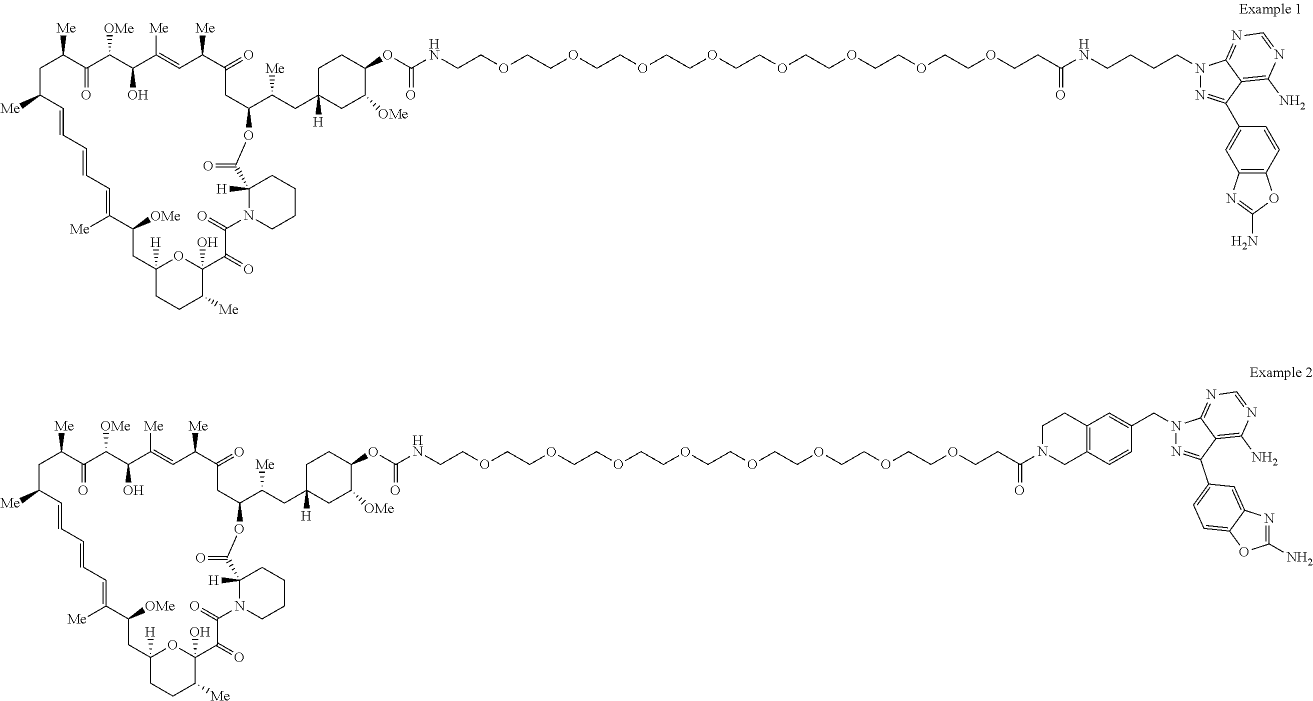

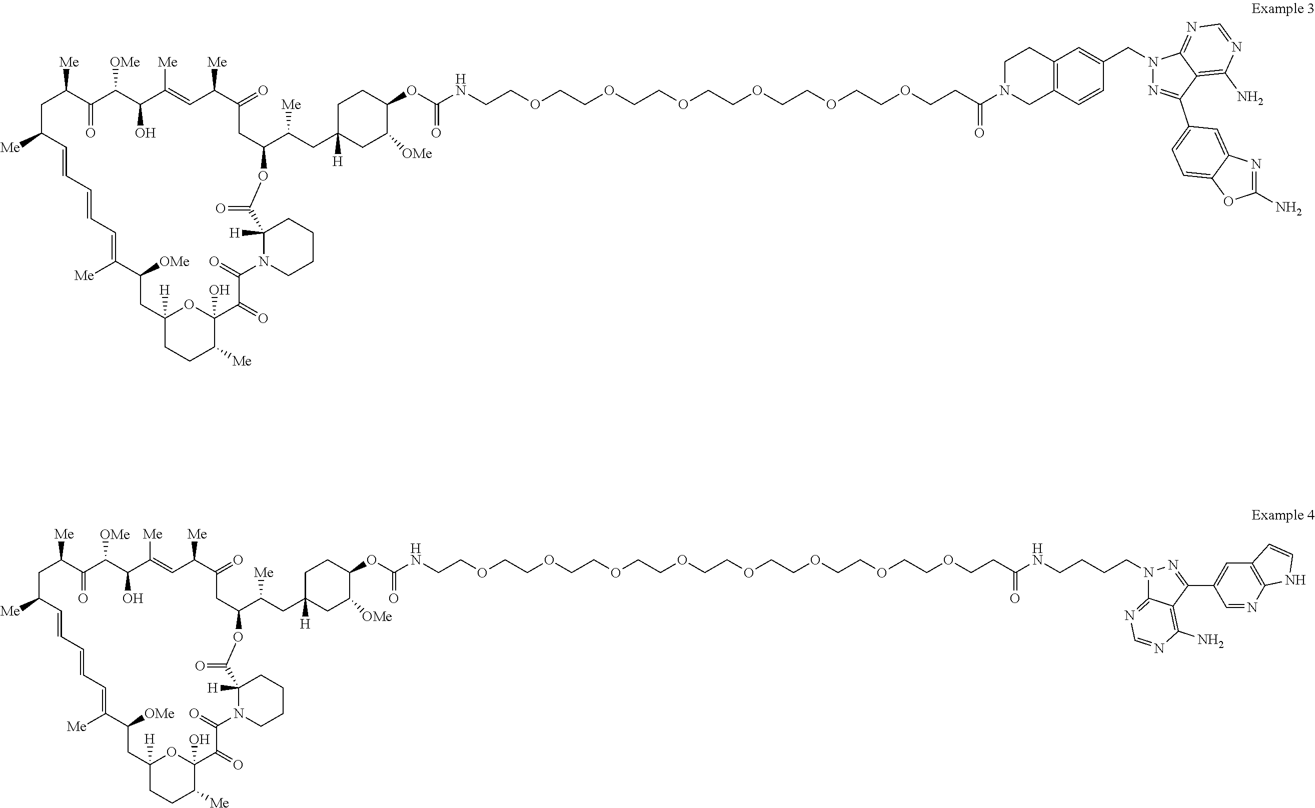

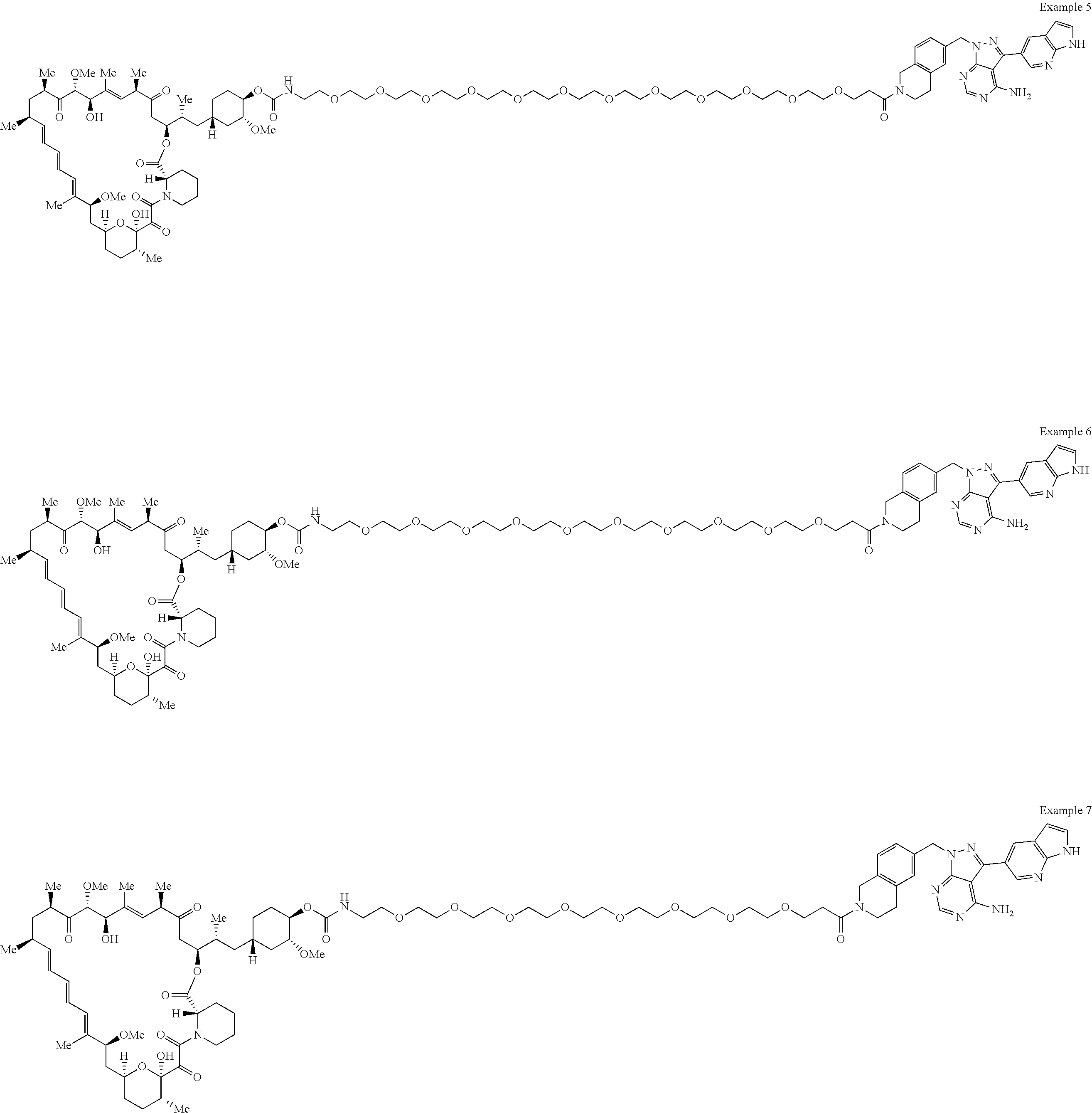

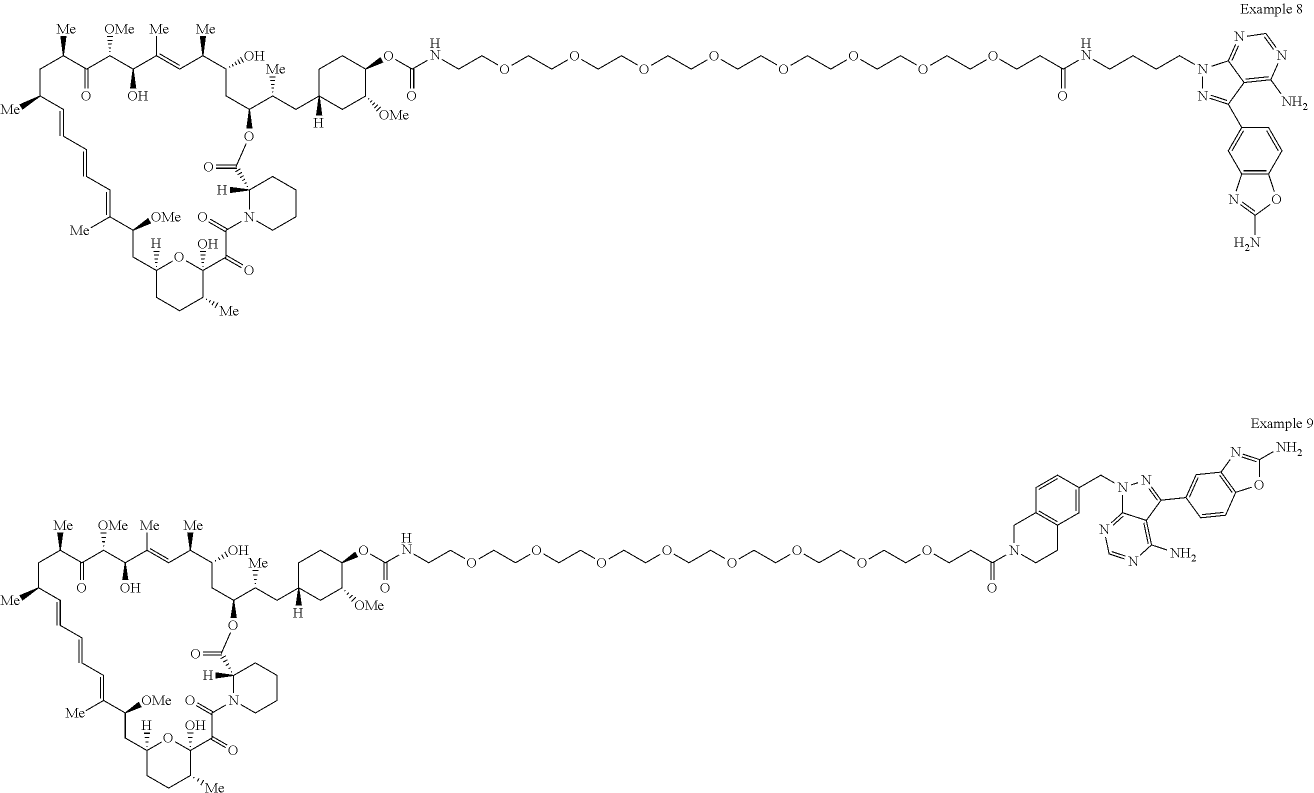

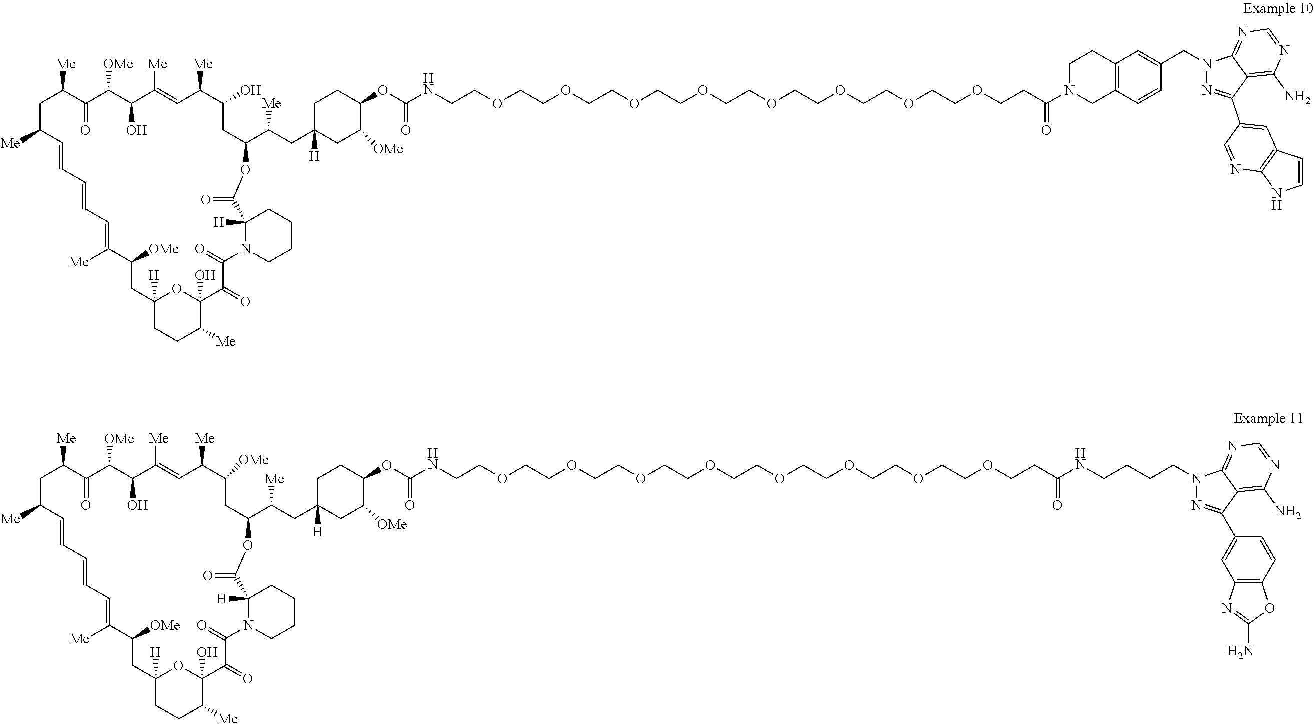

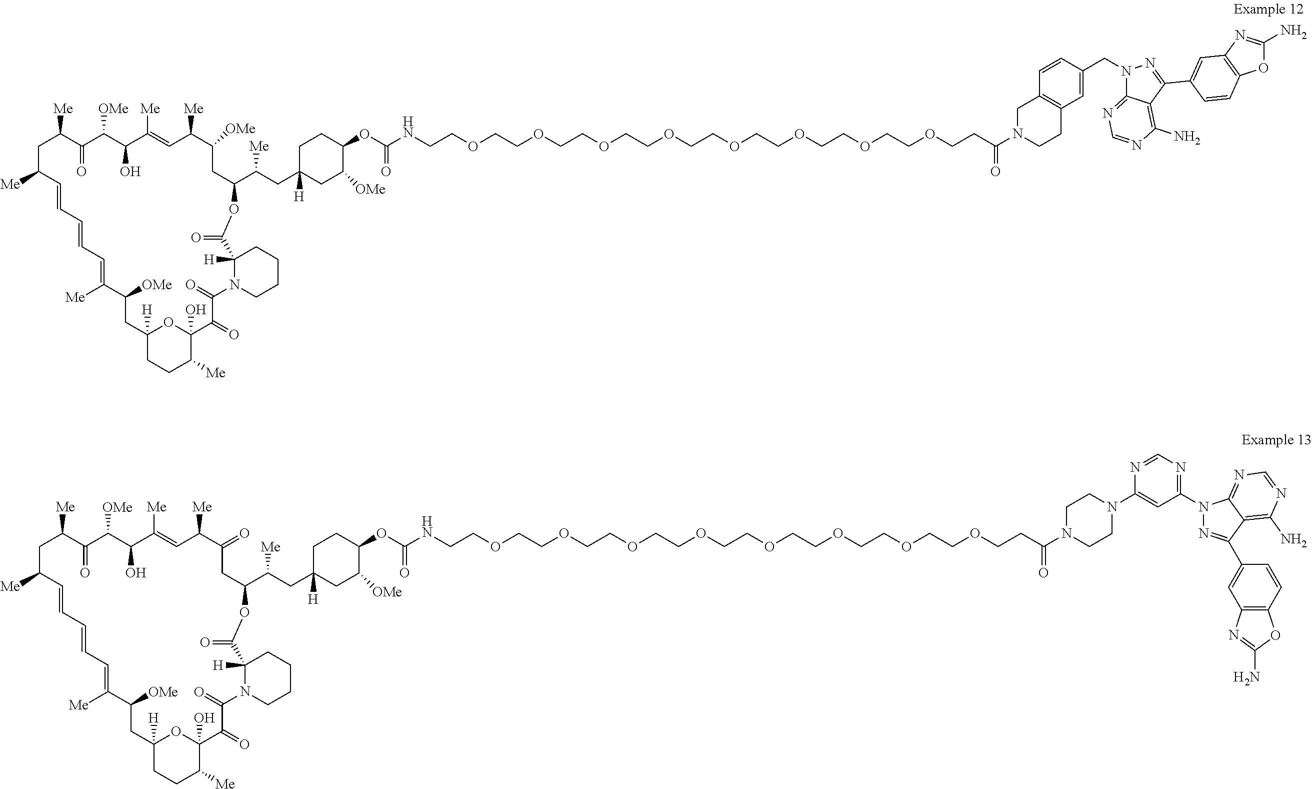

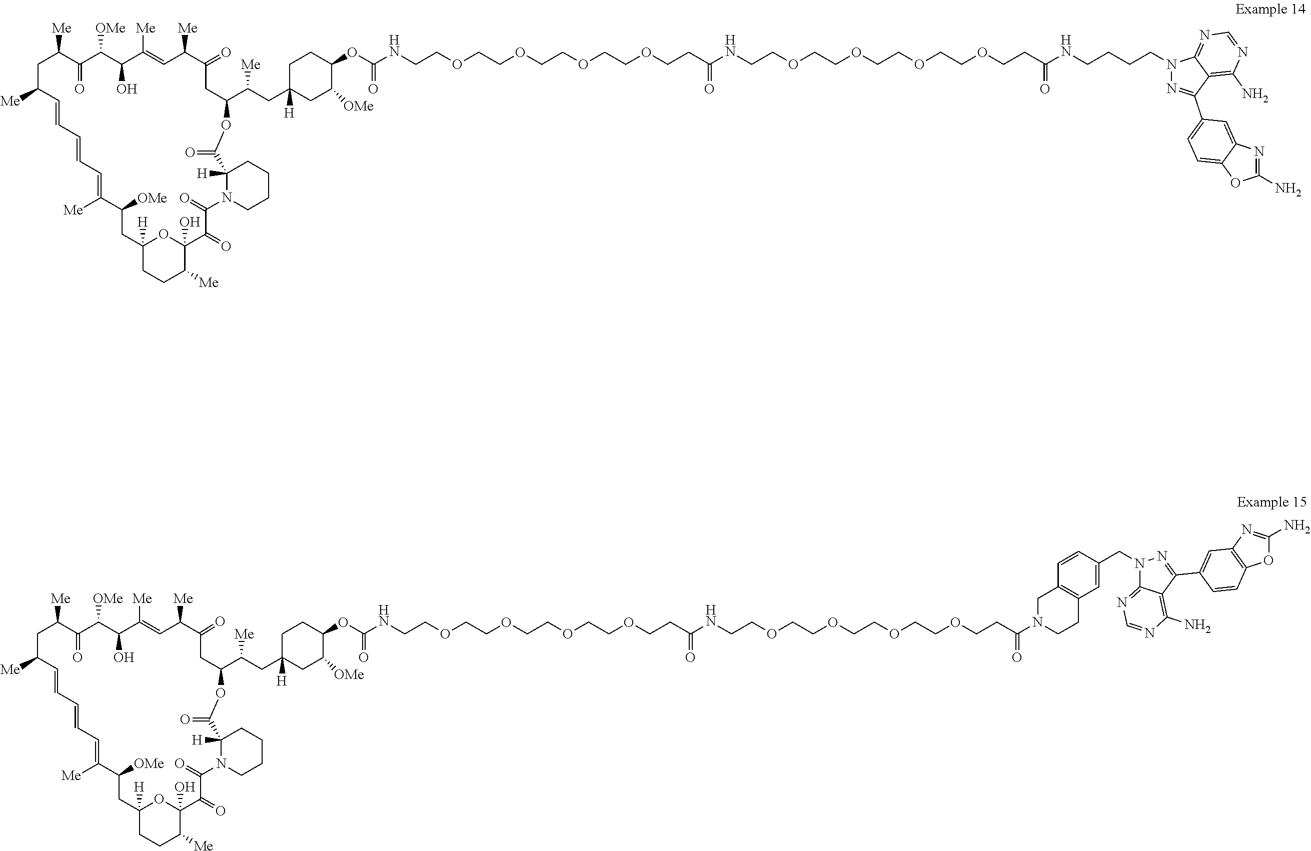

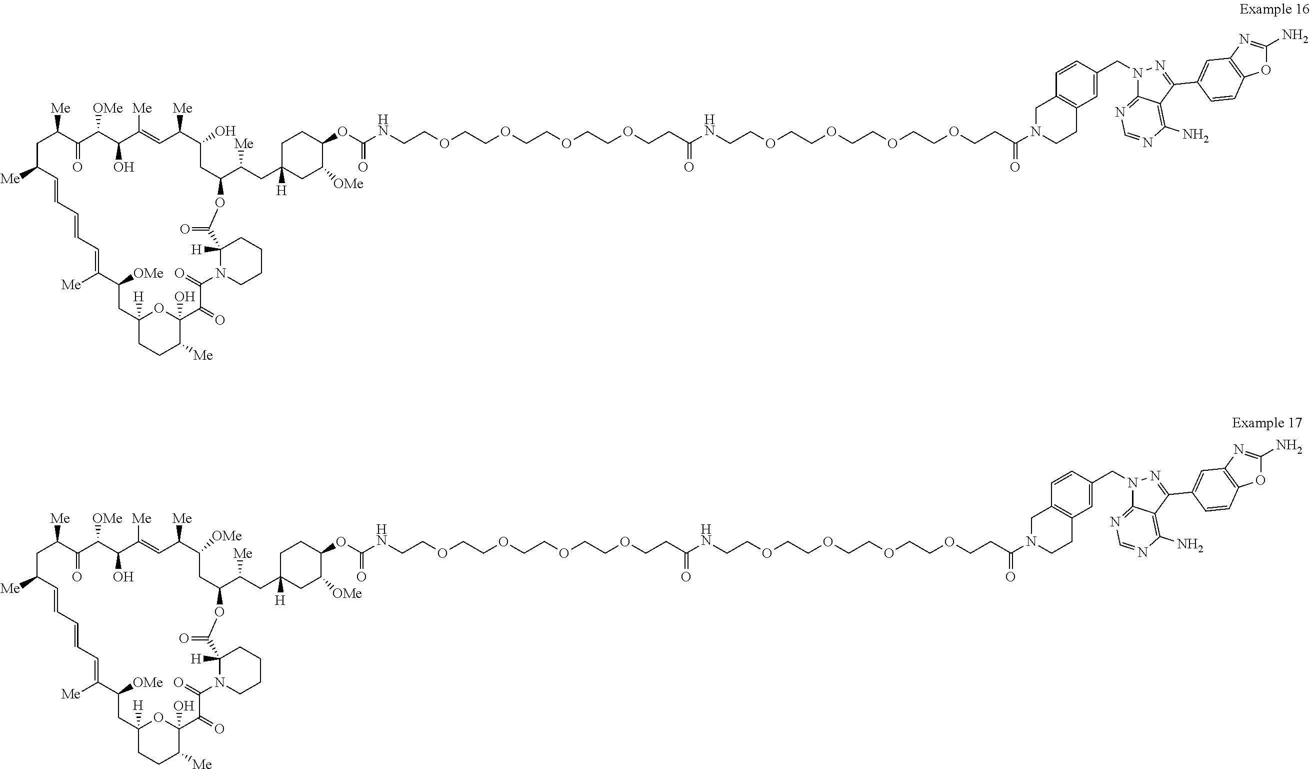

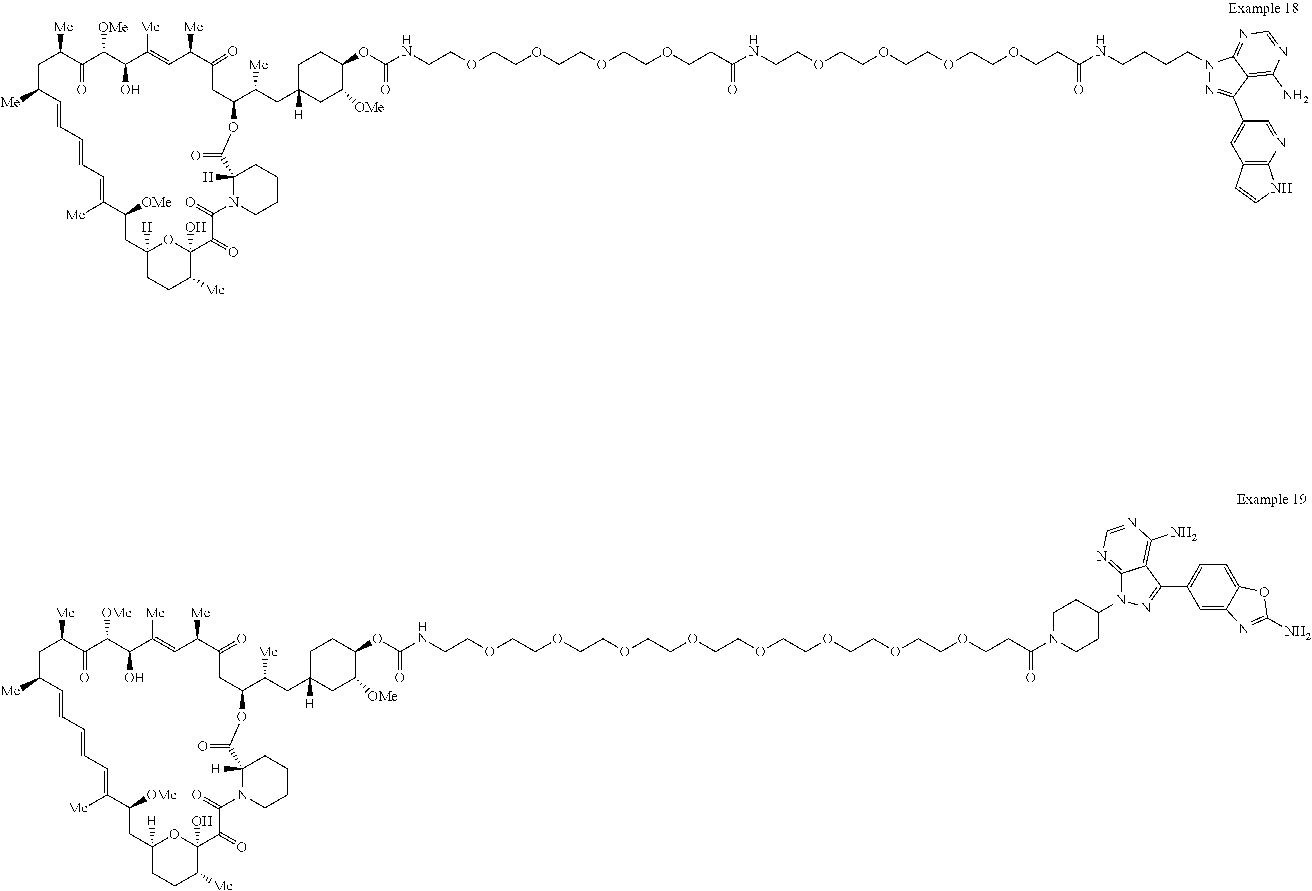

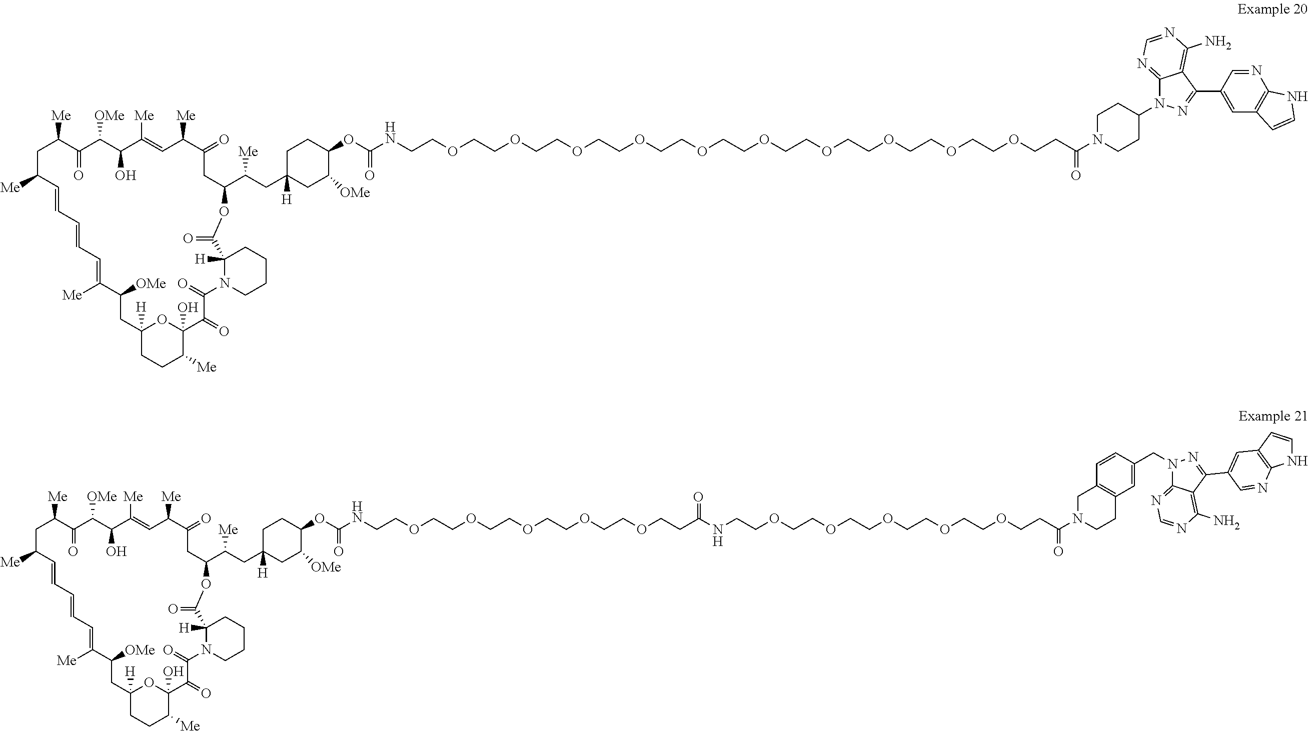

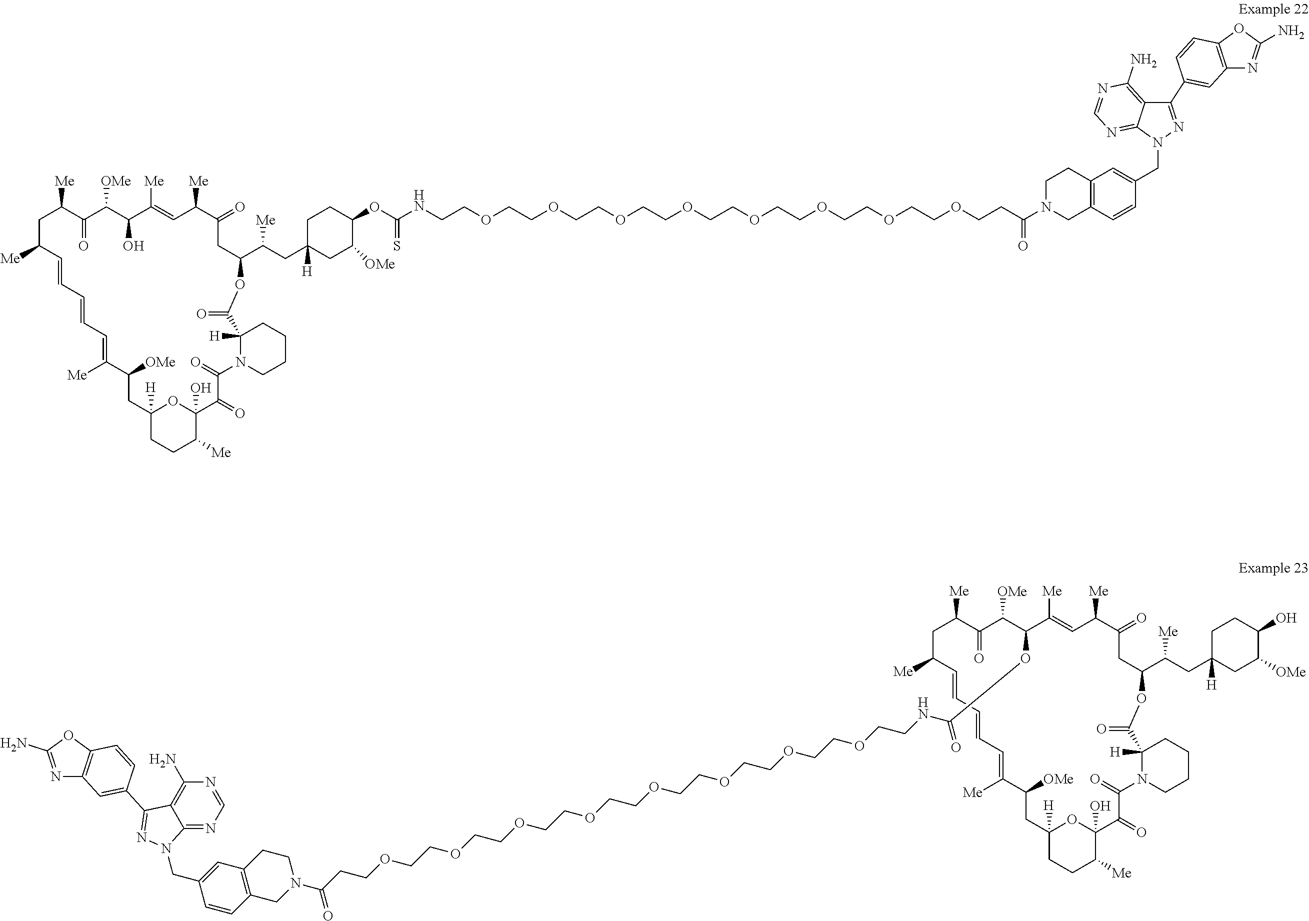

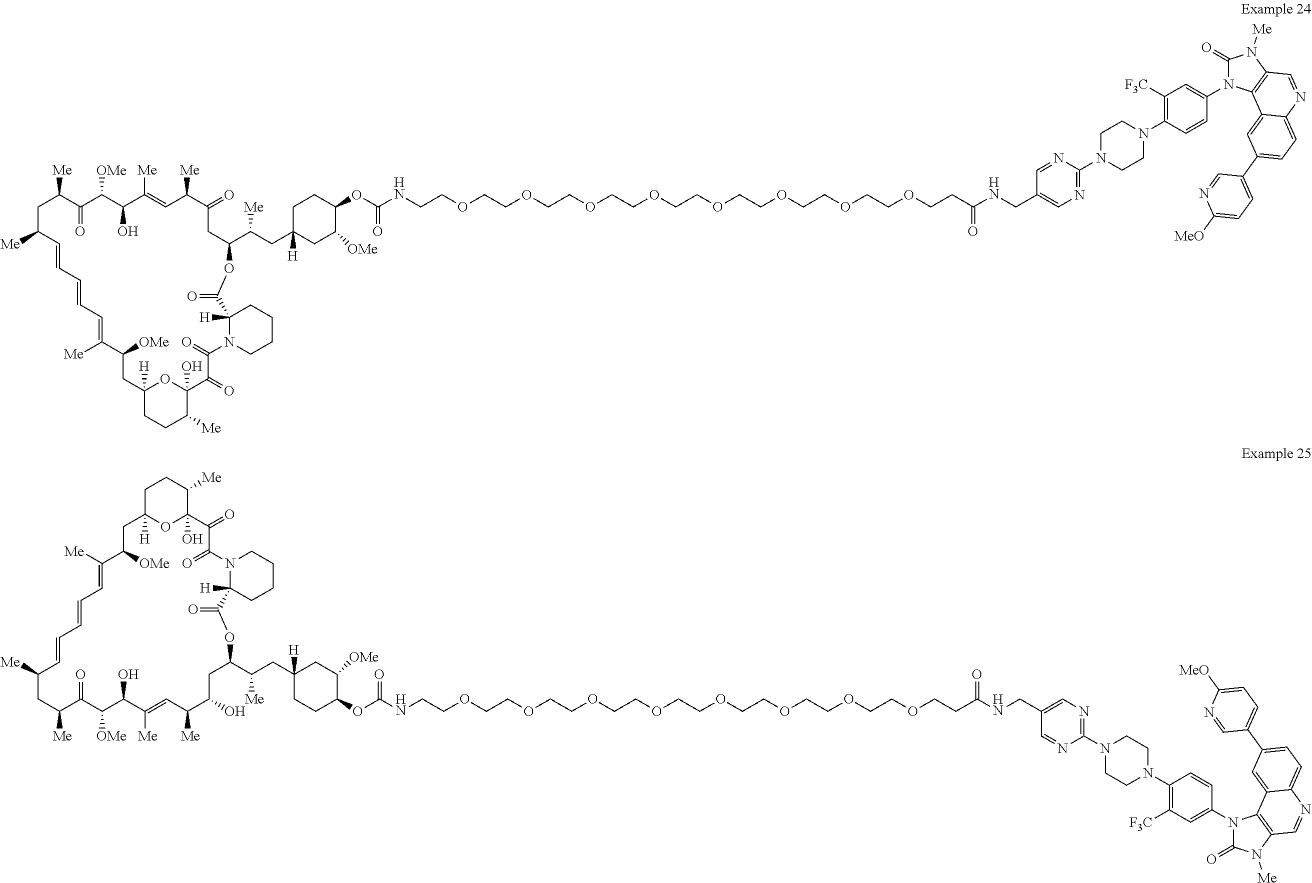

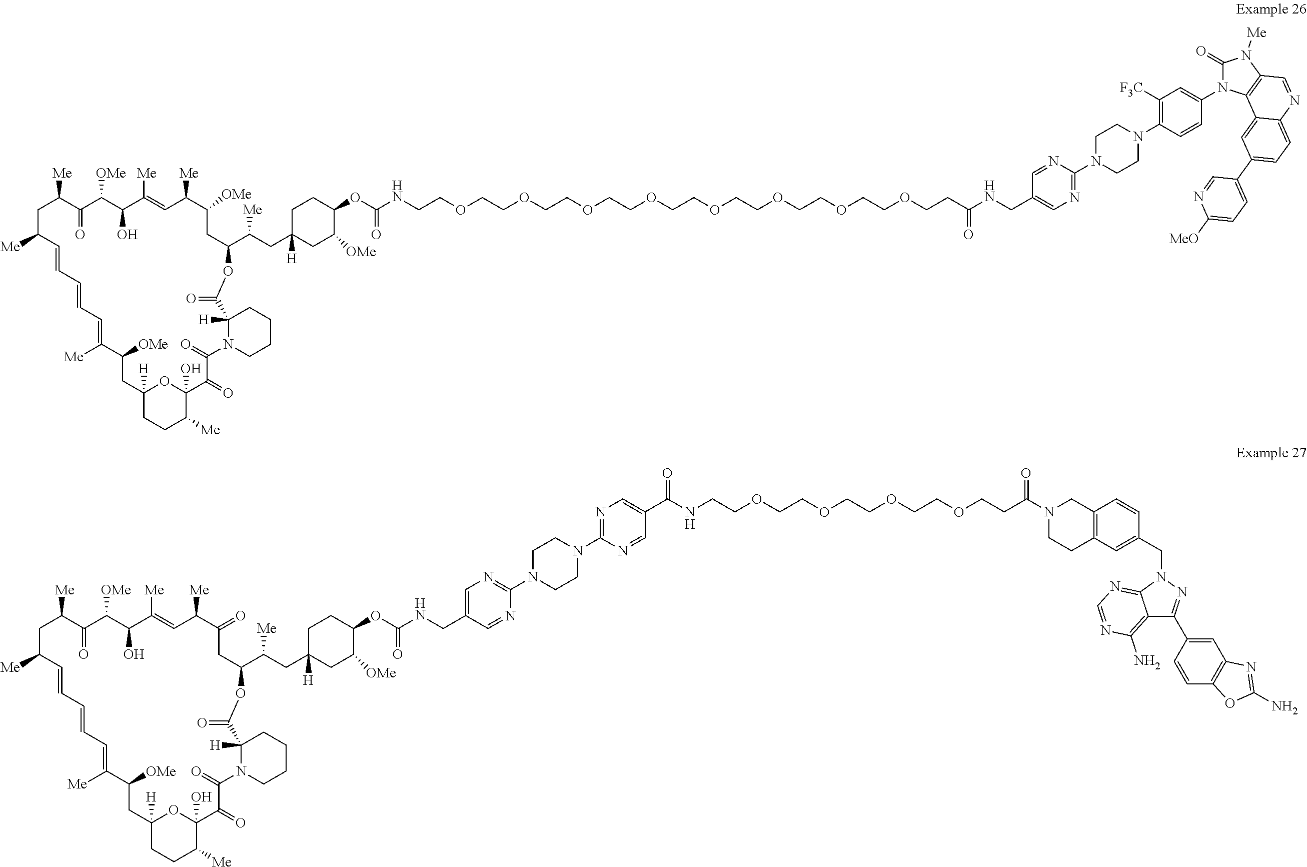

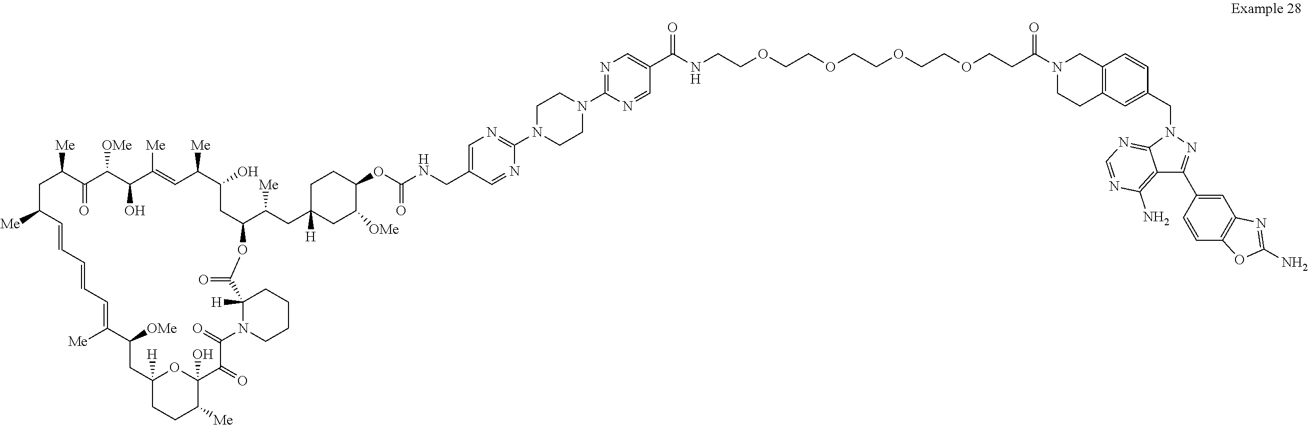 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

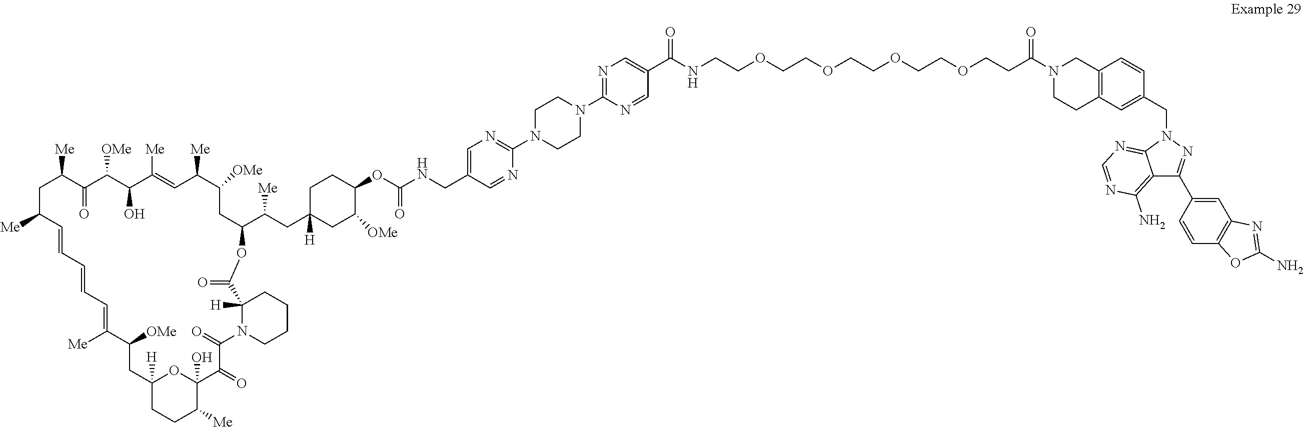 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

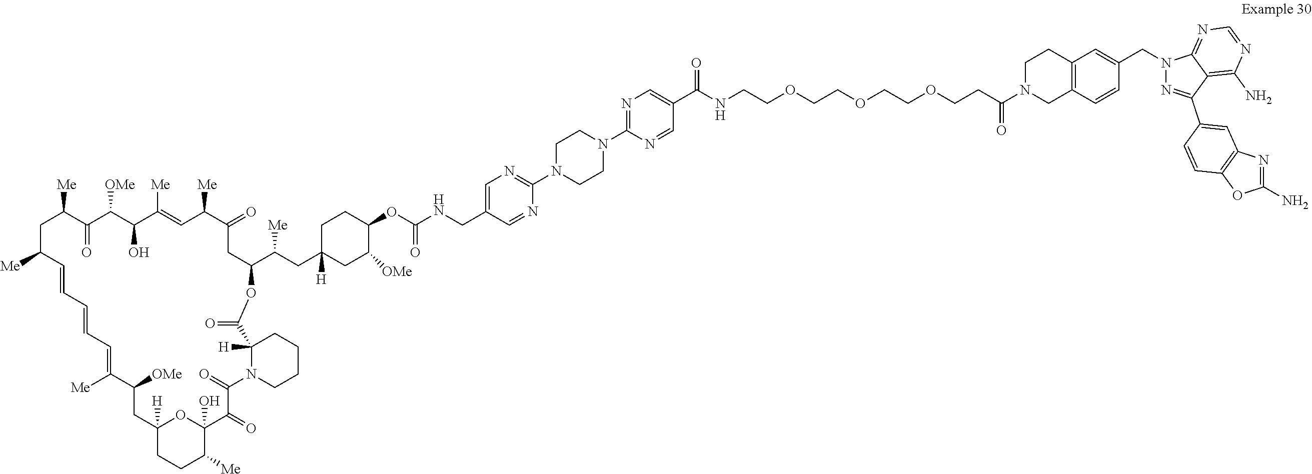 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

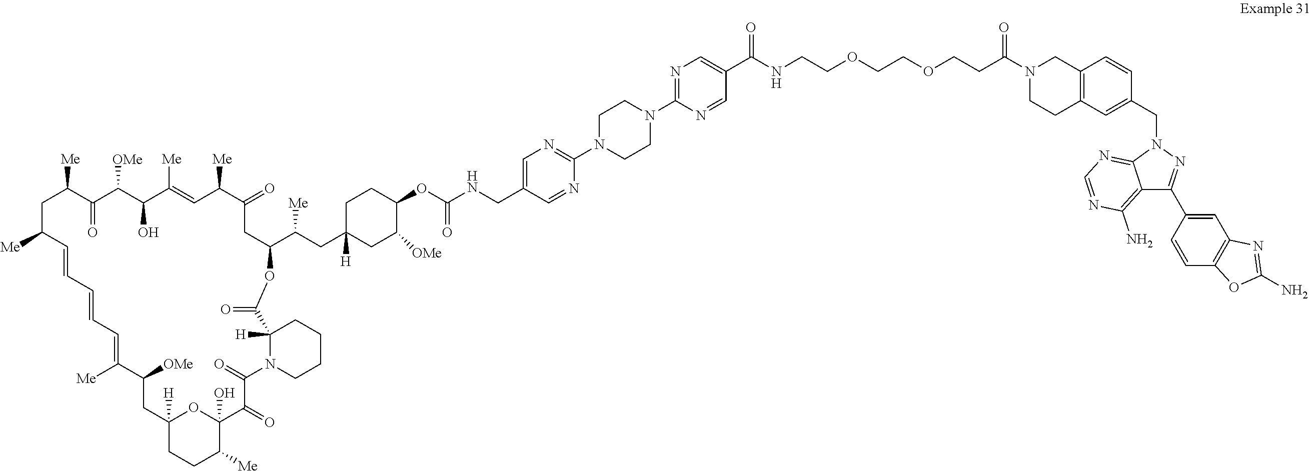 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

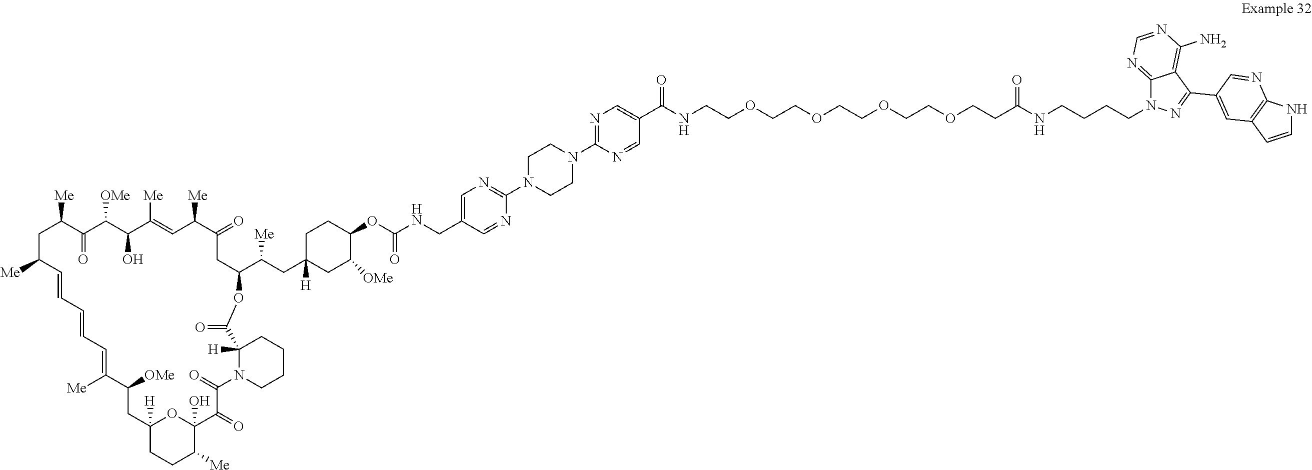 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

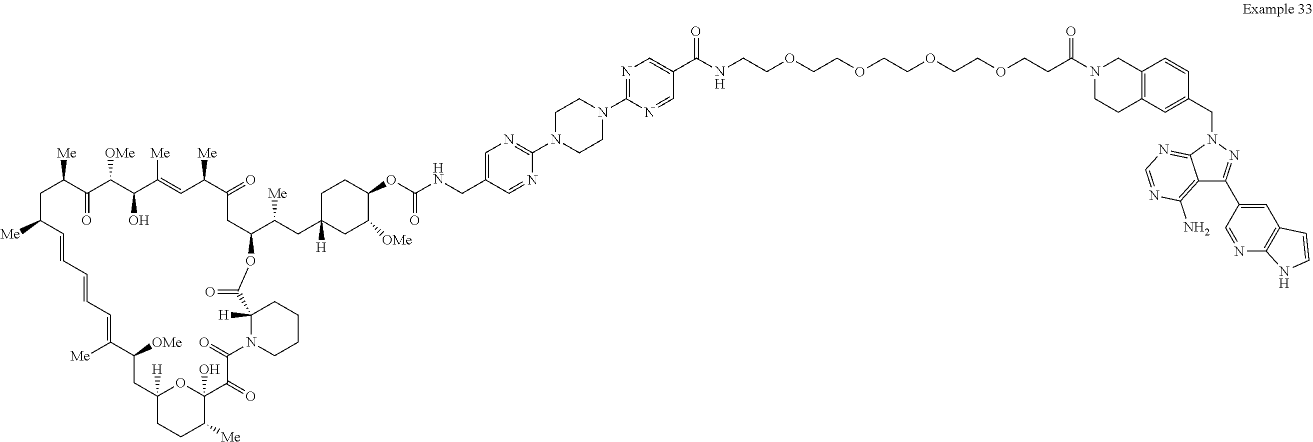 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

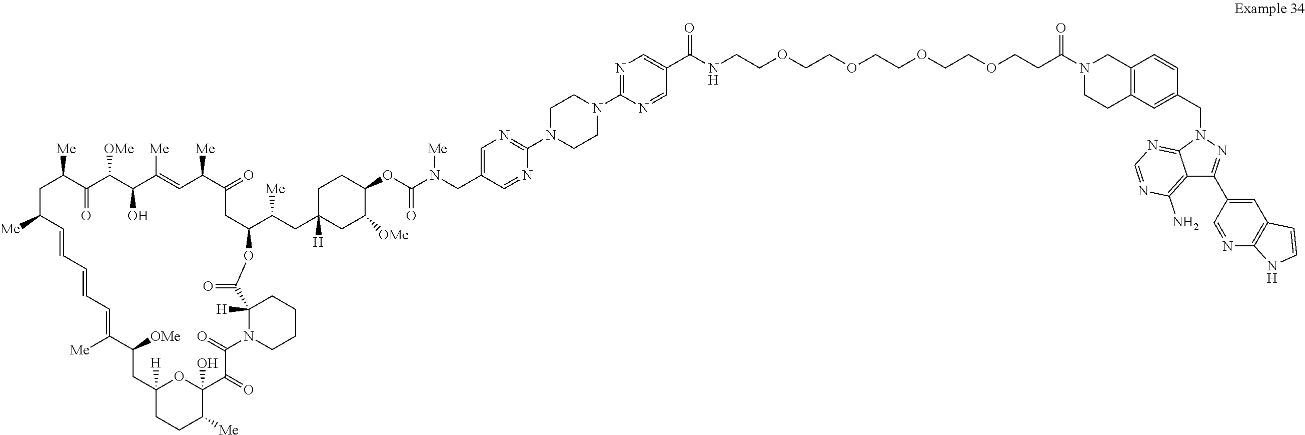 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

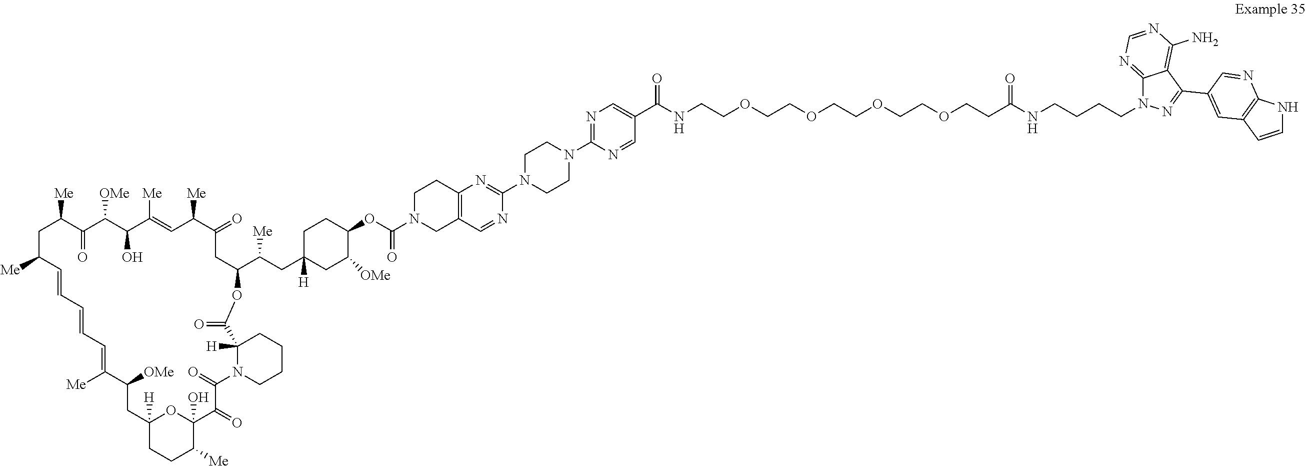 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

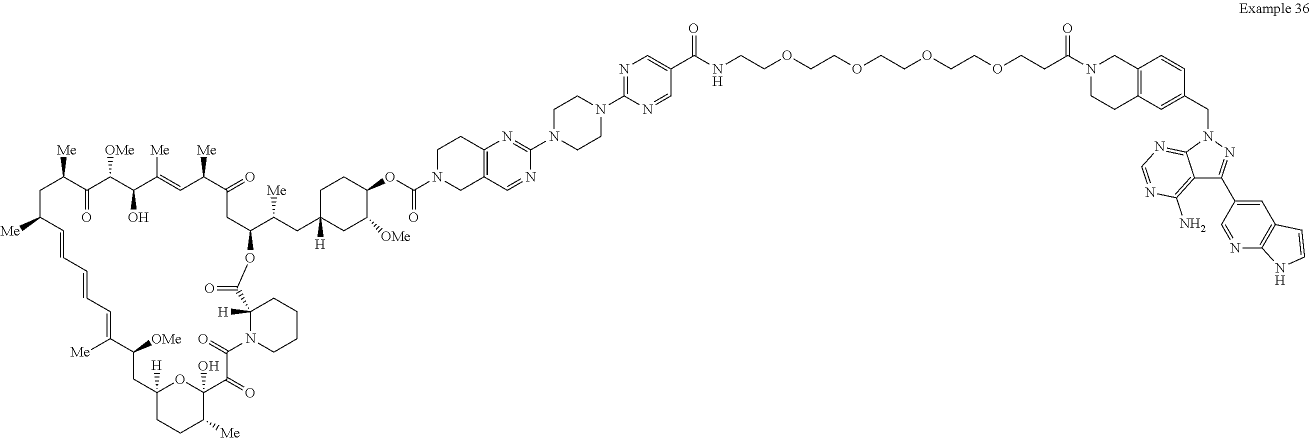 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

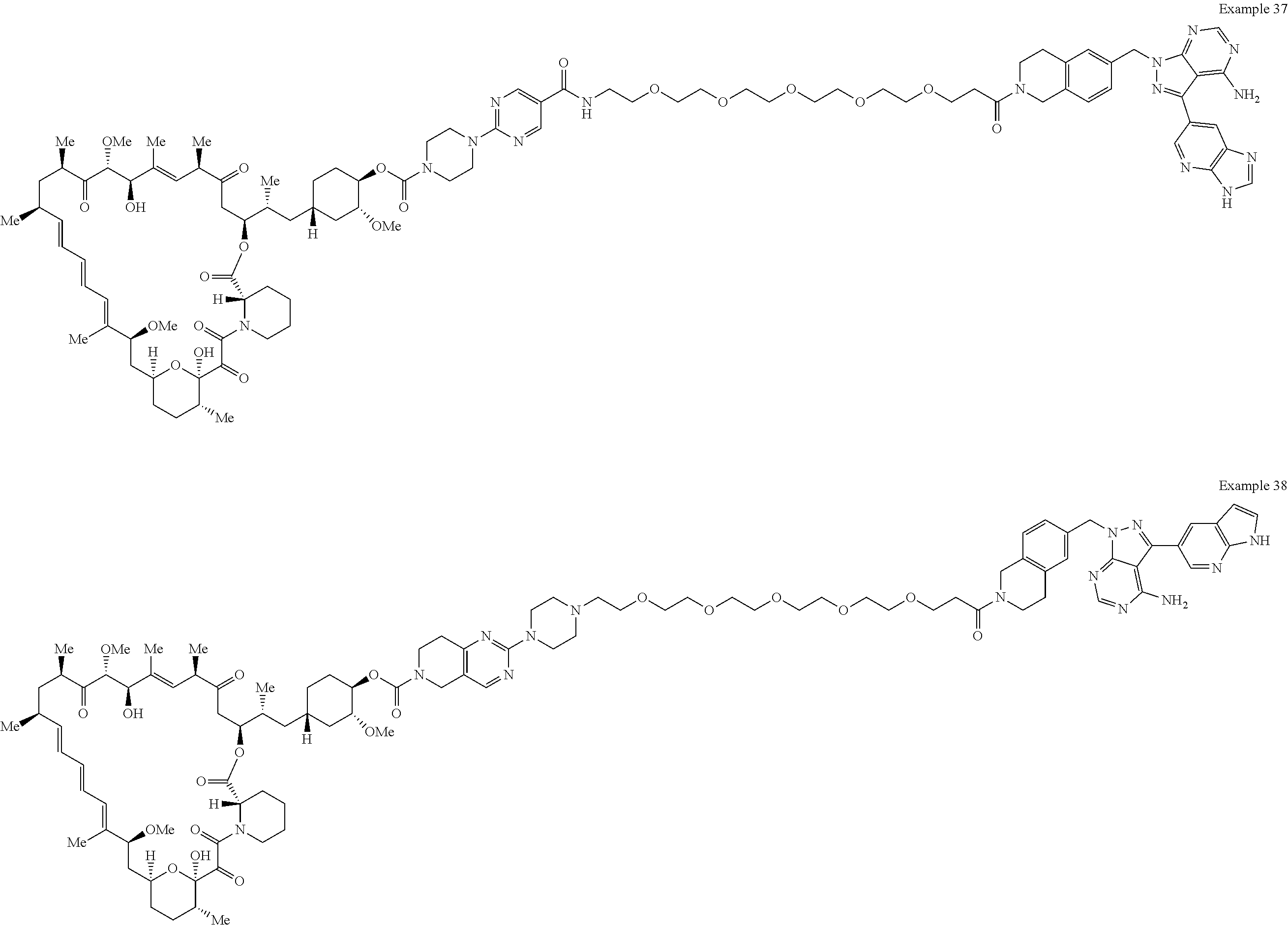

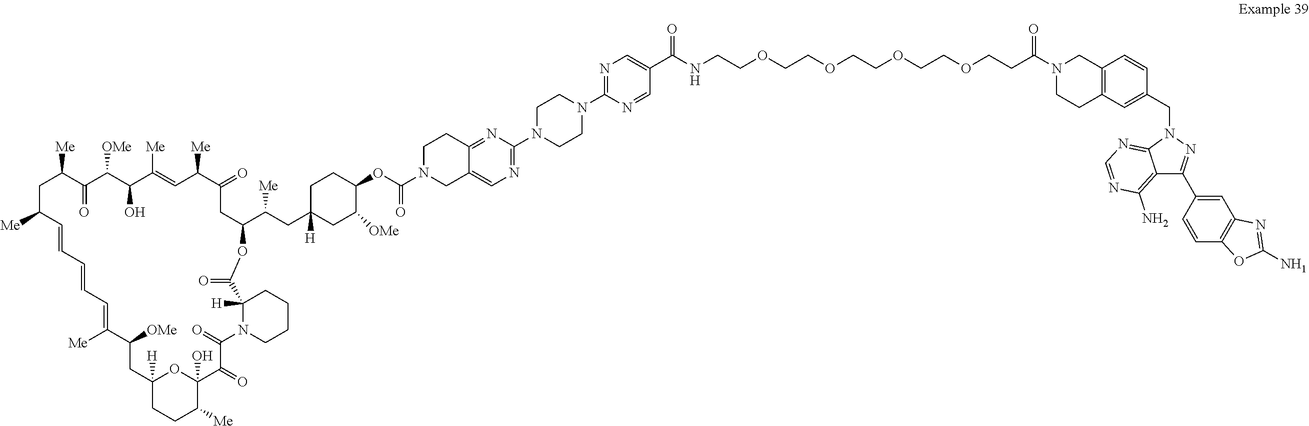 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

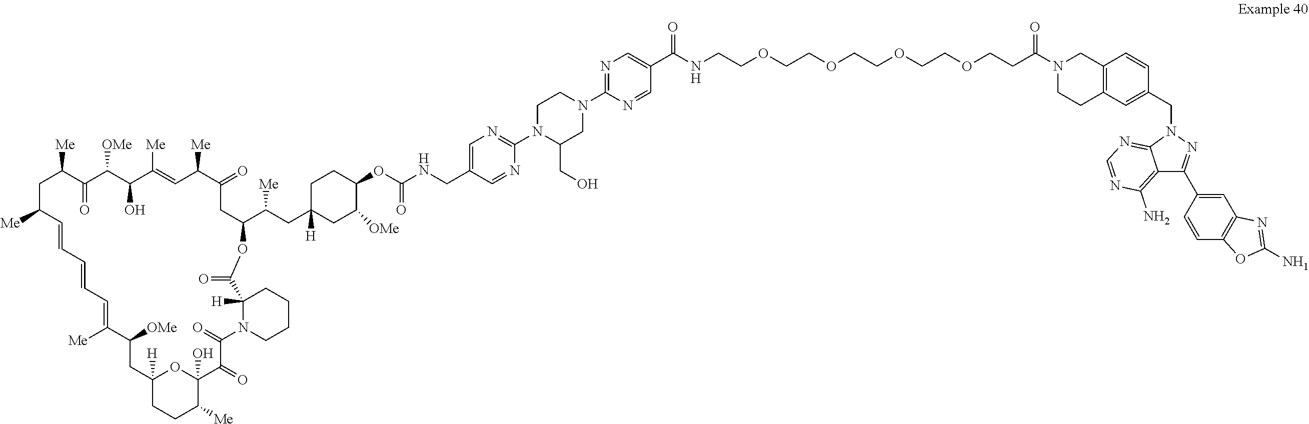 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

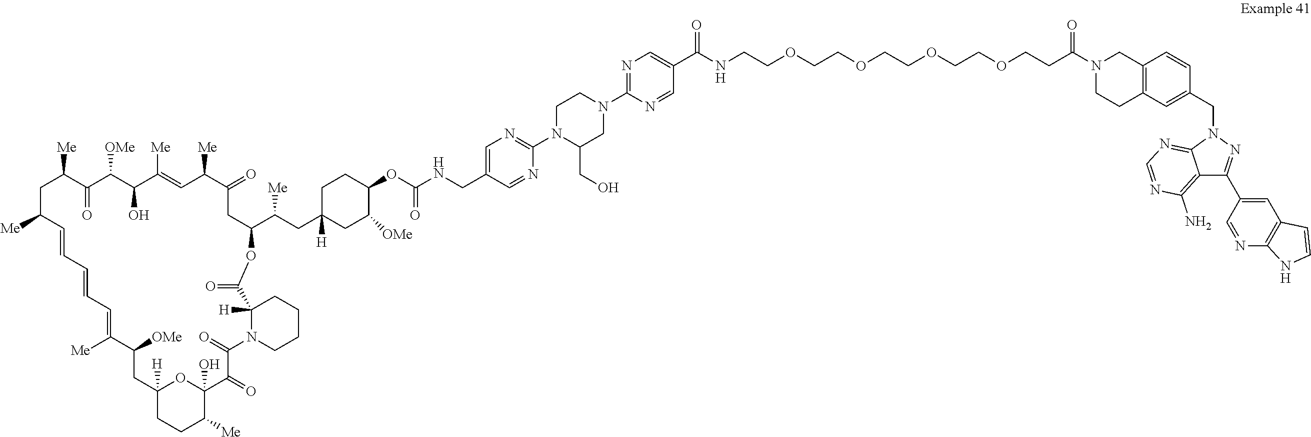 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

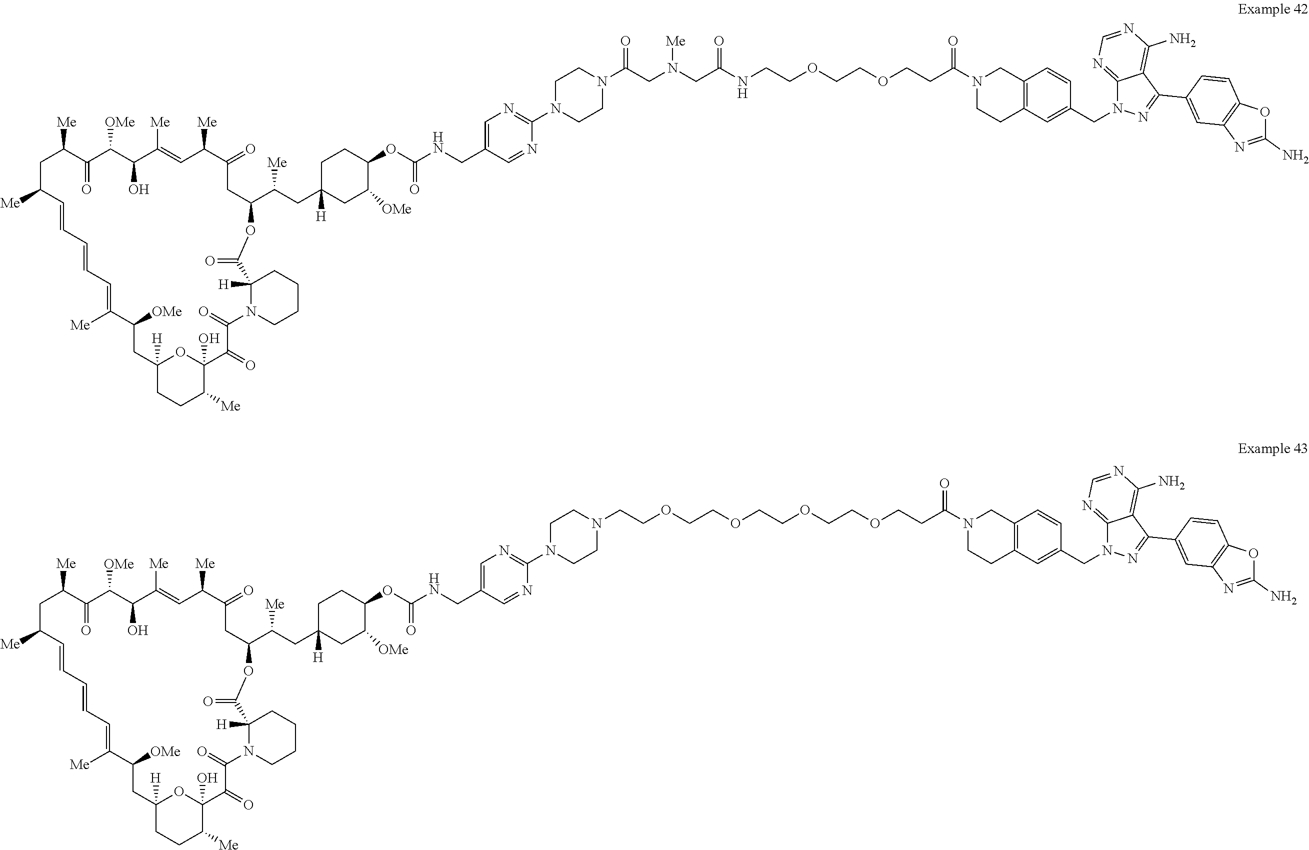

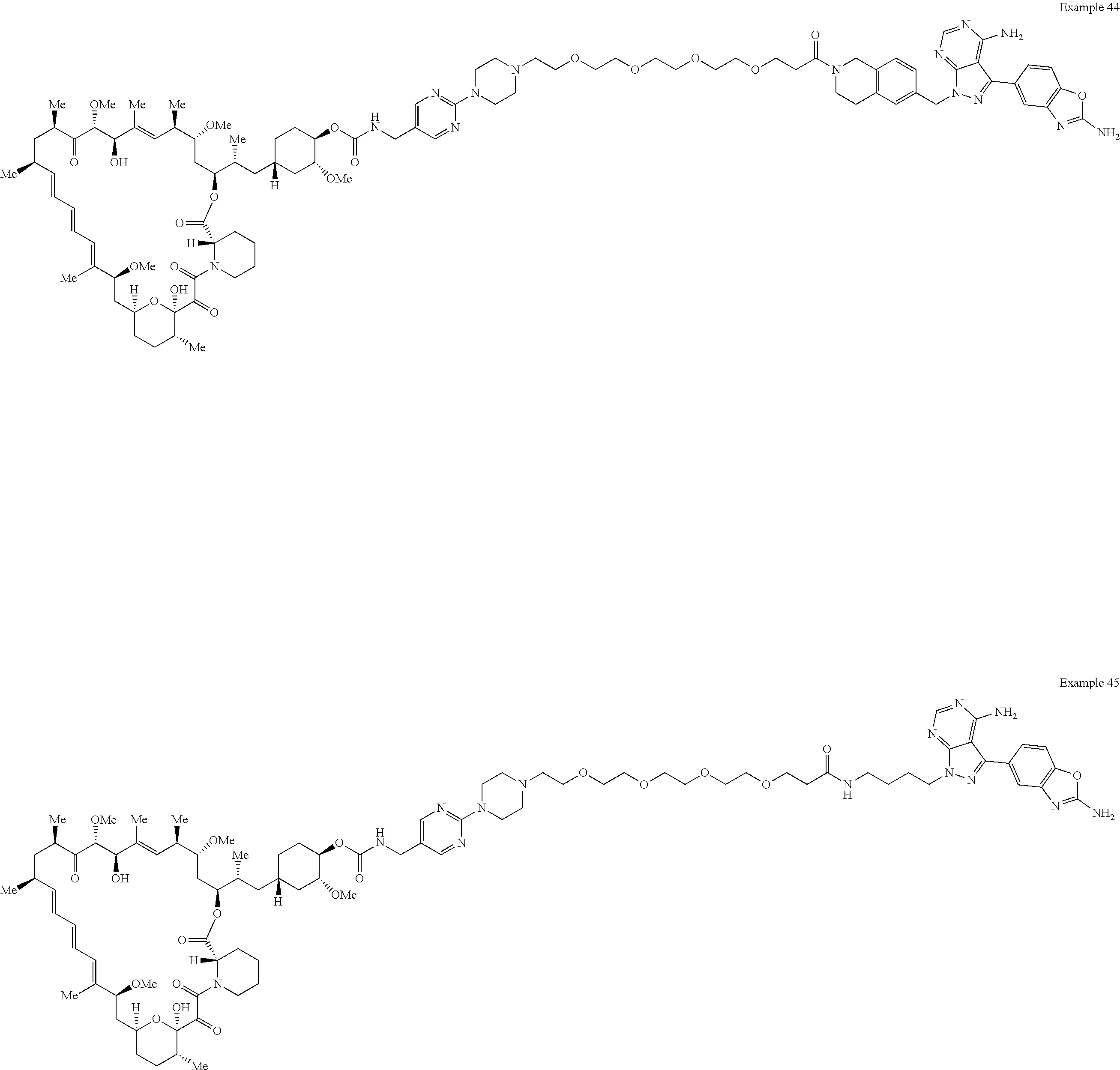

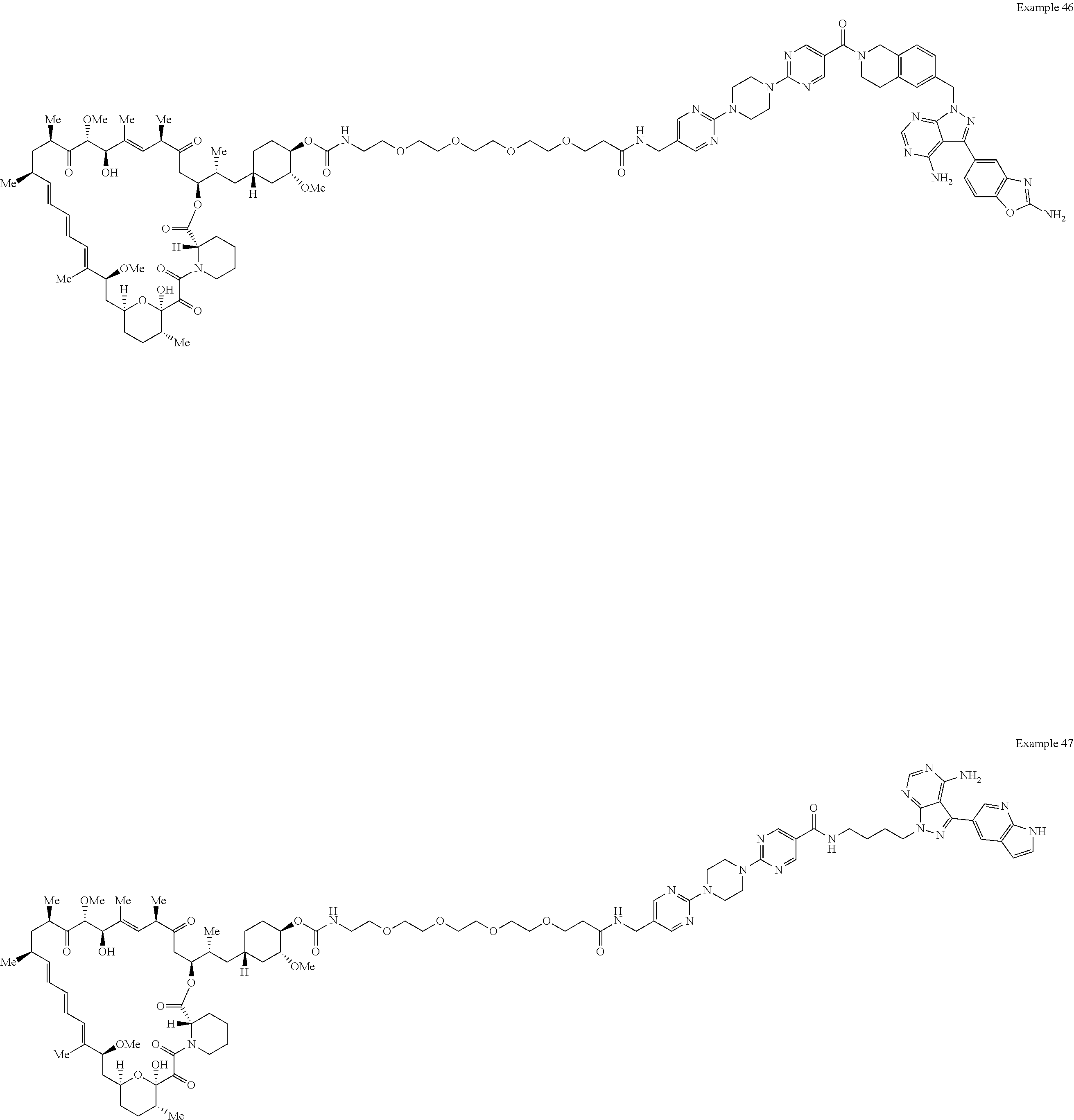

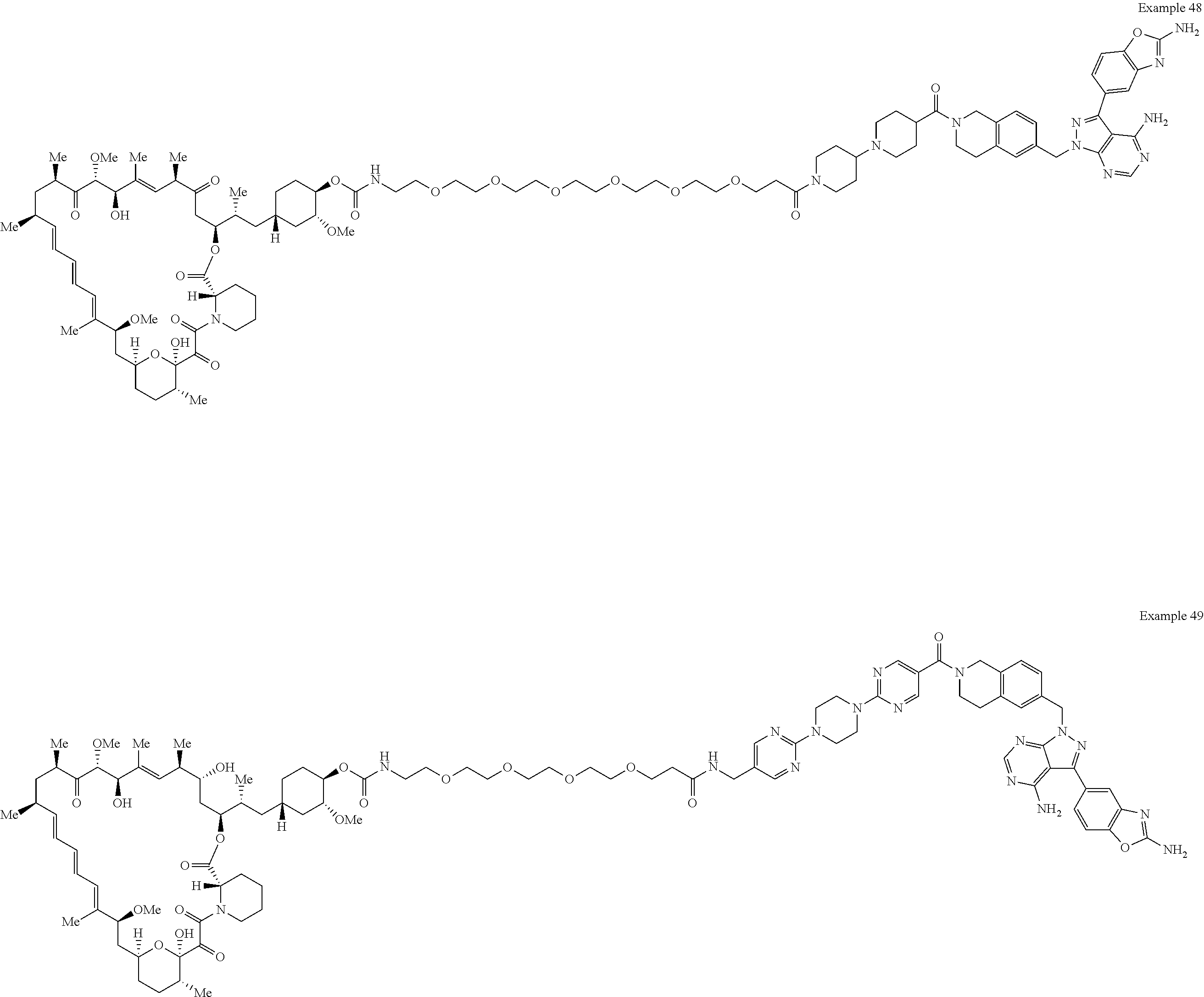

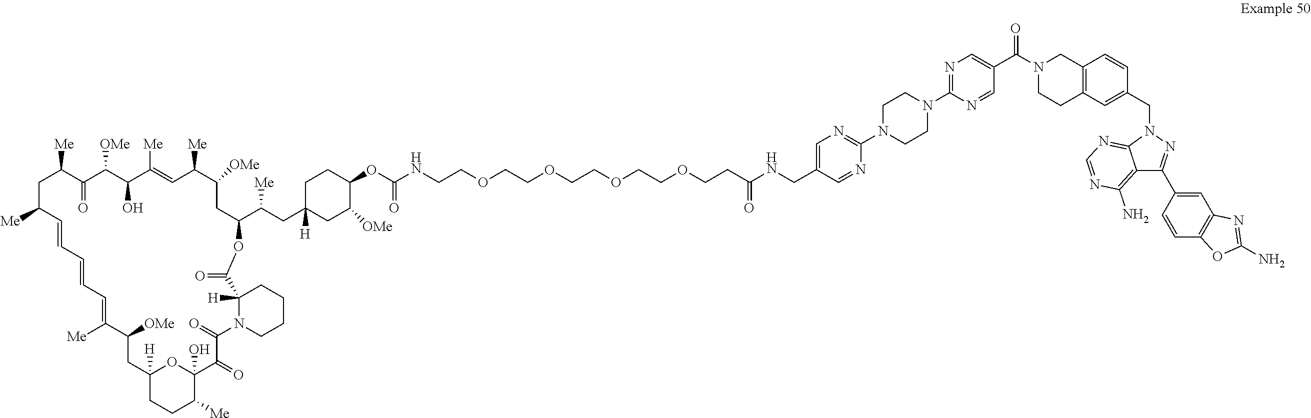 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

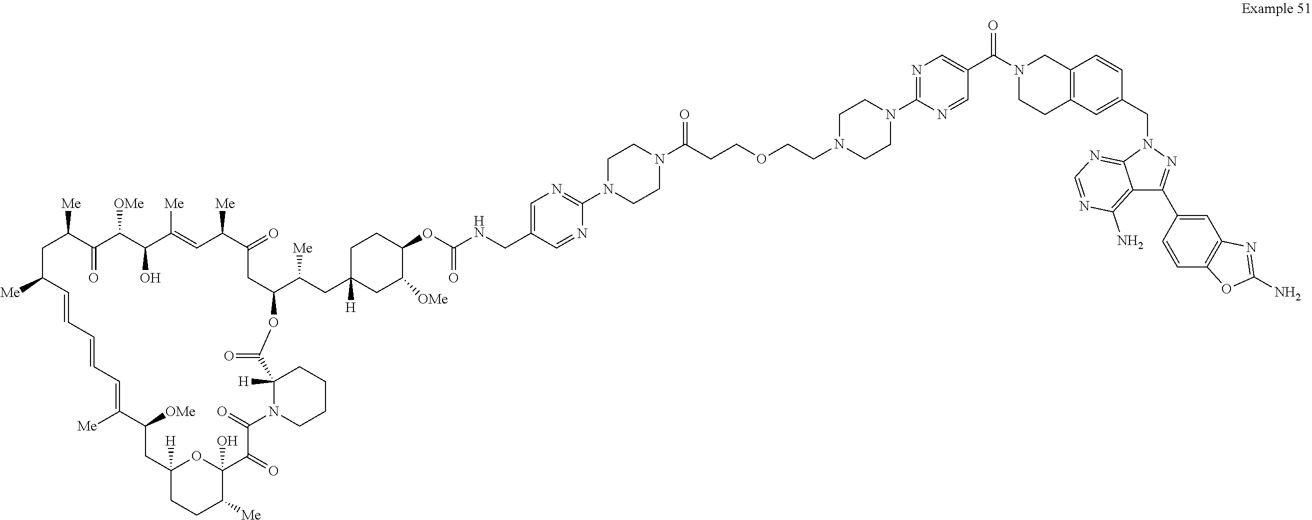 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

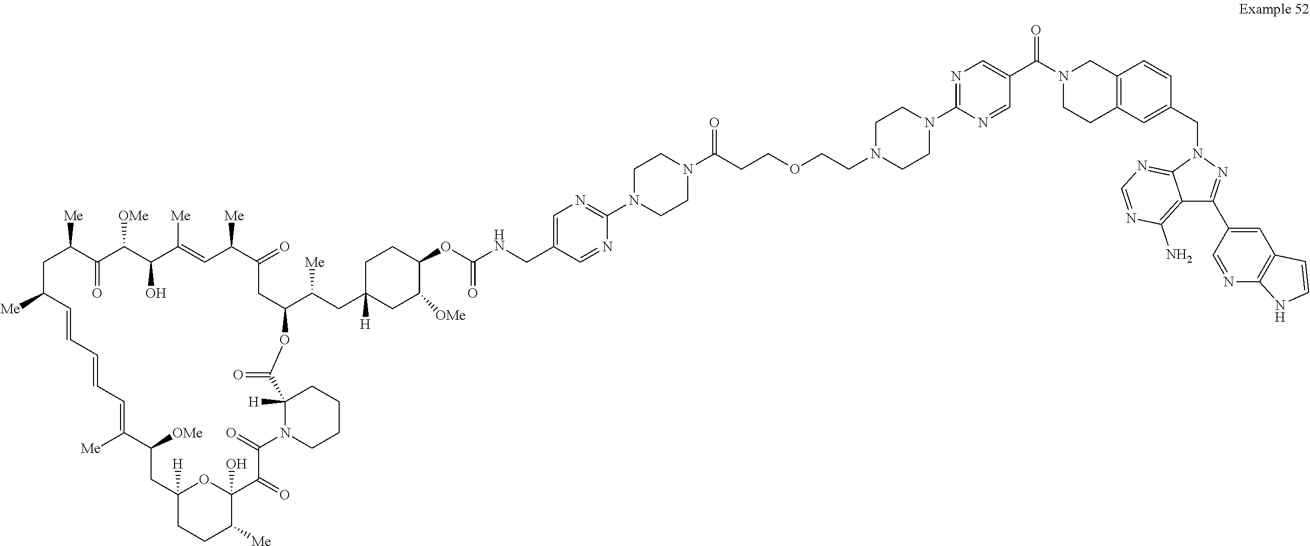 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

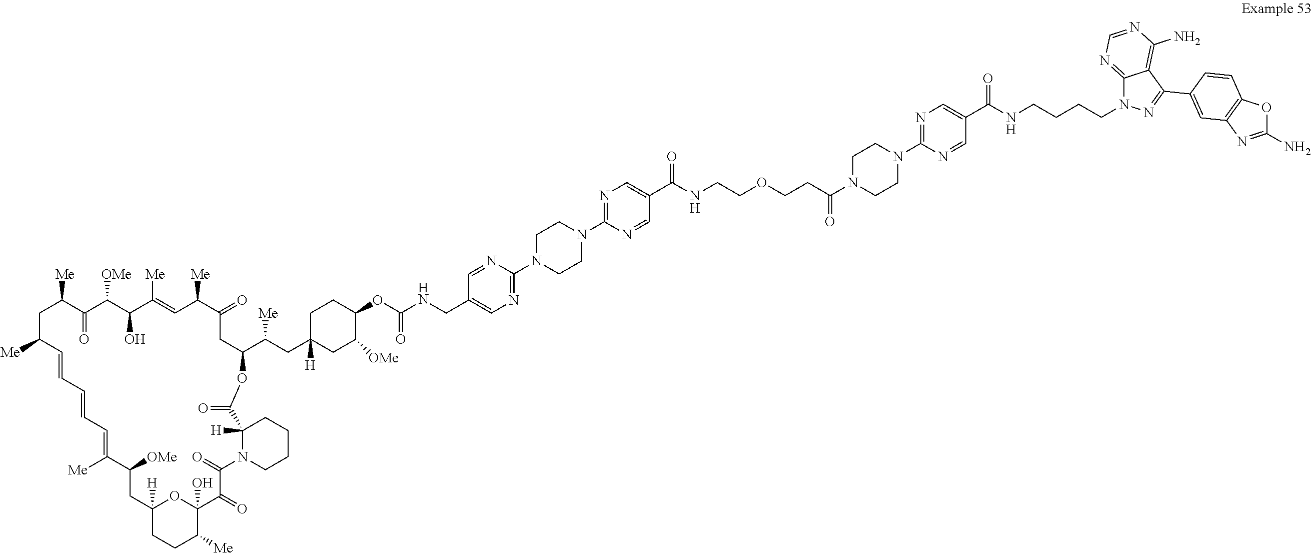 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCC(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

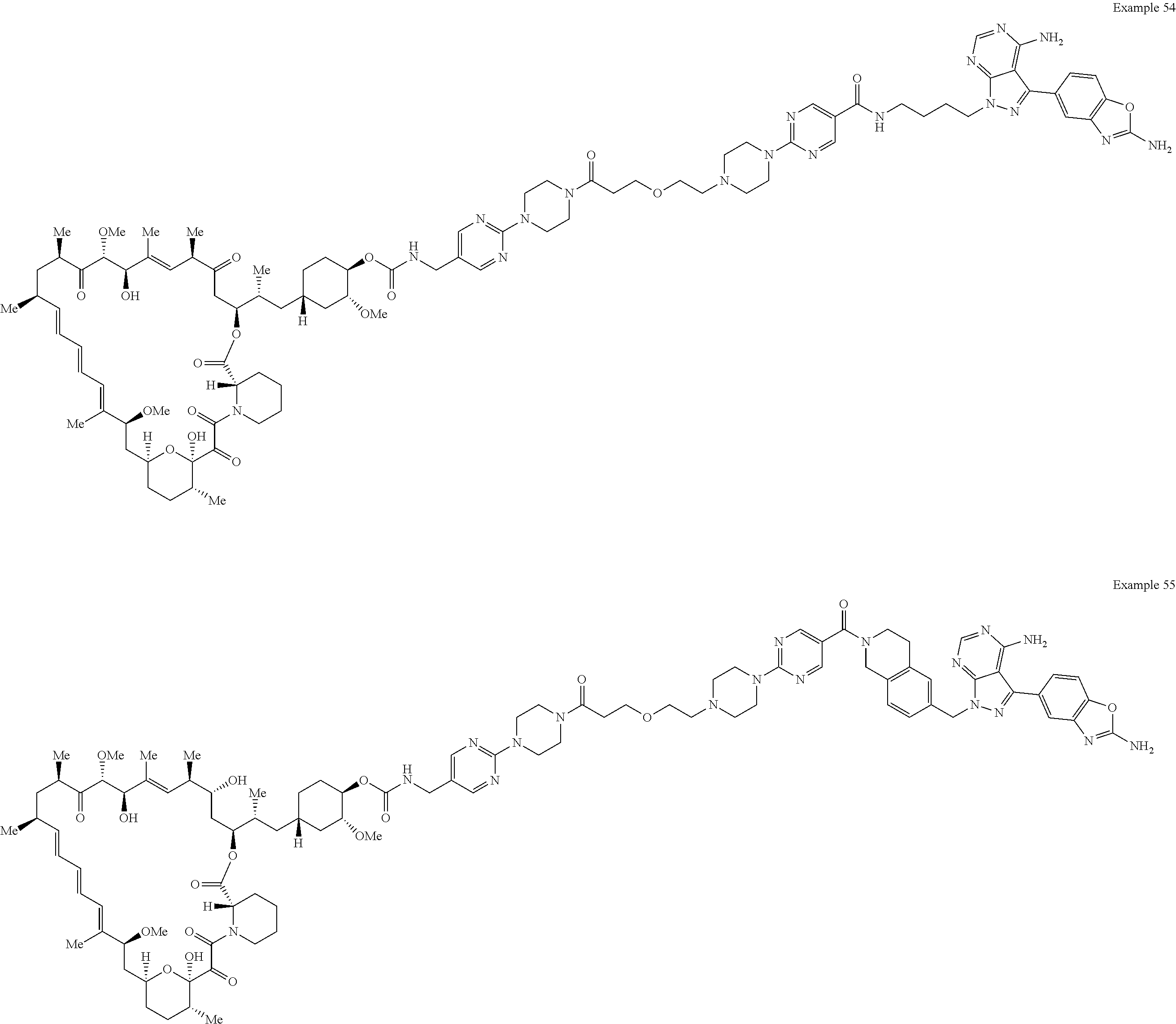

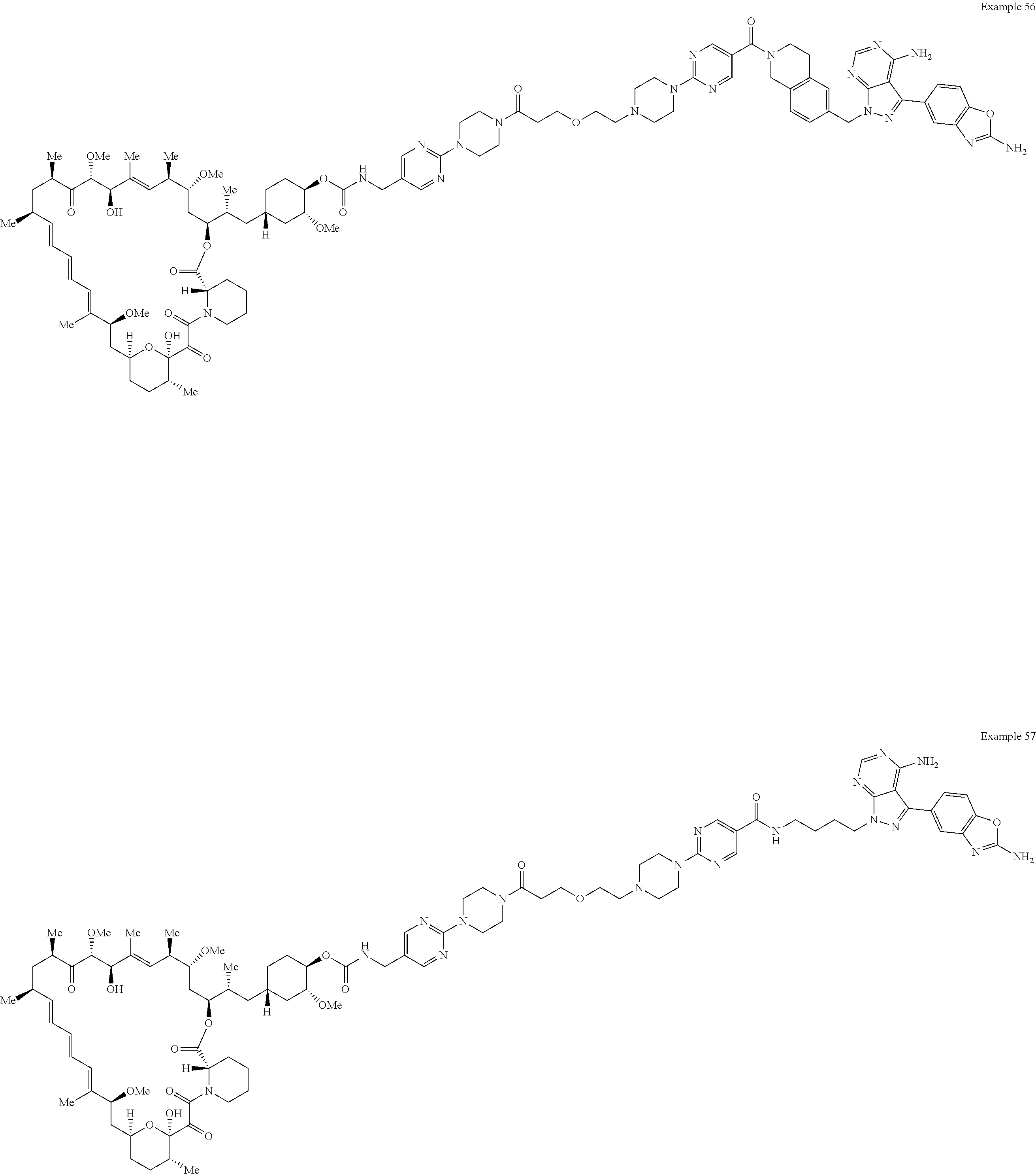

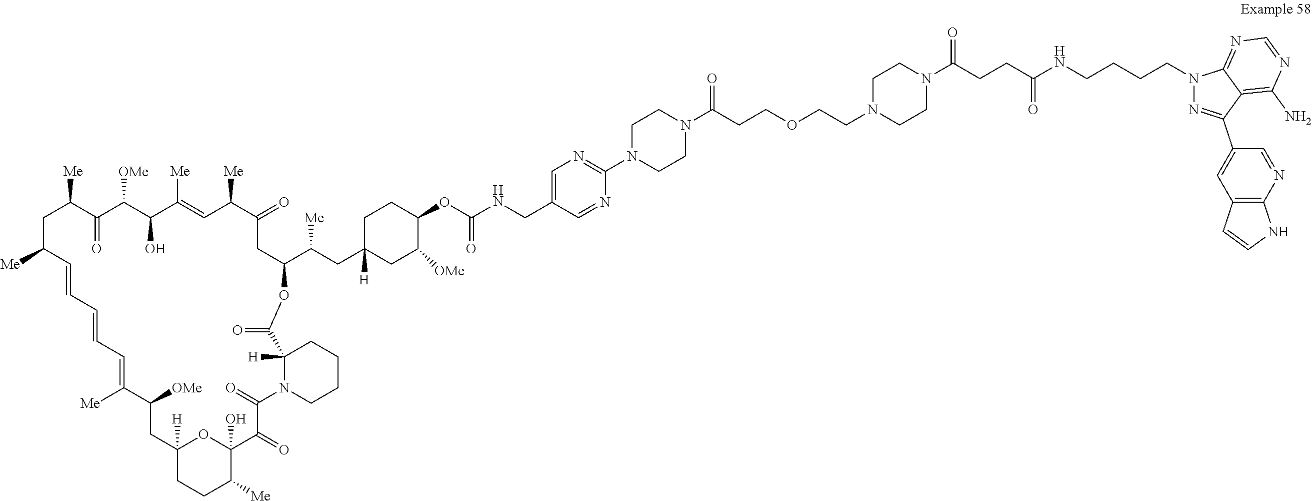 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)NCCCCn6nc(-c7cnc8[nH]ccc8c7)c7c(N)ncnc76)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

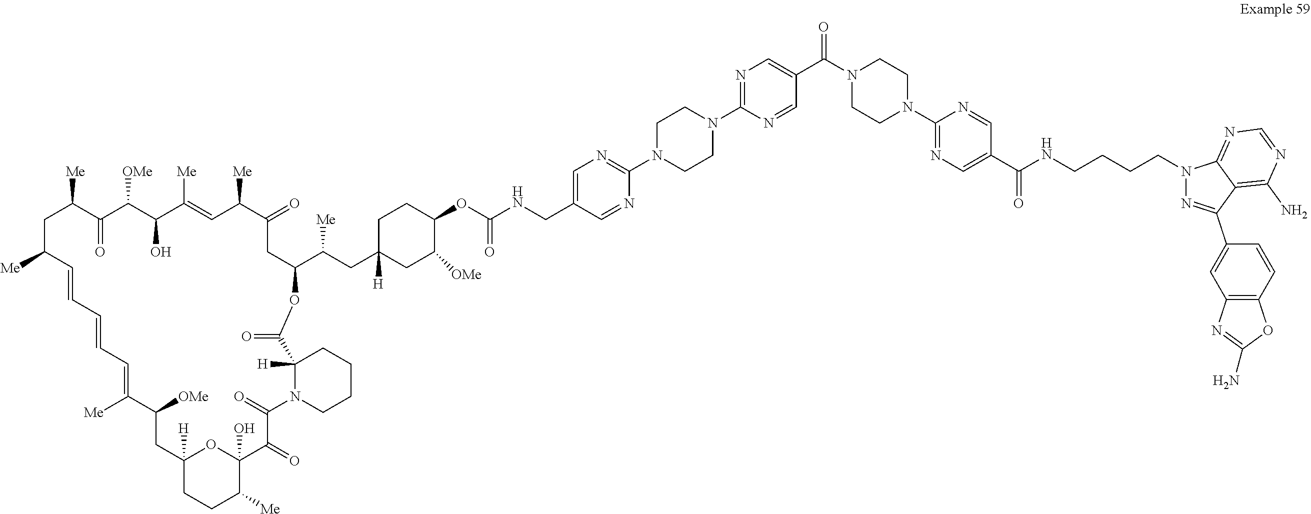 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

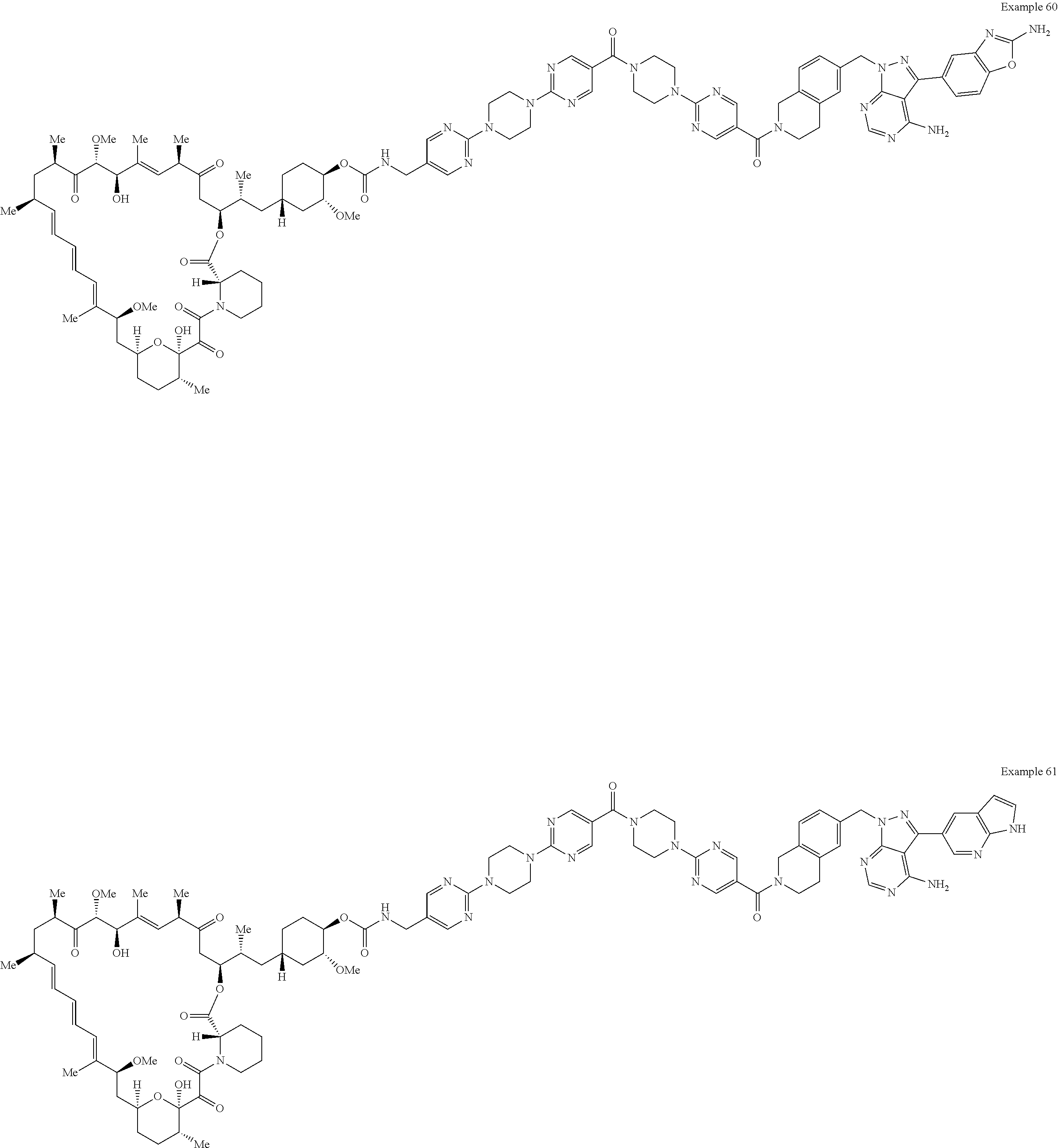

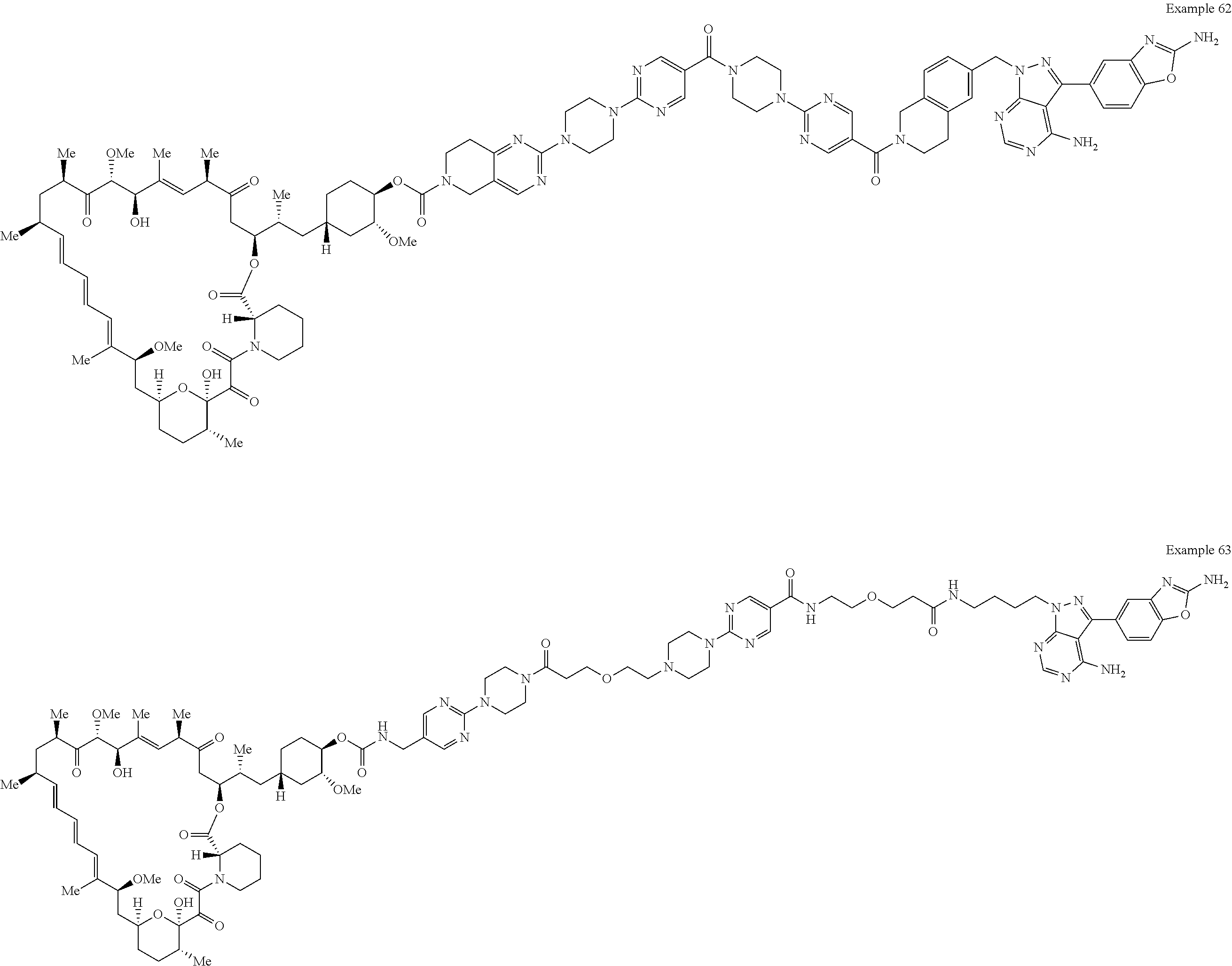

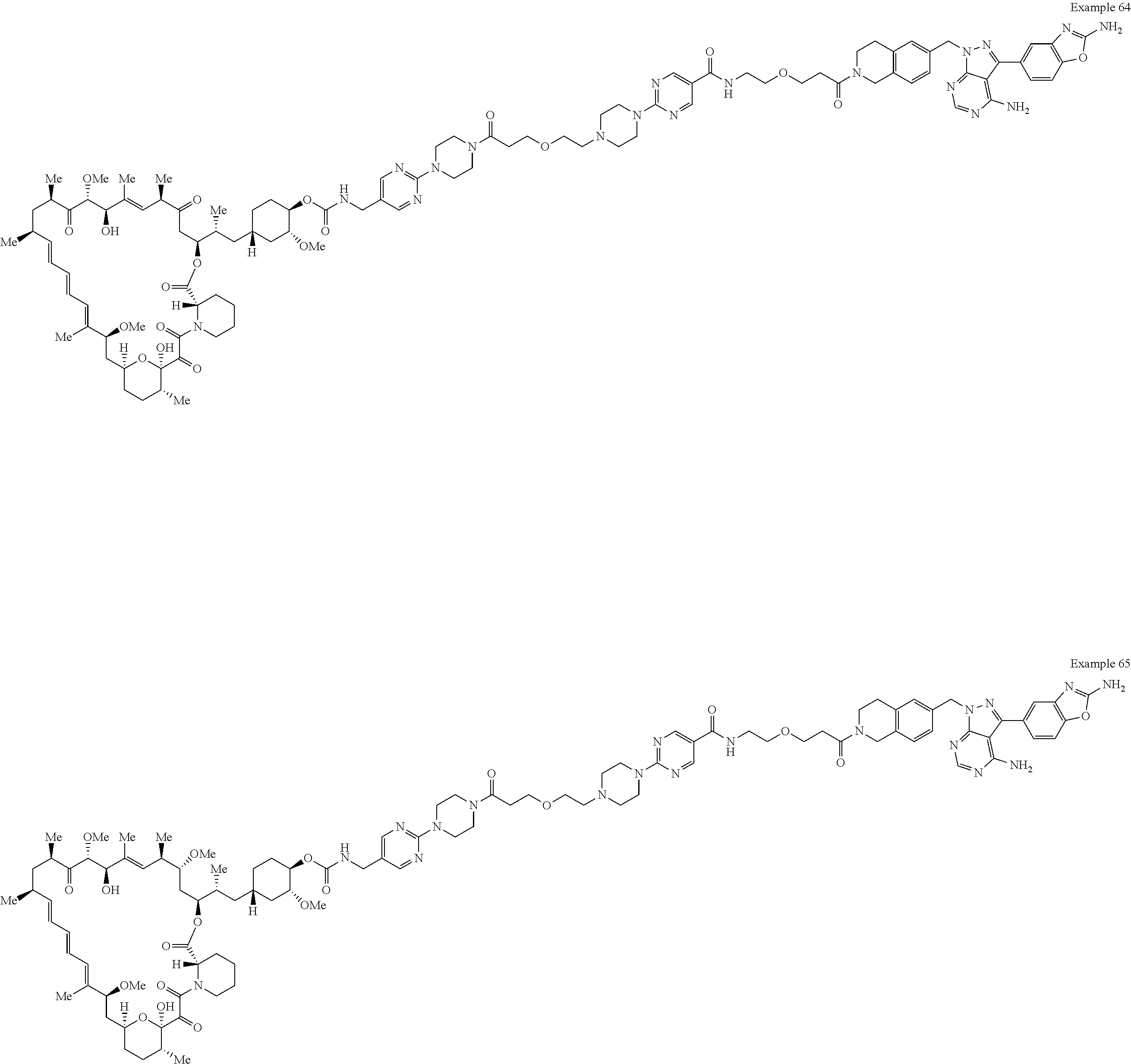

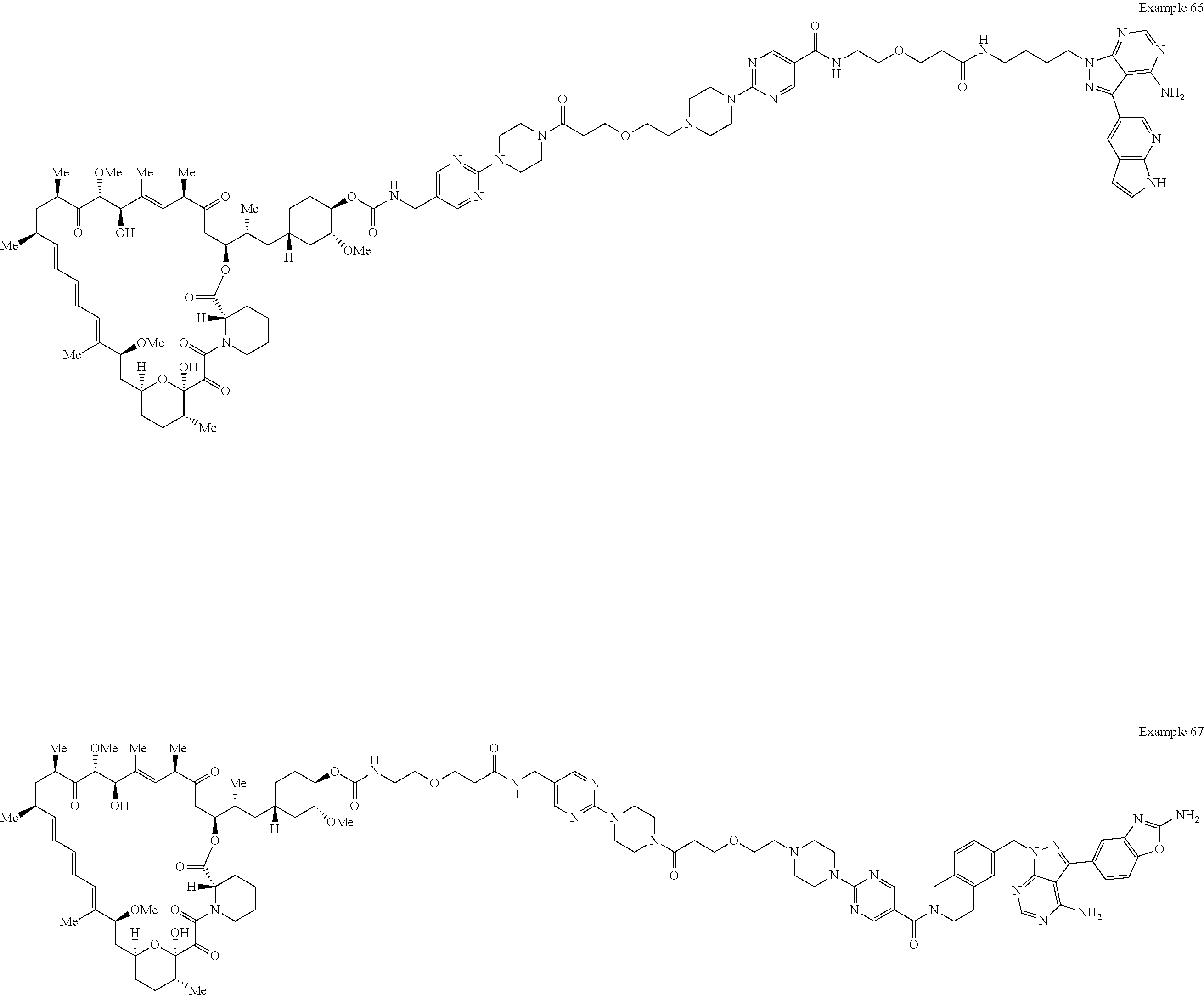

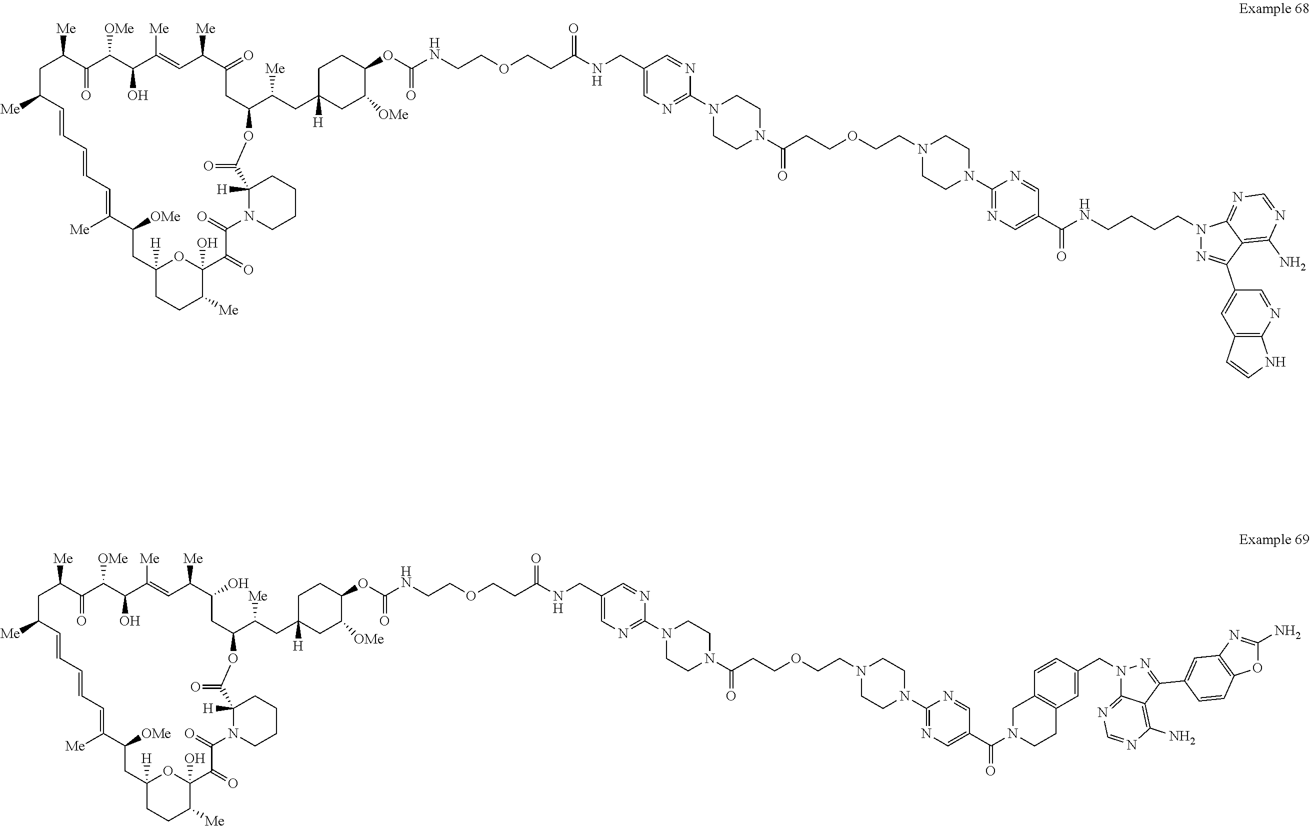

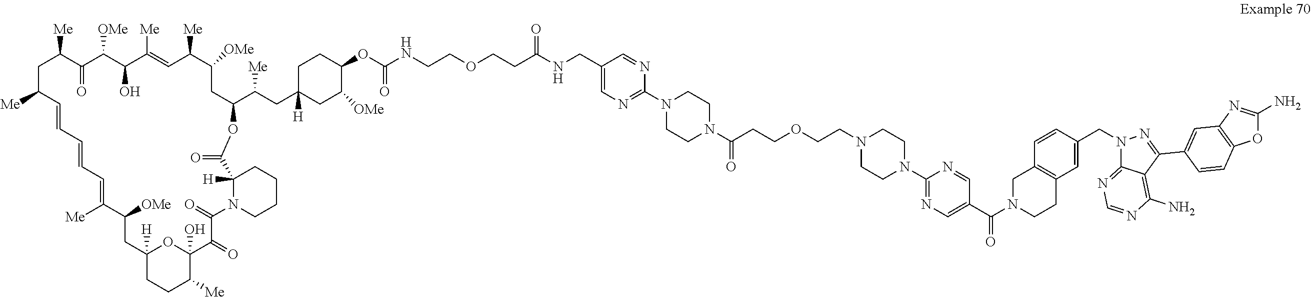 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

or a pharmaceutically acceptable salt or tautomer thereof.

Embodiment I-61. A pharmaceutical compostions composing a compound of any one of Embodiments I-1 to I-60, or a pharmaceutically acceptable salt thereof, and at least one of a pharmaceutically acceptable carrier, diluent, or excipient.

Embodiment I-62. A method of treating a disease or disorder mediated by mTOR comprising administering to the subject suffering from or susceptible to developing a disease or disorder mediated by mTOR a therapeutically effective amount of one or more compounds of any one of Embodiments I-1 to I-60, or a pharmaceutically acceptable salt thereof.

Embodiment 1-63. A method of preventing a disease or disorder mediated by mTOR comprising administering to the subject suffering from or susceptible to developing a disease or disorder mediated by mTOR a therapeutically effective amount of one or more compounds of any one of Embodiments I-1 to I-60, or a pharmaceutically acceptable salt thereof.

Embodiment I-64. A method of reducing the risk of a disease or disorder mediated by mTOR comprising administering to the subject suffering from or susceptible to developing a disease or disorder mediated by mTOR a therapeutically effective amount of one or more compounds of any one of Embodiments I-1 to I-60, or a pharmaceutically acceptable salt thereof.

Embodiment I-65. The method of any one of Embodiments I-62 to I-64, wherein the disease is cancer or an immune-mediated disease.

Embodiment I-66. The method of Embodiment I-65, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment I-67. The method of Embodiment I-65, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment I-68. A method of treating cancer comprising administering to the subject a therapeutically effective amount of one or more compounds of any one of Embodiments I-1 to I-60, or a pharmaceutically acceptable salt thereof.

Embodiment I-69. The method of Embodiment I-68, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment I-70. A method of treating an immune-mediated disease comprising administering to the subject a therapeutically effective amount of one or more compounds of any one of Embodiments I-1 to I-60, or a pharmaceutically acceptable salt thereof.

Embodiment I-71. The method of Embodiment I-70, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment I-72. A method of treating an age related condition comprising administering to the subject a therapeutically effective amount of one or more compounds of any one of Embodiments I-1 to I-60, or a pharmaceutically acceptable salt thereof.

Embodiment I-73. The method of Embodiment I-72, wherein the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

Embodiment I-74. A compound of any one of Embodiments I-1 to I-60, or a pharmaceutically acceptable salt thereof, for use in treating, preventing, or reducing the risk of a disease or condition mediated by mTOR.

Embodiment I-75. Use of a compound of any of Embodiments I-1 to I-60, or a pharmaceutically acceptable salt thereof, in the manufacture of a medicament for treating, preventing, or reducing the risk of a disease or disorder mediated by mTOR.

Embodiment I-76. A compound of any one of Embodiments I-1 to I-60, or a pharmaceutically acceptable salt thereof, for use in treating cancer.

Embodiment I-77. Use of a compound of any one of Embodiments I-1 to I-60, or a pharmaceutically acceptable salt thereof, in the manufacture of a medicament for treating cancer.

Embodiment I-78. A compound of any one of Embodiments I-1 to I-60, or a pharmaceutically acceptable salt thereof, for use in treating an immune-mediated disease.

Embodiment I-79. Use of a compound of any one of Embodiments I-1 to I-60, or a pharmaceutically acceptable salt thereof, in the manufacture of a medicament for treating an immune-mediated disease.

Embodiment I-80. A compound of any one of Embodiments I-1 to I-60, or a pharmaceutically acceptable salt thereof, for use in treating an age related condition.

Embodiment I-81. Use of a compound of any one of Embodiments I-1 to I-60, or a pharmaceutically acceptable salt thereof, in the manufacture of a medicament for treating an age related condition.

Some embodiments of this disclosure are Embodiment II, as follows:

Embodiment II-1. A compound of Formula Ic:

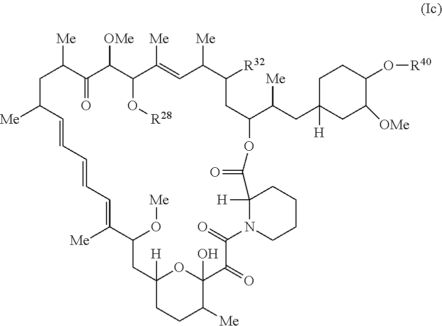

or a pharmaceutically acceptable salt or tautomer thereof, wherein:

R³² is —H, ═O, —OR³, —N₃, or —O—C(═Z¹)—R^{32a};

R²⁸ is —H, (C₁-C₆)alkyl, or —C(═Z¹)—R^{28a};

R⁴⁰ is —H or —C(═Z¹)—R^{40a};
wherein when R²⁸ and R⁴⁰ are H, then R³² is not ═O;

each Z¹ is independently O or S;

R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;

A¹ and A² are independently absent or are independently selected from

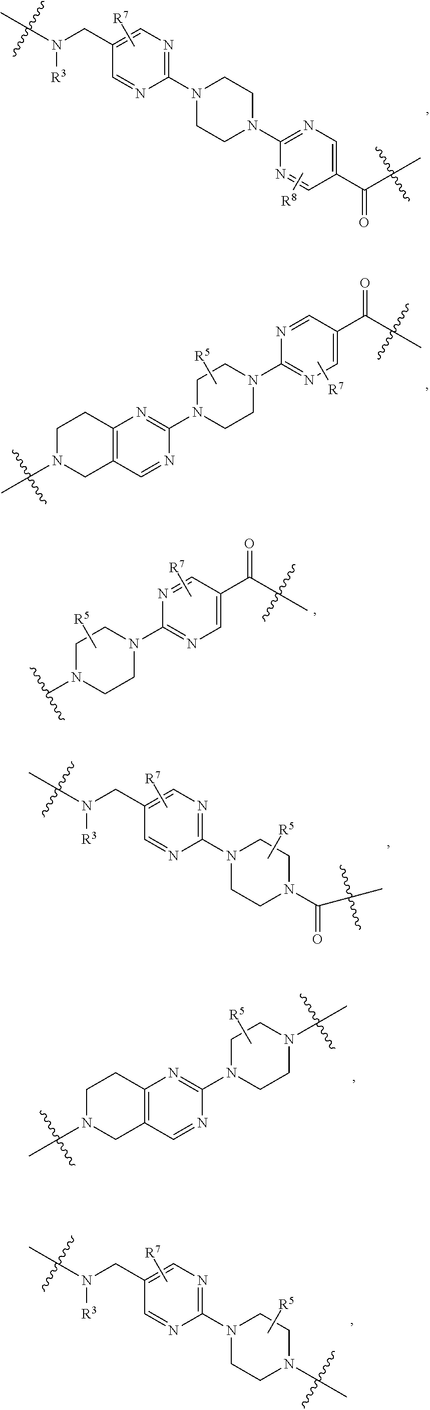

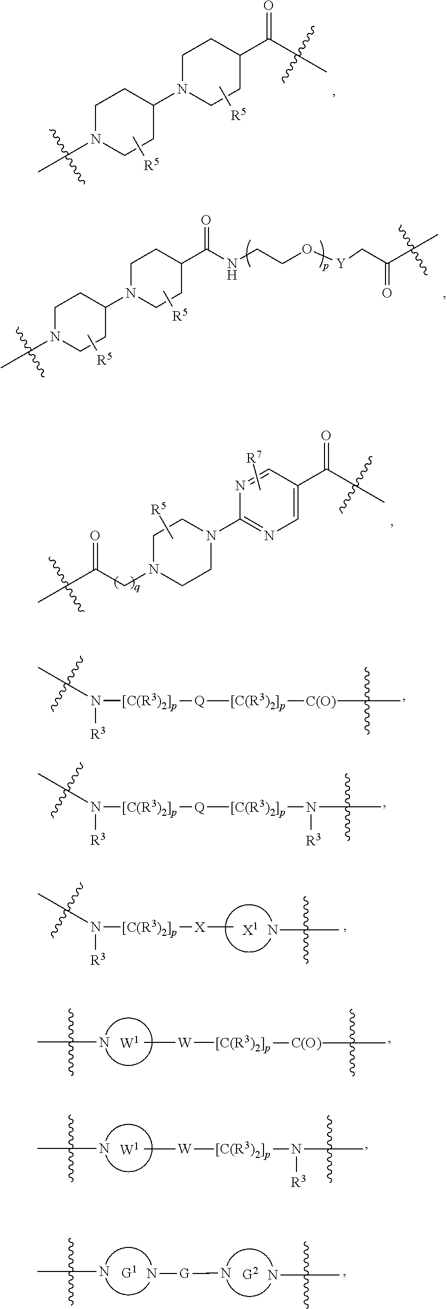

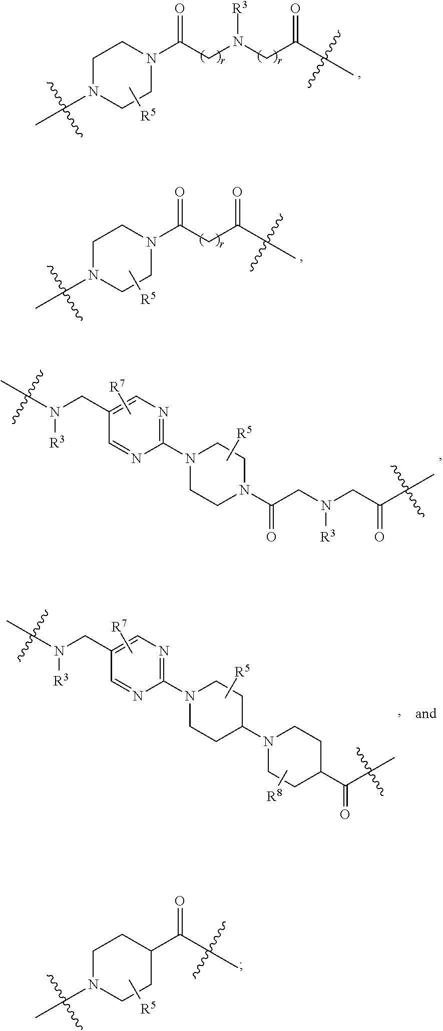

wherein the bond on the left side of A¹, as drawn, is bound to —C(═Z¹)— or L²; and wherein the bond on the right side of the A² moiety, as drawn, is bound to B or L³; each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

each L¹ is independently selected from

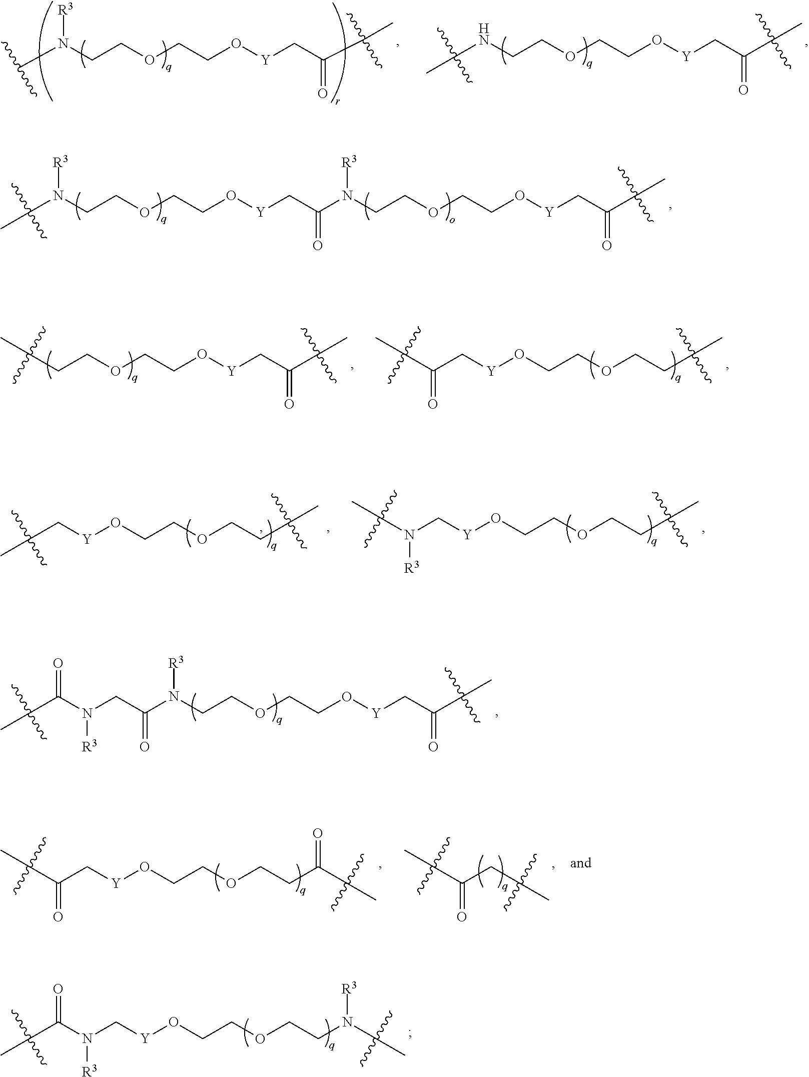

L² and L³ are independently absent or are independently selected from

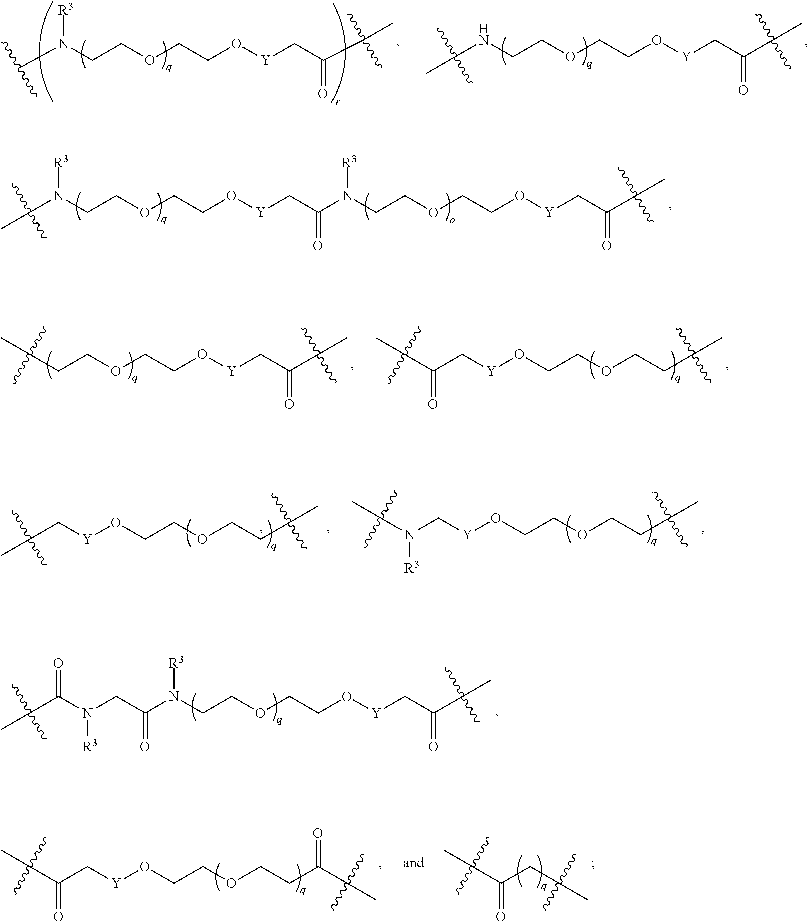

each B is independently selected from

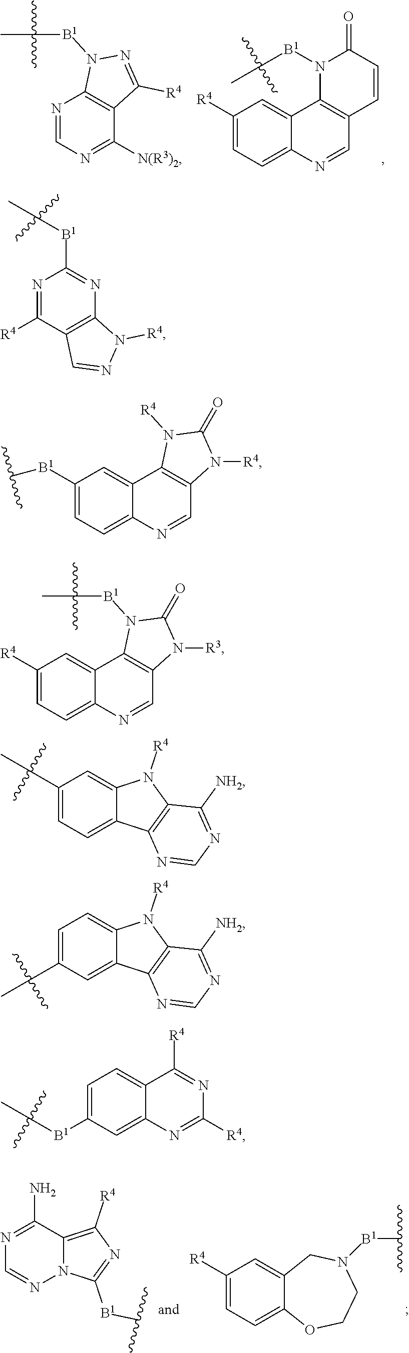

each B¹ is independently selected from

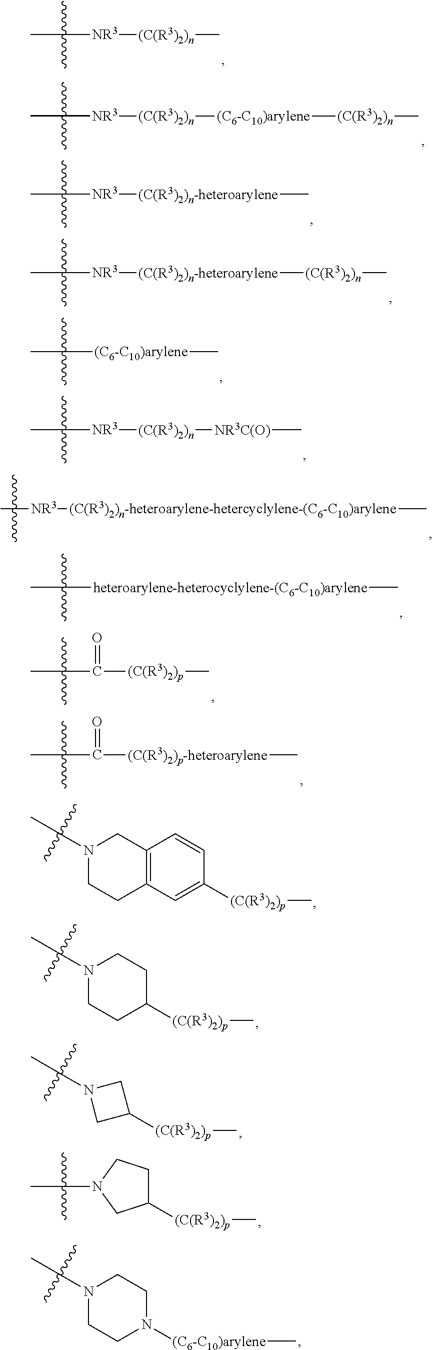

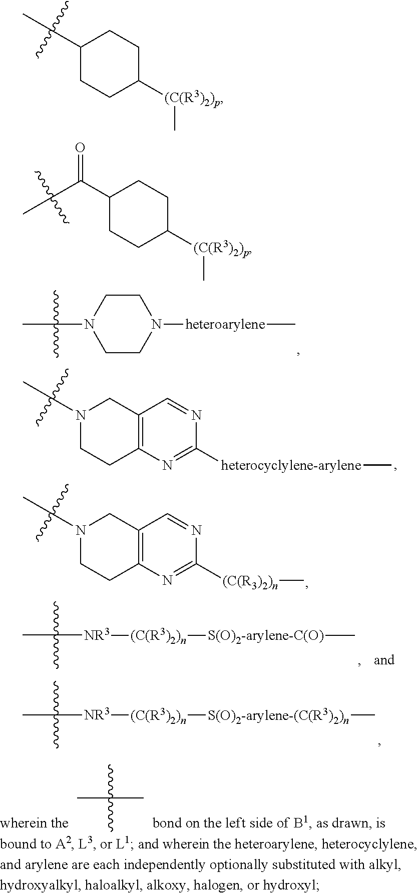

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each R⁵ is independently H, (C₁-C₆)alkyl, —C(O)OR³, or —N(R³)₂, wherein the alkyl of (C₁-C₆)alkyl is optionally substituted with —N(R³)₂ or —OR³;

each R⁶ is independently H, (C₁-C₆)alkyl, —C(O)OR³, or —N(R³)₂, wherein the alkyl of (C₁-C₆)alkyl is optionally substituted with —N(R³)₂ or —OR³;

each R⁷ is independently H, (C₁-C₆)alkyl, —C(O)OR³, or —N(R³)₂, wherein the alkyl of (C₁-C₆)alkyl is optionally substituted with —N(R³)₂ or —OR³;

each R⁸ is independently H, (C₁-C₆)alkyl, —C(O)OR³, or —N(R³)₂, wherein the alkyl of (C₁-C₆)alkyl is optionally substituted with —N(R³)₂ or —OR³;

each Y is independently C(R³)₂ or a bond;

each n is independently an integer from one to 12;

each o is independently an integer from zero to 30;

each p is independently an integer from zero to 12;

each q is independently an integer from zero to 30; and

each r is independently an integer from one to 6.

Embodiment II-1A. A compound of Formula Ia:

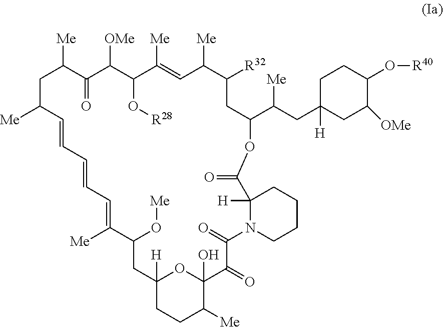

or a pharmaceutically acceptable salt or tautomer thereof, wherein:

R³² is —H, ═O, —OR³, —N₃, or —O—C(═Z¹)—R^{32a};

R²⁸ is —H, (C₁-C₆)alkyl, or —C(═Z¹)—R^{28a};

R⁴⁰ is —H or —C(═Z¹)—R^{40a};
wherein when R²⁸ and R⁴⁰ are H, then R³² is not ═O;

each Z¹ is independently O or S;

R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;

A¹ and A² are independently absent or are independently selected from

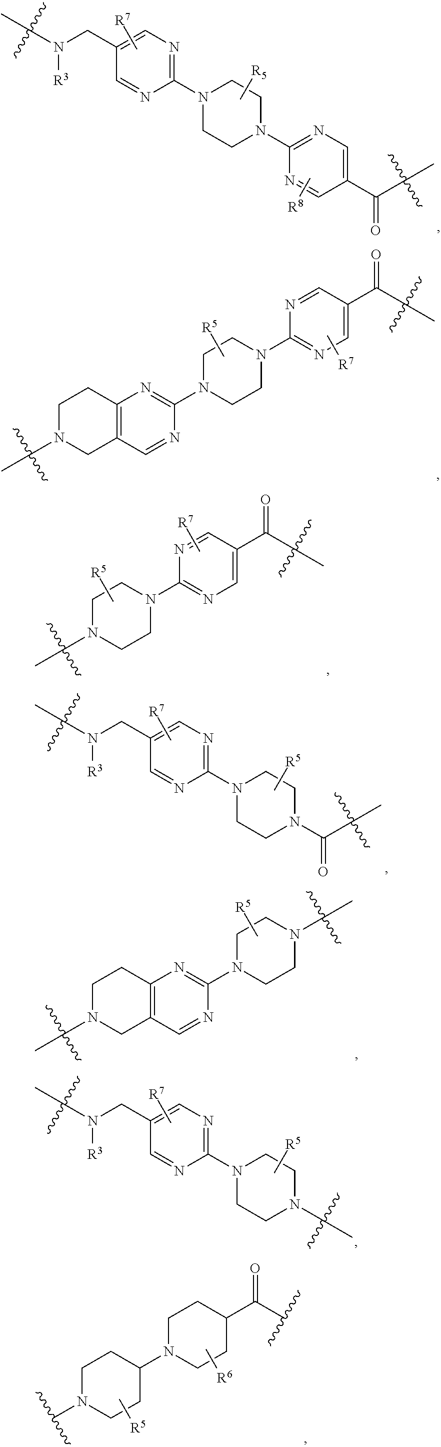

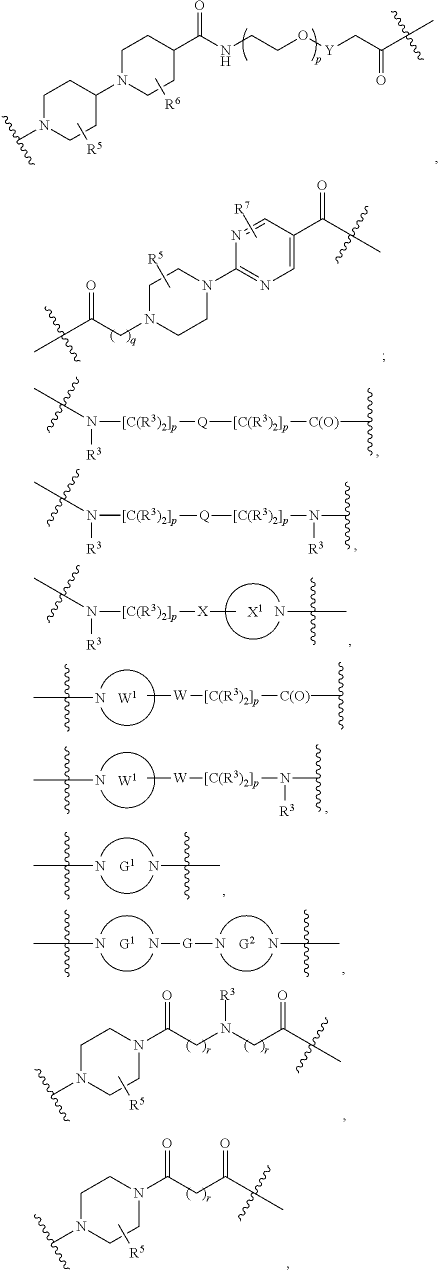

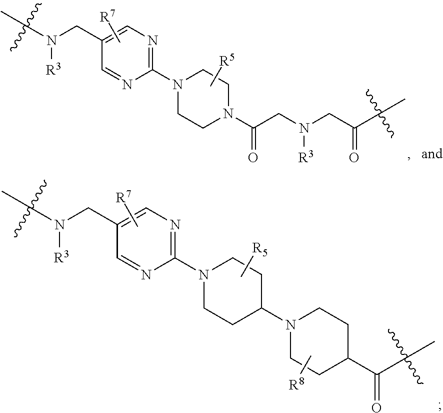

wherein the bond on the left side of A¹, as drawn, is bound to —C(═Z¹)— or L²; and wherein the bond on the right side of the A² moiety, as drawn, is bound to B or L³;

each Q is independently 1 to 3 rings selected from arylene, cycloalkylene, heteroarylene, and heterocyclylene;

each X is independently absent or 1 to 2 rings selected from arylene, cycloalkylene, heteroarylene, and heterocyclylene;

each X¹ is independently a heteroarylene or heterocyclylene ring;

each W is independently absent or 1 to 2 rings selected from arylene, cycloalkylene, heteroarylene, and heterocyclylene;

each W¹ is independently a heteroarylene or heterocyclylene ring;

each G is independently absent or a ring selected from arylene, cycloalkylene, heteroarylene, and heterocyclylene;

each G¹ and G² are independently heteroarylene or heterocyclylene ring;

each L¹ is independently selected from

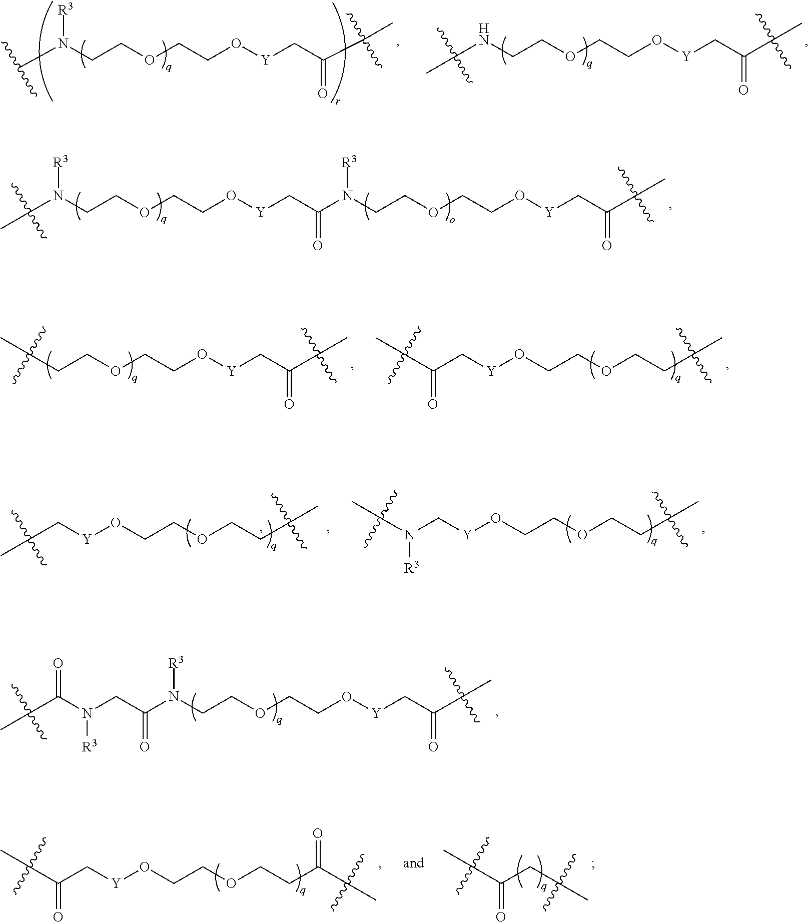

L² and L³ are independently absent or are independently selected from

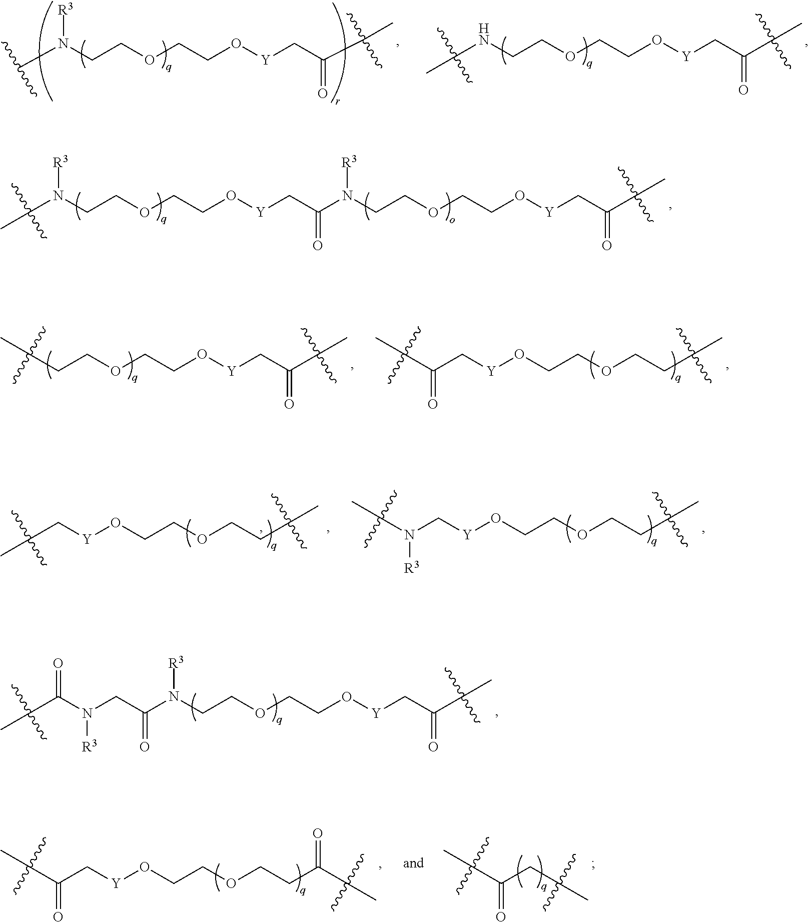

each B is independently selected from

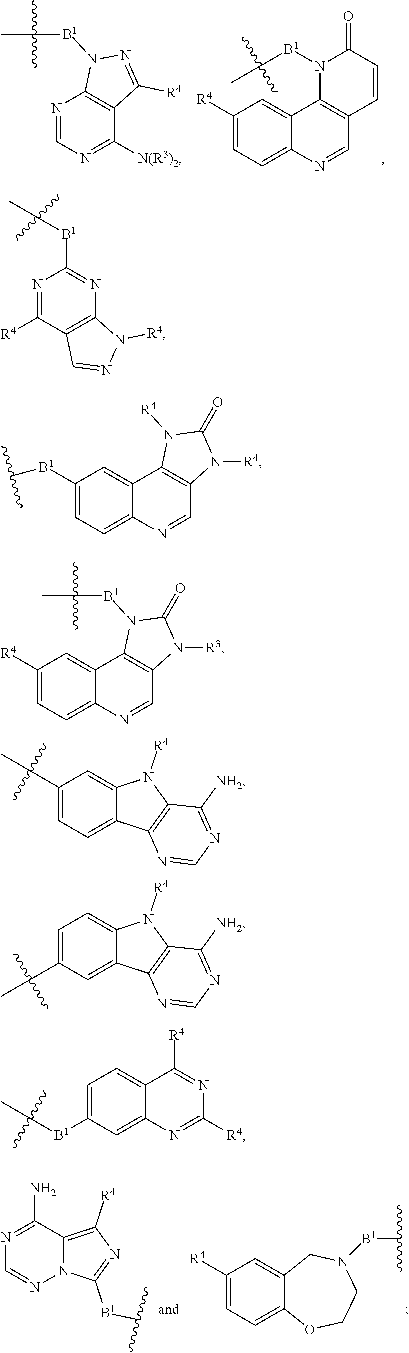

B¹ is selected from

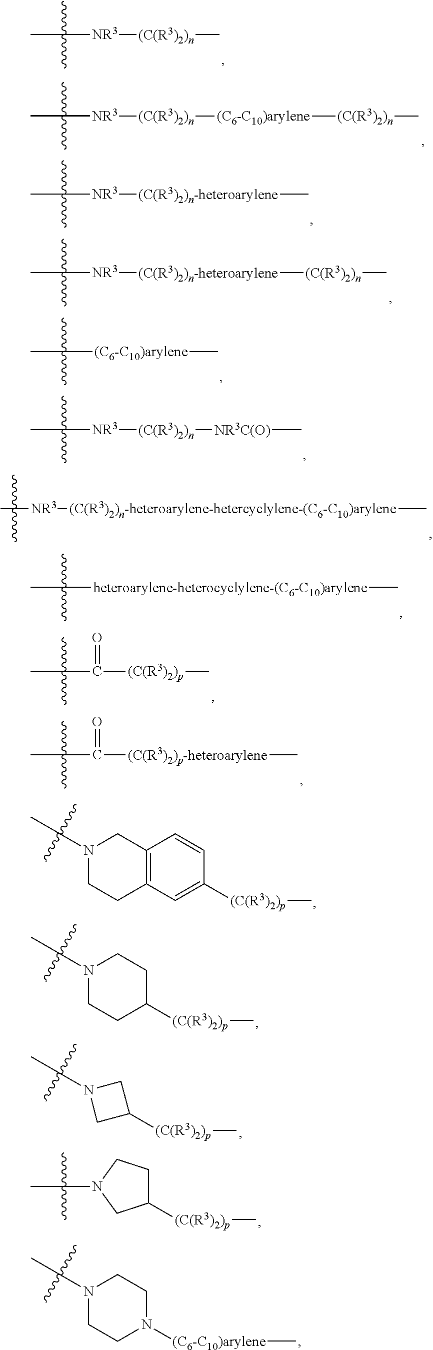

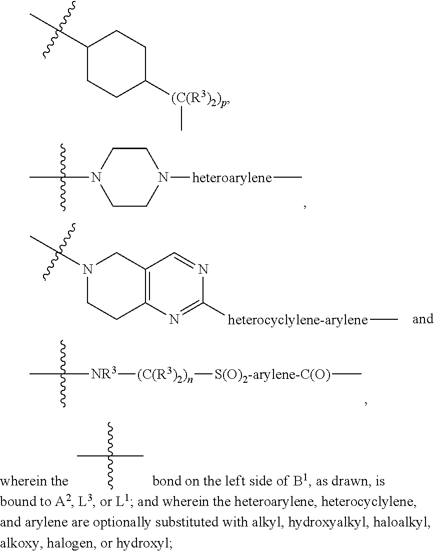

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each R⁵ is independently H, (C₁-C₆)alkyl, —C(O)OR³, or —N(R³)₂, wherein the alkyl of (C₁-C₆)alkyl is optionally substituted with —N(R³)₂ or —OR³;

each R⁶ is independently H, (C₁-C₆)alkyl, —C(O)OR³, or —N(R³)₂, wherein the alkyl of (C₁-C₆)alkyl is optionally substituted with —N(R³)₂ or —OR³;

each R⁷ is independently H, (C₁-C₆)alkyl, —C(O)OR³, or —N(R³)₂, wherein the alkyl of (C₁-C₆)alkyl is optionally substituted with —N(R³)₂ or —OR³;

each R⁸ is independently H, (C₁-C₆)alkyl, —C(O)OR³, or —N(R³)₂, wherein the alkyl of (C₁-C₆)alkyl is optionally substituted with —N(R³)₂ or —OR³;

each Y is independently C(R³)₂ or a bond;

each n is independently an integer from one to 12;

each o is independently an integer from zero to 30;

each p is independently an integer from zero to 12;

each q is independently an integer from zero to 30; and

each r is independently an integer from one to 6.

Embodiment II-2. The compound of Embodiment II-1, wherein R³² is ═O.

Embodiment II-3. The compound of Embodiment II-1, wherein R³² is —OR³.

Embodiment II-4. The compound of any one of Embodiments II-1 to II-3, or a pharmaceutically acceptable salt or tautomer thereof, wherein the compound is represented by the structure of Formula (I-40b):

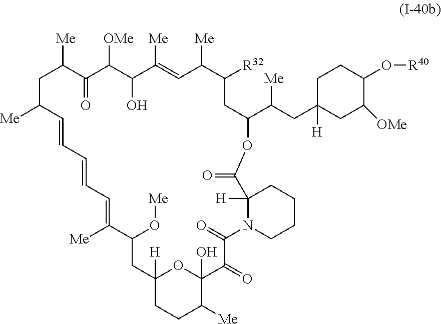

wherein R⁴⁰ is —C(═Z¹)—R^{40a}.

Embodiment II-5. The compound of Embodiment II-4, wherein Z¹ is O.

Embodiment II-6. The compound of Embodiment II-4, wherein Z¹ is S.

Embodiment II-7. The compound of any one of Embodiments II-4 to II-6, wherein R^{40a} is -A¹-L¹-A²-B, wherein A¹ and A² are absent.

Embodiment II-8. The compound of any one of Embodiments II-4 to II-6, wherein R^{40a} is -A¹-L¹-A²-B, wherein A² is absent.

Embodiment II-9. The compound of any one of Embodiments II-4 to II-6, wherein R^{40a} is -A¹-L¹-A²-B, wherein A¹ is absent.

Embodiment II-10. The compound of any one of Embodiments II-4 to II-6, wherein R^{40a} is -A¹-L¹-A²-B.

Embodiment II-11. The compound of any one of Embodiments II-4 to II-6, wherein R^{40a} is -A¹-A²-B.

Embodiment II-12. The compound of any one of Embodiments II-4 to II-6, wherein R^{40a} is -L²-A¹-L¹-A²-L³-B, wherein L² and A¹ are absent.

Embodiment II-13. The compound of any one of Embodiments II-4 to II-6, wherein R^{40a} is -L²-A¹-L¹-A²-L³-B, wherein L² is absent.

Embodiment II-14. The compound of any one of Embodiments II-4 to II-6, wherein R⁴⁰ is -L²-A¹-L¹-A²-L³-B, wherein L³ is absent.

Embodiment II-15. The compound of any one of Embodiments II-4 to II-6, wherein R^{40a} is —O—(C₁-C₆)alkyl or —O—(C₆-C₁₀)aryl; wherein the aryl of —O—(C₆-C₁₀)aryl is unsubstituted or substituted with 1-5 substituents selected from —NO₂ and halogen.

Embodiment II-16. The compound of any one of Embodiments II-1 to II-3, or a pharmaceutically acceptable salt or tautomer thereof, wherein the compounds are represented by the structure of Formula (I-28b):

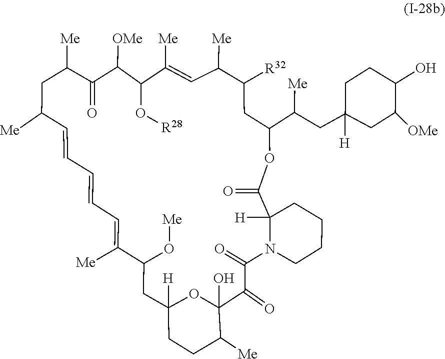

wherein R²⁸ is —C(═Z¹)—R^{28a}.

Embodiment II-17. The compound of Embodiment II-16, wherein Z¹ is O.

Embodiment II-18. The compound of Embodiment II-16, wherein Z¹ is S.

Embodiment II-19. The compound of any one of Embodiments II-16 to II-18, wherein R^{28a} is -A¹-L¹-A²-B, wherein A¹ and A² are absent.

Embodiment II-20. The compound of any one of Embodiments II-16 to II-18, wherein R^{28a} is -A¹-L¹-A²-B, wherein A² is absent.

Embodiment II-21. The compound of any one of Embodiments II-16 to II-18, wherein R^{28a} is -A¹-L¹-A²-B, wherein A¹ is absent.

Embodiment II-22. The compound of any one of Embodiments II-16 to II-18, wherein R^{28a} is -A¹-L¹-A²-B.

Embodiment II-23. The compound of any one of Embodiments II-16 to II-18, wherein R^{28a} is -A¹-A²-B.

Embodiment II-24. The compound of any one of Embodiments II-16 to II-18, wherein R^{28a} is -L²-A¹-L¹-A²-L¹-B, wherein L² and A¹ are absent.

Embodiment II-25. The compound of any one of Embodiments II-16 to II-18, wherein R^{28a} is -L²-A¹-L¹-A²-L³-B, wherein L² is absent.

Embodiment II-26. The compound of any one of Embodiments II-16 to II-18, wherein R^{28a} is -L²-A¹-L¹-A²-L¹-B, wherein L³ is absent.

Embodiment II-27. The compound of any one of Embodiments II-16 to II-18, wherein R^{28a} is —O—(C₁-C₆)alkyl or —O—(C₆-C₁₀)aryl; wherein the aryl of —O—(C₆-C₁₀)aryl is unsubstituted or substituted with 1-5 substituents selected from —NO₂ and halogen.

Embodiment II-28. The compound of Embodiment II-1, or a pharmaceutically acceptable salt or tautomer thereof, wherein the compound is represented by the structure of Formula (I-32b):

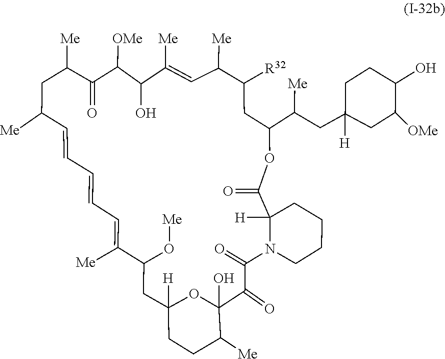

wherein R³² is —O—C(═Z¹)—R^{32a}.

Embodiment II-29. The compound of Embodiment II-28, wherein Z¹ is O.

Embodiment II-30. The compound of Embodiment II-28, wherein Z¹ is S.

Embodiment II-31. The compound of any one of Embodiments II-28 to II-30, wherein R^{32a} is -A¹-L¹-A²-B, wherein A¹ and A² are absent.

Embodiment II-32. The compound of any one of Embodiments II-28 to II-30, wherein R^{32a} is -A¹-L¹-A²-B, wherein A² is absent.

Embodiment II-33. The compound of any one of Embodiments II-28 to II-30, wherein R^{32a} is -A¹-L¹-A²-B, wherein A¹ is absent.

Embodiment II-34. The compound of any one of Embodiments II-28 to II-30, wherein R^{32a} is -A¹-L¹-A²-B.

Embodiment II-35. The compound of any one of Embodiments II-28 to II-30, wherein R^{32a} is -A¹-A²-B.

Embodiment II-36. The compound of any one of Embodiments II-28 to II-30, wherein R^{32a} is -L²-A¹-L¹-A²-L³-B, wherein L² and A¹ are absent.

Embodiment II-37. The compound of any one of Embodiments II-28 to II-30, wherein R^{32a} is -²-A¹-L¹-A²-L³-B, wherein L² is absent.

Embodiment II-38. The compound of any one of Embodiments II-28 to II-30, wherein R^{32a} is -L²-A¹-L¹-A²-L³-B, wherein L³ is absent.

Embodiment II-39. The compound of any one of Embodiments II-28 to II-30, wherein R^{32a} is —O—(C₁-C₆)alkyl or —O—(C₆-C₁₀)aryl; wherein the aryl of —O—(C₆-C₁₀)aryl is unsubstituted or substituted with 1-5 substituents selected from —NO₂ and halogen.

Embodiment II-40. The compound of any one of Embodiments II-1 to II-10, II-12 to II-22, II-24 to II-35, and II-36 to II-39, wherein L¹ is

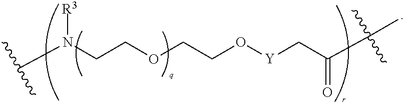

Embodiment II-41. The compound of any one Embodiments II-1 to II-10 II-12 to II-22, II-24 to II-35, and II-36 to II-39, wherein L¹ is

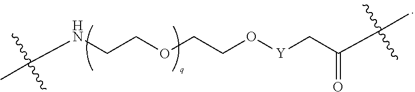

Embodiment II-42. The compound of any one of Embodiments II-1 to II-10, II-12 to II-22, 24-35, and 36-39, wherein L¹ is

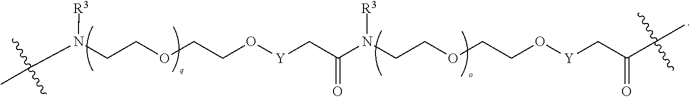

Embodiment II-43. The compound of any one of Embodiments II-1 to II-10, II-12 to II-22, II-24 to II-35, and II-36 to II-39, wherein L¹ is

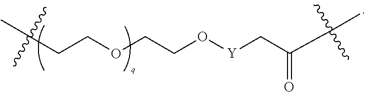

Embodiment II-44. The compound of any one of Embodiments II-1 to II-10, II-12 to II-22, II-24 to II-35, and II-36 to II-39, wherein L¹ is

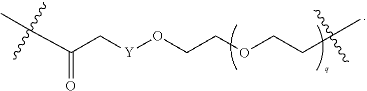

Embodiment II-45. The compound of any one of Embodiments II-1 to II-10, II-12 II-22, II-24 to II-35, and II-36 to II-39, wherein L¹ is

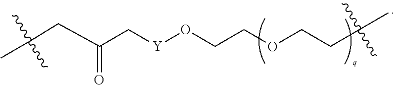

Embodiment II-46. The compound of any one of Embodiments II-1 to II-6, II-12 to II-18, II-24 to II-30, and II-36 to II-45, wherein L² is

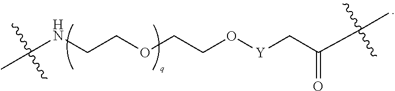

Embodiment II-47. The compound of any one of Embodiments II-1 to II-6, II-12 to II-18, II-24 to II-30, and II-36 to II-45, wherein L³ is

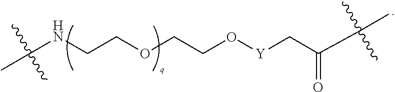

Embodiment II-48. The compound of any one of Embodiments II-1 to II-7, II-9, II-12, II-16 to II-19, H-21, II-24, II-28 to II-31, II-33, II-36, and II-39 to II-45, wherein A¹ is absent.

Embodiment II-49. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

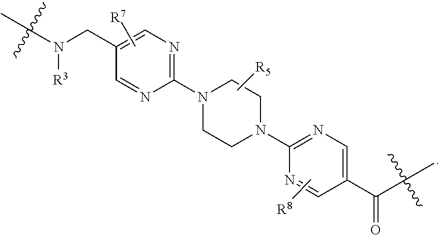

Embodiment II-50. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

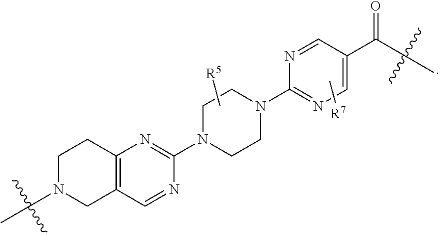

Embodiment II-51. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

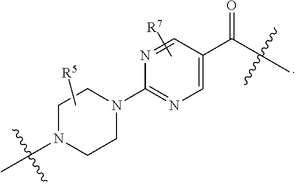

Embodiment II-52. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

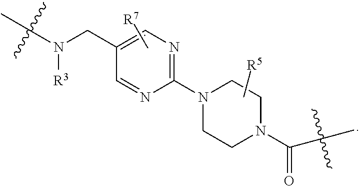

Embodiment II-53. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

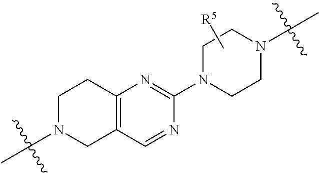

Embodiment II-54. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

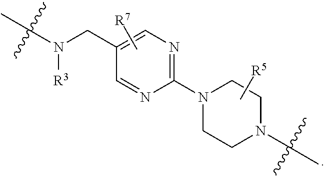

Embodiment II-55. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

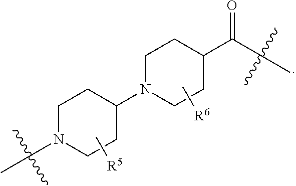

Embodiment II-56. The compound of any one of Embodiments II-1 to II-6, II-8, II-10 to II-11, II-13 to II-18, II-20, II-22 to I-23, II-25 to II-30, II-32, II-34 to II-35, and II-37 to II-45, wherein A¹ is

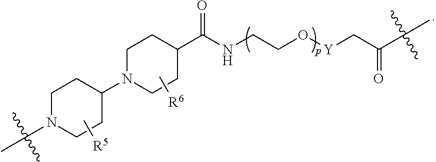

Embodiment II-57. The compound of any one of Embodiments II-1 to II-8, II-15 to II-20, II-27 to II-32, and II-39 to II-45, wherein A² is absent.

Embodiment II-58. The compound of any one of Embodiments II-1 to II-6, II-9 to II-18, II-21 to II-30, and II-33 to II-45, wherein A² is

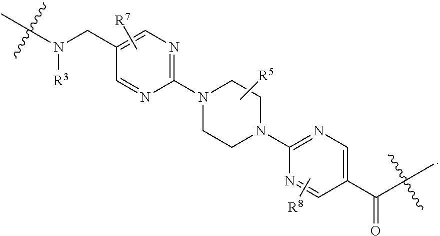

Embodiment II-59. The compound of any one of Embodiments II-1 to II-6, II-9 to II-18, II-21 to II-30, and II-33 to II-45, wherein A² is

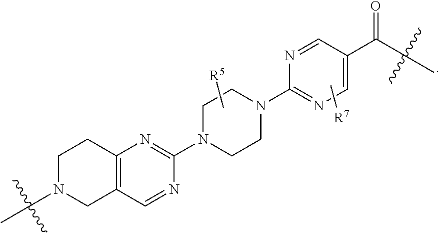

Embodiment II-60. The compound of any one of Embodiments II-1 to II-6, II-9 to II-18, II-21 to II-30, and II-33 to II-45, wherein A² is

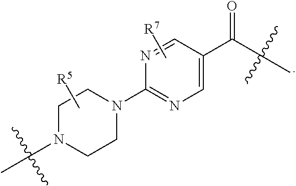

Embodiment II-61. The compound of any one of Embodiments II-1 to II-6, II-9 to II-18, II-21 to II-30, and II-33 to II-45, wherein A² is

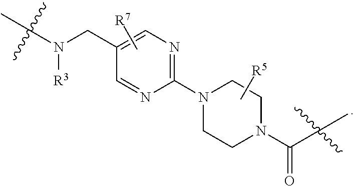

Embodiment II-62. The compound of any one of Embodiments II-1 to II-6, II-9 to II-18, II-21 to II-30, and II-33 to II-45, wherein A² is

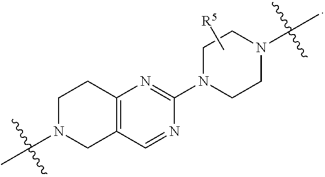

Embodiment II-63. The compound of any one of Embodiments II-1 to II-6, II-9 to II-18, II-21 to II-30, and II-33 to II-45, wherein A² is

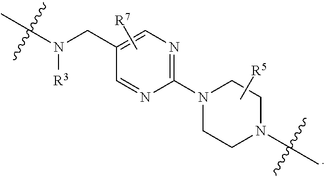

Embodiment II-64. The compound of any one of Embodiments II-1 to II-6, II-9 to II-18, II-21 to II-30, and II-33 to II-45, wherein A² is

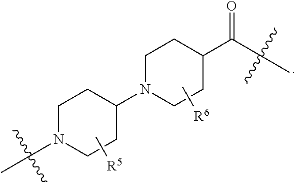

Embodiment II-65. The compound of any one of Embodiments II-1 to II-6, II-9 to II-18, II-21 to II-30, and II-33 to II-45, wherein A² is

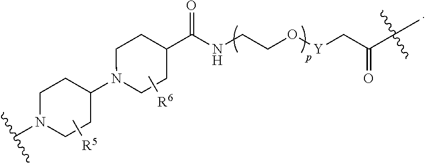

Embodiment II-66. The compound of any one of Embodiments II-1 to II-65, wherein B is

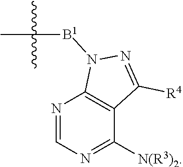

Embodiment II-67. The compound of any one of Embodiments II-1 to II-65, wherein B is

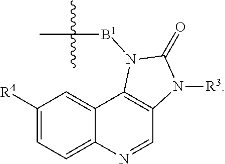

Embodiment II-68. The compound of any one of Embodiments II-1 to II-65, wherein B¹ is

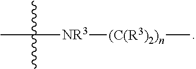

Embodiment II-69. The compound of any one of Embodiments II-1 to II-65, wherein B¹ is

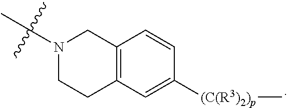

Embodiment II-70. The compound of any one of Embodiments II-1 to II-69, wherein R⁴ is 5-12 membered heteroaryl, optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl.

Embodiment II-71. The compound of any one of Embodiments II-1 to II-70, or a pharmaceutically acceptable salt or tautomer thereof, wherein compound has the following formula:

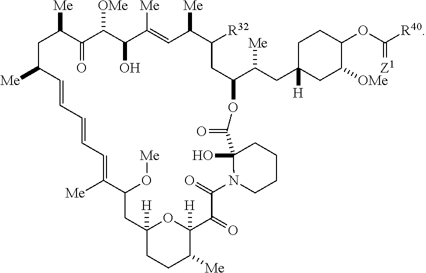

Embodiment II-72. A compound of selected from the group consisting of:

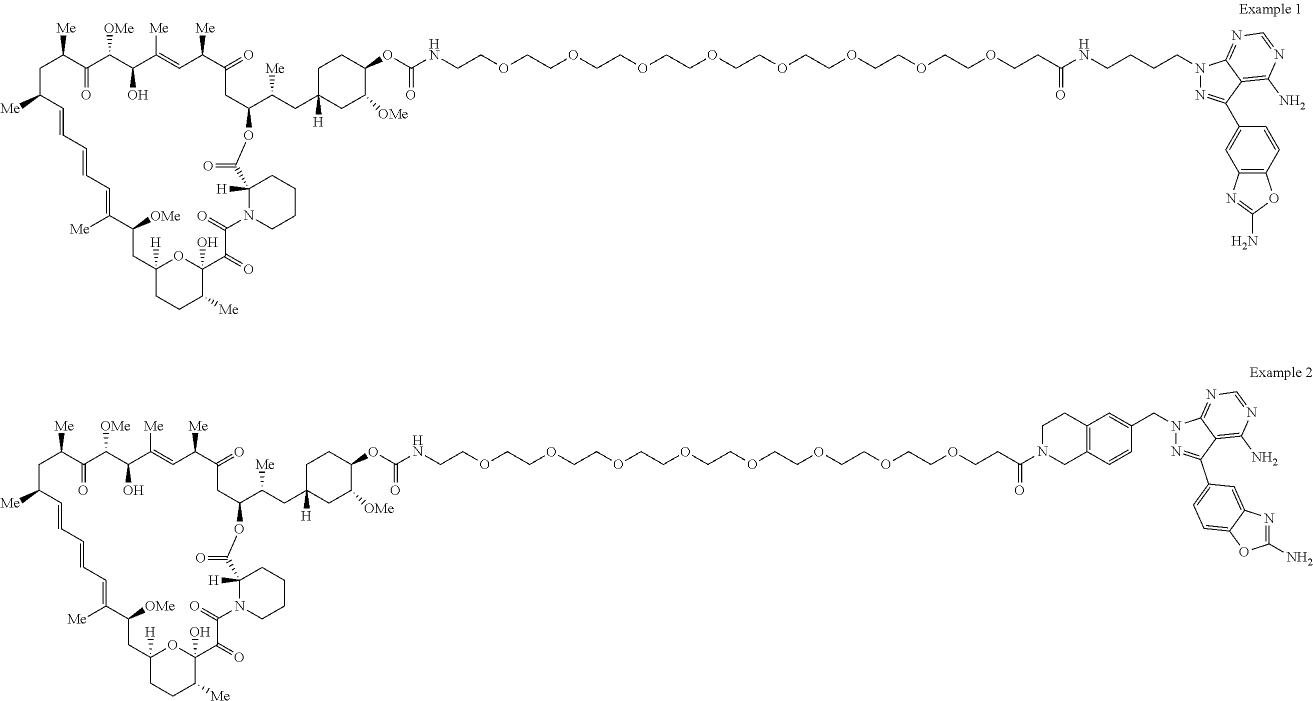

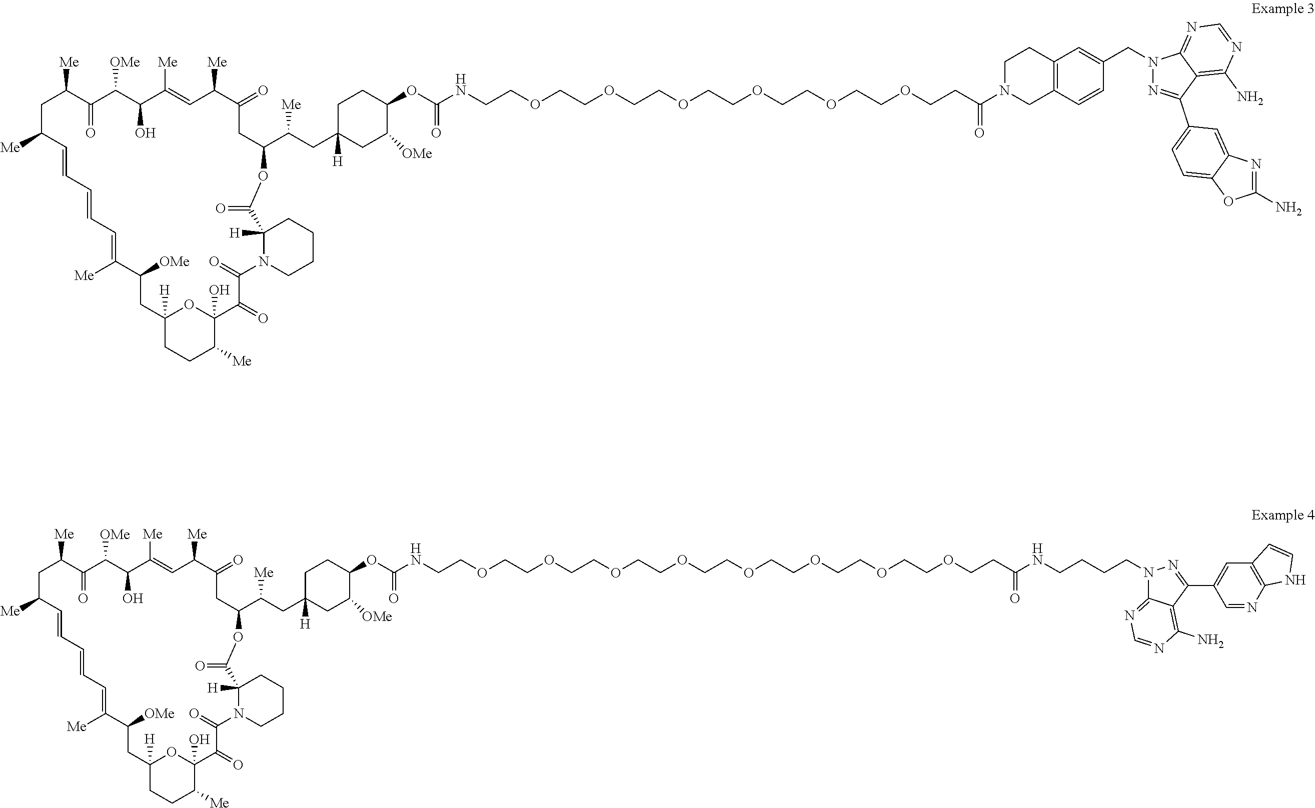

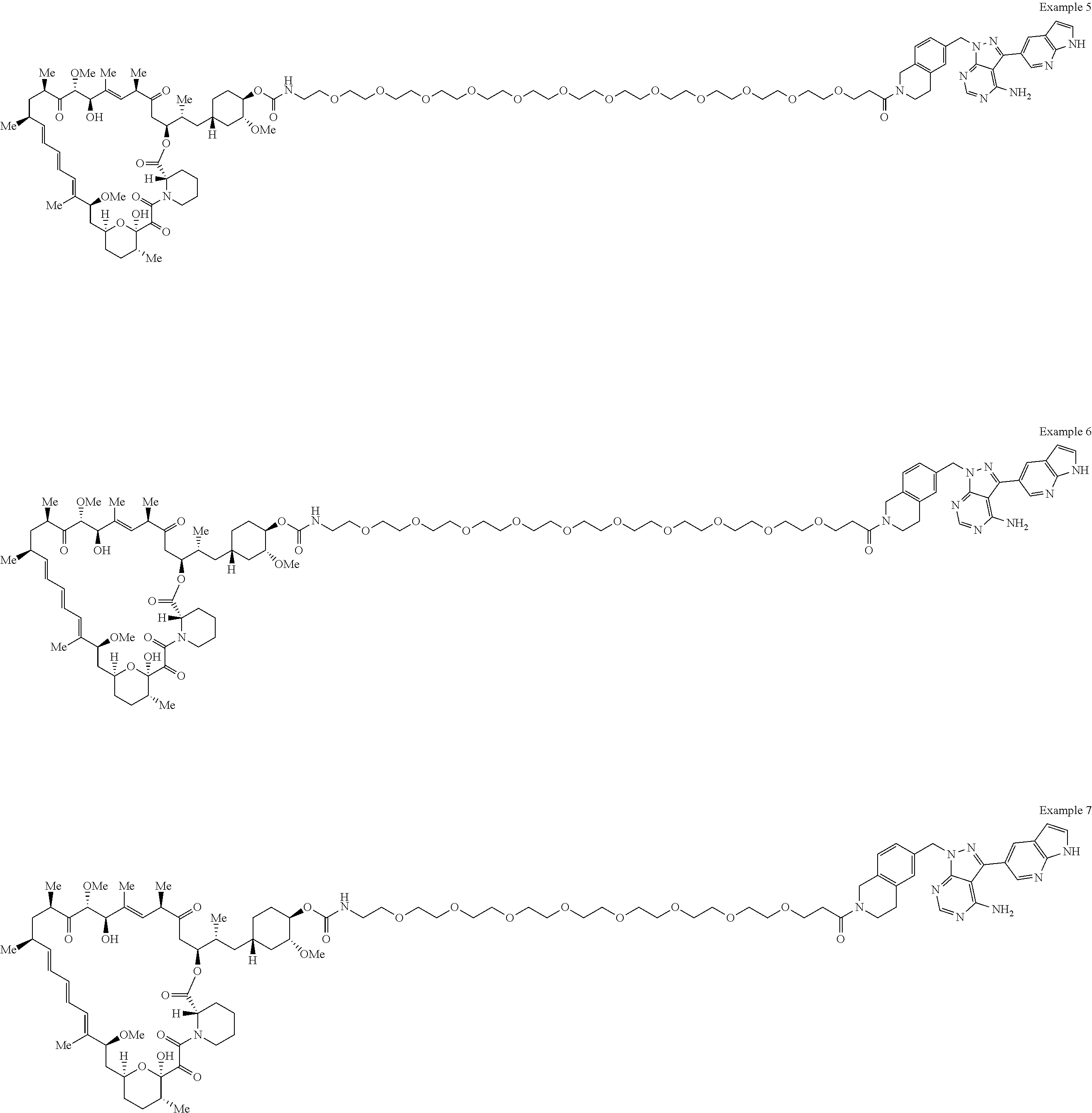

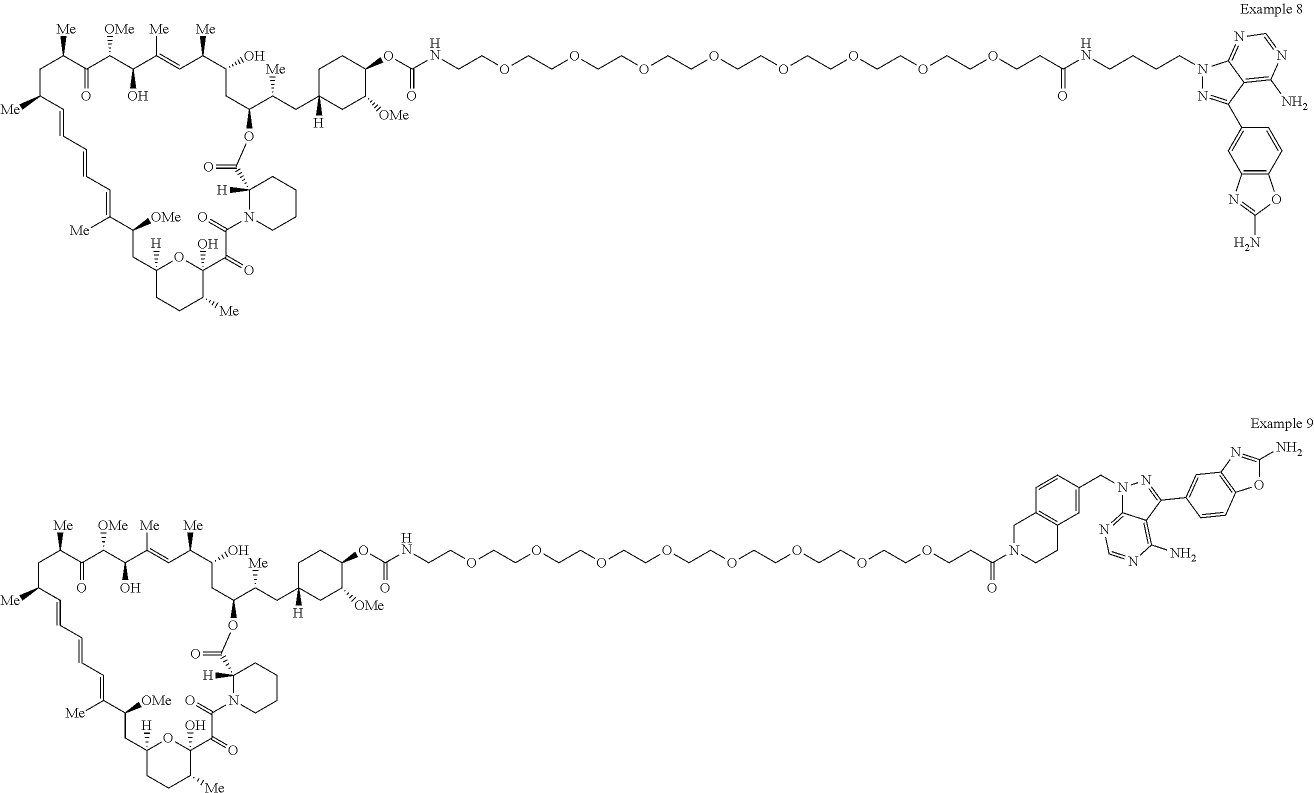

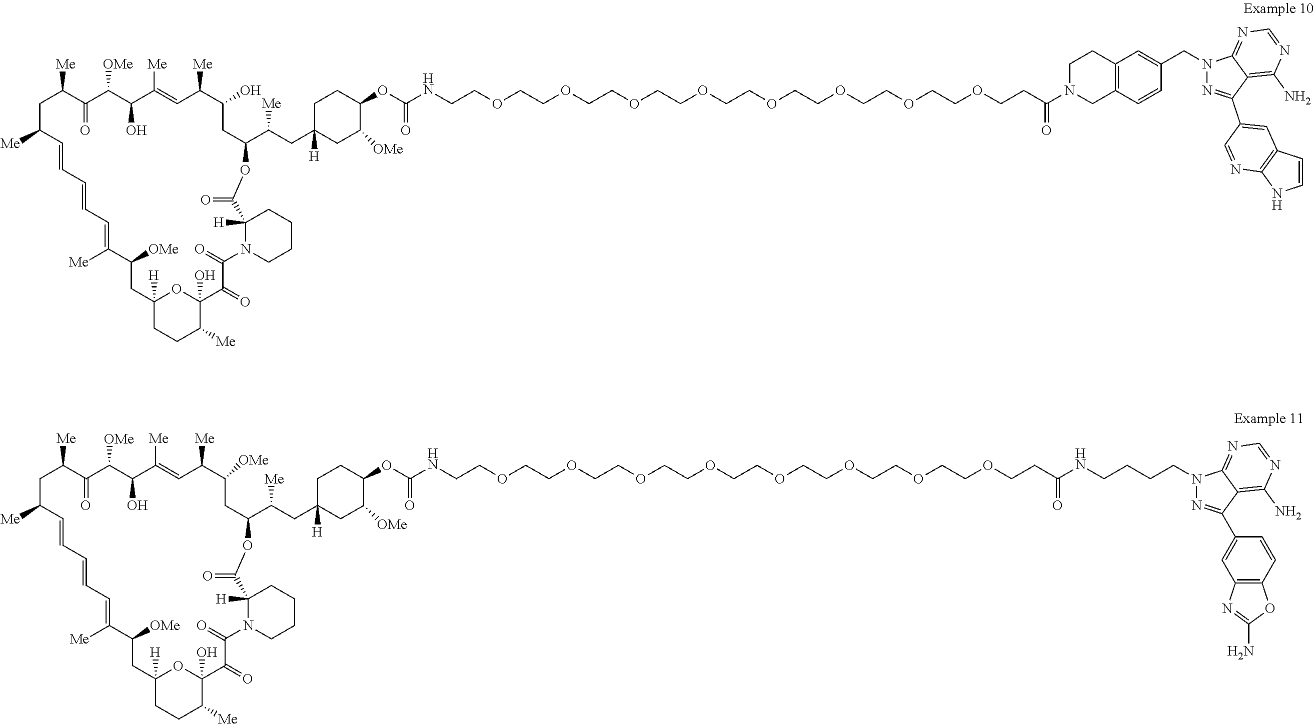

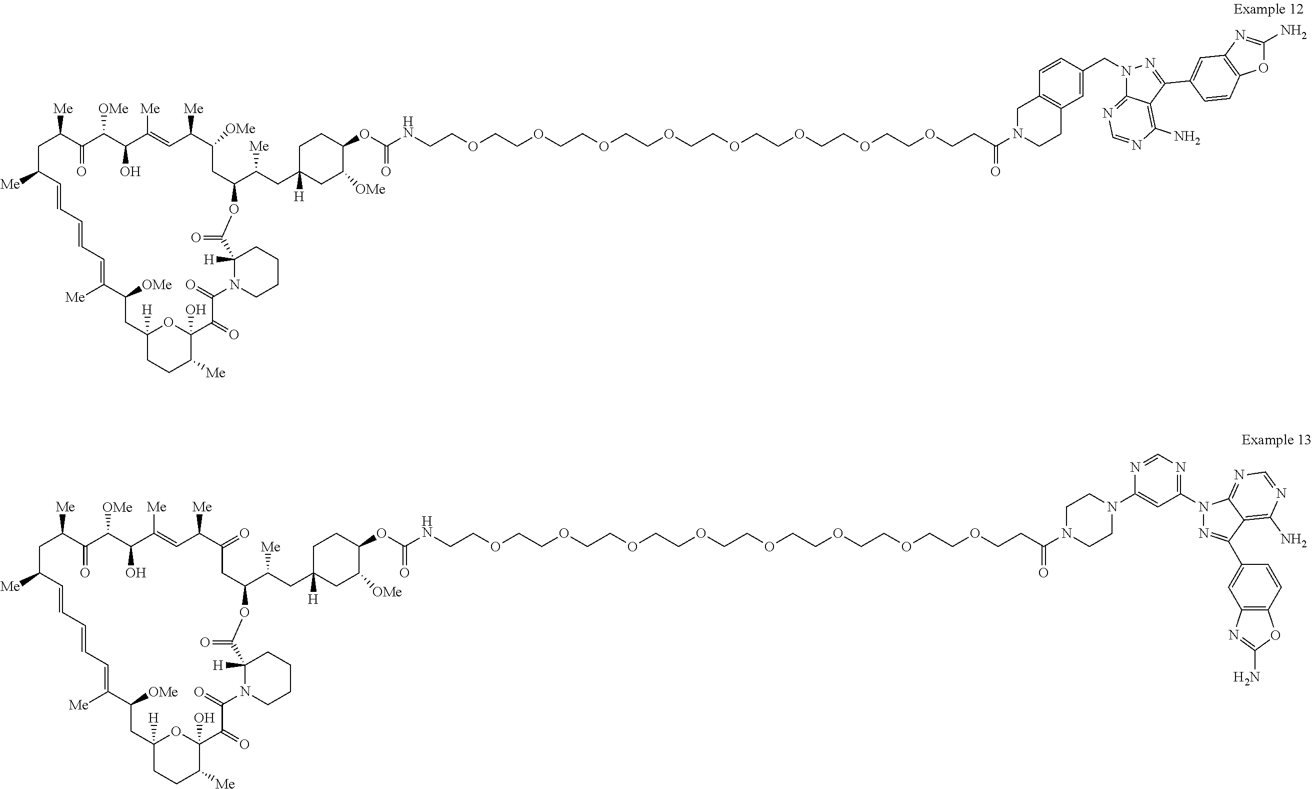

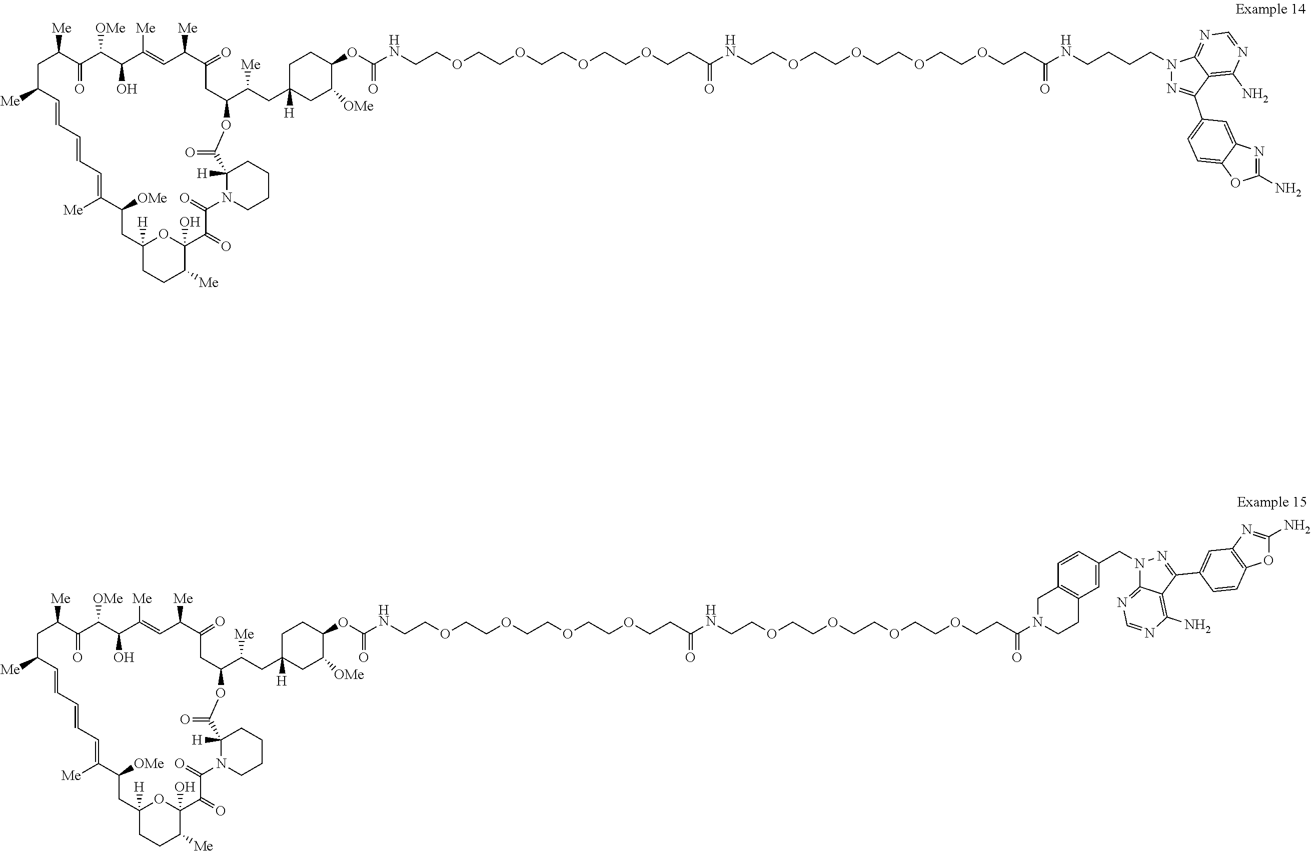

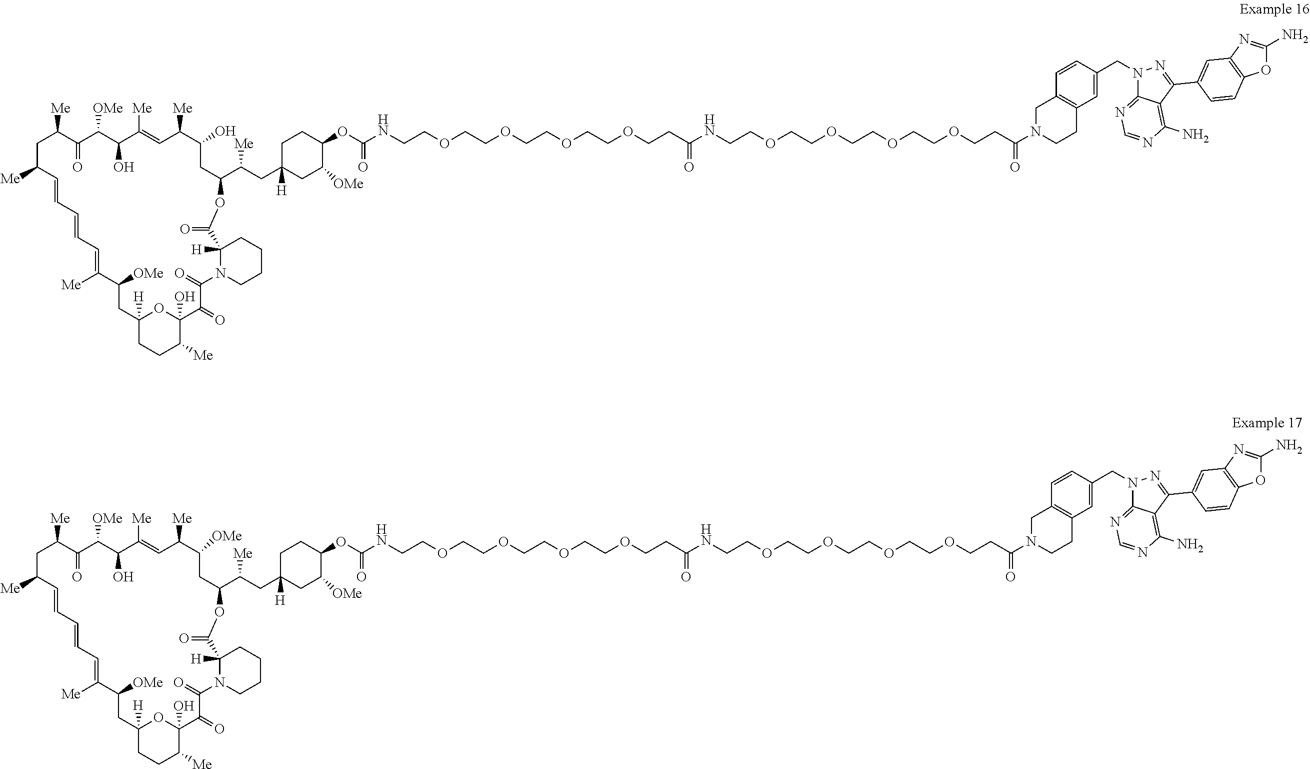

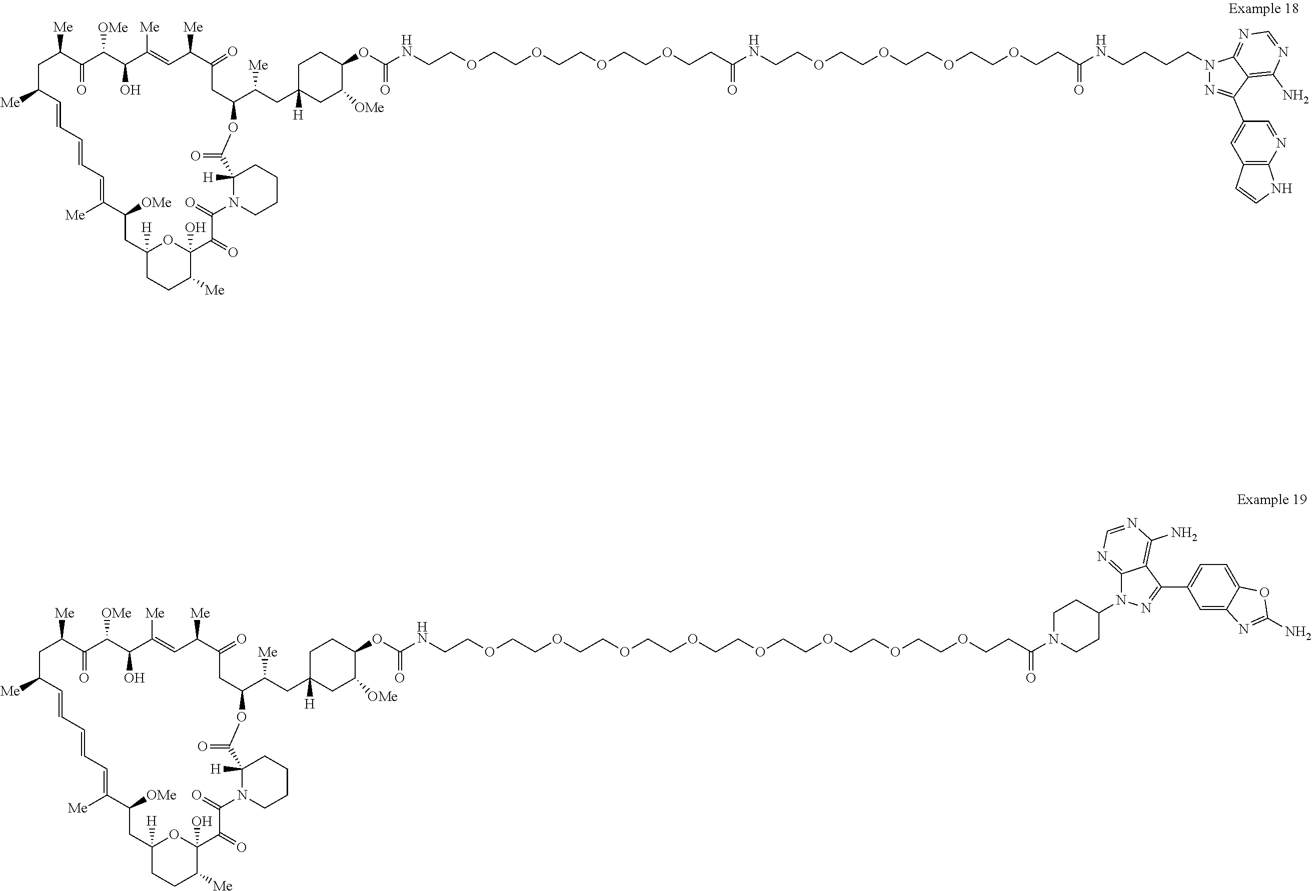

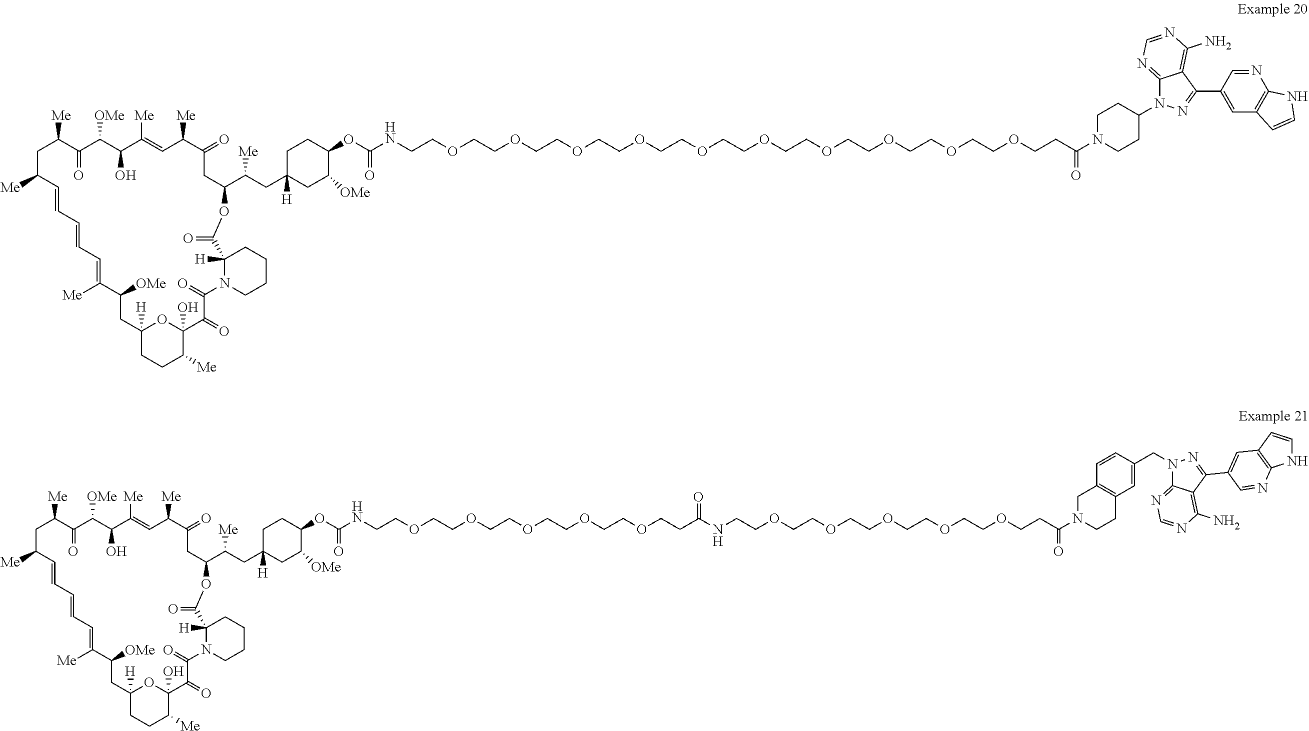

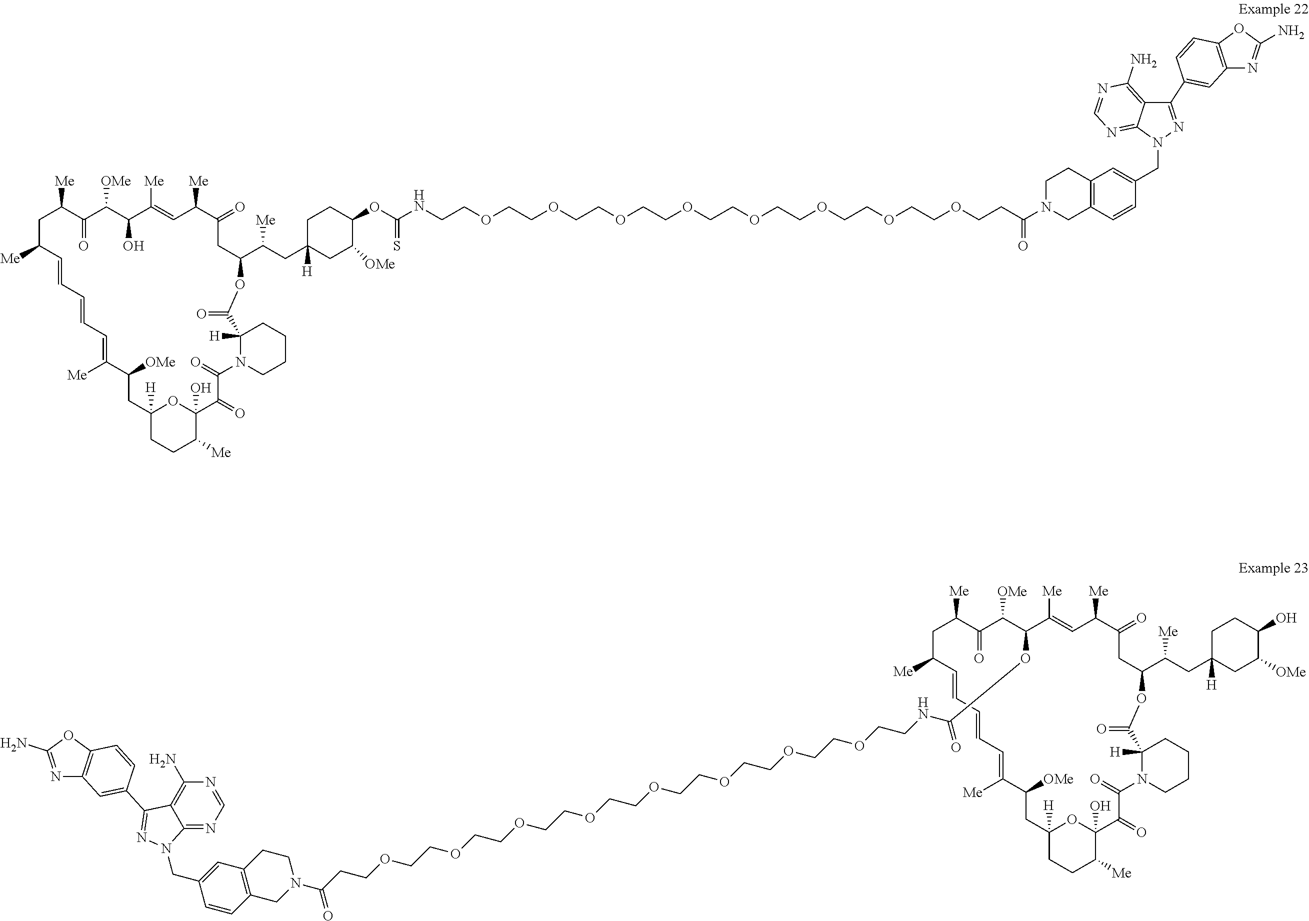

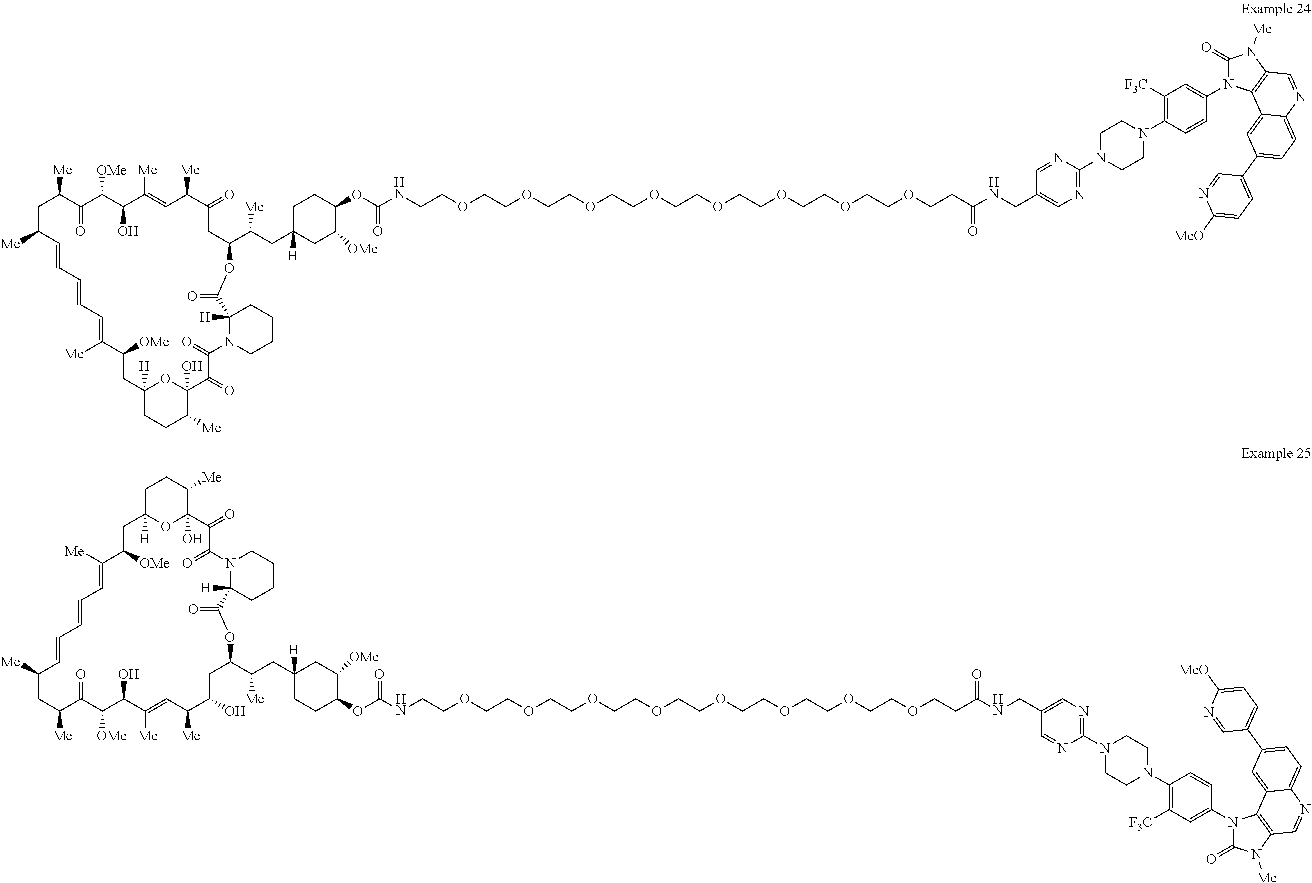

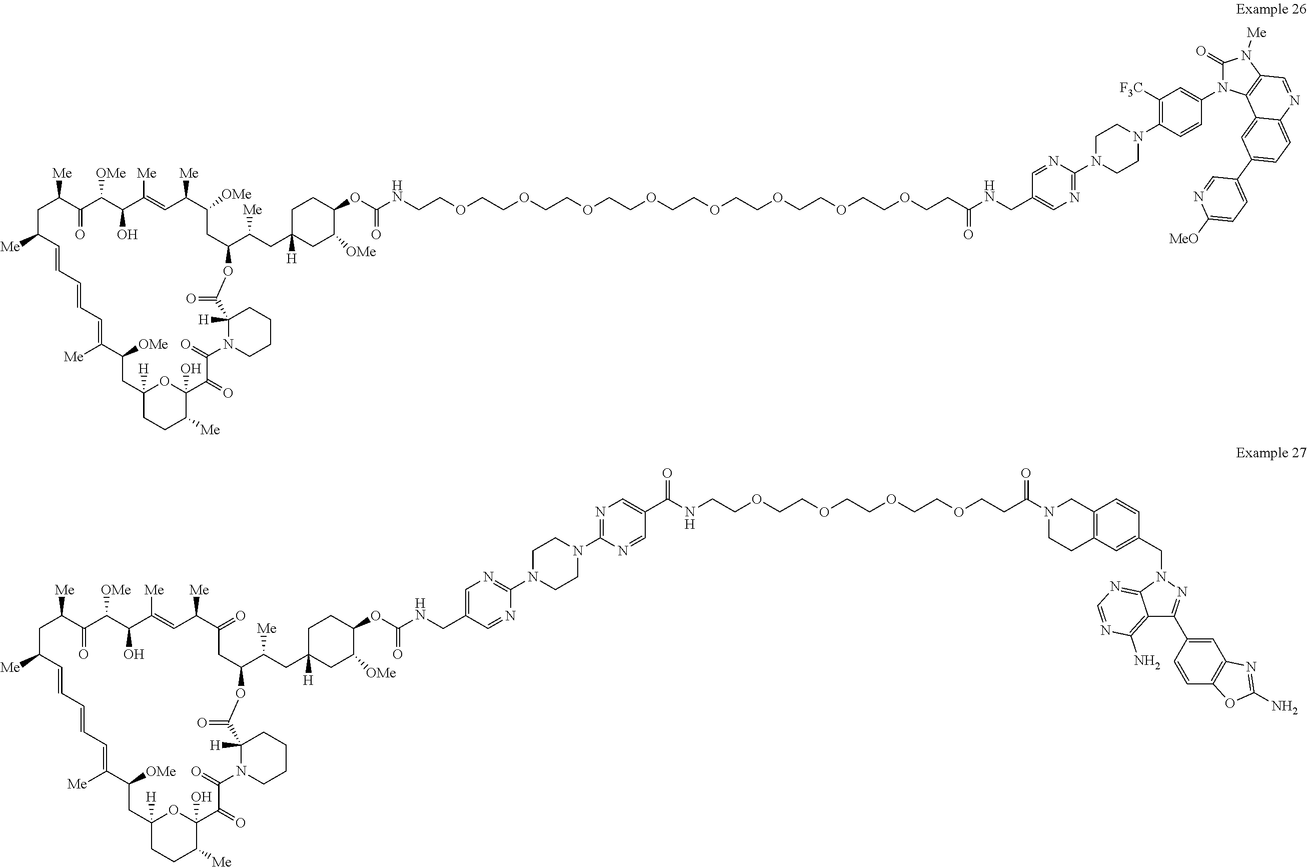

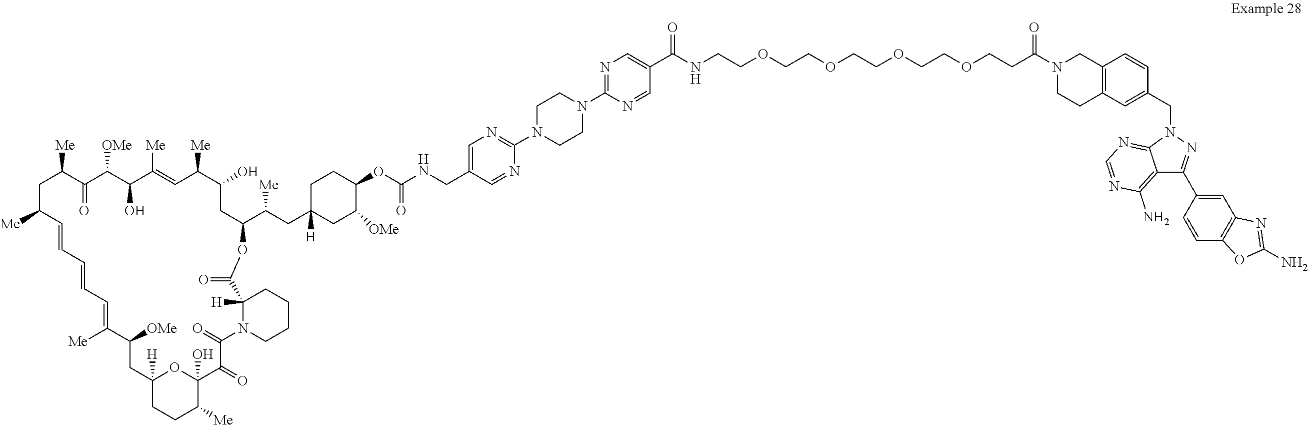 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

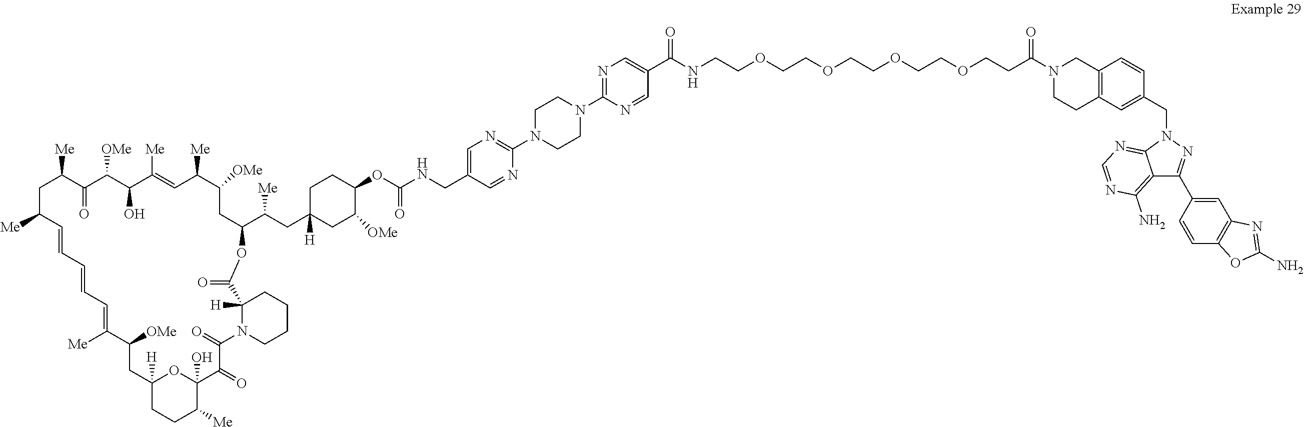 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

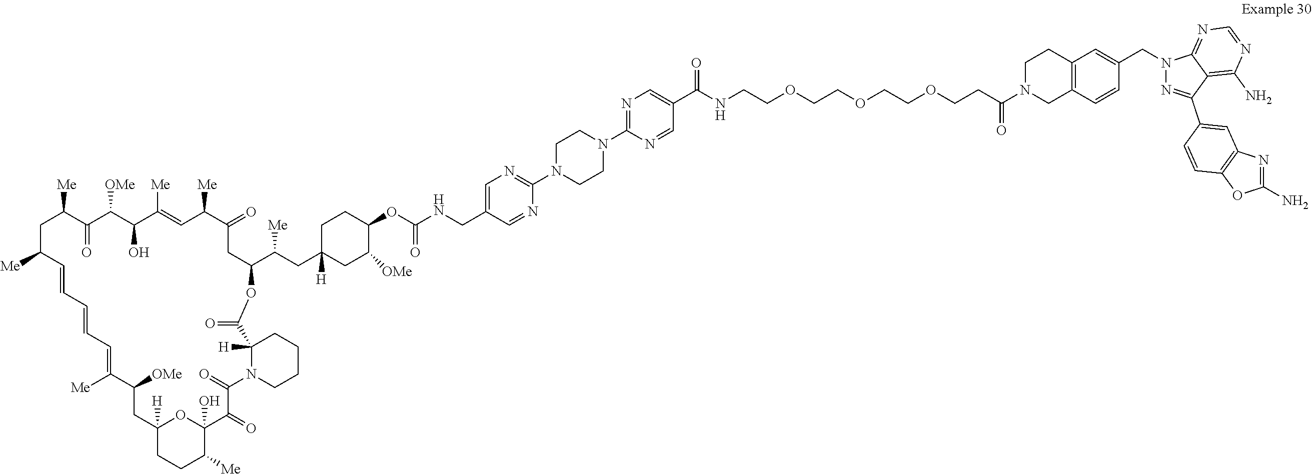 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

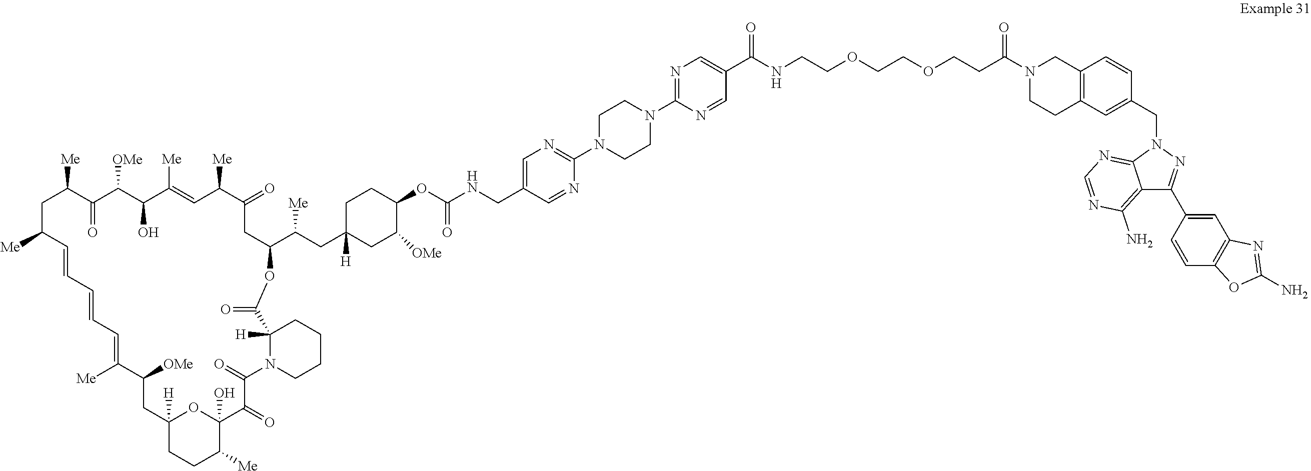 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

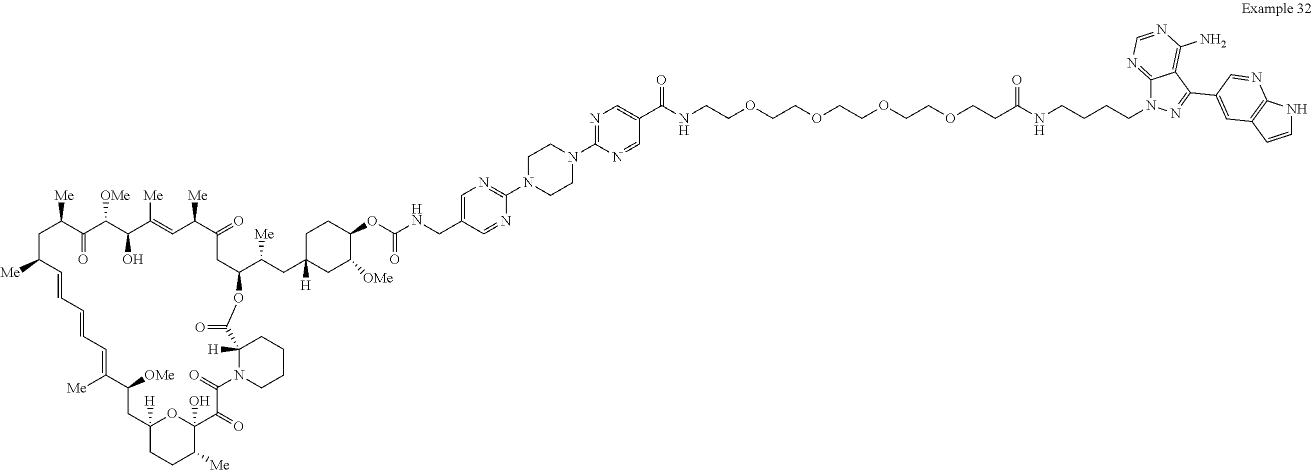 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

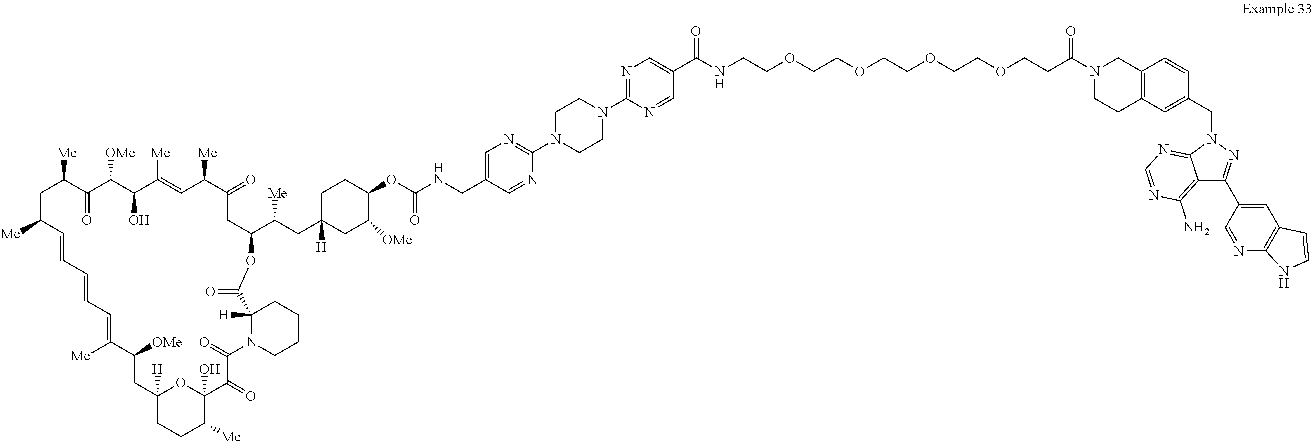 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

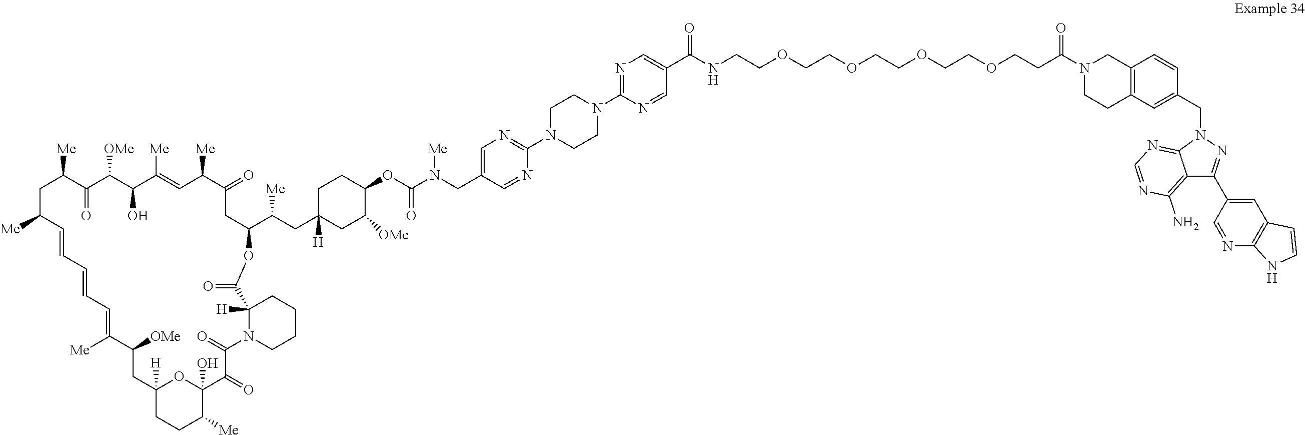 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

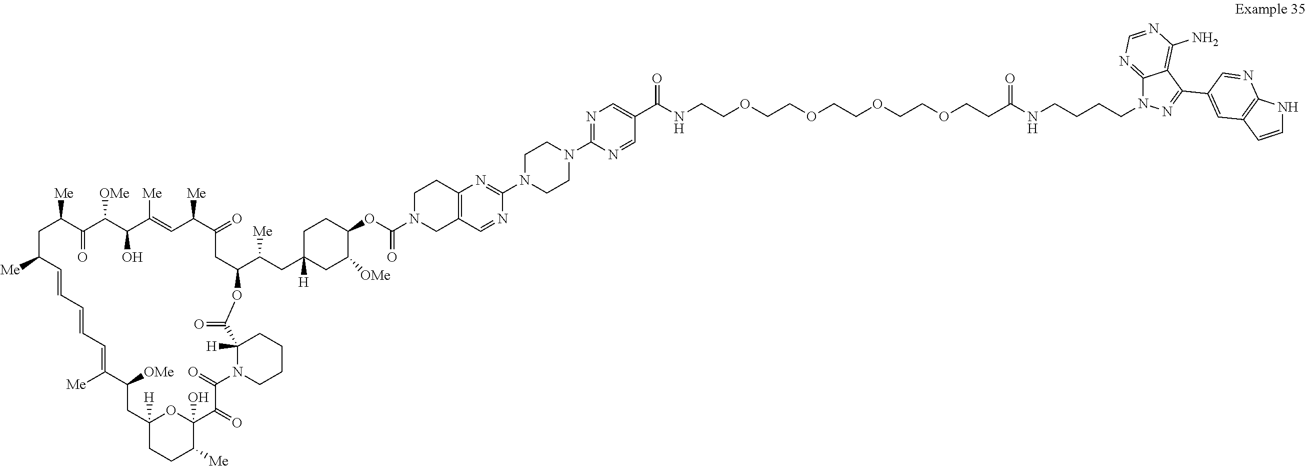 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

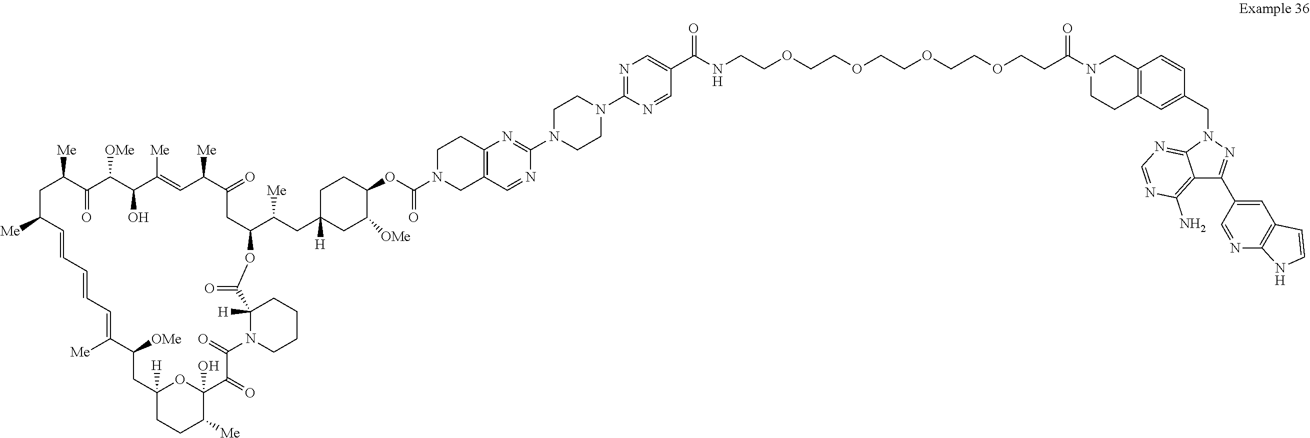 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

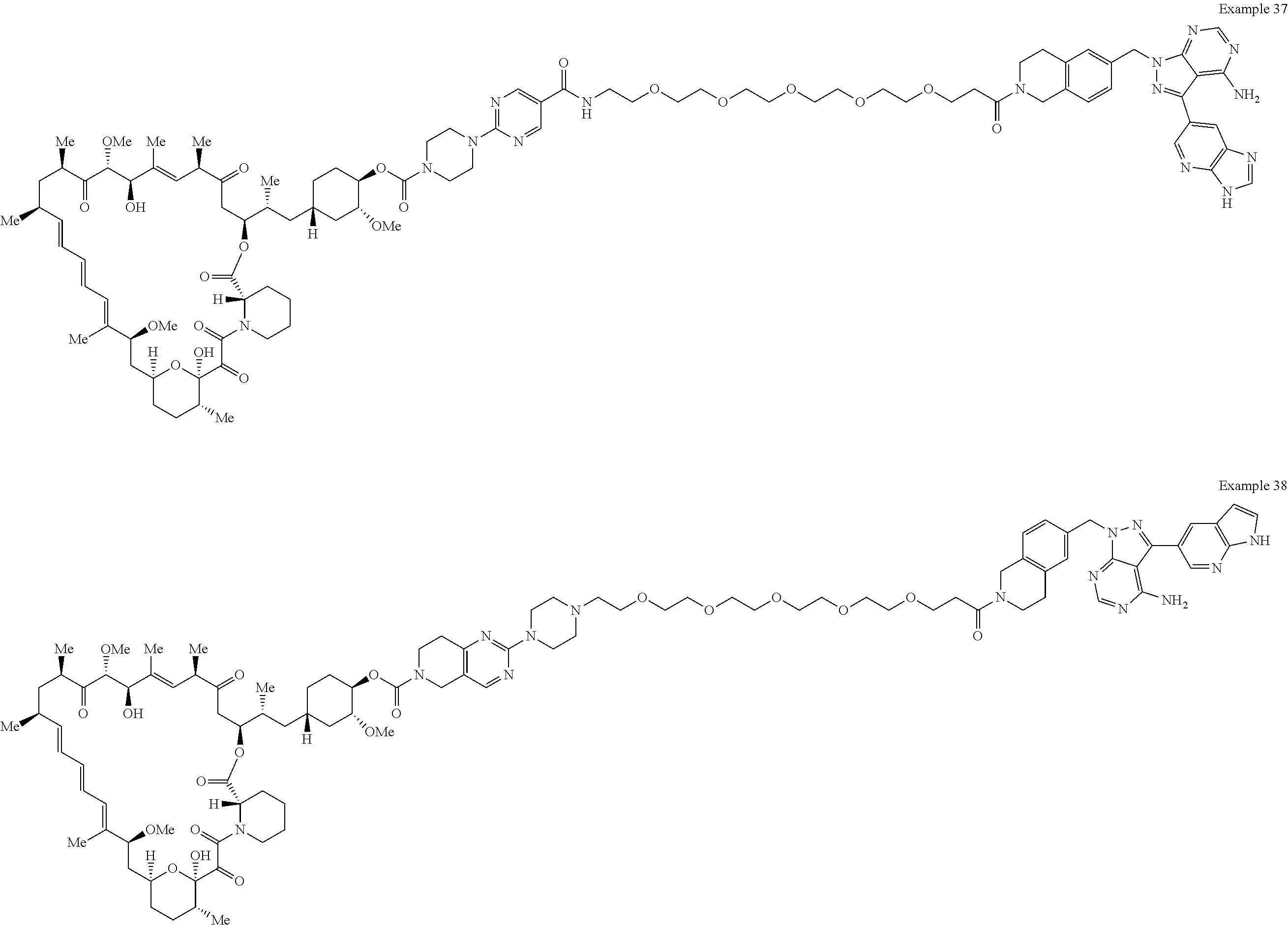

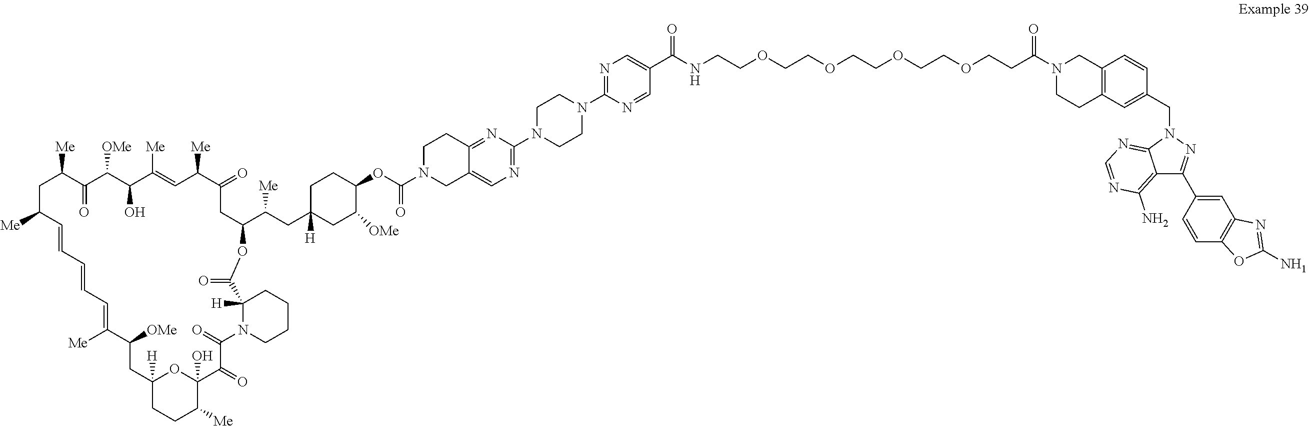 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

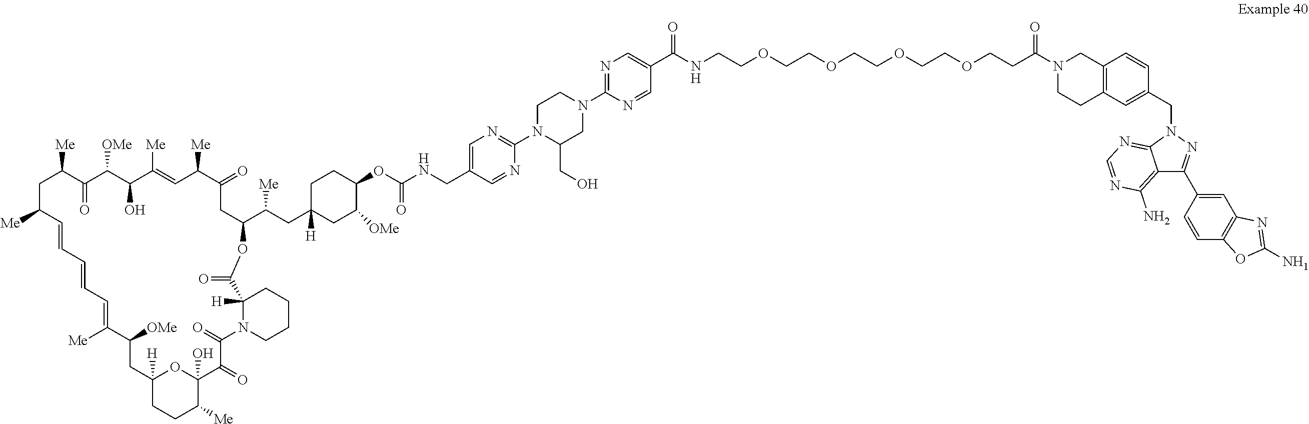 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

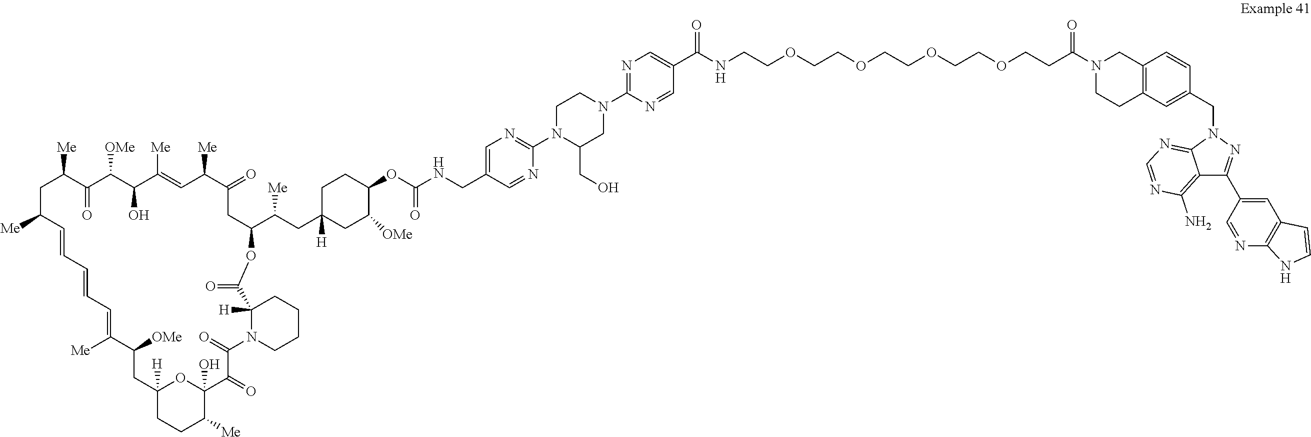 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

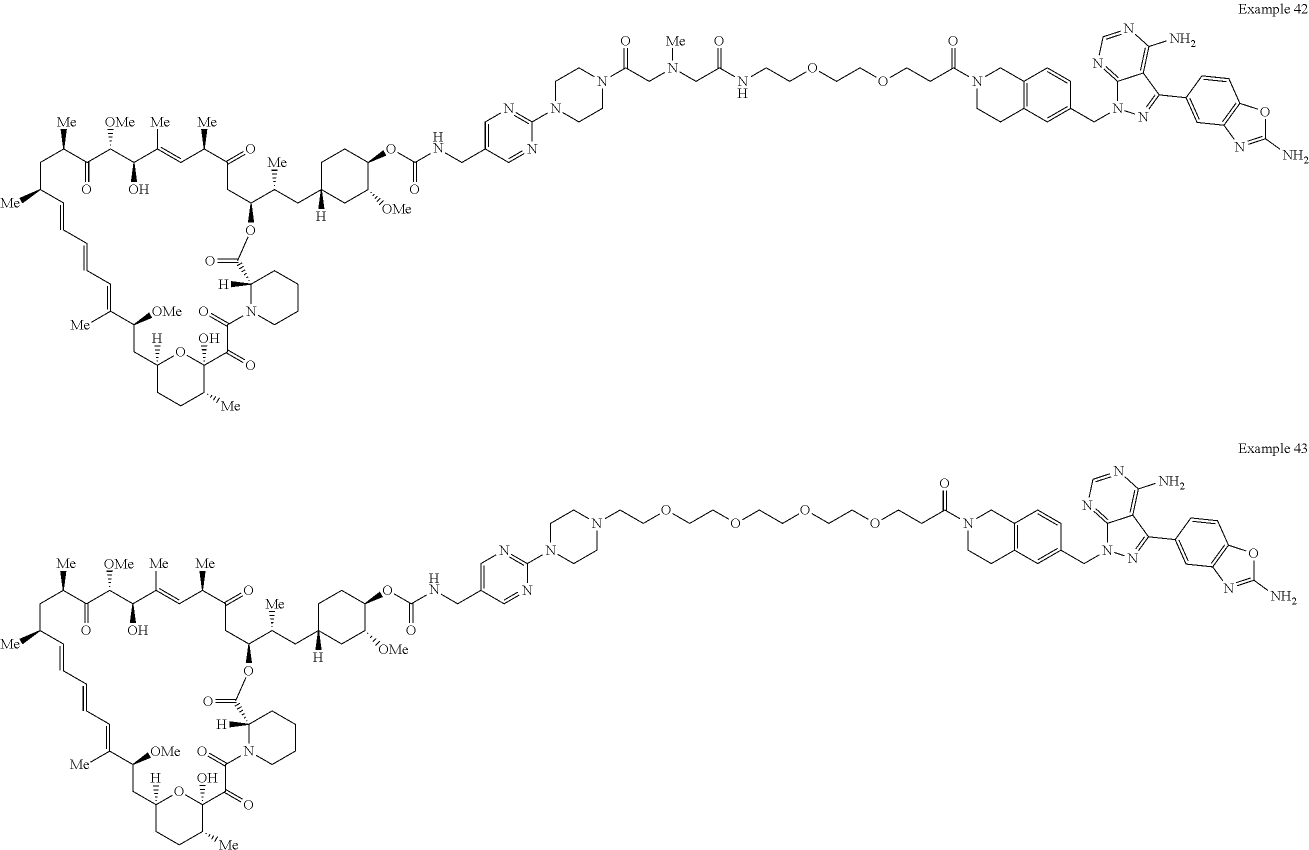

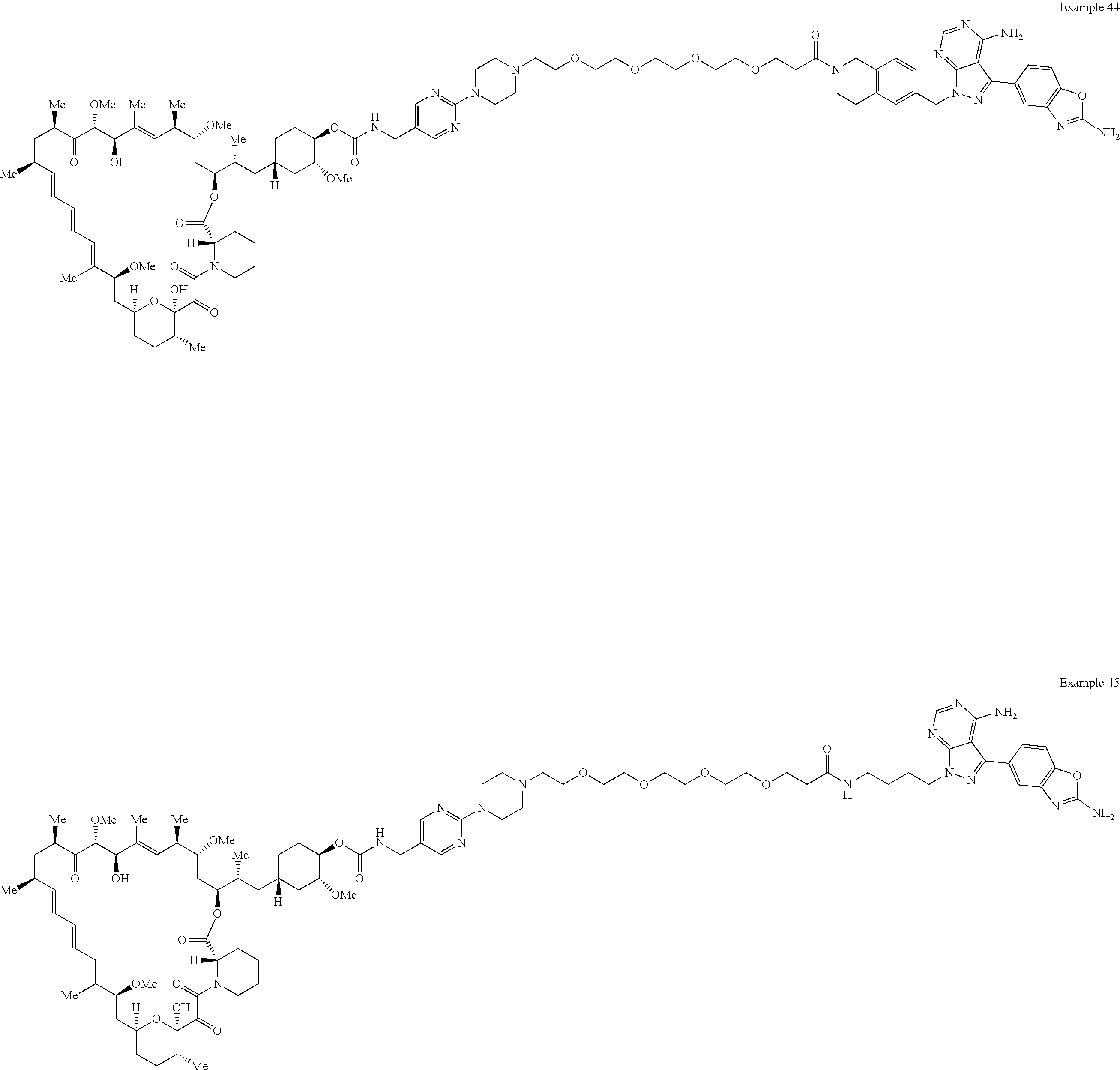

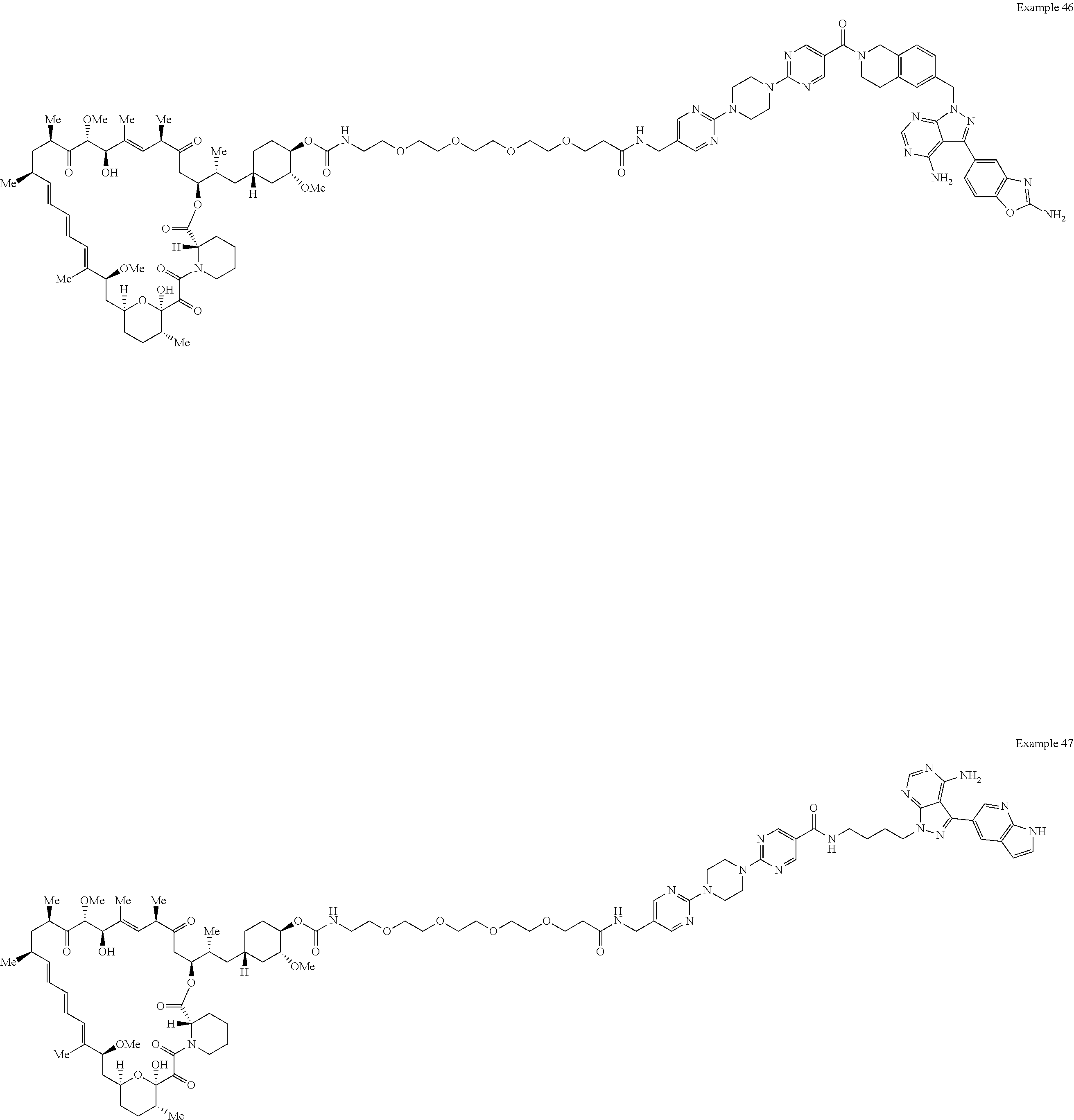

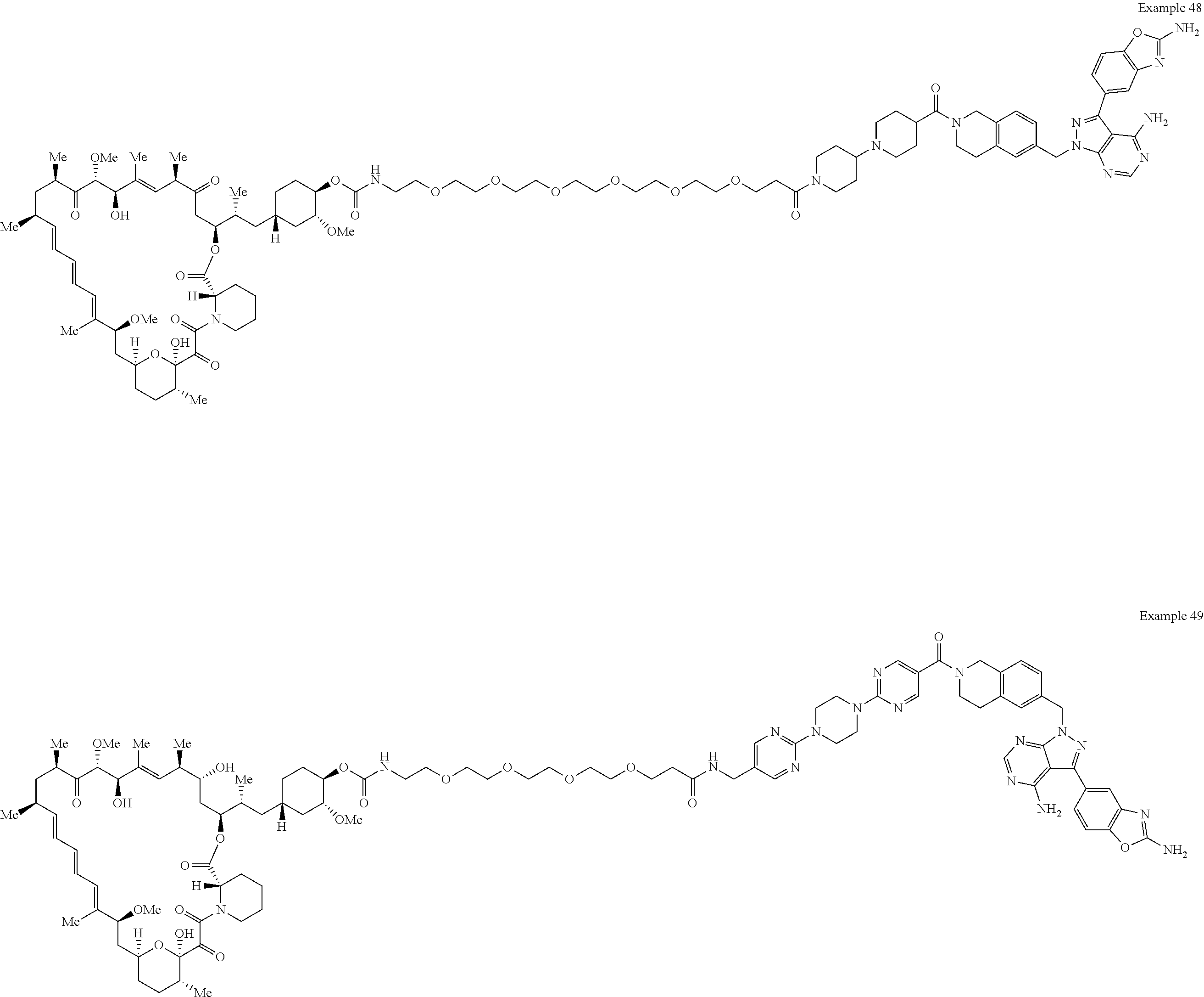

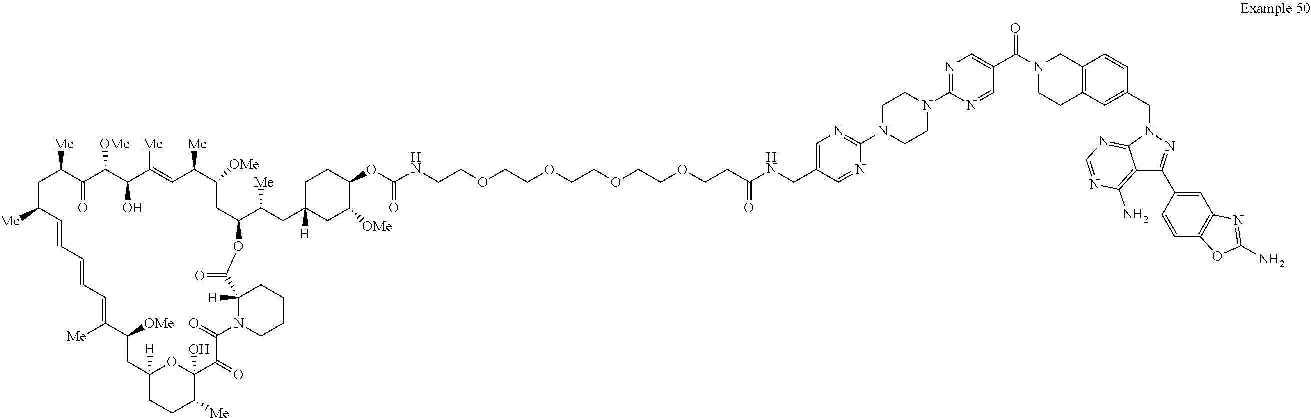 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

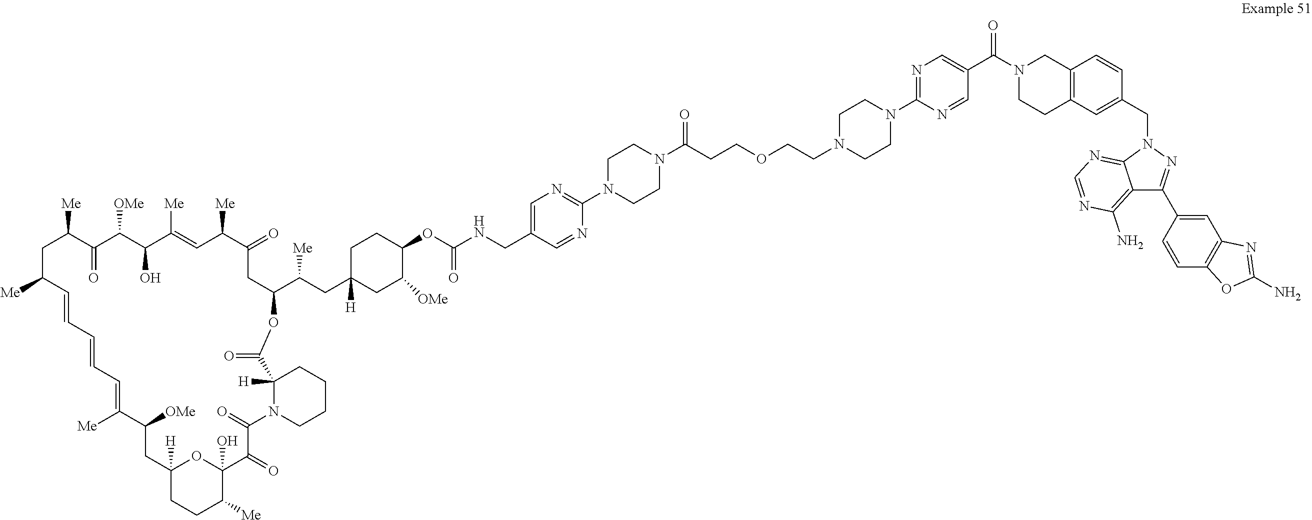 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

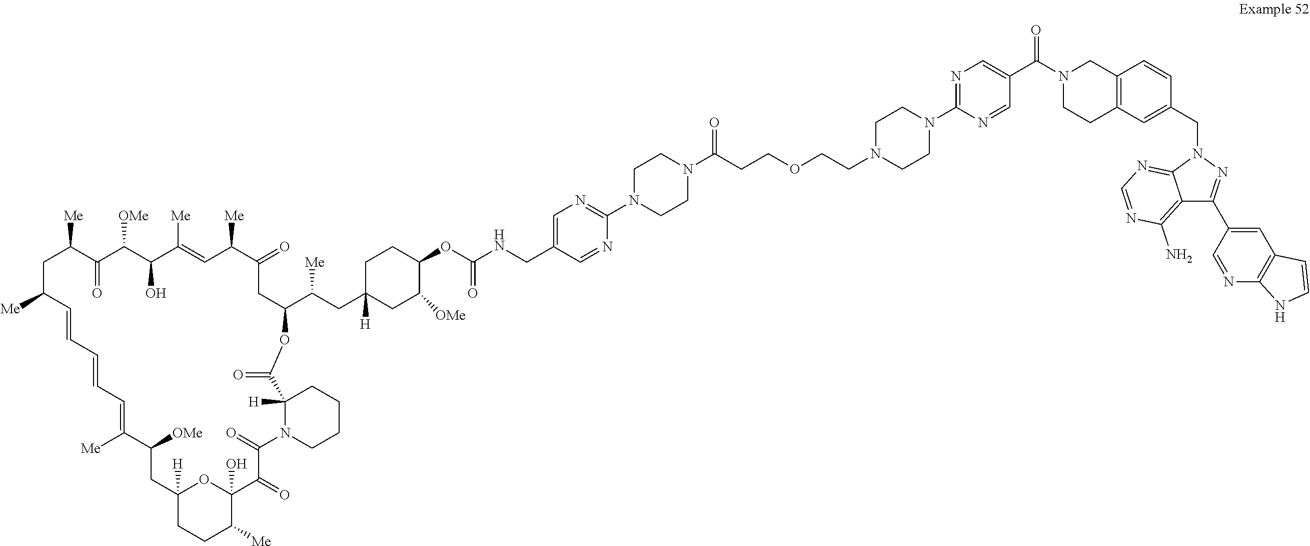 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

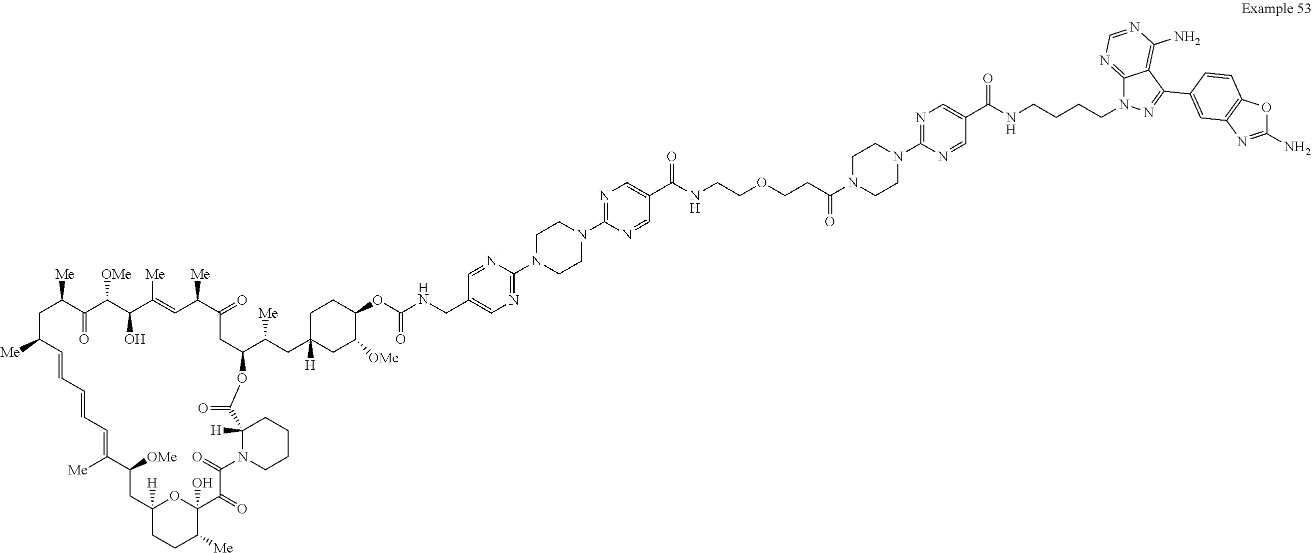 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCC(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

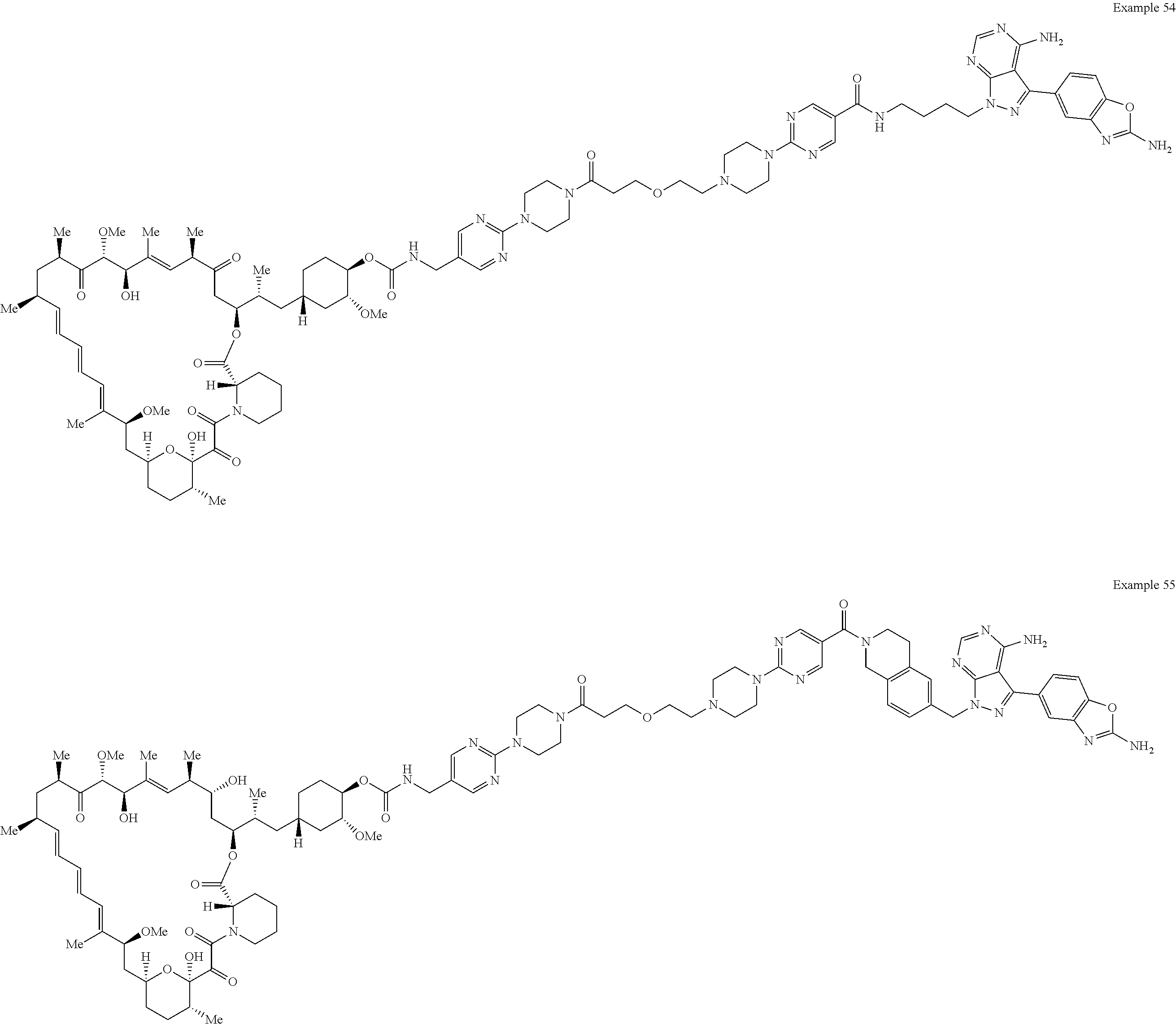

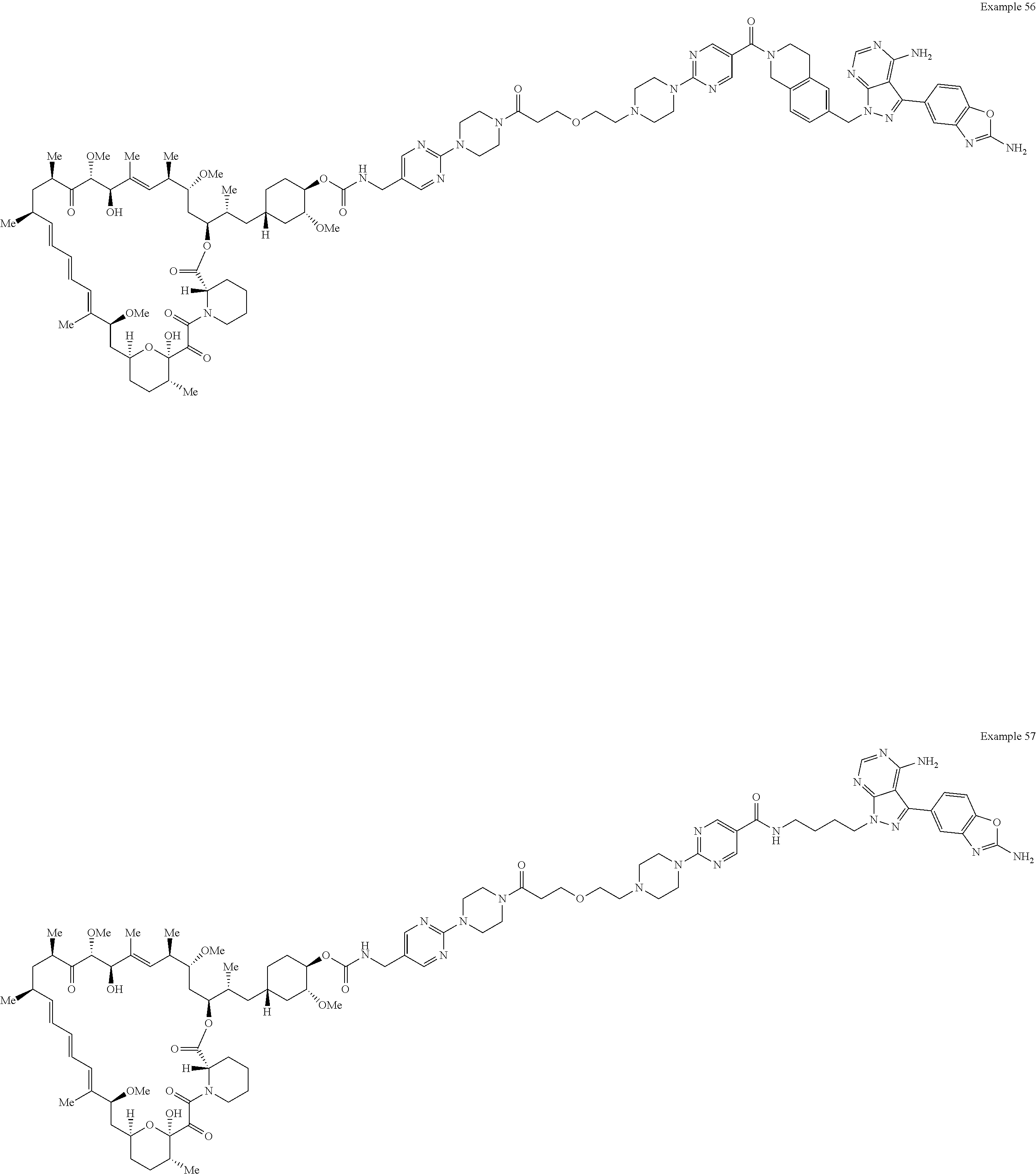

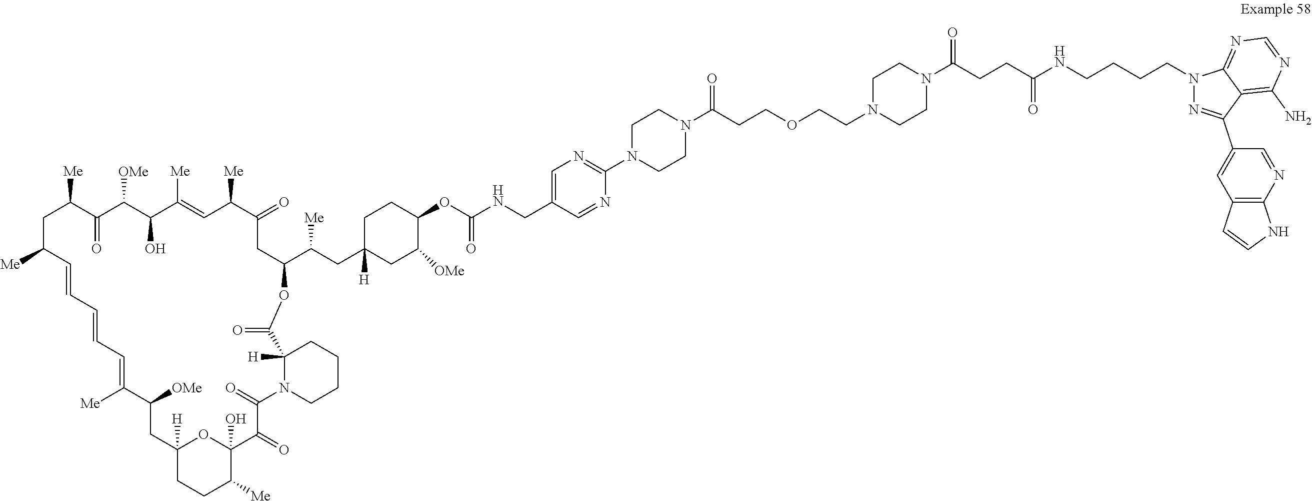 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)NCCCCn6nc(-c7cnc8[nH]ccc8c7)c7c(N)ncnc76)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

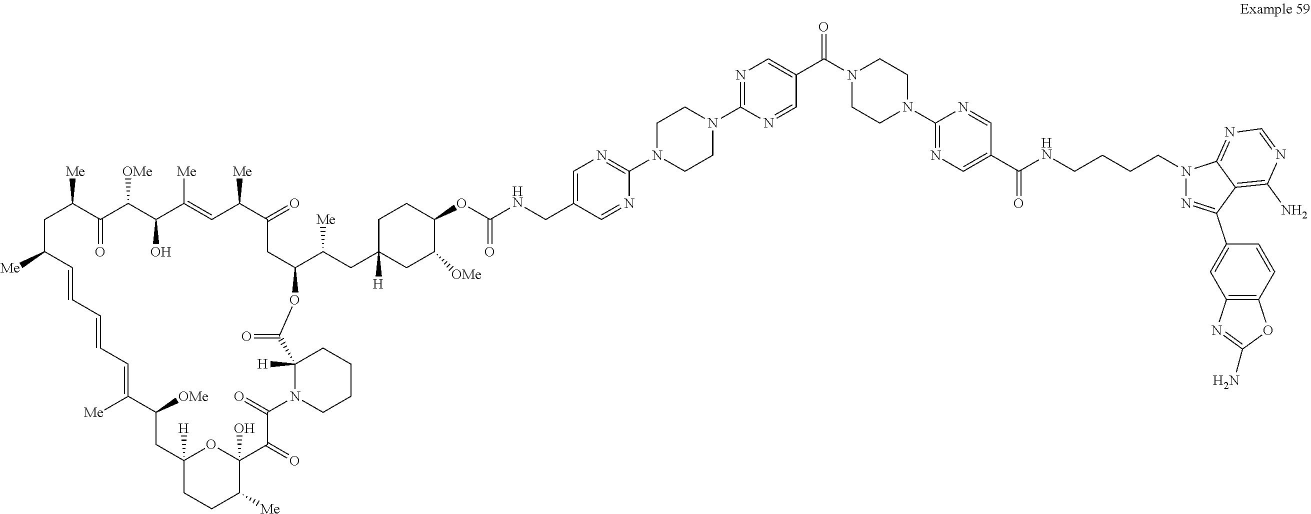 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

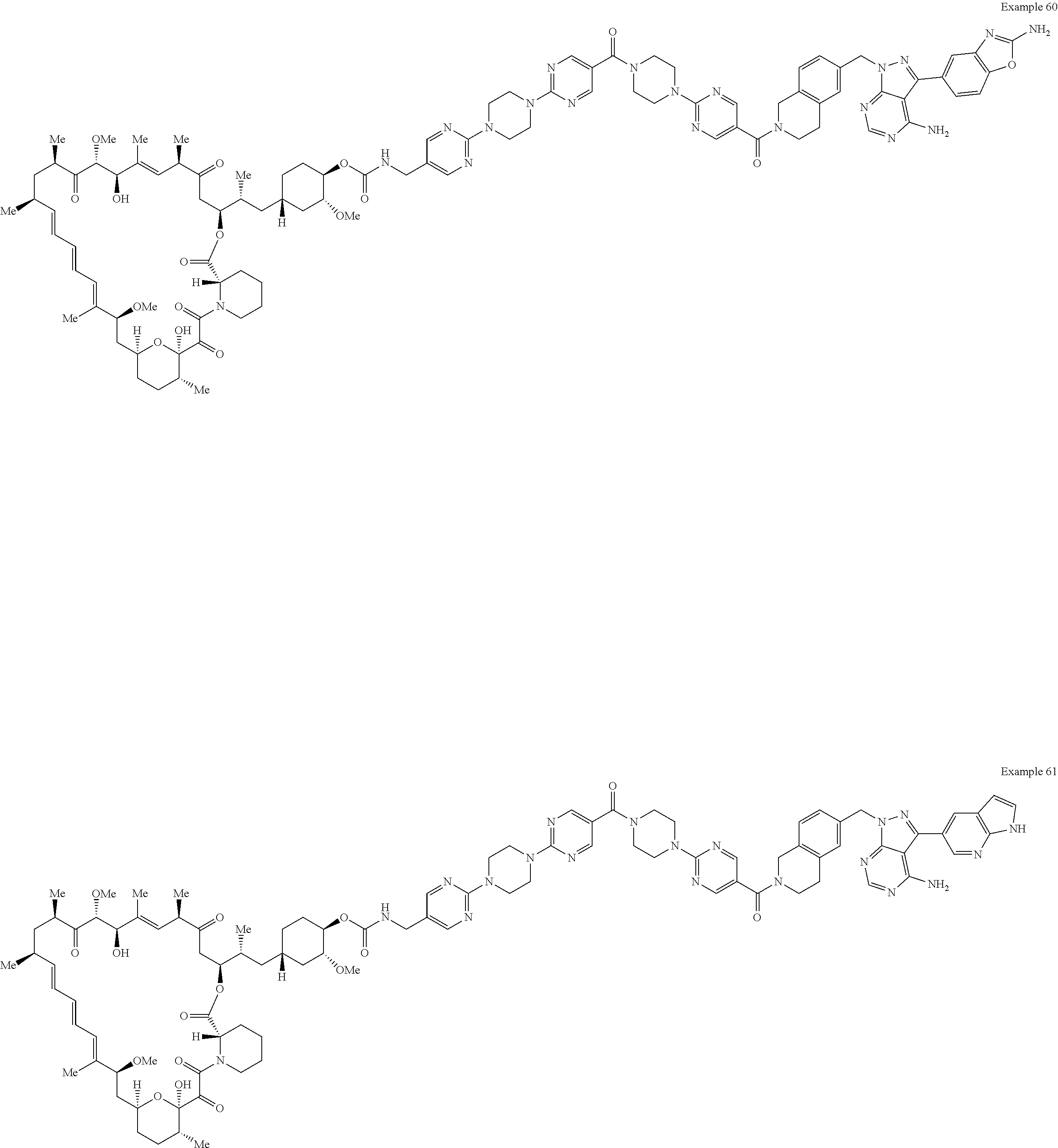

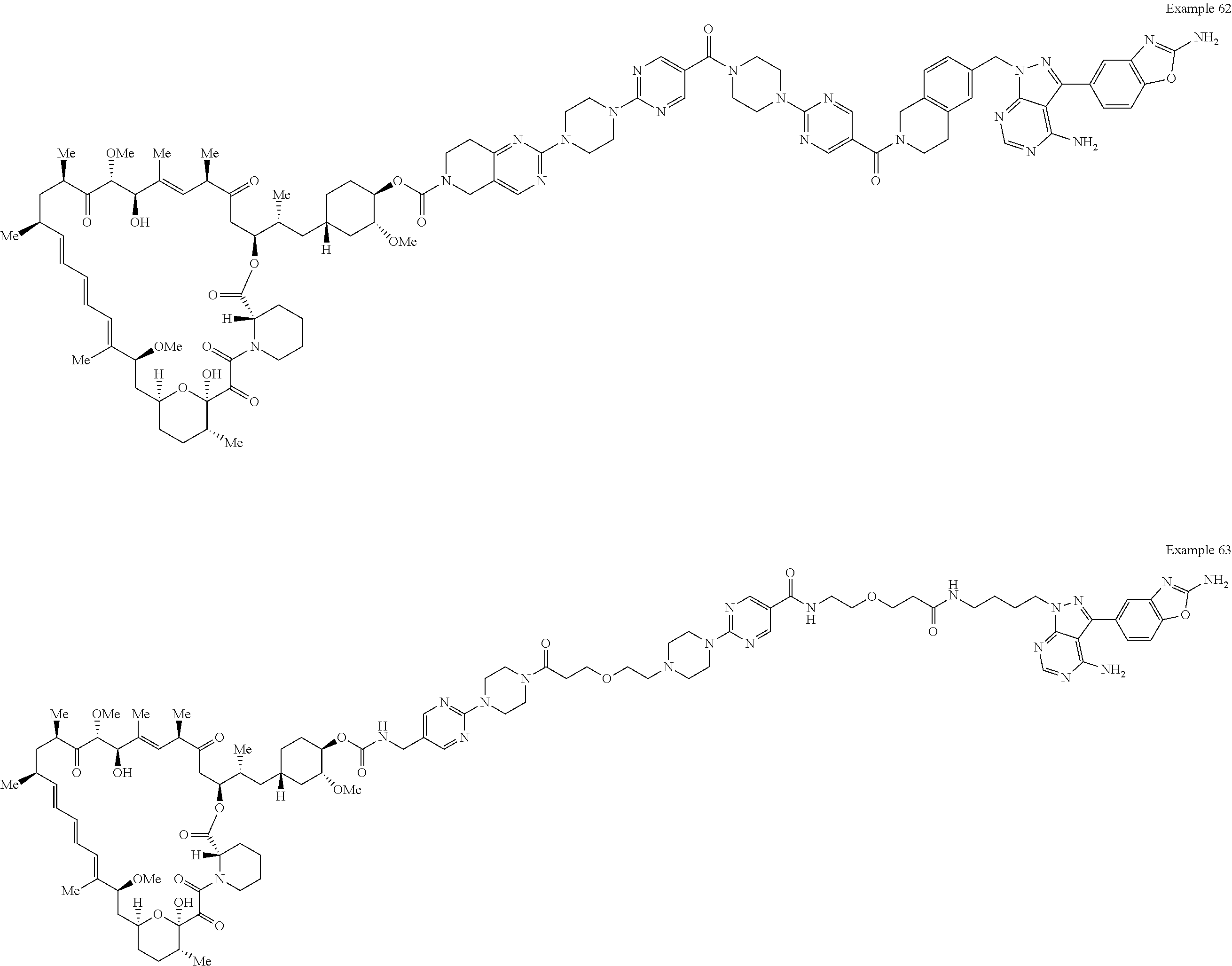

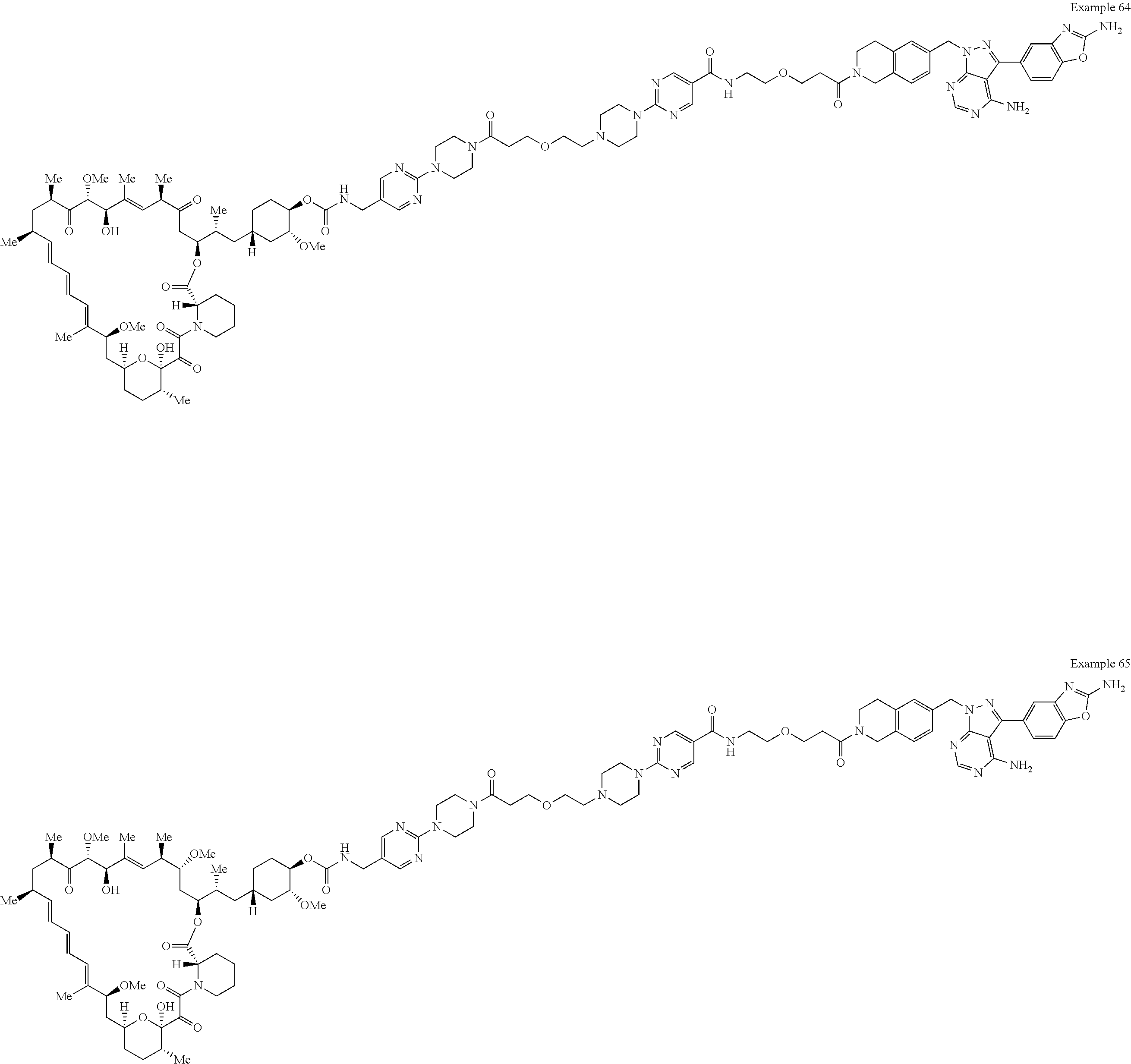

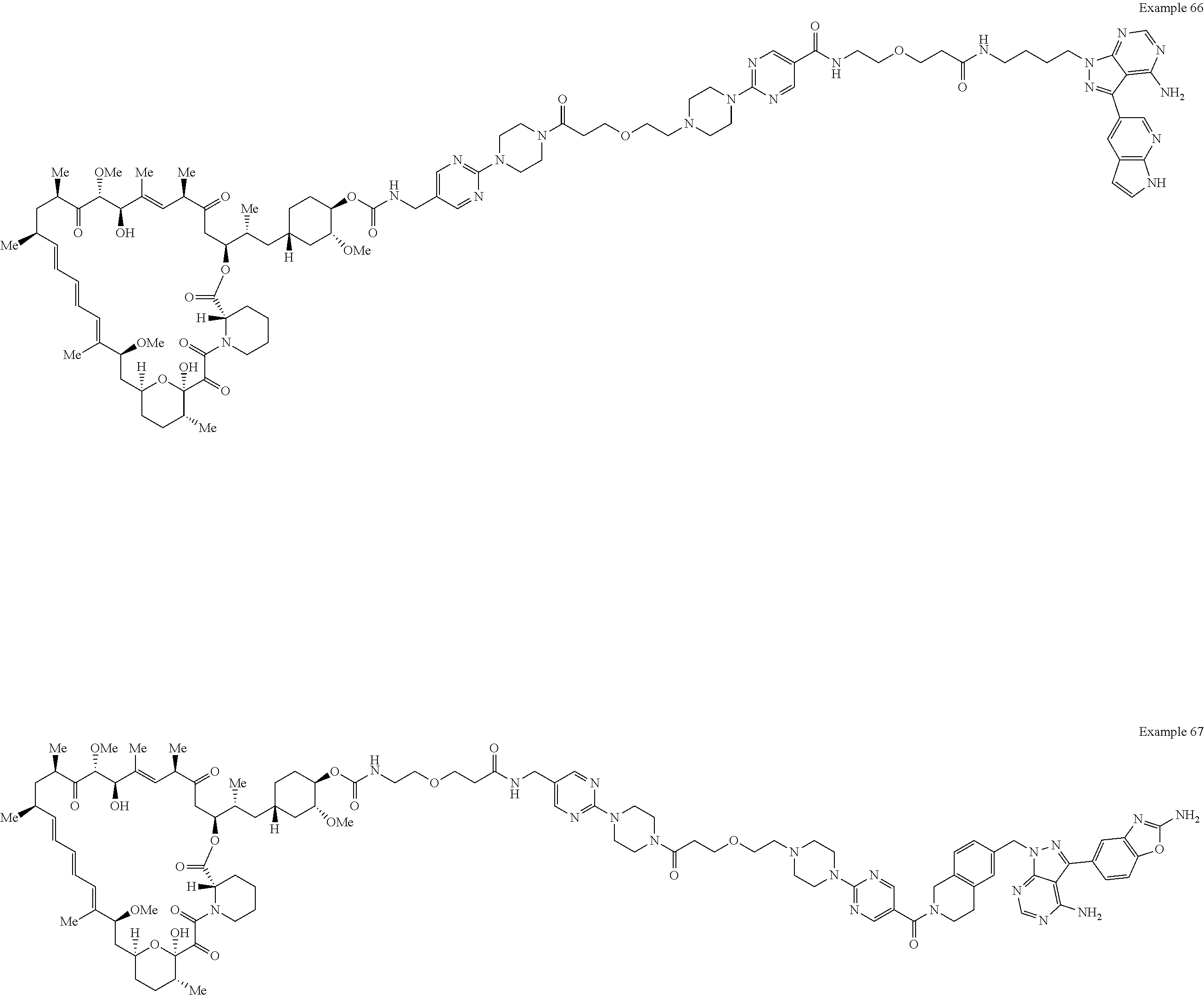

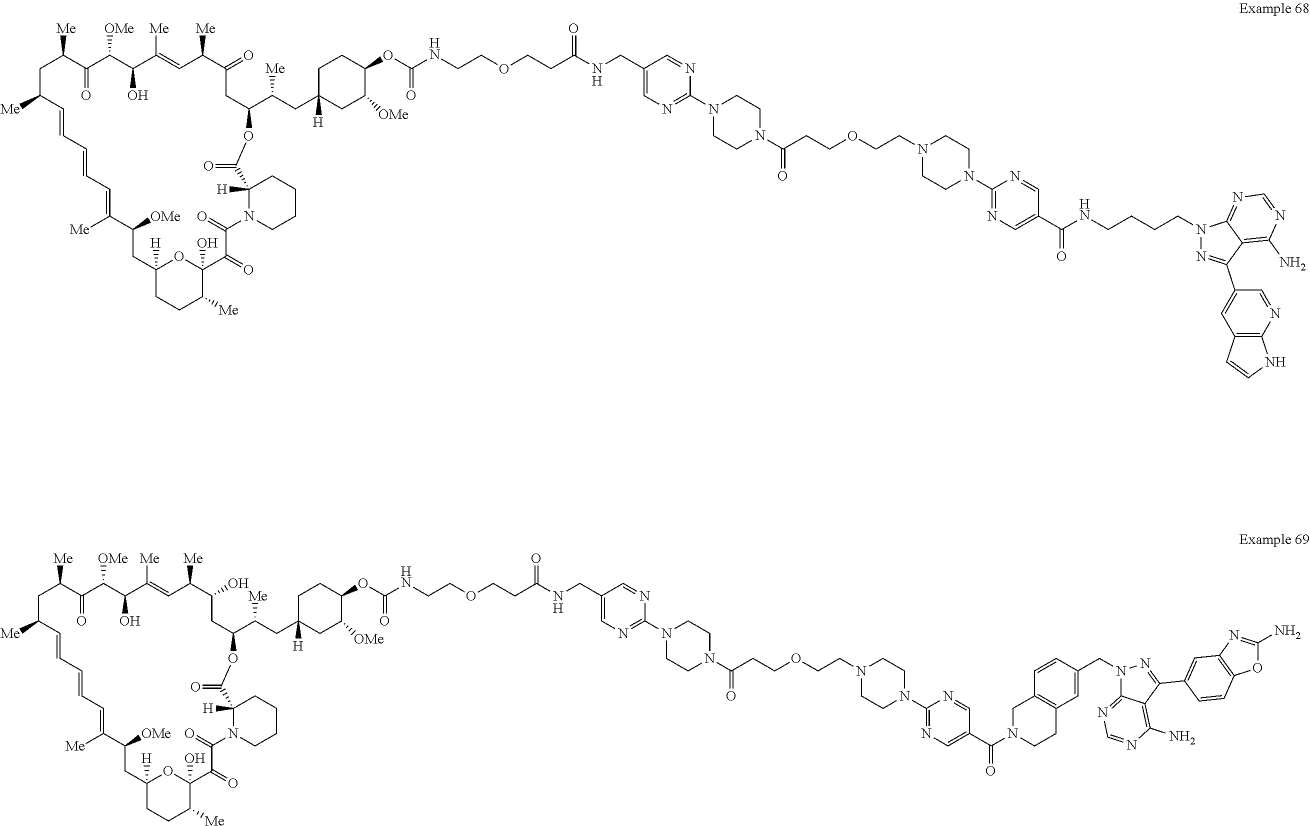

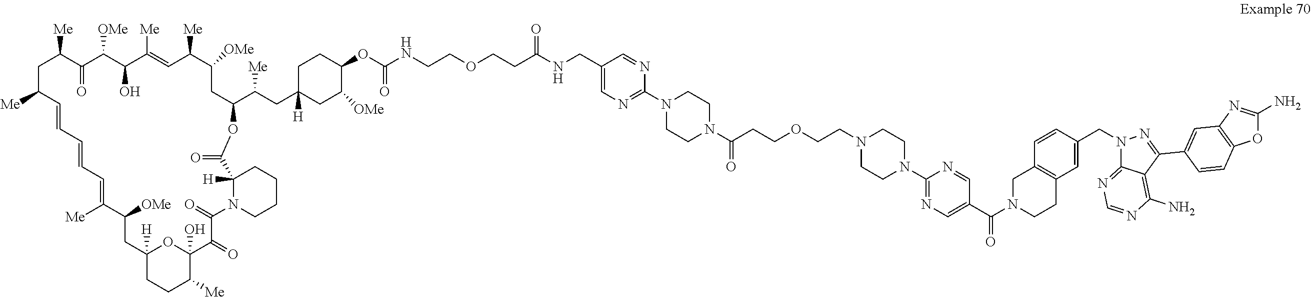 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

or a pharmaceutically acceptable salt or tautomer thereof.

Embodiment II-73. A compound selected from the group consisting of:

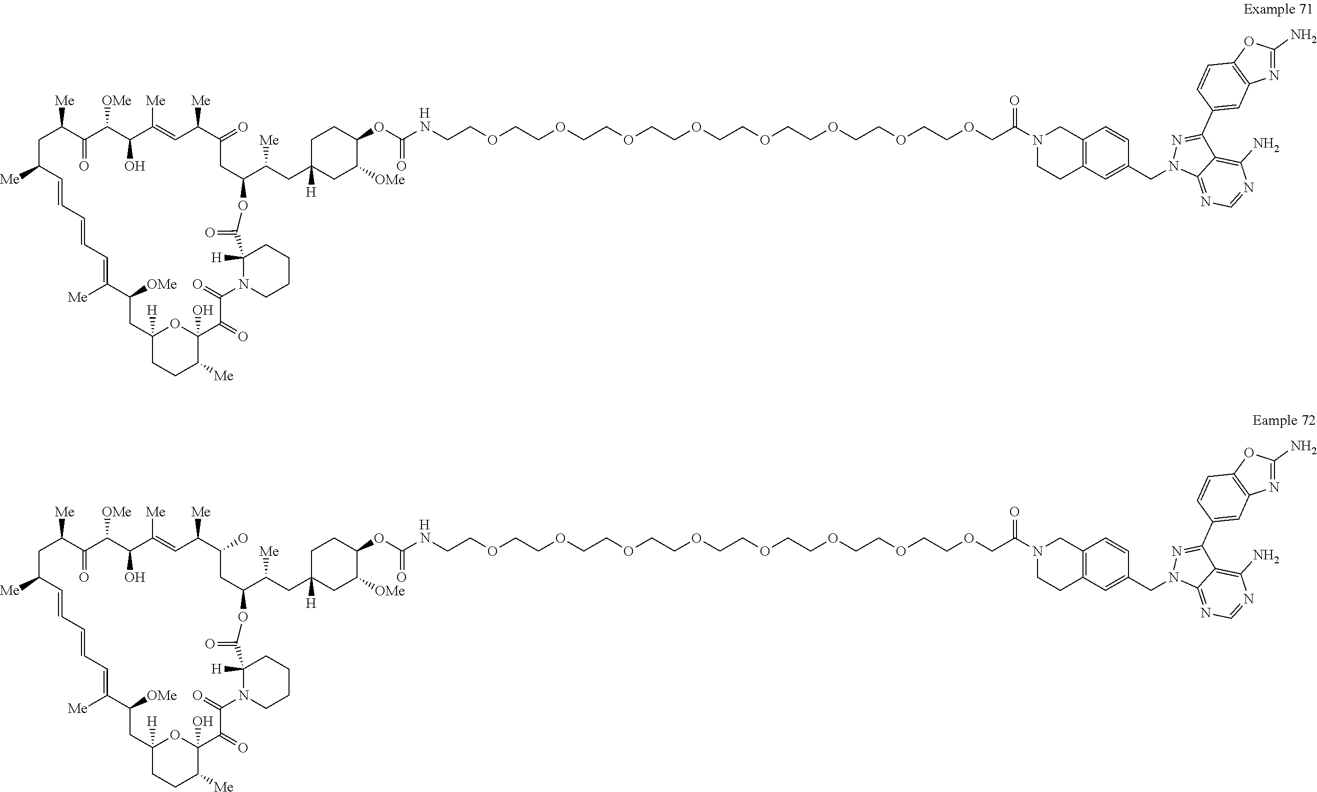

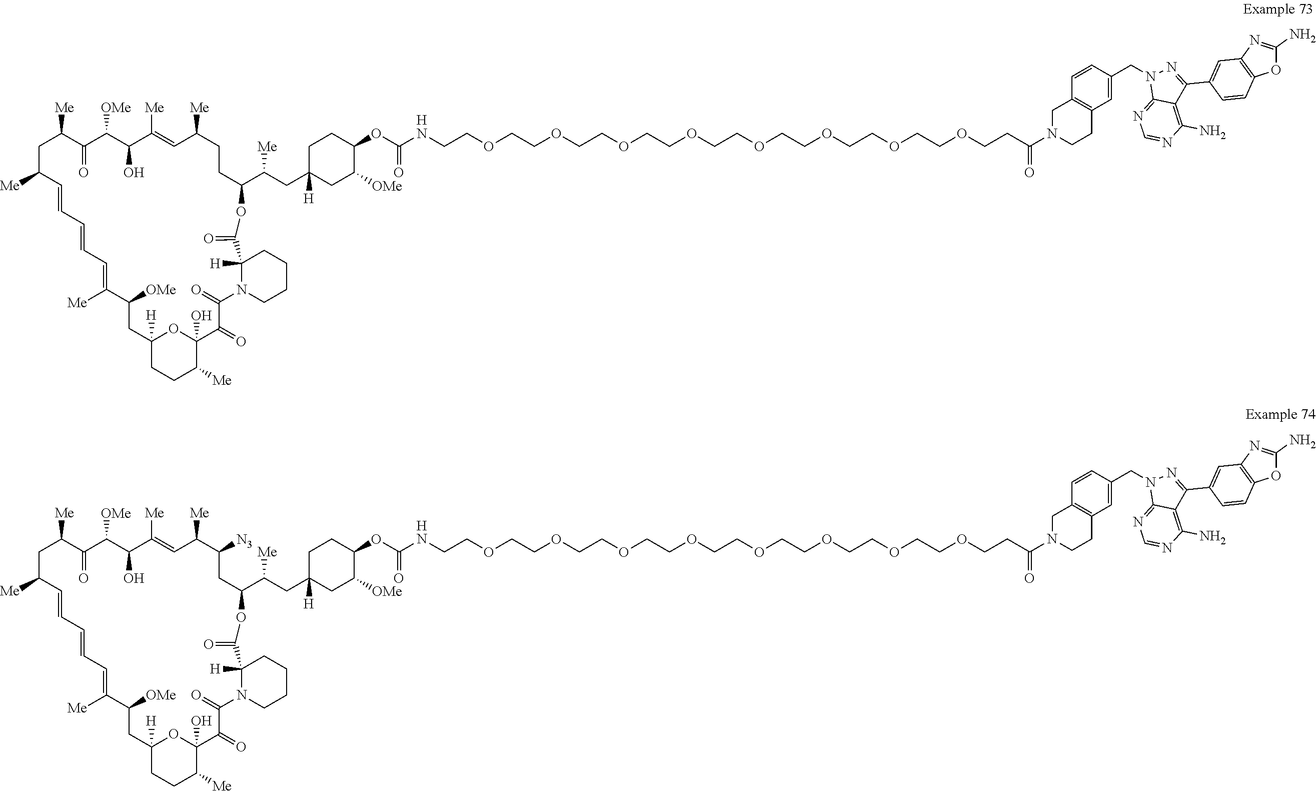

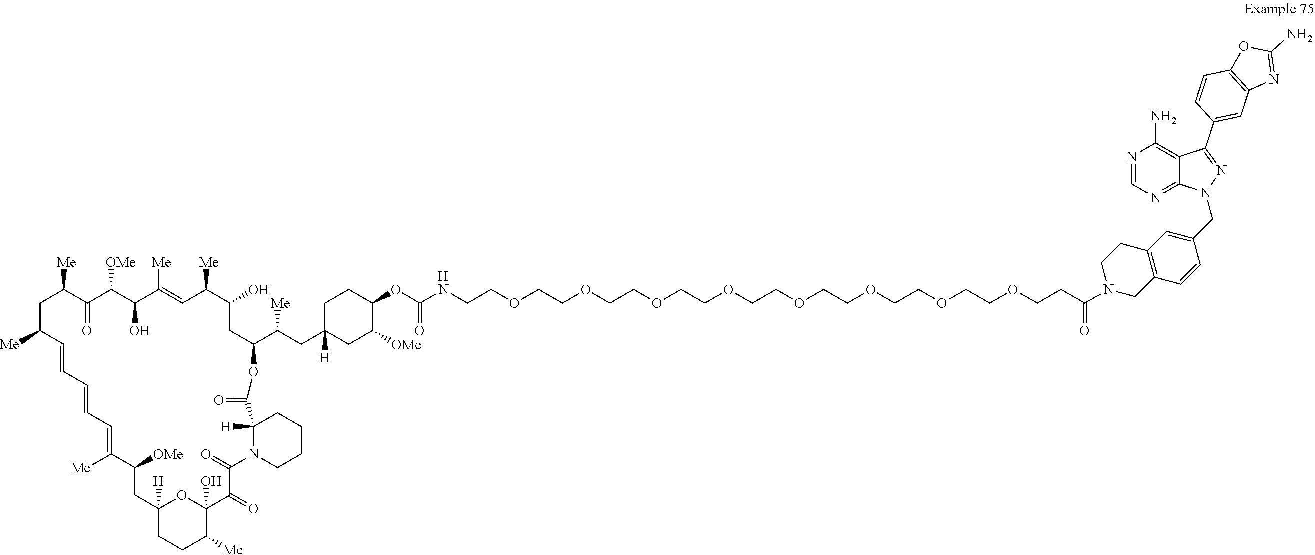

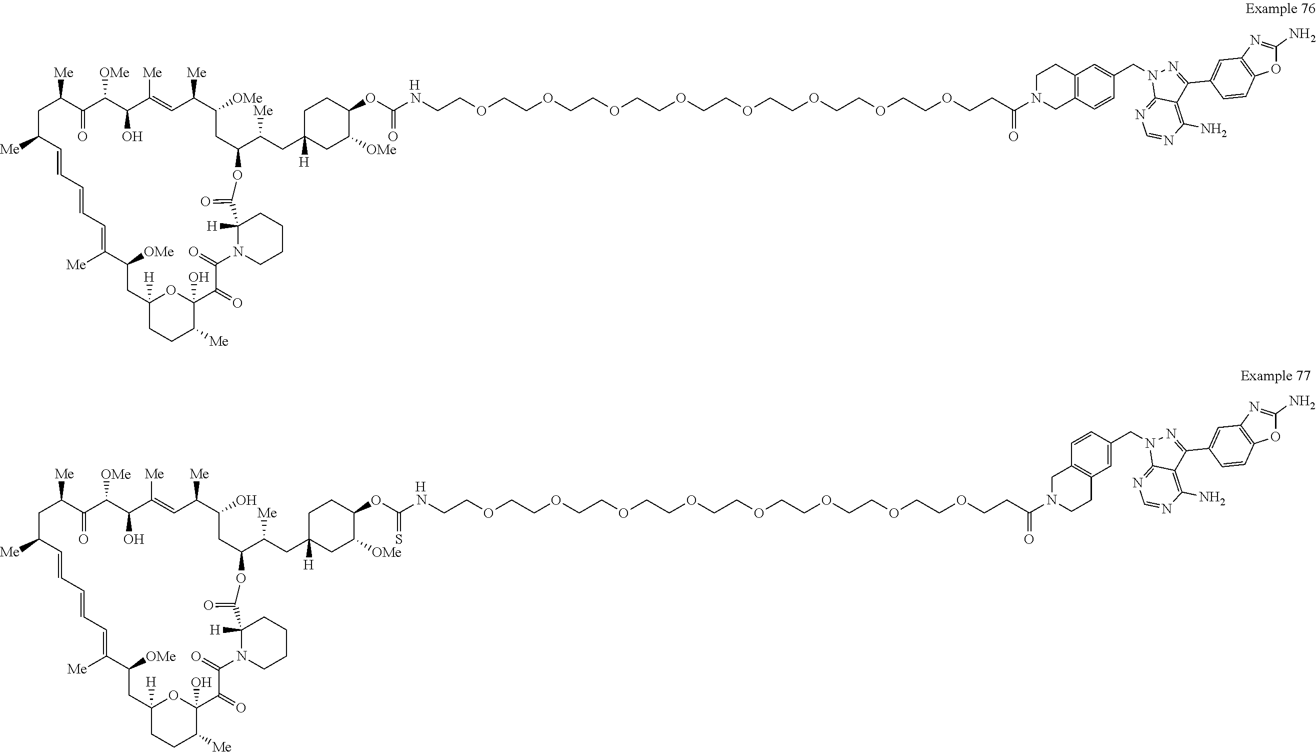

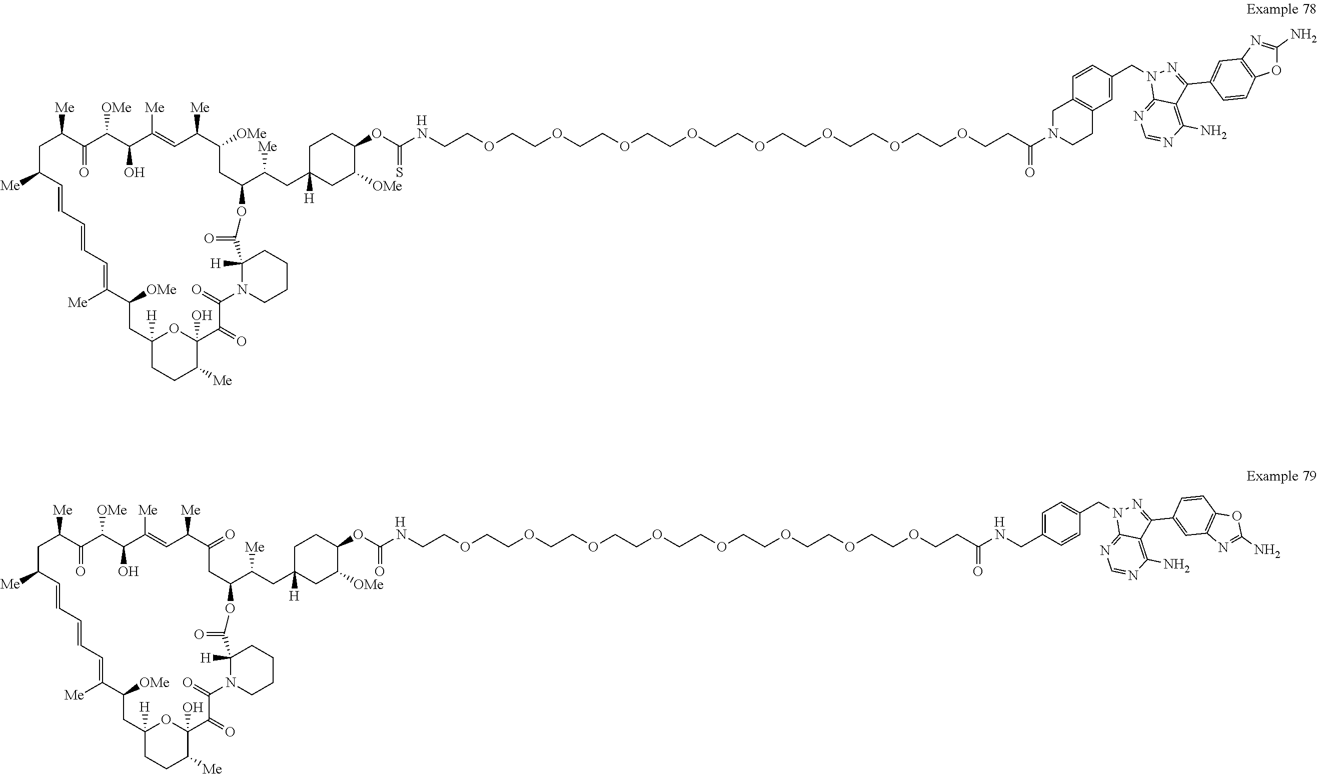

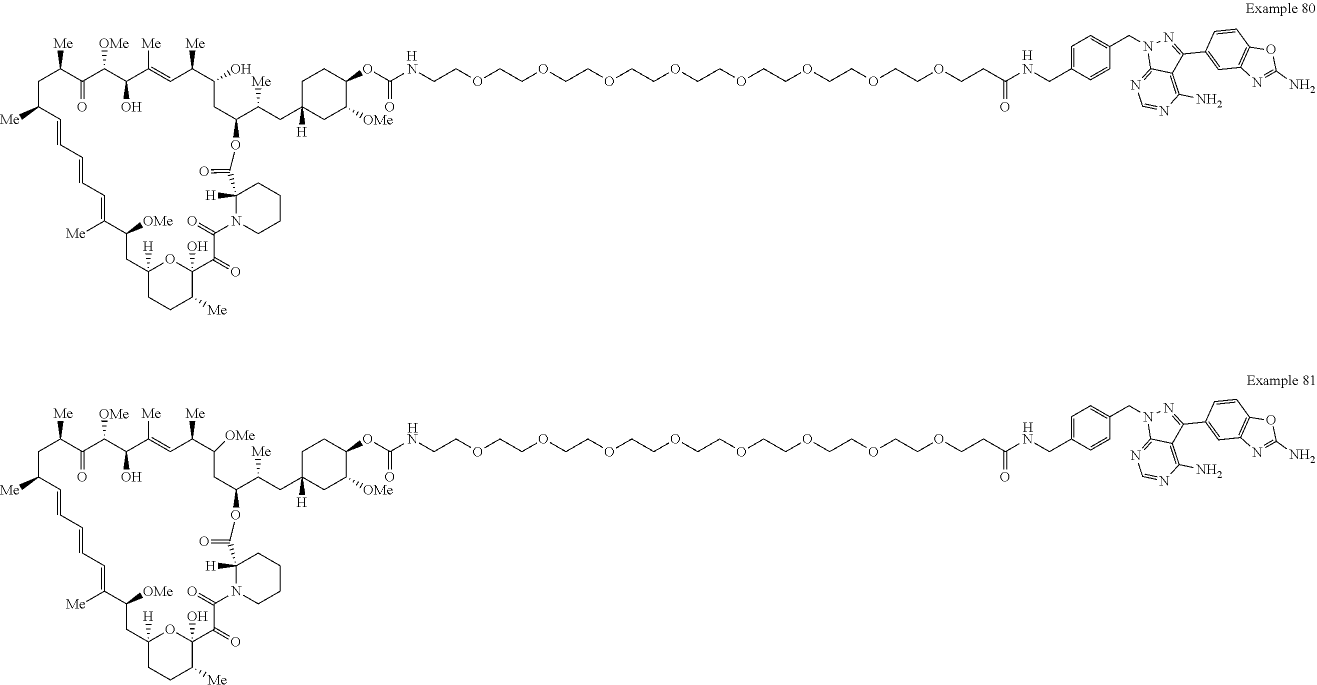

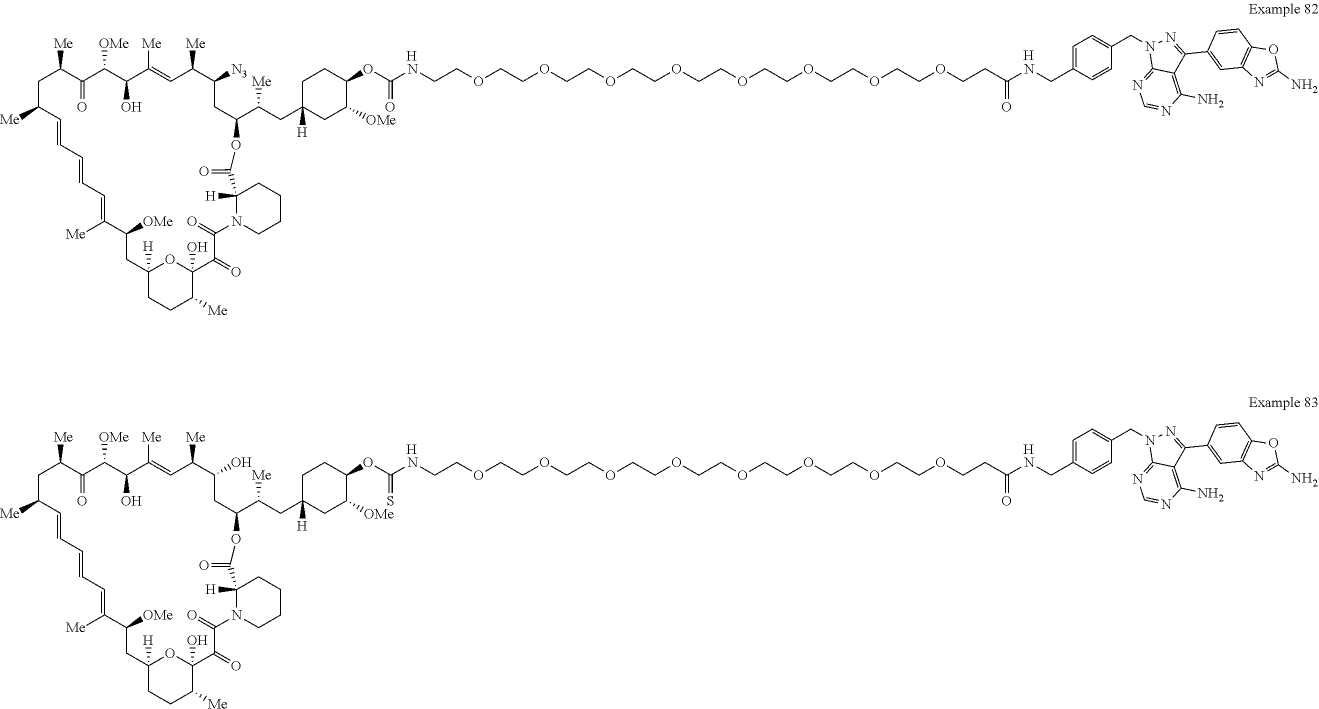

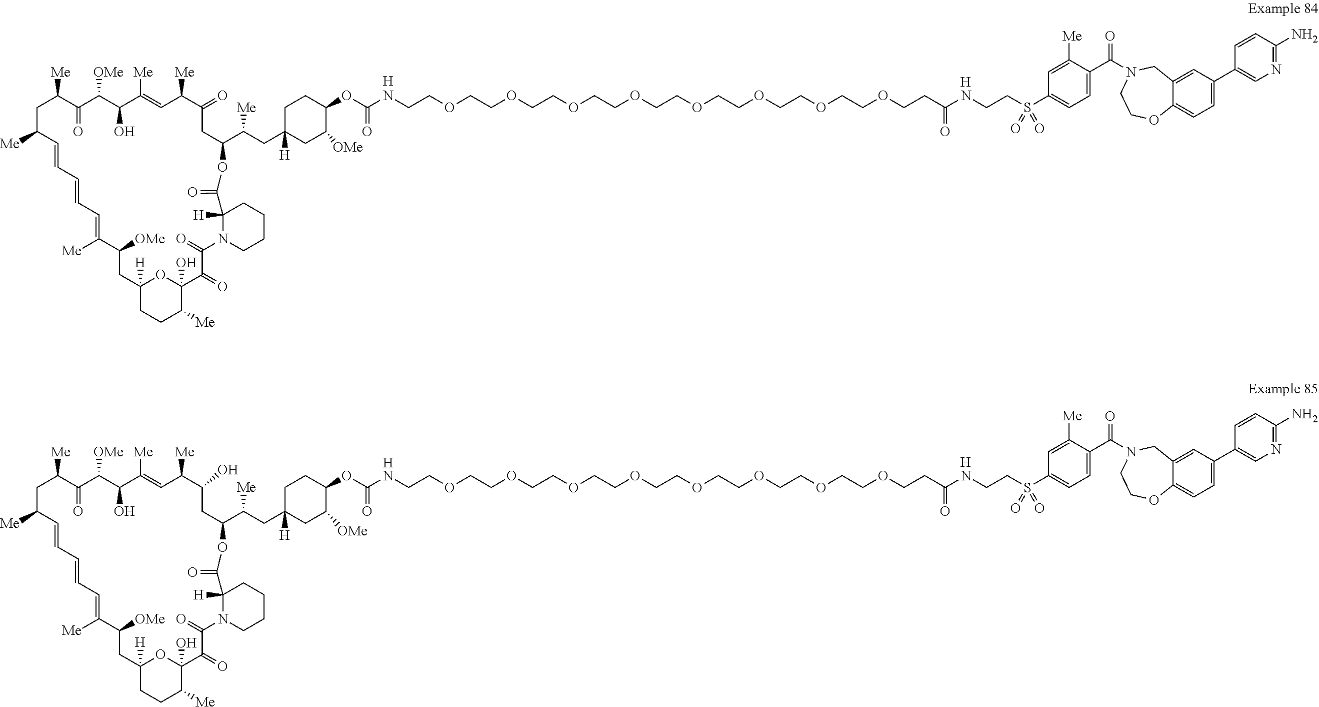

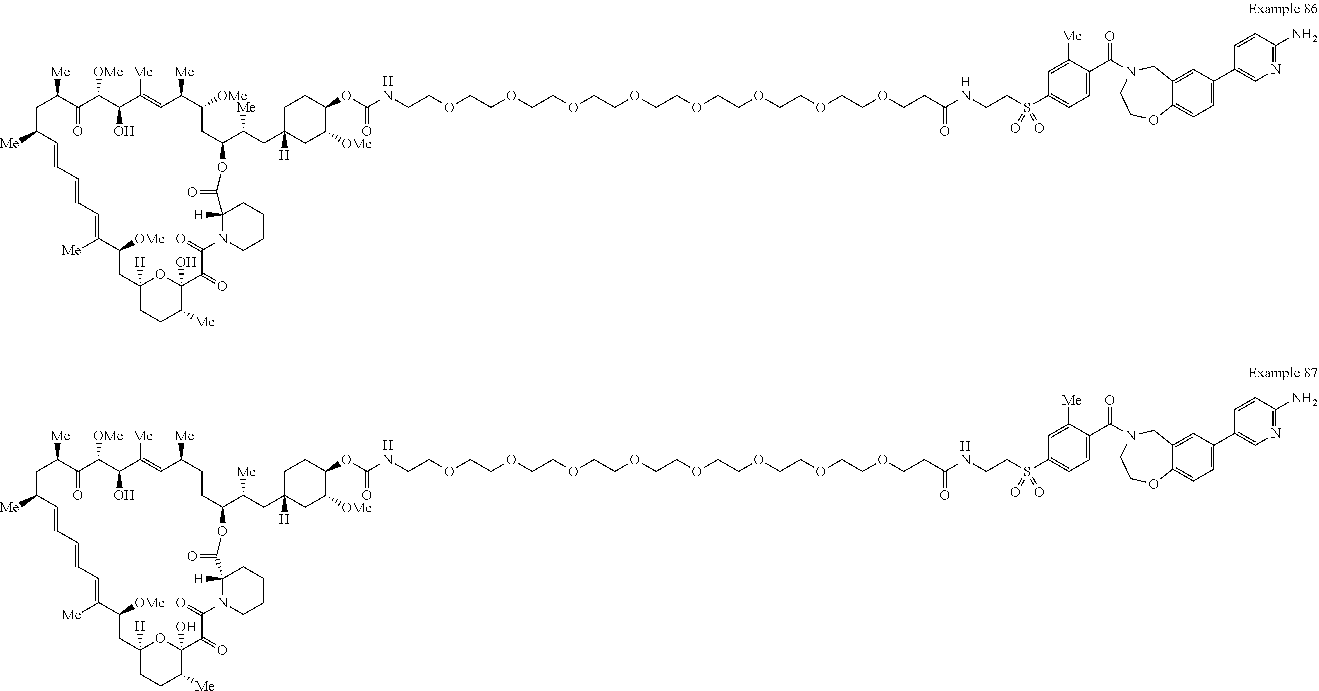

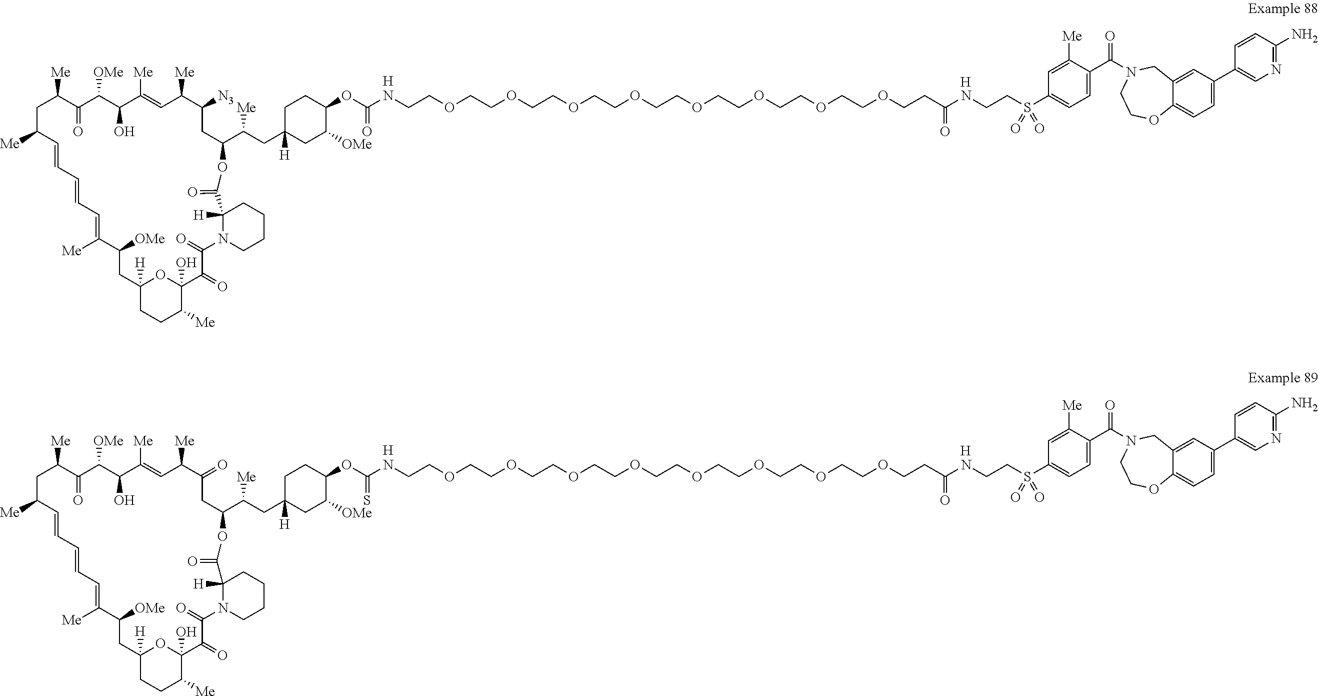

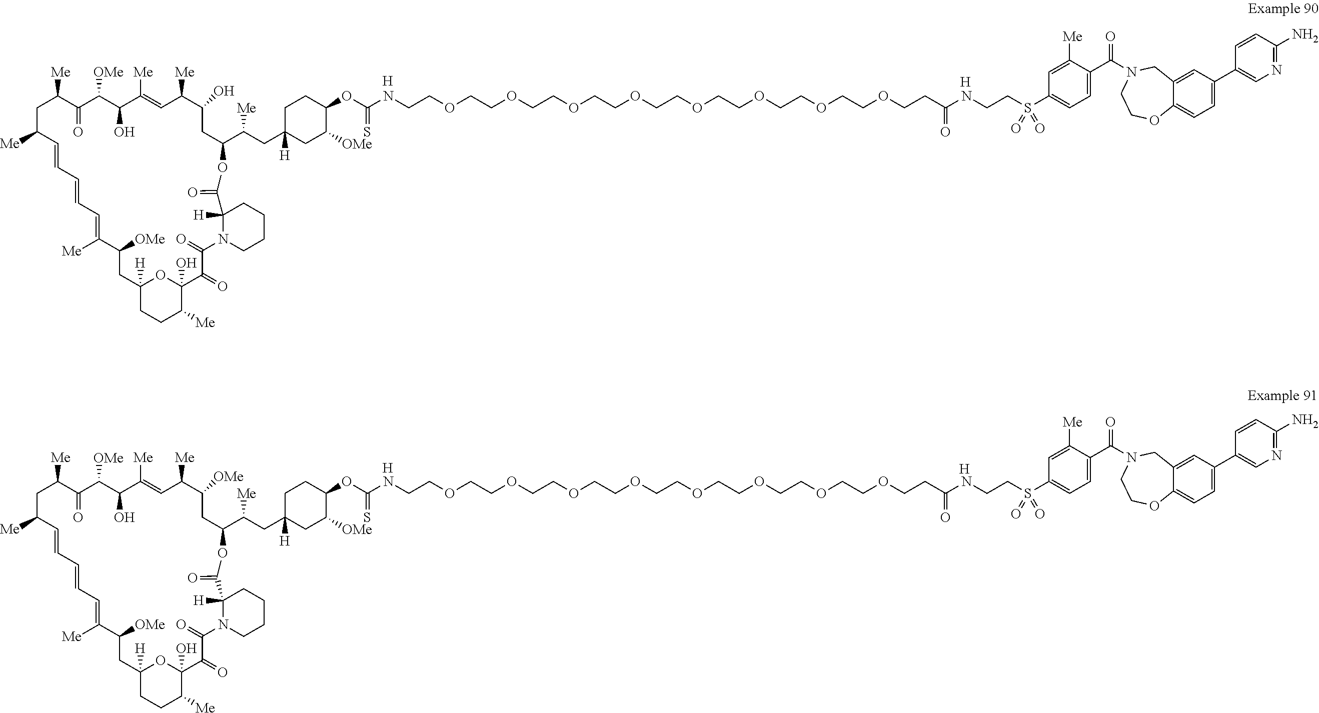

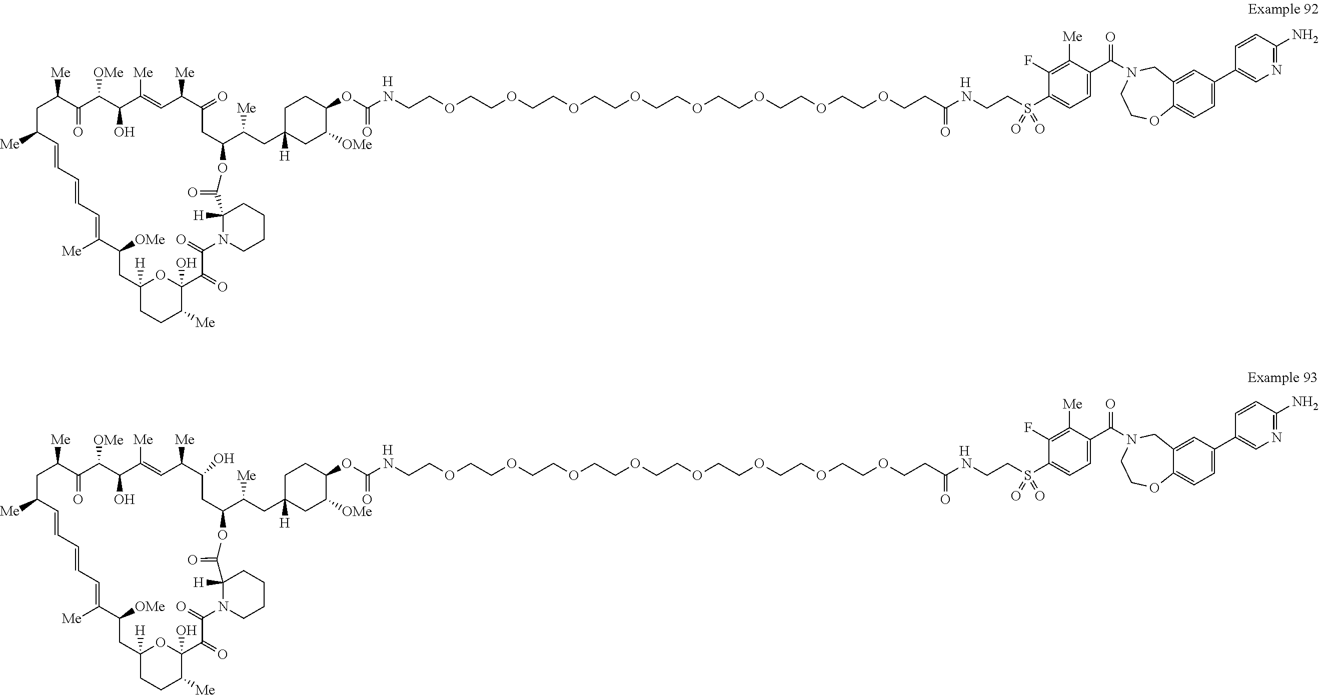

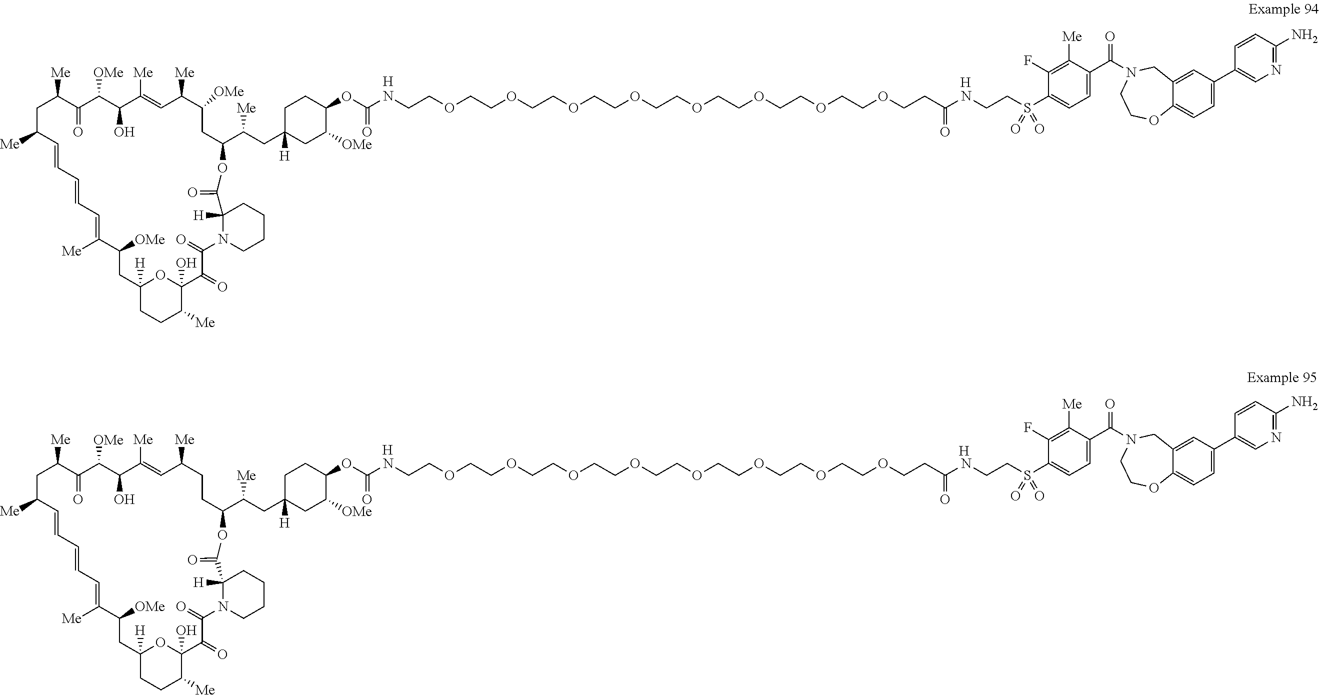

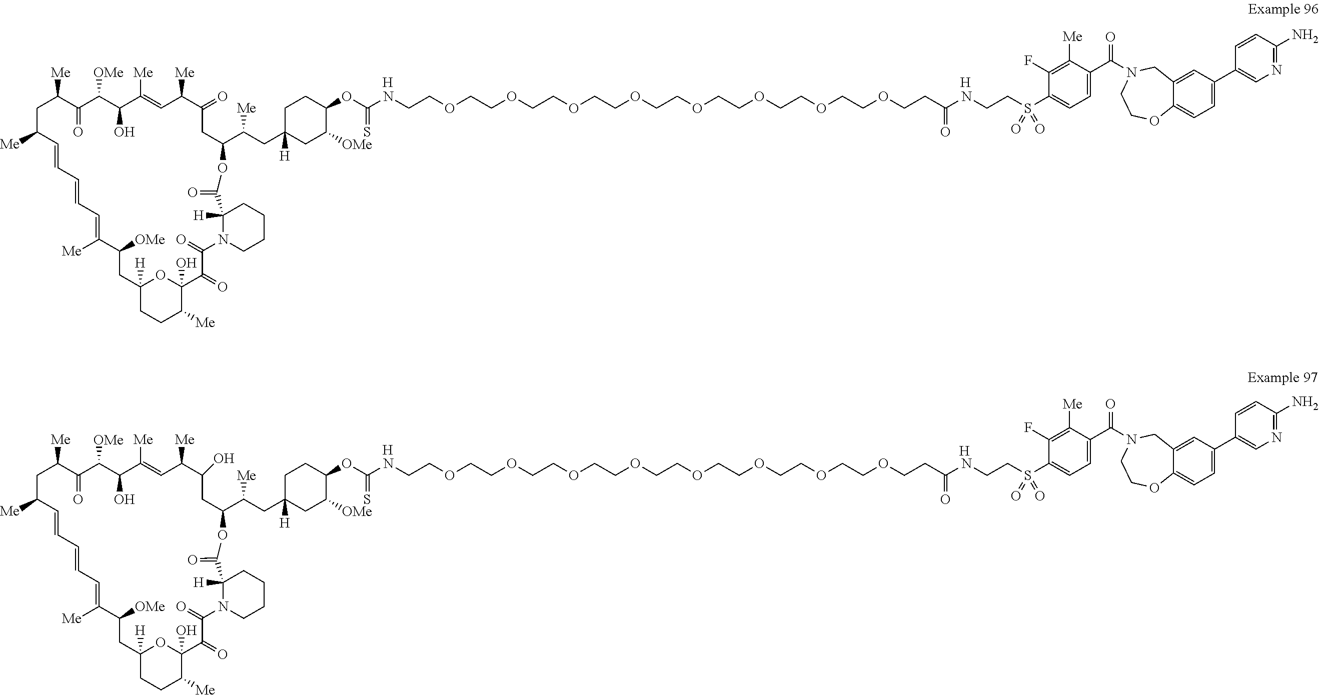

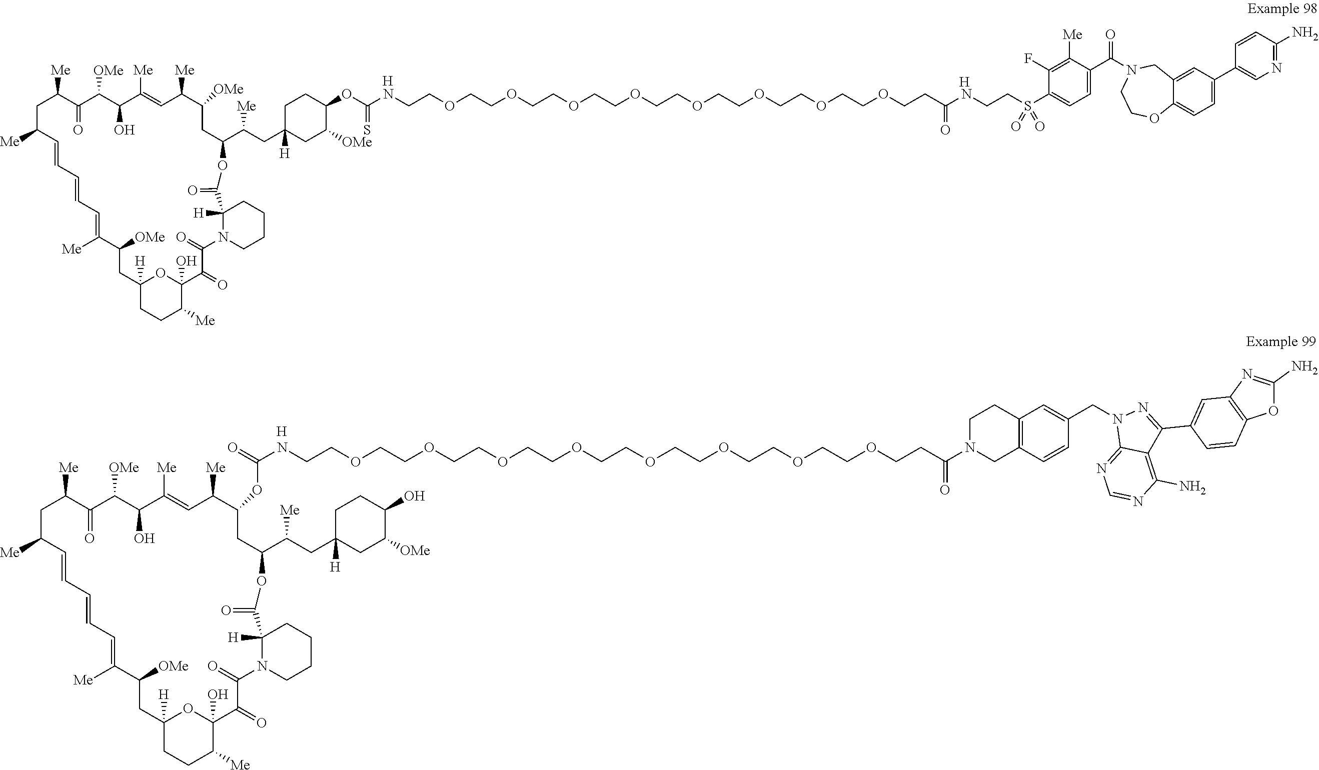

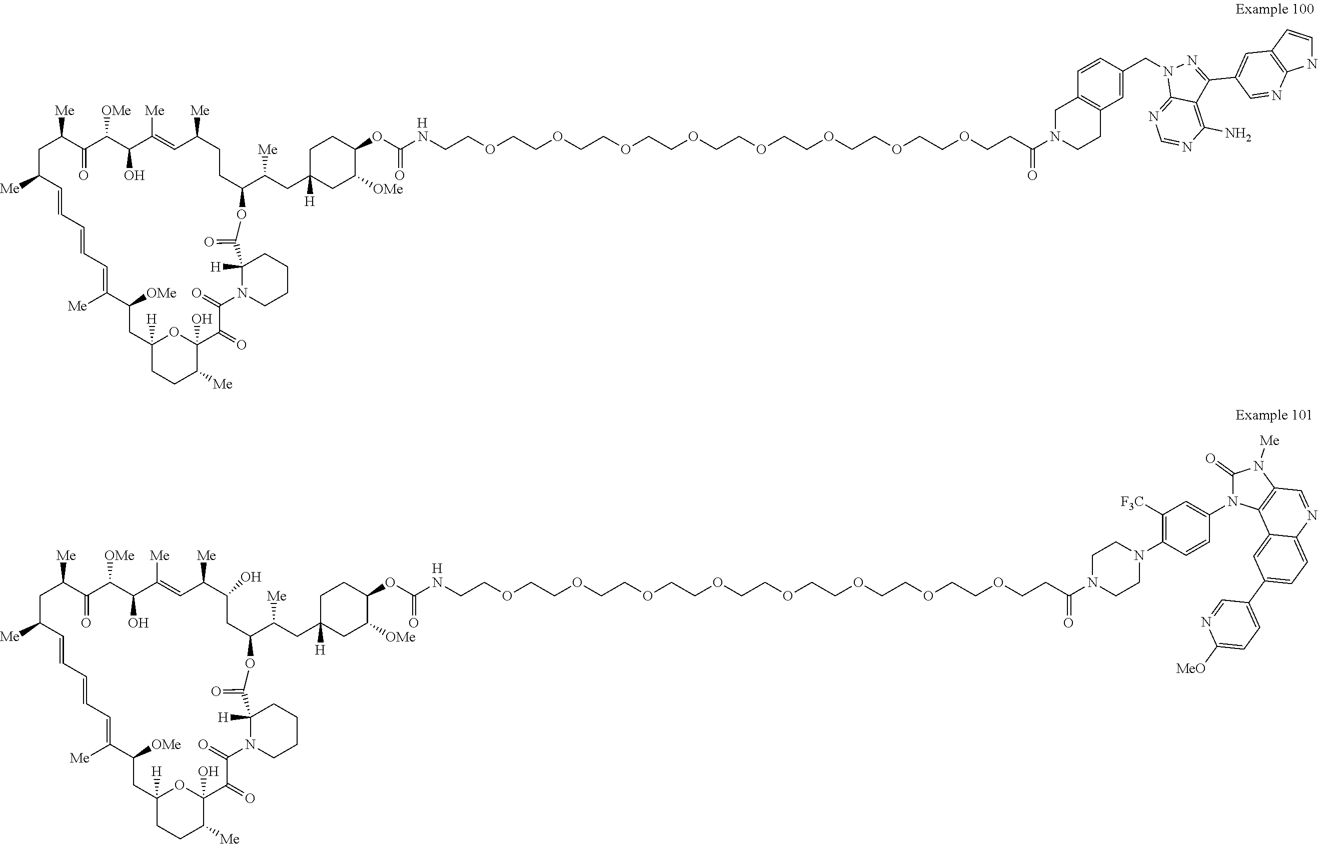

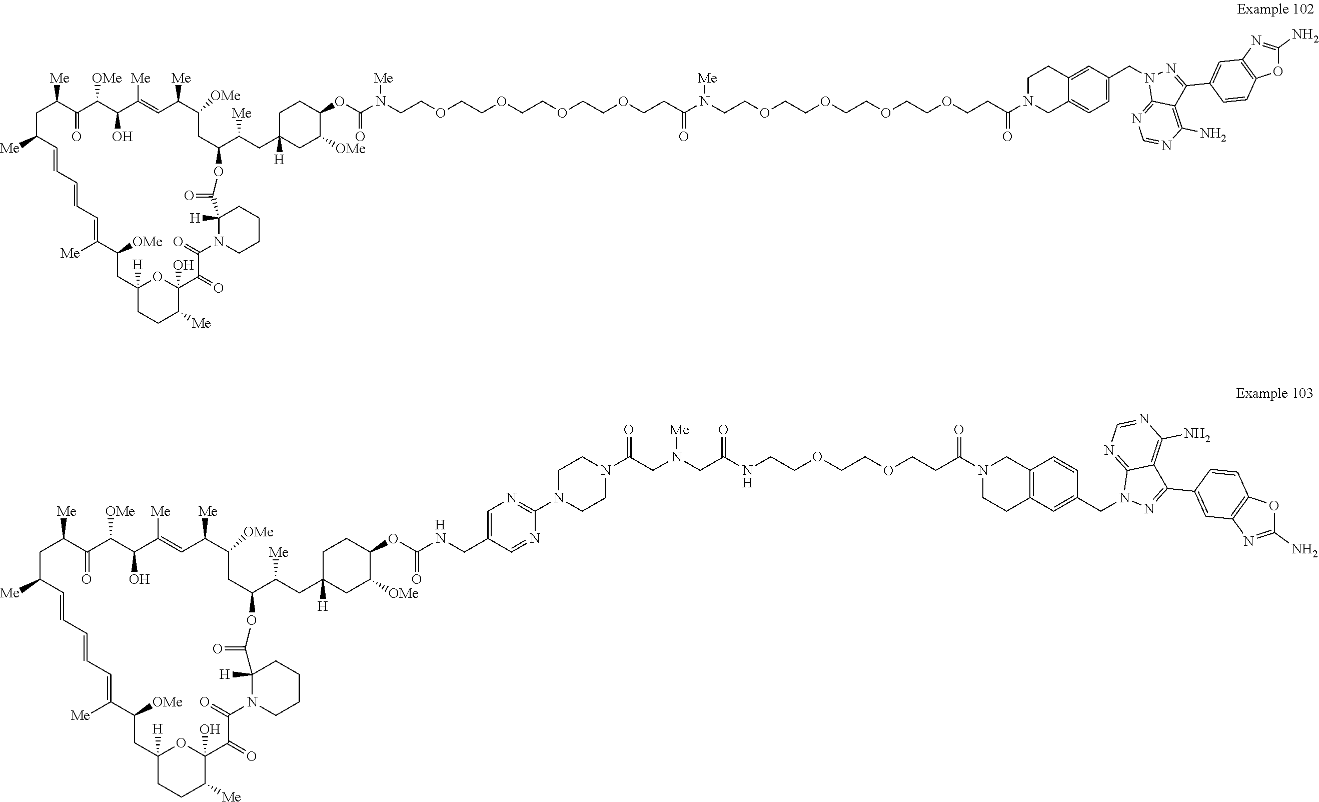

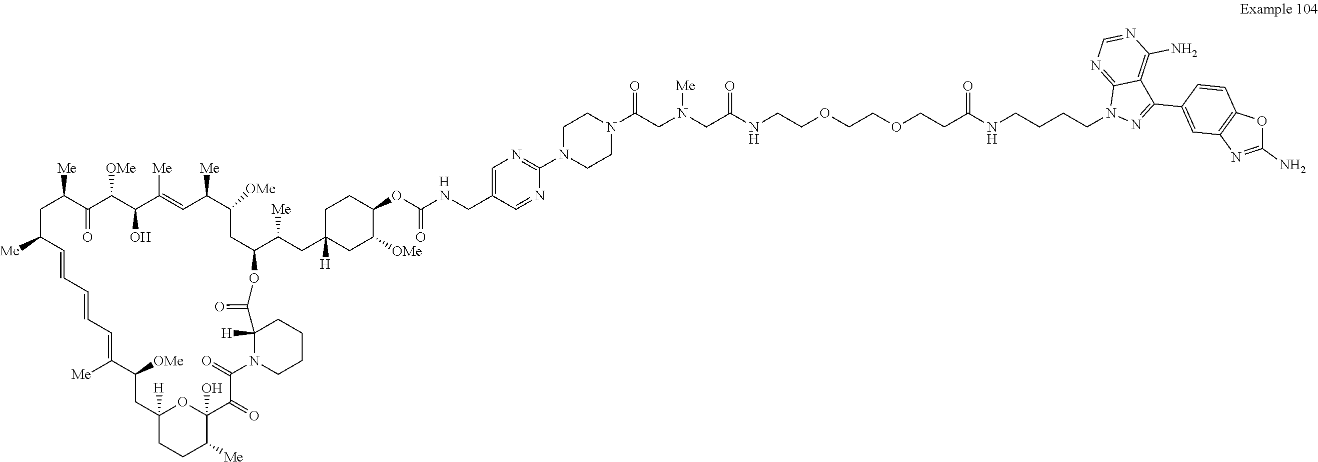

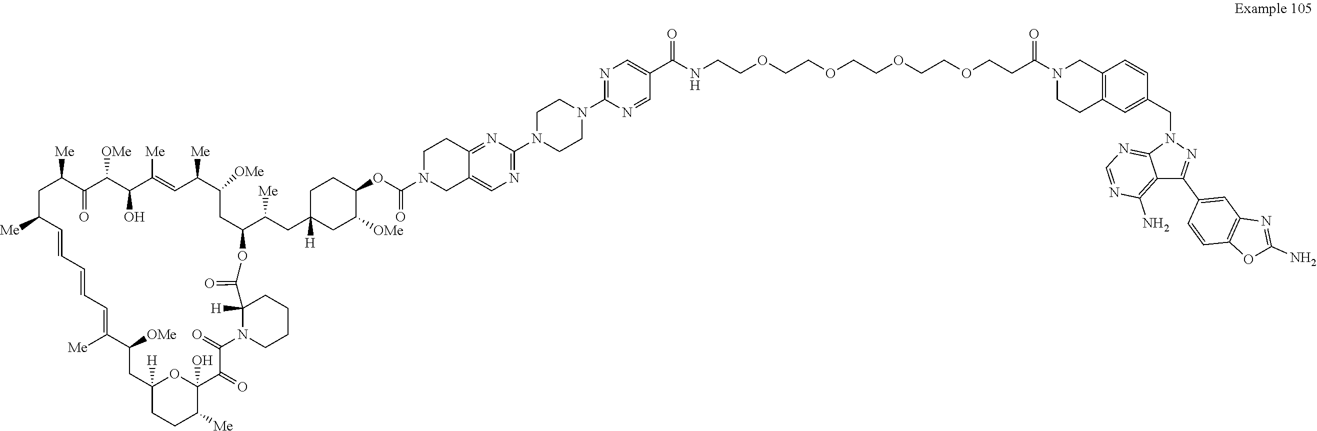 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

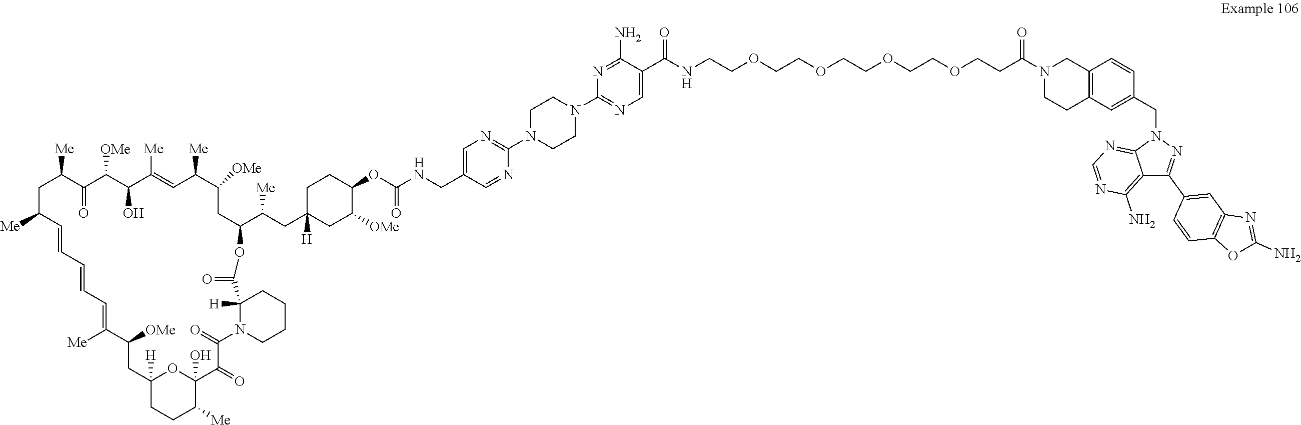

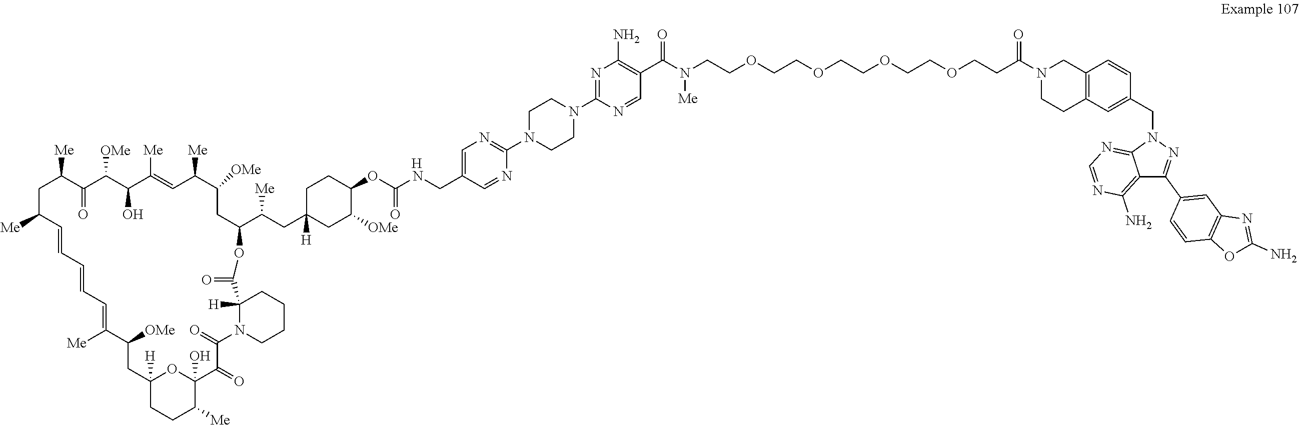

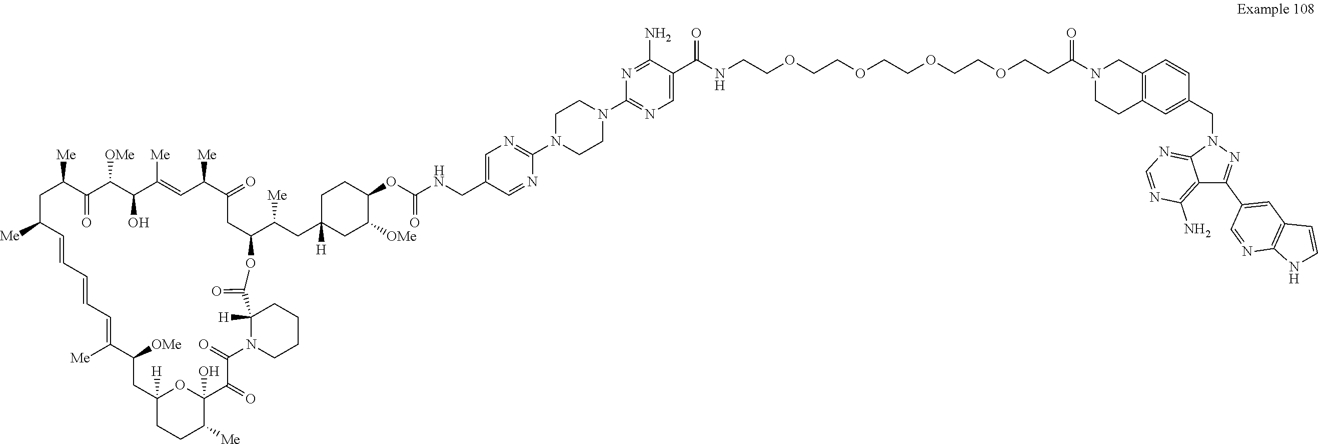

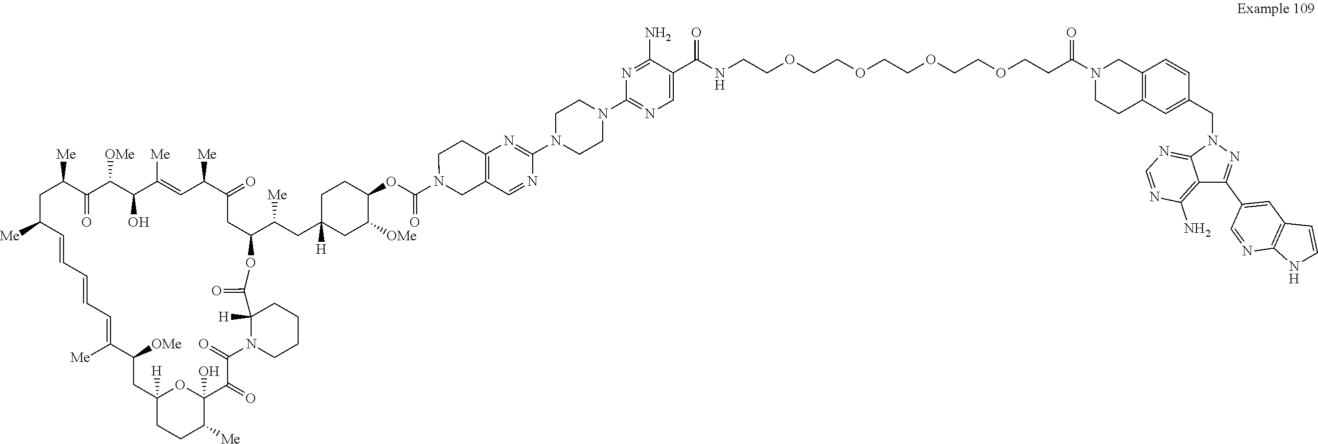 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)c(N)n6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

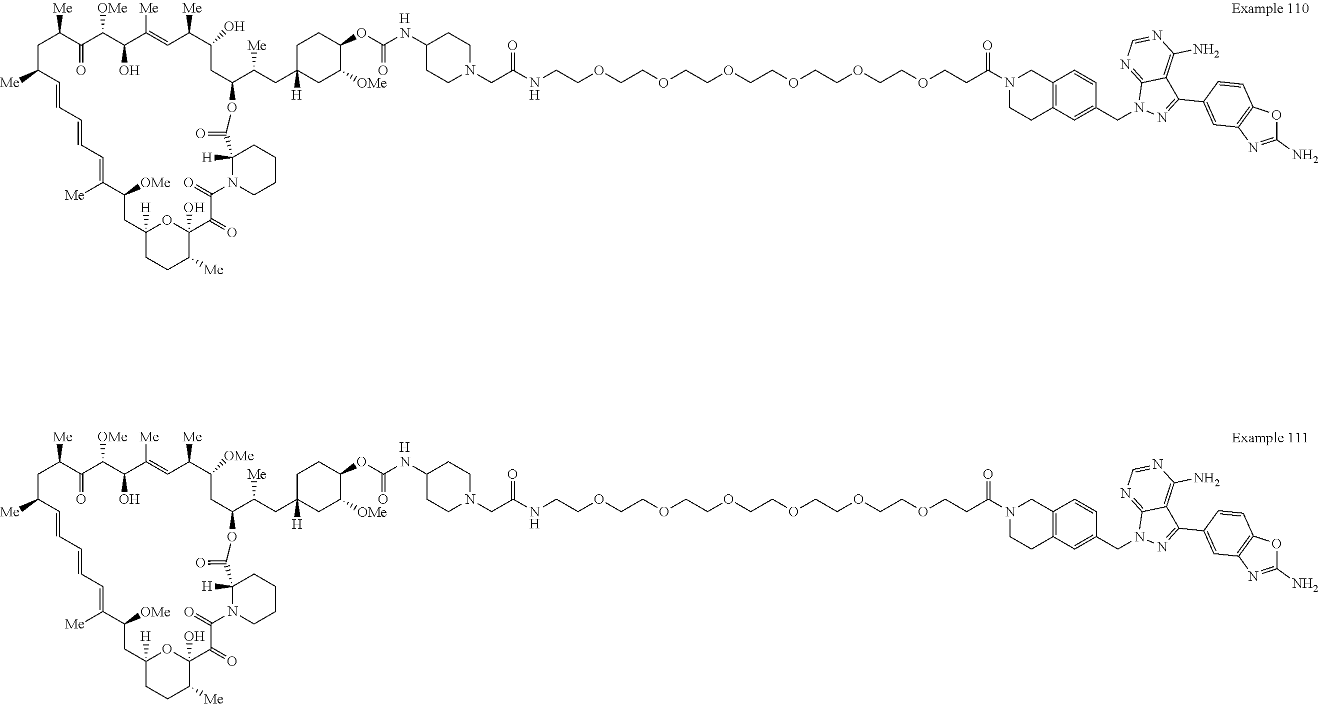

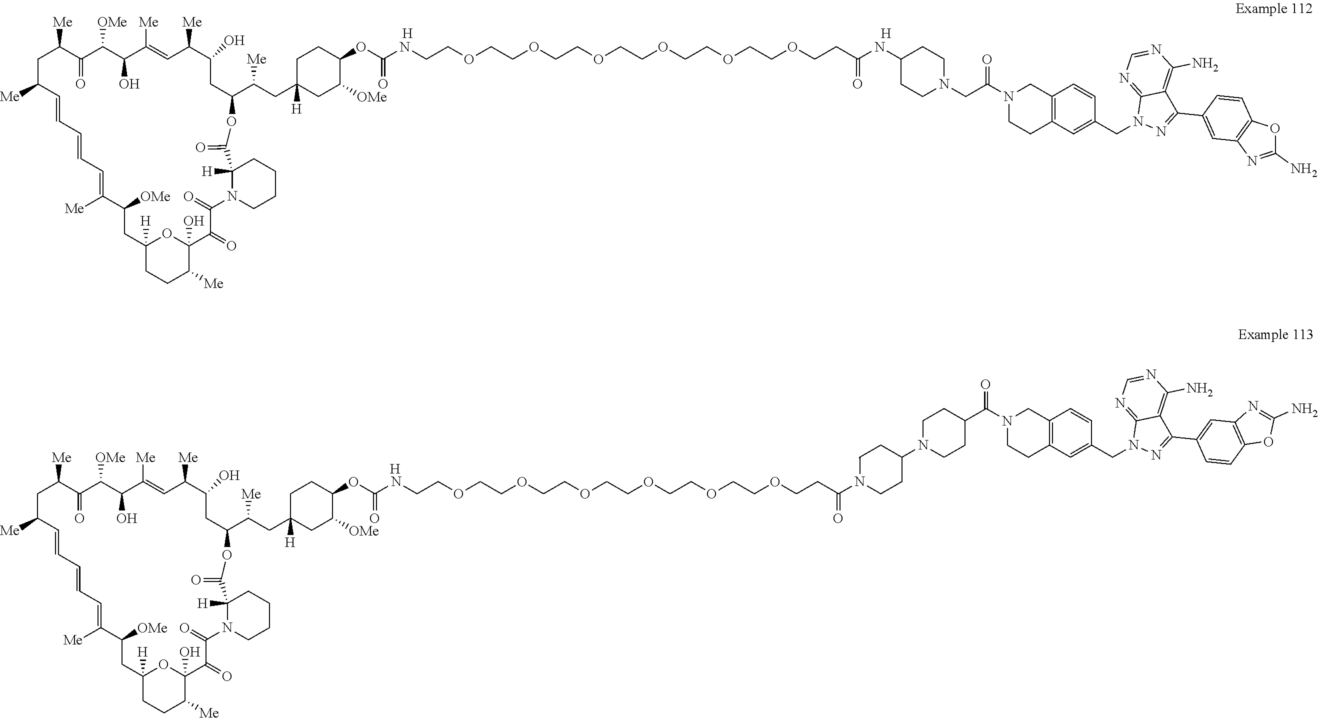

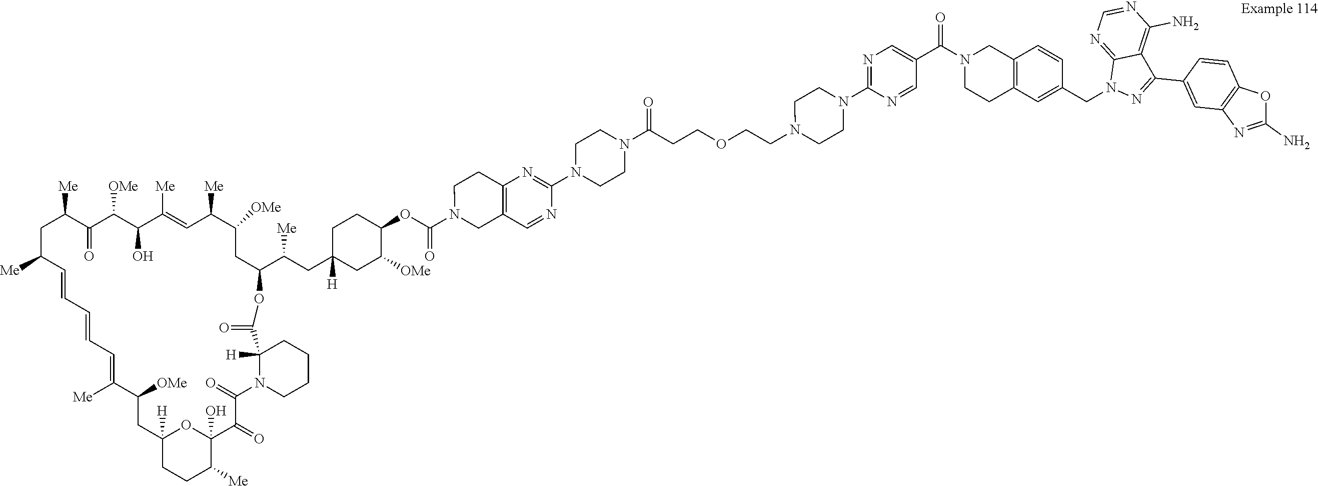 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

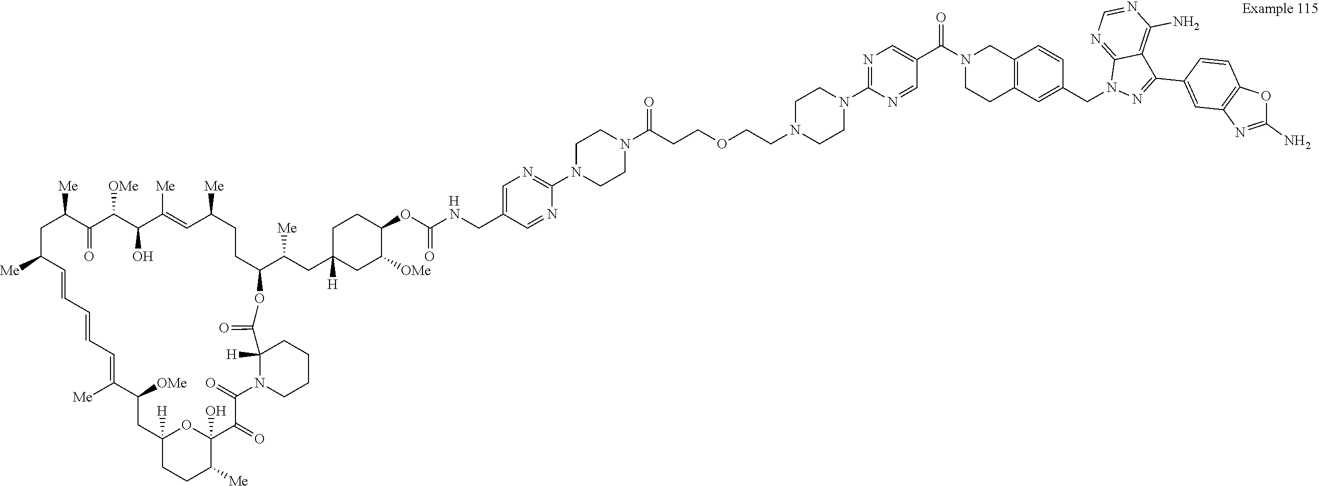

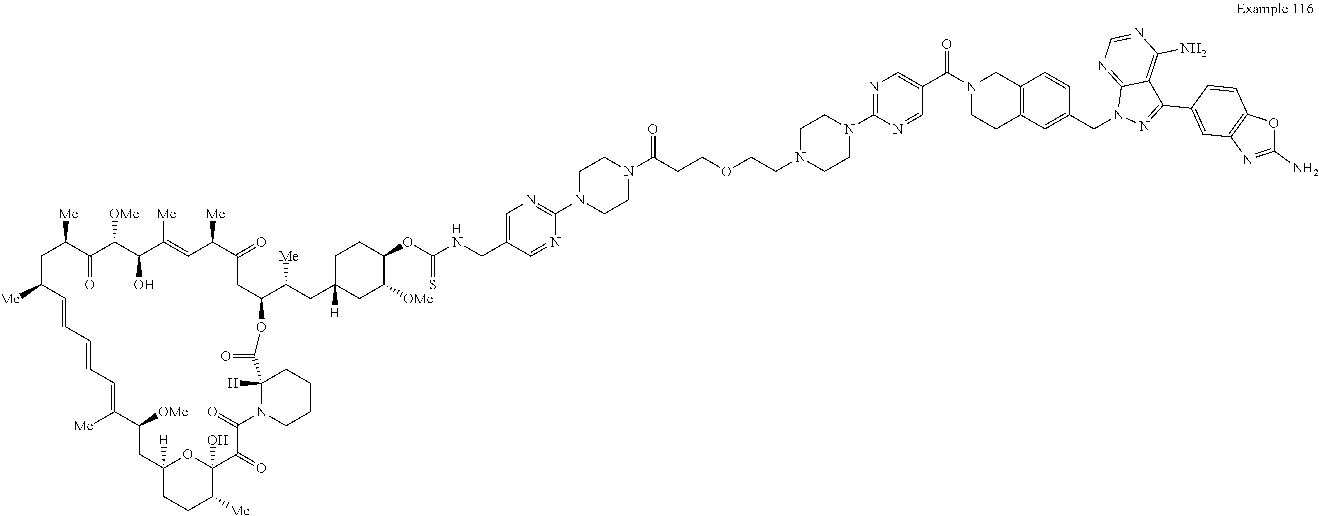

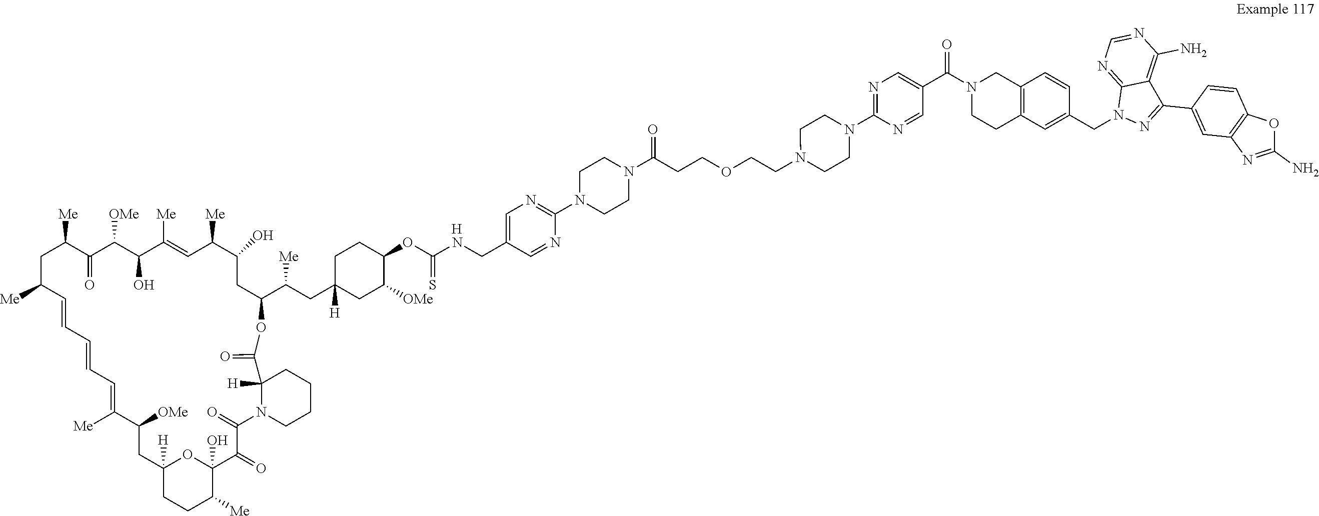

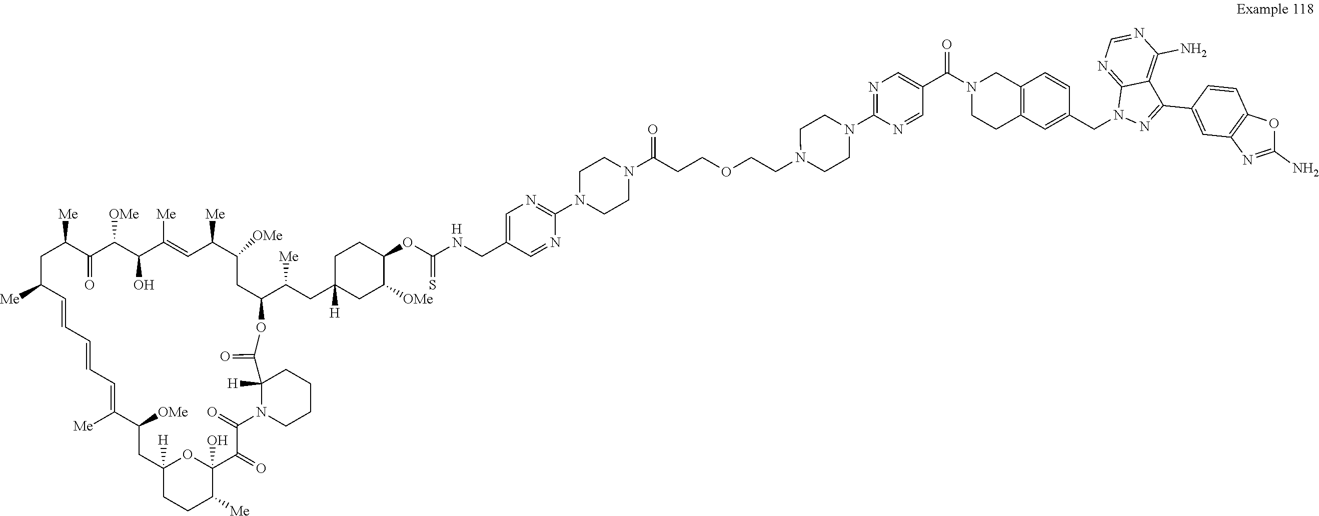

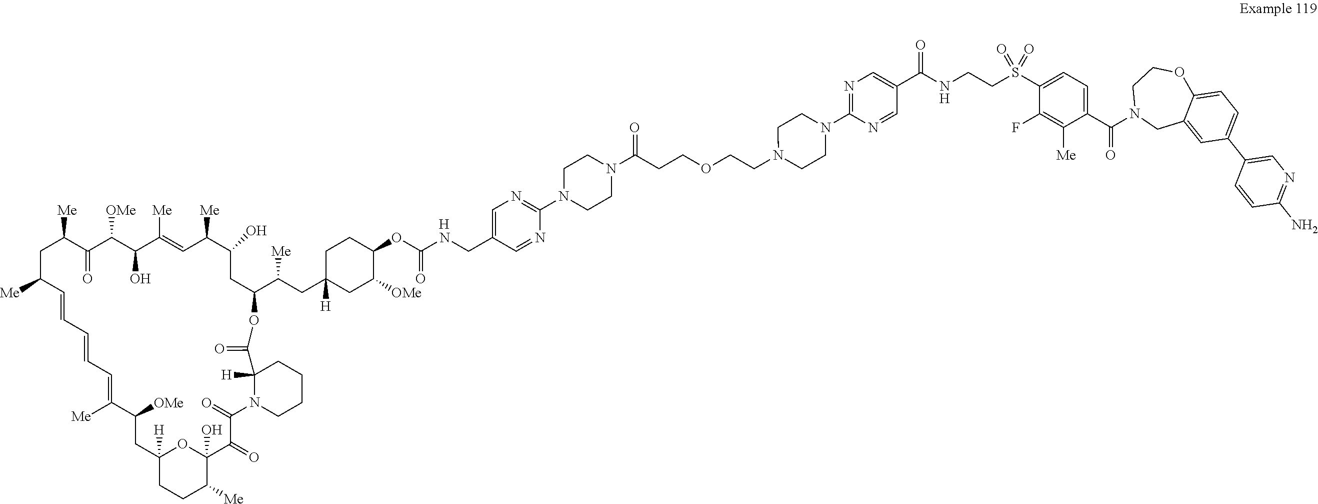

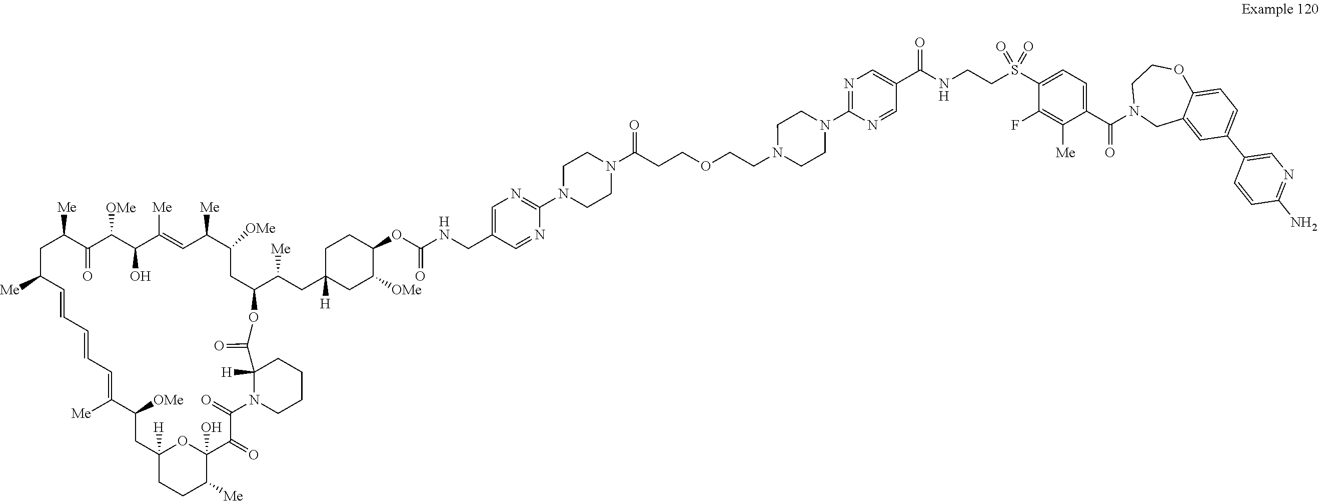

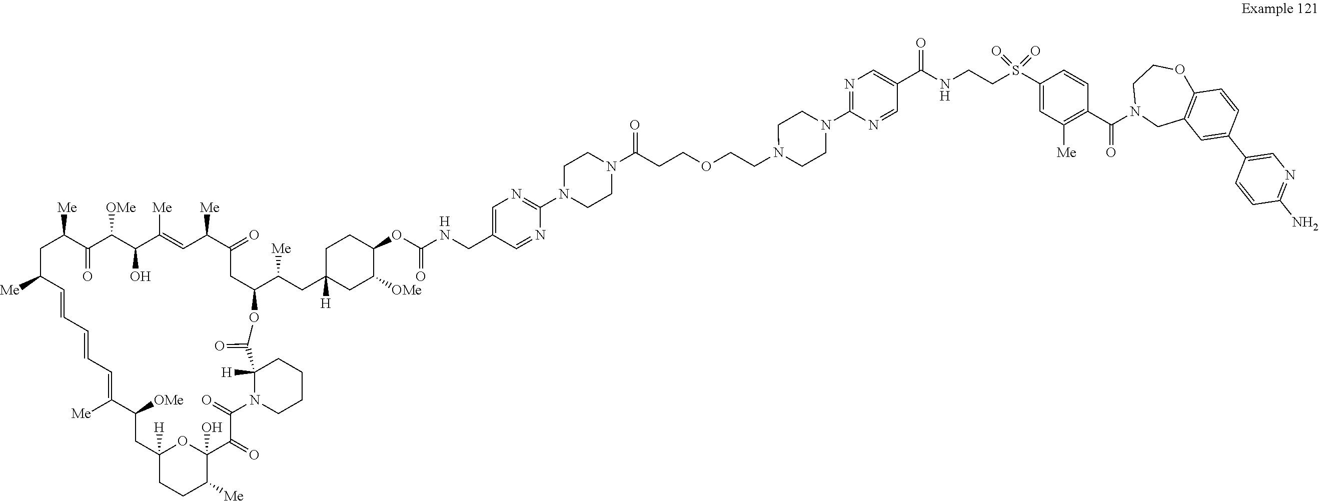

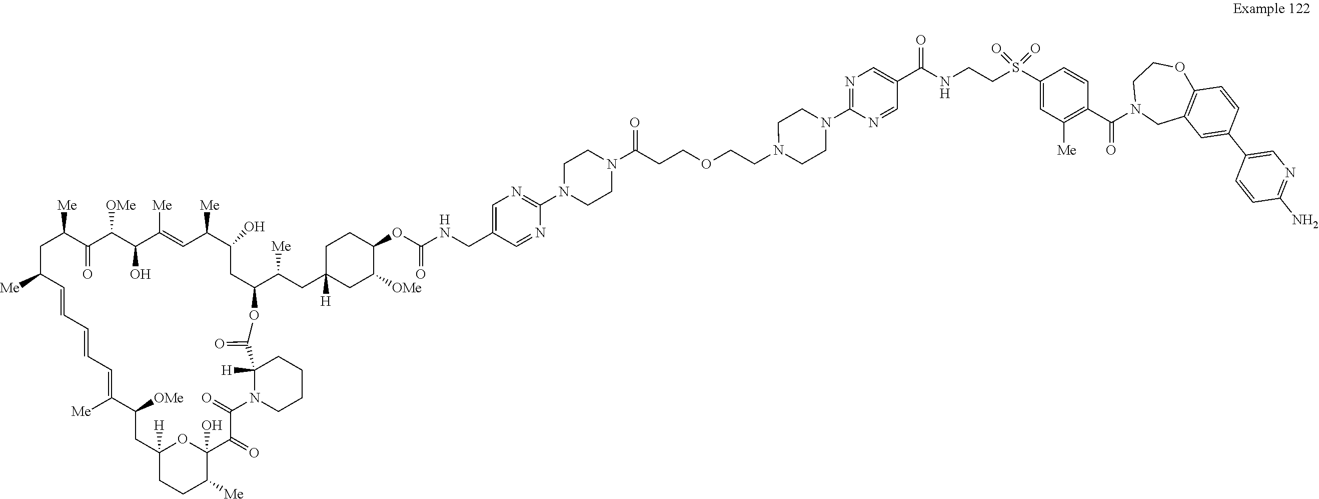

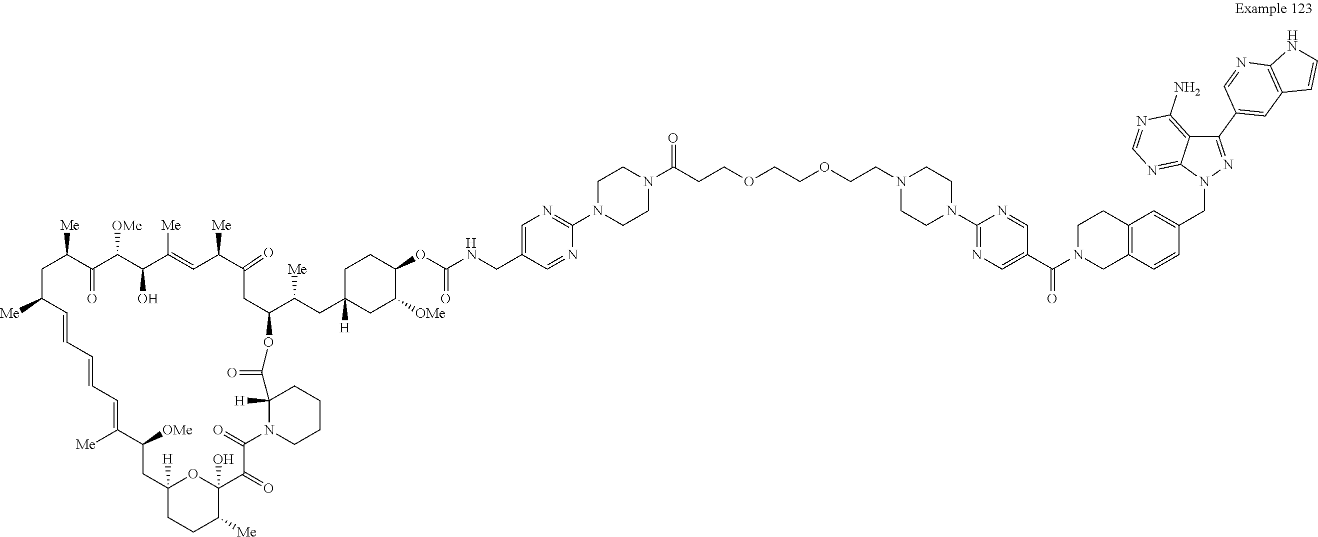

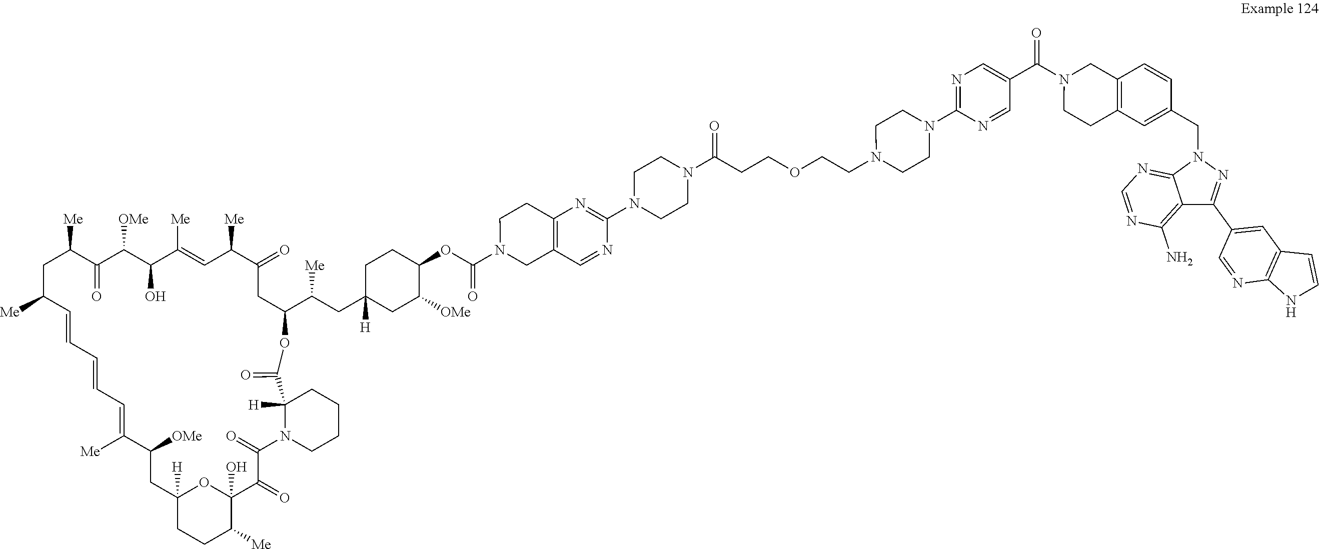 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

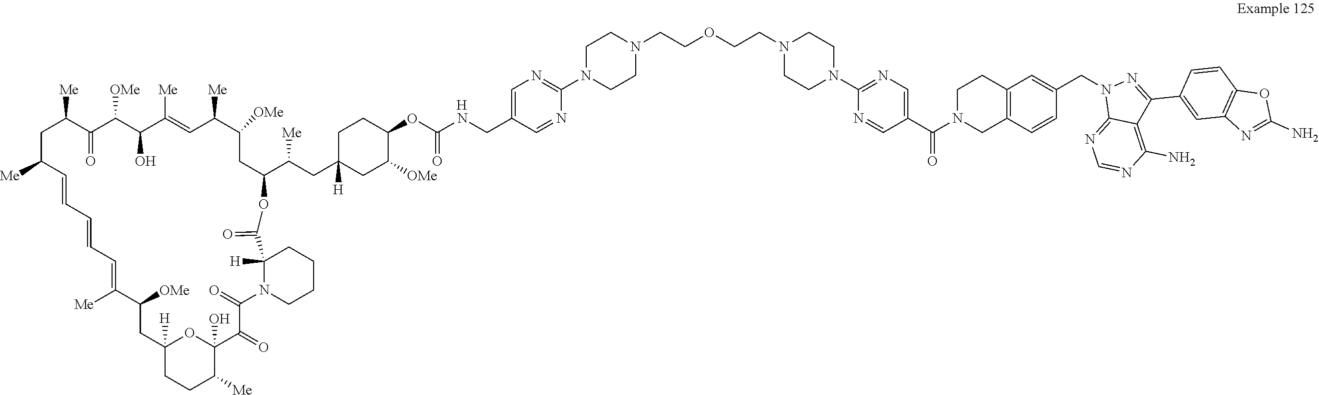

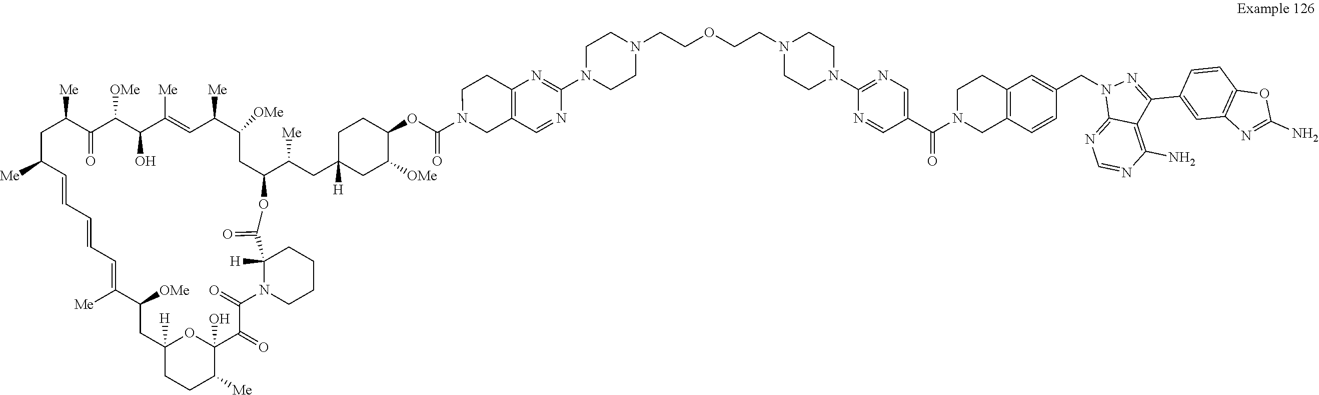 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

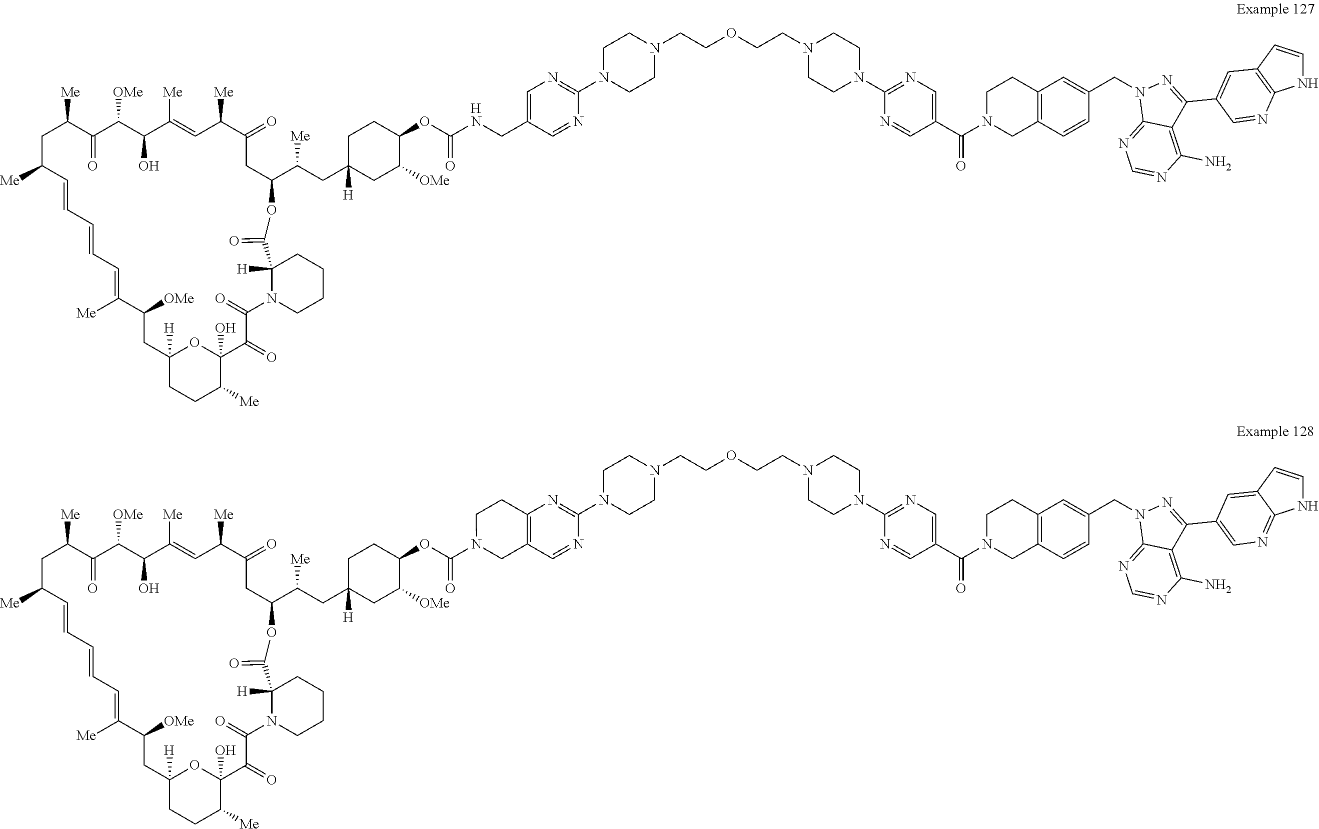

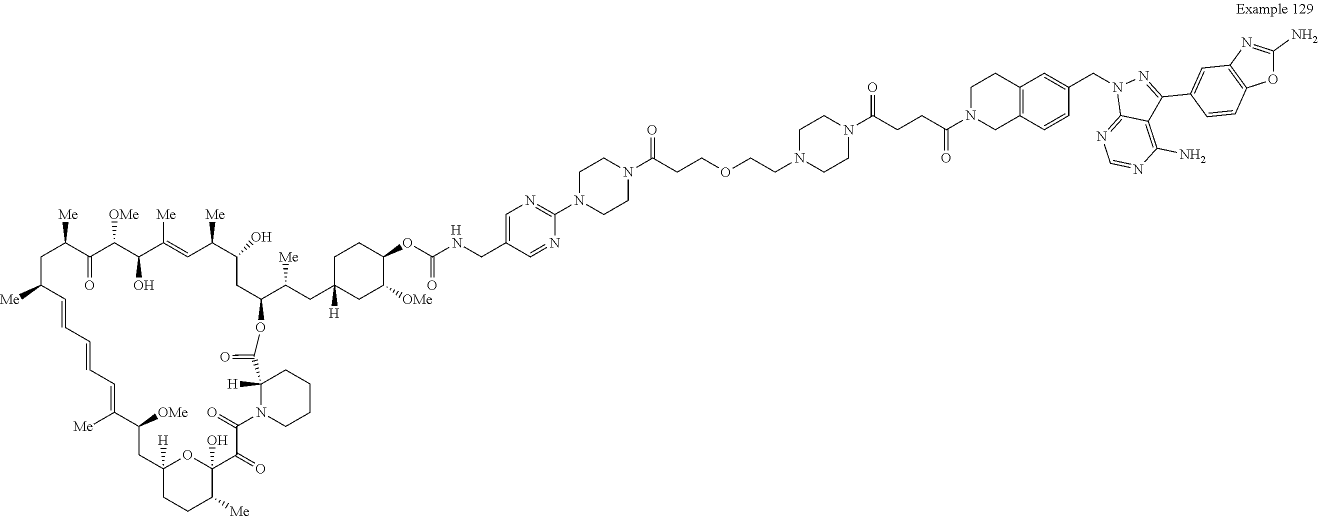

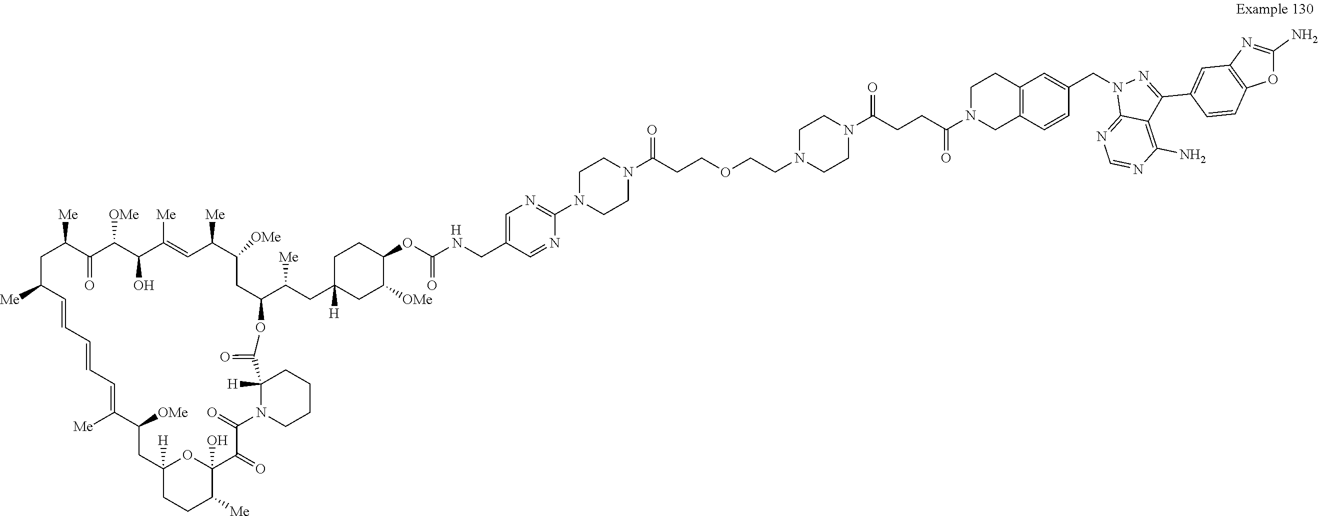

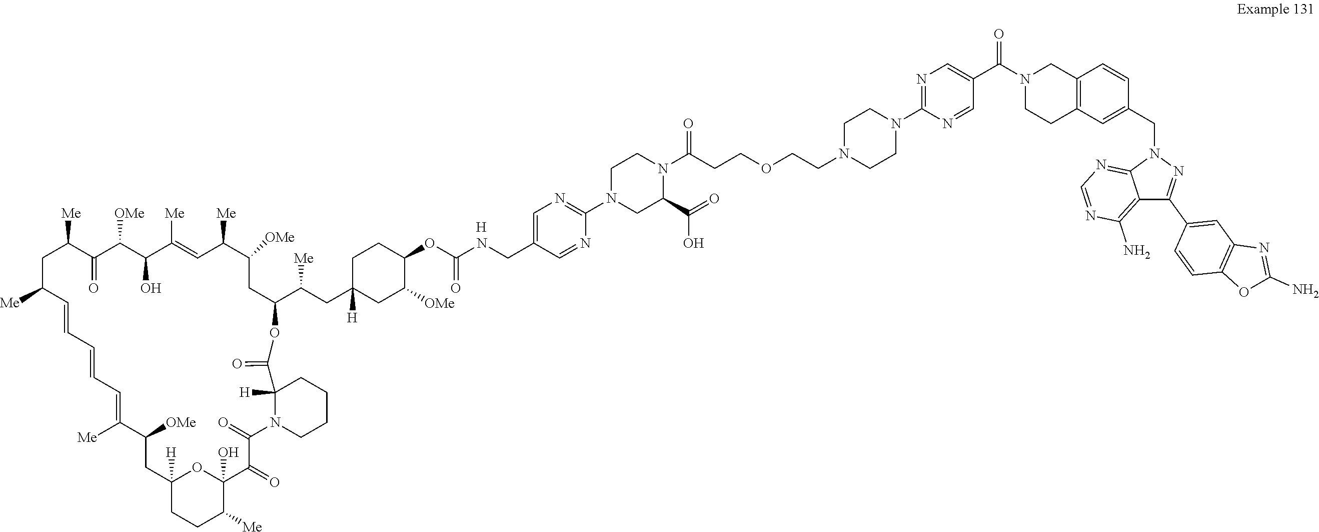

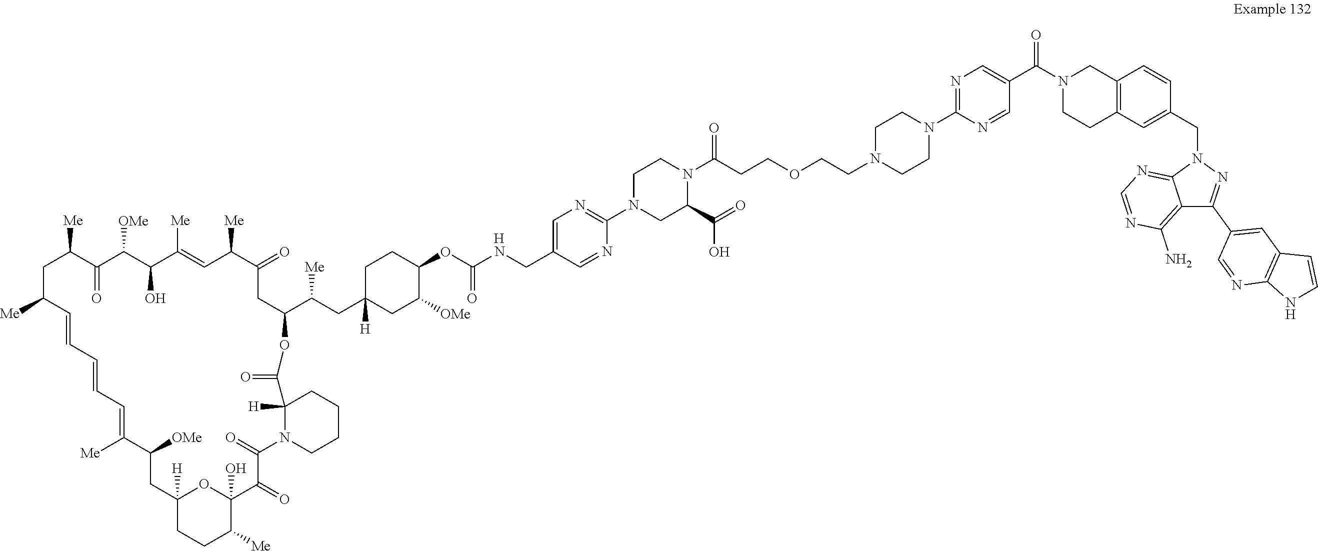

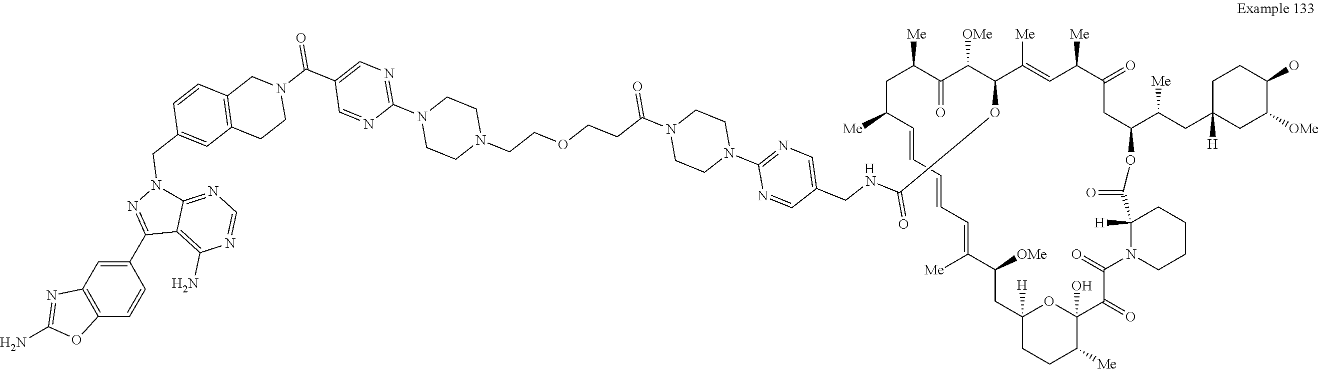

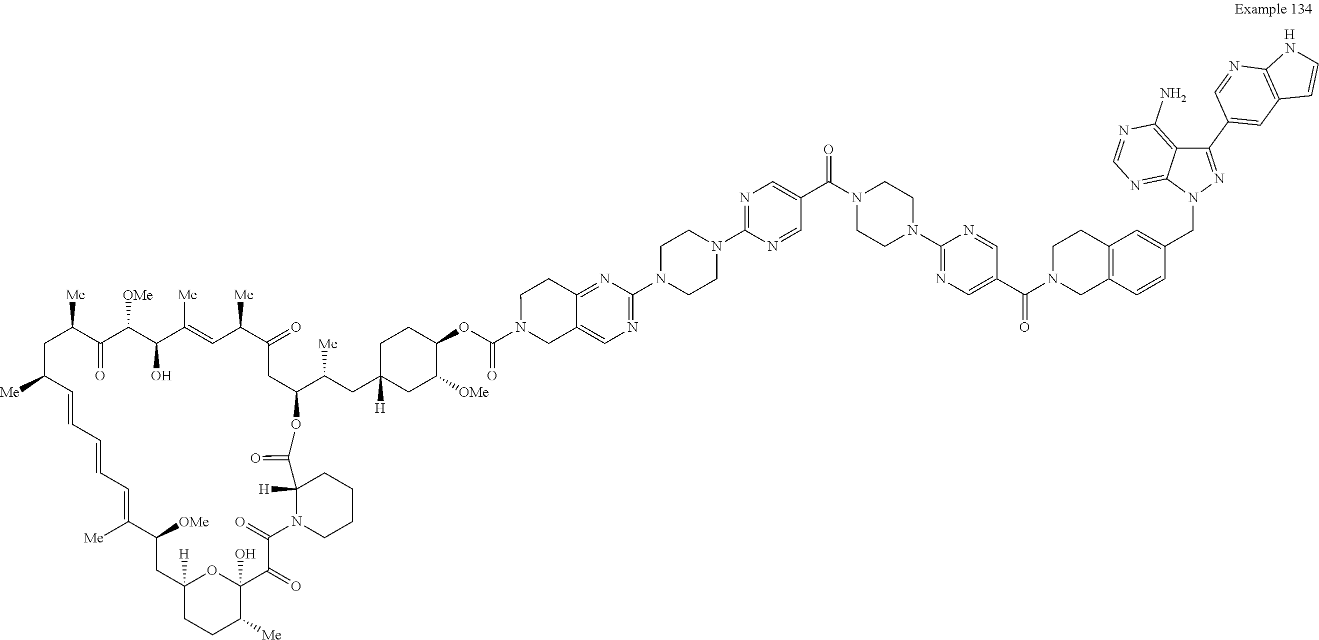

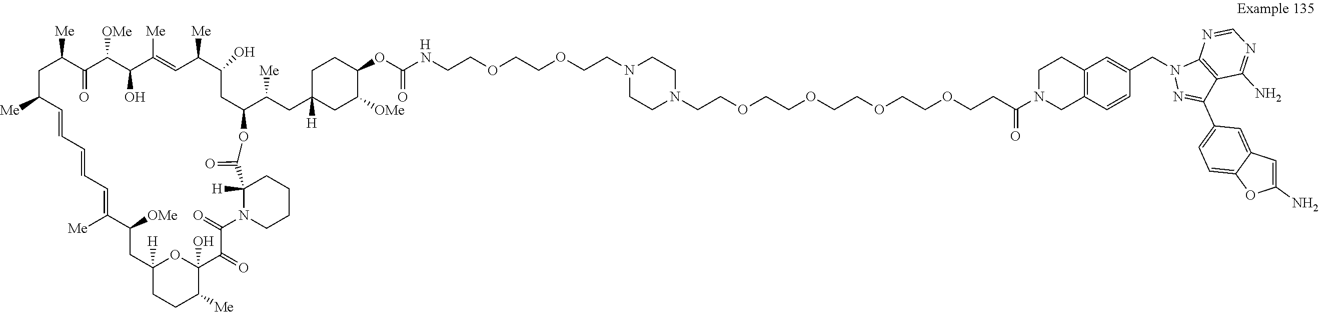

or a pharmaceutically acceptable salt or tautomer thereof.

Embodiment II-74. A compound selected from the group consisting of:

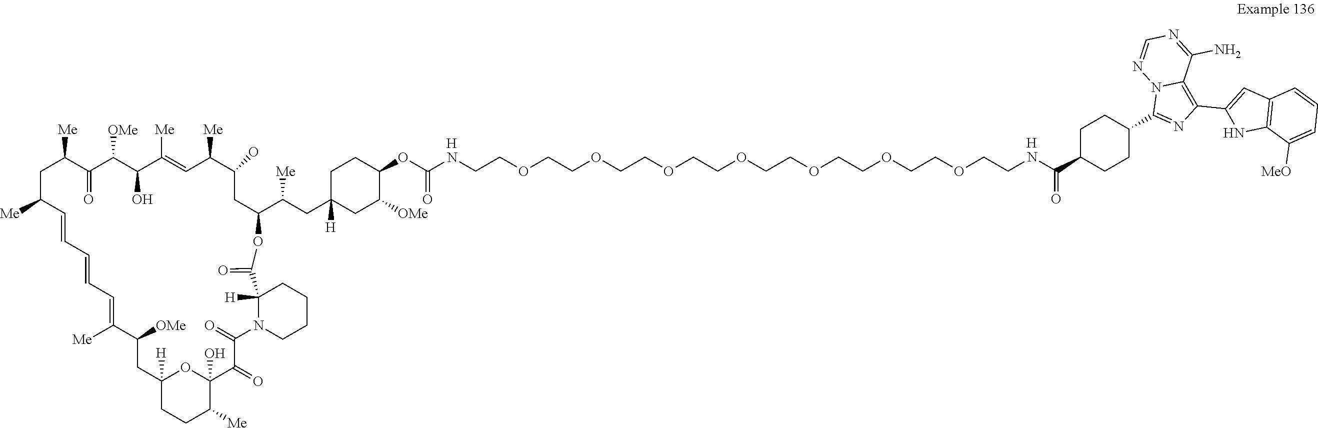

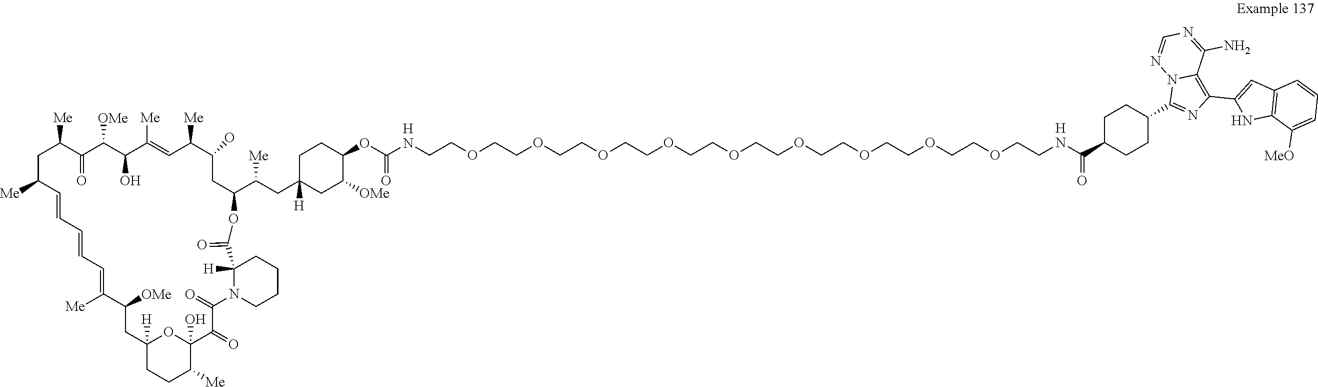

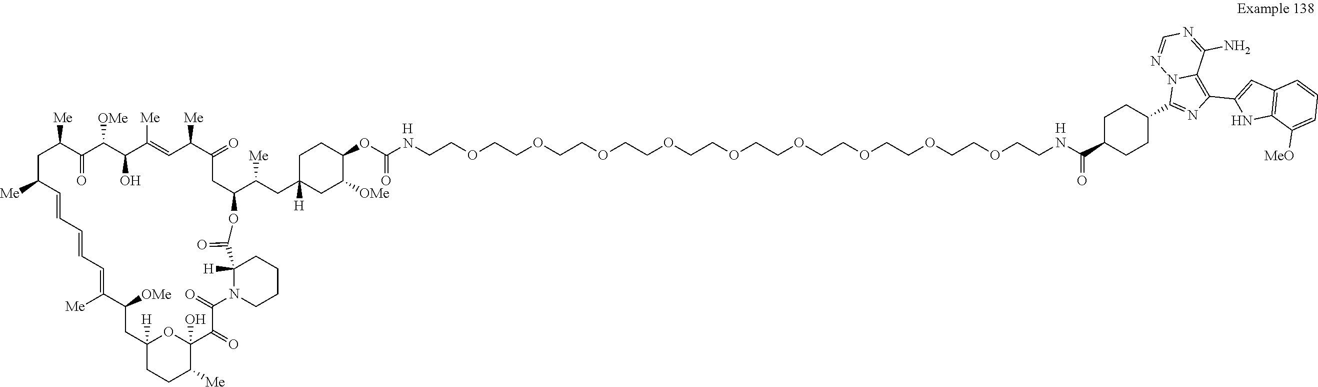

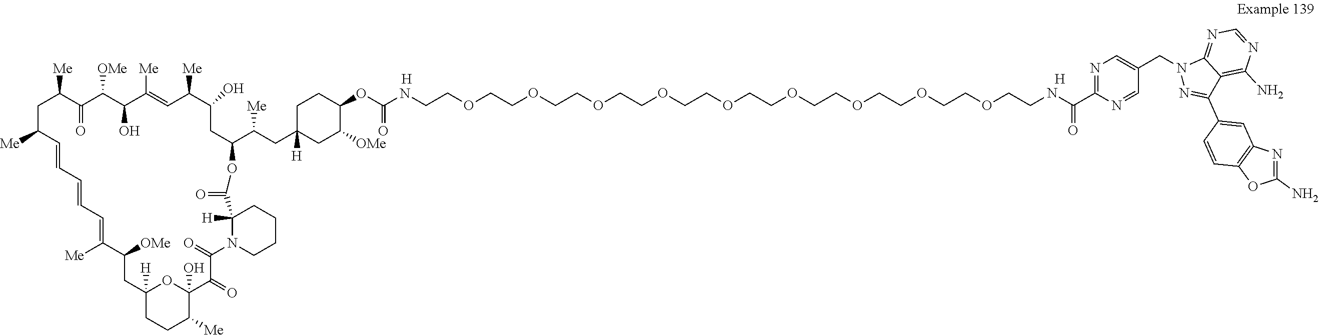

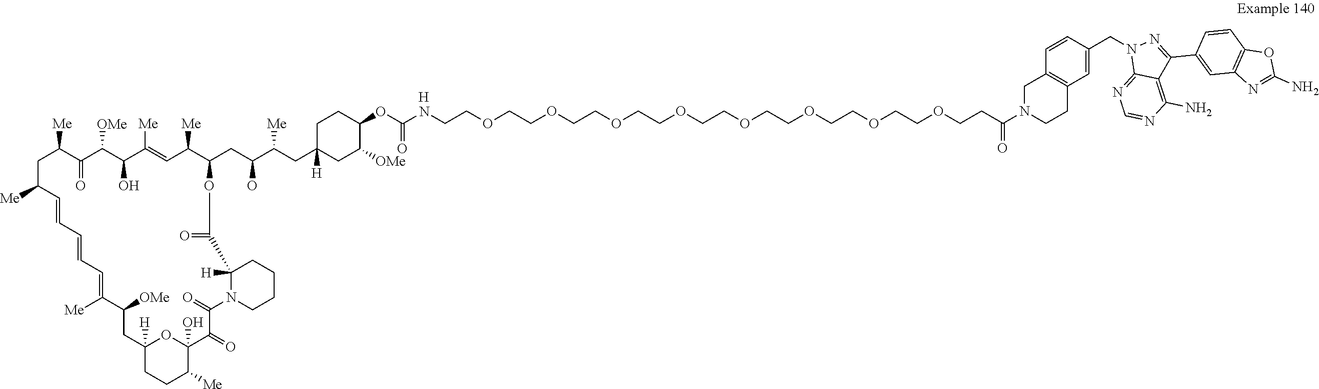

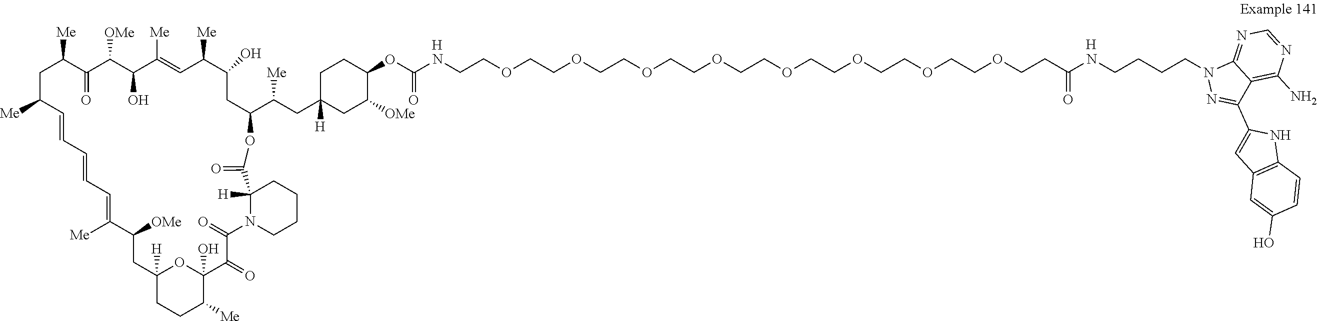

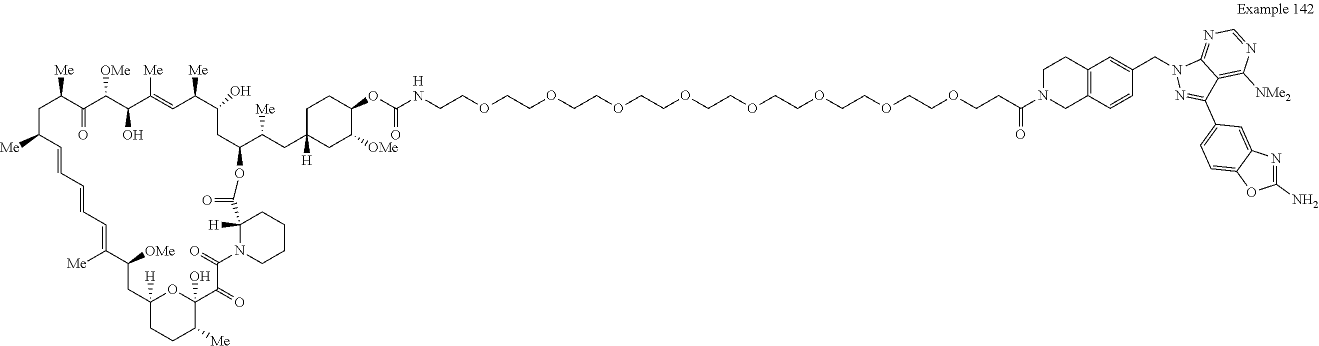

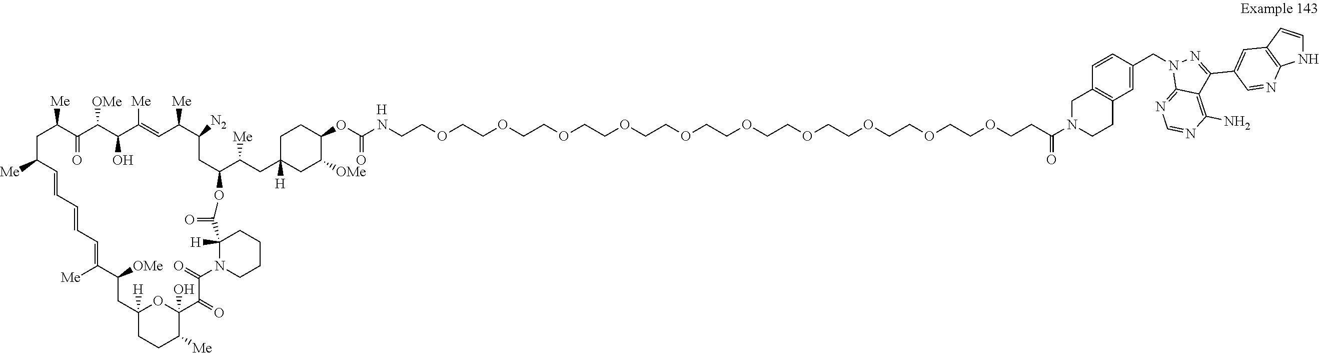

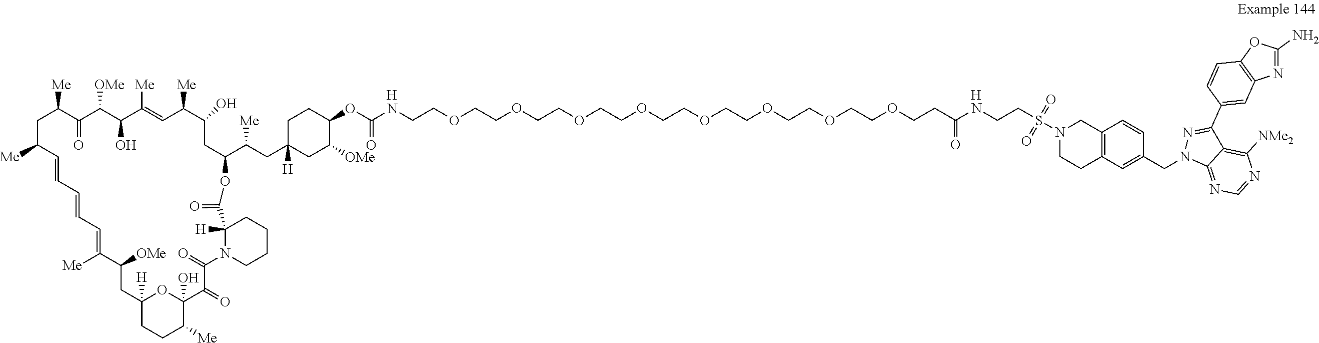

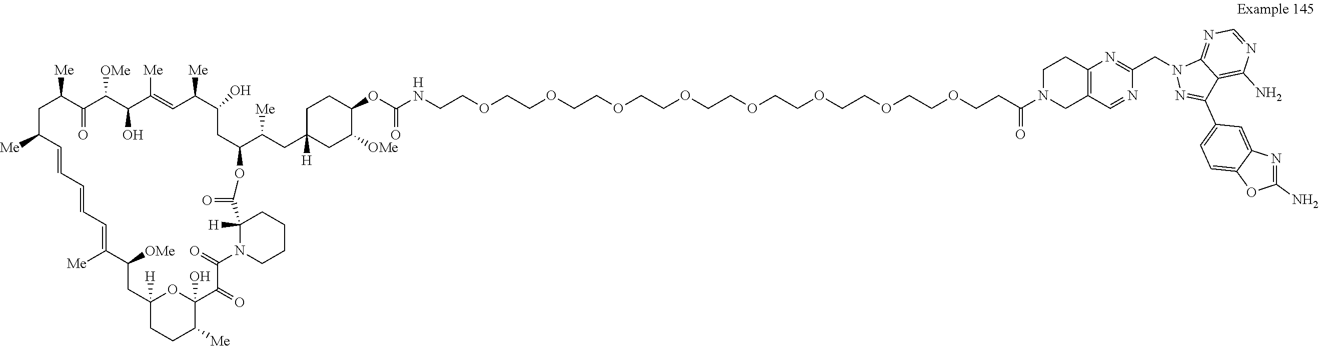

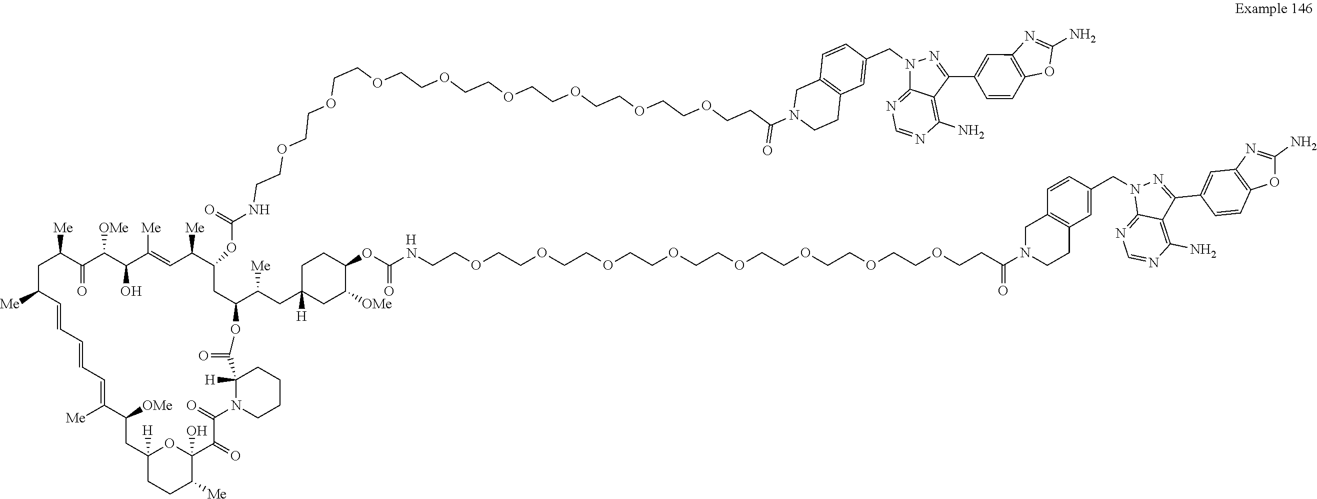

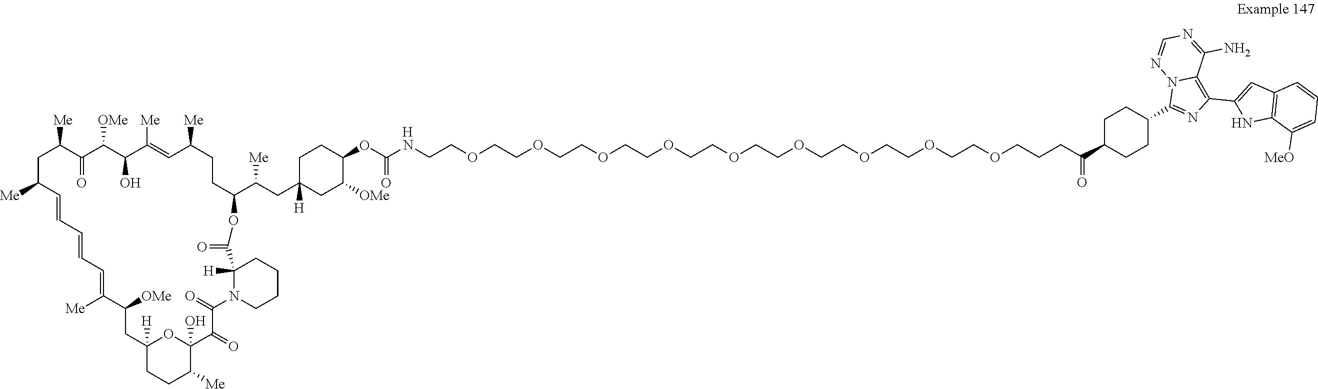

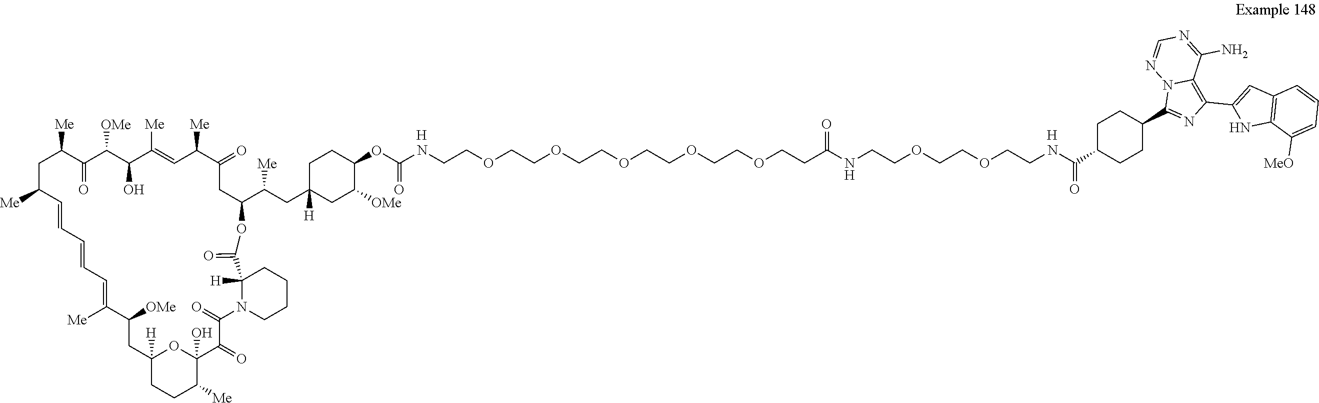

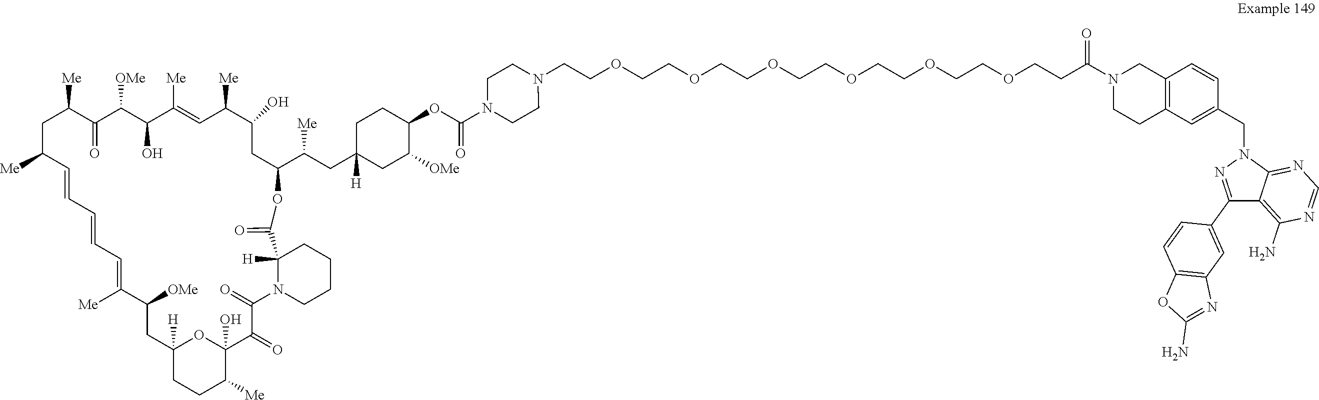 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(CCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

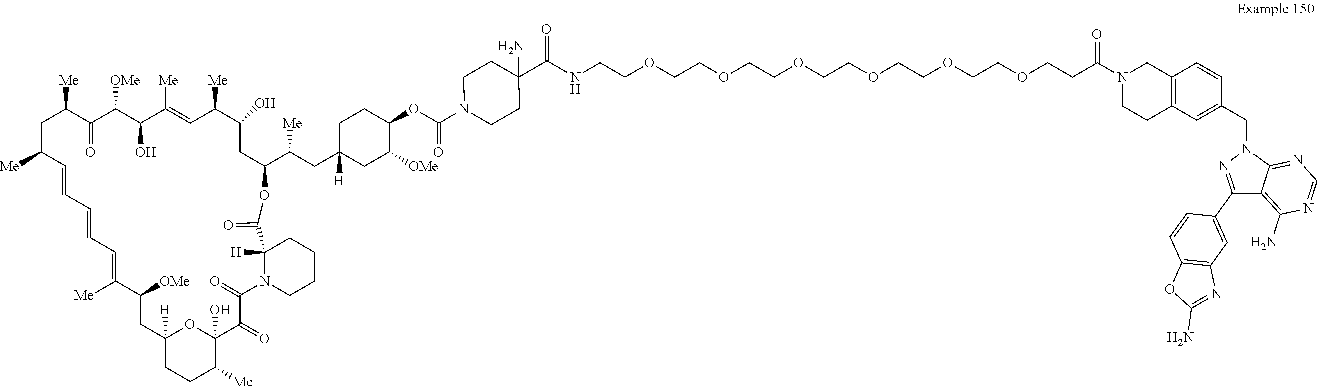 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCC(N)(C(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

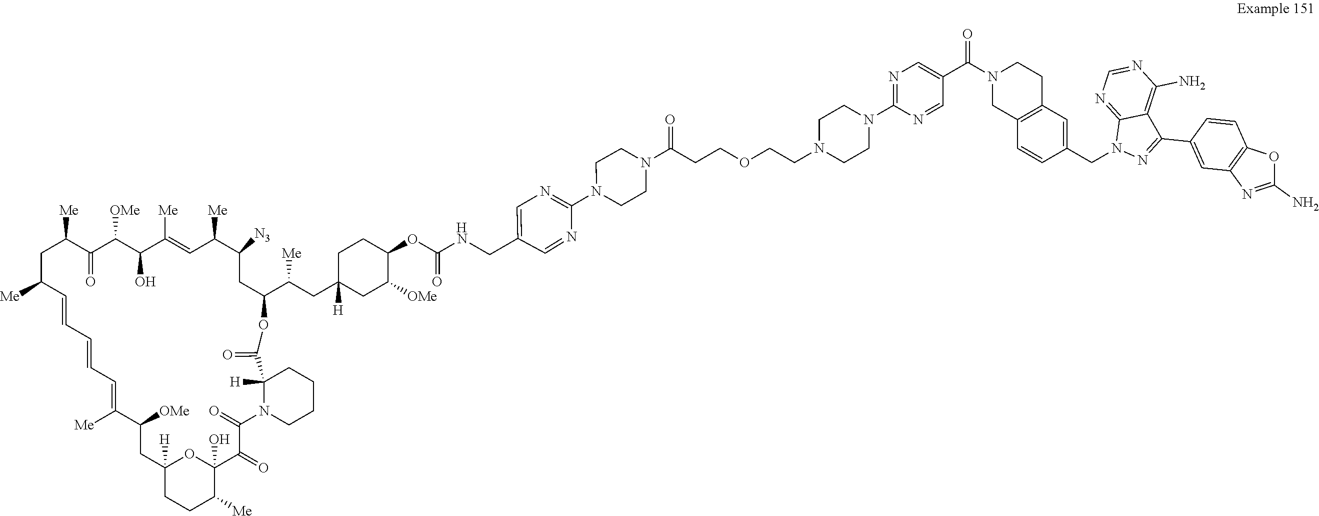

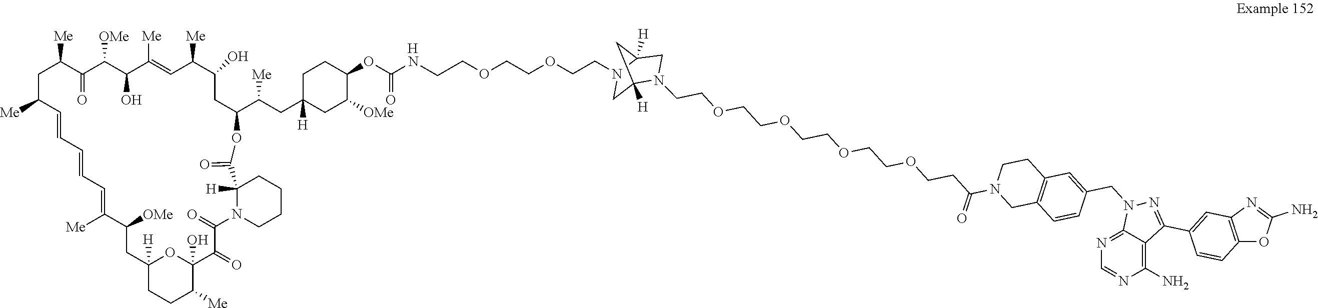

or a pharmaceutically acceptable salt or tautomer thereof.

Embodiment II-75. A pharmaceutical composition comprising a compound of any one of Embodiments II-1 to II-74, or a pharmaceutically acceptable salt thereof, and at least one of a pharmaceutically acceptable carrier, diluent, or excipient.

Embodiment II-76. A method of treating a disease or disorder mediated by mTOR comprising administering to the subject suffering from or susceptible to developing a disease or disorder mediated by mTOR a therapeutically effective amount of one or more compounds of any one of Embodiments II-1 to II-74, or a pharmaceutically acceptable salt thereof.

Embodiment II-77. A method of preventing a disease or disorder mediated by mTOR comprising administering to the subject suffering from or susceptible to developing a disease or disorder mediated by mTOR a therapeutically effective amount of one or more compounds of any one of Embodiments II-1 to II-74, or a pharmaceutically acceptable salt thereof.

Embodiment II-78. A method of reducing the risk of a disease or disorder mediated by mTOR comprising administering to the subject suffering from or susceptible to developing a disease or disorder mediated by mTOR a therapeutically effective amount of one or more compounds of any one of Embodiments II-1 to II-74, or a pharmaceutically acceptable salt thereof.

Embodiment II-79. The method of any one of Embodiments II-76 to II-78, wherein the disease is cancer or an immune-mediated disease.

Embodiment II-80. The method of Embodiment II-79, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment II-81. The method of Embodiment II-79, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment II-82. A method of treating cancer comprising administering to the subject a therapeutically effective amount of one or more compounds of any one of Embodiments II-1 to II-74, or a pharmaceutically acceptable salt thereof.

Embodiment II-83. The method of Embodiment II-82, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment II-84. A method of treating an immune-mediated disease comprising administering to the subject a therapeutically effective amount of one or more compounds of any one of Embodiments II-1 to II-74, or a pharmaceutically acceptable salt thereof.

Embodiment II-85. The method of Embodiment II-84, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment II-86. A method of treating an age related condition comprising administering to the subject a therapeutically effective amount of one or more compounds of any one of Embodiments II-1 to II-74, or a pharmaceutically acceptable salt thereof.

Embodiment II-87. The method of Embodiment II-86, wherein the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

Embodiment II-88. A compound of any one of Embodiments II-1 to II-74, or a pharmaceutically acceptable salt thereof, for use in treating, preventing, or reducing the risk of a disease or condition mediated by mTOR.

Embodiment II-89. Use of a compound of any of Embodiments II-1 to H-74, or a pharmaceutically acceptable salt thereof, in the manufacture of a medicament for treating, preventing, or reducing the risk of a disease or disorder mediated by mTOR.

Embodiment II-90. A compound of any one of Embodiments I-74, or a pharmaceutically acceptable salt thereof, for use in treating cancer.

Embodiment II-91. Use of a compound of any one of Embodiments II-1 to II-74, or a pharmaceutically acceptable salt thereof, in the manufacture of a medicament for treating cancer.

Embodiment II-92. A compound of any one of Embodiments II-1 to II-74, or a pharmaceutically acceptable salt thereof, for use in treating an immune-mediated disease.

Embodiment II-93. Use of a compound of any one of Embodiments II-1 to II-74, or a pharmaceutically acceptable salt thereof, in the manufacture of a medicament for treating an immune-mediated disease.

Embodiment II-94. A compound of any one of Embodiments II-1 to II-74, or a pharmaceutically acceptable salt thereof, for use in treating an age related condition.

Embodiment II-95. Use of a compound of any one of Embodiments II-1 to II-74, or a pharmaceutically acceptable salt thereof, in the manufacture of a medicament for treating an age related condition.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the claims.

Definitions used in the following examples and elsewhere herein are:

| | |
|---|---|
| CH₂Cl₂, DCM | Methylene chloride, Dichloromethane |
| CH₃CN, MeCN | Acetonitrile |
| DIPEA | Diisopropylethyl amine or Hunig's base |
| DMA | Dimethylacetamide |
| DME | Dimethoxyethane |
| DMF | N,N-Dimethylformamide |
| EDCI | 1-Ethyl-3-(3-dimethylaminopropyl)carbodiimide |
| EtOAc | Ethyl acetate |
| h | hour |
| H₂O | Water |
| HCl | Hydrochloric acid |
| HOBt | Hydroxybenzotriazole |
| HPLC | High-performance liquid chromatography |
| LCMS | Liquid chromatorgraphy-mass spectrometry |
| MeOH | Methanol |
| MTBE | Methyl tert-butyl ether |
| Na₂SO₄ | Sodium sulfate |
| PEG | Polyethylene glycol |
| g | tert-butyldimethylsilyl |
| TFA | Trifluoroacetic acid |
| THF | Tetrahydrofuran |
| TMS | Tetramethylsilane |

### Series 1 Bifunctional Rapalogs

A general structure of Series 1 bifunctional rapalogs is shown in Scheme 1 below. For these types of bifunctional rapalogs, the linker may include variations where q=0 to 30, such as q=1 to 7, and r=1 to 6. The linker amine can include substitutions, such as R═H and C1-C6 alkyl groups. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

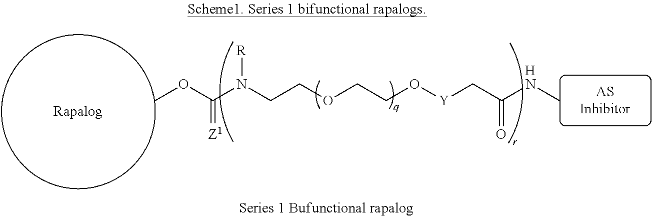

### Series 2 Bifunctional Rapalogs

A general structure of Series 2 bifunctional rapalogs is shown in Scheme 2 below. For these types of bifunctional rapalogs, the linker may include variations where q=0 to 30, such as q=1 to 7. The linker amine can include substitutions, such as R═H and C1-C6 alkyl groups. The pre-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

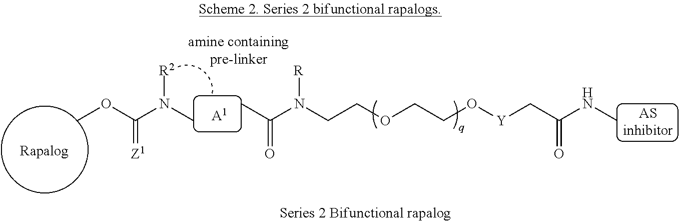

### Series 3 Bifunctional Rapalogs

A general structure of Series 3 bifunctional rapalogs is shown in Scheme 3 below. For these types of bifunctional rapalogs, the linker may include variations where q=0 to 30, such as q=1 to 7. The linker amine can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

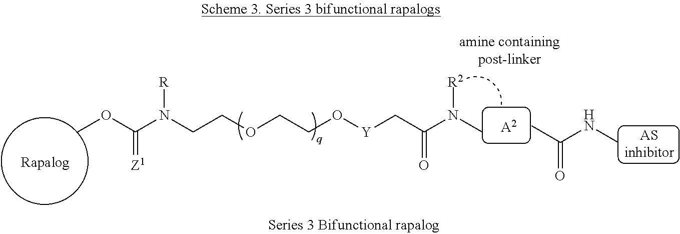

### Series 4 Bifunctional Rapalogs

A general structure of Series 4 bifunctional rapalogs is shown in Scheme 4 below. For these types of bifunctional rapalogs, the linker may include variations where q=0 to 30, such as q=1 to 7. The linker amine can include substitutions, such as R═H and C1-C6 alkyl groups. The pre- and post-linker amines can each include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

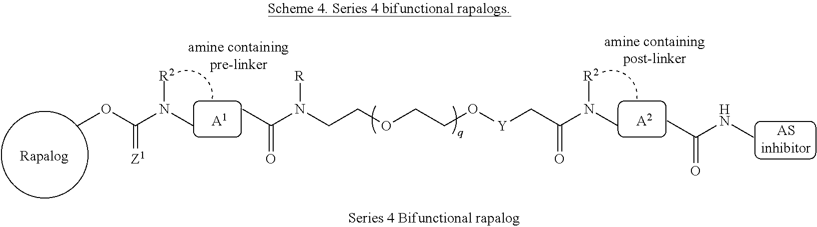

### Series 5 Bifunctional Rapalogs

A general structure of Series 5 bifunctional rapalogs is shown in Scheme 5 below. For these types of bifunctional rapalogs, the pre-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

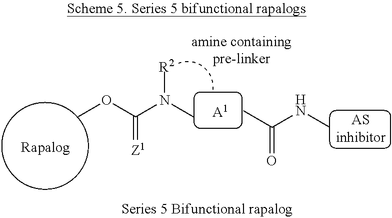

### Series 6 Bifunctional Rapalogs

A general structure of Series 6 bifunctional rapalogs is shown in Scheme 6 below. For these types of bifunctional rapalogs, the linker may include variations where q=0 to 30, such as q=1 to 7. The linker amines can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

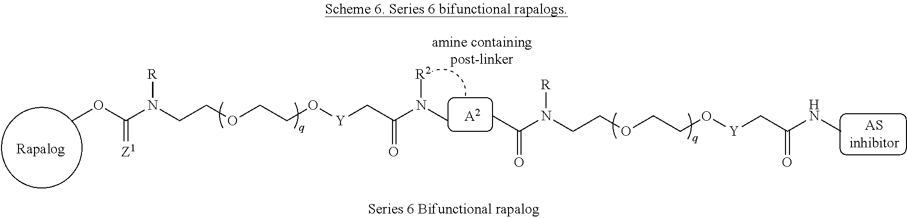

### Series 7 Bifunctional Rapalogs

A general structure of Series 7 bifunctional rapalogs is shown in Scheme 7 below. For these types of bifunctional rapalogs, the linker may include variations where q=0 to 30, such as q=1 to 7. The linker amine can include substitutions, such as R═H and C1-C6 alkyl groups. The pre- and post-linker amines can each include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I or II), including variations found in Table 1 in the Examples Section. An mTOR active site inhibitor can attach to the linker via a primary or secondary amine, and may include variations found in Table 2 in the Examples Section.

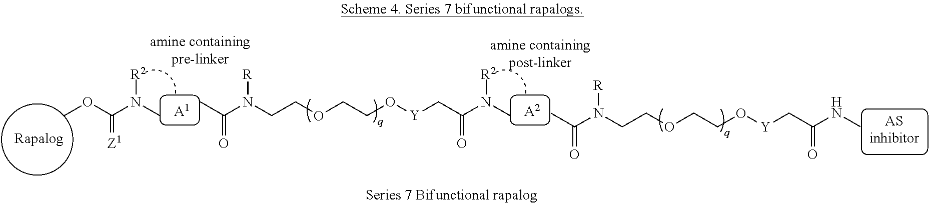

### Series 8 Bifunctional Rapalogs

A general structure of Series 8 bifunctional rapalogs is shown in Scheme 8 below. For these types of bifunctional rapalogs, the linker may include variations where q=0 to 30, such as q=1 to 7. The linker amine can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I or II), including variations found in Table 1 in the Examples Section. An mTOR active site inhibitor can attach to the linker via a primary or secondary amine, and may include variations found in Table 2 in the Examples Section.

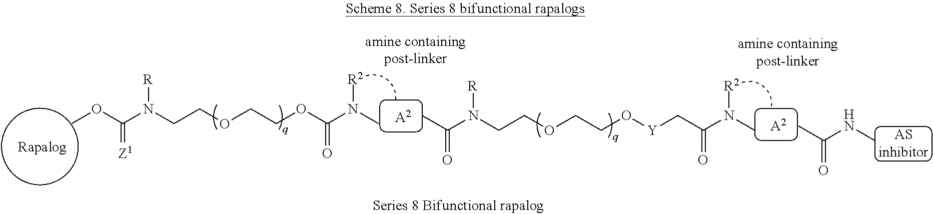

TABLE 1
Carbonate and thiocarbonate containing rapalog monomers.
Carbonate containing rapalog

| | |
|---|---|
| 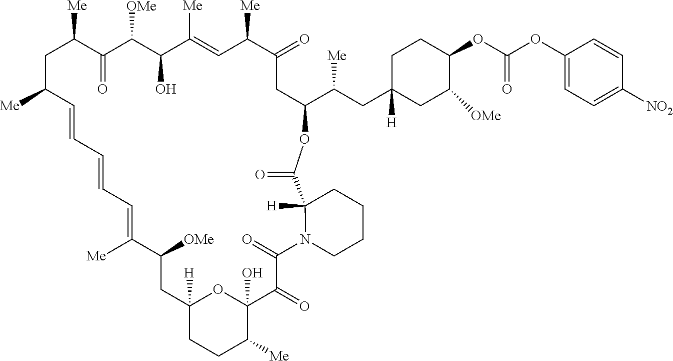 | Monomer 1 |
| 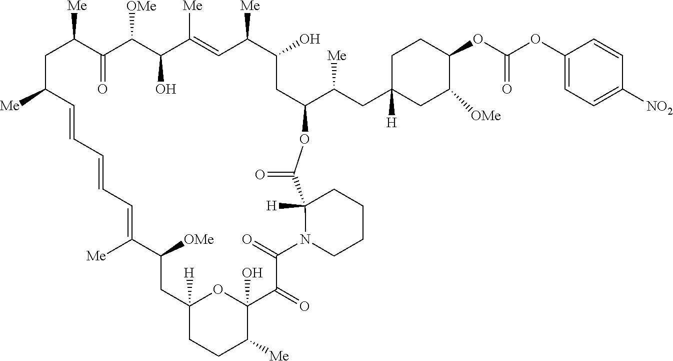 | Monomer 2 |
| 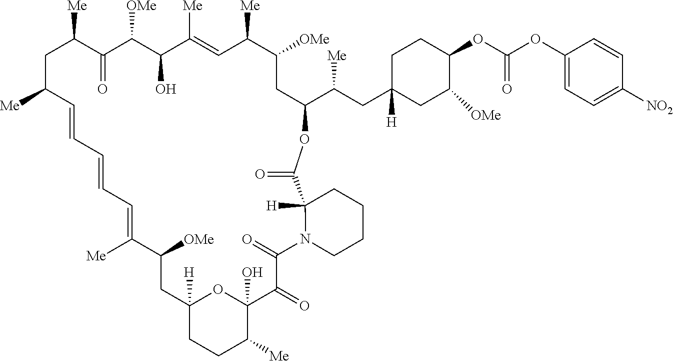 | Monomer 3 |
| 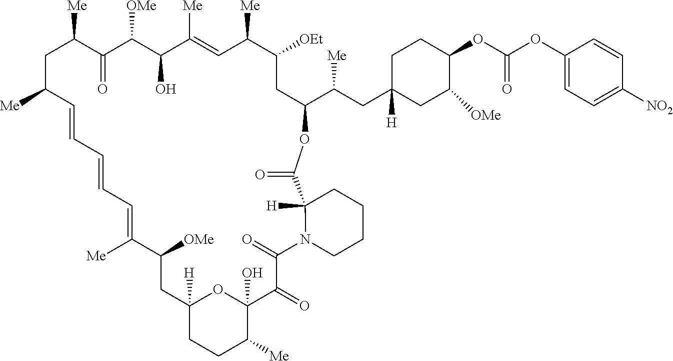 | Monomer 4 |
| 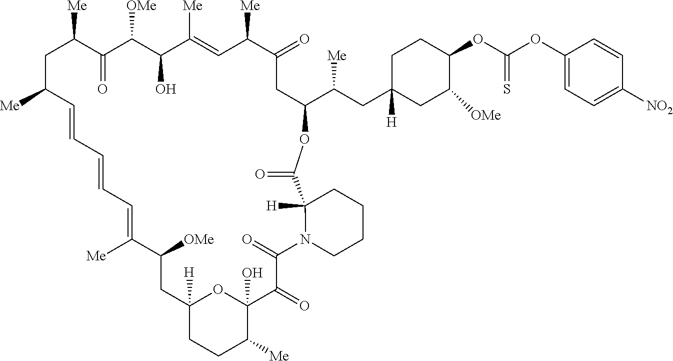 | Monomer 5 |
| 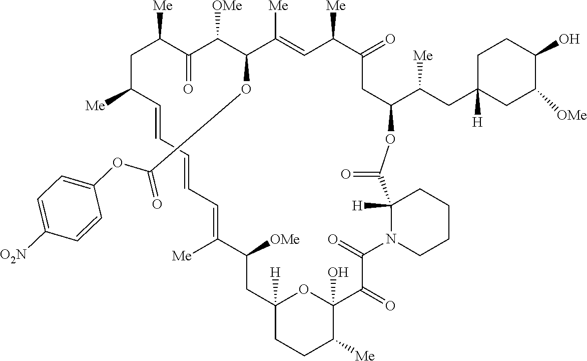 | Monomer 6 |
| 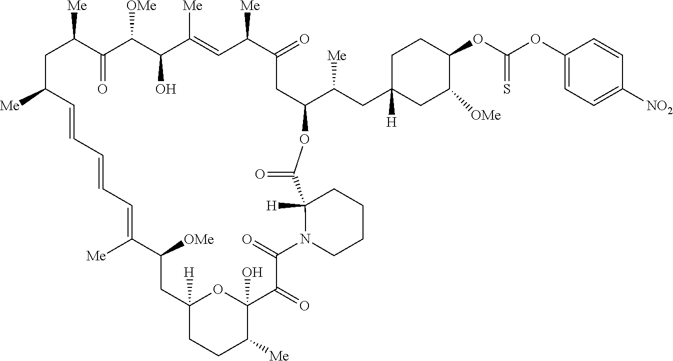 | Monomer 7 |
| 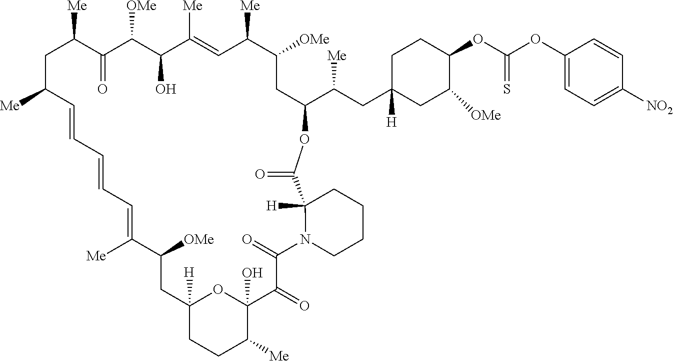 | Monomer 8 |
| 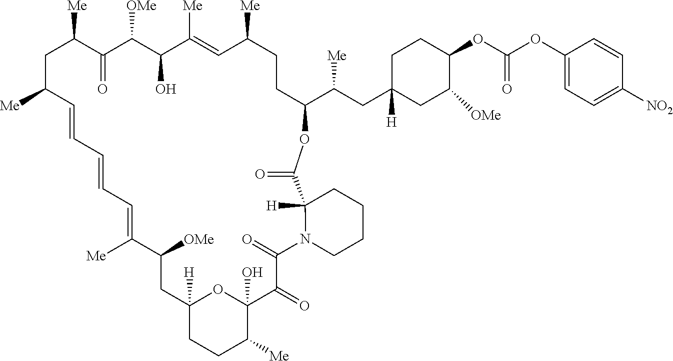 | Monomer 9 |
| 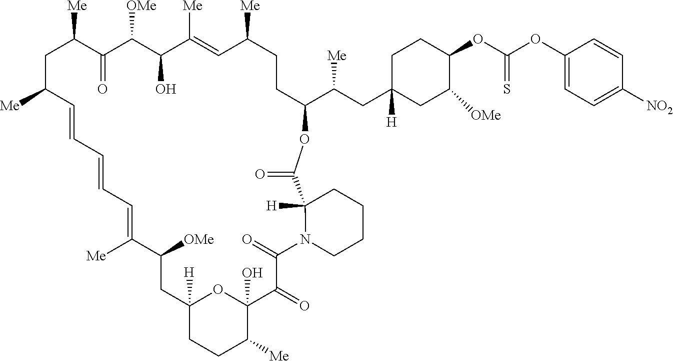 | Monomer 10 |
| 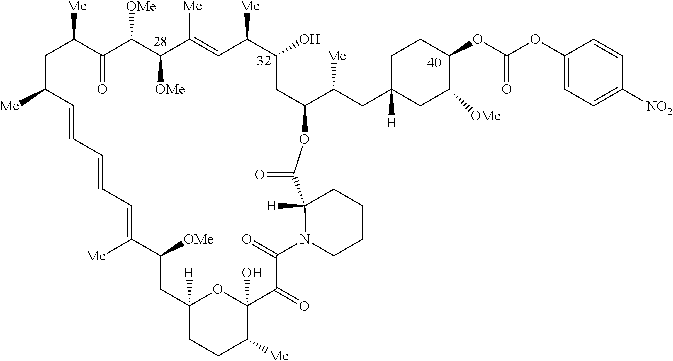 | Monomer 11 |
| 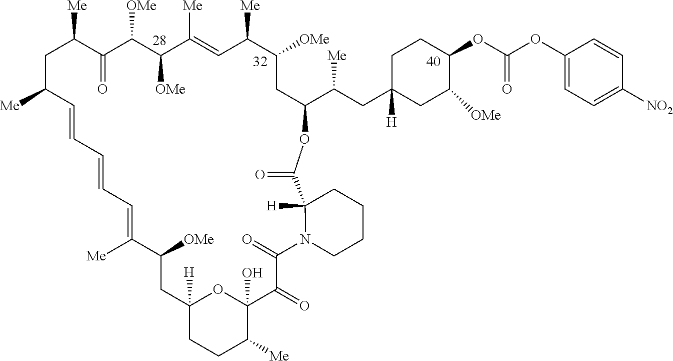 | Monomer 12 |
| 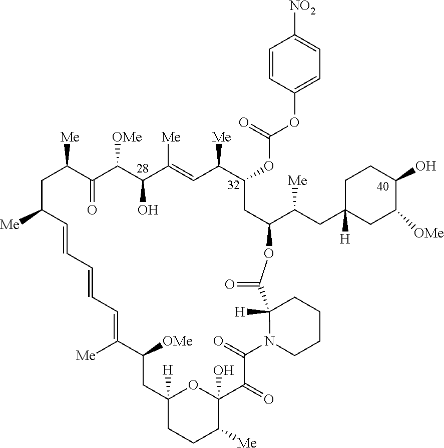 | Monomer 13 |
| 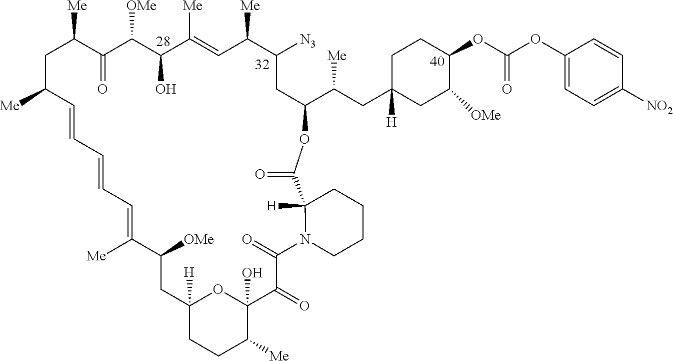 | Monomer 14 |
| 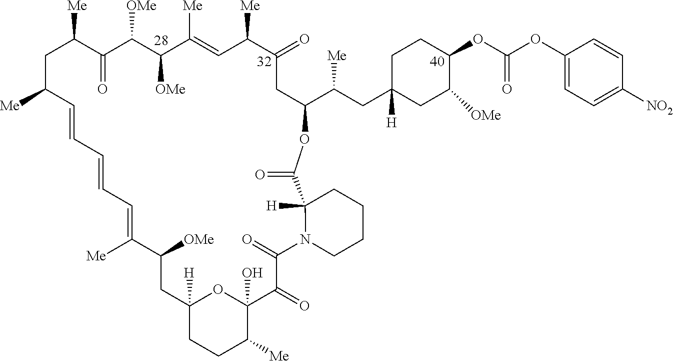 | Monomer 15 |
| 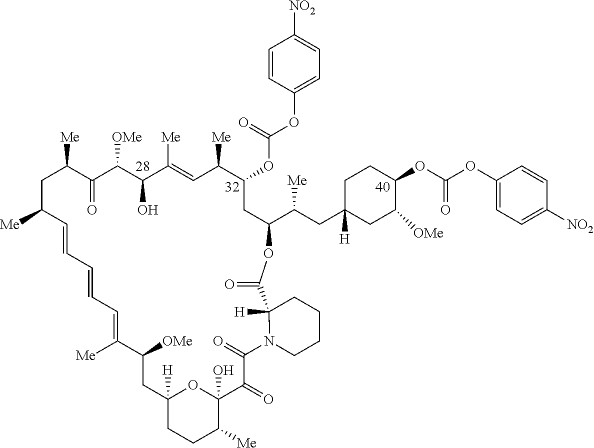 | Monomer 16 |
| 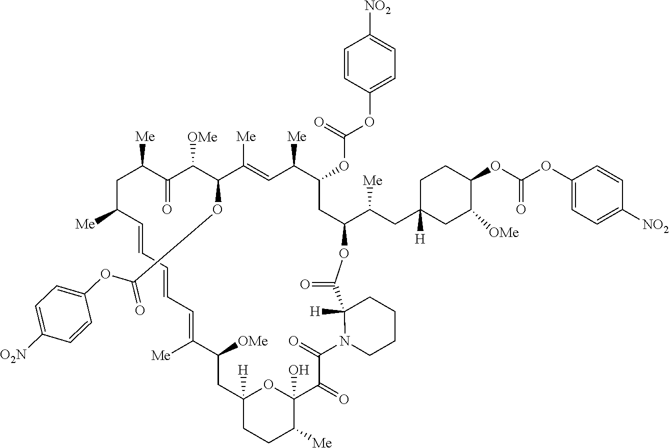 | Monomer 17 |

TABLE 2
Active Site inhibitor monomers.
Active Site inhibitor monomers

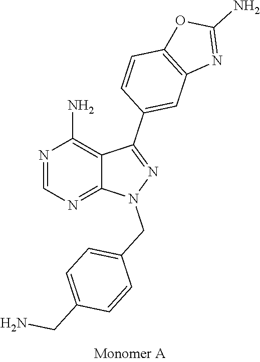 [SMILES: NCc1ccc(Cn2nc(-c3ccc4oc(N)nc4c3)c3c(N)ncnc32)cc1]

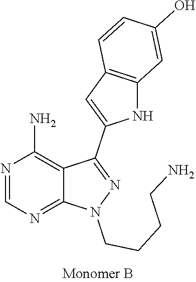 [SMILES: NCCCCn1nc(-c2cc3ccc(O)cc3[nH]2)c2c(N)ncnc21]

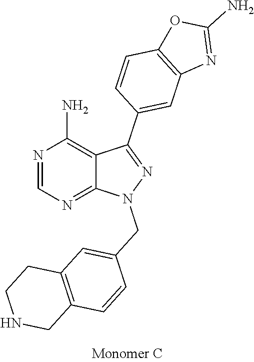 [SMILES: Nc1nc2cc(-c3nn(Cc4ccc5c(c4)CCNC5)c4ncnc(N)c34)ccc2o1]

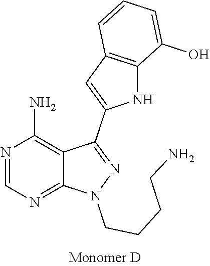 [SMILES: NCCCCn1nc(-c2cc3cccc(O)c3[nH]2)c2c(N)ncnc21]

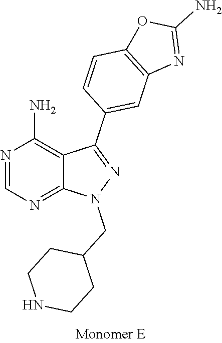 [SMILES: Nc1nc2cc(-c3nn(CC4CCNCC4)c4ncnc(N)c34)ccc2o1]

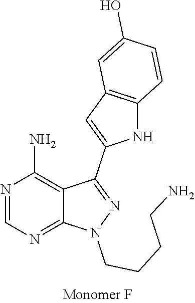 [SMILES: NCCCCn1nc(-c2cc3cc(O)ccc3[nH]2)c2c(N)ncnc21]

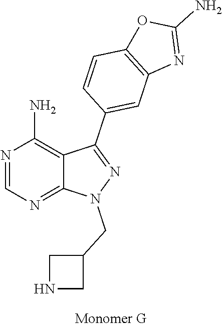 [SMILES: Nc1nc2cc(-c3nn(CC4CNC4)c4ncnc(N)c34)ccc2o1]

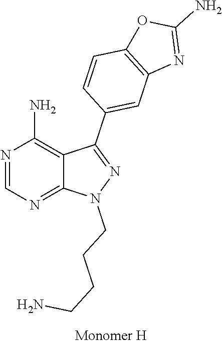 [SMILES: NCCCCn1nc(-c2ccc3oc(N)nc3c2)c2c(N)ncnc21]

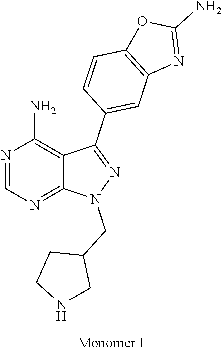 [SMILES: Nc1nc2cc(-c3nn(CC4CCNC4)c4ncnc(N)c34)ccc2o1]

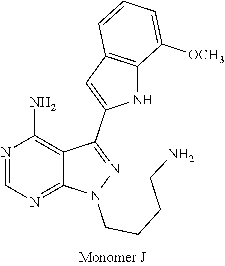 [SMILES: COc1cccc2cc(-c3nn(CCCCN)c4ncnc(N)c34)[nH]c12]

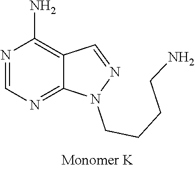 [SMILES: NCCCCn1ncc2c(N)ncnc21]

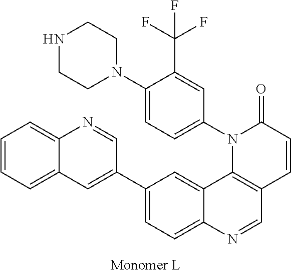 [SMILES: O=c1ccc2cnc3ccc(-c4cnc5ccccc5c4)cc3c2n1-c1ccc(N2CCNCC2)c(C(F)(F)F)c1]

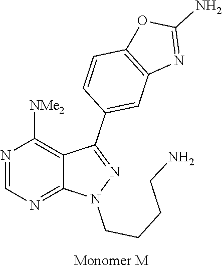 [SMILES: CN(C)c1ncnc2c1c(-c1ccc3oc(N)nc3c1)nn2CCCCN]

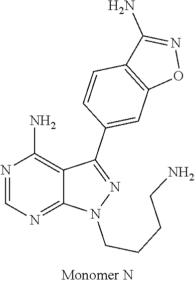 [SMILES: NCCCCn1nc(-c2ccc3c(N)noc3c2)c2c(N)ncnc21]

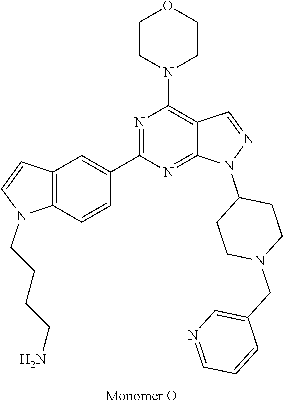

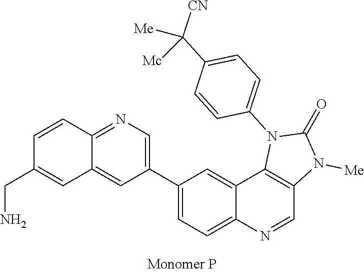

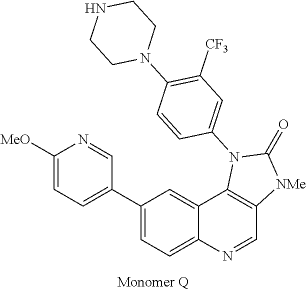 [SMILES: COc1ccc(-c2ccc3ncc4c(c3c2)n(-c2ccc(N3CCNCC3)c(C(F)(F)F)c2)c(=O)n4C)cn1]

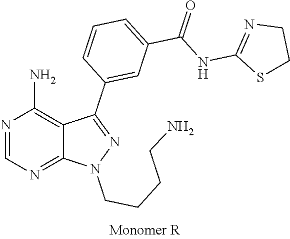 [SMILES: NCCCCn1nc(-c2cccc(C(=O)NC3=NCCS3)c2)c2c(N)ncnc21]

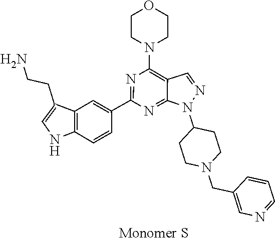

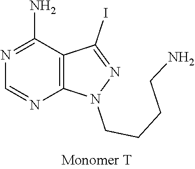 [SMILES: NCCCCn1nc(I)c2c(N)ncnc21]

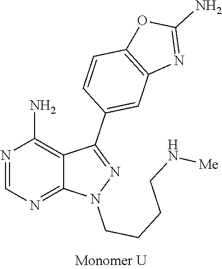 [SMILES: CNCCCCn1nc(-c2ccc3oc(N)nc3c2)c2c(N)ncnc21]

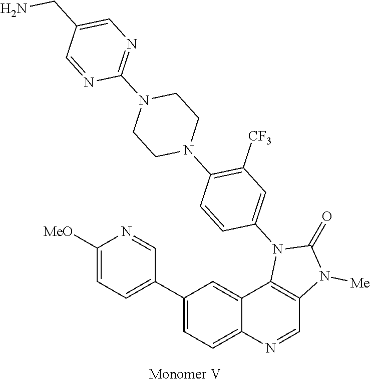 [SMILES: COc1ccc(-c2ccc3ncc4c(c3c2)n(-c2ccc(N3CCN(c5ncc(CN)cn5)CC3)c(C(F)(F)F)c2)c(=O)n4C)cn1]

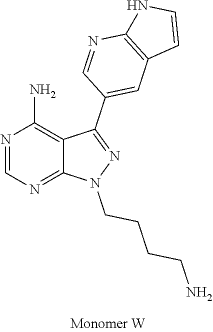 [SMILES: NCCCCn1nc(-c2cnc3[nH]ccc3c2)c2c(N)ncnc21]

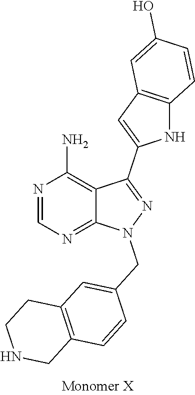 [SMILES: Nc1ncnc2c1c(-c1cc3cc(O)ccc3[nH]1)nn2Cc1ccc2c(c1)CCNC2]

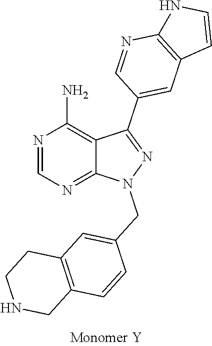 [SMILES: Nc1ncnc2c1c(-c1cnc3[nH]ccc3c1)nn2Cc1ccc2c(c1)CCNC2]

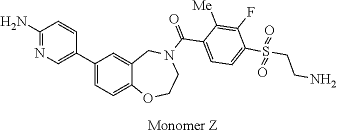 [SMILES: Cc1c(C(=O)N2CCOc3ccc(-c4ccc(N)nc4)cc3C2)ccc(S(=O)(=O)CCN)c1F]

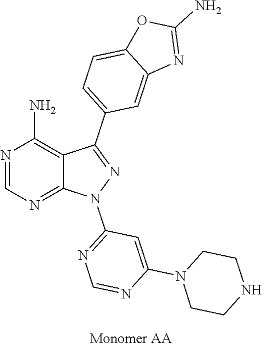 [SMILES: Nc1nc2cc(-c3nn(-c4cc(N5CCNCC5)ncn4)c4ncnc(N)c34)ccc2o1]

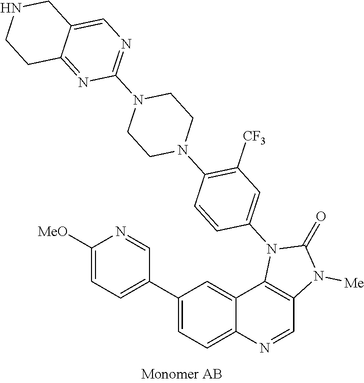 [SMILES: COc1ccc(-c2ccc3ncc4c(c3c2)n(-c2ccc(N3CCN(c5ncc6c(n5)CCNC6)CC3)c(C(F)(F)F)c2)c(=O)n4C)cn1]

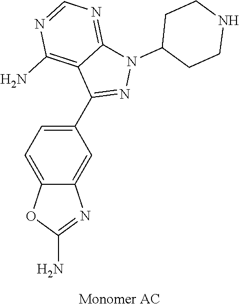 [SMILES: Nc1nc2cc(-c3nn(C4CCNCC4)c4ncnc(N)c34)ccc2o1]

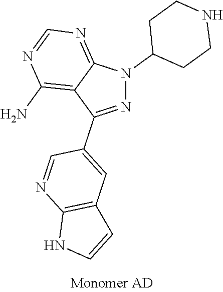 [SMILES: Nc1ncnc2c1c(-c1cnc3[nH]ccc3c1)nn2C1CCNCC1]

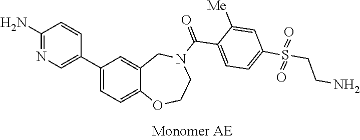 [SMILES: Cc1cc(S(=O)(=O)CCN)ccc1C(=O)N1CCOc2ccc(-c3ccc(N)nc3)cc2C1]

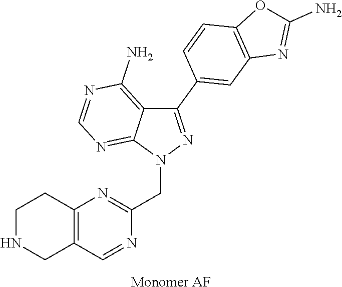

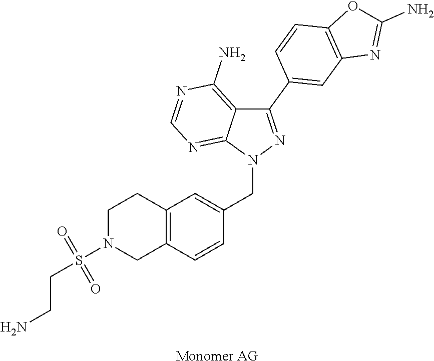 [SMILES: NCCS(=O)(=O)N1CCc2cc(Cn3nc(-c4ccc5oc(N)nc5c4)c4c(N)ncnc43)ccc2C1 ; "Monomer AG"]

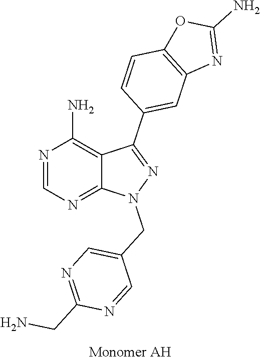 [SMILES: NCc1ncc(Cn2nc(-c3ccc4oc(N)nc4c3)c3c(N)ncnc32)cn1]

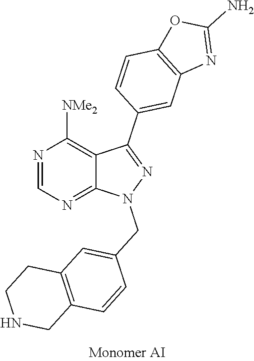 [SMILES: CN(C)c1ncnc2c1c(-c1ccc3oc(N)nc3c1)nn2Cc1ccc2c(c1)CCNC2]

TABLE 3
Active Site inhibitor monomers
Active Site Inhibitor

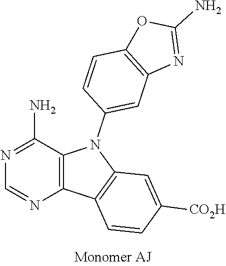 [SMILES: Nc1nc2cc(-n3c4cc(C(=O)O)ccc4c4ncnc(N)c43)ccc2o1]

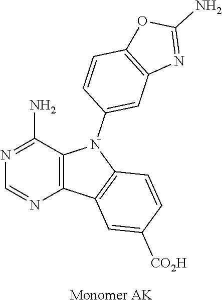 [SMILES: Nc1nc2cc(-n3c4ccc(C(=O)O)cc4c4ncnc(N)c43)ccc2o1]

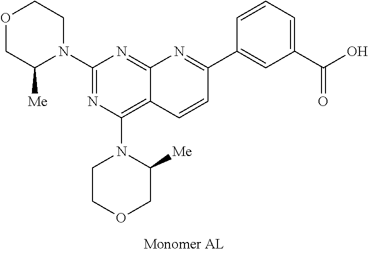 [SMILES: C[C@H]1COCCN1c1nc(N2CCOC[C@@H]2C)c2ccc(-c3cccc(C(=O)O)c3)nc2n1]

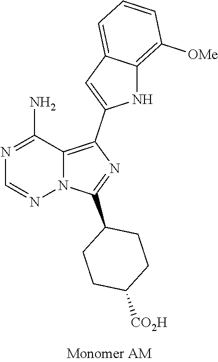 [SMILES: COc1cccc2cc(-c3nc([C@H]4CC[C@H](C(=O)O)CC4)n4ncnc(N)c34)[nH]c12]

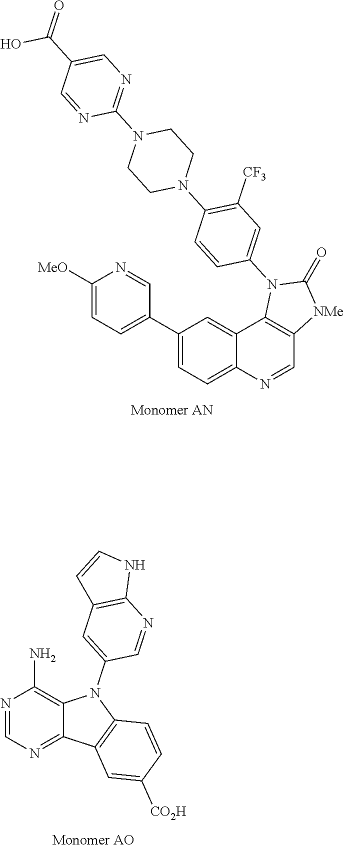 [SMILES: COc1ccc(-c2ccc3ncc4c(c3c2)n(-c2ccc(N3CCN(c5ncc(C(=O)O)cn5)CC3)c(C(F)(F)F)c2)c(=O)n4C)cn1 ; Nc1ncnc2c3cc(C(=O)O)ccc3n(-c3cnc4[nH]ccc4c3)c12 ; "Monomer AO"]

TABLE 4
Amine containing pre- and post-linkers.
Amide containing block

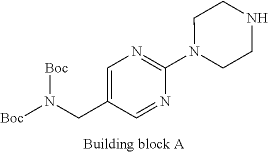 [SMILES: CC(C)(C)OC(=O)N(Cc1cnc(N2CCNCC2)nc1)C(=O)OC(C)(C)C]

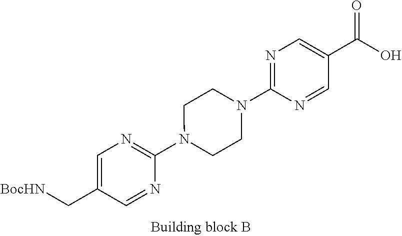 [SMILES: CC(C)(C)OC(=O)NCc1cnc(N2CCN(c3ncc(C(=O)O)cn3)CC2)nc1]

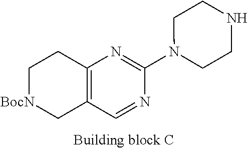 [SMILES: CC(C)(C)OC(=O)N1CCc2nc(N3CCNCC3)ncc2C1]

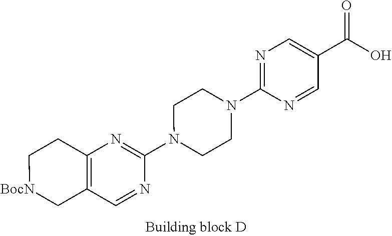

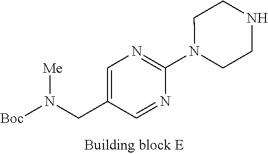 [SMILES: CN(Cc1cnc(N2CCNCC2)nc1)C(=O)OC(C)(C)C]

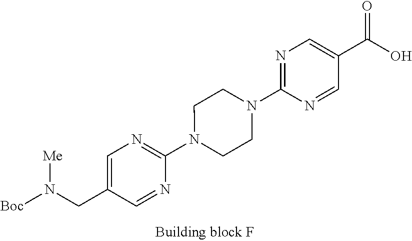 [SMILES: CN(Cc1cnc(N2CCN(c3ncc(C(=O)O)cn3)CC2)nc1)C(=O)OC(C)(C)C]

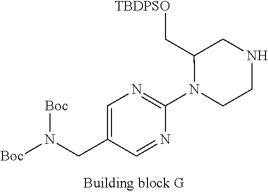

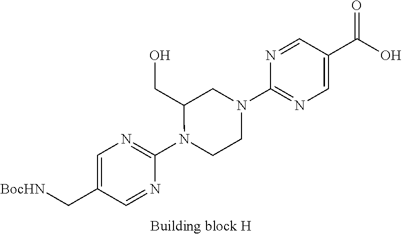 [SMILES: CC(C)(C)OC(=O)NCc1cnc(N2CCN(c3ncc(C(=O)O)cn3)CC2CO)nc1]

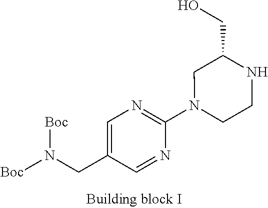 [SMILES: CC(C)(C)OC(=O)N(Cc1cnc(N2CCN[C@@H](CO)C2)nc1)C(=O)OC(C)(C)C]

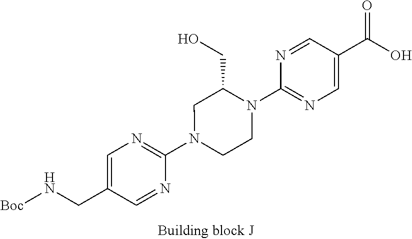 [SMILES: CC(C)(C)OC(=O)NCc1cnc(N2CCN(c3ncc(C(=O)O)cn3)[C@@H](CO)C2)nc1]

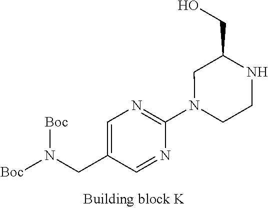 [SMILES: CC(C)(C)OC(=O)N(Cc1cnc(N2CCN[C@H](CO)C2)nc1)C(=O)OC(C)(C)C]

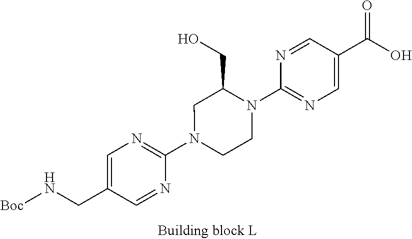 [SMILES: CC(C)(C)OC(=O)NCc1cnc(N2CCN(c3ncc(C(=O)O)cn3)[C@H](CO)C2)nc1]

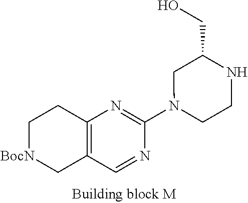 [SMILES: CC(C)(C)OC(=O)N1CCc2nc(N3CCN[C@@H](CO)C3)ncc2C1]

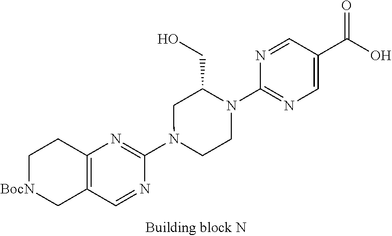 [SMILES: CC(C)(C)OC(=O)N1CCc2nc(N3CCN(c4ncc(C(=O)O)cn4)[C@@H](CO)C3)ncc2C1]

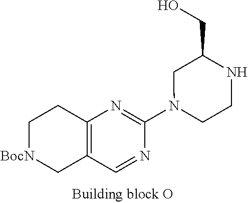 [SMILES: CC(C)(C)OC(=O)N1CCc2nc(N3CCN[C@H](CO)C3)ncc2C1]

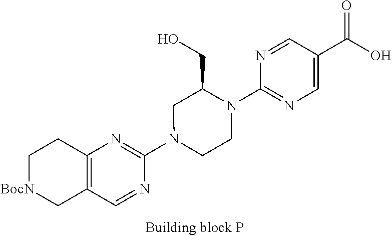 [SMILES: CC(C)(C)OC(=O)N1CCc2nc(N3CCN(c4ncc(C(=O)O)cn4)[C@H](CO)C3)ncc2C1]

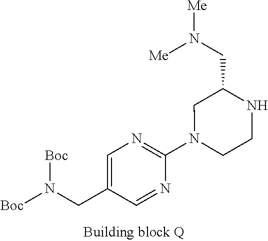 [SMILES: CN(C)C[C@H]1CN(c2ncc(CN(C(=O)OC(C)(C)C)C(=O)OC(C)(C)C)cn2)CCN1]

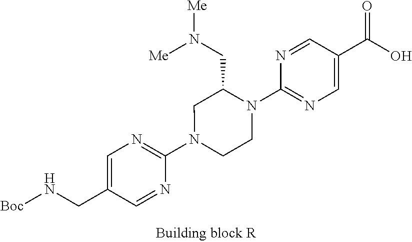 [SMILES: CN(C)C[C@H]1CN(c2ncc(CNC(=O)OC(C)(C)C)cn2)CCN1c1ncc(C(=O)O)cn1]

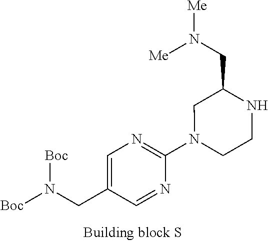 [SMILES: CN(C)C[C@@H]1CN(c2ncc(CN(C(=O)OC(C)(C)C)C(=O)OC(C)(C)C)cn2)CCN1]

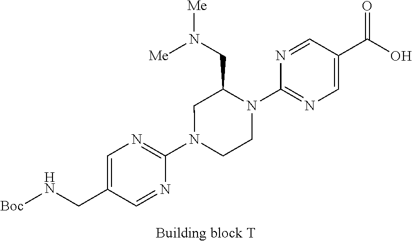 [SMILES: CN(C)C[C@@H]1CN(c2ncc(CNC(=O)OC(C)(C)C)cn2)CCN1c1ncc(C(=O)O)cn1]

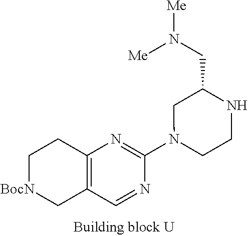 [SMILES: CN(C)C[C@H]1CN(c2ncc3c(n2)CCN(C(=O)OC(C)(C)C)C3)CCN1]

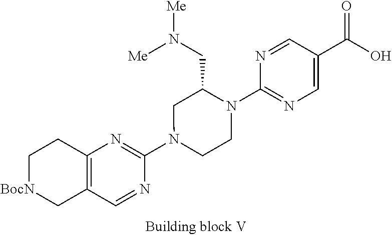 [SMILES: CN(C)C[C@H]1CN(c2ncc3c(n2)CCN(C(=O)OC(C)(C)C)C3)CCN1c1ncc(C(=O)O)cn1]

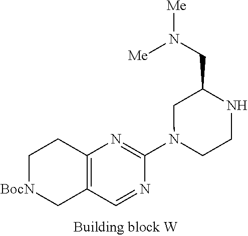 [SMILES: CN(C)C[C@@H]1CN(c2ncc3c(n2)CCN(C(=O)OC(C)(C)C)C3)CCN1]

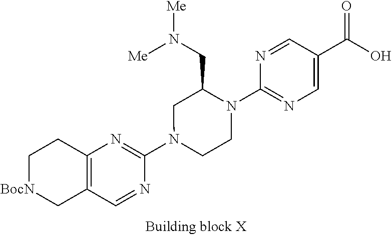 [SMILES: CN(C)C[C@@H]1CN(c2ncc3c(n2)CCN(C(=O)OC(C)(C)C)C3)CCN1c1ncc(C(=O)O)cn1]

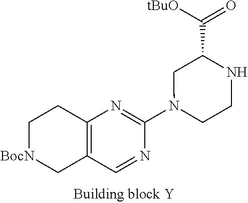 [SMILES: CC(C)(C)OC(=O)[C@H]1CN(c2ncc3c(n2)CCN(C(=O)OC(C)(C)C)C3)CCN1]

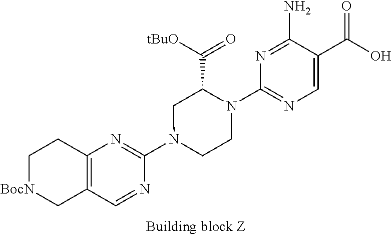 [SMILES: CC(C)(C)OC(=O)[C@H]1CN(c2ncc3c(n2)CCN(C(=O)OC(C)(C)C)C3)CCN1c1ncc(C(=O)O)c(N)n1]

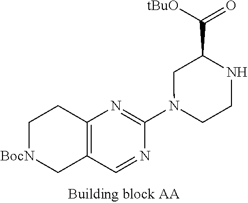 [SMILES: CC(C)(C)OC(=O)[C@@H]1CN(c2ncc3c(n2)CCN(C(=O)OC(C)(C)C)C3)CCN1]

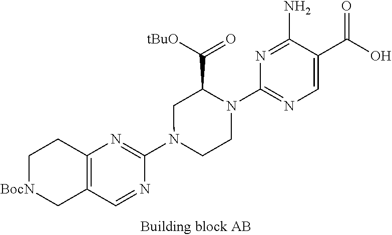 [SMILES: CC(C)(C)OC(=O)[C@@H]1CN(c2ncc3c(n2)CCN(C(=O)OC(C)(C)C)C3)CCN1c1ncc(C(=O)O)c(N)n1]

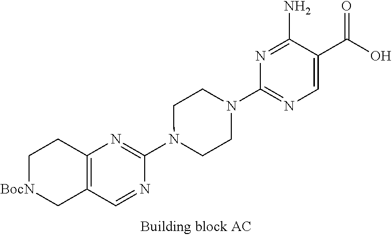 [SMILES: CC(C)(C)OC(=O)N1CCc2nc(N3CCN(c4ncc(C(=O)O)c(N)n4)CC3)ncc2C1]

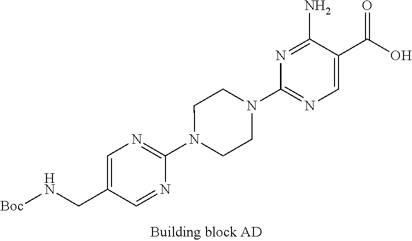 [SMILES: CC(C)(C)OC(=O)NCc1cnc(N2CCN(c3ncc(C(=O)O)c(N)n3)CC2)nc1]

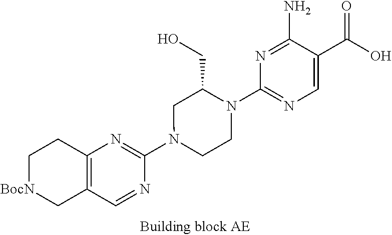 [SMILES: CC(C)(C)OC(=O)N1CCc2nc(N3CCN(c4ncc(C(=O)O)c(N)n4)[C@@H](CO)C3)ncc2C1]

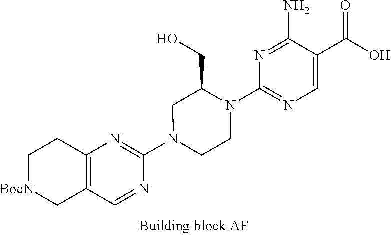 [SMILES: CC(C)(C)OC(=O)N1CCc2nc(N3CCN(c4ncc(C(=O)O)c(N)n4)[C@H](CO)C3)ncc2C1]

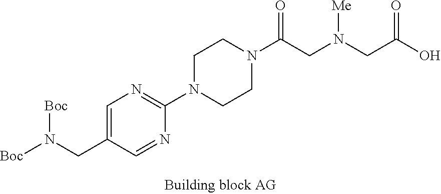 [SMILES: CN(CC(=O)O)CC(=O)N1CCN(c2ncc(CN(C(=O)OC(C)(C)C)C(=O)OC(C)(C)C)cn2)CC1]

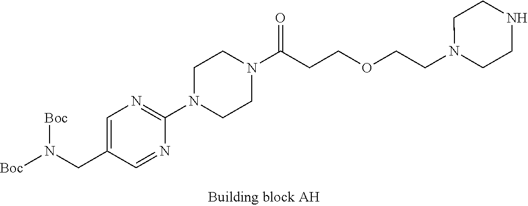 [SMILES: CC(C)(C)OC(=O)N(Cc1cnc(N2CCN(C(=O)CCOCCN3CCNCC3)CC2)nc1)C(=O)OC(C)(C)C]

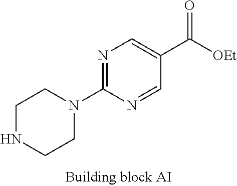 [SMILES: CCOC(=O)c1cnc(N2CCNCC2)nc1]

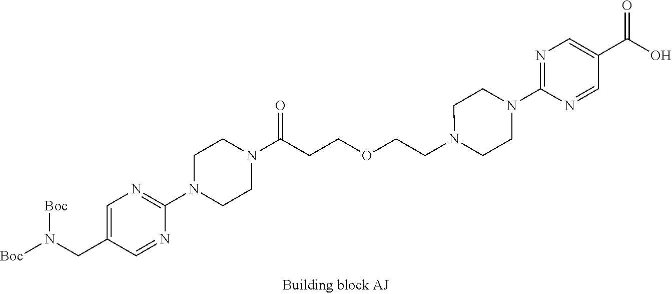 [SMILES: CC(C)(C)OC(=O)N(Cc1cnc(N2CCN(C(=O)CCOCCN3CCN(c4ncc(C(=O)O)cn4)CC3)CC2)nc1)C(=O)OC(C)(C)C]

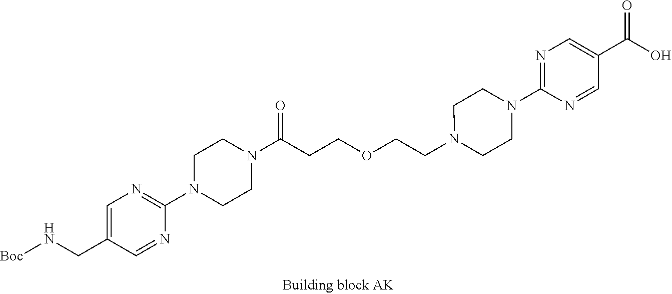 [SMILES: CC(C)(C)OC(=O)NCc1cnc(N2CCN(C(=O)CCOCCN3CCN(c4ncc(C(=O)O)cn4)CC3)CC2)nc1]

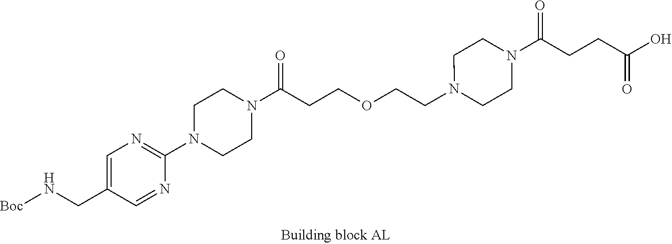 [SMILES: CC(C)(C)OC(=O)NCc1cnc(N2CCN(C(=O)CCOCCN3CCN(C(=O)CCC(=O)O)CC3)CC2)nc1]

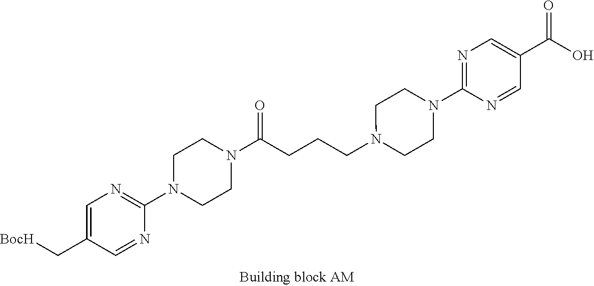

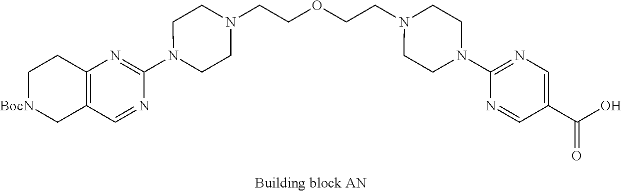 [SMILES: CC(C)(C)OC(=O)N1CCc2nc(N3CCN(CCOCCN4CCN(c5ncc(C(=O)O)cn5)CC4)CC3)ncc2C1]

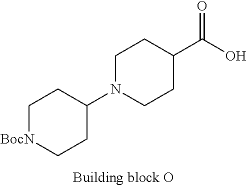 [SMILES: CC(C)(C)OC(=O)N1CCC(N2CCC(C(=O)O)CC2)CC1]

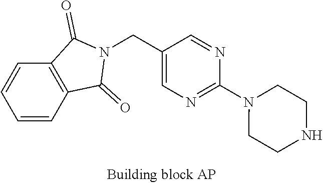 [SMILES: O=C1c2ccccc2C(=O)N1Cc1cnc(N2CCNCC2)nc1]

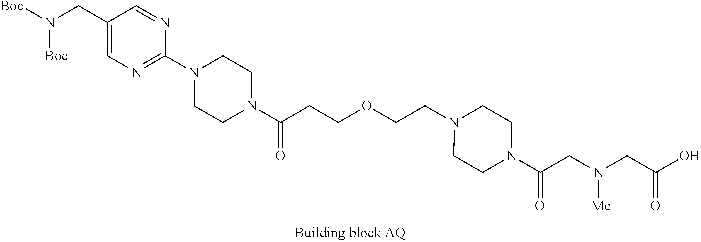 [SMILES: CN(CC(=O)O)CC(=O)N1CCN(CCOCCC(=O)N2CCN(c3ncc(CN(C(=O)OC(C)(C)C)C(=O)OC(C)(C)C)cn3)CC2)CC1]

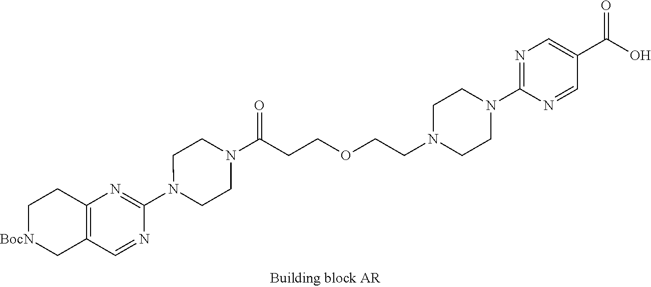 [SMILES: CC(C)(C)OC(=O)N1CCc2nc(N3CCN(C(=O)CCOCCN4CCN(c5ncc(C(=O)O)cn5)CC4)CC3)ncc2C1]

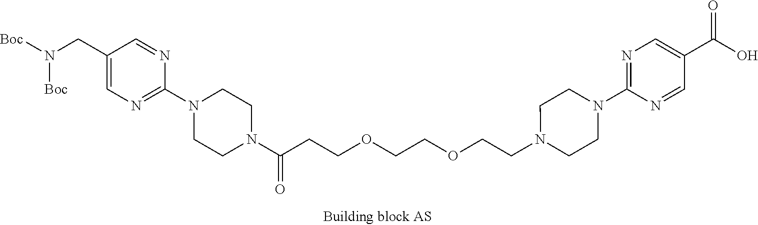 [SMILES: CC(C)(C)OC(=O)N(Cc1cnc(N2CCN(C(=O)CCOCCOCCN3CCN(c4ncc(C(=O)O)cn4)CC3)CC2)nc1)C(=O)OC(C)(C)C]

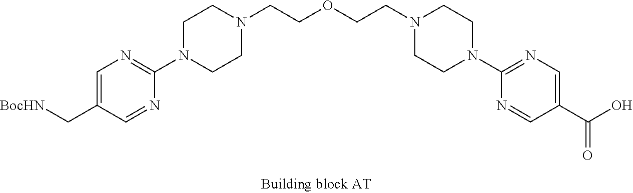 [SMILES: CC(C)(C)OC(=O)NCc1cnc(N2CCN(CCOCCN3CCN(c4ncc(C(=O)O)cn4)CC3)CC2)nc1]

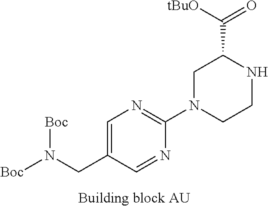 [SMILES: CC(C)(C)OC(=O)[C@H]1CN(c2ncc(CN(C(=O)OC(C)(C)C)C(=O)OC(C)(C)C)cn2)CCN1]

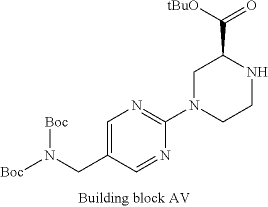 [SMILES: CC(C)(C)OC(=O)[C@@H]1CN(c2ncc(CN(C(=O)OC(C)(C)C)C(=O)OC(C)(C)C)cn2)CCN1]

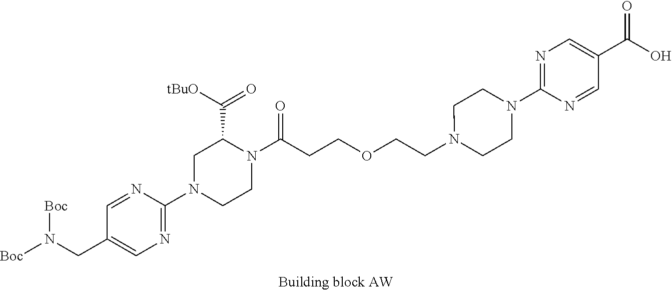 [SMILES: CC(C)(C)OC(=O)[C@H]1CN(c2ncc(CN(C(=O)OC(C)(C)C)C(=O)OC(C)(C)C)cn2)CCN1C(=O)CCOCCN1CCN(c2ncc(C(=O)O)cn2)CC1]

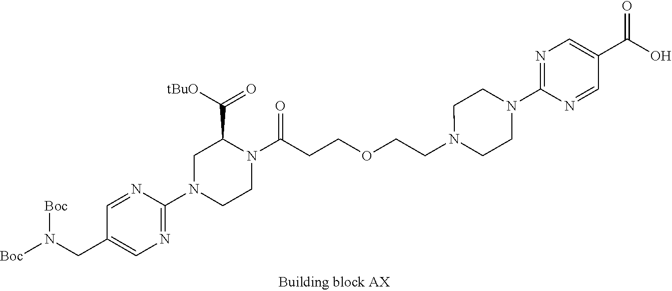 [SMILES: CC(C)(C)OC(=O)[C@@H]1CN(c2ncc(CN(C(=O)OC(C)(C)C)C(=O)OC(C)(C)C)cn2)CCN1C(=O)CCOCCN1CCN(c2ncc(C(=O)O)cn2)CC1]

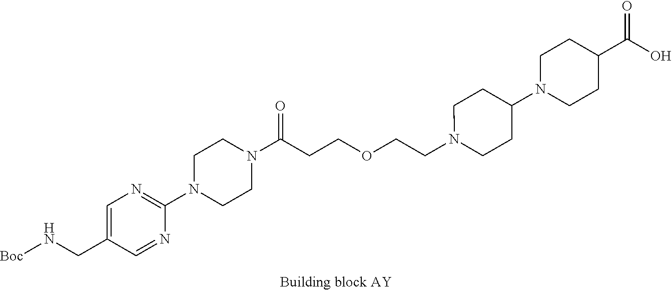 [SMILES: CC(C)(C)OC(=O)NCc1cnc(N2CCN(C(=O)CCOCCN3CCC(N4CCC(C(=O)O)CC4)CC3)CC2)nc1]

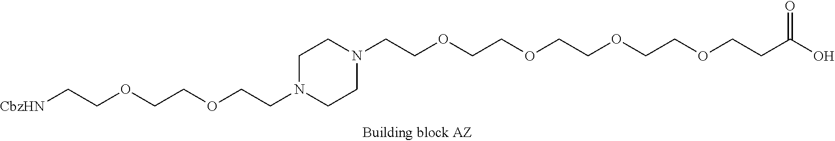 [SMILES: O=C(O)CCOCCOCCOCCOCCN1CCN(CCOCCOCCNC(=O)OCc2ccccc2)CC1]

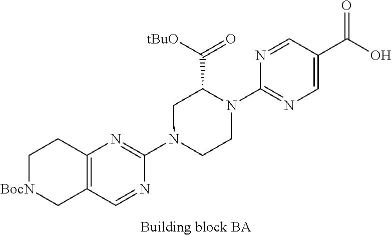 [SMILES: CC(C)(C)OC(=O)[C@H]1CN(c2ncc3c(n2)CCN(C(=O)OC(C)(C)C)C3)CCN1c1ncc(C(=O)O)cn1]

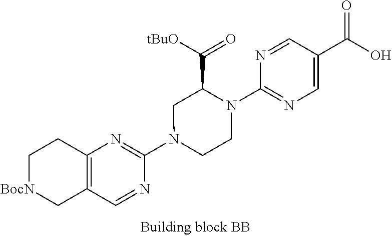 [SMILES: CC(C)(C)OC(=O)[C@@H]1CN(c2ncc3c(n2)CCN(C(=O)OC(C)(C)C)C3)CCN1c1ncc(C(=O)O)cn1]

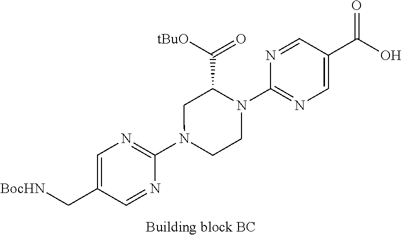 [SMILES: CC(C)(C)OC(=O)NCc1cnc(N2CCN(c3ncc(C(=O)O)cn3)[C@@H](C(=O)OC(C)(C)C)C2)nc1]

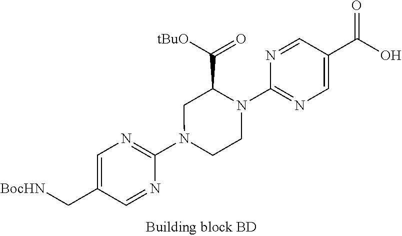 [SMILES: CC(C)(C)OC(=O)NCc1cnc(N2CCN(c3ncc(C(=O)O)cn3)[C@H](C(=O)OC(C)(C)C)C2)nc1]

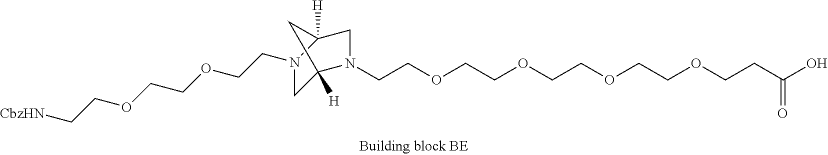 [SMILES: O=C(O)CCOCCOCCOCCOCCN1C[C@@H]2C[C@H]1CN2CCOCCOCCNC(=O)OCc1ccccc1]

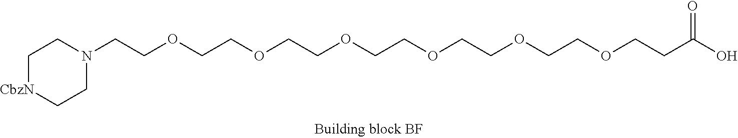 [SMILES: O=C(O)CCOCCOCCOCCOCCOCCOCCN1CCN(C(=O)OCc2ccccc2)CC1]

### Preparation of Active Site Inhibitor Monomers

### Monomer A. 5-(4-amino-1-(4-(aminomethyl)benzyl)-1H-pyrazolo[3,4-d]pyrimidin-3-yl)benzo[d]oxazol-2-amine trifluoroacetic acid salt

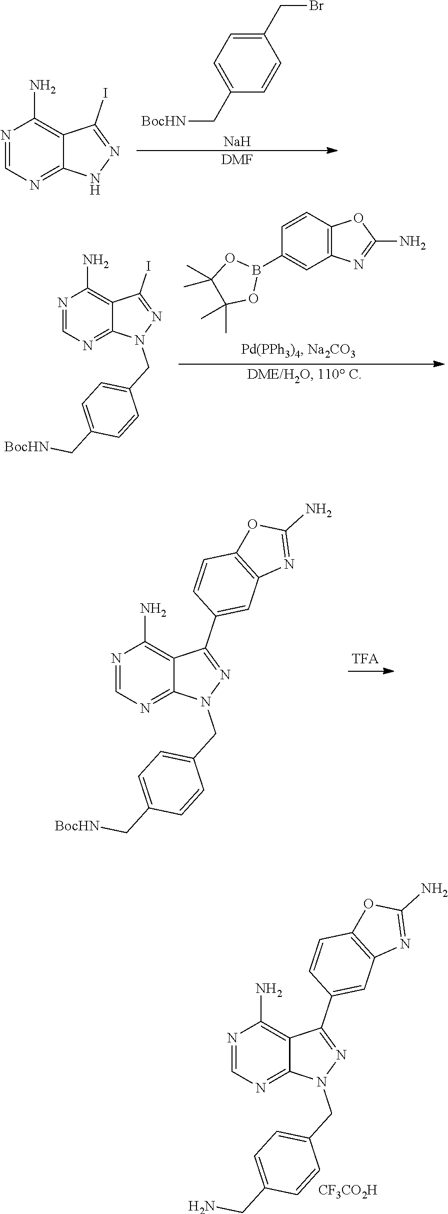

### Step 1: Synthesis of tert-butyl 4-((4-amino-3-iodo-1H-pyrazolo[3,4-d]pyrimidin-1-yl)methyl)benzylcarbamate

To a solution of 3-iodo-1H-pyrazolo[3,4-d]pyrimidin-4-amine (3.8 g, 14.56 mmol. 1.0 equiv) in DMF (20 mL) was added NaH (582.27 mg, 14.56 mmol, 60 wt. %, 1.0 equiv) at 0° C. and the reaction solution was stirred at this temperature for 30 min, then tert-butyl 4-(bromomethyl)benzylcarbamate (4.59 g, 15.29 mmol, 1.05 equiv) was added to the reaction at 0° C. and the reaction solution was stirred at room temperature for 2 h. The solution was poured into H₂O (80 mL) and the solid that precipitated out was filtered. The solid cake was washed with H₂O (2×10 mL) and then dried under reduced pressure to give tert-butyl 4-((4-amino-3-iodo-1H-pyrazolo[3,4-d]pyrimidin-1-yl)methyl)benzylcarbamate (5 g, 53% yield) as a yellow solid. LCMS (ESI) m/z: [M+Na] calcd for C₁₈H₂₁IN₆O₂: 503.07; found 503.2.

### Step 2: Synthesis of tert-butyl 4-((4-amino-3-(2-aminobenzo[d]oxazol-5-yl)-1H-pyrazolo[3,4-d]pyrimidin-1-yl)methyl)benzylcarbamate

To a bi-phasic suspension of tert-butyl 4-(4-amino-3-iodo-1H-pyrazolo[3,4-d]pyrimidin-1-yl)methyl)benzylcarbamate (5 g, 7.68 mmol, 1.0 equiv), 5-(4,4,5,5-tetramethyl-1,3,2-dioxaborolan-2-yl)benzo[d]oxazol-2-amine (2.40 g, 9.22 mmol, 1.2 equiv) and Pd(PPh₃)₄ (887.66 mg, 768.16 μmol, 0.1 equiv) in DME (100 mL) and H₂O (50 mL) was added Na₂CO₃ (1.91 g, 23.04 mmol, 3.0 equiv) at room temperature under N₂. The mixture was stirred at 110° C. for 3 h. The reaction mixture was cooled to room temperature and filtered, the filtrate was extracted by EtOAc (3×50 mL). The organic phases were combined and washed with brine (10 mL), dried over Na₂SO₄, filtered, and the filtrate was concentrated under reduced pressure to give a residue. The residue was purified by silica gel chromatography (0→20% MeOH/EtOAc) to give tert-butyl 4-((4-amino-3-(2-aminobenzo[d]oxazol-5-yl)-1H-pyrazolo[3,4-d]pyrimidin-1-yl)methyl)benzylcarbamate (4.5 g, 82% yield) as a yellow solid. LCMS (ESI) m/z: [M+H] calcd for C₂₅H₂₆NO₃: 487.22; found 487.2.

### Step 3: Synthesis of 5-(4-amino-1-(4-(aminomethyl)benzyl)-1H-pyrazolo[3,4-d]pyrimidin-3-yl)benzo[d]oxazol-2-amine

To a solution of tert-butyl 4-((4-amino-3-(2-aminobenzo[d]oxazol-5-yl)-1H-pyrazolo[3,4-d]pyrimidin-1-yl)methyl)benzylcarbamate (4.5 g, 6.29 mmol, 1.0 equiv) in DCM (50 mL) was added TFA (30.80 g, 270.12 mmol, 20 mL, 42.95 equiv) at 0° C. The reaction solution was stirred at room temperature for 2 h. The reaction solution was concentrated under reduced pressure to give a residue, which was dissolved in 10 mL of MeCN, then poured into MTBE (100 mL). The solid that precipitated was then filtered and the solid cake was dried under reduced pressure to give 5-[4-amino-1-[[4-(aminomethyl)phenyl]methyl]pyrazolo[3,4-d]pyrimidin-3-yl]-1,3-benzoxazol-2-amine (2.22 g, 71% yield, TFA) as a yellow solid. LCMS (ESI) m/z: [M+H] calcd for C₂₀H₁₈N₈O: 387.16; found 387.1.

### Monomer B. 2-(4-amino-1-(4-aminobutyl)-1H-pyrazolo[3,4-d]pyrimidin-3-yl)-1H-indol-6-ol trifluoroacetic acid salt

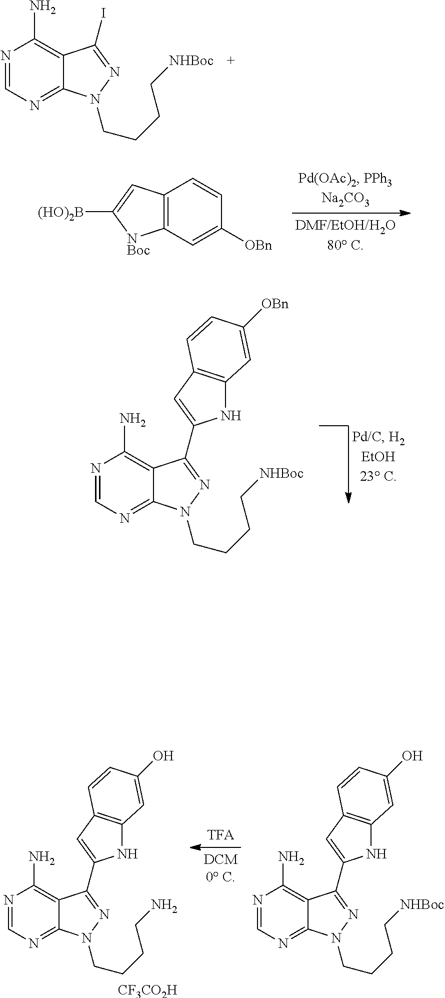

### Step 1: Synthesis of tert-butyl N-(4-{4-amino-3-[6-(benzyloxy)-1H-indol-2-yl]-1H-pyrazolo[3,4-d]pyrimidin-1-yl}butyl)carbamate

To a mixture of tert-butyl (4-(4-amino-3-iodo-1H-pyrazolo[3,4-d]pyrimidin-1-yl)butyl)carbamate (300 mg, 694 μmol, 1.0 equiv) and (6-(benzyloxy)-1-(tert-butoxycarbonyl)-1H-indol-2-yl)boronic acid (763 mg, 2.08 mmol, 3.0 equiv) in DMF (2.6 mL), EtOH (525 L), and H₂O (350 μL) were added Pd(OAc)₂ (15.5 mg, 69 μmol, 0.1 equiv), triphenylphosphine (36.1 mg, 138 μmol, 0.2 equiv), and sodium carbonate (440 mg, 4.16 mmol, 6.0 equiv). The reaction was heated at 80° C. for 20 h, cooled to room temperature, and quenched with H₂O (10 mL) and EtOAc (10 mL). The mixture was transferred to a separatory funnel and the aqueous phase was extracted with EtOAc (3×20 mL). The combined organic phase was washed with sat. aq. NaCl (1×20 mL), dried over Na₂SO₄, filtered, and concentrated under reduced pressure. The crude material was purified by silica gel chromatography (20→85% EtOAc/heptane) to provide the product (201 mg, 46% yield) as an orange solid. LCMS (ESI) m/z: [M+H] calcd for C₂₉H₃₃N₇O₃: 528.27; found 528.2.

### Step 2: Synthesis of tert-butyl (4-(4-amino-3-(6-hydroxy-1H-indol-2-yl)-1H-pyrazolo[3,4-d]pyrimidin-1-yl)butyl)carbamate

To a solution of tert-butyl N-(4-{4-amino-3-[6-(benzyloxy)-1H-indol-2-yl]-1H-pyrazolo[3,4-d]pyrimidin-1-yl}butyl)carbamate (1.0 equiv) in EtOH is added Pd/C (10 mol %). The reaction is purged with H₂ and the reaction allowed to stir under an atmosphere of H₂ until consumption of starting material, as determined by LCMS. The reaction is then diluted with EtOAc, filtered over Celite, and concentrated under reduced pressure. The resultant residue is purified by silica gel chromatography to afford the desired product.

### Step 3: Synthesis of 2-(4-amino-1-(4-aminobutyl)-H-pyrazolo[3,4-d]pyrimidin-3-yl)-1H-indol-6-ol

To a solution of tert-butyl (4-(4-amino-3-(6-hydroxy-1H-indol-2-yl)-1H-pyrazolo[3,4-d]pyrimidin-1-yl)butyl)carbamate (1.0 equiv) in anhydrous DCM is added TFA (50 equiv.) dropwise at 0° C. The reaction is stirred at 0° C. and warmed to room temperature. Once the reaction is complete, as determined by LCMS, the reaction is concentrated under reduced pressure. The residue is triturated with MeCN, then dripped into MTBE over 10 min. The supernatant is removed and the precipitate is collected by filtration under N₂ to give 2-(4-amino-1-(4-aminobutyl)-1H-pyrazolo[3,4-d]pyrimidin-3-yl)-1H-indol-6-ol.

### Monomer C. 5-(4-amino-1-((1,2,3,4-tetrahydroisoquinolin-6-yl)methyl)-1H-pyrazolo[3,4-d]pyrimidin-3-yl)benzo[d]oxazol-2-amine trifluoroacetic acid salt

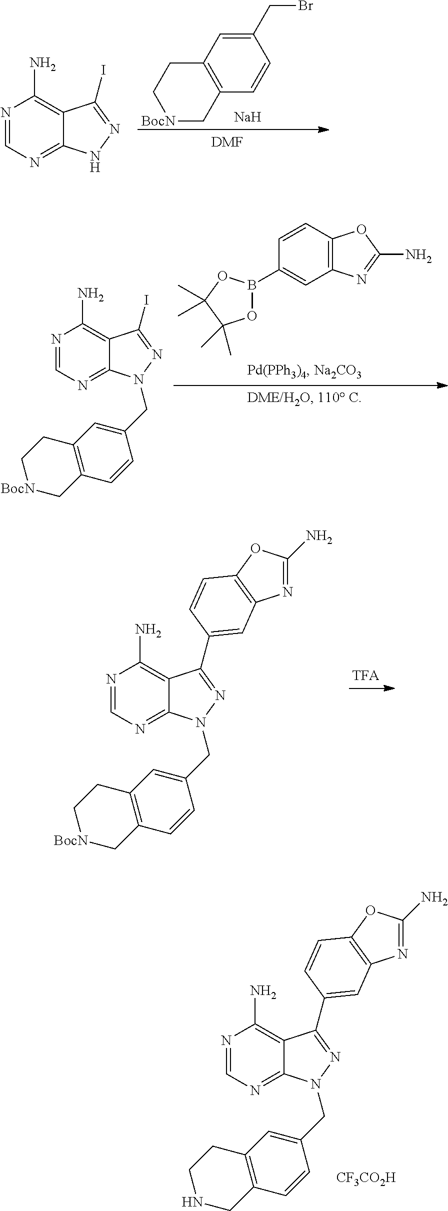

### Step 1: Synthesis of tert-butyl 6-((4-amino-3-iodo-1H-pyrazolo[3,4-d]pyrimidin-1-yl)methyl)-3,4-dihydroisoquinoline-2(1H)-carboxylate

To a suspension of 3-iodo-1H-pyrazolo[3,4-d]pyrimidin-4-amine (5 g, 19.16 mmol, 1.0 equiv) in DMF (50.0 mL) was added NaH (766.22 mg, 19.16 mmol, 60 wt. %, 1.0 equiv) at 4° C. The mixture was stirred at 4° C. for 30 min. To the reaction mixture was added tert-butyl 6-(bromomethyl)-3,4-dihydroisoquinoline-2(1H)-carboxylate (6.87 g, 21.07 mmol, 1.1 equiv) in DMF (30 mL) at 4° C. The mixture was stirred at room temperature for 2 h. The mixture was then cooled to 4° C. and H₂O (400 mL) was added and the mixture was stirred for 30 min. The resulting precipitate was collected by filtration to give crude tert-butyl 6-((4-amino-3-iodo-1H-pyrazolo[3,4-d]pyrimidin-1-yl)methyl)-3,4-dihydroisoquinoline-2(1H)-carboxylate (9.7 g, 76% yield) as a light yellow solid. The crude product was used for the next step directly.

### Step 2: Synthesis of tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate

To a bi-phasic suspension of tert-butyl 6-((4-amino-3-iodo-1H-pyrazolo[3,4-d]pyrimidin-1-yl)methyl)-3,4-dihydroisoquinoline-2(1H)-carboxylate (9.7 g, 14.63 mmol, 1.0 equiv), 5-(4,4,5,5-tetramethyl-1,3,2-dioxaborolan-2-yl)benzo[d]oxazol-2-amine (4.57 g, 17.55 mmol, 1.2 equiv), and Na₂CO₃ (7.75 g, 73.14 mmol, 5.0 equiv) in DME (120.0 mL) and H₂O (60 mL) was added Pd(PPh₃)₄ (1.69 g, 1.46 mmol, 0.1 equiv) at room temperature under N₂. The mixture was stirred at 110° C. for 3 h. The reaction mixture was then cooled to room temperature and partitioned between EtOAc (100 mL) and H₂O (100 mL). The aqueous layer was separated and extracted with EtOAc (2×60 mL). The organic layers were combined, washed with brine (80 mL) and dried over anhydrous Na₂SO₄, filtered and the filtrate was concentrated under reduced pressure. The residue was purified by silica gel chromatography (1→100% EtOAc/petroleum ether, then 20-50% MeOH/EtOAc) to afford tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (4.5 g, 58% yield) as a light yellow solid.

### Step 3: Synthesis of 5-(4-amino-1-((1,2,3,4-tetrahydroisoquinolin-6-yl)methyl)-1H-pyrazolo[3,4-d]pyramidin-3-yl)benzo[d]oxazol-2-amine

To neat TFA (32.5 mL, 438.97 mmol, 50.0 equiv) was added tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (4.5 g, 8.78 mmol, 1.0 equiv) at room temperature. The mixture was stirred for 30 min and then concentrated under reduced pressure. The oily residue was triturated with MeCN (8 mL), then dripped into MTBE (350 mL) over 10 min. The supernatant was removed and then the precipitate was collected by filtration under N₂ to give 5-(4-amino-1-((1,2,3,4-tetrahydroisoquinolin-6-yl)methyl)-1H-pyrazolo[3,4-d]pyrimidin-3-yl)benzo[d]oxazol-2-amine (5.72 g, over 100% yield, TFA) as a light pink solid. LCMS (ESI) m/z: [M+H] calcd for C₂₂H₂₀N₈O: 413.18; found 413.2.

### Monomer D. 2-(4-amino-1-(4-aminobutyl)-1H-pyrazolo[3,4-d]pyrimidin-3-yl)-1H-indol-7-ol trifluoroacetic acid salt

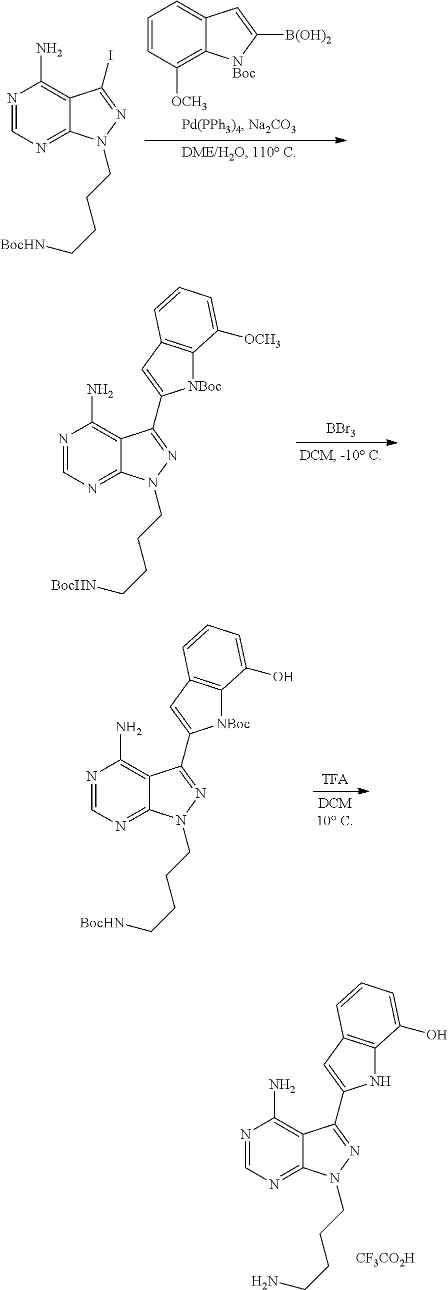

### Step 1: Synthesis of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate

To a mixture of tert-butyl (4-(4-amino-3-iodo-1H-pyrazolo[3,4-d]pyrimidin-1-yl)butyl)carbamate (1.0 equiv) and (1-(tert-butoxycarbonyl)-7-methoxy-1H-indol-2-yl)boronic acid (3.0 equiv) in DME and H₂O is added Pd(PPh₃)₄ (0.1 equiv) and sodium carbonate (6.0 equiv). The reaction is heated at 80° C. until completion, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 2: Synthesis of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate

To a solution of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate (1.0 equiv) in DCM at −10° C. is added BBr₃ (2.0 equiv). The reaction is allowed to stir until consumption of starting material, as determined by LCMS. The reaction is quenched by slow addition of sat. aq. NaHCO₃, transferred to a separatory funnel and the mixture is extracted with DCM. The organic phase is washed with sat. aq. NaCl, dried over Na₂SO₄, filtered, and concentrated under reduced pressure. The desired product is isolated after chromatography on silica gel.

### Step 3: Synthesis of 2-(4-amino-1-(4-aminobutyl)-1H-pyrazolo[3,4-d]pyrimidin-3-yl)-1H-indol-7-ol

To a solution of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate (1.0 equiv) in DCM at 0° C. is added TFA dropwise. The reaction is stirred at 0° C. and warmed to room temperature. Once the reaction is complete, as determined by LCMS, the reaction is concentrated under reduced pressure. The residue is triturated with MeCN, then dripped into MTBE over 10 min. The supernatant is removed and the precipitate is collected by filtration under N₂ to give 2-(4-amino-1-(4-aminobutyl)-1H-pyrazolo[3,4-d]pyrimidin-3-yl)-1H-indol-7-ol.

### Monomer E. 5-(4-amino-1-(piperidin-4-ylmethyl)-1H-pyrazolo[3,4-d]pyrimidin-3-yl)benzo[d]oxazol-2-amine trifluoroacetic acid salt

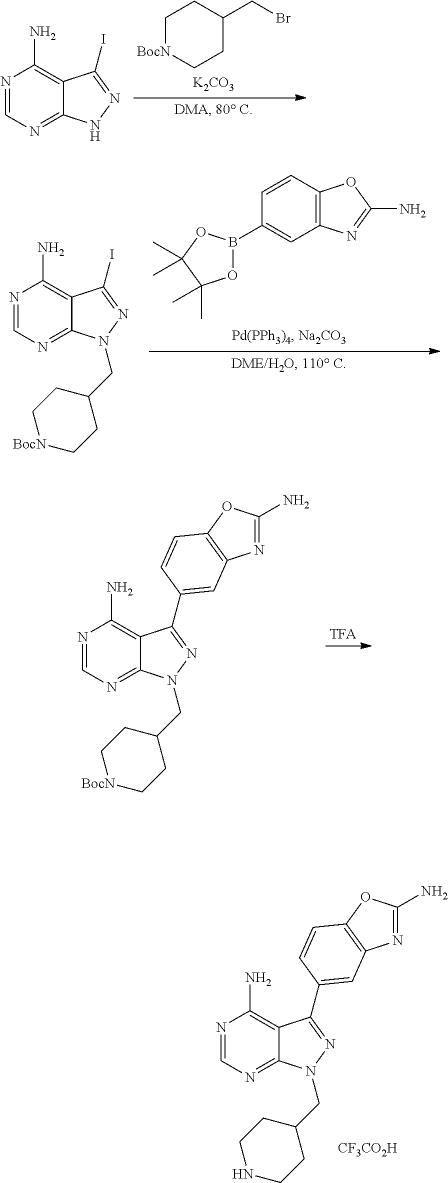

Step 1: Synthesis of tert-butyl 4-((4-amino-3-iodo-1H-pyrazolo[3,4-d]pyrimidin-1-yl)methyl)piperidine-1-carboxylate

To a solution of 3-iodo-1H-pyrazolo[3,4-d]pyrimidin-4-amine (3 g, 11.49 mmol, 1.0 equiv) in DMA (30 mL) was added tert-butyl 4-(bromomethyl)piperidine-1-carboxylate (3.36 g, 12.07 mmol, 1.05 equiv) and K₂CO₃ (4.77 g, 34.48 mmol, 3.0 equiv), then the reaction was stirred at 80° C. for 3 h. The reaction mixture was filtered to remove K₂CO₃ and the filtrate was poured into H₂O (200 mL). A solid precipitated was then filtered to give tert-butyl 4-((4-amino-3-iodo-1H-pyrazolo[3,4-d]pyrimidin-1-yl)methyl)piperidine-1-carboxylate (3 g, 57% yield) as a light yellow solid. LCMS (ESI) m/z: [M+H] calcd for C₁₆H₂₃IN₆O₂: 459.10; found 459.1.

### Step 2: Synthesis of tert-butyl 4-((4-amino-3-(2-aminobenzo[d]oxazol-5-yl)-1H-pyrazolo[3,4-d]pyrimidin-1-yl)methyl)piperidine-1-carboxylate

To a bi-phasic suspension of tert-butyl 4-((4-amino-3-iodo-1H-pyrazolo[3,4-d]pyrimidin-1-yl)methyl)piperidine-1-carboxylate (3 g, 6.55 mmol, 1.0 equiv) and 5-(4,4,5,5-tetramethyl-1,3,2-dioxaborolan-2-yl)benzo[d]oxazol-2-amine (2.04 g, 7.86 mmol, 1.2 equiv) and Na₂CO₃ (3.47 g, 32.73 mmol, 5.0 equiv) in DME (60 mL) and H₂O (30 mL) was added Pd(PPh₃)₄ (756.43 mg, 654.60 μmol, 0.1 equiv) at room temperature under N₂. The mixture was stirred at 110° C. for 3 h and the two batches were combined together. The reaction mixture was cooled and partitioned between EtOAc (500 mL) and H₂O (500 mL). The aqueous layer was separated and extracted with EtOAc (3×300 mL). All the organic layers were combined, washed with brine (20 mL), dried over anhydrous Na₂SO₄, filtered, and the filtrate was concentrated under reduced pressure to give tert-butyl 4-((4-amino-3-(2-aminobenzo[d]oxazol-5-yl)-1H-pyrazolo[3,4-d]pyrimidin-1-yl)methyl)piperidine-1-carboxylate (4.5 g, 74% yield) as a yellow solid. LCMS (ESI) m/z: [M+H] calcd for C₂₃H₂₈N₈O₃: 465.24; found 465.2.

### Step 3: Synthesis of 5-(4-amino-1-(piperidin-4-ylmethyl)-1H-pyrazolo[3,4-d]pyrimidin-3-yl)benzo[d]oxazol-2-amine

A solution of tert-butyl 4-((4-amino-3-(2-aminobenzo[d]oxazol-5-yl)-1H-pyrazolo[3,4-d]pyrimidin-1-yl)methyl)piperidine-1-carboxylate (2.5 g, 5.38 mmol, 1.0 equiv) in TFA (25 mL) was stirred at room temperature for 30 min. The reaction solution was concentrated under reduced pressure to remove TFA. The residue was added to MTBE (400 mL) and a solid precipitated, which was then filtered to give 5-(4-amino-1-(piperidin-4-ylmethyl)-1H-pyrazolo[3,4-d]pyrimidin-3-yl)benzo[d]oxazol-2-amine (2.7 g, over 100% yield, TFA) as a yellow solid. LCMS (ESI) m/z: [M+H] calcd for C₁₈H₂₀N₈O: 365.18; found 365.1.

### Monomer F. 2-(4-amino-1-(4-aminobutyl)-1H-pyrazolo[3,4-d]pyrimidin-3-yl)-1H-indol-5-ol trifluoroacetic acid salt

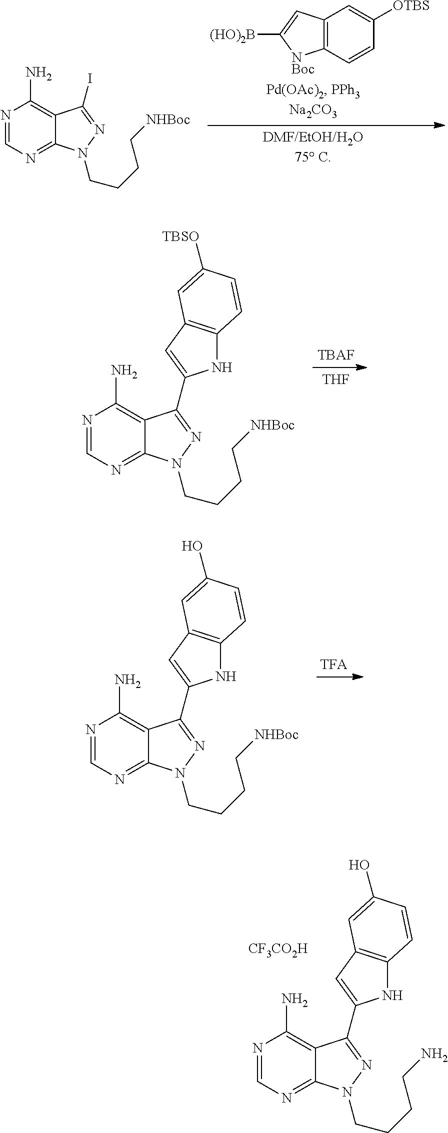

### Step 1: Synthesis of tert-butyl (4-(4-amino-3-(5-((tert-butyldimethylsilyl)oxy)-1H-indol-2-yl)-1H-pyrazolo[3,4-d]pyrimidin-1-yl)butyl)carbamate

To a solution of tert-butyl (4-(4-amino-3-iodo-1H-pyrazolo[3,4-d]pyrimidin-1-yl)butyl)carbamate (1.0 g, 2.31 mmol, 1.0 equiv) in dioxane (10.5 mL) and H₂O (3.5 mL) was added (1-(tert-butoxycarbonyl)-5-((tert-butyldimethylsilyl)oxy)-1H-indol-2-yl)boronic acid (1.54 g, 2.78 mmol, 1.2 equiv), K₃PO₄ (1.47 g, 6.94 mmol, 3.0 equiv), Pd₂(dba)₃ (211.84 mg, 231.34 μmol, 0.1 equiv), and SPhos (189.95 mg, 462.69 μmol, 0.2 equiv) at room temperature under N₂. The sealed tube was heated at 150° C. for 20 min in a microwave. This was repeated for 9 additional batches. The 10 batches were combined and the reaction mixture was cooled and partitioned between EtOAc (60 mL) and H₂O (80 mL). The aqueous layer was separated and extracted with EtOAc (2×50 mL). The organic layers were combined, washed with brine (60 mL) and dried over anhydrous Na₂SO₄. The suspension was filtered and the filtrate was concentrated under reduced pressure. The crude material was purified by silica gel chromatography (1→75% EtOAc/petroleum ether). The desired fractions were combined and evaporated under reduced pressure to give tert-butyl (4-(4-amino-3-(5-((tert-butyldimethylsilyl)oxy)-1H-indol-2-yl)-1H-pyrazolo[3,4-d]pyrimidin-1-yl)butyl)carbamate (10 g, 60% yield) as a light yellow solid.

### Step 2: Synthesis of tert-butyl (4-(4-amino-3-(5-hydroxy-1H-indol-2-yl)-1H-pyrazolo[3,4-d]pyrimidin-1-yl)butyl)carbamate

To a mixture of tert-butyl (4-(4-amino-3-(5-((tert-butyldimethylsilyl)oxy)-1H-indol-2-yl)-1H-pyrazolo[3,4-d]pyrimidin-1-yl)butyl)carbamate (10 g, 18.12 mmol, 1.0 equiv) in THF (100 mL) was added TBAF·3H₂O (1 M. 54.37 mL, 3.0 equiv) in one portion at room temperature under N₂. The mixture was stirred for 1 h and then H₂O (100 mL) was added to the reaction mixture. The layers were separated and the aqueous phase was extracted with EtOAc (2×80 mL). The combined organic phase was washed with brine (100 mL), dried with anhydrous Na₂SO₄, filtered and concentrated under reduced pressure. The residue was purified by silica gel chromatography (1→67% EtOAc/petroleum ether) to afford tert-butyl (4-(4-amino-3-(5-hydroxy-1H-indol-2-yl)-1H-pyrazolo[3,4-d]pyrimidin-1-yl)butyl)carbamate (7 g, 87% yield) as a light pink solid.

### Step 3: Synthesis of 2-[4-amino-1-(4-aminobutyl)pyrazolo[3,4-d]pyrimidin-3-yl]-1H-indol-5-ol

To TFA (50.0 mL, 675.26 mmol, 38.9 equiv) was added tert-butyl (4-(4-amino-3-(5-hydroxy-1H-indol-2-yl)-1H-pyrazolo[3,4-d]pyrimidin-1-yl)butyl)carbamate (7.6 g, 17.37 mmol, 1.0 equiv) at room temperature. The mixture was stirred for 40 min and was then concentrated under reduced pressure. The oily residue was triturated with MeCN (20 mL), then added dropwise into MTBE (300 mL) for 10 min. The supernatant was removed and then the precipitate was collected by filtration under N₂ to give 2-[4-amino-1-(4-aminobutyl)pyrazolo[3,4-d]pyrimidin-3-yl]-1H-indol-5-ol (7.79 g, 91% yield, TFA) as light yellow solid. LCMS (ESI) m/z: [M+H] calcd for C₁₇H₁₉N₇O: 338.17; found 338.2.

### Monomer G. 5-(4-amino-1-(azetidin-3-ylmethyl)-1H-pyrazolo[3,4-d]pyrimidin-3-yl)benzo[d]oxazol-2-amine trifluoroacetic acid salt

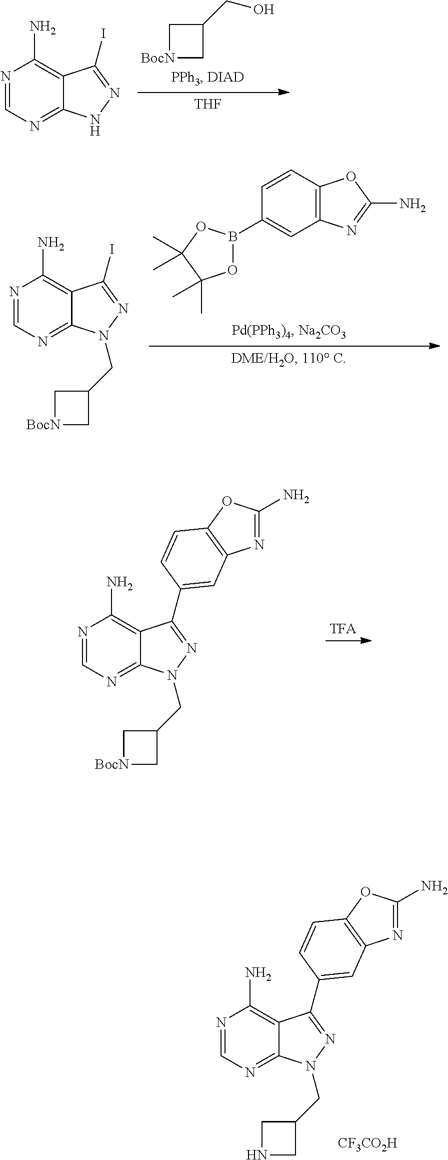

### Step 1: Synthesis of tert-butyl 3-((4-amino-3-iodo-1H-pyrazolo[3,4-d]pyrimidin-1-yl) methyl)azetidine-1-carboxylate

To a solution of 3-iodo-1H-pyrazolo[3,4-d]pyrimidin-4-amine (4 g, 15.32 mmol, 1.0 equiv), tert-butyl 3-(hydroxymethyl)azetidine-1-carboxylate (3.01 g, 16.09 mmol, 1.05 equiv) and PPh₃ (6.03 g, 22.99 mmol, 1.5 equiv) in THF (80 mL) cooled to 0° C. was added DIAD (4.47 mL, 22.99 mmol, 1.5 equiv), dropwise. After the addition was complete, the reaction was stirred at room temperature for 14 h. The reaction was poured into H₂O (200 mL) and then extracted with EtOAc (3×50 mL). The organic layers were combined and washed with brine (2×50 mL). The organic phase was dried over Na₂SO₄, filtered, the filtrate was concentrated under reduced pressure to give a residue. The residue was purified by silica gel chromatography (0→100% EtOAc/petroleum ether) to give tert-butyl 3-((4-amino-3-iodo-1H-pyrazolo[3,4-d]pyrimidin-1-yl)methyl) azetidine-1-carboxylate (4.2 g, 64% yield) as a white solid. LCMS (ESI) m/z: [M+H] calcd for C₁₄H₁₉IN₆O₂: 431.07; found 431.0.

### Step 2: Synthesis of tert-butyl 3-((4-amino-3-(2-aminobenzo[d]oxazol-5-yl)-1H-pyrazolo [3,4-d]pyrimidin-1-yl)methyl)azetidine-1-carboxylate

To a bi-phasic suspension of tert-butyl 3-((4-amino-3-iodo-1H-pyrazolo[3,4-d]pyrimidin-1-yl) methyl)azetidine-1-carboxylate (4 g, 9.30 mmol, 1.0 equiv), 5-(4,4,5,5-tetramethyl-1,3,2-dioxaborolan-2-yl)benzo[d]oxazol-2-amine (2.90 g, 11.16 mmol, 1.2 equiv) and Na₂CO₃ (4.93 g, 46.49 mmol, 5.0 equiv) in DME (100 mL) and H₂O (50 mL) was added Pd(PPh₃)₄ (1.07 g, 929.71 μmol, 0.1 equiv) at room temperature under N₂. The mixture was stirred at 110° C. for 3 h. The reaction mixture was then cooled to room temperature and filtered, and the filtrate was extracted by EtOAc (3×50 mL). The organic layers were combined and washed with brine (10 mL), dried over Na₂SO₄, filtered and the filtrate was concentrated under reduced pressure to give a residue. The residue was purified by silica gel chromatography (0→20% MeOH/EtOAc) to give tert-butyl 3-((4-amino-3-(2-aminobenzo[d]oxazol-5-yl)-1H-pyrazolo[3,4-d]pyrimidin-1-yl)methyl)azetidine-1-carboxylate (3.5 g, 80% yield) as a yellow solid. LCMS (ESI) m/z: [M+H] calcd for C₂₁H₂₄N₈O₃: 437.20; found 437.2.

### Step 3: Synthesis of 5-(4-amino-1-(azetidin-3-ylmethyl)-1H-pyrazolo[3,4-d]pyrimidin-3-yl)benzo[d]oxazol-2-amine

To a solution of tert-butyl 3-((4-amino-3-(2-aminobenzo[d]oxazol-5-yl)-1H-pyrazolo[3,4-d]pyrimidin-1-yl)methyl)azetidine-1-carboxylate (3.29 g, 6.87 mmol, 1.0 equiv) in DCM (20 mL) was added TFA (7.50 mL, 101.30 mmol, 14.7 equiv) at 0° C. The reaction was warmed to room temperature and stirred for 2 h. The reaction solution was concentrated under reduced pressure to give a residue. The residue was dissolved in MeCN (6 mL) and then poured into MTBE (80 mL). A solid precipitated, which was filtered and the solid cake was dried under reduced pressure to give 5-[4-amino-1-(azetidin-3-ylmethyl)pyrazolo[3,4-d]pyrimidin-3-yl]-1,3-benzoxazol-2-amine (4.34 g, over 100% yield, TFA) as a yellow solid. LCMS (ESI) m/z: [M+H] calcd for C₁₆H₁₆N₈O: 337.15; found 337.1.

### Monomer H. 5-(4-amino-1-(4-aminobutyl)-1H-pyrazolo[3,4-d]pyrimidin-3-yl)benzo[d]-oxazol-2-amine trifluoroacetic acid salt

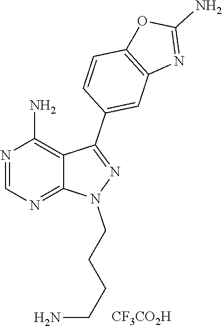 [SMILES: NCCCCn1nc(-c2ccc3oc(N)nc3c2)c2c(N)ncnc21 ; "CF3CO2H"]

This monomer was synthesized following the procedures outlined in Nature 2015, 534, 272-276, which is incorporated by reference in its entirety.

### Monomer I. 5-(4-amino-1-(pyrrolidin-3-ylmethyl)-1H-pyrazolo[3,4-d]pyrimidin-3-yl)benzo[d]oxazol-2-amine trifluoroacetic acid salt

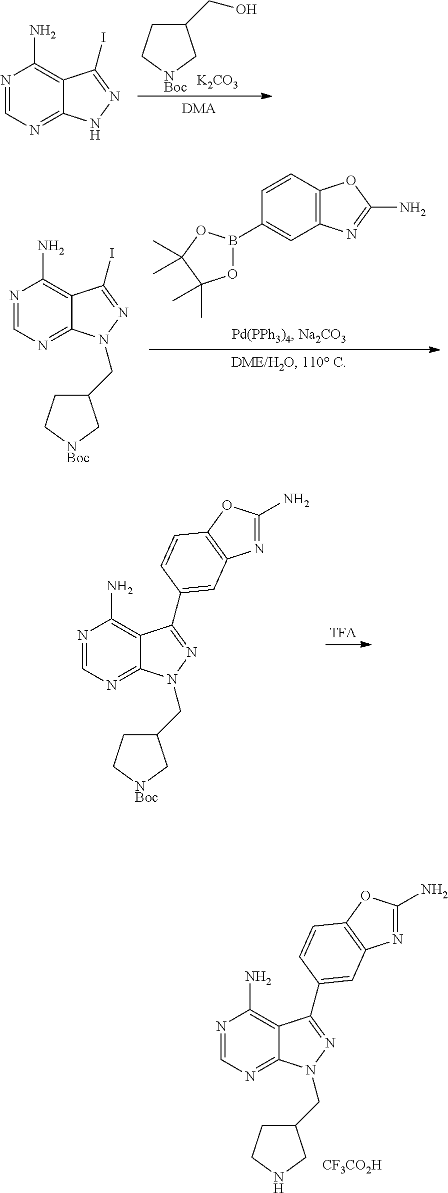

### Step 1: Synthesis of tert-butyl 3-((4-amino-3-iodo-1H-pyrazolo[3,4-d]pyrimidin-1-yl) methyl)pyrrolidine-1-carboxylate

A suspension of 3-iodo-1H-pyrazolo[3,4-d]pyrimidin-4-amine (4.5 g, 17.24 mmol, 1.0 equiv), tert-butyl 3-(bromomethyl)pyrrolidine-1-carboxylate (4.78 g, 18.10 mmol, 1.05 equiv) and K₂CO₃ (7.15 g, 51.72 mmol, 3.0 equiv) in DMA (40 mL) was heated to 85° C. The reaction was stirred at 85° C. for 3 h, at which point the solution was cooled to room temperature. Then. H₂O (80 mL) was added to the reaction, and a solid precipitated out. The mixture was filtered, and the solid cake was washed with H₂O (2×40 mL), and then dried under reduced pressure to give tert-butyl 3-((4-amino-3-iodo-1H-pyrazolo[3,4-d]pyrimidin-1-yl) methyl)pyrrolidine-1-carboxylate (6 g, 78% yield) as a yellow solid. LCMS (ESI) m/z: [M+H] calcd for C₁₅H₂₁IN₆O₂: 445.08; found 445.1.

### Step 2: Synthesis of tert-butyl 3-[[4-amino-3-(2-amino-1,3-benzoxazol-5-yl)pyrazolo[3,4-d]pyrimidin-1-yl]methyl]pyrrolidine-1-carboxylate

To a bi-phasic suspension of tert-butyl 3-((4-amino-3-iodo-1H-pyrazolo[3,4-d]pyrimidin-1-yl) methyl)pyrrolidine-1-carboxylate (4 g, 9.00 mmol, 1.0 equiv), 5-(4,4,5,5-tetramethyl-1,3,2-dioxaborolan-2-yl)benzo[d]oxazol-2-amine (2.81 g, 10.80 mmol, 1.2 equiv) and Na₂CO₃ (4.77 g, 45.02 mmol, 5.0 equiv) in DME (120 mL) and H₂O (60 mL) was added Pd(PPh₃)₄ (1.04 g, 900.35 μmol, 0.1 equiv) at room temperature under N₂. The mixture was stirred at 110° C. for 3 h. The reaction mixture was cooled to room temperature and filtered and the filtrate was extracted with EtOAc (3×50 mL). The organic phases were combined and washed with brine (50 mL), dried over Na₂SO₄, filtered and concentrated under reduced pressure to give a residue. The residue was purified by silica gel chromatography (0→20% MeOH/EtOAc) to give tert-butyl 3-((4-amino-3-(2-aminobenzo[d]oxazol-5-yl)-1H-pyrazolo[3,4-d]pyrimidin-1-yl) methyl)pyrrolidine-1-carboxylate (3 g, 64% yield) as a yellow solid. LCMS (ESI) m/z: [M+H] calcd for C₂₂H₂₆N₈O₃: 451.21, found 451.2.

### Step 3: Synthesis of 5-(4-amino-1-(pyrrolidin-3-ylmethyl)-1H-pyrazolo[3,4-d]pyrimidin-3-yl)benzo[d]oxazol-2-amine

To a solution of tert-butyl 3-((4-amino-3-(2-aminobenzo[d]oxazol-5-yl)-1H-pyrazolo[3,4-d]pyrimidin-1-yl)methyl)pyrrolidine-1-carboxylate (3 g, 6.66 mmol, 1.0 equiv) in DCM (40 mL) was added TFA (20 mL) at 0° C., dropwise. The reaction mixture was warmed to room temperature and stirred for 2 h. The reaction solution was then concentrated under reduced pressure to give a residue. The residue was dissolved in MeCN (4 mL), then poured into MTBE (100 mL), and a solid precipitated out. The solid was filtered and the cake was dried under reduced pressure to give 5-(4-amino-1-(pyrrolidin-3-ylmethyl)-1H-pyrazolo[3,4-d]pyrimidin-3-yl)benzo[d]oxazol-2-amine (4.00 g, over 100% yield, TFA) as a yellow solid. LCMS (ESI) m/z: [M+H] calcd for C₁₇H₁₈N₈O: 351.17; found 351.2.

### Monomer J. 1-(4-aminobutyl)-3-(7-methoxy-1H-indol-2-yl)-1H-pyrazolo[3,4-d]pyrimidin-4-aminetrifluoroacetic acid salt

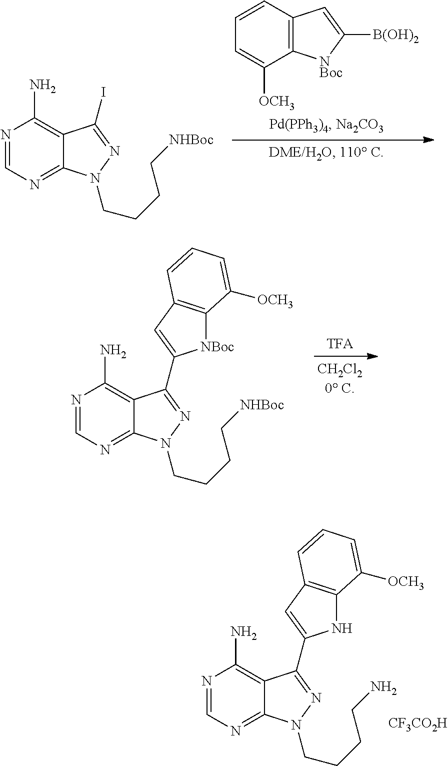 [SMILES: CC(C)(C)OC(=O)NCCCCn1nc(I)c2c(N)ncnc21 ; COc1cccc2cc(B(O)O)n(C(=O)OC(C)(C)C)c12 ; COc1cccc2cc(-c3nn(CCCCNC(=O)OC(C)(C)C)c4ncnc(N)c34)n(C(=O)OC(C)(C)C)c12 ; COc1cccc2cc(-c3nn(CCCCN)c4ncnc(N)c34)[nH]c12 ; "CF3CO2H"]

### Step 1: Synthesis of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate

To a mixture of tert-butyl (4-(4-amino-3-iodo-1H-pyrazolo[3,4-d]pyrimidin-1-yl)butyl)carbamate (1.0 equiv) and (1-(tert-butoxycarbonyl)-7-methoxy-1H-indol-2-yl)boronic acid (3.0 equiv) in DME and H₂O is added Pd(PPh₃)₄ (0.1 equiv) and sodium carbonate (6.0 equiv). The reaction is heated at 80° C. until completion, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 2: Synthesis of 1-(4-aminobutyl)-3-(7-methoxy-1H-indol-2-yl)-1H-pyrazolo[3,4-d]pyrimidin-4-amine

To a solution of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate (1.0 equiv) in DCM at 0° C. is added TFA dropwise. The reaction is stirred at 0° C. and warmed to room temperature. Once the reaction is complete, as determined by LCMS, the reaction is concentrated under reduced pressure. The residue is triturated with MeCN, then dripped into MTBE over 10 min. The supernatant is removed and the precipitate is collected by filtration under N₂ to give 1-(4-aminobutyl)-3-(7-methoxy-1H-indol-2-yl)-1H-pyrazolo[3,4-d]pyrimidin-4-amine.

### Monomer K. Synthesis of 1-(4-aminobutyl)-1H-pyrazolo[3,4-d]pyrimidin-4-amine trifluoroacetic acid salt

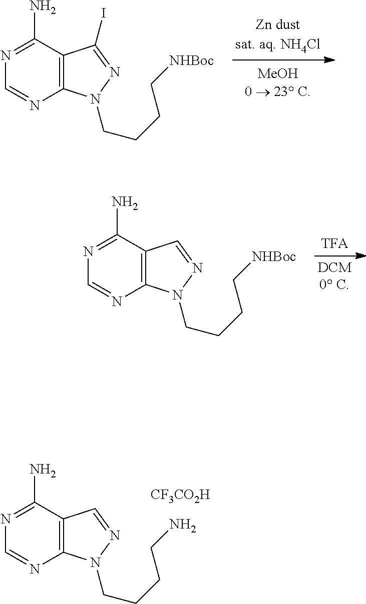 [SMILES: CC(C)(C)OC(=O)NCCCCn1nc(I)c2c(N)ncnc21 ; CC(C)(C)OC(=O)NCCCCn1ncc2c(N)ncnc21 ; NCCCCn1ncc2c(N)ncnc21 ; "CF3CO2H"]

### Step 1: Synthesis of tert-butyl (4-(4-amino-1H-pyrazolo[3,4-d]pyrimidin-1-yl)butyl)carbamate

To a mixture of tert-butyl (4-(4-amino-3-iodo-1H-pyrazolo[3,4-d]pyrimidin-1-yl)butyl)carbamate (300 mg, 694 μmol, 1.0 equiv) in MeOH (14 mL) at 0° C. was added zinc dust (226 mg, 3.46 mmol, 5.0 equiv). Sat, aq. NH₄Cl (14 mL) was added to the reaction mixture and the reaction was warmed to room temperature and stirred for 18 h. The reaction was quenched by EtOAc (40 mL) and H₂O (10 mL) and the mixture was transferred to a separatory funnel. The aqueous phase was extracted with EtOAc (3×20 mL) and the combined organic phases were washed with sat. aq. NaHCO₃ (15 mL), dried over Na₂SO₄, filtered, and concentrated under reduced pressure to provide the product (210 mg, 99% yield) as a light yellow solid that was used without further purification. LCMS (ESI) m/z: [M+H] calcd for C₁₄H₂₂N₆O₂: 307.19; found 307.1.

### Step 2: Synthesis of 1-(4-aminobutyl)-1H-pyrazolo[3,4-d]pyrimidin-4-amine

To a solution of tert-butyl (4-(4-amino-1H-pyrazolo[3,4-d]pyrimidin-1-yl)butyl)carbamate (210 mg, 691 mol) in DCM (3.5 mL) at 0° C. was added TFA (3.5 mL), dropwise. After 3 h, the reaction was warmed to room temperature and concentrated under reduced pressure to provide the trifluoroacetate salt of the product (220 mg, 99% yield) as a brown oil, which was used without further purification. LCMS (ESI) m/z: [M+H] calcd for C₉H₁₄N₆: 207.13; found 207.1.

### Monomer L. 1-[4-(piperazin-1-yl)-3-(trifluoromethyl)phenyl]-9-(quinolin-3-yl)-1H,2H-benzo[h]1,6-naphthyridin-2-one

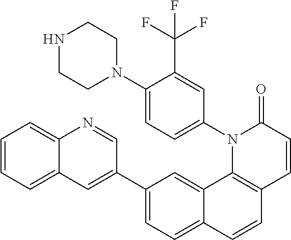 [SMILES: O=c1ccc2ccc3ccc(-c4cnc5ccccc5c4)cc3c2n1-c1ccc(N2CCNCC2)c(C(F)(F)F)c1]

The preparation of this monomer has been previously reported in the literature. See the following references: i) Liu, Qingsong; Chang, Jae Won; Wang, Jinhua; Kang, Seong A.; Thoreen, Carson C.; Markhard, Andrew; Hur, Wooyoung; Zhang, Jianming; Sim, Taebo; Sabatini, David M.; et al From Journal of Medicinal Chemistry (2010), 53(19), 7146-7155. ii) Gray, Nathanael; Chang, Jae Won; Zhang, Jianming; Thoreen, Carson C.; Kang, Seong Woo Anthony; Sabatini, David M.; Liu, Qingsong From PCT Int. Appl. (2010), WO 2010044885A2, which are incorporated by reference in their entirety.

### Monomer M. 5-(1-(4-aminobutyl)-4-(dimethylamino)-1H-pyrazolo[3,4-d]pyrimidin-3-yl)benzo[d]oxazol-2-amine trifluoroacetic acid salt

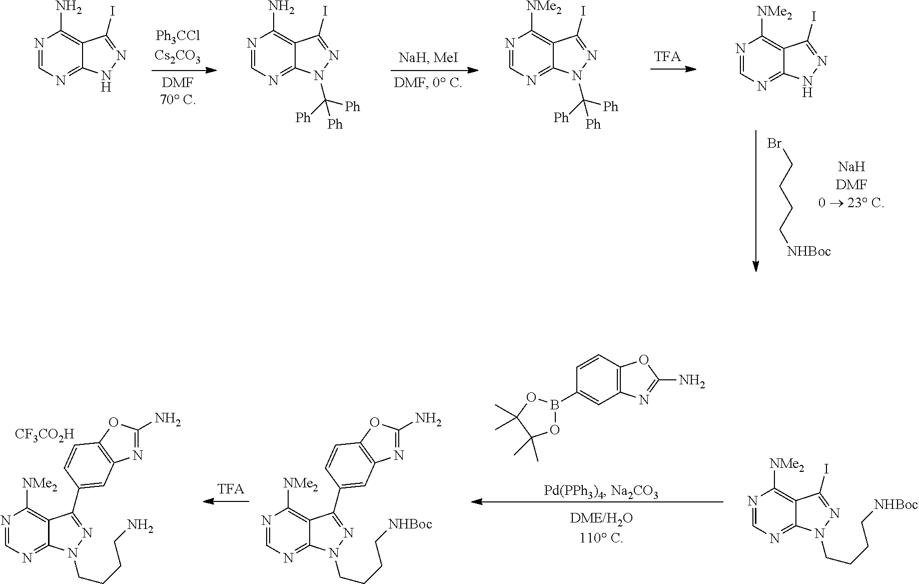

### Step 1: Synthesis of 3-iodo-1-trityl-1H-pyrazolo[3,4-d]pyrimidin-4-amine

A suspension of 3-iodo-1H-pyrazolo[3,4-d]pyrimidin-4-amine (10.5 g, 40.23 mmol, 1.0 equiv) in DMF (170.0 mL) was treated with Cs₂CO₃ (19.7 g, 60.34 mmol, 1.5 equiv) and [chloro(diphenyl)methyl]benzene (13.5 g, 48.27 mmol, 1.2 equiv) at room temperature. The reaction mixture was stirred at 70° C. for 4 h under a nitrogen atmosphere. The reaction mixture was added to H₂O (1200 mL). The precipitate was filtered and washed with H₂O. The residue was purified by silica gel chromatography (0→60% EtOAc/petroleum ether) to afford 3-iodo-1-trityl-1H-pyrazolo[3,4-d]pyrimidin-4-amine (15.40 g, 73.5% yield) as a white solid.

### Step 2: Synthesis of 3-iodo-N,N-dimethyl-1-trityl-1H-pyrazolo[3,4-d]pyrimidin-4-amine

To a suspension of NaH (2.98 g, 74.50 mmol, 60 wt. %, 2.5 equiv) in DMF (150 mL) was added the solution of 3-iodo-1-trityl-1H-pyrazolo[3,4-d]pyrimidin-4-amine (15.0 g, 29.80 mmol, 1.0 equiv) in DMF (50 mL) at 0° C. The mixture was stirred at 0° C. for 10 min. To the reaction mixture was then added iodomethane (16.92 g, 119.20 mmol, 7.42 mL, 4.0 equiv) at 0° C. The mixture was stirred at room temperature for 2 h, at which point H₂O (1400 mL) was added at 0° C. The mixture was stirred for an additional 10 min at 0° C. The resulting precipitate was collected by filtration to give crude product, which was purified by silica gel chromatography (1%→25% EtOAc/petroleum ether) twice to afford 3-iodo-N,N-dimethyl-1-trityl-1H-pyrazolo[3,4-d]pyrimidin-4-amine (9.0 g, 89% yield) as a white solid.

### Step 3: Synthesis of 3-iodo-N,N-dimethyl-1H-pyrazolo[3,4-d]pyrimidin-4-amine

To a cooled solution of TFA (19.1 mL, 258.1 mmol, 15.0 equiv) in DCM (100.0 mL) was added 3-iodo-N,N-dimethyl-1-trityl-1H-pyrazolo[3,4-d]pyrimidin-4-amine (9.10 g, 17.12 mmol, 1.0 equiv) at 4° C. The mixture was stirred at room temperature for 1 h. The residue was poured into H₂O (100 mL) and the aqueous phase was extracted with DCM (2×50 mL). To the aqueous phase was then added a saturated aqueous solution of NaHCO₃ until the solution was pH 8. The resulting precipitate was collected by filtration to give 3-iodo-N,N-dimethyl-1H-pyrazolo[3,4-d]pyrimidin-4-amine (3.40 g, 68.7% yield) as a white solid.

### Step 4: Synthesis of tert-butyl (4-(4-(dimethylamino)-3-iodo-1H-pyrazolo[3,4-d]pyrimidin-1-yl)butyl)carbamate

To a suspension of 3-iodo-N,N-dimethyl-1H-pyrazolo[3,4-d]pyrimidin-4-amine (1.7 g, 5.88 mmol, 1.0 equiv) in DMF (20 mL) was added NaH (247 mg, 6.17 mmol, 60 wt. %, 1.05 equiv) at 4° C. The mixture was stirred at 4° C. for 30 min. To the reaction mixture was then added tert-butyl N-(4-bromobutyl)carbamate (2.22 g, 8.82 mmol, 1.81 mL. 1.5 equiv) in DMF (10 mL) at 4° C. The mixture was stirred at room temperature for 2 h. To the mixture was then added H₂O (100 mL) at 4° C. The mixture was stirred for an additional 30 min at 4° C. and the resulting precipitate was collected by filtration to give crude product. The residue was purified by silica gel chromatography (0→75% EtOAc/petroleum ether) to afford tert-butyl(4-(4-(dimethylamino)-3-iodo-1H-pyrazolo[3,4-d]pyrimidin-1-yl)butyl)carbamate (2.0 g, 56% yield) as a white solid.

### Step 5: Synthesis of tert-butyl (4-(3-(2-aminobenzo[d]oxazol-5-yl)-4-(dimethylamino)-1H-pyrazolo[3,4-d]pyrimidin-1-yl)butyl)carbamate

To a bi-phasic suspension of tert-butyl (4-(4-(dimethylamino)-3-iodo-1H-pyrazolo[3,4-d]pyrimidin-1-yl)butyl)carbamate (4.0 g, 8.69 mmol, 1.0 equiv), 5-(4,4,5,5-tetramethyl-1,3,2-dioxaborolan-2-yl)benzo[d]oxazol-2-amine (3.4 g, 13.03 mmol, 1.5 equiv), and Na₂CO₃ (4.6 g, 43.45 mmol, 5.0 equiv) in DME (80.0 mL) and H₂O (40.0 mL) was added Pd(PPh₃)₄ (1.0 g, 868.98 μmol, 0.1 equiv) at room temperature under N₂. The mixture was stirred at 110° C. for 3 h. The reaction mixture was then cooled and partitioned between EtOAc (300 mL) and H₂O (600 mL). The aqueous layer was separated and extracted with EtOAc (2×100 mL). The organic layers were combined, washed with brine (2×60 mL) and dried over anhydrous Na₂SO₄, filtered, and concentrated under reduced pressure. The crude material was purified by silica gel column chromatography (50% EtOAc/hexanes followed by 20% MeOH/EtOAc). The desired fractions were combined and concentrated under reduced pressure to give tert-butyl (4-(3-(2-aminobenzo[d]oxazol-5-yl)-4-(dimethylamino)-1H-pyrazolo[3,4-d]pyramidin-1-yl)butyl)carbamate (3.2 g, 78.9% yield) as a light brown solid.

### Step 6: Synthesis of 5-(1-(4-aminobutyl)-4-(dimethylamino)-1H-pyrazolo[3,4-d]pyrimidin-3-yl)benzo[d]oxazol-2-amine

To TFA (20.82 mL, 281.27 mmol, 36.5 equiv) was added tert-butyl (4-(3-(2-aminobenzo[d]oxazol-5-yl)-4-(dimethylamino)-1H-pyrazolo[3,4-d]pyrimidin-1-yl)butyl)carbamate (3.6 g, 7.72 mmol, 1.0 equiv) at room temperature. The mixture was stirred for 30 min, at which point the mixture was concentrated under reduced pressure. The oily residue was triturated with MeCN (8 mL) and MTBE (60 mL) for 10 min. The supernatant was removed and then the precipitate was collected by filtration under N₂ to give 5-(1-(4-aminobutyl)-4-(dimethylamino)-1H-pyrazolo[3,4-d]pyrimidin-3-yl)benzo[d]oxazol-2-amine (4.0 g, crude, TFA) as a light brown solid.

To 1 M NaOH (107.2 mL, 14.7 equiv) was added 5-(1-(4-aminobutyl)-4-(dimethylamino)-1H-pyrazolo[3,4-d]pyrimidin-3-yl)benzo[d]oxazol-2-amine (3.5 g, crude, TFA) at room temperature. The mixture was stirred for 10 min and then the aqueous phase was extracted with DCM (3×50 mL). The combined organic phase was washed with brine (50 mL), dried with anhydrous Na₂SO₄, filtered and concentrated under reduced pressure. TFA (539.37 μL, 7.28 mmol, 1.0 equiv) was added and concentrated under reduced pressure. MeCN (10 mL) was then added, followed by MTBE (150 mL). The resulting precipitate was collected by filtration to give 5-(1-(4-aminobutyl)-4-(dimethylamino)-1H-pyrazolo[3,4-d]pyrimidin-3-yl)benzo[d]oxazol-2-amine (1.3 g, 36.6% yield, TFA) as a light brown product. LCMS (ESI) m/z: [M+H] calcd for C₁₈H₂₂N₈O: 367.19; found 367.1.

### Monomer N. 6-(4-amino-1-(4-aminobutyl)-1H-pyrazolo[3,4-d]pyrimidin-3-yl)benzo-[d]isoxazol-3-amine trifluoroacetic acid salt

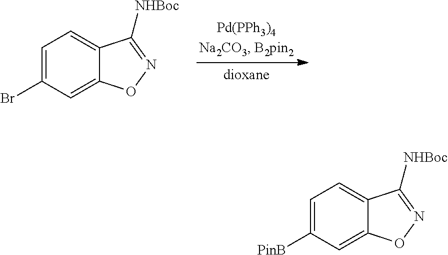

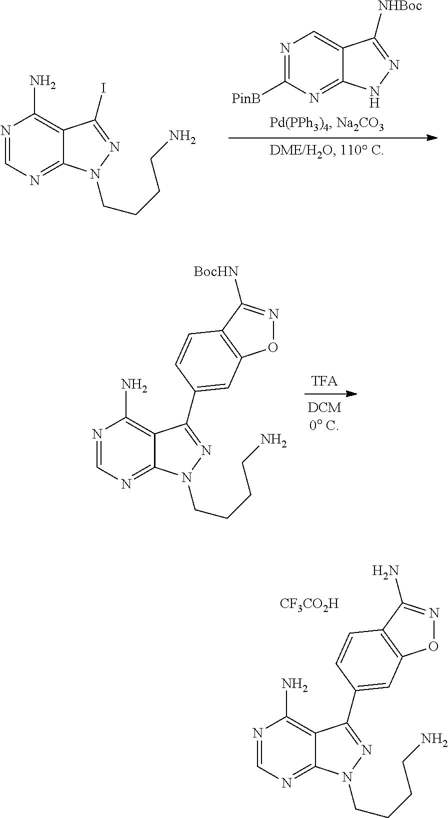

### Step 1: Synthesis of tert-butyl (6-(4,4,5,5-tetramethyl-1,3,2-dioxaborolan-2-yl)benzo[d]isoxazol-3-yl)carbamate

To a solution of tert-butyl (6-bromobenzo[d]isoxazol-3-yl)carbamate (1.0 equiv) in dioxane is added Pd(PPh₃)₄ (0.1 equiv), sodium carbonate (6.0 equiv), and bis(pinacolato)diboron (3.0 equiv). The reaction mixture is stirred and heated until completion, as determined by LCMS and TLC analysis. The reaction is cooled to room temperature, quenched with sat. aq. NaHCO₃, and the mixture transferred to a separatory funnel. The aqueous phase is extracted with EtOAc and the organic phase is washed with sat. aq. NaCl, dried over Na₂SO₄, filtered, and concentrated under reduced pressure. The desired product is isolated after purification by silica gel chromatography.

### Step 2: Synthesis of tert-butyl (4-(4-amino-3-(3-((tert-butoxycarbonyl)amino)benzo[d]isoxazol-6-yl)-1H-pyrazolo[3,4-d]pyrimidin-1-yl)butyl)carbamate

To a mixture of tert-butyl (4-(4-amino-3-iodo-1H-pyrazolo[3,4-d]pyrimidin-1-yl)butyl)carbamate (1.0 equiv) and tert-butyl (6-(4,4,5,5-tetramethyl-1,3,2-dioxaborolan-2-yl)benzo[d]isoxazol-3-yl)carbamate (3.0 equiv) in DME and H₂O is added Pd(PPh₃)₄ (0.1 equiv) and sodium carbonate (6.0 equiv). The reaction is heated at 80° C. until completion, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 3: Synthesis of 6-(4-amino-1-(4-aminobutyl)-1H-pyrazolo[3,4-d]pyrimidin-3-yl)benzo-[d]isoxazol-3-amine

To a solution of tert-butyl (4-(4-amino-3-(3-((tert-butoxycarbonyl)amino)benzo[d]isoxazol-6-yl)-1H-pyrazolo[3,4-d]pyrimidin-1-yl)butyl)carbamate (1.0 equiv) in DCM at 0° C. is added TFA, dropwise. The reaction is stirred at 0° C. and warmed to room temperature. Once the reaction is complete, as determined by LCMS, the reaction is concentrated under reduced pressure. The residue is triturated with MeCN, then added dropwise into MTBE over 10 min. The supernatant is removed and the precipitate is collected by filtration under N₂ to give 6-(4-amino-1-(4-aminobutyl)-1H-pyrazolo[3,4-d]pyrimidin-3-yl)benzo-[d]isoxazol-3-amine.

### Monomer O, 4-(5-(4-morpholino-1-(1-(pyridin-3-ylmethyl)piperidin-4-yl)-1H-pyrazolo[3,4-d]pyrimidin-6-yl)-1H-indol-1-yl)butan-1-amine trifluoroacetic acid salt

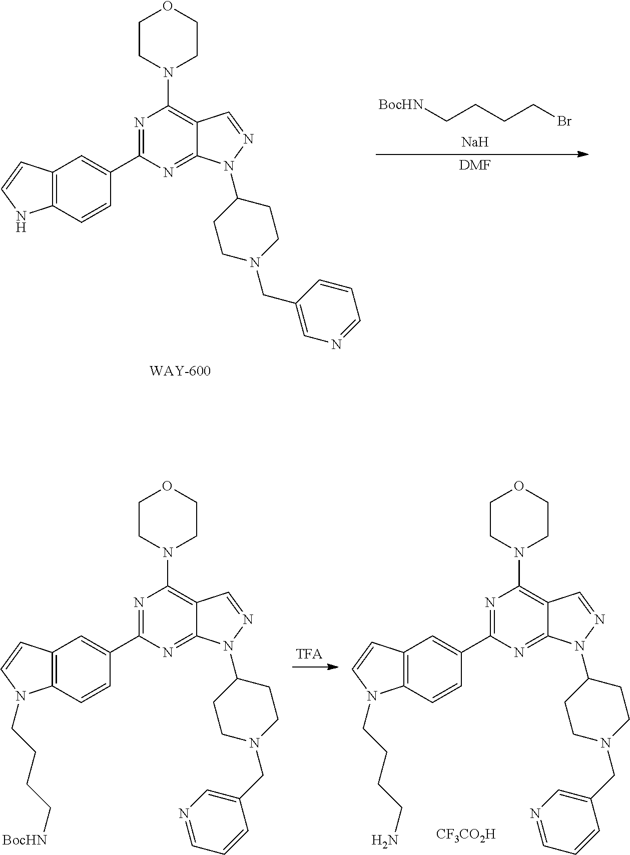

The synthesis of this monomer proceeds by alkylation of WAY-600 (CAS #1062159-35-6) with tert-butyl (4-bromobutyl)carbamate under basic conditions, followed by Boc-deprotection using TFA to produce the TFA salt.

Reference for preparation of WAY-600: Discovery of Potent and Selective Inhibitors of the Mammalian Target of Rapamycin (mTOR) Kinase: Nowak. P.; Cole, D. C.; Brooijmans, N.; Bursavich, M. G.; Curran, K. J.; Ellingboe, J. W.; Gibbons, J. J.; Hollander, I.; Hu, Y.; Kaplan, J.; Malwitz, D. J.; Toral-Barza, L.; Verheijen, J. C.; Zask, A.; Zhang, W.-G.; Yu, K. 2009; Journal of Medicinal Chemistry Volume 52, Issue 22, 7081-89, which is incorporated by reference in its entirety.

### Monomer P. 2-(4-(8-(6-(aminomethyl)quinolin-3-yl)-3-methyl-2-oxo-2,3-dihydro-1H-imidazo[4,5-c]quinolin-1-yl)phenyl)-2-methylpropanenitrile trifluoroacetic acid salt

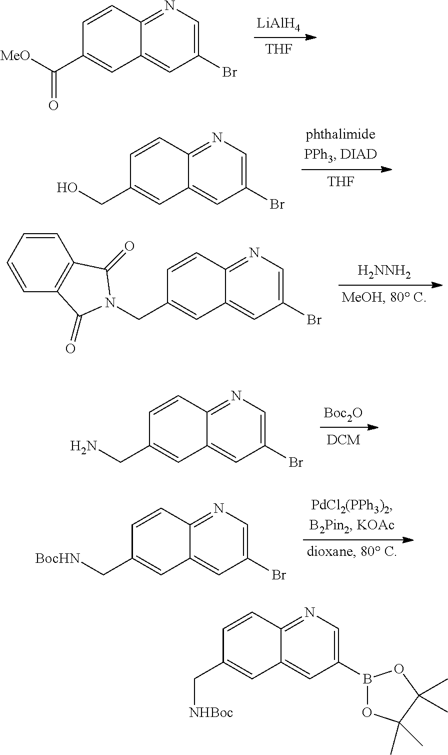

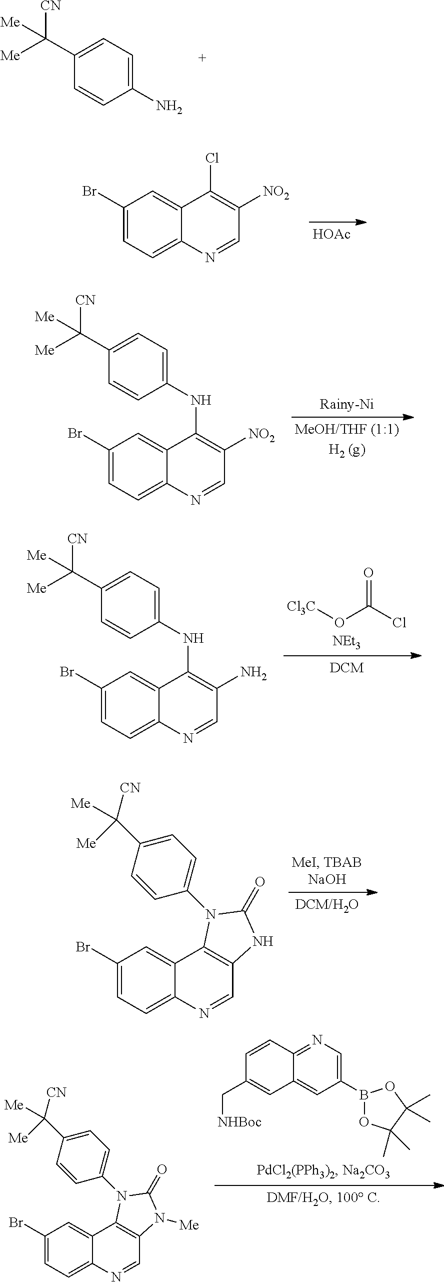

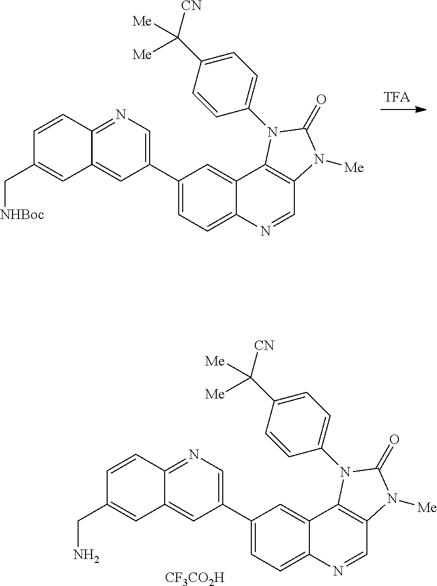 [SMILES: Cn1c(=O)n(-c2ccc(C(C)(C)C#N)cc2)c2c3cc(-c4cnc5ccc(CNC(=O)OC(C)(C)C)cc5c4)ccc3ncc21 ; Cn1c(=O)n(-c2ccc(C(C)(C)C#N)cc2)c2c3cc(-c4cnc5ccc(CN)cc5c4)ccc3ncc21 ; "CF3CO2H"]

The synthesis of this monomer proceeds first by synthesis of the Suzuki reaction coupling partner (3-(4,4,5,5-tetramethyl-1,3,2-dioxaborolane)quinolin-6-yl)-N-boc-methanamine starting from methyl 3-bromoquinoline-6-carboxylate. Reduction of the methyl ester with lithium aluminum hydride followed by Mitsunobu reaction with phthalimide and hydrazine cleavage provides the benzylic amine. Protection of the benzylic amine with di-tert-butyl dicarbonate followed by a Miyaura borylation reaction provides (3-(4,4,5,5-tetramethyl-1,3,2-dioxaborolane)quinolin-6-yl)-N-boc-methanamine.

An S_{N}Ar reaction of 2-(4-aminophenyl)-2-methylpropanenitrile with 6-bromo-4-chloro-3-nitroquinoline provides the substituted amino-nitro-pyridine. Reduction of the nitro group with Raney-Ni under a hydrogen atmosphere followed by cyclization with trichloromethyl chloroformate provides the aryl-substituted urea. Substitution of the free N—H of the urea with methyl iodide mediated by tetrabutylammonium bromide and sodium hydroxide followed by Suzuki coupling of (3-(4,4,5,5-tetramethyl-1,3,2-dioxaborolane)quinolin-6-yl)-N-boc-methanamine and then Boc-deprotection using TFA produces the TFA salt.

Reference for preparation of 2-[4-(8-bromo-3-methyl-2-oxo-2,3-dihydro-imidazo [4,5-c]quinolin-1-yl)-phenyl]-2-methyl-propionitrile: Vannucchi, A. M.; Bogani, C.; Bartalucci, N. 2016. JAK PI3K/mTOR combination therapy. U.S. Pat. No. 9,358,229. Novartis Pharma AG, Incyte Corporation, which is incorporated by reference in its entirety.

### Monomer Q. 8-(6-methoxypyridin-3-yl)-3-methyl-1-[4-(piperazin-1-yl)-3-(trifluoromethyl)phenyl]-1H,2H,3H-imidazo[4,5-c]quinolin-2-one

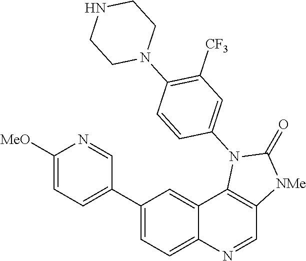 [SMILES: COc1ccc(-c2ccc3ncc4c(c3c2)n(-c2ccc(N3CCNCC3)c(C(F)(F)F)c2)c(=O)n4C)cn1]

This monomer is a commercially available chemical known as BGT226 (CAS #1245537-68-1). At the time this application was prepared, it was available for purchase from several vendors as the free amine.

### Monomer R. 3-(4-amino-1-(4-aminobutyl)-1H-pyrazolo[3,4-d]pyrimidin-3-yl)-N-(4,5-dihydrothiazol-2-yl)benzamide trifluoroacetic acid salt

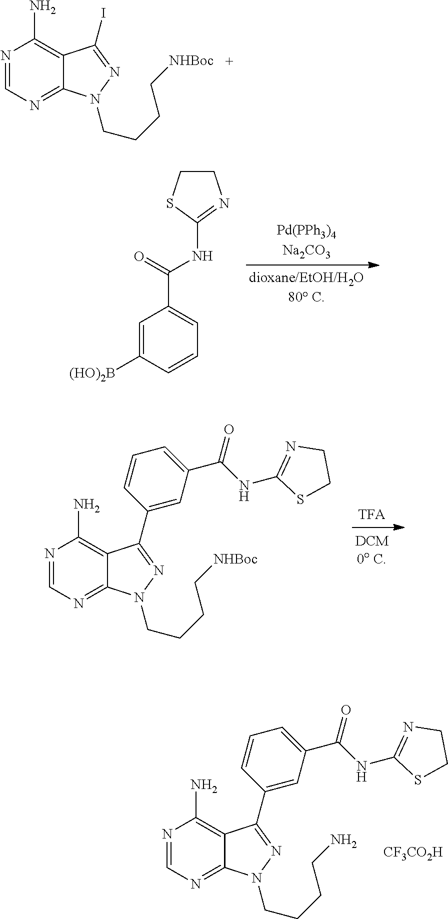 [SMILES: CC(C)(C)OC(=O)NCCCCn1nc(I)c2c(N)ncnc21 ; O=C(NC1=NCCS1)c1cccc(B(O)O)c1 ; CC(C)(C)OC(=O)NCCCCn1nc(-c2cccc(C(=O)NC3=NCCS3)c2)c2c(N)ncnc21 ; NCCCCn1nc(-c2cccc(C(=O)NC3=NCCS3)c2)c2c(N)ncnc21 ; "CF3CO2H"]

### Step 1: Synthesis of tert-butyl (4-(4-amino-3-(3-((4,5-dihydrothiazol-2-yl)carbamoyl)phenyl)-1H-pyrazolo[3,4-d]pyrimidin-1-yl)butyl)carbamate

To a solution of (3-((4,5-dihydrothiazol-2-yl)carbamoyl)phenyl)boronic acid (500 mg, 1.15 mmol, 1.0 equiv) and tert-butyl (4-(4-amino-3-iodo-1H-pyrazolo[3,4-d]pyrimidin-1-yl)butyl)carbamate (575 mg, 2.30 mmol, 2.0 equiv) in dioxane (19.1 mL), EtOH (3.8 mL), and H₂O (2.3 mL) was added Pd(PPh₃)₄ (265 mg, 230 μmol, 0.2 equiv) and sodium carbonate (730 mg, 6.89 mmol, 6.0 equiv). The reaction mixture was sonicated until formation of a clear, yellow solution, which was subsequently heated at 80° C. for 14 h. The reaction was then diluted with sat. aq. NaCl (30 mL) and the mixture transferred to a separatory funnel. The aqueous phase was extracted with DCM (3×25 mL). The combined organic phases were dried over Na₂SO₄, filtered, and concentrated under reduced pressure. The desired product was isolated as a yellow solid (324 mg, 53% yield) after silica gel chromatography (0→15% MeOH/DCM). LCMS (ESI) m/z: [M+H] calcd for C₂₄H₃₀N₈O₃S: 511.22; found 511.2.

### Step 2: Synthesis of 3-(4-amino-1-(4-aminobutyl)-1H-pyrazolo[3,4-d]pyrimidin-3-yl)-N-(4,5-dihydrothiazol-2-yl)benzamide

To a solution of tert-butyl (4-(4-amino-3-(3-((4,5-dihydrothiazol-2-yl)carbamoyl)phenyl)-1H-pyrazolo[3,4-d]pyrimidin-1-yl)butyl)carbamate (324 mg, 614 μmol) in DCM (4.1 mL) at 0° C. was added TFA (1.5 mL), dropwise. After 1 h, the reaction was warmed to room temperature and concentrated under reduced pressure to provide the trifluoroacetate salt of the product as a yellow solid (320 mg, 99% yield). Used without further purification. LCMS (ESI) m/z: [M+H] calcd for C₁₉H₂₂N₈OS: 411.16; found 411.1.

### Monomer S. 2-(5-(4-morpholino-1-(1-(pyridin-3-ylmethyl)piperidin-4-yl)-1H-pyrazolo[3,4-d]pyrimidin-6-yl)-1H-indol-3-yl)ethan-1-amine

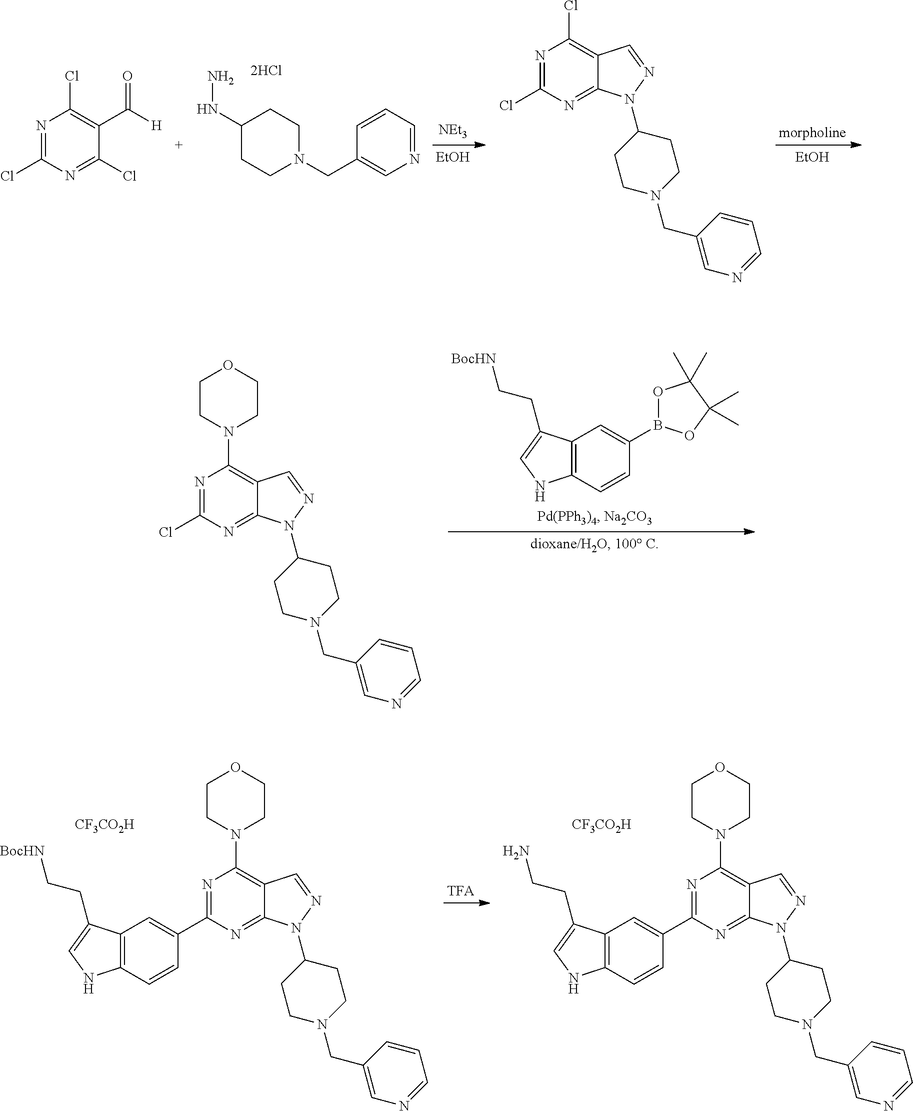

The synthesis of this monomer proceeds by condensation of 2,4,6-trichloropyrimidine-5-carbaldehyde with 3-((4-hydrazineylpiperidin-1-yl)methyl)pyridine hydrochloride. Reaction of the product with morpholine followed by a Suzuki reaction with boronic ester gives the Boc-protected amine. Final deprotection with TFA gives the monomer. This synthesis route follows closely to the reported preparation of highly related structures in the following references: i) Nowak, Pawel; Cole, Derek C.; Brooijmans, Natasja; Curran, Kevin J.; Ellingboe, John W.; Gibbons, James J.; Hollander, Irwin; Hu, Yong Bo; Kaplan, Joshua; Malwitz, David J.; et al From Journal of Medicinal Chemistry (2009), 52(22), 7081-7089. ii) Zask, Arie; Nowak, Pawel Wojciech; Verheijen, Jeroen; Curran, Kevin J.; Kaplan, Joshua; Malwitz, David; Bursavich, Matthew Gregory; Cole, Derek Cecil; Ayral-Kaloustian, Semiramis; Yu, Ker; et al From PCT Int. Appl. (2008), WO 2008115974 A2 20080925, which are incorporated by reference in their entirety.

### Monomer T. 1-(4-aminobutyl)-3-iodo-1H-pyrazolo[3,4-d]pyrimidin-4-amine trifluoroacetic acid salt

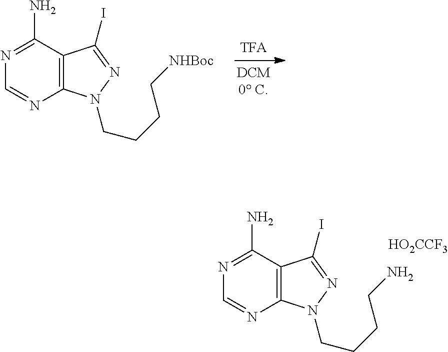

To a mixture of tert-butyl (4-(4-amino-3-iodo-1H-pyrazolo[3,4-d]pyrimidin-1-yl)butyl)carbamate (496 mg, 1.14 mmol, 1.0 equiv) in DCM (5.7 mL) at 0° C. was added TFA (1.5 mL) dropwise. The reaction was allowed to stir at 0° C. for 1 h, at which time the reaction was concentrated under reduced pressure to provide a yellow solid (505 mg, 99% yield) which was taken on without further purification. LCMS (ESI) m/z: [M+H] calcd for C₉H₁₃IN₆: 333.02; found 332.9.

### Monomer U. 5-(4-amino-1-(4-(methylamino)butyl)-1H-pyrazolo[3,4-d]pyrimidin-3-yl)benzo[d]oxazol-2-amine trifluoroacetic acid salt

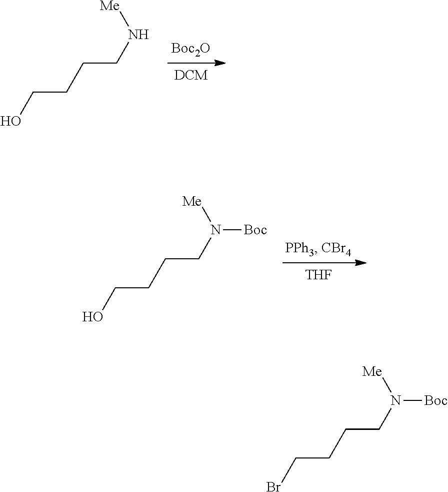

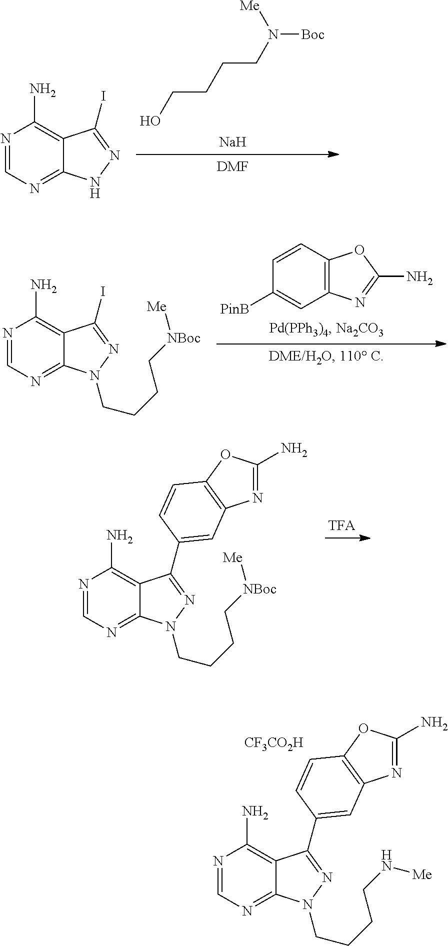

### Step 1: Synthesis of tert-butyl (4-hydroxybutyl)(methyl)carbamate

To a solution of 4-(methylamino)butan-1-ol (0.5 g, 4.85 mmol, 104.2 mL, 1.0 equiv) in DCM (10 mL) at room temperature was added Boc₂O (1.06 g, 4.85 mmol, 1.11 mL, 1.0 equiv). The mixture was stirred for 3 h at room temperature and then the mixture was concentrated under reduced pressure at 30° C. The residue was purified by silica gel chromatography (100/1 to 3/1 petroleum ether/EtOAc) to afford tert-butyl (4-hydroxybutyl)(methyl)carbamate (0.9 g, 91.4% yield) as a colorless oil.

### Step 2: Synthesis of tert-butyl (4-bromobutyl)(methyl)carbamate

To a solution of tert-butyl (4-hydroxybutyl)(methyl)carbamate (0.9 g, 4.43 mmol, 1.0 equiv) in THF (20 mL) at room temperature was added PPh₃ (2.21 g, 8.41 mmol, 1.9 equiv) and CBr₄ (2.79 g, 8.41 mmol, 1.9 equiv). The mixture was stirred for 1 h and then the reaction mixture was filtered and concentrated. The residue was purified by silica gel chromatography (1/0 to 4/1 petroleum ether/EtOAc) to afford tert-butyl (4-bromobutyl)(methyl) carbamate (1.1 g, 93.3% yield) as a colorless oil.

### Step 3: Synthesis of tert-butyl (4-(4-amino-3-iodo-1H-pyrazolo[3,4-d]pyrimidin-1-yl) butyl) (methyl)carbamate

To a suspension of 3-iodo-1H-pyrazolo[3,4-d]pyrimidin-4-amine (0.9 g, 3.45 mmol, 1.0 equiv) in DMF (10 mL) at 4° C. was added NaH (137.92 mg, 3.45 mmol, 60 wt. %, 1.0 equiv). The mixture was stirred at 4° C. for 30 min and then a solution of tert-butyl (4-bromobutyl)(methyl)carbamate (1.01 g, 3.79 mmol, 25.92 mL, 1.1 equiv) in DMF (3 mL) was added. The mixture was stirred at room temperature for 3 h, at which point H₂O (100 mL) was added. The aqueous phase was extracted with EtOAc (3×30 mL) and the combined organic phases were washed with brine (20 mL), dried with anhydrous Na₂SO₄, filtered and concentrated under reduced pressure. The residue was purified by silica gel chromatography (1/0 to 0/1 petroleum ether/EtOAc) to afford tert-butyl (4-(4-amino-3-iodo-1H-pyrazolo[3,4-d]pyrimidin-1-yl)butyl) (methyl) carbamate (1.2 g, 78% yield) as a white solid. LCMS (ESI) m/z: [M+H] calcd for C₁₅H₂₃IN₆O₂: 447.10; found 447.1.

### Step 4: Synthesis of tert-butyl (4-(4-amino-3-(2-aminobenzo[d]oxazol-5-yl)-1H-pyrazolo[3,4-d] pyrimidin-1-yl)butyl)(methyl)carbamate

To a bi-phasic suspension of tert-butyl (4-(4-amino-3-iodo-1H-pyrazolo[3,4-d]pyrimidin-1-yl)butyl)(methyl)carbamate (1.2 g, 2.69 mmol, 1.0 equiv), 5-(4,4,5,5-tetramethyl-1,3,2-dioxaborolan-2-yl)benzo[d]oxazol-2-amine (1.19 g, 3.23 mmol, 1.2 equiv), and Na₂CO₃ (1.42 g, 13.44 mmol, 5.0 equiv) in DME (20 mL) and H₂O (10 mL) at room temperature was added Pd(PPh₃)₄ (310.71 mg, 268.89 μmol, 0.1 equiv) under N₂. The mixture was stirred at 110° C. for 3 h and then the reaction mixture was cooled and partitioned between EtOAc (20 mL) and H₂O (15 mL). The aqueous layer was separated and extracted with EtOAc (3×20 mL). The combined organic layers were washed with brine (2×20 mL), dried over anhydrous Na₂SO₄, filtered and concentrated under reduced pressure. The crude product was purified by silica gel chromatography (1/0 to 4/1 EtOAc/MeOH) to give tert-butyl (4-(4-amino-3-(2-aminobenzo[d]oxazol-5-yl)-1H-pyrazolo[3,4-d]pyrimidin-1-yl)butyl)(methyl) carbamate (0.78 g, 62.5% yield) as an orange solid.

### Step 5: Synthesis of 5-(4-amino-1-(4-(methylamino)butyl)-1H-pyrazolo[3,4-d]pyrimidin-3-yl) benzo[d]oxazol-2-amine

A solution of tert-butyl(4-(4-amino-3-(2-aminobenzo[d]oxazol-5-yl)-1H-pyrazolo[3,4-d]pyrimidin-1-yl)butyl)(methyl)carbamate (0.78 g, 1.72 mmol, 1.0 equiv) in TFA (5 mL) at room temperature was stirred for 30 min. The solution was concentrated under reduced pressure and the oily residue was triturated with MeCN (1 mL) and then added to MTBE (100 mL). The supernatant was removed and then the precipitate was collected by filtration under N₂ to give 5-(4-amino-1-(4-(methylamino) butyl)-1H-pyrazolo[3,4-d]pyrimidin-3-yl)benzo[d]oxazol-2-amine bis-trifluorosulfonate (0.959 g, 93% yield) as an orange solid. LCMS (ESI) m/z: [M+H] calcd for C₁₇H₂₀N₈O: 353.18; found 353.1.

### Monomer V. 1-(4-(4-(5-(aminomethyl)pyrimidin-2-yl)piperazin-1-yl)-3-(trifluoromethyl)phenyl)-8-(6-methoxypyridin-3-yl)-3-methyl-1,3-dihydro-2H-imidazo[4,5-c]quinolin-2-one

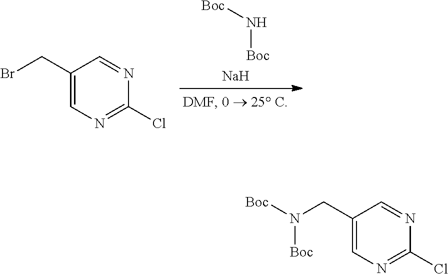

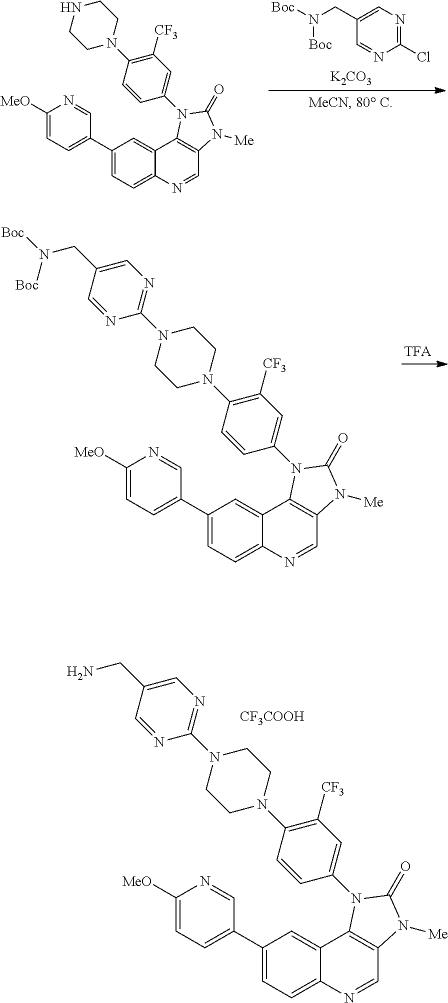

### Step 1: Synthesis of tert-butyl N-tert-butoxycarbonyl-N-[(2-chloropyrimidin-5-yl)methyl]carbamate

To a solution of tert-butyl N-tert-butoxycarbonylcarbamate (7.33 g, 33.74 mmol, 1.0 equiv) in DMF (80 mL) was added NaH (1.62 g, 40.49 mmol, 60 wt. %, 1.2 equiv) at 0° C. The mixture was stirred at 0° C. for 30 min and then 5-(bromomethyl)-2-chloro-pyrimidine (7 g, 33.74 mmol, 1 equiv) was added. The reaction mixture was stirred at room temperature for 1.5 h and then the mixture was poured into sat. NH₄Cl (300 mL) and stirred for 5 min. The aqueous phase was extracted with EtOAc (3×80 mL) and the combined organic phases were washed with brine (50 mL), dried over anhydrous Na₂SO₄, filtered and concentrated under reduced pressure. The residue was purified by silica gel chromatography (20:1 to 1:1 petroleum ether/EtOAc) to afford tert-butyl N-tert-butoxycarbonyl-N-[(2-chloro pyrimidin-5-yl)methyl]carbamate (7.0 g, 60.3% yield) as a white solid. LCMS (ESI) m/z: [M+H] calcd for C₁₅H₂₂ClN₃O₄: 344.14; found 344.2.

### Step 2: Synthesis of tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate

To a solution of 8-(6-methoxy-3-pyridyl)-3-methyl-1-[4-piperazin-1-yl-3-(trifluoromethyl)phenyl]imidazo[4,5-c]quinolin-2-one (0.4 g, 748.32 μmol, 1.0 equiv) in MeCN (7 mL) was added tert-butyl N-tert-butoxycarbonyl-N-[(2-chloropyrimidin-5-yl)methyl]carbamate (514.55 mg, 1.50 mmol, 2.0 equiv) and K₂CO₃ (413.69 mg, 2.99 mmol, 4 equiv) at room temperature. The reaction mixture was stirred at 80° C. for 14 h and then the mixture was cooled to room temperature, filtered and concentrated under reduced pressure. The residue was purified by washing with MTBE (5 mL) to give tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate (0.57 g, 90.5% yield) as a light yellow solid. LCMS (ESI) m/z: [M+H] calcd for C₄₃H₄₆F₃N₉O₆: 842.36; found 842.7.

### Step 3: Synthesis of 1-[4-[4-[5-(aminomethyl)pyrimidin-2-yl]piperazin-1-yl]-3-(trifluoromethyl) phenyl]-8-(6-methoxy-3-pyridyl)-3-methyl-imidazo[4,5-c]quinolin-2-one

A solution of tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate (0.95 g, 1.13 mmol, 1 equiv) in TFA (10 mL) was stirred at room temperature for 1 h, at which point the solvent was concentrated. The residue was dissolved in MeCN (10 mL) and then the solution was added to MTBE (150 mL), dropwise. The precipitate was collected to give 1-[4-[4-[5-(aminomethyl)pyrimidin-2-yl]piperazin-1-yl]-3-(trifluoromethyl)phenyl]-8-(6-methoxy-3-pyridyl)-3-methyl-imidazo[4,5-c]quinolin-2-one trifluoromethanesulfonate (0.778 g, 84.8% yield) as a yellow solid. LCMS (ESI) m/z: [M+H] calcd for C₃₃H₃₀F₃N₉O₂: 642.26; found 642.4.

### Monomer W. 1-(4-aminobutyl)-3-(1H-pyrrolo[2,3-b]pyridin-5-yl)pyrazolo[3,4-d]pyrimidin-4-amine

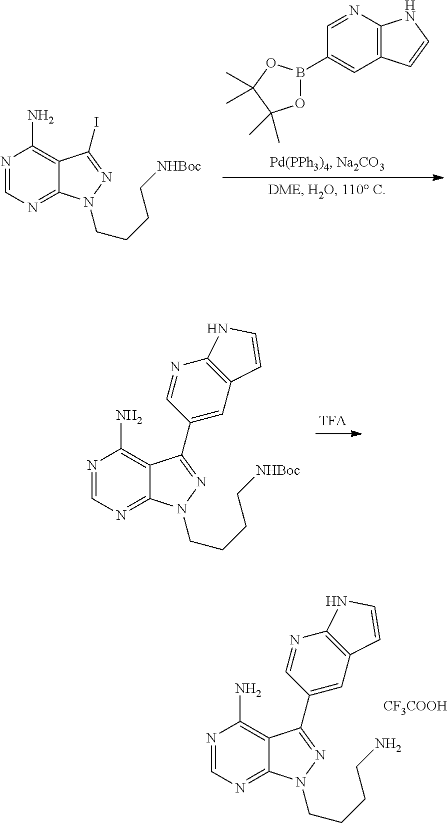

### Step 1: Synthesis of tert-butyl N-[4-[4-amino-3-(1H-indol-5-yl)pyrazolo[3,4-d]pyrimidin-1-yl]butyl]carbamate

To a bi-phasic suspension of tert-butyl N-[4-(4-amino-3-iodo-pyrazolo[3,4-d]pyrimidin-1-yl)butyl]carbamate (8 g, 18.51 mmol, 1 equiv), 5-(4,4,5,5-tetramethyl-1,3,2-dioxaborolan-2-yl)-1H-pyrrolo[2,3-b]pyridine (5.42 g, 22.21 mmol, 1.2 equiv) and Na₂CO₃ (9.81 g, 92.54 mmol, 5 equiv) in diglyme (160 mL) and H₂O (80 mL) was added Pd(PPh₃)₄ (2.14 g, 1.85 mmol, 0.1 equiv) at room temperature under N₂. The mixture was stirred at 110° C. for 3 h. The reaction mixture was cooled to room temperature, filtered and the filtrate was partitioned between EtOAc (500 mL) and H₂O (500 mL). The aqueous layer was separated and extracted with EtOAc (3×300 mL). The organic layers were combined, washed with brine (20 mL) and dried over anhydrous Na₂SO₄, then filtered and the filtrate was concentrated under reduced pressure. The residue was purified by silica gel chromatography (1/0 to 0/1 petroleum ether/EtOAc then 4/1 EtOAc/MeOH) to give tert-butyl N-[4-[4-amino-3-(1H-indol-5-yl)pyrazolo[3,4-d]pyrimidin-1-yl]butyl]carbamate (6.6 g, 84.6% yield) as a yellow solid. LCMS (ESI) m/z: [M+H] calcd for C₂₂H₂₇N₇O₂: 422.22; found 423.3.

### Step 2: Synthesis of 1-(4-aminobutyl)-3-(1H-pyrrolo[2,3-b]pyridin-5-yl)pyrazolo[3,4-d]pyrimidin-4-amine

To tert-butyl N-[4-[4-amino-3-(1H-indol-5-yl)pyrazolo[3,4-d]pyrimidin-1-yl]butyl]carbamate (6.6 g, 15.66 mmol, 1 equiv) was added TFA (66 mL), which was then stirred at room temperature for 30 min. The reaction solution was concentrated under reduced pressure to remove TFA and then MTBE (400 mL) was added to the residue. The suspension was stirred for 15 min, at which point the yellow solid was filtered, and the solid cake dried under reduced pressure to give 1-(4-aminobutyl)-3-(1H-pyrrolo[2,3-b]pyridin-5-yl)pyrazolo[3,4-d]pyrimidin-4-amine (10.2 g, 97.1% yield) as a yellow solid. LCMS (ESI) m/z: [M+H] calcd for C₆H₁₈N₈: 323.17; found 323.1.

### Monomer X. 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate

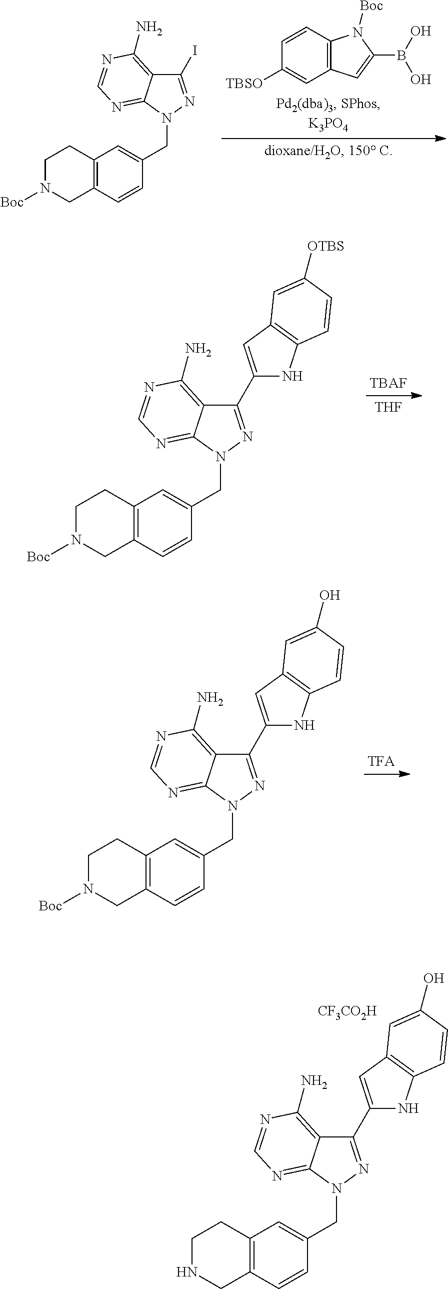

### Step 1: Synthesis of tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate

To a solution of tert-butyl 6-((4-amino-3-iodo-1H-pyrazolo[3,4-d]pyrimidin-1-yl)methyl)-3,4-dihydroisoquinoline-2(1H)-carboxylate (1 g, 1.97 mmol, 1.0 equiv) in dioxane (10.5 mL) and H₂O (3.5 mL) was added (1-(tert-butoxycarbonyl)-5-((tert-butyldimethylsilyl)oxy)-1H-indol-2-yl)boronic acid (1.16 g, 2.96 mmol, 1.5 equiv), K₃PO₄ (1.26 g, 5.92 mmol, 3.0 equiv), Pd₂(dba)₃ (180.85 mg, 197.50 μmol, 0.1 equiv), and SPhos (162.16 mg, 394.99 μmol, 0.2 equiv) at room temperature under N₂. The sealed tube was heated at 150° C. for 20 min under microwave. The reaction mixture was then cooled and 6 separate batches were combined together. The reaction mixture was partitioned between EtOAc (100 mL) and H₂O (100 mL). The aqueous layer was separated and extracted with EtOAc (3×80 mL). The organic layers were combined, washed with brine (100 mL) and dried over anhydrous Na₂SO₄. The solution was filtered and the filtrate was concentrated under reduced pressure. The crude material was purified by silica gel column chromatography (100/1 to 1/4 petroleum ether/EtOAc) to give tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate (6.17 g, 82.9% yield) as a light yellow solid.

### Step 2: Synthesis of tert-butyl 6-((4-amino-3-(5-hydroxy-1H-indol-2-yl)-1H-pyrazolo[3,4-d]pyrimidin-1-yl)methyl)-3,4-dihydroisoquinoline-2(1H)-carboxylate

To a mixture of tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate (6.17 g, 9.86 mmol, 1.0 equiv) in THF (100 mL) was added tetrabutylammonium fluoride trihydrate (1 M, 10.84 mL, 1.1 equiv) in one portion at 0° C. under N₂. The mixture was stirred at 0° C. for 1 h and was then added to H₂O (100 mL). The aqueous phase was extracted with EtOAc (3×80 mL) and the combined organic phase was washed with brine (2×80 mL), dried with anhydrous Na₂SO₄, filtered and concentrated under reduced pressure. The residue was purified by silica gel chromatography (1/1 to 0/1 petroleum ether/EtOAc) to afford tert-butyl 6-((4-amino-3-(5-hydroxy-1H-indol-2-yl)-1H-pyrazolo[3,4-d]pyrimidin-1-yl)methyl)-3,4-dihydroisoquinoline-2(1H)-carboxylate (4 g, 79.3% yield) as a light pink solid. LCMS (ESI) m/z: [M+H] calcd for C₂₈H₂₉N₇O₃: 512.24; found 512.3.

### Step 3: Synthesis of 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate

To a solution of tert-butyl 6-((4-amino-3-(5-hydroxy-1H-indol-2-yl)-1H-pyrazolo [3,4-d]pyrimidin-1-yl)methyl)-3,4-dihydroisoquinoline-2(1H)-carboxylate (4.5 g, 8.80 mmol, 1.0 equiv) in MeOH (50 mL) was added HCl in MeOH (4 M, 50 mL, 22.7 equiv) at room temperature. The mixture was stirred at room temperature overnight and was then concentrated under reduced pressure. To the crude product was added EtOAc (100 mL) and the resulting precipitate was collected by filtration under N₂ to give 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate (4.1 g, 85.0% yield, 3HCl) as a light yellow solid. LCMS (ESI) m/z: [M+H] calcd for C₂₃H₂₁N₇O: 412.19; found 412.1.

### Monomer Y. 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate

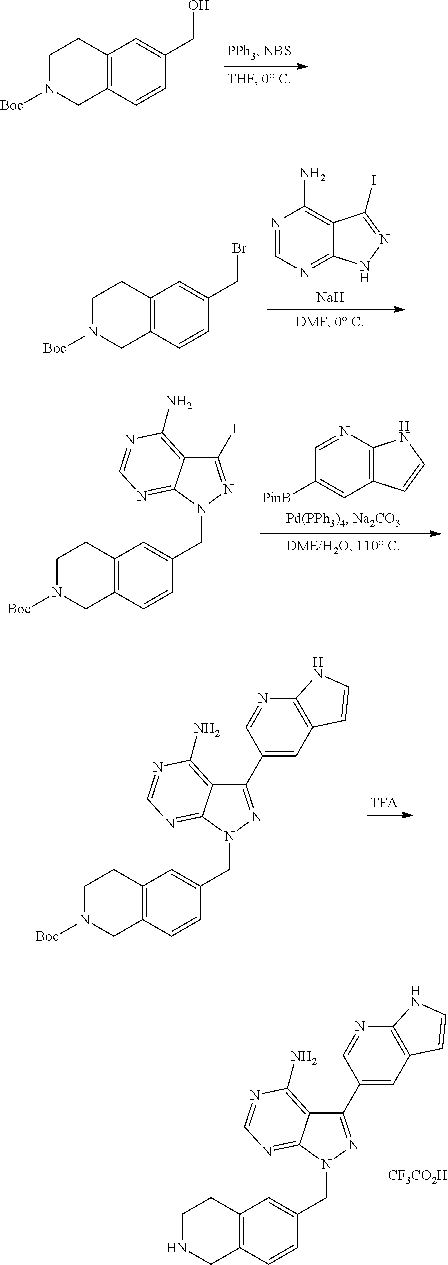

### Step 1: Synthesis of tert-butyl 6-(bromomethyl)-3,4-dihydroisoquinoline-2(1H)-carboxylate

A solution of NBS (34.07 g, 191.39 mmol, 4 equiv) in THF (200 mL) was added in portions to a solution of tert-butyl 6-(hydroxymethyl)-3,4-dihydroisoquinoline-2(1H)-carboxylate (12.6 g, 47.85 mmol, 1.0 equiv) and triphenylphosphine (37.65 g, 143.55 mmol, 3.0 equiv) in THF (200 mL) at 0° C. After the addition was complete, the mixture was stirred for 1 h at room temperature. EtOAc (150 mL) was added and the mixture was washed with H₂O (200 mL) and brine (150 mL), dried over anhydrous Na₂SO₄ and concentrated under reduced pressure. The residue was purified by silica gel chromatography (100/i to 10/1 petroleum ether/EtOAc) to afford tert-butyl 6-(bromomethyl)-3,4-dihydroisoquinoline-2(1H)-carboxylate (8.56 g, 54.8% yield) as a light yellow solid.

### Step 2: Synthesis of tert-butyl 6-((4-amino-3-iodo-1H-pyrazolo[3,4-d]pyrimidin-1-yl) methyl)-3,4-dihydroisoquinoline-2(1H)-carboxylate

To a suspension of 3-iodo-1H-pyrazolo[3,4-d]pyrimidin-4-amine (9.5 g, 36.40 mmol, 1.0 equiv) in DMF (110 mL) was added NaH (1.46 g, 36.40 mmol, 60 wt. %, 1.0 equiv) at 0° C. The mixture was stirred at 0° C. for 30 min at which point a solution of tert-butyl 6-(bromomethyl)-3,4-dihydroisoquinoline-2(1H)-carboxylate (12.47 g, 38.22 mmol, 1.05 equiv) in DMF (40 mL) was added at 0° C. The mixture was stirred at room temperature for 1 h and then H₂O (1000 mL) was added at 0° C. The mixture stirred at 0° C. for 30 min and then the resulting precipitate was collected by filtration to give tert-butyl 6-((4-amino-3-iodo-1H-pyrazolo[3,4-d]pyrimidin-1-yl)methyl)-3,4-dihydroisoquinoline-2(1H)-carboxylate (17.8 g, 76.3% yield) as a light yellow solid, which was used the next step directly. LCMS (ESI) m/z: [M+H] calcd for C₂₀H₂₃IN₆O₂: 507.10; found 507.1.

### Step 3: Synthesis of tert-butyl 6-((4-amino-3-(1H-pyrrolo[2,3-b]pyridin-5-yl)-1H-pyrazolo[3,4-d]pyrimidin-1-yl)methyl)-3,4-dihydroisoquinoline-2(1H)-carboxylate

To a bi-phasic suspension of tert-butyl 6-((4-amino-3-iodo-1H-pyrazolo[3,4-d]pyrimidin-1-yl)methyl)-3,4-dihydroisoquinoline-2(1H)-carboxylate (6.5 g, 10.14 mmol, 1.0 equiv), 5-(4,4,5,5-tetramethyl-1,3,2-dioxaborolan-2-yl)-1H-pyrrolo[2,3-b]pyridine (2.97 g, 12.16 mmol, 1.2 equiv), and Na₂CO₃ (5.37 g, 50.68 mmol, 5.0 equiv) in diglyme (100 mL) and H₂O (50 mL) was added Pd(PPh₃)₄ (1.17 g, 1.01 mmol, 0.1 equiv) at room temperature under N₂. The mixture was stirred at 110° C. for 3 h. The reaction mixture was then cooled and partitioned between EtOAc (100 mL) and H₂O (100 mL). The aqueous layer was separated and extracted with EtOAc (2×100 mL). The combined organic phase was washed with brine (100 mL), dried with anhydrous Na₂SO₄, filtered and concentrated under reduced pressure. The residue was purified by silica gel chromatography (0/1 to 1/4 MeOH/EtOAc) to afford tert-butyl 6-((4-amino-3-(1H-pyrrolo[2,3-b]pyridin-5-yl)-1H-pyrazolo[3,4-d]pyramidin-1-yl) methyl)-3,4-dihydroisoquinoline-2(1H)-carboxylate (3.77 g, 72.1% yield) as a light yellow solid. LCMS (ESI) m/z: [M+H] calcd for C₂₇H₂₈N₈O₂: 497.24; found 497.3.

### Step 4: Synthesis of 3-(1H-pyrrolo[2,3-b]pyridin-5-yl)-1-((1,2,3,4-tetrahydroiso quinolin-6-yl)methyl)-1H-pyrazolo[3,4-d]pyrimidin-4-amine 2,2,2-trifluoroacetate

tert-Butyl 6-((4-amino-3-(1H-pyrrolo[2,3-b]pyridin-5-yl)-1H-pyrazolo[3,4-d]pyrimidin-1-yl)methyl)-3,4-dihydroisoquinoline-2(1H)-carboxylate (3.77 g, 7.59 mmol, 1.0 equiv) was added to TFA (85.36 mL, 1.15 mol, 151.8 equiv) at room temperature. The reaction mixture was stirred for 1 h. It was then concentrated under reduced pressure and the oily residue was triturated with MeCN (3 mL), then dripped into MTBE (200 mL) for 5 min. The supernatant was removed and then the precipitate was collected by filtration under N₂ to give the product, which was dissolved in MeCN (20 mL), and finally concentrated under reduced pressure to give 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate (4.84 g, 85.0% yield, 3TFA) as a light yellow solid. LCMS (ESI) m/z: [M+H] calcd for C₂₂H₂₀N₈: 397.19; found 397.2.

### Monomer Z. (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate

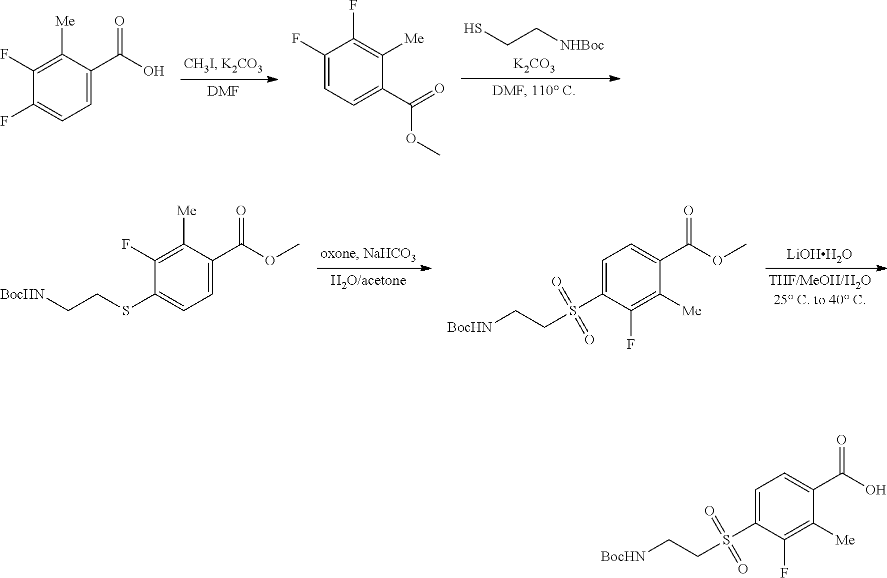

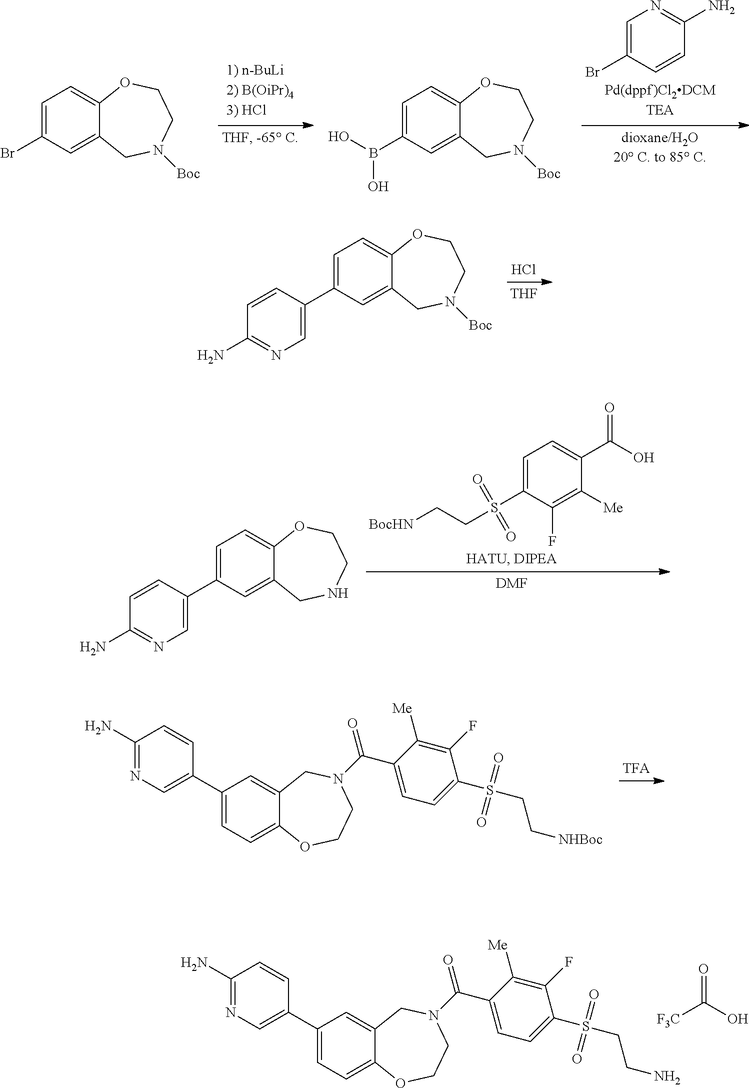

### Step 1: Synthesis of methyl 3,4-difluoro-2-methylbenzoate

To a solution of 3,4-difluoro-2-methylbenzoic acid (2 g, 11.62 mmol, 1.0 equiv) in DMF (20 mL) was added K₂CO₃ (4.82 g, 34.86 mmol, 3.0 equiv) and iodomethane (3.26 mL, 52.29 mmol, 4.5 equiv) at room temperature. The mixture was stirred at room temperature for 3 h. The solution of methyl 3,4-difluoro-2-methylbenzoate in DMF (20 mL) was used directly in the next step.

### Step 2: Synthesis of methyl 4-((2-((tert-butoxycarbonyl)amino)ethyl)thio)-3-fluoro-2-methylbenzoate

To a solution of methyl 3,4-difluoro-2-methylbenzoate (2.16 g, 11.28 mmol, 1.0 equiv) in DMF (20 mL) was added tert-butyl (2-mercaptoethyl)carbamate (2.0 g, 11.28 mmol, 1 equiv) and K₂CO₃ (3.12 g, 22.56 mmol, 2.0 equiv) at room temperature. The reaction was stirred at 110° C. for 12 h, at which point the mixture was added to H₂O (50 mL). The aqueous solution was then extracted with EtOAc (3×30 mL) and the organic phase was combined and concentrated under reduced pressure. The residue was purified by silica gel chromatography (1/0 to 3/1 petroleum ether/EtOAc) to afford methyl 4-((2-((tert-butoxycarbonyl)amino)ethyl)thio)-3-fluoro-2-methylbenzoate (3.0 g, 76% yield) as light yellow solid.

### Step 3: Synthesis of methyl 4-((2-((tert-butoxycarbonyl)amino)ethyl)sulfonyl)-3-fluoro-2-methylbenzoate

To a solution of methyl 4-((2-((tert-butoxycarbonyl)amino)ethyl)thio)-3-fluoro-2-methylbenzoate (3.3 g, 9.61 mmol, 1.0 equiv), NaOH (2 M, 4.80 mL, 1.0 equiv), and NaHCO₃ (2.42 g, 28.83 mmol, 3.0 equiv) in acetone (30 mL) was added potassium peroxymonosulfate (12.35 g, 20.08 mmol, 2.1 equiv). The mixture was stirred for 12 h at room temperature and then the mixture was acidified to pH 5 by addition of 1N HCl. The aqueous layer was extracted with EtOAc (3×30 mL) and the combined organic phase was washed with brine (20 mL), dried with anhydrous Na₂SO₄, filtered and concentrated under reduced pressure. The residue was purified by silica gel chromatography (1/0 to 3/1 petroleum ether/EtOAc) to afford methyl 4-((2-((tert-butoxycarbonyl)amino)ethyl)sulfonyl)-3-fluoro-2-methylbenzoate (2.1 g, 58.2% yield) as a yellow solid. LCMS (ESI) m/z: [M−56+H] calcd for C₁₆H₂₂FNO₆S: 320.12; found 320.1.

### Step 4: Synthesis of 4-((2-((tert-butoxycarbonyl)amino)ethyl)sulfonyl)-3-fluoro-2-methylbenzoic acid

To a solution of methyl 4-((2-((tert-butoxycarbonyl)amino)ethyl)sulfonyl)-3-fluoro-2-methylbenzoate (2.1 g, 5.59 mmol, 1.0 equiv) in THF (20 mL), MeOH (10 mL) and H₂O (10 mL) was added LiOH·H₂O (704.16 mg, 16.78 mmol, 3.0 equiv) at room temperature. The reaction mixture was stirred at 40° C. for 4 h. The mixture was then concentrated under reduced pressure to remove THF and MeOH. The aqueous phase was neutralized with 0.5N HCl and was then extracted with EtOAc (5×20 mL). The combined organic phase was washed with brine (2×20 mL), dried with anhydrous Na₂SO₄, filtered and concentrated under reduced pressure to give 4-((2-((tert-butoxycarbonyl)amino)ethyl)sulfonyl)-3-fluoro-2-methylbenzoic acid (2.01 g, 97.1% yield) as a white solid. LCMS (ESI) m/z: [M−100+H] calcd for C₁₅H₂₀FNO₆S: 262.11; found 262.1.

### Step 5: Synthesis of (4-(tert-butoxycarbonyl)-2,3,4,5-tetrahydrobenzo[f][1,4] oxazepin-7-yl)boronic acid

To a solution of tert-butyl 7-bromo-2,3-dihydrobenzo[f][1,4]oxazepine-4(5H)-carboxylate (4 g, 12.19 mmol, 1.0 equiv) in THF (80 mL) at −60° C. was added B(OiPr)₃ (4.58 g, 24.38 mmol, 5.60 mL, 2.0 equiv) followed by dropwise addition of n-BuLi (2.5 M, 12.19 mL, 2.5 equiv) in n-hexane. The reaction was stirred at −65° C. for 1 h. The reaction mixture was quenched with 1N HCl (12.25 mL) and allowed to warm to room temperature. The reaction mixture was extracted with EtOAc (3×30 mL), dried over anhydrous Na₂SO₄, filtered and concentrated under reduced pressure to give (4-(tert-butoxycarbonyl)-2,3,4,5-tetrahydrobenzo[f][1,4]oxazepin-7-yl)boronic acid (3.5 g, crude) as light yellow oil, which was used to the next step directly. LCMS (ESI) m/z: [M−100+H] calcd for C₁₄H₂₀BNO₅: 194.15; found 194.2.

### Step 6: Synthesis of tert-butyl 7-(6-aminopyridin-3-yl)-2,3-dihydrobenzo[f][1,4] oxazepine-4(5H)-carboxylate

To a solution of (4-(tert-butoxycarbonyl)-2,3,4,5-tetrahydrobenzo[f][1,4]oxazepin-7-yl)boronic acid (4.2 g, 14.33 mmol, 1.0 equiv) in H₂O (20 mL) and dioxane (60 mL) was added 5-bromopyridin-2-amine (2.48 g, 14.33 mmol, 1.0 equiv), Pd(dppf)Cl₂·DCM (1.17 g, 1.43 mmol, 0.1 equiv) and Et₃N (4.35 g, 42.99 mmol, 5.98 mL, 3.0 equiv) at room temperature. The mixture was stirred at 85° C. for 12 h. The mixture was then cooled to room temperature and the residue was poured into H₂O (15 mL). The aqueous phase was extracted with EtOAc (3×40 mL) and the combined organic phase was washed with brine (2×40 mL), dried with anhydrous Na₂SO₄, filtered and concentrated under reduced pressure. The residue was purified by silica gel chromatography (1/0 to 1/8 petroleum ether/EtOAc) to afford tert-butyl 7-(6-aminopyridin-3-yl)-2,3-dihydrobenzo[f][1,4]oxazepine-4(5H)-carboxylate (3.3 g, 65.0% yield) as light yellow solid. LCMS (ESI) m/z: [M+H] calcd for C₁₉H₂₃N₃O₃: 342.18; found 342.2.

### Step 7: Synthesis of 5-(2,3,4,5-tetrahydrobenzo[f][1,4]oxazepin-7-yl)pyridin-2-amine

To a solution of tert-butyl 7-(6-aminopyridin-3-yl)-2,3-dihydrobenzo[f][1,4]oxazepine-4(5H)-carboxylate (3.3 g, 9.67 mmol, 1.0 equiv) in THF (40 mL) was added HCl in EtOAc (4 M, 100 mL, 41.38 equiv) at room temperature. The mixture was stirred for 3 h. The reaction mixture was filtered and the filter cake was washed with EtOAc (3×15 mL) and then dried under reduced pressure to give 5-(2,3,4,5-tetrahydrobenzo[f][1,4]oxazepin-7-yl)pyridin-2-amine (3 g, 95.1% yield, 2HCl) as a light yellow solid.

### Step 8: Synthesis of tert-butyl (2-((4-(7-(6-aminopyridin-3-yl)-2,3,4,5-tetrahydrobenzo[f][1,4]oxazepine-4-carbonyl)-2-fluoro-3-methylphenyl)sulfonyl)ethyl)carbamate

To a solution of 4-((2-((tert-butoxycarbonyl)amino)ethyl)sulfonyl)-3-fluoro-2-methylbenzoic acid (690.08 mg, 1.91 mmol, 1.0 equiv) in DMF (10 mL) was added HATU (1.09 g, 2.86 mmol, 1.5 equiv) and DIPEA (1.66 mL, 9.55 mmol, 5 equiv). The reaction was stirred at room temperature for 30 min and then 5-(2,3,4,5-tetrahydrobenzo[f][1,4]oxazepin-7-yl)pyridin-2-amine (0.6 g, 1.91 mmol, 1.0 equiv, 2HCl) was added. The mixture was stirred for 2 h, at which point H₂O (40 mL) was added. The mixture was stirred for 5 min and the resulting precipitate was collected by filtration to give the crude product. The residue was purified by silica gel chromatography (1/0 to 10/1 EtOAc/MeOH) to afford tert-butyl (2-((4-(7-(6-aminopyridin-3-yl)-2,3,4,5-tetrahydrobenzo[f][1,4] oxazepine-4-carbonyl)-2-fluoro-3-methylphenyl)sulfonyl)ethyl)carbamate (0.538 g, 47.4% yield) as a light yellow solid. LCMS (ESI) m/z: [M+H] calcd for C₂₉H₃₃FN₄O₆S: 585.22; found 585.3.

### Step 9: Synthesis of (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate

A solution tert-butyl (2-((4-(7-(6-aminopyridin-3-yl)-2,3,4,5-tetrahydrobenzo[f][1,4] oxazepine-4-carbonyl)-2-fluoro-3-methylphenyl)sulfonyl)ethyl)carbamate (0.538 g, 920.20 μmol, 1.0 equiv) in TFA (10.35 mL, 139.74 mmol, 151.85 equiv) was stirred at room temperature for 2 h. The solution was then concentrated under reduced pressure. The oily residue was triturated with MeCN (1 mL) and then dripped into MTBE (30 mL) for 10 min. The supernatant was removed and then the precipitate was collected by filtration under N₂ to give (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate (0.50 g, 87.4% yield) as light brown solid. LCMS (ESI) m/z: [M+H] calcd for C₂₄H₂₅FN₄O₄S: 485.17; found 485.1.

### Monomer AA. 5-(4-amino-1-(6-(piperazin-1-yl)pyrimidin-4-yl)-1H-pyrazolo[3,4-d]pyrimidin-3-yl)benzo[d]oxazol-2-amine trifluoroacetic acid salt

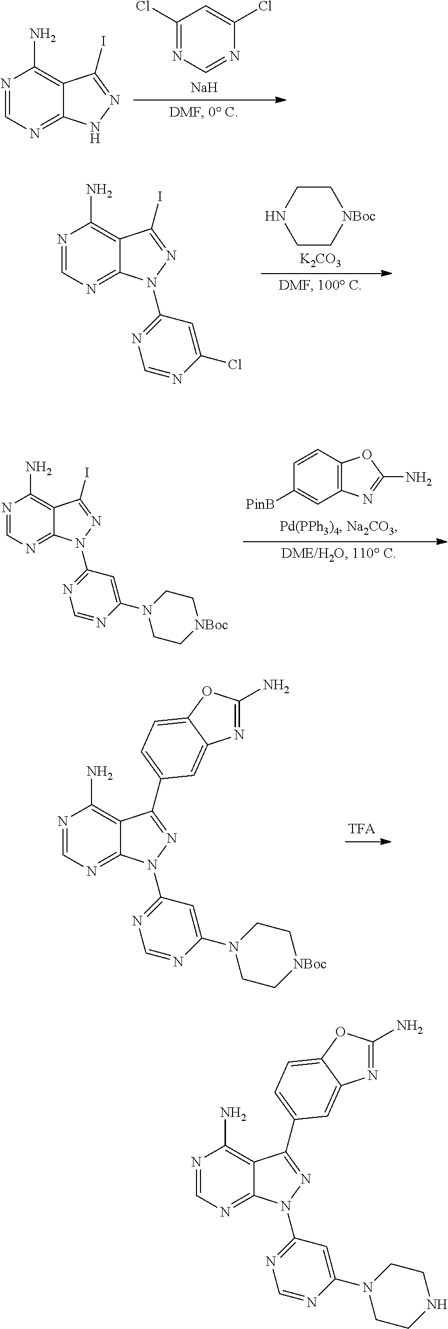

### Step 1: Synthesis of 1-(6-chloropyrimidin-4-yl)-3-iodo-1H-pyrazolo[3,4-d]pyrimidin-4-amine

To a suspension of 3-iodo-1H-pyrazolo[3,4-d]pyrimidin-4-amine (5 g, 19.16 mmol, 1.0 equiv) in DMF (60 mL) was added NaH (804.53 mg, 20.11 mmol, 60 wt. %, 1.05 equiv) at 0° C. The mixture was stirred at 0° C. for 30 min. To the reaction mixture was then added 4,6-dichloropyrimidine (3.42 g, 22.99 mmol, 1.2 equiv) at 0° C. The mixture was stirred at room temperature for 2.5 h, at which point the reaction mixture was added to H₂O (600 mL). The suspension was then filtered to give the product (7.1 g, 99.2% yield) as yellow solid. LCMS (ESI) m/z: [M+H] calcd for C₉H₅ClIN₇: 373.94; found 373.9.

### Step 2: Synthesis of tert-butyl 4-(6-(4-amino-3-iodo-1H-pyrazolo[3,4-d]pyrimidin-1-yl)pyrimidin-4-yl)piperazine-1-carboxylate

To a solution of 1-(6-chloropyrimidin-4-yl)-3-iodo-1H-pyrazolo[3,4-d]pyrimidin-4-amine (5 g, 13.39 mmol, 1.0 equiv) and tert-butyl piperazine-1-carboxylate (2.99 g, 16.06 mmol, 1.2 equiv) in DMF (50 mL) was added K₂CO₃ (3.70 g, 26.77 mmol, 2.0 equiv). The reaction mixture was stirred at 100° C. for 4 h, at which point it was added to H₂O (500 mL). The suspension was then filtered to give the product (6.2 g, 88.5% yield) as yellow solid. LCMS (ESI) m/z: [M+H] calcd for C₁₈H₂₂IN₉O₂: 524.09; found 524.2.

### Step 3: Synthesis of tert-butyl 4-(6-(4-amino-3-(2-aminobenzo[d]oxazol-5-yl)-1H-pyrazolo[3,4-d]pyrimidin-1-yl)pyrimidin-4-yl)piperazine-1-carboxylate

To a bi-phasic suspension of 5-(4,4,5,5-tetramethyl-1,3,2-dioxaborolan-2-yl)benzo[d]oxazol-2-amine (3.08 g, 11.85 mmol, 1.0 equiv), tert-butyl 4-(6-(4-amino-3-iodo-1H-pyrazolo[3,4-d]pyrimidin-1-yl)pyrimidin-4-yl)piperazine-1-carboxylate (6.2 g, 11.85 mmol, 1.0 equiv) and Na₂CO₃ (6.28 g, 59.24 mmol, 5.0 equiv) in H₂O (100 mL) and DME (200 mL) was added Pd(PPh₃)₄ (1.37 g, 1.18 mmol, 0.1 equiv) at room temperature under N₂. The mixture was stirred at 110° C. for 24 h and then the mixture was filtered to give a solid cake. The solid was added to dioxane (20 mL) and stirred at 110° C. for 60 min, then filtered to give the product (3.5 g, 55.8% yield) as brown solid. LCMS (ESI) m/z: [M+H] calcd for C₂₅H₂₇N₁₁O₃: 530.24; found 530.3.

### Step 4: Synthesis of 5-(4-amino-1-(6-(piperazin-1-yl)pyrimidin-4-yl)-1H-pyrazolo[3,4-d]pyrimidin-3-yl)benzo[d]oxazol-2-amine trifluoroacetic acid salt

A solution of tert-butyl 4-(6-(4-amino-3-(2-aminobenzo[d]oxazol-5-yl)-1H-pyrazolo[3,4-d]pyrimidin-1-yl)pyrimidin-4-yl)piperazine-1-carboxylate (3.5 g, 6.61 mmol, 1.0 equiv) in TFA (35 mL) was stirred at room temperature for 1 h. The reaction solution was concentrated under reduced pressure and the resulting crude material was dissolved in MeCN (20 mL) and added dropwise to MTBE (500 mL). The resulting solid was then filtered to give the product (5.5 g, 91.9% yield) as brown solid. LCMS (ESI) m/z: [M+H] calcd for C₂₀H₁₉N₁₁O: 430.19; found 430.1.

### Monomer AB. 8-(6-methoxypyridin-3-yl)-3-methyl-1-(4-(4-(5,6,7,8-tetrahydropyrido[4,3-d]pyrimidin-2-yl)piperazin-1-yl)-3-(trifluoromethyl)phenyl)-1H-imidazo[4,5-c]quinolin-2(3H)-one trifluoroacetic acid salt

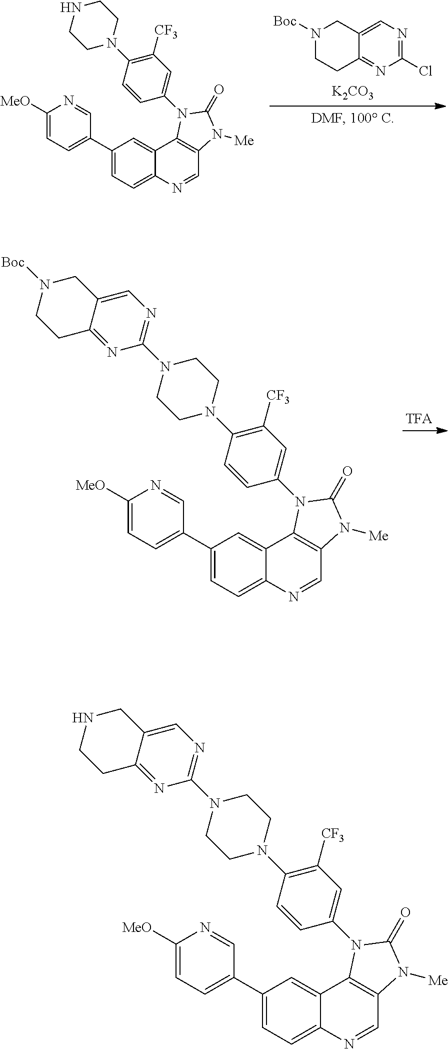

### Step 1: Synthesis of tert-butyl 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)-7,8-dihydropyrido[4,3-d]pyrimidine-6(5H)-carboxylate

To a mixture of 8-(6-methoxypyridin-3-yl)-3-methyl-1-(4-(piperazin-1-yl)-3-(trifluoromethyl)phenyl)-1H-imidazo[4,5-c]quinolin-2(3H)-one (0.3 g, 561.24 μmol, 1.0 equiv) and tert-butyl 2-chloro-7,8-dihydropyrido[4,3-d]pyrimidine-6(5H)-carboxylate (151.38 mg, 561.24 μmol, 1.0 equiv) in DMF (5 mL) was added K₂CO₃ (193.92 mg, 1.40 mmol, 2.5 equiv). The mixture was stirred at 100° C. for 14 h, at which point H₂O (20 mL) was added. The aqueous layer was extracted with EtOAc (3×40 mL) and the combined organic layers were concentrated under reduced pressure. The crude material was purified by column chromatography (30/1 to 15/1 DCM/MeOH) to give the product (0.30 g, 69.6% yield) as a light-yellow solid. LCMS (ESI) m/z: [M+H] calcd for C₄₀H₄₀F₃N₉O₄: 768.33; found 768.5.

### Step 2: Synthesis of 8-(6-methoxypyridin-3-yl)-3-methyl-1-(4-(4-(5,6,7,8-tetrahydropyrido[4,3-d]pyrimidin-2-yl)piperazin-1-yl)-3-(trifluoromethyl)phenyl)-1H-imidazo[4,5-c]quinolin-2(3H)-one

A solution of tert-butyl 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)-7,8-dihydropyrido[4,3-d]pyrimidine-6(5H)-carboxylate (0.8 g, 1.04 mmol, 1.0 equiv) in TFA (8 mL) was stirred at room temperature for 2 h. The solvent was concentrated and the residue was dissolved in MeCN (5 mL), then the solution was added dropwise to MTBE (150 mL). The precipitate was filtered and the solid was dried under reduced pressure to give the product (600 mg, 70.6% yield) as a yellow solid. LCMS (ESI) m/z: [M+H] calcd for C₃₅H₃₂F₃N₉O₂: 668.27; found 668.3.

### Monomer AC. 5-(4-amino-1-(piperidin-4-ylmethyl)-1H-pyrazolo[3,4-d]pyrimidin-3-yl)benzo[d]oxazol-2-amine trifluoroacetic acid salt

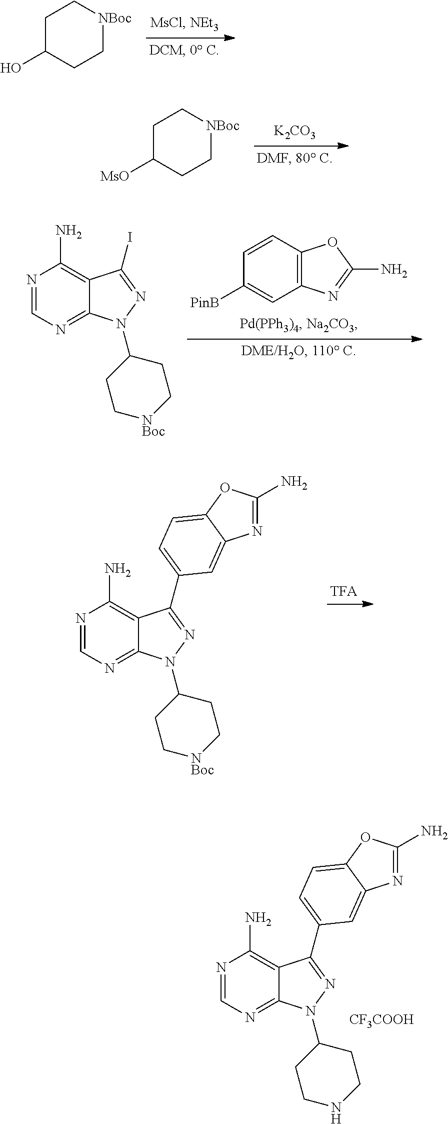

### Step 1: Synthesis of tert-butyl 4-((methylsulfonyl)oxy)piperidine-1-carboxylate

To a solution of tert-butyl 4-hydroxypiperidine-1-carboxylate (4 g, 19.87 mmol, 1.0 equiv) and Et₃N (3.87 mL, 27.82 mmol, 1.4 equiv) in DCM (40 mL) was added MsCl (2.15 mL, 27.82 mmol, 1.4 equiv) at 0° C. Then the reaction mixture was stirred at room temperature for 1 h. H₂O (50 mL) was added and the aqueous phase was extracted with DCM (3×50 mL). The combined organic phase was washed with brine, dried with anhydrous Na₂SO₄, filtered and concentrated under reduced pressure to give the product (5.62 g, 101% crude yield) as yellow solid which was used directly in the next step.

### Step 2: Synthesis of tert-butyl 4-(4-amino-3-iodo-1H-pyrazolo[3,4-d]pyrimidin-1-yl)piperidine-1-carboxylate

To a suspension of 3-iodo-1H-pyrazolo[3,4-d]pyrimidin-4-amine (5 g, 19.16 mmol, 1.0 equiv) and tert-butyl 4-((methylsulfonyl)oxy)piperidine-1-carboxylate (5.62 g, 20.11 mmol, 1.05 equiv) in DMF (100 mL) was added K₂CO₃ (5.29 g, 38.31 mmol, 2.0 equiv). The mixture was stirred at 80° C. for 12 h. The reaction mixture was then added to H₂O (400 mL) at 0° C. The resulting precipitate was filtered to give the product (5.0 g, 58.8% yield) as yellow solid. LCMS (ESI) m/z: [M+H] calcd for C₁₅H₂₁IN₆O₂: 445.09; found 445.1.

### Step 3: Synthesis of tert-butyl 4-(4-amino-3-(2-aminobenzo[d]oxazol-5-yl)-1H-pyrazolo[3,4-d]pyrimidin-1-yl)piperidine-1-carboxylate

To a suspension of tert-butyl 4-(4-amino-3-iodo-1H-pyrazolo[3,4-d]pyrimidin-1-yl)piperidine-1-carboxylate (5 g, 11.25 mmol, 1.0 equiv), 5-(4,4,5,5-tetramethyl-1,3,2-dioxaborolan-2-yl)benzo[d]oxazol-2-amine (3.51 g, 13.51 mmol, 1.2 equiv) and Na₂CO₃ (5.96 g, 56.27 mmol, 5.0 equiv) in H₂O (50 mL) and DME (100 mL) was added Pd(PPh₃)₄ (1.30 g, 1.13 mmol, 0.1 equiv) at room temperature under N₂. The mixture was stirred at 110° C. for 3 h. The reaction mixture was then cooled to room temperature and filtered. The filtrate was partitioned between EtOAc (100 mL) and H₂O (100 mL) and then the aqueous layer was separated and extracted with EtOAc (3×100 mL). The combined organic layer was washed with brine (20 mL) and dried over anhydrous Na₂SO₄, filtered, and concentrated under reduced pressure. The residue was triturated with EtOAc (30 mL) and filtered to give the product (3.6 g, 71% yield) as yellow solid. LCMS (ESI) m/z: [M+H] calcd for C₂₂H₂₆N₈O₃: 451.22; found 451.3.

### Step 4: Synthesis of 5-(4-amino-1-(piperidin-4-yl)-1H-pyrazolo[3,4-d]pyrimidin-3-yl)benzo[d]oxazol-2-amine trifluoroacetic acid salt

A solution of tert-butyl 4-(4-amino-3-(2-aminobenzo[d]oxazol-5-yl)-1H-pyrazolo[3,4-d]pyrimidin-1-yl)piperidine-1-carboxylate (1.4 g, 3.11 mmol, 1.0 equiv) in TFA (10 mL) was stirred at room temperature for 30 min. The reaction solution was concentrated under reduced pressure and the crude solid was dissolved in MeCN (20 mL). The solution was added dropwise to MTBE (100 mL) and the resulting solid was filtered to give the product (1.6 g, 85.8% yield) as yellow solid. LCMS (ESI) m/z: [M+H] calcd for C₁₇H₁₈N₈O₃: 351.17; found 351.1.

### Monomer AD. 1-(piperidin-4-yl)-3-(1H-pyrrolo[2,3-b]pyridin-5-yl)-1H-pyrazolo[3,4-d]pyrimidin-4-amine trifluoroacetic acid salt

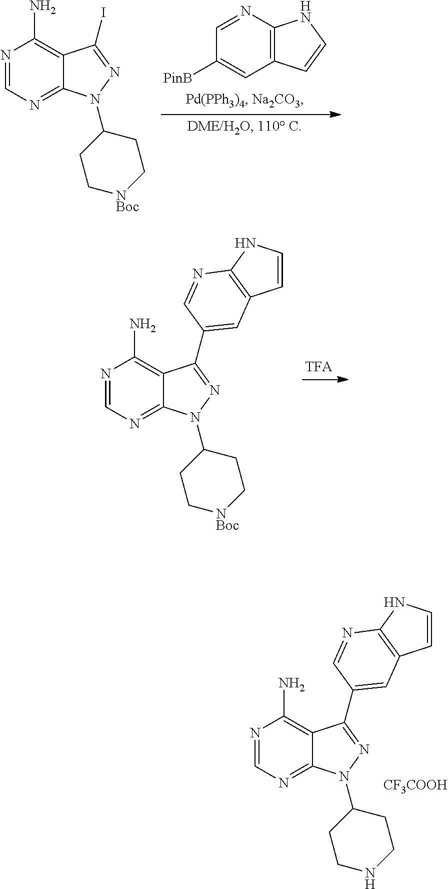

### Step 1: Synthesis of tert-butyl 4-(4-amino-3-(1H-pyrrolo[2,3-b]pyridin-5-yl)-1H-pyrazolo[3,4-d]pyrimidin-1-yl)piperidine-1-carboxylate

To a suspension of 5-(4,4,5-trimethyl-1,3,2-dioxaborolan-2-yl)-1H-pyrrolo[2,3-b]pyridine (857.12 mg, 3.51 mmol, 1.2 equiv), tert-butyl 4-(4-amino-3-iodo-1H-pyrazolo[3,4-d]pyrimidin-1-yl)piperidine-1-carboxylate (1.3 g, 2.93 mmol, 1.0 equiv) and Na₂CO₃ (1.55 g, 14.63 mmol, 5.0 equiv) in DME (20 mL) and H₂O (10 mL) was added Pd(PPh₃)₄ (338.13 mg, 292.62 μmol, 0.1 equiv) at room temperature under N₂. The mixture was stirred at 110° C. for 3 h. The reaction mixture was then cooled to room temperature and filtered. The filtrate was partitioned between EtOAc (50 mL) and H₂O (50 mL) and the aqueous layer was separated and extracted with EtOAc (3×50 mL). The combined organic layer were washed with brine, dried over anhydrous Na₂SO₄, filtered, and concentrated under reduced pressure. The residue was triturated with EtOAc (10 mL), filtered, the solid cake was dried under reduced pressure to give the product (1.0 g, 78.7% yield) as yellow solid.

### Step 2: Synthesis of 1-(piperidin-4-yl)-3-(1H-pyrrolo[2,3-b]pyridin-5-yl)-1H-pyrazolo[3,4-d]pyrimidin-4-amine trifluoroacetic acid salt

A solution of tert-butyl 4-(4-amino-3-(1H-pyrrolo[2,3-b]pyridin-5-yl)-1H-pyrazolo[3,4-d]pyrimidin-1-yl)piperidine-1-carboxylate (1.5 g, 3.45 mmol, 1.0 equiv) in TFA (10 mL) was stirred at room temperature for 30 min. The reaction solution was concentrated under reduced pressure and the crude residue was dissolved in MeCN (20 mL). The solution was added dropwise to MTBE (100 mL) and the resulting solid was filtered to give the product (1.19 g, 74.2% yield) as light yellow solid. LCMS (ESI) m/z: [M+H] calcd for C₁₇H₁₈N₈: 335.18; found 335.1.

### Monomer AE. (4-((2-aminoethyl)sulfonyl)-2-methylphenyl)(7-(6-aminopyridin-3-yl)-2,3-dihydrobenzo[f][1,4]oxazepin-4(5H)-yl)methanone

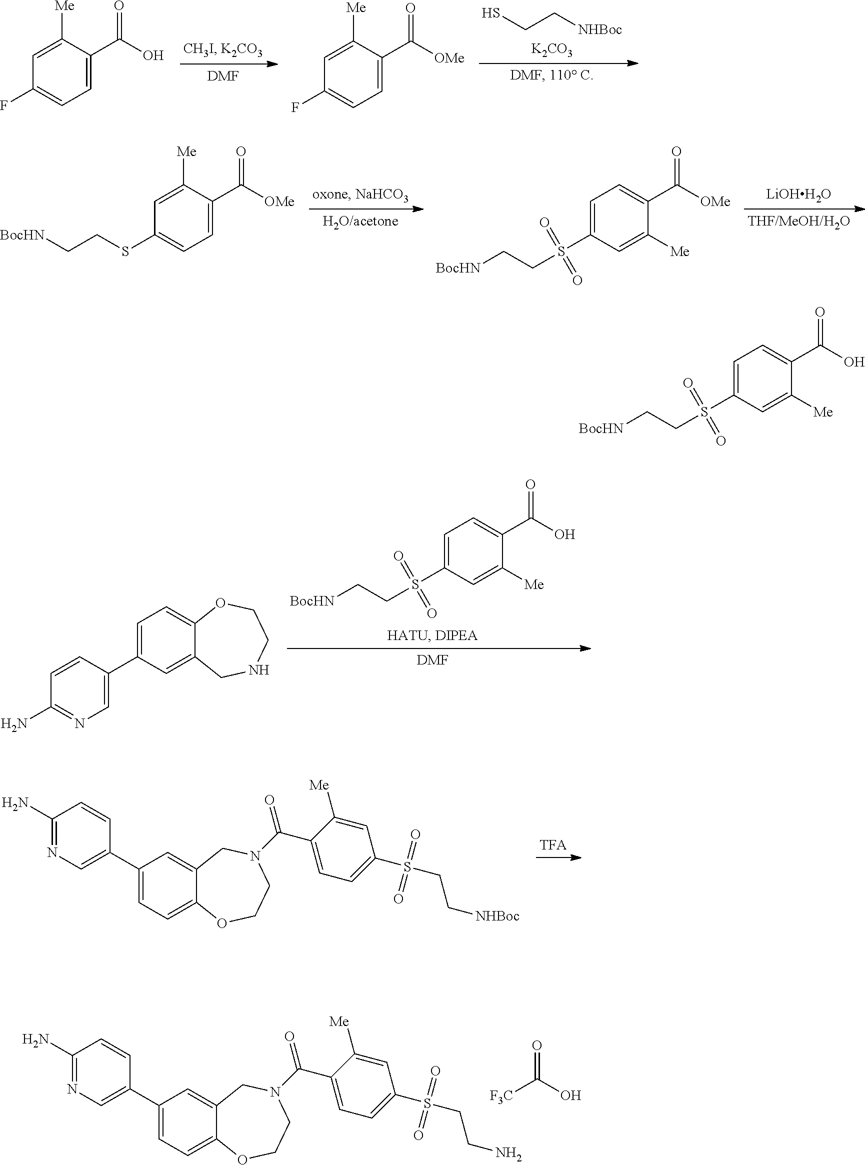

### Step 1: Synthesis of methyl 4-fluoro-2-methylbenzoate

To a solution of 4-fluoro-2-methylbenzoic acid (86 g, 557.94 mmol, 1.0 equiv) in DMF (900 mL) was added K₂CO₃ (231.33 g, 1.67 mol, 3.0 equiv) and iodomethane (79.19 g, 557.94 mmol, 34.73 mL, 1.0 equiv). The mixture was stirred at room temperature for 1 h. The solution of methyl 4-fluoro-2-methylbenzoate in DMF (900 mL) was used directly in the next step.

### Step 2: Synthesis of methyl 4-((2-((tert-butoxycarbonyl)amino)ethyl)thio)-2-methylbenzoate

To a solution of methyl 4-fluoro-2-methylbenzoate (93.8 g, 557.94 mmol, 1.0 equiv) in DMF (900 mL) was added tert-butyl (2-mercaptoethyl)carbamate (98.91 g, 557.97 mmol, 1.0 equiv) and K₂CO₃ (154.23 g, 1.12 mol, 2.0 equiv). The reaction was stirred at 110° C. for 12 h, at which point the mixture was cooled to room temperature and added to H₂O (1000 mL). The aqueous layer was then extracted with EtOAc (3×600 mL) and the combined organic layers were washed with brine, dried, and concentrated under reduced pressure. Purification by silica gel chromatography (0→25% EtOAc/petroleum ether) afforded the desired product as a colorless oil (144 g, 79% yield).

### Step 3: Synthesis of methyl 4-((2-((tert-butoxycarbonyl)amino)ethyl)sulfonyl)-2-methylbenzoate

To two separate batches containing a solution of methyl 4-((2-((tert-butoxycarbonyl)amino)ethyl)thio)-2-methylbenzoate (72 g, 221.25 mmol, 1.0 equiv), NaOH (2 M, 110.6 mL, 1.0 equiv), and NaHCO₃ (55.76 g, 663.75 mmol, 3.0 equiv) in acetone (750 mL) was added potassium peroxymonosulfate (284.28 g, 462.41 mmol, 2.1 equiv). The mixture was stirred for 12 h at room temperature, at which point the two batches were combined and then the mixture was acidified to pH 5 by addition of 1N HCl. The aqueous layer was extracted with EtOAc (3×1500 mL) and the combined organic phases were washed with brine (2×500 mL), dried, and concentrated under reduced pressure. Purification by silica gel chromatography (0→25% EtOAc/petroleum ether) afforded the desired product as a white solid (120 g, 76% yield).

### Step 4: Synthesis of 4-((2-((tert-butoxycarbonyl)amino)ethyl)sulfonyl)-2-methylbenzoic acid

To a solution of methyl 4-((2-((tert-butoxycarbonyl)amino)ethyl)sulfonyl)-2-methylbenzoate (35 g, 97.92 mmol, 1.0 equiv) in THF (200 mL). MeOH (100 mL) and H₂O (100 mL) was added LiOH·H₂O (12.33 g, 293.77 mmol, 3.0 equiv) at room temperature. The reaction mixture was stirred at 40° C. for 1 h. The mixture was then concentrated under reduced pressure to remove THF and MeOH. The aqueous phase was neutralized with 0.5N HCl and the resulting precipitate was isolated by filtration. The solid cake was washed with H₂O (3×20 mL) to afford the desired product as a white solid (25 g, 74% yield).

### Step 5: Synthesis of tert-butyl (2-((4-(7-(6-aminopyridin-3-yl)-2,3,4,5-tetrahydrobenzo[f][1,4]oxazepine-4-carbonyl)-3-methylphenyl)sulfonyl)ethyl)carbamate

To a solution of 4-((2-((tert-butoxycarbonyl)amino)ethyl)sulfonyl)-2-methylbenzoic acid (9.7 g, 28.25 mmol, 1.0 equiv) and 5-(2,3,4,5-tetrahydrobenzo[f][1,4]oxazepin-7-yl)pyridin-2-amine (8.88 g, 28.25 mmol, 1.0 equiv, 2HCl) in DMF (120 mL) was added HATU (16.11 g, 42.37 mmol, 1.5 equiv) and DIPEA (18.25 g, 141.24 mmol, 24.60 mL, 5.0 equiv). The reaction was stirred at room temperature for 1 h, at which point the reaction mixture was poured into H₂O (1000 mL). The mixture was stirred for 5 min and the resulting precipitate was collected by filtration to give the crude product. The crude product was triturated with EtOAc (100 mL), filtered, and the solid cake was dried under reduced pressure to afford the desired product as a white solid (14 g, 87% yield).

### Step 6: Synthesis of (4-((2-aminoethyl)sulfonyl)-2-methylphenyl)(7-(6-aminopyridin-3-yl)-2,3-dihydrobenzo[f][1,4]oxazepin-4(5H)-yl)methanone

A solution tert-butyl (2-((4-(7-(6-aminopyridin-3-yl)-2,3,4,5-tetrahydrobenzo[f][1,4] oxazepine-4-carbonyl)-3-methylphenyl)sulfonyl)ethyl)carbamate (19 g, 33.53 mmol, 1.0 equiv) in TFA (100 mL) was stirred at room temperature for 30 min. The solution was then concentrated under reduced pressure. The residue was triturated with MeCN (30 mL) and then dripped into MTBE (600 mL) and stirred for 20 min. The suspension was filtered and the resulting solid was dissolved in MeCN (30 mL) and concentrated under reduced pressure to afford the desired product as a light yellow solid (24 g, TFA salt). LCMS (ESI) m/z: [M+H] calcd for C₂₄H₂₆N₄O₄S: 467.18; found 467.1.

### Monomer AF. 5-(4-amino-1-((5,6,7,8-tetrahydropyrido[4,3-d]pyrimidin-2-yl)methyl)-1H-pyrazolo[3,4-d]pyrimidin-3-yl)benzo[d]oxazol-2-amine

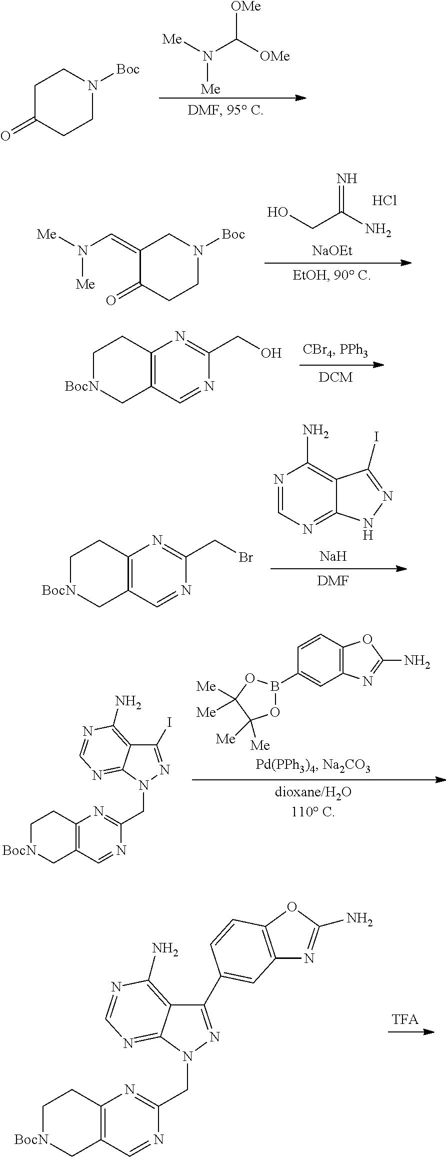

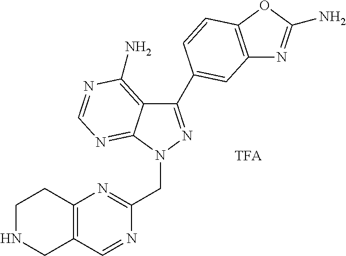 [SMILES: Nc1nc2cc(-c3nn(Cc4ncc5c(n4)CCNC5)c4ncnc(N)c34)ccc2o1]

### Step 1: Synthesis of (Z)-tert-butyl 3-((dimethylamino)methylene)-4-oxopiperidine-1-carboxylate

A solution of tert-butyl 4-oxopiperidine-1-carboxylate (15 g, 75.28 mmol, 1.0 equiv) and 1,1-dimethoxy-N,N-dimethylmethanamine (11.00 mL, 82.81 mmol, 1.1 equiv) in DMF (105 mL) was stirred at 95° C. for 12 h. The reaction mixture was then concentrated under reduced pressure and the resulting residue was dissolved in EtOAc (30 mL) and washed with brine (3×30 mL). The aqueous phase was extracted with EtOAc (50 mL), and the combined organic phases were dried and concentrated under reduced pressure to afford the desired product as a yellow solid (10.1 g, 53% yield).

### Step 2: Synthesis of tert-butyl 2-(hydroxymethyl)-7,8-dihydropyrido[4,3-d]pyrimidine-6(5H)-carboxylate

To a solution of NaOEt (1.98 g, 29.10 mmol, 1.0 equiv) in EtOH (70 mL) was added (Z)-tert-butyl 3-((dimethylamino)methylene)-4-oxopiperidine-1-carboxylate (7.4 g, 29.10 mmol, 1.0 equiv) and 2-hydroxyacetimidamide hydrochloride (3.54 g, 32.01 mmol, 1.1 equiv). The reaction mixture was heated to 90° C. for 12 h, at which point the mixture was cooled to room temperature and concentrated under reduced pressure. The residue was partitioned with EtOAc (40 mL) and washed with sat. NaHCO₃ (40 mL). The aqueous phase was extracted with EtOAc (3×20 mL) and the combined organic phases were washed with brine (2×50 mL), dried, and concentrated under reduced pressure. Purification by silica gel chromatography (25% EtOAc/petroleum ether) afforded the desired product as a yellow solid (7.24 g, 94% yield).

### Step 3: Synthesis of tert-butyl 2-(bromomethyl)-7,8-dihydropyrido[4,3-d]pyrimidine-6(5H)-carboxylate

To a solution of tert-butyl 2-(hydroxymethyl)-7,8-dihydropyrido[4,3-d]pyrimidine-6(5H)-carboxylate (6.24 g, 23.52 mmol, 1.0 equiv) and PPh₃ (12.34 g, 47.04 mmol, 2.0 equiv) in DCM (140 mL) was added CBr₄ (14.82 g, 44.69 mmol, 1.9 equiv). The mixture was stirred at room temperature for 3 h, at which point mixture was concentrated under reduced pressure. The residue was partitioned between EtOAc (20 mL) and H₂O (20 mL), the aqueous phase was extracted with EtOAc (3×20 mL). The combined organic phases were washed with brine (2×50 mL), dried, and concentrated under reduced pressure. Purification by silica gel chromatography (14% EtOAc/petroleum ether) afforded the desired product as a yellow solid (3.6 g, 47% yield).

### Step 4: Synthesis of tert-butyl 2-((4-amino-3-iodo-1H-pyrazolo[3,4-d]pyrimidin-1-yl)methyl)-7,8-dihydropyrido[4,3-d]pyrimidine-6(5H)-carboxylate

To a solution of 3-iodo-1H-pyrazolo[3,4-d]pyrimidin-4-amine (1.59 g, 6.09 mmol, 1.0 equiv) in DMF (15 mL) was added NaH (243.73 mg, 6.09 mmol, 60 wt. %, 1.0 equiv) at 0° C. The suspension was stirred for 30 min and then tert-butyl 2-(bromomethyl)-7,8-dihydropyrido[4,3-d]pyrimidine-6(5H)-carboxylate (2.2 g, 6.70 mmol, 1.1 equiv) was added. The reaction mixture was warmed to room temperature and stirred for 3 h. The mixture was poured into H₂O at 0° C. and the precipitate was collected by filtration to afford the desired product as a brown solid (2.5 g, 66% yield).

### Step 5: Synthesis of tert-butyl 2-((4-amino-3-(2-aminobenzo[d]oxazol-5-yl)-1H-pyrazolo[3,4-d]pyrimidin-1-yl)methyl)-7,8-dihydropyrido[4,3-d]pyrimidine-6(5H)-carboxylate

To a solution of tert-butyl 2-((4-amino-3-iodo-1H-pyrazolo[3,4-d]pyrimidin-1-yl)methyl)-7,8-dihydropyrido[4,3-d]pyrimidine-6(5H)-carboxylate (4.55 g, 8.95 mmol, 1.0 equiv), 5-(4,4,5,5-tetramethyl-1,3,2-dioxaborolan-2-yl)benzo[d]oxazol-2-amine (2.79 g, 10.74 mmol, 1.2 equiv) and Na₂CO₃ (4.74 g, 44.76 mmol, 5.0 equiv) in dioxane (70 mL) and H₂O (35 mL) was added Pd(PPh₃)₄ (1.03 g, 895.11 μmol, 0.1 equiv). The reaction mixture was heated to 110° C. for 3 h, at which point the mixture was cooled to room temperature and poured into H₂O at 0° C. The precipitate was filtered, and the solid cake was dried under reduced pressure. The crude product was washed with EtOAc (50 mL) to afford the desired product as light yellow solid (3.14 g, 68% yield).

### Step 6: Synthesis of 5-(4-amino-1-((5,6,7,8-tetrahydropyrido[4,3-d]pyrimidin-2-yl)methyl)-1H-pyrazolo[3,4-d]pyrimidin-3-yl)benzo[d]oxazol-2-amine

A solution of tert-butyl 2-((4-amino-3-(2-aminobenzo[d]oxazol-5-yl)-1H-pyrazolo[3,4-d]pyrimidin-1-yl)methyl)-7,8-dihydropyrido[4,3-d]pyrimidine-6(5H)-carboxylate (3.14 g, 6.10 mmol, 1.0 equiv) in TFA (20 mL) was stirred at room temperature for 30 min. The mixture was concentrated under reduced pressure and the resulting residue was added dissolved in MeCN (7 mL) and added to MTBE (700 mL). The precipitate was collected by filtration to afford the desired product as a brown solid (4.25 g, 92% yield, 3 TFA). LCMS (ESI) m/z: [M+H] calcd for C₂₀H₁₈N₁₀O: 415.18; found 415.1.

### Monomer AG. 5-(4-amino-1-((2-((2-aminoethyl)sulfonyl)-1,2,3,4-tetrahydroisoquinolin-6-yl)methyl)-1H-pyrazolo[3,4-d]pyrimidin-3-yl)benzo[d]oxazol-2-amine

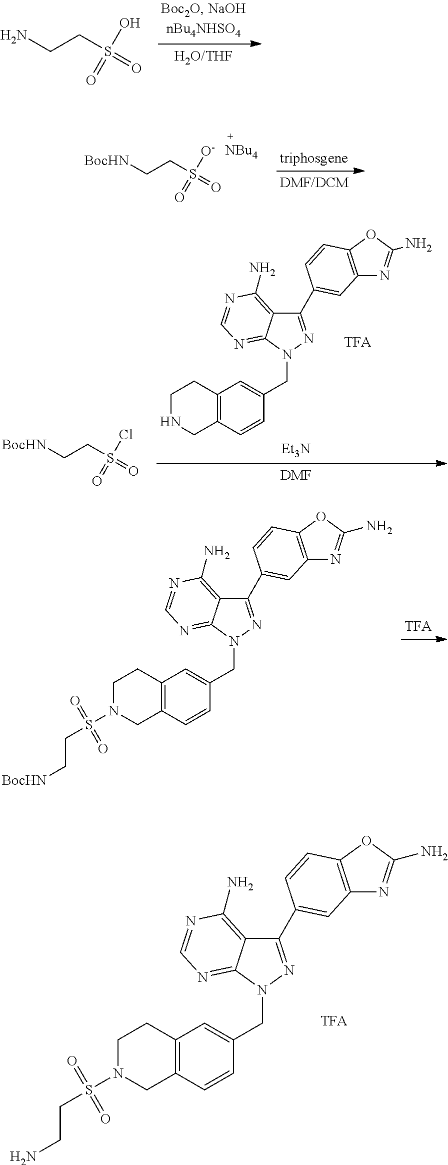

### Step 1: Synthesis of N-Boc taurine tetrabutylammonium salt

To a solution of 2-aminoethanesulfonic acid (10.00 mL, 79.91 mmol, 1.0 equiv) in THF (60 mL) and aqueous NaOH (2 M. 40 mL, 1.0 equiv) was added BoC₂O (18.31 g, 83.90 mmol, 1.05 equiv). The mixture was stirred at room temperature for 15 h, at which point the mixture was extracted with EtOAc (10 mL). The aqueous phase was diluted with H₂O (450 mL), treated with LiOH·H₂O (3.35 g, 79.83 mmol, 1.0 equiv) and nBu₄NHSO₄ (27.13 g 79.90 mmol, 1.0 equiv) and stirred for 30 min. This mixture was extracted with DCM (3×80 mL), and the combined organic phases were dried and concentrated under reduced pressure to afford the desired product as a colorless oil (34.26 g, 91% yield).

### Step 2: Synthesis of tert-butyl (2-(chlorosulfonyl)ethyl)carbamate

To a solution of N-Boc taurine tetrabutylammonium salt (4.7 g, 10.05 mmol, 1.0 equiv) in DCM (42 mL) was added DMF (77.32 μL, 1.00 mmol, 0.1 equiv) followed by a solution of triphosgene (0.5 M. 8.04 mL, 0.4 equiv) in DCM at 0° C. The mixture was warmed to room temperature and stirred for 30 min. The solution of tert-butyl (2-(chlorosulfonyl)ethyl)carbamate (2.45 g, crude) in DCM was used directly in the next step.

### Step 3: Synthesis of tert-butyl (2-((6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)sulfonyl)ethyl)carbamate

To a solution of 5-(4-amino-1-((1,2,3,4-tetrahydroisoquinolin-6-yl)methyl)-1H-pyrazolo[3,4-d]pyrimidin-3-yl)benzo[d]oxazol-2-amine (6.04 g, 9.44 mmol, 1.0 equiv, 2TFA) in DMF (40 mL) was added Et₃N (7.88 mL, 56.63 mmol, 6.0 equiv). A solution of tert-butyl (2-(chlorosulfonyl)ethyl)carbamate in DCM (42 mL) at 0° C. was added. The mixture was warmed to room temperature and stirred 16 h. The reaction mixture was concentrated under reduced pressure to remove DCM and the resulting solution was purified by reverse phase chromatography (15→45% MeCN/H₂O) to afford the desired product as a white solid (5.8 g, 83% yield, TFA). LCMS (ESI) m/z: [M+H] calcd for C₂₉H₃₃N₉O₅S: 620.24; found 620.3.

### Step 4: Synthesis of 5-(4-amino-1-((2-((2-aminoethyl)sulfonyl)-1,2,3,4-tetrahydroisoquinolin-6-yl)methyl)-1H-pyrazolo[3,4-d]pyrimidin-3-yl)benzo[d]oxazol-2-amine

A solution of tert-butyl (2-((6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)sulfonyl)ethyl)carbamate (5.8 g, 9.36 mmol, 1.0 equiv) in TFA (48 mL) was stirred at room temperature for 0.5 h, at which point the reaction mixture was concentrated under reduced pressure. The crude product dissolved in MeCN (30 mL) and was added dropwise into MTBE (200 mL). The mixture was stirred for 5 min and filtered, the filter cake was dried under reduced pressure to afford the desired product as a yellow solid (3.6 g, 62% yield, 2.2TFA). LCMS (ESI) m/z: [M+H] calcd for C₂₄H₂₅N₉O₃S: 520.19; found 520.1.

### Monomer AH. tert-butyl ((5-((4-amino-3-(2-aminobenzo[d]oxazol-5-yl)-1H-pyrazolo[3,4-d]pyrimidin-1-yl)methyl)pyrimidin-2-yl)methyl)carbamate

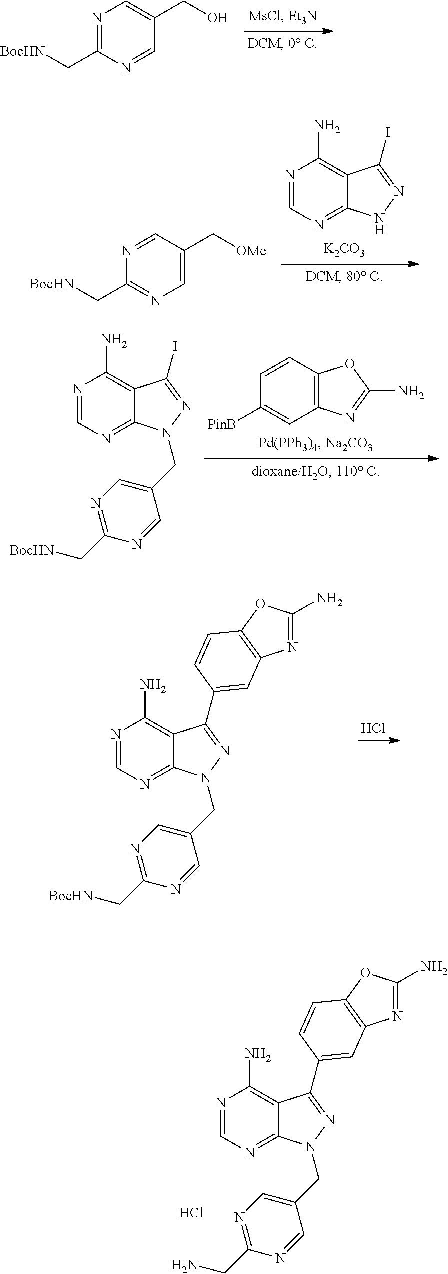

### Step 1: Synthesis of (2-(((tert-butoxycarbonyl)amino)methyl)pyrimidin-5-yl)methyl methanesulfonate

To a solution of tert-butyl ((5-(hydroxymethyl)pyrimidin-2-yl)methyl)carbamate (4.2 g, 17.55 mmol, 1.0 equiv) in DCM (42 mL) at 0° C. was added Et₃N (7.33 mL, 52.66 mmol, 3.0 equiv) followed by MsCl (2.41 g, 21.06 mmol, 1.63 mL, 1.2 equiv). The mixture was stirred at 0° C. for 10 min, and then H₂O (15 mL) was added. The reaction mixture was extracted with DCM (5×10 mL) and the combined organic phases were washed with brine (5 mL), dried, filtered, and concentrated under reduced pressure to afford the desired product (5.5 g, 98.7% yield) as a colorless solid.

### Step 2: Synthesis of tert-butyl ((5-((4-amino-3-iodo-1H-pyrazolo[3,4-d]pyrimidin-1-yl)methyl)pyrimidin-2-yl)methyl)carbamate

To a solution of (2-(((tert-butoxycarbonyl)amino)methyl)pyrimidin-5-yl)methyl methanesulfonate (5.47 g, 17.24 mmol, 1.2 equiv) and 3-iodo-1H-pyrazolo[3,4-d]pyrimidin-4-amine (3.75 g, 14.37 mmol, 1.0 equiv) in DMF (55 mL) at room temperature was added K₂CO₃ (5.96 g, 43.10 mmol, 3 equiv). The mixture was stirred at 80° C. for 5 h, at which point H₂O (100 mL) and brine (20 mL) were poured into the reaction mixture. The solution was extracted with EtOAc (10×30 mL) and the combined organic phases were dried, filtered, and concentrated under reduced pressure. Purification by silica gel chromatography (0→30% EtOAc/MeOH) afforded the desired product (2 g, 28.9% yield) as a yellow solid.

### Step 3: Synthesis of tert-butyl ((5-((4-amino-3-(2-aminobenzo[d]oxazol-5-yl)-1H-pyrazolo[3,4-d]pyrimidin-1-yl)methyl)pyrimidin-2-yl)methyl)carbamate

To a solution of tert-butyl ((5-((4-amino-3-iodo-1H-pyrazolo[3,4-d]pyrimidin-1-yl)methyl)pyrimidin-2-yl)methyl)carbamate (2 g, 4.15 mmol, 1.0 equiv), 5-(4,4,5,5-tetramethyl-1,3,2-dioxaborolan-2-yl)-1,3-benzoxazol-2-amine (1.13 g, 4.35 mmol, 1.05 equiv) and Na₂CO₃ (688.39 mg, 8.29 mmol, 2.0 equiv) in dioxane (20 mL) and H₂O (10 mL) was added Pd(PPh₃)₄ (479.21 mg, 414.70 μmol, 0.1 equiv). The mixture was stirred at 110° C. for 1 h, at which time the mixture was cooled to room temperature, filtered, and the solid cake washed with MeOH (3×10 mL). The filtrate was concentrated under reduced pressure to remove MeOH and then added dropwise into H₂O (50 mL). The resulting suspension was filtered, and the filter cake was washed with H₂O (3×10 mL). The solid cake was stirred in MeOH (20 mL) for 30 min. The resulting suspension was filtered, and the filter cake washed with MeOH (3×8 mL). The filter cake was dried under reduced pressure to afford the desired product (1.03 g, 48.9% yield) as a white solid. LCMS (ESI) m/z: [M+H] calcd for C₂₃H₂₄N₁₀O₃: 489.21; found 489.2.

### Step 4: Synthesis of 5-(4-amino-1-{[2-(aminomethyl)pyrimidin-5-yl]methyl}-1H-pyrazolo[3,4-d]pyrimidin-3-yl)-1,3-benzoxazol-2-amine

To tert-butyl ((5-((4-amino-3-(2-aminobenzo[d]oxazol-5-yl)-1H-pyrazolo[3,4-d]pyrimidin-1-yl)methyl)pyrimidin-2-yl)methyl)carbamate (100 mg, 0.205 mmol, 1.0 equiv) was added con. HCl (850 μL, 10.2 mmol, 50 equiv). The reaction was stirred for 1 h and was then poured into acetone (3 mL). The resulting precipitate was filtered, washed with acetone, and dried under reduced pressure to afford the desired product (80 mg, 92% yield) as a brown solid. LCMS (ESI) m/z: [M+H] calcd for C₁₈H₁₆N₁₀O: 389.16; found 389.0.

### Monomer AI. 5-(4-(dimethylamino)-1-((1,2,3,4-tetrahydroisoquinolin-6-yl)methyl)-1H-pyrazolo[3,4-d]pyrimidin-3-yl)benzo[d]oxazol-2-amine trifluoroacetic acid salt

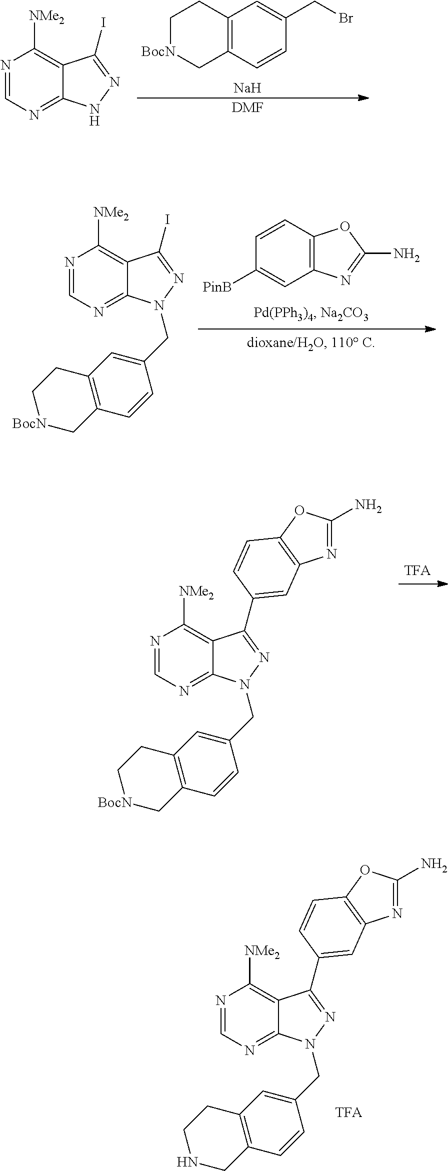

### Step 1: Synthesis of tert-butyl 6-((4-(dimethylamino)-3-iodo-1H-pyrazolo[3,4-d]pyrimidin-1-yl)methyl)-3,4-dihydroisoquinoline-2(1H)-carboxylate

To a solution of 3-iodo-N,N-dimethyl-1H-pyrazolo[3,4-d]pyrimidin-4-amine (3.6 g, 12.45 mmol, 1.0 equiv) in DMF (36 mL) at 0° C. was added NaH (523.00 mg, 13.08 mmol, 60 wt. %, 1.05 equiv). The mixture was stirred at 0° C. for 30 min. To the reaction mixture was then added a solution of tert-butyl 6-(bromomethyl)-3,4-dihydroisoquinoline-2(1H)-carboxylate (4.47 g, 13.70 mmol, 1.1 equiv) in DMF (18 mL) at 0° C. The mixture was stirred at room temperature for 2 h. The reaction mixture was then added to cold H₂O (200 mL) and stirred for 30 min. The resulting precipitate was collected by filtration to afford the desired product (6 g, 71.9% yield) as a white solid.

### Step 2: Synthesis of tert-butyl 6-((3-(2-aminobenzo[d]oxazol-5-yl)-4-(dimethylamino)-1H-pyrazolo[3,4-d]pyrimidin-1-yl)methyl)-3,4-dihydroisoquinoline-2(1H)-carboxylate

To a solution of tert-butyl 6-((4-(dimethylamino)-3-iodo-1H-pyrazolo[3,4-d]pyrimidin-1-yl)methyl)-3,4-dihydroisoquinoline-2(1H)-carboxylate (2 g, 2.96 mmol, 1.0 equiv) and 5-(4,4,5,5-tetramethyl-1,3,2-dioxaborolan-2-yl)benzo[d]oxazol-2-amine (922.81 mg, 3.55 mmol, 1.2 equiv) in dioxane (24 mL) and H₂O (12 mL) was added Na₂CO₃ (1.57 g, 14.78 mmol, 5.0 equiv) and Pd(PPh₃)₄ (341.66 mg, 295.66 μmol, 0.1 equiv). The mixture was stirred at 110° C. for 12 h. The reaction mixture was then poured into cold H₂O (200 mL) and stirred for 30 min. The resulting precipitate was collected by filtration. Purification by silica gel chromatography (5→100% petroleum ether/EtOAc) afforded the desired product (1.2 g, 72.3% yield) as a yellow solid.

### Step 3: Synthesis of 5-(4-(dimethylamino)-1-((1,2,3,4-tetrahydroisoquinolin-6-yl)methyl)-1H-pyrazolo[3,4-d]pyrimidin-3-yl)benzo[d]oxazol-2-amine

A solution of tert-butyl 6-((3-(2-aminobenzo[d]oxazol-5-yl)-4-(dimethylamino)-1H-pyrazolo[3,4-d]pyrimidin-1-yl)methyl)-3,4-dihydroisoquinoline-2(1H)-carboxylate (1.7 g, 3.14 mmol, 1.0 equiv) in TFA (10 mL) was stirred at room temperature for 30 min. The reaction mixture was then concentrated under reduced pressure. The residue was added to MeCN (10 mL) and the solution was added dropwise into MTBE (200 mL). The resulting solid was dissolved in MeCN (30 mL) and the solution was concentrated under reduced pressure to afford the desired product (1.67 g, 92.9% yield.) as a yellow solid. LCMS (ESI) m/z: [M+H] calcd for C₂₄H₂₄N₈O: 441.22; found 441.2.

### Monomer AJ. 4-amino-5-(2-aminobenzo[d]oxazol-5-yl)-5H-pyrimido[5,4-b]indole-7-carboxylic acid

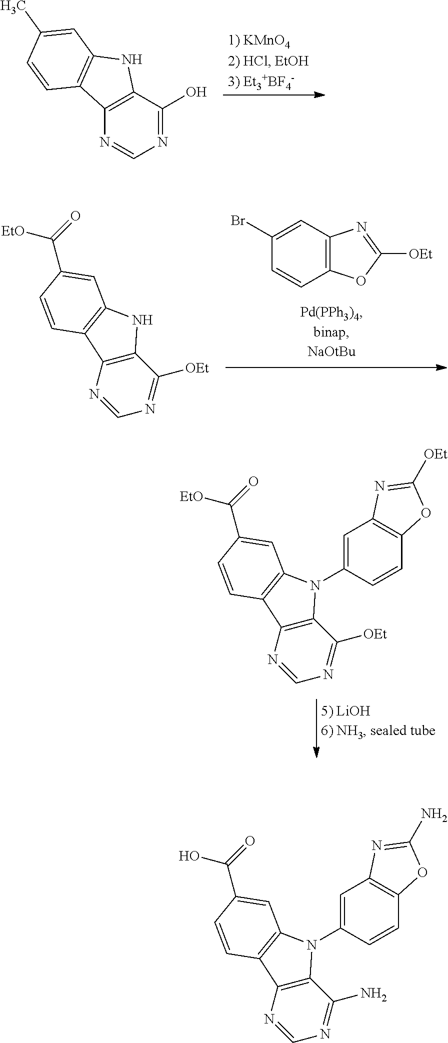 [SMILES: Cc1ccc2c(c1)[nH]c1c(O)ncnc12 ; CCOC(=O)c1ccc2c(c1)[nH]c1c(OCC)ncnc12 ; CCOc1nc2cc(Br)ccc2o1 ; CCOC(=O)c1ccc2c3ncnc(OCC)c3n(-c3ccc4oc(OCC)nc4c3)c2c1 ; Nc1nc2cc(-n3c4cc(C(=O)O)ccc4c4ncnc(N)c43)ccc2o1]

This monomer can be prepared from 7-methyl-5H-pyrimido[5,4-b]indol-4-ol by benzylic oxidation to the carboxylic acid, conversion to the ethyl ester, followed by O-ethylation with triethyloxonium tetrafluoroboroate. Palladium-mediated arylation followed by ester hydrolysis and final ammonia-olysis provides the monomer.

### Monomer AK. 4-amino-5-(2-aminobenzo[d]oxazo-5-yl)-5H-pyrimido[5,4-b]indole-8-carboxylic acid

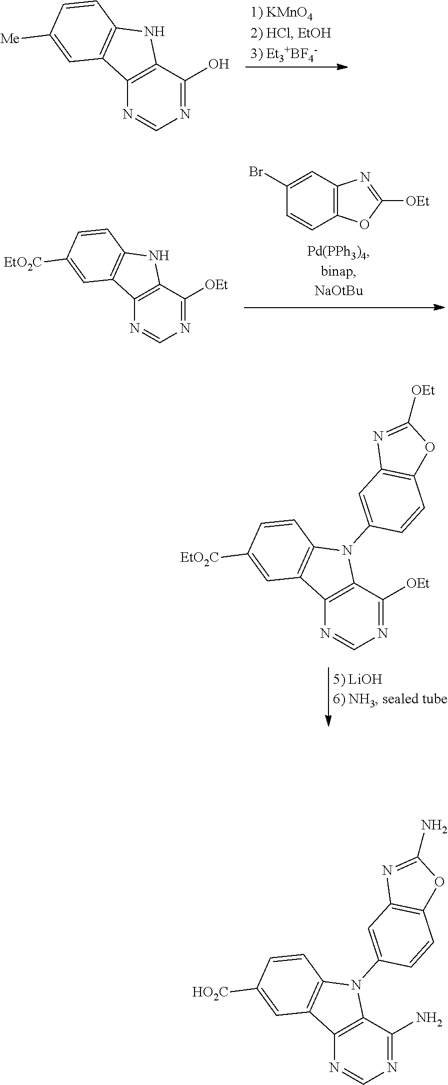 [SMILES: Cc1ccc2[nH]c3c(O)ncnc3c2c1 ; CCOC(=O)c1ccc2[nH]c3c(OCC)ncnc3c2c1 ; CCOc1nc2cc(Br)ccc2o1 ; CCOC(=O)c1ccc2c(c1)c1ncnc(OCC)c1n2-c1ccc2oc(OCC)nc2c1 ; Nc1nc2cc(-n3c4ccc(C(=O)O)cc4c4ncnc(N)c43)ccc2o1]

This monomer can be prepared following a similar route as that to prepare the previous monomer, but using the isomeric starting material from 8-methyl-5H-pyrimido[5,4-b]indol-4-ol. Benzylic oxidation to the carboxylic acid, conversion to the ethyl ester, followed by O-ethylation with triethyloxonium tetrafluoroboroate and palladium-mediated arylation, followed by ester hydrolysis and final ammonia-olysis provides the monomer.

### Monomer AL. 3-(2,4-bis((S)-3-methylmorpholino)-4a,8a-dihydropyrido[2,3-d]pyrimidin-7-yl)benzoic acid

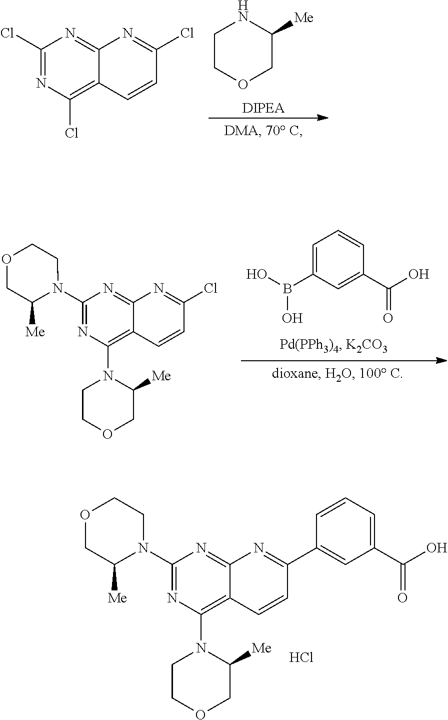

### Step 1: Synthesis of (3S)-4-[7-chloro-2-[(3S)-3-methylmorpholin-4-yl]pyrido[2,3-d]pyrimidin-4-yl]3-methyl-morpholine

To a solution of 2,4,7-trichloropyrido[2,3-d]pyrimidine (4.0 g, 17.06 mmol, 1.0 equiv) in DMA (10 mL) was added (3S)-3-methylmorpholine (4.31 g, 42.65 mmol, 2.5 equiv) and DIPEA (5.51 g, 42.65 mmol, 7.43 mL, 2.5 equiv). The reaction solution was heated to 70° C. for 48 h. The reaction suspension was cooled to room temperature, poured into cold H₂O (50 mL) to precipitate out a solid. The solid was filtered and the filter cake was rinsed with H₂O, and dried under reduced pressure to give the crude product, which was purified by column chromatography on silica gel (0→100% petroleum ether/EtOAc) to give (3S)-4-[7-chloro-2-[(3S)-3-methylmorpholin-4-yl]pyrido[2,3-d]pyrimidin-4-yl] 3-methyl-morpholine (3.5 g, 56.4% yield) as a yellow solid. LCMS (ESI) m/z: [M+H] calcd for C₁₇H₂₂ClN₅O₂: 364.15; found 364.2.

### Step 2: Synthesis of 3-[2,4-bis[(3S)-3-methylmorpholin-4-yl]pyrido[2,3-d]pyrimidin-7-yl]benzoic acid

To a solution of (3S)-4-[7-chloro-2-[(3S)-3-methylmorpholin-4-yl]pyrido[2,3-d]pyrimidin-4-yl]-3-methyl-morpholine (2 g, 5.50 mmol, 1.0 equiv) and 3-boronobenzoic acid (1.09 g, 6.60 mmol, 1.2 equiv) in 1,4-dioxane (40 mL) was added a solution of K₂CO₃ (911.65 mg, 6.60 mmol, 1.2 equiv) in H₂O (4 mL), followed by Pd(PPh₃)₄ (317.60 mg. 274.85 μmol, 0.05 equiv). The solution was degassed for 10 min and refilled with N₂, then the reaction mixture was heated to 100° C. under N₂ for 5 h. The reaction was cooled to room temperature and filtered. The filtrate was acidified by HCl (2N) to pH 3, and the aqueous layer was washed with EtOAc (3×20 mL). The aqueous phase was concentrated under reduced pressure to give a residue, which was purified by column chromatography on silica gel (50%→100% petroleum ether/EtOAc) to give 3-[2,4-bis[(3S)-3-methylmorpholin-4-yl]pyrido[2,3-d]pyrimidin-7-yl]benzoic acid hydrochloride (2.5 g, 89.9% yield) as a yellow solid. LCMS (ESI) m/z: [M+H] calcd for C₂₄H₂₇N₅O₄: 450.21; found 450.2.

Reference for preparation of this monomer: Menear, K.; Smith, G. C. M.; Malagu, K.; Duggan, H. M. E.; Martin, N. M. B.; Leroux, F. G. M. 2012. Pyrido-, pyrazo- and pyrimido-pyrimidine derivatives as mTOR inhibitors. U.S. Pat. No. 8,101,602. Kudos Pharmaceuticals, Ltd, which is incorporated by reference in its entirety.

### Monomer AM. (1r,4r)-4-[4-amino-5-(7-methoxy-1H-indol-2-yl)imidazo[4,3-f][1,2,4]triazin-7-yl]cyclohexane-1-carboxylic acid

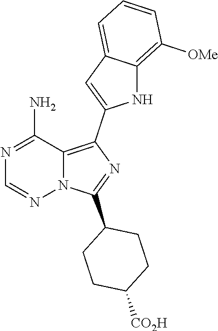 [SMILES: COc1cccc2cc(-c3nc([C@H]4CC[C@H](C(=O)O)CC4)n4ncnc(N)c34)[nH]c12]

This monomer, also known as OSI-027 (CAS #=936890-98-1), is a commercially available compound. At the time this application was prepared, it was available for purchase from several vendors.

### Monomer AN. 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)pyrimidine-5-carboxylic acid

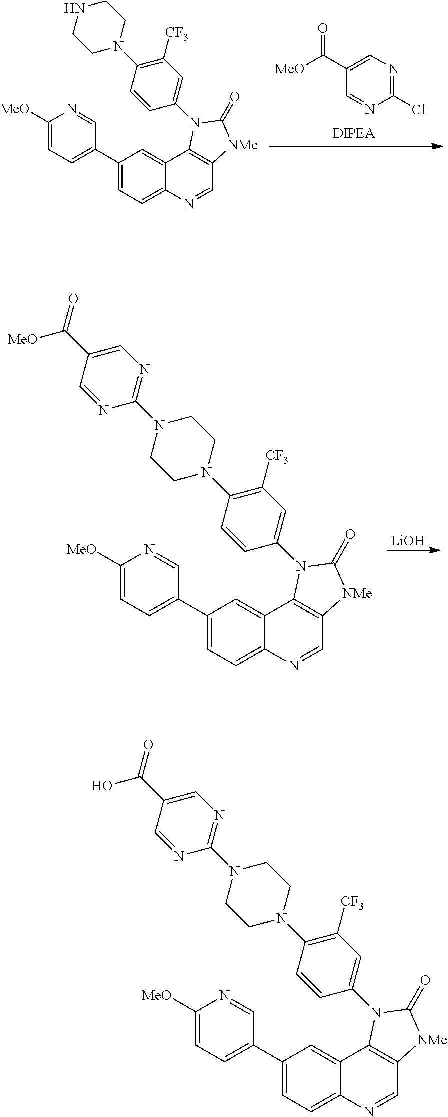

Preparation of this monomer proceeds by reaction of BGT226 with methyl 2-chloropyrimidine-5-carboxylate, followed by ester hydrolysis, to give the titled Monomer.

### Monomer AO. 4-amino-5-{1H-pyrrolo[2,3-b]pyridin-5-yl}-5H-pyrimido[5,4-b]indole-8-carboxylic acid

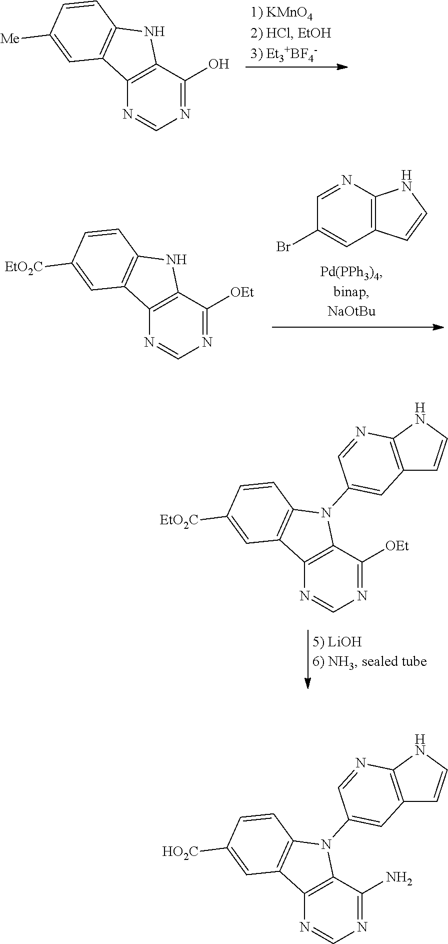 [SMILES: Cc1ccc2[nH]c3c(O)ncnc3c2c1 ; CCOC(=O)c1ccc2[nH]c3c(OCC)ncnc3c2c1 ; Brc1cnc2[nH]ccc2c1 ; CCOC(=O)c1ccc2c(c1)c1ncnc(OCC)c1n2-c1cnc2[nH]ccc2c1 ; Nc1ncnc2c3cc(C(=O)O)ccc3n(-c3cnc4[nH]ccc4c3)c12]

This monomer can be prepared from 7-methyl-5H-pyrimido[5,4-b]indol-4-ol by benzylic oxidation to the carboxylic acid, conversion to the ethyl ester, followed by O-ethylation with triethyloxonium tetrafluoroboroate. Palladium-mediated arylation followed by ester hydrolysis and final ammonia-olysis provides the monomer.

### Preparation of Pre- and Post-Linkers

### Building block A. tert-butyl N-[(tert-butoxy)carbonyl]-N-{[2-(piperazin-1-yl)pyrimidin-5-yl]methyl}carbamate

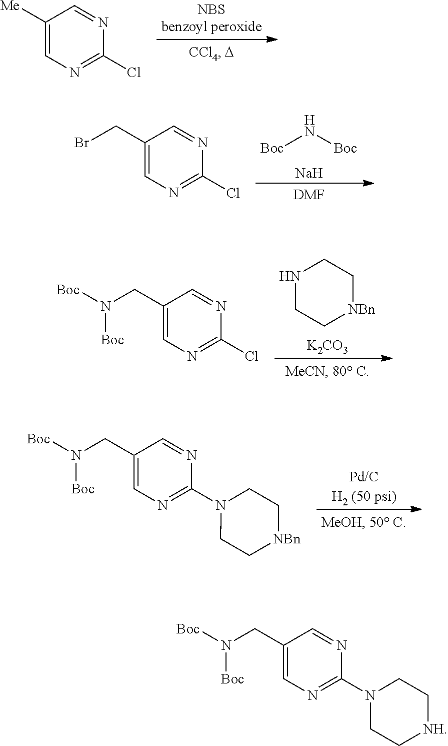

### Step 1: Synthesis of 5-(bromomethyl)-2-chloropyrimidine

To a solution of 2-chloro-5-methylpyrimidine (92 g, 715.62 mmol, 1.0 equiv) in CCl₄ (1000 mL) was added NBS (178.31 g, 1.00 mol, 1.4 equiv) and benzoyl peroxide (3.47 g, 14.31 mmol, 0.02 equiv). The mixture was stirred at 76° C. for 18 h. The reaction mixture was then cooled to room temperature and concentrated under reduced pressure. The reaction mixture was filtered and the solid cake was washed with DCM (150 mL). The resulting solution was concentrated under reduced pressure to give the crude product. The residue was purified by silica gel chromatography (1/0 to 0/1 petroleum ether/EtOAc) to give the product (70.8 g, 47.7% crude yield) as yellow oil, which was used directly for the next step. LCMS (ESI) m/z: [M+H] calcd for C₅H₄BrClN₂: 206.93; found 206.9.

### Step 2: Synthesis of tert-butyl N-tert-butoxycarbonyl-N-((2-piperazin-1-ylpyrimidin-5-yl)methyl)carbamate

To a solution of tert-butyl N-tert-butoxycarbonylcarbamate (36.89 g, 169.79 mmol, 0.74 equiv) in DMF (750 mL) was added NaH (6.88 g, 172.09 mmol, 60 wt. %, 0.75 equiv) at 0° C. The mixture was stirred at 0° C. for 30 min. Then, 5-(bromomethyl)-2-chloro-pyrimidine (47.6 g, 229.45 mmol, 1.0 equiv) was added at 0° C. The reaction mixture was stirred at room temperature for 15.5 h. The mixture was then poured into H₂O (1600 mL) and the aqueous phase was extracted with EtOAc (3×300 mL). The combined organic phase was washed with brine (2×200 mL), dried with anhydrous Na₂SO₄, filtered and concentrated under reduced pressure. The residue was purified by silica gel chromatography (1/0 to 0/1 petroleum ether/EtOAc) to give the product (70 g, crude) as a yellow solid, which was used to next step directly.

### Step 3: Synthesis of tert-butyl N-tert-butoxycarbonyl-N-[(2-piperazin-1-ylpyrimidin-5-yl)methyl]carbamate

To a solution of 1-benzylpiperazine (30.44 g, 122.16 mmol, 1.0 equiv, 2HCl) in MeCN (550 mL) was added tert-butyl N-tert-butoxycarbonyl-N-((2-chloropyrimidin-5-yl)methyl)carbamate (42 g, 122.16 mmol, 1.0 equiv) and K₂CO₃ (84.42 g, 610.81 mmol, 5.0 equiv). The mixture was stirred at 80° C. for 61 h. The reaction mixture was then diluted with EtOAc (150 mL) and the mixture was filtered. The resulting solution was concentrated under reduced pressure to give the crude product. The residue was purified by silica gel chromatography (1/0 to 0/1 petroleum ether/EtOAc) to give the product (45 g, 74% yield) as a white solid.

### Step 4: Synthesis of tert-butyl N-tert-butoxycarbonyl-N-[(2-piperazin-1-ylpyrimidin-5-yl)methyl]carbamate

To a solution of tert-butyl N-[[2-(4-benzylpiperazin-1-yl)pyrimidin-5-yl]methyl]-N-tert-butoxycarbonyl-carbamate (24 g, 49.63 mmol, 1.0 equiv) in MeOH (600 mL) was added Pd/C (24 g, 47.56 mmol, 10 wt. %, 1.0 equiv) under argon. The mixture was degassed under reduced pressure and purged with H₂ three times. The mixture was stirred under H₂ (50 psi) at 50° C. for 19 h. The reaction mixture was cooled to room temperature, filtered, and the filter cake was washed with MeOH (500 mL). The resulting solution was concentrated under reduced pressure. The residue was purified by silica gel chromatography (1/0 to 0/1 EtOAc/MeOH) to give the product (25.5 g, 68% yield) as a white solid.

### Building Block B. 2-(4-(5-(((tert-butoxycarbonyl)amino)methyl)pyrimidin-2-yl)piperazin-1-yl)pyrimidine-5-carboxylic acid

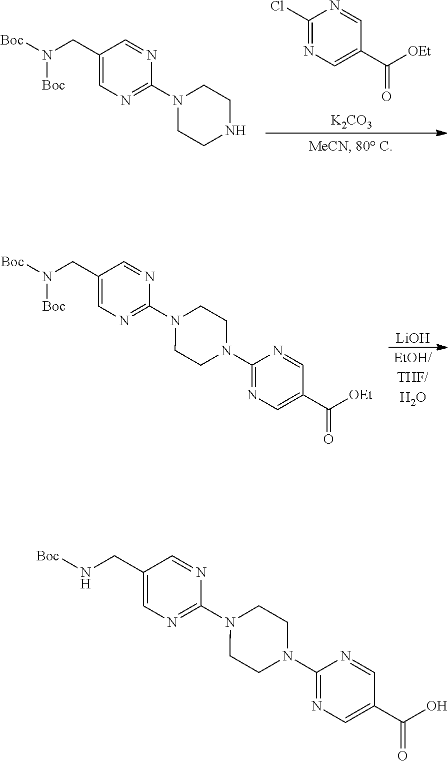

### Step 1: Synthesis of ethyl 2-(4-(5-((bis(tert-butoxycarbonyl)amino)methyl)pyrimidin-2-yl)piperazin-1-yl)pyrimidine-5-carboxylate

To a solution of ethyl 2-chloropyrimidine-5-carboxylate (2.37 g, 12.71 mmol, 1.0 equiv) and tert-butyl N-tert-butoxycarbonyl-N-((2-piperazin-1-ylpyrimidin-5-yl)methyl)carbamate (5 g, 12.71 mmol, 1.0 equiv) in MeCN (80 mL) was added K₂CO₃ (5.27 g, 38.12 mmol, 3.0 equiv). The mixture was stirred at 80° C. for 16 h. The reaction mixture was then poured into H₂O (200 mL) and the suspension was filtered. The filtrate was washed with H₂O (80 mL) and dried under reduced pressure to give the product (6.1 g, 87% yield) as a white solid.

### Step 2: Synthesis of 2-(4-(5-(((tert-butoxycarbonyl)amino)methyl)pyrimidin-2-yl)piperazin-1-yl)pyrimidine-5-carboxylic acid

To a solution of ethyl 2-(4-(5-((bis(tert-butoxycarbonyl)amino)methyl)pyrimidin-2-yl)piperazin-1-yl)pyrimidine-5-carboxylate (5 g, 9.20 mmol, 1.0 equiv) in H₂O (50 mL), EtOH (15 mL) and THF (50 mL) was added LiOH·H₂O (1.54 g, 36.79 mmol, 4.0 equiv). The reaction mixture was stirred at 55° C. for 16 h. The mixture was then concentrated to remove THF and EtOH and then the mixture was diluted with H₂O (55 mL) and was acidified (pH=3) with aqueous HCl (1 N). The mixture was filtered and the filter cake was washed with H₂O (36 mL). The filter cake was dried under reduced pressure to give the product (2.7 g, 69.3%) as a white solid. LCMS (ESI) m/z: [M+H] calcd for C₁₉H₂₅N₇O₄: 416.21; found 416.1.

### Building Block C. tert-butyl 2-(piperazin-1-yl)-7,8-dihydropyrido[4,3-d]pyrimidine-6(5H)-carboxylate

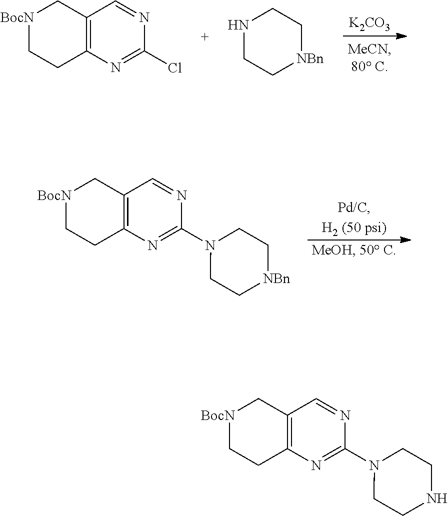

### Step 1: Synthesis of tert-butyl 2-(4-benzylpiperazin-1-yl)-7,8-dihydropyrido[4,3-d]pyrimidine-6(5H)-carboxylate

To a solution of tert-butyl 2-chloro-7,8-dihydropyrido[4,3-d]pyrimidine-6(5H)-carboxylate (15 g, 55.61 mmol, 1.0 equiv) in MeCN (150 mL) was added 1-benzylpiperazine (11.76 g, 66.73 mmol, 1.2 equiv) and K₂CO₃ (46.12 g, 333.67 mmol, 6.0 equiv). The mixture was stirred at 80° C. for 27 h. The reaction mixture was diluted with EtOAc (200 mL), filtered and concentrated under reduced pressure. The crude product was purified by silica gel chromatography (1/0 to 0/1 petroleum ether/EtOAc) to give the product (20.2 g, 80% yield) as a white solid. LCMS (ESI) m/z: [M+H] calcd for C₂₃H₃₁N₅O₂: 410.26; found 410.1.

### Step 2: Synthesis of tert-butyl 2-(piperazin-1-yl)-7,8-dihydropyrido[4,3-d]pyrimidine-6(5H)-carboxylate

To a solution of tert-butyl 2-(4-benzylpiperazin-1-yl)-7,8-dihydropyrido[4,3-d]pyrimidine-6(5H)-carboxylate (8 g, 19.53 mmol, 1.0 equiv) in MeOH (200 mL) was added Pd/C (8 g, 19.53 mmol, 10 wt. %, 1.0 equiv) under argon. The mixture was degassed and purged with H₂ three times. The mixture was stirred under H₂ (50 psi) at 50° C. for 19 h. The reaction mixture was cooled to room temperature, filtered through a pad of Celite and the filter cake was washed with MeOH (150 mL). The resulting solution was concentrated under reduced pressure. The crude product was washed with petroleum ether (60 mL) to give the product (9.25 g, 72% yield) as a white solid. LCMS (ESI) m/z: [M+H] calcd for C₁₆H₂₅N₅O₂: 320.21; found 320.2.

### Building Block D. 2-(4-(6-(tert-butoxycarbonyl)-5,6,7,8-tetrahydropyrido[4,3-d]pyrimidin-2-yl)piperazin-1-yl)pyrimidine-5-carboxylic acid

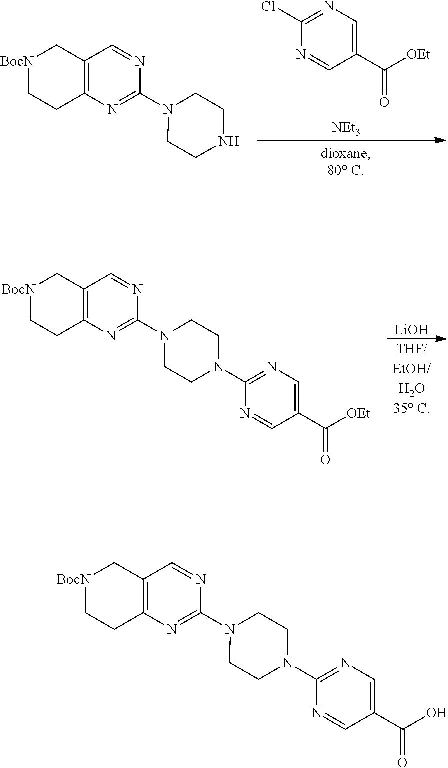

### Step 1: Synthesis of tert-butyl 2-(4-(5-(ethoxycarbonyl)pyrimidin-2-yl)piperazin-1-yl)-7,8-dihydropyrido[4,3-d]pyrimidine-6(5H)-carboxylate

To a solution of ethyl 2-chloropyrimidine-5-carboxylate (4.09 g, 21.92 mmol, 1.0 equiv) in dioxane (80 mL) was added tert-butyl 2-(piperazin-1-yl)-7,8-dihydropyrido[4,3-d]pyrimidine-6(5H)-carboxylate (7 g, 21.92 mmol, 1.0 equiv) and Et₃N (9.15 mL, 65.75 mmol, 3.0 equiv). The mixture was stirred at 90° C. for 64 h. The solution was poured into H₂O (200 mL) and then the mixture was filtered and the filter cake was washed with H₂O (100 mL) followed by petroleum ether (60 mL). The filter cake was dried under reduced pressure to give the product (10.1 g, 92% yield) as a brown solid. LCMS (ESI) m/z: [M+H] calcd for C₂₃H₃₁N₇O₄: 470.25; found 470.4.

### Step 2: Synthesis of 2-(4-(6-(tert-butoxycarbonyl)-5,6,7,8-tetrahydropyrido[4,3-d]pyrimidin-2-yl)piperazin-1-yl)pyrimidine-5-carboxylic acid

To a solution of tert-butyl 2-(4-(5-(ethoxycarbonyl)pyrimidin-2-yl)piperazin-1-yl)-7,8-dihydropyrido[4,3-d]pyrimidine-6(5H)-carboxylate (6.0 g, 12.78 mmol, 1.0 equiv) in THF (40 mL), EtOH (20 mL) and H₂O (40 mL) was added LiOH·H₂O (1.07 g, 25.56 mmol, 2.0 equiv). The reaction mixture was stirred at 35° C. for 15 h. The mixture was then concentrated under reduced pressure to remove THF and EtOH. The mixture was then diluted with H₂O (500 mL) and was adjusted to pH 3 with aqueous HCl (1 N). The mixture was filtered and the filter cake was washed with H₂O (80 mL) followed by petroleum ether (80 mL). The filter cake was dried under reduced pressure to give the product (3.8 g, 65% yield) as a white solid. LCMS (ESI) m/z: [M+H] calcd for C₂₁H₂₇N₇O₄: 442.22; found 442.3.

### Building Block E. tert-butyl methyl((2-(piperazin-1-yl)pyrimidin-5-yl)methyl)carbamate

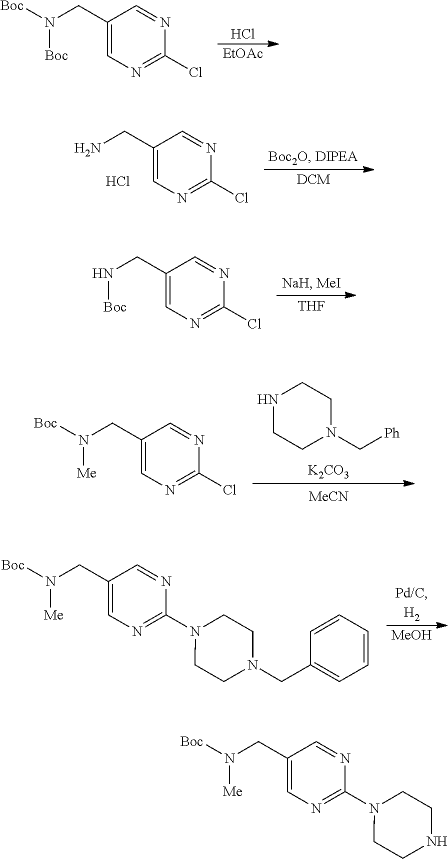

### Step 1: Synthesis of (2-chloropyrimidin-5-yl)methanamine

To a solution of tert-butyl N-tert-butoxycarbonyl-N-((2-chloropyrimidin-5-yl)methyl)carbamate (28 g, 81.44 mmol, 1.0 equiv) in EtOAc (30 mL) was added HCl in EtOAc (260 mL). The reaction mixture was stirred at room temperature for 5 h. The reaction mixture was filtered and the filter cake was washed with EtOAc (100 mL). The solid cake was dried under reduced pressure to give the product (14.3 g, 96.6% yield. HCl) as a white solid.

### Step 2: Synthesis of tert-butyl ((2-chloropyrimidin-5-yl)methyl)carbamate

To a solution of (2-chloropyrimidin-5-yl)methanamine (13 g, 72.21 mmol, 1.0 equiv, HCl) in DCM (130 mL) was added DIPEA (20.41 mL, 144.42 mmol, 1.8 equiv) and Boc₂O (16.59 mL, 72.21 mmol, 1.0 equiv), then the mixture was stirred at room temperature for 3 h. The reaction mixture was added to H₂O (100 mL) and then the aqueous layer was separated and extracted with DCM (2×100 mL). Then combined organic phase was washed with sat. NH₄Cl (2×200 mL) and brine (2×200 mL), dried with anhydrous Na₂SO₄, filtered and concentrated under reduced pressure. The residue was purified by silica gel chromatography (1/0 to 1/1 petroleum ether/EtOAc) to give the product (12 g, 68.2% yield) as a white solid.

### Step 3: Synthesis of tert-butyl ((2-chloropyrimidin-5-yl)methyl)(methyl)carbamate

To a solution of tert-butyl ((2-chloropyrimidin-5-yl)methyl)carbamate (11 g, 45.14 mmol, 1.0 equiv) and Mel (14.05 mL, 225.70 mmol, 5.0 equiv) in THF (150 mL) was added NaH (1.99 g, 49.65 mmol, 60 wt. %, 1.1 equiv) at 0° C. The mixture was stirred at 0° C. for 3 h and then the reaction was quenched with H₂O (100 mL). The aqueous phase was extracted with EtOAc (3×150 mL) and the combined organic phase was washed with brine (50 mL), dried with anhydrous Na₂SO₄, filtered and concentrated under reduced pressure. The residue was purified by silica gel chromatography (1/0 to 3/1 petroleum ether/EtOAc) to give the product (9 g, 77.4% yield) as a white solid.

### Step 4: Synthesis of tert-butyl ((2-(4-benzylpiperazin-1-yl)pyrimidin-5-yl)methyl)(methyl)carbamate

To a solution of tert-butyl ((2-chloropyrimidin-5-yl)methyl)(methyl)carbamate (9 g, 34.92 mmol, 1.0 equiv) in MeCN (90 mL) was added 1-benzylpiperazine (8.70 g, 34.92 mmol, 1.0 equiv, 2HCl), and K₂CO₃ (24.13 g, 174.61 mmol, 5.0 equiv). The reaction mixture was stirred at 80° C. for 20 h. The mixture was then filtered and concentrated under reduced pressure. The residue was purified by silica gel chromatography (1/0 to 1/1 petroleum ether/EtOAc) to give the product (12 g, 86.4% yield) as a yellow oil.

### Step 5: Synthesis of tert-butyl methyl((2-(piperazin-1-yl)pyrimidin-5-yl)methyl)carbamate

To a solution of tert-butyl ((2-(4-benzylpiperazin-1-yl)pyrimidin-5-yl)methyl)(methyl)carbamate (12 g, 30.19 mmol, 1.0 equiv) in MeOH (120 mL) was added Pd/C (2 g, 10 wt. %). The suspension was degassed and purged with H₂ and then the mixture was stirred under H₂ (15 psi) at room temperature for 3 h. The reaction mixture was filtered through Celite and the filtrate was concentrated under reduced pressure. The residue was purified by silica gel chromatography 1/0 to 1/1 petroleum ether/EtOAc) to give semi-pure material (9 g) as a yellow oil. Petroleum ether was added to the residue and the solution was stirred at −60° C. until solid appeared. The suspension was filtered and the filtrate was concentrated under reduced pressure to give the product (4.07 g, 55.6% yield) as a yellow oil. LCMS (ESI) m/z: [M+H] calcd for C₁₅H₂₅N₅O₂: 308.21; found 308.1.

### Building Block F. 2-(4-(5-(((tert-butoxycarbonyl)(methyl)amino)methyl) pyrimidin-2-yl)piperazin-1-yl)pyrimidine-5-carboxylic acid

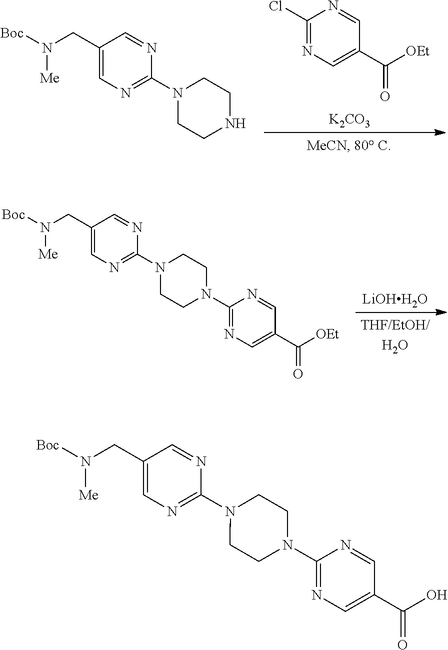 [SMILES: CN(Cc1cnc(N2CCNCC2)nc1)C(=O)OC(C)(C)C ; "K2CO3" ; CCOC(=O)c1cnc(Cl)nc1 ; CCOC(=O)c1cnc(N2CCN(c3ncc(CN(C)C(=O)OC(C)(C)C)cn3)CC2)nc1 ; "LiOH·H2O" ; CN(Cc1cnc(N2CCN(c3ncc(C(=O)O)cn3)CC2)nc1)C(=O)OC(C)(C)C]

### Step 1: Synthesis of ethyl 2-(4-(5-(((tert-butoxycarbonyl)(methyl)amino)methyl) pyrimidin-2-yl)piperazin-1-yl)pyrimidine-5-carboxylate

To a mixture of tert-butyl methyl((2-(piperazin-1-yl)pyrimidin-5-yl)methyl)carbamate (4.3 g, 13.99 mmol, 1.0 equiv) and ethyl 2-chloropyrimidine-5-carboxylate (2.87 g, 15.39 mmol, 1.1 equiv) in MeCN (20 mL) was added K₂CO₃ (3.87 g, 27.98 mmol, 2.0 equiv). The mixture was stirred at 80° C. for 12 h. The reaction mixture then cooled to room temperature and was filtered. The filtrate was concentrated under reduced pressure and the crude product was purified by silica gel chromatography (1/0 to 1/1 petroleum ether/EtOAc) to give the product (4.7 g, 71.3% yield) as a white solid.

### Step 2: Synthesis of 2-(4-(5-(((tert-butoxycarbonyl)(methyl)amino)methyl) pyrimidin-2-yl)piperazin-1-yl)pyrimidine-5-carboxylic acid

To a solution of ethyl 2-(4-(5-(((tert-butoxycarbonyl)(methyl)amino)methyl)pyrimidin-2-yl)piperazin-1-yl)pyrimidine-5-carboxylate (6 g, 13.11 mmol, 1.0 equiv) in THF (100 mL), EtOH (30 mL), and H₂O (30 mL) was added LiOH·H₂O (1.10 g, 26.23 mmol, 2.0 equiv). The mixture was stirred at room temperature for 16 h. The mixture was then concentrated under reduced pressure to remove THF and EtOH and then neutralized by the addition of 1N HCl. The resulting precipitate was collected by filtration to give the product (5.11 g, 90.1% yield) as a white solid. LCMS (ESI) m/z: [M+H] calcd for C₂₀H₂₇N₇O₄: 430.22; found 430.2.

### Building Block G. tert-butyl N-tert-butoxycarbonyl-N-((2-(2-((tert-butyl(diphenyl)silyl)oxymethyl)piperazin-1-yl)pyrimidin-5-yl)methyl)carbamate

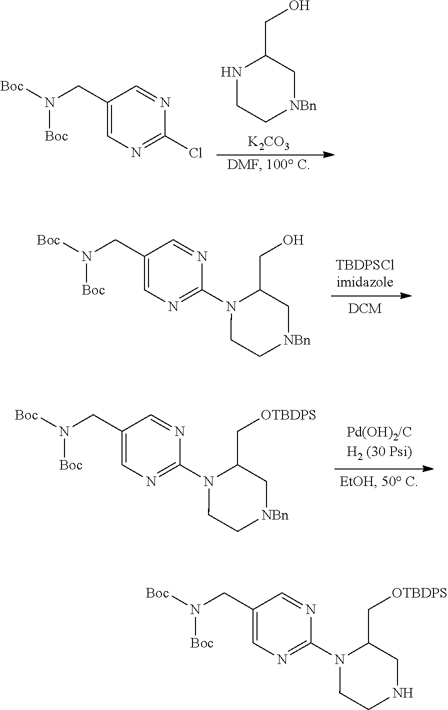

### Step 1: Synthesis of tert-butyl N-((2-(4-benzyl-2-(hydroxymethyl)piperazin-1-yl)pyrimidin-5-yl)methyl)-N-tert-butoxycarbonyl-carbamate

To a solution of tert-butyl N-tert-butoxycarbonyl-N-((2-chloropyrimidin-5-yl)methyl)carbamate (18.33 g, 53.32 mmol, 1.1 equiv) and (4-benzylpiperazin-2-yl)methanol (10 g, 48.48 mmol, 1.0 equiv) in DMF (100 mL) was added K₂CO₃ (13.40 g, 96.95 mmol, 2.0 equiv). The mixture was stirred at 100° C. for 12 h. The reaction mixture was then cooled to room temperature and H₂O (100 mL) was added. The aqueous layer was extracted with EtOAc (2×150 mL) and the combined organic layer was washed with brine (20 mL), dried with Na₂SO₄, filtered and the filtrate was concentrated under reduced pressure to give the product (7.3 g, 29.3% yield) as a yellow oil. LCMS (ESI) m/z: [M+H] calcd for C₂₇H₃₉N₅O₅: 514.31; found 514.5.

### Step 2: Synthesis of tert-butyl N-((2-(4-benzyl-2-((tert-butyl(diphenyl)silyl)oxymethyl)piperazin-1-yl)pyrimidin-5-yl)methyl)-N-tert-butoxycarbonyl-carbamate

To a solution of tert-butyl N-((2-(4-benzyl-2-(hydroxymethyl)piperazin-1-yl)pyrimidin-5-yl)methyl)-N-tert-butoxycarbonyl-carbamate (2.3 g, 4.48 mmol, 1.0 equiv) in DCM (30 mL) was added imidazole (609.69 mg, 8.96 mmol, 2.0 equiv) and TBDPSCl (1.73 mL, 6.72 mmol, 1.5 equiv). The reaction mixture was stirred at room temperature for 2 h. The mixture was then washed with H₂O (100 mL) and the aqueous phase extracted with EtOAc (2×60 mL). The combined organic phase was washed with brine (20 mL), dried with Na₂SO₄, filtered and concentrated under reduced pressure. The residue was purified by silica gel chromatography (20/1 to 3/1 petroleum ether/EtOAc) to give the product (4 g, 59.4% yield) as a yellow oil. LCMS (ESI) m/z: [M+H] calcd for C₄₃H₅₇N₅O₅Si: 752.42; found 752.4.

### Step 3: Synthesis of tert-butyl N-tert-butoxycarbonyl-N-((2-(2-((tert-butyl(diphenyl)silyl)oxymethyl)piperazin-1-yl)pyrimidin-5-yl)methyl)carbamate

To a solution of tert-butyl N-((2-(4-benzyl-2-((tert-butyl(diphenyl)silyl)oxymethyl)piperazin-1-yl)pyrimidin-5-yl)methyl)-N-tert-butoxycarbonyl-carbamate (3.3 g, 4.39 mmol, 1.0 equiv) in EtOH (10 mL) was added Pd(OH)₂/C (1 g, 10 wt. %). The mixture was heated to 50° C. under H₂ (30 psi) for 30 h. The mixture was then cooled to room temperature, filtered through Celite, and concentrated under reduced pressure. The residue was purified by silica gel chromatography (20/1 to 3/1 EtOAc/EtOH) to give the product (1.44 g, 45.6% yield) as a yellow solid. LCMS (ESI) m/z: [M+H] calcd for C₃₆H₅₁N₅O₅Si: 662.38; found 662.3.

### Building Block H. 2-(4-(5-(((tert-butoxycarbonyl)amino)methyl)pyrimidin-2-yl)-3-(hydroxymethyl)piperazin-1-yl)pyrimidine-5-carboxylic acid

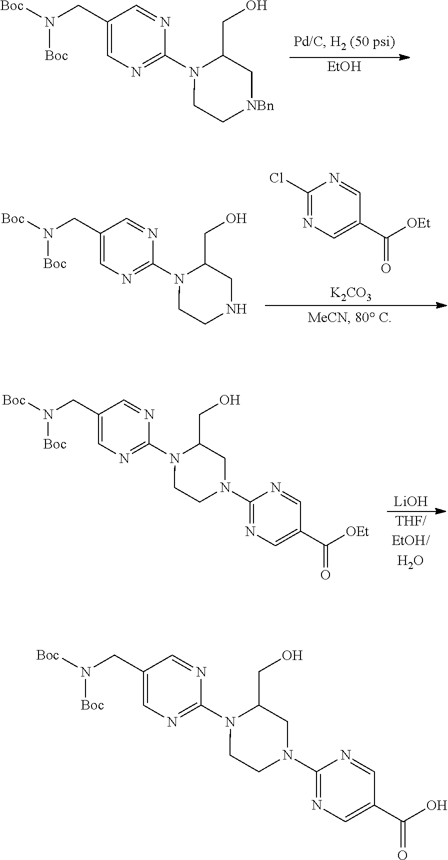

### Step 1: Synthesis of tert-butyl N-tert-butoxycarbonyl-N-((2-(2-(hydroxymethyl)piperazin-1-yl) pyrimidin-5-yl)methyl)carbamate

To a solution of tert-butyl N-((2-(4-benzyl-2-(hydroxymethyl)piperazin-1-yl)pyrimidin-5-yl)methyl)-N-tert-butoxycarbonyl-carbamate (3 g, 5.84 mmol, 1.0 equiv) in EtOH (40 mL) was added Pd/C (2 g, 10 wt. %). The suspension was degassed and purged with H₂, then stirred under H₂ (50 psi) at 30° C. for 16 h. The reaction mixture was cooled to room temperature and filtered through Celite and then concentrated under reduced pressure to give the product (1.6 g, crude) as a yellow oil. LCMS (ESI) m/z: [M+H] calcd for C₂₀H₃₃N₅O₅: 424.26; found 424.3.

### Step 2: Synthesis of ethyl 2-(4-(5-((bis(tert-butoxycarbonyl)amino)methyl)pyrimidin-2-yl)-3-(hydroxymethyl)piperazin-1-yl)pyrimidine-5-carboxylate

To a solution of tert-butyl N-tert-butoxycarbonyl-N-((2-(2-(hydroxymethyl)piperazin-1-yl)pyrimidin-5-yl)methyl)carbamate (1.4 g, 3.31 mmol, 1.0 equiv) in MeCN (20 mL) was added K₂CO₃ (2.28 g, 16.53 mmol, 5.0 equiv) and ethyl 2-chloropyrimidine-5-carboxylate (616.84 mg, 3.31 mmol, 1.0 equiv). The solution was stirred at 80° C. for 4 h. The mixture was cooled to room temperature and poured into H₂O (30 mL). The aqueous layer was extracted with EtOAc (2×30 mL) and the combined organic layer was washed with brine (20 mL), dried with Na₂SO₄, filtered and concentrated under reduced pressure. The mixture was purified by silica gel chromatography (20/1 to 3/1 petroleum ether/EtOAc) to give the product (1.6 g, 66.7% yield) as a light yellow solid. LCMS (ESI) m/z: [M+H] calcd for C₂₇H₃₉N₇O₇: 574.30; found 574.4.

### Step 3: Synthesis of 2-(4-(5-(((tert-butoxycarbonyl)amino)methyl)pyrimidin-2-yl)-3-(hydroxymethyl)piperazin-1-yl)pyrimidine-5-carboxylic acid

To a solution of ethyl 2-(4-(5-((bis(tert-butoxycarbonyl)amino)methyl)pyrimidin-2-yl)-3-(hydroxymethyl)piperazin-1-yl)pyrimidine-5-carboxylate (1.4 g, 2.44 mmol, 1.0 equiv) in THF (6 mL) and EtOH (6 mL) at 0° C. was added a solution of LiOH·H₂O (512.07 mg, 12.20 mmol, 5.0 equiv) in H₂O (3 mL). The reaction mixture was warmed to room temperature and stirred for 2 h. The mixture was then concentrated under reduced pressure to remove THF and EtOH. The aqueous phase was adjusted to pH 3 with 0.1 M HCl and the resulting suspension was filtered. The solid cake was dried under reduced pressure to give the product (613.14 mg, 55.6% yield) as a white solid. LCMS (ESI) m/z: [M+H] calcd for C₂₀H₂₇N₇O₅: 446.22; found 446.2.

### Building Block I. tert-butyl N-[(tert-butoxy)carbonyl]-N-({2-[(3R)-3-(hydroxymethyl)piperazin-1-yl]pyrimidin-5-yl}methyl)carbamate

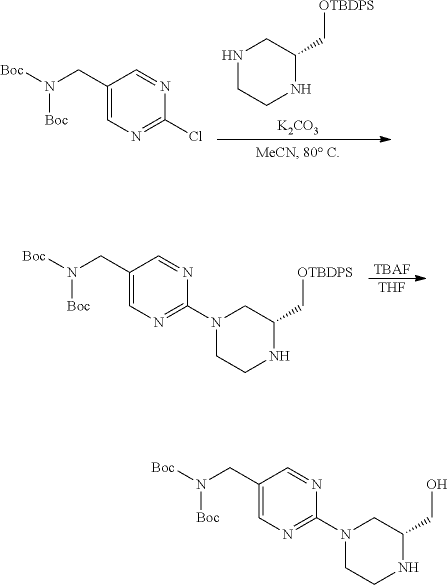

### Step 1: Synthesis of (R)-tert-butyl-N-tert-butoxycarbonyl-((2-(3-(((tert-butyldiphenylsilyl)-oxy)methyl)piperazin-1-yl)pyrimidin-5-yl)methyl)carbamate

To a solution of tert-butyl-N-tert-butoxycarbonyl-((2-chloropyrimidin-5-yl)methyl)carbamate (24.24 g, 70.51 mmol, 1.0 equiv) in MeCN (300 mL) was added (R)-2-(((tert-butyldiphenylsilyl)oxy)methyl)piperazine (25 g, 70.51 mmol, 1.0 equiv) and K₂CO₃ (29.24 g, 211.53 mmol, 3.0 equiv). The mixture was stirred at 80° C. for 16 h. The reaction mixture was then cooled to room temperature, diluted with EtOAc (200 mL), filtered and the filtrate was concentrated under reduced pressure. Purification by silica gel chromatography (0→100% EtOAc/petroleum ether) afforded the desired product (46.5 g, 94% yield) as a white solid.

Step 2: Synthesis of tert-butyl N-[(tert-butoxy)carbonyl]-N-({2-[(3R)-3-(hydroxymethyl)piperazin-1-yl]pyrimidin-5-yl}methyl)carbamate

To a solution of (R)-tert-butyl-N-tert-butoxycarbonyl-((2-(3-(((tert-butyldiphenylsilyl)oxy)methyl)piperazin-1-yl)pyrimidin-5-yl)methyl)carbamate (12 g, 18.13 mmol, 1.0 equiv) in THF (120 mL) was added TBAF (1 M, 23.93 mL, 1.3 equiv). The mixture was stirred at room temperature for 2 h. The reaction mixture was then poured into H₂O (300 mL) and the aqueous phase was extracted with EtOAc (3×80 mL). The combined organic phases were combined, washed with brine (80 mL), dried, filtered and the filtrate was concentrated under reduced pressure. Purification by silica gel chromatography (0→20% MeOH/DCM) afforded the desired product (5 g, 64% yield) as a yellow solid.

### Building Block J. 2-{4-[5-({[(tert-butoxy)carbonyl]amino}methyl)pyrimidin-2-yl]piperazin-1-yl}pyrimidine-5-carboxylic acid

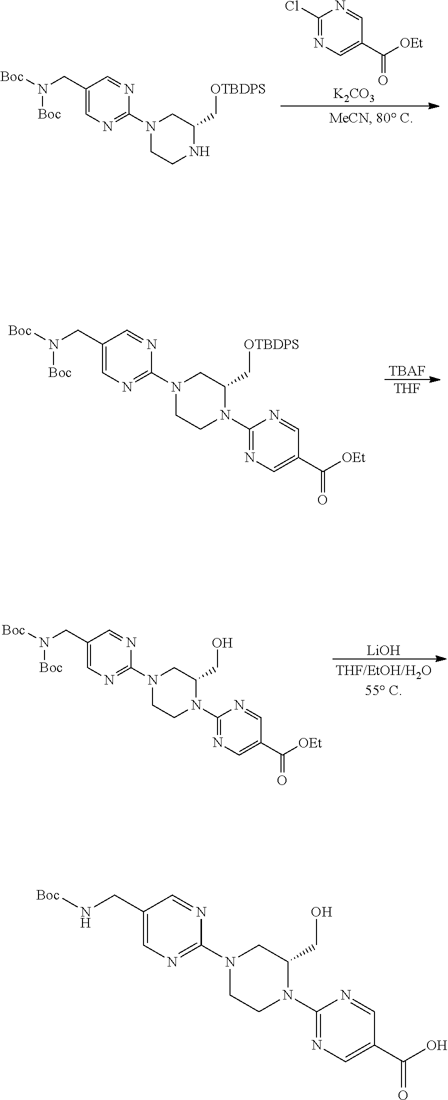

### Step 1: Synthesis of (R)-ethyl 2-(4-(5-(((di-tert-butoxycarbonyl)amino)methyl)pyrimidin-2-yl)-2-(((tert-butyldiphenylsilyl)oxy)methyl)piperazin-1-yl)pyrimidine-5-carboxylate

To a solution of (R)-tert-butyl-N-tert-butoxycarbonyl-N-((2-(3-(((tert-butyldiphenylsilyl)oxy)methyl)piperazin-1-yl)pyrimidin-5-yl)methyl)carbamate (31.5 g, 45.21 mmol, 1.0 equiv) in MeCN (350 mL) was added ethyl 2-chloropyrimidine-5-carboxylate (8.44 g, 45.21 mmol, 1.0 equiv) and K₂CO₃ (18.75 g, 135.63 mmol, 3.0 equiv). The mixture was stirred at 80° C. for 16 h. The reaction mixture was then cooled to room temperature, diluted with EtOAc (150 mL), and filtered to remove inorganic salts. The filtrate was then concentrated under reduced pressure. Purification by silica gel chromatography (0→100% EtOAc/petroleum ether) afforded the desired product (33.5 g, 89% yield).

### Step 2: Synthesis of (R)-ethyl 2-(4-(5-(((di-tert-butoxycarbonyl)amino)methyl)pyrimidin-2-yl)-2-(hydroxymethyl)piperazin-1-yl)pyrimidine-5-carboxylate

To a solution of (R)-ethyl 2-(4-(5-(((di-tert-butoxycarbonyl)amino)methyl)pyrimidin-2-yl)-2-(((tert-butyldiphenylsilyl)oxy)methyl)piperazin-1-yl)pyrimidine-5-carboxylate (36.5 g, 44.95 mmol, 1.0 equiv) in THF (300 mL) was added TBAF (1 M, 59.33 mL, 1.32 equiv). The mixture was stirred at room temperature for 6 h, at which point the reaction mixture was poured into H₂O (500 mL). The aqueous phase was separated and extracted with EtOAc (3×150 mL) and the combined organic layers were washed with brine (150 mL), dried, filtered, and the filtrate was concentrated under reduced pressure. Purification by silica gel chromatography (0→100% EtOAc/petroleum ether) afforded the desired product (17 g, 64% yield) as a yellow oil.

### Step 3: Synthesis of (R)-2-(4-(5-(((tert-butoxycarbonyl)amino)methyl)pyrimidin-2-yl)-2-(hydroxymethyl)piperazin-1-yl)pyrimidine-5-carboxylic acid

To a solution of (R)-ethyl 2-(4-(5-(((di-tert-butoxycarbonyl)amino)methyl)pyrimidin-2-yl)-2-(hydroxymethyl)piperazin-1-yl)pyrimidine-5-carboxylate (17 g, 29.64 mmol, 1.0 equiv) in H₂O (160 mL), EtOH (80 mL) and THF (160 mL) was added LiOH·H₂O (4.97 g, 118.54 mmol, 4.0 equiv). The reaction mixture was stirred at 55° C. for 16 h. To the mixture was then added LiOH·H₂O (1.01 g, 24.00 mmol, 0.81 equiv) and the reaction mixture was stirred at 55° C. for an additional 9 h. The mixture was cooled to room temperature, diluted with H₂O (150 mL), and concentrated under reduced pressure to remove THF and EtOH. The mixture was acidified (pH=5) with 1 N HCl, filtered, and the filter cake washed with H₂O (2×30 mL). The filter cake was dried under reduced pressure to afford the desired product (9.2 g, 67% yield) as a white solid. LCMS (ESI) m/z: [M+H] calcd for C₂₀H₂₇N₇O₅: 446.22; found 446.1.

### Building Block K. tert-butyl N-[(tert-butoxy)carbonyl]-N-({2-[(3S)-3-(hydroxymethyl)piperazin-1-yl]pyrimidin-5-yl}methyl)carbamate

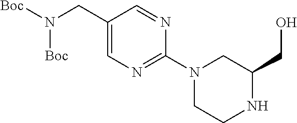 [SMILES: CC(C)(C)OC(=O)N(Cc1cnc(N2CCN[C@H](CO)C2)nc1)C(=O)OC(C)(C)C]

This building block is prepared by a process similar to that for Building block I by utilizing [(2S)-piperazin-2-yl]methanol.

### Building Block L. 2-[(2S)-4-[5-({[(tert-butoxy)carbonyl]amino}methyl)pyrimidin-2-yl]-2-(hydroxymethyl)piperazin-1-yl]pyrimidine-5-carboxylic acid

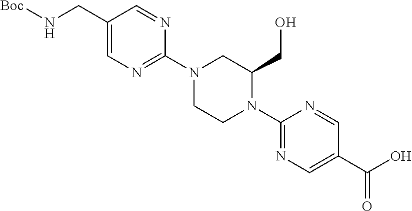 [SMILES: CC(C)(C)OC(=O)NCc1cnc(N2CCN(c3ncc(C(=O)O)cn3)[C@H](CO)C2)nc1]

This building block is prepared from Building block K by a process similar to that for Building block J.

### Building Block M. tert-butyl 2-[(3R)-3-(hydroxymethyl)piperazin-1-yl]-5H,6H,7H,8H-pyrido[4,3-d]pyrimidine-6-carboxylate

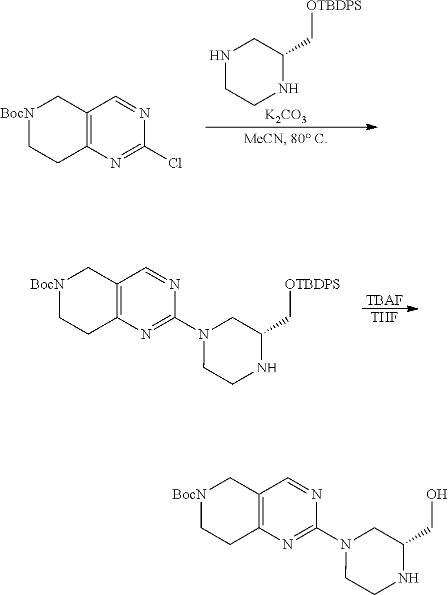

### Step 1: Synthesis of (R)-tert-butyl 2-(3-(((tert-butyldiphenylsilyl)oxy)-methyl)piperazin-1-yl)-7,8-dihydropyrido[4,3-d]pyrimidine-6(5H)-carboxylate

To a solution of (R)-2-(((tert-butyldiphenylsilyl)oxy)methyl)piperazine (25 g, 70.51 mmol, 1.0 equiv) in MeCN (250 mL) was added K₂CO₃ (29.24 g, 211.53 mmol, 3.0 equiv) and tert-butyl 2-chloro-7,8-dihydropyrido[4,3-d]pyrimidine-6(5H)-carboxylate (17.12 g, 63.46 mmol, 0.9 equiv). The mixture was stirred at 80° C. for 17 h. The reaction mixture was then cooled to room temperature, filtered, and the filtrated was concentrated under reduced pressure. Purification by silica gel chromatography (0→00% EtOAc/petroleum ether) afforded the desired product (31 g, 73.5% yield) as a white solid. LCMS (ESI) m/z: [M+H] calcd for C₃₃H₄₅N₅O₃Si: 588.34; found 588.2.

### Step 2: Synthesis of (R)-tert-butyl 2-(3-(hydroxymethyl)piperazin-1-yl)-7,8-dihydropyrido[4,3-d]pyrimidine-6(5H)-carboxylate

To a mixture of (R)-tert-butyl 2-(3-(((tert-butyldiphenylsilyl)oxy)methyl)piperazin-1-yl)-7,8-dihydropyrido[4,3-d]pyrimidine-6(5H)-carboxylate (12 g, 20.41 mmol, 1.0 equiv) in THF (120 mL) was added TBAF (1.0 M. 24.50 mL, 1.2 equiv). The mixture was stirred at room temperature for 5 h. The mixture was poured into H₂O (100 mL), and the aqueous phase was extracted with EtOAc (2×100 mL). The combined organic phases were washed with brine (100 mL), dried, filtered, and the filtrate was concentrated under reduced pressure. Purification by silica gel chromatography (0→10% MeOH/DCM) afforded the desired product (6 g, 84.1% yield) as a white solid. LCMS (ESI) m/z: [M+H] calcd for C₁₇H₂₇N₅O₃: 350.22; found 350.2.

### Building Block N. 2-[(2R)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

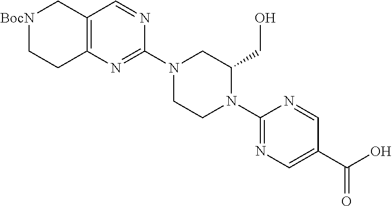 [SMILES: CC(C)(C)OC(=O)N1CCc2nc(N3CCN(c4ncc(C(=O)O)cn4)[C@@H](CO)C3)ncc2C1]

This building block is prepared from Building block M by a process similar to that for Building block J.

### Building Block O. tert-butyl 2-[(3S)-3-(hydroxymethyl)piperazin-1-yl]-5H,6H,7H,8H-pyrido[4,3-d]pyrimidine-6-carboxylate

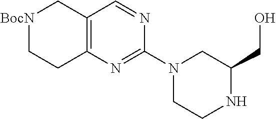 [SMILES: CC(C)(C)OC(=O)N1CCc2nc(N3CCN[C@H](CO)C3)ncc2C1]

This building block is prepared by a process similar to that for Building block I by utilizing tert-butyl 2-chloro-7,8-dihydropyrido[4,3-d]pyrimidine-6(5H)-carboxylate and [(2S)-piperazin-2-yl]methanol.

### Building Block P. 2-[(2S)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

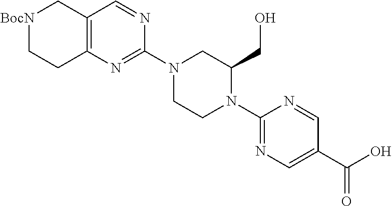 [SMILES: CC(C)(C)OC(=O)N1CCc2nc(N3CCN(c4ncc(C(=O)O)cn4)[C@H](CO)C3)ncc2C1]

This building block is prepared from Building block O by a process similar to that for Building block J.

### Building Block Q. tert-butyl N-[(tert-butoxy)carbonyl]-N-({2-[(3S)-3-[(dimethylamino)methyl]piperazin-1-yl]pyrimidin-5-yl}methyl)carbamate

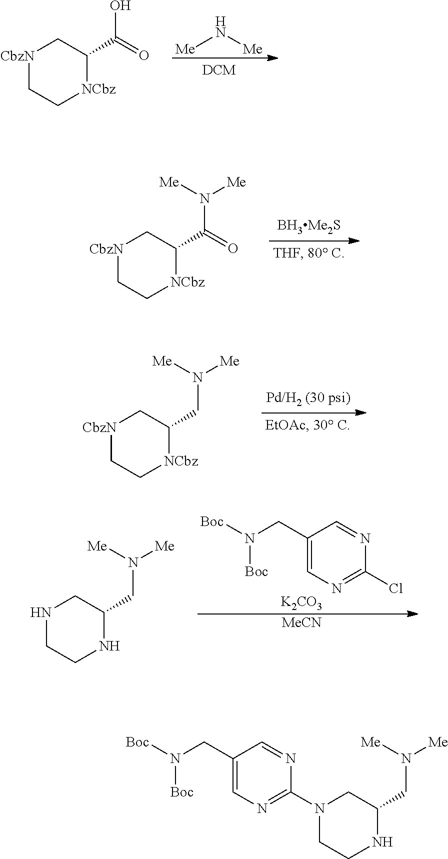

### Step 1: Synthesis of (R)-dibenzyl 2-(dimethylcarbamoyl)piperazine-1,4-dicarboxylate

To a solution of CDI (12.21 g, 75.30 mmol, 1.2 equiv) in DCM (300 mL) at 0° C. was added (R)-1,4-bis((benzyloxy)carbonyl)piperazine-2-carboxylic acid (25 g, 62.75 mmol, 1.0 equiv). The mixture was stirred at 0° C. for 0.5 h, at which time dimethylamine (8.51 mL. 92.87 mmol, 1.5 equiv, HCl) was added. The reaction mixture was warmed to room temperature and stirred for 12 h. The reaction mixture was then added to H₂O (200 mL), and the aqueous layer was separated and extracted with DCM (2×200 mL). The combined organic phases were washed with brine (2×50 mL), dried, filtered, and the filtrate was concentrated under reduced pressure. Purification by silica gel chromatography (50→100% EtOAc/petroleum ether) afforded the desired product (23.5 g, 88.0% yield) as a yellow oil.

### Step 2: Synthesis of (S)-dibenzyl 2-((dimethylamino)methyl)piperazine-1,4-dicarboxylate

To a solution of (R)-dibenzyl 2-(dimethylcarbamoyl)piperazine-1,4-dicarboxylate (28 g, 65.81 mmol, 1.0 equiv) in THF (300 mL) at 0° C. was added BH₃·Me₂S (10 M, 13.16 mL, 2.0 equiv). The reaction mixture was then stirred at 80° C. for 3 h. The reaction mixture was cooled to room temperature and then MeOH (50 mL) was added. After stirring for an additional 1 h the mixture was concentrated under reduced pressure. Purification by silica gel chromatography (50→100% EtOAc/petroleum ether) afforded the desired product (18 g, 66.5% yield) as a yellow oil.

### Step 3: Synthesis of (R)—N,N-dimethyl-1-(piperazin-2-yl)methanamine

To a solution of (S)-dibenzyl 2-((dimethylamino)methyl)piperazine-1,4-dicarboxylate (18 g, 43.74 mmol, 1.0 equiv) in EtOAc (200 mL) was added Pd/C (1.5 g, 10 wt. %). The suspension was degassed under reduced pressure and purged with H₂ three times. The suspension was stirred under H₂ (30 psi) at 30° C. for 5 h. The reaction mixture was then filtered through celite and the filtrate was concentrated under reduced pressure to afford the desired product (6 g, 95.8% yield) as a yellow solid.

### Step 4: Synthesis of tert-butyl N-tert-butoxycarbonyl-N-((2-((3S)-3-((dimethylamino)methyl)piperazin-1-yl)pyrimidin-5-yl)methyl)carbamate

To a solution of (R)—N,N-dimethyl-1-(piperazin-2-yl)methanamine (2.8 g, 19.55 mmol, 1.0 equiv) in MeCN (40 mL) was added tert-butyl N-tert-butoxycarbonyl-N-((2-chloropyrimidin-5-yl)methyl)carbamate (6.72 g, 19.55 mmol, 1.0 equiv) and K₂CO₃ (5.40 g, 39.10 mmol, 2.0 equiv). The mixture was stirred at 80° C. for 24 h. The mixture was then cooled to room temperature, filtered, and the filter cake washed with EtOAc (3×10 mL). The filtrate was then concentrated under reduced pressure. Purification by silica gel chromatography (0→100% MeOH/EtOAc) afforded the desired product (5.3 g, 57.8% yield) as a yellow oil. LCMS (ESI) m/z: [M+H] calcd for C₂₂H₃₈N₆O₄: 451.31; found 451.2.

### Building Block R. 2-[(2S)-4-[5-({[(tert-butoxy)carbonyl]amino}methyl)pyrimidin-2-yl]-2-[(dimethylamino)methyl]piperazin-1-yl]pyrimidine-5-carboxylic acid

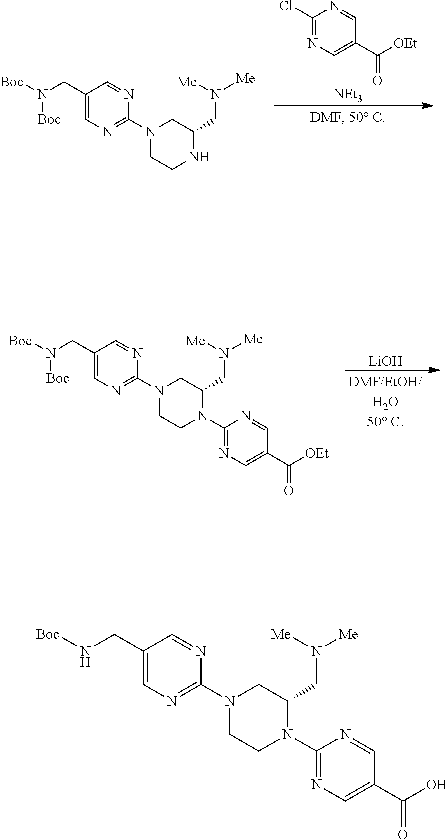 [SMILES: CN(C)C[C@H]1CN(c2ncc(CN(C(=O)OC(C)(C)C)C(=O)OC(C)(C)C)cn2)CCN1 ; CCOC(=O)c1cnc(Cl)nc1 ; CCOC(=O)c1cnc(N2CCN(c3ncc(CN(C(=O)OC(C)(C)C)C(=O)OC(C)(C)C)cn3)C[C@@H]2CN(C)C)nc1 ; "LiOH" ; "NEt3" ; CN(C)C[C@H]1CN(c2ncc(CNC(=O)OC(C)(C)C)cn2)CCN1c1ncc(C(=O)O)cn1]

### Step 1: Synthesis of (S)-ethyl 2-(4-(5-(((bi-tert-butoxycarbonyl)amino)methyl) pyrimidin-2-yl)-2-((dimethylamino)methyl)piperazin-1-yl)pyrimidine-5-carboxylate

To a solution of (S)-tert-butyl-N-tert-butoxycarbonyl ((2-(3-((dimethylamino)methyl) piperazin-1-yl)pyrimidin-5-yl)methyl)carbamate (3.26 g, 7.24 mmol, 1.0 equiv) in DMF (30 mL) was added Et₃N (3.02 mL, 21.71 mmol, 3.0 equiv) and ethyl 2-chloropyrimidine-5-carboxylate (1.47 g, 7.86 mmol, 1.1 equiv). The mixture was stirred at 50° C. for 3 h and then concentrated under reduced pressure to afford the desired product (4.35 g, crude) as a solution in DMF (30 mL), which was used directly in the next step. LCMS (ESI) m/z: [M+H] calcd for C₂₉H₄₄N₈O₆: 601.35; found 601.5.

### Step 2: Synthesis of (S)-2-(4-(5-(((tert-butoxycarbonyl)amino)methyl)-pyrimidin-2-yl)-2-((dimethylamino)methyl)piperazin-1-yl)pyrimidine-5-carboxylic acid

To a solution of (S)-ethyl 2-(4-(5-(((bi-tert-butoxycarbonyl)amino)methyl)-pyrimidin-2-yl)-2-((dimethylamino)methyl)piperazin-1-yl)pyrimidine-5-carboxylate (4.35 g, 7.24 mmol, 1.0 equiv) in DMF (30 mL) was added DMF (50 mL), EtOH (30 mL), and H₂O (30 mL). To the solution was then added LiOH·H₂O (3 g, 71.50 mmol, 9.9 equiv) at 50° C. The reaction was stirred at 50° C. for 36 h. The mixture was then cooled to room temperature, neutralized with 0.5 N HCl, and concentrated under reduced pressure. Purification by reverse phase chromatography (2→30% MeCN/H₂O) afforded the desired product (1.15 g, 34% yield) as a white solid. LCMS (ESI) m/z: [M+H] calcd for C₂₂H₃₂N₈O₄: 473.26; found 473.3.

### Building Block S. tert-butyl N-[(tert-butoxy)carbonyl]-N-({2-[(3R)-3-[(dimethylamino)methyl]piperazin-1-yl]pyrimidin-5-yl}methyl)carbamate

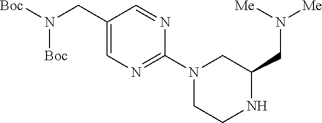 [SMILES: CN(C)C[C@@H]1CN(c2ncc(CN(C(=O)OC(C)(C)C)C(=O)OC(C)(C)C)cn2)CCN1]

This building block is prepared by a process similar to that for Building block I by utilizing dimethyl({[(2S)-piperazin-2-yl]methyl})amine.

### Building Block T. 2-[(2R)-4-[5-({[(tert-butoxy)carbonyl]amino}methyl)pyrimidin-2-yl]-2-[(dimethylamino)methyl]piperazin-1-yl]pyrimidine-5-carboxylicacid

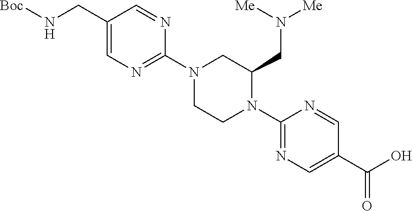 [SMILES: CN(C)C[C@@H]1CN(c2ncc(CNC(=O)OC(C)(C)C)cn2)CCN1c1ncc(C(=O)O)cn1]

This building block is prepared from Building block S by a process similar to that for Building block J.

### Building Block U. tert-butyl 2-[(3S)-3-[(dimethylamino)methyl]piperazin-1-yl]-5H,6H,7H,8H-pyrido[4,3-d]pyrimidine-6-carboxylate

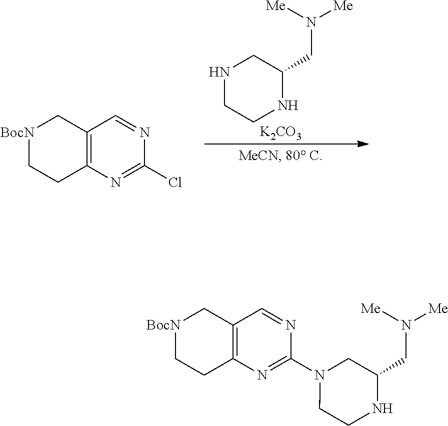

To a solution of tert-butyl 2-chloro-7,8-dihydropyrido[4,3-d]pyrimidine-6(5H)-carboxylate (4.80 g, 17.80 mmol, 1.4 equiv) in MeCN (45 mL) was added K₂CO₃ (10.42 g, 75.40 mmol, 3.0 equiv) and (R)—N,N-dimethyl-1-(piperazin-2-yl)methanamine (3.6 g, 25.13 mmol, 1.0 equiv). The mixture was stirred at 80° C. for 8 h. The mixture was then cooled to room temperature, filtered, and the filter cake was washed with EtOAc (50 mL). To the organic phase was added H₂O (50 mL) and the aqueous phase was extracted with EtOAc (3×50 mL). The combined organic phases were washed with brine (5 mL), dried, filtered and concentrated under reduced pressure. Purification by silica gel chromatography (8→67% EtOAc/petroleum ether) afforded the desired product (6.5 g, 63.5% yield) as a yellow oil.

### Building Block V. 2-[(2S)-4-{6-[(tert-butoxy)carbonyl]-5H,6H,7H,8H-pyrido[4,3-d]pyrimidin-2-yl}-2-[(dimethylamino)methyl]piperazin-1-yl]pyrimidine-5-carboxylic acid

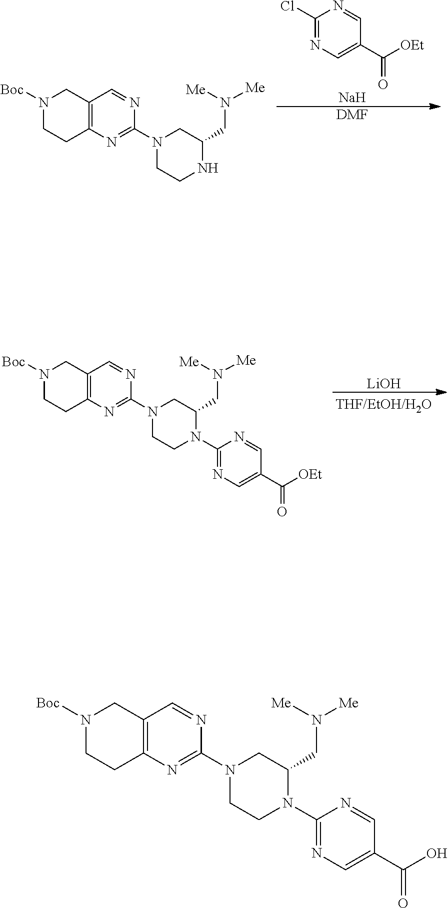

### Step 1: Synthesis of (S)-tert-butyl 2-(3-((dimethylamino)methyl)-4-(5-(ethoxycarbonyl)pyrimidin-2-yl)piperazin-1-yl)-7,8-dihydropyrido[4,3-d]pyrimidine-6(5H)-carboxylate

To a solution of (S)-tert-butyl 2-(3-((dimethylamino)methyl)piperazin-1-yl)-7,8-dihydropyrido[4,3-d]pyrimidine-6(5H)-carboxylate (3 g, 7.97 mmol, 1.0 equiv) in DMF (70 mL) at 0° C. was added NaH (382.44 mg, 9.56 mmol, 60 wt. %, 1.2 equiv). The suspension was stirred at 0° C. for 0.5 h, then ethyl 2-chloropyrimidine-5-carboxylate (1.49 g, 7.97 mmol, 1 equiv) in DMF (50 mL) was added, dropwise. The mixture was warmed to room temperature and stirred for 5 h. The mixture was then cooled to 0° C. and poured into H₂O (360 mL). The suspension was filtered, and the filter cake washed with H₂O (30 mL) and dried under reduced pressure. Purification by silica gel chromatography (6%→33% EtOAc/petroleum ether) afforded the desired product (1.8 g, 39.6% yield) as a brown oil.

### Step 2: Synthesis of (S)-2-(4-(6-(tert-butoxycarbonyl)-5,6,7,8-tetrahydropyrido[4,3-d]pyrimidin-2-yl)-2-((dimethylamino)methyl)piperazin-1-yl)pyrimidine-5-carboxylic acid

To a solution of (S)-tert-butyl 2-(3-((dimethylamino)methyl)-4-(5-(ethoxycarbonyl)pyrimidin-2-yl)piperazin-1-yl)-7,8-dihydropyrido[4,3-d]pyrimidine-6(5H)-carboxylate (1.1 g, 2.09 mmol, 1.0 equiv) in THF (5 mL), EtOH (2.5 mL), and H₂O (2.5 mL) was added LiOH·H₂O (175.30 mg, 4.18 mmol, 2.0 equiv). The mixture was stirred at room temperature for 2 h, at which point the pH was adjusted to 7 by the addition of 1 N HCl at 0° C. The mixture was concentrated under reduced pressure to remove THF and MeOH. The resulting suspension was filtered, and the filter cake was washed with H₂O (5 mL) and dried under reduced pressure to afford the desired product (680 mg, 65.3% yield) as a white solid. LCMS (ESI) m/z: [M+H] calcd for C₂₄H₃₄N₈O₄: 499.28; found 499.2.

### Building Block W. tert-butyl 2-[(3R)-3-[(dimethylamino)methyl]piperazin-1-yl]-5H,6H,7H,8H-pyrido[4,3-d]pyrimidine-6-carboxylate

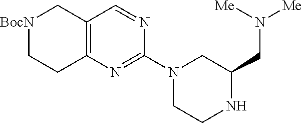 [SMILES: CN(C)C[C@@H]1CN(c2ncc3c(n2)CCN(C(=O)OC(C)(C)C)C3)CCN1]

This building block is prepared by a process similar to that for Building block I by utilizing tert-butyl 2-chloro-7,8-dihydropyrido[4,3-d]pyrimidine-6(5H)-carboxylate and dimethyl({[(2S)-piperazin-2-yl]methyl})amine.

### Building Block X. 2-[(2R)-4-{6-[(tert-butoxy)carbonyl]-5H,6H,7H,8H-pyrido[4,3-d]pyrimidin-2-yl}-2-[(dimethylamino)methyl]piperazin-1-yl]pyrimidine-5-carboxylic acid

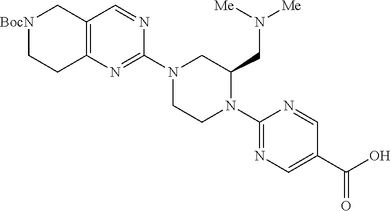 [SMILES: CN(C)C[C@@H]1CN(c2ncc3c(n2)CCN(C(=O)OC(C)(C)C)C3)CCN1c1ncc(C(=O)O)cn1]

This building block is prepared from Building block W by a process similar to that for Building block J.

### Building Block Y. tert-butyl (2R)-4-{6-[(tert-butoxy)carbonyl]-5H,6H,7H,8H-pyrido[4,3-d]pyrimidin-2-yl}piperazine-2-carboxylate

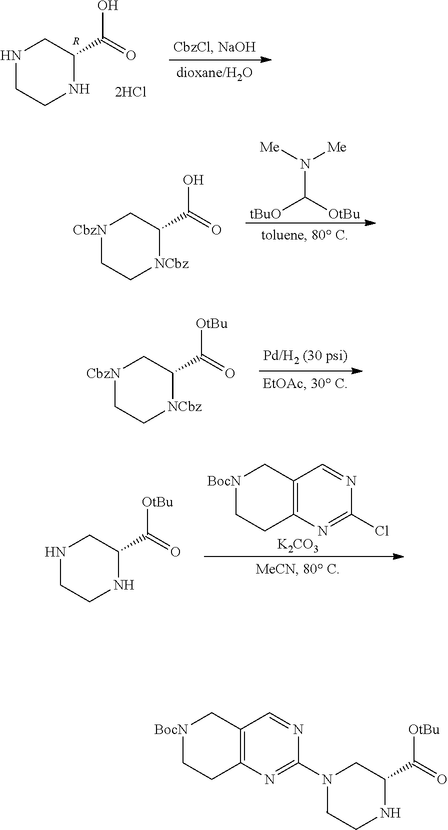

### Step 1: Synthesis of (R)-1,4-bis((benzyloxy)carbonyl)piperazine-2-carboxylic acid

To a solution of (R)-piperazine-2-carboxylic acid (70 g, 344.71 mmol, 1.0 equiv, 2HCl) in dioxane (1120 mL) and H₂O (700 mL) was added 50% aq. NaOH until the solution was pH=11. Benzyl chloroformate (156.82 mL, 1.10 mol, 3.2 equiv) was added and the reaction mixture was stirred at room temperature for 12 h. To the solution was then added H₂O (1200 mL) and the aqueous layer was washed with MTBE (3×800 mL). The aqueous layer was adjusted to pH=2 with concentrated HCl (12N) and extracted with EtOAc (2×1000 mL). The combined organic extracts were dried, filtered and the filtrate was concentrated under reduced pressure to afford the desired product (137 g, 99.8% yield) as a yellow solid. LCMS (ESI) m/z: [M+H] calcd for C₂₁H₂₂N₂O₆: 399.16; found 399.2.

### Step 2: Synthesis of (R)-1,4-dibenzyl 2-tert-butyl piperazine-1,2,4-tricarboxylate

To a solution of (R)-1,4-bis((benzyloxy)carbonyl)piperazine-2-carboxylic acid (50 g, 125.50 mmol, 1.0 equiv) in toluene (500 mL) at 80° C. was added 1,1-di-tert-butoxy-N,N-dimethylmethanamine (57.17 mL, 238.45 mmol, 1.9 equiv). The solution was stirred at 80° C. for 2 h, at which point the reaction mixture was cooled to room temperature and partitioned between EtOAc (300 mL) and H₂O (500 mL). The aqueous layer was extracted with EtOAc (2×500 mL) and the combined organic layers were dried, filtered and the filtrate was concentrated under reduced pressure. Purification by silica gel chromatography (0%→25% EtOAc/petroleum ether) afforded the desired product (35 g, 61.2% yield) as a white solid. LCMS (ESI) m/7: [M+Na] calcd for C₂₅H₃₀N₂O₆: 477.20; found 477.1.

### Step 3: Synthesis of (R)-tert-butyl piperazine-2-carboxylate

To a solution of (R)-1,4-dibenzyl 2-tert-butyl piperazine-1,2,4-tricarboxylate (35 g, 77.01 mmol, 1.0 equiv) in EtOAc (350 mL) was added Pd/C (10 g, 10 wt. %). The suspension was degassed under reduced pressure and purged with H₂ three times. The mixture was stirred under H₂ (30 psi) at 30° C. for 4 h. The reaction mixture was then filtered through celite, the residue was washed with MeOH (5×200 mL), and the filtrate was concentrated under reduced pressure to afford the desired product (14 g, 79.6% yield) as yellow oil. LCMS (ESI) m/z: [M+H] calcd for C₉H₁₈N₂O₂: 187.15; found 187.1.

### Step 4: Synthesis of (R)-tert-butyl 2-(3-(tert-butoxycarbonyl)piperazin-1-yl)-7,8-dihydropyrido[4,3-d]pyrimidine-6(5H)-carboxylate

To a solution of tert-butyl (2R)-piperazine-2-carboxylate (12 g, 64.43 mmol, 1.0 equiv) in MeCN (200 mL) was added K₂CO₃ (17.81 g, 128.86 mmol, 2.0 equiv) and tert-butyl 2-chloro-7,8-dihydropyrido[4,3-d]pyrimidine-6(5H)-carboxylate (17.38 g, 64.43 mmol, 1.0 equiv). The reaction mixture was heated to 80° C. and stirred for 12 h. The reaction mixture was then cooled to room temperature and filtered, the residue was washed with EtOAc (3×150 mL), and the filtrate was concentrated under reduced pressure. Purification by silica gel chromatography (0%→100% EtOAc/petroleum ether) afforded the desired product (19 g, 69.2% yield) as a yellow solid. LCMS (ESI) m/z: [M+H] calcd for C₂₁H₃₃N₅O₄: 420.26; found 420.2.

### Building Block Z. 4-amino-2-[(2R)-2-[(tert-butoxy)carbonyl]-4-{6-[(tert-butoxy)carbonyl]-5H,6H,7H,8H-pyrido[4,3-d]pyrimidin-2-yl}piperazin-1-yl]pyrimidine-5-carboxylic acid

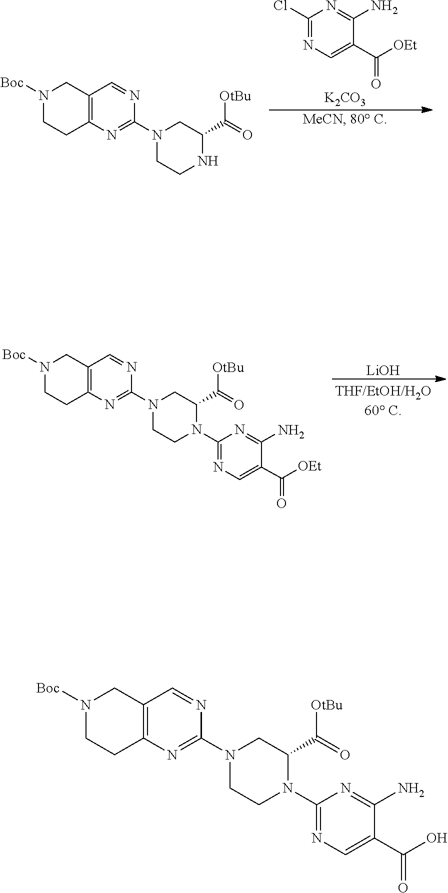

### Step 1: Synthesis of (R)-tert-butyl 2-(4-(4-amino-5-(ethoxycarbonyl)pyrimidin-2-yl)-3-(tert-butoxycarbonyl)piperazin-1-yl)-7,8-dihydropyrido[4,3-d]pyrimidine-6(5H)-carboxylate

To a stirred solution of (R)-tert-butyl 2-(3-(tert-butoxycarbonyl)piperazin-1-yl)-7,8-dihydropyrido[4,3-d]pyrimidine-6(5H)-carboxylate (12 g, 28.60 mmol, 1.0 equiv) in MeCN (150 mL) was added K₂CO₃ (7.91 g, 57.20 mmol, 2.0 equiv) and ethyl 4-amino-2-chloropyrimidine-5-carboxylate (6.92 g, 34.32 mmol, 1.2 equiv). The reaction mixture was stirred at 80° C. for 12 h, at which point the reaction mixture was filtered and the filtrate was concentrated under reduced pressure. Purification by silica gel chromatography (0%→17% EtOAc/petroleum ether) afforded the desired product (16 g, 91.6% yield) as a yellow solid. LCMS (ESI) m/z: [M+H] calcd for C₂₈H₄₀N₈O₆: 585.32; found 585.1.

### Step 2: Synthesis of (R)-4-amino-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

To two separate batches run in parallel each containing a solution of (R)-tert-butyl 2-(4-(4-amino-5-(ethoxycarbonyl)pyrimidin-2-yl)-3-(tert-butoxycarbonyl)piperazin-1-yl)-7,8-dihydropyrido[4,3-d]pyrimidine-6(5H)-carboxylate (7 g, 11.97 mmol, 1.0 equiv) in THF (70 mL), EtOH (35 mL) and H₂O (35 mL) was added LiOH·H₂O (2.01 g, 47.89 mmol, 4.0 equiv). The mixtures were stirred at 60° C. for 3 h, at which point the two reaction mixtures were combined, and were adjusted to pH=7 with 1 N HCl. The mixture was concentrated under reduced pressure to remove THF and EtOH, filtered, and the residue was dried under reduced pressure. The residue was stirred in MTBE (100 mL) for 10 min, filtered, and the residue was dried under reduced pressure to afford the desired product (8.02 g, 55.1% yield) as a white solid. LCMS (ESI) m/z: [M+H] calcd for C₂₆H₃₆N₈O₆:557.29; found 557.3.

### Building Block AA. tert-butyl (2S)-4-{6-[(tert-butoxy)carbonyl]-5H,6H,7H,8H-pyrido[4,3-d]pyrimidin-2-yl}piperazine-2-carboxylate

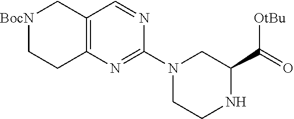 [SMILES: CC(C)(C)OC(=O)[C@@H]1CN(c2ncc3c(n2)CCN(C(=O)OC(C)(C)C)C3)CCN1]

This building block is prepared by a process similar to that for Building block I by utilizing tert-butyl 2-chloro-7,8-dihydropyrido[4,3-d]pyrimidine-6(5H)-carboxylate and tert-butyl (2S)-piperazine-2-carboxylate.

### Building Block AB. 4-amino-2-[(2S)-2-[(tert-butoxy)carbonyl]-4-{6-[(tert-butoxy)carbonyl]-5H,6H,7H,8H-pyrido[4,3-d]pyrimidin-2-yl}piperazin-1-yl]pyrimidine-5-carboxylic acid

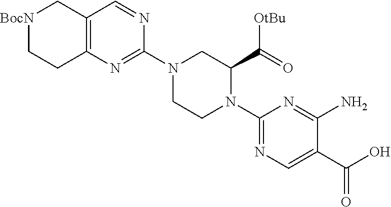 [SMILES: CC(C)(C)OC(=O)[C@@H]1CN(c2ncc3c(n2)CCN(C(=O)OC(C)(C)C)C3)CCN1c1ncc(C(=O)O)c(N)n1]

This building block is prepared from Building block AA by a process similar to that for Building block J by utilizing ethyl 4-amino-2-chloropyrimidine-5-carboxylate.

### Building Block AC. 4-amino-2-(4-{6-[(tert-butoxy)carbonyl]-5H,6H,7H,8H-pyrido[4,3-d]pyrimidin-2-yl}piperazin-1-yl)pyrimidine-5-carboxylic acid

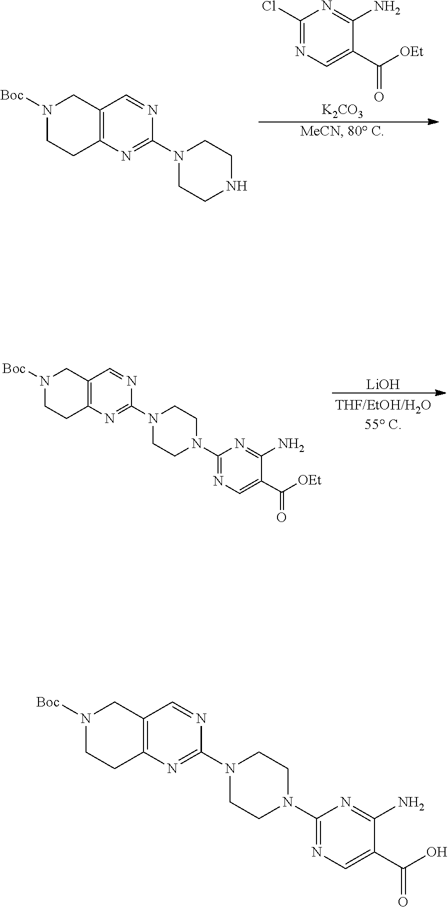

### Step 1: Synthesis of tert-butyl 2-(4-(4-amino-5-(ethoxycarbonyl)pyrimidin-2-yl)piperazin-1-yl)-7,8-dihydropyrido[4,3-d]pyrimidine-6(5H)-carboxylate

To a solution of tert-butyl 2-(piperazin-1-yl)-7,8-dihydropyrido[4,3-d]pyrimidine-6(5H)-carboxylate (8.3 g, 25.99 mmol, 1.0 equiv) and ethyl 4-amino-2-chloropyrimidine-5-carboxylate (5.24 g, 25.99 mmol, 1.0 equiv) in MeCN (100 mL) was added to K₂CO₃ (7.18 g, 51.97 mmol, 2.0 equiv). The reaction was stirred at 80° C. for 12 h. The reaction was then cooled to room temperature, DCM (100 mL) was added, and the reaction mixture was stirred for 30 min. The suspension was filtered, and the filter cake was washed with DCM (6×100 mL). The filtrate was concentrated under reduced pressure and the residue was triturated with EtOAc (30 mL), filtered and then the filter cake was dried under reduced pressure to afford the desired product (8.7 g, 65.9% yield) as light yellow solid.

### Step 2: Synthesis of 4-amino-2-(4-(6-(tert-butoxycarbonyl)-5,6,7,8-tetrahydropyrido[4,3-d]pyrimidin-2-yl)piperazin-1-yl)pyrimidine-5-carboxylic acid

To a solution of tert-butyl 2-(4-(4-amino-5-(ethoxycarbonyl)pyrimidin-2-yl)piperazin-1-yl)-7,8-dihydropyrido[4,3-d]pyrimidine-6(5H)-carboxylate (8.7 g, 17.95 mmol, 1.0 equiv) in THF (120 mL), EtOH (60 mL), and H₂O (60 mL) was added LiOH·H₂O (1.51 g, 35.91 mmol, 2.0 equiv). The mixture was stirred at 55° C. for 12 h. The reaction mixture was then concentrated under reduced pressure to remove EtOH and THF, and the reaction mixture was adjusted to pH=6 by the addition of 1 N HCl. The precipitate was filtered, and the filter cake was washed with H₂O (3×50 mL) and then dried under reduced pressure to afford the desired product (7.3 g, 89.1% yield) as light yellow solid. LCMS (ESI) m/z: [M+H] calcd for C₂₁H₂₈N₈O₄: 457.23; found 457.2.

### Building Block AD. 4-amino-2-{4-[5-({[(tert-butoxy)carbonyl]amino}methyl)pyrimidin-2-yl]piperazin-1-yl}pyrimidine-5-carboxylic acid

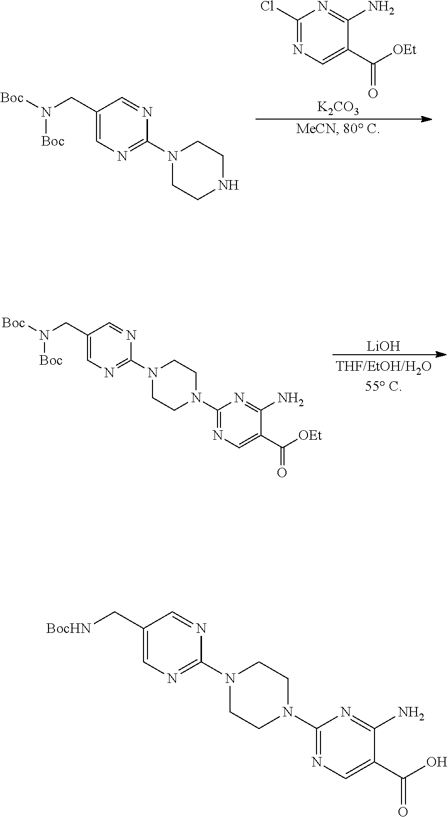

### Step 1: Synthesis of ethyl 4-amino-2-(4-(5-(((di-tert-butoxycarbonyl)amino)methyl)pyrimidin-2-yl)piperazin-1-yl)pyrimidine-5-carboxylate

To a solution of tert-butyl-N-tert-butoxycarbonyl-N-((2-(piperazin-1-yl)pyrimidin-5-yl)methyl)carbamate (8.3 g, 21.09 mmol, 1.0 equiv) in MeCN (100 mL) was added ethyl 4-amino-2-chloropyrimidine-5-carboxylate (4.04 g, 20.04 mmol, 0.95 equiv) and K₂CO₃ (8.75 g, 63.28 mmol, 3.0 equiv). The mixture was stirred at 80° C. for 3 h. The reaction was then cooled to room temperature. DCM (150 mL) was added, and the reaction mixture was stirred for 30 min. The suspension was filtered, the filter cake was washed with DCM (3×100 mL), and the filtrate was concentrated under reduced pressure. Purification by silica gel chromatography (0%→100% EtOAc/petroleum ether) afforded the desired product (8.35 g, 67% yield) as a white solid.

### Step 2: Synthesis of 4-amino-2-(4-(5-(((tert-butoxycarbonyl)amino)methyl)pyrimidin-2-yl)piperazin-1-yl)pyrimidine-5-carboxylic acid

To a solution of ethyl 4-amino-2-(4-(5-(((di-tert-butoxycarbonyl)amino)methyl)pyrimidin-2-yl)piperazin-1-yl)pyrimidine-5-carboxylate (8.3 g, 14.86 mmol, 1.0 equiv) in H₂O (70 mL), EtOH (36 mL) and THF (80 mL) was added LiOH·H₂O (2.49 g, 59.43 mmol, 4.0 equiv). The reaction mixture was stirred at 55° C. for 16 h. The mixture was then concentrated under reduced pressure to remove THF and EtOH. The mixture was diluted with H₂O (55 mL) and was adjusted to pH=6 by the addition of 1 N HCl. The mixture was filtered, and the filter cake was washed with H₂O (2×20 mL). The solid cake was dried under reduced pressure to afford the desired product (5.5 g, 84% yield) as a white solid. LCMS (ESI) m/z: [M+H] calcd for C₁₉H₂₆N₈O₄: 431.22; found 431.4.

### Building Block AE. 4-amino-2-[(2R)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

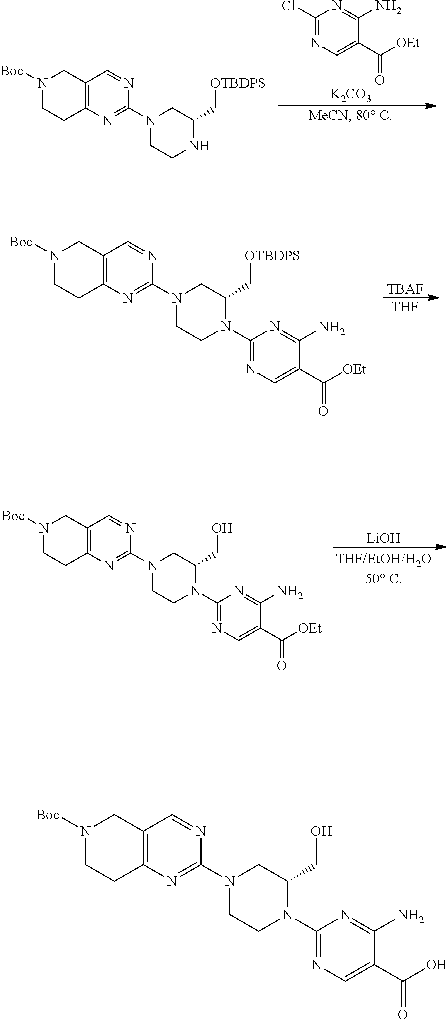

### Step 1: Synthesis of (R)-tert-butyl 2-(4-(4-amino-5-(ethoxycarbonyl)pyrimidin-2-yl)-3-(((tert-butyldiphenylsilyl)oxy)methyl)piperazin-1-yl)-7,8-dihydropyrido[4,3-d]pyrimidine-6(5H)-carboxylate

To a solution of (R)-tert-butyl 2-(3-(((tert-butyldiphenylsilyl)oxy)methyl) piperazin-1-yl)-7,8-dihydropyrido[4,3-d]pyrimidine-6(5H)-carboxylate (17.2 g, 29.26 mmol, 1.0 equiv) in MeCN (200 mL) was added K₂CO₃ (12.13 g, 87.78 mmol, 3.0 equiv) and ethyl 4-amino-2-chloropyrimidine-5-carboxylate (6.37 g, 31.60 mmol, 1.08 equiv). The mixture was stirred at 80° C. for 18 h. The reaction mixture was then cooled to room temperature, filtered and the filtrate was concentrated under reduced pressure. Purification by silica gel chromatography (0%→33% EtOAc/petroleum ether) afforded the desired product (20.3 g, 90.6% yield) as a white solid. LCMS (ESI) m/z: [M+H] calcd for C₄₀H₅₂N₈O₅Si: 753.39; found 753.4.

### Step 2: Synthesis of (R)-4-amino-2-(4-(6-(tert-butoxycarbonyl)-5,6,7,8-tetrahydropyrido[4,3-d]pyrimidin-2-yl)-2-(hydroxymethyl)piperazin-1-yl)pyrimidine-5-carboxylic acid

To a solution of (R)-tert-butyl 2-(4-(4-amino-5-(ethoxycarbonyl)pyrimidin-2-yl)-3-(((tert-butyldiphenylsilyl)oxy)methyl)piperazin-1-yl)-7,8-dihydropyrido[4,3-d]pyrimidine-6(5H)-carboxylate (20.3 g, 26.96 mmol, 1.0 equiv) in THF (200 mL) was added TBAF (1.0 M, 50.75 mL, 1.9 equiv). The reaction mixture was stirred at room temperature for 5 h. The mixture was then poured into H₂O (200 mL) and the aqueous phase was extracted with EtOAc (2×150 mL). The combined organic phases were washed with brine (2×100 mL), dried, filtered and concentrated under reduced pressure. Purification by silica gel chromatography (0%→20% EtOAc/petroleum ether) afforded the desired product (12 g, 85.7% yield) as a white solid. LCMS (ESI) m/z: [M+H] calcd for C₂₄H₃₄N₈O₅: 515.28; found 515.4.

### Step 3: Synthesis of (R)-4-amino-2-(4-(6-(tert-butoxycarbonyl)-5,6,7,8-tetrahydropyrido[4,3-d]pyrimidin-2-yl)-2-(hydroxymethyl)piperazin-1-yl)pyrimidine-5-carboxylic acid

To a solution of (R)-4-amino-2-(4-(6-(tert-butoxycarbonyl)-5,6,7,8-tetrahydropyrido[4,3-d]pyrimidin-2-yl)-2-(hydroxymethyl)piperazin-1-yl)pyrimidine-5-carboxylic acid (12 g, 23.32 mmol, 1.0 equiv) in THF (100 mL), EtOH (30 mL), and H₂O (30 mL) was added LiOH·H₂O (5.87 g, 139.92 mmol, 6.0 equiv). The mixture was stirred at 50° C. for 22 h. The mixture was then concentrated under reduced pressure to remove THF and EtOH. The aqueous phase was neutralized with 1 N HCl and the resulting precipitate was filtered. The filter cake was washed with H₂O (50 mL) and dried under reduced pressure. The filtrate was extracted with DCM (8×60 mL) and the combined organic phases were washed with brine (2×50 mL), dried, filtered, and concentrated under reduced pressure. The resulting residue was combined with the initial filter cake and the solid was dissolved in DCM (150 mL) and concentrated under reduced pressure to afford the desired product (9.76 g, 85.2% yield) as a white solid. LCMS (ESI) m/z: [M+H] calcd for C₂₂H₃N₈O₅: 487.24; found 487.2.

### Building Block AF. 4-amino-2-[(2S)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

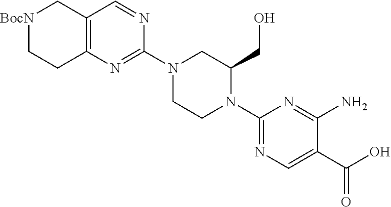 [SMILES: CC(C)(C)OC(=O)N1CCc2nc(N3CCN(c4ncc(C(=O)O)c(N)n4)[C@H](CO)C3)ncc2C1]

This building block is prepared from Building block O by a process similar to that for Building block J by utilizing ethyl 4-amino-2-chloropyrimidine-5-carboxylate.

### Building Block AG. 2-((2-(4-(5-((di-(tert-butoxycarbonyl)amino)methyl)pyrimidin-2-yl)piperazin-1-yl)-2-oxoethyl)(methyl)amino)acetic acid

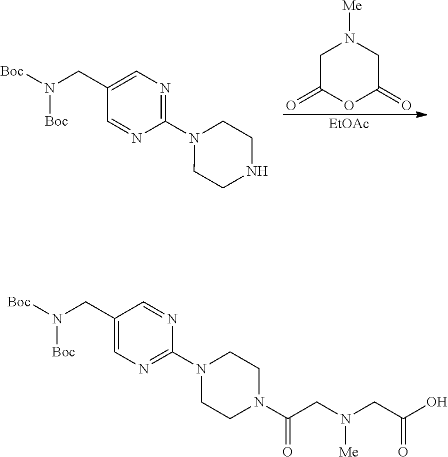 [SMILES: "EtOAc" ; CC(C)(C)OC(=O)N(Cc1cnc(N2CCNCC2)nc1)C(=O)OC(C)(C)C ; CN1CC(=O)OC(=O)C1 ; CN(CC(=O)O)CC(=O)N1CCN(c2ncc(CN(C(=O)OC(C)(C)C)C(=O)OC(C)(C)C)cn2)CC1]

To a solution of tert-butyl N-tert-butoxycarbonyl-N-((2-piperazin-1-ylpyrimidin-5-yl)methyl)carbamate (4.88 g, 12.39 mmol, 1.0 equiv) in EtOAc (40 mL) was added 4-methylmorpholine-2,6-dione (1.6 g, 12.39 mmol, 1.0 equiv). The reaction was stirred at room temperature for 2 h then reaction mixture was concentrated under reduced pressure to give the crude product. The residue was triturated with EtOAc (15 mL) and filtered to give the product (5.65 g, 87.2% yield) as a white solid. LCMS (ESI) m/z: [M+H] calcd for C₂₄H₃₉N₆O₇: 523.28; found 523.3.

### Building Block AH. tert-butyl N-tert-butoxycarbonyl-N-((2-(4-(3-(2-piperazin-1-ylethoxy)propanoyl)piperazin-1-yl)pyrimidin-5-yl)methyl)carbamate

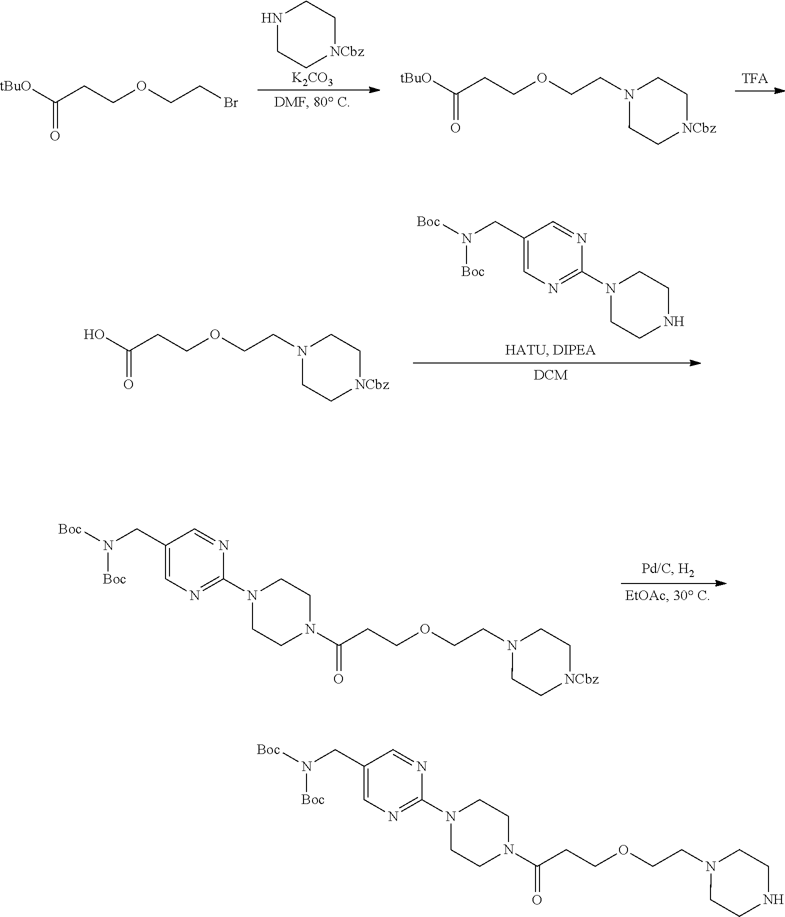

### Step 1: Synthesis of benzyl 4-(2-(3-(tert-butoxy)-3-oxopropoxy)ethyl)piperazine-1-carboxylate

To a solution of tert-butyl 3-(2-bromoethoxy)propanoate (35 g, 138.27 mmol, 1.0 equiv) and benzyl piperazine-1-carboxylate (31.14 mL, 138.27 mmol, 1.0 equiv, HCl) in MeCN (420 mL) was added K₂CO₃ (57.33 g, 414.80 mmol, 3.0 equiv). The reaction was stirred at 80° C. for 20 h. The reaction mixture was cooled to room temperature and the suspension was filtered. The filter cake was washed with EtOAc (3×50 mL) and the combined filtrates were concentrated under reduced pressure to give crude product. The residue was purified by silica gel chromatography (5/1 to 0/1 petroleum ether/EtOAc) to give the product (46 g, 84.8% yield) as a yellow oil.

### Step 2: Synthesis of 3-(2-(4-((benzyloxy)carbonyl)piperazin-1-yl)ethoxy)propanoic acid

A solution of benzyl 4-(2-(3-(tert-butoxy)-3-oxopropoxy)ethyl)piperazine-1-carboxylate (21 g, 53.50 mmol, 1.0 equiv) in TFA (160 mL) was stirred at room temperature for 2 h and then concentrated under reduced pressure. The residue was purified by silica gel chromatography (1/0 to 4/1 EtOAc/MeOH) to give the product (20.4 g, 84.7% yield) as a yellow oil. LCMS (ESI) m/z: [M+H] calcd for C₁₇H₂₄N₂O₅: 337.18; found 337.1.

### Step 3: Synthesis of benzyl 4-(2-(3-(4-(5-(((di-tert-butoxycarbonyl)amino)methyl)pyrimidin-2-yl)piperazin-1-yl)-3-oxopropoxy)ethyl)piperazine-1-carboxylate

To a solution of 3-(2-(4-((benzyloxy)carbonyl)piperazin-1-yl)ethoxy)propanoic acid (20.2 g, 44.85 mmol, 1.0 equiv. TFA) in DCM (500 mL) was added HATU (25.58 g, 67.27 mmol, 1.5 equiv) and DIPEA (17.39 g, 134.55 mmol, 23.44 mL, 3.0 equiv). The reaction was stirred at room temperature for 30 min, and then tert-butyl N-tert-butoxycarbonyl-N-((2-piperazin-1-ylpyrimidin-5-yl)methyl)carbamate (14.12 g, 35.88 mmol, 0.8 equiv) was added. The reaction mixture was stirred at for 2 h and then quenched with sat. NH₄Cl (500 mL). The aqueous phase was extracted with DCM (3×300 mL) and the combined organic phase was washed with brine (30 mL), dried with anhydrous Na₂SO₄, filtered and concentrated under reduced pressure to give crude product. The residue was purified by silica gel chromatography (0/1 petroleum ether/EtOAc to 10/1 DCM/MeOH) to give the product (29 g, 90.8% yield) as a yellow oil. LCMS (ESI) m/z: [M+H] calcd for C₃₆H₃N₇O₈: 712.41; found 712.4.

### Step 4: Synthesis of tert-butyl N-tert-butoxycarbonyl-N-((2-(4-(3-(2-piperazin-1-ylethoxy)propanoyl)piperazin-1-yl)pyrimidin-5-yl)methyl)carbamate

To a solution of 4-(2-(3-(4-(5-(((di-tert-butoxycarbonyl)amino)methyl)pyrimidin-2-yl)piperazin-1-yl)-3-oxopropoxy)ethyl)piperazine-1-carboxylate (5 g, 7.02 mmol, 1.0 equiv) in EtOAc (150 mL) was added Pd/C (2 g, 10 wt. %). The suspension was degassed and purged with H₂ and then stirred under H₂ (30 psi) at 30° C. for 3 h. The suspension was then cooled to room temperature and filtered through Celite. The filter cake was washed with MeOH (15×100 mL) and the combined filtrates were concentrated under reduced pressure to give the product (12 g, 89.9% yield) as a light yellow oil. LCMS (ESI) m/z: [M+H] calcd for C₂₈H₄₇N₇O₆: 578.37; found 578.5.

### Building Block AI. ethyl 2-(piperazin-1-yl)pyrimidine-5-carboxylate

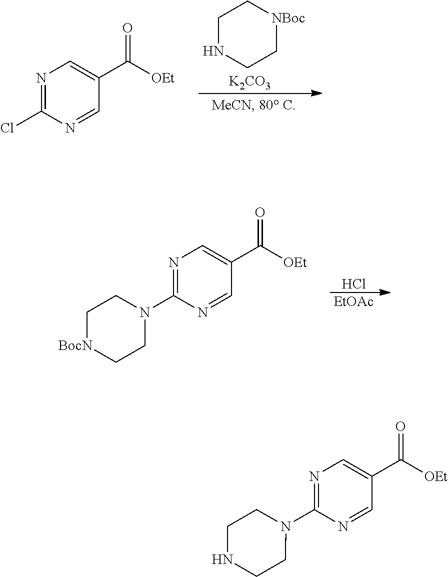

### Step 1: Synthesis of ethyl 2-(4-(tert-butoxycarbonyl)piperazin-1-yl)pyrimidine-5-carboxylate

To a solution of tert-butyl piperazine-1-carboxylate (11.94 g, 53.59 mmol, 1.0 equiv, HCl) and ethyl 2-chloropyrimidine-5-carboxylate (10 g, 53.59 mmol, 1.0 equiv) in MeCN (100 mL) was added K₂CO₃ (7.41 g, 53.59 mmol, 1.0 equiv). The mixture was stirred at 80° C. for 17 h and then poured into H₂O (200 mL). The mixture was filtered and the filter cake was washed with H₂O (80 mL) and dried under reduced pressure to give the product (15.76 g, 82% yield) as a white solid.

### Step 2: Synthesis of ethyl 2-(piperazin-1-yl)pyrimidine-5-carboxylate

To a solution of ethyl 2-(4-(tert-butoxycarbonyl)piperazin-1-yl)pyrimidine-5-carboxylate (15.7 g, 46.67 mmol, 1.0 equiv) in EtOAc (150 mL) was added HCl/EtOAc (150 mL) at 0° C. The resulting mixture was stirred at room temperature for 9 h. The reaction mixture was filtered and the filter cake was washed with EtOAc (100 mL). The solid was dried under reduced pressure to give the product (12.55 g, 96% yield, HCl) as a white solid. LCMS (ESI) m/z: [M+H] calcd for C₁₁H₁₆N₄O₂: 237.14; found 237.3.

### Building Block AJ. 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylic acid

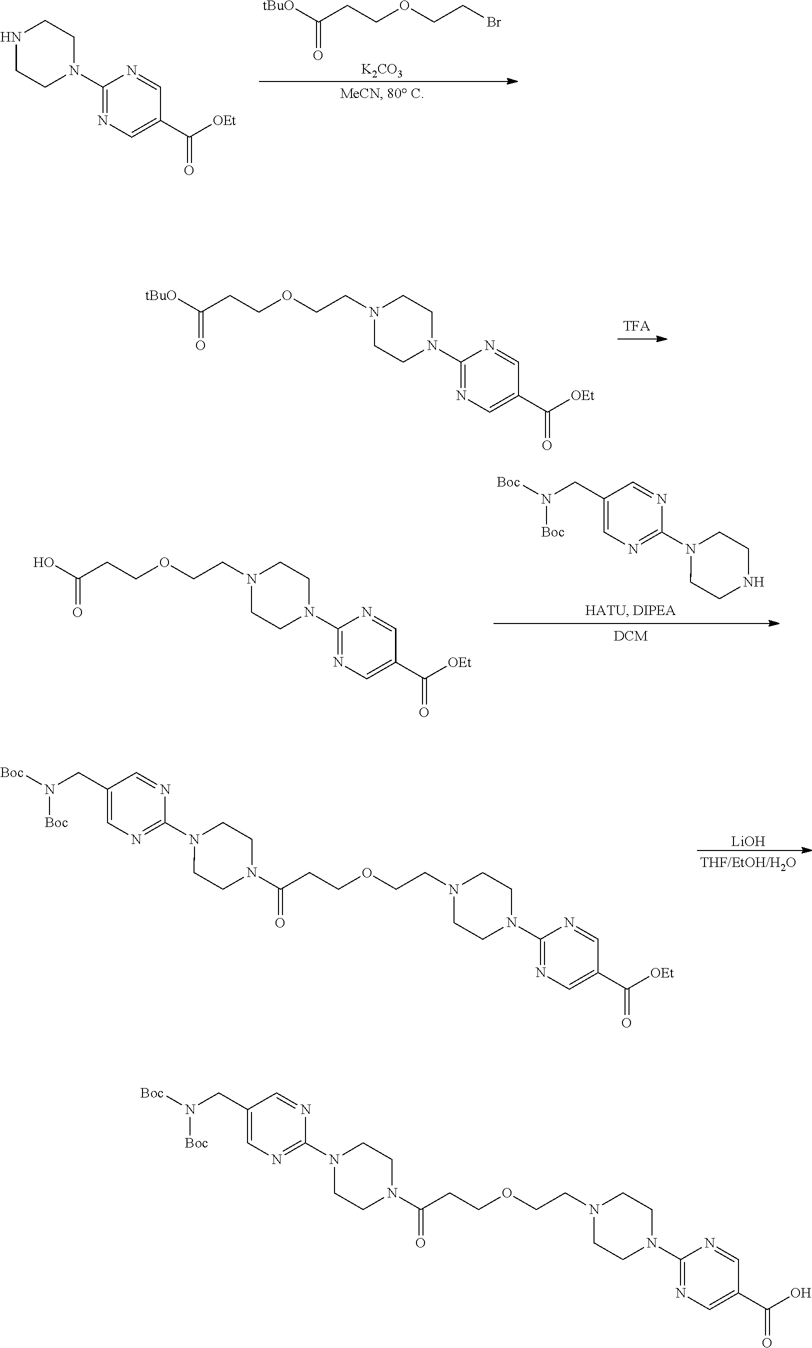

### Step 1: Synthesis of ethyl 2-(4-(2-(3-(tert-butoxy)-3-oxopropoxy)ethyl)piperazin-1-yl)pyrimidine-5-carboxylate

To a solution of ethyl 2-piperazin-1-ylpyrimidine-5-carboxylate (17.92 g, 75.85 mmol, 1.2 equiv) and tert-butyl 3-(2-bromoethoxy)propanoate (16 g, 63.21 mmol, 1.0 equiv) in MeCN (200 mL) was added K₂CO₃ (17.47 g, 126.42 mmol, 2.0 equiv). The reaction was stirred at 80° C. for 12 h and then the reaction mixture was filtered, and the filtrate was concentrated under reduced pressure. The crude product was suspended in petroleum ether (200 mL) and stirred for 20 min at 0° C. and then filtered. The solid was dried under reduced pressure to give the product (19.4 g, 75.1% yield) as a yellow solid.

### Step 2: Synthesis of 3-(2-(4-(5-(ethoxycarbonyl)pyrimidin-2-yl)piperazin-1-yl)ethoxy)propanoic acid

A solution of ethyl 2-(4-(2-(3-(tert-butoxy)-3-oxopropoxy)ethyl)piperazin-1-yl)pyrimidine-5-carboxylate (19.4 g, 47.49 mmol, 1.0 equiv) in TFA (200 mL) was stirred at room temperature for 30 min. The reaction mixture was then concentrated under reduced pressure and the residue was purified by silica gel chromatography (50/1 to 1/1 EtOAc/MeOH) to give the product (18 g, 81.3% yield) as a yellow oil.

### Step 3: Synthesis of ethyl 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylate

To a solution of 3-(2-(4-(5-(ethoxycarbonyl)pyrimidin-2-yl)piperazin-1-yl)ethoxy)propanoic acid (13 g, 27.87 mmol, 1.0 equiv) in DCM (200 mL) was added HATU (15.90 g, 41.81 mmol, 1.5 equiv) and DIPEA (19.42 mL, 111.49 mmol, 4.0 equiv). The reaction was then stirred at room temperature for 30 min and then tert-butyl N-tert-butoxycarbonyl-N-[(2-piperazin-1-ylpyrimidin-5-yl)methyl]carbamate (10.97 g, 27.87 mmol, 1.0 equiv) was added. The mixture was stirred at for 2 h and then poured into a sat. NH₄Cl solution (200 mL). The aqueous phase was extracted with DCM (2×200 mL) and the combined organic phase was washed with brine (2×20 mL), dried with anhydrous Na₂SO₄, filtered and concentrated under reduced pressure. The residue was purified by silica gel chromatography (100/1 to 9/1 EtOAc/MeOH) to give the product (17 g, 79.0% yield) as a yellow oil.

### Step 4: Synthesis of 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylic acid

To a solution of ethyl 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylate (11 g, 15.11 mmol, 1.0 equiv) in THF (40 mL), EtOH (10 mL), and H₂O (20 mL) was added LiOH·H₂O (1.27 g, 30.23 mmol, 2.0 equiv). The mixture was then stirred at 35° C. for 1.5 h. The reaction mixture was extracted with EtOAc (30 mL) and the aqueous phase was adjusted to pH=7 by addition of HCl (1 N). The mixture was then concentrated under reduced pressure. The crude product was purified by reversed-phase chromatography (20/1 to 3/1 H₂O/MeCN) to give the product (6.1 g, 67.3% yield) as a white solid. LCMS (ESI) m/z: [M+H] calcd for C₃₃H₄₉N₉O₈: 700.38; found 700.4.

### Building Block AK. 2-(4-(2-(3-(4-(5-(((tert-butoxycarbonyl)amino)methyl)pyrimidin-2-yl)piperazin-1-yl)-3-oxopropoxy)ethyl)piperazin-1-yl)pyrimidine-5-carboxylic acid

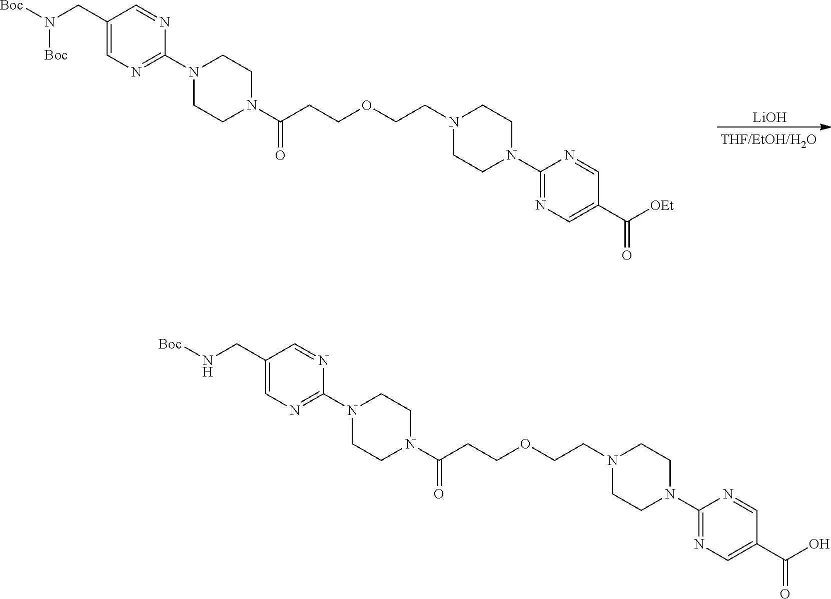 [SMILES: CCOC(=O)c1cnc(N2CCN(CCOCCC(=O)N3CCN(c4ncc(CN(C(=O)OC(C)(C)C)C(=O)OC(C)(C)C)cn4)CC3)CC2)nc1 ; "LiOH" ; CC(C)(C)OC(=O)NCc1cnc(N2CCN(C(=O)CCOCCN3CCN(c4ncc(C(=O)O)cn4)CC3)CC2)nc1]

A solution of ethyl 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylate (5.4 g, 7.42 mmol, 1.0 equiv) in THF (40 mL), EtOH (10 mL), and H₂O (10 mL) was added LiOH·H₂O (933.92 mg, 22.26 mmol, 3.0 equiv). The mixture was then stirred at 30° C. for 12 h. The reaction mixture was then extracted with EtOAc (2×50 mL) and the aqueous phase was adjusted to pH=7 by addition of HCl (1 N). The solution was then concentrated under reduced pressure. The crude product was purified by reversed-phase chromatography (20/1 to 3/1 H₂O/MeCN) to give the product (1.01 g, 22.5% yield) as a white solid. LCMS (ESI) m/z: [M+H] calcd for C₂₈H₄₁N₉O₆: 600.33; found 600.2.

### Building Block AL. 4-{4-[2-(3-{4-[5-({[(tert-butoxy)carbonyl]amino}methyl)pyrimidin-2-yl]piperazin-1-yl}-3-oxopropoxy)ethyl]piperazin-1-yl}-4-oxobutanoic acid

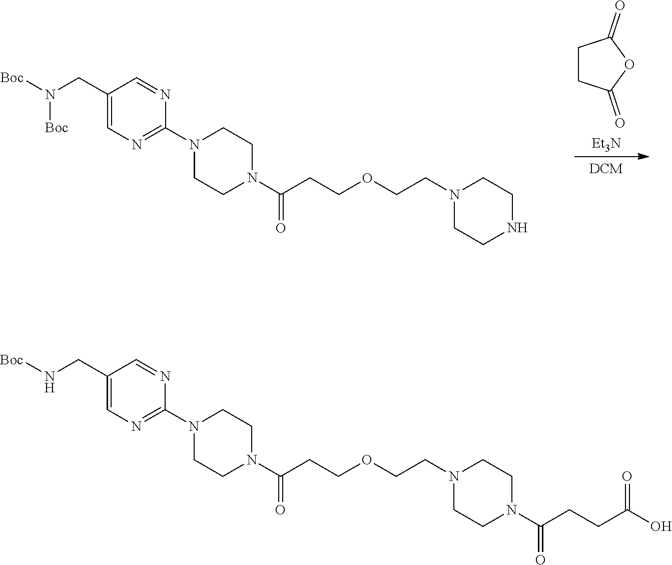 [SMILES: "Et3N" ; CC(C)(C)OC(=O)N(Cc1cnc(N2CCN(C(=O)CCOCCN3CCNCC3)CC2)nc1)C(=O)OC(C)(C)C ; O=C1CCC(=O)O1 ; CC(C)(C)OC(=O)NCc1cnc(N2CCN(C(=O)CCOCCN3CCN(C(=O)CCC(=O)O)CC3)CC2)nc1]

To a solution of tert-butyl N-tert-butoxycarbonyl-N-((2-(4-(3-(2-piperazin-1-ylethoxy)propanoyl)piperazin-1-yl)pyrimidin-5-yl)methyl)carbamate (1.0 equiv) in DCM is added succinic anhydride (1.2 equiv) and Et₃N (2.0 equiv). The reaction is stirred at room temperature until consumption of starting material, as determined by LCMS analysis. The reaction mixture is then concentrated under reduced pressure to give the crude product. The residue is purified by silica gel chromatography to afford the product.

### Building Block AM. 2-(4-(4-(4-(5-(((tert-butoxycarbonyl)amino)methyl)pyrimidin-2-yl)piperazin-1-yl)-4-oxobutyl)piperazin-1-yl)pyrimidine-5-carboxylic acid

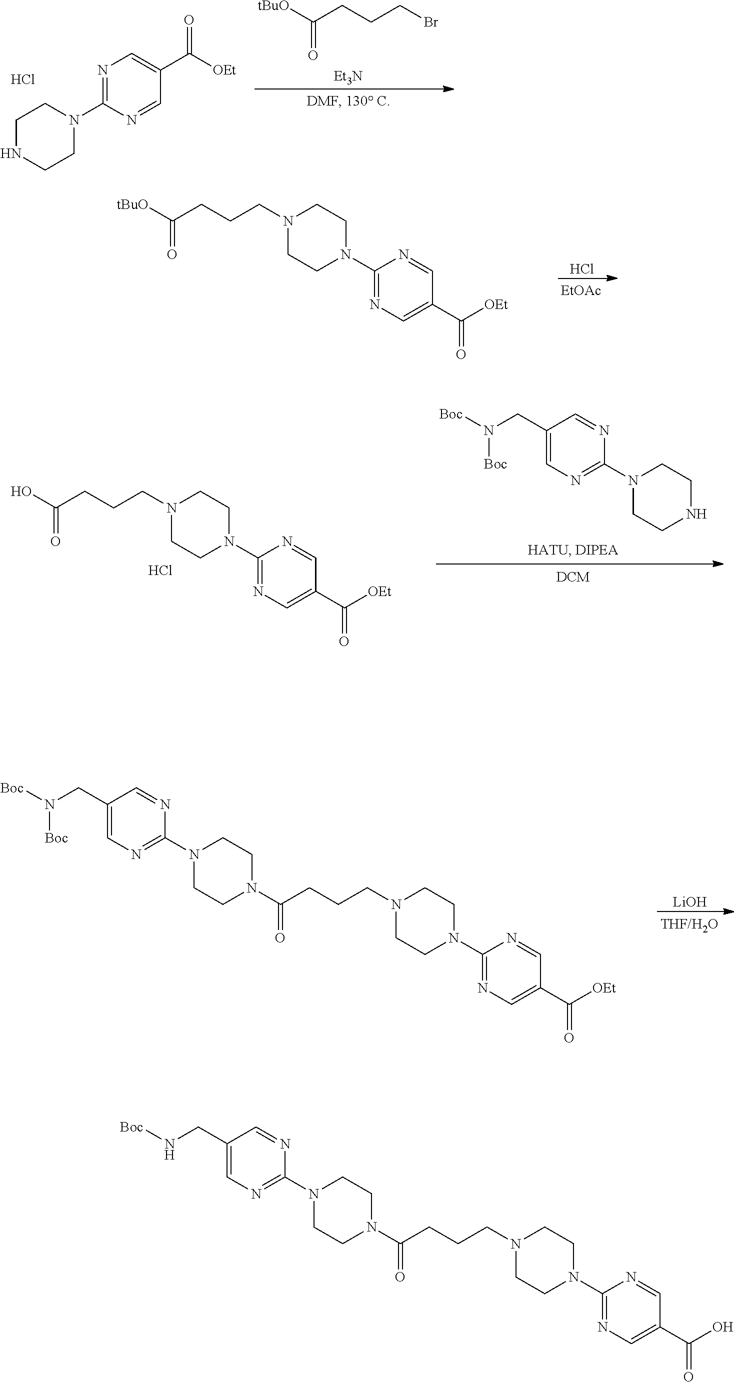

### Step 1: Synthesis of ethyl 2-(4-(4-(tert-butoxy)-4-oxobutyl)piperazin-1-yl)pyrimidine-5-carboxylate

To a solution of ethyl 2-(piperazin-1-yl)pyrimidine-5-carboxylate hydrochloride (10 g, 36.67 mmol, 1.0 equiv, HCl) and tert-butyl 4-bromobutanoate (8.18 g, 36.67 mmol, 1.0 equiv) in DMF (100 mL) was added Et₃N (15.31 mL, 110.00 mmol, 3.0 equiv). The mixture was stirred at 130° C. for 14 h. The mixture was then poured into H₂O (400 mL) and the solution was extracted with EtOAc (3×150 mL). The combined organic layer was washed with brine (200 mL), dried over Na₂SO₄ and concentrated under reduced pressure. The residue was purified by silica gel chromatography (5/1 to 1/1 petroleum ether/EtOAc) to give the product (9.5 g, 68.5% yield) as a yellow solid. LCMS (ESI) m/z: [M+H] calcd for C₁₉H₃₀N₄O₄: 379.24; found 379.2, 380.2.

### Step 2: Synthesis of 4-(4-(5-(ethoxycarbonyl)pyrimidin-2-yl)piperazin-1-yl)butanoic acid hydrochloride

To a solution of ethyl 2-(4-(4-(tert-butoxy)-4-oxobutyl)piperazin-1-yl)pyrimidine-5-carboxylate (9.5 g, 25.10 mmol, 1.0 equiv) in EtOAc (100 mL) was added HCV/EtOAc (500 mL). The mixture was stirred at room temperature for 10 h and then the solution was concentrated under reduced pressure to give the product (9.6 g, 96.8% yield) as a white solid. LCMS (ESI) m/z: [M+H] calcd for C₁₅H₂₂N₄O₄: 323.17; found 323.2.

### Step 3: Synthesis of ethyl 2-(4-(4-(4-(5-(((di-tert-butoxycarbonyl)amino)methyl)pyrimidin-2-yl)piperazin-1-yl)-4-oxobutyl)piperazin-1-yl)pyrimidine-5-carboxylic acid

To a solution of 4-(4-(5-(ethoxycarbonyl)pyrimidin-2-yl)piperazin-1-yl)butanoic acid hydrochloride (5 g, 15.51 mmol, 1.0 equiv) and tert-butyl N-tert-butoxycarbonyl-N-((2-piperazin-1-ylpyrimidin-5-yl)methyl)carbamate (6.10 g, 15.51 mmol, 1.0 equiv) in DMF (150 mL) was added DIPEA (8.11 mL, 46.53 mmol, 3.0 equiv) and HATU (7.08 g, 18.61 mmol, 1.2 equiv). The mixture was stirred at room temperature for 3 h and then the solution was poured into H₂O (600 mL). The aqueous layer was extracted with EtOAc (3×200 mL) and then the combined organic layer was washed with brine (100 mL), dried with Na₂SO₄ and concentrated under reduced pressure. The residue was purified by silica gel chromatography (50/1 to 15/1 DCM/MeOH) to give the product (6.3 g, 58.2% yield) as a yellow solid. LCMS (ESI) m/z: [M+H] calcd for C₄H₅₁N₉O₇: 698.40; found 698.6.

### Step 4: Synthesis of 2-(4-(4-(4-(5-(((tert-butoxycarbonyl)amino)methyl)pyrimidin-2-yl)piperazin-1-yl)-4-oxobutyl)piperazin-1-yl)pyrimidine-5-carboxylic acid

To a solution of ethyl 2-(4-(4-(4-(5-((bis(tert-butoxycarbonyl)amino)methyl)pyrimidin-2-yl)piperazin-1-yl)-4-oxo-butyl)piperazin-1-yl)pyrimidine-5-carboxylate (4.5 g, 6.45 mmol, 1.0 equiv) in EtOH (7 mL) and THF (28 mL) was added a solution of LiOH·H₂O (541.17 mg, 12.90 mmol, 2.0 equiv) in H₂O (7 mL). The mixture was stirred at 30° C. for 8 h, then additional LiOH·H₂O (541 mg, 12.90 mmol, 2.0 equiv) was added. After stirring for an additional 8 h at 30° C., the solution was concentrated under reduced pressure. H₂O (20 mL) was added and solution was adjusted to pH 3 with 1N HCl. The suspension was filtered and the solid dried under reduced pressure to give the product (3.2 g, 79.1% yield) as a white solid. LCMS (ESI) m/z: [M+H] calcd for C₂₇H₃₉N₉O₅: 570.32; found 570.3.

### Building Block AN. 2-(4-(2-(2-(4-(6-(tert-butoxycarbonyl)-5,6,7,8-tetrahydropyrido[4,3-d]pyrimidin-2-yl)piperazin-1-yl)ethoxy)ethyl)piperazin-1-yl)pyrimidine-5-carboxylic acid

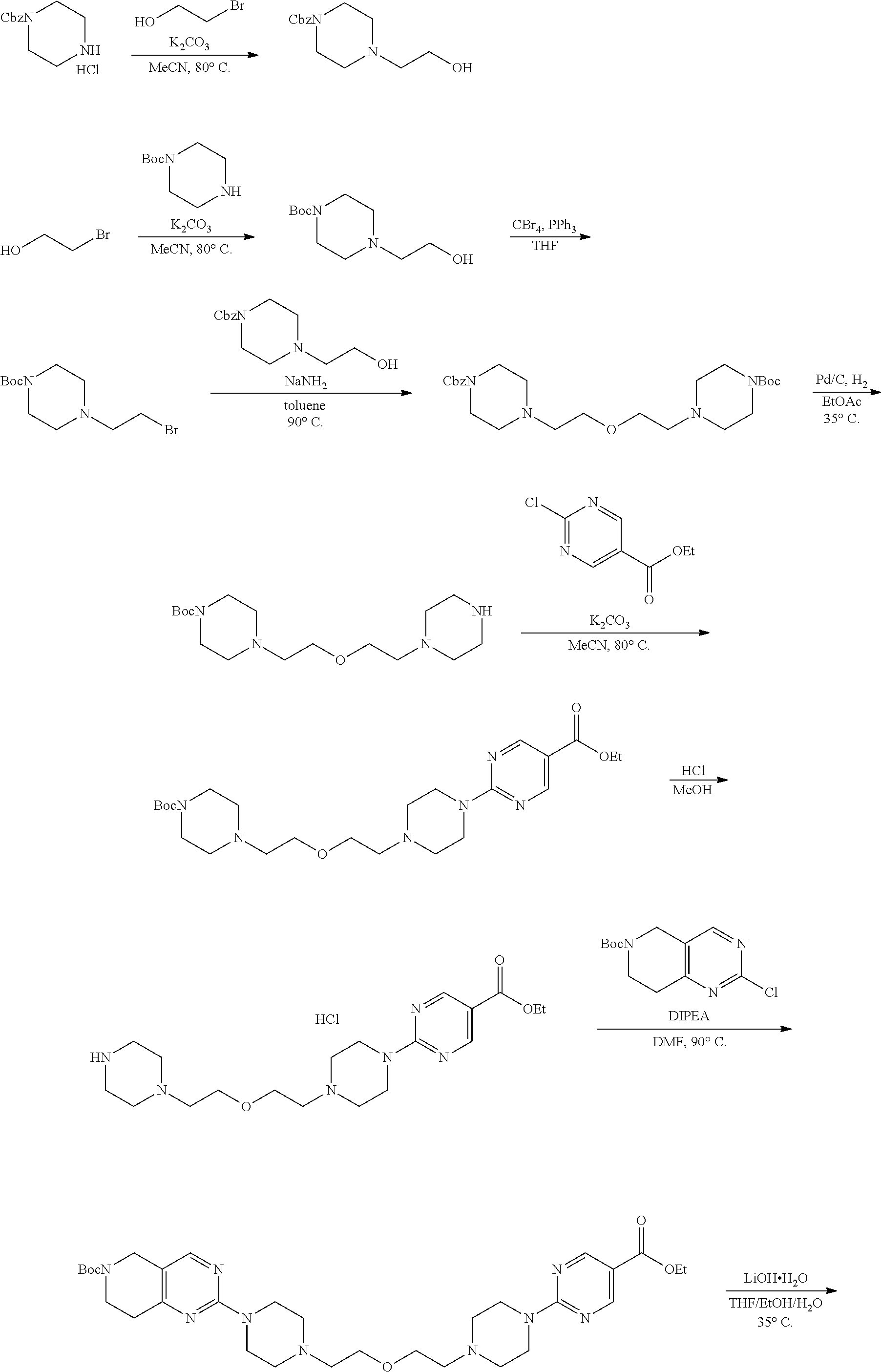

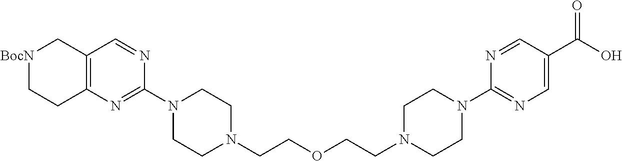 [SMILES: CC(C)(C)OC(=O)N1CCc2nc(N3CCN(CCOCCN4CCN(c5ncc(C(=O)O)cn5)CC4)CC3)ncc2C1]

### Step 1: Synthesis of benzyl 4-(2-hydroxyethyl)piperazine-1-carboxylate

To a solution of benzyl piperazine-1-carboxylate hydrochloride (41.09 g, 160.04 mmol, 1.0 equiv, HCl) in MeCN (200 mL) was added K₂CO₃ (66.36 g, 480.13 mmol, 3.0 equiv) and 2-bromoethanol (20 g, 160.04 mmol, 1.0 equiv). The reaction mixture was stirred at 80° C. for 16 h, at which point it was cooled to room temperature and filtered. The filter cake was washed with EtOAc (100 mL) and the filtrate then washed with H₂O (100 mL). The aqueous phase was extracted with EtOAc (3×50 mL) and the combined organic phases were washed with brine (50 mL), dried, and concentrated under reduced pressure. Purification by silica gel chromatography (5→25% MeOH/EtOAc) afforded the desired product as a yellow solid (20 g, 47% yield). LCMS (ESI) m/z: [M+H] calcd for C₁₄H₂₀N₂O₃: 265.16; found 264.9.

### Step 2: Synthesis of tert-butyl 4-(2-hydroxyethyl)piperazine-1-carboxylate

To a solution of tert-butyl piperazine-1-carboxylate (198.72 g, 1.07 mol, 1.0 equiv) in MeCN (1500 mL) was added 2-bromoethanol (240 g, 1.92 mol, 1.8 equiv) and K₂CO₃ (221.19 g, 1.60 mol, 1.5 equiv). The reaction mixture was stirred at 80° C. for 16 h, at which point the mixture was cooled to room temperature, filtered, and the filtrate was concentrated under reduced pressure. Purification by silica gel chromatography (0→14% MeOH/EtOAc) afforded the desired product as a white solid (146 g, 59% yield).

### Step 3: Synthesis of tert-butyl 4-(2-bromoethyl)piperazine-1-carboxylate

To a solution of tert-butyl 4-(2-hydroxyethyl)piperazine-1-carboxylate (45 g, 195.39 mmol, 1.0 equiv) in THF (600 mL) was added triphenylphosphine (97.38 g, 371.25 mmol, 1.9 equiv) and CBr₄ (116.64 g, 351.71 mmol, 1.8 equiv). The mixture was stirred at room temperature for 3 h. Two separate batches were combined, and the reaction mixture was filtered, and the filtrate concentrated under reduced pressure. Purification by silica gel chromatography (1→25% EtOAc/petroleum ether) afforded the desired product as a light-yellow solid (31 g, 27% yield).

### Step 4: Synthesis of benzyl 4-(2-(2-(4-(tert-butoxycarbonyl)piperazin-1-yl)ethoxy)ethyl)piperazine-1-carboxylate

To a solution of benzyl 4-(2-hydroxyethyl)piperazine-1-carboxylate (18 g, 68.10 mmol, 1.0 equiv) in toluene (200 mL) was added NaNH₂ (26.57 g, 680.99 mmol, 10.0 equiv), tert-Butyl 4-(2-bromoethyl)piperazine-1-carboxylate (25 g, 85.27 mmol, 1.25 equiv) was added and the mixture was heated to 90° C. for 18 h. The mixture was cooled to room temperature and poured into H₂O (700 mL) at 0° C. The aqueous phase was extracted with EtOAc (3×240 mL) and the combined organic phases were washed successively with H₂O (350 mL) and sat. brine (2×200 mL), dried, filtered, and concentrated under reduced pressure. Purification by silica gel chromatography (0→12% MeOH/EtOAc) afforded the desired product as a light-yellow oil (20 g, 62% yield).

### Step 5: Synthesis of tert-butyl 4-(2-(2-(piperazin-1-yl)ethoxy)ethyl)piperazine-1-carboxylate

To a solution of benzyl 4-(2-(2-(4-(tert-butoxycarbonyl)piperazin-1-yl)ethoxy)ethyl)piperazine-1-carboxylate (20 g, 41.96 mmol, 1.0 equiv) in EtOAc (180 mL) was added Pd/C (8 g, 10 wt. %). The suspension was degassed under reduced pressure and purged with H₂ three times. The mixture was stirred under H₂ (30 psi) at 35° C. for 12 h. The reaction mixture was then filtered, and the filtrate was concentrated under reduced pressure. Purification by silica gel chromatography (0→100% MeOH/EtOAc) afforded the desired product as a colorless oil (10.8 g, 75% yield).

### Step 6: Synthesis of ethyl 2-(4-(2-(2-(4-(tert-butoxycarbonyl)piperazin-1-yl)ethoxy)-ethyl)piperazin-1-yl)pyrimidine-5-carboxylate

To a solution of tert-butyl 4-(2-(2-(piperazin-1-yl)ethoxy)ethyl)piperazine-1-carboxylate (10.8 g, 31.54 mmol, 1.0 equiv) in MeCN (100 mL) was added K₂CO₃ (13.08 g, 94.61 mmol, 3.0 equiv) and ethyl 2-chloropyrimidine-5-carboxylate (5.88 g, 31.54 mmol, 1.0 equiv). The mixture was stirred at 80° C. for 12 h, at which point the reaction was cooled to room temperature, filtered, and the filtrate concentrated under reduced pressure. Purification by silica gel chromatography (0→9% MeOH/DCM) afforded the desired product as a white solid (13.6 g, 85% yield).

### Step 7: Synthesis of 2-(4-(2-(2-(4-(6-(tert-butoxycarbonyl)-5,6,7,8-tetrahydropyrido-[4,3-d]pyrimidin-2-yl)piperazin-1-yl)ethoxy)ethyl)piperazin-1-yl)pyrimidine-5-carboxylic acid

To a solution of ethyl 2-(4-(2-(2-(4-(tert-butoxycarbonyl)piperazin-1-yl)ethoxy)ethyl)piperazin-1-yl)pyrimidine-5-carboxylate (13.6 g, 27.61 mmol, 1.0 equiv) in MeOH (50 mL) was added a solution of HCl in MeOH (4 M. 150 mL, 21.7 equiv). The reaction was stirred at room temperature for 4 h, at which point the mixture was concentrated under reduced pressure to afford the crude desired product as a white solid (13.8 g, 4HCl) that was taken directly onto the next step. LCMS (ESI) m/z: [M+H] calcd for C₉H₃₂N₆O₃: 393.26; found 393.3.

### Step 8: Synthesis of tert-butyl 2-(4-(2-(2-(4-(5-(ethoxycarbonyl)pyrimidin-2-yl)-piperazin-1-yl)ethoxy)ethyl)piperazin-1-yl)-7,8-dihydropyrido[4,3-d]pyrimidine-6(5H)-carboxylate

To a stirred solution of 2-(4-(2-(2-(4-(6-(tert-butoxycarbonyl)-5,6,7,8-tetrahydropyrido[4,3-d]pyrimidin-2-yl)piperazin-1-yl)ethoxy)ethyl)piperazin-1-yl)pyrimidine-5-carboxylic acid (10.2 g, 18.95 mmol, 1.0 equiv, 4HCl) and DIPEA (16.50 mL, 94.74 mmol, 5.0 equiv) in DMF (100 mL) was added tert-butyl 2-chloro-7,8-dihydropyrido[4,3-d]pyrimidine-6(5H)-carboxylate (5.11 g, 18.95 mmol, 1.0 equiv). The reaction mixture was stirred at 90° C. for 12 h. The reaction mixture was then cooled to room temperature and added to EtOAc (200 mL) and H₂O (400 mL). The aqueous phase was extracted with EtOAc (2×100 mL) and the combined organic phases were washed with aqueous NH₄Cl (4×100 mL), brine (2×100 mL), dried, filtered and concentrated under reduced pressure. Purification by silica gel chromatography (0→9% MeOH/DCM) afforded the desired product as a white solid (5.4 g, 45% yield). LCMS (ESI) m/z: [M+H] calcd for C₃₁H₄₇N₉O₅: 626.38; found 626.3.

### Step 9: Synthesis of 2-(4-(2-(2-(4-(6-(tert-butoxycarbonyl)-5,6,7,8-tetrahydropyrido[4,3-d]pyrimidin-2-yl)piperazin-1-yl)ethoxy)ethyl)piperazin-1-yl)pyrimidine-5-carboxylic acid

To a solution of tert-butyl 2-(4-(2-(2-(4-(5-(ethoxycarbonyl)pyrimidin-2-yl)piperazin-1-yl)ethoxy)ethyl)piperazin-1-yl)-7,8-dihydropyrido[4,3-d]pyrimidine-6(5H)-carboxylate (5.4 g, 8.63 mmol, 1.0 equiv) in THF (50 mL), EtOH (20 mL), and H₂O (20 mL) was added LiOH·H₂O (1.09 g, 25.89 mmol, 3.0 equiv). The reaction mixture was stirred at 35° C. for 12 h, at which point the mixture was concentrated under reduced pressure to remove THF and EtOH. The aqueous phase was neutralized to pH=7 with 0.5N HCl and concentrated under reduced pressure. Purification by reverse phase chromatography afforded the desired product as a white solid (4.72 g, 92% yield). LCMS (ESI) m/z: [M+H] calcd for C₂₉H₄₃N₉O₅: 598.35; found 598.3.

### Building Block AO. 1′-[(tert-butoxy)carbonyl]-[1,4′-bipiperidine]-4-carboxylic acid

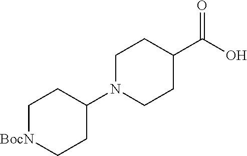 [SMILES: CC(C)(C)OC(=O)N1CCC(N2CCC(C(=O)O)CC2)CC1]

At the time of this application this building block was commercially available (CAS #201810-59-5).

### Building Block AP. 2-((2-(piperazin-1-yl)pyrimidin-5-yl)methyl)isoindoline-1,3-dione hydrochloride salt

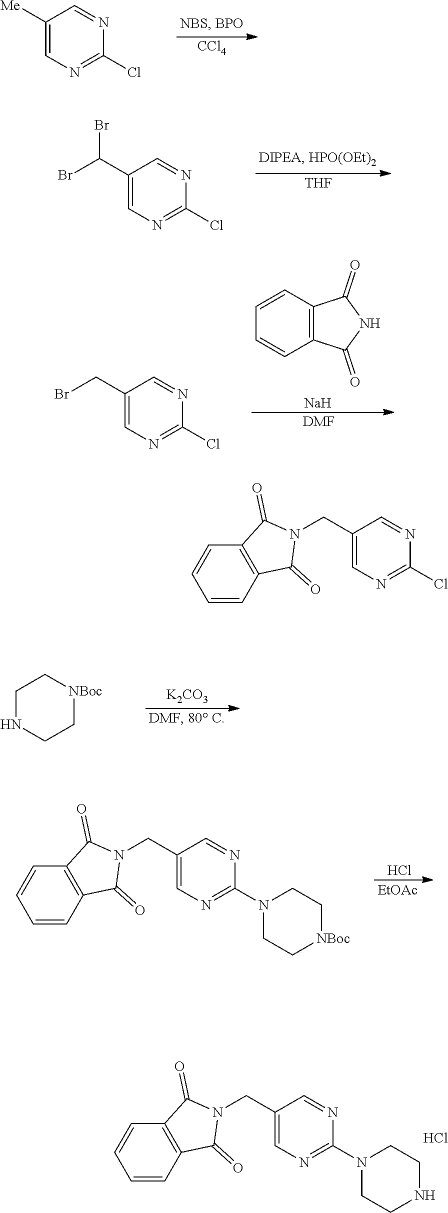

### Step 1: Synthesis of 2-chloro-5-(dibromomethyl)pyrimidine

To a solution of 2-chloro-5-methylpyrimidine (100 g, 777.85 mmol, 1.0 equiv) in CCl₄ (1200 mL) was added NBS (304.58 g, 1.71 mol, 2.2 equiv) and AIBN (51.09 g, 311.14 mmol, 0.4 equiv). The mixture was stirred at 80° C. for 16 h. The reaction solution was then cooled to room temperature, filtered, and the filtrate was poured into H₂O (1500 mL). The solution was diluted with DCM (3×250 mL) and the organic layer washed with brine (300 mL), dried with anhydrous Na₂SO₄, filtered and concentrated under reduced pressure to give the crude product as a brown oil, which was used directly in the next step.

### Step 2: Synthesis of 5-(bromomethyl)-2-chloropyrimidine

To a solution of 2-chloro-5-(dibromomethyl)pyrimidine (229 g, 799.72 mmol, 1.0 equiv) in THF (600 mL) was added DIPEA (111.44 mL, 639.77 mmol, 0.8 equiv) and 1-ethoxyphosphonoyloxyethane (82.57 mL, 639.77 mmol, 0.8 equiv). The mixture was stirred at room temperature for 19 h. The mixture was then poured into H₂O (1200 mL) and the aqueous phase was extracted with EtOAc (3×300 mL). The combined organic phase was washed with brine (300 mL), dried with anhydrous Na₂SO₄, filtered and concentrated under reduced pressure. The residue was purified by silica gel chromatography (1/0 to 0/1 petroleum ether/EtOAc) to give the product as a brown oil, which was used directly for the next step.

### Step 3: Synthesis of 2-((2-chloropyrimidin-5-yl)methyl)isoindoline-1,3-dione

To a mixture of isoindoline-1,3-dione (15 g, 101.95 mmol, 1.0 equiv) in DMF (126 mL) was added NaH (4.89 g, 122.34 mmol, 60 wt. %, 1.2 equiv) at 0° C. The mixture was stirred at 0° C. for 30 min, then a solution of 5-(bromomethyl)-2-chloro-pyrimidine (30.21 g, 101.95 mmol, 1.0 equiv) in DMF (24 mL) was added dropwise to the above mixture at room temperature. The mixture was stirred at room temperature for 2 h and was then cooled to 0° C. and quenched with sat. NH₄Cl (600 mL). The suspension was filtered and the solid dried under reduced pressure to give the crude product (27.4 g, 98.2% yield) as a grey solid, which was used directly in the next step. LCMS (ESI) m/z: [M+H] calcd for C₁₃H₈ClN₃O₂: 274.04; found 274.0.

### Step 4: Synthesis of tert-butyl 4-(5-((1,3-dioxoisoindolin-2-yl)methyl)pyrimidin-2-yl)piperazine-1-carboxylate

To a solution of 2-((2-chloropyrimidin-5-yl)methyl)isoindoline-1,3-dione (27 g, 98.66 mmol, 1.0 equiv) and tert-butyl piperazine-1-carboxylate (20.21 g, 108.52 mmol, 1.1 equiv) in DMF (270 mL) was added K₂CO₃ (34.09 g, 246.64 mmol, 2.5 equiv). The mixture was stirred at 80° C. for 3 h and then the reaction was cooled to room temperature and poured into H₂O (1200 mL). The suspension was filtered and the solid was dried under reduced pressure to give the crude product (35.58 g, 85.2% yield) as a white solid, which was used directly in the next step.

### Step 5: Synthesis of 2-((2-(piperazin-1-yl)pyrimidin-5-yl)methyl)isoindoline-1,3-dione

A solution of tert-butyl 4-(5-((1,3-dioxoisoindolin-2-yl)methyl)pyrimidin-2-yl)piperazine-1-carboxylate (15 g, 35.42 mmol, 1 equiv) in HCl/EtOAc (150 mL) was stirred at room temperature for 2 h. The mixture was filtered and then the filter cake was washed with EtOAc (20 mL) and dried under reduced pressure to give the product (42.53 g, 92.5% yield) as a white solid.

### Building Block AQ. 2-[(2-{4-[2-(3-{4-[5-({bis[(tert-butoxy)carbonyl]amino}methyl)pyrimidin-2-yl]piperazin-1-yl}-3-oxopropoxy)ethyl]piperazin-1-yl}-2-oxoethyl)(methyl)amino]acetic acid

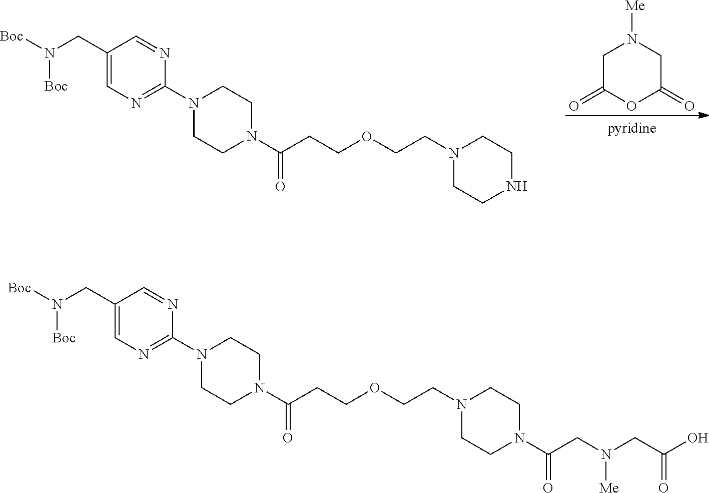 [SMILES: CC(C)(C)OC(=O)N(Cc1cnc(N2CCN(C(=O)CCOCCN3CCNCC3)CC2)nc1)C(=O)OC(C)(C)C ; CN1CC(=O)OC(=O)C1 ; CN(CC(=O)O)CC(=O)N1CCN(CCOCCC(=O)N2CCN(c3ncc(CN(C(=O)OC(C)(C)C)C(=O)OC(C)(C)C)cn3)CC2)CC1]

To a solution of tert-butyl N-[(tert-butoxy)carbonyl]-N-{[2-(4-(3-[2-(piperazin-1-yl)ethoxy]propanoyl)piperazin-1-yl)pyrimidin-5-yl]methyl}carbamate (30) mg, 519 μmol, 1.0 equiv) in pyridine (8 mL) at 0° C. was added 4-methylmorpholine-2,6-dione (80.3 mg, 622 μmol, 1.2 equiv). The reaction mixture was stirred at 0° C. for 1 h and then warmed to room temperature and stirred for an additional 12 h. The solvent was concentrated under reduced pressure and the solid was partitioned between DCM and H₂O. The organic layer was separated, dried over MgSO₄ and the solvent was concentrated under reduced pressure to give the product (23.0 mg, 6.28% yield). LCMS (ESI) m/z: [M+H] calcd for C₃₃H₅₄N₈O₉: 707.41; found 707.4.

### Building Block AR. 2-(4-(2-(3-(4-(6-(tert-butoxycarbonyl)-5,6,7,8-tetrahydropyrido[4,3-d]pyrimidin-2-yl)piperazin-1-yl)-3-oxopropoxy)ethyl)piperazin-1-yl)pyrimidine-5-carboxylic acid

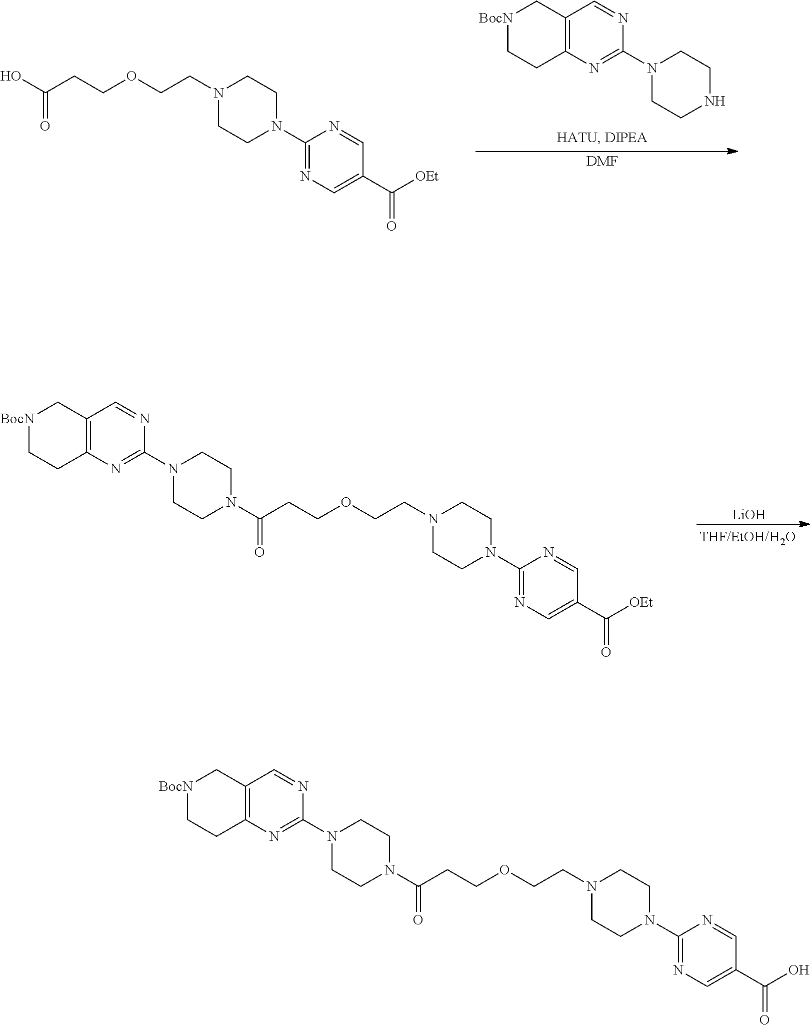

### Step 1: Synthesis of tert-butyl 2-(4-(3-(2-(4-(5-(ethoxycarbonyl)pyrimidin-2-yl)piperazin-1-yl)ethoxy)propanoyl)piperazin-1-yl)-7,8-dihydropyrido[4,3-d]pyrimidine-6(5H)-carboxylate

To a solution of 3-(2-(4-(5-(ethoxycarbonyl)pyrimidin-2-yl)piperazin-1-yl)ethoxy) propanoic acid (6 g, 12.86 mmol, 1.0 equiv, TFA) in DMF (55 mL) was added HATU (6.36 g, 16.72 mmol, 1.3 equiv) and DIPEA (11.20 mL, 64.32 mmol, 5.0 equiv). After 0.5 h, tert-butyl 2-(piperazin-1-yl)-7,8-dihydropyrido[4,3-d]pyrimidine-6(5H)-carboxylate (4.11 g, 12.86 mmol, 1.0 equiv) was added. The mixture was stirred for 3 h, at which point it was filtered and the solid cake was dried under reduced pressure to afford the desired product as a white solid (7.5 g, 89% yield). LCMS (ESI) m/z: [M+H] calcd for C₃₂H₄₇N₉O₆: 654.37; found 654.4.

### Step 2: Synthesis of 2-(4-(2-(3-(4-(6-(tert-butoxycarbonyl)-5,6,7,8-tetrahydropyrido[4,3-d]pyrimidin-2-yl)piperazin-1-yl)-3-oxopropoxy)ethyl)piperazin-1-yl)pyrimidine-5-carboxylic acid

To a solution of tert-butyl 2-(4-(3-(2-(4-(5-(ethoxycarbonyl)pyrimidin-2-yl)piperazin-1-yl)ethoxy)propanoyl)piperazin-1-yl)-7,8-dihydropyrido[4,3-d]pyrimidine-6(5H)-carboxylate (7.2 g, 11.01 mmol, 1.0 equiv) in THF (72 mL), EtOH (36 mL) and H₂O (36 mL) was added LiOH·H₂O (1.85 g, 44.05 mmol, 4.0 equiv). The reaction mixture was stirred at room temperature for 2.5 h, at which point the mixture was filtered and the filtrate was concentrated under reduced pressure to remove THF and EtOH. The aqueous phase was neutralized to pH=7 with 1N HCl, and then concentrated under reduced pressure. Purification by reverse phase chromatography (30% MeCN/H₂O) afforded the desired product as a white solid (3.85 g, 54% yield). LCMS (ESI) m/z: [M+H] calcd for C₃₀H₄₃N₉O₆: 626.34; found 626.3.

### Building Block AS. 2-(4-(2-(2-(3-(4-(5-((di-tert-butoxycarbonylamino)methyl)pyrimidin-2-yl)piperazin-1-yl)-3-oxo-propoxy)ethoxy)ethyl)piperazin-1-yl)pyrimidine-5-carboxylic acid

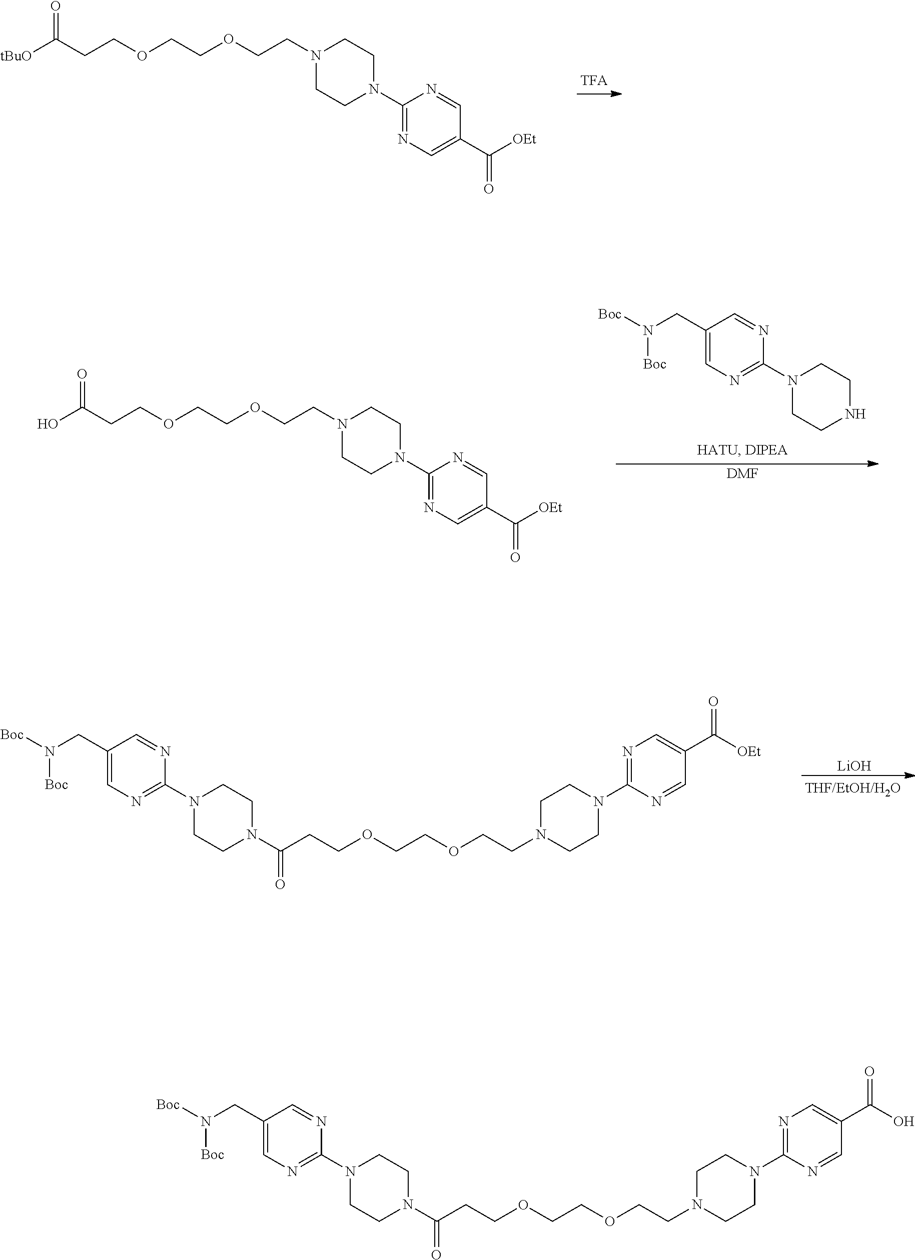

### Step 1: Synthesis of 3-(2-(2-(4-(5-(ethoxycarbonyl)pyrimidin-2-yl)piperazin-1yl)ethoxy)ethoxy)propanoic acid

A solution of ethyl 2-(4-(2-(2-(3-(tert-butoxy)-3-oxopropoxy)ethoxy)ethyl)piperazin-1-yl)pyrimidine-5-carboxylate (4 g, 8.84 mmol, 1.0 equiv) in TFA (12.29 mL, 166.00 mmol, 18.8 equiv) was stirred at room temperature for 3 h. The reaction mixture was concentrated under reduced pressure. Purification by silica gel chromatography (0→20% MeOH/EtOAc) afforded the desired product as a brown oil (4.35 g, 95% yield, TFA salt).

### Step 2: Synthesis of ethyl 2-(4-(2-(2-(3-(4-(5-((bis(tert-butoxycarbonyl)amino)methyl)pyrimidin-2-yl)piperazin-1-yl)-3-oxopropoxy)ethoxy)ethyl)piperazin-1-yl)pyrimidine-5-carboxylate

To a solution of 3-(2-(2-(4-(5-ethoxycarbonylpyrimidin-2-yl)piperazin-1-yl)ethoxy)ethoxy)propanoic acid (3.8 g, 7.44 mmol, 1.0 equiv, TFA) in DCM (30 mL) was added HATU (4.25 g, 11.17 mmol, 1.5 equiv) and DIPEA (6.48 mL, 37.22 mmol, 5.0 equiv). The reaction was stirred at room temperature for 30 min, and then tert-butyl N-tert-butoxycarbonyl-N-((2-piperazin-1ylpyrimidin-5-yl)methyl)carbamate (2.93 g, 7.44 mmol, 1.0 equiv) was added. The mixture was stirred at room temperature for 3.5 h, at which point the reaction mixture was concentrated under reduced pressure. Purification by silica gel chromatography (0→20% MeOH/EtOAc) afforded the desired product as a brown oil (4.14 g, 70% yield).

### Step 3: Synthesis of 2-(4-(2-(2-(3-(4-(5-((di-tert-butoxycarbonylamino)methyl)pyrimidin-2-yl)piperazin-1-yl)-3-oxo-propoxy)ethoxy)ethyl)piperazin-1-yl)pyrimidine-5-carboxylic acid

To a solution of ethyl 2-(4-(2-(2-(3-(4-(5-((bis(tert-butoxycarbonyl)amino)methyl)pyrimidin-2-yl)piperazin-1-yl)-3-oxo-propoxy)ethoxy)ethyl)piperazin-1-yl)pyrimidine-5-carboxylate (1.4 g, 1.81 mmol, 1.0 equiv) in THF (28 mL), EtOH (14 mL) and H₂O (14 mL) was added LiOH·H₂O (304.44 mg, 7.25 mmol, 4.0 equiv). The mixture was stirred at 40° C. for 30 min, at which point the reaction mixture was concentrated under reduced pressure. Purification by reverse phase chromatography (10→40% MeCN/H₂O) afforded the desired product as a yellow solid (500 mg, 43% yield).

### Building Block AT. 2-{4-[2-(2-{4-[5-({[(tert-butoxy)carbonyl]amino}methyl)pyrimidin-2-yl]piperazin-1-yl}ethoxy)ethyl]piperazin-1-yl}pyrimidine-5-carboxylic acid

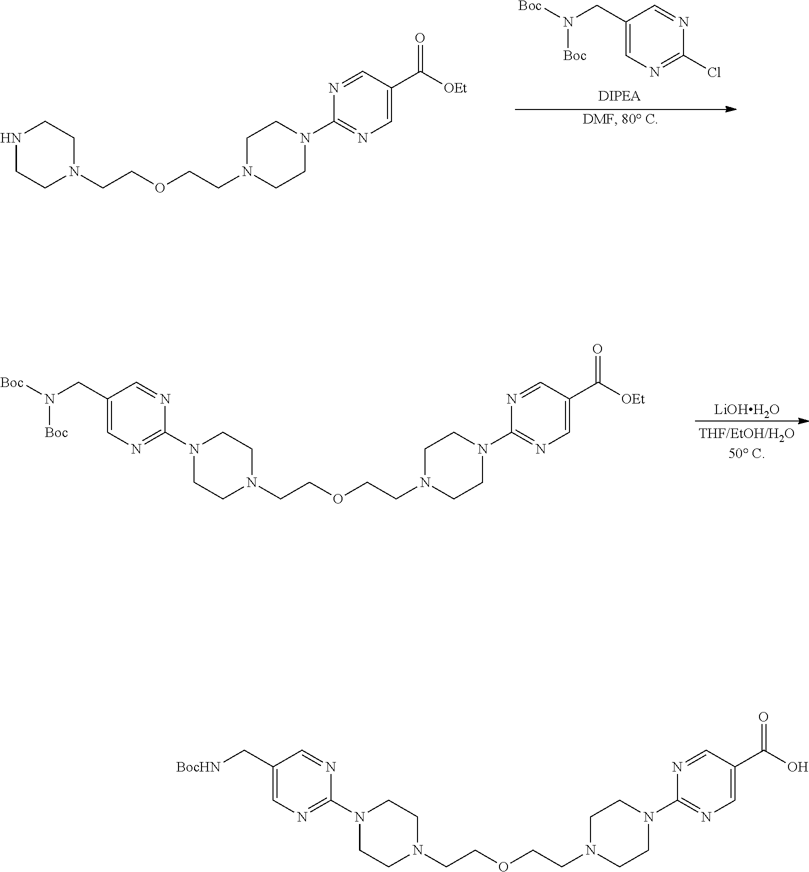 [SMILES: CCOC(=O)c1cnc(N2CCN(CCOCCN3CCNCC3)CC2)nc1 ; CC(C)(C)OC(=O)N(Cc1cnc(Cl)nc1)C(=O)OC(C)(C)C ; "DMF, 80° C." ; CCOC(=O)c1cnc(N2CCN(CCOCCN3CCN(c4ncc(CN(C(=O)OC(C)(C)C)C(=O)OC(C)(C)C)cn4)CC3)CC2)nc1 ; "LiOH·H2O" ; CC(C)(C)OC(=O)NCc1cnc(N2CCN(CCOCCN3CCN(c4ncc(C(=O)O)cn4)CC3)CC2)nc1]

### Step 1: Synthesis of ethyl 2-(4-(2-(2-(4-(5-(((di-tert-butoxycarbonyl)amino)methyl)pyrimidin-2-yl)piperazin-1-yl)ethoxy)ethyl)piperazin-1-yl)pyrimidine-5-carboxylate

To a solution of ethyl 2-(4-(2-(2-(piperazin-1-yl)ethoxy)ethyl)piperazin-1-yl)pyrimidine-5-carboxylate hydrochloride (7.3 g, 13.56 mmol, 1.0 equiv, 4HCl) in DMF (75 mL) was added DIPEA (14.17 mL, 81.36 mmol, 6.0 equiv) and tert-butyl-N-tert-butoxycarbonyl-N-[(2-chloropyrimidin-5-yl)methyl]carbamate (5.59 g, 16.27 mmol, 1.2 equiv). The mixture was stirred at 80° C. for 12 h. The mixture was then cooled to room temperature and poured into H₂O (300 mL). The aqueous phase was extracted with EtOAc (3×80 mL). The combined organic phases were washed with sat. NH₄Cl (4×80 mL) and brine (150 mL), dried, filtered and the filtrate was concentrated under reduced pressure. Purification by silica gel chromatography (0%→17% MeOH/EtOAc) afforded the desired product (7.7 g, 81.1% yield) as a light yellow oil. LCMS (ESI) m/z: [M+Na] calcd for C₃₄H₅₃N₉O₇: 722.40; found 722.4.

### Step 2: Synthesis of 2-(4-(2-(2-(4-(5-(((tert-butoxycarbonyl)amino)methyl)pyrimidin-2-yl)piperazin-1-yl)ethoxy)ethyl)piperazin-1-yl)pyrimidine-5-carboxylic acid

To a solution of ethyl 2-(4-(2-(2-(4-(5-(((di-tert-butoxycarbonyl)amino)methyl)pyrimidin-2-yl)piperazin-1-yl)ethoxy)ethyl)piperazin-1-yl)pyrimidine-5-carboxylate (7.7 g, 11.00 mmol, 1.0 equiv) in THF (80 mL), EtOH (20 mL), and H₂O (40 mL) was added LiOH·H₂O (2.31 g, 55.01 mmol, 5.0 equiv). The mixture was stirred at 50° C. for 26 h. The mixture was then concentrated under reduced pressure to remove THF and EtOH. The aqueous phase was neutralized with 0.5 N HCl, and concentrated under reduced pressure. Purification by reverse phase chromatography afforded the desired product (4.67 g, 74.3% yield) as a white solid. LCMS (ESI) m/z: [M−H] calcd for C₂₇H₄₁N₉O₅: 570.31; found 570.3.

### Building Block AU. (R)-tert-butyl 4-(5-(((tert-butoxycarbonyl-N-tert-butoxycarbonyl)amino)methyl)pyrimidin-2-yl)piperazine-2-carboxylate

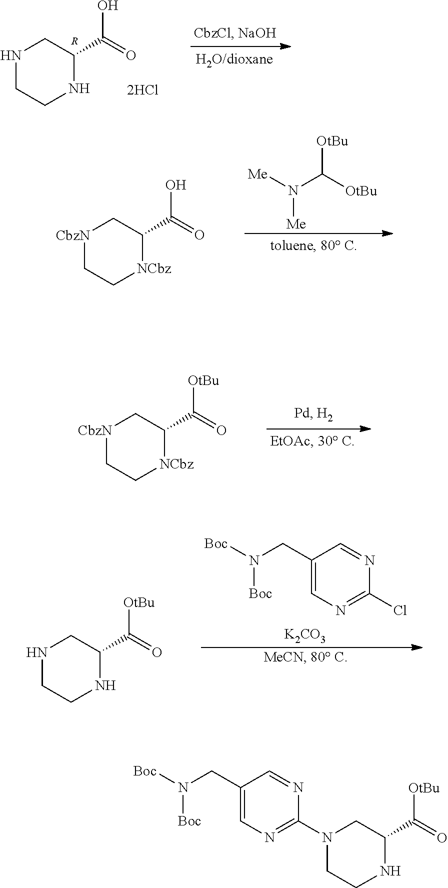

Step 1: Synthesis of (R)-1,4-bis((benzyloxy)carbonyl)piperazine-2-carboxylic acid

To two separate batches containing a solution (2R)-piperazine-2-carboxylic acid (70 g, 344.71 mmol, 1 equiv, 2HCl) in H₂O (700 mL) and dioxane (1120 mL) was added 50% aq. NaOH until pH=11. Benzyl chloroformate (156.82 mL, 1.10 mol, 3.2 equiv) was added and the reaction was stirred at room temperature for 12 h. The two reaction mixtures were combined and H₂O (1200 mL) was added. The aqueous layer was extracted with MTBE (3×1000 mL), adjusted to pH=2 with con. HCl, and then extracted with EtOAc (2×1000 mL). The combined organic phases were dried, filtered, and concentrated under reduced pressure to afford the desired product (280 g, 86% yield). LCMS (ESI) m/z: [M+H] calcd for C₂₁H₂₂N₂O₆: 399.16; found 399.0.

### Step 2: Synthesis of (R)-1,4-dibenzyl 2-tert-butyl piperazine-1,2,4-tricarboxylate

To a solution of (R)-1,4-bis((benzyloxy)carbonyl)piperazine-2-carboxylic acid (70 g, 175.70 mmol, 1.0 equiv) in toluene (700 mL) at 80° C. was added 1,1-di-tert-butoxy-N,N-dimethyl-methanamine (80.04 mL, 333.83 mmol, 1.9 equiv). The reaction was stirred at 80° C. for 2 h, at which point it was cooled to room temperature and partitioned between EtOAc (300 mL) and H₂O (500 mL). The aqueous layer was extracted with EtOAc (2×500 mL) and the combined organic layers were dried, filtered, and concentrated under reduced pressure. Purification by silica gel chromatography (0→25 EtOAc/petroleum ether) afforded the desired product as a white solid (50 g, 57% yield). LCMS (ESI) m/z: [M+Na] calcd for C₂₅H₃₀N₂O₆: 477.20; found 476.9.

### Step 3: Synthesis of (R)-tert-butyl piperazine-2-carboxylate

To a solution of (R)-1,4-dibenzyl 2-tert-butyl piperazine-1,2,4-tricarboxylate (50 g, 110.01 mmol, 1 equiv) in EtOAc (20 mL) was added Pd/C (15 g, 10 wt. %). The suspension was degassed under reduced pressure and purged with H₂ three times. The suspension was stirred under H₂ (30 psi) at 30° C. for 4 h. The reaction mixture was then filtered, the residue was washed with MeOH (5×200 mL), and the filtrate concentrated under reduced pressure to afford the desired product as a yellow oil (17 g, 81% yield). LCMS (ESI) m/z: [M+H] calcd for C₉H₁₈N₂O₂: 187.15; found 187.1.

### Step 4: Synthesis of (R)-tert-butyl 4-(5-(((tert-butoxycarbonyl-N-tert-butoxycarbonyl)amino)methyl)pyrimidin-2-yl)piperazine-2-carboxylate

To a suspension of (R)-tert-butyl piperazine-2-carboxylate (8 g, 23.27 mmol, 1.0 equiv) and tert-butyl-N-tert-butoxycarbonyl ((2-chloropyrimidin-5-yl)methyl)carbamate (5.20 g, 27.92 mmol, 1.2 equiv) in MeCN (100 mL) was added K₂CO₃ (6.43 g, 46.54 mmol, 2.0 equiv). The reaction mixture was heated to 80° C. for 12 h, at which point it was cooled to room temperature, filtered, and the filtrate was concentrated under reduced pressure. Purification by silica gel chromatography (0→100% EtOAc/petroleum ether) afforded the desired product as a yellow solid (9.2 g, 73% yield). LCMS (ESI) m/z: [M+H] calcd for C₂₄H₃₉N₅O₆:494.30; found 494.1.

### Building Block AV. (S)-tert-butyl 4-(5-(((tert-butoxycarbonyl-N-tert-butoxycarbonyl)amino)methyl)pyrimidin-2-yl)piperazine-2-carboxylate

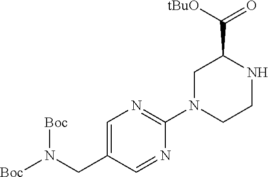 [SMILES: CC(C)(C)OC(=O)[C@@H]1CN(c2ncc(CN(C(=O)OC(C)(C)C)C(=O)OC(C)(C)C)cn2)CCN1]

This building block is prepared by a process similar to that for Building block AU by utilizing (2S)-piperazine-2-carboxylic acid.

### Building Block AW. (R)-2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylic acid

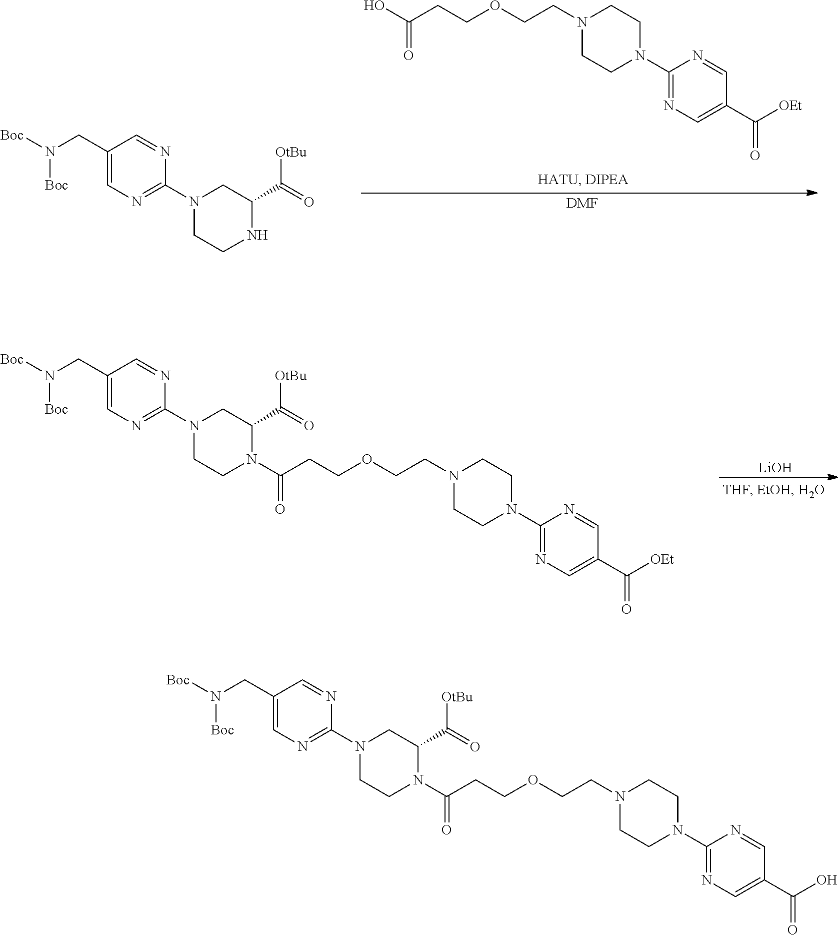

### Step 1: Synthesis of (R)-ethyl 2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylate

To a solution of (R)-tert-butyl 4-(5-(((tert-butoxycarbonyl-N-tert-butoxycarbonyl)amino)methyl)pyrimidin-2-yl)piperazine-2-carboxylate (5.3 g, 11.36 mmol, 1.0 equiv. TFA) in DCM (80 mL) was added HATU (6.48 g, 17.05 mmol, 1.5 equiv) and DIPEA (7.92 mL, 45.45 mmol, 4.0 equiv). The reaction was stirred at room temperature for 30 min and then tert-butyl (2R)-4-(5-((bis(tert-butoxycarbonyl)amino)methyl)pyrimidin-2-yl)piperazine-2-carboxylate (5.61 g, 11.36 mmol, 1.0 equiv) was added. The mixture was stirred for 1 h, at which point sat. NH₄Cl (80 mL) was added. The organic phase was washed with sat. NH₄Cl (5×80 mL), dried, filtered, and concentrated under reduced pressure. Purification by silica gel chromatography (0→9% MeOH/EtOAc) afforded the desired product as a yellow solid (8.4 g, 85% yield).

### Step 2: Synthesis of (R)-2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylic acid

To two separate batches containing a solution a solution of (R)-ethyl 2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylate (3.4 g, 4.11 mmol, 1.0 equiv) in THF (16 mL), EtOH (8 mL) and H₂O (8 mL) was added LiOH·H₂O (344.61 mg, 8.21 mmol, 2.0 equiv). The mixture was stirred at room temperature for 2 h. The two reaction mixtures were then combined and were adjusted to pH=7 with 1N HCl. The solution was concentrated under reduced pressure to remove THF and EtOH. The solution was then filtered, and the resulting solid was purified by reverse phase chromatography to afford the desired product as a white solid (4 g, 59% yield). LCMS (ESI) m/z: [M+H] calcd for C₃₈H₅₇N₉O₁₀: 800.43; found 800.3.

### Building Block AX. (S)-2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylic acid

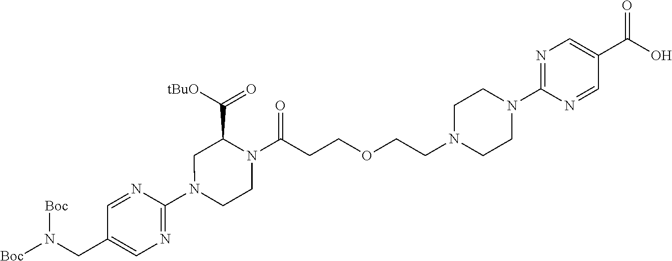 [SMILES: CC(C)(C)OC(=O)[C@@H]1CN(c2ncc(CN(C(=O)OC(C)(C)C)C(=O)OC(C)(C)C)cn2)CCN1C(=O)CCOCCN1CCN(c2ncc(C(=O)O)cn2)CC1]

This building block is prepared from Building block AV by a process similar to that for Building block AW.

### Building Block AY. 1′-(2-(3-(4-(5-(((tert-butoxycarbonyl)amino)methyl)pyrimidin-2-yl) piperazin-1-yl)-3-oxopropoxy)ethyl)-[1,4′-bipiperidine]-4-carboxylic acid

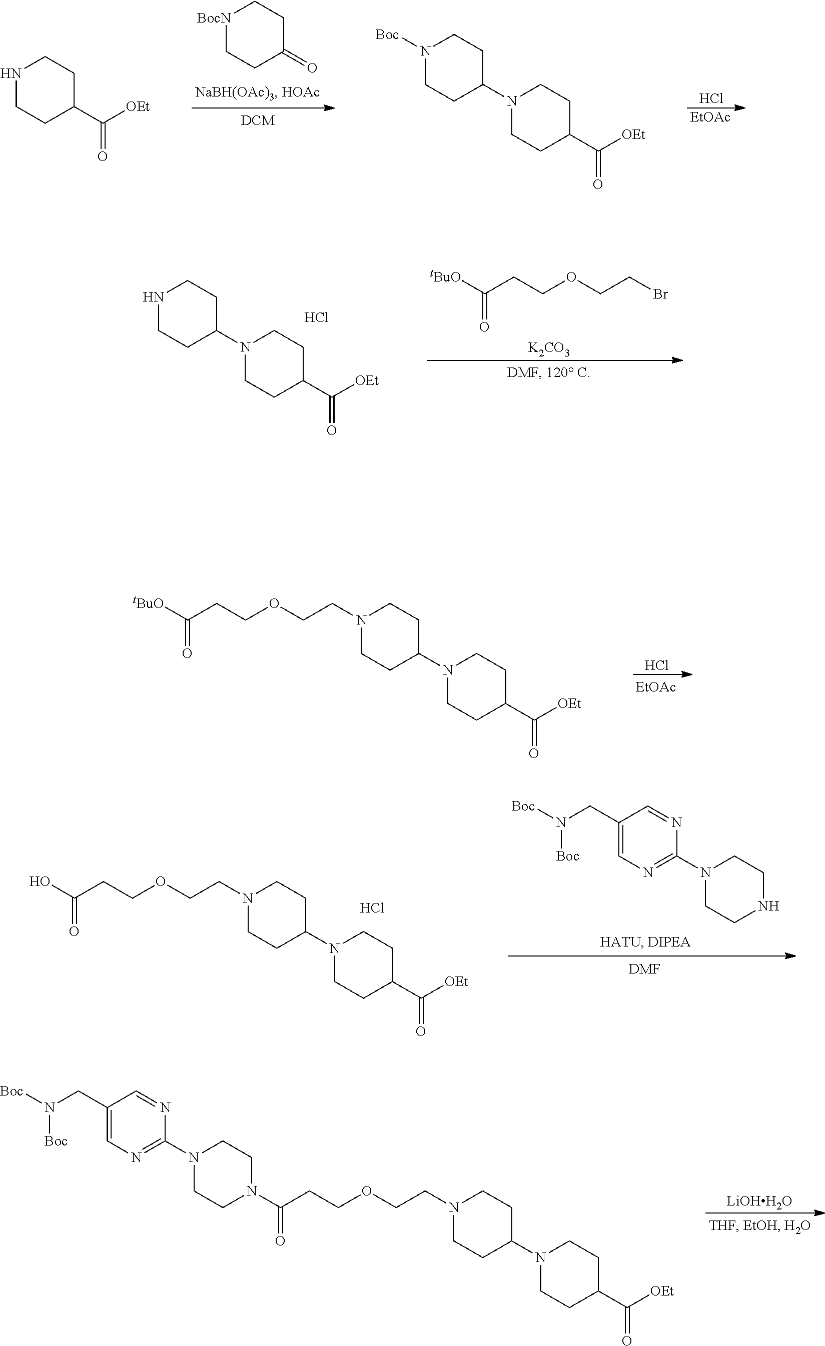

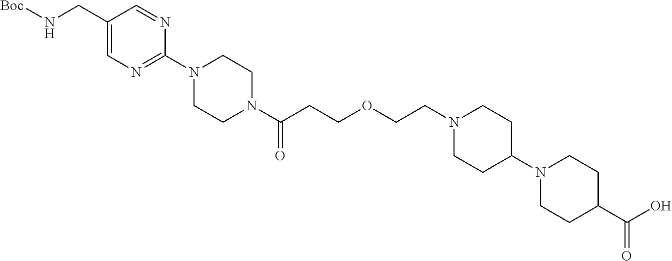 [SMILES: CC(C)(C)OC(=O)NCc1cnc(N2CCN(C(=O)CCOCCN3CCC(N4CCC(C(=O)O)CC4)CC3)CC2)nc1]

### Step 1: Synthesis of 1′-tert-butyl 4-ethyl [1,4′-bipiperidine]-1′,4-dicarboxylate

To a solution of ethyl piperidine-4-carboxy late (30 g, 150.57 mmol, 1.0 equiv) and tert-butyl 4-oxopiperidine-1-carboxylate (23.67 g, 150.57 mmol, 1.0 equiv) in DCM (300 mL) was added HOAc (6.00 mL, 104.95 mmol, 0.7 equiv). The mixture was stirred at room temperature for 30 min, then NaBH(OAc)₃ (63.82 g, 301.13 mmol, 2.0 equiv) was added. The mixture was stirred for 16 h, at which point H₂O (50 mL) was added. The aqueous phase was extracted with DCM (3×15 mL) and the combined organic phases were washed with brine (10 mL), dried, filtered, and concentrated under reduced pressure. Purification by silica gel chromatography (8→100 MeOH/EtOAc) afforded the desired product as a yellow oil (30 g, 59% yield).

### Step 2: Synthesis of ethyl [1,4′-bipiperidine]-4-carboxylate

To a solution of HCl in EtOAc (200 mL) was added 1′-tert-butyl 4-ethyl [1,4′-bipiperidine]-1′,4-dicarboxylate (20 g, 58.74 mmol, 1.0 equiv). The mixture was stirred at room temperature for 3 h. The mixture was then concentrated under reduced pressure to afford the desired crude product as a white solid (15 g, HCl salt).

### Step 3: Synthesis of ethyl 1′-(2-(3-(tert-butoxy)-3-oxopropoxy)ethyl)-[1,4′-bipiperidine]-4-carboxylate

To a solution of tert-butyl 3-(2-bromoethoxy)propanoate (6.46 g, 25.54 mmol, 1.0 equiv) in DMF (240 mL) was added K₂CO₃ (10.59 g, 76.61 mmol, 3.0 equiv) and ethyl [1,4′-bipiperidine]-4-carboxylate (8 g, 25.54 mmol, 1.0 equiv, 2HCl). The mixture was stirred at 120° C. for 12 h, at which point the reaction was cooled to room temperature, filtered, the filter cake washed with H₂O (20 mL), and the filtrate was concentrated under reduced pressure. Purification by silica gel chromatography (0→11% MeOH/EtOAc) afforded the desired product as a yellow oil (6.6 g, 63% yield).

### Step 4: Synthesis of 3-(2-(4-(ethoxycarbonyl)-[1,4′-bipiperidin]-1′-yl)ethoxy)propanoic acid

To the solution of HCl in EtOAc (70 mL) was added ethyl 1′-(2-(3-(tert-butoxy)-3-oxopropoxy) ethyl)-[1,4′-bipiperidine]-4-carboxylate (6.6 g, 16.00 mmol, 1.0 equiv). The mixture was stirred at room temperature for 3 h, at which point the reaction was concentrated under reduced pressure to afford the desired product as a white solid (6.5 g, 95% yield. 2HCl).

### Step 5: Synthesis of ethyl 1′-(2-(3-(4-(5-(((N,N-di-tert-butoxycarbonyl)amino)methyl)pyrimidin-2-yl)piperazin-1-yl)-3-oxopropoxy)ethyl)-[1,4′-bipiperidine]-4-carboxylate

To a solution of tert-butyl-tert-butoxycarbonyl((2-(piperazin-1-yl)pyrimidin-5-yl)methyl)carbamate (2.49 g, 6.33 mmol, 1.5 equiv) in DMF (40 mL) was added DIPEA (9.74 mL, 55.89 mmol, 6.0 equiv) and HATU (5.31 g, 13.97 mmol, 1.5 equiv). The mixture was stirred at room temperature for 30 min, and then 3-(2-(4-(ethoxycarbonyl)-[1,4′-bipiperidin]-1′-yl)ethoxy) propanoic acid (4 g, 9.32 mmol, 1.0 equiv. 2HCl) was added. The mixture was stirred at for 1.5 h, at which point H₂O (5 mL) and EtOAc (20 mL) were added. The aqueous phase was extracted with EtOAc (3×10 mL) and the combined organic phases were washed with brine (5 mL), dried, filtered and concentrated under reduced pressure. Purification by reverse phase chromatography afforded the desired product as a brown oil (1.6 g, 23% yield). LCMS (ESI) m/z: [M+H] calcd for C₃₇H₆₁N₇O₈: 732.47; found 732.6.

### Step 6: Synthesis of 1′-(2-(3-(4-(5-(((tert-butoxycarbonyl)amino)methyl)pyrimidin-2-yl)piperazin-1-yl)-3-oxopropoxy)ethyl)-[1,4′-bipiperidine]-4-carboxylic acid

To a solution of ethyl 1′-(2-(3-(4-(5-(((N,N-di-tert-butoxycarbonyl)amino)methyl)pyrimidin-2-yl)piperazin-1-yl)-3-oxopropoxy)ethyl)-[1,4′-bipiperidine]-4-carboxylate (1.4 g, 1.91 mmol, 1.0 equiv) in THF (7.5 mL), EtOH (3.8 mL), and H₂O (3.8 mL) was added LiOH·H₂O (321.07 mg, 7.65 mmol, 4.0 equiv). The mixture was stirred at room temperature for 2 h, at which point the mixture was concentrated under reduced pressure. Purification by reverse phase chromatography (5→38% MeCN/H₂O) afforded the desired product as a yellow solid (325 mg, 22% yield). LCMS (ESI) m/z: [M+H] calcd for C₃₀H₄₉N₇O₆: 604.38; found 604.3.

### Building Block AZ. 1-(4-{2-[2-(2-{[(benzyloxy)carbonyl]amino}ethoxy)ethoxy]ethyl}piperazin-1-yl)-3,6,9,12-tetraoxapentadecan-15-oic acid

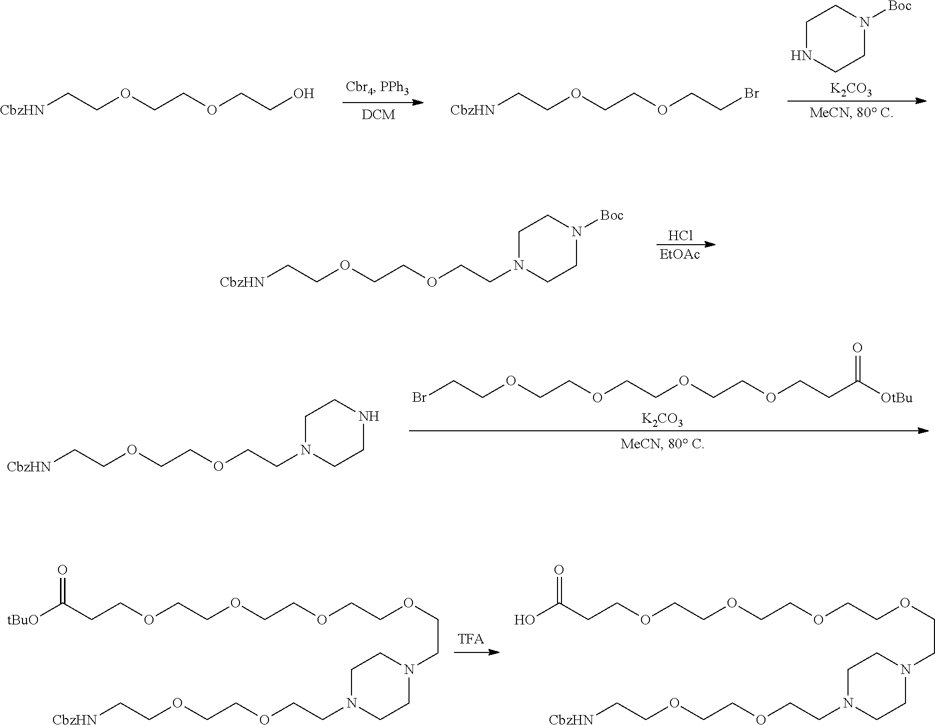

### Step 1: Synthesis of benzyl (2-(2-(2-bromoethoxy)ethoxy)ethyl)carbamate

To a solution of benzyl (2-(2-(2-hydroxyethoxy)ethoxy)ethyl)carbamate (10 g, 35.30 mmol, 1.0 equiv) in DCM (300 mL) at 0° C. was added PPh₃ (13.79 g, 52.59 mmol, 1.49 equiv) and CBr₄ (17.44 g, 52.59 mmol, 1.49 equiv). Then the mixture was warmed to room temperature and stirred for 12 h. The reaction mixture was then filtered, and the filtrate was concentrated under reduced pressure. Purification by silica gel chromatography (1%→25% EtOAc/petroleum ether) afforded the desired product (10.8 g, 88.4% yield) as yellow oil.

### Step 2: Synthesis of tert-butyl 4-(3-oxo-1-phenyl-2,7,10-trioxa-4-azadodecan-12-yl)piperazine-1-carboxylate

To a solution of benzyl (2-(2-(2-bromoethoxy)ethoxy)ethyl)carbamate (10.8 g, 31.19 mmol, 1.0 equiv) and tert-butyl piperazine-1-carboxylate (5.81 g, 31.19 mmol, 1.0 equiv) in MeCN (100 mL) was added K₂CO₃ (4.31 g, 31.19 mmol, 1.0 equiv). The mixture was stirred at 80° C. for 1 h. The reaction mixture was then filtered, and the filtrate was concentrated under reduced pressure. Purification by silica gel chromatography (0%→50% MeOH/EtOAc) afforded the desired product (13.1 g, 93.0% yield) as yellow oil.

### Step 3: Synthesis of benzyl (2-(2-(2-(piperazin-1-yl)ethoxy)ethoxy)ethyl)carbamate

A solution of tert-butyl 4-(3-oxo-1-phenyl-2,7,10-trioxa-4-azadodecan-12-yl)piperazine-1-carboxylate (5.64 g, 12.49 mmol, 1.0 equiv) in HCl/EtOAc (50 mL, 4 M) was stirred at room temperature for 1 h. The reaction mixture was then concentrated under reduced pressure to afford the desired product (5.23 g, crude. HCl salt) as yellow oil.

### Step 4: Synthesis of tert-butyl 1-(4-(3-oxo-1-phenyl-2,7,10-trioxa-4-azadodecan-12-yl)piperazin-1-yl)-3,6,9,12-tetraoxapentadecan-15-oate

A solution of benzyl (2-(2-(2-(piperazin-1-yl)ethoxy)ethoxy)ethyl)carbamate (13.3 g, 31.34 mmol, 1.0 equiv, 2HCl) and tert-butyl 1-bromo-3,6,9,12-tetraoxapentadecan-15-oate in MeCN (150 mL) was added K₂CO₃ (21.66 g, 156.71 mmol, 5.0 equiv). The mixture was stirred at 80° C. for 12 h. The reaction mixture was then filtered, and the filtrate was concentrated under reduced pressure. Purification by silica gel chromatography (1%→17% MeOH/DCM) afforded the desired product (5.4 g, 26.3% yield) as a yellow oil.

### Step 5: Synthesis of 1-(4-(3-oxo-1-phenyl-2,7,10-trioxa-4-azadodecan-12-yl)piperazin-1-yl)-3,6,9,12-tetraoxapentadecan-15-oic acid

A solution of tert-butyl 1-(4-(3-oxo-1-phenyl-2,7,10-trioxa-4-azadodecan-12-yl)piperazin-1-yl)-3,6,9,12-tetraoxapentadecan-15-oate (2.4 g, 3.66 mmol, 1.0 equiv) in TFA (20 mL) was stirred at room temperature for 30 min. The reaction mixture was then concentrated under reduced pressure to afford the desired product (3.03 g, TFA salt) as yellow oil.

Building Block BA. (R)-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylicacid

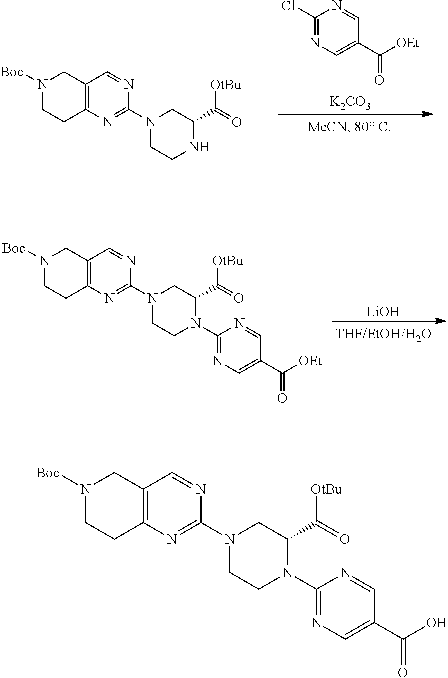 [SMILES: CC(C)(C)OC(=O)[C@H]1CN(c2ncc3c(n2)CCN(C(=O)OC(C)(C)C)C3)CCN1 ; "K2CO3" ; CCOC(=O)c1cnc(Cl)nc1 ; CCOC(=O)c1cnc(N2CCN(c3ncc4c(n3)CCN(C(=O)OC(C)(C)C)C4)C[C@@H]2C(=O)OC(C)(C)C)nc1 ; "LiOH" ; CC(C)(C)OC(=O)[C@H]1CN(c2ncc3c(n2)CCN(C(=O)OC(C)(C)C)C3)CCN1c1ncc(C(=O)O)cn1]

### Step 1: Synthesis of (R)-tert-butyl 2-(3-(tert-butoxycarbonyl)-4-(5-(ethoxycarbonyl)pyrimidin-2-yl)piperazin-1-yl)-7,8-dihydropyrido[4,3-d]pyrimidine-6(5H)-carboxylate

To two separate batches run in parallel each containing a solution of (R)-tert-butyl 2-(3-(tert-butoxycarbonyl)piperazin-1-yl)-7,8-dihydropyrido[4,3-d]pyrimidine-6(5H)-carboxylate (6 g, 14.30 mmol, 1.0 equiv) and K₂CO₃ (3.95 g, 28.60 mmol, 2.0 equiv) in MeCN (80 mL) was added ethyl 2-chloropyrimidine-5-carboxylate (3.20 g, 17.16 mmol, 1.2 equiv). The reaction mixtures were stirred at 80° C. for 12 h. The two reactions mixtures were combined and filtered, the residue was washed with EtOAc (3×50 mL), and the filtrate was concentrated under reduced pressure. Purification by silica gel chromatography (0%→17% MeOH/EtOAc) afforded the desired product (15 g, 91.5% yield) as a yellow solid. LCMS (ESI) m/z: [M+H] calcd for C₂₈H₃₉N₇O₆: 570.31; found 570.1.

### Step 2: Synthesis of (R)-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

To a solution of (R)-tert-butyl 2-(3-(tert-butoxycarbonyl)-4-(5-(ethoxycarbonyl)pyrimidin-2-yl)piperazin-1-yl)-7,8-dihydropyrido[4,3-d]pyrimidine-6(5H)-carboxylate (15 g, 26.33 mmol, 1.0 equiv) in THF (80 mL), EtOH (40 mL) and H₂O (40 mL) was added LiOH·H₂O (2.21 g, 52.66 mmol, 2.0 equiv). The mixture was stirred at room temperature for 6 h. The reaction mixture was then adjusted to pH=6 with 1 N HCl. The resulting suspension was filtered, and the solid cake was dried under reduced pressure to afford the desired product (10.87 g, 75.9% yield) as a white solid. LCMS (ESI) m/z: [M+H] calcd for C₂₆H₃₅N₇O₆: 542.27; found 542.1.

### Building Block BB. (S)-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

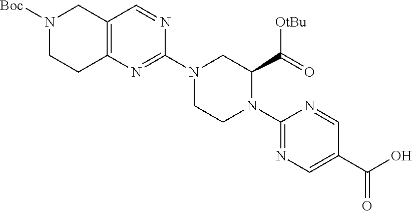 [SMILES: CC(C)(C)OC(=O)[C@@H]1CN(c2ncc3c(n2)CCN(C(=O)OC(C)(C)C)C3)CCN1c1ncc(C(=O)O)cn1]

This building block is prepared from Building block AA by a process similar to that for Building block BA.

### Building Block BC. 2-[(2R)-2-[(tert-butoxy)carbonyl]-4-[5-({[(tert-butoxy)carbonyl]amino}methyl)pyrimidin-2-yl]piperazin-1-yl]pyrimidine-5-carboxylic acid

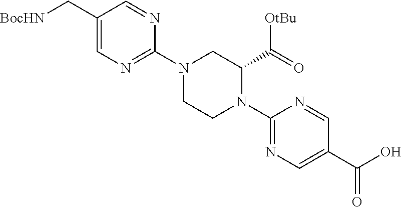 [SMILES: CC(C)(C)OC(=O)NCc1cnc(N2CCN(c3ncc(C(=O)O)cn3)[C@@H](C(=O)OC(C)(C)C)C2)nc1]

This building block is prepared from Building block AU by a process similar to that for Building block BA.

### Building Block BD. 2-[(2S)-2-[(tert-butoxy)carbonyl]-4-[5-({[(tert-butoxy)carbonyl]amino}methyl)pyrimidin-2-yl]piperazin-1-yl]pyrimidine-5-carboxylic acid

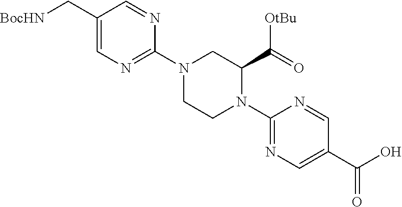 [SMILES: CC(C)(C)OC(=O)NCc1cnc(N2CCN(c3ncc(C(=O)O)cn3)[C@H](C(=O)OC(C)(C)C)C2)nc1]

This building block is prepared from Building block AV by a process similar to that for Building block BA.

### Building Block BE. 15-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-1-((1S,4S)-5-(2-(2-(2-aminoethoxy)ethoxy)ethyl)-2,5-diazabicyclo[2.2.1]heptan-2-yl)-3,6,9,12-tetraoxapentadecan-15-one

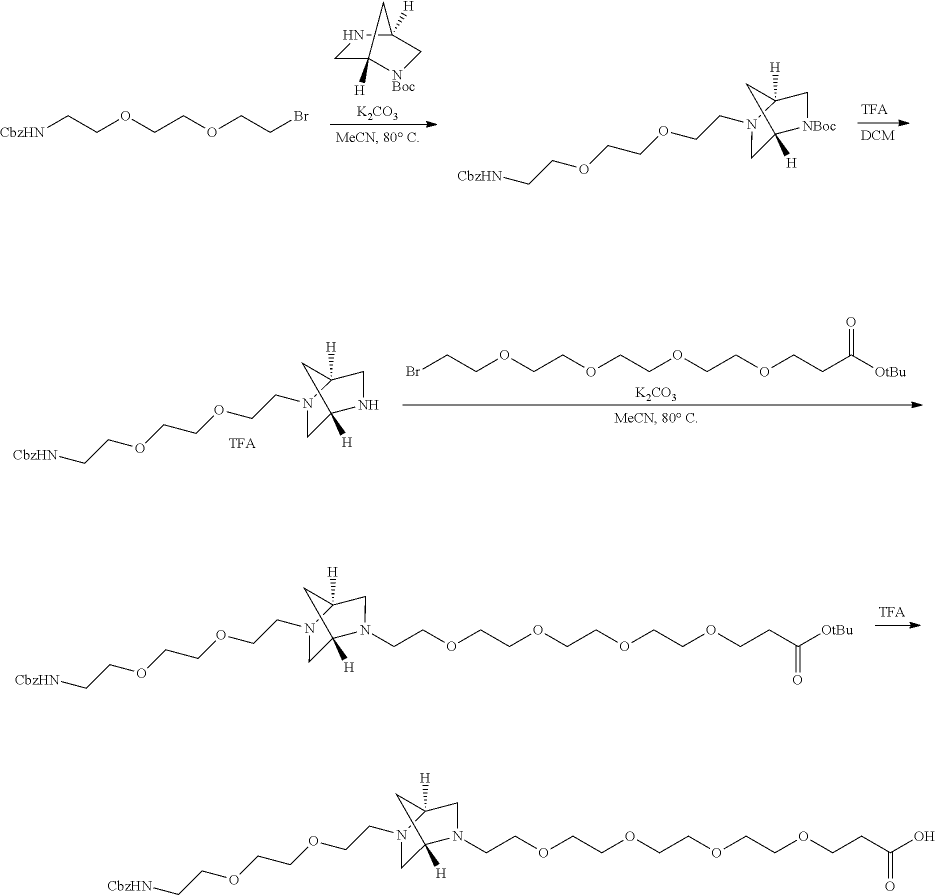

### Step 1: Synthesis of (1S,4S)-tert-butyl 5-(3-oxo-1-phenyl-2,7,10-trioxa-4-azadodecan-12-yl)-2,5-diazabicyclo[2.2.1]heptane-2-carboxylate

To a solution of (1S,4S)-tert-butyl 2,5-diazabicyclo[2.2.1]heptane-2-carboxylate (2.85 g, 14.37 mmol, 1.0 equiv) in MeCN (50 mL) was added K₂CO₃ (3.97 g, 28.75 mmol, 2.0 equiv) and benzyl (2-(2-(2-bromoethoxy)ethoxy)ethyl)carbamate (4.98 g, 14.37 mmol, 1.0 equiv). The mixture was stirred at 80° C. for 24 h. The reaction mixture was then filtered, and the filtrate was concentrated under reduced pressure. Purification by silica gel chromatography (0→10% MeOH/EtOAc) afforded the desired product (6.2 g, 93.0% yield) as colorless oil. LCMS (ESI) m/z: [M+H] calcd for C₂₄H₃₇N₃O₆: 464.27; found 464.2.

### Step 2: Synthesis of benzyl (2-(2-(2-((1S,4S)-2,5-diazabicyclo[2.2.1]heptan-2-yl)ethoxy)ethoxy)ethyl)carbamate

To a solution of (1S,4S)-tert-butyl 5-(3-oxo-1-phenyl-2,7,10-trioxa-4-azadodecan-12-yl)-2,5-diazabicyclo[2.2.1]heptane-2-carboxylate (6.2 g, 13.37 mmol, 1.0 equiv) in DCM (60 mL) was added TFA (20.7 mL, 279.12 mmol, 20.9 equiv). The reaction was stirred for 2 h, at which point the mixture was concentrated under reduced pressure at 45° C. to afford the desired crude product (10.5 g, 4TFA) as light brown oil, which was used the next step directly. LCMS (ESI) m/z: [M+H] calcd for C₁₉H₂₉N₃O₄: 364.22; found 364.2.

### Step 3: Synthesis of tert-butyl 1-((1S,4S)-5-(3-oxo-1-phenyl-2,7,10-trioxa-4-azadodecan-12-yl)-2,5-diazabicyclo[2.2.1]heptan-2-yl)-3,6,9,12-tetraoxapentadecan-15-oate

To a solution of benzyl (2-(2-(2-((1S,4S)-2,5-diazabicyclo[2.2.1]heptan-2-yl)ethoxy) ethoxy)ethyl)carbamate (5 g, 6.10 mmol, 1.0 equiv, 4TFA) in MeCN (80 mL) was added K₂CO₃ (5.06 g, 36.61 mmol, 6.0 equiv) and tert-butyl 1-bromo-3,6,9,12-tetraoxapentadecan-15-oate (2.35 g, 6.10 mmol, 1.0 equiv). The reaction mixture was stirred at 80° C. for 12 h. The reaction mixture was then filtered, and the filtrate was concentrated under reduced pressure. Purification by silica gel chromatography (0→15% MeOH/EtOAc) afforded the desired product (5.2 g, 92.8% yield) as light yellow oil. LCMS (ESI) m/z: [M+H] calcd for C₃₄H₅₇N₃O₁₀: 668.4; found 668.4.

### Step 4: Synthesis of 1-((1S,4S)-5-(3-oxo-1-phenyl-2,7,10-trioxa-4-azadodecan-12-yl)-2,5-diazabicyclo[2.2.1]heptan-2-yl)-3,6,9,12-tetraoxapentadecan-15-oic acid

A solution of tert-butyl 1-((1S,4S)-5-(3-oxo-1-phenyl-2,7,10-trioxa-4-azadodecan-12-yl)-2,5-diazabicyclo[2.2.1]heptan-2-yl)-3,6,9,12-tetraoxapentadecan-15-oate (5.2 g, 5.66 mmol, 1.0 equiv) in TFA (47.3 mL, 638.27 mmol, 112.75 equiv) was stirred at room temperature for 30 min. The mixture was then concentrated under reduced pressure at 45° C. Purification by reverse phase chromatography (2→35% MeCN/H₂O (0.05% NH₄OH)) afforded the desired product (1.88 g, 54.3% yield) as light brown oil. LCMS (ESI) m/z: [M+H] calcd for C₃₀H₄₉N₃O₁₀: 612.34; found 612.3.

### Building Block BF. 21-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-1-(piperazin-1-yl)-3,6,9,12,15,18-hexaoxahenicosan-21-one

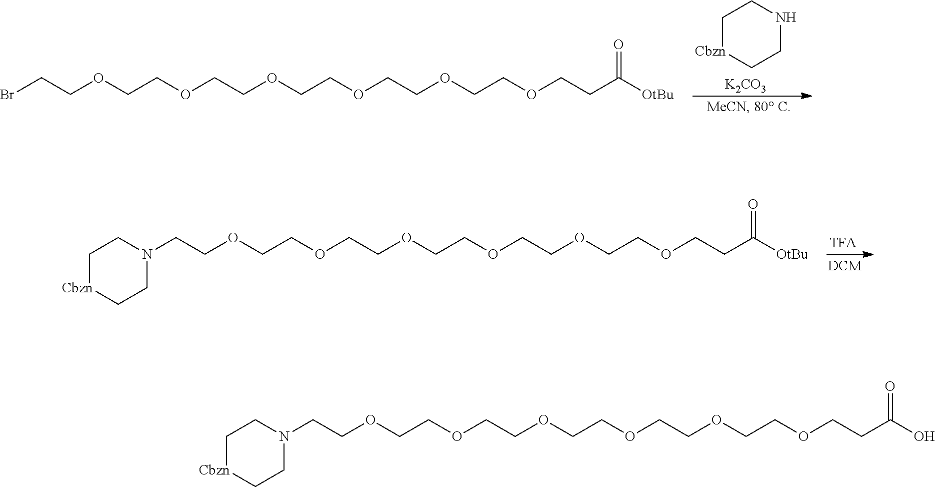

### Step 1: Synthesis of benzyl 4-(23,23-dimethyl-21-oxo-3,6,9,12,15,18,22-heptaoxatetracosyl) piperazine-1-carboxylate

To a solution of tert-butyl 1-bromo-3,6,9,12,15,18-hexaoxahenicosan-21-oate (5 g, 10.56 mmol, 1.0 equiv) and benzyl piperazine-1-carboxylate (2.62 mL, 11.62 mmol, 1.1 equiv, HCl) in MeCN (50 mL) was added K₂CO₃ (4.38 g, 31.69 mmol, 3.0 equiv). The reaction mixture was stirred at 80° C. for 10 h. The mixture was then filtered, the solid cake washed with EtOAc (3×3 mL), and the filtrate concentrated under reduced pressure. Purification by silica gel chromatography (0→10% MeOH/EtOAc) afforded the desired product (4 g, 61.8% yield) as a red liquid.

### Step 2: Synthesis of 1-(4-((benzyloxy)carbonyl)piperazin-1-yl)-3,6,9,12,15,18-hexaoxahenicosan-1-oic acid

To a solution of benzyl 4-(23,23-dimethyl-21-oxo-3,6,9,12,15,18,22-heptaoxatetracosyl)piperazine-1-carboxylate (1.8 g, 2.94 mmol, 1.0 equiv) in DCM (10 mL) was added TFA (10 mL). The solution was stirred for 0.5 h. The solution was then concentrated under reduced pressure. To the residue was added DCM (30 mL) and then the solution was concentrated under reduced pressure to afford the desired product (1.6 g, 2.87 mmol, TFA) as a red liquid.

### Preparation of Rapamycin Monomers.

### Monomer 1. 40(R)—O-(4-nitrophenyl)carbonate rapamycin

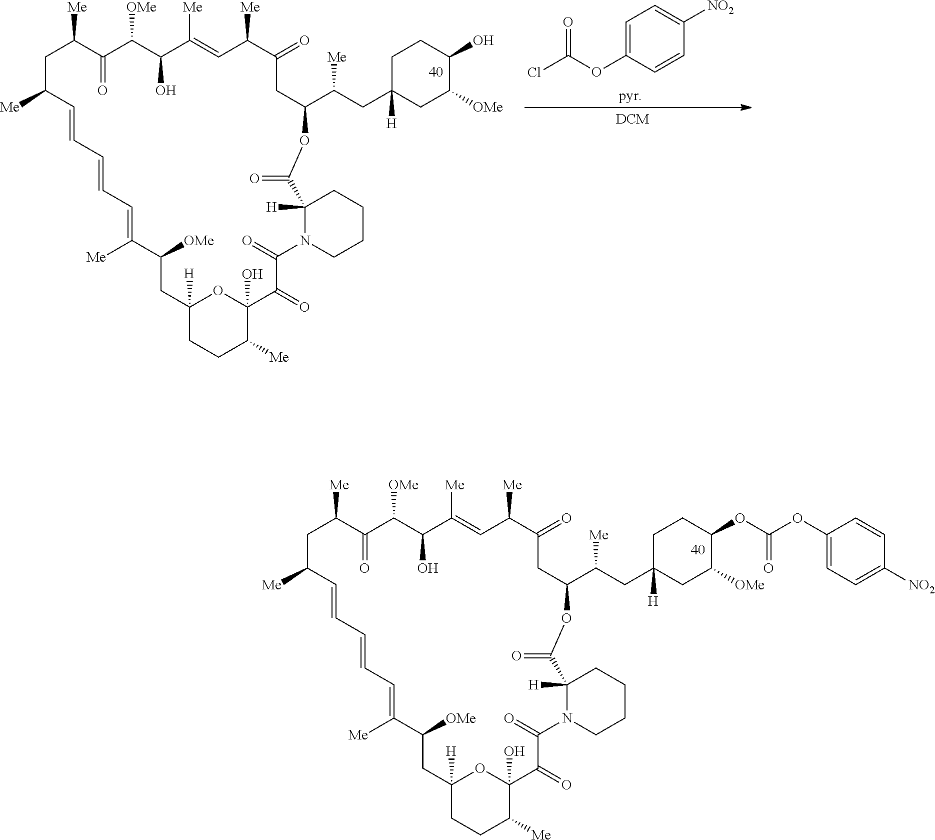 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C ; O=C(Cl)Oc1ccc([N+](=O)[O-])cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C]

To a solution of rapamycin (30.10 g, 32.92 mmol, 1.0 equiv) in DCM (148.9 mL) was added pyridine (29.6 mL, 367 mmol, 11.1 equiv). The solution was cooled to −78° C. and then p-nitrophenyl chloroformate (12.48 g, 61.92 mmol, 1.9 equiv) was added. The reaction was stirred at −78° C. for 2 h. To the reaction mixture was then added DCM and the solution was then poured into H₂O. The aqueous layer was extracted with DCM and the combined organic layers were dried over MgSO₄, and concentrated under reduced pressure. The crude material was purified by silica gel chromatography (0→50% EtOAc/hexanes) to provide the product (23.1 g, 59.2% yield) as a white solid. LCMS (ESI) m/z: [M+Na] calcd for C₅₈H₈₂N₂O₁₇: 1101.55; found 1101.6.

### Monomer 2. 32(R)-hydroxy 40(R)—O-(4-nitrophenyl)carbonate rapamycin

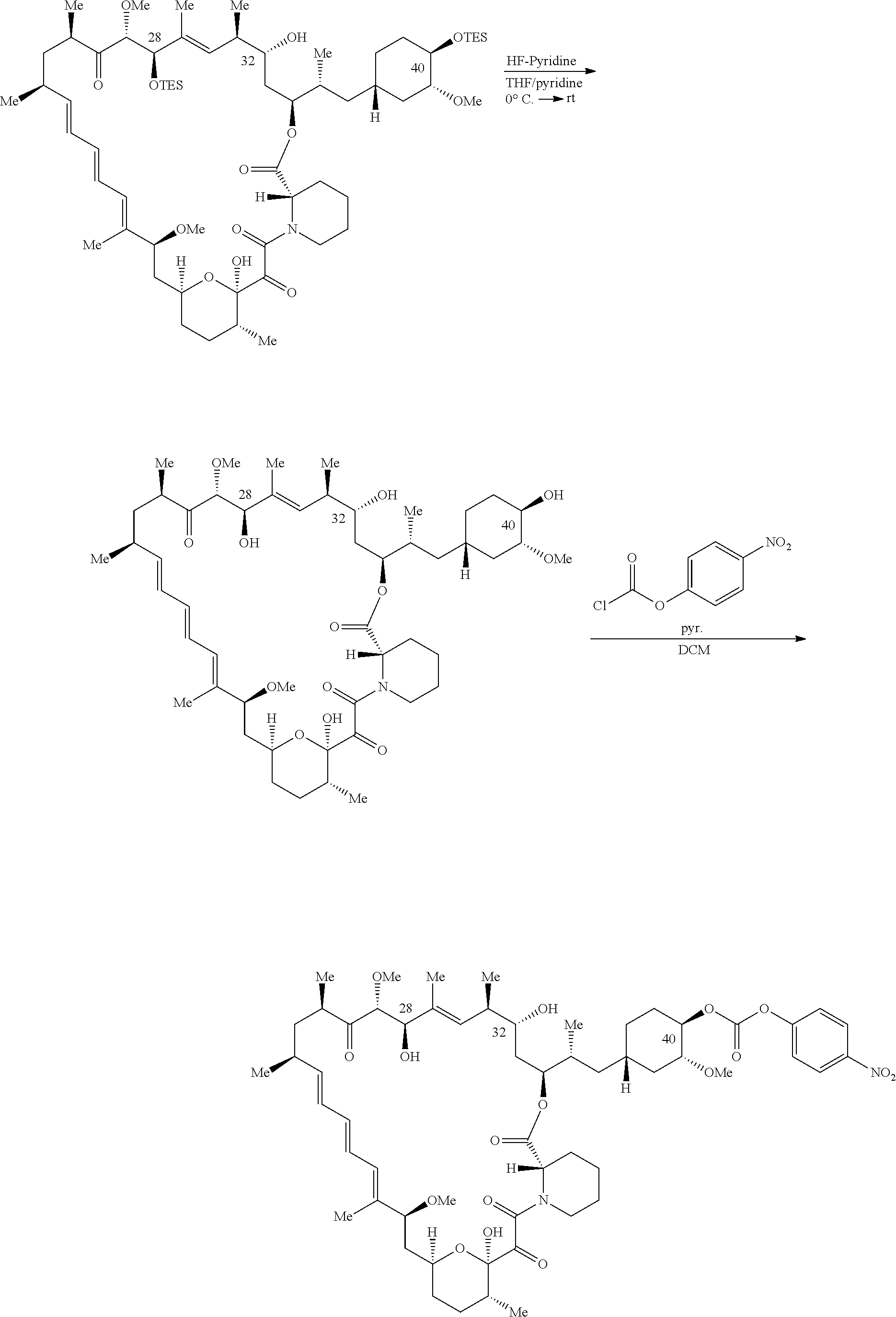

### Step 1: Synthesis of 32(R)-hydroxy rapamycin

A solution of 32(R)-hydroxy-28,40-bistriethylsilyl rapamycin (3.64 g, 3.18 mmol, 1 equiv) in THF (41.8 mL) was treated with pyridine (20.8 mL, 258 mmol, 81 equiv) and the reaction mixture was cooled to 0° C. The solution was treated dropwise with 70% HF-pyridine (4.60 mL, 159 mmol, 50 equiv) and the reaction mixture was stirred at 0° C. for 20 min followed by warming to room temperature. After 5 h, the reaction mixture was cooled back to 0° C. and carefully added to ice cold sat. NaHCO₃ solution (400 mL). The mixture was extracted with EtOAc (2×100 mL) and the organic phases were washed with 75 mL portions of H₂O, sat. NaHCO₃ solution and brine. The organic solution was dried over Na₂SO₄, filtered and concentrated to yield a light yellow oil that produced a stiff foam under reduced pressure. The crude material was purified by silica gel chromatography (20→40% acetone/hexanes) to yield the desired product as a white amorphous solid (1.66 g, 57% yield). LCMS (ESI) m/z: [M+Na] calcd for C₅₁H₈₁NO₁₃: 938.56; found 938.7; m/z: [M−H] calcd for C₅₁H₈₁NO₁₃: 914.56; found 914.7.

### Step 2: Synthesis of 32(R)-hydroxy 40(R)—O-(4-nitrophenyl)carbonate rapamycin

To a suspension of powdered 4 Å molecular sieves (6.0 g) in DCM (130 mL) was added 32(R)-hydroxy rapamycin (6.00 g, 6.55 mmol, 1.0 equiv). After stirring at room temperature for 45 min, pyridine (5.99 mL, 74.0 mmol, 11.3 equiv) was added. The suspension was cooled to −15° C. and then 4-nitrophenylchloroformate (1.78 g, 8.84 mmol, 1.4 equiv) was then added. The reaction mixture was stirred at −10° C. for 2 h and then filtered, and the filter pad washed with DCM (140 mL). The filtrate was washed with sat. NaHCO₃ (130 mL). H₂O (130 mL) and brine (130 mL), dried over Na₂SO₄, filtered and concentrated under reduced pressure. The crude material was purified by silica gel chromatography (20→50% EtOAc/hexanes) to give the product (4.44 g, 63% yield) as an off-white stiff foam. LCMS (ESI) m/z: [M+Na] calcd for C₅₈H₈₄N₂O₁₇: 1103.57; found 1103.5.

### Monomer 3. 32(R)-methoxy 40(R)—O-(4-nitrophenyl)carbonate rapamycin

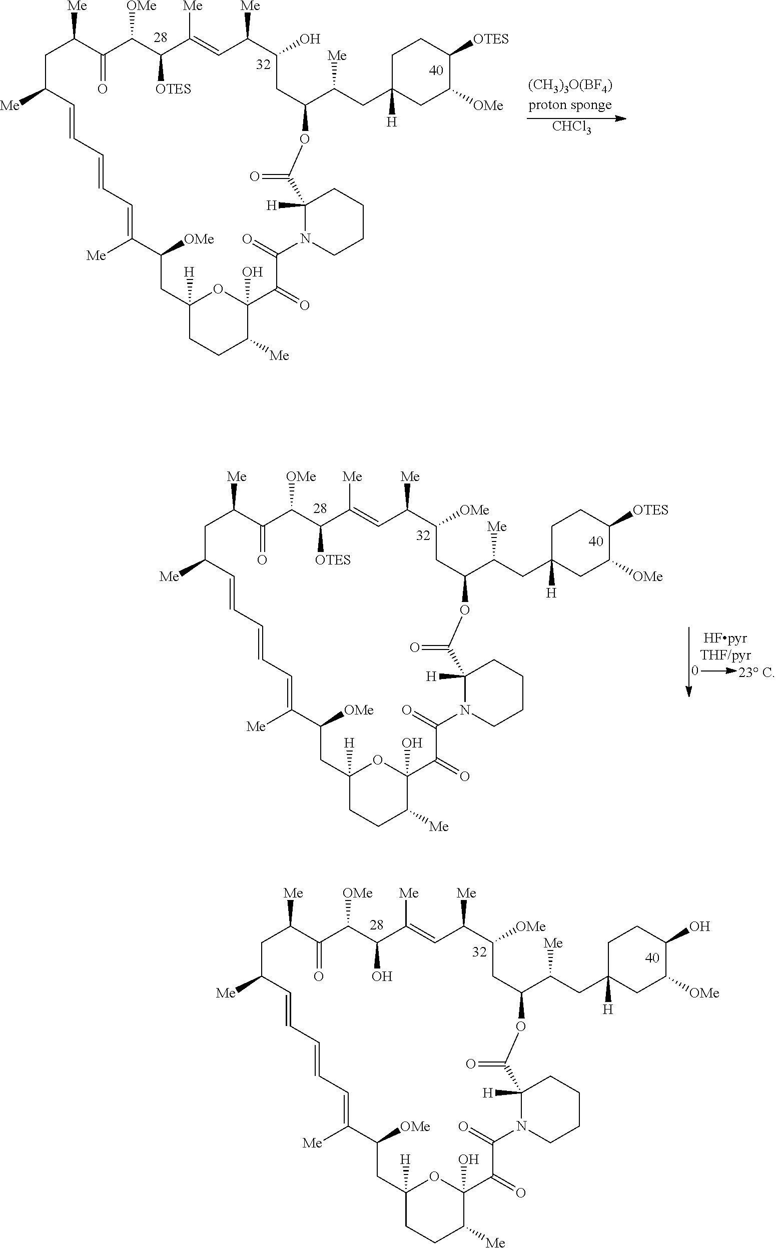

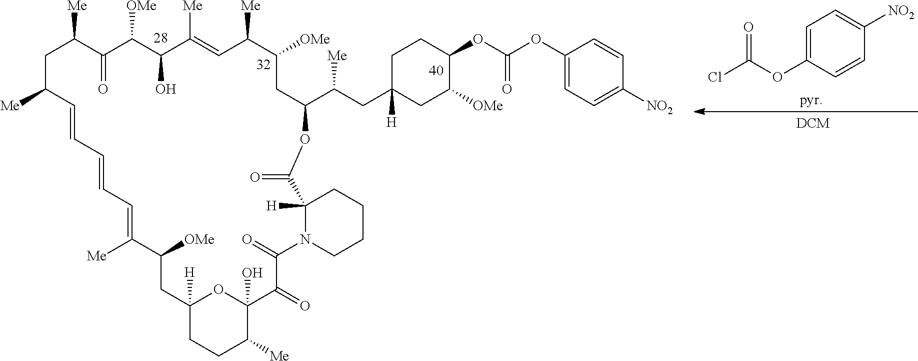 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C ; O=C(Cl)Oc1ccc([N+](=O)[O-])cc1]

### Step 1: Synthesis of 32(R)-methoxy-28,40-bistriethylsilyl rapamycin

To a stirred solution of 32(R)-hydroxy-28,40-bistriethylsilyl rapamycin (3.83 g, 3.34 mmol, 1.0 equiv) in chloroform (95.8 mL) was added Proton Sponge® (7.17 g, 33.5 mmol, 10.0 equiv) along with freshly dried 4 Å molecular sieves (4 g). The solution was stirred for 1 h prior to the addition of trimethyloxonium tetrafluoroborate (4.95 g, 33.5 mmol, 10.0 equiv, dried by heating under reduced pressure at 50° C. for 1 h before use) at room temperature. The reaction mixture was stirred for 18 h, and then the reaction mixture was diluted with DCM and filtered through Celite. The filtrate was washed sequentially with aqueous 1 M HCl (2×), sat. aqueous NaHCO₃ solution, then dried and concentrated under reduced pressure. Purification by silica gel chromatography (10→20% EtOAc/hexanes) afforded the desired product as a yellow oil that was contaminated with 3 wt. % Proton Sponge®. The residue was taken up in MTBE and washed with aqueous 1 M HCl, sat. aqueous NaHCO₃ solution, dried, and then concentrated under reduced pressure to furnish a yellow foam (3.15 g, 81.2% yield). LCMS (ESI) m/z: [M−TES+H₂O] calcd for C₆₄H₁₁₁NO₁₃Si₂: 1061.68; found 1061.9.

### Step 2: Synthesis of 32(R)-methoxy rapamycin

To a stirred solution of 32(R)-methoxy-28,40-bistriethylsilyl rapamycin (1.11 g, 0.958 mmol, 1.0 equiv) in THF (12.6 mL) and pyridine (6.30 mL) in a plastic vial was added 70% HF-pyridine (2.22 mL, 76.6 mmol, 80.0 equiv) dropwise at 0° C. The reaction mixture was stirred at 0° C. for 20 min before being warmed to room temperature for 3 h, when HPLC showed complete consumption of starting material. The reaction mixture was cooled to 0° C. and poured slowly into ice cold sat. aqueous NaHCO₃ solution (50 mL). The aqueous layer was extracted with EtOAc (3×) and the combined organics were washed with sat. aqueous NaHCO₃ solution, brine, dried, and concentrated under reduced pressure. The yellow residue was dissolved in MeOH (5 mL) and added dropwise to H₂O (50 mL) to produce a white precipitate. After stirring for 15 min the slurry was filtered on a medium porosity funnel and the cake washed with H₂O (2×). The solids were then dissolved in MeCN (50 mL) and lyophilized overnight to provide the product as a white solid (780 mg, 87% yield). LCMS (ESI) m/z: [M+Na] calcd for C₅₂H₈₃NO₁₃: 952.58; found 952.4.

### Step 3: Synthesis of 32(R)-methoxy 40(R)—O-(4-nitrophenyl)carbonate rapamycin

To a solution of 32(R)-methoxy rapamycin (4.50 g, 4.84 mmol, 1.0 equiv) in DCM (180 mL) was added powdered 4 Å molecular sieves (6.0 g). The mixture was stirred at room temperature for 1 h and then pyridine (3.91 mL, 48.4 mmol, 10 equiv) was added. The mixture was cooled to −10° C. and 4-nitrophenylchloroformate (0.990 g, 4.91 mmol, 1.0 equiv) was added in one portion. The reaction was allowed to slowly warm to room temperature and after 3 h the reaction mixture was cooled to 0° C. and 4-nitrophenylchloroformate (250 mg, 1.24 mmol, 0.3 equiv) was added. The mixture was warmed to room temperature and after 1 h the reaction mixture was filtered through a pad of celite and the pad was washed with DCM (140 mL). The filtrate was washed with H₂O (120 mL) and sat NaHCO₃ (2×120 mL). The organic phase was dried over Na₂SO₄, filtered and concentrated under reduced pressure. The crude material was purified by flash chromatography (20→50% EtOAc/hex) to yield a white stiff foam. The material was taken up in MeCN during which time a white solid formed. The solid was filtered, washed with additional MeCN and allowed to air dry to provide the product (4.51 g, 85% yield). LCMS (ESI) m/z [M+Na] calcd for C₅₉H₈₆N₂O₁₇: 1117.58; found 1117.6.

### Monomers 4. 32(R)-ethoxy 40(R)—O-(4-nitrophenyl)carbonate rapamycin

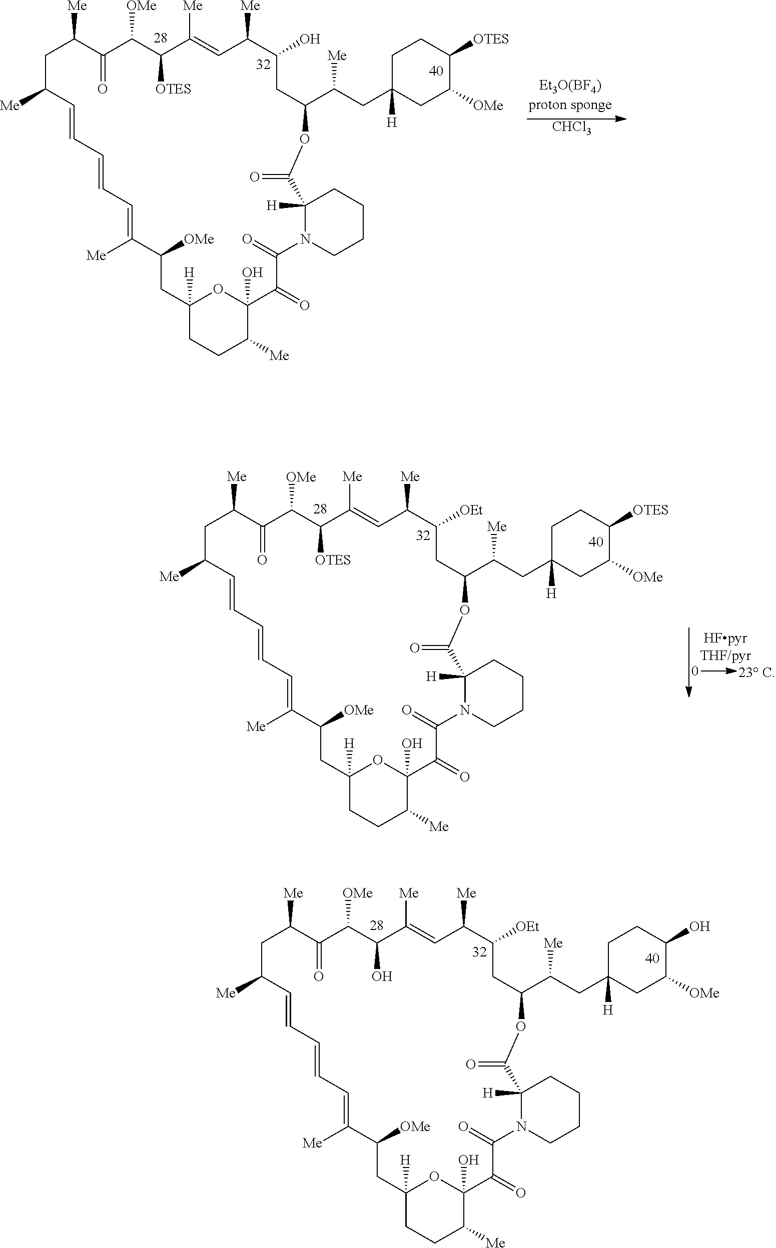

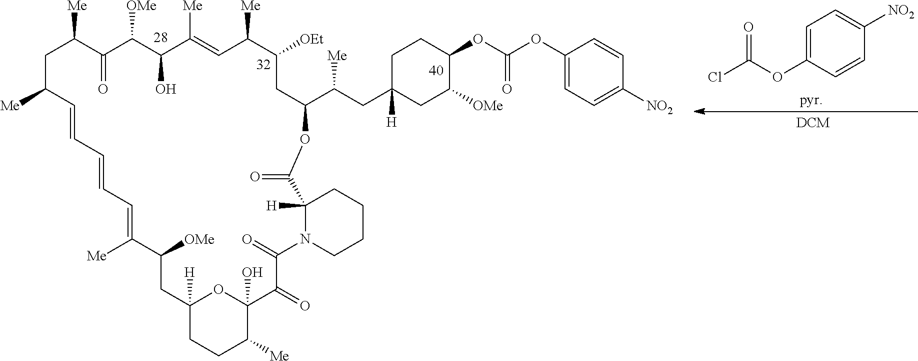 [SMILES: CCO[C@@H]1C[C@@H]([C@H](C)C[C@@H]2CC[C@@H](OC(=O)Oc3ccc([N+](=O)[O-])cc3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C ; O=C(Cl)Oc1ccc([N+](=O)[O-])cc1]

### Step 1: Synthesis of 32(R)-ethoxy-28,40-bistriethylsilyl rapamycin

A solution of 32(R)-hydroxy-28,40-bistriethylsilyl rapamycin (773 mg, 0.675 mmol, 1.0 equiv) in chloroform (19 mL) was treated with N,N,N′,N′-tetramethyl-1,8-naphthalenediamine (1.85 g, 8.63 mmol, 12.8 equiv) along with freshly dried 4 Å molecular sieves. The mixture was stirred for 1 h at room temperature and treated with triethyloxonium tetrafluoroborate (1.51 g, 7.95 mmol, 11.8 equiv) in one portion at room temperature. The reaction mixture was stirred for 3 h, at which point the reaction mixture was diluted with DCM and filtered through Celite, washing the filter pad with additional DCM. The combined filtrates were washed twice with 1 M HCl, once with saturated NaHCO₃ solution, and dried over Na₂SO₄. The solution was filtered and concentrated to a residue. The crude residue was treated with MTBE and filtered to remove polar insoluble material. The filtrate was concentrated and purified by silica gel chromatography (5→25% EtOAc/hex) to afford the product as a foam (516 mg, 65% yield). LCMS (ESI) m/z: [M+Na] calcd for C₆₅H₁₁₃NO₁₃Si₂ 1194.77; found 1194.6.

### Step 2: Synthesis of 32(R)-ethoxy rapamycin

To a solution of 32(R)-ethoxyethoxy-28,40-bistriethylsilyl rapamycin (131 mg. 0.112 mmol, 1.0 equiv) in THF (1.3 mL) at 0° C. was added pyridine (271 μL, 3.35 mmol, 3.4 equiv) followed by 70% HF-pyridine (51 μL, 1.8 mmol, 1.8 equiv). The reaction flask was capped and stored in the fridge for 3 days, at which point the reaction mixture was poured into cold saturated NaHCO₃ (20 mL). The aqueous layer extracted with EtOAc (3×20 mL) and the combined organic layers were washed with 1 M HCl (2×20 mL), saturated NaHCO₃ solution (20 mL), and brine. The solution was dried over Na₂SO₄, filtered, and concentrated. The residue was taken up in MeOH (1.5 mL) and added dropwise to H₂O (20 mL). The solids were filtered and washed with additional H₂O to provide the product (53 mg, 51% yield) as a white powder. LCMS (ESI) m/z: [M+Na] calcd for C₅₃H₈₅NO₁₃: 966.59; found 966.5.

### Step 3: Synthesis of 32(R)-ethoxy 40(R)—O-(4-nitrophenyl)carbonate rapamycin

To a 0.03 M solution of 32(R)-ethoxy rapamycin (1.0 equiv) in DCM is added powdered 4 Å molecular sieves. The mixture is stirred at room temperature for 1 h and then pyridine (10 equiv) is added. The mixture is cooled to −10° C. and 4-nitrophenylchloroformate (1.0 equiv) is added in one portion. The reaction is warmed to room temperature and stirred until consumption of 32(R)-ethoxy rapamycin, as determined by LCMS analysis. The mixture is filtered through a pad of celite and the pad washed with DCM. The filtrate is washed with H₂O and sat NaHCO₃. The organic phase is then dried over Na₂SO₄, filtered and concentrated under reduced pressure. The crude material is purified by flash chromatography (20→50% EtOAc/hex) to provide the product.

### Monomer 5. 40(R)—O-(4-nitrophenyl)thiocarbonate rapamycin

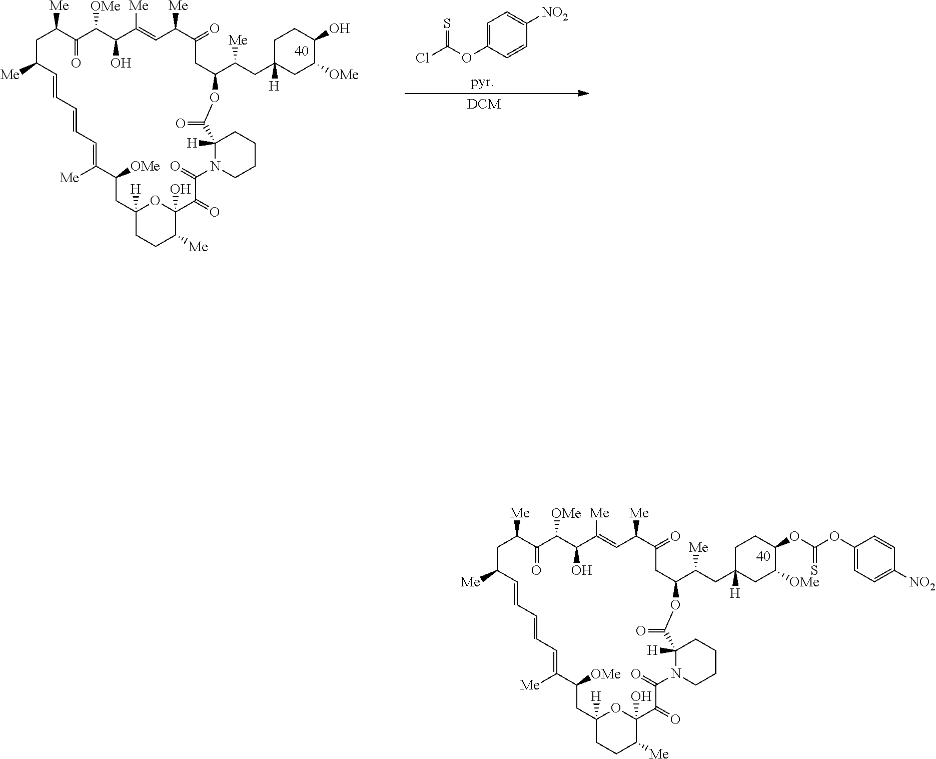 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C ; O=[N+]([O-])c1ccc(OC(=S)Cl)cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C]

To a solution of rapamycin (4.00 g, 4.38 mmol, 1.0 equiv) in DCM (20 mL) at −78° C. was added pyridine (4.0 mL, 49 mmol, 11.2 equiv), followed by a solution of O-(4-nitrophenyl)chlorothiocarbonate (1.19 g, 5.47 mmol, 1.3 equiv) in DCM (8.0 mL). The reaction mixture was warmed to −20° C. and stirred for 48 h. Hexane (40 mL) was then added and the resulting suspension was purified by silica gel chromatography (15/25/60 EtOAc/DCM/hexane then 20/25/55 EtOAc/DCM/hexane) to provide the product (3.09 g, 64.4% yield) as an off-white solid. LCMS (ESI) m/z: [M+Na] calcd for C₅₈H₈₂N₂O₁₆S: 1117.53; found 1117.5.

### Monomer 6. 28-O-(4-nitrophenyl)carbonate rapamycin

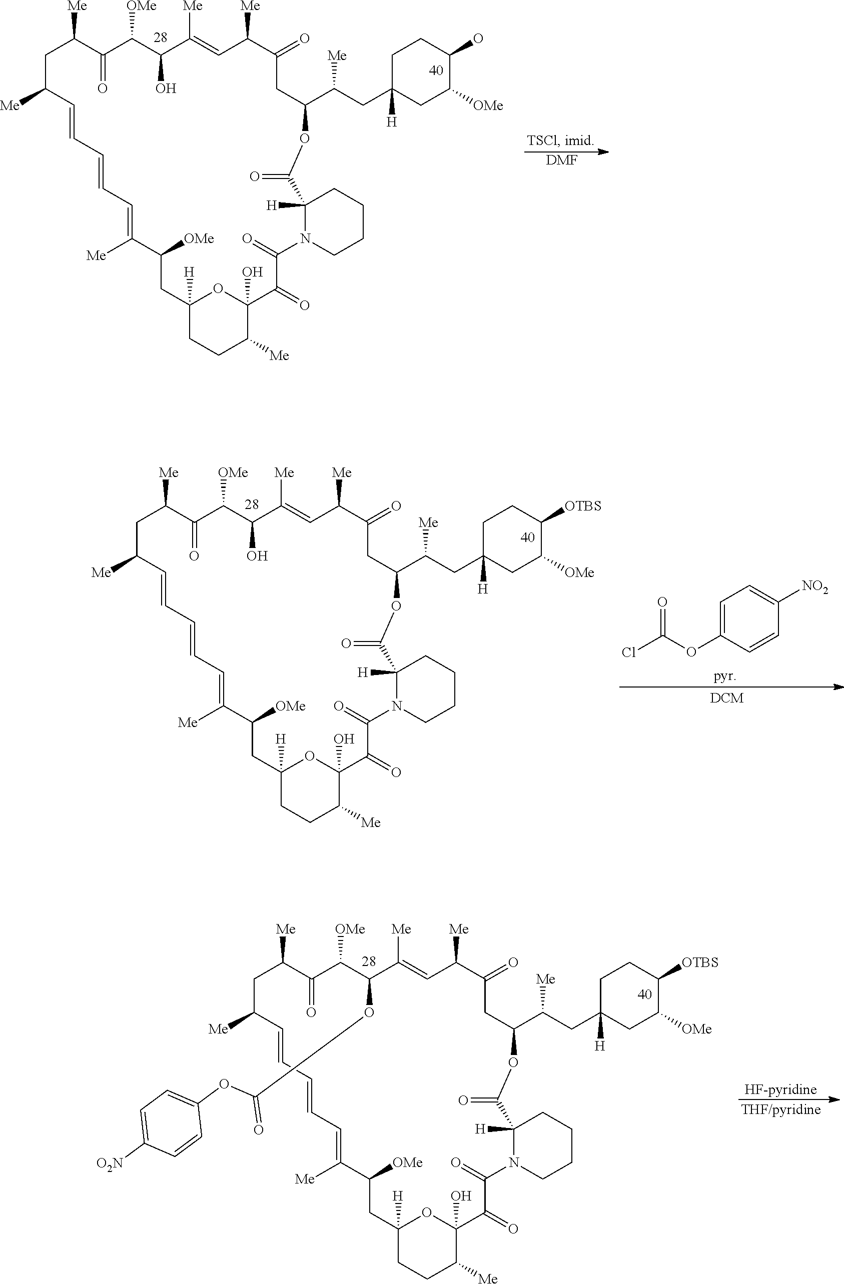

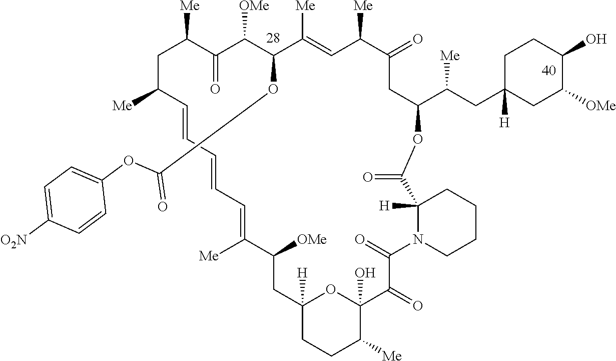 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)Oc2ccc([N+](=O)[O-])cc2)[C@@H](OC)C(=O)[C@H](C)C[C@H](C)/C=C/C=C/C=C/1C]

### Step 1: Synthesis of 40-O-tert-butyldimethylsilyl rapamycin

To a solution of rapamycin (1.00 g, 1.09 mmol, 1.0 equiv) in DMF (4 mL) at room temperature was added imidazole (0.22 g, 3.2 mmol, 2.9 equiv) followed by tert-butyldimethylsilyl chloride (0.176 g, 1.17 mmol, 1.07 equiv). The reaction mixture was stirred for 18 h. The reaction mixture was then diluted with DCM (100 mL) and washed with 20% aq. LiCl (3×20 mL). The organic layer was dried over Na₂SO₄, filtered and concentrated under reduced pressure. The residue was purified by silica gel chromatography (20→40% EtOAc/hexanes) to give the product (950 mg, 75% yield) as a faint yellow glass. LCMS (ESI) m/z: [M+H₂O] calcd for C₅₇H₉₃NO₁₃Si: 1045.65; found 1045.9.

### Step 2: Synthesis of 28-O-(4-nitrophenoxycarbonyl)-40-O-(tert-butyldimethylsilyl) rapamycin

To a solution of 40-O-tert-butyldimethylsilyl rapamycin (0.845 g, 0.822 mmol, 1.0 equiv) in DCM (10 mL) at room temperature was added pyridine (0.9 mL, 10 mmol, 12.1 equiv) followed by 4-nitrophenyl chloroformate (0.373 g, 1.85 mmol, 2.25 equiv). The reaction mixture was stirred for 2 h. The reaction mixture was then diluted with DCM (150 mL) and the solution sequentially washed with sat. NaHCO₃ (20 mL). 10% citric acid (2×20 mL), and brine (20 mL). The organic layer was dried over Na₂SO₄, filtered, and concentrated under reduced pressure. The residue was purified by reverse phase chromatography (30→100% MeCN/H₂O) to give the product (930 mg, 95% yield) as a pale yellow foam. LCMS (ESI) m/z: [M+H] calcd for C₆₄H₉₆N₂O₁₇Si: 1193.66; found 1193.7.

### Step 3: Synthesis of 28-O-(4-nitrophenoxycarbonyl) rapamycin

To a solution of 28-O-(4-nitrophenoxycarbonyl)-40-O-(tert-butyldimethylsilyl)rapamycin (0.930 g, 0.779 mmol, 1.0 equiv) in THF (10.7 mL) was added pyridine (3.78 mL, 46.8 mmol, 60.1 equiv) followed by the dropwise addition of 70% HF-pyridine (0.91 mL, 31.2 mmol, 40.0 equiv). The reaction mixture was stirred at room temperature for 48 h. The mixture was then poured slowly into ice cold sat. aqueous NaHCO₃ (20 mL). The aqueous layer was extracted with EtOAc (3×20 mL) and the combined organic layer was washed with sat. NaHCO₃ (10 mL) and brine (10 mL), dried over Na₂SO₄, filtered, and concentrated under reduced pressure. The residue was purified by reverse phase chromatography (30→100% MeCN/H₂O) to give the product (200 mg, 24% yield) as a faint yellow powder. LCMS (ESI) m/z: [M+Na] calcd for C₅₈H₈₂N₂O₁₇: 1101.55; found 1101.3.

### Monomer 7. 32(R)-hydroxy 40(R)—O-(4-nitrophenyl)thiocarbonate rapamycin

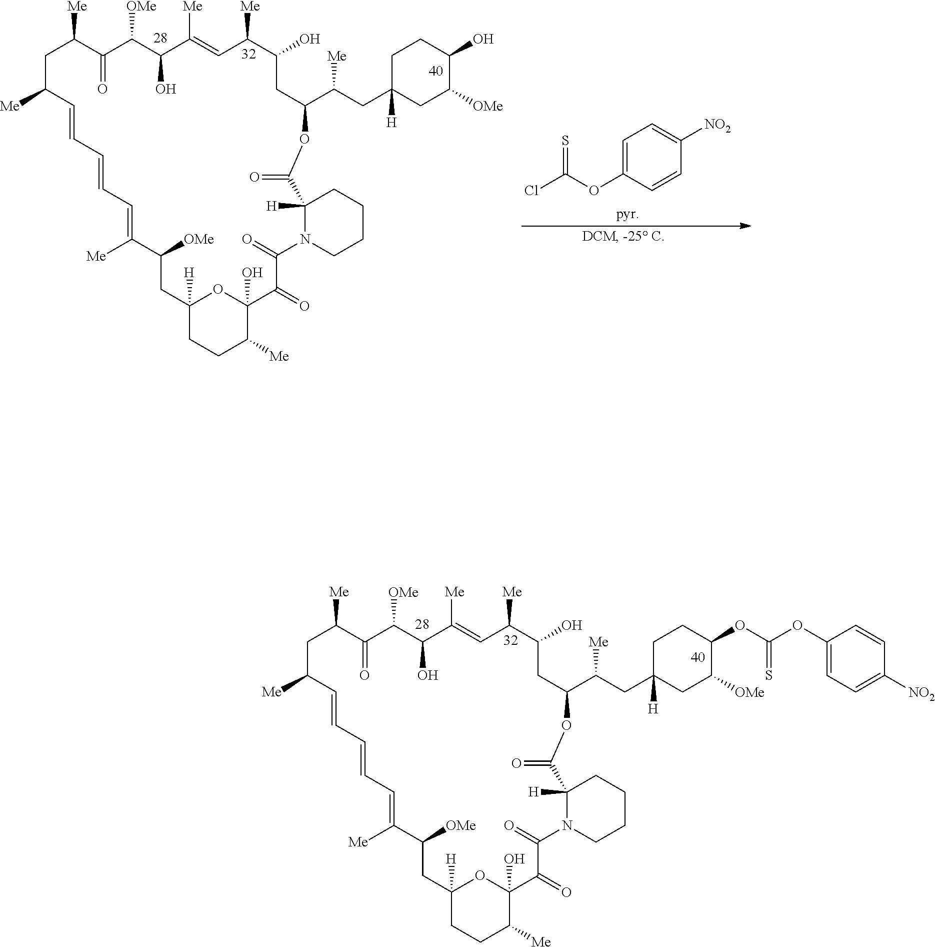 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; O=[N+]([O-])c1ccc(OC(=S)Cl)cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

To a solution of 32(R)-hydroxy rapamycin (2.80 g, 3.06 mmol, 1.0 equiv) in DCM (28 mL) was added pyridine (27.6 mL, 34 mmol, 11 equiv) and dried 4 Å molecular sieves (2.8 g). The suspension was stirred at room temperature for 1 h, at which point the mixture was cooled to −25° C. and a solution of O-(4-nitrophenyl)chlorothioformate (0.798 g, 3.67 mmol, 1.2 equiv) in DCM (6 mL) was added. The reaction was warmed to room temperature and after 21 h was filtered through Celite. The filtrate was partitioned between DCM and H₂O and the aqueous layer was extracted with DCM. The combined organic layers were dried and concentrated under reduced pressure. Purification by silica gel chromatography (EtOAc/hexanes) afforded the desired product as a white solid (2.15 g, 64% yield). LCMS (ESI) m/z: [M+Na] calcd for C₅₈H₈₄N₂O₁₆S: 1119.54; found 1120.0.

### Monomer 8. 32(R)-methoxy 40(R)—O-(4-nitrophenyl)thiocarbonate rapamycin

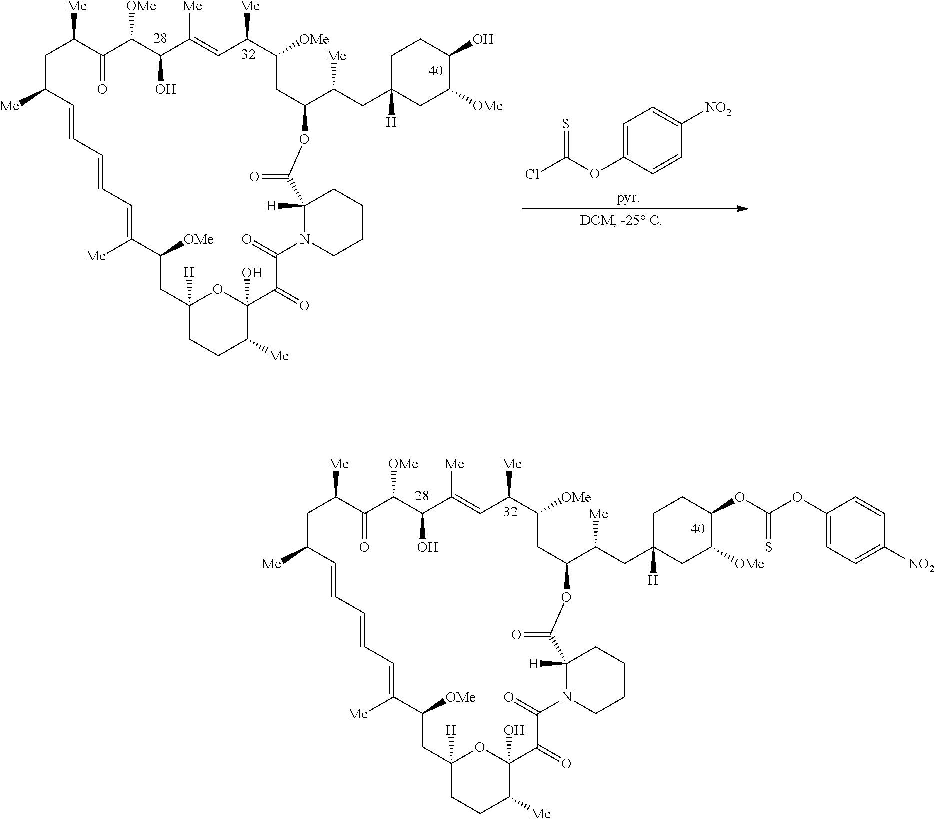 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=O)[C@H](C)C[C@H](C)/C=C/C=C/C=C/1C ; O=[N+]([O-])c1ccc(OC(=S)Cl)cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

To a solution of 32(R)-methoxy rapamycin (6.69 g, 7.19 mmol, 1.0 equiv) in DCM (67 mL) was added pyridine (6.6 mL, 81 mmol, 11 equiv) and dried 4 Å molecular sieves (6.7 g). The suspension was stirred at room temperature for 1 h, at which point the mixture was cooled to −25° C. and a solution of O-(4-nitrophenyl)chlorothioformate (1.88 g, 8.63 mmol, 1.20 equiv) in DCM (13 mL) was added. The reaction was warmed to room temperature and after 21 h was filtered through Celite. The filtrate was partitioned between DCM and H₂O and the aqueous layer was extracted with DCM. The combined organic layers were dried and concentrated under reduced pressure. Purification by silica gel chromatography (EtOAc/hexanes) afforded the desired product as a white solid (5.1 g, 64% yield). LCMS (ESI) m/z: [M+Na] calcd for C₅₉H₈₆N₂O₁₆S: 1133.56; found 1134.1.

### Monomer 9. 32-deoxy 40(R)—O-(4-nitrophenyl)carbonate rapamycin

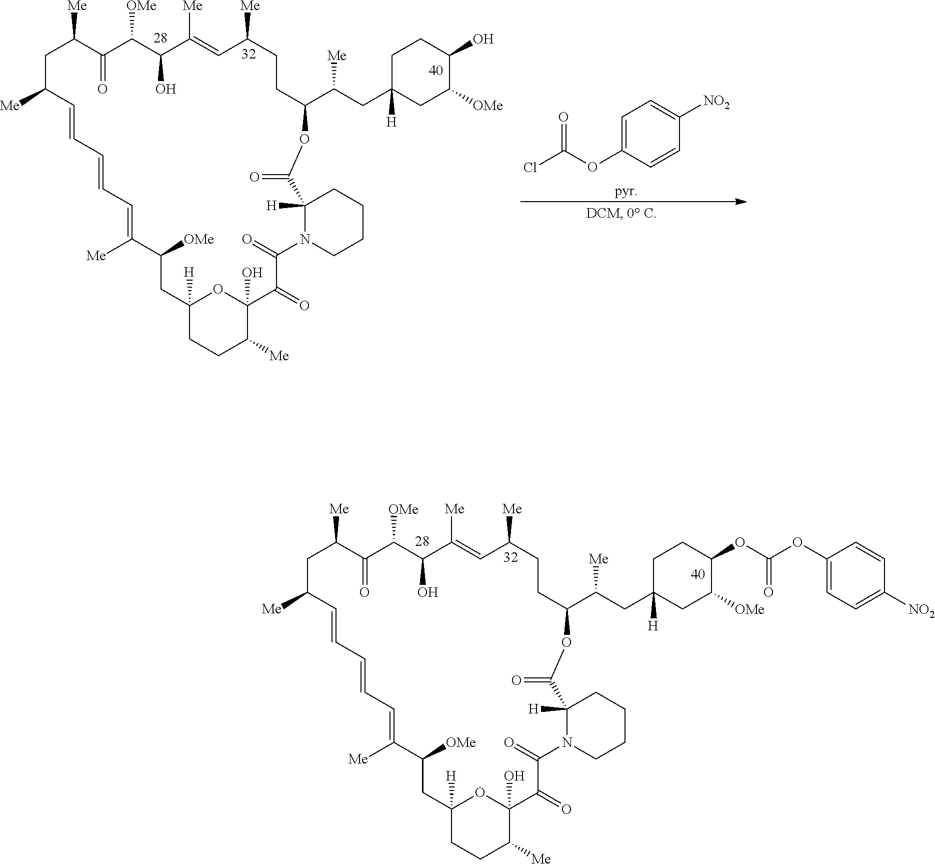 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C ; O=C(Cl)Oc1ccc([N+](=O)[O-])cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)CC[C@H](C)/C=C(\C)[C@@H](O)[C@@H](OC)C(=O)[C@H](C)C[C@H](C)/C=C/C=C/C=C/1C]

To a solution of 32-deoxy rapamycin (0.623 g, 0.692 mmol, 1.0 equiv) in DCM (24.7 mL) was added 4 Å molecular sieves (600 mg). The suspension was stirred for 1 h and then pyridine (557 μL, 6.92 mmol, 10 equiv) was added. The reaction mixture was cooled to 0° C. and then O-(4-nitrophenyl)chloroformate (175 mg, 1.03 mmol, 1.7 equiv) was added. The reaction warmed to room temperature and stirred for 2 h, at which point the reaction mixture was concentrated under reduced pressure. Purification by silica gel chromatography (0→10% MeOH/DCM) afforded the desired product as a white solid (0.61 g, 82% yield). LCMS (ESI) m/z: [M+Na] calcd for C₅₈H₈₄N₂O₁₆: 1087.57; found 1087.6.

### Monomer 10. 32-deoxy 40(R)—O-(4-nitrophenyl)thiocarbonate rapamycin

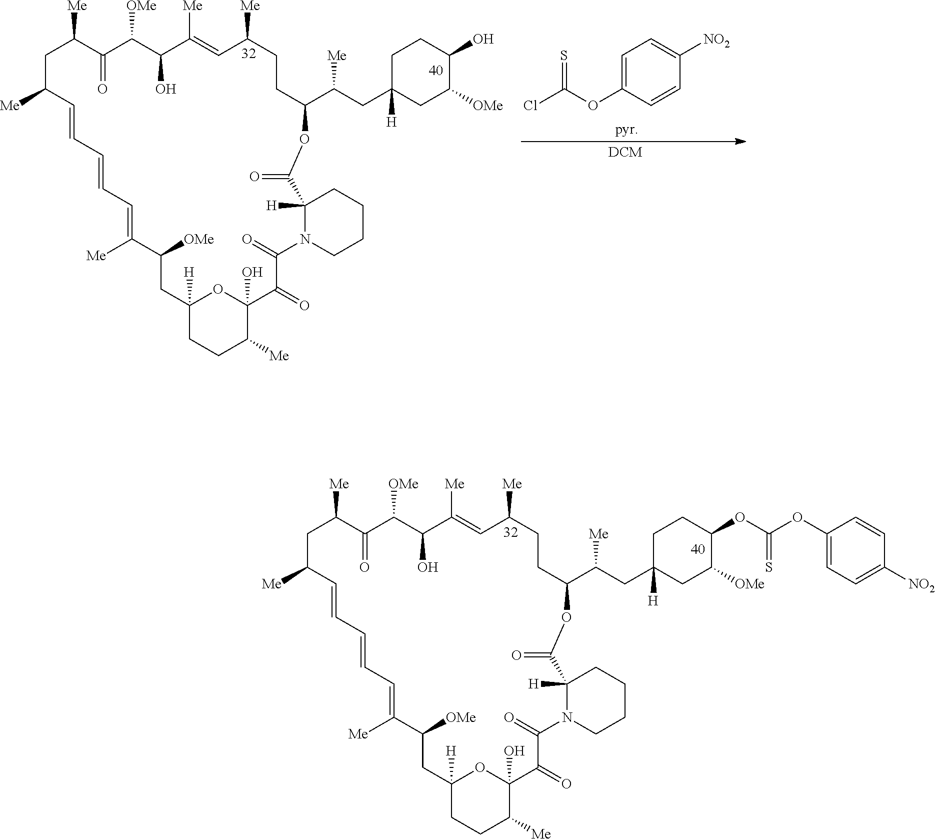 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C ; O=[N+]([O-])c1ccc(OC(=S)Cl)cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)CC[C@H](C)/C=C(\C)[C@@H](O)[C@@H](OC)C(=O)[C@H](C)C[C@H](C)/C=C/C=C/C=C/1C]

To a 0.2 M solution of 32-deoxy rapamycin (1.0 equiv) in DCM at −78° C. is added pyridine (11.2 equiv), followed by a 0.7 M solution of O-(4-nitrophenyl)chlorothiocarbonate (1.3 equiv) in DCM. The reaction mixture is warmed to −20° C. and stirred until consumption of the starting material, as determined by LCMS analysis. Hexane is then added and the resulting suspension is purified by silica gel chromatography to provide the product.

### Monomer 11. 28(R)-methoxy 32(R)-hydroxy 40(R)-(4-nitrophenyl)carbonate rapamycin

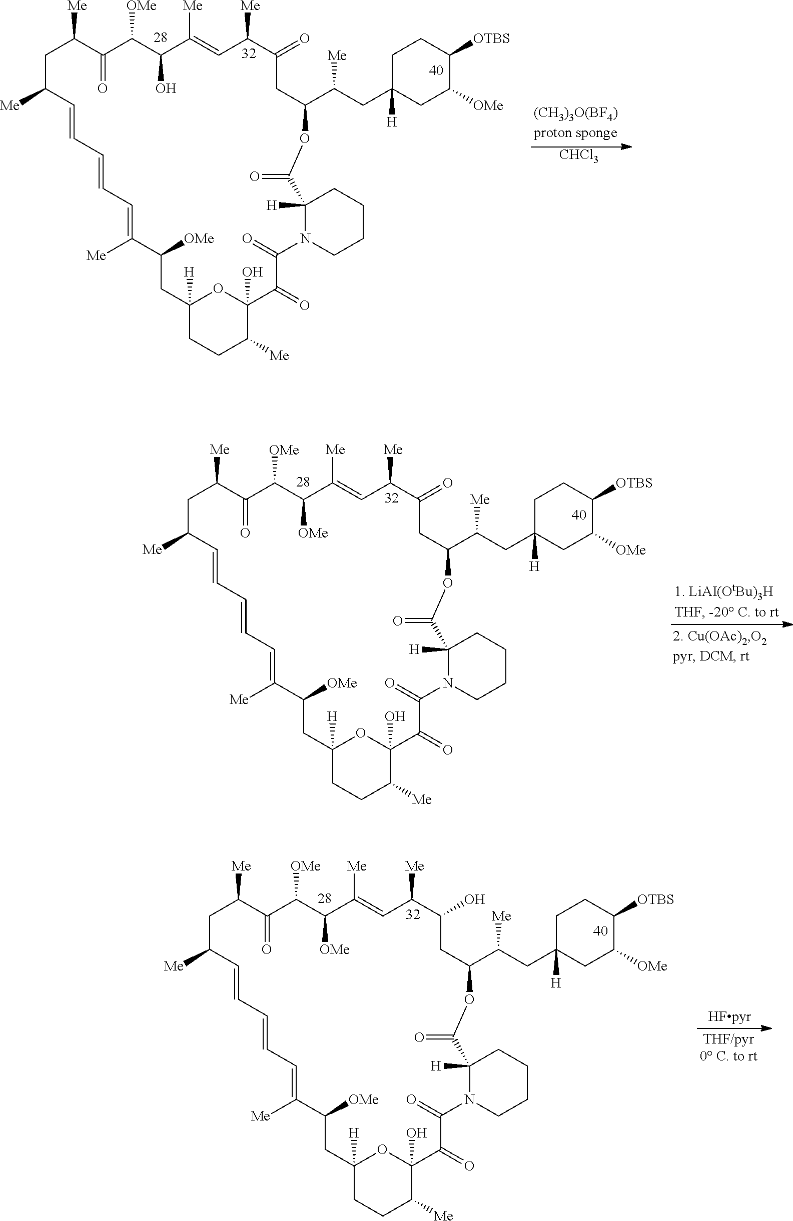

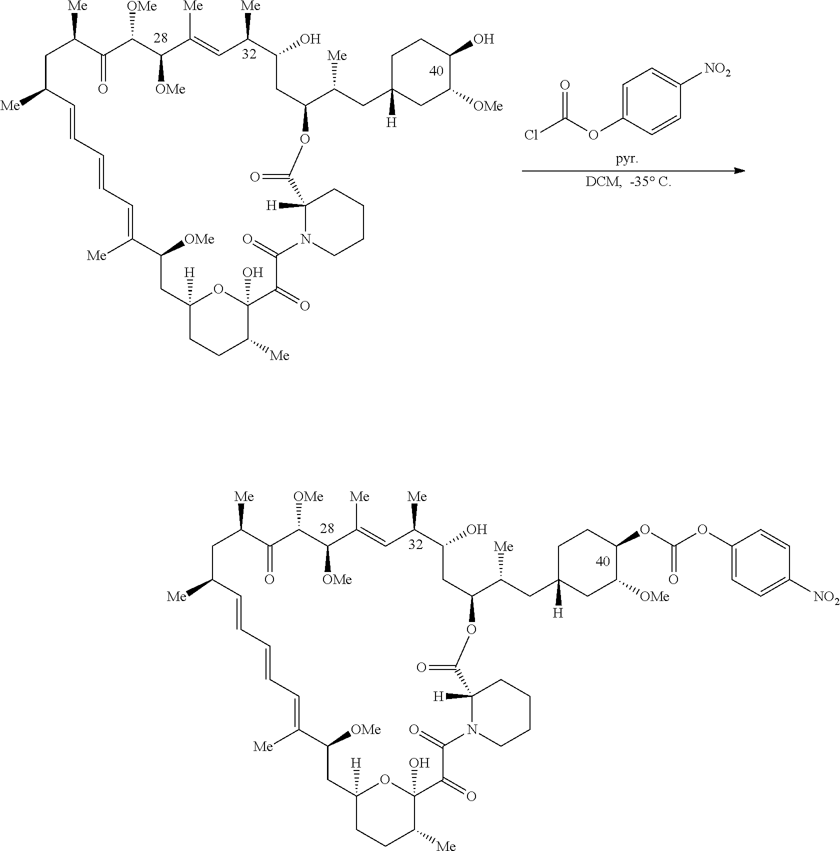 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](OC)[C@@H](OC)C(=O)[C@H](C)C[C@H](C)/C=C/C=C/C=C/1C ; O=C(Cl)Oc1ccc([N+](=O)[O-])cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](OC)[C@@H](OC)C(=O)[C@H](C)C[C@H](C)/C=C/C=C/C=C/1C]

### Step 1: Synthesis of 28(R)-methoxy 40(R)—O-tert-butyldimethylsilyl rapamycin

To a solution o 40(R)—O-tert-butyldimethylsilyl rapamycin (4.00 g, 4.89 mmol, 1.0 equiv) in chloroform (67 mL) was added proton sponge (11.2 mL, 52.3 mmol, 13 equiv) and dried 4 Å molecular sieves (5.8 g). The suspension was stirred at room temperature for 1 h, at which point trimethyloxonium tetrafluoroborate (7.21 g, 48.8 mmol, 12.5 equiv) was added. After 4 h the suspension was filtered through Celite. The filtrate was washed sequentially with aqueous 2 N HCl. H₂O, sat. aqueous NaHCO₃, then dried and concentrated under reduced pressure. Purification by silica gel chromatography (EtOAc/hexane) afforded the desired product as a white solid (2.1 g, 52% yield). LCMS (ESI) m/z: [M+Na] calcd for C₅₈H₉₅NO₁₃Si: 1064.65; found 1065.26.

### Step 2: Synthesis of 28(R)-methoxy 32(R)-hydroxy 40(R)—O-tert-butyldimethylsilyl rapamycin

To a solution of 28(R)-methoxy 40(R)—O-tert-butyldimethylsilyl rapamycin (2.13 g, 2.04 mmol, 1.0 equiv) in THF (31 mL) at −20° C. was added a solution of lithium tri-tert-butoxyaluminum hydride in THF (1 M, 4.09 mL, 4.09 mmol, 2.0 equiv), dropwise. The reaction mixture was warmed to room temperature and after 3 h was added to a solution of H₂O (4 mL). EtOAc (31 mL), and 2M aqueous citric acid (4 mL) at 0° C. After 5 min the mixture was partitioned, and the aqueous layer was extracted with EtOAc. The combined organic layers were poured into a sat. aqueous NaHCO₃ solution (60 mL) at 0° C. The layers were partitioned, and the organic layer was dried and concentrated under reduced pressure to provide a crude white solid (2.32 g). The crude solid was dissolved in DCM (12 mL) and then pyridine (241 μL, 2.98 mmol, 1.5 equiv), dried 4 Å molecular sieves (2.1 g), and cupric acetate (0.27 g, 1.49 mmol, 0.7 equiv) were added. The suspension was stirred at room temperature for 1 h. The suspension was sparged with O₂ and then kept under an O₂ atmosphere for 30 min. After 2 h the mixture was filtered through Celite and the filtrate was concentrated under reduced pressure. Purification by silica gel chromatography (EtOAc/hexane) afforded the desired product as a white solid (307 mg, 14% yield). LCMS (ESI) m/z: [M+Na] calcd for C₅₈H₉₇NO₁₃Si: 1066.66; found 1067.0.

### Step 3: Synthesis of 28(R)-methoxy 32(R)-hydroxy rapamycin

To a solution of 28(R)-methoxy 32(R)-hydroxy 40(R)—O-tert-butyldimethylsilyl rapamycin (0.307 g, 0.294 mmol, 1.0 equiv) in THF (4 mL) in a polypropylene vial at 0° C. was added pyridine (1.42 mL, 17.6 mmol, 60.0 equiv) followed by 70% HF-pyridine (0.34 mL, 11.7 mmol, 40 equiv). The solution was warmed to room temperature and stirred for 21 h, at which point the solution was poured into sat. aqueous NaHCO₃ at 0° C. The aqueous layer was extracted with EtOAc (2×) and the combined organic layers were washed with sat. aqueous NaHCO₃ and brine, then dried and concentrated under reduced pressure. Purification by silica gel chromatography (EtOAc/hexane) afforded the desired product as a white solid (146 mg, 53% yield). LCMS (ESI) m/z: [M+Na] calcd for C₅₂H₈₃NO₁₃: 952.58; found 952.8.

### Step 4: Synthesis of 28(R)-methoxy 32(R)-hydroxy 40(R)-(4-nitrophenyl)carbonate rapamycin

To a solution of 28(R)-methoxy 32(R)-hydroxy rapamycin (0.66 g, 0.71 mmol, 1.0 equiv) in DCM (3 mL) was added pyridine (0.64 mL, 7.9 mmol, 11 equiv) and dried 4 Å molecular sieves (0.66 g). The suspension was stirred at room temperature for 1 h, at which point the mixture was cooled to −35° C. and O-(4-nitrophenyl)chloroformate (0.17 g, 0.85 mmol, 1.2 equiv) was added. After 3 h, DCM (5 mL) was added and the suspension was filtered through Celite. The filtrate was washed with H₂O, dried, and concentrated under reduced pressure. Purification by silica gel chromatography (EtOAc/hexanes) afforded the desired product as a white solid (0.44 g, 57% yield). LCMS (ESI) m/z: [M+Na] calcd for C₅₉H₈₆N₂O₁₇: 1117.58; found 1118.0.

### Monomer 12. 28(R)-methoxy 32(R)-methoxy 40(R)-(4-nitrophenyl)carbonate rapamycin

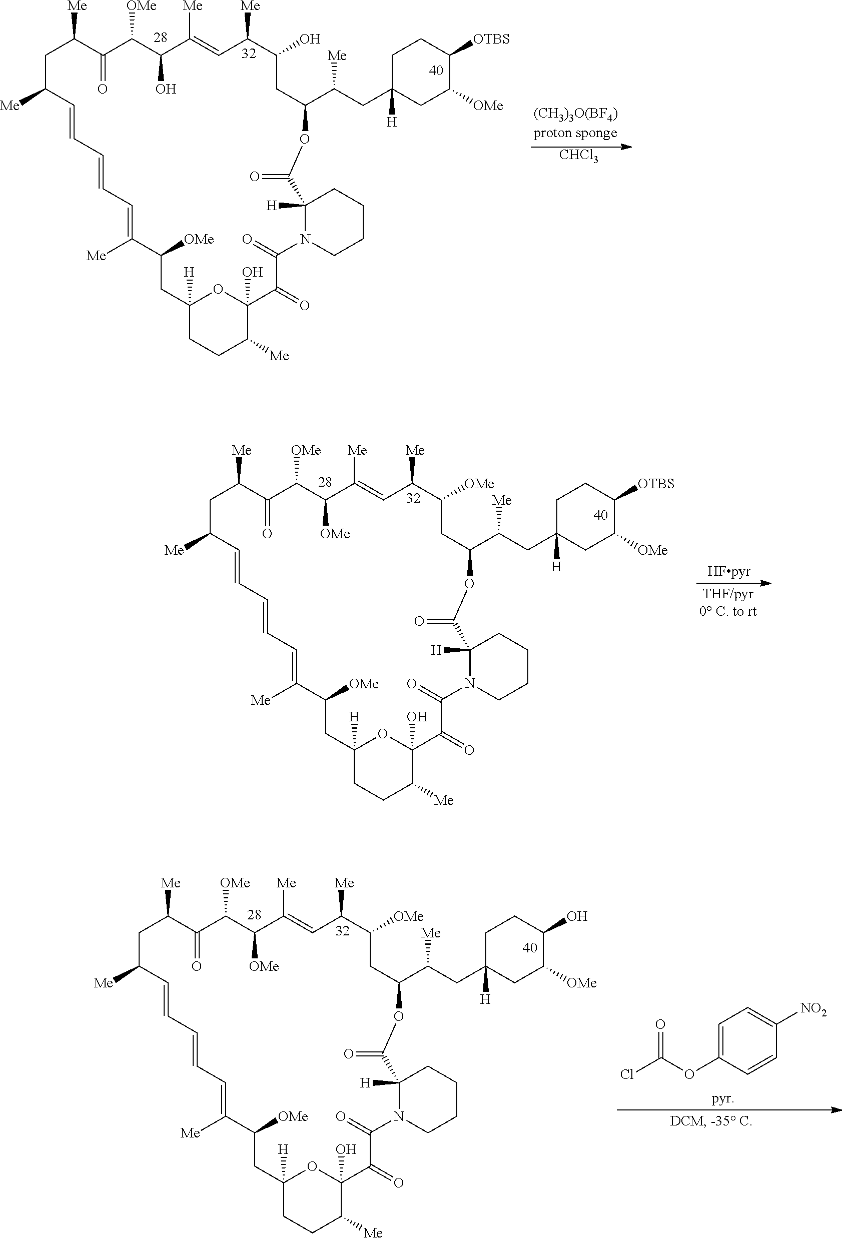

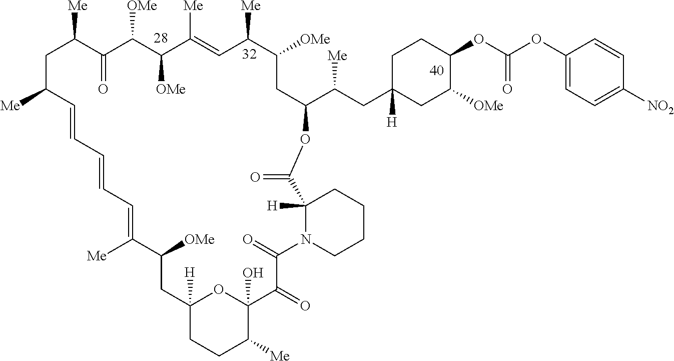 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](OC)[C@H](C)/C=C(\C)[C@@H](OC)[C@@H](OC)C(=O)[C@H](C)C[C@H](C)/C=C/C=C/C=C/1C]

### Step 1: Synthesis of 28(R)-methoxy 32(R)-methoxy 40(R)—O-tert-butyldimethylsilyl rapamycin

To a solution of 28(R)-methoxy 32(R)-hydroxy 40(R)—O-tert-butyldimethylsilyl rapamycin (1.15 g, 1.10 mmol, 1.0 equiv) in chloroform (19 mL) was added proton sponge (3.22 mL, 15.0 mmol, 14 equiv) and dried 4 Å molecular sieves (2.3 g). The suspension was stirred at room temperature for 1 h, at which point trimethyloxonium tetrafluoroborate (2.07 g, 14.0 mmol, 12.7 equiv) was added. After 4 h the suspension was filtered through Celite and the filtrate was washed with 1N HCl, H₂O, and sat. aqueous NaHCO₃, dried and concentrated under reduced pressure. Purification by silica gel chromatography (EtOAc/hexane) afforded the desired product as a white solid. LCMS (ESI) m/z: [M+Na] calcd for C₅₉H₉₉NO₃Si: 1080.68; found 1081.2.

### Step 2: Synthesis of 28(R)-methoxy 32(R)-methoxy rapamycin

To a solution of 28(R)-methoxy 32(R)-methoxy 40(R)—O-tert-butyldimethylsilyl rapamycin in THF (4 mL) in a polypropylene vial at 0° C. was added pyridine (1.13 mL, 14.2 mmol, 12.9 equiv) followed by 70% HF-pyridine (0.27 mL, 9.42 mmol, 8.6 equiv). The solution was warmed to room temperature and stirred for 41 h, at which point the solution was poured into sat. aqueous NaHCO₃ at 0° C. The aqueous layer was extracted with EtOAc (2×) and the combined organic layers were washed with sat. aqueous NaHCO₃ and brine, then dried and concentrated under reduced pressure. Purification by silica gel chromatography (EtOAc/hexane) afforded the desired product as a white solid (516 mg, 49% yield 2-steps). LCMS (ESI) m/z: [M+Na] calcd for C₅₃H₈₅NO₁₃: 966.59; found 967.0.

### Step 3: Synthesis of 28(R)-methoxy 32(R)-methoxy 40(R)-(4-nitrophenyl)carbonate rapamycin

To a solution of 28(R)-methoxy 32(R)-methoxy rapamycin (0.30 g, 0.32 mmol, 1.0 equiv) in DCM (1.4 mL) was added pyridine (0.29 mL, 3.5 mmol, 11 equiv) and dried 4 Å molecular sieves (0.30 g). The suspension was stirred at room temperature for 1 h, at which point it was cooled to −35° C. and O-(4-nitrophenyl)chloroformate (0.08 g, 0.38 mmol, 1.2 equiv) was added. After 3 h. DCM (2 mL) was added and the suspension was filtered through Celite. The filtrate was washed with H₂O, dried and concentrated under reduced pressure. Purification by silica gel chromatography (EtOAc/hexanes) afforded the desired product as an off-white solid (0.20 g, 57% yield). LCMS (ESI) m/z: [M+Na] calcd for C₆₀H₈₈N₂O₁₇: 1131.60; found 1132.1.

### Monomer 13. 32(R)-(4-nitrophenyl)carbonate rapamycin

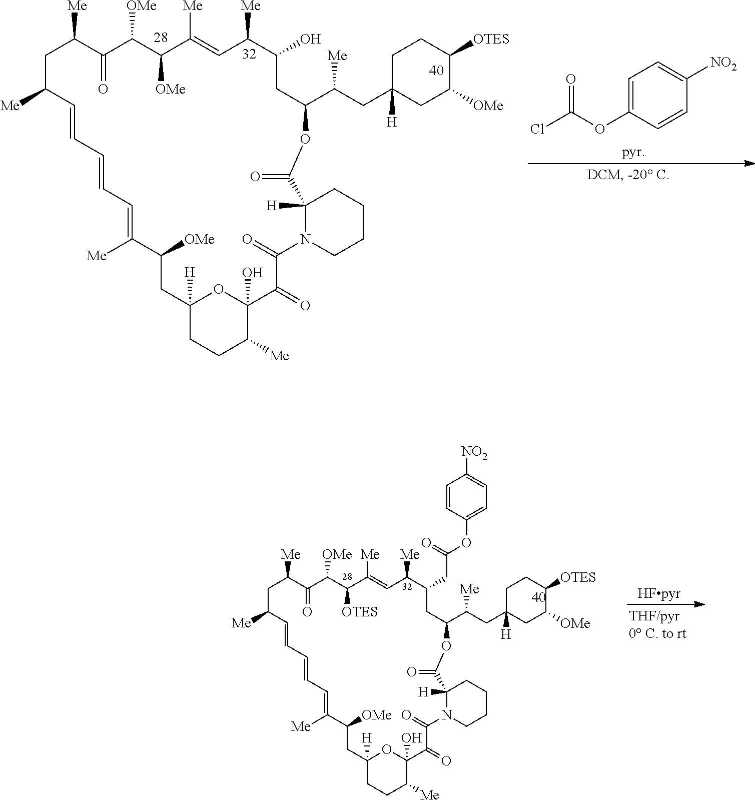

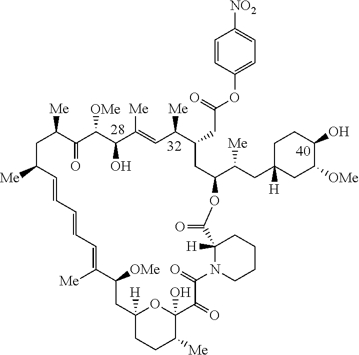 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](CC(=O)Oc2ccc([N+](=O)[O-])cc2)[C@H](C)/C=C(\C)[C@@H](O)[C@@H](OC)C(=O)[C@H](C)C[C@H](C)/C=C/C=C/C=C/1C]

### Step 1: Synthesis of 28,40-O-bis(triethylsilyl) 32(R)-(4-nitrophenyl)carbonate rapamycin

To a solution of 28,40-O-bis(triethylsilyl) 32(R)-hydroxy rapamycin (0.602 g, 0.526 mmol, 1.0 equiv) in DCM (16 mL) at −20° C. was added pyridine (0.82 mL, 10 mmol, 19 equiv) followed by O-(4-nitrophenyl)chloroformate (0.36 g, 1.8 mmol, 3.4 equiv). The reaction mixture was warmed to room temperature and stirred for 1 h, at which point the solution was diluted with DCM (50 mL) and poured into H₂O (30 mL). The aqueous layer was extracted with DCM (50 mL) and the combined organic layers were washed with brine (20 mL), dried and concentrated under reduced pressure to afford a faint yellow foam that was used directly in the next step.

### Step 2: Synthesis of 32(R)-(4-nitrophenyl)carbonate rapamycin

To a solution of 28,40-O-bis(triethylsilyl) 32(R)-(4-nitrophenyl)carbonate rapamycin in THF (10 mL) in a polypropylene vial at 0° C. was added pyridine (1.70 mL, 21.0 mmol, 40.0 equiv) followed by 70% HF-pyridine (0.46 mL, 15.8 mmol, 30.0 equiv). The solution was warmed to room temperature and stirred overnight, at which point the solution was poured into sat. aqueous NaHCO₃ at 0° C. The aqueous layer was extracted with EtOAc (3×) and the combined organic layers were washed with sat. aqueous NaHCO₃ and brine, then dried and concentrated under reduced pressure. Purification by reverse phase chromatography (20→100% MeCN/H₂O) afforded the desired product as an off-white powder (420 mg, 74% yield 2-steps). LCMS (ESI) m/z: [M+Na] calcd for C₅₈H₈₄N₂O₁₇: 1103.57; found 1104.0.

### Monomer 14. 32(S)-azido 40-(4-nitrophenyl)carbonate rapamycin

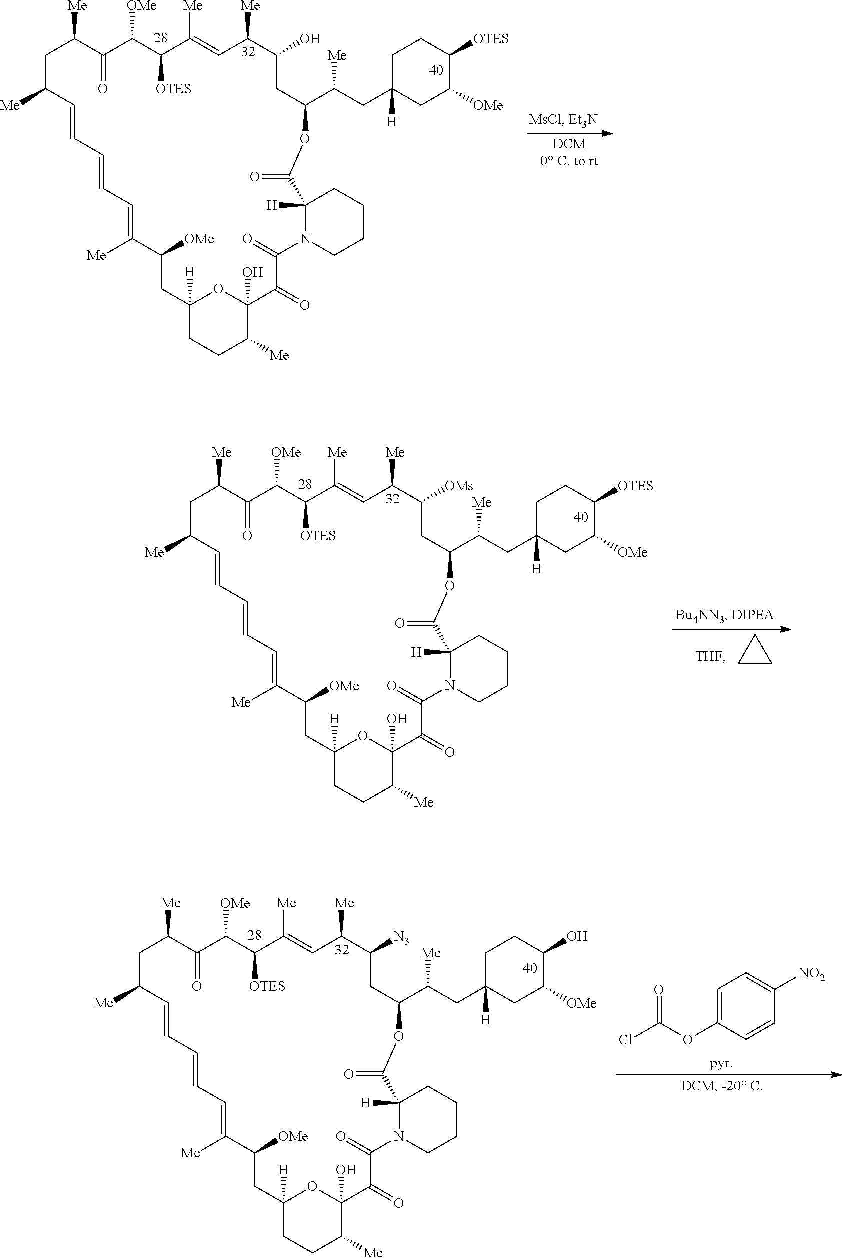

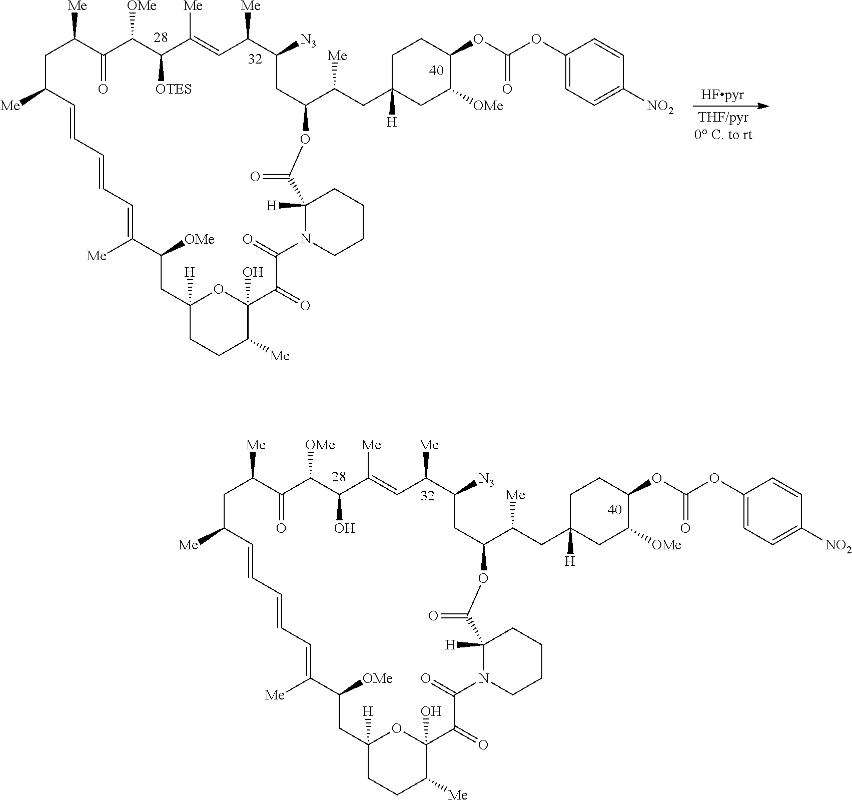

### Step 1: Synthesis of 28,40-O-bis(triethylsilyl) 32(R)—O-methanesulfonyl rapamycin

To a solution of 28,40-O-bis(triethylsilyl) 32(R)-hydroxy rapamycin (2.50 g, 2.18 mmol, 1.0 equiv) in DCM (25 mL) at 0° C. was added Et₃N (0.912 mL, 6.54 mmol, 3.0 equiv) followed by methanesulfonyl chloride (0.338 mL, 4.36 mmol, 2.0 equiv). The solution was stirred at 0° C. for 3 h, at which point the EtOAc was added and the solution was washed with sat. aqueous NaHCO₃. The combined organic layers were washed with brine, dried and concentrated under reduced pressure to give a yellow oil which was used directly in the next step.

### Step 2: Synthesis of 28-O-triethylsilyl 32(S)-azido rapamycin

To a solution of 28,40-O-bis(triethylsilyl) 32(R)—O-methanesulfonyl rapamycin in THF (40 mL) was added DIPEA (0.761 mL, 4.37 mmol, 2.0 equiv) and tetrabutylammonium azide (3.72 g, 13.1 mmol, 6.0 equiv). The reaction solution heated to reflux for 5.5 h and then cooled to room temperature. The solution was diluted with EtOAc and washed with sat. aqueous NaHCO₃. The combined organic layers were washed with brine, dried and concentrated under reduced pressure. Purification by reverse phase chromatography (30→100% MeCN/H₂O) afforded the desired product as a clear glass (746 mg, 33% yield 2-steps). LCMS (ESI) m/z: [M+Na] calcd for C₅₇H₉₄N₄O₁₂Si: 1077.65; found 1077.8.

### Step 3: Synthesis of 28-O-triethylsilyl 32(S)-azido 40(R)-(4-nitrophenyl)carbonate rapamycin

To a solution of 28-O-triethylsilyl 32(S)-azido rapamycin (0.505 g, 0.478 mmol, 1.0 equiv) in DCM (15 mL) was added pyridine (0.75 mL, 9.3 mmol, 19 equiv) and 4 Å molecular sieves. The suspension was cooled to −20° C. and O-(4-nitrophenyl)chloroformate (0.32 g, 1.6 mmol, 3.4 equiv) was added. The suspension was stirred at −20° C. for 2 h, at which point the it was diluted with DCM (50 mL), filtered and poured into H₂O (20 mL). The aqueous layer was extracted with DCM (50 mL) and the combined organic layers were washed with brine (20 mL), dried and concentrated under reduced pressure to give a pale-yellow foam which was used directly in the next step.

### Step 4: Synthesis of 32(S)-azido 40-O-(4-nitrophenyl)carbonate rapamycin

To a solution of 28-O-triethylsilyl 32(S)-azido 40(R)-(4-nitrophenyl)carbonate rapamycin in THF (10 mL) in a polypropylene vial at 0° C. was added pyridine (1.55 mL, 19.1 mmol, 40.0 equiv) followed by 70% HF-pyridine (0.42 mL, 14.4 mmol; 30.0 equiv). The solution was warmed to room temperature and stirred overnight, at which point the solution was poured into sat. aqueous NaHCO₃ at 0° C. The aqueous layer was extracted with EtOAc (3×) and the combined organic layers were washed with sat. aqueous NaHCO₃ and brine, then dried and concentrated under reduced pressure. Purification by reverse phase chromatography (30→100% MeCN/H₂O) afforded the desired product as an off-white powder (410 mg, 77% yield 2-steps). LCMS (ESI) m/z: [M+Na] calcd for C₅₈H₈₃N₅O₁₆: 1128.57; found 1129.0.

### Monomer 15. 28-methoxy-40-O-(4-nitrophenoxy)carbonyl rapamycin

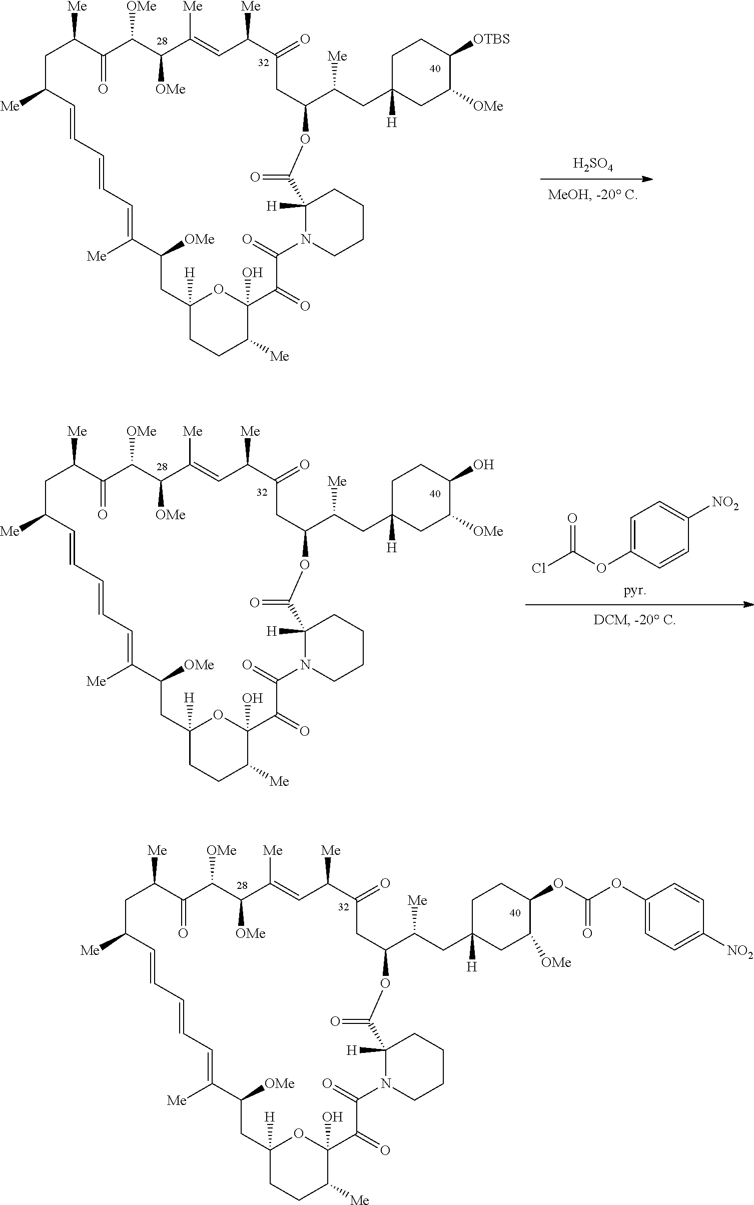

### Step 1: Synthesis of 28-methoxy rapamycin

To a solution of 28-methoxy-40-O-(tert-butyldimethyl)silyl rapamycin (0.500 g, 0.480 mmol, 1.0 equiv) in MeOH (1.6 mL) at −20° C. was added H₂SO₄ (1.28 μL, 0.024 mmol, 0.05 equiv). The reaction mixture was stirred at −20° C. for 48 h. The reaction mixture was then poured into sat. aqueous NaHCO₃ (4 mL)/H₂O (4 mL). The aqueous layer was extracted with MTBE (2×6 mL), and the combined organic phases were dried, filtered, and concentrated under reduced pressure. Purification by reverse phase chromatography (30→100% MeCN/H₂O) afforded the desired product as a yellow powder (270 mg, 61% yield). LCMS (ESI) m/z: [M+Na] calcd for C₅₂H₈₁NO₁₃: 950.5; found 950.7.

### Step 2: Synthesis of 28-methoxy-40-O-(4-nitrophenoxy)carbonyl rapamycin

To a solution of 28-methoxy rapamycin (0.210 g, 0.226 mmol, 1.0 equiv) in DCM (7.1 mL) at −20° C. was added pyridine (0.35 mL, 4.4 mmol, 19 equiv) and then p-nitrophenyl chloroformate (0.15 g, 0.76 mmol, 3.4 equiv). The reaction mixture was stirred at −20° C. for 30 min and then warmed to room temperature. After stirring overnight, p-nitrophenyl chloroformate (0.15 g, 0.76 mmol, 3.4 equiv) was added and the reaction was stirred for an additional 2 h. The reaction mixture was diluted with DCM (20 mL) and poured into H₂O (10 mL). The aqueous layer was extracted with DCM (20 mL), and the combined organic layers were washed with brine (9 mL), dried, filtered and concentrated under reduced pressure. Purification by reverse phase chromatography (50→100% MeCN/H₂O) afforded the desired product as a pale yellow powder (200 mg, 81% yield). LCMS (ESI) m/z: [M+Na] calcd for C₅₉H₈₄N₂O₁₇: 1115.6; found 1115.8.

### Monomer 16. 32(R),40-O,O-bis[(4-nitrophenoxy)carbonyl]rapamycin

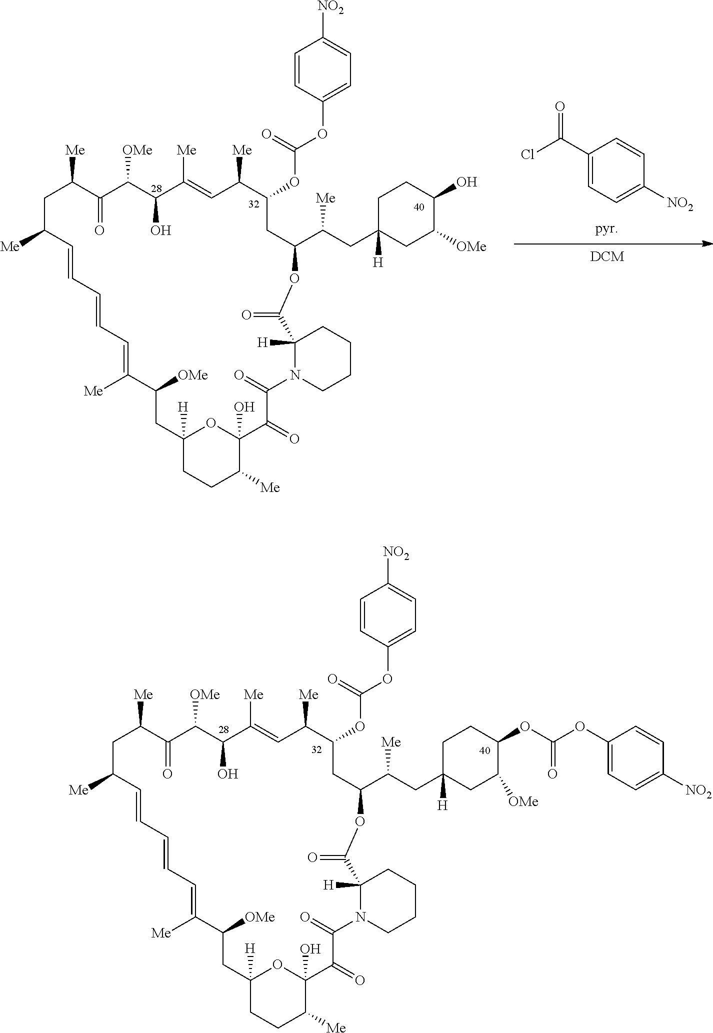

To a solution of 32(R)—O-[(4-nitrophenoxy)carbonyl] rapamycin (675 mg, 0.624 mmol, 1.0 equiv) in DCM (13 mL) was added powdered 4 Å molecular sieves (675 mg). The suspension was stirred for 1 h, at which point pyridine (0.56 mL, 6.90 mmol, 11.1 equiv) was added. The mixture was cooled to −15° C. and then p-nitrophenyl chloroformate (132 mg, 0.655 mmol, 1.05 equiv) was added in one portion. The mixture was warmed to 0° C., stirred for 4 h, and then warmed to room temperature. The reaction mixture was filtered and washed with DCM (25 mL). The filtrate was washed with sat. aqueous NaHCO₃ (15 mL), H₂O (15 mL), and brine (10 mL), dried, filtered, and concentrated under reduced pressure. Purification by silica gel chromatography (25→45% EtOAc/hexanes) afforded the desired product as a faint yellow solid (566 mg, 73% yield). LC-MS (ESI) m/z: [M+Na] calcd for C₆₅H₈₇N₃O₂₁: 1268.57; found 1269.3.

### Monomer 17. 28(R),32(R),40(R)—O,O,O-tris[(4-nitrophenoxy)carbonyl]rapamycin

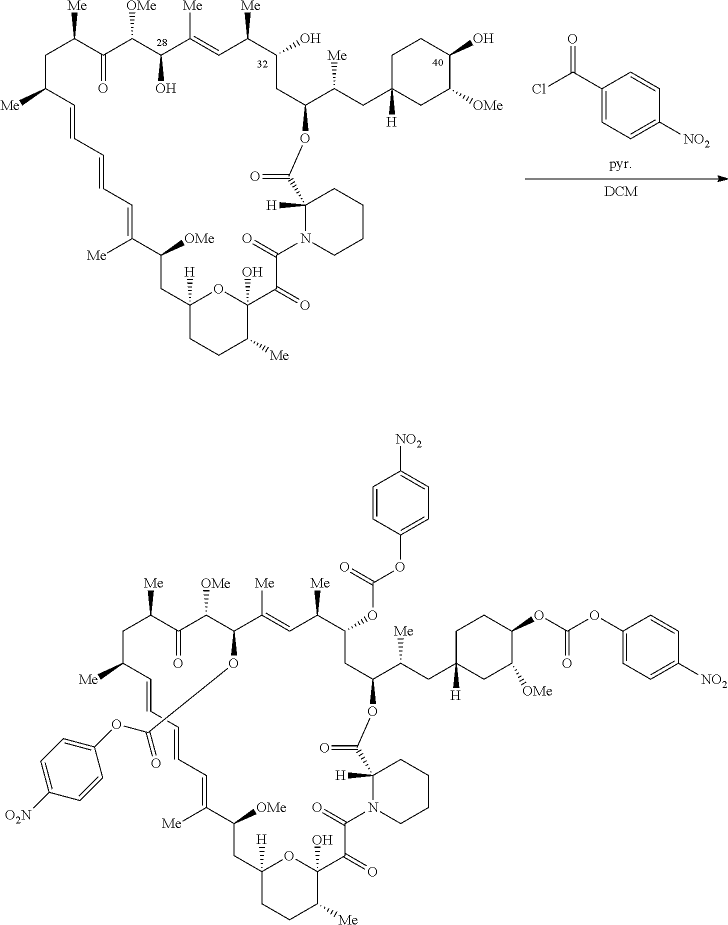

To a solution of 32(R)-hydroxy rapamycin (1.00 g, 1.09 mmol, 1.0 equiv) in DCM (22 mL) was added powdered 4 Å molecular sieves (1.0 g). The suspension was stirred for 45 min, at which point pyridine (0.97 mL, 12.0 mmol, 11.0 equiv) was added. The mixture was cooled to −15° C. and then p-nitrophenyl chloroformate (550 mg, 2.73 mmol, 2.5 equiv) was added in one portion. The mixture was warmed to room temperature over 4 h and stirred overnight. The mixture was cooled to 0° C. and additional p-nitrophenyl chloroformate (220 mg, 1.09 mmol, 1.0 equiv) was added in one portion. The reaction mixture was stirred for 1 h, warmed to room temperature, and then stirred for 2 h. The mixture was once again cooled to 0° C. and additional p-nitrophenyl chloroformate (660 mg, 3.27 mmol, 3.0 equiv) was added. The reaction mixture was stirred for 15 min and then at room temperature for 1 h. The reaction mixture was filtered and washed with DCM (25 mL). The filtrate was washed with sat. aqueous NaHCO₃ (20 mL), H₂O (20 mL), and brine (15 mL), dried, filtered, and concentrated under reduced pressure. Purification by silica gel chromatography (5→15% EtOAc/DCM) afforded the desired product as a faint yellow solid (550 mg, 36% yield). LC-MS (ESI) m/z: [M+Na] calcd for C₇₂H₉₀N₄O₂₅: 1433.58; found 1434.3.

### General Procedures and Specific Examples

### General Procedure 1: Coupling of a Carboxylic Acid and an Amine Followed by N-Boc Deprotection

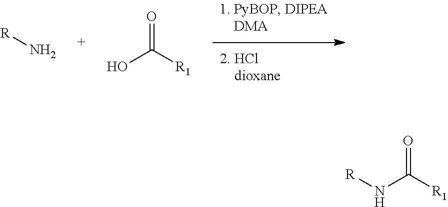

### Step 1

To a 0.1 M solution of carboxylic acid (1.0 equiv) in DMA was added an amine (1.2 equiv), DIPEA (4.0 equiv) and PyBOP (1.3 equiv). The reaction was allowed to stir until consumption of the carboxylic acid, as indicated by LCMS. The reaction mixture was then purified by silica gel chromatography to afford the product.

### Step 2

To a 0.07 M solution of N-Boc protected amine (1.0 equiv) in dioxane was added HCl (4 M in dioxane) (50 equiv). The reaction was allowed to stir until consumption of N-Boc protected amine, as indicated by LCMS. Then the reaction was concentrated to an oil, which was then dissolved in H₂O and lyophilized to afford the product.

### Intermediate A1-7. 1-amino-27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-3,6,9,12,15,18,21,24-octaoxaheptacosan-27-one

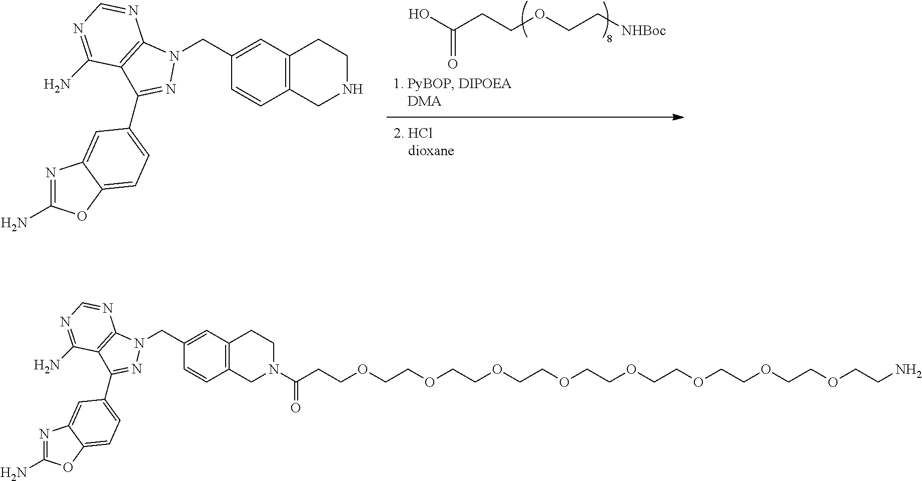

### Step 1: Synthesis of tert-butyl N-[27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-27-oxo-3,6,9,12,15,18,21,24-octaoxaheptacosan-1-yl]carbamatecarbamate

To a solution of 1-{[(tert-butoxy)carbonyl]amino}-3,6,9,12,15,18,21,24-octaoxaheptacosan-27-oic acid (102 mg, 189 μmol, 1.0 equiv) and 6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-ium (120 mg, 227 μmol, 1.2 equiv) in DMA (1.88 mL) was added DIPEA (131 μL, 756 μmol, 4.0 equiv) followed by PyBOP (127 mg, 245 μmol, 1.3 equiv). The reaction was stirred at room temperature. After 2 h, the reaction mixture was concentrated under reduced pressure and the crude residue was purified by silica gel chromatography (0→20% MeOH/DCM) to give the product (161.5 mg, 91% yield) as a pale yellow oil. LCMS (ESI) m/z: [M+H] calcd for C₄₆H₆₅N₉O₁₂: 936.49; found 936.3.

### Step 2: Synthesis of 1-amino-27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-3,6,9,12,15,18,21,24-octaoxaheptacosan-27-one

To a solution of tert-butyl N-[27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-27-oxo-3,6,9,12,15,18,21,24-octaoxaheptacosan-1-yl]carbamate (0.9 g, 0.9614 mmol, 1.0 equiv) in dioxane (3.20 mL) was added HCl (4 M in dioxane, 2.40 mL, 9.61 mmol, 10.0 equiv). The reaction stirred for 2 h and then was concentrated under reduced pressure to an oil. The oil was azeotroped with DCM (3×15 mL) to provide the product (881 mg, 105% yield, HCl) as a tan solid, which was used directly in the next step. LCMS (ESI) m/z: [M+H] calcd for C₄₁H₅₇N₉O₁₀: 836.43; found 836.3.

Following General Procedure 1, but using the appropriate amine-containing active site inhibitor in Table 2 and PEG carboxylic acid, the Intermediates A 1 in Table 5 were prepared:

TABLE 5
Additional amines prepared

| | | Calcu- | Obser- |
|---|---|---|---|
| | Molecular | lated | ved |
| Structure | Formula | MW | MW |
| 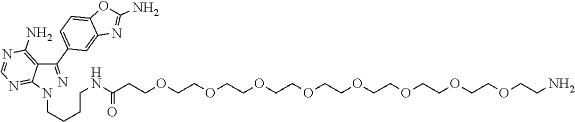 [SMILES: NCCOCCOCCOCCOCCOCCOCCOCCOCCC(=O)NCCCCn1nc(-c2ccc3oc(N)nc3c2)c2c(N)ncnc21] Intermediate A1-1 | C₃₅H₅₅N₉O₁₀ | [M + H] = 762.42 | [M + H] = 762.3 |
| 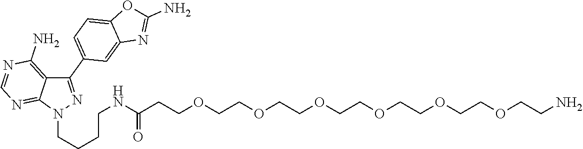 [SMILES: NCCOCCOCCOCCOCCOCCOCCC(=O)NCCCCn1nc(-c2ccc3oc(N)nc3c2)c2c(N)ncnc21] Intermediate A1-2 | C₃₁H₄₇N₉O₈ | [M + H] = 674.36 | [M + H] = 674.3 |
| 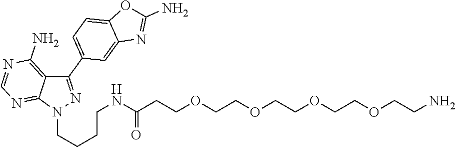 [SMILES: NCCOCCOCCOCCOCCC(=O)NCCCCn1nc(-c2ccc3oc(N)nc3c2)c2c(N)ncnc21] Intermediate A1-3 | C₂₇H₃₉N₉O₆ | [M + H] = 586.31 | [M + H] = 586.6 |
| 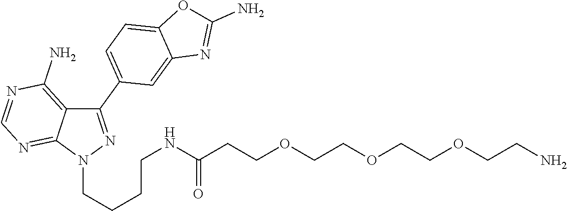 [SMILES: NCCOCCOCCOCCC(=O)NCCCCn1nc(-c2ccc3oc(N)nc3c2)c2c(N)ncnc21] Intermediate A1-4 | C₂₅H₃₅N₉O₅ | [M + H] = 542.29 | [M + H] = 542.3 |
| 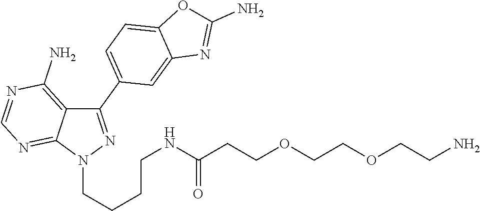 [SMILES: NCCOCCOCCC(=O)NCCCCn1nc(-c2ccc3oc(N)nc3c2)c2c(N)ncnc21] Intermediate A1-5 | C₂₃H₃₁N₉O₄ | [M + H] = 498.26 | [M + H] = 498.2 |
| 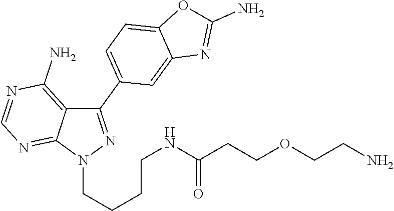 [SMILES: NCCOCCC(=O)NCCCCn1nc(-c2ccc3oc(N)nc3c2)c2c(N)ncnc21] Intermediate A1-6 | C₂₁H₂₇N₉O₃ | [M + H] = 454.23 | [M + H] = 454.1 |
| 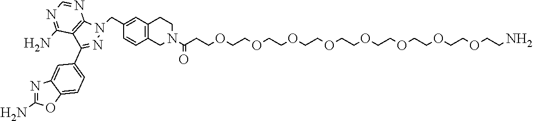 [SMILES: NCCOCCOCCOCCOCCOCCOCCOCCOCCC(=O)N1CCc2cc(Cn3nc(-c4ccc5oc(N)nc5c4)c4c(N)ncnc43)ccc2C1] Intermediate A1-7 | C₄₁H₅₇N₉O₁₀ | [M + H] = 836.43 | [M + H] = 836.3 |
| 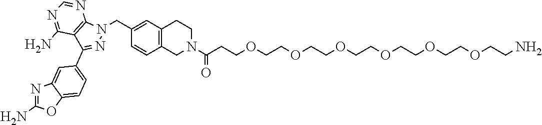 [SMILES: NCCOCCOCCOCCOCCOCCOCCC(=O)N1CCc2cc(Cn3nc(-c4ccc5oc(N)nc5c4)c4c(N)ncnc43)ccc2C1] Intermediate A1-8 | C₃₇H₄₉N₉O₈ | [M + H] = 748.38 | [M + H] = 748.2 |
| 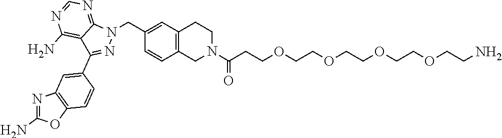 [SMILES: NCCOCCOCCOCCOCCC(=O)N1CCc2cc(Cn3nc(-c4ccc5oc(N)nc5c4)c4c(N)ncnc43)ccc2C1] Intermediate A1-9 | C₃₃H₄₁N₉O₆ | [M + H] = 660.33 | [M + H] = 660.2 |
| 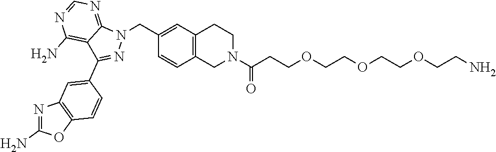 [SMILES: NCCOCCOCCOCCC(=O)N1CCc2cc(Cn3nc(-c4ccc5oc(N)nc5c4)c4c(N)ncnc43)ccc2C1] Intermediate A1-10 | C₃₁H₃₇N₉O₅ | [M + H] = 616.30 | [M + H] = 616.3 |
| 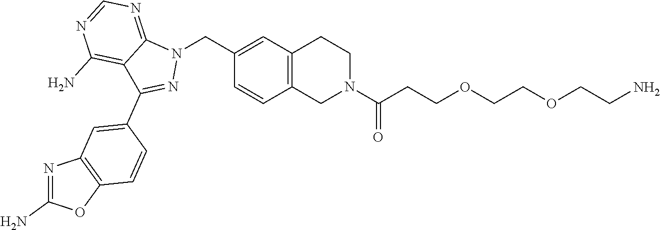 [SMILES: NCCOCCOCCC(=O)N1CCc2cc(Cn3nc(-c4ccc5oc(N)nc5c4)c4c(N)ncnc43)ccc2C1] Intermediate A1-11 | C₂₉H₃₃N₉O₄ | [M + H] = 572.28 | [M + H] = 572.3 |
| 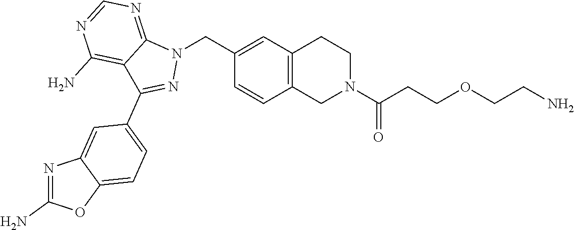 [SMILES: NCCOCCC(=O)N1CCc2cc(Cn3nc(-c4ccc5oc(N)nc5c4)c4c(N)ncnc43)ccc2C1] Intermediate A1-12 | C₂₇H₂₉N₉O₃ | [M + H] = 528.25 | [M + H] = 528.2 |
| 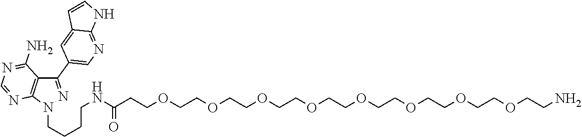 [SMILES: NCCOCCOCCOCCOCCOCCOCCOCCOCCC(=O)NCCCCn1nc(-c2cnc3[nH]ccc3c2)c2c(N)ncnc21] Intermediate A1-13 | C₃₅H₅₅N₉O₉ | [M + H] = 746.42 | [M + H] = 746.4 |
| 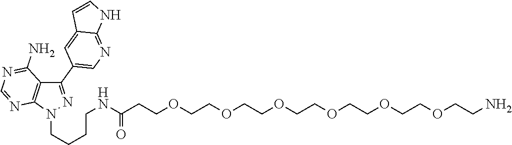 [SMILES: NCCOCCOCCOCCOCCOCCOCCC(=O)NCCCCn1nc(-c2cnc3[nH]ccc3c2)c2c(N)ncnc21] Intermediate A1-14 | C₃₁H₄₇N₉O₇ | [M + H] = 658.37 | [M + H] = 658.3 |
| 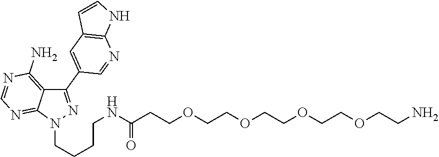 [SMILES: NCCOCCOCCOCCOCCC(=O)NCCCCn1nc(-c2cnc3[nH]ccc3c2)c2c(N)ncnc21] Intermediate A1-15 | C₂₇H₃₉N₉O₅ | [M + H] = 570.32 | [M + H] = 570.2 |
| 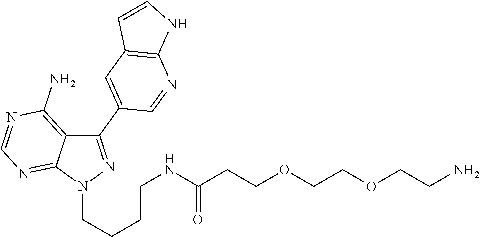 [SMILES: NCCOCCOCCC(=O)NCCCCn1nc(-c2cnc3[nH]ccc3c2)c2c(N)ncnc21] Intermediate A1-16 | C₂₃H₃₁N₉O₃ | [M + H] = 482.26 | [M + H] = 482.3 |
| 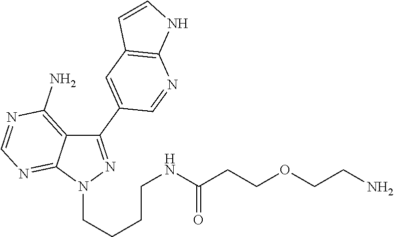 [SMILES: NCCOCCC(=O)NCCCCn1nc(-c2cnc3[nH]ccc3c2)c2c(N)ncnc21] Intermediate A1-17 | C₂₁H₂₇N₉O₂ | [M + H] = 438.24 | [M + H] = 458.4 |
| 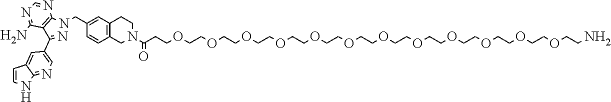 [SMILES: NCCOCCOCCOCCOCCOCCOCCOCCOCCOCCOCCOCCOCCC(=O)N1CCc2cc(Cn3nc(-c4cnc5[nH]ccc5c4)c4c(N)ncnc43)ccc2C1] Intermediate A1-18 | C₄₉H₇₃N₉O₁₃ | [M + H] = 996.54 | [M + H] = 996.4 |
| 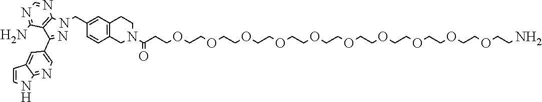 [SMILES: NCCOCCOCCOCCOCCOCCOCCOCCOCCOCCOCCC(=O)N1CCc2cc(Cn3nc(-c4cnc5[nH]ccc5c4)c4c(N)ncnc43)ccc2C1] Intermediate A1-19 | C₄₅H₆₅N₉O₁₁ | [M + H] = 908.49 | [M + H] = 908.3 |
| 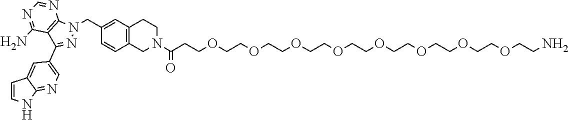 [SMILES: NCCOCCOCCOCCOCCOCCOCCOCCOCCC(=O)N1CCc2cc(Cn3nc(-c4cnc5[nH]ccc5c4)c4c(N)ncnc43)ccc2C1] Intermediate A1-20 | C₄₁H₅₇N₉O₉ | [M + H] = 820.44 | [M + H] = 820.3 |
| 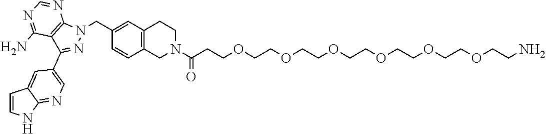 [SMILES: NCCOCCOCCOCCOCCOCCOCCC(=O)N1CCc2cc(Cn3nc(-c4cnc5[nH]ccc5c4)c4c(N)ncnc43)ccc2C1] Intermediate A1-21 | C₃₇H₄₉N₉O₇ | [M + H] = 732.39 | [M + H] = 732.3 |
| 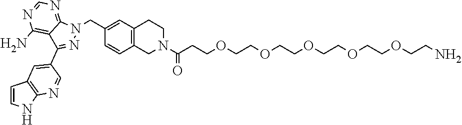 [SMILES: NCCOCCOCCOCCOCCOCCC(=O)N1CCc2cc(Cn3nc(-c4cnc5[nH]ccc5c4)c4c(N)ncnc43)ccc2C1] Intermediate A1-22 | C₃₅H₄₅N₉O₆ | [M + H] = 688.36 | [M + H] = 688.3 |
| 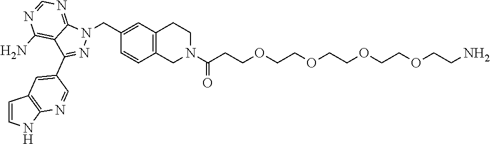 [SMILES: NCCOCCOCCOCCOCCC(=O)N1CCc2cc(Cn3nc(-c4cnc5[nH]ccc5c4)c4c(N)ncnc43)ccc2C1] Intermediate A1-23 | C₃₃H₄₁N₉O₅ | [M + H] = 644.33 | [M + H] = 644.3 |
| 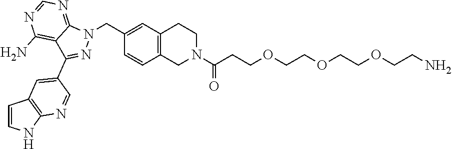 [SMILES: NCCOCCOCCOCCC(=O)N1CCc2cc(Cn3nc(-c4cnc5[nH]ccc5c4)c4c(N)ncnc43)ccc2C1] Intermediate A1-24 | C₃₁H₃₇N₉O₄ | [M + H] = 600.31 | [M + H] = 600.4 |
| 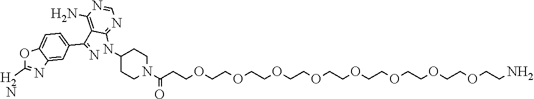 [SMILES: NCCOCCOCCOCCOCCOCCOCCOCCOCCC(=O)N1CCC(n2nc(-c3ccc4oc(N)nc4c3)c3c(N)ncnc32)CC1] Intermediate A1-25 | C₃₆H₅₅N₉O₁₀ | [M + H] = 774.42 | [M + H] = 774.7 |
| 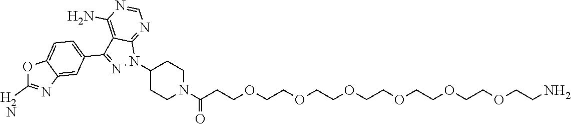 [SMILES: NCCOCCOCCOCCOCCOCCOCCC(=O)N1CCC(n2nc(-c3ccc4oc(N)nc4c3)c3c(N)ncnc32)CC1] Intermediate A1-26 | C₃₂H₄₇N₉O₈ | [M + H] = 686.36 | [M + H] = 686.4 |
| 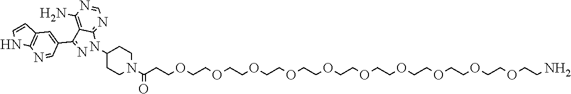 [SMILES: NCCOCCOCCOCCOCCOCCOCCOCCOCCOCCOCCC(=O)N1CCC(n2nc(-c3cnc4[nH]ccc4c3)c3c(N)ncnc32)CC1] Intermediate A1-27 | C₄₀H₆₃N₉O₁₁ | [M + H] = 846.47 | [M + H] = 846.8 |
| 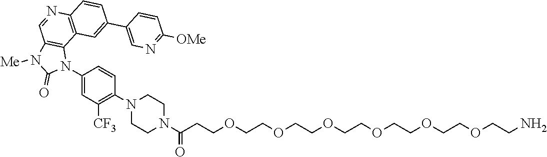 [SMILES: COc1ccc(-c2ccc3ncc4c(c3c2)n(-c2ccc(N3CCN(C(=O)CCOCCOCCOCCOCCOCCOCCN)CC3)c(C(F)(F)F)c2)c(=O)n4C)cn1] Intermediate A1-28 | C₄₃H₅₄F₃N₇O₉ | [M + H] = 870.40 | [M + H] = 870.4 |
| 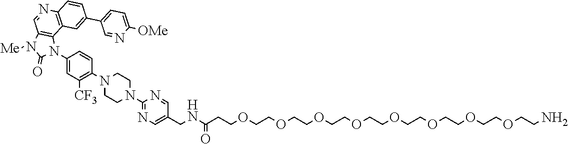 [SMILES: COc1ccc(-c2ccc3ncc4c(c3c2)n(-c2ccc(N3CCN(c5ncc(CNC(=O)CCOCCOCCOCCOCCOCCOCCOCCOCCN)cn5)CC3)c(C(F)(F)F)c2)c(=O)n4C)cn1] Intermediate A1-29 | C₅₂H₆₇F₃N₁₀O₁₁ | [M + H] = 1065.50 | [M + H] = 1065.4 |
| 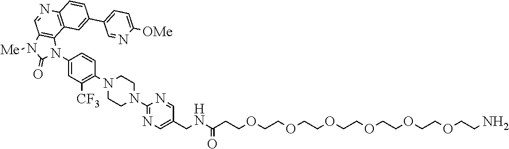 [SMILES: COc1ccc(-c2ccc3ncc4c(c3c2)n(-c2ccc(N3CCN(c5ncc(CNC(=O)CCOCCOCCOCCOCCOCCOCCN)cn5)CC3)c(C(F)(F)F)c2)c(=O)n4C)cn1] Intermediate A1-30 | C₄₈H₅₉F₃N₁₀O₉ | [M + H] = 977.45 | [M + H] = 977.4 |
| 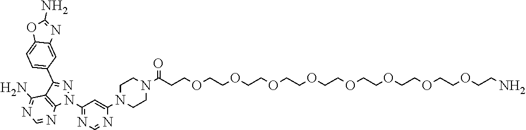 [SMILES: NCCOCCOCCOCCOCCOCCOCCOCCOCCC(=O)N1CCN(c2cc(-n3nc(-c4ccc5oc(N)nc5c4)c4c(N)ncnc43)ncn2)CC1] Intermediate A1-31 | C₃₉H₅₇F₃N₁₂O₁₂ | [M + H] = 853.43 | [M + H] = 853.4 |
| 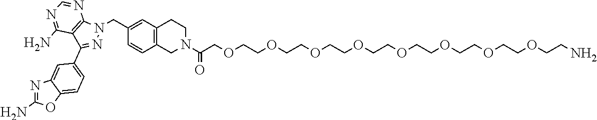 [SMILES: NCCOCCOCCOCCOCCOCCOCCOCCOCC(=O)N1CCc2cc(Cn3nc(-c4ccc5oc(N)nc5c4)c4c(N)ncnc43)ccc2C1] Intermediate A1-32 | C₄₀H₅₅N₉O₁₀ | [M + H] = 822.42 | [M + H] = 822.2 |
| 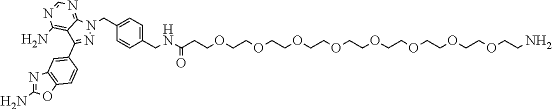 [SMILES: NCCOCCOCCOCCOCCOCCOCCOCCOCCC(=O)NCc1ccc(Cn2nc(-c3ccc4oc(N)nc4c3)c3c(N)ncnc32)cc1] Intermediate A1-33 | C₃₉H₅₅N₉O₁₀ | [M + H] = 810.42 | [M + H] = 810.3 |
| 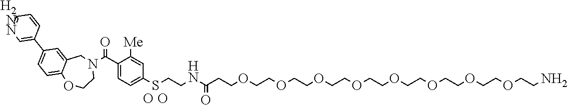 [SMILES: Cc1cc(S(=O)(=O)CCNC(=O)CCOCCOCCOCCOCCOCCOCCOCCOCCN)ccc1C(=O)N1CCOc2ccc(-c3ccc(N)nc3)cc2C1] Intermediate A1-34 | C₄₃H₆₃N₅O₁₃S | [M + H] = 890.42 | [M + H] = 890.3 |
| 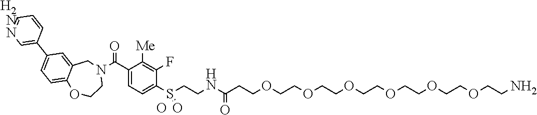 [SMILES: Cc1c(C(=O)N2CCOc3ccc(-c4ccc(N)nc4)cc3C2)ccc(S(=O)(=O)CCNC(=O)CCOCCOCCOCCOCCOCCOCCN)c1F] Intermediate A1-35 | C₃₉H₅₄FN₅O₁₁S | [M + H] = 820.36 | [M + H] = 820.3 |
| 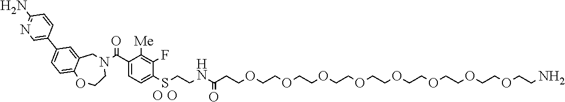 [SMILES: Cc1c(C(=O)N2CCOc3ccc(-c4ccc(N)nc4)cc3C2)ccc(S(=O)(=O)CCNC(=O)CCOCCOCCOCCOCCOCCOCCOCCOCCN)c1F] Intermediate A1-36 | C₄₃H₆₂FN₅O₁₃S | [M + H] = 908.41 | [M + H] = 908.3 |
| 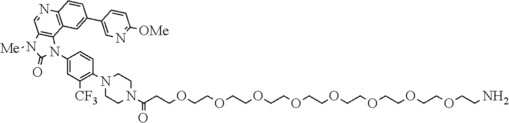 [SMILES: COc1ccc(-c2ccc3ncc4c(c3c2)n(-c2ccc(N3CCN(C(=O)CCOCCOCCOCCOCCOCCOCCOCCOCCN)CC3)c(C(F)(F)F)c2)c(=O)n4C)cn1] Intermediate A1-37 | C₄₇H₆₂F₃N₇O₁₁ | [M + H] = 958.46 | [M + H] = 958.3 |
| 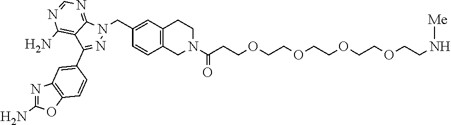 [SMILES: CNCCOCCOCCOCCOCCC(=O)N1CCc2cc(Cn3nc(-c4ccc5oc(N)nc5c4)c4c(N)ncnc43)ccc2C1] Intermediate A1-38 | C₃₄H₄₃N₉O₆ | [M + H] = 674.34 | [M + H] = 674.3 |
| 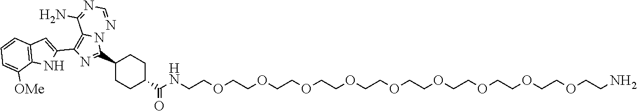 [SMILES: COc1cccc2cc(-c3nc([C@H]4CC[C@H](C(=O)NCCOCCOCCOCCOCCOCCOCCOCCOCCOCCN)CC4)n4ncnc(N)c34)[nH]c12] Intermediate A1-39 | C₄₁H₆₄N₈O₁₁ | [M + H] = 845.48 | [M + H] = 845.3 |
| 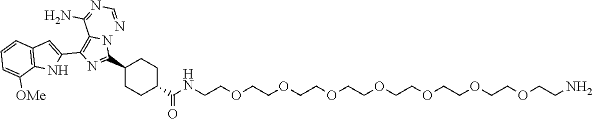 [SMILES: COc1cccc2cc(-c3nc([C@H]4CC[C@H](C(=O)NCCOCCOCCOCCOCCOCCOCCOCCN)CC4)n4ncnc(N)c34)[nH]c12] Intermediate A1-40 | C₃₇H₅₆N₈O₉ | [M + H] = 757.43 | [M + H] = 757.3 |
| 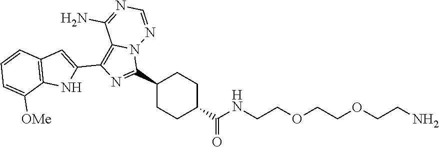 [SMILES: COc1cccc2cc(-c3nc([C@H]4CC[C@H](C(=O)NCCOCCOCCN)CC4)n4ncnc(N)c34)[nH]c12] Intermediate A1-41 | C₂₇H₃₆N₈O₄ | [M + H] = 537.29 | [M + H] = 537.2 |
| 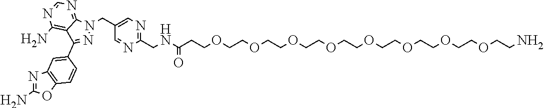 [SMILES: NCCOCCOCCOCCOCCOCCOCCOCCOCCC(=O)NCc1ncc(Cn2nc(-c3ccc4oc(N)nc4c3)c3c(N)ncnc32)cn1] Intermediate A1-42 | C₃₇H₅₃N₁₁O₁₀ | [M + H] = 812.41 | [M + H] = 812.3 |
| 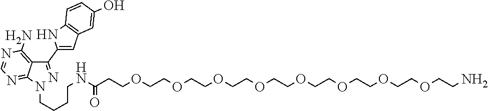 [SMILES: NCCOCCOCCOCCOCCOCCOCCOCCOCCC(=O)NCCCCn1nc(-c2cc3cc(O)ccc3[nH]2)c2c(N)ncnc21] Intermediate A1-43 | C₃₆H₅₆N₈O₁₀ | [M + H] = 761.42 | [M + H] = 761.3 |
| 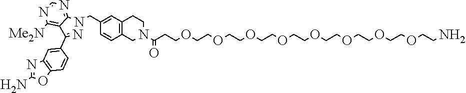 [SMILES: CN(C)c1ncnc2c1c(-c1ccc3oc(N)nc3c1)nn2Cc1ccc2c(c1)CCN(C(=O)CCOCCOCCOCCOCCOCCOCCOCCOCCN)C2] Intermediate A1-44 | C₄₃H₆₁N₉O₁₀ | [M + H] = 864.46 | [M + H] = 864.4 |
| 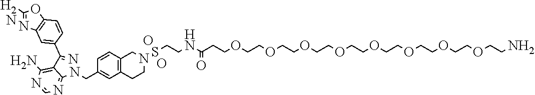 [SMILES: NCCOCCOCCOCCOCCOCCOCCOCCOCCC(=O)NCCS(=O)(=O)N1CCc2cc(Cn3nc(-c4ccc5oc(N)nc5c4)c4c(N)ncnc43)ccc2C1] Intermediate A1-45 | C₄₃H₆₂N₁₀O₁₂S | [M + H] = 943.44 | [M + H] = 943.3 |
| 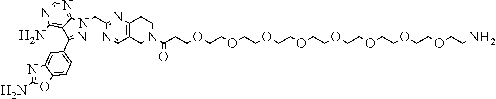 [SMILES: NCCOCCOCCOCCOCCOCCOCCOCCOCCC(=O)N1CCc2nc(Cn3nc(-c4ccc5oc(N)nc5c4)c4c(N)ncnc43)ncc2C1] Intermediate A1-46 | C₃₉H₅₅N₁₁O₁₀ | [M + H] = 838.42 | [M + H] = 838.3 |

Following General Procedure 1, but using the appropriate Intermediate A1 in Table 5 and PEG carboxylic acid, the Intermediates A2 in Table 6 were prepared:

TABLE 6
Additional amines prepared

| | Molecular | Calculated | Observed |
|---|---|---|---|
| Structure | Formula | MW | MW |
| 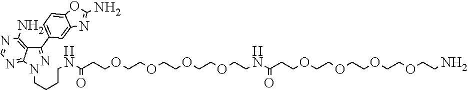 [SMILES: NCCOCCOCCOCCOCCC(=O)NCCOCCOCCOCCOCCC(=O)NCCCCn1nc(-c2ccc3oc(N)nc3c2)c2c(N)ncnc21] Intermediate A2-1 | C₃₆H₆₀N₁₀O₁₁ | [M + H] = 833.45 | [M + H] = 833.8 |
| 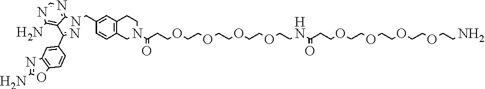 [SMILES: NCCOCCOCCOCCOCCC(=O)NCCOCCOCCOCCOCCC(=O)N1CCc2cc(Cn3nc(-c4ccc5oc(N)nc5c4)c4c(N)ncnc43)ccc2C1] Intermediate A2-2 | C₄₄H₆₂N₁₀O₁₁ | [M + H] = 907.47 | [M + H] = 908.0 |
| 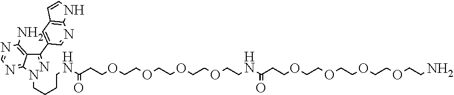 [SMILES: NCCOCCOCCOCCOCCC(=O)NCCOCCOCCOCCOCCC(=O)NCCCCn1nc(-c2cnc3[nH]ccc3c2)c2c(N)ncnc21] Intermediate A2-3 | C₃₈H₆₀N₁₀O₁₀ | [M + H] = 817.46 | [M + H] = 817.4 |
| 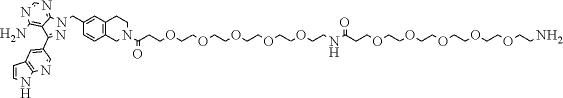 [SMILES: NCCOCCOCCOCCOCCOCCC(=O)NCCOCCOCCOCCOCCOCCC(=O)N1CCc2cc(Cn3nc(-c4cnc5[nH]ccc5c4)c4c(N)ncnc43)ccc2C1] Intermediate A2-4 | C₄₈H₇₀N₁₀O₁₂ | [M + H] = 979.53 | [M + H] = 979.9 |
| 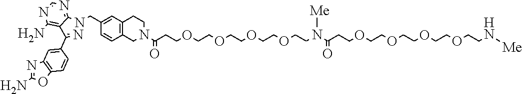 [SMILES: CNCCOCCOCCOCCOCCC(=O)N(C)CCOCCOCCOCCOCCC(=O)N1CCc2cc(Cn3nc(-c4ccc5oc(N)nc5c4)c4c(N)ncnc43)ccc2C1] Intermediate A2-5 | C₄₆H₆₆N₁₀O₁₁ | [M + H] = 935.50 | [M + H] = 935.3 |
| 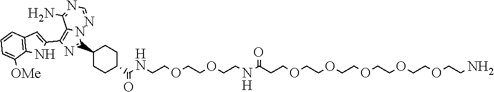 [SMILES: COc1cccc2cc(-c3nc([C@H]4CC[C@H](C(=O)NCCOCCOCCNC(=O)CCOCCOCCOCCOCCOCCN)CC4)n4ncnc(N)c34)[nH]c12] Intermediate A2-6 | C₄₀H₆₁N₉O₁₀ | [M + H] = 828.46 | [M + H] = 828.3 |

### General Procedure 2: Coupling of a 4-Nitrophenyl Carbonate Containing Rapamycin Monomer and an Active Site Inhibitor Containing Intermediate Having a Primary or Secondary Amine

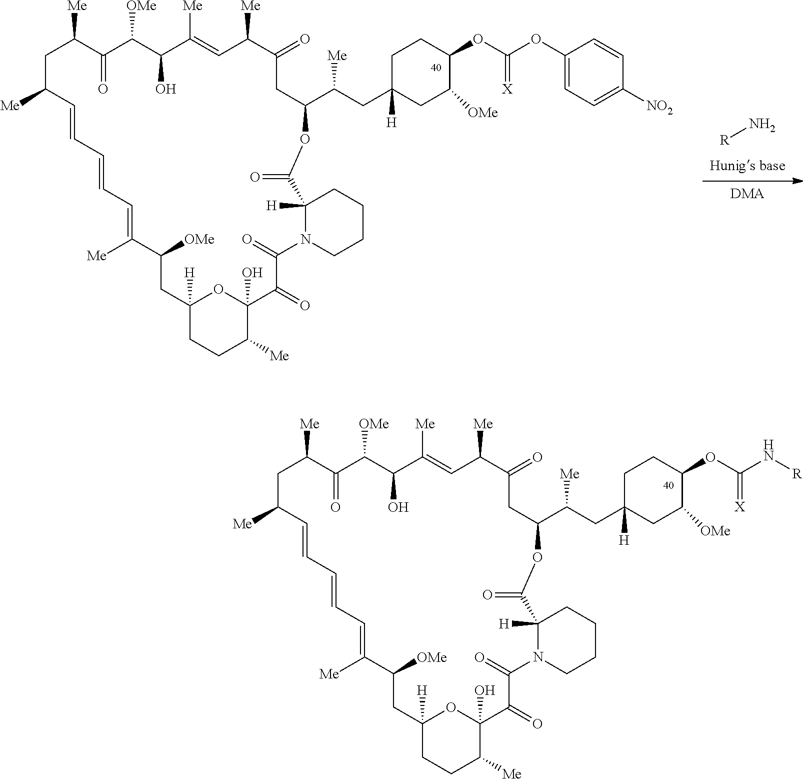

To a 0.02 M solution of 4-nitrophenyl carbonate containing rapamycin monomer (1.0 equiv) and an active site inhibitor containing intermediate (2.0 equiv) in DMA was added DIPEA (4.0 equiv). The resulting solution was stirred at room temperature under nitrogen. Upon completion as determined by LCMS analysis, the crude reaction mixture was purified by preparative HPLC to provide the product.

### Example 2: Synthesis of Series 1 Bivalent Rapamycin Compound

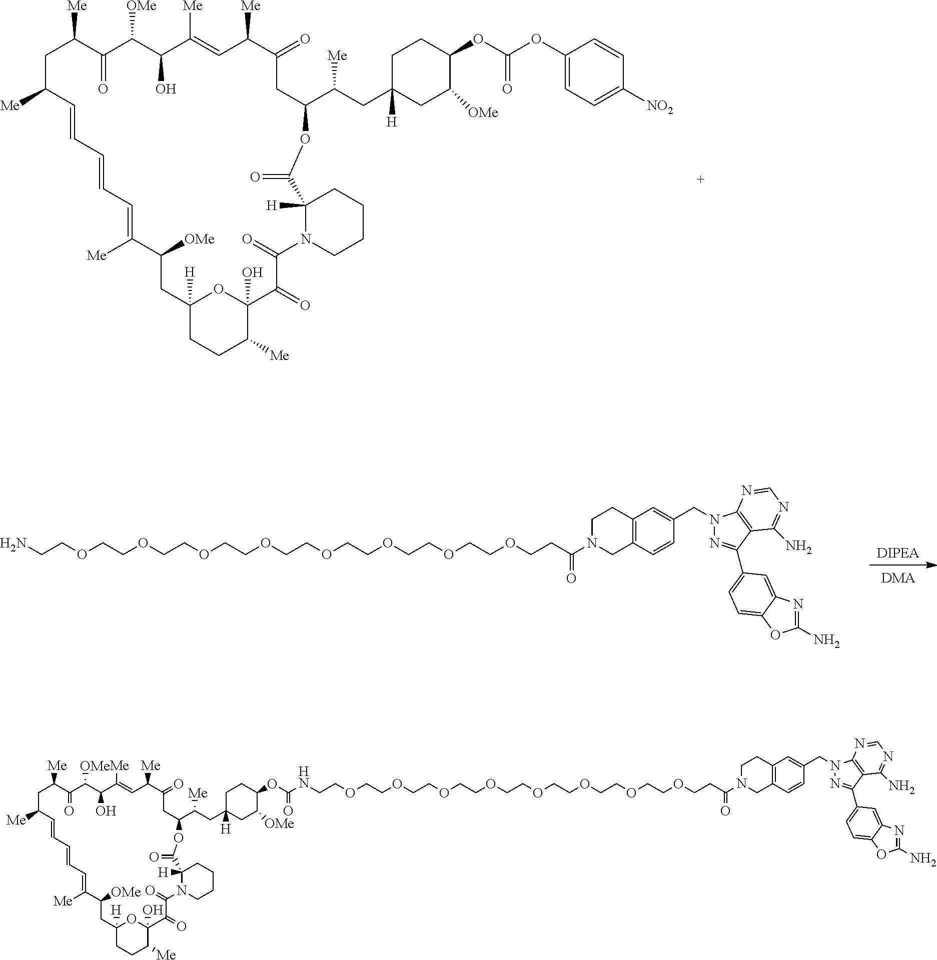

To a solution of 40(R)—O-(4-nitrophenyl carbonate) rapamycin (25 mg, 23.16 μmol, 1.0 equiv) and Intermediate A1-7 (42.0 mg, 46.32 μmol, 2.0 equiv) in DMA (1.15 mL) was added DIPEA (16.0 μL, 92.64 μmol, 4.0 equiv). The reaction was stirred for 18 h, at which point the reaction mixture purified by reverse phase chromatography (10→40→95% MeCN+0.1% formic acid/H₂O+0.1% formic acid) to give the product (9.92 mg, 24% yield) as a white solid. LCMS (ESI) m/z: [M+H] calcd for C₉₃H₁₃₄N₁₀O₂₄: 1775.97; found 1775.7.

Following General Procedure 2, but using the appropriate 4-nitrophenyl carbonate containing rapamycin monomer in Table 1 and Intermediates A 1 and A2 from Tables 5 and 6, the Series 1 bivalent analogs in Table 7 were synthesized:

TABLE 7
Series 1 Bivalent Compounds:

| | Molecular | Calculated | Observed |
|---|---|---|---|
| Structure | Formula | MW | MW |
| 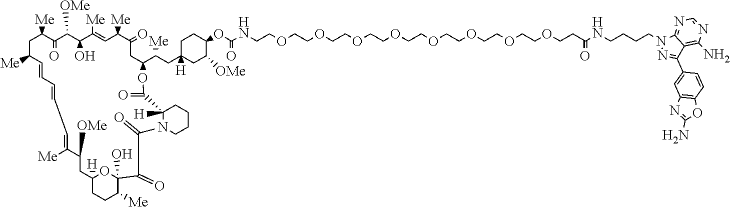 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4ccc5oc(N)nc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] Example 1 | C₈₇H₁₃₂N₁₀O₂₄ | [M + H] = 1701.95 | [M + H] = 1701.8 |
| 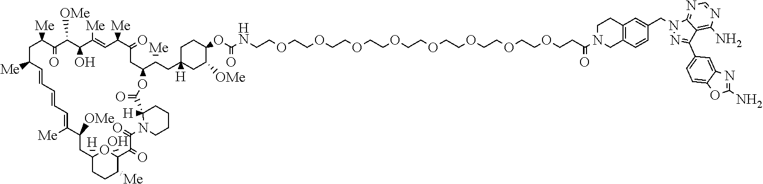 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 2 | C₉₃H₁₃₄N₁₀O₂₄ | [M + H] = 1775.97 | [M + H] = 1775.7 |
| 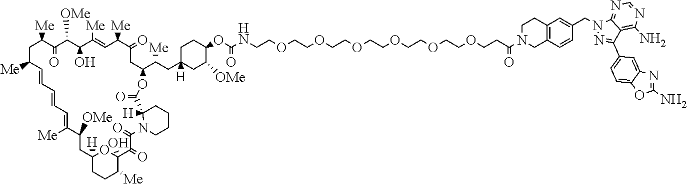 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 3 | C₈₉H₁₂₆N₁₀O₂₂ | [M + H] = 1687.91 | [M + H] = 1687.8 |
| 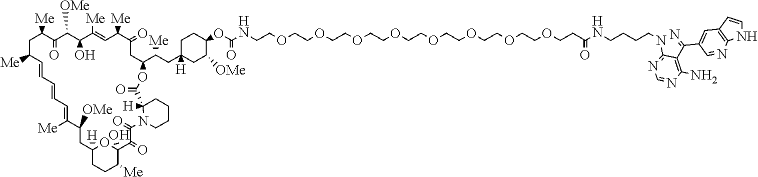 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4cnc5[nH]ccc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] Example 4 | C₈₇H₁₃₂N₁₀O₂₃ | [M + H] = 1684.95 | [M + H] = 1685.1 |
| 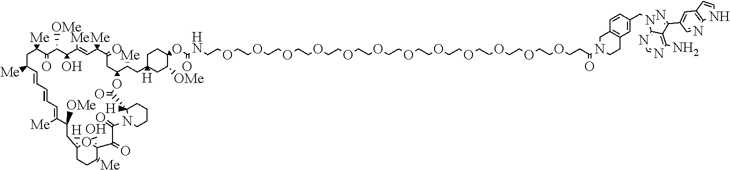 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 5 | C₁₀₁H₁₅₀N₁₀O₂₇ | [M + H] = 1936.08 | [M + H] = 1936.4 |
| 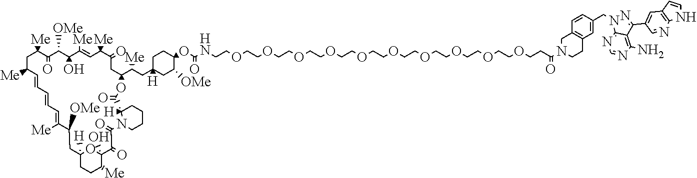 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 6 | C₉₇H₁₄₂N₁₀O₂₅ | [M + H] = 1848.02 | [M + H] = 1848.3 |
| 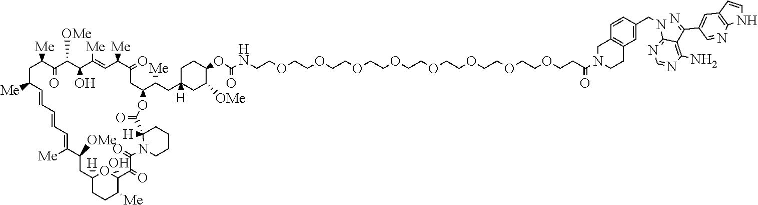 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 7 | C₉₃H₁₃₄N₁₀O₂₃ | [M + H] = 1759.97 | [M + H] = 1760.1 |
| 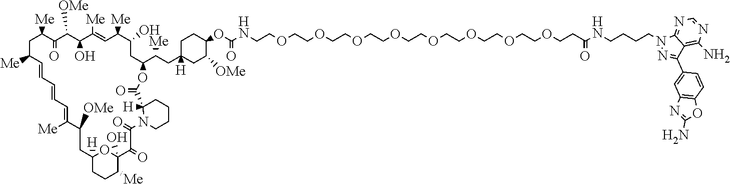 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4ccc5oc(N)nc5c4)c4c(N)ncnc43)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 8 | C₈₇H₁₃₄N₁₀O₂₄ | [M + H] = 1703.97 | [M + H] = 1703.7 |
| 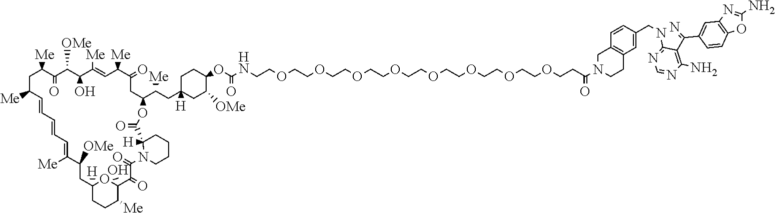 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 9 | C₉₃H₁₃₆N₁₀O₂₄ | [M + H] = 1777.98 | [M + H] = 1777.7 |
| 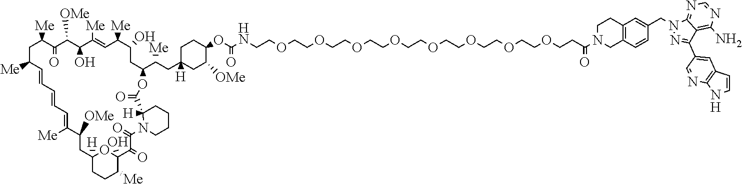 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 10 | C₉₃H₁₃₆N₁₀O₂₃ | [M + H] = 1761.99 | [M + H] = 1761.7 |
| 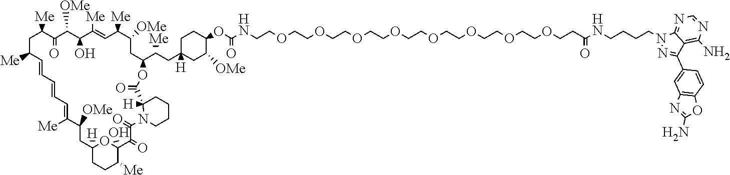 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4ccc5oc(N)nc5c4)c4c(N)ncnc43)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 11 | C₈₈H₁₃₆N₁₀O₂₄ | [M + H] = 1717.98 | [M + H] = 1718.0 |
| 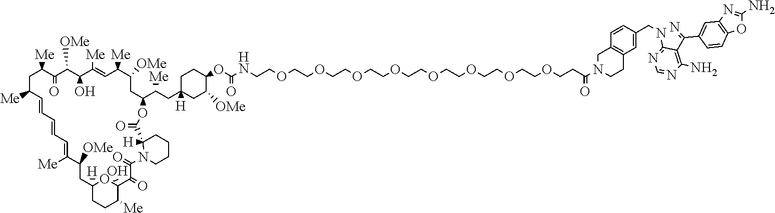 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 12 | C₉₄H₁₃₈N₁₀O₂₄ | [M + H] = 1792.00 | [M + H] = 1791.9 |
| 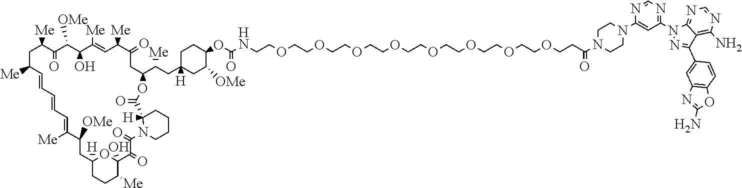 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCN(c4cc(-n5nc(-c6ccc7oc(N)nc7c6)c6c(N)ncnc65)ncn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 13 | C₉₁H₁₃₃N₁₃O₂₄ | [M + H] = 1792.97 | [M + H] = 1793.0 |
| 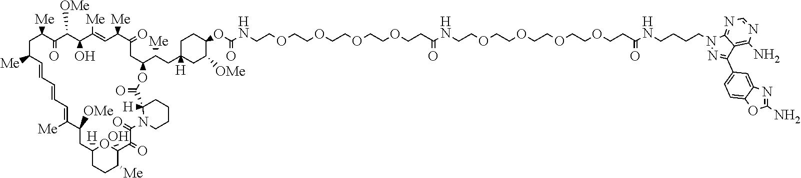 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)NCCCCn3nc(-c4ccc5oc(N)nc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] Example 14 | C₉₀H₁₃₇N₁₁O₂₅ | [M + H] = 1772.99 | [M + H] = 1772.9 |
| 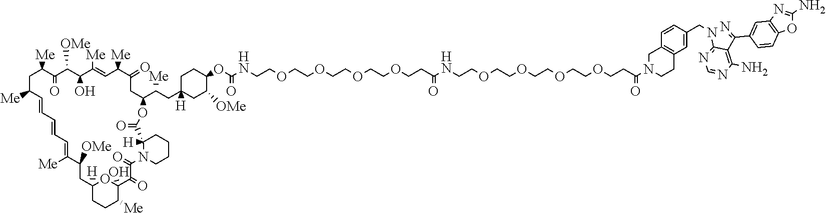 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 15 | C₉₆H₁₃₉N₁₁O₂₅ | [M + H] = 1847.00 | [M + H] = 1847.0 |
| 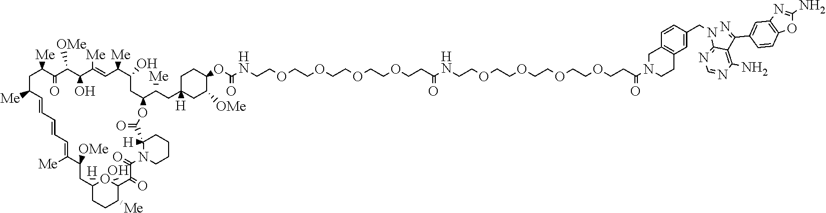 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 16 | C₉₆H₁₄₁N₁₁O₂₅ | [M + H] = 1849.02 | [M + H] = 1848.9 |
| 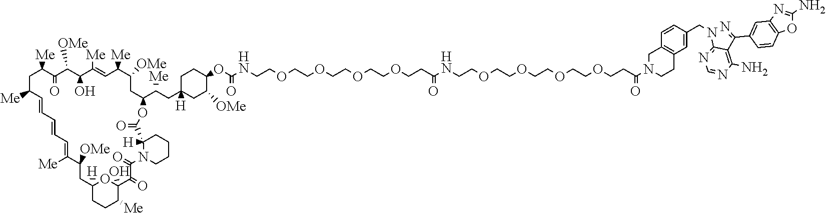 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 17 | C₉₇H₁₄₃N₁₁O₂₅ | [M + H] = 1863.04 | [M + H] = 1863.0 |
| 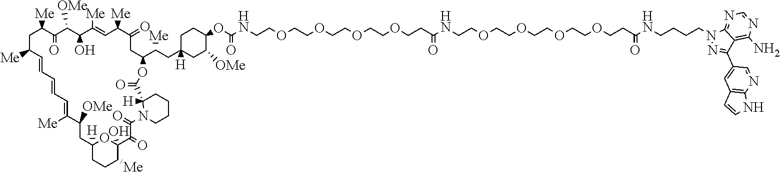 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)NCCCCn3nc(-c4cnc5[nH]ccc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] Example 18 | C₉₀H₁₃₇N₁₁O₂₄ | [M + H] = 1756.99 | [M + H] = 1756.8 |
| 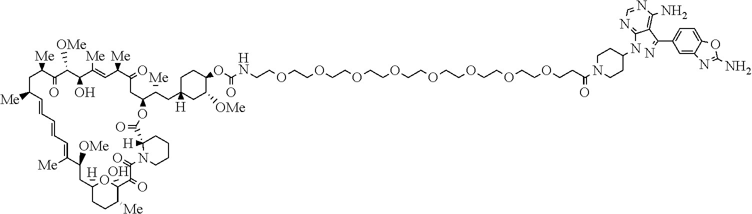 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCC(n4nc(-c5ccc6oc(N)nc6c5)c5c(N)ncnc54)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 19 | C₈₈H₁₃₂N₁₀O₂₄ | [M + H] = 1713.95 | [M + H] = 1713.9 |
| 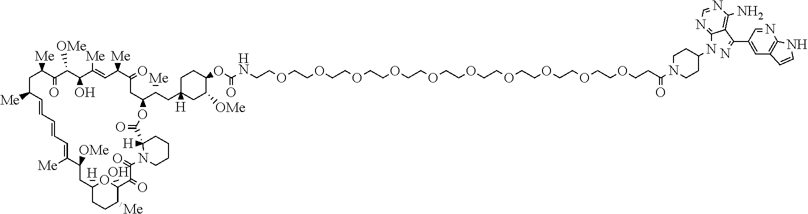 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCC(n4nc(-c5cnc6[nH]ccc6c5)c5c(N)ncnc54)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 20 | C₉₂H₁₄₀N₁₀O₂₅ | [M + H] = 1786.01 | [M + H] = 1786.0 |
| 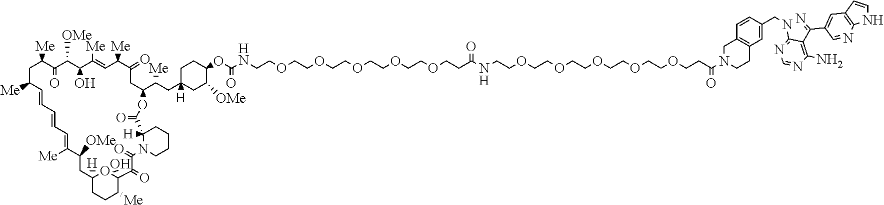 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCC(=O)NCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 21 | C₁₀₀H₁₄₇N₁₁O₂₆ | [M + H] = 1919.06 | [M + H] = 1919.0 |
| 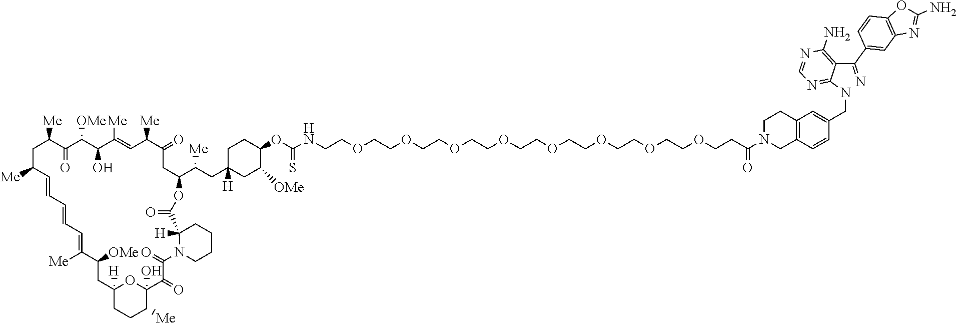 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 22 | C₉₃H₁₃₄N₁₀O₂₃S | [M + H] = 1791.94 | [M + H] = 1791.8 |
| 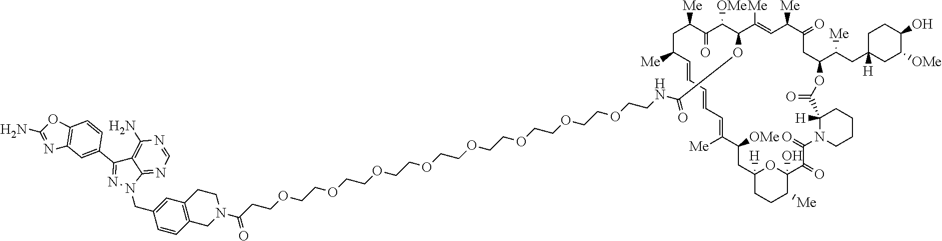 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@@H](OC)C(=O)[C@H](C)C[C@H](C)/C=C/C=C/C=C/1C] Example 23 | C₉₃H₁₃₄N₁₀O₂₄ | [M + H] = 1775.97 | [M + H] = 1775.9 |
| 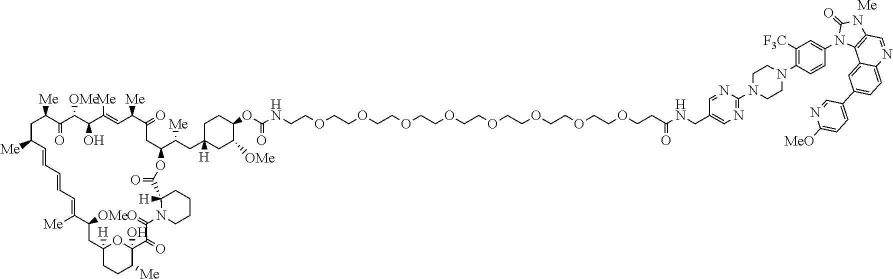 [SMILES: COc1ccc(-c2ccc3ncc4c(c3c2)n(-c2ccc(N3CCN(c5ncc(CNC(=O)CCOCCOCCOCCOCCOCCOCCOCCOCCNC(=O)O[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1] Example 24 | C₁₀₄H₁₄₄F₃N₁₁O₂₅ | [M + 2H]/2 = 1003.03 | [M + 2H]/2 = 1003.5 |
| 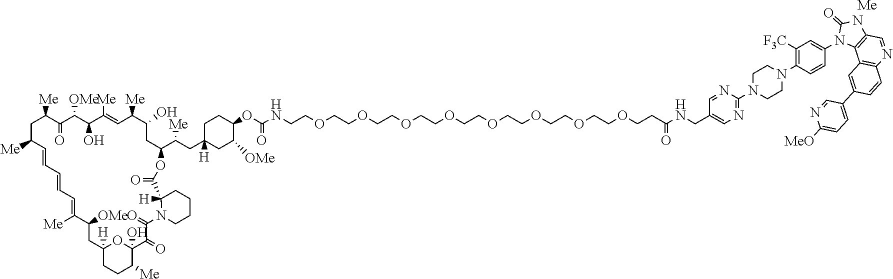 [SMILES: COc1ccc(-c2ccc3ncc4c(c3c2)n(-c2ccc(N3CCN(c5ncc(CNC(=O)CCOCCOCCOCCOCCOCCOCCOCCOCCNC(=O)O[C@@H]6CC[C@@H](C[C@@H](C)[C@@H]7C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1] Example 25 | C₁₀₄H₁₄₆F₃N₁₁O₂₅ | [M + 2H]/2 = 1004.03 | [M + 2H]/2 = 1004.5 |
| 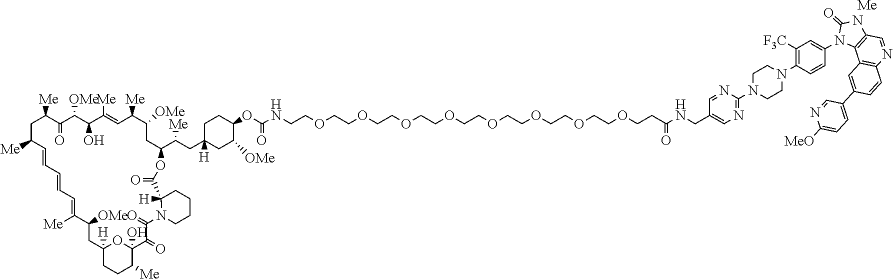 [SMILES: COc1ccc(-c2ccc3ncc4c(c3c2)n(-c2ccc(N3CCN(c5ncc(CNC(=O)CCOCCOCCOCCOCCOCCOCCOCCOCCNC(=O)O[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1] Example 26 | C₁₀₅H₁₄₈F₃N₁₁O₂₅ | [M + 2H]/2 = 1011.04 | [M + 2H]/2 = 1011.5 |
| 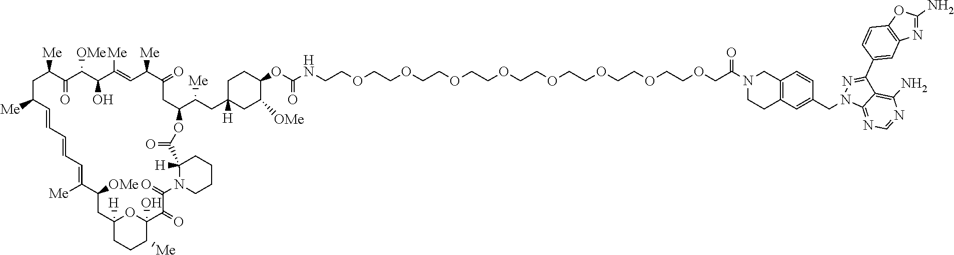 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 71 | C₉₂H₁₃₂N₁₀O₂₄ | [M + H] = 1761.95 | [M + H] = 1761.8 |
| 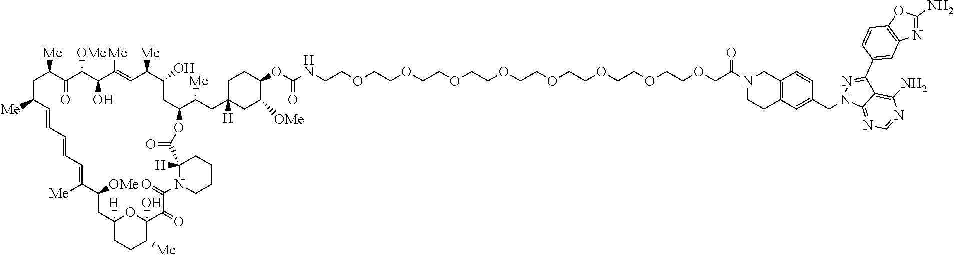 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 72 | C₉₂H₁₃₄N₁₀O₂₄ | [M + H] = 1763.97 | [M + H] = 1763.8 |
| 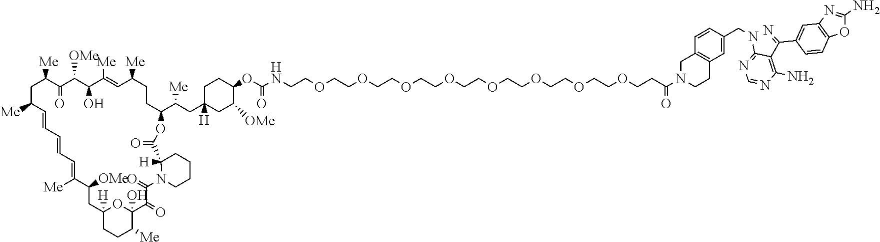 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC[C@H](C)/C=C(\C)[C@@H](O)[C@@H](OC)C(=O)[C@H](C)C[C@H](C)/C=C/C=C/C=C/1C] Example 73 | C₉₃H₁₃₆N₁₀O₂₃ | [M + H] = 1761.99 | [M + H] = 1762.0 |
| 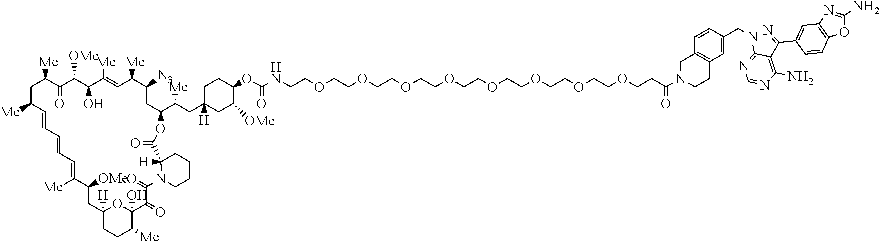 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] Example 74 | C₉₃H₁₃₅N₁₃O₂₃ | [M + H] = 1802.99 | [M + H] = 1802.9 |
| 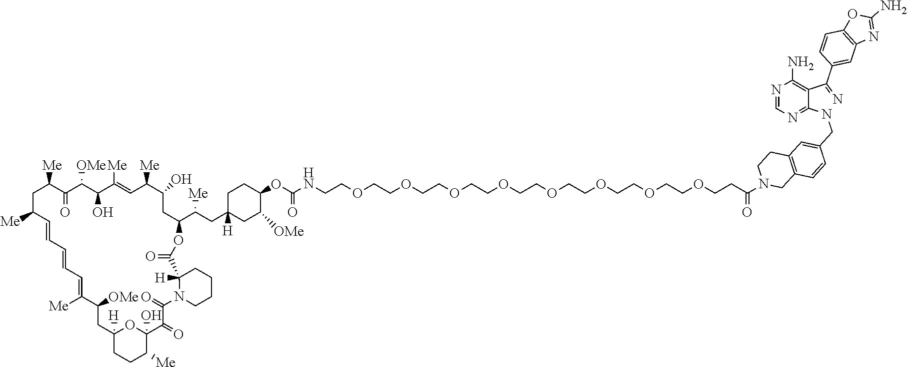 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 75 | C₉₄H₁₃₈N₁₀O₂₄ | [M + H] = 1792.00 | [M + H] = 1791.7 |
| 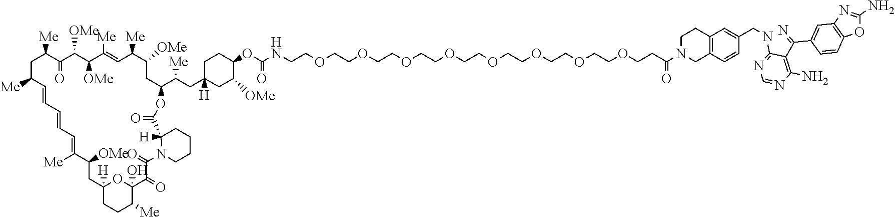 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](OC)[C@@H](OC)C(=O)[C@H](C)C[C@H](C)/C=C/C=C/C=C/1C] Example 76 | C₉₅H₁₄₀N₁₀O₂₄ | [M + H] = 1806.01 | [M + H] = 1805.8 |
| 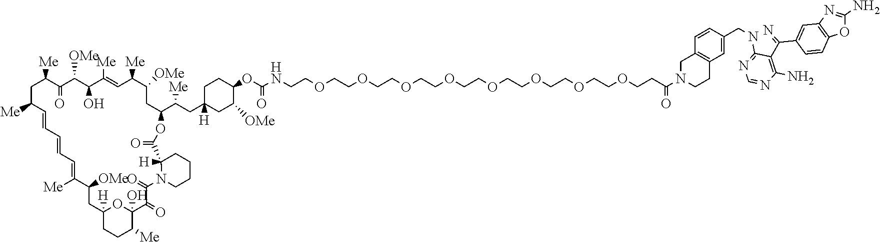 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 77 | C₉₃H₁₃₆N₁₀O₂₃S | [M + H] = 1793.96 | [M + H] = 1793.9 |
| 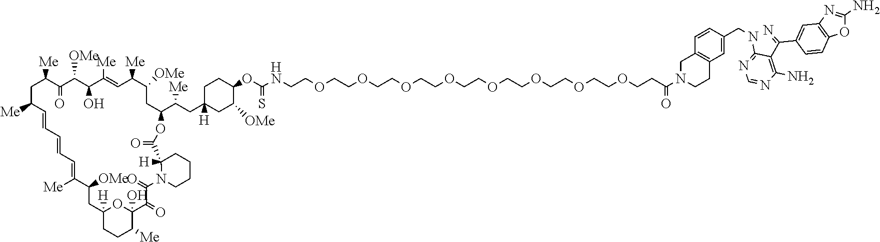 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 78 | C₉₄H₁₃₈N₁₀O₂₃S | [M + H] = 1807.97 | [M + H] = 1807.9 |
| 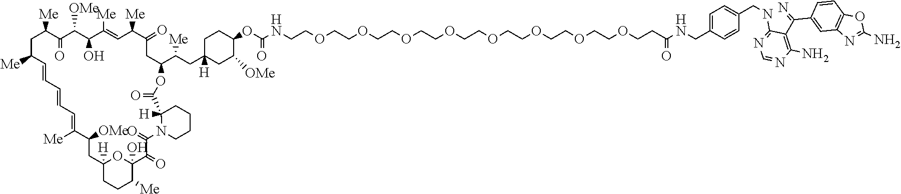 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 79 | C₉₁H₁₃₂N₁₀O₂₄ | [M + H] = 1749.95 | [M + H] = 1749.9 |
| 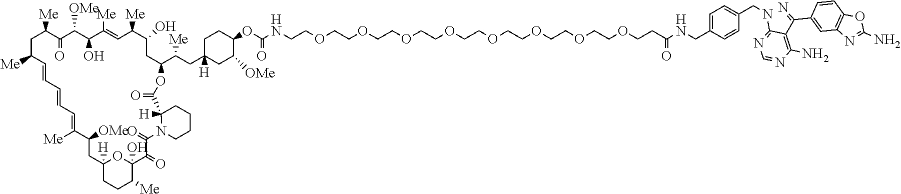 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 80 | C₉₁H₁₃₄N₁₀O₂₄ | [M + H] = 1751.97 | [M + H] = 1751.9 |
| 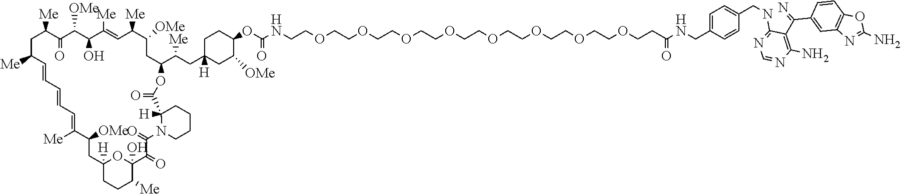 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 81 | C₉₂H₁₃₆N₁₀O₂₄ | [M + H] = 1765.98 | [M + H] = 1765.8 |
| 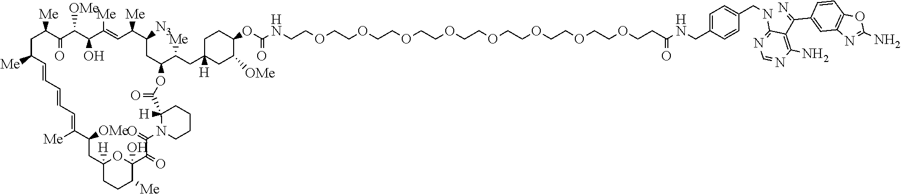 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] Example 82 | C₉₁H₁₃₃N₁₃O₂₃ | [M + H] = 1776.97 | [M + H] = 1776.9 |
| 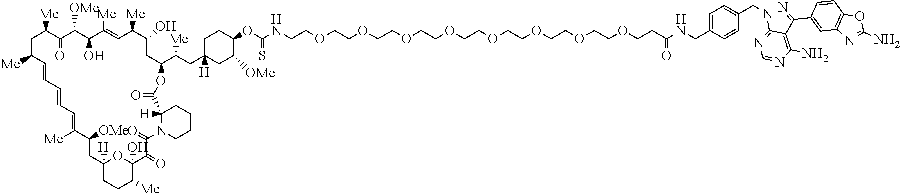 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 83 | C₉₁H₁₃₃N₁₀O₂₃S | [M + H] = 1766.93 | [M + H] = 1766.8 |
| 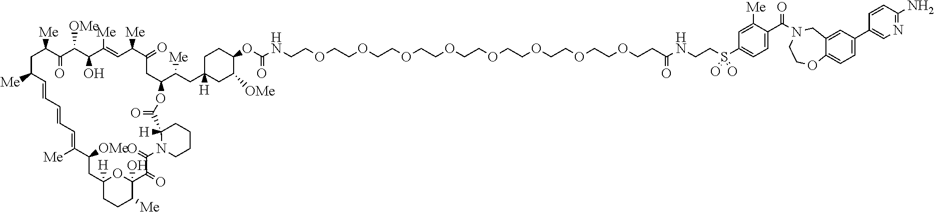 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 84 | C₉₅H₁₄₀N₆O₂₇S | [M + H] = 1829.96 | [M + H] = 1830.0 |
| 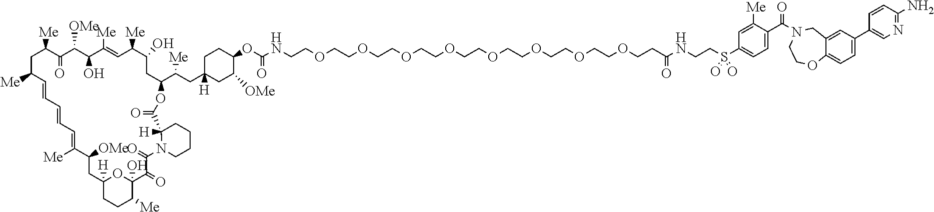 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 85 | C₉₅H₁₄₂N₆O₂₇S | [M + H] = 1831.97 | [M + H] = 1831.9 |
| 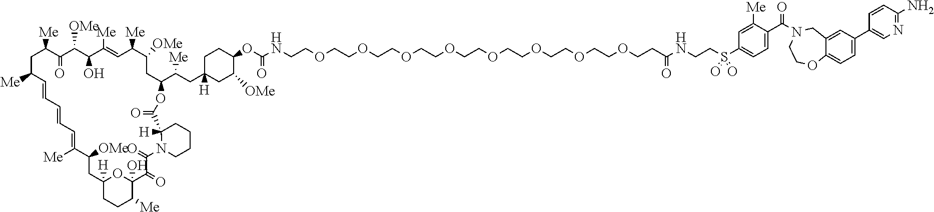 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 86 | C₉₆H₁₄₄N₆O₂₇S | [M + H] = 1845.99 | [M + H] = 1846.0 |
| 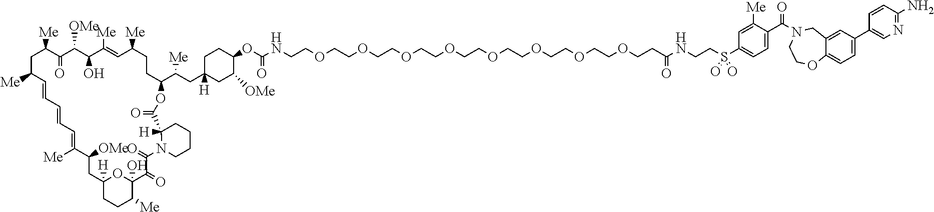 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)CC[C@H](C)/C=C(\C)[C@@H](O)[C@@H](OC)C(=O)[C@H](C)C[C@H](C)/C=C/C=C/C=C/1C] Example 87 | C₉₅H₁₄₂N₆O₂₆S | [M + H] = 1815.98 | [M + H] = 1815.8 |
| 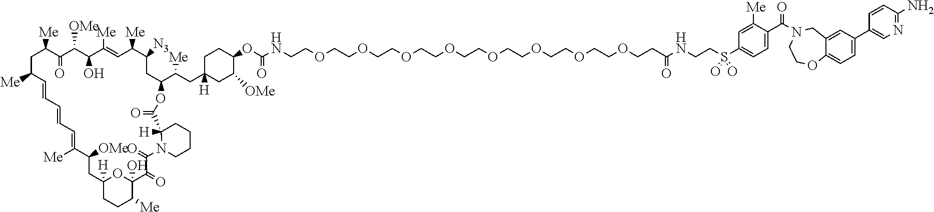 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] Example 88 | C₉₅H₁₄₁N₉O₂₆S | [M + H] = 1856.98 | [M + H] = 1856.8 |
| 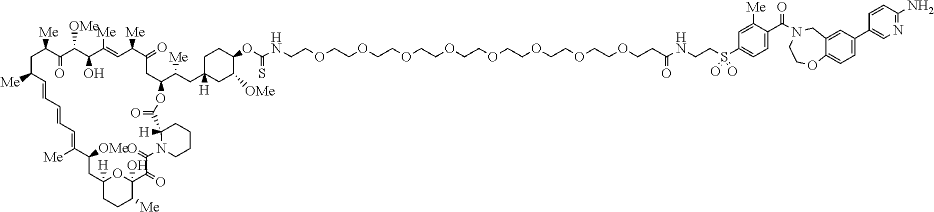 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 89 | C₉₅H₁₄₀N₆O₂₆S₂ | [M + H] = 1845.93 | [M + H] = 1846.0 |
| 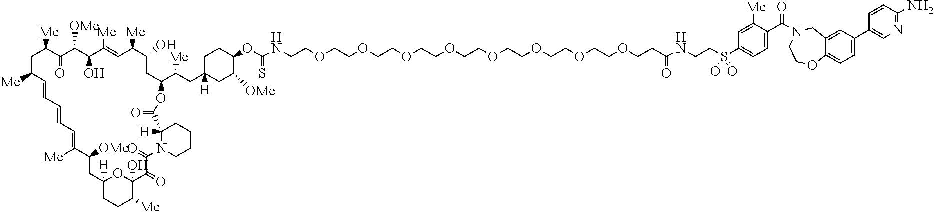 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 90 | C₉₅H₁₄₂N₆O₂₆S₂ | [M + H] = 1847.95 | [M + H] = 1847.9 |
| 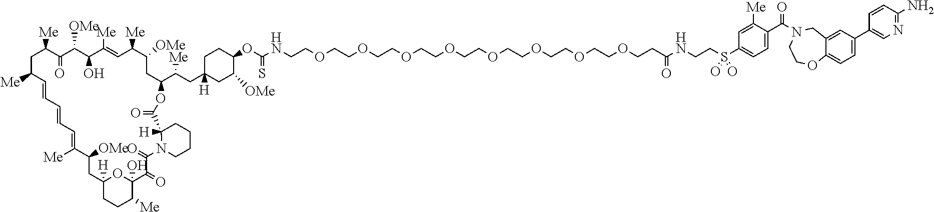 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 91 | C₉₆H₁₄₄N₆O₂₆S₂ | [M + H] = 1861.96 | [M + H] = 1861.7 |
| 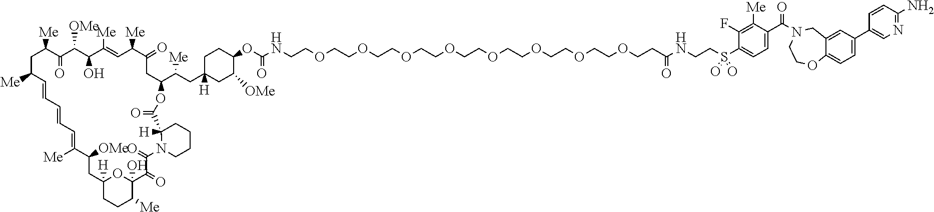 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)CC(=O)[C@H](C)/C=C(\C)[C@@H](O)[C@@H](OC)C(=O)[C@H](C)C[C@H](C)/C=C/C=C/C=C/1C] Example 92 | C₉₅H₁₃₉FN₆O₂₇S | [M + H] = 1847.95 | [M + H] = 1848.0 |
| 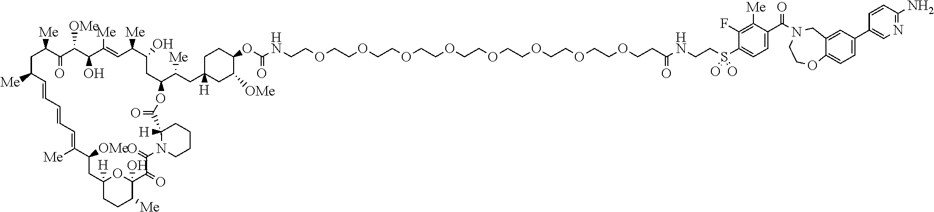 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 93 | C₉₅H₁₄₁FN₆O₂₇S | [M + H] = 1849.96 | [M + H] = 1850.0 |
| 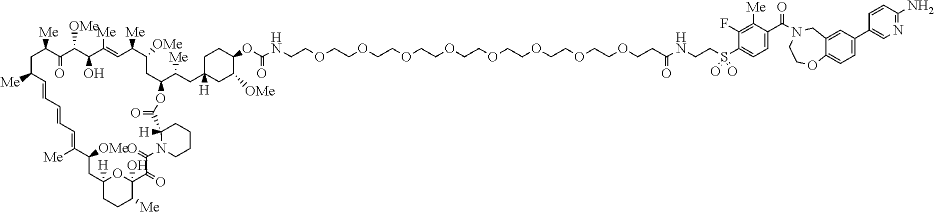 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 94 | C₉₆H₁₄₃FN₆O₂₇S | [M + H] = 1863.98 | [M + H] = 1864.0 |
| 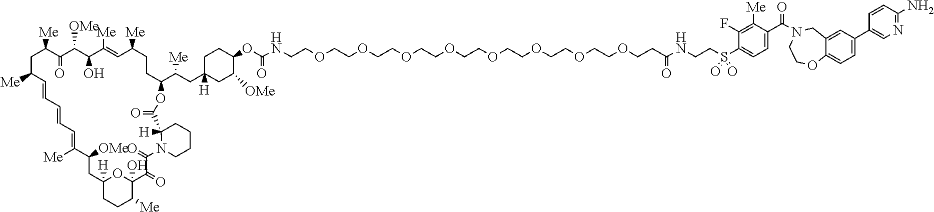 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)CC[C@H](C)/C=C(\C)[C@@H](O)[C@@H](OC)C(=O)[C@H](C)C[C@H](C)/C=C/C=C/C=C/1C] Example 95 | C₉₅H₁₄₁FN₆O₂₆S | [M + H] = 1833.97 | [M + H] = 1833.9 |
| 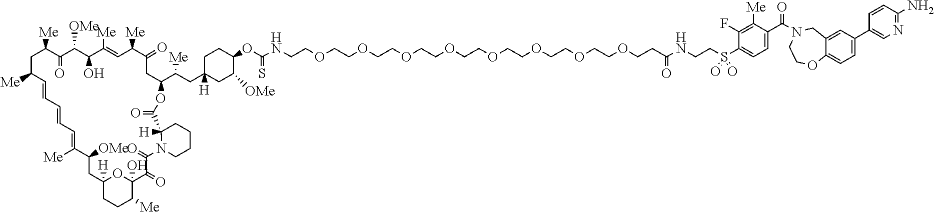 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)CC(=O)[C@H](C)/C=C(\C)[C@@H](O)[C@@H](OC)C(=O)[C@H](C)C[C@H](C)/C=C/C=C/C=C/1C] Example 96 | C₉₅H₁₃₉FN₆O₂₆S₂ | [M + H] = 1863.92 | [M + H] = 1863.8 |
| 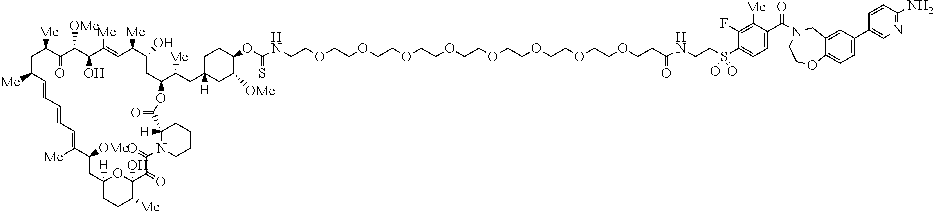 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 97 | C₉₅H₁₄₁FN₆O₂₆S₂ | [M + H] = 1865.94 | [M + H] = 1865.8 |
| 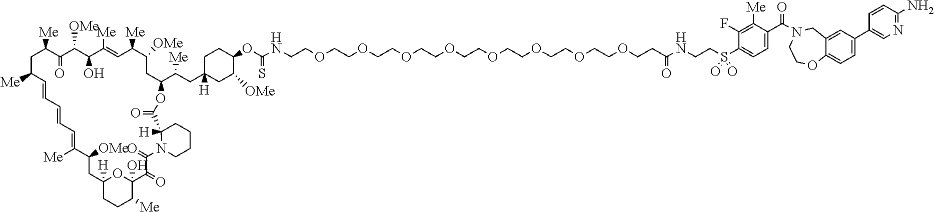 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 98 | C₉₆H₁₄₃FN₆O₂₆S₂ | [M + H] = 1879.96 | [M + H] = 1879.9 |
| 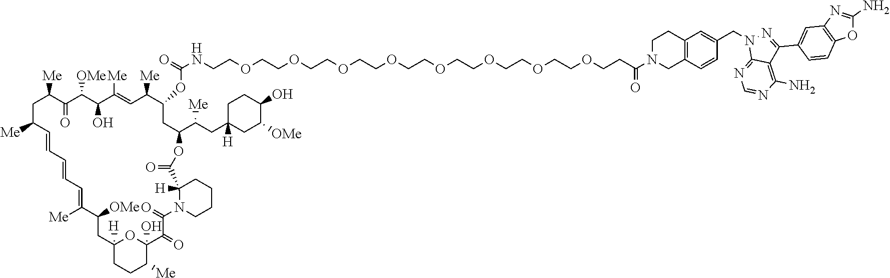 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@H](C)/C=C(\C)[C@@H](O)[C@@H](OC)C(=O)[C@H](C)C[C@H](C)/C=C/C=C/C=C/1C] Example 99 | C₉₃H₁₃₆N₁₀O₂₄ | [M + H] = 1777.98 | [M + H] = 1777.8 |
| 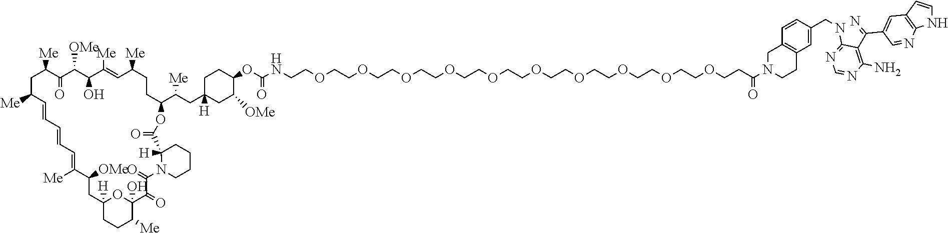 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC[C@H](C)/C=C(\C)[C@@H](O)[C@@H](OC)C(=O)[C@H](C)C[C@H](C)/C=C/C=C/C=C/1C] Example 100 | C₉₇H₁₄₄N₁₀O₂₄ | [M + H] = 1834.04 | [M + H] = 1833.8 |
| 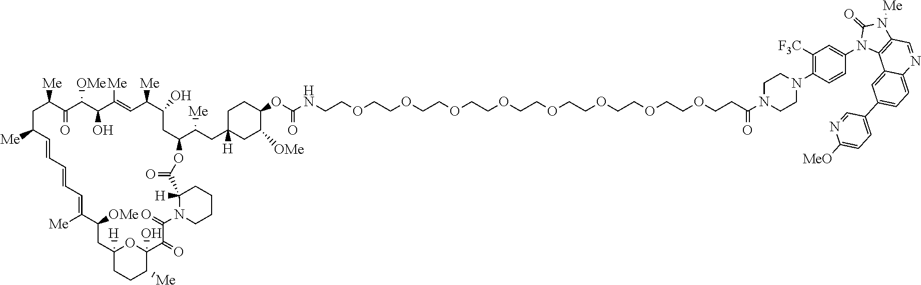 [SMILES: COc1ccc(-c2ccc3ncc4c(c3c2)n(-c2ccc(N3CCN(C(=O)CCOCCOCCOCCOCCOCCOCCOCCOCCNC(=O)O[C@@H]5CC[C@@H](C[C@@H](C)[C@@H]6C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC3)c(C(F)(F)F)c2)c(=O)n4C)cn1] Example 101 | C₉₉H₁₄₁F₃N₈O₂₅ | [M + H] = 1900.00 | [M + H] = 1899.9 |
| 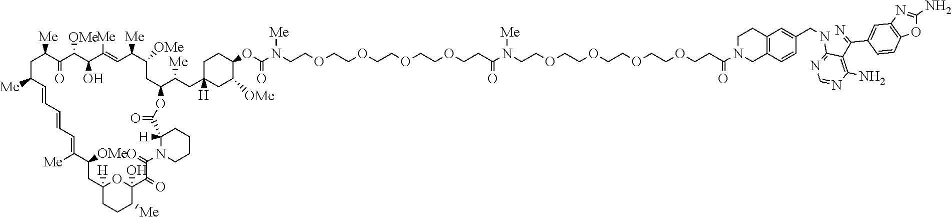 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)CCOCCOCCOCCOCCC(=O)N(C)CCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 102 | C₉₉H₁₄₇N₁₁O₂₅ | [M + H] = 1891.06 | [M + H] = 1890.9 |
| 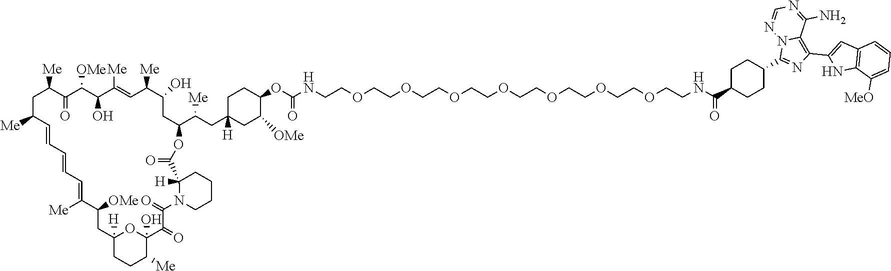 [SMILES: COc1cccc2cc(-c3nc([C@H]4CC[C@H](C(=O)NCCOCCOCCOCCOCCOCCOCCOCCNC(=O)O[C@@H]5CC[C@@H](C[C@@H](C)[C@@H]6C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] Example 136 | C₈₉H₁₃₅N₉O₂₃ | [M + H] = 1698.97 | [M + H] = 1698.8 |
| 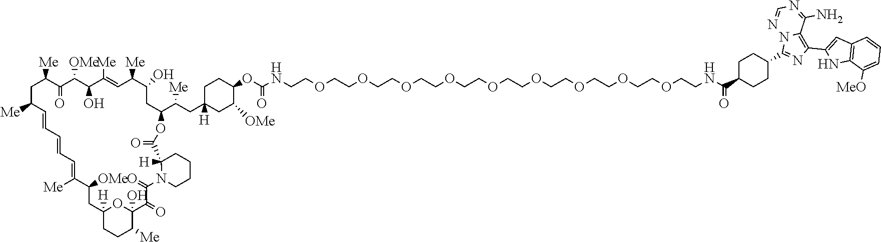 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] Example 137 | C₉₃H₁₄₃N₉O₂₅ | [M + H] = 1787.03 | [M + H] = 1786.7 |
| 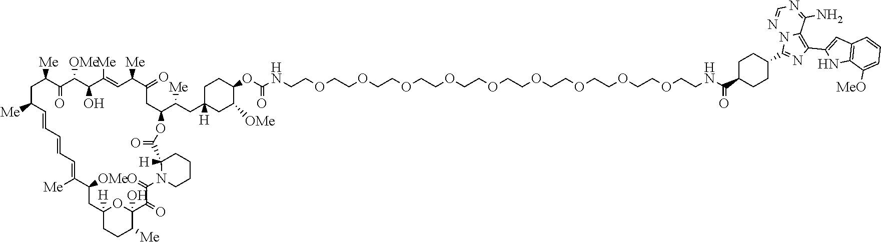 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] Example 138 | C₉₃H₁₄₁N₉O₂₅ | [M + H] = 1785.01 | [M + H] = 1784.9 |
| 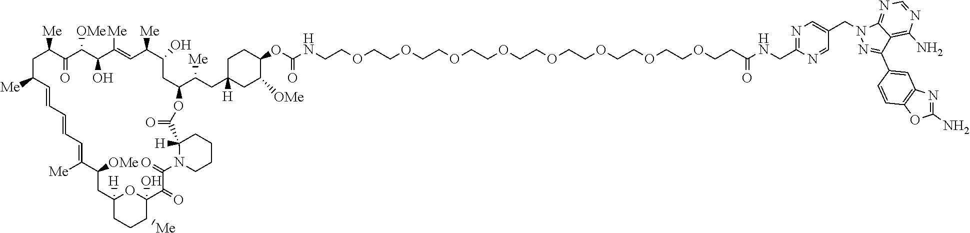 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ncc(Cn4nc(-c5ccc6oc(N)nc6c5)c5c(N)ncnc54)cn3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 139 | C₈₉H₁₃₂N₁₂O₂₄ | [M + H] = 1753.96 | [M + H] = 1753.7 |
| 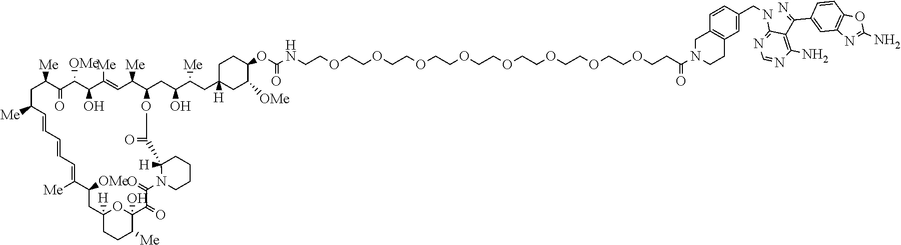 [SMILES: CO[C@H]1C[C@@H]2CC[C@@H](C)[C@@](O)(O2)C(=O)C(=O)N2CCCC[C@H]2C(=O)O[C@H](C[C@H](O)[C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)[C@H](C)/C=C(\C)[C@@H](O)[C@@H](OC)C(=O)[C@H](C)C[C@H](C)/C=C/C=C/C=C/1C] Example 140 | C₉₃H₁₃₆N₁₀O₂₄ | [M + H] = 1777.98 | [M + H] = 1777.7 |
| 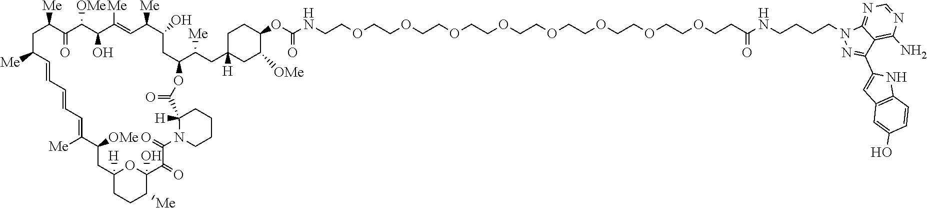 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4cc5cc(O)ccc5[nH]4)c4c(N)ncnc43)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 141 | C₈₈H₁₃₅N₉O₂₄ | [M + H] = 1702.97 | [M + H] = 1702.9 |
| 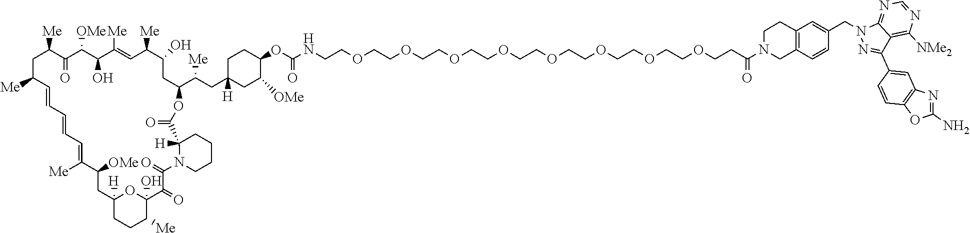 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N(C)C)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 142 | C₉₅H₁₄₀N₁₀O₂₄ | [M + H] = 1806.01 | [M + H] = 1805.8 |
| 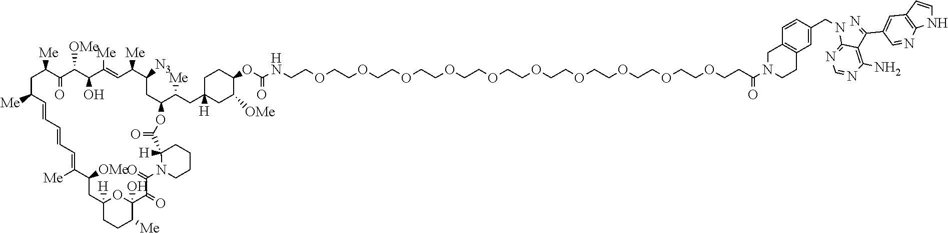 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] Example 143 | C₉₇H₁₄₃N₁₃O₂₄ | [M + H] = 1875.04 | [M + H] = 1874.9 |
| 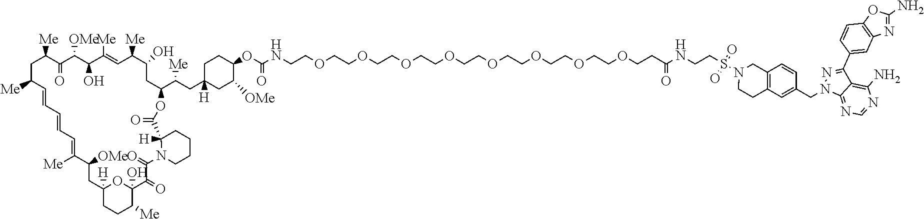 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 144 | C₉₅H₁₄₁N₁₁O₂₆S | [M + H] = 1884.98 | [M + H] = 1884.9 |
| 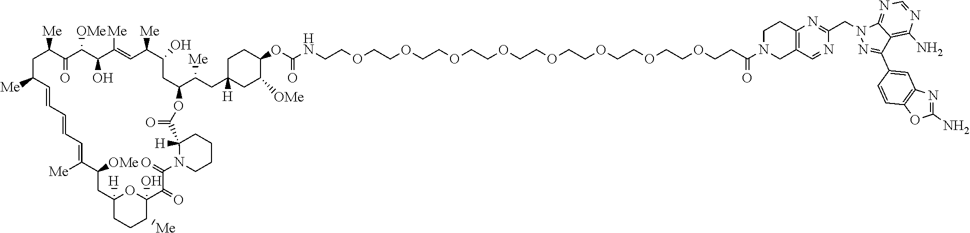 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4nc(Cn5nc(-c6ccc7oc(N)nc7c6)c6c(N)ncnc65)ncc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 145 | C₉₁H₁₃₄N₁₂O₂₄ | [M + H] = 1779.97 | [M + H] = 1779.8 |
| 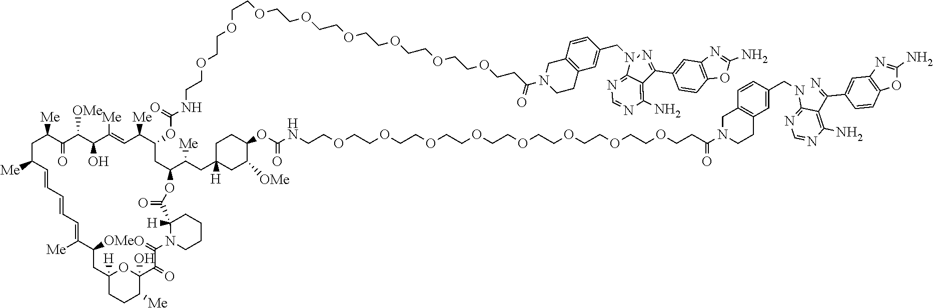 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@H](C)/C=C(\C)[C@@H](O)[C@@H](OC)C(=O)[C@H](C)C[C@H](C)/C=C/C=C/C=C/1C] Example 146 | C₁₃₅H₁₉₁N₁₉O₃₅ | [M + 2H]/2 = 1320.19 | [M+ 2H]/2 = 1320.8 |
| 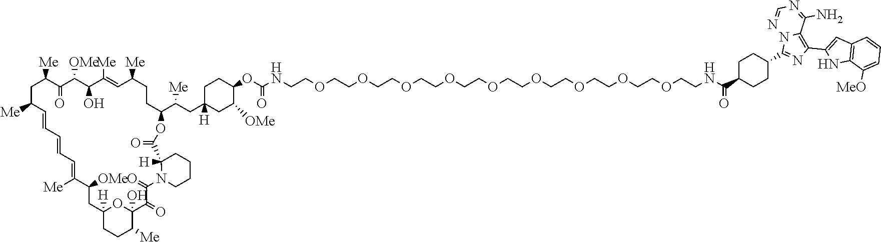 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6CC[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] Example 147 | C₉₃H₁₄₃N₉O₂₄ | [M + H] = 1771.03 | [M + H] = 1770.8 |
| 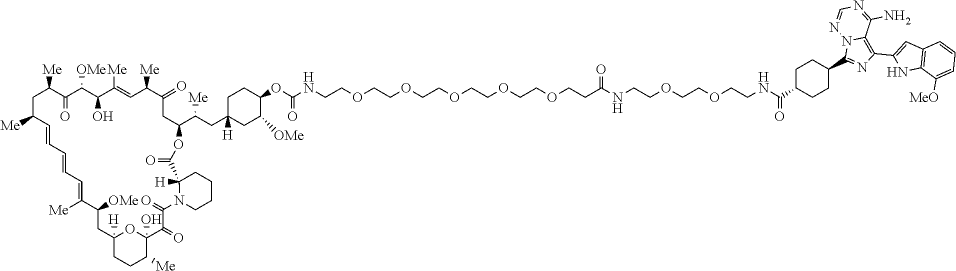 [SMILES: COc1cccc2cc(-c3nc([C@H]4CC[C@H](C(=O)NCCOCCOCCNC(=O)CCOCCOCCOCCOCCOCCNC(=O)O[C@@H]5CC[C@@H](C[C@@H](C)[C@@H]6CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] Example 148 | C₉₂H₁₃₈N₁₀O₂₄ | [M + H] = 1768.00 | [M + H] = 1767.8 |

### General Procedure 3: Coupling of a Halide Containing PEG Ester and an Amine Containing Pre-Linker Followed by Ester Deprotection

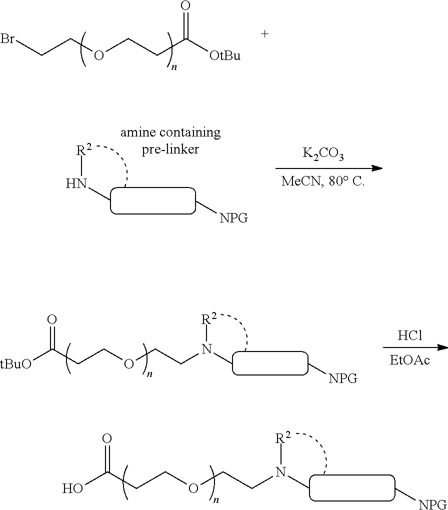

### Step 1

To a 0.1 M solution of amine containing pre-linker (1.0 equiv) in MeCN was added K₂CO₃ (2.0 equiv) followed by halide containing PEG ester (1.0 equiv). The reaction was stirred at 80° C. until consumption of amine containing pre-linker, as indicated by LCMS analysis. The reaction was then purified by silica gel chromatography to afford the product.

### Step 2

To a 0.1 M solution of PEG tert-butyl ester (1.0 equiv) in EtOAc was added a solution of HCl in EtOAc. The resulting suspension was stirred at room temperature until consumption of the PEG ester, as indicated by LCMS analysis. The reaction was then concentrated under reduced pressure to afford the product.

### Intermediate B1-1. 1-(4-(5-((1,3-dioxoisoindolin-2-yl)methyl)pyrimidin-2-yl)piperazin-1-yl)-3,6,9,12-tetraoxapentadecan-15-oic acid

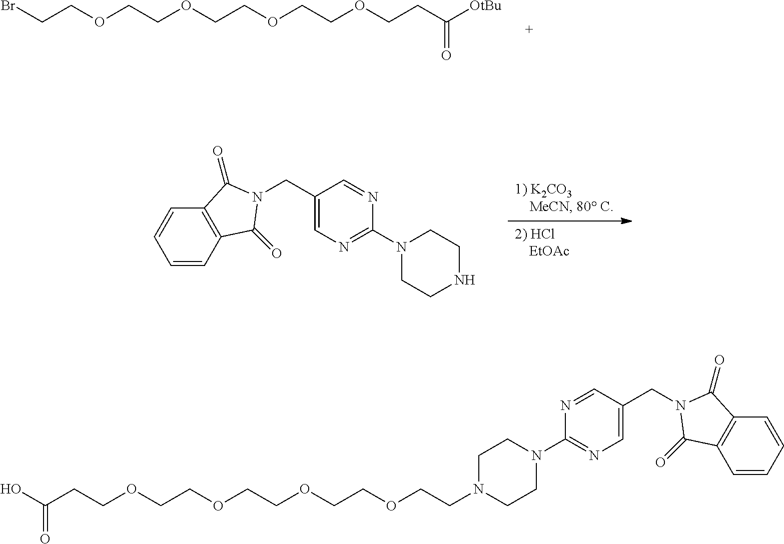

### Step 1: Synthesis of tert-butyl 1-(4-(5-((1,3-dioxoisoindolin-2-yl)methyl)pyrimidin-2-yl)piperazin-1-yl)-3,6,9,12-tetraoxapentadecan-15-oate

To a mixture of 2-((2-(piperazin-1-yl)pyrimidin-5-yl)methyl)isoindoline-1,3-dione (7.97 g, 24.66 mmol, 1.0 equiv) in MeCN (200 mL) was added K₂CO₃ (6.82 g, 49.31 mmol, 2.0 equiv) followed by tert-butyl 1-bromo-3,6,9,12-tetraoxapentadecan-15-oate (9.5 g, 24.66 mmol, 1.0 equiv). The reaction mixture was heated to 85° C. and stirred for 15 h. The mixture was then cooled to room temperature and filtered. The filtrate was concentrated under reduced pressure and the residue was purified by silica gel chromatography (0→20% EtOAc/MeOH) to give the product (11.5 g, 74.3% yield) a light yellow liquid.

### Step 2: Synthesis of 1-(4-(5-((1,3-dioxoisoindolin-2-yl)methyl)pyrimidin-2-yl)piperazin-1-yl)-3,6,9,12-tetraoxapentadecan-15-oic acid

To a solution of tert-butyl 1-(4-(5-((1,3-dioxoisoindolin-2-yl)methyl)pyrimidin-2-yl)piperazin-1-yl)-3,6,9,12-tetraoxapentadecan-15-oate (3.5 g, 5.58 mmol, 1.0 equiv) in EtOAc (50 mL) was added a solution of HCl in EtOAc (500 mL). The mixture was stirred at room temperature for 3 h. The mixture was then concentrated under reduced pressure to give the product (5.3 g, 78.2% yield. HCl) as a white solid. LCMS (ESI) m/z: [M+H] calcd for C₂₈H₃₇N₅O₈: 572.27; found 572.4.

Following General Procedure 3, but using the appropriate halide containing PEG and amine containing pre-linkers in Table 4, the Intermediates B1 in Table 8 were prepared:

TABLE 8
Additional protected amines prepared

| | Molecular | Calculated | Observed |
|---|---|---|---|
| Structure | Formula | MW | MW |
| 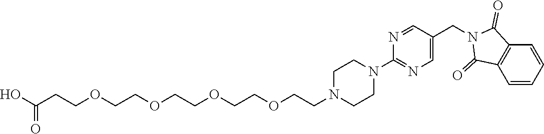 [SMILES: O=C(O)CCOCCOCCOCCOCCN1CCN(c2ncc(CN3C(=O)c4ccccc4C3=O)cn2)CC1] | C₂₈H₃₇N₅O₈ | [M + H] = 572.27 | [M + H] = 572.4 |
| Intermediate B1-1 | | | |

### General Procedure 4: Coupling of a PEG Carboxylic Acid and an Amine Containing Active Site Inhibitor Followed by Amine Deprotection

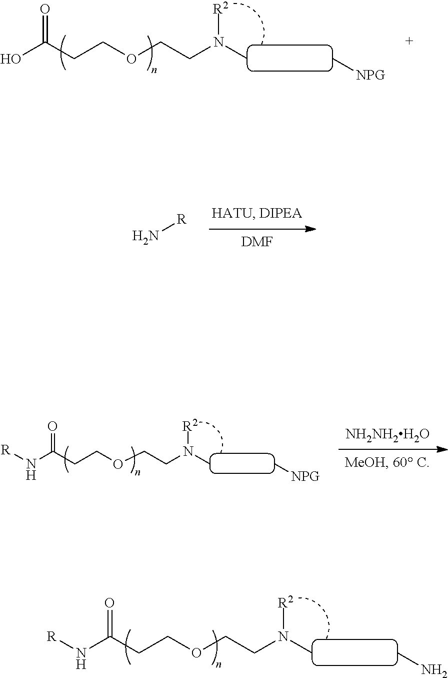

### Step 1

To a 0.15 M solution of PEG carboxylic acid (1.0 equiv) in DMF was added HATU (1.3 equiv) and DIPEA (5.0 equiv). After stirring for 30 min, the amine containing active site inhibitor (1.2 equiv) was added. The reaction was stirred at room temperature until consumption of PEG carboxylic acid, as indicated by LCMS. The reaction was then purified by reverse phase chromatography to afford the product.

### Step 2

To a 0.1 M solution of phthalimide protected amine (1.0 equiv) in MeOH at 0° C. was added NH₂NH₂·H₂O (4.0 equiv). The resulting mixture was stirred at 60° C. until consumption of the phthalimide protected amine, as indicated by LCMS analysis. The reaction was then purified by reverse phase chromatography to afford the product.

### Intermediate B2-1. N-(4-(4-amino-3-(2-aminobenzo[d]oxazol-5-yl)-1H-pyrazolo[3,4-d]pyrimidin-1-yl)butyl)-1-(4-(5-(aminomethyl)pyrimidin-2-yl)piperazin-1-yl)-3,6,9,12-tetraoxapentadecan-15-amide

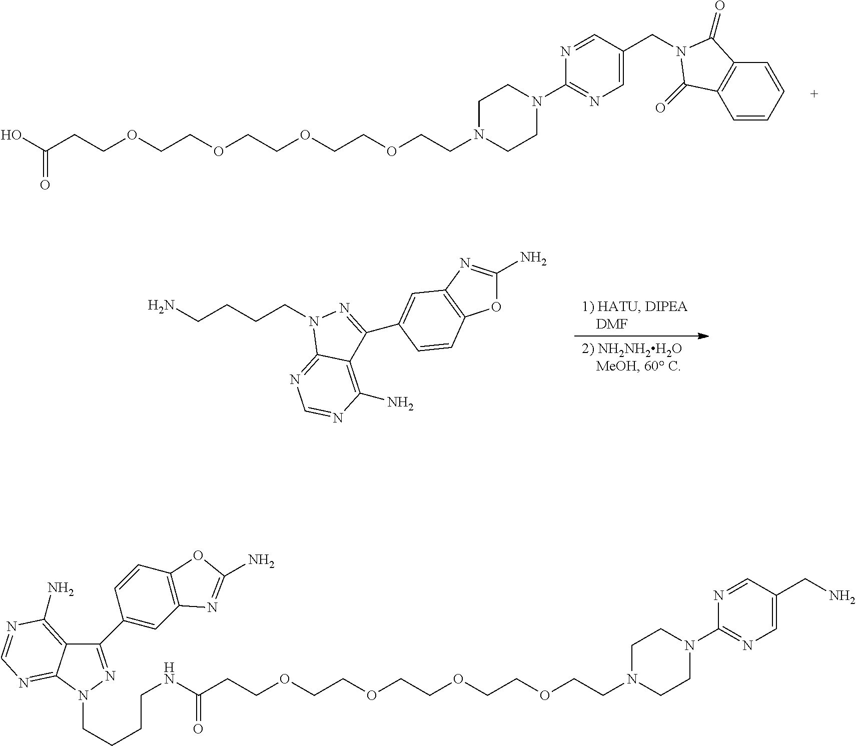

### Step 1: Synthesis of N-(4-(4-amino-3-(2-aminobenzo[d]oxazol-5-yl)-1H-pyrazolo[3,4-d]pyrimidin-1-yl)butyl)-1-(4-(5-((1,3-dioxoisoindolin-2-yl)methyl)pyrimidin-2-yl)piperazin-1-yl)-3,6,9,12-tetraoxapentadecan-15-amide

To a mixture of 1-(4-(5-((1,3-dioxoisoindolin-2-yl)methyl)pyrimidin-2-yl)piperazin-1-yl)-3,6,9,12-tetraoxapentadecan-15-oic acid (3 g, 4.93 mmol, 1.0 equiv, HCl) in DMF (30 mL) was added HATU (12.11 μL, 6.41 mmol, 1.3 equiv) and DIPEA (4.30 mL, 24.67 mmol, 5.0 equiv). After 30 min, 5-(4-amino-1-(4-aminobutyl)-1H-pyrazolo[3,4-d]pyrimidin-3-yl)benzo[d]oxazol-2-amine (4.03 g, 5.92 mmol, 1.2 equiv, 3TFA) was added. The mixture was stirred at room temperature for 3 h. The reaction mixture was then purified by prep-HPLC (MeCN/H₂O) to give the product (5.4 g, 81.2% yield, 4TFA) as a light red solid. LCMS (ESI) m/z: [M+2H]/2 calcd for C₄₄H₅₃N₁₃O₈: 446.71; found 447.0.

### Step 2: Synthesis of N-(4-(4-amino-3-(2-aminobenzo[d]oxazol-5-yl)-1H-pyrazolo[3,4-d]pyrimidin-1-yl)butyl)-1-(4-(5-(aminomethyl)pyrimidin-2-yl)piperazin-1-yl)-3,6,9,12-tetraoxapentadecan-15-amide

To a mixture of N-(4-(4-amino-3-(2-aminobenzo[d]oxazol-5-yl)-1H-pyrazolo[3,4-d]pyrimidin-1-yl)butyl)-1-(4-(5-((1,3-dioxoisoindolin-2-yl)methyl)pyrimidin-2-yl)piperazin-1-yl)-3,6,9,12-tetraoxapentadecan-15-amide (4 g, 2.97 mmol, 1.0 equiv, 4TFA) in MeOH (25 mL) at 0° C. was added NH₂NH₂·H₂O (588.63 μL, 11.87 mmol, 4.0 equiv). The mixture was stirred at 60° C. for 2 h. The mixture was then cooled to room temperature and filtered, and the filter cake was washed with MeOH (5 mL). The filtrate was concentrated under reduced pressure and the residue was purified by prep-H PLC (MeCN/H₂O) to give the product (700 mg, 24.5% yield, TFA) as a white solid. LCMS (ESI) m/z: [M+2H]/2 calcd for C₃₆H₅₁N₁₃O₆: 381.71; found 381.8.

Following General Procedure 4, but using the appropriate Intermediate B1 in Table 8 and amine containing active site inhibitors in Table 2, the Intermediates B2 in Table 9 were prepared:

TABLE 9
Additional amines prepared

| | | Calcu- | Ob- |
|---|---|---|---|
| | Molecular | lated | served |
| Structure | Formula | MW | MW |
| 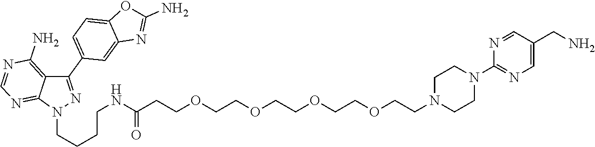 [SMILES: NCc1cnc(N2CCN(CCOCCOCCOCCOCCC(=O)NCCCCn3nc(-c4ccc5oc(N)nc5c4)c4c(N)ncnc43)CC2)nc1] | C₃₆H₅₁N₁₃O₆ | [M + 2H]/2 = 381.71 | [M + 2H]/2 = 381.8 |
| Intermediate B2-1 | | | |
| 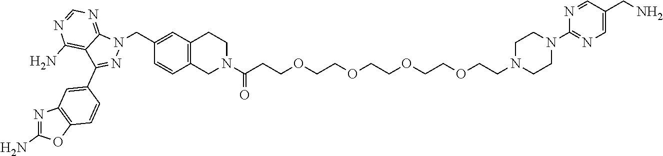 [SMILES: NCc1cnc(N2CCN(CCOCCOCCOCCOCCC(=O)N3CCc4cc(Cn5nc(-c6ccc7oc(N)nc7c6)c6c(N)ncnc65)ccc4C3)CC2)nc1] | C₄₂H₅₃N₁₃O₆ | [M + H] = 836.43 | [M + H] = 836.4 |
| Intermediate B2-2 | | | |
| 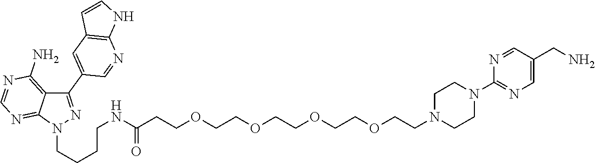 [SMILES: NCc1cnc(N2CCN(CCOCCOCCOCCOCCC(=O)NCCCCn3nc(-c4cnc5[nH]ccc5c4)c4c(N)ncnc43)CC2)nc1] | C₃₆H₅₁N₁₃O₅ | [M + 2H]/2 = 373.72 | [M + 2H]/2 = 737.7 |
| Intermediate B2-3 | | | |

### General Procedure 5: Coupling of a Halide Containing PEG Carboxylic Acid and an Amine Containing Active Site Inhibitor.

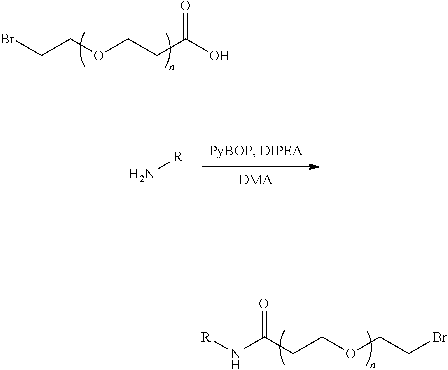

To a 0.1 M solution of amine containing active site inhibitor (1.0 equiv) and PEG containing carboxylic acid (1.2 equiv) in DMA was added DIPEA (4.0 equiv) followed by PyBOP (1.3 equiv). The reaction was stirred until consumption of amine containing active site inhibitor, as indicated by LCMS. The reaction was then purified by reverse phase HPLC to afford the product.

### Intermediate B3-1. 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-bromo-3,6,9,12,15-pentaoxaoctadecan-18-one

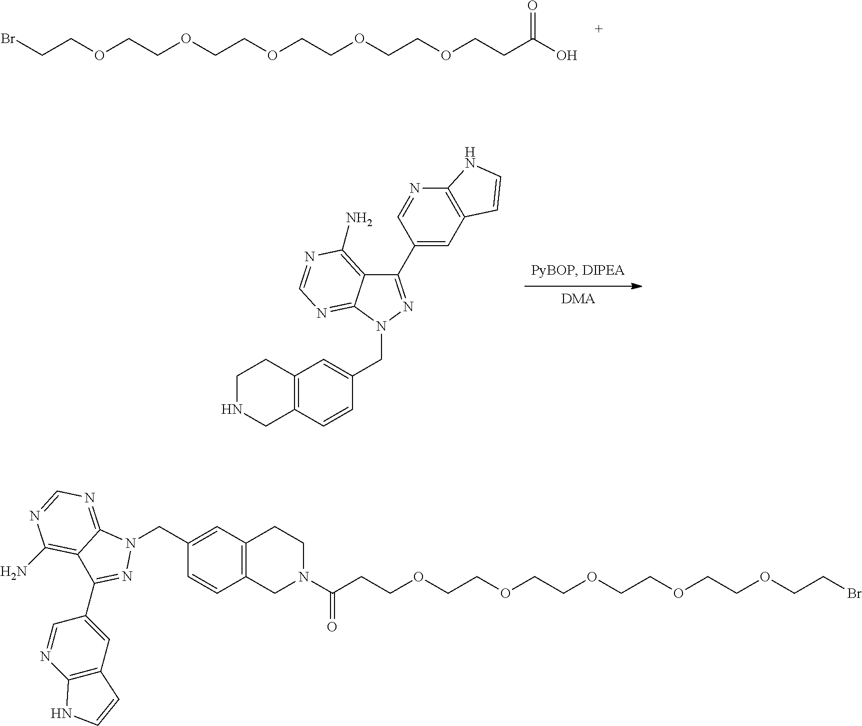

To a solution of 1-bromo-3,6,9,12,15-pentaoxaoctadecan-18-oic acid (105 mg. 282 μmol, 1.2 equiv) and 3-{1H-pyrrolo[2,3-b]pyridin-5-yl}-1-[(1,2,3,4-tetrahydroisoquinolin-6-yl)methyl]-1H-pyrazolo[3,4-d]pyrimidin-4-amine (120 mg, 235 μmol, 1.0 equiv) in DMA (2.34 mL) was added DIPEA (163 μL, 940 μmol, 4.0 equiv) followed by PyBOP (158 mg, 305 μmol, 1.3 equiv). The resulting solution was stirred at room temperature for 3 h then purified by reverse phase HPLC (10→98% MeCN+0.1% formic acid/H₂O+0.1% formic acid) to afford the product (82.7 mg, 47% yield). LCMS (ESI) m/z: [M+H] calcd for C₃₅H₄₃BrNO₆: 751.26; found 751.2.

Following General Procedure 5, but using the appropriate halide containing PEG carboxylic acid and amine containing active site inhibitors in Table 2, the Intermediates B3 in Table 10 were prepared:

TABLE 10
Additional PEG halides prepared

| | Molecular | Calculated | Observed |
|---|---|---|---|
| Structure | Formula | MW | MW |
| 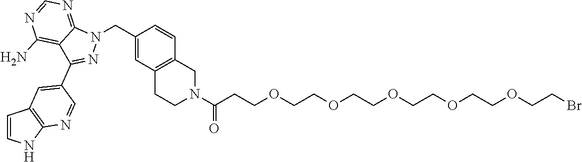 [SMILES: Nc1ncnc2c1c(-c1cnc3[nH]ccc3c1)nn2Cc1ccc2c(c1)CCN(C(=O)CCOCCOCCOCCOCCOCCBr)C2] | C₃₅H₄₃BrN₈O₆ | [M + H] = 751.26 | [M + H] = 751.2 |
| Intermediate B3-1 | | | |
| 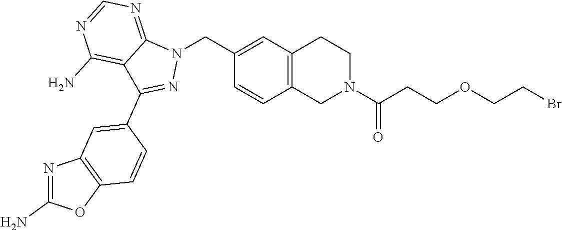 [SMILES: Nc1nc2cc(-c3nn(Cc4ccc5c(c4)CCN(C(=O)CCOCCBr)C5)c4ncnc(N)c34)ccc2o1] | C₂₇H₂₇BrN₈O₃ | [M + H] = 591.15 | [M + H] = 591.2 |
| Intermediate B3-2 | | | |

General Procedure 6: Displacement of a PEG Halide with an Amine Containing Post Linker and Deprotection of the Amine

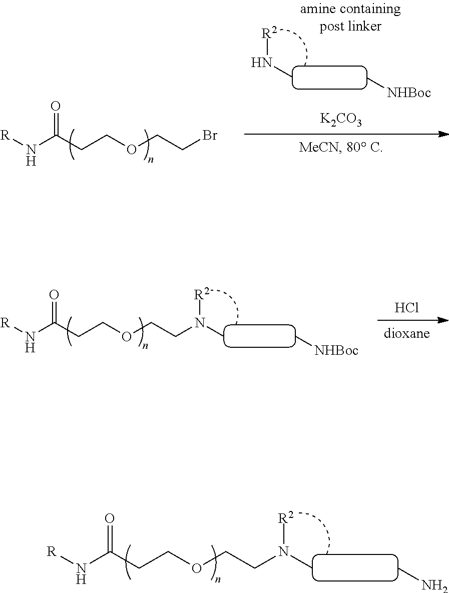

### Step 1

To a 0.1 M solution of halide containing PEG (1.0 equiv) in MeCN was added K₂CO₃ (3.0 equiv) followed by amine containing post linker (1.2 equiv). The resulting suspension was heated to 80° C. and stirred until consumption of the PEG halide, as indicated by LCMS analysis. The reaction was cooled to room temperature and then purified by silica gel chromatography to afford the product.

### Step 2

To a 0.07 M solution of N-Boc protected amine (1.0 equiv) in dioxane was added HCl (4 M in dioxane, 10.0 equiv). The reaction was stirred until consumption of N-Boc protected amine, as indicated by LCMS analysis. The reaction was then concentrated under reduced pressure to afford the product.

### Intermediate B2-4. 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-(4-{5H,6H,7H,8H-pyrido[4,3-d]pyrimidin-2-yl}piperazin-1-yl)-3,6,9,12,15-pentaoxaoctadecan-18-one

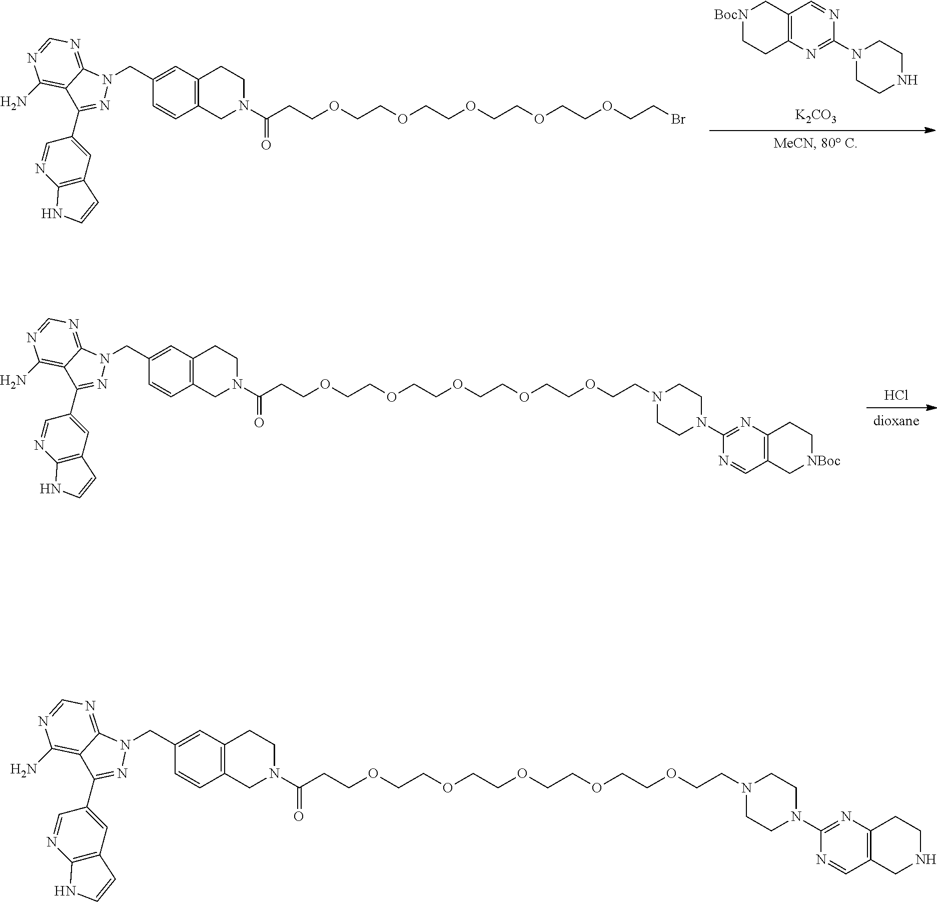

### Step 1: Synthesis of tert-butyl 2-[4-(18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-18-oxo-3,6,9,12,15-pentaoxaoctadecan-1-yl)piperazin-1-yl]-5H,6H,7H,8H-pyrido[4,3-d]pyrimidine-6-carboxylate

To a suspension of 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-bromo-3,6,9,12,15-pentaoxaoctadecan-18-one (82.7 mg, 110 μmol, 1.0 equiv) in MeCN (1.09 mL) was added K₂CO₃ (45.6 mg, 330 μmol, 3.0 equiv) followed by tert-butyl 2-(piperazin-1-yl)-5H,6H,7H,8H-pyrido[4,3-d]pyrimidine-6-carboxylate (42.1 mg, 132 μmol, 1.2 equiv). The resulting suspension was heated to 80° C. for 8 h, then purified by silica gel chromatography (0→20% MeOH/DCM) to afford the product (75.1 mg, 70% yield). LCMS (ESI) m/z: [M+H] calcd for C₉₁H₆₇N₁₃O₈: 990.53; found 990.5.

### Step 2: Synthesis of 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-(4-{5H,6H,7H,8H-pyrido[4,3-d]pyrimidin-2-yl}piperazin-1-yl)-3,6,9,12,15-pentaoxaoctadecan-18-one

To a solution of tert-butyl 2-[4-(18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-18-oxo-3,6,9,12,15-pentaoxaoctadecan-1-yl)piperazin-1-yl]-5H,6H,7H,8H-pyrido[4,3-d]pyrimidine-6-carboxylate (75.1 mg, 75.8 μmol, 1.0 equiv) in dioxane (1 mL) was added HCl (4 M in dioxane, 472 μL, 1.89 mmol, 10.0 equiv). The solution was stirred at room temperature for 45 min, then concentrated under reduced pressure to afford the product. LCMS (ESI) m/z: [M+Na] calcd for C₄₆H₅₉N₁₃O₆: 912.46; found 912.5.

Following General Procedure 6, but using the appropriate PEG carboxylic acid and amine containing active site inhibitors in Table 2, the Intermediates B2 in Table 11 were prepared:

TABLE 11
Additional amines prepared

| | Molecular | Calculated | Observed |
|---|---|---|---|
| Structure | Formula | MW | MW |
| 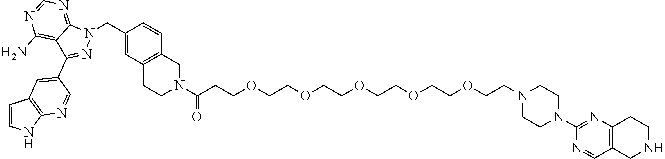 [SMILES: Nc1ncnc2c1c(-c1cnc3[nH]ccc3c1)nn2Cc1ccc2c(c1)CCN(C(=O)CCOCCOCCOCCOCCOCCN1CCN(c3ncc4c(n3)CCNC4)CC1)C2] | C₄₆H₅₉N₁₃O₆ | [M + H] = 890.48 | [M + H] = 890.5 |
| Intermediate B2-4 | | | |

Following General Procedure 1, but using the appropriate carboxylic acid PEG tert-butyl ester and amine containing active site inhibitors in Table 2, the Intermediates B4 in Table 12 were prepared:

TABLE 12
Additional amines prepared

| | Molecular | Calculated | Observed |
|---|---|---|---|
| Structure | Formula | MW | MW |
| 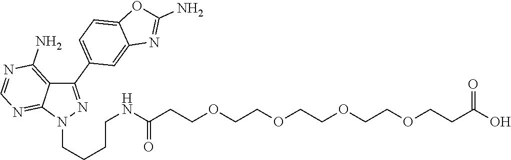 [SMILES: Nc1nc2cc(-c3nn(CCCCNC(=O)CCOCCOCCOCCOCCC(=O)O)c4ncnc(N)c34)ccc2o1] | C₂₈H₃₈N₈O₈ | [M + H] = 615.29 | [M + H] = 615.1 |
| Intermediate B4-1 | | | |

Following General Procedure 1, but using the appropriate Intermediates B4 in Table 12 and amine containing pre-linkers in Table 4, the Intermediates B2 in Table 13 were prepared:

TABLE 13
Additional amines prepared

| | Molecular | Calculated | Observed |
|---|---|---|---|
| Structure | Formula | MW | MW |
| 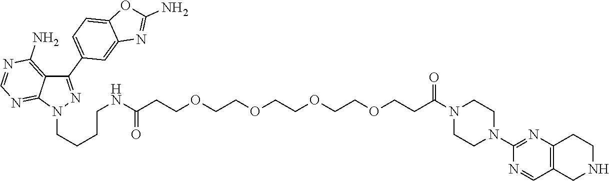 [SMILES: Nc1nc2cc(-c3nn(CCCCNC(=O)CCOCCOCCOCCOCCC(=O)N4CCN(c5ncc6c(n5)CCNC6)CC4)c4ncnc(N)c34)ccc2o1] | C₃₉H₅₃N₁₃O₇ | [M + H] = 816.43 | [M + H] = 816.4 |
| Intermediate B2-5 | | | |

Following General Procedure 1, but using the appropriate Intermediates A1 and amine containing pre-linkers in Table 4, the Intermediates B32 in Table 14 were prepared:

TABLE 14
Additional amines prepared

| | Molecular | Calculated | Observed |
|---|---|---|---|
| Structure | Formula | MW | MW |
| 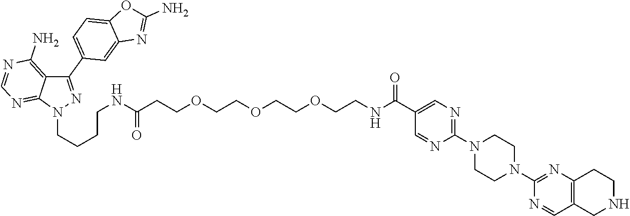 [SMILES: Nc1nc2cc(-c3nn(CCCCNC(=O)CCOCCOCCOCCNC(=O)c4cnc(N5CCN(c6ncc7c(n6)CCNC7)CC5)nc4)c4ncnc(N)c34)ccc2o1] | C₄₁H₅₂N₁₆O₆ | [M + H] = 865.44 | [M + H] = 865.2 |
| Intermediate B2-6 | | | |
| 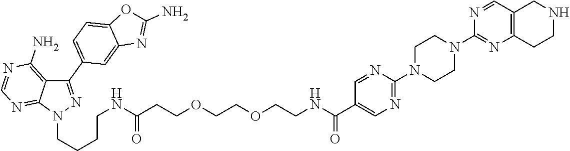 [SMILES: Nc1nc2cc(-c3nn(CCCCNC(=O)CCOCCOCCNC(=O)c4cnc(N5CCN(c6ncc7c(n6)CCNC7)CC5)nc4)c4ncnc(N)c34)ccc2o1] | C₃₉H₄₈N₁₆O₅ | [M + H] = 821.41 | [M + H] = 821.2 |
| Intermediate B2-7 | | | |
| 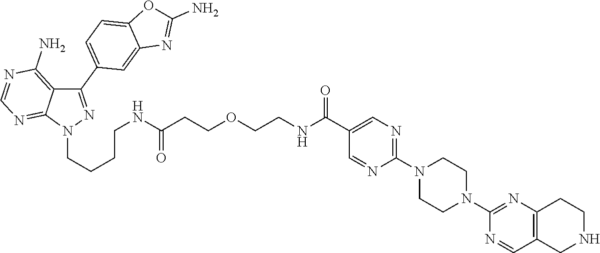 [SMILES: Nc1nc2cc(-c3nn(CCCCNC(=O)CCOCCNC(=O)c4cnc(N5CCN(c6ncc7c(n6)CCNC7)CC5)nc4)c4ncnc(N)c34)ccc2o1] | C₃₇H₄₄N₁₆O₄ | [M + H] = 777.38 | [M + H] = 777.3 |
| Intermediate B2-8 | | | |
| 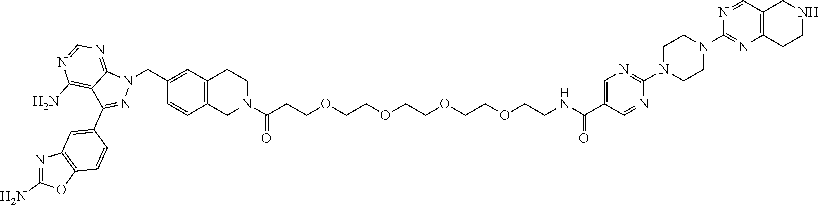 [SMILES: Nc1nc2cc(-c3nn(Cc4ccc5c(c4)CCN(C(=O)CCOCCOCCOCCOCCNC(=O)c4cnc(N6CCN(c7ncc8c(n7)CCNC8)CC6)nc4)C5)c4ncnc(N)c34)ccc2o1] | C₄₉H₅₈N₁₆O₇ | [M + H] = 983.48 | [M + H] = 983.4 |
| Intermediate B2-9 | | | |
| 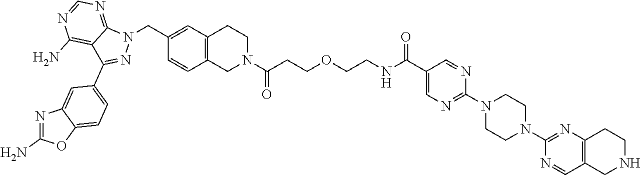 [SMILES: Nc1nc2cc(-c3nn(Cc4ccc5c(c4)CCN(C(=O)CCOCCNC(=O)c4cnc(N6CCN(c7ncc8c(n7)CCNC8)CC6)nc4)C5)c4ncnc(N)c34)ccc2o1] | C₄₃H₄₆N₁₆O₄ | [M + H] = 851.40 | [M + H] = 851.4 |
| Intermediate B2-10 | | | |
| 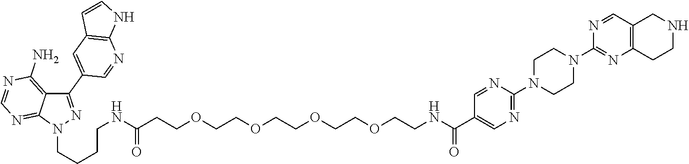 [SMILES: Nc1ncnc2c1c(-c1cnc3[nH]ccc3c1)nn2CCCCNC(=O)CCOCCOCCOCCOCCNC(=O)c1cnc(N2CCN(c3ncc4c(n3)CCNC4)CC2)nc1] | C₄₃H₅₆N₁₆O₆ | [M + H] = 893.47 | [M + H] = 893.3 |
| Intermediate B2-11 | | | |
| 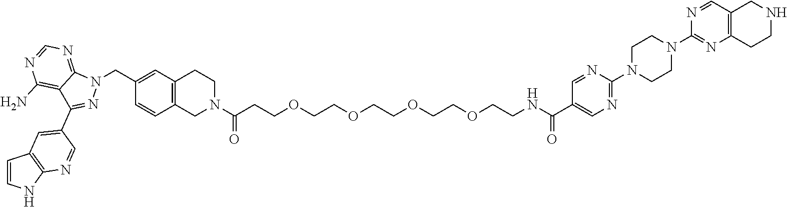 [SMILES: Nc1ncnc2c1c(-c1cnc3[nH]ccc3c1)nn2Cc1ccc2c(c1)CCN(C(=O)CCOCCOCCOCCOCCNC(=O)c1cnc(N3CCN(c4ncc5c(n4)CCNC5)CC3)nc1)C2] | C₄₉H₅₈N₁₆O₆ | [M + Na] = 989.46 | [M + Na] = 989.4 |
| Intermediate B2-12 | | | |
| 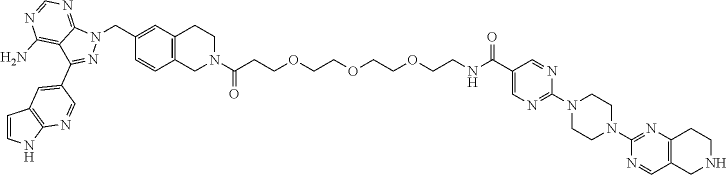 [SMILES: Nc1ncnc2c1c(-c1cnc3[nH]ccc3c1)nn2Cc1ccc2c(c1)CCN(C(=O)CCOCCOCCOCCNC(=O)c1cnc(N3CCN(c4ncc5c(n4)CCNC5)CC3)nc1)C2] | C₄₇H₅₄N₁₆O₅ | [M + H] = 923.46 | [M + H] = 923.4 |
| Intermediate B2-13 | | | |
| 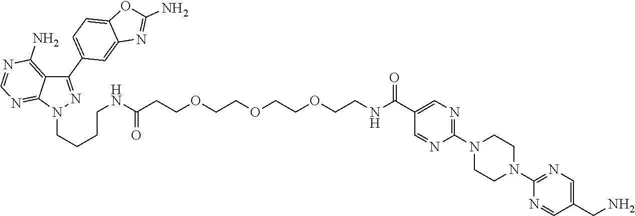 [SMILES: NCc1cnc(N2CCN(c3ncc(C(=O)NCCOCCOCCOCCC(=O)NCCCCn4nc(-c5ccc6oc(N)nc6c5)c5c(N)ncnc54)cn3)CC2)nc1] | C₃₉H₅₀N₁₆O₆ | [M + H] = 839.42 | [M + H] = 839.3 |
| Intermediate B2-14 | | | |
| 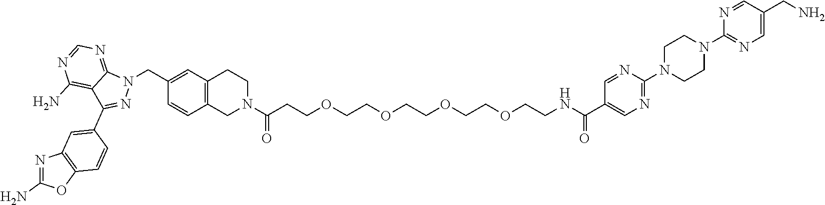 [SMILES: NCc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)cn3)CC2)nc1] | C₄₇H₅₆N₁₆O₇ | [M + H] = 957.46 | [M + H] = 957.7 |
| Intermediate B2-15 | | | |
| 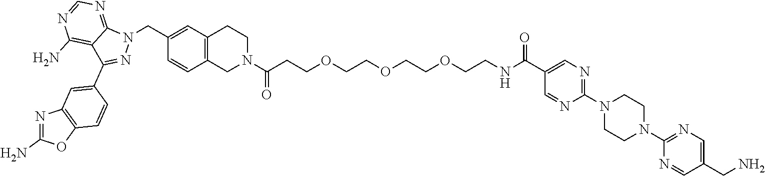 [SMILES: NCc1cnc(N2CCN(c3ncc(C(=O)NCCOCCOCCOCCC(=O)N4CCc5cc(Cn6nc(-c7ccc8oc(N)nc8c7)c7c(N)ncnc76)ccc5C4)cn3)CC2)nc1] | C₄₅H₅₂N₁₆O₆ | [M + Na] = 935.42 | [M + Na] = 935.3 |
| Intermediate B2-16 | | | |
| 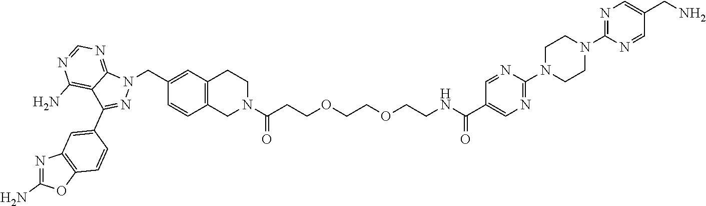 [SMILES: NCc1cnc(N2CCN(c3ncc(C(=O)NCCOCCOCCC(=O)N4CCc5cc(Cn6nc(-c7ccc8oc(N)nc8c7)c7c(N)ncnc76)ccc5C4)cn3)CC2)nc1] | C₄₃H₄₈N₁₆O₅ | [M + Na] = 891.39 | [M + Na] = 891.4 |
| Intermediate B2-17 | | | |
| 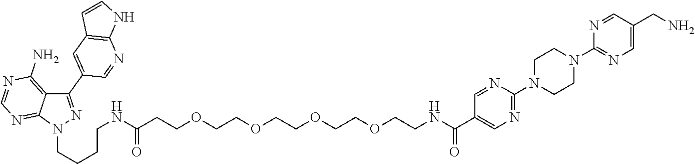 [SMILES: NCc1cnc(N2CCN(c3ncc(C(=O)NCCOCCOCCOCCOCCC(=O)NCCCCn4nc(-c5cnc6[nH]ccc6c5)c5c(N)ncnc54)cn3)CC2)nc1] | C₄₁H₅₄N₁₆O₆ | [M + H] = 867.45 | [M + H] = 867.3 |
| Intermediate B2-18 | | | |
| 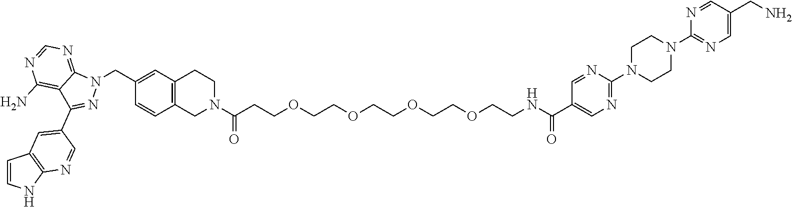 [SMILES: NCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)cn3)CC2)nc1] | C₄₇H₅₆N₁₆O₆ | [M + H] = 941.47 | [M + H] = 941.2 |
| Intermediate B2-19 | | | |
| 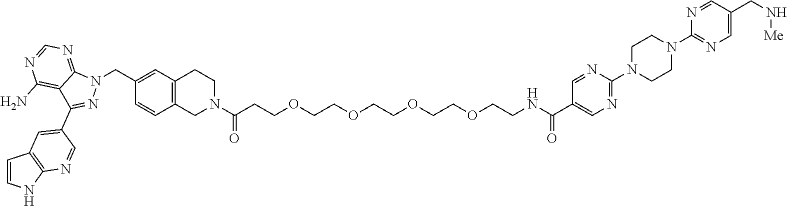 [SMILES: CNCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)cn3)CC2)nc1] | C₄₈H₅₈N₁₆O₆ | [M + H] = 955.48 | [M + H] = 955.2 |
| Intermediate B2-20 | | | |
| 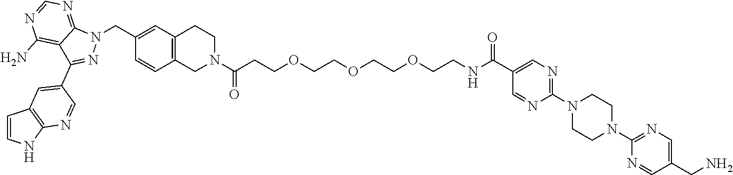 [SMILES: NCc1cnc(N2CCN(c3ncc(C(=O)NCCOCCOCCOCCC(=O)N4CCc5cc(Cn6nc(-c7cnc8[nH]ccc8c7)c7c(N)ncnc76)ccc5C4)cn3)CC2)nc1] | C₄₅H₅₂N₁₆O₅ | [M + H] = 897.44 | [M + H] = 897.3 |
| Intermediate B2-21 | | | |
| 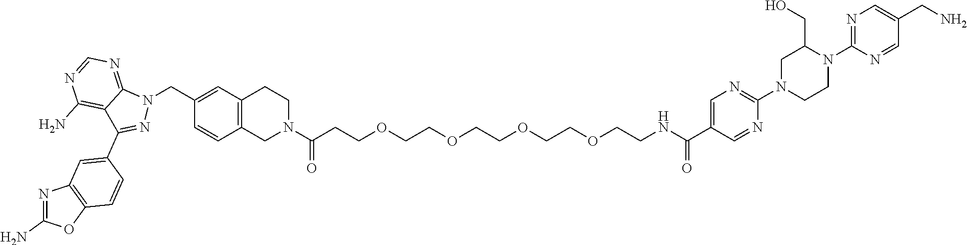 [SMILES: NCc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)cn3)CC2CO)nc1] | C₄₈H₅₈N₁₆O₈ | [M + H] = 987.47 | [M + H] = 987.42 |
| Intermediate B2-22 | | | |
| 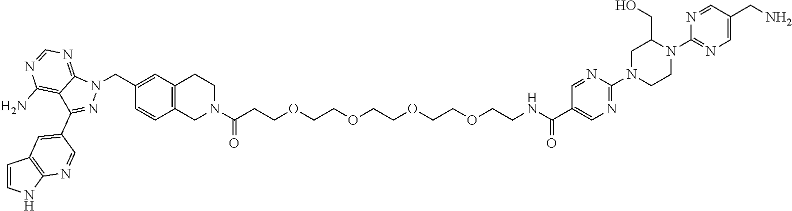 [SMILES: NCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)cn3)CC2CO)nc1] | C₄₈H₅₈N₁₆O₇ | [M + H] = 971.48 | [M + H] = 971.31 |
| Intermediate B2-23 | | | |
| 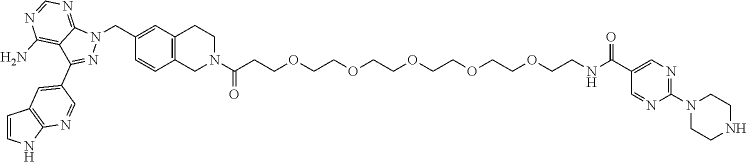 [SMILES: Nc1ncnc2c1c(-c1cnc3[nH]ccc3c1)nn2Cc1ccc2c(c1)CCN(C(=O)CCOCCOCCOCCOCCOCCNC(=O)c1cnc(N3CCNCC3)nc1)C2] | C₄₄H₅₅N₁₃O₇ | [M + H] = 878.44 | [M + H] = 878.5 |
| Intermediate B2-24 | | | |
| 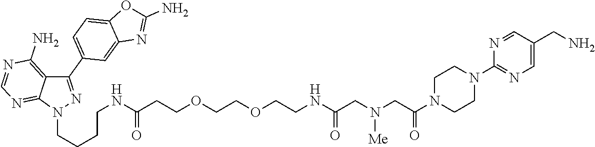 [SMILES: CN(CC(=O)NCCOCCOCCC(=O)NCCCCn1nc(-c2ccc3oc(N)nc3c2)c2c(N)ncnc21)CC(=O)N1CCN(c2ncc(CN)cn2)CC1] | C₃₇H₅₁N₁₅O₆ | [M + H] = 802.42 | [M + H] = 802.4 |
| Intermediate B2-25 | | | |
| 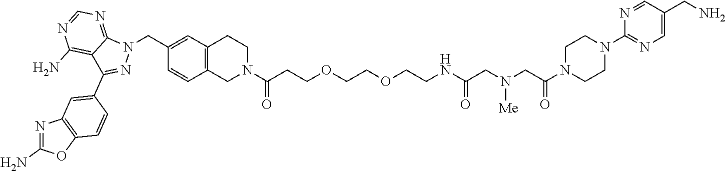 [SMILES: CN(CC(=O)NCCOCCOCCC(=O)N1CCc2cc(Cn3nc(-c4ccc5oc(N)nc5c4)c4c(N)ncnc43)ccc2C1)CC(=O)N1CCN(c2ncc(CN)cn2)CC1] | C₄₃H₅₃N₁₅O₆ | [M + H] = 876.44 | [M + H] = 876.4 |
| Intermediate B2-26 | | | |
| 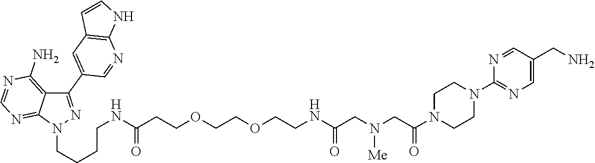 [SMILES: CN(CC(=O)NCCOCCOCCC(=O)NCCCCn1nc(-c2cnc3[nH]ccc3c2)c2c(N)ncnc21)CC(=O)N1CCN(c2ncc(CN)cn2)CC1] | C₃₇H₅₁N₁₅O₅ | [M + H] = 786.43 | [M + H] = 786.5 |
| Intermediate B2-27 | | | |
| 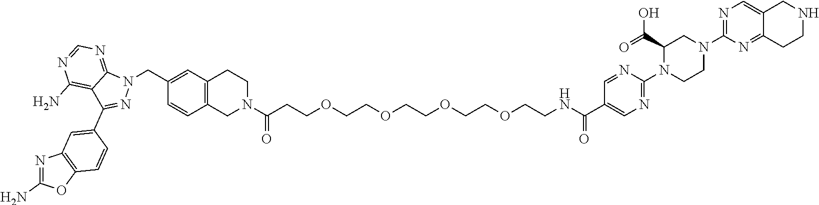 [SMILES: Nc1nc2cc(-c3nn(Cc4ccc5c(c4)CCN(C(=O)CCOCCOCCOCCOCCNC(=O)c4cnc(N6CCN(c7ncc8c(n7)CCNC8)C[C@@H]6C(=O)O)nc4)C5)c4ncnc(N)c34)ccc2o1] | C₅₀H₅₈N₁₆O₉ | [M + H] = 1027.47 | [M + H] = 1027.1 |
| Intermediate B2-28 | | | |
| 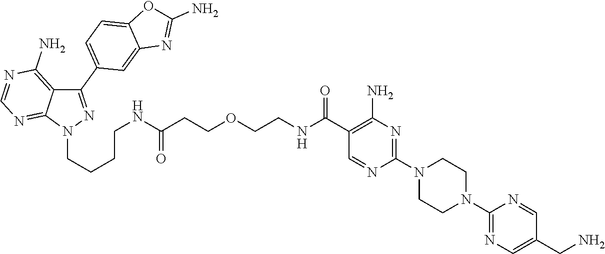 [SMILES: NCc1cnc(N2CCN(c3ncc(C(=O)NCCOCCC(=O)NCCCCn4nc(-c5ccc6oc(N)nc6c5)c5c(N)ncnc54)c(N)n3)CC2)nc1] | C₃₅H₄₃N₁₇O₄ | [M + H] = 766.38 | [M + H] = 766.3 |
| Intermediate B2-29 | | | |
| 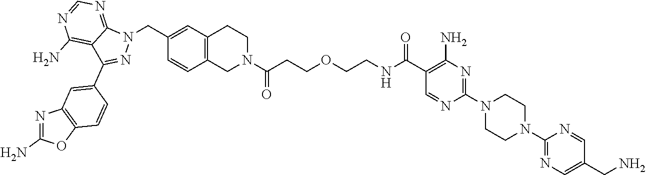 [SMILES: NCc1cnc(N2CCN(c3ncc(C(=O)NCCOCCC(=O)N4CCc5cc(Cn6nc(-c7ccc8oc(N)nc8c7)c7c(N)ncnc76)ccc5C4)c(N)n3)CC2)nc1] | C₄₁H₄₅N₁₇O₄ | [M + H] = 840.39 | [M + H] = 840.4 |
| Intermediate B2-30 | | | |
| 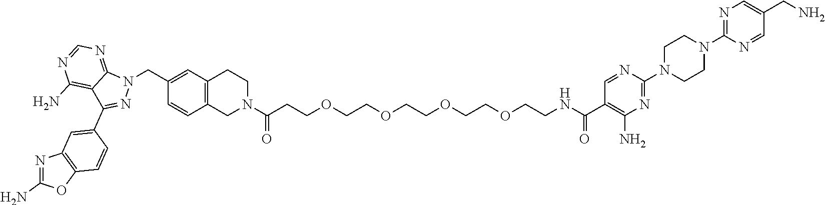 [SMILES: NCc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)c(N)n3)CC2)nc1] | C₄₇H₅₇N₁₇O₇ | [M + H] = 972.47 | [M + H] = 972.5 |
| Intermediate B2-31 | | | |
| 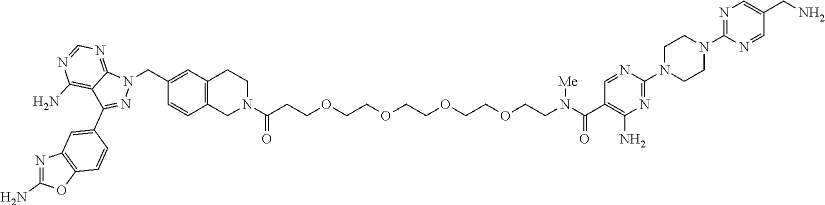 [SMILES: CN(CCOCCOCCOCCOCCC(=O)N1CCc2cc(Cn3nc(-c4ccc5oc(N)nc5c4)c4c(N)ncnc43)ccc2C1)C(=O)c1cnc(N2CCN(c3ncc(CN)cn3)CC2)nc1N] | C₄₈H₅₉N₁₇O₇ | [M + H] = 986.49 | [M + H] = 986.4 |
| Intermediate B2-32 | | | |
| 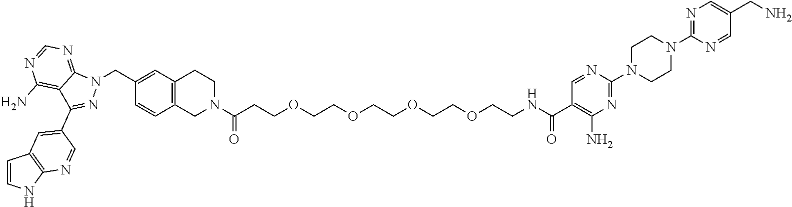 [SMILES: NCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)c(N)n3)CC2)nc1] | C₄₇H₅₇N₁₇O₆ | [M + H] = 956.48 | [M + H] = 956.3 |
| Intermediate B2-33 | | | |
| 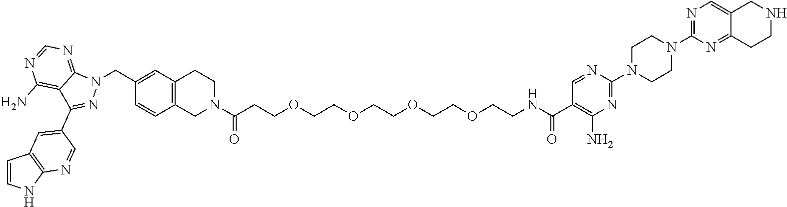 [SMILES: Nc1nc(N2CCN(c3ncc4c(n3)CCNC4)CC2)ncc1C(=O)NCCOCCOCCOCCOCCC(=O)N1CCc2cc(Cn3nc(-c4cnc5[nH]ccc5c4)c4c(N)ncnc43)ccc2C1] | C₄₉H₅₉N₁₇O₆ | [M + H] = 982.49 | [M + H] = 982.2 |
| Intermediate B2-34 | | | |
| 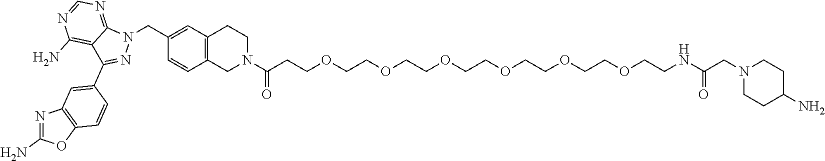 [SMILES: Nc1nc2cc(-c3nn(Cc4ccc5c(c4)CCN(C(=O)CCOCCOCCOCCOCCOCCOCCNC(=O)CN4CCC(N)CC4)C5)c4ncnc(N)c34)ccc2o1] | C₄₄H₆₁N₁₁O₉ | [M + H] = 888.48 | [M + H] = 888.3 |
| Intermediate B2-35 | | | |
| 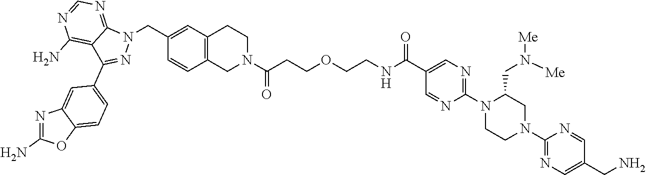 [SMILES: CN(C)C[C@@H]1CN(c2ncc(CN)cn2)CCN1c1ncc(C(=O)NCCOCCC(=O)N2CCc3cc(Cn4nc(-c5ccc6oc(N)nc6c5)c5c(N)ncnc54)ccc3C2)cn1] | C₄₄H₅₁N₁₇O₄ | [M + H] = 882.44 | [M + H] = 882.4 |
| Intermediate B2-36 | | | |
| 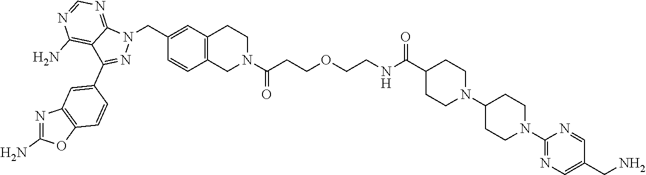 [SMILES: NCc1cnc(N2CCC(N3CCC(C(=O)NCCOCCC(=O)N4CCc5cc(Cn6nc(-c7ccc8oc(N)nc8c7)c7c(N)ncnc76)ccc5C4)CC3)CC2)nc1] | C₄₃H₅₂N₁₄O₄ | [M + H] = 829.44 | [M + H] = 829.3 |
| Intermediate B2-37 | | | |
| 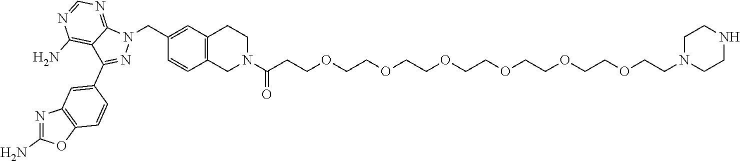 [SMILES: Nc1nc2cc(-c3nn(Cc4ccc5c(c4)CCN(C(=O)CCOCCOCCOCCOCCOCCOCCN4CCNCC4)C5)c4ncnc(N)c34)ccc2o1] | C₄₁H₅₆N₁₀O₈ | [M + H] = 817.44 | [M + H] = 817.2 |
| Intermediate B2-38 | | | |
| 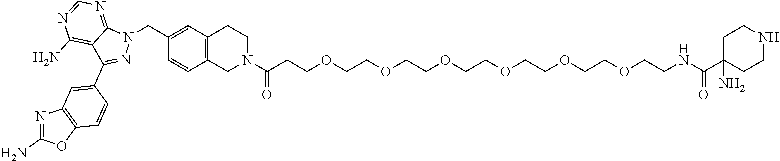 [SMILES: Nc1nc2cc(-c3nn(Cc4ccc5c(c4)CCN(C(=O)CCOCCOCCOCCOCCOCCOCCNC(=O)C4(N)CCNCC4)C5)c4ncnc(N)c34)ccc2o1] | C₄₃H₅₉N₁₁O₉ | [M + H] = 874.46 | [M + H] = 874.3 |
| Intermediate B2-39 | | | |

Following General Procedure 2, but using the appropriate 4-nitrophenyl carbonate containing rapamycin monomer in Table 1 and Intermediates B2 from Tables 9, 11, and 13 and 14, the Series 2 bivalent analogs in Table 15 were synthesized:

TABLE 15
Series 2 Bivalent Compounds:

| | | Calcu- | Ob- |
|---|---|---|---|
| | Molecular | lated | served |
| Structure | Formula | MW | MW |
| 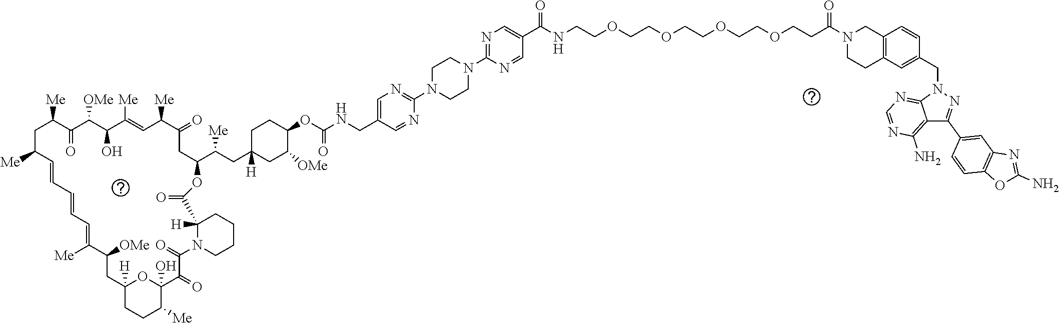 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₃N₁₇O₂₁ | [M + H] = 1896.99 | [M + H] = 1896.7 |
| Example 27 | | | |
| 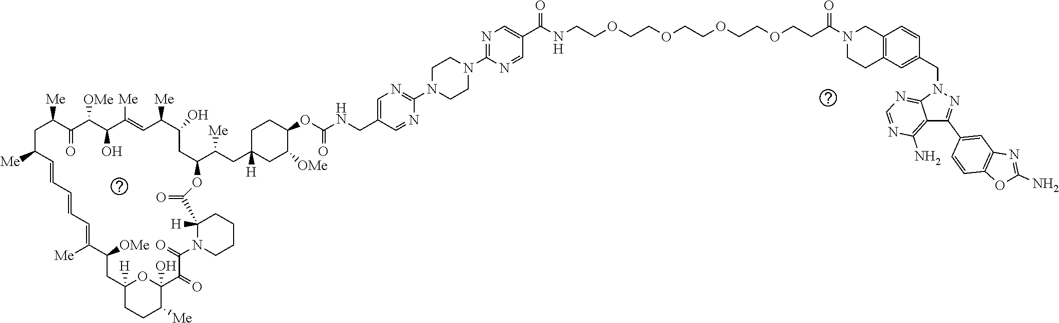 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₉H₁₃₅N₁₇O₂₁ | [M + H] = 1899.01 | [M + H] = 1899.1 |
| Example 28 | | | |
| 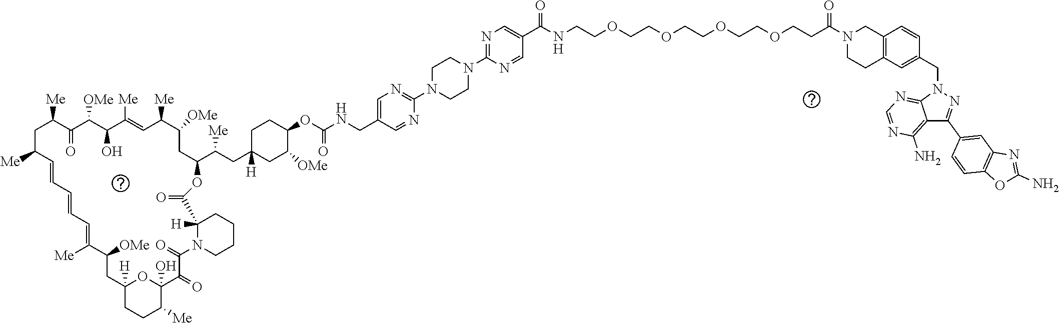 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₇N₁₇O₂₁ | [M + H] = 1913.03 | [M + H] = 1913.0 |
| Example 29 | | | |
| 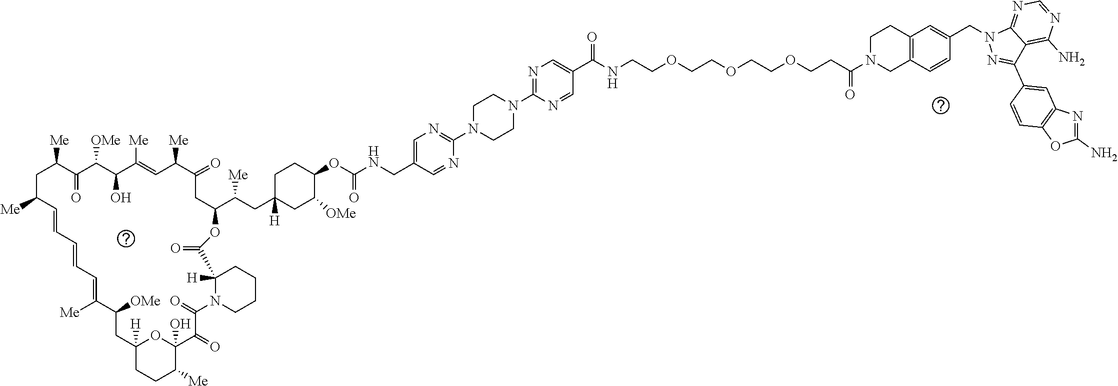 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₉N₁₇O₂₀ | [M + H] = 1852.97 | [M + H] = 1852.9 |
| Example 30 | | | |
| 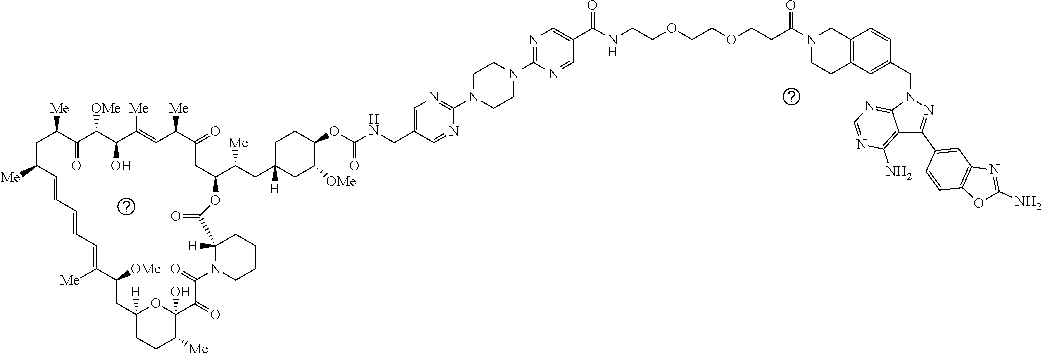 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₂₅N₁₇O₁₉ | [M + H] = 1808.94 | [M + H] = 1809.0 |
| Example 31 | | | |
| 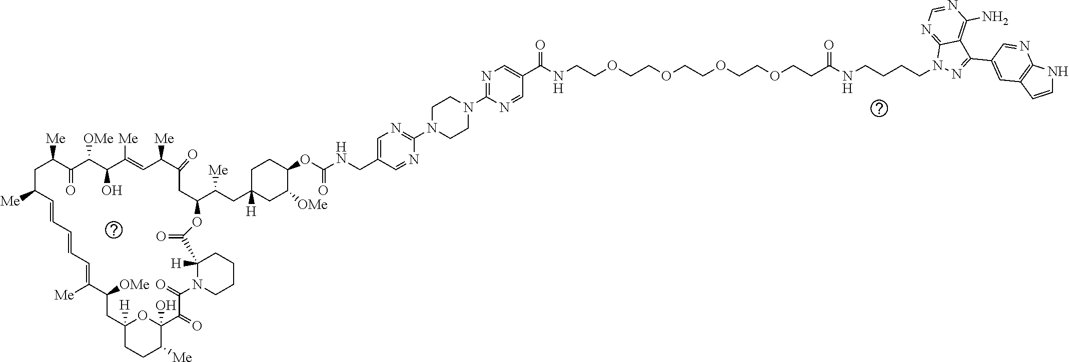 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₃₁N₁₇O₂₀ | [M + H] = 1806.98 | [M + H] = 1806.7 |
| Example 32 | | | |
| 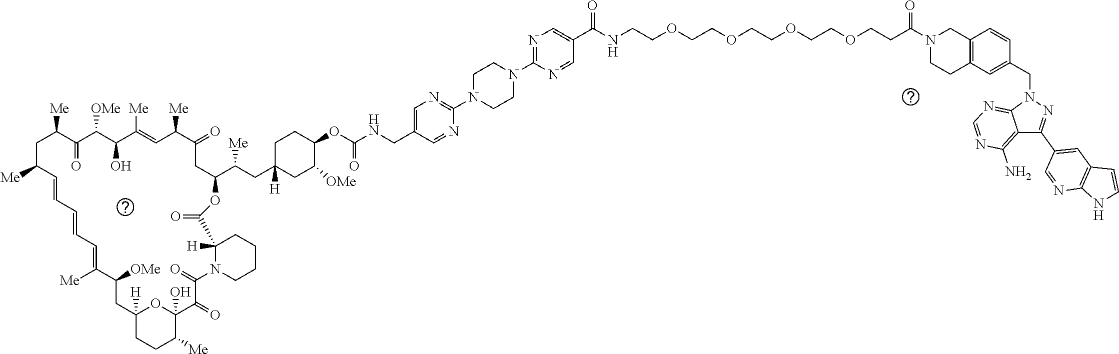 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₃N₁₇O₂₀ | [M + H] = 1881.00 | [M + H] = 1881.0 |
| Example 33 | | | |
| 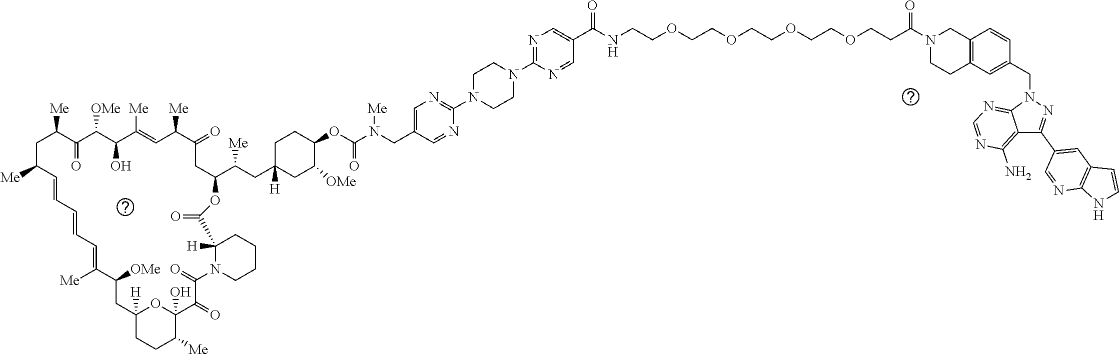 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₅N₁₇O₂₀ | [M + H] = 1895.01 | [M + H] = 1895.1 |
| Example 34 | | | |
| 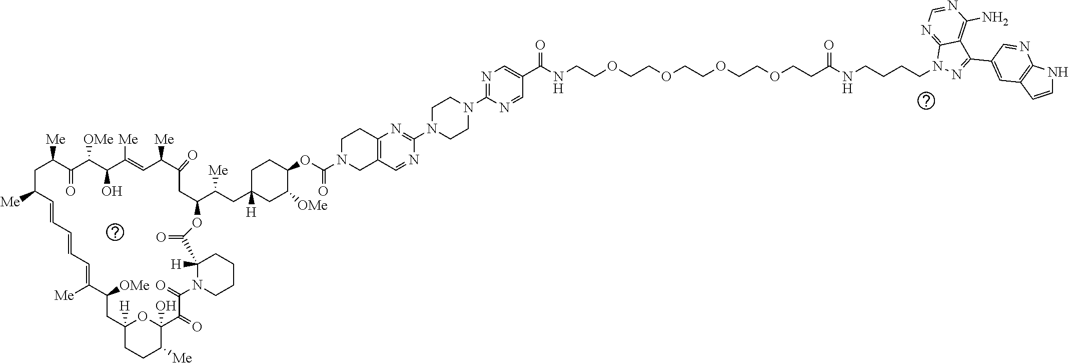 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₃N₁₇O₂₀ | [M + H] = 1833.00 | [M + H] = 1832.9 |
| Example 35 | | | |
| 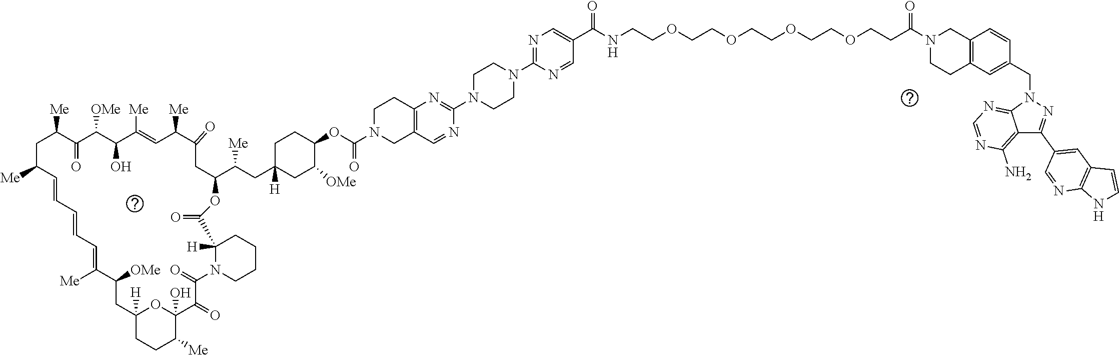 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₃₅N₁₇O₂₀ | [M + H] = 1907.01 | [M + H] = 1907.0 |
| Example 36 | | | |
| 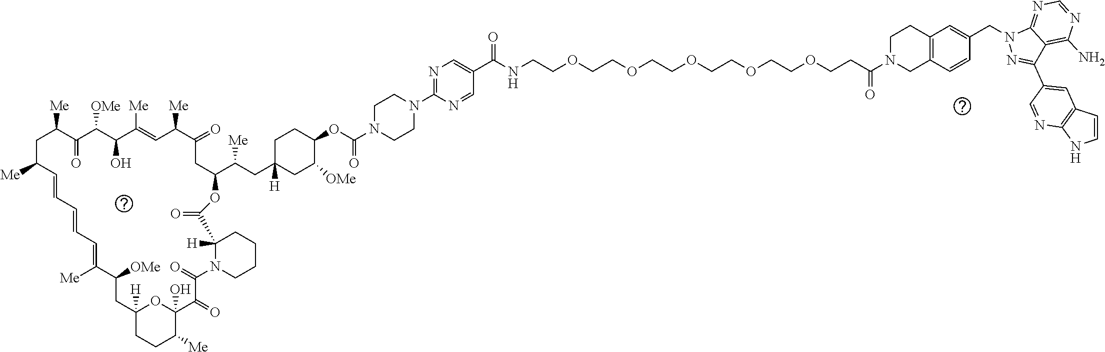 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(c4ncc(C(=O)NCCOCCOCCOCCOCCOCCC(=O)N5CCc6cc(Cn7nc(-c8cnc9[nH]ccc9c8)c8c(N)ncnc87)ccc6C5)cn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₂N₁₄O₂₁ | [M + H] = 1817.98 | [M + H] = 1817.9 |
| Example 37 | | | |
| 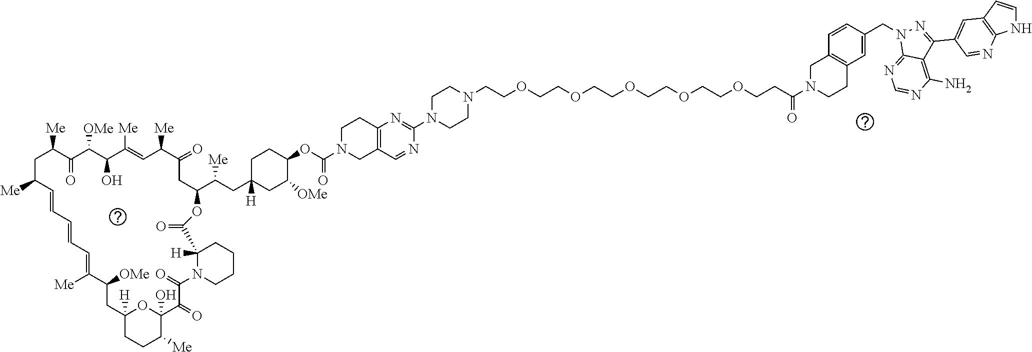 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCOCCOCCOCCOCCC(=O)N6CCc7cc(Cn8nc(-c9cnc%10[nH]ccc%10c9)c9c(N)ncnc98)ccc7C6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₃₆N₁₄O₂₀ | [M + H] = 1830.01 | [M + H] = 1830.1 |
| Example 38 | | | |
| 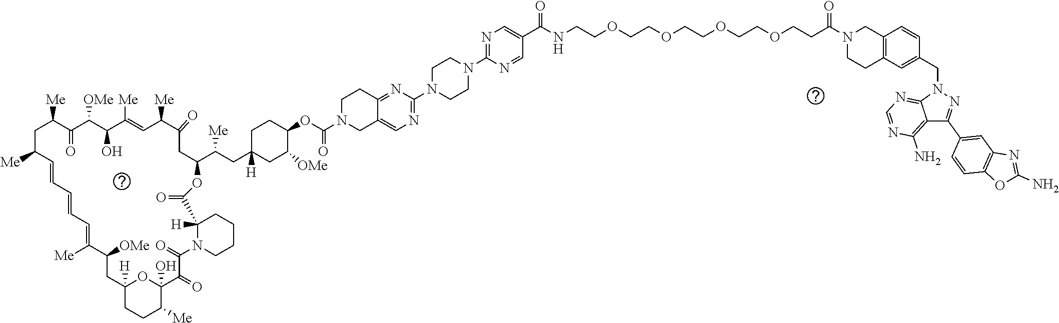 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₃₅N₁₇O₂₁ | [M + H] = 1923.01 | [M + H] = 1923.1 |
| Example 39 | | | |
| 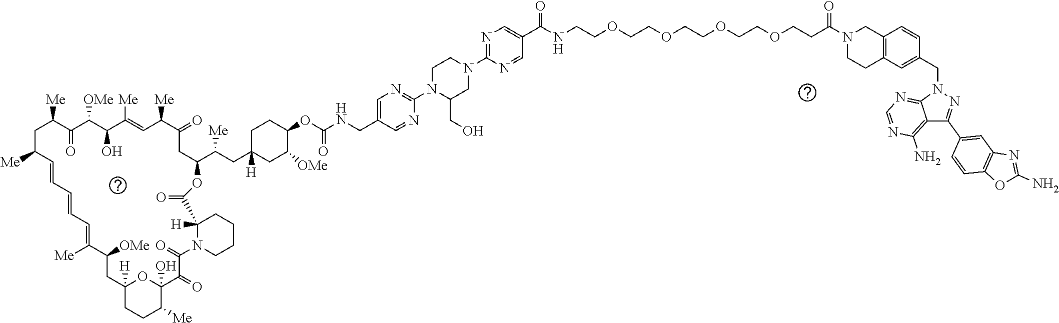 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₅N₁₇O₂₂ | [M + H] = 1927.01 | [M + H] = 1927.0 |
| Example 40 | | | |
| 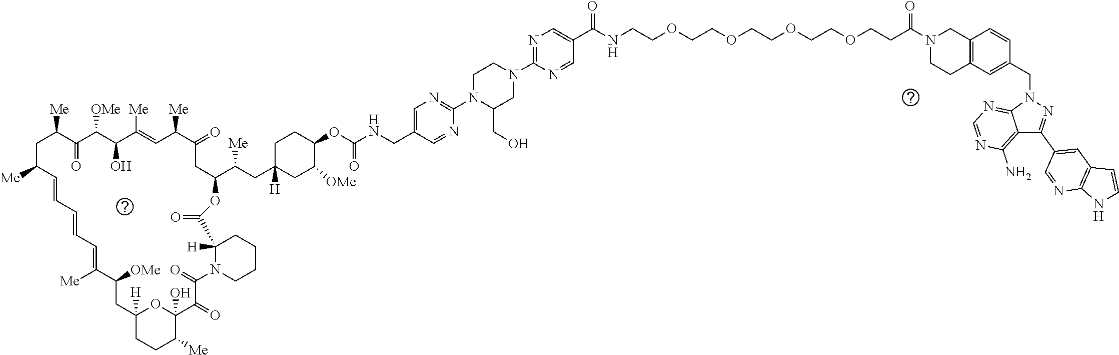 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₅N₁₇O₂₁ | [M + H] = 1911.01 | [M + H] = 1911.0 |
| Example 41 | | | |
| 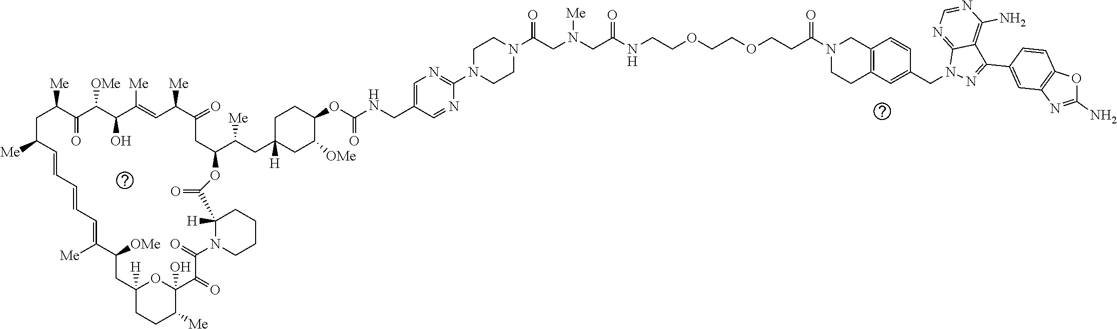 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)N5CCc6cc(Cn7nc(-c8ccc9oc(N)nc9c8)c8c(N)ncnc87)ccc6C5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₀N₁₆O₂₀ | [M + H] = 1815.97 | [M + H] = 1816.0 |
| Example 42 | | | |
| 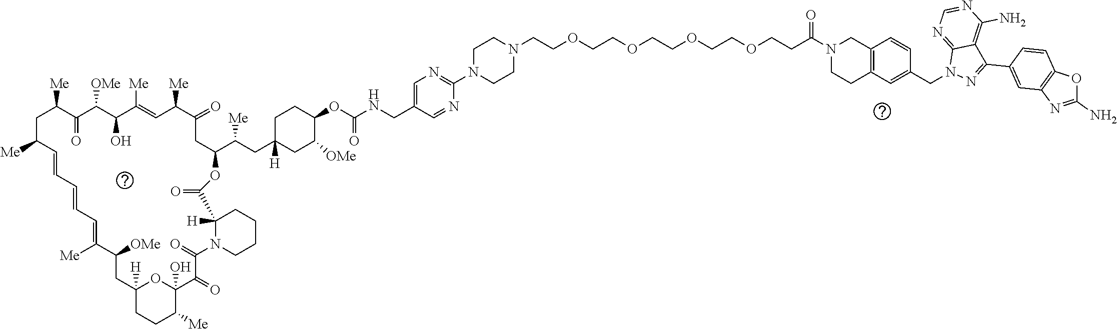 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)N5CCc6cc(Cn7nc(-c8ccc9oc(N)nc9c8)c8c(N)ncnc87)ccc6C5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₀N₁₄O₂₀ | [M + H] = 1775.97 | [M + H] = 1775.9 |
| Example 43 | | | |
| 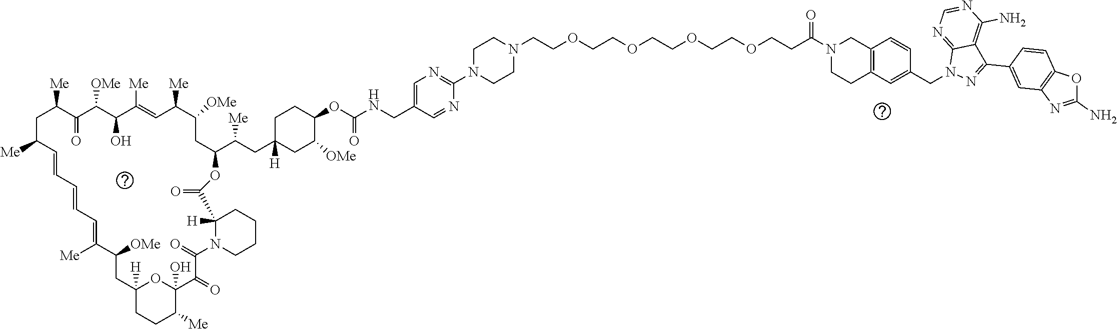 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)N5CCc6cc(Cn7nc(-c8ccc9oc(N)nc9c8)c8c(N)ncnc87)ccc6C5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₅H₁₃₄N₁₄O₂₀ | [M + H] = 1791.99 | [M + H] = 1791.8 |
| Example 44 | | | |
| 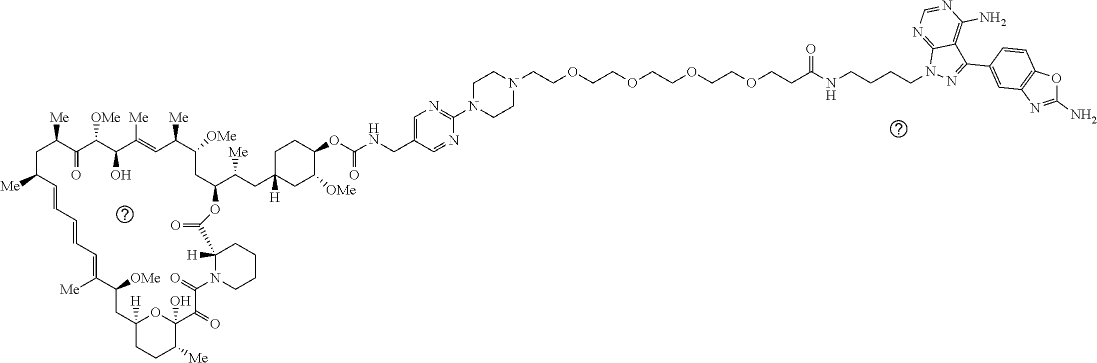 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₈₉H₁₃₂N₁₄O₂₀ | [M + H] = 1717.98 | [M + H] = 1717.9 |
| Example 45 | | | |
| 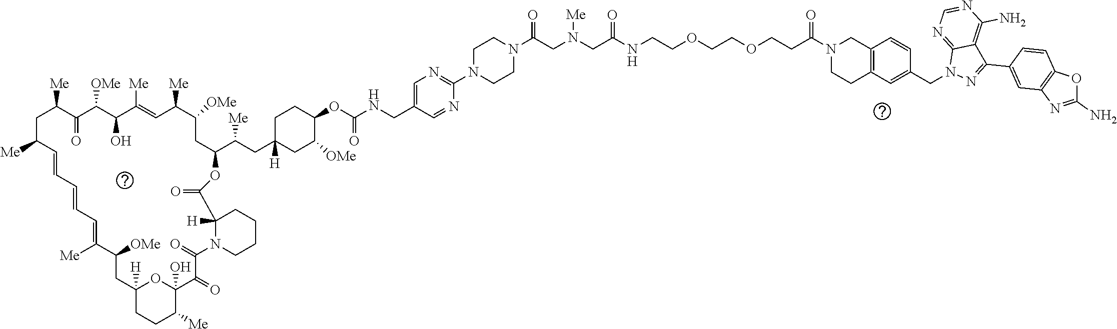 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)N5CCc6cc(Cn7nc(-c8ccc9oc(N)nc9c8)c8c(N)ncnc87)ccc6C5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₃₄N₁₆O₂₀ | [M + H] = 1832.00 | [M + H] = 1832.0 |
| Example 103 | | | |
| 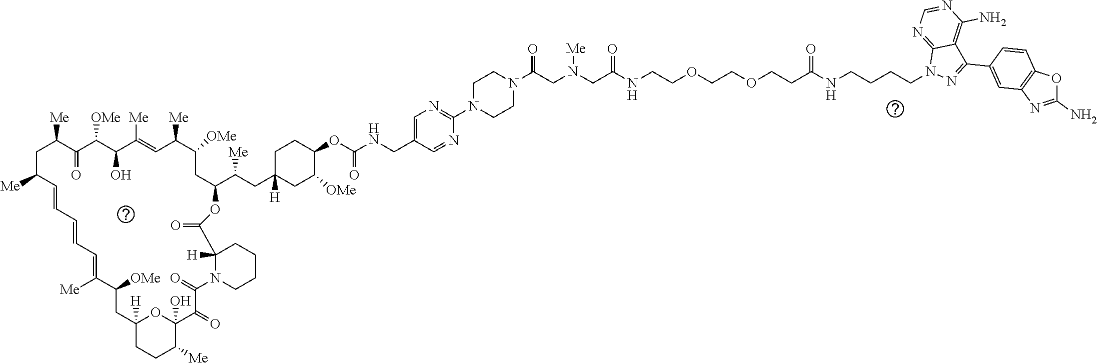 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₀H₁₃₂N₁₆O₂₀ | [M + H] = 1757.99 | [M + H] = 1757.9 |
| Example 104 | | | |
| 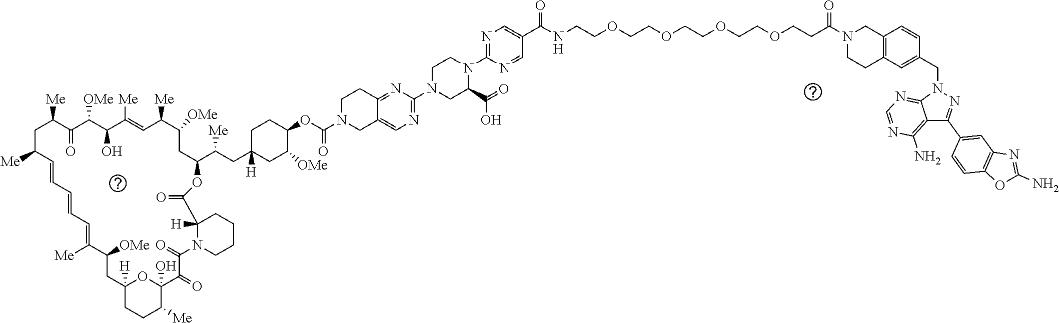 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)[C@@H](C(=O)O)C5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₃H₁₃₉N₁₇O₂₃ | [M + H] = 1983.03 | [M + H] = 1983.0 |
| Example 105 | | | |
| 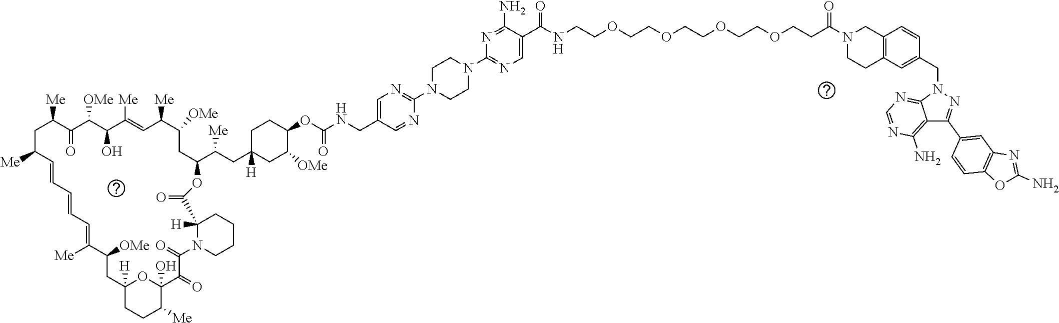 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)c(N)n5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₈N₁₈O₂₁ | [M + H] = 1928.04 | [M + H] = 1927.9 |
| Example 106 | | | |
| 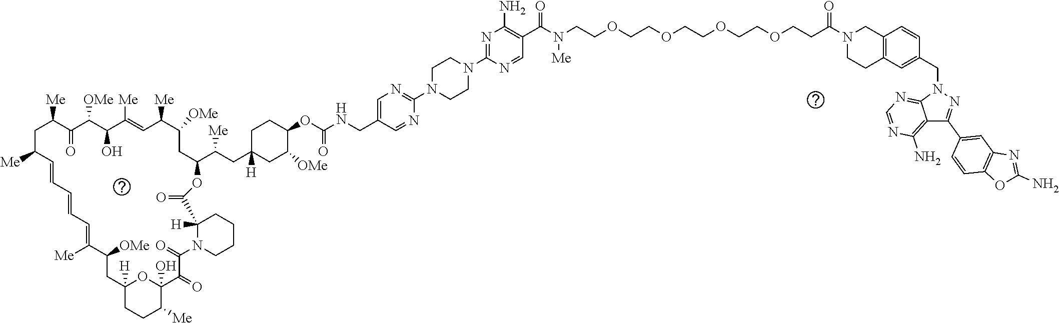 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N(C)CCOCCOCCOCCOCCC(=O)N6CCc7cc(Cn8nc(-c9ccc%10oc(N)nc%10c9)c9c(N)ncnc98)ccc7C6)c(N)n5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₁H₁₄₀N₁₈O₂₁ | [M + H] = 1942.05 | [M + H] = 1942.1 |
| Example 107 | | | |
| 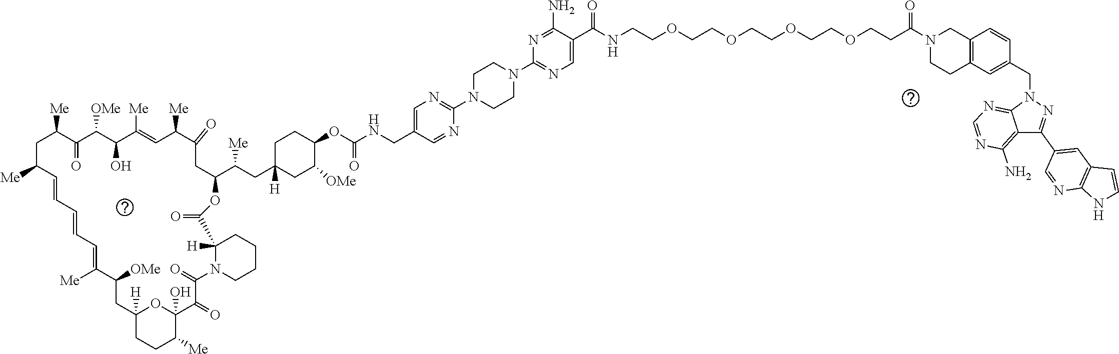 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)c(N)n5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₄N₁₈O₂₀ | [M + H] = 1896.01 | [M + H] = 1896.0 |
| Example 108 | | | |
| 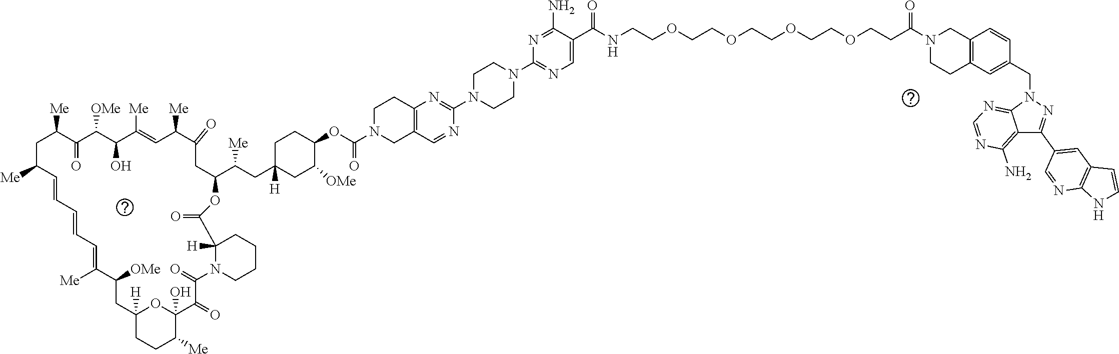 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)c(N)n6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₃₆N₁₈O₂₀ | [M + H] = 1922.03 | [M + H] = 1922.1 |
| Example 109 | | | |
| 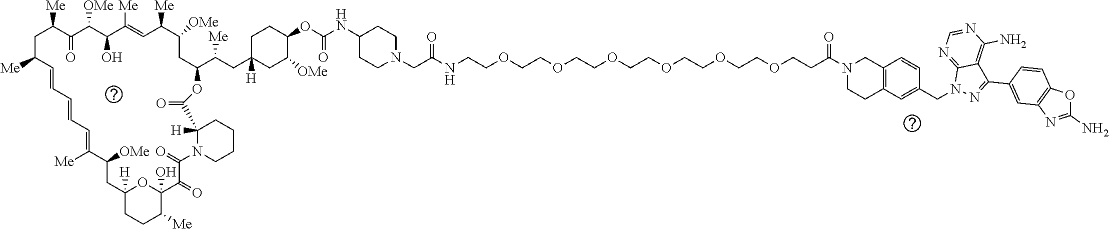 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NC3CCN(CC(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₄₀N₁₂O₂₃ | [M + H] = 1830.02 | [M + H] = 1829.9 |
| Example 110 | | | |
| 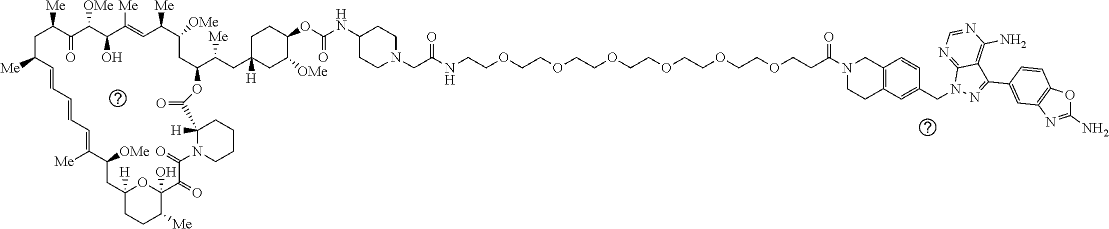 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NC3CCN(CC(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₇H₁₄₂N₁₂O₂₃ | [M + H] = 1844.04 | [M + H] = 1843.9 |
| Example 111 | | | |
| 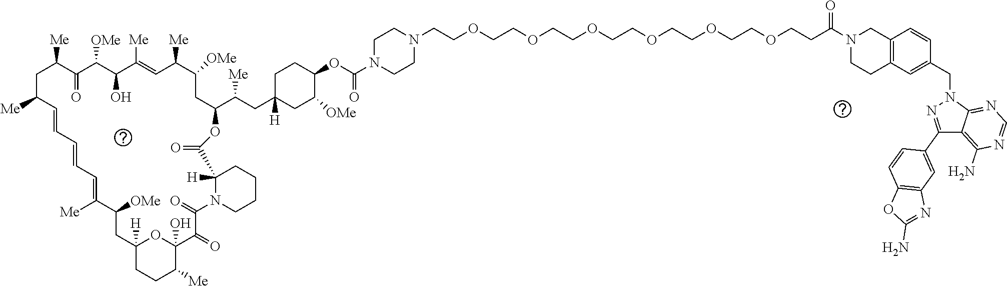 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(CCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₃H₁₃₅N₁₁O₂₂ | [M + H] = 1758.99 | [M + H] = 1758.9 |
| Example 149 | | | |
| 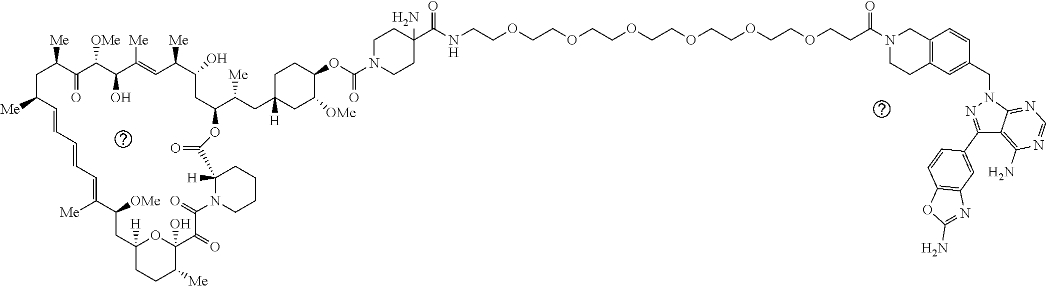 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCC(N)(C(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₃₈N₁₂O₂₃ | [M + H] = 1816.01 | [M + H] = 1815.9 |
| Example 150 | | | |

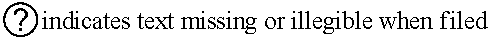

Following General Procedure 1, but using the appropriate amine containing active site inhibitors in Table 2 and amine containing pre-linkers in Table 4, the Intermediates C1 in Table 16 were prepared:

TABLE 16
Additional amines prepared

| | Molecular | Calculated | Observed |
|---|---|---|---|
| Structure | Formula | MW | MW |
| 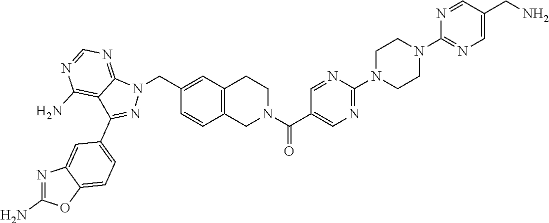 [SMILES: NCc1cnc(N2CCN(c3ncc(C(=O)N4CCc5cc(Cn6nc(-c7ccc8oc(N)nc8c7)c7c(N)ncnc76)ccc5C4)cn3)CC2)nc1] | C₃₆H₃₅N₁₅O₂ | [M + H] = 710.32 | [M + H] = 710.2 |
| Intermediate C1-1 | | | |
| 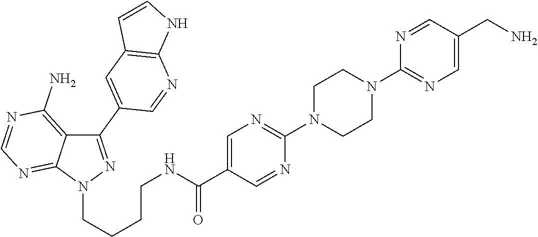 [SMILES: NCc1cnc(N2CCN(c3ncc(C(=O)NCCCCn4nc(-c5cnc6[nH]ccc6c5)c5c(N)ncnc54)cn3)CC2)nc1] | C₃₀H₃₃N₁₅O | [M + H] = 620.31 | [M + H] = 620.2 |
| Intermediate C1-2 | | | |
| 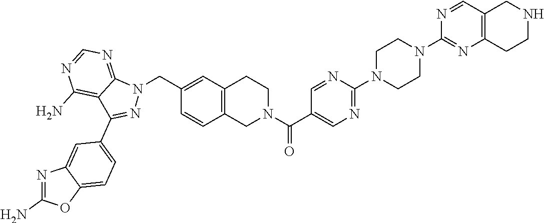 [SMILES: Nc1nc2cc(-c3nn(Cc4ccc5c(c4)CCN(C(=O)c4cnc(N6CCN(c7ncc8c(n7)CCNC8)CC6)nc4)C5)c4ncnc(N)c34)ccc2o1] | C₃₈H₃₇N₁₅O₂ | [M + H] = 736.34 | [M + H] = 736.2 |
| Intermediate C1-3 | | | |
| 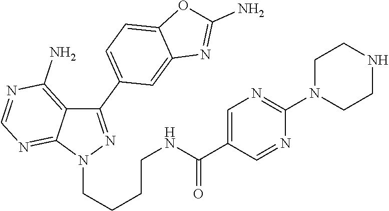 [SMILES: Nc1nc2cc(-c3nn(CCCCNC(=O)c4cnc(N5CCNCC5)nc4)c4ncnc(N)c34)ccc2o1] | C₂₅H₂₈N₁₂O₂ | [M + H] = 529.26 | [M + H] = 529.5 |
| Intermediate C1-4 | | | |
| 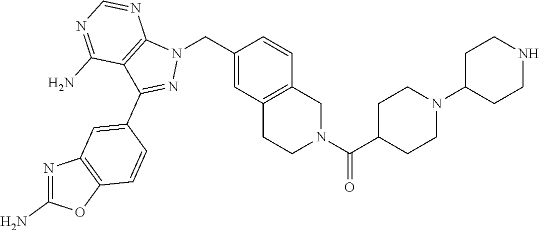 [SMILES: Nc1nc2cc(-c3nn(Cc4ccc5c(c4)CCN(C(=O)C4CCN(C6CCNCC6)CC4)C5)c4ncnc(N)c34)ccc2o1] | C₃₃H₃₈N₁₀O₂ | [M + H] = 607.33 | [M + H] = 607.3 |
| Intermediate C1-5 | | | |
| 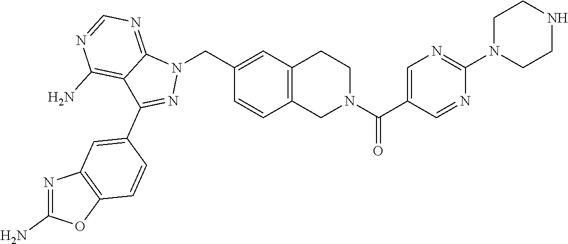 [SMILES: Nc1nc2cc(-c3nn(Cc4ccc5c(c4)CCN(C(=O)c4cnc(N6CCNCC6)nc4)C5)c4ncnc(N)c34)ccc2o1] | C₃₁H₃₀N₁₂O₂ | [M + H] = 603.27 | [M + H] = 603.3 |
| Intermediate C1-6 | | | |
| 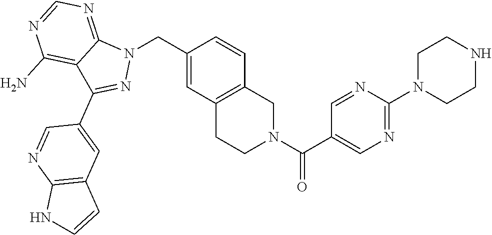 [SMILES: Nc1ncnc2c1c(-c1cnc3[nH]ccc3c1)nn2Cc1ccc2c(c1)CCN(C(=O)c1cnc(N3CCNCC3)nc1)C2] | C₃₁H₃₀N₁₂O | [M + H] = 587.28 | [M + H] = 587.3 |
| Intermediate C1-7 | | | |
| 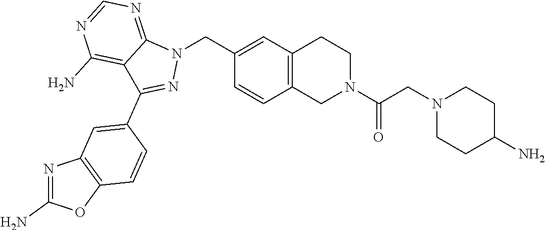 [SMILES: Nc1nc2cc(-c3nn(Cc4ccc5c(c4)CCN(C(=O)CN4CCC(N)CC4)C5)c4ncnc(N)c34)ccc2o1] | C₂₉H₃₂N₁₀O₂ | [M + H] = 553.28 | [M + H] = |
| Intermediate C1-8 | | | |

Following General Procedure 1, but using the PEG carboxylic acids and Intermediates C1 in Table 16, the Intermediates C2 in Table 17 were prepared:

TABLE 17
Additional amines prepared

| | | Calcu- | Ob- |
|---|---|---|---|
| | Molecular | lated | served |
| Structure | Formula | MW | MW |
| 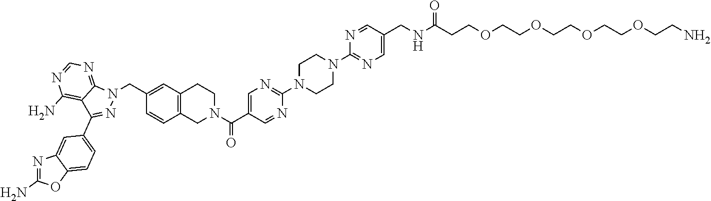 [SMILES: NCCOCCOCCOCCOCCC(=O)NCc1cnc(N2CCN(c3ncc(C(=O)N4CCc5cc(Cn6nc(-c7ccc8oc(N)nc8c7)c7c(N)ncnc76)ccc5C4)cn3)CC2)nc1] | C₄₇H₅₆N₁₆O₇ | [M + H] = 957.46 | [M + H] = 957.7 |
| Intermediate C2-1 | | | |
| 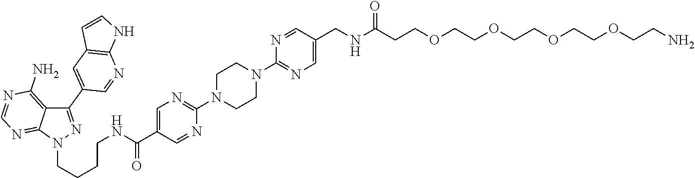 [SMILES: NCCOCCOCCOCCOCCC(=O)NCc1cnc(N2CCN(c3ncc(C(=O)NCCCCn4nc(-c5cnc6[nH]ccc6c5)c5c(N)ncnc54)cn3)CC2)nc1] | C₄₁H₅₄N₁₆O₆ | [M + H] = 867.45 | [M + H] = 867.2 |
| Intermediate C2-2 | | | |
| 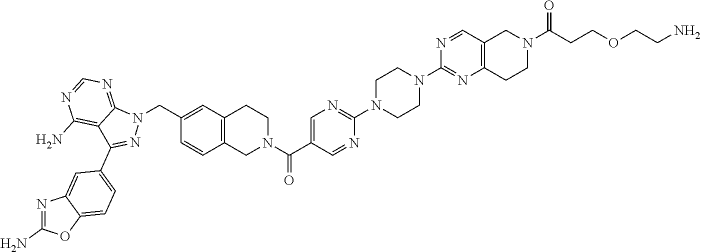 [SMILES: NCCOCCC(=O)N1CCc2nc(N3CCN(c4ncc(C(=O)N5CCc6cc(Cn7nc(-c8ccc9oc(N)nc9c8)c8c(N)ncnc87)ccc6C5)cn4)CC3)ncc2C1] | C₄₃H₄₆N₁₆O₄ | [M + H] = 851.40 | [M + H] = 851.2 |
| Intermediate C2-3 | | | |
| 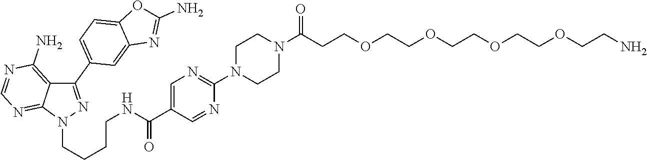 [SMILES: NCCOCCOCCOCCOCCC(=O)N1CCN(c2ncc(C(=O)NCCCCn3nc(-c4ccc5oc(N)nc5c4)c4c(N)ncnc43)cn2)CC1] | C₃₆H₄₉N₁₃O₇ | [M + H] = 776.40 | [M + H] = 776.3 |
| Intermediate C2-4 | | | |
| 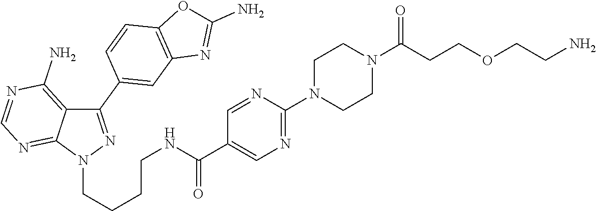 [SMILES: NCCOCCC(=O)N1CCN(c2ncc(C(=O)NCCCCn3nc(-c4ccc5oc(N)nc5c4)c4c(N)ncnc43)cn2)CC1] | C₃₀H₃₇N₁₃O₄ | [M + H] = 644.32 | [M + H] = 644.3 |
| Intermediate C2-5 | | | |
| 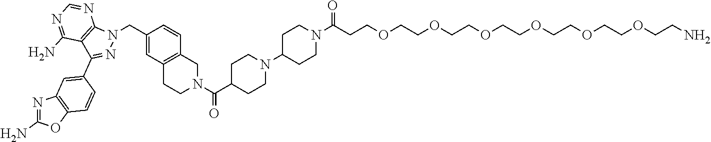 [SMILES: NCCOCCOCCOCCOCCOCCOCCC(=O)N1CCC(N2CCC(C(=O)N3CCc4cc(Cn5nc(-c6ccc7oc(N)nc7c6)c6c(N)ncnc65)ccc4C3)CC2)CC1] | C₄₈H₆₇N₁₁O₉ | [M + H] = 942.52 | [M + H] = 943.2 |
| Intermediate C2-6 | | | |
| 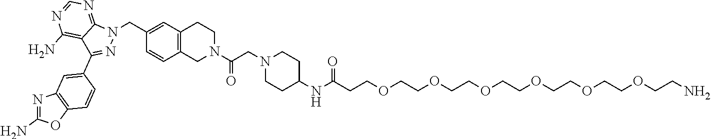 [SMILES: NCCOCCOCCOCCOCCOCCOCCC(=O)NC1CCN(CC(=O)N2CCc3cc(Cn4nc(-c5ccc6oc(N)nc6c5)c5c(N)ncnc54)ccc3C2)CC1] | C₄₄H₆₁N₁₁O₉ | [M + H] = 888.48 | [M + H] = 888.3 |
| Intermediate C2-7 | | | |

Following General Procedure 2, but using the appropriate 4-nitrophenyl carbonate containing rapamycin monomer in Table 1 and Intermediates C2 from Table 17, the Series 3 bivalent analogs in Table 18 were synthesized:

TABLE 18
Series 3 Bivalent Compounds

| | | Calcu- | Ob- |
|---|---|---|---|
| | Molecular | lated | served |
| Structure | Formula | MW | MW |
| 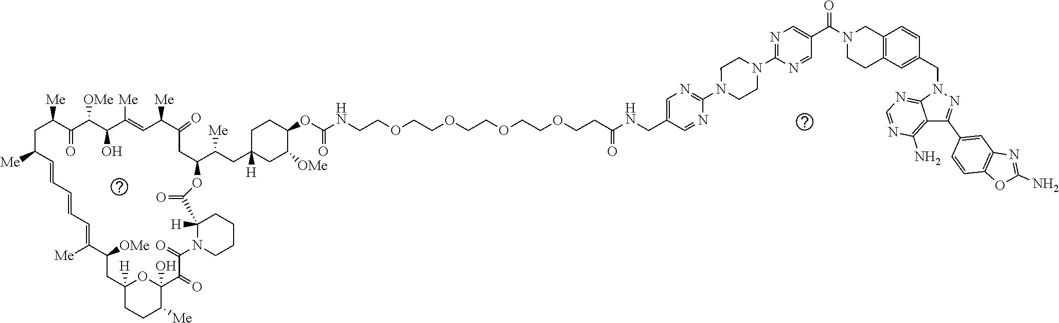 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₃N₁₇O₂₁ | [M + H] = 1896.99 | [M + H] = 1897.3 |
| Example 46 | | | |
| 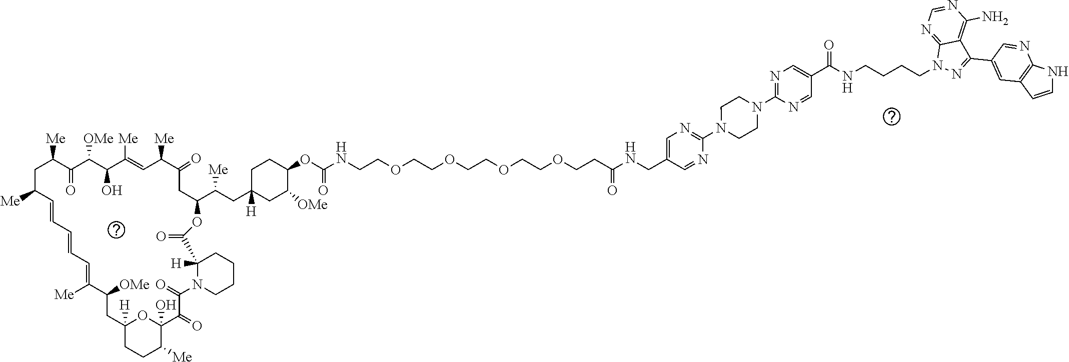 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₃₁N₁₇O₂₀ | [M + H] = 1806.98 | [M + H] = 1806.8 |
| Example 47 | | | |
| 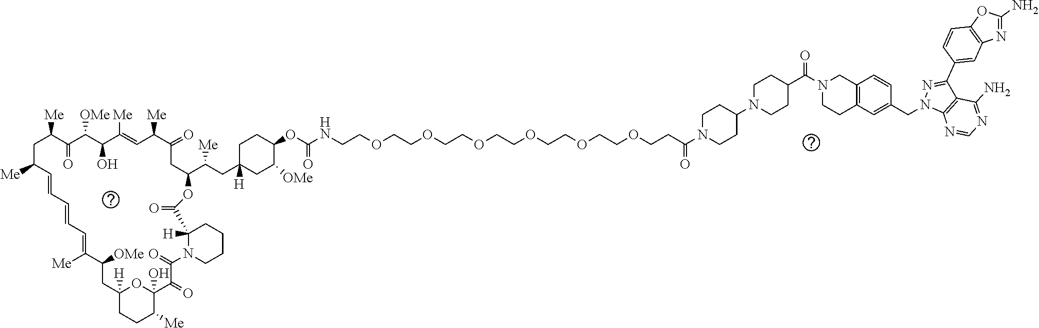 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCC(N4CCC(C(=O)N5CCc6cc(Cn7nc(-c8ccc9oc(N)nc9c8)c8c(N)ncnc87)ccc6C5)CC4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₄₄N₁₂O₂₃ | [M + H] = 1882.06 | [M + H] = 1882.1 |
| Example 48 | | | |
| 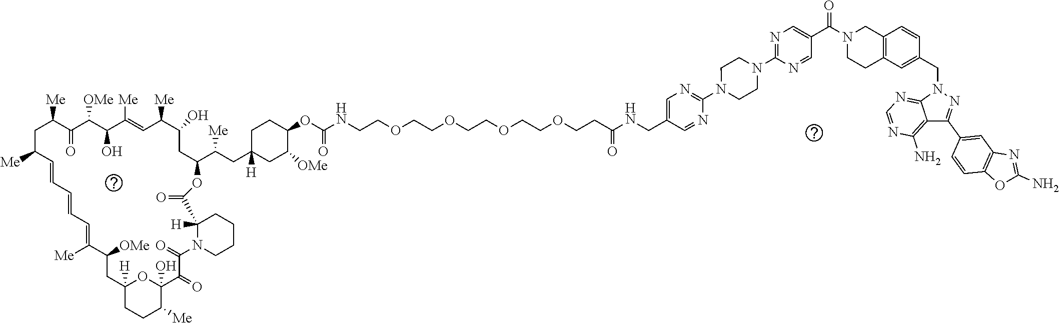 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₉H₁₃₅N₁₇O₂₁ | [M + H] = 1899.01 | [M + H] = 1899.1 |
| Example 49 | | | |
| 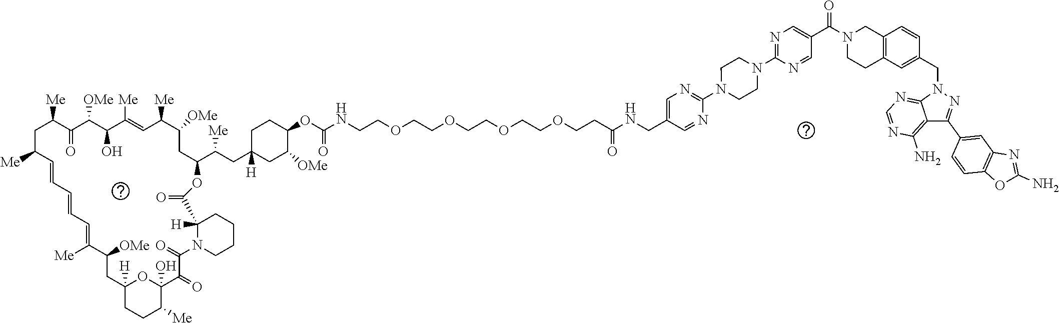 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₇N₁₇O₂₁ | [M + H] = 1913.03 | [M + H] = 1913.1 |
| Example 50 | | | |
| 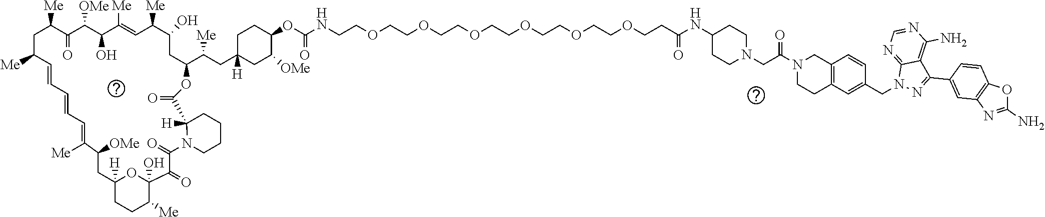 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)NC3CCN(CC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₆H₁₄₀N₁₂O₂₃ | [M + H] = 1830.02 | [M + H] = 1829.9 |
| Example 112 | | | |
| 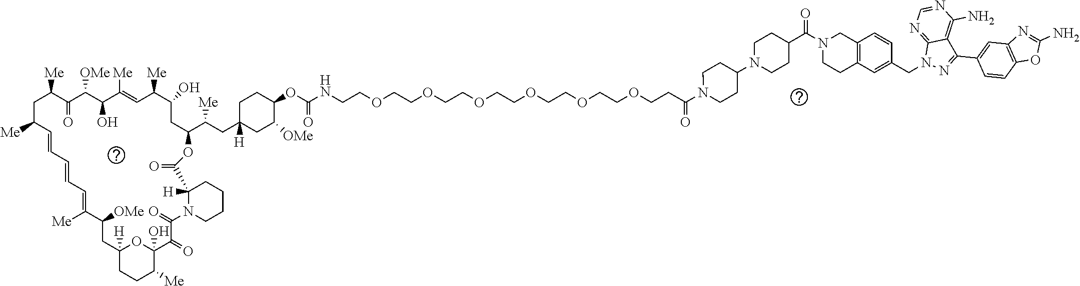 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCC(N4CCC(C(=O)N5CCc6cc(Cn7nc(-c8ccc9oc(N)nc9c8)c8c(N)ncnc87)ccc6C5)CC4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₁₀₀H₁₄₆N₁₂O₂₃ | [M + H] = 1884.07 | [M + H] = 1883.7 |
| Example 113 | | | |

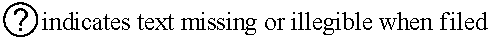

Following General Procedure 1, but using the appropriate Intermediates C2 in Table 17 and amine containing pre-linkers in Table 4, the Intermediates D1 in Table 19 were prepared:

TABLE 19
Additional amines prepared

| | | Calcu- | Ob- |
|---|---|---|---|
| | Molecular | lated | served |
| Structure | Formula | MW | MW |
| 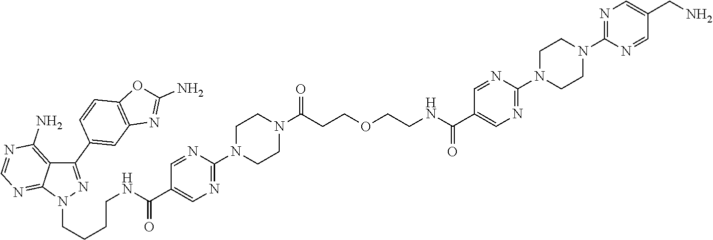 [SMILES: NCc1cnc(N2CCN(c3ncc(C(=O)NCCOCCC(=O)N4CCN(c5ncc(C(=O)NCCCCn6nc(-c7ccc8oc(N)nc8c7)c7c(N)ncnc76)cn5)CC4)cn3)CC2)nc1] | C₄₄H₅₂N₂₀O₅ | [M + H] = 941.45 | [M + H] = 941.5 |
| Intermediate D1-1 | | | |

Following General Procedure 1, but using the appropriate amine containing active site inhibitors in Table 2 and amine containing pre-linkers in Table 4, the Intermediates D1 in Table 20 were prepared:

TABLE 20
Additional amines prepared

| | | Calcu- | Ob- |
|---|---|---|---|
| | Molecular | lated | served |
| Structure | Formula | MW | MW |
| 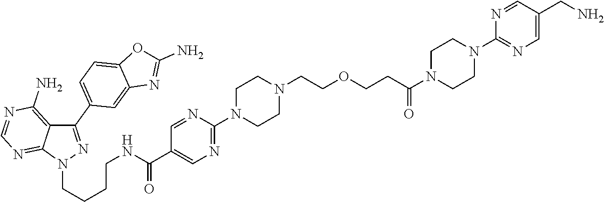 [SMILES: NCc1cnc(N2CCN(C(=O)CCOCCN3CCN(c4ncc(C(=O)NCCCCn5nc(-c6ccc7oc(N)nc7c6)c6c(N)ncnc65)cn4)CC3)CC2)nc1] | C₃₉H₄₉N₁₇O₄ | [M + H] = 820.43 | [M + H] = 820.4 |
| Intermediate D1-2 | | | |
| 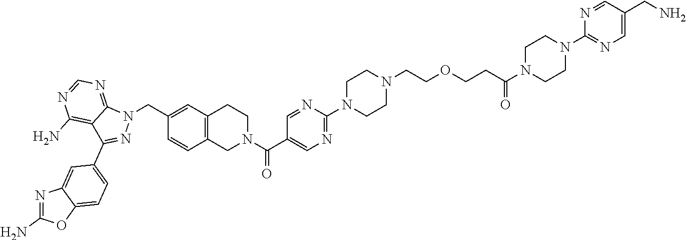 [SMILES: NCc1cnc(N2CCN(C(=O)CCOCCN3CCN(c4ncc(C(=O)N5CCc6cc(Cn7nc(-c8ccc9oc(N)nc9c8)c8c(N)ncnc87)ccc6C5)cn4)CC3)CC2)nc1] | C₄₅H₅₁N₁₇O₄ | [M + H] = 894.44 | [M + H] = 894.4 |
| Intermediate D1-3 | | | |
| 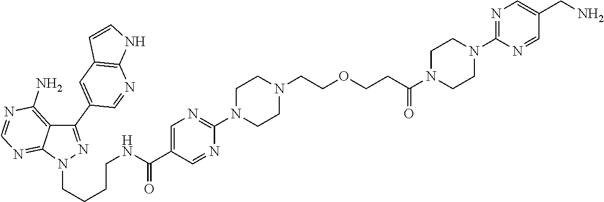 [SMILES: NCc1cnc(N2CCN(C(=O)CCOCCN3CCN(c4ncc(C(=O)NCCCCn5nc(-c6cnc7[nH]ccc7c6)c6c(N)ncnc65)cn4)CC3)CC2)nc1] | C₃₉H₄₉N₁₇O₃ | [M + H] = 804.43 | [M + H] = 804.4 |
| Intermediate D1-4 | | | |
| 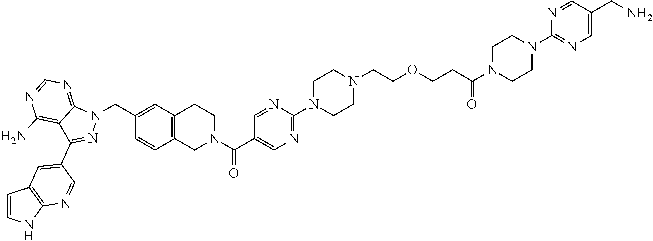 [SMILES: NCc1cnc(N2CCN(C(=O)CCOCCN3CCN(c4ncc(C(=O)N5CCc6cc(Cn7nc(-c8cnc9[nH]ccc9c8)c8c(N)ncnc87)ccc6C5)cn4)CC3)CC2)nc1] | C₄₅H₅₁N₁₇O₃ | [M + H] = 878.45 | [M + H] = 878.4 |
| Intermediate D1-5 | | | |
| 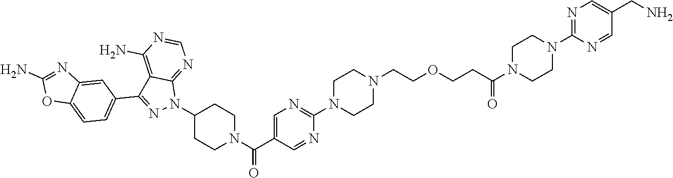 [SMILES: NCc1cnc(N2CCN(C(=O)CCOCCN3CCN(c4ncc(C(=O)N5CCC(n6nc(-c7ccc8oc(N)nc8c7)c7c(N)ncnc76)CC5)cn4)CC3)CC2)nc1] | C₄₀H₄₉N₁₇O₄ | [M + H] = 832.43 | [M + H = 832.4 |
| Intermediate D1-6 | | | |
| 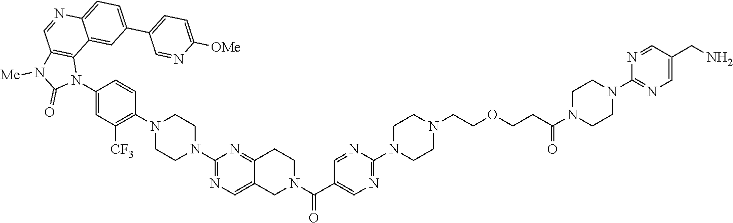 [SMILES: COc1ccc(-c2ccc3ncc4c(c3c2)n(-c2ccc(N3CCN(c5ncc6c(n5)CCN(C(=O)c5cnc(N7CCN(CCOCCC(=O)N8CCN(c9ncc(CN)cn9)CC8)CC7)nc5)C6)CC3)c(C(F)(F)F)c2)c(=O)n4C)cn1] | C₅₈H₆₃F₃N₁₈O₅ | [M + H] = 1149.53 | [M + H] = 1149.4 |
| Intermediate D1-7 | | | |
| 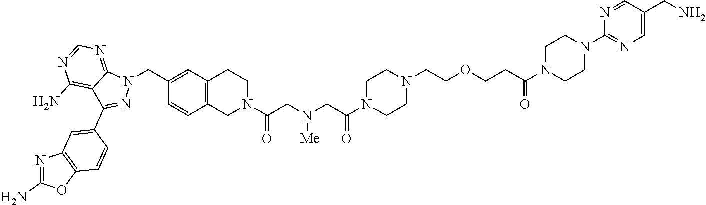 [SMILES: CN(CC(=O)N1CCN(CCOCCC(=O)N2CCN(c3ncc(CN)cn3)CC2)CC1)CC(=O)N1CCc2cc(Cn3nc(-c4ccc5oc(N)nc5c4)c4c(N)ncnc43)ccc2C1] | C₄₅H₅₆N₁₆O₅ | [M + H] = 901.47 | [M + H] = 901.4 |
| Intermediate D1-8 | | | |
| 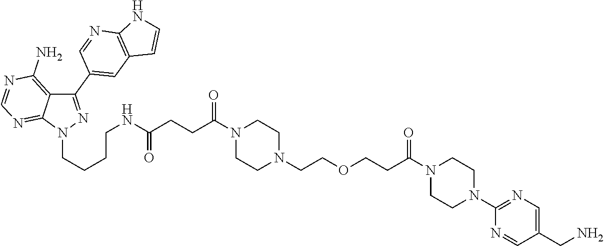 [SMILES: NCc1cnc(N2CCN(C(=O)CCOCCN3CCN(C(=O)CCC(=O)NCCCCn4nc(-c5cnc6[nH]ccc6c5)c5c(N)ncnc54)CC3)CC2)nc1] | C₃₈H₅₁N₁₅O₄ | [M + H] = 782.43 | [M + H] = 782.4 |
| Intermediate D1-9 | | | |
| 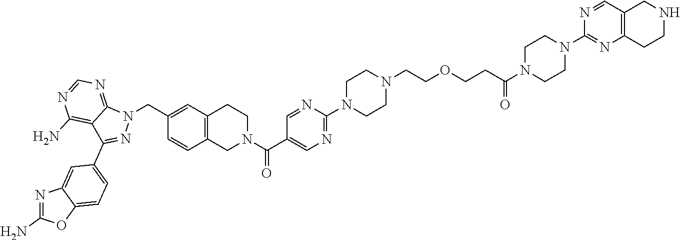 [SMILES: Nc1nc2cc(-c3nn(Cc4ccc5c(c4)CCN(C(=O)c4cnc(N6CCN(CCOCCC(=O)N7CCN(c8ncc9c(n8)CCNC9)CC7)CC6)nc4)C5)c4ncnc(N)c34)ccc2o1] | C₄₇H₅₃N₁₇O₄ | [M + H] = 920.46 | [M + H] = 920.4 |
| Intermediate D1-10 | | | |
| 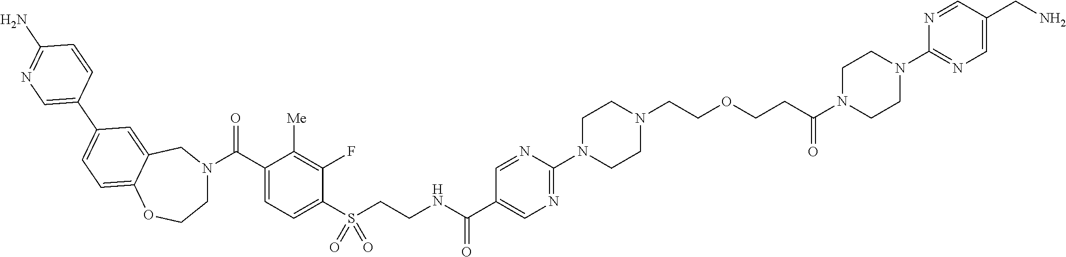 [SMILES: Cc1c(C(=O)N2CCOc3ccc(-c4ccc(N)nc4)cc3C2)ccc(S(=O)(=O)CCNC(=O)c2cnc(N3CCN(CCOCCC(=O)N4CCN(c5ncc(CN)cn5)CC4)CC3)nc2)c1F] | C₄₇H₅₆FN₁₃O₇S | [M + H] = 966.42 | [M + H] = 966.3 |
| Intermediate D1-11 | | | |
| 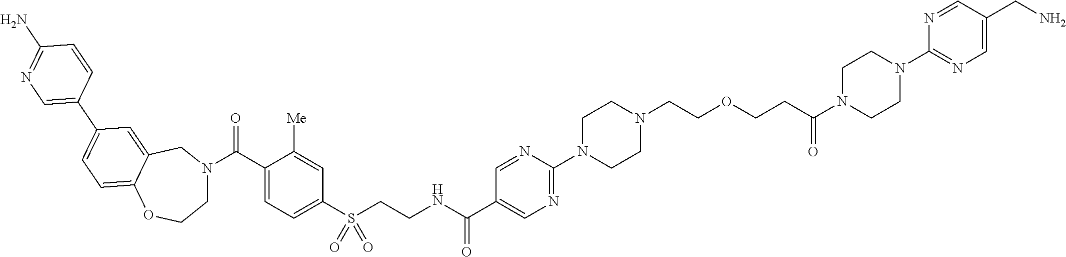 [SMILES: Cc1cc(S(=O)(=O)CCNC(=O)c2cnc(N3CCN(CCOCCC(=O)N4CCN(c5ncc(CN)cn5)CC4)CC3)nc2)ccc1C(=O)N1CCOc2ccc(-c3ccc(N)nc3)cc2C1] | C₄₇H₅₇N₁₃O₇S | [M + H] = 948.43 | [M + H] = 948.4 |
| Intermediate D1-12 | | | |
| 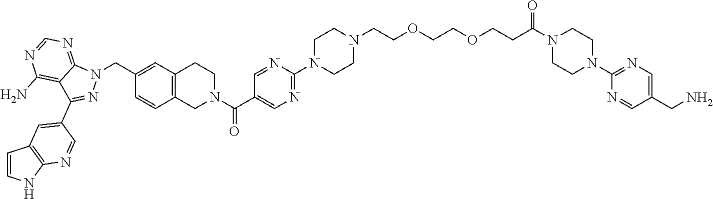 [SMILES: NCc1cnc(N2CCN(C(=O)CCOCCOCCN3CCN(c4ncc(C(=O)N5CCc6cc(Cn7nc(-c8cnc9[nH]ccc9c8)c8c(N)ncnc87)ccc6C5)cn4)CC3)CC2)nc1] | C₄₇H₅₅N₁₇O₄ | [M + H] = 922.47 | [M + H] = 922.4 |
| Intermediate D1-13 | | | |
| 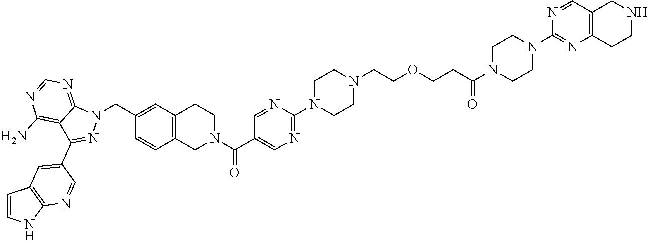 [SMILES: Nc1ncnc2c1c(-c1cnc3[nH]ccc3c1)nn2Cc1ccc2c(c1)CCN(C(=O)c1cnc(N3CCN(CCOCCC(=O)N4CCN(c5ncc6c(n5)CCNC6)CC4)CC3)nc1)C2] | C₄₇H₅₃N₁₇O₃ | [M + H] = 904.46 | [M + H] = 904.4 |
| Intermediate D1-14 | | | |
| 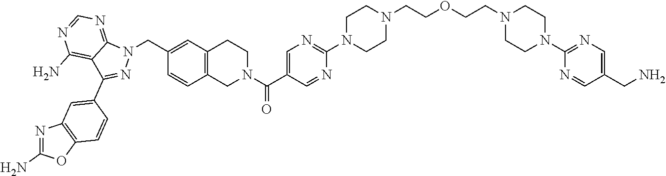 [SMILES: NCc1cnc(N2CCN(CCOCCN3CCN(c4ncc(C(=O)N5CCc6cc(Cn7nc(-c8ccc9oc(N)nc9c8)c8c(N)ncnc87)ccc6C5)cn4)CC3)CC2)nc1] | C₄₄H₅₁N₁₇O₃ | [M + H] = 866.45 | [M + H] = 866.3 |
| Intermediate D1-15 | | | |
| 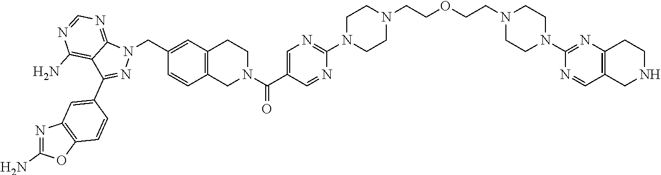 [SMILES: Nc1nc2cc(-c3nn(Cc4ccc5c(c4)CCN(C(=O)c4cnc(N6CCN(CCOCCN7CCN(c8ncc9c(n8)CCNC9)CC7)CC6)nc4)C5)c4ncnc(N)c34)ccc2o1] | C₄₆H₅₃N₁₇O₃ | [M + H] = 892.46 | [M + H] = 892.3 |
| Intermediate D1-16 | | | |
| 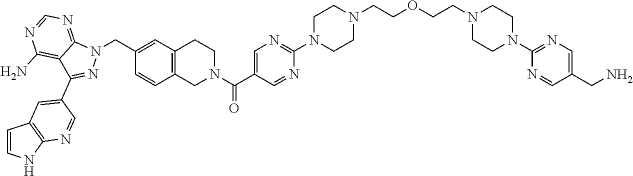 [SMILES: NCc1cnc(N2CCN(CCOCCN3CCN(c4ncc(C(=O)N5CCc6cc(Cn7nc(-c8cnc9[nH]ccc9c8)c8c(N)ncnc87)ccc6C5)cn4)CC3)CC2)nc1] | C₄₄H₅₁N₁₇O₂ | [M + H] = 850.45 | [M + H] = 850.3 |
| Intermediate D1-17 | | | |
| 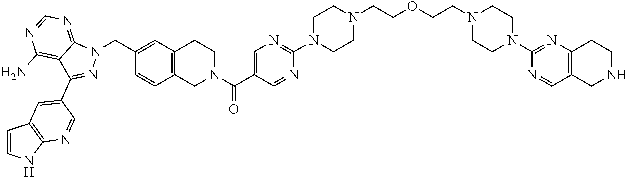 [SMILES: Nc1ncnc2c1c(-c1cnc3[nH]ccc3c1)nn2Cc1ccc2c(c1)CCN(C(=O)c1cnc(N3CCN(CCOCCN4CCN(c5ncc6c(n5)CCNC6)CC4)CC3)nc1)C2] | C₄₆H₅₃N₁₇O₂ | [M + H] = 876.47 | [M + H] = 876.3 |
| Intermediate D1-18 | | | |
| 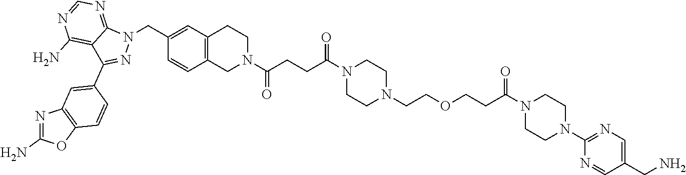 [SMILES: NCc1cnc(N2CCN(C(=O)CCOCCN3CCN(C(=O)CCC(=O)N4CCc5cc(Cn6nc(-c7ccc8oc(N)nc8c7)c7c(N)ncnc76)ccc5C4)CC3)CC2)nc1] | C₄₄H₅₃N₁₅O₅ | [M + H] = 872.45 | [M + H] = 872.3 |
| Intermediate D1-19 | | | |
| 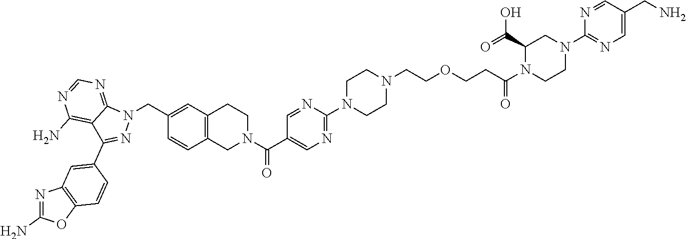 [SMILES: NCc1cnc(N2CCN(C(=O)CCOCCN3CCN(c4ncc(C(=O)N5CCc6cc(Cn7nc(-c8ccc9oc(N)nc9c8)c8c(N)ncnc87)ccc6C5)cn4)CC3)[C@@H](C(=O)O)C2)nc1] | C₄₆H₅₁N₁₇O₆ | [M + H] = 938.61 | [M + H] = 938.3 |
| Intermediate D1-20 | | | |
| 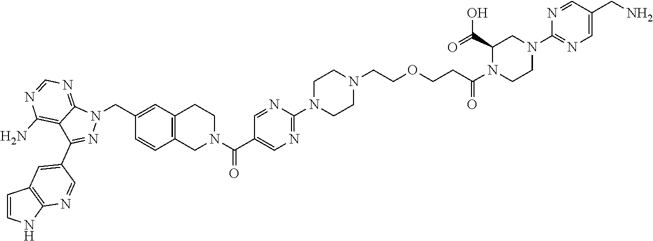 [SMILES: NCc1cnc(N2CCN(C(=O)CCOCCN3CCN(c4ncc(C(=O)N5CCc6cc(Cn7nc(-c8cnc9[nH]ccc9c8)c8c(N)ncnc87)ccc6C5)cn4)CC3)[C@@H](C(=O)O)C2)nc1] | C₄₆H₅₁N₁₇O₅ | [M + H] = 922.44 | [M + H] = 922.3 |
| Intermediate D1-21 | | | |
| 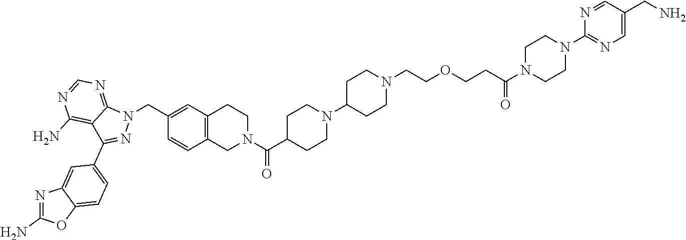 [SMILES: NCc1cnc(N2CCN(C(=O)CCOCCN3CCC(N4CCC(C(=O)N5CCc6cc(Cn7nc(-c8ccc9oc(N)nc9c8)c8c(N)ncnc87)ccc6C5)CC4)CC3)CC2)nc1] | C₄₇H₅₉N₁₅O₄ | [M + H] = 898.50 | [M + H] = 898.4 |
| Intermediate D1-22 | | | |

Following General Procedure 2, but using the appropriate 4-nitrophenyl carbonate containing rapamycin monomer in Table 1 and Intermediates D1 from Tables 19 and 20, the Series 4 bivalent analogs in Table 21 were synthesized:

TABLE 21
Series 4 Bivalent Compounds

| | | Calcu- | Ob- |
|---|---|---|---|
| | Molecular | lated | served |
| Structure | Formula | MW | MW |
| 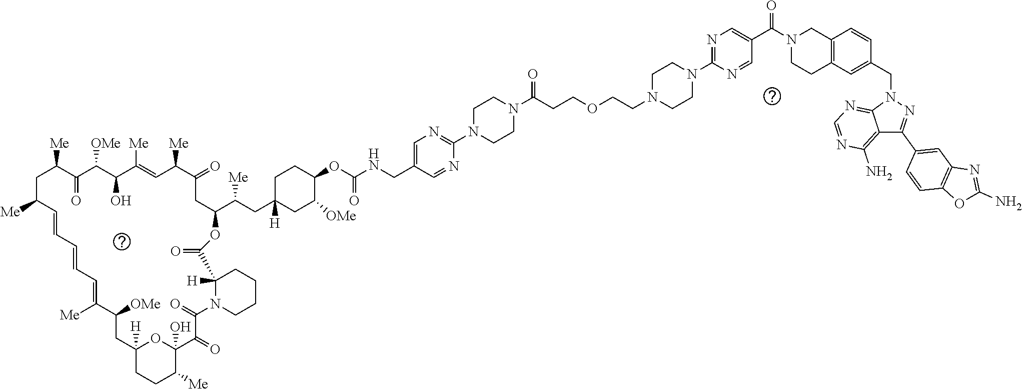 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₈N₁₈O₁₈ | [M + H] = 1833.98 | [M + H] = 1834.1 |
| Example 51 | | | |
| 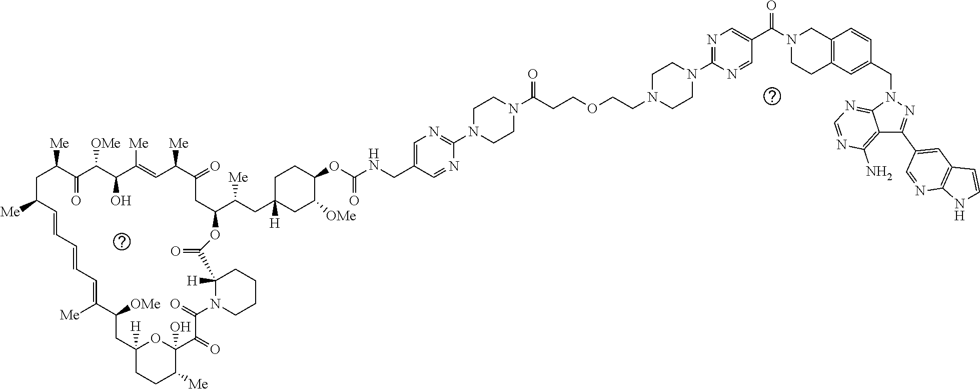 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₈N₁₈O₁₇ | [M + H] = 1817.98 | [M + H] = 1817.9 |
| Example 52 | | | |
| 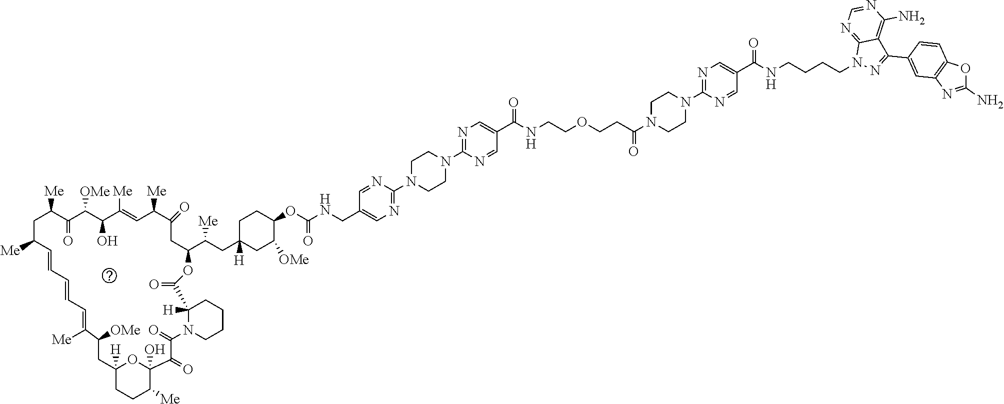 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCC(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₂₉N₂₁O₁₉ | [M + H] = 1880.99 | [M + H] = 1881.0 |
| Example 53 | | | |
| 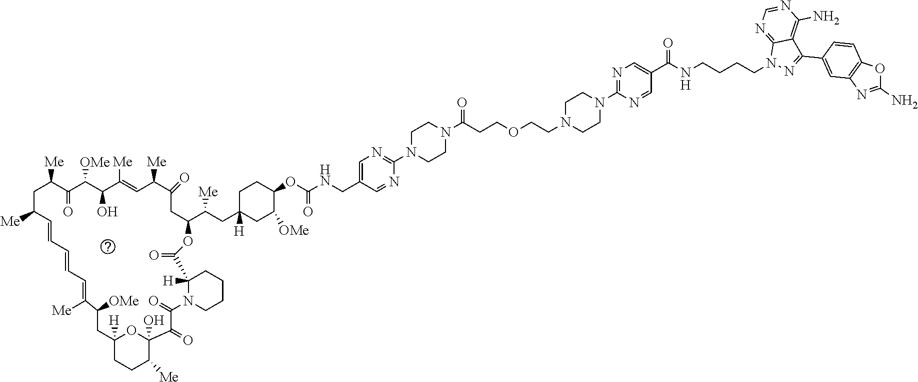 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₂₆N₁₈O₁₈ | [M + H] = 1759.96 | [M + H] = 1760.0 |
| Example 54 | | | |
| 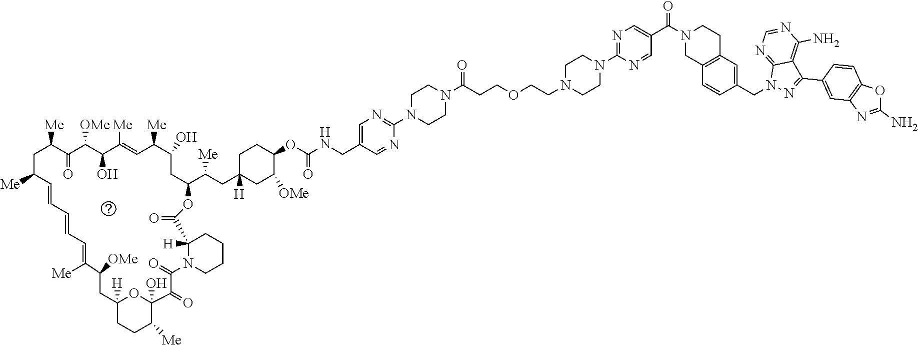 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₇H₁₃₀N₁₈O₁₈ | [M + H] = 1835.99 | [M + H] = 1835.8 |
| Example 55 | | | |
| 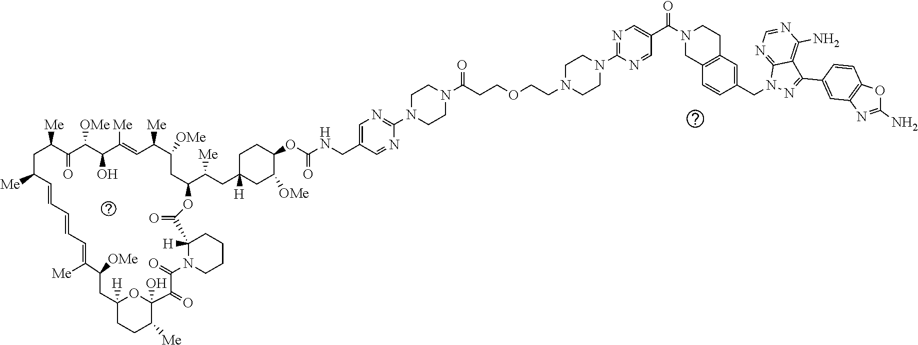 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₈H₁₃₂N₁₈O₁₈ | [M + H] = 1850.01 | [M + H] = 1849.8 |
| Example 56 | | | |
| 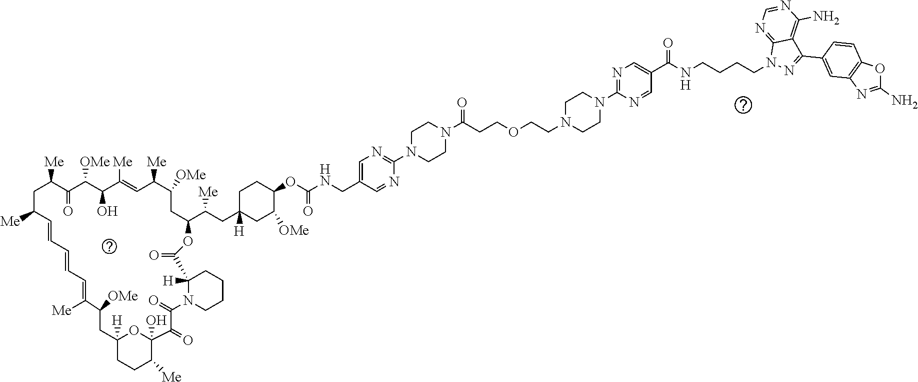 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₂H₁₃₀N₁₈O₁₈ | [M + H] = 1775.99 | [M + H] = 1775.9 |
| Example 57 | | | |
| 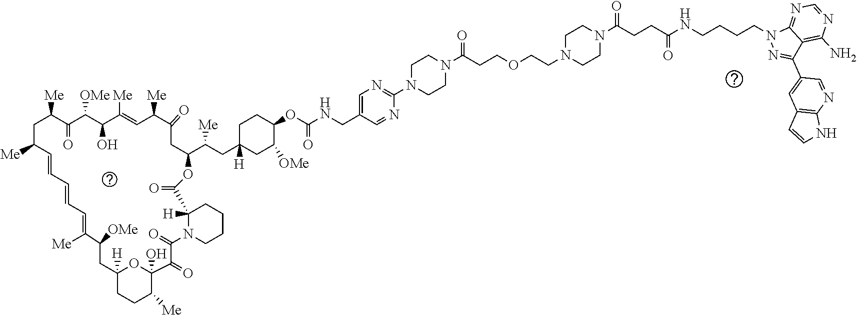 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)NCCCCn6nc(-c7cnc8[nH]ccc8c7)c7c(N)ncnc76)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₂₈N₁₆O₁₈ | [M + H] = 1721.97 | [M + H] = 1721.9 |
| Example 58 | | | |
| 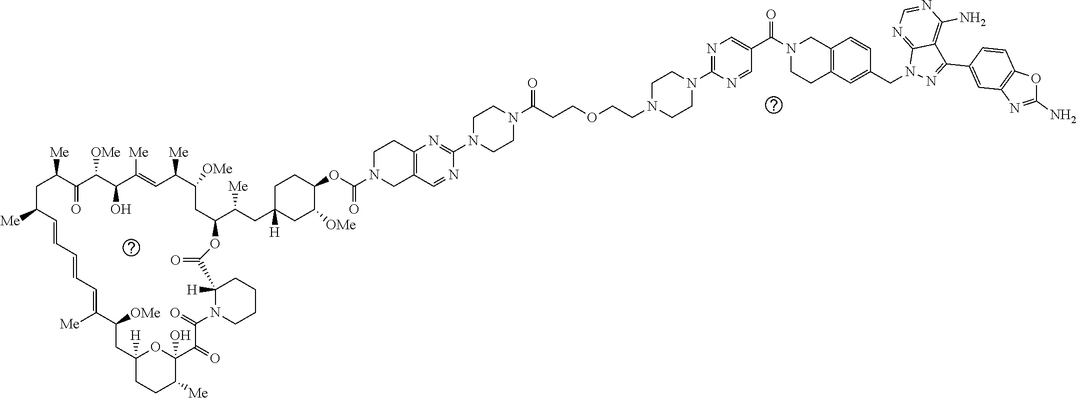 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₄N₁₈O₁₈ | [M + H] = 1876.02 | [M + H] = 1876.1 |
| Example 114 | | | |
| 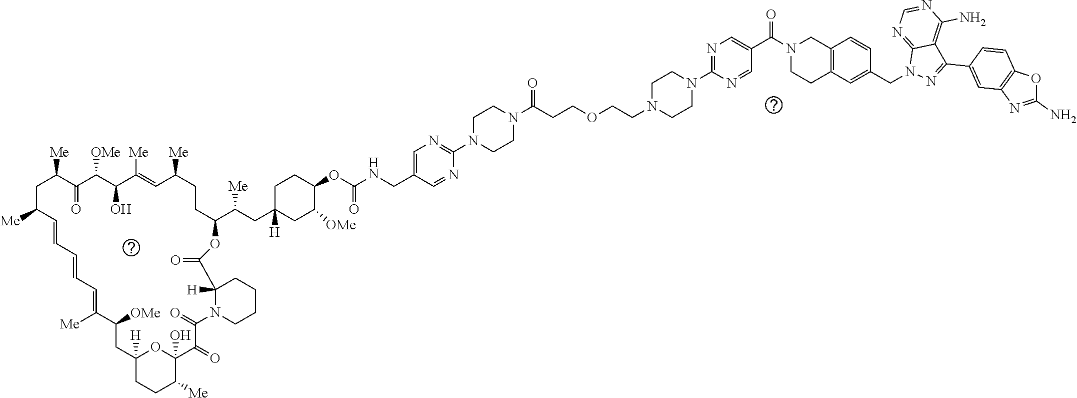 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC[C@H](C)/C=C(\C)[C@@H](O)[C@@H](OC)C(=O)[C@H](C)C[C@H](C)/C=C/C=C/C=C/1C] | C₉₇H₁₃₀N₁₈O₁₇ | [M + H] = 1819.99 | [M + H] = 1820.1 |
| Example 115 | | | |
| 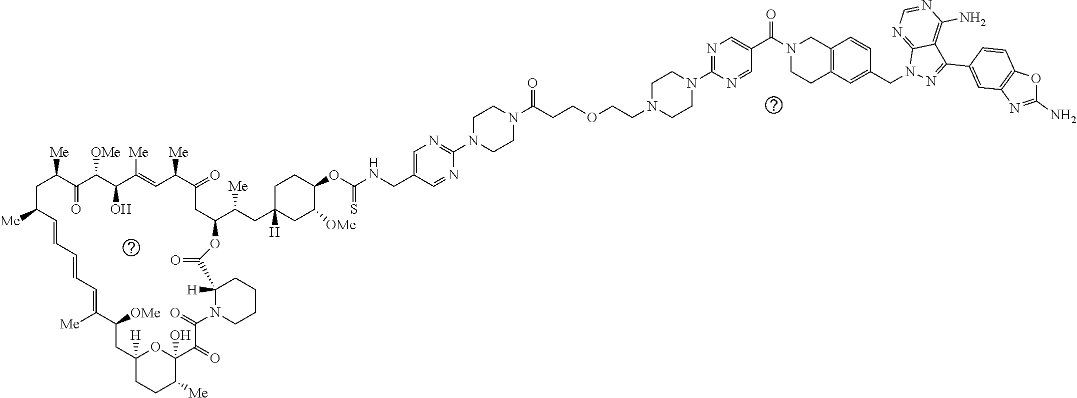 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₈N₁₈O₁₇S | [M + H] = 1849.95 | [M + H] = 1849.9 |
| Example 116 | | | |
| 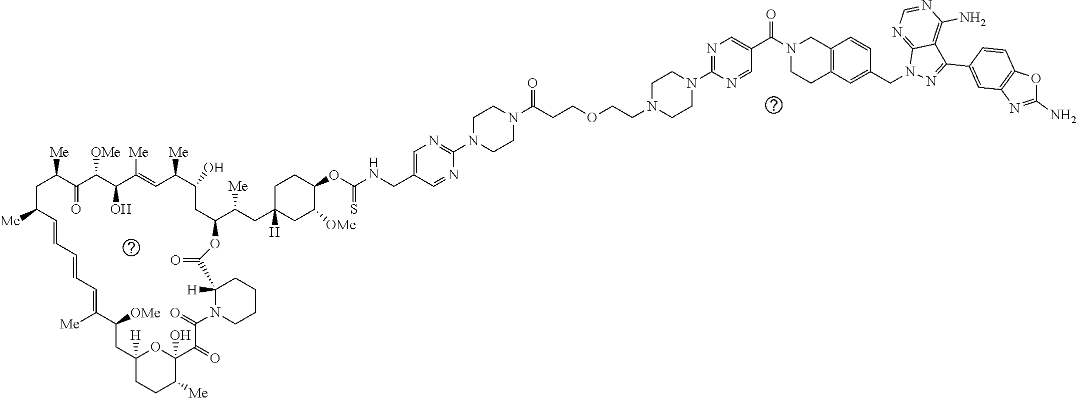 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₇H₁₃₀N₁₈O₁₇S | [M + H] = 1851.97 | [M + H] = 1851.9 |
| Example 117 | | | |
| 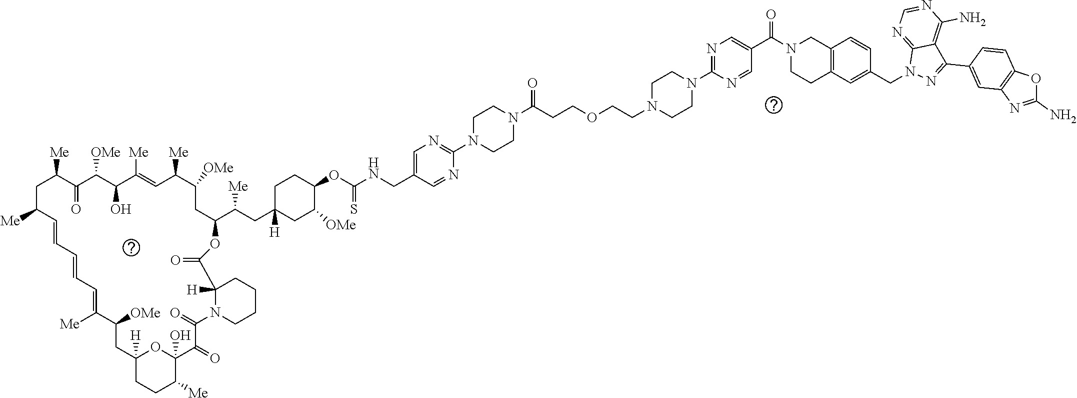 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₈H₁₃₂N₁₈O₁₇S | [M + H] = 1865.98 | [M + H] = 1865.7 |
| Example 118 | | | |
| 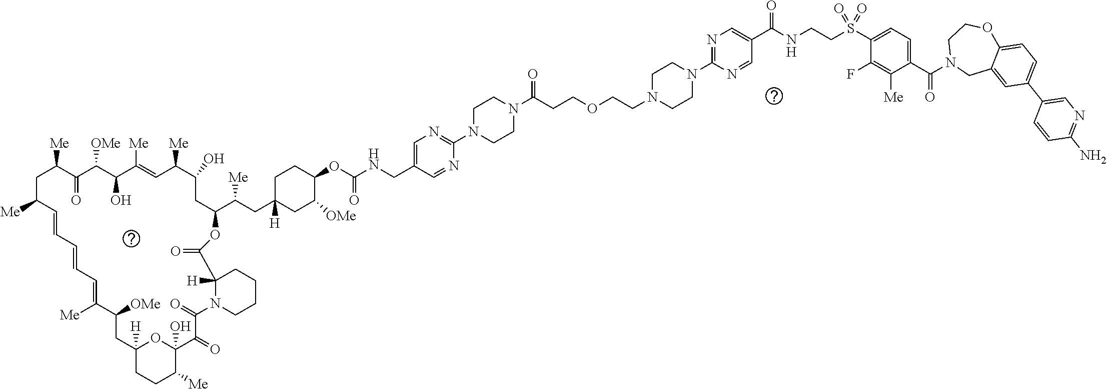 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7F)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₉H₁₃₅FN₁₄O₂₁S | [M + H] = 1907.97 | [M + H] = 1908.0 |
| Example 119 | | | |
| 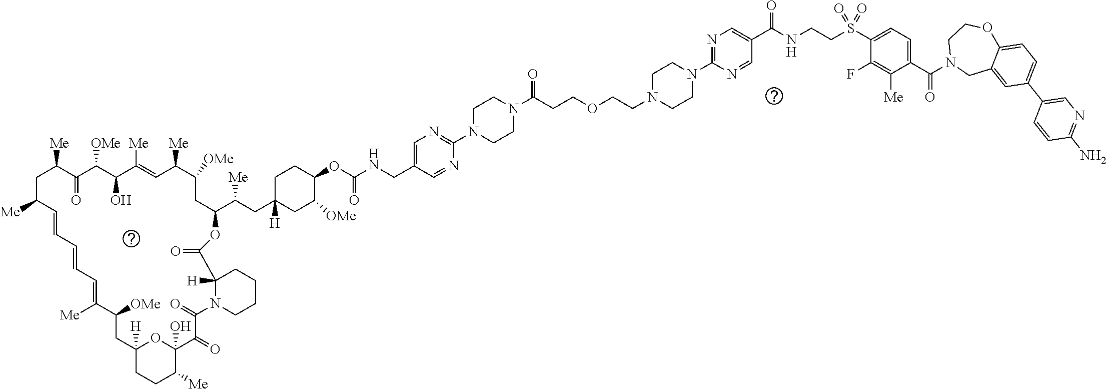 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7F)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₇FN₁₄O₂₁S | [M + H] = 1921.99 | [M + H] = 1922.0 |
| Example 120 | | | |
| 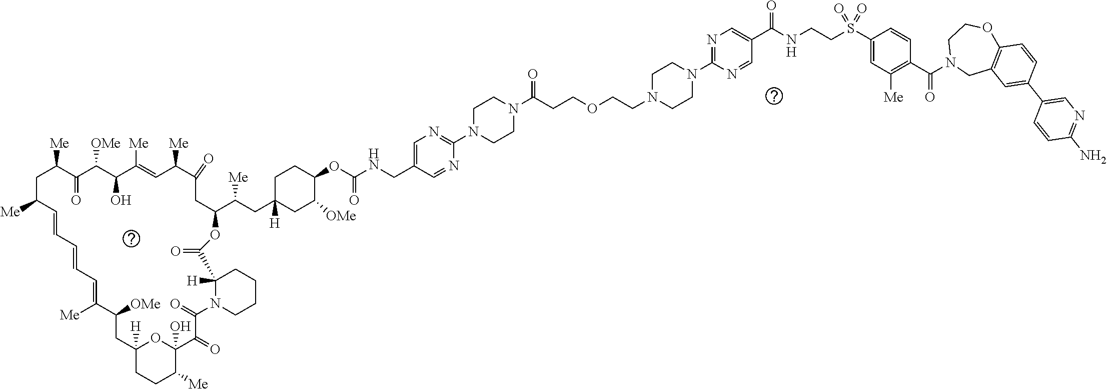 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₄N₁₄O₂₁S | [M + H] = 1887.96 | [M + H] = 1888.0 |
| Example 121 | | | |
| 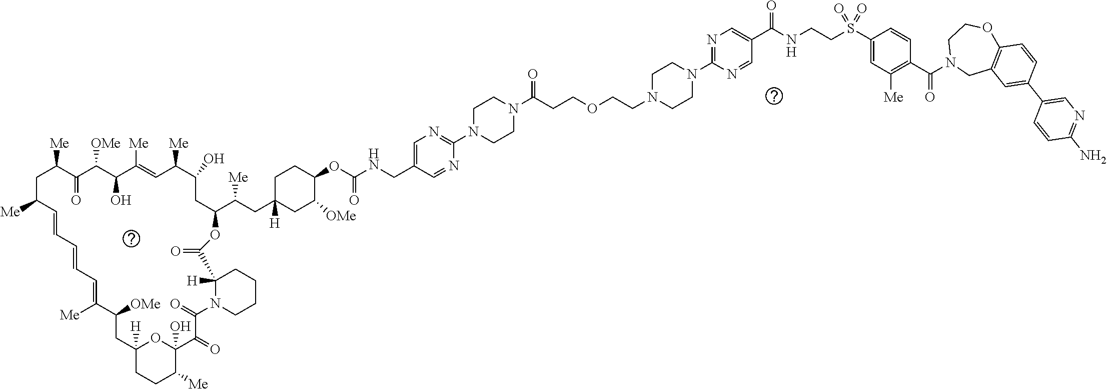 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₉H₁₃₆N₁₄O₂₁S | [M + H] = 1889.98 | [M + H] = 1890.0 |
| Example 122 | | | |
| 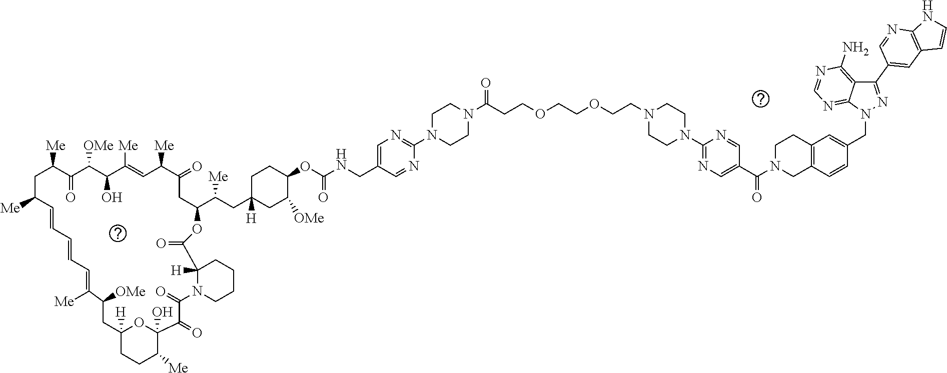 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₂N₁₈O₁₈ | [M + H] = 1862.00 | [M + H] = 1861.9 |
| Example 123 | | | |
| 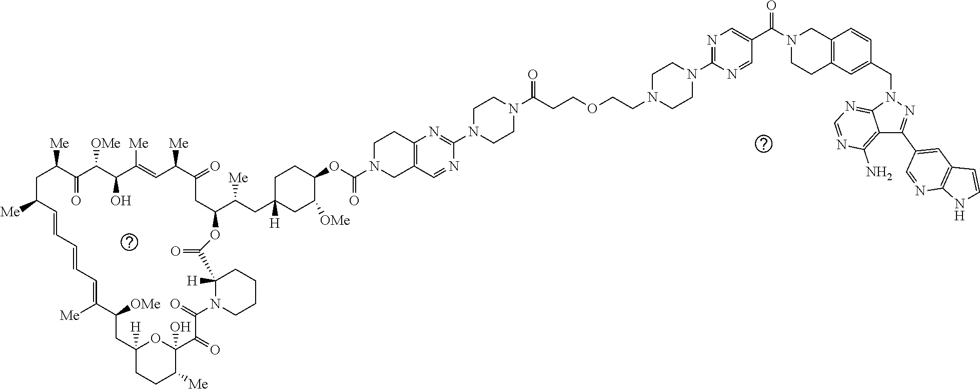 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₀N₁₈O₁₇ | [M + H] = 1843.99 | [M + H] = 1844.1 |
| Example 124 | | | |
| 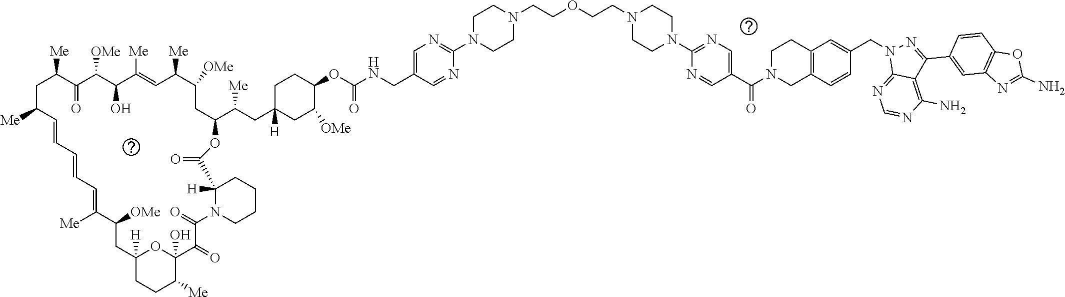 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₇H₁₃₂N₁₈O₁₇ | [M + H] = 1822.01 | [M + H] = 1822.0 |
| Example 125 | | | |
| 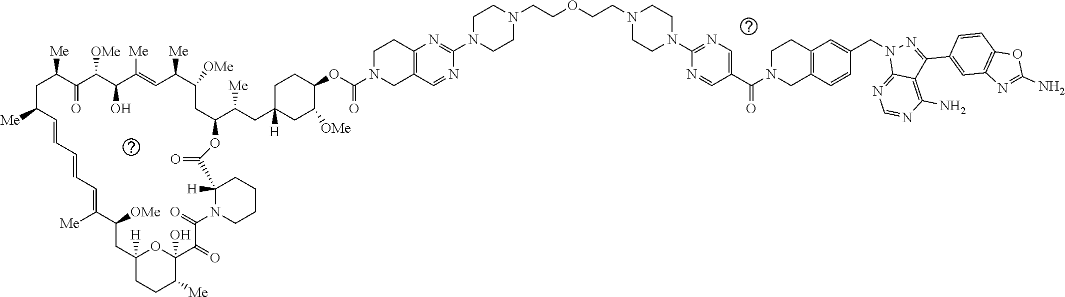 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₉H₁₃₄N₁₈O₁₇ | [M + H] = 1848.03 | [M + H] = 1848.0 |
| Example 126 | | | |
| 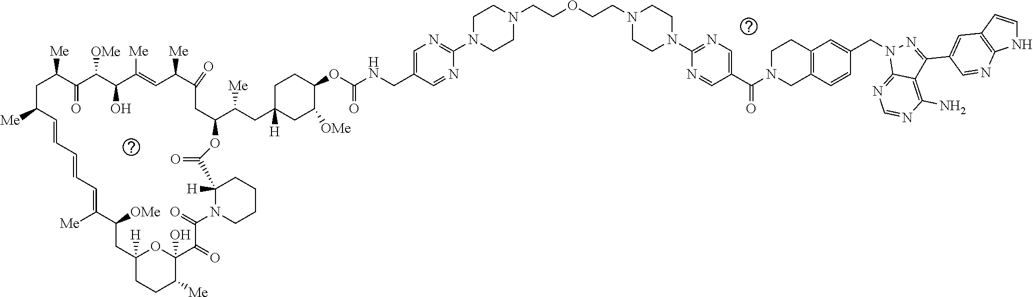 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₂₈N₁₈O₁₆ | [M + H] = 1789.98 | [M + H] = 1789.9 |
| Example 127 | | | |
| 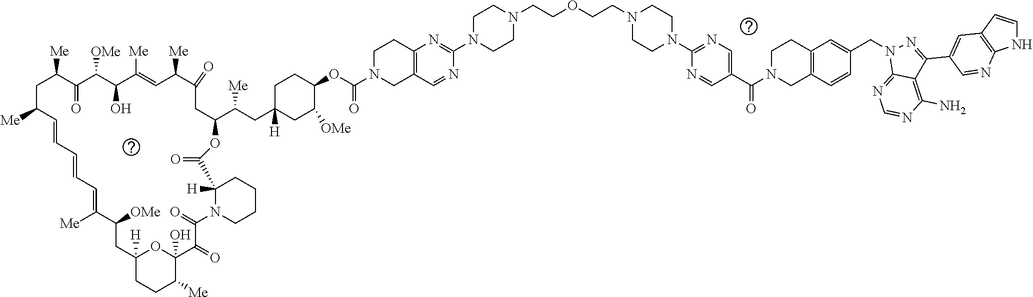 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₃₀N₁₈O₁₆ | [M + H] = 1816.00 | [M + H] = 1815.9 |
| Example 128 | | | |
| 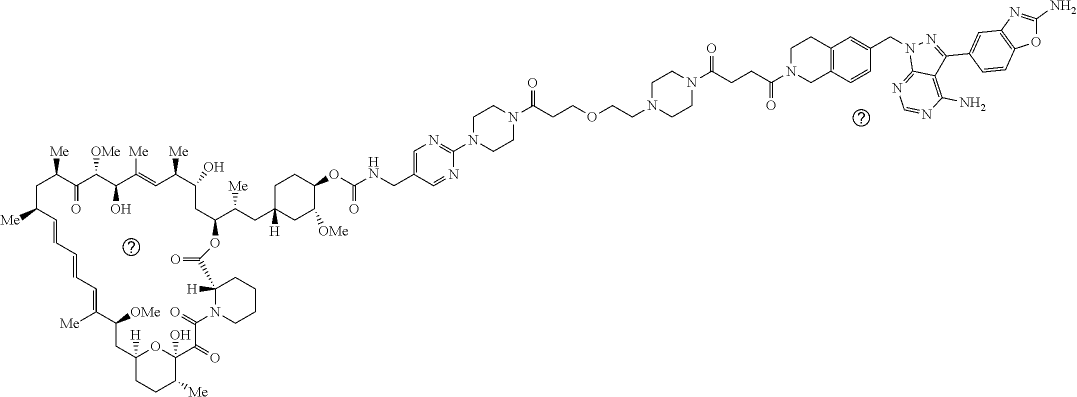 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)N6CCc7cc(Cn8nc(-c9ccc%10oc(N)nc%10c9)c9c(N)ncnc98)ccc7C6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₆H₁₃₂N₁₆O₁₉ | [M + H] = 1813.99 | [M + H] = 1814.0 |
| Example 129 | | | |
| 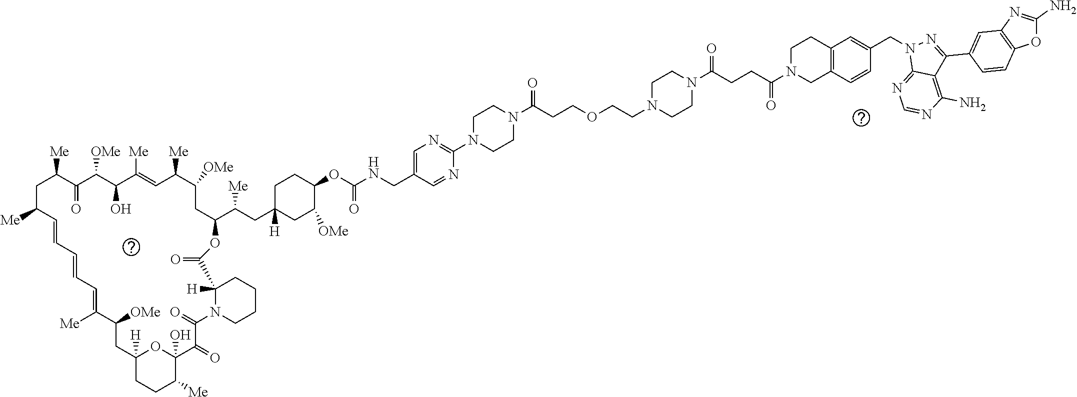 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)N6CCc7cc(Cn8nc(-c9ccc%10oc(N)nc%10c9)c9c(N)ncnc98)ccc7C6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₇H₁₃₄N₁₆O₁₉ | [M + H] = 1828.01 | [M + H] = 1828.0 |
| Example 130 | | | |
| 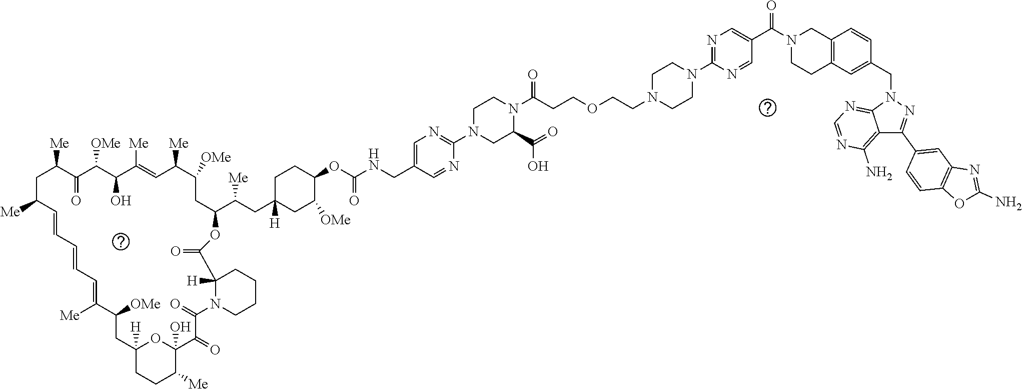 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)[C@@H](C(=O)O)C4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₉H₁₃₂N₁₈O₂₀ | [M + H] = 1893.99 | [M + H] = 1894.0 |
| Example 131 | | | |
| 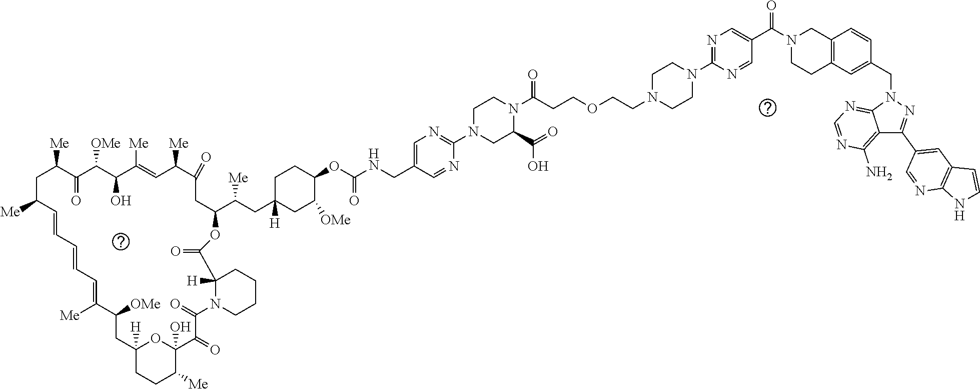 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)[C@@H](C(=O)O)C4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₂₈N₁₈O₁₉ | [M + H] = 1861.97 | [M + H] = 1861.9 |
| Example 132 | | | |
| 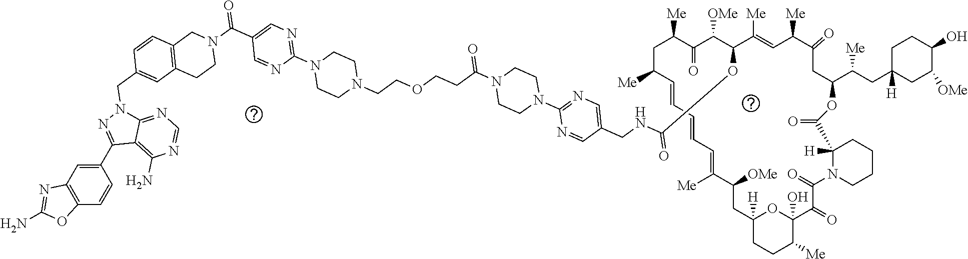 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)NCc2cnc(N3CCN(C(=O)CCOCCN4CCN(c5ncc(C(=O)N6CCc7cc(Cn8nc(-c9ccc%10oc(N)nc%10c9)c9c(N)ncnc98)ccc7C6)cn5)CC4)CC3)nc2)[C@@H](OC)C(=O)[C@H](C)C[C@H](C)/C=C/C=C/C=C/1C] | C₉₇H₁₂₈N₁₈O₁₈ | [M + H] = 1833.97 | [M + H] = 1833.9 |
| Example 133 | | | |
| 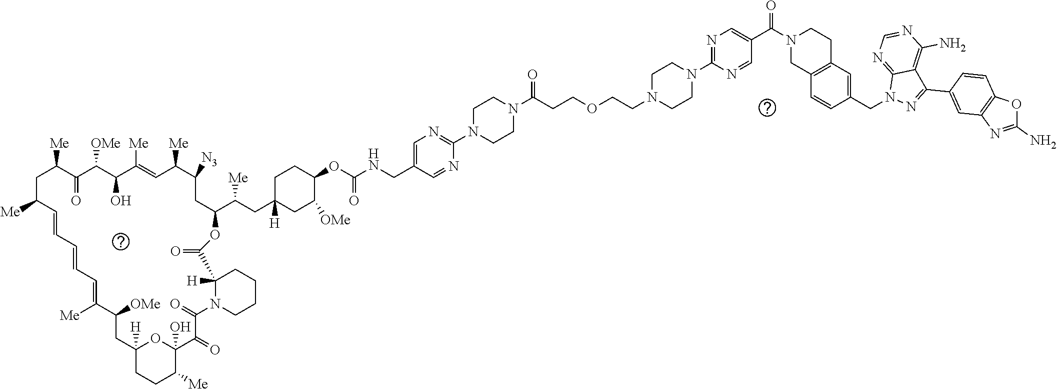 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] | C₉₇H₁₂₉N₂₁O₁₇ | [M + H] = 1861.00 | [M + H] = 1860.8 |
| Example 151 | | | |

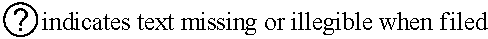

Following General Procedure 1, but using the appropriate Intermediates C1 in Table 16 and amine containing pre-linkers in Table 4, the Intermediates E1 in Table 22 were prepared:

TABLE 22
Additional amines prepared

| | | Calcu- | Ob- |
|---|---|---|---|
| | Molecular | lated | served |
| Structure | Formula | MW | MW |
| 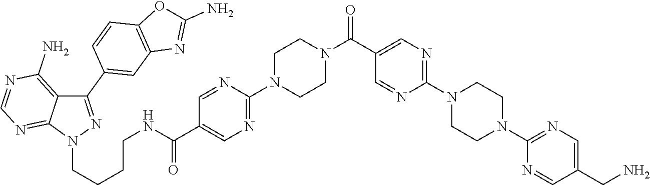 [SMILES: NCc1cnc(N2CCN(c3ncc(C(=O)N4CCN(c5ncc(C(=O)NCCCCn6nc(-c7ccc8oc(N)nc8c7)c7c(N)ncnc76)cn5)CC4)cn3)CC2)nc1] | C₃₉H₄₃N₁₉O₃ | [M + H] = 826.39 | [M + H] = 826.5 |
| Intermediate E1-1 | | | |
| 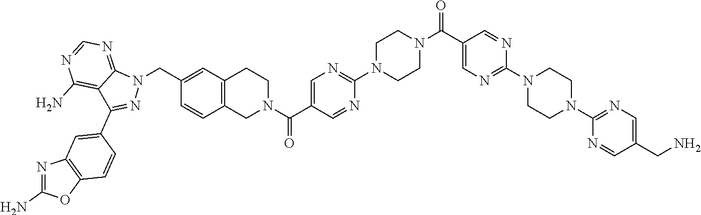 [SMILES: NCc1cnc(N2CCN(c3ncc(C(=O)N4CCN(c5ncc(C(=O)N6CCc7cc(Cn8nc(-c9ccc%10oc(N)nc%10c9)c9c(N)ncnc98)ccc7C6)cn5)CC4)cn3)CC2)nc1] | C₄₅H₄₅N₁₉O₃ | [M + H] = 900.41 | [M + H] = 900.2 |
| Intermediate E1-2 | | | |
| 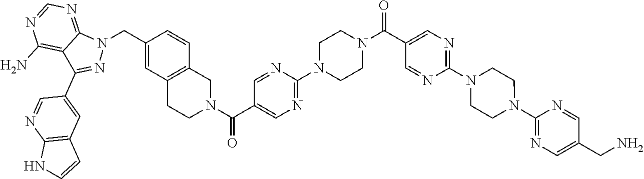 [SMILES: NCc1cnc(N2CCN(c3ncc(C(=O)N4CCN(c5ncc(C(=O)N6CCc7cc(Cn8nc(-c9cnc%10[nH]ccc%10c9)c9c(N)ncnc98)ccc7C6)cn5)CC4)cn3)CC2)nc1] | C₄₅H₄₅N₁₉O₂ | [M + H] = 884.41 | [M + H] = 884.4 |
| Intermediate E1-3 | | | |
| 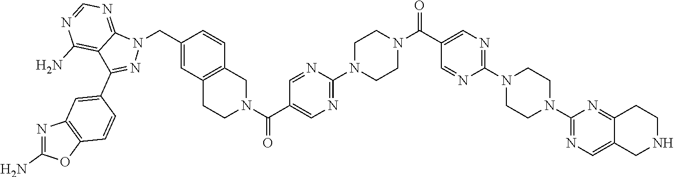 [SMILES: Nc1nc2cc(-c3nn(Cc4ccc5c(c4)CCN(C(=O)c4cnc(N6CCN(C(=O)c7cnc(N8CCN(c9ncc%10c(n9)CCNC%10)CC8)nc7)CC6)nc4)C5)c4ncnc(N)c34)ccc2o1] | C₄₇H₄₇N₁₉O₃ | [M + H] = 926.42 | [M + H] = 926.6 |
| Intermediate E1-4 | | | |
| 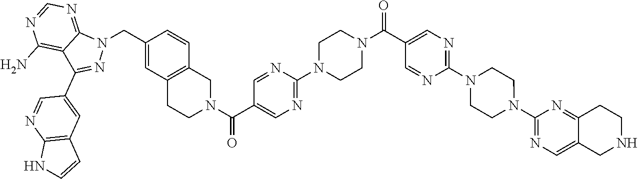 [SMILES: Nc1ncnc2c1c(-c1cnc3[nH]ccc3c1)nn2Cc1ccc2c(c1)CCN(C(=O)c1cnc(N3CCN(C(=O)c4cnc(N5CCN(c6ncc7c(n6)CCNC7)CC5)nc4)CC3)nc1)C2] | C₄₇H₄₇N₁₉O₂ | [M + H] = 910.43 | [M + H] = 910.2 |
| Intermediate E1-5 | | | |
| 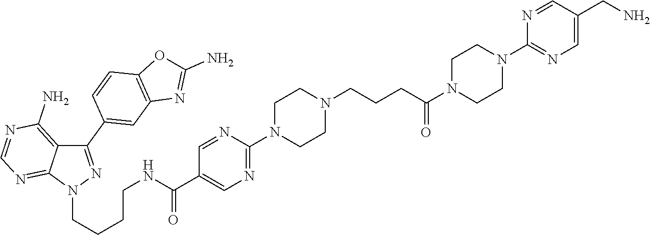 [SMILES: NCc1cnc(N2CCN(C(=O)CCCN3CCN(c4ncc(C(=O)NCCCCn5nc(-c6ccc7oc(N)nc7c6)c6c(N)ncnc65)cn4)CC3)CC2)nc1] | C₃₈H₄₇N₁₇O₃ | [M + H] = 790.41 | [M + H] = 790.4 |
| Intermediate E1-6 | | | |
| 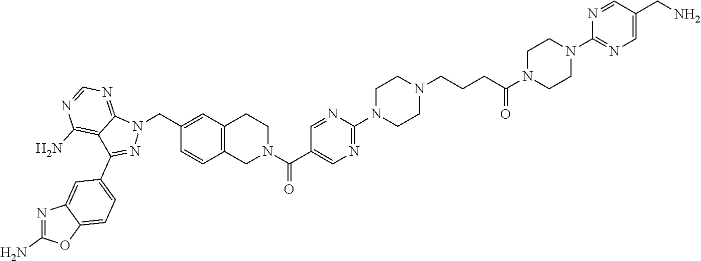 [SMILES: NCc1cnc(N2CCN(C(=O)CCCN3CCN(c4ncc(C(=O)N5CCc6cc(Cn7nc(-c8ccc9oc(N)nc9c8)c8c(N)ncnc87)ccc6C5)cn4)CC3)CC2)nc1] | C₄₄H₄₉N₁₇O₃ | [M + H] = 864.43 | [M + H] = 864.3 |
| Intermediate E1-7 | | | |
| 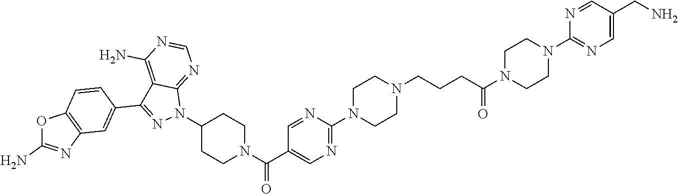 [SMILES: NCc1cnc(N2CCN(C(=O)CCCN3CCN(c4ncc(C(=O)N5CCC(n6nc(-c7ccc8oc(N)nc8c7)c7c(N)ncnc76)CC5)cn4)CC3)CC2)nc1] | C₃₉H₄₇N₁₇O₃ | [M + H] = 802.41 | [M + H] = 802.3 |
| Intermediate E1-8 | | | |

Following General Procedure 2, but using the appropriate 4-nitrophenyl carbonate containing rapamycin monomer in Table 1 and Intermediates E1 from Table 22, the Series 5 bivalent analogs in Table 23 were synthesized:

TABLE 23
Series 5 Bivalent Compounds

| | Molecular | Calculated | Observed |
|---|---|---|---|
| Structure | Formula | MW | MW |
| 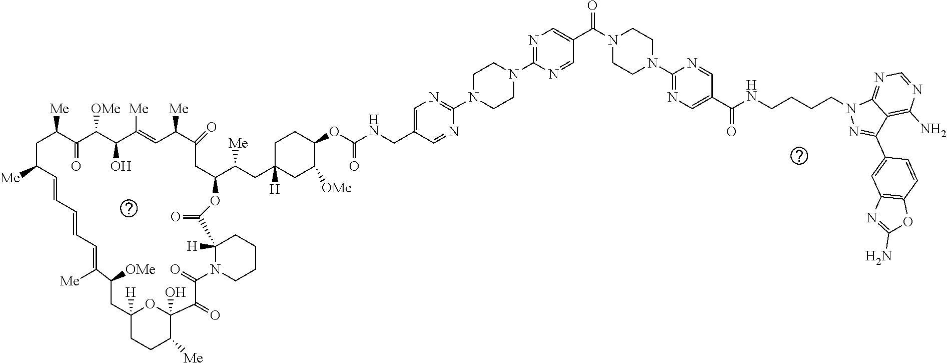 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₂₀N₂₀O₁₇ | [M + H] = 1765.92 | [M + H] = 1765.9 |
| Example 59 | | | |
| 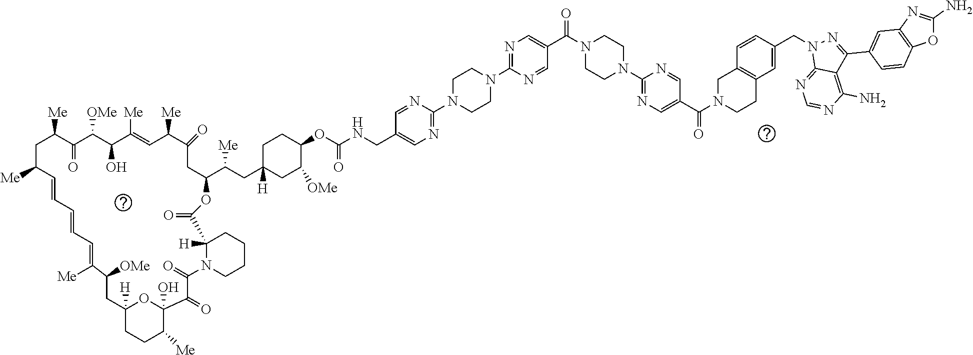 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11ccc%12oc(N)nc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₂N₂₀O₁₇ | [M + H] = 1839.94 | [M + H] = 1840.0 |
| Example 60 | | | |
| 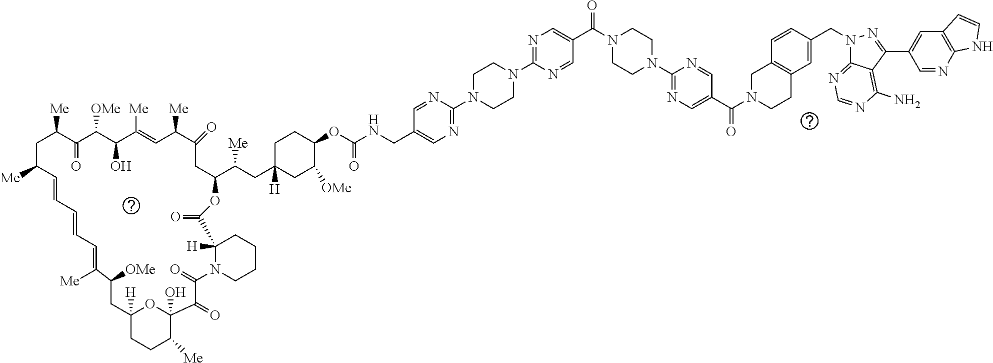 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11cnc%12[nH]ccc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₂N₂₀O₁₆ | [M + H] = 1823.94 | [M + H] = 1823.9 |
| Example 61 | | | |
| 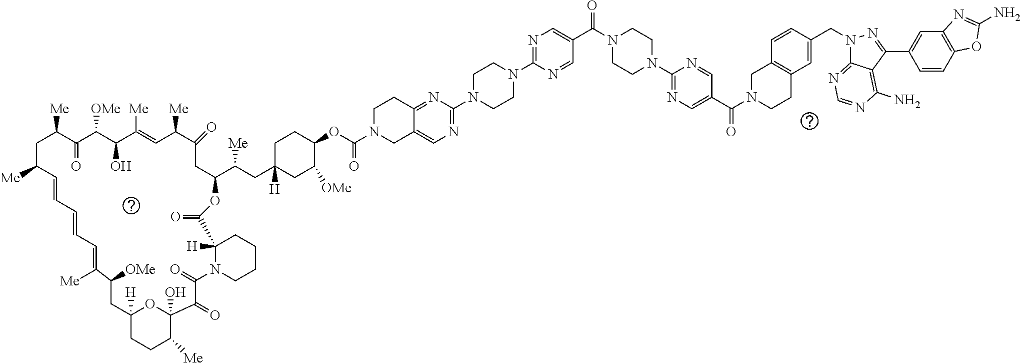 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12ccc%13oc(N)nc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₂₄N₂₀O₁₇ | [M + H] = 1865.95 | [M + H] = 1865.8 |
| Example 62 | | | |
| 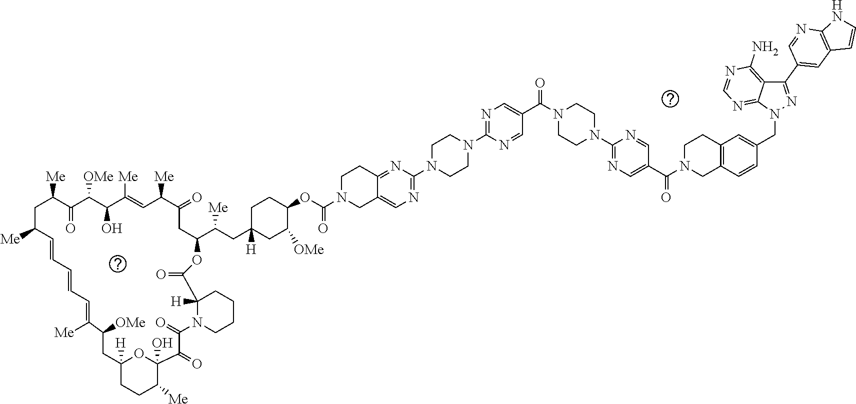 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12cnc%13[nH]ccc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₂₄N₂₀O₁₆ | [M + H] = 1849.96 | [M + H] = 1850.0 |
| Example 134 | | | |

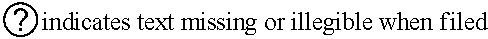

TABLE 24
Additional amines prepared

| | | Calcu- | Ob- |
|---|---|---|---|
| | Molecular | lated | served |
| Structure | Formula | MW | MW |
| 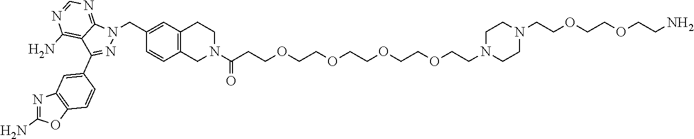 [SMILES: NCCOCCOCCN1CCN(CCOCCOCCOCCOCCC(=O)N2CCc3cc(Cn4nc(-c5ccc6oc(N)nc6c5)c5c(N)ncnc54)ccc3C2)CC1] | C₄₃H₆₁N₁₁O₈ | [M + H] = 860.48 | [M + H] = 860.4 |
| Intermediate F1-1 | | | |
| 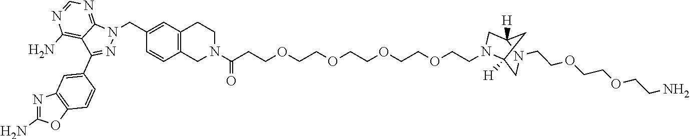 [SMILES: NCCOCCOCCN1C[C@@H]2C[C@H]1CN2CCOCCOCCOCCOCCC(=O)N1CCc2cc(Cn3nc(-c4ccc5oc(N)nc5c4)c4c(N)ncnc43)ccc2C1] | C₄₄H₆₁N₁₁O₈ | [M + H] = 872.48 | [M + H] = 872.2 |
| Intermediate F1-2 | | | |

Following General Procedure 2, but using the appropriate 4-nitrophenyl carbonate containing rapamycin monomer in Table 1 and Intermediates F1 from Table 24, the Series 6 bivalent analogs in Table 25 were synthesized:

TABLE 25
Series 6 Bivalent Compounds

| | Molecular | Calculated | Observed |
|---|---|---|---|
| Structure | Formula | MW | MW |
| 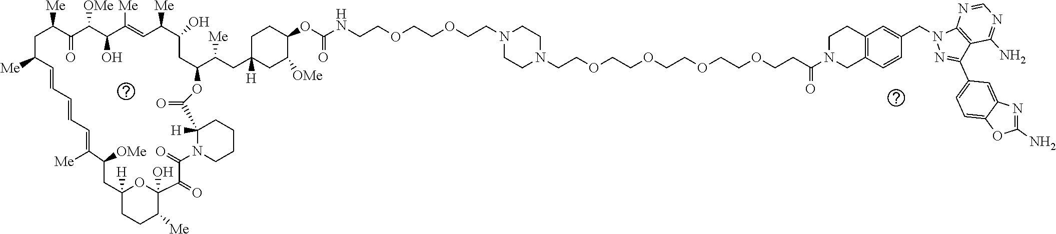 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3CCN(CCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₀N₁₂O₂₂ | [M + H] = 1802.03 | [M + H] = 1802.5 |
| Example 135 | | | |
| 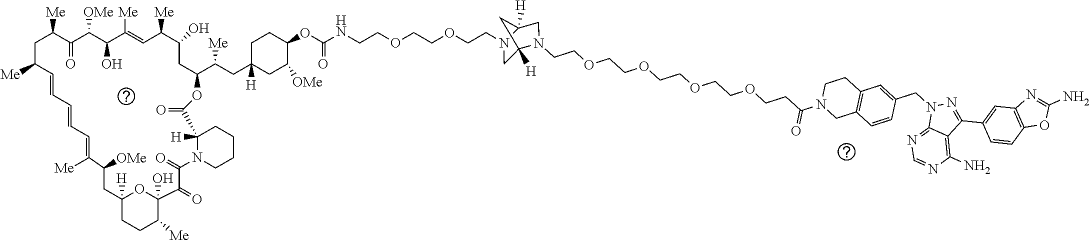 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3C[C@@H]4C[C@H]3CN4CCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₆H₁₄₀N₁₂O₂₂ | [M + H] = 1814.03 | [M + H] = 1813.9 |
| Example 152 | | | |

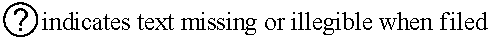

Following General Procedure 1, but using the appropriate Intermediates A1 in Table 5 and amine containing pre-linkers in Table 4, the Intermediates G1 in Table 26 were prepared:

TABLE 26
Additional amines prepared

| | Molecular | Calculated | Observed |
|---|---|---|---|
| Structure | Formula | MW | MW |
| 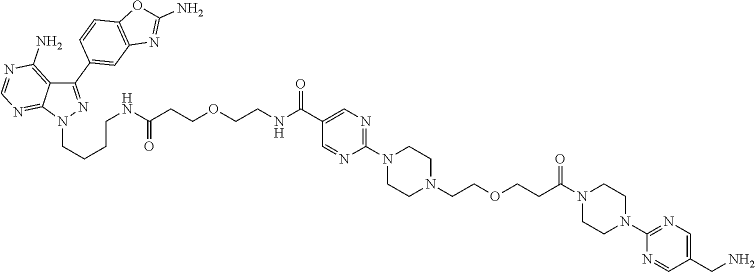 [SMILES: NCc1cnc(N2CCN(C(=O)CCOCCN3CCN(c4ncc(C(=O)NCCOCCC(=O)NCCCCn5nc(-c6ccc7oc(N)nc7c6)c6c(N)ncnc65)cn4)CC3)CC2)nc1] | C₄₄H₅₈N₁₈O₆ | [M + H] = 935.49 | [M + H] = 935.5 |
| Intermediate G1-1 | | | |
| 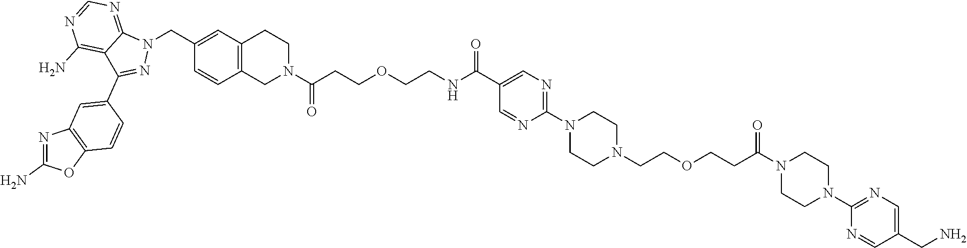 [SMILES: NCc1cnc(N2CCN(C(=O)CCOCCN3CCN(c4ncc(C(=O)NCCOCCC(=O)N5CCc6cc(Cn7nc(-c8ccc9oc(N)nc9c8)c8c(N)ncnc87)ccc6C5)cn4)CC3)CC2)nc1] | C₅₀H₆₀N₁₈O₆ | [M + H] = 1009.50 | [M + H] = 1009.5 |
| Intermediate G1-2 | | | |
| 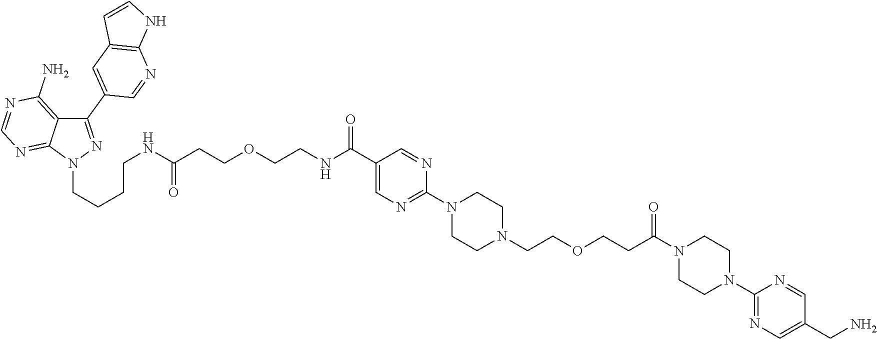 [SMILES: NCc1cnc(N2CCN(C(=O)CCOCCN3CCN(c4ncc(C(=O)NCCOCCC(=O)NCCCCn5nc(-c6cnc7[nH]ccc7c6)c6c(N)ncnc65)cn4)CC3)CC2)nc1] | C₄₄H₅₈N₁₈O₅ | [M + H] = 919.49 | [M + H] = 919.5 |
| Intermediate G1-3 | | | |

Following General Procedure 6, but using the appropriate Intermediates B3 in Table 10 and amine containing pre-linkers in Table 4, the Intermediates G1 in Table 27 were prepared:

TABLE 27
Additional amines prepared

| | | Calcu- | Ob- |
|---|---|---|---|
| | Molecular | lated | served |
| Structure | Formula | MW | MW |
| 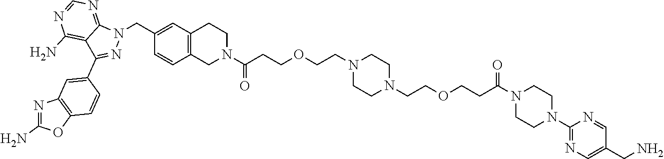 [SMILES: NCc1cnc(N2CCN(C(=O)CCOCCN3CCN(CCOCCC(=O)N4CCc5cc(Cn6nc(-c7ccc8oc(N)nc8c7)c7c(N)ncnc76)ccc5C4)CC3)CC2)nc1] | C₄₅H₅₇N₁₅O₅ | [M + H] = 888.48 | [M + H] = 888.4 |
| Intermediate G1-4 | | | |

Following General Procedure 2, but using the appropriate 4-nitrophenyl carbonate containing rapamycin monomer in Table 1 and Intermediates G1 from Tables 26 and 27, the Series 7 bivalent analogs in Table 28 were synthesized:

TABLE 28
Series 7 Bivalent Compounds

| | Molecular | Calculated | Observed |
|---|---|---|---|
| Structure | Formula | MW | MW |
| 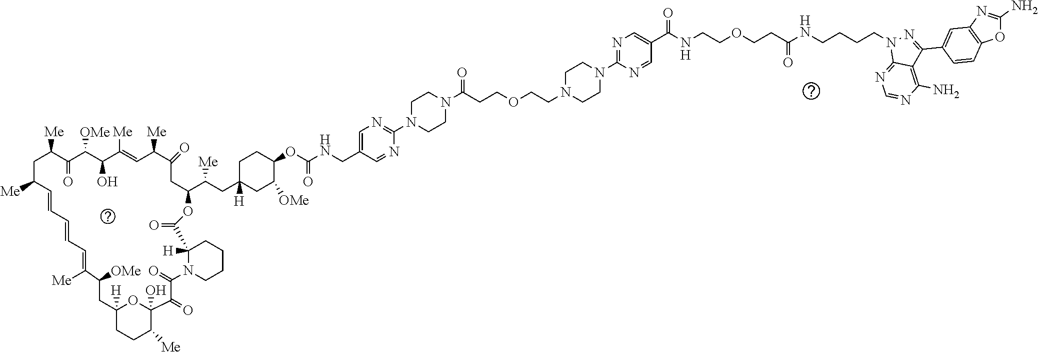 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₅N₁₉O₂₀ | [M + H] = 1875.02 | [M + H] = 1874.9 |
| Example 63 | | | |
| 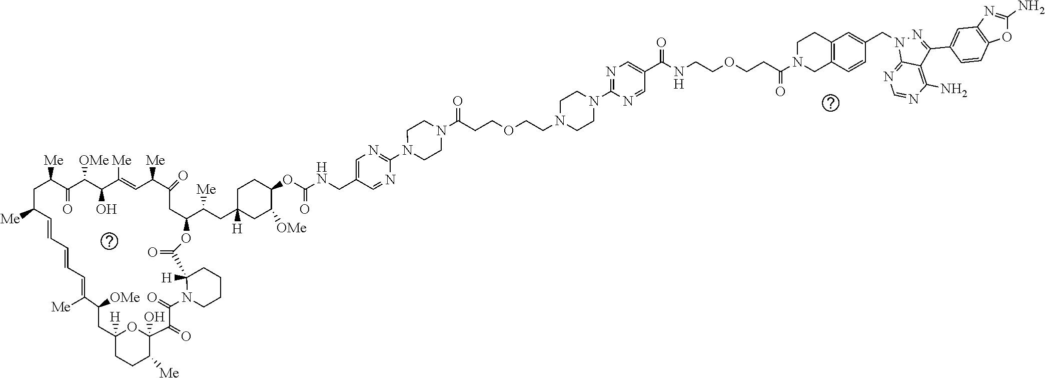 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₂H₁₃₇N₁₉O₂₀ | [M + H] = 1949.04 | [M + H] = 1949.0 |
| Example 64 | | | |
| 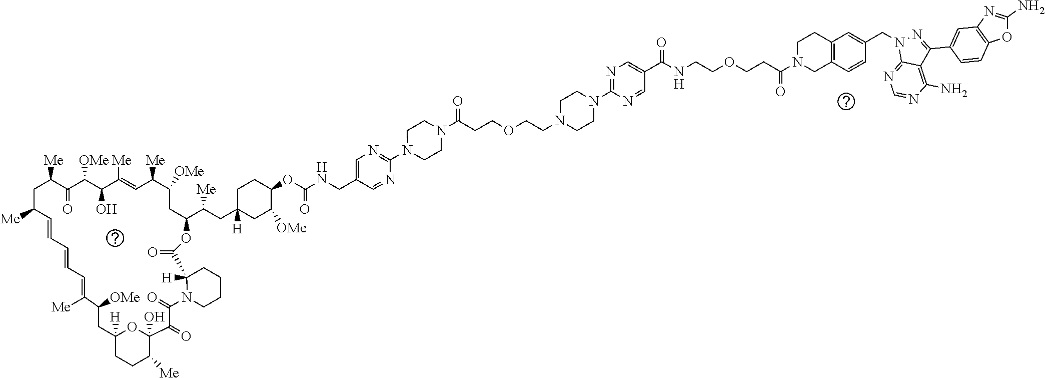 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₃H₁₄₁N₁₉O₂₀ | [M + H] = 1965.07 | [M + H] = 1965.0 |
| Example 65 | | | |
| 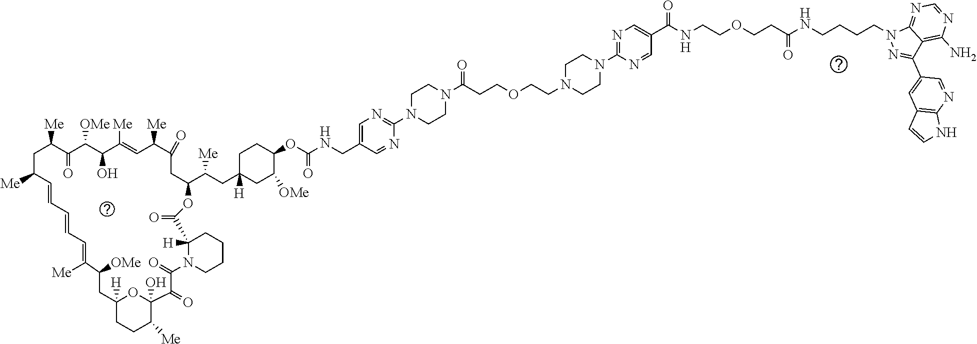 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₅N₁₉O₁₉ | [M + H] = 1859.03 | [M + H] = 1859.0 |
| Example 66 | | | |

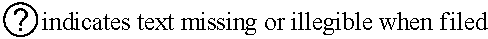

Following General Procedure 1, but using the appropriate Intermediates D1 in Tables 19 and 20 and PEG carboxylic acids, the Intermediates H1 in Table 29 were prepared:

TABLE 29
Additional amines prepared

| | Molecular | Calculated | Observed |
|---|---|---|---|
| Structure | Formula | MW | MW |
| 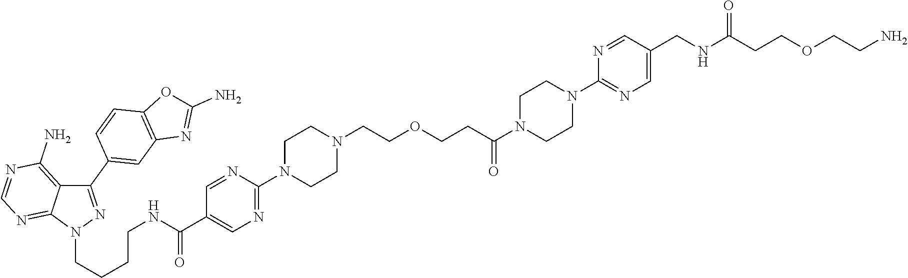 [SMILES: NCCOCCC(=O)NCc1cnc(N2CCN(C(=O)CCOCCN3CCN(c4ncc(C(=O)NCCCCn5nc(-c6ccc7oc(N)nc7c6)c6c(N)ncnc65)cn4)CC3)CC2)nc1] | C₄₄H₅₈N₁₈O₆ | [M + H] = 935.49 | [M + H] = 935.5 |
| Intermediate H1-1 | | | |
| 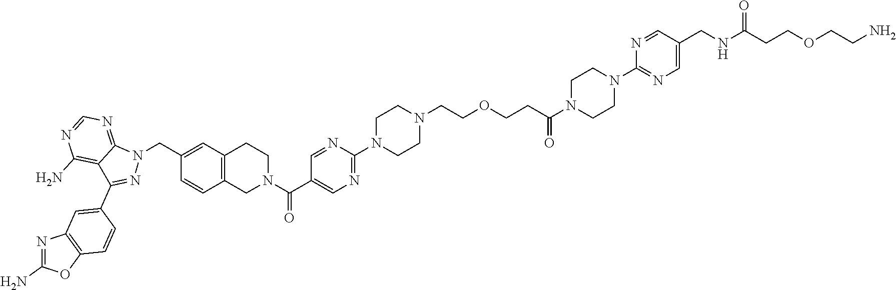 [SMILES: NCCOCCC(=O)NCc1cnc(N2CCN(C(=O)CCOCCN3CCN(c4ncc(C(=O)N5CCc6cc(Cn7nc(-c8ccc9oc(N)nc9c8)c8c(N)ncnc87)ccc6C5)cn4)CC3)CC2)nc1] | C₅₀H₆₀N₁₈O₆ | [M + 2H]/2 = 505.23 | [M + 2H]/2 = 505.4 |
| Intermediate H1-2 | | | |
| 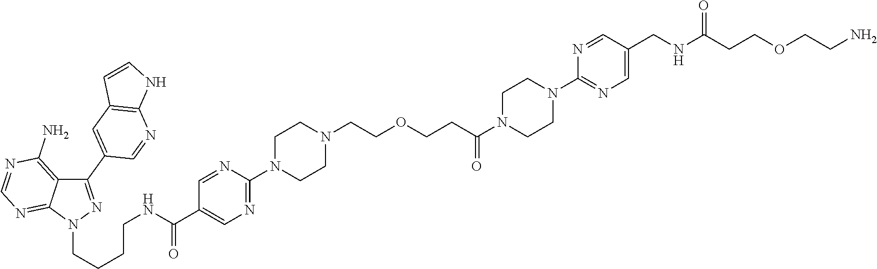 [SMILES: NCCOCCC(=O)NCc1cnc(N2CCN(C(=O)CCOCCN3CCN(c4ncc(C(=O)NCCCCn5nc(-c6cnc7[nH]ccc7c6)c6c(N)ncnc65)cn4)CC3)CC2)nc1] | C₄₄H₅₈N₁₈O₅ | [M + 2H]/2 = 460.25 | [M + 2H]/2 = 460.3 |
| Intermediate H1-3 | | | |

Following General Procedure 2, but using the appropriate 4-nitrophenyl carbonate containing rapamycin monomer in Table 1 and Intermediates H1 from Table 29, the Series 8 bivalent analogs in Table 30 were synthesized:

TABLE 30
Series 8 Bivalent Compounds:

| | | Calcu- | Ob- |
|---|---|---|---|
| | Molecular | lated | served |
| Structure | Formula | MW | MW |
| 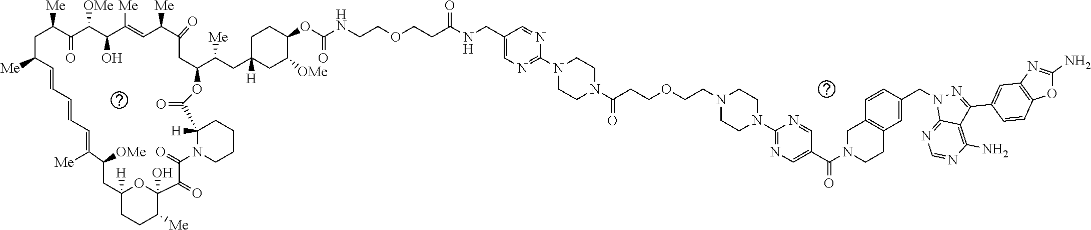 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₂H₁₃₇N₁₉O₂₀ | [M + H] = 1949.04 | [M + H] = 1949.0 |
| Example 67 | | | |
| 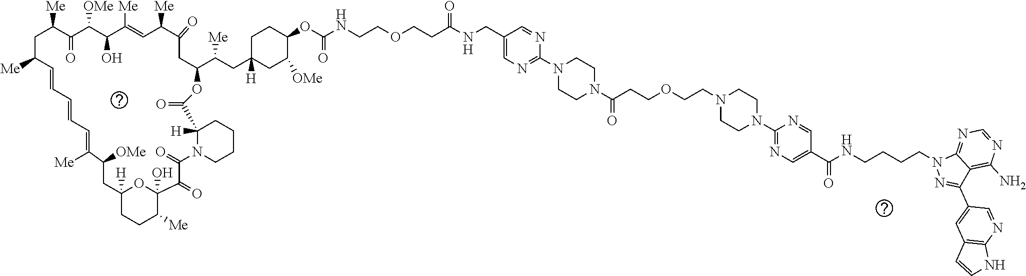 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₅N₁₉O₁₉ | [M + H] = 1859.03 | [M + H] = 1859.0 |
| Example 68 | | | |
| 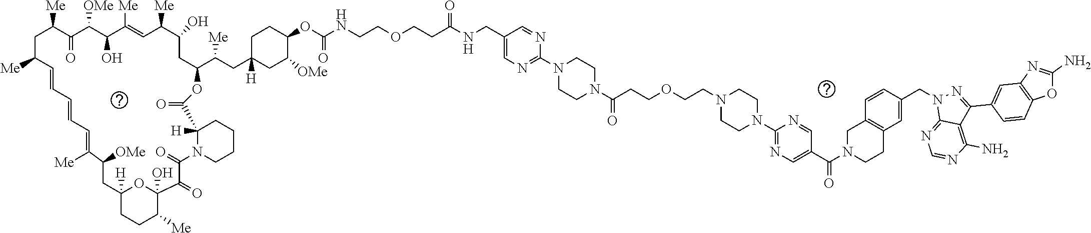 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₁₀₂H₁₃₉N₁₉O₂₀ | [M + H] = 1951.05 | [M + H] = 1951.1 |
| Example 69 | | | |
| 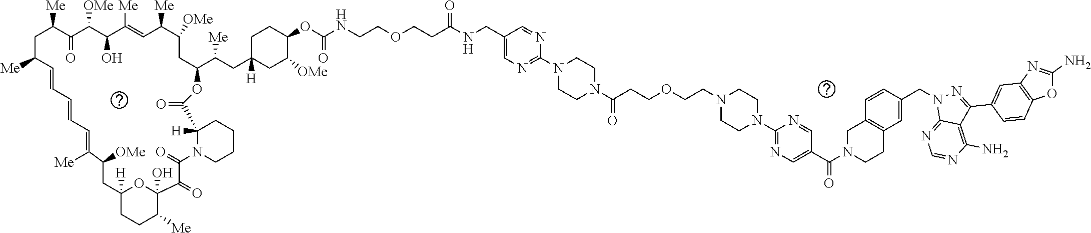 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₃H₁₄₁N₁₉O₂₀ | [M + H] = 1965.07 | [M + H] = 1965.1 |
| Example 70 | | | |

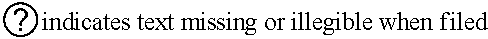

### Biological Examples

### Cell Based AlphaLISA Assays for Determining IC50 For Inhibition of P-Akt (S473), P-4E-BP1 (T37/46), and P-P70S6K (T389) in MDA-MB-468 Cells

mTOR Kinase Cellular Assay

To measure functional activity of mTORC1 and mTORC2 in cells the phosphorylation of 4EBP1 (Thr37/46) and P70S6K (Thr389), and AKT1/2/3 (Ser473) was monitored using AlphaLisa SureFire Ultra Kits (Perkin Elmer). MDA-MB-468 cells (ATCC® HTB-132) were cultured in 96-well tissue culture plates and treated with compounds in the disclosure at concentrations varying from 0.017-1,000 nM for two to four hours at 37° C. Incubations were terminated by removal of the assay buffer and addition of lysis buffer provided with the assay kit. Samples were processed according to the manufacturer's instructions. The Alpha signal from the respective phosphoproteins was measured in duplicate using a microplate reader (Envision, Perkin-Elmer or Spectramax M5, Molecular Devices). Inhibitor concentration response curves were analyzed using normalized IC₅₀ regression curve fitting with control based normalization.

As an example, measured IC₅₀ values for selected compounds are reported below:

| | IC₅₀ for Inhibition of mTORC1 and mTORC2 Substarte | | |
|---|---|---|---|
| | Phosphorylation (nM) | | |
| | p-P70S6K- | p-4E-BP1- | p-AKT1/2/3- |
| Compound | (T389) | (T37/46) | (S473) |
| MLN-128 | 1.4 | 16 | 3.7 |
| Rapamycin | 0.2 | >1,000 | >1,000 |

As an example, measured IC₅₀ values for selected compounds are reported below:

| | pIC₅₀ for Inhibition of mTORC1 and mTORC2 Substarte | | |
|---|---|---|---|
| | Phosphorylation | | |
| | p-P70S6K- | p-4E-BP1- | p-AKT1/2/3- |
| Example | (T389) | (T37/46) | (S473) |
| 1 | +++ | +++ | ++ |
| 2 | +++ | +++ | ++ |
| 3 | +++ | +++ | ++ |
| 4 | +++ | +++ | ++ |
| 5 | +++ | +++ | + |
| 6 | +++ | +++ | ++ |
| 7 | +++ | +++ | ++ |
| 8 | +++ | +++ | ++ |
| 9 | +++ | +++ | ++ |
| 10 | +++ | − | − |
| 11 | +++ | +++ | ++ |
| 12 | +++ | +++ | + |
| 13 | +++ | +++ | ++ |
| 14 | ++ | +++ | ++ |
| 15 | +++ | +++ | ++ |
| 16 | +++ | +++ | +++ |
| 17 | +++ | +++ | ++ |
| 18 | +++ | ++ | + |
| 19 | +++ | +++ | +++ |
| 20 | +++ | +++ | +++ |
| 21 | +++ | +++ | + |
| 27 | +++ | +++ | ++ |
| 28 | +++ | +++ | − |
| 29 | +++ | +++ | − |
| 30 | +++ | +++ | ++ |
| 31 | +++ | +++ | ++ |
| 32 | +++ | +++ | ++ |
| 33 | +++ | +++ | − |
| 34 | +++ | − | − |
| 35 | +++ | +++ | ++ |
| 36 | +++ | +++ | − |
| 37 | +++ | +++ | − |
| 38 | +++ | − | − |
| 39 | +++ | +++ | +++ |
| 40 | +++ | +++ | ++ |
| 41 | +++ | +++ | − |
| 42 | +++ | +++ | ++ |
| 43 | +++ | +++ | ++ |
| 46 | +++ | +++ | ++ |
| 47 | +++ | +++ | ++ |
| 48 | +++ | +++ | ++ |
| 49 | +++ | +++ | ++ |
| 50 | +++ | +++ | + |
| 51 | +++ | +++ | +++ |
| 52 | +++ | +++ | − |
| 53 | +++ | +++ | ++ |
| 54 | +++ | +++ | +++ |
| 55 | +++ | +++ | ++ |
| 56 | +++ | +++ | − |
| 59 | +++ | +++ | +++ |
| 60 | +++ | ++ | + |
| 61 | +++ | − | − |
| 62 | ++ | − | − |
| 63 | +++ | +++ | +++ |
| 64 | +++ | +++ | ++ |
| 65 | +++ | +++ | + |
| 66 | + | + | ++ |
| 67 | + | + | + |
| 68 | +++ | +++ | − |
| 69 | +++ | +++ | ++ |
| 70 | +++ | +++ | + |
| 71 | +++ | +++ | ++ |
| 72 | +++ | +++ | +++ |
| 73 | +++ | +++ | + |
| 74 | +++ | +++ | + |
| 75 | +++ | +++ | ++ |
| 76 | +++ | +++ | ++ |
| 77 | +++ | ++ | + |
| 78 | +++ | +++ | +++ |
| 79 | +++ | +++ | + |
| 80 | +++ | +++ | +++ |
| 81 | +++ | ++ | − |
| 82 | +++ | +++ | − |
| 83 | +++ | +++ | ++ |
| 84 | +++ | +++ | ++ |
| 85 | +++ | +++ | ++ |
| 86 | +++ | +++ | ++ |
| 87 | +++ | +++ | ++ |
| 88 | +++ | +++ | ++ |
| 89 | +++ | +++ | − |
| 90 | +++ | − | − |
| 91 | +++ | +++ | ++ |
| 92 | +++ | +++ | ++ |
| 93 | +++ | +++ | ++ |
| 94 | +++ | +++ | +++ |
| 95 | +++ | +++ | ++ |
| 96 | +++ | +++ | + |
| 97 | +++ | +++ | ++ |
| 98 | +++ | +++ | ++ |
| 99 | +++ | +++ | − |
| 100 | +++ | +++ | ++ |
| 101 | +++ | +++ | ++ |
| 102 | +++ | +++ | ++ |
| 103 | +++ | − | − |
| 104 | +++ | +++ | − |
| 105 | +++ | ++ | − |
| 106 | +++ | ++ | − |
| 107 | +++ | − | − |
| 108 | ++ | − | − |
| 109 | + | − | − |
| 110 | ++ | − | − |
| 111 | +++ | +++ | − |
| 112 | +++ | − | − |
| 113 | +++ | +++ | ++ |
| 114 | +++ | +++ | − |
| 115 | +++ | +++ | +++ |
| 116 | +++ | +++ | − |
| 117 | +++ | +++ | ++ |
| 118 | +++ | +++ | ++ |
| 119 | +++ | +++ | ++ |
| 120 | +++ | +++ | ++ |
| 121 | +++ | +++ | − |
| 122 | +++ | +++ | ++ |
| 123 | +++ | +++ | + |
| 124 | +++ | +++ | ++ |
| 125 | +++ | +++ | ++ |
| 126 | +++ | ++ | − |
| 127 | +++ | ++ | − |
| 128 | +++ | ++ | + |
| 129 | +++ | +++ | − |
| 130 | +++ | +++ | − |
| 131 | +++ | +++ | ++ |
| 132 | +++ | +++ | ++ |
| 133 | +++ | +++ | ++ |
| 134 | +++ | +++ | − |
| 135 | +++ | − | − |
| 136 | +++ | ++ | ++ |
| 137 | +++ | +++ | ++ |
| 138 | +++ | +++ | +++ |
| 139 | +++ | +++ | + |
| 140 | + | + | − |
| 141 | +++ | +++ | + |
| 142 | ++ | − | − |
| 143 | +++ | ++ | − |
| 144 | +++ | +++ | ++ |
| 145 | +++ | ++ | + |
| 146 | +++ | − | − |
| 147 | +++ | +++ | ++ |
| 148 | +++ | +++ | ++ |
| 149 | +++ | +++ | ++ |
| 150 | +++ | +++ | + |
| 151 | +++ | ++ | − |
| 152 | +++ | +++ | + |

| Note: | |
|---|---|
| pIC50 (p-P70S6K-(T389)) | |
| ≥9 | +++ |
| 9 > pIC50 ≥ 8 | ++ |
| 8 > pIC50 ≥ 6 | + |
| <6 | − |
| pIC50 (p-4E-NP1-(T37/46) or p-AKT1/2/3-(S473)) | |
| ≥8.5 | +++ |
| 8.5 > pIC50 ≥ 7.5 | ++ |
| 7.5 > pIC50 ≥ 6.0 | + |
| <6 | − |

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A method for delaying or preventing acquired resistance to a RAS inhibitor in a subject in need thereof, comprising administering to the subject an effective amount of a bi-steric inhibitor of mTOR, wherein the subject has already received or will receive administration of the RAS inhibitor, wherein the effective amount is an amount effective to delay or prevent acquired resistance to the RAS inhibitor in a subject in need thereof.

2. A method of treating acquired resistance to a RAS inhibitor in a subject in need thereof, comprising administering to the subject an effective amount of a bi-steric inhibitor of mTOR, wherein the effective amount is an amount effective to treat acquired resistance to the RAS inhibitor in a subject in need thereof.

3. The method of a claim 1 or claim 2, further comprising administering to the subject an effective amount of the RAS inhibitor.

4. The method of any one of claims 1-3, wherein the RAS inhibitor targets a specific RAS mutation.

5. The method of any one of claims 1-4, wherein the RAS inhibitor targets a KRAS mutation.

6. The method of any one of claims 1-5, wherein the RAS inhibitor targets the KRAS^{G12C} mutation.

7. The method of any one of claims 1-6, wherein the RAS inhibitor is a KRAS(OFF) inhibitor.

8. The method of claim 7, wherein the KRAS(OFF) inhibitor is selected from AMG 510, MRTX849, JDQ443 and MRTX1133, or a pharmaceutically acceptable salt thereof.

9. The method of any one of claims 1-8, wherein the bi-steric inhibitor of mTOR is RM-006, also known as RMC-6272, or RMC-5552, or a pharmaceutically acceptable salt thereof.

10. The method of any one of claims 1-9, wherein the bi-steric inhibitor of mTOR is a compound having the formula 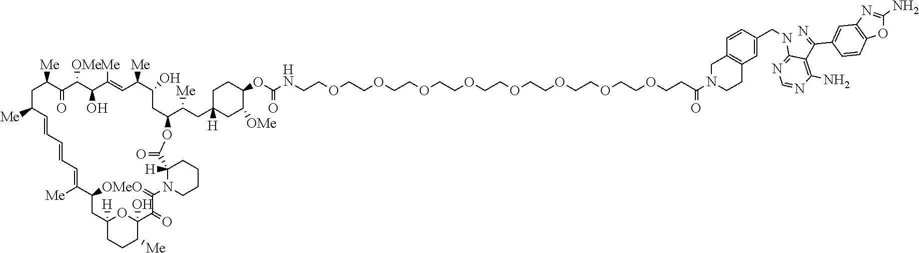 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]
or a stereoisomer or tautomer thereof.

11. The method of any one of claim 1-6 or 9-10, wherein the RAS inhibitor is a KRAS(ON) inhibitor.

12. The method of claim 11, wherein the KRAS(ON) inhibitor is a KRAS^{G12C}(ON) inhibitor.

13. The method of any one of claims 1-12, wherein the subject is administered the RAS inhibitor to treat or prevent a cancer.

14. The method of 13, wherein the cancer comprises a KRAS^{G12C} mutation.

15. The method of claim 13 or 14, wherein the cancer comprises co-occurring KRAS^{G12C} and STK11 mutations.

16. The method of any one of claims 13-15, wherein the cancer is a Non-Small Cell Lung Cancer (NSCLC) or a colorectal cancer.

17. The method of any one of claims 13-16, wherein the cancer comprises co-occurring KRAS^{G12C} and PIK3CA^{E545K} mutations.

18. The method of any one of claims 13-17, wherein the cancer is a colorectal cancer.

19. The method of any one of claims 1-18, wherein the method results in tumor regression.

20. The method of any one of claims 1-18, wherein the method results in tumor apoptosis.

21. A method of treating a subject having a cancer comprising administering to the subject an effective amount of a bi-steric inhibitor of mTOR in combination with a RAS inhibitor.

22. The method of claim 21, wherein the RAS inhibitor targets a specific RAS mutation.

23. The method of claim 21 or 22, wherein the RAS inhibitor targets a KRAS mutation.

24. The method of any one of claims 21-23, wherein the RAS inhibitor targets the KRAS^{G12C} mutation.

25. The method of any one of claims 21-24, wherein the RAS inhibitor is a KRAS(OFF) inhibitor.

26. The method of claim 25, wherein the KRAS(OFF) inhibitor is selected from AMG 510, MRTX849, JDQ443 and MRTX1133, or a pharmaceutically acceptable salt thereof.

27. The method of any one of claims 21-26, wherein the bi-steric inhibitor of mTOR is RM-006, also known as RMC-6272, or RMC-5552, or a pharmaceutically acceptable salt thereof.

28. The method of any one of claims 21-26, wherein the bi-steric inhibitor of mTOR is a compound having the formula 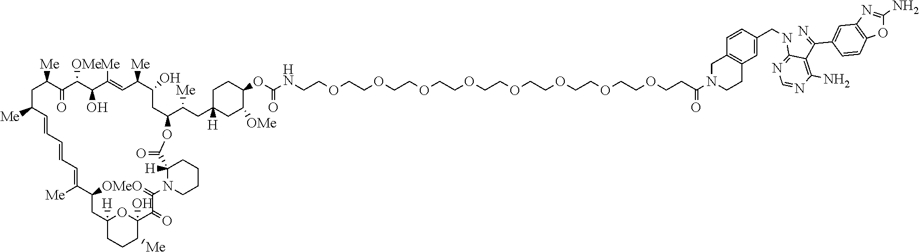 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]
or a stereoisomer or tautomer thereof.

29. The method of any one of claim 21-24, 27 or 28, wherein the RAS inhibitor is a KRAS(ON) inhibitor.

30. The method of claim 29, wherein the KRAS(ON) inhibitor is a KRAS^{G12C}(ON) inhibitor.

31. The method of any one of claims 21-30, wherein the cancer comprises a KRAS^{G12C} mutation.

32. The method of any one of claims 21-31, wherein the cancer comprises co-occurring KRAS^{G12C} and STK11 mutations.

33. The method of any one of claims 21-32, wherein the cancer is a Non-Small Cell Lung Cancer (NSCLC).

34. The method of any one of claims 21-33, wherein the cancer comprises co-occurring KRAS^{G12C} and PIK3CA^{E545K} mutations.

35. The method of any one of claim 21-32 or 34, wherein the cancer is a colorectal cancer.

36. The method of any one of claims 21-35, wherein the method results in tumor regression.

37. The method of any one of claims 21-36, wherein the method results in tumor apoptosis.

38. A method of inducing apoptosis of a tumor cell comprising contacting the tumor cell with an effective amount of a bi-steric inhibitor of mTOR in combination with a RAS inhibitor, wherein the effective amount is an amount effective to induce apoptosis of the tumor cell.

39. The method of any one of claims 1-38, wherein the method results in an improved lifespan for the subject as compared to the lifespan of a similar subject that has not received a treatment with the RAS inhibitor and the bi-steric mTOR inhibitor.
